(19) United States
(12) Patent Application Publication
(10) Pub. No.: US 2025/0163048 A1
(43) Pub. Date: May 22, 2025

(54) COMPOUNDS AND METHODS OF USE

(71) Applicant: Tango Therapeutics, Inc., Boston, MA (US)

(72) Inventor: Kevin M. COTTRELL, Boston, MA (US)

(21) Appl. No.: 18/833,319

(22) PCT Filed: Jan. 26, 2023
(86) PCT No.: PCT/US2023/011659

§ 371 Date: Jul. 25, 2024

Related U.S. Application Data
(60) Provisional application No. 63/303,368, filed on Jan. 26, 2022.
(60) Provisional application No. 63/435,212, filed on Dec. 23, 2022.

(51) Int. Cl. C07D 471/04; A61K 31/4545; A61K 31/4709; A61K 31/4725; A61K 31/496; A61K 31/5377; A61K 45/06; C07D 491/048; C07D 519/00

(52) U.S. Cl. CPC C07D 471/04; A61K 31/4545; A61K 31/4709; A61K 31/4725; A61K 31/496; A61K 31/5377; A61K 45/06; C07D 491/048; C07D 519/00

(57) ABSTRACT

Provided are compounds of Formula (A) and Formula (I): (I), and pharmaceutically acceptable salts thereof, and pharmaceutical compositions, processes of preparing and methods of treating thereof; wherein Ring A, R^{B}, Ring B, R¹, R² and n are as defined in any of the embodiments described herein.

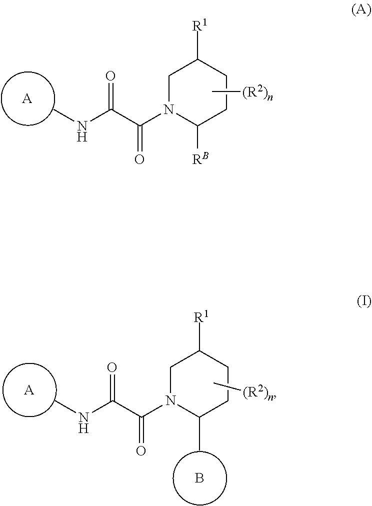

### CROSS-REFERENCE TO RELATED APPLICATIONS

This application claims priority to U.S. Provisional Application No. 63/303,368, filed on Jan. 26, 2022, and U.S. Provisional Application No. 63/435,212, filed on Dec. 23, 2022, which are incorporated by reference herein in their entireties and for all purposes.

### FIELD

Provided herein are compounds, and compositions and methods thereof. In some embodiments, provided are compounds for inhibiting protein arginine methyltransferase 5 (PRMT5). In some embodiments, provided are methods for treatment of diseases or disorders, such as cancer.

### BACKGROUND

Protein arginine methyltransferase 5 (PRMT5) is a type II arginine methyltransferase that regulates essential cellular functions, including the regulation of cell cycle progression, apoptosis and the DNA-damage response (Koh, C. et al., Curr Mol Bio Rep 2015; Wu et al., Nat Rev Drug Discovery 2021). MTAP is a critical enzyme in the methionine salvage pathway, a six-step process that recycles methionine from the product of polyamine synthesis, methylthioadenosine (MTA). Loss of MTAP causes the accumulation of its substrate, MTA, which has been described to function as a SAM-competitive PRMT5 inhibitor (Kruykov et al., 2016; Marjon et al., 2016 and Markarov et al., 2016). Data from genome-wide genetic perturbation screens using shRNA suggests a selective requirement for PRMT5 activity particularly in MTAP-deleted cancer cell lines (Kruykov et al., 2016; Marjon et al., 2016 and Markarov et al., 2016). It is proposed that the accumulation of MTA caused by MTAP-deletion in these cell lines partially inhibits PRMT5, rendering those cells selectively sensitive to additional PRMT5 inhibition.

A PRMT5 inhibitor that leverages the accumulation of MTA by binding in an MTA-uncompetitive, non-competitive or mixed mode manner or in a MTA-cooperative binding manner may demonstrate selectivity for MTAP-deleted tumor cells. Some PRMT5 inhibitors are currently being explored for therapeutic uses (e.g., for treating cancer), however there are currently no such PRMT5 therapies approved by the United States Food and Drug Administration that demonstrate selectivity for MTAP-deleted cancer cell lines.

Accordingly, there is a need for PRMT5 inhibitors for treating diseases, such as cancers.

### SUMMARY

In one embodiment, provided is a compound of Formula (I) or Formula (A):
or a pharmaceutically acceptable salt thereof,
   wherein:

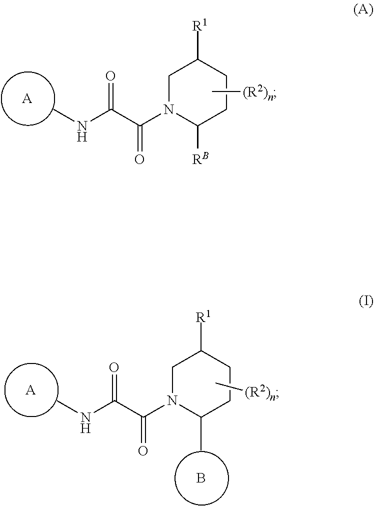

Ring A is an optionally substituted fused bicyclic 8-10 membered heteroaryl ring system containing at least one nitrogen atom, wherein the 8-10 membered refers to the total number of atoms in the fused system;
R^{B} is an optionally substituted C₁-C₆ alkyl;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
   each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
   each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of —O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
   n is 0, 1, 2 or 3;
provided that:
(i) when the compound is of Formula (A), Ring A is a 3-pyridine fused with a 5-membered heteroaryl via 2 carbon atoms; and
(ii) the compound is not one of compounds a. to d. or a pharmaceutically acceptable salt thereof:
   a. N-(8-fluoroquinolin-3-yl)-2-oxo-2-(2-phenylpiperidin-1-yl)acetamide

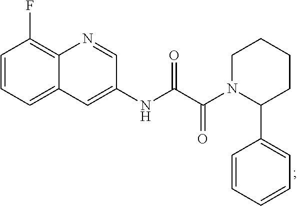 [SMILES: O=C(Nc1cnc2c(F)cccc2c1)C(=O)N1CCCCC1c1ccccc1]

b. 2-(2-(1H-pyrazol-4-yl)piperidin-1-yl)-N-(1H-indol-4-yl)-2-oxoacetamide

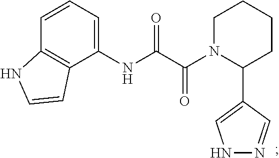 [SMILES: O=C(Nc1cccc2[nH]ccc12)C(=O)N1CCCCC1c1cn[nH]c1]

c. 2-(2-(4-bromophenyl)piperidin-1-yl)-2-oxo-N-(pyrazolo[1,5-a]pyrimidin-3-yl)acetamide

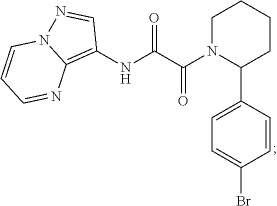 [SMILES: O=C(Nc1cnn2cccnc12)C(=O)N1CCCCC1c1ccc(Br)cc1]

d. 2-(2-(1H-pyrazol-4-yl)piperidin-1-yl)-N-(1H-indazol-5-yl)-2-oxoacetamide

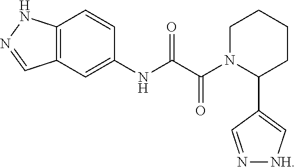 [SMILES: O=C(Nc1ccc2[nH]ncc2c1)C(=O)N1CCCCC1c1cn[nH]c1]

In one embodiment, provided is a pharmaceutical composition comprising a compound of Formula (A) or Formula (I) to (III7a), or a pharmaceutically acceptable salt thereof, as defined in any of the embodiments described herein and a pharmaceutically acceptable carrier. In some embodiments, the pharmaceutical composition further comprises a second therapeutic agent.

In one embodiment, provided is a method of treating an MTAP-deficient and/or an MTA-accumulating disease in a subject in need thereof by administering to the subject an effective amount (e.g., a therapeutically effective amount) of compound of Formula (A) and Formula (I) to (III7a), or a pharmaceutically acceptable salt thereof, as defined in any of the embodiments described herein or a pharmaceutically acceptable composition thereof. In some embodiments, the compound or composition is administered in combination with a second therapeutic agent.

In one embodiment, provided is a method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a compound of Formula (A) or Formula (I) to (III7a) as defined herein or a pharmaceutical composition thereof to the subject identified in step b).

In an embodiment, provided is a use of a compound of Formula (A) or Formula (I) to (III7a), or a pharmaceutically acceptable salt thereof, as defined in any of the embodiments described herein, or of a pharmaceutically acceptable composition as described herein for treating an MTAP-deficient and/or an MTA-accumulating disease in a subject in need thereof. In an embodiment, the compound or composition is configured to be administered in combination with a second therapeutic agent.

In an embodiment, provided is a compound of Formula (A) or Formula (I) to (III7a), or a pharmaceutically acceptable salt thereof, as defined in any of the embodiments described herein, or a pharmaceutically acceptable composition as described herein for treating an MTAP-deficient and/or an MTA-accumulating disease in a subject in need thereof. In an embodiment, the compound or composition is configured to be administered in combination with a second therapeutic agent.

In an embodiment, provided is a use of a compound of compound of Formula (A) or Formula (I) to (III7a), or a pharmaceutically acceptable salt thereof, as defined in any of the embodiments described herein, or of a pharmaceutically acceptable composition as described herein in the manufacturing of a medicament for treating an MTAP-deficient and/or an MTA-accumulating disease in a subject in need thereof. In an embodiment, the medicament is configured to be administered in combination with a second therapeutic agent.

### DETAILED DESCRIPTION

The disclosure herein sets forth exemplary methods, parameters and the like. It should be recognized, however, that such description is not intended as a limitation on the scope of the present disclosure but is instead provided as a description of exemplary embodiments.

As generally described herein, provided are compounds (e.g., compounds of Formula (A) and Formula (I) to (III7a) or compounds of Table 1, or pharmaceutically acceptable salts thereof) that are MTA-uncompetitive PRMT5 inhibitors useful for treating proliferating disorders (e.g., cancers) associated with MTAP deficiencies and/or MTA accumulation.

In some embodiments, provided are compounds (e.g., compounds of Formula (A) and Formula (I) to (III7a) or compounds of Table 1, or pharmaceutically acceptable salts thereof) that are MTA-uncompetitive, non-competitive or mixed mode PRMT5 inhibitor or an MTA cooperative binding agent useful for treating proliferating disorders (e.g., cancers) associated with MTAP deficiencies and/or MTA accumulation.

### Definitions

As used in the present disclosure, the following words and phrases are generally intended to have the meanings as set forth below unless expressly indicated otherwise or the context in which they are used indicates otherwise.

### MTAP

“MTAP” as used herein refers to methylthioadenosine phosphorylase, an enzyme in the methionine salvage pathway, also known as S-methyl-5′-thioadenosine phosphorylase; also known as BDMF; DMSFH; DMSMFH; LGMBF; MSAP; and c86fus. External IDs: OMIM: 156540 MGI: 1914152 HomoloGene: 1838 chEMBL: 4941 GeneCards: MTAP Gene; Entrez 4507; RefSeq (mRNA): NM_002451; location: Chr 9: 21.8-21.93 Mb. By “wild-type” MTAP is meant that encoded by NM_002451 or having the same amino acid sequence (NP_002442). (Schmid et al. Oncogene 2000, 19, pp 5747-54).

As used herein, the term “MTAP-deficient”, “MTAP-deficiency”, “MTAP-null” and the like refer to cells (including, but not limited to, cancer cells, cell lines, tissues, tissue types, tumors, etc.) that have a significant reduction in post-translational modification, production, expression, level, stability and/or activity of MTAP relative to that in a control, e.g., reference or normal or non-cancerous cells. The reduction can be at least about 20%, 30%, 40%, 50%, 60%, 70%, 80% or 90%. In some embodiments, the reduction is at least 20%. In some embodiments, the reduction is at least 50%. The terms “MTAP-deficient and/or MTA accumulating”, “MTAP-deficient and/or MTA-accumulating”, MTAP deficient and/or MTA upregulated” and the like, regarding a cell or cells, etc., indicate that the cell or cells, etc., either are deficient in MTAP and/or overproduce or accumulate MTA. MTAP-deficient cells include those wherein the MTAP gene has been mutated, deleted, or transcriptionally silenced. As a non-limiting example, MTAP-deficient cells can have a homozygous deletion. MTAP knockdown is not lethal. In some embodiments, the MTAP-deficient cells are also CDKN2A-deficient. The MTAP deficiency can be detected using any reagent or technique known in the art, for example: immunohistochemistry utilizing an antibody to MTAP, and/or genomic sequencing, and/or nucleic acid hybridization and/or amplification utilizing at least one probe or primer comprising a sequence of at least 12 contiguous nucleotides (nt) of the sequence of MTAP, wherein the primer is no longer than about 30 nt.

An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not. Similarly, some disease cells may be MTA-accumulating while others are not. Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor. Some cancer cells which are MTAP-deficient are also deficient in CDKN2A; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

### MTA and MTA Accumulation

By “MTA” is meant the PRMT5 inhibitor also known as methyl-thioadenosine, S-methyl-5′-thioadenosine, [5′deoxy-5′-(methylthio)-fl-D-ribofuranosyl] adenine, 5′-methyl-thioadenosine, 5′-deoxy, 5′-methyl thioadenosine, and the like. MTA selectively inhibits PRMT5 methyltransferase activity. MTA is the sole known catabolic substrate for MTAP. The terms “MTA accumulating”, “MTA overproducing”, “MTA upregulated” and the like refer to cells (including, but not limited to, cancer cells, cell lines, tissues, tissue types, tumors, etc.) that have a significantly increased production, level and/or stability of MTA. MTA-accumulating cells include those wherein the cells comprise at least about 20%, 30%, 40%, 50%, 60%, 70%, 80%, 90%, 100%, or greater than 100%, higher production, level and/or stability of MTA than that in normal or non-cancerous cells. In some embodiments, MTA-accumulating cells include those wherein the cells comprise at least 20% higher production, level and/or stability of MTA than that in normal or non-cancerous cells. In some embodiments, MTA-accumulating cells include those wherein the cells comprise at least 50% higher production, level and/or stability of MTA than that in normal or non-cancerous cells. Determination of MTA accumulation in test samples (e.g., cells such as cancer cells being tested for MTA accumulation) and reference samples, and other cells, tissues, samples, etc., can be performed using any method known in the art. Such methods for detecting MTA include, as a non-limiting example, liquid chromatography-electrospray ionization-tandem mass spectrometry (LC-ESI-MS/MS), as described in Stevens et al. J. Chromatogr. A. 2010, 1217, pp 3282-3288; and Kirovski et al. Am. J. Pathol. 2011, 178, pp 1145-1152; and references cited therein. Loss of MTAP is associated with accumulation of MTA (Williams-Ashman et al. Biochem. Pharm. 1982, 31, pp 277-288; and Limm et al. Eur. J. Cancer. 2013, 49, Issue 6.

An “MTA-accumulation-related”, “MTA-accumulation”, “MTA-accumulating”, “MTA overproducing”, “MTA upregulated” disease (for example, a proliferating disease, e.g., a cancer) or a disease (for example, a proliferating disease, e.g., a cancer) “associated with MTA accumulation” or a disease (for example, a proliferating disease, e.g., a cancer) “characterized by MTA accumulation” and the like refer to an ailment (for example, a proliferating disease, e.g., a cancer) wherein a significant number of cells are MTA accumulating. Examples of MTA-accumulating diseases include, but are not limited to, cancers, including but not limited to: glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not.

In a patient having or having been diagnosed with an MTA-accumulating disease, some cells may be MTA-accumulating while others are not.

An increase in therapeutic window between normal cells and MTAP-deleted/MTA accumulating cells could be achieved by using an inhibitor that binds PRMT5 uncompetitively with MTA. As used herein, “uncompetitive binding” and “uncompetitive inhibition” and “cooperative binding” and “cooperative inhibition” (e.g., MTA-uncompetitive binding, MTA-uncompetitive inhibition, MTA-cooperative binding, MTA-cooperative inhibition) refers to binding of an inhibitor to a protein (e.g., PRMT5) that is increased in the presence of a co-factor (e.g., MTA) over the binding of the same inhibitor in the absence of the co-factor. The PRMT5 inhibitors known in the art are generally either SAM (S-adenosylmethionine) uncompetitive or SAM competitive. As the concentration of SAM in wild-type and MTAP-null cells is similar, these inhibitors are expected to bind with similar potency to both cell types. By contrast, an MTA-cooperative (and either SAM competitive or showing enhanced cooperativity with MTA relative to SAM) inhibitor would bind with apparent greater potency in the presence of high concentrations of MTA and would therefore result in preferential inhibition of PRMT5 in MTA-accumulating cells relative to normal cells.

As described further herein, a cancer cell, a cancer type, or a subject with cancer, is “PRMT5 inhibitor sensitive,” sensitive to treatment with PRMT5 inhibitors,” sensitive to PRMT5 therapeutic inhibition,” or described in similar terms if it is amenable to treatment with a PRMT5 inhibitor, e.g., due to its MTAP deficiency and/or MTA accumulation character.

### PRMT5

“PRMT5” as used herein is the gene or protein Protein Arginine Methyltransferase 5, also known as HRMTIL5; IBP72; JBP1; SKB1; or SKB1Hs External IDs: OMIM: 604045, MGI: 1351645, HomoloGene: 4454, ChEMBL: 1795116, GeneCards: PRMT5 Gene; EC number 2.1.1.125. Ensembl ENSG00000100462; UniProt 014744; Entrez Gene ID: 10419; RefSeq (mRNA): NM_001039619. The mouse homolog is NM_013768. Methyltransferases such as PRMT5 catalyze the transfer of one to three methyl groups from the co-factor S-adenosylmethionine (also known as SAM or AdoMet) to lysine or arginine residues of histone proteins. Arginine methylation is carried out by 9 different protein arginine methyltransferases (PRMT) in humans. Three types of methylarginine species exist: (1) Monomethylarginine (MMA); (2) Asymmetric dimethyl arginine (ADMA), which is produced by Type I methyl transferases (PRMT1, PRMT2, PRMT3, CARMI, PRMT6 and PRMT8); and (3) Symmetrical dimethylarginine (SDMA), which is produced by Type II methyl transferases (PRMT5 and PRMT7). PRMT1 and PRMT5 are the major asymmetric and symmetric arginine methyltransferases, respectively. PRMT5 promotes symmetric dimethylation on histones at H3R8 and H4R3 (H4R3me2). Symmetric methylation of H4R3 is associated with transcriptional repression and can act as a binding site for DNMT3A. Loss of PRMT5 results in reduced DNMT3A binding and gene activation. Tumor suppressor gene ST7 and chemokines RNATES, IP10, CXCL11 are targeted and silenced by PRMT5. WO 2011/079236.

Additional substrates include E2F1, p53, EGFR and CRAF. PRMT5 is part of a multi-protein complex comprising the co-regulatory factor WDR77 (also known as MEP50, a CDK4 substrate) during G1/S transition. Phosphorylation increases PRMT5/WDR77 activity. WDR77 is the non-catalytic component of the complex and mediates interactions with binding partners and substrates. PRMT5 can also interact with pICIn or RioK1 adaptor proteins in a mutually exclusive fashion to modulate complex composition and substrate specificity.

PRMT5 has either a positive or negative effect on its substrates by arginine methylation when interacting with a number of complexes and is involved in a variety of cellular processes, including RNA processing, signal transduction, transcriptional regulation, and germ cell development. PRMT5 is a major pro-survival factor regulating eIF4E expression and p53 translation. PRMT5 triggers p53-dependent apoptosis and sensitized various cancer cells to Tumor necrosis factor (TNF)-related apoptosis-inducing ligand (TRAIL) without affecting TRAIL resistance in non-transformed cells.

The term “PRMT5 inhibitor” refers to any compound capable of inhibiting the production, level, activity, expression or presence of PRMT5. These include, as non-limiting examples, any compound inhibiting the transcription of the gene, the maturation of RNA, the translation of mRNA, the posttranslational modification of the protein, the enzymatic activity of the protein, the interaction of same with a substrate, etc. The term also refers to any agent that inhibits the cellular function of the PRMT5 protein, either by ATP-competitive inhibition of the active site, allosteric modulation of the protein structure, disruption of protein-protein interactions, or by inhibiting the transcription, translation, post-translational modification, or stability of PRMT5 protein.

In some embodiments, a PRMT5 inhibitor competes with another compound, protein or other molecule which interacts with PRMT5 and is necessary for PRMT5 function. As a non-limiting example, a PRMT5 inhibitor can compete with the co-factor S-adenosylmethionine (also known as SAM or AdoMet).

In some embodiments, the PRMT5 inhibitor is uncompetitive with MTA. In some embodiments, the PRMT5 inhibitor is uncompetitive with MTA and competitive with SAM. In some embodiments, the PRMT5 inhibitor is uncompetitive with MTA and uncompetitive with SAM but binds with a higher degree of potency for the MTA complex relative to the SAM complex.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

$$e . e . = ( 90 - 10 ) / 100 = 80 \% .$$

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

$$d . e . = ( 90 - 10 ) / 100 = 80 \% .$$

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃.

“Aliphatic” refers to an alkyl, alkenyl, alkynyl, or carbocyclyl group, as defined herein. “Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group (e.g., a 3-10 membered heterocyclyl containing 1, 2 or 3 heteroatoms selected from N, O, S and oxidized forms thereof). In some embodiments, a heterocyclylalkyl is a C₁₋₂ alkyl-heterocyclyl (e.g., —CH₂-heterocyclyl, —CH₂CH₂-heterocyclyl, —CH(CH₃)-heterocyclyl). In some embodiments, a heterocyclylalkyl is a —CH₂-heterocyclyl. Typical heterocyclylalkyl groups include, but are not limited to, tetrahydrofuranylmethyl, tetrahydropyranylmethyl, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Aralkyl” or “arylalkyl” is a subset of alkyl and aryl, as defined herein, and refers to an alkyl group substituted by an aryl group (e.g., a C₆-C₁₀ aryl group). In some embodiments, arylalkyl is a C₁₋₂ alkyl-aryl (e.g., —CH₂-aryl, —CH₂CH₂-aryl, —CH(CH₃)-aryl). In some embodiments, arylalkyl is a —CH₂-aryl (e.g., —CH₂-phenyl, —CH₂-naphthyl).

“Alkyl” refers to a radical of a straight-chain or branched saturated hydrocarbon group having from 1 to 20 carbon atoms (“C₁₋₂₀ alkyl” or “C₁-C₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl. Common alkyl abbreviations include Me (—CH₃), Et (—CH₂CH₃), ⁱPr (—CH(CH₃)₂), ⁿPr (—CH₂CH₂CH₃), ⁿBu (—CH₂CH₂CH₂CH₃), or ⁱBu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

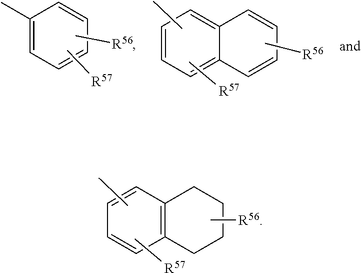

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, —NR⁵⁸COR⁵⁹, —NR⁵⁸SOR⁵⁹NR⁵⁸SO₂R⁵⁹, —COOalkyl, —COOaryl, —CONR⁵⁸R⁵⁹, —CONR⁵⁸OR⁵⁹, —NR⁵⁸R⁵⁹, —SO₂NR⁵⁸R⁵⁹, —S-alkyl, —SOalkyl, —SO₂alkyl, —Saryl, —SOaryl, —SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, —C₁-C₈ alkyl, —C₁-C₄ haloalkyl, —C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring. In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

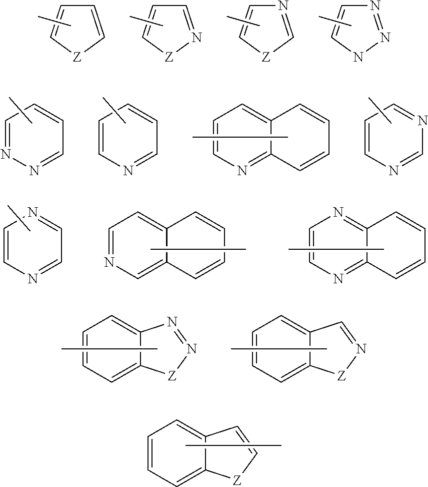

wherein each Z is selected from carbonyl, N, NR⁶⁵, O, and S; and R⁶⁵ is independently hydrogen, —C₁-C₈ alkyl, C₃-C₁₀ cycloalkyl, 4-10 membered heterocyclyl, C₆-C₁₀ aryl, and 5-10 membered heteroaryl.

In the structures described herein, a substituent attached to a polycyclic (e.g., bicyclic or tricyclic)cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group (e.g., a 5-10 membered heteroaryl containing 1, 2 or 3 heteroatoms selected from O, N and S and oxidized forms thereof), wherein the point of attachment is on the alkyl moiety. In some embodiments, a heteroarylalkyl is a C₁₋₂ alkyl-heteroaryl (e.g., —CH₂-heteroaryl, —CH₂CH₂-heteroaryl, —CH(CH₃)-heteroaryl). In some embodiments, a heteroarylalkyl is a —CH₂-heteroaryl. Typical heteroarylalkyl groups include, but are not limited to, pyridinylmethyl, pyrimidinylmethyl, furanylmethyl, thiophenylmethyl, pyrrolylmethyl, pyrazolylmethyl, imidazolylmethyl, thiazolylmethyl, oxazolylmethyl, thiazolylmethyl, pyridinylethyl, pyrimidinylethyl, furanylethyl, thiophenylethyl, pyrrolylethyl, pyrazolylethyl, imidazolylethyl, thiazolylethyl, oxazolylethyl, thiazolylethyl and the like.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄ tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₈₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₅), spiro[2.2]pentane (C₅), bicyclo[2.1.0]pentane (C), bicyclo[2.1.1]hexane (C₆), bicyclo[3.1.0]hexane (C₆), spiro[2.3]hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4]heptane (C₇), spiro[3.3]heptane (C₇), bicyclo[2.2.2]octane (C₈), bicyclo[4.1.1]octane (C₈)octahydropentalene (C₈), bicyclo[3.2.1]octane (C₅), bicyclo[4.2.0]octane (C₅), spiro[2.5]octane (C₈), spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂).

In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C₁₀₋₁₂ tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In some embodiments, the heterocyclyl is a 3- to 10-membered non-aromatic ring system having ring carbon atoms and 1, 2 or 3 heteroatoms independently selected from nitrogen, oxygen and sulfur, including oxidized forms thereof. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. “Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂): (C₆-C₁₀ aryl), —C(═O)—(CH₂)ₜ (5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and —N(alkyl)₂ radicals respectively. In some embodiments the alkylamino is a —NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, —C₁-C₈ alkyl, —C₃-C₈ alkenyl, —C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ (5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{cc}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 Rad groups;
each instance of R^{bb} is, independently, selected from hydrogen, —OH, —OR^{aa}, —N(R)₂, —CN, —C(═O)R^{aa}, —C(═O)N(R^{cc})₂, —CO₂R^{aa}, —SO₂R^{aa}, —C(═NR^{cc})OR^{aa}, C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion.
each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂ (C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R&8 substituents can be joined to form ═O or ═S; wherein X⁻ is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl) propanamide, 3-(o-nitrophenyl) propanamide, 2-methyl-2-(o-nitrophenoxy) propanamide, 2-methyl-2-(o-phenylazophenoxy) propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo) fluorenylmethyl carbamate, 9-(2,7-dibromo) fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido) propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl) mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten) acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein.

Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl) methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl) xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy) butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(RC)₃⁺X⁻, —P(OR)₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, —Cl, —Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, —OTs), methanesulfonate (mesylate, —OMs), p-bromobenzenesulfonyloxy (brosylate, —OBs), —OS(═O)₂(CF₂)₃CF₃ (nonaflate, —ONf), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O) NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NR^{bb})R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}s (═O)₂Ra, ═NR^{bb}, or ═NOR^{cc}; each instance of R^{aa} is, independently, selected from C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{cc}, —C(═O)N(R^{cc})₂, —CO₂R^{aa}, —SO₂R^{cc}, —C(═NR^{cc})OR^{aa}, —C(═NR^{cc})N(R^{cc})₂, —SO₂N(RC)₂, —SO₂R^{cc}, —SO₂OR^{cc}, —SOR^{aa}, —C(═S)N(R^{cc})₂, —C(═O)SR, —C(═S)SR^{cc}, —P(═O)₂R^{aa}, —P(═O)(R^{aa})₂, —P(═O)₂N(R^{cc})₂, —P(═O)(NR^{cc})₂, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, —C₁₋₆ alkyl, —C₁₋₆ haloalkyl, —C₂₋₆ alkenyl, —C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, —C₁₋₆ haloalkyl, —C₂₋₆ alkenyl, —C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, —C₁₋₆ alkyl, —C₁₋₆ haloalkyl, —C₂₋₆ alkenyl, —C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂ (C₁₋₆ alkyl) “X”, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂ (C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)₂ (C₁₋₆ alkyl), —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ haloalkyl, —C₂₋₆ alkenyl, —C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(RC)₂, —SO₂R^{cc}, —SO₂OR^{cc}, —SOR^{aa}, —C(═S)N(RC)₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, —C₁₋₁₀ alkyl, —C₁₋₁₀ haloalkyl, —C₂₋₁₀ alkenyl, —C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above. These and other exemplary substituents are described in more detail in the Detailed Description, Examples, and Claims. The invention is not intended to be limited in any manner by the above exemplary listing of substituents.

### Other Definitions

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgous monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compounds

Provided herein are compounds of Formula (I) and Formula (A). Unless the context requires otherwise, reference throughout this specification to “a compound of Formula (I) or Formula (A)” or “compounds of Formula (I) or Formula (A)” refers to all embodiments of Formula (I) and Formula (A), including, for example, compounds of Formula (A), Formula (I), Formula (Ia), Formula (Ib), Formula (Ic), Formula (Id), Formula (III), Formula (II1a), Formula (II2), Formula (II2a), Formula (II2b), Formula (II2c), Formula (II2d), Formula (II2e), Formula (II3), Formula (II3a), Formula (II3b), Formula (II3c), Formula (II3d), Formula (II3e), Formula (II3f), Formula (II3g), Formula (II3h), Formula (II4), Formula (II4a), Formula (II4b), Formula (II4c), Formula (II5), Formula (II5a), Formula (II6), Formula (II6a), Formula (II7), Formula (II7a), Formula (II7b), Formula (II7c), Formula (II7d), Formula (II8), Formula (II8a), Formula (II9), Formula (II9a), Formula (II10), Formula (II10a), Formula (II10b), Formula (II10c), Formula (II11), Formula (III1a), Formula (II11b), Formula (II11c), Formula (II11d), Formula (II11e), Formula (II12), Formula (II12a), Formula (II12b), Formula (II12c), Formula (III1), Formula (III1a), Formula (III1b), Formula (II11c), Formula (II11d), Formula (III2), Formula (III2a), Formula (III2b), Formula (III3), Formula (III3a), Formula (III3b), Formula (III4), Formula (III4a), Formula (III4b), Formula (III4c), Formula (III5), Formula (III5a), Formula (III5b), Formula (III6), Formula (III6a), Formula (III7), Formula (III7a), (i.e., Formula (I)-Formula (III7a)) as well as the compounds of Table 1.

In one embodiment, provided herein are compounds of Formula (I) and Formula (A) or pharmaceutically acceptable salts thereof,
wherein:

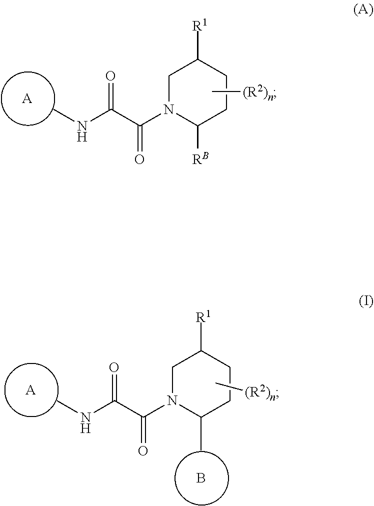

Ring A is an optionally substituted fused bicyclic 8-10 membered heteroaryl ring system containing at least one nitrogen atom, wherein the 8-10 membered refers to the total number of atoms in the fused system;
R^{B} is an optionally substituted C₁-C₆ alkyl;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
   provided that:
(i) when the compound is of Formula (A), Ring A is a 3-pyridine fused with a 5-membered heteroaryl via 2 carbon atoms; and
(ii) the compound is not one of compounds a. to d. or a pharmaceutically acceptable salt thereof:
   a. N-(8-fluoroquinolin-3-yl)-2-oxo-2-(2-phenylpiperidin-1-yl)acetamide

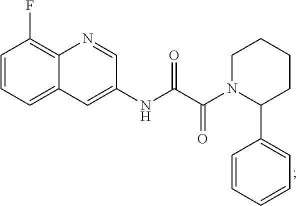 [SMILES: O=C(Nc1cnc2c(F)cccc2c1)C(=O)N1CCCCC1c1ccccc1]

b. 2-(2-(1H-pyrazol-4-yl)piperidin-1-yl)-N-(1H-indol-4-yl)-2-oxoacetamide

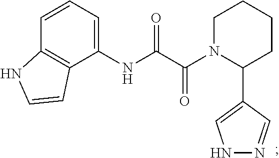 [SMILES: O=C(Nc1cccc2[nH]ccc12)C(=O)N1CCCCC1c1cn[nH]c1]

c. 2-(2-(4-bromophenyl)piperidin-1-yl)-2-oxo-N-(pyrazolo[1,5-a]pyrimidin-3-yl)acetamide

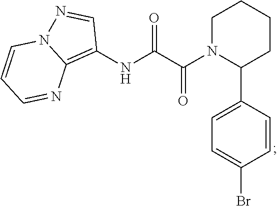 [SMILES: O=C(Nc1cnn2cccnc12)C(=O)N1CCCCC1c1ccc(Br)cc1]

d. 2-(2-(1H-pyrazol-4-yl)piperidin-1-yl)-N-(1H-indazol-5-yl)-2-oxoacetamide

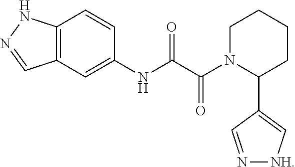 [SMILES: O=C(Nc1ccc2[nH]ncc2c1)C(=O)N1CCCCC1c1cn[nH]c1]

In one embodiment, provided herein are compounds of Formula (I) and Formula (A) or pharmaceutically acceptable salts thereof,
wherein:

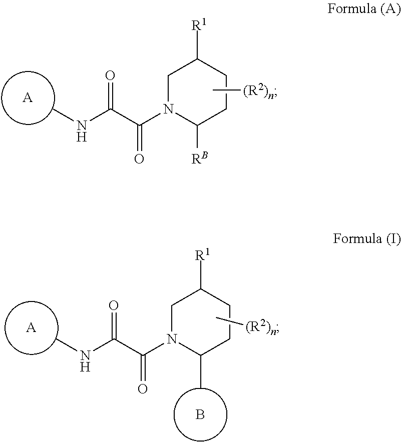

Ring A is a fused bicyclic 8-10 membered heteroaryl ring system containing at least one nitrogen atom, wherein the 8-10 membered refers to the total number of atoms in the fused system, substituted at any available positions with 0, 1, 2 or 3 instances of R⁴;
R^{B} is C₁-C₆ alkyl substituted with 0, 1, 2 or 3 substituents independently selected from ═O, —F, —Cl, —OH—, —OMe, —NH₂ and —CN;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is substituted at any available position with 0, 1, 2 or 3 instances of R³;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl);
each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein each alkyl, cycloalkyl, heteroalkyl, heterocyclyl, aryl, heteroaryl, cycloalkylalkyl, heterocyclylalkyl, arylalkyl and heteroarylalkyl of R³ is optionally substituted at any available position (e.g., substituted with 0, 1, 2, 3, 4, or 5 instances of R⁶, wherein each R⁶ is independently selected from the group consisting of —C₁-C₆ alkyl, —C₃-C₉ cycloalkyl, 3-10 membered heterocyclyl, —OH, ═O, halo, —OC₁-C₆ alkyl, —C₁-C₆ haloalkyl, —C(═O)C₁-C₆ alkyl, —N(C₁-C₆ alkyl)₂, —C₁-C₆ heteroalkyl, and —NHC(═O)C₁-C₆ alkyl, wherein one or more hydrogens of the —C₁-C₆ alkyl can be replaced with deuterium); and
each R^{a3} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl).
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂; and
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
   provided that:
(i) when the compound is of Formula (A), Ring A is a 3-pyridine fused with a 5-membered heteroaryl via 2 carbon atoms; and
(ii) the compound is not one of compounds a. to d. or a pharmaceutically acceptable salt thereof:
   a. N-(8-fluoroquinolin-3-yl)-2-oxo-2-(2-phenylpiperidin-1-yl)acetamide

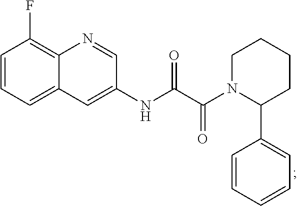 [SMILES: O=C(Nc1cnc2c(F)cccc2c1)C(=O)N1CCCCC1c1ccccc1]

b. 2-(2-(1H-pyrazol-4-yl)piperidin-1-yl)-N-(1H-indol-4-yl)-2-oxoacetamide

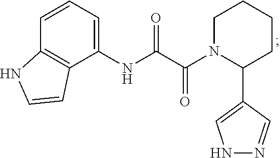 [SMILES: O=C(Nc1cccc2[nH]ccc12)C(=O)N1CCCCC1c1cn[nH]c1]

c. 2-(2-(4-bromophenyl)piperidin-1-yl)-2-oxo-N-(pyrazolo[1,5-a]pyrimidin-3-yl)acetamide

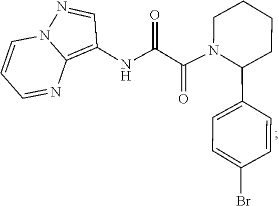 [SMILES: O=C(Nc1cnn2cccnc12)C(=O)N1CCCCC1c1ccc(Br)cc1]

d. 2-(2-(1H-pyrazol-4-yl)piperidin-1-yl)-N-(1H-indazol-5-yl)-2-oxoacetamide

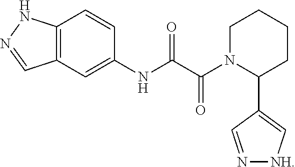 [SMILES: O=C(Nc1ccc2[nH]ncc2c1)C(=O)N1CCCCC1c1cn[nH]c1]

In some embodiments, the compound is of Formula (A). In some embodiments, the compound is of Formula (I).

In some embodiments, provided herein are compounds of Formula (I):

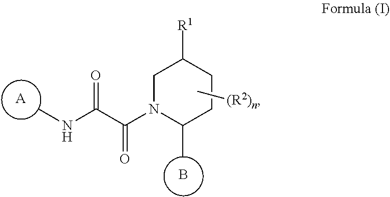

or a pharmaceutically acceptable salt thereof,
wherein:
   Ring A is an optionally substituted fused bicyclic 8-10 membered heteroaryl ring system containing at least one nitrogen atom, wherein the 8-10 membered refers to the total number of atoms in the fused system;
   Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
   each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
   each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
   each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
   n is 0, 1, 2 or 3;
provided that the compound is not one of compounds a. to d. or a pharmaceutically acceptable salt thereof:
   a. N-(8-fluoroquinolin-3-yl)-2-oxo-2-(2-phenylpiperidin-1-yl)acetamide

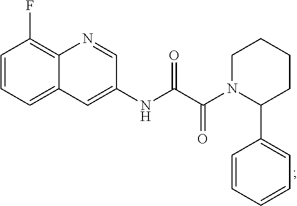 [SMILES: O=C(Nc1cnc2c(F)cccc2c1)C(=O)N1CCCCC1c1ccccc1]

b. 2-(2-(1H-pyrazol-4-yl)piperidin-1-yl)-N-(1H-indol-4-yl)-2-oxoacetamide

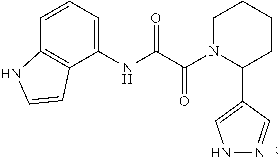 [SMILES: O=C(Nc1cccc2[nH]ccc12)C(=O)N1CCCCC1c1cn[nH]c1]

c. 2-(2-(4-bromophenyl)piperidin-1-yl)-2-oxo-N-(pyrazolo[1,5-a]pyrimidin-3-yl)acetamide

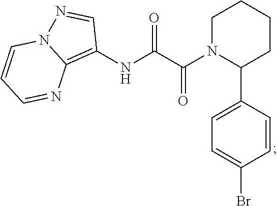 [SMILES: O=C(Nc1cnn2cccnc12)C(=O)N1CCCCC1c1ccc(Br)cc1]

d. 2-(2-(1H-pyrazol-4-yl)piperidin-1-yl)-N-(1H-indazol-5-yl)-2-oxoacetamide

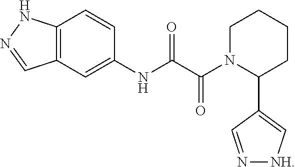 [SMILES: O=C(Nc1ccc2[nH]ncc2c1)C(=O)N1CCCCC1c1cn[nH]c1]

In one embodiment, provided herein are compounds of Formula (I):

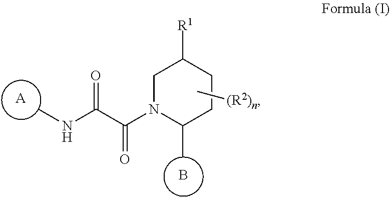

or pharmaceutically acceptable salts thereof,
wherein:
Ring A is an optionally substituted fused bicyclic 8-10 membered heteroaryl ring system containing at least one nitrogen atom, wherein the 8-10 membered refers to the total number of atoms in the fused system;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
each R^{a1} and R^{a2} is independently selected from the group consisting of H, —C₁-C₆ alkyl, —C₁-C₆ haloalkyl, —C₁-C₆ heteroalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
provided that the compound is not:
a. N-(8-fluoroquinolin-3-yl)-2-oxo-2-(2-phenylpiperidin-1-yl)acetamide;
b. 2-(2-(1H-pyrazol-4-yl)piperidin-1-yl)-N-(1H-indol-4-yl)-2-oxoacetamide;
c. 2-(4-methyl-2-(5-methylfuran-2-yl)piperidin-1-yl)-2-oxo-N-(4,5,6,7-tetrahydro-1H-indazol-7-yl)acetamide;
d. 2-(2-(1H-pyrazol-4-yl)piperidin-1-yl)-N-(1H-indazol-5-yl)-2-oxoacetamide.

In one embodiment, provided is a compound of Formula (I) and Formula (A):

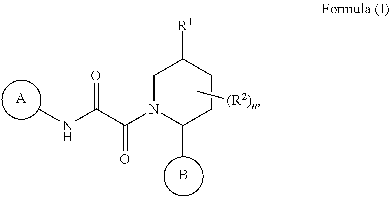

or a pharmaceutically acceptable salt thereof,
   wherein:
   Ring A is a fused bicyclic 8-10 membered heteroaryl ring containing at least one nitrogen atom substituted at any available positions with 0, 1, 2 or 3 instances of R⁴, wherein the 8-10 membered refers to the total number of atoms in the fused system;
   Ring B is selected from the group consisting of —C₃-C₁₀ carbocyclyl, 3-10 membered heterocyclyl, C₆-C₁₀ aryl and 5-10 membered heteroaryl, wherein each carbocyclyl, heterocyclyl, aryl and heteroaryl is substituted at any available position with 0, 1, 2 or 3 instances of R³;
   each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(—O)₂R^{a1} and —S(═O)₂N(R^{a1})₂;
   each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
   each R³ is independently selected from the group consisting of -D, —O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein each alkyl, cycloalkyl, heteroalkyl, heterocyclyl, aryl, heteroaryl, cycloalkylalkyl, heterocyclylalkyl, arylalkyl and heteroarylalkyl of R³ is substituted at any available position with 0, 1, 2, 3, 4, or 5 instances of R⁶;
   each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
   each R⁶ is independently selected from the group consisting of —C₁-C₆ alkyl, —C₃-C₉ cycloalkyl, 3-10 membered heterocyclyl, —OH, ═O, halo, —OC₁-C₆ alkyl, —C₁-C₆ haloalkyl, —C(═O)C₁-C₆ alkyl, —N(C₁-C₆ alkyl)₂, —C₁-C₆ heteroalkyl, and —NHC(═O)C₁-C₆ alkyl, wherein one or more hydrogens of the —C₁-C₆ alkyl can be replaced with deuterium;
   each R^{a1}, R^{a2}, R^{a3} and R^{a4} is independently selected from the group consisting of H, —C₁-C₆ alkyl, —C₁-C₆ haloalkyl, —C₁-C₆ heteroalkyl, —C₁-C₆ haloalkyl, C₃-C₉ cycloalkyl, 3-membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is substituted at any available position with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
   n is 0, 1, 2 or 3;
provided that the compound is not:
a. N-(8-fluoroquinolin-3-yl)-2-oxo-2-(2-phenylpiperidin-1-yl)acetamide;
b. 2-(2-(1H-pyrazol-4-yl)piperidin-1-yl)-N-(1H-indol-4-yl)-2-oxoacetamide;
c. 2-(4-methyl-2-(5-methylfuran-2-yl)piperidin-1-yl)-2-oxo-N-(4,5,6,7-tetrahydro-1H-indazol-7-yl)acetamide; or
d. 2-(2-(1H-pyrazol-4-yl)piperidin-1-yl)-N-(1H-indazol-5-yl)-2-oxoacetamide.

As generally defined herein, R^{B} is optionally substituted C₁-C₆ alkyl. In some embodiments, R^{B} is —C₁-C₆ alkyl substituted with 0, 1, 2 or 3 substituents independently selected from ═O, —F, —Cl, —OH—, —OMe, —NH₂ and —CN. In some embodiments, R^{B} is unsubstituted-C₁-C₆ alkyl. In some embodiments, R^{B} is selected from -Me, -Et-, -Pr, -ⁱPr and -^{t}Bu. In some embodiments, R^{B} is selected from -Me, -Et-, -ⁱPr and -^{t}Bu. In some embodiments, R¹ is not H and R^{B} and R¹ are in a trans relative configuration. In some embodiments, R¹ is not H and R^{B} and R¹ are in a cis relative configuration.

In some embodiments, R¹ is absent (e.g., if a spiro ring formed by two R² groups is attached to the atom that would otherwise bear R¹).

In certain embodiments, R¹ is not H and Ring B and R¹ are in a trans relative configuration. In other embodiments, R¹ is not H and Ring B and R¹ are in a cis relative configuration.

In some embodiments, the moiety represented as

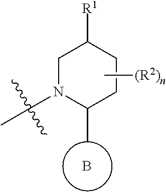

in Formula (I) is selected from the group consisting of:

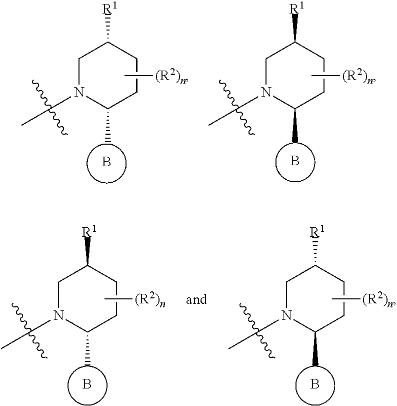

wherein Ring B, R¹, R² and n are as defined in any of the embodiments described herein.

In certain embodiments, the moiety represented as

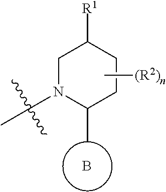

is selected from the group consisting of:

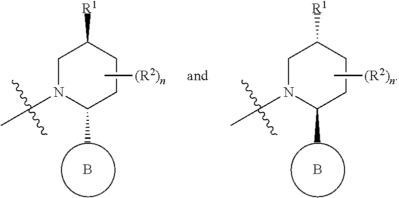

wherein Ring B, R¹, R² and n are as defined in any of the embodiments described herein.

In other embodiments, the moiety represented as

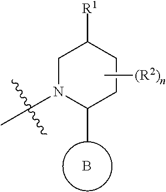

is selected from the group consisting of:

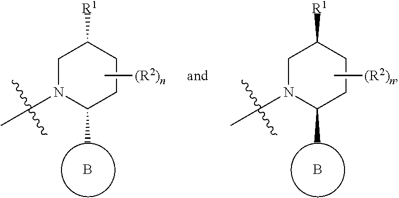

wherein Ring B, R¹, R² and n are as defined in any of the embodiments described herein.

In some embodiments, the compound of Formula (I) is of Formula (Ia)

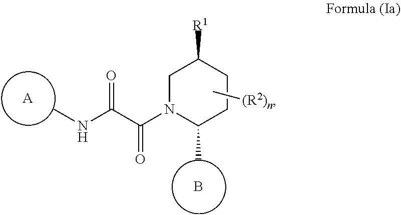

wherein Ring A, Ring B, R¹, R² and n are as defined in any of the embodiments described herein.

In some embodiments, the compound of Formula (I) is of Formula (Ib)

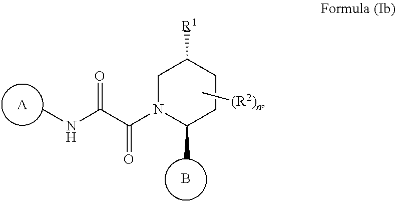

wherein Ring A, Ring B, R¹, R² and n are as defined in any of the embodiments described herein.

In some embodiments, the compound of Formula (I) is of Formula (Ic)

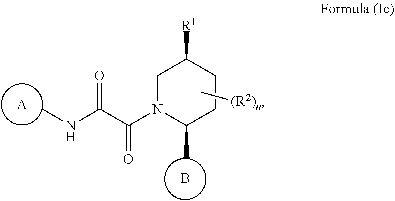

wherein Ring A, Ring B, R¹, R² and n are as defined in any of the embodiments described herein.

In some embodiments, the compound of Formula (I) is of Formula (Id)

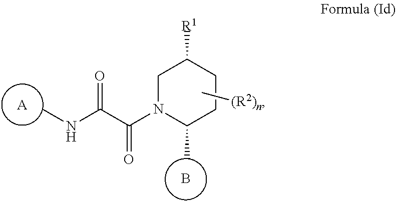

wherein Ring A, Ring B, R¹, R² and n are as defined in any of the embodiments described herein.

As generally defined herein, Ring A is an optionally substituted fused bicyclic 8-10 membered heteroaryl ring system containing at least one nitrogen atom, wherein the 8-10 membered refers to the total number of atoms in the fused system.

In some embodiments, Ring A is a fused bicyclic 8-10 membered heteroaryl ring containing at least one nitrogen atom, wherein Ring A is substituted at any available positions with 0, 1, 2 or 3 instances of R⁴, wherein R⁴ is as defined in any of the embodiments described herein, and wherein the 8-10 membered refers to the total number of atoms in the fused system.

In some embodiments, Ring A is an optionally substituted fused bicyclic 8-10 membered heteroaryl ring system containing at least one nitrogen atom and 0, 1, 2 or 3 additional heteroatoms selected from the group consisting of N, O and S or oxidized forms thereof, wherein the 8-10 membered refers to the total number of atoms in the fused system. In some embodiments, Ring A is substituted at any available positions with 0, 1, 2 or 3 instances of R⁴, wherein R⁴ is as defined in any of the embodiments described herein.

In some embodiments, Ring A contains a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), fused to a carbocyclyl or heterocyclyl ring, wherein the total number of atoms in the fused system is between 8 and 10 and the system contains a total of 2, 3 or 4 heteroatoms selected from the group consisting of N, O and S or oxidized forms thereof. The attachment point is on the heteroaryl ring.

In some embodiments, Ring A is substituted at any available positions with 0, 1, 2 or 3 instances of R⁴, wherein R⁴ is as defined in any of the embodiments described herein.

In some embodiments, Ring A contains a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), fused to a carbocyclyl ring, wherein the total number of atoms in the fused system is between 8 and 10. In some embodiments, Ring A is substituted at any available positions with 0, 1, 2 or 3 instances of R⁴, wherein R⁴ is as defined in any of the embodiments described herein.

In some embodiments, Ring A contains a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), fused to a heterocyclyl ring, wherein the total number of atoms in the fused system is between 8 and 10 and the system contains a total of 2, 3 or 4 heteroatoms selected from the group consisting of N, O and S or oxidized forms thereof. In some embodiments, Ring A is substituted at any available positions with 0, 1, 2 or 3 instances of R⁴, wherein R⁴ is as defined in any of the embodiments described herein.

In some embodiments, Ring A contains a phenyl ring fused with a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), wherein the attachment point is on either the phenyl or the heteroaryl ring. In some embodiments, Ring A is substituted at any available positions with 0, 1, 2 or 3 instances of R⁴, wherein R⁴ is as defined in any of the embodiments described herein.

In some embodiments, Ring A is selected from the group consisting of:

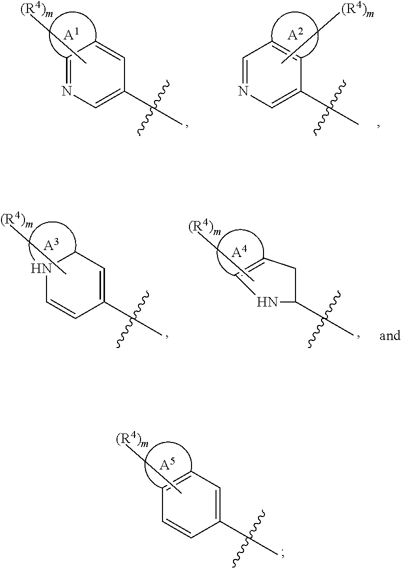

wherein:
each of rings A¹, A² and A⁴ is independently 4-6 membered carbocyclyl, 4-6 membered heterocyclyl, 5-6 membered heteroaryl or phenyl;
each rings A³ is independently a 4-6 membered heterocyclyl or 5-6 membered heteroaryl, wherein the heterocyclyl and heteroaryl contain at least one nitrogen atom;
each ring A⁵ is independently a 5-6 membered heteroaryl, wherein the heteroaryl contains at least one nitrogen atom;
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
m is 0, 1, 2 or 3.

In one embodiment, rings A¹, A², and A⁴ are each independently a 4-6 membered carbocyclyl, a 4-6 membered heterocyclyl containing 1, 2 or 3 heteroatoms selected from the group consisting of O, N, S or oxidized forms thereof, a 5-6 membered heteroaryl containing 1, 2 or 3 heteroatoms selected from the group consisting of O, N, S or oxidized forms thereof or a phenyl.

In one embodiment, each ring A³ is independently a 4-6 membered heterocyclyl or 5-6 membered heteroaryl, wherein the heterocyclyl and heteroaryl contain at least one nitrogen atom and 0, 1 or 2 additional heteroatoms selected from the group consisting of N, O or S or oxidized forms thereof.

In one embodiment, each ring A⁵ is independently a 5-6 membered heteroaryl, wherein the heteroaryl contains at least one nitrogen atom and 0, 1 or 2 additional heteroatoms selected from the group consisting of N, O or S or oxidized forms thereof.

In one embodiment, Ring A is

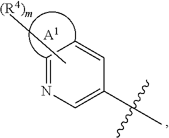

wherein A¹, R⁴ and m are as defined in any of the embodiments described herein.

In one embodiment, ring A is

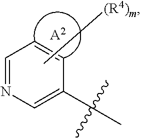

wherein A², R⁴ and m are as defined in any of the embodiments described herein.

In one embodiment, ring A

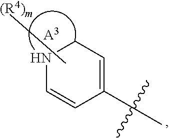

is wherein A³, R⁴ and m are as defined in any of the embodiments described herein.

In one embodiment, ring A

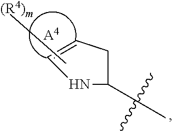

is wherein A⁴, R⁴ and m are as defined in any of the embodiments described herein.

In one embodiment, ring A

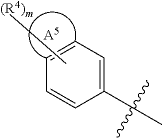

is wherein A⁵, R⁴ and m are as defined in any of the embodiments described herein.

In some embodiments, Ring A is selected from the group consisting of:

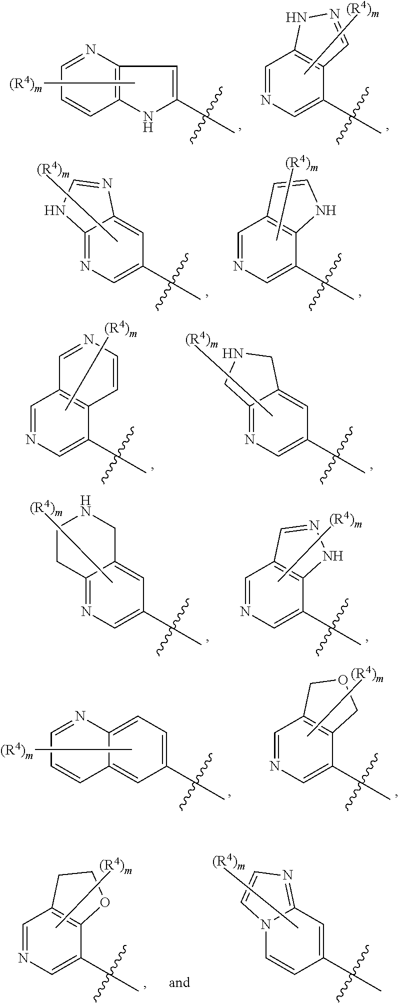

wherein R⁴ and m are as defined in any of the embodiments described herein.

In some embodiments, Ring A is selected from the group consisting of:

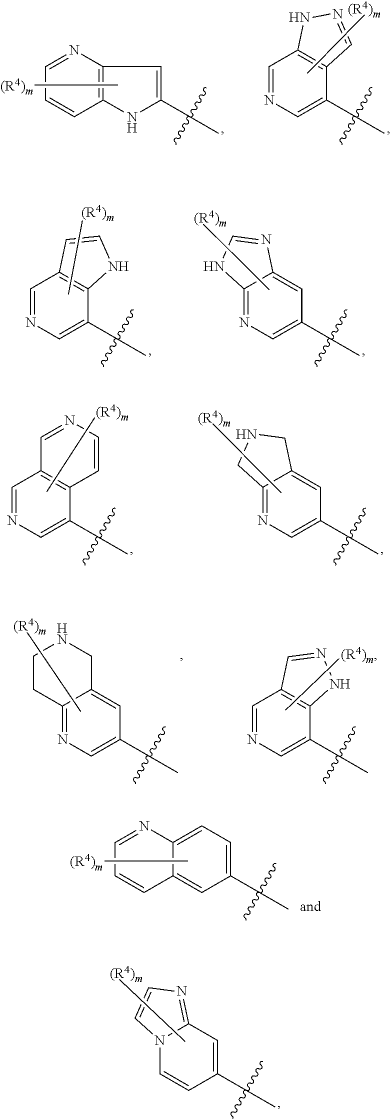

wherein R⁴ and m are as defined in any of the embodiments described herein.

In some embodiments, Ring A is selected from the group consisting of:

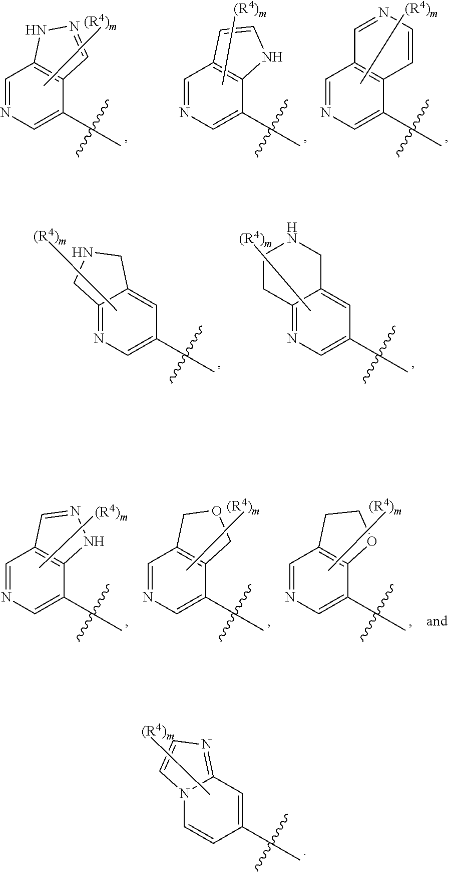

In some embodiments, Ring A is selected from the group consisting of:

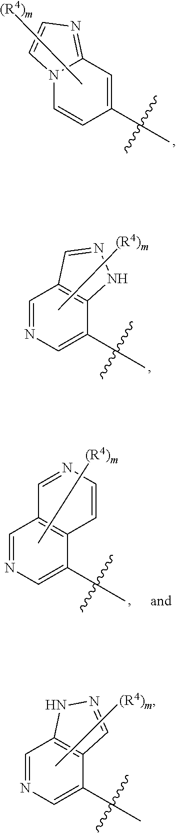

wherein R⁴ and m are as defined in any of the embodiments described herein.

In some embodiments, Ring A is selected from the group consisting of:

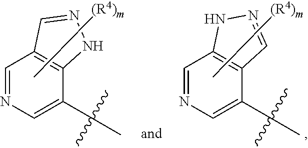

wherein R⁴ and m are as defined in any of the embodiments described herein.

In some embodiments, Ring A is selected from the group consisting of:

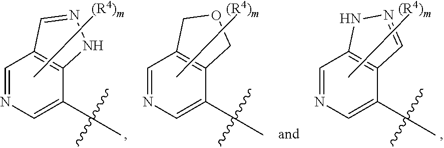

wherein R⁴ and m are as defined in any of the embodiments described herein.

In certain embodiments, Ring A is selected from the group consisting of:

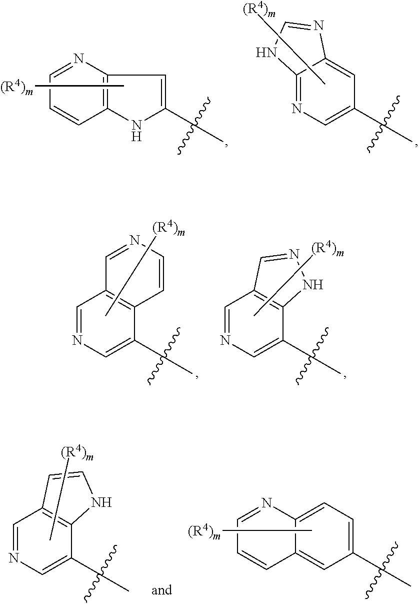

wherein R⁴ and m are as defined in any of the embodiments described herein.

In some embodiments, ring A is:

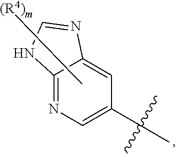

wherein R⁴ and m are as defined in any of the embodiments described herein.

In some embodiments, ring A is:

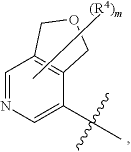

wherein R⁴ and m are as defined in any of the embodiments described herein.

In some embodiments, ring A is:

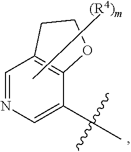

wherein R⁴ and m are as defined in any of the embodiments described herein.

In some embodiments, ring A is:

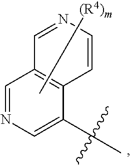

wherein R⁴ and m are as defined in any of the embodiments described herein.

In some embodiments, ring A is:

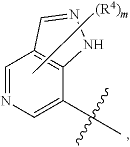

wherein R⁴ and m are as defined in any of the embodiments described herein.

In some embodiments, ring A is:

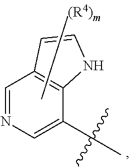

wherein R⁴ and m are as defined in any of the embodiments described herein.

In some embodiments, ring A is:

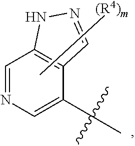

wherein R⁴ and m are as defined in any of the embodiments described herein.

In some embodiments, ring A is:

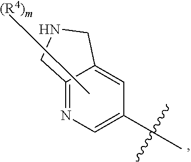

wherein R⁴ and m are as defined in any of the embodiments described herein.

In some embodiments, ring A is:

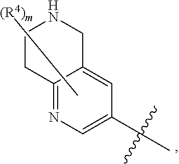

wherein R⁴ and m are as defined in any of the embodiments described herein.

In some embodiments, ring A is:

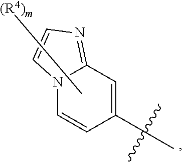

wherein R⁴ and m are as defined in any of the embodiments described herein.

In some embodiments, ring A is:

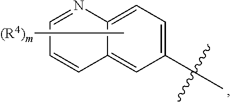

wherein R⁴ and m are as defined in any of the embodiments described herein.

In some embodiments, ring A is:

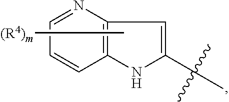

wherein R⁴ and m are as defined in any of the embodiments described herein.

As generally defined herein, m is 0, 1, 2 or 3. In some embodiments, m is 0, 1 or 2.

In some embodiments, m is 0 or 1. In some embodiments, m is 1 or 2.

In some embodiments, m is 0.

In some embodiments, m is 1.

In some embodiments, m is 2.

In some embodiments, m is 3.

In some embodiments, Ring A is selected from the group consisting of:

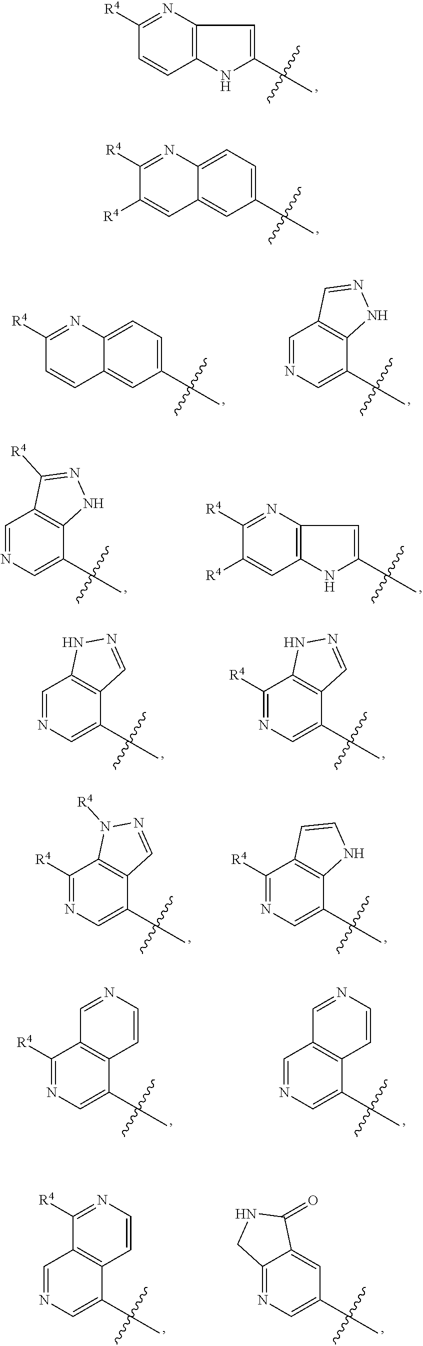

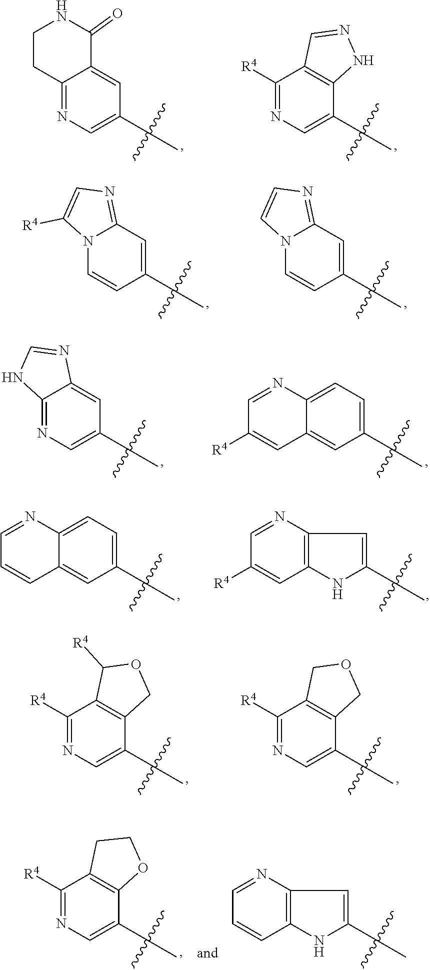

wherein R⁴ is as defined in any of the embodiments described herein.

In some embodiments, Ring A is selected from the group consisting of:

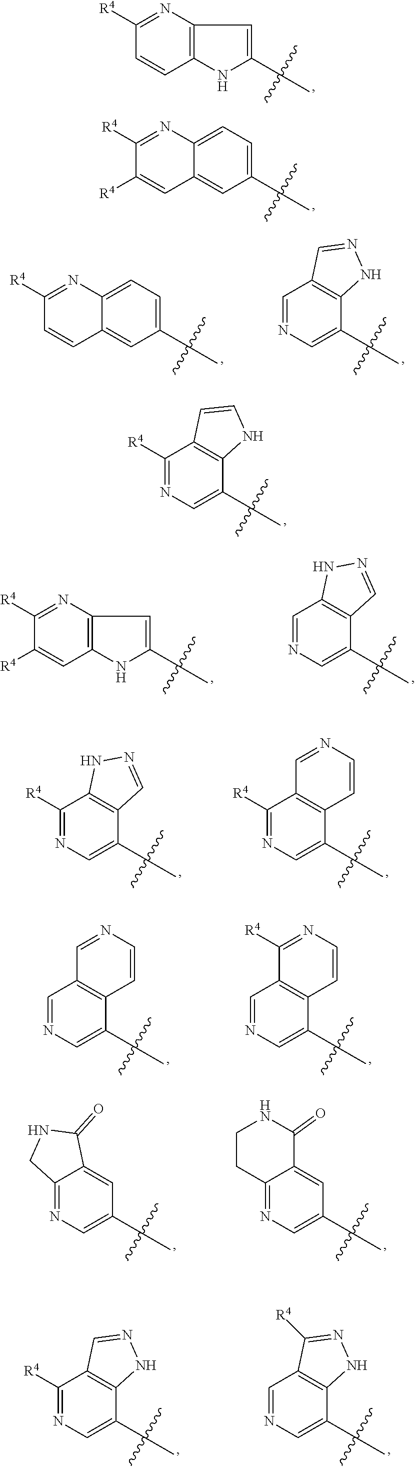

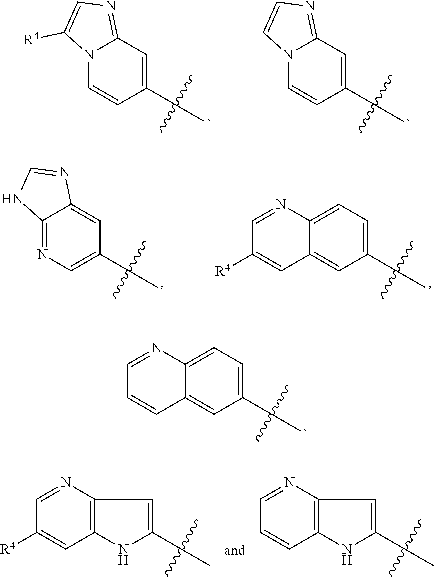

wherein R⁴ is as defined in any of the embodiments described herein.

In some embodiments, Ring A is selected from the group consisting of:

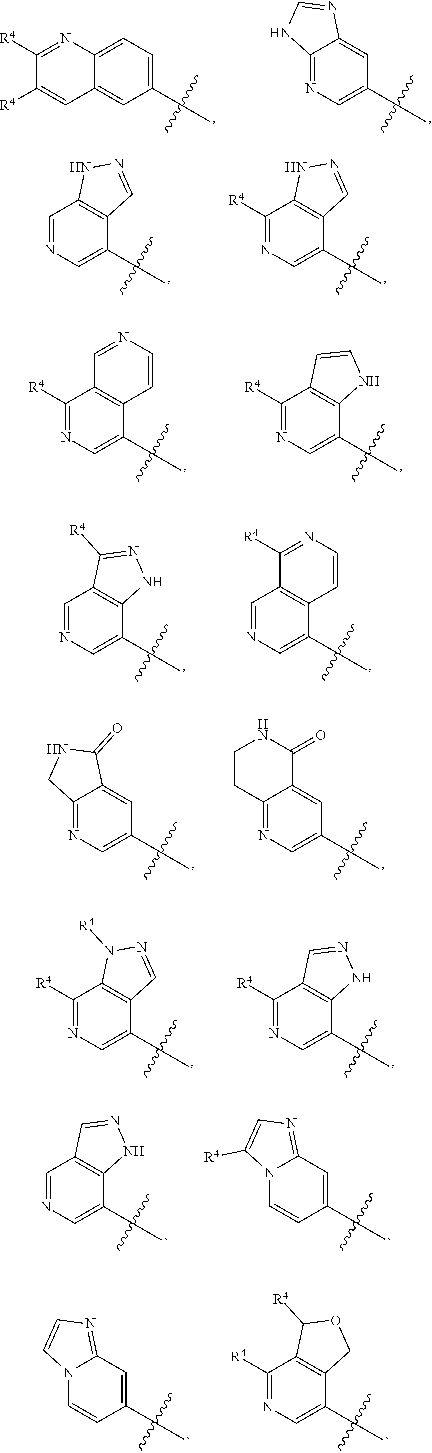

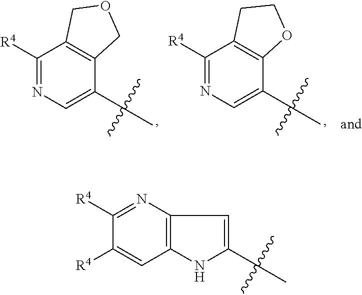

wherein R⁴ is as defined in any of the embodiments described herein.

In some embodiments, Ring A is selected from the group consisting of:

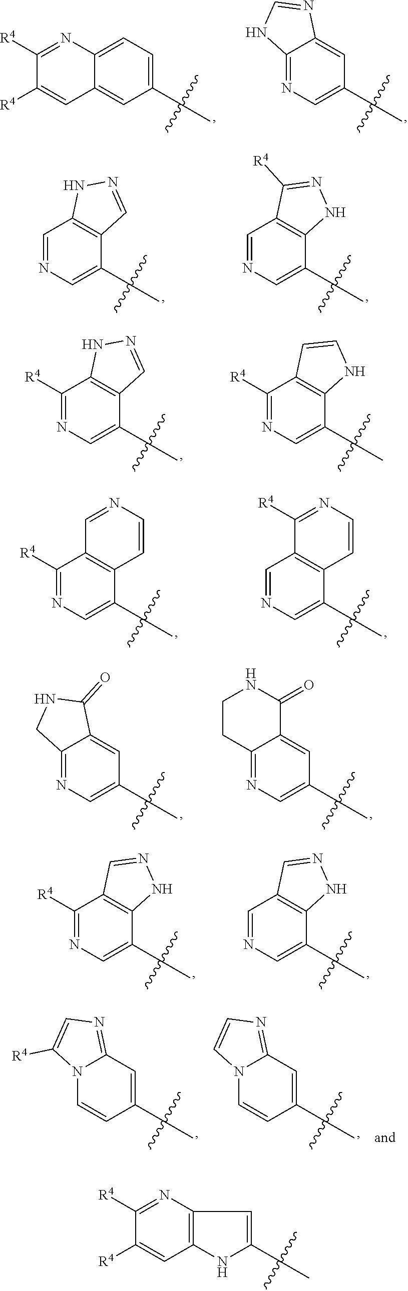

wherein R⁴ is as defined in any of the embodiments described herein.

In some embodiments, Ring A is selected from the group consisting of:

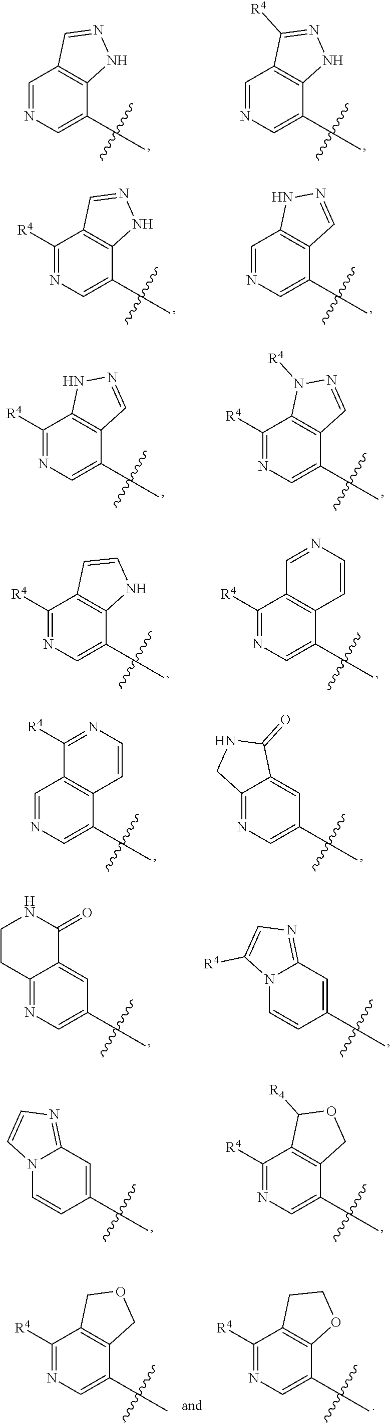

In some embodiments, Ring A is

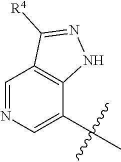

wherein R⁴ is as defined in any of the embodiments described herein. In some embodiments, Ring A is

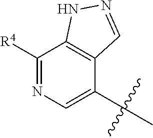

wherein R⁴ is as defined in any of the embodiments described herein. In some embodiments, Ring A is

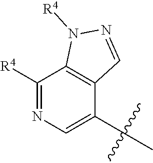

wherein R⁴ is as defined in any of the embodiments described herein. In some embodiments, Ring A is

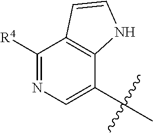

wherein R⁴ is as defined in any of the embodiments described herein. In some embodiments, Ring A is

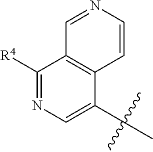

wherein R⁴ is as defined in any of the embodiments described herein. In some embodiments, Ring A is

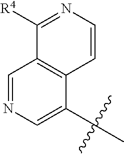

wherein R⁴ is as defined in any of the embodiments described herein, wherein R⁴ is as defined in any of the embodiments described herein. In some embodiments, Ring A is

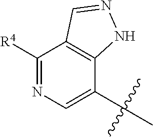

wherein R⁴ is as defined in any of the embodiments described herein. In some embodiments, Ring A is

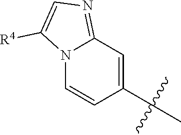

wherein R⁴ is as defined in any of the embodiments described herein.

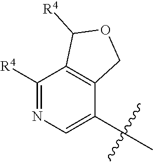

In some embodiments, Ring A is wherein R⁴ is as defined in any of the embodiments described herein. In some embodiments, Ring A is

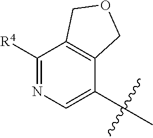

wherein R⁴ is as defined in any of the embodiments described herein. In some embodiments, Ring A is wherein

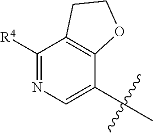

R⁴ is as defined in any of the embodiments described herein. In some embodiments, Ring A is

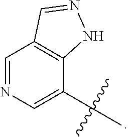 [SMILES: *c1cncc2cn[nH]c12]

In some embodiments, Ring A is

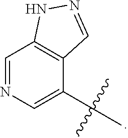 [SMILES: *c1cncc2[nH]ncc12]

In some embodiments, Ring A is

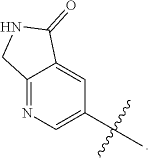 [SMILES: *c1cnc2c(c1)C(=O)NC2]

In some embodiments, Ring A is

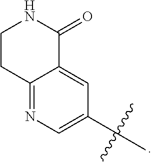 [SMILES: *c1cnc2c(c1)C(=O)NCC2]

In some embodiments, Ring A is

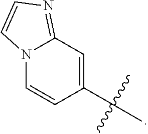 [SMILES: *c1ccn2ccnc2c1]

In some embodiments, Ring A is selected from the group consisting of:

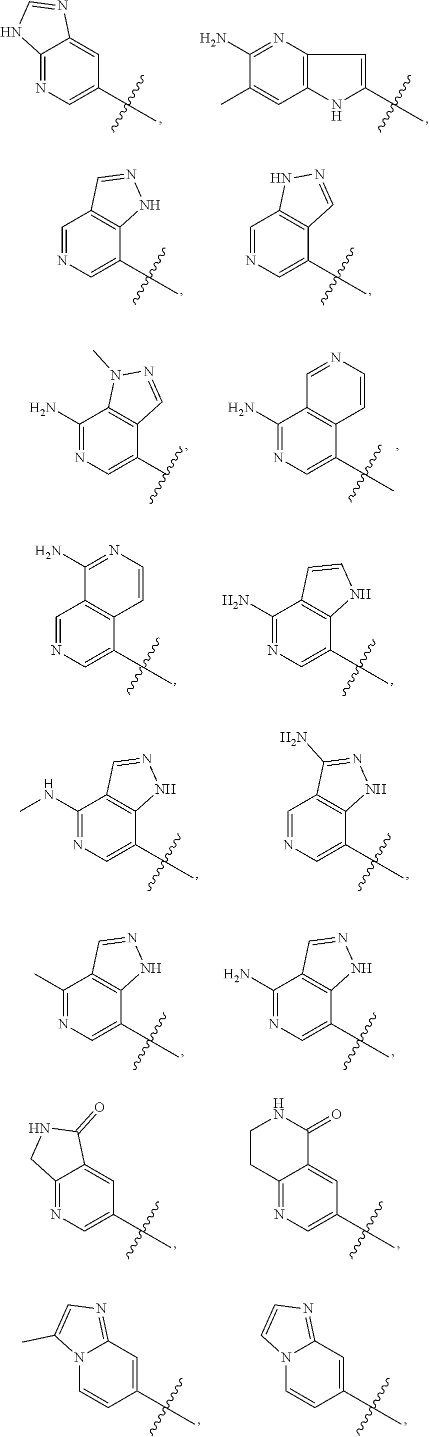

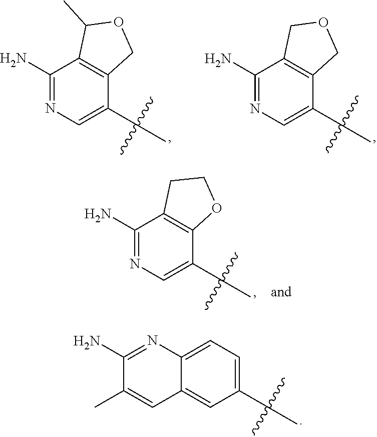 [SMILES: *c1cnc(N)c2c1COC2C ; *c1cnc(N)c2c1COC2 ; *c1cnc(N)c2c1OCC2 ; *c1ccc2nc(N)c(C)cc2c1]

In some embodiments, Ring A is selected from the group consisting of:

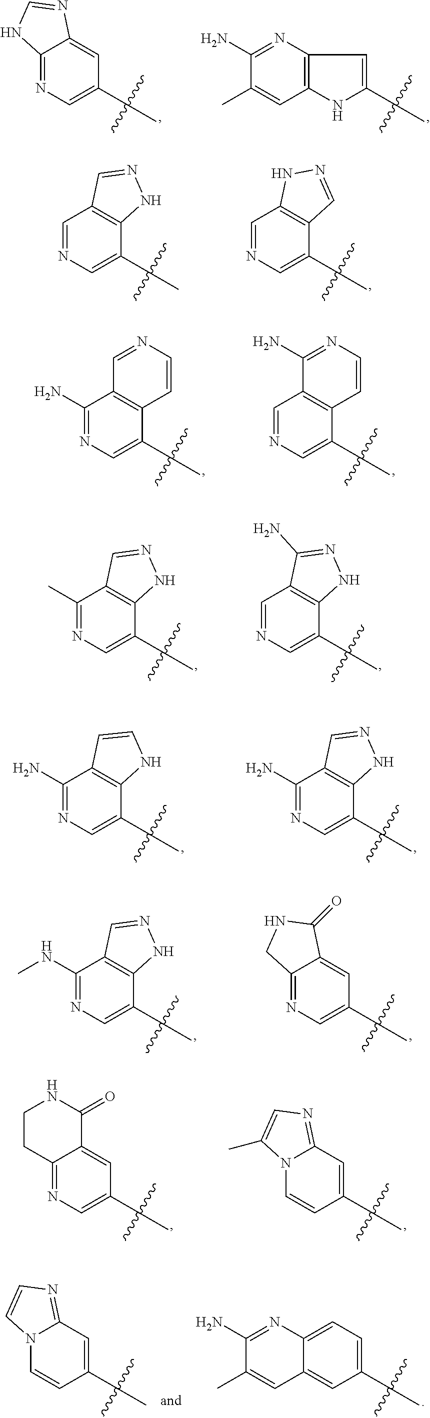

In some embodiments, Ring A is selected from the group consisting of:

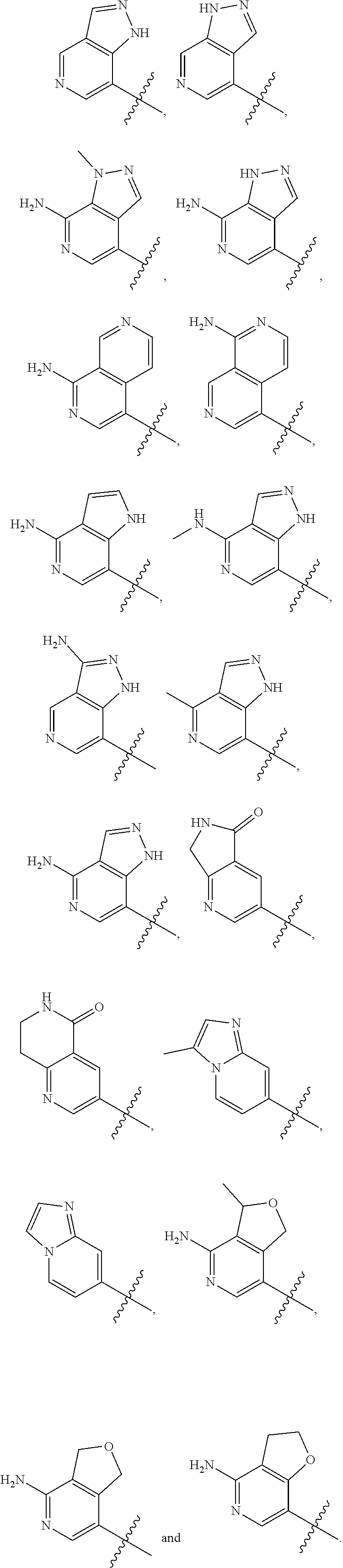

In some embodiments, Ring A is selected from the group consisting of:

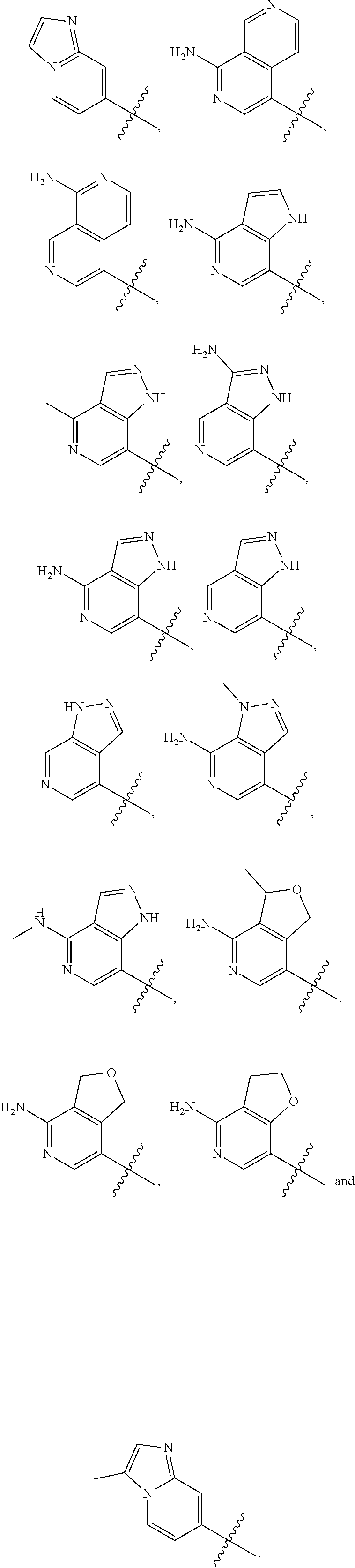

In some embodiments, Ring A is selected from the group consisting of:

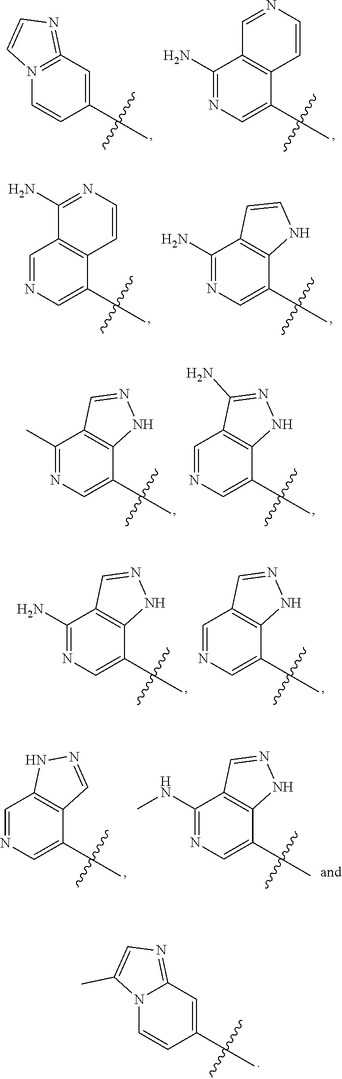

In some embodiments, Ring A is selected from the group consisting of:

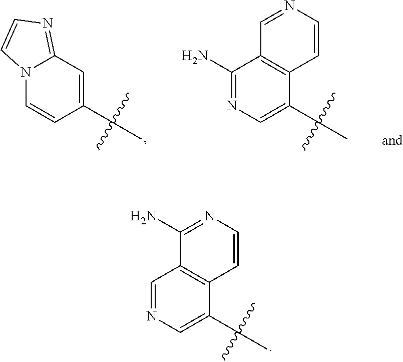 [SMILES: *c1ccn2ccnc2c1 ; *c1cnc(N)c2cnccc12 ; *c1cncc2c(N)nccc12]

In certain embodiments, Ring A is selected from the group consisting of:

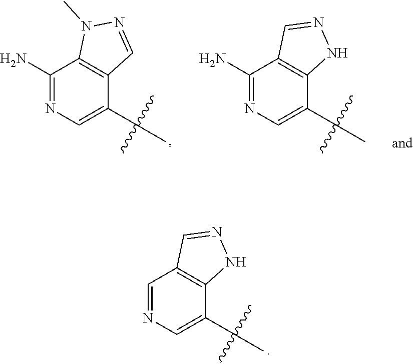 [SMILES: *c1cnc(N)c2c1cnn2C ; *c1cnc(N)c2cn[nH]c12 ; *c1cncc2cn[nH]c12]

In certain embodiments, Ring A is selected from the group consisting of:

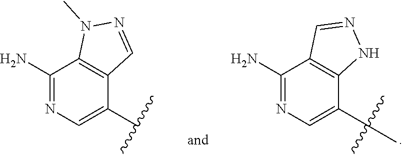 [SMILES: *c1cnc(N)c2c1cnn2C ; *c1cnc(N)c2cn[nH]c12]

In certain embodiments, Ring A is selected from the group consisting of:

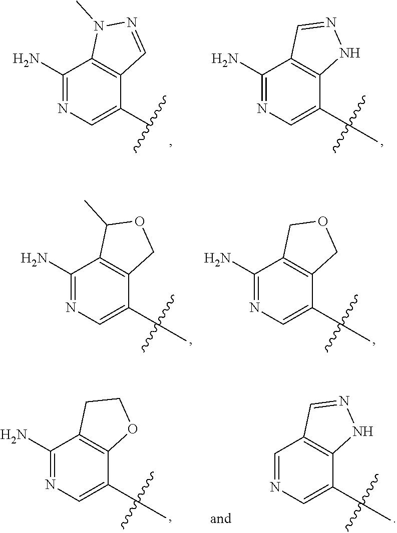 [SMILES: *c1cnc(N)c2c1cnn2C ; *c1cnc(N)c2cn[nH]c12 ; *c1cnc(N)c2c1COC2C ; *c1cnc(N)c2c1COC2 ; *c1cnc(N)c2c1OCC2 ; *c1cncc2cn[nH]c12]

In certain embodiments, Ring A is selected from the group consisting of:

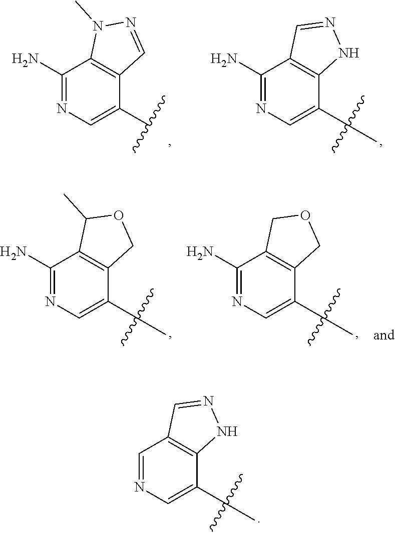 [SMILES: *c1cnc(N)c2c1cnn2C ; *c1cnc(N)c2cn[nH]c12 ; *c1cnc(N)c2c1COC2C ; *c1cnc(N)c2c1COC2 ; *c1cncc2cn[nH]c12]

In certain embodiments, Ring A is selected from the group consisting of:

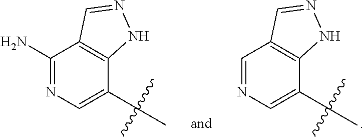 [SMILES: *c1cnc(N)c2cn[nH]c12 ; *c1cncc2cn[nH]c12]

In some embodiments, Ring A is

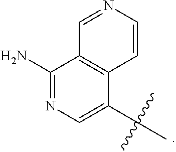 [SMILES: *c1cnc(N)c2cnccc12]

In some embodiments, Ring A is

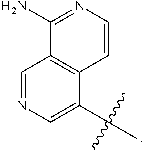 [SMILES: *c1cncc2c(N)nccc12]

In some embodiments, Ring A is

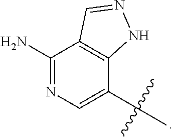 [SMILES: *c1cnc(N)c2cn[nH]c12]

In some embodiments, Ring A is

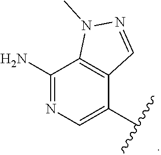 [SMILES: *c1cnc(N)c2c1cnn2C]

In some embodiments, Ring A is

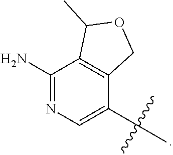 [SMILES: *c1cnc(N)c2c1COC2C]

In some embodiments, Ring A is

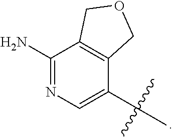 [SMILES: *c1cnc(N)c2c1COC2]

In some embodiments, Ring A is

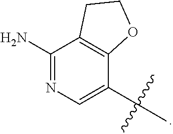 [SMILES: *c1cnc(N)c2c1OCC2]

In some embodiments, Ring A is

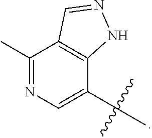 [SMILES: *c1cnc(C)c2cn[nH]c12]

In some embodiments, Ring A is

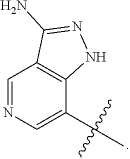 [SMILES: *c1cncc2c(N)n[nH]c12]

In some embodiments, Ring A is

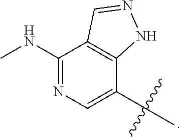 [SMILES: *c1cnc(NC)c2cn[nH]c12]

In some embodiments, Ring A is:

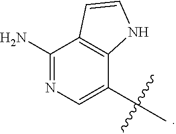 [SMILES: *c1cnc(N)c2cc[nH]c12]

In some embodiments, Ring A is

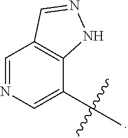 [SMILES: *c1cncc2cn[nH]c12]

In some embodiments, Ring A is:

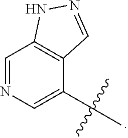 [SMILES: *c1cncc2[nH]ncc12]

In some embodiments, Ring A is

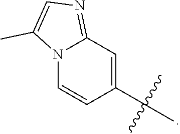 [SMILES: *c1ccn2c(C)cnc2c1]

In some embodiments, Ring A is:

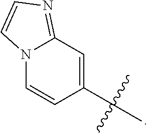 [SMILES: *c1ccn2ccnc2c1]

In some embodiments the compounds of Formula (I) are of Formula (II1):

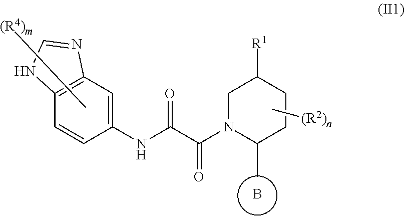

wherein Ring B, R¹, R², R⁴, m and n are as defined in any of the embodiments described herein. In some embodiments, the compounds are of Formula (II1a):

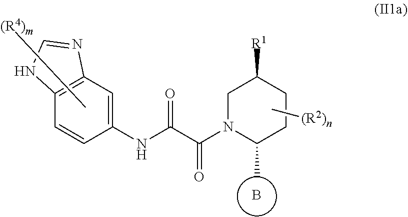

wherein Ring B, R¹, R², R⁴, m and n are as defined in any of the embodiments described herein.

In some embodiments the compounds of Formula (I) are of Formula (II2):

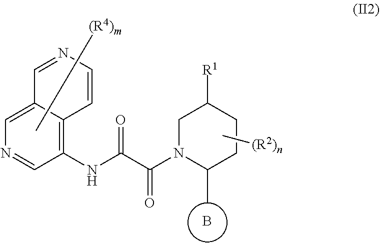

wherein Ring B, R¹, R², R⁴, m and n are as defined in any of the embodiments described herein.

In some embodiments, the compounds are of Formula (II2a):

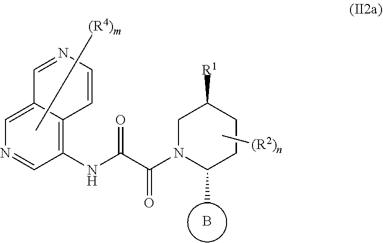

wherein Ring B, R¹, R², R⁴, m and n are as defined in any of the embodiments described herein.

In yet some embodiments, the compounds are of Formula (II2b):

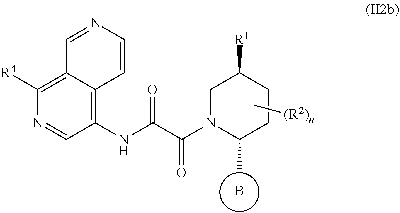

wherein Ring B, R¹, R², and n are as defined in any of the embodiments described herein.

In yet some embodiments, the compounds are of Formula (II2c):

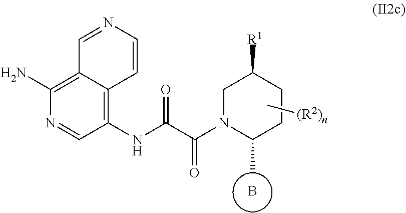

wherein Ring B, R¹, R², and n are as defined in any of the embodiments described herein.

In other embodiments, the compounds are of Formula (II2d):

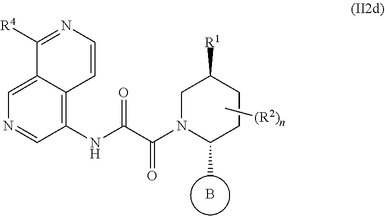

wherein Ring B, R¹, R², and n are as defined in any of the embodiments described herein.

In other embodiments, the compounds are of Formula (II2e):

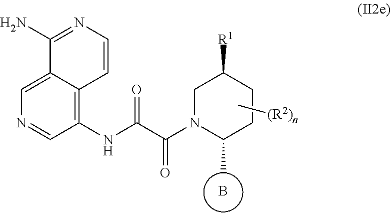

wherein Ring B, R¹, R², and n are as defined in any of the embodiments described herein.

In some embodiments the compounds of Formula (I) are of Formula (II3):

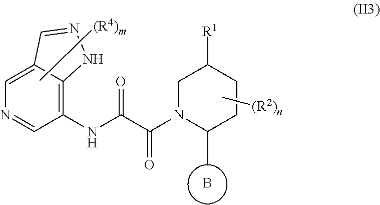

wherein Ring B, R¹, R², R⁴, m and n are as defined in any of the embodiments described herein.

In some embodiments, the compounds are of Formula (II3a):

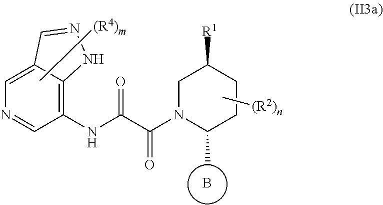

wherein Ring B, R¹, R², R⁴, m and n are as defined in any of the embodiments described herein.

In some embodiments, the compounds are of Formula (II3b):

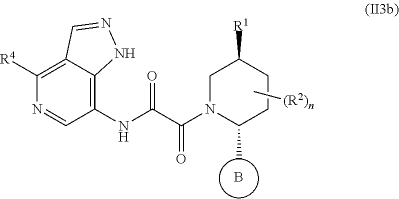

wherein Ring B, R¹, R² and n are as defined in any of the embodiments described herein.

In some embodiments, the compounds are of Formula (II3c):

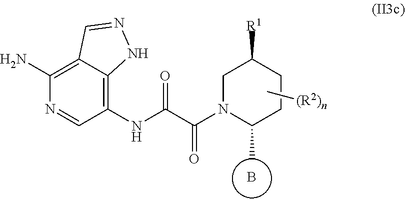

wherein Ring B, R¹, R² and n are as defined in any of the embodiments described herein.

In some embodiments, the compounds are of Formula (II3d):

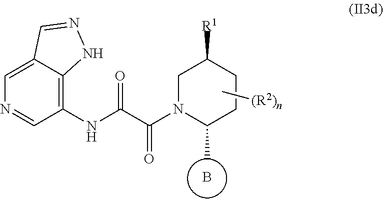

wherein Ring B, R¹, R² and n are as defined in any of the embodiments described herein.

In some embodiments, the compounds are of Formula (II3e):

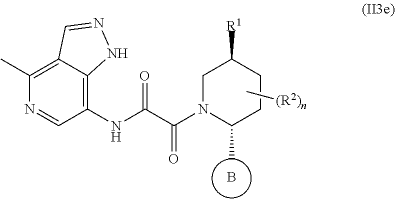

wherein Ring B, R¹, R² and n are as defined in any of the embodiments described herein.

In some embodiments, the compounds are of Formula (II3f):

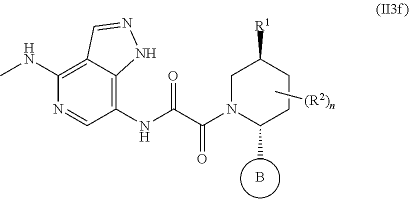

wherein Ring B, R¹, R² and n are as defined in any of the embodiments described herein.

In some embodiments, the compounds are of Formula (II3g):

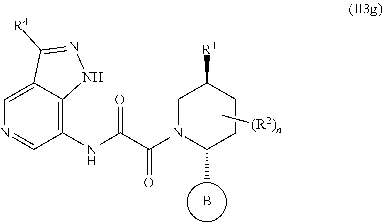

wherein Ring B, R¹, R² and n are as defined in any of the embodiments described herein.

In some embodiments, the compounds are of Formula (II3h):

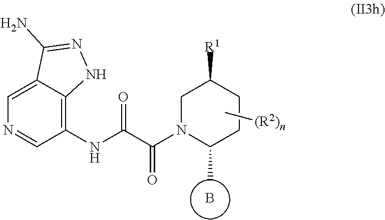

wherein Ring B, R¹, R² and n are as defined in any of the embodiments described herein.

In some embodiments the compounds of Formula (I) are of Formula (II4):

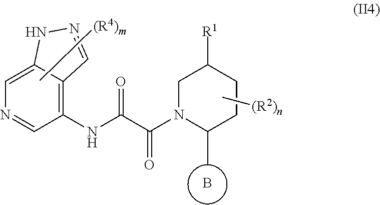

wherein Ring B, R¹, R², R⁴, m and n are as defined in any of the embodiments described herein.

In some embodiments, the compounds are of Formula (II4a):

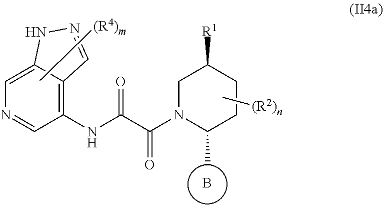

wherein Ring B, R¹, R², R⁴, m and n are as defined in any of the embodiments described herein.

In some embodiments, the compounds are of Formula (II4b):

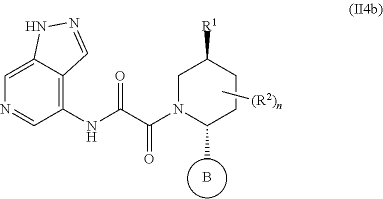

wherein Ring B, R¹, R² and n are as defined in any of the embodiments described herein.

In some embodiments, the compounds are of Formula (II4c):

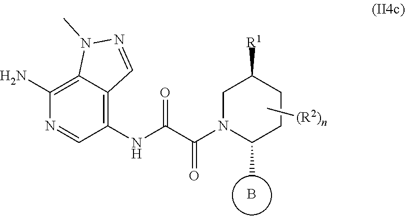

wherein Ring B, R¹, R² and n are as defined in any of the embodiments described herein.

In some embodiments the compounds of Formula (I) are of Formula (II5):

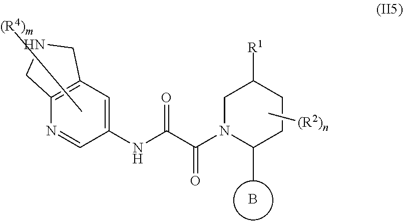

wherein Ring B, R¹, R², R⁴, m and n are as defined in any of the embodiments described herein.

In some embodiments, the compounds are of Formula (II5a):

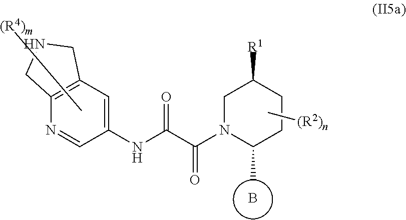

wherein Ring B, R¹, R², R⁴, m and n are as defined in any of the embodiments described herein.

In some embodiments the compounds of Formula (I) are of Formula (II6):

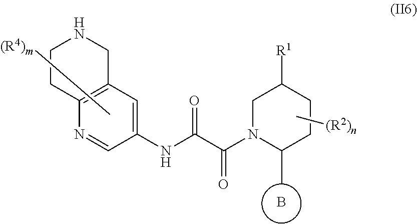

wherein Ring B, R¹, R², R⁴, m and n are as defined in any of the embodiments described herein.

In some embodiments, the compounds are of Formula (II6a):

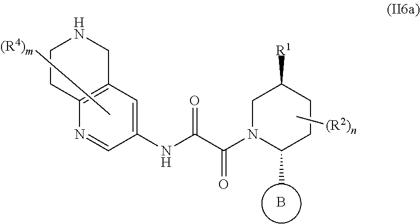

wherein Ring B, R¹, R², R⁴, m and n are as defined in any of the embodiments described herein.

In some embodiments the compounds of Formula (I) are of Formula (II7):

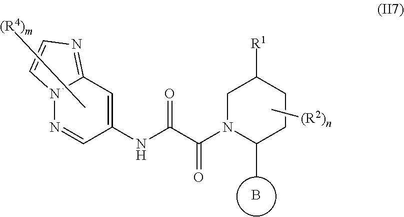

wherein Ring B, R¹, R², R⁴, m and n are as defined in any of the embodiments described herein.

In some embodiments, the compounds are of Formula (II7a):

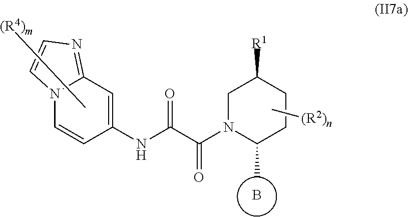

wherein Ring B, R¹, R², R⁴, m and n are as defined in any of the embodiments described herein.

In some embodiments, the compounds are of Formula (II7b):

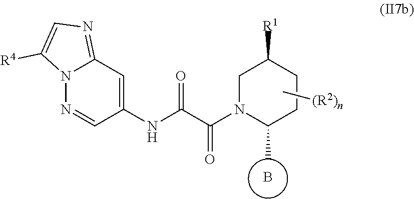

wherein Ring B, R¹, R², R⁴, m and n are as defined in any of the embodiments described herein.

In yet some embodiments, the compounds are of Formula (II7c):

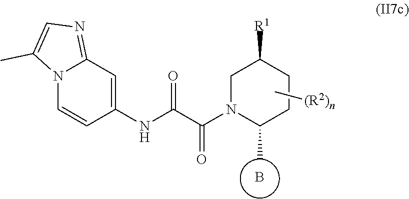

wherein Ring B, R¹, R², R⁴, m and n are as defined in any of the embodiments described herein.

In other embodiments, the compounds are of Formula (II7d):

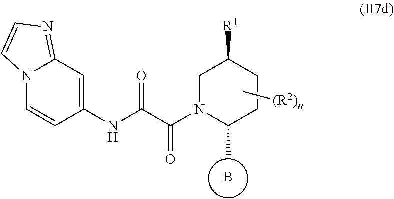

wherein Ring B, R¹, R², R⁴, m and n are as defined in any of the embodiments described herein.

In some embodiments the compounds of Formula (I) are of Formula (II8):

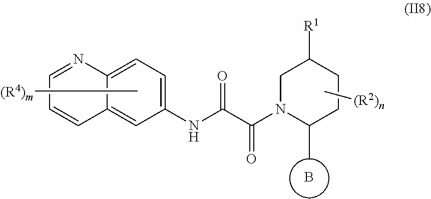

wherein Ring B, R¹, R², R⁴, m and n are as defined in any of the embodiments described herein.

In some embodiments, the compounds are of Formula (II8a):

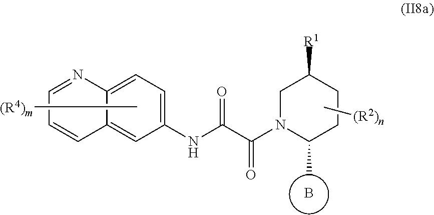

wherein Ring B, R¹, R², R⁴, m and n are as defined in any of the embodiments described herein.

In some embodiments the compounds of Formula (I) are of Formula (II9):

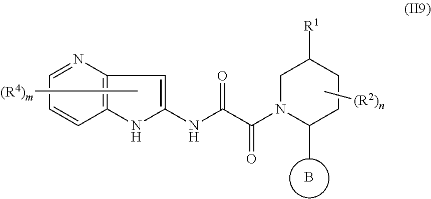

wherein Ring B, R¹, R², R⁴, m and n are as defined in any of the embodiments described herein.

In some embodiments, the compounds are of Formula (II9a):

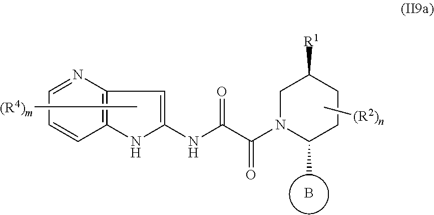

wherein Ring B, R¹, R², R⁴, m and n are as defined in any of the embodiments described herein.

In some embodiments the compounds of Formula (I) are of Formula (II10):

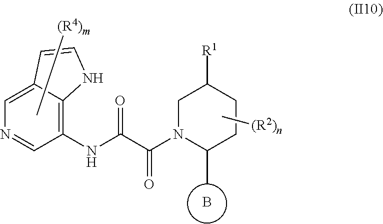

wherein Ring B, R¹, R², R⁴, m and n are as defined in any of the embodiments described herein.

In some embodiments, the compounds are of Formula (II10a):

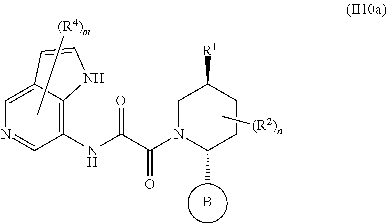

wherein Ring B, R¹, R², R⁴, m and n are as defined in any of the embodiments described herein.

In some embodiments, the compounds are of Formula (II10b):

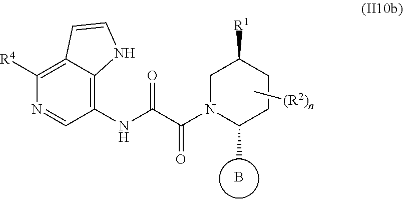

wherein Ring B, R¹, R², R⁴, m and n are as defined in any of the embodiments described herein.

In some embodiments, the compounds are of Formula (II10c):

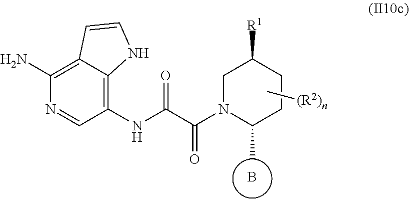

wherein Ring B, R¹, R², R⁴, m and n are as defined in any of the embodiments described herein.

In some embodiments the compounds of Formula (I) are of Formula (II11):

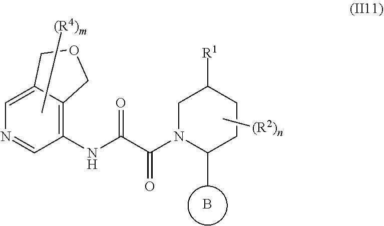

wherein Ring B, R¹, R², R⁴, m and n are as defined in any of the embodiments described herein.

In some embodiments, the compounds are of Formula (III1a):

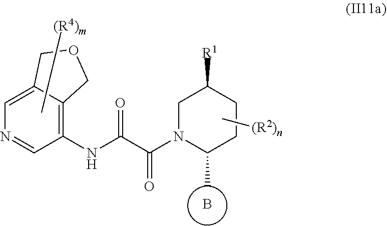

wherein Ring B, R¹, R², R⁴, m and n are as defined in any of the embodiments described herein.

In some embodiments, the compounds are of Formula (II11b):

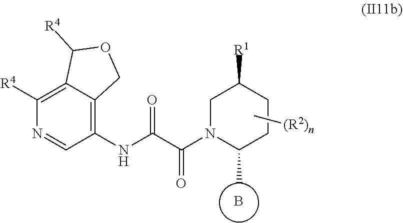

wherein Ring B, R¹, R², R⁴, m and n are as defined in any of the embodiments described herein.

In some embodiments, the compounds are of Formula (II11c):

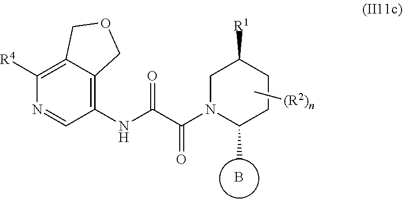

wherein Ring B, R¹, R², R⁴, m and n are as defined in any of the embodiments described herein.

In some embodiments, the compounds are of Formula (II11d):

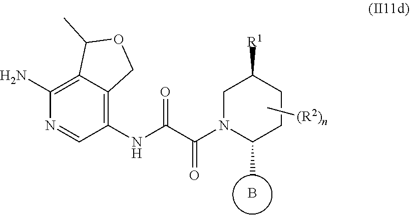

wherein Ring B, R¹, R², R⁴, m and n are as defined in any of the embodiments described herein.

In some embodiments, the compounds are of Formula (II11e):

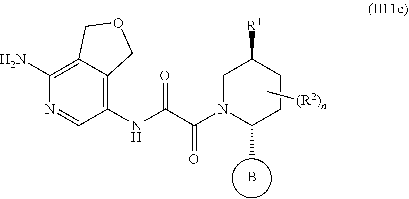

wherein Ring B, R¹, R², R⁴, m and n are as defined in any of the embodiments described herein.

In some embodiments the compounds of Formula (I) are of Formula (II12):

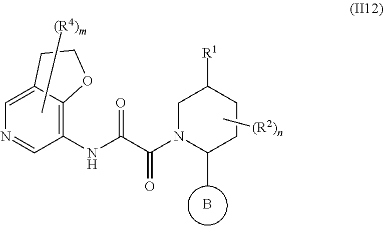

wherein Ring B, R¹, R², R⁴, m and n are as defined in any of the embodiments described herein.

In some embodiments, the compounds are of Formula (II12a):

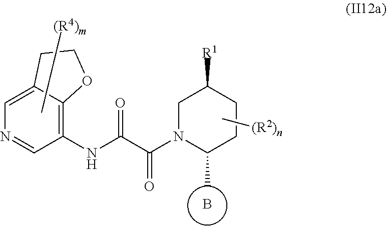

wherein Ring B, R¹, R², R⁴, m and n are as defined in any of the embodiments described herein.

In some embodiments, the compounds are of Formula (II12b):

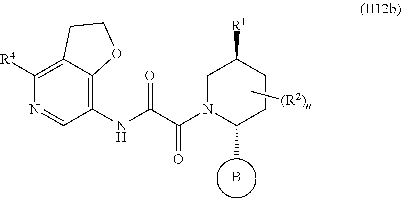

wherein Ring B, R¹, R², R⁴, m and n are as defined in any of the embodiments described herein.

In some embodiments, the compounds are of Formula (II12c):

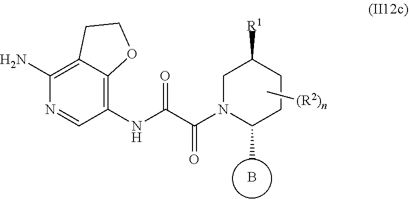

wherein Ring B, R¹, R², R⁴, m and n are as defined in any of the embodiments described herein.

As generally defined herein, each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂, wherein R^{a4} is as defined in any of the embodiments described herein.

In some embodiments, each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂, wherein R^{a4} is as defined in any of the embodiments described herein.

In some embodiments, R⁴ is selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂, wherein R^{a4} is as defined in any of the embodiments described herein.

In some embodiments, R⁴ is selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂, wherein R^{a4} is as defined in any of the embodiments described herein.

In certain embodiments, R⁴ is selected from the group consisting of -D, halo, ═O, —CN, —C₁-C₆ alkyl, —C₁-C₆ heteroalkyl, —C₁-C₆ haloalkyl, —C₃-C₉ cycloalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂, wherein R^{a4} is as defined in any of the embodiments described herein.

In certain embodiments, R⁴ is selected from the group consisting of halo, ═O, —CN, —C₁-C₆ alkyl, —C₁-C₆ heteroalkyl, —C₁-C₆ haloalkyl, —C₃-C₉ cycloalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂, wherein R^{a4} is as defined in any of the embodiments described herein.

In some embodiments, R⁴ is selected from the group consisting of -D, ═O, —C₁-C₆ alkyl, —C₁-C₆ haloalkyl, —C₃-C₉ cycloalkyl, —OR^{a4} and —N(R^{a4})₂, wherein R^{a4} is as defined in any of the embodiments described herein.

In some embodiments, R⁴ is selected from the group consisting of ═O, —C₁-C₆ alkyl, —C₁-C₆ haloalkyl, —C₃-C₉ cycloalkyl, —OR^{a4} and —N(R^{a4})₂, wherein R^{a4} is as defined in any of the embodiments described herein.

In some embodiments, R⁴ is selected from the group consisting of -D, ═O, —C₁-C₆ alkyl and —N(R^{a4})₂, wherein R^{a4} is as defined in any of the embodiments described herein.

In some embodiments, R⁴ is selected from the group consisting of ═O, —C₁-C₆ alkyl and —N(R^{a4})₂, wherein R^{a4} is as defined in any of the embodiments described herein.

In certain embodiments, R⁴ is selected from the group consisting of -D, ═O, -Me, -Et, -ⁱPr, -^{t}Bu, —NH₂, —NHCH₃ and —NH(CH₃)₂. In certain embodiments, R⁴ is selected from the group consisting of ═O, -Me, -Et, -ⁱPr, -^{t}Bu, —NH₂, —NHCH₃ and —NH(CH₃)₂. In some embodiments, R⁴ is selected from the group consisting of —NHCH₃, —NH₂ or -Me. In some embodiments, R⁴ is selected from the group consisting of —NH₂ or -Me.

In some embodiments, R⁴ is ═O.

In some embodiments, R⁴ is -D.

In certain embodiments, R⁴ is halo (e.g., fluoro, chloro, bromo, iodo). In some embodiments, R⁴ is —Cl. In some embodiments, R⁴ is —F. In some embodiments, R⁴ is —Br.

In some embodiments, R⁴ is —I.

In some embodiments, R⁴ is —CN.

In certain embodiments, R⁴ is —C₁-C₆ alkyl. In some embodiments, R⁴ is -Me. In some embodiments, R⁴ is -Et. In some embodiments R⁴ is -Pr or -iPr.

In some embodiments, R⁴ is —C₁-C₆ heteroalkyl. In some embodiments, R⁴ is methoxymethyl (—CH₂OCH₃). In some embodiments, R⁴ is hydroxymethyl (—CH₂OH). In some embodiments, R⁴ is aminomethyl (e.g., —CH₂NH₂, —CH₂NHCH₃, —CH₂N(CH₃)₂.

In some embodiments, R⁴ is —C₁-C₆ haloalkyl. In some embodiments, R⁴ is trifluoromethyl (—CF₃). In other embodiments, R⁴ is difluoromethyl (—CHF₂).

In some embodiments, R⁴ is C₃-C₉ cycloalkyl (e.g., cyclopropyl, cyclobutyl, cyclopentyl, cyclohexyl). In some embodiments, R⁴ is cyclopropyl. In some embodiments R⁴ is cyclobutyl. In some embodiments, R⁴ is cyclopentyl. In some embodiments, R⁴ is cyclohexyl.

In some embodiments, R⁴ is 3-10 membered heterocyclyl. In some embodiments, R⁴ is 3-6 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁴ is oxetanyl. In some embodiments, R⁴ is tetrahydropyranyl. In some embodiments, R⁴ is tetrahydrofuranyl. In some embodiments, R⁴ is azetidinyl. In some embodiments, R⁴ is pyrrolidinyl. In some embodiments, R⁴ is piperidinyl. In some embodiments, R⁴ is piperazinyl. In some embodiments, R⁴ is morpholinyl. In some embodiments, R⁴ is azepanyl.

In some embodiments R⁴ is cycloalkylalkyl (e.g., cyclopropylmethyl, cyclobutylmethyl, cyclopentylmethyl, cyclohexylmethyl, cycloheptylmethyl).

In some embodiments, R⁴ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R⁴ is arylalkyl. In some embodiments, R⁴ is benzyl.

In some embodiments, R⁴ is heteroarylalkyl (e.g., pyridinylmethyl, thiazolylmethyl, triazolylmethyl, pyrazolylmethyl).

In some embodiments, R⁴ is —OR^{a4} wherein R^{a4} is as defined in any of the embodiments described herein (e.g., hydroxy (—OH), methoxy, ethoxy, propoxy, isopropoxy, cyclopropyloxy, cyclobutyloxy). In some embodiments, R⁴ is hydroxy. In some embodiments, R⁴ is methoxy. In some embodiments, R⁴ is ethoxy. In some embodiments, R⁴ is propoxy. In some embodiments, R⁴ is isopropoxy. In some embodiments, R⁴ is —C₁-C₆ haloalkoxy. In some embodiments, R⁴ is trifluoromethoxy (—OCF₃), In other embodiments, R⁴ is difluoromethoxy (—OCHF₂).

In some embodiments, R⁴ is —N(R^{a4})₂ wherein R^{a4} is as defined in any of the embodiments described herein (e.g., —NH₂, —NHR^{a4}, —N(CH₃)R^{a4}). In some embodiments, R⁴ is —NH₂. In some embodiments, R⁴ is —NHR^{a4} (e.g., —NHCH₃, —NHCH₂CH₃, —NHPr, —NHⁱPr, —NHcyclopropyl, —NHcyclobutyl). In some embodiments, R⁴ is —N(CH₃)R^{a4} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R⁴ is —C(═O)R^{a4} or —C(═O)OR^{a4} wherein R^{a4} is as defined in any of the embodiments described herein. In some embodiments, R⁴ is —C(═O)R^{a4} wherein R^{a4} is as defined in any of the embodiments described herein. In some embodiments, R⁴ is —C(═O)alkyl. In some embodiments, R⁴ is —C(═O)CH₃, —C(═O)cyclopropyl, —C(═O)cyclobutyl, —C(═O)^{t}Bu, —C(═O)ⁱPr, —C(═O)CH₂CH₂CH₃ or —C(═O)OCH₃. In some embodiments, R⁴ is acetyl (—C(═O)CH₃). In some embodiments, R⁴ is —C(═O)OR^{a4}. In some embodiments, R⁴ is —COOH. In some embodiments, R⁴ is COOCH₃.

In some embodiments, R⁴ is —NR^{a4}C(═O)R^{a4} wherein R^{a4} is as defined in any of the embodiments described herein. In certain embodiments, R⁴ is —NHC(═O)R^{a4} (e.g., —NHC(═O)CH₃, —NHC(═O)CH₂CH₃, —NHC(═O)CH₂CH₂CH₃, —NHC(═O)ⁱPr, —NHC(═O)Bu, —NHC(═O)Bu, —NHC(═O)Cyclopropyl, —NHC(═O)Cyclobutyl). In some embodiments, R⁴ is —N(CH₃)C(═O)R^{a4} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R⁴ is —NR^{a4}C(═O)OR^{a4} wherein R^{a4} is as defined in any of the embodiments described herein. In certain embodiments, R⁴ is —NHC(═O)OR^{a4} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R⁴ is —N(CH₃)C(═O)OR^{a4} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R⁴ is —C(═O)N(R^{a4})₂ wherein R^{a4} is as defined in any of the embodiments described herein (e.g., —C(═O)NH₂, —C(═O)NHR^{a4}, —C(═O)N(CH₃)R^{a4}). In some embodiments, R⁴ is —C(═O)NH₂. In certain embodiments, R⁴ is —C(═O)NHR^{a4} (e.g., —C(═O)NHCH₃, —C(═O)NHCH₂CH₃, —C(═O)NHPr, —C(═O)NHPr, —C(═O)NHBu, —C(═O)NH^{t}Bu, —C(═O)NHCyclopropyl, —C(═O)NHCyclobutyl). In certain embodiments, R⁴ is —C(═O)N(CH₃)R^{a4} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)Bu, —C(═O)N(CH₃)Cyclopropyl, —C(═O)N(CH₃)Cyclobutyl).

In some embodiments, R⁴ is —C(═O)N(OR^{a4})(R^{a4}) wherein R^{a4} is as defined in any of the embodiments described herein. In certain embodiments, R⁴ is —C(═O)NH(OR^{a4}) (e.g., —C(═O)NHOH, —C(═O)NHOCH₃). In some embodiments, R⁴ is —C(═O)NHOH.

In some embodiments, R⁴ is —OC(═O)N(R^{a4})₂ wherein R^{a4} is as defined in any of the embodiments described herein. In certain embodiments, R⁴ is —OC(═O)NHR^{a4} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁴ is —OC(═O)N(CH₃)R^{a4} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁴ is —S(═O)R^{a4} wherein R^{a4} is as defined in any of the embodiments described herein. In certain embodiments, R⁴ is —S(═O)alkyl (e.g., —S(═O)CH₃, —S(═O)CH₂CH₃, —S(═O)CH₂CH₂CH₃, —S(═O)ⁱPr). In certain embodiments, R⁴ is —S(═O)cycloalkyl (e.g., —S(═O)cyclopropyl, —S(═O)cyclobutyl, —S(═O)cyclopentyl, —S(═O)cyclohexyl).

In some embodiments, R⁴ is —S(═O)₂R^{a4} wherein R^{a4} is as defined in any of the embodiments described herein. In certain embodiments, R⁴ is —S(═O)₂alkyl (e.g., —S(═O)₂CH₃, —S(═O)₂CH₂CH₃, —S(═O)₂Pr, —S(═O)₂ⁱPr). In certain embodiments, R⁴ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁴ is S(═O)₂aryl (e.g., —S(═O)₂phenyl).

In some embodiments, R⁴ is —SR^{a4} wherein R^{a4} is as defined in any of the embodiments described herein. In certain embodiments, R⁴ is -Salkyl (e.g., —SCH₃, —SCH₂CH₃, —SPr, —SⁱPr). In certain embodiments, R⁴ is -Scycloalkyl (e.g., -Scyclopropyl, —Scyclobutyl, —Scyclopentyl, —Scyclohexyl). In certain embodiments, R⁴ is -Saryl (e.g., -Sphenyl).

In some embodiments, R⁴ is —S(═O)(═NR^{a4})R^{a4} wherein R^{a4} is as defined in any of the embodiments described herein. In certain embodiments, R⁴ is —S(═O)(═NH)R^{a4} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)Bu, —S(═O)(═NH)Bu, —S(═O)(═NH). Cyclopropyl, —S(═O)(═NH)Cyclobutyl). In some embodiments, R⁴ is —S(═O)(═NCH₃)R^{a4} (e.g., —S(═O)(═NCH₃)CH₃, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁴ is —NR^{a4}S(═O)₂R^{a4} wherein R^{a4} is as defined in any of the embodiments described herein. In certain embodiments, R⁴ is —NHS(═O)₂alkyl (e.g., —NHS(═O)₂CH₃, —NHS(═O)₂CH₂CH₃, —NHS(═O)₂Pr, —NHS(═O)₂ⁱPr). In certain embodiments, R⁴ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁴ is —N(CH₃) S(═O)₂alkyl (e.g., —N(CH₃) S(═O)₂CH₃, —N(CH₃) S(═O)₂CH₂CH₃, —N(CH₃) S(═O)₂Pr, —N(CH₃) S(═O)₂ⁱPr). In certain embodiments, R⁴ is —N(CH₃) S(═O)₂cycloalkyl (e.g., —N(CH₃) S(═O)₂cyclopropyl, —N(CH₃) S(═O)₂cyclobutyl, —N(CH₃) S(═O)₂cyclopentyl, —N(CH₃) S(═O)₂cyclohexyl).

In some embodiments, R⁴ is —S(═O)₂N(R^{a4})₂ wherein R^{a4} is as defined in any of the embodiments described herein. (e.g., —S(═O)₂NH₂, —S(═O)₂NHR^{a4}, —S(═O)₂N(CH₃)R^{a4}). In some embodiments, R⁴ is —S(═O)₂NH₂. In some embodiments, R⁴ is —S(═O)₂NHR^{a4} (e.g., —S(═O)₂NHCH₃, —S(═O)₂NHCH₂CH₃, —S(═O)₂NHPr, —S(═O)₂NHⁱPr, —S(═O)₂NHcyclopropyl, —S(═O)₂NHcyclobutyl). In some embodiments, R⁴ is —S(═O)₂N(CH₃)R^{a4} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, Ring B is selected from the group consisting of —C₃-C₁₀ carbocyclyl, 3-10 membered heterocyclyl, C₆-C₁₀ aryl and 5-10 membered heteroaryl wherein each carbocyclyl, heterocyclyl, aryl and heteroaryl is optionally substituted at any available position.

In some embodiments, each carbocyclyl, heterocyclyl, aryl and heteroaryl of Ring B is substituted at any available position with 0, 1, 2 or 3 instances of R³, wherein each R³ is as defined in any of the embodiments described herein.

In some embodiments, Ring B is independently selected from the group consisting of a —C₃-C₁₀ monocyclic or bicyclic carbocyclyl (e.g., a —C₃-C₁₀ monocyclic or bicyclic cycloalkyl, a partially unsaturated-C₃-C₁₀ monocyclic or bicyclic carbocyclyl, a —C₃-C₇ monocyclic carbocyclyl fused with phenyl or with a 5-6-membered heterocyclyl or heteroaryl ring containing 1-3 atoms independently selected from the group consisting of N, O, S or oxidized forms thereof), 3-10 membered mono or bicyclic heterocyclyl (e.g., a 3-8 membered monocyclic heterocyclyl containing 1-3 heteroatoms selected from the group consisting of N, O and S or oxidized forms thereof, a 4-10 membered bicyclic heterocyclyl containing 1-3 heteroatoms independently selected from the group consisting of N, O and S or oxidized forms thereof), —C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof), 5-6 membered monocyclic heteroaryl (e.g., containing 1-4 heteroatoms independently selected from the group consisting of N, O and S), 8-10 membered bicyclic heteroaryl (e.g., containing 1-4 heteroatoms independently selected from the group consisting of N, O and S) wherein each carbocyclyl, heterocyclyl, aryl and heteroaryl is optionally substituted (e.g., substituted with 0, 1, 2 or 3 instances of R³ wherein R³ is as defined in any of the embodiments described herein).

In some embodiments, Ring B is selected from the group consisting of —C₃-C₁₀ monocyclic or bicyclic carbocyclyl (e.g., cyclopropyl, cyclobutyl, cyclopentyl, cyclohexyl, cycloheptyl, bicyclo[1.1.1]pentan-yl, 4,5,6,7-tetrahydro-1H-indazolyl, spiro[3.3]heptanyl), 3-10 membered mono or bicyclic heterocyclyl (e.g., oxetanyl, azepanyl, piperidinyl, pyrrolidinyl, tetrahydrofuranyl, tetrahydropyranyl, 1,2-dihydropyridinyl, morpholinyl), C₆-C₁₀ mono or bicyclic aryl (e.g., phenyl, naphthyl, 1,2,3,4-tetrahydronaphthalenyl, 2,3-dihydro-1H-indenyl, 1,2,3,4 tetrahydroquinolinyl, 1,2 dihydroquinolinyl, 1,2-dihydroisoquinolinyl, 1,2,3,4 tetrahydroisoquinolinyl, chromanyl, indolinyl, isoindolinyl, 3,4-dihydro-2H-benzo[b][1,4]oxazinyl, 2,3-dihydrobenzofuranyl, benzo[d][1,3]dioxolyl, 2,3-dihydro-1H-benzo[d]imidazolyl), 5-6 membered monocyclic heteroaryl (e.g., thiophenyl, thiazolyl, pyrazolyl, imidazolyl, oxazolyl, pyridinyl, pyrimidinyl), 8-10 membered bicyclic heteroaryl (e.g., benzo[d]isothiazolyl, indolyl, benzofuranyl, 1H-indazolyl, 2-H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, isoquinolinyl, benzo[d]imidazolyl, benzo[d]thiazolyl, benzo[d]oxazolyl, [1,2,4]triazolo[4,3-a]pyridinyl, imidazo[1,2-a]pyridinyl, imidazo[1,5-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl) wherein each cycloalkyl, heterocyclyl, aryl and heteroaryl is optionally substituted (e.g., substituted with 0, 1, 2 or 3 instances of R³ wherein R³ is as defined in any of the embodiments described herein).

In certain embodiments, Ring B is selected from the group consisting of C₆-C₁₀ aryl and 5-10 membered heteroaryl, wherein the aryl and heteroaryl are optionally substituted (e.g., substituted at available positions with 0, 1, 2 or 3 instances of R³, wherein each R³ is as defined in any of the embodiments described herein).

In some embodiments, Ring B is selected from the group consisting of phenyl, naphthalenyl, C₃-C₇ monocyclic cycloalkyl, 3-7 membered monocyclic heterocyclyl, 5-6 membered monocyclic heteroaryl, and 8-10 membered bicyclic heteroaryl, each optionally substituted at any available position.

In some embodiments, Ring B is selected from the group consisting of C₆-C₁₀ aryl and 8-10 membered bicyclic heteroaryl wherein the aryl and heteroaryl are optionally substituted (e.g., substituted at available positions with 0, 1, 2 or 3 instances of R³, wherein each R³ is as defined in any of the embodiments described herein).

In some embodiments, Ring B is selected from the group consisting of:

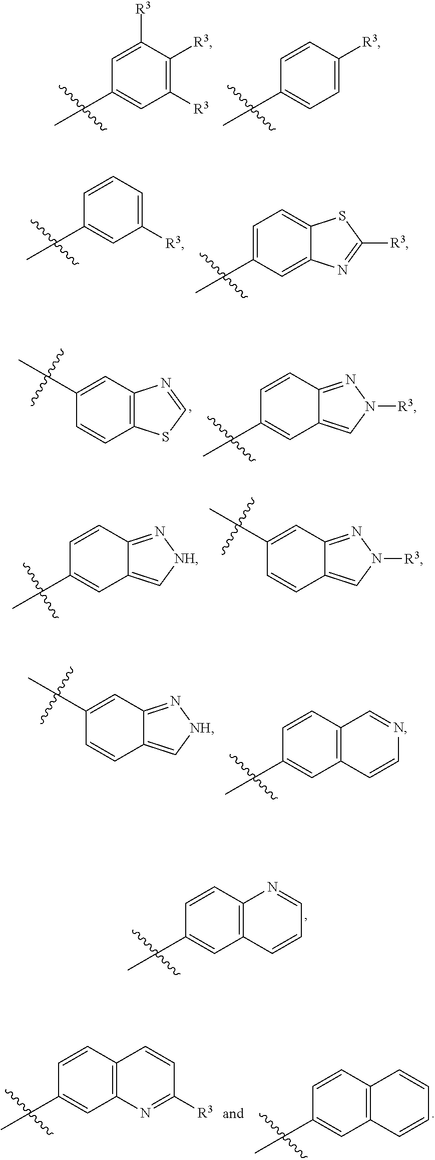

In some embodiments, Ring B is selected from the group consisting of:

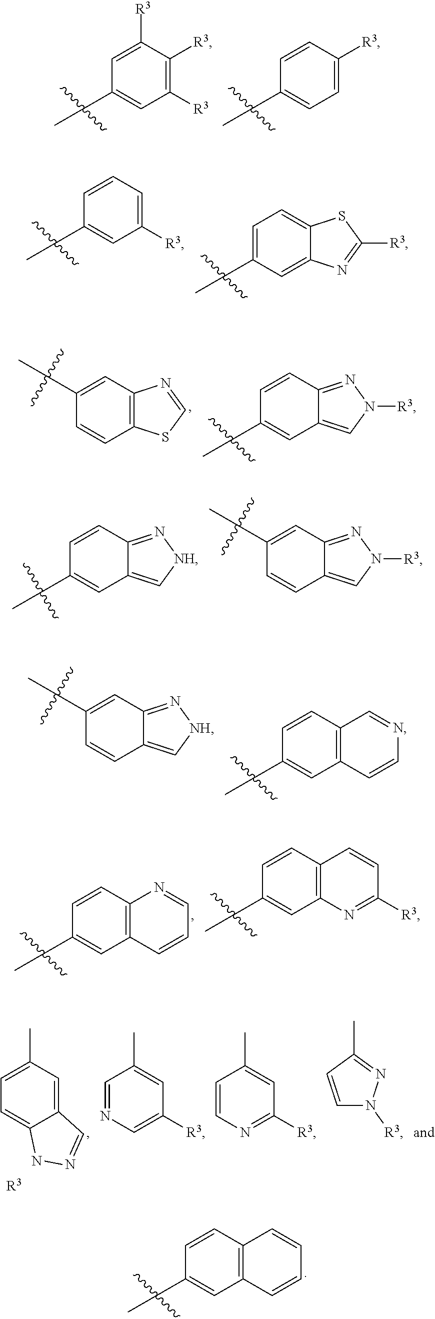

In some embodiments, Ring B is selected from the group consisting of:

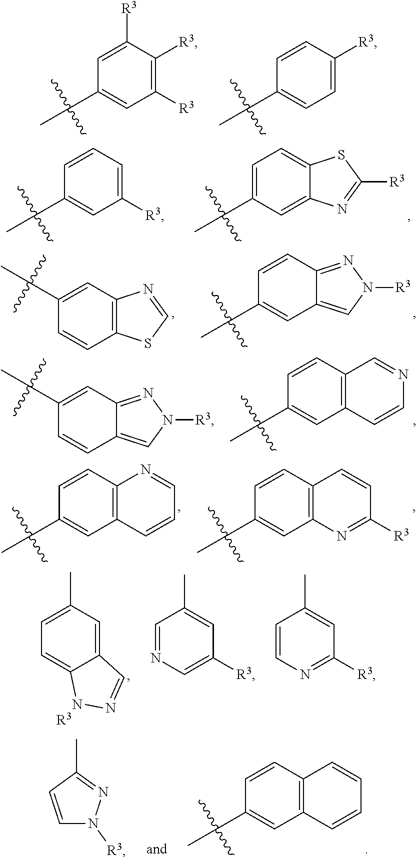

In some embodiments, Ring B is selected from the group consisting of:

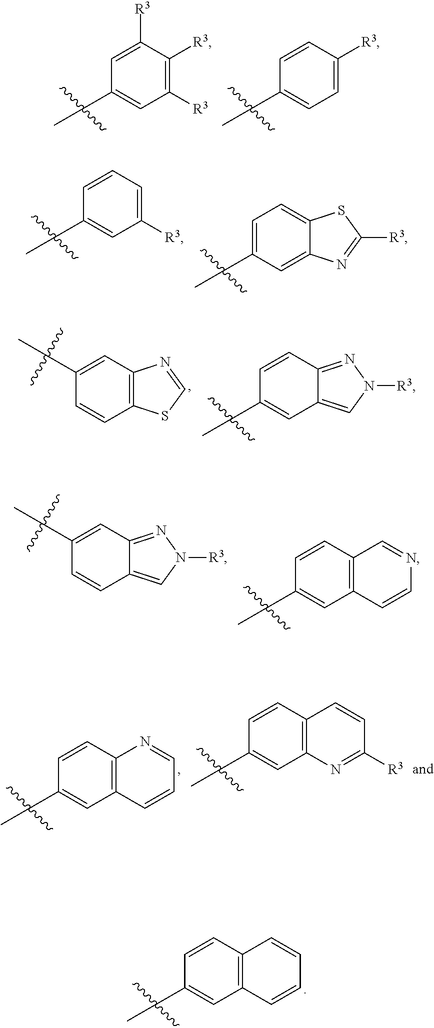

In some embodiments, Ring B is selected from the group consisting of:

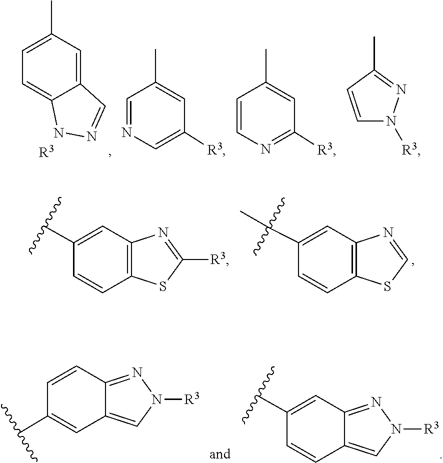

In some embodiments, Ring B is selected from the group consisting of:

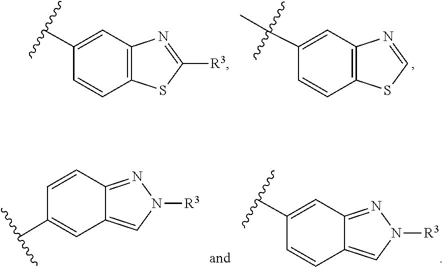

In some embodiments, Ring B is selected from the group consisting of:

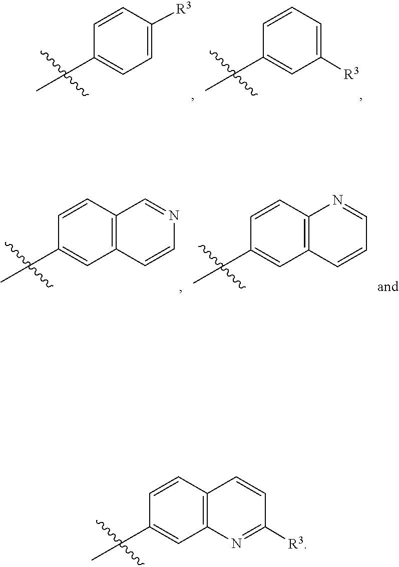

In some embodiments, Ring B is selected from the group consisting of:

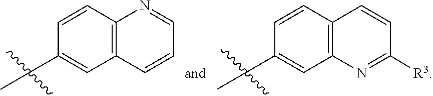

In some embodiments, Ring B is selected from the group consisting of:

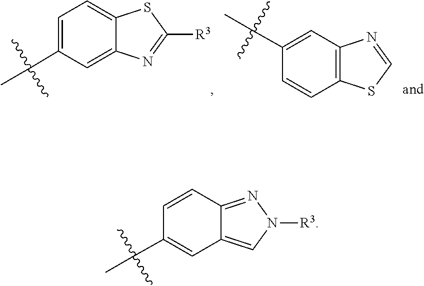

In some embodiments, Ring B is selected from the group consisting of cyclopropyl, cyclobutyl, cyclopentyl, cyclohexyl, cycloheptyl, oxetanyl, tetrahydrofuranyl, tetrahydropyranyl, azetidinyl, pyrrolidinyl, piperidinyl, piperazinyl, thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, isoquinolinyl, 1,2,3,4-tetrahydroquinolinyl, 1,2,3,4-tetrahydroisoquinolinyl, 2,3-dihydrobenzofuranyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted at any available position (e.g., substituted with 0, 1, 2 or 3 instances of R³, wherein R³ is as defined in any of the embodiments described herein).

In some embodiments, Ring B is selected from the group consisting of cyclopropyl, cyclobutyl, cyclopentyl, cyclohexyl, cycloheptyl, oxetanyl, tetrahydrofuranyl, tetrahydropyranyl, azetidinyl, pyrrolidinyl, piperidinyl, piperazinyl, thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted (e.g., substituted at available positions with 0, 1, 2 or 3 instances of R³, wherein each R³ is as defined in any of the embodiments described herein).

In some embodiments, Ring B is selected from the group consisting of cyclopropyl, cyclobutyl, cyclopentyl, cyclohexyl, tetrahydrofuranyl, tetrahydropyranyl, thiophenyl, oxazolyl, pyrazolyl, phenyl, naphthalenyl, pyridinyl, 1H-indazolyl, 2H-indazolyl, quinolinyl, isoquinolinyl, 1,2,3,4-tetrahydroquinolinyl, 1,2,3,4-tetrahydroisoquinolinyl, 2,3-dihydrobenzofuranyl and benzo[d]thiazolyl, each optionally substituted at any available position (e.g., substituted with 0, 1, 2 or 3 instances of R³, wherein R³ is as defined in any of the embodiments described herein).

In some embodiments, Ring B is selected from the group consisting of cyclobutyl, cyclopentyl, tetrahydropyranyl, pyrazolyl, thiophenyl, oxazolyl, pyridinyl, phenyl, naphthalenyl, 1H-indazolyl, 2H-indazolyl, quinolinyl, isoquinolinyl and benzo[d]thiazolyl, each optionally substituted (e.g., substituted at available positions with 0, 1, 2 or 3 instances of R³, wherein each R³ is as defined in any of the embodiments described herein).

In some embodiments, Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted at any available position (e.g., substituted with 0, 1, 2 or 3 instances of R³, wherein R³ is as defined in any of the embodiments described herein).

In some embodiments, Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-3-yl, tetrahydropyran-4-yl, pyrazol-5-yl, pyrazol-3-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted (e.g., substituted at available positions with 0, 1, 2 or 3 instances of R³, wherein each R³ is as defined in any of the embodiments described herein).

In some embodiments, Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-3-yl, tetrahydropyran-4-yl, pyrazol-5-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted (e.g., substituted at available positions with 0, 1, 2 or 3 instances of R³, wherein each R³ is as defined in any of the embodiments described herein).

In yet some embodiments, Ring B is selected from the group consisting of cyclopentyl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H-indazol-5-yl, 2H-indazol-6-yl, 2H-indazol-5-yl, quinolin-6-yl, quinolin-7-yl, isoquinolin-6-yl, benzo[d]thiazol-5-yl, each optionally substituted (e.g., substituted at available positions with 0, 1, 2 or 3 instances of R³, wherein each R³ is as defined in any of the embodiments described herein).

In yet some embodiments, Ring B is selected from the group consisting of cyclopentyl, tetrahydropyran-3-yl, tetrahydropyran-4-yl, pyrazol-5-yl, thiophen-3-yl, oxazol-5-yl, pyridin-3-yl, pyridin-4-yl, phenyl, naphthalen-2-yl, 2H-indazol-6-yl, 2H-indazol-5-yl, quinolin-6-yl, quinolin-7-yl, isoquinolin-6-yl, benzo[d]thiazol-5-yl, each optionally substituted (e.g., substituted at available positions with 0, 1, 2 or 3 instances of R³, wherein each R³ is as defined in any of the embodiments described herein).

In some embodiments, Ring B is selected from the group consisting of cyclopropyl, cyclobutyl, cyclopentyl, cyclohexyl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H indazol-5-yl, 1H indazol-4-yl, 2H-indazol-6-yl, 2H-indazol-5-yl, quinolin-6-yl, quinolin-7-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl and benzo[d]thiazol-5-yl, each optionally substituted at any available position (e.g., substituted with 0, 1, 2 or 3 instances of R³, wherein R³ is as defined in any of the embodiments described herein).

In some embodiments, Ring B is unsubstituted. In some embodiments, Ring B is substituted with 1 instance of R³. In some embodiments, Ring B is substituted with 2 instances of R³. In some embodiments, Ring B is substituted with 3 instances of R³.

In some embodiments, Ring B is —C₃-C₁₀ carbocyclyl. In some embodiments, the carbocyclyl is not further substituted. In some embodiments, the carbocyclyl is substituted with 0, 1, 2 or 3 instances of R³, wherein R³ is as described herein. In some embodiments, the carbocyclyl is substituted with 1 or 2 instances of halo (e.g., —F, —C₁), -Me, -Et, -iPr, —OH, ═O or —CN. In some embodiments, the carbocyclyl is a —C₃-C₁₀ mono or bicyclic cycloalkyl (e.g., a monocyclic cycloalkyl, a fused bicyclic cycloalkyl, a spiro cycloalkyl or a bridged cycloalkyl). In some embodiments, the carbocyclyl is a —C₃-C₇ monocyclic cycloalkyl (e.g., cyclopropyl, cyclobutyl, cyclopentyl, cyclohexyl). In some embodiments, Ring Bis cyclopropyl. In some embodiments Ring B is cyclobutyl. In some embodiments, Ring B is cyclopentyl. In some embodiments, Ring B is cyclohexyl. In some embodiments, the carbocyclyl is a —C₄-C₁₀ bicyclic cycloalkyl. In some embodiments, Ring B is a —C₄-C₁₀ fused cycloalkyl (e.g., decahydronaphthyl, octahydroindenyl). In some embodiments, Ring B is a —C₄-C₁₀ spiro cycloalkyl (e.g., spiro[4.5]decanyl, spiro[4.4]nonanyl, spiro[3.3]heptanyl, spiro[3.4]octanyl).

In some embodiments, the carbocyclyl is a —C₃-C₇ monocyclic carbocyclyl fused with phenyl (e.g., 1,2,3,4-tetrahydronaphthalenyl, 2,3-dihydro-1H-indenyl wherein the attachment point is on the saturated ring). In some embodiments, Ring B is a —C₃-C₇ monocyclic carbocyclyl fused with a 5-6-membered heterocyclyl ring containing 1-3 atoms independently selected from the group consisting of N, O, S or oxidized forms thereof (e.g., octahydrochromenyl, decahydroisoquinolinyl, decahydroquinolinyl, octahydroindolyl, octahydroisoindolyl, hexahydro chromane, hexahydro isoquinoline, hexahydroquinoline, octahydrobenzofuran wherein the attachment point is on the carbocyclic ring). In some embodiments, Ring B is a —C₃-C₇ monocyclic carbocyclyl fused with a 5-6-membered heteroaryl ring containing 1-3 atoms independently selected from the group consisting of N, O, S or oxidized forms thereof (e.g., 4,5,6,7-tetrahydro-1H-indazolyl).

In some embodiments, Ring B is C₃-C₇ monocyclic cycloalkyl, optionally substituted at any available position (e.g., substituted with 0, 1, 2 or 3 instances of R³, wherein R³ is as defined in any of the embodiments described herein).

In some embodiments, Ring B is selected from the group consisting of cyclopropyl, cyclobutyl, cyclopentyl and cyclohexyl, each optionally substituted at any available position (e.g., substituted with 0, 1, 2 or 3 instances of R³, wherein R³ is as defined in any of the embodiments described herein).

In some embodiments, Ring B is cyclopropyl, optionally substituted at any available position (e.g., substituted with 0, 1, 2 or 3 instances of R³, wherein R³ is as defined in any of the embodiments described herein).

In some embodiments, Ring B is cyclobutyl, optionally substituted at any available position (e.g., substituted with 0, 1, 2 or 3 instances of R³, wherein R³ is as defined in any of the embodiments described herein).

In some embodiments, Ring B is cyclopentyl, optionally substituted at any available position (e.g., substituted with 0, 1, 2 or 3 instances of R³, wherein R³ is as defined in any of the embodiments described herein).

In some embodiments, Ring B is cyclohexyl, optionally substituted at any available position (e.g., substituted with 0, 1, 2 or 3 instances of R³, wherein R³ is as defined in any of the embodiments described herein).

In some embodiments, Ring B is selected from the group consisting of:

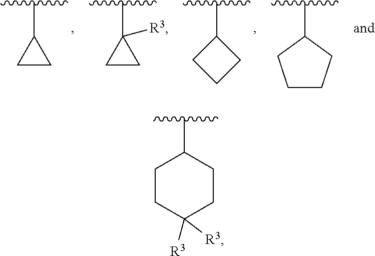

wherein each R³ is independently as defined in any of the embodiments described herein.

In some embodiments, Ring B is

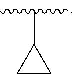 [SMILES: *C1CC1]

In some embodiments, Ring B is

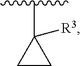

wherein R³ is as defined in any of the embodiments described herein.

In some embodiments, Ring B is

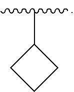

wherein R³ is as defined in any of the embodiments described herein. In some embodiments, Ring B is

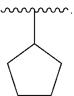 [SMILES: *C1CCCC1]

In some embodiments, Ring B is

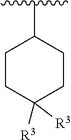

wherein R³ is as defined in any of the embodiments described herein.

In some embodiments, Ring B is a 3-10 membered mono or bicyclic heterocyclyl. In some embodiments, Ring B is a 3-8 membered monocyclic heterocyclyl containing 1-3 heteroatoms selected from the group consisting of N, O and S or oxidized forms thereof (e.g., oxetanyl, tetrahydropyranyl, tetrahydrofuranyl, azetidinyl, pyrrolidinyl, piperidinyl, piperazinyl, morpholinyl, azepanyl, tetrahydropyridinyl). In some embodiments, ring B is oxetanyl (e.g., oxetan-3-yl). In some embodiments, Ring B is selected from the group consisting of tetrahydrofuranyl and tetrahydropyranyl, each optionally substituted at any available position (e.g., substituted with 0, 1, 2 or 3 instances of R³, wherein R³ is as defined in any of the embodiments described herein).

In some embodiments, Ring B is selected from the group consisting of tetrahydrofuran-3-yl, tetrahydropyran-2-yl, tetrahydropyran-3-yl and tetrahydropyran-4-yl, each optionally substituted at any available position (e.g., substituted with 0, 1, 2 or 3 instances of R³, wherein R³ is as defined in any of the embodiments described herein).

In some embodiments, ring B is tetrahydrofuranyl (e.g., tetrahydrofuran-2-yl, tetrahydrofuran-3-yl). In some embodiments, Ring B is tetrahydrofuran-3-yl, optionally substituted at any available position (e.g., substituted with 0, 1, 2 or 3 instances of R³, wherein R³ is as defined in any of the embodiments described herein).

In some embodiments, ring B is tetrahydropyranyl (e.g., tetrahydropyran-2-yl, tetrahydropyran-3-yl, tetrahydropyran-4-yl). In some embodiments, Ring B is tetrahydropyran-2-yl, optionally substituted at any available position (e.g., substituted with 0, 1, 2 or 3 instances of R³, wherein R³ is as defined in any of the embodiments described herein).

In some embodiments, Ring B is tetrahydropyran-3-yl, optionally substituted at any available position (e.g., substituted with 0, 1, 2 or 3 instances of R³, wherein R³ is as defined in any of the embodiments described herein).

In some embodiments, Ring B is tetrahydropyran-4-yl, optionally substituted at any available position (e.g., substituted with 0, 1, 2 or 3 instances of R³, wherein R³ is as defined in any of the embodiments described herein).

In some embodiments, Ring B is selected from the group consisting of:

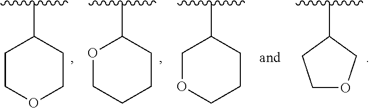

In some embodiments, Ring B is

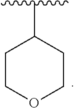 [SMILES: *C1CCOCC1]

In some embodiments, Ring B is

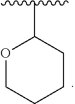 [SMILES: *C1CCCCO1]

In some embodiments, Ring B is

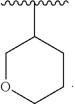 [SMILES: *C1CCCOC1]

In some embodiments, Ring B is

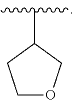 [SMILES: *C1CCOC1]

In some embodiments, ring B is azetidinyl (e.g., azetidin-3-yl). In some embodiments, ring B is pyrrolidinyl (e.g., pyrrolidin-2-yl, pyrrolidin-3-yl). In some embodiments, ring B is piperidinyl (e.g., piperidin-2-yl, piperidin-3-yl, piperidin-4-yl). In some embodiments, ring B is piperazinyl (e.g., piperazin-4-yl). In some embodiments, ring B is morpholinyl. In some embodiments, ring B is tetrahydropyridinyl. In some embodiments, Ring B is azepanyl.

In some embodiments, Ring B is a 4-10 membered bicyclic heterocyclyl containing 1-3 heteroatoms independently selected from the group consisting of N, O and S or oxidized forms thereof. In some embodiments, the mono or bicyclic heterocyclyl is not further substituted. In some embodiments, the heterocyclyl is substituted with 1 or 2 instances of halo (e.g., —F, —C₁), -Me, -Et, -iPr, —OH, ═O or —CN. In some instances, the heterocyclyl is substituted with ═O (e.g., 2-oxo-1,2-dihydropyridin-4-yl).

In some embodiments, Ring B is an optionally substituted 6-10 membered mono or bicyclic aryl (i.e., a C₆-C₁₀ aryl, e.g., naphthalenyl, phenyl, 1,2,3,4-tetrahydroquinolinyl, 1,2,3,4-tetrahydroisoquinolinyl, 2,3-dihydrobenzofuranyl). In some embodiments, Ring B is substituted with 0, 1, 2 or 3 instances of R³, wherein R³ is as described herein. In some embodiments, Ring B is selected from the group consisting of naphthalenyl, phenyl, 1,2,3,4-tetrahydroquinolinyl, 1,2,3,4-tetrahydroisoquinolinyl and 2,3-dihydrobenzofuranyl, each optionally substituted at any available position (e.g., substituted with 0, 1, 2 or 3 instances of R³, wherein R³ is as defined in any of the embodiments described herein).

In some embodiments, Ring B is selected from the group consisting of naphthalenyl, phenyl, 1,2,3,4-tetrahydroquinolinyl, 1,2,3,4-tetrahydroisoquinolinyl and 2,3-dihydrobenzofuranyl, each optionally substituted at any available position (e.g., substituted with 0, 1, 2 or 3 instances of R³, wherein R³ is as defined in any of the embodiments described herein).

In some embodiments, Ring B is selected from the group consisting of phenyl, naphthalen-2-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each optionally substituted at any available position (e.g., substituted with 0, 1, 2 or 3 instances of R³, wherein R³ is as defined in any of the embodiments described herein).

In some embodiments, Ring B is naphthalenyl or phenyl, each optionally substituted at any available position (e.g., substituted with 0, 1, 2 or 3 instances of R³, wherein R³ is as defined in any of the embodiments described herein). In some embodiments, Ring B is optionally substituted phenyl. In some embodiments, Ring B is phenyl substituted with 0, 1, 2 or 3 instances of R³, wherein each R³ is independently as described herein. In some embodiments, the phenyl is unsubstituted. In some embodiments, the phenyl is substituted with one instance of R³. In some embodiments, the phenyl is substituted with 1 instance of R³ at the position para- to the attachment point to the piperidine. In some embodiments, the phenyl is substituted with 1 instance of R³ at the position meta- to the attachment point to the piperidine. In some embodiments, the phenyl is substituted with 2 instances of R³. In some embodiments, the phenyl is substituted with 3 instances of R³.

In some embodiments, Ring B is selected from the group consisting of:

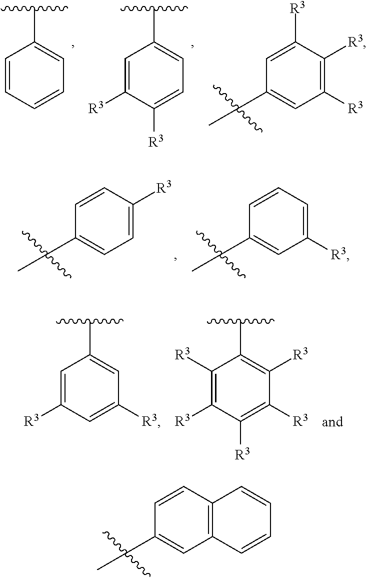

wherein each R³ is independently as defined in any of the embodiments described herein.

In some embodiments, Ring B is selected from the group consisting of:

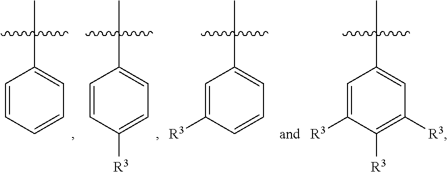

wherein each R³ is as defined in any of the embodiments described herein.

In some embodiments, Ring B is selected from the group consisting of:

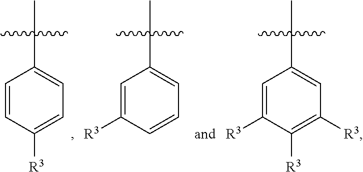

wherein each R³ is as defined in any of the embodiments described herein.

In some embodiments, Ring B is:

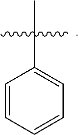 [SMILES: *c1ccccc1]

In some embodiments, Ring B is

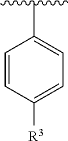

wherein each R³ is as defined in any of the embodiments described herein. In some embodiments, Ring B is:

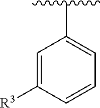

wherein each R³ is as defined in any of the embodiments described herein. In some embodiments, Ring B is

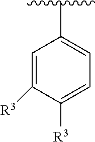

wherein R³ is as defined in any of the embodiments described herein. In some embodiments, Ring B is:

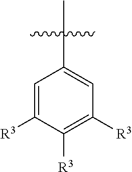

wherein each R³ is as defined in any of the embodiments described herein. In some embodiments, Ring B is

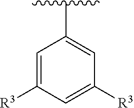

wherein R³ is as defined in any of the embodiments described herein. In some embodiments, Ring B is

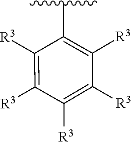

wherein R³ is as defined in any of the embodiments described herein. In some embodiments, Ring B is

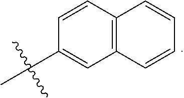 [SMILES: *c1ccc2ccccc2c1]

In some embodiments, the compounds of Formula (I) are of Formula (III1):

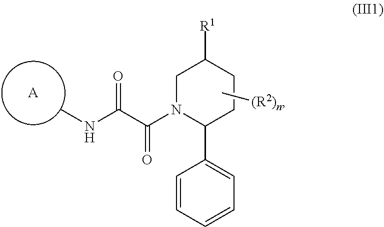

wherein Ring A, R¹, R² and n are as defined in any of the embodiments described herein and the phenyl is substituted with 0, 1, 2 or 3 instances of R³ as defined in any of the embodiments described herein. In some embodiments, the compounds of Formula (I) are of Formula (III1a):

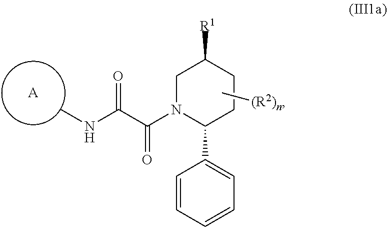

wherein Ring A, R¹, R² and n are as defined in any of the embodiments described herein and the phenyl is substituted with 0, 1, 2 or 3 instances of R³ as defined in any of the embodiments described herein. In yet some embodiments, compounds of Formula (I) are of Formula (III1b),

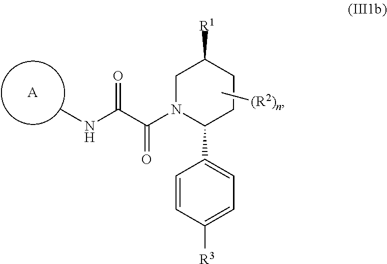

wherein Ring A, R¹, R² R³ and n are as defined in any of the embodiments described herein.

In other embodiments, compounds of Formula (I) are of Formula (III1c),

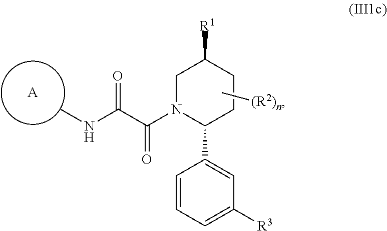

wherein Ring A, R¹, R² R³ and n are as defined in any of the embodiments described herein.

In certain embodiments, compounds of Formula (I) are of Formula (II11d),

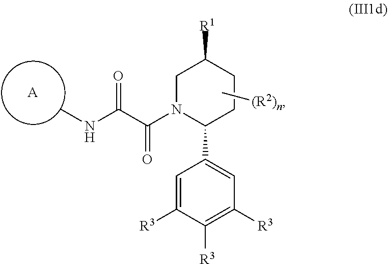

wherein Ring A, R¹, R² R³ and n are as defined in any of the embodiments described herein.

In some embodiments, Ring B is phenyl substituted with halo (e.g., fluoro, chloro, bromo), —CN or —CF₃. In some embodiments, Ring B is phenyl substituted with halo (e.g., fluoro, chloro, bromo). In some embodiments, Ring B is phenyl substituted with —CN. In some embodiments, Ring B is phenyl substituted with —CF₃.

In some embodiments, Ring B is an optionally substituted 9-10 membered bicyclic aryl (e.g., naphthalenyl). In some embodiments, Ring B is naphthalenyl (e.g., naphthalen-1-yl, naphthalen-2-yl). In some embodiments, Ring B is naphthalen-2-yl. In some embodiments, Ring B is an optionally substituted bicyclic aryl containing a phenyl ring fused with a C₅-C₆ carbocycle (e.g., tetrahydronaphthyl, dihydroindenyl). In some embodiments, Ring B is 1,2,3,4-tetrahydronaphthalenyl. In some embodiments, Ring B is 2,3-dihydro-1H-indenyl. In some embodiments, Ring B is an optionally substituted bicyclic aryl containing a phenyl ring fused with a 5-6 membered heterocycle containing 1-3 heteroatoms independently selected from the group consisting of N, O and S or oxidized forms thereof (e.g., 1,2,3,4 tetrahydroquinolinyl, 1,2 dihydroquinolinyl, 1,2-dihydroisoquinolinyl, tetrahydroisoquinolinyl, chromanyl, indolinyl, isoindolinyl, dihydrobenzoxazinyl, dihydrobenzofuranyl, benzodioxolyl, dihydrobenzimidazolyl). In some embodiments, Ring B is selected from the group consisting of 1,2,3,4-tetrahydroquinolinyl, 1,2,3,4-tetrahydroisoquinolinyl and 2,3-dihydrobenzofuranyl, each optionally substituted at any available position (e.g., substituted with 0, 1, 2 or 3 instances of R³, wherein R³ is as defined in any of the embodiments described herein).

In some embodiments, Ring B is selected from the group consisting of 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each optionally substituted at any available position (e.g., substituted with 0, 1, 2 or 3 instances of R³, wherein R³ is as defined in any of the embodiments described herein).

In some embodiments, Ring B is 1,2,3,4-tetrahydroquinolinyl, optionally substituted at any available position (e.g., substituted with 0, 1, 2 or 3 instances of R³, wherein R³ is as defined in any of the embodiments described herein).

In some embodiments, Ring B is 1,2,3,4-tetrahydroquinolin-6-yl, optionally substituted at any available position (e.g., substituted with 0, 1, 2 or 3 instances of R³, wherein R³ is as defined in any of the embodiments described herein).

In some embodiments, Ring B is 1,2,3,4-tetrahydroisoquinolinyl, optionally substituted at any available position (e.g., substituted with 0, 1, 2 or 3 instances of R³, wherein R³ is as defined in any of the embodiments described herein).

In some embodiments, Ring B is 1,2,3,4-tetrahydroisoquinolin-6-yl, optionally substituted at any available position (e.g., substituted with 0, 1, 2 or 3 instances of R³, wherein R³ is as defined in any of the embodiments described herein).

In some embodiments, Ring B is 1,2,3,4-tetrahydroisoquinolin-7-yl, optionally substituted at any available position (e.g., substituted with 0, 1, 2 or 3 instances of R³, wherein R³ is as defined in any of the embodiments described herein).

In some embodiments, Ring B is 2,3-dihydrobenzofuranyl, optionally substituted at any available position (e.g., substituted with 0, 1, 2 or 3 instances of R³, wherein R³ is as defined in any of the embodiments described herein).

In some embodiments, Ring B is 2,3-dihydrobenzofuran-4-yl, optionally substituted at any available position (e.g., substituted with 0, 1, 2 or 3 instances of R³, wherein R³ is as defined in any of the embodiments described herein).

In some embodiments, Ring B is 2,3-dihydrobenzofuran-5-yl, optionally substituted at any available position (e.g., substituted with 0, 1, 2 or 3 instances of R³, wherein R³ is as defined in any of the embodiments described herein).

In some embodiments, Ring B is 2,3-dihydrobenzofuran-7-yl, optionally substituted at any available position (e.g., substituted with 0, 1, 2 or 3 instances of R³, wherein R³ is as defined in any of the embodiments described herein).

In some embodiments, the bicyclic aryl is unsubstituted. In some embodiments, Ring B is unsubstituted naphthalenyl (e.g., naphthalen-1-yl, naphthalen-2-yl). In some embodiments, Ring B is unsubstituted naphthalen-2-yl. In some embodiments, the bicyclic aryl is substituted with 0, 1, 2 or 3 instances of R³, wherein each R³ is as described herein. In some embodiments, the bicyclic aryl is substituted with 1 instance of R³. In some embodiments, the bicyclic aryl is substituted with one instance of R³ wherein R³ is selected from the group consisting of halo (e.g., —F, —Cl, Br), -Me, ═O.

In some embodiments, Ring B is selected from the group consisting of:

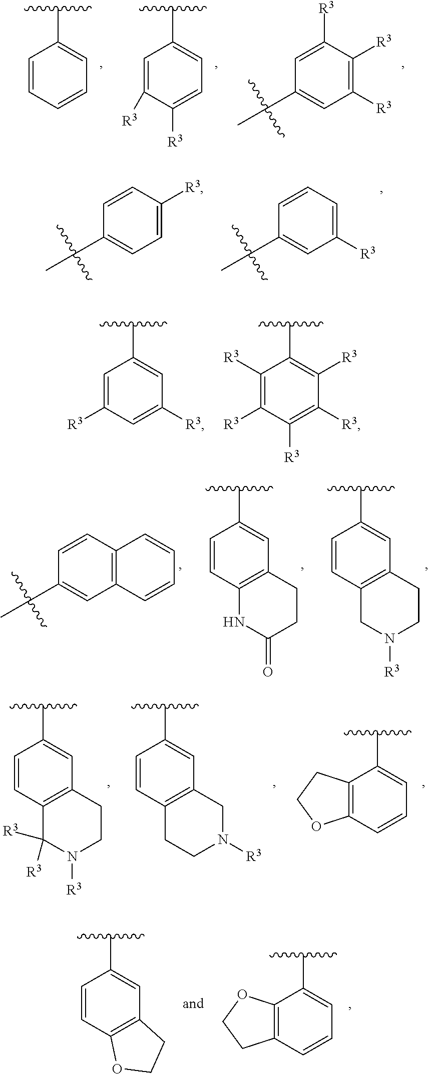

wherein each R³ is independently as defined in any of the embodiments described herein.

In some embodiments, Ring B is selected from the group consisting of:

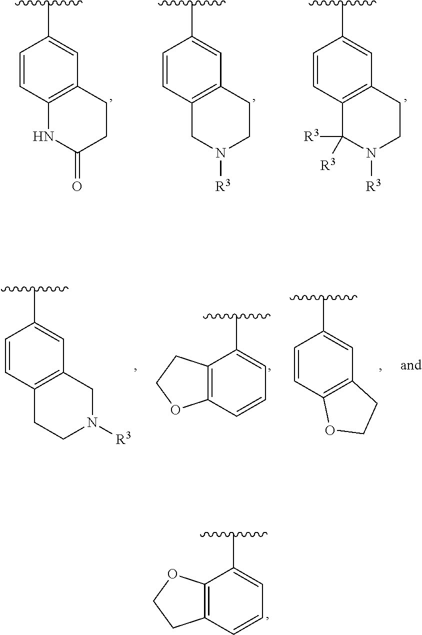

wherein each R³ is independently as defined in any of the embodiments described herein.

In some embodiments, Ring B is

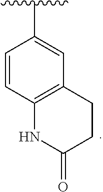 [SMILES: *c1ccc2c(c1)CCC(=O)N2]

In some embodiments, Ring B is

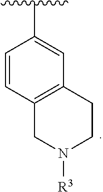

wherein R³ is as defined in any of the embodiments described herein. In some embodiments, Ring B is

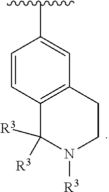

wherein R³ is as defined in any of the embodiments described herein. In some embodiments, Ring B is

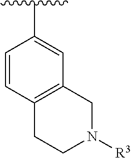

wherein R³ is as defined in any of the embodiments described herein. In some embodiments, Ring B is

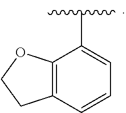 [SMILES: *c1cccc2c1OCC2]

In some embodiments, Ring B is

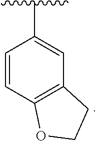 [SMILES: *c1ccc2c(c1)CCO2]

In some embodiments, Ring B is

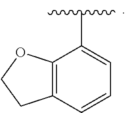 [SMILES: *c1cccc2c1OCC2]

In some embodiments, Ring B is an optionally substituted 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms).

In some embodiments, the 5-6 membered monocyclic heteroaryl is unsubstituted. In some embodiments, the 5-6 membered monocyclic heteroaryl is substituted with 0, 1, 2 or 3 instances of R³, wherein each R³ is as described herein. In some embodiments, the 5-6 membered monocyclic heteroaryl is substituted with 1 instance of R³. In some embodiments, the 5-6 membered monocyclic heteroaryl is substituted with 2 instances of R³. In some embodiments, the 5-6 membered monocyclic heteroaryl is substituted with 2 instances of R³. In some embodiments, the 5-6 membered monocyclic heteroaryl is substituted with 3 instances of R³.

In some embodiments, Ring B is selected from the group consisting of pyrazolyl, thiophenyl, oxazolyl and pyridinyl, each optionally substituted at any available position (e.g., substituted with 0, 1, 2 or 3 instances of R³, wherein R³ is as defined in any of the embodiments described herein).

In some embodiments, Ring B is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, Ring B is selected from the group consisting of pyrazolyl, thiophenyl and oxazolyl, each optionally substituted at any available position (e.g., substituted with 0, 1, 2 or 3 instances of R³, wherein R³ is as defined in any of the embodiments described herein).

In some embodiments, Ring B is pyrazolyl (e.g., pyrazol-3-yl, pyrazol-4-yl, pyrazol-5-yl). In some embodiments, Ring B is pyrrolyl (e.g., pyrrol-1-yl, pyrrol-2-yl, pyrrol-3-yl). In some embodiments, Ring B is thiophenyl (e.g., thiophen-2-yl, thiophen-3-yl). In some embodiments, Ring B is furyl (e.g., fur-2-yl, fur-3-yl). In some embodiments, Ring B is thiazolyl (e.g., thiazol-2-yl, thiazol-4-yl, thiazol-5-yl). In some embodiments, Ring B is isothiazolyl (e.g., isothiazol-3-yl, isothiazol-4-yl, isothiazol-5-yl). In some embodiments, Ring B is oxazolyl (e.g., oxazol-2-yl, oxazol-4-yl, oxazol-5-yl). In some embodiments, Ring B is isoxazolyl (e.g., isoxazol-3-yl, isoxazol-4-yl, isoxazol-5-yl). In some embodiments, Ring B is imidazolyl (e.g., imidazol-2-yl, imidazol-4-yl). In some embodiments, Ring B is triazolyl. In some embodiments, Ring B is thiadiazolyl. In some embodiments, Ring B is oxadiazolyl. In some embodiments, Ring B is pyrazol-5-yl. In some embodiments, Ring B is pyrazol-3-yl. In some embodiments, Ring B is thiophen-3-yl. In some embodiments, Ring B is oxazol-5-yl.

In certain embodiments, the 5-membered monocyclic heteroaryl is unsubstituted. In some embodiments, the 5-membered monocyclic heteroaryl is substituted with 1 instance of R³. In some embodiments, the 5-membered monocyclic heteroaryl is substituted with 2 instances of R³. In some embodiments, the 5-membered monocyclic heteroaryl is substituted with 3 instances of R³.

In some embodiments, Ring B is a 6-membered monocyclic heteroaryl (e.g., pyridyl, pyrimidinyl, triazinyl, pyrazinyl, pyridazinyl). In some embodiments, the 6-membered monocyclic heteroaryl is unsubstituted. In some embodiments, the 6-membered monocyclic heteroaryl is substituted with 0, 1, 2 or 3 instances of R³. In some embodiments, the 6-membered monocyclic heteroaryl is substituted with 1 instance of R³. In some embodiments, the 6-membered monocyclic heteroaryl is substituted with 2 instances of R³. In some embodiments, the 6-membered monocyclic heteroaryl is substituted with 3 instances of R³. In some embodiments, Ring B is pyridinyl (e.g., pyridin-2-yl, pyridin-3-yl, pyridin-4-yl). In some embodiments, Ring B is pyridin-2-yl. In some embodiments, Ring B is pyridin-3-yl. In some embodiments, Ring B is pyridin-4-yl. In some embodiments, Ring B is pyrimidinyl (e.g, pyrimidin-2-yl, pyrimidin-4-yl, pyrimidin-5-yl).

In some embodiments, Ring B is selected from the group consisting of:

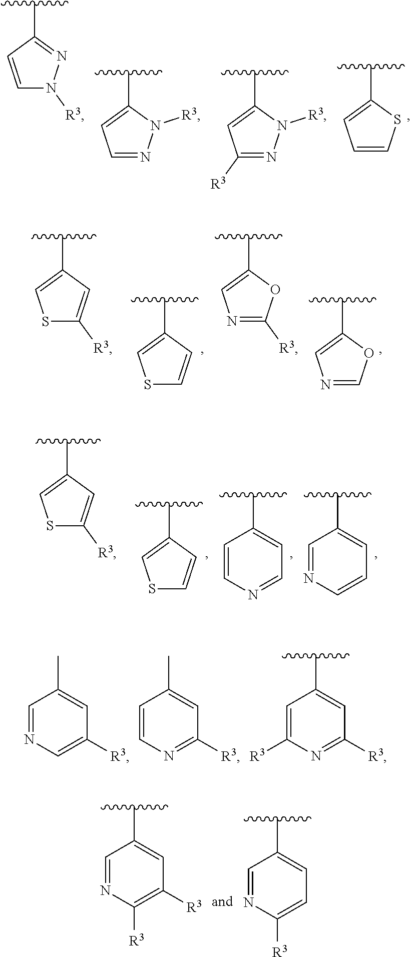

wherein each R³ is independently as defined in any of the embodiments described herein.

In some embodiments, Ring B is

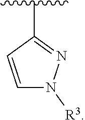

In some embodiments, Ring B is

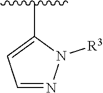

wherein R³ is as defined in any of the embodiments described herein. In some embodiments, Ring B is

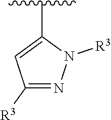

wherein R³ is as defined in any of the embodiments described herein. In some embodiments, Ring B is

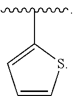 [SMILES: *c1cccs1]

In some embodiments, Ring B is

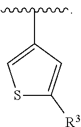

wherein R³ is as defined in any of the embodiments described herein. In some embodiments, Ring B is

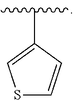 [SMILES: *c1ccsc1]

In some embodiments, Ring B is

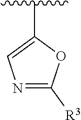

wherein R³ is as defined in any of the embodiments described herein. In some embodiments, Ring B is

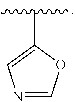 [SMILES: *c1cnco1]

In some embodiments, Ring B is

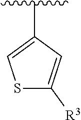

wherein R³ is as defined in any of the embodiments described herein. In some embodiments, Ring B is

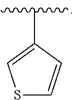 [SMILES: *c1ccsc1]

In some embodiments, Ring B is

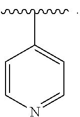 [SMILES: *c1ccncc1]

In some embodiments, Ring B is

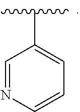 [SMILES: *c1cccnc1]

In some embodiments, Ring B is

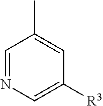

wherein R³ is as defined in any of the embodiments described herein. In some embodiments, Ring B is

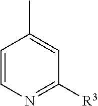

wherein R³ is as defined in any of the embodiments described herein. In some embodiments, Ring B is

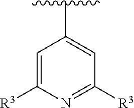

wherein R³ is as defined in any of the embodiments described herein. In some embodiments, Ring B is

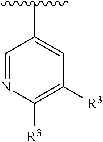

wherein R³ is as defined in any of the embodiments described herein. In some embodiments, Ring B is

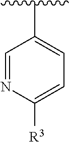

wherein R³ is as defined in any of the embodiments described herein.

In some embodiments, Ring B is an 8-10 membered bicyclic heteroaryl, wherein the bicyclic heteroaryl is optionally substituted (e.g., substituted with 0, 1, 2 or 3 instances of R³ wherein R³ is as defined in any of the embodiments described herein). In certain embodiments, Ring B is an 8-10 membered bicyclic heteroaryl (e.g., a 5,5 bicyclic heteroaryl (e.g., 1H-thieno[2,3-c]pyrazolyl, 1H-thieno[3,2-c]pyrazolyl), a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl), 1H-pyrazolo[3,4-b]pyridinyl, thiazolo[5,4-b]pyridinyl), or a 6,6 bicyclic heteroaryl (e.g., quinolinyl, 1,5-naphthyridinyl, 1,2-dihydro-1,5-naphthyridinyl, 1,2,3,4-tetrahydro-1,8-naphthyridinyl, isoquinolinyl), wherein each bicyclic heteroaryl contains 1, 2, 3 or 4 heteroatoms independently selected from the group consisting of O, N and S, and wherein each bicyclic heteroaryl is optionally substituted (e.g., substituted with 0, 1, 2 or 3 instances of R³ wherein R³ is as defined in any of the embodiments described herein). In some embodiments, Ring B is a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl), 1H-pyrazolo[3,4-b]pyridinyl, thiazolo[5,4-b]pyridinyl) or a 6,6 bicyclic heteroaryl (e.g., quinolinyl, isoquinolinyl, 1,5-naphthyridinyl, 1,2-dihydro-1,5-naphthyridinyl, 1,2,3,4-tetrahydro-1,8-naphthyridinyl, isoquinolinyl).

In some embodiments, Ring B is a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl), 1H-pyrazolo[3,4-b]pyridinyl, thiazolo[5,4-b]pyridinyl).

In some embodiments, ring B is a 6,6 bicyclic heteroaryl (e.g., quinolinyl, isoquinolinyl, 1,5-naphthyridinyl, 1,2-dihydro-1,5-naphthyridinyl, 1,2,3,4-tetrahydro-1,8-naphthyridinyl, isoquinolinyl).

In some embodiments, the bicyclic heteroaryl (e.g., the 5,5 bicyclic heteroaryl, 5,6 bicyclic heteroaryl, 6,6 bicyclic heteroaryl) contains 1, 2 or 3 heteroatoms selected from the group consisting of O, N and S. In some embodiments, the bicyclic heteroaryl contains 1 or 2 heteroatoms selected from the group consisting of O, N and S. In some embodiments, the bicyclic heteroaryl contains 1 heteroatom selected from the group consisting of O, N and S.

In some embodiments, the bicyclic heteroaryl contains 2 heteroatoms selected from the group consisting of O, N and S. In some embodiments, the bicyclic heteroaryl contains 3 heteroatoms selected from the group consisting of O, N and S. In some embodiments, the bicyclic heteroaryl contains 4 heteroatoms selected from the group consisting of O, N and S.

In some embodiments Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted (e.g., substituted with 0, 1, 2 or 3 instances of R³).

In certain embodiments, Ring B is selected from the group consisting of 1H-indazolyl, 2H-indazolyl, quinolinyl, isoquinolinyl and benzo[d]thiazolyl, each optionally substituted (e.g., substituted with 0, 1, 2 or 3 instances of R³).

In certain embodiments, Ring B is selected from the group consisting of 2H-indazolyl, quinolinyl, isoquinolinyl and benzo[d]thiazolyl, each optionally substituted (e.g., substituted with 0, 1, 2 or 3 instances of R³). In some embodiments, Ring B is 1H indazolyl, optionally substituted at any available position (e.g., substituted with 0, 1, 2 or 3 instances of R³, wherein R³ is as defined in any of the embodiments described herein).

In some embodiments, Ring B is 2H-indazolyl, optionally substituted at any available position (e.g., substituted with 0, 1, 2 or 3 instances of R³, wherein R³ is as defined in any of the embodiments described herein).

In some embodiments, Ring B is quinolinyl, optionally substituted at any available position (e.g., substituted with 0, 1, 2 or 3 instances of R³, wherein R³ is as defined in any of the embodiments described herein).

In some embodiments, Ring B is isoquinolinyl, optionally substituted at any available position (e.g., substituted with 0, 1, 2 or 3 instances of R³, wherein R³ is as defined in any of the embodiments described herein).

In some embodiments, Ring B is and benzo[d]thiazolyl, optionally substituted at any available position (e.g., substituted with 0, 1, 2 or 3 instances of R³, wherein R³ is as defined in any of the embodiments described herein).

In some embodiments, Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each optionally substituted (e.g., substituted with 0, 1, 2 or 3 instances of R³).

In some embodiments, Ring B is independently selected from the group consisting of 1H-indazol-5-yl, 1H indazol-4-yl, 2H-indazol-6-yl, 2H-indazol-5-yl, quinolin-6-yl, quinolin-7-yl, isoquinolin-6-yl and benzo[d]thiazol-5-yl, each optionally substituted at any available position (e.g., substituted with 0, 1, 2 or 3 instances of R³, wherein R³ is as defined in any of the embodiments described herein).

In some embodiments, Ring B is independently selected from the group consisting of 1H-indazol-5-yl, 2H-indazol-6-yl, 2H-indazol-5-yl, quinolin-6-yl, quinolin-7-yl, isoquinolin-6-yl and benzo[d]thiazol-5-yl, each optionally substituted (e.g., substituted with 0, 1, 2 or 3 instances of R³). In some embodiments, Ring B is optionally substituted 2H-indazol-6-yl (e.g., substituted with 0, 1, 2 or 3 instances of R³). In some embodiments, Ring B is independently selected from the group consisting of 2H-indazol-6-yl, 2H-indazol-5-yl, quinolin-6-yl, quinolin-7-yl, isoquinolin-6-yl and benzo[d]thiazol-5-yl, each optionally substituted (e.g., substituted with 0, 1, 2 or 3 instances of R³). In some embodiments, Ring B is optionally substituted 1H-indazol-5-yl (e.g., substituted with 0, 1, 2 or 3 instances of R³). In some embodiments, Ring B is optionally substituted 1H indazol-4-yl (e.g., substituted with 0, 1, 2 or 3 instances of R³).

In some embodiments, Ring B is optionally substituted 2H-indazol-6-yl (e.g., substituted with 0, 1, 2 or 3 instances of R³). In some embodiments, Ring B is optionally substituted 2H-indazol-5-yl (e.g., substituted with 0, 1, 2 or 3 instances of R³). In some embodiments, Ring B is optionally substituted quinolin-6-yl (e.g., substituted with 0, 1, 2 or 3 instances of R³). In some embodiments, Ring B is optionally substituted quinolin-7-yl (e.g., substituted with 0, 1, 2 or 3 instances of R³). In some embodiments, Ring B is optionally substituted isoquinolin-6-yl (e.g., substituted with 0, 1, 2 or 3 instances of R³). In some embodiments, Ring B is optionally substituted benzo[d]thiazol-5-yl (e.g., substituted with 0, 1, 2 or 3 instances of R³).

In some embodiments Ring B is an 8-10 membered bicyclic heteroaryl selected from the group consisting of:

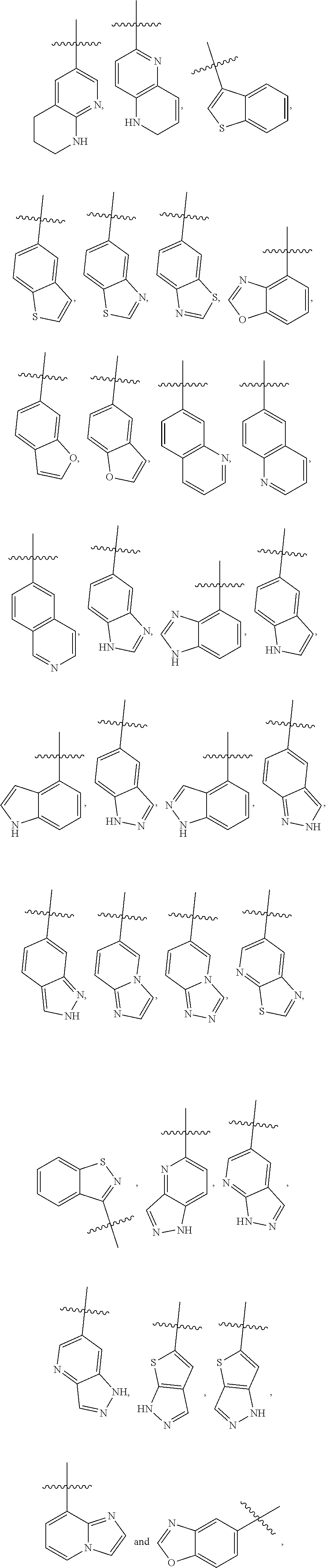

each optionally substituted (e.g., substituted with 0, 1, 2 or 3 instances of R³).

In certain embodiments, Ring B is selected from the group consisting of:

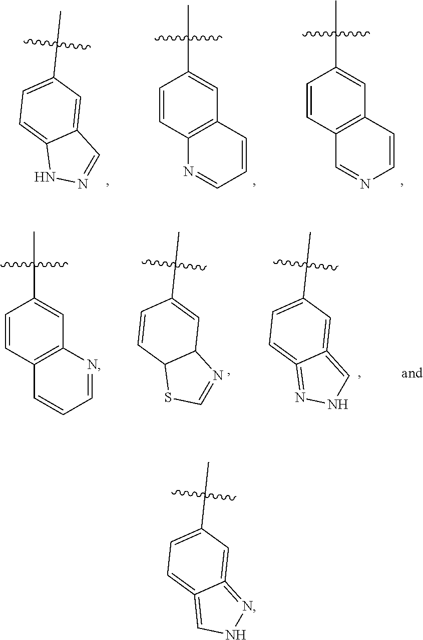

each optionally substituted (e.g., substituted with 0, 1, 2 or 3 instances of R³).

In certain embodiments, Ring B is selected from the group consisting of:

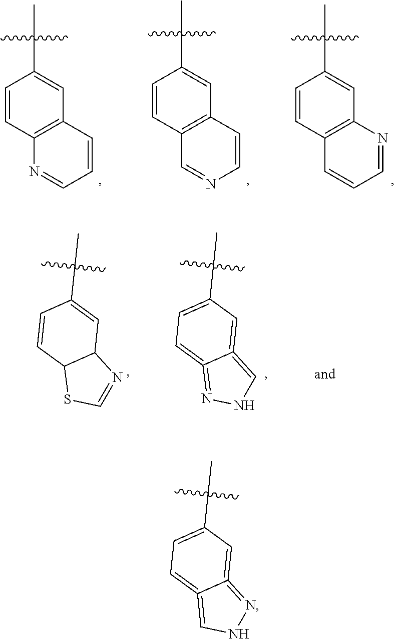

each optionally substituted (e.g., substituted with 0, 1, 2 or 3 instances of R³).

In some embodiments, the 8-10 membered bicyclic heteroaryl is unsubstituted. In some embodiments, the 8-10 membered bicyclic heteroaryl is substituted with 1 instance of R³. In some embodiments, the 8-10 membered bicyclic heteroaryl is substituted with 2 instances of R³. In some embodiments, 8-10 membered bicyclic heteroaryl is substituted with 3 instances of R³.

In some embodiments, Ring B is selected from the group consisting of:

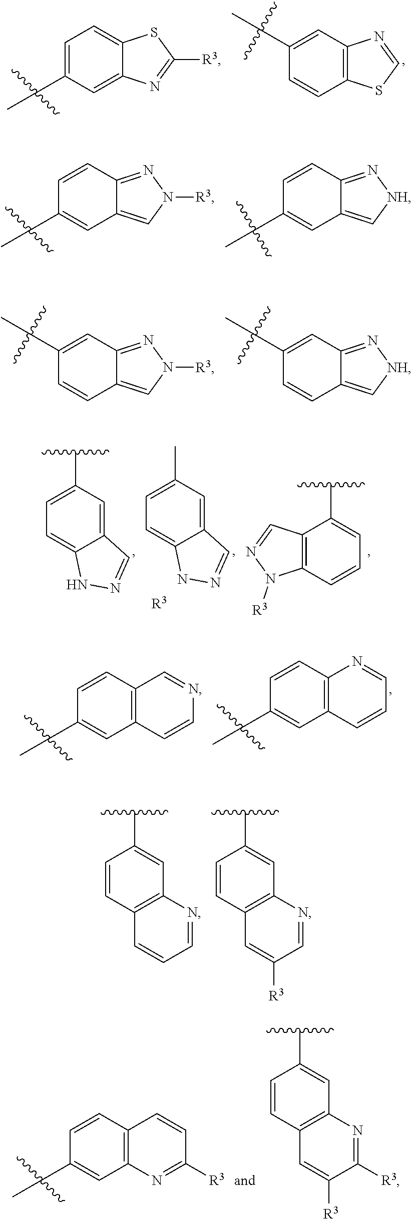

wherein each R³ is independently as defined in any of the embodiments described herein.

In certain embodiments, Ring B is selected from the group consisting of:

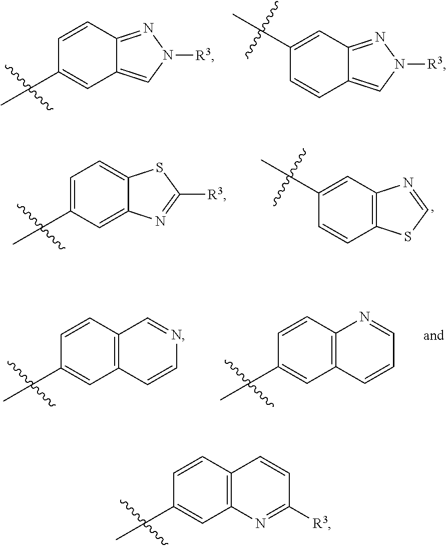

wherein R³ is as defined in any of the embodiments described herein.

In certain embodiments, Ring B is selected from the group consisting of:

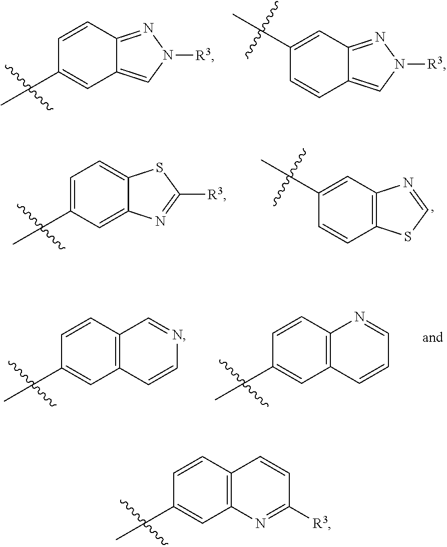

wherein R³ is as defined in any of the embodiments described herein.

In some embodiments, Ring B is selected from the group consisting of: Ring B is selected from the group consisting of:

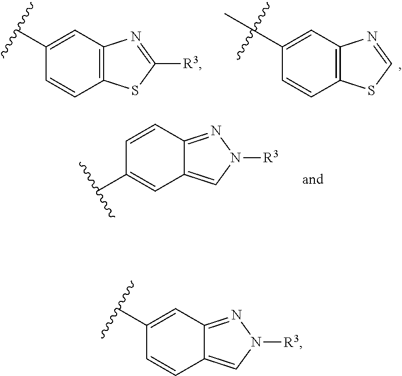

wherein R³ is as defined in any of the embodiments described herein. In some embodiments, Ring B is selected from the group consisting of:

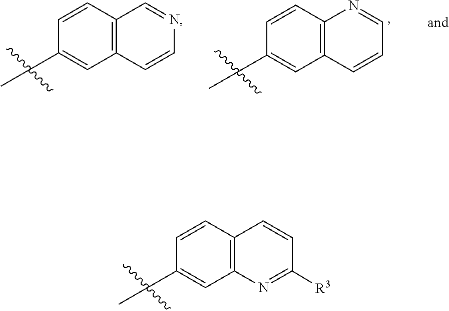

wherein R³ is as defined in any of the embodiments described herein.

In some embodiments, Ring B is selected from the group consisting of:

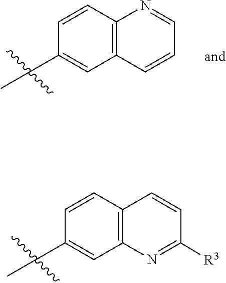

wherein R³ is as defined in any of the embodiments described herein.

In certain embodiments, Ring B is selected from the group consisting of:

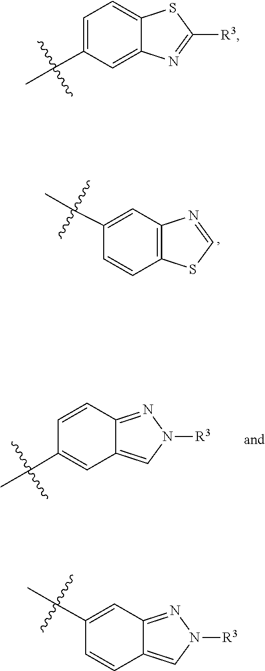

wherein R³ is as defined in any of the embodiments described herein.

In some embodiments, Ring B is selected from the group consisting of:

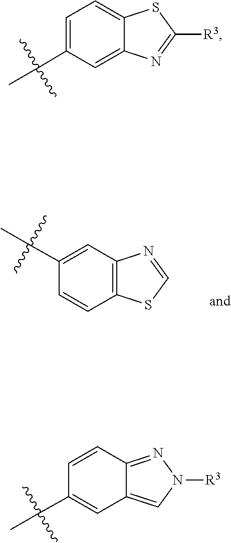

wherein R³ is as defined in any of the embodiments described herein.

In some embodiments, Ring B is wherein R³ is as defined in any of the embodiments described herein

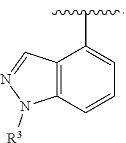

In some embodiments, Ring B is

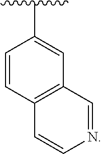 [SMILES: *c1ccc2ccncc2c1]

In some embodiments, Ring B is

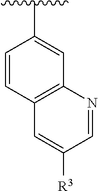

wherein R³ is as defined in any of the embodiments described herein. In some embodiments, Ring B is

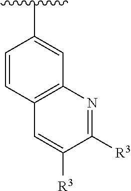

wherein R³ is as defined in any of the embodiments described herein. In certain embodiments, Ring B is

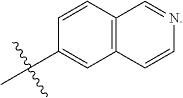 [SMILES: *c1ccc2cnccc2c1]

In certain embodiments, Ring B is

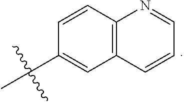 [SMILES: *c1ccc2ncccc2c1]

In certain embodiments, Ring B is

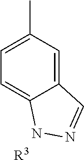

wherein R³ is as defined in any of the embodiments described herein. In certain embodiments, Ring B is

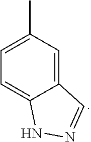

In certain embodiments, Ring B is

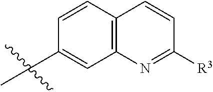

wherein R³ is as defined in any of the embodiments described herein.

In certain embodiments, Ring B is

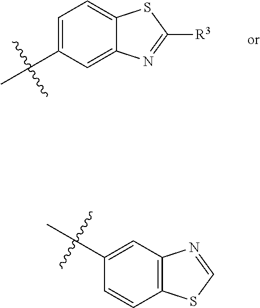

wherein R³ is as defined in any of the embodiments described herein. In some embodiments, Ring B is

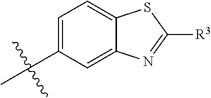

wherein R³ is as defined in any of the embodiments described herein. In other embodiments, Ring B is

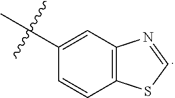 [SMILES: *c1ccc2scnc2c1]

In certain embodiments, Ring B is

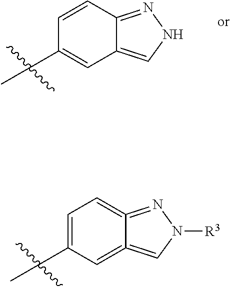

wherein R³ is as defined in any of the embodiments described herein.

In certain embodiments, Ring B is

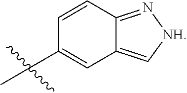 [SMILES: *c1ccc2n[nH]cc2c1]

In certain embodiments, Ring B is

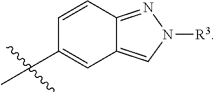

wherein R³ is as defined in any of the embodiments described herein.

In certain embodiments, Ring B is

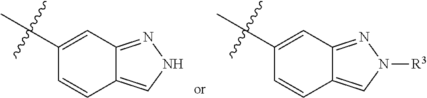

wherein R³ is as defined in any of the embodiments described herein.

In certain embodiments, Ring B is

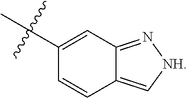 [SMILES: *c1ccc2c[nH]nc2c1]

In certain embodiments, Ring B is

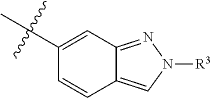

wherein R³ is as defined in any of the embodiments described herein.

In some embodiments, the compounds of Formula (I) are of Formula (III2):

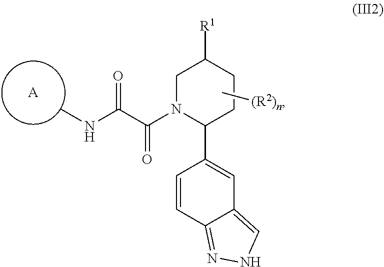

wherein Ring A, R¹, R² and n are as defined in any of the embodiments described herein and the 2H indazole is substituted with 0, 1, 2 or 3 instances of R³ as defined in any of the embodiments described herein. In some embodiments, the compounds of Formula (I) are of Formula (III2) a:

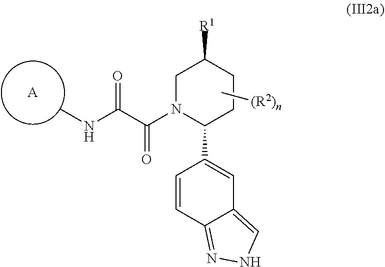

wherein Ring A, R¹, R² and n are as defined in any of the embodiments described herein and the 2H indazole is substituted with 0, 1, 2 or 3 instances of R³ as defined in any of the embodiments described herein. In yet some embodiments, compounds of Formula (I) are of Formula (III2b),

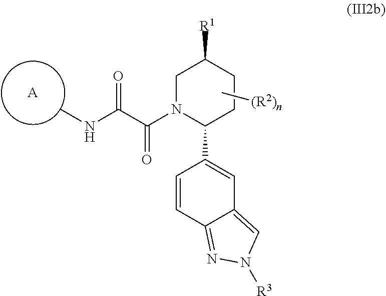

wherein Ring A, R¹, R² R³ and n are as defined in any of the embodiments described herein.

In some embodiments, the compounds of Formula (I) are of Formula (III3):

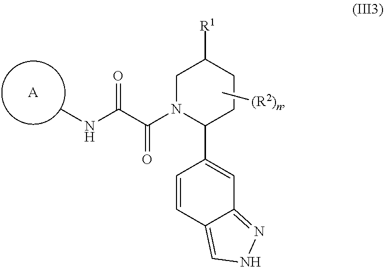

wherein Ring A, R¹, R² and n are as defined in any of the embodiments described herein and the 2H indazole is substituted with 0, 1, 2 or 3 instances of R³ as defined in any of the embodiments described herein. In some embodiments, the compounds of Formula (I) are of Formula (III3a):

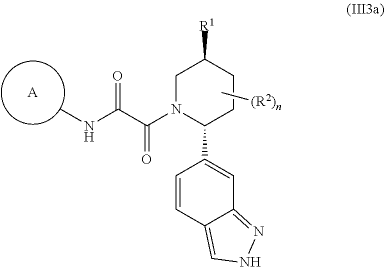

wherein Ring A, R¹, R² and n are as defined in any of the embodiments described herein and the 2H indazole is substituted with 0, 1, 2 or 3 instances of R³ as defined in any of the embodiments described herein. In yet some embodiments, compounds of Formula (I) are of Formula (III3b),

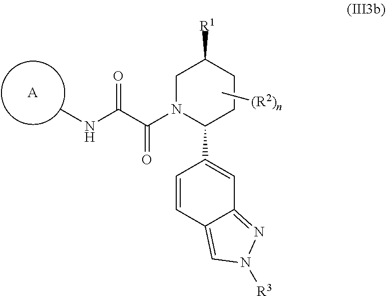

wherein Ring A, R¹, R² R³ and n are as defined in any of the embodiments described herein.

In some embodiments, the compounds of Formula (I) are of Formula (III4):

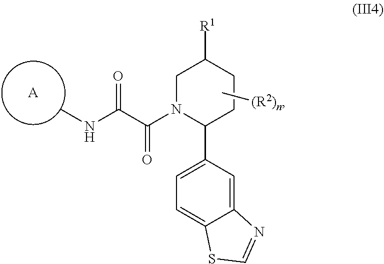

wherein Ring A, R¹, R² and n are as defined in any of the embodiments described herein and the benzothiazole is substituted with 0, 1, 2 or 3 instances of R³ as defined in any of the embodiments described herein. In some embodiments, the compounds of Formula (I) are of Formula (III4a):

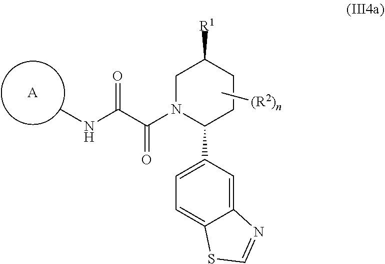

wherein Ring A, R¹, R² and n are as defined in any of the embodiments described herein and the benzothiazole is substituted with 0, 1, 2 or 3 instances of R³ as defined in any of the embodiments described herein. In yet some embodiments, compounds of Formula (I) are of Formula (III4b),

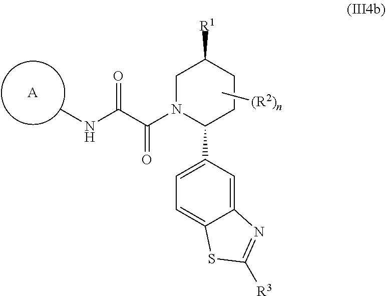

wherein Ring A, R¹, R² R³ and n are as defined in any of the embodiments described herein.

In some embodiments, the compounds of Formula (I) are of Formula (III4c):

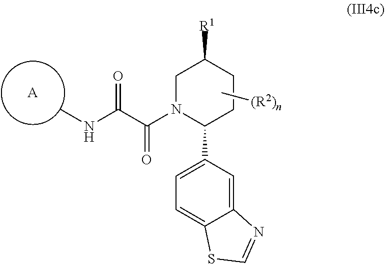

wherein Ring A, R¹, R² and n are as defined in any of the embodiments described herein and the benzothiazole is unsubstituted.

In some embodiments, the compounds of Formula (I) are of Formula (III5):

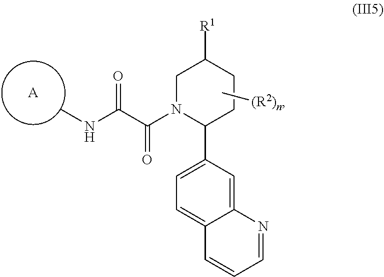

wherein Ring A, R¹, R² and n are as defined in any of the embodiments described herein and the quinoline is substituted with 0, 1, 2 or 3 instances of R³ as defined in any of the embodiments described herein. In some embodiments, the compounds of Formula (I) are of Formula (III5a):

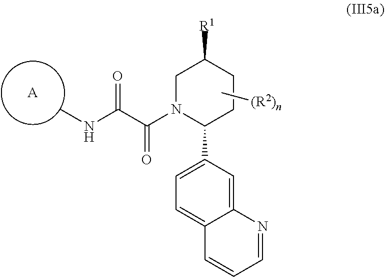

wherein Ring A, R¹, R² and n are as defined in any of the embodiments described herein and the quinoline is substituted with 0, 1, 2 or 3 instances of R³ as defined in any of the embodiments described herein. In yet some embodiments, compounds of Formula (I) are of Formula (III5b),

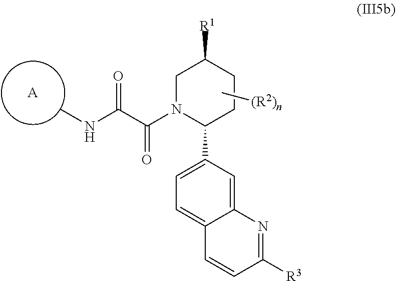

wherein Ring A, R¹, R² R³ and n are as defined in any of the embodiments described herein.

In some embodiments, the compounds of Formula (I) are of Formula (III6):

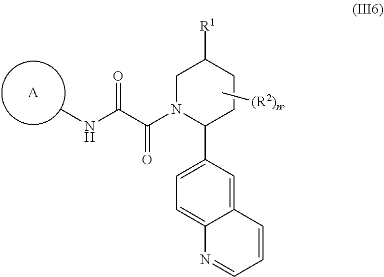

wherein Ring A, R¹, R² and n are as defined in any of the embodiments described herein and the quinoline is substituted with 0, 1, 2 or 3 instances of R³ as defined in any of the embodiments described herein. In some embodiments, the compounds of Formula (I) are of Formula (III6a):

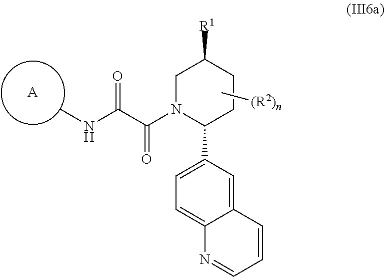

wherein Ring A, R¹, R² and n are as defined in any of the embodiments described herein and the quinoline is substituted with 0, 1, 2 or 3 instances of R³ as defined in any of the embodiments described herein. In yet some embodiments, the quinoline is unsubstituted.

In some embodiments, the compounds of Formula (I) are of Formula (III7):

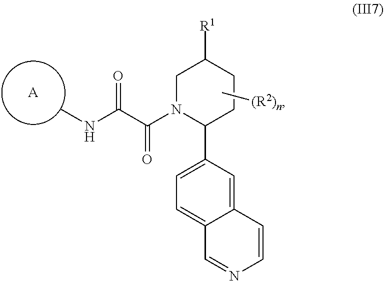

wherein Ring A, R¹, R² and n are as defined in any of the embodiments described herein and the isoquinoline is substituted with 0, 1, 2 or 3 instances of R³ as defined in any of the embodiments described herein. In some embodiments, the compounds of Formula (I) are of Formula (III7a):

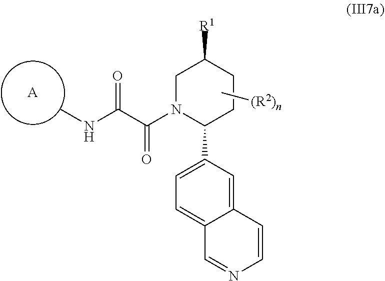

wherein Ring A, R¹, R² and n are as defined in any of the embodiments described herein and the isoquinoline is substituted with 0, 1, 2 or 3 instances of R³ as defined in any of the embodiments described herein. In yet some embodiments, the isoquinoline is unsubstituted.

As generally defined herein, each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂, wherein R^{a1} is as defined in any of the embodiments described herein.

In some embodiments, each R¹ is independently selected from the group consisting of H, halo (e.g., —F, —C₁), —CN, —C₁-C₆ alkyl (e.g., -Me, -Et, -Pr, -ⁱPr, -sec-Bu, -^{t}Bu), 5-membered heteroaryl (e.g., pyrazolyl), —C₁-C₆ haloalkyl (e.g., —CF₃, —CHF₂, —CH₂CF₃), —C₁-C₆ heteroalkyl (e.g., —CH₂OH), —C₃-C₉ cycloalkyl (e.g., cyclopropyl, cyclobutyl, cyclopentyl, cyclohexyl), —OR^{a1} (e.g., —OH, —OCH₃, —OCHF₂), —N(R^{a1})₂ and —C(═O)N(R^{a1})₂ (e.g., —C(═O)NH₂, —C(═O)NHCH₃), wherein each R^{a1} is as defined in any of the embodiments described herein. In some embodiments, each R^{a1} is independently selected from the group consisting of H and —C₁-C₆ alkyl (e.g., -Me, -Et, -Pr, -ⁱPr, -ⁿBu, -^{t}Bu, -sec-Bu, -iso-Bu).

In some embodiments, each R¹ is independently selected from the group consisting of H, -Me and -Et.

In certain embodiments, each R¹ is independently selected from the group consisting of H and methyl.

In some embodiments, R¹ is H. In some embodiments R¹ is -D.

In certain embodiments, R¹ is halo (e.g., fluoro, chloro, bromo, iodo). In some embodiments, R¹ is —Cl. In some embodiments, R¹ is —F. In some embodiments, R¹ is —Br. In some embodiments, R¹ is —I.

In some embodiments, R¹ is —CN.

In certain embodiments, R¹ is —C₁-C₆ alkyl. In some embodiments, R¹ is selected from -Me and -Et. In some embodiments, R¹ is -Me. In some embodiments, R¹ is -Et. In some embodiments R¹ is -Pr or -iPr.

In some embodiments, R¹ is —C₁-C₆ heteroalkyl. In some embodiments, R¹ is methoxymethyl (—CH₂OCH₃). In some embodiments, R¹ is hydroxymethyl (—CH₂OH). In some embodiments, R¹ is aminomethyl (e.g., —CH₂NH₂, —CH₂NHCH₃, —CH₂N(CH₃)₂.

In some embodiments, R¹ is —C₁-C₆ haloalkyl. In some embodiments, R¹ is trifluoromethyl (—CF₃). In other embodiments, R¹ is difluoromethyl (—CHF₂).

In some embodiments, R¹ is C₃-C₉ cycloalkyl (e.g., cyclopropyl, cyclobutyl, cyclopentyl, cyclohexyl). In some embodiments, R¹ is cyclopropyl. In some embodiments R¹ is cyclobutyl. In some embodiments, R¹ is cyclopentyl. In some embodiments, R¹ is cyclohexyl.

In some embodiments, R¹ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R¹ is oxetanyl. In some embodiments, R¹ is tetrahydropyranyl. In some embodiments, R¹ is tetrahydrofuranyl. In some embodiments, R¹ is azetidinyl. In some embodiments, R¹ is pyrrolidinyl. In some embodiments, R¹ is piperidinyl. In some embodiments, R¹ is piperazinyl. In some embodiments, R¹ is morpholinyl. In some embodiments, R¹ is azepanyl.

In some embodiments, R¹ is a 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms). In some embodiments, R¹ is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R¹ is thiophenyl (e.g., thiophen-2-yl, thiophen-3-yl). In some embodiments, R¹ is pyrazolyl (e.g., pyrazol-1-yl, pyrazol-3-yl, pyrazol-5-yl). In some embodiments, R¹ is thiazolyl (e.g., thiazol-2-yl, thiazol-4-yl, thiazol-5-yl). In some embodiments, R¹ is a 6-membered monocyclic heteroaryl (e.g., pyridyl, pyrimidinyl, triazinyl, pyrazinyl, pyridazinyl). In some embodiments, R¹ is pyridinyl (e.g., pyridin-2-yl, pyridin-3-yl, pyridin-4-yl). In some embodiments, R¹ is pyrimidinyl (e.g., pyrimidin-2-yl, pyrimidin-4-yl, pyrimidin-5-yl).

In some embodiments R¹ is cycloalkylalkyl (e.g., cyclopropylmethyl, cyclobutylmethyl, cyclopentylmethyl, cyclohexylmethyl, cycloheptylmethyl).

In some embodiments, R¹ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R¹ is arylalkyl. In some embodiments, R¹ is benzyl.

In some embodiments, R¹ is heteroarylalkyl (e.g., pyridinylmethyl, thiazolylmethyl, triazolylmethyl, pyrazolylmethyl).

In some embodiments, R¹ is —OR^{a1} wherein R^{a1} is as described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R¹ is hydroxy. In some embodiments, R¹ is methoxy. In some embodiments, R¹ is ethoxy. In some embodiments, R¹ is propoxy. In some embodiments, R¹ is isopropoxy. In some embodiments R¹ is difluoromethoxy. (—OCHF₂). In some embodiments, R¹ is trifluoromethoxy (—OCF₃).

In some embodiments, R¹ is —N(R^{a1})₂ (e.g., —NH₂, —NHR^{a1}, —N(CH₃)R^{a1}). In some embodiments, R¹ is —NH₂. In some embodiments, R¹ is —NHR^{a1} (e.g., —NHCH₃, —NHCH₂CH₃, —NHPr, —NHⁱPr, —NHcyclopropyl, —NHcyclobutyl). In some embodiments, R¹ is —N(CH₃)R^{a1} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R¹ is —C(═O)R^{a1} or —C(═O)OR^{a1} wherein R^{a1} is as described herein. In some embodiments, R¹ is —C(═O)R^{a1} wherein R^{a1} is as described herein. In some embodiments, R¹ is —C(═O)alkyl. In some embodiments, R¹ is —C(═O)CH₃, —C(═O)cyclopropyl, —C(═O)cyclobutyl, —C(═O)^{t}Bu, —C(═O)ⁱPr, —C═(O)CH₂CH₂CH₃, or —C(═O)OCH₃. In some embodiments, R¹ is acetyl (—C(═O)CH₃). In some embodiments, R¹ is —C(═O)OR^{a1}. In some embodiments, R¹ is —COOH. In some embodiments, R¹ is COOCH₃.

In some embodiments, R¹ is —NR^{a1}C(═O)R^{a1} wherein R^{a1} is as described herein. In certain embodiments, R¹ is —NHC(═O)R^{a1} (e.g., —NHC(═O)CH₃, —NHC(═O)CH₂CH₃, —NHC(═O)CH₂CH₂CH₃, —NHC(═O)ⁱPr, —NHC(═O)^{t}Bu, —NHC(═O)^{t}Bu, —NHC(═O)Cyclopropyl, —NHC(═O)Cyclobutyl). In some embodiments, R¹ is —N(CH₃)C(═O)R^{a1} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R¹ is —NR^{a1}C(═O)OR^{a1} wherein R^{a1} is as described herein. In certain embodiments, R¹ is —NHC(═O)OR^{a1} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R¹ is —N(CH₃)C(═O)OR^{a1} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R¹ is —C(═O)N(R^{a1})₂ (e.g., —C(═O)NH₂, —C(═O)NHR^{a1}, —C(═O)N(CH₃)R^{a1}). In some embodiments, R¹ is —C(═O)NH₂. In certain embodiments, R¹ is —C(═O)NHR^{a1} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R¹ is —C(═O)N(CH₃)R^{a1} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)^{t}Bu, —C(═O)N(CH₃)^{t}Bu, —C(═O)N(CH₃)Cyclopropyl, —C(═O)N(CH₃)Cyclobutyl).

In some embodiments, R¹ is —OC(═O)N(R^{a1})₂ wherein R^{a1} is as described herein. In certain embodiments, R¹ is —OC(═O)NHR^{a1} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R¹ is —OC(═O)N(CH₃)R^{a1} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)^{t}Bu, —OC(═O)N(CH₃)^{t}Bu, —OC(═O)N(CH₃)Cyclopropyl, —OC(═O)N(CH₃)Cyclobutyl).

In some embodiments, R¹ is —S(═O)R^{a1} wherein R^{a1} is as described herein. In certain embodiments, R¹ is —S(═O)alkyl (e.g., —S(═O)CH₃, —S(═O)CH₂CH₃, —S(═O)CH₂CH₂CH₃, —S(═O)ⁱPr). In certain embodiments, R¹ is —S(═O)cycloalkyl (e.g., —S(═O)cyclopropyl, —S(═O)cyclobutyl, —S(═O)cyclopentyl, —S(═O)cyclohexyl).

In some embodiments, R¹ is —S(═O)₂R^{a1} wherein R^{a1} is as described herein. In certain embodiments, R¹ is —S(═O)₂alkyl (e.g., —S(═O)₂CH₃, —S(═O)₂CH₂CH₃, —S(═O)₂Pr, —S(═O)₂ⁱPr). In certain embodiments, R¹ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R¹ is S(═O)₂aryl (e.g., —S(═O)₂phenyl).

In some embodiments, R¹ is —SR^{a1} wherein R^{a1} is as described herein. In certain embodiments, R¹ is -Salkyl (e.g., —SCH₃, —SCH₂CH₃, —SPr, —SⁱPr). In certain embodiments, R¹ is -Scycloalkyl (e.g., -Scyclopropyl, —Scyclobutyl, —Scyclopentyl, —Scyclohexyl). In certain embodiments, R¹ is -Saryl (e.g., -Sphenyl).

In some embodiments, R¹ is —S(═O)(═NR^{a1})R^{a1} wherein R^{a1} is as described herein. In certain embodiments, R¹ is —S(═O)(═NH)R^{a1} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)^{t}Bu, —S(═O)(═NH)Bu, —S(═O)(═NH). Cyclopropyl, —S(═O)(═NH)Cyclobutyl). In some embodiments, R¹ is —S(═O)(═NCH₃)R^{a1} (e.g., —S(═O)(═NCH₃)CH₃, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R¹ is —NR^{a1}S(═O)₂R^{a1} wherein R^{a1} is as described herein. In certain embodiments, R¹ is —NHS(═O)₂alkyl (e.g., —NHS(═O)₂CH₃, —NHS(═O)₂CH₂CH₃, —NHS(═O)₂Pr, —NHS(═O)₂ⁱPr). In certain embodiments, R¹ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R¹ is —N(CH₃) S(═O)₂alkyl (e.g., —N(CH₃) S(═O)₂CH₃, —N(CH₃) S(═O)₂CH₂CH₃, —N(CH₃) S(═O)₂Pr, —N(CH₃) S(═O)₂ⁱPr). In certain embodiments, R¹ is —N(CH₃) S(═O)₂cycloalkyl (e.g., —N(CH₃) S(═O)₂cyclopropyl, —N(CH₃) S(═O)₂cyclobutyl, —N(CH₃) S(═O)₂cyclopentyl, —N(CH₃) S(═O)₂cyclohexyl).

In some embodiments, R¹ is —S(═O)₂N(R^{a1})₂ wherein R^{a1} is as described herein. (e.g., —S(═O)₂NH₂, —S(═O)₂NHR^{a1}, —S(═O)₂N(CH₃)R^{a1}). In some embodiments, R¹ is —S(═O)₂NH₂. In some embodiments, R¹ is —S(═O)₂NHR^{a1} (e.g., —S(═O)₂NHCH₃, —S(═O)₂NHCH₂CH₃, —S(═O)₂NHPr, —S(═O)₂NHⁱPr, —S(═O)₂NHcyclopropyl, —S(═O)₂NHcyclobutyl). In some embodiments, R¹ is —S(═O)₂N(CH₃)R^{a1} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring), wherein R^{a2} is as defined in any of the embodiments described herein.

In some embodiments of Formula A or Formula (I), two R² groups are taken together with the atom to which they are attached to form a 3-10 membered spiro cycloalkyl (e.g., cyclopropyl, cyclobutyl, cyclopentyl, cyclohexyl, cycloheptyl) or spiro heterocyclyl ring (e.g., oxetanyl, tetrahydrofuranyl, tetrahydropyranyl, azetidinyl, pyrrolidinyl, piperidinyl, piperazinyl, morpholinyl, azepanyl, tetrahydrothiopyranyl, thiomorpholinyl).

In some embodiments of Formula (A) or Formula (I), two R² groups are taken together with the adjacent atoms to which they are attached to form a 3-10 membered fused cycloalkyl (e.g., cyclopropyl, cyclobutyl, cyclopentyl, cyclohexyl, cycloheptyl) or fused heterocyclyl ring (e.g., oxetanyl, tetrahydrofuranyl, tetrahydropyranyl, azetidinyl, pyrrolidinyl, piperidinyl, piperazinyl, morpholinyl, azepanyl, tetrahydrothiopyranyl, thiomorpholinyl).

In some embodiments, the two R² groups taken together with the atoms to which they are attached form a bridged piperidine-containing carbocyclyl or heterocyclyl ring (e.g., 2-azabicyclo[2.2.1]heptane, 2-azabicyclo[2.2.2]octane, 3-azabicyclo[3.2.1]octane).

In other embodiments, the R² groups are not taken together to form cycloalkyl or heterocyclyl rings (i.e., each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂, wherein R^{a2} is as defined in any of the embodiments described herein.

In some embodiments, each R² is independently selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl, cyclobutyl), 3-6 membered heterocyclyl (e.g., oxetanyl, tetrahydrofuranyl), —OR^{a2}, —N(R^{a2})₂, —C(═O)R^{a2}, —C(═O)OR^{a2}, —NR^{a4}C(═O)R^{a2}, —NR^{a2}C(═O)OR^{a2}, —C(═O)N(R^{a2})₂, —OC(═O)N(R^{a2})₂, wherein each R^{a2} is as defined in any of the embodiments described herein. In some embodiments, each R² is independently selected from the group consisting of halo, —CN, —C₁-C₆ alkyl, —C₁-C₆ haloalkyl, —C₃-C₉ cycloalkyl (e.g., cyclopropyl), 3-6 membered heterocyclyl (e.g., oxetanyl, tetrahydrofuranyl), —OR^{a2}, —N(R^{a2})₂, —C(═O)R^{a2}, —C(═O)N(OR^{a2})(R^{a2}), and —C(═O)N(R^{a2})₂, wherein each R^{a2} is as defined in any of the embodiments described herein. In some embodiments, each R^{a2} is independently selected from the group consisting of H, —CF₃, and —C₁-C₆ alkyl (e.g., -Me, -Et, -Pr, -ⁱPr, -ⁿBu, -^{t}Bu, -sec-Bu, -iso-Bu).

In some embodiments, each R² is independently selected from the group consisting of halo (e.g., —C₁), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂) and —OCH₃.

In some embodiments, each R² is independently selected from the group consisting of halo (e.g., —C₁), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂), —C₃-C₉ cycloalkyl (e.g., cyclopropyl, cyclobutyl), —C₁-C₆ haloalkoxy, (e.g., —OCF₃, —OCHF₂), —OCH₃, —C(═O) H, —C(═O)NHOH, and —C(═O)NH₂.

In some embodiments, each R² is independently selected from the group consisting of halo (e.g., —C₁), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂), —C₃-C₉ cycloalkyl (e.g., cyclopropyl, cyclobutyl), and —OCH₃.

In some embodiments, each R² is independently selected from the group consisting of halo (e.g., —C₁), —C₁-C₆ alkyl (e.g., -Me, -Et, -Pr, -ⁱPr, -ⁿBu, -^{t}Bu, -sec-Bu, -iso-Bu) and —OCH₃.

In some embodiments, each R² is independently selected from the group consisting of —F, -Me, —CF₃ and —OCH₃.

In some embodiments, each R² is independently selected from the group consisting of -Me, -Et, -^{t}Bu, —CF₃, cyclobutyl and —OCH₃.

In some embodiments, R² is -D.

In certain embodiments, R² is ═O.

In certain embodiments, R² is halo (e.g., fluoro, chloro, bromo, iodo). In some embodiments, R² is —Cl. In some embodiments, R² is —F. In some embodiments, R² is —Br. In some embodiments, R² is —I.

In some embodiments, R² is —CN.

In certain embodiments, R² is —C₁-C₆ alkyl. In some embodiments, R² is -Me. In some embodiments, R² is -Et. In some embodiments R² is -Pr or -iPr.

In some embodiments, R² is —C₁-C₆ heteroalkyl. In some embodiments, R² is methoxymethyl (—CH₂OCH₃). In some embodiments, R² is hydroxymethyl (—CH₂OH). In some embodiments, R² is aminomethyl (e.g., —CH₂NH₂, —CH₂NHCH₃, —CH₂N(CH₃)₂. In some embodiments, R² is —C₁-C₆ haloalkyl. In some embodiments, R² is trifluoromethyl (—CF₃).

In some embodiments, R² is C₃-C₉ cycloalkyl (e.g., cyclopropyl, cyclobutyl, cyclopentyl, cyclohexyl). In some embodiments, R² is cyclopropyl. In some embodiments R² is cyclobutyl. In some embodiments, R² is cyclopentyl. In some embodiments, R² is cyclohexyl.

In some embodiments, R² is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R² is 3-6 membered heterocyclyl (e.g., oxetanyl, tetrahydropyranyl, tetrahydrofuranyl, azetidinyl, pyrrolidinyl, piperidinyl, piperazinyl, morpholinyl). In some embodiments, R² is oxetanyl. In some embodiments, R² is tetrahydropyranyl. In some embodiments, R² is tetrahydrofuranyl. In some embodiments, R² is azetidinyl. In some embodiments, R² is pyrrolidinyl. In some embodiments, R² is piperidinyl. In some embodiments, R² is piperazinyl. In some embodiments, R² is morpholinyl. In some embodiments, R² is azepanyl.

In some embodiments R² is cycloalkylalkyl (e.g., cyclopropylmethyl, cyclobutylmethyl, cyclopentylmethyl, cyclohexylmethyl, cycloheptylmethyl). In some embodiments, R² is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl). In some embodiments, R² is arylalkyl (e.g., benzyl). In some embodiments, R² is heteroarylalkyl (e.g., pyridinylmethyl, thiazolylmethyl, triazolylmethyl, pyrazolylmethyl).

In some embodiments, R² is —CH₂C(═O)N(R^{a2})₂ (e.g., —CH₂C(═O)N(R^{a2})₂).

In some embodiments, R² is —OR^{a2} wherein R^{a2} is as defined in any of the embodiments described herein (e.g., —OH, methoxy, isopropoxy, difluoromethoxy (—OCHF₂), trifluoromethoxy (—OCF₃), ethoxy, propoxy, isopropoxy, cyclopropyloxy, cyclobutyloxy). In some embodiments R² is —OH. In some embodiments, R² is methoxy. In some embodiments, R² is ethoxy. In some embodiments, R² is propoxy. In some embodiments, R² is isopropoxy. In some embodiments, R² is —C₁-C₆ haloalkoxy, (e.g., —OCF₃, —OCHF₂). In some embodiments R² is difluoromethoxy. (—OCHF₂). In some embodiments, R² is trifluoromethoxy (—OCF₃).

In some embodiments, R² is —N(R^{a2})₂ wherein R^{a2} is as defined in any of the embodiments described herein (e.g., —NH₂, —NHR^{a2}, —N(CH₃)R^{a2}). In some embodiments, R² is —NH₂. In some embodiments, R² is —NHR^{a2} (e.g., —NHCH₃, —NHCH₂CH₃, —NHPr, —NHⁱPr, —NHcyclopropyl, —NHcyclobutyl). In some embodiments, R² is —N(CH₃)R^{a2} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R² is —C(═O)R^{a2} wherein R^{a2} is as described herein. In some embodiments R² is —C(═O)R^{a2} wherein R^{a2} is C₁-C₆ alkyl, C₃-C₉ cycloalkyl or 3-10 membered heterocyclyl (e.g., —C(═O)CH₃, —C(═O)CH₂CH₃, —C(═O)^{t}Bu, —C(═O)ⁱPr, —C(═O)CH₂CH₂CH₃, —C(═O)cyclopropyl, —C(═O)cyclobutyl, —C(═O) oxetanyl, —C(═O)tetrahydropyranyl). In some embodiments, R² is —C(═O)CH₃, —C(═O)CH₂CH₃, —C(═O)^{t}Bu, —C(═O)ⁱPr or —C(═O)CH₂CH₂CH₃. In some embodiments, R² is —C(═O)ⁱPr.

In some embodiments, R² is —C(═O)OR^{a2} wherein R^{a2} is as described herein. In some embodiments R² is —C(═O)OR^{a2} wherein R^{a2} is C₁-C₆ alkyl or C₃-C₉ cycloalkyl (e.g., —C(═O)OCH₃, —C(═O)OCH₂CH₃, —C(═O)O^{t}Bu, —C(═O)OⁱPr, —C(═O)OCH₂CH₂CH₃, —C(═O)Ocyclopropyl, —C(═O)Ocyclobutyl). In some embodiments, R² is —C(═O)OCH₃, —C(═O)OCH₂CH₃, —C(═O)O^{t}Bu, —C(═O)ⁱPr or —C(═O)OCH₂CH₂CH₃. In some embodiments, R² is —C(═O)OⁱPr.

In some embodiments, R² is —NR^{a2}C(═O)R^{a2} wherein R^{a2} is as described herein. In certain embodiments, R² is —NHC(═O)R^{a2} (e.g., —NHC(═O)CH₃, —NHC(═O)CH₂CH₃, —NHC(═O)CH₂CH₂CH₃, —NHC(═O)ⁱPr, —NHC(═O)^{t}Bu, —NHC(═O)^{t}Bu, —NHC(═O)Cyclopropyl, —NHC(═O)Cyclobutyl). In certain embodiments, R² is NHC(═O)CH₃. In some embodiments, R² is —N(CH₃)C(═O)R^{a2} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R² is —NR^{a2}C(═O)OR^{a2} wherein R^{a2} is as described herein. In certain embodiments, R² is —NHC(═O)OR^{a2} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R² is —N(CH₃)C(═O)OR^{a2} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R² is —C(═O)N(R^{a2})₂ wherein R^{a2} is as described herein (e.g., —C(═O)NH₂, —C(═O)NHR^{a2}, —C(═O)N(CH₃)R^{a2}). In some embodiments, R² is —C(═O)NH₂. In certain embodiments, R² is —C(═O)NHR^{a2} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R² is —C(═O)N(CH₃)R^{a2} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R² is —OC(═O)N(R^{a2})₂ wherein R^{a2} is as described herein. In certain embodiments, R² is —OC(═O)NHR^{a2} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R² is —OC(═O)N(CH₃)R^{a2} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R² is —S(═O)R^{a2} wherein R^{a2} is as described herein. In certain embodiments, R² is —S(═O)alkyl (e.g., —S(═O)CH₃, —S(═O)CH₂CH₃, —S(═O)CH₂CH₂CH₃, —S(═O)ⁱPr). In certain embodiments, R² is —S(═O)cycloalkyl (e.g., —S(═O)cyclopropyl, —S(═O)cyclobutyl, —S(═O)cyclopentyl, —S(═O)cyclohexyl).

In some embodiments, R² is —S(═O)₂R^{a2}. In certain embodiments, R² is —S(═O)₂alkyl (e.g., —S(═O)₂CH₃, —S(═O)₂CH₂CH₃, —S(═O)₂Pr, —S(═O)₂ⁱPr). In certain embodiments, R² is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R² is S(═O)₂aryl (e.g., —S(═O)₂phenyl).

In some embodiments, R² is —SR^{a2} wherein R^{a2} is as described herein. In certain embodiments, R² is -Salkyl (e.g., —SCH₃, —SCH₂CH₃, —SPr, —SⁱPr). In certain embodiments, R² is -Scycloalkyl (e.g., -Scyclopropyl, —Scyclobutyl, —Scyclopentyl, —Scyclohexyl). In certain embodiments, R² is -Saryl (e.g., -Sphenyl).

In some embodiments, R² is —S(═O)(═NR^{a2})R^{a2} wherein R^{a2} is as described herein. In certain embodiments, R² is —S(═O)(═NH)R^{a2} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)^{t}Bu, —S(═O)(═NH)^{t}Bu, —S(═O)(═NH). Cyclopropyl, —S(═O)(═NH)Cyclobutyl). In some embodiments, R² is —S(═O)(═NCH₃)R^{a2} (e.g., —S(═O)(═NCH₃)CH₃, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R² is —NR^{a2}S(═O)₂R^{a2} wherein R^{a2} is as described herein. In certain embodiments, R² is —NHS(═O)₂alkyl (e.g., —NHS(═O)₂CH₃, —NHS(═O)₂CH₂CH₃, —NHS(═O)₂Pr, —NHS(═O)₂ⁱPr). In certain embodiments, R² is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R² is —N(CH₃) S(═O)₂alkyl (e.g., —N(CH₃) S(═O)₂CH₃, —N(CH₃) S(═O)₂CH₂CH₃, —N(CH₃) S(═O)₂Pr, —N(CH₃) S(═O)₂ⁱPr). In certain embodiments, R² is —N(CH₃) S(═O)₂cycloalkyl (e.g., —N(CH₃) S(═O)₂cyclopropyl, —N(CH₃) S(═O)₂cyclobutyl, —N(CH₃) S(═O)₂cyclopentyl, —N(CH₃) S(═O)₂cyclohexyl).

In some embodiments, R² is —S(═O)₂N(R^{a2})₂ wherein R^{a2} is as described herein. (e.g., —S(═O)₂NH₂, —S(═O)₂NHR^{a2}, —S(═O)₂N(CH₃)R^{a2}). In some embodiments, R² is —S(═O)₂NH₂. In some embodiments, R² is —S(═O)₂NHR^{a2} (e.g., —S(═O)₂NHCH₃, —S(═O)₂NHCH₂CH₃, —S(═O)₂NHPr, —S(═O)₂NHⁱPr, —S(═O)₂NHcyclopropyl, —S(═O)₂NHcyclobutyl). In some embodiments, R² is —S(═O)₂N(CH₃)R^{a2} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R³ is independently selected from the group consisting of -D, —O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein R^{a3} is as described herein, and wherein each alkyl, cycloalkyl, heteroalkyl, heterocyclyl, aryl, heteroaryl, cycloalkylalkyl, heterocyclylalkyl, arylalkyl and heteroarylalkyl of R³ is optionally substituted at any available position (e.g., substituted with 0, 1, 2, 3, 4, or 5 instances of R⁶, wherein each R⁶ is as described herein.

In some embodiments, each alkyl, cycloalkyl, heterocyclyl, aryl, heteroaryl, cycloalkylalkyl, heterocyclylalkyl, arylalkyl and heteroarylalkyl of R³ is unsubstituted. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 1 instance of R⁶. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 2 instances of R⁶. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 3 instances of R⁶. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 4 instances of R⁶. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 5 instances of R⁶.

In some embodiments, each R³ is independently selected from the group consisting of -D, ═O, halo (e.g., —F, —Cl, Br), —CN, —C₁-C₆ alkyl (e.g., -Me, -Et, -Pr, -ⁱPr, -sec-Bu, -^{t}Bu, —CH₂ (CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂, —CH₂OH, —CH(OH)(CH₃), —C(OH)(CH₃)₂, —CH₂NH₂), —C₁-C₆ haloalkyl (e.g., —CHF₂, —CH₂CF₃, —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, piperidin-2-onyl, tetrahydropyridinyl (e.g., 1,2,3,6 tetrahydropyridinyl), piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-pyrrolidinyl, —CH(CH₃)CH₂-pyrrolidinyl, —(CH₂)₂-pyrrolidinyl, —(CH₂)₃-pyrrolidinyl, —CH₂-morpholinyl, —(CH₂)₂-morpholinyl), —OR^{a3} (e.g., —OH, —OCH₃, —OCH₂CH₃, —OCH₂CH₂N(CH₃)₂, —O-tetrahydrofuranyl, —O-tetrahydropyran-4-yl, —OCF₃, —OCHF₂, —OCH₂CH(CH₃)N(CH₃)₂, —Opiperidinyl, —O—(CH₂)₂-pyrrolidinyl, —O—CH₂-piperidinyl, —O—CH₂-oxetanyl, —O—CH₂-tetrahydrofuranyl, —O—CH₂-tetrahydropyranyl), —N(R^{a3})₂, (e.g., —NH₂, —NHR^{a3}, —NHCH₃, —N(CH₃)₂, —NHCH₂CF₃, —NH-oxetan-3-yl, —NH—(N-Me-2-oxo-pyrrolidin-3-yl) and —C(═O)N(R^{a3})₂, (e.g., —C(═O)NH₂, —C(═O)NHCH₃, —C(═O)CH₂CH₂N(CH₃)₂), wherein each alkyl, cycloalkyl, heterocyclyl, cycloalkylalkyl, heterocyclylalkyl, arylalkyl and heteroarylalkyl is optionally substituted (e.g., with 0, 1, 2, 3, 4, or 5 instances of R⁶, and wherein each R^{a3} is as described herein.

In some embodiments, each R³ is independently selected from the group consisting of D, —CN, halo (e.g., —F, —Cl, Br), —C₁-C₆ alkyl (e.g., -Me, -Et, -Pr, -ⁱPr, -sec-Bu, -^{t}Bu, —CH₂ (CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂)—C₁-C₆ haloalkyl, (e.g., —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OR^{a3} (e.g., —OH, —OCH₃, —OCH₂CH₃, —OCH₂CH₂N(CH₃)₂, —OCH₂CH(CH₃)N(CH₃)₂, —OCF₃, —OCHF₂, —O-piperidinyl, —OCH₂-pyrrolidinyl), —NR^{a3})₂, —NR^{a3}C(═O)R^{a3} (e.g., —NHC(═O)CH₃), —NHC(═O)CH₂CH₂N(CH₃)₂), wherein each alkyl, cycloalkyl, heterocyclyl, cycloalkylalkyl, and heterocyclylalkyl is optionally substituted (e.g., with 0, 1, 2, 3, 4, or 5 instances of R⁶; and
each R^{a3} is independently selected from the group consisting of H, —C₁-C₆ alkyl, (e.g., -Me, -Et, -Pr, -ⁱPr, -sec-Bu, -^{t}Bu), —C₁-C₆ haloalkyl (e.g., —CF₃, —CHF₂, —CH₂CF₃), —C₁-C₆ heteroalkyl substituted with 0 or 1 instances of ═O (e.g., —CH₂CH₂N(CH₃)₂, —CH₂CH(CH₃)N(CH₃)₂, —CH₂C(═O)N(CH₃)₂, —CH(CH₃)CH₂N(CH₃)₂, CH(CH₃)C(═O)N(CH₃)₂)), C₃-C₉ cycloalkyl (e.g., cyclopropyl, cyclobutyl, cyclopentyl, cyclohexyl), heterocyclylalkyl (e.g., —CH₂ (N-methyl pyrrolidin-2-yl)) and 3-10 membered heterocyclyl (e.g., piperidinyl, e.g., N-methyl piperidin-4-yl), wherein each R⁶ is as define in any of the embodiments described herein.

In some embodiments, each R³ is independently selected from the group consisting of —CN, halo (e.g., —F, —Cl, Br), —C₁-C₆ alkyl (e.g., -Me, -Et, -Pr, -ⁱPr, -sec-Bu, -^{t}Bu, —CH₂ (CH₃)(Pr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂)—C₁-C₆ haloalkyl, (e.g., —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OR^{a3} (e.g., —OH, —OCH₃, —OCH₂CH₃, —OCH₂CH₂N(CH₃)₂, —OCH₂CH(CH₃)N(CH₃)₂, —OCF₃, —OCHF₂, —O-piperidinyl, —OCH₂-pyrrolidinyl), —NR^{a3}C(═O)R^{a3} (e.g., —NHC(═O)CH₃), —NHC(═O)CH₂CH₂N(CH₃)₂), wherein each alkyl, cycloalkyl, heterocyclyl, cycloalkylalkyl, and heterocyclylalkyl is optionally substituted (e.g., with 0, 1, 2, 3, 4, or 5 instances of R⁶, wherein R⁶ is as described herein).

In some embodiments, R³ is independently selected from the group consisting of -Me, —CN, —F, —Cl, —Br, —CF₃, -Et, -Pr, -ⁱPr, -sec-Bu, -^{t}Bu, —CH₂ (CH₃)(Pr), —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂CH(CH₃)N(CH₃)₂, —CH(CH₃)N(CH₃)₂, —CH₂C(CH₃)₂N(CH₃)₂, cyclopropyl, cyclobutyl, cyclopentyl, cyclohexyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl)piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl, —CH₂-cyclopropyl, —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OH, —OCH₃, —OCH₂CH₃, —OCH₂CH₂N(CH₃)₂, —OCF₃, —OCH₂CH(CH₃)N(CH₃)₂, —OCH₂ (pyrrolidinyl), —Opiperidinyl, —OCHF₂, —C(═O)CH₃), and —C(═O)CH₂CH₂N(CH₃)₂), each optionally substituted (e.g., with 0, 1, 2, 3, 4, or 5 instances of R⁶, wherein R⁶ is as described herein).

In some embodiments, each R³ is independently selected from the group consisting of D, —CN, -Me, -Et, ⁱPr, —F, —Cl, —CF₃, —N(CH₃)₂, —N(CH₂CH₃)₂, —N(CH₃)CH₂CH₃, —OMe, —OEt, —O-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, cyclohexyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 2-azabicyclo[2.2.1]heptanyl, 1-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —OCH₂CH₂N(CH₃)₂, —OCH₂CH(CH₃)N(CH₃)₂, —OCH₂ (pyrrolidinyl), —Opiperidinyl, and —NHC(═O)CH₂CH₂N(CH₃)₂), wherein each cyclopropyl, cyclobutyl, cyclohexyl, tetrahydrofuranyl, tetrahydropyranyl, oxetanyl, morpholinyl, pyrrolidinyl, piperidinyl, tetrahydropyridinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, 2-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —CH(CH₃)CH₂-pyrrolidinyl and —(CH₂)₃-pyrrolidinyl is optionally substituted (e.g., with 0, 1, 2, 3, 4, or 5 instances of R⁶, wherein each R⁶ is as defined in any of the embodiments described herein).

In some embodiments, each R³ is independently selected from the group consisting of —CN, -Me, -Et, -Pr, —F, —Cl, —CF₃, —N(CH₃)₂, —N(CH₃)CH₂CH₃, —OMe, —OEt, —OCH₂-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —OCH₂CH₂N(CH₃)₂, —OCH₂CH(CH₃)N(CH₃)₂, —OCH₂ (pyrrolidinyl), —Opiperidinyl, and —NHC(═O)CH₂CH₂N(CH₃)₂), wherein each cyclopropyl, cyclobutyl, tetrahydropyranyl, oxetanyl, morpholinyl, pyrrolidinyl, piperidinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —(CH₂)₂-pyrrolidinyl, —CH(CH₃)CH₂-pyrrolidinyl and —(CH₂)₃-pyrrolidinyl is optionally substituted (e.g., with 0, 1, 2, 3, 4, or 5 instances of R⁶).

In some embodiments, each R³ is independently selected from the group consisting of —CN, -Me, —F, —Cl, —CF₃, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —OCH₂CH₂N(CH₃)₂, —OCH₂CH(CH₃)N(CH₃)₂, —OCH₂ (pyrrolidinyl), —Opiperidinyl, and —NHC(═O)CH₂CH₂N(CH₃)₂), wherein each cyclopropyl, cyclobutyl, tetrahydropyranyl, oxetanyl, morpholinyl, pyrrolidinyl, piperidinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —(CH₂)₂-pyrrolidinyl, —CH(CH₃)CH₂-pyrrolidinyl and —(CH₂)₃-pyrrolidinyl is optionally substituted (e.g., with 0, 1, 2, 3, 4, or 5 instances of R⁶).

In some embodiments, each R³ is independently selected from the group consisting of: D, —CN, -Me, -Et, -ⁱPr, —F, —Cl, —CF₃, —N(CH₃)₂, —N(CH₃)CH₂CH₃, —OMe, —OEt, —OCH₂-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, cyclohexyl, tetrahydrofuran-3-yl, tetrahydropyran-4-yl, oxetan-3-yl, morpholin-2-yl, piperazin-4-yl, pyrrolidin-1-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 4-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl, —O(CH₂)₂N(CH₃)₂—OCH₂ (pyrrolidin-2-yl), —Opiperidin-4-yl, —OCH₂CH(CH₃)N(CH₃)₂ and —NHC(═O)(CH₂)₂N(CH₃)₂, wherein each cyclopropyl, cyclobutyl, cyclohexyl, tetrahydrofuran-3-yl, tetrahydropyran-4-yl, oxetan-3-yl, morpholin-2-yl, piperazin-4-yl, pyrrolidin-1-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 4-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl and —CH(CH₃)CH₂-pyrrolidin-1-yl is optionally substituted (e.g., with 0, 1, 2, 3, 4, or 5 instances of R⁶, wherein each R⁶ is as described in any of the embodiments described herein.

In some embodiments, each R³ is independently selected from the group consisting of: —CN, -Me, -Et, -Pr, —F, —Cl, —CF₃, —N(CH₃)₂, —N(CH₃)CH₂CH₃, —OMe, —OEt, —OCH₂-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuran-3-yl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl, —O(CH₂)₂N(CH₃)₂—OCH₂ (pyrrolidin-2-yl), —Opiperidin-4-yl, —OCH₂CH(CH₃)N(CH₃)₂ and —NHC(═O)(CH₂)₂N(CH₃)₂, wherein each cyclopropyl, cyclobutyl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, piperazin-4-yl, azetidin-3-yl, 2-azabicyclo[2.2.2]octan-4-yl, 2-azaspiro[3.3]heptan-6-yl, 1-azabicyclo[2.2.1]heptan-4-yl, —CH₂-piperidin-4-yl, —CH₂-pyrrolidin-1-yl, —(CH₂)₂-pyrrolidin-1-yl and —CH(CH₃)CH₂-pyrrolidin-1-yl is optionally substituted (e.g., with 0, 1, 2, 3, 4, or 5 instances of R⁶).

In some embodiments, each R³ is independently selected from the group consisting of: —CN, -Me, —F, —Cl, —CF₃, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuran-3-yl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl, —O(CH₂)₂N(CH₃)₂—OCH₂ (pyrrolidin-2-yl), —Opiperidin-4-yl, —OCH₂CH(CH₃)N(CH₃)₂ and —NHC(═O)(CH₂)₂N(CH₃)₂, wherein each cyclopropyl, cyclobutyl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, piperazin-4-yl, azetidin-3-yl, 2-azabicyclo[2.2.2]octan-4-yl, 2-azaspiro[3.3]heptan-6-yl, 1-azabicyclo[2.2.1]heptan-4-yl, —CH₂-piperidin-4-yl, —CH₂-pyrrolidin-1-yl, —(CH₂)₂-pyrrolidin-1-yl and —CH(CH₃)CH₂-pyrrolidin-1-yl is optionally substituted (e.g., with 0, 1, 2, 3, 4, or 5 instances of R⁶).

In some embodiments, R³ is selected from the group consisting of -D, —CN, -Me, -Et, ⁱPr, —OMe, —OEt, —F, —Cl, —CF₃,

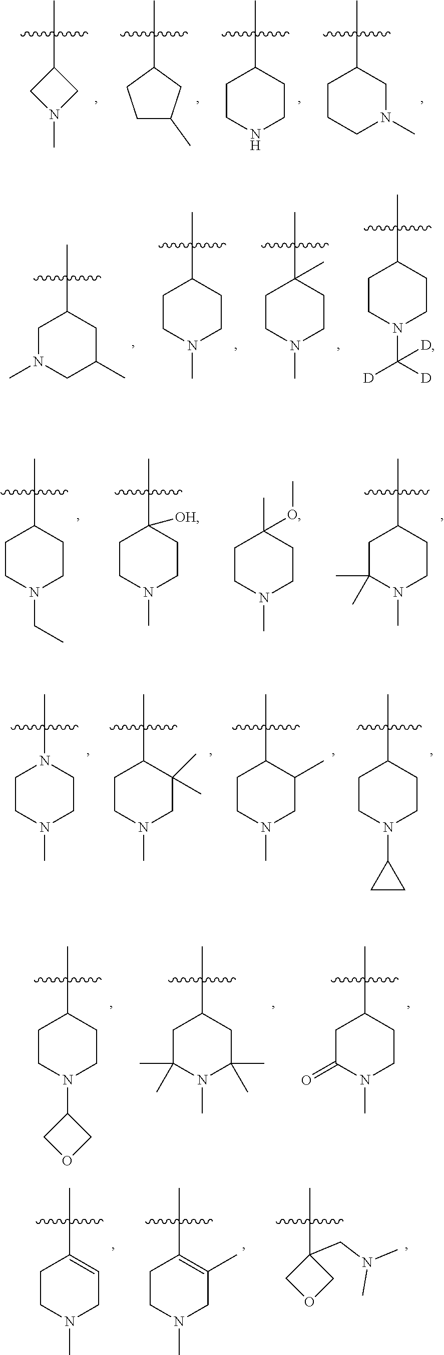

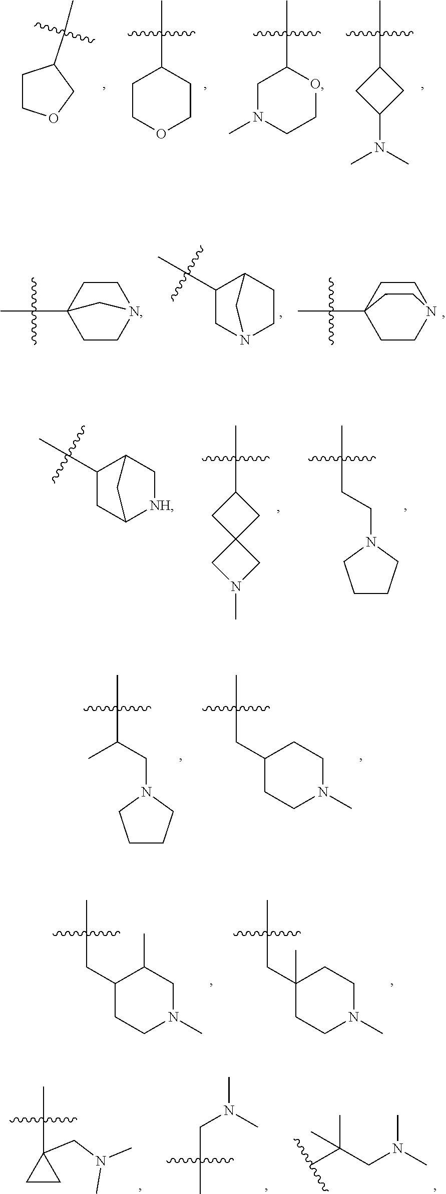

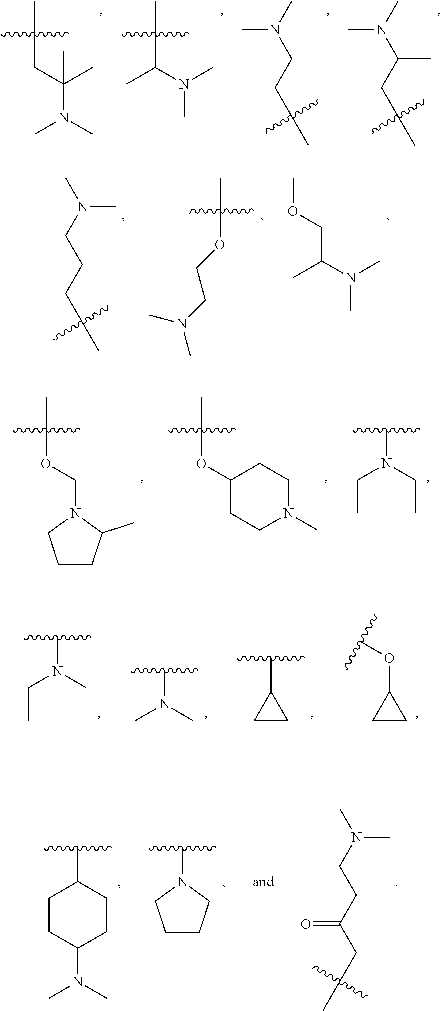

In some embodiments, R³ is selected from the group consisting of -D, —CN, -Me, -Et, -ⁱPr, —OMe, —OEt, —F, —Cl, —CF₃,

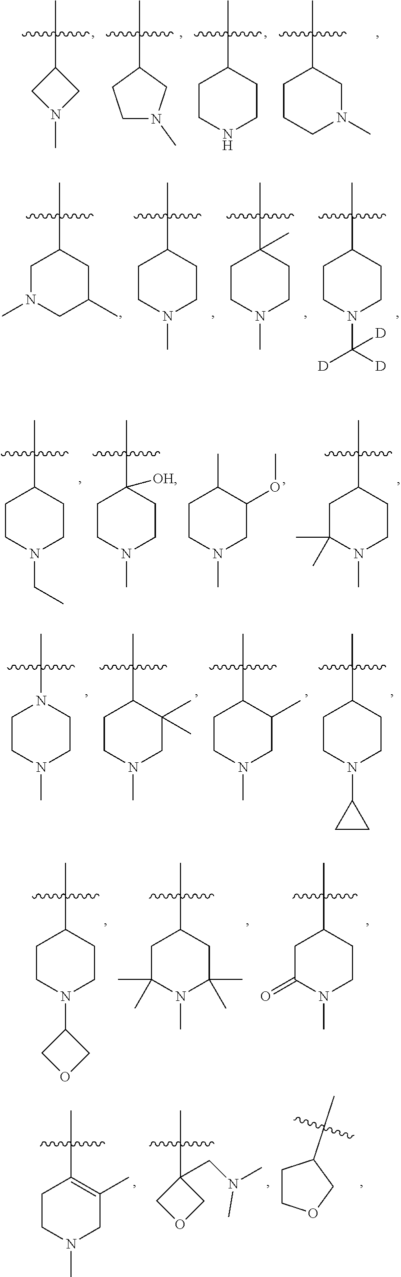

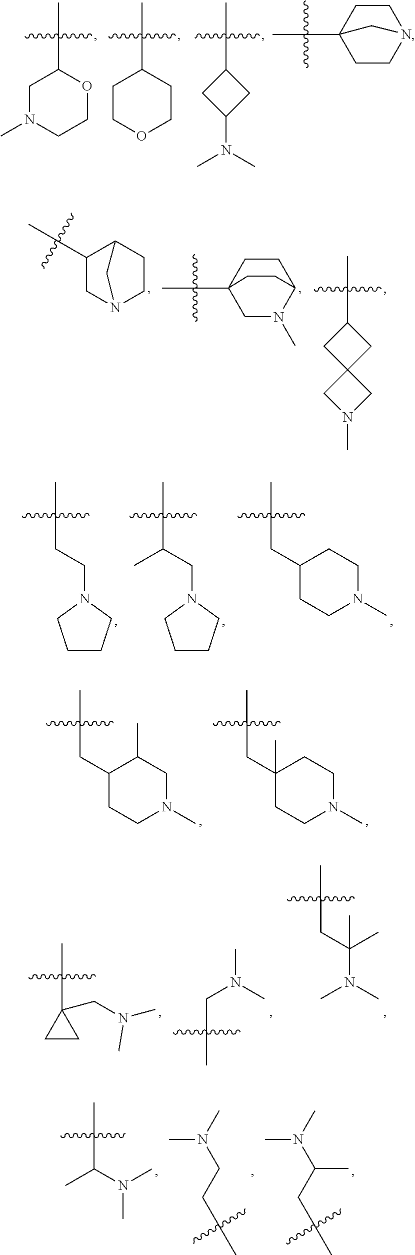

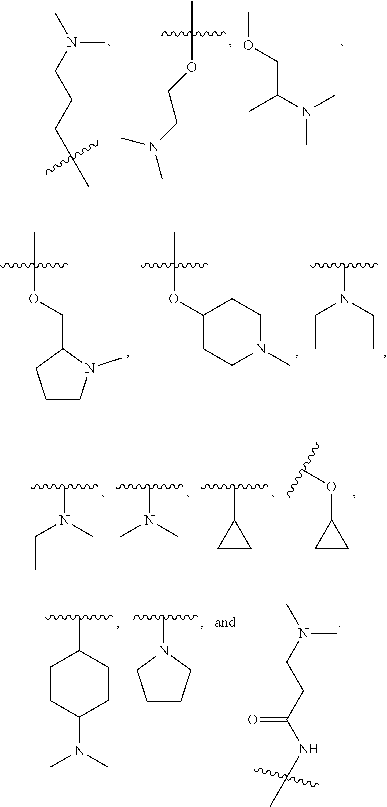

In some embodiments, R³ is selected from the group consisting of -D, —CN, -Me, —F, —Cl, —CF₃,

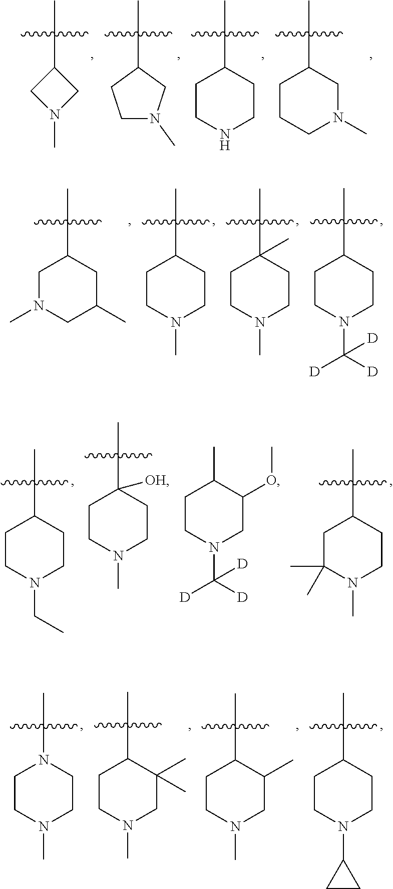

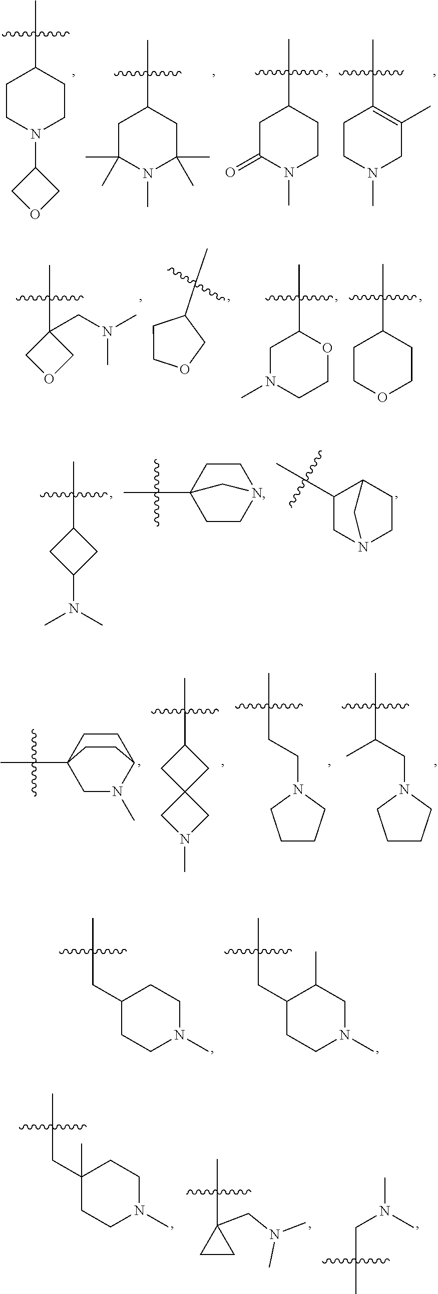

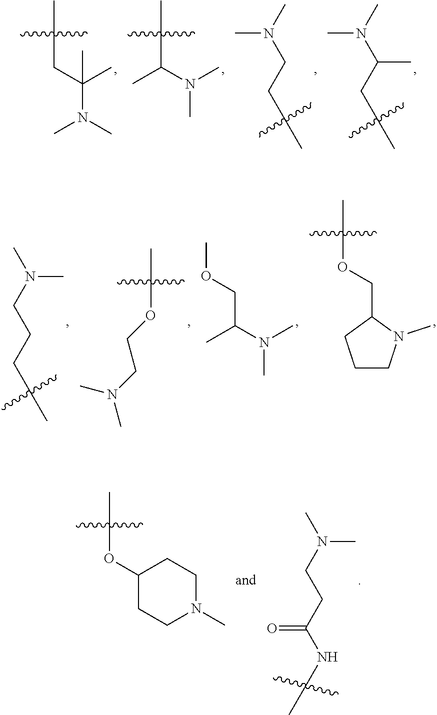

In some embodiments R³ is -D.

In certain embodiments, R³ is —C₁-C₆ alkyl. In some embodiments, R³ is -Me. In some embodiments, R³ is -Et. In some embodiments R³ is -Pr or -ⁱPr. In some embodiments R³ is -^{t}Bu or -sec-Bu. In some embodiments R³ is —CH₂ (CH₃)(Pr).

In certain embodiments, R³ is halo (e.g., fluoro, chloro, bromo, iodo). In some embodiments, R³ is —Cl. In some embodiments, R³ is —F. In some embodiments, R³ is —Br. In some embodiments, R³ is —I.

In some embodiments, R³ is —CN.

In some embodiments, R³ is —C₁-C₆ heteroalkyl. In some embodiments, R³ is methoxymethyl (—CH₂OCH₃). In some embodiments, R³ is hydroxymethyl (—CH₂OH). In some embodiments, R³ is —CH(OH)CH₃, —C(OH)(CH₃)₂. In some embodiments, R³ is aminomethyl (e.g., —CH₂NH₂, —CH₂NHCH₃, —CH₂NHCH₂CH₃—CH₂N(CH₃)₂, —CH(CH₃)(N(CH₃)₂), —CH(CH₃)CH₂ (N(CH₃)₂), —CH₂CH₂N(CH₃)₂, —CH₂CH₂CH₂N(CH₃)₂, —CH₂CH₂N(Me)(oxetan-3-yl), —CH(CH₃)N(CH₃)₂, —CH₂C(CH₃)₂N(CH₃)₂, —CH₂CH(CH₃)(N(CH₃)₂). In some embodiments, the heteroalkyl is further substituted with ═O (e.g., —CH₂NHC(═O)CH₃). In some embodiments, R³ is —C₁-C₆ haloalkyl. In some embodiments, R³ is trifluoromethyl (—CF₃). In other embodiments, R³ is difluoromethyl (—CHF₂). In some embodiments R³ is trifluoroethyl (—CH₂CF₃).

In some embodiments, R³ is C₃-C₉ cycloalkyl (e.g., cyclopropyl, cyclobutyl, cyclopentyl, cyclohexyl). In some embodiments, R³ is cyclopropyl. In some embodiments, the cyclopropyl is substituted with 1 instance of R⁶. In some embodiments, R³ is cycloprop-1-yl substituted at the 1 position with R⁶. In some embodiments R³ is cyclobutyl. In some embodiments, R³ is cyclopentyl. In some embodiments, R³ is cyclohexyl. In some embodiments, the cycloalkyl is substituted with —OH (e.g., hydroxycyclobutyl). In some embodiments, R³ is

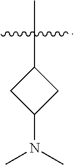

In some embodiments, R³ is

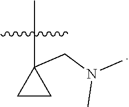 [SMILES: *C1(CN(C)C)CC1]

In some embodiments, R³ is

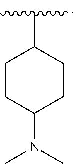 [SMILES: *C1CCC(N(C)C)CC1]

In some embodiments, R³ is 3-10 membered heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2, 3, 4 or 5 instances of R⁶.

In some embodiments, R³ is 3-8 membered monocyclic heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl) or 5-10 membered bicyclic heterocyclyl (decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl) each substituted with 0, 1, 2, 3, 4 or 5 instances of R⁶.

In some embodiments, R³ is 3-8 membered monocyclic heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl), each substituted with 0, 1, 2, 3, 4 or 5 instances of R⁶. In some embodiments, R³ is selected from the group consisting of oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl and piperazin-2-onyl, each substituted with 0, 1, 2, 3, 4 or 5 instances of R⁶. In some embodiments, R³ is oxetanyl. In some embodiments, R³ is tetrahydropyranyl. In some embodiments, R³ is tetrahydrofuranyl. In some embodiments, R³ is azetidinyl. In some embodiments, R³ is pyrrolidinyl. In some embodiments, R³ is piperidinyl. In some embodiments, R³ is tetrahydropyridinyl (e.g., 1,2,3,6 tetrahydropyridinyl). In some embodiments, R³ is piperazinyl. In some embodiments, R³ is morpholinyl. In some embodiments, R³ is azepanyl. In some embodiments, R³ is piperidin-2-onyl. In some embodiments, R³ is piperazin-2-onyl.

In some embodiments, R³ is selected from the group consisting of azetidin-3-yl, tetrahydrofuran-3-yl, tetrahydropyran-4-yl, oxetan-3-yl, morpholin-2-yl, pyrrolidin-1-yl, pyrrolidin-3-yl, piperidin-4-yl, 1,2,3,6 tetrahydropyridin-4-yl, piperidin-3-yl, piperidin-2-one-4-yl, piperazin-4-yl and piperazin-2-on-5-yl. In some embodiments, R³ is selected from the group consisting of azetidin-3-yl, tetrahydropyran-4-yl, oxetan-3-yl, morpholin-2-yl, pyrrolidin-3-yl, piperidin-4-yl, piperidin-3-yl, 1,2,3,6 tetrahydropyridin-4-yl, piperidin-2-one-4-yl and piperazin-4-yl.

In some embodiments, R³ is tetrahydrofuran-3-yl. In some embodiments, R³ is tetrahydropyran-4-yl. In some embodiments, R³ is oxetan-3-yl. In some embodiments, R³ is morpholin-2-yl. In some embodiments, R³ is pyrrolidin-1-yl. In some embodiments, R³ is pyrrolidin-3-yl. In some embodiments, R³ is piperidin-4-yl. In some embodiments, R³ is piperidin-3-yl. In some embodiments, R³ is 1,2,3,6 tetrahydropyridin-4-yl. In some embodiments, R³ is piperidin-2-one-4-yl. In some embodiments, R³ is piperazin-4-yl. In some embodiments, R³ is piperazin-2-on-5-yl. In some embodiments, R³ is azetidin-3-yl.

In some embodiments, R³ is

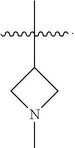 [SMILES: *C1CN(C)C1]

In some embodiments, R³ is

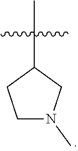 [SMILES: *C1CCN(C)C1]

In some embodiments, R³ is

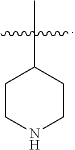 [SMILES: *C1CCNCC1]

In some embodiments, R³ is

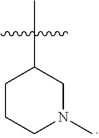 [SMILES: *C1CCCN(C)C1]

In some embodiments, R³ is

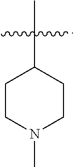 [SMILES: *C1CCN(C)CC1]

In some embodiments, R³ is

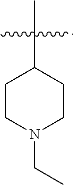 [SMILES: *C1CCN(CC)CC1]

In some embodiments, R³ is

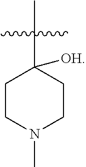 [SMILES: *C1(O)CCN(C)CC1]

In some embodiments, R³ is

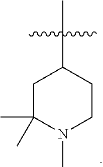 [SMILES: *C1CCN(C)C(C)(C)C1]

In some embodiments, R³ is

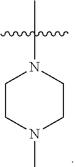 [SMILES: *N1CCN(C)CC1]

In some embodiments, R³ is

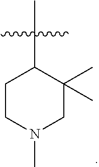 [SMILES: *C1CCN(C)CC1(C)C]

In some embodiments, R³ is

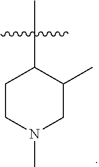 [SMILES: *C1CCN(C)CC1C]

In some embodiments, R³ is

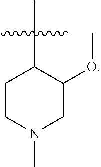 [SMILES: *C1CCN(C)CC1OC]

In some embodiments, R³ is

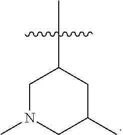

In some embodiments, R³ is

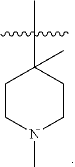 [SMILES: *C1(C)CCN(C)CC1]

In some embodiments, R³ is

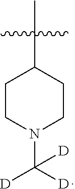 [SMILES: *C1CCN(C([2H])([2H])[2H])CC1]

In some embodiments, R³ is

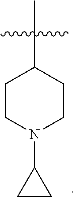 [SMILES: *C1CCN(C2CC2)CC1]

In some embodiments, R³ is

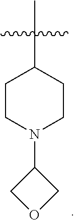 [SMILES: *C1CCN(C2COC2)CC1]

In some embodiments, R³ is

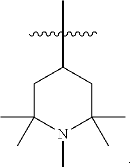

In some embodiments, R³ is

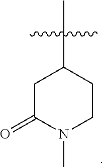 [SMILES: *C1CCN(C)C(=O)C1]

In some embodiments, R³ is

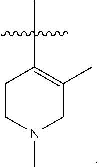 [SMILES: *C1=C(C)CN(C)CC1]

In some embodiments, R³ is

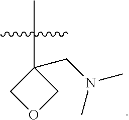 [SMILES: *C1(CN(C)C)COC1]

In some embodiments, R³ is

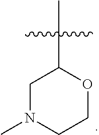 [SMILES: *C1CN(C)CCO1]

In some embodiments, R³ is

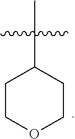 [SMILES: *C1CCOCC1]

In some embodiments, R³ is

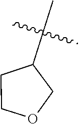 [SMILES: *C1CCOC1]

In some embodiments, R³ is

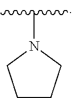 [SMILES: *N1CCCC1]

In some embodiments, R³ is a 5-10 membered bicyclic heterocyclyl (e.g., decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 2-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2, 3, 4 or 5 instances of R⁶.

In some embodiments, R³ is a 5-10 membered bicyclic heterocyclyl (e.g., decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2, 3, 4 or 5 instances of R⁶.

In some embodiments, R³ is selected from the group consisting of decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl and decahydro-2,7-naphthyridinyl, each substituted with 0, 1, 2, 3, 4 or 5 instances of R⁶.

In some embodiments, R³ is selected from the group consisting of 2-azaspiro[3.3]heptanyl, 2-azabicyclo[2.2.2]octanyl, 1-azabicyclo[2.2.1]heptanyl, and 2-azabicyclo[2.2.1]heptanyl each substituted with 0 or 1 instances of R⁶.

In some embodiments, R³ is selected from the group consisting of 2-azaspiro[3.3]heptanyl, 2-azabicyclo[2.2.2]octanyl and 1-azabicyclo[2.2.1]heptanyl.

In some embodiments, R³ is selected from the group consisting of decahydro-1,6-naphthyridin-6-yl, 2-azaspiro[3.3]heptan-6-yl, 5-oxa-2,8-diazaspiro[3.5]nonan-8-yl, 8-azabicyclo[3.2.1]octan-3-yl, 2-azabicyclo[2.2.2]octan-4-yl, 3-azabicyclo[3.2.0]heptan-6-yl, 3-azabicyclo[3.1.1]heptan-1-yl, 3-azabicyclo[3.1.0]hexan-6-yl, 1-azabicyclo[2.2.1]heptan-4-yl, 2-azabicyclo[2.2.1]heptan-4-yl, 3-azabicyclo[3.2.0]heptan-6-yl, 2-azabicyclo[2.1.1]hexan-4-yl, bicyclo[1.1.1]pentan-2-yl, octahydrocyclopenta[c]pyrrol-5-yl, decahydro-1,6-naphthyridin-6-yl, octahydro-1H-pyrrolo[3,4-c]pyridine-5-yl and decahydro-2,7-naphthyridin-2-yl.

In some embodiments, R³ is selected from the group consisting of decahydro-1,6-naphthyridin-6-yl, 2-azaspiro[3.3]heptan-6-yl, 5-oxa-2,8-diazaspiro[3.5]nonan-8-yl, 8-azabicyclo[3.2.1]octan-3-yl, 2-azabicyclo[2.2.2]octan-4-yl, 3-azabicyclo[3.2.0]heptan-6-yl, 3-azabicyclo[3.1.1]heptan-1-yl, 3-azabicyclo[3.1.0]hexan-6-yl, 1-azabicyclo[2.2.1]heptan-4-yl, 3-azabicyclo[3.2.0]heptan-6-yl, 2-azabicyclo[2.1.1]hexan-4-yl, bicyclo[1.1.1]pentan-2-yl, octahydrocyclopenta[c]pyrrol-5-yl, decahydro-1,6-naphthyridin-6-yl, octahydro-1H-pyrrolo[3,4-c]pyridine-5-yl and decahydro-2,7-naphthyridin-2-yl.

In some embodiments, R³ is selected from the group consisting of 2-azabicyclo[2.2.2]octan-4-yl, 2-azaspiro[3.3]heptan-6-yl, 2-azabicyclo[2.2.1]heptan-4-yl and 1-azabicyclo[2.2.1]heptan-4-yl.

In some embodiments, R³ is selected from the group consisting of 2-azabicyclo[2.2.2]octan-4-yl, 2-azaspiro[3.3]heptan-6-yl and 1-azabicyclo[2.2.1]heptan-4-yl.

In some embodiments, R³ is decahydro-1,6-naphthyridinyl. In some embodiments, R³ is 2-azaspiro[3.3]heptanyl. In some embodiments, R³ is 5-oxa-2,8-diazaspiro[3.5]nonanyl. In some embodiments, R³ is 8-azabicyclo[3.2.1]octanyl. In some embodiments, R³ is 2-azabicyclo[2.2.2]octanyl. In some embodiments, R³ is 3-azabicyclo[3.2.0]heptanyl. In some embodiments, R³ is 3-azabicyclo[3.1.1]heptanyl. In some embodiments, R³ is 3-azabicyclo[3.1.0]hexanyl. In some embodiments, R³ is 2-azabicyclo[2.1.1]hexanyl. In some embodiments, R³ is 1-azabicyclo[2.2.1]heptanyl. In some embodiments, R³ is 2-azabicyclo[2.2.1]heptanyl. In some embodiments, R³ is 3-azabicyclo[3.2.0]heptanyl. In some embodiments, R³ is bicyclo[1.1.1]pentanyl. In some embodiments, R³ is octahydrocyclopenta[c]pyrrolyl. In some embodiments, R³ is decahydro-1,6-naphthyridinyl. In some embodiments, R³ is octahydro-1H-pyrrolo[3,4-c]pyridinyl. In some embodiments, R³ is decahydro-2,7-naphthyridinyl.

In some embodiments, R³ is decahydro-1,6-naphthyridin-6-yl. In some embodiments, R³ is 2-azaspiro[3.3]heptan-6-yl. In some embodiments, R³ is 5-oxa-2,8-diazaspiro[3.5]nonan-8-yl. In some embodiments, R³ is 8-azabicyclo[3.2.1]octan-3-yl. In some embodiments, R³ is 2-azabicyclo[2.2.2]octan-4-yl. In some embodiments, R³ is 3-azabicyclo[3.2.0]heptan-6-yl. In some embodiments, R³ is 3-azabicyclo[3.1.1]heptan-1-yl. In some embodiments, R³ is 3-azabicyclo[3.1.0]hexan-6-yl. In some embodiments, R³ is 1-azabicyclo[2.2.1]heptan-4-yl. In some embodiments, R³ is 2-azabicyclo[2.2.1]heptan-4-yl. In some embodiments, R³ is 3-azabicyclo[3.2.0]heptan-6-yl. In some embodiments, R³ is 2-azabicyclo[2.1.1]hexan-4-yl. In some embodiments, R³ is bicyclo[1.1.1]pentan-2-yl. In some embodiments, R³ is octahydrocyclopenta[c]pyrrol-5-yl. In some embodiments, R³ is decahydro-1,6-naphthyridin-6-yl. In some embodiments, R³ is octahydro-1H-pyrrolo[3,4-c]pyridine-5-yl. In some embodiments, R³ is decahydro-2,7-naphthyridin-2-yl.

In some embodiments, R³ is

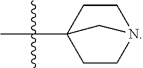 [SMILES: *C12CCN(CC1)C2]

In some embodiments, R³ is

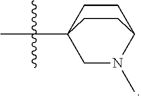 [SMILES: *C12CCC(CC1)N(C)C2]

In some embodiments, R³ is

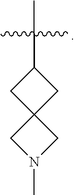 [SMILES: *C1CC2(C1)CN(C)C2]

In some embodiments, R³ is

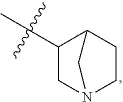 [SMILES: *C1CN2CCC1C2]

In some embodiments, R³ is

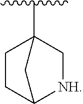 [SMILES: *C12CCC(C1)NC2]

In some embodiments, the heterocyclyl is substituted with 0, 1, 2, 3, 4 or 5 instances of -D, ═O, -Me, -CD₃, -Et, —C(═O)CH₃ cyclopropyl, oxetan-3-yl, —OH, —N(CH₃)₂, —CH₂N(CH₃)₂ or —C(═O)NHCH₃. In some embodiments, the heterocyclyl is substituted with 0, 1, 2, 3, 4 or 5 instances of -Me, -CD₃, -Et, cyclopropyl, oxetan-3-yl, —OH, ═O, —N(CH₃)₂ and —CH₂N(CH₃)₂.

In some embodiments, R³ is monocyclic heterocyclylalkyl (e.g., oxetanylmethyl, aziridinylmethyl, tetrahydrofuranylmethyl, pyrrolidinylmethyl, pyrrolidinylethyl, pyrrolidinylpropyl, —CH(CH₃)CH₂-pyrrolidinyl, tetrahydropyranylmethyl, piperidinylmethyl, piperazinylmethyl, morpholinylmethyl, azepanylmethyl). In some embodiments, R³ is selected from the group consisting of —CH₂-oxetan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl. In some embodiments, R³ is selected from the group consisting of —CH₂-piperidin-4-yl, —CH₂-pyrrolidin-1-yl, —(CH₂)₂-pyrrolidin-1-yl, —CH(CH₃)CH₂-pyrrolidin-1-yl. In some embodiments, R³ is selected from the group consisting of —CH₂-piperidin-4-yl, —CH₂-pyrrolidin-1-yl, —(CH₂)₂-pyrrolidin-1-yl, —CH(CH₃)CH₂-pyrrolidin-1-yl.

In some embodiments, R³ is —CH₂-morpholin-4-yl. In some embodiments, R³ is —(CH₂)₂-morpholin-4-yl. In some embodiments, R³ is —CH₂-oxetan-3-yl. In some embodiments, R³ is —CH₂-piperidin-4-yl. In some embodiments, R³ is —CH₂-pyrrolidin-1-yl. In some embodiments, R³ is —(CH₂)₂-pyrrolidin-1-yl. In some embodiments, R³ is —CH(CH₃)CH₂-pyrrolidin-1-yl. In some embodiments, the monocyclic heterocyclylalkyl is unsubstituted. In some embodiments, the monocyclic heterocyclylalkyl e.g., substituted with 0, 1, 2, 3, 4, or 5 instances of R⁶, wherein R⁶ is as described herein. In some embodiments, the monocyclic heterocyclylalkyl is substituted with 0, 1 or 2 instances of -Me. In some embodiments R³ is cycloalkylalkyl (e.g., cyclopropylmethyl, cyclobutylmethyl, cyclopentylmethyl, cyclohexylmethyl, cycloheptylmethyl). In some embodiments, R³ is cyclopropylmethyl.

In some embodiments, R³ is a 5-10 membered heteroaryl (e.g., a 5-6 membered monocyclic heteroaryl or an 8-10 membered bicyclic heteroaryl containing 1-3 heteroatoms selected from the group consisting of N, O and S). In some embodiments, R³ is a 5-6 membered monocyclic heteroaryl (e.g., a 5-membered monocyclic heteroaryl containing 1-3 heteroatoms selected from the group consisting of O, N and S, a 6-membered monocyclic heteroaryl containing 1-3 N heteroatoms). In some embodiments, R³ is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R³ is thiophenyl (e.g., thiophen-2-yl, thiophen-3-yl). In some embodiments, R³ is pyrazolyl (e.g., pyrazol-1-yl, pyrazol-3-yl, pyrazol-5-yl). In some embodiments, R³ is thiazolyl (e.g., thiazol-2-yl, thiazol-4-yl, thiazol-5-yl). In some embodiments, R³ is a 6-membered monocyclic heteroaryl (e.g., pyridyl, pyrimidinyl, triazinyl, pyrazinyl, pyridazinyl). In some embodiments, R³ is pyridinyl (e.g., pyridin-2-yl, pyridin-3-yl, pyridin-4-yl). In some embodiments, R³ is pyrimidinyl (e.g, pyrimidin-2-yl, pyrimidin-4-yl, pyrimidin-5-yl). In some embodiments, the heteroaryl is substituted with 0, 1, 2, 3, 4 or 5 instances of R⁶ as defined in any of the embodiments described herein.

In some embodiments, R³ is a 6-10 membered mono or bicyclic aryl. In some embodiments, R³ is phenyl. In some embodiments, the phenyl is substituted with 0, 1, 2, 3, 4 or 5 instances of R⁶ as defined in any of the embodiments described herein.

In some embodiments, R³ is arylalkyl. In some embodiments, R³ is benzyl.

In some embodiments, R³ is heteroarylalkyl (e.g., pyridinylmethyl, thiazolylmethyl, triazolylmethyl, pyrazolylmethyl).

In some embodiments, R³ is —OR^{a3} (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy, —O(CH₂)₂N(CH₃)₂, —O-tetrahydrofuran-3-yl, —O-tetrahydropyran-4-yl, —O(N-methyl piperidin-4-yl), —O—(N-Me-2-oxo-pyrrolidin-3-yl), —OCH₂CH(CH₃)N(CH₃)₂, —OCH₂ (N-Methylpyrrolidin-2-yl), —O(N-methyl piperidin-4-yl), —O—(CH₂)₂-pyrrolidin-2-yl, —O—CH₂-piperidin-4-yl, —O—CH₂-oxetan-3-yl). In some embodiments, R³ is hydroxy. In some embodiments, R³ is methoxy. In some embodiments, R³ is difluoromethoxy (—OCHF₂). In some embodiments, R³ is trifluoromethoxy (—OCF₃). In some embodiments, R³ is ethoxy. In some embodiments, R³ is propoxy. In some embodiments, R³ is isopropoxy. In some embodiments, R³ is cyclopropyloxy. In some embodiments, R³ is cyclobutyloxy. In some embodiments, R³ is —O(CH₂)₂N(CH₃)₂. In some embodiments, R³ is —O-tetrahydrofuran-3-yl. In some embodiments, R³ is —O-tetrahydropyran-4-yl. In some embodiments, R³ is —O(N-methyl piperidin-4-yl). In some embodiments, R³ is —OCH₂ (N-Methylpyrrolidin-2-yl). In some embodiments, R³ is —O—(N-Me-2-oxo-pyrrolidin-3-yl). In some embodiments, R³ is —O—(CH₂)₂-pyrrolidin-2-yl. In some embodiments, R³ is —O—CH₂-piperidin-4-yl. In some embodiments, R³ is —O—CH₂-oxetan-3-yl.

In some embodiments, R³ is hydroxy. In some embodiments, R³ is methoxy. In some embodiments, R³ is ethoxy. In some embodiments, R³ is propoxy. In some embodiments, R³ is isopropoxy. In some embodiments R³ is difluoromethoxy. (—OCHF₂). In some embodiments, R³ is trifluoromethoxy (—OCF₃). In some embodiments, R³ is —O(CH₂)₂N(CH₃)₂.

In some embodiments, R³ is —N(R^{a3})₂ wherein R^{a3} is as described herein (e.g., —NH₂, —NHR^{a3}, —N(CH₃)R^{a3}). In some embodiments, R³ is —NH₂. In some embodiments, R³ is —NHR^{a3} (e.g., —NHCH₃, —NHCH₂CH₃, —NHPr, —NHCH₂CF₃, —NHⁱPr, —NHcyclopropyl, —NHcyclobutyl). In some embodiments, R³ is NHCH₂CF₃. In some embodiments, R³ is —N(CH₃)R^{a3} (e.g., —N(CH₃)₂, —N(CH₃)CH₂CH₃, —N(CH₂CH₃)₂, —N(CH₃)CH₂CH₂CH₃, —N(CH₃)ⁱPr, —N(CH₃)cyclopropyl, —N(CH₃)cyclobutyl).

In some embodiments, R³ is —C(═O)R^{a3} or —C(═O)OR^{a3} wherein R^{a3} is as described herein. In some embodiments, R³ is —C(═O)R^{a3} wherein R^{a3} is as described herein. In some embodiments, R³ is —C(═O)alkyl. In some embodiments, R³ is —C(═O)CH₃, —C(═O)cyclopropyl, —C(═O)cyclobutyl, —C(═O)^{t}Bu, —C(═O)ⁱPr, —C(═O)CH₂CH₂CH₃, —C(═O)OCH₃ or —C(O)CH₂CH₂N(CH₃)₂. In some embodiments, R³ is acetyl (—C(═O)CH₃). In some embodiments, R³ is —C(═O)CH₂CH₂N(CH₃)₂. In some embodiments, R³ is —C(═O)OR^{a3}. In some embodiments, R³ is —COOH. In some embodiments, R³ is COOCH₃.

In some embodiments, R³ is —NR^{a3}C(═O)R^{a3} wherein R^{a3} is as described herein. In certain embodiments, R³ is —NHC(═O)R^{a3} (e.g., —NHC(═O)CH₃, —NHC(═O)CH₂CH₃, —NHC(═O)CH₂CH₂CH₃, —NHC(═O) ⁱPr, —NHC(═O)^{t}Bu, —NHC(═O)^{t}Bu, —NHC(═O)Cyclopropyl, —NHC(═O)Cyclobutyl, —C(O)CH₂CH₂N(CH₃)₂.). In some embodiments, R³ is —NHC(═O)CH₃. In some embodiments, R³ is —C(O)CH₂CH₂N(CH₃)₂.

In some embodiments, R³ is —N(CH₃)C(═O)R^{a3} wherein R^{a3} is as described herein (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)^{t}Bu, —N(CH₃)C(═O)^{t}Bu, —N(CH₃)C(═O)Cyclopropyl, —N(CH₃)C(═O)Cyclobutyl).

In some embodiments, R³ is —NR^{a3}C(═O)OR^{a3} wherein R^{a3} is as described herein. In certain embodiments, R³ is —NHC(═O)OR^{a3} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R³ is —N(CH₃)C(═O)OR^{a3} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R³ is —C(═O)N(R^{a3})₂ wherein R^{a3} is as described herein (e.g., —C(═O)NH₂, —C(═O)NHR^{a3}, —C(═O)N(CH₃)R^{a3}). In some embodiments, R³ is —C(═O)NH₂. In certain embodiments, R³ is —C(═O)NHR^{a3} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In some embodiments, R³ is —C(═O)NHCH₃. In certain embodiments, R³ is —C(═O)N(CH₃)R^{a3} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)^{t}Bu, —C(═O)N(CH₃)^{t}Bu, —C(═O)N(CH₃)Cyclopropyl, —C(═O)N(CH₃)Cyclobutyl).

In some embodiments, R³ is —OC(═O)N(R^{a3})₂ wherein R^{a3} is as described herein. In certain embodiments, R³ is —OC(═O)NHR^{a3} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R³ is —OC(═O)N(CH₃)R^{a3} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R³ is —OC(═O)R^{a3} wherein R^{a3} is as described herein. (e.g., —OC(═O)CH₃, —OC(═O)CH₂CH₃, —OC(═O)CH₂CH₂CH₃, —OC(═O) \CH₂CH₂CH₃, —OC(═O)Bu, —OC(═O)Bu, —OC(═O)Cyclopropyl, —OC(═O)Cyclobutyl). In some embodiments, R³ is —OC(═O)CH₃.

In some embodiments, R³ is —S(═O)R^{a3} wherein R^{a3} is as described herein. In certain embodiments, R³ is —S(═O)alkyl (e.g., —S(═O)CH₃, —S(═O)CH₂CH₃, —S(═O)CH₂CH₂CH₃, —S(═O)ⁱPr). In some embodiments R³ is —S(═O)CH₃. In certain embodiments, R³ is —S(═O)cycloalkyl (e.g., —S(═O)cyclopropyl, —S(═O)cyclobutyl, —S(═O)cyclopentyl, —S(═O)cyclohexyl).

In some embodiments, R³ is —S(═O)₂R^{a3} wherein R^{a3} is as described herein. In certain embodiments, R³ is —S(═O)₂alkyl (e.g., —S(═O)₂CH₃, —S(═O)₂CH₂CH₃, —S(═O)₂Pr, —S(═O)₂ⁱPr). In some embodiments R³ is —S(═O)₂CH₃. In certain embodiments, R³ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R³ is S(═O)₂aryl (e.g., —S(═O)₂phenyl).

In some embodiments, R³ is —SR^{a3} wherein R^{a3} is as described herein. In certain embodiments, R³ is -Salkyl (e.g., —SCH₃, —SCH₂CH₃, —SPr, —SⁱPr). In certain embodiments, R³ is -Scycloalkyl (e.g., -Scyclopropyl, —Scyclobutyl, —Scyclopentyl, —Scyclohexyl). In certain embodiments, R³ is -Saryl (e.g., -Sphenyl).

In some embodiments, R³ is —S(═O)(═NR^{a3})R^{a3} wherein R^{a3} is as described herein. In certain embodiments, R³ is —S(═O)(═NH)R^{a3} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)^{t}Bu, —S(═O)(═NH)^{t}Bu, —S(═O)(═NH)Cyclopropyl, —S(═O)(═NH)Cyclobutyl). In some embodiments, R³ is —S(═O)(═NCH₃)R^{a3} (e.g., —S(═O)(═NCH₃)CH₃, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R³ is —NR^{a3}S(═O)₂R^{a3} wherein R^{a3} is as described herein. In certain embodiments, R³ is —NHS(═O)₂alkyl (e.g., —NHS(═O)₂CH₃, —NHS(═O)₂CH₂CH₃, —NHS(═O)₂Pr, —NHS(═O)₂ⁱPr). In certain embodiments, R³ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R³ is —N(CH₃) S(═O)₂alkyl (e.g., —N(CH₃) S(═O)₂CH₃, —N(CH₃) S(═O)₂CH₂CH₃, —N(CH₃) S(═O)₂Pr, —N(CH₃) S(═O)₂ⁱPr). In certain embodiments, R³ is —N(CH₃) S(═O)₂cycloalkyl (e.g., —N(CH₃) S(═O)₂cyclopropyl, —N(CH₃) S(═O)₂cyclobutyl, —N(CH₃) S(═O)₂cyclopentyl, —N(CH₃) S(═O)₂cyclohexyl).

In some embodiments, R³ is —S(═O)₂N(R^{a3})₂ wherein R^{a3} is as described herein. (e.g., —S(═O)₂NH₂, —S(═O)₂NHR^{a3}, —S(═O)₂N(CH₃)R^{a3}). In some embodiments, R³ is —S(═O)₂NH₂. In some embodiments, R³ is —S(═O)₂NHR^{a3} (e.g., —S(═O)₂NHCH₃, —S(═O)₂NHCH₂CH₃, —S(═O)₂NHPr, —S(═O)₂NHⁱPr, —S(═O)₂NHcyclopropyl, —S(═O)₂NHcyclobutyl). In some embodiments, R³ is —S(═O)₂N(CH₃)R^{a3} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R^{a1}, R^{a2}, R^{a3} and R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is as defined in any of the embodiments described herein).

In some embodiments, each R^{a1}, R^{a2}, R^{a3} and R^{a4} is independently unsubstituted. In some embodiments, each R^{a1}, R^{a2}, R^{a3} and R^{a4} is independently substituted with 1 instance of R⁵. In some embodiments, each R^{a1}, R^{a2}, R^{a3} and R^{a4} is independently substituted with 2 instances of R⁵. In some embodiments, each R^{a1}, R^{a2}, R^{a3} and R^{a4} is independently substituted with 3 instances of R⁵.

In some embodiments, each R^{a1}, R^{a2}, R^{a3} and R^{a4} is independently selected from the group consisting of H, —C₁-C₆ alkyl, —C₁-C₆ haloalkyl, —C₁-C₆ heteroalkyl substituted with 0 or 1 instances of ═O, C₃-C₉ cycloalkyl, and 3-10 membered heterocyclyl substituted with 0 or 1 instances of ═O, -Me or a combination thereof.

In some embodiments, each R^{a1}, R^{a2}, R^{a3} and R^{a4} is independently selected from the group consisting of H, —C₁-C₆ alkyl, (e.g., -Me, -Et, -Pr, -ⁱPr, -sec-Bu, -^{t}Bu), —C₁-C₆ haloalkyl (e.g., —CF₃, —CHF₂, —CH₂CF₃) and —C₁-C₆ heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃)C(═O)N(CH₃)₂).

In some embodiments, each R^{a1}, R^{a2}, R^{a3} and R^{a4} is independently selected from the group consisting of H, -Me, -Et, -Pr, -ⁱPr, -sec-Bu, -^{t}Bu, —CF₃, —CHF₂, —CH₂CF₃ and —CH₂CH₂N(CH₃)₂.

In some embodiments, each R^{a1}, R^{a2}, R^{a3} and R^{a4} is independently selected from the group consisting of H, —C₁-C₆ alkyl (e.g., -Me, -Et, -Pr, -ⁱPr, -ⁿBu, -^{t}Bu, -sec-Bu, -iso-Bu) and —C₁-C₆ haloalkyl (e.g., —CHF₂, —CF₃). In some embodiments, each R^{a1}, R^{a2}, R^{a3} and R^{a4} is independently selected from the group consisting of H and —C₁-C₆ alkyl (e.g., -Me, -Et, -Pr, -Pr, -ⁿBu, -^{t}Bu, -sec-Bu, -iso-Bu).

In some embodiments, each R^{a1}, R^{a2}, R^{a3} and R^{a4} is independently H.

In some embodiments, each R^{a1}, R^{a2}, R^{a3} and R^{a4} is independently-C₁-C₆ alkyl (e.g., -Me, -Et, -Pr, -ⁱPr, -ⁿBu, -^{t}Bu, -sec-Bu, -iso-Bu). In some embodiments, each R^{a1}, R^{a2}, R^{a3} and R^{a4} is independently-Me. In some embodiments, each R^{a1}, R^{a2}, R^{a3} and R^{a4} is independently-Et. In some embodiments, each R^{a1}, R^{a2}, R^{a3} and R^{a4} is independently-Pr or -ⁱPr.

In some embodiments, each R^{a1}, R^{a2}, R^{a3} and R^{a4} is independently-C₁-C₆ heteroalkyl. In some embodiments, each R^{a1}, R^{a2}, R^{a3} and R^{a4} is independently methoxymethyl (—CH₂OCH₃). In some embodiments, each R^{a1}, R^{a2}, R^{a3} and R^{a4} is independently hydroxymethyl (—CH₂OH). In some embodiments, each R^{a1}, R^{a2}, R^{a3} and R^{a4} is independently aminomethyl (e.g., —CH₂NH₂, —CH₂NHCH₃, —CH₂N(CH₃)₂, —CH₂CH₂N(CH₃)₂). In some embodiments, each R^{a1}, R^{a2}, R^{a3} and R^{a4} is independently-CH₂CH₂N(CH₃)₂).

In some embodiments, each R^{a1}, R^{a2}, R^{a3} and R^{a4} is independently-C₁-C₆ haloalkyl. In some embodiments, each R^{a1}, R^{a2}, R^{a3} and R^{a4} is independently trifluoromethyl (—CF₃). In other embodiments, each R^{a1}, R^{a2}, R^{a3} and R^{a4} is independently difluoromethyl (—CHF₂).

In some embodiments, each R^{a1}, R^{a2}, R^{a3} and R^{a4} is independently C₃-C₉ cycloalkyl (e.g., cyclopropyl, cyclobutyl, cyclopentyl, cyclohexyl). In some embodiments, each R^{a1}, R^{a2}, R^{a3} and R^{a4} is independently cyclopropyl. In some embodiments each R^{a1}, R^{a2}, R^{a3} and R^{a4} is independently cyclobutyl. In some embodiments, each R^{a1}, R^{a2}, R^{a3} and R^{a4} is independently cyclopentyl. In some embodiments, each R^{a1}, R^{a2}, R^{a3} and R^{a4} is independently cyclohexyl. In some embodiments, the cycloalkyl is substituted with 0, 1, 2 or 3 instances of R⁵, wherein each R⁵ is as defined in any of the embodiments described herein.

In some embodiments, each R^{a1}, R^{a2}, R^{a3} and R^{a4} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, each R^{a1}, R^{a2}, R^{a3} and R^{a4} is independently is oxetanyl. In some embodiments, each R^{a1}, R^{a2}, R^{a3} and R^{a4} is independently tetrahydropyranyl. In some embodiments, each R^{a1}, R^{a2}, R^{a3} and R^{a4} is independently tetrahydrofuranyl. In some embodiments, each R^{a1}, R^{a2}, R^{a3} and R^{a4} is independently azetidinyl. In some embodiments, each R^{a1}, R^{a2}, R^{a3} and R^{a4} is independently pyrrolidinyl. In some embodiments, each R^{a1}, R^{a2}, R^{a3} and R^{a4} is independently piperidinyl. In some embodiments, each R^{a1}, R^{a2}, R^{a3} and R^{a4} is independently piperazinyl. In some embodiments, each R^{a1}, R^{a2}, R^{a3} and R^{a4} is independently morpholinyl. In some embodiments, each R^{a1}, R^{a2}, R^{a3} and R^{a4} is independently azepanyl. In some embodiments, each heterocyclyl is substituted with 0, 1, 2 or 3 instances of R⁵, wherein each R⁵ is as defined in any of the embodiments described herein.

In some embodiments, R^{a1}, R^{a2}, R^{a3} and R^{a4} is independently a 5-10 membered heteroaryl (e.g., a 5-6 membered monocyclic heteroaryl or an 8-10 membered bicyclic heteroaryl containing 1-3 heteroatoms independently selected from the group consisting of N, O and S). In some embodiments, R^{a1}, R^{a2}, R^{a3} and R^{a4} is independently a 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms). In some embodiments, R^{a1}, R^{a2}, R^{a3} and R^{a4} is independently a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R^{a1}, R^{a2}, R^{a3} and R^{a4} is independently thiophenyl (e.g., thiophen-2-yl, thiophen-3-yl). In some embodiments, R^{a1}, R^{a2}, R^{a3} and R^{a4} is independently pyrazolyl (e.g., pyrazol-1-yl, pyrazol-3-yl, pyrazol-5-yl). In some embodiments, R^{a1}, R^{a2}, R^{a3} and R^{a4} is independently thiazolyl (e.g., thiazol-2-yl, thiazol-4-yl, thiazol-5-yl). In some embodiments, R^{a1}, R^{a2}, R^{a3} and R^{a4} is independently a 6-membered monocyclic heteroaryl (e.g., pyridyl, pyrimidinyl, triazinyl, pyrazinyl, pyridazinyl). In some embodiments, R^{a1}, R^{a2}, R^{a3} and R^{a4} is independently pyridinyl (e.g., pyridin-2-yl, pyridin-3-yl, pyridin-4-yl). In some embodiments, R^{a1}, R^{a2}, R^{a3} and R^{a4} is independently pyrimidinyl (e.g, pyrimidin-2-yl, pyrimidin-4-yl, pyrimidin-5-yl). In some embodiments, the heteroaryl is substituted with 0, 1, 2 or 3 instances of R⁵, wherein each R⁵ is as defined in any of the embodiments described herein.

In some embodiments, R^{a1}, R^{a2}, R^{a3} and R^{a4} is independently C₆-C₁₀ aryl. In some embodiments, R^{a1}, R^{a2}, R^{a3} and R^{a4} is independently 6-10 membered mono or bicyclic aryl. In some embodiments, R^{a1}, R^{a2}, R^{a3} and R^{a4} is independently phenyl. In some embodiments, the phenyl is substituted with 0, 1, 2 or 3 instances of R⁵ as defined in any of the embodiments described herein. In some embodiments, the aryl is substituted with 0, 1, 2 or 3 instances of R⁵, wherein each R⁵ is as defined in any of the embodiments described herein.

In some embodiments, each R^{a1}, R^{a2}, R^{a3} and R^{a4} is independently cycloalkylalkyl (e.g., cyclopropylmethyl, cyclobutylmethyl, cyclopentylmethyl, cyclohexylmethyl, cycloheptylmethyl). In some embodiments, the cycloalkylalkyl is substituted with 0, 1, 2 or 3 instances of R⁵, wherein each R⁵ is as defined in any of the embodiments described herein.

In some embodiments, each R^{a1}, R^{a2}, R^{a3} and R^{a4} is independently heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl). In some embodiments, the heterocyclylalkyl is substituted with 0, 1, 2 or 3 instances of R⁵, wherein each R⁵ is as defined in any of the embodiments described herein.

In some embodiments, each R^{a1}, R^{a2}, R^{a3} and R^{a4} is independently arylalkyl. In some embodiments, each R^{a1}, R^{a2}, R^{a3} and R^{a4} is independently benzyl. In some embodiments, the arylalkyl is substituted with 0, 1, 2 or 3 instances of R⁵, wherein each R⁵ is as defined in any of the embodiments described herein.

In some embodiments, each R^{a1}, R^{a2}, R^{a3} and R^{a4} is independently heteroarylalkyl (e.g., pyridinylmethyl, thiazolylmethyl, triazolylmethyl, pyrazolylmethyl). In some embodiments, the heteroarylalkyl is substituted with 0, 1, 2 or 3 instances of R⁵, wherein each R⁵ is as defined in any of the embodiments described herein.

As generally defined herein, each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu). and C₃-C₉ cycloalkyl (e.g., cyclopropyl, cyclobutyl, cyclopentyl, cyclohexyl).

In some embodiments, each R⁵ is independently selected from the group consisting of ═O, halo, —CN, —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂), —C₃-C₉ cycloalkyl (e.g., cyclopropyl, cyclobutyl, cyclopentyl, cyclohexyl), 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl), C₆-C₁₀ aryl (e.g., phenyl), 5-10 membered heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl), cycloalkylalkyl (e.g., cyclopropylmethyl, cyclobutylmethyl, cyclopentylmethyl, cyclohexylmethyl, cycloheptylmethyl), heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl), arylalkyl (e.g., benzyl), heteroarylalkyl (e.g., pyridinylmethyl, thiazolylmethyl, triazolylmethyl, pyrazolylmethyl), —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is as defined in any of the embodiments described herein. In some embodiments, each R^{b} is H or -Me. In some embodiments, R⁵ is -Me.

As generally defined herein, each R⁶ is independently selected from the group consisting of —C₁-C₆ alkyl, —C₃-C₉ cycloalkyl, 3-10 membered heterocyclyl, —OH, ═O, halo, —OC₁-C₆ alkyl, —C₁-C₆ haloalkyl, —C(═O)C₁-C₆ alkyl, —N(C₁-C₆ alkyl)₂, —C₁-C₆ heteroalkyl, and —NHC(═O)C₁-C₆ alkyl, wherein one or more hydrogens of the —C₁-C₆ alkyl can be replaced with deuterium.

In some embodiments, each R⁶ is independently selected from the group consisting of -Me, -Et, -Pr, -CD₃, cyclopropyl, oxetan-3-yl, —OH, ═O, —F, —OCH₃, —CH₂CH₂F, —CH₂CHF₂, —CH₂CH₂CF₃, —C(═O)CH₃, —N(CH₃)₂, —CH₂N(CH₃)₂, —CH₂N(CH₃)CH₂CH₃, —N(Pr)(CH₂CH₃), —N(Pr)(CH₃), —N(CH₂CH₃)₂, —N(CH₃)(CH₂CH₃), —NHC(═O)CH₃).

In some embodiments, each R⁶ is independently selected from the group consisting of -Me, -Et, -CD₃, cyclopropyl, oxetan-3-yl, —OH, ═O, —N(CH₃)₂ and —CH₂N(CH₃)₂.

In some embodiments, R⁶ is ═O.

In certain embodiments, R⁶ is halo (e.g., fluoro, chloro, bromo, iodo). In some embodiments, R⁶ is —Cl. In some embodiments, R⁶ is —F. In some embodiments, R⁶ is —Br. In some embodiments, R⁶ is —I.

In certain embodiments, R⁶ is —C₁-C₆ alkyl. In some embodiments, one or more hydrogens of the —C₁-C₆ alkyl can be replaced with deuterium. In some embodiments, R⁶ is -Me. In some embodiments, R⁶ is -CD₃. In some embodiments, R⁶ is -Et. In some embodiments R⁶ is -Pr or -iPr.

In some embodiments, R⁶ is C₃-C₉ cycloalkyl (e.g., cyclopropyl, cyclobutyl, cyclopentyl, cyclohexyl). In some embodiments, R⁶ is cyclopropyl. In some embodiments R⁶ is cyclobutyl. In some embodiments, R⁶ is cyclopentyl. In some embodiments, R⁶ is cyclohexyl.

In some embodiments, R⁶ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁶ is 3-6 membered heterocyclyl (e.g., oxetanyl, tetrahydropyranyl, tetrahydrofuranyl, azetidinyl, pyrrolidinyl, piperidinyl, piperazinyl, morpholinyl). In some embodiments, R⁶ is oxetanyl (e.g., oxetan-3-yl). In some embodiments, R⁶ is tetrahydropyranyl. In some embodiments, R⁶ is tetrahydrofuranyl. In some embodiments, R⁶ is azetidinyl. In some embodiments, R⁶ is pyrrolidinyl. In some embodiments, R⁶ is piperidinyl. In some embodiments, R⁶ is piperazinyl. In some embodiments, R⁶ is morpholinyl. In some embodiments, R⁶ is azepanyl.

In some embodiments, R⁶ is OH.

In some embodiments, R⁶ is —OC₁-C₆ alkyl (e.g., methoxy, ethoxy, propoxy, isopropoxy). In some embodiments, R⁶ is methoxy. In some embodiments, R⁶ is ethoxy. In some embodiments, R⁶ is propoxy. In some embodiments, R⁶ is isopropoxy.

In some embodiments, R⁶ is —C₁-C₆ haloalkyl (e.g., —CF₃, —CHF₂, —CH₂F, —CH₂CF₃). In some embodiments, R⁶ is trifluoromethyl (—CF₃). In other embodiments, R⁶ is difluoromethyl (—CHF₂).

In some embodiments, R⁶ is —C(═O)C₁-C₆ alkyl (e.g., —C(═O)CH₃, —C(═O)ⁱPr, —C(═O)CH₂CH₂CH₃, —C(O)ⁱBu)).

In some embodiments, R⁶ is —N(C₁-C₆ alkyl)₂ (e.g., —N(CH₃)₂, —N(CH₃)CH₂CH₃, —N(CH₃)CH₂CH₂CH₃, —N(CH₃)ⁱPr). In some embodiments, R⁶ is —N(CH₃)₂.

In some embodiments, R⁶ is —C₁-C₆ heteroalkyl. In some embodiments, R⁶ is methoxymethyl (—CH₂OCH₃). In some embodiments, R⁶ is hydroxymethyl (—CH₂OH). In some embodiments, R⁶ is aminomethyl (e.g., —CH₂NH₂, —CH₂NHCH₃, —CH₂N(CH₃)₂.

In some embodiments, R⁶ is —NHC(═O)C₁-C₆ alkyl (e.g., —NHC(═O)CH₃, —NHC(═O)CH₂CH₃, —NHC(═O)CH₂CH₂CH₃, —NHC(═O)ⁱPr, —NHC(═O)Bu, —NHC(═O)^{t}Bu).

In one embodiment, provided is a compound selected from the compounds of Table 1, or pharmaceutically acceptable salts thereof.

Compounds described herein (e.g., a compound of Formula (A) and Formula (I) to (III7a) or a compound of Table 1, or pharmaceutically acceptable salts thereof) are useful as inhibitors of PRMT5 (e.g., MTA uncompetitive PRMT5 inhibitors).

Table 1 indicates IC₅₀ and IC₉₀ values in an MTAP-isogenic cell line pair for exemplary compounds in an SDMA in-cell western assay (described in Example 246) (columns 4-6). HAP1 MTAP-intact is a cell line in which endogenous levels of MTAP are expressed, and HAP1 MTAP-deleted is an MTAP-null cell line. For Table 1, “a” and “aa” indicates an IC₅₀ of <5 nM, “b” and “bb” indicates an IC₅₀ equal to or greater than 5 nM but less than 50 nM, and “c” and “cc” indicates an IC₅₀ of greater than or equal to 50 nM in the HAP1 MTAP-intact (column 4) and the HAP1 MTAP-deleted (column 5) assays, respectively. Similarly, “aaa” indicates an IC₉₀ of <75 nM, “bbb” indicates an IC₉₀ equal to or greater than 75 nM but less than 125 nM, and “ccc” indicates an IC₉₀ of greater than or equal to 125 nM in the HAP1 MTAP-deleted (column 6) assay.

In column 7, “A” indicates an IC₅₀ ratio greater than or equal to 30 fold between the IC₅₀ in the HAP1 MTAP-intact cell line and the HAP1 MTAP-deleted cell line; “B” indicates an IC₅₀ ratio greater than or equal to 15 fold but lower than 30 fold between the IC₅₀ in the HAP1 MTAP-intact cell line and the HAP1 MTAP-deleted cell line; “C” indicates an IC₅₀ ratio of less than 15 fold between the IC₅₀ in the HAP1 MTAP-intact cell line and the HAP1 MTAP-deleted cell line. Compounds with a ratio in the SDMA in-cell western assay of equal to or greater than 3 fold are considered MTAP-selective.

Table 1 additionally indicates IC₅₀ values in a viability assay for the MTAP-deleted cell line (described in Example 247)(column 8), indicating the effect of treatment with compound on cell survival. In column 8, a value of A* indicates an IC₅₀ of less than 100 nM, a value of B* indicates an IC₅₀ equal to or greater than 100 nM but less than 1 μM, and a value of C* indicates an IC₅₀ greater than or equal to 1 μM.

Unless otherwise indicated, the absolute stereochemistry of all chiral atoms is as depicted. Compounds marked with (or) or (rel) are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds marked with (abs) are single enantiomers wherein the absolute stereochemistry is as indicated. In some instances, different indicators selected from (abs) (or) and (and) apply to different portions of the molecule. For example, compound 2 is described as “(abs) core piperidine, (or) R³ pyrrolidine”. The “(abs) core piperidine” designation indicates that the absolute stereochemistry of the piperidine attached to the oxamide of Formula (I) is as depicted. The “(or)R³ pyrrolidine” designation indicates that the stereochemistry on the pyrrolidine corresponding to R³ of Formula (I) is either R or S (i.e., not a mixture of R and S), but the absolute configuration has been arbitrarily assigned.

TABLE 1
Exemplary compounds and biological data

| | | | | | | Hap1 | Hap1 |
|---|---|---|---|---|---|---|---|
| | | | Hap1 | Hap1 | Hap1 | MTAP | MTAP |
| | | | MTAP | MTAP | MTAP | intact/ | deleted |
| | | | intact | deleted | deleted | deleted | viability |
| STRUCTURE | Nr | Stereochemistry | IC50 | IC50 | IC90 | ratio | IC50 |
| 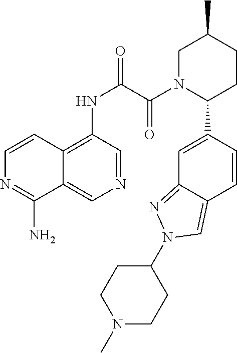 [SMILES: C[C@H]1CC[C@H](c2ccc3cn(C4CCN(C)CC4)nc3c2)N(C(=O)C(=O)Nc2cncc3c(N)nccc23)C1] | 1 | (abs) | c | aa | aaa | A | A* |
| 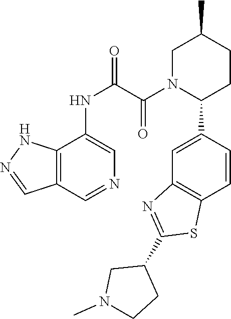 [SMILES: C[C@H]1CC[C@H](c2ccc3sc([C@@H]4CCN(C)C4)nc3c2)N(C(=O)C(=O)Nc2cncc3cn[nH]c23)C1] | 2 | (abs) piperidine, (or) R³ pyrrolidine | b | aa | aaa | C | A* |
| 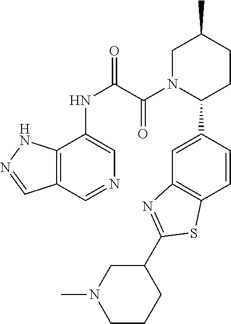 [SMILES: C[C@H]1CC[C@H](c2ccc3sc(C4CCCN(C)C4)nc3c2)N(C(=O)C(=O)Nc2cncc3cn[nH]c23)C1] | 3 | (abs) core piperidine | b | aa | aaa | C | A* |
| 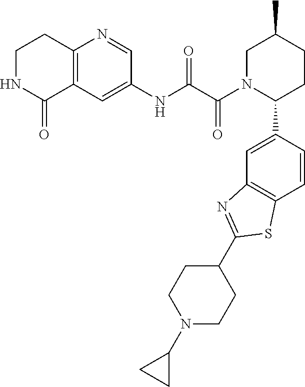 [SMILES: C[C@H]1CC[C@H](c2ccc3sc(C4CCN(C5CC5)CC4)nc3c2)N(C(=O)C(=O)Nc2cnc3c(c2)C(=O)NCC3)C1] | 4 | (abs) | c | bb | bbb | C | B* |
| 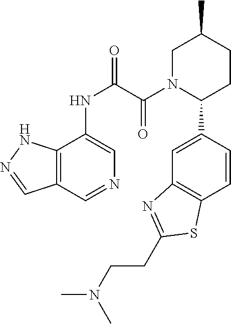 [SMILES: C[C@H]1CC[C@H](c2ccc3sc(CCN(C)C)nc3c2)N(C(=O)C(=O)Nc2cncc3cn[nH]c23)C1] | 5 | (abs) | b | aa | aaa | B | A* |
| 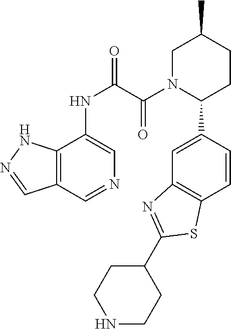 [SMILES: C[C@H]1CC[C@H](c2ccc3sc(C4CCNCC4)nc3c2)N(C(=O)C(=O)Nc2cncc3cn[nH]c23)C1] | 6 | (abs) | b | bb | aaa | C | A* |
| 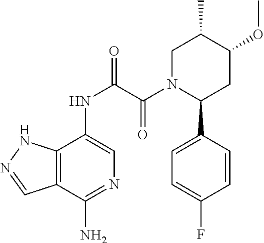 [SMILES: CO[C@@H]1C[C@@H](c2ccc(F)cc2)N(C(=O)C(=O)Nc2cnc(N)c3cn[nH]c23)C[C@@H]1C] | 7 | (or) | b | aa | aaa | B | A* |
| 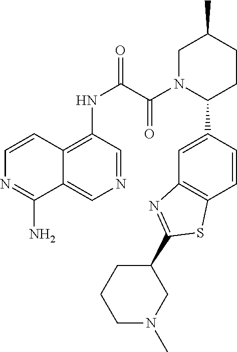 [SMILES: C[C@H]1CC[C@H](c2ccc3sc([C@@H]4CCCN(C)C4)nc3c2)N(C(=O)C(=O)Nc2cncc3c(N)nccc23)C1] | 8 | (and) core piperidine, or R³ piperidine | c | aa | aaa | A | A* |
| 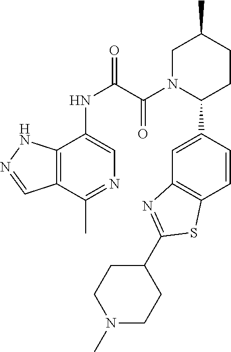 [SMILES: Cc1ncc(NC(=O)C(=O)N2C[C@@H](C)CC[C@@H]2c2ccc3sc(C4CCN(C)CC4)nc3c2)c2[nH]ncc12] | 9 | (abs) | b | aa | aaa | C | A* |
| 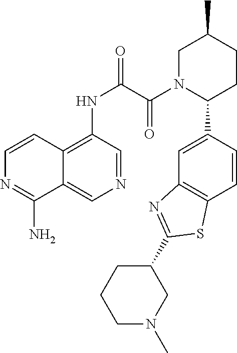 [SMILES: C[C@H]1CC[C@H](c2ccc3sc([C@H]4CCCN(C)C4)nc3c2)N(C(=O)C(=O)Nc2cncc3c(N)nccc23)C1] | 10 | (and) core piperidine, (or) R³ piperidine | c | aa | aaa | A | A* |
| 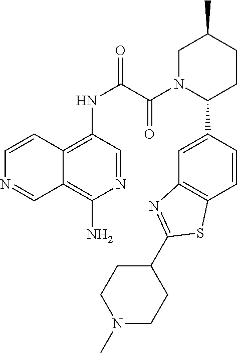 [SMILES: C[C@H]1CC[C@H](c2ccc3sc(C4CCN(C)CC4)nc3c2)N(C(=O)C(=O)Nc2cnc(N)c3cnccc23)C1] | 11 | (abs) | b | aa | aaa | C | A* |
| 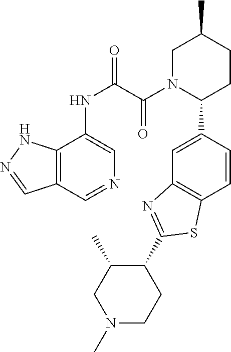 [SMILES: C[C@H]1CC[C@H](c2ccc3sc([C@@H]4CCN(C)C[C@@H]4C)nc3c2)N(C(=O)C(=O)Nc2cncc3cn[nH]c23)C1] | 12 | (abs) core piperidine, (and) R³ piperidine | c | aa | aaa | B | A* |
| 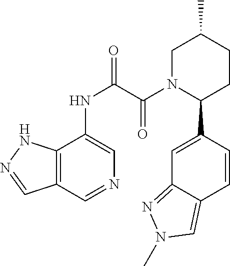 [SMILES: C[C@@H]1CC[C@@H](c2ccc3cn(C)nc3c2)N(C(=O)C(=O)Nc2cncc3cn[nH]c23)C1] | 13 | (or) | c | bb | bbb | B | B* |
| 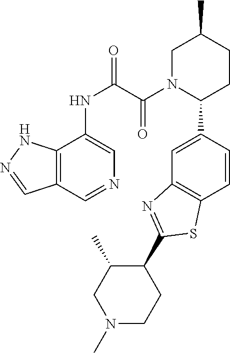 [SMILES: C[C@H]1CC[C@H](c2ccc3sc([C@H]4CCN(C)C[C@@H]4C)nc3c2)N(C(=O)C(=O)Nc2cncc3cn[nH]c23)C1] | 14 | (abs) core piperidine, (or) R³ piperidine | b | aa | aaa | B | A* |
| 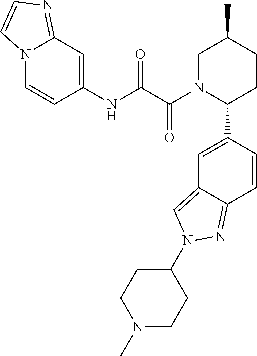 [SMILES: C[C@H]1CC[C@H](c2ccc3nn(C4CCN(C)CC4)cc3c2)N(C(=O)C(=O)Nc2ccn3ccnc3c2)C1] | 15 | (abs) | c | aa | bbb | A | A* |
| 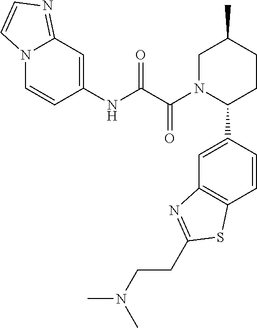 [SMILES: C[C@H]1CC[C@H](c2ccc3sc(CCN(C)C)nc3c2)N(C(=O)C(=O)Nc2ccn3ccnc3c2)C1] | 16 | (abs) | c | aa | aaa | A | A* |
| 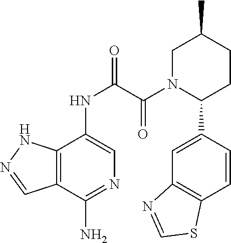 [SMILES: C[C@H]1CC[C@H](c2ccc3scnc3c2)N(C(=O)C(=O)Nc2cnc(N)c3cn[nH]c23)C1] | 17 | (abs) | b | aa | aaa | B | A* |
| 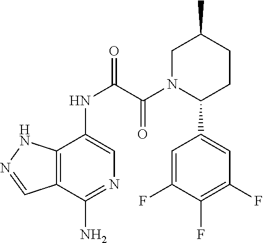 [SMILES: C[C@H]1CC[C@H](c2cc(F)c(F)c(F)c2)N(C(=O)C(=O)Nc2cnc(N)c3cn[nH]c23)C1] | 18 | (abs) | c | aa | aaa | A | A* |
| 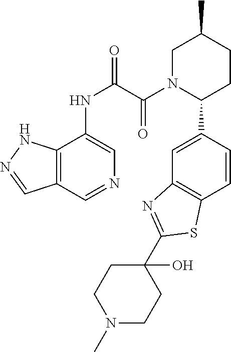 [SMILES: C[C@H]1CC[C@H](c2ccc3sc(C4(O)CCN(C)CC4)nc3c2)N(C(=O)C(=O)Nc2cncc3cn[nH]c23)C1] | 19 | (abs) | b | aa | aaa | C | A* |
| 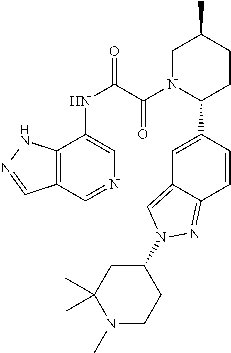 [SMILES: C[C@H]1CC[C@H](c2ccc3nn([C@@H]4CCN(C)C(C)(C)C4)cc3c2)N(C(=O)C(=O)Nc2cncc3cn[nH]c23)C1] | 20 | (abs) core piperidine, (or) R³ piperidine | b | aa | aaa | C | A* |
| 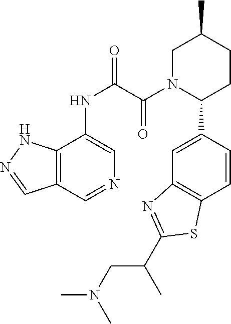 [SMILES: CC(CN(C)C)c1nc2cc([C@H]3CC[C@H](C)CN3C(=O)C(=O)Nc3cncc4cn[nH]c34)ccc2s1] | 21 | (abs) piperidine | b | aa | aaa | C | A* |
| 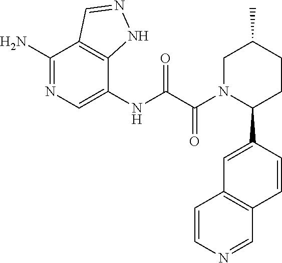 [SMILES: C[C@@H]1CC[C@@H](c2ccc3cnccc3c2)N(C(=O)C(=O)Nc2cnc(N)c3cn[nH]c23)C1] | 22 | (and) | b | aa | aaa | C | A* |
| 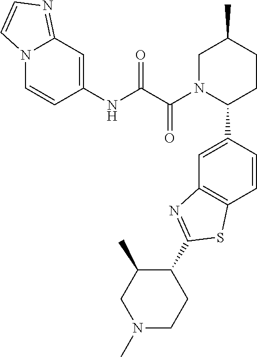 [SMILES: C[C@H]1CC[C@H](c2ccc3sc([C@@H]4CCN(C)C[C@H]4C)nc3c2)N(C(=O)C(=O)Nc2ccn3ccnc3c2)C1] | 23 | (abs) core piperidine, (or) R³ piperidine | c | aa | aaa | A | A* |
| 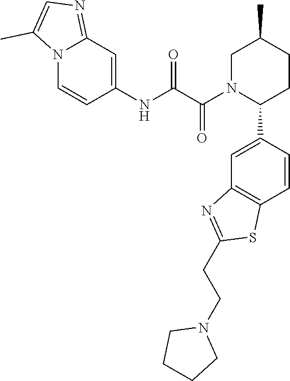 [SMILES: Cc1cnc2cc(NC(=O)C(=O)N3C[C@@H](C)CC[C@@H]3c3ccc4sc(CCN5CCCC5)nc4c3)ccn12] | 24 | (abs) | c | bb | bbb | A | B* |
| 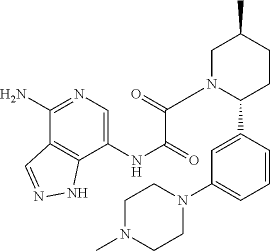 [SMILES: C[C@H]1CC[C@H](c2cccc(N3CCN(C)CC3)c2)N(C(=O)C(=O)Nc2cnc(N)c3cn[nH]c23)C1] | 25 | (abs) | b | aa | aaa | C | A* |
| 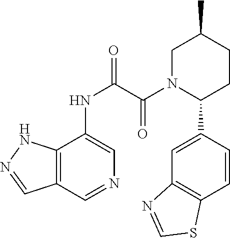 [SMILES: C[C@H]1CC[C@H](c2ccc3scnc3c2)N(C(=O)C(=O)Nc2cncc3cn[nH]c23)C1] | 26 | (abs) | c | bb | ccc | A | B* |
| 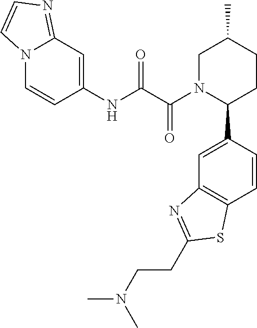 [SMILES: C[C@@H]1CC[C@@H](c2ccc3sc(CCN(C)C)nc3c2)N(C(=O)C(=O)Nc2ccn3ccnc3c2)C1] | 27 | (abs) | c | cc | ccc | C | C* |
| 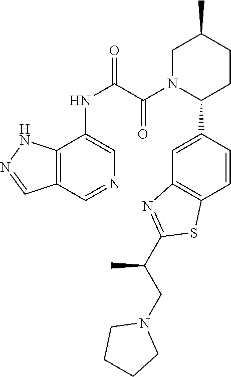 [SMILES: C[C@H]1CC[C@H](c2ccc3sc([C@H](C)CN4CCCC4)nc3c2)N(C(=O)C(=O)Nc2cncc3cn[nH]c23)C1] | 28 | (abs) piperidine, (or) R³ side chain | b | aa | aaa | C | A* |
| 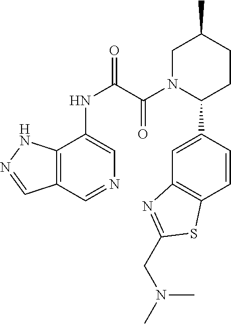 [SMILES: C[C@H]1CC[C@H](c2ccc3sc(CN(C)C)nc3c2)N(C(=O)C(=O)Nc2cncc3cn[nH]c23)C1] | 29 | (abs) | c | bb | bbb | B | B* |
| 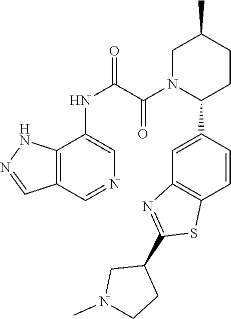 [SMILES: C[C@H]1CC[C@H](c2ccc3sc([C@H]4CCN(C)C4)nc3c2)N(C(=O)C(=O)Nc2cncc3cn[nH]c23)C1] | 30 | (abs) piperidine, (or) R³ pyrrolidine | b | aa | aaa | B | A* |
| 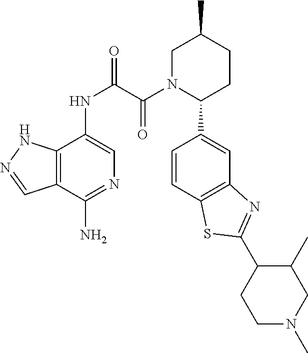 [SMILES: CC1CN(C)CCC1c1nc2cc([C@H]3CC[C@H](C)CN3C(=O)C(=O)Nc3cnc(N)c4cn[nH]c34)ccc2s1] | 31 | (abs) core piperidine | a | aa | aaa | C | A* |
| 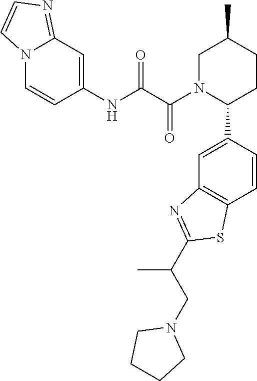 [SMILES: CC(CN1CCCC1)c1nc2cc([C@H]3CC[C@H](C)CN3C(=O)C(=O)Nc3ccn4ccnc4c3)ccc2s1] | 32 | (abs) piperidine | c | aa | bbb | A | A* |
| 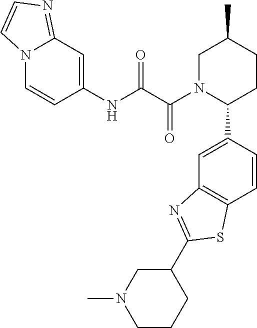 [SMILES: C[C@H]1CC[C@H](c2ccc3sc(C4CCCN(C)C4)nc3c2)N(C(=O)C(=O)Nc2ccn3ccnc3c2)C1] | 33 | (abs) core piperidine | b | aa | aaa | C | A* |
| 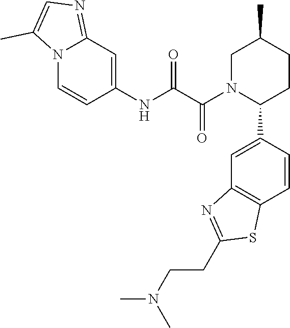 [SMILES: Cc1cnc2cc(NC(=O)C(=O)N3C[C@@H](C)CC[C@@H]3c3ccc4sc(CCN(C)C)nc4c3)ccn12] | 34 | (abs) | c | bb | bbb | A | A* |
| 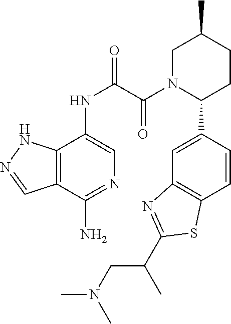 [SMILES: CC(CN(C)C)c1nc2cc([C@H]3CC[C@H](C)CN3C(=O)C(=O)Nc3cnc(N)c4cn[nH]c34)ccc2s1] | 35 | (abs) piperidine | b | aa | aaa | C | A* |
| 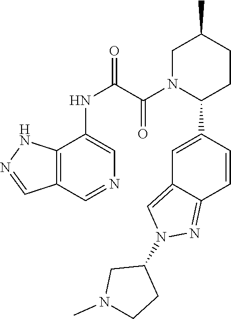 [SMILES: C[C@H]1CC[C@H](c2ccc3nn([C@@H]4CCN(C)C4)cc3c2)N(C(=O)C(=O)Nc2cncc3cn[nH]c23)C1] | 36 | (abs) | c | aa | bbb | A | B* |
| 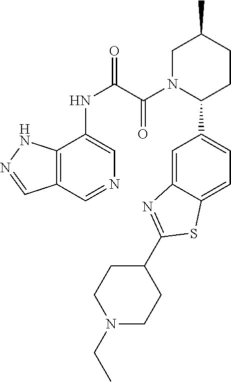 [SMILES: CCN1CCC(c2nc3cc([C@H]4CC[C@H](C)CN4C(=O)C(=O)Nc4cncc5cn[nH]c45)ccc3s2)CC1] | 37 | (abs) | b | aa | aaa | B | A* |
| 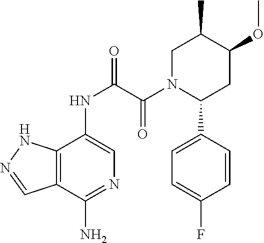 [SMILES: CO[C@H]1C[C@H](c2ccc(F)cc2)N(C(=O)C(=O)Nc2cnc(N)c3cn[nH]c23)C[C@H]1C] | 38 | (or) | c | cc | ccc | C | B* |
| 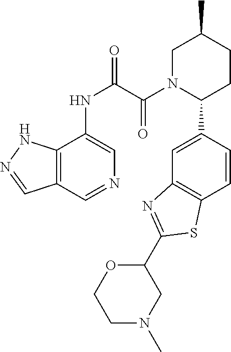 [SMILES: C[C@H]1CC[C@H](c2ccc3sc(C4CN(C)CCO4)nc3c2)N(C(=O)C(=O)Nc2cncc3cn[nH]c23)C1] | 39 | (abs) piperidine | c | bb | ccc | B | A* |
| 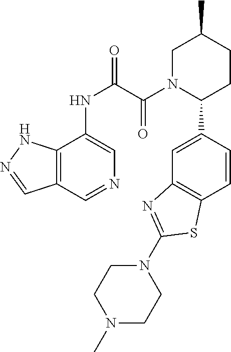 [SMILES: C[C@H]1CC[C@H](c2ccc3sc(N4CCN(C)CC4)nc3c2)N(C(=O)C(=O)Nc2cncc3cn[nH]c23)C1] | 40 | (abs) | b | aa | aaa | C | A* |
| 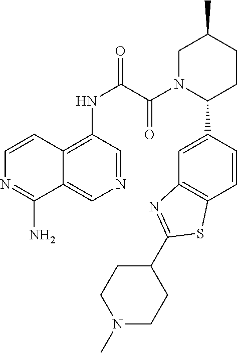 [SMILES: C[C@H]1CC[C@H](c2ccc3sc(C4CCN(C)CC4)nc3c2)N(C(=O)C(=O)Nc2cncc3c(N)nccc23)C1] | 41 | (abs) | c | aa | aaa | A | A* |
| 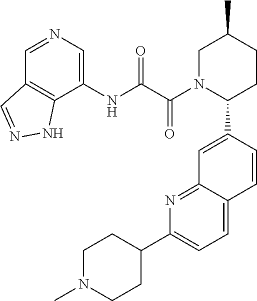 [SMILES: C[C@H]1CC[C@H](c2ccc3ccc(C4CCN(C)CC4)nc3c2)N(C(=O)C(=O)Nc2cncc3cn[nH]c23)C1] | 42 | (abs) | c | aa | aaa | A | A* |
| 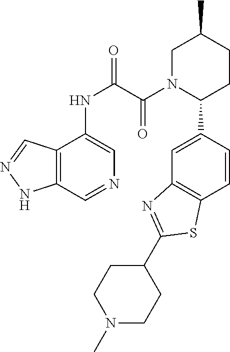 [SMILES: C[C@H]1CC[C@H](c2ccc3sc(C4CCN(C)CC4)nc3c2)N(C(=O)C(=O)Nc2cncc3[nH]ncc23)C1] | 43 | (abs) | b | aa | aaa | C | A* |
| 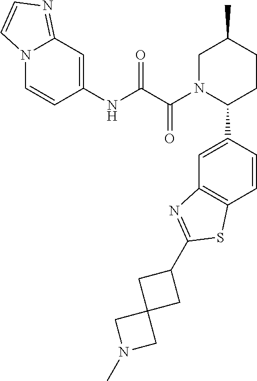 [SMILES: C[C@H]1CC[C@H](c2ccc3sc(C4CC5(C4)CN(C)C5)nc3c2)N(C(=O)C(=O)Nc2ccn3ccnc3c2)C1] | 44 | (abs) | c | aa | aaa | B | A* |
| 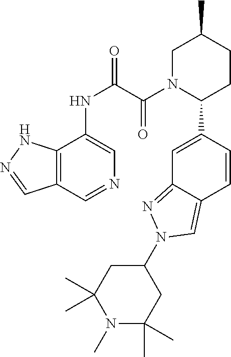 [SMILES: C[C@H]1CC[C@H](c2ccc3cn(C4CC(C)(C)N(C)C(C)(C)C4)nc3c2)N(C(=O)C(=O)Nc2cncc3cn[nH]c23)C1] | 45 | (abs) | b | aa | aaa | B | A* |
| 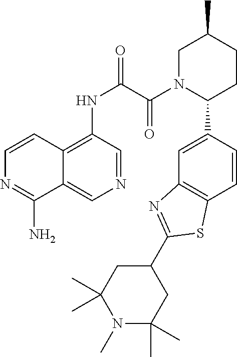 [SMILES: C[C@H]1CC[C@H](c2ccc3sc(C4CC(C)(C)N(C)C(C)(C)C4)nc3c2)N(C(=O)C(=O)Nc2cncc3c(N)nccc23)C1] | 46 | (abs) | c | aa | aaa | A | A* |
| 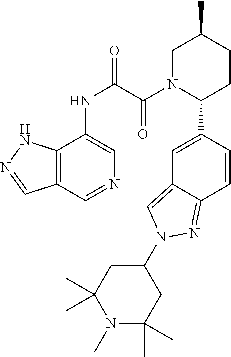 [SMILES: C[C@H]1CC[C@H](c2ccc3nn(C4CC(C)(C)N(C)C(C)(C)C4)cc3c2)N(C(=O)C(=O)Nc2cncc3cn[nH]c23)C1] | 47 | (abs) | b | aa | aaa | B | A* |
| 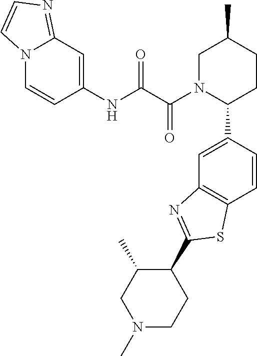 [SMILES: C[C@H]1CC[C@H](c2ccc3sc([C@H]4CCN(C)C[C@@H]4C)nc3c2)N(C(=O)C(=O)Nc2ccn3ccnc3c2)C1] | 48 | (abs) core piperidine, (and) R³ piperidine | c | aa | aaa | A | A* |
| 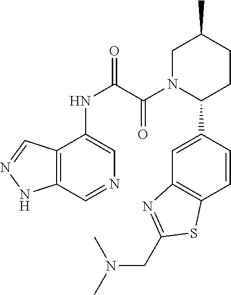 [SMILES: C[C@H]1CC[C@H](c2ccc3sc(CN(C)C)nc3c2)N(C(=O)C(=O)Nc2cncc3[nH]ncc23)C1] | 49 | (abs) | c | bb | bbb | B | B* |
| 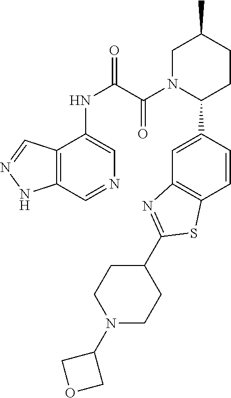 [SMILES: C[C@H]1CC[C@H](c2ccc3sc(C4CCN(C5COC5)CC4)nc3c2)N(C(=O)C(=O)Nc2cncc3[nH]ncc23)C1] | 50 | (abs) | c | bb | ccc | B | B* |
| 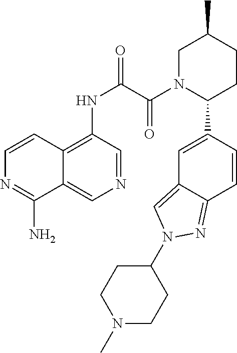 [SMILES: C[C@H]1CC[C@H](c2ccc3nn(C4CCN(C)CC4)cc3c2)N(C(=O)C(=O)Nc2cncc3c(N)nccc23)C1] | 51 | (abs) | c | bb | ccc | A | B* |
| 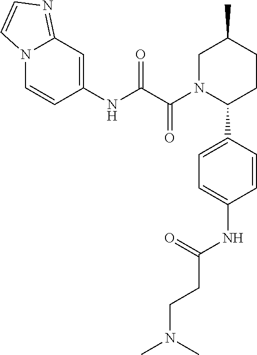 [SMILES: C[C@H]1CC[C@H](c2ccc(NC(=O)CCN(C)C)cc2)N(C(=O)C(=O)Nc2ccn3ccnc3c2)C1] | 52 | (abs) | c | bb | ccc | B | B* |
| 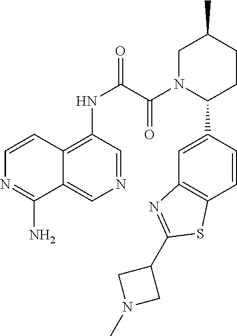 [SMILES: C[C@H]1CC[C@H](c2ccc3sc(C4CN(C)C4)nc3c2)N(C(=O)C(=O)Nc2cncc3c(N)nccc23)C1] | 53 | (abs) | c | bb | ccc | A | A* |
| 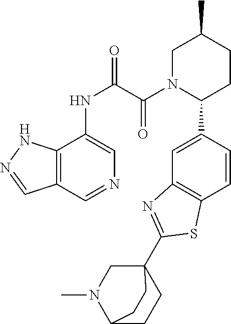 [SMILES: C[C@H]1CC[C@H](c2ccc3sc(C45CCC(CC4)N(C)C5)nc3c2)N(C(=O)C(=O)Nc2cncc3cn[nH]c23)C1] | 54 | (abs) | b | aa | aaa | B | A* |
| 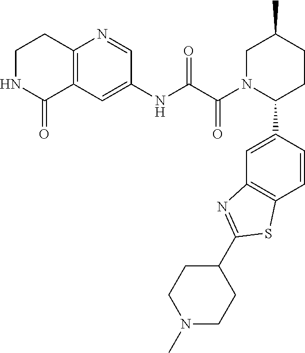 [SMILES: C[C@H]1CC[C@H](c2ccc3sc(C4CCN(C)CC4)nc3c2)N(C(=O)C(=O)Nc2cnc3c(c2)C(=O)NCC3)C1] | 55 | (abs) | b | aa | aaa | C | A* |
| 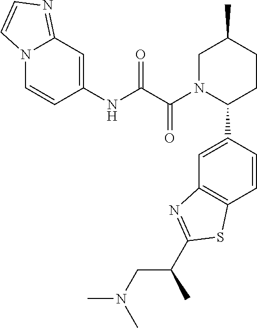 [SMILES: C[C@H]1CC[C@H](c2ccc3sc([C@@H](C)CN(C)C)nc3c2)N(C(=O)C(=O)Nc2ccn3ccnc3c2)C1] | 56 | (abs) core piperidine, (or) R³ | c | aa | bbb | B | A* |
| 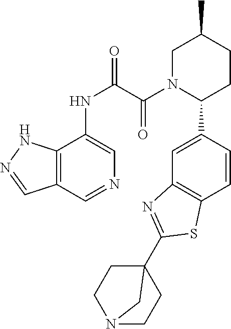 [SMILES: C[C@H]1CC[C@H](c2ccc3sc(C45CCN(CC4)C5)nc3c2)N(C(=O)C(=O)Nc2cncc3cn[nH]c23)C1] | 57 | (abs) | b | aa | aaa | C | A* |
| 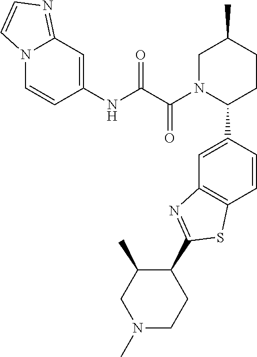 [SMILES: C[C@H]1CC[C@H](c2ccc3sc([C@H]4CCN(C)C[C@H]4C)nc3c2)N(C(=O)C(=O)Nc2ccn3ccnc3c2)C1] | 58 | (abs) core piperidine, (or) R³ piperidine | c | aa | aaa | A | A* |
| 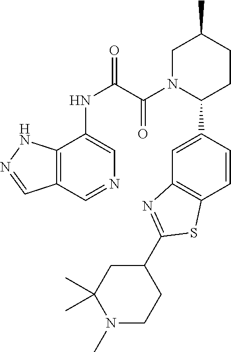 [SMILES: C[C@H]1CC[C@H](c2ccc3sc(C4CCN(C)C(C)(C)C4)nc3c2)N(C(=O)C(=O)Nc2cncc3cn[nH]c23)C1] | 59 | (abs) core piperidine | b | aa | aaa | C | A* |
| 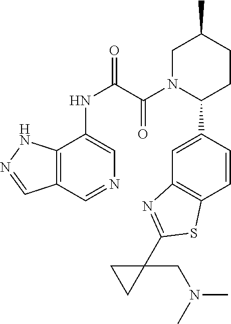 [SMILES: C[C@H]1CC[C@H](c2ccc3sc(C4(CN(C)C)CC4)nc3c2)N(C(=O)C(=O)Nc2cncc3cn[nH]c23)C1] | 60 | (abs) | c | aa | aaa | B | A* |
| 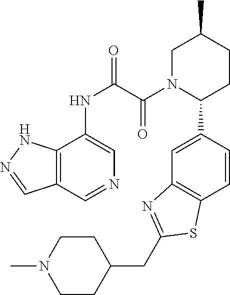 [SMILES: C[C@H]1CC[C@H](c2ccc3sc(CC4CCN(C)CC4)nc3c2)N(C(=O)C(=O)Nc2cncc3cn[nH]c23)C1] | 61 | (abs) | b | aa | aaa | B | A* |
| 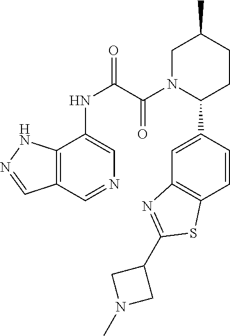 [SMILES: C[C@H]1CC[C@H](c2ccc3sc(C4CN(C)C4)nc3c2)N(C(=O)C(=O)Nc2cncc3cn[nH]c23)C1] | 62 | (abs) | b | aa | aaa | B | A* |
| 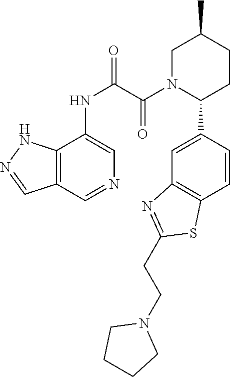 [SMILES: C[C@H]1CC[C@H](c2ccc3sc(CCN4CCCC4)nc3c2)N(C(=O)C(=O)Nc2cncc3cn[nH]c23)C1] | 63 | (abs) | c | aa | aaa | A | A* |
| 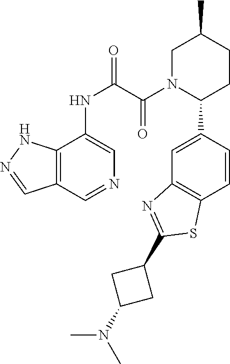 [SMILES: C[C@H]1CC[C@H](c2ccc3sc([C@H]4C[C@H](N(C)C)C4)nc3c2)N(C(=O)C(=O)Nc2cncc3cn[nH]c23)C1] | 64 | (abs) | b | aa | aaa | B | A* |
| 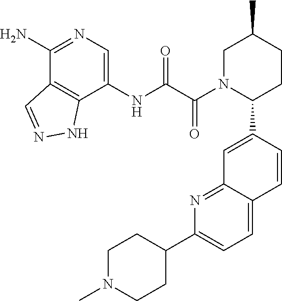 [SMILES: C[C@H]1CC[C@H](c2ccc3ccc(C4CCN(C)CC4)nc3c2)N(C(=O)C(=O)Nc2cnc(N)c3cn[nH]c23)C1] | 65 | (abs) | b | bb | aaa | C | A* |
| 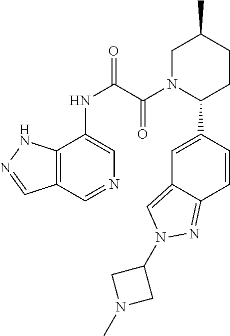 [SMILES: C[C@H]1CC[C@H](c2ccc3nn(C4CN(C)C4)cc3c2)N(C(=O)C(=O)Nc2cncc3cn[nH]c23)C1] | 66 | (abs) | c | bb | ccc | A | B* |
| 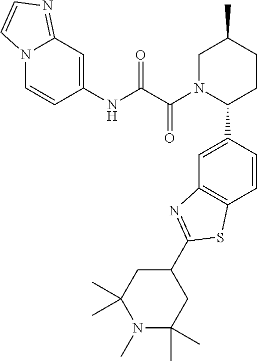 [SMILES: C[C@H]1CC[C@H](c2ccc3sc(C4CC(C)(C)N(C)C(C)(C)C4)nc3c2)N(C(=O)C(=O)Nc2ccn3ccnc3c2)C1] | 67 | (abs) | b | aa | aaa | B | A* |
| 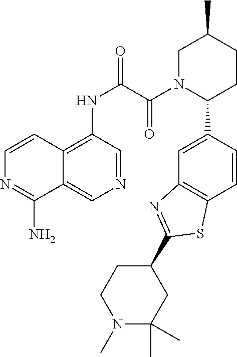 [SMILES: C[C@H]1CC[C@H](c2ccc3sc([C@@H]4CCN(C)C(C)(C)C4)nc3c2)N(C(=O)C(=O)Nc2cncc3c(N)nccc23)C1] | 68 | (abs) core piperidine, (or) R³ piperidine | c | aa | aaa | A | A* |
| 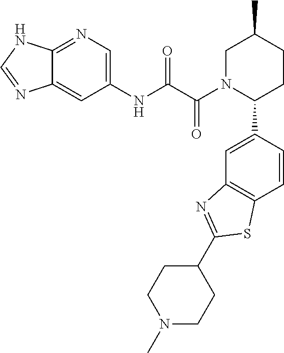 [SMILES: C[C@H]1CC[C@H](c2ccc3sc(C4CCN(C)CC4)nc3c2)N(C(=O)C(=O)Nc2cnc3[nH]cnc3c2)C1] | 69 | (abs) | c | bb | bbb | A | A* |
| 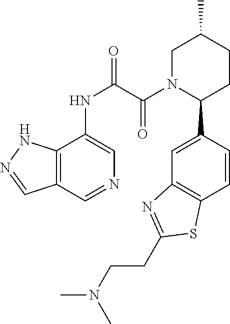 [SMILES: C[C@@H]1CC[C@@H](c2ccc3sc(CCN(C)C)nc3c2)N(C(=O)C(=O)Nc2cncc3cn[nH]c23)C1] | 70 | (abs) | c | cc | ccc | C | C* |
| 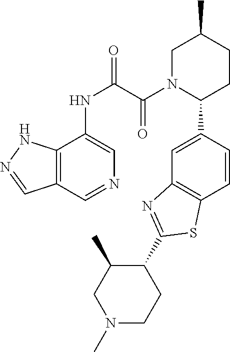 [SMILES: C[C@H]1CC[C@H](c2ccc3sc([C@@H]4CCN(C)C[C@H]4C)nc3c2)N(C(=O)C(=O)Nc2cncc3cn[nH]c23)C1] | 71 | (abs) core piperidine, (or) R³ piperidine | b | aa | aaa | C | A* |
| 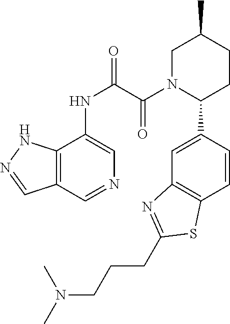 [SMILES: C[C@H]1CC[C@H](c2ccc3sc(CCCN(C)C)nc3c2)N(C(=O)C(=O)Nc2cncc3cn[nH]c23)C1] | 72 | (abs) | c | aa | aaa | A | A* |
| 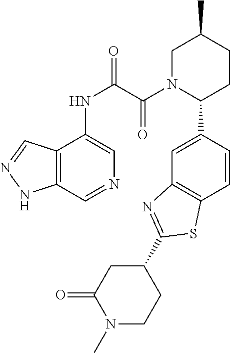 [SMILES: C[C@H]1CC[C@H](c2ccc3sc([C@@H]4CCN(C)C(=O)C4)nc3c2)N(C(=O)C(=O)Nc2cncc3[nH]ncc23)C1] | 73 | (abs) piperidine, (or) R³ piperidinone | c | bb | ccc | A | B* |
| 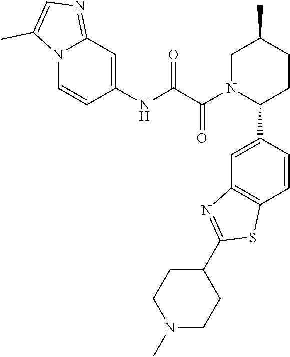 [SMILES: Cc1cnc2cc(NC(=O)C(=O)N3C[C@@H](C)CC[C@@H]3c3ccc4sc(C5CCN(C)CC5)nc4c3)ccn12] | 74 | (abs) | c | aa | aaa | A | A* |
| 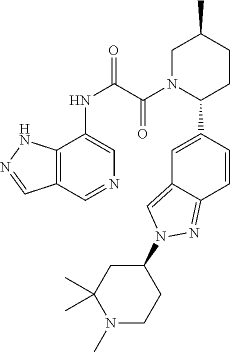 [SMILES: C[C@H]1CC[C@H](c2ccc3nn([C@H]4CCN(C)C(C)(C)C4)cc3c2)N(C(=O)C(=O)Nc2cncc3cn[nH]c23)C1] | 75 | (abs) core piperidine, (or) R³ piperidine | b | aa | aaa | C | A* |
| 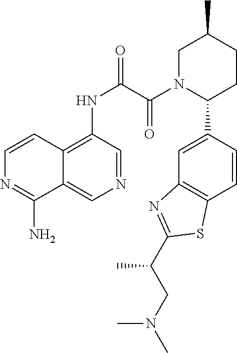 [SMILES: C[C@H]1CC[C@H](c2ccc3sc([C@@H](C)CN(C)C)nc3c2)N(C(=O)C(=O)Nc2cncc3c(N)nccc23)C1] | 76 | (abs) piperidine, (or) R³ side chain | c | bb | aaa | A | A* |
| 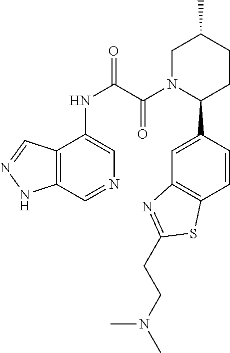 [SMILES: C[C@@H]1CC[C@@H](c2ccc3sc(CCN(C)C)nc3c2)N(C(=O)C(=O)Nc2cncc3[nH]ncc23)C1] | 77 | (abs) | c | cc | ccc | C | C* |
| 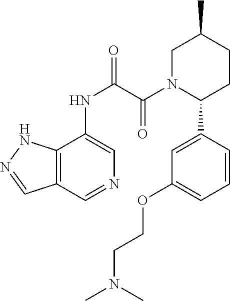 [SMILES: C[C@H]1CC[C@H](c2cccc(OCCN(C)C)c2)N(C(=O)C(=O)Nc2cncc3cn[nH]c23)C1] | 78 | (abs) | c | aa | aaa | B | B* |
| 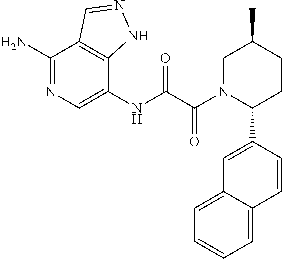 [SMILES: C[C@H]1CC[C@H](c2ccc3ccccc3c2)N(C(=O)C(=O)Nc2cnc(N)c3cn[nH]c23)C1] | 79 | (abs) | b | aa | aaa | A | A* |
| 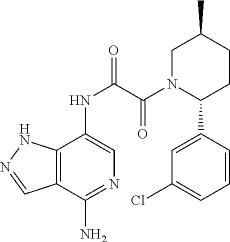 [SMILES: C[C@H]1CC[C@H](c2cccc(Cl)c2)N(C(=O)C(=O)Nc2cnc(N)c3cn[nH]c23)C1] | 80 | (abs) | b | aa | aaa | B | A* |
| 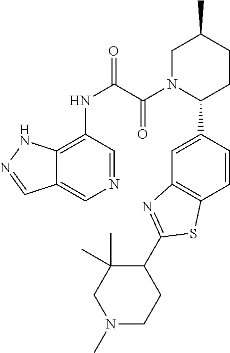 [SMILES: C[C@H]1CC[C@H](c2ccc3sc(C4CCN(C)CC4(C)C)nc3c2)N(C(=O)C(=O)Nc2cncc3cn[nH]c23)C1] | 81 | (abs) core piperidine | b | aa | aaa | B | A* |
| 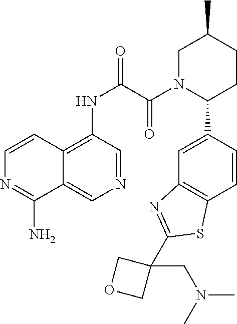 [SMILES: C[C@H]1CC[C@H](c2ccc3sc(C4(CN(C)C)COC4)nc3c2)N(C(=O)C(=O)Nc2cncc3c(N)nccc23)C1] | 82 | (abs) | c | bb | ccc | A | B* |
| 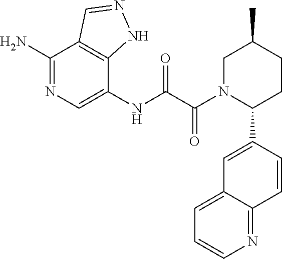 [SMILES: C[C@H]1CC[C@H](c2ccc3ncccc3c2)N(C(=O)C(=O)Nc2cnc(N)c3cn[nH]c23)C1] | 83 | (abs) | b | aa | aaa | B | A* |
| 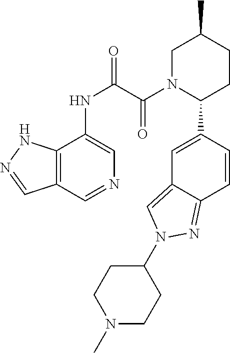 [SMILES: C[C@H]1CC[C@H](c2ccc3nn(C4CCN(C)CC4)cc3c2)N(C(=O)C(=O)Nc2cncc3cn[nH]c23)C1] | 84 | (abs) | c | aa | aaa | B | A* |
| 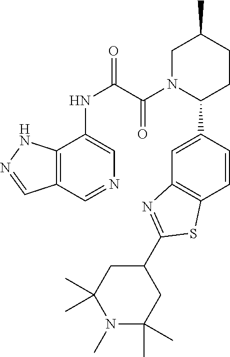 [SMILES: C[C@H]1CC[C@H](c2ccc3sc(C4CC(C)(C)N(C)C(C)(C)C4)nc3c2)N(C(=O)C(=O)Nc2cncc3cn[nH]c23)C1] | 85 | (abs) | b | | aaa | | A* |
| 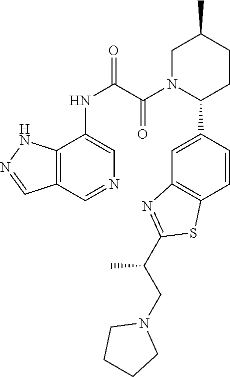 [SMILES: C[C@H]1CC[C@H](c2ccc3sc([C@@H](C)CN4CCCC4)nc3c2)N(C(=O)C(=O)Nc2cncc3cn[nH]c23)C1] | 86 | (abs) core piperidine, (or) R³ side chain | c | aa | aaa | B | A* |
| 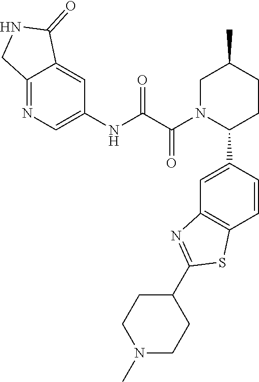 [SMILES: C[C@H]1CC[C@H](c2ccc3sc(C4CCN(C)CC4)nc3c2)N(C(=O)C(=O)Nc2cnc3c(c2)C(=O)NC3)C1] | 87 | (abs) | c | bb | ccc | A | B* |
| 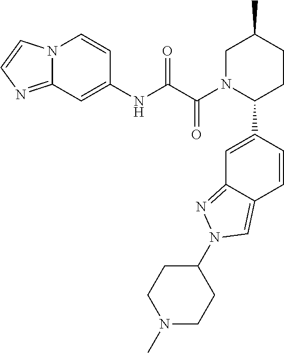 [SMILES: C[C@H]1CC[C@H](c2ccc3cn(C4CCN(C)CC4)nc3c2)N(C(=O)C(=O)Nc2ccn3ccnc3c2)C1] | 88 | (abs) | b | aa | aaa | B | A* |
| 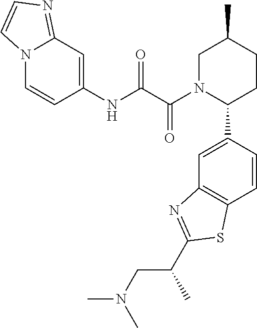 [SMILES: C[C@H]1CC[C@H](c2ccc3sc([C@H](C)CN(C)C)nc3c2)N(C(=O)C(=O)Nc2ccn3ccnc3c2)C1] | 89 | (abs) piperidine, (or) R³ side chain | c | aa | aaa | B | A* |
| 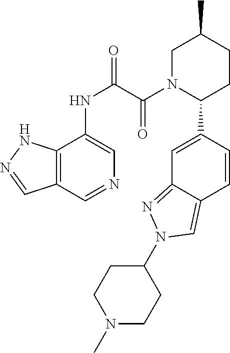 [SMILES: C[C@H]1CC[C@H](c2ccc3cn(C4CCN(C)CC4)nc3c2)N(C(=O)C(=O)Nc2cncc3cn[nH]c23)C1] | 90 | (abs) | b | aa | aaa | B | A* |
| 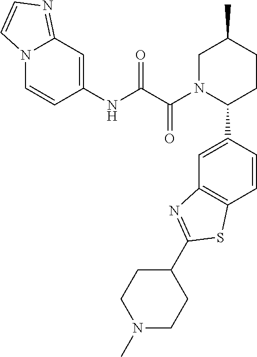 [SMILES: C[C@H]1CC[C@H](c2ccc3sc(C4CCN(C)CC4)nc3c2)N(C(=O)C(=O)Nc2ccn3ccnc3c2)C1] | 91 | (abs) | b | aa | aaa | B | A* |
| 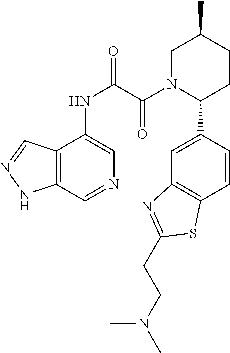 [SMILES: C[C@H]1CC[C@H](c2ccc3sc(CCN(C)C)nc3c2)N(C(=O)C(=O)Nc2cncc3[nH]ncc23)C1] | 92 | (abs) | c | aa | ccc | B | A* |
| 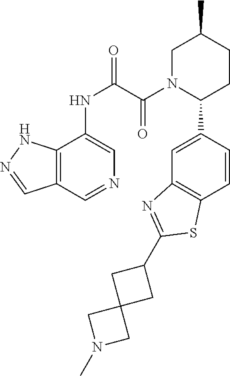 [SMILES: C[C@H]1CC[C@H](c2ccc3sc(C4CC5(C4)CN(C)C5)nc3c2)N(C(=O)C(=O)Nc2cncc3cn[nH]c23)C1] | 93 | (abs) | b | aa | aaa | C | A* |
| 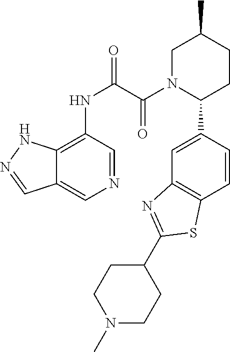 [SMILES: C[C@H]1CC[C@H](c2ccc3sc(C4CCN(C)CC4)nc3c2)N(C(=O)C(=O)Nc2cncc3cn[nH]c23)C1] | 94 | (abs) | b | aa | aaa | B | A* |
| 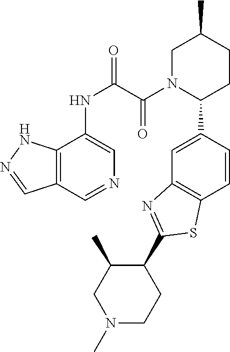 [SMILES: C[C@H]1CC[C@H](c2ccc3sc([C@H]4CCN(C)C[C@H]4C)nc3c2)N(C(=O)C(=O)Nc2cncc3cn[nH]c23)C1] | 95 | (abs) core piperidine (or) R³ piperidine | b | aa | aaa | B | A* |
| 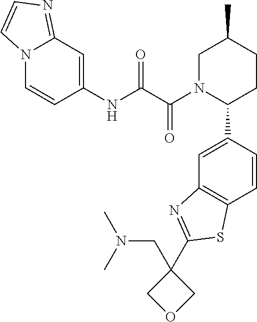 [SMILES: C[C@H]1CC[C@H](c2ccc3sc(C4(CN(C)C)COC4)nc3c2)N(C(=O)C(=O)Nc2ccn3ccnc3c2)C1] | 96 | (abs) | c | bb | bbb | A | B* |
| 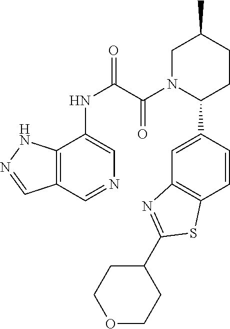 [SMILES: C[C@H]1CC[C@H](c2ccc3sc(C4CCOCC4)nc3c2)N(C(=O)C(=O)Nc2cncc3cn[nH]c23)C1] | 97 | (abs) | c | aa | bbb | A | A* |
| 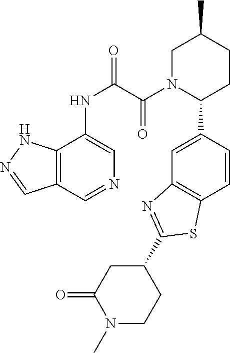 [SMILES: C[C@H]1CC[C@H](c2ccc3sc([C@@H]4CCN(C)C(=O)C4)nc3c2)N(C(=O)C(=O)Nc2cncc3cn[nH]c23)C1] | 98 | (abs) piperidine, (or) R³ piperidinone | c | bb | bbb | B | B* |
| 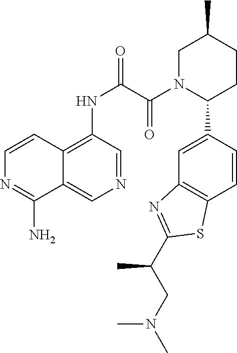 [SMILES: C[C@H]1CC[C@H](c2ccc3sc([C@H](C)CN(C)C)nc3c2)N(C(=O)C(=O)Nc2cncc3c(N)nccc23)C1] | 99 | (abs) piperidine, (or) R³ side chain | c | aa | aaa | A | A* |
| 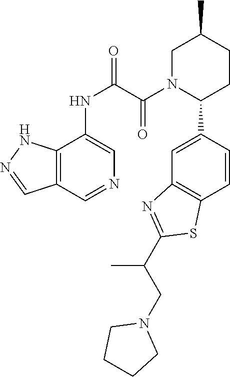 [SMILES: CC(CN1CCCC1)c1nc2cc([C@H]3CC[C@H](C)CN3C(=O)C(=O)Nc3cncc4cn[nH]c34)ccc2s1] | 100 | (abs) core piperidine | c | aa | aaa | A | A* |
| 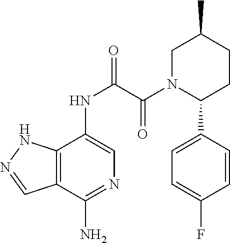 [SMILES: C[C@H]1CC[C@H](c2ccc(F)cc2)N(C(=O)C(=O)Nc2cnc(N)c3cn[nH]c23)C1] | 101 | (abs) | b | aa | aaa | B | A* |
| 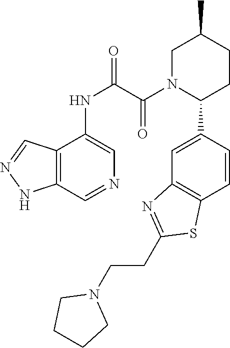 [SMILES: C[C@H]1CC[C@H](c2ccc3sc(CCN4CCCC4)nc3c2)N(C(=O)C(=O)Nc2cncc3[nH]ncc23)C1] | 102 | (abs) | c | aa | aaa | A | A* |
| 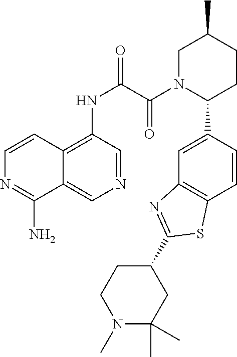 [SMILES: C[C@H]1CC[C@H](c2ccc3sc([C@H]4CCN(C)C(C)(C)C4)nc3c2)N(C(=O)C(=O)Nc2cncc3c(N)nccc23)C1] | 103 | (abs) core piperidine, (or) R³ piperidine | b | aa | aaa | B | A* |
| 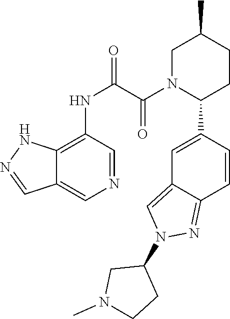 [SMILES: C[C@H]1CC[C@H](c2ccc3nn([C@H]4CCN(C)C4)cc3c2)N(C(=O)C(=O)Nc2cncc3cn[nH]c23)C1] | 104 | (abs) | c | bb | bbb | A | B* |
| 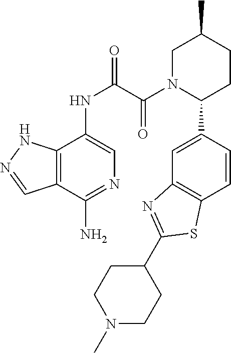 [SMILES: C[C@H]1CC[C@H](c2ccc3sc(C4CCN(C)CC4)nc3c2)N(C(=O)C(=O)Nc2cnc(N)c3cn[nH]c23)C1] | 105 | (abs) | a | aa | aaa | C | A* |
| 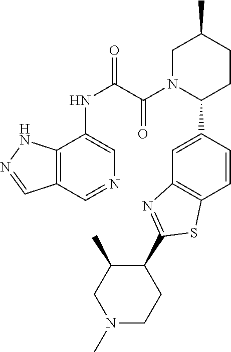 [SMILES: C[C@H]1CC[C@H](c2ccc3sc([C@H]4CCN(C)C[C@H]4C)nc3c2)N(C(=O)C(=O)Nc2cncc3cn[nH]c23)C1] | 106 | (abs) core piperidine, (or) R³ piperidine | b | aa | aaa | C | A* |
| 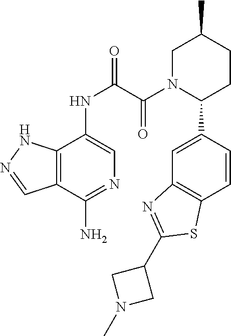 [SMILES: C[C@H]1CC[C@H](c2ccc3sc(C4CN(C)C4)nc3c2)N(C(=O)C(=O)Nc2cnc(N)c3cn[nH]c23)C1] | 107 | (abs) | b | aa | aaa | C | A* |
| 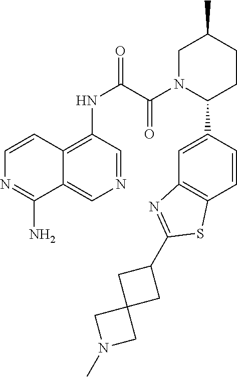 [SMILES: C[C@H]1CC[C@H](c2ccc3sc(C4CC5(C4)CN(C)C5)nc3c2)N(C(=O)C(=O)Nc2cncc3c(N)nccc23)C1] | 108 | (abs) | c | aa | bbb | B | A* |
| 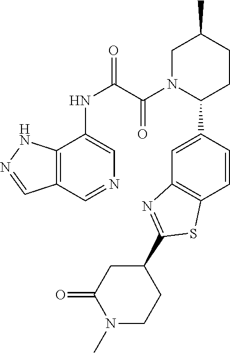 [SMILES: C[C@H]1CC[C@H](c2ccc3sc([C@H]4CCN(C)C(=O)C4)nc3c2)N(C(=O)C(=O)Nc2cncc3cn[nH]c23)C1] | 109 | (abs) piperidine, (or) R³ piperidinone | c | bb | ccc | B | B* |
| 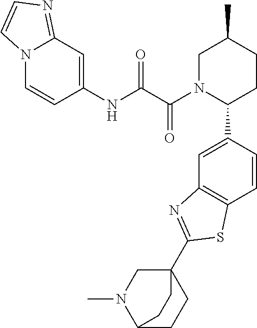 [SMILES: C[C@H]1CC[C@H](c2ccc3sc(C45CCC(CC4)N(C)C5)nc3c2)N(C(=O)C(=O)Nc2ccn3ccnc3c2)C1] | 110 | (abs) | b | aa | aaa | A | A* |
| 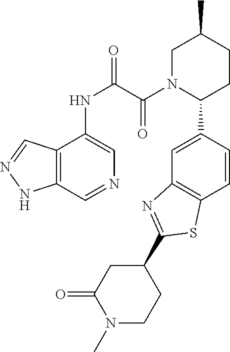 [SMILES: C[C@H]1CC[C@H](c2ccc3sc([C@H]4CCN(C)C(=O)C4)nc3c2)N(C(=O)C(=O)Nc2cncc3[nH]ncc23)C1] | 111 | (abs) piperidine, (or) R³ piperidinone | c | bb | ccc | A | B* |
| 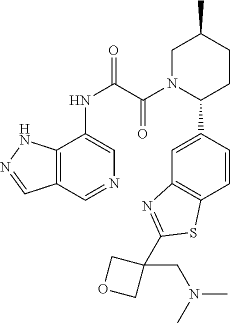 [SMILES: C[C@H]1CC[C@H](c2ccc3sc(C4(CN(C)C)COC4)nc3c2)N(C(=O)C(=O)Nc2cncc3cn[nH]c23)C1] | 112 | (abs) | c | aa | aaa | B | A* |
| 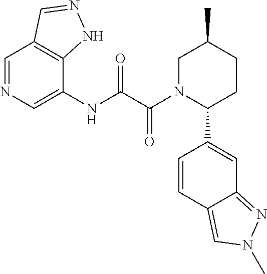 [SMILES: C[C@H]1CC[C@H](c2ccc3cn(C)nc3c2)N(C(=O)C(=O)Nc2cncc3cn[nH]c23)C1] | 113 | | | | | | |
| 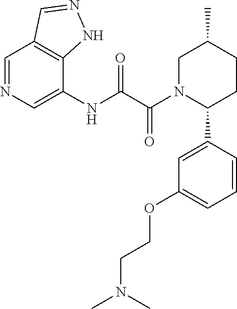 [SMILES: C[C@@H]1CC[C@H](c2cccc(OCCN(C)C)c2)N(C(=O)C(=O)Nc2cncc3cn[nH]c23)C1] | 114 | | | | | | |
| 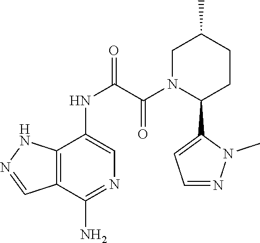 [SMILES: C[C@@H]1CC[C@@H](c2ccnn2C)N(C(=O)C(=O)Nc2cnc(N)c3cn[nH]c23)C1] | 115 | (abs) | c | aa | aaa | A | A* |
| 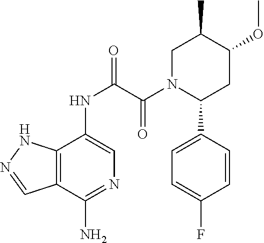 [SMILES: CO[C@@H]1C[C@H](c2ccc(F)cc2)N(C(=O)C(=O)Nc2cnc(N)c3cn[nH]c23)C[C@H]1C] | 116 | (or) | c | aa | aaa | A | A* |
| 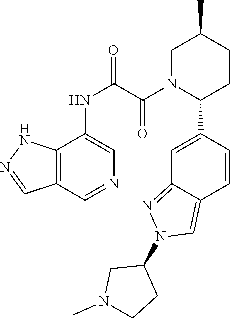 [SMILES: C[C@H]1CC[C@H](c2ccc3cn([C@H]4CCN(C)C4)nc3c2)N(C(=O)C(=O)Nc2cncc3cn[nH]c23)C1] | 117 | (abs) | b | aa | aaa | A | A* |
| 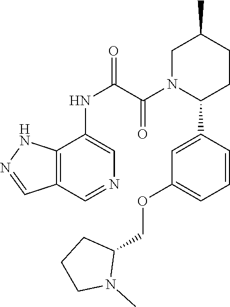 [SMILES: C[C@H]1CC[C@H](c2cccc(OC[C@H]3CCCN3C)c2)N(C(=O)C(=O)Nc2cncc3cn[nH]c23)C1] | 118 | (abs) | c | bb | ccc | A | B* |
| 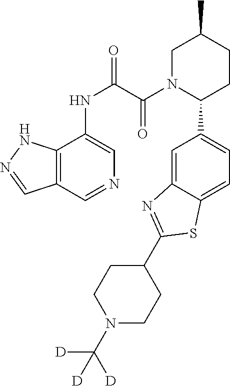 [SMILES: [2H]C([2H])([2H])N1CCC(c2nc3cc([C@H]4CC[C@H](C)CN4C(=O)C(=O)Nc4cncc5cn[nH]c45)ccc3s2)CC1] | 119 | (abs) | b | aa | aaa | B | A* |
| 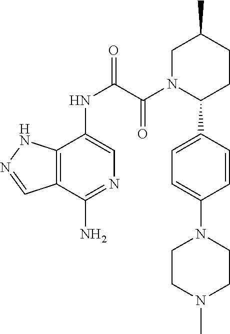 [SMILES: C[C@H]1CC[C@H](c2ccc(N3CCN(C)CC3)cc2)N(C(=O)C(=O)Nc2cnc(N)c3cn[nH]c23)C1] | 120 | (abs) | c | bb | bbb | B | |
| 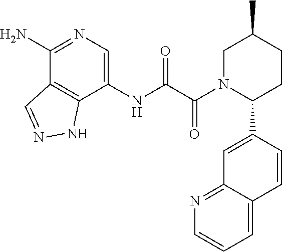 [SMILES: C[C@H]1CC[C@H](c2ccc3cccnc3c2)N(C(=O)C(=O)Nc2cnc(N)c3cn[nH]c23)C1] | 121 | (abs) | b | aa | aaa | A | A* |
| 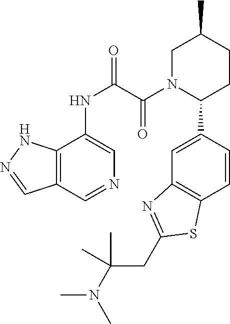 [SMILES: C[C@H]1CC[C@H](c2ccc3sc(CC(C)(C)N(C)C)nc3c2)N(C(=O)C(=O)Nc2cncc3cn[nH]c23)C1] | 122 | (abs) | c | aa | aaa | A | A* |
| 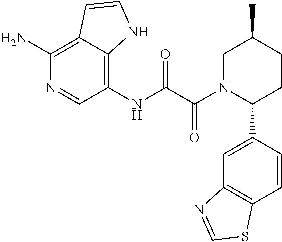 [SMILES: C[C@H]1CC[C@H](c2ccc3scnc3c2)N(C(=O)C(=O)Nc2cnc(N)c3cc[nH]c23)C1] | 123 | (abs) | c | aa | aaa | C | B* |
| 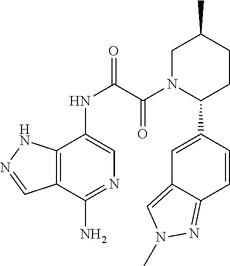 [SMILES: C[C@H]1CC[C@H](c2ccc3nn(C)cc3c2)N(C(=O)C(=O)Nc2cnc(N)c3cn[nH]c23)C1] | 124 | (abs) | c | aa | aaa | B | A* |
| 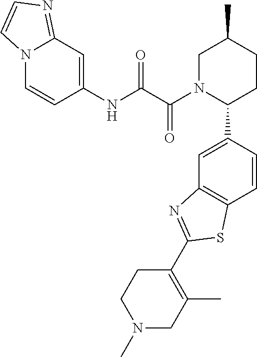 [SMILES: CC1=C(c2nc3cc([C@H]4CC[C@H](C)CN4C(=O)C(=O)Nc4ccn5ccnc5c4)ccc3s2)CCN(C)C1] | 125 | (abs) | c | aa | aaa | A | A* |
| 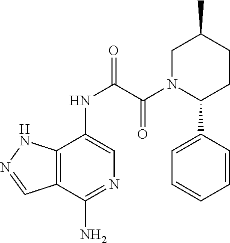 [SMILES: C[C@H]1CC[C@H](c2ccccc2)N(C(=O)C(=O)Nc2cnc(N)c3cn[nH]c23)C1] | 126 | (abs) | b | aa | aaa | B | A* |
| 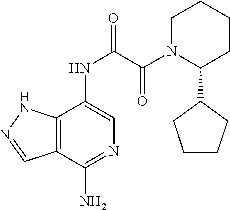 [SMILES: Nc1ncc(NC(=O)C(=O)N2CCCC[C@@H]2C2CCCC2)c2[nH]ncc12] | 127 | (abs) | c | aa | aaa | A | A* |
| 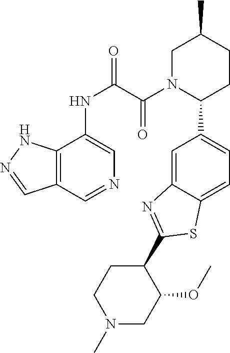 [SMILES: CO[C@@H]1CN(C)CC[C@H]1c1nc2cc([C@H]3CC[C@H](C)CN3C(=O)C(=O)Nc3cncc4cn[nH]c34)ccc2s1] | 128 | (abs) core piperidine, (or) R³ piperidine | c | aa | bbb | A | A* |
| 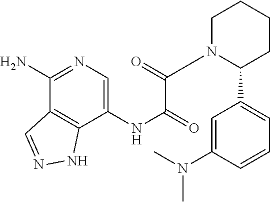 [SMILES: CN(C)c1cccc([C@H]2CCCCN2C(=O)C(=O)Nc2cnc(N)c3cn[nH]c23)c1] | 129 | (abs) | c | aa | aaa | B | A* |
| 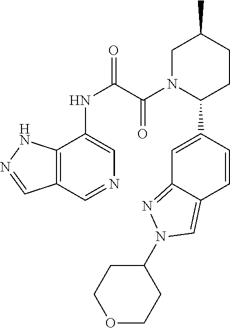 [SMILES: C[C@H]1CC[C@H](c2ccc3cn(C4CCOCC4)nc3c2)N(C(=O)C(=O)Nc2cncc3cn[nH]c23)C1] | 130 | (abs) | c | aa | bbb | A | A* |
| 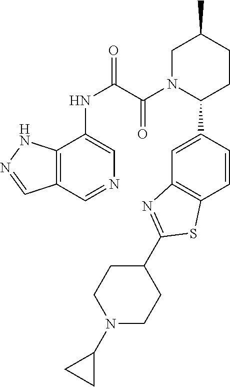 [SMILES: C[C@H]1CC[C@H](c2ccc3sc(C4CCN(C5CC5)CC4)nc3c2)N(C(=O)C(=O)Nc2cncc3cn[nH]c23)C1] | 131 | (abs) | b | aa | aaa | C | A* |
| 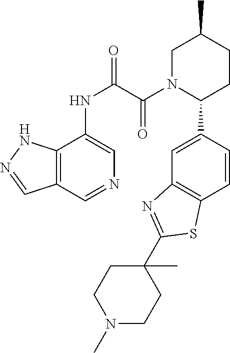 [SMILES: C[C@H]1CC[C@H](c2ccc3sc(C4(C)CCN(C)CC4)nc3c2)N(C(=O)C(=O)Nc2cncc3cn[nH]c23)C1] | 132 | (abs) | b | aa | aaa | B | A* |
| 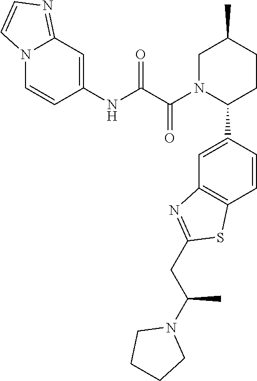 [SMILES: C[C@H]1CC[C@H](c2ccc3sc(C[C@@H](C)N4CCCC4)nc3c2)N(C(=O)C(=O)Nc2ccn3ccnc3c2)C1] | 133 | (abs) core piperidine, (and) R³ chain | c | aa | bbb | A | A* |
| 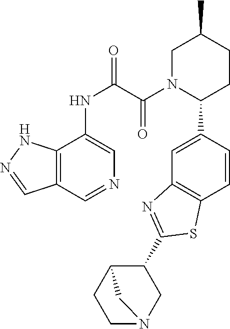 [SMILES: C[C@H]1CC[C@H](c2ccc3sc([C@@H]4CN5CC[C@@H]4C5)nc3c2)N(C(=O)C(=O)Nc2cncc3cn[nH]c23)C1] | 134 | (abs) piperidine, (rel) bicycle | b | aa | aaa | C | A* |
| 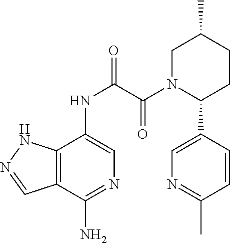 [SMILES: Cc1ccc([C@H]2CC[C@@H](C)CN2C(=O)C(=O)Nc2cnc(N)c3cn[nH]c23)cn1] | 135 | (abs) | c | cc | ccc | C | C* |
| 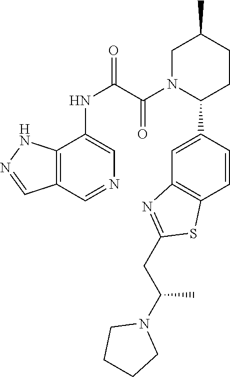 [SMILES: C[C@H]1CC[C@H](c2ccc3sc(C[C@H](C)N4CCCC4)nc3c2)N(C(=O)C(=O)Nc2cncc3cn[nH]c23)C1] | 136 | (abs) | c | aa | aaa | A | A* |
| 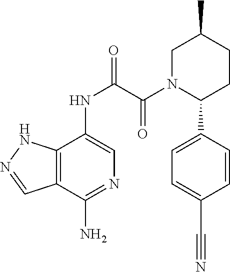 [SMILES: C[C@H]1CC[C@H](c2ccc(C#N)cc2)N(C(=O)C(=O)Nc2cnc(N)c3cn[nH]c23)C1] | 137 | (abs) | b | aa | aaa | A | |
| 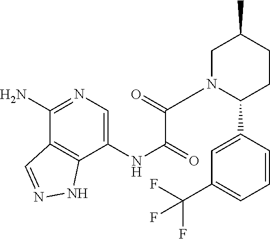 [SMILES: C[C@H]1CC[C@H](c2cccc(C(F)(F)F)c2)N(C(=O)C(=O)Nc2cnc(N)c3cn[nH]c23)C1] | 138 | (abs) | c | bb | aaa | A | A* |
| 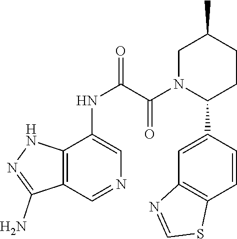 [SMILES: C[C@H]1CC[C@H](c2ccc3scnc3c2)N(C(=O)C(=O)Nc2cncc3c(N)n[nH]c23)C1] | 139 | (abs) | c | bb | ccc | A | B* |
| 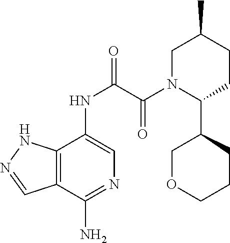 [SMILES: C[C@H]1CC[C@H]([C@H]2CCCOC2)N(C(=O)C(=O)Nc2cnc(N)c3cn[nH]c23)C1] | 140 | (abs) core piperidine, (or) R³ tetrahydropyran | c | bb | ccc | A | B* |
| 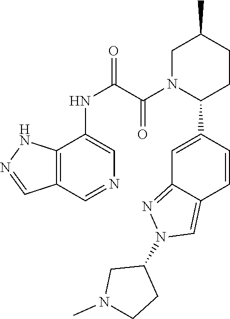 [SMILES: C[C@H]1CC[C@H](c2ccc3cn([C@@H]4CCN(C)C4)nc3c2)N(C(=O)C(=O)Nc2cncc3cn[nH]c23)C1] | 141 | (abs) | c | aa | aaa | A | A* |
| 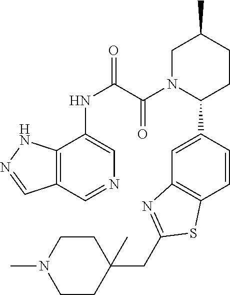 [SMILES: C[C@H]1CC[C@H](c2ccc3sc(CC4(C)CCN(C)CC4)nc3c2)N(C(=O)C(=O)Nc2cncc3cn[nH]c23)C1] | 142 | (abs) | b | aa | aaa | B | A* |
| 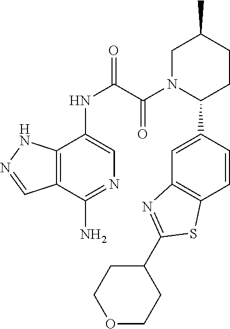 [SMILES: C[C@H]1CC[C@H](c2ccc3sc(C4CCOCC4)nc3c2)N(C(=O)C(=O)Nc2cnc(N)c3cn[nH]c23)C1] | 143 | (abs) | b | aa | aaa | C | A* |
| 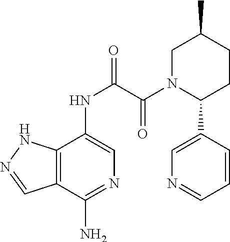 [SMILES: C[C@H]1CC[C@H](c2cccnc2)N(C(=O)C(=O)Nc2cnc(N)c3cn[nH]c23)C1] | 144 | (abs) | c | aa | aaa | A | |
| 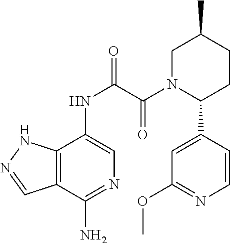 [SMILES: COc1cc([C@H]2CC[C@H](C)CN2C(=O)C(=O)Nc2cnc(N)c3cn[nH]c23)ccn1] | 145 | (abs) | b | aa | aaa | B | A* |
| 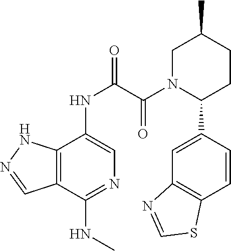 [SMILES: CNc1ncc(NC(=O)C(=O)N2C[C@@H](C)CC[C@@H]2c2ccc3scnc3c2)c2[nH]ncc12] | 146 | (abs) | c | bb | bbb | A | B* |
| 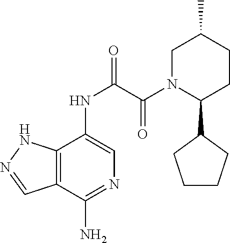 [SMILES: C[C@@H]1CC[C@@H](C2CCCC2)N(C(=O)C(=O)Nc2cnc(N)c3cn[nH]c23)C1] | 147 | (and) | c | aa | aaa | A | A* |
| 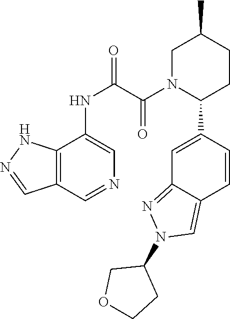 [SMILES: C[C@H]1CC[C@H](c2ccc3cn([C@H]4CCOC4)nc3c2)N(C(=O)C(=O)Nc2cncc3cn[nH]c23)C1] | 148 | (abs) core piperidine, (and) R³ THF | c | bb | bbb | B | B* |
| 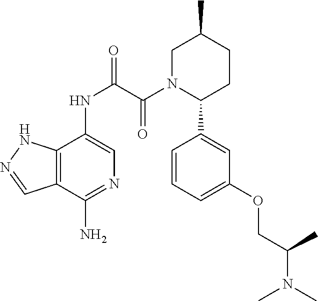 [SMILES: C[C@H]1CC[C@H](c2cccc(OC[C@@H](C)N(C)C)c2)N(C(=O)C(=O)Nc2cnc(N)c3cn[nH]c23)C1] | 149 | (abs) | b | aa | aaa | C | A* |
| 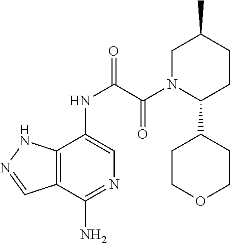 [SMILES: C[C@H]1CC[C@H](C2CCOCC2)N(C(=O)C(=O)Nc2cnc(N)c3cn[nH]c23)C1] | 150 | (abs) | c | bb | ccc | A | |
| 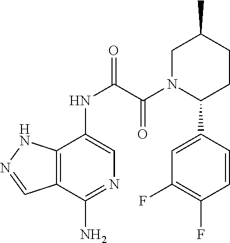 [SMILES: C[C@H]1CC[C@H](c2ccc(F)c(F)c2)N(C(=O)C(=O)Nc2cnc(N)c3cn[nH]c23)C1] | 151 | (abs) | c | aa | aaa | A | A* |
| 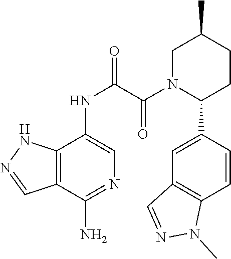 [SMILES: C[C@H]1CC[C@H](c2ccc3c(cnn3C)c2)N(C(=O)C(=O)Nc2cnc(N)c3cn[nH]c23)C1] | 152 | (abs) | b | aa | aaa | B | A* |
| 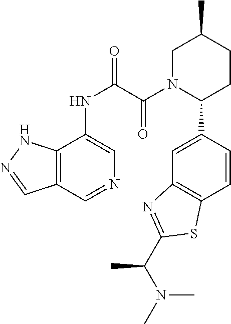 [SMILES: C[C@H]1CC[C@H](c2ccc3sc([C@H](C)N(C)C)nc3c2)N(C(=O)C(=O)Nc2cncc3cn[nH]c23)C1] | 153 | (abs) core piperidine, (or) R³ chain | c | aa | aaa | B | A* |
| 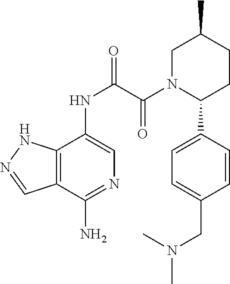 [SMILES: C[C@H]1CC[C@H](c2ccc(CN(C)C)cc2)N(C(=O)C(=O)Nc2cnc(N)c3cn[nH]c23)C1] | 154 | (abs) | c | aa | aaa | B | A* |
| 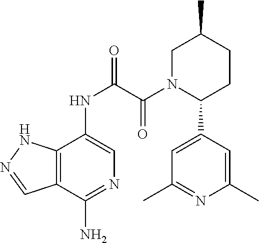 [SMILES: Cc1cc([C@H]2CC[C@H](C)CN2C(=O)C(=O)Nc2cnc(N)c3cn[nH]c23)cc(C)n1] | 155 | (abs) | c | bb | bbb | C | A* |
| 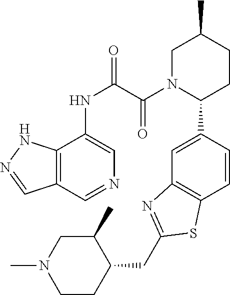 [SMILES: C[C@H]1CC[C@H](c2ccc3sc(C[C@@H]4CCN(C)C[C@H]4C)nc3c2)N(C(=O)C(=O)Nc2cncc3cn[nH]c23)C1] | 156 | (abs) piperidine core, (or) R³ piperidine | b | aa | aaa | B | A* |
| 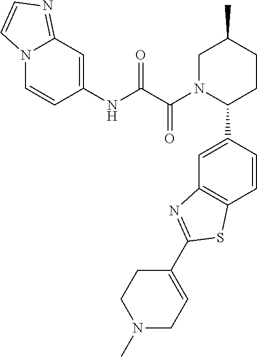 [SMILES: C[C@H]1CC[C@H](c2ccc3sc(C4=CCN(C)CC4)nc3c2)N(C(=O)C(=O)Nc2ccn3ccnc3c2)C1] | 157 | (abs) | c | aa | aaa | A | A* |
| 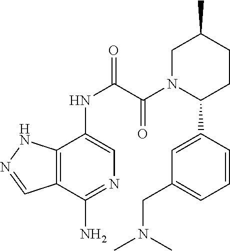 [SMILES: C[C@H]1CC[C@H](c2cccc(CN(C)C)c2)N(C(=O)C(=O)Nc2cnc(N)c3cn[nH]c23)C1] | 158 | (abs) | c | aa | aaa | B | A* |
| 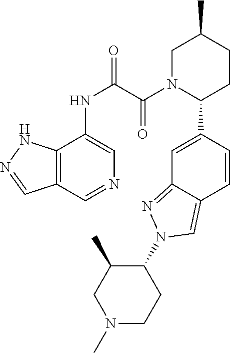 [SMILES: C[C@H]1CC[C@H](c2ccc3cn([C@@H]4CCN(C)C[C@H]4C)nc3c2)N(C(=O)C(=O)Nc2cncc3cn[nH]c23)C1] | 159 | (abs) piperidine core, (or) R³ piperidine | c | aa | aaa | B | A* |
| 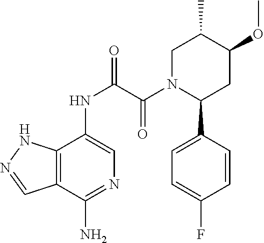 [SMILES: CO[C@H]1C[C@@H](c2ccc(F)cc2)N(C(=O)C(=O)Nc2cnc(N)c3cn[nH]c23)C[C@@H]1C] | 160 | (or) | c | cc | ccc | C | B* |
| 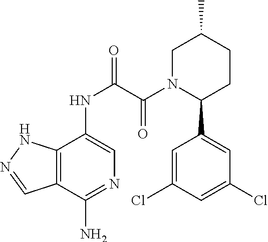 [SMILES: C[C@@H]1CC[C@@H](c2cc(Cl)cc(Cl)c2)N(C(=O)C(=O)Nc2cnc(N)c3cn[nH]c23)C1] | 161 | (and) | c | aa | aaa | A | A* |
| 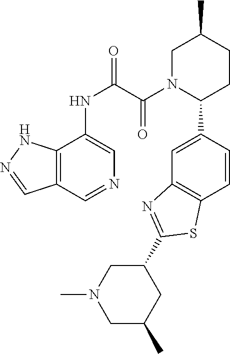 [SMILES: C[C@@H]1C[C@@H](c2nc3cc([C@H]4CC[C@H](C)CN4C(=O)C(=O)Nc4cncc5cn[nH]c45)ccc3s2)CN(C)C1] | 162 | (abs) core piperidine, (or) R³ piperidine | b | aa | aaa | B | A* |
| 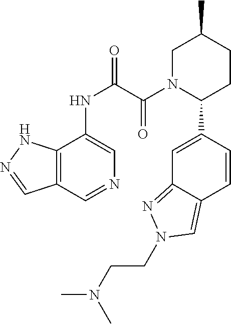 [SMILES: C[C@H]1CC[C@H](c2ccc3cn(CCN(C)C)nc3c2)N(C(=O)C(=O)Nc2cncc3cn[nH]c23)C1] | 163 | (abs) | c | aa | aaa | A | A* |
| 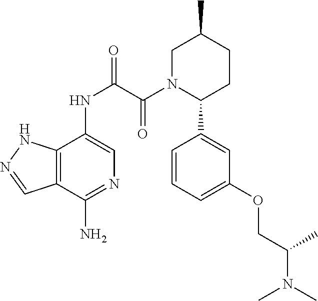 [SMILES: C[C@H]1CC[C@H](c2cccc(OC[C@H](C)N(C)C)c2)N(C(=O)C(=O)Nc2cnc(N)c3cn[nH]c23)C1] | 164 | (abs) | b | aa | aaa | C | A* |
| 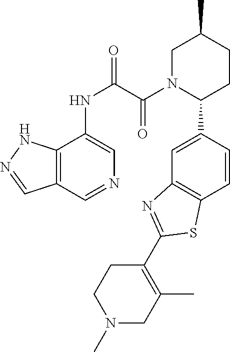 [SMILES: CC1=C(c2nc3cc([C@H]4CC[C@H](C)CN4C(=O)C(=O)Nc4cncc5cn[nH]c45)ccc3s2)CCN(C)C1] | 165 | (abs) | c | aa | aaa | A | A* |
| 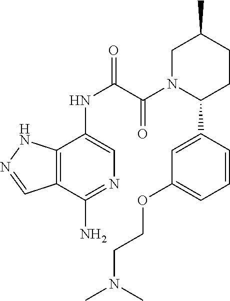 [SMILES: C[C@H]1CC[C@H](c2cccc(OCCN(C)C)c2)N(C(=O)C(=O)Nc2cnc(N)c3cn[nH]c23)C1] | 166 | (abs) | b | aa | aaa | C | A* |
| 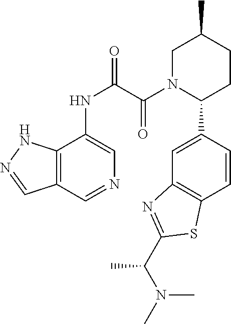 [SMILES: C[C@H]1CC[C@H](c2ccc3sc([C@@H](C)N(C)C)nc3c2)N(C(=O)C(=O)Nc2cncc3cn[nH]c23)C1] | 167 | (abs) core piperidine, (or) R³ chain | c | bb | aaa | A | A* |
| 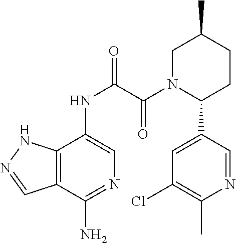 [SMILES: Cc1ncc([C@H]2CC[C@H](C)CN2C(=O)C(=O)Nc2cnc(N)c3cn[nH]c23)cc1Cl] | 168 | (abs) | c | aa | aaa | C | A* |
| 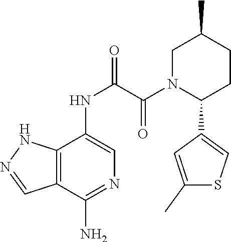 [SMILES: Cc1cc([C@H]2CC[C@H](C)CN2C(=O)C(=O)Nc2cnc(N)c3cn[nH]c23)cs1] | 169 | (abs) | c | aa | aaa | B | A* |
| 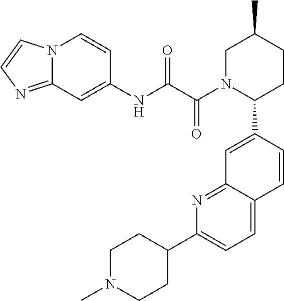 [SMILES: C[C@H]1CC[C@H](c2ccc3ccc(C4CCN(C)CC4)nc3c2)N(C(=O)C(=O)Nc2ccn3ccnc3c2)C1] | 170 | (abs) | c | bb | ccc | A | A* |
| 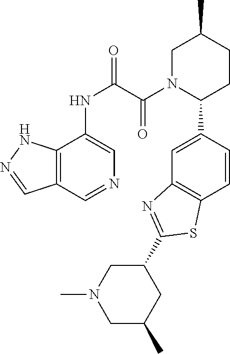 [SMILES: C[C@@H]1C[C@@H](c2nc3cc([C@H]4CC[C@H](C)CN4C(=O)C(=O)Nc4cncc5cn[nH]c45)ccc3s2)CN(C)C1] | 171 | (abs) core piperidine, (or) R³ piperidine | c | aa | aaa | B | A* |
| 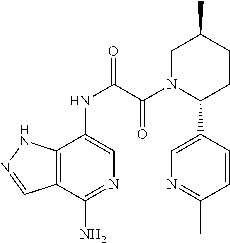 [SMILES: Cc1ccc([C@H]2CC[C@H](C)CN2C(=O)C(=O)Nc2cnc(N)c3cn[nH]c23)cn1] | 172 | (abs) | c | aa | aaa | A | A* |
| 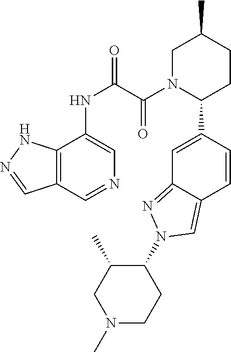 [SMILES: C[C@H]1CC[C@H](c2ccc3cn([C@@H]4CCN(C)C[C@@H]4C)nc3c2)N(C(=O)C(=O)Nc2cncc3cn[nH]c23)C1] | 173 | (abs) core piperidine, (or) R³ piperidine | c | aa | aaa | A | A* |
| 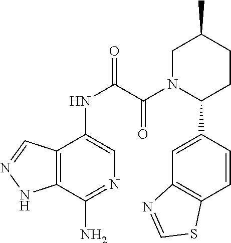 [SMILES: C[C@H]1CC[C@H](c2ccc3scnc3c2)N(C(=O)C(=O)Nc2cnc(N)c3[nH]ncc23)C1] | 174 | (abs) | b | aa | aaa | C | A* |
| 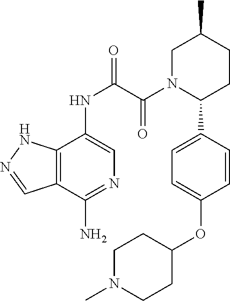 [SMILES: C[C@H]1CC[C@H](c2ccc(OC3CCN(C)CC3)cc2)N(C(=O)C(=O)Nc2cnc(N)c3cn[nH]c23)C1] | 175 | (abs) piperidine core, (and) R³ piperidine | c | bb | aaa | B | A* |
| 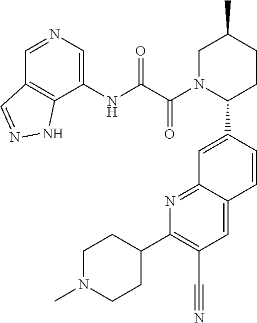 [SMILES: C[C@H]1CC[C@H](c2ccc3cc(C#N)c(C4CCN(C)CC4)nc3c2)N(C(=O)C(=O)Nc2cncc3cn[nH]c23)C1] | 176 | (abs) | c | aa | aaa | B | A* |
| 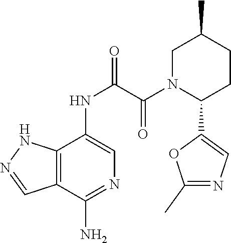 [SMILES: Cc1ncc([C@H]2CC[C@H](C)CN2C(=O)C(=O)Nc2cnc(N)c3cn[nH]c23)o1] | 177 | (abs) | c | bb | ccc | C | B* |
| 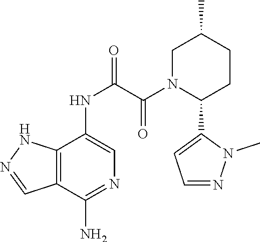 [SMILES: C[C@@H]1CC[C@H](c2ccnn2C)N(C(=O)C(=O)Nc2cnc(N)c3cn[nH]c23)C1] | 178 | (abs) | c | bb | ccc | C | B* |
| 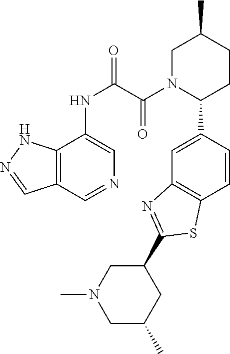 [SMILES: C[C@H]1C[C@H](c2nc3cc([C@H]4CC[C@H](C)CN4C(=O)C(=O)Nc4cncc5cn[nH]c45)ccc3s2)CN(C)C1] | 179 | (abs) core piperidine, (or) R³ piperidine | c | aa | aaa | B | A* |
| 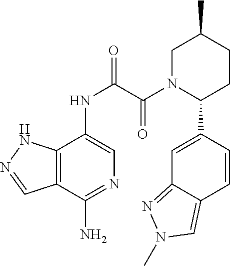 [SMILES: C[C@H]1CC[C@H](c2ccc3cn(C)nc3c2)N(C(=O)C(=O)Nc2cnc(N)c3cn[nH]c23)C1] | 180 | (abs) | b | aa | aaa | B | A* |
| 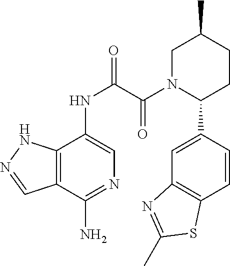 [SMILES: Cc1nc2cc([C@H]3CC[C@H](C)CN3C(=O)C(=O)Nc3cnc(N)c4cn[nH]c34)ccc2s1] | 181 | (abs) | b | aa | aaa | C | A* |
| 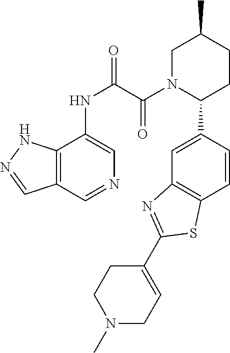 [SMILES: C[C@H]1CC[C@H](c2ccc3sc(C4=CCN(C)CC4)nc3c2)N(C(=O)C(=O)Nc2cncc3cn[nH]c23)C1] | 182 | (abs) | c | aa | aaa | A | A* |
| 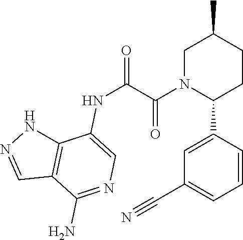 [SMILES: C[C@H]1CC[C@H](c2cccc(C#N)c2)N(C(=O)C(=O)Nc2cnc(N)c3cn[nH]c23)C1] | 183 | (abs) | b | aa | aaa | B | A* |
| 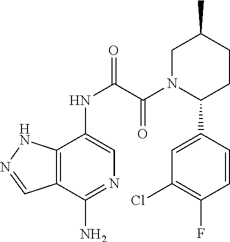 [SMILES: C[C@H]1CC[C@H](c2ccc(F)c(Cl)c2)N(C(=O)C(=O)Nc2cnc(N)c3cn[nH]c23)C1] | 184 | (and) | c | aa | aaa | A | A* |
| 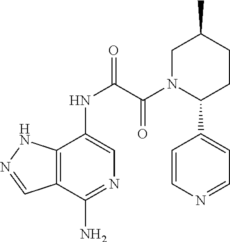 [SMILES: C[C@H]1CC[C@H](c2ccncc2)N(C(=O)C(=O)Nc2cnc(N)c3cn[nH]c23)C1] | 185 | (abs) | c | aa | aaa | B | |
| 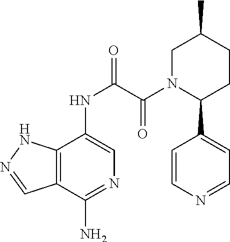 [SMILES: C[C@H]1CC[C@@H](c2ccncc2)N(C(=O)C(=O)Nc2cnc(N)c3cn[nH]c23)C1] | 186 | (abs) | | | | | |
| 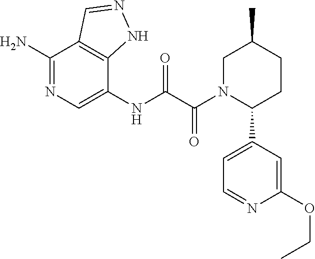 [SMILES: CCOc1cc([C@H]2CC[C@H](C)CN2C(=O)C(=O)Nc2cnc(N)c3cn[nH]c23)ccn1] | 187 | (abs) | b | aa | aaa | A | A* |
| 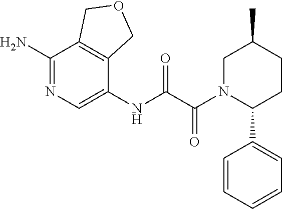 [SMILES: C[C@H]1CC[C@H](c2ccccc2)N(C(=O)C(=O)Nc2cnc(N)c3c2COC3)C1] | 188 | (abs) | c | bb | bbb | A | B* |
| 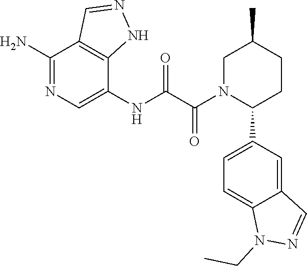 [SMILES: CCn1ncc2cc([C@H]3CC[C@H](C)CN3C(=O)C(=O)Nc3cnc(N)c4cn[nH]c34)ccc21] | 189 | (abs) | b | aa | aaa | C | A* |
| 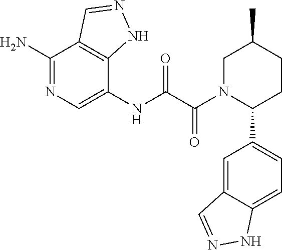 [SMILES: C[C@H]1CC[C@H](c2ccc3[nH]ncc3c2)N(C(=O)C(=O)Nc2cnc(N)c3cn[nH]c23)C1] | 190 | (abs) | b | aa | aaa | C | A* |
| 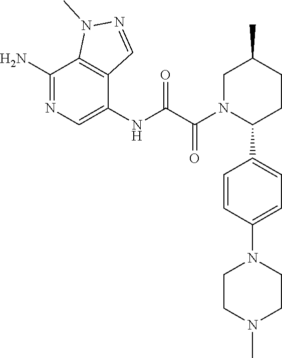 [SMILES: C[C@H]1CC[C@H](c2ccc(N3CCN(C)CC3)cc2)N(C(=O)C(=O)Nc2cnc(N)c3c2cnn3C)C1] | 191 | (abs) | c | aa | aaa | B | A* |
| 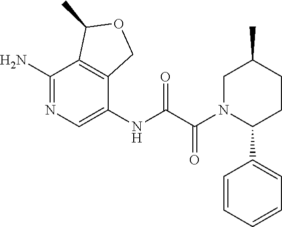 [SMILES: C[C@H]1CC[C@H](c2ccccc2)N(C(=O)C(=O)Nc2cnc(N)c3c2CO[C@@H]3C)C1] | 192 | (abs) on piperidine, (or) on dihydrofuran | c | bb | aaa | A | A* |
| 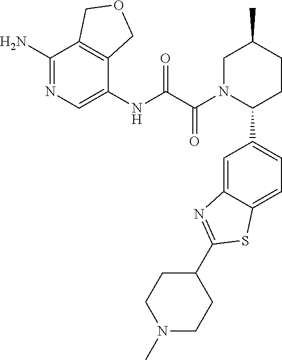 [SMILES: C[C@H]1CC[C@H](c2ccc3sc(C4CCN(C)CC4)nc3c2)N(C(=O)C(=O)Nc2cnc(N)c3c2COC3)C1] | 193 | (abs) | a | aa | aaa | C | A* |
| 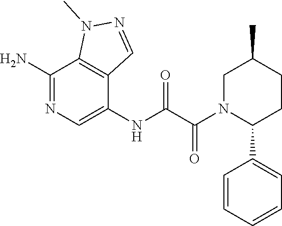 [SMILES: C[C@H]1CC[C@H](c2ccccc2)N(C(=O)C(=O)Nc2cnc(N)c3c2cnn3C)C1] | 194 | (abs) | c | aa | aaa | B | A* |
| 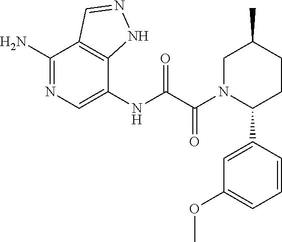 [SMILES: COc1cccc([C@H]2CC[C@H](C)CN2C(=O)C(=O)Nc2cnc(N)c3cn[nH]c23)c1] | 195 | (abs) | b | aa | aaa | B | A* |
| 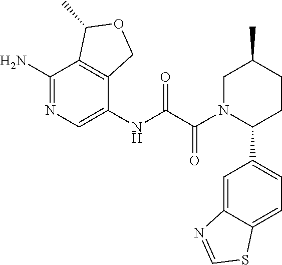 [SMILES: C[C@H]1CC[C@H](c2ccc3scnc3c2)N(C(=O)C(=O)Nc2cnc(N)c3c2CO[C@H]3C)C1] | 196 | (abs) on piperidine, (and) on dihydrofuran | c | bb | aaa | C | A* |
| 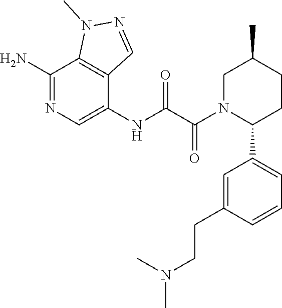 [SMILES: C[C@H]1CC[C@H](c2cccc(CCN(C)C)c2)N(C(=O)C(=O)Nc2cnc(N)c3c2cnn3C)C1] | 197 | (abs) | b | aa | aaa | C | A* |
| 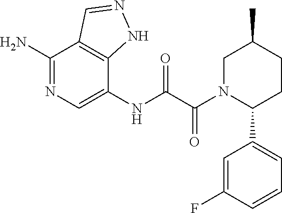 [SMILES: C[C@H]1CC[C@H](c2cccc(F)c2)N(C(=O)C(=O)Nc2cnc(N)c3cn[nH]c23)C1] | 198 | (abs) | b | aa | aaa | B | A* |
| 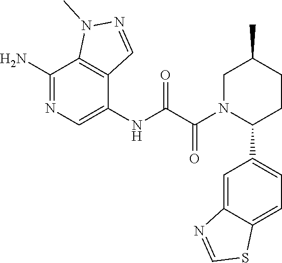 [SMILES: C[C@H]1CC[C@H](c2ccc3scnc3c2)N(C(=O)C(=O)Nc2cnc(N)c3c2cnn3C)C1] | 199 | (abs) | b | aa | aaa | C | A* |
| 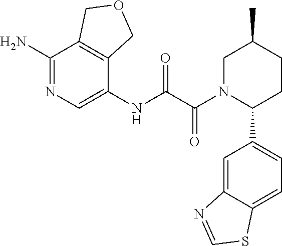 [SMILES: C[C@H]1CC[C@H](c2ccc3scnc3c2)N(C(=O)C(=O)Nc2cnc(N)c3c2COC3)C1] | 200 | (abs) | c | aa | aaa | B | A* |
| 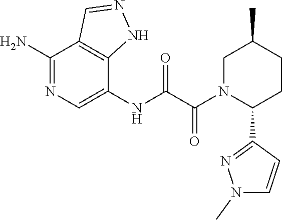 [SMILES: C[C@H]1CC[C@H](c2ccn(C)n2)N(C(=O)C(=O)Nc2cnc(N)c3cn[nH]c23)C1] | 201 | (abs) | b | aa | aaa | C | A* |
| 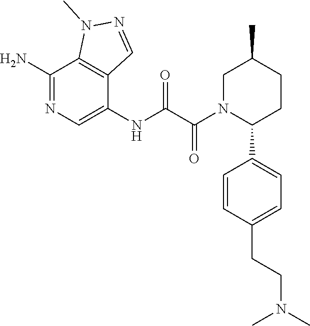 [SMILES: C[C@H]1CC[C@H](c2ccc(CCN(C)C)cc2)N(C(=O)C(=O)Nc2cnc(N)c3c2cnn3C)C1] | 202 | (abs) | b | aa | aaa | C | A* |
| 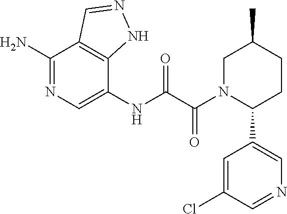 [SMILES: C[C@H]1CC[C@H](c2cncc(Cl)c2)N(C(=O)C(=O)Nc2cnc(N)c3cn[nH]c23)C1] | 203 | (abs) | b | aa | aaa | A | A* |
| 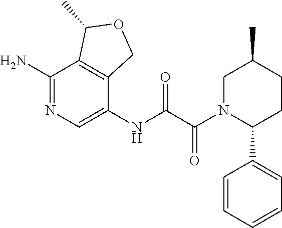 [SMILES: C[C@H]1CC[C@H](c2ccccc2)N(C(=O)C(=O)Nc2cnc(N)c3c2CO[C@H]3C)C1] | 204 | (abs) on piperidine, (or) on dihydrofuran | c | cc | ccc | C | C* |
| 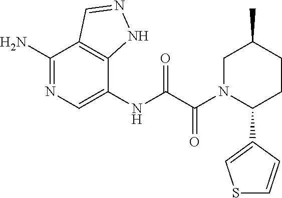 [SMILES: C[C@H]1CC[C@H](c2ccsc2)N(C(=O)C(=O)Nc2cnc(N)c3cn[nH]c23)C1] | 205 | (abs) | b | aa | aaa | A | A* |
| 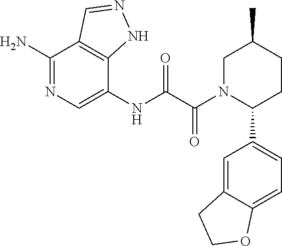 [SMILES: C[C@H]1CC[C@H](c2ccc3c(c2)CCO3)N(C(=O)C(=O)Nc2cnc(N)c3cn[nH]c23)C1] | 206 | (abs) | b | aa | aaa | B | A* |
| 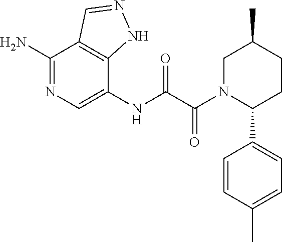 [SMILES: Cc1ccc([C@H]2CC[C@H](C)CN2C(=O)C(=O)Nc2cnc(N)c3cn[nH]c23)cc1] | 207 | (abs) | b | aa | aaa | B | A* |
| 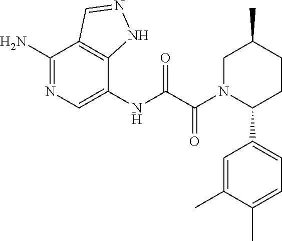 [SMILES: Cc1ccc([C@H]2CC[C@H](C)CN2C(=O)C(=O)Nc2cnc(N)c3cn[nH]c23)cc1C] | 208 | (abs) | b | aa | aaa | B | A* |
| 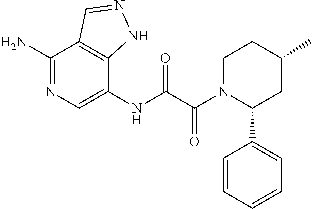 [SMILES: C[C@H]1CCN(C(=O)C(=O)Nc2cnc(N)c3cn[nH]c23)[C@@H](c2ccccc2)C1] | 209 | (and) | c | aa | aaa | A | A* |
| 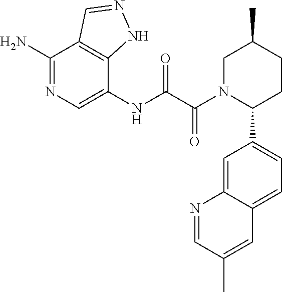 [SMILES: Cc1cnc2cc([C@H]3CC[C@H](C)CN3C(=O)C(=O)Nc3cnc(N)c4cn[nH]c34)ccc2c1] | 210 | (abs) | a | aa | aaa | C | A* |
| 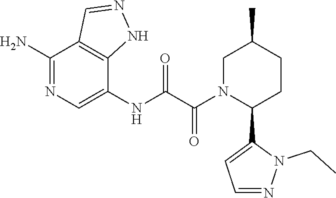 [SMILES: CCn1nccc1[C@@H]1CC[C@H](C)CN1C(=O)C(=O)Nc1cnc(N)c2cn[nH]c12] | 211 | (or) | c | aa | aaa | A | A* |
| 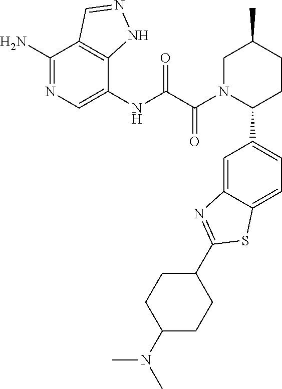 [SMILES: C[C@H]1CC[C@H](c2ccc3sc(C4CCC(N(C)C)CC4)nc3c2)N(C(=O)C(=O)Nc2cnc(N)c3cn[nH]c23)C1] | 212 | (abs) on piperidine, (and) on cyclohexyl | b | aa | aaa | C | A* |
| 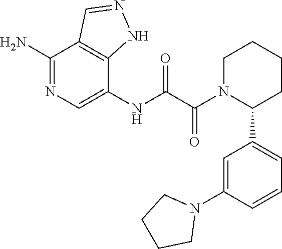 [SMILES: Nc1ncc(NC(=O)C(=O)N2CCCC[C@@H]2c2cccc(N3CCCC3)c2)c2[nH]ncc12] | 213 | (or) | c | bb | ccc | A | B* |
| 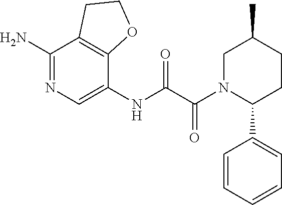 [SMILES: C[C@H]1CC[C@H](c2ccccc2)N(C(=O)C(=O)Nc2cnc(N)c3c2OCC3)C1] | 214 | (abs) | c | bb | ccc | B | B* |
| 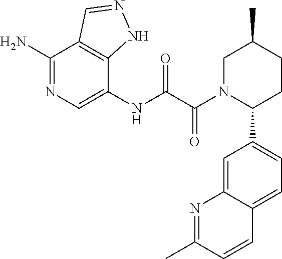 [SMILES: Cc1ccc2ccc([C@H]3CC[C@H](C)CN3C(=O)C(=O)Nc3cnc(N)c4cn[nH]c34)cc2n1] | 215 | (abs) | b | aa | aaa | C | A* |
| 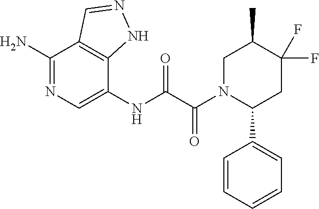 [SMILES: C[C@@H]1CN(C(=O)C(=O)Nc2cnc(N)c3cn[nH]c23)[C@@H](c2ccccc2)CC1(F)F] | 216 | (and) | b | aa | aaa | C | A* |
| 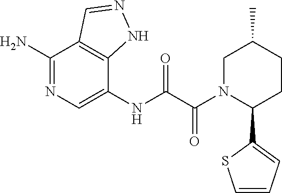 [SMILES: C[C@@H]1CC[C@@H](c2cccs2)N(C(=O)C(=O)Nc2cnc(N)c3cn[nH]c23)C1] | 217 | (and) | b | aa | aaa | B | A* |
| 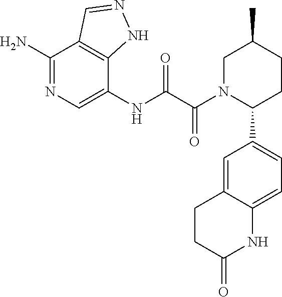 [SMILES: C[C@H]1CC[C@H](c2ccc3c(c2)CCC(=O)N3)N(C(=O)C(=O)Nc2cnc(N)c3cn[nH]c23)C1] | 218 | (abs) | c | bb | bbb | C | A* |
| 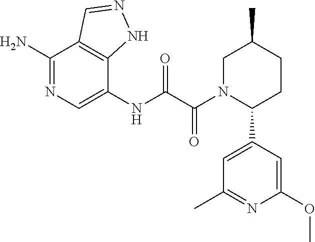 [SMILES: COc1cc([C@H]2CC[C@H](C)CN2C(=O)C(=O)Nc2cnc(N)c3cn[nH]c23)cc(C)n1] | 219 | (abs) | c | aa | aaa | A | A* |
| 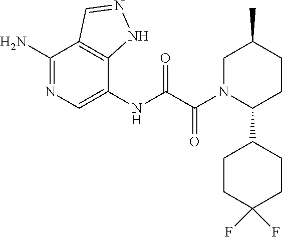 [SMILES: C[C@H]1CC[C@H](C2CCC(F)(F)CC2)N(C(=O)C(=O)Nc2cnc(N)c3cn[nH]c23)C1] | 220 | (abs) | c | bb | bbb | B | A* |
| 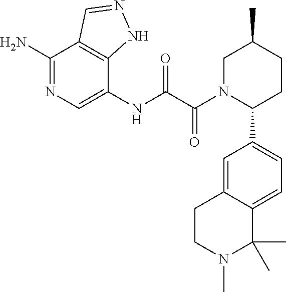 [SMILES: C[C@H]1CC[C@H](c2ccc3c(c2)CCN(C)C3(C)C)N(C(=O)C(=O)Nc2cnc(N)c3cn[nH]c23)C1] | 221 | (abs) | c | bb | aaa | B | A* |
| 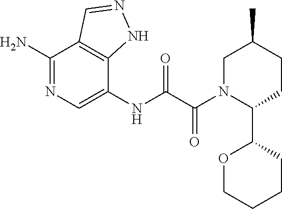 [SMILES: C[C@H]1CC[C@H]([C@@H]2CCCCO2)N(C(=O)C(=O)Nc2cnc(N)c3cn[nH]c23)C1] | 222 | (abs) on piperidine (and) on tetrahydropyran | c | aa | aaa | A | A* |
| 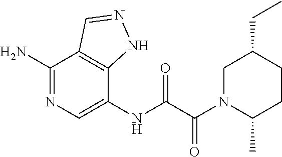 [SMILES: CC[C@@H]1CC[C@H](C)N(C(=O)C(=O)Nc2cnc(N)c3cn[nH]c23)C1] | 223 | (and) | c | aa | bbb | A | B* |
| 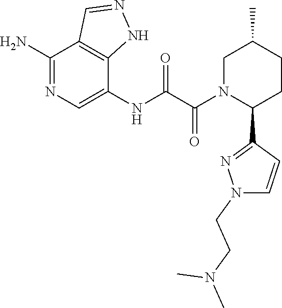 [SMILES: C[C@@H]1CC[C@@H](c2ccn(CCN(C)C)n2)N(C(=O)C(=O)Nc2cnc(N)c3cn[nH]c23)C1] | 224 | (and) | c | bb | ccc | C | A* |
| 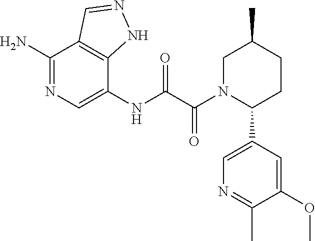 [SMILES: COc1cc([C@H]2CC[C@H](C)CN2C(=O)C(=O)Nc2cnc(N)c3cn[nH]c23)cnc1C] | 225 | (abs) | c | aa | aaa | B | A* |
| 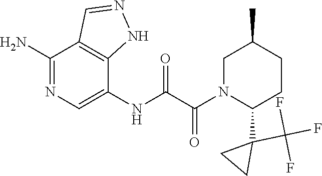 [SMILES: C[C@H]1CC[C@H](C2(C(F)(F)F)CC2)N(C(=O)C(=O)Nc2cnc(N)c3cn[nH]c23)C1] | 226 | (abs) | c | bb | ccc | B | B* |
| 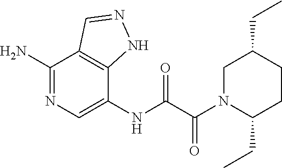 [SMILES: CC[C@@H]1CC[C@H](CC)N(C(=O)C(=O)Nc2cnc(N)c3cn[nH]c23)C1] | 227 | (and) | b | aa | aaa | B | A* |
| 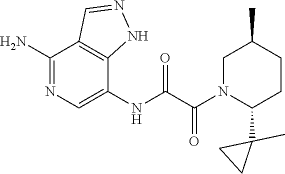 [SMILES: C[C@H]1CC[C@H](C2(C)CC2)N(C(=O)C(=O)Nc2cnc(N)c3cn[nH]c23)C1] | 228 | (abs) | c | aa | aaa | A | A* |
| 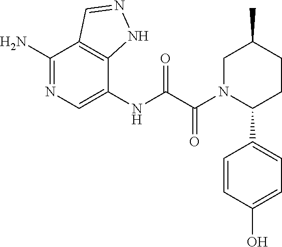 [SMILES: C[C@H]1CC[C@H](c2ccc(O)cc2)N(C(=O)C(=O)Nc2cnc(N)c3cn[nH]c23)C1] | 229 | (abs) | | | | | A* |
| 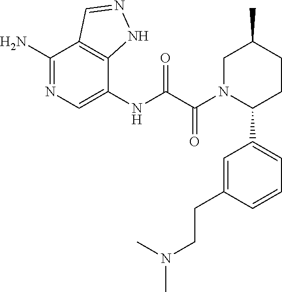 [SMILES: C[C@H]1CC[C@H](c2cccc(CCN(C)C)c2)N(C(=O)C(=O)Nc2cnc(N)c3cn[nH]c23)C1] | 230 | (abs) | c | bb | aaa | C | A* |
| 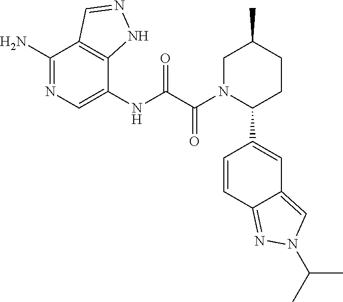 [SMILES: CC(C)n1cc2cc([C@H]3CC[C@H](C)CN3C(=O)C(=O)Nc3cnc(N)c4cn[nH]c34)ccc2n1] | 231 | (abs) | b | aa | aaa | A | A* |
| 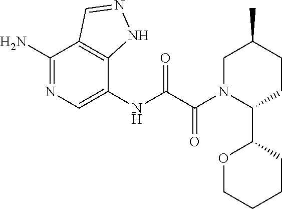 [SMILES: C[C@H]1CC[C@H]([C@@H]2CCCCO2)N(C(=O)C(=O)Nc2cnc(N)c3cn[nH]c23)C1] | 232 | (abs) on piperidine (or) on tetrahydropyran | c | aa | aaa | A | A* |
| 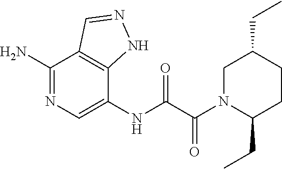 [SMILES: CC[C@@H]1CC[C@@H](CC)N(C(=O)C(=O)Nc2cnc(N)c3cn[nH]c23)C1] | 233 | (and) | c | aa | aaa | B | A* |
| 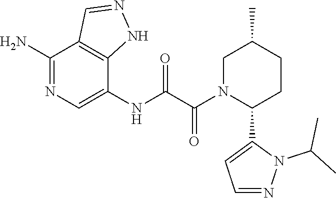 [SMILES: CC(C)n1nccc1[C@H]1CC[C@@H](C)CN1C(=O)C(=O)Nc1cnc(N)c2cn[nH]c12] | 234 | (or) | c | cc | ccc | B | B* |
| 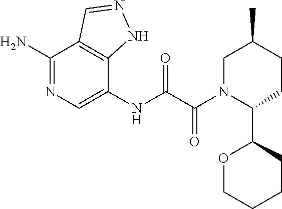 [SMILES: C[C@H]1CC[C@H]([C@H]2CCCCO2)N(C(=O)C(=O)Nc2cnc(N)c3cn[nH]c23)C1] | 235 | (abs) on piperidine (or) on tetrahydropyran | c | aa | aaa | A | A* |
| 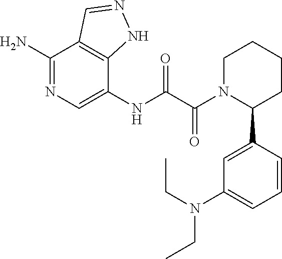 [SMILES: CCN(CC)c1cccc([C@@H]2CCCCN2C(=O)C(=O)Nc2cnc(N)c3cn[nH]c23)c1] | 236 | (or) | c | aa | aaa | A | A* |
| 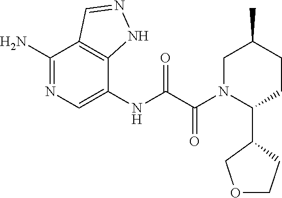 [SMILES: C[C@H]1CC[C@H]([C@@H]2CCOC2)N(C(=O)C(=O)Nc2cnc(N)c3cn[nH]c23)C1] | 237 | (abs) on piperidine (or) on tetrahydrofuran | c | aa | ccc | A | B* |
| 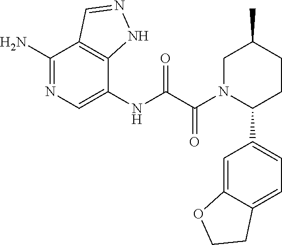 [SMILES: C[C@H]1CC[C@H](c2ccc3c(c2)OCC3)N(C(=O)C(=O)Nc2cnc(N)c3cn[nH]c23)C1] | 238 | (abs) | b | aa | aaa | A | A* |
| 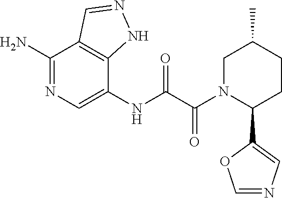 [SMILES: C[C@@H]1CC[C@@H](c2cnco2)N(C(=O)C(=O)Nc2cnc(N)c3cn[nH]c23)C1] | 239 | (and) | c | bb | aaa | C | A* |
| 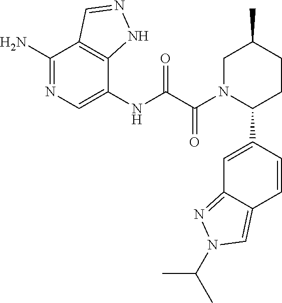 [SMILES: CC(C)n1cc2ccc([C@H]3CC[C@H](C)CN3C(=O)C(=O)Nc3cnc(N)c4cn[nH]c34)cc2n1] | 240 | (abs) | a | aa | aaa | C | A* |
| 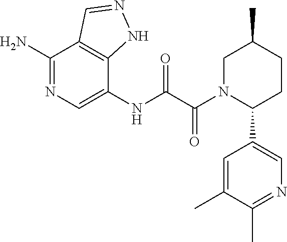 [SMILES: Cc1cc([C@H]2CC[C@H](C)CN2C(=O)C(=O)Nc2cnc(N)c3cn[nH]c23)cnc1C] | 241 | (abs) | c | aa | aaa | A | A* |
| 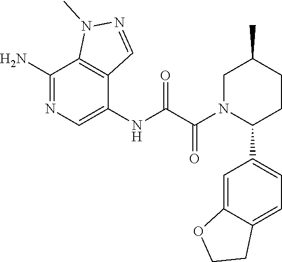 [SMILES: C[C@H]1CC[C@H](c2ccc3c(c2)OCC3)N(C(=O)C(=O)Nc2cnc(N)c3c2cnn3C)C1] | 242 | (abs) | | | | | A* |
| 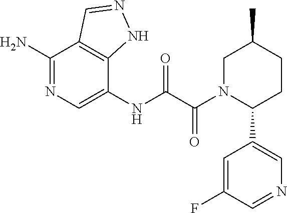 [SMILES: C[C@H]1CC[C@H](c2cncc(F)c2)N(C(=O)C(=O)Nc2cnc(N)c3cn[nH]c23)C1] | 243 | (abs) | c | aa | aaa | A | A* |
| 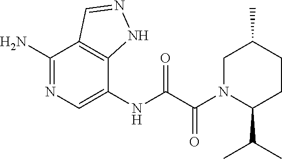 [SMILES: CC(C)[C@@H]1CC[C@@H](C)CN1C(=O)C(=O)Nc1cnc(N)c2cn[nH]c12] | 244 | (and) | c | aa | aaa | A | A* |
| 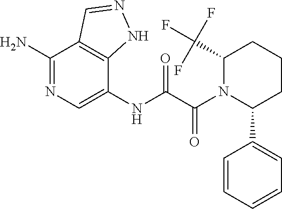 [SMILES: Nc1ncc(NC(=O)C(=O)N2[C@@H](c3ccccc3)CCC[C@H]2C(F)(F)F)c2[nH]ncc12] | 245 | (rac) | c | aa | aaa | A | A* |
| 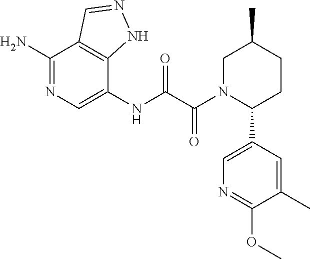 [SMILES: COc1ncc([C@H]2CC[C@H](C)CN2C(=O)C(=O)Nc2cnc(N)c3cn[nH]c23)cc1C] | 246 | (abs) | b | aa | aaa | B | A* |
| 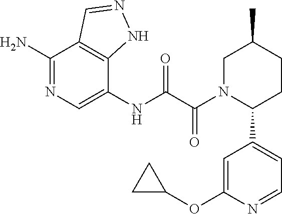 [SMILES: C[C@H]1CC[C@H](c2ccnc(OC3CC3)c2)N(C(=O)C(=O)Nc2cnc(N)c3cn[nH]c23)C1] | 247 | (abs) | b | aa | aaa | B | A* |
| 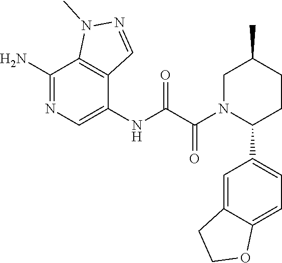 [SMILES: C[C@H]1CC[C@H](c2ccc3c(c2)CCO3)N(C(=O)C(=O)Nc2cnc(N)c3c2cnn3C)C1] | 248 | (abs) | | | | | A* |
| 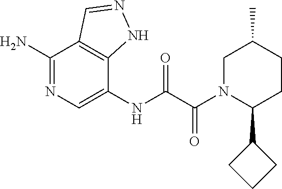 [SMILES: C[C@@H]1CC[C@@H](C2CCC2)N(C(=O)C(=O)Nc2cnc(N)c3cn[nH]c23)C1] | 249 | (and) | c | aa | aaa | A | A* |
| 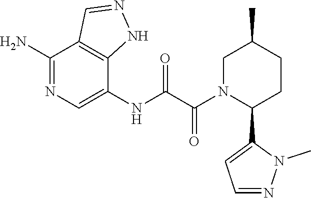 [SMILES: C[C@H]1CC[C@@H](c2ccnn2C)N(C(=O)C(=O)Nc2cnc(N)c3cn[nH]c23)C1] | 250 | (or) | b | aa | aaa | B | A* |
| 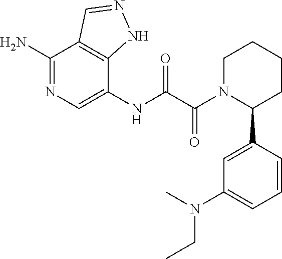 [SMILES: CCN(C)c1cccc([C@@H]2CCCCN2C(=O)C(=O)Nc2cnc(N)c3cn[nH]c23)c1] | 251 | (or) | b | aa | aaa | B | A* |
| 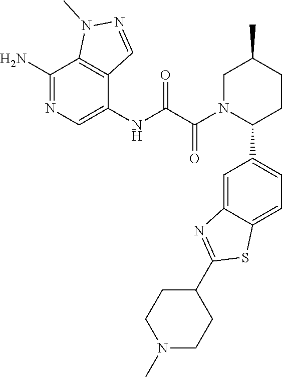 [SMILES: C[C@H]1CC[C@H](c2ccc3sc(C4CCN(C)CC4)nc3c2)N(C(=O)C(=O)Nc2cnc(N)c3c2cnn3C)C1] | 252 | (abs) | | | | | A* |
| 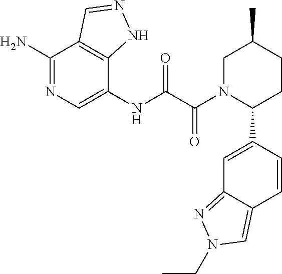 [SMILES: CCn1cc2ccc([C@H]3CC[C@H](C)CN3C(=O)C(=O)Nc3cnc(N)c4cn[nH]c34)cc2n1] | 253 | (abs) | a | aa | aaa | C | A* |
| 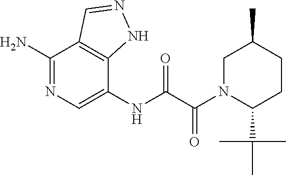 [SMILES: C[C@H]1CC[C@H](C(C)(C)C)N(C(=O)C(=O)Nc2cnc(N)c3cn[nH]c23)C1] | 254 | (abs) | c | bb | ccc | B | B* |
| 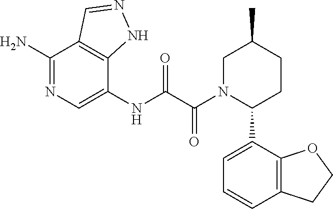 [SMILES: C[C@H]1CC[C@H](c2cccc3c2OCC3)N(C(=O)C(=O)Nc2cnc(N)c3cn[nH]c23)C1] | 255 | (abs) | b | aa | aaa | B | A* |
| 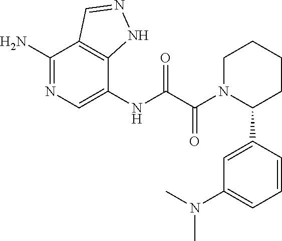 [SMILES: CN(C)c1cccc([C@H]2CCCCN2C(=O)C(=O)Nc2cnc(N)c3cn[nH]c23)c1] | 256 | (or) | c | bb | ccc | A | B* |
| 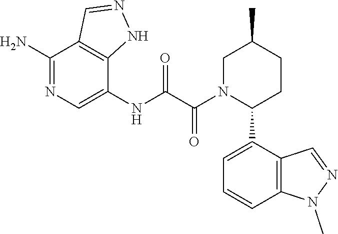 [SMILES: C[C@H]1CC[C@H](c2cccc3c2cnn3C)N(C(=O)C(=O)Nc2cnc(N)c3cn[nH]c23)C1] | 257 | (abs) | b | aa | aaa | C | A* |
| 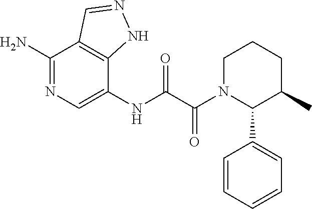 [SMILES: C[C@@H]1CCCN(C(=O)C(=O)Nc2cnc(N)c3cn[nH]c23)[C@H]1c1ccccc1] | 258 | (and) | c | bb | bbb | A | B* |
| 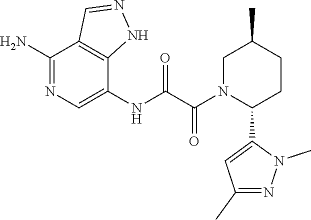 [SMILES: Cc1cc([C@H]2CC[C@H](C)CN2C(=O)C(=O)Nc2cnc(N)c3cn[nH]c23)n(C)n1] | 259 | (abs) | c | aa | bbb | A | B* |
| 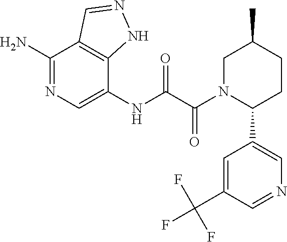 [SMILES: C[C@H]1CC[C@H](c2cncc(C(F)(F)F)c2)N(C(=O)C(=O)Nc2cnc(N)c3cn[nH]c23)C1] | 260 | (abs) | b | aa | aaa | B | A* |
| 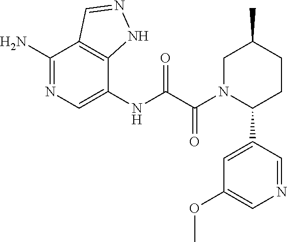 [SMILES: COc1cncc([C@H]2CC[C@H](C)CN2C(=O)C(=O)Nc2cnc(N)c3cn[nH]c23)c1] | 261 | (abs) | c | aa | aaa | A | A* |
| 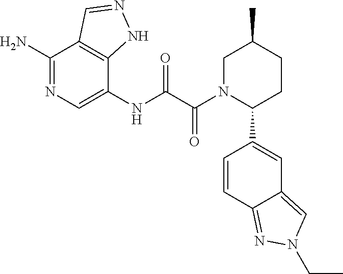 [SMILES: CCn1cc2cc([C@H]3CC[C@H](C)CN3C(=O)C(=O)Nc3cnc(N)c4cn[nH]c34)ccc2n1] | 262 | (abs) | b | aa | aaa | C | A* |
| 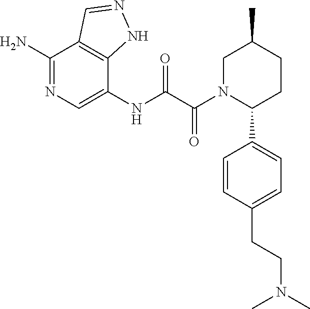 [SMILES: C[C@H]1CC[C@H](c2ccc(CCN(C)C)cc2)N(C(=O)C(=O)Nc2cnc(N)c3cn[nH]c23)C1] | 263 | (abs) | b | aa | aaa | C | A* |
| 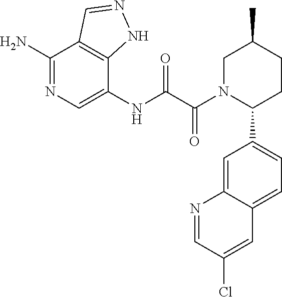 [SMILES: C[C@H]1CC[C@H](c2ccc3cc(Cl)cnc3c2)N(C(=O)C(=O)Nc2cnc(N)c3cn[nH]c23)C1] | 264 | (abs) | b | aa | aaa | B | A* |
| 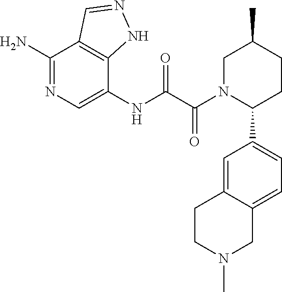 [SMILES: C[C@H]1CC[C@H](c2ccc3c(c2)CCN(C)C3)N(C(=O)C(=O)Nc2cnc(N)c3cn[nH]c23)C1] | 265 | (abs) | b | aa | aaa | C | A* |
| 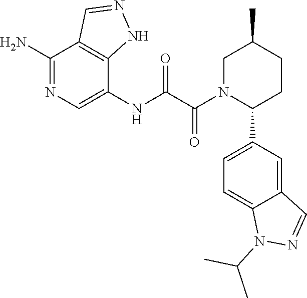 [SMILES: CC(C)n1ncc2cc([C@H]3CC[C@H](C)CN3C(=O)C(=O)Nc3cnc(N)c4cn[nH]c34)ccc21] | 266 | (abs) | b | aa | aaa | C | A* |
| 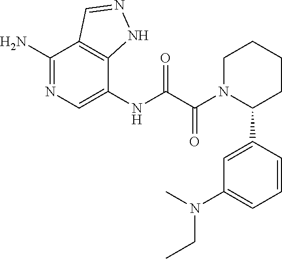 [SMILES: CCN(C)c1cccc([C@H]2CCCCN2C(=O)C(=O)Nc2cnc(N)c3cn[nH]c23)c1] | 267 | (or) | c | aa | aaa | A | A* |
| 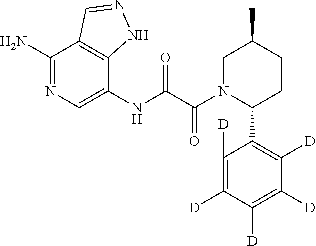 [SMILES: [2H]c1c([2H])c([2H])c([C@H]2CC[C@H](C)CN2C(=O)C(=O)Nc2cnc(N)c3cn[nH]c23)c([2H])c1[2H]] | 268 | (abs) | b | aa | aaa | B | A* |
| 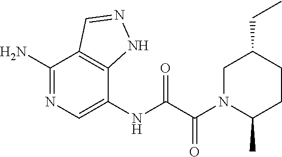 [SMILES: CC[C@@H]1CC[C@@H](C)N(C(=O)C(=O)Nc2cnc(N)c3cn[nH]c23)C1] | 269 | (and) | c | bb | ccc | A | B* |
| 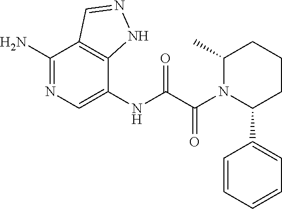 [SMILES: C[C@@H]1CCC[C@H](c2ccccc2)N1C(=O)C(=O)Nc1cnc(N)c2cn[nH]c12] | 270 | (and) | c | aa | aaa | A | A* |
| 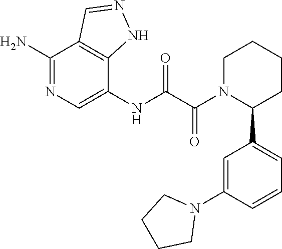 [SMILES: Nc1ncc(NC(=O)C(=O)N2CCCC[C@H]2c2cccc(N3CCCC3)c2)c2[nH]ncc12] | 271 | (or) | c | aa | aaa | A | A* |
| 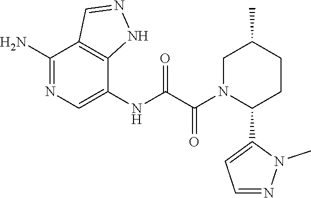 [SMILES: C[C@@H]1CC[C@H](c2ccnn2C)N(C(=O)C(=O)Nc2cnc(N)c3cn[nH]c23)C1] | 272 | (or) | c | bb | ccc | B | B* |
| 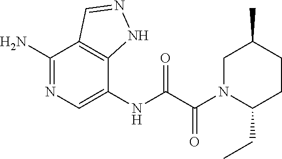 [SMILES: CC[C@H]1CC[C@H](C)CN1C(=O)C(=O)Nc1cnc(N)c2cn[nH]c12] | 273 | (abs) | c | aa | aaa | A | A* |
| 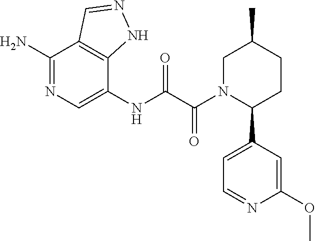 [SMILES: COc1cc([C@@H]2CC[C@H](C)CN2C(=O)C(=O)Nc2cnc(N)c3cn[nH]c23)ccn1] | 274 | (or) | c | bb | ccc | B | B* |
| 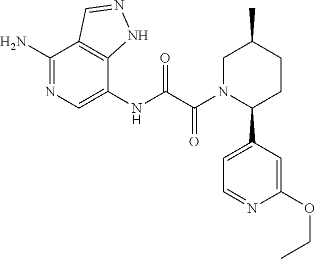 [SMILES: CCOc1cc([C@@H]2CC[C@H](C)CN2C(=O)C(=O)Nc2cnc(N)c3cn[nH]c23)ccn1] | 275 | (or) | c | bb | ccc | C | B* |
| 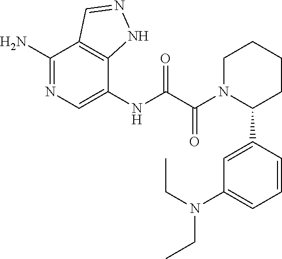 [SMILES: CCN(CC)c1cccc([C@H]2CCCCN2C(=O)C(=O)Nc2cnc(N)c3cn[nH]c23)c1] | 276 | (or) | c | aa | aaa | A | A* |
| 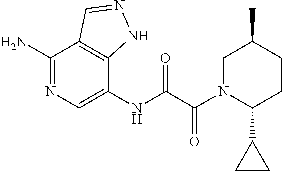 [SMILES: C[C@H]1CC[C@H](C2CC2)N(C(=O)C(=O)Nc2cnc(N)c3cn[nH]c23)C1] | 277 | (abs) | c | aa | aaa | A | A* |
| 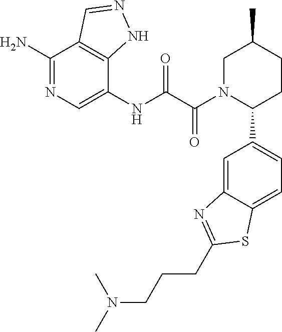 [SMILES: C[C@H]1CC[C@H](c2ccc3sc(CCCN(C)C)nc3c2)N(C(=O)C(=O)Nc2cnc(N)c3cn[nH]c23)C1] | 278 | (abs) | b | aa | aaa | C | A* |
| 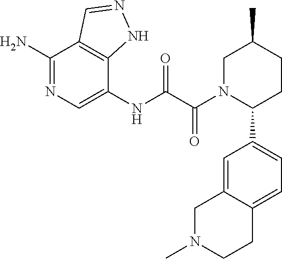 [SMILES: C[C@H]1CC[C@H](c2ccc3c(c2)CN(C)CC3)N(C(=O)C(=O)Nc2cnc(N)c3cn[nH]c23)C1] | 279 | (abs) | b | aa | aaa | C | A* |
| 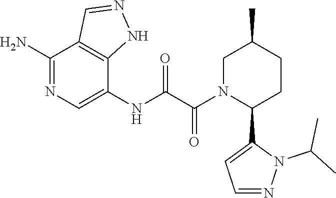 [SMILES: CC(C)n1nccc1[C@@H]1CC[C@H](C)CN1C(=O)C(=O)Nc1cnc(N)c2cn[nH]c12] | 280 | (or) | c | bb | bbb | A | A* |
| 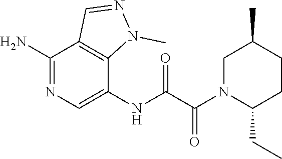 [SMILES: CC[C@H]1CC[C@H](C)CN1C(=O)C(=O)Nc1cnc(N)c2cnn(C)c12] | 281 | (abs) | c | cc | ccc | C | C* |
| 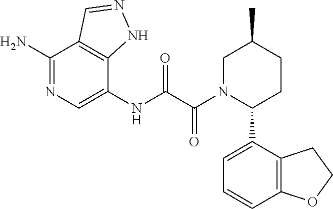 [SMILES: C[C@H]1CC[C@H](c2cccc3c2CCO3)N(C(=O)C(=O)Nc2cnc(N)c3cn[nH]c23)C1] | 282 | (abs) | b | aa | aaa | A | A* |
| 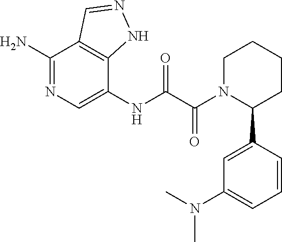 [SMILES: CN(C)c1cccc([C@@H]2CCCCN2C(=O)C(=O)Nc2cnc(N)c3cn[nH]c23)c1] | 283 | (or) | b | aa | aaa | A | A* |
| 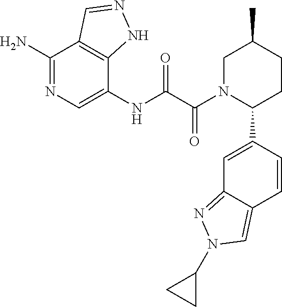 [SMILES: C[C@H]1CC[C@H](c2ccc3cn(C4CC4)nc3c2)N(C(=O)C(=O)Nc2cnc(N)c3cn[nH]c23)C1] | 284 | (abs) | a | aa | aaa | C | A* |
| 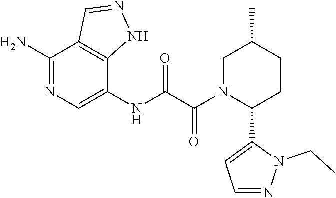 [SMILES: CCn1nccc1[C@H]1CC[C@@H](C)CN1C(=O)C(=O)Nc1cnc(N)c2cn[nH]c12] | 285 | (or) | c | bb | ccc | A | B* |
| 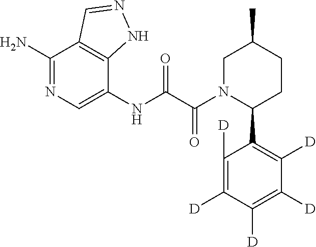 [SMILES: [2H]c1c([2H])c([2H])c([C@@H]2CC[C@H](C)CN2C(=O)C(=O)Nc2cnc(N)c3cn[nH]c23)c([2H])c1[2H]] | 286 | (abs) | c | bb | ccc | B | B* |
| 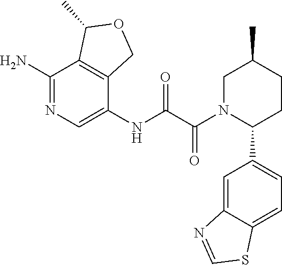 [SMILES: C[C@H]1CC[C@H](c2ccc3scnc3c2)N(C(=O)C(=O)Nc2cnc(N)c3c2CO[C@H]3C)C1] | 287 | (abs) on piperidine (or) on dihydrofuran) | c | aa | aaa | B | A* |
| 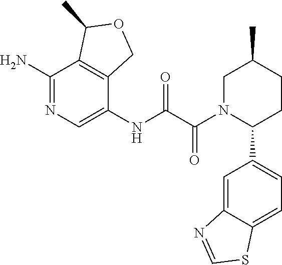 [SMILES: C[C@H]1CC[C@H](c2ccc3scnc3c2)N(C(=O)C(=O)Nc2cnc(N)c3c2CO[C@@H]3C)C1] | 288 | (abs) on piperidine (or) on dihydrofuran) | c | cc | ccc | B | C* |

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In another embodiment, provided is a pharmaceutical composition comprising a pharmaceutically acceptable carrier and an effective amount of a compound described herein (e.g., a compound of Formula (A) or Formula (I) to (III7a) or a compound of Table 1), or a pharmaceutically acceptable salt thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as α-, β-, and γ-cyclodextrin, or chemically modified derivatives such as hydroxyalkylcyclodextrins, including 2 and 3 hydroxypropyl-β-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In one embodiment, with respect to the pharmaceutical composition, the carrier is a parenteral carrier, oral or topical carrier.

Also provided is a compound described herein (e.g., a compound of Formula (A) or Formula (I) to (III7a) or a compound of Table 1, or pharmaceutically acceptable salts thereof) (or pharmaceutical composition thereof) for use as a pharmaceutical or a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Formula (A) or

Formula (I) to (III7a) or a compound of Table 1, or pharmaceutically acceptable salts thereof) (or pharmaceutical composition thereof) for use in the treatment of an MTAP-deficient and/or an MTA-accumulating disease in a subject in need thereof. In one embodiment, the disease is a proliferating disease. In a further embodiment, the disease is an MTAP-deficient and/or MTA-accumulating cancer. In one embodiment, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Formula (A) or Formula (I) to (III7a) or a compound of Table 1, or pharmaceutically acceptable salts thereof) (or pharmaceutical composition thereof) for use in the manufacturing of a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Generally, the compounds provided herein are administered in an effective amount (e.g., a therapeutically effective amount). The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable non irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds described herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided is the pharmaceutically acceptable acid addition salt of a compound described herein (e.g., compound of Formula (A) or Formula (I) to (III7a) or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment and Use

Treatment of MTAP-Deficient and/or MTA-Accumulating Proliferation Disorders

5-Methylthioadenosine phosphorylase (MTAP) catalyzes the reversible phosphorylation of S-methyl-5′-thioadenosine (MTA) to adenine and 5-methylthioribose-1-phosphate. MTAP-deletion is a common genetic event in human cancer. MTAP deletion frequency in a subset of human cancers is described in Cerami et al., Cancer Discov. (2012); 2 (5): 401-4; Gao et al., Sci Signal. (2013); 6 (269): pl1; and Lee et al., Nat. Gen. (2014) 46 (11): 1227-32. For example, more than 50% of malignant peripheral nerve sheath tumor (MPNST) have deletions in MTAP (Lee et al., Nat. Gen. (2014)). Other cancers with high MTAP deletion frequencies are glioblastoma (GBM), mesothelioma, bladder cancer, pancreatic cancer, esophageal cancer, squamous lung cancer, melanoma, diffuse large B cell lymphoma (DLBCL), head and neck cancer, cholangiocarcinoma, lung adenoma, sarcoma, stomach cancer, glioma, adrenal carcinoma, thymoma, breast cancer, liver cancer, ovarian cancer, renal papillary cancer, uterine cancer, prostate cancer, and renal clear cell cancer. MTAP deletion in cells is one of the mechanisms that leads to MTAP-deficiency, increased intracellular MTA accumulation, and confers enhanced dependency on the protein arginine methyltransferase 5 (PRMT5) in cancer cells. Other mechanisms leading to MTAP deficiency include, inter alia, MTAP translocations and MTAP epigenetic silencing which could also lead to MTAP-null and/or MTAP deficient tumors. PRMT5 mediates the formation of symmetric dimethylarginine (SDMA); thus, the PRMT5 activity can be assessed by measuring the SDMA levels using the antibody against an SDMA or SDMA modified polypeptide.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a compound of the present disclosure (e.g., a compound of Formula (A) or Formula (I) to (III7a) or a compound of Table 1) or a pharmaceutically acceptable salt thereof.

In some embodiments, provided is a compound of the present disclosure (e.g., a compound of Formula (I) and Formula (A) or a compound of Table 1), or a pharmaceutical composition comprising a compound of Formula (I) and Formula (A) of the present disclosure for use in a method of treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a compound of the present disclosure (e.g., a compound of Formula (I) and Formula (A) or a compound of Table 1), or a pharmaceutical composition comprising a compound of Formula (I) and Formula (A) of the present disclosure for use in the manufacturing of a medicament for treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a use of a compound of the present disclosure (e.g., a compound of Formula (I) and Formula (A) or a compound of Table 1), or of a pharmaceutical composition comprising a compound of Formula (I) and Formula (A) of the present disclosure in a method of treating human or animal subjects having or having been diagnosed with an MTAP-deficiency-related and/or MTA-accumulating proliferative disorder (e.g., cancer). In some embodiments, the use is of a therapeutically effective amount of the compound or composition.

In some embodiments, provided is use of a compound of the present disclosure (e.g., a compound of Formula (I) and Formula (A) or a compound of Table 1), or of a pharmaceutical composition comprising a compound of Formula (I) and Formula (A) of the present disclosure in the manufacturing of a medicament for treating human or animal subjects having or having been diagnosed with an MTAP-deficiency-related and/or MTA-accumulating proliferative disorder (e.g., cancer). In some embodiments, the use is of a therapeutically effective amount of the compound or composition.

In one embodiment, provided are methods for treating an MTAP-deficiency-related and/or MTA-accumulating proliferative disorder (e.g., cancer) in a subject in need thereof comprising administering to the subject an effective amount (e.g., a therapeutically effective amount) of a compound of the present disclosure (e.g., compound of Formula (A) or Formula (I) to (III7a) or a compound of Table 1) or a pharmaceutically acceptable salt thereof.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of pharmaceutical composition of the present disclosure (e.g., a composition comprising a compound of Formula (A) or Formula (I) to (III7a) or a compound of Table 1, or pharmaceutically acceptable salts thereof and a pharmaceutically acceptable carrier). In one embodiment, the compound or composition is administered in combination with a second therapeutic agent.

In one embodiment, provided are methods of treating an MTAP-deficiency-related and/or MTA-accumulating proliferative disorder (e.g., cancer) in a subject in need thereof comprising administering to the subject a therapeutically effective amount of pharmaceutical composition of the present disclosure (e.g., a composition comprising a compound of Formula (A) or Formula (I) to (III7a) or a compound of Table 1, or pharmaceutically acceptable salts thereof and a pharmaceutically acceptable carrier). In one embodiment, the compound or composition is administered in combination with a second therapeutic agent.

In some embodiments, the subject is human.

In certain embodiments, the disease is an MTAP-deficient and/or MTA-accumulating cancer.

In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, the cancer is an MTAP-deficient and/or MTA-accumulating glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Formula (A) or Formula (I) to (III7a) or a compound of Table 1, or pharmaceutically acceptable salts thereof) described herein can be used in a method of inhibiting proliferation of MTAP-deficient cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Formula (A) or Formula (I) to (III7a) or a compound of Table 1, or pharmaceutically acceptable salts thereof) in an amount that is effective to inhibit proliferation of the MTAP-deficient cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Formula (A) or Formula (I) to (III7a) or a compound of Table 1, or pharmaceutically acceptable salts thereof) described herein can be used in a method of inhibiting proliferation of MTA-accumulating cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Formula (A) or Formula (I) to (III7a) or a compound of Table 1, or pharmaceutically acceptable salts thereof) in an amount that is effective to inhibit proliferation of the MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) described herein can be used in a method of inhibiting proliferation of MTAP deficient and/or MTA-accumulating cells in a subject in need thereof, the method comprising the step of administering to the subject, a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) in an amount that is effective to inhibit proliferation of the MTAP deficient and/or MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Combination Therapies

In some embodiments, provided are methods of treatment of MTAP-deficient and/or MTA accumulating proliferative disorders (e.g., cancers) with a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) in combination with one or more therapeutic agent.

In some embodiments, provided are methods of treatment of MTAP-deficient and/or MTA accumulating proliferative disorders (e.g., cancers) with a PRMT5 inhibitor described herein (e.g., a compound of Formula (A) or Formula (I) to (III7a) or a compound of Table 1, or pharmaceutically acceptable salts thereof) in combination with a second therapeutic agent. In some embodiments, provided are methods of treatment of MTAP-deficient and/or MTA accumulating proliferative disorders (e.g., cancers) with PRMT5 inhibitor described herein (e.g., a compound of Formula (A) or Formula (I) to (III7a) or a compound of Table 1, or pharmaceutically acceptable salts thereof) in combination with a second therapeutic agent and a third therapeutic agent. In some embodiments, provided are methods of treatment of MTAP-deficient and/or MTA accumulating proliferative disorders (e.g., cancers) with PRMT5 inhibitor described herein (e.g., a compound of Formula (A) or Formula (I) to (III7a) or a compound of Table 1, or pharmaceutically acceptable salts thereof) in combination with a second therapeutic agent, a third therapeutic agent, and a fourth therapeutic agent.

The term “Combination” refers to either a fixed combination in one dosage unit form, or a combined administration where a PRMT5 inhibitor described herein (e.g., a compound of Formula (A) or Formula (I) to (III7a) or a compound of Table 1, or pharmaceutically acceptable salts thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., PRMT5 inhibitors described herein (e.g., a compound of Formula (A) or Formula (I) to (III7a) or a compound of Table 1, or pharmaceutically acceptable salts thereof) and a combination partner, are both administered to a patient simultaneously in the form of a single entity or dosage. The term “non-fixed combination” means that the therapeutic agents, e.g., a PRMT5 inhibitor described herein (e.g., a compound of Formula (A) or Formula (I) to (III7a) or a compound of Table 1, or pharmaceutically acceptable salts thereof) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, PRMT5 inhibitors described herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a general chemotherapeutic agents selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-UR), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an EGFR-inhibitor (e.g., cetuximab, panitumimab, erlotinib, gefitinib and EGFRi NOS). In some embodiments, provided is a method of treating a disease or disorder (e.g., cancer) comprising administering or coadministering, in any order, to a patient in need thereof a PRMT5 inhibitor described herein (e.g., a compound of Formula (A) and Formula (I) to (III7a) or a compound of Table 1, or pharmaceutically acceptable salts thereof) and a MAPK-pathway inhibitor (e.g., BRAFi, panRAFi, MEKi, ERKi). In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a PI3K-mTOR pathway inhibitor (e.g., alpha-specific PI3Ki, pan-class I PI3Ki and mTOR/PI3Ki, particularly everolimus and analogues thereof).

MTAP-deletion can co-occur with mutations in the KRAS gene (e.g., KRASG12C). In some embodiments, provided is a method of treating a disease or disorder (e.g., cancer) comprising administering or coadministering, in any order, to a patient in need thereof a PRMT5 inhibitor described herein (e.g., a compound of Formula (I) and Formula (A) or a compound of Table 1, or pharmaceutically acceptable salts thereof), and a KRAS inhibitor (e.g., a pan-KRAS or a specific G12C, G12D, G13C inhibitor, e.g., adagrasib, sotorasib, LY3537982, RMC-6236, RMC-6291, RMC-9805, RMC-8839).

In some embodiments, provided is a method of treating a disease or disorder (e.g., cancer) comprising administering or coadministering, in any order, to a patient in need thereof a PRMT5 inhibitor described herein (e.g., a compound of Formula (I) and Formula (A) or a compound of Table 1, or pharmaceutically acceptable salts thereof), and a Spliceosome inhibitor (e.g., SF3b1 inhibitors; e.g., E7107).

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an HDAC inhibitor or DNA methyltransferase inhibitor. In some embodiments, the HDAC inhibitor is Trichostatin A. In some embodiments, the DNA methyltransferase inhibitor is 5-azacytidine.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a MAT2A inhibitor.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an inhibitor of a protein which interacts with or is required for PRMT5 function, including, but not limited to, pICIN, WDR77 or RIOK1.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an HDM2 inhibitor and/or 5-FU or other purine analogues (e.g., 6-thioguanine, 6-mercaptopurine).

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a CDK4 inhibitor, including, but not limited to, LEE011 or a CDK 4/6 inhibitor (e.g., palbociclib (Ibrance®), ribociclib (Kisqali®), and abemaciclib (Verzenio®)

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a targeted treatment contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and immunotherapy.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an immunotherapeutic agent.

In some embodiments, the immunotherapeutic agent is an anti-CTLA-4 antibody (e.g., ipilimumab, tremelimumab).

In some embodiments, the immunotherapeutic agent is an anti-PD-1 or anti-PD-L1 agent (e.g., an antibody). In some embodiments, the immunotherapeutic agent is an anti-PD-1 agent (e.g., an anti-PD-1 antibody, e.g., nivolumab (i.e., MDX-1106, BMS-936558, ONO-4538); CT-011; AMP-224; pembrolizumab (MK-3475); pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab). In some embodiments, the immunotherapeutic agent is an anti-PD-L1 agent (e.g., an anti-PD-L1 antibody, e.g., BMS936559 (i.e., MDX-1105); durvalumab (MEDI4736); avelumab (MSB0010718C); envafolimab; cosibelimab; sugemalimab, AUNP-12 or atezolizumab (MPDL-3280A) or an anti-PD-L1 small molecule (e.g., CA-170)).

In some embodiments, the immunotherapeutic agent is a checkpoint blocking antibody (e.g., anti-TIM3, anti-LAG3, anti-TIGIT including IMP321 and MGA271).

In some embodiments, the immunotherapeutic agent is a cell-based therapy. In some embodiments, the cell-based therapy is a CAR-T therapy.

In some embodiments, the immunotherapeutic agent is a co-stimulatory antibody (e.g., anti-4-1BB, anti-OX40, anti-GITR, anti-CD27, anti-CD40).

In some embodiments, the immunotherapeutic agent is a cancer vaccine such as a neoantigen. These vaccines can be developed using peptides or RNA (e.g., mRNA).

In some embodiments, the immunotherapeutic agent is an oncolytic virus.

In some embodiments, the immunotherapeutic agent is a STING pathway agonist. Exemplary STING agonists include MK-1454 and ADU-S100.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a disease-specific huMAB (e.g., an anti-HER3 huMAB).

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an ADC/ADCC contingent on the expression of relevant surface targets on target tumors of interest.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and one or more DNA damage pathway inhibitor. In some embodiments, a DNA damage pathway inhibitor is selected from the group consisting of bleomycin, an ATM inhibitor (e.g., AZD1390), a USP1 inhibitor, a WEE1 inhibitor (e.g., AZD1775), and a Chk1 inhibitor (e.g., AZD7762). In some embodiments, a DNA damage pathway inhibitor is a DNA alkylating agent.

In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a PARP inhibitor. In some embodiments, a PARP inhibitor is selected from the group consisting of olaparib, rucaparib, niraparib, talazoparib, veliparib, pamiparib, CEP 9722, E7016, iniparib, and 3-aminobenzamide.

Some patients may experience allergic reactions to the PRMT5 inhibitors described herein and/or other anti-cancer agent(s) during or after administration; therefore, anti-allergic agents are often administered to minimize the risk of an allergic reaction. In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an anti-allergic agent (e.g., a corticosteroid, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); an antihistamine, such as diphenhydramine (e.g., Benadryl®), hydroxyzine, and cyproheptadine; a bronchodilator, such as the beta-adrenergic receptor agonists, albuterol (e.g., Proventil®), and terbutaline (Brethine®)).

Some patients may experience nausea during and after administration of the PRMT5 inhibitors described herein and/or other anti-cancer agent(s); therefore, anti-emetics are used in preventing nausea (upper stomach) and vomiting. In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an anti-emetic (e.g., aprepitant (Emend®), ondansetron (Zofran®), granisetron HCl (Kytril®), lorazepam (Ativan®), dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and an analgesic (e.g., an over-the-counter analgesic (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), fentanyl (e.g., Duragesic®))).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a PRMT5 inhibitor as described herein, can be prepared and administered as described in the art, including, but not limited to, in the documents cited above.

In one embodiment, provided are pharmaceutical compositions comprising at least one compound of the present disclosure (e.g., a PRMT5 inhibitor, e.g., a compound of Formula (A) or Formula (I) to (III7a) or a compound of Table 1) or a pharmaceutically acceptable salt thereof together with a pharmaceutically acceptable carrier suitable for administration to a human or animal subject, either alone or together with other anti-cancer agents.

In one embodiment, provided are methods of treating human or animal subjects having or having been diagnosed with an MTAP-deficient and/or MTA accumulating proliferative disorder (e.g., cancer) comprising administering to the subject in need thereof a pharmaceutical composition comprising a therapeutically effective amount of a compound of the present disclosure (e.g., a compound of Formula (A) or Formula (I) to (III7a) or a compound of Table 1) or a pharmaceutically acceptable salt thereof in combination with one or more therapeutic agents as described herein.

In one embodiment, provided are methods of treating an MTAP-deficient and/or MTA accumulating proliferative disorder (e.g., cancer) in a subject in need thereof comprising administering to the subject a pharmaceutical composition comprising an effective amount (e.g., a therapeutically effective amount) of a compound of the present disclosure (e.g., a compound of Formula (A) or Formula (I) to (III7a) or a compound of Table 1) or a pharmaceutically acceptable salt thereof in combination with one or more therapeutic agents as described herein.

In particular, compositions will either be formulated together as a combination therapeutic or administered separately.

In combination therapy, a PRMT5 inhibitor as described herein and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure (e.g., a compound of Formula (A) or Formula (I) to (III7a) or a compound of Table 1, or pharmaceutically acceptable salts thereof) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The PRMT5 inhibitor as described herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another embodiment, provided are kits that include one or more PRMT5 inhibitor(s) as described herein (e.g., a compound of Formula (A) or Formula (I) to (III7a) or a compound of Table 1, or pharmaceutically acceptable salts thereof) and a second therapeutic agent as disclosed herein are provided. Representative kits include (a) a PRMT5 inhibitor as described herein or a pharmaceutically acceptable salt thereof (e.g., a compound of Formula (A) or Formula (I) to (III7a) or a compound of Table 1, or pharmaceutically acceptable salts thereof), (b) at least one other therapeutic agent, e.g., as indicated above, whereby such kit may comprise a package insert or other labeling including directions for administration.

A PRMT5 inhibitor as described herein (e.g., a compound of Formula (A) or Formula (I) to (III7a) or a compound of Table 1, or pharmaceutically acceptable salts thereof) may also be used in combination with known therapeutic processes, for example, the administration of hormones or especially radiation. A compound of the present disclosure may in particular be used as a radiosensitizer, especially for the treatment of tumors which exhibit poor sensitivity to radiotherapy. In some embodiments, provided is a method of treating a disease or disorder (e.g., cancer) comprising administering or coadministering, in any order, to a patient in need thereof a PRMT5 inhibitor described herein (e.g., a compound of Formula (A) or Formula (I) to (III7a) or a compound of Table 1, or pharmaceutically acceptable salts thereof) and radiation.

### Patient Selection and Monitoring

In one embodiment, provided is a method of determining if a subject having or having been diagnosed with a cancer (e.g., a cancer patient) will respond to therapeutic treatment with a PRMT5 inhibitor (e.g., an MTA-uncompetitive PRMT5 inhibitor, e.g., a compound of Formula (A) or Formula (I) to (III7a) or a compound of Table 1, or pharmaceutically acceptable salts thereof), comprising the steps of:
a) contacting a test sample obtained from said subject with a reagent capable of detecting human cancer cells that have MTAP deficiency and/or MTA accumulation; and
b) comparing the test sample with a reference (e.g., a reference sample taken from a non-cancerous or normal control subject),
wherein the presence of MTAP deficiency and/or MTA accumulation in said test sample indicates that the subject will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof).

In one embodiment, provided is a method of determining if a cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof), comprising the steps of:
a) contacting a test sample obtained from a subject having or having been diagnosed with said cancer with a reagent capable of detecting human cancer cells that have MTAP deficiency and/or MTA accumulation; and
b) comparing the test sample with a reference (e.g., a reference sample taken from a non-cancerous or normal control subject),
   wherein the presence of MTAP deficiency and/or MTA accumulation in said test sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof). In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In some embodiments, the method further comprises the step of determining the level of PRMT5 in the cancer cells. The level of expression of PRMT5 can be considered when determining the therapeutically effective dosage of a PRMT5 inhibitor.

In one embodiment, provided is a method of determining the sensitivity of a cancer cell to PRMT5 inhibition (e.g., inhibition with an MTA-uncompetitive PRMT5 inhibitor, e.g., a compound of Formula (A) or Formula (I) to (III7a) or a compound of Table 1, or pharmaceutically acceptable salts thereof), comprising the steps of:
a) assaying the production, level, activity, expression or presence of MTAP), in said cancer cell;
b) comparing the production, level, activity, expression or presence of MTAP in the cancer cell with the production, level, activity, expression or presence of MTAP, respectively, in a non-cancerous or normal control cell,
   wherein a decreased level, activity or expression in the cancer cell indicates MTAP deficiency and wherein MTAP deficiency indicates that said cancer cell is sensitive to the PRMT5 inhibitor.

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, provided is a method of determining the sensitivity of a cancer cell to a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof), comprising the steps of:
a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similarity in PRMT5 expression, and the presence of said MTAP deficiency in said cancer cell, indicates said cell is sensitive to a PRMT5 inhibitor.

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment the provided is a therapeutic method of treating a subject having or having been diagnosed with a cancer (e.g., a cancer associated with MTAP deficiency and/or MTA accumulation) comprising the steps of:
a) assessing the level of MTAP and/or MTA in a test sample obtained from said subject (e.g., by contacting the sample with a reagent capable of detecting human MTAP-deficient and/or MTA-accumulating cancer cells in a test sample obtained from said subject), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor; and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) to the subject identified in step b).

In one embodiment provided is a therapeutic method of treating a cancer (e.g., a cancer associated with MTAP deficiency and/or MTA accumulation) in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) to the subject identified in step b).

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a therapeutic method of treating a subject having or having been diagnosed with a cancer associated with MTAP deficiency and/or MTA accumulation comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) to the subject identified in step b).

In one embodiment provided is a therapeutic method of treating cancer associated with MTAP deficiency and/or MTA accumulation in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive PRMT5 inhibitor e.g., a compound of Formula (A) or Formula (I) to (III7a) or a compound of Table 1, or pharmaceutically acceptable salts thereof) to the subject identified in step b).

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a method of determining if a subject having or having been diagnosed with a cancer associated with MTAP deficiency and/or MTA accumulation will respond to treatment with a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor (e.g., an MTA-uncompetitive, non-competitive, or mixed mode PRMT5 inhibitor or an MTA-cooperative binding agent).

In one embodiment provided is a method of determining if a cancer associated with MTAP deficiency and/or MTA accumulation will respond to treatment with a PRMT5 inhibitor (e.g., an MTA-uncompetitive PRMT5 inhibitor, e.g., a compound of Formula (A) or Formula (I) to (III7a) or a compound of Table 1, or pharmaceutically acceptable salts thereof) comprising the steps of:
a) assessing the level of MTAP and/or MTA in a test sample obtained from a subject having or having been diagnosed with said cancer (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent).

In some embodiments, the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

### Sample Preparation

Further provided are assays for the detection of MTAP deficiency and/or MTA accumulation. They can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascites, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Detection of PRMT5 Selectivity

Samples, once prepared, can be tested for MTAP deficiency and/or MTA accumulation, either or both of which indicates that the sample is sensitive to treatment with a PRMT5 inhibitor. Cells can be determined to be MTA accumulating by techniques known in the art; methods for detecting MTA include, as a non-limiting example, liquid chromatography-electrospray ionization-tandem mass spectrometry (LC-ESI-MS/MS), as described in Stevens et al. 2010. J. Chromatogr. A. 1217:3282-3288; and Kirovski et al. 2011 Am. J. Pathol. 178:1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization, or amplification utilizing at least one probe or primer comprising a sequence of at least 12 contiguous nucleotides (nt) of the sequence of MTAP wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Radioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

### Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one embodiment, gene expression is detected and quantitated by hybridization to a probe that specifically hybridizes to the appropriate probe for that biomarker. The probes also can be attached to a solid support for use in high throughput screening assays using methods known in the art.

In one embodiment, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one embodiment, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Protein levels of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

### Adjacent Biomarkers

Near or adjacent to MTAP on chromosome 9 are several other biomarkers. CDKN2A is often, if not usually, deleted along with MTAP. Additional genes or pseudogenes in this region include: C9orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C9orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C9orf53, ERVFRD-3, TUBB8P1, KHSRPP1, MIR31, and MIR31HG.

Thus, in some embodiments, the disclosure encompasses: A method of determining if a subject having or having been diagnosed with a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor (e.g., an MTA-uncompetitive PRMT5 inhibitor, e.g., a compound of Formula (A) or Formula (I) to (III7a) or a compound of Table 1, or pharmaceutically acceptable salts thereof); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

In some embodiments, the disclosure encompasses: A method of determining if a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from a subject having or having been diagnosed with said cancer for MTAP deficiency, and evaluating a reference sample from a non-cancerous or normal control subject for MTAP deficiency, wherein MTAP deficiency in the test sample relative to the reference sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive PRMT5 inhibitor, e.g., a compound of Formula (A) or Formula (I) to (III7a) or a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

### Assaying for Biomarkers and PRMT5 Inhibitor Treatment

A number of patient stratification strategies could be employed to find patients likely to be sensitive to PRMT5 inhibition with an MTA-uncompetitive, non-competitive, or mixed mode PRMT5 inhibitor or an MTA-cooperative binding agent (e.g., a PRMT5 inhibitor of the present disclosure, e.g., a compound of Formula (A) or Formula (I) to (III7a) or a compound of Table 1, or pharmaceutically acceptable salts thereof), including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor (e.g., an MTA-uncompetitive, non-competitive, or mixed mode PRMT5 inhibitor or an MTA-cooperative binding agent, e.g., a compound of Formula (A) or Formula (I) to (III7a) or a compound of Table 1, or pharmaceutically acceptable salts thereof) to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

### Kits

In some embodiments provided are kits related to methods of use described herein.

In one embodiment, provided is a kit for predicting the sensitivity of a subject having or having been diagnosed with an MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

### EXAMPLES

In order that the invention(s) described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

In the synthetic schemes below, when the stereochemistry indicator (e.g., rac-, rel-) in the computer-generated name conflicts with the stereochemical indicators in the compound structure as depicted in the scheme, the stereochemical indicators in the scheme take precedence.

### Abbreviations

### General

ADDP 1,1′-(azodicarbonyl)dipiperidine
anhy. anhydrous
aq. aqueous
satd. saturated
min(s) minute(s)
hr(s) hour(s)
mL milliliter
mmol millimole(s)
mol mole(s)
MS mass spectrometry
NMR nuclear magnetic resonance
TLC thin layer chromatography
HPLC high-performance liquid chromatography
Me methyl
i-Pr iso-propyl
t-Bu tert-butyl
^{t}BuXPhos 2-di-tert-butylphosphino-2′,4′,6′-triisopropylbiphenyl
Ph phenyl
Et ethyl
Bz benzoyl
RuPhos 2-dicyclohexylphosphino-2′,6′-diisopropoxybiphenyl
Spectrum
Hz hertz
δ chemical shift
J coupling constant
S singlet
d doublet
t triplet
q quartet
m multiplet
br broad
qd quartet of doublets
dquin doublet of quintets
dd doublet of doublets
dt doublet of triplets

### Solvents and Reagents

DAST Diethylaminosulfurtrifluoride
CHCl₃ chloroform
DCM dichloromethane
DMF dimethylformamide
Et₂O diethyl ether
EtOH ethyl alcohol
EtOAc ethyl acetate
MeOH methyl alcohol
MeCN acetonitrile
PE petroleum ether
THF tetrahydrofuran
DMSO dimethyl sulfoxide
t-BuOK potassium tert-butoxide
9-BBN 9-borabicyclo[3.3.1]nonane
AcOH acetic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH₄ sodium borohydride
NaHCO₃ sodium bicarbonate
LiHMDS lithium hexamethyldisilylamide
NaBH₄ sodium borohydride
Et₃N triethylamine
Py pyridine
PCC pyridinium chlorochromate
DMAP 4-(dimethylamino)pyridine
DIPEA N,N-diisopropylethylamine
BINAP 2,2′-bis(diphenylphosphanyl)-1,1′-binaphthyl
dppf 1,1′-bis(diphenylphosphino) ferrocene
PEP Phospho (enol) pyruvic acid
LDH Lactate Dehydrogenase
DTT DL-Dithiothreitol
BSA Bovine Serum Albumin
NADH β-Nicotinamide adenine dinucleotide, reduced
Pd(t-Bu₃P)₂ bis(tri-tert-butylphosphine)palladium(0)
AcCl acetyl chloride
i-PrMgCl Isopropylmagnesium chloride
TBSCl tert-Butyl(chloro)dimethylsilane
(i-PrO)₄Ti titanium tetraisopropoxide
BHT 2,6-di-t-butyl-4-methylphenoxide
BzCl benzoyl chloride
CsF cesium fluoride
DCC dicyclohexylcarbodiimide
DMP Dess-Martin periodinane
EtMgBr ethylmagnesium bromide
EtOAc ethyl acetate
TEA triethylamine
AlaOH alanine
TBAF tetra-n-butylammonium fluoride
TBS t-butyldimethylsilyl
TMS trimethylsilyl
TMSCF₃ (Trifluoromethyl)trimethylsilane
Ts p-toluenesulfonyl
Bu butyl
Ti (OⁱPr)₄ tetraisopropoxytitanium
LAH Lithium Aluminium Hydride
LDA lithium diisopropylamide
LiOH·H₂O lithium hydroxide hydrates
MAD methyl aluminum bis(2,6-di-t-butyl-4-methylphenoxide)
NBS N-bromosuccinimide
Na₂SO₄ sodium sulfate
Na₂S₂O₃ sodium thiosulfate
PE petroleum ether
MeCN acetonitrile
Boc t-butoxycarbonyl
MTBE methyl tert-butyl ether
DIAD diisopropyl azodicarboxylate

### General Experimental Notes

In the following examples, the chemical reagents were purchased from commercial sources (such as Alfa, Acros, Sigma Aldrich, TCI and Shanghai Chemical Reagent Company), and used without further purification.

In some examples, purification of intermediates and final compounds was performed using HPLC (H₂O-MeOH; Agilent 1260 Infinity systems equipped with DAD and mass-detectors. Waters Sunfire C18 OBD Prep Column, 100 Å, 5 μm, 19 mm×100 mm with SunFire C18 Prep Guard Cartridge, 100 Å, 10 μm, 19 mm×10 mm). The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated under the N₂ flow upon heating to 80° C. On the basis of post-chromatography LCMS analysis fractions were united. Solid fractions were dissolved in 0.5 mL MeOH and transferred into pre-weighted marked vials. Obtained solutions were again evaporated under the N₂ flow upon heating to 80° C. After drying, products were subjected to lyophilization using acetonitrile-water mixtures and finally characterized by LCMS and ¹H NMR.

Nuclear magnetic resonance (NMR) spectra were recorded using Brucker AVANCE DRX 500, Bruker 400 spectrometer or Varian UNITYplus 400. Chemical shifts for protons were reported as parts per million in & scale using solvent residual peak (CHCl₃: 7.27 ppm) (methanol-d₄: 3.31 ppm)(DMSO-d₆: 2.50 ppm) or tetramethylsilane (0.00 ppm) as internal standards. Chemical shifts of ¹³C NMR spectra were reported in ppm from the central peak of CDCl₃ (77.00 ppm)(methanol-d₄: 49.15 ppm)(DMSO-d₆: 39.51 ppm) on the & scale. Data are represented as follows: chemical shift, multiplicity (s=singlet, d=doublet, t=triplet, q=quartet, qn=quintuplet, sx=sextet, sp=septuplet, m=multiplet, br=broad), coupling constant (J, Hz) and integration.

In certain examples, mass spectra were recorded on an Agilent 1100 Series LC/MSD system with DAD\ELSD and Agilent LC\MSD VL (G1956A), SL (G1956B) mass-spectrometer or an Agilent 1200 Series LC/MSD system with DAD\ELSD and Agilent LC\MSD SL (G6130 A), SL (G6140A) mass-spectrometer.

All the LC/MS data were obtained using positive/negative mode switching.
Column Zorbax SB-C18 1.8 μm 4.6×15 mm Rapid Resolution cartridge (PN 821975-932)
Mobile phase A—acetonitrile, 0.1% formic acid
   B—water (0.1% formic acid)
Flow rate 3 ml/min
Gradient 0 min-100% B
0.01 min-100% B
1.5 min-0% B
1.8 min-0% B
1.81 min-100% B
Injection volume 1 μl
Ionization mode atmospheric pressure chemical ionization (APCI)
Scan range m/z 80-1000.

### Other Exemplary Analytical LC/MS Instruments and Conditions are Described Below:

Instrument: Agilent LC1100-MS6100 series G1956B; Column: Xbridge Shield RP-18, 50*2.1 mm*5 μm; Mobile Phase A: H₂O with 0.05% NH₃—H₂O (v %); Mobile Phase B: MeCN; Flow rate: 1.0 mL/min; Wavelength: UV 220 nm, 254 nm; Column temperature: 30° C.; MS ionization: ESI.
0-30CD: Gradient: B from 0%˜30% over 2 minutes and holding at 30% for 0.48 minutes;
0-60CD: Gradient: B from 0%˜60% over 2 minutes and holding at 60% for 0.48 minutes;
10-80CD: Gradient: B from 10%˜80% over 2 minutes and holding at 80% for 0.48 minutes;
30-90CD: Gradient: B from 30%˜90% over 2 minutes and holding at 90% for 0.48 minutes;
50-100CD: Gradient: B from 50%˜100% over 2 minutes and holding at 100% for 0.48 minutes.

Instrument: Agilent LC1100-MS6100 series G1956B; Column: Xtimate C18, 30*2.1 mm*3 μm; Mobile Phase A: H₂O with 0.0375% TFA (v %); Mobile Phase B: MeCN with 0.01875% TFA (v %): Flow rate: 0.8 mL/min; Wavelength: UV 220 nm, 254 nm; Column temperature: 50° C.; MS ionization: ESI.
0-30AB: Gradient: B from 0%˜30% over 3 minutes and holding at 30% for 0.5 minutes;
0-60AB: Gradient: B from 0%˜60% over 3 minutes and holding at 30% for 0.5 minutes;
10-80AB: Gradient: B from 10%˜80% over 3 minutes and holding at 30% for 0.5 minutes;
30-90AB: Gradient: B from 0%˜30% over 3 minutes and holding at 30% for 0.5 minutes;
50-100AB: Gradient: B from 50%˜100% over 3 minutes and holding at 100% for 0.5 minutes.

Instrument: Shimadzu LC20-MS2010; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5˜95% over 0.7 minutes and holding at 95% for 0.4 minutes; Flow Rate: 1.5 mL/min; Wavelength: UV 220 nm, 254 nm, 215 nm; Column temperature: 50° C.; MS ionization: ESI.

Instrument: Shimadzu LC20-MS2020; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5˜95% over 0.7 minutes and holding at 95% for 0.4 minutes; Flow Rate: 1.5 mL/min; Wavelength: UV 220 nm, 254 nm; Column temperature: 50° C.; MS ionization: ESI.

### Exemplary HPLC Instruments and Conditions

Instrument: Shimadzu LC20; Column: YMC-Pack ODS-A 150*4.6 mm; Mobile
Phase A: H₂O with 0.06875% TFA (v %); Mobile Phase B: MeCN with 0.0625% TFA (v %);
Flow rate: 1.5 mL/min; Wavelength: UV 220 nm, 215 nm, 254 nm; Column temperature: 40° C.
0-30: Gradient: B from 0˜30% over 10 minutes and holding at 30% for 5 minutes;
0˜60: Gradient: B from 0˜60% over 10 minutes and holding at 60% for 5 minutes;
0-95: Gradient: B from 0˜95% over 10 minutes and holding at 95% for 5 minutes;
10-80: Gradient: B from 10˜80% over 10 minutes and holding at 80% for 5 minutes;
30-90: Gradient: B from 30˜90% over 10 minutes and holding at 90% for 5 minutes;
50-100: Gradient: B from 50˜100% over 10 minutes and holding at 100% for 5 minutes.

Instrument: Shimadzu LC20; Column: Xbridge Shield RP-18 50*2.1 mm, 5 μm; Mobile Phase A: H₂O with 0.01% NH₃—H₂O; Mobile Phase B: MeCN; Flow Rate: 1.2 mL/min; Wavelength: UV 220 nm, 215 nm, 254 nm; Column temperature: 40° C.
0-30CD: Gradient: B from 0˜30% over 6 minutes and holding at 30% for 2 minutes;
0-60CD: Gradient: B from 0˜60% over 6 minutes and holding at 60% for 2 minutes;
10-80CD: Gradient: B from 10˜80% over 6 minutes and holding at 80% for 2 minutes;
30-90CD: Gradient: B from 30˜90% over 6 minutes and holding at 90% for 2 minutes;
50-100CD: Gradient: B from 10˜80% over 6 minutes and holding at 100% for 2 minutes.

Instrument: Shimadzu LC20; Column: Ultimate C18 50*3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
0-30AB: Gradient: B from 0˜30% over 2.5 minutes and holding at 30% for 0.75 minutes;
0-60AB: Gradient: B from 0˜60% over 2.5 minutes and holding at 60% for 0.75 minutes;
5-95AB: Gradient: B from 5˜95% over 2.5 minutes and holding at 95% for 0.75 minutes.

Instrument: Shimadzu LC20; Column: Ultimate C18 50*3 mm, 3 μm; Mobile
Phase A: H₂O with 0.06875% TFA (v %); Mobile Phase B: MeCN with 0.0625% TFA (v %);
Flow Rate: 1.2 mL/min; Wavelength: UV 220 nm, 215 nm, 254 nm; Column temperature: 40° C.
10-80AB: Gradient: B from 10˜80% over 4 minutes and holding at 80% for 2 minutes.

### Exemplary TLC, Concentration and Normal Phase Chromatography.

Analytical thin layer chromatography (TLC) was performed with silica gel 60 F254 aluminum plates. Visualization was done under a UV lamp (254 nm) and by iodine or immersion in ethanolic phosphomolybdic acid (PMA) or potassium permanganate (KMnO₄), followed by heating using a heat gun. Organic solutions were concentrated by rotary evaporation at 20˜40° C. Purification of reaction products were generally done by flash column chromatography with 230-400 mesh silica gel or Agela flash silica column.

### Exemplary Chiral SFC Analytical Methods

Column: Chiralpak AD-3 150×4.6 mm I.D., 3 μm; Mobile phase: A: supercritical CO₂; Mobile phase B: EtOH (0.05% DEA); Gradient: from 5% to 40% of B in 5 min and hold 40% for 2.5 min, then 5% of B for 2.5 min; Flow rate: 2.5 mL/min; Column temperature: 35° C.; ABPR: 1500 psi.

Column: Chiralpak AD-3 100×4.6 mm I.D., 3 μm; Mobile phase: A: supercritical CO₂ Mobile phase B: EtOH (0.1% ethanolamine); Gradient: from 5% to 40% of B in 4.5 min and hold 40% for 2.5 min, then 5% of B for 1 min; Flow rate: 2.8 mL/min; Column temperature: 40° C.

### Exemplary Preparative HPLC Separation Methods

Basic condition (NH₃—H₂O): Instrument: Gilson GX-281 Liquid Handler, Gilson 322 Pump, Gilson 156 UV Detector; Column: Waters Xbridge 150×25 mm×5 μm; Mobile phase A: H₂O with 0.05% NH₃—H₂O (v %); Mobile phase B: MeCN; Gradient: B from 22% to 52% in 9.5 min, hold 100% B for 1 min; Flow Rate: 25 mL/min; Column Temperature: 30° C.; Wavelength: 220 nm, 254 nm.

Acid condition (HCOOH): Instrument: Gilson GX-281 Liquid Handler, Gilson 322 Pump, Gilson 156 UV Detector; Column: Agela Durashell C18 150*25 mm 5 μm; Mobile phase A: H₂O (0.0225% HCOOH); Mobile phase B: MeCN; Gradient: B from 7% to 37% in 9 min, hold 100% B for 0 min; Flow Rate: 25 mL/min; Column Temperature: 30° C.; Wavelength: 220 nm, 254 nm.

Acid condition (HCl): Gilson GX-281 Liquid Handler, Gilson 322 Pump, Gilson 156 UV Detector; Column: Xtimate C18 150*25 mm*5 μm; Mobile phase A: H₂O with 0.05% HCl (v %); Mobile phase B: MeCN; Gradient: B from 0% to 30% in 6.5 min, hold 100% B for 2.5 min; Flow Rate: 25 mL/min; Column Temperature: 30° C.; Wavelength: 220 nm, 254 nm).

Neutral condition (NH₄HCO₃): (Instrument: Gilson GX-281 Liquid Handler, Gilson 322 Pump, Gilson 156 UV Detector; Column: Waters Xbridge 150×25 mm×5 μm; Mobile phase A: H₂O with 10 mmol NH₄HCO₃; Mobile phase B: MeCN; Gradient: B from 39% to 69% in 10 min, hold 100% B for 2.5 min; Flow Rate: 25 mL/min; Column Temperature: 30° C.; Wavelength: 220 nm, 254 nm).

### Exemplary Large-Scale Separation

Basic condition: Instrument: Shimadzu LC-8A Pumps, Shimadzu SCL-10A VP System Controller, Shimadzu SPD-20AV UV/VIS Detector; Column: Phenomenex Gemini C18 250*50 mm*10 μm; Mobile phase A: water (0.04% NH₃-H₂O+10 mM NH₄HCO₃); Mobile phase B: MeCN; Gradient: B from 65% to 95% in 26 min, hold 100% B for 3 min; Flow Rate: 110 mL/min; Column Temperature: 30° C.; Wavelength: 220 nm, 254 nm.

Acid condition (TFA): Instrument: Shimadzu LC-20AP Pumps, Shimadzu CBM-20A System Controller Shimadzu SPD-20AV UV/VIS Detector; Column: Phenomenex luna C18 250×50 mm×10 μm; Mobile phase A: H₂O with 0.1% TFA (v %); Mobile phase B: MeCN; Gradient: B from 0% to 25% in 15 min, hold 100% B for 4 min; Flow Rate: 120 mL/min; Column Temperature: 30° C.; Wavelength: 220 nm, 254 nm.

### Exemplary Preparative Chiral SFC Method:

Exemplary chiral columns available for use in the separation/purification of the enantiomers/diastereomers provided herein include, but are not limited to, CHIRALPAK® AD-10, CHIRALCEL® OB, CHIRALCEL® OB—H, CHIRALCEL® OD, CHIRALCEL® OD-H, CHIRALCEL® OF, CHIRALCEL® OG, CHIRALCEL® OJ and CHIRALCEL® OK.

In certain examples, the chiral separation was performed under the following conditions: Instrument: Thar 80; Column: Daicel Chiralpak AD. 250×30 mm I.D. 10 μm; Mobile phase: supercritical CO₂/MeOH (0.1% NH₃—H₂O, v %)=60/40; Flow Rate: 70 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative pyrazoles that have been listed herein. The compounds provided herein may be prepared from known or commercially available starting materials and reagents by one skilled in the art of organic synthesis.

Exemplary general method for preparative HPLC: Column: Waters RBridge prep 10 μm C18, 19*250 mm. Mobile phase: acetonitrile, water (NH₄HCO₃)(30 L water, 24 g NH₄HCO₃, 30 mL NH₃·H₂O). Flow rate: 25 mL/min.

Exemplary general method for analytical HPLC: Mobile phase: A: water (10 mM NH₄HCO₃), B: acetonitrile Gradient: 5%-95% B in 1.6 or 2 min Flow rate: 1.8 or 2 mL/min; Column: XBridge C18, 4.6*50 mm, 3.5 μm at 45° C.

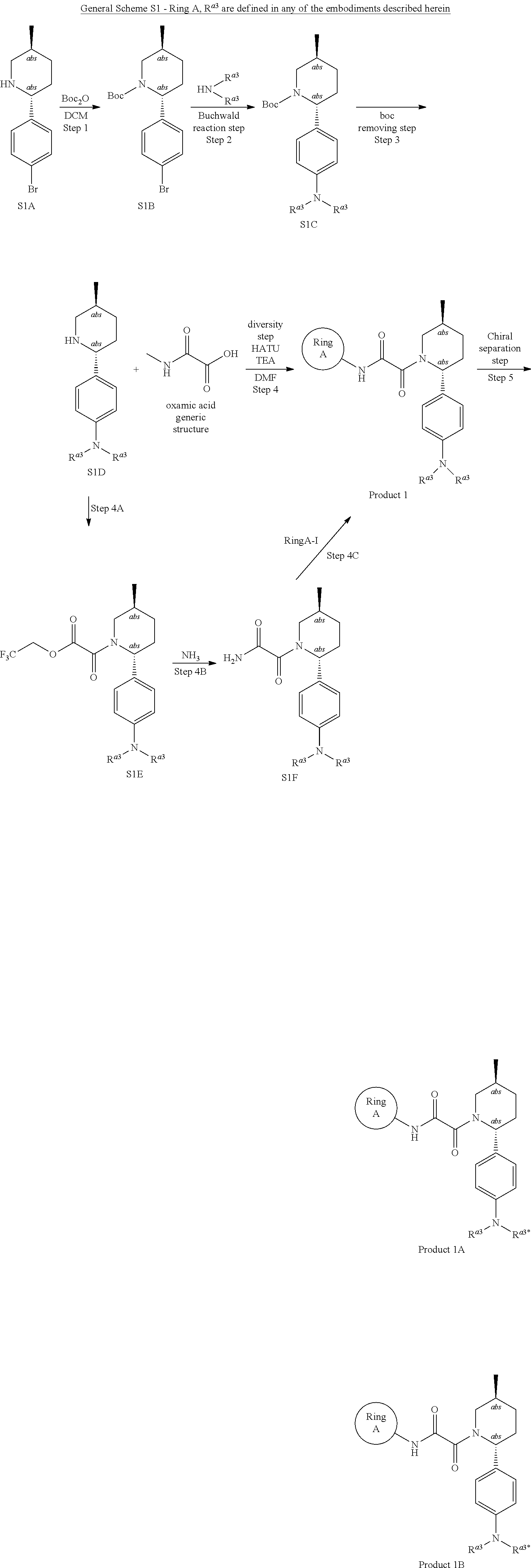

### Step 1: The Synthesis of SIB

S1A (1.0 equiv) was dissolved in DCM (280 mL) and a solution of di-tert-butyl dicarbonate (1.02 equiv) in DCM (20 mL) was added dropwise (vigorous gas evolution!). The resulting mixture was stirred at 20° C. for 4 hr. Then, the volatiles were removed under reduced pressure, affording crude product which was purified by FCC (CHCl₃-MeCN from 100%-0% to 0%-100%) to give tert-butyl (2R,5S)-2-(4-bromophenyl)-5-methyl-piperidine-1-carboxylate (S1B).

### Step 2: The Synthesis of SIC

To a stirred solution of tert-butyl (2R,5S)-2-(4-bromophenyl)-5-methyl-piperidine-1-carboxylate (S1B; 1.0 equiv) and respective amine (1.0 equiv) in Solvent (5 mL) was added Base (2.5 equiv). The resulting suspension was degassed with argon. Ligand (0.05 equiv) and Pd cat (0.05 eqiv) was added. The reaction mixture was stirred at 65 . . . 100° C. for 15 . . . 48 hr. After the completion of the reaction (monitored by LCMS), the resulting mixture was allowed to cool to the room temperature, filtered through a thin layer of SiO₂ and washed with solvent (2 mL). The volatiles were removed in vacuo and the residue was subjected for purification to afford S1C.

### Buchwald Reaction Step:

| | | | | Inert | | | | Purification |
|---|---|---|---|---|---|---|---|---|
| Method: | Pd cat | Base | Ligand | atm | Solvent | Temperature | Time | conditions |
| A | Pd₂(dba)₃ | Cs₂CO₃ | XanthPhos | Ar | dioxane | 100° C. | 16-48 h | Column |
| | | | | | | | | Chromatography |
| | | | | | | | | (chloroform- |
| | | | | | | | | MeOH 10:1) |
| B | Pd G4 | LiHMDS | RuPhos | Ar | THF | 65° C. | 18 h | Product was |
| | RuPhos | | | | | | | directly used in |
| | | | | | | | | the next step |
| C | Pd₂(dba)₃ | LiHMDS | RuPhos | Ar | THF | 65° C. | 18 h | HPLC |
| | | | | | | | | (60 - 60 - 100% |
| | | | | | | | | 0 - 1 - 6 min |
| | | | | | | | | H₂O/MeOH/0.1% |
| | | | | | | | | NH₄OH) |
| D | Pd₂(dba)₃ | Na t- | BrettPhos | Ar | dioxane | 100° C. | 16 h | column |
| | | butoxide | | | | | | chromatography |
| | | | | | | | | (CHCl₃-MeOH, |
| | | | | | | | | 10:1) or FCC |
| | | | | | | | | (MeOH in MTBE |
| | | | | | | | | from 0% to 100%) |
| E | Pd(OAc)₂ | Cs₂CO₃ | RuPhos | Ar | dioxane | 100° C. | 15 h | Product was |
| | | | | | | | | directly used in |
| | | | | | | | | the next step |
| F | Pd₂(dba)₃ | Cs₂CO₃ | BrettPhos | Ar | dioxane | 100° C. | 16 h | HPLC |
| | | | | | | | | chromatography |
| | | | | | | | | (65 - 65 - 80% |
| | | | | | | | | 0 - 1 - 6 min |
| | | | | | | | | H₂O/MeCN/0.1% |
| | | | | | | | | NH₄OH) |

### Step 3: The Synthesis of SID

S1C was dissolved in Solvent (2 . . . 10 mL) and Acid (50.0 equiv) was added. The reaction mixture was stirred at room temperature for 1 . . . 4 h. The solvent was removed in vacuo to obtain S1D.

### Boc Removing Step:

| | | | | | Work-up and |
|---|---|---|---|---|---|
| Method | Acid | Solvent | Temperature | Time | Purification |
| A | HCl | MeOH | RT | 4 h | The residue was |
| | (4.0M | | | | triturated with |
| | soln in | | | | MTBE—MeCN |
| | diox) | | | | mixture, filtered and |
| | | | | | the solvents were |
| | | | | | removed under |
| | | | | | reduced pressure. |
| B | TFA | DCM | RT | 1 h | The reaction mixture |
| | | | | | was carefully poured |
| | | | | | into K₂CO₃ solution |
| | | | | | (10 g of K₂CO₃ in |
| | | | | | 30 mL of water) and |
| | | | | | the resulting mixture |
| | | | | | was extracted with |
| | | | | | DCM (2*40 mL). |
| | | | | | Combined organic |
| | | | | | layers were washed |
| | | | | | with water (30 mL), |
| | | | | | dried over Na₂SO₄, |
| | | | | | filtered and |
| | | | | | concentrated in vacuo. |

### Step 4: The Synthesis of Product 1

S1D (1.0 equiv), oxamic acid (1.0 equiv) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuo and the residue was purified by HPLC to obtain Product 1.

### Step 4A: The Synthesis of S1E

S1D (1.0 equiv) and TEA (1.2 equiv) were dissolved in THF and cooled to 0° C., following by the dropwise addition of 2,2,2-trifluoroethyl 2-chloro-2-oxo-acetate (1.1 equiv) and the reaction mixture was stirred for 2 hr at 0° C. The mixture (S1E) was used in the next step without further purification.

### Step 4B: The Synthesis of S1E

Through a solution of S1E (1.0 equiv) in THF (50 mL), ammonia (50.0 equiv) was bubbled at 20° C. for 10 min. The mixture was stirred for 2 hr, evaporated in vacuo and triturated with MTBE-MeCN. The precipitate was filtered and dried to give S1F.

### Step 4C: The Synthesis of Product 1

S1F (1.0 equiv), Ring A-I (1.0 equiv), CuI (0.2 equiv), CuI (0.2 equiv), N¹,N²-dimethylcyclohexane-1,2-diamine (0.2 equiv) and Cs₂CO₃ were mixed together in dioxane (6 mL) under an Ar atmosphere. The reaction mixture was stirred at 100° C. for 48 h. Then, the mixture was allowed to cool to the room temperature and filtered off. The filtrate was subjected to HPLC to afford Product 1.

### Step 5: The Chiral Separation

Product 1 was chirally separated to obtain Product 1A and Product 1B.

### Example 1. The Synthesis of 3-(Dimethylamino)-N-[4-[(2R,5S)-1-[2-(imidazo[1,2-a]pyridin-7-ylamino)-2-oxo-acetyl]-5-methyl-2-piperidyl]phenyl]propanamide (Compound 52)

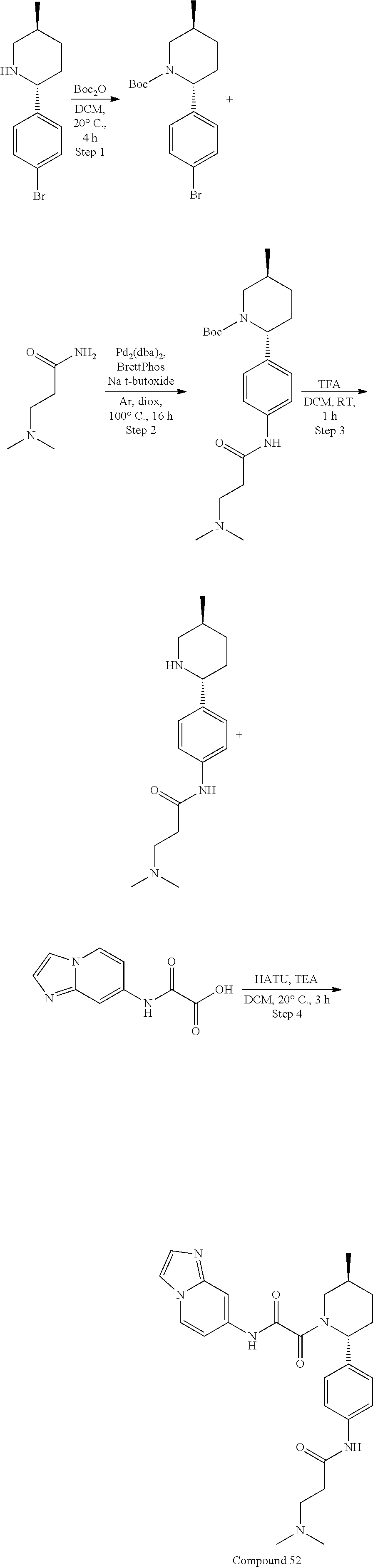

### Step 1: The Synthesis of tert-butyl (5S)-2-(4-bromophenyl)-5-methyl-piperidine-1-carboxylate

### Prepared by General Procedure Scheme S1 Step 1

Yield: 14 g (87.34%); LCMS(ESI): [M-Boc]⁺ m/z: calcd 254.0; found 254.0; Rt=1.539 min.

### Step 2: The Synthesis of tert-Butyl (2R,5S)-2-[4-[3-(dimethylamino) propanoylamino]phenyl]-5-methyl-piperidine-1-carboxylate

### Prepared by General Procedure Scheme S1 Step 2 (Method A)

Yield: 1.37 g (crude); LCMS(ESI): [M+H]⁺ m/z: calcd 390.2; found 390.2; Rt=1.154 min.

### Step 3: The Synthesis of 3-(Dimethylamino)-N-[4-[(2R,5S)-5-methyl-2-piperidyl]phenyl]propanamide

### Prepared by General Procedure Scheme S1 Step 3 (Method B)

Yield: 892.0 mg (87.95%); LCMS(ESI): [M+H]⁺ m/z: calcd 290.2; found 290.2; Rt=0.526 min.

### Step 4: The Synthesis of 3-(Dimethylamino)-N-[4-[(2R,5S)-1-[2-(imidazo[1,2-a]pyridin-7-ylamino)-2-oxo-acetyl]-5-methyl-2-piperidyl]phenyl]propanamide (Compound 52)

### Prepared by General Procedure Scheme S1 Step 4

Yield: 11.3 mg (2.29%).

HPLC conditions: 2-10 min 30-65% water-MeOH-0.1% NH₄OH; 30 mL/min; loading pump MeOH-0.1% NH₄OH 4 mL/min; column SunFire 19*100 mm

Compound 52: ¹H NMR (600 MHz, dmso) δ 0.97-1.04 (m, 3H), 1.30-1.37 (m, 1H), 1.65-1.72 (m, 1H), 1.84-2.23 (m, 9H), 2.40-2.42 (m, 2H), 2.53-2.55 (m, 2H), 3.22-3.24 (m, 1H), 3.43-4.01 (m, 1H), 5.11-5.57 (m, 1H), 7.03-7.09 (dd, 1H), 7.22-7.23 (d, 1H), 7.27-7.28 (d, 1H), 7.46-7.49 (d, 1H), 7.56-7.57 (d, 1H), 7.59-7.60 (d, 1H), 7.82-8.00 (m, 2H), 8.43-8.49 (dd, 1H), 10.01-10.04 (m, 1H), 11.03-11.10 (m, 1H). LCMS(ESI): [M+H]⁺ m/z: calcd 477.2; found 477.2; Rt=0.713 min.

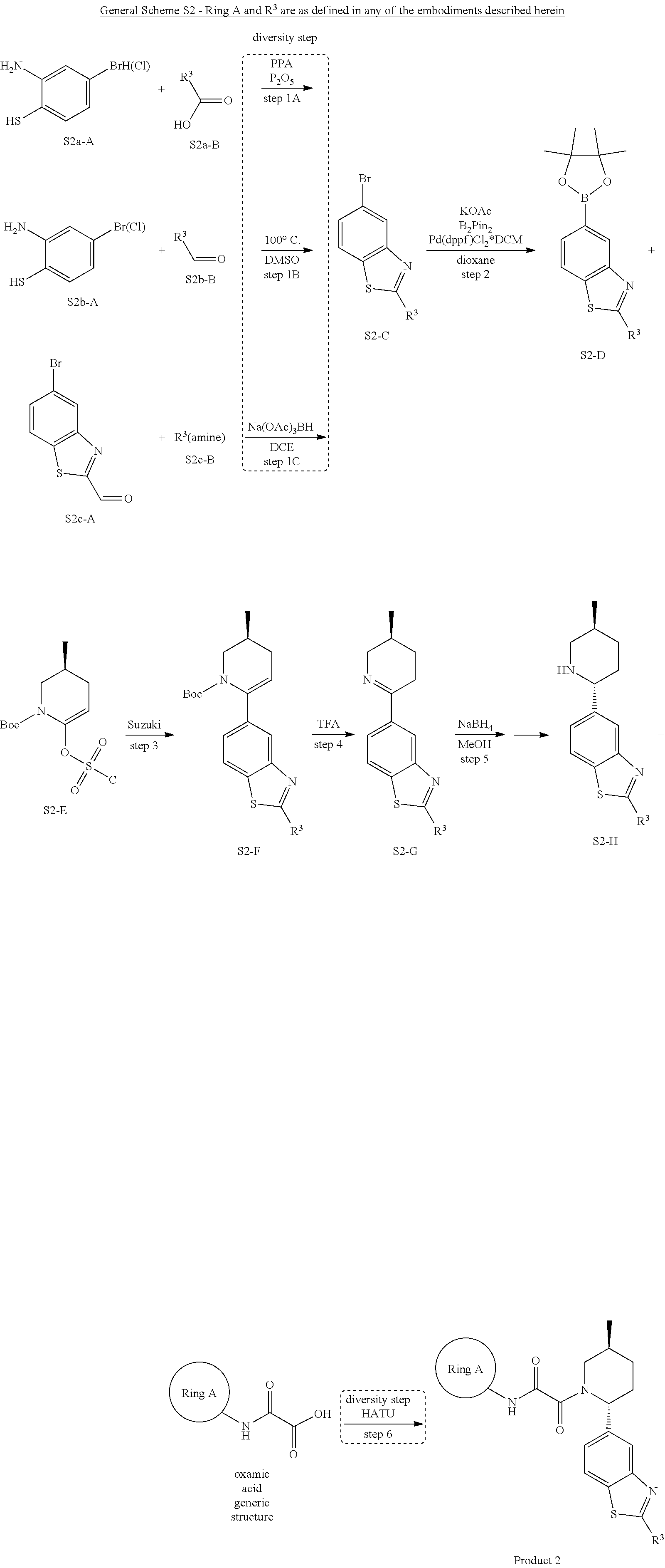

### Step 1A: Synthesis of S2-C

Phosphoric acid (4 eq) and phosphorus pentoxide (4 eq) were mixed together. The reaction suspension was stirred at rt for 10 min, then S2a-A (1 eq) followed by S2a-B (1.2 eq) were added under Ar. The solution was stirred at 110° C. for 18 hr then it was triturated with water, basidified (NaOH, 10% aq.) to pH=10, extracted with DCM twice, dried and evaporated in vacuum to give S2-C.

### Step 1B: Synthesis of S2-C

To the stirred solution of S2b-A (1 eq) in DMSO S2b-B (1 eq) was added. The resulting mixture was stirred at 100° C. for 14 hr. The reaction mixture was poured into cold water and extracted with MTBE twice. Combined organic layers were washed with water and brine, dried over Na₂SO₄. MTBE was evaporated in vacuum to give S2-C.

### Step 1C: Synthesis of S2-C

To the stirred solution of S2c-A (1 eq) in the 1,2-dichloroethane S2c-B (2 eq) was added and allowed to stir at 25° C. for 2 hr, sodium (trisacetoxy) borohydride (2 eq) was added. The reaction mixture was stirred at 25° C. for 16 hr. After completion, the reaction mixture was evaporated, quenched with water and neutralized by K₂CO₃ to pH=10. The aqueous phase was extracted with CHCl₃ twice. The combined organic phase was dried over Na₂SO₄ and evaporated under reduced pressure to afford S2-C. (TEA 1.5 eq per each acid eq, if amine salt used, was added to the solution of respective amine)

### Step 2: Synthesis of S2-D

S2-C (1 eq), B₂Pin₂ (1.1 eq) and KOAc (2 eq) were mixed in dioxane. The resulting mixture was evacuated and then backfilled with argon, this operation was repeated three times, then Pd(dppf)Cl₂*DCM (0.05 eq) was added under argon. The reaction mixture was stirred under argon at 90° C. for 14 hr, then cooled and filtered. The filter cake was washed with dioxane twice. The solvent was evaporated to afford S2-D.

### Step 3: Synthesis of S2-F

S2-D (1 eq), tert-butyl (3S)-3-methyl-6-(trifluoromethylsulfonyloxy)-3,4-dihydro-2H-pyridine-1-carboxylate (1.2 eq), sodium carbonate (3 eq) were mixed together in dioxane-water mixture (3:1). The resulting mixture was evacuated and then backfilled with argon. This operation was repeated two times, then Pd(dppf)Cl₂*DCM (819.86 mg, 1.00 mmol) was added and the reaction mixture was stirred under argon at 90° C. overnight, then cooled down and concentrated in vacuum. The residue was diluted with MTBE and stirred for 0.5 hr. After the most of the residue had dissolved, anhydrous sodium sulphate was added, and the resulting mixture was filtered. The filter cake was additionally washed with MTBE (5*50 ml) and discarded. The filtrate was concentrated in vacuum to afford S2-F.

### Step 4: Synthesis of S2-G

A solution of S2-F (1 eq) in TFA (15 eq) was stirred at rt for 1 hr, and then concentrated in vacuum. Cold water was added to the residue, and the resulting mixture was extracted with DCM twice. The DCM layer was discarded, and the aqueous layer was basified to pH 11. The resulting mixture was extracted with DCM twice. The combined organic extracts were dried over sodium sulphate and concentrated in vacuum to afford S2-G.

### Step 5: Synthesis of S2-H

S2-G (1 eq) was dissolved in MeOH and the resulting solution was cooled to 0° C. in an ice bath. Sodium borohydride (2 eq) was added portion wise to the previous solution. After addition completed, the reaction mixture was allowed to warm to rt and stirred overnight. Water was added to the reaction mixture and the resulting mixture was concentrated in vacuum. The residue was diluted with water and the resulting mixture was extracted with DCM twice, dried over Na₂SO₄, filtered and evaporated to obtain S2-H.

### Step 6A: Synthesis of Product 2

S2-H (1 eq), oxamic acid (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain Product 2.

### Step 6B: Synthesis of Product 2

DIPEA (2.5 eq+1.0 eq per each acid eq, if amine salt used) was added to the solution of respective amine or it salt (S2-H)(1 eq) and oxamic acid (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure Product 2.

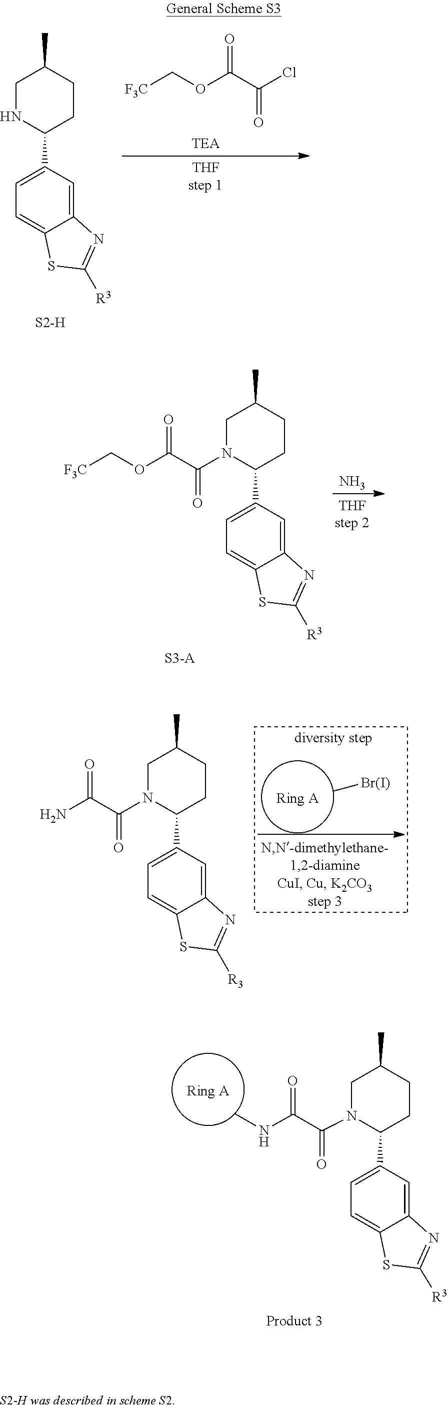

### Step 1: Synthesis of S3-A

S2-H (1 eq) and TEA (1.1 eq) were dissolved in THF and cooled to 0° C., following by the dropwise addition of 2,2,2-trifluoroethyl 2-chloro-2-oxo-acetate (1.1 eq) under Ar and the reaction mixture was stirred for 12 hr at rt and evaporated under reduced pressure to give S3-A which was used in the next step without further purification.

### Step 2: Synthesis of S3-B

To a solution of S3-A (1 eq) in THF (10 mL), ammonia (1 eq) was bubbled through for 10 min at 0° C. The reaction mixture was then stirred for 18 hr at rt. The reaction mixture was filtered off and the filtrate was evaporated in vacuum to give S3-B which was used in the next step without further purification.

### Step 3A: Synthesis of Product 3

S3-B (1 eq), Ring A-Br (1.1 eq), Cu (1 eq), CuI (1 eq), K₂CO₃ (2 eq) and N,N-dimethylcyclohexane-1,2-diamine (1.5 eq) were mixed in dioxane under argon, and then stirred overnight at 95° C. for 24 hr in vial. The residue was purified by HPLC to obtain pure Product 3.

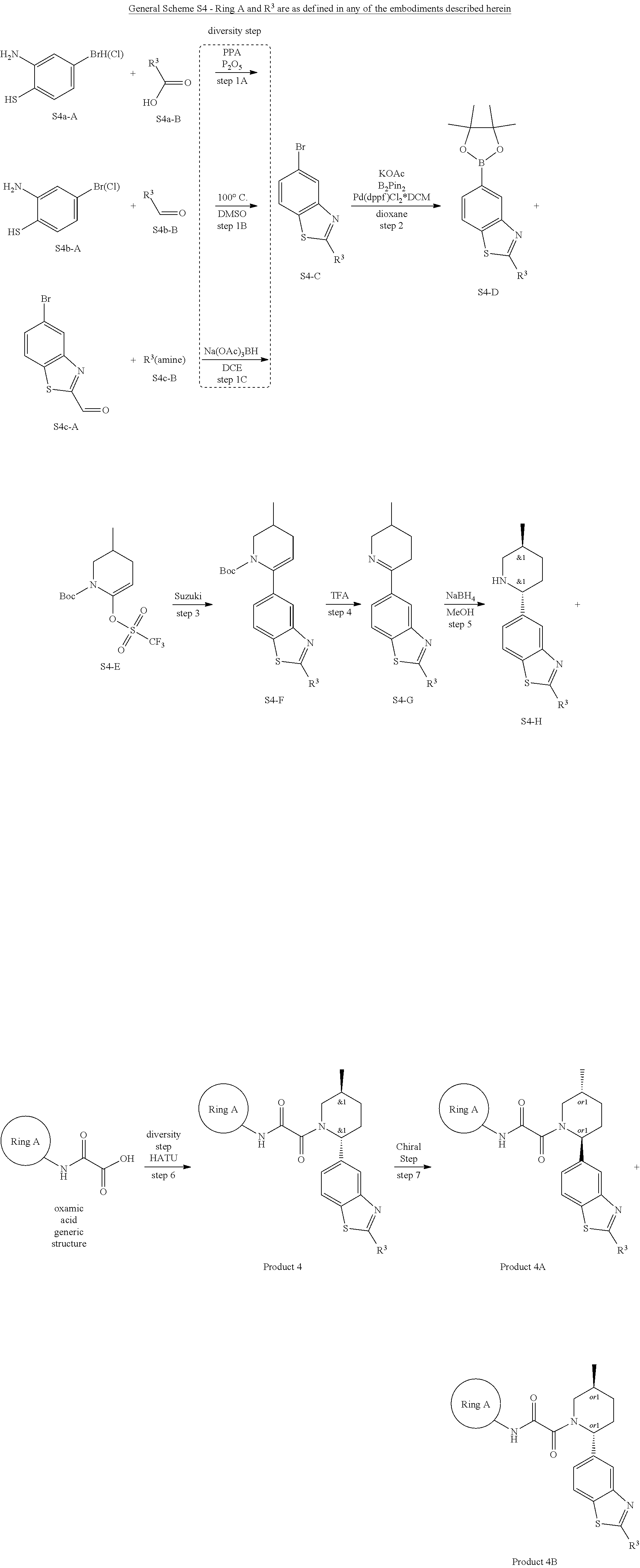

### Step 1A: Synthesis of S4-C

Phosphoric acid (4 eq) and phosphorus pentoxide (4 eq) were mixed together. The reaction suspension was stirred at rt for 10 min, then S4a-A (1 eq) followed by S4a-B (1.2 eq) were added under Ar. The solution was stirred at 110° C. for 18 hr then it was triturated with water, basified (NaOH, 10% aq.) to pH=10, extracted with DCM twice, dried and evaporated in vacuum to give S4-C.

### Step 1B: Synthesis of S4-C

To the stirred solution of S4b-A (1 eq) in DMSO S4b-B (1 eq) was added. The resulting mixture was stirred at 100° C. for 14 hr. The reaction mixture was poured into cold water and extracted with MTBE twice. Combined organic layers were washed with water and brine, dried over Na₂SO₄. MTBE was evaporated in vacuum to give S4-C.

### Step 1C: Synthesis of S4-C

To the stirred solution of S4c-A (1 eq) in the 1,2-dichloroethane S4c-B (2 eq) was added and allowed to stir at 25° C. for 2 hr, sodium (trisacetoxy) borohydride (2 eq) was added. The reaction mixture was stirred at 25° C. for 16 hr. After completion, the reaction mixture was evaporated, quenched with water and neutralized by K₂CO₃ to pH=10. The aqueous phase was extracted with CHCl₃ twice. The combined organic phase was dried over Na₂SO₄ and evaporated under reduced pressure to afford S4-C. (TEA 1.5 eq per each acid eq, if amine salt used, was added to the solution of respective amine)

### Step 2: Synthesis of S4-D

S4-C (1 eq), B₂Pin₂ (1.1 eq) and KOAc (2 eq) were mixed in dioxane. The resulting mixture was evacuated and then backfilled with argon, this operation was repeated three times, then Pd(dppf)Cl₂*DCM (0.05 eq) was added under argon. The reaction mixture was stirred under argon at 90° C. for 14 hr, then cooled and filtered. The filter cake was washed with dioxane twice. The solvent was evaporated to afford S4-D.

### Step 3: Synthesis of S4-F

S4-D (1 eq), tert-butyl 3-methyl-6-(trifluoromethylsulfonyloxy)-3,4-dihydro-2H-pyridine-1-carboxylate (1.2 eq), sodium carbonate (3 eq) were mixed together in dioxane-water mixture (3:1). The resulting mixture was evacuated and then backfilled with argon. This operation was repeated two times, then Pd(dppf)Cl₂*DCM (819.86 mg, 1.00 mmol) was added and the reaction mixture was stirred under argon at 90° C. overnight, then cooled down and concentrated in vacuum. The residue was diluted with MTBE and stirred for 0.5 hr. After the most of the residue had dissolved, anhydrous sodium sulphate was added, and the resulting mixture was filtered. The filter cake was additionally washed with MTBE (5*50 ml) and discarded. The filtrate was concentrated in vacuum to afford S4-F.

### Step 4: Synthesis of S4-G

A solution of S4-F (1 eq) in TFA (15 eq) was stirred at rt for 1 hr, and then concentrated in vacuum. Cold water was added to the residue, and the resulting mixture was extracted with DCM twice. The DCM layer was discarded, and the aqueous layer was basified to pH 11. The resulting mixture was extracted with DCM twice. The combined organic extracts were dried over sodium sulphate and concentrated in vacuum to afford S4-G.

### Step 5: Synthesis of S4-H

S4-G (1 eq) was dissolved in MeOH and the resulting solution was cooled to 0° C. in an ice bath. Sodium borohydride (2 eq) was added portion wise to the previous solution. After addition completed, the reaction mixture was allowed to warm to rt and stirred overnight. Water was added to the reaction mixture and the resulting mixture was concentrated in vacuum. The residue was diluted with water and the resulting mixture was extracted with DCM twice, dried over Na₂SO₄, filtered and evaporated to obtain S4-H.

### Step 6A: Synthesis of Product 4

S4-H (1 eq), oxamic acid (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain Product 4.

### Step 6B: Synthesis of Product 4

DIPEA (2.5 eq+1.0 eq per each acid eq, if amine salt used) was added to the solution of respective amine or it salt (S4-H)(1 eq) and oxamic acid (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure product Product 4.

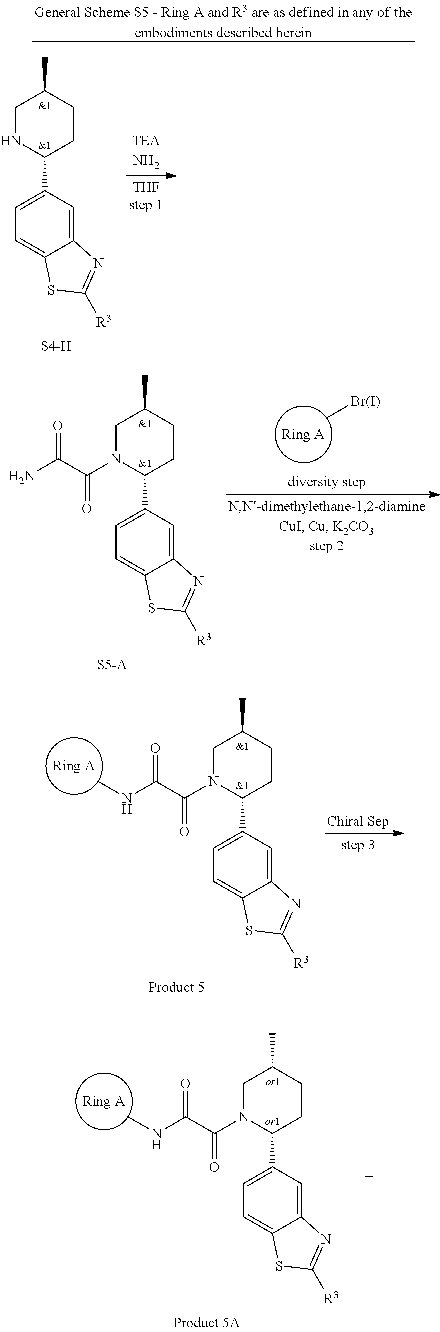

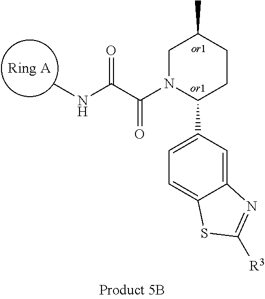

### Step 1: Synthesis of S5-A

S4-H was described in scheme S4.

To a solution of S4-H (1 eq) and TEA (1.2 eq) in THF was added 2,2,2-trifluoroethyl 2-chloro-2-oxo-acetate (1.1 eq) dropwise at 0° C. under argon. The reaction mixture was then stirred for 5 hr at rt, then was added Ammonium (20 eq). Resulting solution was stirred at 20° C. for 5 hr. Then, volatiles were removed under reduced pressure, leaving S5-A.

### Step 2A: Synthesis of Product 5

S5-A (1 eq), R³Br (1 eq), Cu (0.1 eq), CuI (1 eq), K₂CO₃ (2 eq) and N,N-dimethylethanediamine (0.1 eq) were mixed in toluene under argon, and then stirred overnight at 100° C. for 12 hr. This mixture was filtered and toluene was evaporated in vacuum. The residue was purified by HPLC to obtain pure product Product 5.

### Example 2. Synthesis of N-(imidazo[1,2-a]pyridin-7-yl)-2-((2R,5S)-5-methyl-2-(2-(1-methylpiperidin-3-yl)benzo[d]thiazol-5-yl)piperidin-1-yl)-2-oxoacetamide (Compound 33)

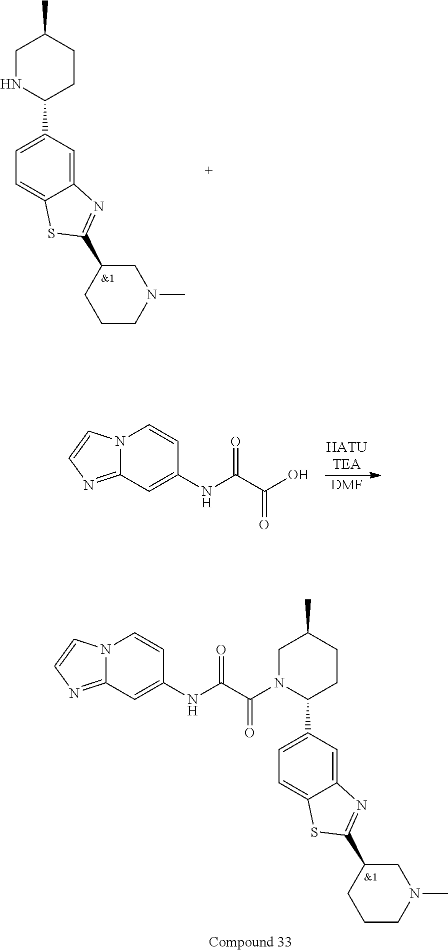

Synthesis of 5-((2R,5S)-5-methylpiperidin-2-yl)-2-(rac-(R)-1-methylpiperidin-3-yl)benzo[d]thiazole is given in Example 35.

Prepared by general procedure scheme S2 step 6A. Yield: 58 mg (51.86%).

HPLC conditions: Column: XBridge BEH C18 100*19 mm, 5 microM; 0-5 min 50-100% water-MeOH+0.1% NH₄OH; (loading pump 4 ml/min MeOH).

Compound 33: ¹H NMR (500 MHZ, DMSO-d₆) δ (ppm) 1.04 (m, 3H), 1.36 (m, 1H), 1.59 (m, 2H), 1.72 (m, 2H), 1.89 (m, 1H), 2.07 (m, 3H), 2.21 (m, 3H), 2.29 (s, 2H), 2.64 (m, 2H), 2.97 (m, 2H), 3.87 (m, 1H), 5.50 (m, 1H), 7.05 (m, 1H), 7.42 (m, 2H), 7.85 (m, 2H), 8.05 (m, 2H), 8.45 (m, 1H), 11.17 (s, 1H). LCMS(ESI): [M]⁺ m/z: calcd 516.2; found 517.2; Rt=2.032 min.

### Example 3. Synthesis of 2-((2R,5S)-2-(2-(1-(dimethylamino) propan-2-yl)benzo[d]thiazol-5-yl)-5-methylpiperidin-1-yl)-2-oxo-N-(1H-pyrazolo[4,3-c]pyridin-7-yl)acetamide (Compound 21)

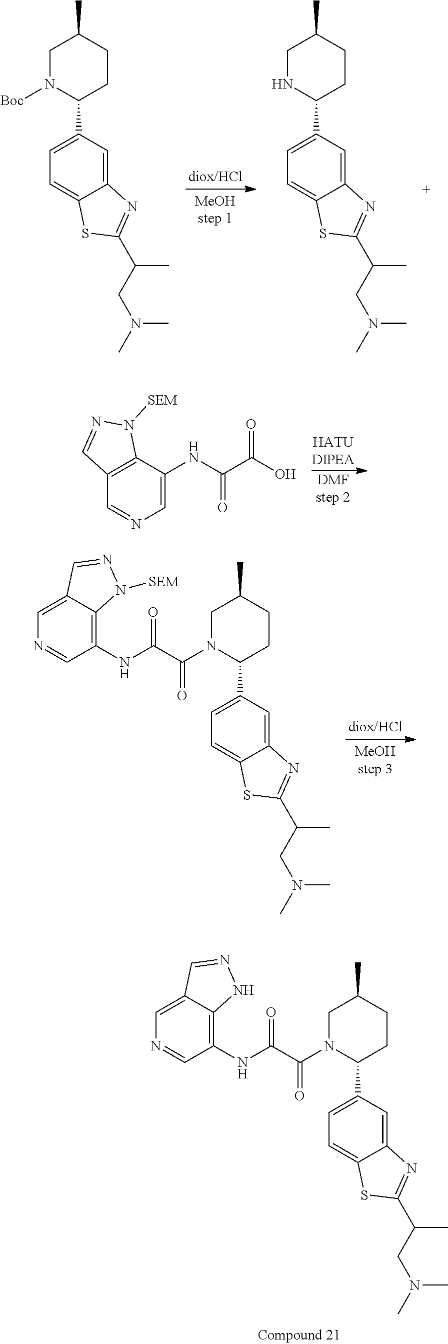

### Step 1: Synthesis of N,N-dimethyl-2-(5-((2R,5S)-5-methylpiperidin-2-yl)benzo[d]thiazol-2-yl) propan-1-amine

tert-Butyl (2R,5S)-5-methyl-2-[2-[(1S)-2-(dimethylamino)-1-methyl-ethyl]-1,3-benzothiazol-5-yl]piperidine-1-carboxylate (0.55 g, 1.32 mmol) was stirred in MeOH (10 mL) and diox/HCl (5 mL) for 16 hr at 25° C. Upon completion, the reaction mixture was evaporated, the crude product was quenched with water (20 mL) and neutralized by NaHCO₃ to pH-8. The aqueous phase was extracted with CHCl₃ (2*20 mL). The combined organic phase was dried over Na₂SO₄ and concentrated under reduced pressure. The N,N-dimethyl-2-[5-[(2R,5S)-5-methyl-2-piperidyl]-1,3-benzothiazol-2-yl]propan-1-amine (0.4 g, 1.26 mmol, 95.66% yield) was isolated.

The compound was obtained as racemate as a result of complete racemization during HCl deprotection step. LCMS(ESI): [M]⁺ m/z: calcd 317.2; found 318.2; Rt=2.652 min.

### Step 2: Synthesis of 2-((2R,5S)-2-(2-(1-(dimethylamino) propan-2-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6B. Yield: 100 mg of crude.

LCMS(ESI): [M]⁺ m/z: calcd 635.2; found 636.2; Rt=1.294 min.

### Step 3: Synthesis of 2-((2R,5S)-2-(2-(1-(dimethylamino) propan-2-yl)benzo[d]thiazol-5-yl)-5-methylpiperidin-1-yl)-2-oxo-N-(1H-pyrazolo[4,3-c]pyridin-7-yl)acetamide (Compound 21)

The solution of 2-oxo-2-[(2R,5S)-5-methyl-2-[2-[-2-(dimethylamino)-1-methyl-ethyl]-1,3-benzothiazol-5-yl]-1-piperidyl]-N-[1-(2-trimethylsilylethoxymethyl)pyrazolo[4,3-c]pyridin-7-yl]acetamide (100 mg, 157.26 μmol) in MeOH (5 mL) and diox/HCl (5 mL) was stirred at 25° C. for 16 hr. Upon completion, the reaction mixture was concentrated under reduced pressure to obtain crude product. The obtained crude product was purified by reverse phase HPLC chromatography (Device (Mobile Phase, Column): SYSTEM 5-5-40% 0-2-5 min H₂O/MeCN/0.2% FA, flow: 30 ml/min (loading pump 4 ml/min MeCN) target mass 505 column: Chromatorex 18 SMB 100-5T 100×19 mm 5 um) to afford product 2-oxo-N-(1H-pyrazolo[4,3-c]pyridin-7-yl)-2-[(2R,5S)-5-methyl-2-[2-[2-(dimethylamino)-1-methyl-ethyl]-1,3-benzothiazol-5-yl]-1-piperidyl]acetamide (25.3 mg, 31.81% yield). After purification the analysis of Enantiomeric excess was performed, the result of ee=0 was obtained.

Compound 21: ¹H NMR (600 MHz, DMSO-d₆) δ (ppm) 1.05-1.13 (m, 3H), 1.43-1.47 (m, 3H), 1.65-1.75 (m, 1H), 1.84-2.00 (m, 1H), 2.12-2.35 (m, 2H), 2.83-2.87 (m, 6H), 2.89-2.97 (m, 1H), 3.69-3.80 (m, 2H), 3.89-4.12 (m, 2H), 5.40-5.78 (m, 1H), 7.47-7.53 (m, 1H), 7.93-8.02 (m, 1H), 8.09-8.19 (m, 1H), 8.67-8.79 (m, 1H), 8.84-8.99 (m, 1H), 9.34-9.47 (m, 1H), 9.47-9.56 (m, 1H), 11.66-12.06 (m, 1H), 14.37 (br s, 1H).

LCMS(ESI): [M]⁺ m/z: calcd 505.2; found 506.2; Rt=2.097 min.

### Example 4. Synthesis of 2-((2R,5S)-2-(2-(2-(dimethylamino)ethyl)benzo[d]thiazol-5-yl)-5-methylpiperidin-1-yl)-N-(3-methylimidazo[1,2-a]pyridin-7-yl)-2-oxoacetamide (Compound 34)

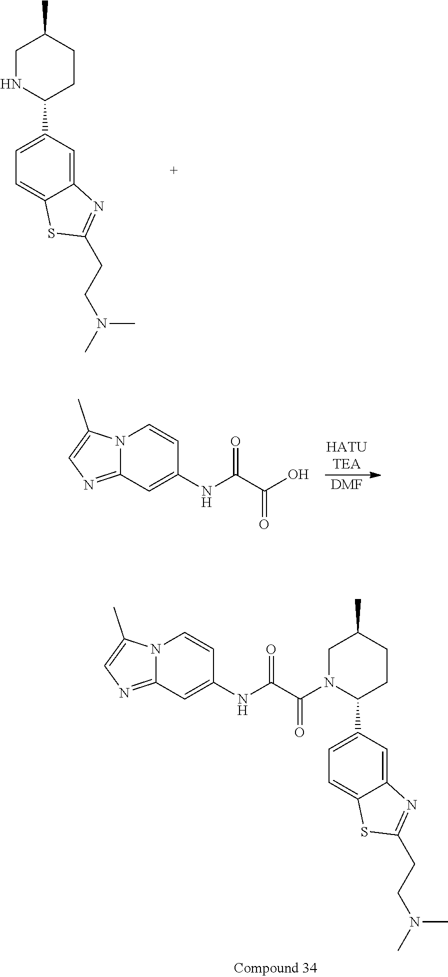

The synthesis of N,N-dimethyl-2-[5-[(2R,5S)-5-methyl-2-piperidyl]-1,3-benzothiazol-2-yl]ethanamine is given in Intermediate 1.

Prepared by general procedure scheme S2 step 6A using Intermediate 34. Yield: 99 mg (29.77%).

HPLC conditions: Column: YMC Triart C18 100*20 mm, 5 microM; 0-5 min 40-85% water-MeOH+0.1% NH₄OH, flow: 30 ml/min; (loading pump 4 ml/min MeOH).

Compound 34: ¹H NMR (DMSO-d6, 600 MHZ): δ (ppm) 1.05 (m, 3H), 1.35 (m, 1H), 1.72 (m, 1H), 1.89 (m, 1H), 2.11 (m, 1H), 2.20 (m, 7H), 2.30 (m, 1H), 2.40 (m, 3H), 2.68 (m, 2H), 3.21 (m, 3H), 3.86 (m, 1H), 5.51 (m, 1H), 7.11 (m, 1H), 7.26 (m, 1H), 7.38 (m, 1H), 7.86 (m, 1H), 7.94 (m, 1H), 8.03 (m, 1H), 8.20 (m, 1H), 11.11 (m, 1H). LCMS(ESI): [M]⁺ m/z: calcd 504.2; found 505.2; Rt=1.793 min.

### Example 5. Synthesis of 2-oxo-N-(5-oxo-6,7-dihydropyrrolo[3,4-b]pyridin-3-yl)-2-[(2R,5S)-5-methyl-2-[2-(1-methyl-4-piperidyl)-1,3-benzothiazol-5-yl]-1-piperidyl]acetamide (Compound 87)

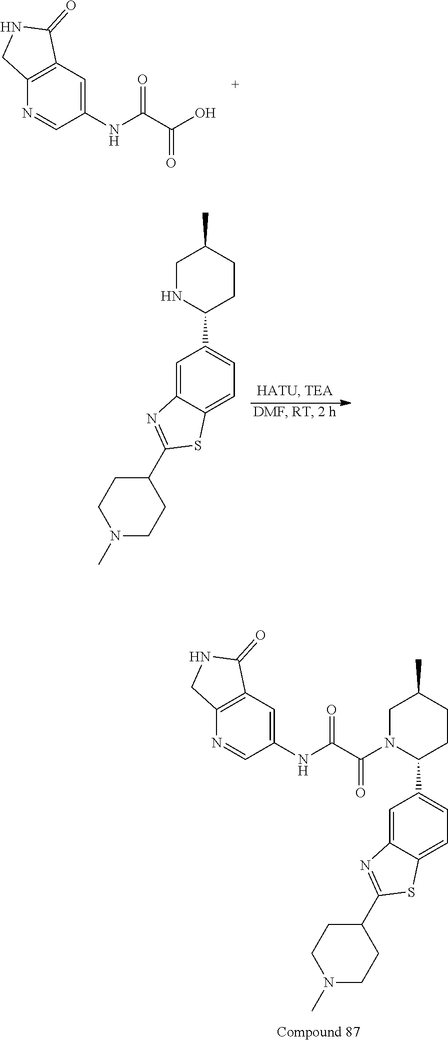

The synthesis of amine 5-((2R,5S)-5-methylpiperidin-2-yl)-2-(1-methylpiperidin-4-yl)benzo[d]thiazole is given in Intermediate 2.

The synthesis of 2-oxo-2-[(5-oxo-6,7-dihydropyrrolo[3,4-b]pyridin-3-yl)amino]acetic acid is given in Intermediate 35.

Prepared by general procedure scheme S2 step 6A. Yield: 8.0 mg (3.32%).

HPLC conditions: 30-80% 0-5 min H₂O/MeOH/0.1% NH₄OH, flow: 30 ml/min (loading pump 4 ml/min methanol) target mass 532.67 column: XBridge C18 100×19 mm, 5 um.

Compound 87: ¹H NMR (600 MHz, DMSO-d₆) δ (ppm) 1.02-1.07 (m, 3H), 1.31-1.40 (m, 1H), 1.70-1.92 (m, 4H), 2.00-2.11 (m, 5H), 2.18 (s, 3H), 2.28-2.34 (m, 1H), 2.79-2.86 (m, 2H), 2.88-3.09 (m, 2H), 3.51-4.04 (m, 1H), 4.30-4.42 (m, 2H), 5.30-5.75 (m, 1H), 7.34-7.44 (m, 1H), 7.86-7.91 (m, 1H), 7.99-8.10 (m, 1H), 8.24-8.42 (m, 1H), 8.77-8.84 (m, 1H), 8.84-8.96 (m, 1H), 11.19-11.54 (m, 1H). LCMS(ESI): [M+1]⁺ m/z: calcd 533.2; found 533.2; Rt=2.404 min.

### Example 6. Synthesis of 2-((2R,5S)-2-(2-(1-cyclopropylpiperidin-4-yl)benzo[d]thiazol-5-yl)-5-methylpiperidin-1-yl)-2-oxo-N-(5-oxo-5,6,7,8-tetrahydro-1,6-naphthyridin-3-yl)acetamide (Compound 4)

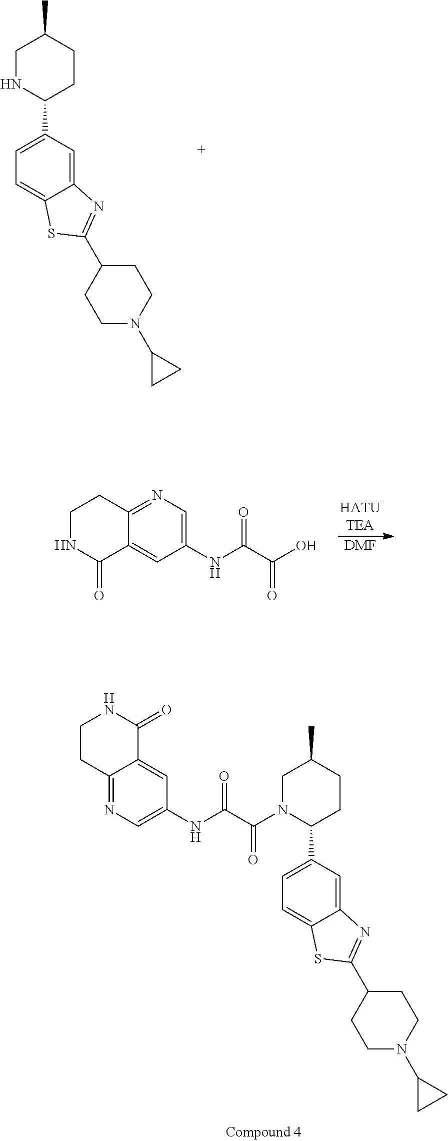

The synthesis of 2-(1-cyclopropylpiperidin-4-yl)-5-((2R,5S)-5-methylpiperidin-2-yl)benzo[d]thiazole is given in Intermediate 3.

Synthesis of 2-oxo-2-((5-oxo-5,6,7,8-tetrahydro-1,6-naphthyridin-3-yl)amino) acetic acid is given in Intermediate 36.

Prepared by general procedure scheme S2 step 6A. Yield: 23 mg (9.07%).

HPLC conditions: Column: YMC Triart C18 100*20 mm, 5 microM; 0-1-6 min 40-40-80% water-MeOH+0.1% NH₄OH; (loading pump 4 ml/min MeOH).

Compound 4: ¹H NMR (600 MHZ, DMSO-d₆) δ (ppm) 0.23-0.36 (m, 2H), 0.36-0.46 (m, 2H), 0.97-1.09 (m, 3H), 1.30-1.43 (m, 1H), 1.59-1.64 (m, 1H), 1.65-1.79 (m, 3H), 1.83-1.97 (m, 1H), 2.00-2.24 (m, 3H), 2.27-2.37 (m, 3H), 2.84-2.98 (m, 2H), 2.98-3.04 (m, 3H), 3.06-3.15 (m, 1H), 3.39-3.46 (m, 2H), 3.50-4.06 (m, 1H), 5.20-5.77 (m, 1H), 7.29-7.45 (m, 1H), 7.84-7.89 (m, 1H), 8.00-8.08 (m, 1H), 8.08-8.16 (m, 1H), 8.36-8.56 (m, 1H), 8.74-9.02 (m, 1H), 11.13-11.32 (m, 1H). LCMS(ESI): [M+1]⁺ m/z: calcd 572.2; found 573.2; Rt=2.623 min.

### Example 7. The synthesis of 2-((2R,5S)-2-(2-(3-((dimethylamino)methyl) oxetan-3-yl)benzo[d]thiazol-5-yl)-5-methylpiperidin-1-yl)-N-(imidazo[1,2-a]pyridin-7-yl)-2-oxoacetamide (Compound 96)

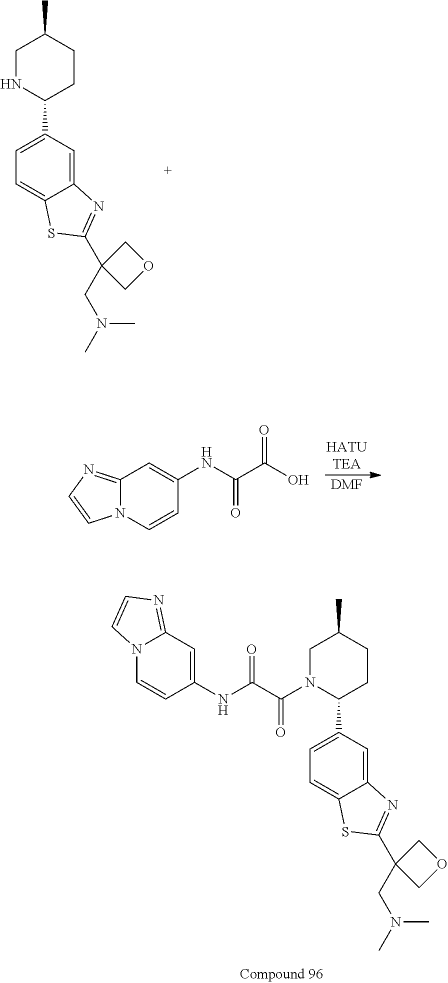

The synthesis of N,N-dimethyl-1-(3-(5-((2R,5S)-5-methylpiperidin-2-yl)benzo[d]thiazol-2-yl) oxetan-3-yl) methanamine is given in Intermediate 4.

Prepared by general procedure scheme S2 step 6A. Yield: 31.6 mg (13.66%).

HPLC conditions: Column: YMC Triart C18 100*20 mm, 5 microM; 0-1-5 min 20-60% water-MeCN+0.1% NH₄OH; (loading pump 4 ml/min MeOH).

Compound 96: ¹H NMR (600 MHz, DMSO) δ 1.02-1.09 (m, 3H), 1.31-1.44 (m, 1H), 1.70-1.78 (m, 1H), 1.85-1.95 (m, 1H), 2.05-2.10 (m, 6H), 2.10-2.36 (m, 3H), 2.88-3.39 (m, 3H), 3.48-4.09 (m, 1H), 4.70-4.98 (m, 4H), 5.29-5.75 (m, 1H), 7.00-7.12 (m, 1H), 7.38-7.51 (m, 2H), 7.79-7.88 (m, 1H), 7.88-7.97 (m, 1H), 7.97-8.04 (m, 1H), 8.04-8.13 (m, 1H), 8.40-8.52 (m, 1H), 11.05-11.27 (m, 1H). LCMS(ESI): [M]⁺ m/z: calcd 532.2; found 533.2; Rt=2.117 min.

### Example 8. The synthesis of N-(imidazo[1,2-a]pyridin-7-yl)-2-((2R,5S)-5-methyl-2-(2-(1-(pyrrolidin-1-yl) propan-2-yl)benzo[d]thiazol-5-yl)piperidin-1-yl)-2-oxoacetamide (Compound 32)

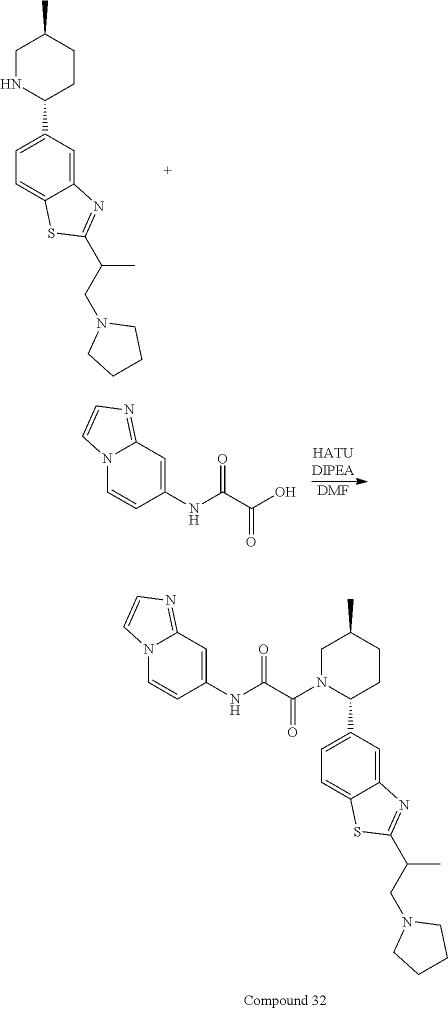

The Synthesis of 5-((2R,5S)-5-methylpiperidin-2-yl)-2-(1-(pyrrolidin-1-yl) propan-2-yl)benzo[d]thiazole is given in Example 22.

5-[(2R,5S)-5-Methyl-2-piperidyl]-2-[1-methyl-2-pyrrolidin-1-yl-ethyl]-1,3-benzothiazole (198.83 mg, 578.80 μmol), 2-(imidazo[1,2-a]pyridin-7-ylamino)-2-oxo-acetic acid (118.75 mg, 578.80 μmol) and DIPEA (224.42 mg, 1.74 mmol, 302.45 μL) were dissolved in the DMF (8 mL) and stirred 5 min. Then HATU (286.10 mg, 752.44 μmol) was added to reaction mixture and the reaction was stirred at 25° C. for 12 hr. After completion the solvent was evaporated under reduced pressure and residue was purified by HPLC (Device (Mobile Phase, Column): SYSTEM 60-60-75% 0-1-5 min H₂O/MeOH/0.1% NH₄OH, flow: 30 ml/min (loading pump 4 ml/min MeOH) column: XBridge BEH C18 100×19 mm, 5 um) to give N-imidazo[1,2-a]pyridin-7-yl-2-oxo-2-[(2R,5S)-5-methyl-2-[2-[1-methyl-2-pyrrolidin-1-yl-ethyl]-1,3-benzothiazol-5-yl]-1-piperidyl]acetamide (85.9 mg, 161.87 μmol, 27.96%).

Compound 32: ¹H NMR (DMSO-d6, 500 MHz): δ (ppm) 1.05 (m, 3H), 1.37 (m, 4H), 1.66 (m, 5H), 1.89 (m, 1H), 2.20 (m, 3H), 2.52 (m, 3H), 2.77 (m, 3H), 3.57 (m, 2H), 5.50 (d, 1H), 7.06 (m, 1H), 7.41 (m, 2H), 7.96 (m, 4H), 8.46 (m, 1H), 11.14 (m, 1H). LCMS(ESI): [M]⁺ m/z: calcd 530.2; found 531.2; Rt=2.120 min.

### Example 9. Synthesis of 2-((2R,5S)-5-methyl-2-(2-(2-(pyrrolidin-1-yl)ethyl)benzo[d]thiazol-5-yl)piperidin-1-yl)-N-(3-methylimidazo[1,2-a]pyridin-7-yl)-2-oxoacetamide (Compound 24)

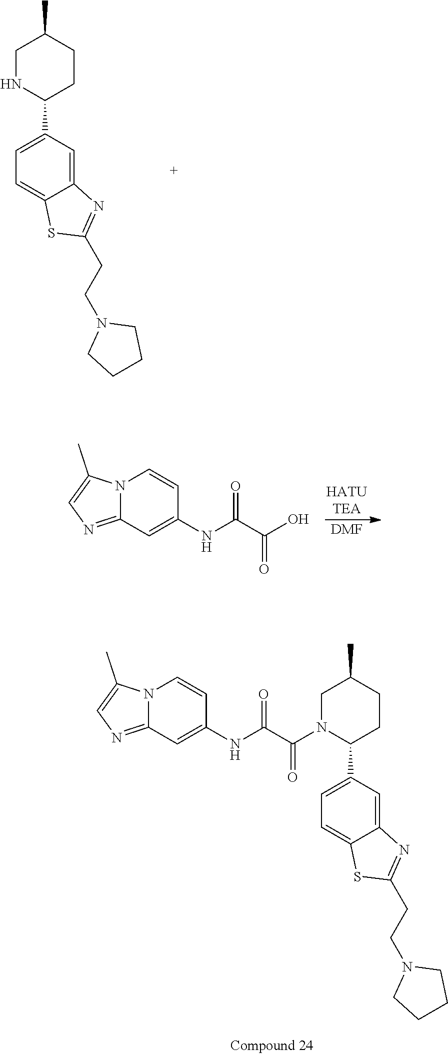

The synthesis of 5-((2R,5S)-5-methylpiperidin-2-yl)-2-(2-(pyrrolidin-1-yl)ethyl)benzo[d]thiazole is given in Intermediate 23.

Prepared by general procedure scheme S2 step 6A. Yield: 26.6 mg (14.68%) using Intermediate 34.

HPLC conditions: Column: XBridge 100*19 mm, 5 microM; 0-1-6 min 40-40-65% water-MeCN+0.1% NH₄OH, flow: 30 ml/min; (loading pump 4 ml/min MeOH).

Compound 24: ¹H NMR (600 MHz, DMSO-d₆) δ (ppm) 1.02-1.08 (m, 3H), 1.31-1.42 (m, 1H), 1.65-1.75 (m, 5H), 1.81-1.95 (m, 1H), 2.06-2.23 (m, 1H), 2.24-2.36 (m, 1H), 2.37-2.43 (m, 3H), 2.50-2.54 (m, 4H), 2.79-3.12 (m, 3H), 3.22-3.26 (m, 2H), 3.49-4.03 (m, 1H), 5.27-5.75 (m, 1H), 7.05-7.21 (m, 1H), 7.23-7.31 (m, 1H), 7.33-7.42 (m, 1H), 7.84-7.88 (m, 1H), 7.88-7.99 (m, 1H), 8.00-8.08 (m, 1H), 8.13-8.26 (m, 1H), 10.95-11.20 (m, 1H). LCMS(ESI): [M]⁺ m/z: calcd 530.2; found 531.2; Rt=1.654 min.

### Example 10. Synthesis of 2-((2R,5S)-5-methyl-2-(2-(1-methylpiperidin-4-yl)benzo[d]thiazol-5-yl)piperidin-1-yl)-2-oxo-N-(5-oxo-5,6,7,8-tetrahydro-1,6-naphthyridin-3-yl)acetamide (Compound 55)

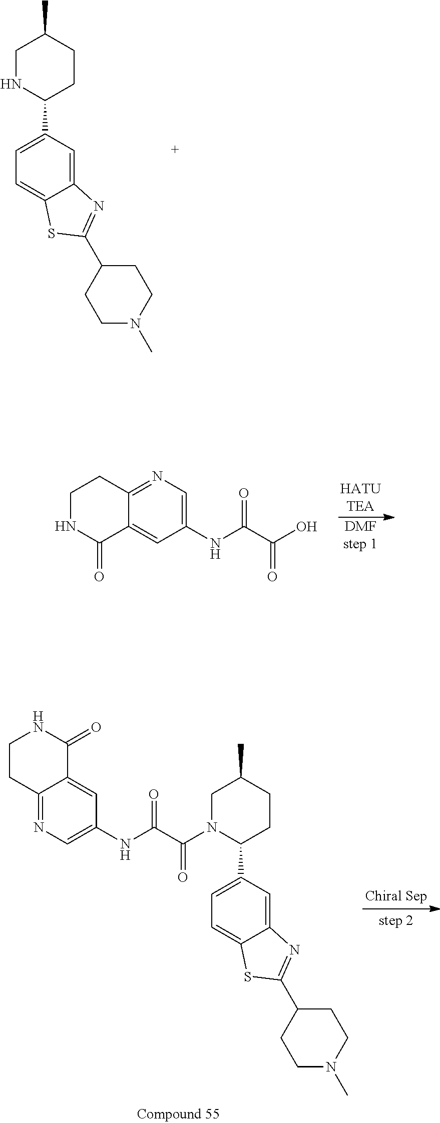 [SMILES: C[C@H]1CC[C@H](c2ccc3sc(C4CCN(C)CC4)nc3c2)NC1 ; O=C(O)C(=O)Nc1cnc2c(c1)C(=O)NCC2 ; "DMFstep 1" ; C[C@H]1CC[C@H](c2ccc3sc(C4CCN(C)CC4)nc3c2)N(C(=O)C(=O)Nc2cnc3c(c2)C(=O)NCC3)C1]

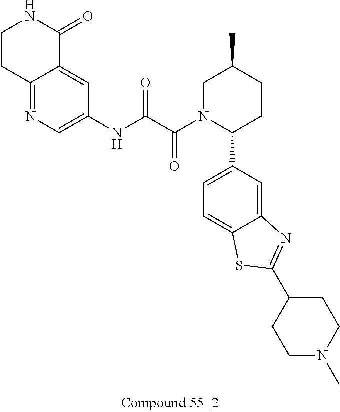 [SMILES: C[C@H]1CC[C@H](c2ccc3sc(C4CCN(C)CC4)nc3c2)N(C(=O)C(=O)Nc2cnc3c(c2)C(=O)NCC3)C1]

The synthesis of 5-((2R,5S)-5-methylpiperidin-2-yl)-2-(1-methylpiperidin-4-yl)benzo[d]thiazole is given in Intermediate 2.

### Step 1: Synthesis of 2-((2R,5S)-5-methyl-2-(2-(1-methylpiperidin-4-yl)benzo[d]thiazol-5-yl)piperidin-1-yl)-2-oxo-N-(5-oxo-5,6,7,8-tetrahydro-1,6-naphthyridin-3-yl)acetamide

Prepared by general procedure scheme S2 step 6A. Yield: 100 mg (54.79%) using Intermediate 36.

HPLC conditions: Column: YMC Triart C18 100*20 mm, 5 microM; 0-1-5 min 45-45-100% water-MeOH+0.1% NH₄OH; (loading pump 4 ml/min MeOH).

Compound 55: ¹H NMR (600 MHz, DMSO-d₆) δ (ppm) 0.99-1.07 (m, 3H), 1.31-1.40 (m, 1H), 1.69-1.94 (m, 4H), 1.98-2.15 (m, 5H), 2.18 (s, 3H), 2.31 (d, 1H), 2.83 (d, 2H), 2.94-3.02 (m, 2H), 3.03-3.08 (m, 1H), 3.31-3.46 (m, 3H), 3.48-4.05 (m, 1H), 5.26-5.73 (m, 1H), 7.34-7.44 (m, 1H), 7.87-7.91 (m, 1H), 8.01-8.09 (m, 1H), 8.09-8.17 (m, 1H), 8.38-8.52 (m, 1H), 8.74-8.85 (m, 1H), 11.10-11.34 (m, 1H). LCMS(ESI): [M]⁺ m/z: calcd 546.2; found 547.2; Rt=2.591 min.

### Step 2: Chiral Separation (Compound 55)

2-[(2R,5S)-5-Methyl-2-[2-(1-methyl-4-piperidyl)-1,3-benzothiazol-5-yl]-1-piperidyl]-2-oxo-N-(5-oxo-7,8-dihydro-6H-1,6-naphthyridin-3-yl)acetamide (90 mg, 164.63 μmol) was purified by chiral HPLC: (Column: Chiralcel OD-H (250*30 mm, 5 mkm), Hexane-IPA-MeOH, 50-25-25. Flow Rate: 25 mL/min) to obtain 2-[(2R,5S)-5-methyl-2-[2-(1-methyl-4-piperidyl)-1,3-benzothiazol-5-yl]-1-piperidyl]-2-oxo-N-(5-oxo-7,8-dihydro-6H-1,6-naphthyridin-3-yl)acetamide (72 mg, 131.70 μmol, 80.00% yield). Rel Time for Compound 55_2 in analytical conditions (column: OD-H, Hexane-IPA-MeOH, 50-25-25, 0.6 ml/min as mobile phase) 21.44 min.

Compound 55_2: Retention time: 21.44 min. ¹H NMR (600 MHZ, DMSO-d₆) δ (ppm) 1.02-1.05 (m, 3H), 1.32-1.40 (m, 2H), 1.70-1.91 (m, 4H), 2.07 (m, 4H), 2.21 (s, 3H), 2.30-2.37 (m, 1H), 2.86 (m, 2H), 2.94-3.07 (m, 3H), 3.39-4.04 (m, 4H), 5.28-5.71 (m, 1H), 7.36-7.42 (m, 1H), 7.88-7.90 (m, 1H), 8.03-8.15 (m, 2H), 8.40-8.51 (d, 1H), 8.76-8.84 (d, 1H), 11.17-11.28 (d, 1H). LCMS(ESI): [M]⁺ m/z: calcd 546.2; found 547.2; Rt=2.231 min.

### Example 11. Synthesis of 2-(2-(2-(2-(dimethylamino)ethyl)benzo[d]thiazol-5-yl)-5-methylpiperidin-1-yl)-2-oxo-N-(1H-pyrazolo[4,3-c]pyridin-7-yl)acetamide (Compound 70 and Compound 5)

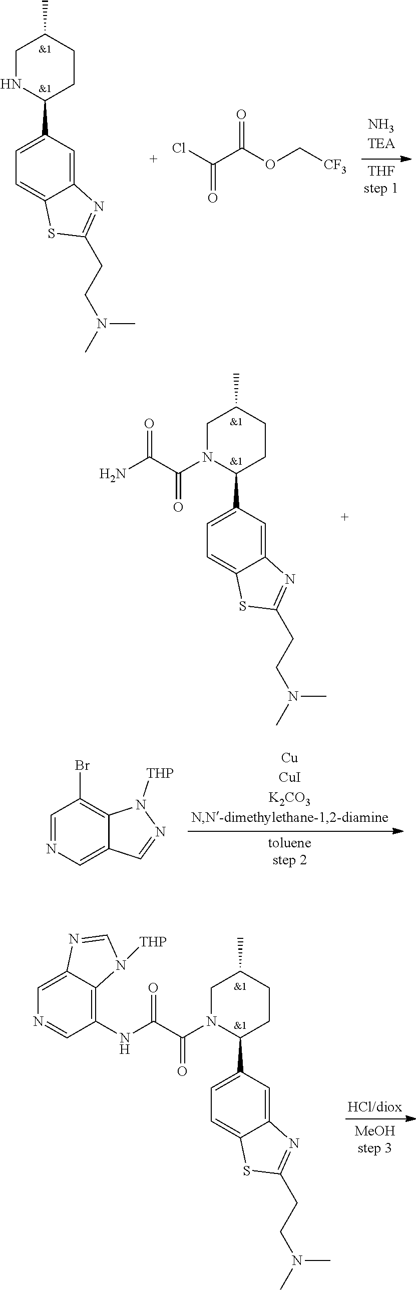

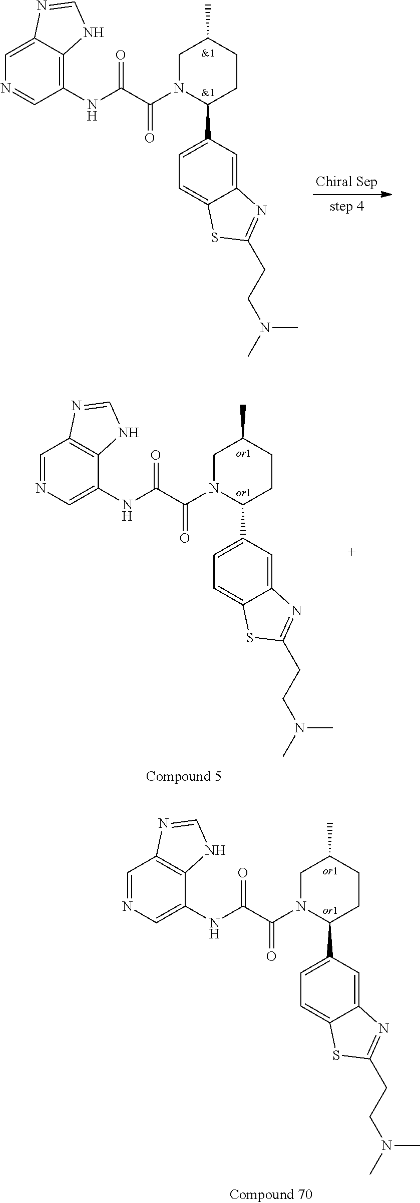 [SMILES: C[C@@H]1CC[C@@H](c2ccc3sc(CCN(C)C)nc3c2)N(C(=O)C(=O)Nc2cncc3nc[nH]c23)C1 ; C[C@H]1CC[C@H](c2ccc3sc(CCN(C)C)nc3c2)N(C(=O)C(=O)Nc2cncc3nc[nH]c23)C1 ; C[C@@H]1CC[C@@H](c2ccc3sc(CCN(C)C)nc3c2)N(C(=O)C(=O)Nc2cncc3nc[nH]c23)C1]

The synthesis of rac-N,N-dimethyl-2-(5-((2R,5S)-5-methylpiperidin-2-yl)benzo[d]thiazol-2-yl)ethanamine is given in Intermediate 24.

### Step 1: Synthesis of rac-2-((2R,5S)-2-(2-(2-(dimethylamino)ethyl)benzo[d]thiazol-5-yl)-5-5 methylpiperidin-1-yl)-2-oxoacetamide

Prepared by general procedure Scheme S5 step 1. Yield: 0.7 mg of crude.

LCMS(ESI): [M]⁺ m/z: calcd 374.2; found 375.2; Rt=0.777 min.

### Step 2: Synthesis of rac-2-((2R,5S)-2-(2-(2-(dimethylamino)ethyl)benzo[d]thiazol-5-yl)-5-methylpiperidin-1-yl)-2-oxo-N-(1-(tetrahydro-2H-pyran-2-yl)-1H-pyrazolo[4,3-c]pyridin-7-yl)acetamide 10

Prepared by general procedure Scheme S5 step 2A. Yield: 90 mg (23.42%).

HPLC conditions: Column: XBridge C18 100*19 mm, 5 microM; 0-1-6 min 25-25-60% water-MeCN+0.1% NH₄OH, flow: 30 ml/min; (loading pump 4 ml/min MeCN).

LCMS(ESI): [M]⁺ m/z: calcd 575.2; found 576.2; Rt=2.357 min.

### Step 3: Synthesis of 2-(2-(2-(2-(dimethylamino)ethyl)benzo[d]thiazol-5-yl)-5-methylpiperidin-1-yl)-2-oxo-N-(1H-pyrazolo[4,3-c]pyridin-7-yl)acetamide

To a solution of 2-[2-[2-[2-(dimethylamino)ethyl]-1,3-benzothiazol-5-yl]-5-methyl-1-piperidyl]-2-oxo-N-(1-tetrahydropyran-2-ylpyrazolo[4,3-c]pyridin-7-yl)acetamide (0.09 g, 156.33 μmol) in MeOH (10 mL) was added hydrogen chloride solution 4.0M in dioxane (800.00 mg, 21.94 mmol, 1 mL) at 21° C. The resulting mixture was left to stir for 5 hr. The resulting mixture was evaporated to dryness and subjected to HPLC (40-40-80% 0-1-6 min H₂O/MeOH/0.1, % NH₄OH flow: 30 ml/min; column: YMC Triart C18 100×20 mm 5 um) to afford 2-[2-[2-[2-(dimethylamino)ethyl]-1,3-benzothiazol-5-yl]-5-methyl-1-piperidyl]-2-oxo-N-(1H-pyrazolo[4,3-c]pyridin-7-yl)acetamide (0.05 g, 101.71 μmol, 65.06% yield). LCMS(ESI): [M]⁺ m/z: calcd 491.2; found 492.2; Rt=1.380 min.

### Step 4: Chiral Separation (Compound 70 and Compound 5)

Racemic 2-[2-[2-[2-(dimethylamino)ethyl]-1,3-benzothiazol-5-yl]-5-methyl-1-piperidyl]-2-oxo-N-(1H-pyrazolo[4,3-c]pyridin-7-yl)acetamide (47.20 mg, 96.01 μmol) was chiral separated (Column: ChiralArt YMC (250-20 mm-5 m); Mobile phase: IPA-MeOH, 50-50 Flow Rate: 10 mL/min) to obtain rel-2-((2R,5S)-2-(2-(2-(dimethylamino)ethyl)benzo[d]thiazol-5-yl)-5-methylpiperidin-1-yl)-2-oxo-N-(1H-pyrazolo[4,3-c]pyridin-7-yl)acetamide (0.05775 g, 124.60 μmol, 50.22% yield) and rel-2-((2R,5S)-2-(2-(2-(dimethylamino)ethyl)benzo[d]thiazol-5-yl)-5-methylpiperidin-1-yl)-2-oxo-N-(1H-pyrazolo[4,3-c]pyridin-7-yl)acetamide (0.01966 g, 39.99 μmol, 41.65% yield).

Rel Time for Compound 70 in analytical conditions (column: IC, IPA-MeOH, 50-50, 0.6 ml/min as mobile phase) 33.69 min and for Compound 5 50.94 min.

Compound 70: Retention time: 33.69 min. ¹H NMR (600 MHz, DMSO-d₆) δ (ppm) 1.01-1.09 (m, 3H), 1.31-1.45 (m, 1H), 1.69-1.82 (m, 1H), 1.86-1.98 (m, 1H), 2.09-2.21 (m, 7H), 2.23-2.35 (m, 1H), 2.67-2.70 (m, 2H), 2.87-3.25 (m, 3H), 3.76-4.13 (m, 1H), 5.55-5.89 (m, 1H), 7.33-7.46 (m, 1H), 7.86-7.91 (m, 1H), 7.97-8.08 (m, 1H), 8.28-8.37 (m, 1H), 8.41-8.55 (m, 1H), 8.87-8.98 (m, 1H), 11.18 (s, 1H), 13.06 (s, 1H). LCMS(ESI): [M]⁺ m/z: calcd 491.2; found 492.2; Rt=0.805 min.

Compound 5: Retention time: 50.94 min. ¹H NMR (600 MHZ, DMSO-d₆) δ (ppm) 1.01-1.09 (m, 3H), 1.31-1.45 (m, 1H), 1.69-1.82 (m, 1H), 1.86-1.98 (m, 1H), 2.09-2.21 (m, 7H), 2.23-2.35 (m, 1H), 2.67-2.70 (m, 2H), 2.87-3.25 (m, 3H), 3.76-4.13 (m, 1H), 5.55-5.89 (m, 1H), 7.33-7.46 (m, 1H), 7.86-7.91 (m, 1H), 7.97-8.08 (m, 1H), 8.28-8.37 (m, 1H), 8.41-8.55 (m, 1H), 8.87-8.98 (m, 1H), 11.18 (s, 1H), 13.06 (s, 1H). LCMS(ESI): [M]⁺ m/z: calcd 491.2; found 492.2; Rt=0.807 min.

### Example 12. Alternative Synthesis of 2-((2R,5S)-2-(2-(2-(dimethylamino)ethyl)benzo[d]thiazol-5-yl)-5-methylpiperidin-1-yl)-2-oxo-N-(1H-pyrazolo[4,3-c]pyridin-7-yl)acetamide (Compound 5)

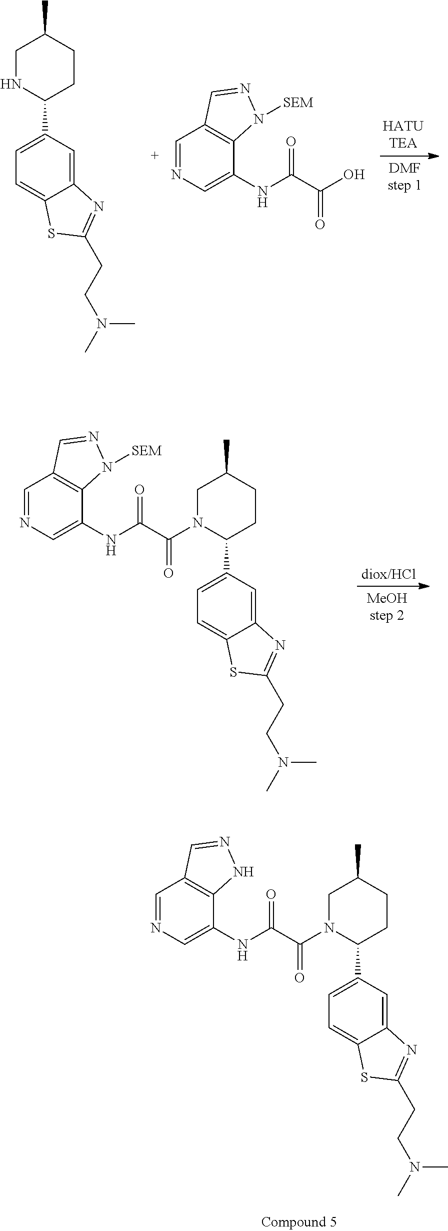 [SMILES: C[C@H]1CC[C@H](c2ccc3sc(CCN(C)C)nc3c2)NC1 ; C[Si](C)(C)CCOCn1ncc2cncc(NC(=O)C(=O)O)c21 ; C[C@H]1CC[C@H](c2ccc3sc(CCN(C)C)nc3c2)N(C(=O)C(=O)Nc2cncc3cnn(COCC[Si](C)(C)C)c23)C1 ; C[C@H]1CC[C@H](c2ccc3sc(CCN(C)C)nc3c2)N(C(=O)C(=O)Nc2cncc3cn[nH]c23)C1]

The synthesis of N,N-dimethyl-2-[5-[(2R,5S)-5-methyl-2-piperidyl]-1,3-benzothiazol-2-yl]ethanamine is given in Intermediate 1.

### Step 1: Synthesis of 2-((2R,5S)-2-(2-(2-(dimethylamino)ethyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

HATU (576.36 mg, 1.52 mmol) was added portion wise at rt to a suspension of 2-oxo-2-[[1-(2-trimethylsilylethoxymethyl)pyrazolo[4,3-c]pyridin-7-yl]amino]acetic acid (Intermediate 37)(487.78 mg, 1.45 mmol), N,N-dimethyl-2-[5-[(2R,5S)-5-methyl-2-piperidyl]-1,3-benzothiazol-2-yl]ethanamine (400 mg, 1.32 mmol) and TEA (800.28 mg, 7.91 mmol, 1.10 mL) in DMF (10.05 mL). The clear solution was stirred at 20° C. for 12 hr and the solvents were evaporated in vacuum. The residue was purified by RP-HPLC (column: XBridge C18 100×19 mm, 5 um; 35-70% 0-5 min H₂O/MeCN/0.1% NH₄OH, flow: 30 ml/min) to give 2-oxo-2-[(2R,5S)-2-[2-[2-(dimethylamino)ethyl]-1,3-benzothiazol-5-yl]-5-methyl-1-piperidyl]-N-[1-(2-trimethylsilyl ethoxymethyl)pyrazolo[4,3-c]pyridin-7-yl]acetamide (460 mg, 739.71 μmol, 56.12% yield). LCMS(ESI): [M]⁺ m/z: calcd 621.2; found 622.2; Rt=3.367 min.

### Step 2: Synthesis of N-(6-amino-5-ethylpyridin-3-yl)-2-((2R,5S)-2-(2-(2-(dimethylamino)ethyl)benzo[d]thiazol-5-yl)-5-methylpiperidin-1-yl)-2-oxoacetamide (Compound 5)

To a solution of 2-oxo-2-[(2R,5S)-2-[2-[2-(dimethylamino)ethyl]-1,3-benzothiazol-5-yl]-5-methyl-1-piperidyl]-N-[1-(2-trimethylsilylethoxymethyl)pyrazolo[4,3-c]pyridin-7-yl]acetamide (460 mg, 739.71 μmol) in methanol (3 mL) was added hydrogen chloride solution 4.0 M in dioxane (2.70 g, 7.40 mmol, 3.37 mL, 10% purity) at 21° C. The resulting mixture was left to stir for 3 day. The resulting mixture was evaporated to dryness. The residue was purified by RP-HPLC (column: XBridge C18 100×19 mm, 5 um; 40-90% 0-5 min H₂O/MeOH/0.1% NH₄OH, flow: 30 ml/min) then by chiral HPLC (X-Bridge C18 (100*19, 5 mkm), H₂O-MeCN—NH₄HCO₃ (0.1%), 45-65, 30 ml/min) to give Compound 5 2-oxo-N-(1H-pyrazolo[4,3-c]pyridin-7-yl)-2-[(2R,5S)-2-[2-[2-(dimethylamino)ethyl]-1,3-benzothiazol-5-yl]-5-methyl-1-piperidyl]acetamide (73 mg, 148.49 μmol, 20.07% yield).

Compound 5: ¹H NMR (600 MHz, dmso) δ 1.02-1.11 (m, 3H), 1.32-1.45 (m, 1H), 1.68-1.82 (m, 1H), 1.86-1.98 (m, 1H), 2.06-2.19 (m, 1H), 2.20 (s, 6H), 2.28-2.36 (m, 1H), 2.65-2.74 (m, 2H), 2.85-3.26 (m, 3H), 3.76-4.19 (m, 1H), 5.56-5.85 (m, 1H), 7.29-7.44 (m, 1H), 7.84-7.96 (m, 1H), 7.98-8.13 (m, 1H), 8.26-8.41 (m, 1H), 8.41-8.55 (m, 1H), 8.86-9.03 (m, 1H), 10.85-11.37 (m, 1H), 12.76-13.43 (m, 1H). LCMS(ESI): [M]⁺ m/z: calcd 491.2; found 492.2; Rt=2.754 min.

### Example 13. Synthesis of 2-((2R,5S)-5-methyl-2-(2-(4-methylpiperazin-1-yl)benzo[d]thiazol-5-yl)piperidin-1-yl)-2-oxo-N-(1H-pyrazolo[4,3-c]pyridin-7-yl)acetamide (Compound 40)

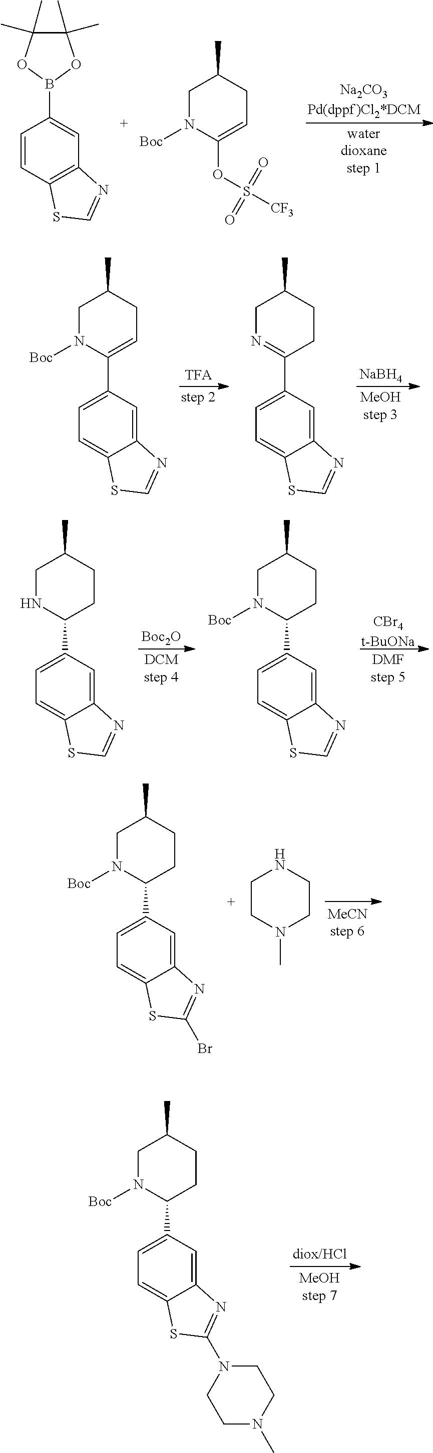

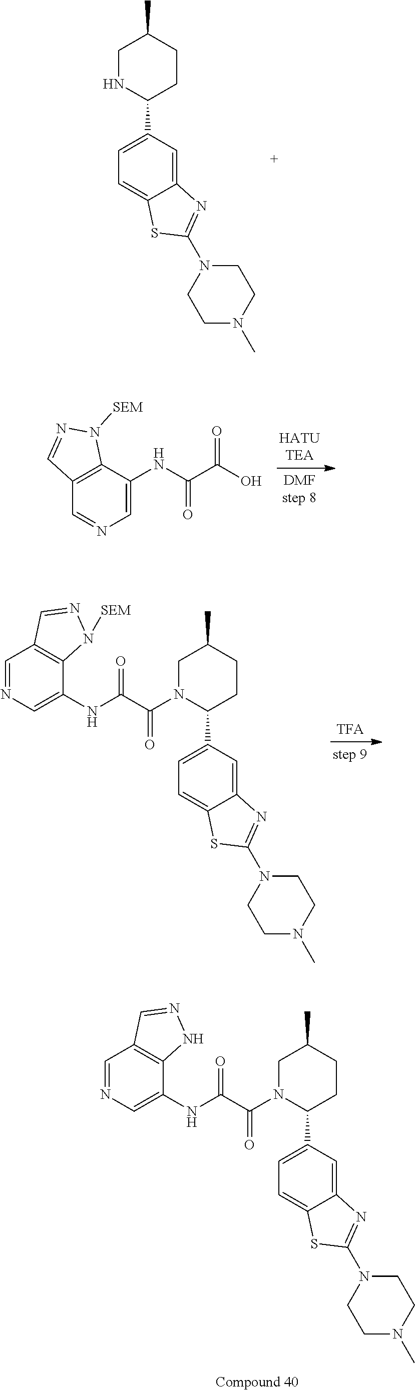 [SMILES: C[C@H]1CC[C@H](c2ccc3sc(N4CCN(C)CC4)nc3c2)NC1 ; C[Si](C)(C)CCOCn1ncc2cncc(NC(=O)C(=O)O)c21 ; C[C@H]1CC[C@H](c2ccc3sc(N4CCN(C)CC4)nc3c2)N(C(=O)C(=O)Nc2cncc3cnn(COCC[Si](C)(C)C)c23)C1 ; C[C@H]1CC[C@H](c2ccc3sc(N4CCN(C)CC4)nc3c2)N(C(=O)C(=O)Nc2cncc3cn[nH]c23)C1]

### Step 1: Synthesis of(S)-tert-butyl 6-(benzo[d]thiazol-5-yl)-3-methyl-3,4-dihydropyridine-1(2H)-carboxylate

Prepared by general procedure scheme S2 step 3. Yield: 3 g (45.56%).

CC conditions: The crude product was purified by silica gel with hexane/MTBE as an eluent mixture.

LCMS(ESI): [M]⁺ m/z: calcd 329.2; found 330.2; Rt=1.635 min.

### Step 2: Synthesis of(S)-5-(5-methyl-3,4,5,6-tetrahydropyridin-2-yl)benzo[d]thiazole

Prepared by general procedure scheme S2 step 4. Yield: 2 g (95.77%).

LCMS(ESI): [M]⁺ m/z: calcd 229.2; found 230.2; Rt=0.783 min.

### Step 3: Synthesis of 5-((2R,5S)-5-methylpiperidin-2-yl)benzo[d]thiazole

Prepared by general procedure scheme S2 step 5. Yield: 2 g (99.13%).

LCMS(ESI): [M]⁺ m/z: calcd 231.2; found 232.2; Rt=0.795 min.

### Step 4: Synthesis of (2R,5S)-tert-butyl 2-(benzo[d]thiazol-5-yl)-5-methylpiperidine-1-carboxylate

Di-tert-butyl dicarbonate (493.15 mg, 2.26 mmol, 518.56 μL) was added in one portion to a stirred solution of 5-[(2R,5S)-5-methyl-2-piperidyl]-1,3-benzothiazole (0.5 g, 2.15 mmol) in DCM (15 mL) at 25° C. The resulting mixture was stirred at 25° C. for 1 hr, and then concentrated in vacuum to afford tert-butyl (2R,5S)-2-(1,3-benzothiazol-5-yl)-5-methyl-piperidine-1-carboxylate (800 mg, crude) as brown gum, which crystallized on standing to give brown solid. LCMS(ESI): [M]⁺ m/z: calcd 332.2; found 333.2; Rt=3.995 min.

### Step 5: Synthesis of (2R,5S)-tert-butyl 2-(2-bromobenzo[d]thiazol-5-yl)-5-methylpiperidine-1-carboxylate

Sodium tert-butoxide (563.68 mg, 5.87 mmol) was added in one portion to a stirred solution of tert-butyl (2R,5S)-2-(1,3-benzothiazol-5-yl)-5-methyl-piperidine-1-carboxylate (650 mg, 1.96 mmol) and carbon tetrabromide (778.05 mg, 2.35 mmol, 227.50 μL) in DMF (10 mL) at 25° C. (slightly exothermic reaction). The resulting mixture was stirred at 25° C. for 12 hr, then diluted with water (15 ml) and extracted with MTBE (3*15 ml). The combined organic extracts were washed with water (2*5 ml), dried over sodium sulphate and concentrated in vacuum to afford tert-butyl (2R,5S)-2-(2-bromo-1,3-benzothiazol-5-yl)-5-methyl-piperidine-1-carboxylate (750 mg, 1.82 mmol, 93.25% yield) as brown solid, which was directly used in the next step. LCMS(ESI): [M]⁺ m/z: calcd 411.2; found 412.2; Rt=4.734 min.

### Step 6: Synthesis of (2R,5S)-tert-butyl 5-methyl-2-(2-(4-methylpiperazin-1-yl)benzo[d]thiazol-5-yl)piperidine-1-carboxylate

1-Methylpiperazine (852.22 mg, 8.51 mmol, 943.77 μL) was added in one portion at 25° C. to a solution of tert-butyl (2R,5S)-2-(2-bromo-1,3-benzothiazol-5-yl)-5-methyl-piperidine-1-carboxylate (700 mg, 1.70 mmol) in MeCN (20 mL). The resulting mixture was stirred at 70° C. for 8 hr, then cooled down and concentrated in vacuum. The residue was basified to pH 11 with 10% aqueous sodium hydroxide solution and extracted with DCM (2*25 ml). The combined organic extracts were dried over sodium sulphate and concentrated in vacuum to afford tert-butyl (2R,5S)-5-methyl-2-[2-(4-methylpiperazin-1-yl)-1,3-benzothiazol-5-yl]piperidine-1-carboxylate (750 mg, crude) as brown solid, which was used directly in the next step. LCMS(ESI): [M]⁺ m/z: calcd 430.2; found 431.2; Rt=3.638 min.

### Step 7: Synthesis of 2-(4-methylpiperazin-1-yl)-5-((2R,5S)-5-methylpiperidin-2-yl)benzo[d]thiazole)

Hydrogen chloride solution 4.0 M in dioxane (31.50 g, 120.09 mmol, 39.38 mL, 13.9% purity) was added in one portion to a stirred solution of tert-butyl (2R,5S)-5-methyl-2-[2-(4-methylpiperazin-1-yl)-1,3-benzothiazol-5-yl]piperidine-1-carboxylate (775.25 mg, 1.80 mmol) in MeOH (25.01 mL). The resulting mixture was stirred at 25° C. for 1 hr, and then evaporated to dryness in vacuum to afford crude 2-(4-methylpiperazin-1-yl)-5-[(2R,5S)-5-methyl-2-piperidyl]-1,3-benzothiazole (900 mg, crude, ³HCl) as brown solid, which was used directly in the next step. LCMS(ESI): [M]⁺ m/z: calcd 330.2; found 331.2; Rt=0.544 min.

### Step 8: Synthesis of 2-((2R,5S)-5-methyl-2-(2-(4-methylpiperazin-1-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A using Intermediate 37. Yield: 15 mg (3.89%).

HPLC conditions: Column: YMC Triart C18 100*19 mm, 5 microM; 0-5 min 60-60-80% water-MeOH+0.1% NH₄OH, flow: 30 ml/min; (loading pump 4 ml/min MeOH).

LCMS(ESI): [M]⁺ m/z: calcd 648.2; found 649.2; Rt=2.771 min.

### Step 9: Synthesis of 2-((2R,5S)-5-methyl-2-(2-(4-methylpiperazin-1-yl)benzo[d]thiazol-5-yl)piperidin-1-yl)-2-oxo-N-(1H-pyrazolo[4,3-c]pyridin-7-yl)acetamide (Compound 40)

A solution of 2-[(2R,5S)-5-methyl-2-[2-(4-methylpiperazin-1-yl)-1,3-benzothiazol-5-yl]-1-piperidyl]-2-oxo-N-[1-(2-trimethylsilylethoxymethyl)pyrazolo[4,3-c]pyridin-7-yl]acetamide (15 mg, 23.12 μmol) in TFA (1 g, 8.77 mmol, 675.68 μL) was stirred at 25° C. for 3 hr. The solvent was evaporated and the residue was purified by HPLC (Device (Mobile Phase, Column): SYSTEM 40-90% 0-5 min H₂O/MeOH/0.1% NH₄OH, flow: 30 ml/min (loading pump 4 ml/min MeOH) target mass 518.65 column: XBridge C18 100×19 mm, 5 um) to give 2-[(2R,5S)-5-methyl-2-[2-(4-methylpiperazin-1-yl)-1,3-benzothiazol-5-yl]-1-piperidyl]-2-oxo-N-(1H-pyrazolo[4,3-c]pyridin-7-yl)acetamide (3 mg, 5.78 μmol, 25.02% yield).

Compound 40: ¹H NMR (600 MHz, DMSO-d₆) δ (ppm) 1.00-1.12 (m, 3H), 1.29-1.43 (m, 1H), 1.68-1.79 (m, 1H), 1.82-1.96 (m, 1H), 2.07-2.34 (m, 6H), 2.40-2.43 (m, 4H), 2.81-3.01 (m, 1H), 3.52-3.57 (m, 4H), 3.73-4.09 (m, 1H), 5.44-5.71 (m, 1H), 7.04-7.13 (m, 1H), 7.39-7.47 (m, 1H), 7.69-7.80 (m, 1H), 8.27-8.36 (m, 1H), 8.40-8.55 (m, 1H), 8.85-8.95 (m, 1H). LCMS(ESI): [M]⁺ m/z: calcd 518.2; found 519.2; Rt=1.604 min.

### Example 14. Synthesis of 2-((2R,5S)-2-(2-(1-((dimethylamino)methyl)cyclopropyl)benzo[d]thiazol-5-yl)-5-methylpiperidin-1-yl)-2-oxo-N-(1H-pyrazolo[4,3-c]pyridin-7-yl)acetamide (Compound 60)

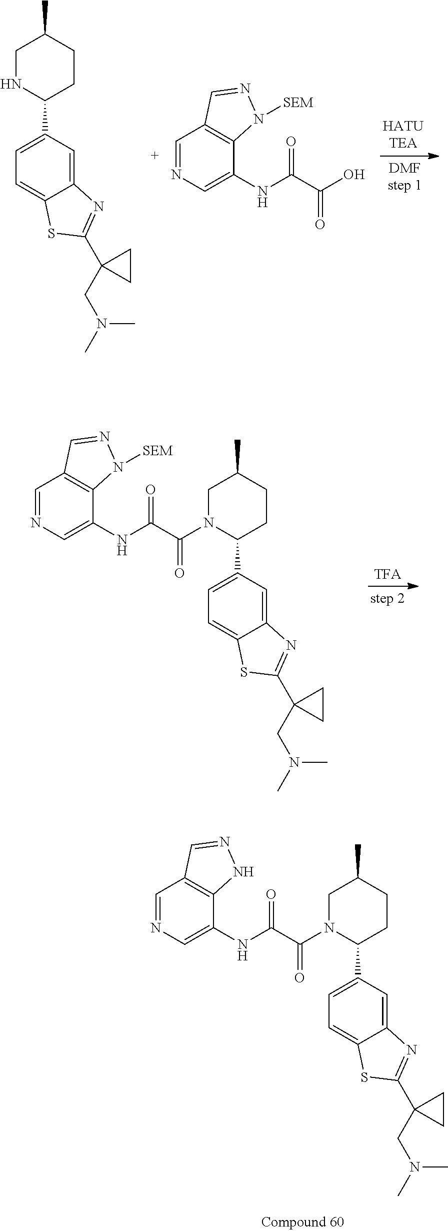 [SMILES: C[C@H]1CC[C@H](c2ccc3sc(C4(CN(C)C)CC4)nc3c2)NC1 ; C[Si](C)(C)CCOCn1ncc2cncc(NC(=O)C(=O)O)c21 ; C[C@H]1CC[C@H](c2ccc3sc(C4(CN(C)C)CC4)nc3c2)N(C(=O)C(=O)Nc2cncc3cnn(COCC[Si](C)(C)C)c23)C1 ; C[C@H]1CC[C@H](c2ccc3sc(C4(CN(C)C)CC4)nc3c2)N(C(=O)C(=O)Nc2cncc3cn[nH]c23)C1]

The Synthesis of N,N-dimethyl-1-(1-(5-((2R,5S)-5-methylpiperidin-2-yl)benzo[d]thiazol-2-yl)cyclopropyl) methanamine is given in Intermediate 5.

### Step 1: Synthesis of 2-((2R,5S)-2-(2-(1-((dimethylamino)methyl)cyclopropyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A using Intermediate 37. Yield: 268 mg (27.26%).

HPLC conditions: Column: XBridge BEH C18 100*20 mm, 5 microM; 0-1-6 min 65-65-90% water-MeOH+0.1% NH₄OH, flow: 30 ml/min; (loading pump 4 ml/min MeOH).

LCMS(ESI): [M]⁺ m/z: calcd 647.2; found 648.2; Rt=3.742 min.

### Step 2: Synthesis of 2-((2R,5S)-2-(2-(1-((dimethylamino)methyl)cyclopropyl)benzo[d]thiazol-5-yl)-5-methylpiperidin-1-yl)-2-oxo-N-(1H-pyrazolo[4,3-c]pyridin-7-yl)acetamide (Compound 60)

A solution of 2-oxo-2-[(2R,5S)-2-[2-[1-[(dimethylamino)methyl]cyclopropyl]-1,3-benzothiazol-5-yl]-5-methyl-1-piperidyl]-N-[1-(2-trimethylsilylethoxymethyl)pyrazolo[4,3-c]pyridin-7-yl]acetamide (268 mg, 413.64 μmol) in TFA (5.92 g, 51.92 mmol, 4 mL) was stirred at 25° C. for 15 hr. The LCMS of the aliquot showed complete SEM-deprotection. The reaction mixture was concentrated in vacuum, the residue was purified by reverse phase HPLC (column: XBridge C18 100×19 mm, 5 um; mobile phase: 40-90% 0-5 min H₂O/MeOH/0.1% NH₄OH, flow rate: 30 ml/min; (loading pump 4 ml/min MeOH)) to afford Compound 60 2-oxo-N-(1H-pyrazolo[4,3-c]pyridin-7-yl)-2-[(2R,5S)-2-[2-[1-[(dimethylamino)methyl]cyclopropyl]-1,3-benzothiazol-5-yl]-5-methyl-1-piperidyl]acetamide (149 mg, 287.84 μmol, 69.59% yield) as light-yellow solid.

Compound 60: ¹H NMR (DMSO-d6, 600 MHz): δ (ppm) 1.05 (m, 4H), 1.41 (m, 4H), 1.74 (m, 1H), 1.91 (m, 1H), 2.12 (m, 1H), 2.30 (m, 7H), 2.75 (m, 3H), 3.94 (m, 1H), 5.66 (m, 1H), 7.35 (m, 1H), 7.80 (m, 1H), 7.99 (m, 1H), 8.33 (m, 1H), 8.47 (m, 1H), 8.93 (m, 1H), 11.12 (m, 1H), 13.03 (m, 1H). LCMS(ESI): [M]⁺ m/z: calcd 517.2; found 518.2; Rt=1.721 min.

### Example 15. Synthesis of 2-((2R,5S)-5-methyl-2-(2-(1-methylazetidin-3-yl)benzo[d]thiazol-5-yl)piperidin-1-yl)-2-oxo-N-(1H-pyrazolo[4,3-c]pyridin-7-yl)acetamide (Compound 62)

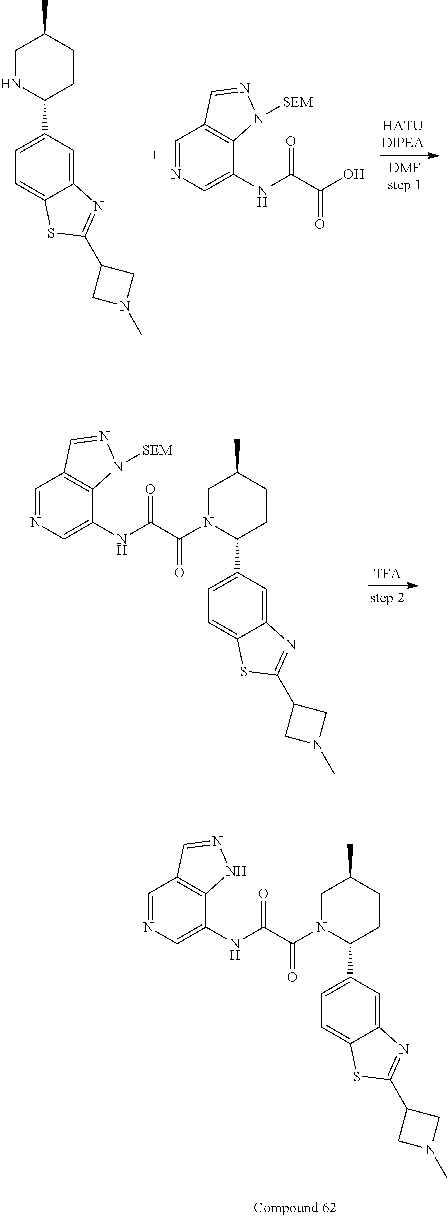 [SMILES: C[C@H]1CC[C@H](c2ccc3sc(C4CN(C)C4)nc3c2)NC1 ; C[Si](C)(C)CCOCn1ncc2cncc(NC(=O)C(=O)O)c21 ; C[C@H]1CC[C@H](c2ccc3sc(C4CN(C)C4)nc3c2)N(C(=O)C(=O)Nc2cncc3cnn(COCC[Si](C)(C)C)c23)C1 ; C[C@H]1CC[C@H](c2ccc3sc(C4CN(C)C4)nc3c2)N(C(=O)C(=O)Nc2cncc3cn[nH]c23)C1]

The synthesis of 2-(1-methylazetidin-3-yl)-5-((2R,5S)-5-methylpiperidin-2-yl)benzo[d]thiazole is given in Intermediate 6.

### Step 1: Synthesis of 2-((2R,5S)-5-methyl-2-(2-(1-methylazetidin-3-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6B using Intermediate 37. Yield: 440 mg (61.14%).

HPLC conditions: Column: XBridge BEH C18 100*20 mm, 5 microM; 0-1-6 min 50-85% water-MeOH+0.1% NH₄OH, flow: 30 ml/min; (loading pump 4 ml/min MeOH).

LCMS(ESI): [M]⁺ m/z: calcd 619.2; found 620.2; Rt=2.979 min.

### Step 2: Synthesis of 2-((2R,5S)-5-methyl-2-(2-(1-methylazetidin-3-yl)benzo[d]thiazol-5-yl)piperidin-1-yl)-2-oxo-N-(1H-pyrazolo[4,3-c]pyridin-7-yl)acetamide (Compound 62)

2-Oxo-2-[(2R,5S)-5-methyl-2-[2-(1-methylazetidin-3-yl)-1,3-benzothiazol-5-yl]-1-piperidyl]-N-[1-(2-trimethylsilylethoxymethyl)pyrazolo[4,3-c]pyridin-7-yl]acetamide (0.46 g, 742.11 μmol) was dissolved in TFA (2.54 g, 22.26 mmol, 1.72 mL) and stirred at 25° C. for 8 hr. Reaction mixture was concentrated, dissolved in MeOH (1 mL), quenched with TEA (0.5 mL), volatiles evaporated and residue submitted to HPLC (column: XBridge BEH C18 5 um 130 A; mobile phase: 20-20-70% 0-1-6 min H₂O/MeOH/0.1% NH₄OH, flow rate: 30 ml/min) to give 2-oxo-N-(1H-pyrazolo[4,3-c]pyridin-7-yl)-2-[(2R,5S)-5-methyl-2-[2-(1-methylazetidin-3-yl)-1,3-benzothiazol-5-yl]-1-piperidyl]acetamide (0.325 g, 663.82 μmol, 89.45% yield).

Compound 62: ¹H NMR (DMSO-d6, 600 MHz): δ (ppm) 1.09 (m, 3H), 1.40 (m, 1H), 1.72 (m, 1H), 1.93 (m, 1H), 2.25 (m, 3H), 2.80 (m, 4H), 3.97 (m, 3H), 4.40 (m, 3H), 5.69 (m, 1H), 7.49 (m, 1H), 7.97 (m, 1H), 8.13 (m, 1H), 8.37 (m, 1H), 8.55 (m, 1H), 8.94 (m, 1H), 11.18 (m, 1H), 13.10 (s, 1H). LCMS(ESI): [M]⁺ m/z: calcd 489.2; found 490.2; Rt=1.950 min.

### Example 16. Synthesis of N-(imidazo[1,2-a]pyridin-7-yl)-2-((2R,5S)-5-methyl-2-(2-(1,2,2,6,6-pentamethylpiperidin-4-yl)benzo[d]thiazol-5-yl)piperidin-1-yl)-2-oxoacetamide (Compound 67)

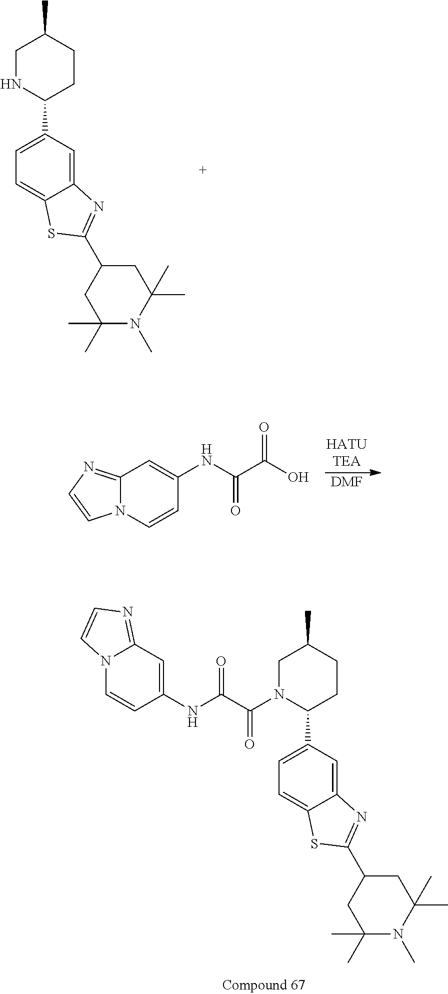

The synthesis of 5-((2R,5S)-5-methylpiperidin-2-yl)-2-(1,2,2,6,6-pentamethylpiperidin-4-yl)benzo[d]thiazole is given in Intermediate 7.

Prepared by general procedure scheme S2 step 6A. Yield: 64.3 mg (28.86%).

HPLC conditions: Column: YMC Triart C18 100*20 mm, 5 microM; 0-1-6 min 60-60-80% water-MeOH+0.1% NH₄OH; (loading pump 4 ml/min MeOH).

Compound 67: ¹H NMR (600 MHZ, DMSO-d₆) δ (ppm) 1.02-1.06 (m, 3H), 1.06-1.09 (m, 6H), 1.11-1.14 (m, 6H), 1.30-1.43 (m, 1H), 1.58-1.67 (m, 2H), 1.68-1.77 (m, 1H), 1.84-1.99 (m, 3H), 2.05-2.19 (m, 1H), 2.20-2.23 (m, 3H), 2.28-2.36 (m, 1H), 2.87-2.91 (m, 0.3H), 3.32-3.34 (m, 0.7H), 3.45-4.05 (m, 2H), 5.24-5.77 (m, 1H), 6.98-7.11 (m, 1H), 7.33-7.43 (m, 1H), 7.43-7.53 (m, 1H), 7.78-7.89 (m, 2H), 7.89-8.02 (m, 1H), 8.02-8.10 (m, 1H), 8.38-8.51 (m, 1H), 10.94 (br s, 1H). LCMS(ESI): [M]⁺ m/z: calcd 572.2; found 573.2; Rt=2.037 min.

### Example 17. Synthesis of 2-((2R,5S)-2-(2-(3-((dimethylamino)methyl) oxetan-3-yl)benzo[d]thiazol-5-yl)-5-methylpiperidin-1-yl)-2-oxo-N-(1H-pyrazolo[4,3-c]pyridin-7-yl)acetamide (Compound 112)

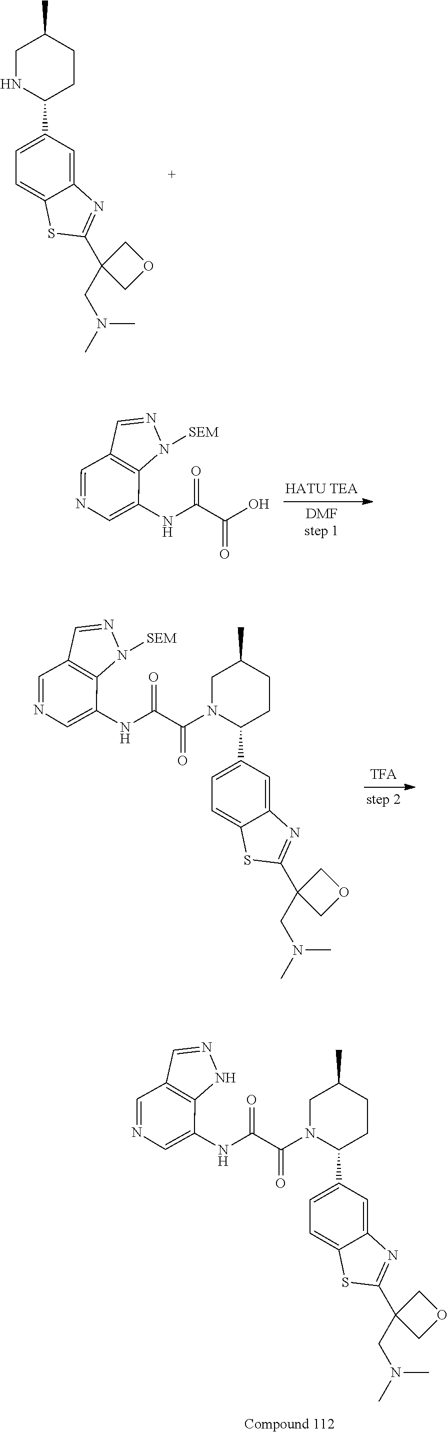 [SMILES: C[C@H]1CC[C@H](c2ccc3sc(C4(CN(C)C)COC4)nc3c2)NC1 ; C[Si](C)(C)CCOCn1ncc2cncc(NC(=O)C(=O)O)c21 ; C[C@H]1CC[C@H](c2ccc3sc(C4(CN(C)C)COC4)nc3c2)N(C(=O)C(=O)Nc2cncc3cnn(COCC[Si](C)(C)C)c23)C1 ; C[C@H]1CC[C@H](c2ccc3sc(C4(CN(C)C)COC4)nc3c2)N(C(=O)C(=O)Nc2cncc3cn[nH]c23)C1]

The synthesis of N,N-dimethyl-1-(3-(5-((2R,5S)-5-methylpiperidin-2-yl)benzo[d]thiazol-2-yl) oxetan-3-yl) methanamine is given in Intermediate 4.

### Step 1: Synthesis of 2-((2R,5S)-2-(2-(3-((dimethylamino)methyl) oxetan-3-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A using Intermediate 37. Yield: 172 mg (35.8%).

HPLC conditions: Column: Chromatorex C18 100*19 mm, 5 microM; 0-1-6 min 60-60-100% water-MeOH, flow: 30 ml/min; (loading pump 4 ml/min MeOH).

LCMS(ESI): [M]⁺ m/z: calcd 663.2; found 664.2; Rt=2.965 min.

### Step 2: Synthesis of 2-((2R,5S)-2-(2-(3-((dimethylamino)methyl) oxetan-3-yl)benzo[d]thiazol-5-yl)-5-methylpiperidin-1-yl)-2-oxo-N-(1H-pyrazolo[4,3-c]pyridin-7-yl)acetamide (Compound 112)

A solution of 2-oxo-2-[(2R,5S)-2-[2-[3-[(dimethylamino)methyl]oxetan-3-yl]-1,3-benzothiazol-5-yl]-5-methyl-1-piperidyl]-N-[1-(2-trimethylsilylethoxymethyl)pyrazolo[4,3-c]pyridin-7-yl]acetamide (155 mg, 233.47 μmol) in TFA (4.44 g, 38.94 mmol, 3 mL) was stirred at 25° C. for 15 hr, and then concentrated in vacuum. The LCMS showed complete SEM-deprotection. The reaction mixture was concentrated in vacuum and the residue was purified by reverse phase HPLC (column: YMC Triart C18 100×20 mm, 5 um; mobile phase: 10-10-50% 0-1-5 min H₂O/MeCN/0.1% NH₄OH, flow rate: 30 ml/min (loading pump 4 ml/min MeCN)) to afford 67 mg of crude product 85% purity by LCMS, which was re-purified by preparative chiral HPLC (column: Chiralpak IJ-I (250*20, 5 mm); mobile phase: Hexane-IPA-MeOH, 50-25-25; flow rate: 12 ml/min) to afford Compound 112 2-oxo-N-(1H-pyrazolo[4,3-c]pyridin-7-yl)-2-[(2R,5S)-2-[2-[3-[(dimethylamino)methyl]oxetan-3-yl]-1,3-benzothiazol-5-yl]-5-methyl-1-piperidyl]acetamide (62 mg, 116.18 μmol, 49.76% yield) as white solid (RetTime=13.265 min.).

Compound 112: ¹H NMR (DMSO-d6, 600 MHz): δ (ppm) 1.07 (m, 3H), 1.39 (m, 1H), 1.77 (m, 1H), 1.93 (m, 1H), 2.09 (s, 6H), 2.15 (m, 1H), 2.30 (m, 1H), 3.07 (m, 2H), 3.39 (m, 1H), 4.02 (m, 1H), 4.75 (m, 2H), 4.95 (m, 2H), 5.70 (m, 1H), 7.44 (m, 1H), 8.05 (m, 2H), 8.33 (m, 1H), 8.48 (m, 1H), 8.93 (m, 1H), 11.14 (m, 1H), 13.04 (m, 1H). LCMS(ESI): [M]⁺ m/z: calcd 533.2;

### Example 18. Synthesis of 2-((2R,5S)-2-(2-(4-hydroxy-1-methylpiperidin-4-yl)benzo[d]thiazol-5-yl)-5-methylpiperidin-1-yl)-2-oxo-N-(1H-pyrazolo[4,3-c]pyridin-7-yl)acetamide (Compound 19)

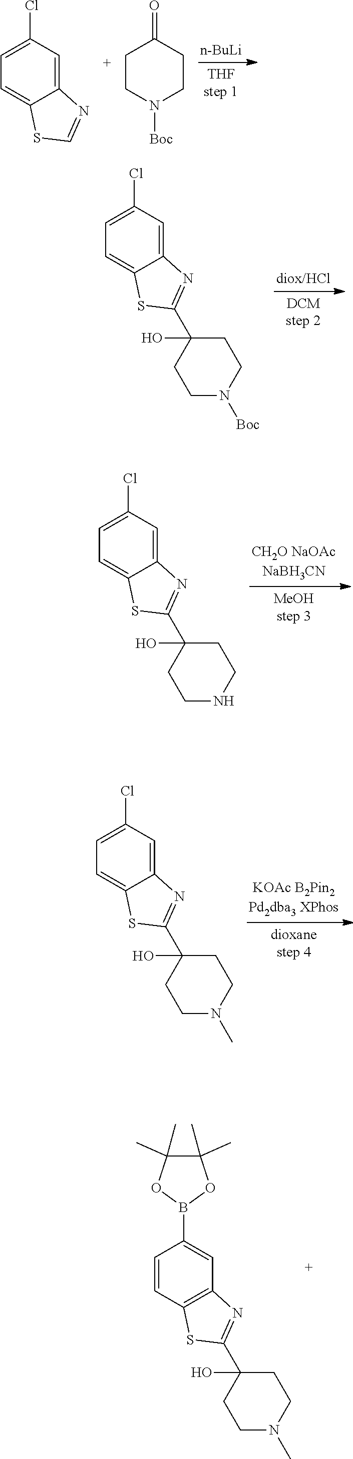

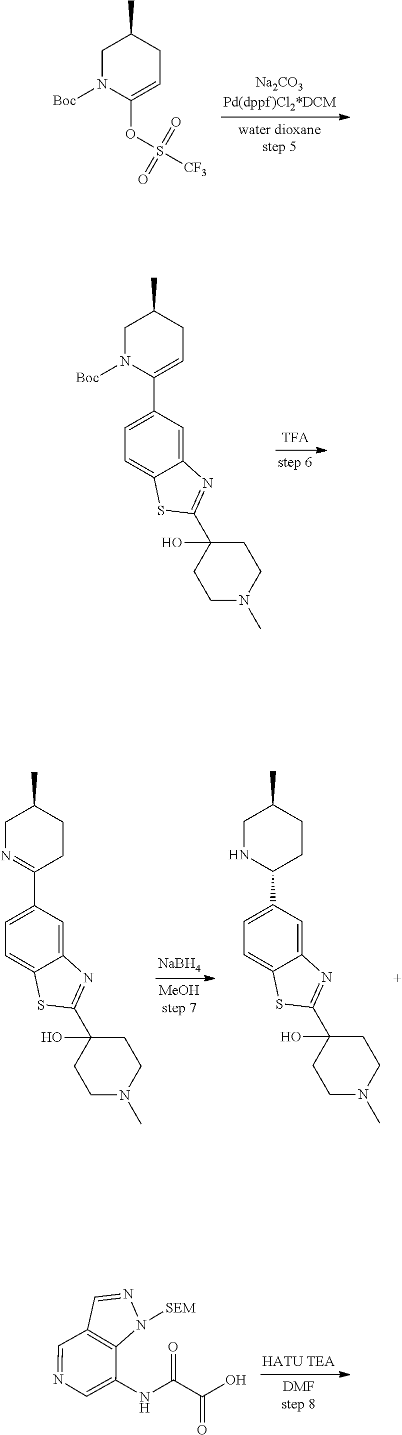 [SMILES: C[C@H]1CC=C(OS(=O)(=O)C(F)(F)F)N(C(=O)OC(C)(C)C)C1 ; C[C@H]1CC=C(c2ccc3sc(C4(O)CCN(C)CC4)nc3c2)N(C(=O)OC(C)(C)C)C1 ; C[C@H]1CCC(c2ccc3sc(C4(O)CCN(C)CC4)nc3c2)=NC1 ; "NaBH4" ; C[C@H]1CC[C@H](c2ccc3sc(C4(O)CCN(C)CC4)nc3c2)NC1 ; C[Si](C)(C)CCOCn1ncc2cncc(NC(=O)C(=O)O)c21]

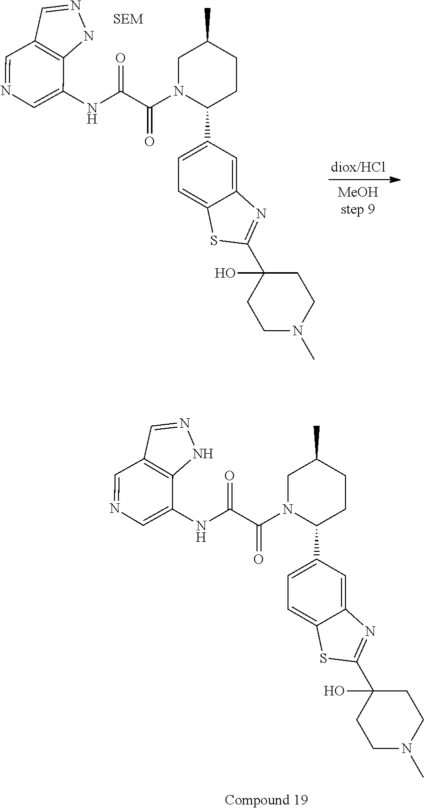 [SMILES: C[C@H]1CC[C@H](c2ccc3sc(C4(O)CCN(C)CC4)nc3c2)N(C(=O)C(=O)Nc2cncc3cn[nH]c23)C1 ; C[C@H]1CC[C@H](c2ccc3sc(C4(O)CCN(C)CC4)nc3c2)N(C(=O)C(=O)Nc2cncc3cn[nH]c23)C1 ; "SEM"]

### Step 1: Synthesis of tert-butyl 4-(5-chlorobenzo[d]thiazol-2-yl)-4-hydroxypiperidine-1-carboxylate

A solution of 5-chloro-1,3-benzothiazole (10 g, 58.95 mmol) in THF (50 mL) was 5 added dropwise to a solution of n-butyllithium solution (18.06 g, 64.85 mmol, 26.06 mL, 23% purity) in THF (50 mL) at −78° C. under argon. After 25 min, a solution of 5-chloro-1,3-benzothiazole (10 g, 58.95 mmol) in THF (50 mL) was added dropwise and the resulting suspension was stirred at −78° C. for 3 hr. The reaction mixture was slowly heated to 25° C. overnight. The reaction mixture was quenched with sat. NH₄Cl. Water (50 mL) was added and product was extracted with MTBE (3*20 ml). The combined organic layers were dried over Na₂SO₄ and the solvents were removed under reduced pressure to give tert-butyl 4-(5-chloro-1,3-benzothiazol-2-yl)-4-hydroxy-piperidine-1-carboxylate (16 g, 43.37 mmol, 73.58% yield). LCMS(ESI): [M]⁺ m/z: calcd 368.2; found 369.2; Rt=1.373 min.

### Step 2: Synthesis of 4-(5-chlorobenzo[d]thiazol-2-yl)piperidin-4-ol

To the stirred solution of tert-butyl 4-(5-chloro-1,3-benzothiazol-2-yl)-4-hydroxy-piperidine-1-carboxylate (3.1 g, 8.40 mmol) in DCM (39.62 mL) hydrogen chloride solution 4.0M in dioxane (306.41 mg, 8.40 mmol, 383.02 μL) was added. The resulting mixture was stirred at 25° C. for 14 hr. Solvents were evaporated in vacuum. The residue was diluted with MTBE (50 ml). Solid was filtered, washed with MTBE, then dried in vacuum to give 4-(5-chloro-1,3-benzothiazol-2-yl)piperidin-4-ol (2.5 g, 8.19 mmol, 97.46% yield, HCl). LCMS(ESI): [M]⁺ m/z: calcd 268.2; found 269.2; Rt=0.799 min.

### Step 3: Synthesis of 4-(5-chlorobenzo[d]thiazol-2-yl)-1-methylpiperidin-4-ol

To the stirred solution of 4-(5-chloro-1,3-benzothiazol-2-yl)piperidin-4-ol (3.1 g, 10.16 mmol, HCl) in MeOH (120 mL), formaldehyde, 37% w/w aq. soln., stab. with 7-8% MeOH (1.24 g, 15.23 mmol, 1.14 mL, 37% purity) and sodium acetate, anhydrous (1.67 g, 20.31 mmol, 1.09 mL) were added. The resulting mixture was stirred for 2 hr at 25° C. Then Sodium cyano borohydride (765.88 mg, 12.19 mmol) was added portion wise. The resulting mixture was stirred at 25° C. for 12 hr. Methanol was evaporated. The residue was diluted with water (50 ml) and extracted with DCM (3*50 ml). Combined organic layers were dried over Na₂SO₄. DCM was evaporated in vacuum to give 4-(5-chloro-1,3-benzothiazol-2-yl)-1-methyl-piperidin-4-ol (2 g, 7.07 mmol, 69.63% yield). LCMS(ESI): [M]⁺ m/z: calcd 282.2; found 283.2; Rt=0.826 min.

### Step 4: Synthesis of 1-methyl-4-(5-(4,4,5,5-tetramethyl-1,3,2-dioxaborolan-2-yl)benzo[d]thiazol-2-yl)piperidin-4-ol

4-(5-Chloro-1,3-benzothiazol-2-yl)-1-methyl-piperidin-4-ol (2 g, 7.07 mmol), 4,4,5,5-tetramethyl-2-(4,4,5,5-tetramethyl-1,3,2-dioxaborolan-2-yl)-1,3,2-dioxaborolane (1.98 g, 7.78 mmol) and potassium acetate (1.39 g, 14.14 mmol, 884.19 μL) were mixed in dioxane (35 mL). The resulting mixture was evacuated and then backfilled with argon, this operation was repeated three times, then tris(dibenzylideneacetone)dipalladium (0)(323.82 mg, 353.62 μmol) and XPhos (337.16 mg, 707.24 μmol) were added under argon. The reaction mixture was stirred under argon at 90° C. for 14 hr. The reaction mixture was cooled down and filtered. The filter cake was washed with dioxane (2*10 ml) and discarded. The combined filtrate was concentrated in vacuum. The residue was diluted with MTBE (50 ml) and extracted with a NaHSO₄ water solution (20 ml) (repeated 3 times). The combined aqueous layer was basified to pH 10 with 10% aqueous sodium hydroxide to give water solution of 1-methyl-4-[5-(4,4,5,5-tetramethyl-1,3,2-dioxaborolan-2-yl)-1,3-benzothiazol-2-yl]piperidin-4-ol (2.6 g, 6.95 mmol, 98.22% yield) which was used directly in the next step. LCMS(ESI): [M]⁺ m/z: calcd 374.2; found 375.2; Rt=1.098 min.

### Step 5: Synthesis of(S)-tert-butyl 6-(2-(4-hydroxy-1-methylpiperidin-4-yl)benzo[d]thiazol-5-yl)-3-methyl-3,4-dihydropyridine-1(2H)-carboxylate

Prepared by general procedure scheme S2 step 3. Yield: 2 g of crude.

LCMS(ESI): [M]⁺ m/z: calcd 443.2; found 444.2; Rt=1.185 min.

### Step 6: Synthesis of(S)-1-methyl-4-(5-(5-methyl-3,4,5,6-tetrahydropyridin-2-yl)benzo[d]thiazol-2-yl)piperidin-4-ol

Prepared by general procedure scheme S2 step 4. Yield: 1.1 g of crude.

LCMS(ESI): [M]⁺ m/z: calcd 343.2; found 344.2; Rt=0.661 min.

### Step 7: Synthesis of 1-methyl-4-(5-((2R,5S)-5-methylpiperidin-2-yl)benzo[d]thiazol-2-yl)piperidin-4-ol

Prepared by general procedure scheme S2 step 5. Yield: 1 g (90.38%).

LCMS(ESI): [M]⁺ m/z: calcd 345.2; found 346.2; Rt=0.677 min.

### Step 8: Synthesis of 2-((2R,5S)-2-(2-(4-hydroxy-1-methylpiperidin-4-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

To the solution of 1-methyl-4-[5-[(2R,5S)-5-methyl-2-piperidyl]-1,3-benzothiazol-2-yl]piperidin-4-ol (0.3 g, 868.30 μmol), 2-oxo-2-[[1-(2-trimethylsilylethoxymethyl)pyrazolo[4,3-c]pyridin-7-yl]amino]acetic acid (Intermediate 37) (292.11 mg, 868.30 μmol) and TEA (263.59 mg, 2.60 mmol, 363.07 μL) in DMF (4 mL) HATU (363.17 mg, 955.13 μmol) was added portion wise. Mixture was stirred at 25° C. for 1.5 hr. The reaction mixture was poured into water (25 ml) and extracted with EtOAc (3*25 ml). Combined organic layers were washed with water (3*25 ml) dried over Na₂SO₄. EtOAc was evaporated in vacuum to give 2-oxo-2-[(2R,5S)-2-[2-(4-hydroxy-1-methyl-4-piperidyl)-1,3-benzothiazol-5-yl]-5-methyl-1-piperidyl]-N-[1-(2-trimethylsilylethoxymethyl)pyrazolo[4,3-c]pyridin-7-yl]acetamide (0.45 g, crude). LCMS(ESI): [M]⁺ m/z: calcd 663.2; found 664.2; Rt=1.108 min.

### Step 9: Synthesis of 2-((2R,5S)-2-(2-(4-hydroxy-1-methylpiperidin-4-yl)benzo[d]thiazol-5-yl)-5-methylpiperidin-1-yl)-2-oxo-N-(1H-pyrazolo[4,3-c]pyridin-7-yl)acetamide (Compound 19)

To the stirred solution of the 2-oxo-2-[(2R,5S)-2-[2-(4-hydroxy-1-methyl-4-piperidyl)-1,3-benzothiazol-5-yl]-5-methyl-1-piperidyl]-N-[1-(2-trimethylsilylethoxymethyl)pyrazolo[4,3-c]pyridin-7-yl]acetamide (0.15 g, 225.94 μmol) in MeOH (2 mL), hydrogen chloride solution 4.0M in dioxane (800.00 mg, 21.94 mmol, 1 mL) was added. The resulting mixture was stirred at 25° C. for 1.5 hr. The reaction mixture was concentrated under reduce pressure. The residue was purified by reverse phase HPLC (SYSTEM 10-10-55% 0-1-6 min H₂O/MeOH/0.1% NH₄OH, flow: 30 ml/min (loading pump 4 ml/min MeOH) target mass 533 column: XBridge BEH C18 5 um 130 A) to give product (31 mg) contains 10% of cis-isomer. Obtained product was submitted for chiral HPLC (Chiralpak IC—III (250*20 mm, 5 mkm), MeOH-IPA, 50-50, 10 ml/min) to give 2-oxo-N-(1H-pyrazolo[4,3-c]pyridin-7-yl)-2-[(2R,5S)-2-[2-(4-hydroxy-1-methyl-4-piperidyl)-1,3-benzothiazol-5-yl]-5-methyl-1-piperidyl]acetamide (23 mg, 43.10 μmol, 19.08% yield).

Compound 19: ¹H NMR (DMSO-d6, 600 MHz): δ (ppm) 1.06 (m, 3H), 1.38 (m, 1H), 1.77 (m, 3H), 1.92 (d, 1H), 2.15 (m, 2H), 2.19 (s, 3H), 2.31 (m, 2H), 2.60 (m, 4H), 2.88 (m, 1H), 3.95 (m, 1H), 5.68 (m, 1H), 6.09 (m, 1H), 7.40 (m, 1H), 7.91 (m, 1H), 8.06 (m, 1H), 8.33 (m, 1H), 8.47 (m, 1H), 8.93 (m, 1H), 11.14 (m, 1H), 13.06 (m, 1H). LCMS(ESI): [M]⁺ m/z: calcd 533.2; found 534.2; Rt=0.663 min.

### Example 19. Synthesis of 2-((2R,5S)-2-(2-(1-(dimethylamino) propan-2-yl)benzo[d]thiazol-5-yl)-5-methylpiperidin-1-yl)-N-(imidazo[1,2-a]pyridin-7-yl)-2-oxoacetamide (Compound 56 and Compound 89)

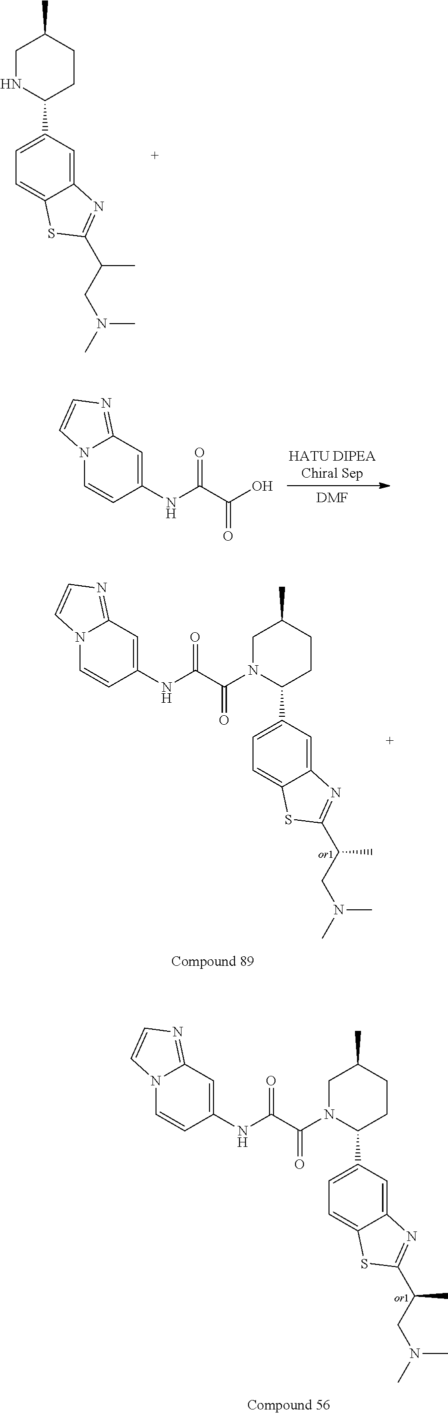

The Synthesis of N,N-dimethyl-2-(5-((2R,5S)-5-methylpiperidin-2-yl)benzo[d]thiazol-2-yl) propan-1-amine is given in Intermediate 8.

To a stirred solution of N,N-dimethyl-2-[5-[(2R,5S)-5-methyl-2-piperidyl]-1,3-benzothiazol-2-yl]propan-1-amine (0.1 g, 314.97 μmol), 2-(imidazo[1,2-a]pyridin-7-ylamino)-2-oxo-acetic acid (0.08 g, 389.92 μmol) and DIPEA (148.40 mg, 1.15 mmol, 0.2 mL) in DMF (8 mL) was added HATU (0.15 g, 394.50 μmol) at 5-10° C. The resulting reaction mixture was stirred at 25° C. for 24 hr. Upon completion, the reaction mixture was concentrated under reduced pressure to obtain crude product. The obtained crude product was purified by reverse phase HPLC chromatography (Device (Mobile Phase, Column): SYSTEM 40-40-85% 0-1-6 min H₂O/MeOH/0.1% NH₄OH, flow: 30 ml/min (loading pump 4 ml/min MeOH) target mass 504 column: YMC Triart C18 100×20 mm, 5 um) to afford crude product (55 mg).

The obtained crude product was purified by chiral column chromatography (Column: Chiralpak IC (250*20 mm, 5 mkm); Mobile phase: Hexan-IPA-MeOH, 50-25-25 Flow Rate: 15 mL/min) to obtain N-imidazo[1,2-a]pyridin-7-yl-2-oxo-2-[(2R,5S)-5-methyl-2-[2-[(¹R)-2-(dimethylamino)-1-methyl-ethyl]-1,3-benzothiazol-5-yl]-1-piperidyl]acetamide (22 mg, 43.59 μmol, 13.84% yield) and N-imidazo[1,2-a]pyridin-7-yl-2-oxo-2-[(2R,5S)-5-methyl-2-[2-[(¹S)-2-(dimethylamino)-1-methyl-ethyl]-1,3-benzothiazol-5-yl]-1-piperidyl]acetamide (22 mg, 43.59 μmol, 13.84% yield).

Rel Time for Compound 56 in analytical conditions (column: IC, Hexane-IPA-MeOH, 50-25-25, 0.6 ml/min as mobile phase) 58.74 min and for Compound 89 48.17 min.

Compound 56: Retention time: 58.74 min. ¹H NMR (600 MHz, dmso) δ 1.03-1.07 (m, 3H), 1.32-1.42 (m, 4H), 1.67-1.78 (m, 1H), 1.84-1.94 (m, 1H), 2.06-2.15 (m, 1H), 2.16-2.24 (m, 6H), 2.30-2.35 (m, 1H), 2.61-2.69 (m, 1H), 2.84-3.15 (m, 1H), 3.34-3.47 (m, 2H), 3.48-4.07 (m, 1H), 5.27-5.77 (m, 1H), 6.97-7.13 (m, 1H), 7.34-7.44 (m, 1H), 7.44-7.53 (m, 1H), 7.80-8.09 (m, 4H), 8.40-8.55 (m, 1H), 11.04-11.23 (m, 1H).

LCMS(ESI): [M]⁺ m/z: calcd 504.2; found 505.2; Rt=0.834 min.

Compound 89: Retention time: 48.17 min. ¹H NMR (600 MHz, dmso) δ 1.02-1.06 (m, 3H), 1.29-1.41 (m, 4H), 1.67-1.77 (m, 1H), 1.84-1.95 (m, 1H), 2.07-2.14 (m, 1H), 2.20 (s, 6H), 2.28-2.34 (m, 1H), 2.85-3.08 (m, 1H), 3.34-3.47 (m, 3H), 3.48-4.07 (m, 1H), 5.23-5.74 (m, 1H), 6.98-7.13 (m, 1H), 7.33-7.44 (m, 1H), 7.45-7.52 (m, 1H), 7.80-8.08 (m, 4H), 8.38-8.54 (m, 1H), 11.04-11.23 (m, 1H). LCMS(ESI): [M]⁺ m/z: calcd 504.2; found 505.2; Rt=0.836 min.

### Example 20. Synthesis of 2-((2R,5S)-5-methyl-2-(2-((1-methylpiperidin-4-yl)methyl)benzo[d]thiazol-5-yl)piperidin-1-yl)-2-oxo-N-(1H-pyrazolo[4,3-c]pyridin-7-yl)acetamide (Compound 61)

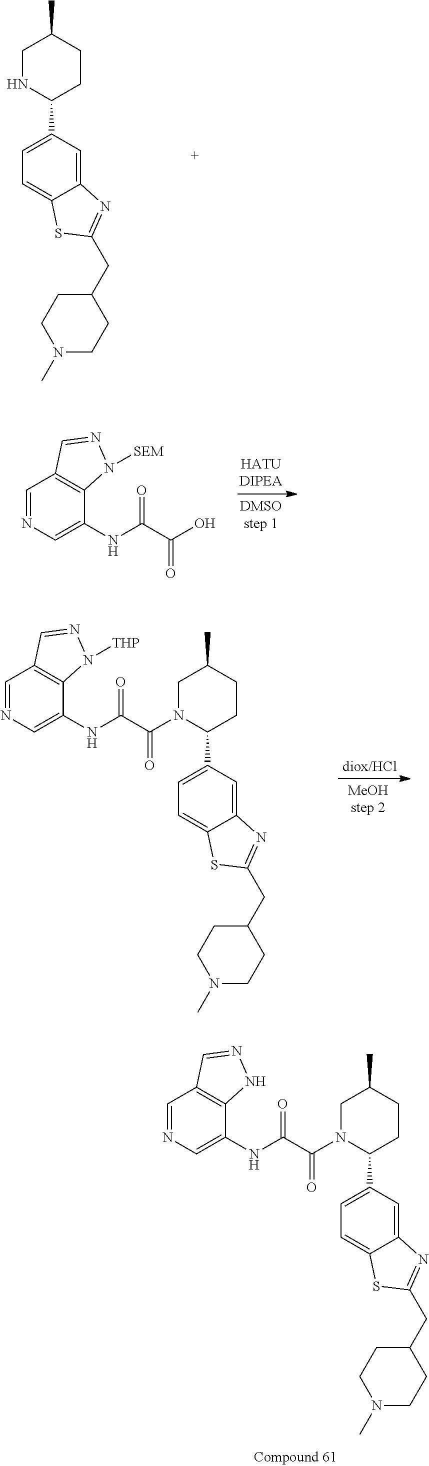

The Synthesis of 2-[(1-methyl-4-piperidyl)methyl]-5-[(2R,5S)-5-methyl-2-piperidyl]-1,3-benzothiazole is given in Intermediate 9.

### Step 1: Synthesis of 2-((2R,5S)-5-methyl-2-(2-((1-methylpiperidin-4-yl)methyl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6B. Yield: 58 mg (30.1%).

HPLC conditions: Column: SunFire 100*19 mm, 5 microM; 2-10 min 10-40% MeOH+FA, flow: 30 ml/min; (loading pump 4 ml/min MeOH+FA).

LCMS(ESI): [M]⁺ m/z: calcd 661.2; found 662.2; Rt=1.290 min.

### Step 2: Synthesis of 2-((2R,5S)-5-methyl-2-(2-((1-methylpiperidin-4-yl)methyl)benzo[d]thiazol-5-yl)piperidin-1-yl)-2-oxo-N-(1H-pyrazolo[4,3-c]pyridin-7-yl)acetamide (Compound 61)

2-Oxo-2-[(2R,5S)-5-methyl-2-[2-[(1-methyl-4-piperidyl)methyl]-2,3-dihydro-1,3-benzothiazol-5-yl]-1-piperidyl]-N-[1-(2-trimethylsilylethoxymethyl)pyrazolo[4,3-c]pyridin-7-yl]acetamide (0.058 g, 87.36 μmol) was dissolved in MeOH (3 mL) and HCl in dioxane (87.36 μmol, 3 mL) was added thereto. Then it was stirred 25° C. for 1 hr. The reaction mixture was evaporated to afford crude product. It was purified by HPLC (column: SunFire 100*19 mm, 5 microM; mobile phase: 35-50% 2-10 min H₂O/MeOH+NH₃, flow rate: 30 ml/min; loading pump 4 ml/minMeOH+NH₃) to give 2-oxo-N-(1H-pyrazolo[4,3-c]pyridin-7-yl)-2-[(2R,5S)-5-methyl-2-[2-[(1-methyl-4-piperidyl)methyl]-1,3-benzothiazol-5-yl]-1-piperidyl]acetamide (0.016 g, 30.09 μmol, 34.45% yield).

Compound 61: ¹H NMR (600 MHz, dmso) δ 0.99-1.09 (m, 3H), 1.15-1.44 (m, 4H), 1.60-1.67 (m, 2H), 1.70-1.80 (m, 2H), 1.81-1.97 (m, 3H), 2.12 (s, 3H), 2.16-2.36 (m, 1H), 2.69-2.74 (m, 2H), 2.89-3.04 (m, 3H), 3.78-4.14 (m, 1H), 5.58-5.77 (m, 1H), 7.36-7.46 (m, 1H), 7.86-7.95 (m, 1H), 7.98-8.09 (m, 1H), 8.27-8.39 (m, 1H), 8.40-8.57 (m, 1H), 8.86-9.00 (m, 1H), 11.01-11.31 (m, 1H), 12.90-13.19 (m, 1H). LCMS(ESI): [M]⁺ m/z: calcd 531.2; found 532.2; Rt=2.419 min.

### Example 21. Synthesis of N-(3-methylimidazo[1,2-a]pyridin-7-yl)-2-oxo-2-[(2R,5S)-5-methyl-2-[2-(1-methyl-4-piperidyl)-1,3-benzothiazol-5-yl]-1-piperidyl]acetamide (Compound 74)

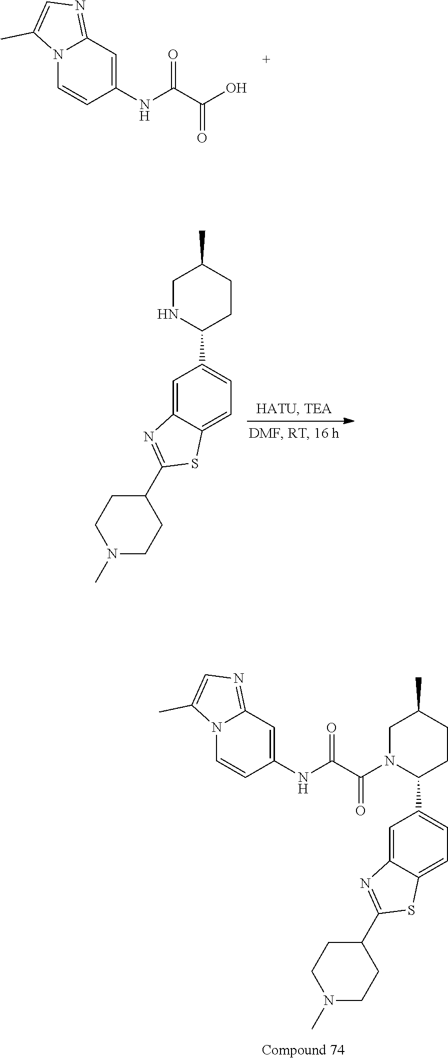

The synthesis of amine 5-((2R,5S)-5-methylpiperidin-2-yl)-2-(1-methylpiperidin-4-yl)benzo[d]thiazole is given in Intermediate 2.

The synthesis of 2-[(3-methylimidazo[1,2-a]pyridin-7-yl)amino]-2-oxo-acetic acid is given in Intermediate 34.

Prepared by general procedure scheme S2 step 6A. Yield: 37.9 mg (11.77%).

HPLC conditions: Waters Sunfire C18 20*100 5 mkm column and MeOH as an eluent mixture

Compound 74: ¹H NMR (DMSO-d6, 600 MHz): δ (ppm) 1.05 (m, 3H), 1.37 (m, 1H), 1.79 (m, 4H), 2.07 (m, 5H), 2.19 (m, 3H), 2.40 (m, 5H), 2.87 (m, 2H), 3.06 (m, 1H), 3.77 (m, 1H), 5.50 (m, 1H), 7.11 (m, 1H), 7.26 (m, 1H), 7.39 (m, 1H), 7.87 (m, 1H), 7.94 (m, 1H), 8.06 (m, 1H), 8.19 (m, 1H), 11.10 (m, 1H); LCMS(ESI): [M+1]⁺ m/z: calcd 531.2; found 531.4; Rt=2.176 min.

### Example 22. Synthesis of 2-((2R,5S)-5-methyl-2-(2-(1-(pyrrolidin-1-yl) propan-2-yl)benzo[d]thiazol-5-yl)piperidin-1-yl)-2-oxo-N-(1H-pyrazolo[4,3-c]pyridin-7-yl)acetamide (Compound 100, Compound 28 and Compound 86)

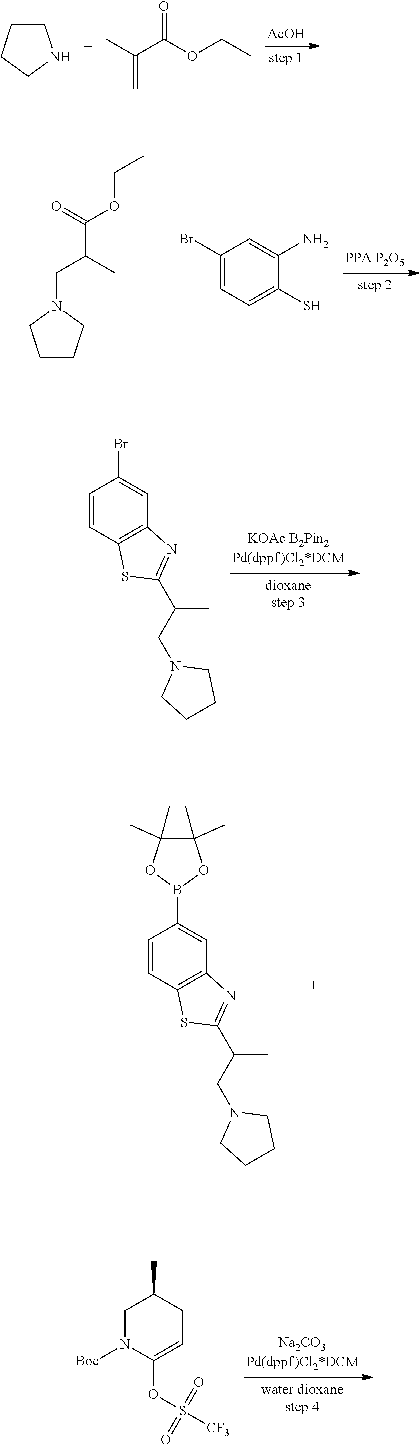

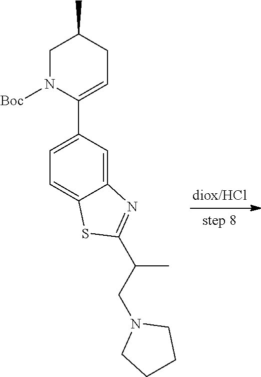 [SMILES: CC(CN1CCCC1)c1nc2cc(C3=CC[C@H](C)CN3C(=O)OC(C)(C)C)ccc2s1]

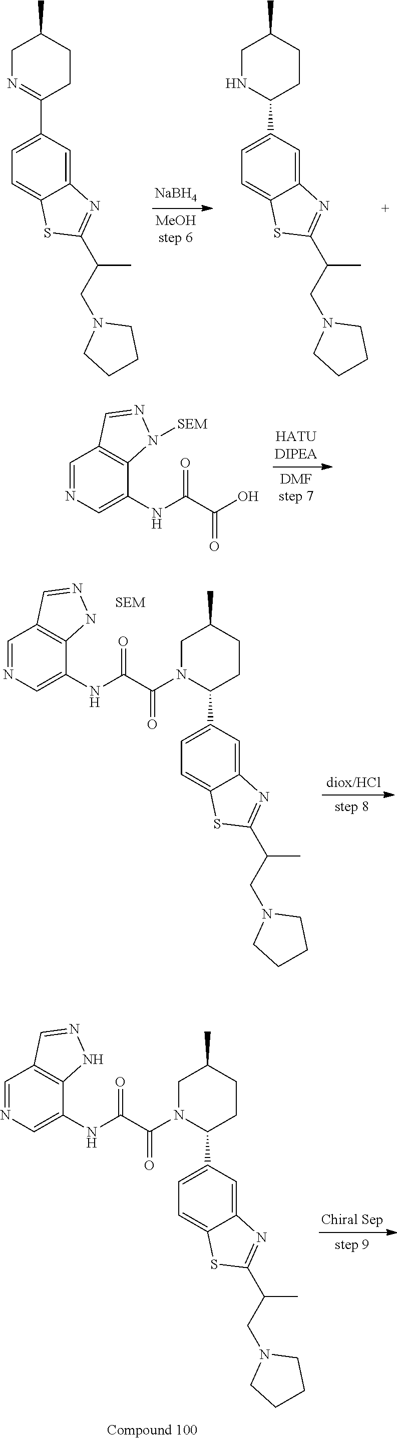

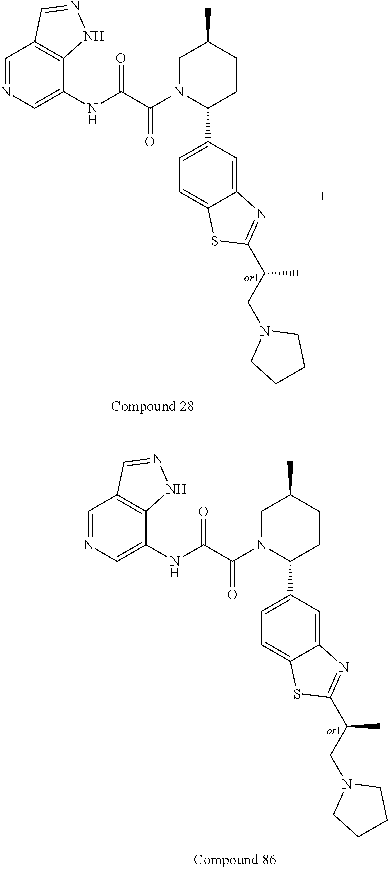 [SMILES: C[C@H]1CC[C@H](c2ccc3sc([C@H](C)CN4CCCC4)nc3c2)N(C(=O)C(=O)Nc2cncc3cn[nH]c23)C1 ; C[C@H]1CC[C@H](c2ccc3sc([C@@H](C)CN4CCCC4)nc3c2)N(C(=O)C(=O)Nc2cncc3cn[nH]c23)C1]

### Step 1: Synthesis of ethyl 2-methyl-3-(pyrrolidin-1-yl) propanoate

Pyrrolidine (7 g, 98.42 mmol, 8.18 mL) and ethyl 2-methylprop-2-enoate (10.21 g, 89.48 mmol, 11.14 mL) were mixed together and acetic acid (537.32 mg, 8.95 mmol, 512.22 μL) was added thereto. The resulting reaction mixture was stirred at 70° C. for 17 hr. After completion the reaction mixture was diluted by hexane (30 mL) and washed with NaHCO₃. This solution was filtered through the pad of silica that was washed with hexane (3*10 mL). The combined organic solution was evaporated under reduced pressure to afford ethyl 2-methyl-3-pyrrolidin-1-yl-propanoate (13 g, 70.17 mmol, 78.42% yield). ¹H NMR (400 MHZ, CDCl₃) δ (ppm)(d, 3H), 1.19 (t, 3H), 1.67 (m, 4H), 2.43 (m, 5H), 2.58 (m, 1H), 2.69 (m, 1H), 4.09 (m, 2H).

### Step 2: Synthesis of 5-bromo-2-(1-(pyrrolidin-1-yl) propan-2-yl)benzo[d]thiazole

Prepared by general procedure scheme S2 step 1A. Yield: 5 g (28.48%).

LCMS(ESI): [M]⁺ m/z: calcd 325.2; found 326.2; Rt=0.760 min.

### Step 3: Synthesis of 2-(1-(pyrrolidin-1-yl) propan-2-yl)-5-(4,4,5,5-tetramethyl-1,3,2-dioxaborolan-2-yl)benzo[d]thiazole

Prepared by general procedure scheme S2 step 2. Yield: 7.7 g of crude.

LCMS(ESI): [M]⁺ m/z: calcd 372.2; found 373.2; Rt=1.154 min.

### Step 4: Synthesis of (3S)-tert-butyl 3-methyl-6-(2-(1-(pyrrolidin-1-yl) propan-2-yl)benzo[d]thiazol-5-yl)-3,4-dihydropyridine-1(2H)-carboxylate

Prepared by general procedure scheme S2 step 3. Yield: 9 g of crude.

LCMS(ESI): [M]⁺ m/z: calcd 441.2; found 442.2; Rt=1.273 min.

### Step 5: Synthesis of 5-((S)-5-methyl-3,4,5,6-tetrahydropyridin-2-yl)-2-(1-(pyrrolidin-1-yl) propan-2-yl)benzo[d]thiazole

tert-Butyl (3S)-3-methyl-6-[2-[1-methyl-2-pyrrolidin-1-yl-ethyl]-1,3-benzothiazol-5-yl]-3,4-dihydro-2H-pyridine-1-carboxylate (4.98 g, 11.27 mmol) was dissolved in the diox/HCl (30 mL) and the resulting reaction mixture was stirred at 25° C. for 16 hr. After the completion the solvent was evaporated under reduced pressure to afford crude 2-[1-methyl-2-pyrrolidin-1-yl-ethyl]-5-[(3S)-3-methyl-1,2,3,4-tetrahydropyridin-6-yl]-1,3-benzothiazole that was directly used in the next step. LCMS(ESI): [M]⁺ m/z: calcd 341.2; found 342.2; Rt=0.678 min.

### Step 6: Synthesis of 5-((2R,5S)-5-methylpiperidin-2-yl)-2-(1-(pyrrolidin-1-yl) propan-2-yl)benzo[d]thiazole

Prepared by general procedure scheme S2 step 5. Yield: 3 g of crude.

LCMS(ESI): [M]⁺ m/z: calcd 343.2; found 344.2; Rt=0.586 min.

### Step 7: Synthesis of 2-((2R,5S)-5-methyl-2-(2-(1-(pyrrolidin-1-yl) propan-2-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

5-[(2R,5S)-5-Methyl-2-piperidyl]-2-[1-methyl-2-pyrrolidin-1-yl-ethyl]-1,3-benzothiazole (0,1 g, 291.10 μmol), 2-oxo-2-[[1-(2-trimethylsilylethoxymethyl)pyrazolo[4,3-c]pyridin-7-yl]amino]acetic acid (Intermediate 37)(97.93 mg, 291.10 μmol) and DIPEA (112.87 mg, 873.29 μmol, 152.11 μL) were dissolved in the DMF (5 mL) and mixed at 25° C. for 5 min. HATU (143.89 mg, 378.43 μmol) was added thereto and the resulting reaction mixture was stirred at 25° C. for 12 hr. After completion the reaction mixture was diluted with EtOAc (50 mL.) and washed with saturated aqueous NaCl (4*25 mL), dried over Na₂SO₄ and evaporated under reduced pressure to afford crude 2-oxo-2-[(2R,5S)-5-methyl-2-[2-[1-methyl-2-pyrrolidin-1-yl-ethyl]-1,3-benzothiazol-5-yl]-1-piperidyl]-N-[1-(2-trimethylsilylethoxymethyl)pyrazolo[4,3-c]pyridin-7-yl]acetamide (0.4 g, 604.29 μmol, 207.59% yield) that was directly used in the next step. LCMS(ESI): [M]⁺ m/z: calcd 661.2; found 662.2; Rt=1.425 min.

### Step 8: Synthesis of 2-((2R,5S)-5-methyl-2-(2-(1-(pyrrolidin-1-yl) propan-2-yl)benzo[d]thiazol-5-yl)piperidin-1-yl)-2-oxo-N-(1H-pyrazolo[4,3-c]pyridin-7-yl)acetamide (Compound 100)

2-Oxo-2-[(2R,5S)-5-methyl-2-[2-[1-methyl-2-pyrrolidin-1-yl-ethyl]-1,3-benzothiazol-5-yl]-1-piperidyl]-N-[1-(2-trimethylsilylethoxymethyl)pyrazolo[4,3-c]pyridin-7-yl]acetamide (0.4 g, 604.29 μmol) was dissolved in the diox/HCl (5 mL) and this reaction mixture was stirred at 25° C. for 12 hr. After completion the solvent was evaporated under reduced pressure and residue was purified by HPLC (Device (Mobile Phase, Column): SYSTEM 5-55% 0-5 min H₂O/MeCN/0.1% NH₄OH, flow: 30 ml/min (loading pump 4 ml/min MeCN) column: XBridge C18 100×19 mm, 5 um) to give 2-oxo-N-(1H-pyrazolo[4,3-c]pyridin-7-yl)-2-[(2R,5S)-5-methyl-2-[2-[1-methyl-2-pyrrolidin-1-yl-ethyl]-1,3-benzothiazol-5-yl]-1-piperidyl]acetamide (0.031 g, 58.31 μmol, 9.65% yield).

Compound 100: ¹H NMR (DMSO-d6, 600 MHz): δ (ppm) 1.07 (m, 3H), 1.38 (m, 4H), 1.67 (m, 4H), 1.76 (m, 1H), 1.99 (m, 2H), 2.30 (m, 2H), 2.56 (m, 2H), 2.78 (m, 3H), 3.45 (m, 2H), 3.95 (m, 1H), 5.68 (m, 1H), 7.40 (m, 1H), 7.91 (d, 1H), 8.04 (m, 1H), 8.33 (m, 1H), 8.48 (d, 1H), 8.93 (m, 1H), 11.12 (m, 1H), 13.04 (m, 1H). LCMS(ESI): [M]⁺ m/z: calcd 531.2; found 532.2; Rt=2.186 min.

### Step 9: Chiral Separation (Compound 28 and Compound 86)

Racemic 2-((2R,5S)-5-methyl-2-(2-(1-(pyrrolidin-1-yl) propan-2-yl)benzo[d]thiazol-5-yl)piperidin-1-yl)-2-oxo-N-(1H-pyrazolo[4,3-c]pyridin-7-yl)acetamide (31 mg, 58.31 μmol) was chiral separated (Column: Chiralpak IA (250*20, 5 mkm), CO₂-MeOH, 70-30, 50 ml/min) to obtain 2-oxo-N-(1H-pyrazolo[4,3-c]pyridin-7-yl)-2-[(2R,5S)-5-methyl-2-[2-[(1R)-1-methyl-2-pyrrolidin-1-yl-ethyl]-1,3-benzothiazol-5-yl]-1-piperidyl]acetamide (0.00146 g, 2.75 μmol, 4.71% yield) (RT=22.67 min) and 2-oxo-N-(1H-pyrazolo[4,3-c]pyridin-7-yl)-2-[(2R,5S)-5-methyl-2-[2-[(1S)-1-methyl-2-pyrrolidin-1-yl-ethyl]-1,3-benzothiazol-5-yl]-1-piperidyl]acetamide (0.00307 g, 5.77 μmol, 9.90% yield) (RT=13.8 min).

Rel Time for Compound 28 in analytical conditions (column: IA, CO₂-MeOH, 70-30, 3 ml/min as mobile phase) 24.57 min and for Compound 86 19.81 min.

Compound 28: Retention time: 24.57 min. ¹H NMR (DMSO-d6, 600 MHz): δ (ppm) 1.07 (m, 3H), 1.38 (m, 4H), 1.67 (m, 4H), 1.76 (m, 1H), 1.99 (m, 2H), 2.30 (m, 2H), 2.56 (m, 2H), 2.78 (m, 3H), 3.45 (m, 2H), 3.95 (m, 1H), 5.68 (m, 1H), 7.40 (m, 1H), 7.91 (d, 1H), 8.04 (m, 1H), 8.33 (m, 1H), 8.48 (d, 1H), 8.93 (m, 1H), 11.12 (m, 1H), 13.04 (m, 1H). LCMS(ESI): [M]⁺ m/z: calcd 531.2; found 532.2; Rt=2.962 min.

Compound 86: Retention time: 19.81 min. ¹H NMR (DMSO-d6, 600 MHz): δ (ppm) 1.07 (m, 3H), 1.38 (m, 4H), 1.67 (m, 4H), 1.76 (m, 1H), 1.99 (m, 2H), 2.30 (m, 2H), 2.56 (m, 2H), 2.78 (m, 3H), 3.45 (m, 2H), 3.95 (m, 1H), 5.68 (m, 1H), 7.40 (m, 1H), 7.91 (d, 1H), 8.04 (m, 1H), 8.33 (m, 1H), 8.48 (d, 1H), 8.93 (m, 1H), 11.12 (m, 1H), 13.04 (m, 1H). LCMS(ESI): [M]⁺ m/z: calcd 531.2; found 532.2; Rt=2.977 min.

### Example 23. Synthesis of 2-((2R,5S)-2-(2-(3-(dimethylamino)cyclobutyl)benzo[d]thiazol-5-yl)-5-methylpiperidin-1-yl)-2-oxo-N-(1H-pyrazolo[4,3-c]pyridin-7-yl)acetamide (Compound 64)

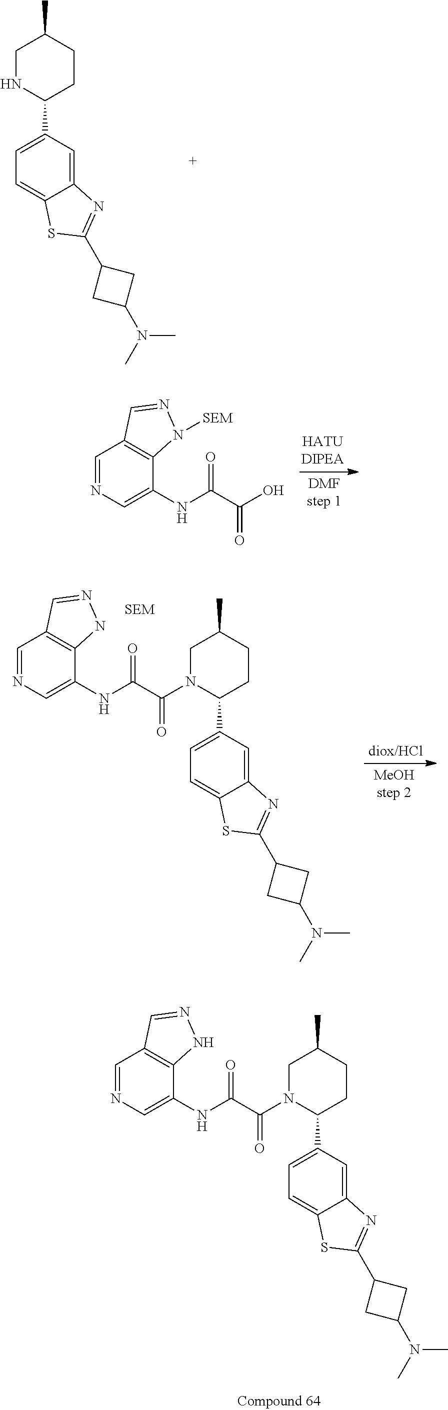

The Synthesis of N,N-dimethyl-3-(5-((2R,5S)-5-methylpiperidin-2-yl)benzo[d]thiazol-2-yl)cyclobutanamine is given in Intermediate 10

### Step 1: Synthesis of 2-((2R,5S)-2-(2-(3-(dimethylamino)cyclobutyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

To the solution of N,N-dimethyl-3-[5-[(2R,5S)-5-methyl-2-piperidyl]-1,3-benzothiazol-2-yl]cyclobutanamine (0.27 g, 819.42 μmol), 2-oxo-2-[[1-(2-trimethylsilylethoxymethyl)pyrazolo[4,3-c]pyridin-7-yl]amino]acetic acid (Intermediate 37) (275.67 mg, 819.42 μmol) and TEA (331.67 mg, 3.28 mmol, 456.84 μL) in DMF (3 mL), HATU (311.57 mg, 819.42 μmol) was added portion wise. Mixture was stirred at 25° C. for 3 hr. The reaction mixture was poured into water (25 ml) and extracted with EtOAc (3*20 ml). Combined organic layers were washed with water (3*25 ml) dried over Na₂SO₄. EtOAc was evaporated in vacuum to give 2-oxo-2-[(2R,5S)-2-[2-[3-(dimethylamino)cyclobutyl]-1,3-benzothiazol-5-yl]-5-methyl-1-piperidyl]-N-[1-(4-trimethylsilylbutyl)pyrazolo[4,3-c]pyridin-7-yl]acetamide (0.35 g, crude). LCMS(ESI): [M]⁺ m/z: calcd 645.2; found 646.2; Rt=1.146 min.

### Step 2: Synthesis of 2-((2R,5S)-2-(2-(3-(dimethylamino)cyclobutyl)benzo[d]thiazol-5-yl)-5-methylpiperidin-1-yl)-2-oxo-N-(1H-pyrazolo[4,3-c]pyridin-7-yl)acetamide (Compound 64)

To the stirred solution of the 2-oxo-2-[(2R,5S)-2-[2-[3-(dimethylamino)cyclobutyl]-1,3-benzothiazol-5-yl]-5-methyl-1-piperidyl]-N-[1-(4-trimethylsilylbutyl)pyrazolo[4,3-c]pyridin-7-yl]acetamide (0.15 g, 232.22 μmol) in MeOH (5 mL), hydrogen chloride solution 4.0M in dioxane (1.20 g, 32.91 mmol, 1.5 mL) was added. The resulting mixture was stirred at 25° C. for 15 hr. The reaction mixture was concentrated under reduce pressure. The residue was purified by reverse phase HPLC (SYSTEM 5-5-40% 0-1-6 min H₂O/MeCN/0.1% NH₄OH, flow: 30 ml/min (loading pump 4 ml/min MeOH) target mass 517 column: YMC Triart C18 100×20 mm, 5 um) to give 2-oxo-N-(1H-pyrazolo[4,3-c]pyridin-7-yl)-2-[(2R,5S)-2-[2-[3-(dimethylamino)cyclobutyl]-1,3-benzothiazol-5-yl]-5-methyl-1-piperidyl]acetamide (4.9 mg, 9.47 μmol, 4.08% yield).

Compound 64: ¹H NMR (DMSO-d6, 600 MHz): δ (ppm) 1.06 (m, 3H), 1.38 (m, 1H), 1.86 (m, 3H), 2.05 (s, 6H), 2.12 (m, 2H), 2.40 (m, 3H), 2.55 (m, 1H), 2.70 (m, 1H), 2.92 (m, 1H), 3.79 (m, 2H), 5.68 (m, 1H), 7.41 (m, 1H), 8.00 (m, 2H), 8.33 (m, 1H), 8.48 (m, 1H), 8.93 (m, 1H), 11.14 (m, 1H), 13.07 (m, 1H). LCMS(ESI): [M]⁺ m/z: calcd 517.2; found 518.2; Rt=1.766 min.

### Example 24. Synthesis of 2-((55)-5-methyl-2-(2-(1,3,3-trimethylpiperidin-4-yl)benzo[d]thiazol-5-yl)piperidin-1-yl)-2-oxo-N-(1H-pyrazolo[4,3-c]pyridin-7-yl)acetamide (Compound 81)

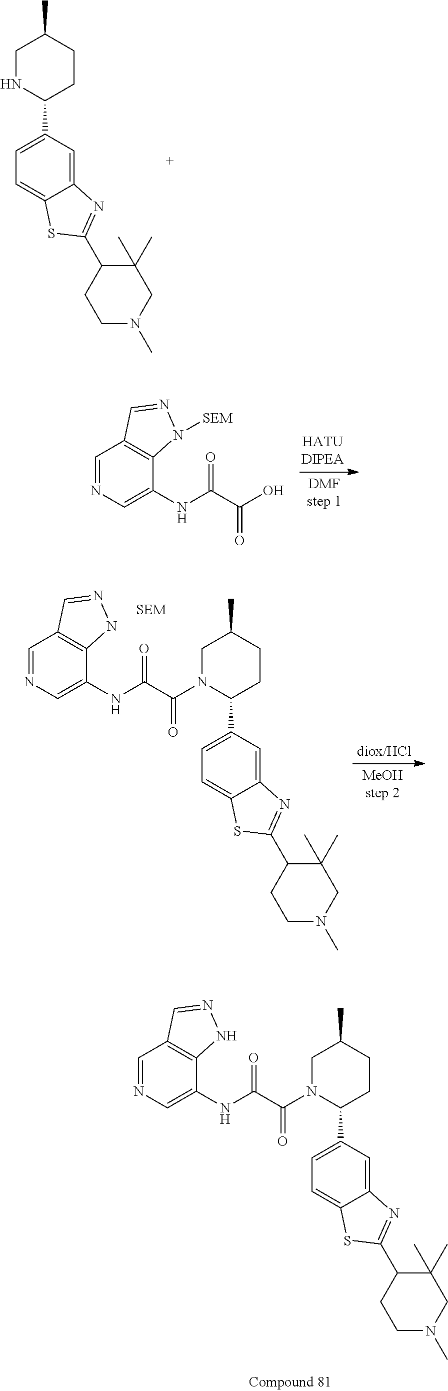 [SMILES: C[C@H]1CC[C@H](c2ccc3sc(C4CCN(C)CC4(C)C)nc3c2)NC1 ; C[Si](C)(C)CCOCn1ncc2cncc(NC(=O)C(=O)O)c21 ; C[C@H]1CC[C@H](c2ccc3sc(C4CCN(C)CC4(C)C)nc3c2)N(C(=O)C(=O)Nc2cncc3cn[nH]c23)C1 ; "SEM" ; C[C@H]1CC[C@H](c2ccc3sc(C4CCN(C)CC4(C)C)nc3c2)N(C(=O)C(=O)Nc2cncc3cn[nH]c23)C1]

The synthesis of 5-((2R,5S)-5-methylpiperidin-2-yl)-2-(1,3,3-trimethylpiperidin-4-yl)benzo[d]thiazole is given in Intermediate 11.

### Step 1: Synthesis of 2-((2R,5S)-5-methyl-2-(2-(1,3,3-trimethylpiperidin-4-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A using Intermediate 37. Yield: 28.1 mg of crude.

HPLC conditions: Column: XBridge C18 100*19 mm, 5 microM; 0-1-5 min 65-90% water-MeOH+0.1% NH₄OH, flow: 30 ml/min; (loading pump 4 ml/min MeOH).

LCMS(ESI): [M]⁺ m/z: calcd 675.2; found 676.2; Rt=3.484 min.

### Step 2: Synthesis of 2-((5S)-5-methyl-2-(2-(1,3,3-trimethylpiperidin-4-yl)benzo[d]thiazol-5-yl)piperidin-1-yl)-2-oxo-N-(1H-pyrazolo[4,3-c]pyridin-7-yl)acetamide (Compound 81)

2-Oxo-2-[(5S)-5-methyl-2-[2-(1,3,3-trimethyl-4-piperidyl)-1,3-benzothiazol-5-yl]-1-piperidyl]-N-[1-(2-trimethylsilylethoxymethyl)pyrazolo[4,3-c]pyridin-7-yl]acetamide (28.1 mg, 41.57 μmol) was dissolved in a mixture of MeOH (1 mL) and diox/HCl (1 mL), the resulting mixture was stirred for 17 hr at rt. Upon completion of the reaction, the resulting mixture was evaporated to dryness, to obtain crude product. Crude product was subjected by HPLC (column: Chromatorex 18 SMB100-5T 100×19 mm, 5 um; mobile phase: 5-30% 0-5 min H₂O/MeCN/0.1% FA, flow rate: 30 ml/min (loading pump 4 ml/min MeCN)), affording 2-oxo-N-(1H-pyrazolo[4,3-c]pyridin-7-yl)-2-[(5S)-5-methyl-2-[2-(1,3,3-trimethyl-4-piperidyl)-1,3-benzothiazol-5-yl]-1-piperidyl]acetamide (13 mg, 22.33 μmol, 53.72% yield, HCl) on two portions.

Compound 81: ¹H NMR (600 MHz, dmso) δ 0.92-0.96 (m, 3H), 0.97-1.02 (m, 3H), 1.05-1.12 (m, 3H), 1.34-1.44 (m, 1H), 1.74-1.84 (m, 3H), 1.87-1.96 (m, 2H), 2.09-2.19 (m, 5H), 2.19-2.36 (m, 2H), 2.83-2.94 (m, 3H), 3.78-4.14 (m, 1H), 5.55-5.79 (m, 1H), 7.34-7.46 (m, 1H), 7.90-7.97 (m, 1H), 8.00-8.12 (m, 1H), 8.26-8.41 (m, 1H), 8.41-8.54 (m, 1H), 8.86-9.01 (m, 1H), 11.06-11.24 (m, 1H), 13.00-13.15 (m, 1H). LCMS(ESI): [M]⁺ m/z: calcd 545.2; found 546.2; Rt=2.200 min.

### Example 25. Synthesis of N-(imidazo[1,2-a]pyridin-7-yl)-2-((2R,5S)-5-methyl-2-(2-(2-methyl-2-azabicyclo[2.2.2]octan-4-yl)benzo[d]thiazol-5-yl)piperidin-1-yl)-2-oxoacetamide (Compound 110)

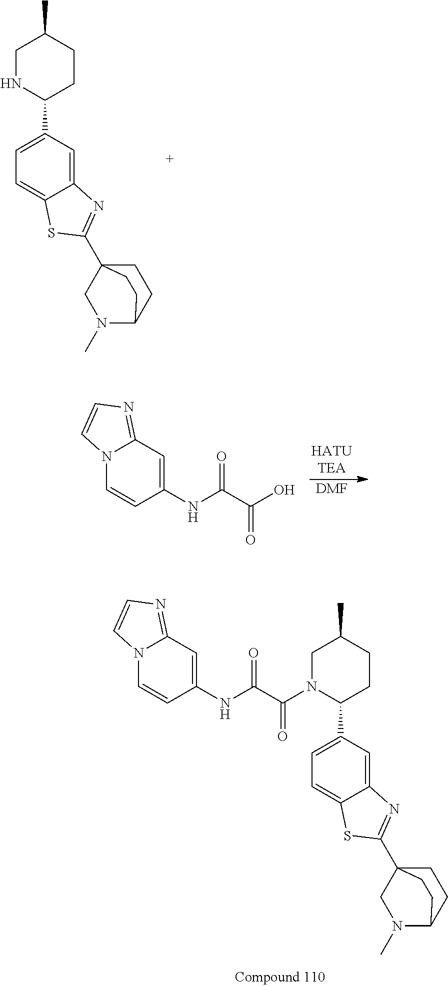

The Synthesis of 2-(2-methyl-2-azabicyclo[2.2.2]octan-4-yl)-5-((2R,5S)-5-methylpiperidin-2-yl)benzo[d]thiazole is given in Intermediate 12.

Prepared by general procedure scheme S2 step 6A. Yield: 74.6 mg (31.66%).

### HPLC Conditions:

First run: Column: XBridge C18 100×19 mm, 5 microM; 0-5 min 40-90% water-MeOH+0.1% NH₄OH, flow: 30 ml/min; (loading pump 4 ml/min MeOH).

Second run: Column: Chromatorex 18 SMB100-5T 100×19 mm, 5 microM; 0-5 min 5-30% water-MeCN+0.1% FA, flow: 30 ml/min; (loading pump 4 ml/min MeCN).

Compound 110: ¹H NMR (600 MHz, dmso) § 1.00-1.10 (m, 3H), 1.31-1.41 (m, 1H), 1.59-1.67 (m, 2H), 1.67-1.75 (m, 1H), 1.84-2.02 (m, 5H), 2.02-2.08 (m, 2H), 2.26-2.36 (m, 1H), 2.39-2.42 (m, 3H), 2.71-2.75 (m, 1H), 2.89-2.91 (m, 0.3H), 3.00-3.05 (m, 2H), 3.31-3.32 (m, 0.7H), 3.50-3.50 (m, 0.7H), 4.01-4.04 (m, 0.3H), 5.24-5.74 (m, 1H), 6.97-7.13 (m, 1H), 7.36-7.44 (m, 1H), 7.44-7.52 (m, 1H), 7.79-7.90 (m, 2H), 7.90-8.02 (m, 1H), 8.04-8.12 (m, 1H), 8.17 (s, 2H), 8.38-8.53 (m, 1H), 10.99-11.27 (m, 1H). LCMS(ESI): [M]⁺ m/z: calcd 542.2; found 543.2; Rt=1.129 min.

### Example 26. Synthesis of N-(imidazo[1,2-a]pyridin-7-yl)-2-((2R,5S)-5-methyl-2-(2-(1-methylpiperidin-4-yl)benzo[d]thiazol-5-yl)piperidin-1-yl)-2-oxoacetamide (Compound 91)

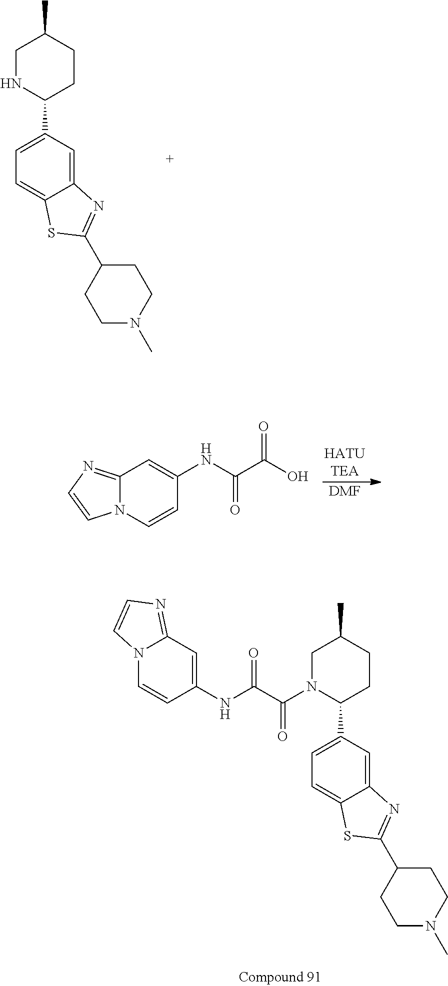

The synthesis of 5-(2R,5S)-5-methylpiperidin-2-yl)-2-(1-methylpiperidin-4-yl)benzo[d]thiazole is given in Intermediate 2.

Prepared by general procedure scheme S2 step 6A. Yield: 104 mg (25.51%).

HPLC conditions: Column: YMC Triart C18 100*20 mm, 5 microM; 0-1-5 min 20-20-60% water-MeCN+0.1% NH₄OH; (loading pump 4 ml/min MeCN).

Compound 91: ¹H NMR (600 MHZ, DMSO-d₆) δ (ppm) 1.03-1.06 (m, 3H), 1.33-1.40 (m, 1H), 1.71-1.91 (m, 4H), 2.01-2.18 (m, 8H), 2.30-2.37 (m, 1H), 2.82-2.90 (m, 2H), 3.06 (m, 1H), 3.48-4.04 (m, 2H), 5.28-5.71 (m, 1H), 7.00-7.10 (dd, 1H), 7.36-7.50 (m, 2H), 7.81-8.09 (m, 4H), 8.41-8.50 (dd, 1H), 11.07-11.20 (m, 1H). LCMS(ESI): [M]⁺ m/z: calcd 516.2; found 517.2; Rt=1.737 min.

### Example 27. Synthesis of N-(imidazo[1,2-a]pyridin-7-yl)-2-((2R,5S)-5-methyl-2-(2-(2-methyl-2-azaspiro[3.3]heptan-6-yl)benzo[d]thiazol-5-yl)piperidin-1-yl)-2-oxoacetamide (Compound 44)

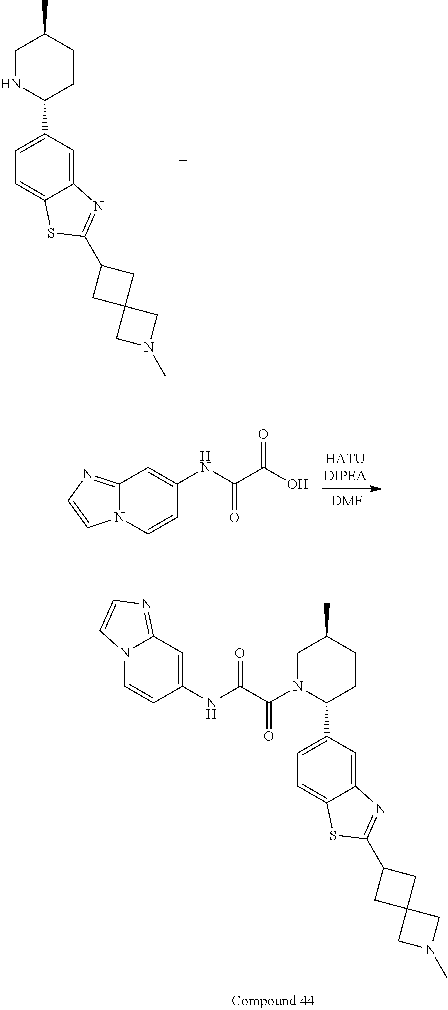

The synthesis of 2-(2-methyl-2-azaspiro[3.3]heptan-6-yl)-5-((2R,5S)-5-methylpiperidin-2-yl)benzo[d]thiazole is given in Intermediate 13.

Prepared by general procedure scheme S2 step 6A. Yield: 62 mg (16.02%).

HPLC conditions: Column: XBridge C18 100*19 mm, 5 microM; 0-1-5 min 20-60% water-MeOH+0.1% NH₄OH; (loading pump 4 ml/min MeOH).

¹H NMR (500 MHz, dmso) δ 1.01-1.07 (m, 3H), 1.29-1.43 (m, 1H), 1.65-1.76 (m, 1H), 1.82-1.94 (m, 1H), 2.05-2.13 (m, 1H), 2.17 (s, 3H), 2.19-2.35 (m, 2H), 2.55-2.60 (m, 2H), 2.84-3.03 (m, 1H), 3.04-3.12 (m, 2H), 3.20-3.27 (m, 3H), 3.45-4.06 (m, 2H), 5.25-5.75 (m, 1H), 6.98-7.12 (m, 1H), 7.33-7.42 (m, 1H), 7.44-7.53 (m, 1H), 7.80-8.07 (m, 4H), 8.40-8.53 (m, 1H), 11.04-11.28 (m, 1H). LCMS(ESI): [M]⁺ m/z: calcd 528.2; found 529.2; Rt=1.032 min.

### Example 28. Synthesis of 2-((2R,5S)-5-methyl-2-(2-(2-methyl-2-azabicyclo[2.2.2]octan-4-yl)benzo[d]thiazol-5-yl)piperidin-1-yl)-2-oxo-N-(1H-pyrazolo[4,3-c]pyridin-7-yl)acetamide (Compound 54)

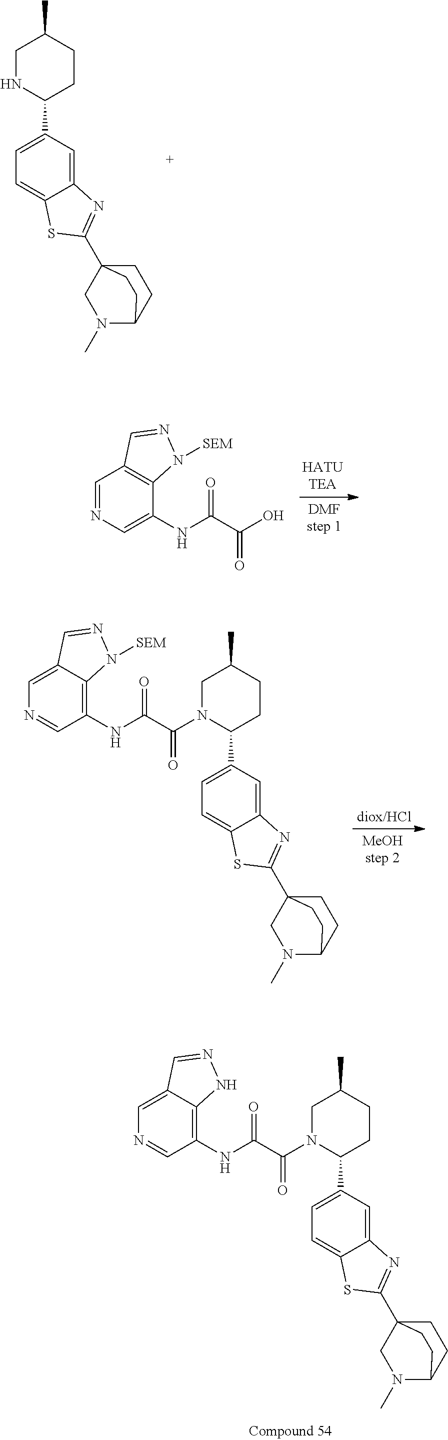 [SMILES: C[C@H]1CC[C@H](c2ccc3sc(C45CCC(CC4)N(C)C5)nc3c2)NC1 ; C[Si](C)(C)CCOCn1ncc2cncc(NC(=O)C(=O)O)c21 ; C[C@H]1CC[C@H](c2ccc3sc(C45CCC(CC4)N(C)C5)nc3c2)N(C(=O)C(=O)Nc2cncc3cnn(COCC[Si](C)(C)C)c23)C1 ; C[C@H]1CC[C@H](c2ccc3sc(C45CCC(CC4)N(C)C5)nc3c2)N(C(=O)C(=O)Nc2cncc3cn[nH]c23)C1]

The Synthesis of 2-(2-methyl-2-azabicyclo[2.2.2]octan-4-yl)-5-((2R,5S)-5-methylpiperidin-2-yl)benzo[d]thiazole is given in Intermediate 12.

### Step 1: Synthesis of 2-((2R,5S)-5-methyl-2-(2-(2-methyl-2-azabicyclo[2.2.2]octan-4-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A using Intermediate 37. Yield: 205 mg (36.66%).

HPLC conditions: Column: XBridge BEH C18 100*20 mm, 5 microM; 0-1-6 min 50-50-90% water-MeOH+0.1% NH₄OH, flow: 30 ml/min; (loading pump 4 ml/min MeOH).

LCMS(ESI): [M]⁺ m/z: calcd 673.2; found 674.2; Rt=1.078 min.

### Step 2: Synthesis of 2-((2R,5S)-5-methyl-2-(2-(2-methyl-2-azabicyclo[2.2.2]octan-4-yl)benzo[d]thiazol-5-yl)piperidin-1-yl)-2-oxo-N-(1H-pyrazolo[4,3-c]pyridin-7-yl)acetamide (Compound 54)

Hydrogen chloride solution 4.0 M in dioxane (1.01 g, 3.32 mmol, 1 mL, 12% purity) was added to the solution of 2-((2R,5S)-5-methyl-2-(2-(2-methyl-2-azabicyclo[2.2.2]octan-4-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide (205 mg) in MeOH (2 mL). Resulting mixture was stirred at 28° C. for 72 hr. Then, it was concentrated under reduced pressure and residue was purified by HPLC (30-30-80% 0-1-6 min H₂O/MeOH/0.1% NH₄OH, flow: 30 ml/min; column: XBridge BEH C18 5 um 130), affording 2-oxo-N-(1H-pyrazolo[4,3-c]pyridin-7-yl)-2-[(2R,5S)-5-methyl-2-[2-(2-methyl-2-azabicyclo[2.2.2]octan-4-yl)-1,3-benzothiazol-5-yl]-1-piperidyl]acetamide (70 mg, 113.53 μmol, 36.54% yield, 2HCl).

Compound 54: ¹H NMR (DMSO-d6, 600 MHz): δ (ppm) 1.05 (m, 3H), 1.36 (m, 1H), 1.58 (m, 2H), 1.74 (m, 1H), 1.94 (m, 8H), 2.13 (m, 1H), 2.30 (m, 3H), 2.57 (m, 1H), 2.91 (m, 3H), 3.94 (m, 1H), 5.72 (m, 1H), 7.41 (m, 1H), 7.91 (m, 1H), 8.06 (m, 1H), 8.33 (m, 1H), 8.52 (m, 1H), 8.93 (m, 1H), 11.14 (m, 1H), 13.07 (m, 1H). LCMS(ESI): [M]⁺ m/z: calcd 543.2; found 544.2; Rt=2.023 min.

### Example 29. Synthesis of 2-((2R,5S)-2-(2-(1-azabicyclo[2.2.1]heptan-4-yl)benzo[d]thiazol-5-yl)-5-methylpiperidin-1-yl)-2-oxo-N-(1H-pyrazolo[4,3-c]pyridin-7-yl)acetamide (Compound 57)

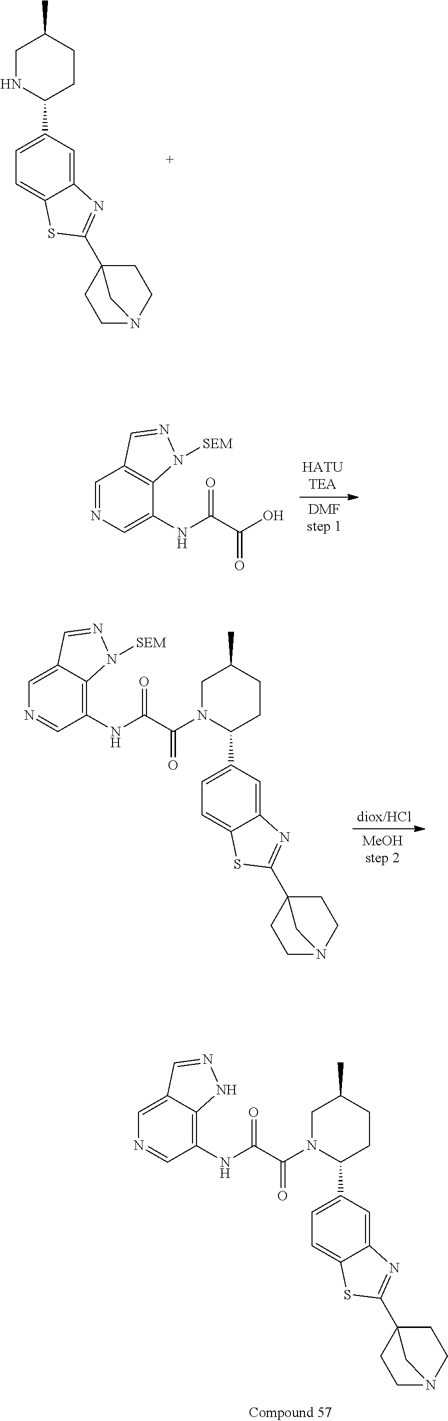 [SMILES: C[C@H]1CC[C@H](c2ccc3sc(C45CCN(CC4)C5)nc3c2)NC1 ; C[Si](C)(C)CCOCn1ncc2cncc(NC(=O)C(=O)O)c21 ; C[C@H]1CC[C@H](c2ccc3sc(C45CCN(CC4)C5)nc3c2)N(C(=O)C(=O)Nc2cncc3cnn(COCC[Si](C)(C)C)c23)C1 ; C[C@H]1CC[C@H](c2ccc3sc(C45CCN(CC4)C5)nc3c2)N(C(=O)C(=O)Nc2cncc3cn[nH]c23)C1]

The Synthesis of 2-(1-azabicyclo[2.2.1]heptan-4-yl)-5-((2R,5S)-5-methylpiperidin-2-yl)benzo[d]thiazole is given in Intermediate 14.

### Step 1: Synthesis of 2-((2R,5S)-2-(2-(1-azabicyclo[2.2.1]heptan-4-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6B using Intermediate 37. Yield: 328 mg (36.96%).

HPLC conditions: Column: XBridge BEH C18 100*20 mm, 5 microM; 0-1-6 min 55-55-70% water-MeOH+0.1% NH₄OH, flow: 30 ml/min; (loading pump 4 ml/min MeOH).

LCMS(ESI): [M]⁺ m/z: calcd 645.2; found 646.2; Rt=2.504 min.

### Step 2: Synthesis of 2-((2R,5S)-2-(2-(1-azabicyclo[2.2.1]heptan-4-yl)benzo[d]thiazol-5-yl)-5-methylpiperidin-1-yl)-2-oxo-N-(1H-pyrazolo[4,3-c]pyridin-7-yl)acetamide (Compound 57)

To a solution of 2-oxo-2-[(2R,5S)-2-[2-(1-azabicyclo[2.2.1]heptan-4-yl)-1,3-benzothiazol-5-yl]-5-methyl-1-piperidyl]-N-[1-(2-trimethylsilylethoxymethyl)pyrazolo[4,3-c]pyridin-7-yl]acetamide (328 mg, 507.83 μmol) in MeOH (2 mL) was added hydrogen chloride solution 4.0M in dioxane (1.85 g, 5.08 mmol, 2.31 mL, 10% purity) at 21° C. The resulting mixture was left to stir for 5 day. The resulting mixture was evaporated to dryness. The residue was purified by RP-HPLC (column: XBridge BEH C18 5 um 130 A; 15-15-60% 0-1-6 min H₂O/MeOH/0.1% NH₄OH, flow: 30 ml/min) to give Compound 57 2-oxo-N-(1H-pyrazolo[4,3-c]pyridin-7-yl)-2-[(2R,5S)-2-[2-(1-azabicyclo[2.2.1]heptan-4-yl)-1,3-benzothiazol-5-yl]-5-methyl-1-piperidyl]acetamide (32 mg, 62.06 μmol, 12.22% yield).

Compound 57: ¹H NMR (600 MHz, dmso) δ 1.03-1.09 (m, 3H), 1.34-1.45 (m, 1H), 1.69-1.79 (m, 3H), 1.84-1.98 (m, 1H), 2.04-2.21 (m, 3H), 2.24-2.36 (m, 1H), 2.63-2.70 (m, 4H), 2.89-3.19 (m, 3H), 3.79-4.11 (m, 1H), 5.53-5.79 (m, 1H), 7.37-7.48 (m, 1H), 7.86-7.95 (m, 1H), 8.01-8.13 (m, 1H), 8.28-8.38 (m, 1H), 8.40-8.56 (m, 1H), 8.83-9.01 (m, 1H), 11.00-11.28 (m, 1H), 12.92-13.28 (m, 1H). LCMS(ESI): [M]⁺ m/z: calcd 515.2; found 516.2; Rt=2.221 min.

### Example 30. Synthesis of N-(8-amino-2,7-naphthyridin-4-yl)-2-((2R,5S)-5-methyl-2-(2-(2-methyl-2-azaspiro[3.3]heptan-6-yl)benzo[d]thiazol-5-yl)piperidin-1-yl)-2-oxoacetamide (Compound 108)

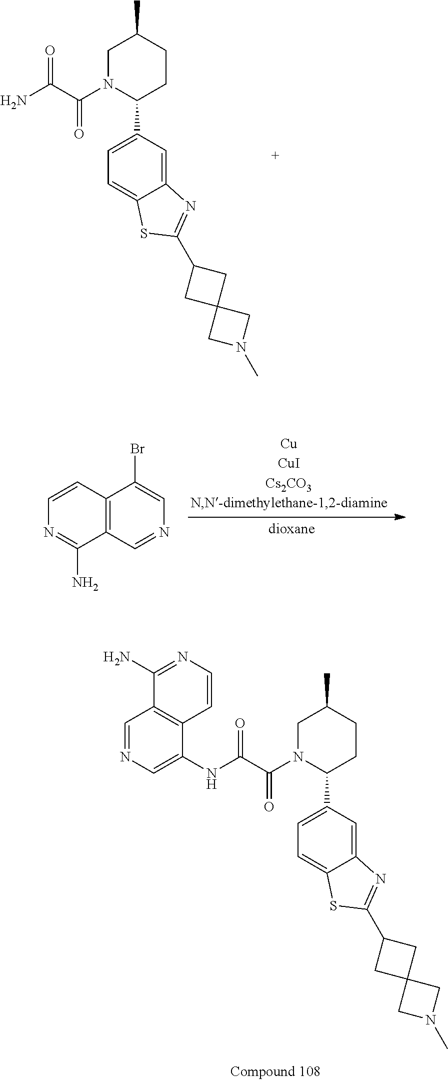 [SMILES: C[C@H]1CC[C@H](c2ccc3sc(C4CC5(C4)CN(C)C5)nc3c2)N(C(=O)C(N)=O)C1 ; Nc1nccc2c(Br)cncc12 ; C[C@H]1CC[C@H](c2ccc3sc(C4CC5(C4)CN(C)C5)nc3c2)N(C(=O)C(=O)Nc2cncc3c(N)nccc23)C1]

The synthesis of 2-((2R,5S)-5-methyl-2-(2-(2-methyl-2-azaspiro[3.3]heptan-6-yl)benzo[d]thiazol-5-yl)piperidin-1-yl)-2-oxoacetamide is given in Intermediate 26.

2-Oxo-2-[(2R,5S)-5-methyl-2-[2-(2-methyl-2-azaspiro[3.3]heptan-6-yl)-1,3-benzothiazol-5-yl]-1-piperidyl]acetamide (250 mg, 605.99 μmol), 5-bromo-2,7-naphthyridin-1-amine (162.93 mg, 727.19 μmol), copper (7.70 mg, 121.20 μmol), copper (I) iodide (34.62 mg, 181.80 μmol, 6.16 μL), (S,S)—(+)—N,N′-dimethyl-1,2-cyclohexanediamine (25.86 mg, 181.80 μmol, 28.67 μL) and potassium carbonate (167.51 mg, 1.21 mmol, 73.15 μL) were mixed together in dioxane (4 mL) and DMSO (1 mL). Reaction flask was purged with argon and resulting mixture was stirred at 105° C. for 20 hr under inert atmosphere. Then, dioxane was removed under reduced pressure and residue was subjected to HPLC (1-st run: 30-80% 0-5 min H₂O/MeOH/0.1% NH₄OH, flow: 30 ml/min; column: XBridge C18 100×19 mm, 5 um; 2-nd run: 10-35% 0-5 min H₂O/MeCN/0.1% NH₄OH, flow: 30 ml/min; column: XBridge C18 100×19 mm, 5 um; 3-rd run: 5-5-30% 0-1-5 min H₂O/MeCN/0.1% FA, flow: 30 ml/min; column: Chromatorex 18 SMB100-5T 100×19 mm 5 um), affording N-(8-amino-2,7-naphthyridin-4-yl)-2-oxo-2-[(2R,5S)-5-methyl-2-[2-(2-methyl-2-azaspiro[3.3]heptan-6-yl)-1,3-benzothiazol-5-yl]-1-piperidyl]acetamide (33.7 mg, 52.03 μmol, 8.59% yield, 2HCOOH).

Compound 108: ¹H NMR (DMSO-d6, 600 MHz): δ (ppm) 1.09 (m, 3H), 1.37 (m, 1H), 1.73 (m, 1H), 1.92 (m, 1H), 2.22 (m, 2H), 2.34 (m, 5H), 2.63 (m, 2H), 2.91 (m, 2H), 3.90 (m, 3H), 5.51 (m, 1H), 6.79 (m, 1H), 7.36 (m, 3H), 7.85 (m, 1H), 8.03 (m, 2H), 8.20 (s, 2H), 8.69 (m, 1H), 9.38 (m, 1H), 10.92 (m, 1H). LCMS(ESI): [M]⁺ m/z: calcd 555.2; found 556.2; Rt=2.103 min.

### Example 31. Synthesis of N-(4-amino-1H-pyrazolo[4,3-c]pyridin-7-yl)-2-((2R,5S)-2-(3-chlorophenyl)-5-methylpiperidin-1-yl)-2-oxoacetamide (Compound 80)

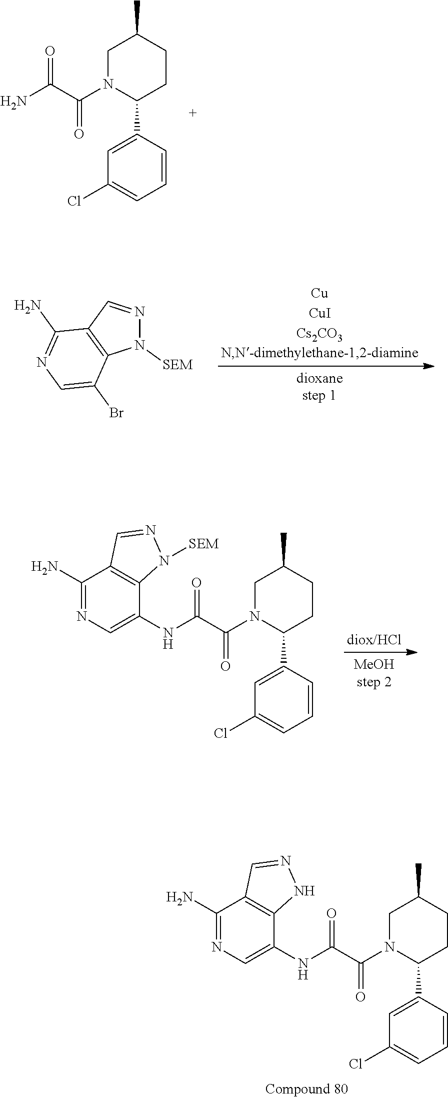 [SMILES: C[C@H]1CC[C@H](c2cccc(Cl)c2)N(C(=O)C(N)=O)C1 ; C[Si](C)(C)CCOCn1ncc2c(N)ncc(Br)c21 ; C[C@H]1CC[C@H](c2cccc(Cl)c2)N(C(=O)C(=O)Nc2cnc(N)c3cnn(COCC[Si](C)(C)C)c23)C1 ; C[C@H]1CC[C@H](c2cccc(Cl)c2)N(C(=O)C(=O)Nc2cnc(N)c3cn[nH]c23)C1]

The synthesis of 2-oxo-2-[(2R,5S)-2-(3-chlorophenyl)-5-methyl-1-piperidyl]acetamide is given in Intermediate 27.

### Step 1: Synthesis of N-(4-amino-1-(tetrahydro-2H-pyran-2-yl)-1H-pyrazolo[4,3-c]pyridin-7-yl)-2-((2R,5S)-2-(3-chlorophenyl)-5-methylpiperidin-1-yl)-2-oxoacetamide

2-Oxo-2-[(2R,5S)-2-(3-chlorophenyl)-5-methyl-1-piperidyl]acetamide (210 mg, 748.00 μmol), 7-bromo-1-(2-trimethylsilylethoxymethyl)pyrazolo[4,3-c]pyridin-4-amine (256.78 mg, 748.00 μmol), copper (I) iodide (142.46 mg, 748.00 μmol, 25.35 μL), cesium carbonate (487.43 mg, 1.50 mmol) and (1R,2R)—N1,N2-dimethylcyclohexane-1,2-diamine (159.59 mg, 1.12 mmol) were mixed in dioxane (5 mL) under argon, and then stirred overnight at 100° C. for 16 hr in vial. The reaction mixture was filtered and the filtrate was concentrated in vacuum and the residue was purified by HPLC (SYSTEM 2-10 min 30-60% MeOH+FA 30 ml/min (loading pump 4 ml MeOH) column: SunFire 100*19 mm, 5 microM) to give N-[4-amino-1-(2-trimethylsilylethoxymethyl)pyrazolo[4,3-c]pyridin-7-yl]-2-oxo-2-[(2R,5S)-2-(3-chlorophenyl)-5-methyl-1-piperidyl]acetamide (50 mg, 92.06 μmol, 12.31% yield).

LCMS(ESI): [M]⁺ m/z: calcd 543.2; found 544.2; Rt=1.459 min.

### Step 2: Synthesis of N-(4-amino-1H-pyrazolo[4,3-c]pyridin-7-yl)-2-((2R,5S)-2-(3-chlorophenyl)-5-methylpiperidin-1-yl)-2-oxoacetamide (Compound 80)

To a solution of N-[4-amino-1-(2-trimethylsilylethoxymethyl)pyrazolo[4,3-c]pyridin-7-yl]-2-oxo-2-[(2R,5S)-2-(3-chlorophenyl)-5-methyl-1-piperidyl]acetamide (64 mg, 117.84 μmol) in MeOH (5.00 mL) was added hydrogen chloride solution 4.0M in dioxane (800.00 mg, 21.94 mmol, 1.00 mL) at 25° C. The resulting mixture was left to stir for 14 hr. The resulting mixture was evaporated to dryness and then submitted to reverse phase HPLC (SYSTEM 2-10 min 45-65% MeOH+FA flow 30 ml/min (loading pump 4 ml MeOH), column: SunFire C18) to afford N-(4-amino-1H-pyrazolo[4,3-c]pyridin-7-yl)-2-oxo-2-[(2R,5S)-2-(3-chlorophenyl)-5-methyl-1-piperidyl]acetamide (12.7 mg, 27.68 μmol, 23.49% yield, HCOOH).

Compound 80: ¹H NMR (600 MHz, DMSO-d₆) δ (ppm) 1.01-1.08 (m, 3H), 1.27-1.42 (m, 1H), 1.57-1.78 (m, 1H), 1.82-1.96 (m, 1H), 2.01-2.28 (m, 2H), 2.67-2.87 (m, 1H), 3.75-4.10 (m, 1H), 5.00-5.72 (m, 1H), 6.61-6.93 (m, 2H), 6.97-7.45 (m, 4H), 7.49-7.74 (m, 1H), 8.14-8.22 (m, 1H), 10.44-10.64 (m, 1H), 12.59-12.81 (m, 1H).

LCMS(ESI): [M]⁺ m/z: calcd 412.2; found 413.2; Rt=0.988 min.

### Example 32. Synthesis of N-(8-amino-2,7-naphthyridin-4-yl)-2-((2R,5S)-5-methyl-2-(2-(1,2,2-trimethylpiperidin-4-yl)benzo[d]thiazol-5-yl)piperidin-1-yl)-2-oxoacetamide (Compound 103 and Compound 68)

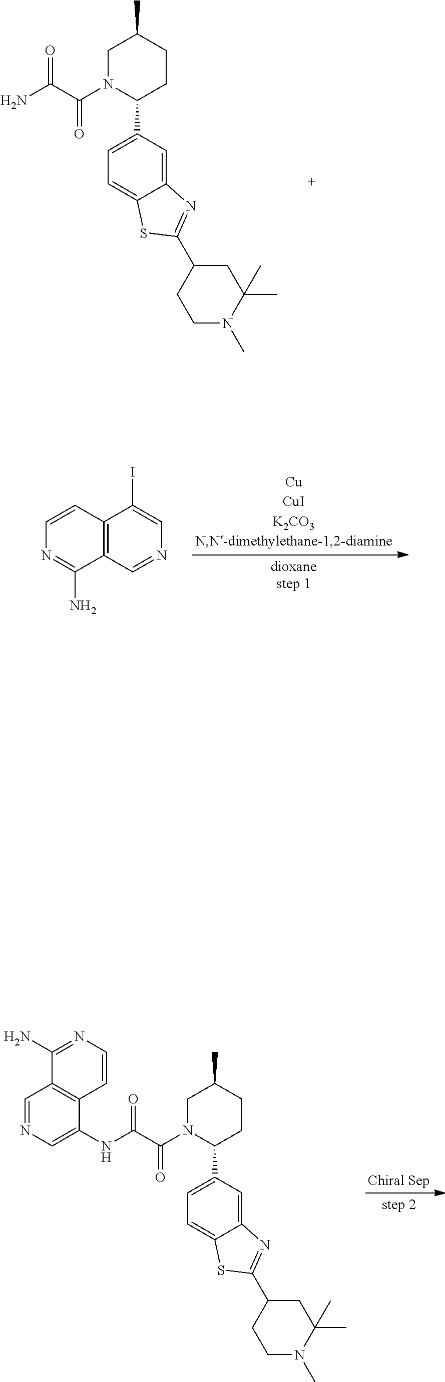 [SMILES: C[C@H]1CC[C@H](c2ccc3sc(C4CCN(C)C(C)(C)C4)nc3c2)N(C(=O)C(N)=O)C1 ; Nc1nccc2c(I)cncc12 ; C[C@H]1CC[C@H](c2ccc3sc(C4CCN(C)C(C)(C)C4)nc3c2)N(C(=O)C(=O)Nc2cncc3c(N)nccc23)C1]

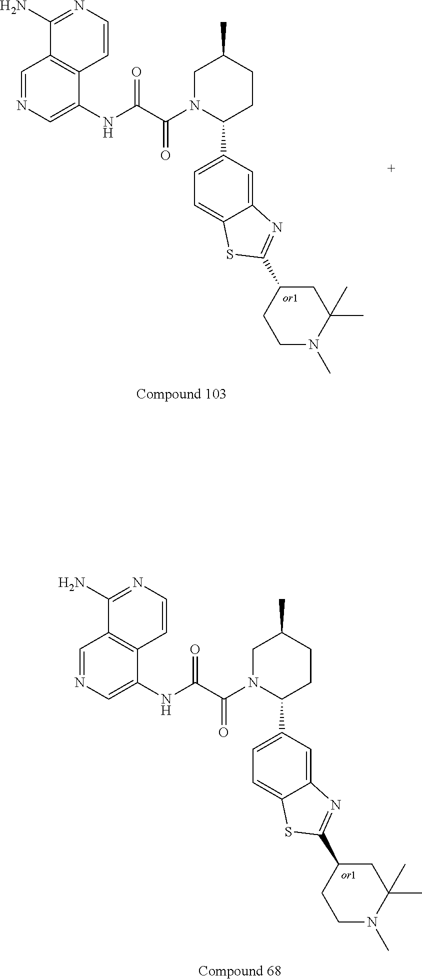 [SMILES: C[C@H]1CC[C@H](c2ccc3sc([C@H]4CCN(C)C(C)(C)C4)nc3c2)N(C(=O)C(=O)Nc2cncc3c(N)nccc23)C1 ; C[C@H]1CC[C@H](c2ccc3sc([C@@H]4CCN(C)C(C)(C)C4)nc3c2)N(C(=O)C(=O)Nc2cncc3c(N)nccc23)C1]

Synthesis of 2-((2R,5S)-5-methyl-2-(2-(1,2,2-trimethylpiperidin-4-yl)benzo[d]thiazol-5-yl)piperidin-1-yl)-2-oxoacetamide is given in Example 33.

### Step 1: Synthesis of N-(8-amino-2,7-naphthyridin-4-yl)-2-((2R,5S)-5-methyl-2-(2-(1,2,2-trimethylpiperidin-4-yl)benzo[d]thiazol-5-yl)piperidin-1-yl)-2-oxoacetamide

Prepared by general procedure Scheme S3 step 3A. Yield: 32.3 mg (24.21%).

HPLC conditions: Column: YMC Triart C18 100*20 mm, 5 microM; 0-1-6 min 30-30-80% water-MeOH+0.1% NH₄OH, flow: 30 ml/min; (loading pump 4 ml/min MeOH).

LCMS(ESI): [M]⁺ m/z: calcd 571.2; found 572.2; Rt=1.918 min.

### Step 2: Chiral Separation (Compound 103 and Compound 68)

Racemic N-(8-amino-2,7-naphthyridin-4-yl)-2-oxo-2-[(2R,5S)-5-methyl-2-[2-(1,2,2-trimethyl-4-piperidyl)-1,3-benzothiazol-5-yl]-1-piperidyl]acetamide (32.30 mg, 56.49 μmol) was chiral separated (Column: Chiralpak IA-III (250-20 mm-5 m); Mobile phase: Hexane-IPA-MeOH, 50-50 Flow Rate: 12 mL/min) to obtain N-(8-amino-2,7-naphthyridin-4-yl)-2-oxo-2-[(2R,5S)-5-methyl-2-[2-[(4S)-1,2,2-trimethyl-4-piperidyl]-1,3-benzothiazol-5-yl]-1-piperidyl]acetamide (0.0105 g, 18.37 μmol, 32.51% yield) and N-(8-amino-2,7-naphthyridin-4-yl)-2-oxo-2-[(2R,5S)-5-methyl-2-[2-[(4R)-1,2,2-trimethyl-4-piperidyl]-1,3-benzothiazol-5-yl]-1-piperidyl]acetamide (0.01337 g, 23.38 μmol, 41.39% yield).

Rel Time for Compound 103 in analytical conditions (column: OD-H, Hexane-MeOH-IPA, 50-25-25, 0.6 ml/min as mobile phase) 14.27 min and for Compound 68 15.70 min.

Compound 103: Retention time: 14.27 min. ¹H NMR (500 MHZ, DMSO-d₆) δ (ppm) 1.04 (m, 11H), 1.37 (m, 1H), 1.66 (m, 4H), 1.87 (m, 3H), 2.03 (m, 1H), 2.15 (s, 5H), 4.01 (m, 1H), 5.51 (m, 1H), 6.81 (m, 1H), 7.42 (m, 3H), 7.97 (m, 3H), 8.71 (m, 1H), 9.39 (m, 1H), 10.96 (s, 1H). LCMS(ESI): [M]⁺ m/z: calcd 571.2; found 572.2; Rt=2.128 min.

Compound 68: Retention time: 15.70 min. ¹H NMR (500 MHZ, DMSO-d₆) δ (ppm) 1.08 (m, 9H), 1.38 (m, 1H), 1.73 (m, 3H), 1.90 (m, 2H), 2.13 (m, 5H), 2.83 (m, 2H), 4.26 (m, 4H), 5.51 (m, 1H), 6.79 (m, 1H), 7.36 (m, 3H), 8.01 (m, 3H), 8.69 (m, 1H), 9.38 (m, 1H), 10.91 (m, 1H). LCMS(ESI): [M]⁺ m/z: calcd 571.2; found 572.2; Rt=2.131 min.

### Example 33. Synthesis of 2-((2R,5S)-5-methyl-2-(2-(1,2,2-trimethylpiperidin-4-yl)benzo[d]thiazol-5-yl)piperidin-1-yl)-2-oxo-N-(1H-pyrazolo[4,3-c]pyridin-7-yl)acetamide (Compound 59)

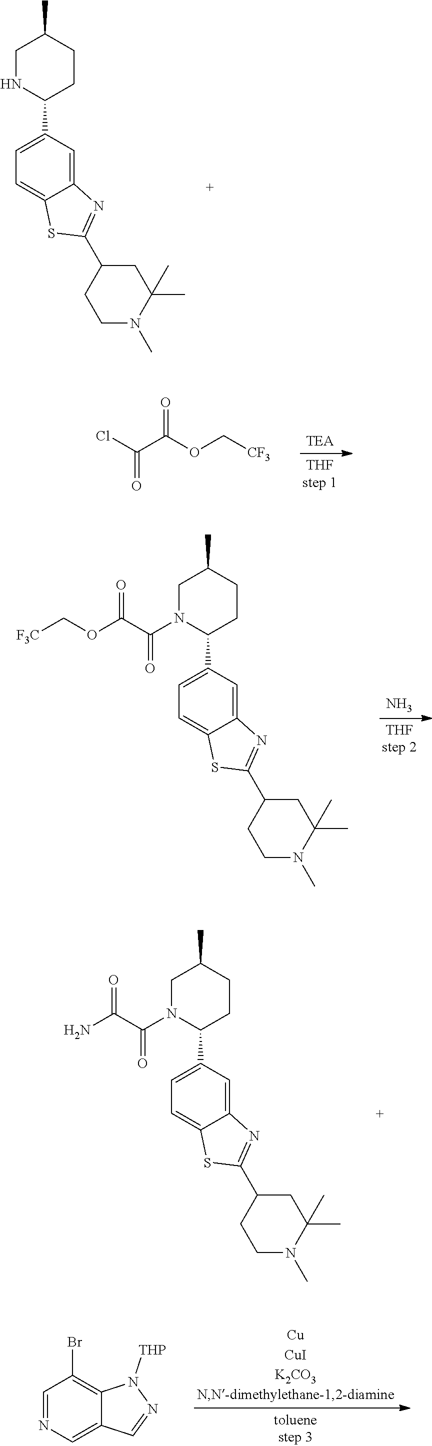

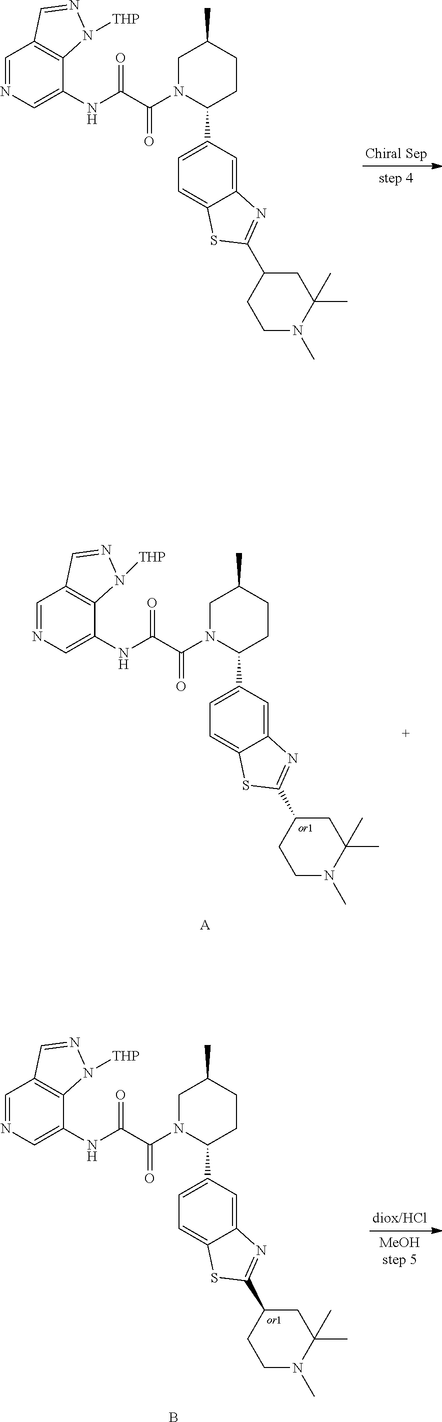

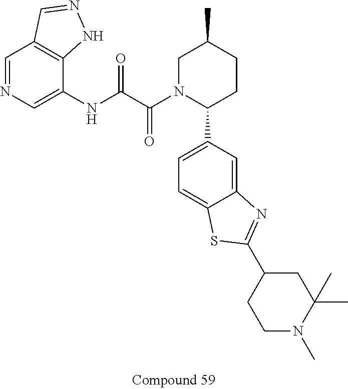 [SMILES: C[C@H]1CC[C@H](c2ccc3sc(C4CCN(C)C(C)(C)C4)nc3c2)N(C(=O)C(=O)Nc2cncc3cn[nH]c23)C1]

Synthesis of 5-((2R,5S)-5-methylpiperidin-2-yl)-2-(1,2,2-trimethylpiperidin-4-yl)benzo[d]thiazole is given in Intermediate 33.

### Step 1: Synthesis of 2,2,2-trifluoroethyl 2-((2R,5S)-5-methyl-2-(2-(1,2,2-trimethylpiperidin-4-yl)benzo[d]thiazol-5-yl)piperidin-1-yl)-2-oxoacetate

Prepared by general procedure Scheme S3 step 1. Yield: 0.46 g of crude.

LCMS(ESI): [M]⁺ m/z: calcd 511.2; found 512.2; Rt=2.623 min.

### Step 2: Synthesis of 2-((2R,5S)-5-methyl-2-(2-(1,2,2-trimethylpiperidin-4-yl)benzo[d]thiazol-5-yl)piperidin-1-yl)-2-oxoacetamide

Prepared by general procedure Scheme S3 step 2. Yield: 0.18 g of crude.

LCMS(ESI): [M]⁺ m/z: calcd 428.2; found 429.2; Rt=2.235 min.

### Step 3: Synthesis of 2-((2R,5S)-5-methyl-2-(2-(1,2,2-trimethylpiperidin-4-yl)benzo[d]thiazol-5-yl)piperidin-1-yl)-2-oxo-N-(1-(tetrahydro-2H-pyran-2-yl)-1H-pyrazolo[4,3-c]pyridin-7-yl)acetamide

7-Bromo-1-tetrahydropyran-2-yl-pyrazolo[4,3-c]pyridine (85.58 mg, 303.32 μmol), 2-oxo-2-[(2R,5S)-5-methyl-2-[2-(1,2,2-trimethyl-4-piperidyl)-1,3-benzothiazol-5-yl]-1-piperidyl]acetamide (0.1 g, 233.32 μmol), Cu (1.48 mg, 23.33 μmol), CuI (4.44 mg, 23.33 μmol, 7.91e-1 μL), potassium carbonate (64.50 mg, 466.65 μmol, 28.16 μL) and (1R,2R)—N1,N2-dimethylcyclohexane-1,2-diamine (6.64 mg, 46.66 μmol) were mixed in toluene (15 mL), purged with Ar for 15 min and then heated in the sealed tube at 100° C. for 12 hr. Final mixture was filtered and toluene was evaporated in vacuum. The crude product 0.15 g was purified by RP-HPLC (column: XBridge BEH C18 5 um 130 A; 55-55-90% 0-1-6 min H₂O/MeOH/0.1% NH₄OH, flow: 30 ml/min) to give 2-oxo-2-[(2R,5S)-5-methyl-2-[2-(1,2,2-trimethyl-4-piperidyl)-1,3-benzothiazol-5-yl]-1-piperidyl]-N-(1-tetrahydropyran-2-ylpyrazolo[4,3-c]pyridin-7-yl)acetamide (0.054 g, 85.74 μmol, 36.75% yield). LCMS(ESI): [M]⁺ m/z: calcd 629.2; found 630.2; Rt=2.158 min.

### Step 4: Chiral Separation

Racemic 2-oxo-2-[(2R,5S)-5-methyl-2-[2-(1,2,2-trimethyl-4-piperidyl)-1,3-benzothiazol-5-yl]-1-piperidyl]-N-(1-tetrahydropyran-2-ylpyrazolo[4,3-c]pyridin-7-yl)acetamide (0.074 g, 117.50 μmol) was chiral separated (Column: Chiralpak IA-III (250-20 mm-5 m); Mobile phase: Hexane-IPA-MeOH, 70-15-15 Flow Rate: 12 mL/min) to obtain 2-oxo-2-[(2R,5S)-5-methyl-2-[2-[(4R)-1,2,2-trimethyl-4-piperidyl]-1,3-benzothiazol-5-yl]-1-piperidyl]-N-(1-tetrahydropyran-2-ylpyrazolo[4,3-c]pyridin-7-yl)acetamide (0.01853 g, 29.42 μmol, 25.04% yield) and 2-oxo-2-[(2R,5S)-5-methyl-2-[2-[(4S)-1,2,2-trimethyl-4-piperidyl]-1,3-benzothiazol-5-yl]-1-piperidyl]-N-(1-tetrahydropyran-2-ylpyrazolo[4,3-c]pyridin-7-yl)acetamide (0.01497 g, 23.77 μmol, 20.23% yield).

Rel Time for A in analytical conditions (column: OD-H, Hexane-MeOH-IPA, 70-15-15, 0.6 ml/min as mobile phase) 22.88 min and for B 28.51 min.

A: Retention time: 22.88 min.

LCMS(ESI): [M]⁺ m/z: calcd 629.2; found 630.2; Rt=1.017 min.

B: Retention time: 28.51 min.

LCMS(ESI): [M]⁺ m/z: calcd 629.2; found 630.2; Rt=1.021 min.

### Step 5: Synthesis of 2-((2R,5S)-5-methyl-2-(2-(1,2,2-trimethylpiperidin-4-yl)benzo[d]thiazol-5-yl)piperidin-1-yl)-2-oxo-N-(1H-pyrazolo[4,3-c]pyridin-7-yl)acetamide (Compound 59)

To a solution of 2-oxo-2-[(2R,5S)-5-methyl-2-[2-[(4R)-1,2,2-trimethyl-4-piperidyl]-1,3-benzothiazol-5-yl]-1-piperidyl]-N-(1-tetrahydropyran-2-ylpyrazolo[4,3-c]pyridin-7-yl)acetamide (0.01853 g, 29.42 μmol) and to a solution of 2-oxo-2-[(2R,5S)-5-methyl-2-[2-[(4S)-1,2,2-trimethyl-4-piperidyl]-1,3-benzothiazol-5-yl]-1-piperidyl]-N-(1-tetrahydropyran-2-ylpyrazolo[4,3-c]pyridin-7-yl)acetamide (0.01497 g, 23.77 μmol) in MeOH (4 mL) were added hydrogen chloride solution 4.0M in dioxane (800.00 mg, 21.94 mmol, 1 mL) at 21° C. The resulting mixture was left to stir for 7 hr. The resulting mixture was evaporated to dryness and subjected to HPLC 0-6 min H₂O/MeOH/0.1% NH₄OH flow: 30 ml/min; column: YMC Triart C18 100×20 mm 5 um) to afford 2-oxo-N-(1H-pyrazolo[4,3-c]pyridin-7-yl)-2-[(2R,5S)-5-methyl-2-[2-[rac-(4R)-1,2,2-trimethyl-4-piperidyl]-1,3-benzothiazol-5-yl]-1-piperidyl]acetamide (0.0057 g, 10.45 μmol, 35.50% yield) and 2-oxo-N-(1H-pyrazolo[4,3-c]pyridin-7-yl)-2-[(2R,5S)-5-methyl-2-[2-[rac-(4S)-1,2,2-trimethyl-4-piperidyl]-1,3-benzothiazol-5-yl]-1-piperidyl]acetamide (0.0072 g, 11.64 μmol, 48.97% yield).

The compound was obtained as racemate as a result of complete racemization during HCl deprotection step.

Compound 59: ¹H NMR (600 MHZ, DMSO-d₆) δ (ppm). LCMS(ESI): [M]⁺ m/z: calcd 545.2; found 546.2; Rt=2.257 min.

### Example 34. Synthesis of N-(4-amino-1H-pyrazolo[4,3-c]pyridin-7-yl)-2-((2R,5S)-2-(2-(1,3-dimethylpiperidin-4-yl)benzo[d]thiazol-5-yl)-5-methylpiperidin-1-yl)-2-oxoacetamide (Compound 31)

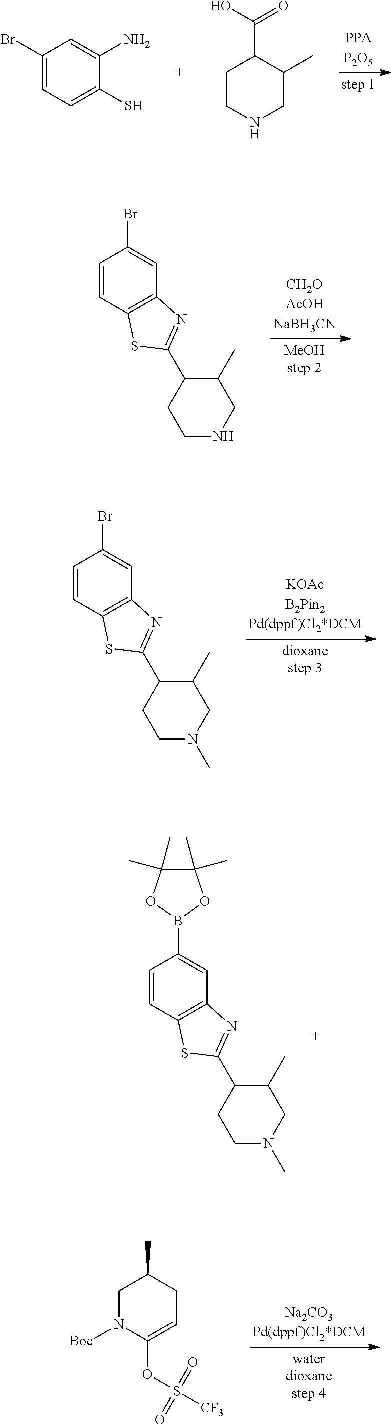

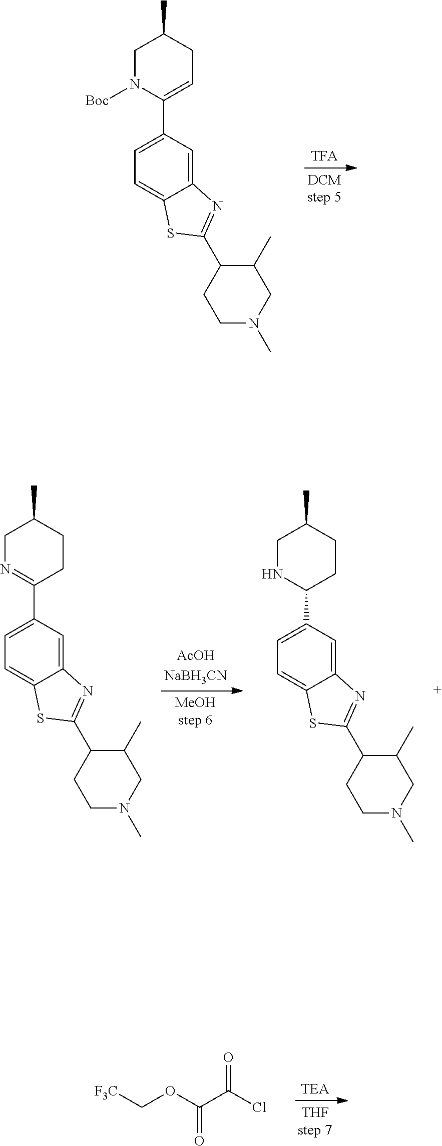 [SMILES: CC1CN(C)CCC1c1nc2cc(C3=CC[C@H](C)CN3C(=O)OC(C)(C)C)ccc2s1 ; CC1CN(C)CCC1c1nc2cc(C3=NC[C@@H](C)CC3)ccc2s1 ; CC1CN(C)CCC1c1nc2cc([C@H]3CC[C@H](C)CN3)ccc2s1 ; O=C(Cl)C(=O)OCC(F)(F)F]

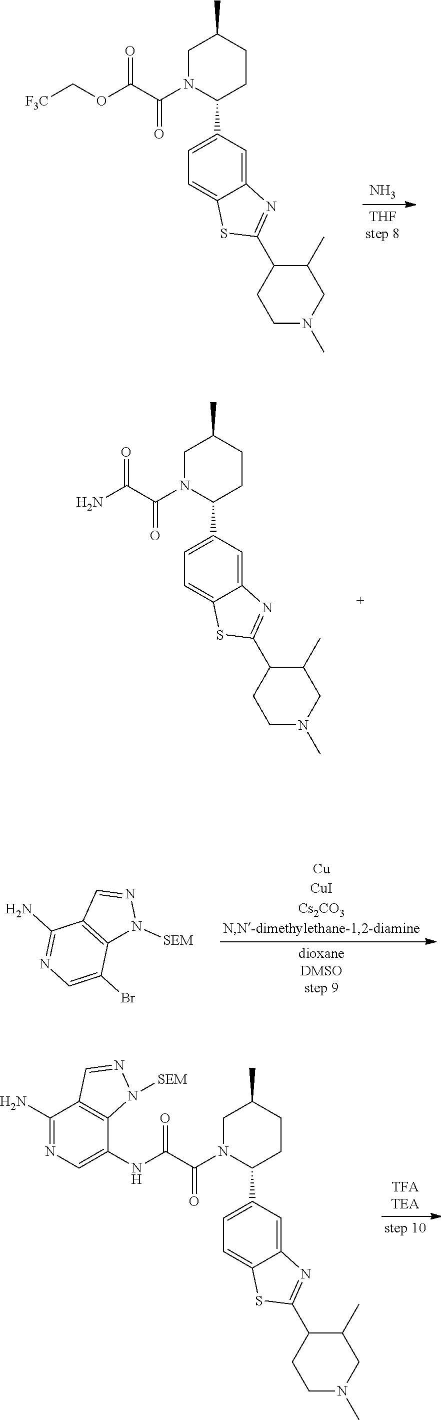

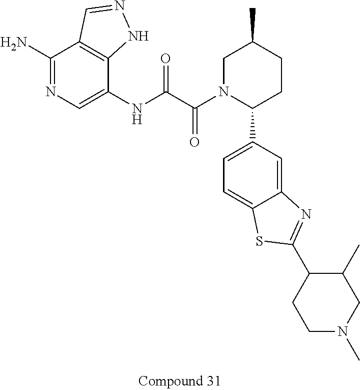 [SMILES: CC1CN(C)CCC1c1nc2cc([C@H]3CC[C@H](C)CN3C(=O)C(=O)Nc3cnc(N)c4cn[nH]c34)ccc2s1]

### Step 1: Synthesis of 5-bromo-2-(3-methylpiperidin-4-yl)benzo[d]thiazole

Prepared by general procedure scheme S2 step 1A. Yield: 5.3 g (98.97%).

LCMS(ESI): [M]⁺ m/z: calcd 311.2; found 312.2; Rt=2.406 min.

### Step 2: Synthesis of 5-bromo-2-(1,3-dimethylpiperidin-4-yl)benzo[d]thiazole

Formaldehyde (2.89 g, 35.66 mmol, 2.67 mL)(37% solution in water) was added to a stirred solution of 5-bromo-2-(3-methyl-4-piperidyl)-1,3-benzothiazole (11.1 g, 35.66 mmol) and acetic acid (4.28 g, 71.33 mmol, 4.08 mL) in MeOH (23.99 mL), resulting mixture stirred at 25° C. for 1 hr, Sodium cyan borohydride (2.24 g, 35.66 mmol) added in 4 portions and stirred at 25° C. for 14 hr. Reaction mixture concentrated, mixed with NaOH (10% in water, 40 mL), extracted with MTBE (2×100 mL), combined organic layer dried over Na₂SO₄, concentrated to give pure 5-bromo-2-(1,3-dimethyl-4-piperidyl)-1,3-benzothiazole (9.5 g, 29.21 mmol, 81.89% yield). LCMS(ESI): [M]⁺ m/z: calcd 325.2; found 326.2; Rt=2.200 min.

### Step 3: Synthesis of 2-(1,3-dimethylpiperidin-4-yl)-5-(4,4,5,5-tetramethyl-1,3,2-dioxaborolan-2-yl)benzo[d]thiazole

Prepared by general procedure scheme S2 step 2. Yield: 1.7 g of crude.

LCMS(ESI): [M]⁺ m/z: calcd 372.2; found 373.2; Rt=2.397 min.

### Step 4: Synthesis of (3S)-tert-butyl 6-(2-(1,3-dimethylpiperidin-4-yl)benzo[d]thiazol-5-yl)-3-methyl-3,4-dihydropyridine-1(2H)-carboxylate

Prepared by general procedure scheme S2 step 3. Yield: 2 g of crude.

LCMS(ESI): [M]⁺ m/z: calcd 441.2; found 442.2; Rt=3.470 min.

### Step 5: Synthesis of 2-(1,3-dimethylpiperidin-4-yl)-5-((S)-5-methyl-3,4,5,6-tetrahydropyridin-2-yl)benzo[d]thiazole

Prepared by general procedure scheme S2 step 4. Yield: 0.8 g of crude.

LCMS(ESI): [M]⁺ m/z: calcd 341.2; found 342.2; Rt=1.249 min.

### Step 6: Synthesis of 2-(1,3-dimethylpiperidin-4-yl)-5-((2R,5S)-5-methylpiperidin-2-yl)benzo[d]thiazole

Sodium cyano borohydride (147.21 mg, 2.34 mmol) was added to a solution of 2-(1,3-dimethyl-4-piperidyl)-5-[(3S)-3-methyl-2,3,4,5-tetrahydropyridin-6-yl]-1,3-benzothiazole (0.8 g, 2.34 mmol) and acetic acid (281.35 mg, 4.69 mmol, 268.21 μL) in MeOH (9.73 mL) and stirred at 25° C. for 12 hr. Reaction mixture was concentrated, diluted with NaOH (20% solution, 5 ml), extracted with DCM (2×20 mL). Combined organic extract dried over Na₂SO₄, solvent evaporated to give crude 2-(1,3-dimethyl-4-piperidyl)-5-[(5S)-5-methyl-2-piperidyl]-1,3-benzothiazole (0.6 g, 1.75 mmol, 74.56% yield). LCMS(ESI): [M]⁺ m/z: calcd 343.2; found 344.2; Rt=1.577 min.

### Step 7: Synthesis of 2,2,2-trifluoroethyl 2-((2R,5S)-2-(2-(1,3-dimethylpiperidin-4-yl)benzo[d]thiazol-5-yl)-5-methylpiperidin-1-yl)-2-oxoacetate

Prepared by general procedure Scheme S3 step 1. Yield: 0.8 g of crude.

LCMS(ESI): [M]⁺ m/z: calcd 497.2; found 498.2; Rt=3.098 min.

### Step 8: Synthesis of 2-((2R,5S)-2-(2-(1,3-dimethylpiperidin-4-yl)benzo[d]thiazol-5-yl)-5-methylpiperidin-1-yl)-2-oxoacetamide

Prepared by general procedure Scheme S3 step 2. Yield: 0.16 g of crude.

LCMS(ESI): [M]⁺ m/z: calcd 414.2; found 415.2; Rt=2.280 min.

### Step 9: Synthesis of N-(4-amino-1-((2-(trimethylsilyl)ethoxy)methyl)-1H-pyrazolo[4,3-c]pyridin-7-yl)-2-((2R,5S)-2-(2-(1,3-dimethylpiperidin-4-yl)benzo[d]thiazol-5-yl)-5-methylpiperidin-1-yl)-2-oxoacetamide

A mixture of 2-oxo-2-[(2R,5S)-2-[2-(1,3-dimethyl-4-piperidyl)-1,3-benzothiazol-5-yl]-5-methyl-1-piperidyl]acetamide (136.00 mg, 328.06 μmol), 7-bromo-1-(2-trimethylsilylethoxymethyl)pyrazolo[4,3-c]pyridin-4-amine (123.88 mg, 360.86 μmol), copper (15.64 mg, 246.04 μmol), copper (I) iodide (31.24 mg, 164.03 μmol, 5.56 μL), cesium carbonate (320.66 mg, 984.17 μmol) and (1R,2R)—N1,N2-dimethylcyclohexane-1,2-diamine (90.99 mg, 639.71 μmol) in dioxane (3.5 mL) was stirred under argon at 95° C. for 48 hr. Reaction mixture cooled down, filtered, filtrated concentrated and submitted to HPLC (column: XBridge C18 100×19 mm, 5 um; mobile phase: 40-65% 0-5 min H₂O/MeCN/0.1% NH₄OH, flow rate: 30 ml/min) to give pure N-[4-amino-1-(2-trimethylsilylethoxymethyl)pyrazolo[4,3-c]pyridin-7-yl]-2-oxo-2-[(2R,5S)-2-[2-(1,3-dimethyl-4-piperidyl)-1,3-benzothiazol-5-yl]-5-methyl-1-piperidyl]acetamide (46 mg, 67.95 μmol, 20.71% yield). LCMS(ESI): [M]⁺ m/z: calcd 676.2; found 677.2; Rt=2.392 min.

### Step 10: Synthesis of N-(4-amino-1H-pyrazolo[4,3-c]pyridin-7-yl)-2-((2R,5S)-2-(2-(1,3-dimethylpiperidin-4-yl)benzo[d]thiazol-5-yl)-5-methylpiperidin-1-yl)-2-oxoacetamide (Compound 31)

Solution of N-[4-amino-1-(2-trimethylsilylethoxymethyl)pyrazolo[4,3-c]pyridin-7-yl]-2-oxo-2-[(2R,5S)-2-[2-(1,3-dimethyl-4-piperidyl)-1,3-benzothiazol-5-yl]-5-methyl-1-piperidyl]acetamide (46 mg, 67.95 μmol) in TFA (774.79 mg, 6.80 mmol, 523.51 μL) was stirred at 25° C. for 2 hr. The crude was purified by HPLC (column: YMC Triart C18 100×20 mm, 5 um; mobile phase 40-90% 0-5 min H₂O/MeOH/0.1% NH₄OH, flow rate: 30 ml/min) to give pure N-(4-amino-1H-pyrazolo[4,3-c]pyridin-7-yl)-2-oxo-2-[(2R,5S)-2-[2-(1,3-dimethyl-4-piperidyl)-1,3-benzothiazol-5-yl]-5-methyl-1-piperidyl]acetamide (30.7 mg, 82.6%).

Compound 31: LCMS(ESI): [M]⁺ m/z: calcd 546.2; found 547.2; Rt=0.949 min.

### Example 35. Synthesis of 2-((2R,5S)-5-methyl-2-(2-(1-methylpiperidin-3-yl)benzo[d]thiazol-5-yl)piperidin-1-yl)-2-oxo-N-(1H-pyrazolo[4,3-c]pyridin-7-yl)acetamide (Compound 3)

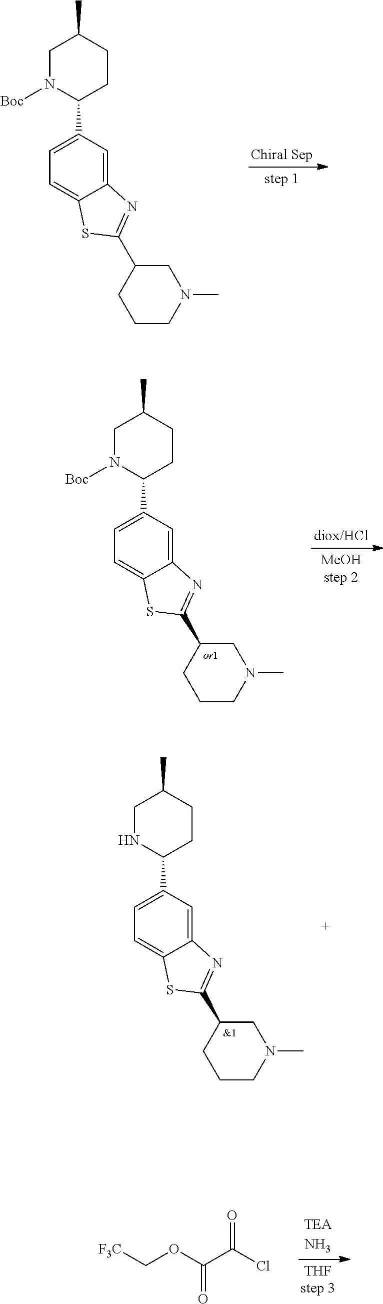 [SMILES: C[C@H]1CC[C@H](c2ccc3sc(C4CCCN(C)C4)nc3c2)N(C(=O)OC(C)(C)C)C1 ; C[C@H]1CC[C@H](c2ccc3sc([C@@H]4CCCN(C)C4)nc3c2)N(C(=O)OC(C)(C)C)C1 ; C[C@H]1CC[C@H](c2ccc3sc([C@@H]4CCCN(C)C4)nc3c2)NC1 ; O=C(Cl)C(=O)OCC(F)(F)F]

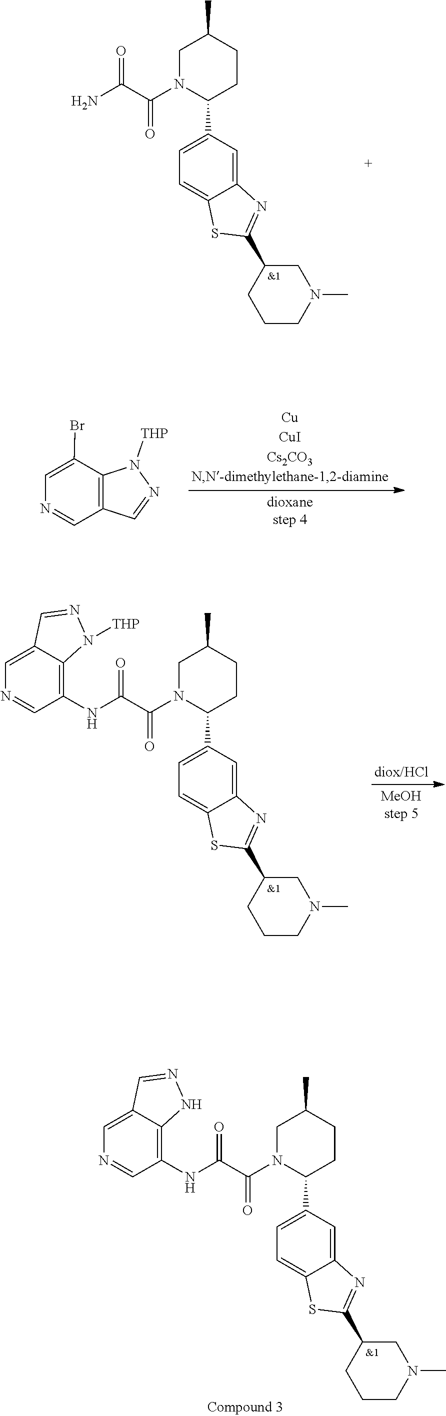

The synthesis of (2R,5S)-tert-butyl 5-methyl-2-(2-(1-methylpiperidin-3-yl)benzo[d]thiazol-5-yl)piperidine-1-carboxylate is given in Intermediate 20.

### Step 1: Chiral Separation

Racemic tert-butyl (2R,5S)-5-methyl-2-[2-(1-methyl-3-piperidyl)-1,3-benzothiazol-5-yl]piperidine-1-carboxylate (0.99 g, 2.30 mmol) was chiral separated (Column: Chiralpak AD-H—III (250*20 mm, 5 mkm), Hexane-IPA-MeOH, 80-10-10, 12 ml/min) to obtain tert-butyl (2R,5S)-5-methyl-2-[2-[(3R)-1-methyl-3-piperidyl]-1,3-benzothiazol-5-yl]piperidine-1-carboxylate (373 mg, 868.21 μmol, 75.35% yield)

Rel Time for this isomer in analytical conditions (column: AD-H, Hexane-IPA-MeOH, 80-10-10, 0.8 ml/min as mobile phase) 8.88 min.

Retention time: 8.88 min LCMS(ESI): [M]⁺ m/z: calcd 429.2; found 430.2; Rt=1.266 min.

### Step 2: Synthesis of 5-((2R,5S)-5-methylpiperidin-2-yl)-2-(rac-(R)-1-methylpiperidin-3-yl)benzo[d]thiazole

A solution of tert-butyl (2R,5S)-5-methyl-2-[2-[(3R)-1-methyl-3-piperidyl]-1,3-benzothiazol-5-yl]piperidine-1-carboxylate (373 mg, 868.21 μmol) in MeOH (3.60 mL) and hydrogen chloride solution 4.0M in dioxane (633.12 mg, 17.36 mmol, 791.40 μL) was stirred at 25° C. for 3 hr. The solvent was evaporated to give 5-[(2R,5S)-5-methyl-2-piperidyl]-2-[rac-(3R)-1-methyl-3-piperidyl]-1,3-benzothiazole (381 mg, 868.11 μmol, 99.99% yield, ³HCl).

The compound was obtained as racemate as a result of complete racemization during HCl deprotection step.

LCMS(ESI): [M]⁺ m/z: calcd 329.2; found 330.2; Rt=0.732 min.

### Step 3: Synthesis of 2-((2R,5S)-5-methyl-2-(2-(rac-(r)-1-methylpiperidin-3-yl)benzo[d]thiazol-5-yl)piperidin-1-yl)-2-oxoacetamide

To a stirred at 0° C. solution of 5-[(2R,5S)-5-methyl-2-piperidyl]-2-[rac-(3R)-1-methyl-3-piperidyl]-1,3-benzothiazole (180 mg, 410.13 μmol, ³HCl) and TEA (249.01 mg, 2.46 mmol, 342.98 μL) in THF (10 mL), 2,2,2-trifluoroethyl 2-chloro-2-oxo-acetate (156.26 mg, 820.26 μmol) was added. The resulting mixture was stirred for 12 hr. Through the solution, Ammonia was bubbled for 10 min at 0° C. The resulting mixture was stirred for 1 hr, the precipitate was filtered off. The solvent was evaporated to give 2-oxo-2-[(2R,5S)-5-methyl-2-[2-[rac-(3R)-1-methyl-3-piperidyl]-1,3-benzothiazol-5-yl]-1-piperidyl]acetamide (160 mg, 399.46 μmol, 97.40% yield). LCMS(ESI): [M]⁺ m/z: calcd 400.2; found 401.2; Rt=0.915 min.

### Step 4: Synthesis of 2-((2R,5S)-5-methyl-2-(2-(rac-(R)-1-methylpiperidin-3-yl)benzo[d]thiazol-5-yl)piperidin-1-yl)-2-oxo-N-(1-(tetrahydro-2H-pyran-2-yl)-1H-pyrazolo[4,3-c]pyridin-7-yl)acetamide

To a mixture of 2-oxo-2-[(2R,5S)-5-methyl-2-[2-[rac-(3R)-1-methyl-3-piperidyl]-1,3-benzothiazol-5-yl]-1-piperidyl]acetamide (160 mg, 399.46 μmol), 7-bromo-1-tetrahydropyran-2-yl-pyrazolo[4,3-c]pyridine (135.24 mg, 479.36 μmol), copper (I) iodide (76.08 mg, 399.46 μmol, 13.54 μL), cesium carbonate (260.31 mg, 798.93 μmol) and rac-(1R,2R)—N1,N2-dimethylcyclohexane-1,2-diamine (85.23 mg, 599.20 μmol) in dioxane, copper (5.08 mg, 79.89 μmol) was added. The resulting mixture was evacuated, refiled with Argon three times, heated at 100° C. for 48 hr and cooled. The inorganic precipitate was filtered and solution was subjected to HPLC (Device (Mobile Phase, Column): SYSTEM 55-100% 0-5 min H₂O/MeOH/0.1% NH₄OH, flow: 30 ml/min (loading pump 4 ml/min MeOH); column: XBridge C18 100×19 mm, 5 um) to give 2-oxo-2-[(2R,5S)-5-methyl-2-[2-[rac-(3R)-1-methyl-3-piperidyl]-1,3-benzothiazol-5-yl]-1-piperidyl]-N-(1-tetrahydropyran-2-ylpyrazolo[4,3-c]pyridin-7-yl)acetamide (47 mg, 78.10 μmol, 19.55% yield. LCMS(ESI): [M]⁺ m/z: calcd 601.2; found 602.2; Rt=2.183 min.

### Step 5: Synthesis of 2-((2R,5S)-5-methyl-2-(2-(1-methylpiperidin-3-yl)benzo[d]thiazol-5-yl)piperidin-1-yl)-2-oxo-N-(1H-pyrazolo[4,3-c]pyridin-7-yl)acetamide (Compound 3)

A solution of 2-oxo-2-[(2R,5S)-5-methyl-2-[2-[rac-(3R)-1-methyl-3-piperidyl]-1,3-benzothiazol-5-yl]-1-piperidyl]-N-(1-tetrahydropyran-2-ylpyrazolo[4,3-c]pyridin-7-yl)acetamide (47 mg, 78.10 μmol) and hydrogen chloride solution 4.0M in dioxane (2 g, 54.85 mmol, 2.50 mL) in MeOH (2 mL) was stirred at 25° C. for 12 hr. The solvent was evaporated and residue purified by HPLC (Device (Mobile Phase, Column): SYSTEM 30-80% 0-5 min H₂O/MeOH/0.1% NH₄OH, flow: 30 ml/min (loading pump 4 ml/min MeOH); column: XBridge C18 100×19 mm, 5 um) to give 2-oxo-N-(1H-pyrazolo[4,3-c]pyridin-7-yl)-2-[(2R,5S)-5-methyl-2-[2-[rac-(3R)-1-methyl-3-piperidyl]-1,3-benzothiazol-5-yl]-1-piperidyl]acetamide (11 mg, 21.25 μmol, 27.21% yield).

Chiral analysis of final compound showed racemic mixture. Racemization took place during THP- and boc-deprotection.

Compound 3: ¹H NMR (600 MHz, DMSO-d₆) δ (ppm) 1.03-1.10 (m, 3H), 1.31-1.45 (m, 1H), 1.52-1.65 (m, 2H), 1.69-1.80 (m, 2H), 1.83-1.99 (m, 1H), 1.99-2.20 (m, 3H), 2.22 (s, 3H), 2.24-2.37 (m, 2H), 2.61-2.69 (m, 2H), 2.86-3.07 (m, 2H), 3.79-4.10 (m, 1H), 5.55-5.83 (m, 1H), 7.37-7.47 (m, 1H), 7.86-7.95 (m, 1H), 8.01-8.11 (m, 1H), 8.28-8.39 (m, 1H), 8.42-8.55 (m, 1H), 8.87-9.00 (m, 1H), 11.01-11.31 (m, 1H), 12.86-13.25 (m, 1H).

LCMS(ESI): [M]⁺ m/z: calcd 517.2; found 518.2; Rt=2.105 min.

### Example 36. Synthesis of 2-((2R,5S)-5-methyl-2-(2-(1-methyl-2-oxopiperidin-4-yl)benzo[d]thiazol-5-yl)piperidin-1-yl)-2-oxo-N-(1H-pyrazolo[3,4-c]pyridin-4-yl)acetamide (Compound 111 and Compound 73)

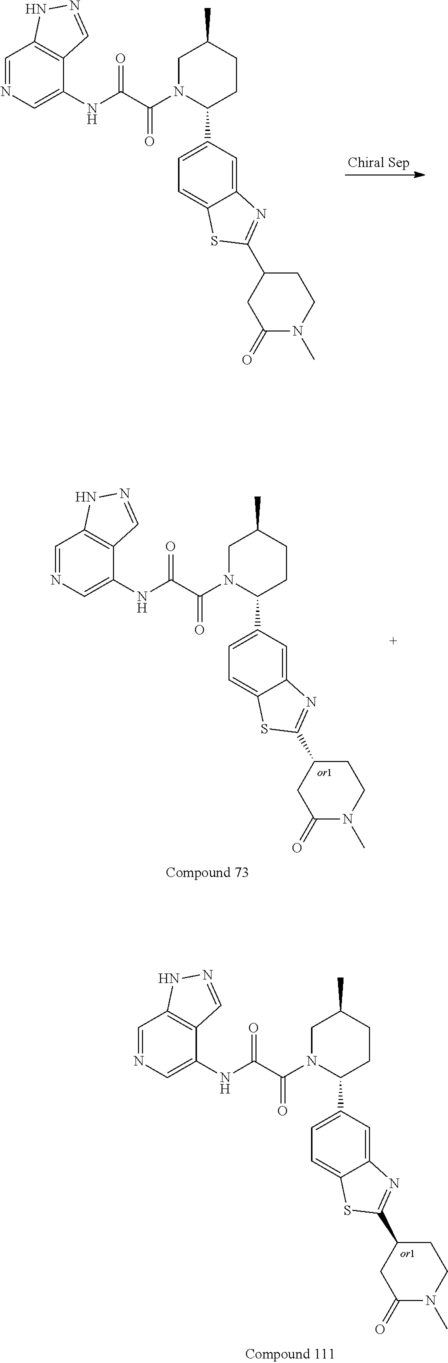 [SMILES: C[C@H]1CC[C@H](c2ccc3sc(C4CCN(C)C(=O)C4)nc3c2)N(C(=O)C(=O)Nc2cncc3[nH]ncc23)C1 ; C[C@H]1CC[C@H](c2ccc3sc([C@@H]4CCN(C)C(=O)C4)nc3c2)N(C(=O)C(=O)Nc2cncc3[nH]ncc23)C1 ; C[C@H]1CC[C@H](c2ccc3sc([C@H]4CCN(C)C(=O)C4)nc3c2)N(C(=O)C(=O)Nc2cncc3[nH]ncc23)C1]

The compounds were prepared starting from 2-((2R,5S)-5-methyl-2-(2-(1-methyl-2-oxopiperidin-4-yl)benzo[d]thiazol-5-yl)piperidin-1-yl)-2-oxoacetamide prepared as described in Example 40, following the general procedures in Scheme S3, starting with step 3A

Chiral Separation Racemic 2-oxo-N-(1H-pyrazolo[3,4-c]pyridin-4-yl)-2-[(2R,5S)-5-methyl-2-[2-(1-methyl-2-oxo-4-piperidyl)-1,3-benzothiazol-5-yl]-1-piperidyl]acetamide (0.0198 g, 37.24 μmol) was chiral separated (Column: Chiralpak IA-III (250-20 mm-5 m); Mobile phase: IPA-MeOH, 50-50 Flow Rate: 12 mL/min) to obtain 2-oxo-N-(1H-pyrazolo[3,4-c]pyridin-4-yl)-2-[(2R,5S)-5-methyl-2-[2-[(4S)-1-methyl-2-oxo-4-piperidyl]-1,3-benzothiazol-5-yl]-1-piperidyl]acetamide (0.01455 g, 27.37 μmol, 73.48% yield) and N-(6-amino-3-pyridyl)-2-[(2S,5R)-5-methyl-2-[2-(trifluoromethyl)-1,3-benzothiazol-5-yl]-1-piperidyl]-2-oxo-acetamide (0.05775 g, 124.60 μmol, 50.22% yield). Rel Time for Compound 111 in analytical conditions (column: IA, IPA-MeOH, 50-50, 0.6 ml/min as mobile phase) 44.85 min and for Compound 73 31.73 min.

Compound 111: Retention time: 44.85 min. ¹H NMR (600 MHz, dmso) δ 1.05-1.09 (m, 3H), 1.34-1.39 (m, 1H), 1.69-1.76 (m, 1H), 1.84-1.98 (m, 2H), 2.06-2.17 (m, 2H), 2.26-2.35 (m, 2H), 2.62-2.67 (m, 1H), 2.70-2.74 (m, 1H), 2.83 (s, 3H), 2.92-3.10 (m, 1H), 3.36-4.10 (m, 3H), 5.22-5.78 (m, 1H), 7.38-7.51 (m, 1H), 7.90-8.01 (m, 1H), 8.02-8.13 (m, 1H), 8.16-8.38 (m, 1H), 8.52-8.67 (m, 1H), 8.74-8.87 (m, 1H), 10.92-11.71 (m, 1H), 13.66 (s, 1H). LCMS(ESI): [M]⁺ m/z: calcd 531.2; found 532.2; Rt=1.083 min.

Compound 73: Retention time: 31.73 min. ¹H NMR (600 MHz, dmso) δ 1.02-1.17 (m, 4H), 1.34-1.40 (m, 1H), 1.68-2.00 (m, 3H), 2.05-2.18 (m, 2H), 2.27-2.35 (m, 2H), 2.61-2.66 (m, 1H), 2.70-2.75 (m, 1H), 2.78-2.92 (m, 4H), 3.40-4.09 (m, 3H), 5.22-5.80 (m, 1H), 7.33-7.53 (m, 1H), 7.87-8.01 (m, 1H), 8.03-8.14 (m, 1H), 8.14-8.38 (m, 1H), 8.48-8.65 (m, 1H), 8.69-8.85 (m, 1H), 11.39 (s, 1H). LCMS(ESI): [M]⁺ m/z: calcd 531.2; found 532.2; Rt=1.084 min.

### Example 37. Synthesis of 2-((2R,5S)-5-methyl-2-(2-(4-methylmorpholin-2-yl)benzo[d]thiazol-5-yl)piperidin-1-yl)-2-oxo-N-(1H-pyrazolo[4,3-c]pyridin-7-yl)acetamide (Compound 39)

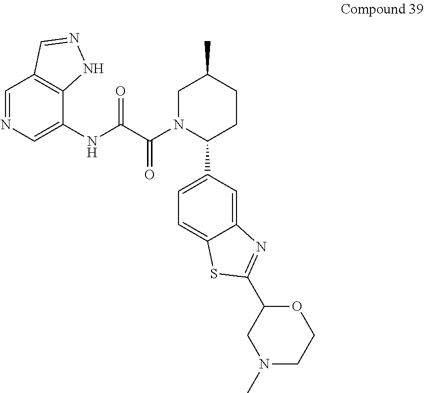 [SMILES: C[C@H]1CC[C@H](c2ccc3sc(C4CN(C)CCO4)nc3c2)N(C(=O)C(=O)Nc2cncc3cn[nH]c23)C1]

The compound was prepared in accordance with Scheme S3, starting with steps 2 and 3A and using 4-methyl-2-(5-((2R,5S)-5-methylpiperidin-2-yl)benzo[d]thiazol-2-yl) morpholine (Intermediate 15).

Compound 39: ¹H NMR (600 MHz, DMSO-d₆) δ (ppm) 1.03-1.10 (m, 3H), 1.31-1.47 (m, 1H), 1.71-1.79 (m, 1H), 1.86-1.98 (m, 1H), 2.07-2.17 (m, 3H), 2.24 (d, 3H), 2.28-2.35 (m, 1H), 2.64-2.91 (m, 2H), 3.13-3.19 (m, 1H), 3.76-4.11 (m, 3H), 4.88-4.96 (m, 1H), 5.59-5.79 (m, 1H), 7.41-7.49 (m, 1H), 7.92-7.98 (m, 1H), 8.06-8.16 (m, 1H), 8.29-8.38 (m, 1H), 8.40-8.54 (m, 1H), 8.88-8.98 (m, 1H), 11.16 (s, 1H), 13.06 (s, 1H).

LCMS(ESI): [M]⁺ m/z: calcd 519.2; found 520.2; Rt=2.291 min.

### Example 38. Synthesis of 2-((2R,5S)-5-methyl-2-(2-(1-(oxetan-3-yl)piperidin-4-yl)benzo[d]thiazol-5-yl)piperidin-1-yl)-2-oxo-N-(1H-pyrazolo[3,4-c]pyridin-4-yl)acetamide (Compound 50)

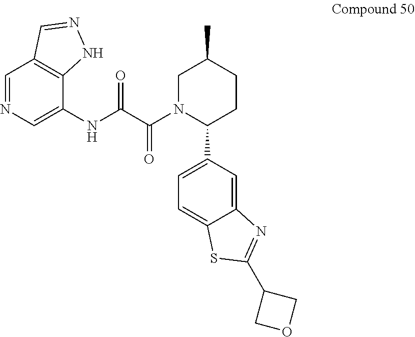 [SMILES: C[C@H]1CC[C@H](c2ccc3sc(C4COC4)nc3c2)N(C(=O)C(=O)Nc2cncc3cn[nH]c23)C1]

The compound was prepared from 2-((2R,5S)-5-methyl-2-(2-(1-(oxetan-3-yl)piperidin-4-yl)benzo[d]thiazol-5-yl)piperidin-1-yl)-2 oxoacetamide (Intermediate 16), in accordance with Scheme S3, starting with step 3A.

Compound 50: ¹H NMR (600 MHZ, DMSO-d₆) δ (ppm) 1.04-1.11 (m, 3H), 1.33-1.42 (m, 1H), 1.68-1.77 (m, 1H), 1.77-1.90 (m, 3H), 1.93-1.98 (m, 2H), 2.07-2.13 (m, 2H), 2.14-2.28 (m, 1H), 2.29-2.36 (m, 1H), 2.73-2.81 (m, 2H), 2.90-2.96 (m, 0.3H), 3.08-3.16 (m, 1H), 3.33-3.38 (m, 0.7H), 3.40-4.11 (m, 2H), 4.40-4.46 (m, 2H), 4.51-4.56 (m, 2H), 5.22-5.80 (m, 1H), 7.36-7.50 (m, 1H), 7.82-7.99 (m, 1H), 7.99-8.12 (m, 1H), 8.16-8.40 (m, 1H), 8.50-8.70 (m, 1H), 8.74-8.91 (m, 1H), 11.22-11.46 (m, 1H), 13.58-13.81 (m, 1H). LCMS(ESI): [M]⁺ m/z: calcd 559.2; found 560.2; Rt=2.384 min.

### Example 39. Synthesis of 2-((2R,5S)-2-(2-((dimethylamino)methyl)benzo[d]thiazol-5-yl)-5-methylpiperidin-1-yl)-2-oxo-N-(1H-pyrazolo[3,4-c]pyridin-4-yl)acetamide (Compound 49)

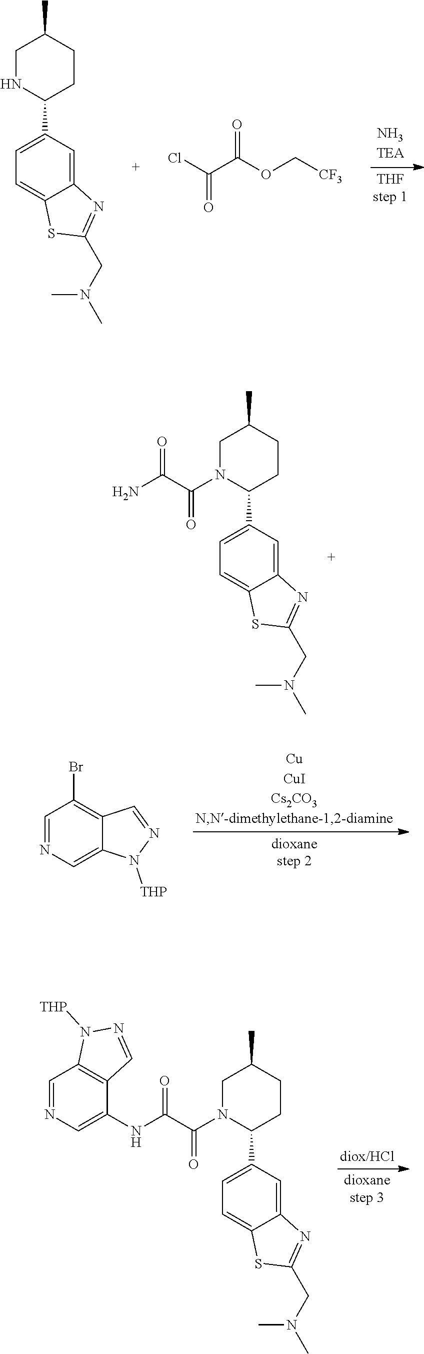

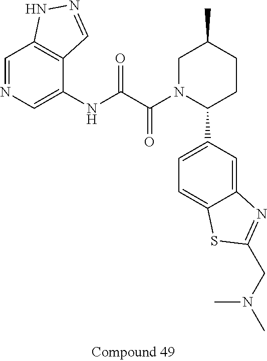 [SMILES: C[C@H]1CC[C@H](c2ccc3sc(CN(C)C)nc3c2)N(C(=O)C(=O)Nc2cncc3[nH]ncc23)C1]

The synthesis of N,N-dimethyl-1-(5-((2R,5S)-5-methylpiperidin-2-yl)benzo[d]thiazol-2-yl) methanamine is given in Intermediate 17.

### Step 1: Synthesis of 2-((2R,5S)-2-(2-((dimethylamino)methyl)benzo[d]thiazol-5-yl)-5-methylpiperidin-1-yl)-2-oxoacetamide

To a solution of N,N-dimethyl-1-[5-[(2R,5S)-5-methyl-2-piperidyl]-1,3-benzothiazol-2-yl]methanamine (0.32 g, 1.11 mmol) and TEA (223.75 mg, 2.21 mmol, 308.19 μL) in THF (5 mL) was added 2,2,2-trifluoroethyl 2-chloro-2-oxo-acetate (210.62 mg, 1.11 mmol) dropwise at 0° C. under argon. The reaction mixture was then stirred for 5 hr at rt, then was added Ammonium (398.90 mg, 22.11 mmol). Resulting solution was stirred at 20° C. for 5 hr. Then, volatiles were removed under reduced pressure, leaving 2-[(2R,5S)-2-[2-[(dimethylamino)methyl]-1,3-benzothiazol-5-yl]-5-methyl-1-piperidyl]-2-oxo-acetamide (0.38 g, 1.05 mmol, 95.35% yield). LCMS(ESI): [M]⁺ m/z: calcd 360.2; found 361.2; Rt=0.712 min.

### Step 2: Synthesis of 2-((2R,5S)-2-(2-((dimethylamino)methyl)benzo[d]thiazol-5-yl)-5-methylpiperidin-1-yl)-2-oxo-N-(1-(tetrahydro-2H-pyran-2-yl)-1H-pyrazolo[3,4-c]pyridin-4-yl)acetamide

Prepared by general procedure Scheme S3 step 3A. Yield: 150 mg of crude.

LCMS(ESI): [M]⁺ m/z: calcd 561.2; found 562.2; Rt=1.050 min.

### Step 3: Synthesis of 2-((2R,5S)-2-(2-((dimethylamino)methyl)benzo[d]thiazol-5-yl)-5-methylpiperidin-1-yl)-2-oxo-N-(1H-pyrazolo[3,4-c]pyridin-4-yl)acetamide

2-[(2R,5S)-2-[2-[(Dimethylamino)methyl]-1,3-benzothiazol-5-yl]-5-methyl-1-piperidyl]-2-oxo-N-(1-tetrahydropyran-2-ylpyrazolo[3,4-c]pyridin-4-yl)acetamide (0.15 g, 267.05 μmol) was dissolved in dioxane (2 mL) and diox/HCl (5 mL) was added thereto. The resulting mixture was stirred overnight. The reaction mixture was concentrated in vacuum and purified by HPLC (2-10 min 10-50% water/MeOH+FA 30 ml/min; loading pump 4 ml/min MeOH+FA; column SunFire 19*100 mm) to obtain 2-[(2R,5S)-2-[2-[(dimethylamino)methyl]-1,3-benzothiazol-5-yl]-5-methyl-1-piperidyl]-2-oxo-N-(1H-pyrazolo[3,4-c]pyridin-4-yl)acetamide (2.10 mg, 4.40 μmol, 1.65% yield).

Compound 49: LCMS(ESI): [M]⁺ m/z: calcd 477.2; found 478.2; Rt=1.676 min.

### Example 40. Synthesis of 2-((2R,5S)-5-methyl-2-(2-(1-methyl-2-oxopiperidin-4-yl)benzo[d]thiazol-5-yl)piperidin-1-yl)-2-oxo-N-(1H-pyrazolo[4,3-c]pyridin-7-yl)acetamide (Compound 98 and Compound 109)

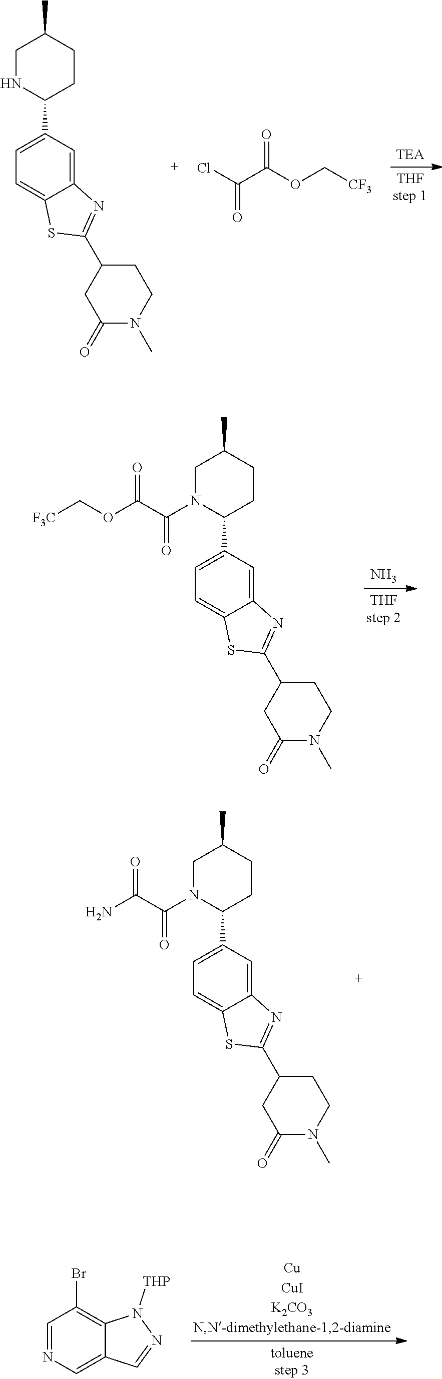

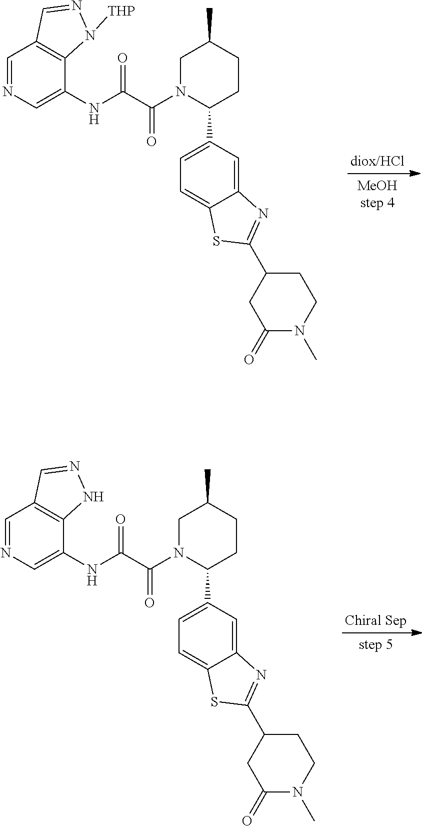

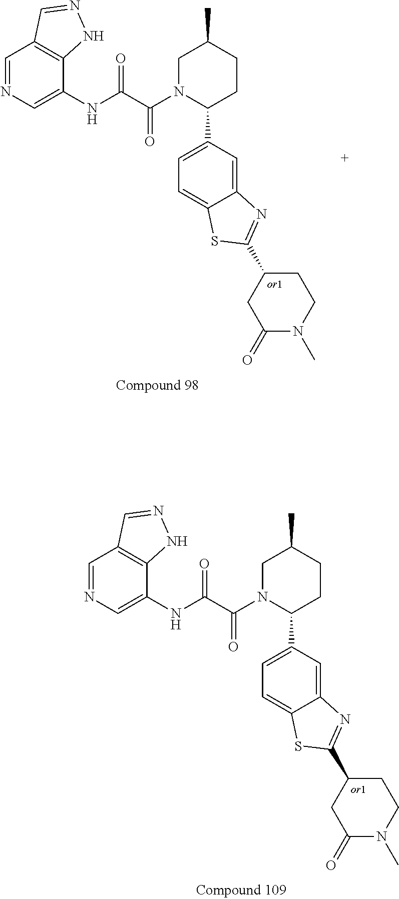 [SMILES: C[C@H]1CC[C@H](c2ccc3sc([C@@H]4CCN(C)C(=O)C4)nc3c2)N(C(=O)C(=O)Nc2cncc3cn[nH]c23)C1 ; C[C@H]1CC[C@H](c2ccc3sc([C@H]4CCN(C)C(=O)C4)nc3c2)N(C(=O)C(=O)Nc2cncc3cn[nH]c23)C1]

The synthesis of 1-methyl-4-(5-((2R,5S)-5-methylpiperidin-2-yl)benzo[d]thiazol-2-yl)piperidin-2-one is given in Intermediate 18.

### Step 1: Synthesis of 2,2,2-trifluoroethyl 2-((2R,5S)-5-methyl-2-(2-(1-methyl-2-oxopiperidin-4-yl)benzo[d]thiazol-5-yl)piperidin-1-yl)-2-oxoacetate

Prepared by general procedure Scheme S3 step 1. Yield: 2 g (92.05%).

LCMS(ESI): [M]⁺ m/z: calcd 497.2; found 498.2; Rt=1.280 min.

### Step 2: Synthesis of 2-((2R,5S)-5-methyl-2-(2-(1-methyl-2-oxopiperidin-4-yl)benzo[d]thiazol-5-yl)piperidin-1-yl)-2-oxoacetamide

Prepared by general procedure Scheme S3 step 2. Yield: 1 g (70.60%).

LCMS(ESI): [M]⁺ m/z: calcd 414.2; found 415.2; Rt=2.464 min.

### Step 3: Synthesis of 2-((2R,5S)-5-methyl-2-(2-(1-methyl-2-oxopiperidin-4-yl)benzo[d]thiazol-5-yl)piperidin-1-yl)-2-oxo-N-(1-(tetrahydro-2H-pyran-2-yl)-1H-pyrazolo[4,3-c]pyridin-7-yl)acetamide

Prepared by general procedure Scheme S3 step 3A. Yield: 85 mg (19.07%).

HPLC conditions: Column: XBridge C18 100*19 mm, 5 microM; 0-1-6 min 40-80% water-MeOH+0.1% NH₄OH, flow: 30 ml/min; (loading pump 4 ml/min MeOH).

LCMS(ESI): [M]⁺ m/z: calcd 615.2; found 616.2; Rt=2.667 min.

### Step 4: Synthesis of 2-((2R,5S)-5-methyl-2-(2-(1-methyl-2-oxopiperidin-4-yl)benzo[d]thiazol-5-yl)piperidin-1-yl)-2-oxo-N-(1H-pyrazolo[4,3-c]pyridin-7-yl)acetamide

To a solution of 2-[(2R,5S)-5-methyl-2-[2-(1-methyl-2-oxo-4-piperidyl)-1,3-benzothiazol-5-yl]-1-piperidyl]-2-oxo-N-(1-tetrahydropyran-2-ylpyrazolo[4,3-c]pyridin-7-yl)acetamide (0.085 g, 138.04 μmol) in MeOH (10 mL) was added hydrogen chloride solution 4.0M in dioxane (800.00 mg, 21.94 mmol, 1 mL) at 21° C. The resulting mixture was left to stir for 6 hr. The resulting mixture was evaporated to dryness and subjected to HPLC (10-10-25% 0-1-6 min H₂O/MeCN/0.1, % NH₄OH flow: 30 ml/min; column: YMX Triart C18 100×20 mm×5 um) to afford 2-[(2R,5S)-5-methyl-2-[2-(1-methyl-2-oxo-4-piperidyl)-1,3-benzothiazol-5-yl]-1-piperidyl]-2-oxo-N-(1H-pyrazolo[4,3-c]pyridin-7-yl)acetamide (0.0172 g, 32.35 μmol, 23.44% yield). LCMS(ESI): [M]⁺ m/z: calcd 531.2; found 532.2; Rt=2.452 min.

### Step 5: Chiral Separation (Compound 98 and Compound 109)

Racemic 2-[(2R,5S)-5-methyl-2-[2-(1-methyl-2-oxo-4-piperidyl)-1,3-benzothiazol-5-yl]-1-piperidyl]-2-oxo-N-(1H-pyrazolo[4,3-c]pyridin-7-yl)acetamide (0.0172 g, 32.35 μmol) was chiral separated (Column: Chiralpak AD-H (250-20 mm-5 m); Mobile phase: IPA-MeOH, 50-50 Flow Rate: 10 mL/min) to obtain 2-[(2R,5S)-5-methyl-2-[2-[rel-(4R)-1-methyl-2-oxo-4-piperidyl]-1,3-benzothiazol-5-yl]-1-piperidyl]-2-oxo-N-(1H-pyrazolo[4,3-c]pyridin-7-yl)acetamide (0.05775 g, 124.60 μmol, 50.22% yield) and 2-[(2R,5S)-5-methyl-2-[2-[rel-(4S)-1-methyl-2-oxo-4-piperidyl]-1,3-benzothiazol-5-yl]-1-piperidyl]-2-oxo-N-(1H-pyrazolo[4,3-c]pyridin-7-yl)acetamide (5.06 mg, 9.52 μmol, 29.42% yield).

Rel Time for Compound 98 in analytical conditions (column: AD-H, IPA-MeOH, 50-50, 0.6 ml/min as mobile phase) 41.05 min and for Compound 109 82.40 min.

Compound 98: Retention time: 41.05 min. LCMS(ESI): [M]⁺ m/z: calcd 531.2; found 532.2; Rt=0.868 min.

Compound 109: Retention time: 82.40 min. LCMS(ESI): [M]⁺ m/z: calcd 531.2; found 532.2; Rt=0.869 min.

### Example 41. Synthesis of 2-((2R,5S)-2-(2-((dimethylamino)methyl)benzo[d]thiazol-5-yl)-5-methylpiperidin-1-yl)-2-oxo-N-(1H-pyrazolo[4,3-c]pyridin-7-yl)acetamide (Compound 29)

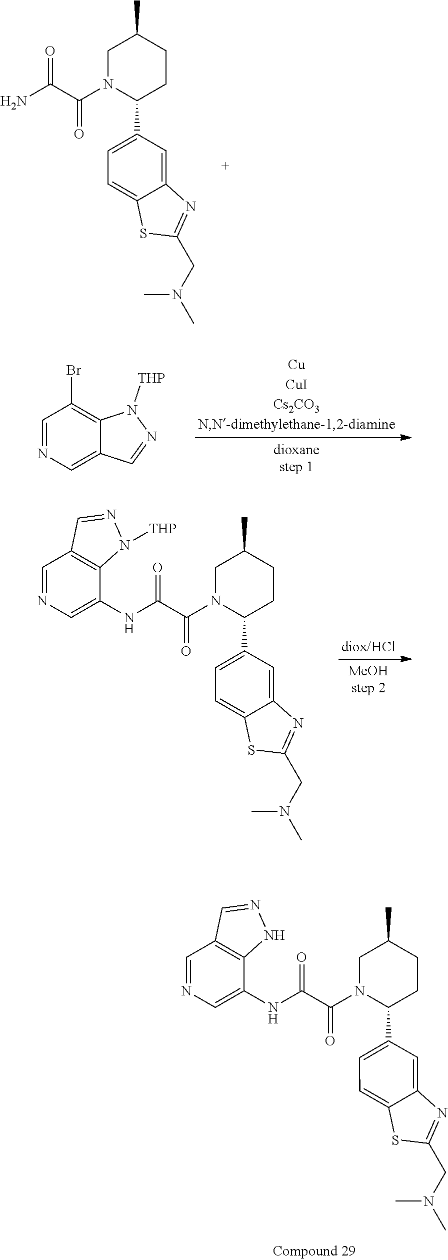

The synthesis of 2-((2R,5S)-2-(2-((dimethylamino)methyl)benzo[d]thiazol-5-yl)-5-methylpiperidin-1-yl)-2-oxoacetamide is given in Example 39.

### Step 1: Synthesis of 2-((2R,5S)-2-(2-((dimethylamino)methyl)benzo[d]thiazol-5-yl)-5-methylpiperidin-1-yl)-2-oxo-N-(1-(tetrahydro-2H-pyran-2-yl)-1H-pyrazolo[4,3-c]pyridin-7-yl)acetamide

Prepared by general procedure Scheme S3 step 3A. Yield: 8.6 mg (5.52%).

HPLC conditions: Column: SunFire C18 100*19 mm, 5 microM; 2-10 min 10-50% water-MeOH+NH₃, flow: 30 ml/min; (loading pump 4 ml/min MeOH+NH₃).

LCMS(ESI): [M]⁺ m/z: calcd 561.2; found 562.2; Rt=0.825 min.

### Step 2: Synthesis of 2-((2R,5S)-2-(2-((dimethylamino)methyl)benzo[d]thiazol-5-yl)-5-methylpiperidin-1-yl)-2-oxo-N-(1H-pyrazolo[4,3-c]pyridin-7-yl)acetamide

2-[(2R,5S)-2-[2-[(Dimethylamino)methyl]-1,3-benzothiazol-5-yl]-5-methyl-1-piperidyl]-2-oxo-N-(1-tetrahydropyran-2-ylpyrazolo[4,3-c]pyridin-7-yl)acetamide (8.6 mg, 15.31 μmol) was dissolved in MeOH (1 mL) and HCl/dioxane (1 mL) was added thereto. The resulting mixture was stirred for 2 hr. The reaction mixture was concentrated in vacuum and purified by HPLC to obtain 2-[(2R,5S)-2-[2-[(dimethylamino)methyl]-1,3-benzothiazol-5-yl]-5-methyl-1-piperidyl]-2-oxo-N-(1H-pyrazolo[4,3-c]pyridin-7-yl)acetamide (0.007 g, 11.93 μmol, 77.89% yield, ³HCl).

Compound 29: ¹H NMR (600 MHz, dmso) δ 0.97-1.12 (m, 3H), 1.37-1.49 (m, 1H), 1.68-1.74 (m, 1H), 1.78-2.06 (m, 2H), 2.14-2.28 (m, 1H), 2.88-2.91 (m, 6H), 3.76-4.14 (m, 2H), 4.81-4.87 (m, 2H), 4.87-5.90 (m, 1H), 7.55-7.69 (m, 1H), 7.98-8.06 (m, 1H), 8.16-8.27 (m, 1H), 8.40-8.76 (m, 1H), 8.78-8.94 (m, 1H), 9.24-9.43 (m, 1H), 10.71-11.14 (m, 1H), 11.59-11.95 (m, 1H), 13.88-14.45 (m, 1H). LCMS(ESI): [M]⁺ m/z: calcd 477.2; found 478.2; Rt=1.875 min.

### Example 42. Synthesis of N-(3H-imidazo[4,5-b]pyridin-6-yl)-2-((2R,5S)-5-methyl-2-(2-(1-methylpiperidin-4-yl)benzo[d]thiazol-5-yl)piperidin-1-yl)-2-oxoacetamide (Compound 69)

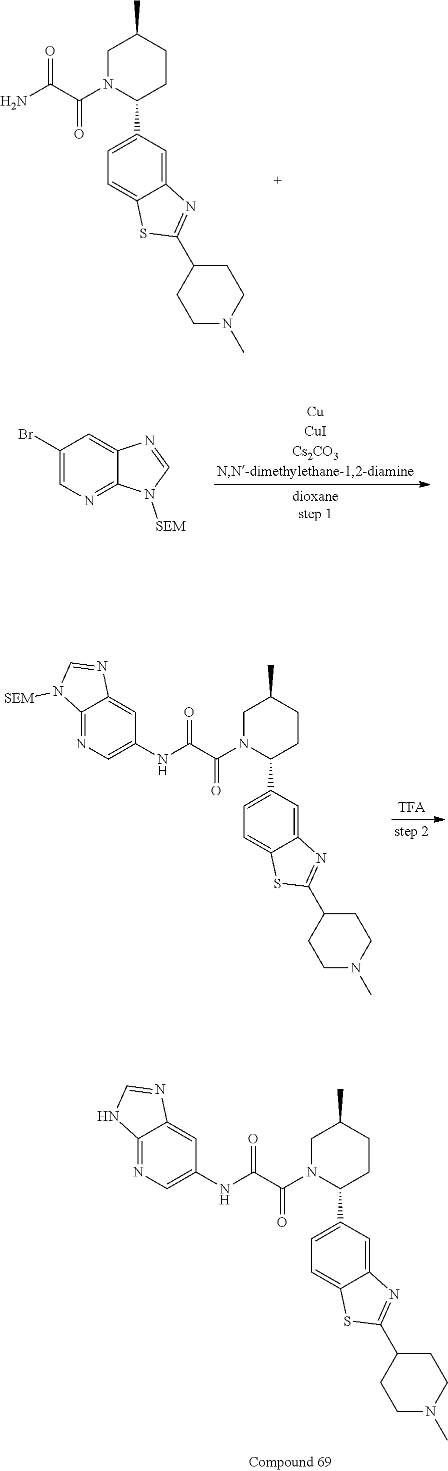 [SMILES: C[C@H]1CC[C@H](c2ccc3sc(C4CCN(C)CC4)nc3c2)N(C(=O)C(N)=O)C1 ; C[Si](C)(C)CCOCn1cnc2cc(Br)cnc21 ; C[C@H]1CC[C@H](c2ccc3sc(C4CCN(C)CC4)nc3c2)N(C(=O)C(=O)Nc2cnc3c(c2)ncn3COCC[Si](C)(C)C)C1 ; C[C@H]1CC[C@H](c2ccc3sc(C4CCN(C)CC4)nc3c2)N(C(=O)C(=O)Nc2cnc3[nH]cnc3c2)C1]

The synthesis of 2-((2R,5S)-5-methyl-2-(2-(1-methylpiperidin-4-yl)benzo[d]thiazol-5-yl)piperidin-1-yl)-2-oxoacetamide is given in Intermediate 19.

The synthesis of f 2-[(6-bromoimidazo[4,5-b]pyridin-3-yl) methoxy]ethyl-trimethyl-silane is given in Intermediate 38

### Step 1: Synthesis of 2-((2R,5S)-5-methyl-2-(2-(1-methylpiperidin-4-yl)benzo[d]thiazol-5-yl)piperidin-1-yl)-2-oxo-N-(3-((2-(trimethylsilyl)ethoxy)methyl)-3H-imidazo[4,5-b]pyridin-6-yl)acetamide

Prepared by general procedure Scheme S3 step 3A. Yield: 113 mg (17.46%).

LCMS(ESI): [M]⁺ m/z: calcd 647.2; found 648.2; Rt=3.243 min.

### Step 2: Synthesis of N-(3H-imidazo[4,5-b]pyridin-6-yl)-2-((2R,5S)-5-methyl-2-(2-(1-methylpiperidin-4-yl)benzo[d]thiazol-5-yl)piperidin-1-yl)-2-oxoacetamide (Compound 69)

2-[(2R,5S)-5-Methyl-2-[2-(1-methyl-4-piperidyl)-1,3-benzothiazol-5-yl]-1-piperidyl]-2-oxo-N-[3-(2-trimethylsilylethoxymethyl) imidazo[4,5-b]pyridin-6-yl]acetamide (110 mg, 169.78 μmol) was diluted with TFA (1.78 g, 15.58 mmol, 1.20 mL). The resulting mixture was stirred at 25° C. for 1 hr. TFA was evaporated in vacuum. The residue was purified by reverse phase HPLC (SYSTEM 20-70% 0-5 min H₂O/MeOH/0.1% NH₄OH, flow: 30 ml/min (loading pump 4 ml/min MeOH) target mass 517.66 column: YMC Triart C18 100×20 mm, 5 um) to give N-(3H-imidazo[4,5-b]pyridin-6-yl)-2-[(2R,5S)-5-methyl-2-[2-(1-methyl-4-piperidyl)-1,3-benzothiazol-5-yl]-1-piperidyl]-2-oxo-acetamide (47 mg, 90.80 μmol, 53.48% yield).

Compound 69: ¹H NMR (500 MHz, DMSO-d₆) δ (ppm) 0.71-1.11 (m, 3H), 1.30-1.43 (m, 1H), 1.69-1.95 (m, 4H), 1.99-2.16 (m, 5H), 2.18 (s, 3H), 2.28-2.36 (m, 1H), 2.81-2.88 (m, 2H), 3.00-3.11 (m, 1H), 3.32-3.37 (m, 1H), 3.46-4.11 (m, 1H), 5.25-5.79 (m, 1H), 7.31-7.48 (m, 1H), 7.84-7.94 (m, 1H), 7.99-8.10 (m, 1H), 8.20-8.43 (m, 2H), 8.43-8.60 (m, 1H), 10.90-11.27 (m, 1H), 12.36-13.38 (m, 1H). LCMS(ESI): [M]⁺ m/z: calcd 517.2; found 518.2; Rt=2.256 min.

### Example 43. Synthesis of N-(8-amino-2,7-naphthyridin-4-yl)-2-((2R,5S)-5-methyl-2-(2-(1,2,2,6,6-pentamethylpiperidin-4-yl)benzo[d]thiazol-5-yl)piperidin-1-yl)-2-oxoacetamide (Compound 46)

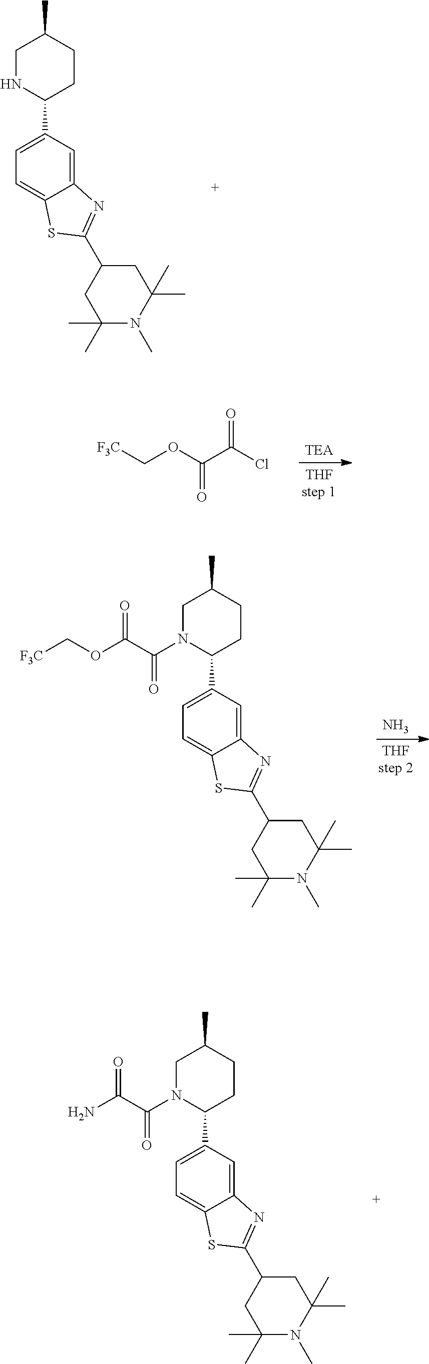

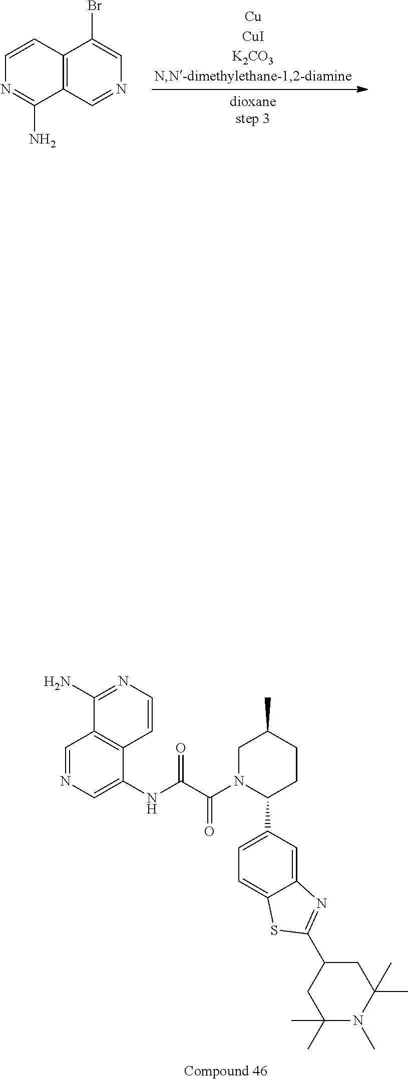 [SMILES: Nc1nccc2c(Br)cncc12 ; C[C@H]1CC[C@H](c2ccc3sc(C4CC(C)(C)N(C)C(C)(C)C4)nc3c2)N(C(=O)C(=O)Nc2cncc3c(N)nccc23)C1]

The synthesis of 5-((2R,5S)-5-methylpiperidin-2-yl)-2-(1,2,2,6,6-pentamethylpiperidin-4-yl)benzo[d]thiazole is given in Intermediate 7

### Step 1: Synthesis of 2,2,2-trifluoroethyl 2-((2R,5S)-5-methyl-2-(2-(1,2,2,6,6-pentamethylpiperidin-4-yl)benzo[d]thiazol-5-yl)piperidin-1-yl)-2-oxoacetate

Prepared by general procedure Scheme S3 step 1. Yield: 1.2 g (61.25%).

LCMS(ESI): [M]⁺ m/z: calcd 539.2; found 540.2; Rt=2.938 min.

### Step 2: Synthesis of 2-((2R,5S)-5-methyl-2-(2-(1,2,2,6,6-pentamethylpiperidin-4-yl)benzo[d]thiazol-5-yl)piperidin-1-yl)-2-oxoacetamide

Prepared by general procedure Scheme S3 step 2. Yield: 1 g (98.48%).

LCMS(ESI): [M]⁺ m/z: calcd 456.2; found 457.2; Rt=2.293 min.

### Step 3: Synthesis of N-(8-amino-2,7-naphthyridin-4-yl)-2-((2R,5S)-5-methyl-2-(2-(1,2,2,6,6-pentamethylpiperidin-4-yl)benzo[d]thiazol-5-yl)piperidin-1-yl)-2-oxoacetamide (Compound 46)

Prepared by general procedure Scheme S3 step 3A. Yield: 5.2 mg (1.58%).

HPLC conditions: Column: YMC Triart C18 100*19 mm, 5 microM; 0-5 min 20-60% water-MeCN+0.1% NH₄OH, flow: 30 ml/min; (loading pump 4 ml/min MeCN).

Compound 46: ¹H NMR (600 MHz, DMSO-d₆) δ (ppm) 1.10 (m, 14H), 1.37 (m, 2H), 1.63 (m, 3H), 1.72 (m, 1H), 1.95 (m, 4H), 2.21 (s, 6H), 4.05 (m, 1H), 5.52 (m, 1H), 6.82 (m, 1H), 7.33 (m, 3H), 7.95 (m, 3H), 8.71 (m, 1H), 9.34 (m, 1H). LCMS(ESI): [M]⁺ m/z: calcd 599.2; found 600.2; Rt=2.387 min.

### Example 44. Synthesis of N-(8-amino-2,7-naphthyridin-4-yl)-2-((2R,5S)-2-(2-((R)-1-(dimethylamino) propan-2-yl)benzo[d]thiazol-5-yl)-5-methylpiperidin-1-yl)-2-oxoacetamide and N-(8-amino-2,7-naphthyridin-4-yl)-2-((2R,5S)-2-(2-((S)-1-(dimethylamino) propan-2-yl)benzo[d]thiazol-5-yl)-5-methylpiperidin-1-yl)-2-oxoacetamide (Compound 99 and Compound 76)

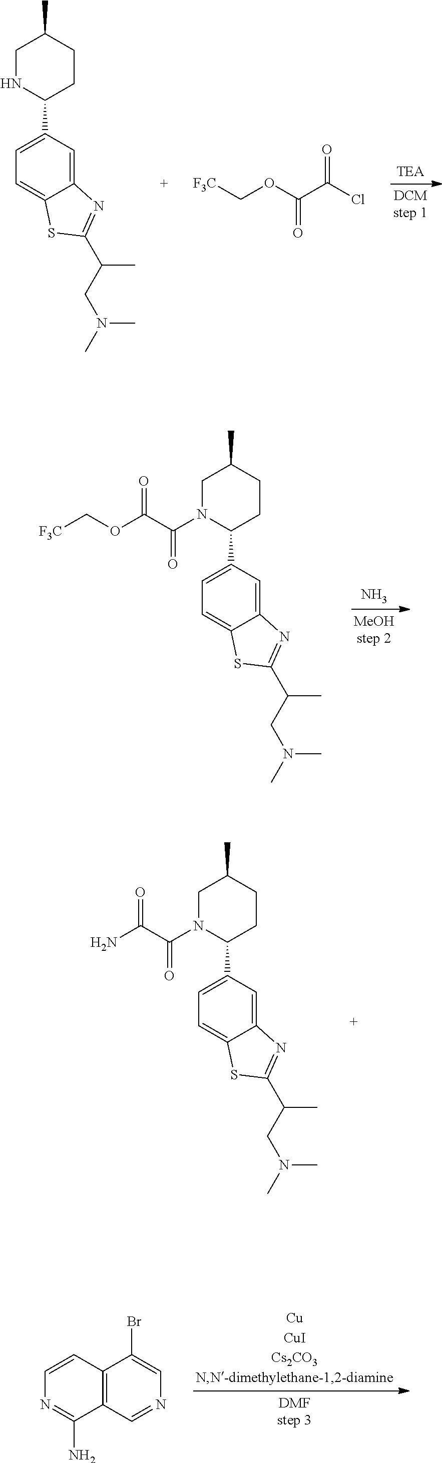

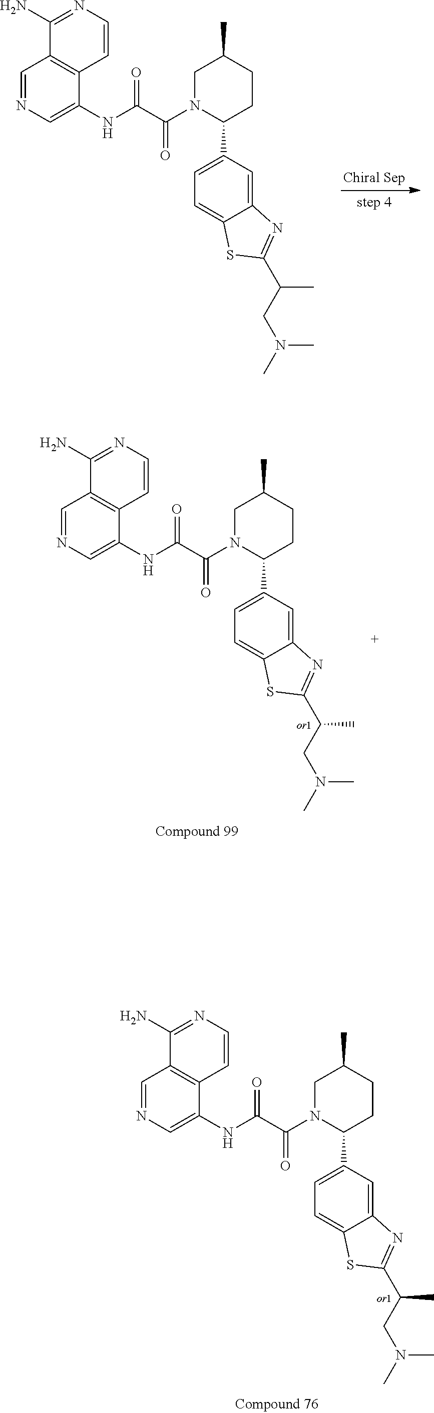 [SMILES: CC(CN(C)C)c1nc2cc([C@H]3CC[C@H](C)CN3C(=O)C(=O)Nc3cncc4c(N)nccc34)ccc2s1 ; C[C@H]1CC[C@H](c2ccc3sc([C@H](C)CN(C)C)nc3c2)N(C(=O)C(=O)Nc2cncc3c(N)nccc23)C1 ; C[C@H]1CC[C@H](c2ccc3sc([C@@H](C)CN(C)C)nc3c2)N(C(=O)C(=O)Nc2cncc3c(N)nccc23)C1]

The Synthesis of N,N-dimethyl-2-(5-((2R,5S)-5-methylpiperidin-2-yl)benzo[d]thiazol-2-yl) propan-1-amine is given in Intermediate 8.

### Step 1: Synthesis of 2,2,2-trifluoroethyl 2-((2R,5S)-2-(2-(1-(dimethylamino) propan-2-5 yl)benzo[d]thiazol-5-yl)-5-methylpiperidin-1-yl)-2-oxoacetate

N,N-Dimethyl-2-[5-[(2R,5S)-5-methyl-2-piperidyl]-1,3-benzothiazol-2-yl]propan-1-amine (0.2 g, 629.94 μmol) and TEA (95.62 mg, 944.91 μmol, 131.70 μL) were mixed together in DCM (10 mL) and the resulting solution was cooled to 5° C. in an ice bath. 2,2,2-Trifluoroethyl 2-chloro-2-oxo-acetate (132.01 mg, 692.93 μmol) was added dropwise to the previous solution and the resulting mixture was allowed to warm to rt and stirred for 4 hr. Upon completion of the reaction, the mixture was washed with brine (2*20 ml), organic layer was dried over Na₂SO₄, filtered and concentrated in vacuum to affording 2,2,2-trifluoroethyl 2-oxo-2-[(2R,5S)-2-[2-[2-(dimethylamino)-1-methyl-ethyl]-1,3-benzothiazol-5-yl]-5-methyl-1-piperidyl]acetate (0.3 g, crude). LCMS(ESI): [M]⁺ m/z: calcd 471.2; found 472.2; Rt=1.020 min.

### Step 2: Synthesis of 2-((2R,5S)-2-(2-(1-(dimethylamino) propan-2-yl)benzo[d]thiazol-5-yl)-5-methylpiperidin-1-yl)-2-oxoacetamide

2,2,2-Trifluoroethyl 2-oxo-2-[(2R,5S)-2-[2-[2-(dimethylamino)-1-methyl-ethyl]-1,3-benzothiazol-5-yl]-5-methyl-1-piperidyl]acetate (0.3 g, 636.22 μmol) was dissolved in NH₃/MeOH (10 mL) and the resulting mixture was stirred for 15 hr at 25° C. Upon completion the resulting mixture was evaporated to dryness to afford crude product. LCMS(ESI): [M]⁺ m/z: calcd 388.2; found 389.2; Rt=0.873 min.

### Step 3: Synthesis of N-(8-amino-2,7-naphthyridin-4-yl)-2-((2R,5S)-2-(2-(1-(dimethylamino) propan-2-yl)benzo[d]thiazol-5-yl)-5-methylpiperidin-1-yl)-2-oxoacetamide

2-Oxo-2-[(2R,5S)-2-[2-[2-(dimethylamino)-1-methyl-ethyl]-1,3-benzothiazol-5-yl]-5-methyl-1-piperidyl]acetamide (0.185 g, 476.16 μmol), 5-bromo-2,7-naphthyridin-1-amine (106.69 mg, 476.16 μmol), Cu (30.26 mg, 476.16 μmol), cesium carbonate (310.28 mg, 952.32 μmol), CuI (90.68 mg, 476.16 μmol, 16.14 μL) and (1S,2S)—N,N′-bis-methyl-1,2-cyclohexane-diamine (101.59 mg, 714.24 μmol, 112.63 μL) were mixed in DMF (3.87 mL) under argon. The resulting mixture was allowed to stir at 100° C. for 15 hr in vial. Upon completion of the reaction, dioxane was evaporated and residue was subjected by HPLC (column: XBridge C18 100×19 mm, 5 um; mobile phase: 10-35% 0-5 min H₂O/MeCN/0.1% NH₄OH, flow rate: 30 ml/min (loading pump 4 ml/min MeCN), affording N-(8-amino-2,7-naphthyridin-4-yl)-2-((2R,5S)-2-(2-(1-(dimethylamino) propan-2-yl)benzo[d]thiazol-5-yl)-5-methylpiperidin-1-yl)-2-oxoacetamide (13.2 mg, 24.85 μmol, 5.2% yield). LCMS(ESI): [M]⁺ m/z: calcd 531.2; found 532.2; Rt=2.186 min.

### Step 4: Chiral Separation (Compound 99 and Compound 76)

Racemic N-(8-amino-2,7-naphthyridin-4-yl)-2-((2R,5S)-2-(2-(1-(dimethylamino) propan-2-yl)benzo[d]thiazol-5-yl)-5-methylpiperidin-1-yl)-2-oxoacetamide (13.2 mg, 24.85 μmol) was chiral separated (Column: Chiralcel OJ-H—I (250*20, 5 mkm), Hexane-MeOH-IPA, 70-15-15, 12 ml/min) to obtain N-(8-amino-2,7-naphthyridin-4-yl)-2-((2R,5S)-2-(2-(rel-(R)-1-(dimethylamino) propan-2-yl)benzo[d]thiazol-5-yl)-5-methylpiperidin-1-yl)-2-oxoacetamide (1.91 mg, 5.9 μmol, 14.94% yield) (RT=18.33 min) and N-(8-amino-2,7-naphthyridin-4-yl)-2-((2R,5S)-2-(2-(rel-(S)-1-(dimethylamino) propan-2-yl)benzo[d]thiazol-5-yl)-5-methylpiperidin-1-yl)-2-oxoacetamide (2.67 mg, 7.17 μmol, 20.7% yield) (RT=25.22 min).

Rel Time for Compound 99 in analytical conditions (column: OJ-H, Hexane-IPA-MeOH, 70-15-15, 0.6 ml/min as mobile phase) 16.50 min and for Compound 76 27.02 min.

Compound 99: Retention time: 16.50 min. LCMS(ESI): [M]⁺ m/z: calcd 531.2; found 532.2; Rt=2.114 min.

Compound 76: Retention time: 27.02 min. LCMS(ESI): [M]⁺ m/z: calcd 531.2; found 532.2; Rt=2.111 min.

### Example 45. The synthesis of N-(8-amino-2,7-naphthyridin-4-yl)-2-((2R,5S)-5-methyl-2-(2-(1-methylpiperidin-3-yl)benzo[d]thiazol-5-yl)piperidin-1-yl)-2-oxoacetamide (Compound 8 and Compound 10)

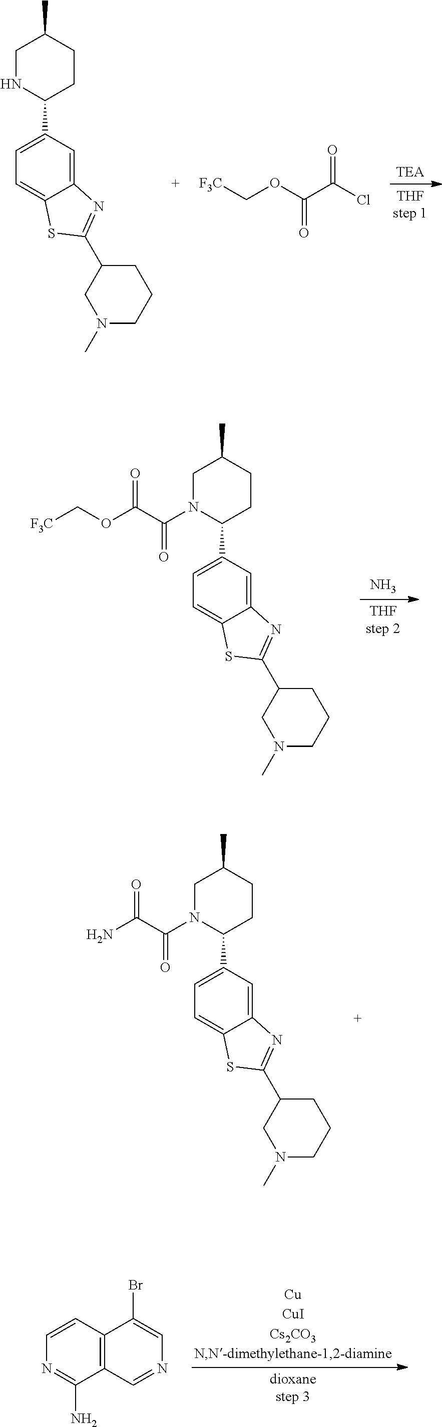

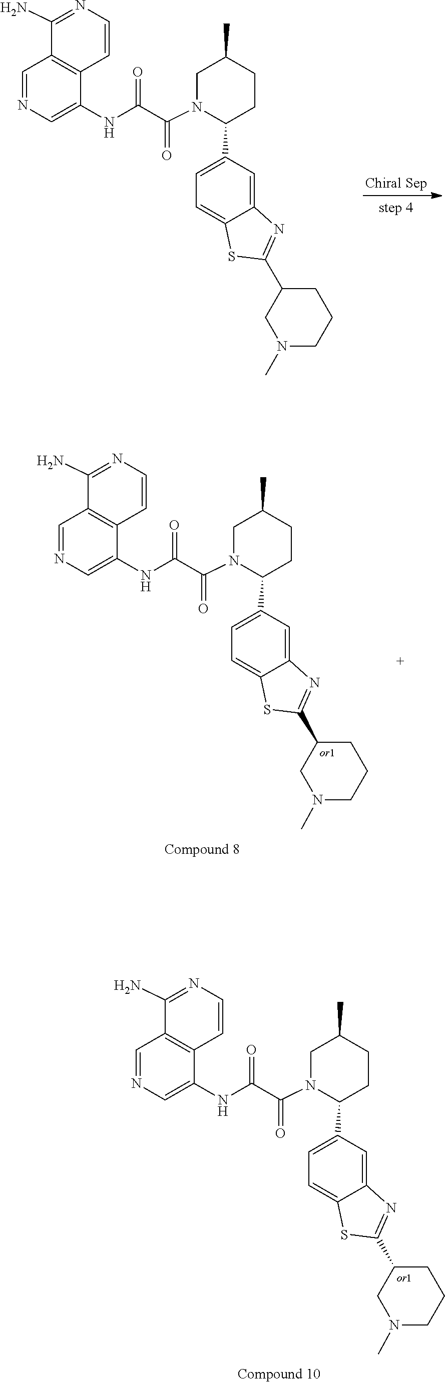 [SMILES: C[C@H]1CC[C@H](c2ccc3sc(C4CCCN(C)C4)nc3c2)N(C(=O)C(=O)Nc2cncc3c(N)nccc23)C1 ; C[C@H]1CC[C@H](c2ccc3sc([C@H]4CCCN(C)C4)nc3c2)N(C(=O)C(=O)Nc2cncc3c(N)nccc23)C1 ; C[C@H]1CC[C@H](c2ccc3sc([C@@H]4CCCN(C)C4)nc3c2)N(C(=O)C(=O)Nc2cncc3c(N)nccc23)C1]

The Synthesis of 5-((2R,5S)-5-methylpiperidin-2-yl)-2-(1-methylpiperidin-3-yl)benzo[d]thiazole is given in Intermediate 20

### Step 1: Synthesis of 2,2,2-trifluoroethyl 2-((2R,5S)-5-methyl-2-(2-(1-methylpiperidin-3-yl)benzo[d]thiazol-5-yl)piperidin-1-yl)-2-oxoacetate

Prepared by general procedure Scheme S3 step 1. Yield: 0.85 g (96.54%).

LCMS(ESI): [M]⁺ m/z: calcd 483.2; found 484.2; Rt=1.107 min.

### Step 2: Synthesis of 2-((2R,5S)-5-methyl-2-(2-(1-methylpiperidin-3-yl)benzo[d]thiazol-5-yl)piperidin-1-yl)-2-oxoacetamide

Prepared by general procedure Scheme S3 step 2. Yield: 0.7 g of crude.

LCMS(ESI): [M]⁺ m/z: calcd 400.2; found 401.2; Rt=0.837 min.

### Step 3: Synthesis of N-(8-amino-2,7-naphthyridin-4-yl)-2-((2R,5S)-5-methyl-2-(2-(1-methylpiperidin-3-yl)benzo[d]thiazol-5-yl)piperidin-1-yl)-2-oxoacetamide

To a mixture of 2-oxo-2-[(2R,5S)-5-methyl-2-[2-(1-methyl-3-piperidyl)-1,3-benzothiazol-5-yl]-1-piperidyl]acetamide (0.4 g, 998.66 μmol), 5-bromo-2,7-naphthyridin-1-amine (223.76 mg, 998.66 μmol), copper (12.69 mg, 199.73 μmol), copper (I) iodide (190.19 mg, 998.66 μmol, 33.84 μL), cesium carbonate (650.77 mg, 2.00 mmol) and (1R,2R)—N1,N2-dimethylcyclohexane-1,2-diamine (213.07 mg, 1.50 mmol), dioxane (6 mL) was added. The resulting mixture was evacuated, refiled with Argon three time, heated at 100° C. for 36 hr and cooled. The inorganic precipitate was filtered and solution was subjected to HPLC (Device (Mobile Phase, Column): SYSTEM 30-75% 0-5 min H₂O/MeOH/0.1% NH₄OH, flow: 30 ml/min (loading pump 4 ml/min MeOH) target mass 544 column: XBridge BEH C18 5 um 130 A) to give 110 mg crude product, which was re-purified (Device (Mobile Phase, Column): SYSTEM 5-35% 0-5 min H₂O/MeCN/0.1% NH₄OH, flow: 30 ml/min (loading pump 4 ml/min MeCN) target mass 544 column: YMC Triart C18 100×20 mm, 5 um) to give N-(8-amino-2,7-naphthyridin-4-yl)-2-oxo-2-[(2R,5S)-5-methyl-2-[2-(1-methyl-3-piperidyl)-1,3-benzothiazol-5-yl]-1-piperidyl]acetamide (60 mg, 110.36 μmol, 11.05% yield). LCMS(ESI): [M]⁺ m/z: calcd 543.2; found 544.2; Rt=2.042 min.

### Step 4: Chiral Separation (Compound 8 and Compound 10)

Racemic N-(8-amino-2,7-naphthyridin-4-yl)-2-oxo-2-[(2R,5S)-5-methyl-2-[2-(1-methyl-3-piperidyl)-1,3-benzothiazol-5-yl]-1-piperidyl]acetamide (60 mg, 110.36 μmol) was chiral separated (Column: Chiralpak IA (250*20 mm, 5 mkm); Mobile phase: IPA-MeOH, 50-50. Flow Rate: 12 mL/min) to obtain N-(8-amino-2,7-naphthyridin-4-yl)-2-oxo-2-[(2R,5S)-5-methyl-2-[2-[rel-(3S)-1-methyl-3-piperidyl]-1,3-benzothiazol-5-yl]-1-piperidyl]acetamide (19 mg, 34.95 μmol, 63.33% yield) and N-(8-amino-2,7-naphthyridin-4-yl)-2-oxo-2-[(2R,5S)-5-methyl-2-[2-[rel-(3R)-1-methyl-3-piperidyl]-1,3-benzothiazol-5-yl]-1-piperidyl]acetamide (17 mg, 31.27 μmol, 56.67% yield). RT (isomer A)=52.97 min; RT (isomer B)=77.25 min.

Rel Time for Compound 8 in analytical conditions (column: IA, IPA-MeOH, 50-50, 0.6 ml/min as mobile phase) 73.45 min and for Compound 10 54.36 min.

Compound 8: Retention time: 73.45 min. ¹H NMR (DMSO-d6, 600 MHz): δ (ppm) 1.09 (m, 3H), 1.37 (m, 1H), 1.61 (m, 2H), 1.72 (m, 2H), 1.92 (m, 1H), 2.04 (m, 2H), 2.13 (m, 1H), 2.22 (s, 3H), 2.32 (m, 1H), 2.65 (m, 1H), 3.02 (m, 2H), 3.37 (m, 2H), 4.09 (m, 1H), 5.52 (m, 1H), 6.85 (m, 1H), 7.36 (m, 3H), 7.86 (m, 1H), 8.04 (m, 2H), 8.69 (m, 1H), 9.38 (m, 1H), 10.91 (m, 1H). LCMS(ESI): [M]⁺ m/z: calcd 543.2; found 544.2; Rt=1.776 min.

Compound 10: Retention time: 54.36 min. ¹H NMR (DMSO-d6, 600 MHz): δ (ppm) 1.09 (m, 3H), 1.37 (m, 1H), 1.61 (m, 2H), 1.72 (m, 2H), 1.92 (m, 1H), 2.04 (s, 2H), 2.13 (m, 1H), 2.22 (s, 4H), 2.32 (m, 2H), 2.65 (m, 1H), 3.02 (m, 2H), 4.09 (m, 1H), 5.52 (m, 1H), 6.84 (m, 1H), 7.36 (m, 3H), 7.86 (m, 1H), 8.04 (m, 2H), 8.69 (m, 1H), 9.38 (m, 1H), 10.91 (m, 1H).

LCMS(ESI): [M]⁺ m/z: calcd 543.2; found 544.2; Rt=0.764 min.

### Example 46. Synthesis of 2-((2R,5S)-2-(2-(3-(dimethylamino) propyl)benzo[d]thiazol-5-yl)-5-methylpiperidin-1-yl)-2-oxo-N-(1H-pyrazolo[4,3-c]pyridin-7-yl)acetamide (Compound 72)

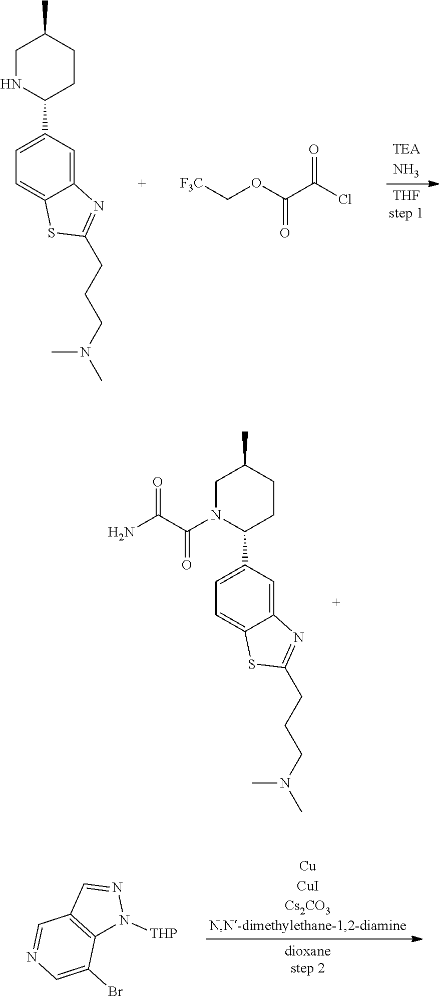

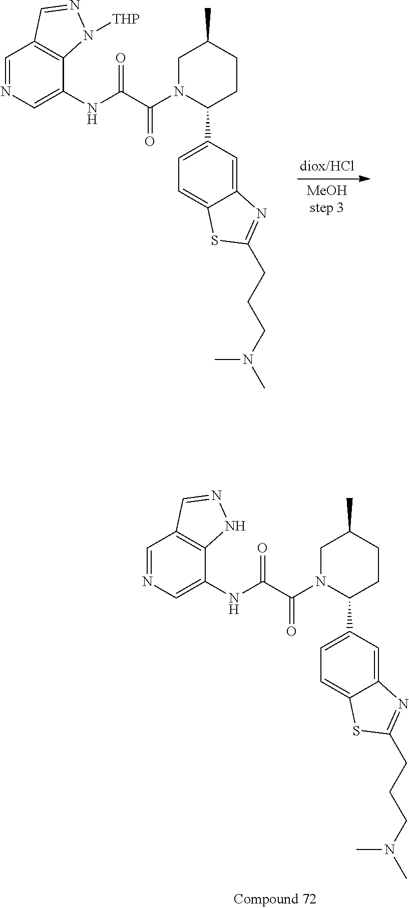

The Synthesis of N,N-dimethyl-3-(5-((2R,5S)-5-methylpiperidin-2-yl)benzo[d]thiazol-2-yl) propan-1-amine is given in Intermediate 21.

### Step 1: Synthesis of 2-((2R,5S)-2-(2-(3-(dimethylamino) propyl)benzo[d]thiazol-5-yl)-5-methylpiperidin-1-yl)-2-oxoacetamide

2,2,2-Trifluoroethyl 2-chloro-2-oxo-acetate (546.03 mg, 2.87 mmol) was slowly added to a stirred solution of N,N-dimethyl-3-[5-[(2R,5S)-5-methyl-2-piperidyl]-1,3-benzothiazol-2-yl]propan-1-amine (700 mg, 2.20 mmol) and TEA (557.76 mg, 5.51 mmol, 768.26 μL) in dry THF (30 mL) at 25° C. The resulting mixture was stirred at 25° C. for 1 hr, then gaseous ammonia was bubbled through the reaction mixture at 25° C. for 0.5-1 hr. The resulting ammonium chloride precipitate was filtered and discarded, the filtrate was concentrated in vacuum to afford crude 2-oxo-2-[(2R,5S)-2-[2-[3-(dimethylamino) propyl]-1,3-benzothiazol-5-yl]-5-methyl-1-piperidyl]acetamide (850 mg, 2.19 mmol, 99.23% yield) as light-brown gum, which was used directly in the next step. LCMS(ESI): [M]⁺ m/z: calcd 388.2; found 389.2; Rt=2.089 min.

### Step 2: Synthesis of 2-((2R,5S)-2-(2-(3-(dimethylamino) propyl)benzo[d]thiazol-5-yl)-5-methylpiperidin-1-yl)-2-oxo-N-(1-(tetrahydro-2H-pyran-2-yl)-1H-pyrazolo[4,3-c]pyridin-7-yl)acetamide

Prepared by general procedure Scheme S3 step 3A. Yield: 140 mg (36.89%).

HPLC conditions: Column: XBridge C18 100*19 mm, 5 microM; 0-5 min 40-90% water-MeOH+0.1% NH₄OH, flow: 30 ml/min; (loading pump 4 ml/min MeOH).

LCMS(ESI): [M]⁺ m/z: calcd 589.2; found 590.2; Rt=1.806 min.

### Step 3: Synthesis of 2-((2R,5S)-2-(2-(3-(dimethylamino) propyl)benzo[d]thiazol-5-yl)-5-methylpiperidin-1-yl)-2-oxo-N-(1H-pyrazolo[4,3-c]pyridin-7-yl)acetamide (Compound 72)

Hydrogen chloride, 4M in dioxane, 99% (787.50 mg, 3.00 mmol, 0.75 mL, 13.9% purity) was added in one portion to a stirred solution of 2-oxo-2-[(2R,5S)-2-[2-[3-(dimethylamino) propyl]-1,3-benzothiazol-5-yl]-5-methyl-1-piperidyl]-N-(1-tetrahydropyran-2-ylpyrazolo[4,3-c]pyridin-7-yl)acetamide (140 mg, 237.39 μmol) in MeOH (3 mL) at 25° C. The resulting solution was stirred at 25° C. for 5 hr, and then evaporated to dryness in vacuum. The resulting light-yellow solid was submitted to reverse phase HPLC (column: YMC Triart C18 100×20 mm, 5 um; mobile phase: 40-70% 0-5 min H₂O/MeOH/0.1% NH₄OH, flow rate: 30 ml/min (loading pump 4 ml/min MeOH)) to afford Compound 72 2-oxo-N-(1H-pyrazolo[4,3-c]pyridin-7-yl)-2-[(2R,5S)-2-[2-[3-(dimethylamino) propyl]-1,3-benzothiazol-5-yl]-5-methyl-1-piperidyl]acetamide (73 mg, 144.37 μmol, 60.82% yield) as light-yellow solid.

Compound 72: ¹H NMR (600 MHz, DMSO-d₆) δ (ppm) 1.03-1.09 (m, 3H), 1.33-1.43 (m, 1H), 1.70-1.80 (m, 1H), 1.88-1.97 (m, 3H), 2.11-2.16 (m, 7H), 2.25-2.34 (m, 3H), 2.87-3.12 (m, 3H), 3.76-4.13 (m, 1H), 5.52-5.81 (m, 1H), 7.36-7.45 (m, 1H), 7.88-7.94 (m, 1H), 7.99-8.09 (m, 1H), 8.29-8.37 (m, 1H), 8.42-8.55 (m, 1H), 8.88-8.98 (m, 1H), 11.03-11.29 (m, 1H), 12.99-13.25 (m, 1H). LCMS(ESI): [M]⁺ m/z: calcd 505.2; found 506.2; Rt=2.083 min.

### Example 47. Synthesis of N-(4-methyl-1H-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (Compound 9)

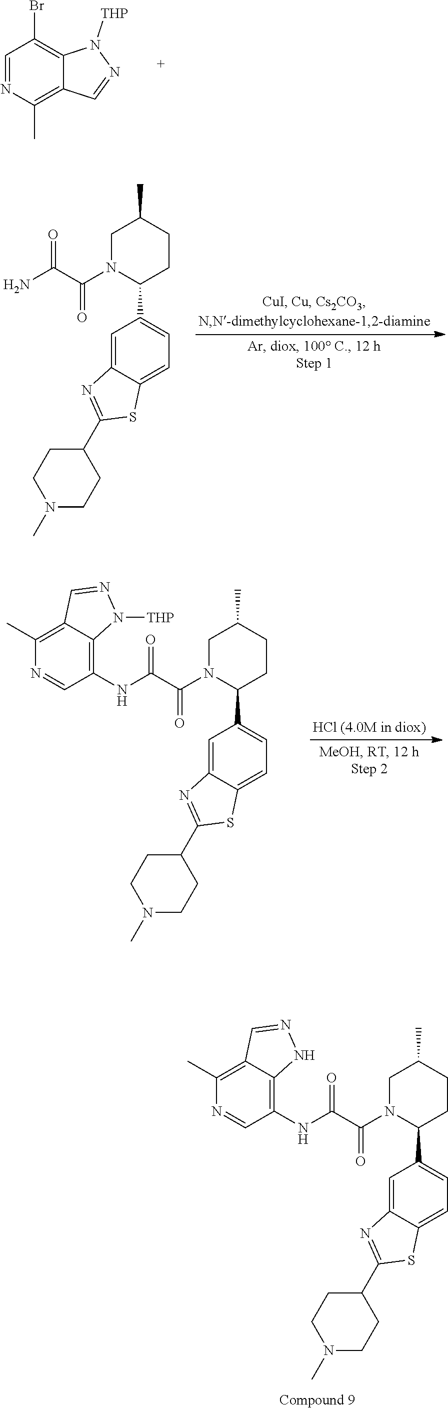

The synthesis of 2-((2R,5S)-5-methyl-2-(2-(1-methylpiperidin-4-yl)benzo[d]thiazol-5-yl)piperidin-1-yl)-2-oxoacetamide is given in Intermediate 19.

The synthesis of 7-bromo-4-methyl-1-tetrahydropyran-2-yl-pyrazolo[4,3-c]pyridine is given in Intermediate 39.

### Step 1: The synthesis of N-(4-methyl-1-tetrahydropyran-2-yl-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide

Prepared by general procedure Scheme S3 step 3A. Yield: 8.1 mg (10.31%).

HPLC conditions: 2-10 min 50-75% water NeCN+FA 30 mL/min; loading pump 4 mL/min ACN; column SunFire 19*100 mm.

LCMS(ESI): [M+2]⁺ m/z: calcd 617.2; found 617.2; Rt=0.943 min.

### Step 2: The synthesis of N-(4-methyl-1H-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (Compound 9)

A mixture of N-(4-methyl-1-tetrahydropyran-2-yl-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (8.1 mg, 13.15 μmol) was stirred in Dioxane/HCl (0.5 mL) and Methanol (0.5 mL) at 35° C. for 12 hr. Solvents were evaporated to obtain N-(4-methyl-1H-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (7 mg, crude, 2HCl).

LCMS(ESI): [M+1]⁺ m/z: calcd 532.2; found 532.2; Rt=2.108 min.

### Example 48. Synthesis of N-(1-amino-2,7-naphthyridin-4-yl)-2-((2R,5S)-5-methyl-2-(2-(1-methylpiperidin-4-yl)benzo[d]thiazol-5-yl)piperidin-1-yl)-2-oxoacetamide (Compound 11)

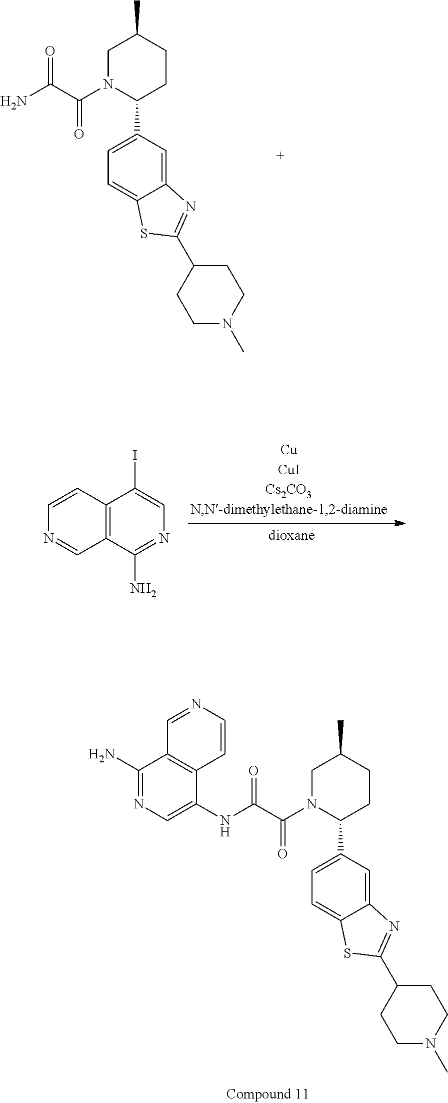 [SMILES: C[C@H]1CC[C@H](c2ccc3sc(C4CCN(C)CC4)nc3c2)N(C(=O)C(N)=O)C1 ; Nc1ncc(I)c2ccncc12 ; C[C@H]1CC[C@H](c2ccc3sc(C4CCN(C)CC4)nc3c2)N(C(=O)C(=O)Nc2cnc(N)c3cnccc23)C1]

The synthesis of 2-((2R,5S)-5-methyl-2-(2-(1-methylpiperidin-4-yl)benzo[d]thiazol-5-yl)piperidin-1-yl)-2-oxoacetamide is given in Intermediate 19.

Synthesis of 4-iodo-2,7-naphthyridin-1-amine is given in Intermediate 40.

Two separate lots were prepared analogically by general procedure Scheme S3 step 3A. Yield: 6.3 mg (3.64%).

HPLC conditions: Column: XBridge C18 100*19 mm, 5 microM; 0-5 min 25-75% water-MeOH+0.1% NH₄OH; (loading pump 4 ml/min MeCN).

Product was purified by chiral HPLC (Chiralcel IC—III (250*20 mm, 5 mkm), IPA-MeOH, 50-50, 10 ml/min).

Compound 11: Retention time: 37.98 min. ¹H NMR (600 MHZ, DMSO-d₆) δ (ppm) 1.06-1.12 (m, 3H), 1.36-1.41 (m, 1H), 1.72-1.79 (m, 1H), 1.79-2.01 (m, 4H), 2.09-2.16 (m, 3H), 2.17-2.29 (m, 2H), 2.29-2.34 (m, 2H), 2.89-3.18 (m, 4H), 3.52-4.11 (m, 2H), 5.25-5.80 (m, 1H), 7.14-7.41 (m, 2H), 7.42-7.63 (m, 2H), 7.89-7.96 (m, 1H), 7.96-8.05 (m, 1H), 8.07-8.11 (m, 1H), 8.35-8.69 (m, 1H), 9.45-9.59 (m, 1H), 10.41-10.65 (m, 1H). LCMS(ESI): [M+1]⁺ m/z: calcd 543.2; found 544.2; Rt=1.914 min.

### Example 49. Synthesis of N-(4-amino-1H-pyrazolo[4,3-c]pyridin-7-yl)-2-((2R,5S)-5-methyl-2-(2-(1-methylpiperidin-4-yl) quinolin-7-yl)piperidin-1-yl)-2-oxoacetamide (Compound 65)

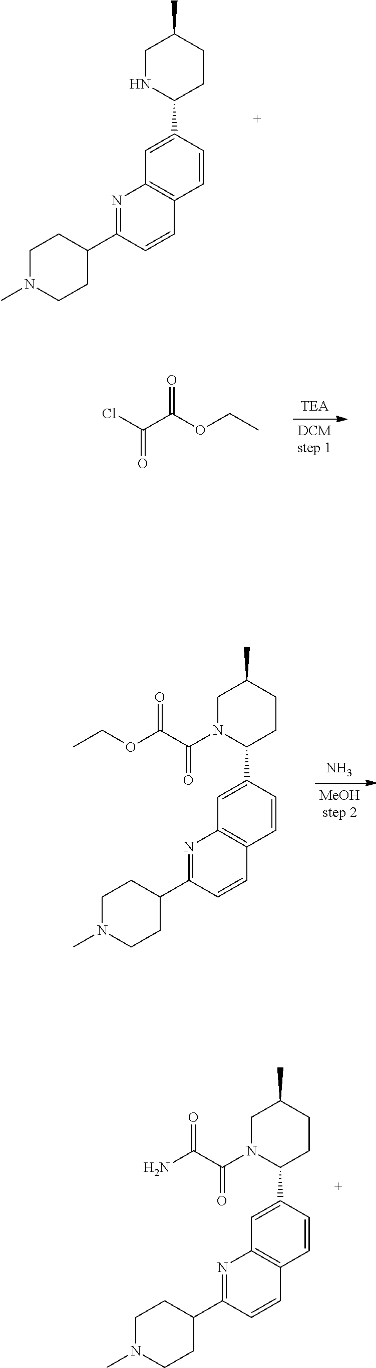

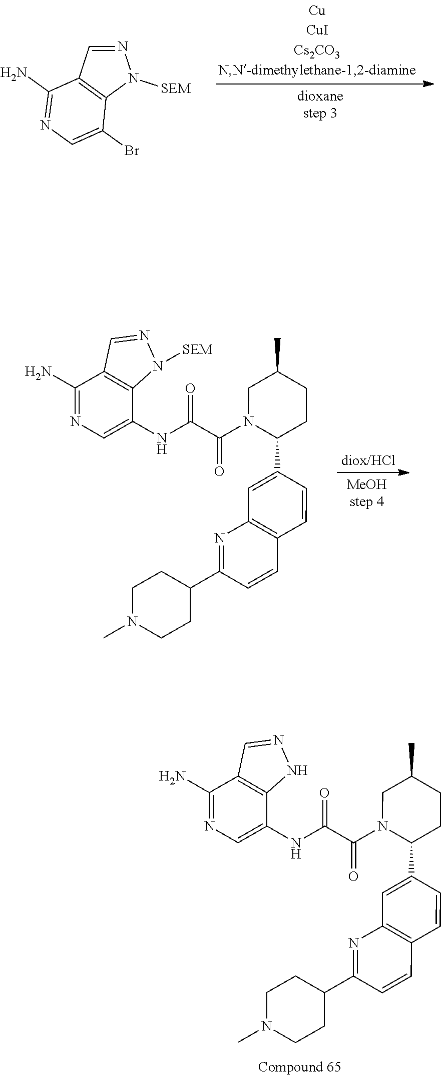 [SMILES: C[Si](C)(C)CCOCn1ncc2c(N)ncc(Br)c21 ; C[C@H]1CC[C@H](c2ccc3ccc(C4CCN(C)CC4)nc3c2)N(C(=O)C(=O)Nc2cnc(N)c3cnn(COCC[Si](C)(C)C)c23)C1 ; C[C@H]1CC[C@H](c2ccc3ccc(C4CCN(C)CC4)nc3c2)N(C(=O)C(=O)Nc2cnc(N)c3cn[nH]c23)C1]

The synthesis of 2-(1-methyl-4-piperidyl)-7-[(2R,5S)-5-methyl-2-piperidyl]quinoline is given in Intermediate 22

### Step 1: Synthesis of ethyl 2-((2R,5S)-5-methyl-2-(2-(1-methylpiperidin-4-yl) quinolin-7-yl)piperidin-1-yl)-2-oxoacetate

2-(1-Methyl-4-piperidyl)-7-[(2R,5S)-5-methyl-2-piperidyl]quinoline (200 mg, 432.80 μmol) and TEA (87.59 mg, 865.60 μmol, 120.65 μL) was dissolved in DCM (15 mL), cooled with ice-water bath and then ethyl 2-chloro-2-oxo-acetate (76.82 mg, 562.64 μmol, 62.97 μL) was added dropwise. Solution was stirred overnight at 25° C. Reaction mixture was washed with aqueous solution of NaHCO₃, dried over sodium sulphate and evaporated to give ethyl 2-oxo-2-[(2R,5S)-5-methyl-2-[2-(1-methyl-4-piperidyl)-7-quinolyl]-1-piperidyl]acetate (240 mg, crude). LCMS(ESI): [M]⁺ m/z: calcd 423.2; found 424.2; Rt=1.060 min.

### Step 2: Synthesis of 2-((2R,5S)-5-methyl-2-(2-(1-methylpiperidin-4-yl) quinolin-7-yl)piperidin-1-yl)-2-oxoacetamide

Ethyl 2-oxo-2-[(2R,5S)-5-methyl-2-[2-(1-methyl-4-piperidyl)-7-quinolyl]-1-piperidyl]acetate (240 mg, 481.65 μmol) was dissolved in NH₃/MeOH (20 mL) solution (10% of NH₃ by weight) and stirred overnight. The RM was concentrated in vacuum, then dissolved in DCM (30 ml) and washed with water. Organic phase was dried over Na₂SO₄ and concentrated in vacuum to give 2-oxo-2-[(2R,5S)-5-methyl-2-[2-(1-methyl-4-piperidyl)-7-quinolyl]-1-piperidyl]acetamide (150 mg, crude). LCMS(ESI): [M]⁺ m/z: calcd 394.2; found 395.2; Rt=0.734 min.

### Step 3: Synthesis of N-(4-amino-1-((2-(trimethylsilyl)ethoxy)methyl)-1H-pyrazolo[4,3-c]pyridin-7-yl)-2-((2R,5S)-5-methyl-2-(2-(1-methylpiperidin-4-yl) quinolin-7-yl)piperidin-1-yl)-2-oxoacetamide

2-Oxo-2-[(2R,5S)-5-methyl-2-[2-(1-methyl-4-piperidyl)-7-quinolyl]-1-piperidyl]acetamide (120 mg, 255.51 μmol), 7-bromo-1-(2-trimethylsilylethoxymethyl)pyrazolo[4,3-c]pyridin-4-amine (105.26 mg, 306.61 μmol), Cu (16.24 mg, 255.51 μmol), cesium carbonate (166.50 mg, 511.02 μmol), copper (I) iodide (48.66 mg, 255.51 μmol, 8.66 μL) and N,N′-dimethylethane-diamine (54.52 mg, 383.26 μmol, 60.44 μL) were mixed in dioxane (5.00 mL) under argon. The resulting mixture was allowed to stir at 100° C. for 12 hr in vial. RM was filtered, concentrated in vacuum, and subjected to HPLC (2-10 min 10-40% MeCN+NH₃ 30 ml/min (loading pump 4 m MeCN) column: SunFire 100*19 mm, 5 microM). N-[4-Amino-1-(2-trimethylsilylethoxymethyl)pyrazolo[4,3-c]pyridin-7-yl]-2-oxo-2-[(2R,5S)-5-methyl-2-[2-(1-methyl-4-piperidyl)-7-quinolyl]-1-piperidyl]acetamide (48.5 mg, 73.83 μmol, 28.90% yield) was obtained. LCMS(ESI): [M]⁺ m/z: calcd 656.2; found 657.2; Rt=1.163 min.

### Step 4: Synthesis of N-(4-amino-1H-pyrazolo[4,3-c]pyridin-7-yl)-2-((2R,5S)-5-methyl-2-(2-(1-methylpiperidin-4-yl) quinolin-7-yl)piperidin-1-yl)-2-oxoacetamide (Compound 65)

N-[4-Amino-1-(2-trimethylsilylethoxymethyl)pyrazolo[4,3-c]pyridin-7-yl]-2-oxo-2-[(2R,5S)-5-methyl-2-[2-(1-methyl-4-piperidyl)-7-quinolyl]-1-piperidyl]acetamide (47.5 mg, 60.02 μmol) was dissolved in MeOH (1 mL), next 3 ml of HCl/diox (60.02 μmol, 3 mL)(10% of HCl by weight) was added and stirred overnight. The RM was concentrated in vacuum, then dissolved in 1 ml of DMSO and subjected to HPLC (2-10 min 25-50% water MeCN+FA 30/min; loading pump 4 ml/min MeCN column SunFire 19*100 mm). N-(4-amino-1H-pyrazolo[4,3-c]pyridin-7-yl)-2-oxo-2-[(2R,5S)-5-methyl-2-[2-(1-methyl-4-piperidyl)-7-quinolyl]-1-piperidyl]acetamide (10.3 mg, 17.99 μmol, 29.97% yield, HCOOH) was obtained.

Compound 65: ¹H NMR (600 MHz, dmso) δ 0.73-1.12 (m, 3H), 1.27-1.46 (m, 1H), 1.68-1.79 (m, 1H), 1.88-2.04 (m, 5H), 2.12-2.35 (m, 1H), 2.65-2.84 (m, 1H), 2.91-3.00 (m, 2H), 3.13-3.22 (m, 7H), 3.81-4.19 (m, 1H), 5.19-5.84 (m, 1H), 6.61-7.04 (m, 2H), 7.44-7.60 (m, 2H), 7.63-7.77 (m, 1H), 7.81-7.88 (m, 1H), 7.90-7.99 (m, 1H), 8.14-8.23 (m, 1H), 8.24-8.32 (m, 1H), 9.55-10.68 (m, 1H), 12.53-13.38 (m, 1H). LCMS(ESI): [M]⁺ m/z: calcd 526.2; found 527.2; Rt=1.777 min.

### Example 50. Synthesis of N-(8-amino-2,7-naphthyridin-4-yl)-2-((2R,5S)-5-methyl-2-(2-(1-methylpiperidin-4-yl)benzo[d]thiazol-5-yl)piperidin-1-yl)-2-oxoacetamide (Compound 41)

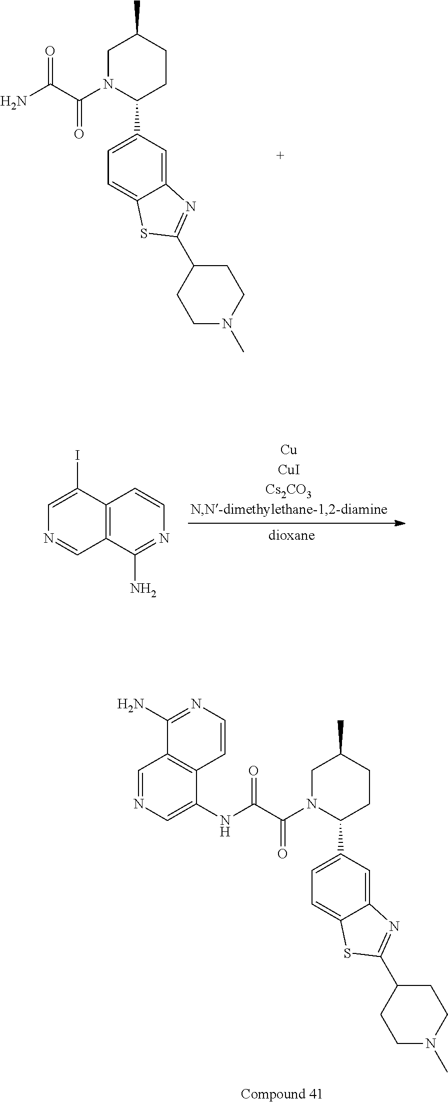 [SMILES: C[C@H]1CC[C@H](c2ccc3sc(C4CCN(C)CC4)nc3c2)N(C(=O)C(N)=O)C1 ; Nc1nccc2c(I)cncc12 ; C[C@H]1CC[C@H](c2ccc3sc(C4CCN(C)CC4)nc3c2)N(C(=O)C(=O)Nc2cncc3c(N)nccc23)C1]

The synthesis of 2-((2R,5S)-5-methyl-2-(2-(1-methylpiperidin-4-yl)benzo[d]thiazol-5-yl)piperidin-1-yl)-2-oxoacetamide is given in Intermediate 19.

The synthesis of 5-Iodo-2,7-naphthyridin-1-amine is given in Intermediate 41.

Prepared by general procedure Scheme S3 step 3A. Yield: 11 mg (6.36%).

HPLC conditions: Column: XBridge C18 100*19 mm, 5 microM; 0-5 min 10-35% water-MeCN+0.1% NH₄OH; (loading pump 4 ml/min MeCN).

Compound 41: ¹H NMR (500 MHz, DMSO-d₆) δ (ppm) 1.07-1.15 (m, 3H), 1.33-1.42 (m, 1H), 1.71-1.83 (m, 3H), 1.88-1.97 (m, 1H), 2.03-2.13 (m, 5H), 2.20 (s, 3H), 2.21-2.28 (m, 1H), 2.29-2.35 (m, 1H), 2.83-2.88 (m, 2H), 3.04-3.10 (m, 1H), 3.51-4.14 (m, 1H), 5.25-5.80 (m, 1H), 6.60-6.98 (m, 1H), 7.30-7.48 (m, 3H), 7.81-7.92 (m, 1H), 7.96-8.09 (m, 2H), 8.60-8.83 (m, 1H), 9.31-9.47 (m, 1H), 10.72-11.07 (m, 1H).

LCMS(ESI): [M]⁺ m/z: calcd 543.2; found 544.2; Rt=2.069 min.

### Example 51. Synthesis of 2-((2R,5S)-5-methyl-2-(2-(2-(pyrrolidin-1-yl)ethyl)benzo[d]thiazol-5-yl)piperidin-1-yl)-2-oxo-N-(1H-pyrazolo[4,3-c]pyridin-7-yl)acetamide (Compound 63)

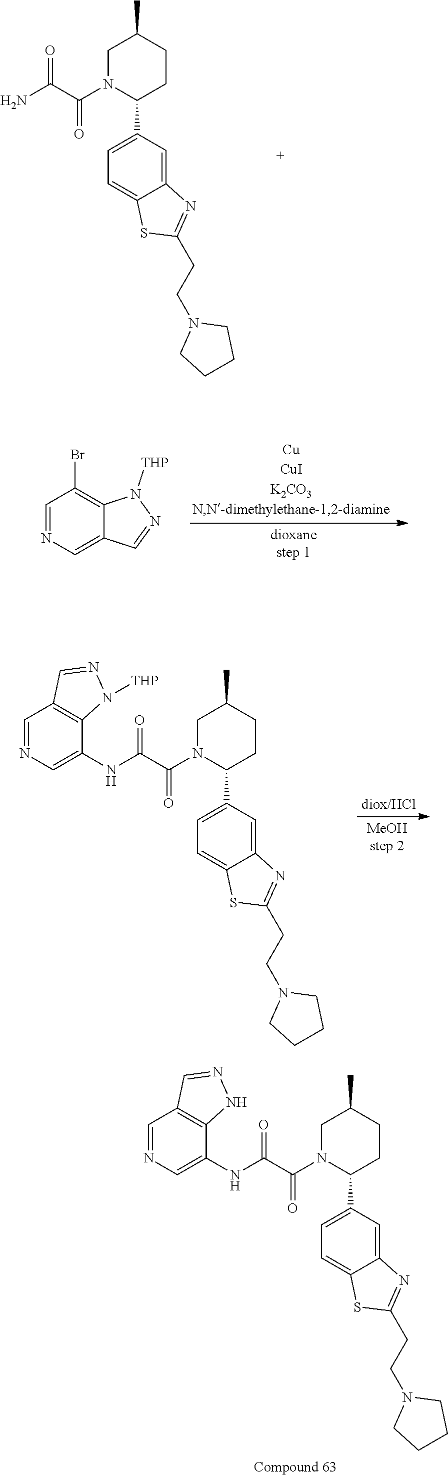

The synthesis of f 2-((2R,5S)-5-methyl-2-(2-(2-(pyrrolidin-1-yl)ethyl)benzo[d]thiazol-5-yl)piperidin-1-yl)-2-oxoacetamide is given in Intermediate 23.

### Step 1: Synthesis of 2-((2R,5S)-5-methyl-2-(2-(2-(pyrrolidin-1-yl)ethyl)benzo[d]thiazol-5-yl)piperidin-1-yl)-2-oxo-N-(1-(tetrahydro-2H-pyran-2-yl)-1H-pyrazolo[4,3-c]pyridin-7-yl)acetamide

Prepared by general procedure Scheme S3 step 3A. Yield: 56 mg (18.64%).

HPLC conditions: Column: XBridge C18 100*19 mm, 5 microM; 0-5 min 50-100% water-MeOH+0.1% NH₄OH, flow: 30 ml/min; (loading pump 4 ml/min MeOH).

LCMS(ESI): [M]⁺ m/z: calcd 601.2; found 602.2; Rt=1.877 min.

### Step 2: Synthesis of 2-((2R,5S)-5-methyl-2-(2-(2-(pyrrolidin-1-yl)ethyl)benzo[d]thiazol-5-yl)piperidin-1-yl)-2-oxo-N-(1H-pyrazolo[4,3-c]pyridin-7-yl)acetamide (Compound 63)

To a solution of 2-[(2R,5S)-5-methyl-2-[2-(2-pyrrolidin-1-ylethyl)-1,3-benzothiazol-5-yl]-1-piperidyl]-2-oxo-N-(1-tetrahydropyran-2-ylpyrazolo[4,3-c]pyridin-7-yl)acetamide (56 mg, 93.06 μmol) in MeOH (2 mL) was added hydrogen chloride solution 4.0M in dioxane (1.36 g, 37.22 mmol, 1.70 mL) at 21° C. The resulting mixture was left to stir for 15 min. The resulting mixture was evaporated to dryness and subjected to HPLC (5-5-20% 0-1-5 min H₂O/MeCN/0.1% FA, flow: 30 ml/min; column: Chromatorex 18 SMB100-5T 100×19 mm 5 um) to give Compound 63 2-[(2R,5S)-5-methyl-2-[2-(2-pyrrolidin-1-ylethyl)-1,3-benzothiazol-5-yl]-1-piperidyl]-2-oxo-N-(1H-pyrazolo[4,3-c]pyridin-7-yl)acetamide (38 mg, 60.60 μmol, 65.12% yield, ³HCl).

Compound 63: ¹H NMR (600 MHZ, DMSO-d₆) δ (ppm) δ 1.03-1.10 (m, 3H), 1.33-1.43 (m, 1H), 1.64-1.80 (m, 6H), 1.86-1.96 (m, 1H), 2.13-2.34 (m, 2H), 2.83-2.91 (m, 3H), 3.22-3.26 (m, 5H), 3.75-4.13 (m, 1H), 5.56-5.81 (m, 1H), 7.35-7.44 (m, 1H), 7.87-7.92 (m, 1H), 7.98-8.08 (m, 1H), 8.17 (s, 1H), 8.29-8.38 (m, 1H), 8.41-8.55 (m, 1H), 8.87-8.99 (m, 1H), 11.09-11.26 (m, 1H), 13.00-13.21 (m, 1H). LCMS(ESI): [M]⁺ m/z: calcd 517.2; found 518.2; Rt=1.785 min.

### Example 52. Alternative synthesis of 2-((2R,5S)-5-methyl-2-(2-(2-(pyrrolidin-1-yl)ethyl)benzo[d]thiazol-5-yl)piperidin-1-yl)-2-oxo-N-(1H-pyrazolo[4,3-c]pyridin-7-yl)acetamide (Compound 63)

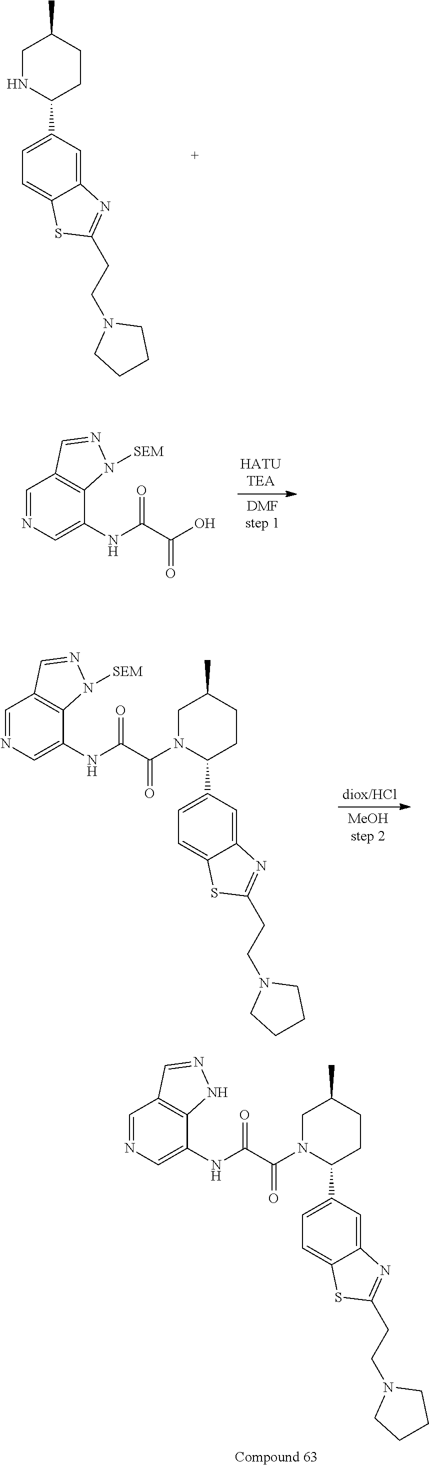 [SMILES: C[C@H]1CC[C@H](c2ccc3sc(CCN4CCCC4)nc3c2)NC1 ; C[Si](C)(C)CCOCn1ncc2cncc(NC(=O)C(=O)O)c21 ; C[C@H]1CC[C@H](c2ccc3sc(CCN4CCCC4)nc3c2)N(C(=O)C(=O)Nc2cncc3cnn(COCC[Si](C)(C)C)c23)C1 ; C[C@H]1CC[C@H](c2ccc3sc(CCN4CCCC4)nc3c2)N(C(=O)C(=O)Nc2cncc3cn[nH]c23)C1]

The synthesis of 5-((2R,5S)-5-methylpiperidin-2-yl)-2-(2-(pyrrolidin-1-yl)ethyl)benzo[d]thiazole is given in Intermediate 23.

### Step 1: Synthesis of 2-((2R,5S)-5-methyl-2-(2-(2-(pyrrolidin-1-yl)ethyl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure Scheme S3 step 3A. Yield: 223 mg (20.16%).

HPLC conditions: Column: XBridge C18 100*19 mm, 5 microM; 0-5 min 45-70% water-MeCN+0.1% NH₄OH, flow: 30 ml/min; (loading pump 4 ml/min MeCN).

LCMS(ESI): [M]⁺ m/z: calcd 647.2; found 648.2; Rt=3.364 min.

### Step 2: Synthesis of 2-((2R,5S)-5-methyl-2-(2-(2-(pyrrolidin-1-yl)ethyl)benzo[d]thiazol-5-yl)piperidin-1-yl)-2-oxo-N-(1H-pyrazolo[4,3-c]pyridin-7-yl)acetamide (Compound 63)

To a solution of 2-oxo-2-[(2R,5S)-5-methyl-2-[2-(2-pyrrolidin-1-ylethyl)-1,3-benzothiazol-5-yl]-1-piperidyl]-N-[1-(2-trimethylsilylethoxymethyl)pyrazolo[4,3-c]pyridin-7-yl]acetamide (223 mg, 344.19 μmol) in methanol (3 mL) was added hydrogen chloride solution 4.0M in dioxane (1.88 g, 5.16 mmol, 2.35 mL, 10% purity) at 21° C. The resulting mixture was left to stir for 18 hr. The resulting mixture was evaporated to dryness. The residue was purified by RP-HPLC (column: YMC Triart C18 100×20 mm, 5 um; 50-50-80% 0-1.5-6 min H₂O/MeOH/0.1% NH₄OH, flow: 30 ml/min) to give Compound 63 2-oxo-N-(1H-pyrazolo[4,3-c]pyridin-7-yl)-2-[(2R,5S)-5-methyl-2-[2-(2-pyrrolidin-1-ylethyl)-1,3-benzothiazol-5-yl]-1-piperidyl]acetamide (97 mg, 187.39 μmol, 54.44% yield).

Compound 63: ¹H NMR (600 MHz, dmso) δ 0.83-1.08 (m, 3H), 1.29-1.45 (m, 1H), 1.65-1.72 (m, 4H), 1.73-1.82 (m, 1H), 1.85-2.01 (m, 1H), 2.09-2.26 (m, 1H), 2.29-2.37 (m, 1H), 2.52-2.55 (m, 2H), 2.85-2.90 (m, 2H), 3.20-3.29 (m, 4H), 3.36-3.37 (m, 1H), 3.76-4.13 (m, 1H), 5.58-5.93 (m, 1H), 7.36-7.45 (m, 1H), 7.87-7.94 (m, 1H), 7.98-8.09 (m, 1H), 8.28-8.40 (m, 1H), 8.41-8.58 (m, 1H), 8.84-9.04 (m, 1H), 11.05-11.27 (m, 1H), 12.94-13.34 (m, 1H). LCMS(ESI): [M]⁺ m/z: calcd 517.2; found 518.2; Rt=1.878 min.

### Example 53. Synthesis of 2-((2R,5S)-5-methyl-2-(2-(2-(pyrrolidin-1-yl)ethyl)benzo[d]thiazol-5-yl)piperidin-1-yl)-2-oxo-N-(1H-pyrazolo[3,4-c]pyridin-4-yl)acetamide (Compound 102)

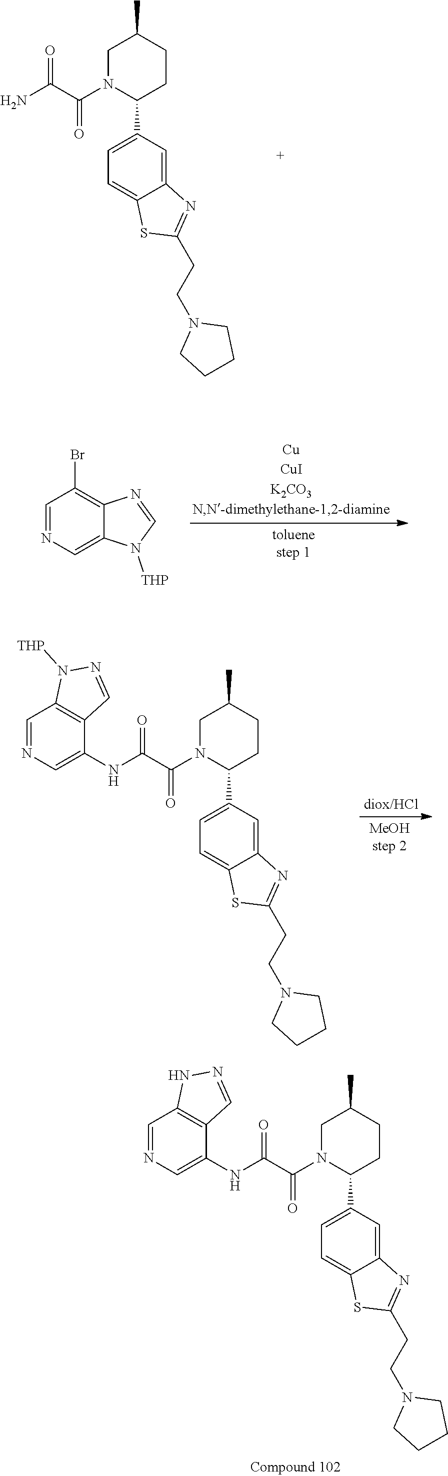

### Step 1: Synthesis of 2-((2R,5S)-5-methyl-2-(2-(2-(pyrrolidin-1-yl)ethyl)benzo[d]thiazol-5-yl)piperidin-1-yl)-2-oxo-N-(1-(tetrahydro-2H-pyran-2-yl)-1H-pyrazolo[3,4-c]pyridin-4-yl)acetamide

Prepared by general procedure Scheme S3 step 3A using Intermediate 23. Yield: 31 mg (17.19%).

HPLC conditions: Column: XBridge C18 100*19 mm, 5 microM; 0-5 min 50-50-80% water-MeOH+0.1% NH₄OH, flow: 30 ml/min; (loading pump 4 ml/min MeOH).

LCMS(ESI): [M]⁺ m/z: calcd 601.2; found 602.2; Rt=2.480 min.

### Step 2: Synthesis of 2-((2R,5S)-5-methyl-2-(2-(2-(pyrrolidin-1-yl)ethyl)benzo[d]thiazol-5-yl)piperidin-1-yl)-2-oxo-N-(1H-pyrazolo[3,4-c]pyridin-4-yl)acetamide (Compound 102)

To a solution of 2-[(2R,5S)-5-methyl-2-[2-(2-pyrrolidin-1-ylethyl)-1,3-benzothiazol-5-yl]-1-piperidyl]-2-oxo-N-(1-tetrahydropyran-2-ylpyrazolo[3,4-c]pyridin-4-yl)acetamide (31 mg, 51.52 μmol) in MeOH (1 mL) was added hydrogen chloride solution 4.0M in dioxane (751.32 mg, 20.61 mmol, 939.15 μL) at 21° C. The resulting mixture was left to stir for 15 min. The resulting mixture was evaporated to dryness and subjected to HPLC (5-5-20% 0-2-5 min H₂O/MeCN/0.1, % FA flow: 30 ml/min; column: Chromatorex 18 SMB100-5T 100×19 mm 5 um) to give 2-[(2R,5S)-5-methyl-2-[2-(2-pyrrolidin-1-ylethyl)-1,3-benzothiazol-5-yl]-1-piperidyl]-2-oxo-N-(1H-pyrazolo[3,4-c]pyridin-4-yl)acetamide (10.5 mg, 20.28 μmol, 39.37% yield).

Compound 102: ¹H NMR (600 MHz, DMSO-d₆) δ (ppm) 1.04-1.10 (m, 3H), 1.34-1.43 (m, 1H), 1.66-1.76 (m, 5H), 1.83-1.96 (m, 1H), 2.13-2.25 (m, 1H), 2.30-2.35 (m, 1H), 2.84-2.89 (m, 2H), 3.21-3.23 (m, 2H), 3.24-3.27 (m, 1H), 3.33-3.34 (m, 2H), 3.36-3.37 (m, 2H), 3.44-4.07 (m, 1H), 5.25-5.77 (m, 1H), 7.34-7.48 (m, 1H), 7.85-7.96 (m, 1H), 7.99-8.08 (m, 1H), 8.10-8.39 (m, 2H), 8.53-8.68 (m, 1H), 8.76-8.89 (m, 1H), 11.20-11.58 (m, 1H), 13.69 (s, 1H). LCMS(ESI): [M]⁺ m/z: calcd 517.2; found 518.2; Rt=2.261 min.

### Example 54. Synthesis of N-(4-amino-1H-pyrazolo[4,3-c]pyridin-7-yl)-2-((2R,5S)-2-(2-(1-(dimethylamino) propan-2-yl)benzo[d]thiazol-5-yl)-5-methylpiperidin-1-yl)-2-oxoacetamide (Compound 35)

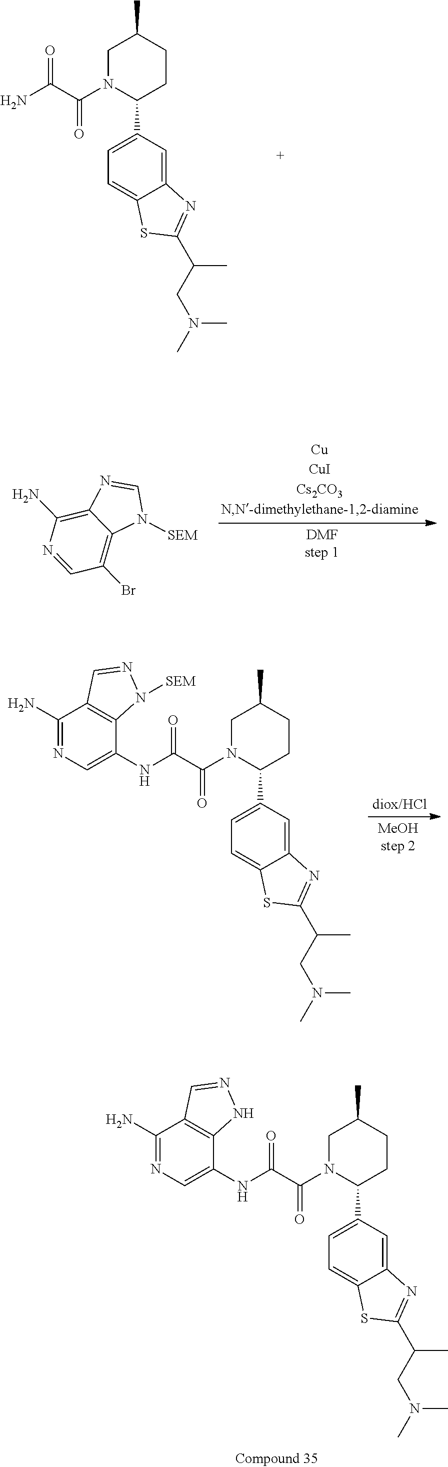 [SMILES: CC(CN(C)C)c1nc2cc([C@H]3CC[C@H](C)CN3C(=O)C(N)=O)ccc2s1 ; C[Si](C)(C)CCOCn1cnc2c(N)ncc(Br)c21 ; CC(CN(C)C)c1nc2cc([C@H]3CC[C@H](C)CN3C(=O)C(=O)Nc3cnc(N)c4cnn(COCC[Si](C)(C)C)c34)ccc2s1 ; CC(CN(C)C)c1nc2cc([C@H]3CC[C@H](C)CN3C(=O)C(=O)Nc3cnc(N)c4cn[nH]c34)ccc2s1]

The synthesis of 2-((2R,5S)-2-(2-(1-(dimethylamino) propan-2-yl)benzo[d]thiazol-5-yl)-5-methylpiperidin-1-yl)-2-oxoacetamide is given in Example 44

### Step 1: Synthesis of N-(4-amino-1-((2-(trimethylsilyl)ethoxy)methyl)-1H-pyrazolo[4,3-c]pyridin-7-yl)-2-((2R,5S)-2-(2-(1-(dimethylamino) propan-2-yl)benzo[d]thiazol-5-yl)-5-methylpiperidin-1-yl)-2-oxoacetamide

2-Oxo-2-[(2R,5S)-2-[2-[2-(dimethylamino)-1-methyl-ethyl]-1,3-benzothiazol-5-yl]-5-methyl-1-piperidyl]acetamide (0.2 g, 514.77 μmol), 7-bromo-1-(2-trimethylsilylethoxymethyl)pyrazolo[4,3-c]pyridin-4-amine (176.72 mg, 514.77 μmol), Cu (32.71 mg, 514.77 μmol), cesium carbonate (335.44 mg, 1.03 mmol), CuI (98.04 mg, 514.77 μmol, 17.44 μL) and N,N′-dimethylethane-1,2-diamine (109.83 mg, 772.15 μmol, 121.77 μL) were mixed in DMF (4 mL) under argon. The resulting mixture was allowed to stir at 100° C. for 17 hr in vial. Upon completion of the reaction, dioxane was evaporated and residue was subjected by HPLC (column: XBridge C18 100×19 mm, 5 um; mobile phase: 30-80% 0-5 min H₂O/MeOH/0.1% NH₄OH, flow rate: 30 ml/min (loading pump 4 ml/min MeCN), affording N-[4-amino-1-(2-trimethylsilylethoxymethyl)pyrazolo[4,3-c]pyridin-7-yl]-2-oxo-2-[(2R,5S)-2-[2-[2-(dimethylamino)-1-methyl-ethyl]-1,3-benzothiazol-5-yl]-5-methyl-1-piperidyl]acetamide (40.4 mg, 62.07 μmol, 12.06% yield) on two portions. LCMS(ESI): [M]⁺ m/z: calcd 650.2; found 651.2; Rt=2.890 min.

### Step 2: Synthesis of N-(4-amino-1H-pyrazolo[4,3-c]pyridin-7-yl)-2-((2R,5S)-2-(2-(1-(dimethylamino) propan-2-yl)benzo[d]thiazol-5-yl)-5-methylpiperidin-1-yl)-2-oxoacetamide (Compound 35)

N-[4-Amino-1-(2-trimethylsilylethoxymethyl)pyrazolo[4,3-c]pyridin-7-yl]-2-oxo-2-[(2R,5S)-2-[2-[2-(dimethylamino)-1-methyl-ethyl]-1,3-benzothiazol-5-yl]-5-methyl-1-piperidyl]acetamide (40.46 mg, 62.16 μmol) was dissolved in a mixture of diox/HCl (1 mL) and MeOH (1 mL), the resulting mixture was stirred for 17 hr at rt. Upon completion of the reaction, the resulting mixture was evaporated to dryness, to obtain crude product. Crude product was subjected by HPLC (column: Chromatorex 18 SMB100-5T 100×19 mm, 5 um; mobile phase: 0-25% 0-5 min H₂O/MeCN/0.1% FA, flow rate: 30 ml/min (loading pump 4 ml/min MeCN), affording N-(4-amino-1H-pyrazolo[4,3-c]pyridin-7-yl)-2-oxo-2-[(2R,5S)-2-[2-[2-(dimethylamino)-1-methyl-ethyl]-1,3-benzothiazol-5-yl]-5-methyl-1-piperidyl]acetamide (15.3 mg, 25.78 μmol, 41.47% yield, 2HCl).

Compound 35: ¹H NMR (DMSO-d6, 600 MHz): δ (ppm) 1.05 (m, 3H), 1.40 (m, 4H), 1.73 (m, 1H), 1.91 (m, 2H), 2.12 (m, 1H), 2.28 (m, 2H), 2.53 (m, 6H), 2.87 (m, 2H), 3.79 (m, 4H), 5.65 (m, 1H), 7.43 (m, 3H), 7.78 (m, 1H), 7.92 (m, 1H), 8.12 (m, 2H), 8.26 (m, 1H), 10.70 (m, 1H), 13.06 (m, 1H). LCMS(ESI): [M]⁺ m/z: calcd 519.2; found 520.2; Rt=2.106 min.

### Example 55. Synthesis of 2-((2R,5S)-5-methyl-2-(2-(2-methyl-2-azaspiro[3.3]heptan-6-yl)benzo[d]thiazol-5-yl)piperidin-1-yl)-2-oxo-N-(1H-pyrazolo[4,3-c]pyridin-7-yl)acetamide (Compound 93)

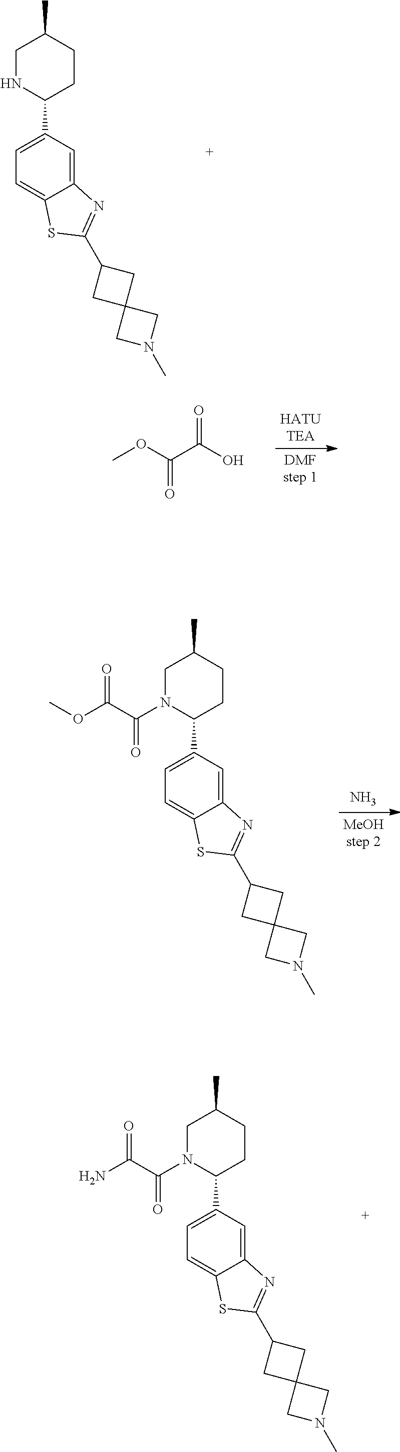

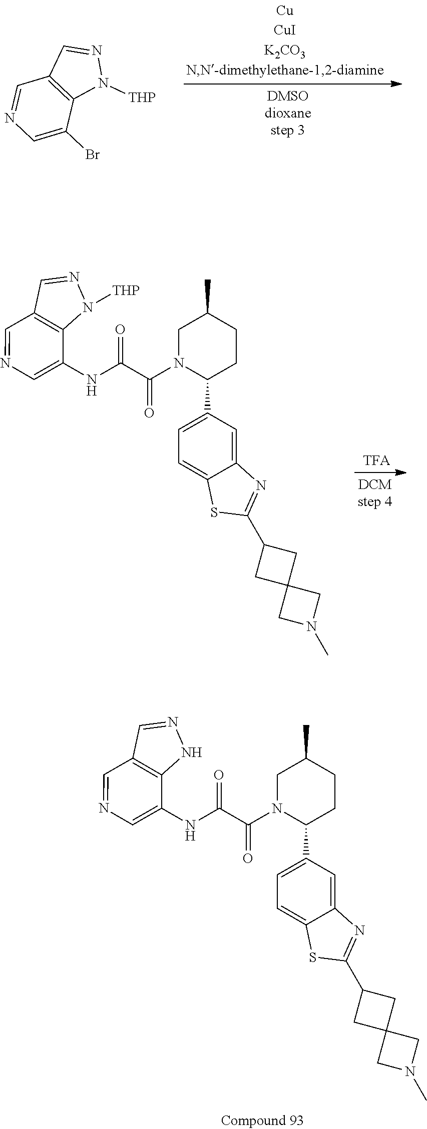

The synthesis of 2-(2-methyl-2-azaspiro[3.3]heptan-6-yl)-5-((2R,5S)-5-methylpiperidin-2-yl)benzo[d]thiazole is given in Intermediate 13.

### Step 1: Synthesis of methyl 2-((2R,5S)-5-methyl-2-(2-(2-methyl-2-azaspiro[3.3]heptan-6-5 yl)benzo[d]thiazol-5-yl)piperidin-1-yl)-2-oxoacetate

To a stirred mixture of 2-(2-methyl-2-azaspiro[3.3]heptan-6-yl)-5-[(2R,5S)-5-methyl-2-piperidyl]-1,3-benzothiazole (975 mg, 2.85 mmol), 2-methoxy-2-oxo-acetic acid (445.63 mg, 4.28 mmol) and TEA (1.44 g, 14.27 mmol, 1.99 mL) in DMF (10 mL) was added HATU (1.74 g, 4.57 mmol, 2.39 mL). The resulting reaction mixture was stirred at 20° C. for 4 hr. Then, it was diluted with water (40 ml) and extracted with ethyl acetate (70 ml). Organic layer was washed successively with water (2×30 ml) and brine (30 ml), dried over Na₂SO₄ and concentrated under reduced pressure, affording methyl 2-oxo-2-[(2R,5S)-5-methyl-2-[2-(2-methyl-2-azaspiro[3.3]heptan-6-yl)-1,3-benzothiazol-5-yl]-1-piperidyl]acetate (1.24 g, crude).

LCMS(ESI): [M]⁺ m/z: calcd 427.2; found 428.2; Rt=1.051 min.

### Step 2: Synthesis of 2-((2R,5S)-5-methyl-2-(2-(2-methyl-2-azaspiro[3.3]heptan-6-yl)benzo[d]thiazol-5-yl)piperidin-1-yl)-2-oxoacetamide

Methyl 2-oxo-2-[(2R,5S)-5-methyl-2-[2-(2-methyl-2-azaspiro[3.3]heptan-6-yl)-1,3-benzothiazol-5-yl]-1-piperidyl]acetate (1.24 g, 2.90 mmol) was dissolved in ammonia (7N in MeOH)(15.58 g, 139.97 mmol, 20 mL, 15.3% purity). Resulting solution was stirred at 45° C. for 16 hr. Then, solvent was removed under reduced pressure, leaving 2-oxo-2-[(2R,5S)-5-methyl-2-[2-(2-methyl-2-azaspiro[3.3]heptan-6-yl)-1,3-benzothiazol-5-yl]-1-piperidyl]acetamide (1.21 g, crude). LCMS(ESI): [M]⁺ m/z: calcd 412.2; found 413.2; Rt=0.862 min.

### Step 3: Synthesis of 2-((2R,5S)-5-methyl-2-(2-(2-methyl-2-azaspiro[3.3]heptan-6-yl)benzo[d]thiazol-5-yl)piperidin-1-yl)-2-oxo-N-(1-(tetrahydro-2H-pyran-2-yl)-1H-pyrazolo[4,3-c]pyridin-7-yl)acetamide

2-Oxo-2-[(2R,5S)-5-methyl-2-[2-(2-methyl-2-azaspiro[3.3]heptan-6-yl)-1,3-benzothiazol-5-yl]-1-piperidyl]acetamide (600 mg, 1.45 mmol), 7-bromo-1-tetrahydropyran-2-yl-pyrazolo[4,3-c]pyridine (492.40 mg, 1.75 mmol), copper (18.49 mg, 290.88 μmol), copper (I) iodide (83.10 mg, 436.31 μmol, 14.79 μL), N,N′-dimethylethanediamine (62.06 mg, 436.31 μmol, 68.80 μL) and potassium carbonate (402.02 mg, 2.91 mmol, 175.55 μL) were mixed together in dioxane (15 mL) and DMSO (5 mL). Reaction flask was purged with argon and resulting mixture was stirred at 105° C. for 20 hr under inert atmosphere. Then, dioxane was removed under reduced pressure and residue was subjected to HPLC (40-40-90% 0-1-6 min H₂O/MeOH/0.1% NH₄OH, flow: 30 ml/min; column: YMC Triart C18 100×20 mm, 5 um), affording 2-oxo-2-[(2R,5S)-5-methyl-2-[2-(2-methyl-2-azaspiro[3.3]heptan-6-yl)-1,3-benzothiazol-5-yl]-1-piperidyl]-N-(1-tetrahydropyran-2-ylpyrazolo[4,3-c]pyridin-7-yl)acetamide (240 mg, 391.03 μmol, 26.89% yield). LCMS(ESI): [M]⁺ m/z: calcd 613.2; found 614.2; Rt=0.992 min.

### Step 4: Synthesis of 2-((2R,5S)-5-methyl-2-(2-(2-methyl-2-azaspiro[3.3]heptan-6-yl)benzo[d]thiazol-5-yl)piperidin-1-yl)-2-oxo-N-(1H-pyrazolo[4,3-c]pyridin-7-yl)acetamide (Compound 93)

TFA (1.48 g, 12.98 mmol, 1 mL) was added to the solution of 2-oxo-2-[(2R,5S)-5-methyl-2-[2-(2-methyl-2-azaspiro[3.3]heptan-6-yl)-1,3-benzothiazol-5-yl]-1-piperidyl]-N-(1-tetrahydropyran-2-ylpyrazolo[4,3-c]pyridin-7-yl)acetamide (240 mg, 391.03 μmol) in DCM (5 mL). Resulting mixture was stirred at 25° C. for 6 hr. Then, volatiles were removed under reduced pressure and residue was subjected to HPLC (1-st run: 5-5-40% 0-2-5 min H₂O/MeCN/0.2% FA, flow: 30 ml/min; column: Chromatorex 18 SMB 100-5T 100×19 mm 5 um; 2-nd run: 40-40-80% 0-1-6 min H₂O/MeOH/0.1% NH₄OH, flow: 30 ml/min; column: YMC Triart C18 100×20 mm, 5 um), affording 2-oxo-N-(1H-pyrazolo[4,3-c]pyridin-7-yl)-2-[(2R,5S)-5-methyl-2-[2-(2-methyl-2-azaspiro[3.3]heptan-6-yl)-1,3-benzothiazol-5-yl]-1-piperidyl]acetamide (58 mg, 109.51 μmol, 28.00% yield).

Compound 93: ¹H NMR (600 MHZ, DMSO-d₆) δ (ppm) 1.01-1.10 (m, 3H), 1.31-1.44 (m, 1H), 1.69-1.80 (m, 1H), 1.83-1.97 (m, 1H), 2.11-2.34 (m, 5H), 2.43-2.46 (m, 2H), 2.57-2.92 (m, 3H), 3.03-3.08 (m, 2H), 3.20-3.23 (m, 2H), 3.76-4.13 (m, 2H), 5.52-5.78 (m, 1H), 7.35-7.45 (m, 1H), 7.88-7.94 (m, 1H), 7.98-8.08 (m, 1H), 8.29-8.38 (m, 1H), 8.41-8.55 (m, 1H), 8.87-8.99 (m, 1H), 10.88-11.37 (m, 1H), 12.83-13.29 (m, 1H).

LCMS(ESI): [M]⁺ m/z: calcd 529.2; found 530.2; Rt=2.108 min.

### Example 56. Synthesis of 2-((2R,5S)-5-methyl-2-(2-(1,2,2,6,6-pentamethylpiperidin-4-yl)benzo[d]thiazol-5-yl)piperidin-1-yl)-2-oxo-N-(1H-pyrazolo[4,3-c]pyridin-7-yl)acetamide (Compound 85)

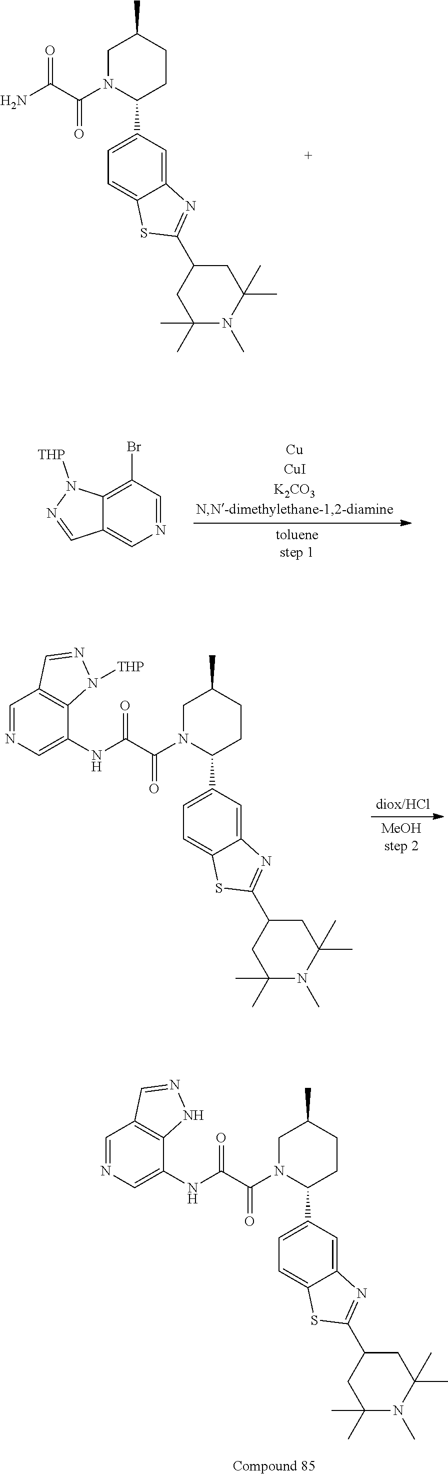

The synthesis of 5-((2R,5S)-5-methylpiperidin-2-yl)-2-(1,2,2,6,6-pentamethylpiperidin-4-yl)benzo[d]thiazole is given in Intermediate 7. Oxamide was formed according to procedures known in the art (see for example Intermediate 26 and Intermediate 28).

### Step 1: Synthesis of 2-((2R,5S)-5-methyl-2-(2-(1,2,2,6,6-pentamethylpiperidin-4-yl)benzo[d]thiazol-5-yl)piperidin-1-yl)-2-oxo-N-(1-(tetrahydro-2H-pyran-2-yl)-1H-pyrazolo[4,3-c]pyridin-7-yl)acetamide

Prepared by general procedure Scheme S3 step 3A. Yield: 2120 mg (49%).

HPLC conditions: Column: Chromatorex C18 100*19 mm, 5 microM; 0-1-6 min 60-60-100% water-MeOH+0.1% NH₄OH; (loading pump 4 ml/min MeOH).

LCMS(ESI): [M]⁺ m/z: calcd 657.2; found 658.2; Rt=1.700 min.

### Step 2: Synthesis of 2-((2R,5S)-5-methyl-2-(2-(1,2,2,6,6-pentamethylpiperidin-4-yl)benzo[d]thiazol-5-yl)piperidin-1-yl)-2-oxo-N-(1H-pyrazolo[4,3-c]pyridin-7-yl)acetamide (Compound 85)

To a solution of 2-oxo-2-[(2R,5S)-5-methyl-2-[2-(1,2,2,6,6-pentamethyl-4-piperidyl)-1,3-benzothiazol-5-yl]-1-piperidyl]-N-(1-tetrahydropyran-2-ylpyrazolo[4,3-c]pyridin-7-yl)acetamide (0.12 g, 182.41 μmol) in MeOH (9.99 mL) was added hydrogen chloride solution 4.0M in dioxane (1.60 g, 43.88 mmol, 2 mL) at 21° C. The resulting mixture was left to stir for 8 hr. The resulting mixture was evaporated to dryness and subjected to HPLC (25-60% 0-6 min H₂O/MeCN/0.1, % NH₄OH flow: 30 ml/min; column: XBridge C 18 100×20 mm 5 um) to afford 2-oxo-N-(1H-pyrazolo[4,3-c]pyridin-7-yl)-2-[(2R,5S)-5-methyl-2-[2-(1,2,2,6,6-pentamethyl-4-piperidyl)-1,3-benzothiazol-5-yl]-1-piperidyl]acetamide (0.0161 g, 28.06 μmol, 15.38% yield).

Compound 85: ¹H NMR (500 MHz, dmso) δ 1.04-1.08 (m, 9H), 1.11-1.15 (m, 6H), 1.16-1.32 (m, 1H), 1.32-1.44 (m, 1H), 1.58-1.66 (m, 2H), 1.68-1.78 (m, 1H), 1.81-1.91 (m, 1H), 1.91-2.04 (m, 3H), 2.09-2.18 (m, 1H), 2.21 (s, 3H), 2.79-2.90 (m, 1H), 3.49-3.53 (m, 2H), 3.76-4.13 (m, 1H), 5.55-5.82 (m, 1H), 7.37-7.50 (m, 1H), 7.87-7.97 (m, 1H), 7.99-8.10 (m, 1H), 8.23-8.34 (m, 1H), 8.35-8.51 (m, 1H), 8.78-8.94 (m, 1H).

LCMS(ESI): [M]⁺ m/z: calcd 573.2; found 574.2; Rt=1.466 min.

### Example 57. Synthesis of N-(4-amino-1H-pyrazolo[4,3-c]pyridin-7-yl)-2-((2R,5S)-5-methyl-2-(2-(1-methylazetidin-3-yl)benzo[d]thiazol-5-yl)piperidin-1-yl)-2-oxoacetamide (Compound 107)

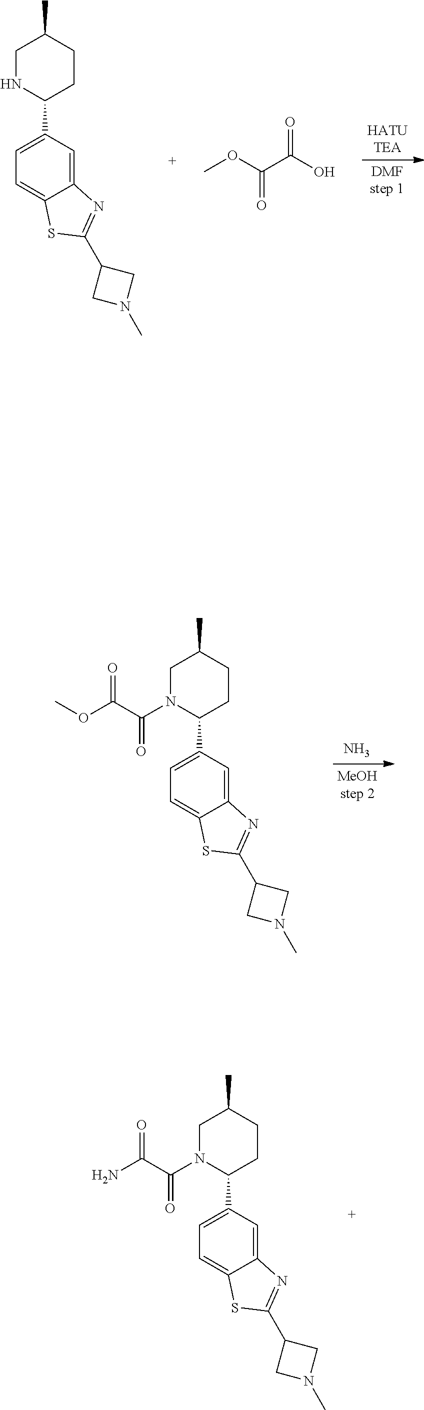

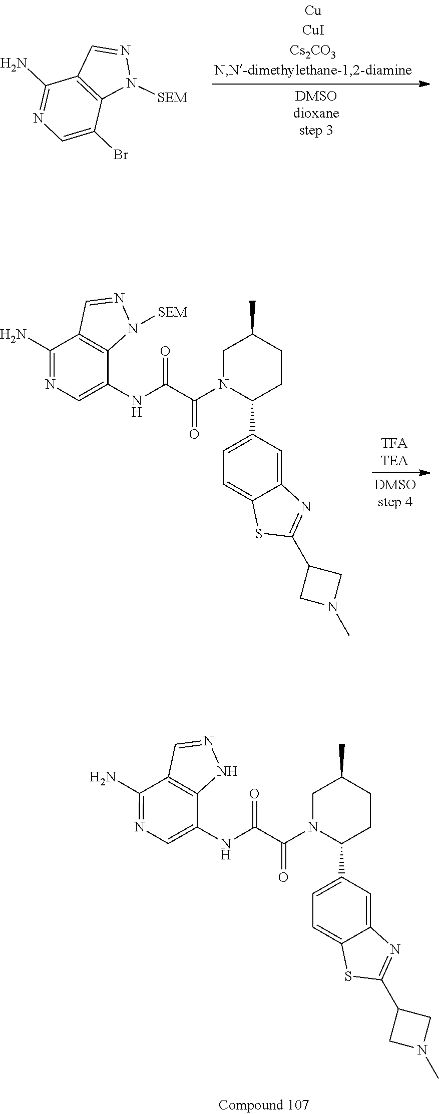 [SMILES: C[Si](C)(C)CCOCn1ncc2c(N)ncc(Br)c21 ; C[C@H]1CC[C@H](c2ccc3sc(C4CN(C)C4)nc3c2)N(C(=O)C(=O)Nc2cnc(N)c3cnn(COCC[Si](C)(C)C)c23)C1 ; C[C@H]1CC[C@H](c2ccc3sc(C4CN(C)C4)nc3c2)N(C(=O)C(=O)Nc2cnc(N)c3cn[nH]c23)C1]

The synthesis of 2-(1-methylazetidin-3-yl)-5-((2R,5S)-5-methylpiperidin-2-yl)benzo[d]thiazole is given in Intermediate 6.

### Step 1: Synthesis of 2-((2R,5S)-5-methyl-2-(2-(1-methylazetidin-3-yl)benzo[d]thiazol-5-5 yl)piperidin-1-yl)-2-oxoacetic acid

HATU (832.49 mg, 2.19 mmol) was added in small portions at 25° C. to a stirred mixture of 2-(1-methylazetidin-3-yl)-5-[(2R,5S)-5-methyl-2-piperidyl]-1,3-benzothiazole (0.55 g, 1.82 mmol), 2-methoxy-2-oxo-acetic acid (284.79 mg, 2.74 mmol) and DIPEA (707.42 mg, 5.47 mmol, 953.39 μL) in DMF (5 mL). The resulting mixture was stirred at 25° C. for 2 hr. Volatiles was evaporated, residue mixed with water (20 mL), extracted with EtOAc (2×10 mL), combined organic layer washed with water (2×5 mL), dried over Na₂SO₄ to give crude 2-((2R,5S)-5-methyl-2-(2-(1-methylazetidin-3-yl)benzo[d]thiazol-5-yl)piperidin-1-yl)-2-oxoacetic acid (0.5 g, 1.34 mmol, 73.38% yield). LCMS(ESI): [M]⁺ m/z: calcd 387.2; found 388.2; Rt=2.468 min.

### Step 2: Synthesis of 2-((2R,5S)-5-methyl-2-(2-(1-methylazetidin-3-yl)benzo[d]thiazol-5-yl)piperidin-1-yl)-2-oxoacetamide

Ammonia (114.00 mg, 6.69 mmol) was bubbled through a solution of methyl 2-oxo-2-[(2R,5S)-5-methyl-2-[2-(1-methylazetidin-3-yl)-1,3-benzothiazol-5-yl]-1-piperidyl]acetate (518.78 mg, 1.34 mmol) in MeOH (10 mL) for 0.5 hr at 0° C., then reaction mixture was stirred at 30° C. for 14 hr in closed flask. Volatiles was evaporated and residue submitted to HPLC (column: YMC Triart C18 100×20 mm, 5 um; mobile phase 15-30% 0-6 min H₂O/MeCN/0.1% NH₄OH, flow rate: 30 ml/min) to give pure 2-oxo-2-[(2R,5S)-5-methyl-2-[2-(1-methylazetidin-3-yl)-1,3-benzothiazol-5-yl]-1-piperidyl]acetamide (0.05 g, 134.23 μmol, 10.03% yield). LCMS(ESI): [M]⁺ m/z: calcd 372.2; found 373.2; Rt=2.100 min.

### Step 3: Synthesis of N-(4-amino-1-((2-(trimethylsilyl)ethoxy)methyl)-1H-pyrazolo[4,3-c]pyridin-7-yl)-2-((2R,5S)-5-methyl-2-(2-(1-methylazetidin-3-yl)benzo[d]thiazol-5-yl)piperidin-1-yl)-2-oxoacetamide

A mixture of 2-oxo-2-[(2R,5S)-5-methyl-2-[2-(1-methylazetidin-3-yl)-1,3-benzothiazol-5-yl]-1-piperidyl]acetamide (50 mg, 114.10 μmol), 7-bromo-1-(2-trimethylsilylethoxymethyl)pyrazolo[4,3-c]pyridin-4-amine (43.09 mg, 125.51 μmol), copper (5.26 mg, 82.82 μmol), copper (I) iodide (10.53 mg, 55.27 μmol, 1.87 μL), cesium carbonate (111.53 mg, 342.30 μmol) and N¹,N²-dimethylethane-1,2-diamine (31.65 mg, 222.49 μmol) in dioxane (4.00 mL) was stirred at 90° C. for 80 hr under argon atmosphere. After 12 hr aliquot was taken and showed 30% conversion. DMSO (1 mL) were added to a reaction mixture and stirred at 100° C. for 80 hr. Reaction mixture cooled down and filtered. The filter cake washed with dioxane (2×1 ml) and discarded. The combined filtrate was concentrated to give crude N-[4-amino-1-(2-trimethylsilylethoxymethyl)pyrazolo[4,3-c]pyridin-7-yl]-2-oxo-2-[(2R,5S)-5-methyl-2-[2-(1-methylazetidin-3-yl)-1,3-benzothiazol-5-yl]-1-piperidyl]acetamide (72 mg, 113.41 μmol, 99.40% yield) (solution in DMSO (1 mL). LCMS(ESI): [M]⁺ m/z: calcd 634.2; found 635.2; Rt=2.810 min.

### Step 4: Synthesis of N-(4-amino-1H-pyrazolo[4,3-c]pyridin-7-yl)-2-((2R,5S)-5-methyl-2-(2-(1-methylazetidin-3-yl)benzo[d]thiazol-5-yl)piperidin-1-yl)-2-oxoacetamide (Compound 107)

TFA (1.26 g, 11.03 mmol, 849.45 μL) was added to a solution of N-[4-amino-1-(2-trimethylsilylethoxymethyl)pyrazolo[4,3-c]pyridin-7-yl]-2-oxo-2-[(2R,5S)-5-methyl-2-[2-(1-methylazetidin-3-yl)-1,3-benzothiazol-5-yl]-1-piperidyl]acetamide (70 mg, 110.26 μmol) in DMSO (923.09 μL) and stirred at 25° C. for 24 hr. Volatiles was evaporated, residue cooled to 0° C., TEA (55.79 mg, 551.30 μmol, 76.84 μL) was added, concentrated in vacuum and submitted to HPLC (column: XBridge C18 100×19 mm, 5 um; mobile phase: 25-25-75% 0-1-8 min H₂O/MeOH/0.1% NH₄OH, flow rate: 30 ml/min) to give crude product (25 mg, 76%). After 2-nd HPLC (column: Chromatorex 18 SMB100-5T 100×19 mm 5 um; mobile phase: 0-0-25% 0-1-5 min H₂O/MeCN/0.1% FA, flow rate: 30 ml/min) pure N-(4-amino-1H-pyrazolo[4,3-c]pyridin-7-yl)-2-oxo-2-[(2R,5S)-5-methyl-2-[2-(1-methylazetidin-3-yl)-1,3-benzothiazol-5-yl]-1-piperidyl]acetamide (15 mg, 27.24 μmol, 24.71% yield, HCOOH) was obtained.

Compound 107: ¹H NMR (600 MHz, dmso) δ 0.93-1.07 (m, 3H), 1.32-1.43 (m, 1H), 1.67-1.79 (m, 1H), 1.85-1.95 (m, 1H), 2.09-2.23 (m, 1H), 2.30 (s, 3H), 2.76-3.07 (m, 3H), 3.68-3.72 (m, 2H), 3.74-4.13 (m, 3H), 5.12-5.80 (m, 1H), 6.62-7.01 (m, 2H), 7.29-7.48 (m, 1H), 7.62-7.79 (m, 1H), 7.89-7.96 (m, 1H), 7.99-8.10 (m, 1H), 8.14-8.21 (m, 1H), 10.48-10.63 (m, 1H), 12.55-13.40 (m, 1H). LCMS(ESI): [M]⁺ m/z: calcd 504.2; found 505.2; Rt=1.952 min.

### Example 58. Synthesis of 2-(2-(2-(2-(dimethylamino)ethyl)benzo[d]thiazol-5-yl)-5-methylpiperidin-1-yl)-N-(imidazo[1,2-a]pyridin-7-yl)-2-oxoacetamide (Compound 16 and Compound 27)

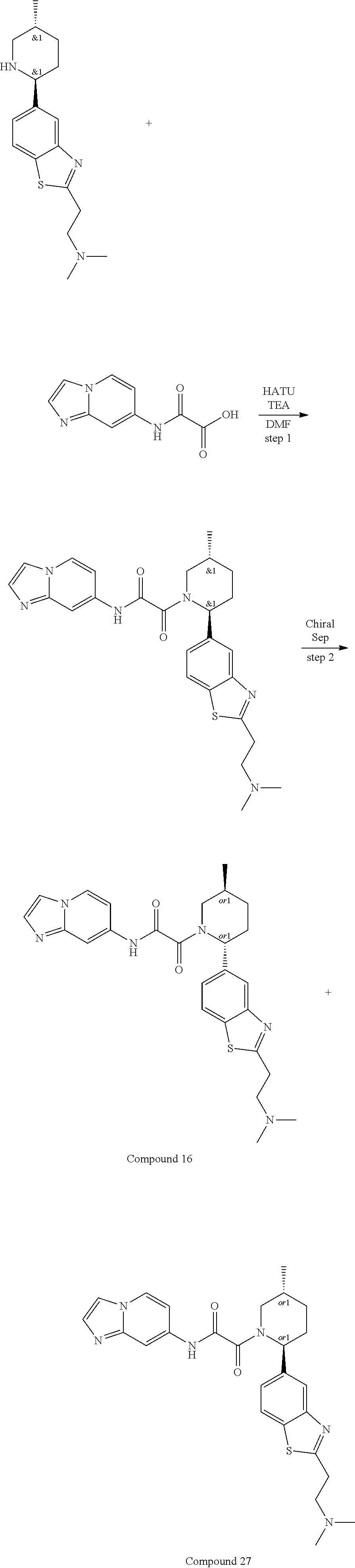 [SMILES: C[C@@H]1CC[C@@H](c2ccc3sc(CCN(C)C)nc3c2)NC1 ; O=C(O)C(=O)Nc1ccn2ccnc2c1 ; C[C@@H]1CC[C@@H](c2ccc3sc(CCN(C)C)nc3c2)N(C(=O)C(=O)Nc2ccn3ccnc3c2)C1 ; C[C@H]1CC[C@H](c2ccc3sc(CCN(C)C)nc3c2)N(C(=O)C(=O)Nc2ccn3ccnc3c2)C1 ; C[C@@H]1CC[C@@H](c2ccc3sc(CCN(C)C)nc3c2)N(C(=O)C(=O)Nc2ccn3ccnc3c2)C1]

The synthesis of rac-N,N-dimethyl-2-(5-((2R,5S)-5-methylpiperidin-2-yl)benzo[d]thiazol-2-yl)ethanamine is given in Intermediate 24.

### Step 1: Synthesis of 2-(2-(2-(2-(dimethylamino)ethyl)benzo[d]thiazol-5-yl)-5-methylpiperidin-1-yl)-N-(imidazo[1,2-a]pyridin-7-yl)-2-oxoacetamide

Prepared by general procedure Scheme S4 step 6A. Yield: 243 mg (40.65%).

HPLC conditions: Column: Chromatorex C18 100*19 mm, 5 microM; 0-5 min 35-75% water-MeOH, flow: 30 ml/min; (loading pump 4 ml/min MeOH).

LCMS(ESI): [M]⁺ m/z: calcd 490.2; found 491.2; Rt=2.093 min.

### Step 2: Chiral Separation (Compound 16 and Compound 27)

Racemic 2-(2-(2-(2-(dimethylamino)ethyl)benzo[d]thiazol-5-yl)-5-methylpiperidin-1-yl)-N-(imidazo[1,2-a]pyridin-7-yl)-2-oxoacetamide (57 mg, 118.60 μmol) was chiral separated (Column: Chiralpak IA-II (250*20, 5 mkm), MeOH-IPA, 50-50, 10 ml/min) to obtain N-(6-amino-5-methyl-3-pyridyl)-2-[rel-(2S,5R)-2-[2-[2-(dimethylamino)ethyl]-1,3-benzothiazol-5-yl]-5-methyl-1-piperidyl]-2-oxo-acetamide (18.7 mg, 38.91 μmol, 65.61% yield) (RT=33.47 min) and N-(6-amino-5-methyl-3-pyridyl)-2-[rel-(2R,5S)-2-[2-[2-(dimethylamino)ethyl]-1,3-benzothiazol-5-yl]-5-methyl-1-piperidyl]-2-oxo-acetamide (20 mg, 41.61 μmol, 70.18% yield) (RT=57.96 min).

Rel Time for Compound 16 in analytical conditions (column: OJ-H, Hexane-IPA-MeOH, 60-20-20, 0.6 ml/min as mobile phase) 15.71 min and for Compound 27 37.52 min.

Compound 16: Retention time: 15.71 min. ¹H NMR (600 MHZ, DMSO-d₆) δ (ppm) 1.03-1.06 (m, 3H), 1.33-1.40 (m, 1H), 1.69-1.73 (m, 1H), 1.87-1.92 (m, 1H), 2.09-2.32 (m, 8H), 2.78-2.89 (m, 2H), 3.23-3.25 (m, 2H), 3.48-4.05 (m, 2H), 5.29-5.72 (m, 1H), 7.01-7.10 (dd, 1H), 7.35-7.42 (dd, 1H), 7.45-7.50 (d, 1H), 7.81-7.89 (m, 2H), 8.02-8.12 (m, 2H), 8.41-8.50 (dd, 1H), 11.08-11.19 (d, 1H). LCMS(ESI): [M]⁺ m/z: calcd 490.2; found 491.2; Rt=1.431 min.

Compound 27: Retention time: 37.52 min. ¹H NMR (600 MHZ, DMSO-d₆) δ (ppm) 1.03-1.06 (m, 3H), 1.33-1.41 (m, 1H), 1.69-1.93 (m, 2H), 2.09-2.33 (m, 8H), 2.77-2.89 (m, 2H), 3.22-3.25 (m, 3H), 3.48-4.05 (m, 1H), 5.29-5.71 (m, 1H), 7.01-7.10 (dd, 1H), 7.35-7.42 (dd, 1H), 7.45-7.50 (d, 1H), 7.81-7.89 (m, 2H), 8.02-8.06 (m, 2H), 8.41-8.50 (dd, 1H), 11.08-11.19 (d, 1H). LCMS(ESI): [M]⁺ m/z: calcd 490.2; found 491.2; Rt=1.422 min.

### Example 59. Synthesis of 2-Oxo-2-[(2R,5S)-5-methyl-2-[2-(1-methyl-4-piperidyl)-1,3-benzothiazol-5-yl]-1-piperidyl]-N-(1-tetrahydropyran-2-ylpyrazolo[4,3-c]pyridin-7-yl)acetamide (Compound 94)

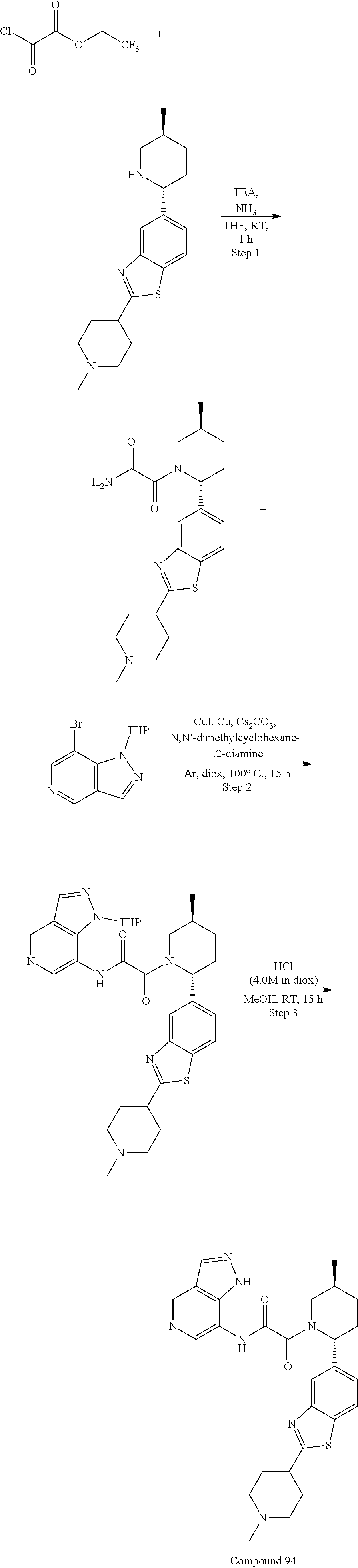

The synthesis of amine 5-((2R,5S)-5-methylpiperidin-2-yl)-2-(1-methylpiperidin-4-yl)benzo[d]thiazole is given in Intermediate 2.

### Step 1: The Synthesis of 2-((2R,5S)-5-methyl-2-(2-(1-methylpiperidin-4-yl)benzo[d]thiazol-5-yl)piperidin-1-yl)-2-oxoacetamide

Prepared by general procedure Scheme S5 step 1. Yield: 1.5 g (91.41%).

LCMS(ESI): [M+1]⁺ m/z: calcd 400.2; found 401.2; Rt=0.823 min.

### Step 2: The Synthesis of 2-oxo-2-[(2R,5S)-5-methyl-2-[2-(1-methyl-4-piperidyl)-1,3-benzothiazol-5-yl]-1-piperidyl]-N-(1-tetrahydropyran-2-ylpyrazolo[4,3-c]pyridin-7-yl)acetamide

Prepared by general procedure Scheme S5 step 2. Yield: 80.0 mg (31.32%). HPLC conditions: 0-5 min; 25-45% H₂O/MeCN/0.1% NH₄OH, flow: 30 ml/min (loading pump 4 ml/min acetonitrile) target mass 601 column: XBridge BEH C18 5 um 130 A.

LCMS(ESI): [M+1]⁺ m/z: calcd 601.2; found 602.2; Rt=2.045 min.

### Step 3: The Synthesis of 2-Oxo-2-[(2R,5S)-5-methyl-2-[2-(1-methyl-4-piperidyl)-1,3-benzothiazol-5-yl]-1-piperidyl]-N-(1-tetrahydropyran-2-ylpyrazolo[4,3-c]pyridin-7-yl)acetamide (Compound 94)

To the stirred solution of the 2-oxo-2-[(2R,5S)-5-methyl-2-[2-(1-methyl-4-piperidyl)-1,3-benzothiazol-5-yl]-1-piperidyl]-N-(1-tetrahydropyran-2-ylpyrazolo[4,3-c]pyridin-7-yl)acetamide (80 mg, 132.94 μmol) in MeOH (1 mL) Hydrogen chloride solution 4.0M in dioxane (400.00 mg, 10.97 mmol, 0.5 mL) was added. The resulting mixture was stirred at 25° C. for 15 hr. The reaction mixture was concentrated under reduce pressure. The residue was purified by reverse phase HPLC (30-80% 0-5 min H₂O/MeOH/0.1% NH₄OH, flow: 30 mL/min (loading pump 4 mL/min MeOH) column: XBridge C18 100×19 mm, 5 um) to give 2-oxo-N-(1H-pyrazolo[4,3-c]pyridin-7-yl)-2-[(2R,5S)-5-methyl-2-[2-(1-methyl-4-piperidyl)-1,3-benzothiazol-5-yl]-1-piperidyl]acetamide (39 mg, 75.34 μmol, 56.67% yield). ¹H NMR (600 MHz, DMSO-d₆) δ (ppm) 1.05 (m, 3H), 1.38 (dd, 1H), 1.81 (m, 5H), 2.04 (m, 5H), 2.18 (s, 3H), 2.81 (m, 2H), 3.00 (m, 2H), 3.94 (m, 1H), 5.67 (m, 1H), 7.41 (m, 1H), 7.91 (m, 1H), 8.05 (m, 1H), 8.33 (m, 1H), 8.47 (m, 1H), 8.93 (m, 1H), 11.09 (s, 1H), 13.00 (s, 1H).

LCMS(ESI): [M+2]⁺ m/z: calcd 519.2; found 519.2; Rt=1.865 min.

### Example 60. Synthesis of N-(8-amino-2,7-naphthyridin-4-yl)-2-((2R,5S)-2-(2-(3-((dimethylamino)methyl) oxetan-3-yl)benzo[d]thiazol-5-yl)-5-methylpiperidin-1-yl)-2-oxoacetamide (Compound 82)

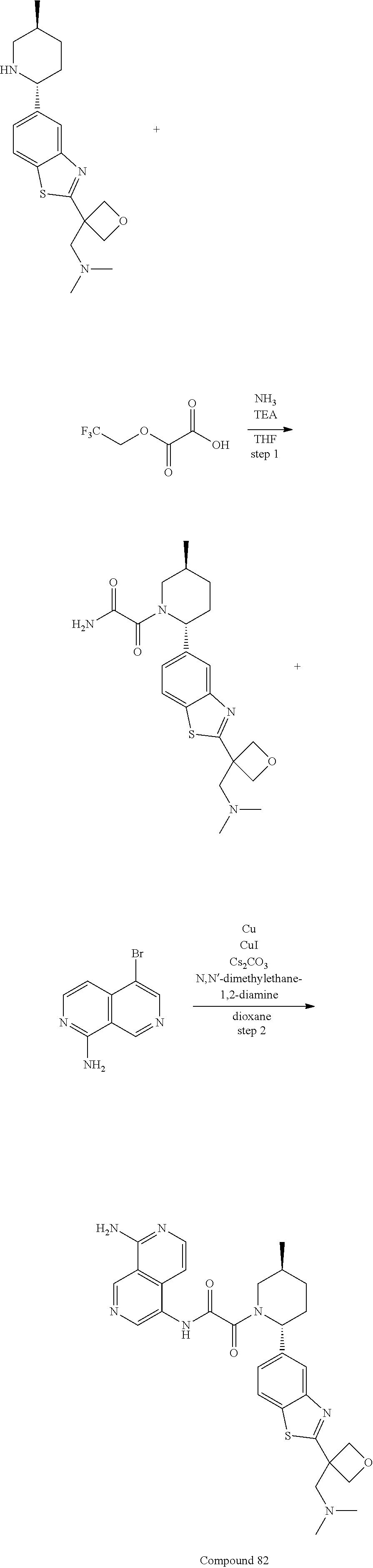 [SMILES: C[C@H]1CC[C@H](c2ccc3sc(C4(CN(C)C)COC4)nc3c2)NC1 ; O=C(O)C(=O)OCC(F)(F)F ; C[C@H]1CC[C@H](c2ccc3sc(C4(CN(C)C)COC4)nc3c2)N(C(=O)C(N)=O)C1 ; Nc1nccc2c(Br)cncc12 ; C[C@H]1CC[C@H](c2ccc3sc(C4(CN(C)C)COC4)nc3c2)N(C(=O)C(=O)Nc2cncc3c(N)nccc23)C1]

The synthesis of N,N-dimethyl-1-(3-(5-((2R,5S)-5-methylpiperidin-2-yl)benzo[d]thiazol-2-yl) oxetan-3-yl) methanamine is given in Intermediate 4.

### Step 1: Synthesis of 2-((2R,5S)-2-(2-(3-((dimethylamino)methyl) oxetan-3-yl)benzo[d]thiazol-5-yl)-5-methylpiperidin-1-yl)-2-oxoacetamide

2,2,2-Trifluoroethyl 2-chloro-2-oxo-acetate (143.36 mg, 752.53 μmol) was added slowly to a cooled to −10° C. solution of N,N-dimethyl-1-[3-[5-[(2R,5S)-5-methyl-2-piperidyl]-1,3-benzothiazol-2-yl]oxetan-3-yl]methanamine (200 mg, 578.87 μmol) and TEA (292.88 mg, 2.89 mmol, 403.41 μL) in THF (15 mL). The resulting mixture was allowed to warm to 25° C. and stirred for 2 hr. Gaseous ammonia (9.86 mg, 578.87 μmol) was vigorously bubbled through it at 25° C. for 1 hr. The resulting mixture was filtered to remove ammonium chloride; the filter cake was washed with THF (3*5 ml) and discarded. The combined filtrate was concentrated in vacuum to afford crude 2-oxo-2-[(2R,5S)-2-[2-[3-[(dimethylamino)methyl]oxetan-3-yl]-1,3-benzothiazol-5-yl]-5-methyl-1-piperidyl]acetamide (400 mg, crude) as yellow gum, which was used directly in the next step. LCMS(ESI): [M]⁺ m/z: calcd 416.2; found 417.2; Rt=2.099 min.

### Step 2: Synthesis of N-(8-amino-2,7-naphthyridin-4-yl)-2-((2R,5S)-2-(2-(3-((dimethylamino)methyl) oxetan-3-yl)benzo[d]thiazol-5-yl)-5-methylpiperidin-1-yl)-2-oxoacetamide (Compound 82)

A mixture of 2-oxo-2-[(2R,5S)-2-[2-[3-[(dimethylamino)methyl]oxetan-3-yl]-1,3-benzothiazol-5-yl]-5-methyl-1-piperidyl]acetamide (400 mg, 816.26 μmol), 5-bromo-2,7-naphthyridin-1-amine (219.47 mg, 979.51 μmol), copper (15 mg, 236.03 μmol), copper (I) iodide (31.09 mg, 163.25 μmol, 5.53 μL), cesium carbonate (664.88 mg, 2.04 mmol) and rac-(1R,2R)—N1,N2-dimethylcyclohexane-1,2-diamine (58.05 mg, 408.13 μmol) in dioxane (6 mL) was stirred under argon at 105° C. for 72 hr, then cooled down and diluted with DMSO (2 ml). The resulting mixture was submitted to reverse phase HPLC (column: YMC Triart C18 100×20 mm, 5 um; mobile phase: 45-90% 0-1-6 min H₂O/MeOH/0.1% NH₄OH, flow: 30 ml/min (loading pump 4 ml/min MeOH) to afford crude product (2 fractions, 96 mg, 58-65% purity by LCMS), which was reunified by reverse phase HPLC (column: YMC Triart C18 100×20 mm, 5 um; Mobile phase: 15-40% 0-6 min H₂O/MeCN/0.1% NH₄OH; flow rate: 30 mL/min (loading pump 4 ml/min MeOH)) to afford crude product 85% purity by LCMS (38 mg), which was reunified by preparative chiral HPLC (column: Chiralpak IA III (250*20 mm, 5 mkm); mobile phase: Hexane-IPA-MeOH, 50-25-25, flow rate: 12 ml/min) to afford Compound 82 (RetTime=58.484 min.)N-(8-amino-2,7-naphthyridin-4-yl)-2-oxo-2-[(2R,5S)-2-[2-[3-[(dimethylamino)methyl]oxetan-3-yl]-1,3-benzothiazol-5-yl]-5-methyl-1-piperidyl]acetamide (25 mg, 44.67 μmol, 5.47% yield) as beige solid.

Compound 82: ¹H NMR (DMSO-d6, 600 MHZ): δ (ppm) 1.10 (m, 3H), 1.38 (m, 1H), 1.74 (m, 1H), 1.91 (m, 1H), 2.24 (m, 6H), 2.92 (m, 2H), 3.81 (m, 3H), 4.80 (m, 2H), 4.93 (m, 2H), 5.54 (m, 1H), 6.86 (m, 1H), 7.40 (m, 3H), 7.91 (m, 1H), 8.06 (m, 1H), 8.13 (m, 1H), 8.70 (m, 1H), 9.38 (m, 1H), 10.94 (m, 1H). LCMS(ESI): [M]⁺ m/z: calcd 559.2; found 560.2; Rt=1.959 min.

### Example 61. Synthesis of N-(8-amino-2,7-naphthyridin-4-yl)-2-((2R,5S)-5-methyl-2-(2-(1-methylazetidin-3-yl)benzo[d]thiazol-5-yl)piperidin-1-yl)-2-oxoacetamide (Compound 53)

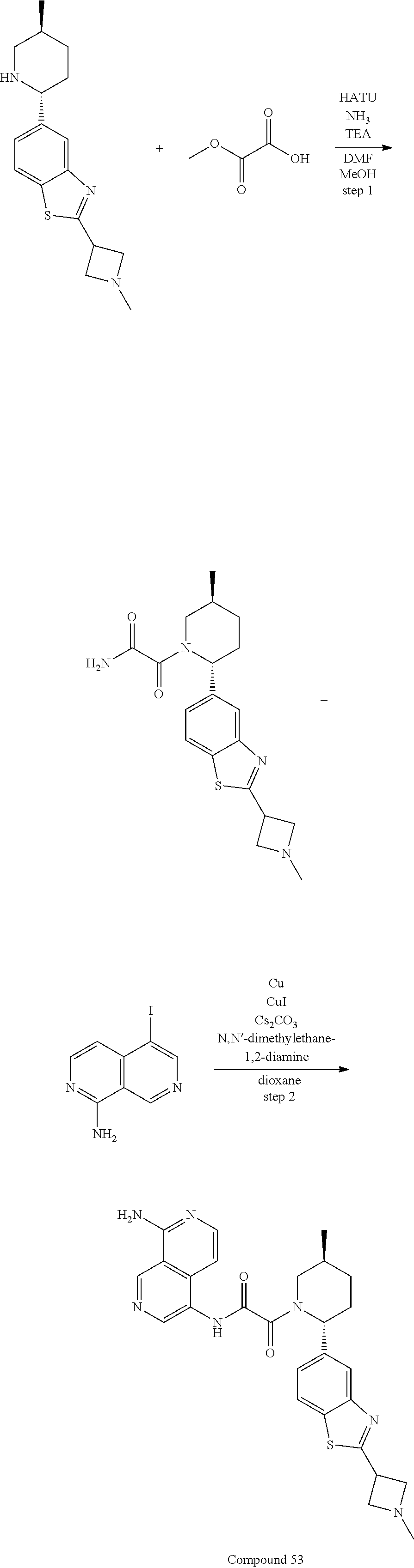

The synthesis of 2-(1-methylazetidin-3-yl)-5-((2R,5S)-5-methylpiperidin-2-yl)benzo[d]thiazole is given in Intermediate 6.

### Step 1: Synthesis of 2-((2R,5S)-5-methyl-2-(2-(1-methylazetidin-3-yl)benzo[d]thiazol-5-yl)piperidin-1-yl)-2-oxoacetamide

HATU (454.08 mg, 1.19 mmol) was added in small portions at 25° C. to a stirred mixture of 2-(1-methylazetidin-3-yl)-5-[(2R,5S)-5-methyl-2-piperidyl]-1,3-benzothiazole (300 mg, 995.19 μmol), 2-methoxy-2-oxo-acetic acid (155.34 mg, 1.49 mmol) and TEA (503.52 mg, 4.98 mmol, 693.55 μL) in DMF (4 mL). The resulting mixture was stirred at 25° C. for 2 hr. The LCMS indicated formation of desired amide-ester product (23% in reaction mixture). The reaction mixture was diluted with MeOH (15 mL) and gaseous ammonia was vigorously bubbled through it during 30 min. The LCMS showed only 1% of desired product in the reaction mixture. The reaction mixture was sealed tightly and allowed to stir at 25° C. for 15 hr. The LCMS showed 24% of desired product. MeOH was removed in vacuum, the resulting slurry was submitted to reverse phase HPLC (column: YMC Triart C18 100×20 mm, 5 um; mobile phase: 20-20-45% 0-1-4 min H₂O/MeCN/0.1% NH₄OH, flow rate: 30 ml/min; (loading pump 4 ml/min acetonitrile)) to afford 2-oxo-2-[(2R,5S)-5-methyl-2-[2-(1-methylazetidin-3-yl)-1,3-benzothiazol-5-yl]-1-piperidyl]acetamide (95 mg, 255.04 μmol, 25.63% yield) as light-brown gum. LCMS(ESI): [M]⁺ m/z: calcd 372.2; found 373.2; Rt=1.862 min.

### Step 2: Synthesis of N-(8-amino-2,7-naphthyridin-4-yl)-2-((2R,5S)-5-methyl-2-(2-(1-methylazetidin-3-yl)benzo[d]thiazol-5-yl)piperidin-1-yl)-2-oxoacetamide (Compound 53)

A mixture of 2-oxo-2-[(2R,5S)-5-methyl-2-[2-(1-methylazetidin-3-yl)-1,3-benzothiazol-5-yl]-1-piperidyl]acetamide (95 mg, 216.79 μmol), 5-iodo-2,7-naphthyridin-1-amine (64.64 mg, 238.47 μmol)(Intermediate 41), copper (10 mg, 157.36 μmol), copper (I) iodide (20 mg, 105.01 μmol, 3.56 μL), cesium carbonate (211.90 mg, 650.36 μmol) and (1R,2R)—N1,N2-dimethylcyclohexane-1,2-diamine (60 mg, 421.82 μmol) in dioxane (4.00 mL) was stirred under argon at 95° C. for 24 hr, then cooled down and filtered. The filter cake was washed with dioxane (2*1 ml) and discarded. The combined filtrate was submitted to reverse phase HPLC (column: XBridge C18 100×19 mm, 5 um; mobile phase: 20-70% 0-6 min H₂O/MeOH/0.1% NH₄OH, flow: 30 ml/min (loading pump 4 ml/min MeOH) to afford crude product (90 mg, 82% purity by LCMS), which was re-purified by reverse phase HPLC (column: Chromatorex 18 SMB100-5T 100×19 mm 5 um; Mobile phase: 0-0-25% 0-2-5 min H₂O/MeCN/0.1% FA; flow rate: 30 mL/min (loading pump 4 ml/min MeCN)) to afford pure Compound 53 N-(8-amino-2,7-naphthyridin-4-yl)-2-oxo-2-[(2R,5S)-5-methyl-2-[2-(1-methylazetidin-3-yl)-1,3-benzothiazol-5-yl]-1-piperidyl]acetamide (46.9 mg, 83.50 μmol, 38.52% yield, HCOOH) as yellow solid.

Compound 53: ¹H NMR (600 MHz, dmso) δ 1.05-1.13 (m, 3H), 1.35-1.43 (m, 1H), 1.70-1.77 (m, 1H), 1.85-1.96 (m, 1H), 2.11-2.24 (m, 1H), 2.28 (s, 3H), 2.31-2.35 (m, 1H), 2.90-2.95 (m, 1H), 3.65-3.70 (m, 3H), 4.01-4.12 (m, 2H), 5.27-5.79 (m, 1H), 6.61-7.00 (m, 1H), 7.28-7.38 (m, 2H), 7.39-7.49 (m, 1H), 7.91-7.98 (m, 1H), 8.01-8.36 (m, 3H), 8.61-8.77 (m, 1H), 9.31-9.45 (m, 1H), 10.80-11.02 (m, 1H). LCMS(ESI): [M]⁺ m/z: calcd 515.2; found 516.2; Rt=1.890 min.

### Example 62. Synthesis of 2-(2-(2-(2-(dimethylamino)ethyl)benzo[d]thiazol-5-yl)-5-methylpiperidin-1-yl)-2-oxo-N-(1H-pyrazolo[3,4-c]pyridin-4-yl)acetamide (Compound 92 and Compound 77)

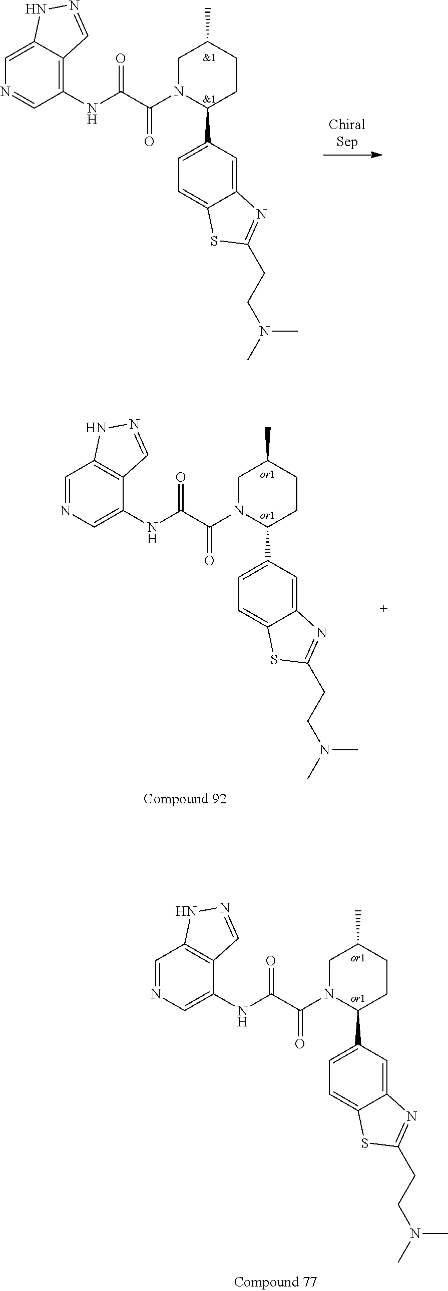 [SMILES: C[C@@H]1CC[C@@H](c2ccc3sc(CCN(C)C)nc3c2)N(C(=O)C(=O)Nc2cncc3[nH]ncc23)C1 ; C[C@H]1CC[C@H](c2ccc3sc(CCN(C)C)nc3c2)N(C(=O)C(=O)Nc2cncc3[nH]ncc23)C1 ; C[C@@H]1CC[C@@H](c2ccc3sc(CCN(C)C)nc3c2)N(C(=O)C(=O)Nc2cncc3[nH]ncc23)C1]

The compounds were prepared in accordance with general scheme S5, starting with step 2.A, using rac-2-((2R,5S)-2-(2-(2-(dimethylamino)ethyl)benzo[d]thiazol-5-yl)-5-methylpiperidin-1-yl)-2-oxoacetamide (synthesis given in Example 11).

### Chiral Separation (Compound 92 and Compound 77)

Racemic 2-[2-[2-[2-(dimethylamino)ethyl]-1,3-benzothiazol-5-yl]-5-methyl-1-piperidyl]-2-oxo-N-(1H-pyrazolo[3,4-c]pyridin-4-yl)acetamide (0.04 g, 81.37 μmol) was chiral separated (Column: ChiralArt YMC (250-20 mm-5 m); Mobile phase: IPA-MeOH, 50-50 Flow Rate: 10 mL/min) to obtain rel-2-((2R,5S)-2-(2-(2-(dimethylamino)ethyl)benzo[d]thiazol-5-yl)-5-methylpiperidin-1-yl)-2-oxo-N-(1H-pyrazolo[3,4-c]pyridin-4-yl)acetamide (15.10 mg, 30.72 μmol, 37.75% yield) and rel-2-((2R,5S)-2-(2-(2-(dimethylamino)ethyl)benzo[d]thiazol-5-yl)-5-methylpiperidin-1-yl)-2-oxo-N-(1H-pyrazolo[3,4-c]pyridin-4-yl)acetamide (0.05775 g, 124.60 μmol, 50.22% yield).

Rel Time for Compound 92 in analytical conditions (column: IC, IPA-MeOH, 50-50, 0.6 ml/min as mobile phase) 29.74 min and for Compound 5 22.52 min.

Compound 92: Retention time: 29.74 min. ¹H NMR (600 MHZ, DMSO-d₆) δ (ppm) 1.04-1.10 (m, 3H), 1.34-1.43 (m, 1H), 1.68-1.78 (m, 1H), 1.85-1.96 (m, 1H), 2.01-2.29 (m, 8H), 2.29-2.35 (m, 1H), 2.67-2.72 (m, 2H), 2.87-3.38 (m, 2H), 3.43-4.11 (m, 1H), 5.24-5.78 (m, 1H), 7.35-7.47 (m, 1H), 7.86-7.96 (m, 1H), 7.98-8.08 (m, 1H), 8.18-8.39 (m, 1H), 8.53-8.67 (m, 1H), 8.75-8.87 (m, 1H), 11.17-11.48 (m, 1H), 13.71 (s, 1H).

LCMS(ESI): [M]⁺ m/z: calcd 491.2; found 492.2; Rt=1.688 min.

Compound 77: Retention time: 22.52 min. ¹H NMR (600 MHz, DMSO-d₆) δ (ppm) 1.03-1.12 (m, 3H), 1.33-1.44 (m, 1H), 1.67-1.79 (m, 1H), 1.82-2.29 (m, 9H), 2.30-2.35 (m, 1H), 2.66-2.73 (m, 2H), 2.87-3.39 (m, 2H), 3.43-4.10 (m, 1H), 5.23-5.77 (m, 1H), 7.34-7.48 (m, 1H), 7.85-7.96 (m, 1H), 7.99-8.09 (m, 1H), 8.18-8.40 (m, 1H), 8.52-8.68 (m, 1H), 8.75-8.87 (m, 1H), 11.15-11.49 (m, 1H), 13.71 (s, 1H). LCMS(ESI): [M]⁺ m/z: calcd 491.2; found 492.2; Rt=1.689 min.

### Example 63. The synthesis of 2-((2R,5S)-2-(2-(1-ethylpiperidin-4-yl)benzo[d]thiazol-5-yl)-5-methylpiperidin-1-yl)-2-oxo-N-(1H-pyrazolo[4,3-c]pyridin-7-yl)acetamide (Compound 37)

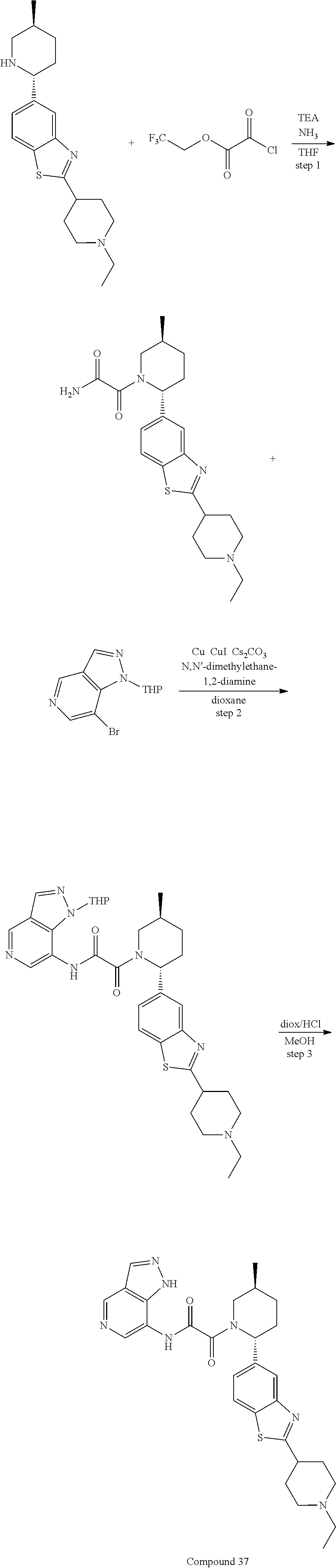

The synthesis of 2-(1-ethylpiperidin-4-yl)-5-((2R,5S)-5-methylpiperidin-2-yl)benzo[d]thiazole is given in Intermediate 25.

### Step 1: Synthesis of 2-((2R,5S)-2-(2-(1-ethylpiperidin-4-yl)benzo[d]thiazol-5-yl)-5-methylpiperidin-1-yl)-2-oxoacetamide

2,2,2-Trifluoroethyl 2-chloro-2-oxo-acetate (216.28 mg, 1.14 mmol) was slowly added to a stirred solution of 2-(1-ethyl-4-piperidyl)-5-[(2R,5S)-5-methyl-2-piperidyl]-1,3-benzothiazole (300 mg, 873.29 μmol) and TEA (220.92 mg, 2.18 mmol, 304.30 μL) in dry THF (20 mL) at 25° C. The resulting mixture was stirred at 25° C. for 1 hr, then gaseous ammonia was bubbled through the reaction mixture at 25° C. for 1 hr. The resulting ammonium chloride precipitate was filtered and discarded, the filtrate was concentrated in vacuum to afford crude 2-oxo-2-[(2R,5S)-2-[2-(1-ethyl-4-piperidyl)-1,3-benzothiazol-5-yl]-5-methyl-1-piperidyl]acetamide (250 mg, 603.04 μmol, 69.05% yield) as light-yellow gum, which was used directly in the next step. LCMS(ESI): [M]⁺ m/z: calcd 414.2; found 415.2; Rt=2.021 min.

### Step 2: Synthesis of 2-((2R,5S)-2-(2-(1-ethylpiperidin-4-yl)benzo[d]thiazol-5-yl)-5-methylpiperidin-1-yl)-2-oxo-N-(1-(tetrahydro-2H-pyran-2-yl)-1H-pyrazolo[4,3-c]pyridin-7-yl)acetamide

Prepared by general procedure Scheme S3 step 3A. Yield: 70 mg (18.85%).

HPLC conditions: Column: XBridge C18 100*19 mm, 5 microM; 0-5 min 40-90% water-MeOH+0.1% NH₄OH, flow: 30 ml/min; (loading pump 4 ml/min MeOH).

LCMS(ESI): [M]⁺ m/z: calcd 615.2; found 616.2; Rt=2.443 min.

### Step 3: Synthesis of 2-((2R,5S)-2-(2-(1-ethylpiperidin-4-yl)benzo[d]thiazol-5-yl)-5-methylpiperidin-1-yl)-2-oxo-N-(1H-pyrazolo[4,3-c]pyridin-7-yl)acetamide (Compound 37)

Hydrogen chloride, 4M in dioxane, 99% (787.50 mg, 3.00 mmol, 0.75 mL, 13.9% purity) was added in one portion to a stirred solution of 2-oxo-2-[(2R,5S)-2-[2-(1-ethyl-4-piperidyl)-1,3-benzothiazol-5-yl]-5-methyl-1-piperidyl]-N-(1-tetrahydropyran-2-ylpyrazolo[4,3-c]pyridin-7-yl)acetamide (70 mg, 113.68 μmol) in MeOH (2 mL) at 25° C. The resulting solution was stirred at 25° C. for 3 hr, and then evaporated to dryness in vacuum. The resulting light-yellow solid was submitted to reverse phase HPLC (column: YMC Triart C18 100×20 mm, 5 um; mobile phase: 30-80% 0-5 min H₂O/MeOH/0.1% NH₄OH, flow rate: 30 ml/min (loading pump 4 ml/min MeOH)) to afford Compound 37 2-oxo-N-(1H-pyrazolo[4,3-c]pyridin-7-yl)-2-[(2R,5S)-2-[2-(1-ethyl-4-piperidyl)-1,3-benzothiazol-5-yl]-5-methyl-1-piperidyl]acetamide (22.9 mg, 43.07 μmol, 37.89% yield) as light-yellow solid.

Compound 37: ¹H NMR (600 MHz, DMSO-d₆) δ (ppm) 1.00 (m, 3H), 1.06 (m, 3H), 1.38 (m, 1H), 1.82 (m, 4H), 2.07 (m, 5H), 2.34 (m, 3H), 2.93 (m, 3H), 3.08 (m, 1H), 3.95 (m, 1H), 5.68 (m, 1H), 7.41 (m, 1H), 7.91 (m, 1H), 8.05 (m, 1H), 8.33 (m, 1H), 8.47 (m, 1H), 8.93 (d, 1H), 11.13 (m, 1H), 13.07 (m, 1H). LCMS(ESI): [M]⁺ m/z: calcd 531.2; found 532.2; Rt=2.174 min.

### Example 64. Synthesis of rac-N-(4-amino-1H-pyrazolo[4,3-c]pyridin-7-yl)-2-((2R,5S)-2-(isoquinolin-6-yl)-5-methylpiperidin-1-yl)-2-oxoacetamide (Compound 22)

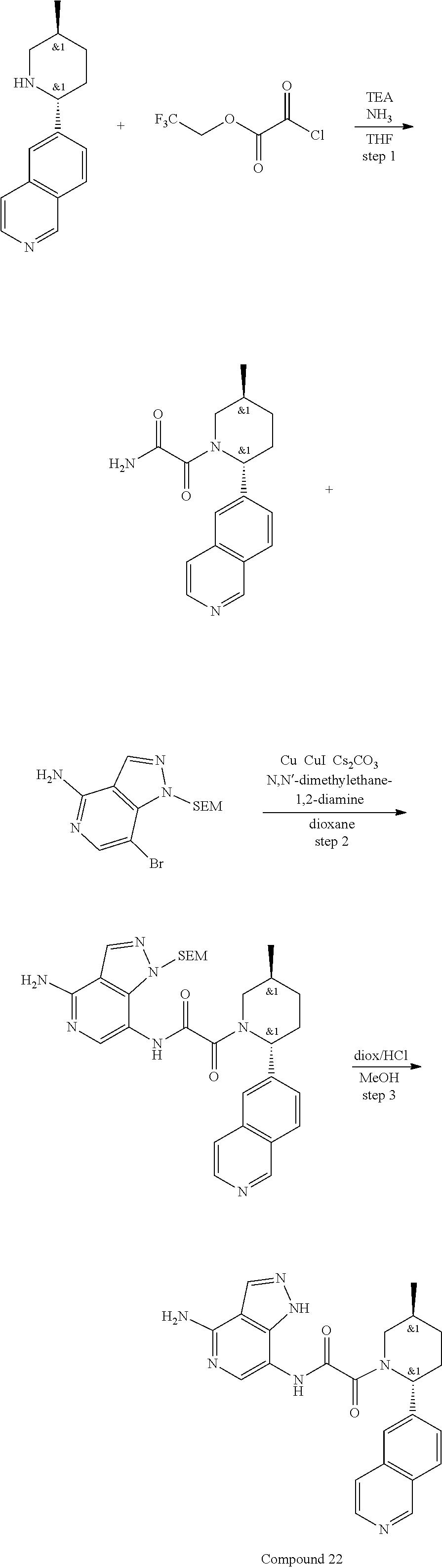 [SMILES: C[C@H]1CC[C@H](c2ccc3cnccc3c2)NC1 ; O=C(Cl)C(=O)OCC(F)(F)F ; C[C@H]1CC[C@H](c2ccc3cnccc3c2)N(C(=O)C(N)=O)C1 ; C[Si](C)(C)CCOCn1ncc2c(N)ncc(Br)c21 ; C[C@H]1CC[C@H](c2ccc3cnccc3c2)N(C(=O)C(=O)Nc2cnc(N)c3cnn(COCC[Si](C)(C)C)c23)C1 ; C[C@H]1CC[C@H](c2ccc3cnccc3c2)N(C(=O)C(=O)Nc2cnc(N)c3cn[nH]c23)C1]

### Step 1: Synthesis of rac-2-((2R,5S)-2-(isoquinolin-6-yl)-5-methylpiperidin-1-yl)-2-oxoacetamide

To a solution of 6-[rac-(2R,5S)-5-methyl-2-piperidyl]isoquinoline (0.23 g, 1.02 mmol) and TEA (205.67 mg, 2.03 mmol, 283.30 μL) in THF (4.90 mL) was added 2,2,2-trifluoroethyl 2-chloro-2-oxo-acetate (193.61 mg, 1.02 mmol, 129.07 μL) dropwise at 0° C. under argon. The reaction mixture was then stirred for 5 hr at rt, then was added ammonium (366.67 mg, 21.53 mmol). Resulting solution was stirred at 20° C. for 5 hr. Then, volatiles were removed under reduced pressure, leaving 2-oxo-2-[(2R,5S)-2-(6-isoquinolyl)-5-methyl-1-piperidyl]acetamide (250 mg, 840.76 μmol, 82.73% yield). LCMS(ESI): [M]⁺ m/z: calcd 297.2; found 298.2; Rt=0.816 min.

### Step 2: Synthesis of rac-N-(4-amino-1-((2-(trimethylsilyl)ethoxy)methyl)-1H-pyrazolo[4,3-c]pyridin-7-yl)-2-((2R,5S)-2-(isoquinolin-6-yl)-5-methylpiperidin-1-yl)-2-oxoacetamide

To an 8 ml vial 2-oxo-2-[rac-(2R,5S)-2-(6-isoquinolyl)-5-methyl-1-piperidyl]acetamide (120 mg, 403.56 μmol), 7-bromo-1-(2-trimethylsilylethoxymethyl)pyrazolo[4,3-c]pyridin-4-amine (138.54 mg, 403.56 μmol), copper (I) iodide (76.86 mg, 403.56 μmol, 13.68 μL), cesium carbonate (262.98 mg, 807.13 μmol), (1R,2R)—N1,N2-dimethylcyclohexane-1,2-diamine (86.10 mg, 605.35 μmol), copper (25.65 mg, 403.56 μmol) and dioxane (5.00 mL) were charged. The vial was purged with argon, sealed and heated at 90° C. (oil bath) overnight. The reaction mixture was filtered and the filtrate was concentrated in vacuum afforded crude N-[4-amino-1-(2-trimethylsilylethoxymethyl)pyrazolo[4,3-c]pyridin-7-yl]-2-oxo-2-[rac-(2R,5S)-2-(6-isoquinolyl)-5-methyl-1-piperidyl]acetamide (36.4 mg, 65.03 μmol, 16.11% yield) which was purified by HPLC (2-10 min 10-40% MeCN+FA 30 ml/min (loading pump 4 m MeCN) column: SunFire 100*19 mm, 5 microM). LCMS(ESI): [M]⁺ m/z: calcd 559.2; found 560.2; Rt=1.160 min.

### Step 2: Synthesis of rac-N-(4-amino-1H-pyrazolo[4,3-c]pyridin-7-yl)-2-((2R,5S)-2-(isoquinolin-6-yl)-5-methylpiperidin-1-yl)-2-oxoacetamide (Compound 22)

To a solution of N-[4-amino-1-(2-trimethylsilylethoxymethyl)pyrazolo[4,3-c]pyridin-7-yl]-2-oxo-2-[rac-(2R,5S)-2-(6-isoquinolyl)-5-methyl-1-piperidyl]acetamide (36.4 mg, 65.03 μmol) in MeOH (3 mL) was added hydrogen chloride solution 4.0M in dioxane (23.71 mg, 650.31 μmol, 29.64 μL) at 25° C. The resulting mixture was left to stir for 12 hr. The resulting mixture was evaporated to dryness and then submitted to reverse phase HPLC (2-10 min 35-50% MeOH+NH₃ flow 30 ml/min (loading pump 4 ml/min MeOH), Column Sun Fire C18 100*19 mm) to afford N-(4-amino-1H-pyrazolo[4,3-c]pyridin-7-yl)-2-oxo-2-[rac-(2R,5S)-2-(6-isoquinolyl)-5-methyl-1-piperidyl]acetamide (13 mg, 22.60 μmol, 34.75% yield, 4HCl).

Compound 22: ¹H NMR (600 MHZ, dmso) δ 0.96-1.10 (m, 3H), 1.27-1.47 (m, 1H), 1.71-1.79 (m, 1H), 1.86-2.00 (m, 1H), 2.13-2.35 (m, 1H), 2.74-3.06 (m, 1H), 3.49-4.16 (m, 2H), 5.18-5.82 (m, 1H), 6.56-7.09 (m, 2H), 7.54-7.96 (m, 4H), 8.05-8.25 (m, 2H), 8.41-8.53 (m, 1H), 9.13-9.33 (m, 1H), 9.62-10.67 (m, 1H), 12.63-13.40 (m, 1H).

LCMS(ESI): [M]⁺ m/z: calcd 429.2; found 430.2; Rt=0.585 min.

### Example 65. Synthesis of N-(4-amino-1H-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (Compound 105)

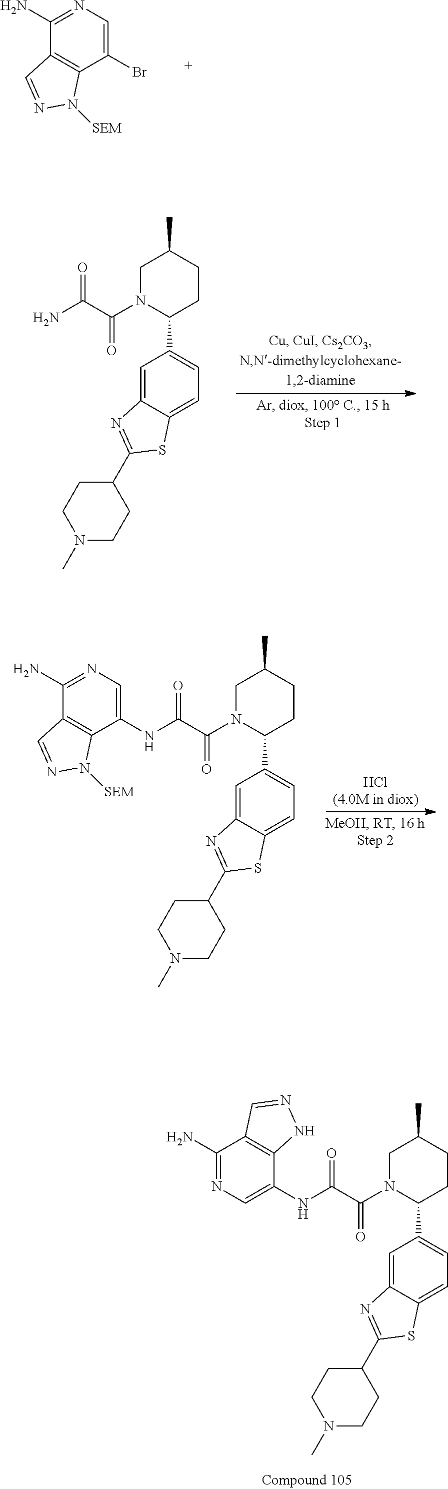 [SMILES: C[Si](C)(C)CCOCn1ncc2c(N)ncc(Br)c21 ; C[C@H]1CC[C@H](c2ccc3sc(C4CCN(C)CC4)nc3c2)N(C(=O)C(N)=O)C1 ; C[C@H]1CC[C@H](c2ccc3sc(C4CCN(C)CC4)nc3c2)N(C(=O)C(=O)Nc2cnc(N)c3cnn(COCC[Si](C)(C)C)c23)C1 ; C[C@H]1CC[C@H](c2ccc3sc(C4CCN(C)CC4)nc3c2)N(C(=O)C(=O)Nc2cnc(N)c3cn[nH]c23)C1]

The synthesis of 2-((2R,5S)-5-methyl-2-(2-(1-methylpiperidin-4-yl)benzo[d]thiazol-5-yl)piperidin-1-yl)-2-oxoacetamide is given in Intermediate 19.

### Step 1: The synthesis of N-[4-amino-1-(2-trimethylsilylethoxymethyl)pyrazolo[4,3-c]pyridin-7-yl]-2-oxo-2-[(2R,5S)-5-methyl-2-[2-(1-methyl-4-piperidyl)-1,3-benzothiazol-5-yl]-1-piperidyl]acetamide

Prepared by general procedure Scheme S3 step 3A. Yield: 23.0 mg (3.33%).

HPLC conditions: Column: 30-55% 0-5 min H₂O/MeCN/0.1% NH₄OH, flow: 30 ml/min (loading pump 4 ml/min ACN) target mass 662.94 column: XBridge C18 100×19 mm, 5 um.

LCMS(ESI): [M+1]⁺ m/z: calcd 663.2; found 663.2; Rt=2.489 min.

### Step 2: The synthesis of N-(4-amino-1H-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (Compound 105)

To the stirred solution of N-[4-amino-1-(2-trimethylsilylethoxymethyl)pyrazolo[4,3-c]pyridin-7-yl]-2-oxo-2-[(2R,5S)-5-methyl-2-[2-(1-methyl-4-piperidyl)-1,3-benzothiazol-5-yl]-1-piperidyl]acetamide (23 mg, 34.70 μmol) in MeOH (0.5 mL) Hydrogen chloride solution 4.0M in dioxane (400.00 mg, 10.97 mmol, 0.5 mL) was added. The resulting mixture was stirred at 25° C. for 3.5 hr. LCMS analysis showed 55% of starting material. The reaction mixture was then stirred at 25° C. for another 12 hr. The reaction mixture was concentrated under reduce pressure and was then purified by reverse phase HPLC (SYSTEM 40-40-90% 0-1-5 min H₂O/MeOH/0.1% NH₄OH, flow: 30 ml/min (loading pump 4 ml/min methanol) target mass 532 column: YMC Triart C18 100×20 mm, 5 um) to give N-(4-amino-1H-pyrazolo[4,3-c]pyridin-7-yl)-2-oxo-2-[(2R,5S)-5-methyl-2-[2-(1-methyl-4-piperidyl)-1,3-benzothiazol-5-yl]-1-piperidyl]acetamide (3 mg, 5.63 μmol, 16.23% yield).

¹H NMR (600 MHz, DMSO-d₆) δ (ppm) 0.99-1.10 (m, 3H), 1.33-1.42 (m, 1H), 1.66-1.75 (m, 1H), 1.86-1.95 (m, 1H), 2.06-2.15 (m, 2H), 2.26-2.34 (m, 3H), 2.71-2.91 (m, 4H), 3.05-3.15 (m, 2H), 3.42-4.11 (m, 5H), 5.41-5.76 (m, 1H), 7.42-7.50 (m, 1H), 7.89-8.02 (m, 2H), 8.05-8.13 (m, 1H), 8.48-10.48 (m, 3H), 10.90-11.23 (m, 1H), 12.22-14.15 (m, 1H). LCMS(ESI): [M+1]⁻ m/z: calcd 531.2; found 531.2; Rt=2.155 min.

### Example 66. Synthesis of N-(4-amino-1H-pyrazolo[4,3-c]pyridin-7-yl)-2-((2R,5S)-5-methyl-2-(3-(4-methylpiperazin-1-yl)phenyl)piperidin-1-yl)-2-oxoacetamide (Compound 25)

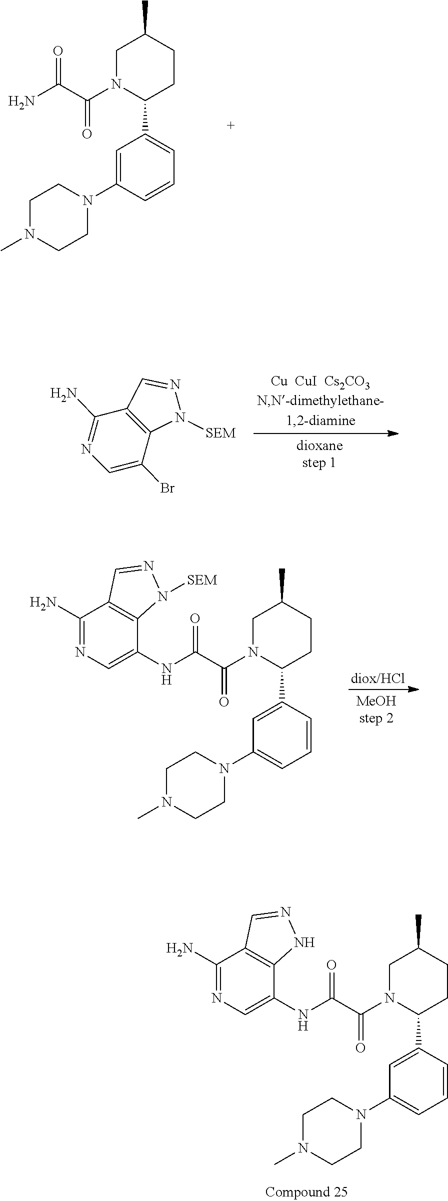 [SMILES: C[C@H]1CC[C@H](c2cccc(N3CCN(C)CC3)c2)N(C(=O)C(N)=O)C1 ; C[Si](C)(C)CCOCn1ncc2c(N)ncc(Br)c21 ; C[C@H]1CC[C@H](c2cccc(N3CCN(C)CC3)c2)N(C(=O)C(=O)Nc2cnc(N)c3cnn(COCC[Si](C)(C)C)c23)C1 ; C[C@H]1CC[C@H](c2cccc(N3CCN(C)CC3)c2)N(C(=O)C(=O)Nc2cnc(N)c3cn[nH]c23)C1]

The synthesis of 2-((2R,5S)-5-methyl-2-(3-(4-methylpiperazin-1-yl)phenyl)piperidin-1-yl)-2-oxoacetamide is given in Intermediate 28.

### Step 1: Synthesis of N-(4-amino-1-((2-(trimethylsilyl)ethoxy)methyl)-1H-pyrazolo[4,3-c]pyridin-7-yl)-2-((2R,5S)-5-methyl-2-(3-(4-methylpiperazin-1-yl)phenyl)piperidin-1-yl)-2-oxoacetamide

2-Oxo-2-[(2R,5S)-5-methyl-2-[3-(4-methylpiperazin-1-yl)phenyl]-1-piperidyl]acetamide (170 mg, 493.54 μmol), 7-bromo-1-(2-trimethylsilylethoxymethyl)pyrazolo[4,3-c]pyridin-4-amine (169.43 mg, 493.54 μmol), copper (I) iodide (93.99 mg, 493.54 μmol, 16.73 μL), cesium carbonate (321.61 mg, 987.08 μmol) and (1R,2R)—N1,N2-dimethylcyclohexane-1,2-diamine (105.30 mg, 740.31 μmol) were mixed in dioxane (5 mL) under argon, and then stirred overnight at 100° C. for 16 hr in vial. The reaction mixture was filtered and the filtrate was concentrated in vacuum and purified by HPLC (SYSTEM 2-10 min 25-50% MeOH+FA flow 30 ml/min (loading pump 4 ml/min MeOH), Column Sun Fire C18 100*19 mm) to give N-[4-amino-1-(2-trimethylsilylethoxymethyl)pyrazolo[4,3-c]pyridin-7-yl]-2-oxo-2-[(2R,5S)-5-methyl-2-[3-(4-methylpiperazin-1-yl)phenyl]-1-piperidyl]acetamide (69 mg, 113.71 μmol, 23.04% yield).

LCMS(ESI): [M]⁺ m/z: calcd 606.2; found 607.2; Rt=0.805 min.

### Step 2: Synthesis of N-(4-amino-1H-pyrazolo[4,3-c]pyridin-7-yl)-2-((2R,5S)-5-methyl-2-(3-(4-methylpiperazin-1-yl)phenyl)piperidin-1-yl)-2-oxoacetamide (Compound 25)

To a solution of N-[4-amino-1-(2-trimethylsilylethoxymethyl)pyrazolo[4,3-c]pyridin-7-yl]-2-oxo-2-[(2R,5S)-5-methyl-2-[3-(4-methylpiperazin-1-yl)phenyl]-1-piperidyl]acetamide (69 mg, 113.71 μmol) in MeOH (5.00 mL) was added hydrogen chloride solution 4.0M in dioxane (800.00 mg, 21.94 mmol, 1.00 mL) at 26° C. The resulting mixture was left to stir for 14 hr. The resulting mixture was evaporated to dryness and then submitted to reverse phase HPLC (SYSTEM 2-10 min 0-100% MeOH+FA flow 30 ml/min (loading pump 4 ml MeOH), column: sun fire c18) to afford N-(4-amino-1H-pyrazolo[4,3-c]pyridin-7-yl)-2-oxo-2-[(2R,5S)-5-methyl-2-[3-(4-methylpiperazin-1-yl)phenyl]-1-piperidyl]acetamide (23.7 mg, 42.39 μmol, 37.28% yield, HCl—HCOOH).

Compound 25: ¹H NMR (600 MHZ, DMSO-d₆) δ (ppm) 1.02-1.07 (m, 3H), 1.27-1.41 (m, 1H), 1.61-1.76 (m, 1H), 1.78-2.08 (m, 2H), 2.14-2.28 (m, 5H), 2.73-2.81 (m, 1H), 3.03-3.09 (m, 3H), 3.10-3.15 (m, 4H), 3.70-4.08 (m, 1H), 4.90-5.61 (m, 1H), 6.61-6.70 (m, 2H), 6.73-6.85 (m, 3H), 7.14-7.25 (m, 1H), 7.48-7.75 (m, 1H), 8.16-8.24 (m, 1H), 10.41-10.55 (m, 1H), 12.61-12.79 (m, 1H). LCMS(ESI): [M]⁺ m/z: calcd 476.2; found 477.2; Rt=0.723 min.

### Example 67. Synthesis of 2-[(2R,5S)-5-methyl-2-(2-tetrahydropyran-4-yl-1,3-benzothiazol-5-yl)-1-piperidyl]-2-oxo-N-(1H-pyrazolo[4,3-c]pyridin-7-yl)acetamide (Compound 97)

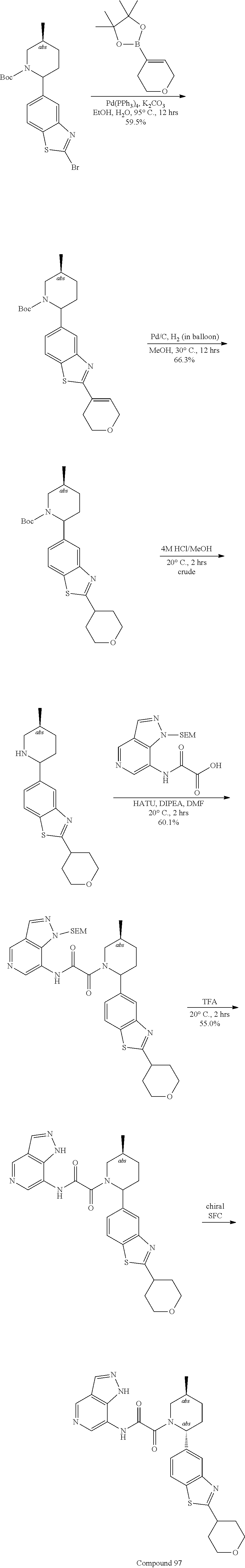

### Step 1: Synthesis of tert-butyl (5S)-2-[2-(3,6-dihydro-2H-pyran-4-yl)-1,3-benzothiazol-5-yl]-5-methyl-piperidine-1-carboxylate

A mixture of tert-butyl (5S)-2-(2-bromo-1,3-benzothiazol-5-yl)-5-methyl-piperidine-1-carboxylate (500 mg, 1.22 mmol), 2-(3,6-dihydro-2H-pyran-4-yl)-4,4,5,5-tetramethyl-1,3,2-dioxaborolane (350 mg, 1.67 mmol), K₂CO₃ (500 mg, 3.62 mmol), palladium; triphenylphosphane (150 mg, 0.130 mmol), EtOH (10 mL) and H₂O (3 mL) was stirred at 95° C. for 12 hours. The resulting mixture was quenched by addition of water (10 mL) and extracted with EtOAc (20 mL*3). The combined organic layer was 10 washed with saturated NH₄Cl aqueous solution (10 mL*2), brine (10 mL), dried over anhydrous Na₂SO₄, filtered and concentrated under reduced pressure. The residue was purified by flash chromatography (ISCO®; 24 g AgelaFlash® Silica Flash Column, petroleum ether/EtOAc with EtOAc from 0˜40%, Flow Rate: 30 mL/min) to afford tert-butyl (5S)-2-[2-(3,6-dihydro-2H-pyran-4-yl)-1,3-benzothiazol-5-yl]-5-methyl-piperidine-1-carboxylate (300 mg, 59.5% yield) as yellow solid. ¹H NMR (400 MHZ, methanol-d₄) δ ppm 7.93 (d, J=8.4 Hz, 1H), 7.78-7.86 (m, 1H), 7.31-7.39 (m, 1H), 6.85 (dt, J=3.0, 1.5 Hz, 1H), 5.40 (t, J=4.8 Hz, 1H), 4.38 (q, J=2.8 Hz, 2H), 3.96 (t, J=5.5 Hz, 2H), 3.79 (br d, J=13.5 Hz, 1H), 3.14 (dd, J=13.5, 3.9 Hz, 1H), 2.73-2.82 (m, 2H), 2.13-2.27 (m, 2H), 1.78-1.97 (m, 2H), 1.41-1.54 (m, 9H), 1.33-1.40 (m, 1H), 1.10 (d, J=6.9 Hz, ³H);

LCMS(ESI) [M+H]⁺ m/z: calcd 415.2; found 415.2.

### Step 2: Synthesis of tert-butyl (5S)-5-methyl-2-(2-tetrahydropyran-4-yl-1,3-benzothiazol-5-yl)piperidine-1-carboxylate

A mixture of tert-butyl (5S)-2-[2-(3,6-dihydro-2H-pyran-4-yl)-1,3-benzothiazol-5-yl]-5-methyl-piperidine-1-carboxylate (300 mg, 0.724 mmol), Pd/C (200 mg, 10% of Pd with 50% of water, wt %) and MeOH (5 mL) was stirred at 30° C. for 12 hours under H₂ atmosphere (in balloon). The mixture was filtered and concentrated under reduced pressure to give tert-butyl (5S)-5-methyl-2-(2-tetrahydropyran-4-yl-1,3-benzothiazol-5-yl)piperidine-1-carboxylate (200 mg, 66.3% yield) as yellow solid.

### Step 3: Synthesis of 5-[(5S)-5-methyl-2-piperidyl]-2-tetrahydropyran-4-yl-1,3-benzothiazole)

A mixture of tert-butyl (5S)-5-methyl-2-(2-tetrahydropyran-4-yl-1,3-benzothiazol-5-yl)piperidine-1-carboxylate (180 mg, 0.432 mmol), 4M HCl/MeOH (10 mL, 40.0 mmol) was stirred at 20° C. for 2 hours. The mixture was filtered and concentrated under reduced pressure to give 5-[(5S)-5-methyl-2-piperidyl]-2-tetrahydropyran-4-yl-1,3-benzothiazole (150 mg, crude) as yellow oil.

### Step 4: Synthesis of 2-[(5S)-5-methyl-2-(2-tetrahydropyran-4-yl-1,3-benzothiazol-5-yl)-1-piperidyl]-2-oxo-N-[1-(2-trimethylsilylethoxymethyl)pyrazolo[4,3-c]pyridin-7-yl]acetamide

To a mixture of 2-oxo-2-[[1-(2-trimethylsilylethoxymethyl)pyrazolo[4,3-c]pyridin-7-yl]amino]acetic acid (Intermediate 37)(150 mg, 0.446 mmol) and 5-[(5S)-5-methyl-2-piperidyl]-2-tetrahydropyran-4-yl-1,3-benzothiazole (160 mg, 0.506 mmol) in DMF (2 mL) were added HATU (210. mg, 0.552 mmol) and DIPEA (0.5 mL, 2.87 mmol). The resulting solution was stirred at 20° C. for 2 hours. The resulting mixture was quenched by addition of water (10 mL) and extracted with EtOAc (50 mL*3). The combined organic layer was washed with saturated NH₄Cl aqueous solution (10 mL*2), brine (10 mL), dried over anhydrous Na₂SO₄, filtered and concentrated under reduced pressure to give 2-[(5S)-5-methyl-2-(2-tetrahydropyran-4-yl-1,3-benzothiazol-5-yl)-1-piperidyl]-2-oxo-N-[1-(2-trimethylsilylethoxymethyl)pyrazolo[4,3-c]pyridin-7-yl]acetamide (170 mg, 60.1% yield) as yellow oil. LCMS(ESI) [M+H]⁺ m/z: calcd 635.3; found 635.3.

### Step 5: Synthesis of 2-[(5S)-5-methyl-2-(2-tetrahydropyran-4-yl-1,3-benzothiazol-5-yl)-1-piperidyl]-2-oxo-N-(1H-pyrazolo[4,3-c]pyridin-7-yl)acetamide

A mixture of 2-[(5S)-5-methyl-2-(2-tetrahydropyran-4-yl-1,3-benzothiazol-5-yl)-1-piperidyl]-2-oxo-N-[1-(2-trimethylsilylethoxymethyl)pyrazolo[4,3-c]pyridin-7-yl]acetamide (160 mg, 0.252 mmol), TFA (3 mL, 38.9 mmol) was stirred at 20° C. for 2 hours. The mixture was concentrated under reduced pressure. The residue was added NH₃—H₂O (2 ml) and purified by flash chromatography (Column: SepaFlash®Sphercial C18, 25 g, 40-60 μm, 120 Å; MeCN/water (0.5% NH₃—H₂O) with MeCN from 0-50%, 25 mL/min, 220 nm) to give 2-[(5S)-5-methyl-2-(2-tetrahydropyran-4-yl-1,3-benzothiazol-5-yl)-1-piperidyl]-2-oxo-N-(1H-pyrazolo[4,3-c]pyridin-7-yl)acetamide (70 mg, 55.0% yield) as yellow oil. LCMS(ESI) [M+H]⁺ m/z: calcd 505.2, found 505.2.

### Step 6: Synthesis of 2-[(2R,5S)-5-methyl-2-(2-tetrahydropyran-4-yl-1,3-benzothiazol-5-yl)-1-piperidyl]-2-oxo-N-(1H-pyrazolo[4,3-c]pyridin-7-yl)acetamide (Compound 97)

2-[(5S)-5-methyl-2-(2-tetrahydropyran-4-yl-1,3-benzothiazol-5-yl)-1-piperidyl]-2-oxo-N-(1H-pyrazolo[4,3-c]pyridin-7-yl)acetamide (70 mg, 0.139 mmol) was purified by SFC (Instrument: Berger, Multigr AM-II; Column: Daicel Chiralpak AD 250 mm×30 mm×10 μm; Mobile phase: supercritical CO₂/EtOH (0.1% NH₃—H₂O, v %)=60/40; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to Compound 97.

Compound 97:2-[(2R,5S)-5-methyl-2-(2-tetrahydropyran-4-yl-1,3-benzothiazol-5-yl)-1-piperidyl]-2-oxo-N-(1H-pyrazolo[4,3-c]pyridin-7-yl)acetamide (42 mg, single known enantiomer with trans relative chemistry, peak 2, retention time=1.699 min, white solid). 1H NMR (400 MHZ, methanol-d₄) δ ppm 8.75-8.94 (m, 1H), 8.13-8.39 (m, 2H), 7.79-7.93 (m, 2H), 7.37 (br d, J=7.0 Hz, 1H), 5.38-5.82 (m, 1H), 3.81-4.05 (m, 3H), 3.51 (br t, J=11.5 Hz, 2H), 3.24-3.42 (m, 2H), 2.26 (br s, 2H), 1.76-2.03 (m, 6H), 1.39 (br d, J=12.0 Hz, 1H), 1.07 (br d, J=6.9 Hz, ³H); LCMS(ESI) [M+H]⁺ m/z: calcd 505.2, found 505.2; HPLC: 99.08%@220 nm, 100%@254 nm; 100% ee.

### Example 68. Synthesis of 2-oxo-N-(1H-pyrazolo[4,3-c]pyridin-7-yl)-2-[(5S)-5-methyl-2-[2-(4-piperidyl)-1,3-benzothiazol-5-yl]-1-piperidyl]acetamide (Compound 6)

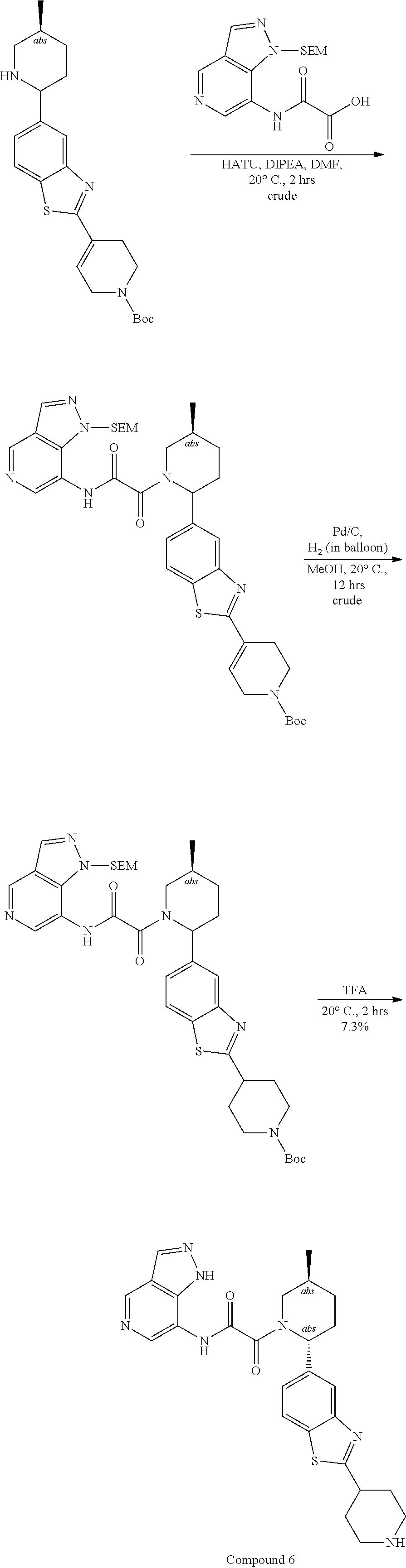 [SMILES: C[C@H]1CCC(c2ccc3sc(C4=CCN(C(=O)OC(C)(C)C)CC4)nc3c2)NC1 ; C[Si](C)(C)CCOCn1ncc2cncc(NC(=O)C(=O)O)c21 ; C[C@H]1CCC(c2ccc3sc(C4=CCN(C(=O)OC(C)(C)C)CC4)nc3c2)N(C(=O)C(=O)Nc2cncc3cnn(COCC[Si](C)(C)C)c23)C1 ; C[C@H]1CCC(c2ccc3sc(C4CCN(C(=O)OC(C)(C)C)CC4)nc3c2)N(C(=O)C(=O)Nc2cncc3cnn(COCC[Si](C)(C)C)c23)C1 ; C[C@H]1CC[C@H](c2ccc3sc(C4CCNCC4)nc3c2)N(C(=O)C(=O)Nc2cncc3cn[nH]c23)C1]

### Step 1: Synthesis of tert-butyl 4-[5-[(5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]-3,6-dihydro-2H-pyridine-1-carboxylate

To a solution of 2-oxo-2-[[1-(2-trimethylsilylethoxymethyl)pyrazolo[4,3-c]pyridin-7-yl]amino]acetic acid (Intermediate 37)(200 mg, 0.595 mmol), tert-butyl 4-[5-[(5S)-5-methyl-2-piperidyl]-1,3-benzothiazol-2-yl]-3,6-dihydro-2H-pyridine-1-carboxylate (248 mg, 0.600 mmol) and HATU (272 mg, 0.715 mmol) in DCM (10 mL) was added DIPEA (0.31 mL, 1.78 mmol). The mixture was stirred at 20° C. for 1 hour. The mixture was concentrated under reduced pressure. The residue was purified by flash chromatography (ISCO®; 12 g AgelaFlash® Silica Flash Column, petroleum ether/EtOAc with EtOAc from 0˜65%, 30 mL/min, 254 nm) to afford tert-butyl 4-[5-[(5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]-3,6-dihydro-2H-pyridine-1-carboxylate (400 mg, crude) as black oil. LCMS(ESI) [M+H]⁺ m/z: calcd 732.3; found 732.4.

### Step 2: Synthesis of tert-butyl 4-[5-[(5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]piperidine-1-carboxylate

To a solution of tert-butyl 4-[5-[(5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]-3,6-dihydro-2H-pyridine-1-carboxylate (400 mg, 0.546 mmol) in MeOH (20 mL) was added Pd—C(400 mg, 10% of Pd with 50% of water, wt %) under N₂ atmosphere. The suspension was degassed and purged with hydrogen for 3 times. The mixture was stirred under hydrogen (in balloon) at 20° C. for 12 hours. The mixture was filtered. The filtrate was concentrated under reduced pressure to give tert-butyl 4-[5-[(5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]piperidine-1-carboxylate (400 mg, crude) as brown oil, which was directly used without further purification. LCMS(ESI) [M+H]⁺ m/z: calcd 734.3; found 734.3.

### Step 3: Synthesis of 2-oxo-N-(1H-pyrazolo[4,3-c]pyridin-7-yl)-2-[(5S)-5-methyl-2-[2-(4-piperidyl)-1,3-benzothiazol-5-yl]-1-piperidyl]acetamide (Compound 6)

A mixture of tert-butyl 4-[5-[(5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]piperidine-1-carboxylate (400 mg, 0.545 mmol) in TFA (3 mL, 38.9 mmol) was stirred at 20° C. for 2 hours. The mixture was concentrated under reduced pressure. The residue was dissolved with H₂O (20 mL), and extracted with DCM (15 mL*3). The combined organic layer was dried over anhydrous Na₂SO₄, filtered and concentrated under reduced pressure. The residue was purified by preparative HPLC (Instrument: Gilson GX-281 Liquid Handler, Gilson 322 Pump, Gilson 156 UV Detector; Column: Phenomenex Gemini-NX 80×40 mm×3 μm; Mobile phase A: H₂O with 10 mmol NH₄HCO₃ (v %); Mobile phase B: MeCN; Gradient: B from 22% to 52% in 9.5 min, hold 100% B for 2 min; Flow Rate: 25 mL/min; Column Temperature: 30° C.; Wavelength: 220 nm, 254 nm) to give 2-oxo-N-(1H-pyrazolo[4,3-c]pyridin-7-yl)-2-[(5S)-5-methyl-2-[2-(4-piperidyl)-1,3-benzothiazol-5-yl]-1-piperidyl]acetamide (20 mg, 7.3% yield) as white dry powder. ¹H NMR (400 MHz, methanol-d₄) δ ppm 8.85-9.06 (m, 1H), 8.21-8.50 (m, 2H), 7.87-8.04 (m, 2H), 7.48 (d, J=7.0 Hz, 1H), 5.63-5.99 (m, 1H), 3.92-4.20 (m, 1H), 3.50 (d, J=14.6 Hz, 1H), 3.26 (d, J=12.8 Hz, 2H), 2.89 (t, J=11.8 Hz, 2H), 2.37 (s, 2H), 2.16-2.27 (m, 2H), 1.84-2.00 (m, 4H), 1.26-1.54 (m, 2H), 1.11-1.21 (m, ³H); LCMS(ESI) [M+H]⁺ m/z: calcd 504.2; found 504.3; HPLC: 90.30%@220 nm, 95.36%@254 nm; 94.8% ee.

### Example 69. Synthesis of N-imidazo[1,2-a]pyridin-7-yl-2-oxo-2-[(2R,5S)-5-methyl-2-[2-[rel-(3S,4R)-1,3-dimethyl-4-piperidyl]-1,3-benzothiazol-5-yl]-1-piperidyl]acetamide (Compound 23)

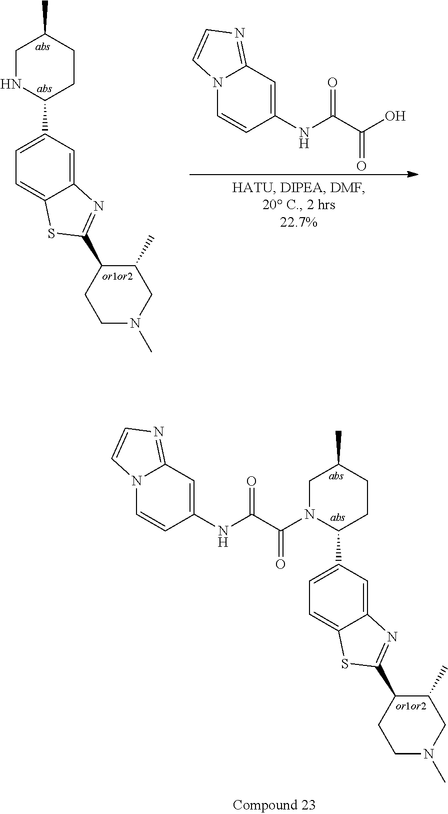 [SMILES: C[C@H]1CC[C@H](c2ccc3sc([C@@H]4CCN(C)C[C@H]4C)nc3c2)NC1 ; O=C(O)C(=O)Nc1ccn2ccnc2c1 ; C[C@H]1CC[C@H](c2ccc3sc([C@@H]4CCN(C)C[C@H]4C)nc3c2)N(C(=O)C(=O)Nc2ccn3ccnc3c2)C1]

### Step 1: Synthesis of N-imidazo[1,2-a]pyridin-7-yl-2-oxo-2-[(2R,5S)-5-methyl-2-[2-[rel-(3S,4R)-1,3-dimethyl-4-piperidyl]-1,3-benzothiazol-5-yl]-1-piperidyl]acetamide (Compound 23)

To a solution of 2-[rel-(3S,4R)-1,3-dimethyl-4-piperidyl]-5-[(2R,5S)-5-methyl-2-piperidyl]-1,3-benzothiazole (70 mg, 0.204 mmol), 2-(imidazo[1,2-a]pyridin-7-ylamino)-2-oxo-acetic acid (50 mg, 0.244 mmol) in DMF (3 mL) was added HATU (96 mg, 0.252 mmol) and DIPEA (0.112 mL, 0.643 mmol). The mixture was stirred at 20° C. for 2 hours. The resulting mixture was adjusted to pH=8 with NH₃—H₂O (12 N), then the mixture was concentrated under reduced pressure to give a crude product, which was purified by preparative HPLC (Instrument: Gilson GX-281 Liquid Handler, Gilson 322 Pump, Gilson 156 UV Detector; Column: 2_Phenomenex Gemini C18 75*40 mm*3 μm; Mobile phase A: H₂O with 0.05% NH₃—H₂O (v %); Mobile phase B: MeCN; Gradient: B from 35% to 65% in 9.5 min, hold 100% B for 2 min; Flow Rate: 25 mL/min; Column Temperature: 30° C.; Wavelength: 220 nm, 254 nm) to afford N-imidazo[1,2-a]pyridin-7-yl-2-oxo-2-[(2R,5S)-5-methyl-2-[2-[rel-(3S,4R)-1,3-dimethyl-4-piperidyl]-1,3-benzothiazol-5-yl]-1-piperidyl]acetamide (24.5 mg, single unknown enantiomer with trans relative chemistry, white solid).

Compound 23: ¹H NMR (400 MHz, methanol-d₄) δ ppm 8.25-8.47 (m, 1H), 7.89-8.16 (m, 3H), 7.71-7.85 (m, 1H), 7.43-7.55 (m, 2H), 6.99-7.20 (m, 1H), 5.44-5.86 (m, 1H), 3.75-4.05 (m, 1H), 3.47 (br d, J=13.1 Hz, 1H), 2.95-3.10 (m, 2H), 2.69-2.85 (m, 1H), 2.35 (br s, 5H), 1.80-2.23 (m, 7H), 1.41-1.53 (m, 1H), 1.41-1.53 (m, 1H), 1.50 (br s, 1H), 1.15 (d, J=6.9 Hz, ³H), 0.77-0.91 (m, ³H); LCMS(ESI) [M+H]⁺ m/z: calcd 531.3, found 531.2; HPLC: 100%@254 nm; SFC: 100% ee.

### Example 70. Synthesis of N-imidazo[1,2-a]pyridin-7-yl-2-oxo-2-[(2R,5S)-5-methyl-2-[2-[rel-(3S,4S)-1,3-dimethyl-4-piperidyl]-1,3-benzothiazol-5-yl]-1-piperidyl]acetamide (Compound 58)

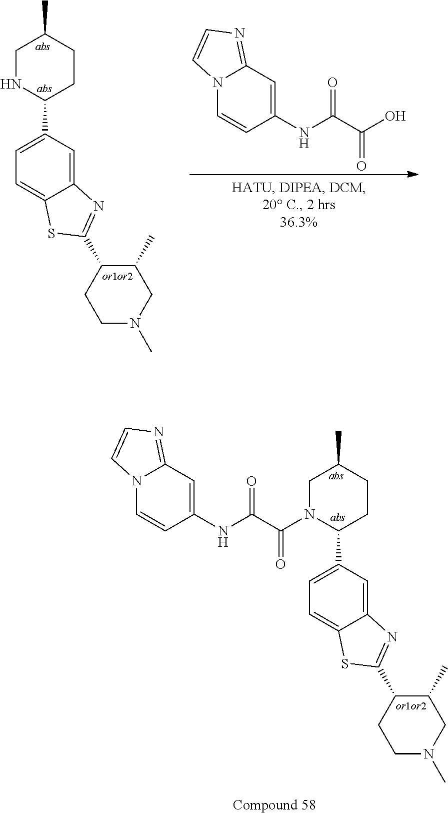 [SMILES: C[C@H]1CC[C@H](c2ccc3sc([C@H]4CCN(C)C[C@H]4C)nc3c2)NC1 ; O=C(O)C(=O)Nc1ccn2ccnc2c1 ; C[C@H]1CC[C@H](c2ccc3sc([C@H]4CCN(C)C[C@H]4C)nc3c2)N(C(=O)C(=O)Nc2ccn3ccnc3c2)C1]

### Step 1: Synthesis of N-imidazo[1,2-a]pyridin-7-yl-2-oxo-2-[(2R,5S)-5-methyl-2-[2-[rel-(3S,4S)-1,3-dimethyl-4-piperidyl]-1,3-benzothiazol-5-yl]-1-piperidyl]acetamide (Compound 58)

To a solution of 2-[rel-(3S,4S)-1,3-dimethyl-4-piperidyl]-5-[(2R,5S)-5-methyl-2-piperidyl]-1,3-benzothiazole (50 mg, 0.146 mmol), 2-(imidazo[1,2-a]pyridin-7-ylamino)-2-oxo-acetic acid (35 mg, 0.171 mmol), HATU (70 mg, 0.184 mmol) in DMF (4 mL) was added N-ethyl-N-isopropyl-propan-2-amine (80 μL, 0.459 mmol). The mixture was stirred at 20° C. for 2 hours. The residue was purified by preparative HPLC (Instrument: Gilson GX-281 Liquid Handler, Gilson 322 Pump, Gilson 156 UV Detector; Column: Phenomenex Gemini-NX 75×40 mm×3 m; Mobile phase A: H₂O with 10 mm NH₄HCO₃ (v %); Mobile phase B: MeCN; Gradient: B from 43% to 73% in 7.8 min, hold 100% B for 1 min; Flow Rate: 25 mL/min; Column Temperature: 30° C.; Wavelength: 220 nm, 254 nm) to afford N-imidazo[1,2-a]pyridin-7-yl-2-oxo-2-[(2R,5S)-5-methyl-2-[2-[rel-(3S,4S)-1,3-dimethyl-4-piperidyl]-1,3-benzothiazol-5-yl]-1-piperidyl]acetamide (28 mg, 36.3% yield) as white solid.

Compound 58: ¹H NMR (400 MHz, methanol-d₄) δ ppm 8.24-8.51 (m, 1H), 7.86-8.19 (m, 3H), 7.68-7.83 (m, 1H), 7.40-7.61 (m, 2H), 6.91-7.38 (m, 1H), 5.43-5.94 (m, 1H), 3.72-4.09 (m, 1H), 3.39-3.55 (m, 1H), 2.80-3.21 (m, 2H), 1.79-2.69 (m, 12H), 1.20-1.60 (m, 2H), 1.15 (d, J=6.8 Hz, ³H), 0.82-0.98 (m, ³H); LCMS(ESI) [M+H]⁺ m/z: calcd 531.2, found 531.3; HPLC: 100%@254 nm; SFC: 100% ee.

### Example 71. Synthesis of N-imidazo[1,2-a]pyridin-7-yl-2-oxo-2-[(2R,5S)-5-methyl-2-[2-[rel-(3R,4S)-1,3-dimethyl-4-piperidyl]-1,3-benzothiazol-5-yl]-1-piperidyl]acetamide (Compound 48)

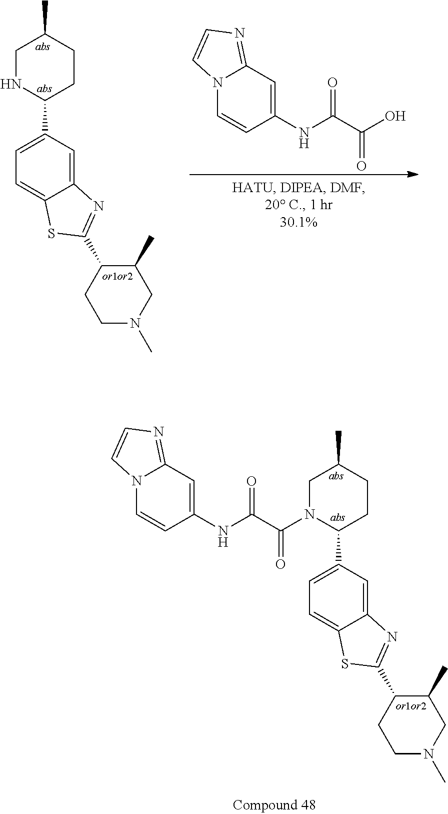 [SMILES: C[C@H]1CC[C@H](c2ccc3sc([C@H]4CCN(C)C[C@@H]4C)nc3c2)NC1 ; O=C(O)C(=O)Nc1ccn2ccnc2c1 ; C[C@H]1CC[C@H](c2ccc3sc([C@H]4CCN(C)C[C@@H]4C)nc3c2)N(C(=O)C(=O)Nc2ccn3ccnc3c2)C1]

### Step 1: Synthesis of N-imidazo[1,2-a]pyridin-7-yl-2-oxo-2-[(2R,5S)-5-methyl-2-[2-[rel-(3R,4S)-1,3-dimethyl-4-piperidyl]-1,3-benzothiazol-5-yl]-1-piperidyl]acetamide (Compound 48)

To a solution of 2-[rel-(3R,4S)-1,3-dimethyl-4-piperidyl]-5-[(2R,5S)-5-methyl-2-piperidyl]-1,3-benzothiazole (50 mg, 0.132 mmol, HCl), 2-(imidazo[1,2-a]pyridin-7-ylamino)-2-oxo-acetic acid (30 mg, 0.146 mmol) in DMF (5.00 mL) was added HATU (75 mg, 198 mmol), DIPEA (100 μL, 0.574 mmol). The mixture was stirred at 20° C. for 1 hour. The resulting mixture was concentrated under reduced pressure. The residue was purified by preparative HPLC (Instrument: Gilson GX-281 Liquid Handler, Gilson 322 Pump, Gilson 156 UV Detector; Column: Phenomenex C18 80*40 mm*3 μm; Mobile phase A: H₂O with 0.05% NH₃—H₂O (v %); Mobile phase B: MeCN; Gradient: B from 40% to 70% in 7.8 min, hold 100% B for 1 min; Flow Rate: 25 mL/min; Column Temperature: 30° C.; Wavelength: 220 nm, 254 nm) to afford N-imidazo[1,2-a]pyridin-7-yl-2-oxo-2-[(2R,5S)-5-methyl-2-[2-[rel-(3R,4S)-1,3-dimethyl-4-piperidyl]-1,3-benzothiazol-5-yl]-1-piperidyl]acetamide (21 mg, 30.1% yield, single unknown enantiomer with trans relative chemistry, white solid)

Compound 48: ¹H NMR (400 MHZ, methanol-d₄) δ ppm 8.27-8.49 (m, 1H), 8.14 (br s, 1H), 7.89-8.07 (m, 2H), 7.68-7.86 (m, 1H), 7.44-7.60 (m, 2H), 7.20 (br d, J=7.0 Hz, 1H), 7.03 (br s, 1H), 5.86 (br s, 1H), 5.48 (br s, 1H), 4.06 (br d, J=12.8 Hz, 1H), 3.78 (br d, J=13.8 Hz, 1H), 3.49 (br d, J=13.6 Hz, 1H), 3.19-3.29 (m, 1H), 3.03 (br s, 2H), 2.67-2.89 (m, 1H), 2.36 (br s, 5H), 1.77-2.24 (m, 7H), 1.51 (br s, 1H), 1.27-1.38 (m, 1H), 1.17 (d, J=7.0 Hz, ³H), 0.77-0.96 (m, ³H). LCMS(ESI) [M+H]⁺ m/z: calcd 531.3, found 531.2; HPLC: 96.13%@254 nm; SFC: 94.2% ee.

### Example 72. Synthesis of -[(2R,5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (Compound 43)

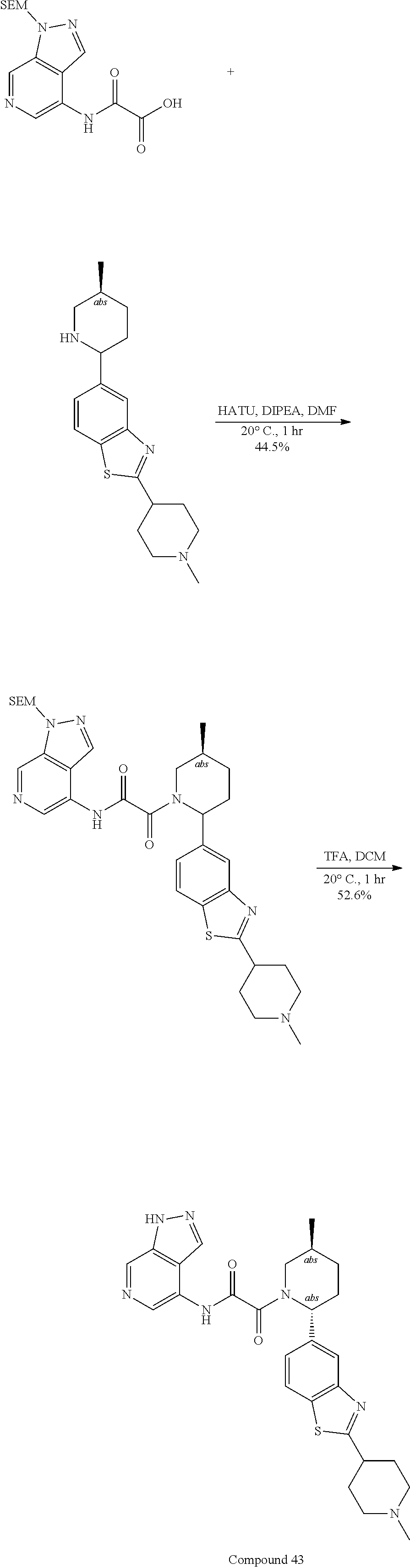 [SMILES: C[Si](C)(C)CCOCn1ncc2c(NC(=O)C(=O)O)cncc21 ; C[C@H]1CCC(c2ccc3sc(C4CCN(C)CC4)nc3c2)NC1 ; C[C@H]1CCC(c2ccc3sc(C4CCN(C)CC4)nc3c2)N(C(=O)C(=O)Nc2cncc3c2cnn3COCC[Si](C)(C)C)C1 ; C[C@H]1CC[C@H](c2ccc3sc(C4CCN(C)CC4)nc3c2)N(C(=O)C(=O)Nc2cncc3[nH]ncc23)C1]

### Step 1: The Synthesis of 2-(4-fluorophenyl)-3-methyl-pyrazine

To a mixture of 2-oxo-2-[[1-(2-trimethylsilylethoxymethyl)pyrazolo[3,4-c]pyridin-4-yl]amino]acetic acid 70 mg, 0.21 mmol) and 2-(1-methyl-4-piperidyl)-5-[(5S)-5-methyl-2-piperidyl]-1,3-benzothiazole (Intermediate 32)(82 mg, 0.25 mmol) in DCM (3 mL) were added HATU (102 mg, 0.27 mmol) and N-ethyl-N-isopropyl-propan-2-amine (148 mg, 1.15 mmol) and the mixture was stirred at 20° C. for 1 hour. The mixture was concentrated under reduce pressure and the residue was purified by flash chromatography (ISCO; 4 g AgelaFlash Silica Flash Column, DCM/MeOH with MeOH from 0˜10%, flow rate=30 mL/min, 254 nm) to afford 2-[(5S)-5-methyl-2-[2-(1-methyl-4-piperidyl)-1,3-benzothiazol-5-yl]-1-piperidyl]-2-oxo-N-[1-(2-trimethylsilylethoxymethyl)pyrazolo[3,4-c]pyridin-4-yl]acetamide (60 mg, 44.5% yield) as a light-yellow solid. LCMS(ESI) [M+H]⁺ m/z: calcd 648.3, found 324.6 (M/2+H cleaved mass).

### Step 2: The Synthesis of -[(2R,5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (Compound 43)

A mixture of 2-[(5S)-5-methyl-2-[2-(1-methyl-4-piperidyl)-1,3-benzothiazol-5-yl]-1-piperidyl]-2-oxo-N-[1-(2-trimethylsilylethoxymethyl)pyrazolo[3,4-c]pyridin-4-yl]acetamide (55 mg, 84.9 μmol) in TFA (2.96 g, 26.0 mmol) and DCM (2 mL) was stirred at 20° C. for 1 hour. The mixture was filtered and the filtrate was concentrated under reduced pressure. The residue was purified by preparative HPLC purification (Instrument: Gilson GX-281 Liquid Handler, Gilson 322 Pump, Gilson 156 UV Detector; Column: Durashell 150×25 mm×5 μm; Mobile phase A: water (10 mM NH₄HCO₃); Mobile phase B: MeCN; Gradient: B from 23% to 53% in 9.5 min, hold 100% B for 2.0 min; Flow Rate: 25 mL/min; Column Temperature: 30° C.; Wavelength: 220 nm, 254 nm) to 2-[(2R,5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (23.1 mg, 52.6% yield) as a white solid. ¹H NMR (400 MHZ, methanol-d₄) δ ppm 8.72-8.91 (m, 1H), 8.46-8.70 (m, 1H), 8.12-8.43 (m, 1H), 7.87-8.06 (m, 2H), 7.44-7.58 (m, 1H), 5.45-5.98 (m, 1H), 3.74-4.15 (m, 1H), 3.41-3.59 (m, 1H), 3.05-3.27 (m, 3H), 2.44 (s, 9H), 1.90-2.14 (m, 4H), 1.45-1.57 (m, 1H), 1.16-1.28 (m, ³H);

LCMS(ESI) [M+H]⁺ m/z: calcd 518.2, found 518.1; HPLC: 100%@220 nm, 100%@254 nm; 100% ee.

### Example 73. Synthesis of 2-((2R,5S)-5-methyl-2-(2-(1-methylpiperidin-4-yl)benzo[d]thiazol-5-yl)piperidin-1-yl)-2-oxo-N-(1H-pyrazolo[3,4-c]pyridin-4-yl)acetamide (Compound 43)

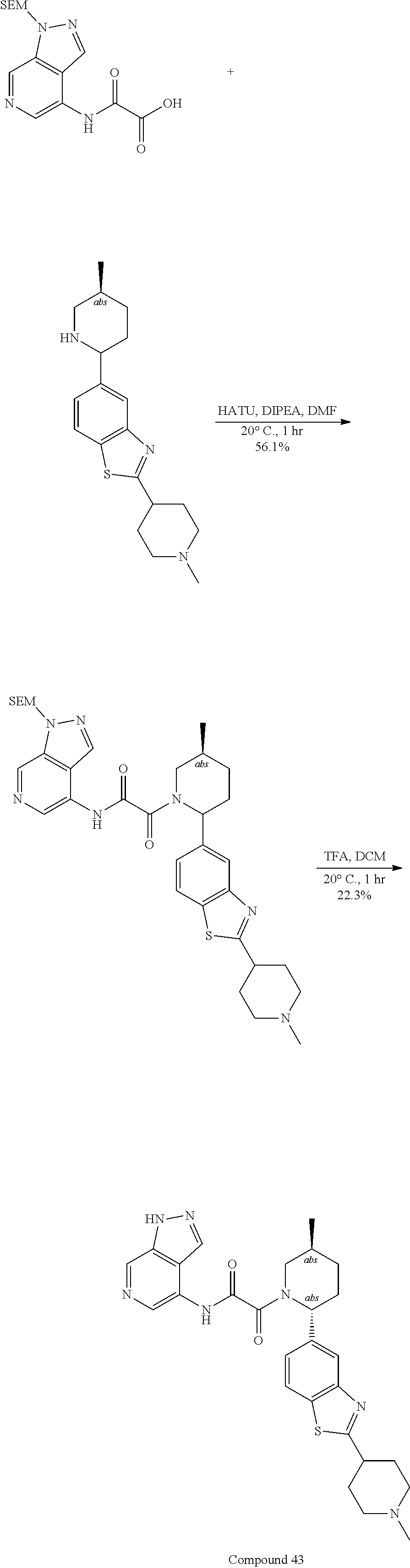 [SMILES: C[Si](C)(C)CCOCn1ncc2c(NC(=O)C(=O)O)cncc21 ; C[C@H]1CCC(c2ccc3sc(C4CCN(C)CC4)nc3c2)NC1 ; C[C@H]1CCC(c2ccc3sc(C4CCN(C)CC4)nc3c2)N(C(=O)C(=O)Nc2cncc3c2cnn3COCC[Si](C)(C)C)C1 ; C[C@H]1CC[C@H](c2ccc3sc(C4CCN(C)CC4)nc3c2)N(C(=O)C(=O)Nc2cncc3[nH]ncc23)C1]

### Step 1: The Synthesis of 2-(4-fluorophenyl)-3-methyl-pyrazine

To a mixture of 2-oxo-2-[[1-(2-trimethylsilylethoxymethyl)pyrazolo[3,4-c]pyridin-4-yl]amino]acetic acid (250 mg, 0.74 mmol) and 2-(1-methyl-4-piperidyl)-5-[(5S)-5-methyl-2-piperidyl]-1,3-benzothiazole (Intermediate 32)(300 mg, 0.91 mmol) in DMF (3 mL) and DCM (5 mL) were added HATU (370 mg, 0.97 mmol) and N-ethyl-N-isopropyl-propan-2-amine (445 mg, 3.44 mmol) and the mixture was stirred at 20° C. for 12 hours. The reaction mixture was diluted with EtOAc (50 mL) and washed with brine (100 mL*3), dried over Na₂SO₄, concentrated under reduce pressure. The residue was purified by flash chromatography (ISCO; 40 g AgelaFlash Silica Flash Column, petroleum DCM/MeOH with EtOAc from 0˜10%, flow rate=30 mL/min, 254 nm) to afford 2-[(5S)-5-methyl-2-[2-(1-methyl-4-piperidyl)-1,3-benzothiazol-5-yl]-1-piperidyl]-2-oxo-N-[1-(2-trimethylsilylethoxymethyl)pyrazolo[3,4-c]pyridin-4-yl]acetamide (270 mg, 56.1% yield) as a brown oil. LCMS(ESI) [M+H]⁺ m/z: calcd 648.1, found 648.2.

### Step 2: The Synthesis of 2-((2R,5S)-5-methyl-2-(2-(1-methylpiperidin-4-yl)benzo[d]thiazol-5-yl)piperidin-1-yl)-2-oxo-N-(1H-pyrazolo[3,4-c]pyridin-4-yl)acetamide (Compound 43)

A mixture of 2-[(5S)-5-methyl-2-[2-(1-methyl-4-piperidyl)-1,3-benzothiazol-5-yl]-1-piperidyl]-2-oxo-N-[1-(2-trimethylsilylethoxymethyl)pyrazolo[3,4-c]pyridin-4-yl]acetamide (250 mg, 0.39 mmol) in TFA (5 mL, 64.9 mmol) and DCM (5 mL) was stirred at 20° C. for 1 hour. The mixture was concentrated under reduce pressure and the residue was sent to preparative HPLC (Instrument: Gilson GX-281 Liquid Handler, Gilson 322 Pump, Gilson 156 UV Detector; Column: Durashell 150×25 mm×5 μm; Mobile phase A: H₂O with 0.05% NH₃—H₂O (v %); Mobile phase B: MeCN; Gradient: B from 24% to 54% in 9.5 min, hold 100% B for 2.0 min; Flow Rate: 25 mL/min; Column Temperature: 30° C.; Wavelength: 220 nm, 254 nm) to afford 2-((2R,5S)-5-methyl-2-(2-(1-methylpiperidin-4-yl)benzo[d]thiazol-5-yl)piperidin-1-yl)-2-oxo-N-(1H-pyrazolo[3,4-c]pyridin-4-yl)acetamide (44.5 mg, 22.3% yield) as a white solid. ¹H NMR (400 MHZ, methanol-d₄) δ ppm 8.87 (br s, 1H), 8.75 (br d, J=2.3 Hz, 1H), 8.66 (s, 1H), 8.49-8.53 (m, 1H), 8.67 (d, J=4.8 Hz, 1H), 8.12-8.44 (m, 1H), 8.11-8.16 (m, 1H), 7.91-8.06 (m, 2H), 7.48-7.54 (m, 1H), 5.45-5.94 (m, 1H), 5.45-5.93 (m, 1H), 5.40-5.53 (m, 1H), 4.80-4.84 (m, 1H), 3.79-3.86 (m, 1H), 3.74-4.11 (m, 1H), 3.38-3.57 (m, 1H), 3.05-3.25 (m, 3H), 2.35-2.46 (m, 6H), 2.13-2.34 (m, 3H), 1.94-2.10 (m, 4H), 1.47-1.55 (m, 1H), 1.30-1.38 (m, 1H), 1.21 (br d, J=5.5 Hz, ³H); LCMS(ESI) [M+H]⁺ m/z: calcd 518.2, found 518.3; HPLC: 100%@220 nm, 100%@254 nm; 100% ee.

### Example 74. Synthesis of 2-[(2R,5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (Compound 43)

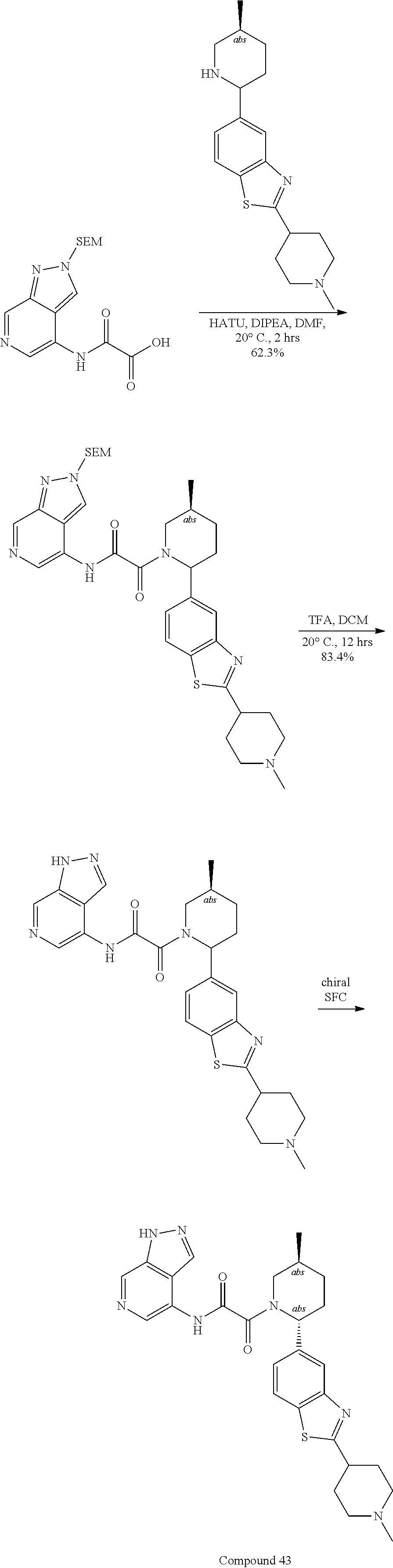 [SMILES: C[Si](C)(C)CCOCn1cc2c(NC(=O)C(=O)O)cncc2n1 ; C[C@H]1CCC(c2ccc3sc(C4CCN(C)CC4)nc3c2)NC1 ; C[C@H]1CCC(c2ccc3sc(C4CCN(C)CC4)nc3c2)N(C(=O)C(=O)Nc2cncc3nn(COCC[Si](C)(C)C)cc23)C1 ; C[C@H]1CCC(c2ccc3sc(C4CCN(C)CC4)nc3c2)N(C(=O)C(=O)Nc2cncc3[nH]ncc23)C1 ; C[C@H]1CC[C@H](c2ccc3sc(C4CCN(C)CC4)nc3c2)N(C(=O)C(=O)Nc2cncc3[nH]ncc23)C1]

### Step 1: Synthesis of 2-[(5S)-5-methyl-2-[2-(1-methyl-4-piperidyl)-1,3-benzothiazol-5-yl]-1-piperidyl]-2-oxo-N-[2-(2-trimethylsilylethoxymethyl)pyrazolo[3,4-c]pyridin-4-yl]acetamide

To a mixture of 2-oxo-2-[[2-(2-trimethylsilylethoxymethyl)pyrazolo[3,4-c]pyridin-4-yl]amino]acetic acid (Intermediate 42)(250 mg, 0.743 mmol) and 2-(1-methyl-4-piperidyl)-5-[(5S)-5-methyl-2-piperidyl]-1,3-benzothiazole (Intermediate 32)(290 mg, 0.880 mmol) in DMF (8 mL) were added HATU (350 mg, 0.921 mmol) and DIPEA (0.65 mL, 3.73 mmol). The resulting mixture was stirred at 20° C. for 2 hours. The resulting mixture was quenched by addition of water (50 mL) and extracted with EtOAc (100 mL*3). The combined organic layer was washed with saturated NH₄Cl aqueous solution (50 mL), brine (100 mL), dried over anhydrous Na₂SO₄, filtered and concentrated under reduced pressure. The residue was purified by flash chromatography (ISCO®; 12 g AgelaFlash® Silica Flash Column,

DCM/MeOH with MeOH from 0˜10%, flow rate=30 mL/min, 254 nm) to afford 2-[(5S)-5-methyl-2-[2-(1-methyl-4-piperidyl)-1,3-benzothiazol-5-yl]-1-piperidyl]-2-oxo-N-[2-(2-trimethylsilylethoxymethyl)pyrazolo[3,4-c]pyridin-4-yl]acetamide (300 mg, 62.3% yield) as yellow solid.

### Step 2: Synthesis of 2-[(5S)-5-methyl-2-[2-(1-methyl-4-piperidyl)-1,3-benzothiazol-5-yl]-1-piperidyl]-2-oxo-N-(1H-pyrazolo[4,3-c]pyridin-7-yl)acetamide

A mixture of 2-[(5S)-5-methyl-2-[2-(1-methyl-4-piperidyl)-1,3-benzothiazol-5-yl]-1-piperidyl]-2-oxo-N-[2-(2-trimethylsilylethoxymethyl)pyrazolo[4,3-c]pyridin-7-yl]acetamide (300 mg, 0.463 mmol), 2-[(5S)-5-methyl-2-[2-(1-methyl-4-piperidyl)-1,3-benzothiazol-5-yl]-1-piperidyl]-2-oxo-N-[1-(2-trimethylsilylethoxymethyl)pyrazolo[4,3-c]pyridin-7-yl]acetamide (250 mg, 0.386 mmol), DCM (5 mL) and TFA (5 mL, 64.9 mmol) was stirred at 20° C. for 12 hours. The mixture was concentrated under reduced pressure. The residue was purified by preparative HPLC (Instrument: Gilson GX-281 Liquid Handler, Gilson 322 Pump, Gilson 156 UV Detector; Column: Phenomenex Gemini-NX 80*40 mm*3 μm; Mobile phase A: H₂O with 10 mmol NH₄HCO₃ (v %); Mobile phase B: MeCN; Gradient: B from 23% to 53% in 7.8 min, hold 100% B for 1 min; Flow Rate: 25 mL/min; Column Temperature: 30° C.; Wavelength: 220 nm, 254 nm) to afford 2-[(5S)-5-methyl-2-[2-(1-methyl-4-piperidyl)-1,3-benzothiazol-5-yl]-1-piperidyl]-2-oxo-N-(1H-pyrazolo[4,3-c]pyridin-7-yl)acetamide (200 mg, 83.4% yield) as white solid. LCMS(ESI) [M+H]⁺ m/z: calcd 518.2, found 518.3; HPLC: 98.55%@220 nm, 98.77%@254 nm.

### Step 3: Synthesis of 2-[(2R,5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (Compound 43)

2-[(5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (200 mg, 0.386 mmol) was purified by SFC (Instrument: Berger, Multigr AM-II; Column: Daicel Chiralpak IG (250 mm*30 mm*10 μm); Mobile phase: supercritical CO₂/IPA (0.1% NH₃—H₂O, v %)=60/40; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm). The fraction was concentrated under reduced pressure and then lyophilized for overnight to give Compound 43.

Compound 43:2-[(2R,5S)-5-methyl-2-[2-(1-methyl-4-piperidyl)-1,3-benzothiazol-5-yl]-1-piperidyl]-2-oxo-N-(1H-pyrazolo[3,4-c]pyridin-4-yl)acetamide (166.5 mg, single known enantiomer with trans relative chemistry, Peak 2, Retention time: 4.227 min, white solid). ¹H NMR (400 MHZ, methanol-d₄) δ ppm 8.69-8.92 (m, 1H), 8.45-8.67 (m, 1H), 8.09-8.42 (m, 1H), 7.82-8.04 (m, 1H), 7.47 (s, 1H), 5.37-5.95 (m, 1H), 3.79 (d, J=13.6 Hz, 1H), 3.50 (d, J=14.3 Hz, 1H), 3.00 (d, J=9.3 Hz, ³H), 2.33 (s, 5H), 2.11-2.29 (m, 4H), 1.79-2.09 (m, 4H), 1.49 (d, J=11.0 Hz, 1H), 1.18 (s, ³H); LCMS(ESI) [M+H]⁺ m/z: calcd 518.2, found 518.3; HPLC: 100%@220 nm, 100%@254 nm; 100% ee.

### Example 75. Synthesis of 2-[(2R,5S)-5-methyl-2-[2-[(3R)-1-methylpyrrolidin-3-yl]-1,3-benzothiazol-5-yl]-1-piperidyl]-2-oxo-N-(1H-pyrazolo[4,3-c]pyridin-7-yl)acetamide (Compound 30) and 2-[(2R,5S)-5-methyl-2-[2-[(3S)-1-methylpyrrolidin-3-yl]-1,3-benzothiazol-5-yl]-1-piperidyl]-2-oxo-N-(1H-pyrazolo[4,3-c]pyridin-7-yl)acetamide (Compound 2)

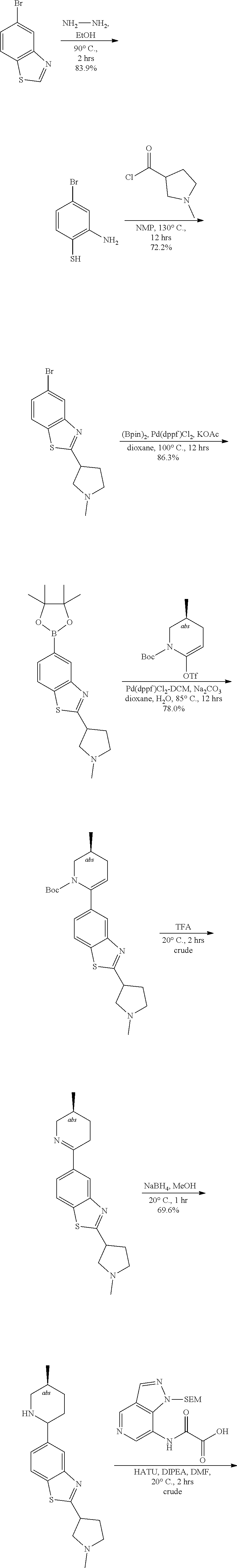

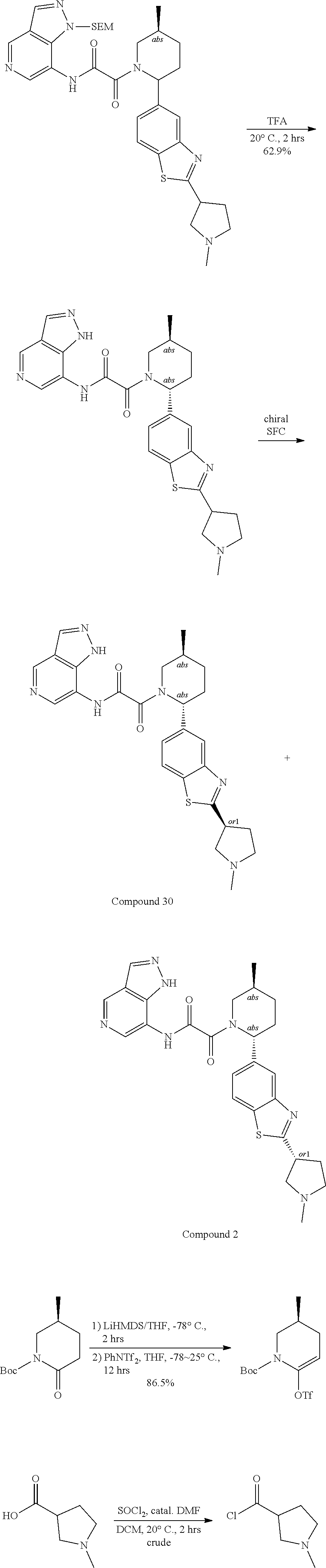

### Step 1: Synthesis of 2-amino-4-bromo-benzenethiol

A mixture of 5-bromo-1,3-benzothiazole (4 g, 18.9 mmol), hydrazine; hydrate (10 mL, 0.174 mol, 85% purity) was stirred at 90° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give 2-amino-4-bromo-benzenethiol (3.2 g, 83.9% yield) as yellow solid. ¹H NMR (400 MHz, methanol-d₄) δ ppm 7.13-7.14 (m, 1H), 7.14 (d, J=8.3 Hz, 1H), 6.92-6.96 (m, 1H), 6.60-6.72 (m, 1H).

### Step 2: Synthesis of 5-bromo-2-(1-methylpyrrolidin-3-yl)-1,3-benzothiazole

A mixture of 1-methylpyrrolidine-3-carbonyl chloride (1.1 g, 7.45 mmol), 2-amino-4-bromo-benzenethiol (1.6 g, 7.84 mmol) and NMP (10 mL) was stirred at 130° C. for 12 hours. The mixture was purified by flash chromatography (Column: SepaFlash®Sphercial C18, 25 g, 40-60 μm, 120 Å; MeCN/water (0.5% NH₃—H₂O) with MeCN from 0-55%, 25 mL/min, 220 nm) to give 5-bromo-2-(1-methylpyrrolidin-3-yl)-1,3-benzothiazole (1.6 g, 72.2% yield) as yellow oil. ¹H NMR (400 MHZ, meyanol-d₄) δ ppm 7.97 (d, J=1.8 Hz, 1H), 7.77 (d, J=8.5 Hz, 1H), 7.43 (dd, J=8.6, 1.8 Hz, 1H), 3.79-3.89 (m, 1H), 3.04 (dd, J=9.8, 7.9 Hz, 1H), 2.84 (dd, J=9.8, 6.6 Hz, 1H), 2.66-2.75 (m, 2H), 2.32-2.47 (m, 4H), 2.11-2.19 (m, 1H).

### Step 3: Synthesis of 2-(1-methylpyrrolidin-3-yl)-5-(4,4,5,5-tetramethyl-1,3,2-dioxaborolan-2-yl)-1,3-benzothiazole

A mixture of 5-bromo-2-(1-methylpyrrolidin-3-yl)-1,3-benzothiazole (1.5 g, 5.05 mmol), KOAc (2.86 g, 10.2 mmol), 4,4,5,5-tetramethyl-2-(4,4,5,5-tetramethyl-1,3,2-dioxaborolan-2-yl)-1,3,2-dioxaborolane (1.95 g, 7.70 mmol), cyclopentyl(diphenyl)phosphane; dichloropalladium; iron (400 mg, 0.547 mmol) and dioxane (20 mL) was stirred at 100° C. for 12 hours. The resulting mixture was quenched by addition of water (50 mL) and extracted with EtOAc (100 mL*3). The combined organic layer was washed with saturated NH₄Cl aqueous solution (50 mL), brine (100 mL), dried over anhydrous Na₂SO₄, filtered and concentrated under reduced pressure. The residue was purified by flash chromatography (ISCO®; 80 g AgelaFlash® Silica Flash Column, EtOAc/MeOH with MeOH from 0˜10%, Flow Rate: 30 mL/min, 254 nm) to afford 2-(1-methylpyrrolidin-3-yl)-5-(4,4,5,5-tetramethyl-1,3,2-dioxaborolan-2-yl)-1,3-benzothiazole (1.5 g, 86.3% yield) as brown solid. ¹H NMR (400 MHZ, methanol-d₄) δ ppm 8.27-8.37 (m, 1H), 7.92-8.03 (m, 1H), 7.72-7.81 (m, 1H), 4.07-4.22 (m, 1H), 3.47-3.64 (m, 2H), 3.21-3.29 (m, 2H), 2.79-2.83 (m, 3H), 2.64 (br dd, J=8.7, 6.7 Hz, 1H), 2.36-2.44 (m, 1H), 1.40 (s, 7H), 1.22 (s, 5H). LCMS(ESI) [M+H]⁺ m/z: calcd 345.2, found 345.2.

### Step 4: Synthesis of tert-butyl (3S)-3-methyl-6-[2-(1-methylpyrrolidin-3-yl)-1,3-benzothiazol-5-yl]-3,4-dihydro-2H-pyridine-1-carboxylate

To a mixture of 2-(1-methylpyrrolidin-3-yl)-5-(4,4,5,5-tetramethyl-1,3,2-dioxaborolan-2-yl)-1,3-benzothiazole (1.6 g, 4.65 mmol), tert-butyl (3S)-3-methyl-6-(trifluoromethylsulfonyloxy)-3,4-dihydro-2H-pyridine-1-carboxylate (3 g, 8.69 mmol) in dioxane (20 mL) and H₂O (5 mL) were added Pd(dppf)Cl₂-DCM (480 mg, 0.588 mmol) and Na₂CO₃ (1.60 g, 15.1 mmol). The resulting mixture was sealed and degassed under vacuum and purged with N₂ for three times, and then stirred at 85° C. for 12 hours under N₂ atmosphere. The mixture was filtered and concentrated under reduced pressure. The residue was purified by flash chromatography (ISCO®; 24 g AgelaFlash® Silica Flash Column, EtOAc/MeOH with MeOH from 0˜20%, Flow Rate: 30 mL/min) to afford tert-butyl (3S)-3-methyl-6-[2-(1-methylpyrrolidin-3-yl)-1,3-benzothiazol-5-yl]-3,4-dihydro-2H-pyridine-1-carboxylate (1.5 g, 78.0% yield) as yellow oil. ¹H NMR (400 MHZ, methanol-d₄) δ ppm 7.89 (d, J=8.3 Hz, 1H), 7.83 (d, J=1.3 Hz, 1H), 7.38 (dd, J=8.4, 1.6 Hz, 1H), 5.48 (t, J=3.6 Hz, 1H), 4.02 (dd, J=12.2, 3.1 Hz, 1H), 3.10-3.17 (m, 2H), 2.93-3.03 (m, 2H), 2.58 (s, 2H), 2.44-2.55 (m, 1H), 2.25-2.36 (m, 2H), 1.80-2.18 (m, 4H), 1.01-1.08 (m, 12H).

### Step 5: Synthesis of 2-(1-methylpyrrolidin-3-yl)-5-[(3S)-3-methyl-2,3,4,5-tetrahydropyridin-6-yl]-1,3-benzothiazole

A mixture of tert-butyl (3S)-3-methyl-6-[2-(1-methylpyrrolidin-3-yl)-1,3-benzothiazol-5-yl]-3,4-dihydro-2H-pyridine-1-carboxylate (1.50 g, 3.63 mmol) and TFA (10 mL, 0.130 mol) was stirred at 20° C. for 2 hours. The mixture was concentrated under reduced pressure to give 2-(1-methylpyrrolidin-3-yl)-5-[(3S)-3-methyl-2,3,4,5-tetrahydropyridin-6-yl]-1,3-benzothiazole (1.2 g, crude) as yellow oil.

### Step 6: Synthesis of 5-[(5S)-5-methyl-2-piperidyl]-2-(1-methylpyrrolidin-3-yl)-1,3-benzothiazole

To a mixture of 2-(1-methylpyrrolidin-3-yl)-5-[(3S)-3-methyl-2,3,4,5-tetrahydropyridin-6-yl]-1,3-benzothiazole (1 g, 3.19 mmol) and MeOH (20 mL) was added NaBH₄ (300 mg, 7.93 mmol). The mixture was stirred at 20° C. for 1 hour. The mixture was purified by (Column: SepaFlash®Sphercial C18, 25 g, 40-60 μm, 120 Å; MeCN/water (0.5% NH₃—H₂O) with MeCN from 0˜50%, 25 mL/min, 220 nm) to give 5-[(5S)-5-methyl-2-piperidyl]-2-(1-methylpyrrolidin-3-yl)-1,3-benzothiazole (700 mg, 69.6% yield) as yellow oil.

### Step 7: Synthesis of 2-[(5S)-5-methyl-2-[2-(1-methylpyrrolidin-3-yl)-1,3-benzothiazol-5-yl]-1-piperidyl]-2-oxo-N-[1-(2-trimethylsilylethoxymethyl)pyrazolo[4,3-c]pyridin-7-yl]acetamide

A mixture of 2-oxo-2-[[1-(2-trimethylsilylethoxymethyl)pyrazolo[4,3-c]pyridin-7-yl]amino]acetic acid (Intermediate 37)(260 mg, 0.773 mmol), 5-[(5S)-5-methyl-2-piperidyl]-2-(1-methylpyrrolidin-3-yl)-1,3-benzothiazole (220 mg, 0.697 mmol), HATU (350 mg, 0.920 mmol), DIPEA (2 mL, 11.5 mmol) and DMF (4 mL) was stirred at 20° C. for 2 hours. The mixture was concentrated under reduced pressure. The residue was purified by flash chromatography (ISCO®; 24 g AgelaFlash® Silica Flash Column, DCM/MeOH with MeOH from 0˜20%, Flow Rate: 30 mL/min, 254 nm) to afford 2-[(5S)-5-methyl-2-[2-(1-methylpyrrolidin-3-yl)-1,3-benzothiazol-5-yl]-1-piperidyl]-2-oxo-N-[1-(2-trimethylsilylethoxymethyl)pyrazolo[4,3-c]pyridin-7-yl]acetamide (500 mg, crude) as yellow oil.

### Step 8: Synthesis of 2-[(5S)-5-methyl-2-[2-(1-methylpyrrolidin-3-yl)-1,3-benzothiazol-5-yl]-1-piperidyl]-2-oxo-N-(1H-pyrazolo[4,3-c]pyridin-7-yl)acetamide

A mixture of 2-[(5S)-5-methyl-2-[2-(1-methylpyrrolidin-3-yl)-1,3-benzothiazol-5-yl]-1-piperidyl]-2-oxo-N-[1-(2-trimethylsilylethoxymethyl)pyrazolo[4,3-c]pyridin-7-yl]acetamide (400 mg, 0.631 mmol) and TFA (3 mL, 38.9 mmol) was stirred at 20° C. for 2 hours. The residue was purified by preparative HPLC (Instrument: Gilson GX-281 Liquid Handler, Gilson 322 Pump, Gilson 156 UV Detector; Column: Waters Xbridge 150×25 mm×5 um; Mobile phase A: H₂O with 0.05% NH₃—H₂O (v %); Mobile phase B: MeCN; Gradient: B from 23% to 53% in 9.5 min, hold 100% B for 2.5 min; Flow Rate: 25 mL/min; Column Temperature: 30° C.; Wavelength: 220 nm, 254 nm) to afford 2-[(5S)-5-methyl-2-[2-(1-methylpyrrolidin-3-yl)-1,3-benzothiazol-5-yl]-1-piperidyl]-2-oxo-N-(1H-pyrazolo[4,3-c]pyridin-7-yl)acetamide (200 mg, 63.0% yield) as white solid. LCMS(ESI) [M+H]⁺ m/z: calcd 504.2; found 504.2.

### Step 9: Synthesis of 2-[(2R,5S)-5-methyl-2-[2-[rel-(3R)-1-methylpyrrolidin-3-yl]-1,3-benzothiazol-5-yl]-1-piperidyl]-2-oxo-N-(1H-pyrazolo[4,3-c]pyridin-7-yl)acetamide (Compound 30) and 2-[(2R,5S)-5-methyl-2-[2-[rel-(3S)-1-methylpyrrolidin-3-yl]-1,3-benzothiazol-5-yl]-1-piperidyl]-2-oxo-N-(1H-pyrazolo[4,3-c]pyridin-7-yl)acetamide (Compound 2)

2-[(2R,5S)-5-methyl-2-[2-(1-methylpyrrolidin-3-yl)-1,3-benzothiazol-5-yl]-1-piperidyl]-2-oxo-N-(1H-pyrazolo[4,3-c]pyridin-7-yl)acetamide (40 mg, 0.0794 mmol) was purified by SFC (Instrument: Thar 800Q; Daicel chiralpak IG (250 mm*30 mm*10 μm); Mobile phase: supercritical CO₂/EtOH (0.1% NH₃—H₂O, v %)=60/40; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to give Compound 30 and Compound 2.

Compound 30:2-[(2R,5S)-5-methyl-2-[2-[(3R)-1-methylpyrrolidin-3-yl]-1,3-benzothiazol-5-yl]-1-piperidyl]-2-oxo-N-(1H-pyrazolo[4,3-c]pyridin-7-yl)acetamide (20 mg, single unknown enantiomer, peak 1, retention time=5.086 min, white solid). ¹H NMR (400 MHZ, methanol-d₄) δ ppm 8.71-8.96 (m, 1H), 8.10-8.39 (m, 2H), 7.68-7.94 (m, 2H), 7.37 (d, J=6.8 Hz, 1H), 5.43-5.95 (m, 1H), 3.76-4.09 (m, 2H), 3.35-3.48 (m, 1H), 3.14 (d, J=9.0 Hz, 1H), 2.84-2.97 (m, 1H), 2.69-2.83 (m, 2H), 2.41 (s, 4H), 2.09-2.31 (m, 3H), 1.78-1.98 (m, 2H), 1.39 (d, J=12.5 Hz, 1H), 1.08 (d, J=6.8 Hz, ³H). LCMS(ESI) [M+H]⁺ m/z: calcd 504.2, found 504.2; HPLC: 97.28%@220 nm, 100%@254 nm, 97.0% ee.

Compound 2:2-[(2R,5S)-5-methyl-2-[2-[(3S)-1-methylpyrrolidin-3-yl]-1,3-benzothiazol-5-yl]-1-piperidyl]-2-oxo-N-(1H-pyrazolo[4,3-c]pyridin-7-yl)acetamide (20 mg, single unknown enantiomer, peak 2, retention time=5.928 min, white solid). ¹H NMR (400 MHZ, methanol-d₄) δ ppm 8.76-9.15 (m, 1H), 8.17-8.53 (m, 2H), 7.86-8.10 (m, 2H), 7.50 (s, 1H), 5.49-6.00 (m, 1H), 3.91-4.18 (m, 2H), 3.52 (d, J=13.6 Hz, 1H), 3.26 (s, 1H), 2.97-3.12 (m, 1H), 2.85-2.96 (m, 2H), 2.54 (s, 4H), 2.20-2.44 (m, 3H), 1.87-2.11 (m, 2H), 1.51 (d, J=11.5 Hz, 1H), 1.20 (d, J=6.8 Hz, ³H). LCMS(ESI) [M+H]⁺ m/z: calcd 504.2, found 504.2; HPLC: 97.82%@220 nm, 100%@254 nm, 95.9% ee.

### Step 10: Synthesis of tert-butyl (3S)-3-methyl-6-(trifluoromethylsulfonyloxy)-3,4-dihydro-2H-pyridine-1-carboxylate

To a mixture of tert-butyl (5S)-5-methyl-2-oxo-piperidine-1-carboxylate (5 g, 23.4 mmol) in THF (90 mL) was sealed and degassed under vacuum and purged with N₂ for three times, and then 1M LiHMDS/THF (44.0 mL, 44.0 mmol) was added at −78° C. dropwise. The mixture was stirred 2 hours at −78° C. Then a solution of PhNTf₂ (13 g, 36.3 mmol) in THF (45 mL) was added. The solution was stirred at 25° C. for 12 hours. The reaction was quenched by addition of saturated NH₄Cl aqueous solution (100 mL), and then extracted with EtOAc (100 mL*3). The combined organic layers were dried over anhydrous Na₂SO₄, filtered and concentrated under reduced pressure. The residue was purified by flash chromatography (ISCO®; 80 g AgelaFlash® Silica Flash Column, petroleum ether/EtOAc with EtOAc from 0˜0%, flow rate=60 mL/min) to afford tert-butyl (3S)-3-methyl-6-(trifluoromethylsulfonyloxy)-3,4-dihydro-2H-pyridine-1-carboxylate (7 g, 86.5% yield) as light yellow oil. LCMS(ESI) [M+H]⁺ m/z: calcd 290.1; found 290.0 (t-Bu claved mass).

### Step 11: Synthesis of 1-methylpyrrolidine-3-carbonyl chloride

A mixture of 1-methylpyrrolidine-3-carboxylic acid (1 g, 7.74 mmol), SOCl₂ (9 g, 75.6 mmol), DCM (10 mL) and DMF (50 mg, 0.684 mmol) was stirred at 30° C. for 2 hours. The mixture was concentrated under reduced pressure to give 1-methylpyrrolidine-3-carbonyl chloride (1.2 g, crude) as yellow oil.

### Example 76. Synthesis of 2-oxo-N-(1H-pyrazolo[4,3-c]pyridin-7-yl)-2-[(2R,5S)-5-methyl-2-[2-[rel-(3R,4S)-1,3-dimethyl-4-piperidyl]-1,3-benzothiazol-5-yl]-1-piperidyl]acetamide (Compound 14) and 2-oxo-N-(1H-pyrazolo[4,3-c]pyridin-7-yl)-2-[(2R,5S)-5-methyl-2-[2-[rel-(3R,4R)-1,3-dimethyl-4-piperidyl]-1,3-benzothiazol-5-yl]-1-piperidyl]acetamide (Compound 12)

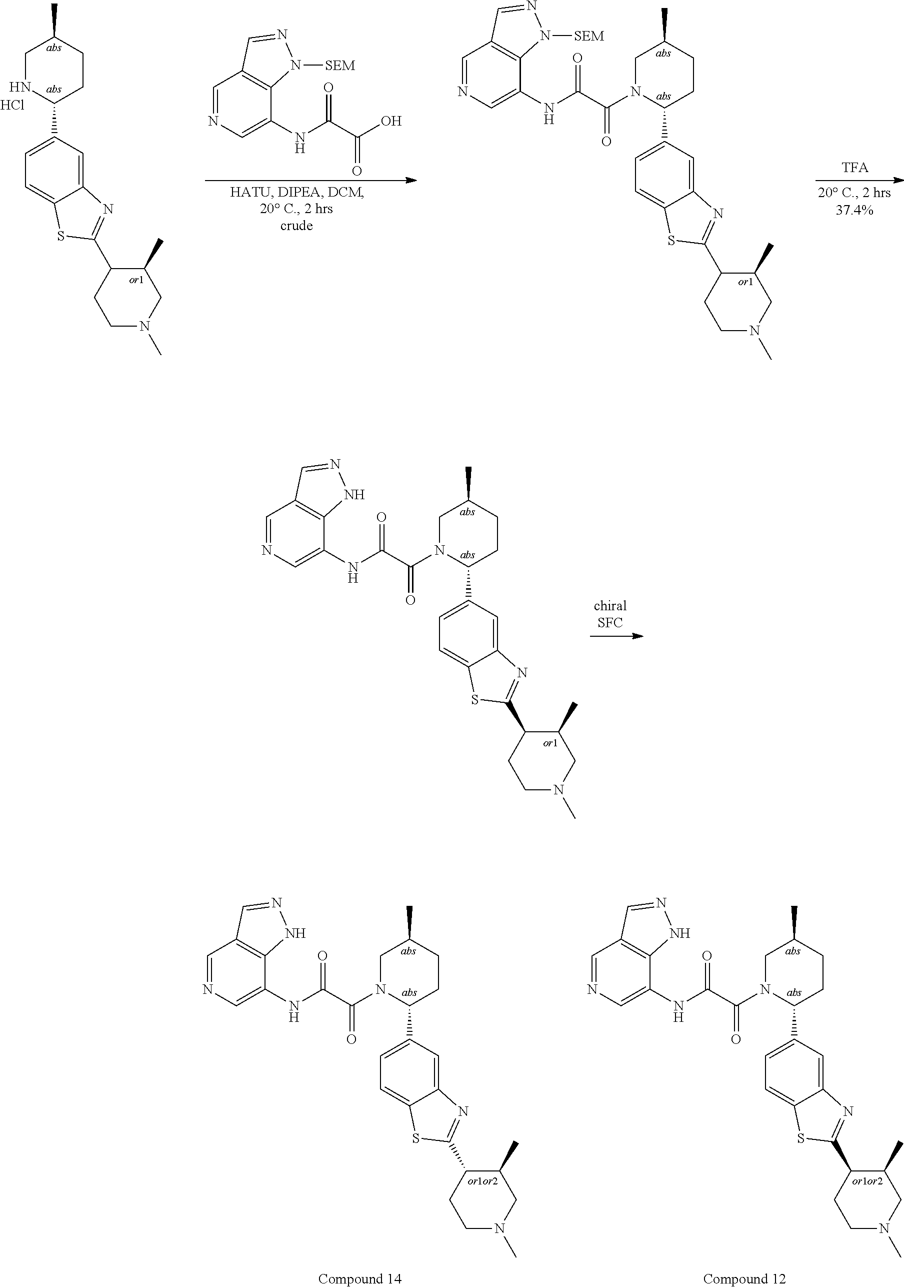

### Step 1: Synthesis of 2-oxo-2-[(2R,5S)-5-methyl-2-[2-[rel-(3R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide

To a solution of 2-[rel-(3R)-1,3-dimethyl-4-piperidyl]-5-[(2R,5S)-5-methyl-2-piperidyl]-1,3-benzothiazole (200 mg, 0.526 mmol, HCl), 2-oxo-2-[[1-(2-trimethylsilylethoxymethyl)pyrazolo[4,3-c]pyridin-7-yl]amino]acetic acid (Intermediate 37) (210 mg, 0.624 mmol), HATU (280 mg, 0.736 mmol) in DMF (6 mL) was added N-ethyl-N-isopropyl-propan-2-amine (400 μL, 2.30 mmol). The mixture was stirred at 20° C. for 2 hours. The resulting mixture was concentrated under reduced pressure. The residue was purified by flash chromatography (ISCO®; 12 g AgelaFlash® Silica Flash Column, DCM/MeOH with MeOH from 0˜10%, flow rate=30 mL/min, 254 nm) to afford 2-oxo-2-[(2R,5S)-5-methyl-2-[2-[rel-(3R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (250 mg, crude) as yellow oil. LCMS(ESI) [M+H]⁺ m/z: calcd 662.3, found 662.3.

### Step 2: Synthesis of 2-oxo-N-(1H-pyrazolo[4,3-c]pyridin-7-yl)-2-[(2R,5S)-5-methyl-2-[2-[rel-(3R)-1,3-dimethyl-4-piperidyl]-1,3-benzothiazol-5-yl]-1-piperidyl]acetamide

A mixture of 2-oxo-2-[(2R,5S)-5-methyl-2-[2-[rel-(3R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (200 mg, 0.302 mmol) and TFA (2 mL, 0.0259 mol) was stirred at 20° C. for 2 hours. The residue was purified by preparative HPLC (Instrument: Gilson GX-281 Liquid Handler, Gilson 322 Pump, Gilson 156 UV Detector; Column: Phenomenex C18 80×40 mm×3 μm; Mobile phase A: H₂O with 0.05% NH₃—H₂O (v %); Mobile phase B: MeCN; Gradient: B from 42% to 72% in 7.8 min, hold 100% B for 1 min; Flow Rate: 30 mL/min; Column Temperature: 30° C.; Wavelength: 220 nm, 254 nm) to afford 2-oxo-N-(1H-pyrazolo[4,3-c]pyridin-7-yl)-2-[(2R,5S)-5-methyl-2-[2-[rel-(3R)-1,3-dimethyl-4-piperidyl]-1,3-benzothiazol-5-yl]-1-piperidyl]acetamide (60 mg, 37.4% yield) as a white solid. ¹H NMR (400 MHZ, methanol-d₄) δ ppm 8.85-9.09 (m, 1H), 8.21-8.59 (m, 3H), 7.91-8.08 (m, 2H), 7.51 (br s, 1H), 5.62-5.98 (m, 1H), 3.87-4.22 (m, 1H), 3.44-3.79 (m, 3H), 2.70-3.11 (m, 4H), 2.11-2.63 (m, 5H), 1.84-2.08 (m, 2H), 1.49 (br d, J=11.3 Hz, 1H), 1.18 (br d, J=6.8 Hz, ³H), 0.95 (br d, J=6.8 Hz, ³H); LCMS(ESI) [M+H]⁺ m/z: calcd 532.2, found 532.1.

### Step 3: Synthesis of 2-oxo-N-(1H-pyrazolo[4,3-c]pyridin-7-yl)-2-[(2R,5S)-5-methyl-2-[2-[rel-(3R,4S)-1,3-dimethyl-4-piperidyl]-1,3-benzothiazol-5-yl]-1-piperidyl]acetamide (Compound 14) and 2-oxo-N-(1H-pyrazolo[4,3-c]pyridin-7-yl)-2-[(2R,5S)-5-methyl-2-[2-[rel-(3R,4R)-1,3-dimethyl-4-piperidyl]-1,3-benzothiazol-5-yl]-1-piperidyl]acetamide (Compound 12)

2-oxo-N-(1H-pyrazolo[4,3-c]pyridin-7-yl)-2-[(2R,5S)-5-methyl-2-[2-[rel-(3R)-1,3-dimethyl-4-piperidyl]-1,3-benzothiazol-5-yl]-1-piperidyl]acetamide (50 mg, 0.0941 mmol) was purified by chiral SFC (Instrument: Berger, MULTIGR AM-II; Column: Daicel Chiralpak AS 250×30 mm I.D. 10 μm; Mobile phase: supercritical CO₂/EtOH (0.1% NH₃H₂O, v %)=30/30; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to afford Compound 14 and Compound 12.

### Compound 14:

2-oxo-N-(1H-pyrazolo[4,3-c]pyridin-7-yl)-2-[(2R,5S)-5-methyl-2-[2-[rel-(3R,4S)-1,3-dimethyl-4-piperidyl]-1,3-benzothiazol-5-yl]-1-piperidyl]acetamide (12 mg, peak 1, Retention time=2.948 min, single unknown enantiomer with trans relative chemistry, white solid). 1H NMR (400 MHZ, methanol-d₄) δ ppm 8.82-9.09 (m, 1H), 8.27-8.56 (m, 2H), 7.90-8.10 (m, 2H), 7.50 (br s, 1H), 5.65-6.00 (m, 1H), 3.43-4.24 (m, 1H), 3.11 (br t, J=12.8 Hz, 2H), 2.84 (br d, J=8.3 Hz, 1H), 1.86-2.54 (m, 13H), 1.51 (br s, 1H), 1.25-1.36 (m, 1H), 1.18 (d, J=6.8 Hz, ³H), 0.81-0.92 (m, ³H); LCMS(ESI) [M+H]⁺ m/z: calcd 532.2, found 532.1; HPLC: 97.65%@254 nm; 97.2% ee.

### Compound 12:

2-oxo-N-(1H-pyrazolo[4,3-c]pyridin-7-yl)-2-[(2R,5S)-5-methyl-2-[2-[rel-(3R,4R)-1,3-dimethyl-4-piperidyl]-1,3-benzothiazol-5-yl]-1-piperidyl]acetamide (30 mg, peak 2, Retention time=3.350 min, single unknown enantiomer with trans relative chemistry, white solid). 1H NMR (400 MHz, methanol-d₄) δ ppm 8.84-9.10 (m, 1H), 8.25-8.61 (m, 2H), 7.93-8.09 (m, 2H), 7.50 (br d, J=6.9 Hz, 1H), 5.64-6.04 (m, 1H), 3.91-4.25 (m, 1H), 3.38-3.71 (m, 3H), 2.45-3.07 (m, 6H), 1.93-2.44 (m, 6H), 1.50 (br d, J=11.3 Hz, 1H), 1.15-1.33 (m, 4H), 0.83-1.04 (m, ³H); LCMS(ESI) [M+H]⁺ m/z: calcd 532.2, found 532.1; HPLC: 100.00%@254 nm; 97.8% ee.

### Example 77. Synthesis of 2-oxo-N-(1H-pyrazolo[4,3-c]pyridin-7-yl)-2-[(2R,5S)-5-methyl-2-[2-[rel-(3R,4S)-1,3-dimethyl-4-piperidyl]-1,3-benzothiazol-5-yl]-1-piperidyl]acetamide (Compound 14)

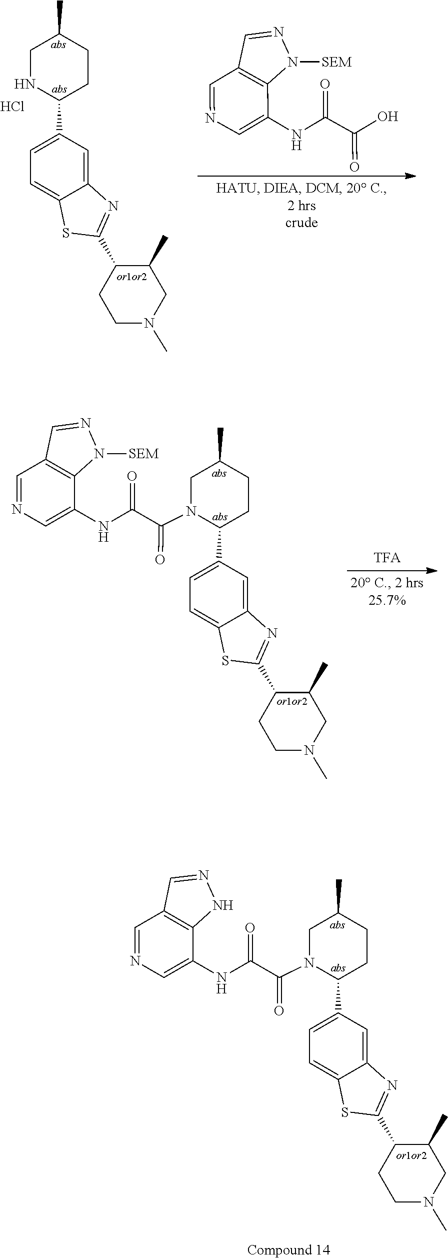

### Step 1: Synthesis of 2-oxo-2-[(2R,5S)-5-methyl-2-[2-[rel-(3R,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide

To a mixture of 2-oxo-2-[[1-(2-trimethylsilylethoxymethyl)pyrazolo[4,3-c]pyridin-7-yl]amino]acetic acid (Intermediate 37)(176 mg, 0.523 mmol) and 2-[rel-(3R,4S)-1,3-dimethyl-4-piperidyl]-5-[(2R,5S)-5-methyl-2-piperidyl]-1,3-benzothiazole (180 mg, 0.474 mmol, HCl) in DCM (5 mL) and DMF (2 mL) was added HATU (216 mg, 0.568 mmol) and DIPEA (0.25 mL, 1.44 mmol). The mixture was stirred at 20° C. for 2 hours. The resulting mixture was quenched by addition of saturated NH₄Cl aqueous solution (20 mL) and extracted with DCM (20 mL*3). The combined organic layer was dried over anhydrous Na₂SO₄, filtered and concentrated under reduced pressure to give 2-oxo-2-[(2R,5S)-5-methyl-2-[2-[rel-(3R,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (320 mg, crude) as brown oil, which was directly used without further purification. LCMS(ESI) [M+H]⁺ m/z: calcd 662.3; found 662.3.

### Step 2: Synthesis of 2-oxo-N-(1H-pyrazolo[4,3-c]pyridin-7-yl)-2-[(2R,5S)-5-methyl-2-[2-[rel-(3R,4S)-1,3-dimethyl-4-piperidyl]-1,3-benzothiazol-5-yl]-1-piperidyl]acetamide (Compound 14)

A mixture of 2-oxo-2-[(2R,5S)-5-methyl-2-[2-[rel-(3R,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (320 mg, 0.483 mmol) in TFA (2 mL) was stirred at 20° C. for 2 hours. The mixture was concentrated under reduced pressure. The residue was purified by preparative HPLC (Instrument: Gilson GX-281 Liquid Handler, Gilson 322 Pump, Gilson 156 UV Detector; Column: Phenomenex C18 80×40 mm×3 μm; Mobile phase A: H₂O with 10 mmol NH₄HCO₃ (v %); Mobile phase B: MeCN; Gradient: B from 35% to 65% in 7.8 min, hold 100% B for 2 min; Flow Rate: 30 mL/min; Column Temperature: 30° C.; Wavelength: 220 nm, 254 nm) to give 2-oxo-N-(1H-pyrazolo[4,3-c]pyridin-7-yl)-2-[(2R,5S)-5-methyl-2-[2-[rel-(3R,4S)-1,3-dimethyl-4-piperidyl]-1,3-benzothiazol-5-yl]-1-piperidyl]acetamide (66 mg, 25.7% yield) as white dry powder. ¹H NMR (400 MHZ, methanol-d₄) δ ppm 8.82-9.07 (m, 1H), 8.18-8.53 (m, 2H), 7.91-8.03 (m, 2H), 7.43-7.54 (m, 1H), 5.63-5.96 (m, 1H), 3.90-4.22 (m, 1H), 3.50 (d, J=11.3 Hz, 1H), 3.13-3.26 (m, 1H), 3.02 (t, J=12.5 Hz, 2H), 2.62-2.85 (m, 1H), 2.26-2.46 (m, 5H), 1.80-2.24 (m, 7H), 1.31-1.54 (m, 1H), 0.98-1.22 (m, 3H), 0.79-0.89 (m, ³H); LCMS(ESI) [M+H]⁺ m/z: calcd 532.2; found 532.2; HPLC: 94.70%@220 nm, 95.60%@254 nm; 100% ee.

### Example 78. Synthesis of 2-oxo-N-(1H-pyrazolo[4,3-c]pyridin-7-yl)-2-[(2R,5S)-5-methyl-2-[2-[rel-(3S,4R)-1,3-dimethyl-4-piperidyl]-1,3-benzothiazol-5-yl]-1-piperidyl]acetamide (Compound 71)

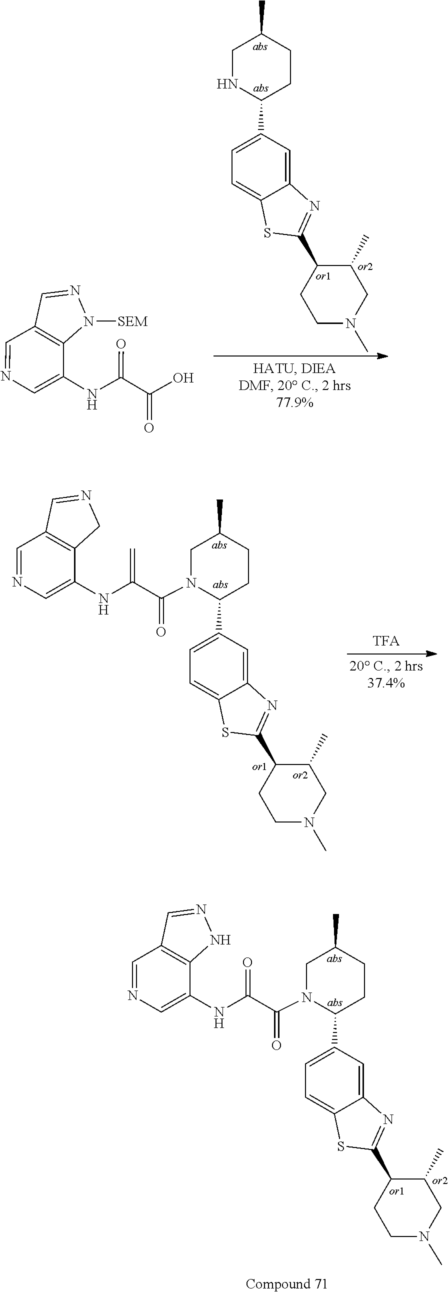 [SMILES: C[Si](C)(C)CCOCn1ncc2cncc(NC(=O)C(=O)O)c21 ; C[C@H]1CC[C@H](c2ccc3sc([C@@H]4CCN(C)C[C@H]4C)nc3c2)NC1 ; C=C(Nc1cncc2c1CN=C2)C(=O)N1C[C@@H](C)CC[C@@H]1c1ccc2sc([C@@H]3CCN(C)C[C@H]3C)nc2c1 ; C[C@H]1CC[C@H](c2ccc3sc([C@@H]4CCN(C)C[C@H]4C)nc3c2)N(C(=O)C(=O)Nc2cncc3cn[nH]c23)C1]

### Step 1: Synthesis of 2-oxo-2-[(2R,5S)-5-methyl-2-[2-[rel-(3S,4R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide

To a solution of 2-[rel-(3S,4R)-1,3-dimethyl-4-piperidyl]-5-[(2R,5S)-5-methyl-2-piperidyl]-1,3-benzothiazole (40 mg, 0.116 mmol), 2-oxo-2-[[1-(2-trimethylsilylethoxymethyl)pyrazolo[4,3-c]pyridin-7-yl]amino]acetic acid (Intermediate 37) (47 mg, 0.140 mmol) in DMF (4 mL) was added HATU (55 mg, 0.145 mmol) and N-ethyl-N-isopropyl-propan-2-amine (0.06 mL, 0.344 mmol). The mixture was stirred at 20° C. for 2 hours. The resulting mixture was concentrated under reduced pressure. The residue was purified by flash chromatography (ISCO®; 4 g AgelaFlash® Silica Flash Column, petroleum ether/EtOAc with EtOAc from 0˜15%, flow rate=30 mL/min, 254 nm) to afford 2-oxo-2-[(2R,5S)-5-methyl-2-[2-[rel-(3S,4R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (60 mg, 77.9% yield) as yellow oil. LCMS(ESI) [M+H]⁺ m/z: calcd 662.3, found 662.3.

### Step 2: Synthesis of 2-oxo-N-(1H-pyrazolo[4,3-c]pyridin-7-yl)-2-[(2R,5S)-5-methyl-2-[2-[rel-(3S,4R)-1,3-dimethyl-4-piperidyl]-1,3-benzothiazol-5-yl]-1-piperidyl]acetamide (Compound 71)

A mixture of 2-oxo-2-[(2R,5S)-5-methyl-2-[2-[rel-(3S,4R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (60 mg, 0.0906 mmol) and TFA (3 mL, 38.9 mmol) was stirred at 20° C. for 2 hours. The mixture was concentrated under reduced pressure. The residue was purified by preparative HPLC (Instrument: Gilson GX-281 Liquid Handler, Gilson 322 Pump, Gilson 156 UV Detector; Column: Phenomenex C18 80*40 mm*3 μm; Mobile phase A: H₂O with 0.05% NH₃—H₂O (v %); Mobile phase B: MeCN; Gradient: B from 25% to 55% in 9.5 min, hold 100% B for 2 min; Flow Rate: 25 mL/min; Column Temperature: 30° C.; Wavelength: 220 nm, 254 nm) to afford 2-oxo-N-(1H-pyrazolo[4,3-c]pyridin-7-yl)-2-[(2R,5S)-5-methyl-2-[2-[rel-(3S,4R)-1,3-dimethyl-4-piperidyl]-1,3-benzothiazol-5-yl]-1-piperidyl]acetamide (18 mg, 37.4% yield, single unknown enantiomer, white solid). ¹H NMR (400 MHZ, methanol-d₄) δ ppm 8.87-9.03 (m, 1H), 8.25-8.54 (m, 2H), 7.92-8.03 (m, 2H), 7.44-7.54 (m, 1H), 5.67-5.94 (m, 1H), 3.95 (br d, J=13.8 Hz, 1H), 3.50 (br d, J=14.3 Hz, 1H), 3.01-3.23 (m, 2H), 2.80 (br d, J=6.5 Hz, 1H), 2.39 (s, 5H), 2.22 (br d, J=11.0 Hz, 1H), 1.91-2.16 (m, 6H), 1.50 (br s, 1H), 1.17 (d, J=6.8 Hz, ³H), 0.79-0.91 (m, ³H);

LCMS(ESI) [M+H]⁺ m/z: calcd 532.2, found 532.2; HPLC: 99.38%@254 nm, 100%@254 nm; 91.6% ee.

### Example 79. Synthesis of 2-oxo-N-(1H-pyrazolo[4,3-c]pyridin-7-yl)-2-[(2R,5S)-5-methyl-2-[2-[rel-(3S,4R)-1,3-dimethyl-4-piperidyl]-1,3-benzothiazol-5-yl]-1-piperidyl]acetamide (Compound 71)

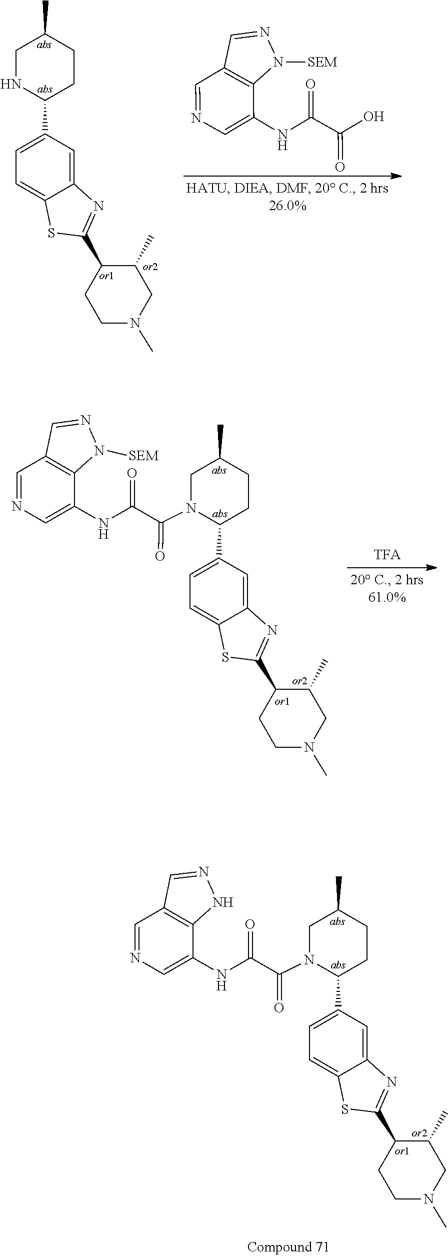

### Step 1: Synthesis of 2-oxo-2-[(2R,5S)-5-methyl-2-[2-[rel-(3S,4R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide

To a solution of 2-[rel-(3S,4R)-1,3-dimethyl-4-piperidyl]-5-[(2R,5S)-5-methyl-2-piperidyl]-1,3-benzothiazole (200 mg, 0.582 mmol), 2-oxo-2-[[1-(2-trimethylsilylethoxymethyl)pyrazolo[4,3-c]pyridin-7-yl]amino]acetic acid (Intermediate 37) (250 mg, 0.743 mmol) in DMF (4.9 mL) was added HATU (275 mg, 0.723 mmol) and N-ethyl-N-isopropyl-propan-2-amine (0.31 mL, 1.78 mmol). The mixture was stirred at 20° C. for 2 hours. The resulting mixture was concentrated under reduced pressure. The residue was purified by flash chromatography (ISCO®; 8 g AgelaFlash® Silica Flash Column, DCM/MeOH with MeOH from 0˜12%, flow rate=30 mL/min, 254 nm) to afford 2-oxo-2-[(2R,5S)-5-methyl-2-[2-[rel-(3S,4R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (100 mg, 26.0% yield) as yellow oil. LCMS(ESI) [M+H]⁺ m/z: calcd 662.3, found 662.3.

### Step 2: Synthesis of 2-oxo-N-(1H-pyrazolo[4,3-c]pyridin-7-yl)-2-[(2R,5S)-5-methyl-2-[2-[rel-(3S,4R)-1,3-dimethyl-4-piperidyl]-1,3-benzothiazol-5-yl]-1-piperidyl]acetamide (Compound 71)

A mixture of 2-oxo-2-[(2R,5S)-5-methyl-2-[2-[rel-(3S,4R)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (100 mg, 0.151 mmol) and TFA (3 mL, 0.0260 mol) was stirred at 20° C. for 2 hours. The mixture was concentrated under reduced pressure. The residue was purified by preparative HPLC (Instrument: Gilson GX-281 Liquid Handler, Gilson 322 Pump, Gilson 156 UV Detector; Column: Phenomenex C18 80*40 mm*3 μm; Mobile phase A: H₂O with NH₄HCO₃ (v %); Mobile phase B: MeCN; Gradient: B from 30% to 60% in 9.5 min, hold 100% B for 1 min; Flow Rate: 25 mL/min; Column Temperature: 30° C.; Wavelength: 220 nm, 254 nm) to afford 2-oxo-N-(1H-pyrazolo[4,3-c]pyridin-7-yl)-2-[(2R,5S)-5-methyl-2-[2-[rel-(3S,4R)-1,3-dimethyl-4-piperidyl]-1,3-benzothiazol-5-yl]-1-piperidyl]acetamide (49 mg, 61.0% yield) as white solid. ¹H NMR (400 MHZ, methanol-d₄) δ ppm 8.83-9.08 (m, 1H), 8.25-8.53 (m, 2H), 7.92-8.06 (m, 2H), 7.43-7.56 (m, 1H), 5.65-5.92 (m, 1H), 3.90-4.21 (m, 1H), 3.51 (br d, J=14.8 Hz, 1H), 3.01-3.24 (m, 2H), 2.75-2.87 (m, 1H), 2.22-2.48 (m, 5H), 1.88-2.21 (m, 7H), 1.50 (br s, 1H), 1.17 (br d, J=7.0 Hz, 3H), 0.79-0.93 (m, ³H); LCMS(ESI) [M+H]⁺ m/z: calcd 532.2, found 532.2; HPLC: 98.34%@220 nm, 98.57%@254 nm; 94.6% ee.

### Example 80. Synthesis of 2-oxo-N-(1H-pyrazolo[4,3-c]pyridin-7-yl)-2-[(2R,5S)-5-methyl-2-[2-[rel-(3S,4S)-1,3-dimethyl-4-piperidyl]-1,3-benzothiazol-5-yl]-1-piperidyl]acetamide (Compound 106)

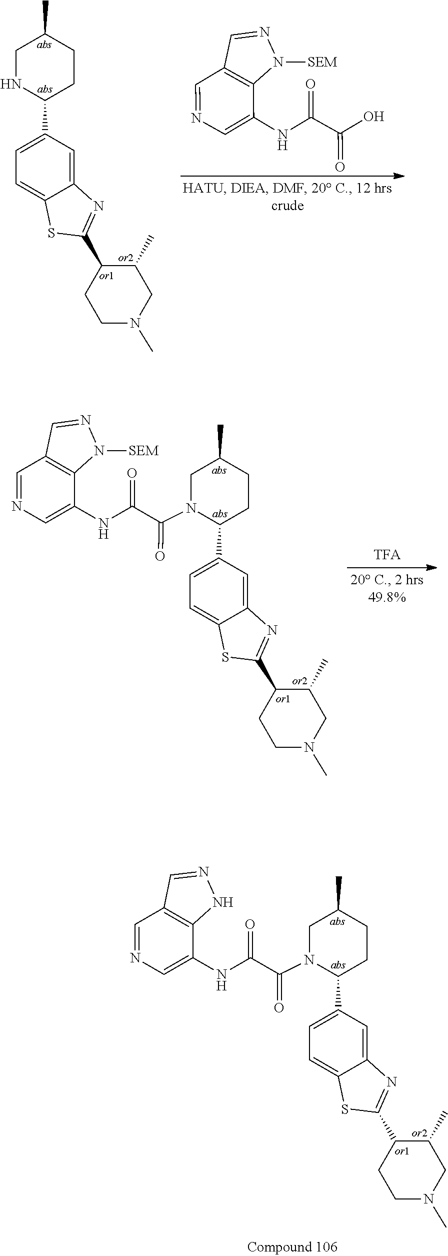

### Step 1: Synthesis of 2-oxo-2-[(2R,5S)-5-methyl-2-[2-[rel-(3S,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide

To a solution of 2-[rel-(3S,4S)-1,3-dimethyl-4-piperidyl]-5-[(2R,5S)-5-methyl-2-piperidyl]-1,3-benzothiazole (40 mg, 0.116 mmol), 2-oxo-2-[[1-(2-trimethylsilylethoxymethyl)pyrazolo[4,3-c]pyridin-7-yl]amino]acetic acid (Intermediate 37) (45 mg, 0.134 mmol), HATU (70 mg, 0.184 mmol) in DMF (4 mL) was added N-ethyl-N-isopropyl-propan-2-amine (0.06 mL, 0.344 mmol). The mixture was stirred at 20° C. for 12 hours. The resulting mixture was concentrated under reduced pressure. The residue was purified by flash chromatography (ISCO®; 4 g AgelaFlash® Silica Flash Column, DCM/MeOH with MeOH from 0˜10%, flow rate=30 mL/min, 254 nm) to afford 2-oxo-2-[(2R,5S)-5-methyl-2-[2-[rel-(3S,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (60 mg, crude) as yellow oil. LCMS(ESI) [M+H]⁺ m/z: calcd 662.3, found 662.3.

### Step 2: Synthesis of 2-oxo-N-(1H-pyrazolo[4,3-c]pyridin-7-yl)-2-[(2R,5S)-5-methyl-2-[2-[rel-(3S,4S)-1,3-dimethyl-4-piperidyl]-1,3-benzothiazol-5-yl]-1-piperidyl]acetamide (Compound 106)

A mixture of 2-oxo-2-[(2R,5S)-5-methyl-2-[2-[rel-(3S,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (50 mg, 0.0755 mmol) and TFA (2 mL) was stirred at 20° C. for 2 hours. The resulting mixture was concentrated under reduced pressure. The residue was purified by flash chromatography (Biotage®, Column: SepaFlash® Sphercial C18, 40 g, 40-60 μm, 120 Å; MeCN/water (0.05% NH₃—H₂O) with MeCN from 0˜35%, 30 mL/min, 254 nm) to afford 2-oxo-N-(1H-pyrazolo[4,3-c]pyridin-7-yl)-2-[(2R,5S)-5-methyl-2-[2-[rel-(3S,4S)-1,3-dimethyl-4-piperidyl]-1,3-benzothiazol-5-yl]-1-piperidyl]acetamide (20 mg, 49.8% yield) as white solid. ¹H NMR (400 MHZ, methanol-d₄) δ ppm 8.84-9.08 (m, 1H), 8.35-8.53 (m, 1H), 8.28 (br d, J=14.6 Hz, 1H), 7.89-8.06 (m, 2H), 7.48 (br s, 1H), 5.62-5.97 (m, 1H), 3.92-4.20 (m, 1H), 3.37-3.58 (m, 1H), 2.93-3.09 (m, 1H), 2.83 (br s, 1H), 2.18-2.56 (m, 9H), 1.91-2.11 (m, 3H), 1.51 (br s, 1H), 1.11-1.36 (m, 4H), 0.79-1.00 (m, ³H); LCMS(ESI) [M+H]⁺ m/z: calcd 532.2, found 532.3; HPLC: 99.54%@220 nm, 100%@254 nm.

### Example 81. The Synthesis of 2-oxo-N-(1H-pyrazolo[4,3-c]pyridin-7-yl)-2-[(2R,5S)-5-methyl-2-[2-[rel-(3S,4S)-1,3-dimethyl-4-piperidyl]-1,3-benzothiazol-5-yl]-1-piperidyl]acetamide (Compound 95)

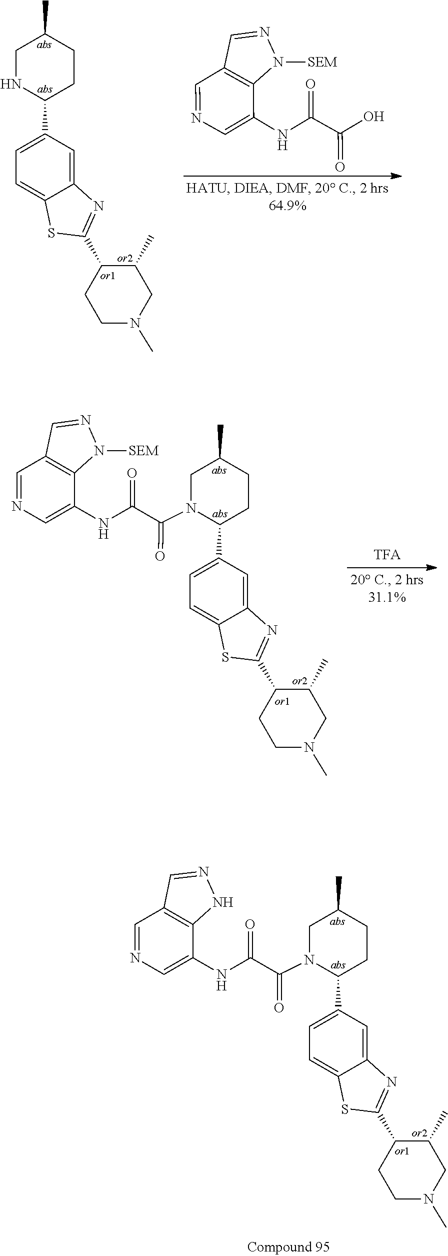

### Step 1: Synthesis of 2-oxo-2-[(2R,5S)-5-methyl-2-[2-[rel-(3S,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide

To a solution of 2-[rel-(3S,4S)-1,3-dimethyl-4-piperidyl]-5-[(2R,5S)-5-methyl-2-piperidyl]-1,3-benzothiazole (200 mg, 0.582 mmol), 2-oxo-2-[[1-(2-trimethylsilylethoxymethyl)pyrazolo[4,3-c]pyridin-7-yl]amino]acetic acid (Intermediate 37) (240 mg, 0.713 mmol), HATU (300 mg, 0.789 μmol) in DMF (6 mL) was added N-ethyl-N-isopropyl-propan-2-amine (300 μL, 1.72 mmol). The mixture was stirred at 20° C. for 2 hours. The resulting mixture was concentrated under reduced pressure. The residue was purified by flash chromatography (ISCO®; 12 g AgelaFlash® Silica Flash Column, DCM/MeOH with MeOH from 0˜10%, flow rate=30 mL/min, 254 nm) to afford 2-oxo-2-[(2R,5S)-5-methyl-2-[2-[rel-(3S,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (250 mg, 64.9% yield) as yellow solid. ¹H NMR (400 MHZ, methanol-d₄) δ ppm 9.01 (br s, 1H), 8.69 (d, J=3.5 Hz, 1H), 8.23-8.58 (m, 2H), 7.94-8.12 (m, 1H), 7.49 (dd, J=8.5, 4.5 Hz, 1H), 5.93 (br s, 1H), 3.78-4.33 (m, 1H), 3.43-3.71 (m, 5H), 3.12-3.27 (m, 2H), 2.91-3.04 (m, 2H), 2.15-2.70 (m, 4H), 1.84-2.11 (m, 2H), 1.44-1.61 (m, 1H), 1.34-1.41 (m, 10H), 1.20 (br d, J=7.0 Hz, 2H), 0.74-1.10 (m, 4H), −0.18-0.09 (m, 6H); LCMS(ESI) [M+H]⁺ m/z: calcd 662.3, found 662.3.

### Step 2: Synthesis of 2-oxo-N-(1H-pyrazolo[4,3-c]pyridin-7-yl)-2-[(2R,5S)-5-methyl-2-[2-[rel-(3S,4S)-1,3-dimethyl-4-piperidyl]-1,3-benzothiazol-5-yl]-1-piperidyl]acetamide (Compound 95)

A mixture of 2-oxo-2-[(2R,5S)-5-methyl-2-[2-[rel-(3S,4S)-1,3-dimethyl-4-piperidyl]-1,3-benzothiazol-5-yl]-1-piperidyl]-N-[1-(2-trimethylsilylethoxymethyl)pyrazolo[4,3-c]pyridin-7-yl]acetamide (200 mg, 0.302 mmol) and TFA (4 mL, 0.0519 mol) was stirred at 20° C. for 2 hours. The resulting mixture was concentrated under reduced pressure. The residue was purified by preparative HPLC (Instrument: (Instrument: Gilson GX-281 Liquid Handler, Gilson 322 Pump, Gilson 156 UV Detector; Column: Phenomenex C18 80×40 mm×3 μm; Mobile phase A: H₂O with 10 mm NH₄HCO₃ (v %); Mobile phase B: MeCN; Gradient: B from 36% to 66% in 7.8 min, hold 100% B for 1 min; Flow Rate: 25 mL/min; Column Temperature: 30° C.; Wavelength: 220 nm, 254 nm) to afford to afford 2-oxo-N-(1H-pyrazolo[4,3-c]pyridin-7-yl)-2-[(2R,5S)-5-methyl-2-[2-[rel-(3S,4S)-1,3-dimethyl-4-piperidyl]-1,3-benzothiazol-5-yl]-1-piperidyl]acetamide (50 mg, 31.1% yield) as a white solid. ¹H NMR (400 MHZ, methanol-d₄) δ ppm 8.83-9.08 (m, 1H), 8.22-8.52 (m, 2H), 7.88-8.08 (m, 2H), 7.48 (br d, J=7.4 Hz, 1H), 5.61-5.95 (m, 1H), 3.91-4.21 (m, 1H), 3.37-3.65 (m, 2H), 3.08 (br s, 1H), 2.91 (br s, 1H), 2.18-2.65 (m, 10H), 1.90-2.16 (m, 3H), 1.50 (br d, J=10.1 Hz, 1H), 1.18 (br d, J=6.9 Hz, ³H), 0.86-0.99 (m, ³H); LCMS (ESI) [M+H]⁺ m/z: calcd 532.3, found 532.3; HPLC: 100%@254 nm; 95.6% ee.

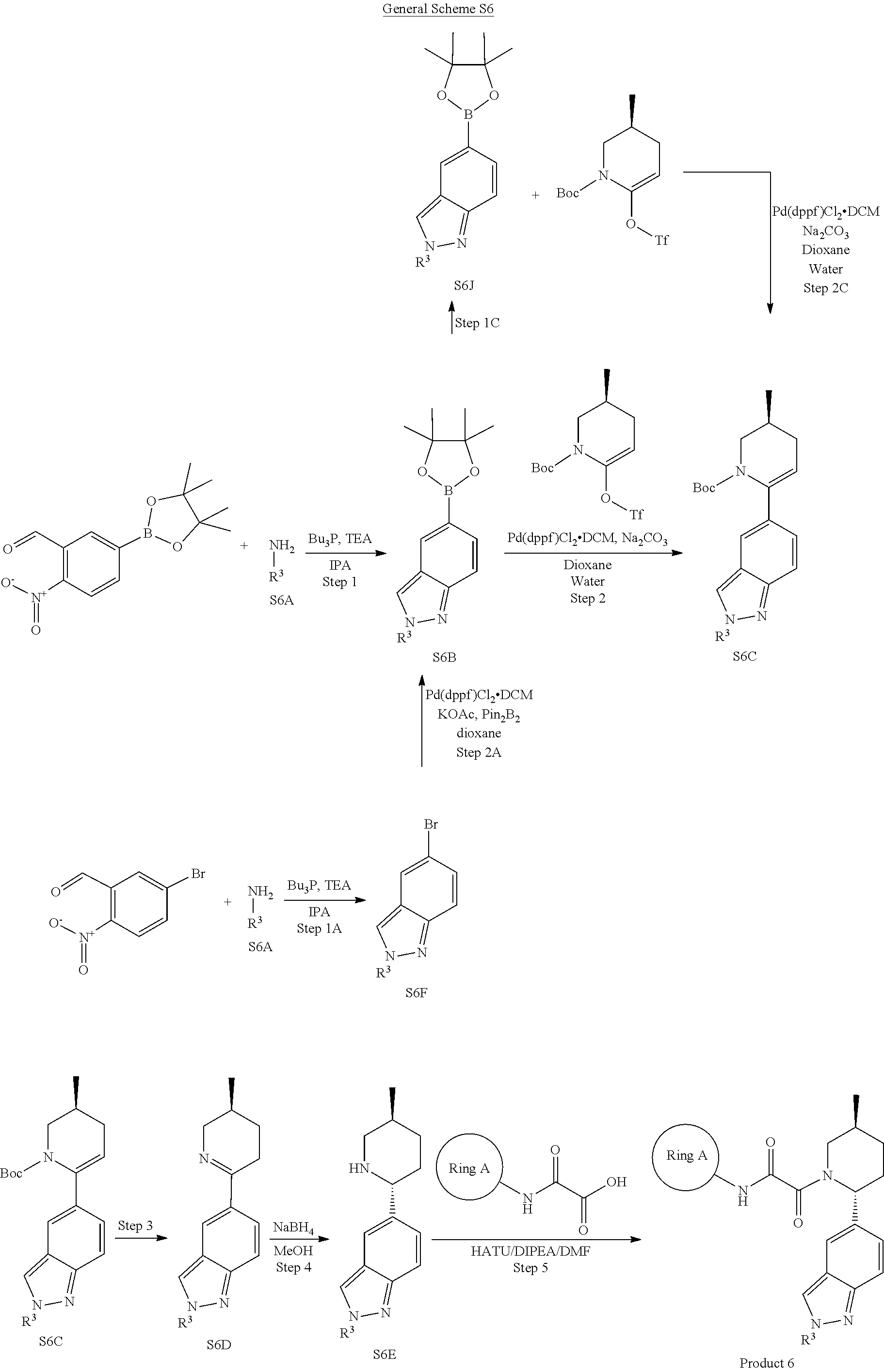

### Step 1: General Procedure for S6B

2-Nitro-5-(4,4,5,5-tetramethyl-1,3,2-dioxaborolan-2-yl)benzaldehyde (1.0 equiv.) and S6A (1.0 equiv., or it's salt) were dissolved in i-PrOH (140 mL). The resulting mixture was stirred at 80° C. for 2 hours following by the addition of tri-n-butyl phosphine (Bu₃P, 3.0 equiv.). The reaction mixture was stirred under reflux for 2 hours. Then, the volatiles were removed in vacuo. The residue was dissolved in DCM and washed with water. The organic layer separated, dried over Na₂SO₄ and concentrated under reduced pressure. The residue was purified by flash chromatography to afford S6B.

### Step 1A: General Procedure for S6F

5-Bromo-2-nitro-benzaldehyde (1.0 equiv.) and S6A (1.0 equiv.) were dissolved in i-PrOH (400.0 mL). The resulting mixture was stirred at 80° C. for 2 hr following by the addition of tri-n-butyl phosphine (3.0 equiv.). The reaction mixture was refluxed additionally for 16 hr. Then the reaction mixture was evaporated under reduced pressure and purified by flash chromatography to afford S6F.

### Step 1C: General Procedure for S6J

Potassium carbonate, anhydrous, 99% (3.0 equiv.) and iodomethane (1.7 equiv) were added to the solution of S6B (1.0 equiv.) in MeCN (appr. 45.0 mL). The resulting mixture was stirred at 25° C. for 16 hr. Then, solvent was removed under reduced pressure. The residue was diluted with water (40.0 mL) and the resulting mixture was extracted with DCM to S6J.

### Step 2: General Procedure for S6C

S6B (1.0 equiv.), tert-butyl rac-(3S)-3-methyl-6-(trifluoromethylsulfonyloxy)-3,4-dihydro-2H-pyridine-1-carboxylate (1.01 equiv.), sodium carbonate (3.0 equiv.) and Pd(dppf)Cl₂ DCM (0.05 equiv.) were stirred in a mixture of 1,4-dioxane (6.0 mL) and water (2.0 mL) under inert atmosphere at 85° C. for 15 hr. Upon completion, the reaction mixture was cooled down, diluted with water and extracted with DCM. The organic layer was separated, dried over Na₂SO₄ and concentrated under reduced pressure to afford S6C. The obtained material was used in the next step without an additional purification.

### Step 2A: General Procedure for S6B

S6F (1.0 equiv.), B₂Pin₂ (1.1 equiv.) and Potassium Acetate (2.0 equiv) were mixed together in 1,4-dioxane (appr. 20.0 mL). The resulting mixture was evacuated and then backfilled with argon, this operation was repeated three times, then Pd(dppf)₂Cl₂ DCM (993.01 mg, 1.22 mmol) was added under argon. The reaction mixture was stirred under argon atmosphere at 90° C. for 14 hr. Then the mixture was allowed to cool to the room temperature and the volatiles were removed in vacuo to afford S6B.

### Step 2C: General Procedure for S6

Sodium carbonate (2.0 equiv.) was added to a solution of S6J (1.0 equiv.) and tert-butyl rac-(5S)-5-methyl-2-(trifluoromethylsulfonyloxy)piperidine-1-carboxylate (1.1 equiv.) in Water (appr. 15.0 mL) and 1,4-dioxane (appr. 50.0 mL). Reaction flask was evacuated and refilled with argon 3 times. Then, Pd(dppf) Cl₂·CH₂Cl₂ (0.05 equiv.) was added under stream of argon. The resulting mixture was stirred at 90° C. for 15 hr under inert atmosphere. Upon completion of the reaction, water (appr. 50.0 mL) was added. The resulting mixture was extracted with EtOAc (3+20.0 mL). The combined organic layers were dried over Na₂SO₄, filtered and concentrated in vacuo to obtain S6C. The obtained material was used in the next step without an additional purification.

### Step 3: General Procedure for S6D

### Method A

S6C (1.0 equiv.) was dissolved in a mixture of TFA (1.0 mL) and DCM (1.0 mL). The reaction mixture was stirred at room temperature for 30 minutes. Then, the mixture was concentrated in vacuo to S6D. The obtained material was used in the next step without an additional purification.

### Method B

S6C (1.0 equiv.) was dissolved in a mixture of HCl (4.0M solution in dioxane, 1.0 mL) and MeOH (1.0 mL). The reaction mixture was stirred at room temperature overnight. Then, the mixture was concentrated in vacuo to S6D. The obtained material was used in the next step without an additional purification.

### Step 4: General Procedure for S6E

Sodium Borohydride (2.0 equiv) was portionwise added to the solution of S6D (1.0 equiv.) in MeOH (5.0 mL). The reaction mixture was stirred at room temperature for 17 hours. Then, the mixture was acidified with HCl (4.0M solution in dioxane) to pH 5 and the volatiles were removed in vacuo to afford S6E. The obtained material was used in the next step without an additional purification.

### Step 5: General Procedure for Product 6

HATU (1.4 equiv.) was added to the stirred solution of S6E (1.0 equiv), corresponding acid (1.1 equiv) and DIPEA (10.0 equiv) in DMSO (appr. 6.0 mL). The resulting reaction mixture was stirred at 25° C. for 4 hr. Upon completion, the reaction mixture was submitted to reverse phase HPLC to afford Product 6.

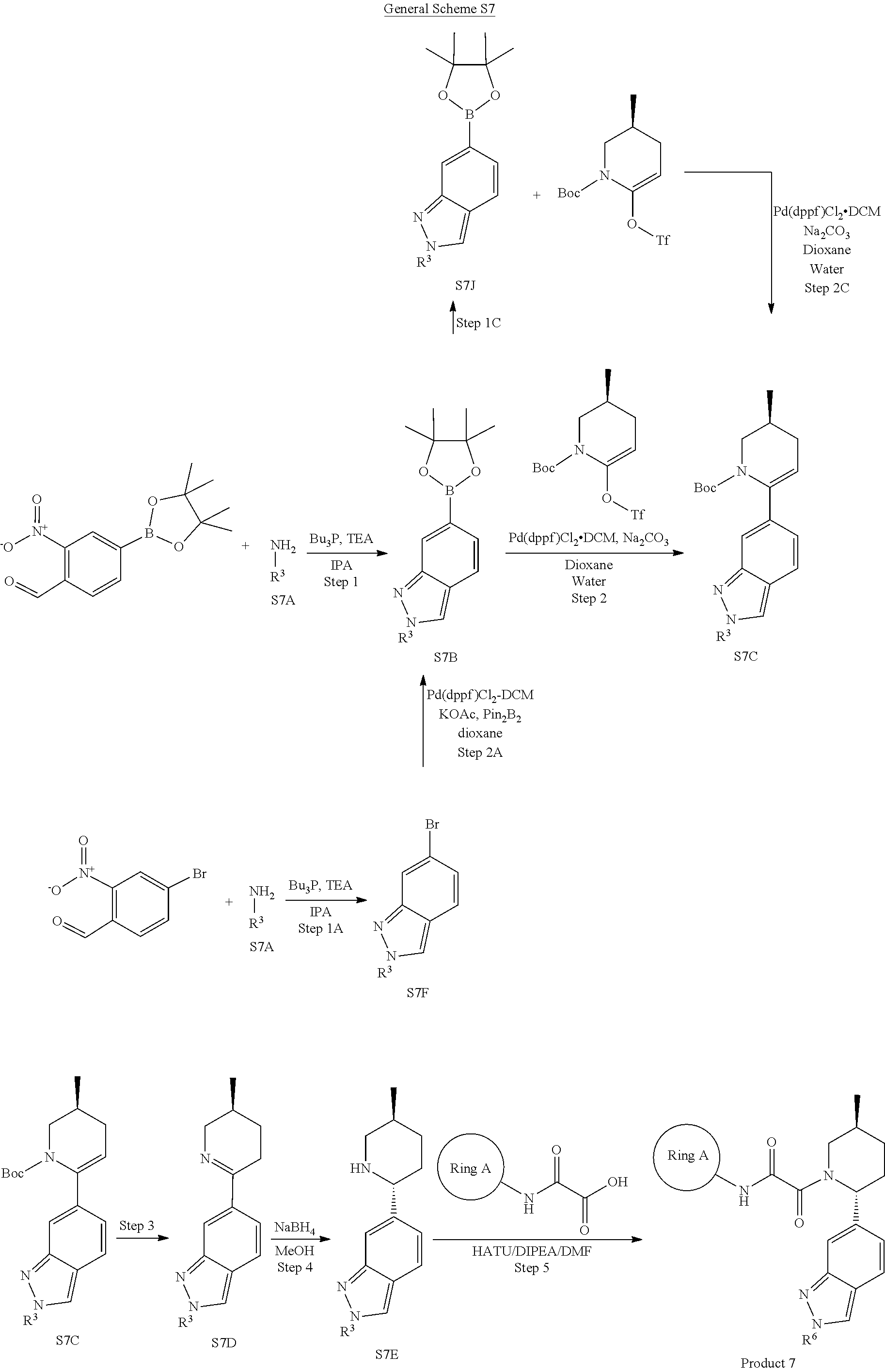

### Step 1: General Procedure for S7B

2-Nitro-5-(4,4,5,5-tetramethyl-1,3,2-dioxaborolan-2-yl)benzaldehyde (1.0 equiv.) and S7A (1.0 equiv., or it's salt) were dissolved in i-PrOH (140 mL). The resulting mixture was stirred at 80° C. for 2 hours following by the addition of tri-n-butyl phosphine (Bu₃P, 3.0 equiv.). The reaction mixture was stirred under reflux for 2 hours. Then, the volatiles were removed in vacuo. The residue was dissolved in DCM and washed with water. The organic layer separated, dried over Na₂SO₄ and concentrated under reduced pressure. The residue was purified by flash chromatography to afford S7B.

### Step 1A: General Procedure for S7F

5-Bromo-2-nitro-benzaldehyde (1.0 equiv.) and S7A (1.0 equiv.) were dissolved in i-PrOH (400.0 mL). The resulting mixture was stirred at 80° C. for 2 hr following by the addition of tri-n-butyl phosphine (3.0 equiv.). The reaction mixture was refluxed additionally for 16 hr. Then the reaction mixture was evaporated under reduced pressure and purified by flash chromatography to afford S7F.

### Step 1C: General Procedure for S7J

Potassium carbonate, anhydrous, 99% (3.0 equiv.) and iodomethane (1.7 equiv) were added to the solution of S7B (1.0 equiv.) in MeCN (appr. 45.0 mL). The resulting mixture was stirred at 25° C. for 16 hr. Then, solvent was removed under reduced pressure. The residue was diluted with water (40.0 mL) and the resulting mixture was extracted with DCM to S7J.

### Step 2: General Procedure for S7C

S7B (1.0 equiv.), tert-butyl rac-(3S)-3-methyl-6-(trifluoromethylsulfonyloxy)-3,4-dihydro-2H-pyridine-1-carboxylate (1.01 equiv.), sodium carbonate (3.0 equiv.) and Pd(dppf)Cl₂ DCM (0.05 equiv.) were stirred in a mixture of 1,4-dioxane (6.0 mL) and water (2.0 mL) under inert atmosphere at 85° C. for 15 hr. Upon completion, the reaction mixture was cooled down, diluted with water and extracted with DCM. The organic layer was separated, dried over Na₂SO₄ and concentrated under reduced pressure to afford S7C. The obtained material was used in the next step without an additional purification.

### Step 2A: General Procedure for S7B

S7F (1.0 equiv.), B₂Pin₂ (1.1 equiv.) and Potassium Acetate (2.0 equiv) were mixed together in 1,4-dioxane (appr. 20.0 mL). The resulting mixture was evacuated and then backfilled with argon, this operation was repeated three times, then Pd(dppf)₂Cl₂ DCM (993.01 mg, 1.22 mmol) was added under argon. The reaction mixture was stirred under argon atmosphere at 90° C. for 14 hr. Then the mixture was allowed to cool to the room temperature and the volatiles were removed in vacuo to afford S7B.

### Step 2C: General Procedure for S7C

Sodium carbonate (2.0 equiv.) was added to a solution of S7J (1.0 equiv.) and tert-butyl rac-(5S)-5-methyl-2-(trifluoromethylsulfonyloxy)piperidine-1-carboxylate (1.1 equiv.) in Water (appr. 15.0 mL) and 1,4-dioxane (appr. 50.0 mL). Reaction flask was evacuated and refilled with argon 3 times. Then, Pd(dppf)Cl₂·CH₂Cl₂ (0.05 equiv.) was added under stream of argon. The resulting mixture was stirred at 90° C. for 15 hr under inert atmosphere. Upon completion of the reaction, water (appr. 50.0 mL) was added. The resulting mixture was extracted with EtOAc (3*20.0 mL). The combined organic layers were dried over Na₂SO₄, filtered and concentrated in vacuo to obtain S7C. The obtained material was used in the next step without an additional purification.

### Step 3: General Procedure for S7D

### Method A

S7C (1.0 equiv.) was dissolved in a mixture of TFA (1.0 mL) and DCM (1.0 mL). The reaction mixture was stirred at room temperature for 30 minutes. Then, the mixture was concentrated in vacuo to S7D. The obtained material was used in the next step without an additional purification.

### Method B

S7C (1.0 equiv.) was dissolved in a mixture of HCl (4.0M solution in dioxane, 1.0 mL) and MeOH (1.0 mL). The reaction mixture was stirred at room temperature overnight. Then, the mixture was concentrated in vacuo to S7D. The obtained material was used in the next step without an additional purification.

### Step 4: General Procedure for S7E

Sodium Borohydride (2.0 equiv) was portionwise added to the solution of S7D (1.0 equiv.) in MeOH (5.0 mL). The reaction mixture was stirred at room temperature for 17 hours. Then, the mixture was acidified with HCl (4.0M solution in dioxane) to pH 5 and the volatiles were removed in vacuo to afford S7E. The obtained material was used in the next step without an additional purification.

### Step 5: General Procedure for Product 7

HATU (1.4 equiv.) was added to the stirred solution of S7E (1.0 equiv), corresponding acid (1.1 equiv) and DIPEA (10.0 equiv) in DMSO (appr. 6.0 mL). The resulting reaction mixture was stirred at 25° C. for 4 hr. The resulting mixture was submitted to reverse phase HPLC to afford Product 7.

### Example 82. Synthesis of 2-oxo-N-(1H-pyrazolo[4,3-c]pyridin-7-yl)-2-[(2R,5S)-5-methyl-2-[2-(1,2,2,6,6-pentamethyl-4-piperidyl) indazol-5-yl]-1-piperidyl]acetamide (Compound 47)

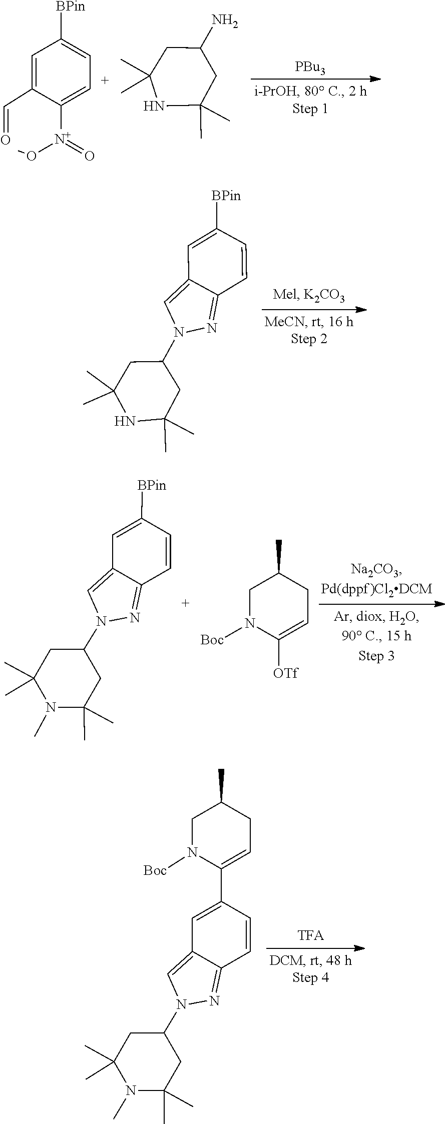

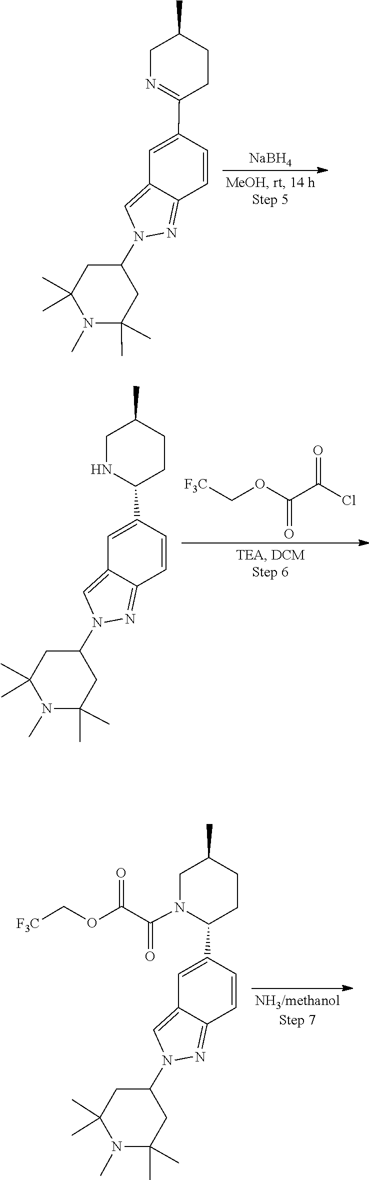

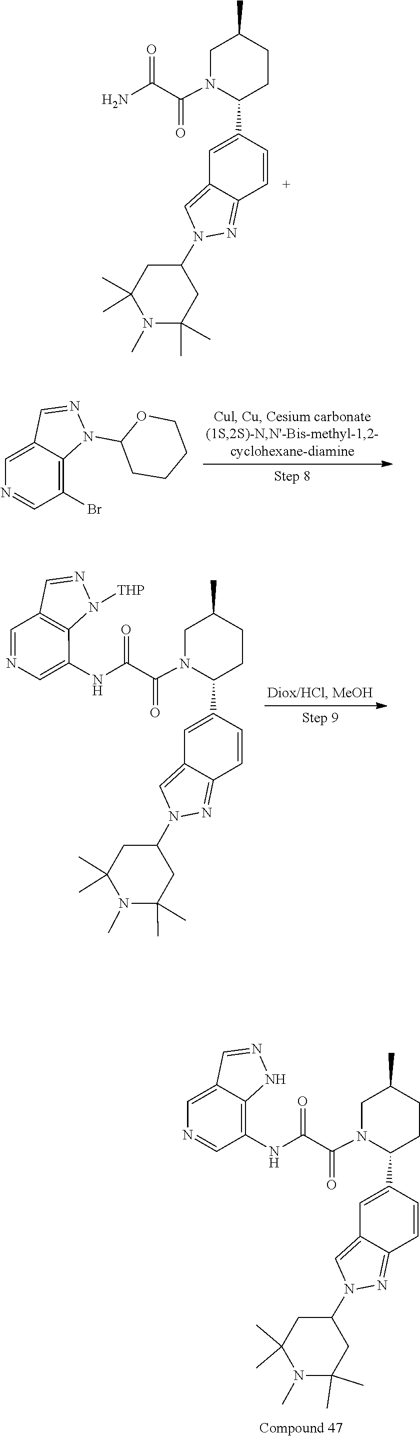

### Step 1: The Synthesis of 5-(4,4,5,5-Tetramethyl-1,3,2-dioxaborolan-2-yl)-2-(2,2,6,6-tetramethyl-4-piperidyl)indazole

### Prepared by General Procedure Scheme S6 Step 1

Yield: 2.8 g (crude); LCMS(ESI): [M+H]⁺ m/z: calcd 384.2; found 384.2; Rt=1.133 min.

### Step 2: The Synthesis of 2-(1,2,2,6,6-Pentamethyl-4-piperidyl)-5-(4,4,5,5-tetramethyl-1,3,2-dioxaborolan-2-yl)indazole

### Prepared by General Procedure Scheme S6 Step 1C

Yield: 3.0 g (crude); LCMS(ESI): [M+H]⁺ m/z: calcd 398.2; found 398.1; Rt=1.152 min.

### Step 3: The Synthesis of tert-Butyl (3S)-3-methyl-6-[2-(1,2,2,6,6-pentamethyl-4-piperidyl) indazol-5-yl]-3,4-dihydro-2H-pyridine-1-carboxylate

### Prepared by General Procedure Scheme S6 Step 2C

Yield: 3.5 g (crude); LCMS(ESI): [M+H]⁺ m/z: calcd 467.2; found 467.2; Rt=1.232 min.

### Step 4: The Synthesis of 2-(1,2,2,6,6-Pentamethyl-4-piperidyl)-5-[(3S)-3-methyl-1,2,3,4-tetrahydropyridin-6-yl]indazole

### Prepared by General Procedure Scheme S6 Step 3 (Method A)

Yield: 1.1 g (crude); LCMS(ESI): [M+H]⁺ m/z: calcd 367.2; found 367.2; Rt=0.725 min.

### Step 5: The Synthesis of 2-(1,2,2,6,6-Pentamethyl-4-piperidyl)-5-[(2R,5S)-5-methyl-2-piperidyl]indazole

### Prepared by General Procedure Scheme S6 Step 4

Yield: 1.3 g (crude); LCMS(ESI): [M+H]⁺ m/z: calcd 369.2; found 369.2; Rt=0.756 min.

### Step 6: The Synthesis of 2,2,2-trifluoroethyl 2-oxo-2-[(2R,5S)-5-methyl-2-[2-(1,2,2,6,6-pentamethyl-4-piperidyl) indazol-5-yl]-1-piperidyl]acetate

2-(1,2,2,6,6-pentamethyl-4-piperidyl)-5-[(2R,5S)-5-methyl-2-piperidyl]indazole (460.00 mg, 1.25 mmol) and TEA (189.44 mg, 1.87 mmol, 260.94 μL) were mixed together in DCM (15.02 mL) and the resulting solution was cooled to 5° C. in an ice bath. 2,2,2-trifluoroethyl 2-chloro-2-oxo-acetate (261.55 mg, 1.37 mmol) was added dropwise to the previous solution and the resulting mixture was allowed to warm to rt and stirred overnight. Upon completion of the reaction, the mixture was washed with brine (2*20 ml), organic layer was dried over Na₂SO₄, filtered and concentrated in vacuo to affording 2,2,2-trifluoroethyl 2-oxo-2-[(2R,5S)-5-methyl-2-[2-(1,2,2,6,6-pentamethyl-4-piperidyl) indazol-5-yl]-1-piperidyl]acetate (0.5 g, crude);

LCMS(ESI): [M+H]⁺ m/z: calcd 522.3; found 523.4; Rt=1.190 min.

### Step 6: The Synthesis of 2-oxo-2-[(2R,5S)-5-methyl-2-[2-(1,2,2,6,6-pentamethyl-4-piperidyl) indazol-5-yl]-1-piperidyl]acetamide

2,2,2-trifluoroethyl 2-oxo-2-[(2R,5S)-5-methyl-2-[2-(1,2,2,6,6-pentamethyl-4-piperidyl) indazol-5-yl]-1-piperidyl]acetate (500.00 mg, 956.75 μmol) was dissolved in NH₃/MeOH (10 mL) and the resulting mixture was stirred for 18 hr at 25° C. Upon completion the resulting mixture was evaporated to dryness to afford crude product.

LCMS(ESI): [M+H]⁺ m/z: calcd 439.3; found 440.4; Rt=0.942 min.

### Step 7: The Synthesis of 2-oxo-2-[(2R,5S)-5-methyl-2-[2-(1,2,2,6,6-pentamethyl-4-piperidyl) indazol-5-yl]-1-piperidyl]-N-(1-tetrahydropyran-2-ylpyrazolo[4,3-c]pyridin-7-yl)acetamide

2-oxo-2-[(2R,5S)-5-methyl-2-[2-(1,2,2,6,6-pentamethyl-4-piperidyl) indazol-5-yl]-1-piperidyl]acetamide (200.00 mg, 454.97 μmol), 7-bromo-1-tetrahydropyran-2-yl-pyrazolo[4,3-c]pyridine (128.36 mg, 454.97 μmol), Cu (28.91 mg, 454.97 μmol), CuI (86.65 mg, 454.97 μmol, 15.42 μL), Cesium carbonate (296.47 mg, 909.93 μmol) and (1S,2S)—N,N′-Bis-methyl-1,2-cyclohexane-diamine (97.07 mg, 682.45 μmol, 107.62 μL) were mixed in Dioxane (4 mL) under argon. The resulting mixture was allowed to stir at 100° C. for 15 hr in vial. Upon completion of the reaction, dioxane was evaporated and residue was subjected by HPLC (column: XBridge C18 100×19 mm, 5 um; mobile phase: 20-65% 0-6 min H₂O/MeOH/0.1% NH₄OH, flow rate: 30 ml/min (loading pump 4 ml/min methanol), affording 2-oxo-2-[(2R,5S)-5-methyl-2-[2-(1,2,2,6,6-pentamethyl-4-piperidyl) indazol-5-yl]-1-piperidyl]-N-(1-tetrahydropyran-2-ylpyrazolo[4,3-c]pyridin-7-yl)acetamide (42.4 mg, 66.17 μmol, 14.54% yield) on two portions. LCMS(ESI): [M+H]⁺ m/z: calcd 640.8; found 640.1); Rt=1.840 min.

### Step 8: The Synthesis of 2-oxo-N-(1H-pyrazolo[4,3-c]pyridin-7-yl)-2-[(2R,5S)-5-methyl-2-[2-(1,2,2,6,6-pentamethyl-4-piperidyl) indazol-5-yl]-1-piperidyl]acetamide (Compound 47)

2-oxo-2-[(2R,5S)-5-methyl-2-[2-(1,2,2,6,6-pentamethyl-4-piperidyl) indazol-5-yl]-1-piperidyl]-N-(1-tetrahydropyran-2-ylpyrazolo[4,3-c]pyridin-7-yl)acetamide (42.40 mg, 66.17 μmol) was dissolved in a mixture of Diox/HCl (1 mL) and MeOH (1 mL), the resulting mixture was stirred at 25° C. for 16 hr. After that time, solvent was evaporated to dryness to afford crude product, then crude product was subjected by HPLC (column: Chromatorex 18 SMB100-5T 100×19 mm, 5 um; mobile phase: 0-0-30% 0-1-6 min H₂O/MeCN/0.1% FA, flow rate: 30 ml/min (loading pump 4 ml/min MECN), affording 2-oxo-N-(1H-pyrazolo[4,3-c]pyridin-7-yl)-2-[(2R,5S)-5-methyl-2-[2-(1,2,2,6,6-pentamethyl-4-piperidyl) indazol-5-yl]-1-piperidyl]acetamide (18.6 mg, 29.54 μmol, 44.65% yield, 2HCl) ¹H NMR (600 MHz, dmso) δ 1.01-1.09 (m, 3H), 1.11-1.18 (m, 12H), 1.31-1.42 (m, 1H), 1.78-2.00 (m, 6H), 2.09-2.33 (m, 5H), 2.83-2.93 (m, 1H), 3.71-4.10 (m, 1H), 4.78-4.92 (m, 1H), 5.43-5.75 (m, 1H), 7.16-7.30 (m, 1H), 7.54-7.66 (m, 2H), 8.22 (s, 1H), 8.27-8.44 (m, 2H), 8.45-8.63 (m, 1H), 8.70-9.16 (m, 2H), 11.08-11.37 (m, 1H), 13.12-13.38 (m, 1H). LCMS(ESI): [M+H]⁺ m/z: calcd 556.4; found 557.6; Rt=1.828 min.

### Example 83. Synthesis of 2-oxo-N-(1H-pyrazolo[4,3-c]pyridin-7-yl)-2-[(2R,5S)-5-methyl-2-[2-[rel-(4R)-1,2,2-trimethyl-4-piperidyl]indazol-5-yl]-1-piperidyl]acetamide (Compound 20) and 2-oxo-N-(1H-pyrazolo[4,3-c]pyridin-7-yl)-2-[(2R,5S)-5-methyl-2-[2-[rel-(4S)-1,2,2-trimethyl-4-piperidyl]indazol-5-yl]-1-piperidyl]acetamide (Compound 75)

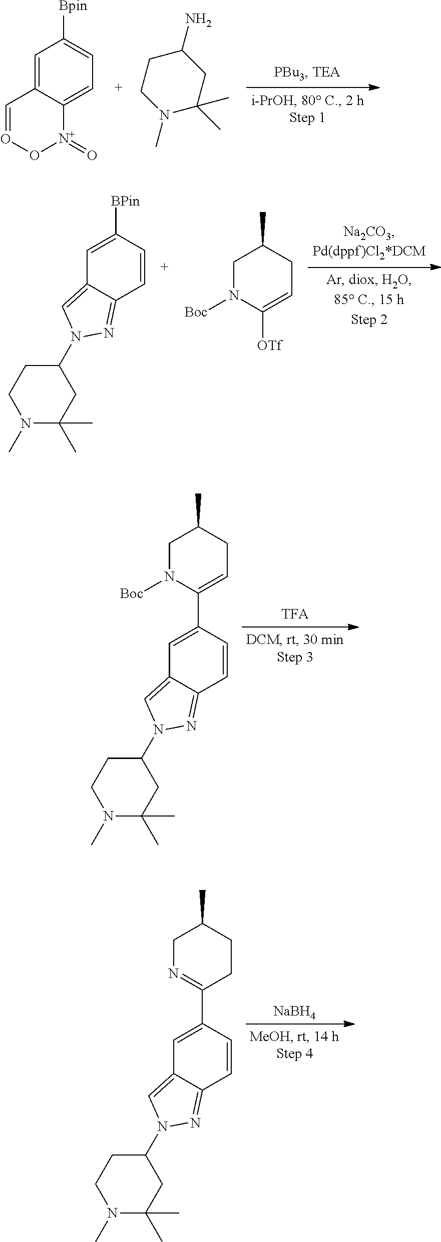

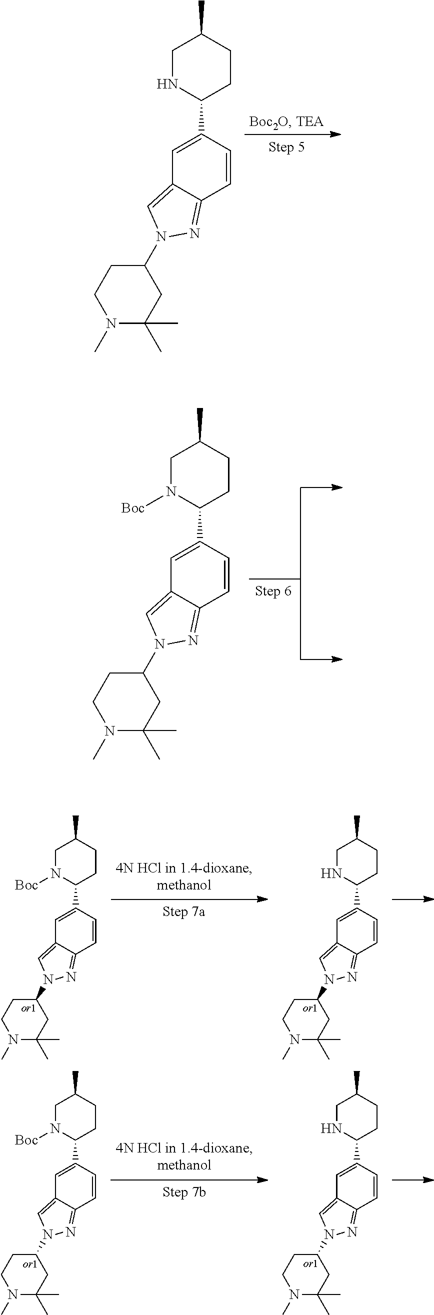

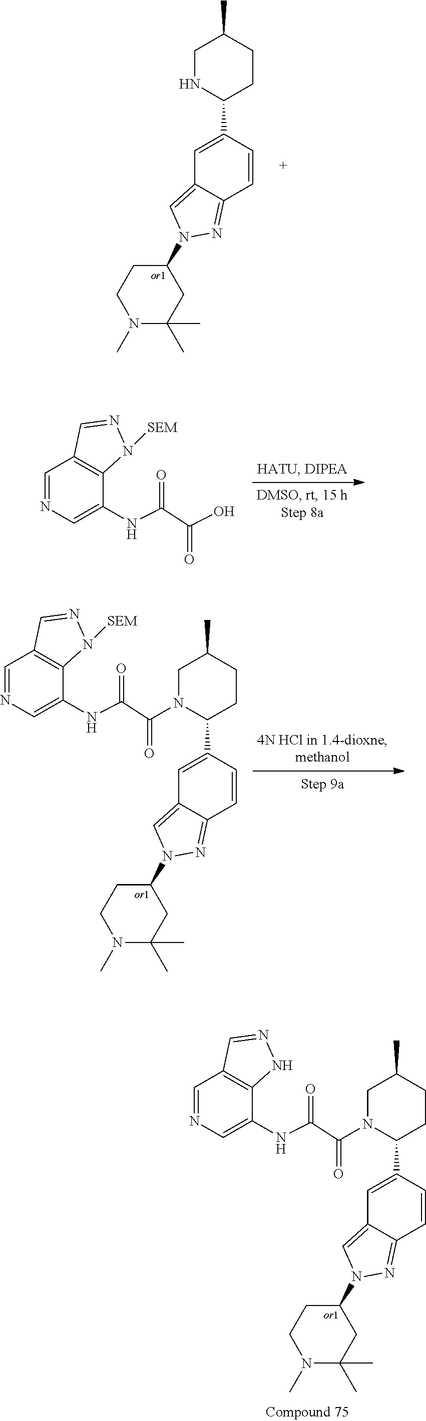 [SMILES: C[C@H]1CC[C@H](c2ccc3nn([C@@H]4CCN(C)C(C)(C)C4)cc3c2)NC1 ; C[Si](C)(C)CCOCn1ncc2cncc(NC(=O)C(=O)O)c21 ; C[C@H]1CC[C@H](c2ccc3nn([C@@H]4CCN(C)C(C)(C)C4)cc3c2)N(C(=O)C(=O)Nc2cncc3cnn(COCC[Si](C)(C)C)c23)C1 ; C[C@H]1CC[C@H](c2ccc3nn([C@@H]4CCN(C)C(C)(C)C4)cc3c2)N(C(=O)C(=O)Nc2cncc3cn[nH]c23)C1]

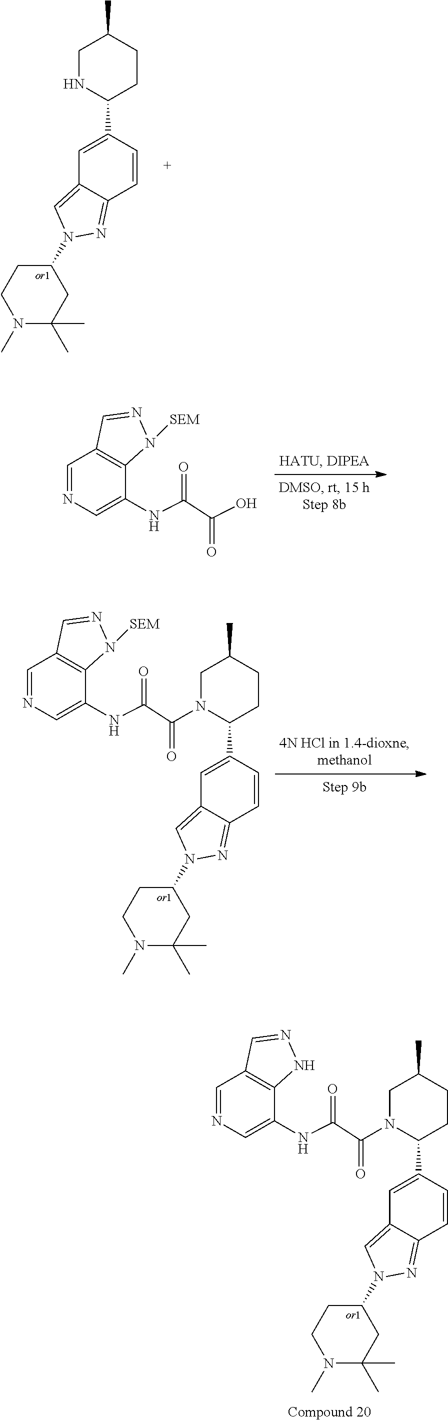 [SMILES: C[C@H]1CC[C@H](c2ccc3nn([C@H]4CCN(C)C(C)(C)C4)cc3c2)NC1 ; C[Si](C)(C)CCOCn1ncc2cncc(NC(=O)C(=O)O)c21 ; C[C@H]1CC[C@H](c2ccc3nn([C@H]4CCN(C)C(C)(C)C4)cc3c2)N(C(=O)C(=O)Nc2cncc3cnn(COCC[Si](C)(C)C)c23)C1 ; C[C@H]1CC[C@H](c2ccc3nn([C@H]4CCN(C)C(C)(C)C4)cc3c2)N(C(=O)C(=O)Nc2cncc3cn[nH]c23)C1]

### Step 1: The Synthesis of 5-(4,4,5,5-tetramethyl-1,3,2-dioxaborolan-2-yl)-2-(1,2,2-trimethyl-4-piperidyl)indazole

Prepared by general procedure Scheme S6 Step 1A (The crude product was purified by FCC (MeOH in MTBE from 0% to 100%).

Yield: 4.6 g (47.88%); LCMS(ESI): [M+H]⁺ m/z: calcd 369.2; found 370.2; Rt=1.089 min.

### Step 2: The Synthesis of tert-butyl-(3S)-3-methyl-6-[2-(1,2,2-trimethyl-4-piperidyl) indazol-5-yl]-3,4-dihydro-2H-pyridine-1-carboxylate

### Prepared by General Procedure Scheme S6 Step 2

The obtained product was used in the next step without further purification.

Yield: 7.3 g (crude); LCMS(ESI): [M+H]⁺ m/z: calcd 438.3; found 439.4; Rt=1.032 min.

### Step 3: The Synthesis of 5-[(3S)-3-methyl-2,3,4,5-tetrahydropyridin-6-yl]-2-(1,2,2-trimethyl-4-piperidyl)indazole

### Prepared by General Procedure Scheme S6 Step 3 (Method A)

The obtained product was used in the next step without further purification.

Yield: 7 g (crude); LCMS(ESI): [M+H]⁺ m/z: calcd 338.2; found 339.2; Rt=0.698 min.

### Step 4: The Synthesis of 5-[(2R,5S)-5-methyl-2-piperidyl]-2-(1,2,2-trimethyl-4-piperidyl)indazole

### Prepared by General Procedure Scheme S6 Step 4

Yield: 5.7 g (80.95%); LCMS(ESI): [M+H]⁺ m/z: calcd 340.2; found 341.2; Rt=0.716 min.

### Step 5: The Synthesis of tert-butyl (2R,5S)-5-methyl-2-[2-(1,2,2-trimethyl-4-piperidyl) indazol-5-yl]piperidine-1-carboxylate

Di-tert-butyl dicarbonate (3.65 g, 16.74 mmol, 3.84 mL) was added dropwise to the solution of 5-[-(2R,5S)-5-methyl-2-piperidyl]-2-(1,2,2-trimethyl-4-piperidyl)indazole (5.70 g, 16.74 mmol) and TEA (2.03 g, 20.09 mmol, 2.80 mL) in DCM (60 mL). After addition was complete, resulting mixture was stirred at 20° C. for 12 hr. The volatiles were removed under reduced pressure. The crude product was purified by FCC (methanol in mtbe from 0% to 100%). The reaction is successful. tert-butyl-(2R,5S)-5-methyl-2-[2-(1,2,2-trimethyl-4-piperidyl) indazol-5-yl]piperidine-1-carboxylate (0.75 g, 1.70 mmol, 10.17% yield) was obtained as a brown gum.

LCMS(ESI): [M+H]⁺ m/z: calcd 440.3; found 441.4; Rt=1.281 min.

### Step 6: The Synthesis of (2R,5S)-5-methyl-2-[2-[rel-(4S)-1,2,2-trimethyl-4-piperidyl]indazol-5-yl]piperidine-1-carboxylate and (2R,5S)-5-methyl-2-[2-[rel-(4R)-1,2,2-trimethyl-4-piperidyl]indazol-5-yl]piperidine-1-carboxylate

Chiral resolution was done in the following conditions:

Chiralpak AD-H III (250*20 mm, 5 mkm), Hexane-IPA-MeOH, 80-10-10, 12 ml/min to give: tert-butyl (2R,5S)-5-methyl-2-[2-[rel-(4S)-1,2,2-trimethyl-4-piperidyl]indazol-5-yl]piperidine-1-carboxylate (0.44 g, 998.59 μmol, 58.67% yield) as 1st isomer, with ret.time=17.464 min (analytical), 15.646 min (preparative). RT (Chiralpak AD-H (250*4.6 mm, 5 mkm), Hexane-IPA-MeOH, 80-10-10, 0.6 ml/min)=17.284 min.

LCMS(ESI): [M+H]⁺ m/z: calcd 440.3; found 441.4; Rt=1.269 min.

tert-butyl (2R,5S)-5-methyl-2-[2-[rel-(4R)-1,2,2-trimethyl-4-piperidyl]indazol-5-yl]piperidine-1-carboxylate (0.255 g, 578.73 μmol, 34.00% yield) as 2nd isomer, with ret. time=27.51 min (analytical), 26.586 min (preparative).

RT (Chiralpak AD-H (250*4.6 mm, 5 mkm), Hexane-IPA-MeOH, 80-10-10, 0.6 ml/min)=26.909 min.

LCMS(ESI): [M+H]⁺ m/z: calcd 440.3; found 441.4; Rt=1.269 min.

### Step 7: The Synthesis of 5-[(2R,5S)-5-methyl-2-piperidyl]-2-[rel-(4S)-1,2,2-trimethyl-4-piperidyl]indazole and 5-[(2R,5S)-5-methyl-2-piperidyl]-2-[rel-(4R)-1,2,2-trimethyl-4-piperidyl]indazole

tert-butyl (2R,5S)-5-methyl-2-[2-[rel-(4R or 4S)-1,2,2-trimethyl-4-piperidyl]indazol-5-yl]piperidine-1-carboxylate (0.255 g, 578.73 μmol) were dissolved in a mixture of MeOH (5 mL) and Dioxane (HCl) (5 mL). The resulting clear solution was stirred for 4 hr at 20° C. The reaction mixture was concentrated on vacuo. The reaction is successful. 5-[(2R,5S)-5-methyl-2-piperidyl]-2-[rel-(4R or 4S)-1,2,2-trimethyl-4-piperidyl]indazole (0.25 g, 555.70 μmol, 96.02% yield, ³HCl) were obtained as a light-yellow solids.

LCMS(ESI): [M+H]⁺ m/z: calcd 340.2; found 341.2; Rt=0.66 min for 5-[(2R,5S)-5-methyl-2-piperidyl]-2-[rel-(4S)-1,2,2-trimethyl-4-piperidyl]indazole from 1^{st} isomer.

LCMS(ESI): [M+H]⁺ m/z: calcd 340.2; found 341.2; Rt=0.65 min for 5-[(2R,5S)-5-methyl-2-piperidyl]-2-[rel-(4R)-1,2,2-trimethyl-4-piperidyl]indazole from 2^{nd} isomer.

### Step 8: The Synthesis of N-(6-amino-5-ethyl-3-pyridyl)-2-oxo-2-[(2R,5S)-5-methyl-2-[2-[rel-(4S)-1,2,2-trimethyl-4-piperidyl]indazol-5-yl]-1-piperidyl]acetamide and N-(6-amino-5-ethyl-3-pyridyl)-2-oxo-2-[(2R,5S)-5-methyl-2-[2-[rel-(4R)-1,2,2-trimethyl-4-piperidyl]indazol-5-yl]-1-piperidyl]acetamide

### Prepared by General Procedure Scheme S6 Step 5

HPLC conditions: 2-10 min 55-70% methanol+nh3 flow 30 ml/min (loading pump 4 ml/min methanol), Column Sun Fire C18 100*19 mm.

Yield: 0.041 g, (34.69%)

LCMS(ESI): [M+H]⁺ m/z: calcd 658.9; found 660.4; Rt=1.229 min. for N-(6-amino-5-ethyl-3-pyridyl)-2-oxo-2-[(2R,5S)-5-methyl-2-[2-[rel-(4S)-1,2,2-trimethyl-4-piperidyl]indazol-5-yl]-1-piperidyl]acetamide from 1^{st} isomer

Yield: 0.125 g, (56.90%); LCMS(ESI): [M+H]⁺ m/z: calcd 658.9; found 660.4; Rt=1.232 min. for N-(6-amino-5-ethyl-3-pyridyl)-2-oxo-2-[(2R,5S)-5-methyl-2-[2-[rel-(4R)-1,2,2-trimethyl-4-piperidyl]indazol-5-yl]-1-piperidyl]acetamide from 2^{nd} isomer

### Step 9a: The Synthesis of 2-oxo-N-(1H-pyrazolo[4,3-c]pyridin-7-yl)-2-[(2R,5S)-5-methyl-2-[2-[rel-(4S)-1,2,2-trimethyl-4-piperidyl]indazol-5-yl]-1-piperidyl]acetamide (Compound 75)

2-oxo-2-[-(2R,5S)-5-methyl-2-[2-[rel-(4S)-1,2,2-trimethyl-4-piperidyl]indazol-5-yl]-1-piperidyl]-N-[1-(2-trimethylsilylethoxymethyl)pyrazolo[4,3-c]pyridin-7-yl]acetamide (0.097 g, 147.21 μmol) was dissolved in TFA (503.57 mg, 4.42 mmol, 340.25 μL) and stirred at 20° C. for 12 hr. The reaction mixture was submitted to HPLC (2-10 min 40-60% methanol+nh3 flow 30 ml/min (loading pump 4 ml/min methanol), Column Sun Fire C18 100*19 mm). The reaction is successful. 2-oxo-N-(1H-pyrazolo[4,3-c]pyridin-7-yl)-2-[-(2R,5S)-5-methyl-2-[2-[rel-(4S)-1,2,2-trimethyl-4-piperidyl]indazol-5-yl]-1-piperidyl]acetamide (0.039 g, 73.77 μmol, 50.11% yield) was obtained as a light-yellow solid.

Obtained from 1^{st} Isomer

¹H NMR (DMSO-d6, 500 MHz): δ (ppm) 1.06 (m, 9H), 1.37 (m, 1H), 1.79 (m, 1H), 1.92 (m, 3H), 2.08 (m, 3H), 2.19 (s, 3H), 2.29 (m, 1H), 2.68 (m, 3H), 3.99 (m, 1H), 4.66 (m, 1H), 5.61 (m, 1H), 7.03 (m, 1H), 7.55 (s, 1H), 7.67 (m, 1H), 8.31 (m, 2H), 8.49 (m, 1H), 8.93 (m, 1H), 11.12 (m, 1H), 13.10 (m, 1H); LCMS(ESI): [M+H]⁺ m/z: calcd 528.6; found 530.2.

Rt=1.715 min.

### Step 9b: The Synthesis of 2-oxo-N-(1H-pyrazolo[4,3-c]pyridin-7-yl)-2-[(2R,5S)-5-methyl-2-[2-[rel-(4R)-1,2,2-trimethyl-4-piperidyl]indazol-5-yl]-1-piperidyl]acetamide (Compound 20)

2-oxo-2-[-(2R,5S)-5-methyl-2-[2-[rel-(4R)-1,2,2-trimethyl-4-piperidyl]indazol-5-yl]-1-piperidyl]-N-[1-(2-trimethylsilylethoxymethyl)pyrazolo[4,3-c]pyridin-7-yl]acetamide (0.125 g, 189.71 μmol) was dissolved in TFA (648.93 mg, 5.69 mmol, 438.47 μL) and stirred at 20° C. for 12 hr. The reaction mixture was submitted to HPLC (2-10 min 40-60% methanol+nh3 flow 30 ml/min (loading pump 4 ml/min methanol), Column Sun Fire C18 100*19 mm). The reaction is successful. 2-oxo-N-(1H-pyrazolo[4,3-c]pyridin-7-yl)-2-[-(2R,5S)-5-methyl-2-[2-[rel-(4R)-1,2,2-trimethyl-4-piperidyl]indazol-5-yl]-1-piperidyl]acetamide (0.053 g, 100.26 μmol, 52.85% yield) was obtained as a light-yellow solid.

Obtained from 2^{nd} Isomer

¹H NMR (DMSO-d6, 500 MHZ): δ (ppm) 1.05 (m, 9H), 1.37 (m, 1H), 1.77 (m, 1H), 1.91 (m, 3H), 2.07 (m, 3H), 2.18 (s, 3H), 2.29 (m, 1H), 2.71 (m, 4H), 3.93 (m, 1H), 4.66 (m, 1H), 5.61 (m, 1H), 7.03 (m, 1H), 7.55 (s, 1H), 7.67 (m, 1H), 8.34 (m, 2H), 8.49 (m, 1H), 8.93 (m, 1H), 11.12 (m, 1H), 13.10 (m, 1H); LCMS(ESI): [M+H]⁺ m/z: calcd 528.6; found 529.2.

Rt=1.707 min.

### Example 84. Synthesis of 2-((2R,5S)-5-methyl-2-(2-(1-methylazetidin-3-yl)-2H-indazol-5-yl)piperidin-1-yl)-2-oxo-N-(1H-pyrazolo[4,3-c]pyridin-7-yl)acetamide (Compound 66)

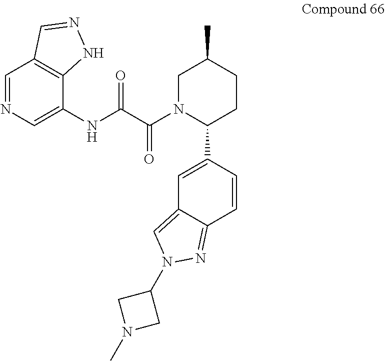 [SMILES: C[C@H]1CC[C@H](c2ccc3nn(C4CN(C)C4)cc3c2)N(C(=O)C(=O)Nc2cncc3cn[nH]c23)C1]

The compound was prepared in accordance with general Scheme S6 (including Step 1, step 2, step 3 method A, step 4, Step 5 using Intermediate 37)

Compound 66 ¹H NMR (600 MHz, dmso) δ 0.84-1.07 (m, 3H), 1.23-1.44 (m, 1H), 1.69-1.99 (m, 2H), 2.06-2.18 (m, 1H), 2.21-2.31 (m, 1H), 2.31-2.37 (m, 3H), 2.84-3.08 (m, 1H), 3.44-3.55 (m, 2.7H), 3.74-3.81 (m, 2H), 4.04-4.10 (m, 0.3H), 5.13-5.37 (m, 1H), 5.43-5.81 (m, 1H), 7.18-7.32 (m, 1H), 7.55-7.74 (m, 2H), 8.28-8.39 (m, 1H), 8.39-8.67 (m, 2H), 8.85-8.98 (m, 1H), 10.87-11.31 (m, 1H), 13.09 (br s, 1H).

LCMS(ESI): [M+H]⁺ m/z: calcd 472.5; found 473.2; Rt=1.420 min.

### Example 85. Synthesis of N-(8-amino-2,7-naphthyridin-4-yl)-2-oxo-2-[(2R,5S)-5-methyl-2-[2-(1-methyl-4-piperidyl) indazol-5-yl]-1-piperidyl]acetamide (Compound 51

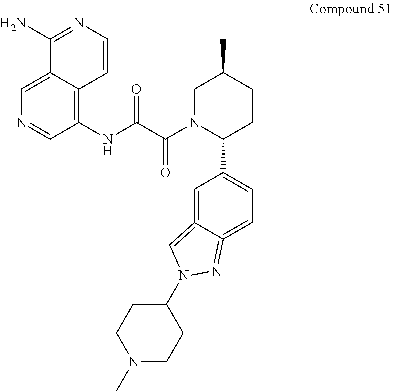

The compound was prepared in accordance with general Scheme S6 (including Step 1A, Step 1, Step 2, Step 3 Method B, Step 4), using Intermediate 42.

LCMS(ESI): [M+H]⁺ m/z: calcd 526.6; found 527.4; Rt=1.923 min.

### Example 86. Synthesis of N-imidazo[1,2-a]pyridin-7-yl-2-oxo-2-[(2R,5S)-5-methyl-2-[2-(1-methyl-4-piperidyl) indazol-5-yl]-1-piperidyl]acetamide (Compound 15)

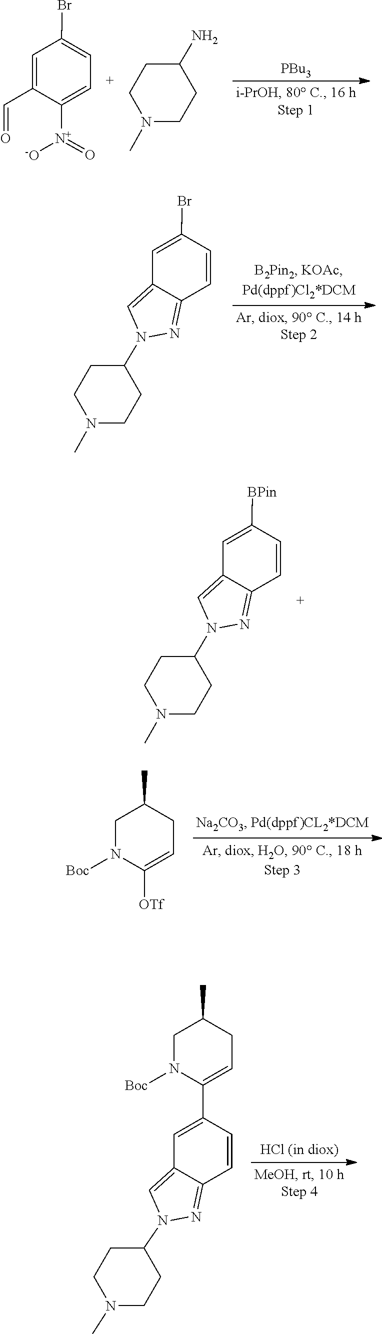

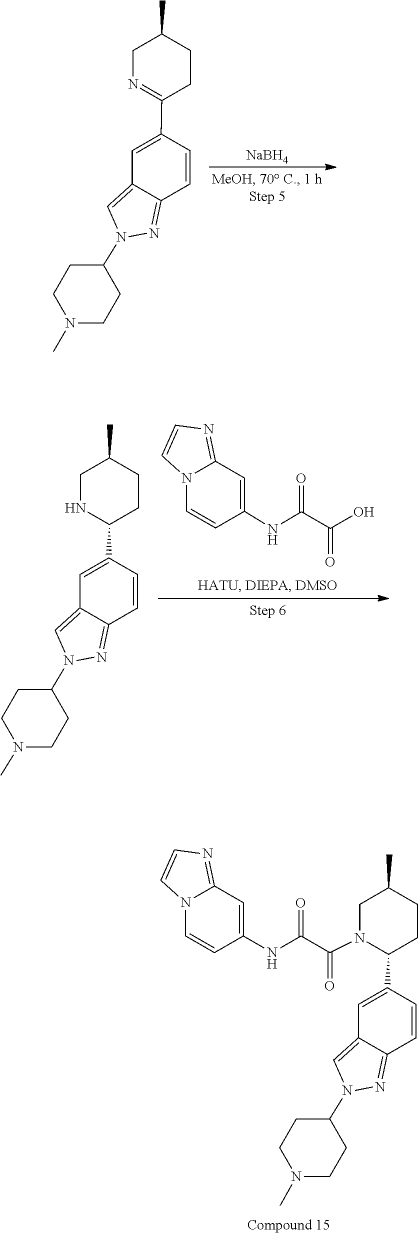

### Step 1: The Synthesis of 5-Bromo-2-(1-methyl-4-piperidyl)indazole

### Prepared by General Procedure Scheme S6 Step 1A

Yield: 20 g (78.19%); LCMS(ESI): [M+H]⁺ m/z: calcd 295.0; found 295.0; Rt=0.785 min.

### Step 2: The Synthesis of 2-(1-Methyl-4-piperidyl)-5-(4,4,5,5-tetramethyl-1,3,2-dioxaborolan-2-yl)indazole

### Prepared by General Procedure Scheme S6 Step 1

FCC conditions: Interchim; 330 g SiO₂, MTBE-MeOH from 0˜100%, flow rate=100 mL/min, cv=9.4

Yield: 9.0 g (77.59%); LCMS(ESI): [M+H]⁺ m/z: calcd 342.2; found 342.2; Rt=0.799 min.

### Step 3: The Synthesis of tert-Butyl (3S)-3-methyl-6-[2-(1-methyl-4-piperidyl) indazol-5-yl]-3,4-dihydro-2H-pyridine-1-carboxylate

### Prepared by General Procedure Scheme S6 Step 2

FCC conditions: Interchim; 120 g SiO₂, MTBE-MeOH from 0˜100%, flow rate=70 mL/min, cv=15.3

Yield: 6.0 g (55.41%); LCMS(ESI): [M+H]⁺ m/z: calcd 411.2; found 411.5; Rt=1.019 min.

### Step 4: The Synthesis of 2-(1-Methyl-4-piperidyl)-5-[(3S)-3-methyl-2,3,4,5-tetrahydropyridin-6-yl]indazole

### Prepared by General Procedure Scheme S6 Step 3 (Method B)

Yield: 1.2 g (79.35%); LCMS(ESI): [M+H]⁺ m/z: calcd 311.2; found 311.2; Rt=0.769 min.

### Step 5: The Synthesis of 2-(1-Methyl-4-piperidyl)-5-[(2R,5S)-5-methyl-2-piperidyl]indazole

### Prepared by General Procedure Scheme S6 Step 4

Yield: 3.2 g (79.48%); LCMS(ESI): [M+H]⁺ m/z: calcd 313.2; found 313.2; Rt=0.664 min.

### Step 6: The Synthesis of N-imidazo[1,2-a]pyridin-7-yl-2-oxo-2-[(2R,5S)-5-methyl-2-[2-(1-methyl-4-piperidyl) indazol-5-yl]-1-piperidyl]acetamide (Compound 15)

2-(1-methyl-4-piperidyl)-5-[(2R,5S)-5-methyl-2-piperidyl]indazole (220.41 mg, 409.32 μmol, 2TFA), 2-(imidazo[1,2-a]pyridin-7-ylamino)-2-oxo-acetic acid (83.98 mg, 409.32 μmol), HATU (171.20 mg, 450.25 μmol) and DIPEA (211.60 mg, 1.64 mmol, 285.18 μL) were dissolved in DMSO (2 mL) and stirred at 25° C. for 12 hr. The solution in DMSO was subjected to HPLC ((2-10 min 40-100% methanol+nh3 flow 30 ml/min (loading pump 4 ml/min, methanol), Column Sun Fire C18 100*19 mm). After purification was obtained N-imidazo[1,2-a]pyridin-7-yl-2-oxo-2-[(2R,5S)-5-methyl-2-[2-(1-methyl-4-piperidyl) indazol-5-yl]-1-piperidyl]acetamide (0.047 g, 94.07 μmol, 22.98% yield)

¹H NMR (600 MHz, dmso) § 0.99-1.05 (m, 3H), 1.29-1.42 (m, 1H), 1.73-1.94 (m, 2H), 1.99-2.15 (m, 7H), 2.20-2.31 (m, 4H), 2.85-2.90 (m, 2H), 3.40-4.47 (m, 3H), 5.17-5.71 (m, 1H), 6.97-7.11 (m, 1H), 7.14-7.28 (m, 1H), 7.42-7.51 (m, 1H), 7.56-7.67 (m, 2H), 7.78-7.86 (m, 1H), 7.87-8.03 (m, 1H), 8.33-8.50 (m, 2H), 11.05 (s, 1H).

LCMS(ESI): [M+H]⁺ m/z: calcd 499.6; found 500.2; Rt=1.359 min.

### Example 87. Synthesis of 2-oxo-N-(1H-pyrazolo[4,3-c]pyridin-7-yl)-2-[(2R,5S)-5-methyl-2-[2-[(3R)-1-methylpyrrolidin-3-yl]indazol-5-yl]-1-piperidyl]acetamide (Compound 36)

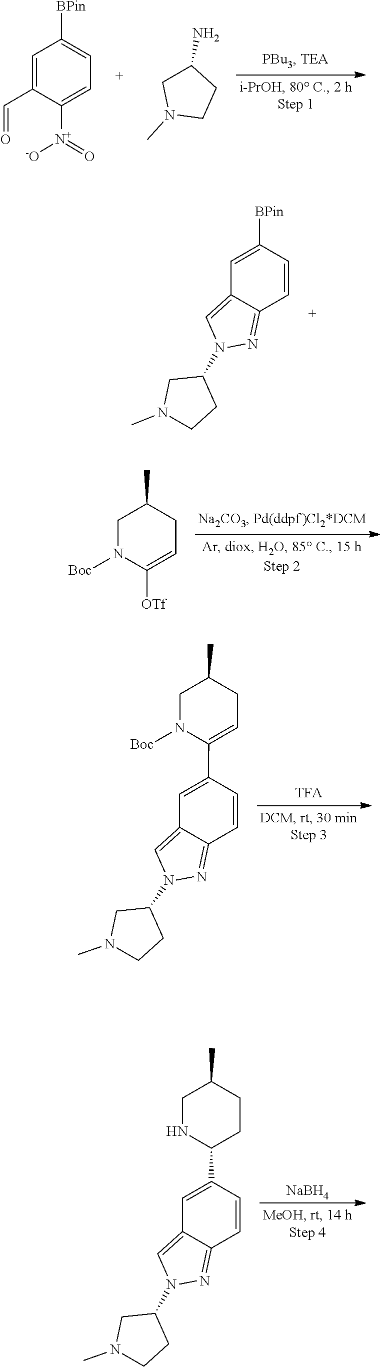

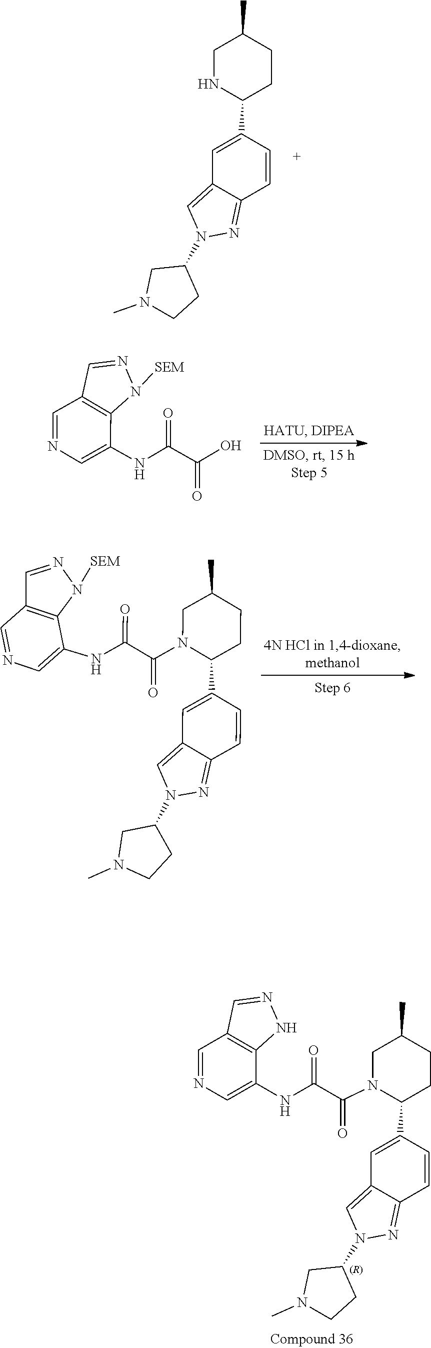

### Step 1: The Synthesis of 2-[(3R)-1-Methylpyrrolidin-3-yl]-5-(4,4,5,5-tetramethyl-1,3,2-dioxaborolan-2-yl)indazole

### Prepared by General Procedure Scheme S6 Step 1

FCC conditions: Interchim; 120 g SiO₂, MTBE-MeOH from 0˜100%, flow rate=70 mL/min, cv=13.7

Yield: 3.6 g (43.55%); LCMS(ESI): [M+H]⁺ m/z: calcd 328.2; found 328.2; Rt=0.897 min.

### Step 2: The Synthesis of tert-Butyl (3S)-3-methyl-6-[2-[(3R)-1-methylpyrrolidin-3-yl]indazol-5-yl]-3,4-dihydro-2H-pyridine-1-carboxylate

### Prepared by General Procedure Scheme S6 Step 2.

Yield: 3.5 g (crude); LCMS(ESI): [M+H]⁺ m/z: calcd 397.2; found 397.4; Rt=1.010 min.

### Step 3: The Synthesis of 2-[(3R)-1-Methylpyrrolidin-3-yl]-5-[(3S)-3-methyl-2,3,4,5-tetrahydropyridin-6-yl]indazole

### Prepared by General Procedure Scheme S6 Step 3 (Method A)

Yield: 2.0 g (crude); LCMS(ESI): [M+H]⁺ m/z: calcd 297.2; found 297.1; Rt=0.380 min.

### Step 4: The Synthesis of 5-[(2R,5S)-5-Methyl-2-piperidyl]-2-[(3R)-1-methylpyrrolidin-3-yl]indazole

### Prepared by General Procedure Scheme S6 Step 4

Yield: 1.9 g (crude, 2 HCl); LCMS(ESI): [M+H]⁺ m/z: calcd 299.2; found 299.0; Rt=0.675 min.

### Step 5: The Synthesis of 2-oxo-2-[(2R,5S)-5-methyl-2-[2-[(3R)-1-methylpyrrolidin-3-yl]indazol-5-yl]-1-piperidyl]-N-[1-(2-trimethylsilylethoxymethyl)pyrazolo[4,3-c]pyridin-7-yl]acetamide

### Prepared by General Procedure Scheme S6 Step 5 Using Intermediate 37

HPLC conditions: 2-10 min 25-50% water ACN+FA 30/min; loading pump 4 ml/min ACN column, SunFire 19*100 mm.

Yield: 0.107 g, (21.47%); LCMS(ESI): [M+H]⁺ m/z: calcd 616.8; found 617.8; Rt=1.276 min.

### Step 6: The Synthesis of 2-oxo-N-(1H-pyrazolo[4,3-c]pyridin-7-yl)-2-[(2R,5S)-5-methyl-2-[2-[(3R)-1-methylpyrrolidin-3-yl]indazol-5-yl]-1-piperidyl]acetamide (Compound 36)

2-oxo-2-[(2R,5S)-5-methyl-2-[2-[(3R)-1-methylpyrrolidin-3-yl]indazol-5-yl]-1-piperidyl]-N-[1-(2-trimethylsilylethoxymethyl)pyrazolo[4,3-c]pyridin-7-yl]acetamide (0.107 g, 173.47 μmol) was stirred in a mixture of HCl-dioxane solution (4 mL) and methanol (0.5 mL) for 0.4 hr. Upon completion, the reaction mixture was concentrated under reduced pressure and the residue was submitted to HPLC (2-10 min 30-55% methanol+NH₃ flow 30 ml/min (loading pump 4 ml methanol), column: sun fire C18) to afford 2-oxo-N-(1H-pyrazolo[4,3-c]pyridin-7-yl)-2-[(2R,5S)-5-methyl-2-[2-[(3R)-1-methylpyrrolidin-3-yl]indazol-5-yl]-1-piperidyl]acetamide (0.014 g, 28.77 μmol, 16.59% yield).

¹H NMR (DMSO-d6, 600 MHZ): δ (ppm) 1.05 (m, 3H), 1.36 (m, 1H), 1.84 (m, 2H), 2.03 (m, 1H), 2.25 (m, 2H), 2.33 (s, 3H), 2.57 (m, 1H), 2.91 (m, 4H), 3.97 (m, 1H), 5.20 (m, 1H), 5.60 (m, 1H), 7.23 (m, 1H), 7.63 (m, 2H), 8.26 (m, 2H), 8.47 (m, 2H), 8.93 (m, 1H), 11.07 (m, 1H), 13.03 (m, 1H). LCMS(ESI): [M+H]⁺ m/z: calcd 486.6; found 487.2; Rt=2.046 min.

### Example 88. Synthesis of 2-oxo-N-(1H-pyrazolo[4,3-c]pyridin-7-yl)-2-[(2R,5S)-5-methyl-2-[2-[(3S)-1-methylpyrrolidin-3-yl]indazol-5-yl]-1-piperidyl]acetamide (Compound 104)

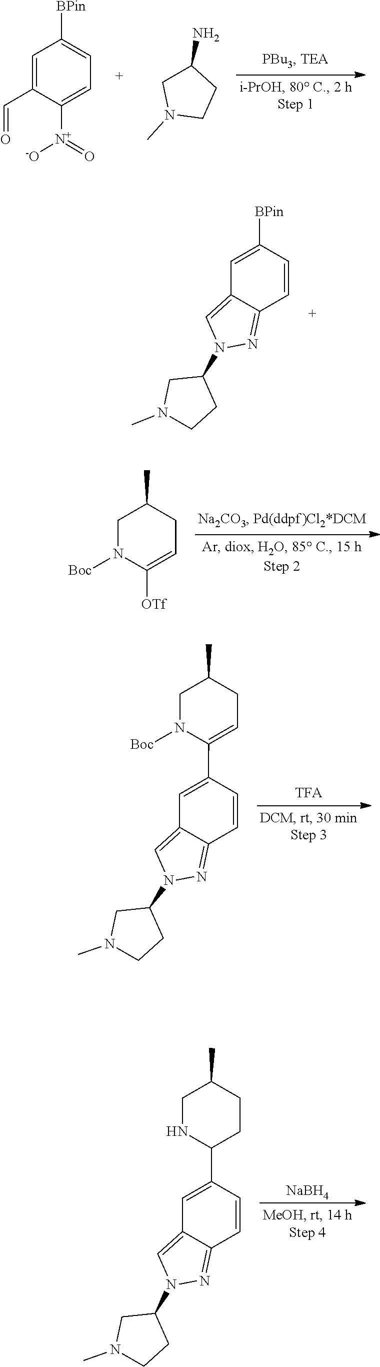

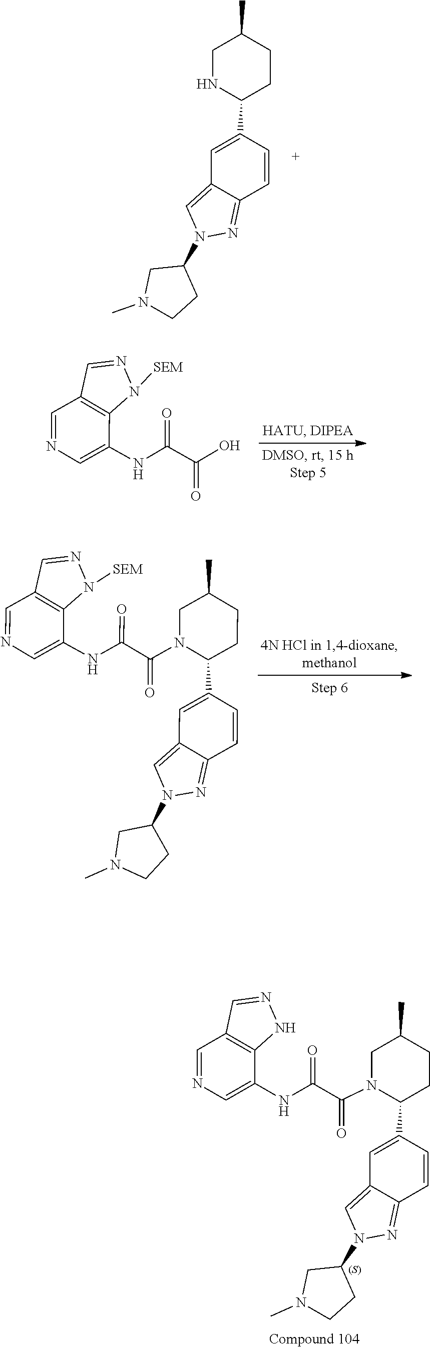

### Step 1: The Synthesis of 2-[(3S)-1-Methylpyrrolidin-3-yl]-5-(4,4,5,5-tetramethyl-1,3,2-dioxaborolan-2-yl)indazole

### Prepared by General Procedure Scheme S6 Step 1

FCC conditions: Interchim; 120 g SiO₂, MTBE-MeOH from 0˜100%, flow rate=70 mL/min, cv=13.9

Yield: 2.6 g (31.45%); LCMS(ESI): [M+H]⁺ m/z: calcd 328.2; found 328.2; Rt=0.904 min.

### Step 2: The Synthesis of tert-Butyl (3S)-3-methyl-6-[2-[(3S)-1-methylpyrrolidin-3-yl]indazol-5-yl]-3,4-dihydro-2H-pyridine-1-carboxylate

### Prepared by General Procedure Scheme S6 Step 2

Yield: 2.0 g (crude); LCMS(ESI): [M+H]⁺ m/z: calcd 397.2; found 397.2; Rt=1.189 min.

### Step 3: The Synthesis of 2-[(3S)-1-Methylpyrrolidin-3-yl]-5-[(3S)-3-methyl-2,3,4,5-tetrahydropyridin-6-yl]indazole

### Prepared by General Procedure Scheme S6 Step 3 (Method A)

Yield: 1.55 g (crude); LCMS(ESI): [M+H]⁺ m/z: calcd 297.2; found 297.2; Rt=0.668 min.

### Step 4: The Synthesis of 5-[(2R,5S)-5-Methyl-2-piperidyl]-2-[(3S)-1-methylpyrrolidin-3-yl]indazole

### Prepared by General Procedure Scheme S6 Step 4

Yield: 1.5 g (crude, 2HCl); LCMS(ESI): [M+H]⁺ m/z: calcd 299.2; found 299.2; Rt=0.680 min.

### Step 5: The Synthesis of 2-oxo-2-[(2R,5S)-5-methyl-2-[2-[(3R)-1-methylpyrrolidin-3-yl]indazol-5-yl]-1-piperidyl]-N-[1-(2-trimethylsilylethoxymethyl)pyrazolo[4,3-c]pyridin-7-yl]acetamide

### Prepared by General Procedure Scheme S6 Step 5 Using Intermediate 37.

HPLC conditions: 2-10 min 25-50% water ACN+FA 30/min; loading pump 4 ml/min ACN column, SunFire 19*100 mm.

Yield: 0.07 g, (14.05%); LCMS(ESI): [M+H]⁺ m/z: calcd 616.8; found 617.8; Rt=1.276 min.

### Step 6: The Synthesis of 2-oxo-N-(1H-pyrazolo[4,3-c]pyridin-7-yl)-2-[(2R,5S)-5-methyl-2-[2-[(3S)-1-methylpyrrolidin-3-yl]indazol-5-yl]-1-piperidyl]acetamide (Compound 104)

2-oxo-2-[(2R,5S)-5-methyl-2-[2-[(3S)-1-methylpyrrolidin-3-yl]indazol-5-yl]-1-piperidyl]-N-[1-(2-trimethylsilylethoxymethyl)pyrazolo[4,3-c]pyridin-7-yl]acetamide (0.07 g, 113.48 μmol) was stirred in a mixture of HCl-dioxane solution (4 mL) and methanol (0.5 mL) for 0.4 hr. Upon completion, the reaction mixture was concentrated under reduced pressure and the residue was submitted to HPLC (2-10 min 30-55% methanol+NH₃ flow 30 ml/min (loading pump 4 ml methanol), column: sun fire C18) to afford 2-oxo-N-(1H-pyrazolo[4,3-c]pyridin-7-yl)-2-[(2R,5S)-5-methyl-2-[2-[(3S)-1-methylpyrrolidin-3-yl]indazol-5-yl]-1-piperidyl]acetamide (0.012 g, 24.66 μmol, 21.73% yield).

¹H NMR (600 MHz, dmso) § 1.01-1.12 (m, 3H), 1.31-1.44 (m, 1H), 1.78-1.97 (m, 2H), 2.04-2.30 (m, 3H), 2.32-2.37 (m, 3H), 2.54-2.59 (m, 2H), 2.81-3.09 (m, 4H), 3.76-4.10 (m, 1H), 5.09-5.30 (m, 1H), 5.44-5.81 (m, 1H), 7.16-7.32 (m, 1H), 7.53-7.71 (m, 2H), 8.26-8.61 (m, 3H), 8.85-9.05 (m, 1H), 10.92-11.19 (m, 1H), 12.86-13.12 (m, 1H).

LCMS(ESI): [M+H]⁺ m/z: calcd 486.6; found 488.2; Rt=0.865 min.

### Example 89. Synthesis of 2-oxo-N-(1H-pyrazolo[4,3-c]pyridin-7-yl)-2-[(2R,5S)-5-methyl-2-[2-(1-methyl-4-piperidyl) indazol-6-yl]-1-piperidyl]acetamide (Compound 90)

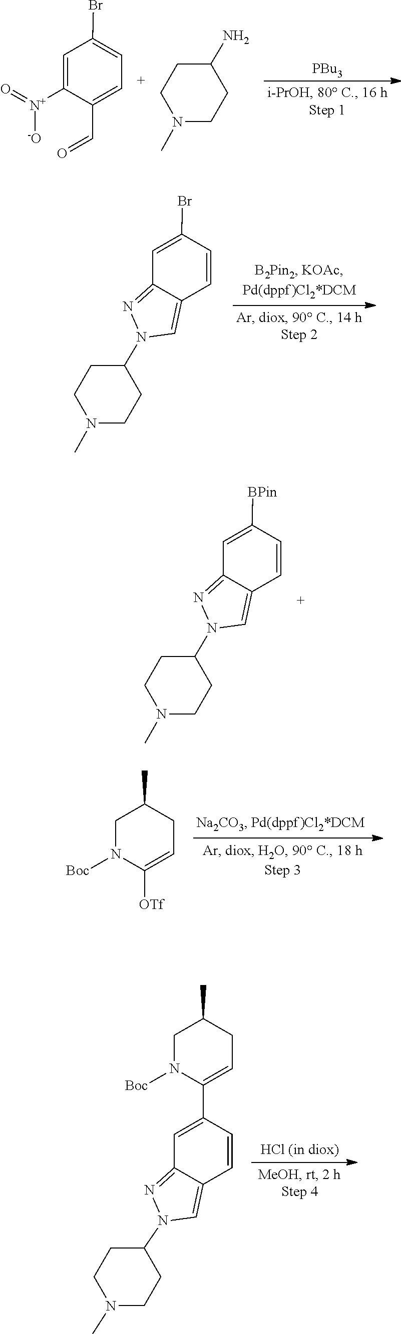

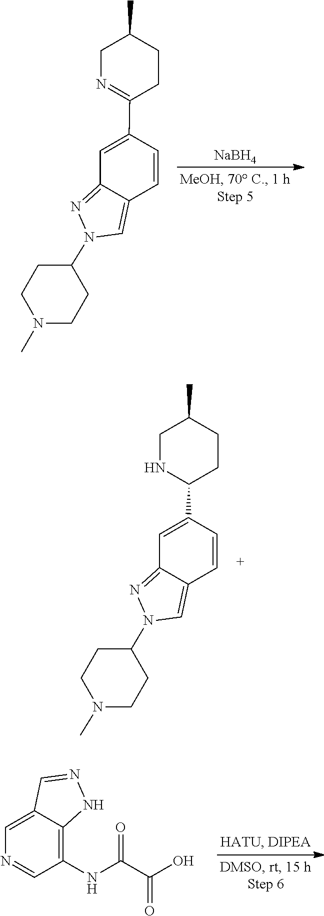

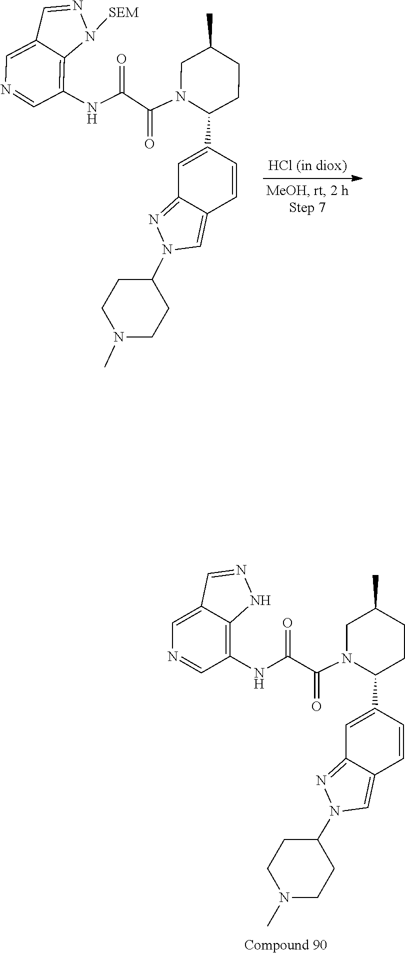 [SMILES: C[C@H]1CC[C@H](c2ccc3cn(C4CCN(C)CC4)nc3c2)N(C(=O)C(=O)Nc2cncc3cnn(COCC[Si](C)(C)C)c23)C1 ; C[C@H]1CC[C@H](c2ccc3cn(C4CCN(C)CC4)nc3c2)N(C(=O)C(=O)Nc2cncc3cn[nH]c23)C1]

### Step 1: The Synthesis of 6-Bromo-2-(1-methyl-4-piperidyl)indazole

### Prepared by General Procedure Scheme S7 Step 1A

FCC conditions: Interchim; 330 g SiO₂, MTBE-MeOH from 0˜100%, flow rate=100 mL/min, cv=10

Yield: 38.0 g (74.28%); LCMS(ESI): [M+2H]⁺ m/z: calcd 296.2; found 296.2; Rt=0.808 min.

### Step 2: The Synthesis of 2-(1-Methyl-4-piperidyl)-6-(4,4,5,5-tetramethyl-1,3,2-dioxaborolan-2-yl)indazole

### Prepared by General Procedure Scheme S7 Step 2A

FCC conditions: Interchim; 120 g SiO₂, MTBE-MeOH from 0˜100%, flow rate=70 mL/min, cv=7

Yield: 21.0 g (47.64%); LCMS(ESI): [M+H]⁺ m/z: calcd 342.2; found 342.0; Rt=1.045 min.

### Step 3: The Synthesis of tert-Butyl (3S)-3-methyl-6-[2-(1-methyl-4-piperidyl) indazol-6-yl]-3,4-dihydro-2H-pyridine-1-carboxylate

### Prepared by General Procedure Scheme S7 Step 2

FCC conditions: Interchim; 120 g SiO₂, MTBE-MeOH from 0˜100%, flow rate=70 mL/min, cv=11

Yield: 9.0 g (74.81%); LCMS(ESI): [M+H]⁺ m/z: calcd 411.2; found 411.4; Rt=1.165 min.

### Step 4: The Synthesis of 2-(1-Methyl-4-piperidyl)-6-[(3S)-3-methyl-2,3,4,5-tetrahydropyridin-6-yl]indazole

### Prepared by General Procedure Scheme S7 Step 3 (Method B)

Yield: 6.0 g (71.40%, 2HCl); LCMS(ESI): [M+H]⁺ m/z: calcd 311.2; found 311.2; Rt=0.578 min.

### Step 5: The Synthesis of 2-(1-Methyl-4-piperidyl)-6-[(2R,5S)-5-methyl-2-piperidyl]indazole

### Prepared by General Procedure Scheme S7 Step 4

Yield: 4.9 g (crude); LCMS(ESI): [M+H]⁺ m/z: calcd 313.2; found 313.2; Rt=0.543 min.

### Step 6: Synthesis of 2-oxo-2-[(2R,5S)-5-methyl-2-[2-(1-methyl-4-piperidyl) indazol-6-yl]-1-piperidyl]-N-[1-(2-trimethylsilylethoxymethyl)pyrazolo[4,3-c]pyridin-7-yl]acetamide

### Prepared by General Procedure Scheme S7 Step 5

HPLC conditions: Device (Mobile Phase, Column): SYSTEM 2-10 min 50-100% methanol flow 30 ml/min (loading pump 4 ml methanol), column: sun fire C18

Yield: 0.35 g (59.75%); LCMS(ESI): [M+H]⁺ m/z: calcd 630.4; found 631.4; Rt=1.211 min.

### Step 7: Synthesis of 2-oxo-N-(1H-pyrazolo[4,3-c]pyridin-7-yl)-2-[(2R,5S)-5-methyl-2-[2-(1-methyl-4-piperidyl) indazol-6-yl]-1-piperidyl]acetamide (Compound 90)

2-oxo-2-[(2R,5S)-5-methyl-2-[2-(1-methyl-4-piperidyl) indazol-6-yl]-1-piperidyl]-N-[1-(2-trimethylsilylethoxymethyl)pyrazolo[4,3-c]pyridin-7-yl]acetamide (0.35 g, 554.80 μmol) was dissolved in MeOH (3 mL) and HCl in Dioxan (554.80 μmol, 3 mL) was added thereto. Then it was stirred 25° C. for 1 hr. The reaction mixture was evaporated to afford crude product. It was purified by HPLC to give 2-oxo-N-(1H-pyrazolo[4,3-c]pyridin-7-yl)-2-[(2R,5S)-5-methyl-2-[2-(1-methyl-4-piperidyl) indazol-6-yl]-1-piperidyl]acetamide (0.11 g, 219.74 μmol, 39.61% yield).

Chromatography data: Device (Mobile Phase, Column): SYSTEM 2-10 min 30-60% ACN+NH3 30 ml/min (loading pump 4 ml ACN) column: SunFire 100*19 mm, 5 microM.

¹H NMR (600 MHz, dmso) δ 0.97-1.14 (m, 3H), 1.28-1.45 (m, 1H), 1.71-1.84 (m, 1H), 1.86-1.98 (m, 1H), 2.00-2.18 (m, 7H), 2.21 (s, 3H), 2.26-2.35 (m, 1H), 2.83-3.16 (m, 3H), 3.73-4.14 (m, 1H), 4.34-4.47 (m, 1H), 5.47-5.74 (m, 1H), 6.90-7.12 (m, 1H), 7.56 (s, 1H), 7.62-7.75 (m, 1H), 8.27-8.42 (m, 2H), 8.42-8.61 (m, 1H), 8.85-8.99 (m, 1H), 10.68-11.63 (m, 1H), 12.41-13.68 (m, 1H). LCMS(ESI): [M+H]⁺ m/z: calcd 500.3; found 501.4; Rt=1.865 min.

### Example 90. Synthesis of N-imidazo[1,2-a]pyridin-7-yl-2-oxo-2-[(2R,5S)-5-methyl-2-[2-(1-methyl-4-piperidyl) indazol-6-yl]-1-piperidyl]acetamide (Compound 88)

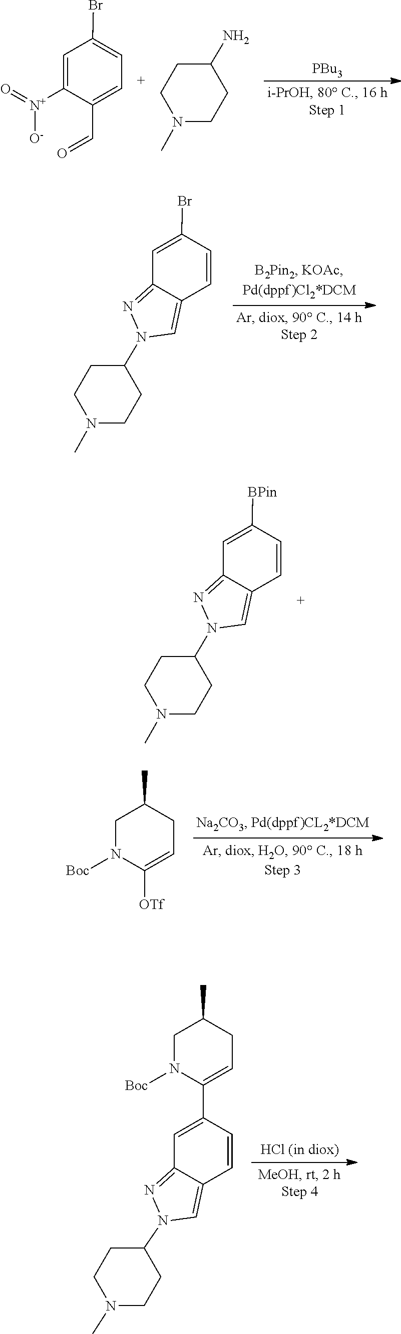

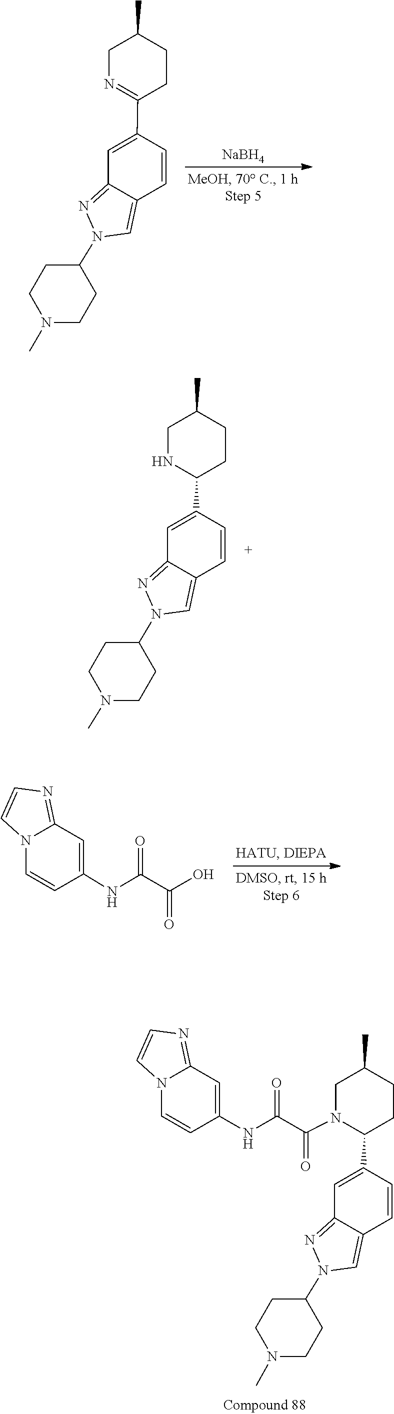 [SMILES: C[C@H]1CCC(c2ccc3cn(C4CCN(C)CC4)nc3c2)=NC1 ; "NaBH4" ; C[C@H]1CC[C@H](c2ccc3cn(C4CCN(C)CC4)nc3c2)NC1 ; O=C(O)C(=O)Nc1ccn2ccnc2c1 ; C[C@H]1CC[C@H](c2ccc3cn(C4CCN(C)CC4)nc3c2)N(C(=O)C(=O)Nc2ccn3ccnc3c2)C1]

### Step 1: The Synthesis of 6-Bromo-2-(1-methyl-4-piperidyl)indazole

### Prepared by General Procedure Scheme S7 Step 1A

FCC conditions: Interchim; 330 g SiO₂, MTBE-MeOH from 0˜100%, flow rate=100 mL/min, cv=10

Yield: 38.0 g (74.28%); LCMS(ESI): [M+2H]⁺ m/z: calcd 296.2; found 296.2; Rt=0.808 min.

### Step 2: The Synthesis of 2-(1-Methyl-4-piperidyl)-6-(4,4,5,5-tetramethyl-1,3,2-dioxaborolan-2-yl)indazole)

### Prepared by General Procedure Scheme S7 Step 2A

FCC conditions: Interchim; 120 g SiO₂, MTBE-MeOH from 0˜100%, flow rate=70 mL/min, cv=7

Yield: 21.0 g (47.64%); LCMS(ESI): [M+H]⁺ m/z: calcd 342.2; found 342.0; Rt=1.045 min.

### Step 3: The Synthesis of tert-Butyl (3S)-3-methyl-6-[2-(1-methyl-4-piperidyl) indazol-6-yl]-3,4-dihydro-2H-pyridine-1-carboxylate

### Prepared by General Procedure Scheme S7 Step 2

FCC conditions: Interchim; 120 g SiO₂, MTBE-MeOH from 0˜100%, flow rate=70 mL/min, cv=11

Yield: 9.0 g (74.81%); LCMS(ESI): [M+H]⁺ m/z: calcd 411.2; found 411.4; Rt=1.165 min.

### Step 4: The Synthesis of 2-(1-Methyl-4-piperidyl)-6-[(3S)-3-methyl-2,3,4,5-tetrahydropyridin-6-yl]indazole

### Prepared by General Procedure Scheme S7 Step 3 (Method B)

Yield: 6.0 g (71.40%, 2HCl); LCMS(ESI): [M+H]⁺ m/z: calcd 311.2; found 311.2; Rt=0.578 min.

### Step 5: The Synthesis of 2-(1-Methyl-4-piperidyl)-6-[(2R,5S)-5-methyl-2-piperidyl]indazole

### Prepared by General Procedure Scheme S7 Step 4

Yield: 4.9 g (crude); LCMS(ESI): [M+H]⁺ m/z: calcd 313.2; found 313.2; Rt=0.543 min.

### Step 6: Synthesis of N-imidazo[1,2-a]pyridin-7-yl-2-oxo-2-[(2R,5S)-5-methyl-2-[2-(1-methyl-4-piperidyl) indazol-6-yl]-1-piperidyl]acetamide (Compound 88)

### Prepared by General Procedure Scheme S7 Step 5

HPLC conditions: 2-10 min 40-50% R1+NH3 30 ml/min (loading pump 4 ml R¹) column: SunFire 100*19 mm, 5 microM

Yield: 0.13 g (16.26%)

¹H NMR (600 MHz, dmso) δ 0.98-1.11 (m, 3H), 1.29-1.42 (m, 1H), 1.71-1.82 (m, 1H), 1.84-1.95 (m, 1H), 2.01-2.17 (m, 7H), 2.21 (s, 3H), 2.25-2.34 (m, 1H), 2.82-3.19 (m, 3H), 3.47-4.08 (m, 1H), 4.36-4.47 (m, 1H), 5.18-5.72 (m, 1H), 6.93-7.04 (m, 1H), 7.05-7.12 (m, 1H), 7.41-7.51 (m, 1H), 7.51-7.58 (m, 1H), 7.64-7.73 (m, 1H), 7.76-7.86 (m, 1H), 7.86-8.05 (m, 1H), 8.33-8.39 (m, 1H), 8.39-8.50 (m, 1H), 10.98 (br s, 1H).

LCMS(ESI): [M+H]⁺ m/z: calcd 499.3; found 500.2; Rt=1.951 min.

### Example 91. Synthesis of N-(6-Amino-5-ethyl-3-pyridyl)-2-oxo-2-[(2R,5S)-5-methyl-2-[2-(1,2,2,6,6-pentamethyl-4-piperidyl) indazol-6-yl]-1-piperidyl]acetamide (Compound 45)

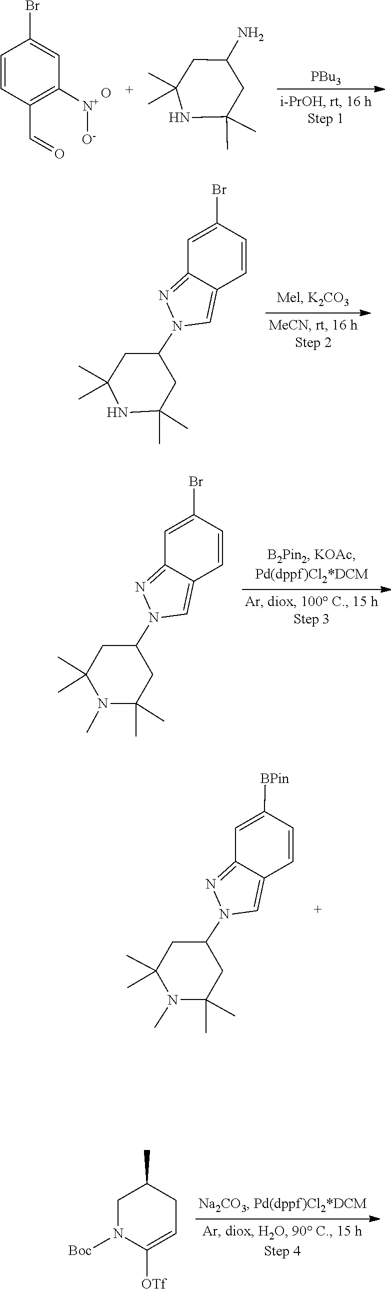

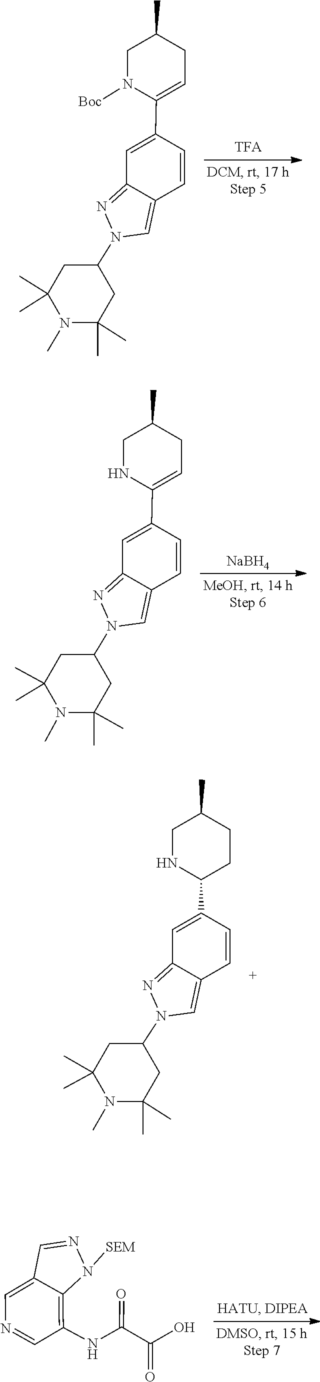 [SMILES: C[C@H]1CC=C(c2ccc3cn(C4CC(C)(C)N(C)C(C)(C)C4)nc3c2)N(C(=O)OC(C)(C)C)C1 ; C[C@H]1CC=C(c2ccc3cn(C4CC(C)(C)N(C)C(C)(C)C4)nc3c2)NC1 ; "NaBH4" ; C[C@H]1CC[C@H](c2ccc3cn(C4CC(C)(C)N(C)C(C)(C)C4)nc3c2)NC1 ; C[Si](C)(C)CCOCn1ncc2cncc(NC(=O)C(=O)O)c21]

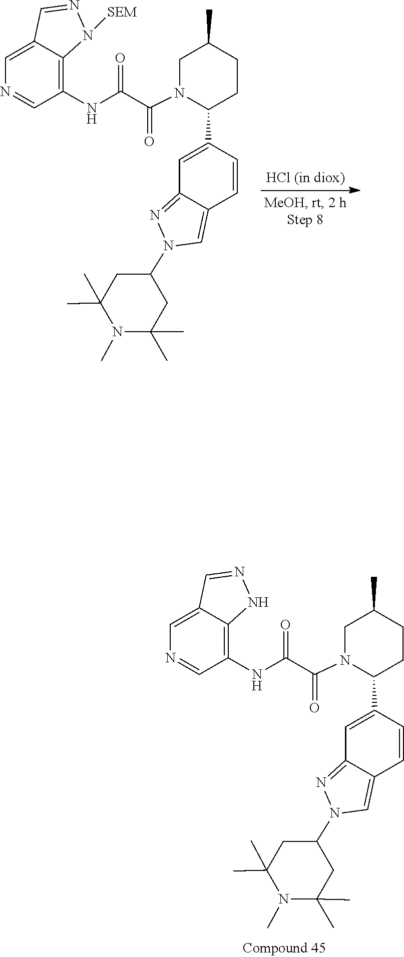 [SMILES: C[C@H]1CC[C@H](c2ccc3cn(C4CC(C)(C)N(C)C(C)(C)C4)nc3c2)N(C(=O)C(=O)Nc2cncc3cnn(COCC[Si](C)(C)C)c23)C1 ; C[C@H]1CC[C@H](c2ccc3cn(C4CC(C)(C)N(C)C(C)(C)C4)nc3c2)N(C(=O)C(=O)Nc2cncc3cn[nH]c23)C1]

### Step 1: The Synthesis of 6-Bromo-2-(2,2,6,6-tetramethyl-4-piperidyl)indazole

### Prepared by General Procedure Scheme S7 Step 1A

Yield: 3.6 g (82.08%); LCMS(ESI): [M+H]⁺ m/z: calcd 336.2; found 336.2; Rt=1.061 min.

### Step 2: The Synthesis of 6-Bromo-2-(1,2,2,6,6-pentamethyl-4-piperidyl)indazole

### Prepared by General Procedure Scheme S7 Step 1C

Yield: 2.7 g (72.0%); LCMS(ESI): [M+H]⁺ m/z: calcd 352.2; found 350.2; Rt=1.015 min.

### Step 3: The Synthesis of 2-(1,2,2,6,6-Pentamethyl-4-piperidyl)-6-(4,4,5,5-tetramethyl-1,3,2-dioxaborolan-2-yl)indazole

### Prepared by General Procedure Scheme S7 Step 2A

Yield: 1.8 g (58.77%); LCMS(ESI): [M+H]⁺ m/z: calcd 398.2; found 398.0; Rt=1.118 min.

### Step 4: The Synthesis of tert-Butyl (3S)-3-methyl-6-[2-(1,2,2,6,6-pentamethyl-4-piperidyl) indazol-6-yl]-3,4-dihydro-2H-pyridine-1-carboxylate

### Prepared by General Procedure Scheme S7 Step 2

Yield: 2.0 g (crude); LCMS(ESI): [M+H]⁺ m/z: calcd 467.2; found 467.4; Rt=1.276 min.

### Step 5: The Synthesis of 2-(1,2,2,6,6-Pentamethyl-4-piperidyl)-6-[(3S)-3-methyl-1,2,3,4-tetrahydropyridin-6-yl]indazole

### Prepared by General Procedure Scheme S7 Step 3 (Method A)

Yield: 0.9 g (57.29%); LCMS(ESI): [M+H]⁺ m/z: calcd 367.2; found 367.2; Rt=0.727 min.

### Step 6: The Synthesis of 2-(1,2,2,6,6-Pentamethyl-4-piperidyl)-6-[(2R,5S)-5-methyl-2-piperidyl]indazole

### Prepared by General Procedure Scheme S7 Step 4

Yield: 0.8 g (93.60%); LCMS(ESI): [M+H]⁺ m/z: calcd 369.2; found 369.2; Rt=0.767 min.

### Step 7: The Synthesis of 2-oxo-2-[(2R,5S)-5-methyl-2-[2-(1,2,2,6,6-pentamethyl-4-piperidyl) indazol-6-yl]-1-piperidyl]-N-[1-(2-trimethylsilylethoxymethyl)pyrazolo[4,3-c]pyridin-7-yl]acetamide

### Prepared by General Procedure Scheme S7 Step 5

HPLC (column: XBridge C18 100×19 mm 5 um; mobile phase: 40-90% 0-6 min H₂O/MeOH/0.1% NH₄OH, flow rate: 30 ml/min (loading pump 4 ml/min methanol))

Yield: 96.5 mg (25.89%); LCMS(ESI): [M+H]⁺ m/z: calcd 686.4; found 687.2; Rt=3.017 min.

### Step 8: The Synthesis of 2-oxo-N-(1H-pyrazolo[4,3-c]pyridin-7-yl)-2-[(2R,5S)-5-methyl-2-[2-(1,2,2,6,6-pentamethyl-4-piperidyl) indazol-6-yl]-1-piperidyl]acetamide (Compound 45)

2-oxo-2-[(2R,5S)-5-methyl-2-[2-(1,2,2,6,6-pentamethyl-4-piperidyl) indazol-6-yl]-1-piperidyl]-N-[1-(2-trimethylsilylethoxymethyl)pyrazolo[4,3-c]pyridin-7-yl]acetamide (96.5 mg, 140.47 μmol) was dissolved in a mixture of Diox/HCl (2 mL) and MeOH (2 mL), the resulting mixture was stirred at 25° C. for 16 hr. After that time, solvent was evaporated to dryness to afford crude product. Crude product was subjected by HPLC (column: Chromatorex 18 SMB100-5T 100×19 mm, 5 um; mobile phase: 0-40% 0-5 min H₂O/MeOH/0.1% FA, flow rate: 30 ml/min (loading pump 4 ml/min MeCN), affording 2-oxo-N-(1H-pyrazolo[4,3-c]pyridin-7-yl)-2-[(2R,5S)-5-methyl-2-[2-(1,2,2,6,6-pentamethyl-4-piperidyl) indazol-6-yl]-1-piperidyl]acetamide (52.6 mg, 83.54 μmol, 59.47% yield, 2HCl) on two portions.

¹H NMR (600 MHz, dmso) § 1.04-1.10 (m, 3H), 1.17-1.30 (m, 12H), 1.34-1.41 (m, 1H), 1.73-2.35 (m, 10H), 2.38-2.44 (m, 2H), 2.86-3.09 (m, 1H), 3.72-4.11 (m, 1H), 4.89-5.06 (m, 1H), 5.51-5.74 (m, 1H), 6.98-7.11 (m, 1H), 7.56 (s, 1H), 7.65-7.75 (m, 1H), 8.07-8.59 (m, 4H), 8.87-8.99 (m, 1H), 11.02-11.21 (m, 1H), 12.93-13.18 (m, 1H).

LCMS(ESI): [M+H]⁺ m/z: calcd 556.4; found 557.6; Rt=1.761 min.

### Example 92. Synthesis of N-(8-amino-2,7-naphthyridin-4-yl)-2-oxo-2-[(2R,5S)-5-methyl-2-[2-(1-methyl-4-piperidyl) indazol-6-yl]-1-piperidyl]acetamide (Compound 1)

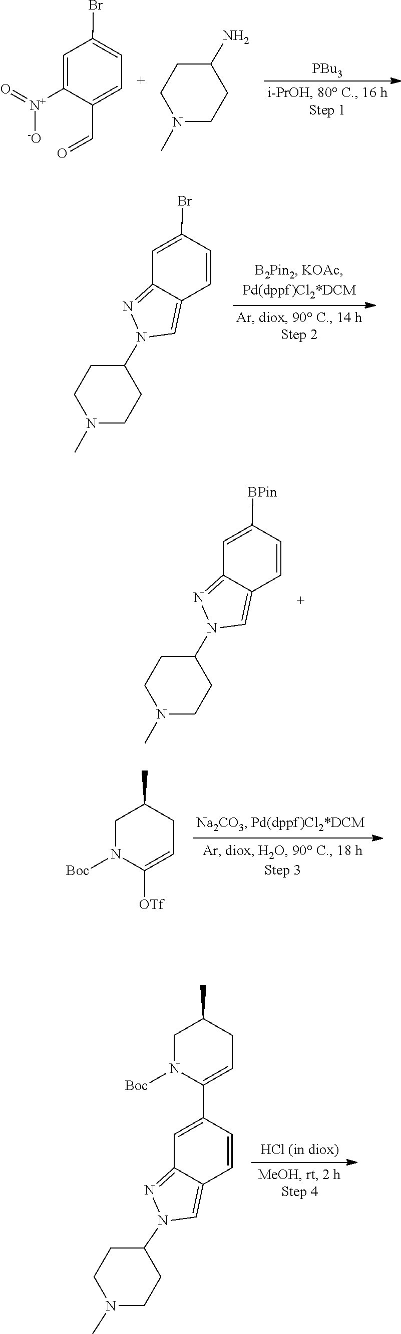

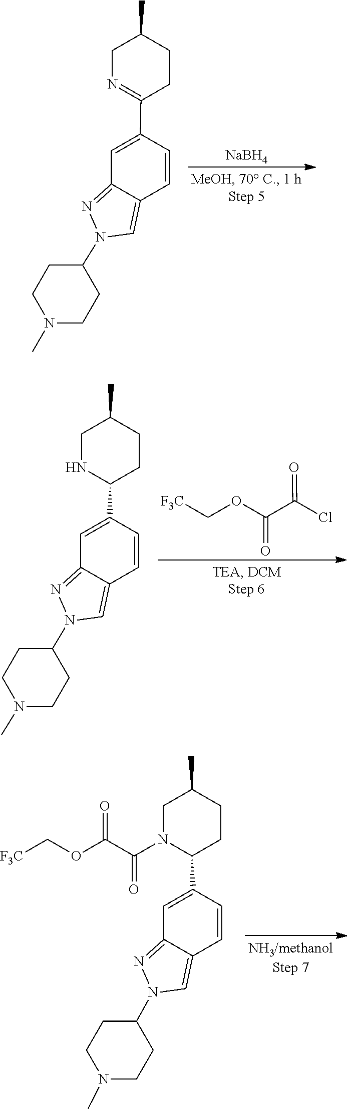 [SMILES: C[C@H]1CCC(c2ccc3cn(C4CCN(C)CC4)nc3c2)=NC1 ; "NaBH4" ; C[C@H]1CC[C@H](c2ccc3cn(C4CCN(C)CC4)nc3c2)NC1 ; O=C(Cl)C(=O)OCC(F)(F)F ; C[C@H]1CC[C@H](c2ccc3cn(C4CCN(C)CC4)nc3c2)N(C(=O)C(=O)OCC(F)(F)F)C1]

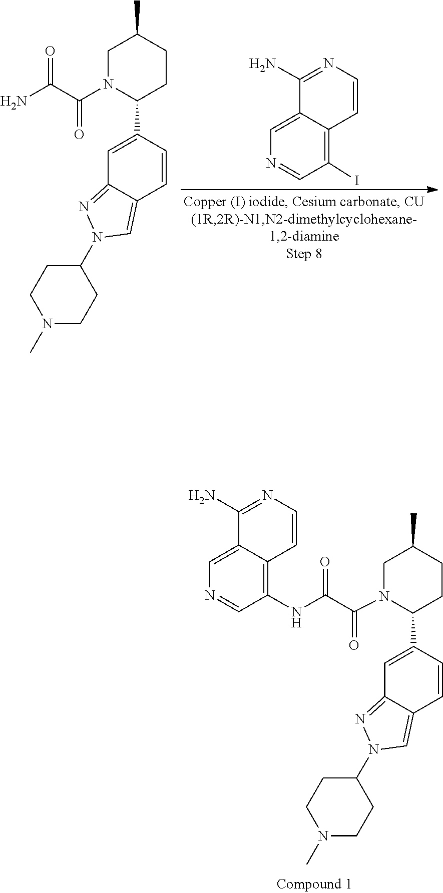 [SMILES: C[C@H]1CC[C@H](c2ccc3cn(C4CCN(C)CC4)nc3c2)N(C(=O)C(N)=O)C1 ; Nc1nccc2c(I)cncc12 ; C[C@H]1CC[C@H](c2ccc3cn(C4CCN(C)CC4)nc3c2)N(C(=O)C(=O)Nc2cncc3c(N)nccc23)C1]

### Step 1: The Synthesis of 6-Bromo-2-(1-methyl-4-piperidyl)indazole

### Prepared by General Procedure Scheme S7 Step 1A

FCC conditions: Interchim; 330 g SiO₂, MTBE-MeOH from 0˜100%, flow rate=100 mL/min, cv=10

Yield: 38.0 g (74.28%); LCMS(ESI): [M+2H]⁺ m/z: calcd 296.2; found 296.2; Rt=0.808 min.

### Step 2: The Synthesis of 2-(1-Methyl-4-piperidyl)-6-(4,4,5,5-tetramethyl-1,3,2-dioxaborolan-2-yl)indazole

### Prepared by General Procedure Scheme S7 Step 2A

FCC conditions: Interchim; 120 g SiO₂, MTBE-MeOH from 0˜100%, flow rate=70 mL/min, cv=7

Yield: 21.0 g (47.64%); LCMS(ESI): [M+H]⁺ m/z: calcd 342.2; found 342.0; Rt=1.045 min.

### Step 3: The Synthesis of tert-Butyl (3S)-3-methyl-6-[2-(1-methyl-4-piperidyl) indazol-6-yl]-3,4-dihydro-2H-pyridine-1-carboxylate

### Prepared by General Procedure Scheme S7 Step 2

FCC conditions: Interchim; 120 g SiO₂, MTBE-MeOH from 0˜100%, flow rate=70 mL/min, cv=11

Yield: 9.0 g (74.81%); LCMS(ESI): [M+H]⁺ m/z: calcd 411.2; found 411.4; Rt=1.165 min.

### Step 4: The Synthesis of 2-(1-Methyl-4-piperidyl)-6-[(3S)-3-methyl-2,3,4,5-tetrahydropyridin-6-yl]indazole

### Prepared by General Procedure Scheme S7 Step 3 (Method B)

Yield: 6.0 g (71.40%, 2HCl); LCMS(ESI): [M+H]⁺ m/z: calcd 311.2; found 311.2; Rt=0.578 min.

### Step 5: The Synthesis of 2-(1-Methyl-4-piperidyl)-6-[(2R,5S)-5-methyl-2-piperidyl]indazole

### Prepared by General Procedure Scheme S7 Step 4

Yield: 4.9 g (crude); LCMS(ESI): [M+H]⁺ m/z: calcd 313.2; found 313.2; Rt=0.543 min.

### Step 6: Synthesis of N-(8-amino-2,7-naphthyridin-4-yl)-2-oxo-2-[(2R,5S)-5-methyl-2-[2-(1-methyl-4-piperidyl) indazol-6-yl]-1-piperidyl]acetamide

2-(1-methyl-4-piperidyl)-6-[(2R,5S)-5-methyl-2-piperidyl]indazole (2 g, 5.76 mmol) and TEA (582.95 mg, 5.76 mmol, 802.95 μL) was dissolved in DCM (39.67 mL), cooled with ice-water bath and then ethyl 2-chloro-2-oxo-acetate (825.88 mg, 6.05 mmol, 676.95 μL) in 20 ml of DCM was added dropwise in 10 min. Solution was stirred overnight. Reaction mixture was washed with aqueous solution of NaHCO₃, dried over sodium sulphate and evaporated to give ethyl 2-oxo-2-[(2R,5S)-5-methyl-2-[2-(1-methyl-4-piperidyl) indazol-6-yl]-1-piperidyl]acetate (1.2 g, 2.91 mmol, 50.49% yield). LCMS(ESI): [M+H]⁺ m/z: calcd 412.2; found 413.2; Rt=1.025 min.

### Step 7: Synthesis of 2-oxo-2-[(2R,5S)-5-methyl-2-[2-(1-methyl-4-piperidyl) indazol-6-yl]-1-piperidyl]acetamide

ethyl 2-oxo-2-[(2S,5R)-5-methyl-2-[2-(1-methyl-4-piperidyl) indazol-6-yl]-1-piperidyl]acetate (1.2 g, 2.91 mmol) was dissolved in NH₃/methanol (30 mL) solution (10% by weight) and stirred overnight, then concentrated in vacuo to give 2-oxo-2-[(2R,5S)-5-methyl-2-[2-(1-methyl-4-piperidyl) indazol-6-yl]-1-piperidyl]acetamide (0.7 g, 1.83 mmol, 62.75% yield). LCMS(ESI): [M+H]⁺ m/z: calcd 383.2; found 384.2; Rt=0.780 min.

### Step 7: Synthesis of N-(8-amino-2,7-naphthyridin-4-yl)-2-oxo-2-[(2R,5S)-5-methyl-2-[2-(1-methyl-4-piperidyl) indazol-6-yl]-1-piperidyl]acetamide (Compound 1)

5-iodo-2,7-naphthyridin-1-amine (134.30 mg, 495.45 μmol), 2-oxo-2-[(2R,5S)-5-methyl-2-[2-(1-methyl-4-piperidyl) indazol-6-yl]-1-piperidyl]acetamide (0.19 g, 495.45 μmol), Copper (I) iodide (94.36 mg, 495.45 μmol, 16.79 μL), Cesium carbonate (322.86 mg, 990.91 μmol) and (1R,2R)—N1,N2-dimethylcyclohexane-1,2-diamine (105.71 mg, 743.18 μmol) were mixed in Dioxane (7.99 mL) under argon, and then stirred overnight at 100° C. for 12 hr in vial. Reaction mixture was filtered, then evaporated. Crude product was dissolved in 2 ml DMSO and subjected to HPLC to give N-(8-amino-2,7-naphthyridin-4-yl)-2-oxo-2-[(2R,5S)-5-methyl-2-[2-(1-methyl-4-piperidyl) indazol-6-yl]-1-piperidyl]acetamide (7 mg, 13.29 μmol, 2.68%)

Chromatography data: Device (Mobile Phase, Column): SYSTEM 2-10 min 45-60% methanol+nh3 flow 30 ml/min (loading pump 4 mlmethanol), column: sun fire c18

### Example 93. The synthesis of 2-((2R,5S)-2-(benzo[d]thiazol-5-yl)-5-methylpiperidin-1-yl)-2-oxo-N-(1H-pyrazolo[4,3-c]pyridin-7-yl)acetamide (Compound 26)

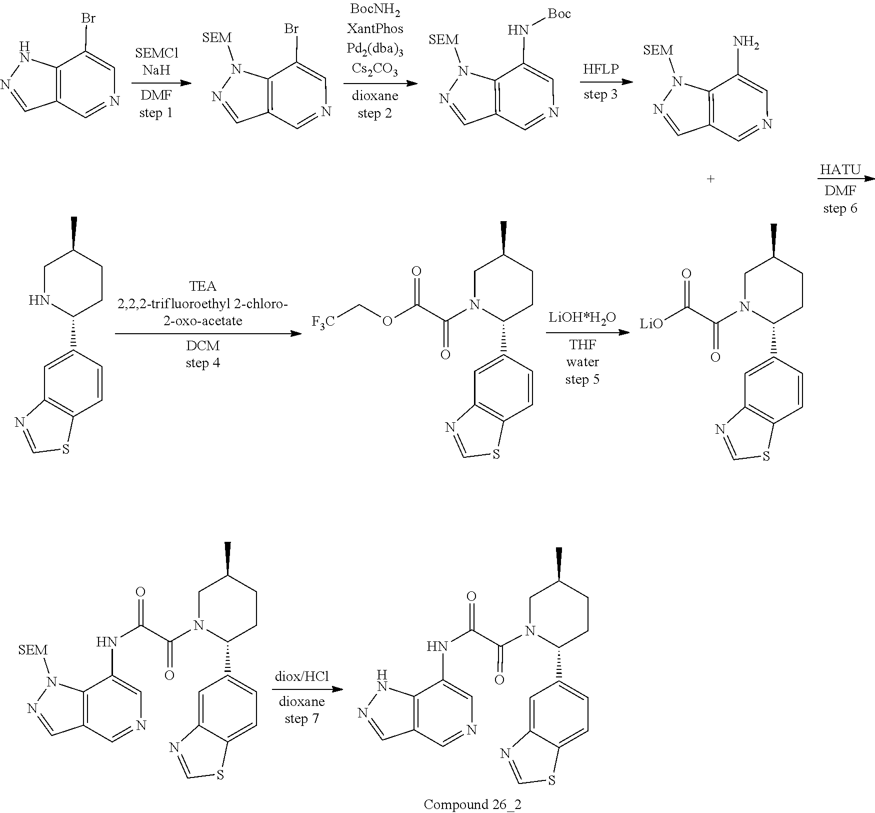

### Step 1: Synthesis of 7-bromo-1-((2-(trimethylsilyl)ethoxy)methyl)-1H-pyrazolo[4,3-c]pyridine

Sodium hydride (in oil dispersion)₆₀% dispersion in mineral oil (212.85 mg, 5.55 mmol, 60% purity) was added portion wise to a solution of 7-bromo-1H-pyrazolo[4,3-c]pyridine (1 g, 5.05 mmol) in DMF (20 mL) at 0° C. When H₂ evolution ceased, 2-(chloromethoxy)ethyl-trimethyl-silane (884.04 mg, 5.30 mmol, 938.47 μL) was added dropwise. Resulting mixture was stirred at 0° C. for 3 hr. Then, it was diluted with water (60 ml) and extracted with MTBE (50 ml). Organic layer was washed successively with water (20 ml) and brine (20 ml), dried over Na₂SO₄ and concentrated under reduced pressure, affording 2-[(7-bromopyrazolo[4,3-c]pyridin-1-yl) methoxy]ethyl-trimethyl-silane (1.6 g, 4.87 mmol, 96.51% yield). LCMS(ESI): [M]⁺ m/z: calcd 328.2; found 329.2; Rt=1.458 min.

### Step 2: Synthesis of tert-butyl (1-((2-(trimethylsilyl)ethoxy)methyl)-1H-pyrazolo[4,3-c]pyridin-7-yl)carbamate

2-[(7-Bromopyrazolo[4,3-c]pyridin-1-yl) methoxy]ethyl-trimethyl-silane (2.2 g, 6.70 mmol), tert-butyl carbamate (1.18 g, 10.05 mmol) and cesium carbonate (3.28 g, 10.05 mmol) were mixed together in dioxane (40 mL). Reaction flask was evacuated and refilled with argon 3 times. Then, Pd₂(dba)₃ (153.42 mg, 167.54 μmol) and Xantphos (193.88 mg, 335.08 μmol) were added under stream of Ar. Resulting mixture was stirred at 100° C. for 16 hr. Then, it was cooled, diluted with MTBE (50 ml) and filtered through a short pad of silica gel. Filtrate was concentrated under reduced pressure, affording tert-butyl N-[1-(2-trimethylsilylethoxymethyl)pyrazolo[4,3-c]pyridin-7-yl]carbamate (3.1 g, crude).

LCMS(ESI): [M]⁺ m/z: calcd 364.2; found 365.2; Rt=1.374 min.

### Step 3: Synthesis of 1-((2-(trimethylsilyl)ethoxy)methyl)-1H-pyrazolo[4,3-c]pyridin-7-amine

tert-Butyl N-[1-(2-trimethylsilylethoxymethyl)pyrazolo[4,3-c]pyridin-7-yl]carbamate (3.1 g, 8.50 mmol) was dissolved in HFIP (24.00 g, 142.82 mmol, 15 mL). Resulting solution was stirred at 58° C. for 96 hr. Then, solvent was removed under reduced pressure, affording 1-(2-trimethylsilylethoxymethyl)pyrazolo[4,3-c]pyridin-7-amine (2.7 g, crude). LCMS(ESI): [M]⁺ m/z: calcd 264.2; found 265.2; Rt=1.093 min.

### Step 4: Synthesis of 2,2,2-trifluoroethyl 2-((2R,5S)-2-(benzo[d]thiazol-5-yl)-5-methylpiperidin-1-yl)-2-oxoacetate

2,2,2-Trifluoroethyl 2-chloro-2-oxo-acetate (301.08 mg, 1.58 mmol) was added dropwise to a solution of 5-[(2R,5S)-5-methyl-2-piperidyl]-1,3-benzothiazole (360 mg, 1.55 mmol) and TEA (235.18 mg, 2.32 mmol, 323.94 μL) in DCM (20 mL) at 0° C. After addition was complete, cooling bath was removed and resulting mixture was allowed to warm up to 20° C. and stirred for 1 hr. Then, it was quenched with water (20 ml). After 5 min stirring, organic layer was separated, dried over Na₂SO₄ and concentrated under reduced pressure, affording 2,2,2-trifluoroethyl 2-[(2R,5S)-2-(1,3-benzothiazol-5-yl)-5-methyl-1-piperidyl]-2-oxo-acetate (590 mg, 1.53 mmol, 98.55% yield). LCMS(ESI): [M]⁺ m/z: calcd 386.2; found 387.2; Rt=1.412 min.

### Step 5: Synthesis of lithium 2-((2R,5S)-2-(benzo[d]thiazol-5-yl)-5-methylpiperidin-1-yl)-2-oxoacetate

To a solution of 2,2,2-trifluoroethyl 2-[(2R,5S)-2-(1,3-benzothiazol-5-yl)-5-methyl-1-piperidyl]-2-oxo-acetate (5.05 g, 13.07 mmol) in THF (50 mL) was added lithium hydroxide monohydrate, 98% (548.45 mg, 13.07 mmol, 363.21 μL) as a solution in water (10 mL) and the resulting mixture was left to stir at rt for 1 hr. Then the resulting mixture was evaporated to dryness, dissolved in water, washed with DCM three times. Water was acidified to pH=1 and extracted with EtOAc twice, organics were washed with brine, dried over Na₂SO₄, and evaporated. Lithium 2-[(2R,5S)-2-(1,3-benzothiazol-5-yl)-5-methyl-1-piperidyl]-2-oxo-acetate (4.47 g, crude) was used as a lithium salt in the next steps without further purification.

LCMS(ESI): [M]⁺ m/z: calcd 304.2; found 305.2; Rt=1.347 min.

### Step 6: Synthesis of 2-((2R,5S)-2-(benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

1-(2-Trimethylsilylethoxymethyl)pyrazolo[4,3-c]pyridin-7-amine (0.27 g, 1.02 mmol) HATU (407.70 mg, 1.07 mmol) were mixed in dry DMF (5 mL) at rt and the resulting mixture was stirred for 15 min. Lithium-2-[(2R,5S)-2-(1,3-benzothiazol-5-yl)-5-methyl-1-piperidyl]-2-oxo-acetate (316.87 mg, 1.02 mmol) was added thereto and the resulting mixture was stirred at rt overnight. The resulting mixture was poured into water, extracted 3 times with EtOAc, combined organics were washed with water, brine and evaporated. The residue was subjected to HPLC (0.5-6.5 min 73% water-MeOH, 30 ml/min (loading pump 4 ml MeOH); column: SunFire 100*19 mm, 5 microM). 2-[(2R,5S)-2-(1,3-Benzothiazol-5-yl)-5-methyl-1-piperidyl]-2-oxo-N-[1-(2-trimethylsilylethoxymethyl)pyrazolo[4,3-c]pyridin-7-yl]acetamide (105.4 mg, 191.38 μmol, 18.74% yield) was obtained as a brown solid. LCMS(ESI): [M]⁺ m/z: calcd 550.2; found 551.2; Rt=4.100 min.

### Step 7: Synthesis of 2-((2R,5S)-2-(benzo[d]thiazol-5-yl)-5-methylpiperidin-1-yl)-2-oxo-N-(1H-pyrazolo[4,3-c]pyridin-7-yl)acetamide (Compound 26)

To a solution of 2-[(2R,5S)-2-(1,3-benzothiazol-5-yl)-5-methyl-1-piperidyl]-2-oxo-N-[1-(2-trimethylsilylethoxymethyl)pyrazolo[4,3-c]pyridin-7-yl]acetamide (105.4 mg, 191.38 μmol) in dioxane (2 mL) was added hydrogen chloride solution 4.0M in dioxane (1.84 g, 50.55 mmol, 2.30 mL) at 21° C. The resulting mixture was left to stir for 72 hr. The resulting mixture was evaporated to dryness and subjected to HPLC (0.5-6.5 min 20-45% water-MeCN+NH₃, 30 ml/min (loading pump 4 ml/MeCN), column: YMC-ACTUS TRIART C18 100*20 5 microM). The residue was further purified on chiral column.

Analytical: Chiralpak AD-H (250*4.6 mm, 5 mkm), IPA-MeOH, 50-50, 0.6 ml/min-RT=50.736 min.

Preparative: Chiralpak AD-H (250*20 mm, 5 mkm), MeOH-IPA, 50-50, 10 ml/min-RT=53.968 min.

2-[(2R,5S)-2-(1,3-Benzothiazol-5-yl)-5-methyl-1-piperidyl]-2-oxo-N-(1H-pyrazolo[4,3-c]pyridin-7-yl)acetamide (23.41 mg, 55.67 μmol, 29.09% yield) was obtained as a beige solid.

Compound 26: ¹H NMR (600 MHZ, DMSO-d₆) δ (ppm) 1.04-1.11 (m, 3H), 1.34-1.45 (m, 1H), 1.72-1.81 (m, 1H), 1.88-1.99 (m, 1H), 2.14-2.27 (m, 1H), 2.27-2.36 (m, 1H), 2.87-3.05 (m, 1H), 3.75-4.20 (m, 1H), 5.58-5.85 (m, 1H), 7.44-7.54 (m, 1H), 7.99-8.09 (m, 1H), 8.12-8.22 (m, 1H), 8.35-8.45 (m, 1H), 8.45-8.62 (m, 1H), 8.93-9.08 (m, 1H), 9.35-9.46 (m, 1H), 11.09-11.29 (m, 1H), 13.07-13.31 (m, 1H). LCMS(ESI): [M]⁺ m/z: calcd 420.2; found 421.2; Rt=2.388 min.

### Example 94. The synthesis of 2-((2R,5S)-2-(benzo[d]thiazol-5-yl)-5-methylpiperidin-1-yl)-2-oxo-N-(1H-pyrazolo[4,3-c]pyridin-7-yl)acetamide (Compound 26)

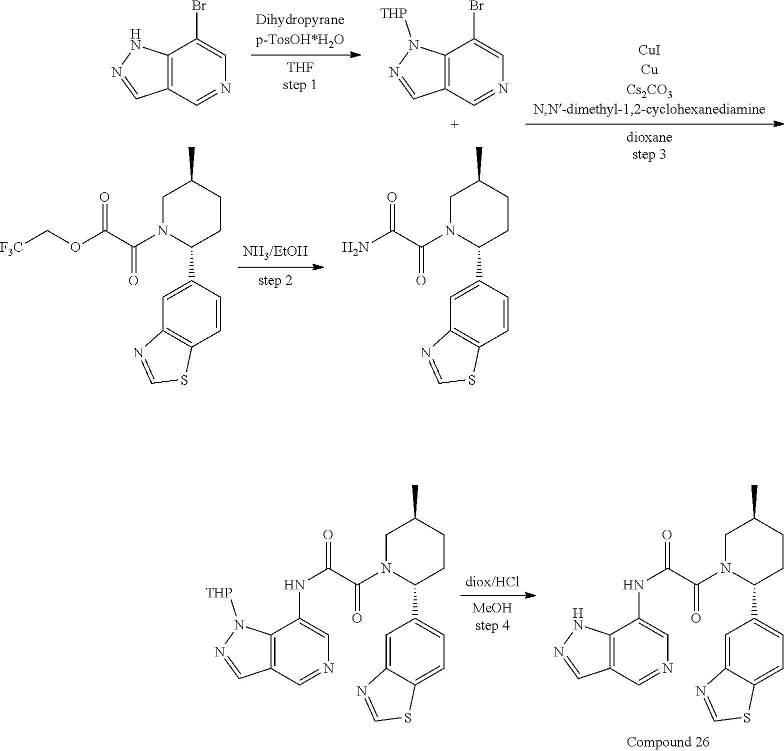

### Step 1: Synthesis of 7-bromo-1-(tetrahydro-2H-pyran-2-yl)-1H-pyrazolo[4,3-c]pyridine

To a solution of 7-bromo-1H-pyrazolo[4,3-c]pyridine (5 g, 25.25 mmol), dihydropyrane (6.37 g, 75.75 mmol, 6.88 mL) and p-toluenesulfonic acid monohydrate (480.31 mg, 2.52 mmol, 387.34 μL) in THF (50 mL) was stirred at 65° C. for 36 hr and evaporated in vacuum. The residue (11 g) was purified by gradient chromatography (CHCl₃-MeCN) to obtain 7-bromo-1-tetrahydropyran-2-yl-pyrazolo[4,3-c]pyridine (4.5 g, 15.95 mmol, 63.17% yield).

LCMS(ESI): [M]⁺ m/z: calcd 282.2; found 283.2; Rt=0.714 min.

### Step 2: Synthesis of 2-((2R,5S)-2-(benzo[d]thiazol-5-yl)-5-methylpiperidin-1-yl)-2-oxoacetamide

2,2,2-Trifluoroethyl 2-[(2R,5S)-2-(1,3-benzothiazol-5-yl)-5-methyl-1-piperidyl]-2-oxo-acetate (Example 93)(590 mg, 1.53 mmol) was dissolved in ammonia, 2M in EtOH (7.85 g, 19.96 mmol, 10 mL, 4.33% purity). Resulting solution was stirred at 25° C. for 15 hr. Then, volatiles were removed under reduced pressure, leaving 2-[(2R,5S)-2-(1,3-benzothiazol-5-yl)-5-methyl-1-piperidyl]-2-oxo-acetamide (437 mg, 1.44 mmol, 94.33% yield). LCMS(ESI): [M]⁺ m/z: calcd 303.2; found 304.2; Rt=1.104 min.

### Step 3: Synthesis of 2-((2R,5S)-2-(benzo[d]thiazol-5-yl)-5-methylpiperidin-1-yl)-2-oxo-N-(1-(tetrahydro-2H-pyran-2-yl)-1H-pyrazolo[4,3-c]pyridin-7-yl)acetamide

2-[(2R,5S)-2-(1,3-Benzothiazol-5-yl)-5-methyl-1-piperidyl]-2-oxo-acetamide (165.00 mg, 543.87 μmol) was added to mixture of 7-bromo-1-tetrahydropyran-2-yl-pyrazolo[4,3-c]pyridine (0.15 g, 531.66 μmol), copper (16.89 mg, 265.83 μmol), copper (I) iodide (10.13 mg, 53.17 μmol, 1.80 μL) and cesium carbonate (346.45 mg, 1.06 mmol) in dioxane (15 mL). The resulting mixture was allowed to stir at 100° C. for 2 hr. Then reaction mixture was filtered through the celite and evaporated. LCMS(ESI): [M]⁺ m/z: calcd 504.2; found 505.2; Rt=2.781 min.

### Step 4: Synthesis of 2-((2R,5S)-2-(benzo[d]thiazol-5-yl)-5-methylpiperidin-1-yl)-2-oxo-N-(1H-pyrazolo[4,3-c]pyridin-7-yl)acetamide (Compound 26)

To a solution of 2-[(2R,5S)-2-(1,3-benzothiazol-5-yl)-5-methyl-1-piperidyl]-2-oxo-N-(1-tetrahydropyran-2-ylpyrazolo[4,3-c]pyridin-7-yl)acetamide (250 mg, 495.44 μmol) in MeOH (3 mL) was added hydrogen chloride solution 4.0M in dioxane (2.40 g, 65.82 mmol, 3 mL) at 21° C. The resulting mixture was left to stir for 15 min. The resulting mixture was evaporated to dryness and subjected to HPLC (10-400.5-6.5 min water-MeCN+NH₃; flow 30 ml/min (loading pump 4 ml/min MeCN); column XBridge 100×19 mm 5 um (R)). 41.9 mg of 84.06% purity has been obtained. The residue was further purified on chiral columns (Chiralpak AD-H (250*30, 10 mkm), IPA-MeOH, 50-50, 25 ml/min).

Analytical: RT for Compound 26 (Chiralpak AD-H (250*4.6 mm, 5 mkm), IPA-MeOH, 50-50, 0.5 ml/min)—57.666 min.

Preparative: RT for Compound 26 (Chiralpak AD-H (250*30, 10 mkm), IPA-MeOH, 50-50, 25 ml/min)—50.403 min.

Optical rotatory power=+143.86°, +143.77° (0.075 g/100 ml, MeOH, 21° C.)

Compound 26: ¹H NMR (600 MHZ, DMSO-d₆) δ (ppm) 1.04-1.11 (m, 3H), 1.34-1.45 (m, 1H), 1.72-1.81 (m, 1H), 1.88-1.99 (m, 1H), 2.14-2.27 (m, 1H), 2.27-2.36 (m, 1H), 2.87-3.05 (m, 1H), 3.75-4.20 (m, 1H), 5.58-5.85 (m, 1H), 7.44-7.54 (m, 1H), 7.99-8.09 (m, 1H), 8.12-8.22 (m, 1H), 8.35-8.45 (m, 1H), 8.45-8.62 (m, 1H), 8.93-9.08 (m, 1H), 9.35-9.46 (m, 1H), 11.09-11.29 (m, 1H), 13.07-13.31 (m, 1H). LCMS(ESI): [M]⁺ m/z: calcd 420.2; found 421.2; Rt=2.388 min.

### Example 95. Synthesis of 2-(5-methyl-2-(2-methyl-2H-indazol-6-yl)piperidin-1-yl)-2-oxo-N-(1H-pyrazolo[4,3-c]pyridin-7-yl)acetamide Compound 113 and Compound 13)

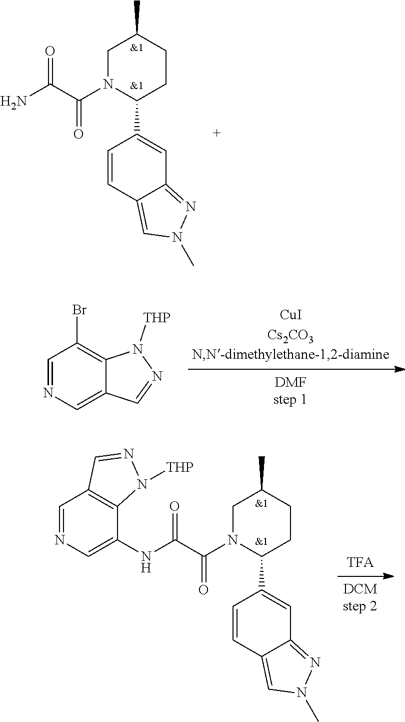

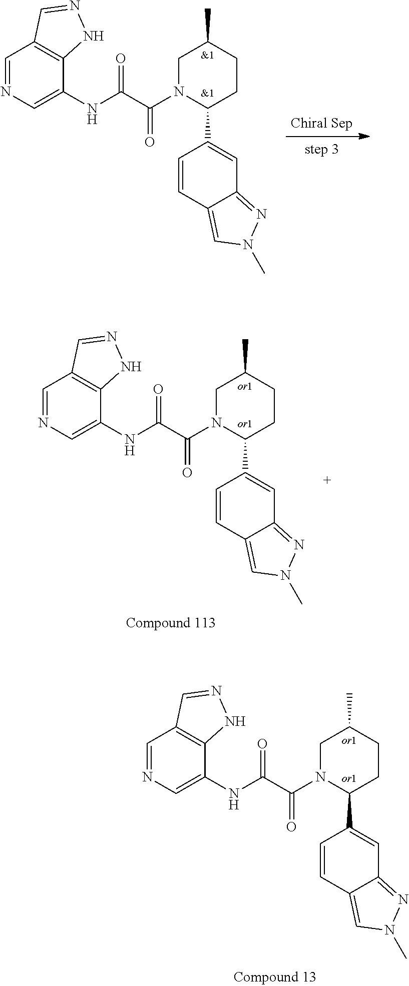 [SMILES: C[C@H]1CC[C@H](c2ccc3cn(C)nc3c2)N(C(=O)C(=O)Nc2cncc3cn[nH]c23)C1 ; C[C@H]1CC[C@H](c2ccc3cn(C)nc3c2)N(C(=O)C(=O)Nc2cncc3cn[nH]c23)C1 ; C[C@@H]1CC[C@@H](c2ccc3cn(C)nc3c2)N(C(=O)C(=O)Nc2cncc3cn[nH]c23)C1]

The synthesis of the starting 2-[(2R,5S)-5-methyl-2-(2-methylindazol-6-yl)-1-piperidyl]-2-oxo-acetamide was described in Intermediate 29.

### Step 1: Synthesis of rac-2-((2R,5S)-5-methyl-2-(2-methyl-2H-indazol-6-yl)piperidin-1-yl)-2-oxo-N-(1-(tetrahydro-2H-pyran-2-yl)-1H-pyrazolo[4,3-c]pyridin-7-yl)acetamide

To a mixture of 2-[rac-(2R,5S)-5-methyl-2-(2-methylindazol-6-yl)-1-piperidyl]-2-oxo-acetamide (212.91 mg, 708.88 μmol), copper (I) iodide (27.00 mg, 141.78 μmol, 4.80 μL), cesium carbonate (461.93 mg, 1.42 mmol) and N,N′-dimethylethane-1,2-diamine (12.50 mg, 141.78 μmol, 15.28 μL) in DMF (4 mL) 7-bromo-1-tetrahydropyran-2-yl-pyrazolo[4,3-c]pyridine (0.2 g, 708.88 μmol) was added in one portion. The resulting mixture was allowed to stir at 130° C. for 30 hr. Upon completion of the reaction, the mixture was subjected to HPLC 2 times to afford 2-[rac-(2R,5S)-5-methyl-2-(2-methylindazol-6-yl)-1-piperidyl]-2-oxo-N-(1-tetrahydropyran-2-ylpyrazolo[4,3-c]pyridin-7-yl)acetamide (0.052 g, 103.67 μmol, 14.62% yield). LCMS(ESI): [M]⁺ m/z: calcd 501.2; found 502.2; Rt=2.793 min.

### Step 2: Synthesis of rac-2-((2R,5S)-5-methyl-2-(2-methyl-2H-indazol-6-yl)piperidin-1-yl)-2-oxo-N-(1H-pyrazolo[4,3-c]pyridin-7-yl)acetamide

To the mixture of DCM (1 mL) and TFA (1 mL), 2-[rac-(2R,5S)-5-methyl-2-(2-methylindazol-6-yl)-1-piperidyl]-2-oxo-N-(1-tetrahydropyran-2-ylpyrazolo[4,3-c]pyridin-7-yl)acetamide (0.052 g, 103.67 μmol) was added in one portion, resulting mixture was stirred at rt overnight. Upon completion of the reaction, resulting mixture was poured into ice water, stirred for 10 min and extracted with DCM (3*10 ml), combined organic layers was dried and evaporated to obtain 2-[rac-(2R,5S)-5-methyl-2-(2-methylindazol-6-yl)-1-piperidyl]-2-oxo-N-(1H-pyrazolo[4,3-c]pyridin-7-yl)acetamide (0.04 g, 95.82 μmol, 92.42% yield). LCMS(ESI): [M]⁺ m/z: calcd 417.2; found 418.2; Rt=0.773 min.

### Step 3: Chiral Separation (Compound 113 and Compound 13)

Racemic 2-[rac-(2R,5S)-5-methyl-2-(2-methylindazol-6-yl)-1-piperidyl]-2-oxo-N-(1H-pyrazolo[4,3-c]pyridin-7-yl)acetamide (0.04 g, 95.82 μmol) was chiral separated (Column: Chiralcel OD-H (250*20, 5 mkm), Hexane-IPA-MeOH, 70-15-15, 14 ml/min) to obtain 2-[rel-(2S,5R)-5-methyl-2-(2-methylindazol-6-yl)-1-piperidyl]-2-oxo-N-(1H-pyrazolo[4,3-c]pyridin-7-yl)acetamide (0.00863 g, 20.67 μmol, 21.58% yield) (RT=14.09 min) and 2-[rel-(2R,5S)-5-methyl-2-(2-methylindazol-6-yl)-1-piperidyl]-2-oxo-N-(1H-pyrazolo[4,3-c]pyridin-7-yl)acetamide (0.00762 g, 18.25 μmol, 19.05% yield) (RT=18.52 min).

Rel Time for Compound 113 in analytical conditions (column: OD-H, Hexane-IPA-MeOH, 50-25-25, 0.6 ml/min as mobile phase) 19.63 min and for Compound 13 16.20 min.

Compound 113: Retention time: 19.63 min. ¹H NMR (600 MHZ, DMSO-d₆) δ (ppm) 1.03-1.09 (m, 3H), 1.32-1.45 (m, 1H), 1.74-1.83 (m, 1H), 1.86-1.98 (m, 1H), 2.08-2.24 (m, 1H), 2.26-2.35 (m, 1H), 2.85-3.26 (m, 1H), 3.76-4.11 (m, 1H), 4.12-4.20 (m, 3H), 5.45-5.75 (m, 1H), 6.98-7.09 (m, 1H), 7.53 (s, 1H), 7.64-7.74 (m, 1H), 8.22-8.37 (m, 2H), 8.43-8.58 (m, 1H), 8.88-9.01 (m, 1H), 10.92-11.25 (m, 1H), 12.87-13.19 (m, 1H).

LCMS(ESI): [M]⁺ m/z: calcd 417.2; found 418.2; Rt=2.106 min.

Compound 13: Retention time: 30.29 min. ¹H NMR (600 MHZ, DMSO-d₆) δ (ppm) 1.02-1.09 (m, 3H), 1.31-1.46 (m, 1H), 1.71-1.86 (m, 1H), 1.86-2.00 (m, 1H), 2.06-2.22 (m, 1H), 2.23-2.32 (m, 1H), 2.85-3.26 (m, 1H), 3.74-4.11 (m, 1H), 4.11-4.20 (m, 3H), 5.44-5.82 (m, 1H), 6.93-7.14 (m, 1H), 7.49-7.57 (m, 1H), 7.63-7.79 (m, 1H), 8.21-8.39 (m, 2H), 8.41-8.60 (m, 1H), 8.86-9.00 (m, 1H), 10.91-11.27 (m, 1H), 12.87-13.20 (m, 1H). LCMS(ESI): [M]⁺ m/z: calcd 417.2; found 418.2; Rt=2.115 min.

Stereochemistry was assigned based on assay activity.

### Example 96. Synthesis of N-(4-amino-1H-pyrazolo[4,3-c]pyridin-7-yl)-2-(2-(4-fluorophenyl)-4-methoxy-5-methylpiperidin-1-yl)-2-oxoacetamide (Compound 38 and Compound 7)

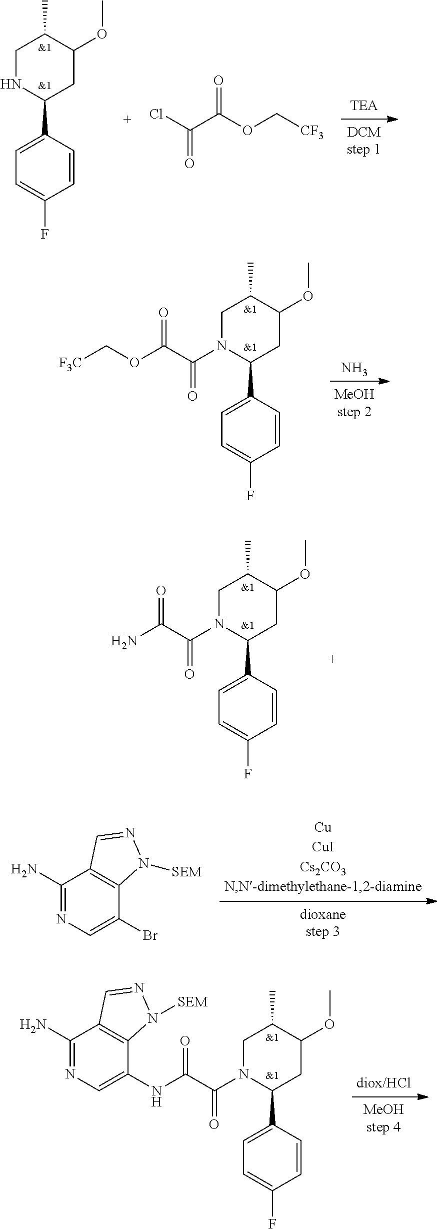

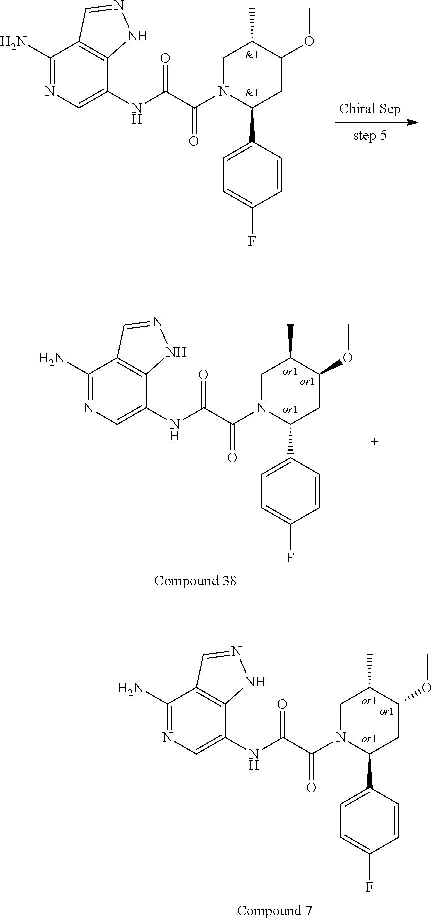 [SMILES: COC1C[C@@H](c2ccc(F)cc2)N(C(=O)C(=O)Nc2cnc(N)c3cn[nH]c23)C[C@@H]1C ; CO[C@H]1C[C@H](c2ccc(F)cc2)N(C(=O)C(=O)Nc2cnc(N)c3cn[nH]c23)C[C@H]1C ; CO[C@@H]1C[C@@H](c2ccc(F)cc2)N(C(=O)C(=O)Nc2cnc(N)c3cn[nH]c23)C[C@@H]1C]

The synthesis of rac-(2R,5R)-2-(4-Fluorophenyl)-4-methoxy-5-methyl-piperidine was described in Intermediate 30.

### Step 1: Synthesis of rac-2,2,2-trifluoroethyl 2-((2S,5S)-2-(4-fluorophenyl)-4-methoxy-5-methylpiperidin-1-yl)-2-oxoacetate

2,2,2-Trifluoroethyl 2-chloro-2-oxo-acetate (1.00 g, 5.26 mmol) was added dropwise to a solution of rac-(2S,5S)-2-(4-fluorophenyl)-4-methoxy-5-methyl-piperidine (1.30 g, 5.00 mmol, HCl) and TEA (1.52 g, 15.01 mmol, 2.09 mL) in DCM (25.00 mL) at 0° C. After addition was complete, cooling bath was removed and resulting mixture was allowed to warm up to 20° C. and stirred for 16 hr. Then, it was washed with water, dried over Na₂SO₄ and concentrated under reduced pressure, affording 2,2,2-trifluoroethyl 2-oxo-2-[rac-(2S,5S)-2-(4-fluorophenyl)-4-methoxy-5-methyl-1-piperidyl]acetate (1.7 g, crude) which was used in the next step without further purification. LCMS(ESI): [M+1]⁺ m/z: calcd 377.2; found 378.2; Rt=1.463 min.

### Step 2: Synthesis of rac-2-((2S,5S)-2-(4-fluorophenyl)-4-methoxy-5-methylpiperidin-1-yl)-2-oxoacetamide

A solution of 2,2,2-trifluoroethyl 2-oxo-2-[rac-(2S,5S)-2-(4-fluorophenyl)-4-methoxy-5-methyl-1-piperidyl]acetate (1.70 g, 4.51 mmol) in MeOH/NH₃ (5N)(25 mL) was stirred at 20° C. for 16 hr. The solvent was evaporated to obtain 2-oxo-2-[rac-(2S,5S)-2-(4-fluorophenyl)-4-methoxy-5-methyl-1-piperidyl]acetamide (1.28 g, crude). LCMS(ESI): [M+1]⁺ m/z: calcd 294.2; found 295.2; Rt=1.088 min.

### Step 3: Synthesis of rac-N-(4-amino-1-((2-(trimethylsilyl)ethoxy)methyl)-1H-pyrazolo[4,3-c]pyridin-7-yl)-2-((2S,5S)-2-(4-fluorophenyl)-4-methoxy-5-methylpiperidin-1-yl)-2-oxoacetamide

2-Oxo-2-[rac-(2S,5S)-2-(4-fluorophenyl)-4-methoxy-5-methyl-1-piperidyl]acetamide (115.00 mg, 390.73 μmol), 7-bromo-1-(2-trimethylsilylethoxymethyl)pyrazolo[4,3-c]pyridin-4-amine (134.14 mg, 390.73 μmol), copper (I) iodide (74.41 mg, 390.73 μmol, 13.24 μL), cesium carbonate (254.62 mg, 781.46 μmol) and N1,N2-dimethylethane-1,2-diamine (83.37 mg, 586.10 μmol) were mixed in dioxane (6 mL) under argon, and then stirred overnight at 100° C. for 15 hr in vial. The reaction mixture was filtered and the filtrate was concentrated in vacuum and the residue was purified by HPLC (2-10 min 0-50% MeCN+FA 30/min; loading pump 4 ml/min MeCN; column SunFire 19*100 mm) to give N-[4-amino-1-(2-trimethylsilylethoxymethyl)pyrazolo[4,3-c]pyridin-7-yl]-2-oxo-2-[rac-(2S,5S)-2-(4-fluorophenyl)-4-methoxy-5-methyl-1-piperidyl]acetamide (54.5 mg, 90.42 μmol, 23.14% yield, HCOOH). LCMS(ESI): [M+1]⁺ m/z: calcd 556.2; found 557.2; Rt=1.347 min.

### Step 4: Synthesis of rac-N-(4-amino-1H-pyrazolo[4,3-c]pyridin-7-yl)-2-((2S,5S)-2-(4-fluorophenyl)-4-methoxy-5-methylpiperidin-1-yl)-2-oxoacetamide

To a solution of N-[4-amino-1-(2-trimethylsilylethoxymethyl)pyrazolo[4,3-c]pyridin-7-yl]-2-oxo-2-[rac-(2S,5S)-2-(4-fluorophenyl)-4-methoxy-5-methyl-1-piperidyl]acetamide (54.50 mg, 97.90 μmol) in MeOH (5 mL) was added hydrogen chloride solution 4.0M in dioxane (800.00 mg, 21.94 mmol, 1 mL) at 26° C. The resulting mixture was left to stir for 14 hr. The resulting mixture was evaporated to dryness and then submitted to reverse phase HPLC (SYSTEM 2-10 min 0-60% MeOH+FA flow 30 ml/min (loading pump 4 ml MeOH), column: SunFire C18) to afford N-(4-amino-1H-pyrazolo[4,3-c]pyridin-7-yl)-2-oxo-2-[rac-(2S,5S)-2-(4-fluorophenyl)-4-methoxy-5-methyl-1-piperidyl]acetamide (21 mg, 44.45 μmol, 45.40% yield, HCOOH). LCMS(ESI): [M+1]⁺ m/z: calcd 426.2; found 427.2; Rt=1.016 min.

### Step 5: Chiral Separation (Compound 38 and Compound 7)

Racemic N-(4-amino-1H-pyrazolo[4,3-c]pyridin-7-yl)-2-oxo-2-[rac-(2S,5S)-2-(4-fluorophenyl)-4-methoxy-5-methyl-1-piperidyl]acetamide (10 mg, 21.17 μmol, HCOOH) was chiral separated (Column: Chiralcel OJ-H (250*20, 5 mkm); Hexane-IPA-MeOH; 50-25-25; 12 ml/min) to obtain N-(4-amino-1H-pyrazolo[4,3-c]pyridin-7-yl)-2-oxo-2-[rac-(2S,4R,5S)-2-(4-fluorophenyl)-4-methoxy-5-methyl-1-piperidyl]acetamide (3.4 mg, 7.20 μmol, 34.00% yield, HCOOH)(RT=10.82 min) and N-(4-amino-1H-pyrazolo[4,3-c]pyridin-7-yl)-2-oxo-2-[rac-(2R,4S,5R)-2-(4-fluorophenyl)-4-methoxy-5-methyl-1-piperidyl]acetamide (3.1 mg, 6.56 μmol, 31.00% yield, HCOOH)(RT=16.97 min).

Rel Time for Compound 38 in analytical conditions (column: OJ-H, Hexane-IPA-MeOH, 50-25-25, 0.6 ml/min as mobile phase) 17.40 min and for Compound 7 10.96 min.

Compound 38: Retention time: 17.40 min. LCMS(ESI): [M+1]⁺ m/z: calcd 426.2; found 427.2; Rt=1.231 min.

Compound 7: Retention time: 10.96 min. LCMS(ESI): [M+1]⁺ m/z: calcd 426.2; found 427.2; Rt=1.230 min.

### Example 97. Synthesis of 2-oxo-N-(1H-pyrazolo[4,3-c]pyridin-7-yl)-2-[(2R,5S)-5-methyl-2-[2-(1-methyl-4-piperidyl) indazol-5-yl]-1-piperidyl]acetamide (Compound 84)

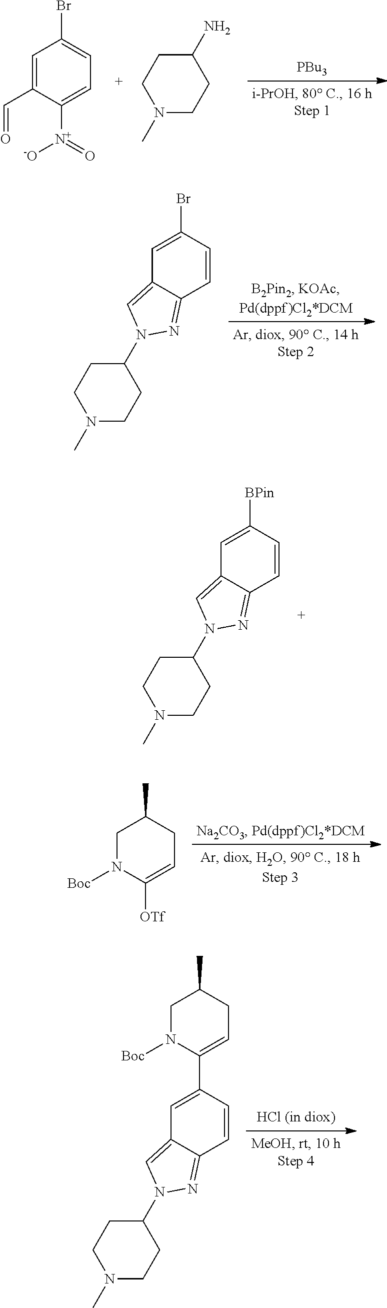

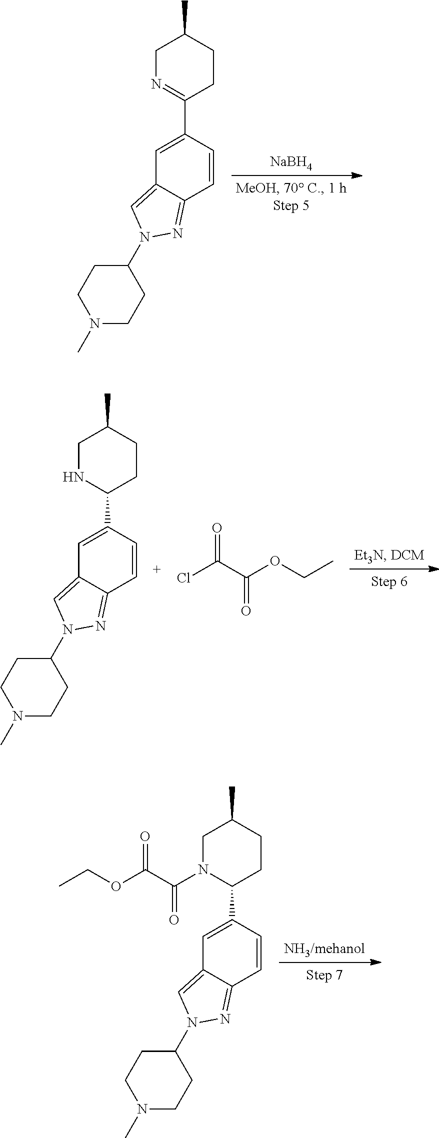

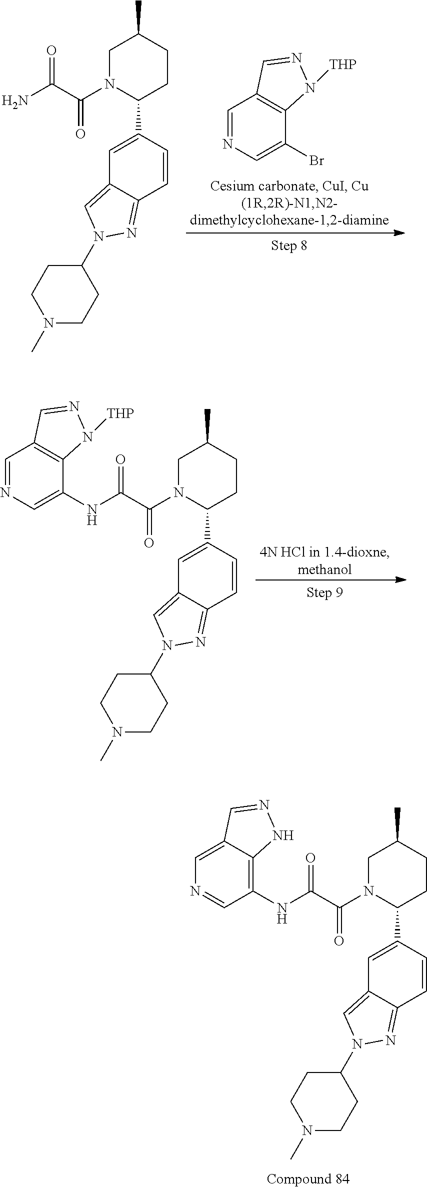

### Step 1: The Synthesis of 5-Bromo-2-(1-methyl-4-piperidyl)indazole

### Prepared by General Procedure Scheme S6 Step 1A

Yield: 20 g (78.19%); LCMS(ESI): [M+H]⁺ m/z: calcd 295.0; found 295.0; Rt=0.785 min.

### Step 2: The Synthesis of 2-(1-Methyl-4-piperidyl)-5-(4,4,5,5-tetramethyl-1,3,2-dioxaborolan-2-yl)indazole

### Prepared by General Procedure Scheme S6 Step 1

FCC conditions: Interchim; 330 g SiO₂, MTBE-MeOH from 0˜100%, flow rate=100 mL/min, cv=9.4

Yield: 9.0 g (77.59%); LCMS(ESI): [M+H]⁺ m/z: calcd 342.2; found 342.2; Rt=0.799 min.

### Step 3: The Synthesis of tert-Butyl (3S)-3-methyl-6-[2-(1-methyl-4-piperidyl) indazol-5-yl]-3,4-dihydro-2H-pyridine-1-carboxylate

### Prepared by General Procedure Scheme S6 Step 2

FCC conditions: Interchim; 120 g SiO₂, MTBE-MeOH from 0˜100%, flow rate=70 mL/min, cv=15.3

Yield: 6.0 g (55.41%); LCMS(ESI): [M+H]⁺ m/z: calcd 411.2; found 411.5; Rt=1.019 min.

### Step 4: The Synthesis of 2-(1-Methyl-4-piperidyl)-5-[(3S)-3-methyl-2,3,4,5-tetrahydropyridin-6-yl]indazole

### Prepared by General Procedure Scheme S6 Step 3 (Method B)

Yield: 1.2 g (79.35%); LCMS(ESI): [M+H]⁺ m/z: calcd 311.2; found 311.2; Rt=0.769 min.

### Step 5: The Synthesis of 2-(1-Methyl-4-piperidyl)-5-[(2R,5S)-5-methyl-2-piperidyl]indazole

### Prepared by General Procedure Scheme S6 Step 4

Yield: 3.2 g (79.48%); LCMS(ESI): [M+H]⁺ m/z: calcd 313.2; found 313.2; Rt=0.664 min.

### Step 6: The Synthesis of ethyl 2-oxo-2-[(2R,5S)-5-methyl-2-[2-(1-methyl-4-piperidyl) indazol-5-yl]-1-piperidyl]acetate

2-(1-methyl-4-piperidyl)-5-[(2R,5S)-5-methyl-2-piperidyl]indazole (1.50 g, 4.32 mmol) and TEA (437.21 mg, 4.32 mmol, 602.22 μL) was dissolved in DCM (40.04 mL), cooled with ice-water bath and then ethyl 2-chloro-2-oxo-acetate (619.41 mg, 4.54 mmol, 507.72 μL) in 20 ml of DCM was added dropwise in 10 min. Solution was stirred overnight. Reaction mixture was washed with aqueous solution of NaHCO₃, dried over sodium sulphate and evaporated to give ethyl 2-oxo-2-[(2R,5S)-5-methyl-2-[2-(1-methyl-4-piperidyl) indazol-5-yl]-1-piperidyl]acetate (1.1 g, 2.67 mmol, 61.72% yield)

LCMS(ESI): [M+H]⁺ m/z: calcd 412.5; found 413.2; Rt=1.044 min.

### Step 7: The Synthesis of 2-oxo-2-[(2R,5S)-5-methyl-2-[2-(1-methyl-4-piperidyl) indazol-5-yl]-1-piperidyl]acetamide

ethyl 2-oxo-2-[(2R,5S)-5-methyl-2-[2-(1-methyl-4-piperidyl) indazol-5-yl]-1-piperidyl]acetate (1.1 g, 2.67 mmol) was dissolved in NH₃/methanol (30 mL) solution (10% by weight) and stirred overnight, then concentrated in vacuo to give 2-oxo-2-[(2R,5S)-5-methyl-2-[2-(1-methyl-4-piperidyl) indazol-5-yl]-1-piperidyl]acetamide (0.9 g, 2.35 mmol, 88.01% yield).

LCMS(ESI): [M+H]⁺ m/z: calcd 383.5; found 384.0; Rt=0.826 min.

### Step 8: The Synthesis of 2-oxo-2-[(2R,5S)-5-methyl-2-[2-(1-methyl-4-piperidyl) indazol-5-yl]-1-piperidyl]-N-(1-tetrahydropyran-2-ylpyrazolo[4,3-c]pyridin-7-yl)acetamide

7-bromo-1-tetrahydropyran-2-yl-pyrazolo[4,3-c]pyridine (183.93 mg, 651.91 μmol), 2-oxo-2-[(2R,5S)-5-methyl-2-[2-(1-methyl-4-piperidyl) indazol-5-yl]-1-piperidyl]acetamide (0.25 g, 651.91 μmol), Copper (I) iodide (124.16 mg, 651.91 μmol, 22.09 μL), Cesium carbonate (424.81 mg, 1.30 mmol) and (1R,2R)—N1,N2-dimethylcyclohexane-1,2-diamine (139.09 mg, 977.87 μmol) were mixed in Dioxane (7.99 mL) under argon, and then stirred overnight at 100° C. for 12 hr in vial. Reaction mixture was filtered, then evaporated.

LCMS(ESI): [M+H]⁺ m/z: calcd 584.3; found 585.4; Rt=0.931 min.

### Step 9: The Synthesis of 2-oxo-N-(1H-pyrazolo[4,3-c]pyridin-7-yl)-2-[(2R,5S)-5-methyl-2-[2-(1-methyl-4-piperidyl) indazol-5-yl]-1-piperidyl]acetamide (Compound 84)

To a solution of 2-oxo-2-[(2R,5S)-5-methyl-2-[2-(1-methyl-4-piperidyl) indazol-5-yl]-1-piperidyl]-N-(1-tetrahydropyran-2-ylpyrazolo[4,3-c]pyridin-7-yl)acetamide (0.7 g, 1.20 mmol) in methanol (3 mL) was added HCl in dioxane (11.97 mmol) at 21° C. The resulting mixture was left to stirred for 3 hr. The resulting mixture was evaporated to dryness and subjected to HPLC (Device (Mobile Phase, Column): SYSTEM 2-10 min 50-100% methanol+nh3 flow 30 ml/min (loading pump 4 ml methanol), column: sun fire C18). After purification was obtained 2-oxo-N-(1H-pyrazolo[4,3-c]pyridin-7-yl)-2-[(2R,5S)-5-methyl-2-[2-(1-methyl-4-piperidyl) indazol-5-yl]-1-piperidyl]acetamide (0.033 g, 65.92 μmol, 5.51% yield).

¹H NMR (600 MHz, dmso) δ 1.04-1.10 (m, 3H), 1.33-1.43 (m, 1H), 1.69-1.96 (m, 3H), 2.07-2.22 (m, 7H), 2.26-2.30 (m, 1H), 3.05-3.08 (m, 2H), 3.72-4.11 (m, 1H), 4.49-4.55 (m, 1H), 5.46-5.83 (m, 1H), 7.01-7.60 (m, 2H), 7.61-7.68 (m, 2H), 8.06-8.22 (m, 1H), 8.26-8.44 (m, 3H), 8.44-8.63 (m, 1H), 8.85-9.01 (m, 1H), 11.05-11.21 (m, 1H), 13.00-13.19 (m, 1H).

LCMS(ESI): [M+H]⁺ m/z: calcd 500.6; found 501.0; Rt=1.377 min.

### Example 98. Synthesis of 2-(2-(3-(2-(dimethylamino)ethoxy)phenyl)-5-methylpiperidin-1-yl)-2-oxo-N-(1H-pyrazolo[4,3-c]pyridin-7-yl)acetamide (Compound 78 and Compound 114)

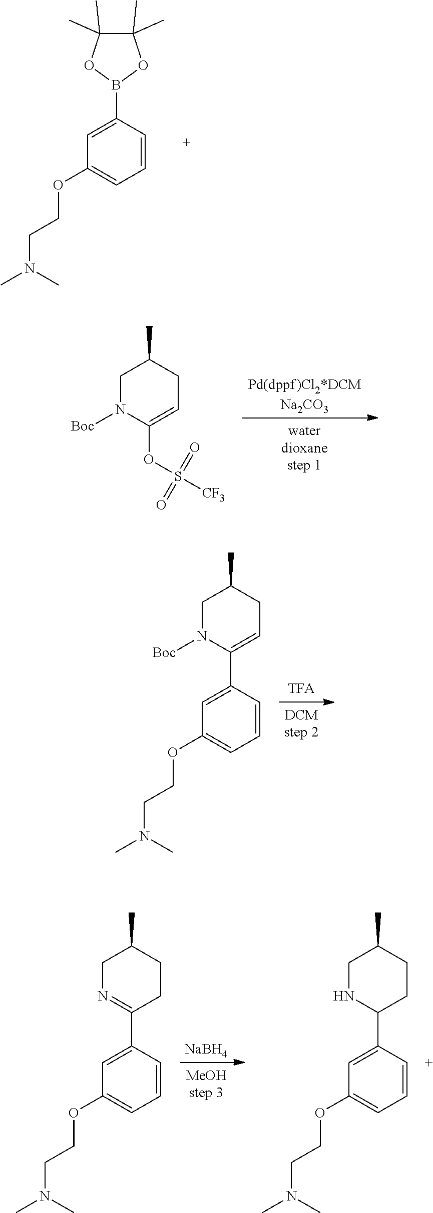

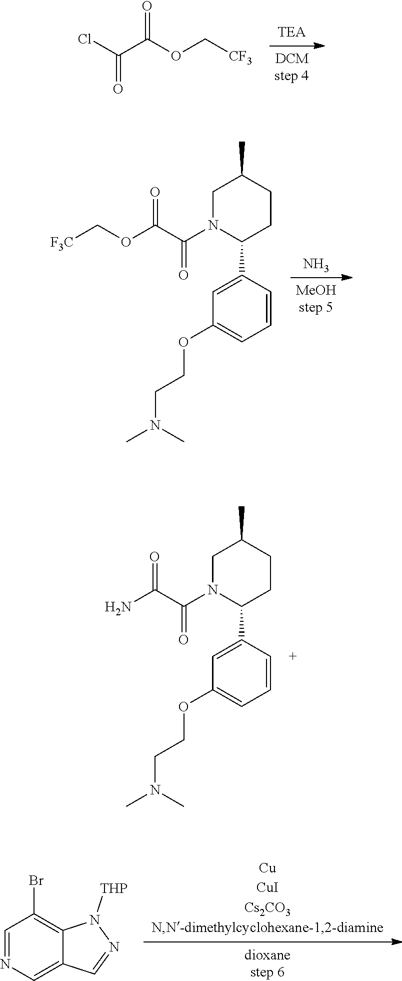

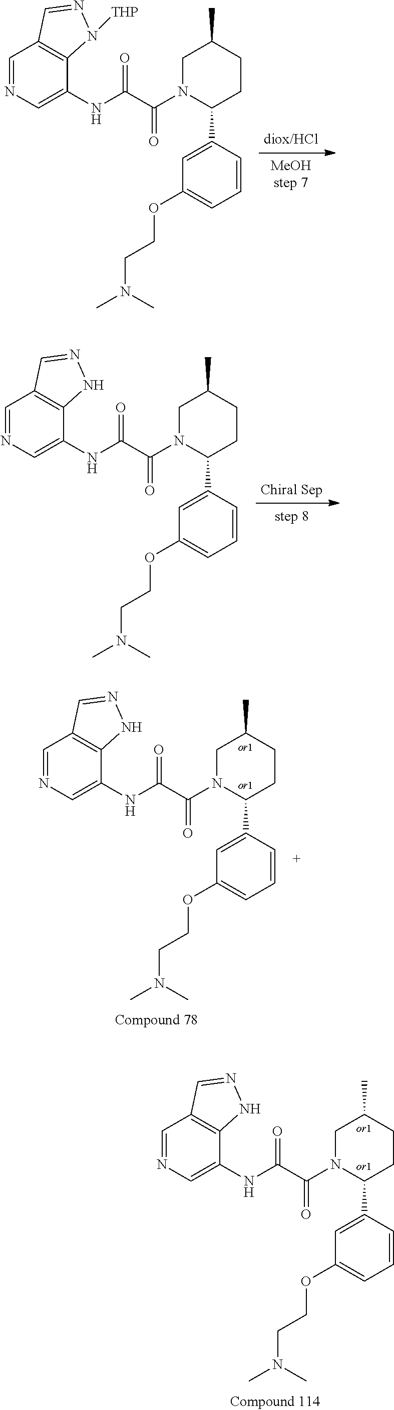

### Step 1: Synthesis of(S)-tert-butyl 6-(3-(2-(dimethylamino)ethoxy)phenyl)-3-methyl-3,4-dihydropyridine-1(2H)-carboxylate

tert-Butyl (3S)-3-methyl-6-(trifluoromethylsulfonyloxy)-3,4-dihydro-2H-pyridine-1-carboxylate (8 g, 23.17 mmol), N,N-dimethyl-2-[3-(4,4,5,5-tetramethyl-1,3,2-dioxaborolan-2-yl)phenoxy]ethanamine (5.62 g, 19.30 mmol) and sodium carbonate (4.09 g, 38.61 mmol, 1.62 mL) were mixed in a mixture of dioxane (45 mL) and water (15 mL). The resulting mixture was evacuated and backfilled three times with argon. Pd(dppf)Cl₂*DCM (788.26 mg, 965.25 μmol) was added to the previous mixture and the resulting mixture was heated at 90° C. overnight. The resulting mixture was cooled and diluted with water (100 ml). The resulting mixture was extracted with EtOAc (2*100 ml) and combined organic layers were washed with brine (60 ml), dried over Na₂SO₄, filtered, and concentrated in vacuum. The residue (10.8 g of crude product) was purified by column chromatography to obtain tert-butyl (3S)-6-[3-[2-(dimethylamino)ethoxy]phenyl]-3-methyl-3,4-dihydro-2H-pyridine-1-carboxylate (4.5 g, 12.48 mmol, 64.66% yield). LCMS(ESI): [M]⁺ m/z: calcd 360.2; found 361.2; Rt=1.077 min.

### Step 2: Synthesis of(S)—N,N-dimethyl-2-(3-(5-methyl-3,4,5,6-tetrahydropyridin-2-yl)phenoxy)ethanamine

tert-Butyl (3S)-6-[3-[2-(dimethylamino)ethoxy]phenyl]-3-methyl-3,4-dihydro-2H-pyridine-1-carboxylate (4.5 g, 12.48 mmol) was dissolved in DCM (18 mL) and TFA (18 mL) was added thereto. The resulting mixture was stirred for 1 hr. The reaction mixture was carefully poured into aq. NaHCO₃ solution (20 g in 100 ml of water) and the resulting mixture was extracted with DCM (2*50 ml). Combined organic layers were dried over sodium sulfate, filtered, and concentrated in vacuum to obtain N,N-dimethyl-2-[3-[(3S)-3-methyl-2,3,4,5-tetrahydropyridin-6-yl]phenoxy]ethanamine (2.36 g, 9.04 mmol, 72.46% yield). LCMS(ESI): [M]⁺ m/z: calcd 260.2; found 261.2; Rt=0.245 min.

### Step 3: Synthesis of N,N-dimethyl-2-(3-((5S)-5-methylpiperidin-2-yl)phenoxy)ethanamine

N,N-Dimethyl-2-[3-[(3S)-3-methyl-2,3,4,5-tetrahydropyridin-6-yl]phenoxy]ethanamine (2.36 g, 9.06 mmol) was dissolved in MeOH (45 mL) and sodium borohydride (685.82 mg, 18.13 mmol, 638.57 μL) was added portion wise. The resulting mixture was stirred overnight. Water (20 ml) was added to the reaction mixture and the resulting mixture was concentrated in vacuum. The residue was diluted with water (30 ml) and the resulting mixture was extracted with DCM (2*50 ml). Combined organic layers were dried over Na₂SO₄, filtered, and concentrated in vacuum to obtain N,N-dimethyl-2-[3-[(2R,5S)-5-methyl-2-piperidyl]phenoxy]ethanamine (2.02 g, 7.70 mmol, 84.94% yield). LCMS(ESI): [M]⁺ m/z: calcd 262.2; found 263.2; Rt=0.498 min.

### Step 4: Synthesis of 2,2,2-trifluoroethyl 2-((2R,5S)-2-(3-(2-(dimethylamino)ethoxy)phenyl)-5-methylpiperidin-1-yl)-2-oxoacetate

N,N-Dimethyl-2-[3-[(2R,5S)-5-methyl-2-piperidyl]phenoxy]ethanamine (1.02 g, 3.89 mmol) and TEA (433.55 mg, 4.28 mmol, 597.17 μL) were dissolved in DCM (25 mL) and the resulting mixture was cooled to −5° C. in an ice/MeOH bath. A solution of 2,2,2-trifluoroethyl 2-chloro-2-oxo-acetate (816.21 mg, 4.28 mmol) in DCM (5 mL) were added dropwise at −5° C. After addition completed, the resulting mixture was allowed to warm to rt and stirred overnight. The reaction mixture was transferred to a separation funnel and washed with aq.NaHCO₃ solution (30 ml) and water (30 ml). The organic layer was dried over Na₂SO₄, filtered, and concentrated in vacuum to obtain 2,2,2-trifluoroethyl 2-oxo-2-[(2R,5S)-2-[3-[2-(dimethylamino)ethoxy]phenyl]-5-methyl-1-piperidyl]acetate (1.28 g, 3.07 mmol, 78.92% yield). LCMS(ESI): [M]⁺ m/z: calcd 416.2; found 417.2; Rt=0.914 min.

### Step 5: Synthesis of 2-((2R,5S)-2-(3-(2-(dimethylamino)ethoxy)phenyl)-5-methylpiperidin-1-yl)-2-oxoacetamide

2,2,2-Trifluoroethyl 2-oxo-2-[(2R,5S)-2-[3-[2-(dimethylamino)ethoxy]phenyl]-5-methyl-1-piperidyl]acetate (1.28 g, 3.07 mmol) was dissolved in NH₃/MeOH (40 mL) and the resulting mixture was stirred overnight. The reaction mixture was concentrated in vacuum to obtain 2-oxo-2-[(2R,5S)-2-[3-[2-(dimethylamino)ethoxy]phenyl]-5-methyl-1-piperidyl]acetamide (890 mg, 2.67 mmol, 86.84% yield). LCMS(ESI): [M]⁺ m/z: calcd 333.2; found 334.2; Rt=0.828 min.

### Step 6: Synthesis of 2-((2R,5S)-2-(3-(2-(dimethylamino)ethoxy)phenyl)-5-methylpiperidin-1-yl)-2-oxo-N-(1-(tetrahydro-2H-pyran-2-yl)-1H-pyrazolo[4,3-c]pyridin-7-yl)acetamide

To an 8 ml vial 2-oxo-2-[(2R,5S)-2-[3-[2-(dimethylamino)ethoxy]phenyl]-5-methyl-1-piperidyl]acetamide (150 mg, 449.88 μmol), 7-bromo-1-tetrahydropyran-2-yl-pyrazolo[4,3-c]pyridine (126.93 mg, 449.88 μmol), copper (I) iodide (85.68 mg, 449.88 μmol, 15.25 μL), cesium carbonate (293.16 mg, 899.75 μmol), (1R,2R)—N1,N2-dimethylcyclohexane-1,2-diamine (95.99 mg, 674.82 μmol), copper (28.59 mg, 449.88 μmol) and dioxane (5.98 mL) were charged. The resulting mixture was purged with argon for 10 min and the vial was sealed and heated at 100° C. overnight. The reaction mixture was cooled, filtered, and concentrated in vacuum. The residue was purified by HPLC to obtain 2-oxo-2-[(2R,5S)-2-[3-[2-(dimethylamino)ethoxy]phenyl]-5-methyl-1-piperidyl]-N-(1-tetrahydropyran-2-ylpyrazolo[4,3-c]pyridin-7-yl)acetamide (43 mg, 80.43 μmol, 17.88% yield). LCMS(ESI): [M]⁺m/z: calcd 534.2; found 535.2; Rt=0.766 min.

### Step 7: Synthesis of 2-((2R,5S)-2-(3-(2-(dimethylamino)ethoxy)phenyl)-5-methylpiperidin-1-yl)-2-oxo-N-(1H-pyrazolo[4,3-c]pyridin-7-yl)acetamide

2-Oxo-2-[(2R,5S)-2-[3-[2-(dimethylamino)ethoxy]phenyl]-5-methyl-1-piperidyl]-N-(1-tetrahydropyran-2-ylpyrazolo[4,3-c]pyridin-7-yl)acetamide (43 mg, 80.43 μmol) was dissolved in dioxane (2 mL) and HCl/dioxane (3 mL) was added thereto. The resulting mixture was stirred overnight. The reaction mixture was concentrated in vacuum to obtain 2-oxo-N-(1H-pyrazolo[4,3-c]pyridin-7-yl)-2-[(2R,5S)-2-[3-[2-(dimethylamino)ethoxy]phenyl]-5-methyl-1-piperidyl]acetamide (43.9 mg, crude). LCMS(ESI): [M]⁺ m/z: calcd 450.2; found 451.2; Rt=0.725 min.

### Step 8: Chiral Separation (Compound 78 and Compound 114)

Racemic 2-oxo-N-(1H-pyrazolo[4,3-c]pyridin-7-yl)-2-[(2R,5S)-2-[3-[2-(dimethylamino)ethoxy]phenyl]-5-methyl-1-piperidyl]acetamide (43 mg, 95.44 μmol) was chiral separated (Column: Chiralcel OD-H (250*20 mm, 5 mkm); Mobile phase: Hexane-IPA-MeOH, 50-25-25. Flow Rate: 12 mL/min) to obtain 2-oxo-N-(1H-pyrazolo[4,3-c]pyridin-7-yl)-2-[(2R,5R)-2-[3-[2-(dimethylamino)ethoxy]phenyl]-5-methyl-1-piperidyl]acetamide (9.67 mg, 21.46 μmol, 22.49% yield (RT=10.62 min) and 2-oxo-N-(1H-pyrazolo[4,3-c]pyridin-7-yl)-2-[(2R,5S)-2-[3-[2-(dimethylamino)ethoxy]phenyl]-5-methyl-1-piperidyl]acetamide (9.72 mg, 21.57 μmol, 22.60% yield) (RT=16.1 min).

Rel Time for Compound 78 in analytical conditions (column: OD-H, Hexane-IPA-MeOH, 50-25-25, 0.6 ml/min as mobile phase) 16.38 min and for Compound 114 10.76 min.

Compound 78: Retention time: 16.38 min. ¹H NMR (600 MHZ, DMSO-d₆) δ (ppm) 1.01-1.08 (m, 3H), 1.29-1.41 (m, 1H), 1.64-1.77 (m, 1H), 1.84-1.95 (m, 1H), 1.99-2.12 (m, 1H), 2.16-2.22 (m, 1H), 2.22-2.32 (m, 6H), 2.63-2.79 (m, 2H), 2.81-3.19 (m, 1H), 3.71-4.02 (m, 1H), 4.03-4.13 (m, 2H), 5.34-5.71 (m, 1H), 6.80-6.90 (m, 2H), 6.90-6.97 (m, 1H), 7.25-7.38 (m, 1H), 8.27-8.39 (m, 1H), 8.41-8.57 (m, 1H), 8.86-9.02 (m, 1H), 11.10 (d, 1H), 13.07 (s, 1H). LCMS(ESI): [M]⁺ m/z: calcd 450.2; found 451.2; Rt=1.660 min.

Compound 114: Retention time: 10.76 min. ¹H NMR (600 MHz, DMSO-d₆) δ (ppm) 0.72-0.84 (m, 3H), 1.03-1.14 (m, 1H), 1.60-1.87 (m, 3H), 1.88-2.02 (m, 1H), 2.22-2.32 (m, 6H), 2.61-2.84 (m, 3H), 3.89-4.04 (m, 1H), 4.05-4.33 (m, 2H), 5.40-5.72 (m, 1H), 6.82-6.86 (m, 1H), 6.86-6.90 (m, 1H), 6.90-6.97 (m, 1H), 7.28-7.36 (m, 1H), 8.30-8.41 (m, 1H), 8.43-8.58 (m, 1H), 8.88-9.00 (m, 1H), 11.07-11.20 (m, 1H), 12.99-13.25 (m, 1H). LCMS(ESI): [M]⁺ m/z: calcd 450.2; found 451.2; Rt=0.813 min.

### Example 99. Synthesis of 2-((2R,5S)-5-methyl-2-(2-(1-methylpiperidin-4-yl) quinolin-7-yl)piperidin-1-yl)-2-oxo-N-(1H-pyrazolo[4,3-c]pyridin-7-yl)acetamide (Compound 42)

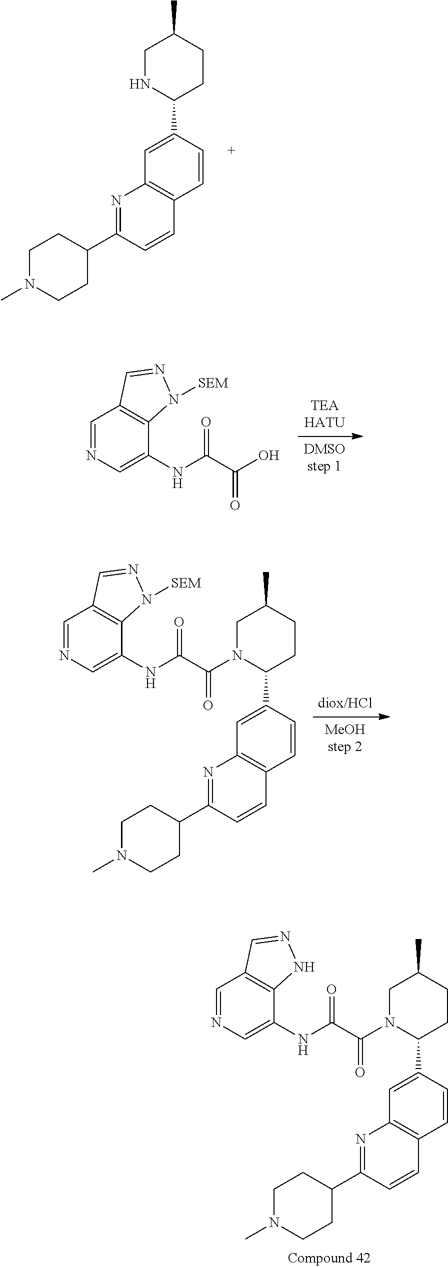 [SMILES: C[C@H]1CC[C@H](c2ccc3ccc(C4CCN(C)CC4)nc3c2)NC1 ; C[Si](C)(C)CCOCn1ncc2cncc(NC(=O)C(=O)O)c21 ; C[C@H]1CC[C@H](c2ccc3ccc(C4CCN(C)CC4)nc3c2)N(C(=O)C(=O)Nc2cncc3cnn(COCC[Si](C)(C)C)c23)C1 ; C[C@H]1CC[C@H](c2ccc3ccc(C4CCN(C)CC4)nc3c2)N(C(=O)C(=O)Nc2cncc3cn[nH]c23)C1]

The synthesis of 2-(1-methyl-4-piperidyl)-7-[(2R,5S)-5-methyl-2-piperidyl]quinoline is given in Intermediate 22

### Step 1: Synthesis of 2-((2R,5S)-5-methyl-2-(2-(1-methylpiperidin-4-yl) quinolin-7-yl)piperidin-1-yl)-2-oxo-N-(1-((2-(trimethylsilyl)ethoxy)methyl)-1H-pyrazolo[4,3-c]pyridin-7-yl)acetamide

Crude product 2-(1-methyl-4-piperidyl)-7-[(2R,5S)-5-methyl-2-piperidyl]quinoline (100.00 mg, 216.40 μmol) from previous stage was mixed with TEA (109.49 mg, 1.08 mmol, 150.81 μL) in DMSO (2 mL), next 2-oxo-2-[[1-(2-trimethylsilylethoxymethyl)pyrazolo[4,3-c]pyridin-7-yl]amino]acetic acid (Intermediate 37)(109.20 mg, 324.60 μmol) and HATU (123.42 mg, 324.60 μmol) were added and stirred overnight. RM was treated with water and desired product was filtered, washed with water and dissolved in MeOH, then subjected to HPLC (SYSTEM 2-10 min 10-40% MeOH+NH₃ 30 ml/min (loading pump 4 m MeOH) column: SunFire 100*19 mm, 5 microM). 2-Oxo-2-[(2R,5S)-5-methyl-2-[2-(1-methyl-4-piperidyl)-7-quinolyl]-1-piperidyl]-N-[1-(2-trimethylsilylethoxymethyl)pyrazolo[4,3-c]pyridin-7-yl]acetamide (47 mg, 73.22 μmol, 33.84% yield) was obtained. LCMS(ESI): [M]⁺ m/z: calcd 641.2; found 642.2; Rt=1.144 min.

### Step 2: Synthesis of 2-((2R,5S)-5-methyl-2-(2-(1-methylpiperidin-4-yl) quinolin-7-yl)piperidin-1-yl)-2-oxo-N-(1H-pyrazolo[4,3-c]pyridin-7-yl)acetamide (Compound 42)

2-Oxo-2-[(2R,5S)-5-methyl-2-[2-(1-methyl-4-piperidyl)-7-quinolyl]-1-piperidyl]-N-[1-(2-trimethylsilylethoxymethyl)pyrazolo[4,3-c]pyridin-7-yl]acetamide (47 mg, 65.90 μmol) was dissolved in methanol (1 mL), next 3 ml of HCl/diox (65.90 μmol, 3 mL)(10% of HCl by weight) was added and stirred overnight. The RM was concentrated in vacuum, then dissolved in 1 ml of DMSO and subjected to HPLC (2-10 min 25-50% water MeOH+NH₃ 30/min; loading pump 4 ml/min MeOH+NH₃ column SunFire 19*100 mm). 2-Oxo-N-(1H-pyrazolo[4,3-c]pyridin-7-yl)-2-[(2R,5S)-5-methyl-2-[2-(1-methyl-4-piperidyl)-7-quinolyl]-1-piperidyl]acetamide (12.3 mg, 24.04 μmol, 36.48% yield) was obtained.

Compound 42: ¹H NMR (600 MHZ, DMSO-d₆) δ (ppm) 1.01-1.15 (m, 3H), 1.35-1.51 (m, 1H), 1.74-1.82 (m, 1H), 1.85-1.91 (m, 4H), 1.91-2.01 (m, 1H), 2.08-2.14 (m, 2H), 2.14-2.22 (m, 1H), 2.26 (s, 3H), 2.27-2.36 (m, 1H), 2.80-2.85 (m, 1H), 2.92-2.96 (m, 2H), 3.90-4.14 (m, 1H), 5.74 (d, 1H), 7.38-7.49 (m, 1H), 7.51-7.65 (m, 1H), 7.87 (s, 1H), 7.89-8.02 (m, 1H), 8.17-8.24 (m, 1H), 8.24-8.40 (m, 2H), 8.41-8.76 (m, 1H), 8.83-9.19 (m, 1H), 10.95-11.54 (m, 1H), 13.01-13.54 (m, 1H). LCMS(ESI): [M]⁺ m/z: calcd 511.2; found 512.2; Rt=2.069 min.

### Example 100. Synthesis of N-(4-amino-1H-pyrazolo[4,3-c]pyridin-7-yl)-2-((2R,5S)-5-methyl-2-(3,4,5-trifluorophenyl)piperidin-1-yl)-2-oxoacetamide (Compound 18)

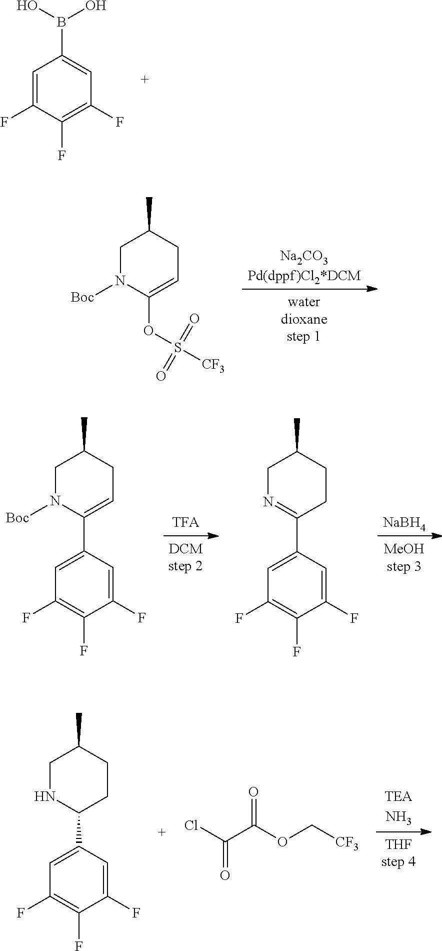

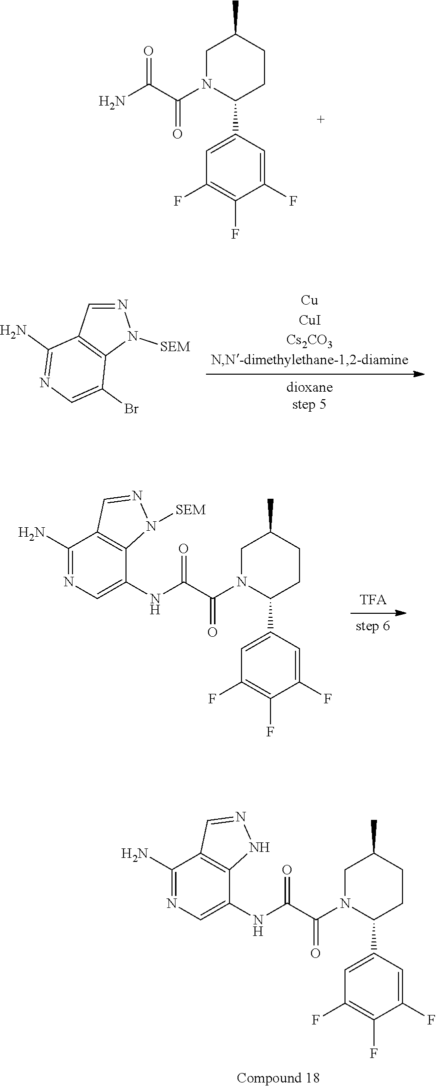 [SMILES: C[C@H]1CC[C@H](c2cc(F)c(F)c(F)c2)N(C(=O)C(N)=O)C1 ; C[Si](C)(C)CCOCn1ncc2c(N)ncc(Br)c21 ; C[C@H]1CC[C@H](c2cc(F)c(F)c(F)c2)N(C(=O)C(=O)Nc2cnc(N)c3cnn(COCC[Si](C)(C)C)c23)C1 ; C[C@H]1CC[C@H](c2cc(F)c(F)c(F)c2)N(C(=O)C(=O)Nc2cnc(N)c3cn[nH]c23)C1]

### Step 1: Synthesis of(S)-tert-butyl 3-methyl-6-(3,4,5-trifluorophenyl)-3,4-dihydropyridine-1(2H)-carboxylate

tert-Butyl (3S)-3-methyl-6-(trifluoromethylsulfonyloxy)-3,4-dihydro-2H-pyridine-1-carboxylate (5.01 g, 14.50 mmol), sodium carbonate (3.62 g, 34.11 mmol, 1.43 mL) and water (20 mL) were added to a solution (3,4,5-trifluorophenyl) boronic acid (3 g, 17.06 mmol) in dioxane (100 mL). The resulting mixture was evacuated and then backfilled with argon. This operation was repeated two times, then Pd(dppf)Cl₂*DCM (696.39 mg, 852.75 μmol) was added and the reaction mixture was stirred under argon at 85° C. for 12 hr, then cooled down and concentrated in vacuum. The residue was diluted with MTBE (120 ml) and stirred for 0.5 hr. After the most of the residue had dissolved, anhydrous sodium sulfate was added, and the resulting mixture was filtered. The filter cake was additionally washed with MTBE (3*20 ml) and discarded. The filtrate was concentrated in vacuum to afford crude tert-butyl (3S)-3-methyl-6-(3,4,5-trifluorophenyl)-3,4-dihydro-2H-pyridine-1-carboxylate (6 g, crude) as brown gum, which was used directly in the next step. LCMS(ESI): [M]⁺ m/z: calcd 327.2; found 328.2; Rt=1.662 min.

### Step 2: Synthesis of(S)-3-methyl-6-(3,4,5-trifluorophenyl)-2,3,4,5-tetrahydropyridine

TFA (37.00 g, 324.50 mmol, 25 mL) was added in one portion to a stirred solution of tert-butyl (3S)-3-methyl-6-(3,4,5-trifluorophenyl)-3,4-dihydro-2H-pyridine-1-carboxylate (6 g, 18.33 mmol) in DCM (30 mL). The resulting solution was stirred at 25° C. for 0.5 hr, and then concentrated in vacuum. The residue was diluted with water (100 ml). The resulting solution of TFA salt of the product was decanted from dark-brown oily residue, which was additionally rinsed with water (2*20 ml). The combined aqueous solution was filtered through a cotton pad to remove traces of oily impurities, then basified to pH 11-12 with 10% aqueous sodium carbonate solution and extracted with DCM (2*40 ml). The combined organic extracts were dried over sodium sulfate and concentrated in vacuum to afford (3S)-3-methyl-6-(3,4,5-trifluorophenyl)-2,3,4,5-tetrahydropyridine (1.4 g, 6.16 mmol, 33.61% yield) as brown gum, which was directly used in the next step. LCMS(ESI): [M]⁺ m/z: calcd 227.2; found 228.2; Rt=0.887 min.

### Step 3: Synthesis of (2R,5S)-5-methyl-2-(3,4,5-trifluorophenyl)piperidine

Sodium borohydride (699.24 mg, 18.48 mmol, 651.07 μL) was added in one portion at 0° C. to a stirred solution of (3S)-3-methyl-6-(3,4,5-trifluorophenyl)-2,3,4,5-tetrahydropyridine (1.4 g, 6.16 mmol) in MeOH (9.47 mL). The reaction mixture was stirred at 0° C. for 1 hr, and then concentrated in vacuum. The residue was diluted with water (10 ml) and extracted with DCM (2*15 ml). The combined organic extracts were dried over sodium sulfate and concentrated in vacuum to afford crude (2R,5S)-5-methyl-2-(3,4,5-trifluorophenyl)piperidine (1.1 g, 4.80 mmol, 77.88% yield) as yellow gum, which was used directly in the next step.

LCMS(ESI): [M]⁺ m/z: calcd 229.2; found 230.2; Rt=0.900 min.

### Step 4: Synthesis of 2-((2R,5S)-5-methyl-2-(3,4,5-trifluorophenyl)piperidin-1-yl)-2-oxoacetamide

2,2,2-Trifluoroethyl 2-chloro-2-oxo-acetate (432.13 mg, 2.27 mmol) was added slowly to a cooled to −10° C. mixture of (2R,5S)-5-methyl-2-(3,4,5-trifluorophenyl)piperidine (400 mg, 1.74 mmol) and TEA (882.83 mg, 8.72 mmol, 1.22 mL) in THF. The resulting mixture was allowed to warm to 25° C. and stirred for 2 hr. Then gaseous ammonia (29.72 mg, 1.74 mmol) was vigorously bubbled through it at 25° C. for 1 hr. The resulting mixture was filtered to remove ammonium chloride, the filter cake was washed with THF (3*15 ml) and discarded. The combined filtrate was concentrated in vacuum to afford crude 2-oxo-2-[(2R,5S)-5-methyl-2-(3,4,5-trifluorophenyl)-1-piperidyl]acetamide (600 mg, crude) as yellow gum, which was used directly in the next step. LCMS(ESI): [M]⁺ m/z: calcd 300.2; found 301.2; Rt=1.267 min.

### Step 5: Synthesis of N-(4-amino-1-((2-(trimethylsilyl)ethoxy)methyl)-1H-pyrazolo[4,3-c]pyridin-7-yl)-2-((2R,5S)-5-methyl-2-(3,4,5-trifluorophenyl)piperidin-1-yl)-2-oxoacetamide

A mixture of 2-oxo-2-[(2R,5S)-5-methyl-2-(3,4,5-trifluorophenyl)-1-piperidyl]acetamide (350 mg, 1.17 mmol), 7-bromo-1-(2-trimethylsilylethoxymethyl)pyrazolo[4,3-c]pyridin-4-amine (290.84 mg, 847.19 μmol), copper (10 mg, 157.36 μmol), copper (I) iodide (80 mg, 420.06 μmol, 14.23 μL), cesium carbonate (700 mg, 2.15 mmol) and N¹,N²-dimethylethane-1,2-diamine (110 mg, 773.34 μmol) in dioxane (6 mL) was stirred in a sealed vial under argon at 105° C. for 48 hr. The resulting mixture was cooled down and submitted to reverse phase HPLC (column: XBridge BEH C18 100×19 mm, 5 um; mobile phase: 50-80% 0-1-6 min H₂O/MeOH/0.1% NH₄OH, flow rate: 30 ml/min (loading pump 4 ml/min to MeOH) afford N-[4-amino-1-(2-trimethylsilylethoxymethyl)pyrazolo[4,3-c]pyridin-7-yl]-2-oxo-2-[(2R,5S)-5-methyl-2-(3,4,5-trifluorophenyl)-1-piperidyl]acetamide as light-brown gum, which was used directly in the next step.

### Step 6: Synthesis of N-(4-amino-1H-pyrazolo[4,3-c]pyridin-7-yl)-2-((2R,5S)-5-methyl-2-(3,4,5-trifluorophenyl)piperidin-1-yl)-2-oxoacetamide (Compound 18)

A solution of N-[4-amino-1-(2-trimethylsilylethoxymethyl)pyrazolo[4,3-c]pyridin-7-yl]-2-oxo-2-[(2R,5S)-5-methyl-2-(3,4,5-trifluorophenyl)-1-piperidyl]acetamide (33 mg, 58.65 μmol) in TFA (1.48 g, 12.98 mmol, 1 mL) was stirred at 25° C. for 3 hr. The solvent was evaporated and residue as purified by HPLC (Device (Mobile Phase, Column): SYSTEM 20-20-70% 0-1-6 min H₂O/MeOH/0.1% NH₄OH, flow: 30 ml/min (loading pump 4 ml/min MeOH) target mass 432 column: YMC Triart C18 100×20 mm, 5 um) to give N-(4-amino-1H-pyrazolo[4,3-c]pyridin-7-yl)-2-oxo-2-[(2R,5S)-5-methyl-2-(3,4,5-trifluorophenyl)-1-piperidyl]acetamide (4 mg, 9.25 μmol, 15.77% yield).

Compound 18: ¹H NMR (600 MHZ, DMSO-d₆) δ (ppm) 0.78-1.05 (m, 3H), 1.26-1.42 (m, 1H), 1.56-1.73 (m, 1H), 1.73-1.91 (m, 1H), 1.92-2.11 (m, 1H), 2.11-2.31 (m, 1H), 2.75-3.22 (m, 1H), 3.45-4.14 (m, 1H), 4.81-5.58 (m, 1H), 6.28-6.88 (m, 2H), 7.27 (d, 2H), 7.46-7.79 (m, 1H), 8.03-8.38 (m, 1H), 9.57-10.69 (m, 1H), 12.43-13.37 (m, 1H).

LCMS(ESI): [M]⁺ m/z: calcd 432.2; found 433.2; Rt=2.587 min.

### Example 101. The synthesis of N-(4-amino-1H-pyrazolo[4,3-c]pyridin-7-yl)-2-((2R,5S)-5-methyl-2-(3,4,5-trifluorophenyl)piperidin-1-yl)-2-oxoacetamide (Compound 18)

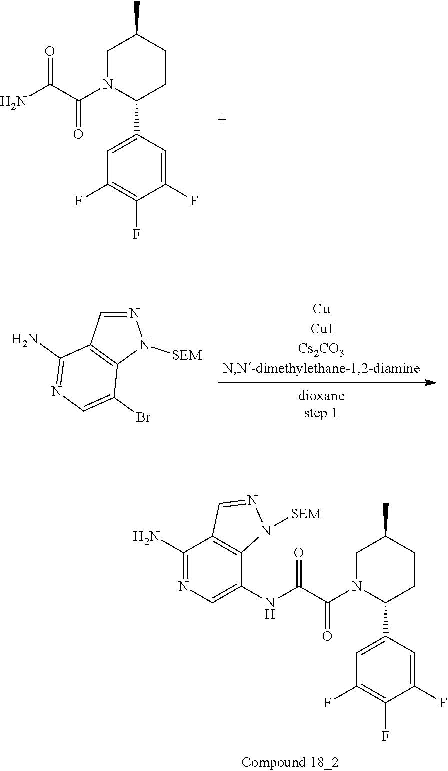 [SMILES: C[C@H]1CC[C@H](c2cc(F)c(F)c(F)c2)N(C(=O)C(N)=O)C1 ; C[Si](C)(C)CCOCn1ncc2c(N)ncc(Br)c21 ; C[C@H]1CC[C@H](c2cc(F)c(F)c(F)c2)N(C(=O)C(=O)Nc2cnc(N)c3cnn(COCC[Si](C)(C)C)c23)C1]

The synthesis of 2-((2R,5S)-5-methyl-2-(3,4,5-trifluorophenyl)piperidin-1-yl)-2-oxoacetamide is given in Example 100.

### Step 1: Synthesis of N-(4-amino-1-((2-(trimethylsilyl)ethoxy)methyl)-1H-pyrazolo[4,3-c]pyridin-7-yl)-2-((2R,5S)-5-methyl-2-(3,4,5-trifluorophenyl)piperidin-1-yl)-2-oxoacetamide

To a mixture of 2-oxo-2-[(2R,5S)-5-methyl-2-(3,4,5-trifluorophenyl)-1-piperidyl]acetamide (350 mg, 1.17 mmol), 7-bromo-1-(2-trimethylsilylethoxymethyl)pyrazolo[4,3-c]pyridin-4-amine (460.16 mg, 1.34 mmol), copper (14.81 mg, 233.12 μmol), copper (I) iodide (221.99 mg, 1.17 mmol, 39.50 μL), cesium carbonate (759.55 mg, 2.33 mmol) and N¹,N²-dimethylethane-1,2-diamine (248.69 mg, 1.75 mmol), dioxane (6 mL) was added. The resulting mixture was evacuated, refiled with Argon three time, heated at 100° C. for 18 hr and cooled. The inorganic precipitate was filtered and solution was subjected to HPLC (Device (Mobile Phase, Column): SYSTEM 70-80% 0-5 min H₂O/MeOH/0.1% NH₄OH, flow: 30 ml/min (loading pump 4 ml/min MeOH) target mass 563 column: YMC Triart C18 100×20 mm, 5 um) to give N-[4-amino-1-(2-trimethylsilylethoxymethyl)pyrazolo[4,3-c]pyridin-7-yl]-2-oxo-2-[(2R,5S)-5-methyl-2-(3,4,5-trifluorophenyl)-1-piperidyl]acetamide (232 mg, 412.33 μmol, 35.37% yield). LCMS(ESI): [M]⁺ m/z: calcd 562.2; found 563.2; Rt=1.115 min.

### Step 2: Synthesis of N-(4-amino-1H-pyrazolo[4,3-c]pyridin-7-yl)-2-((2R,5S)-5-methyl-2-(3,4,5-trifluorophenyl)piperidin-1-yl)-2-oxoacetamide (Compound 18)

A solution of N-[4-amino-1-(2-trimethylsilylethoxymethyl)pyrazolo[4,3-c]pyridin-7-yl]-2-oxo-2-[(2R,5S)-5-methyl-2-(3,4,5-trifluorophenyl)-1-piperidyl]acetamide (220 mg, 391.00 μmol) in TFA (2.96 g, 25.96 mmol, 2 mL) was stirred at 25° C. for 3 hr. The solvent was evaporated and residue was purified by HPLC (Device (Mobile Phase, Column): SYSTEM 50-50-70% 0-1.5-6 min H₂O/MeOH/0.1% NH₄OH, flow: 30 ml/min (loading pump 4 ml/min MeOH) target mass 433 column: YMC Triart C18 100×20 mm, 5 um) to give N-(4-amino-1H-pyrazolo[4,3-c]pyridin-7-yl)-2-oxo-2-[(2R,5S)-5-methyl-2-(3,4,5-trifluorophenyl)-1-piperidyl]acetamide (75 mg, 173.45 μmol, 44.36% yield).

Compound 18: ¹H NMR (600 MHz, DMSO-d₆) δ (ppm) 0.78-1.05 (m, 3H), 1.26-1.42 (m, 1H), 1.56-1.73 (m, 1H), 1.73-1.91 (m, 1H), 1.92-2.11 (m, 1H), 2.11-2.31 (m, 1H), 2.75-3.22 (m, 1H), 3.45-4.14 (m, 1H), 4.81-5.58 (m, 1H), 6.28-6.88 (m, 2H), 7.27 (d, 2H), 7.46-7.79 (m, 1H), 8.03-8.38 (m, 1H), 9.57-10.69 (m, 1H), 12.43-13.37 (m, 1H).

LCMS(ESI): [M]⁺ m/z: calcd 432.2; found 433.2; Rt=0.797 min.

### Example 102. Synthesis of N-(4-amino-1H-pyrazolo[4,3-c]pyridin-7-yl)-2-((2R,5S)-5-methyl-2-(quinolin-6-yl)piperidin-1-yl)-2-oxoacetamide (Compound 83)

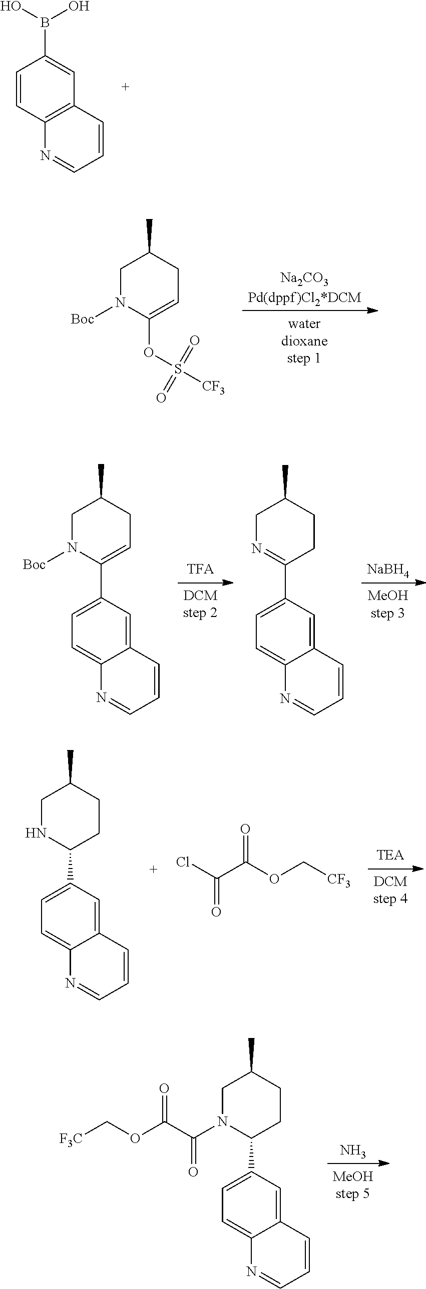

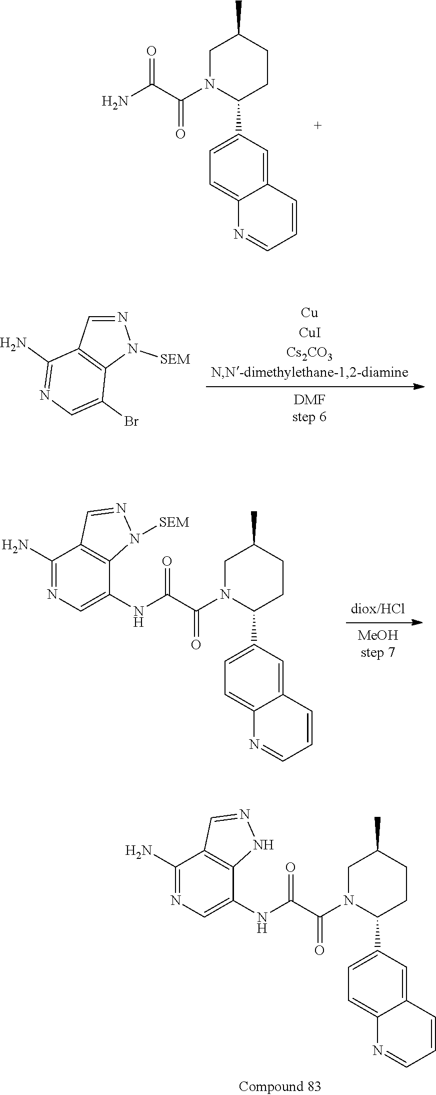 [SMILES: C[C@H]1CC[C@H](c2ccc3ncccc3c2)N(C(=O)C(N)=O)C1 ; C[Si](C)(C)CCOCn1ncc2c(N)ncc(Br)c21 ; C[C@H]1CC[C@H](c2ccc3ncccc3c2)N(C(=O)C(=O)Nc2cnc(N)c3cnn(COCC[Si](C)(C)C)c23)C1 ; C[C@H]1CC[C@H](c2ccc3ncccc3c2)N(C(=O)C(=O)Nc2cnc(N)c3cn[nH]c23)C1]

### Step 1: Synthesis of(S)-tert-butyl 3-methyl-6-(quinolin-6-yl)-3,4-dihydropyridine-1(2H)-carboxylate

tert-Butyl (3S)-3-methyl-6-(trifluoromethylsulfonyloxy)-3,4-dihydro-2H-pyridine-1-carboxylate (3.65 g, 10.58 mmol), 6-(4,4,5,5-tetramethyl-1,3,2-dioxaborolan-2-yl) quinoline (2.7 g, 10.58 mmol) and sodium carbonate (3.37 g, 31.75 mmol, 1.33 mL) was added to a mixture of dioxane (58.69 mL) and water (19.56 mL). The resulting mixture was evacuated and then backfilled with argon, this operation was repeated three times, then Pd(dppf)Cl₂*DCM (432.13 mg, 529.16 μmol) was added under argon. The reaction mixture was stirred under argon at 70° C. for 16 hr, then cooled and filtered. The filter cake was washed with dioxane (2*20 ml) and discarded. The filtrate was evaporated in vacuum to afford tert-butyl (3S)-3-methyl-6-(6-quinolyl)-3,4-dihydro-2H-pyridine-1-carboxylate (3.4 g, 10.48 mmol, 99.03% yield). LCMS(ESI): [M]⁺ m/z: calcd 324.2; found 325.2; Rt=1.205 min.

### Step 2: Synthesis of(S)-6-(5-methyl-3,4,5,6-tetrahydropyridin-2-yl) quinoline

The solution of tert-butyl (3S)-3-methyl-6-(6-quinolyl)-3,4-dihydro-2H-pyridine-1-carboxylate (3.4 g, 10.48 mmol) in TFA (17.93 g, 157.21 mmol, 12.11 mL) was stirred at 25° C. for 1 hr, and then evaporated in vacuum. Crushed ice (15 g) was added to the residue and filtered and pH was adjusted to 10 with a 10% aqueous solution of sodium hydroxide. The resulting mixture was extracted with DCM (2*30 ml). The combined organic extracts were dried over sodium sulphate and evaporated in vacuum to afford 6-[(3S)-3-methyl-2,3,4,5-tetrahydropyridin-6-yl]quinoline (1.7 g, 7.58 mmol, 72.32% yield) as brown oil, which was used directly in the next step. LCMS(ESI): [M]⁺ m/z: calcd 224.2; found 225.2; Rt=0.736 min.

### Step 3: Synthesis of 6-((2R,5S)-5-methylpiperidin-2-yl) quinoline

To a stirred solution of 6-[(3S)-3-methyl-2,3,4,5-tetrahydropyridin-6-yl]quinoline (1.7 g, 7.58 mmol) in MeOH (49.47 mL) was added sodium borohydride (573.48 mg, 15.16 mmol, 533.96 μL) at 0° C. The resulting reaction mixture was stirred at 25° C. for 15 hr. Upon completion, the reaction mixture was concentrated under reduced pressure, and then quenched with water 20 mL and 50 ml EtOAc. The combined organic phase was washed with Brine 20 mL, dried over Na₂SO₄ and concentrated under reduced pressure to obtain 6-[(2R,5S)-5-methyl-2-piperidyl]quinoline (1.3 g, 5.74 mmol, 75.79% yield), which was used in next step without farther purification. LCMS(ESI): [M]⁺ m/z: calcd 226.2; found 227.2; Rt=0.706 min.

### Step 4: Synthesis of 2,2,2-trifluoroethyl 2-((2R,5S)-5-methyl-2-(quinolin-6-yl)piperidin-1-yl)-2-oxoacetate

6-[(2R,5S)-5-Methyl-2-piperidyl]quinoline (1.3 g, 5.74 mmol) was dissolved in DCM (48.80 mL) and TEA (871.88 mg, 8.62 mmol, 1.20 mL) was added. Then reaction mixture was cooled and 2,2,2-trifluoroethyl 2-chloro-2-oxo-acetate (1.31 g, 6.89 mmol) was added dropwise. The reaction was stirred at rt overnight. After that water (50 ml) was added and organic layer was washed with brine (20 ml), dried over Na₂SO₄ and evaporated in vacuum to afford 2,2,2-trifluoroethyl 2-oxo-2-[(2R,5S)-5-methyl-2-(6-quinolyl)-1-piperidyl]acetate (1.6 g, 4.21 mmol, 73.23% yield). LCMS(ESI): [M]⁺ m/z: calcd 380.2; found 381.2; Rt=1.077 min.

### Step 5: Synthesis of 2-((2R,5S)-5-methyl-2-(quinolin-6-yl)piperidin-1-yl)-2-oxoacetamide

2,2,2-Trifluoroethyl 2-oxo-2-[(2R,5S)-5-methyl-2-(6-quinolyl)-1-piperidyl]acetate (1.6 g, 4.21 mmol) was dissolved in NH₃/MeOH and stirred overnight at rt. Then it was evaporated in vacuum to afford 2-oxo-2-[(2R,5S)-5-methyl-2-(6-quinolyl)-1-piperidyl]acetamide (1.2 g, 4.04 mmol, 95.94% yield). LCMS(ESI): [M]⁺ m/z: calcd 297.2; found 298.2; Rt=0.844 min.

### Step 6: Synthesis of N-(4-amino-1-((2-(trimethylsilyl)ethoxy)methyl)-1H-pyrazolo[4,3-c]pyridin-7-yl)-2-((2R,5S)-5-methyl-2-(quinolin-6-yl)piperidin-1-yl)-2-oxoacetamide

A mixture of 7-bromo-1-(2-trimethylsilylethoxymethyl)pyrazolo[4,3-c]pyridin-4-amine (115.45 mg, 336.30 μmol), 2-oxo-2-[(2R,5S)-5-methyl-2-(6-quinolyl)-1-piperidyl]acetamide (0.1 g, 336.30 μmol), copper (I) iodide (32.02 mg, 168.15 μmol, 5.70 μL), cesium carbonate (219.15 mg, 672.61 μmol) and N¹,N²-dimethylethane-1,2-diamine (47.84 mg, 336.30 μmol) in DMF (1.99 mL) was stirred in a sealed vial under argon at 110° C. for 16 hr. The resulting mixture was cooled down and submitted to reverse phase HPLC (column: SunFire C18 100×19 mm, 5 um; mobile phase: 25-50% 2-10 min water-MeOH, flow rate: 30 ml/min (loading pump 4 ml/min MeOH) to afford N-[4-amino-1-(2-trimethylsilylethoxymethyl)pyrazolo[4,3-c]pyridin-7-yl]-2-oxo-2-[(2R,5S)-5-methyl-2-(6-quinolyl)-1-piperidyl]acetamide (16.2 mg, 28.94 μmol, 8.61% yield), which was used directly in the next step. LCMS(ESI): [M]⁺ m/z: calcd 559.2; found 560.2; Rt=1.220 min.

### Step 7: Synthesis of N-(4-amino-1H-pyrazolo[4,3-c]pyridin-7-yl)-2-((2R,5S)-5-methyl-2-(quinolin-6-yl)piperidin-1-yl)-2-oxoacetamide (Compound 83)

N-[4-Amino-1-(2-trimethylsilylethoxymethyl)pyrazolo[4,3-c]pyridin-7-yl]-2-oxo-2-[(2R,5S)-5-methyl-2-(6-quinolyl)-1-piperidyl]acetamide (16.2 mg, 28.94 μmol) was dissolved in MeOH (1 mL) and diox/HCl (28.94 μmol, 2 mL) was added. Then mixture was stirred at rt 10 hr. The solution was evaporated and submitted to reverse phase HPLC (column: SunFire C18 100×19 mm, 5 um; mobile phase: 10-40% 2-10 min MeOH, flow rate: 30 ml/min (loading pump 4 ml/min MeOH) to afford N-(4-amino-1H-pyrazolo[4,3-c]pyridin-7-yl)-2-oxo-2-[(2R,5S)-5-methyl-2-(6-quinolyl)-1-piperidyl]acetamide (5.2 mg, 11.16 μmol, 38.56% yield, HCl).

Compound 83: ¹H NMR (600 MHZ, dmso) δ 0.18-1.12 (m, 3H), 1.16-1.47 (m, 1H), 1.54-1.81 (m, 1H), 1.85-2.08 (m, 1H), 2.11-2.35 (m, 1H), 2.60-2.98 (m, 2H), 3.84-4.19 (m, 1H), 5.17-5.93 (m, 1H), 6.27-7.17 (m, 2H), 7.43-7.57 (m, 1H), 7.57-7.71 (m, 1H), 7.71-7.84 (m, 1H), 7.91-8.22 (m, 3H), 8.22-8.42 (m, 1H), 8.66-8.93 (m, 1H), 9.53-10.65 (m, 1H), 12.45-13.51 (m, 1H). LCMS(ESI): [M]⁺ m/z: calcd 429.2; found 430.2; Rt=2.010 min.

### Example 103. Synthesis of N-(4-amino-1H-pyrazolo[4,3-c]pyridin-7-yl)-2-((2R,5S)-5-methyl-2-(naphthalen-2-yl)piperidin-1-yl)-2-oxoacetamide (Compound 79)

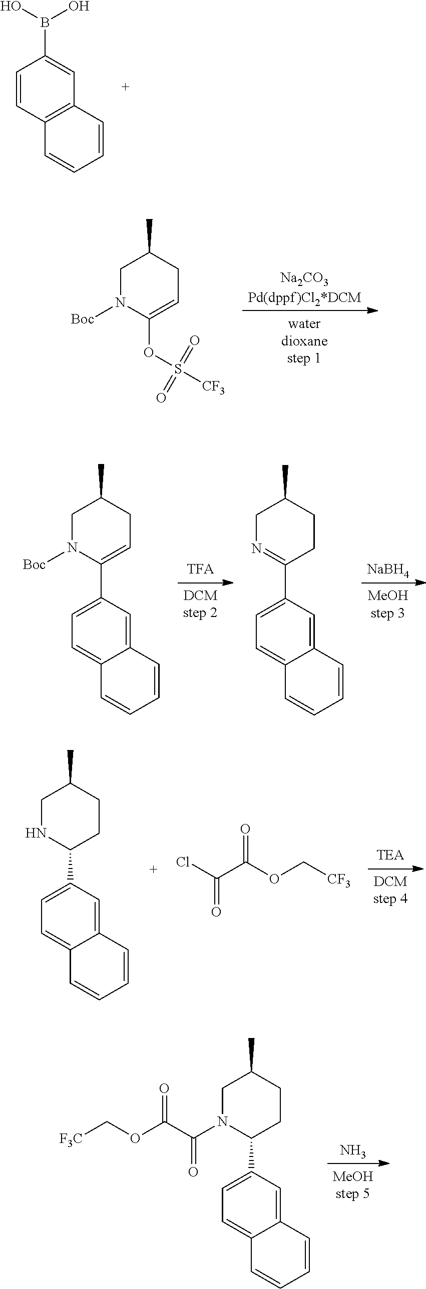

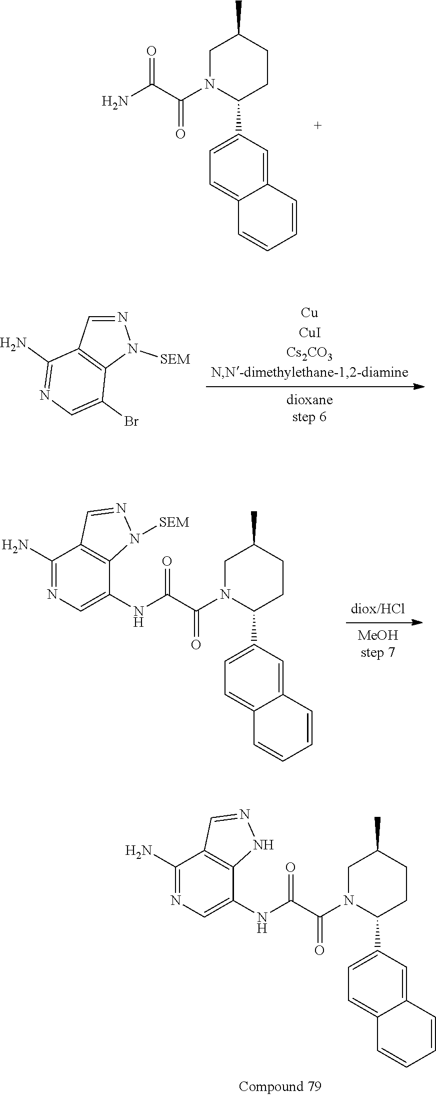 [SMILES: C[C@H]1CC[C@H](c2ccc3ccccc3c2)N(C(=O)C(N)=O)C1 ; C[Si](C)(C)CCOCn1ncc2c(N)ncc(Br)c21 ; C[C@H]1CC[C@H](c2ccc3ccccc3c2)N(C(=O)C(=O)Nc2cnc(N)c3cnn(COCC[Si](C)(C)C)c23)C1 ; C[C@H]1CC[C@H](c2ccc3ccccc3c2)N(C(=O)C(=O)Nc2cnc(N)c3cn[nH]c23)C1]

### Step 1: Synthesis of(S)-tert-butyl 3-methyl-6-(naphthalen-2-yl)-3,4-dihydropyridine-1(2H)-carboxylate

tert-Butyl (3S)-3-methyl-6-(trifluoromethylsulfonyloxy)-3,4-dihydro-2H-pyridine-1-carboxylate (4.82 g, 13.95 mmol), 2-naphthylboronic acid (2.4 g, 13.95 mmol) and sodium carbonate (4.44 g, 41.86 mmol, 1.75 mL) was added to a mixture of dioxane (58.69 mL) and water (19.56 mL). The resulting mixture was evacuated and then backfilled with argon, this operation was repeated three times, then Pd(dppf)Cl₂*DCM (569.79 mg, 697.72 μmol) was added under argon. The reaction mixture was stirred under argon at 70° C. for 16 hr, then cooled and filtered. The filter cake was washed with dioxane (2*20 ml) and discarded. The filtrate was evaporated in vacuum to afford tert-butyl (3S)-3-methyl-6-(2-naphthyl)-3,4-dihydro-2H-pyridine-1-carboxylate (4.1 g, 12.68 mmol, 90.84% yield). LCMS(ESI): [M]⁺ m/z: calcd 323.2; found 324.2; Rt=1.615 min.

### Step 2: Synthesis of(S)-3-methyl-6-(naphthalen-2-yl)-2,3,4,5-tetrahydropyridine

The solution of tert-butyl (3S)-3-methyl-6-(2-naphthyl)-3,4-dihydro-2H-pyridine-1-carboxylate (4.1 g, 12.68 mmol) in TFA (21.68 g, 190.15 mmol, 14.65 mL) was stirred at 25° C. for 2 hr, and then evaporated in vacuum. Crushed ice (15 g) was added to the residue and pH was adjusted to 10 with a 10% aqueous solution of sodium hydroxide. The resulting mixture was extracted with DCM (2*30 ml). The combined organic extracts were dried over sodium sulphate and evaporated in vacuum to afford (3S)-3-methyl-6-(2-naphthyl)-2,3,4,5-tetrahydropyridine (2.2 g, 9.85 mmol, 77.71% yield) as brown oil, which was used directly in the next step. LCMS(ESI): [M]⁺ m/z: calcd 223.2; found 224.2; Rt=0.957 min.

### Step 3: Synthesis of (2R,5S)-5-methyl-2-(naphthalen-2-yl)piperidine

To a stirred solution of (3S)-3-methyl-6-(2-naphthyl)-2,3,4,5-tetrahydropyridine (2.2 g, 9.85 mmol) in MeOH (29.31 mL) was added sodium borohydride (745.43 mg, 19.70 mmol, 694.07 μL) at 0° C. The resulting reaction mixture was stirred at 25° C. for 15 hr. Upon completion, the reaction mixture was concentrated under reduced pressure, and then quenched with water 20 mL and 50 ml EtOAc. The combined organic phase was washed with Brine 20 mL, dried over Na₂SO₄ and concentrated under reduced pressure to obtain (2R,5S)-5-methyl-2-(2-naphthyl)piperidine (1.6 g, 7.10 mmol, 72.08% yield), which was used in next step without farther purification. LCMS(ESI): [M]⁺ m/z: calcd 225.2; found 226.2; Rt=1.003 min.

### Step 4: Synthesis of 2,2,2-trifluoroethyl 2-((2R,5S)-5-methyl-2-(naphthalen-2-yl)piperidin-1-yl)-2-oxoacetate

(2R,5S)-5-Methyl-2-(naphthalen-2-yl)piperidine (200 mg, 887.59 μmol) was dissolved in DCM (9.81 mL) and TEA (134.72 mg, 1.33 mmol, 185.57 μL) was added. Then reaction mixture was cooled and 2,2,2-trifluoroethyl 2-chloro-2-oxo-acetate (202.91 mg, 1.07 mmol) was added dropwise. The reaction was stirred at rt overnight. After that water (50 ml) was added and organic layer was washed with brine (20 ml), dried over Na₂SO₄ and evaporated in vacuum to afford 2,2,2-trifluoroethyl 2-oxo-2-[(2R,5S)-5-methyl-2-(2-naphthyl)-1-piperidyl]acetate (0.3 g, 790.78 μmol, 89.09% yield). LCMS(ESI): [M]⁺ m/z: calcd 379.2; found 380.2; Rt=1.646 min.

### Step 5: Synthesis of 2-((2R,5S)-5-methyl-2-(naphthalen-2-yl)piperidin-1-yl)-2-oxoacetamide

2,2,2-Trifluoroethyl 2-oxo-2-[(2R,5S)-5-methyl-2-(2-naphthyl)-1-piperidyl]acetate (0.3 g, 790.78 μmol) was dissolved in NH₃/MeOH and stirred overnight at rt. Then it was to evaporated in vacuum afford 2-oxo-2-[(2R,5S)-5-methyl-2-(2-naphthyl)-1-piperidyl]acetamide (0.2 g, 674.85 μmol, 85.34% yield). LCMS(ESI): [M]⁺ m/z: calcd 296.2; found 297.2; Rt=1.294 min.

### Step 6: Synthesis of N-(4-amino-1-((2-(trimethylsilyl)ethoxy)methyl)-1H-pyrazolo[4,3-c]pyridin-7-yl)-2-((2R,5S)-5-methyl-2-(naphthalen-2-yl)piperidin-1-yl)-2-oxoacetamide

A mixture of 7-bromo-1-(2-trimethylsilylethoxymethyl)pyrazolo[4,3-c]pyridin-4-amine (115.84 mg, 337.42 μmol), 2-oxo-2-[(2R,5S)-5-methyl-2-(2-naphthyl)-1-piperidyl]acetamide (0.1 g, 337.42 μmol), copper (I) iodide (32.13 mg, 168.71 μmol, 5.72 μL), cesium carbonate (219.88 mg, 674.85 μmol) and rac-(1R,2R)—N1,N2-dimethylcyclohexane-1,2-diamine (24.00 mg, 168.71 μmol) in dioxane (1.99 mL) was stirred in a sealed vial under argon at 95° C. for 16 hr. The resulting mixture was cooled down and submitted to reverse phase HPLC (column: SunFire C18 100×19 mm, 5 um; mobile phase: 10-40% 2-10 min MeOH, flow rate: 30 ml/min (loading pump 4 ml/min MeOH) to afford N-[4-amino-1-(2-trimethylsilylethoxymethyl)pyrazolo[4,3-c]pyridin-7-yl]-2-oxo-2-[(2R,5S)-5-methyl-2-(2-naphthyl)-1-piperidyl]acetamide (13.9 mg, 24.88 μmol, 7.37% yield), which was used directly in the next step. LCMS(ESI): [M]⁺ m/z: calcd 558.2; found 559.2; Rt=3.849 min.

### Step 7: Synthesis of N-(4-amino-1H-pyrazolo[4,3-c]pyridin-7-yl)-2-((2R,5S)-5-methyl-2-(naphthalen-2-yl)piperidin-1-yl)-2-oxoacetamide (Compound 79)

N-[4-Amino-1-(2-trimethylsilylethoxymethyl)pyrazolo[4,3-c]pyridin-7-yl]-2-oxo-2-[(2R,5S)-5-methyl-2-(2-naphthyl)-1-piperidyl]acetamide (13.9 mg, 24.88 μmol) was dissolved in MeOH (1 mL) and diox/HCl (24.88 μmol, 1.5 mL) was added. Then mixture was stirred at rt 10 hr. The solution was evaporated and submitted to reverse phase HPLC (column: SunFire C18 100×19 mm, 5 um; mobile phase: 10-40% 2-10 min MeOH, flow rate: 30 ml/min (loading pump 4 ml/min MeOH) to afford N-(4-amino-1H-pyrazolo[4,3-c]pyridin-7-yl)-2-oxo-2-[(2R,5S)-5-methyl-2-(2-naphthyl)-1-piperidyl]acetamide (3.7 mg, 7.96 μmol, 31.99% yield, HCl).

Compound 79: ¹H NMR (600 MHZ, DMSO-d₆) δ (ppm) 0.78-1.09 (m, 3H), 1.26-1.47 (m, 1H), 1.57-1.80 (m, 1H), 1.84-2.04 (m, 1H), 2.09-2.35 (m, 1H), 2.70-2.87 (m, 1H), 3.79-4.14 (m, 2H), 5.14-6.18 (m, 1H), 6.58-7.21 (m, 2H), 7.33-7.56 (m, 3H), 7.63-7.79 (m, 1H), 7.86-7.95 (m, 3H), 8.08-8.24 (m, 1H), 8.27-8.74 (m, 1H), 9.24-10.62 (m, 1H), 12.62-13.47 (m, 1H). LCMS(ESI): [M]⁺ m/z: calcd 428.2; found 429.2; Rt=2.400 min.

### Example 104. Synthesis of N-(4-amino-1H-pyrazolo[4,3-c]pyridin-7-yl)-2-((2R,5S)-2-(4-fluorophenyl)-5-methylpiperidin-1-yl)-2-oxoacetamide (Compound 101)

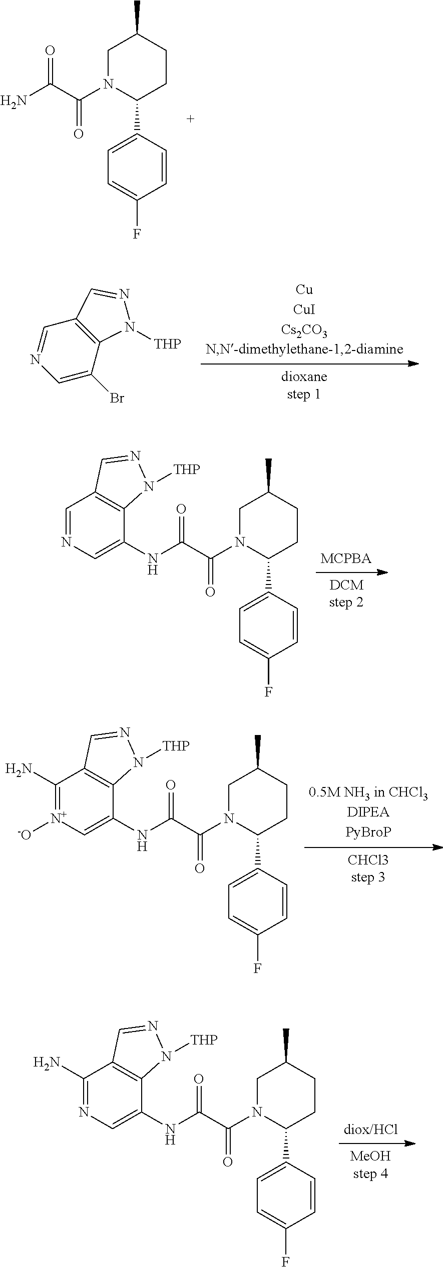

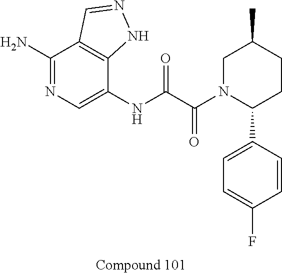 [SMILES: C[C@H]1CC[C@H](c2ccc(F)cc2)N(C(=O)C(=O)Nc2cnc(N)c3cn[nH]c23)C1]

### Step 1: Synthesis of 2-((2R,5S)-2-(4-fluorophenyl)-5-methylpiperidin-1-yl)-2-oxo-N-(1-(tetrahydro-2H-pyran-2-yl)-1H-pyrazolo[4,3-c]pyridin-7-yl)acetamide

2-Oxo-2-[(2R,5S)-2-(4-fluorophenyl)-5-methyl-1-piperidyl]acetamide (160 mg, 605.38 μmol), 7-bromo-1-tetrahydropyran-2-yl-pyrazolo[4,3-c]pyridine (170.80 mg, 605.38 μmol), copper (I) iodide (115.30 mg, 605.38 μmol, 20.52 μL), cesium carbonate (394.49 mg, 1.21 mmol) and (1R,2R)—N1,N2-dimethylcyclohexane-1,2-diamine (129.16 mg, 908.08 μmol) were mixed in dioxane (5 mL) under argon, and then stirred overnight at 100° C. for 14 hr in vial. The reaction mixture was filtered and the filtrate was concentrated in vacuum and the residue was purified by HPLC (SYSTEM 2-10 min 40-50% MeOH+NH₃ 30 ml/min (loading pump 4 ml MeOH) column: SunFire 100*19 mm, 5 microM) to give 2-oxo-2-[(2R,5S)-2-(4-fluorophenyl)-5-methyl-1-piperidyl]-N-(1-tetrahydropyran-2-ylpyrazolo[4,3-c]pyridin-7-yl)acetamide (32 mg, 68.74 μmol, 11.35% yield). LCMS(ESI): [M]⁺ m/z: calcd 465.2; found 466.2; Rt=1.312 min.

### Step 2: Synthesis of 4-amino-7-(2-((2R,5S)-2-(4-fluorophenyl)-5-methylpiperidin-1-yl)-2-oxoacetamido)-1-(tetrahydro-2H-pyran-2-yl)-1H-pyrazolo[4,3-c]pyridine 5-oxide

To a stirred solution of 2-oxo-2-[(2R,5S)-2-(4-fluorophenyl)-5-methyl-1-piperidyl]-N-(1-tetrahydropyran-2-ylpyrazolo[4,3-c]pyridin-7-yl)acetamide (32 mg, 68.74 μmol) in DCM (5 mL) was added portion wise MCPBA (14.24 mg, 82.49 μmol) and the resulting mixture was stirred at 20° C. for 16 hr. After 16 hr the reaction mixture was concentrated under reduced pressure. The obtained residue was diluted with water and the resulting precipitate was filtered. The filtrate was concentrated and dried under reduced pressure to obtain N-(5-oxido-1-tetrahydropyran-2-yl-pyrazolo[4,3-c]pyridin-5-ium-7-yl)-2-oxo-2-[(2R,5S)-2-(4-fluorophenyl)-5-methyl-1-piperidyl]acetamide (30 mg, crude) as a yellow solid. LCMS(ESI): [M]⁺ m/z: calcd 481.2; found 482.2; Rt=1.331 min.

### Step 3: Synthesis of N-(4-amino-1-(tetrahydro-2H-pyran-2-yl)-1H-pyrazolo[4,3-c]pyridin-7-yl)-2-((2R,5S)-2-(4-fluorophenyl)-5-methylpiperidin-1-yl)-2-oxoacetamide

Combine N-(5-oxido-1-tetrahydropyran-2-yl-pyrazolo[4,3-c]pyridin-5-ium-7-yl)-2-oxo-2-[(2R,5S)-2-(4-fluorophenyl)-5-methyl-1-piperidyl]acetamide (30 mg, 62.30 μmol), 0.5M NH₃ in CHCl₃ (62.30 μmol, 1 mL), DIPEA (30.20 mg, 233.64 μmol, 40.70 μL) and PyBroP (37.76 mg, 80.99 μmol) and stir for 16 hr at rt. The solvent was evaporated to obtain N-(4-amino-1-tetrahydropyran-2-yl-pyrazolo[4,3-c]pyridin-7-yl)-2-oxo-2-[(2R,5S)-2-(4-fluorophenyl)-5-methyl-1-piperidyl]acetamide (67 mg, crude). LCMS(ESI): [M]⁺ m/z: calcd 480.2; found 481.2; Rt=1.067 min.

### Step 4: Synthesis of N-(4-amino-1H-pyrazolo[4,3-c]pyridin-7-yl)-2-((2R,5S)-2-(4-fluorophenyl)-5-methylpiperidin-1-yl)-2-oxoacetamide (Compound 101)

To a solution of N-(4-amino-1-tetrahydropyran-2-yl-pyrazolo[4,3-c]pyridin-7-yl)-2-oxo-2-[(2R,5S)-2-(4-fluorophenyl)-5-methyl-1-piperidyl]acetamide (67.00 mg, 139.43 μmol) in MeOH (2 mL) was added hydrogen chloride solution 4.0M in dioxane (800.00 mg, 21.94 mmol, 1 mL) at 25° C. The resulting mixture was left to stir for 15 hr. The resulting mixture was evaporated to dryness and then submitted to reverse phase HPLC (2-10 min 25-50% water MeOH+NH₃ 30/min; loading pump 4 ml/min MeOH; column SunFire 19*100 mm) to afford N-(4-amino-1H-pyrazolo[4,3-c]pyridin-7-yl)-2-oxo-2-[(2R,5S)-2-(4-fluorophenyl)-5-methyl-1-piperidyl]acetamide (27 mg, 68.11 μmol, 48.85% yield)

Compound 101: ¹H NMR (600 MHZ, DMSO-d₆) δ (ppm) 0.99 (m, 3H), 1.32 (m, 1H), 1.70 (m, 1H), 1.89 (m, 1H), 2.05 (m, 1H), 2.18 (m, 1H), 2.72 (m, 1H), 3.89 (m, 1H), 5.43 (m, 1H), 6.74 (m, 2H), 7.25 (m, 4H), 7.60 (m, 1H), 8.17 (m, 1H), 9.92 (m, 1H), 13.04 (m, 1H).

LCMS(ESI): [M]⁺ m/z: calcd 396.2; found 397.2; Rt=1.000 min.

### Example 105. Synthesis of N-(4-amino-1H-pyrazolo[4,3-c]pyridin-7-yl)-2-[(2R,5S)-2-(1,3-benzothiazol-5-yl)-5-methyl-1-piperidyl]-2-oxo-acetamide (Compound 17)

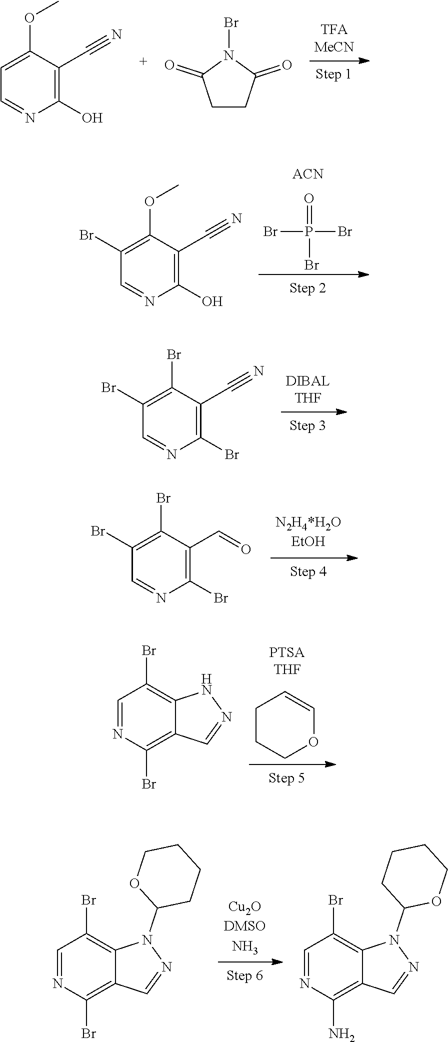

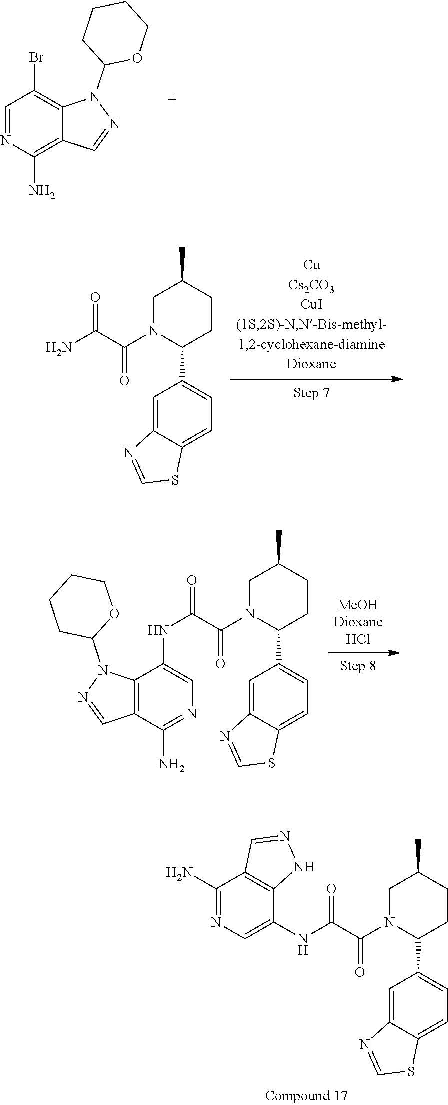

### Step 1: The synthesis of 5-bromo-2-hydroxy-4-methoxy-pyridine-3-carbonitrile

A mixture of 2-hydroxy-4-methoxy-pyridine-3-carbonitrile (5.00 g, 33.3 mmol), NBS (7.11 g, 39.9 mmol, 3.39 mL) and TFA (949 mg, 8.33 mmol, 641 μL) in ACN (125 mL) was refluxed for 8 hr. The reaction mixture was filtered, washed with ACN (25.0 mL). The filter cake was dried to afford 5-bromo-2-hydroxy-4-methoxy-pyridine-3-carbonitrile (6.14 g, 26.8 mmol, 80.5% yield) as a white solid. LCMS(ESI): [M+H]⁺ m/z: calcd 228.95 and 230.95; found 230.0; Rt=1.623.

### Step 2 The synthesis of 2,4,5-tribromopyridine-3-carbonitrile

To a solution of 5-bromo-2-hydroxy-4-methoxy-pyridine-3-carbonitrile (7.34 g, 32.1 mmol) in ACN (250 mL) Phosphorus (V) oxybromide (18.4 g, 64.1 mmol, 6.52 mL) was added. The reaction mixture was heated to 80° C. for 24 hr. The obtained mixture was cooled to RT and poured into saturated K₂CO₃ solution. The resulting moisture was extracted with EtOAc (2×250 mL), washed with brine, dried over Na₂SO₄ and concentrated in vacuo to afford 2,4,5-tribromopyridine-3-carbonitrile (9.40 g, 27.6 mmol, 86.1% yield) as a beige solid.

LCMS(ESI): [M+H]⁺ m/z: calcd 340.77 and 342.77; found 342.8; Rt=3.543.

### Step 3: The synthesis of 2,4,5-tribromopyridine-3-carbaldehyde

To a stirred solution of 2,4,5-tribromopyridine-3-carbonitrile (9.40 g, 27.6 mmol) in toluene (500 mL) DIBAL (5.10 g, 35.9 mmol, 35.9 mL) was added dropwise at −40° C. The reaction mixture was stirred for 1 hour. The obtained mixture was quenched with a cold 1N HCl solution (20.0 mL). The resulting mixture was stirred for 1 hr. EtOAc (20.0 mL) was added. The organic layer was separated. The aqueous layer was extracted with EtOAc (10 mL×2). The combined organic layers were dried over anhydrous Na₂SO₄ and concentrated under reduced pressure to afford 2,4,5-tribromopyridine-3-carbaldehyde (6.60 g, 19.2 mmol, 69.6% yield) as a beige solid. LCMS(ESI): [M+H]⁺ m/z: calcd 277.87; found 277.8; Rt=2.342.

### Step 4: The synthesis of 4,7-dibromo-1H-pyrazolo[4,3-c]pyridine

To a previously heated (to 70° C.) solution of 2,4,5-tribromopyridine-3-carbaldehyde (5.60 g, 16.3 mmol) in EtOH (48.7 mL) Hydrazine monohydrate (1.63 g, 32.6 mmol, 1.59 mL) was added. The resulting mixture was refluxed for 45 min. The reaction solution was concentrated in vacuo to dryness. The residue was diluted with EtOAc and water. The organic layers were dried over Na₂SO₄, filtered and concentrated in vacuo to afford 4,7-dibromo-1H-pyrazolo[4,3-c]pyridine (4.20 g, 15.2 mmol, 93.1% yield) as an orange solid. LCMS(ESI): [M+H]⁺ m/z: calcd 277.87; found 277.8; Rt=2.342.

### Step 5: The synthesis of 4,7-dibromo-1-tetrahydropyran-2-yl-pyrazolo[4,3-c]pyridine

To a solution of 4,7-dibromo-1H-pyrazolo[4,3-c]pyridine (4.20 g, 15.2 mmol), 3,4-dihydro-2H-pyran (3.83 g, 45.5 mmol, 4.13 mL) and p-Toluenesulfonic acid monohydrate (289 mg, 1.52 mmol, 233 μL) in THF (122 mL) was stirred at 65° C. for 48 hr. The reaction mixture was concentrated in vacuo. The residue was purified by CC(SiO₂, petroleum ether/ethyl acetate with ethyl acetate from 20˜30%, flow rate=100 mL/min, Rv=5.7-6.5 CV) to afford 4,7-dibromo-1-tetrahydropyran-2-yl-pyrazolo[4,3-c]pyridine (2.90 g, 8.03 mmol, 52.9% yield) as an off-white solid. LCMS(ESI): [M-THP]⁺ m/z: calcd 275.86; found 275.8; Rt=1.314.

### Step 6: The synthesis of 7-bromo-1-tetrahydropyran-2-yl-pyrazolo[4,3-c]pyridin-4-amine

A solution of 4,7-dibromo-1-tetrahydropyran-2-yl-pyrazolo[4,3-c]pyridine (1.00 g, 2.77 mmol) and Copper (I) oxide (39.6 mg, 277 μmol, 6.61 μL) in DMSO (20.0 mL) was purged with NH₃ (47.17 mg, 2.77 mmol)(gas) in a sealed tube for 10 min. The reaction mixture was stirred at 100° C. overnight. The obtained mixture was filtered. The filtrate was subjected to HPLC (0-1-6 min 10-10-30% water-ACN, +0.1% vol. of 25% aq. NH₃, 30 mL/min, column: XBridge BEH C18, 100×20 mm, 5 μm) to afford 7-bromo-1-tetrahydropyran-2-yl-pyrazolo[4,3-c]pyridin-4-amine (333 mg, 1.12 mmol, 40.4% yield) as a green solid.

LCMS(ESI): [M+H]⁺ m/z: calcd 297.04 and 299.03; found 299.0; Rt=1.972.

### Step 7: The synthesis of N-(4-amino-1-tetrahydropyran-2-yl-pyrazolo[4,3-c]pyridin-7-yl)-2-[(2R,5S)-2-(1,3-benzothiazol-5-yl)-5-methyl-1-piperidyl]-2-oxo-acetamide

7-bromo-1-tetrahydropyran-2-yl-pyrazolo[4,3-c]pyridin-4-amine (0.15 g, 512 μmol), 2-[(2R,5S)-2-(1,3-benzothiazol-5-yl)-5-methyl-1-piperidyl]-2-oxo-acetamide (155 mg, 512 μmol), Cu (32.5 mg, 512 μmol), Cesium carbonate (333 mg, 1.02 mmol), copper (I) iodide (97.4 mg, 512 μmol, 17.3 μL) and (1S,2S)—N,N′-Bis-methyl-1,2-cyclohexane-diamine (109 mg, 767 μmol, 121 μL) were mixed in Dioxane in an inert atmosphere. The resulting mixture was allowed to stir at 100° C. for 15 hr in a vial. The reaction mixture was concentrated in vacuo. The residue was subjected to HPLC (0-1-6 min 40-40-65% water-methanol, +0.1% vol. of 25% aq. NH₃, 30 mL/min, column: YMC-Actus Triart C18, 100×20 mm, 5 μm) to afford N-(4-amino-1-tetrahydropyran-2-yl-pyrazolo[4,3-c]pyridin-7-yl)-2-[(2R,5S)-2-(1,3-benzothiazol-5-yl)-5-methyl-1-piperidyl]-2-oxo-acetamide (0.04 g, crude) as a brown solid.

LCMS(ESI): [M+H]⁺ m/z: calcd 520.23; found 520.2; Rt=2.667.

### Step 8: The synthesis of N-(4-amino-1H-pyrazolo[4,3-c]pyridin-7-yl)-2-[(2R,5S)-2-(1,3-benzothiazol-5-yl)-5-methyl-1-piperidyl]-2-oxo-acetamide

N-(4-amino-1-tetrahydropyran-2-yl-pyrazolo[4,3-c]pyridin-7-yl)-2-[(2R,5S)-2-(1,3-benzothiazol-5-yl)-5-methyl-1-piperidyl]-2-oxo-acetamide (0.05 g, 99.1 μmol) was dissolved in a mixture of MeOH (2.00 mL) and Dioxane/HCl (2.00 mL). The resulting solution was stirred for 16 hr at 25° C. The reaction mixture was concentrated in vacuo. The crude residue was subjected to HPLC (0-1-6 min 10-10-50% water+FA (0.1% vol.)—ACN+FA (0.1% vol.); flow: 30 mL/min, column: Chromatorex 18 SMB100-5T, 100×19 mm, 5 μm) to afford N-(4-amino-1H-pyrazolo[4,3-c]pyridin-7-yl)-2-[(2R,5S)-2-(1,3-benzothiazol-5-yl)-5-methyl-1-piperidyl]-2-oxo-acetamide (0.004 g, 10.33 μmol, 10.43% yield) as a yellow solid. ¹H NMR (600 MHz, DMSO) δ 0.80-1.06 (m, 3H), 1.29-1.48 (m, 1H), 1.71-1.81 (m, 1H), 1.86-2.01 (m, 1H), 2.09-2.35 (m, 2H), 2.79-2.97 (m, 1H), 3.79-4.14 (m, 1H), 5.16-5.83 (m, 1H), 6.52-6.86 (m, 2H), 7.44-7.57 (m, 1H), 7.59-7.78 (m, 1H), 8.04-8.14 (m, 1H), 8.17-8.20 (m, 1H), 8.64-8.83 (m, 1H), 9.22-9.37 (m, 1H), 9.37-9.44 (m, 1H), 10.50-10.75 (m, 1H), 12.66-13.42 (m, 1H). LCMS(ESI): [M+H]⁺ m/z: calcd 436.16; found 436.2; Rt=2.176.

### Example 106. Synthesis of N-(4-amino-1H-pyrazolo[4,3-c]pyridin-7-yl)-2-((2R,5S)-5-methyl-2-(2-(tetrahydro-2H-pyran-4-yl)benzo[d]thiazol-5-yl)piperidin-1-yl)-2-oxoacetamide (Compound 143)

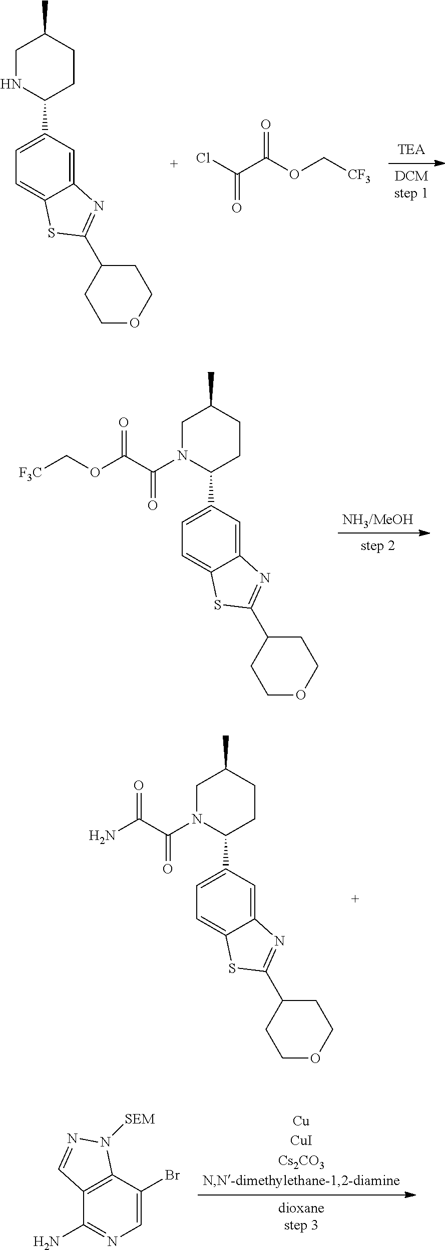 [SMILES: C[C@H]1CC[C@H](c2ccc3sc(C4CCOCC4)nc3c2)NC1 ; O=C(Cl)C(=O)OCC(F)(F)F ; C[C@H]1CC[C@H](c2ccc3sc(C4CCOCC4)nc3c2)N(C(=O)C(=O)OCC(F)(F)F)C1 ; "NH3/MeOH" ; C[C@H]1CC[C@H](c2ccc3sc(C4CCOCC4)nc3c2)N(C(=O)C(N)=O)C1 ; C[Si](C)(C)CCOCn1ncc2c(N)ncc(Br)c21]

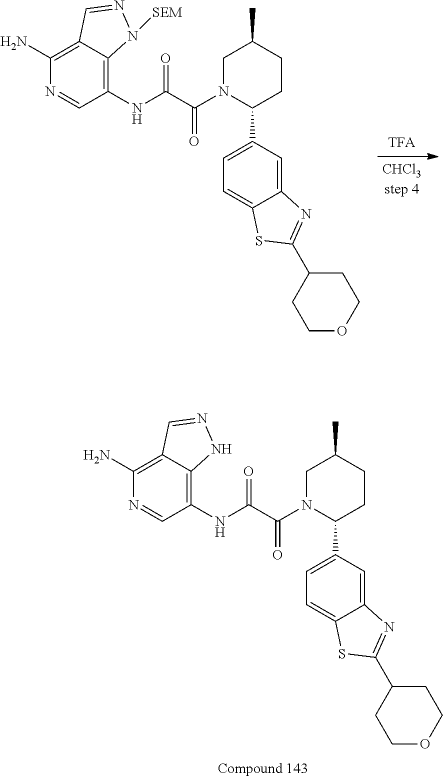 [SMILES: C[C@H]1CC[C@H](c2ccc3sc(C4CCOCC4)nc3c2)N(C(=O)C(=O)Nc2cnc(N)c3cnn(COCC[Si](C)(C)C)c23)C1 ; C[C@H]1CC[C@H](c2ccc3sc(C4CCOCC4)nc3c2)N(C(=O)C(=O)Nc2cnc(N)c3cn[nH]c23)C1]

The synthesis of 5-((2R,5S)-5-methylpiperidin-2-yl)-2-(tetrahydro-2H-pyran-4-yl)benzo[d]thiazole is give by General Scheme S2, step 5.

### Step 1: Synthesis of 2,2,2-trifluoroethyl 2-((2R,5S)-5-methyl-2-(2-(tetrahydro-2H-pyran-4-yl)benzo[d]thiazol-5-yl)piperidin-1-yl)-2-oxoacetate

The synthesis of 2,2,2-trifluoroethyl 2-((2R,5S)-5-methyl-2-(2-(tetrahydro-2H-pyran-4-yl)benzo[d]thiazol-5-yl)piperidin-1-yl)-2-oxoacetate is given by General Scheme S3 step 1.

Yield: 0.2 g of crude. LCMS(ESI): [M]⁺ m/z: calcd 470.2; found 471.2; Rt=1.377 min.

### Step 2: Synthesis of 2-((2R,5S)-5-methyl-2-(2-(tetrahydro-2H-pyran-4-yl)benzo[d]thiazol-5-yl)piperidin-1-yl)-2-oxoacetamide

The synthesis of 2-((2R,5S)-5-methyl-2-(2-(tetrahydro-2H-pyran-4-yl)benzo[d]thiazol-5-yl)piperidin-1-yl)-2-oxoacetamide is given by General Scheme S3, Step 2. Yield: 0.15 g of crude. LCMS(ESI): [M]⁺ m/z: calcd 387.2; found 388.2; Rt=1.208 min.

### Step 3: Synthesis of N-(4-amino-1-((2-(trimethylsilyl)ethoxy)methyl)-1H-pyrazolo[4,3-c]pyridin-7-yl)-2-((2R,5S)-5-methyl-2-(2-(tetrahydro-2H-pyran-4-yl)benzo[d]thiazol-5-yl)piperidin-1-yl)-2-oxoacetamide

The synthesis of N-(4-amino-1-((2-(trimethylsilyl)ethoxy)methyl)-1H-pyrazolo[4,3-c]pyridin-7-yl)-2-((2R,5S)-5-methyl-2-(2-(tetrahydro-2H-pyran-4-yl)benzo[d]thiazol-5-yl)piperidin-1-yl)-2-oxoacetamide is given by General Scheme S3, Step 3. Yield: 80 mg of crude. LCMS(ESI): [M]⁺ m/z: calcd 649.2; found 650.2; Rt=1.263 min.

### Step 4: Synthesis of N-(4-amino-1H-pyrazolo[4,3-c]pyridin-7-yl)-2-((2R,5S)-5-methyl-2-(2-(tetrahydro-2H-pyran-4-yl)benzo[d]thiazol-5-yl)piperidin-1-yl)-2-oxoacetamide (Compound 143)

N-[4-Amino-1-(2-trimethylsilylethoxymethyl)pyrazolo[4,3-c]pyridin-7-yl]-2-oxo-2-[(2R,5S)-5-methyl-2-(2-tetrahydropyran-4-yl-1,3-benzothiazol-5-yl)-1-piperidyl]acetamide (0.08 g, 123.10 μmol) and TFA (148.00 mg, 1.30 mmol, 0.1 mL) were stirred in CHCl₃ (5 mL) for 16 hr at 25° C. Upon completion, the reaction mixture was concentrated under reduced pressure. The obtained crude product was purified by reverse phase HPLC chromatography (Device (Mobile Phase, Column): SYSTEM 30-80% 0-5 min H₂O/MeOH/0.1% NH₄OH, flow: 30 ml/min (loading pump 4 ml/min MeOH) target mass 519.63 column: XBridge C18 100×19 mm, 5 um) to afford product N-(4-amino-1H-pyrazolo[4,3-c]pyridin-7-yl)-2-oxo-2-[(2R,5S)-5-methyl-2-(2-tetrahydropyran-4-yl-1,3-benzothiazol-5-yl)-1-piperidyl]acetamide (8 mg, 15.40 μmol, 12.51% yield).

Compound 143: ¹H NMR (600 MHz, DMSO-d₆) δ (ppm) 0.91-1.07 (m, 3H), 1.26-1.47 (m, 1H), 1.72-1.93 (m, 4H), 2.00-2.05 (m, 2H), 2.11-2.34 (m, 1H), 2.78-3.06 (m, 1H), 3.37-3.43 (m, 2H), 3.47-3.52 (m, 2H), 3.74-3.84 (m, 0.7H), 3.91-3.95 (m, 2H), 4.07-4.13 (m, 0.3H), 5.13-5.89 (m, 1H), 6.61-7.02 (m, 2H), 7.28-7.48 (m, 1H), 7.54-7.73 (m, 1H), 7.78-7.95 (m, 1H), 7.97-8.10 (m, 1H), 8.13-8.25 (m, 1H), 9.56-10.63 (m, 1H), 12.66-13.42 (m, 1H). LCMS(ESI): [M]⁺ m/z: calcd 519.2; found 520.2; Rt=2.415 min.

### Example 107. Synthesis of N-(4-amino-1H-pyrazolo[4,3-c]pyridin-7-yl)-2-((2R,5S)-5-methyl-2-(2-methylbenzo[d]thiazol-5-yl)piperidin-1-yl)-2-oxoacetamide (Compound 181)

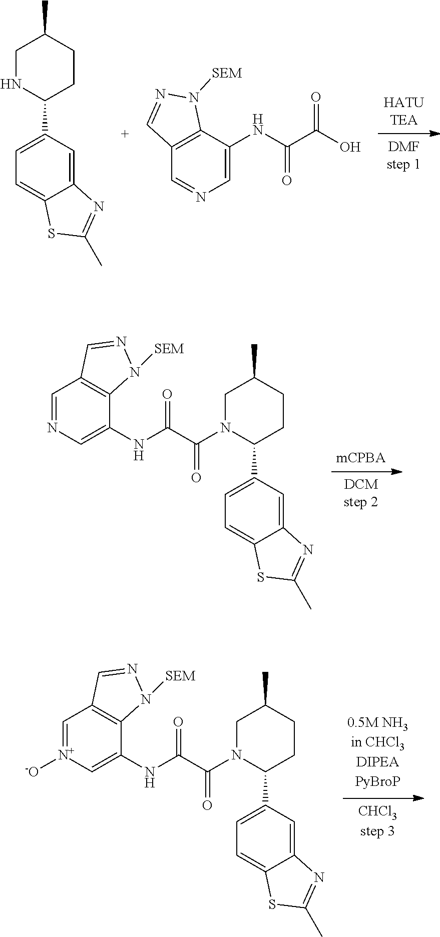

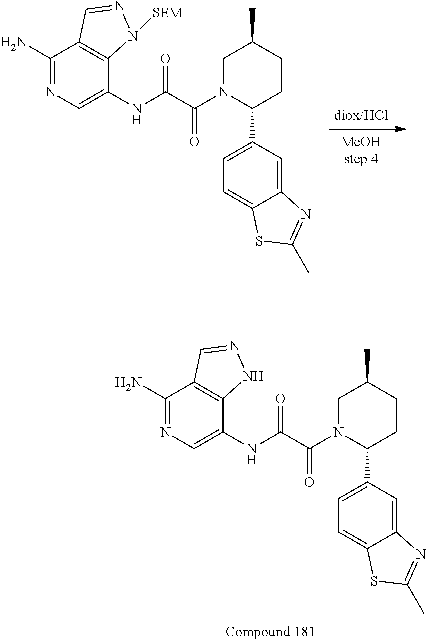 [SMILES: Cc1nc2cc([C@H]3CC[C@H](C)CN3C(=O)C(=O)Nc3cnc(N)c4cnn(COCC[Si](C)(C)C)c34)ccc2s1 ; Cc1nc2cc([C@H]3CC[C@H](C)CN3C(=O)C(=O)Nc3cnc(N)c4cn[nH]c34)ccc2s1]

The synthesis of 2-methyl-5-((2R,5S)-5-methylpiperidin-2-yl)benzo[d]thiazole is given by General Scheme S2, Step 5.

### Step 1: Synthesis of 2-((2R,5S)-5-methyl-2-(2-methylbenzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

The synthesis of 2-((2R,5S)-5-methyl-2-(2-methylbenzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide is given by General Scheme S2, step 6A. Yield: 430.8 mg (31.32%). HPLC conditions: Column: SunFire C18 100*19 mm, 5 microM; 2-10 min 30-80% MeOH+FA, flow: 30 ml/min; (loading pump 4 ml/min MeOH). LCMS(ESI): [M]⁺ m/z: calcd 564.2; found 565.2; Rt=1.414 min.

### Step 2: Synthesis of 7-(2-((2R,5S)-5-methyl-2-(2-methylbenzo[d]thiazol-5-yl)piperidin-1-yl)-2-oxoacetamido)-1-((2-(trimethylsilyl)ethoxy)methyl)-1H-pyrazolo[4,3-c]pyridine 5-oxide

To a stirred solution of 2-oxo-2-[(2R,5S)-5-methyl-2-(2-methyl-1,3-benzothiazol-5-yl)-1-piperidyl]-N-[1-(2-trimethylsilylethoxymethyl)pyrazolo[4,3-c]pyridin-7-yl]acetamide (291.5 mg, 516.14 μmol) in DCM (10 mL) was added portion wise m-CPBA (133.60 mg, 774.20 μmol) and the resulting mixture was stirred at 20° C. for 12 hr. After 12 hr, the reaction mixture was washed with 10% aqueous NaHSO₃ solution and saturated aqueous NaHCO₃ solution. The organic layer was separated, dried over sodium sulfate and concentrated in vacuum to afford N-[5-oxido-1-(2-trimethylsilylethoxymethyl)pyrazolo[4,3-c]pyridin-5-ium-7-yl]-2-oxo-2-[(2R,5S)-5-methyl-2-(2-methyl-1,3-benzothiazol-5-yl)-1-piperidyl]acetamide (266 mg, crude). LCMS(ESI): [M]⁺ m/z: calcd 580.2; found 581.2; Rt=1.432 min.

### Step 3: Synthesis of N-(4-amino-1-((2-(trimethylsilyl)ethoxy)methyl)-1H-pyrazolo[4,3-c]pyridin-7-yl)-2-((2R,5S)-5-methyl-2-(2-methylbenzo[d]thiazol-5-yl)piperidin-1-yl)-2-oxoacetamide

Combine N-[5-oxido-1-(2-trimethylsilylethoxymethyl)pyrazolo[4,3-c]pyridin-5-ium-7-yl]-2-oxo-2-[(2R,5S)-5-methyl-2-(2-methyl-1,3-benzothiazol-5-yl)-1-piperidyl]acetamide (266 mg, 458.01 μmol), 0.5M NH₃ in CHCl₃ (458.01 μmol), DIPEA (221.97 mg, 1.72 mmol, 299.16 μL) and PyBroP (277.57 mg, 595.41 μmol) and stir for 12 hr at rt. The solvent was evaporated to obtain. The resulting crude product was purified by HPLC (2-10 min 10-50% MeCN+FA, 30 ml/min) to obtain N-[4-amino-1-(2-trimethylsilylethoxymethyl)pyrazolo[4,3-c]pyridin-7-yl]-2-oxo-2-[(2R,5S)-5-methyl-2-(2-methyl-1,3-benzothiazol-5-yl)-1-piperidyl]acetamide (12.8 mg, crude). LCMS(ESI): [M]⁺ m/z: calcd 579.2; found 580.2; Rt=1.198 min.

### Step 4: Synthesis of N-(4-amino-1H-pyrazolo[4,3-c]pyridin-7-yl)-2-((2R,5S)-5-methyl-2-(2-methylbenzo[d]thiazol-5-yl)piperidin-1-yl)-2-oxoacetamide (Compound 181)

A solution of N-[4-amino-1-(2-trimethylsilylethoxymethyl)pyrazolo[4,3-c]pyridin-7-yl]-2-oxo-2-[(2R,5S)-5-methyl-2-(2-methyl-1,3-benzothiazol-5-yl)-1-piperidyl]acetamide (12.8 mg, 22.08 μmol) in MeOH (1 mL) and hydrogen chloride solution 4.0M in dioxane (1 mL) was stirred at rt for 12 hr. Solvents were evaporated. The resulting crude product was purified by HPLC (5-40% 0-1-5 min H₂O/MeCN/0.2% FA, flow: 30 ml/min) to obtain N-(4-amino-1H-pyrazolo[4,3-c]pyridin-7-yl)-2-oxo-2-[(2R,5S)-5-methyl-2-(2-methyl-1,3-benzothiazol-5-yl)-1-piperidyl]acetamide (6.1 mg, 13.57 μmol, 61.47% yield). LCMS(ESI): [M]⁺ m/z: calcd 449.2; found 450.2; Rt=0.885 min.

### Example 108. Synthesis of 2-((2R,5S)-2-(2-((1,4-dimethylpiperidin-4-yl)methyl)benzo[d]thiazol-5-yl)-5-methylpiperidin-1-yl)-2-oxo-N-(1H-pyrazolo[4,3-c]pyridin-7-yl)acetamide (Compound 142)

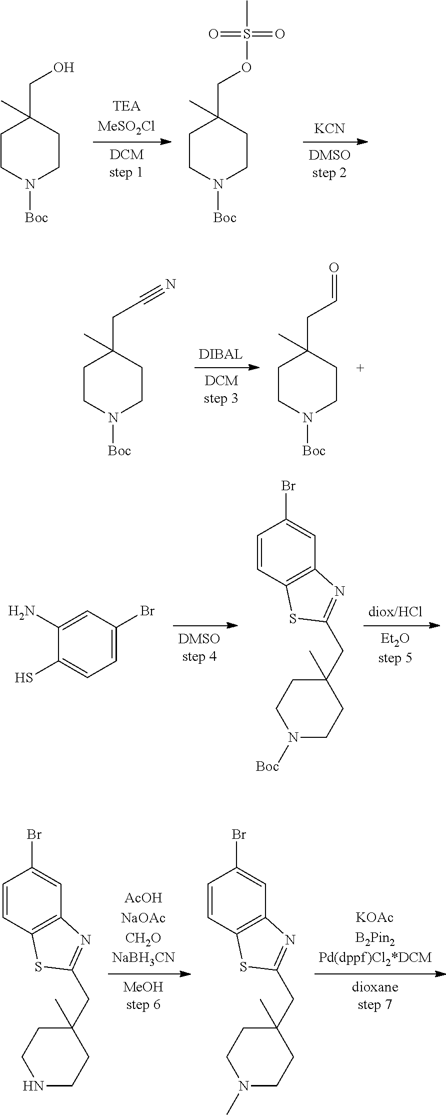

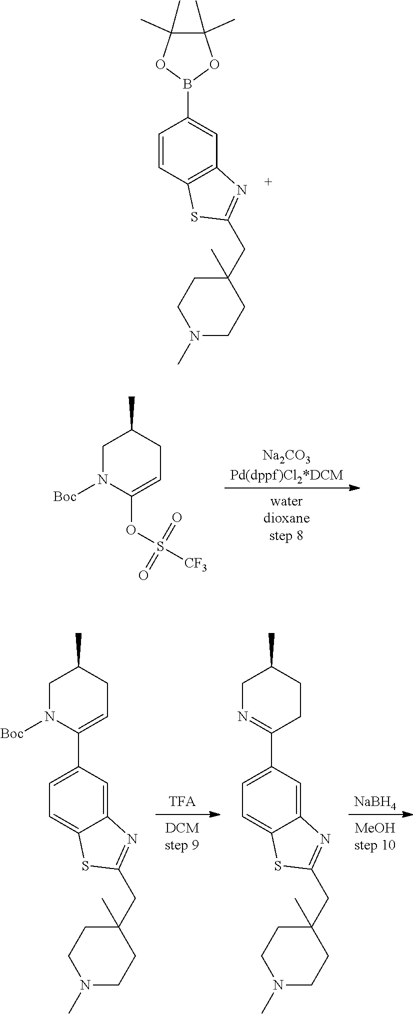

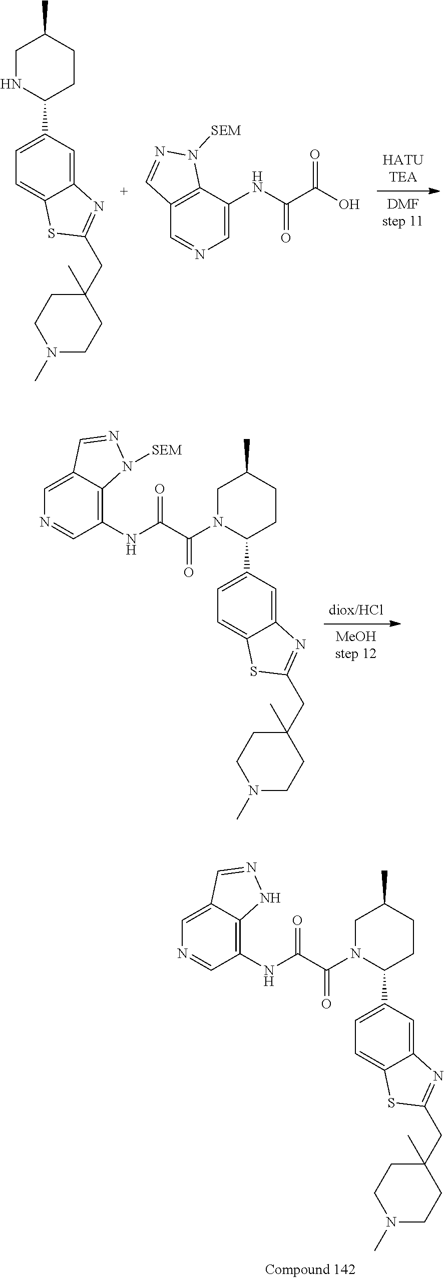 [SMILES: C[C@H]1CC[C@H](c2ccc3sc(CC4(C)CCN(C)CC4)nc3c2)NC1 ; C[Si](C)(C)CCOCn1ncc2cncc(NC(=O)C(=O)O)c21 ; C[C@H]1CC[C@H](c2ccc3sc(CC4(C)CCN(C)CC4)nc3c2)N(C(=O)C(=O)Nc2cncc3cnn(COCC[Si](C)(C)C)c23)C1 ; C[C@H]1CC[C@H](c2ccc3sc(CC4(C)CCN(C)CC4)nc3c2)N(C(=O)C(=O)Nc2cncc3cn[nH]c23)C1]

### Step 1: Synthesis of tert-butyl 4-methyl-4-(((methylsulfonyl)oxy)methyl)piperidine-1-carboxylate

To a solution of tert-butyl 4-(hydroxymethyl)-4-methyl-piperidine-1-carboxylate (8.97 g, 35.20 mmol), TEA (4.27 g, 42.25 mmol, 5.89 mL) in DCM (17.21 mL) was added methansulfonyl chloride (4.44 g, 38.73 mmol, 3.00 mL) portion wise at 0° C. and allowed to warm to room temperature. The resulting solution was washed with 10% aq. HCl and brine, dried over Na₂SO₄ and evaporated to dryness to give tert-butyl 4-methyl-4-(methylsulfonyloxymethyl)piperidine-1-carboxylate (9 g, 29.28 mmol, 83.16% yield) as a brownish gum.

¹H NMR (500 MHZ, CDCl₃) δ (ppm) 1.05 (s, 3H), 1.34 (m, 2H), 1.43 (s, 9H), 1.48 (m, 2H), 2.99 (s, 3H), 3.14 (m, 2H), 3.66 (m, 2H), 3.94 (s, 2H).

### Step 2: Synthesis of tert-butyl 4-(cyanomethyl)-4-methylpiperidine-1-carboxylate

To a solution of tert-butyl 4-methyl-4-(methylsulfonyloxymethyl)piperidine-1-carboxylate (10 g, 32.53 mmol) in DMSO (75 mL) was added potassium cyanide (8.47 g, 130.12 mmol) and the resulting mixture was heated to 130° C. for 12 hr. The resulting mixture was poured into water, extracted with EtOAc (3×100 mL), combined organics were washed with water, brine, dried and evaporated to give tert-butyl 4-(cyanomethyl)-4-methyl-piperidine-1-carboxylate (7.3 g, crude) as a yellow gum.

¹H NMR (500 MHz, CDCl₃) δ (ppm) 1.14 (s, 3H), 1.44 (s, 9H), 1.46 (m, 4H), 2.29 (s, 2H), 3.22 (m, 2H), 3.57 (m, 2H).

### Step 3: Synthesis of tert-butyl 4-methyl-4-(2-oxoethyl)piperidine-1-carboxylate

To a solution of tert-butyl 4-(cyanomethyl)-4-methyl-piperidine-1-carboxylate (6.93 g, 29.08 mmol) in DCM (250 mL) at −30° C. was added DIBAL (10.34 g, 72.69 mmol, 72.69 mL) slowly, and the mixture was stirred at the same temperature for 30 min. 15 mL of Methanol was added followed by 25 mL of the saturated citric acid solution, and the reaction mixture was stirred allowed at rt for 15 min. The reaction mixture was filtered through a pad of celite, and the filtrate was diluted with 250 mL of DCM. The organic layer was washed with brine solution and concentrated in vacuum to give tert-butyl 4-methyl-4-(2-oxoethyl)piperidine-1-carboxylate (4.2 g, crude) as a light yellow oil.

¹H NMR (500 MHz, DMSO-d₆) δ (ppm) 1.13 (s, 3H), 1.40 (s, 9H), 1.52 (m, 4H), 2.34 (s, 2H), 3.29 (m, 2H), 3.51 (m, 2H), 9.84 (s, 1H).

### Step 4: Synthesis of tert-butyl 4-((5-bromobenzo[d]thiazol-2-yl)methyl)-4-methylpiperidine-1-carboxylate

The synthesis of tert-butyl 4-((5-bromobenzo[d]thiazol-2-yl)methyl)-4-methylpiperidine-1-carboxylate is given by General Scheme S2, Step 1B. Yield: 4.8 g (81.05%).

¹H NMR (500 MHz, CDCl₃) δ (ppm) 1.08 (s, 3H), 1.43 (s, 9H), 1.46 (m, 2H), 1.57 (m, 2H), 3.04 (s, 2H), 3.23 (m, 2H), 3.66 (m, 2H), 7.44 (d, 1H), 7.67 (d, 1H), 8.12 (s, 1H).

### Step 5: Synthesis of 5-bromo-2-((4-methylpiperidin-4-yl)methyl)benzo[d]thiazole

To a solution of tert-butyl 4-[(5-bromo-1,3-benzothiazol-2-yl)methyl]-4-methyl-piperidine-1-carboxylate (5.4 g, 12.69 mmol) in Et₂O (20.00 mL) was added hydrogen chloride solution 4.0M in dioxane (24.00 g, 658.24 mmol, 30.00 mL) at 21° C. The resulting mixture was left to stir for 6 hr. The resulting mixture was evaporated to dryness and was used in the next step without further purification. 5-Bromo-2-[(4-methyl-4-piperidyl)methyl]-1,3-benzothiazole (4.5 g, crude, 2HCl) was obtained as a beige solid. LCMS(ESI): [M]⁺ m/z: calcd 325.2; found 326.2; Rt=2.325 min.

### Step 6: Synthesis of 5-bromo-2-((1,4-dimethylpiperidin-4-yl)methyl)benzo[d]thiazole

Formaldehyde, 37% w/w aq. soln., stabilized with 7-8% MeOH (1.01 g, 12.43 mmol, 931.63 μL, 37% purity) and acetic acid (2.04 g, 33.90 mmol, 1.94 mL) were added to the solution of 5-bromo-2-[(4-methyl-4-piperidyl)methyl]-1,3-benzothiazole (4.5 g, 11.30 mmol, 2HCl) and sodium acetate, anhydrous (1.85 g, 22.60 mmol, 1.21 mL) in MeOH. The resulting mixture was stirred at 21° C. for 1 hr before sodium cyan borohydride (1.42 g, 22.60 mmol) was added thereto. After that, stirring was continued for 8 hr. Then, the solvent was removed under reduced pressure, and residue was partitioned between 10% aq. NaOH solution (20 ml) and DCM (40 ml). The organic layer was separated, evaporated. 5-bromo-2-[(1,4-dimethyl-4-piperidyl)methyl]-1,3-benzothiazole (4.3 g, crude) was obtained as a yellow gum. LCMS(ESI): [M]⁺ m/z: calcd 339.2; found 340.2; Rt=2.474 min.

### Step 7: Synthesis of 2-((1,4-dimethylpiperidin-4-yl)methyl)-5-(4,4,5,5-tetramethyl-1,3,2-dioxaborolan-2-yl)benzo[d]thiazole

The synthesis of 2-((1,4-dimethylpiperidin-4-yl)methyl)-5-(4,4,5,5-tetramethyl-1,3,2-dioxaborolan-2-yl)benzo[d]thiazole is given by General Scheme S2, Step 2. Yield: 4.5 g of crude.

LCMS(ESI): [M]⁺ m/z: calcd 386.2; found 387.2; Rt=2.490 min.

### Step 8: Synthesis of(S)-tert-butyl 6-(2-((1,4-dimethylpiperidin-4-yl)methyl)benzo[d]thiazol-5-yl)-3-methyl-3,4-dihydropyridine-1(2H)-carboxylate

The synthesis of(S)-tert-butyl 6-(2-((1,4-dimethylpiperidin-4-yl)methyl)benzo[d]thiazol-5-yl)-3-methyl-3,4-dihydropyridine-1(2H)-carboxylate is given by General Scheme S2, Step 3. Yield: 7.5 g of crude. LCMS(ESI): [M]⁺ m/z: calcd 455.2; found 456.2; Rt=3.572 min.

### Step 9: Synthesis of(S)-2-((1,4-dimethylpiperidin-4-yl)methyl)-5-(5-methyl-3,4,5,6-tetrahydropyridin-2-yl)benzo[d]thiazole

To a solution of tert-butyl (3S)-6-[2-[(1,4-dimethyl-4-piperidyl)methyl]-1,3-benzothiazol-5-yl]-3-methyl-3,4-dihydro-2H-pyridine-1-carboxylate (7.5 g, 16.46 mmol) in DCM (18.66 mL) was added TFA (9.38 g, 82.30 mmol, 6.34 mL) in one portion and the resulting mixture was left to stir overnight at rt. The reaction mixture was quenched with aq NaOH (30%). Water was extracted with DCM (3×50 mL). Combined organics were dried over Na₂SO₄ and evaporated to give a residue which was used in the next step without further purification. LCMS(ESI): [M]⁺ m/z: calcd 355.2; found 356.2; Rt=1.424 min.

### Step 10: Synthesis of 2-((1,4-dimethylpiperidin-4-yl)methyl)-5-((2R,5S)-5-methylpiperidin-2-yl)benzo[d]thiazole

The synthesis of 2-((1,4-dimethylpiperidin-4-yl)methyl)-5-((2R,5S)-5-methylpiperidin-2-yl)benzo[d]thiazole is given by General Scheme S2, step 5. Yield: 0.77 g (15.31%). LCMS(ESI): [M]⁺ m/z: calcd 357.2; found 358.2; Rt=0.665 min.

### Step 11: Synthesis of 2-((2R,5S)-2-(2-((1,4-dimethylpiperidin-4-yl)methyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

2-[(1,4-Dimethyl-4-piperidyl)methyl]-5-[(2R,5S)-5-methyl-2-piperidyl]-1,3-benzothiazole (0.1 g, 279.68 μmol), HATU (106.34 mg, 279.68 μmol) and TEA (56.60 mg, 559.35 μmol, 77.96 μL) were mixed in dry DMF (2 mL) at rt and the resulting mixture was stirred for 15 min. 2-Oxo-2-[[1-(2-trimethylsilylethoxymethyl)pyrazolo[4,3-c]pyridin-7-yl]amino]acetic acid (94.09 mg, 279.68 μmol) was added thereto and the resulting mixture was stirred at rt overnight. The resulting mixture was poured into water, extracted 3 times with EtOAc, combined organics were washed with water, brine and evaporated. 2-Oxo-2-[(2R,5S)-2-[2-[(1,4-dimethyl-4-piperidyl)methyl]-1,3-benzothiazol-5-yl]-5-methyl-1-piperidyl]-N-[1-(2-trimethylsilylethoxymethyl)pyrazolo[4,3-c]pyridin-7-yl]acetamide (0.15 g, 221.91 μmol, 79.34% yield) was used in the next step without further purification. LCMS(ESI): [M]⁺ m/z: calcd 675.2; found 676.2; Rt=3.042 min.

### Step 12: Synthesis of 2-((2R,5S)-2-(2-((1,4-dimethylpiperidin-4-yl)methyl)benzo[d]thiazol-5-yl)-5-methylpiperidin-1-yl)-2-oxo-N-(1H-pyrazolo[4,3-c]pyridin-7-yl)acetamide (Compound 142)

To a solution of 2-oxo-2-[(2R,5S)-2-[2-[(1,4-dimethyl-4-piperidyl)methyl]-1,3-benzothiazol-5-yl]-5-methyl-1-piperidyl]-N-[1-(2-trimethylsilylethoxymethyl)pyrazolo[4,3-c]pyridin-7-yl]acetamide (0.15 g, 221.91 μmol) in MeOH (4.90 mL) was added hydrogen chloride solution 4.0M in dioxane (4.00 g, 109.71 mmol, 5 mL) at 21° C. The resulting mixture was left to stir for 2 hr. The resulting mixture was evaporated to dryness and subjected to HPLC (20-45% water-MeCN+NH₃, 30 ml/min). 2-Oxo-N-(1H-pyrazolo[4,3-c]pyridin-7-yl)-2-[(2R,5S)-2-[2-[(1,4-dimethyl-4-piperidyl)methyl]-1,3-benzothiazol-5-yl]-5-methyl-1-piperidyl]acetamide (38.5 mg, 70.55 μmol, 31.79% yield) was obtained as a brown solid.

Compound 142: ¹H NMR (600 MHz, DMSO-d₆) δ (ppm) 0.75-0.99 (m, 3H), 1.00-1.10 (m, 3H), 1.32-1.44 (m, 3H), 1.56-1.64 (m, 2H), 1.70-1.81 (m, 1H), 1.86-1.98 (m, 1H), 2.16 (s, 3H), 2.20-2.27 (m, 2H), 2.30-2.37 (m, 1H), 2.39-2.47 (m, 3H), 2.87-2.93 (m, 0.3H), 2.99-3.09 (m, 2H), 3.36-3.41 (m, 0.7H), 3.72-4.38 (m, 1H), 5.56-5.88 (m, 1H), 7.38-7.46 (m, 1H), 7.89-7.98 (m, 1H), 8.00-8.11 (m, 1H), 8.28-8.39 (m, 1H), 8.41-8.56 (m, 1H), 8.86-9.01 (m, 1H), 10.76-11.38 (m, 1H), 12.80-13.36 (m, 1H).

LCMS(ESI): [M]⁺ m/z: calcd 545.2; found 546.2; Rt=0.936 min.

### Example 109. Synthesis of 2-((2R,5S)-2-(2-(1-azabicyclo[2.2.1]heptan-3-yl)benzo[d]thiazol-5-yl)-5-methylpiperidin-1-yl)-2-oxo-N-(1H-pyrazolo[4,3-c]pyridin-7-yl)acetamide (Compound 134)

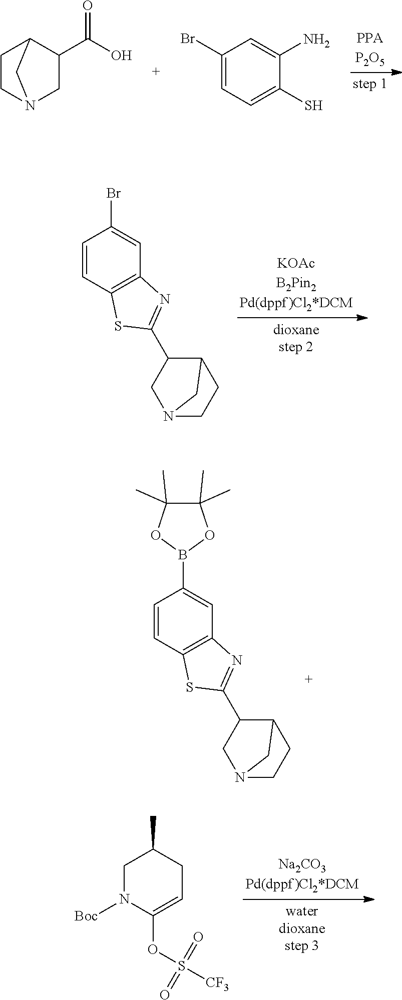 [SMILES: O=C(O)C1CN2CCC1C2 ; Nc1cc(Br)ccc1S ; Brc1ccc2sc(C3CN4CCC3C4)nc2c1 ; CC1(C)OB(c2ccc3sc(C4CN5CCC4C5)nc3c2)OC1(C)C ; C[C@H]1CC=C(OS(=O)(=O)C(F)(F)F)N(C(=O)OC(C)(C)C)C1]

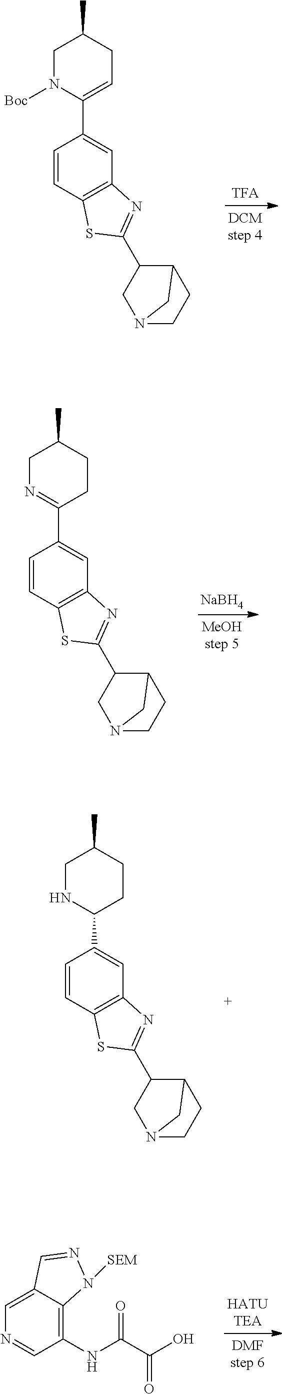 [SMILES: C[C@H]1CC=C(c2ccc3sc(C4CN5CCC4C5)nc3c2)N(C(=O)OC(C)(C)C)C1 ; C[C@H]1CCC(c2ccc3sc(C4CN5CCC4C5)nc3c2)=NC1 ; "NaBH4" ; C[C@H]1CC[C@H](c2ccc3sc(C4CN5CCC4C5)nc3c2)NC1 ; C[Si](C)(C)CCOCn1ncc2cncc(NC(=O)C(=O)O)c21]

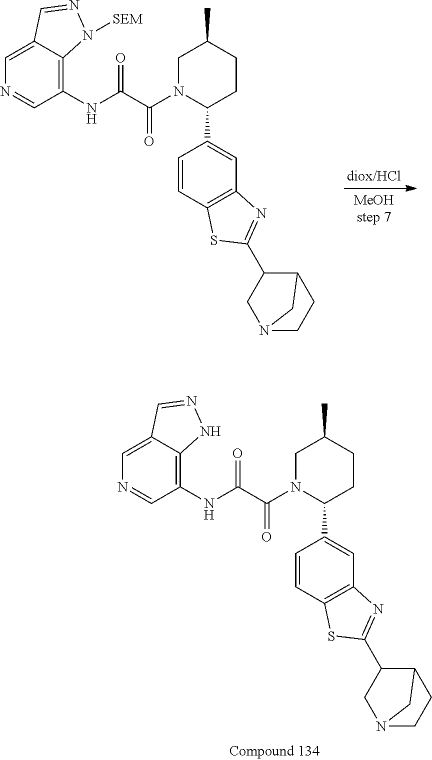 [SMILES: C[C@H]1CC[C@H](c2ccc3sc(C4CN5CCC4C5)nc3c2)N(C(=O)C(=O)Nc2cncc3cnn(COCC[Si](C)(C)C)c23)C1 ; C[C@H]1CC[C@H](c2ccc3sc(C4CN5CCC4C5)nc3c2)N(C(=O)C(=O)Nc2cncc3cn[nH]c23)C1]

### Step 1: Synthesis of 2-(1-azabicyclo[2.2.1]heptan-3-yl)-5-bromobenzo[d]thiazole

The Synthesis of 2-(1-azabicyclo[2.2.1]heptan-3-yl)-5-bromobenzo[d]thiazole is given by General Scheme S2, Step 1A. Yield: 1.4 g of crude. LCMS(ESI): [M]⁺ m/z: calcd 308.2; found 309.2; Rt=0.915 min.

### Step 2: Synthesis of 2-(1-azabicyclo[2.2.1]heptan-3-yl)-5-(4,4,5,5-tetramethyl-1,3,2-dioxaborolan-2-yl)benzo[d]thiazole

The synthesis of 2-(1-azabicyclo[2.2.1]heptan-3-yl)-5-(4,4,5,5-tetramethyl-1,3,2-dioxaborolan-2-yl)benzo[d]thiazole is given by General Scheme S2, Step 2. Yield: 1.8 g of crude. LCMS(ESI): [M]⁺ m/z: calcd 356.2; found 357.2; Rt=1.138 min.

### Step 3: Synthesis of tert-butyl (3S)-6-(2-(1-azabicyclo[2.2.1]heptan-3-yl)benzo[d]thiazol-5-yl)-3-methyl-3,4-dihydropyridine-1(2H)-carboxylate

The synthesis of tert-butyl (3S)-6-(2-(1-azabicyclo[2.2.1]heptan-3-yl)benzo[d]thiazol-5-yl)-3-methyl-3,4-dihydropyridine-1(2H)-carboxylate is given by General Scheme S2, Step 3. Yield: 1.4 g of crude. LCMS(ESI): [M]⁺ m/z: calcd 425.2; found 426.2; Rt=0.920 min.

### Step 4: Synthesis of 2-(1-azabicyclo[2.2.1]heptan-3-yl)-5-((S)-5-methyl-3,4,5,6-tetrahydropyridin-2-yl)benzo[d]thiazole

The synthesis of 2-(1-azabicyclo[2.2.1]heptan-3-yl)-5-((S)-5-methyl-3,4,5,6-tetrahydropyridin-2-yl)benzo[d]thiazole is given by General Scheme S2, Step 4. Yield: 0.6 g of crude. LCMS(ESI): [M]⁺ m/z: calcd 325.2; found 326.2; Rt=0.534 min.

### Step 5: Synthesis of 2-(1-azabicyclo[2.2.1]heptan-3-yl)-5-((2R,5S)-5-methylpiperidin-2-yl)benzo[d]thiazole

The synthesis of 2-(1-azabicyclo[2.2.1]heptan-3-yl)-5-((2R,5S)-5-methylpiperidin-2-yl)benzo[d]thiazole is given by General Scheme S2, Step 5. Yield: 0.6 g of crude. LCMS(ESI): [M]⁺ m/z: calcd 327.2; found 328.2; Rt=0.700 min.

### Step 6: Synthesis of 2-((2R,5S)-2-(2-(1-azabicyclo[2.2.1]heptan-3-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

The synthesis of 2-((2R,5S)-2-(2-(1-azabicyclo[2.2.1]heptan-3-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide is given by General Scheme S2, Step 6A. Yield: 21.8 mg of crude. HPLC conditions: Column: Chromatorex C18 100*19 mm, 5 microM; 0-5 min 50-85% water-MeOH, flow: 30 ml/min; (loading pump 4 ml/min MeOH). LCMS(ESI): [M]⁺ m/z: calcd 645.2; found 646.2; Rt=1.227 min.

### Step 7: Synthesis of 2-((2R,5S)-2-(2-(1-azabicyclo[2.2.1]heptan-3-yl)benzo[d]thiazol-5-yl)-5-methylpiperidin-1-yl)-2-oxo-N-(1H-pyrazolo[4,3-c]pyridin-7-yl)acetamide (Compound 134)

2-((2R,5S)-2-(2-(1-Azabicyclo[2.2.1]heptan-3-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide (21.8 mg, 33.65 μmol) was dissolved in a mixture of MeOH (1 mL) and diox/HCl (1 mL), the resulting mixture was stirred at 25° C. for 17 hr. After that time, the solvent was evaporated to dryness to afford crude product, then crude product was subjected by HPLC (column: XBridge BEH C18, 5 um 130 A; mobile phase: 20-85% 0-5 min H₂O/MeOH/0.1% NH₄OH, flow rate: 30 ml/min (loading pump 4 ml/min MeOH), affording 2-((2R,5S)-2-(2-(1-azabicyclo[2.2.1]heptan-3-yl)benzo[d]thiazol-5-yl)-5-methylpiperidin-1-yl)-2-oxo-N-(1H-pyrazolo[4,3-c]pyridin-7-yl)acetamide (8.2 mg, 15.84 μmol, 47.08% yield).

Compound 134: ¹H NMR (600 MHZ, DMSO-d₆) δ (ppm) 1.04-1.09 (m, 3H), 1.23-1.62 (m, 3H), 1.68-1.82 (m, 2H), 1.86-1.97 (m, 1H), 2.11-2.37 (m, 3H), 2.67-3.20 (m, 7H), 3.75-4.16 (m, 1H), 5.54-5.83 (m, 1H), 7.37-7.48 (m, 1H), 7.88-7.96 (m, 1H), 8.00-8.12 (m, 1H), 8.30-8.39 (m, 1H), 8.39-8.56 (m, 1H), 8.88-9.00 (m, 1H), 11.02-11.24 (m, 1H), 12.92-13.18 (m, 1H). LCMS(ESI): [M]⁺ m/z: calcd 515.2; found 516.2; Rt=2.114 min.

### Example 110. Synthesis of 2-oxo-N-(1H-pyrazolo[4,3-c]pyridin-7-yl)-2-[rac-(5S)-5-methyl-2-[2-[1-(trideuteriomethyl)-4-piperidyl]-1,3-benzothiazol-5-yl]-1-piperidyl]acetamide (Compound 119)

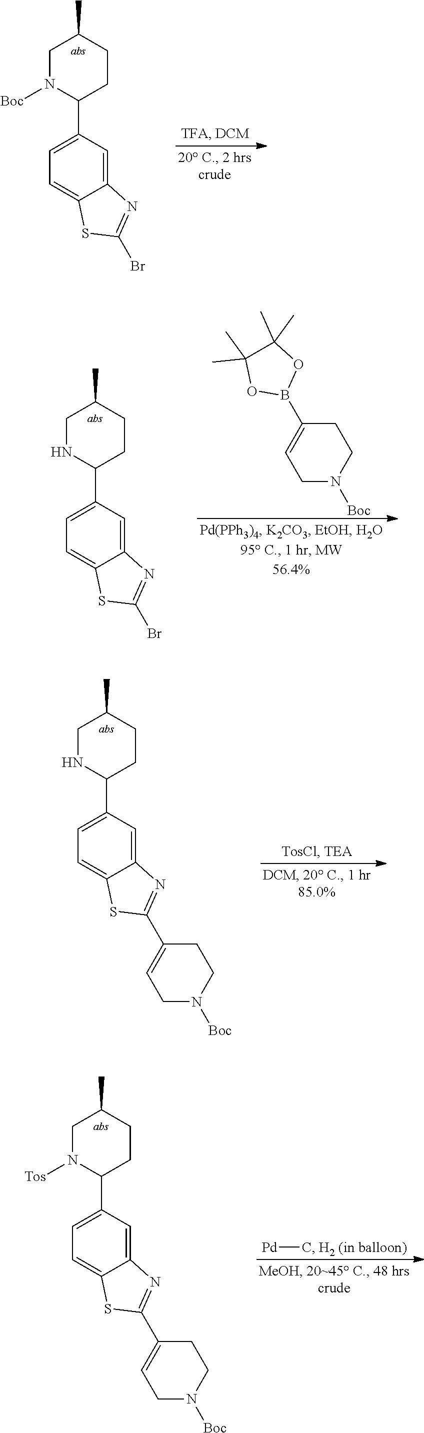

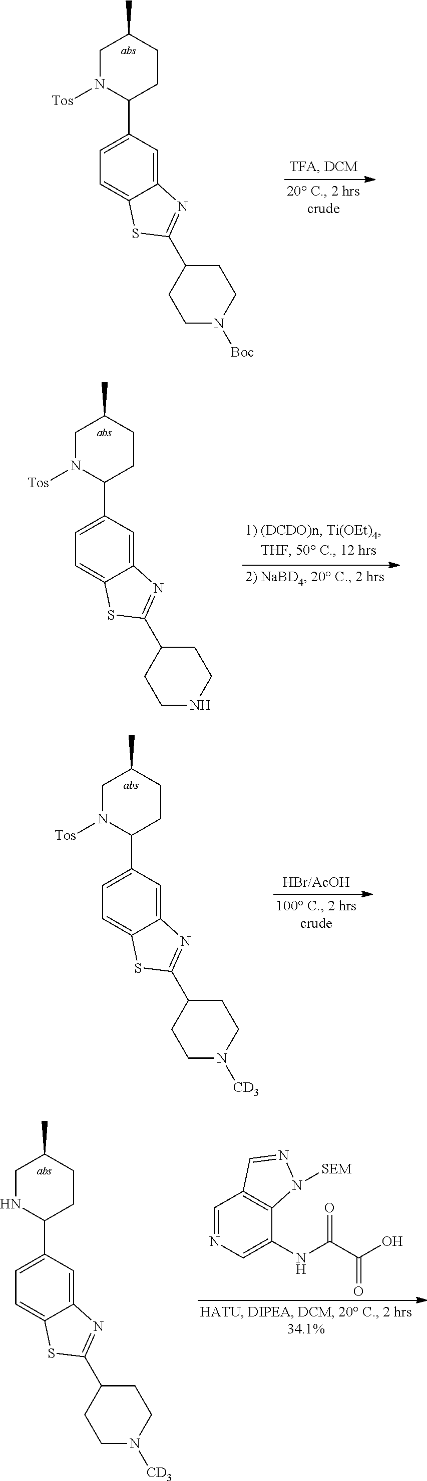

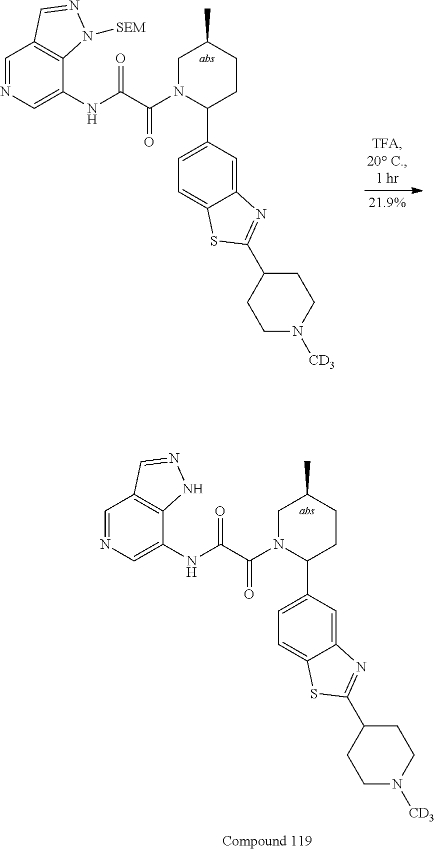 [SMILES: [2H]C([2H])([2H])N1CCC(c2nc3cc(C4CC[C@H](C)CN4C(=O)C(=O)Nc4cncc5cnn(COCC[Si](C)(C)C)c45)ccc3s2)CC1 ; [2H]C([2H])([2H])N1CCC(c2nc3cc(C4CC[C@H](C)CN4C(=O)C(=O)Nc4cncc5cn[nH]c45)ccc3s2)CC1]

### Step 1: Synthesis of 2-bromo-5-[(5S)-5-methyl-2-piperidyl]-1,3-benzothiazole

A mixture of tert-butyl (5S)-2-(2-bromo-1,3-benzothiazol-5-yl)-5-methyl-piperidine-1-carboxylate (500 mg, 1.22 mmol), DCM (3 mL) and TFA (1.50 mL, 19.5 mmol) was stirred at 20° C. for 2 hours. The resulting mixture was adjusted to pH=9 with saturated K₂CO₃ aqueous solution, and then the mixture was extracted with DCM (100 mL*3). The combined organic layer was washed with brine (100 mL), dried over anhydrous Na₂SO₄, filtered and concentrated under reduced pressure to give 2-bromo-5-[(5S)-5-methyl-2-piperidyl]-1,3-benzothiazole (400 mg, crude) as a yellow solid. LCMS(ESI) [M+H]⁺ m/z: calcd 313.0, found 313.0.

### Step 2: Synthesis of tert-butyl 4-[5-[rac-(5S)-5-methyl-2-piperidyl]-1,3-benzothiazol-2-yl]-3,6-dihydro-2H-pyridine-1-carboxylate

To a mixture of 2-bromo-5-[rac-(5S)-5-methyl-2-piperidyl]-1,3-benzothiazole (400 mg, 1.29 mmol) and tert-butyl 4-(4,4,5,5-tetramethyl-1,3,2-dioxaborolan-2-yl)-3,6-dihydro-2H-pyridine-1-carboxylate (480 mg, 1.55 mmol) in EtOH (4 mL) and H₂O (2 mL) were added Pd(PPh₃)₄ (160 mg, 0.138 mmol) and K₂CO₃ (520 mg, 3.76 mmol). The resulting mixture was stirred at 95° C. for 1 hour under microwave. The resulting mixture was concentrated under reduced pressure to give the residue, which was purified by flash chromatography (ISCO®; 12 g AgelaFlash® Silica Flash Column, DCM/MeOH with MeOH from 0˜13%, flow rate=30 mL/min, 254 nm) to afford tert-butyl 4-[5-[rac-(5S)-5-methyl-2-piperidyl]-1,3-benzothiazol-2-yl]-3,6-dihydro-2H-pyridine-1-carboxylate (300 mg, 56.4% yield) as brown oil. LCMS(ESI) [M+H]⁺ m/z: calcd 414.2, found 414.2.

### Step 3: Synthesis of tert-butyl 4-[5-[rac-(5S)-5-methyl-1-(p-tolylsulfonyl)-2-piperidyl]-1,3-benzothiazol-2-yl]-3,6-dihydro-2H-pyridine-1-carboxylate

To a mixture of tert-butyl 4-[5-[rac-(5S)-5-methyl-2-piperidyl]-1,3-benzothiazol-2-yl]-3,6-dihydro-2H-pyridine-1-carboxylate (300 mg, 0.725 mmol) in DCM (5 mL) were added TEA (2.15 mmol, 0.3 mL) and 4-methylbenzenesulfonyl chloride (165 mg, 0.865 mmol) slowly. The resulting mixture was stirred at 20° C. for 1 hour. The resulting mixture was quenched by addition of water (50 mL) and extracted with EtOAc (100 mL*3). The combined organic layer was washed with saturated NH₄Cl aqueous solution (50 mL), brine (100 mL), dried over anhydrous Na₂SO₄, filtered and concentrated under reduced pressure. The residue was purified by flash chromatography (ISCO®; 12 g AgelaFlash® Silica Flash Column, petroleum ether/EtOAc with EtOAc from 0˜25%, flow rate=30 mL/min, 254 nm) to afford tert-butyl 4-[5-[rac-(5S)-5-methyl-1-(p-tolylsulfonyl)-2-piperidyl]-1,3-benzothiazol-2-yl]-3,6-dihydro-2H-pyridine-1-carboxylate (350 mg, 85.0% yield) as a colorless oil.

LCMS(ESI) [M+H]⁺ m/z: calcd 568.2, found 568.2.

### Step 4: Synthesis of tert-butyl 4-[5-[rac-(5S)-5-methyl-1-(p-tolylsulfonyl)-2-piperidyl]-1,3-benzothiazol-2-yl]piperidine-1-carboxylate

A mixture of tert-butyl 4-[5-[rac-(5S)-5-methyl-1-(p-tolylsulfonyl)-2-piperidyl]-1,3-benzothiazol-2-yl]-3,6-dihydro-2H-pyridine-1-carboxylate (300 mg, 0.528 mmol) and Pd/C (60 mg, 10 wt % Pd with 50 wt % water) in MeOH (5 mL) was stirred at 20° C. for 12 hours under hydrogen (in balloon). The resulting mixture was filtered and concentrated under reduced pressure to give the residue, which was diluted with MeOH (5 mL) and added Pd/C (60 mg, 10 wt % Pd with 50 wt % water). The mixture was stirred at 45° C. for 36 hours under hydrogen (in balloon). The resulting mixture was filtered and concentrated under reduced pressure to give tert-butyl 4-[5-[rac-(5S)-5-methyl-1-(p-tolylsulfonyl)-2-piperidyl]-1,3-benzothiazol-2-yl]piperidine-1-carboxylate (200 mg, crude) as a yellow oil. LCMS(ESI) [M+H]⁺ m/z: calcd 568.2, found 568.2.

### Step 5: Synthesis of 2-(4-piperidyl)-5-[rac-(5S)-5-methyl-1-(p-tolylsulfonyl)-2-piperidyl]-1,3-benzothiazole

A mixture of tert-butyl 4-[5-[rac-(5S)-5-methyl-1-(p-tolylsulfonyl)-2-piperidyl]-1,3-benzothiazol-2-yl]piperidine-1-carboxylate (200 mg, 0.351 mmol), DCM (5 mL) and TFA (0.5 mL, 6.49 mmol) was stirred at 20° C. for 2 hours. The resulting mixture was concentrated under reduced pressure to give 2-(4-piperidyl)-5-[rac-(5S)-5-methyl-1-(p-tolylsulfonyl)-2-piperidyl]-1,3-benzothiazole (160 mg, crude, TFA) as a yellow oil, which was directly used in the next step without further purification. LCMS(ESI) [M+H]⁺ m/z: calcd 470.2, found 470.2.

### Step 6: Synthesis of 5-[rac-(5S)-5-methyl-1-(p-tolylsulfonyl)-2-piperidyl]-2-[1-(trideuteriomethyl)-4-piperidyl]-1,3-benzothiazole

A mixture of 2-(4-piperidyl)-5-[rac-(5S)-5-methyl-1-(p-tolylsulfonyl)-2-piperidyl]-1,3-benzothiazole (160 mg, 0.275 mmol, TFA) in THF (5 mL) was adjusted to pH=8 with Na₂CO₃, then AcOH (0.275 mmol, 0.05 mL), (DCDO)ₙ (48 mg, 1.37 mmol) and Ti(OEt)₄ (260 mg, 1.14 mmol) were added to the mixture. The resulting mixture was stirred at 50° C. for 12 hours. NaBD₄ (20 mg, 0.502 mmol) was added. The mixture was stirred at 20° C. for 2 hours. The resulting mixture was quenched by addition of water (20 mL) and extracted with EtOAc (100 mL*3). The combined organic layer was washed with brine (50 mL), dried over anhydrous Na₂SO₄, filtered and concentrated under reduced pressure. The residue was purified by flash chromatography (ISCO®; 4 g AgelaFlash® Silica Flash Column, DCM/MeOH with MeOH from 0˜13%, flow rate=30 mL/min, 254 nm) to afford 5-[rac-(5S)-5-methyl-1-(p-tolylsulfonyl)-2-piperidyl]-2-[1-(trideuteriomethyl)-4-piperidyl]-1,3-benzothiazole (90 mg, 67.3% yield) as white solid. LCMS(ESI) [M+H]⁺ m/z: calcd 487.2, found 487.3.

### Step 7: Synthesis of 5-[rac-(5S)-5-methyl-2-piperidyl]-2-[1-(trideuteriomethyl)-4-piperidyl]-1,3-benzothiazole

A mixture of 5-[rac-(5S)-5-methyl-1-(p-tolylsulfonyl)-2-piperidyl]-2-[1-(trideuteriomethyl)-4-piperidyl]-1,3-benzothiazole (80 mg, 0.164 mmol), HBr/AcOH (1 mL, 0.164 mmol) was stirred at 100° C. for 2 hours. The resulting mixture was concentrated under reduced pressure to remove HBr/AcOH and adjusted to pH=9 with saturated Na₂CO₃ aqueous solution. The solution was stirred at 25° C. for 12 hours. The resulting mixture was filtered and concentrated under reduced pressure to afford 5-[rac-(5S)-5-methyl-2-piperidyl]-2-[1-(trideuteriomethyl)-4-piperidyl]-1,3-benzothiazole (60 mg, crude) as a yellow solid.

LCMS(ESI) [M+H]⁺ m/z: calcd 333.2, found 333.1.

### Step 8: Synthesis of 2-oxo-2-[rac-(5S)-5-methyl-2-[2-[1-(trideuteriomethyl)-4-piperidyl]-1,3-benzothiazol-5-yl]-1-piperidyl]-N-[1-(2-trimethylsilylethoxymethyl)pyrazolo[4,3-c]pyridin-7-yl]acetamide

A mixture of 5-[rac-(5S)-5-methyl-2-piperidyl]-2-[1-(trideuteriomethyl)-4-piperidyl]-1,3-benzothiazole (60.0 mg, 0.180 mmol), 2-oxo-2-[[1-(2-trimethylsilylethoxymethyl)pyrazolo[4,3-c]pyridin-7-yl]amino]acetic acid (60.0 mg, 0.178 mmol), HATU (80 mg, 0.210 mmol), DIPEA (0.090 mL, 0.517 mmol) in DMF (2 mL) was stirred at 20° C. for 2 hours. The resulting mixture was concentrated under reduced pressure to give a residue, which was purified by flash chromatography (ISCO®; 4 g AgelaFlash® Silica Flash Column, petroleum ether/EtOAc with EtOAc from 0˜50%, flow rate=30 mL/min, 254 nm) to 2-oxo-2-[rac-(5S)-5-methyl-2-[2-[1-(trideuteriomethyl)-4-piperidyl]-1,3-benzothiazol-5-yl]-1-piperidyl]-N-[1-(2-trimethylsilylethoxymethyl)pyrazolo[4,3-c]pyridin-7-yl]acetamide (40 mg, 34.1% yield) as yellow oil. LCMS(ESI) [M+H]⁺ m/z: calcd 651.3, found 651.4.

### Step 9: Synthesis of 2-oxo-N-(1H-pyrazolo[4,3-c]pyridin-7-yl)-2-[rac-(5S)-5-methyl-2-[2-[1-(trideuteriomethyl)-4-piperidyl]-1,3-benzothiazol-5-yl]-1-piperidyl]acetamide (Compound 119)

A mixture of 2-oxo-2-[rac-(5S)-5-methyl-2-[2-[1-(trideuteriomethyl)-4-piperidyl]-1,3-benzothiazol-5-yl]-1-piperidyl]-N-[1-(2-trimethylsilylethoxymethyl)pyrazolo[4,3-c]pyridin-7-yl]acetamide (40 mg, 61.5 μmol), TFA (1 mL, 13.0 mmol) was stirred at 20° C. for 1 hour. The mixture was concentrated under reduced pressure to give a crude product, which was purified by preparative HPLC (Instrument: Gilson GX-281 Liquid Handler, Gilson 322 Pump, Gilson 156 UV Detector; Column: 2_Phenomenex Gemini C18 75*40 mm*3 μm; Mobile phase A: H₂O with 0.05% NH₄HCO₃ (v %); Mobile phase B: MeCN; Gradient: B from 30% to 60% in 7.8 min, hold 100% B for 2 min; Flow Rate: 25 mL/min; Column Temperature: 30° C.; Wavelength: 220 nm, 254 nm) to afford 2-oxo-N-(1H-pyrazolo[4,3-c]pyridin-7-yl)-2-[rac-(5S)-5-methyl-2-[2-[1-(trideuteriomethyl)-4-piperidyl]-1,3-benzothiazol-5-yl]-1-piperidyl]acetamide (7 mg, 21.9% yield) as a white solid. ¹H NMR (400 MHz, methanol-d₄) δ ppm 8.86-9.08 (m, 1H), 8.22-8.58 (m, 2H), 7.88-8.12 (m, 2H), 7.49 (br s, 1H), 5.66-5.92 (m, 1H), 3.45-4.16 (m, 2H), 3.09 (br d, J=10.8 Hz, 2H), 2.37 (br s, 4H), 2.23 (br s, 2H), 2.02 (br d, J=18.6 Hz, 5H), 1.50 (br d, J=13.1 Hz, 1H), 1.18 (br d, J=6.8 Hz, ³H); LCMS(ESI) [M+H]⁺ m/z: calcd 521.2, found 521.2; HPLC: 100%@254 nm; SFC: 85.6% de.

### Example 111. Synthesis of N-(4-amino-1H-pyrazolo[4,3-c]pyridin-7-yl)-2-oxo-2-[(2R,5S)-5-methyl-2-phenyl-1-piperidyl]acetamide (Compound 126)

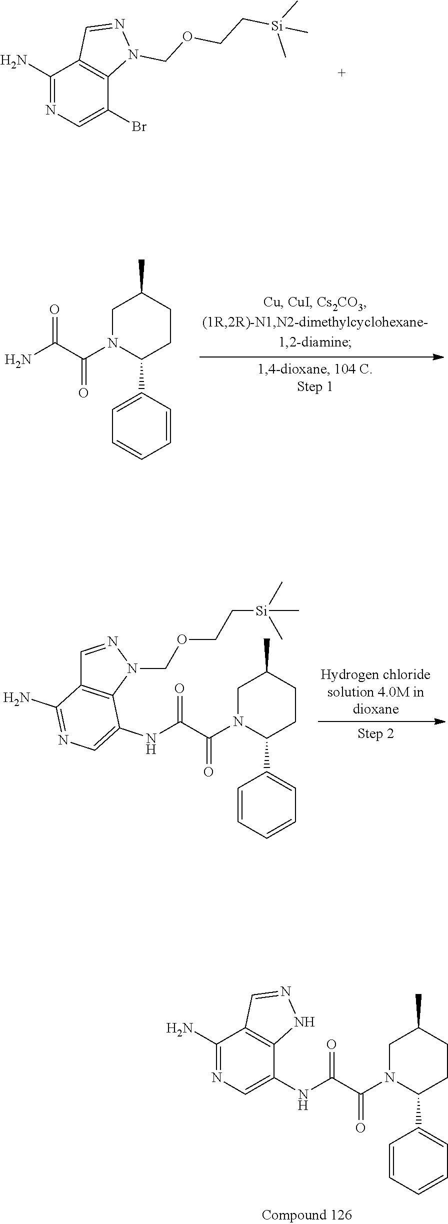

### Step 1: The Synthesis of N-[4-amino-1-(2-trimethylsilylethoxymethyl)pyrazolo[4,3-c]pyridin-7-yl]-2-oxo-2-[(2R,5S)-5-methyl-2-phenyl-1-piperidyl]acetamide

A mixture of 2-oxo-2-[(2R,5S)-5-methyl-2-phenyl-1-piperidyl]acetamide (350 mg, 1.42 mmol), 7-bromo-1-(2-trimethylsilylethoxymethyl)pyrazolo[4,3-c]pyridin-4-amine (536.61 mg, 1.56 mmol), copper* (5.37 mg, 84.43 μmol), Copper (I) iodide (140 mg, 735.10 μmol, 24.91 μL), cesium carbonate (694.49 mg, 2.13 mmol) and (1R,2R)—N1,N2-dimethylcyclohexane-1,2-diamine (140 mg, 984.25 μmol) in 1,4-dioxane** (6.5 mL) was stirred in a sealed vial under argon at 105° C. for 42 hr***. The resulting mixture was cooled down, diluted with DMSO (1 ml)****, and submitted to reverse phase HPLC (column: XBridge C18 100×19 mm, 5 um; mobile phase: 50-100% 0-5 min H₂O/MeOH/0.1% NH₄OH; flow rate: 30 ml/min (loading pump 4 ml/min methanol) to afford N-[4-amino-1-(2-trimethylsilylethoxymethyl)pyrazolo[4,3-c]pyridin-7-yl]-2-oxo-2-[(2R,5S)-5-methyl-2-phenyl-1-piperidyl]acetamide (491 mg, 965.23 μmol, 67.93% yield) as light-brown gum.

LCMS(ESI): [M+H]⁺ m/z: calcd 508.3; found 509.2; Rt=1.049 min. *copper was prepared by the following procedure: Zn powder 2 eq was added slowly under intensive stirring to 10% solution of Cu(NO3)2 (10 eq) in water. The mixture was stirred for 90 min, then the resulting solid was filtered, washed with 10% solution of Cu(NO3)2, and distilled water. The solids were dried for 5 hr at 80° C. to give black powder of copper that used for Ullmann reaction**1,4-dioxane was dried over molecular sieves***the reaction requires 42 hr. An aliquot after 24 hr showed remaining starting materials. Additional heating after taking the aliquot didn't improve conversion, thus the recommendation is to perform the reaction over 42 hr without stopping**** the insoluble materials must be removed with centrifuge before HPLC

### Step 2: The Synthesis of N-(4-amino-1H-pyrazolo[4,3-c]pyridin-7-yl)-2-oxo-2-[(2R,5S)-5-methyl-2-phenyl-1-piperidyl]acetamide (Compound 126)

Hydrogen chloride solution 4.0M in dioxane (8.40 g, 32.02 mmol, 8.00 mL, 13.9% purity) was added to a stirred solution of N-[4-amino-1-(2-trimethylsilylethoxymethyl)pyrazolo[4,3-c]pyridin-7-yl]-2-oxo-2-[(2R,5S)-5-methyl-2-phenyl-1-piperidyl]acetamide (1.10 g, 2.16 mmol) in methanol (8.00 mL) at 25° C. The resulting solution was stirred at 25° C. for 20 hr, then concentrated to dryness in vacuo and the residue was submitted to reverse phase HPLC (column: Chromatorex 18 SMB100-5T 100×19 mm 5 um; mobile phase: 10-25% 0-5 min H₂O/ACN/FA, flow: 30 ml/min (loading pump 4 ml/min acetonitrile)) to afford Compound 126 N-(4-amino-1H-pyrazolo[4,3-c]pyridin-7-yl)-2-oxo-2-[(2R,5S)-5-methyl-2-phenyl-1-piperidyl]acetamide (615 mg, 1.45 mmol, 67.19% yield, HCOOH) as light-yellow solid. LCMS(ESI): [M+H]⁺ m/z: calcd 378.2; found 379.4; Rt=2.471 min.

### Example 112. Synthesis of 2-((2R,5S)-2-(2-(1,5-dimethyl-1,2,3,6-tetrahydropyridin-4-yl)benzo[d]thiazol-5-yl)-5-methylpiperidin-1-yl)-N-(imidazo[1,2-a]pyridin-7-yl)-2-oxoacetamide (Compound 125)

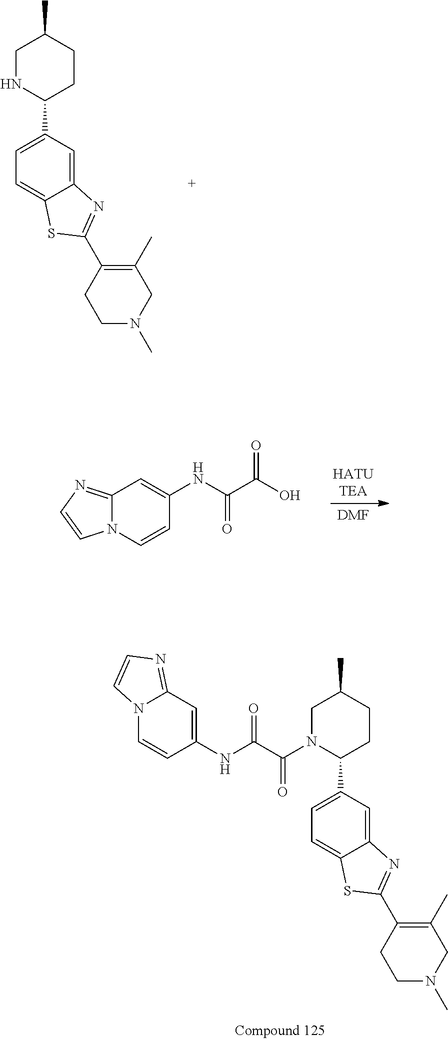

Synthesis of 2-(1,5-dimethyl-1,2,3,6-tetrahydropyridin-4-yl)-5-((2R,5S)-5-methylpiperidin-2-yl)benzo[d]thiazole is given by General Scheme S2, Step 5.

HPLC conditions: Column: XBridge C18 100*19 mm, 5 microM; 0-5 min 40-90% water-MeOH+0.1% NH₄OH; (loading pump 4 ml/min MeOH).

Compound 125: ¹H NMR (500 MHz, DMSO-d₆) δ (ppm) 0.80-1.10 (m, 3H), 1.30-1.49 (m, 1H), 1.69-1.81 (m, 1H), 1.84-1.97 (m, 1H), 2.07-2.13 (m, 3H), 2.13-2.27 (m, 1H), 2.27-2.35 (m, 3H), 2.51-2.59 (m, 3H), 2.62-2.68 (m, 2H), 2.88-2.92 (m, 0.3H), 2.97-3.10 (m, 2H), 3.21-3.26 (m, 0.7H), 3.53-4.06 (m, 1H), 5.25-5.84 (m, 1H), 6.97-7.15 (m, 1H), 7.35-7.44 (m, 1H), 7.44-7.51 (m, 1H), 7.78-7.87 (m, 1H), 7.87-7.92 (m, 1H), 7.93-8.03 (m, 1H), 8.04-8.14 (m, 1H), 8.38-8.55 (m, 1H), 11.04-11.28 (m, 1H).

LCMS(ESI): [M]⁺ m/z: calcd 528.2; found 529.2; Rt=1.954 min.

### Example 113. The synthesis of 2-((2R,5S)-2-(2-(2-(dimethylamino)-2-methylpropyl)benzo[d]thiazol-5-yl)-5-methylpiperidin-1-yl)-2-oxo-N-(1H-pyrazolo[4,3-c]pyridin-7-yl)acetamide (Compound 122)

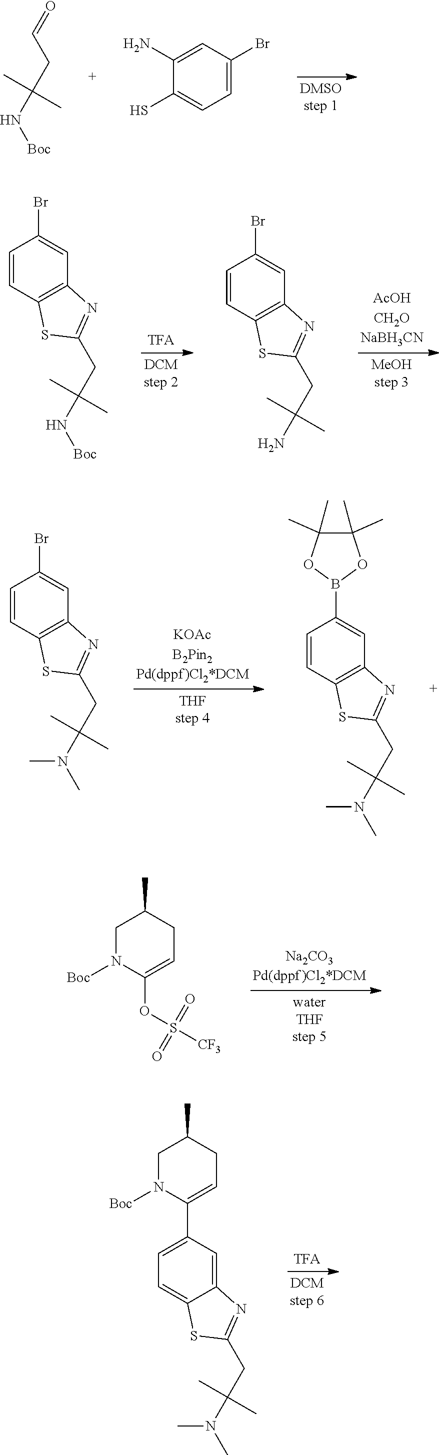

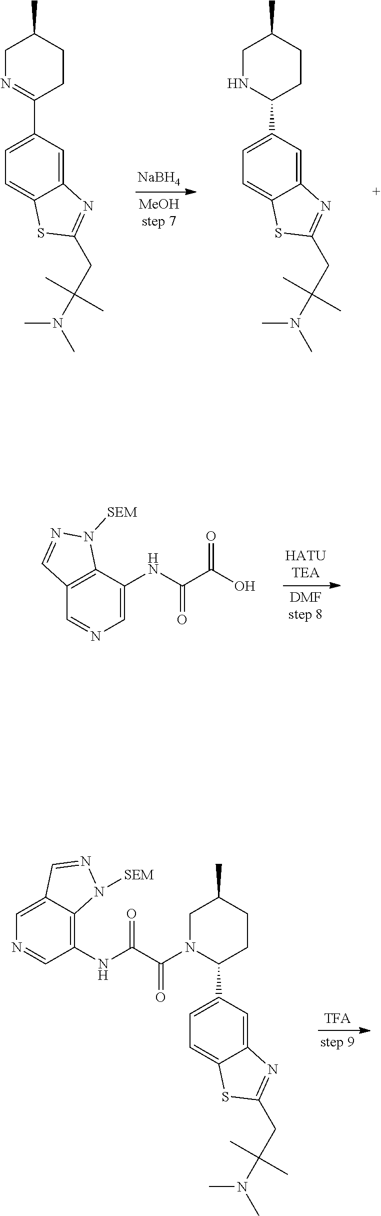 [SMILES: C[C@H]1CCC(c2ccc3sc(CC(C)(C)N(C)C)nc3c2)=NC1 ; "NaBH4" ; C[C@H]1CC[C@H](c2ccc3sc(CC(C)(C)N(C)C)nc3c2)NC1 ; C[Si](C)(C)CCOCn1ncc2cncc(NC(=O)C(=O)O)c21 ; C[C@H]1CC[C@H](c2ccc3sc(CC(C)(C)N(C)C)nc3c2)N(C(=O)C(=O)Nc2cncc3cnn(COCC[Si](C)(C)C)c23)C1]

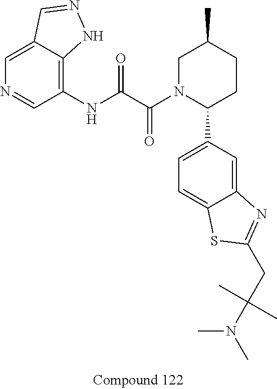 [SMILES: C[C@H]1CC[C@H](c2ccc3sc(CC(C)(C)N(C)C)nc3c2)N(C(=O)C(=O)Nc2cncc3cn[nH]c23)C1]

### Step 1: Synthesis of tert-butyl (1-(5-bromobenzo[d]thiazol-2-yl)-2-methylpropan-2-yl)carbamate

The synthesis of tert-butyl (1-(5-bromobenzo[d]thiazol-2-yl)-2-methylpropan-2-yl)carbamate is given by General Scheme S2, Step 1B. Yield: 6.14 g (76.66%). LCMS(ESI): [M-Boc]⁺ m/z: calcd 285.2; found 286.2; Rt=0.668 min.

### Step 2: Synthesis of 1-(5-bromobenzo[d]thiazol-2-yl)-2-methylpropan-2-amine

tert-Butyl N-[2-(5-bromo-1,3-benzothiazol-2-yl)-1,1-dimethyl-ethyl]carbamate (6.14 g, 15.93 mmol) was dissolved in DCM (20 mL) and TFA (20 mL) was added. The mixture was stirred for 1 hr at 25° C. Then it was evaporated, dissolved in water and Na₂CO₃ was added. The mixture was extracted with DCM twice, the combined organic phase was dried over Na₂SO₄ and concentrated under reduced pressure to obtain 1-(5-bromo-1,3-benzothiazol-2-yl)-2-methyl-propan-2-amine (4.5 g, 15.78 mmol, 99.02% yield). LCMS(ESI): [M]⁺ m/z: calcd 285.2; found 286.2; Rt=1.010 min.

### Step 3: Synthesis of 1-(5-bromobenzo[d]thiazol-2-yl)-N,N,2-trimethylpropan-2-amine

1-(5-Bromo-1,3-benzothiazol-2-yl)-2-methyl-propan-2-amine (4.5 g, 15.78 mmol) was dissolved in MeOH (40.46 mL) and formaldehyde, 37% in aq. soln., ACS, 36.5-38.0%, stab. with 10-15% MeOH (5.69 g, 189.34 mmol, 5.25 mL), sodium cyan borohydride (2.97 g, 47.33 mmol) and acetic acid (3.79 g, 63.11 mmol, 3.61 mL) was added. The reaction was stirred for 12 hr at 25° C. The reaction mixture was evaporated. Then H₂O was added and it was extracted with DCM twice, combined organic phase was dried over Na₂SO₄ and evaporated to obtain 1-(5-bromo-1,3-benzothiazol-2-yl)-N,N,2-trimethyl-propan-2-amine (4.2 g, 13.41 mmol, 84.98% yield). LCMS(ESI): [M]⁺ m/z: calcd 313.2; found 314.2; Rt=1.034 min.

### Step 4: Synthesis of N,N,2-trimethyl-1-(5-(4,4,5,5-tetramethyl-1,3,2-dioxaborolan-2-yl)benzo[d]thiazol-2-yl) propan-2-amine

The synthesis of N,N,2-trimethyl-1-(5-(4,4,5,5-tetramethyl-1,3,2-dioxaborolan-2-yl)benzo[d]thiazol-2-yl) propan-2-amine is given by General Scheme S2, Step 2. Yield: 4.37 g of crude. LCMS(ESI): [M]⁺ m/z: calcd 360.2; found 361.2; Rt=1.001 min.

### Step 5: Synthesis of(S)-tert-butyl 6-(2-(2-(dimethylamino)-2-methylpropyl)benzo[d]thiazol-5-yl)-3-methyl-3,4-dihydropyridine-1(2H)-carboxylate

The synthesis of(S)-tert-butyl 6-(2-(2-(dimethylamino)-2-methylpropyl)benzo[d]thiazol-5-yl)-3-methyl-3,4-dihydropyridine-1(2H)-carboxylate is given by General Scheme S2, Step 3. Yield: 7 g of crude. LCMS(ESI): [M]⁺ m/z: calcd 429.2; found 430.2; Rt=1.040 min.

### Step 6: Synthesis of(S)—N,N,2-trimethyl-1-(5-(5-methyl-3,4,5,6-tetrahydropyridin-2-yl)benzo[d]thiazol-2-yl) propan-2-amine

tert-Butyl (3S)-6-[2-[2-(dimethylamino)-2-methyl-propyl]-1,3-benzothiazol-5-yl]-3-methyl-3,4-dihydro-2H-pyridine-1-carboxylate (7 g, 16.29 mmol) was dissolved in DCM (20 mL) and TFA (20 mL) was added. The mixture was stirred for 1 hr at 25° C. Then it was evaporated, dissolved in water and filtered. To the filtrate Na₂CO₃ was added. The mixture was extracted with DCM twice, the combined organic phase was dried over Na₂SO₄ and concentrated under reduced pressure to obtain N,N,2-trimethyl-1-[5-[(3S)-3-methyl-2,3,4,5-tetrahydropyridin-6-yl]-1,3-benzothiazol-2-yl]propan-2-amine (2.05 g, crude). LCMS(ESI): [M]⁺ m/z: calcd 329.2; found 330.2; Rt=0.481 min.

### Step 7: Synthesis of N,N,2-trimethyl-1-(5-((2R,5S)-5-methylpiperidin-2-yl)benzo[d]thiazol-2-yl) propan-2-amine

The synthesis of N,N,2-trimethyl-1-(5-((2R,5S)-5-methylpiperidin-2-yl)benzo[d]thiazol-2-yl) propan-2-amine is given by General Scheme S2, Step 5. Yield: 1.14 g of crude. LCMS(ESI): [M]⁺ m/z: calcd 331.2; found 332.2; Rt=0.737 min.

### Step 8: Synthesis of 2-((2R,5S)-2-(2-(2-(dimethylamino)-2-methylpropyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

The synthesis of 2-((2R,5S)-2-(2-(2-(dimethylamino)-2-methylpropyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide is given by General Scheme S2, Step 6A. Yield: 25 mg (4.25%).

HPLC conditions: Column: XBridge BEH C18 100*19 mm, 5 microM; 0-1.5-5 min 60-60-65% water-MeCN+0.1% NH₄OH; (loading pump 4 ml/min MeCN). LCMS(ESI): [M]⁺ m/z: calcd 649.2; found 650.2; Rt=1.255 min.

### Step 9: Synthesis of 2-((2R,5S)-2-(2-(2-(dimethylamino)-2-methylpropyl)benzo[d]thiazol-5-yl)-5-methylpiperidin-1-yl)-2-oxo-N-(1H-pyrazolo[4,3-c]pyridin-7-yl)acetamide (Compound 122)

A solution of 2-oxo-2-[(2R,5S)-2-[2-[2-(dimethylamino)-2-methyl-propyl]-1,3-benzothiazol-5-yl]-5-methyl-1-piperidyl]-N-[1-(2-trimethylsilylethoxymethyl)pyrazolo[4,3-c]pyridin-7-yl]acetamide (25 mg, 38.47 μmol) in TFA (2.96 g, 25.96 mmol, 2 mL) was stirred at 25° C. for 3 hr. The solvent was evaporated and the residue was purified by HPLC (Device (Mobile Phase, Column): SYSTEM 40-70% 0-5 min H₂O/MeOH/0.1% NH₄OH, flow: 30 ml/min (loading pump 4 ml/min MeOH) target mass 520 column: XBridge BEH C18 5 um 130 A) to give 2-oxo-N-(1H-pyrazolo[4,3-c]pyridin-7-yl)-2-[(2R,5S)-2-[2-[2-(dimethylamino)-2-methyl-propyl]-1,3-benzothiazol-5-yl]-5-methyl-1-piperidyl]acetamide (6 mg, 11.55 μmol, 30.02% yield)

Compound 122: ¹H NMR (600 MHz, DMSO-d₆) δ (ppm) 1.00-1.15 (m, 9H), 1.33-1.46 (m, 1H), 1.71-1.81 (m, 1H), 1.85-1.99 (m, 1H), 2.12-2.33 (m, 7H), 2.87-3.22 (m, 4H), 3.77-4.13 (m, 1H), 5.58-5.77 (m, 1H), 7.32-7.45 (m, 1H), 7.85-7.92 (m, 1H), 7.96-8.06 (m, 1H), 8.29-8.39 (m, 1H), 8.42-8.55 (m, 1H), 8.88-8.99 (m, 1H), 11.05-11.20 (m, 1H), 12.99-13.11 (m, 1H). LCMS(ESI): [M]⁺ m/z: calcd 519.2; found 520.2; Rt=2.176 min.

### Example 114. Synthesis of 2-((2R,5S)-2-(2-(1,5-dimethyl-1,2,3,6-tetrahydropyridin-4-yl)benzo[d]thiazol-5-yl)-5-methylpiperidin-1-yl)-2-oxo-N-(1H-pyrazolo[4,3-b]pyridin-6-yl)acetamide (Compound 165)

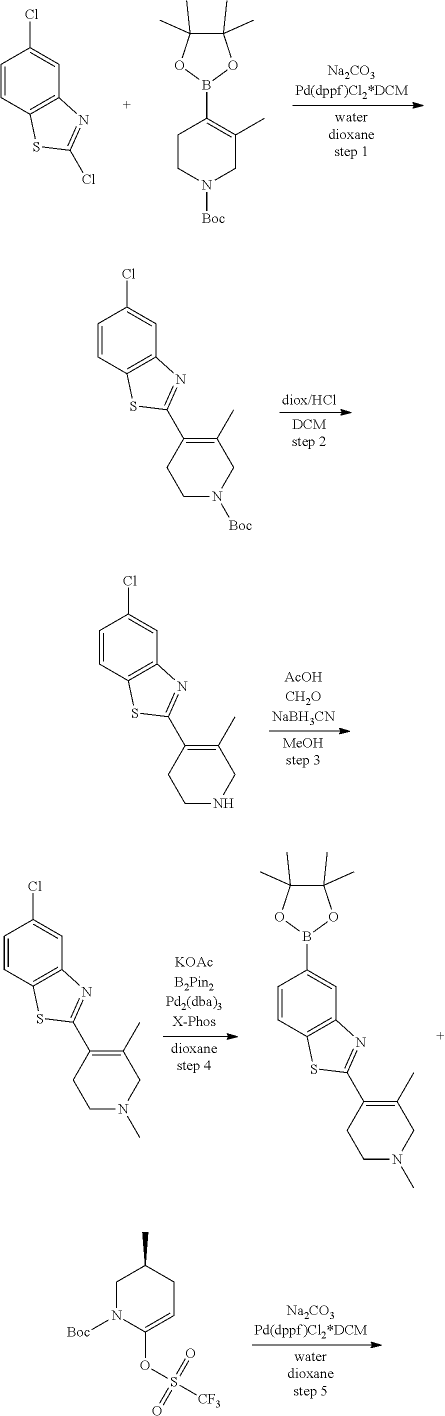

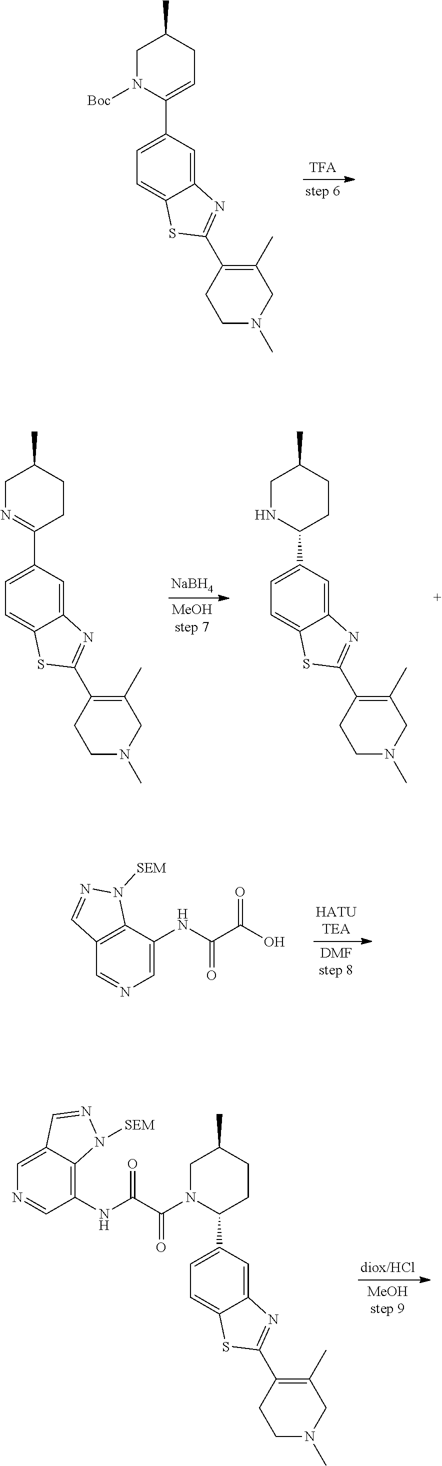 [SMILES: CC1=C(c2nc3cc(C4=CC[C@H](C)CN4C(=O)OC(C)(C)C)ccc3s2)CCN(C)C1 ; CC1=C(c2nc3cc(C4=NC[C@@H](C)CC4)ccc3s2)CCN(C)C1 ; "NaBH4" ; CC1=C(c2nc3cc([C@H]4CC[C@H](C)CN4)ccc3s2)CCN(C)C1 ; C[Si](C)(C)CCOCn1ncc2cncc(NC(=O)C(=O)O)c21 ; CC1=C(c2nc3cc([C@H]4CC[C@H](C)CN4C(=O)C(=O)Nc4cncc5cnn(COCC[Si](C)(C)C)c45)ccc3s2)CCN(C)C1]

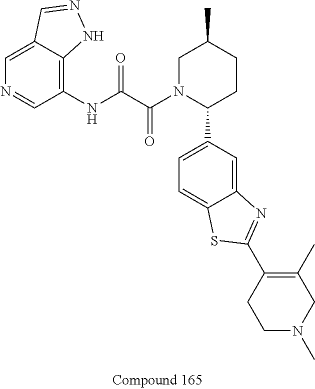 [SMILES: CC1=C(c2nc3cc([C@H]4CC[C@H](C)CN4C(=O)C(=O)Nc4cncc5cn[nH]c45)ccc3s2)CCN(C)C1]

### Step 1: Synthesis of tert-butyl 4-(5-chlorobenzo[d]thiazol-2-yl)-3-methyl-5,6-dihydropyridine-1(2H)-carboxylate

Sodium carbonate (19.48 g, 183.75 mmol, 7.69 mL) was added to a solution of 2,5-dichloro-1,3-benzothiazole (15 g, 73.50 mmol) and tert-butyl 5-methyl-4-(4,4,5,5-tetramethyl-1,3,2-dioxaborolan-2-yl)-3,6-dihydro-2H-pyridine-1-carboxylate (23.76 g, 73.50 mmol) in dioxane (200 mL) and water (65 mL). The reaction flask was evacuated and refilled with argon 3 times. Then, Pd(dppf)Cl₂*DCM (3.00 g, 3.68 mmol) was added under stream of argon. The resulting mixture was stirred at 95° C. for 15 hr under inert atmosphere. The reaction solution was decanted and concentrated under reduce pressure to give crude product which was diluted with MTBE (300 ml). The resulting cloudy solution was decanted from oily residue. MTBE was evaporated in vacuum to give tert-butyl 4-(5-chloro-1,3-benzothiazol-2-yl)-5-methyl-3,6-dihydro-2H-pyridine-1-carboxylate (18 g, 49.33 mmol, 67.11% yield). LCMS(ESI): [M]⁺ m/z: calcd 364.2; found 365.2; Rt=1.802 min.

### Step 2: Synthesis of 5-chloro-2-(5-methyl-1,2,3,6-tetrahydropyridin-4-yl)benzo[d]thiazole

To the stirred solution of tert-butyl 4-(5-chloro-1,3-benzothiazol-2-yl)-5-methyl-3,6-dihydro-2H-pyridine-1-carboxylate (18 g, 49.33 mmol) in DCM (150 mL) hydrogen chloride solution 4.0M in dioxane (80.00 g, 2.19 mol, 100 mL) was added. The resulting mixture was stirred at 25° C. for 14 hr. Solvents were evaporated in vacuum. The residue was diluted with MTBE (200 ml). The solid was filtered, washed with MTBE, then dried in vacuum to give 5-chloro-2-(5-methyl-1,2,3,6-tetrahydropyridin-4-yl)-1,3-benzothiazole (8.5 g, 28.22 mmol, 57.20% yield, HCl).

¹H NMR (500 MHZ, DMSO-d₆) δ (ppm) 2.12 (s, 3H), 2.85 (m, 2H), 3.26 (m, 2H), 3.75 (m, 2H), 7.49 (d, 1H), 8.08 (s, 1H), 8.16 (d, 1H), 9.73 (bds, 2H).

### Step 3: Synthesis of 5-chloro-2-(1,5-dimethyl-1,2,3,6-tetrahydropyridin-4-yl)benzo[d]thiazole

To the stirred solution of 5-chloro-2-(5-methyl-1,2,3,6-tetrahydropyridin-4-yl)-1,3-benzothiazole (8.5 g, 28.22 mmol, HCl) in MeOH (300 mL) formaldehyde, 37% w/w aq. soln., stab. with 7-8% MeOH (3.43 g, 42.33 mmol, 3.17 mL, 37% purity) and sodium acetate, anhydrous (5.79 g, 70.54 mmol, 3.79 mL) were added. The resulting mixture was stirred for 2 hr at 25° C. Then, sodium cyan borohydride (1.95 g, 31.04 mmol) was added portion wise. The resulting mixture was stirred at 25° C. for 12 hr. MeOH was evaporated. The residue was diluted with 10% aqueous sodium hydroxide solution (150 ml) and extracted with DCM (3*100 ml). The combined organic extracts were dried over sodium sulphate and concentrated in vacuum to afford crude 5-chloro-2-(1,5-dimethyl-3,6-dihydro-2H-pyridin-4-yl)-1,3-benzothiazole (6.1 g, 21.88 mmol, 77.54% yield). LCMS(ESI): [M]⁺ m/z: calcd 278.2; found 279.2; Rt=1.006 min.

### Step 4: Synthesis of 2-(1,5-dimethyl-1,2,3,6-tetrahydropyridin-4-yl)-5-(4,4,5,5-tetramethyl-1,3,2-dioxaborolan-2-yl)benzo[d]thiazole

5-Chloro-2-(1,5-dimethyl-3,6-dihydro-2H-pyridin-4-yl)-1,3-benzothiazole (6.2 g, 22.24 mmol), 4,4,5,5-tetramethyl-2-(4,4,5,5-tetramethyl-1,3,2-dioxaborolan-2-yl)-1,3,2-dioxaborolane (5.65 g, 22.24 mmol) and potassium acetate (4.37 g, 44.48 mmol, 2.78 mL) were mixed in dioxane (70 mL). The resulting mixture was evacuated and then backfilled with argon, this operation was repeated three times, then ris (dibenzylideneacetone)dipalladium (0) (1.02 g, 1.11 mmol) and XPhos (1.06 g, 2.22 mmol) were added under argon. The reaction mixture was stirred under argon at 90° C. for 15 hr. The reaction mixture was cooled down and filtered. The filter cake was washed with dioxane (2*10 ml) and discarded. The combined filtrate was concentrated in vacuum. The residue was diluted with MTBE (100 ml) and extracted with a NaHSO₄ water solution (30 ml) (repeated 3 times). The combined aqueous layer was basified to pH 10 with 10% aqueous sodium hydroxide to give water solution of 2-(1,5-dimethyl-3,6-dihydro-2H-pyridin-4-yl)-5-(4,4,5,5-tetramethyl-1,3,2-dioxaborolan-2-yl)-1,3-benzothiazole (8 g, 21.60 mmol, 97.14% yield) which was used directly in the next step.

LCMS(ESI): [M]⁺ m/z: calcd 370.2; found 371.2; Rt=1.178 min.

### Step 5: Synthesis of(S)-tert-butyl 6-(2-(1,5-dimethyl-1,2,3,6-tetrahydropyridin-4-yl)benzo[d]thiazol-5-yl)-3-methyl-3,4-dihydropyridine-1(2H)-carboxylate

Prepared by General Scheme S2, Step 3. Yield: 6 g of crude.

LCMS(ESI): [M]⁺ m/z: calcd 439.2; found 440.2; Rt=1.141 min.

### Step 6: Synthesis of(S)-2-(1,5-dimethyl-1,2,3,6-tetrahydropyridin-4-yl)-5-(5-methyl-3,4,5,6-tetrahydropyridin-2-yl)benzo[d]thiazole

tert-Butyl (3S)-6-[2-(1,5-dimethyl-3,6-dihydro-2H-pyridin-4-yl)-1,3-benzothiazol-5-yl]-3-methyl-3,4-dihydro-2H-pyridine-1-carboxylate (6 g, 13.65 mmol) was diluted with TFA (88.80 g, 778.79 mmol, 60.00 mL). The resulting mixture was stirred at 25° C. for 1.5 hr. TFA was evaporated in vacuum. The residue was diluted with water (150 ml). The resulting cloudy solution was decanted from oily residue, and then basified with NaHCO₃. The product was extracted with DCM (3*50 ml). Combined organic layers were dried over Na₂SO₄. DCM was evaporated in vacuum to give 2-(1,5-dimethyl-3,6-dihydro-2H-pyridin-4-yl)-5-[(3S)-3-methyl-2,3,4,5-tetrahydropyridin-6-yl]-1,3-benzothiazole (3.2 g, 9.43 mmol, 69.06% yield).

LCMS(ESI): [M]⁺ m/z: calcd 339.2; found 340.2; Rt=0.583 min.

### Step 7: Synthesis of 2-(1,5-dimethyl-1,2,3,6-tetrahydropyridin-4-yl)-5-((2R,5S)-5-methylpiperidin-2-yl)benzo[d]thiazole

The synthesis of 2-(1,5-dimethyl-1,2,3,6-tetrahydropyridin-4-yl)-5-((2R,5S)-5-methylpiperidin-2-yl)benzo[d]thiazole is given by General Scheme S2, Step 5. Yield: 2.3 g of crude. LCMS(ESI): [M]⁺ m/z: calcd 341.2; found 342.2; Rt=0.628 min.

### Step 8: Synthesis of 2-((2R,5S)-2-(2-(1,5-dimethyl-1,2,3,6-tetrahydropyridin-4-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

To the solution of 2-(1,5-dimethyl-3,6-dihydro-2H-pyridin-4-yl)-5-[(2R,5S)-5-methyl-2-piperidyl]-1,3-benzothiazole (169.19 mg, 495.42 μmol), 2-oxo-2-[[1-(2-trimethylsilylethoxymethyl)pyrazolo[4,3-b]pyridin-6-yl]amino]acetic acid (200 mg, 594.50 μmol) and TEA (150.39 mg, 1.49 mmol, 207.15 μL) in DMF (3 mL) HATU (207.21 mg, 544.96 μmol) was added portion wise. The mixture was stirred at 25° C. for 2 hr. The reaction mixture was poured into water (20 ml) and extracted with EtOAc (3*25 ml). Combined organic layers were washed with water (3*25 ml) dried over Na₂SO₄. EtOAc was evaporated in vacuum to give 2-oxo-2-[(2R,5S)-2-[2-(1,5-dimethyl-3,6-dihydro-2H-pyridin-4-yl)-1,3-benzothiazol-5-yl]-5-methyl-1-piperidyl]-N-[1-(2-trimethylsilylethoxymethyl)pyrazolo[4,3-b]pyridin-6-yl]acetamide (0.3 g, crude). LCMS(ESI): [M]⁺ m/z: calcd 659.2; found 660.2; Rt=0.958 min.

### Step 9: Synthesis of 2-((2R,5S)-2-(2-(1,5-dimethyl-1,2,3,6-tetrahydropyridin-4-yl)benzo[d]thiazol-5-yl)-5-methylpiperidin-1-yl)-2-oxo-N-(1H-pyrazolo[4,3-b]pyridin-6-yl)acetamide (Compound 165)

To the stirred solution of 2-oxo-2-[(2R,5S)-2-[2-(1,5-dimethyl-3,6-dihydro-2H-pyridin-4-yl)-1,3-benzothiazol-5-yl]-5-methyl-1-piperidyl]-N-[1-(2-trimethylsilylethoxymethyl)pyrazolo[4,3-b]pyridin-6-yl]acetamide (0.3 g, 454.60 μmol) in MeOH (5 mL) hydrogen chloride solution 4.0M in dioxane (800.00 mg, 21.94 mmol, 1 mL) was added. The resulting mixture was stirred at 25° C. for 15 hr. The reaction mixture was concentrated under reduce pressure and was then purified by reverse phase HPLC (SYSTEM 15-15-40% 0-5 min H₂O/MeCN/0.1% NH₄OH, flow: 30 ml/min (loading pump 4 ml/min MeCN) target mass 529.67 column: XBridge C18 100×19 mm, 5 um) to give 2-oxo-N-(1H-pyrazolo[4,3-b]pyridin-6-yl)-2-[(2R,5S)-2-[2-(1,5-dimethyl-3,6-dihydro-2H-pyridin-4-yl)-1,3-benzothiazol-5-yl]-5-methyl-1-piperidyl]acetamide (16 mg, 30.21 μmol, 6.64% yield).

Compound 165: ¹H NMR (600 MHz, DMSO-d₆) δ (ppm) 1.04-1.11 (m, 3H), 1.35-1.44 (m, 1H), 1.74-1.80 (m, 1H), 1.87-2.00 (m, 2H), 2.10-2.16 (m, 3H), 2.16-2.35 (m, 2H), 2.73-2.94 (m, 6H), 3.54-4.14 (m, 4H), 5.57-5.82 (m, 1H), 7.44-7.52 (m, 1H), 7.92-8.01 (m, 1H), 8.09-8.19 (m, 1H), 8.29-8.37 (m, 1H), 8.40-8.55 (m, 1H), 8.89-8.99 (m, 1H), 11.04-11.23 (m, 1H), 12.98-13.10 (m, 1H). LCMS(ESI): [M]⁺ m/z: calcd 529.2; found 530.2; Rt=2.235 min.

### Example 115. Synthesis of N-(3-amino-1H-pyrazolo[4,3-c]pyridin-7-yl)-2-oxo-2-[rac-(2R,5S)-2-(1,3-benzothiazol-5-yl)-5-methyl-1-piperidyl]acetamide (Compound 139)

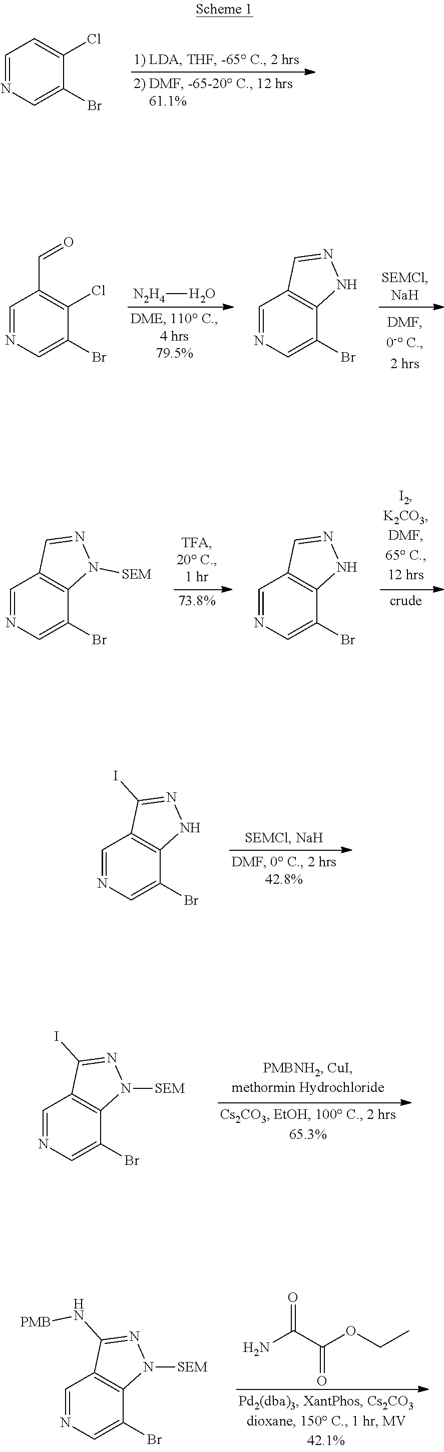

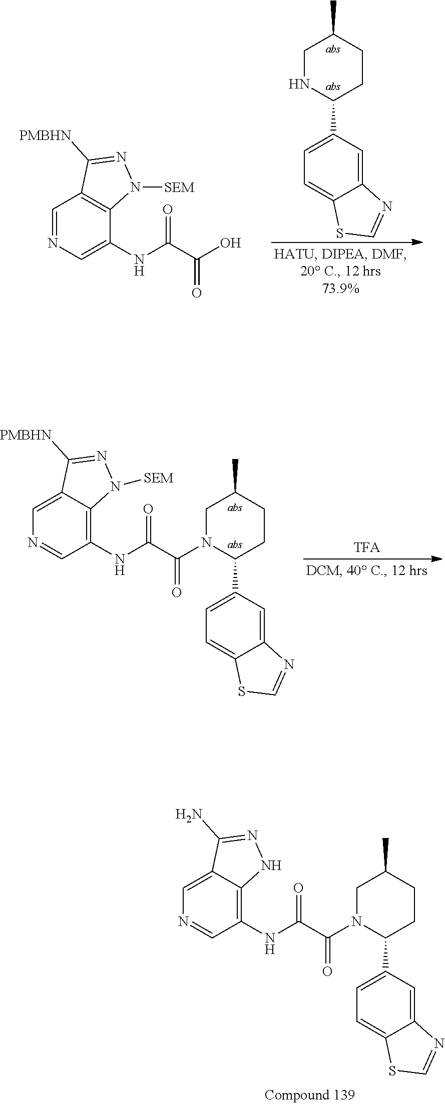

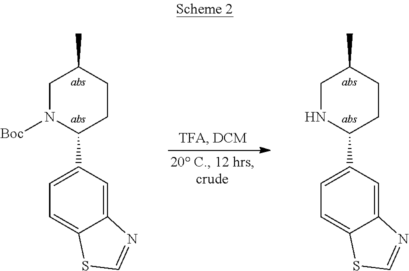 [SMILES: C[C@H]1CC[C@H](c2ccc3scnc3c2)N(C(=O)OC(C)(C)C)C1 ; C[C@H]1CC[C@H](c2ccc3scnc3c2)NC1]

### Step 1: Synthesis of 5-bromo-4-chloro-pyridine-3-carbaldehyde

To a mixture of LDA (2 M, 18 mL) in THF (50 mL) was added 3-bromo-4-chloro-pyridine (5 g, 25.9 mmol) in THF (50 mL) dropwise at −65° C. under nitrogen. The mixture was stirred at −65° C. for 2 hours. N,N-dimethylformamide (2.50 mL, 32.3 mmol) was added at −65° C. and the reaction was stirred at 20° C. for 12 hours. The resulting mixture was quenched by addition of NH₄Cl aqueous solution (100 mL) and extracted with EtOAc (100 mL*3). The combined organic layer was washed with saturated NH₄Cl aqueous solution (100 mL), brine (100 mL), dried over anhydrous Na₂SO₄, filtered and concentrated under reduced pressure to give a residue which was purified by flash chromatography (ISCO®; 40 g AgelaFlash® Silica Flash Column, petroleum ether/EtOAc with EtOAc from 0˜30%, flow rate=30 mL/min, 254 nm) to afford 5-bromo-4-chloro-pyridine-3-carbaldehyde (3.5 g, 61.1% yield) as yellow solid. LCMS(ESI) [M+H]⁺ m/z: calcd 221.9, found 221.9.

### Step 2: Synthesis of 7-bromo-1H-pyrazolo[4,3-c]pyridine

To a solution of 5-bromo-4-chloro-pyridine-3-carbaldehyde (3.5 g, 0.01589 mol) in DME (10 mL) was added hydrazine; hydrate (5.14 g, 87.2 mmol, 85% purity). The mixture was stirred at 110° C. for 4 hours. The resulting mixture was filtered and the filter cake was concentrated under reduced pressure to afford 7-bromo-1H-pyrazolo[4,3-c]pyridine (2.5 g, 79.5% yield) as a yellow solid. The crude product was used directly in next step without further purification.

### Step 3: Synthesis of 2-[(7-bromopyrazolo[4,3-c]pyridin-1-yl) methoxy]ethyl-trimethyl-silane

To a mixture of 7-bromo-1H-pyrazolo[4,3-c]pyridine (2 g, 10.10 mmol) in DMF (9.00 mL) was added NaH (760 mg, 19.0 mmol, 60% purity) slowly at 0° C. for 10 min, then 2-(chloromethoxy)ethyl-trimethyl-silane (2.00 mL, 11.3 mmol) was added dropwise. The resulting mixture was stirred at 0° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®, 25 g AgelaFlash® Silica Flash Column, petroleum ether/EtOAc with EtOAc from 0˜50%, flow rate=30 mL/min, 254 nm) to afford 2-[(7-bromopyrazolo[4,3-c]pyridin-1-yl) methoxy]ethyl-trimethyl-silane (1.5 g) as brown oil and 2-[(7-bromopyrazolo[4,3-c]pyridin-2-yl) methoxy]ethyl-trimethyl-silane (0.5 g) as brown oil. ¹H NMR (400 MHZ, methanol-d₄) δ ppm 9.04 (s, 1H), 8.52 (s, 1H), 8.36 (s, 1H), 6.05 (s, 2H), 3.63 (t, J=7.9 Hz, 2H), 0.84 (t, J=7.8 Hz, 2H), −0.10 (s, 9H); LCMS (ESI) [M+H]⁺ m/z: calcd 328.0, found 328.0.

### Step 4: Synthesis of 7-bromo-1H-pyrazolo[4,3-c]pyridine

A mixture of 2-[(7-bromopyrazolo[4,3-c]pyridin-1-yl) methoxy]ethyl-trimethyl-silane (1 g, 3.05 mmol) and TFA (4 mL) was stirred at 20° C. for 1 hour. The resulting mixture was concentrated under reduced pressure to afford 7-bromo-1H-pyrazolo[4,3-c]pyridine (700 mg, 73.9% yield, TFA) as a yellow oil.

### Step 5: Synthesis of 7-bromo-3-iodo-1H-pyrazolo[4,3-c]pyridine

To a solution of 7-bromo-1H-pyrazolo[4,3-c]pyridine (700 mg, 3.53 mmol), K₂CO₃ (1.47 g, 10.6 mmol) in DMF (4 mL) was added I₂ (1.40 g, 5.52 mmol). The mixture was stirred at 65° C. for 12 hours. The resulting mixture was quenched by addition of water (30 mL) and extracted with EtOAc (50 mL*3). The combined organic layer was washed with brine (30 mL), dried over anhydrous Na₂SO₄, filtered and concentrated under reduced pressure to afford 7-bromo-3-iodo-1H-pyrazolo[4,3-c]pyridine (1 g, crude) as a yellow oil.

### Step 6: Synthesis of 2-[(7-bromo-3-iodo-pyrazolo[4,3-c]pyridin-1-yl) methoxy]ethyl-trimethyl-silane

To a solution of 7-bromo-3-iodo-1H-pyrazolo[4,3-c]pyridine (1 g, 3.09 mmol) in DMF (10 mL) was added NaH (200 mg, 5.00 mmol, 60 wt % in mineral oil) at 0° C. The mixture was stirred at 0° C. for 2 hours. The resulting mixture was quenched by addition of water (50 mL) and extracted with EtOAc (50 mL*3). The combined organic layer was washed with saturated NH₄Cl aqueous solution (50 mL*2), brine (50 mL), dried over anhydrous Na₂SO₄, filtered and concentrated under reduced pressure to give a residue which was purified by flash chromatography (ISCO®; 20 g AgelaFlash® Silica Flash Column, petroleum ether/EtOAc with EtOAc from 0˜30%, flow rate=30 mL/min, 254 nm) to afford 2-[(7-bromo-3-iodo-pyrazolo[4,3-c]pyridin-1-yl) methoxy]ethyl-trimethyl-silane (600 mg, 42.8% yield) as yellow solid. ¹H NMR (400 MHZ, methanol-d₄) δ ppm 8.74 (s, 1H), 8.61 (s, 1H), 6.03 (s, 2H), 3.65 (t, J=7.9 Hz, 2H), 0.84 (t, J=7.9 Hz, 2H), −0.14-−0.05 (m, 9H).

LCMS(ESI) [M+H]⁺ m/z: calcd 455.9, found 455.9.

### Step 7: Synthesis of 7-bromo-N-[(4-methoxyphenyl)methyl]-1-(2-trimethylsilylethoxymethyl)pyrazolo[4,3-c]pyridin-3-amine

To a solution of 2-[(7-bromo-3-iodo-pyrazolo[4,3-c]pyridin-1-yl) methoxy]ethyl-trimethyl-silane (600 mg, 1.32 mmol), (4-methoxyphenyl) methanamine (240 mg, 1.75 mmol), Cs₂CO₃ (900 mg, 2.76 mmol), 3-carbamimidoyl-1,1-dimethyl-guanidine; hydrochloride (90 mg, 0.543 mmol) in EtOH (10 mL) was added CuI (60 mg, 0.315 mmol). The resulting mixture was sealed and degassed under vacuum and purged with N₂ for three times, and then stirred at 100° C. for 2 hours under N₂. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 12 g AgelaFlash® Silica Flash Column, petroleum ether/EtOAc with EtOAc from 0˜30%, flow rate=30 mL/min, 254 nm) to afford 7-bromo-N-[(4-methoxyphenyl)methyl]-1-(2-trimethylsilylethoxymethyl)pyrazolo[4,3-c]pyridin-3-amine (400 mg, 65.3% yield) as yellow oil. LCMS(ESI) [M+H]⁺ m/z: calcd 465.1, found 465.0.

### Step 8: Synthesis of 2-[[3-[(4-methoxyphenyl)methylamino]-1-(2-trimethylsilylethoxymethyl)pyrazolo[4,3-c]pyridin-7-yl]amino]-2-oxo-acetic acid

To a solution of 7-bromo-N-[(4-methoxyphenyl)methyl]-1-(2-trimethylsilylethoxymethyl)pyrazolo[4,3-c]pyridin-3-amine (350 mg, 0.755 mmol), ethyl 2-amino-2-oxo-acetate (170 mg, 1.45 mmol), XantPhos (105 mg, 0.181 mmol), Pd₂(dba)₃ (70 mg, 0.076 mmol) in dioxane (10 mL) was added Cs₂CO₃ (700 mg, 2.15 mmol). The resulting mixture was degassed under vacuum and purged with nitrogen three times and sealed, then stirred at 150° C. for 1 hour under microwave. The resulting mixture was quenched by addition of water (30 mL) and extracted with EtOAc (30 mL*3). The aqueous solution was adjusted to pH=4 with 2M HCl aqueous solution and extracted with DCM (40 mL*3) dried over anhydrous Na₂SO₄, filtered and concentrated under reduced pressure. The residue was purified by flash chromatography (Biotage®, Column: SepaFlash® Sphercial C18, 40 g, 40-60 μm, 120 Å; MeCN/water (0.05% NH₃—H₂O) with MeCN from 0˜35%, 30 mL/min, 254 nm) to afford 2-[[3-[(4-methoxyphenyl)methylamino]-1-(2-trimethylsilylethoxymethyl)pyrazolo[4,3-c]pyridin-7-yl]amino]-2-oxo-acetic acid (150 mg, 42.1% yield) as a yellow solid. LCMS(ESI) [M+H]⁺ m/z: calcd 472.2, found 472.2.

### Step 9: Synthesis of 5-[rac-(2R,5S)-5-methyl-2-piperidyl]-1,3

To a solution of tert-butyl rac-(2R,5S)-2-(1,3-benzothiazol-5-yl)-5-methyl-piperidine-1-carboxylate (100 mg, 301 μmol) in DCM (4 mL) was added TFA (500 μL, 6.49 mmol). The mixture was stirred at 20° C. for 12 hours. The resulting mixture was concentrated under reduced pressure to afford 5-[rac-(2R,5S)-5-methyl-2-piperidyl]-1,3-benzothiazole (80 mg, crude, TFA) as colorless oil. LCMS(ESI) [M+H]⁺ m/z: calcd 233.1, found 233.0.

### Step 10: Synthesis of [rac-(2R,5S)-2-(1,3-benzothiazol-5-yl)-5-methyl-1-piperidyl] 2-[[3-[(4-methoxyphenyl)methylamino]-1-(2-trimethylsilylethoxymethyl)pyrazolo[4,3-c]pyridin-7-yl]amino]-2-oxo-acetate

To a solution of 2-[[3-[(4-methoxyphenyl)methylamino]-1-(2-trimethylsilylethoxymethyl)pyrazolo[4,3-c]pyridin-7-yl]amino]-2-oxo-acetic acid (100 mg, 0.212 mol), 5-[rac-(2R,5S)-5-methyl-2-piperidyl]-1,3-benzothiazole (75 mg, 0.217 mmol, TFA), HATU (150 mg, 0.395 mmol) in DMF (4 mL) was added N-ethyl-N-isopropyl-propan-2-amine (300 μL, 1.72 mmol). The mixture was stirred at 20° C. for 12 hours. The resulting mixture was quenched by addition of water (30 mL) and extracted with EtOAc (30 mL*3). The combined organic layer was washed with saturated NH₄Cl aqueous solution (30 mL*2), brine (30 mL), dried over anhydrous Na₂SO₄, filtered and concentrated under reduced pressure. The residue was purified by flash chromatography (ISCO®; 12 g AgelaFlash® Silica Flash Column, petroleum ether/EtOAc with EtOAc from 0˜80%, flow rate=30 mL/min, 254 nm) to afford [rac-(2R,5S)-2-(1,3-benzothiazol-5-yl)-5-methyl-1-piperidyl] 2-[[3-[(4-methoxyphenyl)methylamino]-1-(2-trimethylsilylethoxymethyl)pyrazolo[4,3-c]pyridin-7-yl]amino]-2-oxo-acetate (110 mg, 73.9% yield) as yellow oil; LCMS(ESI) [M+H]⁺ m/z: calcd 686.3, found 686.3.

### Step 11: Synthesis of N-(3-amino-1H-pyrazolo[4,3-c]pyridin-7-yl)-2-oxo-2-[rac-(2R,5S)-2-(1,3-benzothiazol-5-yl)-5-methyl-1-piperidyl]acetamide ((Compound 139)

To a solution of N-[3-[(4-methoxyphenyl)methylamino]-1-(2-trimethylsilylethoxymethyl)pyrazolo[4,3-c]pyridin-7-yl]-2-oxo-2-[rac-(2R,5S)-2-(1,3-benzothiazol-5-yl)-5-methyl-1-piperidyl]acetamide (120 mg, 0.175 mmol) in DCM (2 mL) was added TFA (2 mL, 0.0259 mol). The mixture was stirred at 40° C. for 12 hours. The resulting mixture was concentrated under reduced pressure to give a residue which was purified by preparative HPLC (Instrument: Gilson GX-281 Liquid Handler, Gilson 322 Pump, Gilson 156 UV Detector; Column: Phenomenex Gemini C18 75×40 mm×3 μm; Mobile phase A: H₂O with NH₄HCO₃ (v %); Mobile phase B: MeCN; Gradient: B from 28% to 58% in 9.5 min, hold 100% B for 2 min; Flow Rate: 30 mL/min; Column Temperature: 30° C.; Wavelength: 220 nm, 254 nm) to give a crude product which was purified by preparative HPLC (Instrument: Gilson GX-281 Liquid Handler, Gilson 322 Pump, Gilson 156 UV Detector; Column: ACE5 C18 AR Gemini C18 150×30 mm×5 um; Mobile phase A: H₂O with FA (v %); Mobile phase B: MeCN; Gradient: B from 20% to 50% in 8.5 min, hold 100% B for 2 min; Flow Rate: 30 mL/min; Column Temperature: 30° C.; Wavelength: 220 nm, 254 nm) to afford N-(3-amino-1H-pyrazolo[4,3-c]pyridin-7-yl)-2-oxo-2-[rac-(2R,5S)-2-(1,3-benzothiazol-5-yl)-5-methyl-1-piperidyl]acetamide (10 mg, 13.1% yield) as yellow solid.

¹H NMR (400 MHZ, methanol-d₄) δ ppm 9.19-9.35 (m, 1H), 8.74-8.94 (m, 1H), 8.32-8.59 (m, 1H), 8.04-8.26 (m, 2H), 7.55 (br d, J=8.5 Hz, 1H), 5.58-6.04 (m, 1H), 3.87-4.23 (m, 1H), 3.38-3.58 (m, 1H), 2.38 (br s, 2H), 1.83-2.12 (m, 2H), 1.28-1.55 (m, 1H), 1.17 (d, J=7.0 Hz, ³H); LCMS(ESI) [M+H]⁺ m/z: calcd 436.1, found 436.1; HPLC: 99.52%@254 nm; SFC: 97.56% ee.

### Example 116. Synthesis of N-(4-amino-1H-pyrazolo[4,3-c]pyridin-7-yl)-2-(rac-(2S,5R)-5-methyl-2-(1-methyl-1H-pyrazol-5-yl)piperidin-1-yl)-2-oxoacetamide (Compound 115)

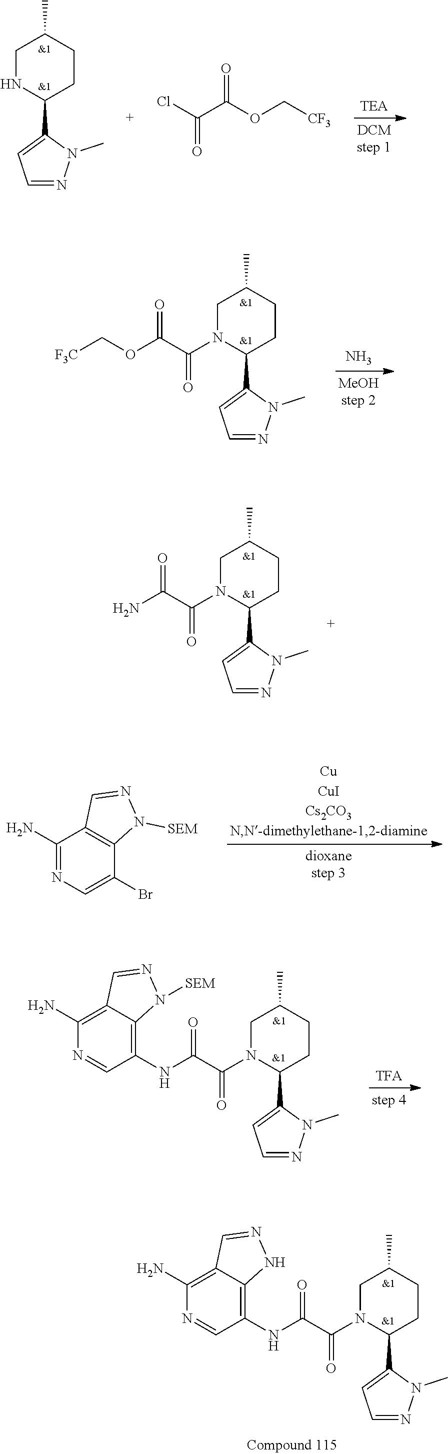

The synthesis of 5-methyl-2-(1-methyl-1H-pyrazol-5-yl)piperidine is given by the following procedure. Sodium borohydride (725.67 mg, 19.18 mmol, 678.20 μL) was added in one portion to a stirred solution of 3-methyl-6-(2-methylpyrazol-3-yl)-2,3,4,5-tetrahydropyridine (1.7 g, 9.59 mmol) in methanol (20 mL) at 0° C. The resulting mixture was stirred at 0° C. for 1 hr, and then evaporated in vacuo. The residue was diluted with water (20 ml) and extracted with dichloromethane (2*40 ml). The combined organic extracts were dried over sodium sulphate and evaporated in vacuo to afford 5-methyl-2-(2-methylpyrazol-3-yl)piperidine (1.3 g, 7.25 mmol, 75.61% yield) as yellow oil, which was used directly in the next step.

¹H NMR (400 MHZ, CDCl₃) δ (ppm) 0.89 (d, 3H), 1.15 (m, 2H), 1.58 (m, 2H), 1.88 (m, 2H), 2.37 (m, 1H), 3.11 (m, 1H), 3.64 (m, 1H), 3.88 (s, 3H), 6.14 (d, 1H), 7.37 (d, 1H).

LCMS(ESI): [M+1] m/z: calcd 179.2; found 180.2; Rt=0.749 min.

### Step 1: Synthesis of 2,2,2-trifluoroethyl 2-(rac-(2S,5R)-5-methyl-2-(1-methyl-1H-pyrazol-5-yl)piperidin-1-yl)-2-oxoacetate

Prepared by general procedure scheme S9 step 1. Yield: 0.43 g (66.08%).

LCMS(ESI): [M]⁺ m/z: calcd 333.2; found 334.2; Rt=1.301 min.

### Step 2: Synthesis of 2-(rac-(2S,5R)-5-methyl-2-(1-methyl-1H-pyrazol-5-yl)piperidin-1-yl)-2-oxoacetamide

2,2,2-Trifluoroethyl 2-oxo-2-[rac-(2S,5R)-5-methyl-2-(2-methylpyrazol-3-yl)-1-piperidyl]acetate (0.43 g, 1.29 mmol) was dissolved in MeOH/NH₃ (20 mL) as stirred overnight at 20° C. The reaction mixture was evaporated to dryness. The obtained product was used in the next step without further purification. 2-Oxo-2-[rac-(2S,5R)-5-methyl-2-(2-methylpyrazol-3-yl)-1-piperidyl]acetamide (0.3 g, 1.20 mmol, 92.91% yield) was obtained as a white solid.

LCMS(ESI): [M]⁺ m/z: calcd 250.2; found 251.2; Rt=0.670 min.

### Step 3: Synthesis of N-(4-amino-1-((2-(trimethylsilyl)ethoxy)methyl)-1H-pyrazolo[4,3-c]pyridin-7-yl)-2-(rac-(2S,5R)-5-methyl-2-(1-methyl-1H-pyrazol-5-yl)piperidin-1-yl)-2-oxoacetamide

Prepared by general procedure scheme S9 step 3A. Yield: 110 mg (17.90%).

HPLC conditions: Column: SunFire C18 100*19 mm, 5 microM; 2-10 min 10-50% MeCN+FA 30 ml/min; (loading pump 4 ml/min MeCN+FA).

LCMS(ESI): [M]⁺ m/z: calcd 512.2; found 513.2; Rt=1.277 min.

### Step 4: Synthesis of N-(4-amino-1H-pyrazolo[4,3-c]pyridin-7-yl)-2-(rac-(2S,5R)-5-methyl-2-(1-methyl-1H-pyrazol-5-yl)piperidin-1-yl)-2-oxoacetamide (Compound 115)

N-[4-Amino-1-(2-trimethylsilylethoxymethyl)pyrazolo[4,3-c]pyridin-7-yl]-2-oxo-2-[rac-(2S,5R)-5-methyl-2-(2-methylpyrazol-3-yl)-1-piperidyl]acetamide (0.11 g, 214.56 μmol) was dissolved in TFA (733.94 mg, 6.44 mmol, 495.91 μL) and stirred at 20° C. for 3 hr. The reaction mixture was submitted to HPLC (2-10 min 30-65% MeOH+NH₃ 30/min; loading pump 4 ml/min MeOH+NH₃ column SunFire 19*100 mm). N-(4-Amino-1H-pyrazolo[4,3-c]pyridin-7-yl)-2-oxo-2-[rac-(2S,5R)-5-methyl-2-(2-methyl pyrazol-3-yl)-1-piperidyl]acetamide (0.0316 g, 82.63 μmol, 38.51% yield) was obtained as a light-yellow solid.

### Compound 115:

¹H NMR (600 MHz, DMSO-d₆) δ (ppm) 0.78 (m, 3H), 1.61 (m, 3H), 1.91 (m, 1H), 2.09 (m, 1H), 3.15 (m, 1H), 3.76 (m, 4H), 5.69 (m, 1H), 6.34 (m, 1H), 6.75 (m, 2H), 7.36 (m, 1H), 7.64 (m, 1H), 8.15 (m, 1H), 10.47 (m, 1H), 12.75 (m, 1H).

LCMS(ESI): [M]⁺ m/z: calcd 382.2; found 383.2; Rt=1.634 min.

### Example 117. Synthesis of N-(4-amino-1H-pyrazolo[4,3-c]pyridin-7-yl)-2-(2-(4-fluorophenyl)-4-methoxy-5-methylpiperidin-1-yl)-2-oxoacetamide (Compound 38, Compound 7, Compound 160 and Compound 116)

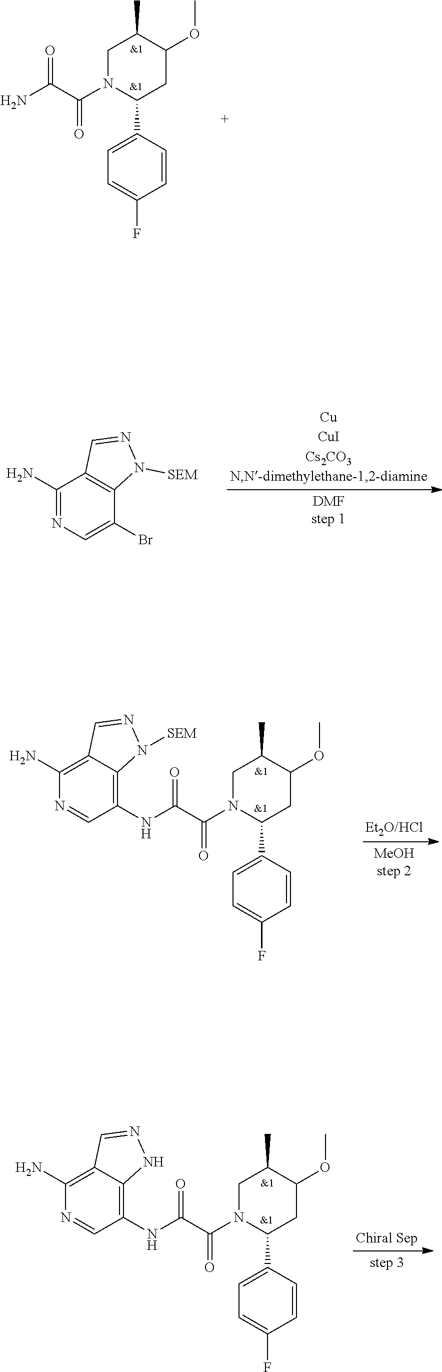 [SMILES: COC1C[C@H](c2ccc(F)cc2)N(C(=O)C(N)=O)C[C@H]1C ; C[Si](C)(C)CCOCn1ncc2c(N)ncc(Br)c21 ; COC1C[C@H](c2ccc(F)cc2)N(C(=O)C(=O)Nc2cnc(N)c3cnn(COCC[Si](C)(C)C)c23)C[C@H]1C ; COC1C[C@H](c2ccc(F)cc2)N(C(=O)C(=O)Nc2cnc(N)c3cn[nH]c23)C[C@H]1C]

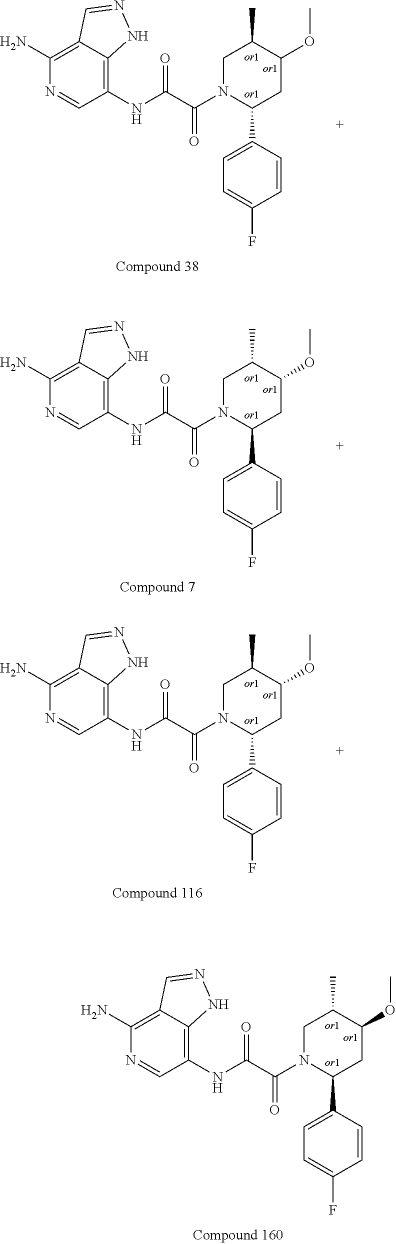 [SMILES: COC1C[C@H](c2ccc(F)cc2)N(C(=O)C(=O)Nc2cnc(N)c3cn[nH]c23)C[C@H]1C ; CO[C@@H]1C[C@@H](c2ccc(F)cc2)N(C(=O)C(=O)Nc2cnc(N)c3cn[nH]c23)C[C@@H]1C ; CO[C@@H]1C[C@H](c2ccc(F)cc2)N(C(=O)C(=O)Nc2cnc(N)c3cn[nH]c23)C[C@H]1C ; CO[C@H]1C[C@@H](c2ccc(F)cc2)N(C(=O)C(=O)Nc2cnc(N)c3cn[nH]c23)C[C@@H]1C]

The synthesis of 2-oxo-2-[rac-(2R,5R)-2-(4-fluorophenyl)-4-methoxy-5-methyl-1-piperidyl]acetamide is given by Intermediate 47.

### Step 1: Synthesis of N-(4-amino-1-((2-(trimethylsilyl)ethoxy)methyl)-1H-pyrazolo[4,3-c]pyridin-7-yl)-2-(2-(4-fluorophenyl)-4-methoxy-5-methylpiperidin-1-yl)-2-oxoacetamide

Prepared by general procedure scheme S9 step 3A. Yield: 322 mg (29.22%).

HPLC conditions: Column: SunFire C18 100*19 mm, 5 microM; 2-10 min 30-60% MeCN+FA 30 ml/min; (loading pump 4 ml/min MeCN).

LCMS(ESI): [M]⁺ m/z: calcd 556.2; found 557.2; Rt=1.130 min.

### Step 2: Synthesis of N-(4-amino-1H-pyrazolo[4,3-c]pyridin-7-yl)-2-(2-(4-fluorophenyl)-4-methoxy-5-methylpiperidin-1-yl)-2-oxoacetamide

N-[4-Amino-1-(2-trimethylsilylethoxymethyl)pyrazolo[4,3-c]pyridin-7-yl]-2-oxo-2-[rac-(2R,5R)-2-(4-fluorophenyl)-4-methoxy-5-methyl-1-piperidyl]acetamide (0.322 g, 496.34 μmol, 2HCOOH) was dissolved in MeOH (4 mL) then Et₂O (HCl) (4 mL) was added the resulting mixture and stirred at 20° C. for 12 hr. The reaction mixture was submitted to HPLC (2-10 min 30-60% MeCN+NH₃ 30/min; loading pump 4 ml/min MeCN+NH₃ column SunFire 19*100 mm). N-(4-Amino-1H-pyrazolo[4,3-c]pyridin-7-yl)-2-oxo-2-[rac-(2R,5R)-2-(4-fluorophenyl)-4-methoxy-5-methyl-1-piperidyl]acetamide (0.117 g, 274.36 μmol, 55.28% yield) was obtained as a light-yellow solid.

LCMS(ESI): [M]⁺ m/z: calcd 426.2; found 427.2; Rt=0.781 min.

### Step 3: Chiral Separation (Compound 38, Compound 7, Compound 160 and Compound 116)

Racemic N-(4-amino-1H-pyrazolo[4,3-c]pyridin-7-yl)-2-(2-(4-fluorophenyl)-4-methoxy-5-methylpiperidin-1-yl)-2-oxoacetamide (117 mg, 274.36 μmol) was chiral separated (Chiralcel OJ-H (250*30, 5 mkm), Hexane-IPA-MeOH, 75-15-15, 30 ml/min) to obtain N-(4-amino-1H-pyrazolo[4,3-c]pyridin-7-yl)-2-oxo-2-[(2R,4S,5R)-2-(4-fluorophenyl)-4-methoxy-5-methyl-1-piperidyl]acetamide (0.022 g, 51.59 μmol, 18.80% yield) (RT=23.74 min), N-(4-amino-1H-pyrazolo[4,3-c]pyridin-7-yl)-2-oxo-2-[(2S,4R,5S)-2-(4-fluorophenyl)-4-methoxy-5-methyl-1-piperidyl]acetamide (0.028 g, 65.66 μmol, 23.93% yield) (RT=43.22 min), N-(4-amino-1H-pyrazolo[4,3-c]pyridin-7-yl)-2-oxo-2-[(2R,4R, 5R)-2-(4-fluorophenyl)-4-methoxy-5-methyl-1-piperidyl]acetamide (RT=29.31 min) and N-(4-amino-1H-pyrazolo[4,3-c]pyridin-7-yl)-2-oxo-2-[(2S,4S,5S)-2-(4-fluorophenyl)-4-methoxy-5-methyl-1-piperidyl]acetamide (RT=80.94 min).

Rel Time for Compound 7 in analytical conditions (column: OD-H, Hexane-IPA-MeOH, 50-25-25, 0.6 ml/min as mobile phase) 11.30 min, for Compound 38 19.61 min, for Compound 116 14.39 min and for Compound 160 50.36 min.

Compound 38: Retention time: 19.61 min.

LCMS(ESI): [M]⁺ m/z: calcd 426.2; found 427.2; Rt=1.723 min.

Compound 7: Retention time: 11.30 min.

¹H NMR (600 MHZ, DMSO-d₆) δ (ppm) 0.93-1.05 (m, 3H), 1.84-1.97 (m, 1H), 2.19-2.26 (m, 1H), 2.89-3.09 (m, 3H), 3.12-3.20 (m, 1H), 3.57-3.69 (m, 1H), 3.70-3.92 (m, 1H), 3.95-5.15 (m, 1H), 5.34-6.31 (m, 1H), 6.60-6.83 (m, 2H), 6.95-7.11 (m, 1H), 7.13-7.19 (m, 1H), 7.31-7.38 (m, 2H), 7.45-7.77 (m, 1H), 8.07-8.27 (m, 1H), 9.46-10.56 (m, 1H), 12.51-13.44 (m, 1H).

LCMS(ESI): [M]⁺ m/z: calcd 426.2; found 427.2; Rt=1.718 min.

Compound 116: Retention time: 14.39 min.

¹H NMR (600 MHz, DMSO-d₆) δ (ppm) 0.78-0.98 (m, 3H), 1.77-2.08 (m, 1H), 2.09-2.37 (m, 1H), 3.00-3.23 (m, 4H), 3.59-3.70 (m, 1H), 3.82-4.00 (m, 1H), 4.23-5.16 (m, 1H), 5.36-6.34 (m, 1H), 6.66-7.06 (m, 2H), 7.15-7.33 (m, 2H), 7.35-7.46 (m, 2H), 7.46-7.78 (m, 1H), 8.04-8.29 (m, 1H), 9.54-10.72 (m, 1H), 12.51-13.48 (m, 1H).

LCMS(ESI): [M]⁺ m/z: calcd 426.2; found 427.2; Rt=1.787 min.

Compound 160: Retention time: 50.36 min.

¹H NMR (600 MHz, DMSO-d₆) δ (ppm) 0.78-1.04 (m, 3H), 1.79-2.08 (m, 1H), 2.10-2.36 (m, 1H), 2.90-3.22 (m, 4H), 3.42-3.66 (m, 1H), 3.71-4.01 (m, 1H), 4.19-5.16 (m, 1H), 5.31-6.38 (m, 1H), 6.60-6.91 (m, 2H), 7.02-7.28 (m, 2H), 7.27-7.45 (m, 2H), 7.46-7.76 (m, 1H), 8.07-8.27 (m, 1H), 9.43-10.65 (m, 1H), 12.32-13.70 (m, 1H).

LCMS(ESI): [M]⁺ m/z: calcd 426.2; found 427.2; Rt=1.785 min.

### Example 118. Synthesis of 2-((2R,5S)-5-methyl-2-(2-((S)-1-methylpyrrolidin-3-yl)-2H-indazol-6-yl)piperidin-1-yl)-2-oxo-N-(1H-pyrazolo[4,3-c]pyridin-7-yl)acetamide Compound 117

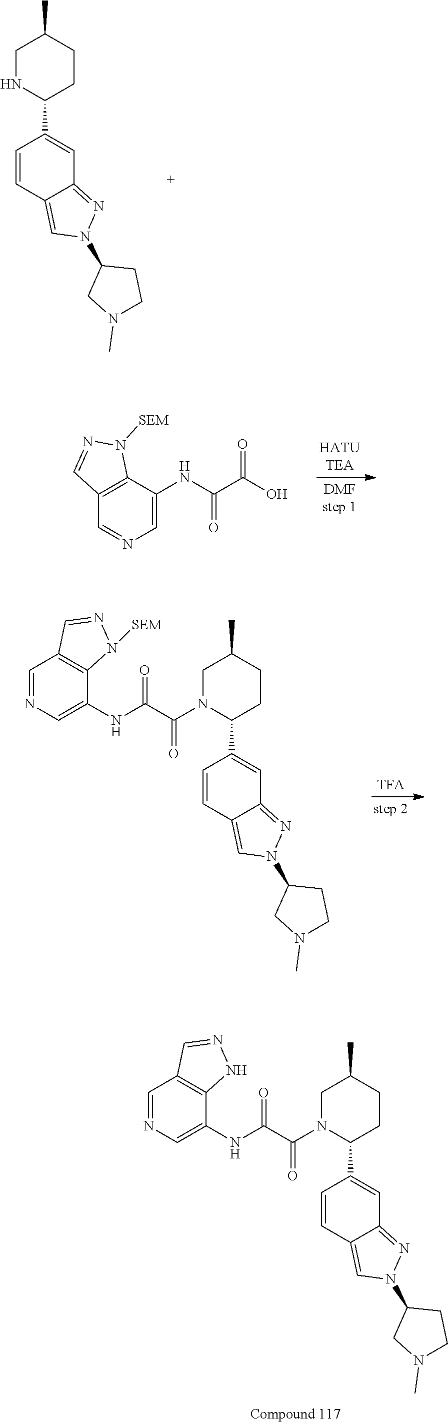 [SMILES: C[C@H]1CC[C@H](c2ccc3cn([C@H]4CCN(C)C4)nc3c2)NC1 ; C[Si](C)(C)CCOCn1ncc2cncc(NC(=O)C(=O)O)c21 ; C[C@H]1CC[C@H](c2ccc3cn([C@H]4CCN(C)C4)nc3c2)N(C(=O)C(=O)Nc2cncc3cnn(COCC[Si](C)(C)C)c23)C1 ; C[C@H]1CC[C@H](c2ccc3cn([C@H]4CCN(C)C4)nc3c2)N(C(=O)C(=O)Nc2cncc3cn[nH]c23)C1]

The synthesis of 6-((2R,5S)-5-methylpiperidin-2-yl)-2-((S)-1-methylpyrrolidin-3-yl)-2H-indazole is prepared by general procedure scheme S7 step 3. Yield: 234 mg of crude.

LCMS(ESI): [M]⁺ m/z: calcd 298.2; found 299.2; Rt=0.433 min.

### Step 1: Synthesis of 2-((2R,5S)-5-methyl-2-(2-((S)-1-methylpyrrolidin-3-yl)-2H-indazol-6-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S7 step 5. Yield: 65.4 mg (31.64%).

HPLC conditions: Column: SunFire C18 100*19 mm, 5 microM; 2-10 min 30-60% MeCN+FA, flow: 30 ml/min; (loading pump 4 ml/min MeCN).

LCMS(ESI): [M]⁺ m/z: calcd 616.2; found 617.2; Rt=1.043 min.

### Step 2: Synthesis of 2-((2R,5S)-5-methyl-2-(2-((S)-1-methylpyrrolidin-3-yl)-2H-indazol-6-yl)piperidin-1-yl)-2-oxo-N-(1-((2-(trimethylsilyl)ethoxy)methyl)-1H-pyrazolo[4,3-c]pyridin-7-yl)acetamide (Compound 117)

2-Oxo-2-[(2R,5S)-5-methyl-2-[2-[(3S)-1-methylpyrrolidin-3-yl]indazol-6-yl]-1-piperidyl]-N-[1-(2-trimethylsilylethoxymethyl)pyrazolo[4,3-c]pyridin-7-yl]acetamide (52.6 mg, 85.28 μmol) was dissolved in TFA (1 mL) and stirred overnight. The reaction mixture was concentrated in vacuum and purified by HPLC (2-10 min 30-60 MeCN+NH₃ 30 ml/min) to obtain 2-oxo-N-(1H-pyrazolo[4,3-c]pyridin-7-yl)-2-[(2R,5S)-5-methyl-2-[2-[(3S)-1-methylpyrrolidin-3-yl]indazol-6-yl]-1-piperidyl]acetamide (18.8 mg, 38.64 μmol, 45.31% yield).

Compound 117: ¹H NMR (600 MHz, DMSO-d₆) δ (ppm) 1.01-1.11 (m, 3H), 1.30-1.44 (m, 1H), 1.74-1.86 (m, 1H), 1.86-1.99 (m, 1H), 2.08-2.23 (m, 2H), 2.27-2.33 (m, 4H), 2.42-2.45 (m, 1H), 2.54-2.58 (m, 1H), 2.70-3.03 (m, 4H), 3.70-4.14 (m, 1H), 5.12-5.24 (m, 1H), 5.46-5.82 (m, 1H), 6.95-7.10 (m, 1H), 7.55 (s, 1H), 7.63-7.75 (m, 1H), 8.29-8.60 (m, 3H), 8.88-9.01 (m, 1H), 11.03-11.27 (m, 1H), 12.93-13.29 (m, 1H).

LCMS(ESI): [M]⁺ m/z: calcd 486.2; found 487.2; Rt=1.743 min.

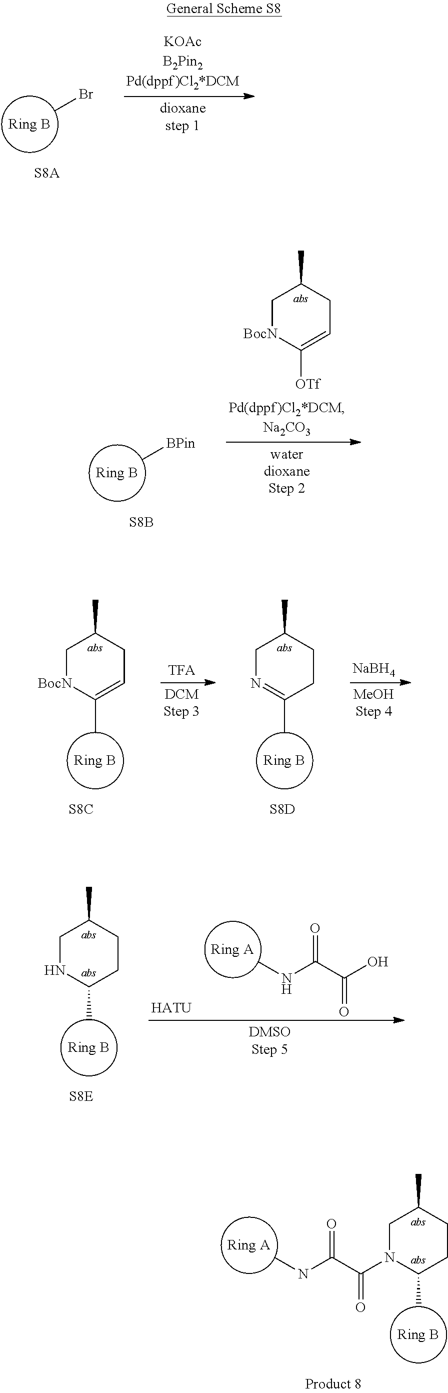

### Step 1: The Synthesis of S8B

S8A (1 eq), B₂Pin₂ (1.1 eq) and KOAc (2 eq) were mixed in dioxane. The resulting mixture was evacuated and then backfilled with argon, this operation was repeated three times, then Pd(dppf)Cl₂*DCM (0.05 eq) was added under argon. The reaction mixture was stirred under argon at 90° C. for 14 hr, then cooled and filtered. The filter cake was washed with dioxane twice. The solvent was evaporated to afford S8B.

### Step 2: The Synthesis of S8C

S8B (1 eq), tert-butyl (3S)-3-methyl-6-(trifluoromethylsulfonyloxy)-3,4-dihydro-2H-pyridine-1-carboxylate (1.2 eq), sodium carbonate (3 eq) were mixed together in dioxane-water mixture (3:1). The resulting mixture was evacuated and then backfilled with argon. This operation was repeated two times, then Pd(dppf)Cl₂*DCM (819.86 mg, 1.00 mmol) was added and the reaction mixture was stirred under argon at 90° C. overnight, then cooled down and concentrated in vacuum. The residue was diluted with MTBE and stirred for 0.5 hr. After the most of the residue had dissolved, anhydrous sodium sulphate was added, and the resulting mixture was filtered. The filter cake was additionally washed with MTBE (5*50 ml) and discarded. The filtrate was concentrated in vacuum to afford S8C.

### Step 3: The Synthesis of S8D

A solution of S8C (1.0 equiv) in TFA (50.0 mL) was stirred at 25° C. for 1 hr, and then concentrated in vacuum. Crushed ice (10 g) was added to the residue, and the resulting mixture was basified to pH 10 with 10% aqueous potassium carbonate solution and extracted with DCM (2*100.0 mL). The combined organic extracts were dried over sodium sulphate and concentrated under reduced pressure to afford S8D.

### Step 4: The Synthesis of S8E

S8D (1 eq) was dissolved in MeOH and the resulting solution was cooled to 0° C. in an ice bath. Sodium borohydride (2 eq) was added portion wise to the previous solution. After addition completed, the reaction mixture was allowed to warm to rt and stirred overnight. Water was added to the reaction mixture and the resulting mixture was concentrated in vacuum. The residue was diluted with water and the resulting mixture was extracted with DCM twice, dried over Na₂SO₄, filtered and evaporated to obtain S8E.

### Step 5A: Synthesis of Product 8

S8E (1 eq), oxamic acid (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain Product 8.

### Step 5B: Synthesis of Product 8

DIPEA (2.5 eq+1.0 eq per each acid eq, if amine salt used) was added to the solution of respective amine or it salt (S8E)(1 eq) and oxamic acid (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure product (Product 8).

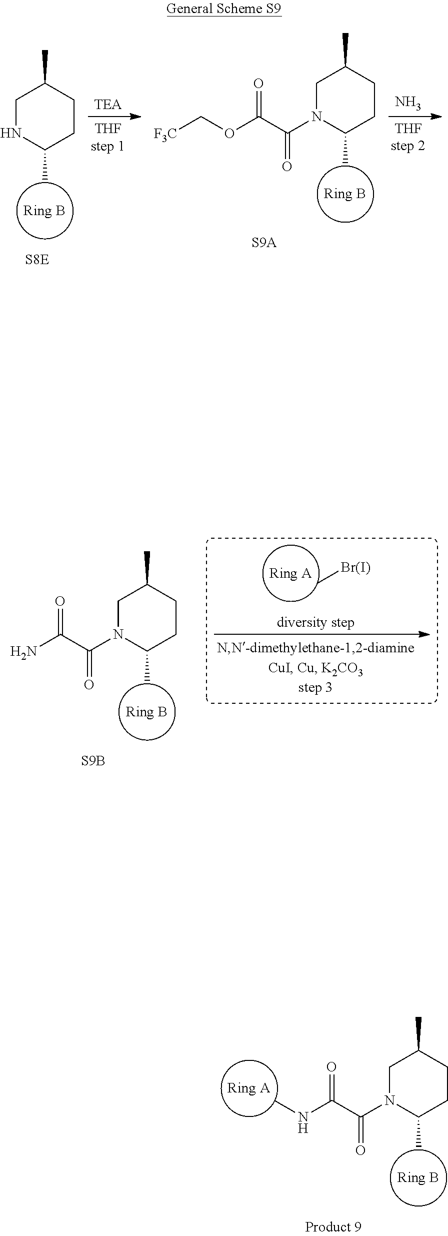

### Step 1: Synthesis of S9A

S8E was described in scheme S8.

S8E (1 eq) and TEA (1.1 eq) were dissolved in THF and cooled to 0° C., following by the dropwise addition of 2,2,2-trifluoroethyl 2-chloro-2-oxo-acetate (1.1 eq) under Ar and the reaction mixture was stirred for 12 hr at rt and evaporated under reduced pressure to give S9A which was used in the next step without further purification.

### Step 2: Synthesis of S9B

To a solution of S9A (1 eq) in THF (10 mL), ammonia (1 eq) was bubbled through for 10 min at 0° C. The reaction mixture was then stirred for 18 hr at rt. The reaction mixture was filtered off and the filtrate was evaporated in vacuum to give S9B which was used in the next step without further purification.

### Step 3A: Synthesis of Product 9

S9B (1 eq), RingABr (1.1 eq), Cu (1 eq), CuI (1 eq), Cs₂CO₃ (1.5 eq) and N,N-dimethylcyclohexane-1,2-diamine (1.5 eq) were mixed in dioxane under argon, and then stirred overnight at 95° C. for 24 hr in vial. The residue was purified by HPLC to obtain pure product (Product 9).

### Example 119. Synthesis of 2-((2R,5S)-5-methyl-2-(3-(((R)-1-methylpyrrolidin-2-yl) methoxy)phenyl)piperidin-1-yl)-2-oxo-N-(1H-pyrazolo[4,3-c]pyridin-7-yl)acetamide (Compound 118)

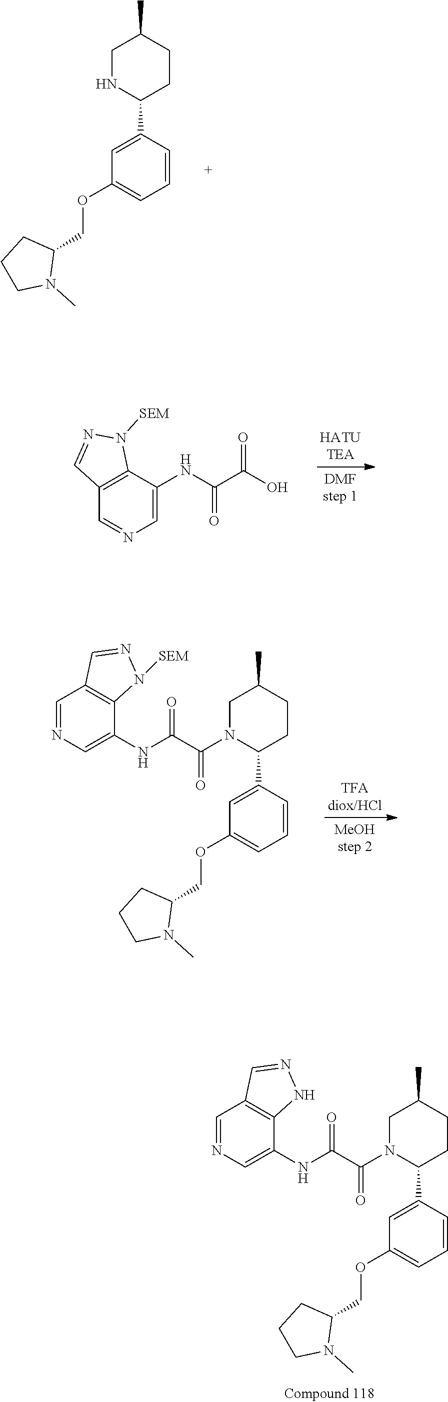 [SMILES: C[C@H]1CC[C@H](c2cccc(OC[C@H]3CCCN3C)c2)NC1 ; C[Si](C)(C)CCOCn1ncc2cncc(NC(=O)C(=O)O)c21 ; C[C@H]1CC[C@H](c2cccc(OC[C@H]3CCCN3C)c2)N(C(=O)C(=O)Nc2cncc3cnn(COCC[Si](C)(C)C)c23)C1 ; C[C@H]1CC[C@H](c2cccc(OC[C@H]3CCCN3C)c2)N(C(=O)C(=O)Nc2cncc3cn[nH]c23)C1]

The synthesis of (2R,5S)-5-methyl-2-(3-(((R)-1-methylpyrrolidin-2-yl) methoxy)phenyl)piperidine is prepared by general procedure scheme S8 step 4. Yield: 921 mg of crude.

LCMS(ESI): [M]⁺ m/z: calcd 288.2; found 289.2; Rt=0.675 min.

### Step 1: Synthesis of 2-((2R,5S)-5-methyl-2-(3-(((R)-1-methylpyrrolidin-2-yl) methoxy)phenyl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S8 step 5A. Yield: 38.3 mg (8.67%).

HPLC conditions: Column: SunFire C18 100*19 mm, 5 microM; 2-10 min 40-65% MeOH+FA, flow: 30 ml/min; (loading pump 4 ml/min MeOH).

LCMS(ESI): [M]⁺ m/z: calcd 606.2; found 607.2; Rt=1.247 min.

### Step 2: Synthesis of 2-((2R,5S)-5-methyl-2-(3-(((R)-1-methylpyrrolidin-2-yl) methoxy)phenyl)piperidin-1-yl)-2-oxo-N-(1H-pyrazolo[4,3-c]pyridin-7-yl)acetamide (Compound 118)

2-Oxo-2-[(2R,5S)-5-methyl-2-[[3-[(2R)-1-methylpyrrolidin-2-yl]methoxy]phenyl]-1-piperidyl]-N-[1-(2-trimethylsilylethoxymethyl)pyrazolo[4,3-c]pyridin-7-yl]acetamide (38.3 mg, 63.11 μmol) was dissolved in MeOH (3.94 mL) and hydrogen chloride solution 4.0M in dioxane (736.39 mg, 20.20 mmol, 920.49 μL) was added thereto. The resulting mixture was stirred overnight. The reaction mixture was concentrated in vacuum. The residue was dissolved in TFA (215.90 mg, 1.89 mmol, 145.88 μL) and stirred overnight. The reaction mixture was concentrated in vacuum and purified by HPLC (0-80% MeOH+NH₃ 30 ml/min) to obtain 2-oxo-N-(1H-pyrazolo[4,3-c]pyridin-7-yl)-2-[(2R,5S)-5-methyl-2-[3-[[(2R)-1-methylpyrrolidin-2-yl]methoxyphenyl]-1-piperidyl]acetamide (12.4 mg, 26.02 μmol, 41.23% yield).

Compound 118: ¹H NMR (600 MHz, DMSO-d₆) δ (ppm) 0.90-1.07 (m, 3H), 1.29-1.41 (m, 1H), 1.53-1.60 (m, 1H), 1.64-1.68 (m, 2H), 1.84-1.96 (m, 2H), 2.05-2.23 (m, 3H), 2.30-2.34 (m, 3H), 2.80-2.98 (m, 2H), 3.10-3.15 (m, 2H), 3.74-4.14 (m, 3H), 5.37-5.64 (m, 1H), 6.80-6.97 (m, 3H), 7.22-7.37 (m, 1H), 8.26-8.36 (m, 1H), 8.42-8.54 (m, 1H), 8.85-8.98 (m, 1H), 11.12 (br s, 1H), 13.07 (br s, 1H).

LCMS(ESI): [M]⁺ m/z: calcd 476.2; found 477.2; Rt=0.782 min.

### Example 120. N-(4-amino-1H-pyrazolo[4,3-c]pyridin-7-yl)-2-((2R,5S)-5-methyl-2-(4-(4-methylpiperazin-1-yl)phenyl)piperidin-1-yl)-2-oxoacetamide (Compound 120)

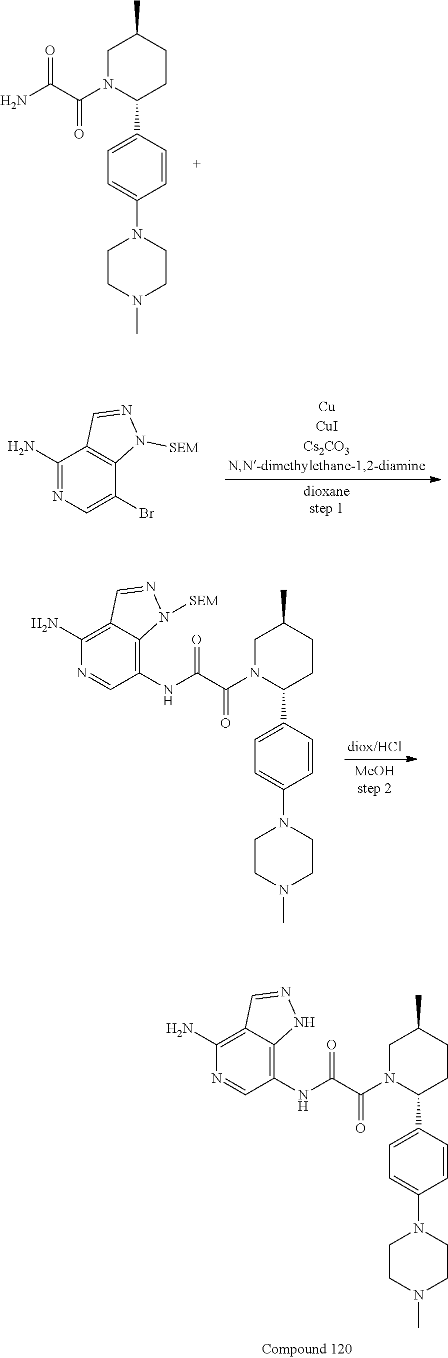 [SMILES: C[C@H]1CC[C@H](c2ccc(N3CCN(C)CC3)cc2)N(C(=O)C(N)=O)C1 ; C[Si](C)(C)CCOCn1ncc2c(N)ncc(Br)c21 ; C[C@H]1CC[C@H](c2ccc(N3CCN(C)CC3)cc2)N(C(=O)C(=O)Nc2cnc(N)c3cnn(COCC[Si](C)(C)C)c23)C1 ; C[C@H]1CC[C@H](c2ccc(N3CCN(C)CC3)cc2)N(C(=O)C(=O)Nc2cnc(N)c3cn[nH]c23)C1]

The Synthesis of 2-((2R,5S)-5-Methyl-2-(4-(4-methylpiperazin-1-yl)phenyl)piperidin-1-yl)-2-oxoacetamide is prepared by general procedure S1 Step 4B.

Yield: 0.6 g (49.64%)

LCMS(ESI): [M+H]⁺ m/z: calcd 345.2; found 345.2; Rt=0.754 min.

### Step 1: Synthesis of N-(4-amino-1-((2-(trimethylsilyl)ethoxy)methyl)-1H-pyrazolo[4,3-c]pyridin-7-yl)-2-((2R,5S)-5-methyl-2-(4-(4-methylpiperazin-1-yl)phenyl)piperidin-1-yl)-2-oxoacetamide

Prepared by general procedure scheme S9 step 3A. Yield: 144 mg (27.25%).

HPLC conditions: Column: XBridge BEH C18 100*19 mm, 5 microM; 0-1-6 min 20-20-70% water-MeCN+0.1% NH₄OH 30 ml/min; (loading pump 4 ml/min MeCN).

LCMS(ESI): [M]⁺ m/z: calcd 606.2; found 607.2; Rt=0.962 min.

### Step 2: Synthesis of N-(4-amino-1H-pyrazolo[4,3-c]pyridin-7-yl)-2-((2R,5S)-5-methyl-2-(4-(4-methylpiperazin-1-yl)phenyl)piperidin-1-yl)-2-oxoacetamide (Compound 120)

To the stirred solution of N-(4-amino-1-((2-(trimethylsilyl)ethoxy)methyl)-1H-pyrazolo[4,3-c]pyridin-7-yl)-2-((2R,5S)-5-methyl-2-(4-(4-methylpiperazin-1-yl)phenyl)piperidin-1-yl)-2-oxoacetamide (144 mg, 237.30 μmol) in MeOH (1 mL) hydrogen chloride solution 4.0M in dioxane (800.00 mg, 21.94 mmol, 1 mL) was added. The resulting mixture was stirred at 25° C. for 15 hr. The reaction mixture was concentrated under reduce pressure and was then purified by reverse phase HPLC (SYSTEM 0-0-25% 0-1-6 min H₂O/MeCN/0.1% FA, flow 30 ml/min (loading pump 4 ml/min MeCN) target mass 476 column: Chromatorex 18 SMB 100-ST 100*19 mm 5 um) to give N-(4-amino-1H-pyrazolo[4,3-c]pyridin-7-yl)-2-((2R,5S)-5-methyl-2-(4-(4-methylpiperazin-1-yl)phenyl)piperidin-1-yl)-2-oxoacetamide (9.5 mg, 16.21 μmol, 6.83% yield, ³HCl).

Compound 120: ¹H NMR (600 MHz, DMSO-d₆) δ (ppm) 0.66-1.03 (m, 3H), 1.06-1.27 (m, 1H), 1.28-1.53 (m, 1H), 1.56-2.07 (m, 3H), 2.08-2.29 (m, 4H), 2.41-2.45 (m, 4H), 3.04-3.13 (m, 4H), 3.66-4.23 (m, 1H), 4.87-5.64 (m, 1H), 6.07-7.32 (m, 6H), 7.49-7.77 (m, 1H), 8.14-8.26 (m, 1H), 9.55-10.51 (m, 1H), 12.56-13.37 (m, 1H).

LCMS(ESI): [M]⁺ m/z: calcd 476.2; found 477.2; Rt=1.525 min.

### Example 121. Synthesis of N-(4-amino-1H-pyrazolo[4,3-c]pyridin-7-yl)-2-((2R,5S)-5-methyl-2-(quinolin-7-yl)piperidin-1-yl)-2-oxoacetamide (Compound 121)

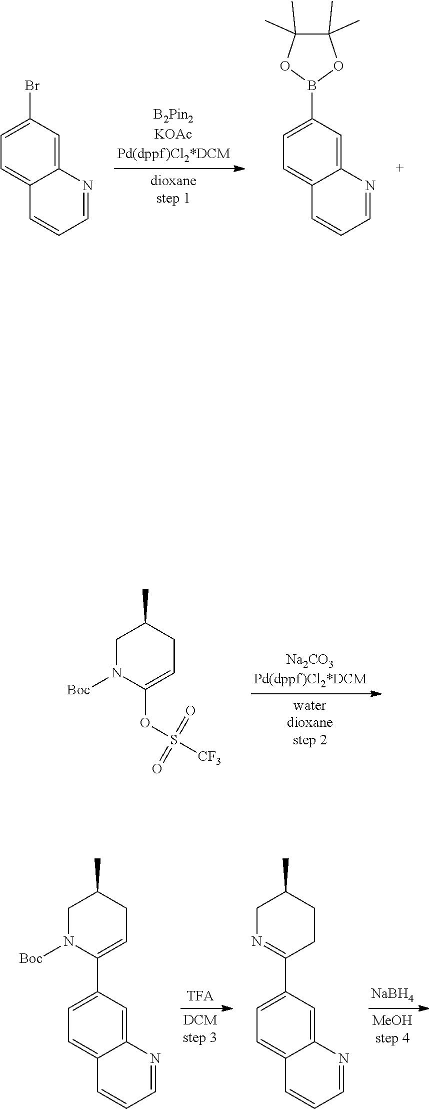 [SMILES: Brc1ccc2cccnc2c1 ; CC1(C)OB(c2ccc3cccnc3c2)OC1(C)C ; C[C@H]1CC=C(OS(=O)(=O)C(F)(F)F)N(C(=O)OC(C)(C)C)C1 ; C[C@H]1CC=C(c2ccc3cccnc3c2)N(C(=O)OC(C)(C)C)C1 ; C[C@H]1CCC(c2ccc3cccnc3c2)=NC1 ; "NaBH4"]

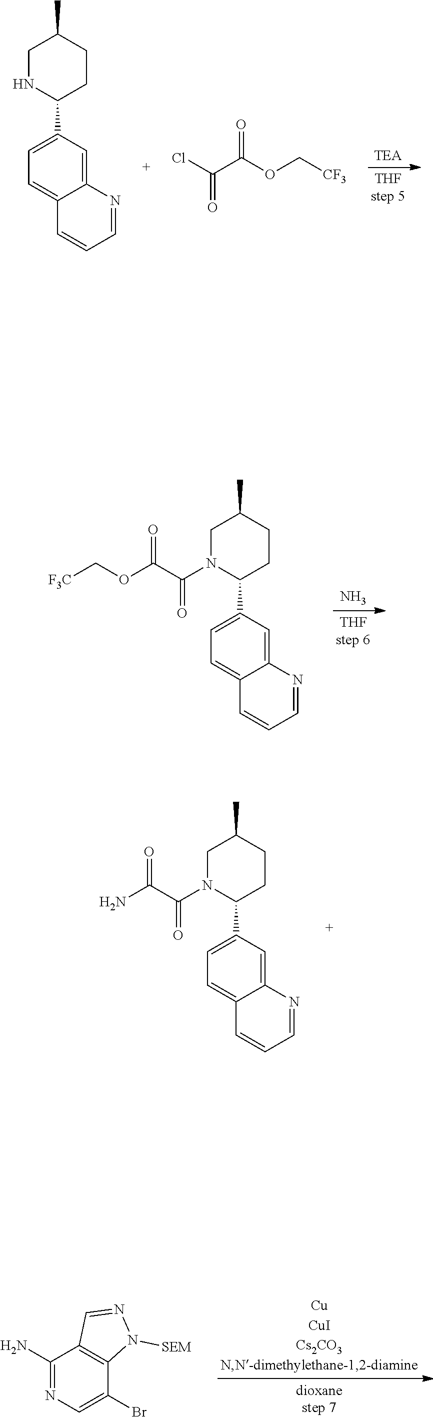

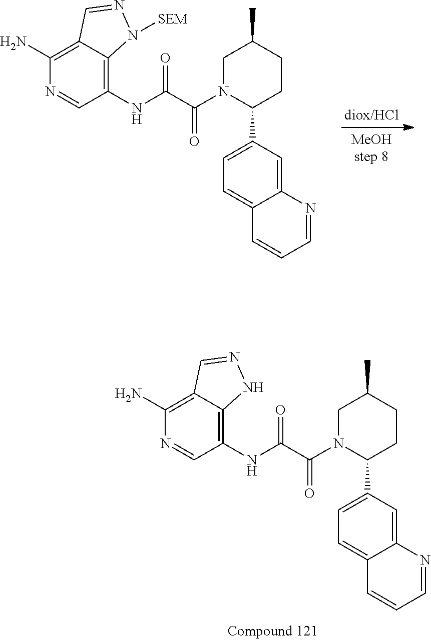 [SMILES: C[C@H]1CC[C@H](c2ccc3cccnc3c2)N(C(=O)C(=O)Nc2cnc(N)c3cnn(COCC[Si](C)(C)C)c23)C1 ; C[C@H]1CC[C@H](c2ccc3cccnc3c2)N(C(=O)C(=O)Nc2cnc(N)c3cn[nH]c23)C1]

### Step 1: Synthesis of 7-(4,4,5,5-tetramethyl-1,3,2-dioxaborolan-2-yl) quinoline

Prepared by general procedure scheme S8 step 1. Yield: 3.6 g of crude.

LCMS(ESI): [M]⁺ m/z: calcd 255.2; found 256.2; Rt=1.051 min.

### Step 2: Synthesis of(S)-tert-butyl 3-methyl-6-(quinolin-7-yl)-3,4-dihydropyridine-1(2H)-carboxylate

Prepared by general procedure scheme S8 step 2. Yield: 4.5 g of crude.

LCMS(ESI): [M]⁺ m/z: calcd 324.2; found 325.2; Rt=3.513 min.

### Step 3: Synthesis of(S)-7-(5-methyl-3,4,5,6-tetrahydropyridin-2-yl) quinoline

Prepared by general procedure scheme S8 step 3. Yield: 3 g of crude.

LCMS(ESI): [M]⁺ m/z: calcd 224.2; found 225.2; Rt=0.765 min.

### Step 4: Synthesis of 7-((2R,5S)-5-methylpiperidin-2-yl) quinoline

Prepared by general procedure scheme S8 step 4. Yield: 2.3 g (75.98%).

LCMS(ESI): [M]⁺ m/z: calcd 226.2; found 227.2; Rt=0.760 min.

### Step 5: Synthesis of 2,2,2-trifluoroethyl 2-((2R,5S)-5-methyl-2-(quinolin-7-yl)piperidin-1-yl)-2-oxoacetate

Prepared by general procedure scheme S9 step 1. Yield: 0.8 g of crude.

LCMS(ESI): [M]⁺ m/z: calcd 380.2; found 381.2; Rt=1.221 min.

### Step 6: Synthesis of 2-((2R,5S)-5-methyl-2-(quinolin-7-yl)piperidin-1-yl)-2-oxoacetamide

Prepared by general procedure scheme S9 step 2. Yield: 0.6 g of crude.

LCMS(ESI): [M]⁺ m/z: calcd 297.2; found 298.2; Rt=1.815 min.

### Step 7: Synthesis of N-(4-amino-1-((2-(trimethylsilyl)ethoxy)methyl)-1H-pyrazolo[4,3-c]pyridin-7-yl)-2-((2R,5S)-5-methyl-2-(quinolin-7-yl)piperidin-1-yl)-2-oxoacetamide

Prepared by general procedure scheme S9 step 3A. Yield: 0.47 g of crude.

LCMS(ESI): [M]⁺ m/z: calcd 559.2; found 560.2; Rt=3.398 min.

### Step 8: Synthesis of N-(4-amino-1H-pyrazolo[4,3-c]pyridin-7-yl)-2-((2R,5S)-5-methyl-2-(quinolin-7-yl)piperidin-1-yl)-2-oxoacetamide (Compound 121)

Hydrogen chloride solution 4.0M in dioxane (6.12 g, 167.94 mmol, 7.65 mL) was added to a solution of N-[4-amino-1-(2-trimethylsilylethoxymethyl)pyrazolo[4,3-c]pyridin-7-yl]-2-oxo-2-[(2R,5S)-5-methyl-2-(7-quinolyl)-1-piperidyl]acetamide (0.94 g, 1.68 mmol) in MeOH (10 mL) and stirred at 25° C. for 14 hr. Reaction mixture was concentrated, triturate with IPA (10 mL), filtered washed with IPA (5 mL) and MTBE (10 mL) and resulting brown solid purified by HPLC (column: XBridge BEH C18 100×19 mm, 5 um; mobile phase: 10-35% 0-5 min H₂O/MeCN/0.1% NH₄OH, flow rate: 30 ml/min) and chiral HPLC to give pure N-(4-amino-1H-pyrazolo[4,3-c]pyridin-7-yl)-2-oxo-2-[(2R,5S)-5-methyl-2-(7-quinolyl)-1-piperidyl]acetamide (0.162 g, 377.21 μmol, 22.46% yield).

Compound 121: ¹H NMR (600 MHZ, DMSO-d₆) δ (ppm) 0.71-1.10 (m, 3H), 1.36-1.63 (m, 1H), 1.69-1.82 (m, 1H), 1.86-1.97 (m, 1H), 2.13-2.44 (m, 2H), 2.64-3.25 (m, 1H), 3.48-4.19 (m, 1H), 5.20-5.98 (m, 1H), 6.57-6.99 (m, 2H), 7.48-7.63 (m, 2H), 7.65-7.80 (m, 1H), 7.93-8.08 (m, 2H), 8.12-8.27 (m, 1H), 8.29-8.39 (m, 1H), 8.82-8.95 (m, 1H), 10.50-10.63 (m, 1H), 12.56-13.01 (m, 1H).

LCMS(ESI): [M]⁺ m/z: calcd 429.2; found 430.2; Rt=1.987 min.

### Example 122. Synthesis of N-(4-amino-1H-pyrazolo[4,3-c]pyridin-7-yl)-2-oxo-2-[(2R,5S)-5-methyl-2-(2-methylindazol-5-yl)-1-piperidyl]acetamide (Compound 124)

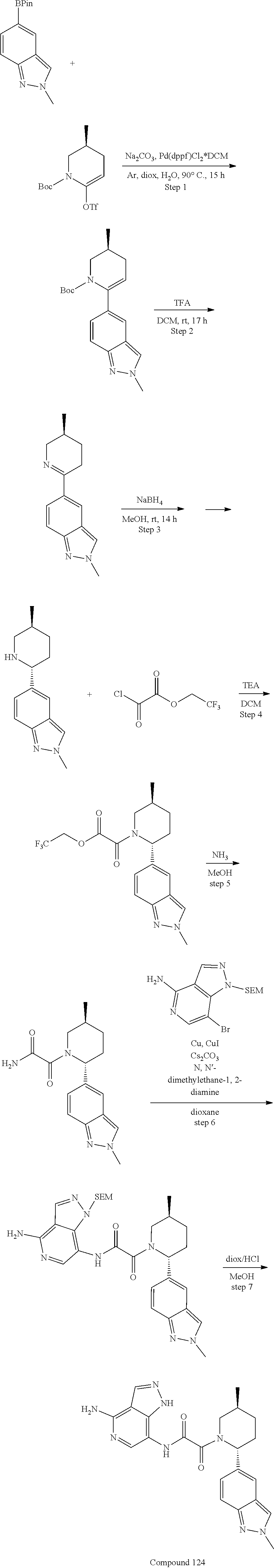

### Step 1: The Synthesis of tert-butyl (3S)-3-methyl-6-(2-methyllindazol-5-yl)-3,4-dihydro-2H-pyridine-1-carboxylate

To a stirred solution of 2-methyl-5-(4,4,5,5-tetramethyl-1,3,2-dioxaborolan-2-yl)indazole (1 g, 3.87 mmol) and tert-butyl (3S)-3-methyl-6-(trifluoromethylsulfonyloxy)-3,4-dihydro-2H-pyridine-1-carboxylate (1.5 g, 4.34 mmol) in DiOX (15 mL) and Water (5 mL) was added Sodium carbonate (0.9 g, 8.49 mmol, 355.45 μL). The resulting suspension was degassed with argon at 50° C. for 0.5 h. Pd(dppf)Cl₂ (0.15 g, 183.69 μmol) was added. The reaction mixture was stirred at 80° C. for 16 h. Upon completion, the reaction mixture was concentrated under reduced pressure, the crude product was dissolved in CHCl₃. The mixture was dried over Na₂SO₄, filtered and the filtrate was evaporated. The reaction was successful. The desired product tert-butyl (3S)-3-methyl-6-(2-methylindazol-5-yl)-3,4-dihydro-2H-pyridine-1-carboxylate (0.9 g, 2.75 mmol, 70.95% yield) was isolated.

### Step 2: The Synthesis of 2-methyl-5-[(3S)-3-methyl-2,3,4,5-tetrahydropyridin-6-yl]indazole

The stirred solution of tert-butyl (3S)-3-methyl-6-(2-methylindazol-5-yl)-3,4-dihydro-2H-pyridine-1-carboxylate (0.9 g, 2.75 mmol) in MeOH (10 mL) and DiOX/HCl (5 mL) was allowed to stir at 25° C. for 16 h. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The reaction was successful. The desired product 2-methyl-5-[(3S)-3-methyl-2,3,4,5-tetrahydropyridin-6-yl]indazole (0.6 g, 2.64 mmol, 96.03% yield) was isolated.

LCMS(ESI): [M+H]⁺ m/z: calcd 227.3; found 228.4; Rt=0.775 min.

### Step 3: The Synthesis of 2-methyl-5-[(2R,5S)-5-methyl-2-piperidyl]indazole

To a stirred solution of 2-methyl-5-[(3S)-3-methyl-2,3,4,5-tetrahydropyridin-6-yl]indazole (0.6 g, 2.64 mmol) in MeOH (20 mL) was added Sodium Borohydride (0.15 g, 3.96 mmol, 139.66 μL) at 5° C. The resulting reaction mixture was stirred at 25° C. for 16 h. Upon completion, the reaction mixture was evaporated, quenched with water (20 mL). The aqueous phase was extracted with CHCl₃ (2*20 mL). The combined organic phase was dried over Na₂SO₄ and concentrated under reduced pressure. The reaction was successful. The desired product 2-methyl-5-[(2R,5S)-5-methyl-2-piperidyl]indazole (0.6 g, 2.62 mmol, 99.12% yield) was isolated.

LCMS(ESI): [M+H]⁺ m/z: calcd 229.2; found 230.2; Rt=0.793 min.

### Step 4: The Synthesis of 2,2,2-trifluoroethyl 2-oxo-2-[(2R,5S)-5-methyl-2-(2-methylindazol-5-yl)-1-piperidyl]acetate

To a solution of 2-methyl-5-[(2R,5S)-5-methyl-2-piperidyl]indazole (0.6 g, 2.62 mmol) and Triethylamine (508.20 mg, 5.02 mmol, 0.7 mL) in CHCl₃ (15 mL) was added 2,2,2-trifluoroethyl 2-chloro-2-oxo-acetate (0.6 g, 3.15 mmol) at 5° C. The resulting reaction mixture was allowed to warm to room temperature and stirred for 16 h at 25° C. After 16 hr the reaction mixture was quenched with water (20 ml) and extracted with CHCl₃ (2*10 ml). The organic layer was dried over Na₂SO₄ and evaporated under reduced pressure. The reaction was successful. The desired product 2,2,2-trifluoroethyl 2-oxo-2-[(2R,5S)-5-methyl-2-(2-methylindazol-5-yl)-1-piperidyl]acetate (0.9 g, 2.35 mmol, 89.73% yield) was isolated.

LCMS(ESI): [M+H]⁺ m/z: calcd 383.1; found 384.2; Rt=1.256 min.

### Step 5: The Synthesis of 2-oxo-2-[(2R,5S)-5-methyl-2-(2-methylindazol-5-yl)-1-piperidyl]acetamide

To the solution of 2,2,2-trifluoroethyl 2-oxo-2-[(2R,5S)-5-methyl-2-(2-methylindazol-5-yl)-1-piperidyl]acetate (0.9 g, 2.35 mmol) in MeOH (10 mL) was added MeOH/NH₃ (10 mL). The resulting reaction mixture was stirred at room temperature for 16 h. After 16 hr the reaction mixture was evaporated under reduced pressure. The reaction was successful. The desired product 2-oxo-2-[(2R,5S)-5-methyl-2-(2-methylindazol-5-yl)-1-piperidyl]acetamide (0.7 g, 2.33 mmol, 99.27% yield) was isolated.

LCMS(ESI): [M+H]⁺ m/z: calcd 300.1; found 301.2; Rt=1.005 min.

### Step 6: The Synthesis of N-[4-amino-1-(2-trimethylsilylethoxymethyl)pyrazolo[4,3-c]pyridin-7-yl]-2-oxo-2-[(2R,5S)-5-methyl-2-(2-methylindazol-5-yl)-1-piperidyl]acetamide

2-oxo-2-[(2R,5S)-5-methyl-2-(2-methylindazol-5-yl)-1-piperidyl]acetamide (0.15 g, 499.41 μmol), 7-bromo-1-(2-trimethylsilylethoxymethyl)pyrazolo[4,3-c]pyridin-4-amine (0.2 g, 582.59 μmol), Copper (I) iodide (0.1 g, 525.07 μmol, 17.79 μL), Cu (0.03 g, 472.07 μmol) and Cesium carbonate (0.3 g, 920.75 μmol) were mixed together in DiOX (5 mL). The resulting suspension was degassed with argon at 25° C. for 0.1 h. (1R,2R)—N1,N2-dimethylcyclohexane-1,2-diamine (158.00 mg, 1.11 mmol, 0.2 mL) was added thereto and the resulting mixture was stirred for 16 h at 90° C. After completion the reaction mixture was filtered and the filtrate was concentrated in vacuum. The obtained crude product was purified by reverse phase HPLC chromatography (Device (Mobile Phase, Column): SYSTEM 40-90% 0-5 min H₂O/MeOH/0.1% NH₄OH, flow: 30 ml/min (loading pump 4 ml/min methanol) target mass 562.75 column: XBridge BEH C18 100×19 mm, 5 um) to afford N-[4-amino-1-(2-trimethylsilylethoxymethyl)pyrazolo[4,3-c]pyridin-7-yl]-2-oxo-2-[(2R,5S)-5-methyl-2-(2-methylindazol-5-yl)-1-piperidyl]acetamide (79 mg, 140.39 μmol, 28.11% yield). The reaction was successful. The desired product N-[4-amino-1-(2-trimethylsilylethoxymethyl)pyrazolo[4,3-c]pyridin-7-yl]-2-oxo-2-[(2R,5S)-5-methyl-2-(2-methylindazol-5-yl)-1-piperidyl]acetamide (79 mg, 140.39 μmol, 28.11% yield) was isolated.

LCMS(ESI): [M+H]⁺ m/z: calcd 562.3; found 563.4; Rt=1.224 min.

### Step 7: The Synthesis of N-(4-amino-1H-pyrazolo[4,3-c]pyridin-7-yl)-2-oxo-2-[(2R,5S)-5-methyl-2-(2-methylindazol-5-yl)-1-piperidyl]acetamide (Compound 124)

2-oxo-2-[(2R,5S)-5-methyl-2-(2-methylindazol-5-yl)-1-piperidyl]acetamide (0.15 g, 499.41 μmol), 7-bromo-1-(2-trimethylsilylethoxymethyl)pyrazolo[4,3-c]pyridin-4-amine (0.2 g, 582.59 μmol), Copper (I) iodide (0.1 g, 525.07 μmol, 17.79 μL), Cu (0.03 g, 472.07 μmol) and Cesium carbonate (0.3 g, 920.75 μmol) were mixed together in DiOX (5 mL). The resulting suspension was degassed with argon at 25° C. for 0.1 h. (1R,2R)—N1,N2-dimethylcyclohexane-1,2-diamine (158.00 mg, 1.11 mmol, 0.2 mL) was added thereto and the resulting mixture was stirred for 16 h at 90° C. After completion the reaction mixture was filtered and the filtrate was concentrated in vacuum. The obtained crude product was purified by reverse phase HPLC chromatography (Device (Mobile Phase, Column): SYSTEM 40-90% 0-5 min H₂O/MeOH/0.1% NH₄OH, flow: 30 ml/min (loading pump 4 ml/min methanol) target mass 562.75 column: XBridge BEH C18 100×19 mm, 5 um) to afford N-[4-amino-1-(2-trimethylsilylethoxymethyl)pyrazolo[4,3-c]pyridin-7-yl]-2-oxo-2-[(2R,5S)-5-methyl-2-(2-methylindazol-5-yl)-1-piperidyl]acetamide (79 mg, 140.39 μmol, 28.11% yield). The reaction was successful. The desired product N-[4-amino-1-(2-trimethylsilylethoxymethyl)pyrazolo[4,3-c]pyridin-7-yl]-2-oxo-2-[(2R,5S)-5-methyl-2-(2-methylindazol-5-yl)-1-piperidyl]acetamide (79 mg, 140.39 μmol, 28.11% yield) was isolated.

LCMS(ESI): [M+H]⁺ m/z: calcd 432.2; found 433.2; Rt=2.253 min.

### Example 123. Synthesis of N-(4-amino-1H-pyrazolo[4,3-c]pyridin-7-yl)-2-(2-cyclopentylpiperidin-1-yl)-2-oxoacetamide (Compound 127)

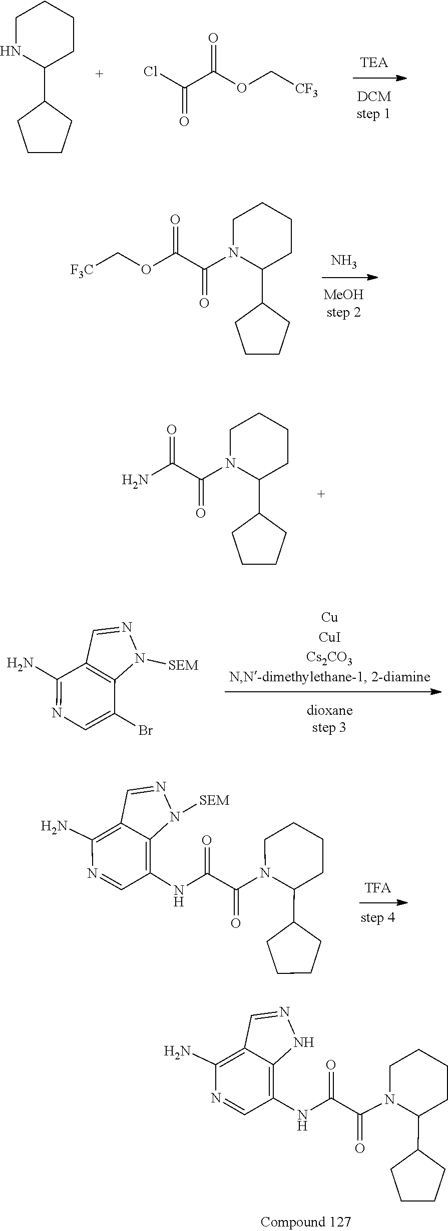

### Step 1: Synthesis of 2,2,2-trifluoroethyl 2-(2-cyclopentylpiperidin-1-yl)-2-oxoacetate

Prepared by general procedure scheme S9 step 1. Yield: 1.07 g of crude.

¹H NMR (400 MHZ, CDCl₃) δ (ppm) 1.03 (m, 1H), 1.21 (m, 2H), 1.69 (m, 9H), 2.05 (m, 1H), 2.42 (m, 1H), 2.78 (m, 1H), 3.20 (m, 2H), 4.35 (m, 1H), 4.66 (m, 2H).

### Step 2: Synthesis of 2-(2-cyclopentylpiperidin-1-yl)-2-oxoacetamide

2,2,2-Trifluoroethyl 2-(2-cyclopentyl-1-piperidyl)-2-oxo-acetate (1.07 g, 3.48 mmol) was dissolved in MeOH/NH₃ (40 mL) as stirred overnight at 20° C. The reaction mixture was evaporated to dryness. The obtained product was used in the next step without further purification. 2-(2-Cyclopentyl-1-piperidyl)-2-oxo-acetamide (0.9 g, crude) was obtained as an orange gum.

LCMS(ESI): [M]⁺ m/z: calcd 224.2; found 225.2; Rt=0.909 min.

### Step 3: Synthesis of N-(4-amino-1-((2-(trimethylsilyl)ethoxy)methyl)-1H-pyrazolo[4,3-c]pyridin-7-yl)-2-(2-cyclopentylpiperidin-1-yl)-2-oxoacetamide

Prepared by general procedure scheme S9 step 3A. Yield: 92 mg (21.20%).

HPLC conditions: Column: SunFire C18 100*19 mm, 5 microM; 2-10 min 10-50% MeCN+FA 30 ml/min; (loading pump 4 ml/min MeCN+FA).

LCMS(ESI): [M]⁺ m/z: calcd 486.2; found 487.2; Rt=1.438 min.

### Step 4: Synthesis of N-(4-amino-1H-pyrazolo[4,3-c]pyridin-7-yl)-2-(2-cyclopentylpiperidin-1-yl)-2-oxoacetamide (Compound 127)

N-[4-Amino-1-(2-trimethylsilylethoxymethyl)pyrazolo[4,3-c]pyridin-7-yl]-2-(2-cyclopentyl-1-piperidyl)-2-oxo-acetamide (0.092 g, 189.04 μmol) was dissolved in TFA (646.63 mg, 5.67 mmol, 436.91 μL) and stirred at 20° C. for 3 hr. The reaction mixture was submitted to HPLC (2-10 min 30-65% MeOH+NH₃ 30/min; loading pump 4 ml/min MeOH+NH₃ column SunFire 19*100 mm). N-(4-Amino-1H-pyrazolo[4,3-c]pyridin-7-yl)-2-(2-cyclopentyl-1-piperidyl)-2-oxo-acetamide (0.018 g, 50.50 μmol, 26.72% yield) was obtained as a light-yellow solid.

Compound 127: ¹H NMR (600 MHZ, DMSO-d₆) δ (ppm) 1.18 (m, 4H), 1.61 (m, 11H), 2.86 (m, 1H), 3.90 (m, 2H), 6.64 (m, 2H), 7.72 (m, 1H), 8.14 (m, 1H), 10.29 (m, 1H), 12.66 (m, 1H).

LCMS(ESI): [M]⁺ m/z: calcd 356.2; found 357.2; Rt=1.962 min.

### Example 124. Synthesis of 2-((2R,5S)-2-(2-(rac-(3S,4R)-3-methoxy-1-methylpiperidin-4-yl)benzo[d]thiazol-5-yl)-5-methylpiperidin-1-yl)-2-oxo-N-(1H-pyrazolo[4,3-c]pyridin-7-yl)acetamide (Compound 128)

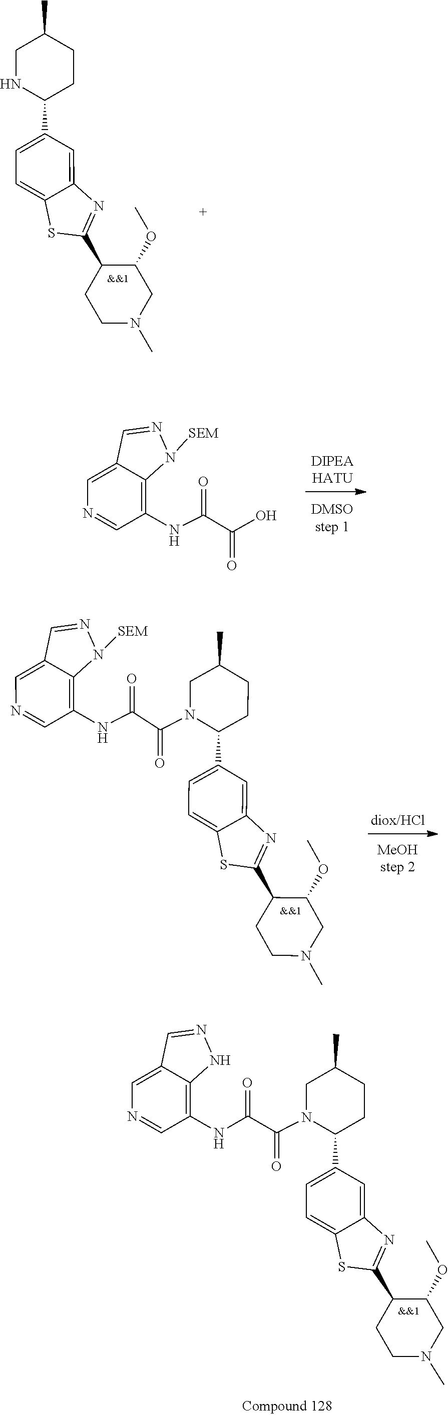 [SMILES: CO[C@@H]1CN(C)CC[C@H]1c1nc2cc([C@H]3CC[C@H](C)CN3)ccc2s1 ; C[Si](C)(C)CCOCn1ncc2cncc(NC(=O)C(=O)O)c21 ; CO[C@@H]1CN(C)CC[C@H]1c1nc2cc([C@H]3CC[C@H](C)CN3C(=O)C(=O)Nc3cncc4cnn(COCC[Si](C)(C)C)c34)ccc2s1 ; CO[C@@H]1CN(C)CC[C@H]1c1nc2cc([C@H]3CC[C@H](C)CN3C(=O)C(=O)Nc3cncc4cn[nH]c34)ccc2s1]

The synthesis of 2-(rac-(3S,4R)-3-methoxy-1-methylpiperidin-4-yl)-5-((2R,5S)-5-methylpiperidin-2-yl)benzo[d]thiazole is prepared by general procedure scheme S2 step 5. Yield: 340 mg of crude.

LCMS(ESI): [M]⁺ m/z: calcd 359.2; found 360.2; Rt=0.721 min.

### Step 1: Synthesis of 2-((2R,5S)-2-(2-(rac-(3S,4R)-3-methoxy-1-methylpiperidin-4-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6B. Yield: 98.5 mg (26.11%).

HPLC conditions: Column: SunFire C18 100*19 mm, 5 microM; 2-10 min 30-50% MeCN+FA; (loading pump 4 ml/min MeCN).

LCMS(ESI): [M]⁺ m/z: calcd 678.2; found 679.2; Rt=1.280 min.

### Step 2: Synthesis of 2-((2R,5S)-2-(2-(rac-(3S,4R)-3-methoxy-1-methylpiperidin-4-yl)benzo[d]thiazol-5-yl)-5-methylpiperidin-1-yl)-2-oxo-N-(1H-pyrazolo[4,3-c]pyridin-7-yl)acetamide (Compound 128)

2-Oxo-2-[(2R,5S)-5-methyl-2-[2-[rac-(3S,4R)-3-methoxy-1-methyl-4-piperidyl]-1,3-benzothiazol-5-yl]-1-piperidyl]-N-[1-(2-trimethylsilylethoxymethyl)pyrazolo[4,3-c]pyridin-7-yl]acetamide (126.08 mg, 185.98 μmol) was dissolved in MeOH (1 mL) and diox/HCl (1 g, 3.72 mmol, 1 mL) was added thereto. Then it was stirred at rt for 2 hr. The reaction mixture was evaporated. Crude product was purified by HPLC to afford 2-oxo-N-(1H-pyrazolo[4,3-c]pyridin-7-yl)-2-[(2R,5S)-5-methyl-2-[2-[rac-(3S,4R)-3-methoxy-1-methyl-4-piperidyl]-1,3-benzothiazol-5-yl]-1-piperidyl]acetamide (0.01 g, 18.26 μmol, 9.82% yield).

Compound 128: ¹H NMR (600 MHz, DMSO-d₆) δ 1.05-1.10 (m, 3H), 1.34-1.44 (m, 1H), 1.71-1.98 (m, 3H), 2.03-2.36 (m, 6H), 2.61-3.13 (m, 6H), 3.35-3.59 (m, 4H), 3.77-4.15 (m, 1H), 5.59-5.79 (m, 1H), 7.39-7.50 (m, 1H), 7.87-8.11 (m, 2H), 8.29-8.38 (m, 1H), 8.40-8.58 (m, 1H), 8.87-9.00 (m, 1H), 11.04-11.20 (m, 1H), 12.95-13.10 (m, 1H).

LCMS(ESI): [M]⁺ m/z: calcd 547.2; found 548.2; Rt=0.702 min.

### Example 125. Synthesis of 2-oxo-N-(1H-pyrazolo[4,3-c]pyridin-7-yl)-2-[(2R,5S)-5-methyl-2-(2-tetrahydropyran-4-ylindazol-6-yl)-1-piperidyl]acetamide (Compound 130)

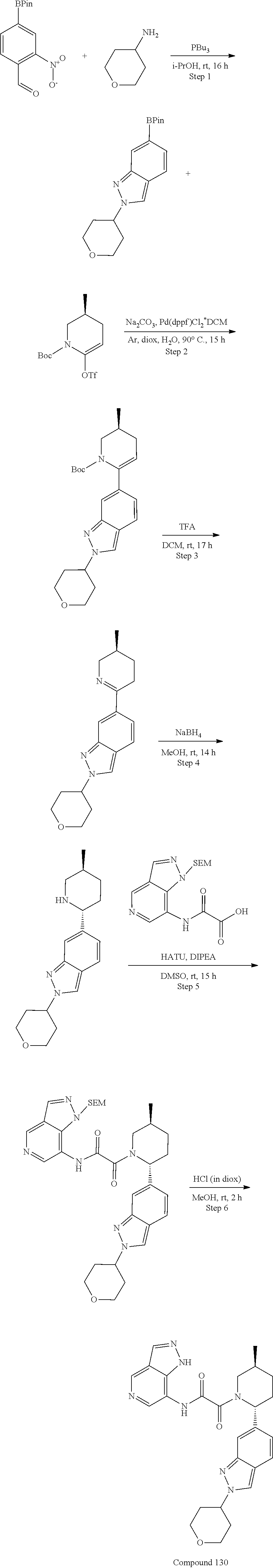

### Step 1: The Synthesis of 2-tetrahydropyran-4-yl-6-(4,4,5,5-tetramethyl-1,3,2-dioxaborolan-2-yl)indazole

### Prepared by General Procedure Scheme S7 Step 1

FCC conditions: FCC (gradient EtOAc in hexane from 0% to 100%)

Yield: 1.64 g (69.39%)

LCMS(ESI): [M+2H]⁺ m/z: calcd 328.2; found 329.0; Rt=1.168 min.

### Step 2: The Synthesis of tert-butyl (3S)-3-methyl-6-(2-tetrahydropyran-4-ylindazol-6-yl)-3,4-dihydro-2H-pyridine-1-carboxylate

### Prepared by General Procedure Scheme S7 Step 2

Yield: 2.07 g (crude)

LCMS(ESI): [M+H]⁺ m/z: calcd 397.2; found 398.2; Rt=1.352 min.

### Step 3: The Synthesis of 6-[(3S)-3-methyl-2,3,4,5-tetrahydropyridin-6-yl]-2-tetrahydropyran-4-yl-indazole

### Prepared by General Procedure Scheme S7 Step 3

Yield: 842 mg (54.3%)

LCMS(ESI): [M+H]⁺ m/z: calcd 297.2; found 298.2; Rt=0.732 min.

### Step 4: The Synthesis of 6-[(2R,5S)-5-methyl-2-piperidyl]-2-tetrahydropyran-4-yl-indazole

### Prepared by General Procedure Scheme S7 Step 4 (Method B)

Yield: 832 mg (98.15%)

LCMS(ESI): [M+H]⁺ m/z: calcd 299.2; found 300.2; Rt=0.854 min.

### Step 5: The Synthesis of 2-((2R,5S)-5-methyl-2-(2-(tetrahydro-2H-pyran-4-yl)-2H-indazol-6-yl)piperidin-1-yl)-2-oxo-N-(1-((2-(trimethylsilyl)ethoxy)methyl)-1H-pyrazolo[4,3-c]pyridin-7-yl)acetamide

### Prepared by General Procedure Scheme S7 Step 5

HPLC conditions: 2-10 min 30-60 MeOH+formic acid 30 ml/min.

Yield: 150.4 mg (36.44%)

LCMS(ESI): [M+H]⁺ m/z: calcd 617.4; found 618.4; Rt=1.353 min.

### Step 6: Synthesis of 2-oxo-N-(1H-pyrazolo[4,3-c]pyridin-7-yl)-2-[(2R,5S)-5-methyl-2-(2-tetrahydropyran-4-ylindazol-6-yl)-1-piperidyl]acetamide (Compound 130)

2-oxo-2-[(2R,5S)-5-methyl-2-(2-tetrahydropyran-4-ylindazol-6-yl)-1-piperidyl]-N-[1-(2-trimethylsilylethoxymethyl)pyrazolo[4,3-c]pyridin-7-yl]acetamide (150.4 mg, 243.44 μmol) was dissolved in TFA (1 mL) and the resulting mixture was stirred overnight. The reaction mixture was concentrated in vacuo and purified by HPLC (2-10 min 30-60 MeOH+NH3) to obtain 2-oxo-N-(1H-pyrazolo[4,3-c]pyridin-7-yl)-2-[(2R,5S)-5-methyl-2-(2-tetrahydropyran-4-ylindazol-6-yl)-1-piperidyl]acetamide (24.5 mg, 50.25 μmol, 20.64% yield) and 2-oxo-N-(1H-pyrazolo[4,3-c]pyridin-7-yl)-2-[(2R,5S)-5-methyl-2-(2-tetrahydropyran-4-ylindazol-6-yl)-1-piperidyl]acetamide (48.5 mg, 99.48 μmol, 40.86% yield).

¹H NMR (600 MHz, dmso) δ 0.85-1.10 (m, 3H), 1.28-1.44 (m, 1H), 1.75-1.86 (m, 1H), 1.86-1.99 (m, 1H), 2.01-2.18 (m, 5H), 2.20-2.32 (m, 1H), 2.87-2.92 (m, 0.3H), 3.34-3.38 (m, 0.7H), 3.45-3.54 (m, 2H), 3.76-4.14 (m, 3H), 4.68-4.81 (m, 1H), 5.43-5.82 (m, 1H), 6.97-7.13 (m, 1H), 7.55-7.60 (m, 1H), 7.65-7.75 (m, 1H), 8.25-8.43 (m, 2H), 8.43-8.58 (m, 1H), 8.87-8.99 (m, 1H), 10.95-11.29 (m, 1H), 12.88-13.29 (m, 1H).

LCMS(ESI): [M+H]⁺ m/z: calcd 487.2; found 488.4; Rt=2.223 min.

### Example 126. 2-((2R,5S)-2-(2-(1-cyclopropylpiperidin-4-yl)benzo[d]thiazol-5-yl)-5-methylpiperidin-1-yl)-2-oxo-N-(1H-pyrazolo[4,3-c]pyridin-7-yl)acetamide (Compound 131)

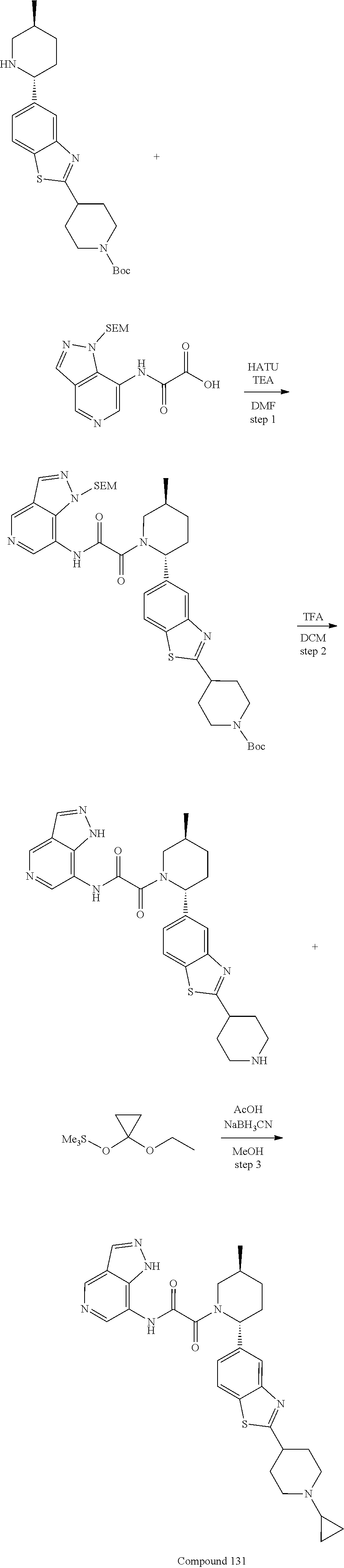

The synthesis of 2-(1-cyclopropylpiperidin-4-yl)-5-((2R,5S)-5-methylpiperidin-2-yl)benzo[d]thiazole is given by Intermediate 3.

### Step 1: Synthesis of tert-butyl 4-(5-((2R,5S)-5-methyl-1-(2-oxo-2-((1-((2-(trimethylsilyl)ethoxy)methyl)-1H-pyrazolo[4,3-c]pyridin-7-yl)amino) acetyl)piperidin-2-yl)benzo[d]thiazol-2-yl)piperidine-1-carboxylate

Prepared by general procedure scheme S2 step 6A. Yield: 500 mg (94.37%).

HPLC conditions: Column: Chromatorex 18 SMB100-5T 100*19 mm, 5 microM; 0-1-6 min 65-65-100% water-MeCN; (loading pump 4 ml/min MeCN).

LCMS(ESI): [M]⁺ m/z: calcd 733.2; found 734.2; Rt=1.834 min.

### Step 2: Synthesis of 2-((2R,5S)-5-methyl-2-(2-(piperidin-4-yl)benzo[d]thiazol-5-yl)piperidin-1-yl)-2-oxo-N-(1H-pyrazolo[4,3-c]pyridin-7-yl)acetamide

Trifluoroacetic acid (1.48 g, 12.98 mmol, 1 mL) was added to the solution of tert-butyl 4-[5-[(2R,5S)-5-methyl-1-[2-oxo-2-[[1-(2-trimethylsilylethoxymethyl)pyrazolo[4,3-c]pyridin-7-yl]amino]acetyl]-2-piperidyl]-1,3-benzothiazol-2-yl]piperidine-1-carboxylate (500 mg, 681.21 μmol) in DCM (5 mL). Resulting solution was stirred at 20° C. for 15 hr. Then, it was subjected to HPLC (20-70% 0-5 min H₂O/MeOH/0.1% NH₄OH, flow: 30 ml/min; column: XBridge BEH C18 100×19 mm, 5 um), affording 2-oxo-N-(1H-pyrazolo[4,3-c]pyridin-7-yl)-2-[(2R,5S)-5-methyl-2-[2-(4-piperidyl)-1,3-benzothiazol-5-yl]-1-piperidyl]acetamide (220 mg, 436.84 μmol, 64.13% yield).

LCMS(ESI): [M]⁺ m/z: calcd 503.2; found 504.2; Rt=0.918 min.

### Step 3: Synthesis of 2-((2R,5S)-2-(2-(1-cyclopropylpiperidin-4-yl)benzo[d]thiazol-5-yl)-5-methylpiperidin-1-yl)-2-oxo-N-(1H-pyrazolo[4,3-c]pyridin-7-yl)acetamide (Compound 131)

(1-Ethoxycyclopropoxy)-trimethyl-silane (102.45 mg, 587.75 μmol, 118.17 μL) and 2-oxo-N-(1H-pyrazolo[4,3-c]pyridin-7-yl)-2-[(2R,5S)-5-methyl-2-[2-(4-piperidyl)-1,3-benzothiazol-5-yl]-1-piperidyl]acetamide (148 mg, 293.87 μmol) were dissolved in MeOH (3 mL). Acetic acid (35.30 mg, 587.75 μmol, 33.65 μL) was added thereto followed by sodium cyan borohydride (36.94 mg, 587.75 μmol). Resulting reaction mixture was stirred at 50° C. for 12 hr. Then, it was subjected to HPLC (40-40-80% 0-1-6 min H₂O/MeOH/0.1% NH₄OH, flow 30 ml/min; column: XBridge BEH C18 5 um 130 A), affording 2-oxo-N-(1H-pyrazolo[4,3-c]pyridin-7-yl)-2-[(2R,5S)-2-[2-(1-cyclopropyl-4-piperidyl)-1,3-benzothiazol-5-yl]-5-methyl-1-piperidyl]acetamide (112 mg, 206.00 μmol, 70.10% yield).

Compound 131: ¹H NMR (600 MHz, DMSO-d₆) δ (ppm) 0.30-0.41 (m, 4H), 0.73-1.22 (m, 4H), 1.34-2.34 (m, 10H), 2.88-3.13 (m, 4H), 3.77-4.11 (m, 2H), 5.58-5.75 (m, 1H), 7.38-7.43 (m, 1H), 7.90-8.08 (m, 2H), 8.30-8.54 (m, 2H), 8.89-8.96 (m, 1H), 11.14-11.24 (m, 1H), 13.10-13.17 (m, 1H).

LCMS(ESI): [M]⁺ m/z: calcd 543.2; found 544.2; Rt=2.211 min.

### Example 127. 2-((2R,5S)-2-(2-(1,4-dimethylpiperidin-4-yl)benzo[d]thiazol-5-yl)-5-methylpiperidin-1-yl)-2-oxo-N-(1H-pyrazolo[4,3-c]pyridin-7-yl)acetamide (Compound 132)

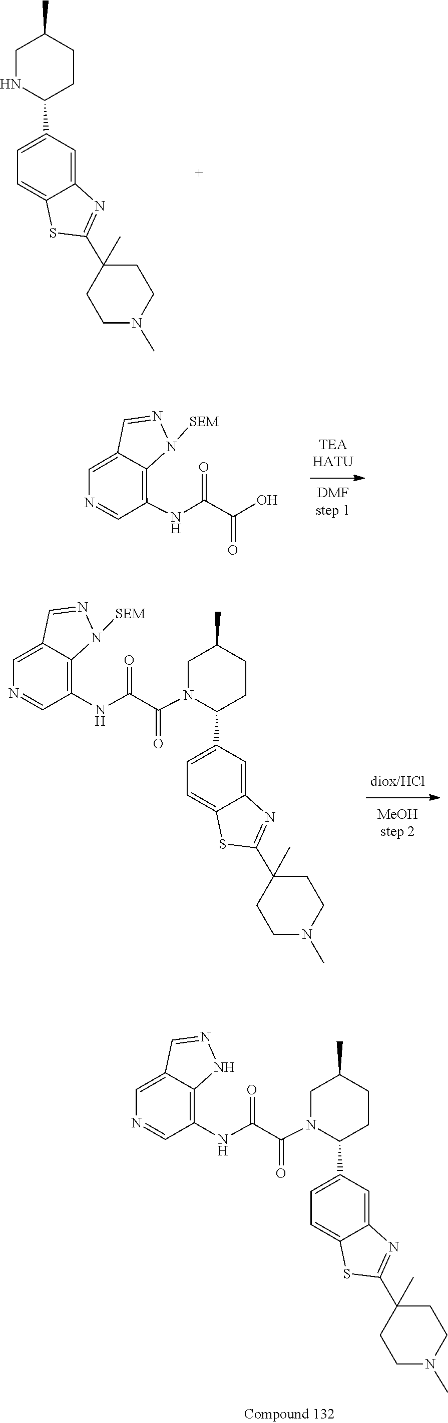 [SMILES: C[C@H]1CC[C@H](c2ccc3sc(C4(C)CCN(C)CC4)nc3c2)NC1 ; C[Si](C)(C)CCOCn1ncc2cncc(NC(=O)C(=O)O)c21 ; C[C@H]1CC[C@H](c2ccc3sc(C4(C)CCN(C)CC4)nc3c2)N(C(=O)C(=O)Nc2cncc3cnn(COCC[Si](C)(C)C)c23)C1 ; C[C@H]1CC[C@H](c2ccc3sc(C4(C)CCN(C)CC4)nc3c2)N(C(=O)C(=O)Nc2cncc3cn[nH]c23)C1]

Synthesis of 2-(1,4-dimethylpiperidin-4-yl)-5-((2R,5S)-5-methylpiperidin-2-yl)benzo[d]thiazole is given in by Intermediate 43.

### Step 1: Synthesis of 2-((2R,5S)-2-(2-(1,4-dimethylpiperidin-4-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A. Yield: 0.3 g of crude.

LCMS(ESI): [M]⁺ m/z: calcd 661.2; found 662.2; Rt=1.247 min.

### Step 2: Synthesis of 2-((2R,5S)-2-(2-(1,4-dimethylpiperidin-4-yl)benzo[d]thiazol-5-yl)-5-methylpiperidin-1-yl)-2-oxo-N-(1H-pyrazolo[4,3-c]pyridin-7-yl)acetamide (Compound 132)

To the stirred solution of 2-((2R,5S)-2-(2-(1,4-dimethylpiperidin-4-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide (0.3 g, 453.22 μmol) in MeOH (3 mL) hydrogen chloride solution 4.0M in dioxane (800.00 mg, 21.94 mmol, 1 mL) was added. The resulting mixture was stirred at 25° C. for 3 day. The reaction mixture was concentrated under reduce pressure and was then purified by reverse phase HPLC (SYSTEM 15-15-70% 0-1-6 min H₂O/MeOH/0.1% NH₄OH, flow 30 ml/min (loading pump 4 ml/min MeCN) target mass 531 column: Chromatorex 18 SMB 100-ST 100*19 mm 5 um) to give 2-((2R,5S)-2-(2-(1,4-dimethylpiperidin-4-yl)benzo[d]thiazol-5-yl)-5-methylpiperidin-1-yl)-2-oxo-N-(1H-pyrazolo[4,3-c]pyridin-7-yl)acetamide (57 mg, 107.21 μmol, 23.66% yield).

Compound 132: ¹H NMR (DMSO-d6, 600 MHz): δ (ppm) 1.05-1.08 (m, 3H), 1.33-1.42 (m, 4H), 1.75-1.96 (m, 5H), 2.08-2.37 (m, 9H), 2.90-2.92 (m, 1H), 3.79-4.12 (m, 2H), 5.59-5.75 (m, 1H), 7.40-7.44 (m, 1H), 7.91 (m, 1H), 8.03-8.10 (m, 1H), 8.23-8.52 (m, 2H), 8.90-8.96 (m, 1H), 11.10-11.19 (m, 1H), 13.05-13.11 (m, 1H).

LCMS(ESI): [M]⁺ m/z: calcd 531.2; found 532.2; Rt=1.835 min.

### Example 128. N-(imidazo[1,2-a]pyridin-7-yl)-2-((2R,5S)-5-methyl-2-(2-(2-(pyrrolidin-1-yl)propyl)benzo[d]thiazol-5-yl)piperidin-1-yl)-2-oxoacetamide (Compound 133)

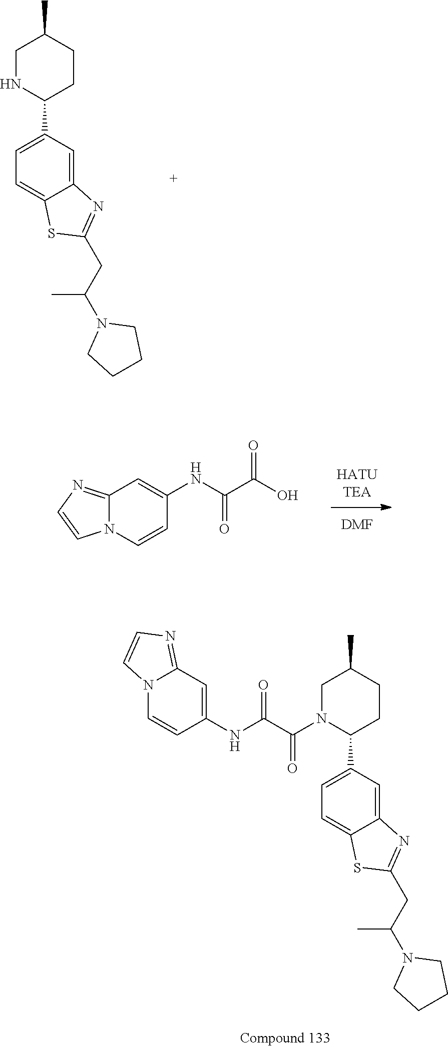

Prepared by general procedure scheme S2 step 6A. Yield: 133 mg (34.44%).

HPLC conditions: Column: XBridge C18 100*19 mm, 5 microM; 0-5 min 40-90% water-MeOH+0.1% NH₄OH; (loading pump 4 ml/min MeOH).

Compound 133: ¹H NMR (600 MHz, DMSO-d₆) δ (ppm) 1.05 (m, 6H), 1.37 (m, 1H), 1.71 (m, 5H), 2.02 (m, 3H), 2.31 (m, 1H), 2.61 (m, 4H), 3.00 (m, 3H), 3.76 (m, 1H), 5.50 (m, 1H), 7.06 (m, 1H), 7.37 (m, 1H), 7.47 (m, 1H), 7.97 (m, 4H), 8.46 (m, 1H), 11.14 (m, 1H).

LCMS(ESI): [M]⁺ m/z: calcd 530.2; found 531.2; Rt=1.937 min.

### Example 129. N-(4-amino-1H-pyrazolo[4,3-c]pyridin-7-yl)-2-(5-methyl-2-(6-methylpyridin-3-yl)piperidin-1-yl)-2-oxoacetamide (Compound 172, Compound 135)

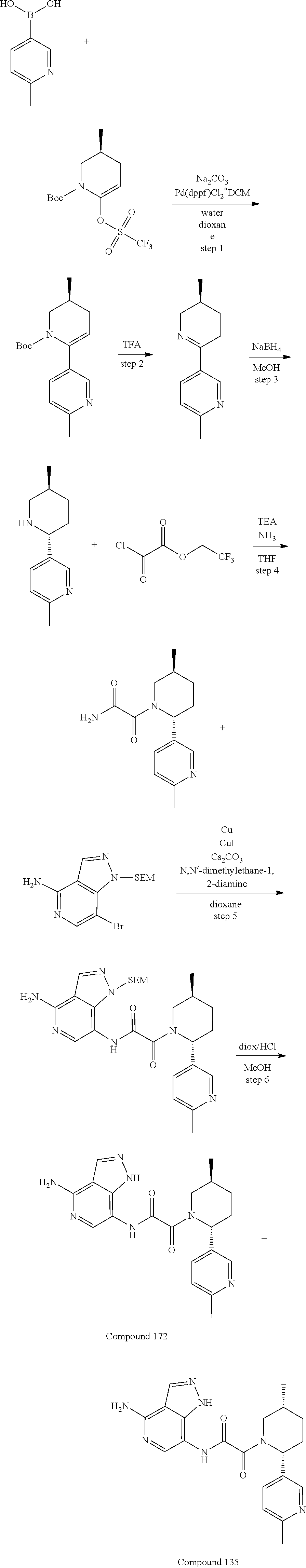

### Step 1: Synthesis of(S)-tert-butyl 5,6′-dimethyl-5,6-dihydro-[2,3′-bipyridine]-1 (4H)-carboxylate

Prepared by general procedure scheme S8 step 2. Yield: 2.5 g (50.11%).

CC conditions: The crude product was purified by silica gel with DCM/EtOAc, 0-100% as an eluent mixture.

LCMS(ESI): [M]⁺ m/z: calcd 288.2; found 289.2; Rt=1.079 min.

### Step 2: Synthesis of(S)-5,6′-dimethyl-3,4,5,6-tetrahydro-2,3′-bipyridine

Prepared by general procedure scheme S8 step 3. Yield: 1.6 g (98.03%).

LCMS(ESI): [M]⁺ m/z: calcd 188.2; found 189.2; Rt=0.526 min.

### Step 3: Synthesis of 2-methyl-5-((2R,5S)-5-methylpiperidin-2-yl)pyridine

Prepared by general procedure scheme S8 step 4. Yield: 1.5 g (92.76%).

LCMS(ESI): [M]⁺ m/z: calcd 190.2; found 191.2; Rt=0.259 min.

### Step 4: Synthesis of 2-((2R,5S)-5-methyl-2-(6-methylpyridin-3-yl)piperidin-1-yl)-2-oxoacetamide

To a solution of 2-methyl-5-[(2R,5S)-5-methyl-2-piperidyl]pyridine (1.6 g, 8.41 mmol) and TEA (1.70 g, 16.82 mmol, 2.34 mL) in THF (30 mL) was added 2,2,2-trifluoroethyl 2-chloro-2-oxo-acetate (2.40 g, 12.61 mmol) dropwise at 0° C. under argon. The reaction mixture was then stirred for 12 hr at rt. Then ammonia (143.20 mg, 8.41 mmol) was bubbled through for 10 min at 0° C. The reaction mixture was then stirred for 12 hr at rt. The reaction mixture was filtered off and the filtrate was evaporated in vacuum to give 2-oxo-2-[(2R,5S)-5-methyl-2-(6-methyl-3-pyridyl)-1-piperidyl]acetamide (1.5 g, 5.74 mmol, 68.27% yield).

LCMS(ESI): [M]⁺ m/z: calcd 261.2; found 262.2; Rt=0.643 min.

### Step 5: Synthesis of N-(4-amino-1-((2-(trimethylsilyl)ethoxy)methyl)-1H-pyrazolo[4,3-c]pyridin-7-yl)-2-((2R,5S)-5-methyl-2-(6-methylpyridin-3-yl)piperidin-1-yl)-2-oxoacetamide

Prepared by general procedure scheme S9 step 3A. Yield: 152 mg (26.22%).

HPLC conditions: Column: XBridge C18 100*19 mm, 5 microM; 0-5 min 40-90% water-MeOH+0.1% NH₄OH 30 ml/min; (loading pump 4 ml/min MeOH).

LCMS(ESI): [M]⁺ m/z: calcd 523.2; found 524.2; Rt=1.100 min.

### Step 6: Synthesis of N-(4-amino-1H-pyrazolo[4,3-c]pyridin-7-yl)-2-(5-methyl-2-(6-methylpyridin-3-yl)piperidin-1-yl)-2-oxoacetamide (Compound 172, Compound 135)

To a solution of N-[4-amino-1-(2-trimethylsilylethoxymethyl)pyrazolo[4,3-c]pyridin-7-yl]-2-oxo-2-[(2R,5S)-5-methyl-2-(6-methyl-3-pyridyl)-1-piperidyl]acetamide (152 mg, 290.24 μmol) in MeOH (3 mL) was added hydrogen chloride solution 4.0M in dioxane (1.59 g, 4.35 mmol, 1.50 mL, 10% purity) at 21° C. The resulting mixture was left to stir for 18 hr. The resulting mixture was evaporated to dryness. The residue was purified by RP-HPLC (column: Chromatorex 18 SMB100-5T 100×19 mm 5 um; 5-5-30% 0-1-5 min H₂O/MeCN/0.1% FA, flow: 30 ml/min) to give 83 mg mixture of cis/trans isomers. The isomers were separated by chiral HPLC (Column: CHIRALPAK AD-H (250×20 mm, 5 mkm)-II; Mobile Phase: Hexane:IPA:MeOH:NH₃, 60:20:20:0.2; Flow Rate: 12 ml/min) to give the two individual isomers Compound 135 N-(4-amino-1H-pyrazolo[4,3-c]pyridin-7-yl)-2-oxo-2-[(2R,5R)-5-methyl-2-(6-methyl-3-pyridyl)-1-piperidyl]acetamide (26 mg, 66.08 μmol) and Compound 172 N-(4-amino-1H-pyrazolo[4,3-c]pyridin-7-yl)-2-oxo-2-[(2R,5S)-5-methyl-2-(6-methyl-3-pyridyl)-1-piperidyl]acetamide (42 mg, 106.75 μmol, 36.78% yield).

Analytical: RT for Compound 135 (Chiralpak AD-H (250×4.6 mm, 5 mkm), Hexane (0.1% EDA):IPA:MeOH, 60:20:20)=26.67; RT for Compound 172 (Chiralpak AD-H (250×4.6 mm, 5 mkm), Hexane (0.1% EDA):IPA:MeOH, 60:20:20)=40.07.

Preparative: RT for Compound 135 (Chiralpak AD-H (250×20 mm, 5 mkm)-II; Hexane:IPA:MeOH:NH₃, 60:20:20:0.2)=26.33; RT for Compound 172 (Chiralcel OD-H (250*20 mm, 5 mkm), Hexane-IPA-MeOH, 70-15-15, 13 ml/min)=41.06.

Compound 172: ¹H NMR (600 MHZ, DMSO-d₆) δ (ppm) 0.91-1.04 (m, 3H), 1.30-1.41 (m, 1H), 1.58-1.77 (m, 1H), 1.83-1.97 (m, 1H), 2.00-2.29 (m, 2H), 2.41-2.46 (m, 3H), 2.71-3.27 (m, 1H), 3.40-4.11 (m, 1H), 4.99-5.89 (m, 1H), 6.59-7.04 (m, 2H), 7.17-7.75 (m, 3H), 8.08-8.22 (m, 1H), 8.26-8.53 (m, 1H), 9.57-10.54 (m, 1H), 12.62-13.35 (m, 1H).

LCMS(ESI): [M]⁺ m/z: calcd 393.2; found 394.2; Rt=0.984 min.

Compound 135: ¹H NMR (600 MHz, DMSO-d₆) δ (ppm) 0.58-0.84 (m, 3H), 1.02-1.13 (m, 1H), 1.28-2.33 (m, 4H), 2.42-2.47 (m, 3H), 2.53-2.60 (m, 1H), 3.55-4.29 (m, 1H), 5.03-5.77 (m, 1H), 6.58-6.94 (m, 2H), 6.96-7.61 (m, 2H), 7.62-7.75 (m, 1H), 8.09-8.46 (m, 2H), 9.54-10.61 (m, 1H), 12.61-13.42 (m, 1H).

LCMS(ESI): [M]⁺ m/z: calcd 393.2; found 394.2; Rt=1.008 min.

### Example 130. 2-((2R,5S)-5-methyl-2-(2-(2-(pyrrolidin-1-yl)propyl)benzo[d]thiazol-5-yl)piperidin-1-yl)-2-oxo-N-(1H-pyrazolo[4,3-c]pyridin-7-yl)acetamide (Compound 136)

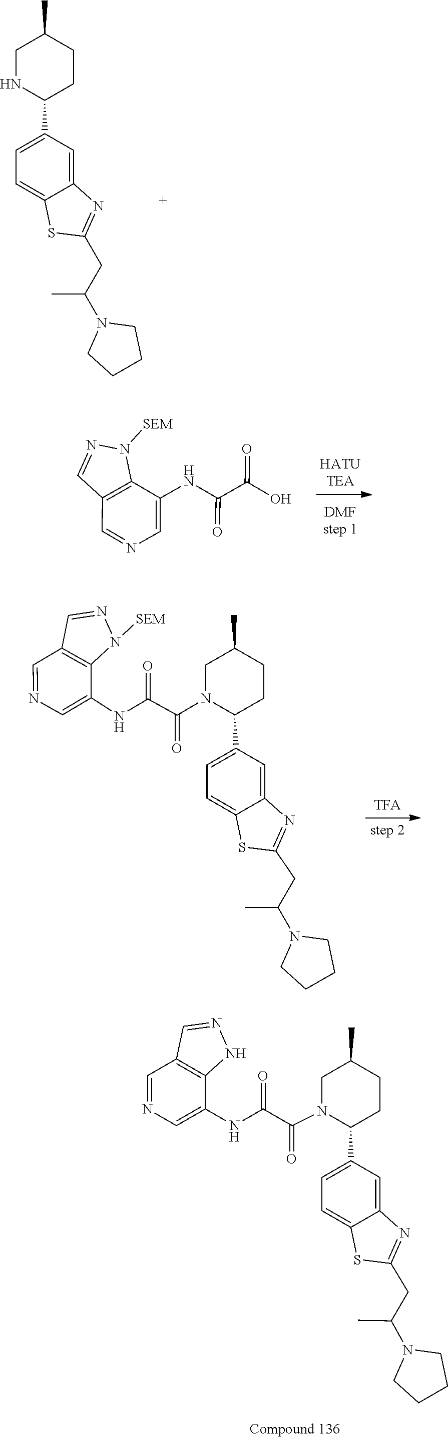 [SMILES: CC(Cc1nc2cc([C@H]3CC[C@H](C)CN3)ccc2s1)N1CCCC1 ; C[Si](C)(C)CCOCn1ncc2cncc(NC(=O)C(=O)O)c21 ; CC(Cc1nc2cc([C@H]3CC[C@H](C)CN3C(=O)C(=O)Nc3cncc4cnn(COCC[Si](C)(C)C)c34)ccc2s1)N1CCCC1 ; CC(Cc1nc2cc([C@H]3CC[C@H](C)CN3C(=O)C(=O)Nc3cncc4cn[nH]c34)ccc2s1)N1CCCC1]

### Step 1: Synthesis of 2-((2R,5S)-5-methyl-2-(2-(2-(pyrrolidin-1-yl)propyl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A. Yield: 133 mg (27.61%).

HPLC conditions: Column: XBridge C18 100*19 mm, 5 microM; 0-5 min 50-100% water-MeOH+0.1% NH₄OH, flow: 30 ml/min; (loading pump 4 ml/min MeOH).

LCMS(ESI): [M]⁺ m/z: calcd 661.2; found 662.2; Rt=1.093 min.

### Step 2: Synthesis of 2-((2R,5S)-5-methyl-2-(2-(2-(pyrrolidin-1-yl)propyl)benzo[d]thiazol-5-yl)piperidin-1-yl)-2-oxo-N-(1H-pyrazolo[4,3-c]pyridin-7-yl)acetamide (Compound 136)

A solution of 2-oxo-2-[(2R,5S)-5-methyl-2-[2-(2-pyrrolidin-1-ylpropyl)-1,3-benzothiazol-5-yl]-1-piperidyl]-N-[1-(2-trimethylsilylethoxymethyl)pyrazolo[4,3-c]pyridin-7-yl]acetamide (133 mg, 200.93 μmol) in TFA (4.44 g, 38.94 mmol, 3 mL) was stirred at 25° C. for 12 hr. The solvent was evaporated in vacuum and residue was purified by HPLC (Device (Mobile Phase, Column): SYSTEM 35-90% 0-5 min H₂O/MeOH/0.1% NH₄OH, flow: 30 ml/min (loading pump 4 ml/min MeOH) target mass 531.68 column: XBridge C18 100×19 mm, 5 um) to give 2-oxo-N-(1H-pyrazolo[4,3-c]pyridin-7-yl)-2-[(2R,5S)-5-methyl-2-[2-(2-pyrrolidin-1-ylpropyl)-1,3-benzothiazol-5-yl]-1-piperidyl]acetamide (70 mg, 131.66 μmol, 65.53% yield).

Compound 136: ¹H NMR (600 MHZ, DMSO-d₆) δ (ppm) 1.06 (m, 6H), 1.38 (m, 1H), 1.72 (m, 5H), 1.92 (m, 1H), 2.09 (m, 1H), 2.30 (m, 2H), 2.61 (m, 4H), 2.97 (m, 3H), 3.95 (m, 1H), 5.68 (m, 1H), 7.39 (m, 1H), 7.89 (m, 1H), 8.02 (m, 1H), 8.33 (m, 1H), 8.47 (m, 1H), 8.93 (m, 1H), 11.15 (m, 1H), 13.08 (m, 1H).

LCMS(ESI): [M]⁺ m/z: calcd 531.2; found 532.2; Rt=1.949 min.

### Example 131. N-(4-amino-1H-pyrazolo[4,3-c]pyridin-7-yl)-2-((2R,5S)-2-(4-cyanophenyl)-5-methylpiperidin-1-yl)-2-oxoacetamide (Compound 137)

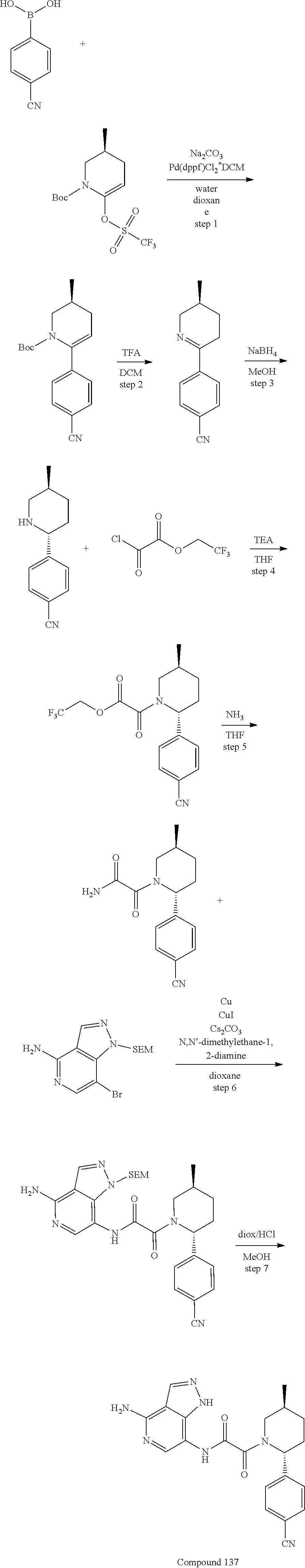

### Step 1: Synthesis of(S)-tert-butyl 6-(4-cyanophenyl)-3-methyl-3,4-dihydropyridine-1(2H)-carboxylate

Prepared by general procedure scheme S8 step 2. Yield: 1.9 g of crude.

LCMS(ESI): [M-Boc]⁺ m/z: calcd 198.2; found 199.2; Rt=4.039 min.

### Step 2: Synthesis of(S)-4-(5-methyl-3,4,5,6-tetrahydropyridin-2-yl)benzonitrile

Prepared by general procedure scheme S8 step 3. Yield: 0.35 g (27.72%).

¹H NMR (400 MHZ, DMSO-d₆) δ (ppm) 0.93 (d, 3H), 1.32 (m, 1H), 1.64 (m, 1H), 1.84 (m, 1H), 2.59 (m, 1H), 2.72 (m, 1H), 3.22 (m, 1H), 3.95 (m, 1H), 7.84 (m, 2H), 7.97 (m, 2H).

### Step 3: Synthesis of 4-((2R,5S)-5-methylpiperidin-2-yl)benzonitrile

Prepared by general procedure scheme S8 step 4. Yield: 0.33 g of crude.

LCMS(ESI): [M]⁺ m/z: calcd 200.2; found 201.2; Rt=0.776 min.

### Step 4: Synthesis of 2,2,2-trifluoroethyl 2-((2R,5S)-2-(4-cyanophenyl)-5-methylpiperidin-1-yl)-2-oxoacetate

Prepared by general procedure scheme S9 step 1. Yield: 0.61 g of crude.

LCMS(ESI): [M]⁺ m/z: calcd 354.2; found 355.2; Rt=1.170 min.

### Step 5: Synthesis of 2-((2R,5S)-2-(4-cyanophenyl)-5-methylpiperidin-1-yl)-2-oxoacetamide

Prepared by general procedure scheme S9 step 2. Yield: 0.2 g of crude.

LCMS(ESI): [M]⁺ m/z: calcd 271.2; found 272.2; Rt=0.912 min.

### Step 6: Synthesis of N-(4-amino-1-((2-(trimethylsilyl)ethoxy)methyl)-1H-pyrazolo[4,3-c]pyridin-7-yl)-2-((2R,5S)-2-(4-cyanophenyl)-5-methylpiperidin-1-yl)-2-oxoacetamide

Prepared by general procedure scheme S9 step 3A. Yield: 0.25 g of crude.

LCMS(ESI): [M]⁺ m/z: calcd 533.2; found 534.2; Rt=3.151 min.

### Step 7: Synthesis of N-(4-amino-1H-pyrazolo[4,3-c]pyridin-7-yl)-2-((2R,5S)-2-(4-cyanophenyl)-5-methylpiperidin-1-yl)-2-oxoacetamide (Compound 137)

Hydrogen chloride solution 4.0M in dioxane (1.71 g, 46.84 mmol, 2.13 mL) was added to a stirred solution of N-(4-amino-1-((2-(trimethylsilyl)ethoxy)methyl)-1H-pyrazolo[4,3-c]pyridin-7-yl)-2-((2R,5S)-2-(4-cyanophenyl)-5-methylpiperidin-1-yl)-2-oxoacetamide (0.25 g, 468.43 μmol) in MeOH (1.47 mL) and stirred at 20° C. for 8 hr. Then it was evaporated in vacuum, residue dissolved in MeOH (3 mL), quenched with TEA (0.2 g) and submitted to HPLC. 37 mg of Pure product with 20% of cis-isomer was obtained after 3-rd HPLC (column: YMC Triart C18 100×20 mm, 5 um; 0-40% 0-5 min H₂O/MeCN/0.1% NH₄OH, flow rate: 30 ml/min) and purified by chiral HPLC to give pure N-(4-amino-1H-pyrazolo[4,3-c]pyridin-7-yl)-2-((2R,5S)-2-(4-cyanophenyl)-5-methylpiperidin-1-yl)-2-oxoacetamide (28.8 mg, 71.39 μmol, 15.24% yield).

Compound 137: ¹H NMR (600 MHz, DMSO-d₆) δ (ppm) 0.91-1.06 (m, 3H), 1.27-1.66 (m, 2H), 1.69-2.14 (m, 2H), 2.16-2.30 (m, 1H), 2.69-3.26 (m, 1H), 3.44-4.16 (m, 1H), 5.05-5.63 (m, 1H), 6.47-7.61 (m, 5H), 7.67-7.93 (m, 2H), 8.10-8.23 (m, 1H), 9.57-10.54 (m, 1H), 12.62-13.43 (m, 1H).

LCMS(ESI): [M]⁺ m/z: calcd 403.2; found 404.2; Rt=2.407 min.

### Example 132. N-(4-amino-1H-pyrazolo[4,3-c]pyridin-7-yl)-2-((2R,5S)-5-methyl-2-(3-(trifluoromethyl)phenyl)piperidin-1-yl)-2-oxoacetamide (Compound 138)

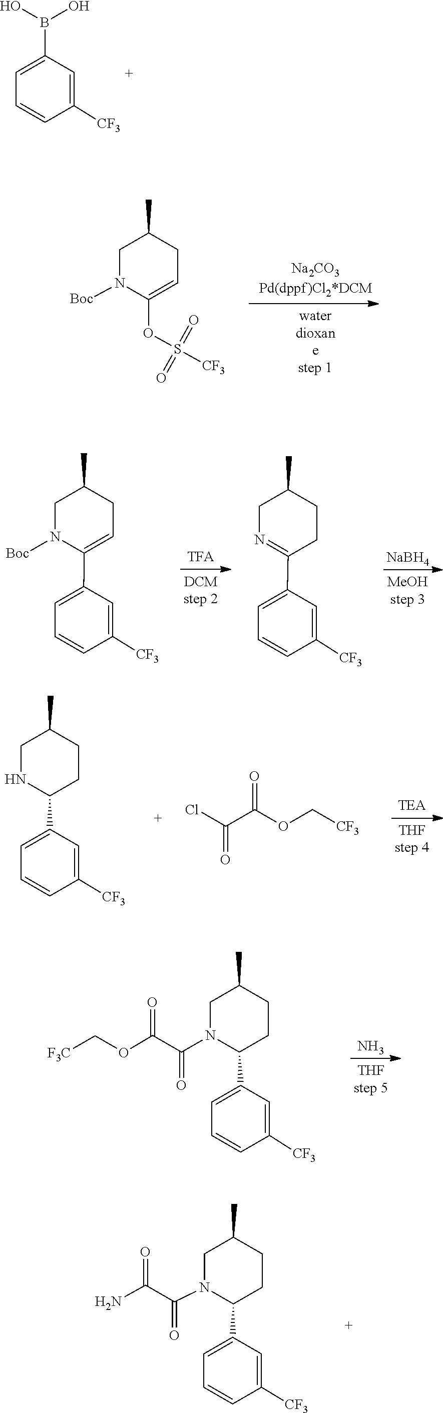

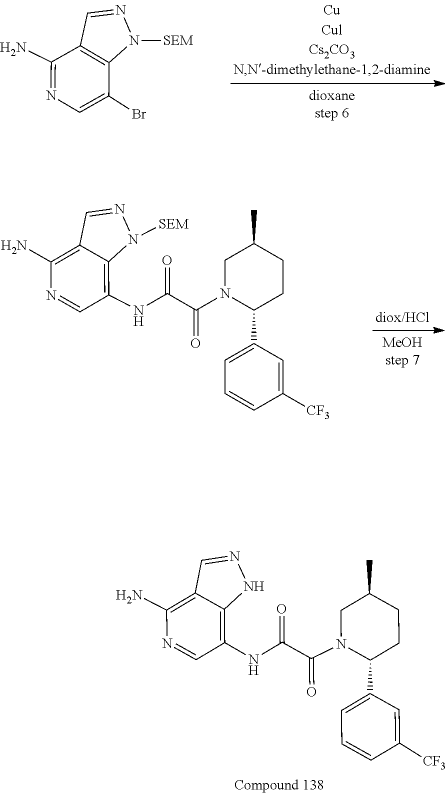 [SMILES: C[Si](C)(C)CCOCn1ncc2c(N)ncc(Br)c21 ; C[C@H]1CC[C@H](c2cccc(C(F)(F)F)c2)N(C(=O)C(=O)Nc2cnc(N)c3cnn(COCC[Si](C)(C)C)c23)C1 ; C[C@H]1CC[C@H](c2cccc(C(F)(F)F)c2)N(C(=O)C(=O)Nc2cnc(N)c3cn[nH]c23)C1]

### Step 1: Synthesis of(S)-tert-butyl 3-methyl-6-(3-(trifluoromethyl)phenyl)-3,4-dihydropyridine-1(2H)-carboxylate

Prepared by general procedure scheme S8 step 2. Yield: 2.7 g of crude.

LCMS(ESI): [M-Boc]⁺ m/z: calcd 241.2; found 242.2; Rt=4.863 min.

### Step 2: Synthesis of(S)-3-methyl-6-(3-(trifluoromethyl)phenyl)-2,3,4,5-tetrahydropyridine

Prepared by general procedure scheme S8 step 3. Yield: 0.48 g of crude.

LCMS(ESI): [M]⁺ m/z: calcd 241.2; found 242.2; Rt=0.706 min.

### Step 3: Synthesis of (2R,5S)-5-methyl-2-(3-(trifluoromethyl)phenyl)piperidine

Prepared by general procedure scheme S8 step 4. Yield: 0.45 g of crude.

LCMS(ESI): [M]⁺ m/z: calcd 243.2; found 244.2; Rt=0.900 min.

### Step 4: Synthesis of 2,2,2-trifluoroethyl 2-((2R,5S)-5-methyl-2-(3-(trifluoromethyl)phenyl)piperidin-1-yl)-2-oxoacetate

Prepared by general procedure scheme S9 step 1. Yield: 734 mg of crude.

LCMS(ESI): [M]⁺ m/z: calcd 397.2; found 398.2; Rt=1.530 min.

### Step 5: Synthesis of 2-((2R,5S)-5-methyl-2-(3-(trifluoromethyl)phenyl)piperidin-1-yl)-2-oxoacetamide

Prepared by general procedure scheme S9 step 2. Yield: 480 mg of crude.

LCMS(ESI): [M]⁺ m/z: calcd 314.2; found 315.2; Rt=1.198 min.

### Step 6: Synthesis of N-(4-amino-1-((2-(trimethylsilyl)ethoxy)methyl)-1H-pyrazolo[4,3-c]pyridin-7-yl)-2-((2R,5S)-5-methyl-2-(3-(trifluoromethyl)phenyl)piperidin-1-yl)-2-oxoacetamide

Prepared by general procedure scheme S9 step 3A. Yield: 0.44 g of crude.

LCMS(ESI): [M]⁺ m/z: calcd 576.2; found 577.2; Rt=3.940 min.

### Step 7: Synthesis of N-(4-amino-1H-pyrazolo[4,3-c]pyridin-7-yl)-2-((2R,5S)-5-methyl-2-(3-(trifluoromethyl)phenyl)piperidin-1-yl)-2-oxoacetamide (Compound 138)

Hydrogen chloride solution 4.0M in dioxane (2.09 g, 57.22 mmol, 2.61 mL) was added to a stirred solution of N-(4-amino-1-((2-(trimethylsilyl)ethoxy)methyl)-1H-pyrazolo[4,3-c]pyridin-7-yl)-2-((2R,5S)-5-methyl-2-(3-(trifluoromethyl)phenyl)piperidin-1-yl)-2-oxoacetamide (0.44 g, 762.98 μmol) in MeOH (3 mL) and stirred at 20° C. for 3 hr. Reaction mixture was concentrated and purified by HPLC (column: XBridge BEH C18 5 um 130 A; mobile phase: 20-55% 0-5 min H₂O/MeCN/0.1% NH₄OH, flow rate: 30 ml/min) to give crude product, 1-st fraction (87 mg, 62.9%) was purified (column: YMC Triart C18 100×20 mm, 5 um; mobile phase: 45-45-75% 0-1-5 min H₂O/MeOH/0.1% NH₄OH, flow rate: 30 ml/min) to give pure N-(4-amino-1H-pyrazolo[4,3-c]pyridin-7-yl)-2-((2R,5S)-5-methyl-2-(3-(trifluoromethyl)phenyl)piperidin-1-yl)-2-oxoacetamide (29 mg, 64.96 μmol, 8.51% yield).

Compound 138: ¹H NMR (500 MHz, DMSO-d₆) δ (ppm) 0.83-1.07 (m, 3H), 1.19-1.45 (m, 1H), 1.56-1.85 (m, 1H), 1.84-1.98 (m, 1H), 2.01-2.40 (m, 2H), 2.69-3.11 (m, 1H), 3.71-4.15 (m, 1H), 5.06-6.02 (m, 1H), 6.60-7.04 (m, 2H), 7.46-7.69 (m, 5H), 8.14-8.22 (m, 1H), 9.54-10.66 (m, 1H), 12.65-13.46 (m, 1H).

LCMS(ESI): [M]⁺ m/z: calcd 446.2; found 447.2; Rt=2.562 min.

### Example 133. N-(4-amino-1H-pyrazolo[4,3-c]pyridin-7-yl)-2-((2R,5S)-5-methyl-2-(rac-(R)-tetrahydro-2H-pyran-3-yl)piperidin-1-yl)-2-oxoacetamide (Compound 140)

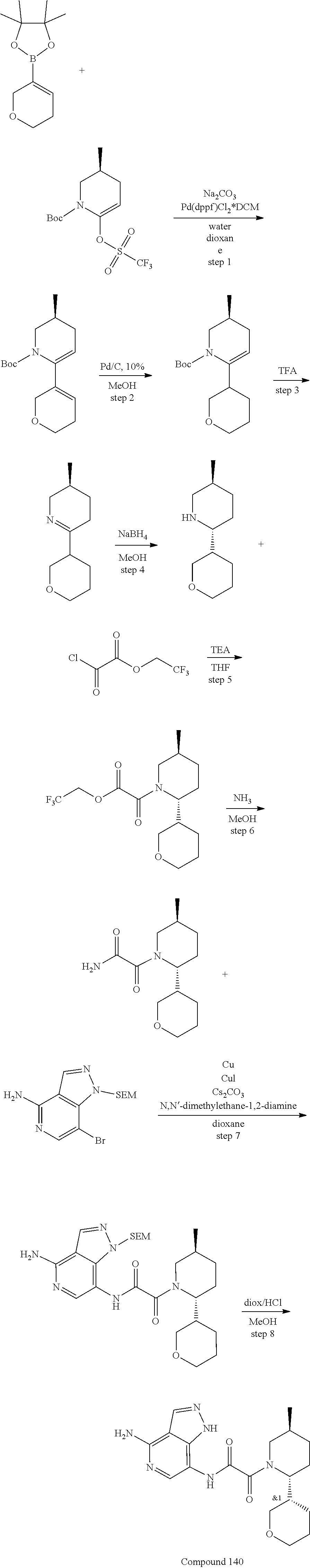

### Step 1: Synthesis of (S)-tert-butyl 6-(3,6-dihydro-2H-pyran-4-yl)-3-methyl-3,4-dihydropyridine-1(2H)-carboxylate

Prepared by general procedure scheme S8 step 2. Yield: 6 g of crude.

LCMS(ESI): [M-Boc]⁺ m/z: calcd 179.2; found 180.2; Rt=1.551 min.

### Step 2: Synthesis of (3S)-tert-butyl 3-methyl-6-(tetrahydro-2H-pyran-3-yl)-3,4-dihydropyridine-1(2H)-carboxylate

To a solution of tert-butyl (3S)-6-(3,6-dihydro-2H-pyran-5-yl)-3-methyl-3,4-dihydro-2H-pyridine-1-carboxylate (6.00 g, 21.48 mmol) in MeOH (50 mL) was added palladium, 10% on carbon, Type 487, dry (228.55 mg, 2.15 mmol). The reaction was put under an atmosphere of hydrogen (43.29 mg, 21.48 mmol) (1 bar) and stirred vigorously at rt. After 24 hr, the catalyst was removed by filtration, and the filtrate was concentrated in vacuum to afford tert-butyl (3S)-3-methyl-6-tetrahydropyran-3-yl-3,4-dihydro-2H-pyridine-1-carboxylate (7.9 g, crude) as a yellow oil, which was used for the next step without purification.

LCMS(ESI): [M-Boc]⁺ m/z: calcd 181.2; found 182.2; Rt=1.420 min.

### Step 3: Synthesis of (3S)-3-methyl-6-(tetrahydro-2H-pyran-3-yl)-2,3,4,5-tetrahydropyridine

Prepared by general procedure scheme S8 step 3. Yield: 5 g of crude.

LCMS(ESI): [M]⁺ m/z: calcd 181.2; found 182.2; Rt=0.627 min.

### Step 4: Synthesis of (2R,5S)-5-methyl-2-(rac-(R)-tetrahydro-2H-pyran-3-yl)piperidine

Prepared by general procedure scheme S8 step 4. Yield: 2.6 g of crude.

LCMS(ESI): [M]⁺ m/z: calcd 183.2; found 184.2; Rt=0.511 min.

### Step 5: Synthesis of 2,2,2-trifluoroethyl 2-((2R,5S)-5-methyl-2-(rac-(R)-tetrahydro-2H-pyran-3-yl)piperidin-1-yl)-2-oxoacetate

Prepared by general procedure scheme S9 step 1. Yield: 3 g of crude.

LCMS(ESI): [M]⁺ m/z: calcd 337.2; found 338.2; Rt=1.110 min.

### Step 6: Synthesis of 2-((2R,5S)-5-methyl-2-(rac-(R)-tetrahydro-2H-pyran-3-yl)piperidin-1-yl)-2-oxoacetamide

To a solution of 2,2,2-trifluoroethyl 2-oxo-2-[(2R,5S)-5-methyl-2-tetrahydropyran-3-yl-1-piperidyl]acetate (3 g, 8.89 mmol) in MeOH (50 mL) ammonia (155.05 mg, 9.10 mmol) was bubbled through for 10 min at 0° C. The reaction mixture was then stirred for 12 hr at rt. The reaction mixture was evaporated in vacuum, the residue was quenched with THF and filtered off. The filtrate was evaporated in vacuum to give 2-oxo-2-[(2R,5S)-5-methyl-2-tetrahydropyran-3-yl-1-piperidyl]acetamide (0.8 g, 3.15 mmol, 35.37% yield).

LCMS(ESI): [M]⁺ m/z: calcd 254.2; found 255.2; Rt=0.941 min.

### Step 7: Synthesis of N-(4-amino-1-((2-(trimethylsilyl)ethoxy)methyl)-1H-pyrazolo[4,3-c]pyridin-7-yl)-2-((2R,5S)-5-methyl-2-(rac-(R)-tetrahydro-2H-pyran-3-yl)piperidin-1-yl)-2-oxoacetamide

Prepared by general procedure scheme S9 step 3A. Yield: 81 mg (14.16%).

HPLC conditions: Column: XBridge BEH C18 100*19 mm, 5 microM; 0-1-6 min 45-90% water-MeOH 30 ml/min; (loading pump 4 ml/min MeOH).

LCMS(ESI): [M]⁺ m/z: calcd 516.2; found 517.2; Rt=2.535 min.

### Step 8: Synthesis of N-(4-amino-1H-pyrazolo[4,3-c]pyridin-7-yl)-2-((2R,5S)-5-methyl-2-(rac-(R)-tetrahydro-2H-pyran-3-yl)piperidin-1-yl)-2-oxoacetamide (Compound 140)

To a solution of N-[4-amino-1-(2-trimethylsilylethoxymethyl)pyrazolo[4,3-c]pyridin-7-yl]-2-oxo-2-[(2R,5S)-5-methyl-2-[rac-(3R)-tetrahydropyran-3-yl]-1-piperidyl]acetamide (81.00 mg, 156.76 μmol) in MeOH (998.34 μL) was added hydrogen chloride solution 4.0M in dioxane (857.33 mg, 2.35 mmol, 1.07 mL, 10% purity) at 21° C. The resulting mixture was left to stir for 18 hr. The resulting mixture was evaporated to dryness. The residue was purified by RP-HPLC (column: XBridge C18 100×19 mm, 5 um; 10-60% 0-5 min H₂O/MeOH/0.1% NH₄OH, flow: 30 ml/min) to give Compound 140 N-(4-amino-1H-pyrazolo[4,3-c]pyridin-7-yl)-2-oxo-2-[(2R,5S)-5-methyl-2-[rac-(3R)-tetrahydropyran-3-yl]-1-piperidyl]acetamide (21.7 mg, 56.15 μmol, 35.82% yield).

Compound 140: ¹H NMR (600 MHz, DMSO-d₆) δ (ppm) 0.80-1.01 (m, 3H), 1.10-1.21 (m, 1H), 1.22-1.34 (m, 1H), 1.37-1.53 (m, 2H), 1.53-1.66 (m, 2H), 1.66-1.75 (m, 1H), 1.78-2.02 (m, 2H), 2.07-2.17 (m, 1H), 2.59-3.09 (m, 2H), 3.12-3.23 (m, 1H), 3.46-3.64 (m, 1H), 3.66-3.75 (m, 1H), 3.78-3.89 (m, 1H), 3.99-4.37 (m, 1H), 6.62-6.79 (m, 2H), 7.40-7.77 (m, 1H), 8.10-8.25 (m, 1H), 9.41-10.42 (m, 1H), 12.61-13.35 (m, 1H).

LCMS(ESI): [M]⁺ m/z: calcd 386.2; found 387.2; Rt=1.715 min.

### Example 134. 2-((2R,5S)-5-methyl-2-(2-((R)-1-methylpyrrolidin-3-yl)-2H-indazol-6-yl)piperidin-1-yl)-2-oxo-N-(1H-pyrazolo[4,3-c]pyridin-7-yl)acetamide (Compound 141)

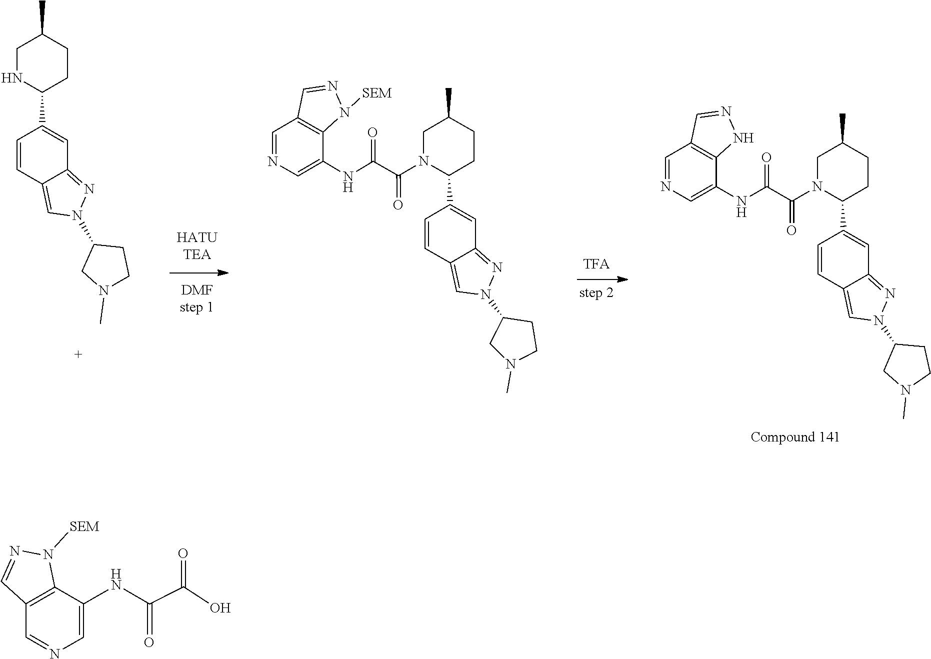 [SMILES: C[C@H]1CC[C@H](c2ccc3cn([C@@H]4CCN(C)C4)nc3c2)NC1 ; C[C@H]1CC[C@H](c2ccc3cn([C@@H]4CCN(C)C4)nc3c2)N(C(=O)C(=O)Nc2cncc3cnn(COCC[Si](C)(C)C)c23)C1 ; C[C@H]1CC[C@H](c2ccc3cn([C@@H]4CCN(C)C4)nc3c2)N(C(=O)C(=O)Nc2cncc3cn[nH]c23)C1 ; C[Si](C)(C)CCOCn1ncc2cncc(NC(=O)C(=O)O)c21]

The synthesis of 6-((2R,5S)-5-methylpiperidin-2-yl)-2-((R)-1-methylpyrrolidin-3-yl)-2H-indazole is prepared by general procedure scheme S7 step 3. Yield: 510 mg of crude.

LCMS(ESI): [M]⁺ m/z: calcd 298.2; found 299.2; Rt=0.622 min.

### Step 1: Synthesis of 2-((2R,5S)-5-methyl-2-(2-((R)-1-methylpyrrolidin-3-yl)-2H-indazol-6-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S7 step 5. Yield: 114.2 mg (26.31%).

HPLC conditions: Column: SunFire C18 100*19 mm, 5 microM; 0.6-6.5 min 10-60% MeCN+FA, flow: 30 ml/min; (loading pump 4 ml/min MeCN).

LCMS(ESI): [M]⁺ m/z: calcd 616.2; found 617.2; Rt=0.985 min.

### Step 2: Synthesis of 2-((2R,5S)-5-methyl-2-(2-((R)-1-methylpyrrolidin-3-yl)-2H-indazol-6-yl)piperidin-1-yl)-2-oxo-N-(1-((2-(trimethylsilyl)ethoxy)methyl)-1H-pyrazolo[4,3-c]pyridin-7-yl)acetamide (Compound 141)

2-Oxo-2-[(2R,5S)-5-methyl-2-[2-[(3R)-1-methylpyrrolidin-3-yl]indazol-6-yl]-1-piperidyl]-N-[1-(2-trimethylsilylethoxymethyl)pyrazolo[4,3-c]pyridin-7-yl]acetamide (114.2 mg, 185.14 μmol) was dissolved in TFA (1.5 mL) and the resulting mixture was stirred overnight. The reaction mixture was concentrated in vacuum and the residue was purified by HPLC (2-10 min 30-60% MeOH+NH₃ 30 ml/min) to obtain 2-oxo-N-(1H-pyrazolo[4,3-c]pyridin-7-yl)-2-[(2R,5S)-5-methyl-2-[2-[(3R)-1-methylpyrrolidin-3-yl]indazol-6-yl]-1-piperidyl]acetamide (44 mg, 90.43 μmol, 48.84% yield).

Compound 141: ¹H NMR (600 MHz, DMSO-d₆) δ (ppm) 0.78-1.08 (m, 3H), 1.28-1.45 (m, 1H), 1.67-1.84 (m, 1H), 1.86-2.02 (m, 1H), 2.05-2.24 (m, 2H), 2.26-2.32 (m, 4H), 2.39-2.45 (m, 1H), 2.54-2.58 (m, 1H), 2.79-2.87 (m, 2H), 2.88-3.22 (m, 2H), 3.70-4.17 (m, 1H), 5.13-5.23 (m, 1H), 5.49-5.88 (m, 1H), 6.95-7.11 (m, 1H), 7.55 (s, 1H), 7.63-7.74 (m, 1H), 8.18-8.42 (m, 2H), 8.42-8.57 (m, 1H), 8.86-9.00 (m, 1H), 10.95-11.31 (m, 1H), 12.84-13.27 (m, 1H).

LCMS(ESI): [M]⁺ m/z: calcd 486.2; found 487.2; Rt=1.372 min.

### Example 135. N-(4-amino-1H-pyrazolo[4,3-c]pyridin-7-yl)-2-((2R,5S)-5-methyl-2-(pyridin-3-yl)piperidin-1-yl)-2-oxoacetamide (Compound 144)

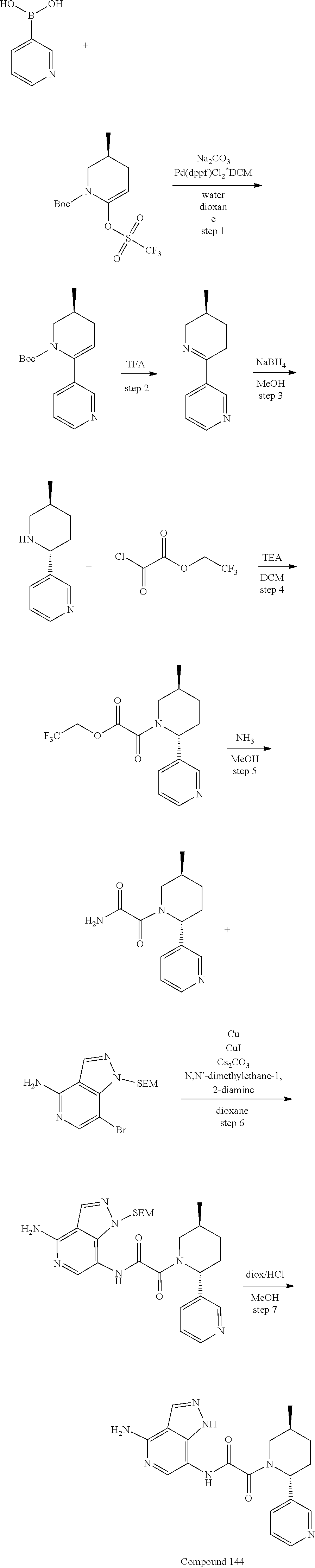

### Step 1: Synthesis of (S)-tert-butyl 5-methyl-5,6-dihydro-[2,3′-bipyridine]-1 (4H)-carboxylate

Prepared by general procedure scheme S8 step 2. Yield: 0.7 g of crude.

LCMS(ESI): [M]⁺ m/z: calcd 274.2; found 275.2; Rt=2.712 min.

### Step 2: Synthesis of (S)-5-methyl-3,4,5,6-tetrahydro-2,3′-bipyridine

Prepared by general procedure scheme S8 step 3. Yield: 0.4 g of crude.

LCMS(ESI): [M]⁺ m/z: calcd 174.2; found 175.2; Rt=0.998 min.

### Step 3: Synthesis of 3-((2R,5S)-5-methylpiperidin-2-yl)pyridine

Prepared by general procedure scheme S8 step 4. Yield: 0.4 g of crude.

LCMS(ESI): [M]⁺ m/z: calcd 176.2; found 177.2; Rt=0.455 min.

### Step 4: Synthesis of 2,2,2-trifluoroethyl 2-((2R,5S)-5-methyl-2-(pyridin-3-yl)piperidin-1-yl)-2-oxoacetate

Prepared by general procedure scheme S9 step 1. Yield: 0.74 g of crude.

LCMS(ESI): [M]⁺ m/z: calcd 330.2; found 331.2; Rt=2.203 min.

### Step 5: Synthesis of 2-((2R,5S)-5-methyl-2-(pyridin-3-yl)piperidin-1-yl)-2-oxoacetamide

Prepared by general procedure scheme S9 step 2. Yield: 202.3 mg (36.03%).

HPLC conditions: Column: XBridge C18 100*19 mm, 5 microM; 0.5-6.5 min 0-30% water-MeCN+NH₃ 30 ml/min; (loading pump 4 ml/min MeCN+NH₃).

LCMS(ESI): [M]⁺ m/z: calcd 247.2; found 248.2; Rt=0.631 min.

### Step 6: Synthesis of N-(4-amino-1-((2-(trimethylsilyl)ethoxy)methyl)-1H-pyrazolo[4,3-c]pyridin-7-yl)-2-((2R,5S)-5-methyl-2-(pyridin-3-yl)piperidin-1-yl)-2-oxoacetamide

Prepared by general procedure scheme S9 step 3A. Yield: 100 mg (40.43%).

HPLC conditions: Column: Chromatorex C18 100*19 mm, 5 microM; 0-1-5 min 10-10-35% water-MeCN+0.1% FA 30 ml/min; (loading pump 4 ml/min MeCN).

LCMS(ESI): [M]⁺ m/z: calcd 509.2; found 510.2; Rt=1.136 min.

### Step 7: Synthesis of N-(4-amino-1H-pyrazolo[4,3-c]pyridin-7-yl)-2-((2R,5S)-5-methyl-2-(pyridin-3-yl)piperidin-1-yl)-2-oxoacetamide (Compound 144)

Hydrogen chloride solution 4.0M in dioxane (536.52 mg, 14.72 mmol, 670.65 μL) was added to a stirred solution of N-[4-amino-1-(2-trimethylsilylethoxymethyl)pyrazolo[4,3-c]pyridin-7-yl]-2-oxo-2-[(2R,5S)-5-methyl-2-(3-pyridyl)-1-piperidyl]acetamide (0.1 g, 196.20 μmol) in MeOH (4.94 mL) and stirred at 20° C. for 8 hr. Volatiles was evaporated in vacuum, residue dissolved in MeOH (2 mL), neutralized with TEA (70 mg) and submitted to HPLC (column: YMC Triart C18 100×20 mm, 5 um; 0-30% 0-5 min H₂O/MeCN/0.1% NH₄OH, flow rate: 30 ml/min) to give pure N-(4-amino-1H-pyrazolo[4,3-c]pyridin-7-yl)-2-oxo-2-[(2R,5S)-5-methyl-2-(3-pyridyl)-1-piperidyl]acetamide (53 mg, 139.69 μmol, 71.20% yield).

Compound 144: ¹H NMR (600 MHZ, DMSO-d₆) δ (ppm) 0.88-1.08 (m, 3H), 1.23-1.45 (m, 1H), 1.60-1.79 (m, 1H), 1.85-1.97 (m, 1H), 2.04-2.30 (m, 2H), 2.63-3.27 (m, 1H), 3.43-4.16 (m, 1H), 5.04-6.04 (m, 1H), 6.57-6.98 (m, 2H), 6.99-7.44 (m, 1H), 7.48-7.81 (m, 2H), 8.14-8.59 (m, 3H), 9.60-10.56 (m, 1H), 12.64-13.34 (m, 1H).

LCMS(ESI): [M]⁺ m/z: calcd 379.2; found 380.2; Rt=1.257 min.

### Example 136. N-(4-amino-1H-pyrazolo[4,3-c]pyridin-7-yl)-2-oxo-2-[(2R,5S)-2-(2-methoxy-4-pyridyl)-5-methyl-1-piperidyl]acetamide (Compound 145)

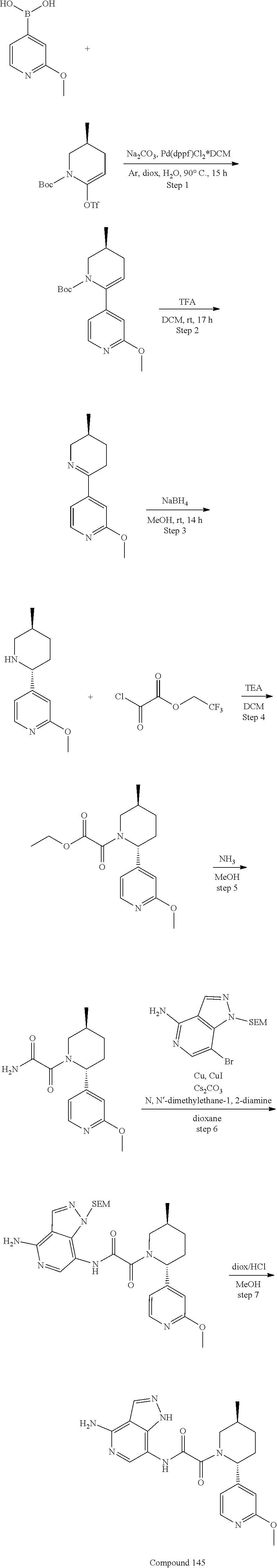

### Step 1: The Synthesis of tert-butyl (3S)-6-(2-methoxy-4-pyridyl)-3-methyl-3,4-dihydro-2H-pyridine-1-carboxylate

(2-methoxy-4-pyridyl) boronic acid (5 g, 32.69 mmol), Sodium carbonate (13.86 g, 130.77 mmol, 5.47 mL), tert-butyl (3S)-3-methyl-6-(trifluoromethylsulfonyloxy)-3,4-dihydro-2H-pyridine-1-carboxylate (11.29 g, 32.69 mmol) and Pd(dppf)Cl₂*DCM (1.33 g, 1.63 mmol) were mixed in H2O (20 mL) and dioxane (60 mL) under argon and stirred at 75° C. for 12 hr. Reaction mixture was diluted with water and desired product extracted with DCM, dried over Na2SO4 and concentrated in vacuo. tert-butyl (3S)-6-(2-methoxy-4-pyridyl)-3-methyl-3,4-dihydro-2H-pyridine-1-carboxylate (8 g, crude) was obtained.

LCMS(ESI): [M+H]⁺ m/z: calcd 304.2; found 305.2; Rt=1.519 min.

### Step 2: The Synthesis of 2-methoxy-4-[(3S)-3-methyl-2,3,4,5-tetrahydropyridin-6-yl]pyridine

To a solution of tert-butyl (3S)-6-(2-methoxy-4-pyridyl)-3-methyl-3,4-dihydro-2H-pyridine-1-carboxylate (8 g, 15.77 mmol) in DCM (40 mL) Trifluoroacetic acid (20 g, 175.41 mmol, 13.51 mL) was added and then stirred for 2 hr at 20° C. The reaction mixture was concentrated in vacuo, treated with aq. solution of NaHCO₃ and desired product was extracted with DCM (40 ml). Organic layer was dried over Na2SO4 and then concentrated in vacuo to give 2-methoxy-4-[(3S)-3-methyl-2,3,4,5-tetrahydropyridin-6-yl]pyridine (3.4 g, crude).

LCMS(ESI): [M+H]⁺ m/z: calcd 204.1; found 205.2; Rt=0.736 min.

### Step 3: The Synthesis of 2-methoxy-4-[(2S,5R)-5-methyl-2-piperidyl]

To a solution of 2-methoxy-4-[(3S)-3-methyl-2,3,4,5-tetrahydropyridin-6-yl]pyridine (3.00 g, 10.28 mmol) in Methanol (50.00 mL) Sodium Borohydride (583.41 mg, 15.42 mmol, 543.22 μL) was added and stirred for 12 hr at 25° C., then concentrated. The residue was treated with DCM and filtered off from insoluble impurities. Organic layer was concentrated in vacuo to give 2-methoxy-4-[(2S,5R)-5-methyl-2-piperidyl]pyridine (3 g, crude)

LCMS(ESI): [M+H]⁺ m/z: calcd 206.1; found 207.2; Rt=0.495 min.

### Step 4: The Synthesis of ethyl 2-oxo-2-[(2S,5R)-2-(2-methoxy-4-pyridyl)-5-methyl-1-piperidyl]acetate

2-methoxy-4-[(2S,5R)-5-methyl-2-piperidyl]pyridine (3 g, 7.27 mmol), and TEA (1.47 g, 14.54 mmol, 2.03 mL) was dissolved in DCM (40 mL), cooled with ice-water bath, then ethyl 2-chloro-2-oxo-acetate (1.39 g, 10.18 mmol, 1.14 mL) was added dropwise. Solution was stirred overnight at 25° C. Reaction mixture was washed with aqueous solution of NaHCO₃, dried over sodium sulphate and evaporated to give ethyl 2-oxo-2-[(2S,5R)-2-(2-methoxy-4-pyridyl)-5-methyl-1-piperidyl]acetate (3.5 g, crude)

LCMS(ESI): [M+H]⁺ m/z: calcd 306.1; found 307.2; Rt=1.345 min.

### Step 5: The Synthesis of 2-oxo-2-[(2R,5S)-2-(2-methoxy-4-pyridyl)-5-methyl-1-piperidyl]acetamide

Ethyl 2-oxo-2-[(2R,5S)-2-(2-methoxy-4-pyridyl)-5-methyl-1-piperidyl]acetate (3.5 g, 5.71 mmol) was dissolved in NH3/MeOH (20 mL) solution (10% of NH3 by weight) and stirred overnight. The RM was concentrated in vacuo, then dissolved in DCM (30 ml) and washed with water. Organic phase was dried over Na2SO4 and concentrated in vacuo, then subjected to column silica gel flash chromatography (gradient MTBE-Methanol) to give 2-oxo-2-[(2R,5S)-2-(2-methoxy-4-pyridyl)-5-methyl-1-piperidyl]acetamide (1 g, 3.61 mmol, 63.13% yield)

LCMS(ESI): [M+H]⁺ m/z: calcd 277.1; found 278.0; Rt=0.892 min.

### Step 6: The Synthesis of N-[4-amino-1-(2-trimethylsilylethoxymethyl)pyrazolo[4,3-c]pyridin-7-yl]-2-oxo-2-[(2R,5S)-5-methyl-2-(2-methylindazole-5-yl)-1-piperidyl]acetamide

Copper (2.87 mg, 45.24 μmol), Copper (I) iodide (86.15 mg, 452.36 μmol, 15.33 μL), cesium carbonate (589.55 mg, 1.81 mmol) was added to a stirred solution of 2-oxo-2-[(2R,5S)-2-(2-methoxy-4-pyridyl)-5-methyl-1-piperidyl]acetamide (250.89 mg, 904.72 μmol), 7-bromo-1-(2-trimethylsilylethoxymethyl)pyrazolo[4,3-c]pyridin-4-amine (326.11 mg, 949.95 μmol), (1R,2R)—N1,N2-dimethylcyclohexane-1,2-diamine (250.94 mg, 1.76 mmol) in 1,4-dioxane (7 mL) under Ar atmosphere and stirred at 110° C. for 48 hr in closed vial. Reaction mixture was filtered, solid washed with dioxane (2×3 mL), filtrate concentrated to give crude N-[4-amino-1-(2-trimethylsilylethoxymethyl)pyrazolo[4,3-c]pyridin-7-yl]-2-oxo-2-[(2R,5S)-2-(2-methoxy-4-pyridyl)-5-methyl-1-piperidyl]acetamide (0.48 g, 889.38 μmol, 98.31% yield).

LCMS(ESI): [M+H]⁺ m/z: calcd 539.3; found 540.4; Rt=3.051 min.

### Step 7: The Synthesis of N-(4-amino-1H-pyrazolo[4,3-c]pyridin-7-yl)-2-oxo-2-[(2R,5S)-2-(2-methoxy-4-pyridyl)-5-methyl-1-piperidyl]acetamide (Compound 145)

Hydrogen chloride solution 4.0M in dioxane (6.49 g, 177.88 mmol, 8.11 mL) was added to a stirred solution of N-[4-amino-1-(2-trimethylsilylethoxymethyl)pyrazolo[4,3-c]pyridin-7-yl]-2-oxo-2-[(2R,5S)-2-(2-methoxy-4-pyridyl)-5-methyl-1-piperidyl]acetamide (960.00 mg, 1.78 mmol) in Methanol (8 mL) and stirred at 20° C. for 18 hr. 18 hr was evaporated in vacuo, residue dissolved in methanol (8 mL), slowly quenched with TEA (0.5 g) and submitted to HPLC (column: Chromatorex 18 SMB100-5T 100×19 mm 5 um; 0-35% 0-5 min H2O/ACN/0.1% FA, flow rate: 30 ml/min) to give pure N-(4-amino-1H-pyrazolo[4,3-c]pyridin-7-yl)-2-oxo-2-[(2R,5S)-2-(2-methoxy-4-pyridyl)-5-methyl-1-piperidyl]acetamide (0.23 g, 504.98 μmol, 28.39% yield, HCOOH). 99+% ee by chiral HPLC (Column: Chiralcel OJ-H (250×4.6 mm, 5 mkm)-9, Mobile Phase: Hexane: IPA:MeOH, 50:25:25, Flow Rate: 0.6 ml/min).

[α]21D=+90.85° (C=0.1 g/100 mL EtOH).

¹H NMR (600 MHZ, dmso) δ 0.90-1.04 (m, 3H), 1.28-1.41 (m, 1H), 1.56-1.65 (m, 1H), 1.78-2.08 (m, 2H), 2.14-2.21 (m, 1H), 2.74-3.07 (m, 1H), 3.68-4.12 (m, 4H), 4.94-5.56 (m, 1H), 6.26-6.84 (m, 1H), 6.92-6.98 (m, 1H), 7.01-7.86 (m, 3H), 8.13-8.56 (m, 2H), 9.61-10.79 (m, 1H), 12.85-13.65 (m, 1H).

LCMS(ESI): [M+H]⁺ m/z: calcd 409.2; found 410.4; Rt=2.292 min.

### Example 137. 2-((2R,5S)-2-(benzo[d]thiazol-5-yl)-5-methylpiperidin-1-yl)-N-(4-(methylamino)-1H-pyrazolo[4,3-c]pyridin-7-yl)-2-oxoacetamide (Compound 146)

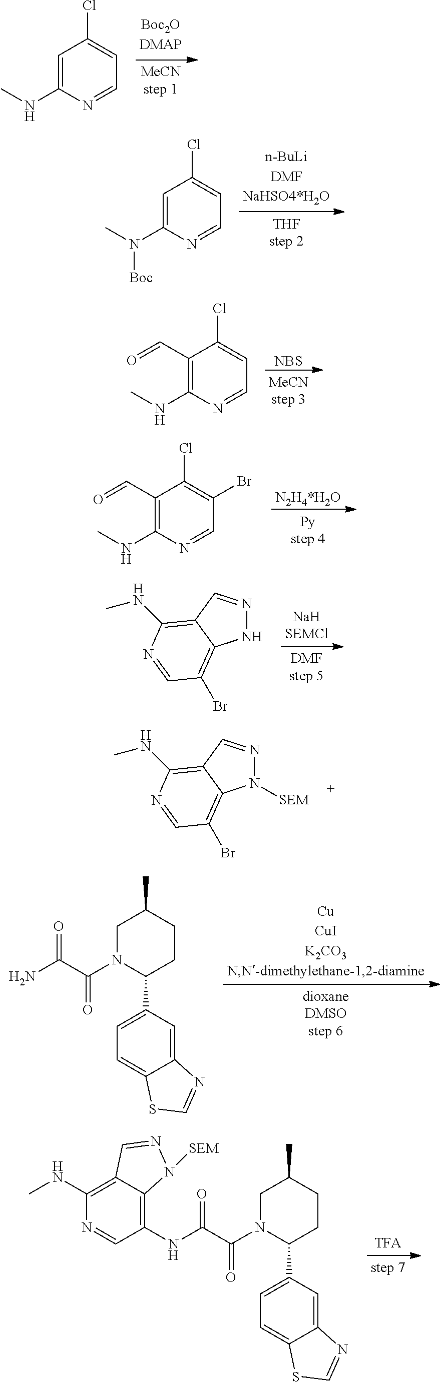

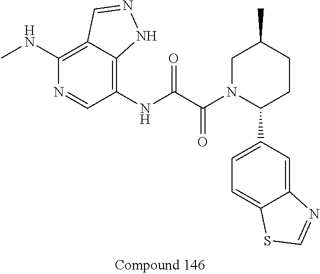 [SMILES: CNc1ncc(NC(=O)C(=O)N2C[C@@H](C)CC[C@@H]2c2ccc3scnc3c2)c2[nH]ncc12]

### Step 1: Synthesis of tert-butyl (4-chloropyridin-2-yl)(methyl)carbamate

Di-tert-butyl dicarbonate (7.96 g, 36.47 mmol, 8.37 mL) was added to the solution of 4-chloro-N-methyl-pyridin-2-amine (4 g, 28.05 mmol) and DMAP (342.72 mg, 2.81 mmol) in MeCN (60 mL). Resulting mixture was stirred at 60° C. for 16 hr. Then, water (20 ml) was added and stirring was continued for 20 min. Then, volatiles were removed under reduced pressure and residue was partitioned between 5% aq. NaHSO₄ solution (50 ml) and MTBE (70 ml). Organic layer was separated, dried over Na₂SO₄ and concentrated in vacuum, affording tert-butyl N-(4-chloro-2-pyridyl)-N-methyl-carbamate (6.8 g, 28.02 mmol, 99.87% yield).

LCMS(ESI): [M−t−Bu]⁺ m/z: calcd 186.2; found 187.2; Rt=1.585 min.

### Step 2: Synthesis of 4-chloro-2-(methylamino)nicotinaldehyde

n-Butyllithium, 23% (2.5M) in hexanes (8.58 g, 30.82 mmol, 12.39 mL, 23% purity) was added dropwise to the solution of tert-butyl N-(4-chloro-2-pyridyl)-N-methyl-carbamate (6.8 g, 28.02 mmol) in THF (100 mL) at −75° C. under argon. After addition was complete, resulting mixture was stirred at the same temperature for 40 min. Then, DMF (4.10 g, 56.04 mmol, 4.34 mL) was added dropwise keeping the temperature below −70° C. Then, cooling bath was removed and resulting mixture was slowly warmed to −40° C. At this point, solution of sodium hydrogen sulfate monohydrate (9.28 g, 67.24 mmol) in water (60 ml) was added in one portion and resulting mixture was stirred at ambient temperature overnight. After that, it was partitioned between MTBE (150 ml) and water (100 ml). Organic layer was washed with brine (100 ml), dried over Na₂SO₄ and concentrated under reduced pressure, leaving 4-chloro-2-(methylamino)pyridine-3-carbaldehyde (4.5 g, 26.38 mmol, 94.15% yield).

LCMS(ESI): [M]⁺ m/z: calcd 170.2; found 171.2; Rt=1.126 min.

### Step 3: Synthesis of 5-bromo-4-chloro-2-(methylamino)nicotinaldehyde

N-Bromosuccinimide (2.50 g, 14.07 mmol, 1.19 mL) was added to the solution of 4-chloro-2-(methylamino)pyridine-3-carbaldehyde (2 g, 11.72 mmol) in MeCN (50 mL). Resulting mixture was stirred at 50° C. for 16 hr. Then, solvent was removed under reduced pressure and residue was diluted with cold water (10 ml). Resulting yellow precipitate was filtered and dried, affording 5-bromo-4-chloro-2-(methylamino)pyridine-3-carbaldehyde (2.85 g, 11.42 mmol, 97.44% yield).

LCMS(ESI): [M]⁺ m/z: calcd 249.2; found 250.2; Rt=1.425 min.

### Step 4: Synthesis of 7-bromo-N-methyl-1H-pyrazolo[4,3-c]pyridin-4-amine

Hydrazine monohydrate (3.43 g, 68.54 mmol, 3.34 mL) was added to the solution of 5-bromo-4-chloro-2-(methylamino)pyridine-3-carbaldehyde (2.85 g, 11.42 mmol) in pyridine (30 mL). Resulting mixture was stirred at 115° C. for 20 hr. Then, volatiles were removed under reduced pressure and residue was diluted with water (40 ml). Resulting brown precipitate was filtered and dried, affording 7-bromo-N-methyl-1H-pyrazolo[4,3-c]pyridin-4-amine (2.42 g, 10.66 mmol, 93.30% yield).

LCMS(ESI): [M]⁺ m/z: calcd 227.2; found 228.2; Rt=0.561 min.

### Step 5: Synthesis of 7-bromo-N-methyl-1-((2-(trimethylsilyl)ethoxy)methyl)-1H-pyrazolo[4,3-c]pyridin-4-amine

Sodium hydride (in oil dispersion) 60% dispersion in mineral oil (532.85 mg, 13.32 mmol, 60% purity) was added portion wise to the cooled to 10° C. solution of 7-bromo-N-methyl-1H-pyrazolo[4,3-c]pyridin-4-amine (2.42 g, 10.66 mmol) in DMF (28.02 mL). After H₂ evolution ceased, 2-(chloromethoxy)ethyl-trimethyl-silane (1.87 g, 11.19 mmol, 1.98 mL) was added dropwise. Then, cooling bath was removed and resulting mixture was stirred at ambient temperature for 3 hr. After that, reaction was quenched with 120 ml of 10% aq. NH₄Cl and extracted with MTBE (2×100 ml). Combined organic layers were washed with water (100 ml) and brine (50 ml), dried over solid K₂CO₃ and concentrated under reduced pressure, leaving 7-bromo-N-methyl-1-(2-trimethylsilylethoxymethyl)pyrazolo[4,3-c]pyridin-4-amine (4 g, crude).

LCMS(ESI): [M]⁺ m/z: calcd 357.2; found 358.2; Rt=1.153 min.

### Step 6: Synthesis of 2-((2R,5S)-2-(benzo[d]thiazol-5-yl)-5-methylpiperidin-1-yl)-N-(4-(methylamino)-1-((2-(trimethylsilyl)ethoxy)methyl)-1H-pyrazolo[4,3-c]pyridin-7-yl)-2-oxoacetamide

Prepared by general procedure scheme S3 step 3A. Yield: 178 mg (37.53%).

HPLC conditions: Column: YMC Triart C18 100*20 mm, 5 microM; 0-1.5-6 min 60-60-90% water-MeOH+0.1% NH₄OH, flow: 30 ml/min; (loading pump 4 ml/min MeOH).

LCMS(ESI): [M]⁺ m/z: calcd 579.2; found 580.2; Rt=1.344 min.

### Step 7: Synthesis of 2-((2R,5S)-2-(benzo[d]thiazol-5-yl)-5-methylpiperidin-1-yl)-N-(4-(methylamino)-1H-pyrazolo[4,3-c]pyridin-7-yl)-2-oxoacetamide (Compound 146)

N-[4-(Methylamino)-1-(2-trimethylsilylethoxymethyl)pyrazolo[4,3-c]pyridin-7-yl]-2-oxo-2-[(2R,5S)-2-(1,3-benzothiazol-5-yl)-5-methyl-1-piperidyl]acetamide (178 mg, 307.01 μmol) was dissolved in TFA (2.22 g, 19.47 mmol, 1.5 mL). Resulting solution was stirred at 25° C. for 3 hr. Then, most of TFA was evaporated under reduced pressure and residue was subjected to HPLC (1-st run: 5-5-25% 0-1-5 min H₂O/MeCN/0.1% FA, flow: 30 ml/min; column: Chromatorex 18 SMB100-5T 100×19 mm 5 um. 2-nd run: 10-35% 0-5 min H₂O/MeCN/0.1% NH₄OH, flow: 30 ml/min; column: XBridge C18 100×19 mm, 5 um), affording N-[4-(methylamino)-1H-pyrazolo[4,3-c]pyridin-7-yl]-2-oxo-2-[(2R,5S)-2-(1,3-benzothiazol-5-yl)-5-methyl-1-piperidyl]acetamide (62 mg, 137.92 μmol, 44.92% yield).

Compound 146: ¹H NMR (600 MHZ, DMSO-d₆) δ (ppm) 0.72-1.15 (m, 3H), 1.23-1.51 (m, 1H), 1.66-1.83 (m, 1H), 1.84-1.99 (m, 1H), 2.05-2.31 (m, 1H), 2.32-2.46 (m, 1H), 2.54-2.84 (m, 1H), 2.86-3.01 (m, 3H), 3.49-4.18 (m, 1H), 5.11-5.92 (m, 1H), 7.22-7.38 (m, 1H), 7.42-7.58 (m, 1H), 7.59-7.81 (m, 1H), 7.88-8.07 (m, 1H), 8.07-8.26 (m, 2H), 9.29-9.46 (m, 1H), 9.60-10.65 (m, 1H), 12.52-13.50 (m, 1H).

LCMS(ESI): [M]⁺ m/z: calcd 449.2; found 450.2; Rt=1.215 min.

### Example 138. N-(4-amino-1H-pyrazolo[4,3-c]pyridin-7-yl)-2-(rac-(2S,5R)-2-cyclopentyl-5-methylpiperidin-1-yl)-2-oxoacetamide (Compound 147)

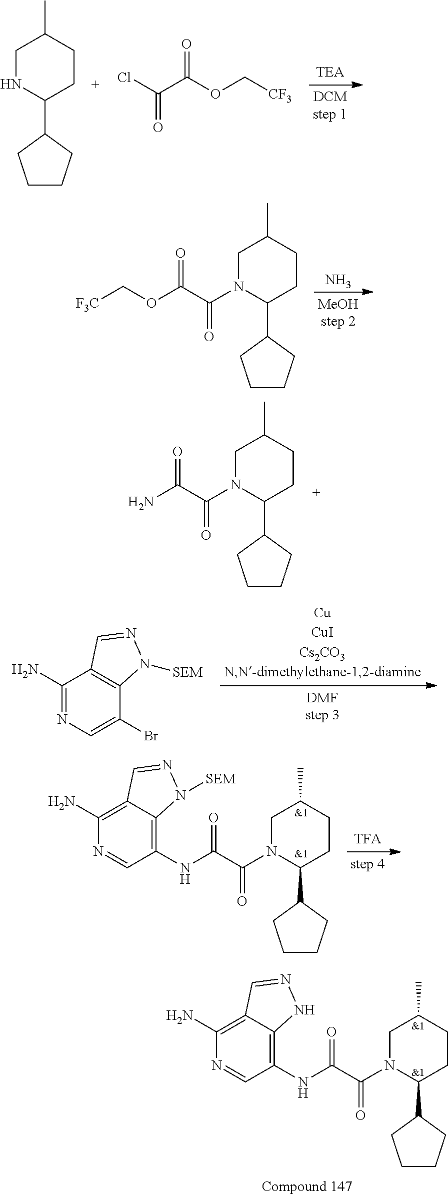

The synthesis of 2-cyclopentyl-5-methylpiperidine is given by the following procedure. Sodium borohydride (732.50 mg, 19.36 mmol, 684.58 μL) was added portion wise to a solution of 6-cyclopentyl-3-methyl-2,3,4,5-tetrahydropyridine (1.6 g, 9.68 mmol) in MeOH (20 mL). The mixture was stirred at rt for 12 hr. Water (50 ml) was added and resulting mixture was extracted with EtOAc (2×50 ml). Organic phase was dried over sodium sulfate, filtered and evaporated to obtain 2-cyclopentyl-5-methyl-piperidine (1.55 g, crude).

¹H NMR (400 MHZ, CDCl₃) δ (ppm) 0.78 (s, 3H), 1.03 (m, 4H), 1.62 (m, 11H), 2.16 (m, 2H), 2.96 (m, 1H).

GCMS: [M]⁺ m/z: calcd 167.4; found 168.2; Rt=6.657 min.

### Step 1: Synthesis of 2,2,2-trifluoroethyl 2-(2-cyclopentyl-5-methylpiperidin-1-yl)-2-oxoacetate

Prepared by general procedure scheme S9 step 1. Yield: 0.92 g of crude.

LCMS(ESI): [M]⁺ m/z: calcd 321.2; found 322.2; Rt=4.312 min.

### Step 2: Synthesis of 2-(2-cyclopentyl-5-methylpiperidin-1-yl)-2-oxoacetamide

Ammonia (1.22 g, 71.58 mmol) was bubbled through a solution of 2,2,2-trifluoroethyl 2-(2-cyclopentyl-5-methyl-1-piperidyl)-2-oxo-acetate (0.92 g, 2.86 mmol) in MeOH (25 mL) at rt. After stirring for 18 hr, the reaction mixture was evaporated to dryness to give 2-(2-cyclopentyl-5-methyl-1-piperidyl)-2-oxo-acetamide (0.6 g, 2.52 mmol, 87.93% yield) as a yellow gum.

LCMS(ESI): [M]⁺ m/z: calcd 238.2; found 239.2; Rt=3.060 min.

### Step 3: Synthesis of N-(4-amino-1-((2-(trimethylsilyl)ethoxy)methyl)-1H-pyrazolo[4,3-c]pyridin-7-yl)-2-(rac-(2S,5R)-2-cyclopentyl-5-methylpiperidin-1-yl)-2-oxoacetamide

Prepared by general procedure scheme S9 step 3A. Yield: 57 mg (13.57%).

HPLC conditions: Column: XBridge C18 100*19 mm, 5 microM; 0.5-6.5 min 40-70% water-MeCN+NH₃ 30 ml/min; (loading pump 4 ml/min MeCN).

LCMS(ESI): [M]⁺ m/z: calcd 500.2; found 501.2; Rt=1.518 min.

### Step 4: Synthesis of N-(4-amino-1H-pyrazolo[4,3-c]pyridin-7-yl)-2-(rac-(2S,5R)-2-cyclopentyl-5-methylpiperidin-1-yl)-2-oxoacetamide (Compound 147)

N-[4-Amino-1-(2-trimethylsilylethoxymethyl)pyrazolo[4,3-c]pyridin-7-yl]-2-oxo-2-[rac-(2S,5R)-2-cyclopentyl-5-methyl-1-piperidyl]acetamide (0.057 g, 113.84 μmol) was dissolved in TFA (389.41 mg, 3.42 mmol, 263.11 μL) and stirred at 20° C. for 3 hr. The reaction mixture was submitted to HPLC (2-10 min 30-65% MeOH+NH₃ 30/min; loading pump 4 ml/min MeOH+NH₃ column SunFire 19*100 mm). N-(4-Amino-1H-pyrazolo[4,3-c]pyridin-7-yl)-2-oxo-2-[rac-(2S,5R)-2-cyclopentyl-5-methyl-1-piperidyl]acetamide (0.005 g, 13.50 μmol, 11.86% yield) was obtained as a light-yellow solid.

Compound 147: ¹H NMR (600 MHZ, DMSO-d₆) δ (ppm) 0.96 (m, 3H), 1.16 (m, 2H), 1.30 (m, 2H), 1.52 (m, 6H), 1.73 (m, 1H), 1.92 (m, 2H), 2.96 (m, 1H), 3.39 (m, 1H), 3.69 (m, 1H), 4.18 (m, 1H), 6.67 (m, 2H), 7.72 (m, 1H), 8.13 (m, 1H), 10.29 (m, 1H), 12.64 (m, 1H).

LCMS(ESI): [M]⁺ m/z: calcd 370.2; found 371.2; Rt=2.121 min.

### Example 139. 2-oxo-N-(1H-pyrazolo[4,3-c]pyridin-7-yl)-2-[rac-(2R,5S)-5-methyl-2-(2-tetrahydrofuran-3-ylindazol-6-yl)-1-piperidyl]acetamide (Compound 148)

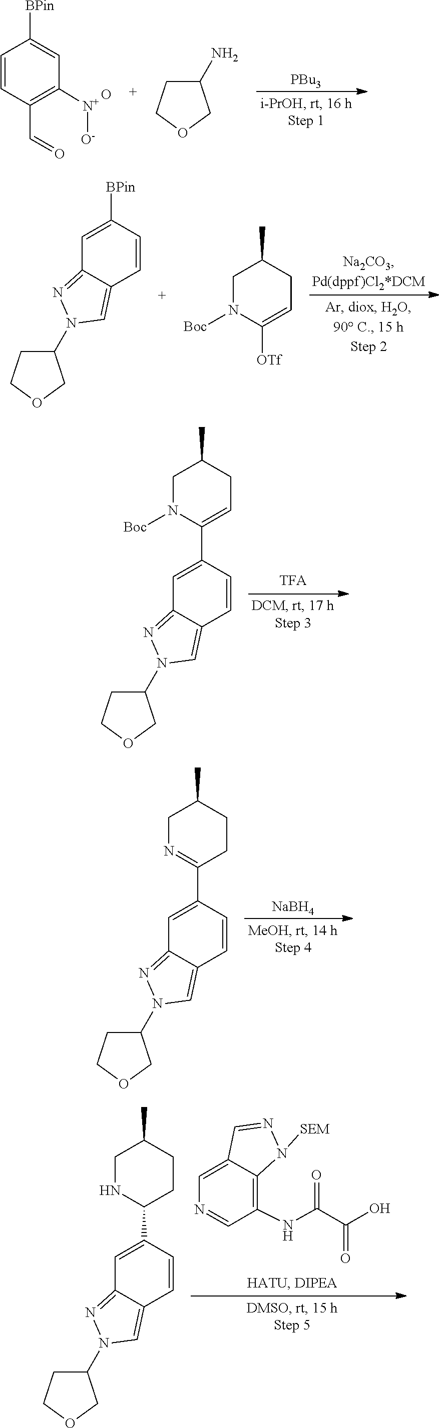

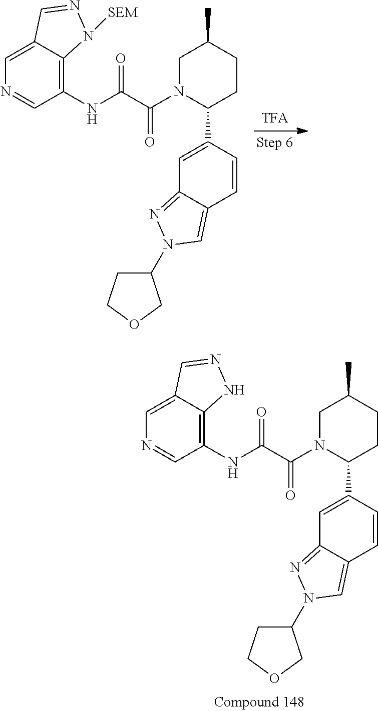 [SMILES: C[C@H]1CC[C@H](c2ccc3cn(C4CCOC4)nc3c2)N(C(=O)C(=O)Nc2cncc3cnn(COCC[Si](C)(C)C)c23)C1 ; C[C@H]1CC[C@H](c2ccc3cn(C4CCOC4)nc3c2)N(C(=O)C(=O)Nc2cncc3cn[nH]c23)C1]

### Step 1: The Synthesis of 2-tetrahydrofuran-3-yl-6-(4,4,5,5-tetramethyl-1,3,2-dioxaborolan-2-yl)indazole

### Prepared by General Procedure Scheme S7 Step 1

The crude product was purified by FCC (MTBE in HEX from 0% to 100%).

Yield: 1.1 g (38.8%)

LCMS(ESI): [M+H]⁺ m/z: calcd 314.2; found 315.0; Rt=1.376 min.

### Step 2: The Synthesis of tert-butyl rac-(3S)-3-methyl-6-(2-tetrahydrofuran-3-ylindazol-6-yl)-3,4-dihydro-2H-pyridine-1-carboxylate

Prepared by General Procedure Scheme S7 Step 2 Yield: 2.1 g (crude)

LCMS(ESI): [M+H]⁺ m/z: calcd 383.2; found 384.2; Rt=1.443 min.

### Step 3: The Synthesis of 6-[rac-(3S)-3-methyl-2,3,4,5-tetrahydropyridin-6-yl]-2-tetrahydrofuran-3-yl-indazole

### Prepared by General Procedure Scheme S7 Step 3

Yield: 0.7 g (45.11%)

LCMS(ESI): [M+H]⁺ m/z: calcd 283.2; found 284.2; Rt=0.645 min.

### Step 4: The Synthesis of 6-[rac-(2R,5S)-5-methyl-2-piperidyl]-2-tetrahydrofuran-3-yl-indazole

### Prepared by General Procedure Scheme S7 Step 4 (Method B)

Yield: 0.4 g (56.7%)

LCMS(ESI): [M+H]⁺ m/z: calcd 285.2; found 286.2; Rt=0.655 min.

### Step 5: The Synthesis of 2-oxo-2-[rac-(2R,5S)-5-methyl-2-(2-tetrahydrofuran-3-ylindazol-6-yl)-1-piperidyl]-N-[1-(2-trimethylsilylethoxymethyl)pyrazolo[4,3-c]pyridin-7-yl]acetamide

### Prepared by General Procedure Scheme S7 Step 5

HPLC conditions: 2-10 min 30-60% methnaol+FA, 30 ml/min (loading pump 4 ml methanol+FA) column: YMC-ACTUS TRIART C18 100*20 5 microM

Yield: 220 mg (42.3%)

LCMS(ESI): [M+H]⁺ m/z: calcd 603.4; found 604.4; Rt=1.337 min.

### Step 6: Synthesis of 2-oxo-N-(1H-pyrazolo[4,3-c]pyridin-7-yl)-2-[rac-(2R,5S)-5-methyl-2-(2-tetrahydrofuran-3-ylindazol-6-yl)-1-piperidyl]acetamide (Compound 148)

2-oxo-2-[rac-(2R,5S)-5-methyl-2-(2-tetrahydrofuran-3-ylindazol-6-yl)-1-piperidyl]-N-[1-(2-trimethylsilylethoxymethyl)pyrazolo[4,3-c]pyridin-7-yl]acetamide (0.22 g, 296.55 μmol, 3HCOOH) was dissolved in Trifluoroacetic acid, 99% (1.01 g, 8.90 mmol, 685.39 μL) and stirred at 20° C. for 3 hr. The reaction mixture was submitted to HPLC (2-10 min 0-80% methanol+NH₃ 30/min; loading pump 4 ml/min methanol+NH₃ column SunFire 19*100 mm). 2-oxo-N-(1H-pyrazolo[4,3-c]pyridin-7-yl)-2-[rac-(2R,5S)-5-methyl-2-(2-tetrahydrofuran-3-ylindazol-6-yl)-1-piperidyl]acetamide (0.027 g, 57.02 μmol, 19.23% yield) and 2-oxo-N-(1H-pyrazolo[4,3-c]pyridin-7-yl)-2-[rac-(2S,5S)-5-methyl-2-(2-tetrahydrofuran-3-ylindazol-6-yl)-1-piperidyl]acetamide (0.039 g, 82.36 μmol, 27.77% yield) was obtained as a light-yellow solids.

¹H NMR (600 MHz, dmso) δ 1.03-1.10 (m, 3H), 1.33-1.43 (m, 1H), 1.75-1.85 (m, 1H), 1.87-2.01 (m, 1H), 2.06-2.25 (m, 1H), 2.26-2.34 (m, 1H), 2.36-2.43 (m, 1H), 2.86-3.29 (m, 1H), 3.35-3.78 (m, 1H), 3.79-4.14 (m, 5H), 5.27-5.37 (m, 1H), 5.49-5.75 (m, 1H), 6.98-7.12 (m, 1H), 7.56 (s, 1H), 7.63-7.77 (m, 1H), 8.27-8.42 (m, 2H), 8.42-8.57 (m, 1H), 8.87-9.01 (m, 1H), 10.92-11.33 (m, 1H), 12.84-13.25 (m, 1H).

LCMS(ESI): [M+H]⁺ m/z: calcd 473.2; found 474.2; Rt=2.153 min.

### Example 140. N-(4-amino-1H-pyrazolo[4,3-c]pyridin-7-yl)-2-((2R,5S)-2-(3-((R)-2-(dimethylamino)propoxy)phenyl)-5-methylpiperidin-1-yl)-2-oxoacetamide (Compound 149)

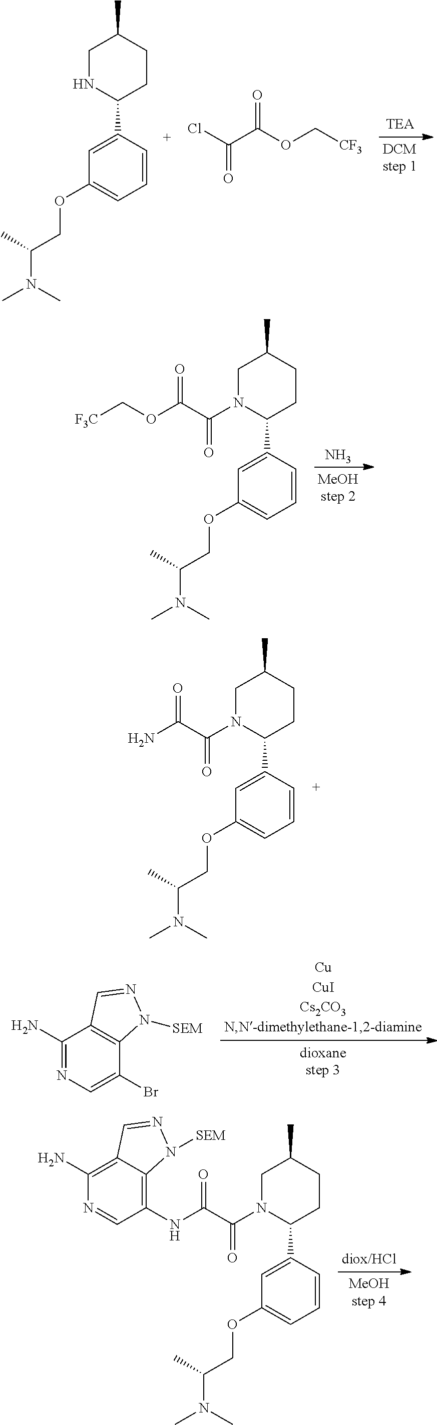

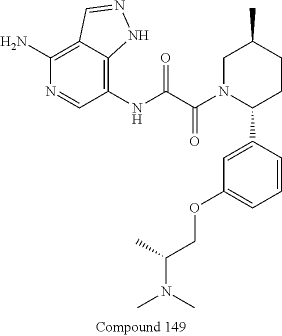 [SMILES: C[C@H]1CC[C@H](c2cccc(OC[C@@H](C)N(C)C)c2)N(C(=O)C(=O)Nc2cnc(N)c3cn[nH]c23)C1]

The synthesis of (R)—N,N-dimethyl-1-(3-((2R,5S)-5-methylpiperidin-2-yl)phenoxy)propan-2-amine is given by Intermediate 44.

### Step 1: Synthesis of 2,2,2-trifluoroethyl 2-((2R,5S)-2-(3-((R)-2-(dimethylamino)propoxy)phenyl)-5-methylpiperidin-1-yl)-2-oxoacetate

Prepared by general procedure scheme S9 step 1. Yield: 532 mg of crude.

LCMS(ESI): [M]⁺ m/z: calcd 430.2; found 431.2; Rt=0.906 min.

### Step 2: Synthesis of 2-((2R,5S)-2-(3-((R)-2-(dimethylamino)propoxy)phenyl)-5-methylpiperidin-1-yl)-2-oxoacetamide

2,2,2-Trifluoroethyl 2-oxo-2-[(2R,5S)-5-methyl-2-[3-[(2R)-2-(dimethylamino)propoxy]phenyl]-1-piperidyl]acetate (532 mg, 1.24 mmol) was dissolved in MeOH/NH₃ (5 mL) and the resulting mixture was stirred overnight. The reaction mixture was concentrated in vacuum to obtain 2-oxo-2-[(2R,5S)-5-methyl-2-[3-[(2R)-2-(dimethylamino)propoxy]phenyl]-1-piperidyl]acetamide (383 mg, crude).

LCMS(ESI): [M]⁺ m/z: calcd 347.2; found 348.2; Rt=0.666 min.

### Step 3: Synthesis of N-(4-amino-1-((2-(trimethylsilyl)ethoxy)methyl)-1H-pyrazolo[4,3-c]pyridin-7-yl)-2-((2R,5S)-2-(3-((R)-2-(dimethylamino)propoxy)phenyl)-5-methylpiperidin-1-yl)-2-oxoacetamide

Prepared by general procedure scheme S9 step 3A. Yield: 27.2 mg of crude.

HPLC conditions: Column: SunFire C18 100*19 mm, 5 microM; 2-10 min 0-45% MeCN+FA 30 ml/min; (loading pump 4 ml/min MeCN).

LCMS(ESI): [M]⁺ m/z: calcd 609.2; found 610.2; Rt=0.838 min.

### Step 4: Synthesis of N-(4-amino-1H-pyrazolo[4,3-c]pyridin-7-yl)-2-((2R,5S)-2-(3-((R)-2-(dimethylamino)propoxy)phenyl)-5-methylpiperidin-1-yl)-2-oxoacetamide (Compound 149)

A solution of N-[4-amino-1-(2-trimethylsilylethoxymethyl)pyrazolo[4,3-c]pyridin-7-yl]-2-oxo-2-[(2R,5S)-5-methyl-2-[3-[(2R)-2-(dimethylamino)propoxy]phenyl]-1-piperidyl]acetamide (27.2 mg, 44.60 μmol) in MeOH (1 mL) and dioxane/HCl (1 mL) was stirred at rt for 12 hr. Solvents were evaporated. Resulting crude product was purified by HPLC (0-80% MeOH+NH₃ 30 ml/min) to obtain N-(4-amino-1H-pyrazolo[4,3-c]pyridin-7-yl)-2-oxo-2-[(2R,5S)-5-methyl-2-[3-[(2R)-2-(dimethylamino)propoxy]phenyl]-1-piperidyl]acetamide (4.5 mg, 9.38 μmol, 21.04% yield).

Compound 149: ¹H NMR (600 MHZ, DMSO-d₆) δ (ppm)

LCMS(ESI): [M]⁺ m/z: calcd 479.2; found 480.2; Rt=1.822 min.

### Example 141. N-(4-amino-1H-pyrazolo[4,3-c]pyridin-7-yl)-2-((2R,5S)-5-methyl-2-(tetrahydro-2H-pyran-4-yl)piperidin-1-yl)-2-oxoacetamide (Compound 150)

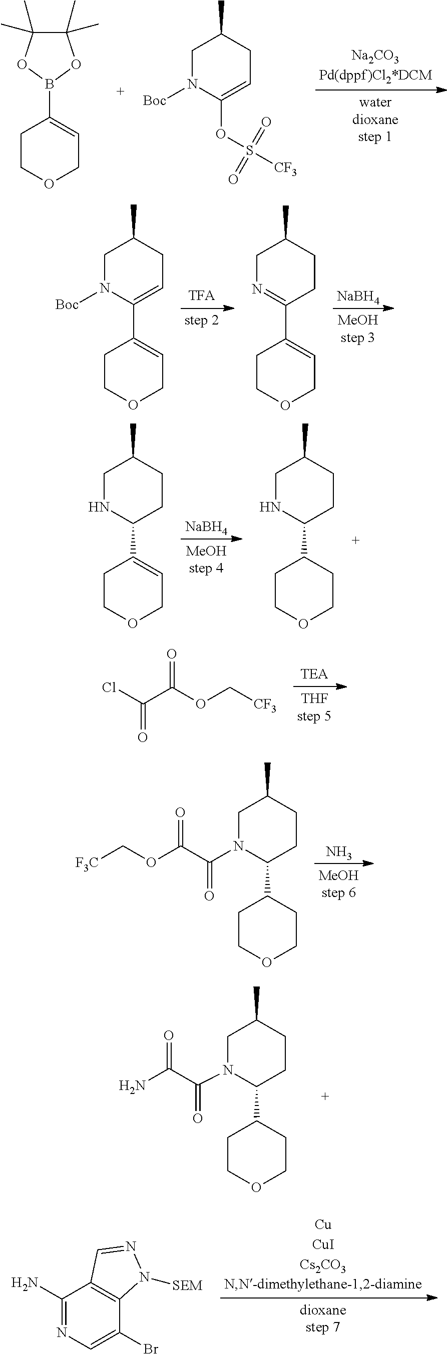

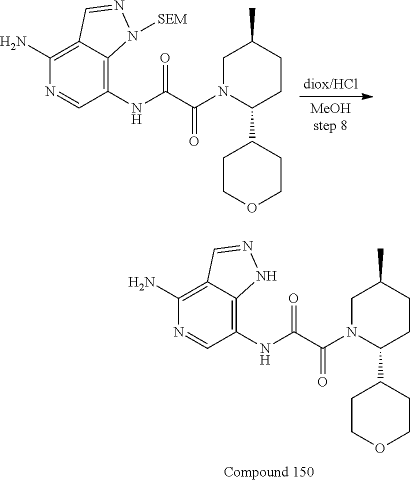

### Step 1: Synthesis of (S)-tert-butyl 6-(3,6-dihydro-2H-pyran-4-yl)-3-methyl-3,4-dihydropyridine-1(2H)-carboxylate

Prepared by general procedure scheme S8 step 2. Yield: 7.8 g of crude.

LCMS(ESI): [M-Boc]⁺ m/z: calcd 179.2; found 180.2; Rt=1.356 min.

### Step 2: Synthesis of (S)-6-(3,6-dihydro-2H-pyran-4-yl)-3-methyl-2,3,4,5-tetrahydropyridine

Prepared by general procedure scheme S8 step 3. Yield: 4.6 g of crude.

LCMS(ESI): [M]⁺ m/z: calcd 179.2; found 180.2; Rt=0.451 min.

### Step 3: Synthesis of (2R,5S)-2-(3,6-dihydro-2H-pyran-4-yl)-5-methylpiperidine

Prepared by general procedure scheme S8 step 4. Yield: 4 g of crude.

LCMS(ESI): [M]⁺ m/z: calcd 181.2; found 182.2; Rt=0.481 min.

### Step 4: Synthesis of (2R,5S)-5-methyl-2-(tetrahydro-2H-pyran-4-yl)piperidine

To a solution of (2R,5S)-2-(3,6-dihydro-2H-pyran-4-yl)-5-methyl-piperidine (4.00 g, 22.07 mmol) in MeOH (50 mL) was added palladium, 10% on carbon, Type 487, dry (234.83 mg, 2.21 mmol). The reaction was put under an atmosphere of hydrogen (44.48 mg, 22.07 mmol) (1 bar) and stirred vigorously at rt. After 24 hr, the catalyst was removed by filtration, and the filtrate was concentrated in vacuum to afford (2R,5S)-5-methyl-2-tetrahydropyran-4-yl-piperidine (4 g, 21.82 mmol, 98.90% yield) as a yellow oil, which was used for the next step without purification.

LCMS(ESI): [M]⁺ m/z: calcd 183.2; found 184.2; Rt=0.669 min.

### Step 5: Synthesis of 2,2,2-trifluoroethyl 2-((2R,5S)-5-methyl-2-(tetrahydro-2H-pyran-4-yl)piperidin-1-yl)-2-oxoacetate

Prepared by general procedure scheme S9 step 1. Yield: 2.5 g of crude.

LCMS(ESI): [M]⁺ m/z: calcd 337.2; found 338.2; Rt=0.964 min.

### Step 6: Synthesis of 2-((2R,5S)-5-methyl-2-(tetrahydro-2H-pyran-4-yl)piperidin-1-yl)-2-oxoacetamide

To a solution of 2,2,2-trifluoroethyl 2-oxo-2-[(2R,5S)-5-methyl-2-tetrahydropyran-4-yl-1-piperidyl]acetate (2.6 g, 7.71 mmol) in MeOH (50 mL) ammonia (155.05 mg, 9.10 mmol) was bubbled through for 10 min at 0° C. The reaction mixture was then stirred for 12 hr at rt. The reaction mixture was evaporated in vacuum; the residue was quenched with THF and filtered off. The filtrate was evaporated in vacuum and the residue was purified by silica gel flash chromatography eluting with a 0 to 100 percent MTBE-MeOH gradient to give 2-oxo-2-[(2R,5S)-5-methyl-2-tetrahydropyran-4-yl-1-piperidyl]acetamide (0.7 g, 2.75 mmol, 35.71% yield).

LCMS(ESI): [M]⁺ m/z: calcd 254.2; found 255.2; Rt=0.908 min.

### Step 7: Synthesis of N-(4-amino-1-((2-(trimethylsilyl)ethoxy)methyl)-1H-pyrazolo[4,3-c]pyridin-7-yl)-2-((2R,5S)-5-methyl-2-(tetrahydro-2H-pyran-4-yl)piperidin-1-yl)-2-oxoacetamide

Prepared by general procedure scheme S9 step 3A. Yield: 140 mg (34.45%).

HPLC conditions: Column: SunFire C18 100*19 mm, 5 microM; 0.5-6.5 min 20-45% MeCN+FA 30 ml/min; (loading pump 4 ml/min MeCN).

LCMS(ESI): [M]⁺ m/z: calcd 516.2; found 517.2; Rt=1.184 min.

### Step 8: Synthesis of N-(4-amino-1H-pyrazolo[4,3-c]pyridin-7-yl)-2-((2R,5S)-5-methyl-2-(tetrahydro-2H-pyran-4-yl)piperidin-1-yl)-2-oxoacetamide (Compound 150)

To a solution of N-[4-amino-1-(2-trimethylsilylethoxymethyl)pyrazolo[4,3-c]pyridin-7-yl]-2-oxo-2-[(2R,5S)-5-methyl-2-tetrahydropyran-4-yl-1-piperidyl]acetamide (140 mg, 270.95 μmol) in MeOH (2 mL) was added hydrogen chloride solution 4.0M in dioxane (1.48 g, 4.06 mmol, 1.41 mL, 10% purity) at 21° C. The resulting mixture was left to stir for 18 hr. The resulting mixture was evaporated to dryness. The residue was purified by RP-HPLC (column: Chromatorex 18 SNB100-5T 100*19 mm 5 um; 0-0-20% 0-1-6 min H₂O/MeCN/0.1% FA, flow: 30 ml/min, flow: 30 ml/min) to give Compound 150 N-(4-amino-1H-pyrazolo[4,3-c]pyridin-7-yl)-2-oxo-2-[(2R,5S)-5-methyl-2-tetrahydropyran-4-yl-1-piperidyl]acetamide (72.8 mg, 168.33 μmol, 62.13% yield, HCOOH).

Compound 150: ¹H NMR (600 MHZ, DMSO-d₆) δ (ppm) 0.77-1.01 (m, 3H), 1.03-1.22 (m, 2H), 1.24-1.57 (m, 3H), 1.57-1.74 (m, 2H), 1.76-2.19 (m, 3H), 2.65-3.07 (m, 1H), 3.32-3.34 (m, 2H), 3.45-4.23 (m, 4H), 6.89-7.50 (m, 2H), 7.73-7.85 (m, 1H), 8.15-8.38 (m, 1H), 9.34-10.54 (m, 1H), 12.80-13.59 (m, 1H).

LCMS(ESI): [M]⁺ m/z: calcd 386.2; found 387.2; Rt=1.760 min.

### Example 142. N-(4-amino-1H-pyrazolo[4,3-c]pyridin-7-yl)-2-((2R,5S)-2-(3,4-difluorophenyl)-5-methylpiperidin-1-yl)-2-oxoacetamide (Compound 151)

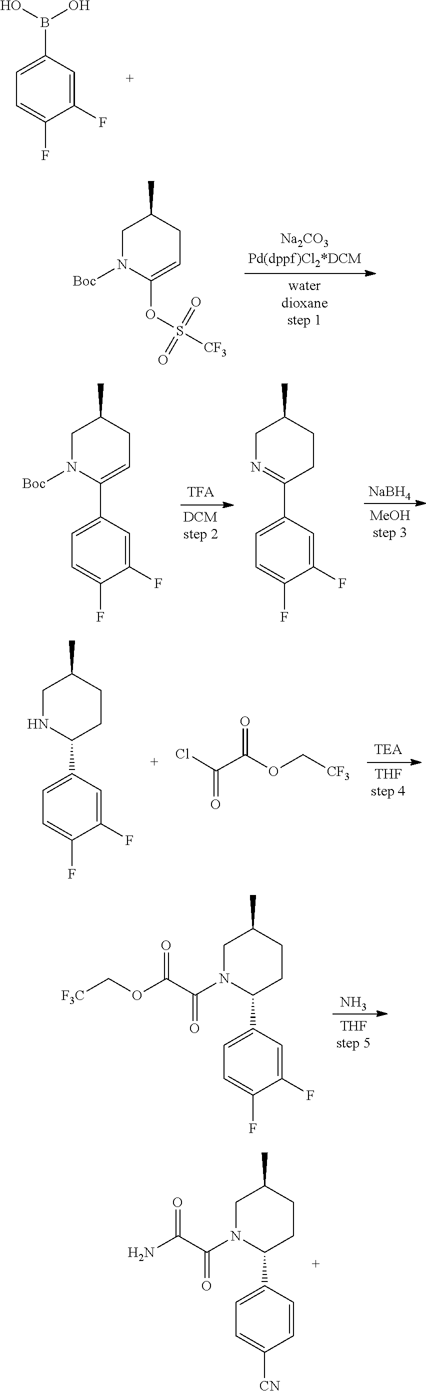

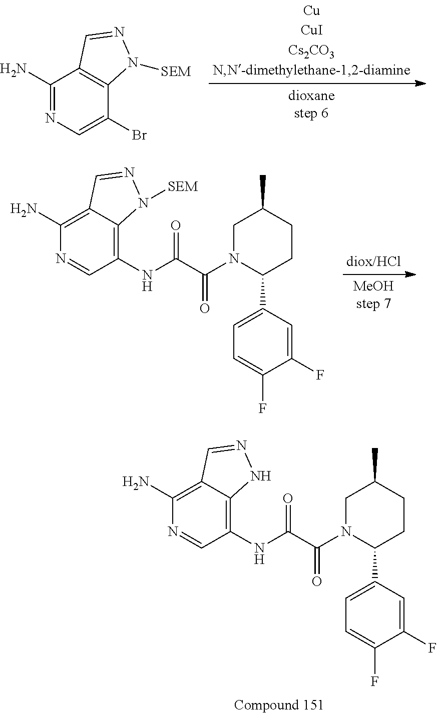

### Step 1: Synthesis of (S)-tert-butyl 6-(3,4-difluorophenyl)-3-methyl-3,4-dihydropyridine-1(2H)-carboxylate

Prepared by general procedure scheme S8 step 2. Yield: 6 g of crude.

¹H NMR (400 MHZ, CDCl₃) δ (ppm) 1.09 (d, 3H), 1.46 (s, 9H), 1.84 (m, 2H), 2.34 (m, 1H), 2.97 (m, 1H), 3.87 (m, 1H), 5.23 (m, 1H), 7.07 (m, 1H), 7.33 (m, 1H), 7.92 (m, 1H).

### Step 2: Synthesis of (S)-6-(3,4-difluorophenyl)-3-methyl-2,3,4,5-tetrahydropyridine

Prepared by general procedure scheme S8 step 3. Yield: 3 g of crude.

¹H NMR (400 MHZ, CDCl₃) δ (ppm) 1.02 (d, 3H), 1.52 (m, 1H), 1.68 (m, 1H), 1.94 (m, 1H), 2.40 (m, 1H), 2.94 (m, 1H), 3.29 (m, 1H), 4.02 (m, 1H), 7.16 (m, 1H), 7.52 (m, 1H), 7.66 (m, 1H).

### Step 3: Synthesis of (2R,5S)-2-(3,4-difluorophenyl)-5-methylpiperidine

Prepared by general procedure scheme S8 step 4. Yield: 2.2 g of crude.

¹H NMR (400 MHZ, CDCl₃) δ (ppm) 0.86 (d, 3H), 1.47 (m, 2H), 1.84 (m, 2H), 2.37 (m, 1H), 2.92 (m, 1H), 3.26 (m, 2H), 6.04 (m, 1H), 7.05 (m, 2H), 7.24 (m, 1H). Step 4: Synthesis of 2,2,2-trifluoroethyl 2-((2R,5S)-2-(3,4-difluorophenyl)-5-methylpiperidin-1-yl)-2-oxoacetate

Prepared by general procedure scheme S9 step 1. Yield: 1.35 g of crude.

LCMS(ESI): [M]⁺ m/z: calcd 365.2; found 366.2; Rt=3.929 min.

### Step 5: Synthesis of 2-((2R,5S)-2-(3,4-difluorophenyl)-5-methylpiperidin-1-yl)-2-oxoacetamide

Prepared by general procedure scheme S9 step 2. Yield: 0.8 g of crude.

LCMS(ESI): [M]⁺ m/z: calcd 282.2; found 283.2; Rt=3.028 min.

### Step 6: Synthesis of N-(4-amino-1-((2-(trimethylsilyl)ethoxy)methyl)-1H-pyrazolo[4,3-c]pyridin-7-yl)-2-((2R,5S)-2-(3,4-difluorophenyl)-5-methylpiperidin-1-yl)-2-oxoacetamide

Prepared by general procedure scheme S9 step 3A. Yield: 61.7 mg (10.66%).

HPLC conditions: Column: YMC Triart C18 100*20 mm, 5 microM; 0-5 min 65-80% water-MeOH+0.1% NH₄OH 30 ml/min; (loading pump 4 ml/min MeOH).

LCMS(ESI): [M]⁺ m/z: calcd 544.2; found 545.2; Rt=1.368 min.

### Step 7: Synthesis of N-(4-amino-1H-pyrazolo[4,3-c]pyridin-7-yl)-2-((2R,5S)-2-(3,4-difluorophenyl)-5-methylpiperidin-1-yl)-2-oxoacetamide (Compound 151)

Hydrogen chloride solution 4.0M in dioxane (2.40 g, 65.82 mmol, 3 mL) was added to a solution of N-[4-amino-1-(2-trimethylsilylethoxymethyl)pyrazolo[4,3-c]pyridin-7-yl]-2-oxo-2-[(2R,5S)-2-(3,4-difluorophenyl)-5-methyl-1-piperidyl]acetamide (61.70 mg, 113.28 μmol) in MeOH (3 mL). The reaction mixture was stirred at 20° C. for 48 hr, then evaporated was purified by RP-HPLC (column: Chromatex C18 5 um 130 A; 50-100% 0-1-6 min H₂O/MeOH, flow: 30 ml/min) to give 37 mg and was subjected to chiral HPLC purification (Column: Chiralpak IC (250-20 mm-5 m); Mobile phase:Hexane-IPA-MeOH, 50-25-25 Flow Rate: 12 mL/min) to give N-(4-amino-1H-pyrazolo[4,3-c]pyridin-7-yl)-2-((2R,5S)-2-(3,4-difluorophenyl)-5-methylpiperidin-1-yl)-2-oxoacetamide (0.05775 g, 124.60 μmol, 50.22% yield).

Compound 151: ¹H NMR (600 MHZ, DMSO-d₆) δ (ppm) 0.91-1.06 (m, 3H), 1.20-1.40 (m, 1H), 1.57-1.79 (m, 1H), 1.78-1.93 (m, 1H), 1.93-2.14 (m, 1H), 2.14-2.33 (m, 1H), 2.64-3.15 (m, 1H), 3.42-4.11 (m, 1H), 4.96-5.68 (m, 1H), 6.59-6.96 (m, 2H), 6.96-7.20 (m, 1H), 7.19-7.50 (m, 2H), 7.50-7.73 (m, 1H), 8.07-8.23 (m, 1H), 9.57-10.62 (m, 1H), 12.46-13.53 (m, 1H).

LCMS(ESI): [M]⁺ m/z: calcd 414.2; found 415.2; Rt=1.304 min.

### Example 143. N-(4-amino-1H-pyrazolo[4,3-c]pyridin-7-yl)-2-((2R,5S)-5-methyl-2-(1-methyl-1H-indazol-5-yl)piperidin-1-yl)-2-oxoacetamide (Compound 152)

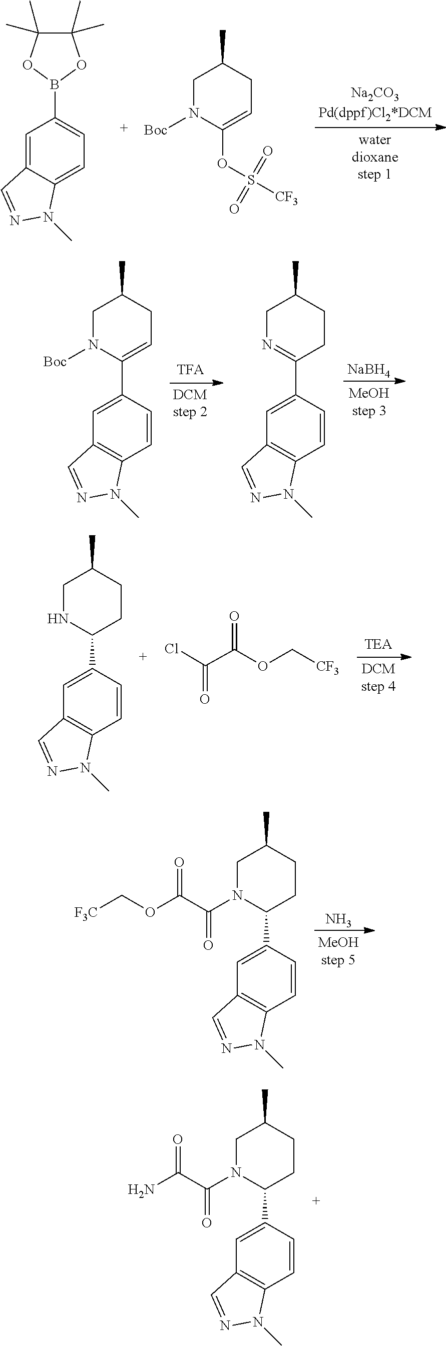

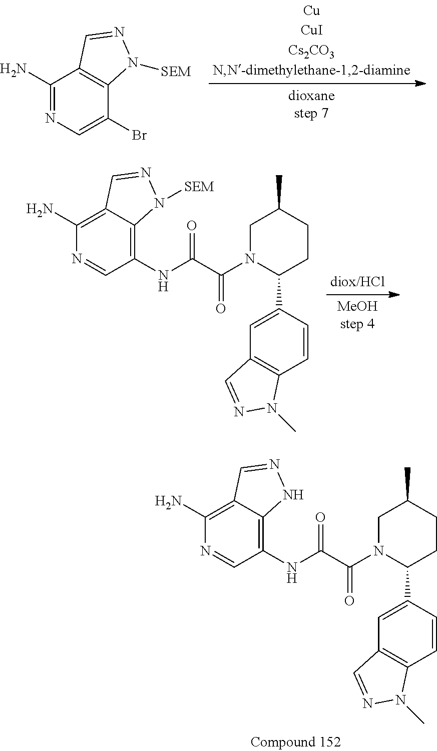

### Step 1: Synthesis of (S)-tert-butyl 3-methyl-6-(1-methyl-1H-indazol-5-yl)-3,4-dihydropyridine-1(2H)-carboxylate

Prepared by general procedure scheme S8 step 2. Yield: 2.5 g of crude.

LCMS(ESI): [M]⁺ m/z: calcd 327.2; found 328.2; Rt=1.306 min.

### Step 2: Synthesis of (S)-1-methyl-5-(5-methyl-3,4,5,6-tetrahydropyridin-2-yl)-1H-indazole

Prepared by general procedure scheme S8 step 3. Yield: 0.7 g of crude.

LCMS(ESI): [M]⁺ m/z: calcd 227.2; found 228.2; Rt=0.701 min.

### Step 3: Synthesis of 1-methyl-5-((2R,5S)-5-methylpiperidin-2-yl)-1H-indazole

Prepared by general procedure scheme S8 step 4. Yield: 0.5 g of crude.

LCMS(ESI): [M]⁺ m/z: calcd 229.2; found 230.2; Rt=0.642 min.

### Step 4: Synthesis of 2,2,2-trifluoroethyl 2-((2R,5S)-5-methyl-2-(1-methyl-1H-indazol-5-yl)piperidin-1-yl)-2-oxoacetate

Prepared by general procedure scheme S9 step 1. Yield: 0.8 g of crude.

LCMS(ESI): [M]⁺ m/z: calcd 383.2; found 384.2; Rt=1.401 min.

### Step 5: Synthesis of 2-((2R,5S)-5-methyl-2-(1-methyl-1H-indazol-5-yl)piperidin-1-yl)-2-oxoacetamide

2,2,2-Trifluoroethyl 2-oxo-2-[(2R,5S)-5-methyl-2-(1-methylindazol-5-yl)-1-piperidyl]acetate (0.8 g, 2.09 mmol) was dissolved in NH₃/MeOH. The resulting clear solution was left for 16 hr at 25° C. Upon completion of the reaction the resulting mixture was concentrated to dryness to obtain 2-oxo-2-[(2R,5S)-5-methyl-2-(1-methylindazol-5-yl)-1-piperidyl]acetamide (0.6 g, 2.00 mmol, 95.73% yield).

LCMS(ESI): [M]⁺ m/z: calcd 300.2; found 301.2; Rt=0.890 min.

### Step 6: Synthesis of N-(4-amino-1-((2-(trimethylsilyl)ethoxy)methyl)-1H-pyrazolo[4,3-c]pyridin-7-yl)-2-((2R,5S)-5-methyl-2-(1-methyl-1H-indazol-5-yl)piperidin-1-yl)-2-oxoacetamide

Prepared by general procedure scheme S9 step 3A. Yield: 220 mg (39.14%).

HPLC conditions: Column: XBridge BEH C18 100*19 mm, 5 microM; 0-1-6 min 40-40-65% water-MeCN+0.1% NH₄OH 30 ml/min; (loading pump 4 ml/min MeCN).

LCMS(ESI): [M]⁺ m/z: calcd 562.2; found 563.2; Rt=1.055 min.

### Step 7: Synthesis of N-(4-amino-1H-pyrazolo[4,3-c]pyridin-7-yl)-2-((2R,5S)-5-methyl-2-(1-methyl-1H-indazol-5-yl)piperidin-1-yl)-2-oxoacetamide (Compound 152)

Hydrogen chloride solution 4.0M in dioxane (3.15 g, 12.01 mmol, 3.00 mL, 13.9% purity) was added to a stirred solution of N-[4-amino-1-(2-trimethylsilylethoxymethyl)pyrazolo[4,3-c]pyridin-7-yl]-2-oxo-2-[(2R,5S)-5-methyl-2-(1-methylindazol-5-yl)-1-piperidyl]acetamide (220 mg, 390.95 μmol) in MeOH (3 mL) at 25° C. The resulting solution was stirred at 25° C. for 15 hr, then concentrated to dryness in vacuum and the residue was submitted to reverse phase HPLC (column: YMC Triart C18 100×20 mm, 5 um; mobile phase: 0-70% 0-5 min H₂O/MeOH, flow: 30 ml/min (loading pump 4 ml/min MeOH)) to afford Compound 152 N-(4-amino-1H-pyrazolo[4,3-c]pyridin-7-yl)-2-oxo-2-[(2R,5S)-5-methyl-2-(1-methylindazol-5-yl)-1-piperidyl]acetamide (24.4 mg, 48.28 μmol, 12.35% yield, 2HCl) as light-yellow solid (1st fraction after HPLC)

Compound 152: ¹H NMR (500 MHZ, DMSO-d₆) δ (ppm) 1.03-1.08 (m, 3H), 1.29-1.43 (m, 1H), 1.71-1.97 (m, 2H), 2.04-2.32 (m, 2H), 2.76-3.09 (m, 1H), 3.69-3.99 (m, 1H), 4.00-4.07 (m, 3H), 5.43-5.76 (m, 1H), 7.33-7.44 (m, 1H), 7.56-7.66 (m, 1H), 7.69-7.76 (m, 1H), 7.89-8.00 (m, 1H), 8.00-8.03 (m, 1H), 8.44-8.77 (m, 2H), 10.85-11.07 (m, 1H), 12.07-12.74 (m, 1H), 13.86-14.04 (m, 1H).

LCMS(ESI): [M]⁺ m/z: calcd 432.2; found 433.2; Rt=2.146 min.

### Example 144. N-(4-amino-1H-pyrazolo[4,3-c]pyridin-7-yl)-2-((2R,5S)-2-(4-((dimethylamino)methyl)phenyl)-5-methylpiperidin-1-yl)-2-oxoacetamide (Compound 154)

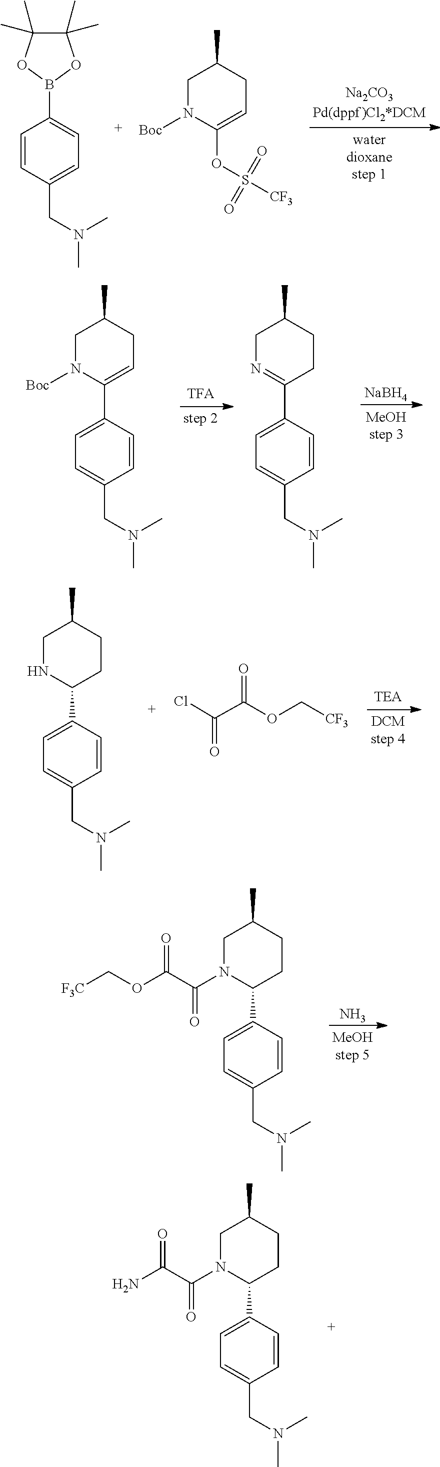

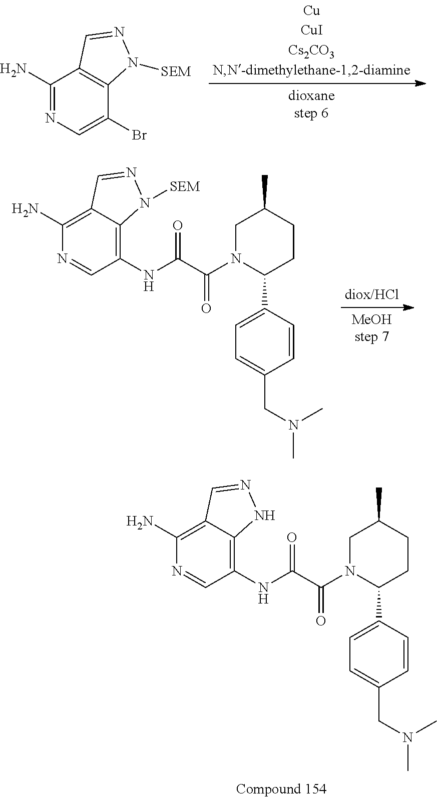

### Step 1: Synthesis of (S)-tert-butyl 6-(4-((dimethylamino)methyl)phenyl)-3-methyl-3,4-dihydropyridine-1(2H)-carboxylate

Prepared by general procedure scheme S8 step 2. Yield: 2.8 g of crude.

LCMS(ESI): [M]⁺ m/z: calcd 330.2; found 331.2; Rt=2.392 min.

### Step 2: Synthesis of (S)—N,N-dimethyl-1-(4-(5-methyl-3,4,5,6-tetrahydropyridin-2-yl)phenyl)methanamine

Prepared by general procedure scheme S8 step 3. Yield: 1.6 g of crude.

LCMS(ESI): [M]⁺ m/z: calcd 230.2; found 231.2; Rt=0.496 min.

### Step 3: Synthesis of N,N-dimethyl-1-(4-((2R,5S)-5-methylpiperidin-2-yl)phenyl)methanamine

Prepared by general procedure scheme S8 step 4. Yield: 0.5 g (30.98%).

CC conditions: The crude product was purified by silica gel with EtOAc+TEA/MeOH+TEA as an eluent mixture.

LCMS(ESI): [M]⁺ m/z: calcd 232.2; found 233.2; Rt=0.205 min.

### Step 4: Synthesis of 2,2,2-trifluoroethyl 2-((2R,5S)-2-(4-((dimethylamino)methyl)phenyl)-5-methylpiperidin-1-yl)-2-oxoacetate

Prepared by general procedure scheme S9 step 1. Yield: 0.8 g of crude.

LCMS(ESI): [M]⁺ m/z: calcd 386.2; found 387.2; Rt=2.123 min.

### Step 5: Synthesis of 2-((2R,5S)-2-(4-((dimethylamino)methyl)phenyl)-5-methylpiperidin-1-yl)-2-oxoacetamide

Ammonia (35.26 mg, 2.07 mmol) was bubbled through a solution of 2,2,2-trifluoroethyl 2-oxo-2-[(2R,5S)-2-[4-[(dimethylamino)methyl]phenyl]-5-methyl-1-piperidyl]acetate (0.8 g, 2.07 mmol) in MeOH (25 mL) at rt. After stirring for 18 hr, the reaction mixture was evaporated to dryness and purified by HPLC (25-50% 0.5-6.5 min; 30 ml/min water-MeCN+NH₃ (loading pump 4 ml/min MeCN+NH₃); column XBridge 19*100 mm (R)) to give 2-oxo-2-[(2R,5S)-2-[4-[(dimethylamino)methyl]phenyl]-5-methyl-1-piperidyl]acetamide (276 mg, 909.70 μmol, 43.94% yield) as a yellow gum.

LCMS(ESI): [M]⁺ m/z: calcd 303.2; found 304.2; Rt=0.544 min.

### Step 6: Synthesis of N-(4-amino-1-((2-(trimethylsilyl)ethoxy)methyl)-1H-pyrazolo[4,3-c]pyridin-7-yl)-2-((2R,5S)-2-(4-((dimethylamino)methyl)phenyl)-5-methylpiperidin-1-yl)-2-oxoacetamide

Prepared by general procedure scheme S9 step 3A. Yield: 111 mg (22.05%).

HPLC conditions: Column: Chromatorex C18 100*19 mm, 5 microM; 0-1-5 min 10-10-35% water-MeCN+0.1% FA 30 ml/min; (loading pump 4 ml/min MeCN).

LCMS(ESI): [M]⁺ m/z: calcd 565.2; found 566.2; Rt=0.927 min.

### Step 7: Synthesis of N-(4-amino-1H-pyrazolo[4,3-c]pyridin-7-yl)-2-((2R,5S)-2-(4-((dimethylamino)methyl)phenyl)-5-methylpiperidin-1-yl)-2-oxoacetamide (Compound 154)

To a solution of N-[4-amino-1-(2-trimethylsilylethoxymethyl)pyrazolo[4,3-c]pyridin-7-yl]-2-oxo-2-[(2R,5S)-2-[4-[(dimethylamino)methyl]phenyl]-5-methyl-1-piperidyl]acetamide (88.69 mg, 156.76 μmol) in MeOH (2 mL) was added hydrogen chloride solution 4.0M in dioxane (857.33 mg, 2.35 mmol, 1.07 mL, 10% purity) at 21° C. The resulting mixture was left to stir for 18 hr. The resulting mixture was evaporated to dryness. The residue was purified by RP-HPLC (column: Chromatorex 18 SMB100-5T 100×19 mm 5 um; 0-0-25% 0-2-5 min H₂O/MeCN/0.2% FA, flow: 30 ml/min) to give Compound 154 N-(4-amino-1H-pyrazolo[4,3-c]pyridin-7-yl)-2-oxo-2-[(2R,5S)-2-[4-[(dimethylamino)methyl]phenyl]-5-methyl-1-piperidyl]acetamide (16.5 mg, 31.28 μmol, 19.95% yield, 2HCOOH).

Compound 154: ¹H NMR (600 MHZ, DMSO-d₆) δ (ppm) 0.91-1.08 (m, 3H), 1.34 (s, 1H), 1.67 (s, 1H), 1.89 (d, 1H), 2.03 (s, 6H), 2.74 (d, 3H), 3.91 (dd, 3H), 4.95-5.68 (m, 1H), 6.27-6.85 (m, 2H), 7.35 (d, 4H), 7.50-7.79 (m, 1H), 8.19 (d, 1H), 9.51-10.59 (m, 1H), 13.02 (d, 1H).

LCMS(ESI): [M]⁺ m/z: calcd 435.2; found 436.2; Rt=1.776 min.

### Example 145. 2-((2R,5S)-2-(2-((1,3-dimethylpiperidin-4-yl)methyl)benzo[d]thiazol-5-yl)-5-methylpiperidin-1-yl)-2-oxo-N-(1H-pyrazolo[4,3-c]pyridin-7-yl)acetamide (Compound 156)

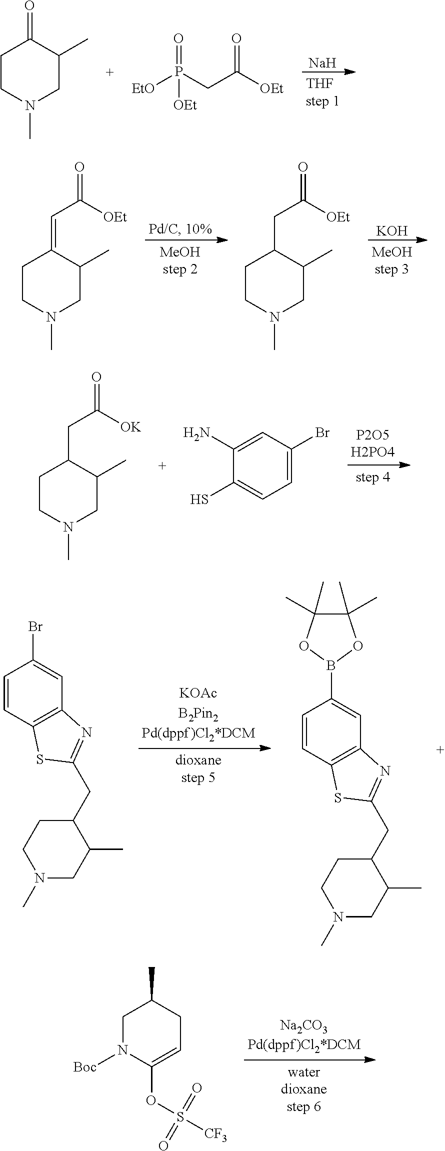

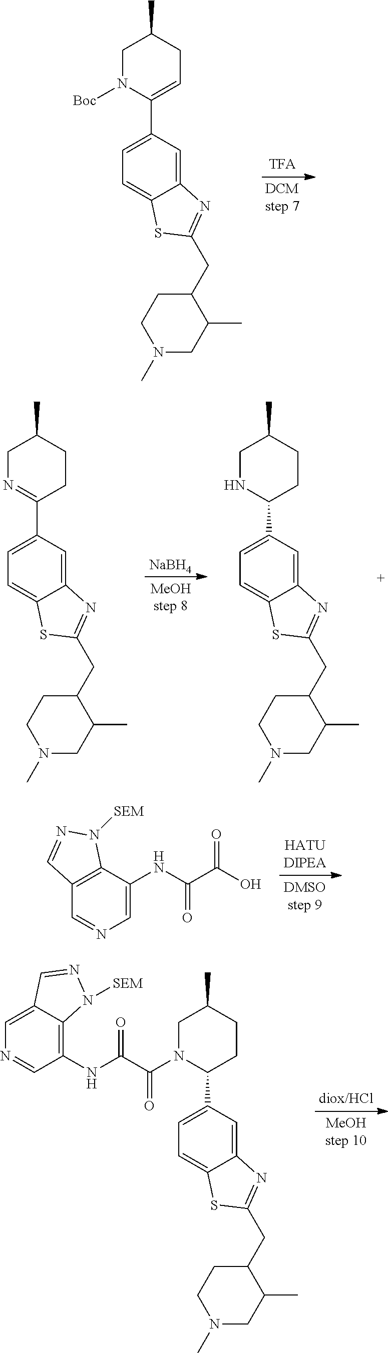

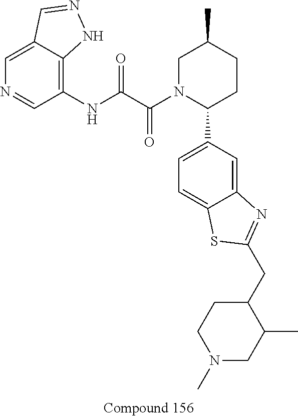 [SMILES: CC1CN(C)CCC1Cc1nc2cc([C@H]3CC[C@H](C)CN3C(=O)C(=O)Nc3cncc4cn[nH]c34)ccc2s1]

### Step 1: Synthesis of (E)-ethyl 2-(1,3-dimethylpiperidin-4-ylidene)acetate

To a stirred solution of sodium hydride (in oil dispersion) 60% dispersion in mineral oil (1.90 g, 82.56 mmol) in THF (300 mL) was added dropwise ethyl 2-(diethoxyphosphoryl)acetate (19.39 g, 86.49 mmol, 17.24 mL), then THE solution of 1,3-dimethylpiperidin-4-one (10 g, 78.63 mmol) was added after 1 hr. The resulting reaction mixture was stirred at 25° C. for 12 hr. Upon completion, the reaction mixture concentrated under reduced pressure. Then it was extracted with MTBE/water (150 ml/30 ml), organic layer was dried over Na₂SO₄ and evaporated. The desired product ethyl (2E)-2-(1,3-dimethyl-4-piperidylidene)acetate (13.5 g, 68.43 mmol, 87.04% yield) was isolated as yellow liquid.

¹H NMR (500 MHz, CDCl₃) δ (ppm) 1.07 (d, 3H), 1.25 (t, 3H), 1.90 (m, 1H), 2.01 (m, 1H), 2.21 (s, 3H), 2.48 (m, 2H), 2.73 (m, 2H), 3.54 (m, 1H), 4.14 (m, 2H), 5.60 (s, 1H).

### Step 2: Synthesis of ethyl 2-(1,3-dimethylpiperidin-4-yl)acetate

To a stirred solution of ethyl (2E)-2-(1,3-dimethyl-4-piperidylidene)acetate (6 g, 30.41 mmol) in MeOH (50 mL) palladium, 10% on carbon (0.5 g, 4.70 mmol) was added at 25° C. The resulting reaction mixture was vacuumed and stirred at 25° C. for 16 hr under hydrogen (61.31 mg, 30.41 mmol). The reaction mixture was filtered off and concentrated in vacuum to give ethyl 2-(1,3-dimethyl-4-piperidyl)acetate (5.48 g, crude). GCMS: [M]: calcd 199.2; found 199.2; Rt=6.739 min.

### Step 3: Synthesis of potassium 2-(1,3-dimethylpiperidin-4-yl)acetate

To a stirred solution of ethyl 2-(1,3-dimethyl-4-piperidyl)acetate (5.48 g, 27.50 mmol) in MeOH (40.04 mL) were added potassium hydroxide (2.31 g, 41.25 mmol, 1.13 mL) respectively at 25° C. The resulting reaction mixture was stirred at 25° C. for 12 hr, next concentrated in vacuum to give potassium; 2-(1,3-dimethyl-4-piperidyl)acetate (8 g, crude). GCMS: [M]: calcd 209.2; found 209.2; Rt=2.211 min.

### Step 4: Synthesis of 5-bromo-2-((1,3-dimethylpiperidin-4-yl)methyl)benzo[d]thiazole

To a stirred H₃PO₄ (27.75 g, 240.70 mmol, 15 mL, 85% purity) P₂O₅ (4.5 g, 31.70 mmol) was added at 60° C. and next heated to 120° C. The resulting reaction mixture was stirred at 120° C. for 30 min. Then potassium; 2-(1,3-dimethyl-4-piperidyl)acetate (7.5 g, 21.50 mmol) and 2-amino-4-bromo-benzenethiol (4.39 g, 21.50 mmol) were added and stirred overnight. Upon completion, the reaction mixture was cooled to rt. Then solution of NaOH with ice was added, and quenched to pH=14. Desired product was extracted with EtOAc. Organic layer was washed with water twice, then dried over Na₂SO₄ and evaporated to give 5-bromo-2-[(1,3-dimethyl-4-piperidyl)methyl]-1,3-benzothiazole (5.5 g, crude).

LCMS(ESI): [M]⁺ m/z: calcd 339.2; found 340.2; Rt=0.933 min.

### Step 5: Synthesis of 2-((1,3-dimethylpiperidin-4-yl)methyl)-5-(4,4,5,5-tetramethyl-1,3,2-dioxaborolan-2-yl)benzo[d]thiazole

Prepared by general procedure scheme S2 step 2. Yield: 3.8 g of crude.

LCMS(ESI): [M]⁺ m/z: calcd 386.2; found 387.2; Rt=1.010 min.

### Step 6: Synthesis of (3S)-tert-butyl 6-(2-((1,3-dimethylpiperidin-4-yl)methyl)benzo[d]thiazol-5-yl)-3-methyl-3,4-dihydropyridine-1(2H)-carboxylate

Prepared by general procedure scheme S2 step 3. Yield: 3.8 g of crude.

LCMS(ESI): [M]⁺ m/z: calcd 455.2; found 456.2; Rt=1.171 min.

### Step 7: Synthesis of 2-((1,3-dimethylpiperidin-4-yl)methyl)-5-((S)-5-methyl-3,4,5,6-tetrahydropyridin-2-yl)benzo[d]thiazole

To a solution of tert-butyl (3S)-6-[2-[(1,3-dimethyl-4-piperidyl)methyl]-1,3-benzothiazol-5-yl]-3-methyl-3,4-dihydro-2H-pyridine-1-carboxylate (3.8 g, 4.17 mmol) in DCM (10 mL) TFA (15 g, 131.55 mmol, 10.14 mL) was added and then stirred for 6 hr at 20° C. The reaction mixture was concentrated in vacuum, treated with water and DCM. Aq. layer was washed with DCM twice. Aq. solution of NaHCO₃ was added to basic pH of solution, desired product was extracted with DCM (2*50 ml). Organic layer was dried over Na₂SO₄ and then concentrated in vacuum to give 2-[(1,3-dimethyl-4-piperidyl)methyl]-5-[(3S)-3-methyl-2,3,4,5-tetrahydropyridin-6-yl]-1,3-benzothiazole (0.8 g, crude).

LCMS(ESI): [M]⁺ m/z: calcd 355.2; found 356.2; Rt=0.542 min.

### Step 8: Synthesis of 2-((1,3-dimethylpiperidin-4-yl)methyl)-5-((2R,5S)-5-methylpiperidin-2-yl)benzo[d]thiazole

Prepared by general procedure scheme S2 step 5. Yield: 0.51 g of crude.

LCMS(ESI): [M]⁺ m/z: calcd 357.2; found 358.2; Rt=0.484 min.

### Step 9: Synthesis of 2-((2R,5S)-2-(2-((1,3-dimethylpiperidin-4-yl)methyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6B. Yield: 84.9 mg (26.42%).

HPLC conditions: Column: SunFire C18 100*19 mm, 5 microM; 2-10 min 30-65%

MeOH; (loading pump 4 ml/min MeOH).

LCMS(ESI): [M]⁺ m/z: calcd 675.2; found 676.2; Rt=3.129 min.

### Step 10: Synthesis of 2-((2R,5S)-2-(2-((1,3-dimethylpiperidin-4-yl)methyl)benzo[d]thiazol-5-yl)-5-methylpiperidin-1-yl)-2-oxo-N-(1H-pyrazolo[4,3-c]pyridin-7-yl) Compound 156)

2-Oxo-2-[(2R,5S)-2-[2-[(1,3-dimethyl-4-piperidyl)methyl]-1,3-benzothiazol-5-yl]-5-methyl-1-piperidyl]-N-[1-(2-trimethylsilylethoxymethyl)pyrazolo[4,3-c]pyridin-7-yl]acetamide (84.9 mg, 125.60 μmol) was dissolved in dioxane (3 mL) saturated with HCl (10% by weight). MeOH (1 mL) was added for better solubility. The reaction mixture was stirred for 1 hr at 20° C. then concentrated in vacuum. The residue was dissolved in 1 ml of MeOH and subjected to HPLC (Device (Mobile Phase, Column): 2-10 min 30-65% MeCN+FA, 30 ml/min). 2-Oxo-N-(1H-pyrazolo[4,3-c]pyridin-7-yl)-2-[(2R,5S)-2-[2-[(1,3-dimethyl-4-piperidyl)methyl]-1,3-benzothiazol-5-yl]-5-methyl-1-piperidyl]acetamide (33.9 mg, 57.29 μmol, 45.61% yield, HCOOH) was obtained.

Compound 156: ¹H NMR (600 MHZ, DMSO-d₆) δ (ppm) 0.75-1.06 (m, 6H), 1.37-1.50 (m, 4H), 1.74-2.33 (m, 8H), 2.73-3.02 (m, 6H), 3.78-4.12 (m, 2H), 5.58-5.75 (m, 1H), 7.39-7.43 (m, 1H), 7.90-8.15 (m, 2H), 8.30-8.54 (m, 2H), 8.90-8.96 (m, 1H), 11.14-11.24 (m, 1H), 13.09-13.16 (m, 1H).

LCMS(ESI): [M]⁺ m/z: calcd 545.2; found 546.2; Rt=1.792 min.

### Example 146. 2-((2R,5S)-2-(2-(1-(dimethylamino)ethyl)benzo[d]thiazol-5-yl)-5-methylpiperidin-1-yl)-2-oxo-N-(1H-pyrazolo[4,3-c]pyridin-7-yl)acetamide (Compound 153 and Compound 167)

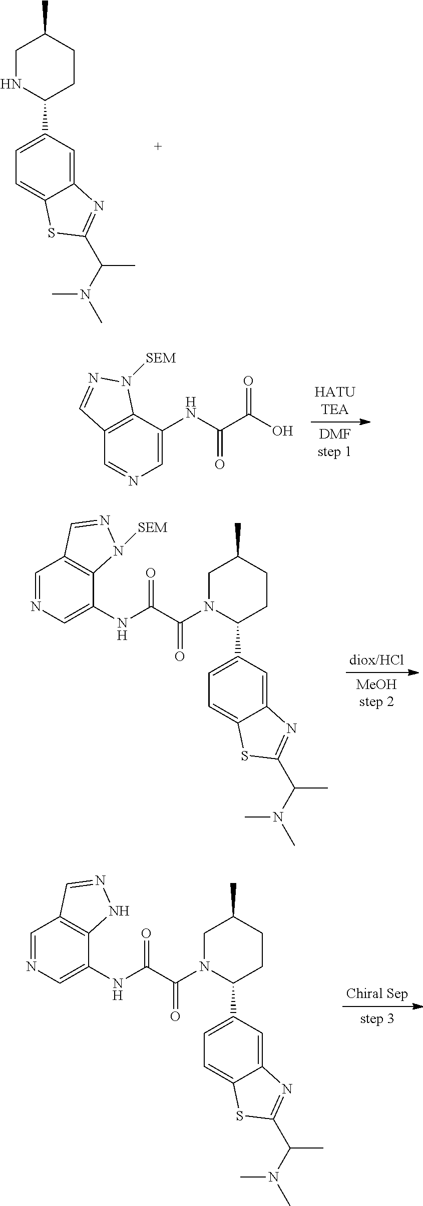 [SMILES: CC(c1nc2cc([C@H]3CC[C@H](C)CN3)ccc2s1)N(C)C ; C[Si](C)(C)CCOCn1ncc2cncc(NC(=O)C(=O)O)c21 ; CC(c1nc2cc([C@H]3CC[C@H](C)CN3C(=O)C(=O)Nc3cncc4cnn(COCC[Si](C)(C)C)c34)ccc2s1)N(C)C ; CC(c1nc2cc([C@H]3CC[C@H](C)CN3C(=O)C(=O)Nc3cncc4cn[nH]c34)ccc2s1)N(C)C]

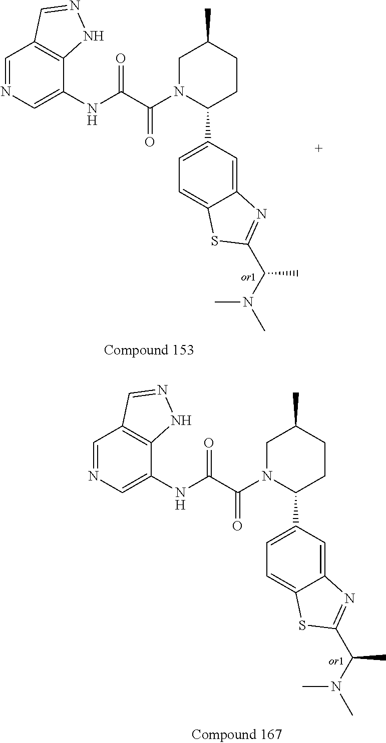 [SMILES: C[C@H]1CC[C@H](c2ccc3sc([C@H](C)N(C)C)nc3c2)N(C(=O)C(=O)Nc2cncc3cn[nH]c23)C1 ; C[C@H]1CC[C@H](c2ccc3sc([C@@H](C)N(C)C)nc3c2)N(C(=O)C(=O)Nc2cncc3cn[nH]c23)C1]

The synthesis of N,N-dimethyl-1-(5-((2R,5S)-5-methylpiperidin-2-yl)benzo[d]thiazol-2-yl)ethanamine is given by Intermediate 45.

### Step 1: Synthesis of 2-((2R,5S)-2-(2-(1-(dimethylamino)ethyl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S4 step 6A. Yield: 132 mg (32.21%).

HPLC conditions: Column: Chromatorex C18 100*19 mm, 5 microM; 0-1-6 min 45-45-85% water-MeCN+0.1% NH₄OH, flow: 30 ml/min; (loading pump 4 ml/min MeCN).

LCMS(ESI): [M]⁺ m/z: calcd 621.2; found 622.2; Rt=1.046 min.

### Step 2: Synthesis of 2-((2R,5S)-2-(2-(1-(dimethylamino)ethyl)benzo[d]thiazol-5-yl)-5-methylpiperidin-1-yl)-2-oxo-N-(1H-pyrazolo[4,3-c]pyridin-7-yl)acetamide

Hydrogen chloride solution 4.0M in dioxane (1.01 g, 2.77 mmol, 1 mL, 10% purity) was added to the solution of 2-oxo-2-[(2R,5S)-2-[2-[1-(dimethylamino)ethyl]-1,3-benzothiazol-5-yl]-5-methyl-1-piperidyl]-N-[1-(2-trimethylsilylethoxymethyl)pyrazolo[4,3-c]pyridin-7-yl]acetamide (132 mg, 212.26 μmol) in MeOH (2 mL). Resulting mixture was stirred at 25° C. for 72 hr. Then, volatiles were removed under reduced pressure and residue was purified by HPLC (30-80% 0-5 min H₂O/MeOH/0.1% NH₄OH, flow: 30 ml/min; column: XBridge C18 100×19 mm, 5 um), affording 2-oxo-N-(1H-pyrazolo[4,3-c]pyridin-7-yl)-2-[(2R,5S)-2-[2-[1-(dimethylamino)ethyl]-1,3-benzothiazol-5-yl]-5-methyl-1-piperidyl]acetamide (43 mg, 87.47 μmol, 41.21% yield).

LCMS(ESI): [M]⁺ m/z: calcd 491.2; found 492.2; Rt=0.667 min.

### Step 3: Chiral Separation (Compound 153 and Compound 167)

Racemic 2-((2R,5S)-2-(2-(1-(dimethylamino)ethyl)benzo[d]thiazol-5-yl)-5-methylpiperidin-1-yl)-2-oxo-N-(1H-pyrazolo[4,3-c]pyridin-7-yl)acetamide (43.00 mg, 87.47 μmol) was chiral separated (Column: Chiralcel OD-H (250×20 mm, 5 um); Mobile phase: Hexane:IPA:MeOH, 90:5:5 Flow Rate: 12 mL/min) to obtain 2-((2R,5S)-2-(2-((S)-1-(dimethylamino)ethyl)benzo[d]thiazol-5-yl)-5-methylpiperidin-1-yl)-2-oxo-N-(1H-pyrazolo[4,3-c]pyridin-7-yl)acetamide (18 mg, 36.61 μmol, 83.72% yield) (RT=30.37 min) and 2-((2R,5S)-2-(2-((R)-1-(dimethylamino)ethyl)benzo[d]thiazol-5-yl)-5-methylpiperidin-1-yl)-2-oxo-N-(1H-pyrazolo[4,3-c]pyridin-7-yl)acetamide (18.8 mg, 38.24 μmol, 87.44% yield) (RT=39.98 min).

Rel Time for Compound 153 in analytical conditions (column: OD-H, Hexane-IPA-MeOH, 90-5-5, 1 ml/min as mobile phase) 26.76 min and for Compound 167 35.82 min.

Compound 153: Retention time: 26.76 min.

¹H NMR (600 MHz, DMSO-d₆) δ 1.03-1.09 (m, 3H), 1.34-1.44 (m, 4H), 1.71-1.81 (m, 1H), 1.86-2.02 (m, 1H), 2.08-2.23 (m, 1H), 2.25-2.29 (m, 6H), 2.30-2.36 (m, 1H), 2.87-3.27 (m, 1H), 3.75-4.15 (m, 2H), 5.56-5.79 (m, 1H), 7.35-7.46 (m, 1H), 7.88-7.93 (m, 1H), 7.98-8.09 (m, 1H), 8.26-8.38 (m, 1H), 8.40-8.55 (m, 1H), 8.87-9.01 (m, 1H), 10.94-11.31 (m, 1H), 12.92-13.23 (m, 1H).

LCMS(ESI): [M]⁺ m/z: calcd 491.2; found 492.2; Rt=0.876 min.

Compound 167: Retention time: 35.82 min.

¹H NMR (600 MHz, DMSO-d₆) δ 1.04-1.10 (m, 3H), 1.36-1.43 (m, 4H), 1.69-1.82 (m, 1H), 1.87-1.97 (m, 1H), 2.09-2.22 (m, 1H), 2.25-2.28 (m, 6H), 2.30-2.36 (m, 1H), 2.88-3.26 (m, 1H), 3.80-4.16 (m, 2H), 5.50-5.85 (m, 1H), 7.35-7.48 (m, 1H), 7.86-7.94 (m, 1H), 7.99-8.09 (m, 1H), 8.24-8.37 (m, 1H), 8.37-8.61 (m, 1H), 8.89-9.01 (m, 1H), 10.94-11.30 (m, 1H), 12.89-13.14 (m, 1H).

LCMS(ESI): [M]⁺ m/z: calcd 491.2; found 492.2; Rt=0.875 min.

### Example 147. N-(imidazo[1,2-a]pyridin-7-yl)-2-((2R,5S)-5-methyl-2-(2-(1-methyl-1,2,3,6-tetrahydropyridin-4-yl)benzo[d]thiazol-5-yl)piperidin-1-yl)-2-oxoacetamide (Compound 157)

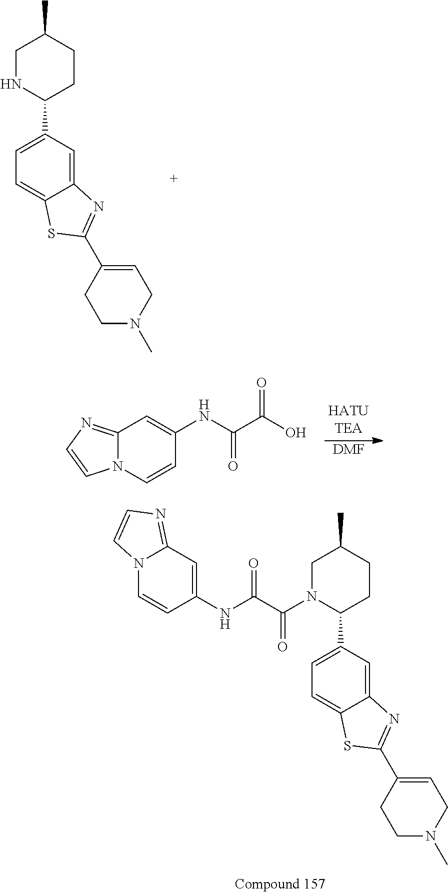

Synthesis of 2-(1-methyl-1,2,3,6-tetrahydropyridin-4-yl)-5-((2R,5S)-5-methylpiperidin-2-yl)benzo[d]thiazole is given by Intermediate 46.

Prepared by general procedure scheme S2 step 6A. Yield: 59.7 mg (23.74%).

HPLC conditions: Column: XBridge C18 100*19 mm, 5 microM; 0-1-6 min 40-40-75% water-MeOH+0.1% NH₄OH; (loading pump 4 ml/min MeOH).

Compound 157: ¹H NMR (600 MHz, DMSO-d₆) δ (ppm) 1.05 (m, 3H), 1.37 (m, 1H), 1.72 (m, 1H), 1.89 (m, 1H), 2.15 (m, 1H), 2.29 (m, 4H), 2.58 (m, 2H), 2.67 (m, 2H), 3.10 (m, 2H), 4.04 (m, 2H), 5.49 (m, 1H), 6.75 (m, 1H), 7.07 (m, 1H), 7.44 (m, 2H), 7.94 (m, 4H), 8.46 (m, 1H), 11.15 (m, 1H).

LCMS(ESI): [M]⁺ m/z: calcd 514.2; found 515.2; Rt=1.864 min.

### Example 148. N-(4-amino-1H-pyrazolo[4,3-c]pyridin-7-yl)-2-((2R,5S)-2-(3-((dimethylamino)methyl)phenyl)-5-methylpiperidin-1-yl)-2-oxoacetamide (Compound 158)

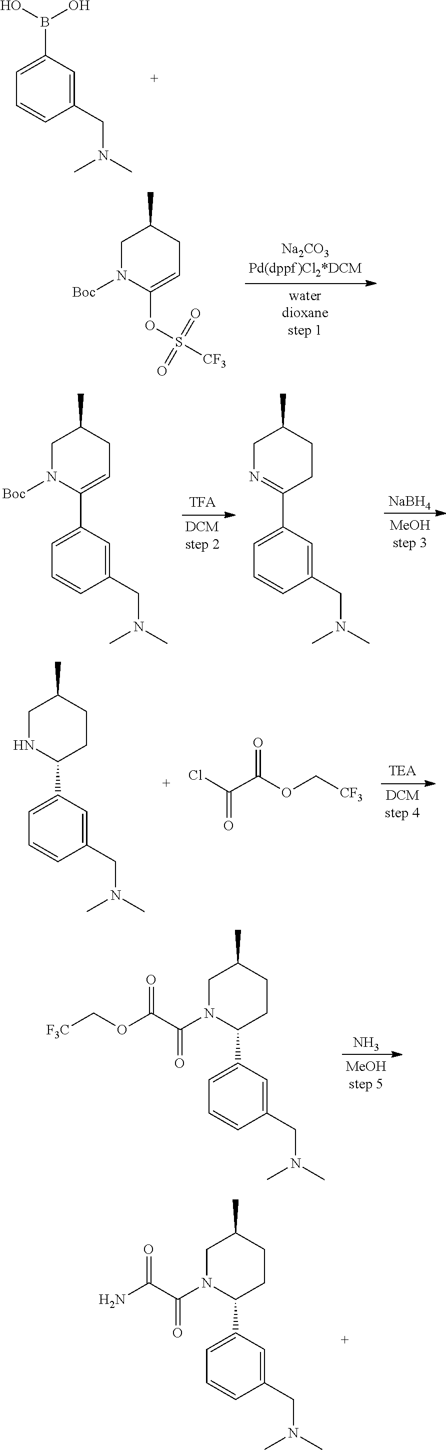

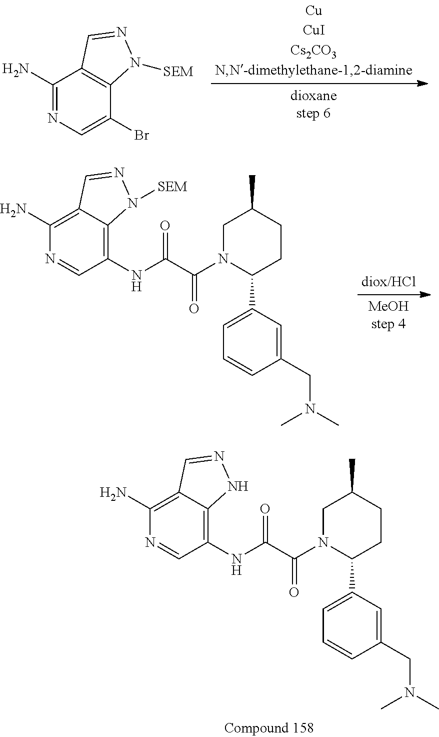

### Step 1: Synthesis of (S)-tert-butyl 6-(3-((dimethylamino)methyl)phenyl)-3-methyl-3,4-dihydropyridine-1(2H)-carboxylate

Prepared by general procedure scheme S8 step 2. Yield: 3 g of crude.

LCMS(ESI): [M]⁺ m/z: calcd 330.2; found 331.2; Rt=2.407 min.

### Step 2: Synthesis of (S)—N,N-dimethyl-1-(3-(5-methyl-3,4,5,6-tetrahydropyridin-2-yl)phenyl)methanamine

Prepared by general procedure scheme S8 step 3. Yield: 1.5 g of crude.

LCMS(ESI): [M]⁺ m/z: calcd 230.2; found 231.2; Rt=0.303 min.

### Step 3: Synthesis of N,N-dimethyl-1-(3-((2R,5S)-5-methylpiperidin-2-yl)phenyl)methanamine

Prepared by general procedure scheme S8 step 4. Yield: 0.26 g (17.18%).

CC conditions: The crude product was purified by silica gel with MTBE/MeOH, 0-100% as an eluent mixture.

LCMS(ESI): [M]⁺ m/z: calcd 232.2; found 233.2; Rt=0.268 min.

### Step 4: Synthesis of 2,2,2-trifluoroethyl 2-((2R,5S)-2-(3-((dimethylamino)methyl)phenyl)-5-methylpiperidin-1-yl)-2-oxoacetate

Prepared by general procedure scheme S9 step 1. Yield: 0.43 g of crude.

LCMS(ESI): [M]⁺ m/z: calcd 386.2; found 387.2; Rt=2.204 min.

### Step 5: Synthesis of 2-((2R,5S)-2-(3-((dimethylamino)methyl)phenyl)-5-methylpiperidin-1-yl)-2-oxoacetamide

Ammonia (18.95 mg, 1.11 mmol) was bubbled through a solution of 2,2,2-trifluoroethyl 2-oxo-2-[(2R,5S)-2-[3-[(dimethylamino)methyl]phenyl]-5-methyl-1-piperidyl]acetate (0.43 g, 1.11 mmol) in MeOH (15 mL) at rt. After stirring for 18 hr, the reaction mixture was evaporated to dryness and purified by HPLC (0-30% 0.5-6.5 min; 30 ml/min water-MeCN+NH₃ (loading pump 4 ml/min MeCN+NH₃); column XBridge 19*100 mm (R)) to give 2-oxo-2-[(2R,5S)-2-[3-[(dimethylamino)methyl]phenyl]-5-methyl-1-piperidyl]acetamide (104.5 mg, 344.43 μmol, 30.95% yield) as a yellow gum.

LCMS(ESI): [M]⁺ m/z: calcd 303.2; found 304.2; Rt=0.569 min.

### Step 6: Synthesis of N-(4-amino-1-((2-(trimethylsilyl)ethoxy)methyl)-1H-pyrazolo[4,3-c]pyridin-7-yl)-2-((2R,5S)-2-(3-((dimethylamino)methyl)phenyl)-5-methylpiperidin-1-yl)-2-oxoacetamide

Prepared by general procedure scheme S9 step 3A. Yield: 68 mg (34.73%).

HPLC conditions: Column: Chromatorex C18 100*19 mm, 5 microM; 0-1-5 min 10-10-35% water-MeCN+0.1% FA 30 ml/min; (loading pump 4 ml/min MeCN).

LCMS(ESI): [M]⁺ m/z: calcd 565.2; found 566.2; Rt=0.931 min.

### Step 7: Synthesis of N-(4-amino-1H-pyrazolo[4,3-c]pyridin-7-yl)-2-((2R,5S)-2-(3-((dimethylamino)methyl)phenyl)-5-methylpiperidin-1-yl)-2-oxoacetamide (Compound 158)

To a solution of N-[4-amino-1-(2-trimethylsilylethoxymethyl)pyrazolo[4,3-c]pyridin-7-yl]-2-oxo-2-[(2R,5S)-2-[3-[(dimethylamino)methyl]phenyl]-5-methyl-1-piperidyl]acetamide (88.69 mg, 156.76 μmol) in MeOH (2 mL) was added hydrogen chloride solution 4.0M in dioxane (857.33 mg, 2.35 mmol, 1.07 mL, 10% purity) at 21° C. The resulting mixture was left to stir for 18 hr. The resulting mixture was evaporated to dryness. The residue was purified by RP-HPLC (column: Chromatorex 18 SMB100-5T 100×19 mm 5 um; 0-0-25% 0-2-5 min H₂O/MeCN/0.2% FA, flow: 30 ml/min, flow: 30 ml/min) to give Compound 158 N-(4-amino-1H-pyrazolo[4,3-c]pyridin-7-yl)-2-oxo-2-[(2R,5S)-2-[3-[(dimethylamino)methyl]phenyl]-5-methyl-1-piperidyl]acetamide (13.8 mg, 26.16 μmol, 16.69% yield, 2HCOOH).

Compound 158: ¹H NMR (600 MHZ, DMSO-d₆) δ (ppm) 0.92-1.06 (m, 3H), 1.30-1.42 (m, 1H), 1.66-1.73 (m, 1H), 1.86-1.94 (m, 1H), 2.02-2.12 (m, 1H), 2.18-2.27 (m, 2H), 2.83-3.06 (m, 2H), 3.22-3.27 (m, 6H), 3.73-4.13 (m, 1H), 5.45-5.66 (m, 1H), 6.46-7.05 (m, 2H), 7.07-7.56 (m, 4H), 7.63-7.80 (m, 1H), 8.14-8.30 (m, 1H), 10.41-10.59 (m, 1H), 12.30-13.07 (m, 1H).

LCMS(ESI): [M]⁺ m/z: calcd 435.2; found 436.2; Rt=1.796 min.

### Example 149. 2-oxo-N-(1H-pyrazolo[4,3-c]pyridin-7-yl)-2-[rac-(2R,5S)-5-methyl-2-[2-[rac-(3S,4S)-1,3-dimethyl-4-piperidyl]indazol-6-yl]-1-piperidyl]acetamide (Compound 159)

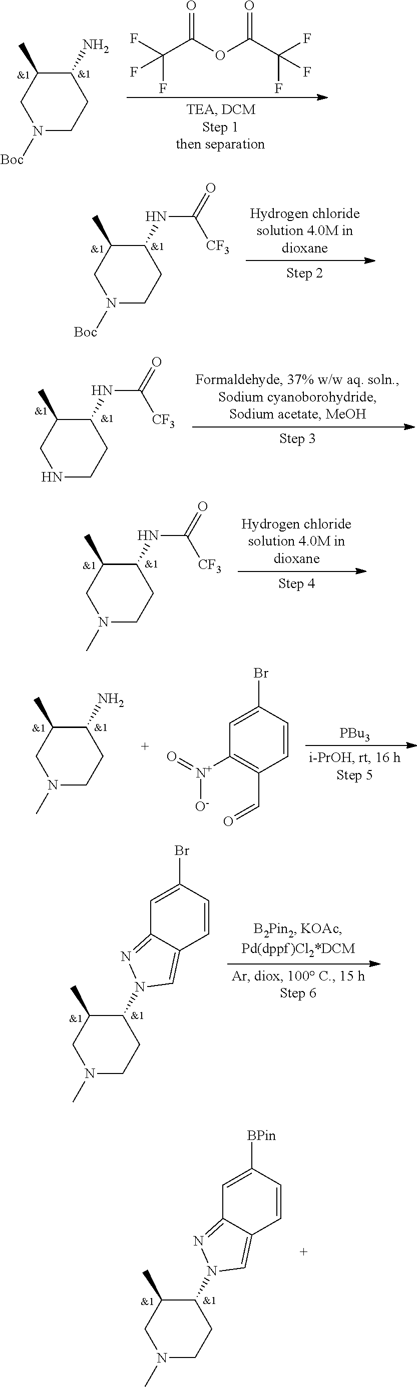

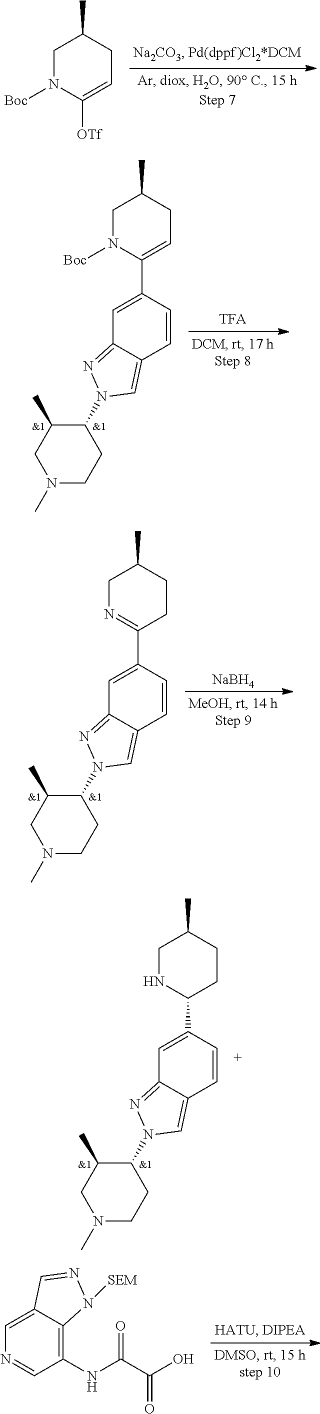

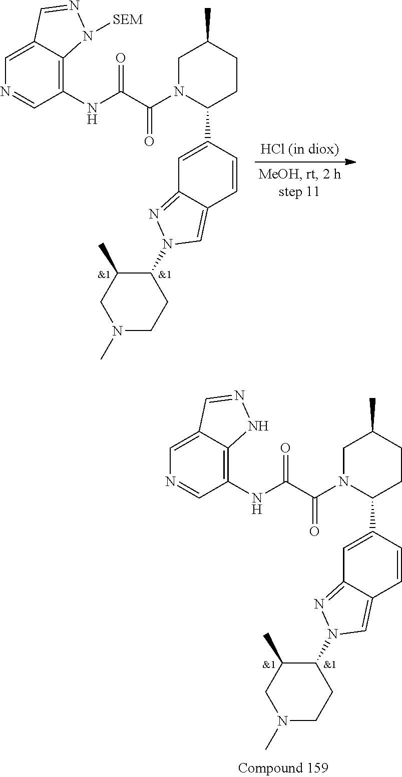 [SMILES: C[C@H]1CC[C@H](c2ccc3cn([C@@H]4CCN(C)C[C@H]4C)nc3c2)N(C(=O)C(=O)Nc2cncc3cnn(COCC[Si](C)(C)C)c23)C1 ; C[C@H]1CC[C@H](c2ccc3cn([C@@H]4CCN(C)C[C@H]4C)nc3c2)N(C(=O)C(=O)Nc2cncc3cn[nH]c23)C1]

### Step 1: The Synthesis of tert-butyl rac-(3S,4S)-3-methyl-4-[(2,2,2-trifluoroacetyl)amino]piperidine-1-carboxylate and tert-butyl rac-(3S,4R)-3-methyl-4-[(2,2,2-trifluoroacetyl)amino]piperidine-1-carboxylate

To a solution of tert-butyl 4-amino-3-methyl-piperidine-1-carboxylate (15.15 g, 70.69 mmol) and TEA (10.73 g, 106.04 mmol, 14.78 mL) in DCM (250 mL) was added TFAA (16.33 g, 77.76 mmol, 10.98 mL) at 0° C. After stirring at rt for 1 hr the resulting mixture was washed with NaHCO₃, 1NHCl, dried, and evaporated to dryness. The separate diastereoisomers tert-butyl rac-(3S,4R)-3-methyl-4-[(2,2,2-trifluoroacetyl)amino]piperidine-1-carboxylate (7.8 g, 25.14 mmol, 35.56% yield), and tert-butyl rac-(3S,4S)-3-methyl-4-[(2,2,2-trifluoroacetyl)amino]piperidine-1-carboxylate (9 g, 29.00 mmol, 41.03% yield) were obtained as white solids by column chromatography purification of the residue (the conditions are: Interchim, 330 g SiO2, petroleum ether/MtBE with MtBE from 0-30%, flow rate=127 mL/min, Rv=10.5-13.3 CV). 1.4 g of 1:1 mixture has been obtained as mixed fraction.

### Step 2: The Synthesis of 2,2,2-trifluoro-N-[rac-(3S,4S)-3-methyl-4-piperidyl]acetamide

To a solution of tert-butyl rac-(3S,4S)-3-methyl-4-[(2,2,2-trifluoroacetyl)amino]piperidine-1-carboxylate (9 g, 29.00 mmol) in Et2O (150 mL) was added Hydrogen chloride solution 4.0M in dioxane (80.00 g, 2.19 mol, 100 mL) at 21° C. The resulting mixture was left to stir for 48 hr. The resulting mixture was evaporated to dryness to give 2,2,2-trifluoro-N-[rac-(3S,4S)-3-methyl-4-piperidyl]acetamide (6.2 g, 25.14 mmol, 86.67% yield, HCl) as a white solid.

¹H NMR (500 MHz, dmso) δ 0.81 (d, 3H), 1.74-1.83 (m, 2H), 1.99 (m, 1H), 2.68 (m, 1H), 2.93 (m, 1H), 3.23 (dd, 2H), 3.65 (m, 1H), 9.13 (brs, 2H), 9.544 (d, 1H).

### Step 3: The Synthesis of 2,2,2-trifluoro-N-[rac-(3S,4S)-1,3-dimethyl-4-piperidyl]acetamide

Formaldehyde, 37% w/w aq. soln., stab. with 7-8% methanol (2.24 g, 27.65 mmol, 2.07 mL, 37% purity) and acetic acid (4.53 g, 75.41 mmol, 4.32 mL) were added to the solution of 2,2,2-trifluoro-N-[rac-(3S,4S)-3-methyl-4-piperidyl]acetamide (6.2 g, 25.14 mmol, HCl) and Sodium acetate, anhydrous (2.27 g, 27.65 mmol, 1.48 mL) in MeOH (45.68 mL). Resulting mixture was stirred at 21° C. for 1 hour before Sodium cyanoborohydride (3.16 g, 50.27 mmol) was added thereto. After that, stirring was continued for 18 hr. Then, the solvent was removed under reduced pressure, and residue was partitioned between sat. aq. NaHCO₃ solution (50 ml) and EtOAc (50 mL). Water layer was extracted with EtOAc (3×50 mL). The organic layers were combined and evaporated to give 2,2,2-trifluoro-N-[rac-(3S,4S)-1,3-dimethyl-4-piperidyl]acetamide (5.6 g, 24.98 mmol, 99.36% yield) as a white solid.

¹H NMR (400 MHZ, CDCl₃) δ 0.914 (d, 3H), 1.57 (m, 1H), 1.68 (m, 1H), 1.80 (m, 1H), 1.98-2.05 (m, 2H), 2.28 (s, 3H), 2.87 (m, 2H), 3.54 (m, 1H), 6.13 (brs, 1H).

### Step 4: The Synthesis of rac-(3S,4S)-1,3-dimethylpiperidin-4-amine

To a solution of 2,2,2-trifluoro-N-[rac-(3S,4S)-1,3-dimethyl-4-piperidyl]acetamide (5.6 g, 24.98 mmol) in MeOH (25 mL) was added Hydrogen chloride solution 36% in water (910.62 mg, 24.98 mmol, 25 mL) at 21° C. The resulting mixture was heated to 80° C. and left to stir for 48 hr. The resulting mixture was evaporated to dryness. The solid rac-(3S,4S)-1,3-dimethylpiperidin-4-amine (4.9 g, 24.36 mmol, 97.54% yield) was used in the next step without further purification.

¹H NMR (400 MHZ, DMSO-d₆) δ 0.95 (d, 3H), 1.95 (m, 1H), 2.19 (m, 2H), 2.66 (s, 3H), 2.76 (m, 1H), 2.95 (m, 2H), 3.34 (m, 1H), 8.43 (brs, 3H).

LCMS(ESI): [M+H]⁺ m/z: calcd 128.1; found 129.0; Rt=0.147 min.

### Step 5: The Synthesis of 6-bromo-2-[rac-(3S,4S)-1,3-dimethyl-4-piperidyl]indazole

### Prepared by General Procedure Scheme S7 Step 1A

Yield: 4.5 g (59.93%)

¹H NMR (500 MHz, DMSO-d₆) δ 0.52 (d, 3H), 1.74 (t, 1H), 1.89 (d, 1H), 2.02 (t, 3H), 2.16 (m, 1H), 2.20 (s, 3H), 2.27 (m, 1H), 2.88 (t, 2H), 4.05 (t, 1H), 7.10 (d, 1H), 7.62 (d, 1H), 7.86 (s, 1H), 8.46 (s, 1H).

### Step 6: The Synthesis of 2-[rac-(3S,4S)-1,3-dimethyl-4-piperidyl]-6-(4,4,5,5-tetramethyl-1,3,2-dioxaborolan-2-yl)indazole

### Prepared by General Procedure Scheme S7 Step 2A

Yield: 7.5 g (crude)

LCMS(ESI): [M+H]⁺ m/z: calcd 355.2; found 356.4; Rt=2.514 min.

### Step 7: The Synthesis of tert-butyl rac-(3S)-3-methyl-6-[2-[rac-(3S,4S)-1,3-dimethyl-4-piperidyl]indazol-6-yl]-3,4-dihydro-2H-pyridine-1-carboxylate

### Prepared by General Procedure Scheme S7 Step 2

Yield: 9.0 g (crude)

LCMS(ESI): [M+H]⁺ m/z: calcd 424.3; found 425.6; Rt=3.354 min.

### Step 8: The Synthesis of 2-[rac-(3S,4S)-1,3-dimethyl-4-piperidyl]-6-[rac-(3S)-3-methyl-2,3,4,5-tetrahydropyridin-6-yl]indazole

### Prepared by General Procedure Scheme S7 Step 3 (Method A)

Yield: 8.85 g (crude)

LCMS(ESI): [M+H]⁺ m/z: calcd 324.2; found 325.2; Rt=1.485 min.

### Step 9: The Synthesis of 2-[rac-(3S,4S)-1,3-dimethyl-4-piperidyl]-6-[rac-(2R,5S)-5-methyl-2-piperidyl]indazole

### Prepared by General Procedure Scheme S7 Step 4

Yield: 180 mg (10.11%)

LCMS(ESI): [M+H]⁺ m/z: calcd 326.2; found 327.4; Rt=0.451 min.

### Step 10: The Synthesis of 2-oxo-2-[rac-(2R,5S)-5-methyl-2-[2-[rac-(3S,4S)-1,3-dimethyl-4-piperidyl]indazol-6-yl]-1-piperidyl]-N-[1-(2-trimethylsilylethoxymethyl)pyrazolo[4,3-c]pyridin-7-yl]acetamide

### Prepared by General Procedure Scheme S7 Step 5

Yield: 110 mg (crude)

LCMS(ESI): [M+H]⁺ m/z: calcd 644.4; found 645.2; Rt=3.16 min.

### Step 11: The Synthesis of 2-oxo-N-(1H-pyrazolo[4,3-c]pyridin-7-yl)-2-[rac-(2R,5S)-5-methyl-2-[2-[rac-(3S,4S)-1,3-dimethyl-4-piperidyl]indazol-6-yl]-1-piperidyl]acetamide (Compound 159)

To a solution of 2-oxo-2-[rac-(2R,5S)-5-methyl-2-[2-[rac-(3S,4S)-1,3-dimethyl-4-piperidyl]indazol-6-yl]-1-piperidyl]-N-[1-(2-trimethylsilylethoxymethyl)pyrazolo[4,3-c]pyridin-7-yl]acetamide (0.11 g, 85.29 μmol) in MeOH (1 mL) was added Hydrogen chloride solution 4.0M in dioxane (800.00 mg, 21.94 mmol, 1 mL) at 21° C. The resulting mixture was left to stir for 48 hr. The resulting mixture was evaporated to dryness and subjected to HPLC (30-50% 0.5-6.5 min; 30 ml/min water-acetonitrile+nh3 (loading pump 4 ml/min; acetonitrile); column xbridgeC18 19*100 mm (R)). 2-oxo-N-(1H-pyrazolo[4,3-c]pyridin-7-yl)-2-[rac-(2R,5S)-5-methyl-2-[2-[rac-(3S,4S)-1,3-dimethyl-4-piperidyl]indazol-6-yl]-1-piperidyl]acetamide (17.6 mg, 34.20 μmol, 40.10% yield) was obtained as a yellow solid.

¹H NMR (600 MHz, dmso) δ 0.50-0.55 (m, 3H), 0.79-1.09 (m, 3H), 1.29-1.45 (m, 1H), 1.65-1.82 (m, 2H), 1.86-1.97 (m, 2H), 2.00-2.17 (m, 2H), 2.17-2.21 (m, 1H), 2.22-2.25 (m, 3H), 2.27-2.35 (m, 2H), 2.88-2.93 (m, 2H), 3.37-3.45 (m, 1H), 3.73-4.16 (m, 2H), 5.48-5.83 (m, 1H), 6.98-7.11 (m, 1H), 7.55-7.61 (m, 1H), 7.63-7.76 (m, 1H), 8.28-8.42 (m, 2H), 8.42-8.59 (m, 1H), 8.87-9.00 (m, 1H), 10.97-11.27 (m, 1H), 12.82-13.19 (m, 1H).

LCMS(ESI): [M+H]⁺ m/z: calcd 514.4; found 515.2; Rt=2.025 min.

### Example 150. N-(4-amino-1H-pyrazolo[4,3-c]pyridin-7-yl)-2-(rac-(2S,5R)-2-(3,5-dichlorophenyl)-5-methylpiperidin-1-yl)-2-oxoacetamide (Compound 161)

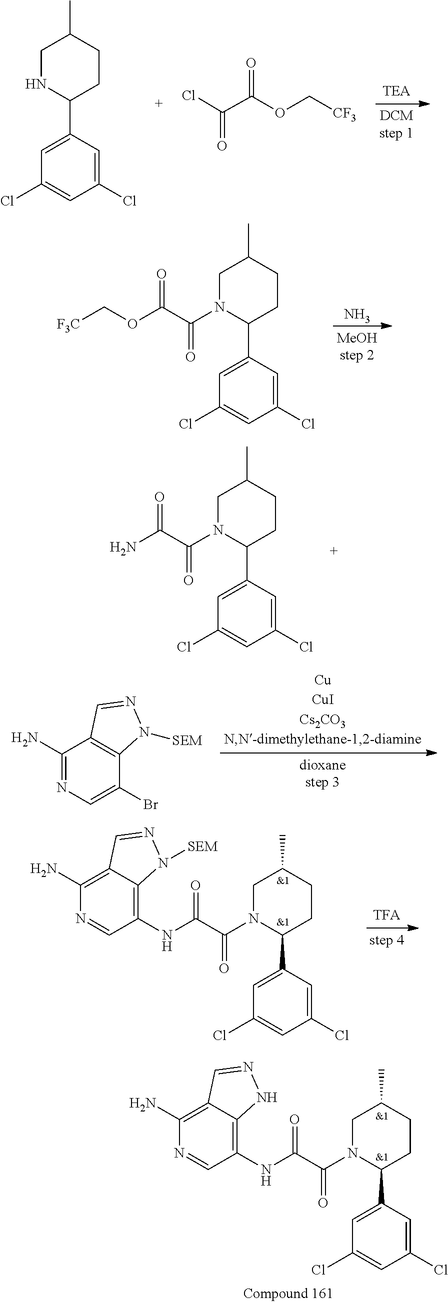

The synthesis of rac-(2R,5S)-2-(3,5-dichlorophenyl)-5-methylpiperidine is given by Intermediate 48.

### Step 1: Synthesis of 2,2,2-trifluoroethyl 2-(2-(3,5-dichlorophenyl)-5-methylpiperidin-1-yl)-2-oxoacetate

Prepared by general procedure scheme S9 step 1. Yield: 1 g of crude.

LCMS(ESI): [M]⁺ m/z: calcd 397.2; found 398.2; Rt=4.445 min.

### Step 2: Synthesis of 2-(2-(3,5-dichlorophenyl)-5-methylpiperidin-1-yl)-2-oxoacetamide

Gaseous ammonia (42.77 mg, 2.51 mmol) was passed through a solution of 2,2,2-trifluoroethyl 2-[2-(3,5-dichlorophenyl)-5-methyl-1-piperidyl]-2-oxo-acetate (1 g, 2.51 mmol) in MeOH (25 mL) and was stirred at 21° C. for 5 hr. The solvent was evaporated to obtain 2-[2-(3,5-dichlorophenyl)-5-methyl-1-piperidyl]-2-oxo-acetamide (0.74 g, 2.35 mmol, 93.49% yield) as a white solid.

LCMS(ESI): [M]⁺ m/z: calcd 315.2; found 316.2; Rt=3.673 min.

### Step 3: Synthesis of N-(4-amino-1-((2-(trimethylsilyl)ethoxy)methyl)-1H-pyrazolo[4,3-c]pyridin-7-yl)-2-(rac-(2S,5R)-2-(3,5-dichlorophenyl)-5-methylpiperidin-1-yl)-2-oxoacetamide

Prepared by general procedure scheme S9 step 3A. Yield: 13.6 mg (7.42%).

HPLC conditions: Column: XBridge C18 100*19 mm, 5 microM; 0.5-6.5 min 40-70% water-MeCN+NH₃ 30 ml/min; (loading pump 4 ml/min MeCN).

LCMS(ESI): [M]⁺ m/z: calcd 577.2; found 578.2; Rt=1.341 min.

### Step 4: Synthesis of N-(4-amino-1H-pyrazolo[4,3-c]pyridin-7-yl)-2-(rac-(2S,5R)-2-(3,5-dichlorophenyl)-5-methylpiperidin-1-yl)-2-oxoacetamide (Compound 161)

N-[4-Amino-1-(2-trimethylsilylethoxymethyl)pyrazolo[4,3-c]pyridin-7-yl]-2-oxo-2-[rac-(2S,5R)-2-(3,5-dichlorophenyl)-5-methyl-1-piperidyl]acetamide (0.017 g, 29.43 μmol) was dissolved in TFA (100.68 mg, 883.00 μmol, 68.03 μL) and stirred at 20° C. for 3 hr. The reaction mixture was submitted to HPLC (2-10 min 0-80% water/MeOH+NH₃ 30/min; loading pump 4 ml/min MeOH column SunFire 19*100 mm). N-(4-Amino-1H-pyrazolo[4,3-c]pyridin-7-yl)-2-oxo-2-[rac-(2S,5R)-2-(3,5-dichlorophenyl)-5-methyl-1-piperidyl]acetamide (0.0044 g, 9.84 μmol, 33.42% yield) was obtained as a light-yellow solid.

### Compound 161:

LCMS(ESI): [M]⁺ m/z: calcd 447.2; found 448.2; Rt=2.780 min.

### Example 151. 2-((2R,5S)-2-(2-(1,5-dimethylpiperidin-3-yl)benzo[d]thiazol-5-yl)-5-methylpiperidin-1-yl)-2-oxo-N-(1H-pyrazolo[4,3-c]pyridin-7-yl)acetamide (Compound 162, Compound 179 and Compound 171)

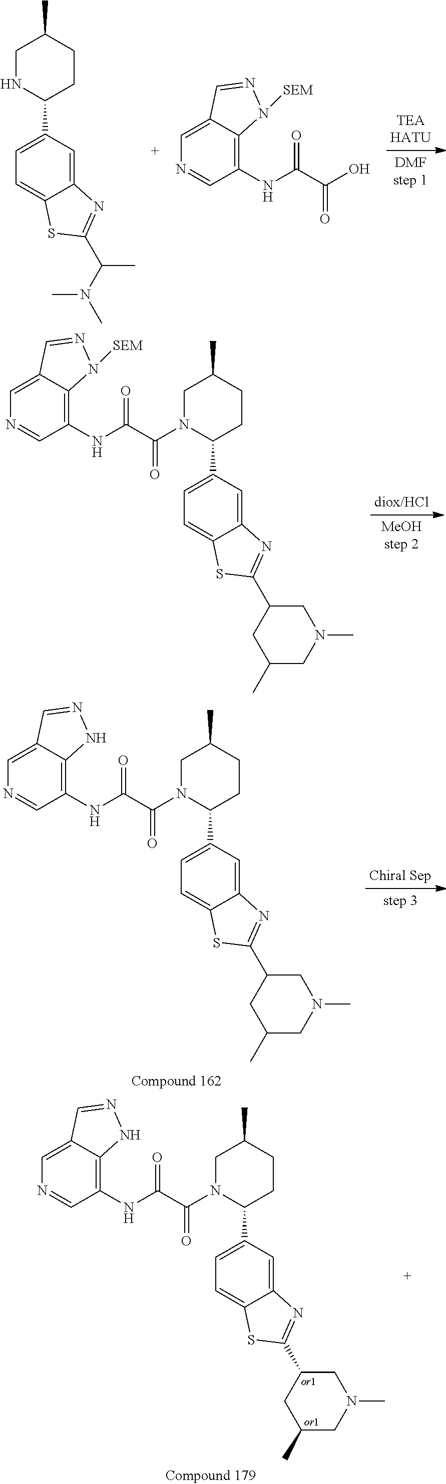

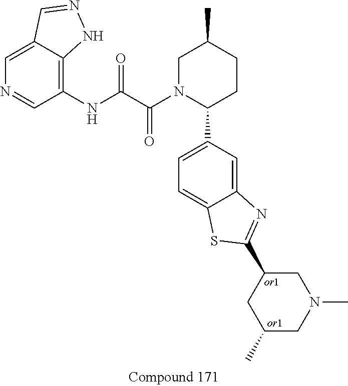 [SMILES: C[C@@H]1C[C@@H](c2nc3cc([C@H]4CC[C@H](C)CN4C(=O)C(=O)Nc4cncc5cn[nH]c45)ccc3s2)CN(C)C1]

Synthesis of 2-(1,5-dimethylpiperidin-3-yl)-5-((2R,5S)-5-methylpiperidin-2-yl)benzo[d]thiazole is given by Intermediate 49.

### Step 1: Synthesis of 2-((2R,5S)-2-(2-(1,5-dimethylpiperidin-3-yl)benzo[d]thiazol-5-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S4 step 6A. Yield: 160 mg (41.52%).

HPLC conditions: Column: XBridge C18 100*19 mm, 5 microM; 0-5 min 50-100% water-MeOH+0.1% NH₄OH, flow: 30 ml/min; (loading pump 4 ml/min MeOH).

LCMS(ESI): [M]⁺ m/z: calcd 661.2; found 662.2; Rt=1.221 min.

### Step 2: Synthesis of 2-((2R,5S)-2-(2-(1,5-dimethylpiperidin-3-yl)benzo[d]thiazol-5-yl)-5-methylpiperidin-1-yl)-2-oxo-N-(1H-pyrazolo[4,3-c]pyridin-7-yl)acetamide (Compound 162)

Hydrogen chloride solution 4.0M in dioxane (991.46 mg, 27.19 mmol, 1.24 mL) was added to a stirred solution of 2-oxo-2-[(2R,5S)-2-[2-(1,5-dimethyl-3-piperidyl)-1,3-benzothiazol-5-yl]-5-methyl-1-piperidyl]-N-[1-(2-trimethylsilylethoxymethyl)pyrazolo[4,3-c]pyridin-7-yl]acetamide (90.00 mg, 135.97 μmol) in MeOH (1.5 mL) and reaction mixture stirred at 20° C. for 24 hr. Volatiles was evaporated in vacuum, residue dissolved in methanol (8 mL), slowly quenched with TEA (0.5 g) and submitted to HPLC (column: XBridge C18 100×19 mm, 5 um; 50-100% 0-5 min H₂O/MeOH/0.1% NH₄OH, flow: 30 ml/min) to give pure 2-oxo-N-(1H-pyrazolo[4,3-c]pyridin-7-yl)-2-[(2R,5S)-2-[2-(1,5-dimethyl-3-piperidyl)-1,3-benzothiazol-5-yl]-5-methyl-1-piperidyl]acetamide (31 mg, 58.31 μmol, 42.88% yield) Compound 162: ¹H NMR (600 MHz, dmso) δ 0.89-1.11 (m, 7H), 1.19 (s, 1H), 1.33-1.43 (m, 1H), 1.56-1.96 (m, 5H), 2.00-2.35 (m, 8H), 2.84-2.90 (m, 1H), 3.75-4.16 (m, 1H), 5.52-5.84 (m, 1H), 7.33-7.51 (m, 1H), 7.89-7.94 (m, 1H), 8.02-8.11 (m, 1H), 8.28-8.40 (m, 1H), 8.40-8.63 (m, 1H), 8.81-9.11 (m, 1H), 11.03-11.23 (m, 1H), 12.91-13.18 (m, 1H).

LCMS(ESI): [M]⁺ m/z: calcd 531.2; found 532.2; Rt=1.903 min.

### Step 3: Chiral Separation (Compound 179 and Compound 171)

Racemic 2-oxo-N-(1H-pyrazolo[4,3-c]pyridin-7-yl)-2-[(2R,5S)-2-[2-(1,5-dimethyl-3-piperidyl)-1,3-benzothiazol-5-yl]-5-methyl-1-piperidyl]acetamide (31.00 mg, 58.31 μmol) was chiral separated (Column: Chiralpak IC II (250*20 mm, 5 mkm); Mobile phase: CO₂-MeOH, 60-40. Flow Rate: 10 mL/min) to obtain 2-oxo-N-(1H-pyrazolo[4,3-c]pyridin-7-yl)-2-[(2R,5S)-5-methyl-2-[2-[(3S,5S)-1,5-dimethyl-3-piperidyl]-1,3-benzothiazol-5-yl]-1-piperidyl]acetamide (6.1 mg, 11.47 μmol, 19.68% yield) and 2-oxo-N-(1H-pyrazolo[4,3-c]pyridin-7-yl)-2-[(2R,5S)-5-methyl-2-[2-[(3R,5R)-1,5-dimethyl-3-piperidyl]-1,3-benzothiazol-5-yl]-1-piperidyl]acetamide (6.8 mg, 12.79 μmol, 21.94% yield).

Rel Time for Compound 179 in analytical conditions (column: IC, CO₂-MeOH, 50-50, 2 ml/min as mobile phase) 15.61 min and for Compound 171 24.46 min.

Compound 179: Retention time: 15.61 min.

¹H NMR (600 MHz, DMSO-d₆) δ 0.86-0.93 (m, 3H), 1.03-1.10 (m, 3H), 1.12-1.18 (m, 1H), 1.19-1.27 (m, 4H), 1.49-1.55 (m, 1H), 1.73-2.02 (m, 4H), 2.07-2.19 (m, 2H), 2.21 (s, 3H), 2.77-2.82 (m, 1H), 3.15-3.19 (m, 1H), 3.77-4.15 (m, 1H), 5.54-5.81 (m, 1H), 7.38-7.47 (m, 1H), 7.88-7.94 (m, 1H), 8.01-8.14 (m, 1H), 8.29-8.39 (m, 1H), 8.39-8.58 (m, 1H), 8.88-8.99 (m, 1H), 11.05-11.24 (m, 1H), 12.97-13.13 (m, 1H).

LCMS(ESI): [M]⁺ m/z: calcd 531.2; found 532.2; Rt=3.001 min.

Compound 171: Retention time: 24.46 min.

¹H NMR (600 MHz, DMSO-d₆) δ 0.86-0.93 (m, 3H), 1.03-1.10 (m, 3H), 1.12-1.18 (m, 1H), 1.19-1.27 (m, 4H), 1.49-1.55 (m, 1H), 1.73-2.02 (m, 4H), 2.07-2.19 (m, 2H), 2.21 (s, 3H), 2.77-2.82 (m, 1H), 3.15-3.19 (m, 1H), 3.77-4.15 (m, 1H), 5.54-5.81 (m, 1H), 7.38-7.47 (m, 1H), 7.88-7.94 (m, 1H), 8.01-8.14 (m, 1H), 8.29-8.39 (m, 1H), 8.39-8.58 (m, 1H), 8.88-8.99 (m, 1H), 11.05-11.24 (m, 1H), 12.97-13.13 (m, 1H).

LCMS(ESI): [M]⁺ m/z: calcd 531.2; found 532.2; Rt=2.370 min.

### Example 152. 2-oxo-N-(1H-pyrazolo[4,3-c]pyridin-7-yl)-2-[(2R,5S)-2-[2-[2-(dimethylamino)ethyl]indazol-6-yl]-5-methyl-1-piperidyl]acetamide (Compound 163)

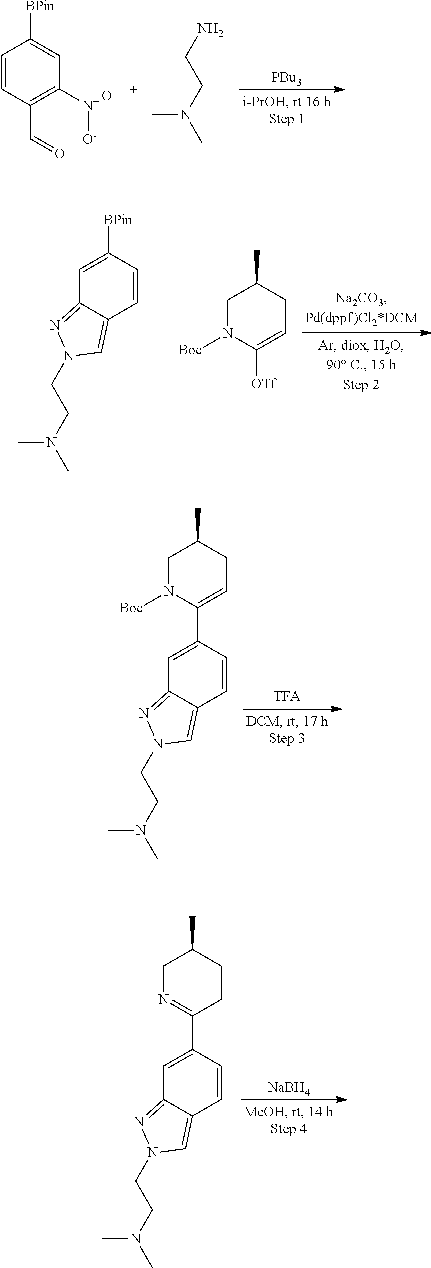

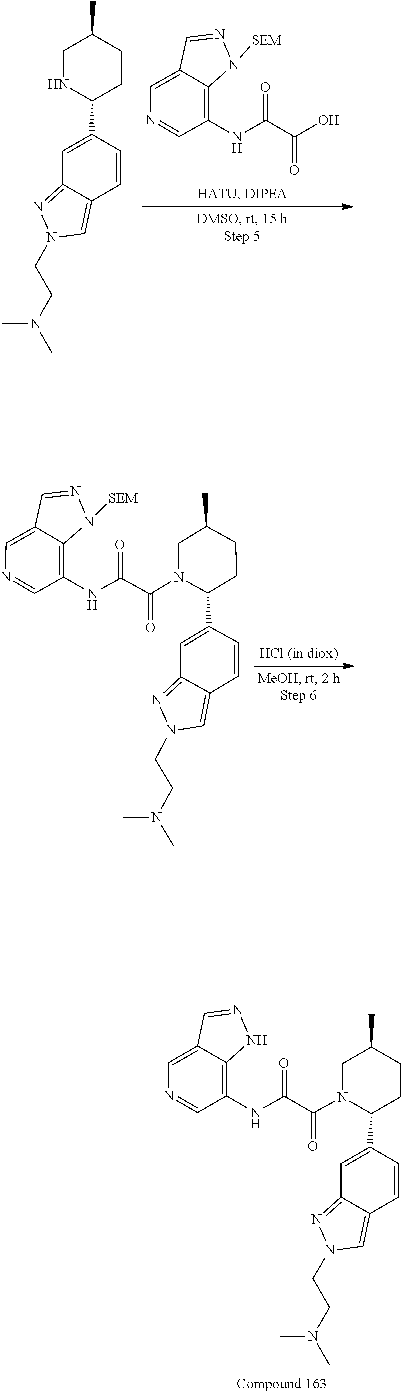

### Step 1: The Synthesis of N,N-dimethyl-2-[6-(4,4,5,5-tetramethyl-1,3,2-dioxaborolan-2-yl) indazol-2-yl]ethanamine

### Prepared by General Procedure Scheme S7 Step 1

The crude product was purified by FCC (MeOH in MTBE from 2% to 67%).

Yield: 1.45 g (50.98%)

LCMS(ESI): [M+H]⁺ m/z: calcd 315.2; found 316.2; Rt=1.056 min.

### Step 2: The Synthesis of tert-butyl (3S)-6-[2-[2-(dimethylamino)ethyl]indazol-6-yl]-3-methyl-3,4-dihydro-2H-pyridine-1-carboxylate

### Prepared by General Procedure Scheme S7 Step 2

Yield: 2.6 g (crude)

LCMS(ESI): [M+H]⁺ m/z: calcd 384.2; found 385.2; Rt=1.011 min.

### Step 3: The Synthesis of N,N-dimethyl-2-[6-[(3S)-3-methyl-2,3,4,5-tetrahydropyridin-6-yl]indazol-2-yl]ethanamine

### Prepared by General Procedure Scheme S7 Step 3

Yield: 0.9 g (46.8%)

LCMS(ESI): [M+H]⁺ m/z: calcd 284.2; found 285.2; Rt=0.407 min.

### Step 4: The Synthesis of N,N-dimethyl-2-[6-[(2R,5S)-5-methyl-2-piperidyl]indazol-2-yl]ethanamine

### Prepared by General Procedure Scheme S7 Step 4 (Method B)

Yield: 0.7 g (77.2%)

LCMS(ESI): [M+H]⁺ m/z: calcd 286.2; found 287.2; Rt=0.414 min.

### Step 5: The Synthesis of 2-oxo-2-[(2R,5S)-2-[2-[2-(dimethylamino)ethyl]indazol-6-yl]-5-methyl-1-piperidyl]-N-[1-(2-trimethylsilylethoxymethyl)pyrazolo[4,3-c]pyridin-7-yl]acetamide

### Prepared by General Procedure Scheme S7 Step 5

HPLC conditions: 2-10 min 30-60% methanol+FA, 30 ml/min (loading pump 4 ml methanol+FA) column: YMC-ACTUS TRIART C18 100*20 5 microM Yield: 45 mg (8.7%)

LCMS(ESI): [M+H]⁺ m/z: calcd 604.4; found 605.4; Rt=1.022 min.

### Step 6: Synthesis of 2-oxo-N-(1H-pyrazolo[4,3-c]pyridin-7-yl)-2-[(2R,5S)-2-[2-[2-(dimethylamino)ethyl]indazol-6-yl]-5-methyl-1-piperidyl]acetamide (Compound 163)

2-oxo-2-[(2R,5S)-2-[2-[2-(dimethylamino)ethyl]indazol-6-yl]-5-methyl-1-piperidyl]-N-[1-(2-trimethylsilylethoxymethyl)pyrazolo[4,3-c]pyridin-7-yl]acetamide (0.045 g, 60.57 μmol, 3HCOOH) was dissolved in Trifluoroacetic acid, 99% (207.21 mg, 1.82 mmol, 140.00 μL) and stirred at 20° C. for 3 hr. The reaction mixture was submitted to HPLC (2-10 min 30-60% methanol+NH₃ 30/min; loading pump 4 ml/min methanol+NH₃ column SunFire 19*100 mm. 2-oxo-N-(1H-pyrazolo[4,3-c]pyridin-7-yl)-2-[(2R,5S)-2-[2-[2-(dimethylamino)ethyl]indazol-6-yl]-5-methyl-1-piperidyl]acetamide (0.015 g, 31.61 μmol, 52.18% yield) was obtained as a light-yellow solid.

¹H NMR (600 MHz, dmso) δ 0.75-1.08 (m, 3H), 1.31-1.47 (m, 1H), 1.66-1.84 (m, 1H), 1.87-1.99 (m, 1H), 2.06-2.15 (m, 1H), 2.15-2.19 (m, 6H), 2.25-2.35 (m, 1H), 2.72-2.78 (m, 2H), 2.86-2.90 (m, 0.3H), 3.35-3.38 (m, 0.7H), 3.72-4.16 (m, 1H), 4.44-4.54 (m, 2H), 5.51-5.82 (m, 1H), 6.99-7.14 (m, 1H), 7.54 (s, 1H), 7.65-7.78 (m, 1H), 8.25-8.41 (m, 2H), 8.43-8.60 (m, 1H), 8.87-9.04 (m, 1H), 10.97-11.23 (m, 1H), 12.84-13.29 (m, 1H).

LCMS(ESI): [M+H]⁺ m/z: calcd 474.3; found 475.2; Rt=1.525 min.

### Example 153. N-(4-amino-1H-pyrazolo[4,3-c]pyridin-7-yl)-2-((2R,5S)-2-(3-((S)-2-(dimethylamino)propoxy)phenyl)-5-methylpiperidin-1-yl)-2-oxoacetamide (Compound 164)

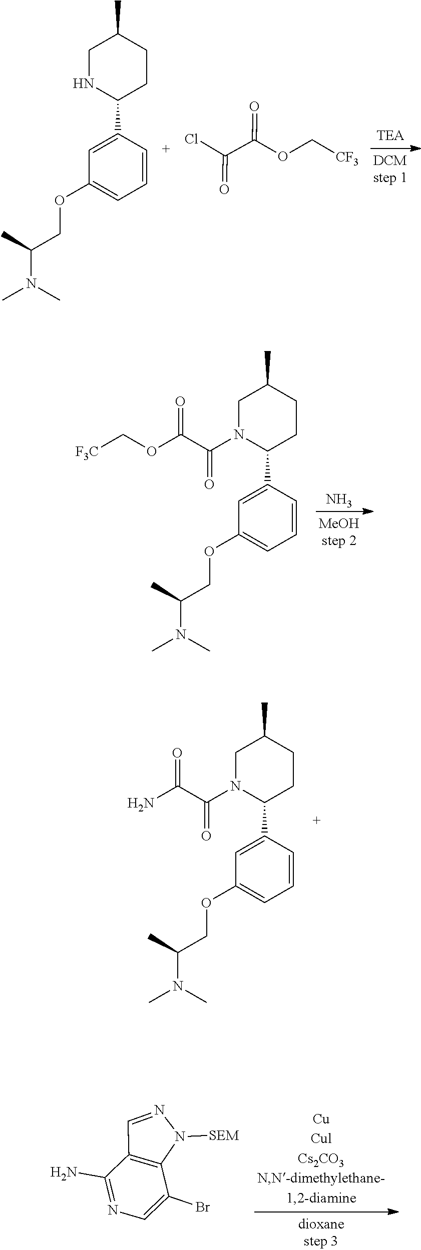

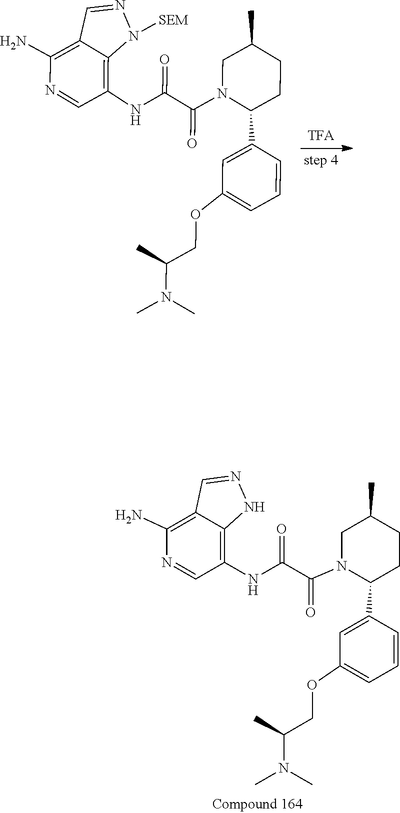 [SMILES: C[C@H]1CC[C@H](c2cccc(OC[C@H](C)N(C)C)c2)N(C(=O)C(=O)Nc2cnc(N)c3cnn(COCC[Si](C)(C)C)c23)C1 ; C[C@H]1CC[C@H](c2cccc(OC[C@H](C)N(C)C)c2)N(C(=O)C(=O)Nc2cnc(N)c3cn[nH]c23)C1]

The Synthesis of (S)—N,N-dimethyl-1-(3-((2R,5S)-5-methylpiperidin-2-yl)phenoxy)propan-2-amine is given by Intermediate 44.

### Step 1: Synthesis of 2,2,2-trifluoroethyl 2-((2R,5S)-2-(3-((S)-2-(dimethylamino)propoxy)phenyl)-5-methylpiperidin-1-yl)-2-oxoacetate

Prepared by general procedure scheme S9 step 1. Yield: 590 mg (96.90%).

LCMS(ESI): [M]⁺ m/z: calcd 430.2; found 431.2; Rt=1.099 min.

### Step 2: Synthesis of 2-((2R,5S)-2-(3-((S)-2-(dimethylamino)propoxy)phenyl)-5-methylpiperidin-1-yl)-2-oxoacetamide

2,2,2-Trifluoroethyl 2-oxo-2-[(2R,5S)-5-methyl-2-[3-[(2S)-2-(dimethylamino)propoxy]phenyl]-1-piperidyl]acetate (590 mg, 1.37 mmol) was dissolved in NH₃/MeOH (30 mL) and the resulting mixture was stirred overnight. The reaction mixture was concentrated in vacuum to obtain 2-oxo-2-[(2R,5S)-5-methyl-2-[3-[(2S)-2-(dimethylamino)propoxy]phenyl]-1-piperidyl]acetamide (457 mg, 1.32 mmol, 95.96% yield).

LCMS(ESI): [M]⁺ m/z: calcd 347.2; found 348.2; Rt=0.851 min.

### Step 3: Synthesis of N-(4-amino-1-((2-(trimethylsilyl)ethoxy)methyl)-1H-pyrazolo[4,3-c]pyridin-7-yl)-2-((2R,5S)-2-(3-((S)-2-(dimethylamino)propoxy)phenyl)-5-methylpiperidin-1-yl)-2-oxoacetamide

Prepared by general procedure scheme S9 step 3A. Yield: 30.1 mg of crude.

HPLC conditions: Column: SunFire C18 100*19 mm, 5 microM; 2-10 min 30-80% MeOH+FA 30 ml/min; (loading pump 4 ml/min MeOH).

LCMS(ESI): [M]⁺ m/z: calcd 609.2; found 610.2; Rt=0.895 min.

### Step 4: Synthesis of N-(4-amino-1H-pyrazolo[4,3-c]pyridin-7-yl)-2-((2R,5S)-2-(3-((S)-2-(dimethylamino)propoxy)phenyl)-5-methylpiperidin-1-yl)-2-oxoacetamide (Compound 164)

N-[4-Amino-1-(2-trimethylsilylethoxymethyl)pyrazolo[4,3-c]pyridin-7-yl]-2-oxo-2-[(2R,5S)-5-methyl-2-[3-[(2S)-2-(dimethylamino)propoxy]phenyl]-1-piperidyl]acetamide (30.1 mg, 49.36 μmol) was dissolved in TFA (1 mL) and stirred overnight. The reaction mixture was concentrated in vacuum and the residue was purified by HPLC (0-80% MeOH+NH₃ 30 ml/min) to obtain N-(4-amino-1H-pyrazolo[4,3-c]pyridin-7-yl)-2-oxo-2-[(2R,5S)-5-methyl-2-[3-[(2S)-2-(dimethylamino)propoxy]phenyl]-1-piperidyl]acetamide (4.7 mg, 9.80 μmol, 19.86% yield).

Compound 164: LCMS(ESI): [M]⁺ m/z: calcd 479.2; found 480.2; Rt=1.945 min.

### Example 154. N-(4-amino-1H-pyrazolo[4,3-c]pyridin-7-yl)-2-((2R,5S)-2-(3-(2-(dimethylamino)ethoxy)phenyl)-5-methylpiperidin-1-yl)-2-oxoacetamide (Compound 166)

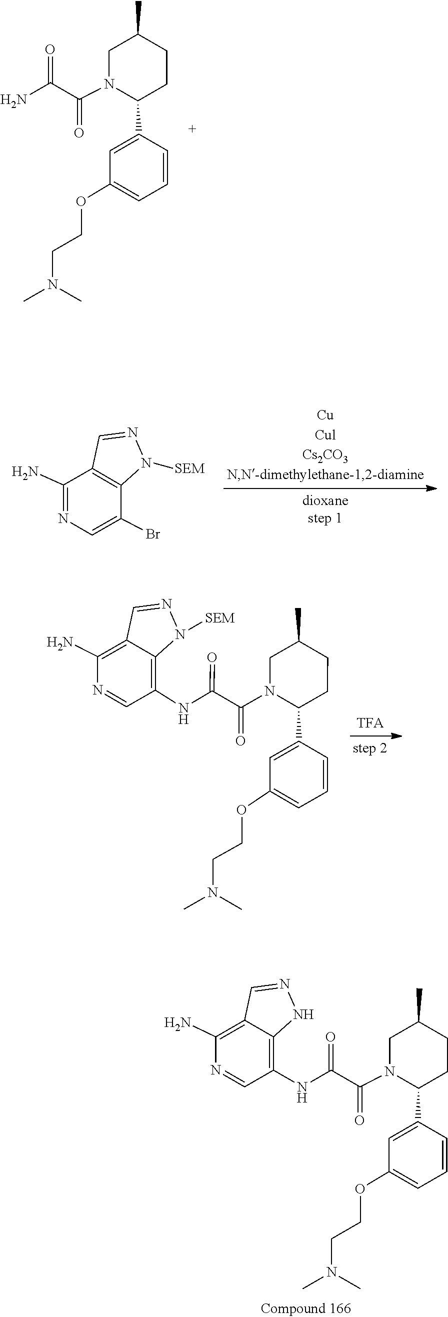 [SMILES: C[C@H]1CC[C@H](c2cccc(OCCN(C)C)c2)N(C(=O)C(N)=O)C1 ; C[Si](C)(C)CCOCn1ncc2c(N)ncc(Br)c21 ; C[C@H]1CC[C@H](c2cccc(OCCN(C)C)c2)N(C(=O)C(=O)Nc2cnc(N)c3cnn(COCC[Si](C)(C)C)c23)C1 ; C[C@H]1CC[C@H](c2cccc(OCCN(C)C)c2)N(C(=O)C(=O)Nc2cnc(N)c3cn[nH]c23)C1]

The synthesis of 2-((2R,5S)-2-(3-(2-(dimethylamino)ethoxy)phenyl)-5-methylpiperidin-1-yl)-2-oxoacetamide is by Intermediate 50.

### Step 1: Synthesis of N-(4-amino-1-((2-(trimethylsilyl)ethoxy)methyl)-1H-pyrazolo[4,3-c]pyridin-7-yl)-2-((2R,5S)-2-(3-(2-(dimethylamino)ethoxy)phenyl)-5-methylpiperidin-1-yl)-2-oxoacetamide

Prepared by general procedure scheme S9 step 3A. Yield: 26.5 mg (9.89%).

HPLC conditions: Column: XBridge C18 100*19 mm, 5 microM; 0-5 min 0-50% MeCN+FA 30 ml/min; (loading pump 4 ml/min MeCN).

LCMS(ESI): [M]⁺ m/z: calcd 595.2; found 596.2; Rt=0.955 min.

### Step 2: Synthesis of N-(4-amino-1H-pyrazolo[4,3-c]pyridin-7-yl)-2-((2R,5S)-2-(3-(2-(dimethylamino)ethoxy)phenyl)-5-methylpiperidin-1-yl)-2-oxoacetamide (Compound 166)

N-[4-Amino-1-(2-trimethylsilylethoxymethyl)pyrazolo[4,3-c]pyridin-7-yl]-2-oxo-2-[(2R,5S)-2-[3-[2-(dimethylamino)ethoxy]phenyl]-5-methyl-1-piperidyl]acetamide (37.9 mg, 63.61 μmol) was dissolved in TFA (1 mL) and the resulting mixture was stirred overnight. The reaction mixture was concentrated in vacuum and the residue was purified by HPLC (2-10 min 10-50% MeOH+NH₃, 30 ml/min) to obtain N-(4-amino-1H-pyrazolo[4,3-c]pyridin-7-yl)-2-oxo-2-[(2R,5S)-2-[3-[2-(dimethylamino)ethoxy]phenyl]-5-methyl-1-piperidyl]acetamide (4.4 mg, 9.45 μmol, 14.86% yield) and N-(4-amino-1H-pyrazolo[4,3-c]pyridin-7-yl)-2-oxo-2-[(2R,5S)-2-[3-[2-(dimethylamino)ethoxy]phenyl]-5-methyl-1-piperidyl]acetamide (3 mg, 6.44 μmol, 10.13% yield).

Compound 166: ¹H NMR (600 MHZ, DMSO-d₆) δ (ppm) 1.03 (m, 3H), 1.35 (m, 1H), 1.70 (m, 2H), 1.88 (m, 1H), 2.03 (m, 1H), 2.25 (s, 7H), 2.67 (m, 2H), 4.03 (m, 3H), 5.56 (m, 1H), 6.66 (m, 2H), 6.86 (m, 2H), 6.91 (m, 1H), 7.30 (m, 1H), 7.69 (m, 1H), 8.18 (m, 1H), 10.49 (m, 1H), 12.76 (m, 1H).

LCMS(ESI): [M]⁺ m/z: calcd 465.2; found 466.2; Rt=1.875 min.

### Example 155. N-(imidazo[1,2-a]pyridin-7-yl)-2-((2R,5S)-5-methyl-2-(2-(1-methylpiperidin-4-yl)quinolin-7-yl)piperidin-1-yl)-2-oxoacetamide (Compound 170)

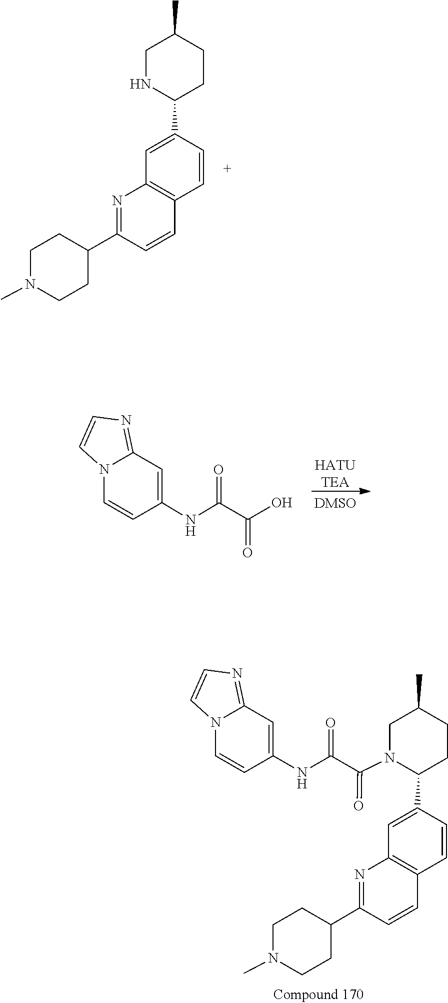 [SMILES: C[C@H]1CC[C@H](c2ccc3ccc(C4CCN(C)CC4)nc3c2)NC1 ; O=C(O)C(=O)Nc1ccn2ccnc2c1 ; "HATUTEA" ; C[C@H]1CC[C@H](c2ccc3ccc(C4CCN(C)CC4)nc3c2)N(C(=O)C(=O)Nc2ccn3ccnc3c2)C1]

The synthesis of 2-(1-methyl-4-piperidyl)-7-[(2R,5S)-5-methyl-2-piperidyl]quinoline is given by Intermediate 22.

Crude product 2-(1-methyl-4-piperidyl)-7-[(2R,5S)-5-methyl-2-piperidyl]quinoline (100.00 mg, 216.40 μmol) from previous stage was mixed with TEA (109.49 mg, 1.08 mmol, 150.81 μL) in DMSO (2 mL), next 2-(imidazo[1,2-a]pyridin-7-ylamino)-2-oxo-acetic acid (66.60 mg, 324.60 μmol) and HATU (123.42 mg, 324.60 μmol) were added and stirred overnight. RM was treated with water and desired product was filtered, washed with water and dissolved in MeOH, then subjected to HPLC (2-10 min 10-40% MeOH+NH₃ 30 ml/min (loading pump 4 m MeOH+NH₃) column: SunFire 100*19 mm, 5 microM). N-Imidazo[1,2-a]pyridin-7-yl-2-oxo-2-[(2R,5S)-5-methyl-2-[2-(1-methyl-4-piperidyl)-7-quinolyl]-1-piperidyl]acetamide (15.7 mg, 30.75 μmol, 14.21% yield) was obtained.

Compound 170: ¹H NMR (600 MHZ, DMSO-d₆) δ (ppm) 1.06 (dd, 3H), 1.39 (t, 1H), 1.73 (m, 1H), 1.89 (m, 5H), 2.08 (m, 3H), 2.23 (m, 3H), 2.35 (m, 1H), 2.81 (m, 1H), 2.93 (m, 2H), 3.91 (m, 2H), 5.57 (m, 1H), 7.06 (m, 1H), 7.47 (m, 3H), 7.90 (m, 4H), 8.24 (m, 1H), 8.46 (m, 1H), 11.17 (m, 1H).

LCMS(ESI): [M]⁺ m/z: calcd 510.2; found 511.2; Rt=1.858 min.

### Example 156. 2-oxo-N-(1H-pyrazolo[4,3-c]pyridin-7-yl)-2-[rac-(2R,5S)-5-methyl-2-[2-[rac-(3R,4S)-1,3-dimethyl-4-piperidyl]indazol-6-yl]-1-piperidyl]acetamide (Compound 173)

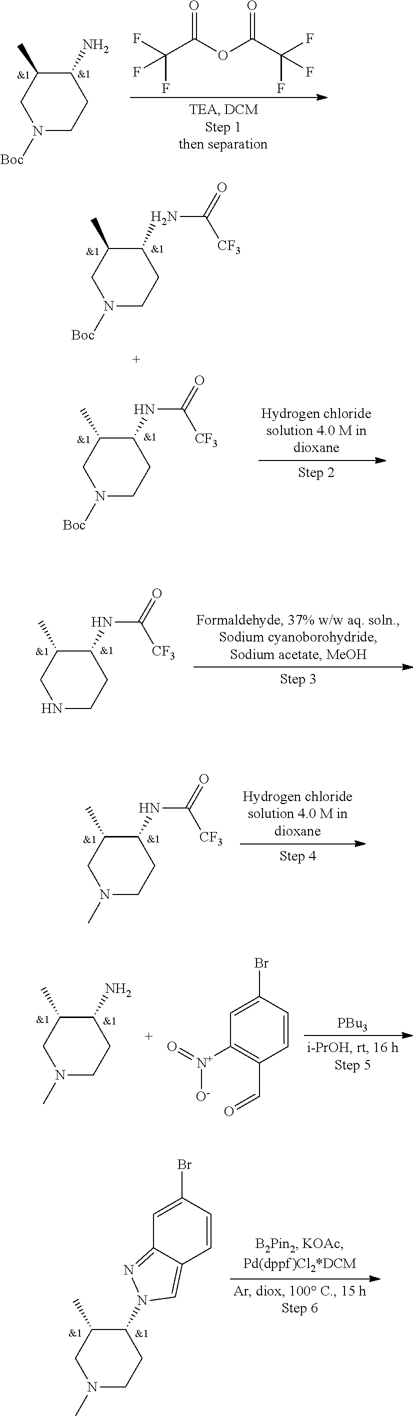

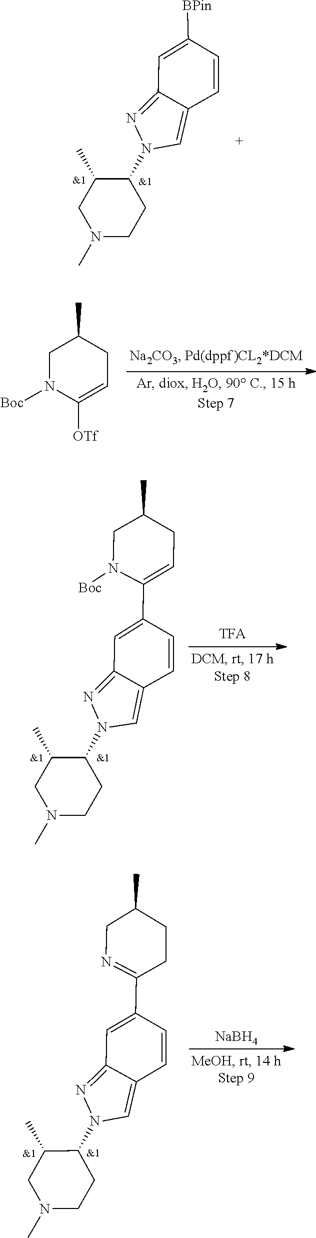

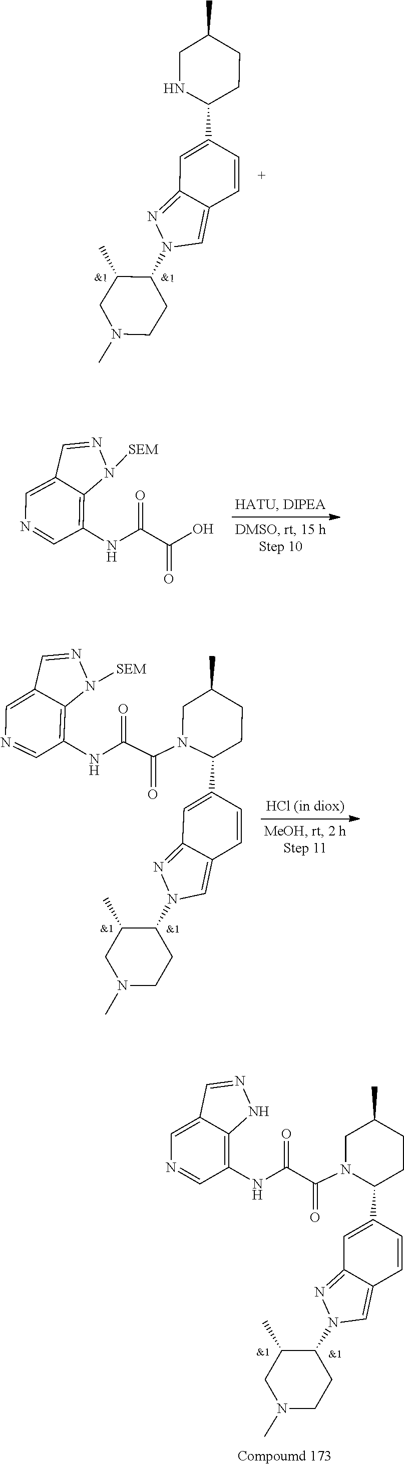 [SMILES: C[C@H]1CC[C@H](c2ccc3cn([C@@H]4CCN(C)C[C@@H]4C)nc3c2)NC1 ; C[Si](C)(C)CCOCn1ncc2cncc(NC(=O)C(=O)O)c21 ; C[C@H]1CC[C@H](c2ccc3cn([C@@H]4CCN(C)C[C@@H]4C)nc3c2)N(C(=O)C(=O)Nc2cncc3cnn(COCC[Si](C)(C)C)c23)C1 ; C[C@H]1CC[C@H](c2ccc3cn([C@@H]4CCN(C)C[C@@H]4C)nc3c2)N(C(=O)C(=O)Nc2cncc3cn[nH]c23)C1]

### Step 1 is Given by Example 149, Step 1

### Step 2: The Synthesis of 2,2,2-trifluoro-N-[rac-(3S,4R)-3-methyl-4-piperidyl]acetamide

To a solution of tert-butyl rac-(3S,4R)-3-methyl-4-[(2,2,2-trifluoroacetyl)amino]piperidine-1-carboxylate (7.8 g, 25.14 mmol) in Et2O was added Hydrogen chloride solution 4.0M in dioxane (38.05 g, 1.04 mol, 47.56 mL) at 21° C. The resulting mixture was left to stir for 48 hr. The resulting mixture was evaporated to dryness to give 2,2,2-trifluoro-N-[rac-(3S,4R)-3-methyl-4-piperidyl]acetamide (6 g, 24.33 mmol, 96.77% yield, HCl) as a white solid.

¹H NMR (400 MHz, dmso) δ 0.85 (d, 3H), 1.83-1.91 (m, 2H), 2.22 (m, 1H), 3.00-3.10 (m, 4H), 4.16 (m, 1H), 9.17-9.36 (brs, 3H).

### Step 3: The Synthesis of 2,2,2-trifluoro-N-[rac-(3S,4R)-1,3-dimethyl-4-piperidyl]acetamide

Formaldehyde, 37% w/w aq. soln., stab. with 7-8% methanol (2.37 g, 29.19 mmol, 2.19 mL, 37% purity) and acetic acid (4.38 g, 72.98 mmol, 4.18 mL) were added to the solution of 2,2,2-trifluoro-N-[rac-(3S,4R)-3-methyl-4-piperidyl]acetamide (6 g, 24.33 mmol, HCl) and Sodium acetate, anhydrous (2.19 g, 26.76 mmol, 1.44 mL) in MeOH (45.56 mL). Resulting mixture was stirred at 21° C. for 1 hour before Sodium cyanoborohydride (3.06 g, 48.65 mmol) was added thereto. After that, stirring was continued for 21 hr. Then, the solvent was removed under reduced pressure, and residue was partitioned between sat. aq. NaHCO₃ solution (50 ml) and EtOAc (50 mL). Water was back-extracted with EtOAc (3×50 mL). The organics were combined and evaporated to give 2,2,2-trifluoro-N-[rac-(3S,4R)-1,3-dimethyl-4-piperidyl]acetamide (4.7 g, 20.96 mmol, 86.17% yield) as a white solid.

¹H NMR (400 MHZ, CDCl₃) δ 0.983 (d, 3H), 1.77-1.82 (m, 4H), 2.14 (m, 2H), 2.27 (s, 3H), 2.46 (m, 1H), 4.11 (m, 1H), 6.22 (brs, 1H).

### Step 4: The Synthesis of rac-(3S,4R)-1,3-dimethylpiperidin-4-amine

2,2,2-trifluoro-N-[rac-(3S,4R)-1,3-dimethyl-4-piperidyl]acetamide (4.7 g, 20.96 mmol) was dissolved in MeOH (25 mL) and Hydrogen chloride solution 36% in water (764.27 mg, 20.96 mmol, 25 mL) was added thereto. The resulting mixture was stirred at 80° C. for 48 hr. The reaction mixture was evaporated to dryness to give rac-(3S,4R)-1,3-dimethylpiperidin-4-amine (4.75 g, crude, 2HCl) as a white solid. The solid rac-(3S,4R)-1,3-dimethylpiperidin-4-amine (4.75 g, 23.62 mmol, 99%) was used in the next step without further purification.

LCMS(ESI): [M+H]⁺ m/z: calcd 128.1; found 129.0; Rt=0.238 min.

### Step 5: The Synthesis of 6-bromo-2-[rac-(3S,4R)-1,3-dimethyl-4-piperidyl]indazole

### Prepared by General Procedure Scheme S7 Step 1A

Yield: 4.1 g (63.25%)

¹H NMR (500 MHz, DMSO-d₆) δ 0.62 (d, 3H), 1.98 (d, 1H), 2.06 (t, 1H), 2.17 (s, 3H), 2.16 (m, 1H), 2.20 (s, 3H), 2.17 (d, 1H), 2.24 (m, 2H), 2.68 (d, 1H), 2.94 (d, 1H), 4.56 (m, 1H), 7.12 (d, 1H), 7.68 (d, 1H), 7.85 (s, 1H), 8.42 (s, 1H).

### Step 6: The Synthesis of 2-[rac-(3S,4S)-1,3-dimethyl-4-piperidyl]-6-(4,4,5,5-tetramethyl-1,3,2-dioxaborolan-2-yl)indazole

### Prepared by General Procedure Scheme S7 Step 2A

Yield: 8 g (crude)

LCMS(ESI): [M+H]⁺ m/z: calcd 355.2; found 356.4; Rt=3.053 min.

### Step 7: The Synthesis of tert-butyl rac-(3S)-3-methyl-6-[2-[rac-(3S,4R)-1,3-dimethyl-4-piperidyl]indazol-6-yl]-3,4-dihydro-2H-pyridine-1-carboxylate

### Prepared by General Procedure Scheme S7 Step 2

Yield: 12.0 g (crude)

LCMS(ESI): [M+H]⁺ m/z: calcd 424.3; found 425.4; Rt=2.916 min.

### Step 8: The Synthesis of 2-[rac-(3S,4R)-1,3-dimethyl-4-piperidyl]-6-[rac-(3S)-3-methyl-2,3,4,5-tetrahydropyridin-6-yl]indazole

### Prepared by General Procedure Scheme S7 Step 3 (Method A)

Yield: 6.8 g (crude)

LCMS(ESI): [M+H]⁺ m/z: calcd 324.2; found 325.2; Rt=1.55 min.

### Step 9: The Synthesis of 2-[rac-(3S,4R)-1,3-dimethyl-4-piperidyl]-6-[rac-(2R,5S)-5-methyl-2-piperidyl]indazole

### Prepared by General Procedure Scheme S7 Step 4

Yield: 1 g (29.23%)

LCMS(ESI): [M+H]⁺ m/z: calcd 326.2; found 327.2; Rt=0.719 min.

### Step 10: The Synthesis of 2-oxo-2-[rac-(2R,5S)-5-methyl-2-[2-[rac-(3R,4S)-1,3-dimethyl-4-piperidyl]indazol-6-yl]-1-piperidyl]-N-[1-(2-trimethylsilylethoxymethyl)pyrazolo[4,3-c]pyridin-7-yl]acetamide

### Prepared by General Procedure Scheme S7 Step 5

Yield: 120 mg (crude)

LCMS(ESI): [M+H]⁺ m/z: calcd 644.4; found 645.2; Rt=3.26 min.

### Step 11: The Synthesis of 2-oxo-N-(1H-pyrazolo[4,3-c]pyridin-7-yl)-2-[rac-(2R,5S)-5-methyl-2-[2-[rac-(3R,4S)-1,3-dimethyl-4-piperidyl]indazol-6-yl]-1-piperidyl]acetamide (Compound 173)

To a solution of 2-oxo-2-[rac-(2R,5S)-5-methyl-2-[2-[rac-(3R,4S)-1,3-dimethyl-4-piperidyl]indazol-6-yl]-1-piperidyl]-N-[1-(2-trimethylsilylethoxymethyl)pyrazolo[4,3-c]pyridin-7-yl]acetamide (120 mg, 93.04 μmol) in MeOH (1 mL) was added Hydrogen chloride solution 4.0M in dioxane (800.00 mg, 21.94 mmol, 1 mL) at 21° C. The resulting mixture was left to stir for 48 hr. The resulting mixture was evaporated to dryness and subjected to HPLC (30-50% 0.5-6.5 min; 30 ml/min water-acetonitrile+nh3 (loading pump 4 ml/min; acetonitrile); column xbridgeC18 19*100 mm (R)). 2-oxo-N-(1H-pyrazolo[4,3-c]pyridin-7-yl)-2-[rac-(2R,5S)-5-methyl-2-[2-[rac-(3R,4S)-1,3-dimethyl-4-piperidyl]indazol-6-yl]-1-piperidyl]acetamide (25.7 mg, 49.94 μmol, 53.68% yield) was obtained as a yellow solid.

¹H NMR (600 MHz, dmso) δ 0.58-0.69 (m, 3H), 0.99-1.12 (m, 3H), 1.32-1.45 (m, 1H), 1.75-1.86 (m, 1H), 1.88-2.01 (m, 2H), 2.02-2.16 (m, 2H), 2.20 (s, 3H), 2.25-2.33 (m, 2H), 2.39-2.45 (m, 2H), 2.67-2.76 (m, 1H), 2.84-3.22 (m, 2H), 3.73-4.16 (m, 1H), 4.50-4.64 (m, 1H), 5.47-5.82 (m, 1H), 6.96-7.12 (m, 1H), 7.52-7.61 (m, 1H), 7.64-7.76 (m, 1H), 8.23-8.40 (m, 2H), 8.41-8.60 (m, 1H), 8.83-9.01 (m, 1H), 11.01-11.21 (m, 1H), 12.92-13.24 (m, 1H).

LCMS(ESI): [M+H]⁺ m/z: calcd 514.4; found 515.2; Rt=2.042 min.

### Example 157. N-(7-amino-1H-pyrazolo[3,4-c]pyridin-4-yl)-2-((2R,5S)-2-(benzo[d]thiazol-5-yl)-5-methylpiperidin-1-yl)-2-oxoacetamide (Compound 174)

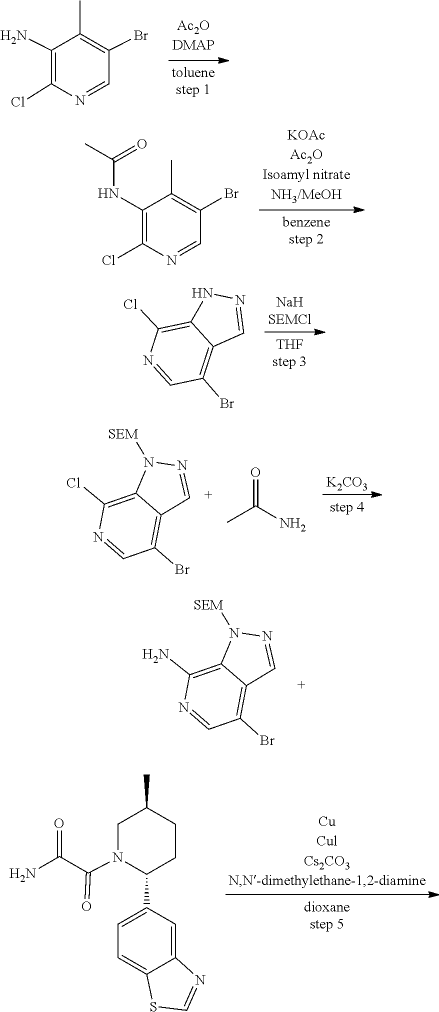

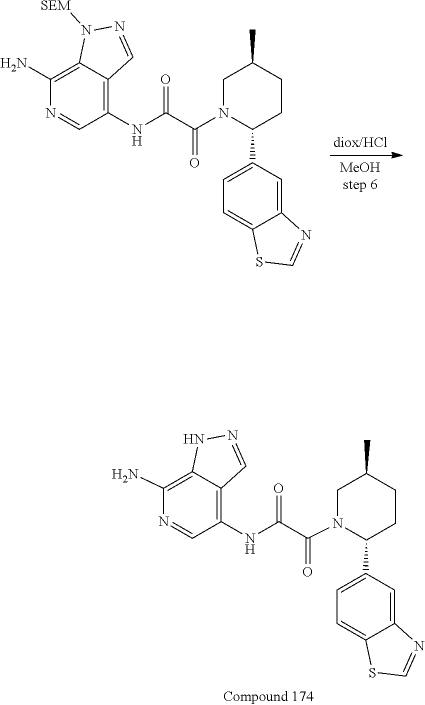 [SMILES: C[C@H]1CC[C@H](c2ccc3scnc3c2)N(C(=O)C(=O)Nc2cnc(N)c3c2cnn3COCC[Si](C)(C)C)C1 ; C[C@H]1CC[C@H](c2ccc3scnc3c2)N(C(=O)C(=O)Nc2cnc(N)c3[nH]ncc23)C1]

### Step 1: Synthesis of N-(5-bromo-2-chloro-4-methylpyridin-3-yl)acetamide

5-Bromo-2-chloro-4-methyl-pyridin-3-amine (8 g, 36.12 mmol), DMAP (4.41 g, 36.12 mmol) and acetic anhydride (5.90 g, 57.79 mmol, 5.46 mL) were mixed in toluene (100 mL) and heated for 12 hr at 100° C. Reaction mixture was concentrated in vacuum and subjected to column flash silica gel chromatography (eluent gradient chloroform-acetonitrile). N-(5-Bromo-2-chloro-4-methyl-3-pyridyl)acetamide (6.3 g, 23.91 mmol, 66.19% yield) was obtained.

LCMS(ESI): [M]⁺ m/z: calcd 263.2; found 264.2; Rt=0.948 min.

### Step 2: Synthesis of 4-bromo-7-chloro-1H-pyrazolo[3,4-c]pyridine

Potassium acetate (3.52 g, 35.86 mmol, 2.24 mL) and acetic anhydride (7.32 g, 71.72 mmol, 6.77 mL) were added to a solution of the N-(5-bromo-2-chloro-4-methyl-3-pyridyl)acetamide (6.3 g, 23.91 mmol) in dry benzene (200 mL). The reaction mixture was heated at reflux, isoamyl nitrite (5.60 g, 47.81 mmol, 6.40 mL) was added at this temperature and reflux was continued for 16 hr. The insoluble material was then filtered off and the filtrate was vacuum evaporated. The residue was dissolved in a saturated solution of ammonium in MeOH (407.15 mg, 23.91 mmol) and the resulting solution was stirred at rt for 2 hr and then concentrated in vacuum. Crude product was dissolved in 200 ml of EtOAc and washed with water. Organic phase was dried over Na₂SO₄ and concentrated in vacuum to give 4-bromo-7-chloro-1H-pyrazolo[3,4-c]pyridine (4.3 g, crude).

LCMS(ESI): [M]⁺ m/z: calcd 232.2; found 233.2; Rt=1.018 min.

### Step 3: Synthesis of 4-bromo-7-chloro-1-((2-(trimethylsilyl)ethoxy)methyl)-1H-pyrazolo[3,4-c]pyridine

Sodium hydride (in oil dispersion) 60% dispersion in mineral oil (0.9 g, 22.50 mmol) was added portion wise to the solution of 4-bromo-7-chloro-1H-pyrazolo[3,4-c]pyridine (4.3 g, 14.80 mmol) in dry THF (50 mL). After 30 min the reaction mixture was cooled to 5° C. with ice-water bath and 2-(chloromethoxy)ethyl-trimethyl-silane (3.21 g, 19.24 mmol, 3.40 mL) in 10 ml THF was added dropwise, then stirred overnight at rt. The reaction mixture was diluted with water and desired compound was extracted with DCM (2*100 ml), dried over Na₂SO₄ and concentrated in vacuum to give 2-[(4-bromo-7-chloro-pyrazolo[3,4-c]pyridin-1-yl)methoxy]ethyl-trimethyl-silane (3.5 g, crude).

LCMS(ESI): [M]⁺ m/z: calcd 362.2; found 363.2; Rt=1.749 min.

### Step 4: Synthesis of 4-bromo-1-((2-(trimethylsilyl)ethoxy)methyl)-1H-pyrazolo[3,4-c]pyridin-7-amine

Mixture of 2-[(4-bromo-7-chloro-pyrazolo[3,4-c]pyridin-1-yl)methoxy]ethyl-trimethyl-silane (2 g, 4.14 mmol), acetamide (3.66 g, 62.03 mmol) and potassium carbonate, anhydrous, 99% (2.86 g, 20.68 mmol, 1.25 mL) was stirred at 210° C. for 5 hr. After cooling to rt, the reaction mixture was poured into water and desired product was extracted with DCM, dried over sodium sulfate and evaporated. Part of crude material was subjected to HPLC (Device (Mobile Phase, Column): 2-10 min 30-65% MeCN+FA, 30 ml/min). 4-Bromo-1-(2-trimethylsilylethoxymethyl)pyrazolo[3,4-c]pyridin-7-amine (94 mg, crude) was obtained.

LCMS(ESI): [M]⁺ m/z: calcd 343.2; found 344.2; Rt=3.022 min.

### Step 5: Synthesis of N-(7-amino-1-((2-(trimethylsilyl)ethoxy)methyl)-1H-pyrazolo[3,4-c]pyridin-4-yl)-2-((2R,5S)-2-(benzo[d]thiazol-5-yl)-5-methylpiperidin-1-yl)-2-oxoacetamide

Prepared by general procedure scheme S3 step 3A. Yield: 8.8 mg of crude.

HPLC conditions: Column: SunFire C18 100*19 mm, 5 microM; 2-10 min 0-45% MeCN+FA, flow: 30 ml/min; (loading pump 4 ml/min MeCN).

LCMS(ESI): [M]⁺ m/z: calcd 565.2; found 566.2; Rt=1.045 min.

### Step 6: Synthesis of N-(7-amino-1H-pyrazolo[3,4-c]pyridin-4-yl)-2-((2R,5S)-2-(benzo[d]thiazol-5-yl)-5-methylpiperidin-1-yl)-2-oxoacetamide (Compound 174)

N-[7-Amino-1-(2-trimethylsilylethoxymethyl)pyrazolo[3,4-c]pyridin-4-yl]-2-oxo-2-[(2R,5S)-2-(1,3-benzothiazol-5-yl)-5-methyl-1-piperidyl]acetamide (15.3 mg, 27.04 μmol) was dissolved in mixture of MeOH (1 mL) and dioxane (3 mL) saturated with HCl (10% by weight) HCl (986.02 μg, 27.04 μmol) and stirred for 3 hr, then concentrated in vacuum and subjected to HPLC (Device (Mobile Phase, Column): 2-10 min 30-60 MeOH+FA 30 ml/min). N-(7-Amino-1H-pyrazolo[3,4-c]pyridin-4-yl)-2-oxo-2-[(2R,5S)-2-(1,3-benzothiazol-5-yl)-5-methyl-1-piperidyl]acetamide (3.9 mg, 8.10 μmol, 29.95% yield, HCOOH) was obtained.

Compound 174: LCMS(ESI): [M]⁺ m/z: calcd 435.2; found 436.2; Rt=2.046 min.

### Example 158. N-(4-amino-1H-pyrazolo[4,3-c]pyridin-7-yl)-2-((2R,5S)-5-methyl-2-(4-((1-methylpiperidin-4-yl)oxy)phenyl)piperidin-1-yl)-2-oxoacetamide (Compound 175)

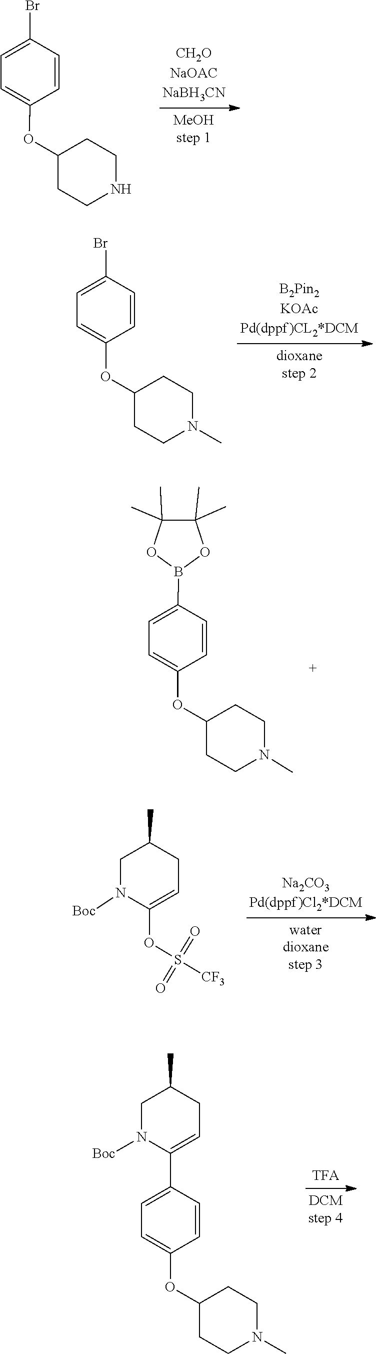

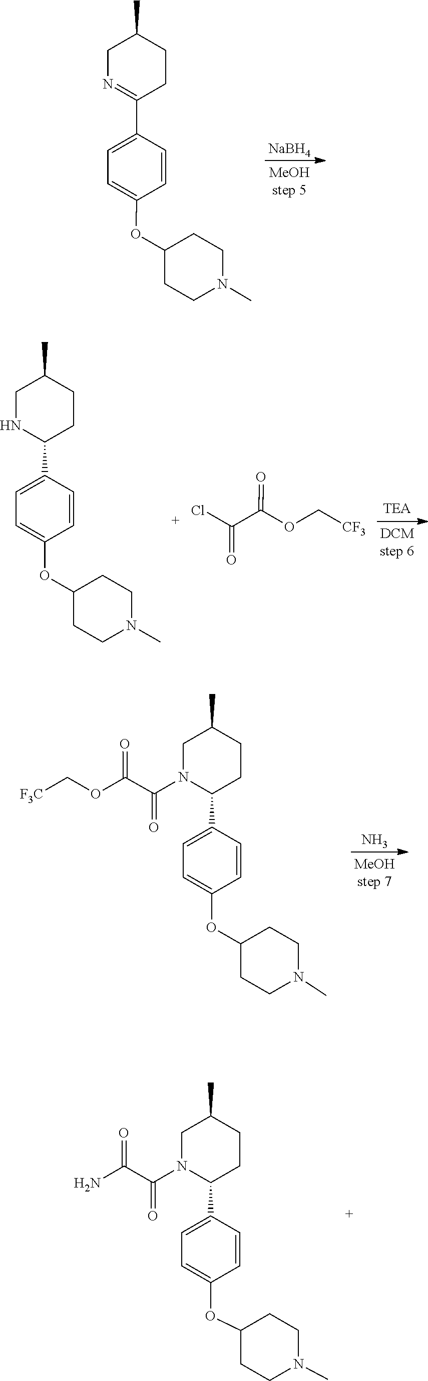

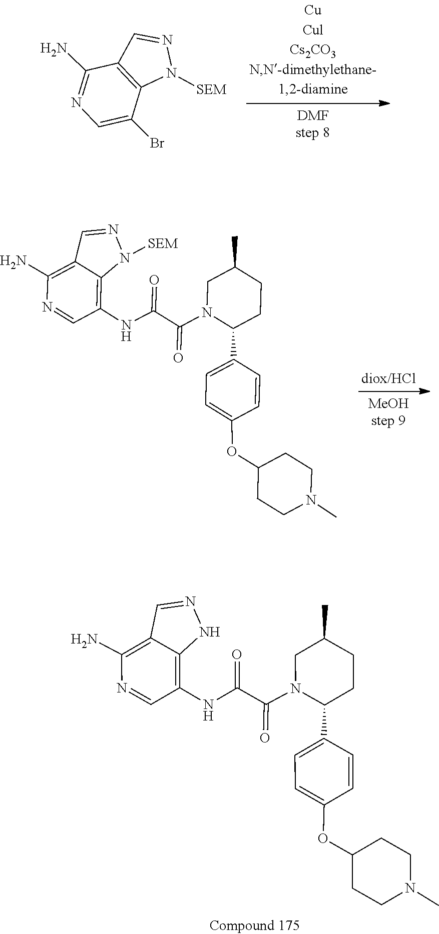 [SMILES: C[Si](C)(C)CCOCn1ncc2c(N)ncc(Br)c21 ; C[C@H]1CC[C@H](c2ccc(OC3CCN(C)CC3)cc2)N(C(=O)C(=O)Nc2cnc(N)c3cnn(COCC[Si](C)(C)C)c23)C1 ; C[C@H]1CC[C@H](c2ccc(OC3CCN(C)CC3)cc2)N(C(=O)C(=O)Nc2cnc(N)c3cn[nH]c23)C1]

### Step 1: Synthesis of 4-(4-bromophenoxy)-1-methylpiperidine

Formaldehyde, 37% w/w aq. soln., stab. with 7-8% MeOH (468.90 mg, 15.62 mmol, 432.97 μL) and sodium acetate (1.28 g, 15.62 mmol, 838.41 μL) were added to the solution of 4-(4-bromophenoxy)piperidine (2 g, 7.81 mmol) in MeOH (28.73 mL). Resulting mixture was stirred at 25° C. for 1 hr before sodium cyan borohydride (981.38 mg, 15.62 mmol) was added thereto. After that, stirring was continued for 16 hr. Then, solvent was removed under reduced pressure and residue was partitioned between 15% aq. K₂CO₃ solution (30 ml) and DCM (50 ml). Organic layer was separated, dried over solid Na₂SO₄ and concentrated under reduced pressure, leaving 4-(4-bromophenoxy)-1-methyl-piperidine (2 g, 7.40 mmol, 94.81% yield).

LCMS(ESI): [M]⁺ m/z: calcd 270.2; found 271.2; Rt=0.814 min.

### Step 2: Synthesis of 1-methyl-4-(4-(4,4,5,5-tetramethyl-1,3,2-dioxaborolan-2-yl)phenoxy)piperidine

Prepared by general procedure scheme S8 step 1. Yield: 2.3 g of crude.

LCMS(ESI): [M]⁺ m/z: calcd 317.2; found 318.2; Rt=0.971 min.

### Step 3: Synthesis of (S)-tert-butyl 3-methyl-6-(4-((1-methylpiperidin-4-yl)oxy)phenyl)-3,4-dihydropyridine-1(2H)-carboxylate

Prepared by general procedure scheme S8 step 2. Yield: 2.8 g of crude.

LCMS(ESI): [M]⁺ m/z: calcd 386.2; found 387.2; Rt=1.149 min.

### Step 4: Synthesis of (S)-3-methyl-6-(4-((1-methylpiperidin-4-yl)oxy)phenyl)-2,3,4,5-tetrahydropyridine

Prepared by general procedure scheme S8 step 3. Yield: 1.4 g of crude.

LCMS(ESI): [M]⁺ m/z: calcd 286.2; found 287.2; Rt=0.608 min.

### Step 5: Synthesis of 1-methyl-4-(4-((2R,5S)-5-methylpiperidin-2-yl)phenoxy)piperidine

Prepared by general procedure scheme S8 step 4. Yield: 1 g of crude.

LCMS(ESI): [M]⁺ m/z: calcd 288.2; found 289.2; Rt=0.709 min.

### Step 6: Synthesis of 2,2,2-trifluoroethyl 2-((2R,5S)-5-methyl-2-(4-((1-methylpiperidin-4-yl)oxy)phenyl)piperidin-1-yl)-2-oxoacetate

Prepared by general procedure scheme S9 step 1. Yield: 0.45 g of crude.

LCMS(ESI): [M]⁺ m/z: calcd 442.2; found 443.2; Rt=1.138 min.

### Step 7: Synthesis of 2-((2R,5S)-5-methyl-2-(4-((1-methylpiperidin-4-yl)oxy)phenyl)piperidin-1-yl)-2-oxoacetamide

Prepared by general procedure scheme S9 step 2. Yield: 0.3 g of crude.

LCMS(ESI): [M]⁺ m/z: calcd 359.2; found 360.2; Rt=0.746 min.

### Step 8: Synthesis of N-(4-amino-1-((2-(trimethylsilyl)ethoxy)methyl)-1H-pyrazolo[4,3-c]pyridin-7-yl)-2-((2R,5S)-5-methyl-2-(4-((1-methylpiperidin-4-yl)oxy)phenyl)piperidin-1-yl)-2-oxoacetamide

Prepared by general procedure scheme S9 step 3A. Yield: 80 mg (15.41%).

HPLC conditions: Column: SunFire C18 100*19 mm, 5 microM; 2-10 min 0-50% MeCN+FA 30 ml/min; (loading pump 4 ml/min MeCN).

LCMS(ESI): [M]⁺ m/z: calcd 621.2; found 622.2; Rt=0.954 min.

### Step 8: Synthesis of N-(4-amino-1H-pyrazolo[4,3-c]pyridin-7-yl)-2-((2R,5S)-5-methyl-2-(4-((1-methylpiperidin-4-yl)oxy)phenyl)piperidin-1-yl)-2-oxoacetamide (Compound 175)

N-[4-Amino-1-(2-trimethylsilylethoxymethyl)pyrazolo[4,3-c]pyridin-7-yl]-2-oxo-2-[(2R,5S)-5-methyl-2-[4-[(1-methyl-4-piperidyl)oxy]phenyl]-1-piperidyl]acetamide (80 mg, 128.65 μmol) was dissolved in MeOH (5 mL) and diox/HCl (128.65 μmol, 2 mL) was added. Then mixture was stirred at rt 16 hr. The solution was evaporated and submitted to reverse phase HPLC (column: SunFire C18 100×19 mm, 5 um; mobile phase: 30-60% 2-10 min MeCN+FA, flow rate: 30 ml/min) to afford N-(4-amino-1H-pyrazolo[4,3-c]pyridin-7-yl)-2-oxo-2-[(2R,5S)-5-methyl-2-[4-[(1-methyl-4-piperidyl)oxy]phenyl]-1-piperidyl]acetamide (8.8 mg, 16.37 μmol, 12.72% yield, HCOOH).

Compound 175: ¹H NMR (600 MHz, DMSO-d₆) δ (ppm) 0.60-1.11 (m, 3H), 1.11-1.53 (m, 1H), 1.54-1.77 (m, 3H), 1.77-1.98 (m, 3H), 1.98-2.16 (m, 1H), 2.16-2.24 (m, 5H), 2.61-2.76 (m, 2H), 3.04-3.14 (m, 2H), 3.73-4.05 (m, 1H), 4.23-4.43 (m, 1H), 5.22-6.42 (m, 1H), 6.57-6.87 (m, 2H), 6.86-7.20 (m, 2H), 7.19-7.76 (m, 2H), 8.11-8.22 (m, 2H), 9.52-10.64 (m, 1H), 12.44-13.51 (m, 1H).

LCMS(ESI): [M]⁺ m/z: calcd 491.2; found 492.2; Rt=1.915 min.

### Example 159. 2-((2R,5S)-2-(3-cyano-2-(1-methylpiperidin-4-yl)quinolin-7-yl)-5-methylpiperidin-1-yl)-2-oxo-N-(1H-pyrazolo[4,3-c]pyridin-7-yl)acetamide (Compound 176)

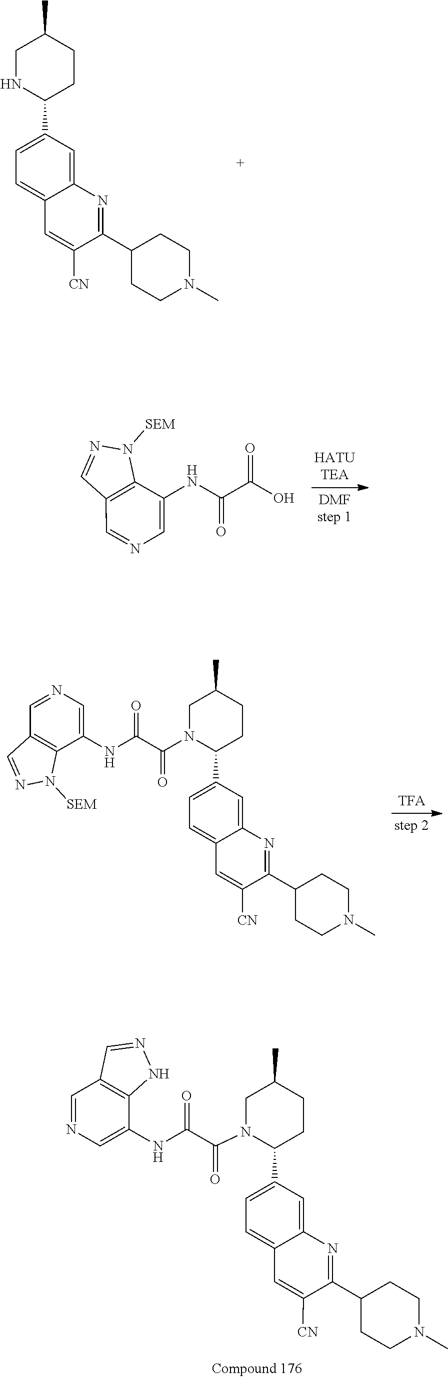 [SMILES: C[C@H]1CC[C@H](c2ccc3cc(C#N)c(C4CCN(C)CC4)nc3c2)NC1 ; C[Si](C)(C)CCOCn1ncc2cncc(NC(=O)C(=O)O)c21 ; C[C@H]1CC[C@H](c2ccc3cc(C#N)c(C4CCN(C)CC4)nc3c2)N(C(=O)C(=O)Nc2cncc3cnn(COCC[Si](C)(C)C)c23)C1 ; C[C@H]1CC[C@H](c2ccc3cc(C#N)c(C4CCN(C)CC4)nc3c2)N(C(=O)C(=O)Nc2cncc3cn[nH]c23)C1]

The synthesis of 7-((2R,5S)-5-methylpiperidin-2-yl)-2-(1-methylpiperidin-4-yl)quinoline-3-carbonitrile is given by Intermediate 51.

### Step 1: Synthesis of 2-((2R,5S)-2-(3-cyano-2-(1-methylpiperidin-4-yl)quinolin-7-yl)-5-methylpiperidin-1-yl)-2-oxo-N-(1-((2-(trimethylsilyl)ethoxy)methyl)-1H-pyrazolo[4,3-c]pyridin-7-yl)acetamide

Prepared by general procedure scheme S8 step 5A. Yield: 200 mg (41.80%).

HPLC conditions: Column: XBridge BEH C18 100*19 mm, 5 microM; 0-1-6 min 40-40-65% water-MeCN+0.1% NH₄OH, flow: 30 ml/min; (loading pump 4 ml/min MeOH).

LCMS(ESI): [M]⁺ m/z: calcd 666.2; found 667.2; Rt=1.113 min.

### Step 2: Synthesis of 2-((2R,5S)-2-(3-cyano-2-(1-methylpiperidin-4-yl)quinolin-7-yl)-5-methylpiperidin-1-yl)-2-oxo-N-(1H-pyrazolo[4,3-c]pyridin-7-yl)acetamide (Compound 176)

2-Oxo-2-[(2R,5S)-2-[3-cyano-2-(1-methyl-4-piperidyl)-7-quinolyl]-5-methyl-1-piperidyl]-N-[1-(2-trimethylsilylethoxymethyl)pyrazolo[4,3-c]pyridin-7-yl]acetamide (150 mg, 224.93 μmol) solution in was treated with TFA (1.48 g, 12.98 mmol, 1 mL). The resulting mixture was stirred at 25° C. for 13 hr. The reaction mixture was concentrated under reduce pressure. The residue was purified by reverse phase HPLC (SYSTEM 30-80% 0-5 min H₂O/MeOH/0.1% NH₄OH, flow: 30 ml/min (loading pump 4 ml/min MeCN) target mass 536.64 column: XBridge C18 100×19 mm, 5 um) to give 2-oxo-N-(1H-pyrazolo[4,3-c]pyridin-7-yl)-2-[(2R,5S)-2-[3-cyano-2-(1-methyl-4-piperidyl)-7-quinolyl]-5-methyl-1-piperidyl]acetamide (110 mg, 204.98 μmol, 91.13% yield).

Compound 176: ¹H NMR (600 MHZ, DMSO-d₆) δ (ppm) 1.05-1.10 (m, 3H), 1.37-1.48 (m, 1H), 1.64-1.78 (m, 1H), 1.89-2.01 (m, 1H), 2.07-2.24 (m, 5H), 2.27-2.36 (m, 1H), 2.68-2.80 (m, 3H), 2.96-3.12 (m, 2H), 3.40-3.56 (m, 4H), 3.82-4.23 (m, 1H), 5.63-5.91 (m, 1H), 7.72-7.79 (m, 1H), 7.86-7.94 (m, 1H), 8.03-8.17 (m, 1H), 8.22-8.39 (m, 1H), 8.38-8.56 (m, 1H), 8.85-8.99 (m, 1H), 8.99-9.12 (m, 1H), 10.88-11.34 (m, 1H), 12.78-13.23 (m, 1H).

LCMS(ESI): [M]⁺ m/z: calcd 536.2; found 537.2; Rt=1.930 min.

### Example 160. N-(4-amino-1H-pyrazolo[4,3-c]pyridin-7-yl)-2-oxo-2-[(2R,5S)-5-methyl-2-(2-methylindazol-6-yl)-1-piperidyl]acetamide (Compound 180)

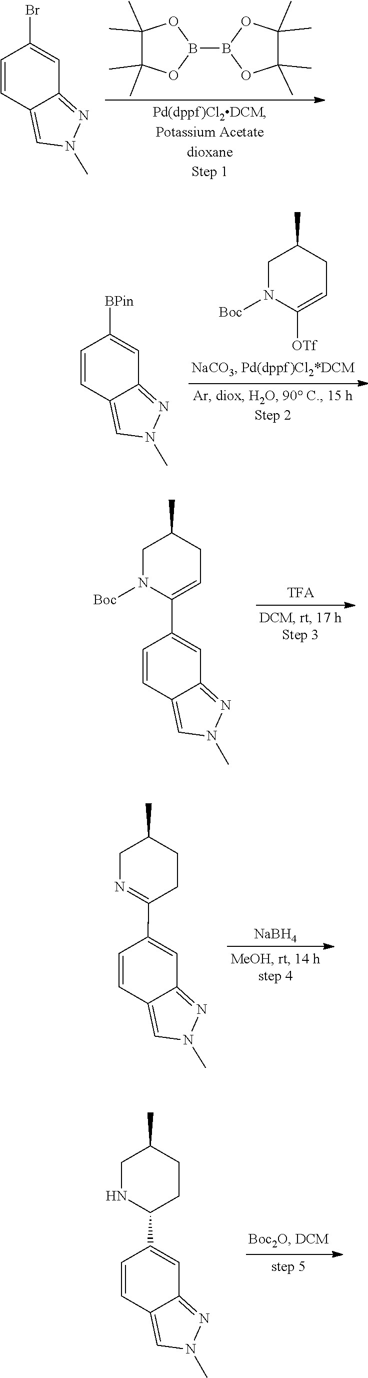

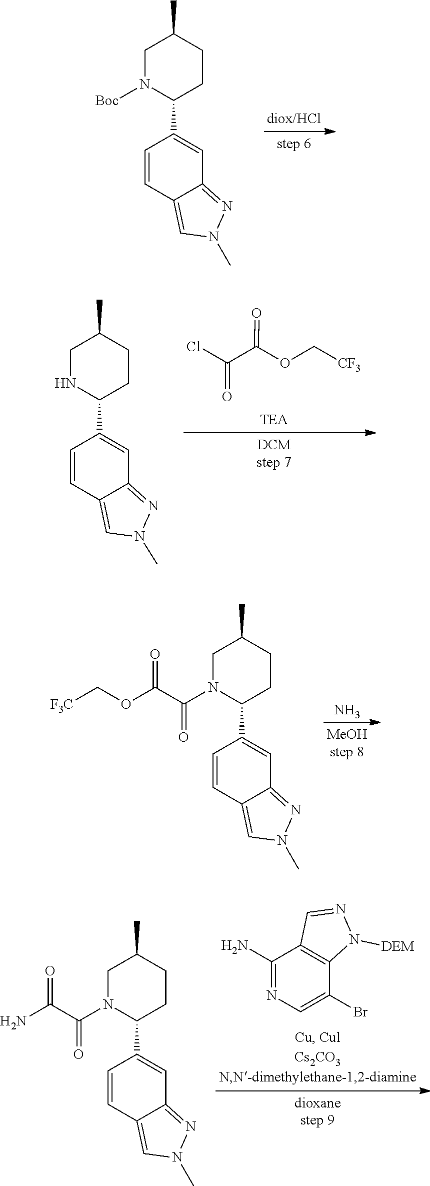

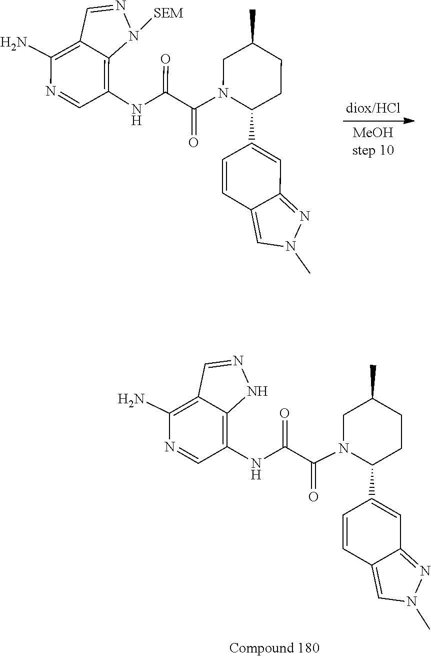 [SMILES: C[C@H]1CC[C@H](c2ccc3cn(C)nc3c2)N(C(=O)C(=O)Nc2cnc(N)c3cnn(COCC[Si](C)(C)C)c23)C1 ; C[C@H]1CC[C@H](c2ccc3cn(C)nc3c2)N(C(=O)C(=O)Nc2cnc(N)c3cn[nH]c23)C1]

The sample of Compound 180 was synthesized by the same route without steps 5-6. For 2^{nd} lot, steps 5-6 were performed to remove cis impurity earlier.

### Step 1: The Synthesis of 2-methyl-6-(4,4,5,5-tetramethyl-1,3,2-dioxaborolan-2-yl)indazole

A solution of 6-bromo-2-methyl-indazole (20 g, 94.76 mmol), 4,4,5,5-tetramethyl-2-(4,4,5,5-tetramethyl-1,3,2-dioxaborolan-2-yl)-1,3,2-dioxaborolane (24.06 g, 94.76 mmol), Pd(dppf)Cl2·DCM (3.87 g, 4.74 mmol, 2.46 mL) and Potassium Acetate (13.95 g, 142.14 mmol, 8.89 mL) in Dioxane (350 mL) was heated at 90° C. for 48 hr. An aliquot showed full conversion of SM. Reaction mixture was filtered and directly used for the next step without further treatment.

LCMS(ESI): [M+H]⁺ m/z: calcd 258.1; found 259.2; Rt=1.332 min.

### Step 2: The Synthesis of tert-butyl (3S)-3-methyl-6-(2-methylindazol-6-yl)-3,4-dihydro-2H-pyridine-1-carboxylate

To a solution of 2-methyl-6-(4,4,5,5-tetramethyl-1,3,2-dioxaborolan-2-yl)indazole (24 g, 92.98 mmol), tert-butyl (3S)-3-methyl-6-(trifluoromethylsulfonyloxy)-3,4-dihydro-2H-pyridine-1-carboxylate (32.11 g, 92.98 mmol), Sodium carbonate (29.56 g, 278.94 mmol, 11.68 mL) in Dioxane (350 mL) and Water (150 mL), Pd(dppf)Cl2·DCM (3.80 g, 4.65 mmol) was added under Argon atmosphere. The resulting mixture was stirred at 100° C. for 24 hr and cooled. The organic layer was separated, evaporated, the residue was dissolved in MTBE (500 ml), dried over Na2SO4, filtered and evaporated in vacuo to give tert-butyl (3S)-3-methyl-6-(2-methylindazol-6-yl)-3,4-dihydro-2H-pyridine-1-carboxylate (30 g, 91.63 mmol, 98.54% yield). Crude substance was used for the next step without further purification.

LCMS(ESI): [M+H]⁺ m/z: calcd 327.2; found 328.2; Rt=1.393 min.

### Step 3: The Synthesis of 2-methyl-6-[(3S)-3-methyl-2,3,4,5-tetrahydropyridin-6-yl]indazole

A solution of tert-butyl (3S)-3-methyl-6-(2-methylindazol-6-yl)-3,4-dihydro-2H-pyridine-1-carboxylate (30 g, 91.63 mmol) in Trifluoroacetic acid (74.00 g, 648.99 mmol, 50 mL) was stirred at 25° C. for 2 hr. The solvent was removed in vacuo, the residue was diluted with water (200 ml), pH was adjusted to 9-10 with NaOH solution, extracted with DCM (3*100 ml), dried over Na2SO4 and evaporated to give 2-methyl-6-[(3S)-3-methyl-2,3,4,5-tetrahydropyridin-6-yl]indazole (18 g, 79.19 mmol, 86.43% yield). This compound was used for the next step without further purification.

LCMS(ESI): [M+H]⁺ m/z: calcd 227.3; found 228.4; Rt=0.727 min.

### Step 4: The Synthesis of 2-methyl-6-[(2R,5S)-5-methyl-2-piperidyl]indazole

Sodium Borohydride (3.00 g, 79.19 mmol, 2.79 mL) was added portionwise to a stirred solution of 2-methyl-6-[(3S)-3-methyl-2,3,4,5-tetrahydropyridin-6-yl]indazole (18 g, 79.19 mmol) in Methanol (250 mL) at 0° C. The resulting mixture was stirred at 0° C. for 3 hr, and then evaporated in vacuo. The residue was diluted with water (100 ml) and extracted with dichloromethane (3*80 ml). The combined organic extracts were dried over sodium sulphate and evaporated in vacuo to afford 2-methyl-6-[(2R,5S)-5-methyl-2-piperidyl]indazole (15 g, 65.41 mmol, 82.60% yield).

LCMS(ESI): [M+H]⁺ m/z: calcd 229.2; found 230.2; Rt=0.772 min.

### Step 5: The Synthesis of tert-butyl (2R,5S)-5-methyl-2-(2-methylindazol-6-yl)piperidine-1-carboxylate

To a solution of 2-methyl-6-[(2R,5S)-5-methyl-2-piperidyl]indazole (15 g, 65.41 mmol) in DCM (200 mL), Di-tert-butyl dicarbonate (14.28 g, 65.41 mmol, 15.01 mL) was added dropwise. The resulting mixture was stirred at 25° C. for 3 hr evaporated and the residue was purified by gradient chromatography (hexane-ethylacetate) to give three fractions (that contains different amounts of cis-impurities). For the next step (was used pure fraction (1.8 g of tert-butyl (2R,5S)-5-methyl-2-(2-methylindazol-6-yl)piperidine-1-carboxylate (8 g, 24.28 mmol, 37.13% yield) as single trans isomer.

LCMS(ESI): [M+H]⁺ m/z: calcd 329.21; found 330.2; Rt=3.691 min.

### Step 6: The Synthesis of tert-butyl (2R,5S)-5-methyl-2-(2-methylindazol-6-yl)piperidine-1-carboxylate

A solution of tert-butyl (2R,5S)-5-methyl-2-(2-methylindazol-6-yl)piperidine-1-carboxylate (1.8 g, 5.46 mmol) in Methanol (20 mL) and Hydrogen chloride solution 4.0M in dioxane (10 g, 274.27 mmol, 12.50 mL) was stirred at 25° C. for 6 hr. The solvent was evaporated to give 2-methyl-6-[(2R,5S)-5-methyl-2-piperidyl]indazole (1.65 g, 5.46 mmol, 100.00% yield, 2HCl).

¹H NMR (400 MHZ, dmso) δ 0.933 (d, 3H), 1.25 (m, 1H), 1.95-1.96 (m, 2H), 2.10 (m, 1H), 2.68 (m, 1H), 3.16 (d, 1H), 4.18 (m, 1H), 7.26 (d, 1H), 7.73 (d, 1H), 7.76 (s, 1H), 8.40 (s, 1H), 9.47 (brs, 2H), 9.77 (brs, 2H).

LCMS(ESI): [M+H]⁺ m/z: calcd 229.1; found 230.2; Rt=0.831 min.

### Step 7: The Synthesis of 2,2,2-trifluoroethyl 2-oxo-2-[(2R,5S)-5-methyl-2-(2-methylindazol-6-yl)-1-piperidyl]acetate

To a solution of 2-methyl-6-[(2R,5S)-5-methyl-2-piperidyl]indazole (1.65 g, 7.20 mmol) and Triethylamine (3.64 g, 35.98 mmol, 5.01 mL) in THF (100 mL), 2,2,2-trifluoroethyl 2-chloro-2-oxo-acetate (4.11 g, 21.59 mmol) was added portionwise at 0° C. The resulting mixture was stirred for 1 hr. The reaction mixture was directly used for the next step.

LCMS(ESI): [M+H]⁺ m/z: calcd 383.1; found 384.2; Rt=1.190 min.

### Step 8: The Synthesis of 2-oxo-2-[(2R,5S)-5-methyl-2-(2-methylindazol-6-yl)-1-piperidyl]acetamide

Through a solution of 2,2,2-trifluoroethyl 2-oxo-2-[(2R,5S)-5-methyl-2-(2-methylindazol-6-yl)-1-piperidyl]acetate (2.7 g, 7.04 mmol) in THF (120 mL), ammonia was bubbled during 10 min at 0° C. The formed precipitate was filtered off, washed with THF (30 ml) and the solvent was evaporated in vacuo to give 2-oxo-2-[(2R,5S)-5-methyl-2-(2-methylindazol-6-yl)-1-piperidyl]acetamide (1.8 g, 5.99 mmol, 85.09% yield).

LCMS(ESI): [M+H]⁺ m/z: calcd 300.1; found 301.2; Rt=0.762 min.

### Step 9: The Synthesis of N-[4-amino-1-(2-trimethylsilylethoxymethyl)pyrazolo[4,3-c]pyridin-7-yl]-2-oxo-2-[(2R,5S)-5-methyl-2-(2-methylindazol-6-yl)-1-piperidyl]acetamide

To a mixture of 2-oxo-2-[(2R,5S)-5-methyl-2-(2-methylindazol-6-yl)-1-piperidyl]acetamide (1 g, 3.33 mmol), 7-bromo-1-(2-trimethylsilylethoxymethyl)pyrazolo[4,3-c]pyridin-4-amine (1.37 g, 4.00 mmol), Copper (42.32 mg, 665.88 μmol), Copper (I) iodide (634.08 mg, 3.33 mmol, 112.83 μL), Cesium carbonate (2.17 g, 6.66 mmol) and (1R,2R)—N1,N2-dimethylcyclohexane-1,2-diamine (710.36 mg, 4.99 mmol), Dioxane (30 mL) was added. The resulting mixture was evacuated, refiled with Argon three time, heated at 100° C. for 36 hr and cooled. The inorganic precipitate was filtered and the solvent was evaporated in vacuo. The residue was purified by gradient chromatography (MTBE-methanol) to give N-[4-amino-1-(2-trimethylsilylethoxymethyl)pyrazolo[4,3-c]pyridin-7-yl]-2-oxo-2-[(2R,5S)-5-methyl-2-(2-methylindazol-6-yl)-1-piperidyl]acetamide (0.4 g, 710.81 μmol, 21.35% yield).

LCMS(ESI): [M+H]⁺ m/z: calcd 562.3; found 563.4; Rt=1.123 min.

### Step 10: The Synthesis of N-(4-amino-1H-pyrazolo[4,3-c]pyridin-7-yl)-2-oxo-2-[(2R,5S)-5-methyl-2-(2-methylindazol-6-yl)-1-piperidyl]acetamide (Compound 180)

A solution of N-[4-amino-1-(2-trimethylsilylethoxymethyl)pyrazolo[4,3-c]pyridin-7-yl]-2-oxo-2-[(2R,5S)-5-methyl-2-(2-methylindazol-6-yl)-1-piperidyl]acetamide (0.4 g, 710.81 μmol) in Trifluoroacetic acid (4 g, 35.08 mmol, 2.70 mL) was stirred at 25° C. for 12 hr. The solvent was evaporated in vacuo and the residue was purified by HPLC (Device (Mobile Phase, Column): SYSTEM 5-5-30% 0-5 min H2O/ACN/0.2% FA, flow: 30 ml/min (loading pump 4 ml/min acetonitrile) target mass 432.49 column: Chromatorex 18 SMB100-5T 100×19 mm 5 um) to give N-(4-amino-1H-pyrazolo[4,3-c]pyridin-7-yl)-2-oxo-2-[(2R,5S)-5-methyl-2-(2-methylindazol-6-yl)-1-piperidyl]acetamide (208 mg, 480.95 μmol, 67.66% yield).

¹H NMR (600 MHz, dmso) δ 0.89-1.20 (m, 3H), 1.25-1.43 (m, 1H), 1.67-1.85 (m, 1H), 1.85-2.03 (m, 1H), 2.04-2.24 (m, 1H), 2.25-2.37 (m, 1H), 2.81-3.23 (m, 1H), 3.38-3.85 (m, 1H), 4.03-4.29 (m, 3H), 5.35-5.77 (m, 1H), 6.90-7.14 (m, 1H), 7.38-7.55 (m, 1H), 7.61-7.73 (m, 1H), 7.82-7.97 (m, 1H), 8.10-8.25 (m, 1H), 8.25-8.32 (m, 1H), 8.32-8.88 (m, 1H), 9.95-11.01 (m, 1H), 12.45 (br s, 1H), 13.62 (br s, 1H).

LCMS(ESI): [M+H]⁺ m/z: calcd 432.2; found 433.2; Rt=1.08 min.

### Example 161. 2-((2R,5S)-5-methyl-2-(2-(1-methyl-1,2,3,6-tetrahydropyridin-4-yl)benzo[d]thiazol-5-yl)piperidin-1-yl)-2-oxo-N-(1H-pyrazolo[4,3-c]pyridin-7-yl)acetamide (Compound 182)

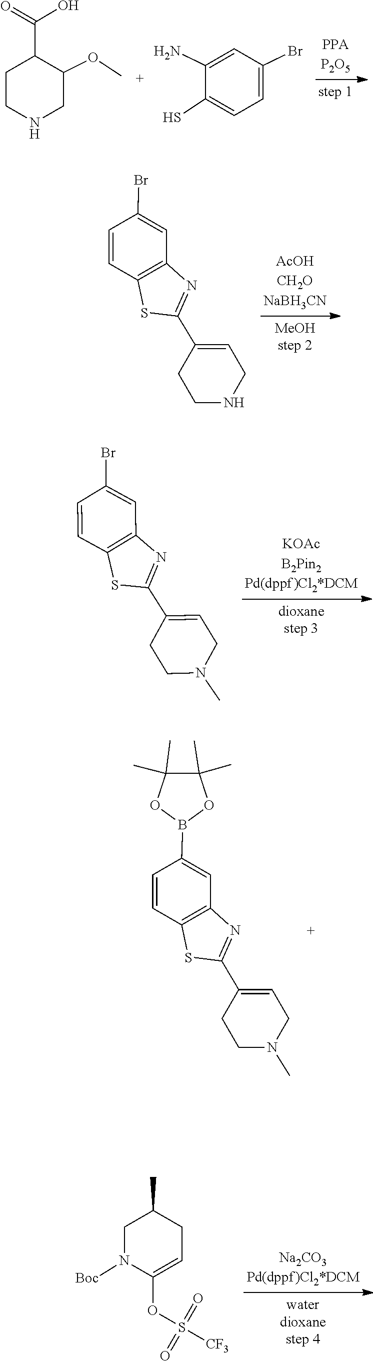

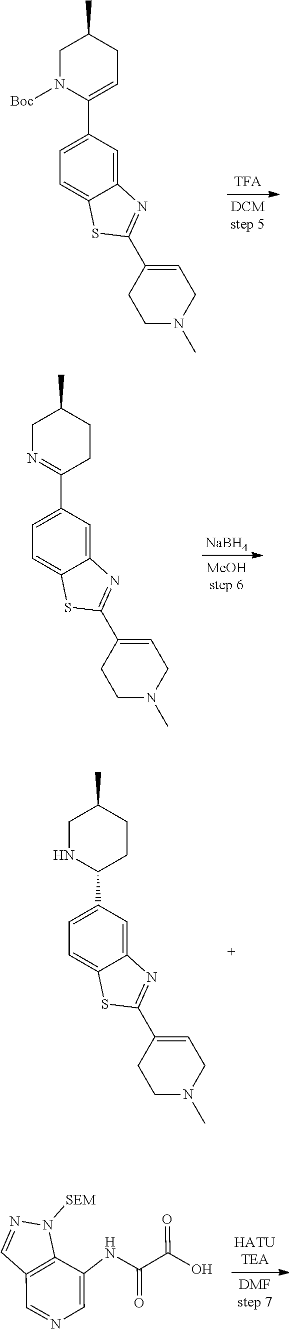 [SMILES: C[C@H]1CC=C(c2ccc3sc(C4=CCN(C)CC4)nc3c2)N(C(=O)OC(C)(C)C)C1 ; C[C@H]1CCC(c2ccc3sc(C4=CCN(C)CC4)nc3c2)=NC1 ; "NaBH4" ; C[C@H]1CC[C@H](c2ccc3sc(C4=CCN(C)CC4)nc3c2)NC1 ; C[Si](C)(C)CCOCn1ncc2cncc(NC(=O)C(=O)O)c21]

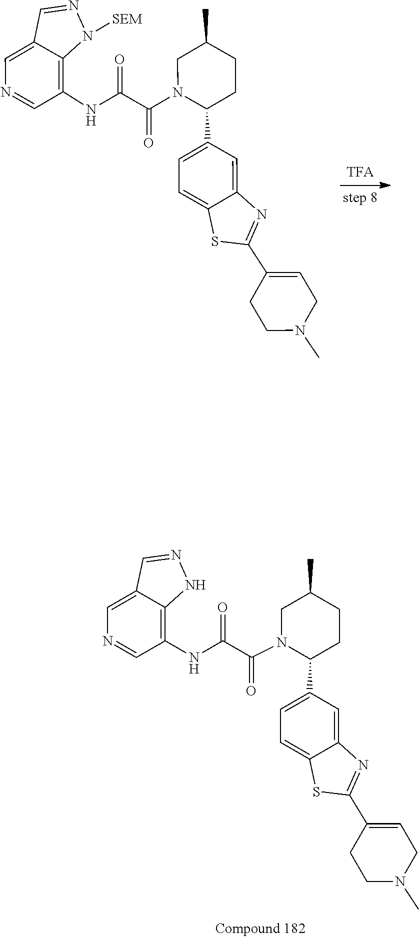 [SMILES: C[C@H]1CC[C@H](c2ccc3sc(C4=CCN(C)CC4)nc3c2)N(C(=O)C(=O)Nc2cncc3cnn(COCC[Si](C)(C)C)c23)C1 ; C[C@H]1CC[C@H](c2ccc3sc(C4=CCN(C)CC4)nc3c2)N(C(=O)C(=O)Nc2cncc3cn[nH]c23)C1]

### Step 1: Synthesis of 5-bromo-2-(1,2,3,6-tetrahydropyridin-4-yl)benzo[d]thiazole

Prepared by general procedure scheme S2 step 1A. Yield: 2.5 g of crude.

LCMS(ESI): [M]⁺ m/z: calcd 295.2; found 296.2; Rt=0.961 min.

### Step 2: Synthesis of 5-bromo-2-(1-methyl-1,2,3,6-tetrahydropyridin-4-yl)benzo[d]thiazole

Formaldehyde, 37% w/w aq. soln., stab. with 7-8% MeOH (1.03 g, 12.70 mmol, 952.01 μL, 37% purity) and acetic acid (1.02 g, 16.94 mmol, 969.60 μL) were added to a stirred solution of 5-bromo-2-(1,2,3,6-tetrahydropyridin-4-yl)-1,3-benzothiazole (2.5 g, 8.47 mmol) in MeOH (100 mL) at 25° C. The resulting mixture was stirred at 25° C. for 1 hr, then sodium cyan borohydride (638.62 mg, 10.16 mmol) was added in one portion at 25° C. (foaming!). The reaction mixture was stirred at 25° C. for 18 hr, and then concentrated in vacuum. The residue was diluted with 10% aqueous sodium hydroxide solution (70 ml) and extracted with DCM (2*60 ml). The combined organic extracts were dried over sodium sulphate and concentrated in vacuum to afford crude 5-bromo-2-(1-methyl-3,6-dihydro-2H-pyridin-4-yl)-1,3-benzothiazole (2.5 g, 8.08 mmol, 95.46% yield) as light-brown gum, which was used directly in the next step.

LCMS(ESI): [M]⁺ m/z: calcd 309.2; found 310.2; Rt=2.242 min.

### Step 3: Synthesis of 2-(1-methyl-1,2,3,6-tetrahydropyridin-4-yl)-5-(4,4,5,5-tetramethyl-1,3,2-dioxaborolan-2-yl)benzo[d]thiazole

Prepared by general procedure scheme S2 step 2. Yield: 2.88 g of crude.

LCMS(ESI): [M]⁺ m/z: calcd 356.2; found 357.2; Rt=1.131 min.

### Step 4: Synthesis of (S)-tert-butyl 3-methyl-6-(2-(1-methyl-1,2,3,6-tetrahydropyridin-4-yl)benzo[d]thiazol-5-yl)-3,4-dihydropyridine-1(2H)-carboxylate

Prepared by general procedure scheme S2 step 3. Yield: 7 g of crude.

LCMS(ESI): [M]⁺ m/z: calcd 425.2; found 426.2; Rt=1.236 min.

### Step 5: Synthesis of (S)-2-(1-methyl-1,2,3,6-tetrahydropyridin-4-yl)-5-(5-methyl-3,4,5,6-tetrahydropyridin-2-yl)benzo[d]thiazole

TFA (29.60 g, 259.60 mmol, 20 mL) was added in one portion to a stirred solution of tert-butyl (3S)-3-methyl-6-[2-(1-methyl-3,6-dihydro-2H-pyridin-4-yl)-1,3-benzothiazol-5-yl]-3,4-dihydro-2H-pyridine-1-carboxylate (7 g, 16.45 mmol) in DCM (20 mL). The resulting solution was stirred at 25° C. for 0.5 hr, and then concentrated in vacuum. The residue was diluted with water (100 ml). The resulting solution of TFA salt of the product was decanted from dark-brown oily residue, which was additionally rinsed with water (2*25 ml). The combined aqueous solution was filtered through a cotton pad to remove traces of oily impurities, then basified to pH 11-12 with 10% aqueous sodium hydroxide solution and extracted with DCM (2*50 ml). The combined organic extracts were dried over sodium sulfate and concentrated in vacuum to afford 2-(1-methyl-3,6-dihydro-2H-pyridin-4-yl)-5-[(3S)-3-methyl-2,3,4,5-tetrahydropyridin-6-yl]-1,3-benzothiazole (2.7 g, 8.30 mmol, 50.44% yield) as brown solid, which was directly used in the next step.

LCMS(ESI): [M]⁺ m/z: calcd 325.2; found 326.2; Rt=0.673 min.

### Step 6: Synthesis of 2-(1-methyl-1,2,3,6-tetrahydropyridin-4-yl)-5-((2R,5S)-5-methylpiperidin-2-yl)benzo[d]thiazole

Prepared by general procedure scheme S2 step 5. Yield: 1.4 g of crude.

LCMS(ESI): [M]⁺ m/z: calcd 327.2; found 328.2; Rt=0.719 min.

### Step 7: Synthesis of 2-((2R,5S)-5-methyl-2-(2-(1-methyl-1,2,3,6-tetrahydropyridin-4-yl)benzo[d]thiazol-5-yl)piperidin-1-yl)-2-oxo-N-(1-((2-(trimethylsilyl)ethoxy)methyl)-1H-pyrazolo[4,3-c]pyridin-7-yl)acetamide

Prepared by general procedure scheme S2 step 6A. Yield: 100 mg (25.35%).

HPLC conditions: Column: XBridge BEH C18 100*19 mm, 5 microM; 0-1-6 min 45-45-75% water-MeCN+0.1% NH₄OH; (loading pump 4 ml/min MeCN).

LCMS(ESI): [M]⁺ m/z: calcd 645.2; found 646.2; Rt=3.057 min.

### Step 8: Synthesis of 2-((2R,5S)-5-methyl-2-(2-(1-methyl-1,2,3,6-tetrahydropyridin-4-yl)benzo[d]thiazol-5-yl)piperidin-1-yl)-2-oxo-N-(1H-pyrazolo[4,3-c]pyridin-7-yl)acetamide (Compound 182)

A solution of 2-oxo-2-[(2R,5S)-5-methyl-2-[2-(1-methyl-3,6-dihydro-2H-pyridin-4-yl)-1,3-benzothiazol-5-yl]-1-piperidyl]-N-[1-(2-trimethylsilylethoxymethyl)pyrazolo[4,3-c]pyridin-7-yl]acetamide (100 mg, 154.83 μmol) in TFA (2.96 g, 25.96 mmol, 2 mL) was stirred at 25° C. for 15 hr. The LCMS of the aliquot showed complete SEM-deprotection. The reaction mixture was concentrated in vacuum, the residue was purified by reverse phase HPLC (column: XBridge C18 100×19 mm, 5 um; mobile phase: 20-70% 0-5 min H₂O/MeOH/0.1% NH₄OH, flow rate: 30 ml/min; (loading pump 4 ml/min MeCN)) to afford Compound 182 2-oxo-N-(1H-pyrazolo[4,3-c]pyridin-7-yl)-2-[(2R,5S)-5-methyl-2-[2-(1-methyl-3,6-dihydro-2H-pyridin-4-yl)-1,3-benzothiazol-5-yl]-1-piperidyl]acetamide (28 mg, 54.30 μmol, 35.07% yield) as light-yellow solid.

Compound 182: ¹H NMR (600 MHZ, DMSO-d₆) δ (ppm) 1.06 (m, 3H), 1.38 (m, 1H), 1.77 (m, 1H), 1.92 (m, 1H), 2.12 (m, 1H), 2.29 (s, 3H), 2.33 (m, 1H), 2.59 (m, 2H), 2.66 (m, 2H), 2.85 (m, 1H), 3.10 (m, 2H), 3.89 (m, 1H), 5.67 (m, 1H), 6.74 (m, 1H), 7.42 (m, 1H), 7.92 (m, 1H), 8.05 (m, 1H), 8.33 (m, 1H), 8.48 (m, 1H), 8.93 (m, 1H), 11.21 (s, 1H), 13.13 (s, 1H).

LCMS(ESI): [M]⁺ m/z: calcd 515.2; found 516.2; Rt=2.054 min.

### Example 162. N-(4-amino-1H-pyrazolo[4,3-c]pyridin-7-yl)-2-((2R,5S)-2-(3-cyanophenyl)-5-methylpiperidin-1-yl)-2-oxoacetamide (Compound 183)

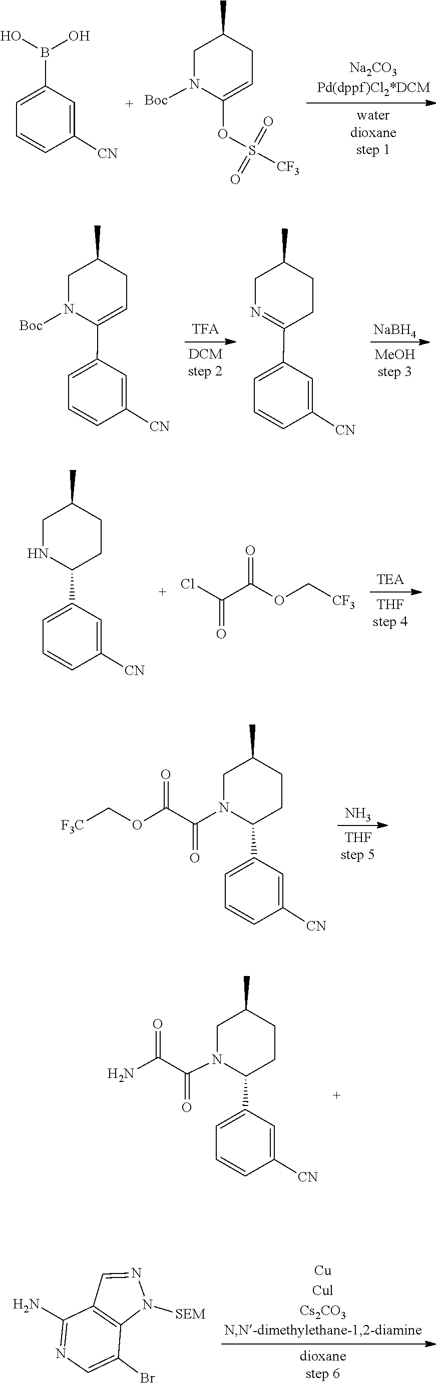

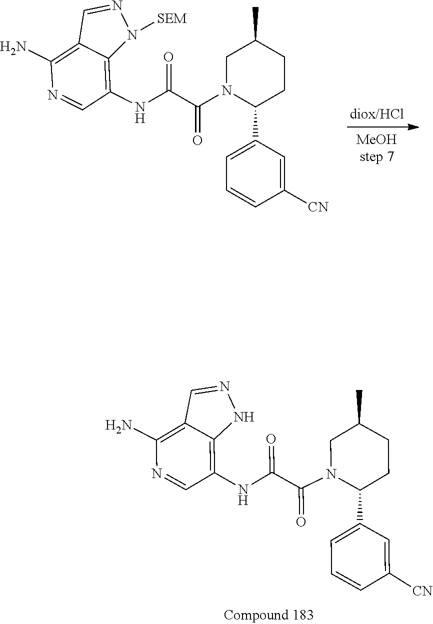 [SMILES: C[C@H]1CC[C@H](c2cccc(C#N)c2)N(C(=O)C(=O)Nc2cnc(N)c3cnn(COCC[Si](C)(C)C)c23)C1 ; C[C@H]1CC[C@H](c2cccc(C#N)c2)N(C(=O)C(=O)Nc2cnc(N)c3cn[nH]c23)C1]

### Step 1: Synthesis of (S)-tert-butyl 6-(3-cyanophenyl)-3-methyl-3,4-dihydropyridine-1(2H)-carboxylate

Prepared by general procedure scheme S8 step 2. Yield: 1.99 g of crude.

LCMS(ESI): [M-Boc]⁺ m/z: calcd 198.2; found 199.2; Rt=4.055 min.

### Step 2: Synthesis of (S)-3-(5-methyl-3,4,5,6-tetrahydropyridin-2-yl)benzonitrile

Prepared by general procedure scheme S8 step 3. Yield: 0.33 g (26.14%).

¹H NMR (500 MHZ, CDCl₃) δ (ppm) 0.97 (d, 3H), 1.39 (m, 1H), 1.70 (m, 1H), 1.93 (m, 1H), 2.54 (m, 1H), 2.70 (m, 1H), 3.27 (m, 1H), 4.02 (m, 1H), 7.43 (m, 1H), 7.81 (m, 1H), 7.99 (m, 1H), 8.05 (m, 1H).

### Step 3: Synthesis of 3-((2R,5S)-5-methylpiperidin-2-yl)benzonitrile

Prepared by general procedure scheme S8 step 4. Yield: 0.3 g of crude.

LCMS(ESI): [M]⁺ m/z: calcd 200.2; found 201.2; Rt=0.578 min.

### Step 4: Synthesis of 2,2,2-trifluoroethyl 2-((2R,5S)-2-(3-cyanophenyl)-5-methylpiperidin-1-yl)-2-oxoacetate

Prepared by general procedure scheme S9 step 1. Yield: 0.53 g of crude.

LCMS(ESI): [M]⁺ m/z: calcd 354.2; found 355.2; Rt=1.169 min.

### Step 5: Synthesis of 2-((2R,5S)-2-(3-cyanophenyl)-5-methylpiperidin-1-yl)-2-oxoacetamide

Prepared by general procedure scheme S9 step 2. Yield: 0.13 g of crude.

LCMS(ESI): [M]⁺ m/z: calcd 271.2; found 272.2; Rt=0.968 min.

### Step 6: Synthesis of N-(4-amino-1-((2-(trimethylsilyl)ethoxy)methyl)-1H-pyrazolo[4,3-c]pyridin-7-yl)-2-((2R,5S)-2-(3-cyanophenyl)-5-methylpiperidin-1-yl)-2-oxoacetamide

Prepared by general procedure scheme S9 step 3A. Yield: 0.25 g of crude.

LCMS(ESI): [M]⁺ m/z: calcd 533.2; found 534.2; Rt=3.395 min.

### Step 7: Synthesis of N-(4-amino-1H-pyrazolo[4,3-c]pyridin-7-yl)-2-((2R,5S)-2-(3-cyanophenyl)-5-methylpiperidin-1-yl)-2-oxoacetamide (Compound 183)

Hydrogen chloride solution 4.0M in dioxane (1.71 g, 46.84 mmol, 2.13 mL) was added to a stirred solution of N-[4-amino-1-(2-trimethylsilylethoxymethyl)pyrazolo[4,3-c]pyridin-7-yl]-2-oxo-2-[(2R,5S)-2-(3-cyanophenyl)-5-methyl-1-piperidyl]acetamide (250.00 mg, 468.43 μmol) in MeOH (1.45 mL) and stirred at 20° C. for 8 hr. Volatiles was evaporated in vacuum, residue dissolved in MeOH (3 mL), quenched with TEA (0.15 g) and submitted to HPLC (column: Chromatorex 18 SMB100-5T 100×19 mm 5 um; 10-10-35% 0-1-5 min H₂O/MeCN/0.1% FA, flow rate: 30 ml/min) to give two main fractions with crude product. 2 fraction was purified by HPLC (column: Chromatorex 18 SMB100-5T 100×19 mm 5 um; 5-5-45% 0-1-5 min H₂O/MeCN/0.1% FA, flow rate: 30 ml/min) to give pure N-(4-amino-1H-pyrazolo[4,3-c]pyridin-7-yl)-2-oxo-2-[(2R,5S)-2-(3-cyanophenyl)-5-methyl-1-piperidyl]acetamide (38 mg, 94.19 μmol, 20.11% yield).

Compound 183: ¹H NMR (600 MHz, DMSO-d₆) δ (ppm) 0.92-1.05 (m, 3H), 1.20-1.33 (m, 1H), 1.59-1.69 (m, 1H), 1.84-1.94 (m, 1H), 2.00-2.12 (m, 1H), 2.14-2.26 (m, 1H), 2.72-3.30 (m, 1H), 3.73-4.14 (m, 1H), 5.04-5.62 (m, 1H), 6.64-6.97 (m, 1H), 7.51-7.65 (m, 2H), 7.67-7.80 (m, 3H), 8.10-8.30 (m, 2H), 9.62-10.62 (m, 1H), 12.52-13.38 (m, 1H).

LCMS(ESI): [M]⁺ m/z: calcd 403.2; found 404.2; Rt=2.485 min.

### Example 163. N-(4-amino-1H-pyrazolo[4,3-c]pyridin-7-yl)-2-(rac-(2,5R)-2-(3-chloro-4-fluorophenyl)-5-methylpiperidin-1-yl)-2-oxoacetamide (Compound 184)

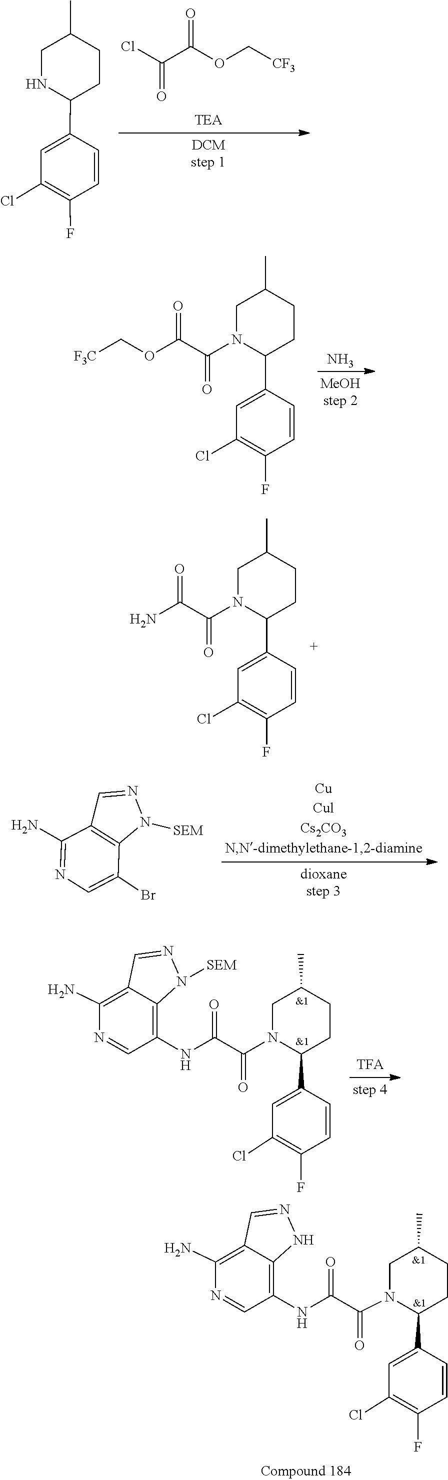

The synthesis of 2-(3-chloro-4-fluoro-phenyl)-5-methyl-piperidine is given by Intermediate 52.

### Step 1: Synthesis of 2,2,2-trifluoroethyl 2-(2-(3-chloro-4-fluorophenyl)-5-methylpiperidin-1-yl)-2-oxoacetate

Prepared by general procedure scheme S9 step 1. Yield: 0.8 g (95.44%).

LCMS(ESI): [M]⁺ m/z: calcd 381.2; found 382.2; Rt=4.330 min.

### Step 2: Synthesis of 2-(2-(3-chloro-4-fluorophenyl)-5-methylpiperidin-1-yl)-2-oxoacetamide

Ammonia (35.69 mg, 2.10 mmol) was bubbled through a solution of 2,2,2-trifluoroethyl 2-[2-(3-chloro-4-fluoro-phenyl)-5-methyl-1-piperidyl]-2-oxo-acetate (0.8 g, 2.10 mmol) in MeOH (25 mL) at rt. After stirring for 18 hr, the reaction mixture was evaporated to dryness to give 2-[2-(3-chloro-4-fluoro-phenyl)-5-methyl-1-piperidyl]-2-oxo-acetamide (0.7 g, crude) as an orange gum.

LCMS(ESI): [M]⁺ m/z: calcd 298.2; found 299.2; Rt=3.164 min.

### Step 3: Synthesis of N-(4-amino-1-((2-(trimethylsilyl)ethoxy)methyl)-1H-pyrazolo[4,3-c]pyridin-7-yl)-2-(rac-(2S,5R)-2-(3-chloro-4-fluorophenyl)-5-methylpiperidin-1-yl)-2-oxoacetamide

Prepared by general procedure scheme S9 step 3A. Yield: 52 mg (13.84%).

HPLC conditions: Column: SunFire C18 100*19 mm, 5 microM; 2-10 min 20-45% water-MeOH+NH₃ 30 ml/min; (loading pump 4 ml/min MeOH+NH₃).

### Step 4: Synthesis of N-(4-amino-1H-pyrazolo[4,3-c]pyridin-7-yl)-2-(rac-(2S,5R)-2-(3-chloro-4-fluorophenyl)-5-methylpiperidin-1-yl)-2-oxoacetamide (Compound 184)

N-[4-Amino-1-(2-trimethylsilylethoxymethyl)pyrazolo[4,3-c]pyridin-7-yl]-2-oxo-2-[rac-(2S,5R)-2-(3-chloro-4-fluoro-phenyl)-5-methyl-1-piperidyl]acetamide (0.052 g, 92.67 μmol) was dissolved in TFA (317.00 mg, 2.78 mmol, 214.19 μL) and stirred at 20° C. for 3 hr. The reaction mixture was submitted to HPLC (2-10 min 35-50% water/MeOH+NH₃ 30/min; loading pump 4 ml/min MeOH column SunFire 19*100 mm). N-(4-Amino-1H-pyrazolo[4,3-c]pyridin-7-yl)-2-oxo-2-[rac-(2S,5R)-2-(3-chloro-4-fluoro-phenyl)-5-methyl-1-piperidyl]acetamide was obtained as a light-yellow solid.

Compound 184: ¹H NMR (600 MHZ, DMSO-d₆) δ (ppm) 0.91-1.07 (m, 3H), 1.26-1.45 (m, 1H), 1.56-1.72 (m, 1H), 1.74-2.10 (m, 2H), 2.13-2.26 (m, 1H), 2.87-3.21 (m, 1H), 3.72-4.11 (m, 1H), 4.97-5.59 (m, 1H), 6.62-7.22 (m, 2H), 7.29-7.47 (m, 2H), 7.47-7.56 (m, 1H), 7.58-7.78 (m, 1H), 8.11-8.30 (m, 1H), 10.57 (s, 1H), 12.79 (s, 1H).

LCMS(ESI): [M]⁺ m/z: calcd 430.2; found 431.2; Rt=2.883 min.

### Example 164. N-(4-amino-1H-pyrazolo[4,3-c]pyridin-7-yl)-2-(5-methyl-2-(pyridin-4-yl)piperidin-1-yl)-2-oxoacetamide (Compound 185 and Compound 186)

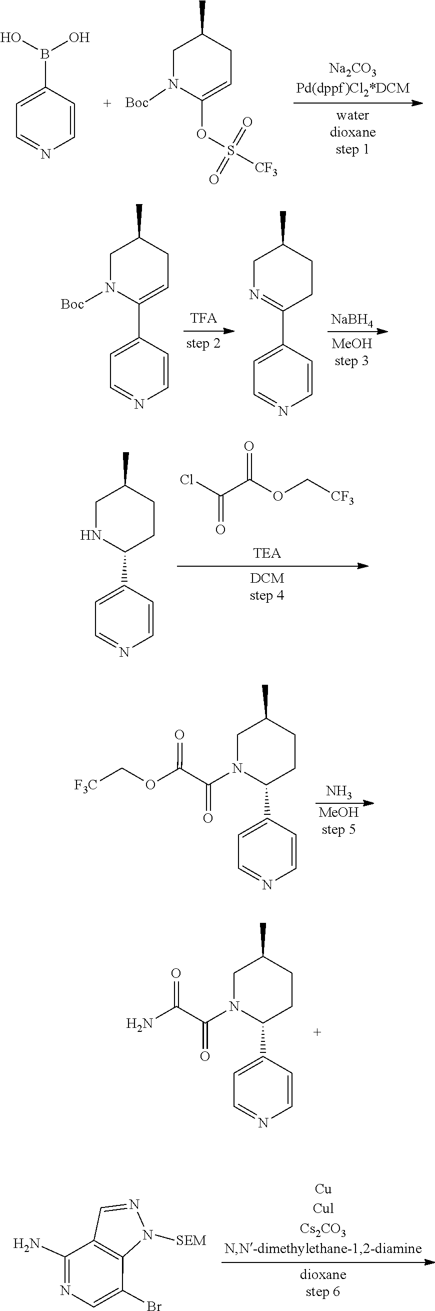

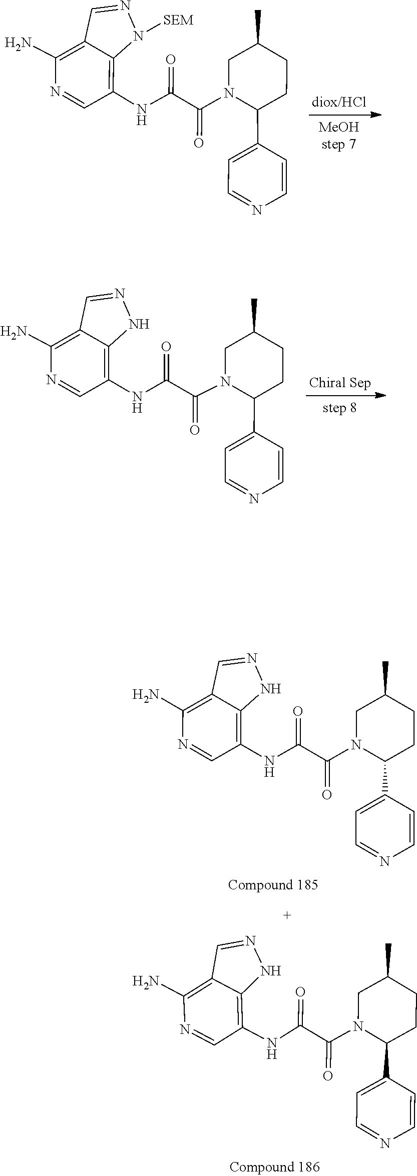 [SMILES: C[C@H]1CCC(c2ccncc2)N(C(=O)C(=O)Nc2cnc(N)c3cnn(COCC[Si](C)(C)C)c23)C1 ; C[C@H]1CCC(c2ccncc2)N(C(=O)C(=O)Nc2cnc(N)c3cn[nH]c23)C1 ; C[C@H]1CC[C@H](c2ccncc2)N(C(=O)C(=O)Nc2cnc(N)c3cn[nH]c23)C1 ; C[C@H]1CC[C@@H](c2ccncc2)N(C(=O)C(=O)Nc2cnc(N)c3cn[nH]c23)C1]

### Step 1: Synthesis of (S)-tert-butyl 5-methyl-5,6-dihydro-[2,4′-bipyridine]-1 (4H)-carboxylate

Prepared by general procedure scheme S8 step 2. Yield: 0.6 g of crude.

LCMS(ESI): [M-Boc]⁺ m/z: calcd 174.2; found 175.2; Rt=1.064 min.

### Step 2: Synthesis of (S)-5-methyl-3,4,5,6-tetrahydro-2,4′-bipyridine

Prepared by general procedure scheme S8 step 3. Yield: 0.4 g of crude.

LCMS(ESI): [M]⁺ m/z: calcd 174.2; found 175.2; Rt=0.514 min.

### Step 3: Synthesis of 4-((2R,5S)-5-methylpiperidin-2-yl)pyridine

Prepared by general procedure scheme S8 step 4. Yield: 0.4 g of crude.

LCMS(ESI): [M]⁺ m/z: calcd 176.2; found 177.2; Rt=0.356 min.

### Step 4: Synthesis of 2,2,2-trifluoroethyl 2-((2R,5S)-5-methyl-2-(pyridin-4-yl)piperidin-1-yl)-2-oxoacetate

Prepared by general procedure scheme S9 step 1. Yield: 1.6 g of crude.

¹H NMR (500 MHz, CDCl₃) δ (ppm) 1.03 (d, 3H), 1.45 (m, 1H), 1.73 (m, 1H), 1.90 (m, 2H), 2.17 (m, 1H), 3.29 (m, 1H), 4.61 (m, 2H), 4.78 (m, 1H), 5.72 (m, 1H), 7.14 (m, 2H), 8.59 (m, 2H).

### Step 5: Synthesis of 2-((2R,5S)-5-methyl-2-(pyridin-4-yl)piperidin-1-yl)-2-oxoacetamide

Prepared by general procedure scheme S9 step 2. Yield: 687.4 mg (57.38%).

LCMS(ESI): [M]⁺ m/z: calcd 247.2; found 248.2; Rt=1.383 min.

### Step 6: Synthesis of N-(4-amino-1-((2-(trimethylsilyl)ethoxy)methyl)-1H-pyrazolo[4,3-c]pyridin-7-yl)-2-((5S)-5-methyl-2-(pyridin-4-yl)piperidin-1-yl)-2-oxoacetamide

Prepared by general procedure scheme S9 step 3A. Yield: 360 mg (49.91%).

HPLC conditions: Column: XBridge C18 100*19 mm, 5 microM; 0-5 min 30-45% water-MeCN+0.1% NH₄OH 30 ml/min; (loading pump 4 ml/min MeOH).

LCMS(ESI): [M]⁺ m/z: calcd 509.2; found 510.2; Rt=3.398 min.

### Step 7: Synthesis of N-(4-amino-1H-pyrazolo[4,3-c]pyridin-7-yl)-2-(5-methyl-2-(pyridin-4-yl)piperidin-1-yl)-2-oxoacetamide

Hydrogen chloride solution 4.0M in dioxane (4.20 g, 16.01 mmol, 5.25 mL, 13.9% purity) was added to a stirred solution of N-[4-amino-1-(2-trimethylsilylethoxymethyl)pyrazolo[4,3-c]pyridin-7-yl]-2-oxo-2-[(5S)-5-methyl-2-(4-pyridyl)-1-piperidyl]acetamide (360 mg, 706.33 μmol) in MeOH (4 mL) at 25° C. The resulting solution was stirred at 25° C. for 20 hr, then concentrated to dryness in vacuum and the residue was submitted to reverse phase HPLC (column: Chromatorex 18 SMB 100-ST 100*19 mm 5 um; mobile phase: 5-5-25% 0-1-5 min H₂O/MeOH/0.1% FA, flow: 30 ml/min (loading pump 4 ml/min H₂O)) to afford 2 fractions of product N-(4-amino-1H-pyrazolo[4,3-c]pyridin-7-yl)-2-oxo-2-[(5S)-5-methyl-2-(4-pyridyl)-1-piperidyl]acetamide (154 mg, 361.98 μmol, 51.25% yield, HCOOH) as mixture of cis/trans-isomers (154 mg, yellow solid), which were combined and submitted to preparative chiral HPLC (next step).

LCMS(ESI): [M]⁺ m/z: calcd 379.2; found 380.2; Rt=0.693 min.

### Step 8: Chiral Separation (Compound 185 and Compound 186)

Racemic N-(4-amino-1H-pyrazolo[4,3-c]pyridin-7-yl)-2-oxo-2-[(5S)-5-methyl-2-(4-pyridyl)-1-piperidyl]acetamide (154 mg, 405.89 μmol) was chiral separated (Column: CHIRALPAK IC (250×30 mm, 10 mkm); Mobile Phase: Hexane:IPA:MeOH:DEA, 50:25:25:0.1; Flow Rate: 40 ml/min) to obtain N-(4-amino-1H-pyrazolo[4,3-c]pyridin-7-yl)-2-oxo-2-[(2R,5S)-5-methyl-2-(4-pyridyl)-1-piperidyl]acetamide (120.6 mg, 317.86 μmol, 78.31% yield) (RT=39.79 min) and N-(4-amino-1H-pyrazolo[4,3-c]pyridin-7-yl)-2-oxo-2-[(2S,5S)-5-methyl-2-(4-pyridyl)-1-piperidyl]acetamide (37.8 mg, 99.63 μmol, 24.55% yield) (RT=27.31 min).

Rel Time for Compound 185 in analytical conditions (column: IC, Hexane (0.1% EDA)-IPA-MeOH, 50-25-25, 0.6 ml/min as mobile phase) 51.85 min and for Compound 186 32.85 min.

Compound 185: Retention time: 51.85 min.

¹H NMR (600 MHz, DMSO-d₆) δ (ppm) 0.94-1.06 (m, 3H), 1.31-1.63 (m, 2H), 1.68-2.26 (m, 3H), 2.72-3.27 (m, 1H), 3.45-4.16 (m, 1H), 5.00-5.64 (m, 1H), 6.21-6.83 (m, 2H), 6.99-7.38 (m, 2H), 7.51-7.72 (m, 1H), 8.12-8.21 (m, 1H), 8.24-8.60 (m, 2H), 9.62-10.57 (m, 1H), 12.65-13.41 (m, 1H).

LCMS(ESI): [M]⁺ m/z: calcd 379.2; found 380.2; Rt=0.592 min.

Compound 186: Retention time: 32.85 min.

LCMS(ESI): [M]⁺ m/z: calcd 379.2; found 380.2; Rt=0.616 min.

### Example 165. 2-((2R,5S)-2-(1H-indazol-5-yl)-5-methylpiperidin-1-yl)-N-(4-amino-2H-pyrazolo[4,3-c]pyridin-7-yl)-2-oxoacetamide (Compound 190)

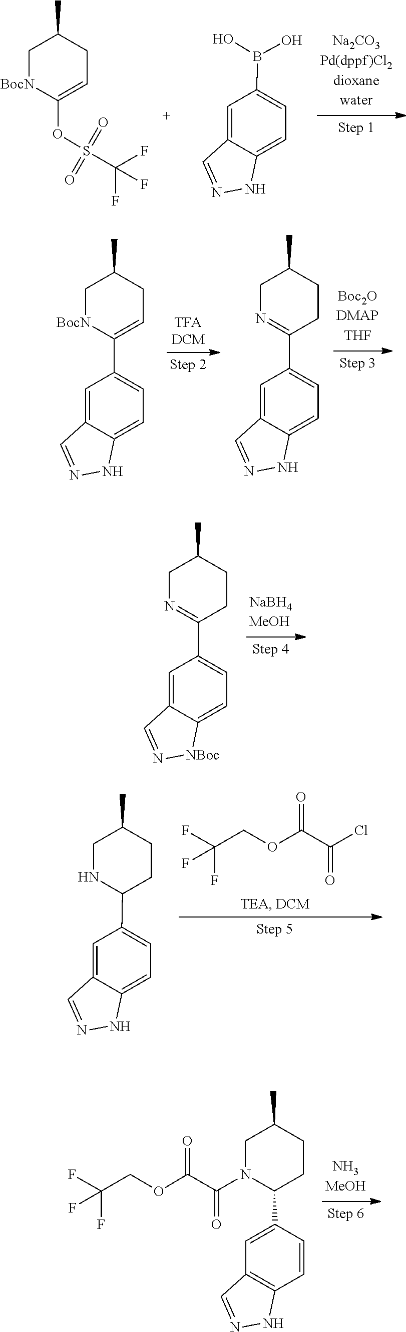

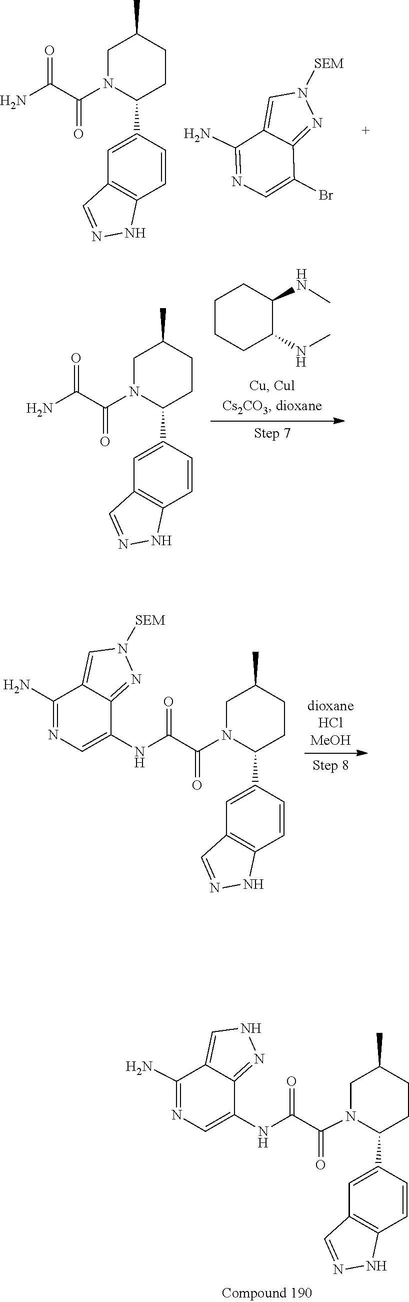

### Step 1: (S)-tert-butyl 6-(1H-indazol-5-yl)-3-methyl-3,4-dihydropyridine-1(2H)-carboxylate

5-(4,4,5,5-tetramethyl-1,3,2-dioxaborolan-2-yl)-1H-indazole (2 g, 7.13 mmol, HCl) and(S)-tert-butyl 3-methyl-6-(((trifluoromethyl) sulfonyl)oxy)-3,4-dihydropyridine-1(2H)-carboxylate (4.06 g, 11.76 mmol) were mixed together in the dioxane (39.91 mL) and sodium carbonate (3.02 g, 28.51 mmol, 1.19 mL) (solution in the H₂O (15.00 mL)) was added thereto. The resulting mixture was evacuated and backfilled with argon, this operation was repeated three times.

Cyclopentyl(diphenyl)phosphane; dichloromethane; dichloropalladium; iron (291.08 mg, 356.43 μmol) was added to the reaction mixture under argon stream and this reaction mixture was stirred at 100° C. for 60 hr. After completion the reaction mixture was cooled to the room temperature and solvents were evaporated under reduced pressure. Water (80 mL) was added to the residue, stirred for 30 min and the precipitate was filtered off, washed with water and dried to afford(S)-tert-butyl 6-(1H-indazol-5-yl)-3-methyl-3,4-dihydropyridine-1(2H)-carboxylate (2.5 g, crude) as a black oil which was used in the next step without further purification.

LCMS(ESI): [M+H]⁺ m/z: calcd 313.19; found 314.2; Rt=1.363 min.

### Step 2: (S)-5-(5-methyl-3,4,5,6-tetrahydropyridin-2-yl)-1H-indazole

(S)-tert-butyl 6-(1H-indazol-5-yl)-3-methyl-3,4-dihydropyridine-1(2H)-carboxylate (2.5 g, 7.98 mmol) was dissolved in the DCM (70 mL) and TFA (9.10 g, 79.77 mmol, 6.15 mL) was added thereto. This reaction mixture was stirred at 25° C. for 18 hr. After completion the solvent was evaporated under reduced pressure and the residue was diluted with warm water (40 mL) and this solution was decanted from the black resin. K₂CO₃ was added to the aqueous solution to alkaline reaction and the formed precipitate was filtered off and washed with water (2×5 mL) to get(S)-5-(5-methyl-3,4,5,6-tetrahydropyridin-2-yl)-1H-indazole (0.6 g, 2.81 mmol, 35.27% yield) as a white solid which was used in the next step without further purification.

LCMS(ESI): [M+H]⁺ m/z: calcd 213.14; found 214.2; Rt=0.562 min.

### Step 3: (S)-tert-butyl 5-(5-methyl-3,4,5,6-tetrahydropyridin-2-yl)-1H-indazole-1-carboxylate

To a mixture of (S)-5-(5-methyl-3,4,5,6-tetrahydropyridin-2-yl)-1H-indazole (0.6 g, 2.81 mmol) and DMAP (34.37 mg, 281.32 μmol) in THF (19.35 mL), tert-butoxycarbonyl tert-butyl carbonate (613.98 mg, 2.81 mmol, 645.61 μL) was added dropwise and the resulting mixture was stirred at r.t overnight. Upon completion of the reaction, solvent was evaporated to dryness, residue was dissolved in CHCl₃, washed with NaHSO₄ water solution (0.5 g in 50 mL of water), organic layer was dried and evaporated to obtain(S)-tert-butyl 5-(5-methyl-3,4,5,6-tetrahydropyridin-2-yl)-1H-indazole-1-carboxylate (0.6 g, crude).

LCMS(ESI): [M+H]⁺ m/z: calcd 313.19; found 314.2; Rt=0.776 min.

### Step 4:5-((5S)-5-methylpiperidin-2-yl)-1H-indazole

To a mixture of (S)-tert-butyl 5-(5-methyl-3,4,5,6-tetrahydropyridin-2-yl)-1H-indazole-1-carboxylate (0.3 g, 957.26 μmol) in MeOH (14.95 mL) was cooled to 0° C., sodium Borohydride (54.32 mg, 1.44 mmol, 50.58 μL) was added in one portion. The resulting mixture was allowed to stir overnight at 25° C. Solvent was evaporated to dryness, water (20 mL) was added and the resulting mixture was basified with K₂CO₃ to alkaline pH and extracted with DCM (3×15 mL), combined organic layers were dried and concentrated in vacuo to obtain 5-((5S)-5-methylpiperidin-2-yl)-1H-indazole (0.2 g, crude) as a yellow oil.

LCMS(ESI): [M+H]⁺ m/z: calcd 315.2; found 316.2; Rt=0.875 min.

### Step 5:2,2,2-trifluoroethyl 2-((2R,5S)-2-(1H-indazol-5-yl)-5-methylpiperidin-1-yl)-2-oxoacetate

5-((5S)-5-methylpiperidin-2-yl)-1H-indazole (0.3 g, 1.39 mmol) and TEA (282.01 mg, 2.79 mmol, 388.44 μL) were mixed together in DCM (19.88 mL) and the resulting solution was cooled to 5° C. in an ice bath. 2,2,2-trifluoroethyl 2-chloro-2-oxo-acetate (292.00 mg, 1.53 mmol) was added dropwise to the previous solution and the resulting mixture was allowed to warm to rt and stirred for 20 hr. The mixture was washed with brine (2×20 mL), organic layer was dried over anhydrous sodium sulfate and filtered. The filtrate was concentrated in vacuo. The resulting crude product was dissolved in DCM (19.88 mL), TEA (282.01 mg, 2.79 mmol, 388.44 μL) and 2,2,2-trifluoroethyl 2-chloro-2-oxo-acetate (292.00 mg, 1.53 mmol) was added dropwise, the resulting mixture was stirred for 3 hours, then mixture was washed with brine (2×20 mL), organic layer was dried over anhydrous sodium sulfate and filtered. The filtrate was concentrated in vacuo to afford 2,2,2-trifluoroethyl 2-((2R,5S)-2-(1H-indazol-5-yl)-5-methylpiperidin-1-yl)-2-oxoacetate (0.3 g, crude) which was used in the next step without further purification.

LCMS(ESI): [M+H]⁺ m/z: calcd 369.14; found 370.2; Rt=1.167 min.

### Step 6:2-((2R,5S)-2-(1H-indazol-5-yl)-5-methylpiperidin-1-yl)-2-oxoacetamide

2,2,2-trifluoroethyl 2-((2R,5S)-2-(1H-indazol-5-yl)-5-methylpiperidin-1-yl)-2-oxoacetate (236.02 mg, 639.04 μmol) was dissolved in NH₃/MeOH (20 mL). The resulting clear solution was left for 15 hr at 25° C. Upon completion of the reaction the resulting mixture was concentrated to dryness to obtain 2-((2R,5S)-2-(1H-indazol-5-yl)-5-methylpiperidin-1-yl)-2-oxoacetamide (0.21 g, crude) as a brown solid.

LCMS(ESI): [M+H]⁺ m/z: calcd 286.15; found 287.2; Rt=0.976 min.

### Step 7:2-((2R,5S)-2-(1H-indazol-5-yl)-5-methylpiperidin-1-yl)-N-(4-amino-2-((2-(trimethylsilyl)ethoxy)methyl)-2H-pyrazolo[4,3-c]pyridin-7-yl)-2-oxoacetamide

A mixture of 2-((2R,5S)-2-(1H-indazol-5-yl)-5-methylpiperidin-1-yl)-2-oxoacetamide (210 mg, 733.42 μmol), 7-bromo-2-(2-trimethylsilylethoxymethyl)pyrazolo[4,3-c]pyridin-4-amine (276.96 mg, 806.77 μmol), cu (9.32 mg, 146.68 μmol), cesium carbonate (358.45 mg, 1.10 mmol), CuI (27.94 mg, 146.68 μmol, 4.97 μL) and (1S,2S)—N,N′-Bis-methyl-1,2-cyclohexane-diamine (20.86 mg, 146.68 μmol, 23.13 μL) in dioxane (5.03 mL) was stirred in a sealed vial under argon at 105° C. for 24 hr. After that time, solvent was evaporated to dryness to afford crude product, then crude product was subjected by HPLC (column: Chromatorex 18 SMB100-5T 100×19 mm, 5 um; mobile phase: 10-10-70% 0-1-5 min H₂O/ACN/0.1% FA, flow rate: 30 ml/min (loading pump 4 ml/min ACN) to obtain 2-((2R,5S)-2-(1H-indazol-5-yl)-5-methylpiperidin-1-yl)-N-(4-amino-2-((2-(trimethylsilyl)ethoxy)methyl)-2H-pyrazolo[4,3-c]pyridin-7-yl)-2-oxoacetamide (108.1 mg, crude) as a brown oil.

LCMS(ESI): [M+H]⁺ m/z: calcd 548.32; found 549.2; Rt=0.974 min.

### Step 8: The synthesis of 2-((2R,5S)-2-(1H-indazol-5-yl)-5-methylpiperidin-1-yl)-N-(4-amino-2H-pyrazolo[4,3-c]pyridin-7-yl)-2-oxoacetamide

2-((2R,5S)-2-(1H-indazol-5-yl)-5-methylpiperidin-1-yl)-N-(4-amino-2-((2-(trimethylsilyl)ethoxy)methyl)-2H-pyrazolo[4,3-c]pyridin-7-yl)-2-oxoacetamide (0.108 g, 196.83 μmol) was dissolved in a mixture of MeOH (2 mL) and dioxane/HCl (2 mL). The resulting mixture was stirred at 25° C. for 3 hr. After that time, solvent was evaporated to dryness to afford crude product, then crude product was subjected by HPLC (column: Chromatorex 18 SMB100-5T 100×19 mm, 5 um; mobile phase: 20-45% 0-5 min H₂O/ACN/0.1% FA, flow rate: 30 ml/min (loading pump 4 ml/min ACN)), then repurified by HPLC (column: Kinetex PFP 100×21.2 mm 5 um; mobile phase: 20-45% 0-5 min H₂O/ACN/0.1% FA, flow rate: 30 ml/min (loading pump 4 ml/min ACN) to obtain 2-((2R,5S)-2-(1H-indazol-5-yl)-5-methylpiperidin-1-yl)-N-(4-amino-2H-pyrazolo[4,3-c]pyridin-7-yl)-2-oxoacetamide (5.7 mg, 12.27 μmol, 6.23% yield, HCOO—) as a brown solid.

¹H NMR (600 MHz, dmso) δ 0.71-1.10 (m, 3H), 1.26-1.54 (m, 1H), 1.54-1.96 (m, 2H), 1.98-2.24 (m, 1H), 2.24-2.36 (m, 1H), 2.74-3.05 (m, 1H), 3.75-4.14 (m, 1H), 5.05-5.91 (m, 1H), 6.57-7.07 (m, 2H), 7.20-7.45 (m, 1H), 7.52-7.74 (m, 2H), 7.81-8.09 (m, 1H), 8.09-8.38 (m, 2H), 9.54-10.58 (m, 1H), 12.58-13.42 (m, 2H).

LCMS(ESI): [M+H]⁺ m/z: calcd 418.21; found 419.2; Rt=1.823 min.

### Example 166. N-(4-Amino-1H-pyrazolo[4,3-c]pyridin-7-yl)-2-oxo-2-[rac-(2R,5S)-2-(5-chloro-3-pyridyl)-5-methyl-1-piperidyl]acetamide (Compound 203)

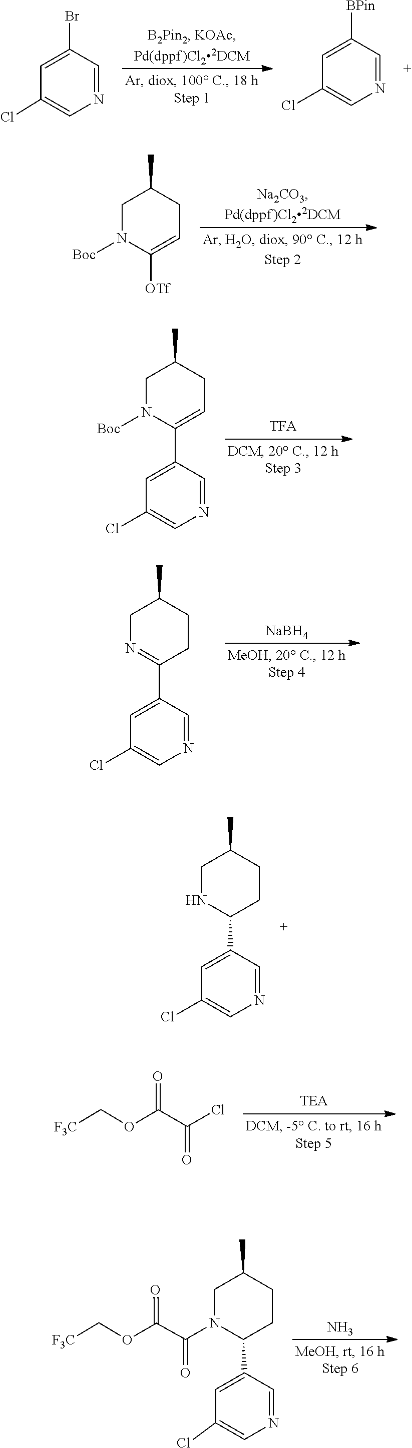

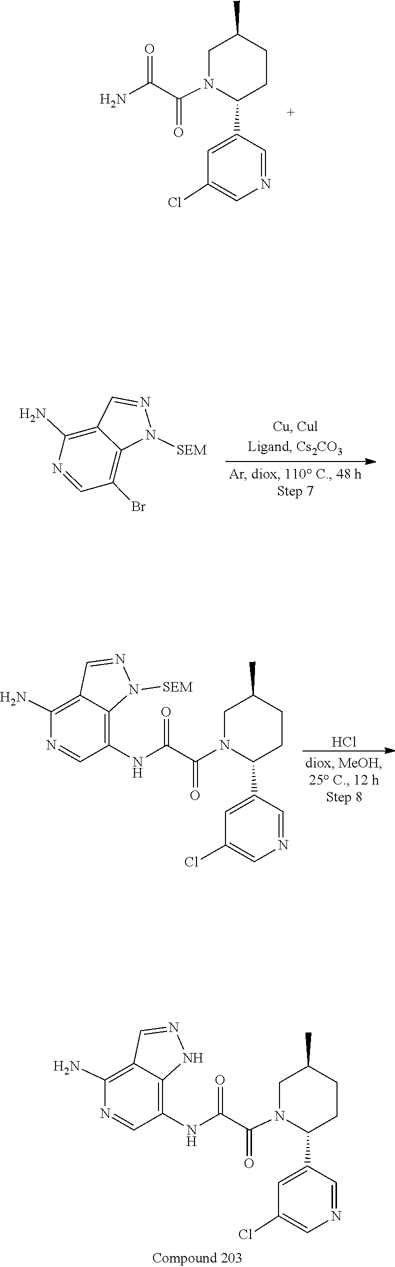 [SMILES: C[C@H]1CC[C@H](c2cncc(Cl)c2)N(C(=O)C(N)=O)C1 ; C[Si](C)(C)CCOCn1ncc2c(N)ncc(Br)c21 ; C[C@H]1CC[C@H](c2cncc(Cl)c2)N(C(=O)C(=O)Nc2cnc(N)c3cnn(COCC[Si](C)(C)C)c23)C1 ; Cl ; C[C@H]1CC[C@H](c2cncc(Cl)c2)N(C(=O)C(=O)Nc2cnc(N)c3cn[nH]c23)C1]

### Step 1: The Synthesis of 3-Chloro-5-(4,4,5,5-tetramethyl-1,3,2-dioxaborolan-2-yl)pyridine

3-Bromo-5-chloro-pyridine (5 g, 25.98 mmol), Pd(dppf)Cl2·DCM (1.06 g, 1.30 mmol), Potassium Acetate (7.65 g, 77.95 mmol, 4.87 mL) and Bis(pinacolato)diboron (7.26 g, 28.58 mmol) were mixed together in dioxane (40 mL) and the resulting mixture was evacuated and backfilled three times with argon, then the resulting mixture was heated at 100° C. (oil bath) overnight. The reaction mixture was cooled and filtered, the filtrate was concentrated in vacuo. The crude product was purified by FCC (MTBE in hexanes from 12% to 95%). 3-Chloro-5-(4,4,5,5-tetramethyl-1,3,2-dioxaborolan-2-yl)pyridine (3.2 g, 13.36 mmol, 51.42% yield) was obtained as a light-yellow solid.

### Step 2: The Synthesis of tert-Butyl rac-(3S)-6-(5-chloro-3-pyridyl)-3-methyl-3,4-dihydro-2H-pyridine-1-carboxylate

3-Chloro-5-(4,4,5,5-tetramethyl-1,3,2-dioxaborolan-2-yl)pyridine (3.2 g, 13.36 mmol), tert-butyl rac-(3S)-3-methyl-6-(trifluoromethylsulfonyloxy)-3,4-dihydro-2H-pyridine-1-carboxylate (5.08 g, 14.70 mmol), Sodium carbonate (2.83 g, 26.72 mmol, 1.12 mL) and Pd(dppf)Cl2·DCM (545.35 mg, 667.80 μmol) were mixed in dioxane (30 mL)+water (7 mL) under argon, and then stirred overnight at 90° C. for 12 hr in vial. The solid was filtered off, the filtrate was diluted with EA, the organic layer was separated and the aqueous layer was extracted with additional EA, the combined organic layer was washed with brine, dried over Na₂SO₄, filtered and concentrated in vacuo. The obtained product was used in the next step without further purification. The reaction was successful. tert-Butyl rac-(3S)-6-(5-chloro-3-pyridyl)-3-methyl-3,4-dihydro-2H-pyridine-1-carboxylate (5.91 g, crude) was obtained as a red gum.

LCMS(ESI): [M+H]⁺ m/z: calcd 309.2; found 309.2; Rt=1.521 min.

### Step 3: The Synthesis of 3-Chloro-5-[rac-(3S)-3-methyl-2,3,4,5-tetrahydropyridin-6-yl]pyridine

tert-Butyl rac-(3S)-6-(5-chloro-3-pyridyl)-3-methyl-3,4-dihydro-2H-pyridine-1-carboxylate (5.91 g, 19.14 mmol) was dissolved in a mixture of DCM (25 mL) and TFA (25 mL). The resulting clear solution was stirred for 12 hr at 20° C. The reaction mixture was concentrated on vacuo. The obtained residue was dissolved in water and extracted with MTBE (3 times), after that the aqueous layer was basified with NaOH and extracted with DCM (3 times), combined DCM layers was dried over Na₂SO₄, filtered and evaporated. The reaction is successful. 3-Chloro-5-[rac-(3S)-3-methyl-2,3,4,5-tetrahydropyridin-6-yl]pyridine (1.9 g, 9.10 mmol, 47.57% yield) was obtained as a red liquid.

LCMS(ESI): [M+H]⁺ m/z: calcd 209.0; found 209.0; Rt=0.538 min.

### Step 4: The Synthesis of 3-Chloro-5-[rac-(2R,5S)-5-methyl-2-piperidyl]pyridine

To a stirring suspension of 3-chloro-5-[rac-(3S)-3-methyl-2,3,4,5-tetrahydropyridin-6-yl]pyridine (1.9 g, 9.10 mmol) in MeOH (40 mL) was added Sodium Borohydride (688.90 mg, 18.21 mmol, 641.43 μL) in portions. Resulting mixture was stirred at 20° C. for 12 hr. The solvent was removed under reduced pressure and residue was partitioned between water and DCM. The organic layer was separated, dried over Na₂SO₄ and concentrated in vacuo. The obtained product was used in the next step without further purification. The reaction is successful. 3-Chloro-5-[rac-(2R,5S)-5-methyl-2-piperidyl]pyridine (1.1 g, 5.22 mmol, 57.34% yield) was obtained as a red oil.

LCMS(ESI): [M+H]⁺ m/z: calcd 211.2; found 211.2; Rt=0.700 min.

### Step 5: The Synthesis of 2,2,2-Trifluoroethyl 2-oxo-2-[rac-(2R,5S)-2-(5-chloro-3-pyridyl)-5-methyl-1-piperidyl]acetate

3-Chloro-5-[rac-(2R,5S)-5-methyl-2-piperidyl]pyridine (1.1 g, 5.22 mmol) was dissolved in DCM (15 mL) and Triethylamine (607.52 mg, 6.00 mmol, 836.80 μL) was added thereto. The resulting mixture was cooled to −5° C. in an ice/methanol bath and a solution of 2,2,2-trifluoroethyl 2-chloro-2-oxo-acetate (1.09 g, 5.74 mmol) in DCM (5 mL) was added dropwise. The resulting mixture was allowed to warm to room temperature and stirred overnight. Water (15 mL) was added to the reaction mixture and an organic layer was separated. The aqueous layer was extracted with DCM (25 mL) and combined organic layers were washed with aq. NaHCO₃ (25 mL), dried over Na₂SO₄, filtered and concentrated in vacuo to obtain 2,2,2-trifluoroethyl 2-oxo-2-[rac-(2R,5S)-2-(5-chloro-3-pyridyl)-5-methyl-1-piperidyl]acetate (1.7 g, 4.66 mmol, 89.28% yield).

LCMS(ESI): [M+H]⁺ m/z: calcd 365.2; found 365.2; Rt=1.343 min.

### Step 6: The Synthesis of 2-Oxo-2-[rac-(2R,5S)-2-(5-chloro-3-pyridyl)-5-methyl-1-piperidyl]acetamide

2,2,2-Trifluoroethyl 2-oxo-2-[rac-(2R,5S)-2-(5-chloro-3-pyridyl)-5-methyl-1-piperidyl]acetate (1.7 g, 4.66 mmol) was dissolved in NH₃/MeOH (50 mL) and the resulting mixture was stirred overnight. The reaction mixture was concentrated in vacuo to obtain 2-oxo-2-[rac-(2R,5S)-2-(5-chloro-3-pyridyl)-5-methyl-1-piperidyl]acetamide (1.37 g, crude).

LCMS(ESI): [M+H]⁺ m/z: calcd 282.2; found 282.2; Rt=0.976 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-2-(5-chloro-3-pyridyl)-5-methyl-1-piperidyl]acetamide

Copper (2.26 mg, 35.49 μmol), Copper (I) iodide (67.60 mg, 354.94 μmol, 12.03 μL), caesium carbonate (462.59 mg, 1.42 mmol) was added to a stirred solution of 2-oxo-2-[rac-(2R,5S)-2-(5-chloro-3-pyridyl)-5-methyl-1-piperidyl]acetamide (0.2 g, 709.88 μmol), 7-bromo-1-(2-trimethylsilylethoxymethyl)pyrazolo[4,3-c]pyridin-4-amine (255.88 mg, 745.37 μmol), rac-(1R,2R)—N1,N2-dimethylcyclohexane-1,2-diamine (196.90 mg, 1.38 mmol) in 1,4-dioxane (5.00 mL) under Ar atmosphere and stirred at 110° C. for 48 hr in closed vial. Reaction mixture was filtered, solid washed with dioxane (2×3 mL), filtrate concentrated and purified by HPLC (column: Chromatorex 18 SMB100-5T 100×19 mm 5 um; 20-45% 0-5 min H₂O/ACN/0.1% FA, flow rate: 30 mL/min) to give pure N-[4-amino-1-(2-trimethylsilylethoxymethyl)pyrazolo[4,3-c]pyridin-7-yl]-2-oxo-2-[rac-(2R,5S)-2-(5-chloro-3-pyridyl)-5-methyl-1-piperidyl]acetamide (82 mg, 150.70 μmol, 21.23% yield).

LCMS(ESI): [M+H]⁺ m/z: calcd 544.2; found 544.2; Rt=0.992 min.

### Step 8: The Synthesis of N-(4-Amino-1H-pyrazolo[4,3-c]pyridin-7-yl)-2-oxo-2-[rac-(2R,5S)-2-(5-chloro-3-pyridyl)-5-methyl-1-piperidyl]acetamide (Compound 203)

Hydrogen chloride solution 4.0M in dioxane (824.19 mg, 22.61 mmol, 1.03 mL) was added to a solution of N-[4-amino-1-(2-trimethylsilylethoxymethyl)pyrazolo[4,3-c]pyridin-7-yl]-2-oxo-2-[rac-(2R,5S)-2-(5-chloro-3-pyridyl)-5-methyl-1-piperidyl]acetamide (82 mg, 150.70 μmol) in Methanol (999.76 μL) and stirred at 25° C. for 12 hr. Volatiles was evaporated in vacuo, residue dissolved in Methanol (2 mL), neutralized with TEA (70 mg) and submitted to HPLC (column: Chromatorex 18 SMB 100-ST 100*19 mm 5 um; 10-10-35% 0-1-6 min H₂O/ACN/0.1% FA, flow rate: 30 mL/min) to give N-(4-amino-1H-pyrazolo[4,3-c]pyridin-7-yl)-2-oxo-2-[rac-(2R,5S)-2-(5-chloro-3-pyridyl)-5-methyl-1-piperidyl]acetamide (19 mg, 41.31 μmol, 27.41% yield, HCOOH).

¹H NMR (600 MHz, dmso) δ 0.86-1.04 (m, 3H), 1.27-1.43 (m, 1H), 1.59-1.73 (m, 1H), 1.84-1.98 (m, 1H), 2.02-2.18 (m, 1H), 2.19-2.30 (m, 1H), 2.73-3.00 (m, 1H), 3.77-4.12 (m, 1H), 4.90-5.62 (m, 1H), 6.64-6.83 (m, 2H), 7.50-7.71 (m, 1H), 7.82-7.91 (m, 1H), 8.15-8.42 (m, 1H), 8.50-8.60 (m, 2H), 9.68-10.57 (m, 1H), 12.56-12.79 (m, 1H).

LCMS(ESI): [M+H]⁺ m/z: calcd 414.2; found 414.2; Rt=1.968 min.

### Example 167. N-(4-Amino-1H-pyrazolo[4,3-c]pyridin-7-yl)-2-oxo-2-[(2R,5S)-5-methyl-2-(1-methylpyrazol-3-yl)-1-piperidyl]acetamide (Compound 201)

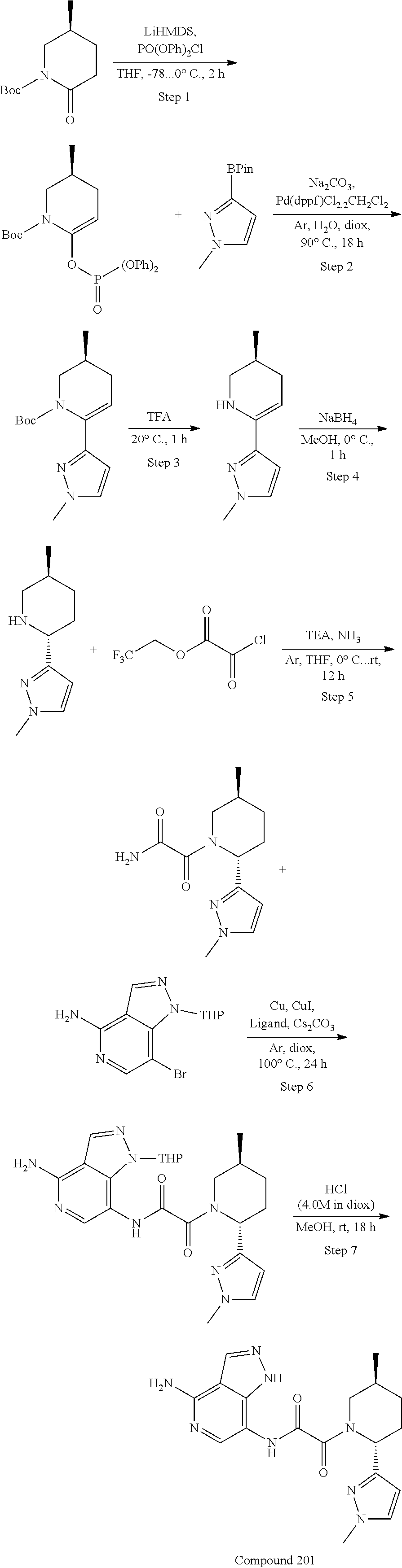

### Step 1: The Synthesis of tert-Butyl rac-(3S)-6-diphenoxyphosphoryloxy-3-methyl-3,4-dihydro-2H-pyridine-1-carboxylate

Lithium bis(trimethylsilyl)amide (245.18 g, 293.05 mmol, 272.42 mL, 20% purity) (1.08 M in THF/ethylbenzene) was added dropwise under argon to a cooled to −78° C. solution of tert-butyl rac-(5S)-5-methyl-2-oxo-piperidine-1-carboxylate (50 g, 234.44 mmol) in THF (500 mL). The resulting solution was stirred at −78° C. for 1.5 hr, then [chloro(phenoxy)phosphoryl]oxybenzene (62.98 g, 234.44 mmol, 48.45 mL) was added dropwise maintaining temperature below −70° C. The reaction mixture was allowed to warm (cooling bath was removed) to 0° C. and then diluted with water (200 ml) and MTBE (500 ml). The organic layer was separated, the aqueous layer was additionally extracted with MTBE (300 ml). The combined organic extracts were washed with 10% aqueous sodium hydroxide solution (2*200 ml), dried over potassium carbonate and concentrated in vacuo. The residue was dissolved in hexane and the resulting cloudy solution was filtered through a pad of silica gel (100 ml). The silica gel pad was additionally washed with hexane (4*250 ml). Combined filtrate was evaporated in vacuo to afford crude tert-butyl rac-(3S)-6-diphenoxyphosphoryloxy-3-methyl-3,4-dihydro-2H-pyridine-1-carboxylate (100 g, 224.50 mmol, 95.76% yield) as light-yellow oil, which was used directly in the next step.

¹H NMR (400 MHZ, CDCl₃) δ (ppm) 0.94 (m, 3H), 1.41 (s, 9H), 1.70 (m, 1H), 1.87 (m, 1H), 2.25 (m, 1H), 2.94 (m, 1H), 3.82 (m, 1H), 5.05 (m, 1H), 7.20 (m, 10H).

### Step 2: The Synthesis of tert-Butyl rac-(3S)-3-methyl-6-(1-methylpyrazol-3-yl)-3,4-dihydro-2H-pyridine-1-carboxylate

A mixture of 1-methyl-3-(4,4,5,5-tetramethyl-1,3,2-dioxaborolan-2-yl) pyrazole (10 g, 48.06 mmol), tert-butyl rac-(3S)-6-diphenoxyphosphoryloxy-3-methyl-3,4-dihydro-2H-pyridine-1-carboxylate (25.19 g, 48.06 mmol), cyclopentyl(diphenyl)phosphane; dichloromethane; dichloropalladium; iron (1.96 g, 2.40 mmol) and sodium carbonate (15.28 g, 144.19 mmol, 6.04 mL) in dioxane (180 mL) and water (60 mL) was stirred at 90° C. under argon atmosphere for 18 hr. The reaction mixture was filtered off and the filtrate was evaporated in vacuo. The residue was purified by silica gel flash chromatography eluting with a 0 to 100 percent chloroform-MeCN gradient to afford product tert-butyl rac-(3S)-3-methyl-6-(1-methylpyrazol-3-yl)-3,4-dihydro-2H-pyridine-1-carboxylate (8 g, 28.84 mmol, 60.01% yield).

LCMS(ESI): [M−tBu]⁺ m/z: calcd 222.2; found 222.2; Rt=1.381 min.

### Step 3: The Synthesis of (3S)-3-Methyl-6-(1-methylpyrazol-3-yl)-2,3,4,5-tetrahydropyridine

The solution of tert-butyl rac-(3S)-3-methyl-6-(1-methylpyrazol-3-yl)-3,4-dihydro-2H-pyridine-1-carboxylate (8 g, 28.84 mmol) in TFA (52.62 g, 461.49 mmol, 35.55 mL) was stirred at 20° C. for 1 hr, and then evaporated in vacuo. Crushed ice (50 g) was added to the residue and the pH was adjusted to 10 with a 10% aqueous solution of potassium carbonate. The resulting mixture was extracted with EA (2*75 mL). The combined organic extracts were dried over sodium sulphate and evaporated in vacuo to afford (3S)-3-methyl-6-(1-methylpyrazol-3-yl)-2,3,4,5-tetrahydropyridine (4.7 g, 26.52 mmol, 91.93% yield) as yellow oil, which was used directly in the next step.

LCMS(ESI): [M+H]⁺ m/z: calcd 178.2; found 178.2; Rt=0.668 min.

### Step 4: The Synthesis of rac-(2R,5S)-5-Methyl-2-(1-methylpyrazol-3-yl)piperidine

Sodium borohydride (1.50 g, 39.78 mmol, 1.40 mL) was added in one portion to a stirred solution of rac-(3S)-3-methyl-6-(1-methylpyrazol-3-yl)-2,3,4,5-tetrahydropyridine (4.7 g, 26.52 mmol) in methanol (100 mL) at 0° C. The resulting mixture was stirred at 0° C. for 1 hr, and then evaporated in vacuo. The residue was diluted with water (100 mL) and extracted with dichloromethane (2*75 mL). The combined organic extracts were dried over sodium sulphate and evaporated in vacuo to afford rac-(2R,5S)-5-methyl-2-(1-methylpyrazol-3-yl)piperidine (4 g, 22.31 mmol, 84.15% yield) as yellow oil, which was used directly in the next step.

LCMS(ESI): [M+H]⁺ m/z: calcd 180.2; found 180.2; Rt=0.558 min.

### Step 5: The Synthesis of 2-Oxo-2-[rac-(2R,5S)-5-methyl-2-(1-methylpyrazol-3-yl)-1-piperidyl]acetamide

To a solution of rac-(2R,5S)-5-methyl-2-(1-methylpyrazol-3-yl)piperidine (3 g, 16.74 mmol) and TEA (3.39 g, 33.47 mmol, 4.67 mL) in THF (50.11 mL) was added 2,2,2-trifluoroethyl 2-chloro-2-oxo-acetate (4.78 g, 25.10 mmol) dropwise at 0° C. under argon. The reaction mixture was then stirred for 12 hr at r.t. Then ammonia was bubbled through for 10 min at 0° C. The reaction mixture was then stirred for 12 hr at r.t. The reaction mixture was filtered off and the filtrate was evaporated in vacuo to give 2-oxo-2-[rac-(2R,5S)-5-methyl-2-(1-methylpyrazol-3-yl)-1-piperidyl]acetamide (4 g, 15.98 mmol, 95.49% yield).

### Step 6: The Synthesis of N-(4-Amino-1-tetrahydropyran-2-yl-pyrazolo[4,3-c]pyridin-7-yl)-2-oxo-2-[rac-(2R,5S)-5-methyl-2-(1-methylpyrazol-3-yl)-1-piperidyl]acetamide

7-Bromo-1-tetrahydropyran-2-yl-pyrazolo[4,3-c]pyridin-4-amine (1 g, 3.37 mmol), 2-oxo-2-[rac-(2R,5S)-5-methyl-2-(1-methylpyrazol-3-yl)-1-piperidyl]acetamide (765.75 mg, 3.06 mmol), Cu (20.61 mg, 324.29 μmol), CuI (174.80 mg, 917.81 μmol, 31.10 μL), Caesium carbonate (1.50 g, 4.59 mmol, 652.93 μL) and (1R,2R)—N1,N2-dimethylcyclohexane-1,2-diamine (130.55 mg, 917.81 μmol) were mixed in dioxane (13 mL), purged with Ar for 5 minutes and then heated in the closed reaction vial at 100° C. for 24 hr. Final mixture was filtered and dioxane was evaporated in vacuo to afford N-(4-amino-1-tetrahydropyran-2-yl-pyrazolo[4,3-c]pyridin-7-yl)-2-oxo-2-[rac-(2R,5S)-5-methyl-2-(1-methylpyrazol-3-yl)-1-piperidyl]acetamide (2 g, crude) as brown solid, which was used directly in the next step.

LCMS(ESI): [M+H]⁺ m/z: calcd 467.2; found 467.2; Rt=0.981 min.

### Step 7: The Synthesis of N-(4-Amino-1H-pyrazolo[4,3-c]pyridin-7-yl)-2-oxo-2-[(2R,5S)-5-methyl-2-(1-methylpyrazol-3-yl)-1-piperidyl]acetamide (Compound 201)

To a solution of N-(4-amino-1-tetrahydropyran-2-yl-pyrazolo[4,3-c]pyridin-7-yl)-2-oxo-2-[rac-(2R,5S)-5-methyl-2-(1-methylpyrazol-3-yl)-1-piperidyl]acetamide (2 g, 1.80 mmol) in methanol (15 mL) was added Hydrogen chloride solution 4.0M in dioxane (16.41 g, 45.01 mmol, 15.63 mL, 10% purity) at 21° C. The resulting mixture was left to stir for 18 hr. The resulting mixture was evaporated to dryness. The residue was purified by RP-HPLC (column: Chromatorex 18 SMB 100-ST 100*19 mm 5 um; 0-0-15% 0-1.3-5.3 min H₂O/MeCN/0.1% FA, flow: 30 mL/min) to give Compound 201_2 N-(4-amino-1H-pyrazolo[4,3-c]pyridin-7-yl)-2-oxo-2-[(2R,5S)-5-methyl-2-(1-methylpyrazol-3-yl)-1-piperidyl]acetamide (198.7 mg, 463.77 μmol, 25.76% yield, HCO₂H).

¹H NMR (600 MHz, dmso) δ 0.78-1.02 (m, 3H), 1.31-1.41 (m, 1H), 1.54-1.74 (m, 1H), 1.84-1.96 (m, 2H), 2.07-2.12 (m, 1H), 2.66-2.93 (m, 1H), 3.67-3.71 (m, 0.6H), 3.76-3.84 (m, 3H), 4.00-4.06 (m, 0.4H), 4.74-5.73 (m, 1H), 6.06-6.27 (m, 1H), 6.79-7.41 (m, 2H), 7.56-7.65 (m, 1H), 7.73-7.87 (m, 1H), 8.18-8.39 (m, 1H), 9.54-10.61 (m, 1H), 12.68-13.50 (m, 1H).

LCMS(ESI): [M+H]⁺ m/z: calcd 383.2; found 383.2; Rt=1.742 min.

### Example 168. N-(4-amino-1H-pyrazolo[4,3-c]pyridin-7-yl)-2-((2R,5S)-2-(3-methoxyphenyl)-5-methylpiperidin-1-yl)-2-oxoacetamide (Compound 195)

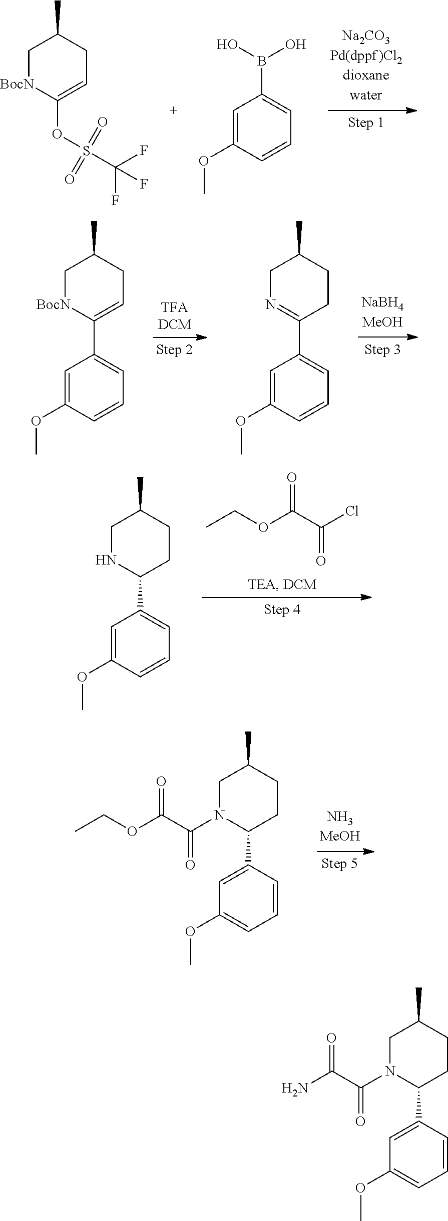

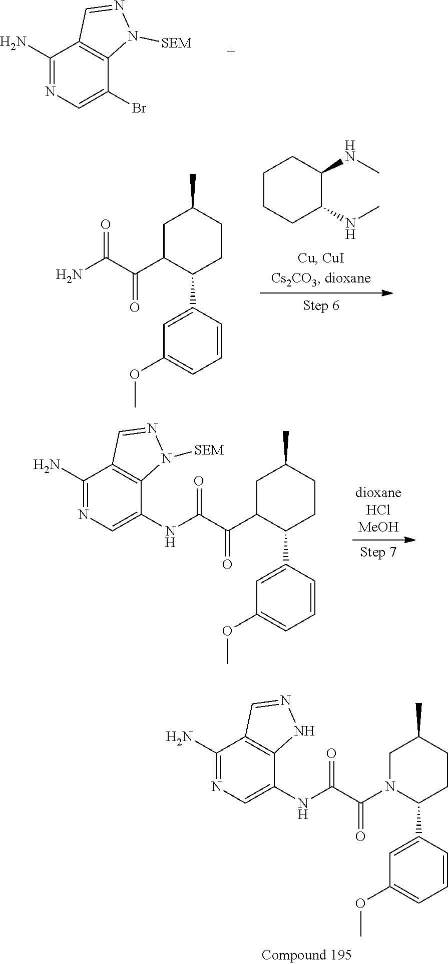

### Step 1: (S)-tert-butyl 6-(3-methoxyphenyl)-3-methyl-3,4-dihydropyridine-1(2H)-carboxylate

tert-butyl (3S)-3-methyl-6-(trifluoromethylsulfonyloxy)-3,4-dihydro-2H-pyridine-1-carboxylate (5.45 g, 15.79 mmol), sodium carbonate (4.19 g, 39.49 mmol, 1.65 mL) and water (30 mL) were added to a solution of (3-methoxyphenyl) boronic acid (2 g, 13.16 mmol) in 1.4-dioxane (160 mL). The resulting mixture was evacuated and then backfilled with argon. This operation was repeated two times, then Pd(dppf)Cl₂-DCM (537.42 mg, 658.09 μmol) was added, and the reaction mixture was stirred under argon at 80° C. for 18 hr, then cooled down and concentrated in vacuo. The residue was diluted with MTBE (60 mL) and stirred for 0.5 hr. After the most of the residue had dissolved, anhydrous sodium sulfate was added, and the resulting mixture was filtered. The filtercake was additionally washed with MTBE (3×20 mL) and discarded. The filtrate was concentrated in vacuo to afford(S)-tert-butyl 6-(3-methoxyphenyl)-3-methyl-3,4-dihydropyridine-1(2H)-carboxylate (3.4 g, 11.21 mmol, 85.14% yield) as a brown gum which was used in the next step without further purification.

LCMS(ESI): [M−Boc+H]⁺ m/z: calcd 204.14; found 204.0; Rt=1.403 min.

### Step 2: (S)-6-(3-methoxyphenyl)-3-methyl-2,3,4,5-tetrahydropyridine

Trifluoroacetic acid (19.17 g, 168.10 mmol, 12.95 mL) was added in one portion to a stirred solution of tert-butyl rac-(3S)-6-(3-methoxyphenyl)-3-methyl-3,4-dihydro-2H-pyridine-1-carboxylate (3.4 g, 11.21 mmol) in dichloromethane (20 mL). The resulting solution was stirred at 25° C. for 0.5 hr, and then concentrated in vacuo. The residue was diluted with water (30 mL). The resulting solution of TFA salt of the product was decanted from dark-brown oily residue, which was additionally rinsed with water (2×10 mL). The combined aqueous solution was basified to pH 11-12 with 10% aqueous sodium carbonate solution and extracted with dichloromethane (2×20 mL). The combined organic extracts were dried over anhydrous sodium sulfate and concentrated in vacuo to afford(S)-6-(3-methoxyphenyl)-3-methyl-2,3,4,5-tetrahydropyridine (2.45 g, 12.05 mmol, 107.55% yield) as a brown gum which was used in the next step without further purification.

LCMS(ESI): [M+H]⁺ m/z: calcd 204.14; found 204.2; Rt=0.679 min.

### Step 3: (2R,5S)-2-(3-methoxyphenyl)-5-methylpiperidine

Sodium borohydride (2.28 g, 60.26 mmol, 2.12 mL) was added in one portion at 0° C. to a stirred solution of (S)-6-(3-methoxyphenyl)-3-methyl-2,3,4,5-tetrahydropyridine (2.45 g, 12.05 mmol) in methanol (50 mL). The reaction mixture was stirred at 0° C. for 1 hr, and then concentrated in vacuo. The residue was diluted with water (10 mL) and extracted with dichloromethane (2×15 mL). The combined organic extracts were dried over anhydrous sodium sulfate and concentrated in vacuo to afford crude (2R,5S)-2-(3-methoxyphenyl)-5-methylpiperidine (1.8 g, 8.77 mmol, 72.75% yield) as a brown gum.

### Step 4: ethyl 2-((2R,5S)-2-(3-methoxyphenyl)-5-methylpiperidin-1-yl)-2-oxoacetate

(2R,5S)-2-(3-methoxyphenyl)-5-methylpiperidine (1.8 g, 8.77 mmol) and TEA (887.22 mg, 8.77 mmol, 1.22 mL) was dissolved in DCM (30 mL), cooled with ice-water bath and then ethyl 2-chloro-2-oxo-acetate (1.20 g, 8.77 mmol, 979.63 μL) in 20 mL of DCM was added dropwise in 10 min. Solution was stirred overnight. The reaction mixture was washed with aqueous solution of NaHCO₃, dried over anhydrous sodium sulphate and evaporated to give ethyl 2-((2R,5S)-2-(3-methoxyphenyl)-5-methylpiperidin-1-yl)-2-oxoacetate (1.95 g, 6.39 mmol, 72.83% yield) as a brown oil.

LCMS(ESI): [M+H]⁺ m/z: calcd 306.17; found 306.2; Rt=1.446 min.

### Step 5:2-((2R,5S)-2-(3-methoxyphenyl)-5-methylpiperidin-1-yl)-2-oxoacetamide

ethyl 2-((2R,5S)-2-(3-methoxyphenyl)-5-methylpiperidin-1-yl)-2-oxoacetate (1.95 g, 6.39 mmol) was dissolved in NH₃/methanol (30 mL) solution (10% by weight) and stirred overnight, then concentrated in vacuo to give 2-((2R,5S)-2-(3-methoxyphenyl)-5-methylpiperidin-1-yl)-2-oxoacetamide (1.35 g, 4.89 mmol, 76.51% yield) as a brown gum.

LCMS(ESI): [M+H]⁺ m/z: calcd 277.16; found 277.2; Rt=1.158 min.

### Step 6: N-[4-amino-1-(2-trimethylsilylethoxymethyl)pyrazolo[4,3-c]pyridin-7-yl]-2-oxo-2-[rac-(2R,5S)-2-(3-methoxyphenyl)-5-methyl-1-piperidyl]acetamide

7-bromo-1-(2-trimethylsilylethoxymethyl)pyrazolo[4,3-c]pyridin-4-amine (310.58 mg, 904.72 μmol), 2-((2R,5S)-2-(3-methoxyphenyl)-5-methylpiperidin-1-yl)-2-oxoacetamide (0.25 g, 904.72 μmol), copper (I) iodide (51.69 mg, 271.41 μmol, 9.20 μL), cesium carbonate (589.55 mg, 1.81 mmol) and (1R,2R)—N1,N2-dimethylcyclohexane-1,2-diamine (57.91 mg, 407.12 μmol) were mixed in dioxane (3 mL) under argon, and then stirred overnight at 100° C. for 12 hr in vial. The reaction mixture was filtered, then filtrate was evaporated. The reaction mixture was filtered, then filtrate was evaporated. The residue was dissolved in 15 mL of DCM, washed with an aqueous ammonium solution, dried over anhydrous sodium sulfate and evaporated to give N-[4-amino-1-(2-trimethylsilylethoxymethyl)pyrazolo[4,3-c]pyridin-7-yl]-2-oxo-2-[rac-(2R,5S)-2-(3-methoxyphenyl)-5-methyl-1-piperidyl]acetamide (385 g, crude).

LCMS(ESI): [M+H]⁺ m/z: calcd 539.28; found 539.2; Rt=1.377 min.

### Step 7: The synthesis of N-(4-amino-1H-pyrazolo[4,3-c]pyridin-7-yl)-2-((2R,5S)-2-(3-methoxyphenyl)-5-methylpiperidin-1-yl)-2-oxoacetamide

N-[4-amino-1-(2-trimethylsilylethoxymethyl)pyrazolo[4,3-c]pyridin-7-yl]-2-oxo-2-[rac-(2R,5S)-2-(3-methoxyphenyl)-5-methyl-1-piperidyl]acetamide (385 g, 714.67 mmol) was dissolved in MeOH (1 mL) and dioxane/HCl (14.29 mol, 1 mL) was added thereto. Then it was stirred at rt for 2 hr. The reaction mixture was evaporated. Crude product was purified by HPLC (Device (Mobile Phase, Column): 2-10 min 40-100% MeOH+NH₃ 30 ml/min) to afford N-(4-amino-1H-pyrazolo[4,3-c]pyridin-7-yl)-2-((2R,5S)-2-(3-methoxyphenyl)-5-methylpiperidin-1-yl)-2-oxoacetamide (0.039 g, 95.48 μmol, 0.0134% yield) as a light-brown solid.

¹H NMR (600 MHz, dmso) δ 1.00-1.07 (m, 3H), 1.27-1.40 (m, 1H), 1.62-1.73 (m, 1H), 1.82-1.96 (m, 1H), 2.00-2.24 (m, 2H), 3.57-4.08 (m, 5H), 5.24-5.60 (m, 1H), 6.80-6.93 (m, 3H), 7.24-7.78 (m, 2H), 7.89-8.00 (m, 1H), 8.64-9.63 (m, 2H), 12.48-13.09 (m, 1H), 13.73-15.07 (m, 1H).

LCMS(ESI): [M+H]⁺ m/z: calcd 409.22; found 409.2; Rt=1.221 min.

### Example 169. The Synthesis of N-(4-Amino-1H-pyrazolo[4,3-c]pyridin-7-yl)-2-oxo-2-[rac-(2R,5S)-2-(1-ethylindazol-5-yl)-5-methyl-1-piperidyl]acetamide (Compound 189)

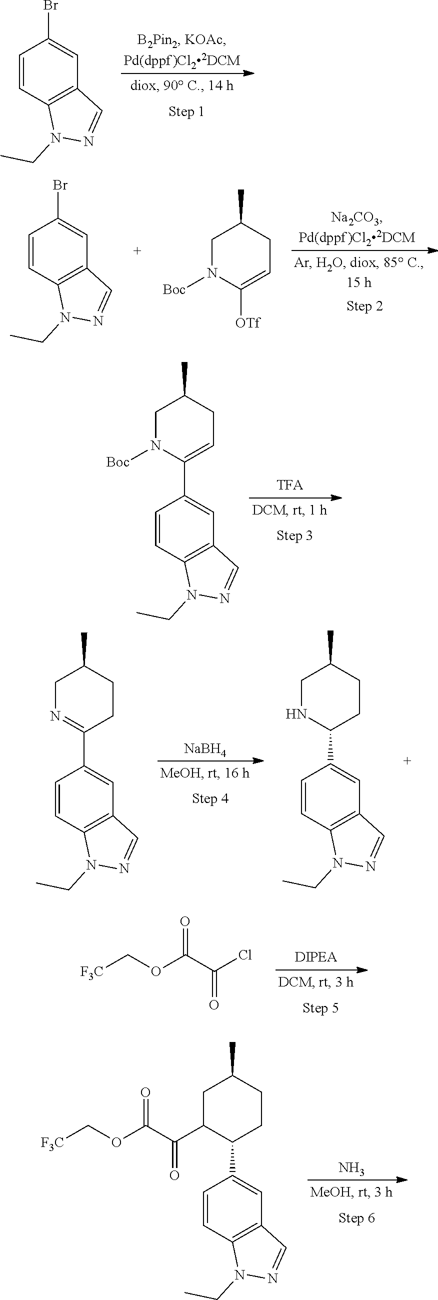

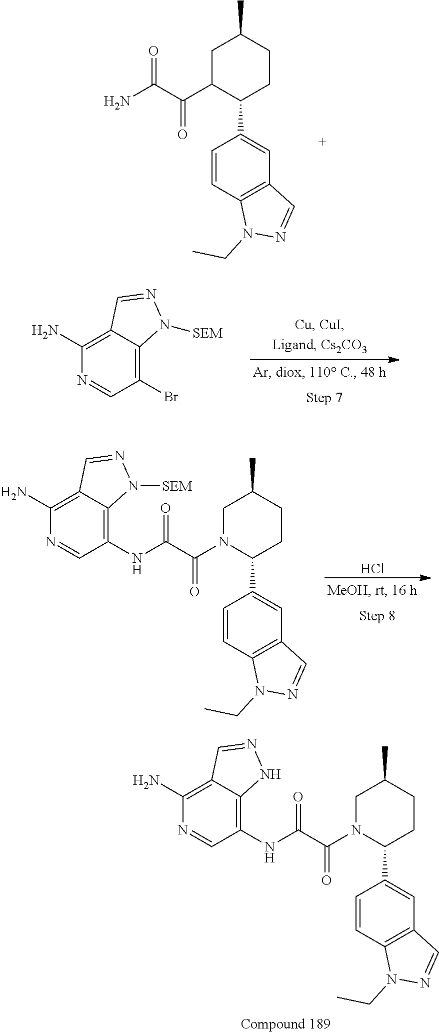

### Step 1: The Synthesis of 1-Ethyl-5-(4,4,5,5-tetramethyl-1,3,2-dioxaborolan-2-yl)indazole

A mixture of 5-bromo-1-ethyl-indazole (6 g, 26.66 mmol), 4,4,5,5-tetramethyl-2-(4,4,5,5-tetramethyl-1,3,2-dioxaborolan-2-yl)-1,3,2-dioxaborolane (7.45 g, 29.32 mmol), Pd(dppf)Cl2 DCM (1.09 g, 1.33 mmol) and Potassium Acetate (7.85 g, 79.97 mmol, 5.00 mL) was stirred in 1,4-dioxane (150.83 mL) at 90° C. for 14 hr. Upon completion, the reaction mixture was diluted with water and extracted with DCM (3×100 mL). Combined organic phases were dried over Na₂SO₄, filtered through a pad of SiO₂ and concentrated under reduced pressure to afford 1-ethyl-5-(4,4,5,5-tetramethyl-1,3,2-dioxaborolan-2-yl)indazole (10 g, crude).

LCMS(ESI): [M+H]⁺ m/z: calcd 273.2; found 273.2; Rt=1.346 min.

### Step 2: The Synthesis of tert-Butyl rac-(3S)-6-(1-ethylindazol-5-yl)-3-methyl-3,4-dihydro-2H-pyridine-1-carboxylate

1-Ethyl-5-(4,4,5,5-tetramethyl-1,3,2-dioxaborolan-2-yl)indazole (6 g, 22.05 mmol), tert-butyl rac-(3S)-3-methyl-6-(trifluoromethylsulfonyloxy)-3,4-dihydro-2H-pyridine-1-carboxylate (12.94 g, 37.48 mmol), Sodium carbonate (7.01 g, 66.14 mmol, 2.77 mL) and Pd(dppf)Cl₂·DCM (900.21 mg, 1.10 mmol) were stirred in a mixture of 1,4-dioxane (75.00 mL) and water (25.00 mL) under inert atmosphere at 85° C. for 15 hr. Upon completion, the reaction mixture was cooled down, diluted with water and extracted with DCM. The organic layer was separated, dried over Na₂SO₄ and concentrated under reduced pressure to afford tert-butyl rac-(3S)-6-(1-ethylindazol-5-yl)-3-methyl-3,4-dihydro-2H-pyridine-1-carboxylate (10.2 g, crude) which was used in the next step without purification.

LCMS(ESI): [M+H]⁺ m/z: calcd 342.2; found 342.2; Rt=1.440 min.

### Step 3: The Synthesis of 1-Ethyl-5-[rac-(3S)-3-methyl-2,3,4,5-tetrahydropyridin-6-yl]indazole

tert-Butyl rac-(3S)-6-(1-ethylindazol-5-yl)-3-methyl-3,4-dihydro-2H-pyridine-1-carboxylate (10.2 g, 29.87 mmol) was dissolved in a mixture of TFA (7.40 g, 64.90 mmol, 5 mL) and DCM (10 mL) and stirred at RT for 1 hr. Upon completion, reaction mixture was concentrated under reduced pressure and the residue was dissolved in DCM, washed with NaHCO₃ solution, dried over Na₂SO₄ and concentrated under reduced pressure to afford 1-ethyl-5-[rac-(3S)-3-methyl-2,3,4,5-tetrahydropyridin-6-yl]indazole (8 g, crude).

LCMS(ESI): [M+H]⁺ m/z: calcd 242.2; found 242.2; Rt=0.910 min.

### Step 4: The Synthesis of 1-Ethyl-5-[rac-(2R,5S)-5-methyl-2-piperidyl]indazole

Sodium Borohydride (2.51 g, 66.30 mmol, 2.34 mL) was added portionwise to a stirred solution of 1-ethyl-5-[rac-(3S)-3-methyl-2,3,4,5-tetrahydropyridin-6-yl]indazole (8 g, 33.15 mmol) in methanol (50 mL). Reaction mixture was stirred at RT overnight and concentrated. The residue was dissolved in DCM, washed with NaHCO₃ solution, dried over Na₂SO₄ and concentrated under reduced pressure to afford 1-ethyl-5-[rac-(2R,5S)-5-methyl-2-piperidyl]indazole (7.6 g, crude).

LCMS(ESI): [M+H]⁺ m/z: calcd 244.2; found 244.2; Rt=0.879 min.

### Step 5: The Synthesis of Ethyl 2-oxo-2-[rac-(2R,5S)-2-(1-ethylindazol-5-yl)-5-methyl-1-piperidyl]acetate

Ethyl 2-chloro-2-oxo-acetate (4.48 g, 32.79 mmol, 3.66 mL) was added dropwise to an ice bath cooled stirred solution of 1-ethyl-5-[rac-(2R,5S)-5-methyl-2-piperidyl]indazole (7.6 g, 31.23 mmol) and DIPEA (5.25 g, 40.60 mmol, 7.07 mL) in DCM (70 mL). Upon completion, the reaction mixture was washed with water. The organic layer was separated, dried over Na₂SO₄ and concentrated under reduced pressure to afford ethyl 2-oxo-2-[rac-(2R,5S)-2-(1-ethylindazol-5-yl)-5-methyl-1-piperidyl]acetate (6.9 g, crude).

LCMS(ESI): [M+H]⁺ m/z: calcd 344.2; found 344.2; Rt=1.180 min.

### Step 6: The Synthesis of 2-Oxo-2-[rac-(2R,5S)-2-(1-ethylindazol-5-yl)-5-methyl-1-piperidyl]acetamide

Ethyl 2-oxo-2-[rac-(2R,5S)-2-(1-ethylindazol-5-yl)-5-methyl-1-piperidyl]acetate (6.9 g, 20.09 mmol) was dissolved in saturated ammonia solution in methanol (50 mL). The reaction mixture was stirred overnight and concentrated under reduced pressure to afford 2-oxo-2-[rac-(2R,5S)-2-(1-ethylindazol-5-yl)-5-methyl-1-piperidyl]acetamide (4.4 g, crude).

LCMS(ESI): [M+H]⁺ m/z: calcd 315.2; found 315.2; Rt=1.010 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-2-(1-ethylindazol-5-yl)-5-methyl-1-piperidyl]acetamide

2-Oxo-2-[rac-(2R,5S)-2-(1-ethylindazol-5-yl)-5-methyl-1-piperidyl]acetamide (500.00 mg, 1.59 mmol), 7-bromo-1-(2-trimethylsilylethoxymethyl)pyrazolo[4,3-c]pyridin-4-amine (451.27 mg, 1.31 mmol), copper (0.09 g, 1.42 mmol), Copper (I) iodide (250.35 mg, 1.31 mmol, 44.55 μL), (1S,2S)—N,N′-Bis-methyl-1,2-cyclohexane-diamine (186.98 mg, 1.31 mmol, 207.29 μL) were mixed in dioxane (15 mL). The reaction mixture was stirred under Ar atmosphere at 100° C. for 48 hr. Upon completion, the reaction mixture was diluted with DCM. Organic layer was washed aq. ammonia solution, dried over Na₂SO₄ and concentrated under reduced pressure. The residue was submitted to HPLC to afford N-[4-amino-1-(2-trimethylsilylethoxymethyl)pyrazolo[4,3-c]pyridin-7-yl]-2-oxo-2-[rac-(2R,5S)-2-(1-ethylindazol-5-yl)-5-methyl-1-piperidyl]acetamide (0.027 g, 46.81 μmol, 3.56% yield) in 3 fractions. All fractions were combined and used in the next step.

LCMS(ESI): [M+H]⁺ m/z: calcd 577.2; found 577.2; Rt=1.105 min.

### Step 8: The Synthesis of N-(4-Amino-1H-pyrazolo[4,3-c]pyridin-7-yl)-2-oxo-2-[rac-(2R,5S)-2-(1-ethylindazol-5-yl)-5-methyl-1-piperidyl]acetamide (Compound 189)

N-[4-Amino-1-(2-trimethylsilylethoxymethyl)pyrazolo[4,3-c]pyridin-7-yl]-2-oxo-2-[rac-(2R,5S)-2-(1-ethylindazol-5-yl)-5-methyl-1-piperidyl]acetamide (0.027 g, 46.81 μmol) was dissolved in HCl/dioxane solution and stirred overnight. Upon completion, the reaction mixture was concentrated under reduced pressure and the residue was submitted to reverse phase HPLC to afford N-(4-amino-1H-pyrazolo[4,3-c]pyridin-7-yl)-2-oxo-2-[rac-(2R,5S)-2-(1-ethylindazol-5-yl)-5-methyl-1-piperidyl]acetamide (0.0036 g, 8.06 μmol, 17.22% yield).

LCMS(ESI): [M+H]⁺ m/z: calcd 447.2; found 447.2; Rt=1.249 min.

### Example 170. N-(4-Amino-2H-pyrazolo[4,3-c]pyridin-7-yl)-2-oxo-2-[rac-(2R,5S)-2-(2-ethoxy-4-pyridyl)-5-methyl-1-piperidyl]acetamide (Compound 187)

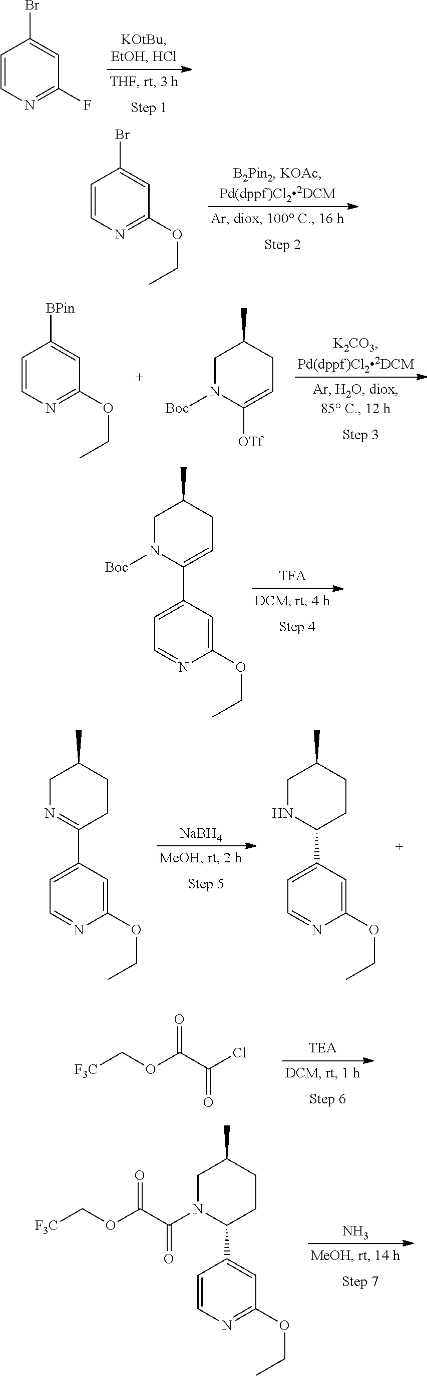

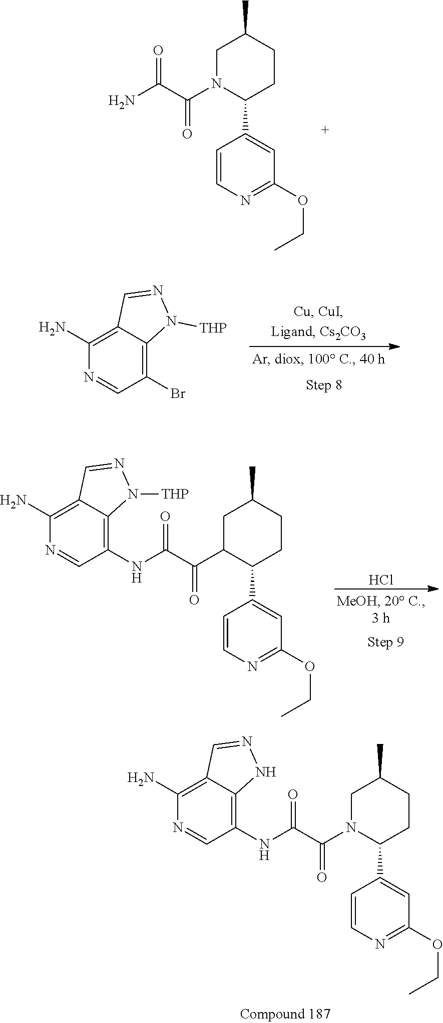

### Step 1: The Synthesis of 4-Bromo-2-ethoxy-pyridine

Potassium tert-butoxide (6.69 g, 59.66 mmol) was added portionwise to the ice-cooled solution of 4-bromo-2-fluoro-pyridine (7 g, 39.78 mmol, 4.09 mL) in Tetrahydrofuran (100 mL) and Ethanol (9.16 g, 198.88 mmol, 11.61 mL). After addition was complete, cooling bath was removed and resulting solution was stirred at 25° C. for 2 hr. Then, volatiles were removed under reduced pressure and residue was partitioned between water (40 mL) and MTBE (70 mL). Organic layer was separated and dried over Na₂SO₄. To the resulting MTBE solution Hydrogen chloride solution 4.0M in dioxane (29.01 g, 79.55 mmol, 28.72 mL, 10% purity) was added dropwise with stirring. Resulting white precipitate was filtered and washed with MTBE. Then it was partitioned between 10% K₂CO₃ solution (40 mL) and MTBE (70 mL). Organic layer was separated, dried over K₂CO₃ and concentrated under reduced pressure, leaving 4-bromo-2-ethoxy-pyridine (5.85 g, 28.95 mmol, 72.79% yield).

¹H NMR (400 MHZ, CDCl₃) δ (ppm) 1.36 (t, 3H), 4.33 (q, 2H), 6.91 (s, 1H), 6.99 (d, 1H), 7.95 (d, 1H).

### Step 2: The Synthesis of 2-Ethoxy-4-(4,4,5,5-tetramethyl-1,3,2-dioxaborolan-2-yl)pyridine

4-Bromo-2-ethoxy-pyridine (5.85 g, 28.95 mmol), 4,4,5,5-tetramethyl-2-(4,4,5,5-tetramethyl-1,3,2-dioxaborolan-2-yl)-1,3,2-dioxaborolane (8.46 g, 33.30 mmol) and Potassium Acetate (5.68 g, 57.91 mmol, 3.62 mL) were mixed together in Dioxane (90 mL). Reaction flask was evacuated and refilled with argon 3 times. Then, PdCl₂*dppf*dcm (472.89 mg, 579.07 μmol) was added under stream of argon. Resulting mixture was stirred at 100° C. for 16 hr. Then, it was diluted with MTBE (90 mL), filtered through a thin layer of silica gel and concentrated under reduced pressure, leaving 2-ethoxy-4-(4,4,5,5-tetramethyl-1,3,2-dioxaborolan-2-yl)pyridine (9.2 g, crude).

¹H NMR (500 MHZ, CDCl₃) δ (ppm) 1.32 (m, 12H), 1.36 (t, 3H), 4.33 (q, 2H), 7.09 (s, 1H), 7.14 (d, 1H), 8.15 (d, 1H).

### Step 3: The Synthesis of tert-Butyl rac-(3S)-6-(2-ethoxy-4-pyridyl)-3-methyl-3,4-dihydro-2H-pyridine-1-carboxylate

Potassium Carbonate (2.04 g, 14.77 mmol, 891.54 μL) was added to a solution of 2-ethoxy-4-(4,4,5,5-tetramethyl-1,3,2-dioxaborolan-2-yl)pyridine (2.3 g, 7.39 mmol) and tert-butyl rac-(3S)-3-methyl-6-(trifluoromethylsulfonyloxy)-3,4-dihydro-2H-pyridine-1-carboxylate (3 g, 8.69 mmol) in Dioxane (30 mL) and Water (15 mL). Reaction flask was evacuated and refilled with argon 3 times. Then, PdCl₂*dppf*dcm (120.64 mg, 147.72 μmol) was added under stream of argon. Resulting mixture was stirred at 85° C. for 12 hr under inert atmosphere. Then, it was concentrated under reduced pressure and residue was extracted with MTBE (50 mL). Obtained solution was filtered through a short pad of silicagel and evaporated under reduced pressure, affording tert-butyl rac-(3S)-6-(2-ethoxy-4-pyridyl)-3-methyl-3,4-dihydro-2H-pyridine-1-carboxylate (2.4 g, crude).

LCMS(ESI): [M+H]⁺ m/z: calcd 319.2; found 319.2; Rt=1.620 min.

### Step 4: The Synthesis of 2-Ethoxy-4-[rac-(3S)-3-Methyl-2,3,4,5-tetrahydropyridin-6-yl]pyridine

Trifluoroacetic acid (8.59 g, 75.37 mmol, 5.81 mL) was added to the solution of tert-butyl rac-(3S)-6-(2-ethoxy-4-pyridyl)-3-methyl-3,4-dihydro-2H-pyridine-1-carboxylate (2.4 g, 7.54 mmol) in Dichloromethane (20 mL). Resulting mixture was stirred at 25° C. for 4 hr. Then, volatiles were removed under reduced pressure and residue was taken up in water (40 mL). Insoluble tar material was filtered off through a cotton wool plug. Clear filtrate was basified with solid K₂CO₃ to pH≈10 and extracted with DCM (2×30 mL). Combined organic layers were dried over K₂CO₃ and concentrated in vacuo, affording 2-ethoxy-4-[rac-(3S)-3-methyl-2,3,4,5-tetrahydropyridin-6-yl]pyridine (1.47 g, 6.72 mmol, 89.10% yield).

LCMS(ESI): [M+H]⁺ m/z: calcd 219.0; found 219.0; Rt=0.777 min.

### Step 5: The Synthesis of 2-Ethoxy-4-[rac-(2R,5S)-5-methyl-2-piperidyl]pyridine

To the stirred solution of 2-ethoxy-4-[rac-(3S)-3-methyl-2,3,4,5-tetrahydropyridin-6-yl]pyridine (1.47 g, 6.73 mmol) in Methanol (30 mL) was added Sodium borohydride (382.15 mg, 10.10 mmol, 355.82 μL) in portions. Resulting mixture was stirred at 25° C. for 2 hr. Then, solvent was removed under reduced pressure and residue was partitioned between water (20 mL) and DCM (40 mL). Organic layer was separated, dried over K₂CO₃ and concentrated in vacuo, affording 2-ethoxy-4-[rac-(2R,5S)-5-methyl-2-piperidyl]pyridine (1.38 g, 6.26 mmol, 93.02% yield).

LCMS(ESI): [M+H]⁺ m/z: calcd 221.2; found 221.2; Rt=0.771 min.

### Step 6: The Synthesis of 2,2,2-Trifluoroethyl 2-oxo-2-[rac-(2R,5S)-2-(2-ethoxy-4-pyridyl)-5-methyl-1-piperidyl]acetate

2-Oxo-2-(2,2,2-trifluoroethoxy) acetic acid (380.73 mg, 2.21 mmol) was added dropwise to the ice-cooled solution of 2-ethoxy-4-[rac-(2R,5S)-5-methyl-2-piperidyl]pyridine (375 mg, 1.70 mmol) and Triethylamine (344.48 mg, 3.40 mmol, 474.49 μL) in Dichloromethane (10 mL). After addition was complete, cooling bath was removed and resulting mixture was allowed to warm up to 25° C. and stirred for 1 hr. Then, 10% aq. NaHCO₃ solution (20 mL) and DCM (20 mL) were added and stirring was continued for 10 minutes. Then, organic layer was separated, dried over Na₂SO₄ and concentrated under reduced pressure, leaving 2,2,2-trifluoroethyl 2-oxo-2-[rac-(2R,5S)-2-(2-ethoxy-4-pyridyl)-5-methyl-1-piperidyl]acetate (605 mg, 1.62 mmol, 94.95% yield).

LCMS(ESI): [M+H]⁺ m/z: calcd 375.2; found 375.2; Rt=1.252 min.

### Step 7: The Synthesis of 2-Oxo-2-[rac-(2R,5S)-2-(2-ethoxy-4-pyridyl)-5-methyl-1-piperidyl]acetamide

2,2,2-Trifluoroethyl 2-oxo-2-[rac-(2R,5S)-2-(2-ethoxy-4-pyridyl)-5-methyl-1-piperidyl]acetate (605 mg, 1.62 mmol) was dissolved in Ammonia (7N in methanol) (3.90 g, 35.45 mmol, 5 mL, 15.5% purity). Resulting mixture was stirred at 25° C. for 14 hr. Then, volatiles were removed under reduced pressure, leaving 2-oxo-2-[rac-(2R,5S)-2-(2-ethoxy-4-pyridyl)-5-methyl-1-piperidyl]acetamide (520 mg, crude).

LCMS(ESI): [M+H]⁺ m/z: calcd 292.2; found 292.2; Rt=0.930 min.

### Step 8: The Synthesis of N-(4-Amino-2-tetrahydropyran-2-yl-pyrazolo[4,3-c]pyridin-7-yl)-2-oxo-2-[rac-(2R,5S)-2-(2-ethoxy-4-pyridyl)-5-methyl-1-piperidyl]acetamide

2-Oxo-2-[rac-(2R,5S)-2-(2-ethoxy-4-pyridyl)-5-methyl-1-piperidyl]acetamide (260 mg, 892.41 μmol), 7-bromo-2-tetrahydropyran-2-yl-pyrazolo[4,3-c]pyridin-4-amine (300 mg, 1.01 mmol), Copper (5.67 mg, 89.24 μmol), Copper (I) iodide (84.98 mg, 446.21 μmol, 15.12 μL), (S,S)-(+)—N,N′-Dimethyl-1,2-cyclohexanediamine (63.47 mg, 446.21 μmol, 70.36 μL) and Cesium carbonate (581.53 mg, 1.78 mmol) were mixed together in Dioxane (5 mL). Reaction flask was purged with argon and resulting mixture was stirred at 100° C. for 40 hr under inert atmosphere. Then, it was diluted with ethyl acetate (15 mL) and filtered. Filtrate was concentrated under reduced pressure and purified by HPLC (40-80% 0-1-5 min H₂O/ACN/NH₄OH flow: 30 mL/min; column: XBridge OBD C18 100×30 mm) affording N-(4-amino-2-tetrahydropyran-2-yl-pyrazolo[4,3-c]pyridin-7-yl)-2-oxo-2-[rac-(2R,5S)-2-(2-ethoxy-4-pyridyl)-5-methyl-1-piperidyl]acetamide (135 mg, 265.97 μmol, 29.80% yield).

LCMS(ESI): [M+2H]⁺ m/z: calcd 509.2; found 509.2; Rt=3.074 min.

### Step 9: The Synthesis of N-(4-Amino-2H-pyrazolo[4,3-c]pyridin-7-yl)-2-oxo-2-[rac-(2R,5S)-2-(2-ethoxy-4-pyridyl)-5-methyl-1-piperidyl]acetamide (Compound 187)

Hydrogen chloride solution 4.0M in dioxane (1.01 g, 2.77 mmol, 1 mL, 10% purity) was added to the solution of N-(4-amino-2-tetrahydropyran-2-yl-pyrazolo[4,3-c]pyridin-7-yl)-2-oxo-2-[rac-(2R,5S)-2-(2-ethoxy-4-pyridyl)-5-methyl-1-piperidyl]acetamide (135 mg, 265.97 μmol) in Methanol (2 mL). Resulting mixture was stirred at 20° C. for 3 hr. Then, solvent was removed under reduced pressure and residue was subjected to HPLC (5-5-30% 0-1-5 min H₂O/ACN/0.2% FA, flow: 30 mL/min; column: Chromatorex 18 SMB100-5T 100×19 mm 5 um), affording N-(4-amino-2H-pyrazolo[4,3-c]pyridin-7-yl)-2-oxo-2-[rac-(2R,5S)-2-(2-ethoxy-4-pyridyl)-5-methyl-1-piperidyl]acetamide (43 mg, 91.59 μmol, 34.44% yield, HCOOH).

¹H NMR (500 MHz, dmso) δ 0.86-1.04 (m, 3H), 1.26-1.42 (m, 4H), 1.53-1.75 (m, 1H), 1.75-1.96 (m, 1H), 1.96-2.24 (m, 2H), 2.69-3.48 (m, 1H), 3.73-4.35 (m, 3H), 4.82-5.61 (m, 1H), 6.40-7.01 (m, 4H), 7.45-7.75 (m, 1H), 8.02-8.29 (m, 3H), 9.57-10.68 (m, 1H), 12.18-13.67 (m, 2H).

LCMS(ESI): [M+H]⁺ m/z: calcd 424.2; found 424.2; Rt=2.506 min.

### Example 171. N-(4-Amino-1H-pyrazolo[4,3-c]pyridin-7-yl)-2-oxo-2-[rac-(2R,5S)-2-(3-fluorophenyl)-5-methyl-1-piperidyl]acetamide (Compound 198)

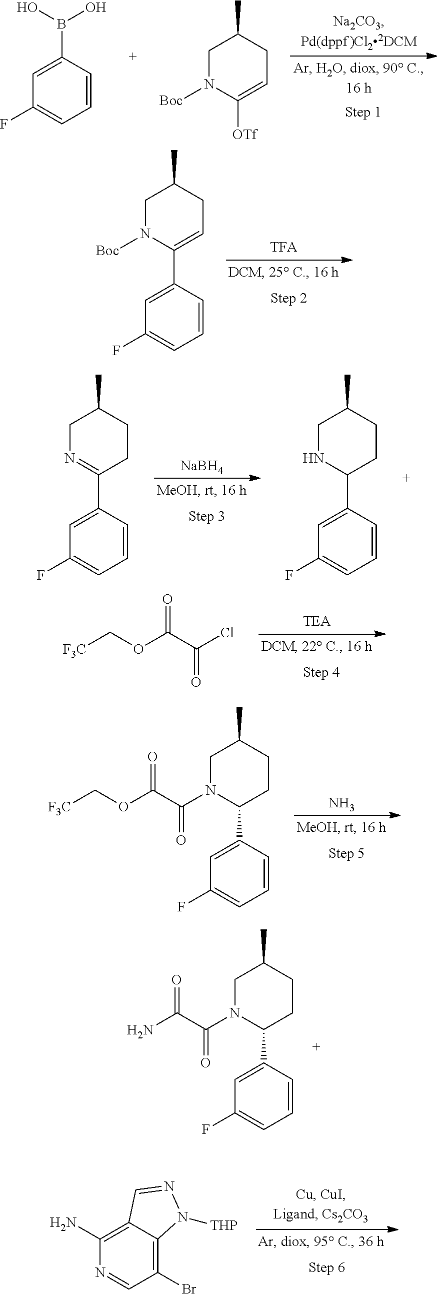

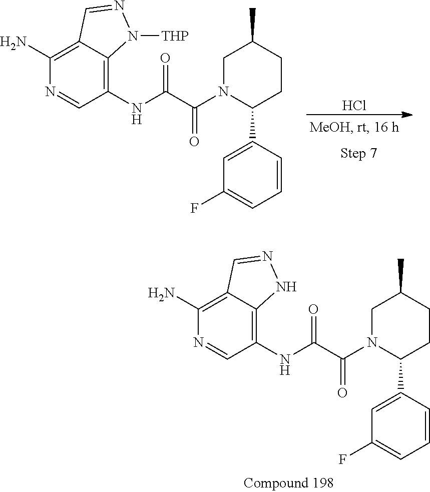

### Step 1: The Synthesis of tert-Butyl rac-(3S)-6-(3-fluorophenyl)-3-methyl-3,4-dihydro-2H-pyridine-1-carboxylate

tert-Butyl rac-(3S)-3-methyl-6-(trifluoromethylsulfonyloxy)-3,4-dihydro-2H-pyridine-1-carboxylate (4.94 g, 14.29 mmol), Sodium carbonate (3.03 g, 28.59 mmol, 1.20 mL) and water (12 mL) were added to a solution (3-fluorophenyl) boronic acid (2 g, 14.29 mmol) in 1,4-dioxane (36 mL). The resulting mixture was evacuated and then backfilled with argon. This operation was repeated two times, then Pd(dppf)Cl₂·DCM (583.65 mg, 714.70 μmol) was added and the reaction mixture was stirred under argon at 90° C. for 16 hr, then cooled down and concentrated in vacuo. The residue was diluted with MTBE (120 mL) and stirred for 0.5 hr. After, the main part of the residue had dissolved, anhydrous sodium sulfate was added, and the resulting mixture was filtered. The filtercake was additionally washed with MTBE (3*20 mL) and discarded. The filtrate was concentrated in vacuo to afford crude tert-butyl rac-(3S)-6-(3-fluorophenyl)-3-methyl-3,4-dihydro-2H-pyridine-1-carboxylate (5 g, crude) as brown oil, which was used directly in the next step.

LCMS(ESI): [M−tBu]⁺ m/z: calcd 236.0; found 236.0; Rt=1.715 min.

### Step 2: The Synthesis of rac-(3S)-6-(3-Fluorophenyl)-3-methyl-2,3,4,5-tetrahydropyridine

Trifluoroacetic acid (14.80 g, 129.80 mmol, 10 mL) was added in one portion to a stirred solution of tert-butyl rac-(3S)-6-(3-fluorophenyl)-3-methyl-3,4-dihydro-2H-pyridine-1-carboxylate (5 g, 17.16 mmol) in dichloromethane (20 mL). The resulting solution was stirred at 25° C. for 16 hr, and then concentrated in vacuo. The residue was diluted with water (50 mL). The resulting solution of TFA salt of the product was decanted from dark-brown oily residue, which was additionally rinsed with water (2*20 mL). The combined aqueous solution was filtered through a cotton pad to remove traces of oily impurities, then basified to pH 11-12 with 10% aqueous sodium carbonate solution and extracted with dichloromethane (2*40 mL). The combined organic extracts were dried over sodium sulfate and concentrated in vacuo to afford rac-(3S)-6-(3-fluorophenyl)-3-methyl-2,3,4,5-tetrahydropyridine (1.12 g, 5.86 mmol, 34.13% yield) which was directly used in the next step.

### Step 3: The Synthesis of rac-(5S)-2-(3-Fluorophenyl)-5-methyl-piperidine

rac-(3S)-6-(3-Fluorophenyl)-3-methyl-2,3,4,5-tetrahydropyridine (1.12 g, 5.86 mmol) was dissolved in MeOH (15 mL) and Sodium Borohydride (664.64 mg, 17.57 mmol, 618.85 μL) was added portionwise. The resulting mixture was stirred overnight. Water (2 mL) was added to the reaction mixture and the resulting mixture was concentrated in vacuo. Water (30 mL) was added to the residue and the resulting mixture was extracted with DCM (2*30 mL). Combined organic layers were dried over Na₂SO₄, filtered and concentrated in vacuo to obtain rac-(5S)-2-(3-fluorophenyl)-5-methyl-piperidine (0.9 g, crude).

LCMS(ESI): [M+2H]⁺ m/z: calcd 195.2; found 195.2; Rt=0.609 min.

### Step 4: The Synthesis of 2,2,2-Trifluoroethyl 2-oxo-2-[rac-(5S)-2-(3-fluorophenyl)-5-methyl-1-piperidyl]acetate

2,2,2-Trifluoroethyl 2-chloro-2-oxo-acetate (379.51 mg, 1.99 mmol) was added dropwise to a solution of rac-(5S)-2-(3-fluorophenyl)-5-methyl-piperidine (0.35 g, 1.81 mmol) and triethylamine (219.91 mg, 2.17 mmol, 302.91 μL) in DCM (10 mL0 at 0° C. After addition was complete, cooling bath was removed and resulting mixture was allowed to warm up to 22° C. and stirred for 16 hr. Then, it was washed with water, dried over Na₂SO₄ and concentrated under reduced pressure, affording 2,2,2-trifluoroethyl 2-oxo-2-[rac-(5S)-2-(3-fluorophenyl)-5-methyl-1-piperidyl]acetate (492 mg, crude) which was used in the next step without further purification.

LCMS(ESI): [M+H]⁺ m/z: calcd 348.0; found 348.0; Rt=1.540 min.

### Step 5: The Synthesis of 2-Oxo-2-[rac-(5S)-2-(3-fluorophenyl)-5-methyl-1-piperidyl]acetamide

A solution of 2,2,2-trifluoroethyl 2-oxo-2-[rac-(5S)-2-(3-fluorophenyl)-5-methyl-1-piperidyl]acetate (492 mg, 1.42 mmol) in Methanol/NH₃ (5N) (5 mL) was stirred at 25° C. for 16 hr. The solvent was evaporated to obtain 2-oxo-2-[rac-(5S)-2-(3-fluorophenyl)-5-methyl-1-piperidyl]acetamide (390 mg, crude).

LCMS(ESI): [M+H]⁺ m/z: calcd 265.2; found 265.2; Rt=0.954 min.

### Step 6: The Synthesis of N-(4-Amino-2-tetrahydropyran-2-yl-pyrazolo[4,3-c]pyridin-7-yl)-2-oxo-2-[rac-(2R,5S)-2-(3-fluorophenyl)-5-methyl-1-piperidyl]acetamide

2-Oxo-2-[rac-(2R,5S)-2-(3-fluorophenyl)-5-methyl-1-piperidyl]acetamide (0.15 g, 567.55 μmol), 7-bromo-2-tetrahydropyran-2-yl-pyrazolo[4,3-c]pyridin-4-amine (177.08 mg, 595.93 μmol), Copper (I) iodide (21.62 mg, 113.51 μmol, 3.85 μL), Cesium carbonate (369.84 mg, 1.14 mmol) and (1R,2R)—N1,N2-dimethylcyclohexane-1,2-diamine (96.87 mg, 681.06 μmol) were mixed in dioxane (4.00 mL) under argon, and then stirred overnight at 95° C. for 36 hr in vial. The reaction mixture was filtered and the filtrate was concentrated in vacuo to give N-(4-amino-2-tetrahydropyran-2-yl-pyrazolo[4,3-c]pyridin-7-yl)-2-oxo-2-[rac-(2R,5S)-2-(3-fluorophenyl)-5-methyl-1-piperidyl]acetamide (0.35 g, crude).

LCMS(ESI): [M+H]⁺ m/z: calcd 481.2; found 481.2; Rt=1.199 min.

### Step 7: The Synthesis of N-(4-Amino-1H-pyrazolo[4,3-c]pyridin-7-yl)-2-oxo-2-[rac-(2R,5S)-2-(3-fluorophenyl)-5-methyl-1-piperidyl]acetamide (Compound 198)

To a solution of N-(4-amino-2-tetrahydropyran-2-yl-pyrazolo[4,3-c]pyridin-7-yl)-2-oxo-2-[rac-(2R,5S)-2-(3-fluorophenyl)-5-methyl-1-piperidyl]acetamide (0.35 g, 371.46 μmol) in MeOH (4.00 mL) was added Hydrogen chloride solution 4.0M in dioxane (800.00 mg, 21.94 mmol, 1.00 mL) at 25° C. The resulting mixture was left to stirred for 16 hr. The resulting mixture was evaporated to dryness and then submitted to reverse phase HPLC (0-2-10 min 3-10-55 H₂O/ACN/0.1FA; flow 30 mL/min ((loading pump 4 mL ACN); column: Chromatorex C18 SMB100-5T 100*19 mm, 5 microM) to afford N-(4-amino-1H-pyrazolo[4,3-c]pyridin-7-yl)-2-oxo-2-[rac-(2R,5S)-2-(3-fluorophenyl)-5-methyl-1-piperidyl]acetamide (32 mg, 72.33 μmol, 19.47% yield, HCOOH).

¹H NMR (600 MHz, dmso) δ 0.85-1.08 (m, 3H), 1.25-1.48 (m, 1H), 1.57-1.75 (m, 1H), 1.82-1.95 (m, 1H), 1.95-2.38 (m, 2H), 2.64-3.13 (m, 1H), 3.70-4.19 (m, 1H), 4.87-5.82 (m, 1H), 6.60-6.92 (m, 2H), 6.91-7.16 (m, 2H), 7.16-7.25 (m, 1H), 7.34-7.51 (m, 1H), 7.51-7.75 (m, 1H), 8.13-8.25 (m, 1H), 9.59-10.68 (m, 1H), 12.32-13.52 (m, 1H).

LCMS(ESI): [M+H]⁺ m/z: calcd 397.2; found 397.2; Rt=1.085 min.

### Example 172. N-(4-amino-1,3-dihydrofuro[3,4-c]pyridin-7-yl)-2-oxo-2-[rac-(2R,5S)-5-methyl-2-[2-(1-methyl-4-piperidyl)-1,3-benzothiazol-5-yl]-1-piperidyl]acetamide (Compound 193)

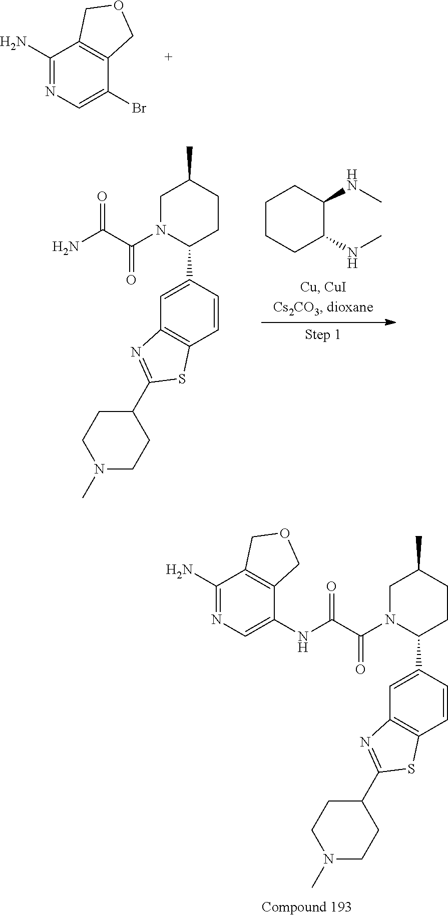

Synthesis of the starting 7-bromo-1,3-dihydrofuro[3,4-c]pyridin-4-amine is described in the document Compound 188.

### Step 1: The synthesis of N-(4-amino-1,3-dihydrofuro[3,4-c]pyridin-7-yl)-2-oxo-2-[rac-(2R,5S)-5-methyl-2-[2-(1-methyl-4-piperidyl)-1,3-benzothiazol-5-yl]-1-piperidyl]acetamide

To a mixture of 7-bromo-1,3-dihydrofuro[3,4-c]pyridin-4-amine (8 mg, 37.20 μmol), 2-((2R,5S)-5-methyl-2-(2-(1-methylpiperidin-4-yl)benzo[d]thiazol-5-yl)piperidin-1-yl)-2-oxoacetamide (18.63 mg, 46.50 μmol), copper (472.83 μg, 7.44 μmol), copper (I) iodide (7.08 mg, 37.20 μmol, 1.26 μL), cesium carbonate (24.24 mg, 74.40 μmol) and rac-(1R,2R)—N1,N2-dimethylcyclohexane-1,2-diamine (7.94 mg, 55.80 μmol), dioxane (4 mL) was added. The resulting mixture was evacuated, refiled with argon three time, heated at 100° C. for 18 hr and cooled. The reaction mixture was subjected to HPLC (Device (Mobile Phase, Column): SYSTEM 20-20-45% 0-2-5 min H₂O/MeOH/0.2% FA, flow: 30 ml/min (loading pump 4 ml/min methanol) target mass 534 column: Chromatorex 18 SMB100-5T 100×19 mm, 5 μm) and repurified (Sample Info: 1 g/L NH₄HCO₃-ACN 20-35% ACN 0.5-6 min 5 ml/min. XBridge C18 7.8×75 mm, 5 μm) to give N-(4-amino-1,3-dihydrofuro[3,4-c]pyridin-7-yl)-2-oxo-2-[rac-(2R,5S)-5-methyl-2-[2-(1-methyl-4-piperidyl)-1,3-benzothiazol-5-yl]-1-piperidyl]acetamide (5 mg, 9.35 μmol, 25.14% yield) as a light-yellow solid.

¹H NMR (600 MHZ, dmso) δ 0.81-1.06 (m, 3H), 1.30-1.40 (m, 1H), 1.64-1.95 (m, 4H), 2.02-2.10 (m, 4H), 2.15-2.33 (m, 4H), 2.74-3.22 (m, 4H), 3.38-4.08 (m, 2H), 4.59-4.95 (m, 4H), 5.14-5.80 (m, 1H), 5.90-6.06 (m, 2H), 7.30-7.45 (m, 1H), 7.70-7.94 (m, 2H), 8.03-8.11 (m, 1H), 10.38-10.65 (m, 1H).

LCMS(ESI): [M−H]⁺ m/z: calcd 533.27; found 533.2; Rt=1.775 min.

### Example 173. N-(4-amino-1,3-dihydrofuro[3,4-c]pyridin-7-yl)-2-oxo-2-[rac-(2R,5S)-5-methyl-2-phenyl-1-piperidyl]acetamide (Compound 188)

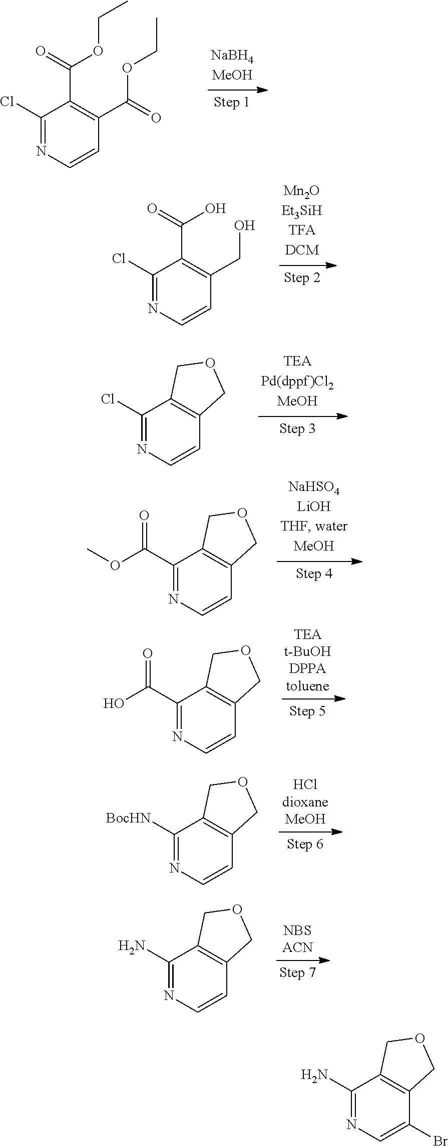

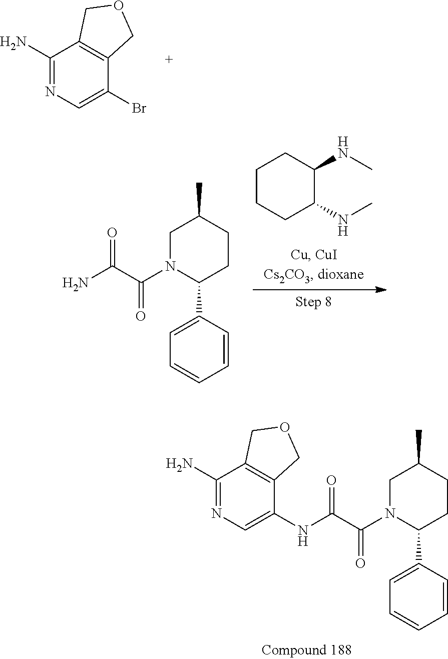

### Step 1: [2-chloro-3-(hydroxymethyl)-4-pyridyl]methanol

To a solution of diethyl 2-chloropyridine-3,4-dicarboxylate (15 g, 58.21 mmol) in ethanol (200 mL), sodium borohydride (8.81 g, 232.86 mmol, 8.20 mL) was added portionwise. The resulting mixture was stirred at 25° C. for 12 hr and pH was adjusted to 5 with formic acid. The solvents was removed in vacuo and the residue was purified by gradient chromatography (cyclohexane-MTBE) to give [2-chloro-3-(hydroxymethyl)-4-pyridyl]methanol (4.1 g, 23.62 mmol, 40.57% yield) as a light-yellow solid.

LCMS(ESI): [M+H]⁺ m/z: calcd 174.03; found 174.0; Rt=0.512 min.

### Step 2:4-chloro-1,3-dihydrofuro[3,4-c]pyridine

To a solution of [2-chloro-3-(hydroxymethyl)-4-pyridyl]methanol (4.1 g, 23.62 mmol) in DCM (150 mL), manganese dioxide (6.84 g, 78.65 mmol), triethylsilane (19.91 g, 171.23 mmol, 27.35 mL) and trifluoroacetic acid (80.79 g, 708.54 mmol, 54.22 mL) was added. The resulting mixture was stirred at 25° C. for 24 hr. The solvent was evaporated and the residue was purified by gradient chromatography (Hexane-MTBE) to give 4-chloro-1,3-dihydrofuro[3,4-c]pyridine (1 g, 6.43 mmol, 27.21% yield) as a light-yellow oil.

LCMS(ESI): [M+H]⁺ m/z: calcd 156.02; found 156.0; Rt=0.760 min.

### Step 3: methyl 1,3-dihydrofuro[3,4-c]pyridine-4-carboxylate

Triethylamine (663.40 mg, 6.56 mmol, 913.77 μL) and Pd(dppf)Cl₂-DCM (446.16 mg, 546.34 μmol) were added to the solution of 4-chloro-1,3-dihydrofuro[3,4-c]pyridine (0.85 g, 5.46 mmol) in MeOH (50 mL). The resulting mixture was stirred in autoclave at 120° C. for 24 hr under CO atmosphere (30 bar). The solvent was evaporated, the residue was taken up with water (10 mL) and extracted with EtOAc (3×15 mL). The combined organic extracts were dried over anhydrous sodium sulfate and evaporated in vacuo to give methyl 1,3-dihydrofuro[3,4-c]pyridine-4-carboxylate (0.9 g, 5.02 mmol, 91.94% yield) as a brown solid.

LCMS(ESI): [M+H]⁺ m/z: calcd 180.07; found 180.0; Rt=0.884 min.

### Step 4:1,3-dihydrofuro[3,4-c]pyridine-4-carboxylic acid

To a solution of methyl 1,3-dihydrofuro[3,4-c]pyridine-4-carboxylate (0.9 g, 5.02 mmol) in THF (20 mL) and MeOH (10 mL) was added lithium hydroxide, monohydrate (350 mg, 8.34 mmol) in water (15 mL). The resulting mixture was stirred at 25° C. for 12 hr. The solvent was evaporated and a solution of sodium bisulfate monohydrate (1.18 g, 8.54 mmol) in water (15 mL). The solvent was evaporated to dryness and residue was washed with THF (2×50 mL). The solvent was evaporated and the residue was taken up with toluene (30 mL) and evaporated to give 1,3-dihydrofuro[3,4-c]pyridine-4-carboxylic acid (0.65 g, 3.94 mmol, 78.36% yield) as a light-yellow solid.

LCMS(ESI): [M+H]⁺ m/z: calcd 166.05; found 166.0; Rt=0.246 min.

### Step 5: tert-butyl (1,3-dihydrofuro[3,4-c]pyridin-4-yl)carbamate

To a solution of 1,3-dihydrofuro[3,4-c]pyridine-4-carboxylic acid (0.65 g, 3.94 mmol), tert-butanol (1.17 g, 15.74 mmol, 1.48 mL) and diphenylphosphoryl azide (1.62 g, 5.90 mmol, 1.27 mL) in toluene (50 mL), triethylamine (1.19 g, 11.81 mmol, 1.65 mL) was added. The resulting mixture was stirred at 80° C. for 18 hr. The solvent was removed in vacuo and the crude reaction mixture was used in the next step.

LCMS(ESI): [M+H]⁺ m/z: calcd 237.13; found 237.0; Rt=0.873 min.

### Step 6:4-chloro-3-methyl-1,3-dihydrofuro[3,4-c]pyridine

A solution of tert-butyl N-(1,3-dihydrofuro[3,4-c]pyridin-4-yl)carbamate (0.9 g, 3.81 mmol) in MeOH (10 mL) and Hydrogen chloride solution 4.0M in dioxane (4 g, 109.71 mmol) as stirred at 25° C. for 4 hr. The solvent was evaporated in vacuo, the residue was taken up with water 50 ml and washed with DCM (3*20 ml). The aqueous layer was evaporated to dryness, the residue was taken up with CHCl3 (50 ml) and NH₃ was bubbled through a solution during 5 min. NH₄Cl-precipitate was filtered off and the solvent was evaporated to give 1,3-dihydrofuro[3,4-c]pyridin-4-amine (0.5 g, 3.67 mmol, 96.41% yield) as a light-yellow solid.

LCMS(ESI): [M+H]⁺ m/z: calcd 137.07; found 137.0; Rt=0.189 min.

### Step 7:7-bromo-1,3-dihydrofuro[3,4-c]pyridin-4-amine

To a solution of 1,3-dihydrofuro[3,4-c]pyridin-4-amine (0.5 g, 3.67 mmol) in ACN (30 mL), NBS (718.99 mg, 4.04 mmol) was added and stirred at 25° C. for 2 hr. The reaction mixture was purified by HPLC (Device (Mobile Phase, Column): SYSTEM 10-10-40% 0-1-5 min H₂O/ACN/0.1% NH₄OH, flow: 30 ml/min (loading pump 4 ml/min acetonitrile) target mass 215 column: XBridge BEH C18 100×19 mm, 5 μm) to give 7-bromo-1,3-dihydrofuro[3,4-c]pyridin-4-amine (59 mg, 274.36 μmol, 7.47% yield) as a light-yellow solid.

LCMS(ESI): [M+H]⁺ m/z: calcd 214.99; found 215.0; Rt=0.510 min.

### Step 8: The synthesis of N-(4-amino-1,3-dihydrofuro[3,4-c]pyridin-7-yl)-2-oxo-2-[rac-(2R,5S)-5-methyl-2-phenyl-1-piperidyl]acetamide

To a mixture of 7-bromo-1,3-dihydrofuro[3,4-c]pyridin-4-amine (0.02 g, 93.00 μmol), 2-((2R,5S)-5-methyl-2-phenylpiperidin-1-yl)-2-oxoacetamide (26.34 mg, 106.95 μmol), copper (1.18 mg, 18.60 μmol), copper (I) iodide (17.71 mg, 93.00 μmol, 3.15 μL), cesium carbonate (60.60 mg, 186.01 μmol) and rac-(1R,2R)—N1,N2-dimethylcyclohexane-1,2-diamine (19.84 mg, 139.50 μmol), dioxane (4 mL) was added. The resulting mixture was evacuated, refiled with argon three time, heated at 90° C. for 18 hr and cooled. The reaction mixture was subjected to HPLC (Device (Mobile Phase, Column): SYSTEM 15-20-45% 0-1-6 min H₂O/ACN/0.1% NH₄OH, flow: 30 ml/min (loading pump 4 ml/min ACN target mass 380 column: XBridge BEH C18 5 μm 130 A) and repurified (Device (Mobile Phase, Column): SYSTEM 30-30-80% 0-1-6 min H₂O/MeOH, flow: 30 ml/min (loading pump 4 ml/min methanol) target mass 380 column: PHENYL SMB100-5 100×19 mm 5 μm) to give N-(4-amino-1,3-dihydrofuro[3,4-c]pyridin-7-yl)-2-oxo-2-[rac-(2R,5S)-5-methyl-2-phenyl-1-piperidyl]acetamide (8 mg, 21.03 μmol, 22.61% yield) as a light-yellow solid.

LCMS(ESI): [M+H]⁺ m/z: calcd 381.22; found 381.2; Rt=2.390 min.

### Example 174. N-(4-amino-1,3-dihydrofuro[3,4-c]pyridin-7-yl)-2-oxo-2-[rac-(2R,5S)-2-(1,3-benzothiazol-5-yl)-5-methyl-1-piperidyl]acetamide (Compound 200)

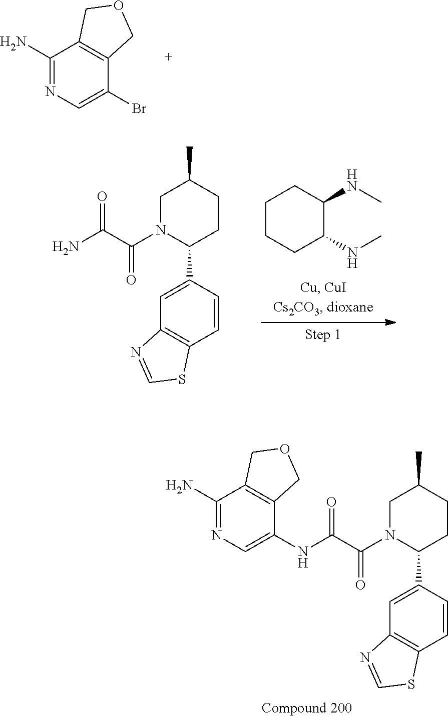

Synthesis of the starting 7-bromo-1,3-dihydrofuro[3,4-c]pyridin-4-amine is described in the document Compound 188

### Step 1: The synthesis of N-(4-amino-1,3-dihydrofuro[3,4-c]pyridin-7-yl)-2-oxo-2-[rac-(2R,5S)-2-(1,3-benzothiazol-5-yl)-5-methyl-1-piperidyl]acetamide

To a mixture of 7-bromo-1,3-dihydrofuro[3,4-c]pyridin-4-amine (20 mg, 93.00 μmol), 2-((2R,5S)-2-(benzo[d]thiazol-5-yl)-5-methylpiperidin-1-yl)-2-oxoacetamide (28.22 mg, 93.00 μmol), copper (1.18 mg, 18.60 μmol), copper (I) iodide (10.63 mg, 55.80 μmol, 1.89 μL), cesium carbonate (33.33 mg, 102.30 μmol) and rac-(1R,2R)—N1,N2-dimethylcyclohexane-1,2-diamine (7.94 mg, 55.80 μmol), dioxane (3 mL) was added. The resulting mixture was evacuated, refiled with argon three time, heated at 85° C. for 72 hr and cooled. The reaction mixture was subjected to HPLC (Device (Mobile Phase, Column): SYSTEM 20-70% 0-5 min H₂O/MeOH/0.1% NH₄OH, flow: 30 ml/min (loading pump 4 ml/min MeOH) target mass 437.52 column: XBridge C18 100×19 mm, 5 μm) to give N-(4-amino-1,3-dihydrofuro[3,4-c]pyridin-7-yl)-2-oxo-2-[rac-(2R,5S)-2-(1,3-benzothiazol-5-yl)-5-methyl-1-piperidyl]acetamide (12 mg, 27.43 μmol, 29.49% yield) as a light-yellow solid.

LCMS(ESI): [M+H]⁺ m/z: calcd 438.18; found 438.2; Rt=2.281 min.

### Example 175. N-(7-amino-1-methyl-1H-pyrazolo[3,4-c]pyridin-4-yl)-2-((2R,5S)-5-methyl-2-phenylpiperidin-1-yl)-2-oxoacetamide (Compound 194)

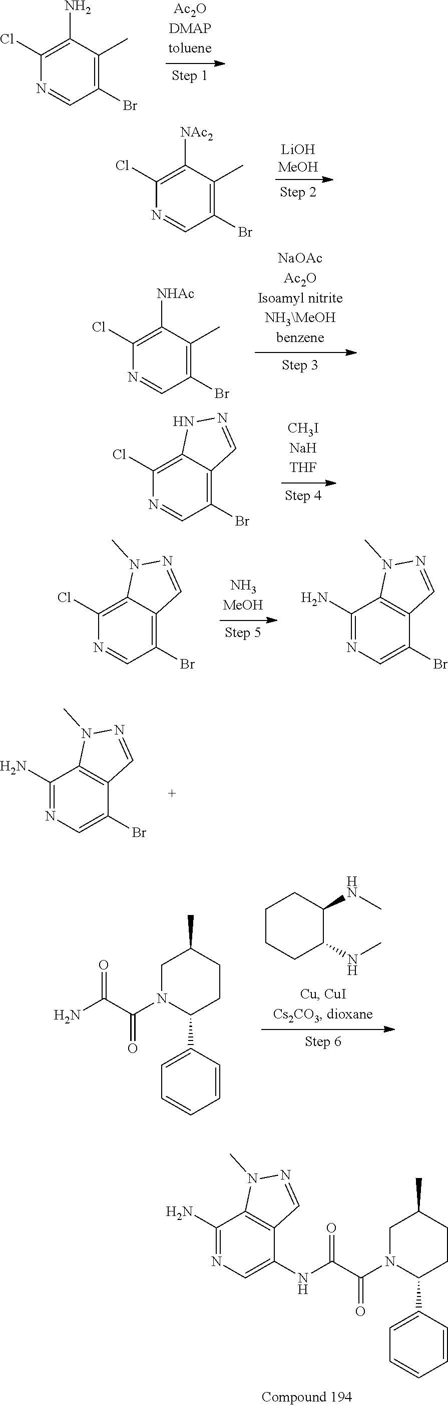

### Step 1: N-acetyl-N-(5-bromo-2-chloro-4-methyl-3-pyridyl)acetamide

5-bromo-2-chloro-4-methyl-pyridin-3-amine (5 g, 22.58 mmol), DMAP (2.76 g, 22.58 mmol) and acetic anhydride (6.91 g, 67.73 mmol, 6.39 mL) were mixed in toluene (99.07 mL) and heated for 48 hr at 100° C. The reaction mixture was washed with HCl (1N), brine, dried and concentrated in vacuo to give N-acetyl-N-(5-bromo-2-chloro-4-methyl-3-pyridyl)acetamide (7.1 g, crude) as a beige solid.

### Step 2: N-(5-bromo-2-chloro-4-methyl-3-pyridyl)acetamide

Lithium hydroxide monohydrate, 98% (4.19 g, 99.82 mmol) was added to a stirred solution of N-acetyl-N-(5-bromo-2-chloro-4-methyl-3-pyridyl)acetamide (6.1 g, 19.96 mmol) in MeOH (25 mL). The reaction mixture was stirred at room temperature for 2 hours and then concentrated in vacuo. The residue was diluted in water and extracted three times with ethyl acetate. The combined organic layers were dried over magnesium sulfate and concentrated in vacuo to give N-(5-bromo-2-chloro-4-methyl-3-pyridyl)acetamide (4.4 g, 16.70 mmol, 83.64% yield) as a pink solid.

LCMS(ESI): [M+H]⁺ m/z: calcd 262.96; found 263.0; Rt=2.002 min.

### Step 3:4-bromo-7-chloro-1H-pyrazolo[3,4-c]pyridine

Potassium Acetate (2.46 g, 25.05 mmol, 1.57 mL) and acetic anhydride (5.11 g, 50.09 mmol, 4.73 mL) were added to a solution of the N-(5-bromo-2-chloro-4-methyl-3-pyridyl)acetamide (4.4 g, 16.70 mmol) in dry benzene (204.65 mL). The reaction mixture was heated at reflux, isoamyl nitrite (3.91 g, 33.39 mmol, 4.47 mL) was added at this temperature and reflux was continued for 16 hr. The insoluble material was then filtered off and the filtrate was vacuum evaporated. The residue was dissolved in a saturated solution of ammonium in methanol (284.36 mg, 16.70 mmol) and the resulting solution was stirred at r.t. for 2 hr and then concentrated in vacuo. The crude product was dissolved in 200 mL of EtOAc and washed with water. The organic phase was dried over anhydrous sodium sulfate and concentrated in vacuo to give 4-bromo-7-chloro-1H-pyrazolo[3,4-c]pyridine (3.6 g, 15.49 mmol, 92.75% yield) as an orange solid.

LCMS(ESI): [M+H]⁺ m/z: calcd 231.93; found 232.0; Rt=2.552 min.

### Step 4:4-bromo-7-chloro-1-methyl-pyrazolo[3,4-c]pyridine

Sodium hydride (941.86 mg, 23.55 mmol, 60% dispersion in mineral oil) was added portionwise to the solution of 4-bromo-7-chloro-1H-pyrazolo[3,4-c]pyridine (3.6 g, 15.49 mmol) in dry THF (48.75 mL). After 30 min iodomethane (2.86 g, 20.13 mmol, 1.25 mL) was added dropwise, then stirred overnight at RT. The reaction mixture was diluted with water and desired compound was extracted with DCM (2×15 mL), dried over anhydrous sodium sulfate and concentrated in vacuo. The residue was subjected to CC-Interchim; 120 g SiO₂ chloroform/ACN with ACN rom 0˜30%, flow rate=85 mL/min, Rv=5-975CV to give 4-bromo-7-chloro-1-methyl-pyrazolo[3,4-c]pyridine (1.2 g, 4.87 mmol, 31.44% yield) as an off-white solid.

### Step 5:4-bromo-1-methyl-pyrazolo[3,4-c]pyridin-7-amine

4-bromo-7-chloro-1-methyl-pyrazolo[3,4-c]pyridine (1.20 g, 4.87 mmol) was dissolved in MeOH (5 mL), ammonia (1.24 g, 73.02 mmol) was bubbled through the solution and this mixture stirred in autoclave at 125° C. for 96 hr. The reaction mixture was brought to rt and evaporated. The residue was purified by CC (Interchim, 40 g SiO₂, ethyl acetate/methanol, flow rate=40 ml/min, RV=7.1-8.8 CV) to give 4-bromo-1-methyl-pyrazolo[3,4-c]pyridin-7-amine (0.2 g, 880.82 μmol, 18.09% yield) as a beige solid.

LCMS(ESI): [M+H]⁺ m/z: calcd 227.0; found 227.0; Rt=0.530 min.

### Step 6: The synthesis of N-(7-amino-1-methyl-1H-pyrazolo[3,4-c]pyridin-4-yl)-2-((2R,5S)-5-methyl-2-phenylpiperidin-1-yl)-2-oxoacetamide

(S,S)-(+)—N,N′-Dimethyl-1,2-cyclohexanediamine (37.59 mg, 264.25 μmol, 41.67 μL), copper (I) iodide (33.55 mg, 176.16 μmol, 5.97 μL), copper (dust) (11.19 mg, 176.16 μmol) and cesium carbonate (229.59 mg, 704.66 μmol, 100.26 μL) were added to a solution of 4-bromo-1-methyl-pyrazolo[3,4-c]pyridin-7-amine (80.00 mg, 352.33 μmol) and 2-oxo-2-[rac-(2R,5S)-5-methyl-2-phenyl-1-piperidyl]acetamide (86.78 mg, 352.33 μmol) in dioxane (4.96 mL). The reaction vessel was purged with argon and the mixture was stirred at 100° C. for 48 hr under an inert atmosphere. Then, it was cooled, diluted with MeOH, and filtered through a short pad of silica gel. The filtrate was concentrated under reduced pressure and the residue was purified by HPLC (1st run: 23-30-70-100% 0-2-6-6.1 min; 30 ml/min water-MeOH+NH₃ (loading pump 4 ml/min MeOH+NH₃); target mass 393 column Xbridge C18 5 μM 19×100 mm. 2nd run: 33-40-70-100% 0-2-7-7.1 min; 30 ml/min water-MeOH (loading pump 4 ml/min MeOH); target mass 393 column Kinetex PFP 5 μM 21.2×100 mm (R)) to give N-(7-amino-1-methyl-1H-pyrazolo[3,4-c]pyridin-4-yl)-2-((2R,5S)-5-methyl-2-phenylpiperidin-1-yl)-2-oxoacetamide (4.7 mg, 11.98 μmol, 3.40% yield) as a colorless gum.

¹H NMR (600 MHz, dmso) δ 1.04 (d, 3H), 1.31-1.39 (m, 1H), 1.63-1.72 (m, 1H), 1.83-1.93 (m, 1H), 2.04-2.27 (m, 2H), 2.72-3.19 (m, 1H), 3.42-4.07 (m, 1H), 4.20-4.28 (m, 3H), 5.06-5.66 (m, 1H), 6.16-6.38 (m, 2H), 7.23-7.30 (m, 1H), 7.31-7.48 (m, 4H), 7.71-7.96 (m, 2H), 10.57-10.84 (m, 1H).

LCMS(ESI): [M+H]⁺ m/z: calcd 393.23; found 393.2; Rt=2.634 min.

### Example 176. 2-oxo-N-[rel-(3R)-4-amino-3-methyl-1,3-dihydrofuro[3,4-c]pyridin-7-yl]-2-[rac-(2R,5S)-5-methyl-2-phenyl-1-piperidyl]acetamide (Compound 192) & 2-oxo-N-[rel-(3S)-4-amino-3-methyl-1,3-dihydrofuro[3,4-c]pyridin-7-yl]-2-[rac-(2R,5S)-5-methyl-2-phenyl-1-piperidyl]acetamide (Compound 204)

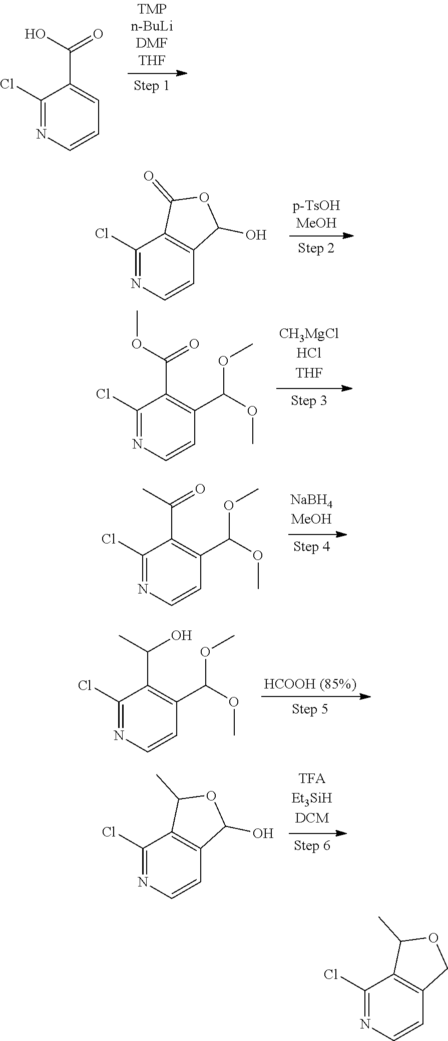

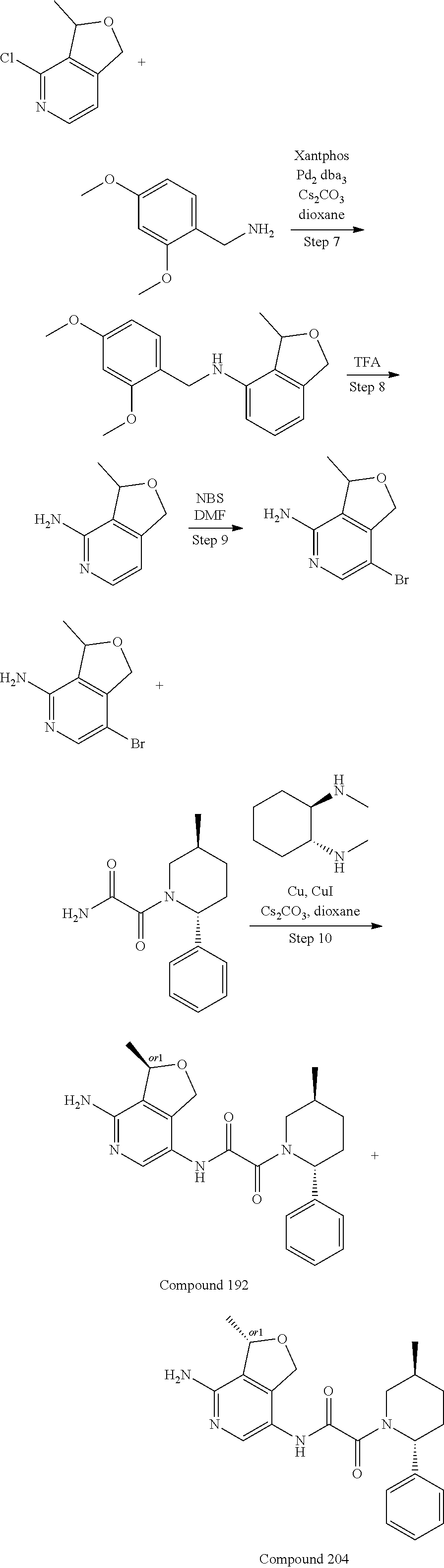

### Step 1:4-chloro-1-hydroxy-1H-furo[3,4-c]pyridin-3-one

2,2,6,6-Tetramethylpiperidine (32.28 g, 228.49 mmol, 38.56 mL) was dissolved in THF (300 mL) and the resulting solution was cooled to −10° C. n-Butyllithium (63.64 g, 228.49 mmol, 92.23 mL, 23% purity) was added dropwise at −10° C., and the resulting mixture was stirred for 10 min. The previous mixture was cooled to −80° C. and 2-chloropyridine-3-carboxylic acid (12 g, 76.16 mmol) was added. The resulting mixture was stirred at −50° C. for 3 hr and then cooled to −80° C. DMF (33.40 g, 456.99 mmol, 35.38 mL) was added and the resulting mixture was stirred for 1.5 hr. The reaction mixture was allowed to warm to room temperature and stirred overnight. Water (200 mL) was added and the resulting mixture was extracted with EtOAc (3×150 mL). The aqueous layer was acidified to pH 2 and the resulting mixture was extracted with EtOAc (3×150 mL). The combined organic layers were dried over anhydrous sodium sulfate and filtered. The filtrate was concentrated in vacuo. The residue was purified by FCC (gradient ACN in chloroform from 8% to 100%) to obtain 4-chloro-1-hydroxy-1H-furo[3,4-c]pyridin-3-one (6.5 g, 35.03 mmol, 45.99% yield) as a yellow solid.

LCMS(ESI): [M+H]⁺ m/z: calcd 186.0; found 186.0; Rt=0.663 min.

### Step 2: methyl 2-chloro-4-(dimethoxymethyl)pyridine-3-carboxylate

4-chloro-1-hydroxy-1H-furo[3,4-c]pyridin-3-one (6.3 g, 33.95 mmol) was dissolved in MeOH (125 mL) and p-Toluenesulfonic acid monohydrate (645.81 mg, 3.40 mmol, 520.81 μL) was added. The resulting mixture was heated to reflux and refluxed overnight. The reaction mixture was concentrated in vacuo and the residue was re-dissolved in EtOAc (150 mL). The resulting solution was washed with aq.NaHCO₃ solution (2×50 mL), dried over anhydrous sodium sulfate and filtered. The filtrate was concentrated in vacuo to obtain methyl 2-chloro-4-(dimethoxymethyl)pyridine-3-carboxylate (7.08 g, 28.80 mmol, 84.83% yield) as a yellow oil.

LCMS(ESI): [M+H]⁺ m/z: calcd 246.06; found 246.2; Rt=1.097 min.

### Step 3:1-[2-chloro-4-(dimethoxymethyl)-3-pyridyl]ethanone

methyl 2-chloro-4-(dimethoxymethyl)pyridine-3-carboxylate (5.64 g, 22.94 mmol) was dissolved in THF (170 mL) and the resulting solution was cooled to 0° C. in an ice/methanol bath under an Ar atmosphere. Methylmagnesium chloride, 3M in THF (5.15 g, 68.81 mmol) was added dropwise and the bath was removed. The resulting mixture was allowed to warm to room temperature and stirred for 30 min. Then the reaction mixture was heated at 70° C. (oil bath) overnight. The reaction mixture was cooled in an ice bath and diluted with Hydrochloric acid, 36% w/w aq. soln. (6.97 g, 68.81 mmol, 6.01 mL, 36% purity) (+40 mL of water) was added dropwise. An organic layer was separated, and the aqueous layer was extracted with EtOAc (2×200 mL). The combined organic layers were washed with brine (150 mL), dried over anhydrous sodium sulfate and filtered. The filtrate was concentrated in vacuo to obtain 1-[2-chloro-4-(dimethoxymethyl)-3-pyridyl]ethanone (4.92 g, 21.41 mmol, 93.36% yield) as a red thick oil which was used in the next step without further purification.

LCMS(ESI): [M+H]⁺ m/z: calcd 230.06; found 230.2; Rt=1.052 min.

### Step 4:1-[2-chloro-4-(dimethoxymethyl)-3-pyridyl]ethanol

1-[2-chloro-4-(dimethoxymethyl)-3-pyridyl]ethanone (4.77 g, 20.76 mmol) was dissolved in MeOH (100 mL) and the resulting mixture was cooled to 0° C. in an ice/methanol bath. Sodium borohydride (2.75 g, 72.66 mmol, 2.56 mL) was added portionwise. After the addition was completed, the reaction mixture was allowed to warm to room temperature and stirred overnight. The reaction mixture was concentrated in vacuo and water (100 mL) was added to the residue. The resulting mixture was extracted with DCM (2×100 mL) and combined organic layers were dried over anhydrous sodium sulfate and filtered. The filtrate was concentrated in vacuo. The residue was re-dissolved in MeOH (100 mL) and the resulting mixture was cooled to 0° C. in an ice/methanol bath. Sodium borohydride (3.93 g, 103.81 mmol, 3.66 mL) was added portionwise. After the addition was completed, the reaction mixture was allowed to warm to room temperature and stirred overnight. The reaction mixture was concentrated in vacuo and water (100 mL) was added to the residue. The resulting mixture was extracted with DCM (2×100 mL) and combined organic layers were dried over anhydrous sodium sulfate and filtered. The filtrate was concentrated in vacuo to obtain 1-[2-chloro-4-(dimethoxymethyl)-3-pyridyl]ethanol (4.57 g, 19.72 mmol, 94.99% yield) as a red thick oil which was used in the next step without further purification.

LCMS(ESI): [M+H]⁺ m/z: calcd 232.08; found 232.0; Rt=1.015 min.

### Step 5:4-chloro-3-methyl-1,3-dihydrofuro[3,4-c]pyridin-1-ol

1-[2-chloro-4-(dimethoxymethyl)-3-pyridyl]ethanol (4.57 g, 19.72 mmol) was dissolved in 85% formic acid (40 mL) and the resulting mixture was stirred overnight. The reaction mixture was concentrated in vacuo. The residue was dissolved in DCM (100 mL) and washed with aq. NaHCO₃ solution (2×45 mL). The organic layer was dried over anhydrous sodium sulfate and filtered. The filtrate was concentrated in vacuo. The residue was purified by FCC (gradient MTBE in hexane from 10% to 50%) to obtain 4-chloro-3-methyl-1,3-dihydrofuro[3,4-c]pyridin-1-ol (948 mg, 5.11 mmol, 25.90% yield) as a white solid.

LCMS(ESI): [M+H]⁺ m/z: calcd 186.03; found 186.0; Rt=0.826 min.

### Step 6:4-chloro-3-methyl-1,3-dihydrofuro[3,4-c]pyridine

4-chloro-3-methyl-1,3-dihydrofuro[3,4-c]pyridin-1-ol (630 mg, 3.39 mmol) was suspended in DCM (10 mL) and Trifluoroacetic acid (1.94 g, 16.97 mmol, 1.30 mL) was added. The resulting mixture was stirred for 5 min and triethylsilane (1.18 g, 10.18 mmol, 1.63 mL) was added dropwise. The resulting mixture was stirred for 3 hr. The reaction mixture was poured into aq.NaHCO₃ solution (2 g in 20 mL of water) and the resulting mixture was extracted with DCM (3×45 mL). The combined organic layers were dried over anhydrous sodium sulfate and filtered. The filtrate was concentrated in vacuo. The residue (753 mg) was combined with crude products of other batches and was and purified by FCC (Hexane-MTBE 3:1) to obtain 4-chloro-3-methyl-1,3-dihydrofuro[3,4-c]pyridine (1.15 g, crude).

LCMS(ESI): [M+H]⁺ m/z: calcd 170.04; found 170.0; Rt=1.059 min.

### Step 7: N-[(2,4-dimethoxyphenyl)methyl]-3-methyl-1,3-dihydrofuro[3,4-c]pyridin-4-amine

4-chloro-3-methyl-1,3-dihydrofuro[3,4-c]pyridine (984 mg, 5.80 mmol), (2,4-dimethoxyphenyl)methanamine (1.26 g, 7.54 mmol), cesium carbonate (3.78 g, 11.60 mmol), 4,5-bis(diphenylphosphino)-9,9-dimethylxanthene (503.54 mg, 870.24 μmol) and tris(dibenzylideneacetone)dipalladium(0) (398.45 mg, 435.12 μmol) were mixed in dioxane (20 mL). The resulting mixture was evacuated and backfilled three times with argon. The resulting mixture was heated at 110° C. overnight. The reaction mixture was cooled and filtered. The filtrate was concentrated in vacuo. The residue was combined with residues of other batches and purified by FCC (hexane-MTBE 3:1) to obtain N-[(2,4-dimethoxyphenyl)methyl]-3-methyl-1,3-dihydrofuro[3,4-c]pyridin-4-amine (472 mg, 1.57 mmol, 27.09% yield) as a reddish gum.

LCMS(ESI): [M+H]⁺ m/z: calcd 301.16; found 301.2; Rt=0.728 min.

### Step 8:3-methyl-1,3-dihydrofuro[3,4-c]pyridin-4-amine

N-[(2,4-dimethoxyphenyl)methyl]-3-methyl-1,3-dihydrofuro[3,4-c]pyridin-4-amine (430 mg, 1.43 mmol) was dissolved in TFA (8.6 mL) and the resulting mixture was stirred for 2 hr. The reaction mixture was concentrated in vacuo and the residue was twice re-evaporated with MeOH to obtain 3-methyl-1,3-dihydrofuro[3,4-c]pyridin-4-amine (300 mg, crude) as a light-brown solid.

LCMS(ESI): [M+H]⁺ m/z: calcd 151.09; found 151.0; Rt=0.418 min.

### Step 9:7-bromo-3-methyl-1,3-dihydrofuro[3,4-c]pyridin-4-amine

3-methyl-1,3-dihydrofuro[3,4-c]pyridin-4-amine (200 mg, 1.33 mmol) was dissolved in DMF (4 mL) and the resulting mixture was cooled to 0° C. in an ice bath. A solution of N-bromosuccinimide (237.03 mg, 1.33 mmol) in DMF (4 mL) was slowly added dropwise at 0° C. After the addition was completed, the reaction mixture was stirred at 0° C. for 1 hr. The reaction mixture was diluted with EtOAc (50 mL) and the resulting mixture was washed with water (2×25 mL), and brine (2×30 mL), dried over anhydrous sodium sulfate and filtered. The filtrate was concentrated in vacuo. The residue was purified by FCC (gradient ACN in CHCl₃ from 10% to 50%) to obtain 7-bromo-3-methyl-1,3-dihydrofuro[3,4-c]pyridin-4-amine (40.3 mg, 175.93 μmol, 13.21% yield) and 111 mg of 2nd fraction, which was re-purified by HPLC (0-2-9 min, 3-10-50% ACN/water+NH₄OH, 30 ml/min (loading pump 4 ml/min ACN), target MI 230, column: XBridge BEH C18 100×19 mm, 5 μM) to obtain 7-bromo-3-methyl-1,3-dihydrofuro[3,4-c]pyridin-4-amine (7.5 mg, 32.74 μmol, 2.46% yield) as a red gum.

LCMS(ESI): [M+H]⁺ m/z: calcd 229.0; found 229.0; Rt=0.606 min.

### Step 10: The synthesis of 2-oxo-N-[rel-(3R)-4-amino-3-methyl-1,3-dihydrofuro[3,4-c]pyridin-7-yl]-2-[rac-(2R,5S)-5-methyl-2-phenyl-1-piperidyl]acetamide & 2-oxo-N-[rel-(3S)-4-amino-3-methyl-1,3-dihydrofuro[3,4-c]pyridin-7-yl]-2-[rac-(2R,5S)-5-methyl-2-phenyl-1-piperidyl]acetamide

To an 8 ml vial 7-bromo-3-methyl-1,3-dihydrofuro[3,4-c]pyridin-4-amine (40.3 mg, 175.93 μmol), 2-((2R,5S)-5-methyl-2-phenylpiperidin-1-yl)-2-oxoacetamide (43.33 mg, 175.93 μmol), copper (559.01 μg, 8.80 μmol), copper (I) iodide (16.75 mg, 87.96 μmol, 2.98 μL), (rac-(1R,2R)—N1,N2-dimethylcyclohexane-1,2-diamine (18.77 mg, 131.94 μmol), cesium carbonate (114.64 mg, 351.85 μmol) and dioxane (1.5 mL) were charged. The resulting mixture was purged with argon for 5 min and the vial was capped and heated at 100° C. for 40 hr. The reaction mixture was cooled to room temperature and filtered. The filtercake was rinsed with MeOH (2.5 mL), and the filtrate was submitted to HPLC and purified (0-2-9 min, 13-20-35% ACN/water+NH₄OH, 30 ml/min (loading pump 4 ml/min ACN), target MI 395, column: XBridge BEH C18 100×19 mm, 5 μM) to obtain 13.6 mg of racemate which was chirally separated (Column: CHIRALPAK AD-H (250×20 mm, 5 mkm)-V, Mobile Phase: MeOH:IPA, 50:50, flow rate: 12 ml/min. Rt (Compound 204)=22.358 min, Rt (Compound 192)=47.991 min) to obtain 2-oxo-N-[rel-(3S)-4-amino-3-methyl-1,3-dihydrofuro[3,4-c]pyridin-7-yl]-2-[rac-(2R,5S)-5-methyl-2-phenyl-1-piperidyl]acetamide (4.27 mg, 10.82 μmol, 12.31% yield) and 2-oxo-N-[rel-(3R)-4-amino-3-methyl-1,3-dihydrofuro[3,4-c]pyridin-7-yl]-2-[rac-(2R,5S)-5-methyl-2-phenyl-1-piperidyl]acetamide (3.76 mg, 9.53 μmol, 10.84% yield) as light-yellow gum.

### Compound 192:

¹H NMR (600 MHz, dmso) δ 0.93-1.04 (m, 3H), 1.24-1.36 (m, 4H), 1.59-1.71 (m, 1H), 1.81-1.93 (m, 1H), 1.96-2.26 (m, 2H), 2.72-3.25 (m, 1H), 3.36-4.01 (m, 1H), 4.52-4.97 (m, 2H), 5.00-5.63 (m, 2H), 5.72-6.25 (m, 2H), 7.10-7.42 (m, 5H), 7.74-7.90 (m, 1H), 10.41 (s, 1H).

LCMS(ESI): [M+H]⁺ m/z: calcd 229.0; found 395.2; Rt=2.587 min.

### Compound 204:

¹H NMR (600 MHz, dmso) δ 0.91-1.04 (m, 3H), 1.26-1.36 (m, 4H), 1.60-1.73 (m, 1H), 1.81-1.94 (m, 1H), 1.98-2.29 (m, 2H), 2.74-3.24 (m, 1H), 3.37-4.04 (m, 1H), 4.52-4.95 (m, 2H), 5.06-5.63 (m, 2H), 5.81-6.26 (m, 2H), 7.21-7.41 (m, 5H), 7.74-7.88 (m, 1H), 10.29-10.50 (m, 1H).

LCMS(ESI): [M+H]⁺ m/z: calcd 229.0; found 395.2; Rt=2.407 min.

### Example 177. N-(7-Amino-1-methyl-pyrazolo[3,4-c]pyridin-4-yl)-2-oxo-2-[(2R,5S)-5-methyl-2-[4-(4-methylpiperazin-1-yl)phenyl]-1-piperidyl]acetamide (Compound 191)

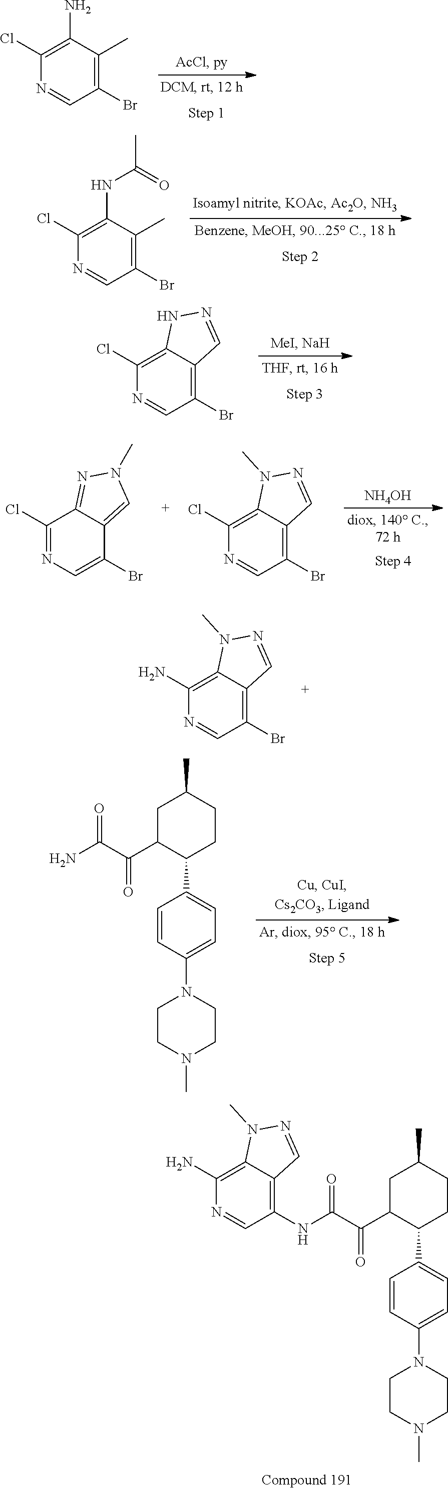

The synthesis of 4-bromo-1-methyl-pyrazolo[3,4-c]pyridin-7-amine (Steps 1-4) was described previously in Compound 194.

### Step 5: The Synthesis of N-(7-Amino-1-methyl-pyrazolo[3,4-c]pyridin-4-yl)-2-oxo-2-[(2R,5S)-5-methyl-2-[4-(4-methylpiperazin-1-yl)phenyl]-1-piperidyl]acetamide (Compound 191)

To a mixture of 4-bromo-1-methyl-pyrazolo[3,4-c]pyridin-7-amine (0.1 g, 440.41 μmol), 2-oxo-2-[(2R,5S)-5-methyl-2-[4-(4-methylpiperazin-1-yl)phenyl]-1-piperidyl]acetamide (174.45 mg, 506.47 μmol), Copper (5.60 mg, 88.08 μmol), Copper (I) iodide (50.33 mg, 264.25 μmol, 8.95 μL), Cesium carbonate (172.19 mg, 528.49 μmol) and (1R,2R)—N1,N2-dimethylcyclohexane-1,2-diamine (37.59 mg, 264.25 μmol), Dioxane (4 mL) was added. The resulting mixture was evacuated, refiled with Argon three time, heated at 95° C. for 18 hr and cooled. The reaction mixture was subjected to HPLC ((Mobile Phase, Column): SYSTEM 20-20-70% 0-1.3-6.3 min H₂O/MeOH/0.1% NH₄OH, flow: 30 mL/min (loading pump 4 mL/min MeOH); column: XBridge BEH C18 5 um 130 A) to give N-(7-amino-1-methyl-pyrazolo[3,4-c]pyridin-4-yl)-2-oxo-2-[(2R,5S)-5-methyl-2-[4-(4-methylpiperazin-1-yl)phenyl]-1-piperidyl]acetamide (46 mg, 93.76 μmol, 21.29% yield).

¹H NMR (600 MHz, dmso) δ 0.91-1.07 (m, 3H), 1.28-1.39 (m, 1H), 1.64-1.74 (m, 1H), 1.81-1.91 (m, 1H), 1.91-2.10 (m, 1H), 2.15-2.21 (m, 4H), 2.40-2.44 (m, 4H), 2.72-2.78 (m, 0.3H), 3.09-3.14 (m, 4H), 3.18-3.25 (m, 0.7H), 3.38-3.99 (m, 1H), 4.22-4.30 (m, 3H), 4.97-5.63 (m, 1H), 6.14-6.24 (m, 2H), 6.89-6.98 (m, 2H), 7.10-7.26 (m, 2H), 7.74-7.97 (m, 2H), 10.53-10.88 (m, 1H).

LCMS(ESI): [M+2H]⁺ m/z: calcd 492.2; found 492.2; Rt=1.634 min.

### Example 178. N-(4-amino-3-methyl-1,3-dihydrofuro[3,4-c]pyridin-7-yl)-2-oxo-2-[(2R,5S)-2-(1,3-benzothiazol-5-yl)-5-methyl-1-piperidyl]acetamide (Compound 196), 2-oxo-N-[rel-(3S)-4-amino-3-methyl-1,3-dihydrofuro[3,4-c]pyridin-7-yl]-2-[(2R,5S)-2-(1,3-benzothiazol-5-yl)-5-methyl-1-piperidyl]acetamide (Compound 287)) and 2-oxo-N-[rel-(3R)-4-amino-3-methyl-1,3-dihydrofuro[3,4-c]pyridin-7-yl]-2-[(2R,5S)-2-(1,3-benzothiazol-5-yl)-5-methyl-1-piperidyl]acetamide (Compound 288)

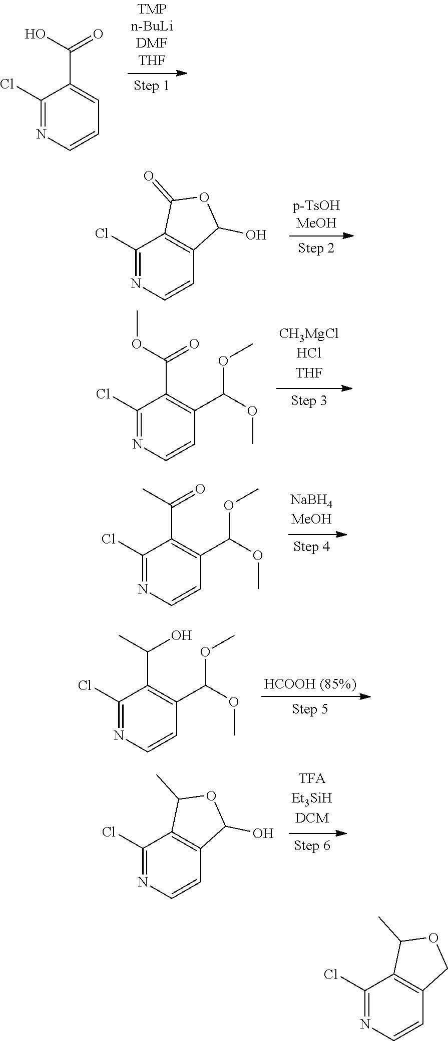

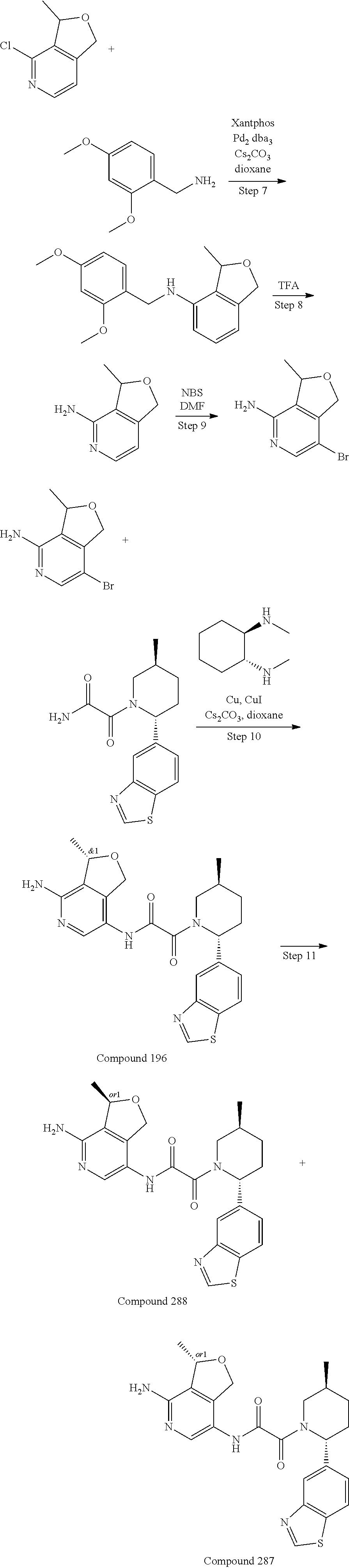

### Step 10: N-(4-amino-3-methyl-1,3-dihydrofuro[3,4-c]pyridin-7-yl)-2-oxo-2-[(2R,5S)-2-(1,3-benzothiazol-5-yl)-5-methyl-1-piperidyl]acetamide (Compound 196)

To an 8 ml vial 2-oxo-2-[rac-(2R,5S)-2-(1,3-benzothiazol-5-yl)-5-methyl-1-piperidyl]acetamide (79.46 mg, 261.92 μmol), 7-bromo-3-methyl-1,3-dihydrofuro[3,4-c]pyridin-4-amine (50 mg, 218.27 μmol), Copper (693.51 μg, 10.91 μmol), Copper (I) iodide (20.78 mg, 109.14 μmol, 3.70 μL), (rac-(1R,2R)—N1,N2-dimethylcyclohexane-1,2-diamine (23.29 mg, 163.70 μmol), Cesium carbonate (142.23 mg, 436.54 μmol) and Dioxane (2 mL) were charged. The resulting mixture was purged with argon for 5 min. the vial was sealed and heated at 80° C. over the weekend. The reaction mixture was diluted with MeOH (2 ml), filtered and the filtrate was submitted to HPLC, and purified (3-10-40%, 0-2-10 H2O/MeCN, flow 30 ml/min ((loading pump 4 ml MeCN), target mass 451, column: XBridge BEH C18 100*19 mm, 5 microM) to obtain N-(4-amino-3-methyl-1,3-dihydrofuro[3,4-c]pyridin-7-yl)-2-oxo-2-[(2R,5S)-2-(1,3-benzothiazol-5-yl)-5-methyl-1-piperidyl]acetamide (32.7 mg, 72.42 μmol, 33.18% yield)

¹H NMR (600 MHz, DMSO-d₆) δ 1.00-1.12 (m, 3H), 1.24-1.36 (m, 4H), 1.65-1.76 (m, 1H), 1.83-1.96 (m, 1H), 2.07-2.25 (m, 1H), 2.28-2.36 (m, 1H), 2.78-3.29 (m, 1H), 3.39-4.10 (m, 1H), 4.52-4.99 (m, 2H), 5.12-5.73 (m, 2H), 5.78-5.94 (m, 2H), 7.38-7.56 (m, 1H), 7.72-7.92 (m, 1H), 7.96-8.10 (m, 1H), 8.18 (d, 1H), 9.40 (s, 1H), 10.37-10.60 (m, 1H).

LCMS(ESI): [M+H]⁺ m/z: calcd 451.2; found 452.2; Rt=2.706 min.

### Step 11:2-oxo-N-[rel-(3S)-4-amino-3-methyl-1,3-dihydrofuro[3,4-c]pyridin-7-yl]-2-[(2R,5S)-2-(1,3-benzothiazol-5-yl)-5-methyl-1-piperidyl]acetamide (Compound 287) and 2-oxo-N-[rel-(3R)-4-amino-3-methyl-1,3-dihydrofuro[3,4-c]pyridin-7-yl]-2-[(2R,5S)-2-(1,3-benzothiazol-5-yl)-5-methyl-1-piperidyl]acetamide (Compound 288)

N-(4-amino-3-methyl-1,3-dihydrofuro[3,4-c]pyridin-7-yl)-2-oxo-2-[(2R,5S)-2-(1,3-benzothiazol-5-yl)-5-methyl-1-piperidyl]acetamide (24 mg, 53.15 μmol) was separated (Column: CHIRALPAK IC (250×21 mm, 5 mkm)-II; Mobile Phase: Hexane (0.1% DEA):IPA:MeOH, 50:25:25 Flow Rate: 14 mL/min; m=0.024 mg, 1 inj., 24 mg/inj., V=2 l, 1.1 hr) to obtain 2-oxo-N-[rel-(3S)-4-amino-3-methyl-1,3-dihydrofuro[3,4-c]pyridin-7-yl]-2-[(2R,5S)-2-(1,3-benzothiazol-5-yl)-5-methyl-1-piperidyl]acetamide (9.71 mg, 21.50 μmol, 40.46% yield) and 2-oxo-N-[rel-(3R)-4-amino-3-methyl-1,3-dihydrofuro[3,4-c]pyridin-7-yl]-2-[(2R,5S)-2-(1,3-benzothiazol-5-yl)-5-methyl-1-piperidyl]acetamide (8.82 mg, 19.53 μmol, 36.75% yield)

RT (Compound 287)=25.206 min.

¹H NMR (600 MHz, dmso) δ 0.93-1.08 (m, 3H), 1.21-1.39 (m, 4H), 1.66-1.77 (m, 1H), 1.84-1.95 (m, 1H), 2.05-2.33 (m, 2H), 2.76-3.24 (m, 1H), 3.40-4.13 (m, 1H), 4.49-4.99 (m, 2H), 5.09-6.23 (m, 4H), 7.32-7.54 (m, 1H), 7.74-7.90 (m, 1H), 7.98-8.08 (m, 1H), 8.13-8.26 (m, 1H), 9.30-9.44 (m, 1H), 10.40-10.57 (m, 1H).

LCMS(ESI): [M+H]⁺ m/z: calcd 451.2; found 452.2; Rt=2.659 min.

RT (Compound 288)=54.349 min.

¹H NMR (600 MHz, dmso) δ 0.94-1.10 (m, 3H), 1.24-1.41 (m, 4H), 1.64-1.82 (m, 1H), 1.82-1.97 (m, 1H), 2.04-2.35 (m, 2H), 2.75-3.26 (m, 1H), 3.39-4.12 (m, 1H), 4.53-5.03 (m, 2H), 5.14-6.28 (m, 4H), 7.37-7.55 (m, 1H), 7.75-7.89 (m, 1H), 7.97-8.08 (m, 1H), 8.14-8.20 (m, 1H), 9.32-9.70 (m, 1H), 10.39-10.54 (m, 1H).

LCMS(ESI): [M+H]⁺ m/z: calcd 451.2; found 452.2; Rt=2.664 min.

### Example 179. N-(7-Amino-1-methyl-pyrazolo[3,4-c]pyridin-4-yl)-2-oxo-2-[(2R,5S)-2-[3-[2-(dimethylamino)ethyl]phenyl]-5-methyl-1-piperidyl]acetamide (Compound 197)

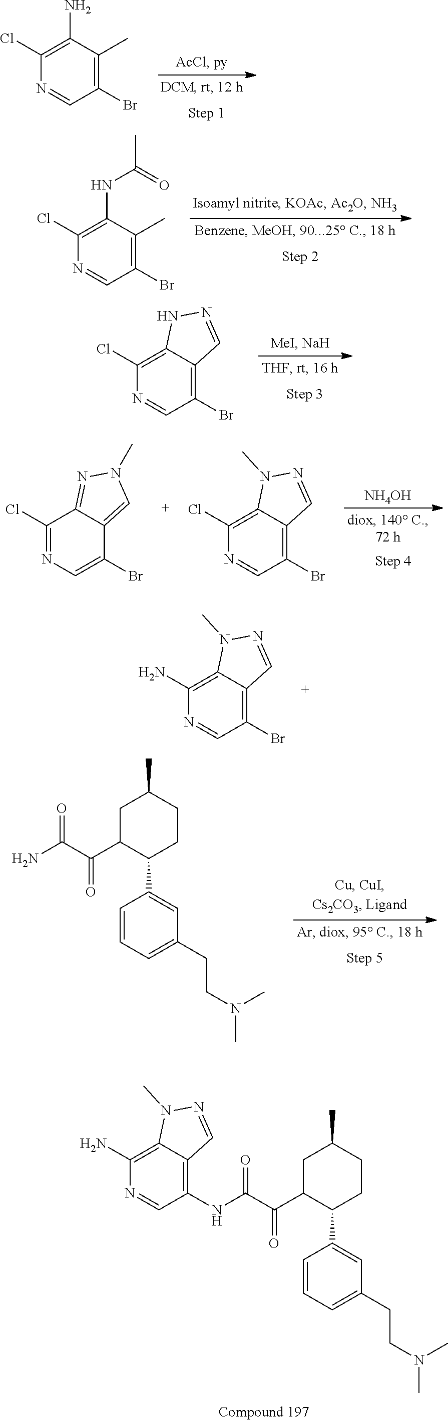

The synthesis of 4-bromo-1-methyl-pyrazolo[3,4-c]pyridin-7-amine (Steps 1-4) was described previously in Compound 194

The synthesis description for 2-oxo-2-[(2R,5S)-2-[3-[2-(dimethylamino)ethyl]phenyl]-5-methyl-1-piperidyl]acetamide is given in Compound 230

### Step 5: The Synthesis of N-(7-Amino-1-methyl-pyrazolo[3,4-c]pyridin-4-yl)-2-oxo-2-[(2R,5S)-2-[3-[2-(dimethylamino)ethyl]phenyl]-5-methyl-1-piperidyl]acetamide (Compound 197)

To a mixture of 4-bromo-1-methyl-pyrazolo[3,4-c]pyridin-7-amine (0.1 g, 440.41 μmol), 2-oxo-2-[(2R,5S)-2-[3-[2-(dimethylamino)ethyl]phenyl]-5-methyl-1-piperidyl]acetamide (160.77 mg, 506.47 μmol), Copper (5.60 mg, 88.08 μmol), Copper (I) iodide (50.33 mg, 264.25 μmol, 8.95 μL), Cesium carbonate (172.19 mg, 528.49 μmol) and (1R,2R)—N1,N2-dimethylcyclohexane-1,2-diamine (37.59 mg, 264.25 μmol), Dioxane (4 mL) was added. The resulting mixture was evacuated, refiled with Argon three time, heated at 100° C. for 18 hr and cooled. The reaction mixture was subjected to HPLC ((Mobile Phase, Column): SYSTEM 25-25-75% 0-1.3-6.3 min H₂O/MeOH/0.1% NH₄OH, flow: 30 mL/min (loading pump 4 mL/min MeOH); column: XBridge BEH C18 5 um 130 A) to give N-(7-amino-1-methyl-pyrazolo[3,4-c]pyridin-4-yl)-2-oxo-2-[(2R,5S)-2-[3-[2-(dimethylamino)ethyl]phenyl]-5-methyl-1-piperidyl]acetamide (48 mg, 103.54 μmol, 23.51% yield).

¹H NMR (600 MHz, dmso) δ 1.01-1.07 (m, 3H), 1.28-1.37 (m, 1H), 1.60-1.70 (m, 1H), 1.82-1.93 (m, 1H), 2.00-2.09 (m, 1H), 2.13-2.16 (m, 6H), 2.19-2.26 (m, 1H), 2.39-2.46 (m, 2H), 2.68-2.75 (m, 2H), 3.14-3.27 (m, 1H), 3.42-4.05 (m, 1H), 4.21-4.29 (m, 3H), 5.07-5.61 (m, 1H), 6.15-6.25 (m, 2H), 7.06-7.15 (m, 2H), 7.20-7.33 (m, 2H), 7.76-7.97 (m, 2H), 10.62-10.80 (m, 1H).

LCMS(ESI): [M+H]⁺ m/z: calcd 464.2; found 464.2; Rt=1.477 min.

### Example 180. N-(7-Amino-1-methyl-pyrazolo[3,4-c]pyridin-4-yl)-2-oxo-2-[(2R,5S)-2-(1,3-benzothiazol-5-yl)-5-methyl-1-piperidyl]acetamide (Compound 199)

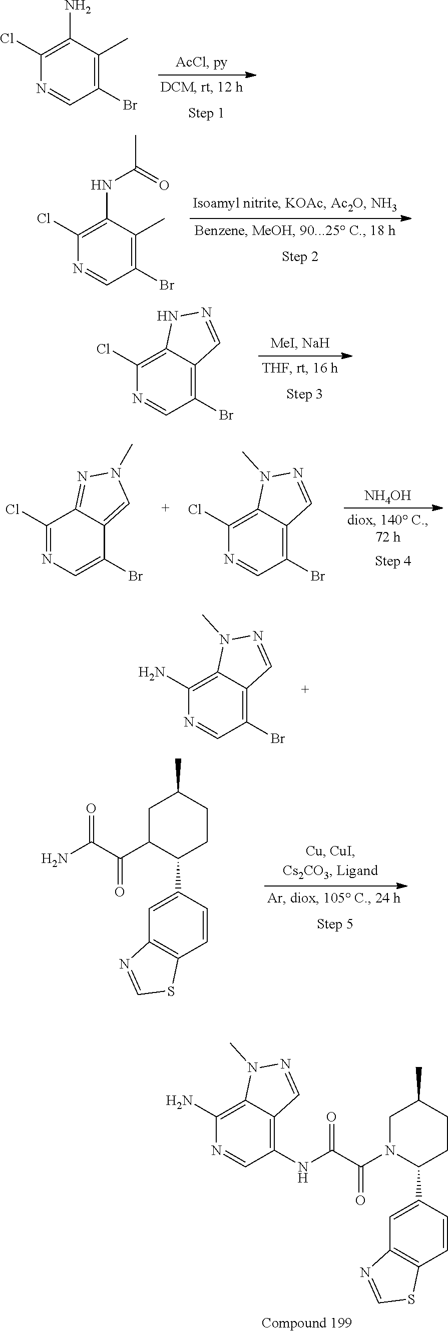

The synthesis of 4-bromo-1-methyl-pyrazolo[3,4-c]pyridin-7-amine (Steps 1-4) was described previously in Compound 194

### Step 5: The Synthesis of N-(7-Amino-1-methyl-pyrazolo[3,4-c]pyridin-4-yl)-2-oxo-2-[(2R,5S)-2-(1,3-benzothiazol-5-yl)-5-methyl-1-piperidyl]acetamide (Compound 199)

A mixture of 2-oxo-2-[(2R,5S)-2-(1,3-benzothiazol-5-yl)-5-methyl-1-piperidyl]acetamide (140 mg, 461.47 μmol), 4-bromo-1-methyl-pyrazolo[3,4-c]pyridin-7-amine (100 mg, 440.41 μmol), copper (5 mg, 78.68 μmol), Copper (I) iodide (70.00 mg, 367.55 μmol, 12.46 μL), caesium carbonate (200.89 mg, 616.57 μmol) and (1R,2R)—N1,N2-dimethylcyclohexane-1,2-diamine (70.00 mg, 492.13 μmol) in 1,4-dioxane (6 mL) was stirred in a sealed vial under argon at 105° C. for 24 hr. The reaction mixture was cooled down and submitted to reverse phase HPLC (column: Chromatorex 18 SMB100-5T 100×19 mm 5 um; mobile phase: 10-10-60% 0-1-5 min H₂O/ACN/0.2% FA; flow rate: 30 mL/min (loading pump 4 mL/min acetonitrile)) to afford Compound 199-N-(7-amino-1-methyl-pyrazolo[3,4-c]pyridin-4-yl)-2-oxo-2-[(2R,5S)-2-(1,3-benzothiazol-5-yl)-5-methyl-1-piperidyl]acetamide (58 mg, 129.02 μmol, 29.30% yield) as light-brown solid.

¹H NMR (600 MHz, DMSO-d₆) δ (ppm) 1.00-1.09 (m, 3H), 1.31-1.45 (m, 1H), 1.69-1.79 (m, 1H), 1.85-1.98 (m, 1H), 2.10-2.24 (m, 1H), 2.26-2.40 (m, 1H), 2.85-3.24 (m, 1H), 3.42-4.10 (m, 1H), 4.24-4.34 (m, 3H), 5.16-5.85 (m, 1H), 7.14 (s, 2H), 7.43-7.60 (m, 1H), 7.85-8.05 (m, 2H), 8.10-8.25 (m, 2H), 9.34-9.46 (m, 1H), 10.87-11.11 (m, 1H), 12.24-13.17 (m, 1H).

LCMS(ESI): [M+H]⁺ m/z: calcd 450.2; found 450.2; Rt=2.154 min.

### Example 181. N-(7-Amino-1-methyl-pyrazolo[3,4-c]pyridin-4-yl)-2-oxo-2-[(2R,5S)-2-[4-[2-(dimethylamino)ethyl]phenyl]-5-methyl-1-piperidyl]acetamide (Compound 202)

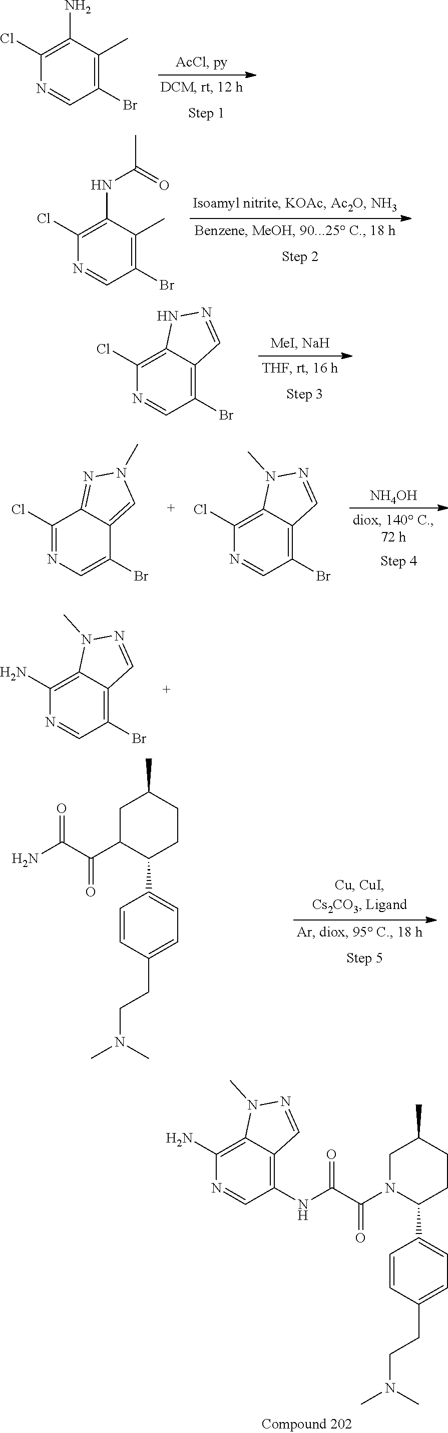

The synthesis of 4-bromo-1-methyl-pyrazolo[3,4-c]pyridin-7-amine (Steps 1-4) was described previously in Compound 194.

The synthesis description for 2-Oxo-2-[(2R,5S)-2-[4-[2-(dimethylamino)ethyl]phenyl]-5-methyl-1-piperidyl]acetamide is given in Compound 263.

### Step 5: The Synthesis of N-(7-Amino-1-methyl-pyrazolo[3,4-c]pyridin-4-yl)-2-oxo-2-[(2R,5S)-2-[4-[2-(dimethylamino)ethyl]phenyl]-5-methyl-1-piperidyl]acetamide (Compound 202)

To a mixture of 4-bromo-1-methyl-pyrazolo[3,4-c]pyridin-7-amine (0.1 g, 440.41 μmol), 2-oxo-2-[(2R,5S)-2-[4-[2-(dimethylamino)ethyl]phenyl]-5-methyl-1-piperidyl]acetamide (160.77 mg, 506.47 μmol), Copper (5.60 mg, 88.08 μmol), Copper (I) iodide (50.33 mg, 264.25 μmol, 8.95 μL), Cesium carbonate (172.19 mg, 528.49 μmol) and (1R,2R)—N1,N2-dimethylcyclohexane-1,2-diamine (37.59 mg, 264.25 μmol), Dioxane was added. The resulting mixture was evacuated, refiled with Argon three time, heated at 95° C. for 18 hr and cooled. The reaction mixture was subjected to HPLC ((Mobile Phase, Column): SYSTEM 30-30-80% 0-1.3-6.3 min H₂O/MeOH/0.1% NH₄OH, flow: 30 mL/min (loading pump 4 mL/min MeOH); column: XBridge BEH C18 5 um 130 A) to give N-(7-amino-1-methyl-pyrazolo[3,4-c]pyridin-4-yl)-2-oxo-2-[(2R,5S)-2-[4-[2-(dimethylamino)ethyl]phenyl]-5-methyl-1-piperidyl]acetamide (37 mg, 79.81 μmol, 18.12% yield).

¹H NMR (600 MHz, dmso) δ 0.93-1.05 (m, 3H), 1.24-1.38 (m, 1H), 1.61-1.71 (m, 1H), 1.80-1.95 (m, 1H), 1.99-2.13 (m, 1H), 2.14-2.16 (m, 6H), 2.17-2.27 (m, 1H), 2.39-2.46 (m, 2H), 2.65-2.71 (m, 2H), 2.77-3.26 (m, 1H), 3.40-4.07 (m, 1H), 4.17-4.34 (m, 3H), 4.98-5.63 (m, 1H), 6.14-6.63 (m, 2H), 7.18-7.32 (m, 4H), 7.67-8.15 (m, 2H), 10.58-10.78 (m, 1H).

LCMS(ESI): [M+H]⁺ m/z: calcd 464.2; found 464.2; Rt=1.455 min.

### Example 182. N-(4-Amino-1H-pyrazolo[4,3-c]pyridin-7-yl)-2-oxo-2-[rac-(2R,5S)-5-methyl-2-(3-thienyl)-1-piperidyl]acetamide (Compound 205)

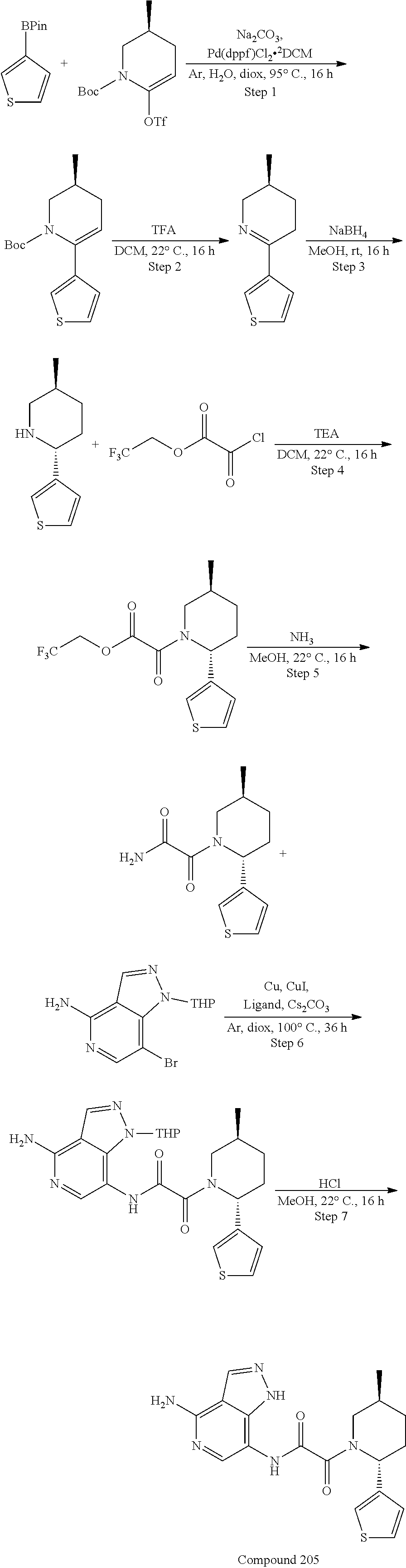

### Step 1: The Synthesis of tert-Butyl rac-(3S)-3-methyl-6-(3-thienyl)-3,4-dihydro-2H-pyridine-1-carboxylate

tert-Butyl rac-(3S)-3-methyl-6-(trifluoromethylsulfonyloxy)-3,4-dihydro-2H-5 pyridine-1-carboxylate (3.62 g, 10.47 mmol), Sodium carbonate (2.22 g, 20.94 mmol, 876.65 μL) and water (12 mL) were added to a solution 4,4,5,5-tetramethyl-2-(3-thienyl)-1,3,2-dioxaborolane (2.2 g, 10.47 mmol) in 1,4-dioxane (36 mL). The resulting mixture was evacuated and then backfilled with argon. This operation was repeated two times, then Pd(dppf)Cl₂·DCM (427.56 mg, 523.56 μmol) was added and the reaction mixture was stirred under argon at 95° C. for 16 hr, then cooled down and concentrated in vacuo. The residue was diluted with MTBE (120 mL) and stirred for 0.5 hr. After, the main part of the residue had dissolved, anhydrous sodium sulfate was added, and the resulting mixture was filtered. The filtercake was additionally washed with MTBE (3*20 mL) and discarded. The filtrate was concentrated in vacuo to afford crude tert-butyl rac-(3S)-3-methyl-6-(3-thienyl)-3,4-dihydro-2H-pyridine-1-carboxylate (3.8 g, crude) as brown oil, which was used directly in the next step.

LCMS(ESI): [M+H]⁺ m/z: calcd 280.2; found 280.2; Rt=1.328 min.

### Step 2: The Synthesis of rac-(3S)-3-Methyl-6-(3-thienyl)-2,3,4,5-tetrahydropyridine

Trifluoroacetic acid (7.45 g, 65.34 mmol, 5 mL) was added in one portion to a stirred solution of tert-butyl rac-(3S)-3-methyl-6-(3-thienyl)-3,4-dihydro-2H-pyridine-1-carboxylate (3.8 g, 13.60 mmol) in dichloromethane (20 mL) The resulting solution was stirred at 22° C. for 16 hr, and then concentrated in vacuo. The residue was diluted with water (50 mL). The resulting solution of TFA salt of the product was decanted from dark-brown oily residue, which was additionally rinsed with water (2*20 mL). The combined aqueous solution was filtered through a cotton pad to remove traces of oily impurities, then basified to pH 11-12 with 10% aqueous sodium carbonate solution and extracted with dichloromethane (2*40 mL). The combined organic extracts were dried over sodium sulfate and concentrated in vacuo to afford rac-(3S)-3-methyl-6-(3-thienyl)-2,3,4,5-tetrahydropyridine (1.11 g, 6.19 mmol, 45.52% yield) which was directly used in the next step.

LCMS(ESI): [M+H]⁺ m/z: calcd 180.0; found 180.0; Rt=0.737 min.

### Step 3: The Synthesis of rac-(5S)-5-Methyl-2-(3-thienyl)piperidine

rac-(3S)-3-Methyl-6-(3-thienyl)-2,3,4,5-tetrahydropyridine (1.11 g, 6.19 mmol) was dissolved in MeOH (20 mL) and Sodium Borohydride (702.66 mg, 18.57 mmol, 654.25 μL) was added portionwise. The resulting mixture was stirred overnight. Water (2 mL) was added to the reaction mixture and the resulting mixture was concentrated in vacuo. Water (30 mL) was added to the residue and the resulting mixture was extracted with DCM (2*30 mL). Combined organic layers were dried over Na₂SO₄, filtered and concentrated in vacuo to obtain rac-(5S)-5-methyl-2-(3-thienyl)piperidine (0.88 g, crude).

LCMS(ESI): [M+H]⁺ m/z: calcd 182.2; found 182.2; Rt=0.326 min.

### Step 4: The Synthesis of 2,2,2-Trifluoroethyl 2-oxo-2-[rac-(2R,5S)-5-methyl-2-(3-thienyl)-1-piperidyl]acetate

2,2,2-Trifluoroethyl 2-chloro-2-oxo-acetate (231.17 mg, 1.21 mmol) was added dropwise to a solution of rac-(2R,5S)-5-methyl-2-(3-thienyl)piperidine (0.2 g, 1.10 mmol) and triethylamine (133.95 mg, 1.32 mmol, 184.51 μL) in DCM (10 mL) at 0° C. After addition was complete, cooling bath was removed and resulting mixture was allowed to warm up to 22° C. and stirred for 16 hr. Then, it was washed with water, dried over Na₂SO₄ and concentrated under reduced pressure, affording 2,2,2-trifluoroethyl 2-oxo-2-[rac-(2R,5S)-5-methyl-2-(3-thienyl)-1-piperidyl]acetate (374 mg, crude) which was used in the next step without further purification.

LCMS(ESI): [M+H]⁺ m/z: calcd 336.0; found 336.0; Rt=1.313 min.

### Step 5: The Synthesis of 2-Oxo-2-[rac-(2R,5S)-5-methyl-2-(3-thienyl)-1-piperidyl]acetamide

A solution of 2,2,2-trifluoroethyl 2-oxo-2-[rac-(2R,5S)-5-methyl-2-(3-thienyl)-1-piperidyl]acetate (374 mg, 1.12 mmol) in Methanol/NH₃ (5N) (5 mL) was stirred at 22° C. for 16 hr. The solvent was evaporated to obtain 2-oxo-2-[rac-(2R,5S)-5-methyl-2-(3-thienyl)-1-piperidyl]acetamide (0.2 g, 792.61 μmol, 71.07% yield).

### Step 6: The Synthesis of N-(4-Amino-2-tetrahydropyran-2-yl-pyrazolo[4,3-c]pyridin-7-yl)-2-oxo-2-[rac-(2R,5S)-5-methyl-2-(3-thienyl)-1-piperidyl]acetamide

2-Oxo-2-[rac-(2R,5S)-5-methyl-2-(3-thienyl)-1-piperidyl]acetamide (0.2 g, 792.61 μmol), 7-bromo-2-tetrahydropyran-2-yl-pyrazolo[4,3-c]pyridin-4-amine (235.52 mg, 792.61 μmol), Copper (I) iodide (30.19 mg, 158.52 μmol, 5.37 μL), Cesium carbonate (516.49 mg, 1.59 mmol) and (1R,2R)—N1,N2-dimethylcyclohexane-1,2-diamine (135.29 mg, 951.13 μmol) were mixed in dioxane (5 mL) under argon, and then stirred overnight at 100° C. for 36 hr in vial. The reaction mixture was filtered and the filtrate was concentrated in vacuo to give N-(4-amino-2-tetrahydropyran-2-yl-pyrazolo[4,3-c]pyridin-7-yl)-2-oxo-2-[rac-(2R,5S)-5-methyl-2-(3-thienyl)-1-piperidyl]acetamide (505 mg, crude).

LCMS(ESI): [M+H]⁺ m/z: calcd 469.2; found 469.2; Rt=0.970 min.

### Step 7: The Synthesis of N-(4-Amino-1H-pyrazolo[4,3-c]pyridin-7-yl)-2-oxo-2-[rac-(2R,5S)-5-methyl-2-(3-thienyl)-1-piperidyl]acetamide (Compound 205)

To a solution of N-(4-amino-2-tetrahydropyran-2-yl-pyrazolo[4,3-c]pyridin-7-yl)-2-oxo-2-[rac-(2R,5S)-5-methyl-2-(3-thienyl)-1-piperidyl]acetamide (505 mg, 474.21 μmol) in MeOH (3 mL) was added Hydrogen chloride solution 4.0M in dioxane (800.00 mg, 21.94 mmol, 1 mL) at 22° C. The resulting mixture was left to stirred for 16 hr. The resulting mixture was evaporated to dryness and then submitted to reverse phase HPLC (SYSTEM 0-2-10 min 28-35-55% H₂O/MeOH/0.1NH₄OH flow 30 mL/min ((loading pump 4 mL MeOH); column: XBridge BEH C18 100*19 mm, 5 microM) to afford N-(4-amino-1H-pyrazolo[4,3-c]pyridin-7-yl)-2-oxo-2-[rac-(2R,5S)-5-methyl-2-(3-thienyl)-1-piperidyl]acetamide (48.9 mg, 127.19 μmol, 26.82% yield).

¹H NMR (600 MHz, dmso) δ 0.72-1.05 (m, 3H), 1.24-1.43 (m, 1H), 1.55-2.06 (m, 3H), 2.10-2.22 (m, 1H), 2.60-3.25 (m, 1H), 3.67-4.08 (m, 1H), 4.91-5.94 (m, 1H), 6.31-6.93 (m, 2H), 6.98-7.21 (m, 1H), 7.24-7.41 (m, 1H), 7.45-7.75 (m, 2H), 8.13-8.22 (m, 1H), 10.93 (s, 1H).

LCMS(ESI): [M+H]⁺ m/z: calcd 385.0; found 385.0; Rt=1.033 min.

### Example 183. N-(4-Amino-1H-pyrazolo[4,3-c]pyridin-7-yl)-2-oxo-2-[rac-(2R,5S)-2-(2,3-dihydrobenzofuran-5-yl)-5-methyl-1-piperidyl]acetamide (Compound 206)

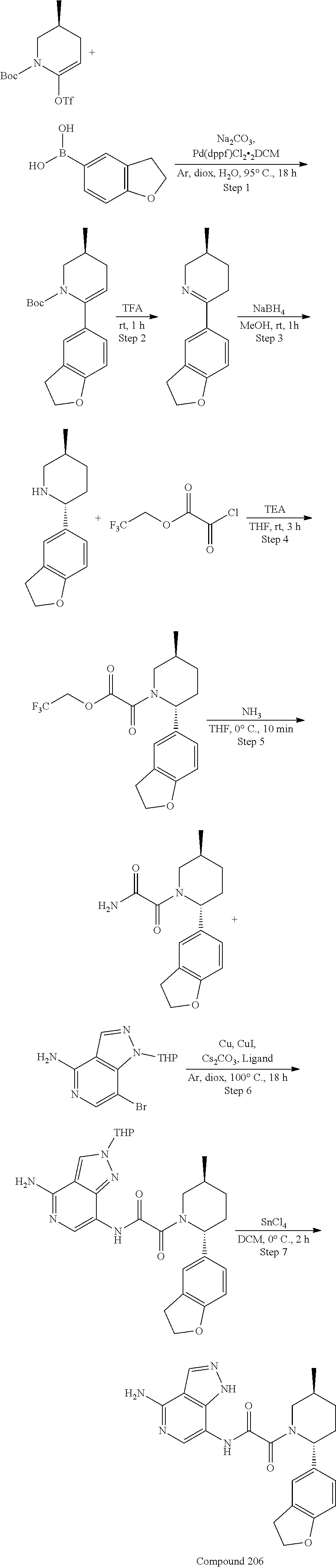

### Step 1: The Synthesis of tert-Butyl rac-(3S)-6-(2,3-dihydrobenzofuran-5-yl)-3-methyl-3,4-dihydro-2H-pyridine-1-carboxylate

To a solution of tert-butyl rac-(3S)-3-methyl-6-(trifluoromethylsulfonyloxy)-3,4-dihydro-2H-pyridine-1-carboxylate (5.05 g, 14.64 mmol), 2,3-dihydrobenzofuran-5-ylboronic acid (2 g, 12.20 mmol), Sodium carbonate (3.88 g, 36.59 mmol, 1.53 mL) in Dioxane (60 mL) and Water (15 mL), Pd(dppf)Cl₂·CH₂Cl₂ (597.66 mg, 731.86 μmol) was added under Argon atmosphere. The resulting mixture was stirred at 95° C. for 18 hr and cooled. The organic layer was separated, evaporated, the residue was dissolved in MTBE (200 mL), dried over Na₂SO₄, filtered and evaporated in vacuo to give crude product, which was purified by gradient chromatography (hexane-MTBE) to obtain tert-butyl rac-(3S)-6-(2,3-dihydrobenzofuran-5-yl)-3-methyl-3,4-dihydro-2H-pyridine-1-carboxylate (2.3 g, 7.29 mmol, 59.78% yield).

LCMS(ESI): [M-Boc]⁺ m/z: calcd 216.2; found 216.2; Rt=1.659 min.

### Step 2: The Synthesis of rac-(3S)-6-(2,3-Dihydrobenzofuran-5-yl)-3-methyl-2,3,4,5-tetrahydropyridine

tert-butyl rac-(3S)-6-(2,3-dihydrobenzofuran-5-yl)-3-methyl-3,4-dihydro-2H-pyridine-1-carboxylate (2.3 g, 7.29 mmol) was taken up with Trifluoroacetic acid (20 g, 175.40 mmol, 13.42 mL). The reaction mixture was stirred at 25° C. for 1 hr, evaporated and diluted with brine (5 mL). pH of the solution was adjusted to 8-9 with NaHCO₃ and extracted with DCM (3*15 mL), dried over Na₂SO₄ and evaporated in vacuo to give rac-(3S)-6-(2,3-dihydrobenzofuran-5-yl)-3-methyl-2,3,4,5-tetrahydropyridine (0.8 g, 3.72 mmol, 50.96% yield).

LCMS(ESI): [M+H]⁺ m/z: calcd 216.2; found 216.2; Rt=0.651 min.

### Step 3: The Synthesis of rac-(2R,5S)-2-(2,3-Dihydrobenzofuran-5-yl)-5-methyl-piperidine

NaBH₄ (175.73 mg, 4.64 mmol, 163.62 μL) was added in portions to a stirred solution of rac-(3S)-6-(2,3-dihydrobenzofuran-5-yl)-3-methyl-2,3,4,5-tetrahydropyridine (1 g, 4.64 mmol) in MeOH (30 mL) at 25° C. The resulting mixture was stirred at 25° C. for 1 hr, and then evaporated in vacuo. The residue was diluted with brine (5 mL) and extracted with dichloromethane (2*20 mL). The combined organic extracts were dried over sodium sulphate and evaporated in vacuo to afford rac-(2R,5S)-2-(2,3-dihydrobenzofuran-5-yl)-5-methyl-piperidine (1 g, 4.60 mmol, 99.07% yield).

LCMS(ESI): [M+H]⁺ m/z: calcd 218.2; found 218.2; Rt=0.678 min.

### Step 4: The Synthesis of 2,2,2-Trifluoroethyl 2-oxo-2-[rac-(2R,5S)-2-(2,3-dihydrobenzofuran-5-yl)-5-methyl-1-piperidyl]acetate

To a solution of rac-(2R,5S)-2-(2,3-dihydrobenzofuran-5-yl)-5-methyl-piperidine (1 g, 4.60 mmol) and TEA (2.33 g, 23.01 mmol, 3.21 mL) in THF (40 mL), 2,2,2-trifluoroethyl 2-chloro-2-oxo-acetate (2.19 g, 11.50 mmol) was added portionwise at 0° C. The resulting mixture was stirred for 3 hr. LCMS showed full conversion of SM. The reaction mixture was directly used for the next step.

LCMS(ESI): [M+H]⁺ m/z: calcd 372.2; found 372.4; Rt=1.329 min.

### Step 5: The Synthesis of 2-Oxo-2-[rac-(2R,5S)-2-(2,3-dihydrobenzofuran-5-yl)-5-methyl-1-piperidyl]acetamide

Through a solution of 2,2,2-trifluoroethyl 2-oxo-2-[rac-(2R,5S)-2-(2,3-dihydrobenzofuran-5-yl)-5-methyl-1-piperidyl]acetate (1.7 g, 4.58 mmol) in THF (50 mL), ammonia was bubbled during 10 min at 0° C. The formed precipitate was filtered off, washed with THF (30 mL) and the solvent was evaporated in vacuo to give crude product (1.5 g), which was purified by gradient chromatography (CHCl₃-ACN) to afford 2-oxo-2-[rac-(2R,5S)-2-(2,3-dihydrobenzofuran-5-yl)-5-methyl-1-piperidyl]acetamide (0.85 g, 2.95 mmol, 64.39% yield).

LCMS(ESI): [M+H]⁺ m/z: calcd 289.2; found 289.2; Rt=0.934 min.

### Step 6: The Synthesis of N-(4-Amino-2-tetrahydropyran-2-yl-pyrazolo[4,3-c]pyridin-7-yl)-2-oxo-2-[rac-(2R,5S)-2-(2,3-dihydrobenzofuran-5-yl)-5-methyl-1-piperidyl]acetamide

To a mixture of 2-oxo-2-[rac-(2R,5S)-2-(2,3-dihydrobenzofuran-5-yl)-5-methyl-1-piperidyl]acetamide (0.15 g, 520.22 μmol), 7-bromo-2-tetrahydropyran-2-yl-pyrazolo[4,3-c]pyridin-4-amine (200.96 mg, 676.28 μmol), Copper (6.61 mg, 104.04 μmol), Copper (I) iodide (59.45 mg, 312.13 μmol, 10.58 μL), Cesium carbonate (203.40 mg, 624.26 μmol) and rac-(1R,2R)—N1,N2-dimethylcyclohexane-1,2-diamine (44.40 mg, 312.13 μmol), Dioxane (5 mL) was added. The resulting mixture was evacuated, refiled with Argon three time, heated at 100° C. for 18 hr and cooled. The inorganic precipitate was filtered off and washed with DCM (50 mL) to give N-(4-amino-2-tetrahydropyran-2-yl-pyrazolo[4,3-c]pyridin-7-yl)-2-oxo-2-[rac-(2R,5S)-2-(2,3-dihydrobenzofuran-5-yl)-5-methyl-1-piperidyl]acetamide (0.25 g, crude).

This substance was used for the next step without further purification.

LCMS(ESI): [M+H]⁺ m/z: calcd 505.2; found 505.2; Rt=1.160 min.

### Step 7: The Synthesis of N-(4-Amino-1H-pyrazolo[4,3-c]pyridin-7-yl)-2-oxo-2-[rac-(2R,5S)-2-(2,3-dihydrobenzofuran-5-yl)-5-methyl-1-piperidyl]acetamide (Compound 206)

To a solution of N-(4-amino-2-tetrahydropyran-2-yl-pyrazolo[4,3-c]pyridin-7-yl)-2-oxo-2-[rac-(2R,5S)-2-(2,3-dihydrobenzofuran-5-yl)-5-methyl-1-piperidyl]acetamide (300 mg, 594.55 μmol) in DCM (20 mL) at 0° C., SnCl₄ (1.55 g, 5.95 mmol, 695.84 μL) was added portionwise. The reaction mixture was stirred for 2 hr taken up with water 20 ml and ˜10 mL of aqueous solution of NH₃. The product was extracted with DCM, dried over Na₂SO₄ and evaporated in vacuo to give crude product (200 mg), which was purified by HPLC (Device (Mobile Phase, Column): SYSTEM 30-30-65% 0-1-5 min H₂O/MeOH/0.1% NH₄OH, flow: 30 mL/min (loading pump 4 mL/min methanol); column: XBridge BEH C18 100×19 mm, 5 um) and re-purified (Device (Mobile Phase, Column): SYSTEM 10-10-5% 0-1-5 min H₂O/ACN/0.1% NH₄OH, flow: 30 mL/min (loading pump 4 mL/min acetonitrile); column: XBridge BEH C18 100×19 mm, 5 um) to obtain N-(4-amino-1H-pyrazolo[4,3-c]pyridin-7-yl)-2-oxo-2-[rac-(2R,5S)-2-(2,3-dihydrobenzofuran-5-yl)-5-methyl-1-piperidyl]acetamide (24 mg, 57.08 μmol, 9.60% yield).

¹H NMR (600 MHz, DMSO-d₆) δ (ppm) 0.68-1.06 (m, 3H), 1.20-1.35 (m, 1H), 1.59-2.24 (m, 4H), 2.65-3.26 (m, 3H), 3.37-4.09 (m, 1H), 4.38-4.57 (m, 2H), 4.87-5.85 (m, 1H), 6.33-6.82 (m, 3H), 6.84-7.29 (m, 2H), 7.46-7.76 (m, 1H), 8.08-8.25 (m, 1H), 9.52-10.58 (m, 1H), 12.56-13.37 (m, 1H).

LCMS(ESI): [M+H]⁺ m/z: calcd 421.2; found 421.2; Rt=2.633 min.

### Example 184. N-(4-Amino-1H-pyrazolo[4,3-c]pyridin-7-yl)-2-oxo-2-[rac-(2R,5S)-5-methyl-2-(p-tolyl)-1-piperidyl]acetamide (Compound 207)

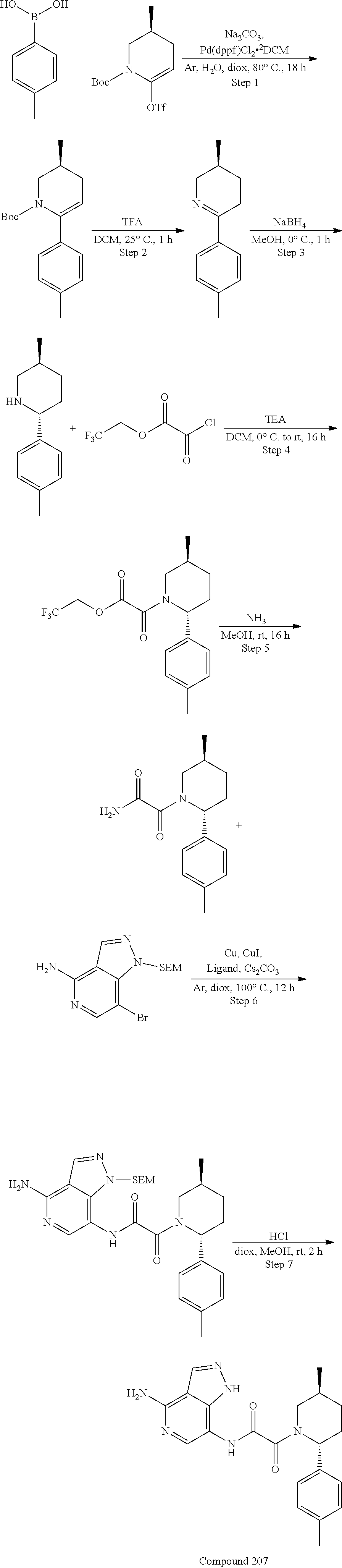

### Step 1: The Synthesis of tert-Butyl rac-(3S)-3-methyl-6-(p-tolyl)-3,4-dihydro-2H-pyridine-1-carboxylate

tert-Butyl (3S)-3-methyl-6-(trifluoromethylsulfonyloxy)-3,4-dihydro-2H-pyridine-1-5 carboxylate (9.14 g, 26.48 mmol), Sodium carbonate (7.02 g, 66.20 mmol, 2.77 mL) and water (39.56 mL) were added to a solution of p-tolylboronic acid (3 g, 22.07 mmol) in 1,4-dioxane (158.23 mL). The resulting mixture was evacuated and then backfilled with argon. This operation was repeated two times, then Pd(dppf)Cl2·DCM (900.99 mg, 1.10 mmol) was added and the reaction mixture was stirred under argon at 80° C. for 18 hr, then cooled down and concentrated in vacuo. The residue was diluted with MTBE (60 mL) and stirred for 0.5 hr. After the main part of the residue dissolved, anhydrous sodium sulfate was added, and the resulting mixture was filtered. The filtercake was additionally washed with MTBE (3*20 mL) and discarded. The filtrate was concentrated in vacuo to afford crude tert-butyl rac-(3S)-3-methyl-6-(p-tolyl)-3,4-dihydro-2H-pyridine-1-carboxylate (4.5 g, 15.66 mmol, 70.96% yield) as brown gum, which was used directly in the next step.

LCMS(ESI): [M−tBu]⁺ m/z: calcd 232.2; found 232.2; Rt=1.776 min.

### Step 2: The Synthesis of rac-(3S)-3-Methyl-6-(p-tolyl)-2,3,4,5-tetrahydropyridine

Trifluoroacetic acid (11.90 g, 104.39 mmol, 8.04 mL) was added in one portion to a stirred solution of tert-butyl rac-(3S)-3-methyl-6-(p-tolyl)-3,4-dihydro-2H-pyridine-1-carboxylate (2 g, 6.96 mmol) in dichloromethane. The resulting solution was stirred at 25° C. for 0.5 hr, and then concentrated in vacuo. The residue was diluted with water (30 mL). The resulting solution of TFA salt of the product was decanted from dark-brown oily residue, which was additionally rinsed with water (2*10 mL). The combined aqueous solution was basified to pH 11-12 with 10% aqueous sodium carbonate solution and extracted with dichloromethane (2*20 mL). The combined organic extracts were dried over sodium sulfate and concentrated in vacuo to afford rac-(3S)-3-methyl-6-(p-tolyl)-2,3,4,5-tetrahydropyridine (1.13 g, 6.03 mmol, 86.70% yield) as brown gum, which was directly used in the next step.

LCMS(ESI): [M+H]⁺ m/z: calcd 188.2; found 188.2; Rt=0.818 min.

### Step 3: The Synthesis of rac-(2R,5S)-5-Methyl-2-(p-tolyl)piperidine

Sodium Borohydride (1.14 g, 30.17 mmol, 1.06 mL) was added in one portion at 0° C. to a stirred solution of rac-(3S)-3-methyl-6-(p-tolyl)-2,3,4,5-tetrahydropyridine (1.13 g, 6.03 mmol) in methanol (14.37 mL). The reaction mixture was stirred at 0° C. for 1 hr, and then concentrated in vacuo. The residue was diluted with water (10 mL) and extracted with dichloromethane (2*15 mL). The combined organic extracts were dried over sodium sulfate and concentrated in vacuo to afford crude rac-(2R,5S)-5-methyl-2-(p-tolyl)piperidine (1.05 g, 5.55 mmol, 91.93% yield).

LCMS(ESI): [M+H]⁺ m/z: calcd 190.2; found 190.2; Rt=0.903 min.

### Step 4: The Synthesis of Ethyl 2-oxo-2-[rac-(2R,5S)-5-methyl-2-(p-tolyl)-1-piperidyl]acetate

rac-(2R,5S)-5-Methyl-2-(p-tolyl)piperidine (1.05 g, 5.55 mmol) and TEA (561.29 mg, 5.55 mmol, 773.12 μL) was dissolved in DCM, cooled with ice-water bath and then ethyl 2-chloro-2-oxo-acetate (757.33 mg, 5.55 mmol, 619.75 μL) in 20 mL of DCM was added dropwise in 10 min. Solution was stirred overnight. Reaction mixture was washed with aqueous solution of NaHCO₃, dried over sodium sulphate and evaporated to give ethyl 2-oxo-2-[rac-(2R,5S)-5-methyl-2-(p-tolyl)-1-piperidyl]acetate (1.2 g, 4.15 mmol, 74.76% yield).

LCMS(ESI): [M+H]⁺ m/z: calcd 290.2; found 290.2; Rt=1.424 min.

### Step 5: The Synthesis of 2-Oxo-2-[rac-(2R,5S)-5-methyl-2-(p-tolyl)-1-piperidyl]acetamide

Ethyl 2-oxo-2-[rac-(2R,5S)-5-methyl-2-(p-tolyl)-1-piperidyl]acetate (1.2 g, 4.15 mmol) was dissolved in NH₃/methanol (30 mL) solution (10% by weight) and stirred overnight, then concentrated in vacuo to give 2-oxo-2-[rac-(2R,5S)-5-methyl-2-(p-tolyl)-1-piperidyl]acetamide (0.55 g, 2.11 mmol, 50.95% yield).

LCMS(ESI): [M+H]⁺ m/z: calcd 261.2; found 261.2; Rt=1.200 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-5-methyl-2-(p-tolyl)-1-piperidyl]acetamide

7-Bromo-1-(2-trimethylsilylethoxymethyl)pyrazolo[4,3-c]pyridin-4-amine (329.67 mg, 960.32 μmol), 2-oxo-2-[rac-(2R,5S)-5-methyl-2-(p-tolyl)-1-piperidyl]acetamide (0.25 g, 960.32 μmol), Copper (I) iodide (54.87 mg, 288.10 μmol, 9.76 μL), Cesium carbonate (625.78 mg, 1.92 mmol) and (1R,2R)—N1,N2-dimethylcyclohexane-1,2-diamine (61.47 mg, 432.14 μmol) were mixed in Dioxane (3 mL) under argon, and then stirred overnight at 100° C. for 12 hr in vial. Reaction mixture was filtered, then evaporated. Reaction mixture was filtered, then evaporated. The residue was dissolved in 15 mL of DCM washed with an aqueous ammonium solution, dried over sodium sulfate and evaporated to give N-[4-amino-1-(2-trimethylsilylethoxymethyl)pyrazolo[4,3-c]pyridin-7-yl]-2-oxo-2-[rac-(2R,5S)-5-methyl-2-(p-tolyl)-1-piperidyl]acetamide (0.47 g, 899.15 μmol, 93.63% yield).

LCMS(ESI): [M+H]⁺ m/z: calcd 523.2; found 523.2; Rt=1.285 min.

### Step 7: The Synthesis of N-(4-Amino-1H-pyrazolo[4,3-c]pyridin-7-yl)-2-oxo-2-[rac-(2R,5S)-5-methyl-2-(p-tolyl)-1-piperidyl]acetamide (Compound 207)

N-[4-Amino-1-(2-trimethylsilylethoxymethyl)pyrazolo[4,3-c]pyridin-7-yl]-2-oxo-2-[rac-(2R,5S)-5-methyl-2-(p-tolyl)-1-piperidyl]acetamide (0.47 g, 899.15 μmol) was dissolved in MeOH (1 mL) and diox/HCl (17.98 mmol, 1 mL) was added thereto. Then it was stirred at rt for 2 hr. The reaction mixture was evaporated. Crude product was purified by HPLC to afford N-(4-amino-1H-pyrazolo[4,3-c]pyridin-7-yl)-2-oxo-2-[rac-(2R,5S)-5-methyl-2-(p-tolyl)-1-piperidyl]acetamide (0.0337 g, 85.87 μmol, 9.55% yield).

### Chromatography Data:

2-10 min 30-55% water-methanol+NH₃; flow: 30 mL/min ((loading pump 4 mL methanol)

¹H NMR (600 MHz, dmso) δ 0.67-1.05 (m, 3H), 1.19-1.37 (m, 1H), 1.61-1.76 (m, 1H), 1.77-1.95 (m, 1H), 1.98-2.32 (m, 5H), 2.70-3.25 (m, 1H), 3.41-4.12 (m, 1H), 4.89-5.68 (m, 1H), 6.15-6.80 (m, 2H), 6.85-7.13 (m, 1H), 7.15-7.27 (m, 3H), 7.49-7.76 (m, 1H), 8.10-8.27 (m, 1H), 9.18-11.08 (m, 1H), 11.97-13.71 (m, 1H).

LCMS(ESI): [M+H]⁺ m/z: calcd 393.2; found 393.2; Rt=1.328 min.

### Example 185. N-(4-Amino-1H-pyrazolo[4,3-c]pyridin-7-yl)-2-oxo-2-[rac-(2R,5S)-2-(3,4-dimethylphenyl)-5-methyl-1-piperidyl]acetamide (Compound 208)

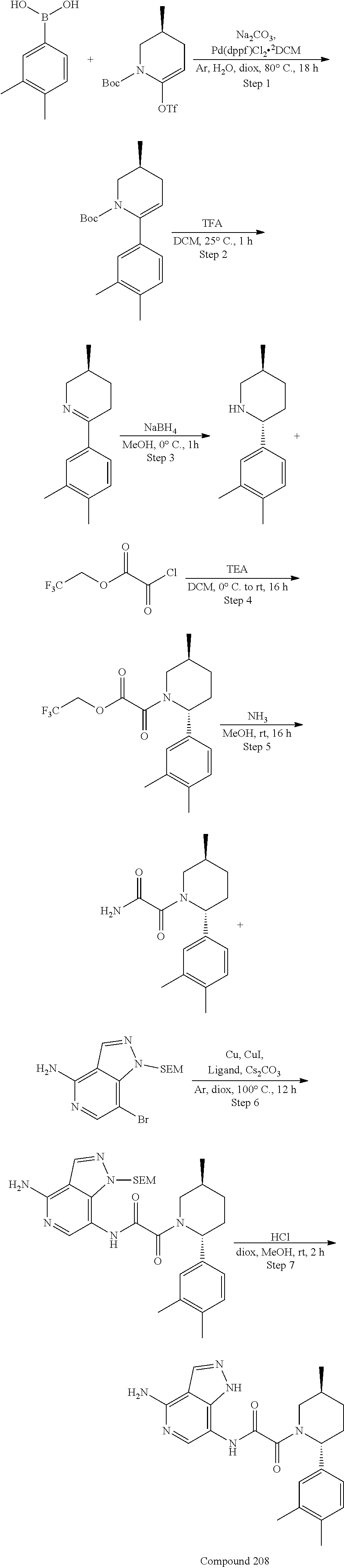

### Step 1: The Synthesis of tert-Butyl rac-(3S)-6-(3,4-dimethylphenyl)-3-methyl-3,4-dihydro-2H-pyridine-1-carboxylate

tert-Butyl (3S)-3-methyl-6-(trifluoromethylsulfonyloxy)-3,4-dihydro-2H-pyridine-1-carboxylate (5.53 g, 16.00 mmol), Sodium carbonate (4.24 g, 40.00 mmol, 1.67 mL) and water (39.78 mL) were added to a solution of (3,4-dimethylphenyl) boronic acid (2 g, 13.33 mmol) in 1,4-dioxane (159.11 mL). The resulting mixture was evacuated and then backfilled with argon. This operation was repeated two times, then Pd(dppf)Cl2 DCM (544.49 mg, 666.75 μmol) was added and the reaction mixture was stirred under argon at 80° C. for 18 hr, then cooled down and concentrated in vacuo. The residue was diluted with MTBE (60 mL) and stirred for 0.5 hr. After the main part of the residue had dissolved, anhydrous sodium sulfate was added, and the resulting mixture was filtered. The filtercake was additionally washed with MTBE (3*20 mL) and discarded. The filtrate was concentrated in vacuo to afford crude tert-butyl rac-(3S)-6-(3,4-dimethylphenyl)-3-methyl-3,4-dihydro-2H-pyridine-1-carboxylate (3.8 g, 12.61 mmol, 94.54% yield) as brown gum, which was used directly in the next step.

LCMS(ESI): [M−tBu]⁺ m/z: calcd 246.2; found 246.2; Rt=1.667 min.

### Step 2: The Synthesis of rac-(3S)-6-(3,4-Dimethylphenyl)-3-methyl-2,3,4,5-tetrahydropyridine

Trifluoroacetic acid (11.35 g, 99.53 mmol, 7.67 mL) was added in one portion to a stirred solution of tert-butyl rac-(3S)-6-(3,4-dimethylphenyl)-3-methyl-3,4-dihydro-2H-pyridine-1-carboxylate (2 g, 6.64 mmol) in dichloromethane. The resulting solution was stirred at 25° C. for 0.5 hr, and then concentrated in vacuo. The residue was diluted with water (30 mL). The resulting solution of TFA salt of the product was decanted from dark-brown oily residue, which was additionally rinsed with water (2*10 mL). The combined aqueous solution was basified to pH 11-12 with 10% aqueous sodium carbonate solution and extracted with dichloromethane (2*20 mL). The combined organic extracts were dried over sodium sulfate and concentrated in vacuo to afford rac-(3S)-6-(3,4-dimethylphenyl)-3-methyl-2,3,4,5-tetrahydropyridine (1.2 g, 5.96 mmol, 89.84% yield) as brown gum, which was directly used in the next step.

LCMS(ESI): [M+H]⁺ m/z: calcd 202.2; found 202.2; Rt=0.896 min.

### Step 3: The Synthesis of rac-(2R,5S)-2-(3,4-Dimethylphenyl)-5-methyl-piperidine

Sodium Borohydride (1.13 g, 29.81 mmol, 1.05 mL) was added in one portion at 0° C. to a stirred solution of rac-(3S)-6-(3,4-dimethylphenyl)-3-methyl-2,3,4,5-tetrahydropyridine (1.2 g, 5.96 mmol) in methanol (14.38 mL). The reaction mixture was stirred at 0° C. for 1 hr, and then concentrated in vacuo. The residue was diluted with water (10 mL) and extracted with dichloromethane (2*15 mL). The combined organic extracts were dried over sodium sulfate and concentrated in vacuo to afford crude rac-(2R,5S)-2-(3,4-dimethylphenyl)-5-methyl-piperidine (1.1 g, 5.41 mmol, 90.76% yield).

LCMS(ESI): [M+H]⁺ m/z: calcd 204.2; found 204.2; Rt=0.975 min.

### Step 4: The Synthesis of Ethyl 2-oxo-2-[rac-(2R,5S)-2-(3,4-dimethylphenyl)-5-methyl-1-piperidyl]acetate

rac-(2R,5S)-2-(3,4-Dimethylphenyl)-5-methyl-piperidine (1.1 g, 5.41 mmol) and TEA (547.45 mg, 5.41 mmol, 754.06 μL) was dissolved in DCM (30 mL), cooled with ice-water bath and then ethyl 2-chloro-2-oxo-acetate (738.66 mg, 5.41 mmol, 604.47 μL) in 20 mL of DCM was added dropwise in 10 min. Solution was stirred overnight. Reaction mixture was washed with aqueous solution of NaHCO₃, dried over sodium sulphate and evaporated to give ethyl 2-oxo-2-[rac-(2R,5S)-2-(3,4-dimethylphenyl)-5-methyl-1-piperidyl]acetate (1.5 g, 4.94 mmol, 91.39% yield).

LCMS(ESI): [M+H]⁺ m/z: calcd 304.2; found 304.2; Rt=1.474 min.

### Step 5: The Synthesis of 2-Oxo-2-[rac-(2R,5S)-2-(3,4-dimethylphenyl)-5-methyl-1-piperidyl]acetamide

Ethyl 2-oxo-2-[rac-(2R,5S)-2-(3,4-dimethylphenyl)-5-methyl-1-piperidyl]acetate (1.26 g, 4.15 mmol) was dissolved in NH₃/methanol (30 mL) solution (10% by weight) and stirred overnight, then concentrated in vacuo to give 2-oxo-2-[rac-(2R,5S)-2-(3,4-dimethylphenyl)-5-methyl-1-piperidyl]acetamide (0.82 g, 2.99 mmol, 72.07% yield).

LCMS(ESI): [M+H]⁺ m/z: calcd 275.2; found 275.2; Rt=1.261 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-2-(3,4-dimethylphenyl)-5-methyl-1-piperidyl]acetamide

7-Bromo-1-(2-trimethylsilylethoxymethyl)pyrazolo[4,3-c]pyridin-4-amine (312.82 mg, 911.22 μmol), 2-oxo-2-[rac-(2R,5S)-2-(3,4-dimethylphenyl)-5-methyl-1-piperidyl]acetamide (0.25 g, 911.22 μmol), Copper (I) iodide (52.06 mg, 273.37 μmol, 9.26 μL), Cesium carbonate (593.79 mg, 1.82 mmol) and (1R,2R)—N1,N2-dimethylcyclohexane-1,2-diamine (58.33 mg, 410.05 μmol) were mixed in Dioxane (3 mL) under argon, and then stirred overnight at 100° C. for 12 hr in vial. Reaction mixture was filtered, then evaporated. Reaction mixture was filtered, then evaporated. The residue was dissolved in 15 mL of DCM washed with an aqueous ammonium solution, dried over sodium sulfate and evaporated to give N-[4-amino-1-(2-trimethylsilylethoxymethyl)pyrazolo[4,3-c]pyridin-7-yl]-2-oxo-2-[rac-(2R,5S)-2-(3,4-dimethylphenyl)-5-methyl-1-piperidyl]acetamide (0.43 g, 801.13 μmol, 87.92% yield.

LCMS(ESI): [M+H]⁺ m/z: calcd 537.2; found 537.4; Rt=1.316 min.

### Step 7: The Synthesis of N-(4-Amino-1H-pyrazolo[4,3-c]pyridin-7-yl)-2-oxo-2-[rac-(2R,5S)-2-(3,4-dimethylphenyl)-5-methyl-1-piperidyl]acetamide (Compound 208)

N-[4-Amino-1-(2-trimethylsilylethoxymethyl)pyrazolo[4,3-c]pyridin-7-yl]-2-oxo-2-[rac-(2R,5S)-2-(3,4-dimethylphenyl)-5-methyl-1-piperidyl]acetamide (0.43 g, 801.13 μmol) was dissolved in MeOH (1 mL) and diox/HCl (16.02 mmol, 1 mL) was added thereto. Then it was stirred at rt for 2 hr. The reaction mixture was evaporated. Crude product was purified by HPLC to afford N-(4-amino-1H-pyrazolo[4,3-c]pyridin-7-yl)-2-oxo-2-[rac-(2R,5S)-2-(3,4-dimethylphenyl)-5-methyl-1-piperidyl]acetamide (0.026 g, 63.96 μmol, 7.98% yield)

### Chromatography Data:

2-10 min 30-55% water-methanol+NH₃; 30 mL/min ((loading pump 4 mL methanol)

¹H NMR (600 MHz, dmso) δ 0.71-1.07 (m, 3H), 1.21-1.37 (m, 1H), 1.43-2.05 (m, 3H), 2.14-2.25 (m, 6H), 2.73-3.24 (m, 1H), 3.35-4.10 (m, 2H), 4.61-5.68 (m, 1H), 6.65-7.55 (m, 5H), 7.68-8.38 (m, 2H), 9.56-10.61 (m, 1H), 12.59-13.42 (m, 1H).

LCMS(ESI): [M+H]⁺ m/z: calcd 407.2; found 407.2; Rt=1.427 min.

### Example 186. N-(4-Amino-1H-pyrazolo[4,3-c]pyridin-7-yl)-2-oxo-2-[rac-(2R,4S)-4-methyl-2-phenyl-1-piperidyl]acetamide (Compound 209)

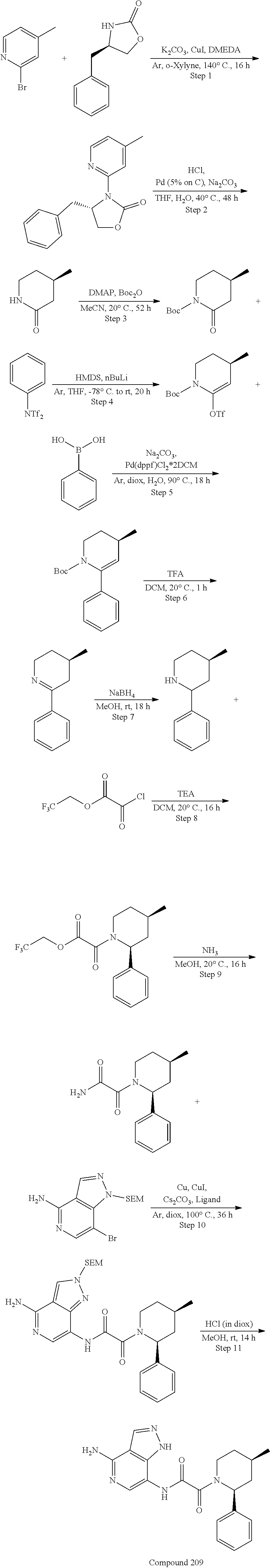

### Step 1: The Synthesis of rac-(4R)-4-Benzyl-3-(4-methyl-2-pyridyl) oxazolidin-2-one

2-Bromo-4-methyl-pyridine (9.7 g, 56.39 mmol, 6.28 mL), rac-(4R)-4-benzyloxazolidin-2-one (9.99 g, 56.39 mmol), Copper (I) iodide (536.96 mg, 2.82 mmol, 95.54 μL), N,N-Dimethylethylenediamine (497.06 mg, 5.64 mmol, 615.94 μL) and Potassium carbonate-granular (15.59 g, 112.78 mmol, 6.81 mL) were mixed in o-xylene (120 mL) and the reaction mixture was purged with argon. The reaction mixture was heated at 140° C. overnight. The reaction mixture was cooled, filtered and concentrated in vacuo. The residue (18 g) was purified by FCC (Interchim; 120 g SiO₂, CHCl₃—CH₃CN from 0˜100%, flow rate=70 mL/min, cv=4.2) to obtain rac-(4R)-4-benzyl-3-(4-methyl-2-pyridyl) oxazolidin-2-one (8.45 g, 31.49 mmol, 55.85% yield).

¹H NMR (500 MHz, DMSO-d₆) δ (ppm) 2.35 (s, 3H), 2.99 (m, 1H), 3.12 (m, 1H), 4.21 (m, 1H), 4.39 (m, 1H), 5.5 (m, 1H), 7.01 (d, 1H), 7.23 (m, 5H), 7.89 (s, 1H), 8.31 (d, 1H).

### Step 2: The Synthesis of rac-(4S)-4-Methylpiperidin-2-one

To a 500 mL autoclave rac-(4R)-4-benzyl-3-(4-methyl-2-pyridyl) oxazolidin-2-one (8.45 g, 31.49 mmol) and Palladium, 5% on activated carbon paste, 5R437 (3.35 g, 3.15 mmol, 10% purity) were charged. THF (100 mL) and Water (100 mL) were added thereto followed by addition of Hydrochloric acid ACS grade 36-38% (7.66 g, 75.58 mmol, 9.57 mL, 36% purity). The autoclave was pressurized and depressurized three times with hydrogen gas before the final hydrogen pressure was set to 50 atm. The reaction mixture was stirred at 50 atm at 40° C. for 48 hr. The autoclave was cooled to room temperature and carefully depressurized. The reaction mixture was carefully poured onto Sodium carbonate (13.35 g, 125.97 mmol, 5.27 mL) and the resulting mixture was filtered. The filter cake was rinsed with THF (150 mL) and the filtrate was concentrated in vacuo. The residue was re-dissolved in water (75 mL) and extracted with DCM (2*100 mL). Combined organic layers were dried over Na₂SO₄, filtered, and concentrated in vacuo. The residue was purified by FCC (Interchim; 120 g SiO₂, MTBE-MeOH from 0˜100%, flow rate=70 mL/min, cv=10.3) to obtain rac-(4S)-4-methylpiperidin-2-one (3 g, 26.51 mmol, 84.18% yield).

¹H NMR (500 MHz, CDCl₃) δ (ppm) 1.01 (s, 3H), 1.22 (m, 1H), 1.87 (m, 3H), 2.38 (m, 1H), 3.31 (m, 2H), 6.16 (br s, 1H).

### Step 3: The Synthesis of tert-Butyl rac-(4S)-4-methyl-2-oxo-piperidine-1-carboxylate

rac-(4S)-4-Methylpiperidin-2-one (3 g, 26.51 mmol) was dissolved in MeCN (29.00 mL) and DMAP (323.89 mg, 2.65 mmol) was added thereto. Di-tert-butyl dicarbonate (7.52 g, 34.47 mmol, 7.91 mL) was added dropwise and the resulting mixture was stirred at 20° C. for 52 hr. The reaction mixture was concentrated in vacuo and the residue was dissolved in DCM (70 mL). The resulting solution was washed with 10% aq.citric acid solution (20 mL), dried over Na₂SO₄, filtered and concentrated in vacuo to obtain tert-butyl rac-(4S)-4-methyl-2-oxo-piperidine-1-carboxylate (5.3 g, crude).

LCMS(ESI): [M−tBu]⁺ m/z: calcd 158.0; found 158.0; Rt=0.921 min.

### Step 4: The Synthesis of tert-Butyl rac-(4S)-4-methyl-6-(trifluoromethylsulfonyloxy)-3,4-dihydro-2H-pyridine-1-carboxylate

Hexamethyldisilazane (1.85 g, 11.44 mmol, 2.39 mL) was dissolved in THF (16 mL) and the resulting mixture was cooled to −78° C. under an argon atmosphere. n-butyllithium (720.88 mg, 11.25 mmol, 4.51 mL) was added dropwise at −78° C. and the resulting solution was stirred for 30 min. A solution of tert-butyl rac-(4S)-4-methyl-2-oxo-piperidine-1-carboxylate (2 g, 9.38 mmol) in THF (5 mL) was added dropwise at −78° C. and the resulting mixture was stirred at −78° C. for 1.5 hr. 1,1,1-trifluoro-N-phenyl-N-(trifluoromethylsulfonyl) methanesulfonamide (3.52 g, 9.85 mmol) was added in one portion to the previous mixture at −78° C. and the resulting mixture was stirred at −78° C. for 30 min. The resulting mixture was allowed to warm to room temperature and stirred overnight. The reaction mixture was diluted with water (35 mL) and MTBE (65 mL) and the organic layer was separated. The aqueous layer was additionally extracted with MTBE (65 mL) and combined organic layers were washed with 10% aqueous sodium hydroxide solution (3*25 mL), dried over Na₂SO₄ and concentrated in vacuo. The residue was diluted with hexane and stirred for 30 min. The resulting cloudy solution was decanted from the oily residue, filtered through a short pad of silica gel (20 g of dry silica gel), and evaporated to obtain crude tert-butyl rac-(4S)-4-methyl-6-(trifluoromethylsulfonyloxy)-3,4-dihydro-2H-pyridine-1-carboxylate (1.6 g, 4.63 mmol, 49.41% yield) which was used further without purification.

LCMS(ESI): [M−tBu]⁺ m/z: calcd 290.0; found 290.0; Rt=1.475 min.

### Step 5: The Synthesis of tert-Butyl rac-(4S)-4-methyl-6-phenyl-3,4-dihydro-2H-pyridine-1-carboxylate

tert-Butyl rac-(4S)-4-methyl-6-(trifluoromethylsulfonyloxy)-3,4-dihydro-2H-pyridine-1-carboxylate (1.6 g, 4.63 mmol), phenylboronic acid (706.15 mg, 5.79 mmol) and Sodium carbonate (1.23 g, 11.58 mmol, 484.87 μL) were mixed together in a mixture of Dioxane (18 mL) and Water (6 mL). The resulting mixture was evacuated and backfilled three times with argon. Pd(dppf)Cl₂·DCM (189.18 mg, 231.66 μmol) was added to the previous mixture and the resulting mixture was heated at 90° C. overnight. The reaction mixture was concentrated in vacuo and the residue was diluted with water (50 mL). The resulting mixture was extracted with EtOAc (2*75 mL) and combined organic layers were washed with brine (50 mL), dried over Na₂SO₄, filtered and concentrated in vacuo.

LCMS(ESI): [M−tBu]⁺ m/z: calcd 218.2; found 218.2; Rt=1.549 min.

### Step 6: The Synthesis of rac-(4S)-4-Methyl-6-phenyl-2,3,4,5-tetrahydropyridine

Trifluoroacetic acid (7.40 g, 64.90 mmol, 5 mL) was added in one portion to a stirred solution of tert-butyl rac-(4S)-4-methyl-6-phenyl-3,4-dihydro-2H-pyridine-1-carboxylate (2 g, 7.32 mmol) in dichloromethane (10 mL). The resulting solution was stirred at 20° C. for 1 hr, and then concentrated in vacuo. The residue was diluted with water (50 mL). The resulting solution of TFA salt of the product was decanted from dark-brown oily residue, which was additionally rinsed with water (2*20 mL). The combined aqueous solution was filtered through a cotton pad to remove traces of oily impurities, then basified to pH 11-12 with 10% aqueous sodium carbonate solution and extracted with dichloromethane (2*40 mL). The combined organic extracts were dried over sodium sulfate and concentrated in vacuo to afford rac-(4S)-4-methyl-6-phenyl-2,3,4,5-tetrahydropyridine (528 mg, crude) which was directly used in the next step.

### Step 7: The Synthesis of rac-(2R,4S)-4-Methyl-2-phenyl-piperidine

rac-(4S)-4-Methyl-6-phenyl-2,3,4,5-tetrahydropyridine (528 mg, 3.05 mmol) was dissolved in MeOH (10 mL) and Sodium Borohydride (345.87 mg, 9.14 mmol, 322.04 μL) was added portionwise. The resulting mixture was stirred overnight. Water (2 mL) was added to the reaction mixture and the resulting mixture was concentrated in vacuo. Water (10 mL) was added to the residue and the resulting mixture was extracted with DCM (2*35 mL). Combined organic layers were dried over Na₂SO₄, filtered and concentrated in vacuo to obtain rac-(2R,4S)-4-methyl-2-phenyl-piperidine (446 mg, crude).

LCMS(ESI): [M+H]⁺ m/z: calcd 176.2; found 176.2; Rt=0.870 min.

### Step 8: The Synthesis of 2,2,2-Trifluoroethyl 2-oxo-2-[rac-(2R,4S)-4-methyl-2-phenyl-1-piperidyl]acetate

2,2,2-Trifluoroethyl 2-chloro-2-oxo-acetate (452.16 mg, 2.37 mmol) was added dropwise to a solution of rac-(2R,4S)-4-methyl-2-phenyl-piperidine (416 mg, 2.37 mmol) and triethylamine (288.21 mg, 2.85 mmol, 396.98 μL) in DCM (10 mL) at 0° C. After addition was complete, cooling bath was removed and resulting mixture was allowed to warm up to 20° C. and stirred for 16 hr. Then, it was washed with water, dried over Na₂SO₄ and concentrated under reduced pressure, affording 2,2,2-trifluoroethyl 2-oxo-2-[rac-(2R,4S)-4-methyl-2-phenyl-1-piperidyl]acetate (0.7 g, crude) which was used in the next step without further purification.

LCMS(ESI): [M+H]⁺ m/z: calcd 330.2; found 330.2; Rt=1.529 min.

### Step 9: The Synthesis of 2-Oxo-2-[rac-(2R,4S)-4-methyl-2-phenyl-1-piperidyl]acetamide

A solution of 2,2,2-trifluoroethyl 2-oxo-2-[rac-(2R,4S)-4-methyl-2-phenyl-1-piperidyl]acetate (0.7 g, 2.13 mmol) in Methanol/NH₃ (5N) (10 mL) was stirred at 20° C. for 16 hr. The solvent was evaporated and the residue was purified by HPLC (2-10 min 30-90% H₂O-MeOH+NH₃, 30 mL/min ((loading pump 4 mL MeOH+NH₃); column: XBridge BEH C18 100*20 5 microM) to give 2-oxo-2-[rac-(2R,4S)-4-methyl-2-phenyl-1-piperidyl]acetamide (247 mg, 1.00 mmol, 47.18% yield).

LCMS(ESI): [M+H]⁺ m/z: calcd 247.2; found 247.2; Rt=0.902 min.

### Step 10: The Synthesis of N-[4-Amino-1-(2-trimethylsilylethoxymethyl)pyrazolo[4,3-c]pyridin-7-yl]-2-oxo-2-[rac-(2R,4S)-4-methyl-2-phenyl-1-piperidyl]acetamide

2-Oxo-2-[rac-(2R,4S)-4-methyl-2-phenyl-1-piperidyl]acetamide (170 mg, 690.20 μmol), 7-bromo-1-(2-trimethylsilylethoxymethyl)pyrazolo[4,3-c]pyridin-4-amine (284.33 mg, 828.24 μmol), Copper (I) iodide (26.29 mg, 138.04 μmol, 4.68 μL), Cesium carbonate (449.76 mg, 1.38 mmol) and (1R,2R)—N1,N2-dimethylcyclohexane-1,2-diamine (117.81 mg, 828.24 μmol) were mixed in dioxane (6.00 mL) under argon, and then stirred overnight at 100° C. for 36 hr in vial. The reaction mixture was filtered and the filtrate was concentrated in vacuo and the residue was purified by HPLC (2-10 min 40-80% H₂O-MeOH+FA 30 mL/min) to give N-[4-amino-1-(2-trimethylsilylethoxymethyl)pyrazolo[4,3-c]pyridin-7-yl]-2-oxo-2-[rac-(2R,4S)-4-methyl-2-phenyl-1-piperidyl]acetamide (57.9 mg, 113.82 μmol, 16.49% yield).

LCMS(ESI): [M+H]⁺ m/z: calcd 509.2; found 509.4; Rt=1.015 min.

### Step 11: The Synthesis of N-(4-Amino-1H-pyrazolo[4,3-c]pyridin-7-yl)-2-oxo-2-[rac-(2R,4S)-4-methyl-2-phenyl-1-piperidyl]acetamide (Compound 209)

To a solution of N-[4-amino-1-(2-trimethylsilylethoxymethyl)pyrazolo[4,3-c]pyridin-7-yl]-2-oxo-2-[rac-(2R,4S)-4-methyl-2-phenyl-1-piperidyl]acetamide (57.9 mg, 113.82 μmol) in MeOH (3 mL) was added Hydrogen chloride solution 4.0M in dioxane (800.00 mg, 21.94 mmol, 1 mL) at 20° C. The resulting mixture was left to stirred for 14 hr. The resulting mixture was evaporated to dryness and then submitted to reverse phase HPLC (2-10 min 20-40% H₂O-MeCN+FA, 30 mL/min ((loading pump 4 mL MeOH+FA) column: Cromatorex C18 100*19, 5 microM) to afford N-(4-amino-1H-pyrazolo[4,3-c]pyridin-7-yl)-2-oxo-2-[rac-(2R,4S)-4-methyl-2-phenyl-1-piperidyl]acetamide (17.2 mg, 40.52 μmol, 35.60% yield, HCOOH).

¹H NMR (600 MHz, DMSO-d₆) δ (ppm) 0.61-0.92 (m, 3H), 1.10-1.33 (m, 1H), 1.54-2.14 (m, 4H), 3.48-4.11 (m, 2H), 4.88-5.49 (m, 1H), 6.44-6.90 (m, 2H), 7.02-7.53 (m, 5H), 7.69-8.20 (m, 2H), 9.62-10.50 (m, 1H), 12.36-13.38 (m, 1H).

LCMS(ESI): [M+H]⁺ m/z: calcd 379.2; found 379.2; Rt=1.006 min.

### Example 187. N-(4-Amino-1H-pyrazolo[4,3-c]pyridin-7-yl)-2-oxo-2-[rac-(2R,5S)-5-methyl-2-(3-methyl-7-quinolyl)-1-piperidyl]acetamide (Compound 210)

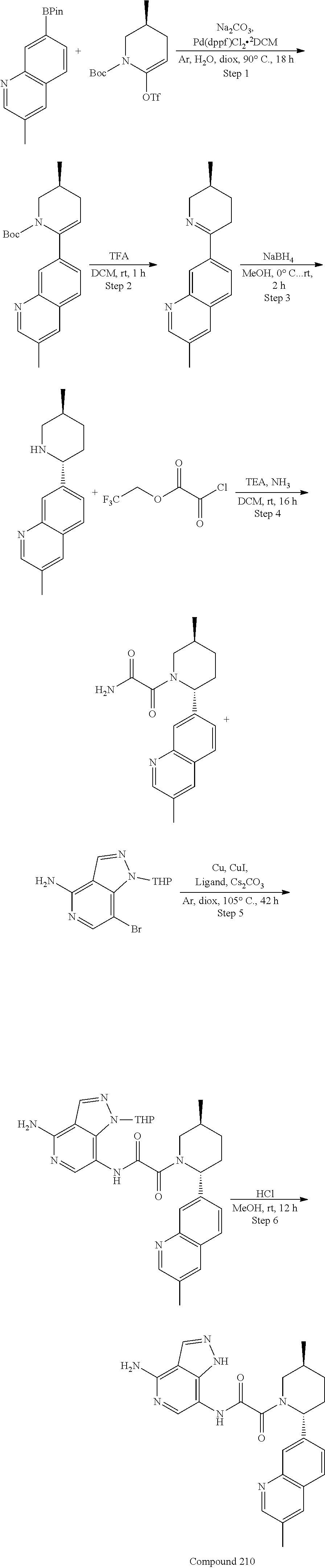

### Step 1: The Synthesis of tert-Butyl rac-(3S)-3-methyl-6-(3-methyl-7-quinolyl)-3,4-dihydro-2H-pyridine-1-carboxylate

tert-Butyl rac-(3S)-3-methyl-6-(trifluoromethylsulfonyloxy)-3,4-dihydro-2H-pyridine-1-carboxylate (8.47 g, 24.52 mmol), Sodium carbonate (7.09 g, 66.88 mmol, 2.80 mL) and water (98.94 mL) were added to a solution of crude from previous step 3-methyl-7-(4,4,5,5-tetramethyl-1,3,2-dioxaborolan-2-yl)quinoline (6 g, 22.29 mmol) in 1,4-dioxane (17.81 mL). The resulting mixture was evacuated and then backfilled with argon, this operation was repeated three times, then Pd(dppf)Cl2DCM (910.26 mg, 1.11 mmol) was added under argon. The reaction mixture was stirred under argon at 90° C. for 18 hr. The reaction mixture was cooled and then filtered. The filtercake was additionally washed with 1,4-dioxane (2*25 mL) and discarded. The combined filtrate was concentrated in vacuo to afford crude tert-butyl rac-(3S)-3-methyl-6-(3-methyl-7-quinolyl)-3,4-dihydro-2H-pyridine-1-carboxylate (8 g, crude) as black gum, which was used directly in the next step.

### Step 2: The Synthesis of 3-Methyl-7-[rac-(3S)-3-methyl-2,3,4,5-tetrahydropyridin-6-yl]quinoline

Trifluoroacetic acid (20 mL) was added to the solution of tert-Butyl rac-(3S)-3-methyl-6-(3-methyl-7-quinolyl)-3,4-dihydro-2H-pyridine-1-carboxylate (8.0 g, 23.64 mmol) in DCM (30 mL). The reaction mixture was stirred at room temperature for 1 hr. Then, the volatiles were removed under reduced pressure to afford 3-methyl-7-[rac-(3S)-3-methyl-2,3,4,5-tetrahydropyridin-6-yl]quinoline (6.0 g, crude).

### Step 3: The Synthesis of 3-Methyl-7-[rac-(2R,5S)-5-methyl-2-piperidyl]quinoline

Sodium Borohydride (285.74 mg, 7.55 mmol) was added in one portion at 0° C. to a stirred solution of 3-methyl-7-[rac-(3S)-3-methyl-2,3,4,5-tetrahydropyridin-6-yl]quinoline (1.8 g, 7.55 mmol) in methanol (30 mL). The reaction mixture was stirred at 0° C. for 1 hr, and then was allowed to warm to room temperature, and then concentrated in vacuo. The residue was diluted with water (30 mL) and extracted with dichloromethane (2*40 mL). The combined organic extracts were dried over sodium sulphate and concentrated in vacuo to afford crude 3-methyl-7-[rac-(2R,5S)-5-methyl-2-piperidyl]quinoline (1.0 g, crude).

### Step 4: The Synthesis of 2-Oxo-2-[rac-(2R,5S)-5-methyl-2-(3-methyl-7-quinolyl)-1-piperidyl]acetamide

To a solution of 3-methyl-7-[rac-(2R,5S)-5-methyl-2-piperidyl]quinoline (500.0 mg, 2.08 mmol) and Triethylamine (421.02 mg, 4.16 mmol) in DCM (20 mL), 2,2,2-trifluoroethyl 2-chloro-2-oxo-acetate (594.48 mg, 3.12 mmol) was added portionwise at 25° C. The resulting mixture was stirred for hr. LCMS showed full conversion of SM. Through resulting mixture Ammonia (35.43 mg, 2.08 mmol) was bubbled during 20 min at 25° C. The formed precipitate was filtered off, washed with DCM (30 mL) and the solvent was evaporated in vacuo to give 2-oxo-2-[rac-(2R,5S)-5-methyl-2-(3-methyl-7-quinolyl)-1-piperidyl]acetamide (500.0 mg, crude).

### Step 5: The Synthesis of N-(4-Amino-2-tetrahydropyran-2-yl-pyrazolo[4,3-c]pyridin-7-yl)-2-oxo-2-[rac-(2R,5S)-5-methyl-2-(3-methyl-7-quinolyl)-1-piperidyl]acetamide

A mixture of 2-oxo-2-[rac-(2R,5S)-5-methyl-2-(3-methyl-7-quinolyl)-1-piperidyl]acetamide (200 mg, 642.31 μmol), 7-bromo-2-tetrahydropyran-2-yl-pyrazolo[4,3-c]pyridin-4-amine (286.29 mg, 963.46 μmol), copper (5 mg, 78.68 μmol), Copper (I) iodide (130 mg, 682.59 μmol, 23.13 μL), caesium carbonate (313.91 mg, 963.46 μmol) and rac-(1R,2R)—N1,N2-dimethylcyclohexane-1,2-diamine (130 mg, 913.95 μmol) in 1,4-dioxane (6.00 mL) was stirred in a sealed vial under argon at 105° C. for 42 hr. The resulting mixture was cooled down and filtered. The filtercake was washed successively with THF (2*5 mL) and dichloromethane (3*5 mL). The combined filtrate was concentrated in vacuo to afford N-(4-amino-2-tetrahydropyran-2-yl-pyrazolo[4,3-c]pyridin-7-yl)-2-oxo-2-[rac-(2R,5S)-5-methyl-2-(3-methyl-7-quinolyl)-1-piperidyl]acetamide (650 mg, crude) as brown gum, which was used directly in the next step.

LCMS(ESI): [M+H]⁺ m/z: calcd 528.2; found 528.2; Rt=2.706 min.

### Step 6: The Synthesis of N-(4-Amino-1H-pyrazolo[4,3-c]pyridin-7-yl)-2-oxo-2-[rac-(2R,5S)-5-methyl-2-(3-methyl-7-quinolyl)-1-piperidyl]acetamide (Compound 210)

Hydrogen chloride solution 4.0M in dioxane (4.20 g, 16.01 mmol, 4 mL, 13.9% purity) was added to a stirred solution of crude from previous step N-(4-amino-2-tetrahydropyran-2-yl-pyrazolo[4,3-c]pyridin-7-yl)-2-oxo-2-[rac-(2R,5S)-5-methyl-2-(3-methyl-7-quinolyl)-1-piperidyl]acetamide (650 mg, 1.23 mmol) in methanol (4 mL) at 25° C. The resulting solution was stirred at 25° C. for 12 hr, then concentrated to dryness in vacuo and the residue was submitted to reverse phase HPLC (column: Chromatorex 18 SMB100-5T 100×19 mm 5 um; mobile phase: 5-5-30 0-2-5 min H₂O/Acetonitrile/0.2% FA; flow: 30 mL/min (loading pump 4 mL/min acetonitrile)) to afford 49 mg of crude product 90% purity by LCMS, which was re-purified by reverse phase HPLC (column: Chromatorex 18 SMB100-5T 100×19 mm 5 um; mobile phase: 40-40-90% 0-1-6 min H₂O/MeOH; flow: 30 mL/min (loading pump 4 mL/min water)) to afford Compound 210 N-(4-amino-1H-pyrazolo[4,3-c]pyridin-7-yl)-2-oxo-2-[rac-(2R,5S)-5-methyl-2-(3-methyl-7-quinolyl)-1-piperidyl]acetamide (17 mg, 34.73 μmol, 2.82% yield, HCOOH) as yellow solid.

¹H NMR (500 MHz, dmso) δ 0.94-1.10 (m, 3H), 1.28-1.55 (m, 1H), 1.65-1.82 (m, 1H), 1.87-2.03 (m, 1H), 2.10-2.31 (m, 1H), 2.35-2.42 (m, 1H), 2.43-2.46 (m, 3H), 2.73-3.25 (m, 1H), 3.72-4.22 (m, 1H), 5.12-5.95 (m, 1H), 6.58-7.06 (m, 2H), 7.45-7.61 (m, 1H), 7.61-7.78 (m, 1H), 7.81-7.95 (m, 2H), 8.06-8.26 (m, 2H), 8.64-8.79 (m, 1H), 9.58-10.70 (m, 1H), 12.62-13.45 (m, 1H).

LCMS(ESI): [M+H]⁺ m/z: calcd 444.4; found 444.4; Rt=1.551 min.

### Example 188. N-(4-Amino-1H-pyrazolo[4,3-c]pyridin-7-yl)-2-oxo-2-[rac-(2S,5S)-2-(2-ethylpyrazol-3-yl)-5-methyl-1-piperidyl]acetamide (Compound 211) and N-(4-Amino-1H-pyrazolo[4,3-c]pyridin-7-yl)-2-oxo-2-[rac-(2R,5R)-2-(2-ethylpyrazol-3-yl)-5-methyl-1-piperidyl]acetamide (Compound 285)

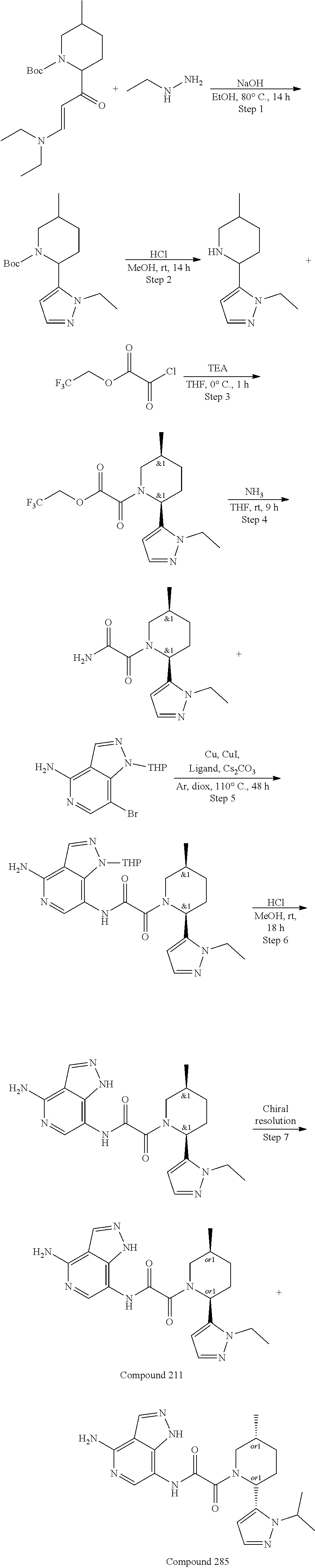

The synthesis of tert-butyl 2-(2-ethylpyrazol-3-yl)-5-methyl-piperidine-1-carboxylate is given in document: Compound 272, Compound 250.

### Step 1: The Synthesis of tert-Butyl 2-(2-ethylpyrazol-3-yl)-5-methyl-piperidine-1-carboxylate

Ethylhydrazine (2.30 g, 17.26 mmol, 2HCl) was added to a stirred solution of tert-butyl 2-[(E)-3-(diethylamino) prop-2-enoyl]-5-methyl-piperidine-1-carboxylate (3.5 g, 10.79 mmol) and Sodium hydroxide, pellets, 98% (690.33 mg, 17.26 mmol, 324.10 μL) in Ethanol (40 mL). The reaction mixture was stirred with reflux condenser at 80° C. for 14 hr, then cooled and evaporated in vacuo. The residue was combined with residue from another batch, diluted with water (100 mL) and extracted with MTBE (2*100 mL). The combined organic extracts were washed with water (2*30 mL), dried over Na₂SO₄ and concentrated in vacuo to give tert-butyl 2-(2-ethylpyrazol-3-yl)-5-methyl-piperidine-1-carboxylate (2.3 g, crude).

LCMS(ESI): [M+H]⁺ m/z: calcd 294.2; found 294.2; Rt=1.382 min.

### Step 2: The Synthesis of 2-(2-Ethylpyrazol-3-yl)-5-methyl-piperidine

Hydrogen chloride solution 4.0M in dioxane (8.00 g, 219.41 mmol, 10 mL) was added to a stirred solution of tert-butyl 2-(2-ethylpyrazol-3-yl)-5-methyl-piperidine-1-carboxylate (2.3 g, 7.84 mmol) in Methanol (15 mL). The reaction mixture was stirred at 25° C. for 14 hr, and then evaporated in vacuo. The residue was diluted with MTBE (50 mL) then filtered, washed with MTBE (50 mL) and dried in vacuo to give 2-(2-ethylpyrazol-3-yl)-5-methyl-piperidine (1.8 g, 6.76 mmol, 86.26% yield, 2HCl).

LCMS(ESI): [M+H]⁺ m/z: calcd 194.2; found 194.2; Rt=0.723 min.

### Step 3: The Synthesis of 2,2,2-Trifluoroethyl 2-oxo-2-[rac-(2S,5S)-2-(2-ethylpyrazol-3-yl)-5-methyl-1-piperidyl]acetate

2,2,2-Trifluoroethyl 2-chloro-2-oxo-acetate (343.50 mg, 1.80 mmol) was added dropwise to a stirred solution of rac-(2S,5S)-2-(2-ethylpyrazol-3-yl)-5-methyl-piperidine (0.4 g, 1.50 mmol, 2HCl) and TEA (532.16 mg, 5.26 mmol, 733.00 μL) in THF (30 mL) at 0° C., stirred for 1 hr at 0° C. Reaction mixture was used in the next step.

LCMS(ESI): [M+H]⁺ m/z: calcd 348.0; found 348.0; Rt=1.398 min.

### Step 4: The Synthesis of 2-Oxo-2-[rac-(2S,5S)-2-(2-ethylpyrazol-3-yl)-5-methyl-1-piperidyl]acetamide

Ammonia (696.26 mg, 40.88 mmol) was bubbled trough a reaction mixture at 20° C., stirred for 1 hr at 20° C. and 8 hr at 25° C. Reaction mixture was filtered, solid washed with THF (2×5 mL), filtrate concentrated in vacuo to give pure 2-oxo-2-[rac-(2S,5S)-2-(2-ethylpyrazol-3-yl)-5-methyl-1-piperidyl]acetamide (0.15 g, 567.49 μmol, 27.76% yield).

LCMS(ESI): [M+H]⁺ m/z: calcd 265.2; found 265.2; Rt=0.970 min.

### Step 5: The Synthesis of N-(4-Amino-1-tetrahydropyran-2-yl-pyrazolo[4,3-c]pyridin-7-yl)-2-oxo-2-[rac-(2S,5S)-2-(2-ethylpyrazol-3-yl)-5-methyl-1-piperidyl]acetamide

Copper (1.80 mg, 28.37 μmol), Copper (I) iodide (54.04 mg, 283.74 μmol, 9.62 μL), caesium carbonate (277.35 mg, 851.23 μmol) was added to a stirred solution of 2-oxo-2-[rac-(2S,5S)-2-(2-ethylpyrazol-3-yl)-5-methyl-1-piperidyl]acetamide (150.00 mg, 567.49 μmol), 7-bromo-1-tetrahydropyran-2-yl-pyrazolo[4,3-c]pyridin-4-amine (185.49 mg, 624.24 μmol), rac-(1R,2R)—N1,N2-dimethylcyclohexane-1,2-diamine (40.36 mg, 283.74 μmol) in 1,4-dioxane (7 mL) under Ar atmosphere and stirred at 110° C. for 48 hr in closed vial. Reaction mixture was filtered, solid washed with dioxane (2×3 mL), filtrate concentrated to give crude N-(4-amino-1-tetrahydropyran-2-yl-pyrazolo[4,3-c]pyridin-7-yl)-2-oxo-2-[rac-(2S,5S)-2-(2-ethylpyrazol-3-yl)-5-methyl-1-piperidyl]acetamide (0.27 g, 561.84 μmol, 99.01% yield).

LCMS(ESI): [M+H]⁺ m/z: calcd 481.2; found 481.2; Rt=1.086 min.

### Step 6: The Synthesis of N-(4-Amino-1H-pyrazolo[4,3-c]pyridin-7-yl)-2-oxo-2-[rac-(2S,5S)-2-(2-ethylpyrazol-3-yl)-5-methyl-1-piperidyl]acetamide

Hydrogen chloride solution 4.0M in dioxane (2.05 g, 56.18 mmol, 2.56 mL) was added to a solution of N-(4-amino-1-tetrahydropyran-2-yl-pyrazolo[4,3-c]pyridin-7-yl)-2-oxo-2-[rac-(2S,5S)-2-(2-ethylpyrazol-3-yl)-5-methyl-1-piperidyl]acetamide (270.00 mg, 561.84 μmol) in Methanol and stirred at 20° C. for 18 hr. Volatiles was evaporated in vacuo, the residue titered with IPA (5 mL), filtered, washed with IPA (5 mL), and submitted to HPLC (column: Chromatorex 18 SMB100-5T 100×19 mm 5 um; 5-5-25% 0-1-6 min H₂O/ACN/0.1% FA, A, flow rate: 30 mL/min) to give N-(4-amino-1H-pyrazolo[4,3-c]pyridin-7-yl)-2-oxo-2-[rac-(2S,5S)-2-(2-ethylpyrazol-3-yl)-5-methyl-1-piperidyl]acetamide (43 mg, 108.46 μmol, 19.31% yield).

LCMS(ESI): [M+H]⁺ m/z: calcd 397.2; found 397.2; Rt=2.248 min.

### Step 7: The Synthesis of N-(4-Amino-1H-pyrazolo[4,3-c]pyridin-7-yl)-2-oxo-2-[rac-(2S,5S)-2-(2-ethylpyrazol-3-yl)-5-methyl-1-piperidyl]acetamide (Compound 211) and N-(4-Amino-1H-pyrazolo[4,3-c]pyridin-7-yl)-2-oxo-2-[rac-(2R,5R)-2-(2-ethylpyrazol-3-yl)-5-methyl-1-piperidyl]acetamide (Compound 285)

Racemic N-(4-amino-1H-pyrazolo[4,3-c]pyridin-7-yl)-2-oxo-2-[rac-(2S,5S)-2-(2-ethylpyrazol-3-yl)-5-methyl-1-piperidyl]acetamide (43.0 mg, 108.46 μmol) was subjected to HPLC (Column: CHIRALPAK IC (250×20 mm, 5 mkm)-II; Mobile Phase: Hexane (0.1% DEA):MeOH:IPA, 70:15:15; Flow Rate: 12 mL/min; Injection Volume: 500 mkl; Rt1 (Compound 211)=24.488 min, Rt2 (Compound 285)=30.437 min) affording N-(4-amino-1H-pyrazolo[4,3-c]pyridin-7-yl)-2-oxo-2-[rac-(2S,5S)-2-(2-ethylpyrazol-3-yl)-5-methyl-1-piperidyl]acetamide (14.8 mg, 34.42% yield) and N-(4-amino-1H-pyrazolo[4,3-c]pyridin-7-yl)-2-oxo-2-[rac-(2R,5R)-2-(2-ethylpyrazol-3-yl)-5-methyl-1-piperidyl]acetamide (15.8 mg, 36.74% yield).

### Compound 285:

Preparative: RT (Column: CHIRALPAK IC (250×20 mm, 5 mkm)-II; Mobile Phase: Hexane (0.1% DEA):MeOH:IPA, 70:15:15; Flow Rate: 12 mL/min)=34.353 min.

Analytical: RT (Column: Chiralpak IC (250×4.6 mm, 5 mkm)-1; Mobile Phase: Hexane (0.1% EDA):IPA:MeOH, 70:15:15; Flow Rate: 0.6 mL/min)=30.437 min.

¹H NMR (600 MHz, dmso) δ 0.70-0.91 (m, 3H), 1.13-1.32 (m, 3H), 1.44-1.83 (m, 3H), 1.85-2.09 (m, 2H), 2.60-2.89 (m, 1H), 3.40-4.22 (m, 3H), 5.41-5.83 (m, 1H), 6.07-6.49 (m, 1H), 6.59-6.90 (m, 2H), 7.24-7.42 (m, 1H), 7.44-7.69 (m, 1H), 8.09-8.24 (m, 1H), 9.61-10.64 (m, 1H), 12.71-13.41 (m, 1H).

LCMS(ESI): [M+H]⁺ m/z: calcd 397.2; found 397.2; Rt=2.172 min.

### Compound 211:

Preparative: RT (Column: CHIRALPAK IC (250×20 mm, 5 mkm)-II; Mobile Phase:

Hexane (0.1% DEA):MeOH:IPA, 70:15:15; Flow Rate: 12 mL/min)=26.851 min.

Analytical: RT (Column: Chiralpak IC (250×4.6 mm, 5 mkm)-1; Mobile Phase: Hexane (0.1% EDA):IPA:MeOH, 70:15:15; Flow Rate: 0.6 mL/min)=24.488 min.

¹H NMR (600 MHz, dmso) δ 0.70-0.91 (m, 3H), 1.11-1.30 (m, 3H), 1.50-2.11 (m, 4H), 2.61-3.07 (m, 1H), 3.38-4.36 (m, 4H), 5.39-5.85 (m, 1H), 6.10-6.47 (m, 1H), 6.62-6.85 (m, 2H), 7.22-7.43 (m, 1H), 7.44-7.68 (m, 1H), 8.10-8.25 (m, 1H), 9.60-10.61 (m, 1H), 12.69-13.38 (m, 1H).

LCMS(ESI): [M+H]⁺ m/z: calcd 397.2; found 397.2; Rt=2.170 min.

### Example 189. N-(4-Amino-1H-pyrazolo[4,3-c]pyridin-7-yl)-2-oxo-2-[rac-(2R,5S)-2-[2-[4-(dimethylamino)cyclohexyl]-1,3-benzothiazol-5-yl]-5-methyl-1-piperidyl]acetamide (Compound 212)

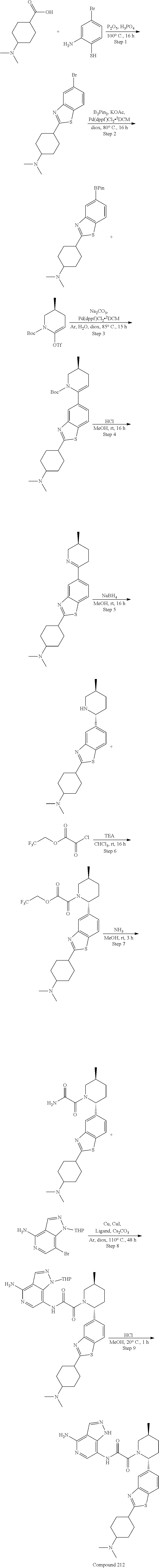

### Step 1: The Synthesis of 4-(5-Bromo-1,3-benzothiazol-2-yl)-N,N-dimethyl-cyclohexanamine

The stirred solution of Phosphorus (V) pentoxide (27.82 g, 195.99 mmol, 12.10 mL) in Phosphoric acid (19.21 g, 195.99 mmol, 11.30 mL) at 100° C. were added 4-(dimethylamino)cyclohexanecarboxylic acid (4 g, 23.36 mmol) and 2-amino-4-bromo-benzenethiol (4 g, 19.60 mmol). The resulting mixture was allowed to stir at 100° C. for 16 h. Upon completion, the reaction mixture was quenched with water (200 mL) and neutralized by NaOH to pH=8. The aqueous phase was extracted with EtOAc (2*100 mL). The combined organic phase was dried over Na₂SO₄ and concentrated under reduced pressure. The reaction was successful. The desired product 4-(5-bromo-1,3-benzothiazol-2-yl)-N,N-dimethyl-cyclohexanamine (5 g, 14.74 mmol, 75.19% yield) was isolated.

LCMS(ESI): [M+H]⁺ m/z: calcd 339.0; found 339.0; Rt=0.903 min.

### Step 2: The Synthesis of N,N-Dimethyl-4-[5-(4,4,5,5-tetramethyl-1,3,2-dioxaborolan-2-yl)-1,3-benzothiazol-2-yl]cyclohexanamine

To a stirred solution of 4-(5-bromo-1,3-benzothiazol-2-yl)-N,N-dimethyl-cyclohexanamine (5 g, 14.74 mmol) and 4,4,5,5-tetramethyl-2-(4,4,5,5-tetramethyl-1,3,2-dioxaborolan-2-yl)-1,3,2-dioxaborolane (3.8 g, 14.96 mmol) in DiOX (100 mL) was added Pd(dppf)Cl₂ (0.3 g, 362.88 μmol). The resulting suspension was degassed with argon at 50° C. for 0.5 h. Potassium Acetate (3 g, 30.57 mmol, 1.91 mL) was added. The reaction mixture was stirred at 80° C. for 16 h. Upon completion, the reaction mixture was evaporated, the crude product was quenched with water (200 mL). The aqueous phase was extracted with CHCl₃ (2*100 mL). The combined organic phase was dried over Na₂SO₄ and concentrated under reduced pressure. The reaction was successful. The desired product N,N-dimethyl-4-[5-(4,4,5,5-tetramethyl-1,3,2-dioxaborolan-2-yl)-1,3-benzothiazol-2-yl]cyclohexanamine (5.5 g, 14.24 mmol, 96.60% yield) was isolated.

LCMS(ESI): [M+H]⁺ m/z: calcd 387.0; found 387.0; Rt=1.144 min.

### Step 3: The Synthesis of tert-Butyl rac-(3S)-6-[2-[4-(dimethylamino)cyclohexyl]-1,3-benzothiazol-5-yl]-3-methyl-3,4-dihydro-2H-pyridine-1-carboxylate

To a stirred solution of N,N-dimethyl-4-[5-(4,4,5,5-tetramethyl-1,3,2-dioxaborolan-2-yl)-1,3-benzothiazol-2-yl]cyclohexanamine (2 g, 5.18 mmol) and tert-butyl rac-(3S)-3-methyl-6-(trifluoromethylsulfonyloxy)-3,4-dihydro-2H-pyridine-1-carboxylate (2 g, 5.79 mmol) in Water (10 mL) and DiOX (20 mL) was added Sodium carbonate (1.10 g, 10.35 mmol, 433.38 μL). The resulting suspension was degassed with argon at 50° C. for 0.5 h. Pd(dppf)Cl2 (0.2 g, 241.92 μmol) was added. The reaction mixture was stirred at 80° C. for 16 h. Upon completion, the reaction mixture was concentrated under reduced pressure, dissolved in CHCl₃, dried over Na₂SO₄, filtered and the filtrate was evaporated. The reaction was successful. The desired product tert-butyl rac-(3S)-6-[2-[4-(dimethylamino)cyclohexyl]-1,3-benzothiazol-5-yl]-3-methyl-3,4-dihydro-2H-pyridine-1-carboxylate (2.3 g, 5.05 mmol, 97.51% yield) was isolated.

LCMS(ESI): [M+H]⁺ m/z: calcd 456.4; found 456.4; Rt=0.874 min.

### Step 4: The Synthesis of N,N-Dimethyl-4-[5-[rac-(3S)-3-methyl-2,3,4,5-tetrahydropyridin-6-yl]-1,3-benzothiazol-2-yl]cyclohexanamine

To the stirred solution of tert-butyl rac-(3S)-6-[2-[4-(dimethylamino)cyclohexyl]-1,3-benzothiazol-5-yl]-3-methyl-3,4-dihydro-2H-pyridine-1-carboxylate (2.3 g, 5.05 mmol) in MeOH (30 mL) was added DiOX/HCl (10 mL). The resulting mixture was allowed to stir at 25° C. for 16 h. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The reaction was successful. The desired product N,N-dimethyl-4-[5-[rac-(3S)-3-methyl-2,3,4,5-tetrahydropyridin-6-yl]-1,3-benzothiazol-2-yl]cyclohexanamine (1.7 g, 4.78 mmol, 94.73% yield) was isolated.

LCMS(ESI): [M+H]⁺ m/z: calcd 356.1; found 356.1; Rt=0.727 min.

### Step 5: The Synthesis of N,N-Dimethyl-4-[5-[rac-(2R,5S)-5-methyl-2-piperidyl]-1,3-benzothiazol-2-yl]cyclohexanamine

To a stirred solution of N,N-dimethyl-4-[5-[rac-(3S)-3-methyl-2,3,4,5-tetrahydropyridin-6-yl]-1,3-benzothiazol-2-yl]cyclohexanamine (1.7 g, 4.78 mmol) in MeOH (30 mL) was added Sodium Borohydride (0.3 g, 7.93 mmol, 279.33 μL) at 5° C. The resulting reaction mixture was stirred at 25° C. for 16 h. Upon completion, the reaction mixture was evaporated, quenched with water (20 mL). The aqueous phase was extracted with CHCl₃ (2*20 mL). The combined organic phase was dried over Na₂SO₄ and concentrated under reduced pressure. The reaction was successful. The desired product N,N-dimethyl-4-[5-[rac-(2R,5S)-5-methyl-2-piperidyl]-1,3-benzothiazol-2-yl]cyclohexanamine (1.5 g, 4.20 mmol, 87.74% yield) was isolated.

LCMS(ESI): [M+H]⁺ m/z: calcd 358.2; found 358.2; Rt=0.771 min.

### Step 6: The Synthesis of 2,2,2-Trifluoroethyl 2-oxo-2-[rac-(2R,5S)-2-[2-[4-(dimethylamino)cyclohexyl]-1,3-benzothiazol-5-yl]-5-methyl-1-piperidyl]acetate

To a solution of N,N-dimethyl-4-[5-[rac-(2R,5S)-5-methyl-2-piperidyl]-1,3-benzothiazol-2-yl]cyclohexanamine (0.5 g, 1.40 mmol) and Triethylamine (217.80 mg, 2.15 mmol, 0.3 mL) in CHCl₃ (10 mL) was added 2,2,2-trifluoroethyl 2-chloro-2-oxo-acetate (0.3 g, 1.57 mmol) at 5° C. The resulting reaction mixture was allowed to warm to room temperature and stirred for 16 h at 25° C. After 16 hr the reaction mixture was quenched with water (20 mL) and extracted with CHCl₃ (2*10 mL). The organic layer was dried over Na₂SO₄ and evaporated under reduced pressure. The reaction was successful. The desired product 2,2,2-trifluoroethyl 2-oxo-2-[rac-(2R,5S)-2-[2-[4-(dimethylamino)cyclohexyl]-1,3-benzothiazol-5-yl]-5-methyl-1-piperidyl]acetate (0.7 g, 1.37 mmol, 97.85% yield) was isolated.

LCMS(ESI): [M+H]⁺ m/z: calcd 512.2; found 512.2; Rt=0.892 min.

### Step 7: The Synthesis of 2-Oxo-2-[rac-(2R,5S)-2-[2-[4-(dimethylamino)cyclohexyl]-1,3-benzothiazol-5-yl]-5-methyl-1-piperidyl]acetamide

To a stirred solution of 2,2,2-trifluoroethyl 2-oxo-2-[rac-(2R,5S)-2-[2-[4-(dimethylamino)cyclohexyl]-1,3-benzothiazol-5-yl]-5-methyl-1-piperidyl]acetate (0.7 g, 1.37 mmol) in MeOH (10 mL) was added MeOH/NH₃ (5 mL). The resulting reaction mixture was stirred at 25° C. for 3 h. Upon completion, the reaction mixture was concentrated under reduced pressure. The obtained crude product was purified by reverse phase HPLC chromatography ((Mobile Phase, Column): SYSTEM 50-100% 0-5 min H₂O/MeOH/0.1% NH₄OH, flow: 30 mL/min (loading pump 4 mL/min methanol); column: XBridge BEH C18 100×19 mm, 5 um) to afford product 2-oxo-2-[rac-(2R,5S)-2-[2-[4-(dimethylamino)cyclohexyl]-1,3-benzothiazol-5-yl]-5-methyl-1-piperidyl]acetamide (0.2 g, 466.65 μmol, 34.11% yield).

LCMS(ESI): [M+H]⁺ m/z: calcd 429.2; found 429.2; Rt=2.432 min.

### Step 8: The Synthesis of N-(4-Amino-1-tetrahydropyran-2-yl-pyrazolo[4,3-c]pyridin-7-yl)-2-oxo-2-[rac-(2R,5S)-2-[2-[4-(dimethylamino)cyclohexyl]-1,3-benzothiazol-5-yl]-5-methyl-1-piperidyl]acetamide

Copper (1.48 mg, 23.33 μmol), Copper (I) iodide (44.44 mg, 233.32 μmol, 7.91 μL), caesium carbonate (228.06 mg, 699.97 μmol) was added to a stirred solution of 2-oxo-2-[rac-(2R,5S)-2-[2-[4-(dimethylamino)cyclohexyl]-1,3-benzothiazol-5-yl]-5-methyl-1-piperidyl]acetamide (0.2 g, 466.65 μmol), 7-bromo-1-tetrahydropyran-2-yl-pyrazolo[4,3-c]pyridin-4-amine (145.60 mg, 489.98 μmol), rac-(1R,2R)—N1,N2-dimethylcyclohexane-1,2-diamine (33.19 mg, 233.32 μmol) in 1,4-dioxane (7 mL) under Ar atmosphere and stirred at 110° C. for 48 hr in closed vial. Reaction mixture was filtered, solid washed with dioxane (2×3 mL), filtrate concentrated to give crude N-(4-amino-1-tetrahydropyran-2-yl-pyrazolo[4,3-c]pyridin-7-yl)-2-oxo-2-[rac-(2R,5S)-2-[2-[4-(dimethylamino)cyclohexyl]-1,3-benzothiazol-5-yl]-5-methyl-1-piperidyl]acetamide (0.3 g, 465.24 μmol, 99.70% yield).

LCMS(ESI): [M+2H]⁺ m/z: calcd 646.2; found 646.2; Rt=1.007 min.

### Step 9: The Synthesis of N-(4-Amino-1H-pyrazolo[4,3-c]pyridin-7-yl)-2-oxo-2-[rac-(2R,5S)-2-[2-[4-(dimethylamino)cyclohexyl]-1,3-benzothiazol-5-yl]-5-methyl-1-piperidyl]acetamide (Compound 212)

Hydrogen chloride solution 4.0M in dioxane (848.13 mg, 23.26 mmol, 1.06 mL) was added to a solution of N-(4-amino-1-tetrahydropyran-2-yl-pyrazolo[4,3-c]pyridin-7-yl)-2-oxo-2-[rac-(2R,5S)-2-[2-[4-(dimethylamino)cyclohexyl]-1,3-benzothiazol-5-yl]-5-methyl-1-piperidyl]acetamide (0.3 g, 465.24 μmol) in Methanol (2.09 mL) and stirred at 20° C. for 1 hr. Volatiles was evaporated in vacuo, the residue triturred with IPA (5 mL), filtered, washed with IPA (5 mL), and submitted to HPLC (column: Chromatorex 18 SMB100-5T 100×19 mm 5 um; 5-5-40% 0-2-5 min H₂O/ACN/0.1% FA, flow rate: 30 mL/min) to give crude product (1 fraction). Obtained material was purified by HPLC (Chromatorex 18 SMB100-5T 100×19 mm 5 um; 0-25% 0-5 min H₂O/ACN/0.1% FA, flow rate: 30 mL/min) to give N-(4-amino-1H-pyrazolo[4,3-c]pyridin-7-yl)-2-oxo-2-[rac-(2R,5S)-2-[2-[4-(dimethylamino)cyclohexyl]-1,3-benzothiazol-5-yl]-5-methyl-1-piperidyl]acetamide (6 mg, 10.05 μmol, 2.16% yield, HCl).

¹H NMR (500 MHz, dmso) δ 0.16-1.11 (m, 3H), 1.31-1.48 (m, 3H), 1.51-1.98 (m, 8H), 2.10-2.22 (m, 3H), 2.25 (s, 6H), 3.01-3.09 (m, 2H), 3.75-4.15 (m, 1H), 5.09-5.81 (m, 1H), 6.53-7.10 (m, 2H), 7.33-7.57 (m, 1H), 7.59-7.78 (m, 1H), 7.84-8.01 (m, 1H), 8.01-8.32 (m, 3H), 9.59-10.71 (m, 1H), 12.53-13.46 (m, 1H).

LCMS(ESI): [M+H]⁺ m/z: calcd 561.2; found 561.2; Rt=2.102 min.

### Example 190. N-(4-Amino-1H-pyrazolo[4,3-c]pyridin-7-yl)-2-oxo-2-[rac-(2S)-2-(3-pyrrolidin-1-ylphenyl)-1-piperidyl]acetamide (Compound 213) and N-(4-Amino-1H-pyrazolo[4,3-c]pyridin-7-yl)-2-oxo-2-[rac-(2R)-2-(3-pyrrolidin-1-ylphenyl)-1-piperidyl]acetamide (Compound 271)

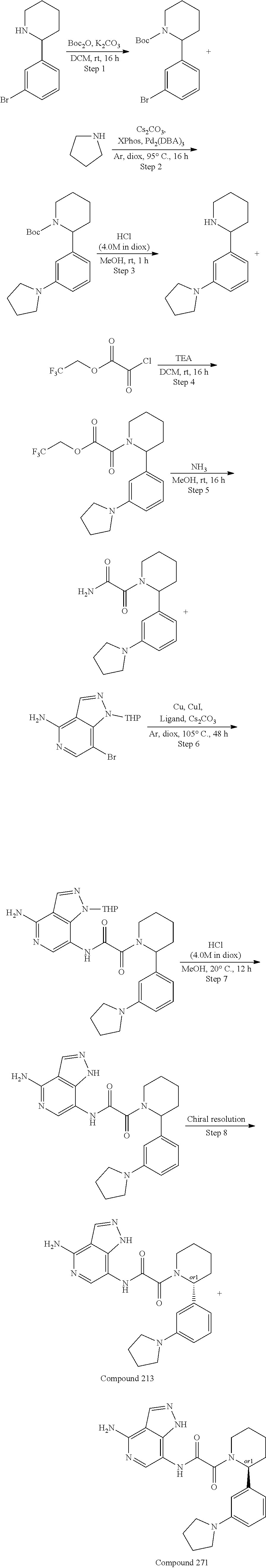

### Step 1: The Synthesis of tert-Butyl 2-(3-bromophenyl)piperidine-1-carboxylate

tert-Butoxycarbonyl tert-butyl carbonate (1.91 g, 8.74 mmol, 2.01 mL) was added dropwise to Potassium carbonate (1.73 g, 12.49 mmol, 753.96 μL) and 2-(3-bromophenyl)piperidine (2 g, 8.33 mmol) in DCM (47.24 mL) at rt. Water was added to the solution after overnight stirring at rt. Organic phase was separated, and dried over Na₂SO₄, filtered and concentrated to afford tert-butyl 2-(3-bromophenyl)piperidine-1-carboxylate (2.8 g, 8.23 mmol, 98.81% yield) as a light yellow oil.

LCMS(ESI): [M-Boc]⁺ m/z: calcd 240.0; found 240.0; Rt=1.733 min.

### Step 2: The Synthesis of tert-Butyl 2-(3-pyrrolidin-1-ylphenyl)piperidine-1-carboxylate

A mixture of tert-butyl 2-(3-bromophenyl)piperidine-1-carboxylate (0.5 g, 1.47 mmol), pyrrolidine (130.64 mg, 1.84 mmol, 152.62 μL) and Cesium carbonate (957.57 mg, 2.94 mmol) in dioxane (5 mL) was evacuated and then backfilled with argon. This operation was repeated three times, and then (9,9-Dimethyl-9H-xanthene-4,5-diyl)bis(diphenylphosphane) (51.02 mg, 88.17 μmol) and tris(1,5-diphenylpenta-1,4-dien-3-one)dipalladium(40.37 mg, 44.08 μmol) were added under argon and the reaction mixture was stirred under argon at 95° C. for 16 hr. The reaction mixture was cooled down and filtered through layer of SiO₂. The filtrate was concentrated in vacuo to give tert-butyl 2-(3-pyrrolidin-1-ylphenyl)piperidine-1-carboxylate (0.42 g, 1.27 mmol, 86.49% yield).

LCMS(ESI): [M+H]⁺ m/z: calcd 331.2; found 331.2; Rt=1.406 min.

### Step 3: The Synthesis of 2-(3-Pyrrolidin-1-ylphenyl)piperidine

diox/HCl (1.27 mmol, 5 mL) was added to a tert-butyl 2-(3-pyrrolidin-1-ylphenyl)piperidine-1-carboxylate (0.42 g, 1.27 mmol) at rt with vigorous stirring. After 1 hr, the reaction mixture was concentrated in vacuo. Water, DCM (20 ml) and NaHCO₃ were added. Organic phase separated, washed by water, dried over Na₂SO₄ and concentrated in vacuo to 2-(3-pyrrolidin-1-ylphenyl)piperidine (0.28 g, 1.22 mmol, 95.64% yield).

LCMS(ESI): [M+H]⁺ m/z: calcd 231.2; found 231.2; Rt=0.996 min.

### Step 4: The Synthesis of 2,2,2-Trifluoroethyl 2-oxo-2-[2-(3-pyrrolidin-1-ylphenyl)-1-piperidyl]acetate

2-(3-Pyrrolidin-1-ylphenyl)piperidine (0.28 g, 1.22 mmol) was dissolved in DCM (20 mL) and TEA (307.50 mg, 3.04 mmol, 423.56 μL) was added. The reaction mixture was cooled and 2,2,2-trifluoroethyl 2-chloro-2-oxo-acetate (277.88 mg, 1.46 mmol) was added dropwise. After that it was stirred at rt overnight. Then water (10 ml) was added, organic layer was stirred with Brine and dried over Na₂SO₄, evaporated in vacuum to afford 2,2,2-trifluoroethyl 2-oxo-2-[2-(3-pyrrolidin-1-ylphenyl)-1-piperidyl]acetate (0.45 g, 1.17 mmol, 96.31% yield).

LCMS(ESI): [M+H]⁺ m/z: calcd 385.1; found 385.1; Rt=1.608 min.

### Step 5: The Synthesis of 2-Oxo-2-[2-(3-pyrrolidin-1-ylphenyl)-1-piperidyl]acetamide

2,2,2-Trifluoroethyl 2-oxo-2-[2-(3-pyrrolidin-1-ylphenyl)-1-piperidyl]acetate (0.45 g, 1.17 mmol) was dissolved in NH₃/MeOH (10 mL) and stirred overnight at rt. Then it was evaporated in vacuum and subjected to CC (CHCl₃-MeCN was used as an eluent mixture) to afford 2-oxo-2-[2-(3-pyrrolidin-1-ylphenyl)-1-piperidyl]acetamide (0.2 g, 663.61 μmol, 56.69% yield).

LCMS(ESI): [M+H]⁺ m/z: calcd 302.2; found 302.2; Rt=1.240 min.

### Step 6: The Synthesis of N-(4-Amino-1-tetrahydropyran-2-yl-pyrazolo[4,3-c]pyridin-7-yl)-2-oxo-2-[2-(3-pyrrolidin-1-ylphenyl)-1-piperidyl]acetamide

7-Bromo-1-tetrahydropyran-2-yl-pyrazolo[4,3-c]pyridin-4-amine (384.52 mg, 1.29 mmol), 2-oxo-2-[2-(3-pyrrolidin-1-ylphenyl)-1-piperidyl]acetamide (0.3 g, 995.41 μmol), Cu (1.27 mg, 19.91 μmol), CuI (28.44 mg, 149.31 μmol, 5.06 μL), Cesium carbonate (486.49 mg, 1.49 mmol) and rac-(1R,2R)—N1,N2-dimethylcyclohexane-1,2-diamine (21.24 mg, 149.31 μmol) were mixed in dioxane (6 mL), purged with Ar for 15 minutes and then heated in the sealed tube at 105° C. for 48 hr. Final mixture was filtered and dioxane was evaporated in vacuo. The crude product 0.45 g was purified by RP-HPLC (column: XBridge C18 5 um 130 A; 50-100% 0-5 min H₂O/CH3OH/0.1% NH₄OH, flow: 30 ml/min) to give N-(4-amino-1-tetrahydropyran-2-yl-pyrazolo[4,3-c]pyridin-7-yl)-2-oxo-2-[2-(3-pyrrolidin-1-ylphenyl)-1-piperidyl]acetamide (65.70 mg, 126.93 μmol, 12.75% yield).

LCMS(ESI): [M+H]⁺ m/z: calcd 518.2; found 518.2; Rt=1.610 min.

### Step 7: The Synthesis of N-(4-Amino-1H-pyrazolo[4,3-c]pyridin-7-yl)-2-oxo-2-[2-(3-pyrrolidin-1-ylphenyl)-1-piperidyl]acetamide

Hydrogen chloride solution 4.0M in dioxane (3.20 g, 87.77 mmol, 4 mL) was added to a solution of N-(4-amino-1-tetrahydropyran-2-yl-pyrazolo[4,3-c]pyridin-7-yl)-2-oxo-2-[2-(3-pyrrolidin-1-ylphenyl)-1-piperidyl]acetamide (65.70 mg, 126.93 μmol) in MeOH (15 mL). The reaction mixture was stirred at 20° C. for 12 hr, then evaporated was purified by RP-HPLC (column: XBridge BEH18 SMB100-BT 100*19 mm; 30-80% 0-5 min H₂O/CH₃OH/0.1% NH₄OH, flow: 30 mL/min) to give N-(4-amino-1H-pyrazolo[4,3-c]pyridin-7-yl)-2-oxo-2-[2-(3-pyrrolidin-1-ylphenyl)-1-piperidyl]acetamide (27.10 mg, 62.51 μmol, 49.25% yield).

LCMS(ESI): [M+H]⁺ m/z: calcd 434.2; found 434.2; Rt=2.379 min.

### Step 8: The Synthesis of N-(4-Amino-1H-pyrazolo[4,3-c]pyridin-7-yl)-2-oxo-2-[rac-(2S)-2-(3-pyrrolidin-1-ylphenyl)-1-piperidyl]acetamide (Compound 213) and N-(4-Amino-1H-pyrazolo[4,3-c]pyridin-7-yl)-2-oxo-2-[rac-(2R)-2-(3-pyrrolidin-1-ylphenyl)-1-piperidyl]acetamide (Compound 271)

N-(4-Amino-1H-pyrazolo[4,3-c]pyridin-7-yl)-2-oxo-2-[2-(3-pyrrolidin-1-ylphenyl)-1-piperidyl]acetamide (27.10 mg, 62.51 μmol) was subjected to chiral HPLC purification (Column: Chiralpak IJ (250-20 mm-5 m); Mobile phase: MeOH-IPA, 50-50 Flow Rate: 10 mL/min) to give the two individual enantiomers Compound 213 N-(4-amino-1H-pyrazolo[4,3-c]pyridin-7-yl)-2-oxo-2-[rac-(2S)-2-(3-pyrrolidin-1-ylphenyl)-1-piperidyl]acetamide (0.00962 g, 22.19 μmol, 35.50% yield) and Compound 271 N-(6-amino-3-pyridyl)-2-[(2S,5R)-5-methyl-2-[2-(trifluoromethyl)-1,3-benzothiazol-5-yl]-1-piperidyl]-2-oxo-acetamide (0.05775 g, 124.60 μmol, 50.22% yield)

### Compound 213:

Preparative: RT (Column: CHIRALCEL OJ-H (250×20 mm, 5 mkm); Mobile Phase: IPA:MeOH, 50:50; Flow Rate: 12 mL/min)=11.902 min.

Analytical: RT (Column: Chiralcel OD-H (250×4.6 mm, 5 mkm)-1; Mobile Phase: Hexane (0.1% EDA):IPA:MeOH, 70:15:15; Flow Rate: 0.6 mL/min)=15.592 min.

¹H NMR (600 MHz, dmso) δ 1.43-1.83 (m, 4H), 1.88-1.95 (m, 4H), 2.41-2.44 (m, 1H), 2.60-2.77 (m, 1H), 3.07-3.27 (m, 5H), 3.60-4.38 (m, 1H), 5.17-5.77 (m, 1H), 6.16-6.49 (m, 2H), 6.53-6.60 (m, 1H), 6.60-6.92 (m, 2H), 7.03-7.29 (m, 1H), 7.39-7.80 (m, 1H), 8.06-8.36 (m, 1H), 9.47-10.57 (m, 1H), 12.51-13.41 (m, 1H).

LCMS(ESI): [M+H]⁺ m/z: calcd 434.2; found 434.2; Rt=2.720 min.

### Compound 271:

Preparative: RT (Column: CHIRALCEL OJ-H (250×20 mm, 5 mkm); Mobile Phase: IPA:MeOH, 50:50; Flow Rate: 12 mL/min)=31.038 min.

Analytical: RT (Column: Chiralcel OD-H (250×4.6 mm, 5 mkm)-1; Mobile Phase: Hexane (0.1% EDA):IPA:MeOH, 70:15:15; Flow Rate: 0.6 mL/min)=12.302 min.

¹H NMR (600 MHz, dmso) δ 1.24-1.82 (m, 4H), 1.83-2.02 (m, 4H), 2.40-2.44 (m, 1H), 2.61-2.74 (m, 1H), 3.03-3.28 (m, 5H), 3.63-4.39 (m, 1H), 5.19-5.73 (m, 1H), 6.15-6.46 (m, 2H), 6.49-6.61 (m, 1H), 6.62-7.08 (m, 2H), 7.11-7.37 (m, 1H), 7.49-7.80 (m, 1H), 8.10-8.22 (m, 1H), 9.49-10.53 (m, 1H), 12.54-13.41 (m, 1H).

LCMS(ESI): [M+H]⁺ m/z: calcd 434.2; found 434.2; Rt=2.720 min.

### Example 191. N-(4-Amino-1H-pyrazolo[4,3-c]pyridin-7-yl)-2-oxo-2-[rac-(2R,5S)-5-methyl-2-(2-methyl-7-quinolyl)-1-piperidyl]acetamide (Compound 215)

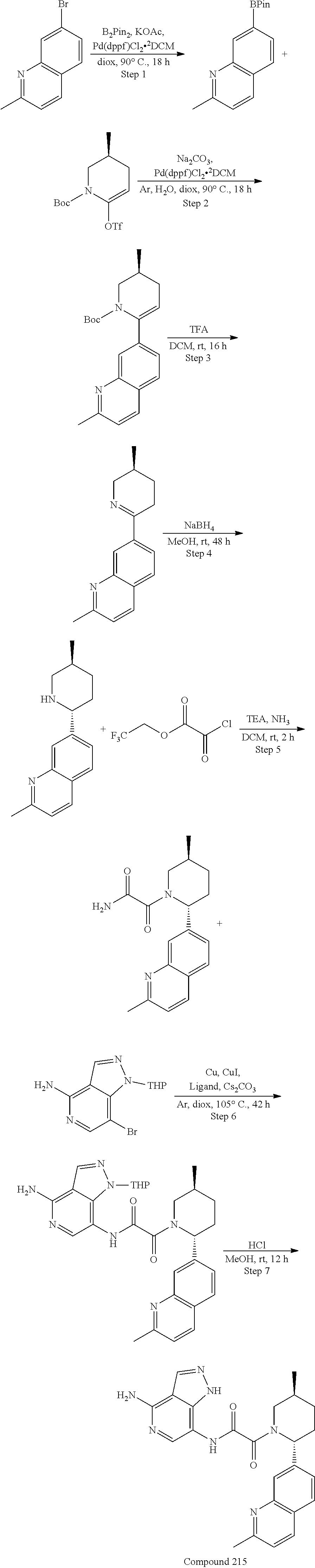

### Step 1: The Synthesis of 2-Methyl-7-(4,4,5,5-tetramethyl-1,3,2-dioxaborolan-2-yl)quinoline

A mixture of 7-bromo-2-methyl-quinoline (3 g, 13.51 mmol), 4,4,5,5-tetramethyl-2-5 (4,4,5,5-tetramethyl-1,3,2-dioxaborolan-2-yl)-1,3,2-dioxaborolane (3.77 g, 14.86 mmol) and Potassium Acetate (3.31 g, 33.77 mmol, 2.11 mL) in 1,4-dioxane (100 mL) was evacuated and then backfilled with argon. This operation was repeated two times, then Pd(dppf)Cl₂·CH₂Cl₂ (1.10 g, 1.35 mmol) was added and the reaction mixture was stirred under argon at 90° C. for 18 hr. The LCMS of the aliquot showed complete conversion of starting bromide. The reaction mixture contained crude 2-methyl-7-(4,4,5,5-tetramethyl-1,3,2-dioxaborolan-2-yl)quinoline (3.64 g, crude) (yield based on 1H NMR and LCMS) was used directly in the next step.

LCMS(ESI): [M+H]⁺ m/z: calcd 270.2; found 270.2; Rt=1.142 min.

### Step 2: The Synthesis of tert-Butyl rac-(3S)-3-methyl-6-(2-methyl-7-quinolyl)-3,4-dihydro-2H-pyridine-1-carboxylate

tert-Butyl rac-(3S)-3-methyl-6-(trifluoromethylsulfonyloxy)-3,4-dihydro-2H-pyridine-1-carboxylate (5.14 g, 14.88 mmol), Sodium carbonate (4.30 g, 40.57 mmol, 1.70 mL) and water (20 mL) were added to a solution of crude from previous step 2-methyl-7-(4,4,5,5-tetramethyl-1,3,2-dioxaborolan-2-yl)quinoline (3.64 g, 13.52 mmol) in 1,4-dioxane (100 mL). The resulting mixture was evacuated and then backfilled with argon, this operation was repeated three times, then Pd(dppf)Cl₂·DCM (552.22 mg, 676.21 μmol) was added under argon. The reaction mixture was stirred under argon at 90° C. for 18 hr. The reaction mixture was cooled and then filtered. The filtercake was additionally washed with 1,4-dioxane (2*25 mL) and discarded. The combined filtrate was concentrated in vacuo to afford crude product 28% purity by LCMS (15 g), which was purified by column chromatography on silica gel using hexane/MTBE gradient (0-100% MTBE) to afford 55% purity product tert-butyl rac-(3S)-3-methyl-6-(2-methyl-7-quinolyl)-3,4-dihydro-2H-pyridine-1-carboxylate (1.5 g, 4.43 mmol, 32.77% yield) as yellow gum, which was used directly in the next step.

LCMS(ESI): [M+H]⁺ m/z: calcd 339.2; found 339.2; Rt=1.027 min.

### Step 3: The Synthesis of 2-Methyl-7-[rac-(3S)-3-methyl-2,3,4,5-tetrahydropyridin-6-yl]quinoline

tert-Butyl rac-(3S)-3-methyl-6-(2-methyl-7-quinolyl)-3,4-dihydro-2H-pyridine-1-carboxylate (1.5 g, 4.43 mmol) was mixed with TFA (0.6 mL) and stirred at room temperature overnight. Then, the volatiles were removed under reduced pressure to afford 2-methyl-7-[rac-(3S)-3-methyl-2,3,4,5-tetrahydropyridin-6-yl]quinoline (1.06 g, 3.36 mmol, 75.74% yield).

### Step 4: The Synthesis of 2-Methyl-7-[rac-(2R,5S)-5-methyl-2-piperidyl]quinoline

Sodium Borohydride (126.99 mg, 3.36 mmol) was added to the stirred solution of 2-methyl-7-[rac-(3S)-3-methyl-2,3,4,5-tetrahydropyridin-6-yl]quinoline (0.8 g, 3.36 mmol) in MeOH (30 mL). The obtained mixture was stirred at room temperature for 48 hr. Then, the volatiles were removed under reduced pressure and the residue was mixed with water (20 mL). The product was extracted with DCM (3:10 mL). The combined organic layer was dried over Na₂SO₄, filtered and evaporated under reduced pressure to obtain 2-methyl-7-[rac-(2R,5S)-5-methyl-2-piperidyl]quinoline (0.52 g, 806.77 mg, 64.45%).

### Step 5: The Synthesis of 2-Oxo-2-[rac-(2R,5S)-5-methyl-2-(2-methyl-7-quinolyl)-1-piperidyl]acetamide

To a solution of 2-methyl-7-[rac-(2R,5S)-5-methyl-2-piperidyl]quinoline (0.52 g, 2.16 mmol) and Triethylamine (437.86 mg, 4.33 mmol, 603.12 μL) in THF (15 mL), 2,2,2-trifluoroethyl 2-chloro-2-oxo-acetate (618.26 mg, 3.25 mmol) was added portionwise at 25° C. The resulting mixture was stirred for 2 hr. LCMS showed full conversion of SM. Through resulting mixture Ammonia (36.85 mg, 2.16 mmol) was bubbled during 20 min at 25° C. The formed precipitate was filtered off, washed with THF (30 mL) and the solvent was evaporated in vacuo to give 2-oxo-2-[rac-(2R,5S)-5-methyl-2-(2-methyl-7-quinolyl)-1-piperidyl]acetamide (0.5 g, crude).

### Step 6: The Synthesis of N-(4-Amino-2-tetrahydropyran-2-yl-pyrazolo[4,3-c]pyridin-7-yl)-2-oxo-2-[rac-(2R,5S)-5-methyl-2-(2-methyl-7-quinolyl)-1-piperidyl]acetamide

A mixture of 2-oxo-2-[rac-(2R,5S)-5-methyl-2-(2-methyl-7-quinolyl)-1-piperidyl]acetamide (160 mg, 513.85 μmol), 7-bromo-2-tetrahydropyran-2-yl-pyrazolo[4,3-c]pyridin-4-amine (229.03 mg, 770.77 μmol), copper (5 mg, 78.68 μmol), Copper (I) iodide (120 mg, 630.09 μmol, 21.35 μL), caesium carbonate (251.13 mg, 770.77 μmol) and rac-(1R,2R)—N1,N2-dimethylcyclohexane-1,2-diamine (120 mg, 843.64 μmol) in 1,4-dioxane (6.00 mL) was stirred in a sealed vial under argon at 105° C. for 42 hr. The resulting mixture was cooled down and filtered. The filtercake was washed successively with THF (2*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-2-oxo-2-[rac-(2R,5S)-5-methyl-2-(2-methyl-7-quinolyl)-1-piperidyl]acetamide (530 mg, crude) as brown gum, which was used directly in the next step.

LCMS(ESI): [M+H]⁺ m/z: calcd 528.2; found 528.2; Rt=2.370 min.

### Step 7: The Synthesis of N-(4-Amino-1H-pyrazolo[4,3-c]pyridin-7-yl)-2-oxo-2-[rac-(2R,5S)-5-methyl-2-(2-methyl-7-quinolyl)-1-piperidyl]acetamide (Compound 215)

Hydrogen chloride solution 4.0M in dioxane (3.42 g, 13.06 mmol, 4.28 mL, 13.9% purity) was added to a stirred solution of crude from previous step N-(4-amino-2-tetrahydropyran-2-yl-pyrazolo[4,3-c]pyridin-7-yl)-2-oxo-2-[rac-(2R,5S)-5-methyl-2-(2-methyl-7-quinolyl)-1-piperidyl]acetamide (530 mg, 1.00 mmol) in methanol (4.97 mL) at 25° C. The resulting solution was stirred at 25° C. for 12 hr, then concentrated to dryness in vacuo and the residue was submitted to reverse phase HPLC (column: Chromatorex 18 SMB100-5T 100×19 mm 5 um; mobile phase: 5-30% 0-5 min H₂O/Acetonitrile/0.1% FA; flow: 30 mL/min (loading pump 4 mL/min water)) to afford Compound 215 N-(4-amino-1H-pyrazolo[4,3-c]pyridin-7-yl)-2-oxo-2-[rac-(2R,5S)-5-methyl-2-(2-methyl-7-quinolyl)-1-piperidyl]acetamide (40 mg, 81.71 μmol, 8.13% yield, HCOOH) as yellow solid.

¹H NMR (500 MHz, dmso) δ 0.82-1.12 (m, 3H), 1.31-1.45 (m, 1H), 1.49-1.79 (m, 2H), 1.81-2.08 (m, 2H), 2.09-2.25 (m, 1H), 2.61-2.70 (m, 3H), 3.81-4.15 (m, 1H), 5.16-5.89 (m, 1H), 6.57-7.00 (m, 2H), 7.35-7.44 (m, 1H), 7.47-7.59 (m, 1H), 7.60-7.77 (m, 1H), 7.82-8.08 (m, 2H), 8.14-8.25 (m, 2H), 9.60-10.73 (m, 1H), 12.39-13.52 (m, 1H).

LCMS(ESI): [M+H]⁺ m/z: calcd 444.2; found 444.2; Rt=1.341 min.

### Example 192. N-(4-Amino-1H-pyrazolo[4,3-c]pyridin-7-yl)-2-oxo-2-[rac-(2R,5R)-4,4-difluoro-5-methyl-2-phenyl-1-piperidyl]acetamide (Compound 216)

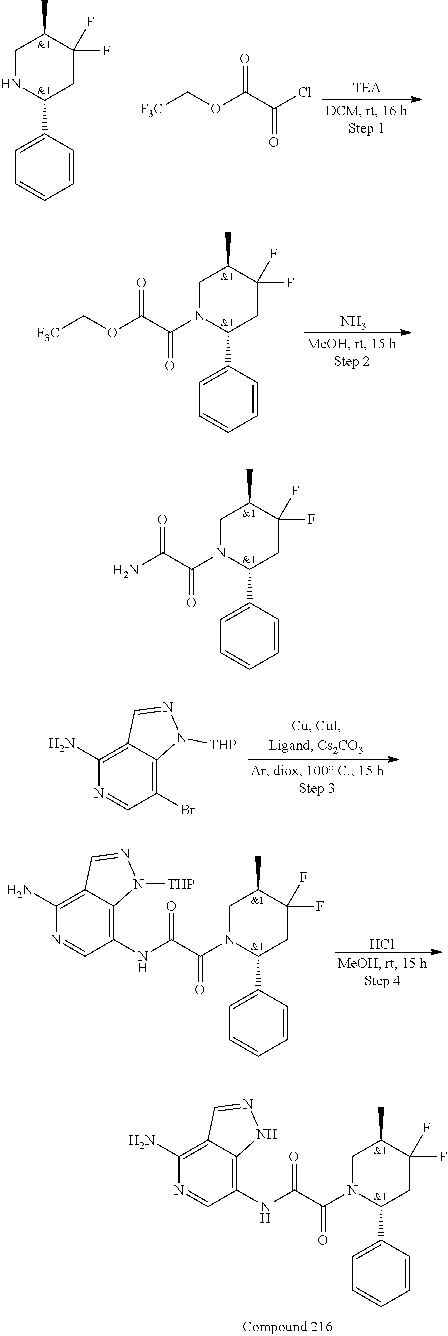

### Step 1: The Synthesis of 2,2,2-Trifluoroethyl 2-oxo-2-[rac-(2R,5R)-4,4-difluoro-5-methyl-2-phenyl-1-piperidyl]acetate

To a stirred solution of rac-(2R,5R)-4,4-difluoro-5-methyl-2-phenyl-piperidine (530 mg, 2.51 mmol) in Dichloromethane (10 mL) were added triethylamine (380.81 mg, 3.76 mmol, 524.53 μL) respectively at room temperature. The resulting reaction mixture was cooled to 0° C. Then 2,2,2-trifluoroethyl 2-chloro-2-oxo-acetate (477.95 mg, 2.51 mmol) was added dropwise. The reaction was stirred extra 30 minutes at 0° C., then allowed warmed to room temperature and stirred 15 hr. Upon completion, the reaction mixture was washed with water (2×20 mL). Organic phase was then dried over Na₂SO₄, filtered and concentrated under reduced pressure to obtain crude product as brown gum. Product was used for the next step without purification. The reaction was successful. The desired product 2,2,2-trifluoroethyl 2-oxo-2-[rac-(2R,5R)-4,4-difluoro-5-methyl-2-phenyl-1-piperidyl]acetate (814 mg, crude) was isolated as a brown gum.

LCMS(ESI): [M+H]⁺ m/z: calcd 366.0; found 366.0; Rt=1.302 min.

### Step 2: The Synthesis of 2-Oxo-2-[rac-(2R,5R)-4,4-difluoro-5-methyl-2-phenyl-1-piperidyl]acetamide

A solution of 2,2,2-trifluoroethyl 2-oxo-2-[rac-(2R,5R)-4,4-difluoro-5-methyl-2-phenyl-1-piperidyl]acetate (814 g, 2.23 mol) in Methanol/NH₃ (7N) (5 mL) was stirred at 25° C. for 15 hr. The solvent was evaporated to obtain 2-oxo-2-[rac- (2R,5R)-4,4-difluoro-5-methyl-2-phenyl-1-piperidyl]acetamide (453 mg, crude).

### Step 3: The Synthesis of N-(4-Amino-2-tetrahydropyran-2-yl-pyrazolo[4,3-c]pyridin-7-yl)-2-oxo-2-[rac-(2R,5R)-4,4-difluoro-5-methyl-2-phenyl-1-piperidyl]acetamide

2-Oxo-2-[rac-(2R,5R)-4,4-difluoro-5-methyl-2-phenyl-1-piperidyl]acetamide (150.00 mg, 531.38 μmol), 4-bromo-2-tetrahydropyran-2-yl-pyrazolo[3,4-c]pyridin-7-amine (173.69 mg, 584.52 μmol), Copper (1.69 mg, 26.57 μmol), Copper (I) iodide (50.60 mg, 265.69 μmol, 9.00 μL), rac-(1R,2R)—N1,N2-dimethylcyclohexane-1,2-diamine (56.69 mg, 398.53 μmol) and Cesium carbonate (346.27 mg, 1.06 mmol) were mixed in Dioxane (4.99 mL). The reaction mixture was purged with argon for 5 min. Vials were sealed and heated at 100° C. for 15 hr. The reaction mixture was cooled and filtered. The filter cake was rinsed with MeOH (10 mL) and the filtrate was concentrated in vacuo to obtain N-(4-amino-2-tetrahydropyran-2-yl-pyrazolo[4,3-c]pyridin-7-yl)-2-oxo-2-[rac-(2R,5R)-4,4-difluoro-5-methyl-2-phenyl-1-piperidyl]acetamide (315 mg, crude) which was used in the next step without purification.

LCMS(ESI): [M+H]⁺ m/z: calcd 499.2; found 499.2; Rt=0.899 min.

### Step 4: The Synthesis of N-(4-Amino-1H-pyrazolo[4,3-c]pyridin-7-yl)-2-oxo-2-[rac-(2R,5R)-4,4-difluoro-5-methyl-2-phenyl-1-piperidyl]acetamide (Compound 216)

N-(4-Amino-2-tetrahydropyran-2-yl-pyrazolo[4,3-c]pyridin-7-yl)-2-oxo-2-[rac-(2R,5R)-4,4-difluoro-5-methyl-2-phenyl-1-piperidyl]acetamide (315 mg, 631.87 μmol) was dissolved in Methanol (3 mL) and Dioxane/HCl (3 mL). Resulting solution was stirred at rt for 15 hr. Solvents were evaporated. Resulting crude product was purified by HPLC (0-2-7 min, stop time=9 min 23-30-55% H₂O/MEOH/0.1NH₄OH; flow 30 mL/min ((loading pump 4 mL MeOH); column: XBridge BEH C18 100*19 mm, 5 microM) to obtain N-(4-amino-1H-pyrazolo[4,3-c]pyridin-7-yl)-2-oxo-2-[rac-(2R,5R)-4,4-difluoro-5-methyl-2-phenyl-1-piperidyl]acetamide (15.9 mg, 38.37 μmol, 6.07% yield).

¹H NMR (600 MHz, dmso) δ 1.01-1.15 (m, 3H), 2.12-2.30 (m, 1H), 2.83-3.04 (m, 2H), 3.47-3.78 (m, 2H), 3.97-4.44 (m, 1H), 5.32-6.32 (m, 1H), 6.60-7.02 (m, 2H), 7.05-7.57 (m, 5H), 7.60-8.18 (m, 1H), 9.46-10.91 (m, 1H), 12.39-13.51 (m, 1H).

LCMS(ESI): [M+H]⁺ m/z: calcd 415.2; found 415.2; Rt=1.006 min.

### Example 193. N-(4-Amino-1H-pyrazolo[4,3-c]pyridin-7-yl)-2-oxo-2-[rac-(2R,5S)-5-methyl-2-(2-thienyl)-1-piperidyl]acetamide (Compound 217)

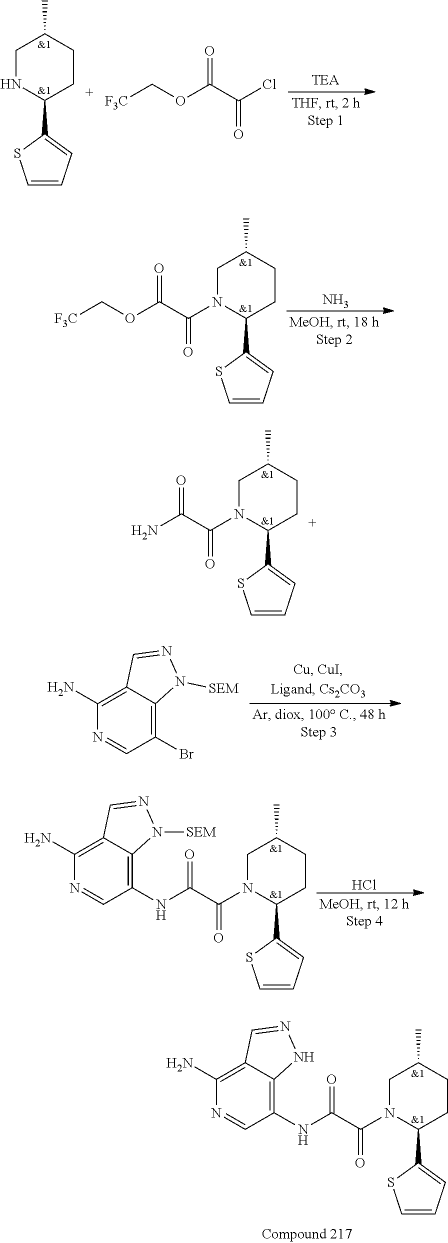

### Step 1: The Synthesis of 2,2,2-Trifluoroethyl 2-oxo-2-[rac-(2R,5S)-5-methyl-2-(2-thienyl)-1-piperidyl]acetate

To a solution of rac-(2R,5S)-5-methyl-2-(2-thienyl)piperidine (0.5 g, 2.76 mmol) and Triethylamine (418.61 mg, 4.14 mmol, 576.60 μL) in THF (30.19 mL) was added 2,2,2-trifluoroethyl 2-chloro-2-oxo-acetate (577.93 mg, 3.03 mmol) at rt. After stirring at rt for 1 hr the resulting mixture was washed with water, dried and evaporated to dryness to give 2,2,2-trifluoroethyl 2-oxo-2-[rac-(2R,5S)-5-methyl-2-(2-thienyl)-1-piperidyl]acetate (0.9 g, 2.68 mmol, 97.31% yield) as an yellow gum and was used in the next step without further purification.

LCMS(ESI): [M+H]⁺ m/z: calcd 336.4; found 336.4; Rt=3.754 min.

### Step 2: The Synthesis of 2-Oxo-2-[rac-(2R,5S)-5-methyl-2-(2-thienyl)-1-piperidyl]acetamide

Ammonia (45.71 mg, 2.68 mmol) was bubbled through a solution of 2,2,2-trifluoroethyl 2-oxo-2-[rac-(2R,5S)-5-methyl-2-(2-thienyl)-1-piperidyl]acetate (0.9 g, 2.68 mmol) in MeOH (25 mL) at rt. After stirring for 18 hr, the reaction mixture was evaporated to dryness and purified by CC (Interchim; 40 g SiO₂, chloroform/acetonitrile with acetonitrile from 0˜40%, flow rate=40 mL/min, Rv=9.5-12CV) to give 2-oxo-2-[rac-(2R,5S)-5-methyl-2-(2-thienyl)-1-piperidyl]acetamide (0.51 g, 2.02 mmol, 75.31% yield) as a light-yellow solid.

### Step 3: The Synthesis of N-[4-Amino-1-(2-trimethylsilylethoxymethyl)pyrazolo[4,3-c]pyridin-7-yl]-2-oxo-2-[rac-(2R,5S)-5-methyl-2-(2-thienyl)-1-piperidyl]acetamide

2-Oxo-2-[rac-(2R,5S)-5-methyl-2-(2-thienyl)-1-piperidyl]acetamide (50 mg, 198.15 μmol), 7-bromo-1-(2-trimethylsilylethoxymethyl)pyrazolo[4,3-c]pyridin-4-amine (68.02 mg, 198.15 μmol), Copper (I) iodide (18.87 mg, 99.08 μmol, 3.36 μL), Cesium carbonate (129.12 mg, 396.30 μmol) and (1R,2R)—N1,N2-dimethylcyclohexane-1,2-diamine (28.19 mg, 198.15 μmol) were mixed in dioxane (5.00 mL) under argon, and then stirred for 48 h at 100° C. for 48 hr in vial. The reaction mixture was filtered and concentrated in vacuo and purified by HPLC (40-65% 2-7 min; 30 mL/min water-acetonitrile+NH₃ (loading pump 4 mL/min acetonitrile); column XBridge 19*100 mm (L)). N-[4-Amino-1-(2-trimethylsilylethoxymethyl)pyrazolo[4,3-c]pyridin-7-yl]-2-oxo-2-[rac-(2R,5S)-5-methyl-2-(2-thienyl)-1-piperidyl]acetamide (30.5 mg, 59.26 μmol, 29.90% yield) was obtained as a beige solid.

LCMS(ESI): [M+H]⁺ m/z: calcd 515.2; found 515.2; Rt=3.543 min.

### Step 4: The Synthesis of N-(4-Amino-1H-pyrazolo[4,3-c]pyridin-7-yl)-2-oxo-2-[rac-(2R,5S)-5-methyl-2-(2-thienyl)-1-piperidyl]acetamide (Compound 217)

To a solution of N-[4-amino-1-(2-trimethylsilylethoxymethyl)pyrazolo[4,3-c]pyridin-7-yl]-2-oxo-2-[rac-(2R,5S)-5-methyl-2-(2-thienyl)-1-piperidyl]acetamide (30.5 mg, 59.26 μmol) in MeOH (1.75 mL) was added Hydrogen chloride solution 4.0M in dioxane (203.33 mg, 5.58 mmol, 254.17 μL) at 21° C. The resulting mixture was left to stir for 12 hr. The resulting mixture was evaporated to dryness and subjected to HPLC (1st run: 13-20-55% 0-2-7-7.1 min; 50 mL/min water-MeCN+NH₃ (loading pump 4 mL/min MeCN+NH₃); column Xbridge C18 5 uM 19*100 mm; 2nd run: 3-10-45-100% 0-2-9-9.1 min; 30 mL/min water-MeCN+FA (loading pump 4 mL/min; acetonitrile); column Sunfire C18 5 uM 19*100 mm). N-(4-amino-1H-pyrazolo[4,3-c]pyridin-7-yl)-2-oxo-2-[rac-(2R,5S)-5-methyl-2-(2-thienyl)-1-piperidyl]acetamide (6.1 mg, 14.17 μmol, 23.91% yield, HCOOH) was obtained as a colorless gum.

¹H NMR (600 MHz, dmso) δ 0.87-1.06 (m, 3H), 1.39-1.60 (m, 1H), 1.79-2.28 (m, 4H), 2.90-3.06 (m, 1H), 3.69-4.17 (m, 1H), 5.18-5.92 (m, 1H), 6.61-6.86 (m, 2H), 6.87-7.07 (m, 2H), 7.20-7.50 (m, 1H), 7.51-7.72 (m, 1H), 8.14-8.22 (m, 1H), 9.51-10.61 (m, 1H), 12.54-13.47 (m, 1H).

LCMS(ESI): [M+H]⁺ m/z: calcd 385.2; found 385.2; Rt=2.342 min.

### Example 194. N-(4-Amino-1H-pyrazolo[4,3-c]pyridin-7-yl)-2-oxo-2-[rac-(2R,5S)-5-methyl-2-(2-oxo-3,4-dihydro-1H-quinolin-6-yl)-1-piperidyl]acetamide (Compound 218)

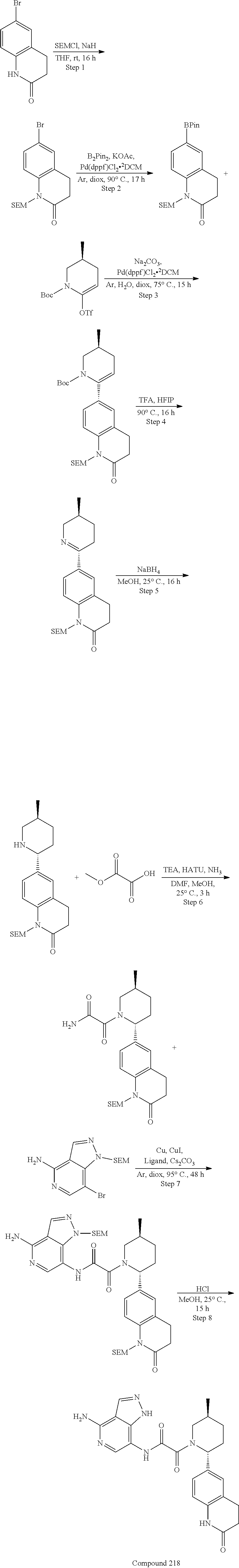

### Step 1: The Synthesis of 6-Bromo-1-(2-trimethylsilylethoxymethyl)-3,4-dihydroquinolin-2-one

Sodium hydride (in oil dispersion) 60% dispersion in mineral oil (1.12 g, 48.66 mmol) was added portion wise to a cooled to 0° C. solution of 6-bromo-3,4-dihydro-1H-quinolin-2-one (10 g, 44.23 mmol) in THF with stirring. The reaction mixture was stirred for 1 h and then 2-(chloromethoxy)ethyl-trimethyl-silane (8.85 g, 53.08 mmol, 9.39 mL) was added dropwise. The reaction mixture was stirred at RT overnight quenched with water and concentrated. The residue was portioned between water and DCM. The organic layer was dried over Na₂SO₄ and concentrated under reduced pressure to afford 6-bromo-1-(2-trimethylsilylethoxymethyl)-3,4-dihydroquinolin-2-one (15 g, crude) which was used in the next step without purification.

### Step 2: The Synthesis of 6-(4,4,5,5-Tetramethyl-1,3,2-dioxaborolan-2-yl)-1-(2-trimethylsilylethoxymethyl)-3,4-dihydroquinolin-2-one

6-Bromo-1-(2-trimethylsilylethoxymethyl)-3,4-dihydroquinolin-2-one (14.25 g, 39.99 mmol), 4,4,5,5-tetramethyl-2-(4,4,5,5-tetramethyl-1,3,2-dioxaborolan-2-yl)-1,3,2-dioxaborolane (10.86 g, 42.78 mmol), Potassium Acetate (15.70 g, 159.94 mmol, 10.00 mL) and Pd(dppf)Cl2 DCM (1.63 g, 2.00 mmol) were mixed in 1,4-dioxane (300 mL) and the reaction mixture was stirred under inert atmosphere at 90° C. for 17 hr. Upon completion, the reaction mixture was filtered through thin pad of SiO₂. The filtrate was diluted with DCM, washed with water and concentrated. The residue was purified by flash column chromatography to afford 6-(4,4,5,5-tetramethyl-1,3,2-dioxaborolan-2-yl)-1-(2-trimethylsilylethoxymethyl)-3,4-dihydroquinolin-2-one (4 g, 9.92 mmol, 24.80% yield). Chromatography data:

Interchim; 120 g SiO₂, HEX-MTBE from 0˜100%, flow rate=70 mL/min, cv=6.6

### Step 3: The Synthesis of tert-Butyl rac-(3S)-3-methyl-6-[2-oxo-1-(2-trimethylsilylethoxymethyl)-3,4-dihydroquinolin-6-yl]-3,4-dihydro-2H-pyridine-1-carboxylate

6-(4,4,5,5-Tetramethyl-1,3,2-dioxaborolan-2-yl)-1-(2-trimethylsilylethoxymethyl)-3,4-dihydroquinolin-2-one (4 g, 4.36 mmol), tert-butyl rac-(3S)-3-methyl-6-(trifluoromethylsulfonyloxy)-3,4-dihydro-2H-pyridine-1-carboxylate (2.26 g, 6.54 mmol), Sodium carbonate (1.39 g, 13.09 mmol, 547.91 μL) and Pd(dppf)Cl2 DCM (177.36 mg, 217.18 μmol) were stirred in a mixture of 1,4-dioxane (15 mL) and water (45 mL) under inert atmosphere at 75° C. for 15 hr. Upon completion, the reaction mixture was cooled down, diluted with water and extracted with DCM. The organic layer was separated, dried over Na₂SO₄ and concentrated under reduced pressure to afford tert-butyl rac-(3S)-3-methyl-6-[2-oxo-1-(2-trimethylsilylethoxymethyl)-3,4-dihydroquinolin-6-yl]-3,4-dihydro-2H-pyridine-1-carboxylate (2 g, crude) which was used in the next step without purification.

### Step 4: The Synthesis of 6-[rac-(3S)-3-Methyl-2,3,4,5-tetrahydropyridin-6-yl]-1-(2-trimethylsilylethoxymethyl)-3,4-dihydroquinolin-2-one

tert-Butyl rac-(3S)-3-methyl-6-[2-oxo-1-(2-trimethylsilylethoxymethyl)-3,4-dihydroquinolin-6-yl]-3,4-dihydro-2H-pyridine-1-carboxylate (2 g, 4.23 mmol) was dissolved in a mixture of TFA (740.00 mg, 6.49 mmol, 0.5 mL) and HFIP (710.98 mg, 4.23 mmol, 20 mL). The solution was stirred at 90° C. for 16 hr. Upon completion, the reaction mixture was concentrated under reduced pressure to afford 6-[rac-(3S)-3-methyl-2,3,4,5-tetrahydropyridin-6-yl]-1-(2-trimethylsilylethoxymethyl)-3,4-dihydroquinolin-2-one (2.1 g, crude, TFA) which was used in the next step without purification.

LCMS(ESI): [M+H]⁺ m/z: calcd 373.2; found 373.2; Rt=1.073 min.

### Step 5: The Synthesis of 6-[rac-(5S)-5-Methyl-2-piperidyl]-1-(2-trimethylsilylethoxymethyl)-3,4-dihydroquinolin-2-one

Sodium Borohydride (327.22 mg, 8.65 mmol, 304.68 μL) was added portionwise to a solution of 6-[rac-(3S)-3-methyl-2,3,4,5-tetrahydropyridin-6-yl]-1-(2-trimethylsilylethoxymethyl)-3,4-dihydroquinolin-2-one (2.1 g, 4.32 mmol, TFA) in methanol (20 mL) at RT. The reaction mixture was stirred overnight and concentrated under reduced pressure to afford 6-[rac-(5S)-5-methyl-2-piperidyl]-1-(2-trimethylsilylethoxymethyl)-3,4-dihydroquinolin-2-one (2 g, crude).

LCMS(ESI): [M+H]⁺ m/z: calcd 375.2; found 375.2; Rt=1.170 min.

### Step 6: The Synthesis of 2-Oxo-2-[rac-(2R,5S)-5-methyl-2-[2-oxo-1-(2-trimethylsilylethoxymethyl)-3,4-dihydroquinolin-6-yl]-1-piperidyl]acetamide

HATU (1.83 g, 4.81 mmol) was added in small portions at 25° C. to a stirred mixture of 6-[rac-(2R,5S)-5-methyl-2-piperidyl]-1-(2-trimethylsilylethoxymethyl)-3,4-dihydroquinolin-2-one (1.5 g, 4.00 mmol), 2-methoxy-2-oxo-acetic acid (625.05 mg, 6.01 mmol) and triethyl amine (2.03 g, 20.02 mmol, 2.79 mL) in DMF (4.46 mL). The resulting mixture was stirred at 25° C. for 2 hr. Then methanol (13.43 mL) was added and ammonia (68.19 mg, 4.00 mmol) was bubbled through reaction mixture during 20 min at 25° C. The formed precipitate was filtered off, washed with THF (50 mL) and the solvent was evaporated in vacuo to give crude product which was purified by reverse phase HPLC (SYSTEM 50-100% 0-5 min H₂O/MeOH/0.1% NH₄OH, flow: 30 mL/min (loading pump 4 mL/min methanol); column: XBridge C18 100×19 mm, 5 um) to give 2-oxo-2-[rac-(2R,5S)-5-methyl-2-[2-oxo-1-(2-trimethylsilylethoxymethyl)-3,4-dihydroquinolin-6-yl]-1-piperidyl]acetamide (0.2 g, 448.81 μmol, 11.21% yield).

LCMS(ESI): [M-SEM]⁺ m/z: calcd 329.2; found 329.2; Rt=1.421 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-5-methyl-2-[2-oxo-1-(2-trimethylsilylethoxymethyl)-3,4-dihydroquinolin-6-yl]-1-piperidyl]acetamide

A mixture of 2-oxo-2-[rac-(2R,5S)-5-methyl-2-[2-oxo-1-(2-trimethylsilylethoxymethyl)-3,4-dihydroquinolin-6-yl]-1-piperidyl]acetamide (200.00 mg, 448.81 μmol), 7-bromo-1-(2-trimethylsilylethoxymethyl)pyrazolo[4,3-c]pyridin-4-amine (154.07 mg, 448.81 μmol), Copper (I) iodide (85.48 mg, 448.81 μmol, 15.21 μL), Cesium carbonate (219.35 mg, 673.21 μmol), (1R,2R)—N1,N2-dimethylcyclohexane-1,2-diamine (95.76 mg, 673.21 μmol) and Cu (2.85 mg, 44.88 μmol) in Dioxane (3 mL) was stirred under argon at 95° C. for 48 hr. The reaction mixture was submitted for reverse phase HPLC (SYSTEM 60-100% 0-5 min H₂O/ACN/NH₄OH, flow: 30 mL/min (loading pump 4 mL/min acetonitrile); column: XBridge C18 OBD 100×19 mm 5 um) to give N-[4-amino-1-(2-trimethylsilylethoxymethyl)pyrazolo[4,3-c]pyridin-7-yl]-2-oxo-2-[rac-(2R,5S)-5-methyl-2-[2-oxo-1-(2-trimethylsilylethoxymethyl)-3,4-dihydroquinolin-6-yl]-1-piperidyl]acetamide (130 mg, 183.61 μmol, 40.91% yield).

LCMS(ESI): [M+H]⁺ m/z: calcd 708.4; found 708.4; Rt=1.352 min.

### Step 8: The Synthesis of N-(4-Amino-1H-pyrazolo[4,3-c]pyridin-7-yl)-2-oxo-2-[rac-(2R,5S)-5-methyl-2-(2-oxo-3,4-dihydro-1H-quinolin-6-yl)-1-piperidyl]acetamide (Compound 218)

To the stirred solution of N-[4-amino-1-(2-trimethylsilylethoxymethyl)pyrazolo[4,3-c]pyridin-7-yl]-2-oxo-2-[rac-(2R,5S)-5-methyl-2-[2-oxo-1-(2-trimethylsilylethoxymethyl)-3,4-dihydroquinolin-6-yl]-1-piperidyl]acetamide (130 mg, 183.61 μmol) in MeOH (1 mL) Hydrogen chloride solution 4.0M in dioxane (800.00 mg, 21.94 mmol, 1 mL) was added. The resulting mixture was stirred at 25° C. for 15 hr. The reaction mixture was concentrated under reduce pressure and was then purified by reverse phase HPLC (SYSTEM 0-0-55% 0-1-6 min H₂O/MeOH/0.1% FA, flow 30 mL/min (loading pump 4 mL/min MeOH); column: Kinetex PFP 100*21 2 mm 5 um) to give N-(4-amino-1H-pyrazolo[4,3-c]pyridin-7-yl)-2-oxo-2-[rac-(2R,5S)-5-methyl-2-(2-oxo-3,4-dihydro-1H-quinolin-6-yl)-1-piperidyl]acetamide (14 mg, 28.93 μmol, 15.76% yield, HCl).

¹H NMR (600 MHz, dmso) δ 0.91-1.07 (m, 3H), 1.18-1.40 (m, 1H), 1.46-2.25 (m, 4H), 2.40-2.44 (m, 2H), 2.63-2.90 (m, 3H), 3.70-4.08 (m, 1H), 4.87-5.59 (m, 1H), 6.55-6.89 (m, 3H), 6.94-7.23 (m, 2H), 7.50-7.77 (m, 1H), 8.15-9.62 (m, 1H), 9.86-10.52 (m, 2H), 12.62-13.35 (m, 1H).

LCMS(ESI): [M+H]⁺ m/z: calcd 448.2; found 448.2; Rt=2.165 min.

### Example 195. N-(4-Amino-1H-pyrazolo[4,3-c]pyridin-7-yl)-2-oxo-2-[(2R,5S)-2-(2-methoxy-6-methyl-4-pyridyl)-5-methyl-1-piperidyl]acetamide (Compound 219)

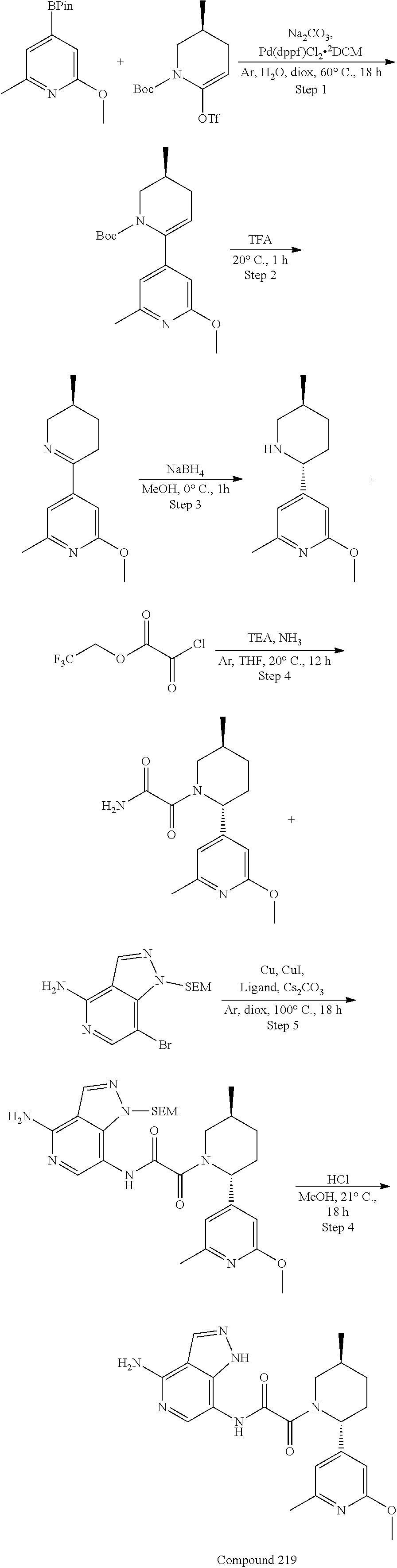

### Step 1: The Synthesis of tert-Butyl (3S)-6-(2-methoxy-6-methyl-4-pyridyl)-3-methyl-3,4-dihydro-2H-pyridine-1-carboxylate

A mixture of 2-methoxy-6-methyl-4-(4,4,5,5-tetramethyl-1,3,2-dioxaborolan-2-yl)pyridine (6.3 g, 25.29 mmol), tert-butyl (3S)-3-methyl-6-(trifluoromethylsulfonyloxy)-3,4-dihydro-2H-pyridine-1-carboxylate (8.73 g, 25.29 mmol), cyclopentyl(diphenyl)phosphane; dichloromethane; dichloropalladium; iron (1.03 g, 1.26 mmol) and sodium carbonate (8.04 g, 75.87 mmol, 3.18 mL) in dioxane (90 mL) and water (30 mL) was stirred at 60° C. under argon atmosphere for 18 hr. The reaction mixture was filtered off and the filtrate was evaporated in vacuo. The residue was purified by silica gel flash chromatography eluting with a 0 to 100 percent DCM-EA gradient to afford product tert-butyl (3S)-6-(2-methoxy-6-methyl-4-pyridyl)-3-methyl-3,4-dihydro-2H-pyridine-1-carboxylate (8 g, 25.12 mmol, 99.35% yield).

LCMS(ESI): [M+H]⁺ m/z: calcd 319.2; found 319.2; Rt=1.340 min.

### Step 2: The Synthesis of 2-Methoxy-6-methyl-4-[rac-(3S)-3-methyl-2,3,4,5-tetrahydropyridin-6-yl]pyridine

The solution of tert-butyl rac-(3S)-6-(2-methoxy-6-methyl-4-pyridyl)-3-methyl-3,4-dihydro-2H-pyridine-1-carboxylate (8 g, 25.12 mmol) in TFA (45.84 g, 402.00 mmol, 30.97 mL) was stirred at 20° C. for 1 hr, and then evaporated in vacuo. Crushed ice (50 g) was added to the residue and the pH was adjusted to 10 with a 10% aqueous solution of potassium carbonate. The resulting mixture was extracted with EA (2*75 mL). The combined organic extracts were dried over sodium sulphate and evaporated in vacuo to afford 2-methoxy-6-methyl-4-[rac-(3S)-3-methyl-2,3,4,5-tetrahydropyridin-6-yl]pyridine (5.2 g, 23.82 mmol, 94.81% yield) as yellow solid, which was used directly in the next step.

LCMS(ESI): [M+H]⁺ m/z: calcd 219.2; found 219.2; Rt=0.693 min.

### Step 3: The Synthesis of 2-Methoxy-6-methyl-4-[rac-(2R,5S)-5-methyl-2-piperidyl]pyridine

Sodium borohydride (1.35 g, 35.73 mmol, 1.26 mL) was added in one portion to a stirred solution of 2-methoxy-6-methyl-4-[rac-(3S)-3-methyl-2,3,4,5-tetrahydropyridin-6-yl]pyridine (5.2 g, 23.82 mmol) in methanol (100 mL) at 0° C. The resulting mixture was stirred at 0° C. for 1 hr, and then evaporated in vacuo. The residue was diluted with water (100 mL) and extracted with dichloromethane (2*75 mL). The combined organic extracts were dried over sodium sulphate and evaporated in vacuo to afford 2-methoxy-6-methyl-4-[rac-(2R,5S)-5-methyl-2-piperidyl]pyridine (3.78 g, 17.16 mmol, 72.03% yield) as yellow solid, which was used directly in the next step.

LCMS(ESI): [M+H]⁺ m/z: calcd 221.1; found 221.1; Rt=0.805 min.

### Step 4: The Synthesis of 2-Oxo-2-[(2R,5S)-2-(2-methoxy-6-methyl-4-pyridyl)-5-methyl-1-piperidyl]acetamide

To a solution of 2-methoxy-6-methyl-4-[(2R,5S)-5-methyl-2-piperidyl]pyridine (3.78 g, 17.16 mmol) and TEA (3.47 g, 34.32 mmol, 4.78 mL) in THF (50 mL) was added 2,2,2-trifluoroethyl 2-chloro-2-oxo-acetate (4.90 g, 25.74 mmol) dropwise at 0° C. under argon. The reaction mixture was then stirred for 12 hr at r.t. Then ammonia (292.20 mg, 17.16 mmol) was bubbled through for 10 min at 0° C. The reaction mixture was then stirred for 12 hr at r.t. The reaction mixture was filtered off and the filtrate was evaporated in vacuo to give 2-oxo-2-[(2R,5S)-2-(2-methoxy-6-methyl-4-pyridyl)-5-methyl-1-piperidyl]acetamide (3 g, 10.30 mmol, 60.01% yield).

LCMS(ESI): [M+H]⁺ m/z: calcd 292.2; found 292.2; Rt=0.791 min.

### Step 5: The Synthesis of N-[4-Amino-1-(2-trimethylsilylethoxymethyl)pyrazolo[4,3-c]pyridin-7-yl]-2-oxo-2-[(2R,5S)-2-(2-methoxy-6-methyl-4-pyridyl)-5-methyl-1-piperidyl]acetamide

7-Bromo-1-(2-trimethylsilylethoxymethyl)pyrazolo[4,3-c]pyridin-4-amine (380 mg, 1.11 mmol), 2-oxo-2-[(2R,5S)-2-(2-methoxy-6-methyl-4-pyridyl)-5-methyl-1-piperidyl]acetamide (322.50 mg, 1.11 mmol), Cu (7.46 mg, 117.33 μmol), CuI (63.24 mg, 332.08 μmol, 11.25 μL), Caesium carbonate (540.99 mg, 1.66 mmol, 236.24 μL) and (1R,2R)—N1,N2-dimethylcyclohexane-1,2-diamine (47.23 mg, 332.08 μmol) were mixed in dioxane (5 mL), purged with Ar for 5 minutes and then heated in the sealed tube at 100° C. for 18 hr. Final mixture was filtered and dioxane was evaporated in vacuo. The residue was purified by RP-HPLC (column: XBridge BEH C18 5 um 130 A; 50-50-95% 0-1-6 min H₂O/MeOH/0.1% NH₄OH, flow: 30 mL/min, flow: 30 mL/min) to afford N-[4-amino-1-(2-trimethylsilylethoxymethyl)pyrazolo[4,3-c]pyridin-7-yl]-2-oxo-2-[(2R,5S)-2-(2-methoxy-6-methyl-4-pyridyl)-5-methyl-1-piperidyl]acetamide (200 mg, 361.19 μmol, 32.63% yield) as light-brown solid (mixture of cis/trans), which was used directly in the next step.

LCMS(ESI): [M+H]⁺ m/z: calcd 556.2; found 556.2; Rt=1.290 min.

### Step 6: The Synthesis of N-(4-Amino-1H-pyrazolo[4,3-c]pyridin-7-yl)-2-oxo-2-[(2R,5S)-2-(2-methoxy-6-methyl-4-pyridyl)-5-methyl-1-piperidyl]acetamide (Compound 219)

To a solution of N-[4-amino-1-(2-trimethylsilylethoxymethyl)pyrazolo[4,3-c]pyridin-7-yl]-2-oxo-2-[(2R,5S)-2-(2-methoxy-6-methyl-4-pyridyl)-5-methyl-1-piperidyl]acetamide (160.71 mg, 290.24 μmol) in methanol (3 mL) was added Hydrogen chloride solution 4.0M in dioxane (1.59 g, 4.35 mmol, 1.98 mL, 10% purity) at 21° C. The resulting mixture was left to stir for 18 hr. The resulting mixture was evaporated to dryness. The residue was purified by RP-HPLC (column: YMC Triart C18 100×20 mm, 5 um; 30-60% 0-5 min H₂O/MeOH/0.1% NH₄OH, flow: 30 mL/min) to give Compound 219 N-(4-amino-1H-pyrazolo[4,3-c]pyridin-7-yl)-2-oxo-2-[(2R,5S)-2-(2-methoxy-6-methyl-4-pyridyl)-5-methyl-1-piperidyl]acetamide (25.9 mg, 61.16 μmol, 21.07% yield).

¹H NMR (600 MHz, dmso) δ 0.84-1.06 (m, 3H), 1.20-1.41 (m, 1H), 1.52-1.68 (m, 1H), 1.69-2.22 (m, 3H), 2.31-2.41 (m, 3H), 2.72-3.26 (m, 1H), 3.73-4.19 (m, 4H), 4.84-5.96 (m, 1H), 6.23-6.92 (m, 4H), 7.44-7.76 (m, 1H), 8.07-8.28 (m, 1H), 9.52-10.61 (m, 1H), 12.54-13.37 (m, 1H).

LCMS(ESI): [M+H]⁺ m/z: calcd 424.2; found 424.2; Rt=1.875 min.

### Example 196. N-(4-Amino-2H-pyrazolo[4,3-c]pyridin-7-yl)-2-oxo-2-[rac-(2R,5S)-2-(4,4-difluorocyclohexyl)-5-methyl-1-piperidyl]acetamide (Compound 220)

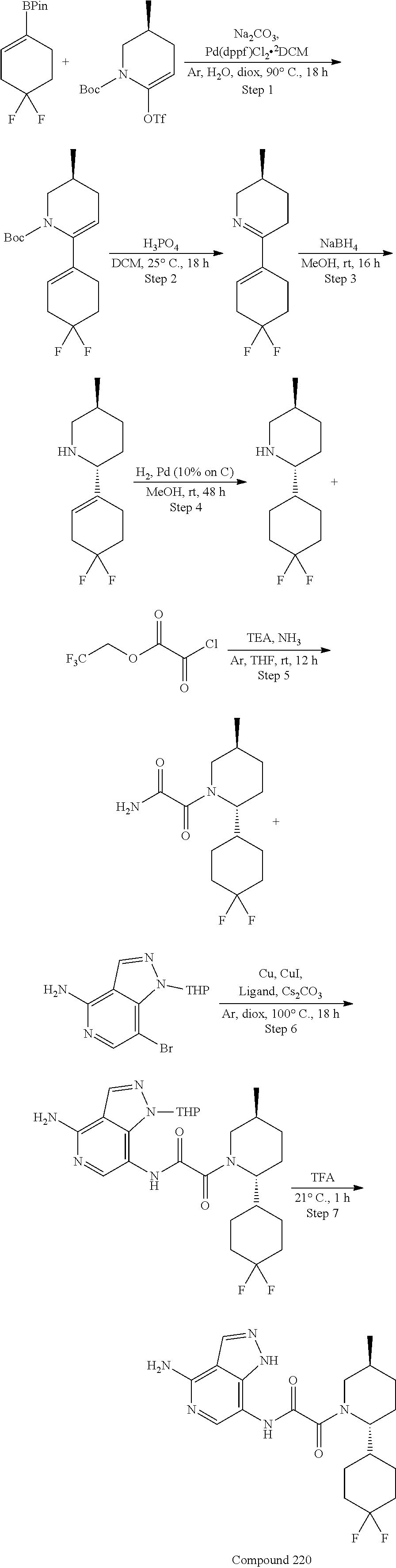

### Step 1: The Synthesis of tert-Butyl rac-(3S)-6-(4,4-difluorocyclohexen-1-yl)-3-methyl-3,4-dihydro-2H-pyridine-1-carboxylate

A mixture of 2-(4,4-difluorocyclohexen-1-yl)-4,4,5,5-tetramethyl-1,3,2-dioxaborolane (4 g, 16.39 mmol), tert-butyl (3S)-3-methyl-6-(trifluoromethylsulfonyloxy)-3,4-dihydro-2H-pyridine-1-carboxylate (5.66 g, 16.39 mmol), cyclopentyl(diphenyl)phosphane; dichloromethane; dichloropalladium; iron (669.14 mg, 819.39 μmol) and sodium carbonate (5.21 g, 49.16 mmol, 2.06 mL) in dioxane (182.83 mL) and water (60.94 mL) was stirred at 90° C. under argon atmosphere for 18 hr. The reaction mixture was filtered off and the filtrate was evaporated in vacuo. The residue was diluted with water (100 mL) and extracted with MTBE (2*75 mL). The combined organic extracts were dried over sodium sulphate and evaporated in vacuo. The residue was purified by silica gel flash chromatography eluting with a 0 to 100 percent hexane-MTBE gradient to afford product tert-butyl rac-(3S)-6-(4,4-difluorocyclohexen-1-yl)-3-methyl-3,4-dihydro-2H-pyridine-1-carboxylate (2 g, 6.38 mmol, 38.94% yield).

LCMS(ESI): [M−tBu]⁺ m/z: calcd 258.2; found 258.2; Rt=1.522 min.

### Step 2: The Synthesis of rac-(3S)-6-(4,4-Difluorocyclohexen-1-yl)-3-methyl-2,3,4,5-tetrahydropyridine

Phosphoric acid, 85% aq. soln. (3.75 g, 38.29 mmol, 2.21 mL) was added to a stirred solution of tert-butyl rac-(3S)-6-(4,4-difluorocyclohexen-1-yl)-3-methyl-3,4-dihydro-2H-pyridine-1-carboxylate (1.20 g, 3.83 mmol) in dichloromethane (25 mL). The resulting mixture was stirred at 25° C. for 18 hr, then upper dichloromethane layer was decanted. Crushed ice (10 g) and water (20 ml) ware added to orange residue, which left after decantation of dichloromethene layer and resulting mixture was carefully basified to pH 10 with 10% aqueous solution of sodium hydroxide. The light-yellow cloudy solution was extracted with dichloromethene (2*50 ml). The combined organic extracts were dried over sodium sulphate and carefully evaporated in vacuo (bath temp. 15-20° C.) to leave rac-(3S)-6-(4,4-difluorocyclohexen-1-yl)-3-methyl-2,3,4,5-tetrahydropyridine (0.5 g, crude) as light-yellow gum, which was immediately used in the next step due to a unstable nature of the product.

LCMS(ESI): [M+H]⁺ m/z: calcd 214.2; found 214.2; Rt=0.712 min.

### Step 3: The Synthesis of rac-(2R,5S)-2-(4,4-Difluorocyclohexen-1-yl)-5-methyl-piperidine

Sodium Borohydride (133.04 mg, 3.52 mmol, 123.87 μL) was added in one portion to a stirred solution of rac-(3S)-6-(4,4-difluorocyclohexen-1-yl)-3-methyl-2,3,4,5-tetrahydropyridine (500.00 mg, 2.34 mmol) in methanol (20.02 mL) at 0° C. The resulting mixture was stirred at 0° C. for 1 hr, and then evaporated in vacuo. The residue was diluted with water (10 mL) and extracted with dichloromethane (2*30 mL). The combined organic extracts were dried over sodium sulphate and evaporated in vacuo to afford rac-(2R,5S)-2-(4,4-difluorocyclohexen-1-yl)-5-methyl-piperidine (0.5 g, crude) as yellow oil, which was used directly in the next step.

LCMS(ESI): [M+H]⁺ m/z: calcd 216.2; found 216.2; Rt=0.825 min.

### Step 4: The Synthesis of rac-(2R,5S)-2-(4,4-Difluorocyclohexyl)-5-methyl-piperidine

A mixture of rac-(2R,5S)-2-(4,4-difluorocyclohexen-1-yl)-5-methyl-piperidine (0.5 g, 2.32 mmol) and Palladium, 10% on carbon (0.5 g, 2.32 mmol) in methanol (30 mL) was stirred under atmosphere of hydrogen at 25° C. for 48 hr. The catalyst was filtered off, the filtrate was evaporated in vacuo to afford rac-(2R,5S)-2-(4,4-difluorocyclohexyl)-5-methyl-piperidine (0.5 g, crude) as yellow oil, which was used directly in the next step.

LCMS(ESI): [M+H]⁺ m/z: calcd 218.2; found 218.2; Rt=0.709 min.

### Step 5: The Synthesis of 2-Oxo-2-[rac-(2R,5S)-2-(4,4-difluorocyclohexyl)-5-methyl-1-piperidyl]acetamide

To a solution of rac-(2R,5S)-2-(4,4-difluorocyclohexyl)-5-methyl-piperidine (500.00 mg, 2.30 mmol) and TEA (465.67 mg, 4.60 mmol, 641.42 μL) in THF (10.21 mL) was added 2,2,2-trifluoroethyl 2-chloro-2-oxo-acetate (657.53 mg, 3.45 mmol) dropwise at 0° C. under argon. The reaction mixture was then stirred for 12 hr at r.t. Then ammonia (292.20 mg, 17.16 mmol) was bubbled through for 10 min at 0° C. The reaction mixture was then stirred for 12 hr at r.t. The reaction mixture was filtered off and the filtrate was evaporated in vacuo to give 2-oxo-2-[rac-(2R,5S)-2-(4,4-difluorocyclohexyl)-5-methyl-1-piperidyl]acetamide (0.6 g, crude).

LCMS(ESI): [M+H]⁺ m/z: calcd 289.2; found 289.2; Rt=0.981 min.

### Step 6: The Synthesis of N-(4-Amino-2-tetrahydropyran-2-yl-pyrazolo[4,3-c]pyridin-7-yl)-2-oxo-2-[rac-(2R,5S)-2-(4,4-difluorocyclohexyl)-5-methyl-1-piperidyl]acetamide

2-Oxo-2-[rac-(2R,5S)-2-(4,4-difluorocyclohexyl)-5-methyl-1-piperidyl]acetamide (250.00 mg, 867.05 μmol), 7-bromo-2-tetrahydropyran-2-yl-pyrazolo[4,3-c]pyridin-4-amine (386.47 mg, 1.30 mmol), Cu (5.84 mg, 91.91 μmol), CuI (49.54 mg, 260.12 μmol, 8.81 μL), Caesium carbonate (423.76 mg, 1.30 mmol, 185.05 μL) and (1R,2R)—N1,N2-dimethylcyclohexane-1,2-diamine (37.00 mg, 260.12 μmol) were mixed in dioxane (8.26 mL), purged with Ar for 2 minutes and then heated in the sealed tube at 100° C. for 18 hr. Final mixture was filtered and the filtrate was evaporated in vacuo to afford N-(4-amino-2-tetrahydropyran-2-yl-pyrazolo[4,3-c]pyridin-7-yl)-2-oxo-2-[rac-(2R,5S)-2-(4,4-difluorocyclohexyl)-5-methyl-1-piperidyl]acetamide (0.5 g, crude) as brown solid, which was used directly in the next step.

LCMS(ESI): [M+H]⁺ m/z: calcd 505.2; found 505.2; Rt=1.001 min.

### Step 7: The Synthesis of N-(4-Amino-2H-pyrazolo[4,3-c]pyridin-7-yl)-2-oxo-2-[rac-(2R,5S)-2-(4,4-difluorocyclohexyl)-5-methyl-1-piperidyl]acetamide (Compound 220)

To N-(4-amino-2-tetrahydropyran-2-yl-pyrazolo[4,3-c]pyridin-7-yl)-2-oxo-2-[rac-(2R,5S)-2-(4,4-difluorocyclohexyl)-5-methyl-1-piperidyl]acetamide (700.00 mg, 1.39 mmol) was added TFA (3.16 g, 27.75 mmol, 2.12 mL) at 21° C. The resulting mixture was left to stir for 1 hr. The resulting mixture was evaporated to dryness. The residue was diluted with NH₄OH (2 mL 10% aq.) and purified by RP-HPLC (column: XBridge C18 OBD 100×30 mm 5 um; 25-25-55% 0-1-5 min H₂O/ACN/NH₄OH flow: 60 mL/min) to give Compound 220 N-(4-amino-2H-pyrazolo[4,3-c]pyridin-7-yl)-2-oxo-2-[rac-(2R,5S)-2-(4,4-difluorocyclohexyl)-5-methyl-1-piperidyl]acetamide (50.0 mg, 8.57%).

LCMS(ESI): [M+H]⁺ m/z: calcd 421.2; found 421.2; Rt=2.200 min.

### Example 197. N-(4-Amino-2H-pyrazolo[4,3-c]pyridin-7-yl)-2-oxo-2-[rac-(2R,5S)-5-methyl-2-(1,1,2-trimethyl-3,4-dihydroisoquinolin-6-yl)-1-piperidyl]acetamide (Compound 221)

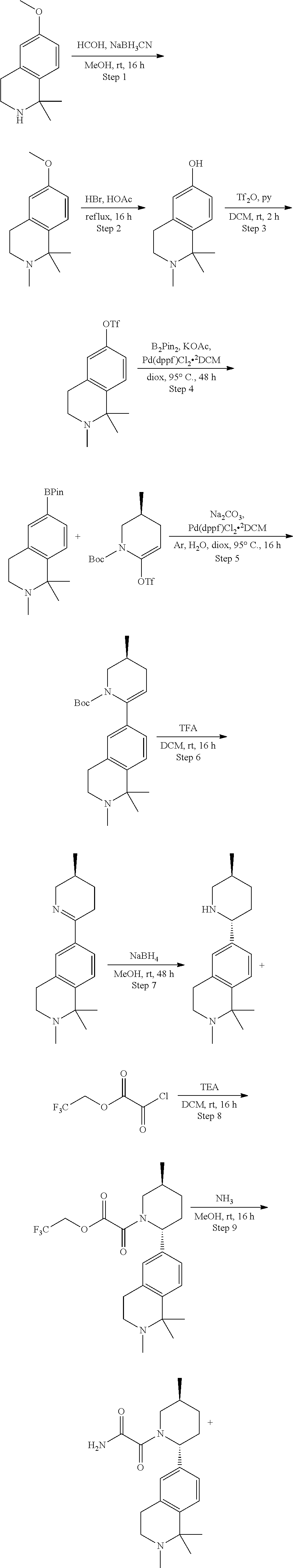

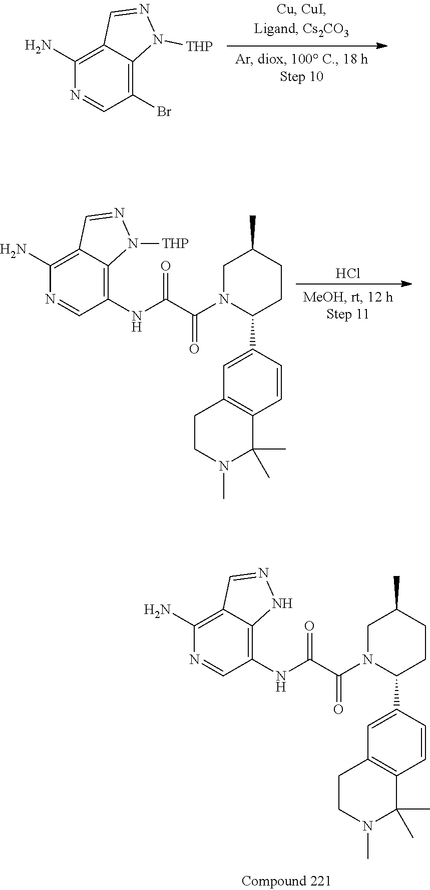

### Step 1: The Synthesis of 6-Methoxy-1,1,2-trimethyl-3,4-dihydroisoquinoline

6-Methoxy-1,1-dimethyl-3,4-dihydro-2H-isoquinoline (3.17 g, 13.92 mmol, HCl) was dissolved in 40 ml water, K₂CO₃ was added to alkaline pH and the resulting mixture was extracted with DCM (3*20 mL), combined organic layers were dried and evaporated to obtain 6-methoxy-1,1-dimethyl-3,4-dihydro-2H-isoquinoline (3.17 g, 13.92 mmol, HCl) in basic form. Formaldehyde, 37% in aq. soln., ACS, 36.5-38.0%, stab. with 10-15% methanol (1.46 g, 48.72 mmol, 1.35 mL) and acetic acid (1.67 g, 27.84 mmol, 1.59 mL) were added to a stirred solution of basic form of 6-methoxy-1,1-dimethyl-3,4-dihydro-2H-isoquinoline (3.17 g, 13.92 mmol, HCl) in MeOH (58.17 mL) at 25° C. The resulting mixture was stirred at 25° C. for 0.5 hr, then Sodium cyanoborohydride (1.75 g, 27.84 mmol) was added in one portion at 25° C. (foaming!). The reaction mixture was stirred at 25° C. for 15 hr, and then concentrated in vacuo. The residue was diluted with 10% aqueous sodium hydroxide solution (40 mL) and extracted with dichloromethane (2*20 mL). The combined organic extracts were dried over sodium sulphate and concentrated in vacuo to afford 6-methoxy-1,1,2-trimethyl-3,4-dihydroisoquinoline (2 g, 9.74 mmol, 69.99% yield).

LCMS(ESI): [M+H]⁺ m/z: calcd 206.2; found 206.2; Rt=0.579 min.

### Step 2: The Synthesis of 1,1,2-Trimethyl-3,4-dihydroisoquinolin-6-ol

To a mixture of Hydrobromic acid, 48% (25.33 g, 313.06 mmol, 17 mL) and acetic acid (2.62 g, 43.67 mmol, 2.5 mL), 6-methoxy-1,1,2-trimethyl-3,4-dihydroisoquinoline (1.7 g, 8.28 mmol) was added and the resulting mixture was heated at reflux overnight. Upon completion of the reaction mixture was evaporated in vacuo, 20 ml water was added, the resulting mixture was basified with K₂CO₃ to alkaline pH and extracted with DCM (3*20 ml) combined organic layers were dried over Na₂SO₄, filtered and concentrated in vacuo to obtain 1,1,2-trimethyl-3,4-dihydroisoquinolin-6-ol (1.2 g, 6.27 mmol, 75.76% yield).

LCMS(ESI): [M+H]⁺ m/z: calcd 192.2; found 192.2; Rt=0.598 min.

### Step 3: The Synthesis of (1,1,2-Trimethyl-3,4-dihydroisoquinolin-6-yl) trifluoromethanesulfonate

To a mixture of 1,1,2-trimethyl-3,4-dihydroisoquinolin-6-ol (1.4 g, 7.32 mmol) in DCM (50 mL), pyridine (1.16 g, 14.64 mmol, 1.18 mL) was added in one portion and then the resulting mixture was cooled to 0° C. and Trifluoromethanesulfonic anhydride (2.48 g, 8.78 mmol, 1.48 mL) was added dropwise, mixture was heated up to room temperature for 2 hr. After that time, the resulting mixture was washed with brine (3*15 mL), organic layer was dried over Na₂SO₄, filtered and concentrated in vacuo to afford (1,1,2-trimethyl-3,4-dihydroisoquinolin-6-yl) trifluoromethanesulfonate (2.3 g, crude).

LCMS(ESI): [M+2H]⁺ m/z: calcd 325.2; found 325.2; Rt=0.755 min.

### Step 4: The Synthesis of 1,1,2-Trimethyl-6-(4,4,5,5-tetramethyl-1,3,2-dioxaborolan-2-yl)-3,4-dihydroisoquinoline

A mixture of (1,1,2-trimethyl-3,4-dihydroisoquinolin-6-yl) trifluoromethanesulfonate (2.3 g, 7.11 mmol), 4,4,5,5-tetramethyl-2-(4,4,5,5-tetramethyl-1,3,2-dioxaborolan-2-yl)-1,3,2-dioxaborolane (1.81 g, 7.11 mmol) and Potassium Acetate (1.40 g, 14.23 mmol, 889.34 μL) in Dioxane (50 mL) was evacuated and then backfilled with argon. This operation was repeated two times, then Pd(dppf)Cl₂·CH₂Cl₂ (290.46 mg, 355.67 μmol) was added and the reaction mixture was stirred under argon at 95° C. for 48 hr. Upon completion, the mixture was cooled to r.t. and poured into 60 mL water, the resulting mixture was extracted with DCM (3*20 mL), dried over Na₂SO₄, filtered and concentrated in vacuo to afford 1,1,2-trimethyl-6-(4,4,5,5-tetramethyl-1,3,2-dioxaborolan-2-yl)-3,4-dihydroisoquinoline (2.4 g, crude).

LCMS(ESI): [M+H]⁺ m/z: calcd 302.2; found 302.2; Rt=0.876 min.

### Step 5: The Synthesis of tert-Butyl rac-(3S)-3-methyl-6-(1,1,2-trimethyl-3,4-dihydroisoquinolin-6-yl)-3,4-dihydro-2H-pyridine-1-carboxylate

1,1,2-Trimethyl-6-(4,4,5,5-tetramethyl-1,3,2-dioxaborolan-2-yl)-3,4-dihydroisoquinoline (2.3 g, 7.64 mmol) and tert-butyl rac-(3S)-3-methyl-6-(trifluoromethylsulfonyloxy)-3,4-dihydro-2H-pyridine-1-carboxylate (3.69 g, 10.69 mmol) were mixed together in the Dioxane (39.80 mL) and Sodium carbonate (1.62 g, 15.27 mmol, 639.23 μL) (solution in the Water (19.90 mL)) was added thereto. The resulting mixture was evacuated and backfilled with argon, this operation was repeated three times. Cyclopentyl(diphenyl)phosphane; dichloromethane; dichloropalladium; iron (311.77 mg, 381.77 μmol) was added to the reaction mixture under argon stream and this reaction mixture was stirred at 95° C. for 16 hr. After completion the reaction mixture was cooled to the room temperature and solvents were evaporated under reduced pressure. To the resulting mixture 40 mL water was added and after 10 min stirred mixture was extracted with DCM (3*20 mL), combined organic layers were dried over Na₂SO₄, filtered and concentrated in vacuo to afford tert-butyl rac-(3S)-3-methyl-6-(1,1,2-trimethyl-3,4-dihydroisoquinolin-6-yl)-3,4-dihydro-2H-pyridine-1-carboxylate (3.7 g, crude).

LCMS(ESI): [M+H]⁺ m/z: calcd 371.2; found 371.2; Rt=1.230 min.

### Step 6: The Synthesis of 1,1,2-Trimethyl-6-[rac-(3S)-3-methyl-2,3,4,5-tetrahydropyridin-6-yl]-3,4-dihydroisoquinoline

tert-Butyl rac-(3S)-3-methyl-6-(1,1,2-trimethyl-3,4-dihydroisoquinolin-6-yl)-3,4-dihydro-2H-pyridine-1-carboxylate (3.7 g, 9.99 mmol) was dissolved in DCM (80 mL), after stirred for 10 min, TFA (11.39 g, 99.86 mmol, 7.69 mL) was added portionwise and the resulting mixture was left to stir at 25° C. for 16 hr. After completion the solvent was evaporated under reduced pressure and the residue was diluted with warm water (40 mL) and this solution was decanted from the black resin. K₂CO₃ was added to the aqueous solution to alkaline, extracted with DCM (3*20 mL), combined organic layers were dried over Na₂SO₄, filtered and concentrated in vacuo to afford 1,1,2-trimethyl-6-[rac-(3S)-3-methyl-2,3,4,5-tetrahydropyridin-6-yl]-3,4-dihydroisoquinoline (1.25 g, crude).

LCMS(ESI): [M+H]⁺ m/z: calcd 271.2; found 271.2; Rt=0.500 min.

### Step 7: The Synthesis of 1,1,2-Trimethyl-6-[rac-(5S)-5-methyl-2-piperidyl]-3,4-dihydroisoquinoline

1,1,2-Trimethyl-6-[rac-(3S)-3-methyl-2,3,4,5-tetrahydropyridin-6-yl]-3,4-dihydroisoquinoline (1.2 g, 4.44 mmol) was dissolved in MeOH (50 mL). After 10 min, Sodium Borohydride (251.83 mg, 6.66 mmol, 234.48 μL) was added in one portion and the resulting mixture was stirred at 25° C. for 48 hr. Upon completion of the reaction, solvent was evaporated to dryness, 40 mL water was added, and the resulting mixture was extracted with DCM (3*20 mL), combined organic layers were dried over Na₂SO₄, filtered and evaporated under reduced pressure to obtain 1,1,2-trimethyl-6-[rac-(5S)-5-methyl-2-piperidyl]-3,4-dihydroisoquinoline (0.9 g, 3.30 mmol, 74.45% yield).

LCMS(ESI): [M+H]⁺ m/z: calcd 273.2; found 273.2; Rt=0.588 min.

### Step 8: The Synthesis of 2,2,2-Trifluoroethyl 2-oxo-2-[rac-(5S)-5-methyl-2-(1,1,2-trimethyl-3,4-dihydroisoquinolin-6-yl)-1-piperidyl]acetate

1,1,2-Trimethyl-6-[rac-(5S)-5-methyl-2-piperidyl]-3,4-dihydroisoquinoline (0.9 g, 3.30 mmol) and TEA (668.59 mg, 6.61 mmol, 920.92 μL) were mixed together in DCM (30 mL) and the resulting solution was cooled to 5° C. in an ice bath. 2,2,2-Trifluoroethyl 2-chloro-2-oxo-acetate (944.04 mg, 4.96 mmol) was added dropwise to the previous solution and the resulting mixture was allowed to warm to rt and stirred for 16 hr. Upon completion of the reaction, the mixture was washed with brine (2*20 mL), organic layer was dried over Na₂SO₄, filtered and concentrated in vacuo to affording crude 2,2,2-trifluoroethyl 2-oxo-2-[rac-(5S)-5-methyl-2-(1,1,2-trimethyl-3,4-dihydroisoquinolin-6-yl)-1-piperidyl]acetate (1.4 g, crude).

LCMS(ESI): [M+H]⁺ m/z: calcd 427.0; found 427.0; Rt=1.041 min.

### Step 9: The Synthesis of 2-Oxo-2-[rac-(5S)-5-methyl-2-(1,1,2-trimethyl-3,4-dihydroisoquinolin-6-yl)-1-piperidyl]acetamide

2,2,2-Trifluoroethyl 2-oxo-2-[rac-(5S)-5-methyl-2-(1,1,2-trimethyl-3,4-dihydroisoquinolin-6-yl)-1-piperidyl]acetate (1.4 g, 3.28 mmol) was dissolved in NH₃/MeOH (30 mL) and the resulting mixture was stirred at 25° C. for 16 hr. Upon completion of the reaction mixture was evaporated to dryness to obtain 2-oxo-2-[rac-(5S)-5-methyl-2-(1,1,2-trimethyl-3,4-dihydroisoquinolin-6-yl)-1-piperidyl]acetamide (1 g, crude).

### Step 10: The Synthesis of N-(4-Amino-2-tetrahydropyran-2-yl-pyrazolo[4,3-c]pyridin-7-yl)-2-oxo-2-[rac-(2R,5S)-5-methyl-2-(1,1,2-trimethyl-3,4-dihydroisoquinolin-6-yl)-1-piperidyl]acetamide

To a mixture of 7-bromo-2-tetrahydropyran-2-yl-pyrazolo[4,3-c]pyridin-4-amine (389.32 mg, 1.31 mmol), 2-oxo-2-[rac-(2R,5S)-5-methyl-2-(1,1,2-trimethyl-3,4-dihydroisoquinolin-6-yl)-1-piperidyl]acetamide (0.3 g, 873.46 μmol), Copper (11.10 mg, 174.69 μmol), Copper (I) iodide (166.35 mg, 873.46 μmol, 29.60 μL), Cesium carbonate (569.18 mg, 1.75 mmol) and rac-(1R,2R)—N1,N2-dimethylcyclohexane-1,2-diamine (186.36 mg, 1.31 mmol), Dioxane (5.00 mL) was added. The resulting mixture was evacuated, refiled with Argon three time, heated at 100° C. for 18 hr and cooled. The precipitate was filtered through a thin layer of SiO₂ and washed with DCM (30 mL). The solvent was evaporated to give N-(4-amino-2-tetrahydropyran-2-yl-pyrazolo[4,3-c]pyridin-7-yl)-2-oxo-2-[rac-(2R,5S)-5-methyl-2-(1,1,2-trimethyl-3,4-dihydroisoquinolin-6-yl)-1-piperidyl]acetamide (0.5 g, crude), which was used for the next step without purification.

LCMS(ESI): [M+H]⁺ m/z: calcd 560.4; found 560.4; Rt=0.691 min.

### Step 11: The Synthesis of N-(4-Amino-2H-pyrazolo[4,3-c]pyridin-7-yl)-2-oxo-2-[rac-(2R,5S)-5-methyl-2-(1,1,2-trimethyl-3,4-dihydroisoquinolin-6-yl)-1-piperidyl]acetamide (Compound 221)

To a solution of N-(4-amino-2-tetrahydropyran-2-yl-pyrazolo[4,3-c]pyridin-7-yl)-2-oxo-2-[rac-(2R,5S)-5-methyl-2-(1,1,2-trimethyl-3,4-dihydroisoquinolin-6-yl)-1-piperidyl]acetamide (0.5 g, 893.33 μmol) in Methanol (5 mL), Hydrogen chloride, 4M in 1,4-dioxane, 99% (4 g, 109.71 mmol, 5.00 mL) was added. The resulting mixture was stirred at 25° C. for 12 hr and evaporated in vacuo. The residue was purified by HPLC ((Mobile Phase, Column): SYSTEM 0-0-20% 0-1.5-5 min H₂O/ACN/0.1% FA, flow: 30 mL/min (loading pump 4 mL/min acetonitrile); column: Chromatorex 18 SMB100-5T 100×19 mm 5 um) to obtain N-(4-amino-2H-pyrazolo[4,3-c]pyridin-7-yl)-2-oxo-2-[rac-(2R,5S)-5-methyl-2-(1,1,2-trimethyl-3,4-dihydroisoquinolin-6-yl)-1-piperidyl]acetamide (90 mg, 172.54 μmol, 19.31% yield, HCO₂H).

¹H NMR (500 MHz, dmso) δ 0.73-1.07 (m, 3H), 1.19-1.34 (m, 1H), 1.36-1.46 (m, 6H), 1.61-1.79 (m, 1H), 1.80-2.01 (m, 1H), 2.08-2.28 (m, 1H), 2.71-2.83 (m, 1H), 2.84-2.94 (m, 2H), 2.94-3.08 (m, 3H), 3.45-3.54 (m, 2H), 3.69-4.06 (m, 2H), 4.87-5.71 (m, 1H), 6.66-6.77 (m, 1H), 6.98-7.09 (m, 1H), 7.09-7.20 (m, 1H), 7.30-7.42 (m, 1H), 7.52-7.82 (m, 1H), 8.10-8.23 (m, 2H), 8.26-10.63 (m, 1H), 12.77 (s, 1H).

LCMS(ESI): [M+2H]⁺ m/z: calcd 477.2; found 477.2; Rt=0.727 min.

### Example 198. N-(4-Amino-1H-pyrazolo[4,3-c]pyridin-7-yl)-2-oxo-2-[(2R,5S)-5-methyl-2-[rel-(2R)-tetrahydropyran-2-yl]-1-piperidyl]acetamide (Compound 232) and N-(4-Amino-1H-pyrazolo[4,3-c]pyridin-7-yl)-2-oxo-2-[(2R,5S)-5-methyl-2-[rel-(2S)-tetrahydropyran-2-yl]-1-piperidyl]acetamide (Compound 235)

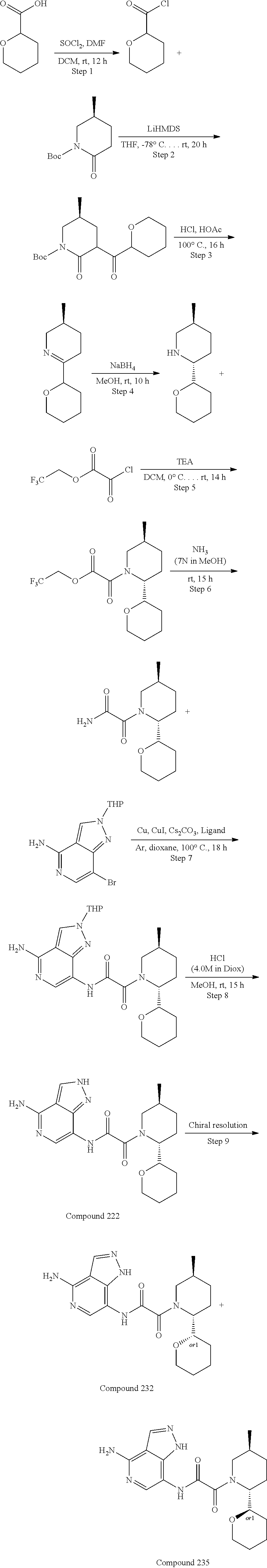

### Step 1: The Synthesis of Tetrahydro-2H-pyran-2-carbonyl chloride

Thionyl chloride (3.84 g, 32.27 mmol, 2.34 mL) was added dropwise to a mixture of tetrahydropyran-2-carboxylic acid (3.5 g, 26.89 mmol) and DMF (98.28 mg, 1.34 mmol, 104.11 μL) in DCM. Resulting solution was stirred at 25° C. for 12 hr. The solvent was evaporated to dryness to obtain tetrahydropyran-2-carbonyl chloride (3.9 g, crude).

¹H NMR (400 MHZ, CDCl₃) δ (ppm) 1.60 (m, 3H), 1.76 (m, 1H), 1.89 (m, 1H), 2.13 (m, 1H), 3.53 (m, 1H), 4.05 (m, 1H), 4.25 (m, 1H).

### Step 2: The Synthesis of tert-Butyl (5S)-5-methyl-2-oxo-3-(tetrahydropyran-2-carbonyl)piperidine-1-carboxylate

At −78° C., Lithium bis(trimethylsilyl)amide (4.71 g, 28.13 mmol) was added dropwise to a solution of tert-butyl (5S)-5-methyl-2-oxo-piperidine-1-carboxylate (5 g, 23.44 mmol) in THF (100 mL) under Ar flow. The resulting mixture was stirred at −78° C. for 1 hr. Then the solution of tetrahydropyran-2-carbonyl chloride (3.48 g, 23.44 mmol) in THF was added at −78° C. to the previous mixture. After 30 min the solution was allowed warm to rt and stirred overnight. The reaction mixture was quenched with NaHSO₄ (2 g; 10% solution) and extracted with DCM (3*50 ml). Organic layers was washed with water, dried over Na₂SO₄. DCM was evaporated to give tert-butyl (5S)-5-methyl-2-oxo-3-(tetrahydropyran-2-carbonyl)piperidine-1-carboxylate (9.18 g, crude).

LCMS(ESI): [M-Boc]⁺ m/z: calcd 226.2; found 226.2; Rt=1.446 min.

### Step 3: The Synthesis of (3S)-3-Methyl-6-tetrahydropyran-2-yl-2,3,4,5-tetrahydropyridine

A solution of tert-butyl (5S)-5-methyl-2-oxo-3-(tetrahydropyran-2-carbonyl)piperidine-1-carboxylate (9.18 g, 28.21 mmol) in HCl (conc) (30 mL) and Glacial acetic acid (30 mL) was heated at 100° C. for 16 hr.

The reaction mixture was warmed to rt and the basified to pH=10 by sat. Na₂CO₃ aq. solution and then was extracted with DCM (3×20 ml). Organic phase was dried over Na₂SO₄, filtered and evaporated to obtain (3S)-3-methyl-6-tetrahydropyran-2-yl-2,3,4,5-tetrahydropyridine (2.87 g, crude)

LCMS(ESI): [M+H]⁺ m/z: calcd 182.2; found 182.2; Rt=0.525 min.

### Step 4: The Synthesis of (2R,5S)-5-methyl-2-tetrahydropyran-2-yl-piperidine

Sodium Borohydride (598.94 mg, 15.83 mmol, 557.67 μL) was added in one portion to a stirred solution of (3S)-3-methyl-6-tetrahydropyran-2-yl-2,3,4,5-tetrahydropyridine (2.87 g, 15.83 mmol) in Methanol (30 mL) at 25° C. The resulting mixture was stirred at 25° C. for 10 hr, and then evaporated in vacuo. The residue was diluted with water (150 mL) and extracted with dichloromethane (2*150 mL). The combined organic extracts were dried over sodium sulfate and evaporated in vacuo. Resulting crude product was diluted with water (100 ml) and acidified to pH=3 with NaHSO₄, resulting mixture was washed with DCM (2×100 ml). Aqeuous solution was basified with potassium carbonate to pH=9 and extracted with DCM (2×100). Organic phase was separated, dried over Na₂SO₄, filtered and evaporated to obtain (2R,5S)-5-methyl-2-tetrahydropyran-2-yl-piperidine (1.76 g, crude).

LCMS(ESI): [M+H]⁺ m/z: calcd 184.2; found 184.2; Rt=0.829 min.

### Step 5: The Synthesis of 2,2,2-Trifluoroethyl 2-oxo-2-[2R,5S)-5-methyl-2-tetrahydropyran-2-yl-1-piperidyl]acetate

To a stirred solution of 2R,5S)-5-methyl-2-tetrahydropyran-2-yl-piperidine (1.76 g, 9.60 mmol) in Dichloromethane (20 mL) were added triethylamine (1.46 g, 14.40 mmol, 2.01 mL) respectively at room temperature. The resulting reaction mixture was cooled to 0° C. Then 2,2,2-trifluoroethyl 2-chloro-2-oxo-acetate (1.83 g, 9.60 mmol) was added dropwise. The reaction was stirred extra 30 minutes at 0° C., then allowed warmed to room temperature and stirred 12 hr. Upon completion, the reaction mixture was washed with water (2×20 ml). Organic phase was then dried over Na₂SO₄, filtered and concentrated under reduced pressure to obtain crude product as brown gum. Product was used for the next step without purification. The reaction was successful. The desired product 2,2,2-trifluoroethyl 2-oxo-2-[(2R,5S)-5-methyl-2-tetrahydropyran-2-yl-1-piperidyl]acetate (3.15 g, crude) was isolated as a brown gum.

LCMS(ESI): [M+H]⁺ m/z: calcd 338.2; found 338.4; Rt=1.387 min.

### Step 6: The Synthesis of 2-Oxo-2-[(2R,5S)-5-methyl-2-tetrahydropyran-2-yl-1-piperidyl]acetamide

A solution of 2,2,2-trifluoroethyl 2-oxo-2-[2R,5S)-5-methyl-2-tetrahydropyran-2-yl-1-piperidyl]acetate (3.15 g, 9.34 mmol) in Methanol/NH₃ (7N) (15 mL) was stirred at 25° C. for 15 hr. The solvent was evaporated to obtain crude 1.8 2-oxo-2-[2R,5S)-5-methyl-2-tetrahydropyran-2-yl-1-piperidyl]acetamide (0.8 g, 3.15 mmol, 33.69% yield), 1 g of the material was purified by HPLC (0-2-9 min 18-25-40% meoh/h2o+nh40 h 30 ml/min (loading pump 4 ml meoh), target mass 2551 column: Chromatorex C18 SMB100-5T 100*19 mm, 5 microM) to obtain 2-oxo-2-[2R,5S)-5-methyl-2-tetrahydropyran-2-yl-1-piperidyl]acetamide (198.4 mg, crude).

LCMS(ESI): [M+H]⁺ m/z: calcd 255.2; found 255.2; Rt=1.125 min.

### Step 7: The Synthesis of N-(4-Amino-2-tetrahydropyran-2-yl-pyrazolo[4,3-c]pyridin-7-yl)-2-oxo-2-[(2R,5S)-5-methyl-2-tetrahydropyran-2-yl-1-piperidyl]acetamide

2-oxo-2-[(2R,5S)-5-methyl-2-tetrahydropyran-2-yl-1-piperidyl]acetamide (198.4 mg, 780.11 μmol), 4-bromo-2-tetrahydropyran-2-yl-pyrazolo[3,4-c]pyridin-7-amine (231.81 mg, 780.11 μmol), Copper (2.48 mg, 39.01 μmol), Copper (I) iodide (74.29 mg, 390.05 μmol, 13.22 μL), rac-(1R,2R)—N1,N2-dimethylcyclohexane-1,2-diamine (83.22 mg, 585.08 μmol) and Cesium carbonate (508.35 mg, 1.56 mmol) were mixed in Dioxane (5 mL). The reaction mixture was purged with argon for 5 min. Vials were sealed and heated at 25° C. for 15 hr. The reaction mixture was cooled and filtered. The filter cake was rinsed with MeOH (10 ml) and the filtrate was concentrated in vacuo to obtain N-(4-amino-2-tetrahydropyran-2-yl-pyrazolo[4,3-c]pyridin-7-yl)-2-oxo-2-[(2R,5S)-5-methyl-2-tetrahydropyran-2-yl-1-piperidyl]acetamide (456 mg, crude) which was used in the next step without purification.

LCMS(ESI): [M+H]⁺ m/z: calcd 471.2; found 471.2; Rt=1.204 min.

### Step 8: The Synthesis of N-(4-Amino-1H-pyrazolo[4,3-c]pyridin-7-yl)-2-oxo-2-[2R,5S)-5-methyl-2-tetrahydropyran-2-yl-1-piperidyl]acetamide (Compound 222)

N-(4-amino-2-tetrahydropyran-2-yl-pyrazolo[4,3-c]pyridin-7-yl)-2-oxo-2-[2R,5S)-5-methyl-2-tetrahydropyran-2-yl-1-piperidyl]acetamide (456 mg, 969.05 μmol) was dissolved in Dioxane/HCl (2 mL) and Methanol (2 mL). Resulting solution was stirred at rt for 15 hr. Solvents were evaporated. Resulting crude product was purified by HPLC (3-10-55% 0-2-10 minH2O/ACN/0.1NH4OH, flow 30 ml/min ((loading pump 4 mL ACN) target mass 387 column: XBridge BEH C18 100*19 mm, 5 microM) and then repurified by HPLC (0-70% 0-2-10 min H2O/MEOH/0.1FA, flow 30 ml/min ((loading pump 4 ml MEOH) target mass 387 column: Chromatorex C18 SMB100-5T 100*19 mm, 5 microM) to obtain N-(4-amino-1H-pyrazolo[4,3-c]pyridin-7-yl)-2-oxo-2-[-(2R,5S)-5-methyl-2-tetrahydropyran-2-yl-1-piperidyl]acetamide (41.2 mg, 106.61 μmol, 11.00% yield).

LCMS(ESI): [M+H]⁺ m/z: calcd 387.2; found 387.4; Rt=0.922 min.

### Step 9: The Synthesis of N-(4-Amino-1H-pyrazolo[4,3-c]pyridin-7-yl)-2-oxo-2-[(2R,5S)-5-methyl-2-[rel-(2R)-tetrahydropyran-2-yl]-1-piperidyl]acetamide (Compound 232) and N-(4-Amino-1H-pyrazolo[4,3-c]pyridin-7-yl)-2-oxo-2-[(2R,5S)-5-methyl-2-[rel-(2S)-tetrahydropyran-2-yl]-1-piperidyl]acetamide (Compound 235)

N-(4-Amino-1H-pyrazolo[4,3-c]pyridin-7-yl)-2-oxo-2-[(2R,5S)-5-methyl-2-tetrahydropyran-2-yl-1-piperidyl]acetamide was chirally separated (Column: Chiralpak IC (250*20 mm, 5 mkm); Mobile phase: Hexane (0.1% DEA)-IPA-MeOH, 70-15-15. Flow Rate: 12 mL/min; Column Temperature: 24° C.; Wavelength: 205 nm. RetTime (isomer A)=19.812 min; RetTime (isomer B)=29.487 min) to obtain N-(4-amino-1H-pyrazolo[4,3-c]pyridin-7-yl)-2-oxo-2-[(2R,5S)-5-methyl-2-[rel-(2R)-tetrahydropyran-2-yl]-1-piperidyl]acetamide (18.45 mg, 47.74 μmol) (RetTime (Compound 232)=19.812 min) and N-(4-amino-1H-pyrazolo[4,3-c]pyridin-7-yl)-2-oxo-2-[(2R,5S)-5-methyl-2-[rel-(2S)-tetrahydropyran-2-yl]-1-piperidyl]acetamide (7.37 mg, 19.07 μmol) (RetTime (Compound 235)=29.487 min)

### Preparative:

Column: Chiralpak IC (250*20 mm, 5 mkm); Mobile phase: Hexane (0.1% DEA)-IPA-MeOH, 70-15-15. Flow Rate: 12 mL/min; Column Temperature: 24° C.; Wavelength: 205 nm.

RetTime (Compound 232)=19.812 min;

RetTime (Compound 235)=29.487 min

### Analytical:

Column: Chiralpak IC (250×4.6 mm, 5 mkm)-2; Mobile Phase: Hexane (0.1% EDA):MeOH:IPA, 50:25:25; Flow Rate: 0.6 ml/min.

RetTime (Compound 232)=9.763 min;

RetTime (Compound 235)=12.571 min

### Compound 232:

¹H NMR (600 MHz, dmso) δ 0.81-0.99 (m, 3H), 1.09-1.66 (m, 7H), 1.67-2.02 (m, 4H), 2.68-3.21 (m, 1H), 3.36-4.35 (m, 5H), 6.59-6.81 (m, 2H), 7.37-7.78 (m, 1H), 8.09-8.25 (m, 1H), 9.89 (s, 1H), 12.54-13.42 (m, 1H).

LCMS(ESI): [M+H]⁺ m/z: calcd 387.2; found 387.2; Rt=2.371 min.

### Compound 235:

¹H NMR (600 MHz, dmso) δ 0.91-0.98 (m, 3H), 1.17-1.32 (m, 2H), 1.32-1.69 (m, 5H), 1.70-2.04 (m, 4H), 2.87-3.13 (m, 1H), 3.44-4.04 (m, 4H), 4.07-4.41 (m, 1H), 6.56-6.78 (m, 2H), 7.39-7.84 (m, 1H), 8.08-8.23 (m, 1H), 9.23-10.40 (m, 1H), 12.44-13.31 (m, 1H).

LCMS(ESI): [M+H]⁺ m/z: calcd 387.2; found 387.2; Rt=2.394 min.

### Example 199. N-(4-Amino-1H-pyrazolo[4,3-c]pyridin-7-yl)-2-oxo-2-[rac-(2S,5R)-5-ethyl-2-methyl-1-piperidyl]acetamide (Compound 223) and N-(4-Amino-1H-pyrazolo[4,3-c]pyridin-7-yl)-2-oxo-2-[rac-(2R,5R)-5-ethyl-2-methyl-1-piperidyl]acetamide (Compound 269)

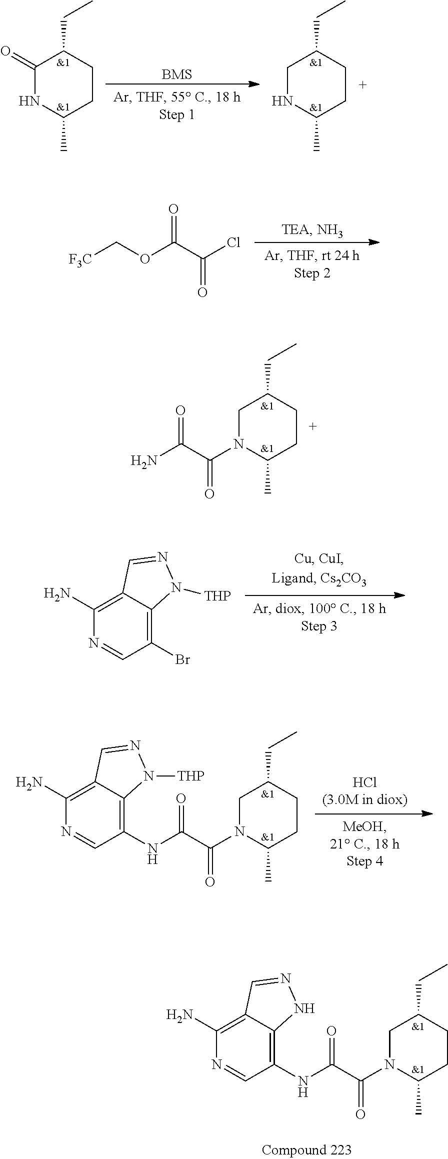

### Step 1: The Synthesis of rac-(2S,5R)-5-Ethyl-2-methyl-piperidine

To a solution of rac-(3R,6S)-3-ethyl-6-methyl-piperidin-2-one (0.5 g, 3.54 mmol) in THF (5 mL), borane-dimethyl sulfide (1.34 g, 17.70 mmol, 1.68 mL) in THF (5 mL) was slowly added at 0° C. The reaction mixture was stirred under argon at 55° C. for 18 hr. The reaction mixture was cooled to room temperature and poured slowly into methanol. After stirring at reflux for 30 min, solvent was removed under reduced pressure. The residue was quenched with hydrochloric acid (20% aq.) and concentrated in vacuo to give rac-(2S,5R)-5-ethyl-2-methyl-piperidine (0.5 g, crude, HCl).

### Step 2: The Synthesis of 2-Oxo-2-[rac-(2S,5R)-5-ethyl-2-methyl-1-piperidyl]acetamide

To a solution of rac-(2S,5R)-5-ethyl-2-methyl-piperidine (500.00 mg, 3.05 mmol, HCl) and TEA (618.19 mg, 6.11 mmol, 851.50 μL) in THF (10 mL) was added 2,2,2-trifluoroethyl 2-chloro-2-oxo-acetate (872.88 mg, 4.58 mmol) dropwise at 0° C. under argon. The reaction mixture was then stirred for 12 hr at r.t. Then ammonia (292.20 mg, 17.16 mmol) was bubbled through for 10 min at 0° C. The reaction mixture was then stirred for 12 hr at r.t. The reaction mixture was filtered off and the filtrate was evaporated in vacuo to give 2-oxo-2-[rac-(2S,5R)-5-ethyl-2-methyl-1-piperidyl]acetamide (0.4 g, 2.02 mmol, 66.05% yield).

LCMS(ESI): [M+H]⁺ m/z: calcd 199.2; found 199.2; Rt=0.947 min.

### Step 3: The Synthesis of N-(4-Amino-2-tetrahydropyran-2-yl-pyrazolo[4,3-c]pyridin-7-yl)-2-oxo-2-[rac-(2S,5R)-5-ethyl-2-methyl-1-piperidyl]acetamide

2-Oxo-2-[rac-(2S,5R)-5-ethyl-2-methyl-1-piperidyl]acetamide (0.4 g, 2.02 mmol), 7-bromo-2-tetrahydropyran-2-yl-pyrazolo[4,3-c]pyridin-4-amine (899.27 mg, 3.03 mmol), Cu (13.59 mg, 213.86 μmol), CuI (115.27 mg, 605.26 μmol, 20.51 μL), Caesium carbonate (986.03 mg, 3.03 mmol, 430.58 μL) and (1R,2R)—N1,N2-dimethylcyclohexane-1,2-diamine (86.09 mg, 605.26 μmol) were mixed in dioxane (8 mL), purged with Ar for 2 minutes and then heated in the sealed tube at 100° C. for 18 hr. Final mixture was filtered and the filtrate was evaporated in vacuo to afford N-(4-amino-2-tetrahydropyran-2-yl-pyrazolo[4,3-c]pyridin-7-yl)-2-oxo-2-[rac-(2S,5R)-5-ethyl-2-methyl-1-piperidyl]acetamide (1 g, crude) as brown solid, which was used directly in the next step.

LCMS(ESI): [M+H]⁺ m/z: calcd 415.2; found 415.2; Rt=0.997 min.

### Step 4: The Synthesis of N-(4-Amino-1H-pyrazolo[4,3-c]pyridin-7-yl)-2-oxo-2-[rac-(2S,5R)-5-ethyl-2-methyl-1-piperidyl]acetamide (Compound 223)

To a solution of N-(4-amino-2-tetrahydropyran-2-yl-pyrazolo[4,3-c]pyridin-7-yl)-2-oxo-2-[rac-(2S,5R)-5-ethyl-2-methyl-1-piperidyl]acetamide (1.00 g, 579.01 μmol) in methanol (10 mL) was added Hydrogen chloride solution 3.0M in dioxane (4.22 g, 11.58 mmol, 4.02 mL, 10% purity) at 21° C. The resulting mixture was left to stir for 18 hr. The resulting mixture was evaporated to dryness. The residue was purified by RP-HPLC (column: Chromatorex 18 SMB100-5T 100×19 mm 5 um; 5-30% 0-5 min H₂O/ACN/0.1% FA, flow: 30 mL/min) to give Compound 223 N-(4-amino-1H-pyrazolo[4,3-c]pyridin-7-yl)-2-oxo-2-[rac-(2S,5R)-5-ethyl-2-methyl-1-piperidyl]acetamide (68 mg, 180.65 μmol, 31.20% yield, HCO₂H).

¹H NMR (500 MHz, dmso) δ 0.73-0.91 (m, 3H), 1.13-1.45 (m, 8H), 1.53-1.71 (m, 3H), 3.68-3.84 (m, 1H), 4.21-4.29 (m, 1H), 6.68-6.82 (m, 2H), 7.64-7.79 (m, 1H), 8.16-8.26 (m, 1H), 10.03-10.69 (m, 1H), 12.09-13.47 (m, 2H).

LCMS(ESI): [M+H]⁺ m/z: calcd 331.2; found 331.2; Rt=1.977 min.

The Synthesis of N-(4-Amino-1H-pyrazolo[4,3-c]pyridin-7-yl)-2-oxo-2-[rac-(2R,5R)-5-ethyl-2-methyl-1-piperidyl]acetamide (Compound 269)

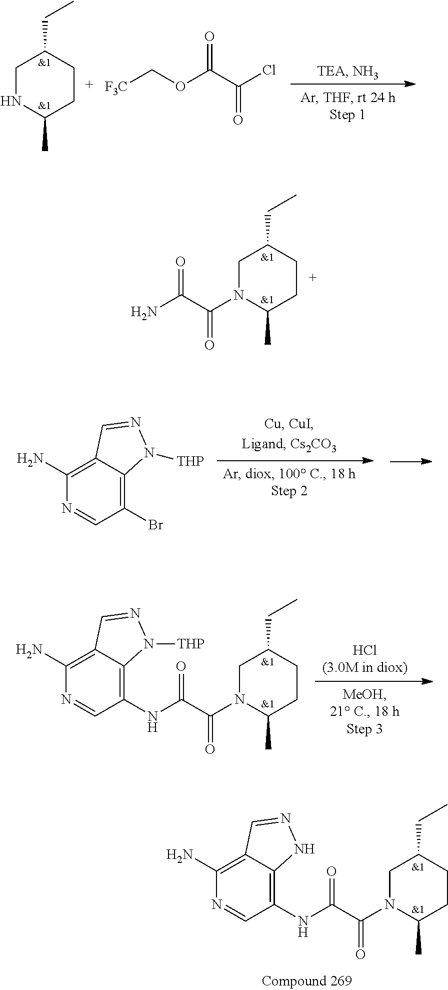

### Step 1: The Synthesis of 2-Oxo-2-[rac-(2R,5R)-5-ethyl-2-methyl-1-piperidyl]acetamide

To a solution of rac-(2R,5R)-5-ethyl-2-methyl-piperidine (0.5 g, 3.93 mmol) and TEA (795.35 mg, 7.86 mmol, 1.10 mL) in THF (53.68 mL) was added 2,2,2-trifluoroethyl 2-chloro-2-oxo-acetate (1.12 g, 5.89 mmol) dropwise at 0° C. under argon. The reaction mixture was then stirred for 12 hr at r.t. Then ammonia (292.20 mg, 17.16 mmol) was bubbled through for 10 min at 0° C. The reaction mixture was then stirred for 12 hr at r.t. The reaction mixture was filtered off and the filtrate was evaporated in vacuo to give 2-oxo-2-[rac-(2R,5R)-5-ethyl-2-methyl-1-piperidyl]acetamide (0.5 g, 2.52 mmol, 64.17% yield).

LCMS(ESI): [M+H]⁺ m/z: calcd 199.2; found 199.2; Rt=0.801 min.

### Step 2: The Synthesis of N-(4-Amino-2-tetrahydropyran-2-yl-pyrazolo[4,3-c]pyridin-7-yl)-2-oxo-2-[rac-(2R,5R)-5-ethyl-2-methyl-1-piperidyl]acetamide

2-Oxo-2-[rac-(2R,5R)-5-ethyl-2-methyl-1-piperidyl]acetamide (150 mg, 756.58 μmol), 7-bromo-2-tetrahydropyran-2-yl-pyrazolo[4,3-c]pyridin-4-amine (337.23 mg, 1.13 mmol), Cu (5.10 mg, 80.20 μmol), CuI (43.23 mg, 226.97 μmol, 7.69 μL), Caesium carbonate (369.76 mg, 1.13 mmol, 161.47 μL) and (1R,2R)—N1,N2-dimethylcyclohexane-1,2-diamine (32.28 mg, 226.97 μmol) were mixed in dioxane (5 mL), purged with Ar for 2 minutes and then heated in the sealed tube at 100° C. for 18 hr. Final mixture was filtered and the filtrate was evaporated in vacuo to afford N-(4-amino-2-tetrahydropyran-2-yl-pyrazolo[4,3-c]pyridin-7-yl)-2-oxo-2-[rac-(2R,5R)-5-ethyl-2-methyl-1-piperidyl]acetamide (0.5 g, crude) as brown solid, which was used directly in the next step.

LCMS(ESI): [M+H]⁺ m/z: calcd 415.2; found 415.2; Rt=0.799 min.

### Step 3: The Synthesis of N-(4-Amino-1H-pyrazolo[4,3-c]pyridin-7-yl)-2-oxo-2-[rac-(2R,5R)-5-ethyl-2-methyl-1-piperidyl]acetamide (Compound 269)

To a solution of N-(4-amino-2-tetrahydropyran-2-yl-pyrazolo[4,3-c]pyridin-7-yl)-2-oxo-2-[rac-(2R,5R)-5-ethyl-2-methyl-1-piperidyl]acetamide (500.00 mg, 723.76 μmol) in methanol (10 mL) was added Hydrogen chloride solution 3.0M in dioxane (5.28 g, 14.48 mmol, 5.03 mL, 10% purity) at 21° C. The resulting mixture was left to stir for 18 hr. The resulting mixture was evaporated to dryness. The residue was purified by RP-HPLC (column: XBridge OBD C18 100×30 mm; 30-80% 0-1-5 min H₂O/MeOH/NH₄OH flow: 30 mL/min) to give Compound 269 N-(4-amino-1H-pyrazolo[4,3-c]pyridin-7-yl)-2-oxo-2-[rac-(2R,5R)-5-ethyl-2-methyl-1-piperidyl]acetamide (50 mg, 151.34 μmol, 20.91% yield).

¹H NMR (500 MHz, dmso) δ 0.22-0.97 (m, 4H), 1.05-1.28 (m, 4H), 1.32-1.93 (m, 5H), 2.93-3.41 (m, 1H), 3.64-4.69 (m, 2H), 6.48-6.84 (m, 2H), 7.30-7.77 (m, 1H), 8.05-8.25 (m, 1H).

LCMS(ESI): [M+H]⁺ m/z: calcd 331.2; found 331.2; Rt=2.125 min.

### Example 200. N-(4-Amino-1H-pyrazolo[4,3-c]pyridin-7-yl)-2-oxo-2-[rac-(2R,5S)-2-[1-[2-(dimethylamino)ethyl]pyrazol-3-yl]-5-methyl-1-piperidyl]acetamide (Compound 224)

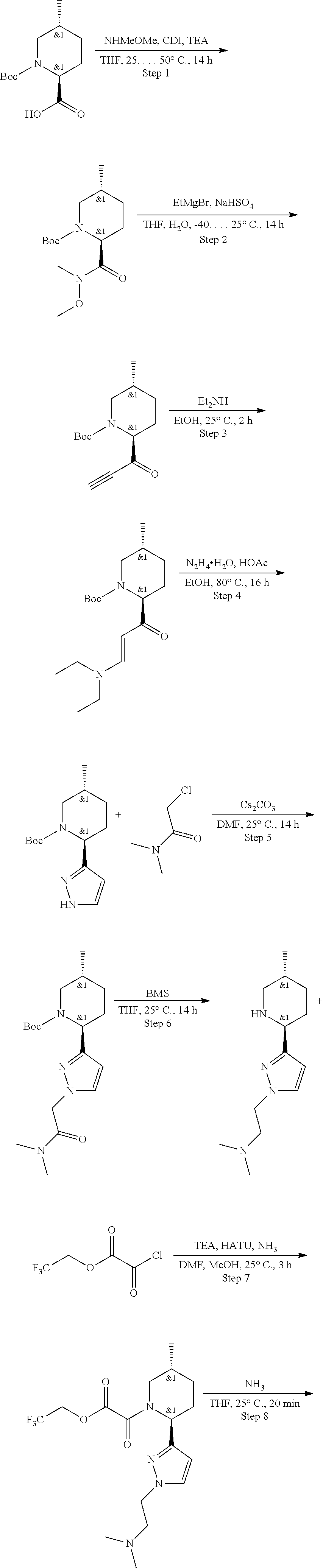

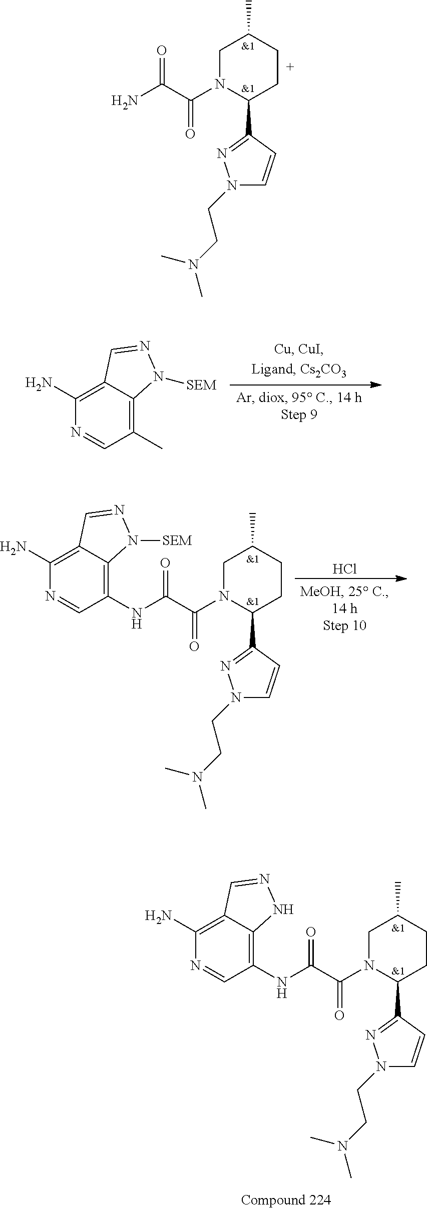 [SMILES: C[C@@H]1CC[C@@H](c2ccn(CCN(C)C)n2)N(C(=O)C(N)=O)C1 ; Cc1cnc(N)c2cnn(COCC[Si](C)(C)C)c12 ; C[C@@H]1CC[C@@H](c2ccn(CCN(C)C)n2)N(C(=O)C(=O)Nc2cnc(N)c3cnn(COCC[Si](C)(C)C)c23)C1 ; Cl ; C[C@@H]1CC[C@@H](c2ccn(CCN(C)C)n2)N(C(=O)C(=O)Nc2cnc(N)c3cn[nH]c23)C1]

### Step 1: The Synthesis of tert-Butyl rac-(2R,5S)-2-[methoxy(methyl) carbamoyl]-5-methyl-piperidine-1-carboxylate

Carbonyldiimidazole (1.60 g, 9.86 mmol) was added in one portion to a stirred solution of rac-(2R,5S)-1-tert-butoxycarbonyl-5-methyl-piperidine-2-carboxylic acid (2.00 g, 8.22 mmol) in THF (22.71 mL) at 25° C. The resulting mixture was stirred at 45° C. until carbon dioxide evolution was completed, then Triethylamine (1.66 g, 16.44 mmol, 2.29 mL) and N-methoxymethanamine (1.44 g, 14.80 mmol, HCl) were added. The reaction mixture was stirred at 50° C. for 14 hr, then cooled and evaporated in vacuo. The residue was diluted with 5% aqueous sodium hydrogen sulphate solution (70 ml) and extracted with dichloromethane (2*100 mL). The combined organic extracts were washed with water (100 ml), dried over sodium sulphate and evaporated in vacuo to afford tert-butyl rac-(2R,5S)-2-[methoxy(methyl) carbamoyl]-5-methyl-piperidine-1-carboxylate (2.3 g, 8.03 mmol, 97.70% yield).

### Step 2: The Synthesis of tert-Butyl rac-(2R,5S)-5-methyl-2-prop-2-ynoyl-piperidine-1-carboxylate

A solution of tert-butyl rac-(2R,5S)-2-[methoxy(methyl) carbamoyl]-5-methyl-piperidine-1-carboxylate (2.30 g, 8.03 mmol) in THF (20 mL) was added dropwise at −40° C. to a stirred solution of Ethynylmagnesium bromide (5.19 g, 40.16 mmol, 80 mL) (0.5M in THF solution). The resulting mixture was allowed to warm to 25° C. and stirred at this temperature for 14 hr, then poured into Sodium bisulfate (9.64 g, 80.32 mmol) in Water (100 mL). The resulting mixture was stirred for 2 h, then transferred into a separatory funnel. The upper organic layer was separated, the aqueous layer was additionally extracted with MTBE (2*50 mL). The combined organic extracts were washed with brine (2*100 mL), dried over sodium sulphate and evaporated in vacuo to give tert-butyl rac-(2R,5S)-5-methyl-2-prop-2-ynoyl-piperidine-1-carboxylate (2 g, 7.96 mmol, 99.08% yield).

LCMS(ESI): [M-Boc]⁺ m/z: calcd 152.2; found 152.2; Rt=1.385 min.

### Step 3: The Synthesis of tert-Butyl rac-(2R,5S)-5-methyl-2-[rac-(E)-3-(diethylamino) prop-2-enoyl]piperidine-1-carboxylate

tert-Butyl rac-(2R,5S)-5-methyl-2-prop-2-ynoyl-piperidine-1-carboxylate (2.00 g, 7.96 mmol) was diluted with a solution of N-ethylethanamine (582.02 mg, 7.96 mmol, 824.39 μL) in Ethanole (50.00 mL). The resulting solution was stirred at 25° C. for 1.5 hr, and then evaporated in vacuo to give tert-butyl rac-(2R,5S)-5-methyl-2-[rac-(E)-3-(diethylamino) prop-2-enoyl]piperidine-1-carboxylate (2.5 g, 7.71 mmol, 96.82% yield).

LCMS(ESI): [M+H]⁺ m/z: calcd 325.2; found 325.2; Rt=1.392 min.

### Step 4: The Synthesis of tert-Butyl rac-(2R,5S)-5-methyl-2-(1H-pyrazol-3-yl)piperidine-1-carboxylate

Hydrazine; hydrate (740.59 mg, 14.79 mmol, 721.12 μL) was added to a stirred solution of tert-butyl rac-(2R,5S)-5-methyl-2-[rac-(E)-3-(diethylamino) prop-2-enoyl]piperidine-1-carboxylate (2.40 g, 7.40 mmol) and Acetic acid (888.41 mg, 14.79 mmol, 846.91 μL) in Ethanol (100 mL). The reaction mixture was stirred with reflux condenser at 80° C. for 16 hr, then cooled and evaporated in vacuo. The residue was diluted with water (100 mL) and extracted with MTBE (2*100 mL). The combined organic extracts were washed with water (50 mL), dried over sodium sulphate and evaporated in vacuo to give tert-butyl rac-(2R,5S)-5-methyl-2-(1H-pyrazol-3-yl)piperidine-1-carboxylate (1.9 g, 7.16 mmol, 96.80% yield).

LCMS(ESI): [M-Boc]⁺ m/z: calcd 166.2; found 166.2; Rt=1.257 min.

### Step 5: The Synthesis of tert-Butyl rac-(2R,5S)-2-[1-[2-(dimethylamino)-2-oxo-ethyl]pyrazol-3-yl]-5-methyl-piperidine-1-carboxylate

To the stirred solution of tert-butyl rac-(2R,5S)-5-methyl-2-(1H-pyrazol-3-yl)piperidine-1-carboxylate (800.00 mg, 3.01 mmol) in DMF (5 mL) Cesium carbonate (1.47 g, 4.52 mmol) was added followed by addition of 2-chloro-N,N-dimethyl-acetamide (439.81 mg, 3.62 mmol, 372.09 μL). The resulting mixture was stirred at 25° C. for 14 hr. The reaction mixture was poured into water (30 mL). Product was extracted with MTBE (3*50 mL). Combined organic layers were dried over Na₂SO₄. MTBE was evaporated in vacuo to give crude product which was purified by reverse phase HPLC (SYSTEM 40-90% 0-5 min H₂O/MeOH, flow: 30 mL/min (loading pump 4 mL/min MeOH); column: Chromatorex 18 SMB100-5T 100×19 mm 5 um) to give tert-butyl rac-(2R,5S)-2-[1-[2-(dimethylamino)-2-oxo-ethyl]pyrazol-3-yl]-5-methyl-piperidine-1-carboxylate (0.68 g, 1.94 mmol, 64.36% yield).

LCMS(ESI): [M+H]⁺ m/z: calcd 351.2; found 351.2; Rt=1.311 min.

### Step 6: The Synthesis of N,N-Dimethyl-2-[3-[rac-(2R,5S)-5-methyl-2-piperidyl]pyrazol-1-yl]ethanamine

Borane dimethyl sulfide complex (86.71 mg, 1.14 mmol, 108.25 μL) was added dropwise to the solution of tert-butyl rac-(2R,5S)-2-[1-[2-(dimethylamino)-2-oxo-ethyl]pyrazol-3-yl]-5-methyl-piperidine-1-carboxylate (400.00 mg, 1.14 mmol) in THF (40 mL) under argon. Resulting mixture was stirred at 25° C. for 14 hr. Then, reaction was quenched by dropwise addition of methanol (10 mL). When H₂ evolution ceased, volatiles were removed under reduced pressure. The residue was diluted with 6M HCl (20 ml). The resulting mixture was stirred at 80° C. for 4 h. The reaction mixture was concentrated under reduced pressure. The residue was diluted with THF (50 mL). Precipitate which was formed was filtered, dried in vacuo to give N,N-dimethyl-2-[3-[rac-(2R,5S)-5-methyl-2-piperidyl]pyrazol-1-yl]ethanamine (0.4 g, crude).

LCMS(ESI): [M+H]⁺ m/z: calcd 237.4; found 237.4; Rt=0.165 min.

### Step 7: The Synthesis of 2,2,2-Trifluoroethyl 2-oxo-2-[rac-(2R,5S)-2-[1-[2-(dimethylamino)ethyl]pyrazol-3-yl]-5-methyl-1-piperidyl]acetate

To a solution of N,N-dimethyl-2-[3-[rac-(2R,5S)-5-methyl-2-piperidyl]pyrazol-1-yl]ethanamine (400.00 mg, 1.16 mmol, 3HCl) and Triethylamine (585.36 mg, 5.78 mmol, 806.28 μL) in THF (15 mL), 2,2,2-trifluoroethyl 2-chloro-2-oxo-acetate (661.21 mg, 3.47 mmol) was added portionwise at 25° C. The resulting mixture was stirred for 2 hr. LCMS showed full conversion of SM. The reaction mixture was directly used for the next step.

LCMS(ESI): [M+H]⁺ m/z: calcd 391.2; found 391.2; Rt=0.741 min.

### Step 8: The Synthesis of 2-Oxo-2-[rac-(2R,5S)-2-[1-[2-(dimethylamino)ethyl]pyrazol-3-yl]-5-methyl-1-piperidyl]acetamide

Through a solution of 2,2,2-trifluoroethyl 2-oxo-2-[rac-(2R,5S)-2-[1-[2-(dimethylamino)ethyl]pyrazol-3-yl]-5-methyl-1-piperidyl]acetate (450.00 mg, 1.15 mmol) in THF (25 mL), Ammonia (196.30 mg, 11.53 mmol) was bubbled during 20 min at 25° C. The formed precipitate was filtered off, washed with THF (30 mL) and the solvent was evaporated in vacuo to give 2-oxo-2-[rac-(2R,5S)-2-[1-[2-(dimethylamino)ethyl]pyrazol-3-yl]-5-methyl-1-piperidyl]acetamide (0.26 g, 845.83 μmol, 73.38% yield).

LCMS(ESI): [M+H]⁺ m/z: calcd 308.2; found 308.2; Rt=0.736 min.

### Step 9: The Synthesis of N-[4-Amino-1-(2-trimethylsilylethoxymethyl)pyrazolo[4,3-c]pyridin-7-yl]-2-oxo-2-[rac-(2R,5S)-2-[1-[2-(dimethylamino)ethyl]pyrazol-3-yl]-5-methyl-1-piperidyl]acetamide

A mixture of 2-oxo-2-[rac-(2R,5S)-2-[1-[2-(dimethylamino)ethyl]pyrazol-3-yl]-5-methyl-1-piperidyl]acetamide (260.00 mg, 845.83 μmol), 7-bromo-1-(2-trimethylsilylethoxymethyl)pyrazolo[4,3-c]pyridin-4-amine (290.37 mg, 845.83 μmol), Copper (I) iodide (161.09 mg, 845.83 μmol, 28.66 μL), Cesium carbonate (413.38 mg, 1.27 mmol), (1R,2R)—N1,N2-dimethylcyclohexane-1,2-diamine (180.47 mg, 1.27 mmol) and Cu (5.38 mg, 84.58 μmol) in Dioxane (4.01 mL) was stirred under argon at 95° C. for 14 hr. The reaction mixture was submitted for reverse phase HPLC (SYSTEM 40-40-90% 0-1-6 min H₂O/MeOH 0.1% NH₄OH, flow 30 mL/min (loading pump 4 mL/min ACN); column: XBridge BEH C18 5 um 130) to give N-[4-amino-1-(2-trimethylsilylethoxymethyl)pyrazolo[4,3-c]pyridin-7-yl]-2-oxo-2-[rac-(2R,5S)-2-[1-[2-(dimethylamino)ethyl]pyrazol-3-yl]-5-methyl-1-piperidyl]acetamide (71 mg, 124.61 μmol, 14.73% yield).

LCMS(ESI): [M+H]⁺ m/z: calcd 570.4; found 570.4; Rt=2.561 min.

### Step 10: The Synthesis of N-(4-Amino-1H-pyrazolo[4,3-c]pyridin-7-yl)-2-oxo-2-[rac-(2R,5S)-2-[1-[2-(dimethylamino)ethyl]pyrazol-3-yl]-5-methyl-1-piperidyl]acetamide (Compound 224)

To the stirred solution of N-[4-amino-1-(2-trimethylsilylethoxymethyl)pyrazolo[4,3-c]pyridin-7-yl]-2-oxo-2-[rac-(2R,5S)-2-[1-[2-(dimethylamino)ethyl]pyrazol-3-yl]-5-methyl-1-piperidyl]acetamide (71 mg, 124.61 μmol) in MeOH (1 mL) Hydrogen chloride solution 4.0M in dioxane (800.00 mg, 21.94 mmol, 1 mL) was added. The resulting mixture was stirred at 25° C. for 14 hr. The reaction mixture was concentrated under reduce pressure and was then purified by reverse phase HPLC (0-0-30% 0-1-6 min H₂O/ACN/0.1% NH₄OH, flow 30 mL/min (loading pump 4 mL/min ACN); column: XBridge BEH C18 5 um 130 A) to give N-(4-amino-1H-pyrazolo[4,3-c]pyridin-7-yl)-2-oxo-2-[rac-(2R,5S)-2-[1-[2-(dimethylamino)ethyl]pyrazol-3-yl]-5-methyl-1-piperidyl]acetamide (26 mg, 59.16 μmol, 47.47% yield).

¹H NMR (600 MHz, dmso) δ 0.84-1.04 (m, 3H), 1.20-1.42 (m, 1H), 1.58-2.00 (m, 3H), 2.06-2.14 (m, 7H), 2.56 (t, 1H), 2.60-2.65 (m, 1H), 2.84-3.21 (m, 1H), 3.65-4.05 (m, 1H), 4.06-4.18 (m, 2H), 4.90-5.67 (m, 1H), 5.99-6.23 (m, 1H), 6.59-6.82 (m, 2H), 7.44-7.74 (m, 2H), 8.09-8.22 (m, 1H), 9.51-10.54 (m, 1H), 12.59-13.41 (m, 1H).

LCMS(ESI): [M+H]⁺ m/z: calcd 440.4; found 440.4; Rt=1.642 min.

### Example 201. N-(4-Amino-1H-pyrazolo[4,3-c]pyridin-7-yl)-2-oxo-2-[rac-(2R,5S)-2-(5-methoxy-6-methyl-3-pyridyl)-5-methyl-1-piperidyl]acetamide (Compound 225)

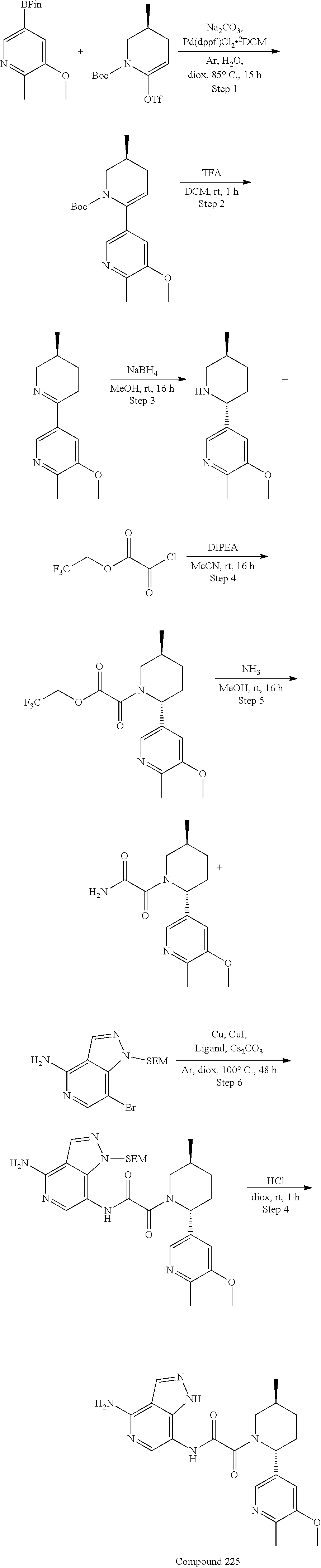

### Step 1: The Synthesis of tert-Butyl rac-(3S)-6-(5-methoxy-6-methyl-3-pyridyl)-3-methyl-3,4-dihydro-2H-pyridine-1-carboxylate

3-Methoxy-2-methyl-5-(4,4,5,5-tetramethyl-1,3,2-dioxaborolan-2-yl)pyridine (4.4 g, 17.66 mmol), tert-butyl rac-(3S)-3-methyl-6-(trifluoromethylsulfonyloxy)-3,4-dihydro-2H-pyridine-1-carboxylate (10.37 g, 30.03 mmol), Sodium carbonate (5.62 g, 52.99 mmol, 2.22 mL) and Pd(dppf)Cl2 DCM (721.20 mg, 883.13 μmol) were stirred in a mixture of 1,4-dioxane (40 mL) and water (15 mL) under inert atmosphere at 85° C. for 15 hr. Upon completion, the reaction mixture was cooled down, diluted with water and extracted with DCM. The organic layer was separated, dried over Na₂SO₄ and concentrated under reduced pressure to afford tert-butyl rac-(3S)-6-(5-methoxy-6-methyl-3-pyridyl)-3-methyl-3,4-dihydro-2H-pyridine-1-carboxylate (5 g, crude) which was used in the next step without purification.

LCMS(ESI): [M+H]⁺ m/z: calcd 319.2; found 319.2; Rt=1.096 min.

### Step 2: The Synthesis of 3-Methoxy-2-methyl-5-[rac-(3S)-3-methyl-2,3,4,5-tetrahydropyridin-6-yl]pyridine

tert-Butyl rac-(3S)-6-(5-methoxy-6-methyl-3-pyridyl)-3-methyl-3,4-dihydro-2H-pyridine-1-carboxylate (5 g, 15.70 mmol) was dissolved in a mixture of TFA (7.40 g, 64.90 mmol, 5 mL) and DCM (6 mL) and stirred at RT for 0.5 hr. Upon completion, the reaction mixture was concentrated under reduced pressure to afford 3-methoxy-2-methyl-5-[rac-(3S)-3-methyl-2,3,4,5-tetrahydropyridin-6-yl]pyridine (3.5 g, crude, TFA) which was used in the next step without purification.

LCMS(ESI): [M+H]⁺ m/z: calcd 219.2; found 219.2; Rt=0.743 min.

### Step 3: The Synthesis of 3-Methoxy-2-methyl-5-[rac-(2R,5S)-5-methyl-2-piperidyl]pyridine

3-Methoxy-2-methyl-5-[rac-(3S)-3-methyl-2,3,4,5-tetrahydropyridin-6-yl]pyridine (3.5 g, 10.56 mmol, TFA) was dissolved in methanol (30 mL) followed by portionwise addition of Sodium Borohydride (1.20 g, 31.69 mmol, 1.12 mL). The reaction mixture was stirred at RT overnight and concentrated under reduced pressure. The residue was treated with NaHCO₃ solution and extracted with MTBE. Organic layer was separated, dried over Na₂SO₄ and concentrated under reduced pressure to afford 3-methoxy-2-methyl-5-[rac-(2R,5S)-5-methyl-2-piperidyl]pyridine (3.8 g, 17.25 mmol, 163.27% yield) which was used in the next step without purification.

LCMS(ESI): [M+H]⁺ m/z: calcd 221.0; found 221.0; Rt=0.636 min.

### Step 4: The Synthesis of 2,2,2-Trifluoroethyl 2-oxo-2-[rac-(2R,5S)-2-(5-methoxy-6-methyl-3-pyridyl)-5-methyl-1-piperidyl]acetate

3-Methoxy-2-methyl-5-[rac-(2R,5S)-5-methyl-2-piperidyl]pyridine (3.8 g, 17.25 mmol) and DIPEA (6.69 g, 51.75 mmol, 9.01 mL) were dissolved in acetonitrile (60 mL) followed by dropwise addition of 2,2,2-trifluoroethyl 2-chloro-2-oxo-acetate (3.61 g, 18.97 mmol) at 0 C. The reaction mixture was stirred overnight and concentrated under reduced pressure. The residue was portioned between DCM and water. Organic layer was separated and concentrated. The residue was submitted to flash column chromatography to afford 2,2,2-trifluoroethyl 2-oxo-2-[rac-(2R,5S)-2-(5-methoxy-6-methyl-3-pyridyl)-5-methyl-1-piperidyl]acetate (1.9 g, 5.08 mmol, 29.43% yield).

Chromatography data: Companion combiflash, 80 g SiO₂, MTBE-MeOH from 0˜100%, flow rate=60 mL/min, cv=10

LCMS(ESI): [M+H]⁺ m/z: calcd 375.2; found 375.2; Rt=0.878 min.

### Step 5: The Synthesis of 2-Oxo-2-[rac-(2R,5S)-2-(5-methoxy-6-methyl-3-pyridyl)-5-methyl-1-piperidyl]acetamide

2,2,2-Trifluoroethyl 2-oxo-2-[rac-(2R,5S)-2-(5-methoxy-6-methyl-3-pyridyl)-5-methyl-1-piperidyl]acetate (1.9 g, 5.08 mmol) was dissolved in saturated ammonia/methanol solution and stirred overnight at RT. Upon completion the reaction mixture was concentrated under reduced pressure to afford 2-oxo-2-[rac-(2R,5S)-2-(5-methoxy-6-methyl-3-pyridyl)-5-methyl-1-piperidyl]acetamide (1.2 g, 4.12 mmol, 81.15% yield) which was used in the next step without purification.

LCMS(ESI): [M+H]⁺ m/z: calcd 292.2; found 292.; Rt=0.775 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-2-(5-methoxy-6-methyl-3-pyridyl)-5-methyl-1-piperidyl]acetamide

2-Oxo-2-[rac-(2R,5S)-2-(5-methoxy-6-methyl-3-pyridyl)-5-methyl-1-piperidyl]acetamide (0.4 g, 1.37 mmol), 7-bromo-1-(2-trimethylsilylethoxymethyl)pyrazolo[4,3-c]pyridin-4-amine (565.59 mg, 1.65 mmol), copper (0.015 g, 236.03 μmol), Copper (I) iodide (0.15 g, 787.61 μmol, 26.69 μL), (1S,2S)—N,N′-Bis-methyl-1,2-cyclohexane-diamine (195.29 mg, 1.37 mmol, 216.51 μL) were mixed in dioxane (10 mL). The reaction mixture was stirred under Ar atmosphere at 100° C. for 48 hr. Upon completion, the mixture concentrated under reduced pressure, treated with DMSO and filtered. The filtrate was submitted to HPLC to afford N-[4-amino-1-(2-trimethylsilylethoxymethyl)pyrazolo[4,3-c]pyridin-7-yl]-2-oxo-2-[rac-(2R,5S)-2-(5-methoxy-6-methyl-3-pyridyl)-5-methyl-1-piperidyl]acetamide (0.15 g, 270.89 μmol, 19.73% yield) in 2 fractions.

HPLC: n2-10 min 30-60% water-ACN+FA

LCMS(ESI): [M+H]⁺ m/z: calcd 554.4; found 554.4; Rt=1.117 min.

### Step 7: The Synthesis of N-(4-Amino-1H-pyrazolo[4,3-c]pyridin-7-yl)-2-oxo-2-[rac-(2R,5S)-2-(5-methoxy-6-methyl-3-pyridyl)-5-methyl-1-piperidyl]acetamide (Compound 225)

N-[4-Amino-1-(2-trimethylsilylethoxymethyl)pyrazolo[4,3-c]pyridin-7-yl]-2-oxo-2-[rac-(2R,5S)-2-(5-methoxy-6-methyl-3-pyridyl)-5-methyl-1-piperidyl]acetamide (0.15 g, 270.89 μmol) was dissolved in HCl/dioxane solution and stirred for 0.5 h at RT. Upon completion, the reaction mixture was concentrated and the residue was purified by HPLC to afford N-(4-amino-1H-pyrazolo[4,3-c]pyridin-7-yl)-2-oxo-2-[rac-(2R,5S)-2-(5-methoxy-6-methyl-3-pyridyl)-5-methyl-1-piperidyl]acetamide (0.05 g, 118.07 μmol, 43.59% yield).

HPLC: 2-10 min 20-60% water-methanol+FA; flow: 30 mL/min ((loading pump 4 mL methanol) column: SunFire 100*19, 5 microM

¹H NMR (600 MHz, dmso) δ 0.70-1.14 (m, 3H), 1.26-1.44 (m, 1H), 1.55-1.77 (m, 1H), 1.79-2.35 (m, 6H), 2.79-3.06 (m, 1H), 3.72-4.10 (m, 4H), 4.99-5.72 (m, 1H), 6.60-7.29 (m, 3H), 7.43-8.02 (m, 2H), 8.15-8.29 (m, 1H), 9.56-10.58 (m, 1H), 12.50-13.34 (m, 1H).

LCMS(ESI): [M+2H]⁺ m/z: calcd 425.2; found 425.2; Rt=1.786 min.

### Example 202. N-(4-Amino-1H-pyrazolo[4,3-c]pyridin-7-yl)-2-oxo-2-[rac-(2R,5S)-5-methyl-2-[1-(trifluoromethyl)cyclopropyl]-1-piperidyl]acetamide (Compound 226)

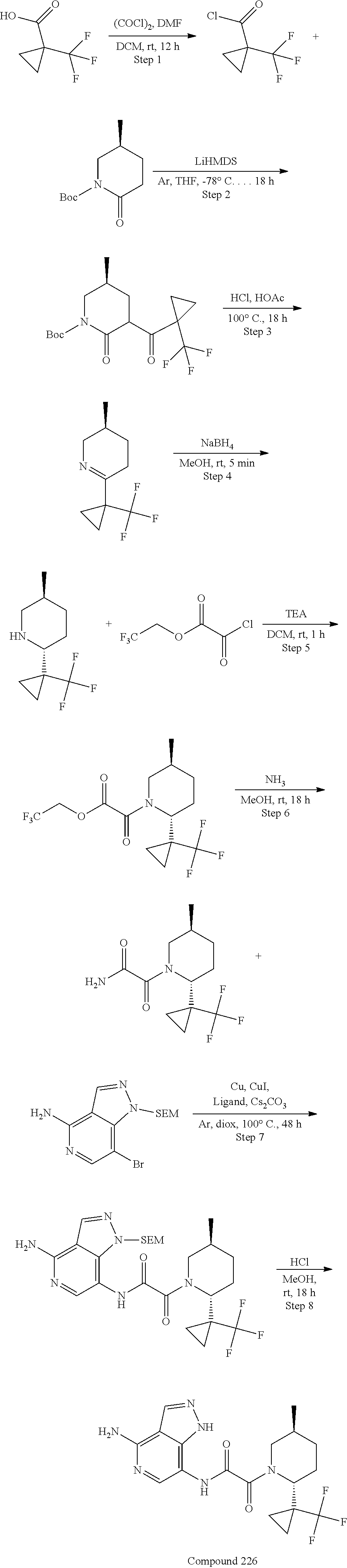

### Step 1: The Synthesis of 1-(Trifluoromethyl)cyclopropanecarbonyl chloride

1-(Trifluoromethyl)cyclopropanecarboxylic acid (5 g, 32.45 mmol) was dissolved in DCM (103.05 mL) and DMF (23.72 mg, 324.49 μmol, 25.13 μL) was added. Then Oxalyl chloride (4.53 g, 35.69 mmol, 3.11 mL) was added dropwise at 0° C. The mixture was allowed to stir at 25° C. for 12 hr. The reaction was washed with aqueous solution of NaHCO₃ and organic layer was collected, washed over Na₂SO₄ and evaporated to obtain 1-(trifluoromethyl)cyclopropanecarbonyl chloride (4.73 g, 27.42 mmol, 84.49% yield) as a colorless liquid.

¹H NMR (400 MHZ, CDCl₃) δ (ppm) 1.60 (m, 2H), 1.81 (m, H).

### Step 2: The Synthesis of tert-Butyl rac-(5S)-5-methyl-2-oxo-3-[1-(trifluoromethyl)cyclopropanecarbonyl]piperidine-1-carboxylate

At −78° C., Lithium bis(trimethylsilyl)amide (2.82 g, 16.88 mmol, 16.88 mL) was added dropwise to a solution of tert-butyl rac-(5S)-5-methyl-2-oxo-piperidine-1-carboxylate (3 g, 14.07 mmol) in THF (100 mL) under Ar flow. The resulting mixture was stirred at −78° C. for 1 hr. Then the solution of 1-(trifluoromethyl)cyclopropanecarbonyl chloride (2.91 g, 16.88 mmol) in THF was added at −78° C. to the previous mixture. After 30 min the solution was allowed warm to rt and stirred overnight. The reaction mixture was quenched with NaHSO₄ (10% solution) and extracted with DCM (3*50 mL). Organic layers was washed with water, dried over Na₂SO₄. DCM was evaporated to give tert-butyl rac-(5S)-5-methyl-2-oxo-3-[1-(trifluoromethyl)cyclopropanecarbonyl]piperidine-1-carboxylate (4.6 g, 13.17 mmol, 93.61% yield) as a beige solid.

LCMS(ESI): [M−tBu]⁺ m/z: calcd 294.2; found 294.2; Rt=2.586 min.

### Step 3: The Synthesis of rac-(3S)-3-Methyl-6-[1-(trifluoromethyl)cyclopropyl]-2,3,4,5-tetrahydropyridine

A solution of tert-butyl rac-(5S)-5-methyl-2-oxo-3-[1-(trifluoromethyl)cyclopropanecarbonyl]piperidine-1-carboxylate (4.6 g, 13.17 mmol) in AcOH (5 mL) and 6N HCl (5 mL) was heated at 100° C. for 48 hr. The reaction mixture was warmed to rt and the basified to pH 10 by 15% NaOH and then was extracted with DCM (3×50 mL). Organic phase was dried over Na₂SO₄, filtered and evaporated to obtain rac-(3S)-3-methyl-6-[1-(trifluoromethyl)cyclopropyl]-2,3,4,5-tetrahydropyridine (3.6 g, crude) as a brown oil.

¹H NMR (500 MHz, CDCl₃) δ (ppm) 0.91 (d, 3H), 1.22 (m, 5H), 1.58 (m, 1H), 1.76 (m, 1H), 2.32 (m, 1H), 2.49 (m, 1H), 3.01 (m, 1H), 3.73 (m, 1H).

### Step 4: The Synthesis of rac-(2R,5S)-5-Methyl-2-[1-(trifluoromethyl)cyclopropyl]piperidine

To an ice-cold solution of rac-(3S)-3-methyl-6-[1-(trifluoromethyl)cyclopropyl]-2,3,4,5-tetrahydropyridine (3.6 g, 12.28 mmol) in MeOH (14.60 mL) was added Sodium Borohydride (464.57 mg, 12.28 mmol, 432.56 μL) in one portion and the resulting mixture was stirred for 5 min. The reaction mixture was evaporated, partitioned in mixture of DCM/water. Water was extracted with DCM (50 ml). Combined organics were dried over Na₂SO₄ and evaporated to give a residue that was used in the next step without further purification. rac-(2R,5S)-5-Methyl-2-[1-(trifluoromethyl)cyclopropyl]piperidine (2.2 g, crude) was obtained as a yellow oil.

### Step 5: The Synthesis of 2,2,2-Trifluoroethyl 2-oxo-2-[rac-(2R,5S)-5-methyl-2-[1-(trifluoromethyl)cyclopropyl]-1-piperidyl]acetate

To a solution of rac-(2R,5S)-5-methyl-2-[1-(trifluoromethyl)cyclopropyl]piperidine (0.6 g, 2.90 mmol) and TEA (351.57 mg, 3.47 mmol, 484.25 μL) in DCM (25.32 mL) was added 2,2,2-trifluoroethyl 2-chloro-2-oxo-acetate (551.56 mg, 2.90 mmol) at rt. After stirring at rt for 1 hr the resulting mixture was washed with water, dried and evaporated to dryness to give 2,2,2-trifluoroethyl 2-oxo-2-[rac-(2R,5S)-5-methyl-2-[1-(trifluoromethyl)cyclopropyl]-1-piperidyl]acetate (1 g, 2.77 mmol, 95.60% yield) as a pale-yellow gum and was used in the next step without further purification.

LCMS(ESI): [M+H]⁺ m/z: calcd 362.2; found 362.2; Rt=3.701 min.

### Step 6: The Synthesis of 2-Oxo-2-[rac-(2R,5S)-5-methyl-2-[1-(trifluoromethyl)cyclopropyl]-1-piperidyl]acetamide

Ammonia (47.14 mg, 2.77 mmol) was bubbled through a solution of 2,2,2-trifluoroethyl 2-oxo-2-[rac-(2R,5S)-5-methyl-2-[1-(trifluoromethyl)cyclopropyl]-1-piperidyl]acetate (1 g, 2.77 mmol) in MeOH (50 mL) at rt. After stirring for 18 hr, the reaction mixture was evaporated to dryness to give a residue which was purified by CC (iNTERCHIM; 40 g SiO₂, CHCl₃/ACN with ACN from 0˜20%, flow rate=40 mL/min, Rv=7.5-10 CV) to give 2-oxo-2-[rac-(2R,5S)-5-methyl-2-[1-(trifluoromethyl)cyclopropyl]-1-piperidyl]acetamide (0.38 g, 1.37 mmol, 49.34% yield) as a yellow gum.

LCMS(ESI): [M+H]⁺ m/z: calcd 279.2; found 279.2; Rt=0.978 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-5-methyl-2-[1-(trifluoromethyl)cyclopropyl]-1-piperidyl]acetamide

2-Oxo-2-[rac-(2R,5S)-5-methyl-2-[1-(trifluoromethyl)cyclopropyl]-1-piperidyl]acetamide (0.15 g, 539.04 μmol), m7-bromo-1-(2-trimethylsilylethoxymethyl)pyrazolo[4,3-c]pyridin-4-amine (185.05 mg, 539.04 μmol), Copper (I) iodide (51.33 mg, 269.52 μmol, 9.13 μL), Cesium carbonate (351.26 mg, 1.08 mmol) and (1R,2R)—N1,N2-dimethylcyclohexane-1,2-diamine (76.67 mg, 539.04 μmol) were mixed in dioxane (5.00 mL) under argon, and then stirred for 72 h at 100° C. for 48 hr in vial. The reaction mixture was filtered and concentrated in vacuo. The residue was combined with another batch and purified by HPLC (45-65% 2-6 min; flow 30 mL/min water-MeCN+NH₃ (loading pump 4 mL/min MeCN); column xbridge C18 19*100 mm (L)). N-[4-Amino-1-(2-trimethylsilylethoxymethyl)pyrazolo[4,3-c]pyridin-7-yl]-2-oxo-2-[rac-(2R,5S)-5-methyl-2-[1-(trifluoromethyl)cyclopropyl]-1-piperidyl]acetamide (172.8 mg, 319.61 μmol, 59.29% yield) was obtained as a brown solid.

LCMS(ESI): [M+H]⁺ m/z: calcd 541.2; found 541.2; Rt=3.567 min.

### Step 8: The Synthesis of N-(4-Amino-1H-pyrazolo[4,3-c]pyridin-7-yl)-2-oxo-2-[rac-(2R,5S)-5-methyl-2-[1-(trifluoromethyl)cyclopropyl]-1-piperidyl]acetamide (Compound 226)

To a solution of N-[4-amino-1-(2-trimethylsilylethoxymethyl)pyrazolo[4,3-c]pyridin-7-yl]-2-oxo-2-[rac-(2R,5S)-5-methyl-2-[1-(trifluoromethyl)cyclopropyl]-1-piperidyl]acetamide (172.8 mg, 230.12 μmol) in MeOH (2 mL) was added Hydrogen chloride solution 4.0M in dioxane (1.60 g, 43.88 mmol, 2 mL) at 21° C. The resulting mixture was left to stir for 18 hr. The resulting mixture was evaporated to dryness and subjected to HPLC (20-40% 2-7 min; flow 30 mL/min water-ACN+NH₃ (loading pump 4 mL/min ACN); column XBRIDGE C18 19*100 mm (L)). N-(4-Amino-1H-pyrazolo[4,3-c]pyridin-7-yl)-2-oxo-2-[rac-(2R,5S)-5-methyl-2-[1-(trifluoromethyl)cyclopropyl]-1-piperidyl]acetamide (64 mg, 155.95 μmol, 67.77% yield) was obtained as a beige solid.

¹H NMR (600 MHz, dmso) δ 0.83-0.98 (m, 7H), 1.05-1.18 (m, 1H), 1.59-1.79 (m, 1H), 1.83-2.05 (m, 3H), 3.33-3.41 (m, 1H), 3.68-4.18 (m, 1H), 4.38-4.92 (m, 1H), 6.59-6.83 (m, 2H), 7.43-7.81 (m, 1H), 8.05-8.20 (m, 1H), 9.42-10.60 (m, 1H), 12.45-13.46 (m, 1H).

LCMS(ESI): [M+H]⁺ m/z: calcd 411.2; found 411.2; Rt=1.150 min.

### Example 203. N-(4-Amino-2H-pyrazolo[3,4-c]pyridin-7-yl)-2-oxo-2-[rac-(2S,5S)-2,5-diethyl-1-piperidyl]acetamide (Compound 233) and N-(4-Amino-2H-pyrazolo[3,4-c]pyridin-7-yl)-2-oxo-2-[rac-(2S,5R)-2,5-diethyl-1-piperidyl]acetamide (Compound 227)

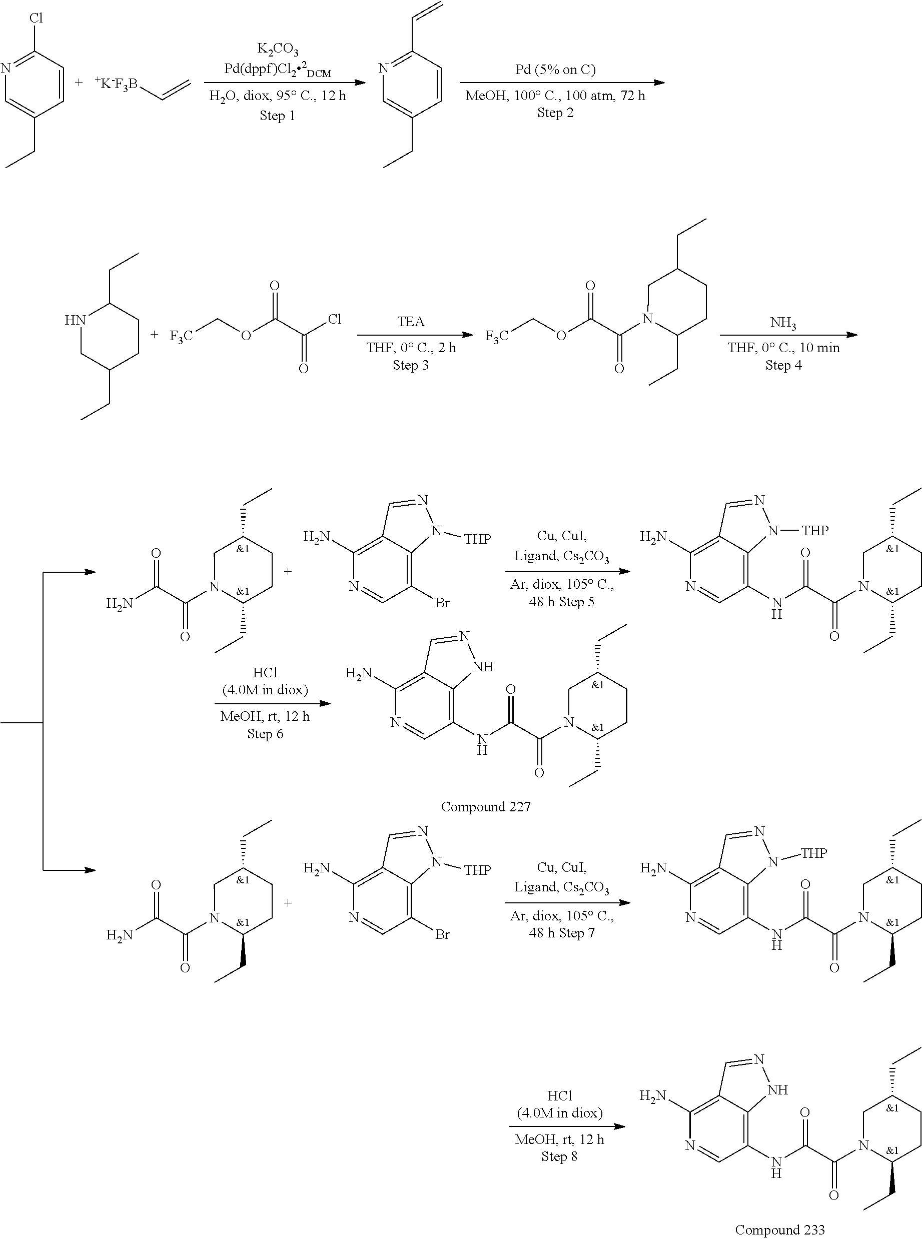

### Step 1: The Synthesis of 5-Ethyl-2-vinyl-pyridine

To a solution of 2-chloro-5-ethyl-pyridine (4 g, 28.25 mmol), potassium ethenyltrifluoroboranuide (4.92 g, 36.72 mmol) and potassium carbonate (19.52 g, 141.25 mmol, 8.52 mL) in Dioxane (150 mL) and Water (50 mL), Pd(dppf)Cl₂*CH₂Cl₂ (1.15 g, 1.41 mmol) was added. The resulting mixture was stirred at 95° C. for 12 hr, cooled, organic layer separated, the solvent was evaporated in vacuo, the residue was dissolved in MTBE, washed with water dried over Na₂SO₄ and evaporated to give crude product (3.5 g). These substances were combined with another batch and dissolved in hexane. Some impurities were filtered and the solvent was evaporated to give 5-ethyl-2-vinyl-pyridine (3.3 g, 24.78 mmol, 87.71% yield).

LCMS(ESI): [M+H]⁺ m/z: calcd 134.0; found 134.0; Rt=0.467 min.

### Step 2: The Synthesis of 2,5-Diethylpiperidine

A mixture of 5-ethyl-2-vinyl-pyridine (3.3 g, 24.78 mmol), Palladium on carbon 5% (3.06 g, 24.78 mmol) in Methanol (100 mL) was stirred at 100° C. under pressure 100 atm of hydrogen for 72 hr. The reaction mixture was cooled, the catalyst was filtered off and the solvent was removed in vacuo to give 2,5-diethylpiperidine (2.8 g, 19.82 mmol, 80.01% yield) (mixture of diastereomers).

LCMS(ESI): [M+H]⁺ m/z: calcd 142.2; found 142.2; Rt=0.433 min.

### Step 3: The Synthesis of 2,2,2-Trifluoroethyl 2-(2,5-diethyl-1-piperidyl)-2-oxo-acetate

To a solution of 2,5-diethylpiperidine (2.8 g, 19.82 mmol) and Triethylamine (4.01 g, 39.65 mmol, 5.53 mL) in THF (70 mL), 2,2,2-trifluoroethyl 2-chloro-2-oxo-acetate (7.55 g, 39.65 mmol) was added portionwise at 0° C. The resulting mixture was stirred for 2 hr. LCMS showed full conversion of SM. The reaction mixture was directly used for the next step.

LCMS(ESI): [M+H]⁺ m/z: calcd 296.2; found 296.2; Rt=1.086 min.

### Step 4: The Synthesis of 2-Oxo-2-[rac-(2S,5S)-2,5-diethyl-1-piperidyl]acetamide and 2-Oxo-2-[rac-(2S,5R)-2,5-diethyl-1-piperidyl]acetamide

Through a solution of 2,2,2-trifluoroethyl 2-(2,5-diethyl-1-piperidyl)-2-oxo-acetate (5.5 g, 18.63 mmol) in THF (50 mL), ammonia was bubbled during 10 min at 0° C. The formed precipitate was filtered off, washed with THF (30 ml) and the solvent was evaporated in vacuo to give crude product (6 g), which was purified by gradient chromatography (MTBE —CHCl₃) to obtain 2.5 g mixture of diastereomers. This mixture (1 g) was separated by HPLC (Device (Mobile Phase, Column): SYSTEM 10-10-45% 0-1-5 min H₂O/ACN, flow: 30 mL/min (loading pump 4 mL/min acetonitrile; column: Chromatorex 18 SMB100-5T 100×19 mm 5 um) to give solid 2-oxo-2-[rac-(2S,5S)-2,5-diethyl-1-piperidyl]acetamide (703 mg, 3.31 mmol, 35.56% yield) and oil 2-oxo-2-[rac-(2S,5R)-2,5-diethyl-1-piperidyl]acetamide (284 mg, 1.34 mmol, 14.37% yield).

### 2-Oxo-2-[rac-(2S,5S)-2,5-diethyl-1-piperidyl]acetamide

LCMS(ESI): [M+H]⁺ m/z: calcd 213.2; found 213.2; Rt=2.321 min.

### 2-Oxo-2-[rac-(2S,5R)-2,5-diethyl-1-piperidyl]acetamide

LCMS(ESI): [M+H]⁺ m/z: calcd 231.2; found 213.2; Rt=2.404 min.

### Step 5: The Synthesis of N-(4-Amino-2-tetrahydropyran-2-yl-pyrazolo[3,4-c]pyridin-7-yl)-2-oxo-2-[rac-(2S,5R)-2,5-diethyl-1-piperidyl]acetamide

To a mixture of 2-oxo-2-[rac-(2S,5R)-2,5-diethyl-1-piperidyl]acetamide (0.1 g, 471.06 μmol), 7-bromo-2-tetrahydropyran-2-yl-pyrazolo[3,4-c]pyridin-4-amine (223.96 mg, 753.69 μmol), Copper (5.99 mg, 94.21 μmol), Copper (I) iodide (89.71 mg, 471.06 μmol, 15.96 μL), Cesium carbonate (306.96 mg, 942.12 μmol) and rac-(1R,2R)—N1,N2-dimethylcyclohexane-1,2-diamine (100.51 mg, 706.59 μmol), Dioxane was added. The resulting mixture was evacuated, refiled with Argon, heated at 100° C. for 18 hr and cooled. The inorganic precipitate was filtered and washed with DCM (20 mL) to give N-(4-amino-2-tetrahydropyran-2-yl-pyrazolo[3,4-c]pyridin-7-yl)-2-oxo-2-[rac-(2S,5R)-2,5-diethyl-1-piperidyl]acetamide (0.2 g, 466.72 μmol, 99.08% yield).

LCMS(ESI): [M+H]⁺ m/z: calcd 429.2; found 429.2; Rt=0.873 min.

### Step 6: The Synthesis of N-(4-Amino-2H-pyrazolo[3,4-c]pyridin-7-yl)-2-oxo-2-[rac-(2S,5R)-2,5-diethyl-1-piperidyl]acetamide (Compound 227)

To a solution of N-(4-amino-2-tetrahydropyran-2-yl-pyrazolo[3,4-c]pyridin-7-yl)-2-oxo-2-[rac-(2S,5R)-2,5-diethyl-1-piperidyl]acetamide (0.2 g, 466.72 μmol) in MeOH (4 mL), Hydrogen chloride solution 4.0M in dioxane (2 g, 54.85 mmol, 2.50 mL) was added. The resulting mixture was stirred at 25° C. for 6 hr and evaporated in vacuo. The residue was purified by HPLC (Device (Mobile Phase, Column): SYSTEM 5-55% 0-5 min H₂O/ACN/0.1% FA, flow: 30 mL/min (loading pump 4 mL/min H₂O); column: Chromatorex 18 SMB100-5T 100×19 mm 5 um) to obtain N-(4-amino-2H-pyrazolo[3,4-c]pyridin-7-yl)-2-oxo-2-[rac-(2S,5R)-2,5-diethyl-1-piperidyl]acetamide (43 mg, 110.13 μmol, 23.60% yield, HCO2H).

¹H NMR (600 MHz, dmso) δ 0.77-0.94 (m, 6H), 0.99-1.42 (m, 5H), 1.49-1.79 (m, 5H), 3.75-4.01 (m, 1H), 4.25-4.47 (m, 1H), 6.57-6.87 (m, 2H), 7.35-7.78 (m, 1H), 8.12 (s, 1H), 8.19 (d, 1H), 9.33-10.52 (m, 1H), 12.12-13.49 (m, 1H).

LCMS(ESI): [M+H]⁺ m/z: calcd 345.2; found 345.2; Rt=2.199 min.

### Step 7: The Synthesis of N-(4-Amino-2-tetrahydropyran-2-yl-pyrazolo[3,4-c]pyridin-7-yl)-2-oxo-2-[rac-(2S,5S)-2,5-diethyl-1-piperidyl]acetamide

To a mixture of 2-oxo-2-[rac-(2S,5S)-2,5-diethyl-1-piperidyl]acetamide (0.2 g, 942.12 μmol), 7-bromo-2-tetrahydropyran-2-yl-pyrazolo[3,4-c]pyridin-4-amine (447.92 mg, 1.51 mmol), Copper (11.97 mg, 188.42 μmol), Copper (I) iodide (179.43 mg, 942.12 μmol, 31.93 μL), Cesium carbonate (613.92 mg, 1.88 mmol) and rac-(1R,2R)—N1,N2-dimethylcyclohexane-1,2-diamine (201.01 mg, 1.41 mmol), Dioxane (5 mL) was added. The resulting mixture was evacuated, refiled with Argon, heated at 100° C. for 18 hr and cooled. The inorganic precipitate was filtered and washed with DCM (20 mL) to give N-(4-amino-2-tetrahydropyran-2-yl-pyrazolo[3,4-c]pyridin-7-yl)-2-oxo-2-[rac-(2S,5S)-2,5-diethyl-1-piperidyl]acetamide (0.4 g, 933.43 μmol, 99.08% yield).

LCMS(ESI): [M+H]⁺ m/z: calcd 429.2; found 429.2; Rt=0.858 min.

### Step 8: The Synthesis of N-(4-Amino-2H-pyrazolo[3,4-c]pyridin-7-yl)-2-oxo-2-[rac-(2S,5S)-2,5-diethyl-1-piperidyl]acetamide (Compound 233)

To a solution of N-(4-amino-2-tetrahydropyran-2-yl-pyrazolo[3,4-c]pyridin-7-yl)-2-oxo-2-[rac-(2S,5S)-2,5-diethyl-1-piperidyl]acetamide (0.4 g, 933.43 μmol) in MeOH (5 mL), Hydrogen chloride solution 4.0M in dioxane (3 g, 82.28 mmol, 3.75 mL) was added. The resulting mixture was stirred at 25° C. for 12 hr and evaporated in vacuo. The residue was purified by HPLC (Device (Mobile Phase, Column): SYSTEM 5-5-35 0-1-5 min H₂O/ACN/0.1% FA, flow: 30 mL/min (loading pump 4 mL/min acetonitrile); column: Chromatorex 18 SMB100-5T 100×19 mm 5 um) to obtain N-(4-amino-2H-pyrazolo[3,4-c]pyridin-7-yl)-2-oxo-2-[rac-(2S,5S)-2,5-diethyl-1-piperidyl]acetamide (109 mg, 279.18 μmol, 29.91% yield, HCO₂H).

¹H NMR (600 MHz, dmso) δ 0.44-0.93 (m, 6H), 1.20-1.93 (m, 9H), 2.86-3.34 (m, 1H), 3.68-4.45 (m, 2H), 6.49-6.96 (m, 2H), 7.34-7.79 (m, 1H), 8.04-8.33 (m, 2H), 9.36-10.44 (m, 1H), 12.43-13.51 (m, 1H).

LCMS(ESI): [M+H]⁺ m/z: calcd 345.2; found 345.2; Rt=2.113 min.

### Example 204. N-(4-Amino-1H-pyrazolo[4,3-c]pyridin-7-yl)-2-oxo-2-[rac-(2R,5S)-5-methyl-2-(1-methylcyclopropyl)-1-piperidyl]acetamide (Compound 228)

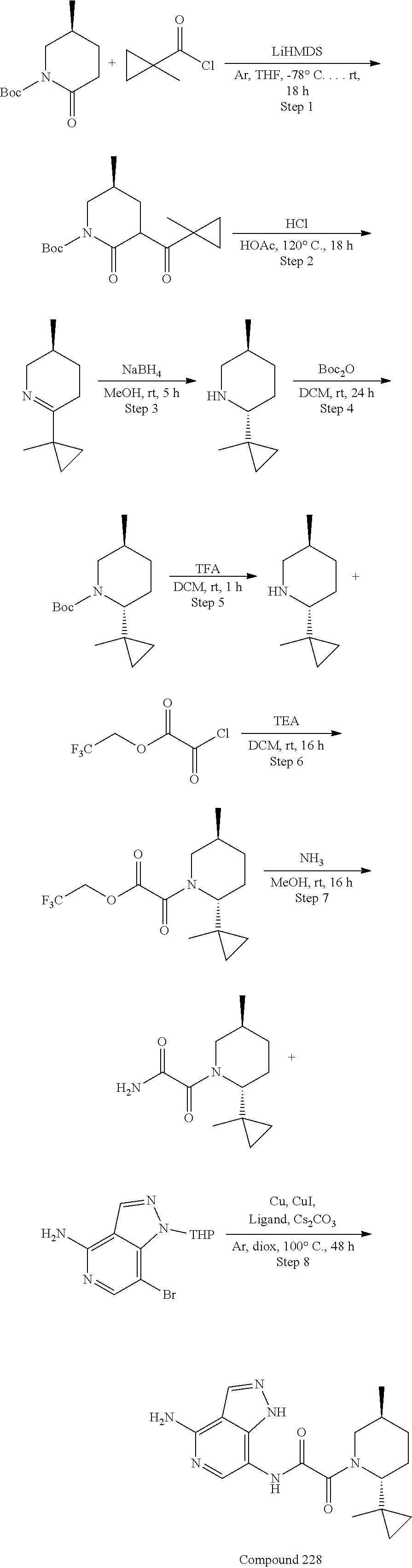

### Step 1: The Synthesis of tert-Butyl rac-(5S)-5-methyl-3-(1-methylcyclopropanecarbonyl)-2-oxo-piperidine-1-carboxylate

tert-Butyl rac-(5S)-5-methyl-2-oxo-piperidine-1-carboxylate (3.5 g, 16.41 mmol) was dissolved in THF (40 mL) and the resulting solution was cooled to −78° C. under an argon atmosphere. Lithium bis(trimethylsilyl)amide (16.48 g, 19.69 mmol, 18.31 mL, 20% purity) was added dropwise at −78° C. and the resulting mixture was stirred at −78° C. for 1 hr. A solution of 1-methylcyclopropanecarbonyl chloride (2.33 g, 19.69 mmol) in THF (7 mL) was added dropwise and the resulting mixture was stirred at −78° C. for 30 min. The reaction mixture was allowed to warm to room temperature. Aq. NaHSO₄ (7 g in 70 mL of water) was added dropwise and the resulting mixture was stirred for 15 min. The previous mixture was extracted with EtOAc (2*100 mL) and combined organic layers were washed with brine (70 mL), dried over Na₂SO₄, filtered, and concentrated in vacuo to obtain tert-butyl rac-(5S)-5-methyl-3-(1-methylcyclopropanecarbonyl)-2-oxo-piperidine-1-carboxylate (5.33 g, crude) which was used in the next step without purification.

LCMS(ESI): [M−tBu]⁺ m/z: calcd 240.2; found 240.2; Rt=1.415 min.

### Step 2: The Synthesis of rac-(3S)-3-Methyl-6-(1-methylcyclopropyl)-2,3,4,5-tetrahydropyridine

tert-Butyl rac-(5S)-5-methyl-3-(1-methylcyclopropanecarbonyl)-2-oxo-piperidine-1-carboxylate (5.33 g, 18.04 mmol) was dissolved in AcOH (15 mL) and 6N HCl (15 mL) was added thereto. The resulting mixture was heated at 120° C. overnight. The reaction mixture was cooled and basified with K₂CO₃. The resulting mixture was diluted with water (50 mL) and extracted with DCM (2*50 mL). Combined organic layers were dried over Na₂SO₄, filtered, and concentrated in vacuo to obtain rac-(3S)-3-methyl-6-(1-methylcyclopropyl)-2,3,4,5-tetrahydropyridine (2.05 g, 13.55 mmol, 75.11% yield) which was used in the next step without purification.

LCMS(ESI): [M+H]⁺ m/z: calcd 152.0; found 152.0; Rt=0.444 min.

### Step 3: The Synthesis of rac-(2R,5S)-5-Methyl-2-(1-methylcyclopropyl)piperidine

rac-(3S)-3-Methyl-6-(1-methylcyclopropyl)-2,3,4,5-tetrahydropyridine (2.05 g, 13.55 mmol) was dissolved in MeOH (200 mL) and the resulting solution was cooled to 0° C. Sodium Borohydride (512.78 mg, 13.55 mmol, 477.45 μL) was added in one portion and the resulting mixture was stirred for 5 min. The reaction mixture was concentrated in vacuo and water (50 mL) was added to the residue. The resulting mixture was extracted with DCM (2*50 mL) and combined organic layers were dried over Na₂SO₄, filtered, and concentrated in vacuo to obtain rac-(2R,5S)-5-methyl-2-(1-methylcyclopropyl)piperidine (1.82 g, 11.87 mmol, 87.61% yield) which was used in the next step without purification.

LCMS(ESI): [M+H]⁺ m/z: calcd 154.2; found 154.2; Rt=0.379 min.

### Step 4: The Synthesis of tert-Butyl rac-(2R,5S)-5-methyl-2-(1-methylcyclopropyl)piperidine-1-carboxylate

rac-(2R,5S)-5-Methyl-2-(1-methylcyclopropyl)piperidine (1.82 g, 11.87 mmol) was dissolved in DCM (50 mL) and Di-tert-butyl dicarbonate (5.18 g, 23.75 mmol, 5.45 mL) was added dropwise. The resulting mixture was stirred over the weekend. The reaction mixture was concentrated in vacuo and the residue was purified by FCC (from 4:1 hexane-THF to 3:2 hexane-THF) to obtain tert-butyl N-[rac-(2S)-5-hydroxy-2-methyl-5-(1-methylcyclopropyl)pentyl]carbamate (1.74 g, 6.42 mmol, 54.05% yield) (side product) and crude product which was re-purified by FCC (from 5:1 hexane-MTBE to 2:1 hexane MTBE) to obtain tert-butyl rac-(2R,5S)-5-methyl-2-(1-methylcyclopropyl)piperidine-1-carboxylate (337 mg, 1.33 mmol, 11.20% yield) and tert-butyl rac-(2R,5S)-5-methyl-2-(1-methylcyclopropyl)piperidine-1-carboxylate (402 mg, 1.59 mmol, 13.36% yield).

LCMS(ESI): [M−tBu]⁺ m/z: calcd 198.0; found 198.0; Rt=1.633 min.

### Step 5: The Synthesis of rac-(2R,5S)-5-Methyl-2-(1-methylcyclopropyl)piperidine

tert-Butyl rac-(2R,5S)-5-methyl-2-(1-methylcyclopropyl)piperidine-1-carboxylate (337 mg, 1.33 mmol) was dissolved in DCM (2 mL) and TFA (2 mL) was added. The resulting solution was stirred for 1 hr. The reaction mixture was carefully poured into aq. K₂CO₃ solution (5 g in 15 ml of water) and the resulting mixture was extracted with DCM (2*20 mL). Combined organic layers were dried over Na₂SO₄, filtered, and concentrated in vacuo to obtain rac-(2R,5S)-5-methyl-2-(1-methylcyclopropyl)piperidine (140 mg, 913.46 μmol, 68.68% yield).

### Step 6: The Synthesis of 2,2,2-Trifluoroethyl 2-oxo-2-[rac-(2R,5S)-5-methyl-2-(1-methylcyclopropyl)-1-piperidyl]acetate

rac-(2R,5S)-5-Methyl-2-(1-methylcyclopropyl)piperidine (140 mg, 913.46 μmol) and Triethylamine (101.68 mg, 1.00 mmol, 140.24 μL) were dissolved in DCM (5 mL) and the resulting solution was cooled to −5° C. in an ice/methanol bath. 2,2,2-trifluoroethyl 2-chloro-2-oxo-acetate (182.72 mg, 959.13 μmol) was added dropwise and the resulting mixture was allowed to warm to room temperature and stirred overnight. Water (5 mL) was added and an organic layer was separated. The aqueous layer was extracted with DCM (2*10 mL) and combined organic layers were dried over Na₂SO₄, filtered and concentrated in vacuo to obtain 2,2,2-trifluoroethyl 2-oxo-2-[rac-(2R,5S)-5-methyl-2-(1-methylcyclopropyl)-1-piperidyl]acetate (200 mg, 650.81 μmol, 71.25% yield).

LCMS(ESI): [M+H]⁺ m/z: calcd 308.2; found 308.2; Rt=1.541 min.

### Step 7: The Synthesis of 2-Oxo-2-[rac-(2R,5S)-5-methyl-2-(1-methylcyclopropyl)-1-piperidyl]acetamide

2,2,2-Trifluoroethyl 2-oxo-2-[rac-(2R,5S)-5-methyl-2-(1-methylcyclopropyl)-1-piperidyl]acetate (200 mg, 650.81 μmol) was dissolved in MeOH (5 mL) and NH₃/MeOH (7 mL) was added thereto. The resulting mixture was stirred overnight. The reaction mixture was concentrated in vacuo. The residue was combined with the residue from another batch and purified by HPLC (0-2-9 min, 28-35-55% H₂O/MeOH/0.1NH₄OH, flow 30 mL/min ((loading pump 4 mL MeOH); column: XBridge BEH C18 100*19 mm, 5 microM) to obtain 2-oxo-2-[rac-(2R,5S)-5-methyl-2-(1-methylcyclopropyl)-1-piperidyl]acetamide (36.7 mg, 163.62 μmol, 25.14% yield).

LCMS(ESI): [M+H]⁺ m/z: calcd 225.2; found 225.2; Rt=0.937 min.

### Step 8: The Synthesis of N-(4-Amino-1H-pyrazolo[4,3-c]pyridin-7-yl)-2-oxo-2-[rac-(2R,5S)-5-methyl-2-(1-methylcyclopropyl)-1-piperidyl]acetamide (Compound 228)

2-Oxo-2-[rac-(2R,5S)-5-methyl-2-(1-methylcyclopropyl)-1-piperidyl]acetamide (36.7 mg, 163.62 μmol), 7-bromo-2-tetrahydropyran-2-yl-pyrazolo[4,3-c]pyridin-4-amine (53.48 mg, 179.98 μmol), Copper (519.91 μg, 8.18 μmol), Copper (I) iodide (15.58 mg, 81.81 μmol, 2.77 μL), rac-(1R,2R)—N1,N2-dimethylcyclohexane-1,2-diamine (17.46 mg, 122.72 μmol) and Cesium carbonate (106.62 mg, 327.24 μmol) were mixed in Dioxane (1.5 mL). The resulting mixture was purged with argon for 5 min. The reaction mixture was heated at 100° C. for 48 hr. The reaction mixture was cooled and filtered. The filter cake was rinsed with MeOH (5 mL) and the filtrate was concentrated in vacuo. Formic acid (2 mL) was added to the residue and the resulting mixture was stirred overnight. The resulting mixture was submitted to HPLC and purified (0-90%, 0-2-10 min, H₂O/MeOH/0.1FA, flow 30 mL/min ((loading pump 4 mL MeCN); column: Chromatorex C18 SMB100-5T 100*19 mm, 5 microM) to obtain N-(4-amino-1H-pyrazolo[4,3-c]pyridin-7-yl)-2-oxo-2-[rac-(2R,5S)-5-methyl-2-(1-methylcyclopropyl)-1-piperidyl]acetamide (10.4 mg, 25.84 μmol, 15.79% yield, HCOOH).

¹H NMR (600 MHz, dmso) δ 0.13-0.75 (m, 5H), 0.79-1.08 (m, 8H), 1.42-1.71 (m, 2H), 1.84-2.02 (m, 2H), 3.92-4.03 (m, 1H), 6.62-6.72 (m, 2H), 7.43-8.17 (m, 2H), 9.37-10.46 (m, 1H), 12.49-13.36 (m, 1H).

LCMS(ESI): [M+H]⁺ m/z: calcd 357.2; found 357.2; Rt=0.996 min.

### Example 205. N-(4-Amino-1H-pyrazolo[4,3-c]pyridin-7-yl)-2-oxo-2-[rac-(2R,5S)-2-(4-hydroxyphenyl)-5-methyl-1-piperidyl]acetamide (Compound 229)

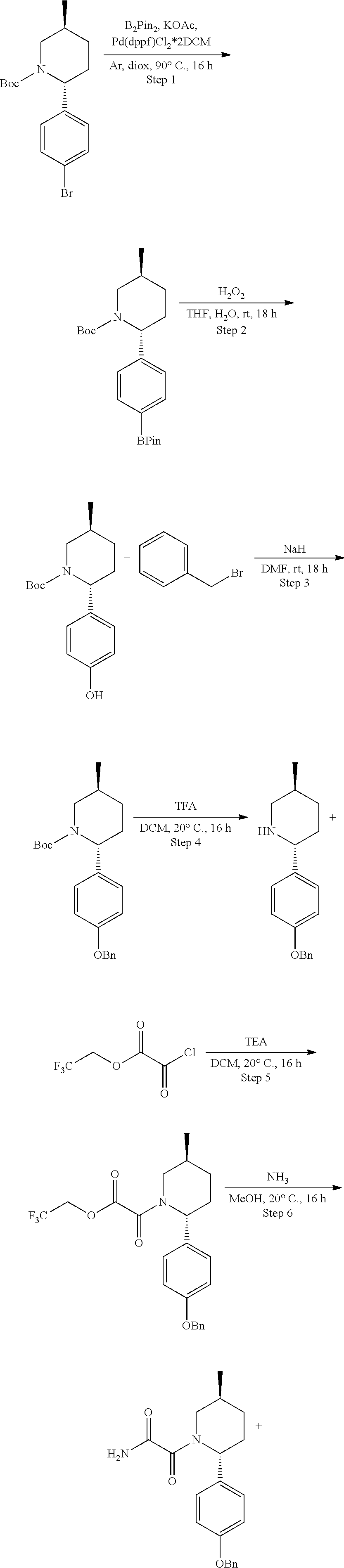

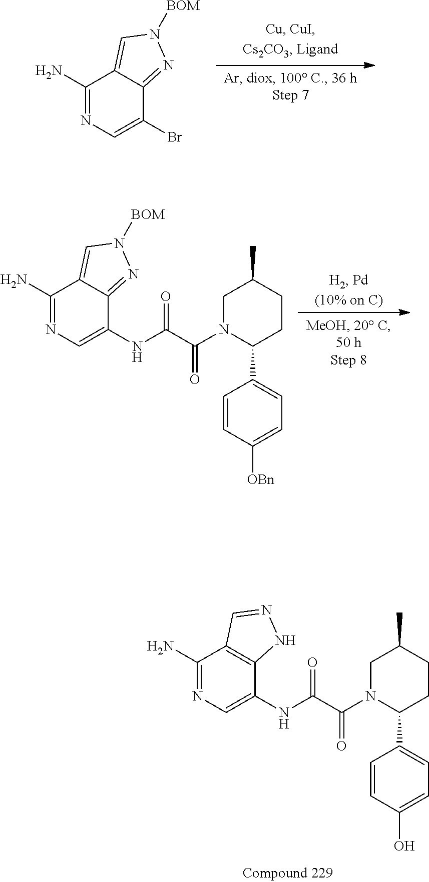

### Step 1: The Synthesis of tert-Butyl rac-(2R,5S)-5-methyl-2-[4-(4,4,5,5-tetramethyl-1,3,2-dioxaborolan-2-yl)phenyl]piperidine-1-carboxylate

To a solution of tert-butyl rac-(2R,5S)-2-(4-bromophenyl)-5-methyl-piperidine-1-5 carboxylate (1 g, 2.82 mmol), Potassium Acetate (637.13 mg, 6.49 mmol, 405.81 μL) and 4,4,5,5-tetramethyl-2-(4,4,5,5-tetramethyl-1,3,2-dioxaborolan-2-yl)-1,3,2-dioxaborolane (788.45 mg, 3.10 mmol) in dioxane (15 mL) was added Pd(dppf)₂Cl₂ DCM (138.30 mg, 169.36 μmol) under argon. The reaction mixture was stirred at 90° C. for 16 hr. Then the reaction mixture was concentrated, dissolved in 10 mL of ethyl acetate and washed with water. The organic layer was dried with sodium sulfate and evaporated under reduced pressure. The residue was purified by FCC (Interchim 80 g SiO₂, MTBE-MeOH from 0˜100%, flow rate=70 mL/min, cv=1.3) to give tert-butyl rac-(2R,5S)-5-methyl-2-[4-(4,4,5,5-tetramethyl-1,3,2-dioxaborolan-2-yl)phenyl]piperidine-1-carboxylate.

### Step 2: The Synthesis of tert-Butyl rac-(2R,5S)-2-(4-hydroxyphenyl)-5-methyl-piperidine-1-carboxylate

To a solution of tert-butyl rac-(2R,5S)-5-methyl-2-[4-(4,4,5,5-tetramethyl-1,3,2-dioxaborolan-2-yl)phenyl]piperidine-1-carboxylate (1.1 g, 2.74 mmol) in mixture of H₂O (10 mL) and THF (10 mL) Hydrogen peroxide 35% (699.10 mg, 20.56 mmol, 629.82 μL) was added and stirred overnight. The reaction mixture was diluted with water (40 mL) and MTBE (40 mL), and treated with sodium thiosulfate, then organic phase was washed with water, dried over Na₂SO₄ and concentrated in vacuo to give tert-butyl rac-(2R,5S)-2-(4-hydroxyphenyl)-5-methyl-piperidine-1-carboxylate (0.85 g, 2.92 mmol, 106.43% yield).

### Step 3: The Synthesis of tert-Butyl rac-(2R,5S)-2-(4-benzyloxyphenyl)-5-methyl-piperidine-1-carboxylate

tert-Butyl rac-(2R,5S)-2-(4-hydroxyphenyl)-5-methyl-piperidine-1-carboxylate (0.85 g, 2.92 mmol) was dissolved in DMF (10 mL) and Sodium Hydride (in oil dispersion) 60% dispersion in mineral oil (128.35 mg, 3.21 mmol, 60% purity) was added in one portion. The resulting mixture was stirred for 15 min and bromomethylbenzene (548.82 mg, 3.21 mmol, 381.65 μL) was added. The resulting mixture was stirred overnight. The reaction mixture was diluted with aq. NH₄Cl solution (10 mL) and the resulting mixture was extracted with EtOAc (2*15 mL). Combined organic layers were washed with water (3*10 mL), brine (10 mL), dried over Na₂SO₄, filtered, and concentrated in vacuo to obtain tert-butyl rac-(2R,5S)-2-(4-benzyloxyphenyl)-5-methyl-piperidine-1-carboxylate.

### Step 4: The Synthesis of rac-(2R,5S)-2-(4-Benzyloxyphenyl)-5-methyl-piperidine

Trifluoroacetic acid (2.98 g, 26.14 mmol, 2 mL) was added in one portion to a stirred solution of tert-butyl rac-(2R,5S)-2-(4-benzyloxyphenyl)-5-methyl-piperidine-1-carboxylate (0.96 g, 2.52 mmol) in dichloromethane (10 mL). The resulting solution was stirred at 20° C. for 16 hr, and then concentrated in vacuo. The residue was diluted with water (20 mL), then basified to pH 11-12 with 10% aqueous sodium carbonate solution and extracted with dichloromethane (2*20 mL). The combined organic extracts were dried over sodium sulfate and concentrated in vacuo to afford rac-(2R,5S)-2-(4-benzyloxyphenyl)-5-methyl-piperidine (690 mg, 2.45 mmol, 97.45% yield) which was directly used in the next step.

### Step 5: The Synthesis of 2,2,2-Trifluoroethyl 2-oxo-2-[rac-(2R,5S)-2-(4-benzyloxyphenyl)-5-methyl-1-piperidyl]acetate

2,2,2-Trifluoroethyl 2-chloro-2-oxo-acetate (511.62 mg, 2.69 mmol) was added dropwise to a solution of rac-(2R,5S)-2-(4-benzyloxyphenyl)-5-methyl-piperidine (687 mg, 2.44 mmol) and triethylamine (296.46 mg, 2.93 mmol, 408.35 μL) in DCM at 0° C. After addition was complete, cooling bath was removed and resulting mixture was allowed to warm up to 20° C. and stirred for 16 hr. Then, it was washed with water, dried over Na₂SO₄ and concentrated under reduced pressure, affording 2,2,2-trifluoroethyl 2-oxo-2-[rac-(2R,5S)-2-(4-benzyloxyphenyl)-5-methyl-1-piperidyl]acetate which was used in the next step without further purification.

### Step 6: The Synthesis of 2-Oxo-2-[rac-(2R,5S)-2-(4-benzyloxyphenyl)-5-methyl-1-piperidyl]acetamide

A solution of 2,2,2-trifluoroethyl 2-oxo-2-[rac-(2R,5S)-2-(4-benzyloxyphenyl)-5-methyl-1-piperidyl]acetate (1 g, 1.42 mmol) in Methanol/NH₃ (5N) (5 mL) was stirred at 20° C. for 16 hr. The solvent was evaporated and then submitted to reverse phase HPLC (28-35-50% H₂O-MeCN, 0-2-10 min, flow 30 mL/min ((loading pump 4 mL MeCN); column: Chromatorex C18 SMB100-5T 100*19 mm, 5 microM) to obtain 2-oxo-2-[rac-(2R,5S)-2-(4-benzyloxyphenyl)-5-methyl-1-piperidyl]acetamide (270 mg, 766.12 μmol, 53.81% yield).

LCMS(ESI): [M+H]⁺ m/z: calcd 353.2; found 353.2; Rt=1.382 min.

### Step 7: The Synthesis of N-[4-Amino-2-(benzyloxymethyl)pyrazolo[4,3-c]pyridin-7-yl]-2-oxo-2-[rac-(2R,5S)-2-(4-benzyloxyphenyl)-5-methyl-1-piperidyl]acetamide

2-Oxo-2-[rac-(2R,5S)-2-(4-benzyloxyphenyl)-5-methyl-1-piperidyl]acetamide (106 mg, 300.77 μmol), 2-(benzyloxymethyl)-7-bromo-pyrazolo[4,3-c]pyridin-4-amine (100.21 mg, 300.77 μmol), Copper (I) iodide (11.46 mg, 60.15 μmol, 2.04 μL), Cesium carbonate (196.00 mg, 601.54 μmol) and (1R,2R)—N1,N2-dimethylcyclohexane-1,2-diamine (51.34 mg, 360.93 μmol) were mixed in dioxane (5 mL) under argon, and then stirred overnight at 100° C. for 36 hr in vial. The reaction mixture was filtered, and the filtrate was concentrated in vacuo and then submitted to reverse phase HPLC (0-2-9 min 43-50-70% H₂O-MeCN, flow 30 mL/min (loading pump 4 mL MeCN), column: Chromatorex C18 SMB100-5T 100*19 mm 5 microM) to give N-[4-amino-2-(benzyloxymethyl)pyrazolo[4,3-c]pyridin-7-yl]-2-oxo-2-[rac-(2R,5S)-2-(4-benzyloxyphenyl)-5-methyl-1-piperidyl]acetamide (48 mg, 79.38 μmol, 26.39% yield).

LCMS(ESI): [M+H]⁺ m/z: calcd 605.2; found 605.4; Rt=1.298 min.

### Step 8: The Synthesis of N-(4-Amino-1H-pyrazolo[4,3-c]pyridin-7-yl)-2-oxo-2-[rac-(2R,5S)-2-(4-hydroxyphenyl)-5-methyl-1-piperidyl]acetamide (Compound 229)

The mixture of N-[4-amino-2-(benzyloxymethyl)pyrazolo[4,3-c]pyridin-7-yl]-2-oxo-2-[rac-(2R,5S)-2-(4-benzyloxyphenyl)-5-methyl-1-piperidyl]acetamide (33 mg, 54.57 μmol) and Palladium, 10% on carbon, Type 487, dry (5.81 mg, 54.57 μmol) in MeOH (3 mL) was subjected to Hydrogenation (1 atm) at 20° C. for 50 hr. Then the mixture was filtered and evaporated to dryness. The crude material was combined with another batch and submitted to HPLC (15 mM NH₄HCO₃-ACN 15-40% ACN 0.5-6 min, flow 5 mL/min. Chromatorex Phenyl 7.8*75 mm, 5 mkm) affording N-(4-amino-1H-pyrazolo[4,3-c]pyridin-7-yl)-2-oxo-2-[rac-(2R,5S)-2-(4-hydroxyphenyl)-5-methyl-1-piperidyl]acetamide (8.6 mg, 21.80 μmol, 39.95% yield).

LCMS(ESI): [M+H]⁺ m/z: calcd 395.2; found 395.2; Rt=1.754 min.

### Example 206. N-(4-Amino-1H-pyrazolo[4,3-c]pyridin-7-yl)-2-oxo-2-[rac-(2R,5S)-2-[3-[2-(dimethylamino)ethyl]phenyl]-5-methyl-1-piperidyl]acetamide (Compound 230)

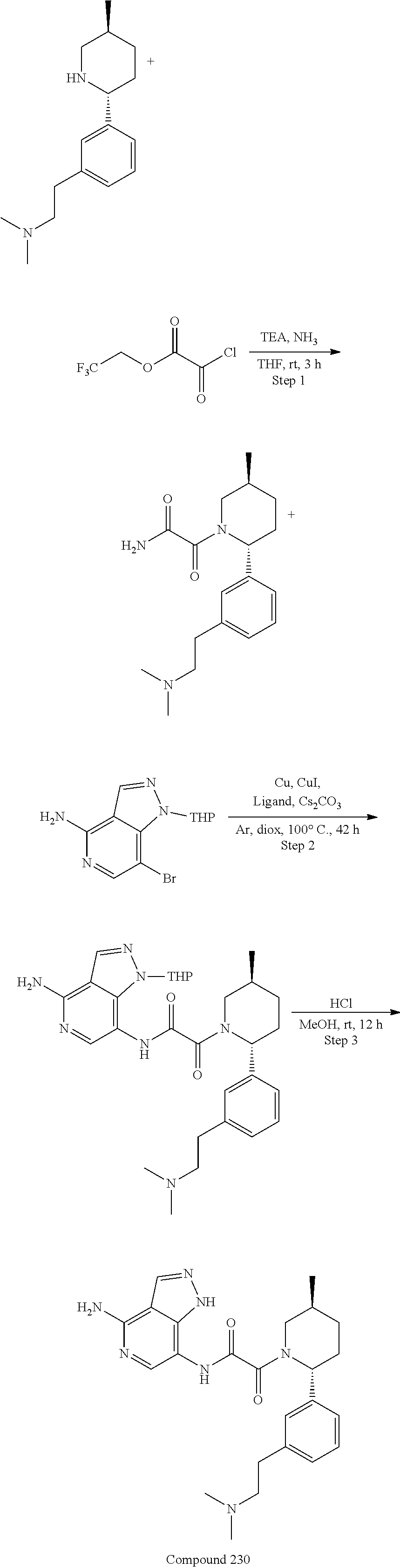

### Step 1: The Synthesis of 2-Oxo-2-[rac-(2R,5S)-2-[3-[2-(dimethylamino)ethyl]phenyl]-5-methyl-1-piperidyl]acetamide

To a solution of N,N-dimethyl-2-[3-[rac-(2R,5S)-5-methyl-2-piperidyl]phenyl]ethanamine (1.05 g, 4.26 mmol) and Triethylamine (862.45 mg, 8.52 mmol, 1.19 mL) in THF (20 mL), 2,2,2-trifluoroethyl 2-chloro-2-oxo-acetate (1.22 g, 6.39 mmol) was added portionwise at 25° C. The resulting mixture was stirred for 2 hr. LCMS showed full conversion of SM. Through resulting mixture Ammonia (72.58 mg, 4.26 mmol) was bubbled during 20 min at 25° C. The formed precipitate was filtered off, washed with THF (30 ml) and the solvent was evaporated in vacuo to give 1.8 g of crude product. 0.9 g of crude was submitted for reverse phase HPLC (SYSTEM 30-80% 0-5 min H₂O/MeOH/0.1% NH₄OH, flow: 30 mL/min (loading pump 4 mL/min methanol); column: XBridge C18 100×19 mm, 5 um) to give 2-oxo-2-[rac-(2R,5S)-2-[3-[2-(dimethylamino)ethyl]phenyl]-5-methyl-1-piperidyl]acetamide (0.4 g, 1.26 mmol, 29.57% yield) in 2 fractions:
1st: 173 mg (90% LCMS)
2nd: 236 mg (95% LCMS)

LCMS(ESI): [M+H]⁺ m/z: calcd 318.2; found 318.2; Rt=1.730 min.

### Step 2: The Synthesis of N-(4-Amino-2-tetrahydropyran-2-yl-pyrazolo[4,3-c]pyridin-7-yl)-2-oxo-2-[rac-(2R,5S)-2-[3-[2-(dimethylamino)ethyl]phenyl]-5-methyl-1-piperidyl]acetamide

A mixture of 2-oxo-2-[rac-(2R,5S)-2-[3-[2-(dimethylamino)ethyl]phenyl]-5-methyl-1-piperidyl]acetamide (236 mg, 743.48 μmol), 7-bromo-2-tetrahydropyran-2-yl-pyrazolo[4,3-c]pyridin-4-amine (330 mg, 1.11 mmol), copper (5 mg, 78.68 μmol), Copper (I) iodide (120 mg, 630.09 μmol, 21.35 μL), caesium carbonate (363.36 mg, 1.12 mmol) and rac-(1R,2R)—N1,N2-dimethylcyclohexane-1,2-diamine (120 mg, 843.64 μmol) in 1,4-dioxane (6.00 mL) was stirred in a sealed vial under argon at 105° C. for 42 hr. The resulting mixture was cooled down, diluted with DMSO (1 mL) and submitted to reverse phase HPLC (column: XBridge OBD C18 100×30 mm; mobile phase: 50-100% 0-1-5 min H₂O/MeOH/NH₄OH; flow rate: 30 mL/min (loading pump 4 mL/min acetonitrile) to afford N-(4-amino-2-tetrahydropyran-2-yl-pyrazolo[4,3-c]pyridin-7-yl)-2-oxo-2-[rac-(2R,5S)-2-[3-[2-(dimethylamino)ethyl]phenyl]-5-methyl-1-piperidyl]acetamide (70 mg, 131.17 μmol, 17.64% yield) as light-brown gum.

LCMS(ESI): [M+H]⁺ m/z: calcd 534.2; found 534.2; Rt=2.270 min.

### Step 3: The Synthesis of N-(4-Amino-1H-pyrazolo[4,3-c]pyridin-7-yl)-2-oxo-2-[rac-(2R,5S)-2-[3-[2-(dimethylamino)ethyl]phenyl]-5-methyl-1-piperidyl]acetamide (Compound 230)

Hydrogen chloride solution 4.0M in dioxane (1.05 g, 4.00 mmol, 1 mL, 13.9% purity) was added to a stirred solution of N-(4-amino-2-tetrahydropyran-2-yl-pyrazolo[4,3-c]pyridin-7-yl)-2-oxo-2-[rac-(2R,5S)-2-[3-[2-(dimethylamino)ethyl]phenyl]-5-methyl-1-piperidyl]acetamide (70 mg, 131.17 μmol) in methanol (1 mL) at 25° C. The resulting solution was stirred at 25° C. for 12 hr, then concentrated to dryness in vacuo and the residue was submitted to reverse phase HPLC (column: Chromatorex 18 SMB100-5T 100×19 mm 5 um; mobile phase: 5-5-45% 0-1-5 min H₂O/ACN/0.1% FA; flow: 30 mL/min (loading pump 4 mL/min H₂O)) to afford Compound 230 N-(4-amino-1H-pyrazolo[4,3-c]pyridin-7-yl)-2-oxo-2-[rac-(2R,5S)-2-[3-[2-(dimethylamino)ethyl]phenyl]-5-methyl-1-piperidyl]acetamide (14.9 mg, 30.07 μmol, 22.92% yield, HCOOH) as light-yellow solid.

¹H NMR (600 MHz, dmso) δ 0.16-1.08 (m, 3H), 1.18-1.44 (m, 1H), 1.46-2.08 (m, 3H), 2.09-2.30 (m, 7H), 2.51-2.58 (m, 2H), 2.67-2.76 (m, 2H), 3.21-3.26 (m, 1H), 3.70-4.10 (m, 1H), 5.25-5.71 (m, 1H), 6.56-7.69 (m, 6H), 7.71-8.39 (m, 3H), 9.49-10.66 (m, 1H), 12.26-13.61 (m, 1H).

LCMS(ESI): [M+H]⁺ m/z: calcd 450.2; found 450.2; Rt=1.562 min.

### Example 207. N-(4-Amino-1H-pyrazolo[4,3-c]pyridin-7-yl)-2-oxo-2-[rac-(2R,5S)-2-(2-isopropylindazol-5-yl)-5-methyl-1-piperidyl]acetamide (Compound 231) rac

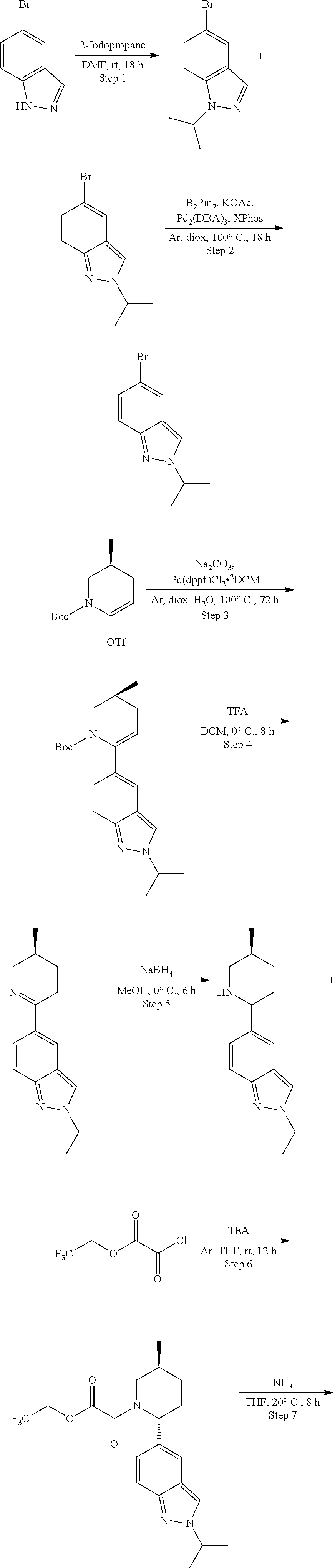

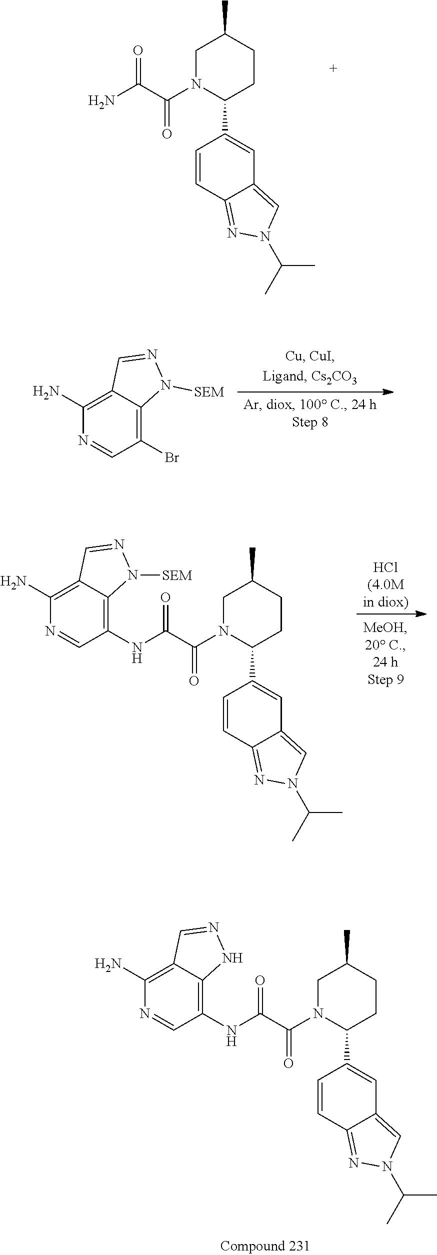 [SMILES: CC(C)n1cc2cc([C@H]3CC[C@H](C)CN3C(=O)C(N)=O)ccc2n1 ; C[Si](C)(C)CCOCn1ncc2c(N)ncc(Br)c21 ; CC(C)n1cc2cc([C@H]3CC[C@H](C)CN3C(=O)C(=O)Nc3cnc(N)c4cnn(COCC[Si](C)(C)C)c34)ccc2n1 ; "HCl(4.0Min diox)" ; CC(C)n1cc2cc([C@H]3CC[C@H](C)CN3C(=O)C(=O)Nc3cnc(N)c4cn[nH]c34)ccc2n1]

### Step 1: The Synthesis of 5-Bromo-1-isopropyl-indazole and 5-Bromo-2-isopropyl-indazole

To a solution of 5-bromo-1H-indazole (20 g, 101.51 mmol) in DMF (150 mL) was 5 added Potassium carbonate, anhydrous, 99% (42.09 g, 304.52 mmol, 18.38 mL). The resulting suspension was stirred for 1 h at 25° C., then 2-iodopropane (22.43 g, 131.96 mmol, 13.19 mL) was added dropwise and resulting reaction mixture was stirred for 16 h at 25° C. After 16 hr, the reaction mixture was quenched with ethylacetate (200 ml) and extracted with brine (3*150 mL). The organic layer was dried over Na₂SO₄ and evaporated under reduced pressure. The crude product was purified by column chromatography (Interchim; 330 g SiO2, petroleum ether/ethyl acetate with ethyl acetate from 5˜20%, flow rate=130 mL/min, Rv=4.5-6 CV) affording 5-bromo-1-isopropyl-indazole (8.0 g, 32.96% yield) and 5-bromo-2-isopropyl-indazole (5.0 g, 20.60% yield).

### 5-Bromo-1-isopropyl-indazole

¹H NMR (500 MHz, CDCl₃) δ (ppm) 1.56 (d, 6H), 4.80 (sept, 1H), 7.31 (d, 1H), 7.42 (d, 1H), 7.86 (s, 1H), 7.93 (s, 1H).

### 5-Bromo-2-isopropyl-indazole

¹H NMR (600 MHz, CDCl₃) δ (ppm) 1.64 (d, 1H), 4.77 (sept, 1H), 7.31 (d, 1H), 7.60 (d, 1H), 7.80 (s, 1H), 7.89 (s, 1H).

### Step 2: The Synthesis of 2-isopropyl-5-(4,4,5,5-tetramethyl-1,3,2-dioxaborolan-2-yl)indazole

Potassium Acetate (4.27 g, 43.49 mmol, 2.72 mL) was added to a solution of 5-bromo-2-isopropyl-indazole (5.2 g, 21.75 mmol) and 4,4,5,5-tetramethyl-2-(4,4,5,5-tetramethyl-1,3,2-dioxaborolan-2-yl)-1,3,2-dioxaborolane (7.18 g, 28.27 mmol) in Dioxane (198.64 mL). Reaction flask was evacuated and refilled with argon 3 times. Then Pd2(dba)3 (199.14 mg, 217.47 μmol) and XPhos (207.34 mg, 434.94 μmol) was added under stream of argon. Resulting mixture was stirred at 100° C. for 18 hr under inert atmosphere, then cooled and evaporated in vacuo poured into water (120 ml) and extracted with DCM (2×50 ml) and dried in vacuo to leave 9 g of crude product, 9 g of which was purification by column chromatography on silica gel using Hexane/MTBE gradient (10-100% MTBE) to afford product 2-isopropyl-5-(4,4,5,5-tetramethyl-1,3,2-dioxaborolan-2-yl)indazole (6 g, 20.97 mmol, 96.41% yield).

### Step 3: The Synthesis of tert-Butyl rac-(3S)-6-(2-isopropylindazol-5-yl)-3-methyl-3,4-dihydro-2H-pyridine-1-carboxylate

Sodium carbonate (1.11 g, 10.48 mmol, 438.82 μL) was added to the solution of 2-isopropyl-5-(4,4,5,5-tetramethyl-1,3,2-dioxaborolan-2-yl)indazole (3 g, 10.48 mmol) and tert-butyl rac-(3S)-3-methyl-6-(trifluoromethylsulfonyloxy)-3,4-dihydro-2H-pyridine-1-carboxylate (3.62 g, 10.48 mmol) in Dioxane (60 mL) and Water (2 mL). Reaction flask was evacuated and refilled with argon 3 times. Then, PdCl2*dppf*dcm (10.48 mmol) was added under stream of argon. Resulting mixture was stirred at 100° C. for 72 hr under inert atmosphere. Then, it was concentrated under reduced pressure and residue was extracted with boiling MTBE (60 ml). The combined organic extracts, dried over sodium sulphate and evaporated in vacuo to afford tert-butyl rac-(3S)-6-(2-isopropylindazol-5-yl)-3-methyl-3,4-dihydro-2H-pyridine-1-carboxylate (4 g, crude).

LCMS(ESI): [M+H]⁺ m/z: calcd 356.2; found 356.2; Rt=4.30 min.

### Step 4: The Synthesis of 2-Isopropyl-5-[rac-(3S)-3-methyl-2,3,4,5-tetrahydropyridin-6-yl]indazole

Trifluoroacetic acid (20 g, 175.41 mmol, 13.51 mL) was added to the solution of tert-butyl rac-(3S)-6-(2-isopropylindazol-5-yl)-3-methyl-3,4-dihydro-2H-pyridine-1-carboxylate (4 g, 11.25 mmol) in Dichloromethane (20 mL). Resulting mixture was stirred at 0° C. for 8 hr. Then, volatiles were removed under reduced pressure and residue was partitioned between 20% aq. K₂CO₃ solution (30 mL) and ethyl acetate (50 mL). Organic layer was separated, dried over Na₂SO₄ and concentrated in vacuo, affording 2-isopropyl-5-[rac-(3S)-3-methyl-2,3,4,5-tetrahydropyridin-6-yl]indazole (0.8 g, 3.13 mmol, 27.84% yield).

LCMS(ESI): [M+H]⁺ m/z: calcd 256.2; found 256.2; Rt=0.636 min.

### Step 5: The Synthesis of 2-Isopropyl-5-[rac-(2R,5S)-5-methyl-2-piperidyl]indazole

Sodium borohydride (237.03 mg, 6.27 mmol, 220.70 μL) was added in one portion to a stirred solution of 2-isopropyl-5-[rac-(3S)-3-methyl-2,3,4,5-tetrahydropyridin-6-yl]indazole (0.8 g, 3.13 mmol) in Methanol (10 mL) at 0° C. The resulting mixture was stirred at 0° C. for 6 hr, and then evaporated in vacuo. The residue was diluted with water (20 mL) and extracted with dichloromethane (2*20 mL). The combined organic extracts were dried over sodium sulphate and evaporated in vacuo to afford 2-isopropyl-5-[rac-(2R,5S)-5-methyl-2-piperidyl]indazole (0.7 g, 2.72 mmol, 86.81% yield).

LCMS(ESI): [M+H]⁺ m/z: calcd 258.2; found 258.4; Rt=1.958 min.

### Step 6: The Synthesis of 2,2,2-Trifluoroethyl 2-oxo-2-[rac-(2R,5S)-2-(2-isopropylindazol-5-yl)-5-methyl-1-piperidyl]acetate

To a solution of 2-isopropyl-5-[rac-(2R,5S)-5-methyl-2-piperidyl]indazole (0.7 g, 2.72 mmol) and TEA (412.82 mg, 4.08 mmol, 568.63 μL) in THF (20 mL) was added 2,2,2-trifluoroethyl 2-chloro-2-oxo-acetate (621.76 mg, 3.26 mmol) dropwise at 0° C. under argon. The reaction mixture was then stirred for 12 hr at r.t., then evaporated in vacuo to give 2,2,2-trifluoroethyl 2-oxo-2-[rac-(2R,5S)-2-(2-isopropylindazol-5-yl)-5-methyl-1-piperidyl]acetate (0.68 g, 1.65 mmol, 60.77% yield).

LCMS(ESI): [M+H]⁺ m/z: calcd 412.0; found 412.0; Rt=3.40 min.

### Step 7: The Synthesis of 2-Oxo-2-[rac-(2R,5S)-2-(2-isopropylindazol-5-yl)-5-methyl-1-piperidyl]acetamide

2,2,2-Trifluoroethyl 2-oxo-2-[rac-(2R,5S)-2-(2-isopropylindazol-5-yl)-5-methyl-1-piperidyl]acetate (0.68 g, 1.65 mmol) was dissolved in THF (15 mL) and was blow ammonium (29.82 mg, 1.65 mmol). Resulting solution was stirred at 20° C. for 8 hr. The resulting mixture was evaporated in vacuo and residue was triturated with THF (20 ml*2), filtered and combined organic was evaporated in vacuo and was purified by RP-HPLC (column: Chromatex C18 5 um 130 A; 40-45% 0-5 min H₂O/CH₃CN flow: 30 mL/min) to give 2-oxo-2-[rac-(2R,5S)-2-(2-isopropylindazol-5-yl)-5-methyl-1-piperidyl]acetamide (0.0261 g, 79.47 μmol, 4.81% yield).

LCMS(ESI): [M+H]⁺ m/z: calcd 329.2; found 329.2; Rt=2.858 min.

### Step 8: The Synthesis of N-[4-Amino-1-(2-trimethylsilylethoxymethyl)pyrazolo[4,3-c]pyridin-7-yl]-2-oxo-2-[rac-(2R,5S)-2-(2-isopropylindazol-5-yl)-5-methyl-1-piperidyl]acetamide

Copper (2.23 mg, 35.02 μmol), Copper (I) iodide (66.69 mg, 350.17 μmol, 11.87 μL), caesium carbonate (456.38 mg, 1.40 mmol) was added to a stirred solution of 2-oxo-2-[rac-(2R,5S)-2-(2-isopropylindazol-5-yl)-5-methyl-1-piperidyl]acetamide (0.23 g, 700.35 μmol), 7-bromo-1-(2-trimethylsilylethoxymethyl)pyrazolo[4,3-c]pyridin-4-amine (252.45 mg, 735.37 μmol), rac-(1R,2R)—N1,N2-dimethylcyclohexane-1,2-diamine (49.81 mg, 350.17 μmol) in 1,4-dioxane (7.00 mL) under Ar atmosphere and stirred at 110° C. for 48 hr in closed vial. Reaction mixture was filtered, solid washed with dioxane (2×3 mL), filtrate concentrated and the crude product submitted to HPLC (column: XBridge C18 100×19 mm, 5 um; 50-75% 0-5 min H₂O/ACN/0.1% NH₄OH, flow rate: 30 mL/min) to give N-[4-amino-1-(2-trimethylsilylethoxymethyl)pyrazolo[4,3-c]pyridin-7-yl]-2-oxo-2-[rac-(2R,5S)-2-(2-isopropylindazol-5-yl)-5-methyl-1-piperidyl]acetamide (0.11 g, 186.19 μmol, 26.59% yield).

LCMS(ESI): [M+H]⁺ m/z: calcd 592.2; found 592.4; Rt=2.539 min.

### Step 8: The Synthesis of N-(4-Amino-1H-pyrazolo[4,3-c]pyridin-7-yl)-2-oxo-2-[rac-(2R,5S)-2-(2-isopropylindazol-5-yl)-5-methyl-1-piperidyl]acetamide (Compound 231)

Hydrogen chloride solution 4.0M in dioxane (678.85 mg, 18.62 mmol, 848.57 μL) was added to a solution of N-[4-amino-1-(2-trimethylsilylethoxymethyl)pyrazolo[4,3-c]pyridin-7-yl]-2-oxo-2-[rac-(2R,5S)-2-(2-isopropylindazol-5-yl)-5-methyl-1-piperidyl]acetamide (0.11 g, 186.19 μmol) in Methanol (7.09 mL) and stirred at 20° C. for 24 hr. Volatiles was evaporated in vacuo, the residue triturred with IPA (5 mL), filtered, washed with IPA (5 mL), and submitted to HPLC (column: Chromatorex 18 SMB100-5T 100×19 mm 5 um; 5-5-30% 0-1-6 min H2O/ACN/0.1% FA, flow rate: 30 mL/min) to give N-(4-amino-1H-pyrazolo[4,3-c]pyridin-7-yl)-2-oxo-2-[rac-(2R,5S)-2-(2-isopropylindazol-5-yl)-5-methyl-1-piperidyl]acetamide (42 mg, 82.91 μmol, 44.53% yield, HCOOH).

¹H NMR (600 MHz, dmso) δ 0.98-1.06 (m, 3H), 1.22-1.41 (m, 1H), 1.51-1.55 (m, 6H), 1.74-1.93 (m, 2H), 2.02-2.15 (m, 1H), 2.22-2.33 (m, 1H), 2.67-2.97 (m, 1H), 3.75-4.11 (m, 1H), 4.74-4.88 (m, 1H), 5.32-5.77 (m, 1H), 6.71-7.00 (m, 2H), 7.15-7.29 (m, 1H), 7.55-7.64 (m, 2H), 7.65-7.80 (m, 1H), 8.19-8.27 (m, 1H), 8.30-8.40 (m, 1H), 9.50-10.67 (m, 1H), 12.61-13.38 (m, 1H).

LCMS(ESI): [M+H]⁺ m/z: calcd 461.2; found 461.4; Rt=2.652 min.

### Example 208. The Synthesis of N-(4-Amino-1H-pyrazolo[4,3-c]pyridin-7-yl)-2-oxo-2-[rac-(2R,5S)-2-(1-isopropylindazol-5-yl)-5-methyl-1-piperidyl]acetamide (Compound 266)

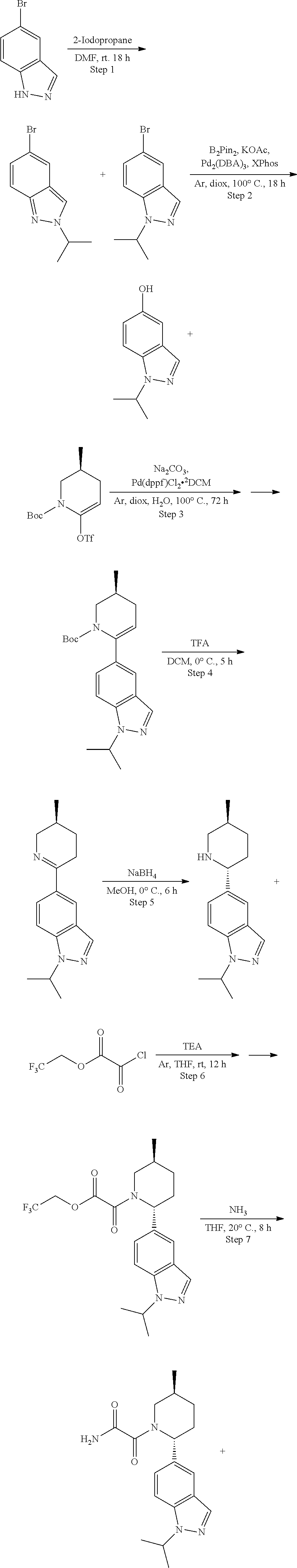

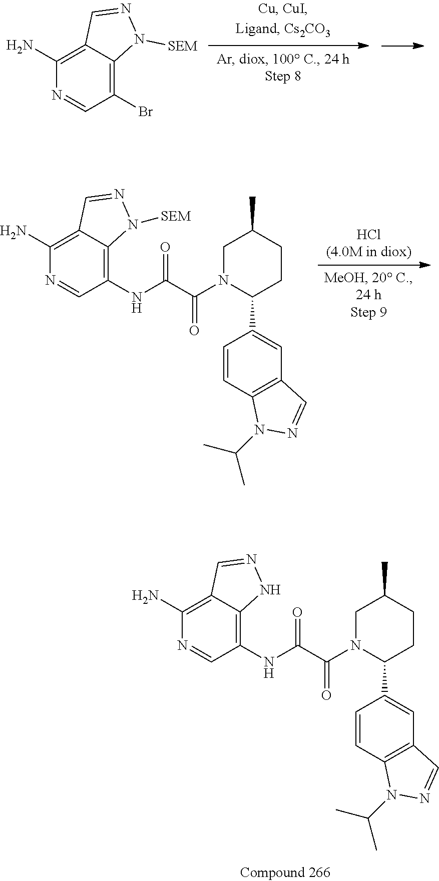 [SMILES: C[Si](C)(C)CCOCn1ncc2c(N)ncc(Br)c21 ; CC(C)n1ncc2cc([C@H]3CC[C@H](C)CN3C(=O)C(=O)Nc3cnc(N)c4cnn(COCC[Si](C)(C)C)c34)ccc21 ; "HCl(4.0M in diox)" ; CC(C)n1ncc2cc([C@H]3CC[C@H](C)CN3C(=O)C(=O)Nc3cnc(N)c4cn[nH]c34)ccc21]

### Step 1 is the Same as for Compound 231

### Step 2: The Synthesis of 1-Isopropyl-5-(4,4,5,5-tetramethyl-1,3,2-dioxaborolan-2-yl)indazole

Potassium Acetate (5.75 g, 58.55 mmol, 3.66 mL) was added to a solution of 5-bromo-1-isopropyl-indazole (7 g, 29.28 mmol) and 4,4,5,5-tetramethyl-2-(4,4,5,5-tetramethyl-1,3,2-dioxaborolan-2-yl)-1,3,2-dioxaborolane (9.66 g, 38.06 mmol) in Dioxane (148.17 mL). Reaction flask was evacuated and refilled with argon 3 times. Then Pd2(dba)3 (268.08 mg, 292.75 μmol) and XPhos (279.12 mg, 585.50 μmol) was added under stream of argon. Resulting mixture was stirred at 100° C. for 18 hr under inert atmosphere, then cooled and evaporated in vacuo poured into water (120 ml) and extracted with DCM (2×50 mL) and dried in vacuo to leave 9 g of crude product, 9 g of which was purification by column chromatography on silica gel using Hexane/MTBE gradient (10-100% MTBE) to afford product 1-isopropyl-5-(4,4,5,5-tetramethyl-1,3,2-dioxaborolan-2-yl)indazole (6 g, 20.97 mmol, 71.62% yield).

¹H NMR (500 MHz, CDCl₃) δ (ppm) 1.35 (m, 12H), 1.59 (m, 6H), 4.85 (sept, 1H), 7.42 (m, 1H), 7.76 (m, 1H), 8.01 (m, 1H), 8.26 (m, 1H).

### Step 3: The Synthesis of tert-Butyl rac-(3S)-6-(1-isopropylindazol-5-yl)-3-methyl-3,4-dihydro-2H-pyridine-1-carboxylate

Sodium carbonate (740.73 mg, 6.99 mmol, 292.55 μL) was added to the solution of 1-isopropyl-5-(4,4,5,5-tetramethyl-1,3,2-dioxaborolan-2-yl)indazole (2 g, 6.99 mmol) and tert-butyl rac-(3S)-3-methyl-6-(trifluoromethylsulfonyloxy)-3,4-dihydro-2H-pyridine-1-carboxylate (4.83 g, 13.98 mmol) in Dioxane (40 mL) and Water (4 mL). Reaction flask was evacuated and refilled with argon 3 times. Then, PdCl₂*dppf*dcm (570.72 mg, 698.87 μmol) was added under stream of argon. Resulting mixture was stirred at 100° C. for 72 hr under inert atmosphere. Then, it was concentrated under reduced pressure and residue was extracted with boiling MTBE (60 ml). The combined organic extracts dried over sodium sulphate and evaporated in vacuo to afford tert-butyl rac-(3S)-6-(1-isopropylindazol-5-yl)-3-methyl-3,4-dihydro-2H-pyridine-1-carboxylate (3 g, crude).

LCMS(ESI): [M+H]⁺ m/z: calcd 356.2; found 356.4; Rt=4.294 min.

### Step 4: The Synthesis of 1-Isopropyl-5-[rac-(3S)-3-methyl-2,3,4,5-tetrahydropyridin-6-yl]indazole

Trifluoroacetic acid (20 g, 175.41 mmol, 13.51 mL) was added to the solution of tert-butyl rac-(3S)-6-(1-isopropylindazol-5-yl)-3-methyl-3,4-dihydro-2H-pyridine-1-carboxylate (3 g, 8.44 mmol) in Dichloromethane (20 mL). Resulting mixture was stirred at 0° C. for 5 hr. Then, volatiles were removed under reduced pressure and residue was partitioned between 20% aq. K₂CO₃ solution (30 ml) and ethyl acetate (50 ml). Organic layer was separated, dried over Na₂SO₄ and concentrated in vacuo, affording 1-isopropyl-5-[rac-(3S)-3-methyl-2,3,4,5-tetrahydropyridin-6-yl]indazole (1.3 g, 5.09 mmol, 60.32% yield).

LCMS(ESI): [M+H]⁺ m/z: calcd 256.4; found 256.4; Rt=2.259 min.

### Step 5: The Synthesis of 1-Isopropyl-5-[rac-(2R,5S)-5-methyl-2-piperidyl]indazole

Sodium borohydride (385.18 mg, 10.18 mmol, 358.64 μL) was added in one portion to a stirred solution of 1-isopropyl-5-[rac-(3S)-3-methyl-2,3,4,5-tetrahydropyridin-6-yl]indazole (1.3 g, 5.09 mmol) in Methanol (9.82 mL) at 0° C. The resulting mixture was stirred at 0° C. for 6 hr, and then evaporated in vacuo. The residue was diluted with water (20 mL) and extracted with dichloromethane (2*20 mL). The combined organic extracts were dried over sodium sulphate and evaporated in vacuo to afford 1-isopropyl-5-[rac-(2R,5S)-5-methyl-2-piperidyl]indazole (0.6 g, 2.33 mmol, 45.79% yield).

LCMS(ESI): [M+H]⁺ m/z: calcd 258.4; found 258.4; Rt=1.968 min.

### Step 6: The Synthesis of 2,2,2-Trifluoroethyl 2-oxo-2-[rac-(2R,5S)-2-(1-isopropylindazol-5-yl)-5-methyl-1-piperidyl]acetate

To a solution of 1-isopropyl-5-[rac-(2R,5S)-5-methyl-2-piperidyl]indazole (0.6 g, 2.33 mmol) and TEA (353.85 mg, 3.50 mmol, 487.39 μL) in THF (20.08 mL) was added 2,2,2-trifluoroethyl 2-chloro-2-oxo-acetate (532.94 mg, 2.80 mmol) dropwise at 0° C. under argon. The reaction mixture was then stirred for 12 hr at r.t., then evaporated in vacuo to give 2,2,2-trifluoroethyl 2-oxo-2-[rac-(2R,5S)-2-(1-isopropylindazol-5-yl)-5-methyl-1-piperidyl]acetate (0.68 g, 1.65 mmol, 70.90% yield).

LCMS(ESI): [M+H]⁺ m/z: calcd 412.2; found 412.2; Rt=3.963 min.

### Step 7: The Synthesis of 2-Oxo-2-[rac-(2R,5S)-2-(1-isopropylindazol-5-yl)-5-methyl-1-piperidyl]acetamide

2,2,2-Trifluoroethyl 2-oxo-2-[rac-(2R,5S)-2-(1-isopropylindazol-5-yl)-5-methyl-1-piperidyl]acetate (0.68 g, 1.65 mmol) was dissolved in THF (20 mL) and was blow ammonium (596.34 mg, 35.02 mmol). Resulting solution was stirred at 20° C. for 8 hr. The resulting mixture was evaporated in vacuo and residue was triturated with THF (20 ml*2), filtered and combined organic was evaporated in vacuo and was purified by RP-HPLC (column: Chromatex C18 5 um 130 A; 35-35-90% 0-5 min H₂O/CH₃OH flow: 30 mL/min) to give 2-oxo-2-[rac-(2R,5S)-2-(1-isopropylindazol-5-yl)-5-methyl-1-piperidyl]acetamide (0.160 g, crude).

LCMS(ESI): [M+H]⁺ m/z: calcd 329.2; found 329.2; Rt=3.021 min.

### Step 8: The Synthesis of N-[4-Amino-1-(2-trimethylsilylethoxymethyl)pyrazolo[4,3-c]pyridin-7-yl]-2-oxo-2-[rac-(2R,5S)-2-(1-isopropylindazol-5-yl)-5-methyl-1-piperidyl]acetamide

Copper (30.96 mg, 487.20 μmol), Copper (I) iodide (92.79 mg, 487.20 μmol, 16.51 μL), caesium carbonate (158.74 mg, 487.20 μmol) was added to a stirred solution of 2-oxo-2-[rac-(2R,5S)-2-(1-isopropylindazol-5-yl)-5-methyl-1-piperidyl]acetamide (0.16 g, 487.20 μmol), 7-bromo-1-(2-trimethylsilylethoxymethyl)pyrazolo[4,3-c]pyridin-4-amine (167.25 mg, 487.20 μmol), rac-(1R,2R)—N1,N2-dimethylcyclohexane-1,2-diamine (69.30 mg, 487.20 μmol) in 1,4-dioxane (7 mL) under Ar atmosphere and stirred at 110° C. for 48 hr in closed vial. Reaction mixture was filtered, solid washed with dioxane (2×3 mL), filtrate concentrated to give crude N-[4-amino-1-(2-trimethylsilylethoxymethyl)pyrazolo[4,3-c]pyridin-7-yl]-2-oxo-2-[rac-(2R,5S)-2-(1-isopropylindazol-5-yl)-5-methyl-1-piperidyl]acetamide (0.28 g, 473.94 μmol, 97.28% yield).

LCMS(ESI): [M+H]⁺ m/z: calcd 591.2; found 591.4; Rt=1.065 min.

### Step 9: The Synthesis of N-(4-Amino-1H-pyrazolo[4,3-c]pyridin-7-yl)-2-oxo-2-[rac-(2R,5S)-2-(1-isopropylindazol-5-yl)-5-methyl-1-piperidyl]acetamide (Compound 266)

Hydrogen chloride solution 4.0M in dioxane (1.73 g, 47.39 mmol, 2.16 mL) was added to a solution of N-[4-amino-1-(2-trimethylsilylethoxymethyl)pyrazolo[4,3-c]pyridin-7-yl]-2-oxo-2-[rac-(2R,5S)-2-(1-isopropylindazol-5-yl)-5-methyl-1-piperidyl]acetamide (0.28 g, 473.94 μmol) in Methanol (5.78 mL) and stirred at 20° C. for 24 hr. Volatiles was evaporated in vacuo, the residue was mixed with IPA (5 mL), filtered, washed with IPA (5 mL), and submitted to HPLC (column: Chromatorex 18 SMB100-5T 100×19 mm 5 um; 10-25% 0-5 min H2O/ACN/0.1% FA, flow rate: 30 ml/min) to give N-(4-amino-1H-pyrazolo[4,3-c]pyridin-7-yl)-2-oxo-2-[rac-(2R,5S)-2-(1-isopropylindazol-5-yl)-5-methyl-1-piperidyl]acetamide (26 mg, 51.33 μmol, 10.83% yield, HCOOH).

¹H NMR (600 MHz, dmso) δ 0.94-1.09 (m, 3H), 1.26-1.42 (m, 1H), 1.42-1.49 (m, 6H), 1.55-2.33 (m, 4H), 2.65-3.08 (m, 1H), 3.71-4.09 (m, 1H), 4.81-5.03 (m, 1H), 5.03-5.75 (m, 1H), 6.24-7.02 (m, 2H), 7.07-7.66 (m, 2H), 7.65-7.76 (m, 2H), 7.90-8.17 (m, 2H), 8.16-8.29 (m, 1H), 9.57-10.63 (m, 1H), 12.06-13.77 (m, 2H).

LCMS(ESI): [M+H]⁺ m/z: calcd 461.4; found 461.4; Rt=2.370 min.

### Example 209. The Synthesis of N-(4-Amino-1H-pyrazolo[4,3-c]pyridin-7-yl)-2-oxo-2-[rac-(2R,5S)-2-(2,5-dimethylpyrazol-3-yl)-5-methyl-1-piperidyl]acetamide (Compound 259)

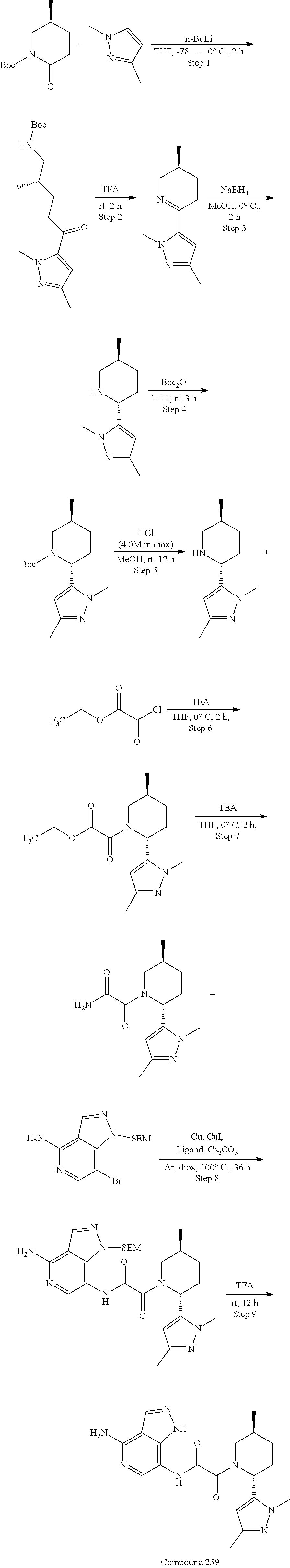

### Step 1: The Synthesis of tert-Butyl N-[rac-(2S)-5-(2,5-dimethylpyrazol-3-yl)-2-methyl-5-oxo-pentyl]carbamate

n-Butyllithium (5.75 g, 20.63 mmol, 8.45 mL, 23% purity) (2.5 M in hexane) was added dropwise at −78° C. to a stirred solution of 1,3-dimethylpyrazole (1.80 g, 18.76 mmol) in THF (50 mL). The resulting heavy suspension was stirred at −78° C. for 1 hr, then tert-butyl rac-(5S)-5-methyl-2-oxo-piperidine-1-carboxylate (4 g, 18.76 mmol) was added dropwise at −78° C. and the reaction mixture was allowed to warm to 0° C. and stirred for 1 hr. The resulting suspension was diluted with water (50 mL) and MTBE (100 mL), transferred to a separatory funnel, and the upper organic layer was separated. The aqueous layer was additionally extracted with MTBE (50 mL). The combined organic extracts were washed with water (20 ml), dried over sodium sulphate and evaporated in vacuo to afford tert-butyl N-[rac-(2S)-5-(2,5-dimethylpyrazol-3-yl)-2-methyl-5-oxo-pentyl]carbamate (5.7 g, 18.42 mmol, 98.23% yield) as yellow oil, which was used directly in the next step.

LCMS(ESI): [M+H]⁺ m/z: calcd 310.2; found 310.2; Rt=1.028 min.

### Step 2: The Synthesis of rac-(3S)-6-(2,5-Dimethylpyrazol-3-yl)-3-methyl-2,3,4,5-tetrahydropyridine

The solution of tert-butyl N-[rac-(2S)-5-(2,5-dimethylpyrazol-3-yl)-2-methyl-5-oxo-pentyl]carbamate (5.7 g, 18.42 mmol) in Trifluoroacetic acid (29.60 g, 259.60 mmol, 20 mL) was stirred at 25° C. for 2 hr, and then evaporated in vacuo. Crushed ice (20 g) was added to the residue and pH was adjusted to 10 with a 10% aqueous solution of sodium hydroxide. The resulting mixture was extracted with dichloromethane (2*30 mL). The combined organic extracts were dried over sodium sulphate and evaporated in vacuo to afford rac-(3S)-6-(2,5-dimethylpyrazol-3-yl)-3-methyl-2,3,4,5-tetrahydropyridine (2.4 g, 12.55 mmol, 68.11% yield) as yellow oil, which was used directly in the next step.

LCMS(ESI): [M+2H]⁺ m/z: calcd 193.2; found 193.2; Rt=0.413 min.

### Step 3: The Synthesis of rac-(2R,5S)-2-(2,5-Dimethylpyrazol-3-yl)-5-methyl-piperidine

Sodium Borohydride (474.71 mg, 12.55 mmol, 442.00 μL) was added in one portion to a stirred solution of rac-(3S)-6-(2,5-dimethylpyrazol-3-yl)-3-methyl-2,3,4,5-tetrahydropyridine (2.4 g, 12.55 mmol) in Methanol (40 mL) at 0° C. The resulting mixture was stirred at 0° C. for 2 hr, and then evaporated in vacuo. The residue was diluted with water (20 mL) and extracted with dichloromethane (2*40 mL). The combined organic extracts were dried over sodium sulphate and evaporated in vacuo to afford rac-(2R,5S)-2-(2,5-dimethylpyrazol-3-yl)-5-methyl-piperidine (2 g, 10.35 mmol, 82.46% yield).

LCMS(ESI): [M+H]⁺ m/z: calcd 194.2; found 194.2; Rt=0.553 min.

### Step 4: The Synthesis of tert-Butyl rac-(2R,5S)-5-methyl-2-[(3-methylpyrazol-1-yl)methyl]piperidine-1-carboxylate and tert-Butyl rac-(2R,5S)-2-(2,5-dimethylpyrazol-3-yl)-5-methyl-piperidine-1-carboxylate

A solution of rac-(2R,5S)-2-(2,5-dimethylpyrazol-3-yl)-5-methyl-piperidine (2 g, 10.35 mmol) and Di-tert-butyl dicarbonate (2.26 g, 10.35 mmol, 2.37 mL) in THF (29.99 mL) was stirred at 25° C. for 3 hr. The residue was purified by gradient chromatography (hexane-MTBE) to obtain ˜2 g mixture, which was re-purified by HPLC (Device (Mobile Phase, Column): SYSTEM 55-90% 0-5 min H2O/MeOH, flow: 30 mL/min (loading pump 4 mL/min methanol) target mass 293.41; column: Chromatorex 18 SMB100-5T 100×19 mm 5 um) to give tert-butyl rac-(2R,5S)-2-(2,5-dimethylpyrazol-3-yl)-5-methyl-piperidine-1-carboxylate (0.68 g, 2.32 mmol, 44.80% yield) and tert-butyl rac-(2R,5S)-5-methyl-2-[(3-methylpyrazol-1-yl)methyl]piperidine-1-carboxylate (0.94 g, 3.20 mmol, 61.93% yield). tert-Butyl rac-(2R,5S)-2-(2,5-dimethylpyrazol-3-yl)-5-methyl-piperidine-1-carboxylate:

LCMS(ESI): [M+H]⁺ m/z: calcd 294.2; found 294.2; Rt=1.314 min. tert-butyl rac-(2R,5S)-5-methyl-2-[(3-methylpyrazol-1-yl)methyl]piperidine-1-carboxylate:

LCMS(ESI): [M+H]⁺ m/z: calcd 294.2; found 294.2; Rt=1.397 min.

### Step 5: The Synthesis of rac-(2R,5S)-2-(2,5-Dimethylpyrazol-3-yl)-5-methyl-piperidine

A solution of tert-butyl rac-(2R,5S)-2-(2,5-dimethylpyrazol-3-yl)-5-methyl-piperidine-1-carboxylate (0.68 g, 2.32 mmol) in Methanol (10 mL) and Hydrogen chloride solution 4.0M in dioxane (5 g, 137.13 mmol, 6.25 mL) was stirred at 25° C. for 12 hr. The solvent was evaporated to give rac-(2R,5S)-2-(2,5-dimethylpyrazol-3-yl)-5-methyl-piperidine (0.6 g, 2.25 mmol, 97.25% yield, 2HCl).

LCMS(ESI): [M+H]⁺ m/z: calcd 194.2; found 194.2; Rt=0.658 min.

### Step 6 The Synthesis of 2,2,2-Trifluoroethyl 2-oxo-2-[rac-(2R,5S)-2-(2,5-dimethylpyrazol-3-yl)-5-methyl-1-piperidyl]acetate

To a solution of rac-(2R,5S)-2-(2,5-dimethylpyrazol-3-yl)-5-methyl-piperidine (0.6 g, 2.25 mmol, 2HCl) and Triethylamine (1.14 g, 11.27 mmol, 1.57 mL) in THF (30 mL), 2,2,2-trifluoroethyl 2-chloro-2-oxo-acetate (1.29 g, 6.76 mmol) was added portionwise at 0° C. The resulting mixture was stirred for 2 hr. LCMS showed full conversion of SM. The reaction mixture was directly used for the next step.

LCMS(ESI): [M+H]⁺ m/z: calcd 348.2; found 348.2; Rt=1.170 min.

### Step 7: The Synthesis of 2-Oxo-2-[rac-(2R,5S)-2-(2,5-dimethylpyrazol-3-yl)-5-methyl-1-piperidyl]acetamide

Through a solution of 2,2,2-trifluoroethyl 2-oxo-2-[rac-(2R,5S)-2-(2,5-dimethylpyrazol-3-yl)-5-methyl-1-piperidyl]acetate (0.75 g, 2.16 mmol) in THF (50 mL), ammonia was bubbled during 10 min at 0° C. The formed precipitate was filtered off, washed with THF (30 mL) and the solvent was evaporated in vacuo to give 2-oxo-2-[rac-(2R,5S)-2-(2,5-dimethylpyrazol-3-yl)-5-methyl-1-piperidyl]acetamide (0.5 g, 1.89 mmol, 87.60% yield).

LCMS(ESI): [M+H]⁺ m/z: calcd 265.2; found 265.2; Rt=0.800 min.

### Step 8: The Synthesis of N-[4-Amino-1-(2-trimethylsilylethoxymethyl)pyrazolo[4,3-c]pyridin-7-yl]-2-oxo-2-[rac-(2R,5S)-2-(2,5-dimethylpyrazol-3-yl)-5-methyl-1-piperidyl]acetamide

To a mixture of 2-oxo-2-[rac-(2R,5S)-2-(2,5-dimethylpyrazol-3-yl)-5-methyl-1-piperidyl]acetamide (0.3 g, 1.13 mmol), 7-bromo-1-(2-trimethylsilylethoxymethyl)pyrazolo[4,3-c]pyridin-4-amine (467.56 mg, 1.36 mmol), Copper (14.43 mg, 227.00 μmol), Copper (I) iodide (216.16 mg, 1.13 mmol, 38.46 μL), Cesium carbonate (739.60 mg, 2.27 mmol) and rac-(1R,2R)—N1,N2-dimethylcyclohexane-1,2-diamine (242.16 mg, 1.70 mmol), Dioxane (5 mL) was added. The resulting mixture was evacuated, refiled with Argon three time, heated at 100° C. for 36 hr and cooled. The inorganic precipitate was filtered and solution was purified by HPLC (Device (Mobile Phase, Column): SYSTEM 60-60-85% 0-1-6 min H₂O/MeOH/0.1% NH₄OH, flow: 30 mL/min (loading pump 4 mL/min MeOH); XBridge BEH C18 5 um 130 A) to give N-[4-amino-1-(2-trimethylsilylethoxymethyl)pyrazolo[4,3-c]pyridin-7-yl]-2-oxo-2-[rac-(2R,5S)-2-(2,5-dimethylpyrazol-3-yl)-5-methyl-1-piperidyl]acetamide (176 mg, 334.15 μmol, 29.44% yield).

LCMS(ESI): [M+H]⁺ m/z: calcd 527.2; found 527.2; Rt=1.197 min.

### Step 9: The Synthesis of N-(4-Amino-1H-pyrazolo[4,3-c]pyridin-7-yl)-2-oxo-2-[rac-(2R,5S)-2-(2,5-dimethylpyrazol-3-yl)-5-methyl-1-piperidyl]acetamide (Compound 259)

A solution of N-[4-amino-1-(2-trimethylsilylethoxymethyl)pyrazolo[4,3-c]pyridin-7-yl]-2-oxo-2-[rac-(2R,5S)-2-(2,5-dimethylpyrazol-3-yl)-5-methyl-1-piperidyl]acetamide (176 mg, 334.15 μmol) in Trifluoroacetic acid (3 g, 26.31 mmol, 2.03 mL) was stirred at 25° C. for 12 hr. The solvent was evaporated in vacuo and the residue was purified by HPLC (Device (Mobile Phase, Column): SYSTEM 15-65% 0-5 min H₂O/MeOH/0.1% NH₄OH, flow: 30 mL/min (loading pump 4 mL/min methanol); column: XBridge C18 100×19 mm, 5 um) to give N-(4-amino-1H-pyrazolo[4,3-c]pyridin-7-yl)-2-oxo-2-[rac-(2R,5S)-2-(2,5-dimethylpyrazol-3-yl)-5-methyl-1-piperidyl]acetamide (43 mg, 108.46 μmol, 32.46% yield).

¹H NMR (600 MHz, dmso) δ 0.84-1.05 (m, 3H), 1.24-1.44 (m, 1H), 1.56-1.96 (m, 2H), 1.96-2.03 (m, 1H), 2.04-2.14 (m, 4H), 2.96-3.23 (m, 1H), 3.54-3.65 (m, 1H), 3.66-3.71 (m, 2H), 3.72-4.09 (m, 1H), 5.52-5.75 (m, 1H), 6.09-6.29 (m, 1H), 6.52-6.99 (m, 2H), 7.32-7.75 (m, 1H), 8.06-8.25 (m, 1H), 9.50-10.50 (m, 1H), 12.46-13.40 (m, 1H).

LCMS(ESI): [M+H]⁺ m/z: calcd 397.2; found 397.2; Rt=2.127 min.

### Example 210. N-(4-Amino-1H-pyrazolo[4,3-c]pyridin-7-yl)-2-oxo-2-[rac-(2S)-2-[3-(diethylamino)phenyl]-1-piperidyl]acetamide (Compound 236) and N-(4-Amino-1H-pyrazolo[4,3-c]pyridin-7-yl)-2-oxo-2-[rac-(2R)-2-[3-(diethylamino)phenyl]-1-piperidyl]acetamide (Compound 276)

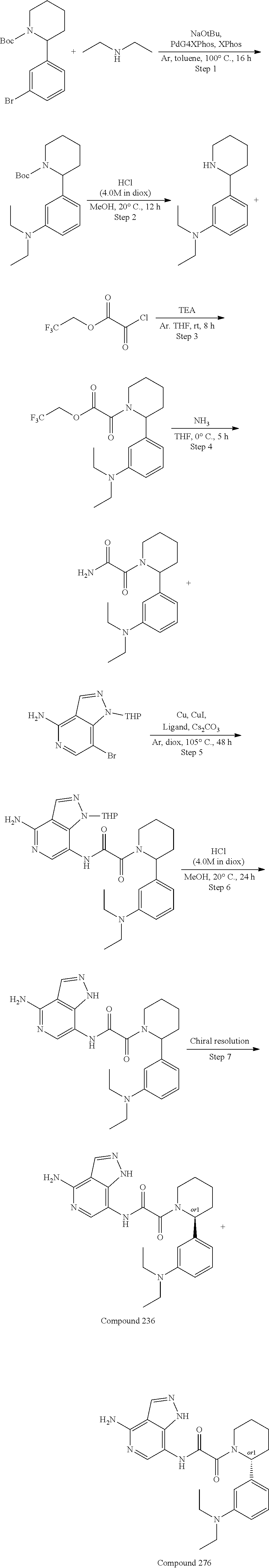

### Step 1: The Synthesis of tert-Butyl 2-[3-(diethylamino)phenyl]piperidine-1-carboxylate

tert-Butyl 2-(3-bromophenyl)piperidine-1-carboxylate (0.5 g, 1.47 mmol), N-ethylethanamine (322.42 mg, 4.41 mmol, 456.68 μL), XPhos (35.03 mg, 73.47 μmol) PdG4XPhos (63.22 mg, 73.47 μmol) and Sodium tert-butoxide (1.41 g, 2.94 mmol, 2 mL, 20% purity) were mixed in toluene (15 mL), purged with Ar for 15 minutes and then heated in the sealed tube at 100° C. for 16 hr. Final mixture was in vacuo and poured into water (20 ml) and extracted with DCM (2×20 ml). The combined organic extracts were washed with water (20 ml), dried over sodium sulphate and evaporated in vacuo to afford tert-butyl 2-[3-(diethylamino)phenyl]piperidine-1-carboxylate (0.35 g, 1.05 mmol, 71.64% yield).

### Step 2: The Synthesis of N,N-Diethyl-3-(2-piperidyl)aniline

Hydrogen chloride solution 4.0M in dioxane (12.45 g, 341.50 mmol, 15.56 mL) was added to a solution of tert-butyl 2-[3-(diethylamino)phenyl]piperidine-1-carboxylate (1.95 g, 5.87 mmol) in MeOH (24.44 mL). The reaction mixture was stirred at 20° C. for 12 hr, then evaporated and added to MTBE (15 mL) the resulting precipitate was filtered off, washed with MTBE (30 mL) and dried to afford N,N-diethyl-3-(2-piperidyl)aniline (1.25 g, 4.09 mmol, 69.81% yield, 2HCl).

¹H NMR (500 MHz, DMSO-d₆) δ (ppm) 1.05 (t, 6H), 1.88 (m, 9H), 3.49 (m, 2H), 3.50 (m, 2H), 7.47 (m, 4H), 9.28 (m, 1H), 9.61 (m, 1H).

### Step 3: The Synthesis of 2,2,2-Trifluoroethyl 2-[2-[3-(diethylamino)phenyl]-1-piperidyl]-2-oxo-acetate

To a solution of N,N-diethyl-3-(2-piperidyl)aniline (1.25 g, 4.11 mmol, 2HCl) and TEA (1.66 g, 16.43 mmol, 2.29 mL) in THF (29.61 mL) was added 2,2,2-trifluoroethyl 2-chloro-2-oxo-acetate (782.56 mg, 4.11 mmol) dropwise at 0° C. under argon. The reaction mixture was then stirred for 8 hr at r.t., then evaporated in vacuo to give 2,2,2-trifluoroethyl 2-[2-[3-(diethylamino)phenyl]-1-piperidyl]-2-oxo-acetate (1.59 g, 4.11 mmol, 100.00% yield).

LCMS(ESI): [M+H]⁺ m/z: calcd 387.2; found 387.2; Rt=2.972 min.

### Step 4: The Synthesis of 2-[2-[3-(Diethylamino)phenyl]-1-piperidyl]-2-oxo-acetamide

2,2,2-Trifluoroethyl 2-[2-[3-(diethylamino)phenyl]-1-piperidyl]-2-oxo-acetate (1.6 g, 4.14 mmol) was dissolved in THF (40 mL) and was blow ammonium (1.49 g, 82.81 mmol). Resulting solution was stirred at 0° C. for 5 hr. The resulting mixture was evaporated in vacuo and residue was triturated with THF (20 ml*2), filtered and combined organic was evaporated in vacuo to leave 1.3 g of crude product, 1.3 g of which was purification by column chromatography on silica gel using hexane/MTBE gradient (10-100% MTBE) to afford 2-[2-[3-(diethylamino)phenyl]-1-piperidyl]-2-oxo-acetamide (0.65 g, 2.14 mmol, 51.74% yield).

LCMS(ESI): [M+H]⁺ m/z: calcd 304.2; found 304.2; Rt=0.588 min.

### Step 5: The Synthesis of N-(4-Amino-1-tetrahydropyran-2-yl-pyrazolo[4,3-c]pyridin-7-yl)-2-[2-[3-(diethylamino)phenyl]-1-piperidyl]-2-oxo-acetamide

7-Bromo-1-tetrahydropyran-2-yl-pyrazolo[4,3-c]pyridin-4-amine (381.97 mg, 1.29 mmol), 2-[2-[3-(diethylamino)phenyl]-1-piperidyl]-2-oxo-acetamide (0.3 g, 988.80 μmol), Cu (3.14 mg, 49.44 μmol), CuI (175.65 mg, 922.31 μmol, 31.26 μL), cesium carbonate (483.26 mg, 1.48 mmol) and (1R,2R)—N1,N2-dimethylcyclohexane-1,2-diamine (175.65 mg, 1.23 mmol) were mixed in dioxane (6.00 mL), purged with Ar for 15 minutes and then heated in the sealed tube at 105° C. for 48 hr. Final mixture was filtered and dioxane was evaporated in vacuo. The crude product 0.6 g was purified by RP-HPLC (column: XBridge C18 5 um 130 A; 35-35-70% 0-1.3-5 min H₂O/CH₃CN/0.1% NH₄OH, flow: 30 mL/min) to give N-(4-amino-1-tetrahydropyran-2-yl-pyrazolo[4,3-c]pyridin-7-yl)-2-[2-[3-(diethylamino)phenyl]-1-piperidyl]-2-oxo-acetamide (0.0831 g, 159.92 μmol, 16.17% yield).

LCMS(ESI): [M+H]⁺ m/z: calcd 520.2; found 520.4; Rt=1.925 min.

### Step 6: The Synthesis of N-(4-Amino-1H-pyrazolo[4,3-c]pyridin-7-yl)-2-[2-[3-(diethylamino)phenyl]-1-piperidyl]-2-oxo-acetamide

Hydrogen chloride solution 4.0M in dioxane (2.40 g, 65.83 mmol, 3 mL) was added to a solution of N-(4-amino-1-tetrahydropyran-2-yl-pyrazolo[4,3-c]pyridin-7-yl)-2-[2-[3-(diethylamino)phenyl]-1-piperidyl]-2-oxo-acetamide (0.0831 g, 159.92 μmol) in MeOH (4 mL). The reaction mixture was stirred at 20° C. for 24 hr, then evaporated was purified by RP-HPLC (column: XBridge BEH18 SMB100-BT 100*19 mm; 50-85% 0-5 min H₂O/CH₃OH/0.1% NH₄OH, flow: 30 mL/min) to give N-(4-amino-1H-pyrazolo[4,3-c]pyridin-7-yl)-2-[2-[3-(diethylamino)phenyl]-1-piperidyl]-2-oxo-acetamide (0.0508 g, 116.64 μmol, 72.94% yield)

LCMS(ESI): [M+H]⁺ m/z: calcd 436.2; found 436.2; Rt=1.418 min.

### Step 7: The Synthesis of N-(4-Amino-1H-pyrazolo[4,3-c]pyridin-7-yl)-2-oxo-2-[rac-(2S)-2-[3-(diethylamino)phenyl]-1-piperidyl]acetamide (Compound 236) and N-(4-Amino-1H-pyrazolo[4,3-c]pyridin-7-yl)-2-oxo-2-[rac-(2R)-2-[3-(diethylamino)phenyl]-1-piperidyl]acetamide (Compound 276)

The N-(4-amino-1H-pyrazolo[4,3-c]pyridin-7-yl)-2-[2-[3-(diethylamino)phenyl]-1-piperidyl]-2-oxo-acetamide (0.0508 g, 116.64 μmol) was subjected to chiral HPLC purification (Column: Chiralpak IJ (250-20 mm-5 m); Mobile phase: Hexane-MeOH-IPA-DEA, 70-15-15-0.1 Flow Rate: 12 mL/min) to give the two individual enantiomers Compound 236-N-(4-amino-1H-pyrazolo[4,3-c]pyridin-7-yl)-2-oxo-2-[rac-(2S)-2-[3-(diethylamino)phenyl]-1-piperidyl]acetamide (0.01712 g, 39.31 μmol, 33.70% yield) and Compound 276-N-(6-amino-3-pyridyl)-2-[(2S,5R)-5-methyl-2-[2-(trifluoromethyl)-1,3-benzothiazol-5-yl]-1-piperidyl]-2-oxo-acetamide (0.05775 g, 124.60 μmol, 50.22% yield).

### Compound 236:

Preparative: RT (Column: CHIRALCEL OJ-H (250×20 mm, 5 mkm); Mobile Phase: Hexane:IPA:MeOH:DEA, 70:15:15:0.05; Flow Rate: 12 mL/min)=17.180 min.

Analytical: RT (Column: Chiralcel OJ-H (250×4.6 mm, 5 mkm)-1; Mobile Phase: Hexane (0.1% EDA):IPA:MeOH, 70:15:15; Flow Rate: 0.6 mL/min)=16.905 min.

¹H NMR (600 MHz, dmso) δ 1.01-1.08 (m, 6H), 1.15-1.49 (m, 2H), 1.47-1.69 (m, 3H), 1.73-1.95 (m, 1H), 2.38-2.47 (m, 2H), 2.68-3.10 (m, 1H), 3.15-3.28 (m, 2H), 3.59-4.38 (m, 1H), 5.16-5.71 (m, 1H), 6.32-6.62 (m, 3H), 6.62-6.91 (m, 2H), 7.06-7.18 (m, 1H), 7.49-7.79 (m, 1H), 8.07-8.30 (m, 1H), 9.53-10.57 (m, 1H), 12.58-13.43 (m, 1H).

LCMS(ESI): [M+H]⁺ m/z: calcd 436.2; found 436.2; Rt=1.375 min.

### Compound 276:

Preparative: RT (Column: CHIRALCEL OJ-H (250×20 mm, 5 mkm); Mobile Phase: Hexane: IPA:MeOH:DEA, 70:15:15:0.05; Flow Rate: 12 mL/min)=25.583 min.

Analytical: RT (Column: Chiralcel OJ-H (250×4.6 mm, 5 mkm)-1; Mobile Phase: Hexane (0.1% EDA):IPA:MeOH, 70:15:15; Flow Rate: 0.6 mL/min)=29.567 min.

¹H NMR (600 MHz, dmso) δ 0.99-1.07 (m, 6H), 1.13-1.47 (m, 2H), 1.47-1.70 (m, 3H), 1.75-1.97 (m, 1H), 2.36-2.46 (m, 2H), 2.69-3.11 (m, 1H), 3.17-3.28 (m, 2H), 3.59-4.34 (m, 1H), 5.17-5.70 (m, 1H), 6.35-6.58 (m, 3H), 6.62-6.89 (m, 2H), 7.08-7.18 (m, 1H), 7.46-7.79 (m, 1H), 8.09-8.20 (m, 1H), 9.57-10.73 (m, 1H), 12.51-13.52 (m, 1H).

LCMS(ESI): [M+H]⁺ m/z: calcd 436.2; found 436.2; Rt=1.375 min.

### Example 211. N-(4-Amino-1H-pyrazolo[4,3-c]pyridin-7-yl)-2-oxo-2-[rac-(2R,5S)-2-(2,3-dihydrobenzofuran-4-yl)-5-methyl-1-piperidyl]acetamide (Compound 238)

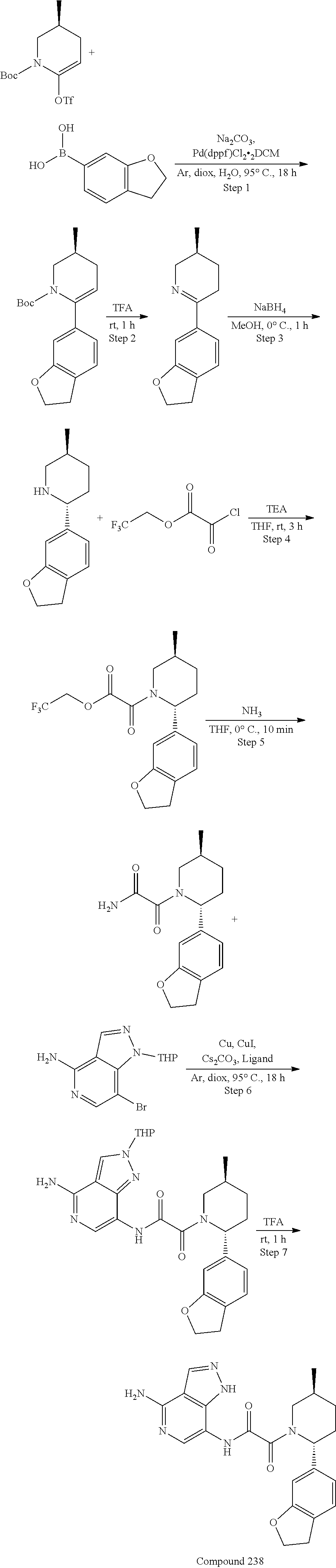

### Step 1: The Synthesis of tert-Butyl rac-(3S)-6-(2,3-dihydrobenzofuran-6-yl)-3-methyl-3,4-dihydro-2H-pyridine-1-carboxylate

To a solution of tert-butyl rac-(3S)-3-methyl-6-(trifluoromethylsulfonyloxy)-3,4-dihydro-2H-pyridine-1-carboxylate (5.05 g, 14.64 mmol), 2,3-dihydrobenzofuran-6-ylboronic acid (2 g, 12.20 mmol), Sodium carbonate (3.88 g, 36.59 mmol, 1.53 mL) in Dioxane (60 mL) and Water (15 mL), Pd(dppf)Cl₂·CH₂Cl₂ (597.66 mg, 731.86 μmol) was added under Argon atmosphere. The resulting mixture was stirred at 95° C. for 18 hr and cooled. The organic layer was separated, evaporated, the residue was dissolved in MTBE (500 ml), dried over Na₂SO₄, filtered and evaporated in vacuo to give tert-butyl rac-(3S)-6-(2,3-dihydrobenzofuran-6-yl)-3-methyl-3,4-dihydro-2H-pyridine-1-carboxylate (3.5 g, crude).

Crude substance was used for the next step without further purification.

LCMS(ESI): [M-Boc]⁺ m/z: calcd 216.2; found 216.0; Rt=1.362 min.

### Step 2: The Synthesis of rac-(3S)-6-(2,3-Dihydrobenzofuran-6-yl)-3-methyl-2,3,4,5-tetrahydropyridine

tert-Butyl rac-(3S)-6-(2,3-dihydrobenzofuran-6-yl)-3-methyl-3,4-dihydro-2H-pyridine-1-carboxylate (3.5 g, 11.10 mmol) was taken up with Trifluoroacetic acid (20 g, 175.40 mmol, 13.42 mL). The reaction mixture was stirred at 25° C. for 1 hr, evaporated and diluted with water (100 mL). The aqueous solution was washed with MTBE (2*50 mL), pH of the solution was adjusted to 8-9 with NaOH and extracted with DCM (3*50 mL). Combined organic layer was dried over Na₂SO₄ and evaporated in vacuo to give rac-(3S)-6-(2,3-dihydrobenzofuran-6-yl)-3-methyl-2,3,4,5-tetrahydropyridine (0.9 g, 4.18 mmol, 37.67% yield).

LCMS(ESI): [M+H]⁺ m/z: calcd 216.2; found 216.2; Rt=0.760 min.

### Step 3: The Synthesis of rac-(2R,5S)-2-(2,3-Dihydrobenzofuran-6-yl)-5-methyl-piperidine

NaBH₄ (158.15 mg, 4.18 mmol, 147.26 μL) was added in portions to a stirred solution of rac-(3S)-6-(2,3-dihydrobenzofuran-6-yl)-3-methyl-2,3,4,5-tetrahydropyridine (0.9 g, 4.18 mmol) in MeOH (20 mL) at 0° C. The resulting mixture was stirred at 0° C. for 1 hr, and then evaporated in vacuo. The residue was diluted with brine (5 mL) and extracted with dichloromethane (2*20 mL). The combined organic extracts were dried over sodium sulphate and evaporated in vacuo to afford rac-(2R,5S)-2-(2,3-dihydrobenzofuran-6-yl)-5-methyl-piperidine (0.8 g, 3.68 mmol, 88.06% yield).

LCMS(ESI): [M+H]⁺ m/z: calcd 218.0; found 218.0; Rt=0.696 min.

### Step 4: The Synthesis of 2,2,2-Trifluoroethyl 2-oxo-2-[rac-(2R,5S)-2-(2,3-dihydrobenzofuran-6-yl)-5-methyl-1-piperidyl]acetate

To a solution of rac-(2R,5S)-2-(2,3-dihydrobenzofuran-6-yl)-5-methyl-piperidine (0.8 g, 3.68 mmol) and TEA (1.86 g, 18.41 mmol, 2.57 mL) in THF (40 mL), 2,2,2-trifluoroethyl 2-chloro-2-oxo-acetate (1.75 g, 9.20 mmol) was added portionwise at 0° C. The resulting mixture was stirred for 3 hr. LCMS showed full conversion of SM. The reaction mixture was directly used for the next step.

LCMS(ESI): [M+H]⁺ m/z: calcd 372.0; found 372.0; Rt=1.283 min.

### Step 5: The Synthesis of 2-Oxo-2-[rac-(2R,5S)-2-(2,3-dihydrobenzofuran-6-yl)-5-methyl-1-piperidyl]acetamide

Through a solution of 2,2,2-trifluoroethyl 2-oxo-2-[rac-(2R,5S)-2-(2,3-dihydrobenzofuran-6-yl)-5-methyl-1-piperidyl]acetate (1.3 g, 3.50 mmol) in THF (50 mL), ammonia was bubbled during 10 min at 0° C. The formed precipitate was filtered off, washed with THF (30 mL) and the solvent was evaporated in vacuo to give crude product (1.5 g), which was purified by gradient chromatography (CHCl₃-ACN) affording 2-oxo-2-[rac-(2R,5S)-2-(2,3-dihydrobenzofuran-6-yl)-5-methyl-1-piperidyl]acetamide (0.4 g, 1.39 mmol, 39.63% yield).

LCMS(ESI): [M+H]⁺ m/z: calcd 289.2; found 289.2; Rt=0.948 min.

### Step 6: The Synthesis of N-(4-Amino-2-tetrahydropyran-2-yl-pyrazolo[4,3-c]pyridin-7-yl)-2-oxo-2-[rac-(2R,5S)-2-(2,3-dihydrobenzofuran-6-yl)-5-methyl-1-piperidyl]acetamide

To a mixture of 2-oxo-2-[rac-(2R,5S)-2-(2,3-dihydrobenzofuran-6-yl)-5-methyl-1-piperidyl]acetamide (150 mg, 520.22 μmol), 7-bromo-2-tetrahydropyran-2-yl-pyrazolo[4,3-c]pyridin-4-amine (200.96 mg, 676.28 μmol), Copper (6.61 mg, 104.04 μmol), Copper (I) iodide (59.45 mg, 312.13 μmol, 10.58 μL), Cesium carbonate (203.40 mg, 624.26 μmol) and rac-(1R,2R)—N1,N2-dimethylcyclohexane-1,2-diamine (44.40 mg, 312.13 μmol), Dioxane (5 mL) was added. The resulting mixture was evacuated, refiled with Argon three time, heated at 95° C. for 18 hr and cooled. The inorganic precipitate was filtered off and washed with DCM (50 mL) to give N-(4-amino-2-tetrahydropyran-2-yl-pyrazolo[4,3-c]pyridin-7-yl)-2-oxo-2-[rac-(2R,5S)-2-(2,3-dihydrobenzofuran-6-yl)-5-methyl-1-piperidyl]acetamide (300 mg, crude).

This substance was used for the next step without further purification.

LCMS(ESI): [M+H]⁺ m/z: calcd 505.2; found 505.4; Rt=1.008 min.

### Step 7: The Synthesis of N-(4-Amino-1H-pyrazolo[4,3-c]pyridin-7-yl)-2-oxo-2-[rac-(2R,5S)-2-(2,3-dihydrobenzofuran-4-yl)-5-methyl-1-piperidyl]acetamide (Compound 238)

N-(4-Amino-2-tetrahydropyran-2-yl-pyrazolo[4,3-c]pyridin-7-yl)-2-oxo-2-[rac-(2R,5S)-2-(2,3-dihydrobenzofuran-6-yl)-5-methyl-1-piperidyl]acetamide (0.29 g, 574.74 μmol) was dissolved in Trifluoroacetic acid (8 g, 70.16 mmol, 5.37 mL), stirred at 25° C. for 1 hr and evaporated in vacuo to give crude product (0.5 g), which was purified by HPLC:
1. Device (Mobile Phase, Column): SYSTEM 20-20-65% 0-1-5 min H₂O/ACN/0.1% NH₄OH, flow: 30 mL/min (loading pump 4 mL/min acetonitrile); column: XBridge BEH C18 100×19 mm, 5 um
2. Device (Mobile Phase, Column): SYSTEM 30-30-65% 0-1-5 min H₂O/MeOH/0.1% NH₄OH, flow: 30 mL/min (loading pump 4 mL/min methanol); column: XBridge BEH C18 100×19 mm, 5 um
3. Device (Mobile Phase, Column): SYSTEM 5-5-45% 0-1-5 min H₂O/ACN/0.2% FA, flow: 30 mL/min (loading pump 4 mL/min acetonitrile); column: Chromatorex 18 SMB100-5T 100×19 mm 5 um
to obtain N-(4-amino-1H-pyrazolo[4,3-c]pyridin-7-yl)-2-oxo-2-[rac-(2R,5S)-2-(2,3-dihydrobenzofuran-6-yl)-5-methyl-1-piperidyl]acetamide (27 mg, 57.88 μmol, 10.07% yield, HCO₂H).

¹H NMR (600 MHz, DMSO-d₆) δ 0.10-1.06 (m, 3H), 1.18-2.33 (m, 5H), 2.73-3.28 (m, 3H), 3.68-4.26 (m, 1H), 4.40-4.60 (m, 2H), 4.87-5.70 (m, 1H), 6.39-6.95 (m, 4H), 7.11-7.26 (m, 1H), 7.47-7.77 (m, 1H), 8.14-8.26 (m, 1H), 9.52-10.63 (m, 1H), 12.42-13.51 (m, 1H).

LCMS(ESI): [M+H]⁺ m/z: calcd 421.2; found 421.2; Rt=2.539 min.

### Example 212. N-(4-Amino-1H-pyrazolo[4,3-c]pyridin-7-yl)-2-oxo-2-[rac-(2S,5R)-5-methyl-2-oxazol-5-yl-1-piperidyl]acetamide (Compound 239)

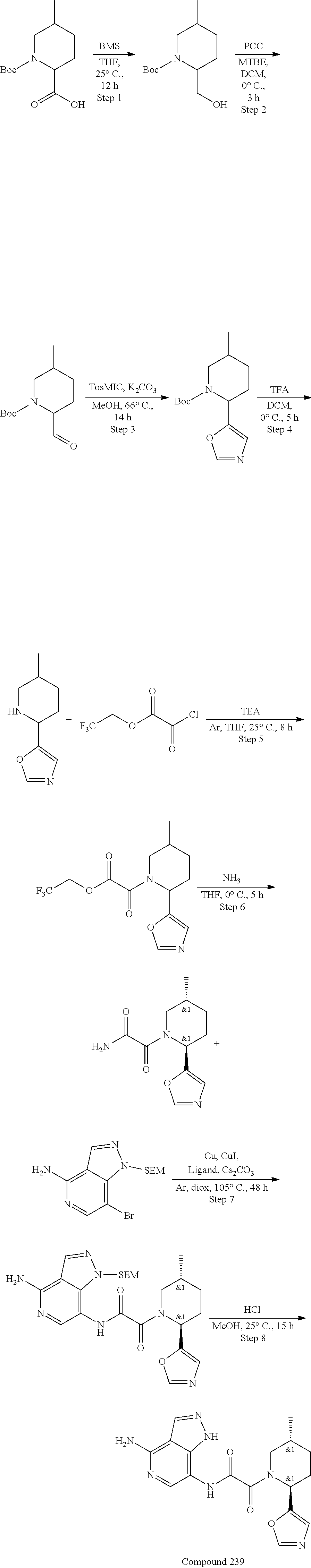

### Step 1: The Synthesis of tert-Butyl rac-(2S,5R)-2-(hydroxymethyl)-5-methyl-piperidine-1-carboxylate

To a stirred at 0° C. solution of rac-(2S,5R)-1-tert-butoxycarbonyl-5-methyl-piperidine-2-carboxylic acid (3 g, 12.33 mmol) in THF (101.75 mL), Borane dimethyl sulfide complex (1.41 g, 18.50 mmol, 1.75 mL) was added dropwise. The resulting mixture was stirred at 25° C. for 12 hr, and methanol (20 ml) was added dropwise. The resulting mixture was evaporated, taken up with water (50 mL) and extracted with DCM (3*50 mL). The combined organic layer was washed with brine (50 mL), dried over Na₂SO₄ and evaporated in vacuo to give tert-butyl rac-(2S,5R)-2-(hydroxymethyl)-5-methyl-piperidine-1-carboxylate (2.8 g, 12.21 mmol, 99.02% yield).

LCMS(ESI): [M−tBu]⁺ m/z: calcd 174.2; found 174.2; Rt=1.003 min.

### Step 2: The Synthesis of tert-Butyl 2-formyl-5-methyl-piperidine-1-carboxylate

To the stirring solution of tert-butyl 2-(hydroxymethyl)-5-methyl-piperidine-1-carboxylate (0.3 g, 1.31 mmol) in MTBE (100 mL) was added portions PCC (564.00 mg, 2.62 mmol) and the resulting mixture was stirred at 0° C. for 3 hr. The reaction mixture poured in MTBE (100 mL) and filtered through a pad of silica gel and concentrated in vacuo to give tert-butyl 2-formyl-5-methyl-piperidine-1-carboxylate (0.15 g, 659.92 μmol, 50.44% yield).

¹H NMR (400 MHZ, CDCl₃) δ (ppm) 0.94 (d, 3H), 1.16 (m, 3H), 1.46 (m, 9H), 1.60 (m, 2H), 3.19 (m, 1H), 3.41 (m, 1H), 4.02 (m, 1H), 9.48 (s, 1H).

### Step 3: The Synthesis of tert-Butyl 5-methyl-2-oxazol-5-yl-piperidine-1-carboxylate

To a solution of tert-butyl 2-formyl-5-methyl-piperidine-1-carboxylate (1.3 g, 5.72 mmol) and Potassium carbonate-granular (1.58 g, 11.44 mmol, 690.34 μL) in MeOH (20 mL) was added TosMIC (1.34 g, 6.86 mmol) at r.t. under argon. The reaction mixture was then stirred for 14 hr at 66° C. The resulting mixture was evaporated, taken up with water (60 mL) and extracted with MTBE (40 mL). The combined organic layer was washed with brine (20 mL), dried over Na₂SO₄ and evaporated in vacuo to give tert-butyl 5-methyl-2-oxazol-5-yl-piperidine-1-carboxylate (0.5 g, 1.88 mmol, 32.82% yield).

LCMS(ESI): [M+H]⁺ m/z: calcd 267.4; found 267.4; Rt=3.722 min.

### Step 4: The Synthesis of 5-(5-Methyl-2-piperidyl) oxazole

Trifluoroacetic acid (256.86 mg, 2.25 mmol, 173.56 μL) was added to the solution of tert-butyl 5-methyl-2-oxazol-5-yl-piperidine-1-carboxylate (0.6 g, 2.25 mmol) in Dichloromethane. Resulting mixture was stirred at 0° C. for 5 hr. Then, volatiles were removed under reduced pressure and residue was partitioned between 20% aq. K₂CO₃ solution (30 mL) and ethyl acetate (50 mL). Organic layer was separated, dried over Na₂SO₄ and concentrated in vacuo, affording 5-(5-methyl-2-piperidyl) oxazole (0.15 g, 902.42 μmol, 40.06% yield).

LCMS(ESI): [M+2H]⁺ m/z: calcd 168.2; found 168.2; Rt=0.788 min.

### Step 5: The Synthesis of 2,2,2-Trifluoroethyl 2-(5-methyl-2-oxazol-5-yl-1-piperidyl)-2-oxo-acetate

To a solution of 5-(5-methyl-2-piperidyl) oxazole (0.15 g, 902.42 μmol) and TEA (136.97 mg, 1.35 mmol, 188.67 μL) in THF (14.81 mL) was added 2,2,2-trifluoroethyl 2-chloro-2-oxo-acetate (206.30 mg, 1.08 mmol) dropwise at 0° C. under argon. The reaction mixture was then stirred for 8 hr at r.t., then evaporated in vacuo to give 2,2,2-trifluoroethyl 2-(5-methyl-2-oxazol-5-yl-1-piperidyl)-2-oxo-acetate (0.17 g, 530.81 μmol, 58.82% yield).

LCMS(ESI): [M+H]⁺ m/z: calcd 321.2; found 321.2; Rt=3.252 min.

### Step 6: The Synthesis of 2-(5-Methyl-2-oxazol-5-yl-1-piperidyl)-2-oxo-acetamide

2,2,2-Trifluoroethyl 2-(5-methyl-2-oxazol-5-yl-1-piperidyl)-2-oxo-acetate (0.6 g, 1.87 mmol) was dissolved in THF (30 mL) and was blow ammonium (675.94 mg, 39.69 mmol). Resulting solution was stirred at 0° C. for 5 hr. The resulting mixture was evaporated in vacuo and residue was triturated with THF (20 mL*2), filtered and combined organic was evaporated in vacuo 2-(5-methyl-2-oxazol-5-yl-1-piperidyl)-2-oxo-acetamide (0.34 g, 1.43 mmol, 76.49% yield).

LCMS(ESI): [M+H]⁺ m/z: calcd 238.2; found 238.2; Rt=1.985 min.

### Step 7: The Synthesis of N-[4-Amino-1-(2-trimethylsilylethoxymethyl)pyrazolo[4,3-c]pyridin-7-yl]-2-oxo-2-[rac-(2S,5R)-5-methyl-2-oxazol-5-yl-1-piperidyl]acetamide

A mixture of 2-oxo-2-[rac-(2S,5R)-5-methyl-2-oxazol-5-yl-1-piperidyl]acetamide (280 mg, 1.18 mmol), 7-bromo-1-(2-trimethylsilylethoxymethyl)pyrazolo[4,3-c]pyridin-4-amine (445.66 mg, 1.30 mmol), copper (4.46 mg, 70.12 μmol), Copper (I) iodide (120 mg, 630.09 μmol, 21.35 μL), caesium carbonate (600 mg, 1.84 mmol) and rac-(1R,2R)—N1,N2-dimethylcyclohexane-1,2-diamine (120 mg, 843.64 μmol) in 1,4-dioxane (6 mL) was stirred in a sealed vial under argon at 105° C. for 48 hr. The resulting mixture was cooled down, diluted with DMSO (1 mL) and submitted to reverse phase HPLC (column: XBridge C18 OBD 100×19 mm 5 um; mobile phase: 45-80% 0-5 min H₂O/ACN/0.1% NH₄OH; flow rate: 30 mL/min (loading pump 4 mL/min acetonitrile) to afford N-[4-amino-1-(2-trimethylsilylethoxymethyl)pyrazolo[4,3-c]pyridin-7-yl]-2-oxo-2-[rac-(2S,5R)-5-methyl-2-oxazol-5-yl-1-piperidyl]acetamide (90 mg, 180.13 μmol, 15.26% yield) as light-brown gum, which was used in the next step.

LCMS(ESI): [M+H]⁺ m/z: calcd 500.2; found 500.2; Rt=3.225 min.

### Step 8: The Synthesis of N-(4-Amino-1H-pyrazolo[4,3-c]pyridin-7-yl)-2-oxo-2-[rac-(2S,5R)-5-methyl-2-oxazol-5-yl-1-piperidyl]acetamide (Compound 239)

Hydrogen chloride solution 4.0M in dioxane (3.15 g, 12.01 mmol, 3.00 mL, 13.9% purity) was added to a stirred solution of N-[4-amino-1-(2-trimethylsilylethoxymethyl)pyrazolo[4,3-c]pyridin-7-yl]-2-oxo-2-[rac-(2S,5R)-5-methyl-2-oxazol-5-yl-1-piperidyl]acetamide (90 mg, 180.13 μmol) in methanol (3 mL) at 25° C. The resulting solution was stirred at 25° C. for 15 hr, then concentrated to dryness in vacuo and the residue was submitted to reverse phase HPLC (column: Chromatorex 18 SMB100-5T 100×19 mm 5 um; mobile phase: 0-0-30% 0-1-6 min H₂O/ACN/0.1% FA, flow: 30 mL/min (loading pump 4 mL/min acetonitrile)) to afford Compound 239 N-(4-amino-1H-pyrazolo[4,3-c]pyridin-7-yl)-2-oxo-2-[rac-(2S,5R)-5-methyl-2-oxazol-5-yl-1-piperidyl]acetamide (40 mg, 96.29 μmol, 53.46% yield, HCOOH) as light-yellow solid. Attempts to separate cis/trans-isomers by chiral HPLC were unsuccessful.

¹H NMR (600 MHz, dmso) δ 0.65-1.01 (m, 3H), 1.20-1.38 (m, 1H), 1.55-1.73 (m, 1H), 1.73-1.90 (m, 1H), 1.90-2.04 (m, 1H), 2.14-2.26 (m, 1H), 2.60-2.95 (m, 1H), 3.54-3.89 (m, 1H), 3.91-4.61 (m, 1H), 4.94-5.82 (m, 1H), 6.76-7.14 (m, 2H), 7.15-7.23 (m, 1H), 7.36-7.77 (m, 1H), 8.14-8.30 (m, 1H), 8.31-8.39 (m, 1H), 9.57-10.67 (m, 1H), 12.61-13.63 (m, 1H).

LCMS(ESI): [M+H]⁺ m/z: calcd 370.2; found 370.2; Rt=1.622 min.

### Example 213. N-(4-Amino-1H-pyrazolo[4,3-c]pyridin-7-yl)-2-oxo-2-[rac-(2R,5S)-2-(2-isopropylindazol-6-yl)-5-methyl-1-piperidyl]acetamide (Compound 240)

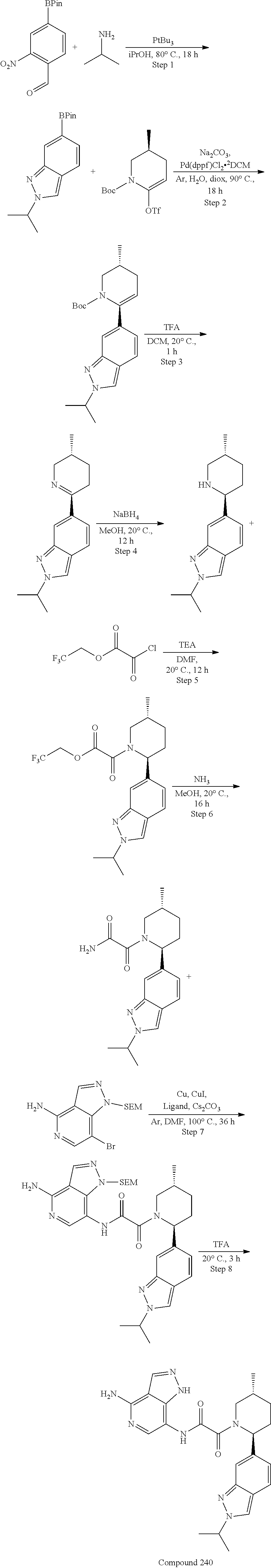

### Step 1: The Synthesis of 2-Isopropyl-6-(4,4,5,5-tetramethyl-1,3,2-dioxaborolan-2-yl)indazole

2-Nitro-4-(4,4,5,5-tetramethyl-1,3,2-dioxaborolan-2-yl)benzaldehyde (5 g, 18.05 mmol) and propan-2-amine (2.13 g, 36.09 mmol, 3.09 mL) were dissolved in i-PrOH (50 mL) and the resulting solution was heated at 80° C. for 2 hr in a sealed tube. The reaction mixture was cooled to RT and Tri-n-butylphosphine, 95% (10.95 g, 54.14 mmol, 13.36 mL) was added, then the resulting mixture was heated at 80° C. overnight in a sealed tube. The reaction mixture was concentrated on vacuo. The crude product was purified by FCC (MTBE in hexanes from 16% to 50%). The reaction is successful. 2-Isopropyl-6-(4,4,5,5-tetramethyl-1,3,2-dioxaborolan-2-yl)indazole (1.3 g, 4.54 mmol, 25.17% yield) was obtained as a brown gum.

LCMS(ESI): [M+H]⁺ m/z: calcd 287.2; found 287.2; Rt=1.380 min.

### Step 2: The Synthesis of tert-Butyl rac-(3S)-6-(2-isopropylindazol-6-yl)-3-methyl-3,4-dihydro-2H-pyridine-1-carboxylate

2-Isopropyl-6-(4,4,5,5-tetramethyl-1,3,2-dioxaborolan-2-yl)indazole (1.3 g, 4.54 mmol), tert-butyl rac-(3S)-3-methyl-6-(trifluoromethylsulfonyloxy)-3,4-dihydro-2H-pyridine-1-carboxylate (1.73 g, 5.00 mmol), Sodium carbonate (962.94 mg, 9.09 mmol, 380.31 μL) and Pd(dppf)Cl2·DCM (185.49 mg, 227.13 μmol) were mixed in dioxane (10 mL)+water (3 mL) under argon, and then stirred overnight at 90° C. for 12 hr in vial. The solid was filtered off, the filtrate was diluted with EA, the organic layer was separated and the aqueous layer was extracted with additional EA, the combined organic layer was washed with brine, dried over Na₂SO₄, filtered and concentrated on vacuo. The obtained product was used in the next step without further purification. The reaction was successful. tert-Butyl rac-(3S)-6-(2-isopropylindazol-6-yl)-3-methyl-3,4-dihydro-2H-pyridine-1-carboxylate (2.51 g, crude) was obtained as a red gum.

LCMS(ESI): [M+H]⁺ m/z: calcd 354.2; found 354.2; Rt=1.297 min.

### Step 3: The Synthesis of 2-Isopropyl-6-[rac-(3S)-3-methyl-2,3,4,5-tetrahydropyridin-6-yl]indazole

tert-Butyl rac-(3S)-6-(2-isopropylindazol-6-yl)-3-methyl-3,4-dihydro-2H-pyridine-1-carboxylate (2.51 g, 7.06 mmol) was dissolved in a mixture of DCM (10 mL) and TFA (10 mL). The resulting clear solution was stirred for 1 hr at 20° C. The reaction mixture was concentrated on vacuo. The obtained residue was dissolved in water and extracted with MTBE (3 times), after that the aqueous layer was basified with NaOH and extracted with DCM (3 times), combined DCM layers was dried over Na₂SO₄, filtered and evaporated. The reaction is successful. 2-Isopropyl-6-[rac-(3S)-3-methyl-2,3,4,5-tetrahydropyridin-6-yl]indazole (0.35 g, 1.37 mmol, 19.41% yield) was obtained as a brown solid.

LCMS(ESI): [M+H]⁺ m/z: calcd 256.2; found 256.2; Rt=0.752 min.

### Step 4: The Synthesis of 2-Isopropyl-6-[rac-(2R,5S)-5-methyl-2-piperidyl]indazole

To a stirring suspension of 2-isopropyl-6-[rac-(3S)-3-methyl-2,3,4,5-tetrahydropyridin-6-yl]indazole (0.35 g, 1.37 mmol) in MeOH (8 mL) was added Sodium Borohydride (103.71 mg, 2.74 mmol, 96.56 μL) in portions. Resulting mixture was stirred at 20° C. for 12 hr. The solvent was removed under reduced pressure and residue was partitioned between water and DCM. The organic layer was separated, dried over Na₂SO₄ and concentrated in vacuo. The obtained product was used in the next step without further purification. The reaction is successful. 2-Isopropyl-6-[rac-(2R,5S)-5-methyl-2-piperidyl]indazole (0.3 g, 1.17 mmol, 85.04% yield) was obtained as a brown gum.

LCMS(ESI): [M+H]⁺ m/z: calcd 258.2; found 258.4; Rt=0.641 min.

### Step 5: The Synthesis of 2,2,2-Trifluoroethyl 2-oxo-2-[rac-(2R,5S)-2-(2-isopropylindazol-6-yl)-5-methyl-1-piperidyl]acetate

2,2,2-Trifluoroethyl 2-chloro-2-oxo-acetate (244.26 mg, 1.28 mmol, 232.63 μL) was added dropwise to a solution of 2-isopropyl-6-[rac-(2R,5S)-5-methyl-2-piperidyl]indazole (0.3 g, 1.17 mmol) and TEA (135.64 mg, 1.34 mmol, 186.83 μL) in DCM (10.77 mL) at 0° C. After addition was complete, cooling bath was removed and resulting mixture was allowed to warm up to 20° C. and stirred for 12 hr. The reaction mixture was washed with Na₂CO₃, brine, dried over Na₂SO₄ and concentrated under reduced pressure. The obtained product was used in the next step without further purification. The reaction is successful. 2,2,2-Trifluoroethyl 2-oxo-2-[rac-(2R,5S)-2-(2-isopropylindazol-6-yl)-5-methyl-1-piperidyl]acetate (0.34 g, 826.41 μmol, 70.90% yield) was obtained as a brown gum.

LCMS(ESI): [M+H]⁺ m/z: calcd 412.2; found 412.2; Rt=1.216 min.

### Step 6: The Synthesis of 2-Oxo-2-[rac-(2R,5S)-2-(2-isopropylindazol-6-yl)-5-methyl-1-piperidyl]acetamide

2,2,2-trifluoroethyl 2-oxo-2-[rac-(2R,5S)-2-(2-isopropylindazol-6-yl)-5-methyl-1-piperidyl]acetate (0.34 g, 826.41 μmol) was dissolved in MeOH(NH₃) (10 mL) as stirred overnight at 20° C. The reaction mixture was evaporated to dryness. The obtained product was used in the next step without further purification. The reaction is successful. 2-Oxo-2-[rac-(2R,5S)-2-(2-isopropylindazol-6-yl)-5-methyl-1-piperidyl]acetamide (0.18 g, 548.10 μmol, 66.32% yield) was obtained as a brown gum.

LCMS(ESI): [M+H]⁺ m/z: calcd 329.1; found 329.1; Rt=1.172 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-2-(2-isopropylindazol-6-yl)-5-methyl-1-piperidyl]acetamide

2-Oxo-2-[rac-(2R,5S)-2-(2-isopropylindazol-6-yl)-5-methyl-1-piperidyl]acetamide (0.18 g, 548.10 μmol), 7-bromo-1-(2-trimethylsilylethoxymethyl)pyrazolo[4,3-c]pyridin-4-amine (225.79 mg, 657.72 μmol), Copper (I) iodide (20.88 mg, 109.62 μmol, 3.71 μL), Cesium carbonate (357.16 mg, 1.10 mmol) and (1R,2R)—N1,N2-dimethylcyclohexane-1,2-diamine (93.55 mg, 657.72 μmol) were mixed in DMF (5 mL) under argon, and then stirred overnight at 100° C. for 36 hr in vial. The reaction mixture was submitted to HPLC (2-10 min 30-60% water-MeCN+FA; flow: 30 mL/min; loading pump 4 mL/min MeCN+FA; column SunFire 19*100 mm). The reaction was successful. N-[4-Amino-1-(2-trimethylsilylethoxymethyl)pyrazolo[4,3-c]pyridin-7-yl]-2-oxo-2-[rac-(2R,5S)-2-(2-isopropylindazol-6-yl)-5-methyl-1-piperidyl]acetamide (0.021 g, 35.55 μmol, 6.49% yield) was obtained as a brown solid.

LCMS(ESI): [M+H]⁺ m/z: calcd 591.2; found 591.2; Rt=1.396 min.

### Step 8: The Synthesis of N-(4-Amino-1H-pyrazolo[4,3-c]pyridin-7-yl)-2-oxo-2-[rac-(2R,5S)-2-(2-isopropylindazol-6-yl)-5-methyl-1-piperidyl]acetamide (Compound 240)

N-[4-Amino-1-(2-trimethylsilylethoxymethyl)pyrazolo[4,3-c]pyridin-7-yl]-2-oxo-2-[rac-(2R,5S)-2-(2-isopropylindazol-6-yl)-5-methyl-1-piperidyl]acetamide (0.021 g, 35.55 μmol) was dissolved in TFA (121.59 mg, 1.07 mmol, 82.16 μL) and stirred at 20° C. for 3 hr. The reaction mixture was submitted to HPLC (2-10 min 0-90% water-methanol+FA; flow: 30 mL/min; loading pump 4 mL/min methanol+FA column SunFire 19*100 mm). The reaction is successful. N-(4-Amino-1H-pyrazolo[4,3-c]pyridin-7-yl)-2-oxo-2-[rac-(2R,5S)-2-(2-isopropylindazol-6-yl)-5-methyl-1-piperidyl]acetamide (0.005 g, 9.87 μmol, 27.77% yield, HCOOH) was obtained as a light-yellow solid.

¹H NMR (600 MHz, dmso) δ 0.92-1.08 (m, 3H), 1.28-1.43 (m, 1H), 1.45-1.51 (m, 1H), 1.51-1.56 (m, 6H), 1.63-1.86 (m, 1H), 1.87-2.14 (m, 1H), 2.17-2.35 (m, 1H), 2.79-3.14 (m, 1H), 3.44-4.16 (m, 1H), 4.73-5.11 (m, 1H), 5.34-5.73 (m, 1H), 6.62-6.94 (m, 2H), 6.94-7.12 (m, 1H), 7.54-7.59 (m, 1H), 7.63-7.69 (m, 1H), 7.72-8.13 (m, 1H), 8.14-8.22 (m, 1H), 8.32-8.39 (m, 1H), 9.41-10.73 (m, 1H), 12.68-13.30 (m, 1H).

LCMS(ESI): [M+H]⁺ m/z: calcd 461.2; found 461.2; Rt=0.705 min.

### Example 214. N-(4-Amino-1H-pyrazolo[4,3-c]pyridin-7-yl)-2-oxo-2-[rac-(2R,5S)-2-(5,6-dimethyl-3-pyridyl)-5-methyl-1-piperidyl]acetamide (Compound 241)

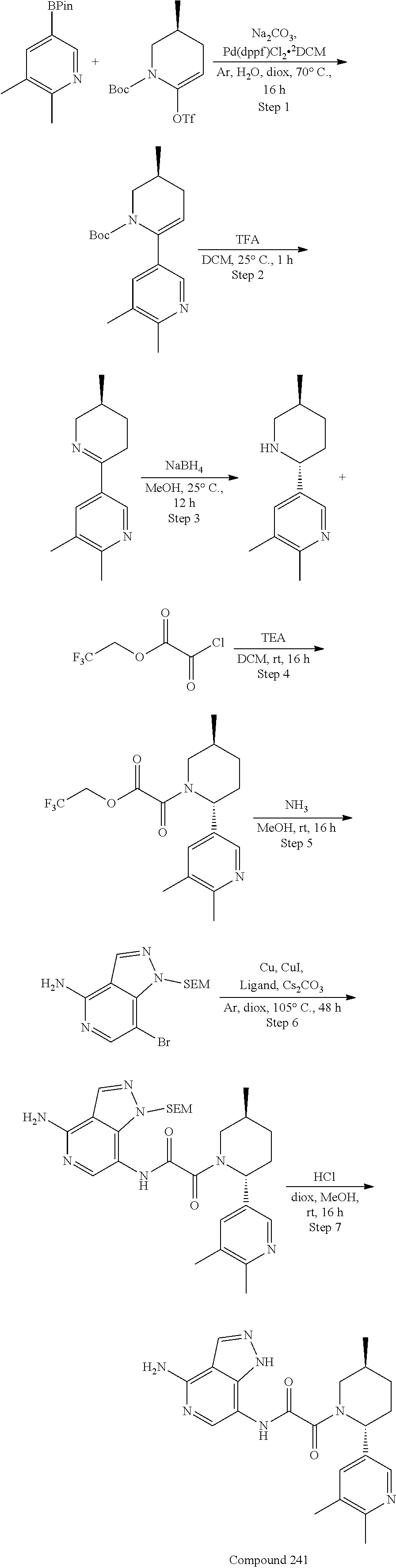

### Step 1: The Synthesis of tert-Butyl rac-(3S)-6-(5,6-dimethyl-3-pyridyl)-3-methyl-3,4-dihydro-2H-pyridine-1-carboxylate

2,3-Dimethyl-5-(4,4,5,5-tetramethyl-1,3,2-dioxaborolan-2-yl)pyridine (3 g, 12.87 mmol), tert-butyl rac-(3S)-3-methyl-6-(trifluoromethylsulfonyloxy)-3,4-dihydro-2H-pyridine-1-carboxylate (4.44 g, 12.87 mmol) and Sodium carbonate (4.09 g, 38.61 mmol, 1.62 mL) was added to a mixture of dioxane (29.32 mL) and water (9.77 mL). The resulting mixture was evacuated and then backfilled with argon, this operation was repeated three times, then Pd(dppf)Cl₂·DCM (525.48 mg, 643.46 μmol) was added under argon. The reaction mixture was stirred under argon at 70° C. for 16 hr, then cooled and filtered. The filtercake was washed with 1,4-dioxane (2*20 mL) and discarded. The filtrate was evaporated in vacuo to afford tert-butyl rac-(3S)-6-(5,6-dimethyl-3-pyridyl)-3-methyl-3,4-dihydro-2H-pyridine-1-carboxylate (3.8 g, 12.57 mmol, 97.64% yield).

LCMS(ESI): [M+H]⁺ m/z: calcd 303.2; found 303.2; Rt=1.122 min.

### Step 2: The Synthesis of 2,3-Dimethyl-5-[rac-(3S)-3-methyl-2,3,4,5-tetrahydropyridin-6-yl]pyridine

The solution of tert-butyl rac-(3S)-6-(5,6-dimethyl-3-pyridyl)-3-methyl-3,4-dihydro-2H-pyridine-1-carboxylate (3.8 g, 12.57 mmol) in DCM (25.48 mL) with TFA (21.49 g, 188.49 mmol, 14.52 mL) was stirred at 25° C. for 1 hr, and then evaporated in vacuo. Water (50 mL) was added to the residue and filtered off, then pH was adjusted to 10 with a 10% aqueous solution of sodium hydroxide. The resulting mixture was extracted with dichloromethane (2*30 mL). The combined organic extracts were dried over sodium sulphate and evaporated in vacuo to afford 2,3-dimethyl-5-[rac-(3S)-3-methyl-2,3,4,5-tetrahydropyridin-6-yl]pyridine (1.6 g, 7.91 mmol, 62.94% yield) as brown oil, which was used directly in the next step.

LCMS(ESI): [M+H]⁺ m/z: calcd 203.2; found 203.2; Rt=0.460 min.

### Step 3: The Synthesis of 2,3-Dimethyl-5-[rac-(2R,5S)-5-methyl-2-piperidyl]pyridine

To a stirred solution of 2,3-dimethyl-5-[rac-(3S)-3-methyl-2,3,4,5-tetrahydropyridin-6-yl]pyridine (1.6 g, 7.91 mmol) in MeOH (19.44 mL) was added Sodium Borohydride (598.46 mg, 15.82 mmol, 557.22 μL) at 0° C. The resulting reaction mixture was stirred at 25° C. for 12 hr. Upon completion, the reaction mixture was concentrated under reduced pressure, then quenched with water 20 mL and 50 ml EtOAc. The combined organic phase was washed with Brine 20 mL, dried over Na₂SO₄ and concentrated under reduced pressure to obtain 2,3-dimethyl-5-[rac-(2R,5S)-5-methyl-2-piperidyl]pyridine (0.46 g, 2.25 mmol, 28.47% yield), which was used in next step without farther purification.

LCMS(ESI): [M+H]⁺ m/z: calcd 205.2; found 205.2; Rt=0.515 min.

### Step 4: The Synthesis of 2,2,2-Trifluoroethyl 2-oxo-2-[rac-(2R,5S)-2-(5,6-dimethyl-3-pyridyl)-5-methyl-1-piperidyl]acetate

2,3-Dimethyl-5-[rac-(2R,5S)-5-methyl-2-piperidyl]pyridine (0.46 g, 2.25 mmol) was dissolved in DCM (19.53 mL) and TEA (341.74 mg, 3.38 mmol, 470.72 μL) was added. Then reaction mixture was cooled and 2,2,2-trifluoroethyl 2-chloro-2-oxo-acetate (514.70 mg, 2.70 mmol) was added dropwise. The reaction was stirred at rt overnight. After that water (50 mL) was added and organic layer was washed with brine (20 mL), dried over Na₂SO₄ and evaporated in vacuum to afford 2,2,2-trifluoroethyl 2-oxo-2-[rac-(2R,5S)-2-(5,6-dimethyl-3-pyridyl)-5-methyl-1-piperidyl]acetate (0.69 g, 1.93 mmol, 85.52% yield).

LCMS(ESI): [M+H]⁺ m/z: calcd 359.2; found 359.2; Rt=0.732 min.

### Step 5: The Synthesis of 2-Oxo-2-[rac-(2R,5S)-2-(5,6-dimethyl-3-pyridyl)-5-methyl-1-piperidyl]acetamide

2,2,2-Trifluoroethyl 2-oxo-2-[rac-(2R,5S)-2-(5,6-dimethyl-3-pyridyl)-5-methyl-1-piperidyl]acetate (0.69 g, 1.93 mmol) was dissolved in NH₃/MeOH (1.93 mmol, 20 mL) and stirred overnight at rt. Then it was evaporated in vacuum and subjected to HPLC (2-10 min 50-70% MeOH+NH₃; flow: 30 mL/min) to afford 2-oxo-2-[rac-(2R,5S)-2-(5,6-dimethyl-3-pyridyl)-5-methyl-1-piperidyl]acetamide (0.5 g, 1.82 mmol, 94.31% yield).

LCMS(ESI): [M+H]⁺ m/z: calcd 276.2; found 276.2; Rt=0.695 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-2-(5,6-dimethyl-3-pyridyl)-5-methyl-1-piperidyl]acetamide

A mixture of 7-bromo-1-(2-trimethylsilylethoxymethyl)pyrazolo[4,3-c]pyridin-4-amine (274.29 mg, 799.00 μmol), 2-oxo-2-[rac-(2R,5S)-2-(5,6-dimethyl-3-pyridyl)-5-methyl-1-piperidyl]acetamide (0.2 g, 726.36 μmol), caesium carbonate (354.99 mg, 1.09 mmol), rac-(1R,2R)—N1,N2-dimethylcyclohexane-1,2-diamine (82.66 mg, 581.09 μmol) and CuI (83.00 mg, 435.82 μmol, 14.77 μL) with a few mg of Cu (2.31 mg, 36.32 μmol) in dioxane (3.99 mL) was stirred in a sealed vial under argon at 105° C. for 48 hr. The resulting mixture was cooled down and submitted to reverse phase HPLC (column: SunFire C18 100×19 mm, 5 um; mobile phase: 30-70% 2-10 min MeOH, flow rate: 30 mL/min) to afford N-[4-amino-1-(2-trimethylsilylethoxymethyl)pyrazolo[4,3-c]pyridin-7-yl]-2-oxo-2-[rac-(2R,5S)-2-(5,6-dimethyl-3-pyridyl)-5-methyl-1-piperidyl]acetamide (73.9 mg, 137.43 μmol, 18.92% yield).

LCMS(ESI): [M+H]⁺ m/z: calcd 538.2; found 538.2; Rt=0.945 min.

### Step 7: The Synthesis of N-(4-Amino-1H-pyrazolo[4,3-c]pyridin-7-yl)-2-oxo-2-[rac-(2R,5S)-2-(5,6-dimethyl-3-pyridyl)-5-methyl-1-piperidyl]acetamide (Compound 241)

N-[4-Amino-1-(2-trimethylsilylethoxymethyl)pyrazolo[4,3-c]pyridin-7-yl]-2-oxo-2-[rac-(2R,5S)-2-(5,6-dimethyl-3-pyridyl)-5-methyl-1-piperidyl]acetamide (73.9 mg, 137.43 μmol) was dissolved in MeOH (2 mL) and diox/HCl (137.43 μmol, 2 mL) was added. Then mixture was stirred at rt 16 hr. The solution was evaporated and submitted to reverse phase HPLC (column: SunFire C18 100×19 mm, 5 um; mobile phase: 10-30% 2-10 min MeCN+FA, flow rate: 30 mK/min). After that crude 33.9 mg was subjected to chiral separation (Column: Chiralpak AD-H (250*20, 5 mkm); Hexane-IPA-MeOH, 50-25-25; Flow rate: 12 mL/min; RT=24.24 min) to afford N-(4-amino-1H-pyrazolo[4,3-c]pyridin-7-yl)-2-oxo-2-[rac-(2R,5S)-2-(5,6-dimethyl-3-pyridyl)-5-methyl-1-piperidyl]acetamide (21.22 mg, 46.79 μmol, 34.05% yield, HCOOH)

Rel Time for Compound 241 in analytical conditions (column: AD-H, Hexane (0.1% EDA)-IPA-MeOH, 50-25-25, 0.6 ml/min as mobile phase) 20.15 min.

¹H NMR (600 MHz, dmso) δ 0.88-1.05 (m, 3H), 1.29-1.42 (m, 1H), 1.56-1.83 (m, 1H), 1.84-1.98 (m, 1H), 1.99-2.21 (m, 2H), 2.21-2.29 (m, 3H), 2.36-2.43 (m, 3H), 2.73-3.25 (m, 1H), 3.73-4.11 (m, 1H), 4.96-5.65 (m, 1H), 6.52-6.89 (m, 2H), 7.42-7.55 (m, 1H), 7.60-7.86 (m, 1H), 8.12-8.27 (m, 2H), 9.42-10.60 (m, 1H), 12.37-13.38 (m, 1H).

LCMS(ESI): [M+H]⁺ m/z: calcd 408.2; found 408.2; Rt=1.449 min.

### Example 215. N-(4-Amino-1H-pyrazolo[4,3-c]pyridin-7-yl)-2-oxo-2-[(2R,5S)-2-(5-fluoro-3-pyridyl)-5-methyl-1-piperidyl]acetamide (Compound 243)

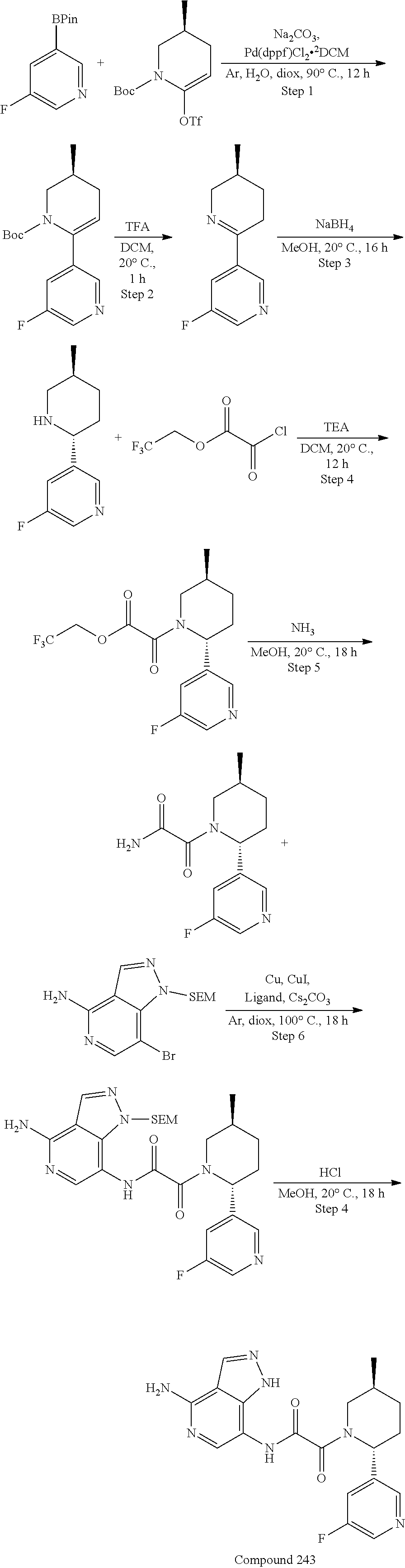

### Step 1: The Synthesis of tert-Butyl rac-(3S)-6-(5-fluoro-3-pyridyl)-3-methyl-3,4-dihydro-2H-pyridine-1-carboxylate

3-Fluoro-5-(4,4,5,5-tetramethyl-1,3,2-dioxaborolan-2-yl)pyridine (2 g, 8.97 mmol), tert-butyl rac-(3S)-3-methyl-6-(trifluoromethylsulfonyloxy)-3,4-dihydro-2H-pyridine-1-carboxylate (3.41 g, 9.86 mmol), Sodium carbonate (1.90 g, 17.93 mmol, 750.68 μL) and Pd(dppf)Cl₂·DCM (366.12 mg, 448.33 μmol) were mixed in dioxane (20 mL)+water (5 mL) under argon, and then stirred overnight at 90° C. for 12 hr in vial. The solid was filtered off, the filtrate was diluted with EA, the organic layer was separated and the aqueous layer was extracted with additional EA, the combined organic layer was washed with brine, dried over Na₂SO₄, filtered and concentrated in vacuo. The obtained product was used in the next step without further purification. The reaction was successful. tert-Butyl rac-(3S)-6-(5-fluoro-3-pyridyl)-3-methyl-3,4-dihydro-2H-pyridine-1-carboxylate (4.16 g, crude) was obtained as a red gum.

LCMS(ESI): [M+H]⁺ m/z: calcd 293.2; found 293.2; Rt=1.195 min.

### Step 2: The Synthesis of 3-Fluoro-5-[rac-(3S)-3-methyl-2,3,4,5-tetrahydropyridin-6-yl]pyridine

tert-Butyl rac-(3S)-6-(5-fluoro-3-pyridyl)-3-methyl-3,4-dihydro-2H-pyridine-1-carboxylate (4.16 g, 14.23 mmol) was dissolved in a mixture of TFA (12 mL) and DCM (12 mL). The resulting clear solution was stirred for 1 hr at 20° C. The reaction mixture was concentrated on vacuo. The obtained residue was dissolved in water and extracted with MTBE (3 times), after that the aqueous layer was basified with NaOH and extracted with DCM (3 times), combined DCM layers was dried over Na₂SO₄, filtered and evaporated. The reaction is successful. 3-Fluoro-5-[rac-(3S)-3-methyl-2,3,4,5-tetrahydropyridin-6-yl]pyridine (1.6 g, 8.32 mmol, 58.49% yield) was obtained as a red gum.

### Step 3: The Synthesis of 3-Fluoro-5-[rac-(2R,5S)-5-methyl-2-piperidyl]pyridine

To a stirring suspension of 3-fluoro-5-[rac-(3S)-3-methyl-2,3,4,5-tetrahydropyridin-6-yl]pyridine (1.6 g, 8.32 mmol) in MeOH (30 mL) was added Sodium Borohydride (629.78 mg, 16.65 mmol, 586.39 μL) in portions. Resulting mixture was stirred at 20° C. for 12 hr. The solvent was removed under reduced pressure and residue was partitioned between water and DCM. The organic layer was separated, dried over Na₂SO₄ and concentrated in vacuo. The obtained product was used in the next step without further purification. The reaction is successful. 3-Fluoro-5-[rac-(2R,5S)-5-methyl-2-piperidyl]pyridine (1.2 g, 6.18 mmol, 74.22% yield) was obtained as a red oil.

LCMS(ESI): [M+H]⁺ m/z: calcd 195.2; found 195.2; Rt=0.654 min.

### Step 4: The Synthesis of 2,2,2-Trifluoroethyl 2-oxo-2-[rac-(2R,5S)-2-(5-fluoro-3-pyridyl)-5-methyl-1-piperidyl]acetate

2,2,2-Trifluoroethyl 2-chloro-2-oxo-acetate (1.29 g, 6.80 mmol) was added dropwise to a solution of 3-fluoro-5-[rac-(2R,5S)-5-methyl-2-piperidyl]pyridine (1.2 g, 6.18 mmol) and TEA (781.40 mg, 7.72 mmol, 1.08 mL) in DCM (35 mL) at 0° C. After addition was complete, cooling bath was removed and resulting mixture was allowed to warm up to 20° C. and stirred for 12 hr. The reaction mixture was washed with water, dried over Na₂SO₄ and concentrated under reduced pressure. The obtained product was used in the next step without further purification. The reaction is successful. 2,2,2-Trifluoroethyl 2-oxo-2-[rac-(2R,5S)-2-(5-fluoro-3-pyridyl)-5-methyl-1-piperidyl]acetate (2 g, 5.74 mmol, 92.95% yield) was obtained as a brown gum.

LCMS(ESI): [M+H]⁺ m/z: calcd 349.2; found 349.2; Rt=1.264 min.

### Step 5: The Synthesis of 2-Oxo-2-[rac-(2R,5S)-2-(5-fluoro-3-pyridyl)-5-methyl-1-piperidyl]acetamide

2,2,2-Trifluoroethyl 2-oxo-2-[rac-(2R,5S)-2-(5-fluoro-3-pyridyl)-5-methyl-1-piperidyl]acetate (2 g, 5.74 mmol) was dissolved in MeOH/NH₃ (35 mL) as stirred overnight at 20° C. The reaction mixture was evaporated to dryness. The obtained product was used in the next step without further purification. The reaction is successful. 2-Oxo-2-[rac-(2R,5S)-2-(5-fluoro-3-pyridyl)-5-methyl-1-piperidyl]acetamide (2 g, crude) was obtained as an orange gum.

LCMS(ESI): [M+H]⁺ m/z: calcd 266.2; found 266.2; Rt=0.947 min.

### Step 6: The Synthesis of N-[4-amino-1-(2-trimethylsilylethoxymethyl)pyrazolo[4,3-c]pyridin-7-yl]-2-oxo-2-[(2R,5S)-2-(5-fluoro-3-pyridyl)-5-methyl-1-piperidyl]acetamide

7-Bromo-1-(2-trimethylsilylethoxymethyl)pyrazolo[4,3-c]pyridin-4-amine (380 mg, 1.11 mmol), 2-oxo-2-[(2R,5S)-2-(5-fluoro-3-pyridyl)-5-methyl-1-piperidyl]acetamide (293.65 mg, 1.11 mmol), Cu (7.46 mg, 117.33 μmol), CuI (63.24 mg, 332.08 μmol, 11.25 μL), Caesium carbonate (540.99 mg, 1.66 mmol, 236.24 μL) and (1R,2R)—N1,N2-dimethylcyclohexane-1,2-diamine (47.23 mg, 332.08 μmol) were mixed in dioxane (5 mL), purged with Ar for 5 minutes and then heated in the sealed tube at 100° C. for 18 hr. Final mixture was filtered and dioxane was evaporated in vacuo. The residue was purified by RP-HPLC (column: XBridge BEH C18 5 um 130 A; 40-40-70% 0-1-6 min H₂O/ACN/0.1% NH₄OH, flow: 30 mL/min, flow: 30 mL/min) to afford N-[4-amino-1-(2-trimethylsilylethoxymethyl)pyrazolo[4,3-c]pyridin-7-yl]-2-oxo-2-[(2R,5S)-2-(5-fluoro-3-pyridyl)-5-methyl-1-piperidyl]acetamide (204 mg, 386.61 μmol, 34.93% yield) as light-brown solid (mixture of cis/trans), which was used directly in the next step.

LCMS(ESI): [M+H]⁺ m/z: calcd 528.4; found 528.4; Rt=1.270 min.

### Step 7: The Synthesis of N-(4-Amino-1H-pyrazolo[4,3-c]pyridin-7-yl)-2-oxo-2-[(2R,5S)-2-(5-fluoro-3-pyridyl)-5-methyl-1-piperidyl]acetamide (Compound 243)

To a solution of N-[4-amino-1-(2-trimethylsilylethoxymethyl)pyrazolo[4,3-c]pyridin-7-yl]-2-oxo-2-[(2R,5S)-2-(5-fluoro-3-pyridyl)-5-methyl-1-piperidyl]acetamide (204 mg, 386.61 μmol) in methanol (2 mL) was added Hydrogen chloride solution 4.0M in dioxane (2.11 g, 5.80 mmol, 2.64 mL, 10% purity) at 21° C. The resulting mixture was left to stir for 18 hr. The resulting mixture was evaporated to dryness. The residue was purified by RP-HPLC (column: YMC Triart C18 100×20 mm, 5 um; 10-50% 0-5 min H₂O/MeOH/0.1% NH₄OH, flow: 30 mL/min) to give Compound 243 N-(4-amino-1H-pyrazolo[4,3-c]pyridin-7-yl)-2-oxo-2-[(2R,5S)-2-(5-fluoro-3-pyridyl)-5-methyl-1-piperidyl]acetamide (55 mg, 138.40 μmol, 35.80% yield) and 25 mg crude fraction with 24% cis impurity.

¹H NMR (600 MHz, dmso) δ 0.82-1.09 (m, 3H), 1.29-1.46 (m, 1H), 1.55-1.97 (m, 2H), 2.01-2.31 (m, 2H), 2.69-3.17 (m, 1H), 3.72-4.16 (m, 1H), 5.10-5.73 (m, 1H), 6.63-6.87 (m, 2H), 7.47-8.22 (m, 3H), 8.30-8.57 (m, 2H), 9.63-10.61 (m, 1H), 12.61-13.35 (m, 1H).

LCMS(ESI): [M+H]⁺ m/z: calcd 398.2; found 398.2; Rt=1.708 min.

### Example 216. N-(4-amino-1H-pyrazolo[4,3-c]pyridin-7-yl)-2-(rac-(2S,5R)-2-isopropyl-5-methylpiperidin-1-yl)-2-oxoacetamide (Compound 244)

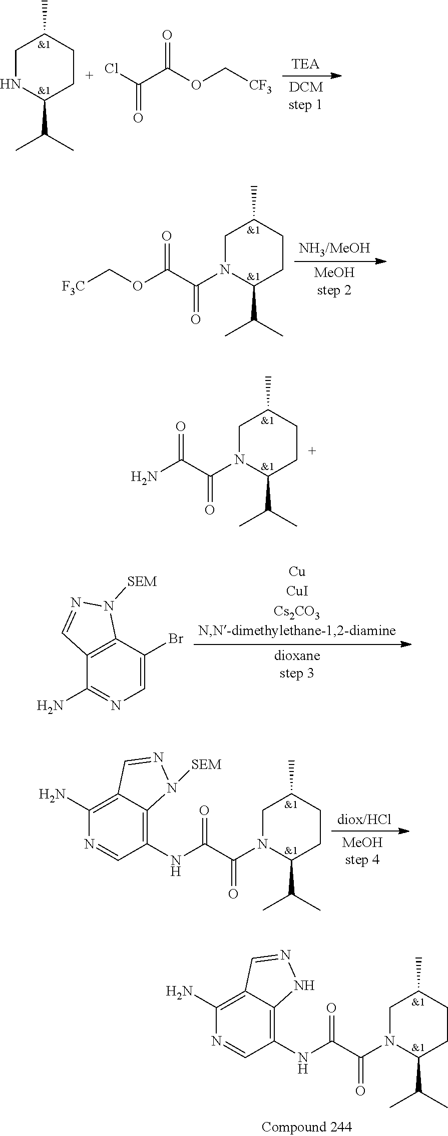

### Step 1: Synthesis of rac-2,2,2-trifluoroethyl 2-((2R,5S)-2-isopropyl-5-methylpiperidin-1-yl)-2-oxoacetate

rac-(2S,5R)-2-isoPropyl-5-methyl-piperidine (1.2 g, 6.75 mmol, HCl) was dissolved in DCM (16.71 mL) and TEA (2.39 g, 23.63 mmol, 3.29 mL) was added. Then reaction mixture was cooled and 2,2,2-trifluoroethyl 2-chloro-2-oxo-acetate (1.29 g, 6.75 mmol) was added dropwise. The reaction was stirred at rt overnight. After that water (50 ml) was added and organic layer was washed with brine (20 ml), dried over Na₂SO₄ and evaporated in vacuum to afford 2,2,2-trifluoroethyl 2-oxo-2-[rac-(2S,5R)-2-isopropyl-5-methyl-1-piperidyl]acetate (1 g, 3.39 mmol, 50.15% yield).

LCMS(ESI): [M+1]⁺ m/z: calcd 295.2; found 296.2; Rt=1.207 min.

### Step 2: Synthesis of rac-2-((2R,5S)-2-isopropyl-5-methylpiperidin-1-yl)-2-oxoacetamide

2,2,2-Trifluoroethyl 2-oxo-2-[rac-(2S,5R)-2-isopropyl-5-methyl-1-piperidyl]acetate (1 g, 3.39 mmol) was dissolved in MeOH/NH₃ (15 mL) and stirred overnight at rt. Then it was evaporated in vacuum to afford 2-oxo-2-[rac-(2S,5R)-2-isopropyl-5-methyl-1-piperidyl]acetamide (0.6 g, 2.83 mmol, 83.46% yield).

LCMS(ESI): [M+1]⁺ m/z: calcd 212.2; found 213.2; Rt=1.086 min.

### Step 3: Synthesis of rac-N-(4-amino-1-((2-(trimethylsilyl)ethoxy)methyl)-1H-pyrazolo[4,3-c]pyridin-7-yl)-2-((2R,5S)-2-isopropyl-5-methylpiperidin-1-yl)-2-oxoacetamide

A mixture of 7-bromo-1-(2-trimethylsilylethoxymethyl)pyrazolo[4,3-c]pyridin-4-amine (533.65 mg, 1.55 mmol), 2-oxo-2-[rac-(2S,5R)-2-isopropyl-5-methyl-1-piperidyl]acetamide (0.3 g, 1.41 mmol), cesium carbonate (690.66 mg, 2.12 mmol), (1R,2R)—N1,N2-dimethylcyclohexane-1,2-diamine (160.81 mg, 1.13 mmol) and CuI (161.48 mg, 847.91 μmol, 28.73 μL) with a few mg of Cu (4.49 mg, 70.66 μmol) in dioxane (4.97 mL) was stirred in a sealed vial under argon at 105° C. for 48 hr. The resulting mixture was cooled down and submitted to reverse phase HPLC (column: SunFire C18 100×19 mm, 5 um; mobile phase: 30-70% 2-10 min MeCN, flow rate: 30 ml/min) to afford N-[4-amino-1-(2-trimethylsilylethoxymethyl)pyrazolo[4,3-c]pyridin-7-yl]-2-oxo-2-[rac-(2S,5R)-2-isopropyl-5-methyl-1-piperidyl]acetamide (75.8 mg, 159.69 μmol, 11.30% yield).

LCMS(ESI): [M+1]⁺ m/z: calcd 474.2; found 475.2; Rt=1.187 min.

### Step 4: Synthesis of N-(4-amino-1H-pyrazolo[4,3-c]pyridin-7-yl)-2-(rac-(2S,5R)-2-isopropyl-5-methylpiperidin-1-yl)-2-oxoacetamide (Compound 244)

N-[4-Amino-1-(2-trimethylsilylethoxymethyl)pyrazolo[4,3-c]pyridin-7-yl]-2-oxo-2-[rac-(2S,5R)-2-isopropyl-5-methyl-1-piperidyl]acetamide (75.8 mg, 159.69 μmol) was dissolved in MeOH (2 mL) and diox/HCl (159.69 μmol, 2 mL) was added. Then mixture was stirred at rt 16 hr. The solution was evaporated and submitted to reverse phase HPLC (column: SunFire C18 100×19 mm, 5 um; mobile phase: 0-50% 2-10 min MeCN+FA, flow rate: 30 ml/min) to afford N-(4-amino-1H-pyrazolo[4,3-c]pyridin-7-yl)-2-oxo-2-[rac-(2S,5R)-2-isopropyl-5-methyl-1-piperidyl]acetamide (26.4 mg, 67.62 μmol, 42.34% yield, HCOOH).

### Compound 244:

¹H NMR (600 MHz, DMSO-d₆) δ (ppm) 0.23-0.82 (m, 3H), 0.82-1.02 (m, 6H), 1.17-1.47 (m, 1H), 1.51-1.91 (m, 3H), 1.94-2.26 (m, 1H), 2.89-3.09 (m, 1H), 3.30-3.31 (m, 1H), 3.59-3.67 (m, 1H), 3.90-4.13 (m, 1H), 6.61-7.51 (m, 2H), 7.72-8.18 (m, 2H), 9.27-10.49 (m, 1H), 12.40-13.45 (m, 1H).

LCMS(ESI): [M+1]⁺ m/z: calcd 344.2; found 345.2; Rt=2.566 min.

### Example 217. N-(4-Amino-2H-pyrazolo[4,3-c]pyridin-7-yl)-2-oxo-2-[rac-(2R,6S)-2-phenyl-6-(trifluoromethyl)-1-piperidyl]acetamide (Compound 245)

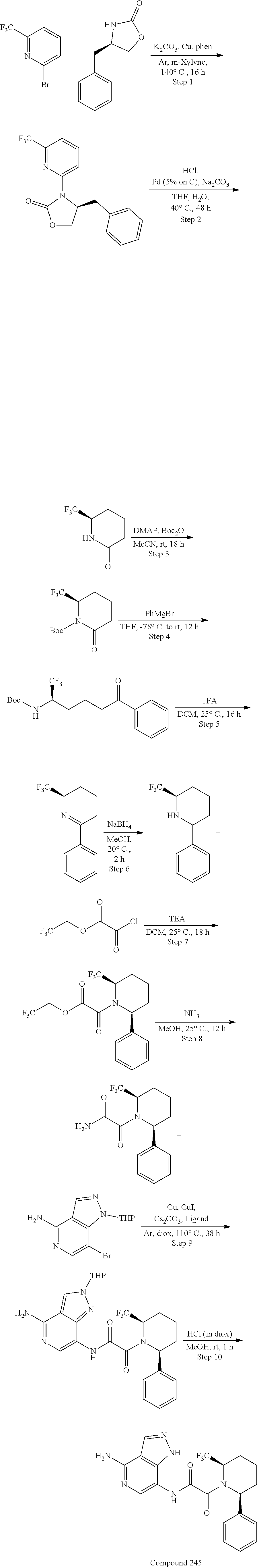

### Step 1: The Synthesis of rac-(4R)-4-Benzyl-3-[6-(trifluoromethyl)-2-pyridyl]oxazolidin-2-one

An oven-dried Schlenk tube was charged with 2-bromo-6-(trifluoromethyl)pyridine (10 g, 44.25 mmol), rac-(4R)-4-benzyloxazolidin-2-one (7.84 g, 44.25 mmol), Potassium carbonate (7.95 g, 57.52 mmol, 3.47 mL), CuI (842.72 mg, 4.42 mmol, 149.95 μL) and 1,10-phenanthroline (1.20 g, 6.64 mmol) and was put under an argon atmosphere. Dry toluene (0.5 M) was added to the solids and the mixture was stirred at 140° C. for 16 h. After cooling to room temperature, the reaction mixture was filtered through a silica pad and concentrated in vacuo. The residue was purified by column chromatography on silica gel (MTBE/Hexane from 0% to 100%) to obtain rac-(4R)-4-benzyl-3-[6-(trifluoromethyl)-2-pyridyl]oxazolidin-2-one (9.8 g, 30.41 mmol, 68.72% yield).

### Step 2: The Synthesis of rac-(6S)-6-(Trifluoromethyl)piperidin-2-one

To an autoclave rac-(4R)-4-benzyl-3-[6-(trifluoromethyl)-2-pyridyl]oxazolidin-2-one (9.8 g, 30.41 mmol) and Palladium, 5% on activated carbon paste, 5R437 (6.47 g, 3.04 mmol, 5% purity) were charged. THF (125 mL) and Water (125 mL) were added thereto followed by addition of Hydrochloric acid ACS grade 36-38% (7.39 g, 72.98 mmol, 6.37 mL, 36% purity). The autoclave was pressurized and depressurized three times with hydrogen gas before the final hydrogen pressure was set to 50 atm. The reaction mixture was stirred at 50 atm at 40° C. for 48 hr. The autoclave was cooled to room temperature and carefully depressurized. The reaction mixture was carefully poured onto Sodium carbonate (12.89 g, 121.63 mmol, 5.09 mL) and the resulting mixture was filtered. The filter cake was rinsed with THF (150 mL) and the filtrate was concentrated in vacuo. The residue was re-dissolved in water (75 mL) and extracted with DCM (2*100 mL). Combined organic layers were dried over Na₂SO₄, filtered, and concentrated in vacuo. The residue was distilled under reduced pressure (1 torr) to obtain rac-(6S)-6-(trifluoromethyl)piperidin-2-one (845 mg, 5.06 mmol, 16.63% yield).

¹H NMR (500 MHZ, DMSO-d₆) δ (ppm) 1.74 (m, 3H), 1.93 (m, 1H), 2.16 (m, 2H), 4.05 (m, 1H), 8.02 (s, 1H).

### Step 3: The Synthesis of tert-Butyl rac-(6S)-2-oxo-6-(trifluoromethyl)piperidine-1-carboxylate

rac-(6S)-6-(Trifluoromethyl)piperidin-2-one (0.845 g, 5.06 mmol) was dissolved in Acetonitrile (13 mL) and DMAP (61.77 mg, 505.60 μmol) was added thereto. Di-tert-butyl dicarbonate (1.21 g, 5.56 mmol, 1.28 mL) was added dropwise and the resulting mixture was stirred overnight at rt. The reaction mixture was concentrated in vacuo and the residue was dissolved in DCM (50 mL). The resulting solution was washed with 10% aq.citric acid solution (35 mL), dried over Na₂SO₄, filtered and concentrated in vacuo to obtain tert-butyl rac-(6S)-2-oxo-6-(trifluoromethyl)piperidine-1-carboxylate (1.06 g, crude).

LCMS(ESI): [M-Boc]⁺ m/z: calcd 168.2; found 168.2; Rt=1.310 min.

### Step 4: The Synthesis of tert-Butyl N-[rac-(1S)-5-oxo-5-phenyl-1-(trifluoromethyl)pentyl]carbamate

To a dry 2 necked flask was added THF (15 mL) and tert-butyl rac-(6S)-2-oxo-6-(trifluoromethyl)piperidine-1-carboxylate (1.06 g, 3.97 mmol) with stirring and the solution was cooled to −78° C. Phenyl magnesium bromide (6.23 g, 5.16 mmol, 6.36 mL, 15% purity) was added to the t-Boc-lactam over 1 hour, maintaining the internal temperature below −70° C. The solution was warmed to rt and was stirred for 12 hrs. Sat. aq. NH₄Cl solution was added (10 mL). The aqueous layer was extracted (3×10 mL) with DCM and the organic layers combined, dried over Na₂SO₄, filtered and concentrated in vacuo. tert-Butyl N-[rac-(1S)-5-oxo-5-phenyl-1-(trifluoromethyl)pentyl]carbamate (0.96 g, crude) was obtained as a light-yellow oil and was used in the next step without further purification.

LCMS(ESI): [M-Boc]⁺ m/z: calcd 246.0; found 246.0; Rt=1.311 min.

### Step 5: The Synthesis of rac-(2S)-6-Phenyl-2-(trifluoromethyl)-2,3,4,5-tetrahydropyridine

Trifluoroacetic acid (4.75 g, 41.70 mmol, 3.21 mL) was added to the solution of tert-butyl N-[rac-(1S)-5-oxo-5-phenyl-1-(trifluoromethyl)pentyl]carbamate (0.96 g, 2.78 mmol) in DCM (3 mL). Resulting mixture was stirred at 25° C. for 16 hr. Then, volatiles were removed under reduced pressure and residue was partitioned between 20% aq. K₂CO₃ solution (30 mL) and ethyl acetate (50 mL). Organic layer was separated, dried over Na₂SO₄ and concentrated in vacuo, affording rac-(2S)-6-phenyl-2-(trifluoromethyl)-2,3,4,5-tetrahydropyridine (0.43 g, crude).

LCMS(ESI): [M+H]⁺ m/z: calcd 228.0; found 228.0; Rt=1.296 min.

### Step 6: The Synthesis of rac-(6S)-2-Phenyl-6-(trifluoromethyl)piperidine

rac-(2S)-6-Phenyl-2-(trifluoromethyl)-2,3,4,5-tetrahydropyridine (430 mg, 1.89 mmol) was dissolved in Methanol (10.06 mL) and Sodium Borohydride (107.39 mg, 2.84 mmol, 99.99 μL) was added portionwise. After the addition was completed, the reaction mixture was stirred for 3 hr. Water (5 mL) was added to the reaction mixture and the resulting mixture was concentrated in vacuo. Water (10 mL) was added to the residue and the resulting mixture was extracted with DCM (2*10 mL). Combined organic layers were concentrated in vacuo. Resulting crude product was diluted with water (20 mL) and acidified to pH 3 with NaHSO₄, resulting mixture was washed with DCM (2×20 mL). Aqueous solution was basified with potassium carbonate to pH 9 and extracted with DCM (2×20 mL). Organic phase was separated, dried over Na₂SO₄, filtered and evaporated to obtain rac-(6S)-2-phenyl-6-(trifluoromethyl)piperidine (120 mg, crude).

LCMS(ESI): [M+H]⁺ m/z: calcd 230.0; found 230.0; Rt=0.836 min.

### Step 7: The Synthesis of 2,2,2-Trifluoroethyl 2-oxo-2-[rac-(2R,6S)-2-phenyl-6-(trifluoromethyl)-1-piperidyl]acetate

rac-(6S)-2-Phenyl-6-(trifluoromethyl)piperidine (120 mg, 523.47 μmol) and Triethylamine (63.56 mg, 628.16 μmol, 87.55 μL) were dissolved in DCM (1.95 mL) and the resulting solution was cooled to 25° C. in an ice/methanol bath. A solution of 2,2,2-trifluoroethyl 2-chloro-2-oxo-acetate (109.70 mg, 575.81 μmol, 74.62 μL) in DCM (975.13 μL) was added dropwise at 25° C. and the resulting mixture was allowed to warm to room temperature and stirred overnight. Water (5 mL) was added to the residue and an organic layer was separated. The aqueous layer was extracted with DCM (15 mL) and combined organic layers were dried over Na₂SO₄, filtered, and concentrated in vacuo to obtain 2,2,2-trifluoroethyl 2-oxo-2-[rac-(2R,6S)-2-phenyl-6-(trifluoromethyl)-1-piperidyl]acetate (170 mg, crude).

LCMS(ESI): [M+H]⁺ m/z: calcd 384.0; found 384.0; Rt=1.451 min.

### Step 8: The Synthesis of 2-Oxo-2-[rac-(2R,6S)-2-phenyl-6-(trifluoromethyl)-1-piperidyl]acetamide

A solution of 2,2,2-trifluoroethyl 2-oxo-2-[rac-(2R,6S)-2-phenyl-6-(trifluoromethyl)-1-piperidyl]acetate (170 mg, 443.53 μmol) in Methanol/NH₃ (7N) (2 mL) was stirred at 25° C. for 12 hr. The solvent was evaporated to obtain 2-oxo-2-[rac-(2R,6S)-2-phenyl-6-(trifluoromethyl)-1-piperidyl]acetamide (121 mg, crude).

LCMS(ESI): [M+H]⁺ m/z: calcd 301.0; found 301.0; Rt=1.246 min.

### Step 9: The Synthesis of N-(4-Amino-2-tetrahydropyran-2-yl-pyrazolo[4,3-c]pyridin-7-yl)-2-oxo-2-[rac-(2R,6S)-2-phenyl-6-(trifluoromethyl)-1-piperidyl]acetamide

To an 8 mL vial 2-oxo-2-[rac-(2R,6S)-2-phenyl-6-(trifluoromethyl)-1-piperidyl]acetamide (130 mg, 432.94 μmol), 7-bromo-2-tetrahydropyran-2-yl-pyrazolo[4,3-c]pyridin-4-amine (141.51 mg, 476.23 μmol), Copper (1.38 mg, 21.65 μmol), Copper (I) iodide (41.23 mg, 216.47 μmol, 7.34 μL), rac-(1R,2R)—N1,N2-dimethylcyclohexane-1,2-diamine (46.19 mg, 324.70 μmol), Cesium carbonate (282.12 mg, 865.87 μmol) and Dioxane (3 mL) were charged and the resulting mixture was spurred with argon for 5 min. The vial was sealed and heated at 110° C. for 38 hr. The reaction mixture was cooled, diluted with MeOH (5 mL), and filtered. The filtrate was concentrated in vacuo. The residue was purified by HPLC (0-2-10 min 43-50-70% H₂O/MeOH/0.1NH₄OH, flow 30 mL/min (loading pump 4 mL MeOH; column: Chromatorex SMB100-5T C18 100*19 mm, 5 microM) to obtain N-(4-amino-2-tetrahydropyran-2-yl-pyrazolo[4,3-c]pyridin-7-yl)-2-oxo-2-[rac-(2R,6S)-2-phenyl-6-(trifluoromethyl)-1-piperidyl]acetamide (32 mg, 61.95 μmol, 14.31% yield).

LCMS(ESI): [M+H]⁺ m/z: calcd 517.2; found 517.2; Rt=1.194 min.

### Step 10: The Synthesis of N-(4-Amino-2H-pyrazolo[4,3-c]pyridin-7-yl)-2-oxo-2-[rac-(2R,6S)-2-phenyl-6-(trifluoromethyl)-1-piperidyl]acetamide (Compound 245)

N-(4-Amino-2-tetrahydropyran-2-yl-pyrazolo[4,3-c]pyridin-7-yl)-2-oxo-2-[rac-(2R,6S)-2-phenyl-6-(trifluoromethyl)-1-piperidyl]acetamide (32 mg, 61.95 μmol) was dissolved in Methanol (1 mL) and Dioxane/HCl (1 mL) was added. The resulting solution was stirred for 1 hr and the reaction mixture was concentrated in vacuo. The residue was purified by HPLC (0-2-10 min 0-85% H₂O/ACN/0.1FA; flow 30 mL/min (loading pump 4 mL ACN), COLUMN: Chromatorex C18 SMB100-5T 100*19 mm, 5 microM) to obtain N-(4-amino-2H-pyrazolo[4,3-c]pyridin-7-yl)-2-oxo-2-[rac-(2R,6S)-2-phenyl-6-(trifluoromethyl)-1-piperidyl]acetamide (9.1 mg, 19.02 μmol, 30.70% yield, HCOOH) and N-(4-amino-2H-pyrazolo[4,3-c]pyridin-7-yl)-2-oxo-2-[rac-(2R,6S)-2-phenyl-6-(trifluoromethyl)-1-piperidyl]acetamide (4.7 mg, 9.82 μmol, 15.86% yield, HCOOH).

¹H NMR (600 MHz, DMSO-d₆) δ (ppm) 1.57-1.71 (m, 1H), 1.73-1.84 (m, 1H), 1.87-2.11 (m, 3H), 2.83-3.17 (m, 1H), 5.11-5.48 (m, 1H), 5.48-5.84 (m, 1H), 6.62-6.90 (m, 2H), 7.19-7.24 (m, 1H), 7.25-7.36 (m, 3H), 7.38-7.47 (m, 1H), 7.49-7.74 (m, 1H), 8.14-8.26 (m, 1H), 10.44-10.83 (m, 1H), 12.46-12.95 (m, 1H).

LCMS(ESI): [M+H]⁺ m/z: calcd 433.0; found 433.0; Rt=1.051 min.

### Example 218. N-(4-Amino-1H-pyrazolo[4,3-c]pyridin-7-yl)-2-oxo-2-[rac-(2R,5S)-2-(6-methoxy-5-methyl-3-pyridyl)-5-methyl-1-piperidyl]acetamide (Compound 246)

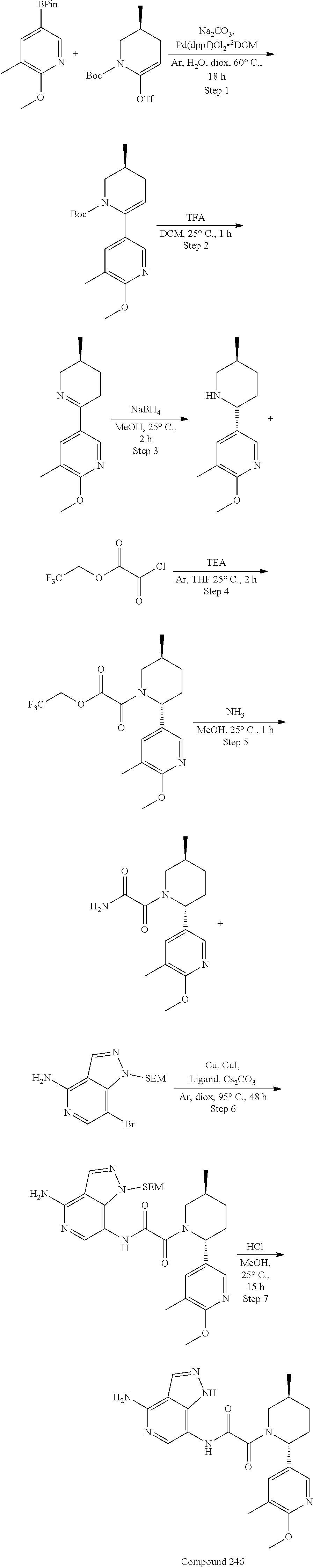

### Step 1: The Synthesis of tert-Butyl rac-(3S)-6-(6-methoxy-5-methyl-3-pyridyl)-3-methyl-3,4-dihydro-2H-pyridine-1-carboxylate

Sodium carbonate (2.32 g, 21.90 mmol, 916.56 μL) was added to a solution of tert-butyl rac-(3S)-3-methyl-6-(trifluoromethylsulfonyloxy)-3,4-dihydro-2H-pyridine-1-carboxylate (3.78 g, 10.95 mmol) and 2-methoxy-3-methyl-5-(4,4,5,5-tetramethyl-1,3,2-dioxaborolan-2-yl)pyridine (3 g, 12.04 mmol) in Dioxane (25 mL) and Water (8 mL). Reaction flask was evacuated and refilled with argon 3 times. Then, Pd(dppf)Cl₂*DCM (894.05 mg, 1.09 mmol) was added under stream of argon. Resulting mixture was stirred at 95° C. for 14 hr under inert atmosphere. Reaction solution was decantated and concentrated under reduce pressure to give crude product which was diluted with MTBE (200 mL). The resulting cloudy solution was decanted from oily residue. MTBE was evaporated in vacuo to give tert-butyl rac-(3S)-6-(6-methoxy-5-methyl-3-pyridyl)-3-methyl-3,4-dihydro-2H-pyridine-1-carboxylate (3 g, 9.42 mmol, 86.06% yield).

LCMS(ESI): [M+H]⁺ m/z: calcd 319.2; found 319.4; Rt=1.396 min.

### Step 2: The Synthesis of 2-Methoxy-3-methyl-5-[rac-(3S)-3-methyl-2,3,4,5-tetrahydropyridin-6-yl]pyridine

tert-Butyl rac-(3S)-6-(6-methoxy-5-methyl-3-pyridyl)-3-methyl-3,4-dihydro-2H-pyridine-1-carboxylate (3 g, 9.42 mmol) was diluted with Trifluoroacetic acid (44.40 g, 389.40 mmol, 30 mL). The resulting mixture was stirred at 25° C. for 2 hr. TFA was evaporated in vacuo. The residue was diluted with water (150 mL). The resulting cloudy solution was decanted from oily residue, then basified with NaHCO₃. Product was extracted with DCM (3*50 mL). Combined organic layers were dried over Na₂SO₄. DCM was evaporated in vacuo to give 2-methoxy-3-methyl-5-[rac-(3S)-3-methyl-2,3,4,5-tetrahydropyridin-6-yl]pyridine (1.9 g, 8.70 mmol, 92.38% yield).

LCMS(ESI): [M+H]⁺ m/z: calcd 219.2; found 219.2; Rt=0.691 min.

### Step 3: The Synthesis of 2-Methoxy-3-methyl-5-[rac-(2R,5S)-5-methyl-2-piperidyl]pyridine

To the stirred solution of 2-methoxy-3-methyl-5-[rac-(3S)-3-methyl-2,3,4,5-tetrahydropyridin-6-yl]pyridine (1.9 g, 8.70 mmol) in MeOH (25 mL) Sodium Borohydride (329.29 mg, 8.70 mmol, 306.60 μL) was added portionwise. The reaction mixture was stirred at 25° C. for 2 hr. Methanol was evaporated in vacuo. The residue was diluted with water (15 mL) and extracted with DCM (2*50 mL). Combined organic layer were washed with brine dried over Na₂SO₄. DCM was evaporated to give 2-methoxy-3-methyl-5-[rac-(2R,5S)-5-methyl-2-piperidyl]pyridine (1.3 g, 5.90 mmol, 67.80% yield).

LCMS(ESI): [M+H]⁺ m/z: calcd 221.2; found 221.2; Rt=0.891 min.

### Step 4: The Synthesis of 2,2,2-Trifluoroethyl 2-oxo-2-[rac-(2R,5S)-2-(6-methoxy-5-methyl-3-pyridyl)-5-methyl-1-piperidyl]acetate

To a solution of 2-methoxy-3-methyl-5-[rac-(2R,5S)-5-methyl-2-piperidyl]pyridine (1.3 g, 5.90 mmol) and Triethylamine (1.19 g, 11.80 mmol, 1.64 mL) in THF (29.99 mL), 2,2,2-trifluoroethyl 2-chloro-2-oxo-acetate (1.69 g, 8.85 mmol) was added portionwise at 25° C. The resulting mixture was stirred for 1 hr. LCMS showed full conversion of SM. The reaction mixture was directly used for the next step.

LCMS(ESI): [M+H]⁺ m/z: calcd 375.0; found 375.0; Rt=1.435 min.

### Step 5: The Synthesis of 2-Oxo-2-[rac-(2R,5S)-2-(6-methoxy-5-methyl-3-pyridyl)-5-methyl-1-piperidyl]acetamide

Through a solution of 2,2,2-trifluoroethyl 2-oxo-2-[rac-(2R,5S)-2-(6-methoxy-5-methyl-3-pyridyl)-5-methyl-1-piperidyl]acetate (2 g, 5.34 mmol) in THF (30 mL), Ammonia (909.86 mg, 53.43 mmol) was bubbled during 10 min at 25° C. The formed precipitate was filtered off, washed with THF (30 mL) and the solvent was evaporated in vacuo to give crude product (0.8 g) which was purified by reverse phase HPLC (SYSTEM 25-75% 0-5 min H₂O/MeOH, flow: 30 mL/min (loading pump 4 mL/min MeOH); column: Chromatorex 18 SMB100-5T 100×19 mm 5 um) to give 2-oxo-2-[rac-(2R,5S)-2-(6-methoxy-5-methyl-3-pyridyl)-5-methyl-1-piperidyl]acetamide (0.59 g, 2.03 mmol, 37.91% yield).

LCMS(ESI): [M+H]⁺ m/z: calcd 292.2; found 292.2; Rt=1.125 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-2-(6-methoxy-5-methyl-3-pyridyl)-5-methyl-1-piperidyl]acetamide

A mixture of 2-oxo-2-[rac-(2R,5S)-2-(6-methoxy-5-methyl-3-pyridyl)-5-methyl-1-piperidyl]acetamide (0.25 g, 858.09 μmol), 7-bromo-1-(2-trimethylsilylethoxymethyl)pyrazolo[4,3-c]pyridin-4-amine (294.58 mg, 858.09 μmol), Copper (I) iodide (163.42 mg, 858.09 μmol, 29.08 μL), Cesium carbonate (419.37 mg, 1.29 mmol), (1R,2R)—N1,N2-dimethylcyclohexane-1,2-diamine (183.08 mg, 1.29 mmol) and Cu (5.45 mg, 85.81 μmol) in Dioxane (3 mL) was stirred under argon at 95° C. for 48 hr. The reaction mixture was submitted for reverse phase HPLC (60-60-100% 0-1-6 min H₂O/MeOH/0.1% NH₄OH, flow: 30 mL/min (loading pump 4 mL/min MeOH); column: XBridge BEH C18 5 um 130 A) to give N-[4-amino-1-(2-trimethylsilylethoxymethyl)pyrazolo[4,3-c]pyridin-7-yl]-2-oxo-2-[rac-(2R,5S)-2-(6-methoxy-5-methyl-3-pyridyl)-5-methyl-1-piperidyl]acetamide (149 mg, 269.09 μmol, 31.36% yield).

LCMS(ESI): [M+H]⁺ m/z: calcd 554.2; found 554.2; Rt=1.290 min.

### Step 7: The Synthesis of N-(4-Amino-1H-pyrazolo[4,3-c]pyridin-7-yl)-2-oxo-2-[rac-(2R,5S)-2-(6-methoxy-5-methyl-3-pyridyl)-5-methyl-1-piperidyl]acetamide (Compound 246)

To the stirred solution of N-[4-amino-1-(2-trimethylsilylethoxymethyl)pyrazolo[4,3-c]pyridin-7-yl]-2-oxo-2-[rac-(2R,5S)-2-(6-methoxy-5-methyl-3-pyridyl)-5-methyl-1-piperidyl]acetamide (149 mg, 269.09 μmol) in MeOH (0.5 mL Hydrogen chloride solution 4.0M in dioxane (800.00 mg, 21.94 mmol, 1 mL) was added. The resulting mixture was stirred at 25° C. for 15 hr. The reaction mixture was concentrated under reduce pressure and was then purified by reverse phase HPLC (10-10-40% 0-1-6 min H₂O/ACN/0.1% FA, flow: 30 mL/min (loading pump 4 mL/min ACN); column: Chromatorex 18 SNB100-5T 100*19 mm 5 um) to give N-(4-amino-1H-pyrazolo[4,3-c]pyridin-7-yl)-2-oxo-2-[rac-(2R,5S)-2-(6-methoxy-5-methyl-3-pyridyl)-5-methyl-1-piperidyl]acetamide (90 mg, 195.68 μmol, 72.72% yield, HCl).

¹H NMR (600 MHz, dmso) δ 0.67-1.07 (m, 3H), 1.20-1.42 (m, 1H), 1.51-2.25 (m, 7H), 2.74-3.07 (m, 1H), 3.66-4.09 (m, 4H), 4.97-5.69 (m, 1H), 6.93-7.23 (m, 3H), 7.45-7.55 (m, 1H), 7.67-7.79 (m, 1H), 7.80-7.99 (m, 1H), 8.15-8.45 (m, 1H), 9.54-10.70 (m, 1H), 12.69-13.56 (m, 1H).

LCMS(ESI): [M+H]⁺ m/z: calcd 424.2; found 424.2; Rt=2.534 min.

### Example 219. N-(4-Amino-2H-pyrazolo[4,3-c]pyridin-7-yl)-2-oxo-2-[rac-(2R,5S)-2-[2-(cyclopropoxy)-4-pyridyl]-5-methyl-1-piperidyl]acetamide (Compound 247)

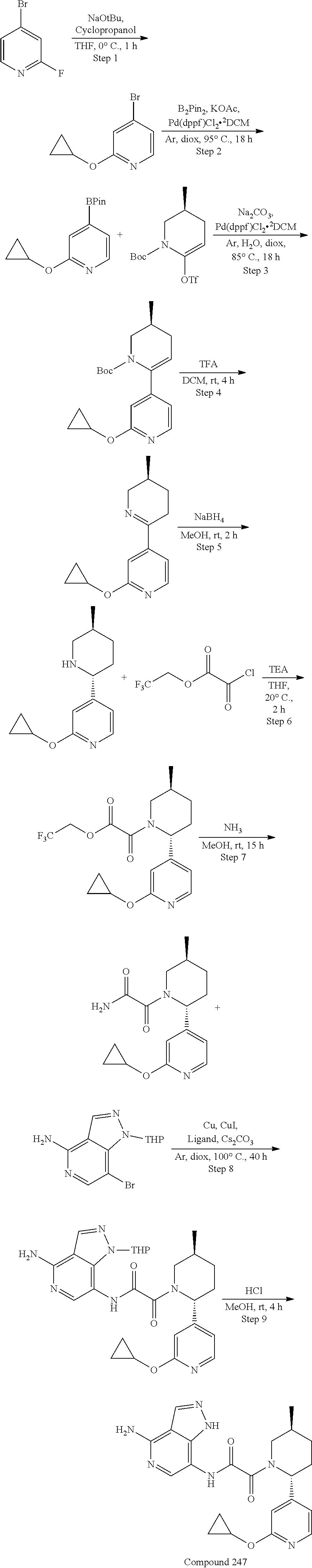

### Step 1: The Synthesis of 4-Bromo-2-(cyclopropoxy)pyridine

Sodium tert-butoxide (5.73 g, 59.66 mmol) was added to a stirred THF (70 mL), stirred for 10 min and cooled to 0° C. Solution of 4-bromo-2-fluoro-pyridine (7 g, 39.78 mmol, 4.09 mL) and, Cyclopropanol (3.70 g, 63.64 mmol) in THF (70 mL) was added to a stirred reaction mixture in 2 portions at 0° C., and stirred at the same temperature for 1 hr. Volatiles was evaporated, a residue mixed with water (20 mL) and extracted with TBME (2×20 mL), combined extract washed with brine (10 mL), dried over sodium sulphate, concentrated to give crude 4-bromo-2-(cyclopropoxy)pyridine (3.7 g, 17.28 mmol, 43.46% yield).

¹H NMR (500 MHz, CDCl₃) δ (ppm) 0.78 (m, 4H), 4.15 (m, 1H), 6.96 (s, 1H), 7.02 (d, 1H), 8.03 (d, 1H).

### Step 2: The Synthesis of 2-(Cyclopropoxy)-4-(4,4,5,5-tetramethyl-1,3,2-dioxaborolan-2-yl)pyridine

Potassium acetate (3.39 g, 34.57 mmol, 2.16 mL) was added to a solution of 4-bromo-2-(cyclopropoxy)pyridine (3.7 g, 17.28 mmol) and 4,4,5,5-tetramethyl-2-(4,4,5,5-tetramethyl-1,3,2-dioxaborolan-2-yl)-1,3,2-dioxaborolane (5.27 g, 20.74 mmol) in dioxane (80 mL). Reaction flask was evacuated and refilled with argon 3 times. Then cyclopentyl(diphenyl)phosphane; dichloromethane; dichloropalladium; iron (1.41 g, 1.73 mmol) added, degassed, refilled with argon 2 times and stirred at 95° C. for 18 hr to give 2-(cyclopropoxy)-4-(4,4,5,5-tetramethyl-1,3,2-dioxaborolan-2-yl)pyridine (4.5 g, 17.23 mmol, 99.70% yield). Reaction mixture 2-(cyclopropoxy)-4-(4,4,5,5-tetramethyl-1,3,2-dioxaborolan-2-yl)pyridine (4.5 g, 17.23 mmol, 99.70% yield).

### Step 3: The Synthesis of tert-Butyl rac-(3S)-6-[2-(cyclopropoxy)-4-pyridyl]-3-methyl-3,4-dihydro-2H-pyridine-1-carboxylate

Sodium carbonate (4.57 g, 43.08 mmol, 1.80 mL) was added to the solution of 2-(cyclopropoxy)-4-(4,4,5,5-tetramethyl-1,3,2-dioxaborolan-2-yl)pyridine (4.50 g, 17.23 mmol) and tert-butyl rac-(3S)-3-methyl-6-(trifluoromethylsulfonyloxy)-3,4-dihydro-2H-pyridine-1-carboxylate (6.55 g, 18.96 mmol) in Dioxane (60 mL) and Water (25 mL). Reaction flask was evacuated and refilled with argon 3 times. Then, PdCl₂*dppf*dcm (422.20 mg, 517.00 μmol) was added under stream of argon. Resulting mixture was stirred at 85° C. for 18 hr under inert atmosphere. Then, it was concentrated under reduced pressure and residue was extracted with ethyl acetate (100 mL). Obtained solution was filtered through a short pad of silicagel and evaporated under reduced pressure, affording tert-butyl rac-(3S)-6-[2-(cyclopropoxy)-4-pyridyl]-3-methyl-3,4-dihydro-2H-pyridine-1-carboxylate (8.5 g, crude).

LCMS(ESI): [M+H]⁺ m/z: calcd 331.2; found 331.2; Rt=1.584 min.

### Step 4: The Synthesis of 2-(Cyclopropoxy)-4-[rac-(3S)-3-methyl-2,3,4,5-tetrahydropyridin-6-yl]pyridine

Trifluoroacetic acid (29.33 g, 257.25 mmol, 19.82 mL) was added to the solution of tert-butyl rac-(3S)-6-[2-(cyclopropoxy)-4-pyridyl]-3-methyl-3,4-dihydro-2H-pyridine-1-carboxylate (8.5 g, 25.72 mmol) in Dichloromethane (40 mL). Resulting mixture was stirred at 25° C. for 4 hr. Then, volatiles were removed under reduced pressure and residue was taken up in water (60 mL). Insoluble tar material was filtered off through a cotton wool plug. Clear filtrate was basified with solid K₂CO₃ to pH≈10 and extracted with DCM (3×30 mL). Combined organic layers were dried over K₂CO₃ and concentrated in vacuo, affording 2-(cyclopropoxy)-4-[rac-(3S)-3-methyl-2,3,4,5-tetrahydropyridin-6-yl]pyridine (4.35 g, 18.89 mmol, 73.42% yield).

LCMS(ESI): [M+H]⁺ m/z: calcd 231.2; found 231.2; Rt=0.496 min.

### Step 5: The Synthesis of 2-(Cyclopropoxy)-4-[rac-(2R,5S)-5-methyl-2-piperidyl]pyridine

To the stirred solution of 2-(cyclopropoxy)-4-[rac-(3S)-3-methyl-2,3,4,5-tetrahydropyridin-6-yl]pyridine (4.35 g, 18.89 mmol) in Methanol (80 mL) was added Sodium borohydride (1.07 g, 28.33 mmol, 998.02 μL) in portions. Resulting mixture was stirred at 25° C. for 2 hr. Then, solvent was removed under reduced pressure and residue was partitioned between water (30 mL) and DCM (40 mL). Organic layer was separated, dried over K₂CO₃ and concentrated in vacuo, affording 2-(cyclopropoxy)-4-[rac-(2R,5S)-5-methyl-2-piperidyl]pyridine (2.71 g, 11.66 mmol, 61.76% yield).

LCMS(ESI): [M+H]⁺ m/z: calcd 233.2; found 233.2; Rt=0.774 min.

### Step 6: The Synthesis of 2,2,2-Trifluoroethyl 2-oxo-2-[rac-(2R,5S)-2-[2-(cyclopropoxy)-4-pyridyl]-5-methyl-1-piperidyl]acetate

2,2,2-Trifluoroethyl 2-chloro-2-oxo-acetate (2.89 g, 15.16 mmol) was added dropwise to a solution of 2-(cyclopropoxy)-4-[rac-(2R,5S)-5-methyl-2-piperidyl]pyridine (2.71 g, 11.66 mmol) and Triethylamine (1.89 g, 18.66 mmol, 2.60 mL) in Tetrahydrofuran (50 mL) at 0° C. After addition was complete, cooling bath was removed and resulting mixture was allowed to warm up to 20° C. and stirred for 2 hr. Then, resulting precipitate was filtered off and filtrate was concentrated under reduced pressure, leaving 2,2,2-trifluoroethyl 2-oxo-2-[rac-(2R,5S)-2-[2-(cyclopropoxy)-4-pyridyl]-5-methyl-1-piperidyl]acetate (5.1 g, crude).

LCMS(ESI): [M+H]⁺ m/z: calcd; found; Rt=min.

### Step 7: The Synthesis of 2-Oxo-2-[rac-(2R,5S)-2-[2-(cyclopropoxy)-4-pyridyl]-5-methyl-1-piperidyl]acetamide

2,2,2-Trifluoroethyl 2-oxo-2-[rac-(2R,5S)-2-[2-(cyclopropoxy)-4-pyridyl]-5-methyl-1-piperidyl]acetate (5.1 g, 9.24 mmol) was dissolved in Ammonia (7N in methanol) (23.37 g, 209.54 mmol, 30 mL, 15.27% purity). Resulting mixture was stirred at 25° C. for 15 hr. Then, volatiles were removed under reduced pressure and residue was purified by gradient column chromatography (SiO₂, MTBE/MeOH), affording 2-oxo-2-[rac-(2R,5S)-2-[2-(cyclopropoxy)-4-pyridyl]-5-methyl-1-piperidyl]acetamide (1.6 g, 5.27 mmol, 57.08% yield).

LCMS(ESI): [M+H]⁺ m/z: calcd 304.2; found 304.2; Rt=0.987 min.

### Step 8: The Synthesis of N-(4-Amino-2-tetrahydropyran-2-yl-pyrazolo[4,3-c]pyridin-7-yl)-2-oxo-2-[rac-(2R,5S)-2-[2-(cyclopropoxy)-4-pyridyl]-5-methyl-1-piperidyl]acetamide

2-Oxo-2-[rac-(2R,5S)-2-[2-(cyclopropoxy)-4-pyridyl]-5-methyl-1-piperidyl]acetamide (200 mg, 659.29 μmol), 7-bromo-2-tetrahydropyran-2-yl-pyrazolo[4,3-c]pyridin-4-amine (225.30 mg, 758.19 μmol), Copper (4.19 mg, 65.93 μmol), Copper (I) iodide (62.78 mg, 329.65 μmol, 11.17 μL), (S,S)—(+)—N,N′-Dimethyl-1,2-cyclohexanediamine (46.89 mg, 329.65 μmol) and Cesium carbonate (429.62 mg, 1.32 mmol) were mixed together in Dioxane (5 mL). Reaction flask was purged with argon and resulting mixture was stirred at 100° C. for 40 hr under inert atmosphere. Then, it was diluted with ethyl acetate (15 ml) and filtered. Filtrate was concentrated under reduced pressure, leaving N-(4-amino-2-tetrahydropyran-2-yl-pyrazolo[4,3-c]pyridin-7-yl)-2-oxo-2-[rac-(2R,5S)-2-[2-(cyclopropoxy)-4-pyridyl]-5-methyl-1-piperidyl]acetamide (410 mg, crude).

LCMS(ESI): [M+H]⁺ m/z: calcd 520.4; found 520.4; Rt=0.759 min.

### Step 9: The Synthesis of N-(4-Amino-2H-pyrazolo[4,3-c]pyridin-7-yl)-2-oxo-2-[rac-(2R,5S)-2-[2-(cyclopropoxy)-4-pyridyl]-5-methyl-1-piperidyl]acetamide (Compound 247)

Hydrochloric acid, 30% w/w aq. soln. (1.15 g, 9.46 mmol, 1 mL, 30% purity) was added to the solution of N-(4-amino-2-tetrahydropyran-2-yl-pyrazolo[4,3-c]pyridin-7-yl)-2-oxo-2-[rac-(2R,5S)-2-[2-(cyclopropoxy)-4-pyridyl]-5-methyl-1-piperidyl]acetamide (410 mg, 789.08 μmol) in Methanol (4 mL). Resulting mixture was stirring at 25° C. for 4 hr. Then, volatiles were removed and residue was subjected to HPLC (1-st run: 20-20-35% 0-1.5-5 min H₂O/ACN/0.1% NH₄OH, flow: 30 mL/min; column: YMC Triart C18 100×20 mm, 5 um; 2-nd run: 20-20-40% 0-1-5 min H₂O/ACN/0.1% FA, flow: 30 mL/min; column: Chromatorex 18 SMB100-5T 100×19 mm 5 um), affording N-(4-amino-2H-pyrazolo[4,3-c]pyridin-7-yl)-2-oxo-2-[rac-(2R,5S)-2-[2-(cyclopropoxy)-4-pyridyl]-5-methyl-1-piperidyl]acetamide (11.7 mg, 24.30 μmol, 3.08% yield, HCOOH).

¹H NMR (600 MHz, dmso) δ 0.15-0.77 (m, 4H), 0.80-1.03 (m, 3H), 1.17-1.43 (m, 1H), 1.53-2.29 (m, 4H), 2.73-3.14 (m, 1H), 3.73-4.12 (m, 1H), 4.19-4.36 (m, 1H), 4.93-5.70 (m, 1H), 6.47-7.55 (m, 4H), 7.60-8.22 (m, 3H), 9.54-10.64 (m, 1H), 12.61-13.42 (m, 1H).

LCMS(ESI): [M+H]⁺ m/z: calcd 436.2; found 436.2; Rt=1.495 min.

### Example 220. N-(4-Amino-1H-pyrazolo[4,3-c]pyridin-7-yl)-2-oxo-2-[rac-(2S,5R)-2-cyclobutyl-5-methyl-1-piperidyl]acetamide (Compound 249)

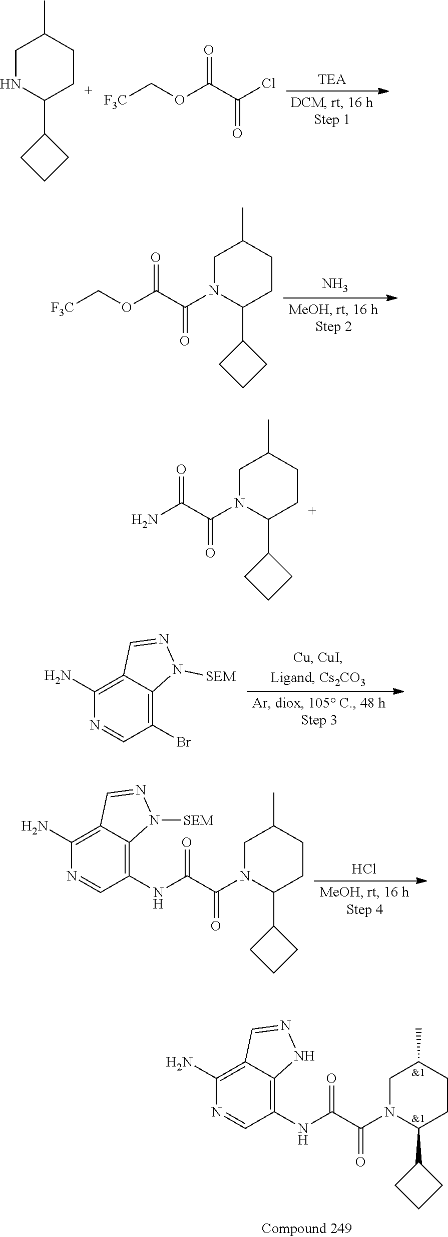

### Step 1: The Synthesis of 2,2,2-Trifluoroethyl 2-(2-cyclobutyl-5-methyl-1-piperidyl)-2-oxo-acetate

2-Cyclobutyl-5-methyl-piperidine (0.8 g, 5.22 mmol) was dissolved in DCM (18.63 mL) and TEA (1.32 g, 13.05 mmol, 1.82 mL) was added. Then reaction mixture was cooled and 2,2,2-trifluoroethyl 2-chloro-2-oxo-acetate (1.19 g, 6.26 mmol) was added dropwise. The reaction was stirred at rt overnight. After that water (50 ml) was added and organic layer was washed with brine (20 ml), dried over Na₂SO₄ and evaporated in vacuum to afford 2,2,2-trifluoroethyl 2-(2-cyclobutyl-5-methyl-1-piperidyl)-2-oxo-acetate (1.2 g, 3.90 mmol, 74.81% yield).

LCMS(ESI): [M+H]⁺ m/z: calcd 308.2; found; 308.2 Rt=1.459 min.

### Step 2: The Synthesis of 2-(2-cyclobutyl-5-methyl-1-piperidyl)-2-oxo-acetamide

2,2,2-Trifluoroethyl 2-(2-cyclobutyl-5-methyl-1-piperidyl)-2-oxo-acetate (1.2 g, 3.90 mmol) was dissolved in NH₃/MeOH (3.90 mmol, 10 mL) and stirred overnight at rt. Then it was evaporated in vacuum to afford 2-(2-cyclobutyl-5-methyl-1-piperidyl)-2-oxo-acetamide (0.87 g, 3.88 mmol, 99.33% yield).

LCMS(ESI): [M+H]⁺ m/z: calcd 225.2; found 225.2; Rt=1.045 min.

### Step 3: The Synthesis of N-[4-Amino-1-(2-trimethylsilylethoxymethyl)pyrazolo[4,3-c]pyridin-7-yl]-2-(2-cyclobutyl-5-methyl-1-piperidyl)-2-oxo-acetamide

A mixture of 7-bromo-1-(2-trimethylsilylethoxymethyl)pyrazolo[4,3-c]pyridin-4-amine (336.72 mg, 980.84 μmol), 2-(2-cyclobutyl-5-methyl-1-piperidyl)-2-oxo-acetamide (0.2 g, 891.67 μmol), Cesium carbonate (435.78 mg, 1.34 mmol), rac-(1R,2R)—N1,N2-dimethylcyclohexane-1,2-diamine (101.47 mg, 713.33 μmol) and CuI (101.89 mg, 535.00 μmol, 18.13 μL) with a few mg of Cu (2.83 mg, 44.58 μmol) in dioxane (3.98 mL) was stirred in a sealed vial under argon at 105° C. for 48 hr. The resulting mixture was cooled down and submitted to reverse phase HPLC (column: SunFire C18 100×19 mm, 5 um; mobile phase: 30-70% 2-10 min MeCN, flow rate: 30 mL/min) to afford N-[4-amino-1-(2-trimethylsilylethoxymethyl)pyrazolo[4,3-c]pyridin-7-yl]-2-(2-cyclobutyl-5-methyl-1-piperidyl)-2-oxo-acetamide (131.3 mg, 269.79 μmol, 30.26% yield).

LCMS(ESI): [M+H]⁺ m/z: calcd 487.2; found 487.2; Rt=1.207 min.

### Step 4: The Synthesis of N-(4-Amino-1H-pyrazolo[4,3-c]pyridin-7-yl)-2-oxo-2-[rac-(2S,5R)-2-cyclobutyl-5-methyl-1-piperidyl]acetamide (Compound 249)

N-[4-Amino-1-(2-trimethylsilylethoxymethyl)pyrazolo[4,3-c]pyridin-7-yl]-2-(2-cyclobutyl-5-methyl-1-piperidyl)-2-oxo-acetamide (131.3 mg, 269.79 μmol) was dissolved in MeOH (2 mL) and diox/HCl (269.79 μmol, 3 mL) was added. Then mixture was stirred at rt 16 hr. The solution was evaporated and submitted to reverse phase HPLC (column: SunFire C18 100×19 mm, 5 um; mobile phase: 0-50% 2-10 min MeCN+FA, flow rate: 30 mL/min) to afford N-(4-amino-1H-pyrazolo[4,3-c]pyridin-7-yl)-2-oxo-2-[rac-(2S,5R)-2-cyclobutyl-5-methyl-1-piperidyl]acetamide (46.3 mg, 115.05 μmol, 42.64% yield, HCOOH).

¹H NMR (600 MHz, dmso) δ 0.80-0.97 (m, 3H), 1.22-1.47 (m, 2H), 1.46-1.87 (m, 7H), 1.88-2.07 (m, 2H), 2.88-2.96 (m, 1H), 3.54-3.68 (m, 1H), 4.03-4.53 (m, 1H), 6.61-6.82 (m, 2H), 7.28-8.24 (m, 3H), 9.38-10.39 (m, 1H), 12.35-13.22 (m, 1H).

LCMS(ESI): [M+H]⁺ m/z: calcd 357.2; found 357.2; Rt=2.168 min.

### Example 221. N-(4-Amino-1H-pyrazolo[4,3-c]pyridin-7-yl)-2-oxo-2-[rac-(2S,5S)-5-methyl-2-(2-methylpyrazol-3-yl)-1-piperidyl]acetamide (Compound 250), and N-(4-Amino-1H-pyrazolo[4,3-c]pyridin-7-yl)-2-oxo-2-[rac-(2R,5R)-5-methyl-2-(2-methylpyrazol-3-yl)-1-piperidyl]acetamide (Compound 272)

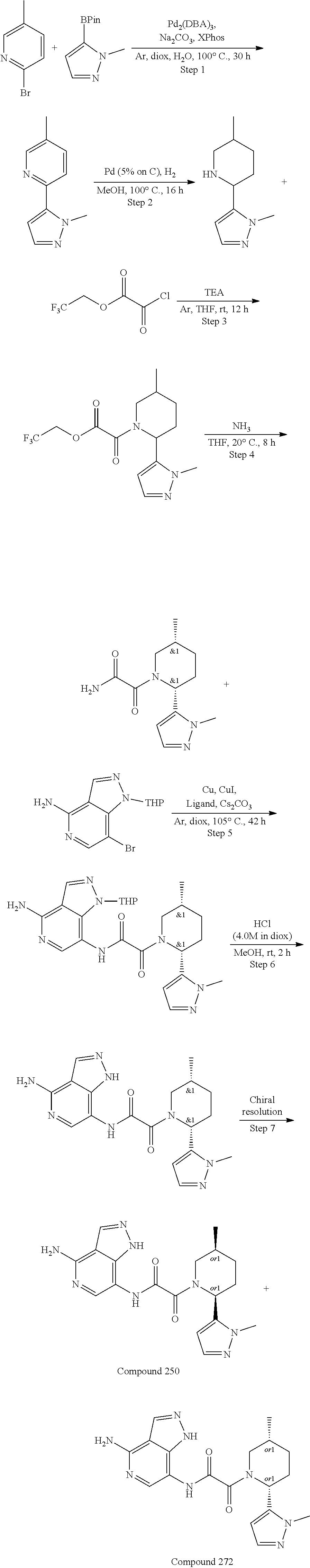

### Step 1: The Synthesis of 5-Methyl-2-(2-methylpyrazol-3-yl)pyridine

Pd₂(dba)₃ (1.86 g, 2.03 mmol) and XPhos (1.94 g, 4.07 mmol) was added to the solution of 2-bromo-5-methyl-pyridine (7 g, 40.69 mmol) and 1-methyl-5-(4,4,5,5-tetramethyl-1,3,2-dioxaborolan-2-yl) pyrazole (12.70 g, 61.04 mmol) in Dioxane (180 mL) and Water (10 mL). Reaction flask was evacuated and refilled with argon 3 times. Then, Sodium carbonate (8.63 g, 81.38 mmol, 3.41 mL) was added under stream of argon. Resulting mixture was stirred at 100° C. for 30 hr under inert atmosphere. Then, it was concentrated under reduced pressure and residue was extracted with boiling MTBE (120 ml). The combined organic extracts, dried over sodium sulphate and evaporated in vacuo to leave 15 g of crude product, 15 g of which was purification by column chromatography on silica gel using CHCl3/MTBE gradient (10-100% MTBE) to afford 5-methyl-2-(2-methylpyrazol-3-yl)pyridine (1.2 g, 6.93 mmol, 17.02% yield).

LCMS(ESI): [M+H]⁺ m/z: calcd 174.0; found 174.0; Rt=1.019 min.

### Step 2: The Synthesis of 5-Methyl-2-(2-methylpyrazol-3-yl)piperidine

5-Methyl-2-(2-methylpyrazol-3-yl)pyridine (1.2 g, 6.93 mmol), Platinum, 5% on carbon, dry (405.44 mg, 2.08 mmol) were dissolved in MeOH (20 mL). Reaction mixture was evacuated and refilled with hydrogen (50 atm) for 16 hr at 20° C. Catalyst was removed via filtration and volatiles were removed in vacuo to afford 5-methyl-2-(2-methylpyrazol-3-yl)piperidine (1 g, 5.58 mmol, 80.52% yield).

LCMS(ESI): [M+H]⁺ m/z: calcd 180.4; found 180.4; Rt=1.401 min.

### Step 3: The Synthesis of 2,2,2-Trifluoroethyl 2-[5-methyl-2-(2-methylpyrazol-3-yl)-1-piperidyl]-2-oxo-acetate

To a solution of 5-methyl-2-(2-methylpyrazol-3-yl)piperidine (1.4 g, 7.81 mmol) and TEA (1.19 g, 11.71 mmol, 1.63 mL) in THF (25.16 mL) was added 2,2,2-trifluoroethyl 2-chloro-2-oxo-acetate (1.79 g, 9.37 mmol) dropwise at 0° C. under argon. The reaction mixture was then stirred for 12 hr at r.t., then evaporated in vacuo to give 2,2,2-trifluoroethyl 2-[5-methyl-2-(2-methylpyrazol-3-yl)-1-piperidyl]-2-oxo-acetate (1.2 g, 3.60 mmol, 46.10% yield).

LCMS(ESI): [M+H]⁺ m/z: calcd 334.2; found 334.2; Rt=3.373 min.

### Step 4: The Synthesis of 2-Oxo-2-[rac-(2R,5R)-5-methyl-2-(2-methylpyrazol-3-yl)-1-piperidyl]acetamide

2,2,2-Trifluoroethyl 2-[5-methyl-2-(2-methylpyrazol-3-yl)-1-piperidyl]-2-oxo-acetate (1.2 g, 3.60 mmol) was dissolved in THF (20 mL) and was blow ammonium (1.30 g, 76.27 mmol). Resulting solution was stirred at 20° C. for 8 hr. The resulting mixture was evaporated in vacuo and residue was triturated with THF (20 ml*2), filtered and combined organic was evaporated in vacuo to leave 1 g of crude product and was purified by RP-HPLC (column: XBridge BEH18 SMB100-BT 100*19 mm; 20-20-45% 0-1-5 min H₂O/CH₃OH/0.1% NH₄OH, flow: 30 mL/min) to give 2-oxo-2-[rac-(2R,5R)-5-methyl-2-(2-methylpyrazol-3-yl)-1-piperidyl]acetamide (0.299 g, 1.19 mmol, 33.18% yield).

LCMS(ESI): [M+H]⁺ m/z: calcd 251.2; found 251.2; Rt=1.758 min.

### Step 5: The Synthesis of N-(4-Amino-2-tetrahydropyran-2-yl-pyrazolo[4,3-c]pyridin-7-yl)-2-oxo-2-[rac-(2R,5R)-5-methyl-2-(2-methylpyrazol-3-yl)-1-piperidyl]acetamide

A mixture of 2-oxo-2-[rac-(2R,5R)-5-methyl-2-(2-methylpyrazol-3-yl)-1-piperidyl]acetamide (299 mg, 1.19 mmol), 7-bromo-2-tetrahydropyran-2-yl-pyrazolo[4,3-c]pyridin-4-amine (567.95 mg, 1.91 mmol), copper (5 mg, 78.68 μmol), Copper (I) iodide (160 mg, 840.12 μmol, 28.47 μL), caesium carbonate (622.75 mg, 1.91 mmol) and rac-(1R,2R)—N1,N2-dimethylcyclohexane-1,2-diamine (160 mg, 1.12 mmol) in 1,4-dioxane (7 mL) was stirred in a sealed vial under argon at 105° C. for 42 hr. The resulting mixture was cooled down and filtered. The filtercake was washed successively with THF (3*5 mL) and dichloromethane (3*5 mL). The combined filtrate was concentrated in vacuo to afford crude N-(4-amino-2-tetrahydropyran-2-yl-pyrazolo[4,3-c]pyridin-7-yl)-2-oxo-2-[rac-(2R,5R)-5-methyl-2-(2-methylpyrazol-3-yl)-1-piperidyl]acetamide (1.4 g, crude) as brown gum, which was used directly in the next step.

LCMS(ESI): [M+H]⁺ m/z: calcd 467.2; found 467.2; Rt=2.363 min.

### Step 6: The Synthesis of N-(4-Amino-1H-pyrazolo[4,3-c]pyridin-7-yl)-2-oxo-2-[rac-(2R,5R)-5-methyl-2-(2-methylpyrazol-3-yl)-1-piperidyl]acetamide

Hydrogen chloride solution 4.0M in dioxane (8.40 g, 32.02 mmol, 8 mL, 13.9% purity) was added to a stirred solution of crude from previous step N-(4-amino-2-tetrahydropyran-2-yl-pyrazolo[4,3-c]pyridin-7-yl)-2-oxo-2-[rac-(2R,5R)-5-methyl-2-(2-methylpyrazol-3-yl)-1-piperidyl]acetamide (1.4 g, 3.00 mmol) in methanol (8 mL) at 25° C. The resulting solution was stirred at 25° C. for 1.5 hr, then concentrated to dryness in vacuo and the residue was submitted to reverse phase HPLC (column: Chromatorex 18 SMB100-5T 100×19 mm 5 um; mobile phase: 0-0-30% 0-2-5 min H₂O/Acetonitrile/0.2% FA; flow: 30 mL/min (loading pump 4 mL/min acetonitrile)) to afford N-(4-amino-1H-pyrazolo[4,3-c]pyridin-7-yl)-2-oxo-2-[rac-(2R,5R)-5-methyl-2-(2-methylpyrazol-3-yl)-1-piperidyl]acetamide (360 mg, 840.25 μmol, 28.00% yield, HCOOH) as beige solid. 1st fraction after HPLC (260 mg) was submitted to preparative chiral HPLC.

LCMS(ESI): [M+H]⁺ m/z: calcd 383.2; found 383.2; Rt=0.631 min.

### Step 7: The Synthesis of N-(4-Amino-1H-pyrazolo[4,3-c]pyridin-7-yl)-2-oxo-2-[rac-(2S,5S)-5-methyl-2-(2-methylpyrazol-3-yl)-1-piperidyl]acetamide (Compound 250) and N-(4-Amino-1H-pyrazolo[4,3-c]pyridin-7-yl)-2-oxo-2-[rac-(2R,5R)-5-methyl-2-(2-methylpyrazol-3-yl)-1-piperidyl]acetamide (Compound 272)

Racemic N-(4-amino-1H-pyrazolo[4,3-c]pyridin-7-yl)-2-oxo-2-[rac-(2R,5R)-5-methyl-2-(2-methylpyrazol-3-yl)-1-piperidyl]acetamide (260 mg, 606.85 μmol, HCOOH) was submitted to preparative chiral HPLC (Column: CHIRALPAK IC (250×30 mm, 10 mkm); Mobile phase: Hexane (0.1% DEA):IPA:MeOH, 50:25:25; Flow rate: 35 mL/min) to afford Compound 250 N-(4-amino-1H-pyrazolo[4,3-c]pyridin-7-yl)-2-oxo-2-[rac-(2S,5S)-5-methyl-2-(2-methylpyrazol-3-yl)-1-piperidyl]acetamide (134 mg, 350.40 μmol, 57.74% yield) (RetTime=14.427 min.) and Compound 272 N-(4-amino-1H-pyrazolo[4,3-c]pyridin-7-yl)-2-oxo-2-[rac-(2R,5R)-5-methyl-2-(2-methylpyrazol-3-yl)-1-piperidyl]acetamide (118 mg, 308.56 μmol, 50.85% yield) (RetTime=19.689 min).

### Compound 272

Preparative: RT (CHIRALPAK IC (250×30 mm, 10 mkm); Mobile Phase: Hexane (0.1% DEA):IPA:MeOH, 50:25:25; Flow Rate: 35 mL/min)=19.689 min.

Analytical: RT (Chiralpak IC (250×4.6 mm, 5 mkm)-; Mobile Phase: Hexane (0.1% EDA):IPA:MeOH, 50:25:2; Flow Rate: 0.6 mL/min)=22.278 min.

¹H NMR (600 MHZ, dmso) δ 0.73-0.89 (m, 3H), 1.34-1.71 (m, 2H), 1.73-1.94 (m, 2H), 2.06-2.31 (m, 1H), 2.61-2.95 (m, 1H), 3.46-4.34 (m, 4H), 5.32-5.87 (m, 1H), 5.99-6.50 (m, 1H), 6.54-7.27 (m, 2H), 7.32-7.50 (m, 1H), 7.58-8.44 (m, 2H), 9.58-10.66 (m, 1H), 12.32-13.78 (m, 1H).

LCMS(ESI): [M+H]⁺ m/z: calcd 383.2; found 383.0; Rt=1.972 min.

### Compound 250

Preparative: RT (CHIRALPAK IC (250×30 mm, 10 mkm); Mobile Phase: Hexane (0.1% DEA):IPA:MeOH, 50:25:25; Flow Rate: 35 mL/min)=14.427 min.

Analytical: RT (Chiralpak IC (250×4.6 mm, 5 mkm)-; Mobile Phase: Hexane (0.1% EDA):IPA:MeOH, 50:25:2; Flow Rate: 0.6 mL/min)=16.547 min.

¹H NMR (600 MHz, dmso) δ 0.69-0.92 (m, 3H), 1.32-1.96 (m, 4H), 2.07-2.30 (m, 1H), 2.60-2.95 (m, 1H), 3.48-4.25 (m, 4H), 5.33-5.93 (m, 1H), 6.02-6.51 (m, 1H), 6.63-7.48 (m, 3H), 7.55-8.43 (m, 2H), 9.59-10.68 (m, 1H), 12.55-13.52 (m, 1H).

LCMS(ESI): [M+H]⁺ m/z: calcd 383.2; found 383.2; Rt=1.685 min.

### Example 222. N-(4-Amino-1H-pyrazolo[4,3-c]pyridin-7-yl)-2-oxo-2-[rac-(2S)-2-[3-[ethyl(methyl)amino]phenyl]-1-piperidyl]acetamide (Compound 251) and N-(6-Amino-3-pyridyl)-2-[(2S,5R)-5-methyl-2-[2-(trifluoromethyl)-1,3-benzothiazol-5-yl]-1-piperidyl]-2-oxo-acetamide (Compound 267)

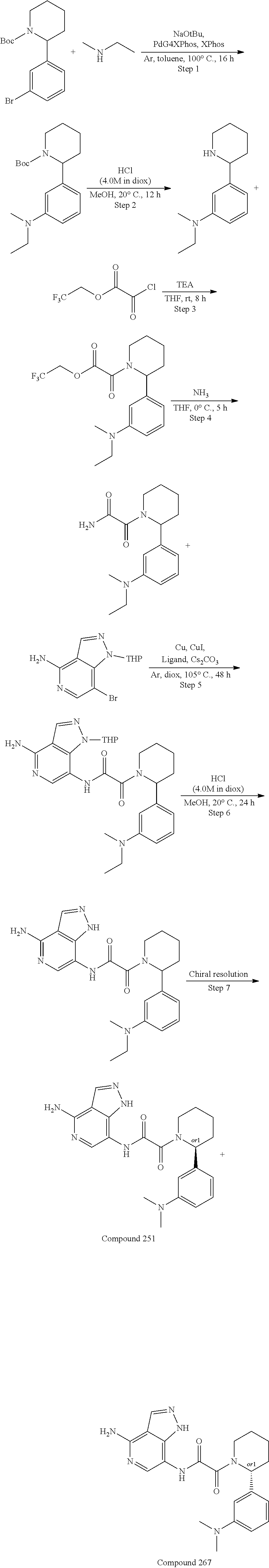

### Step 1: The Synthesis of tert-Butyl 2-[3-[ethyl(methyl)amino]phenyl]piperidine-1-carboxylate

tert-Butyl 2-(3-bromophenyl)piperidine-1-carboxylate (2 g, 5.88 mmol), N-methylethanamine (1.04 g, 17.63 mmol, 1.52 mL), XPhos (140.11 mg, 293.90 μmol) PdG4XPhos (252.89 mg, 293.90 μmol) and Sodium tert-butoxide (5.65 g, 11.76 mmol, 2 mL, 20% purity) were mixed in toluene (28.86 mL), purged with Ar for 15 minutes and then heated in the sealed tube at 100° C. for 16 hr. Final mixture was in vacuo and poured into water (40 ml) and extracted with DCM (2×40 ml). The combined organic extracts were washed with water (20 ml), dried over sodium sulphate and evaporated in vacuo to leave 2.15 g of crude product, 2.15 g of which was purification by column chromatography on silica gel using CHCl3/CH₃CN gradient (10-100% CH₃CN) to afford tert-butyl 2-[3-[ethyl(methyl)amino]phenyl]piperidine-1-carboxylate (1.2 g, 3.77 mmol, 64.11% yield).

### Step 2: The Synthesis of N-Ethyl-N-methyl-3-(2-piperidyl)aniline

Hydrogen chloride solution 4.0M in dioxane (8.00 g, 219.41 mmol, 10 mL) was added to a solution of tert-butyl 2-[3-[ethyl(methyl)amino]phenyl]piperidine-1-carboxylate (1.2 g, 3.77 mmol) in MeOH (30 mL). The reaction mixture was stirred at 20° C. for 12 hr, then evaporated and added to MTBE (15 ml) the resulting precipitate was filtered off, washed with MTBE (30 mL) and dried to afford N-ethyl-N-methyl-3-(2-piperidyl)aniline (1 g, 3.43 mmol, 91.11% yield, 2HCl).

### Step 3: The Synthesis of 2,2,2-Trifluoroethyl 2-[2-[3-[ethyl(methyl)amino]phenyl]-1-piperidyl]-2-oxo-acetate

To a solution of N-ethyl-N-methyl-3-(2-piperidyl)aniline (1 g, 3.43 mmol, 2HCl) and TEA (1.39 g, 13.73 mmol, 1.91 mL) in THF (29.99 mL) was added 2,2,2-trifluoroethyl 2-chloro-2-oxo-acetate (654.07 mg, 3.43 mmol) dropwise at 0° C. under argon. The reaction mixture was then stirred for 8 hr at r.t., then evaporated in vacuo to give 2,2,2-trifluoroethyl 2-[2-[3-[ethyl(methyl)amino]phenyl]-1-piperidyl]-2-oxo-acetate (1.28 g, 3.44 mmol, 100.00% yield).

### Step 4: The Synthesis of 2-[2-[3-[Ethyl(methyl)amino]phenyl]-1-piperidyl]-2-oxo-acetamide

2,2,2-Trifluoroethyl 2-[2-[3-[ethyl(methyl)amino]phenyl]-1-piperidyl]-2-oxo-acetate (1.28 g, 3.44 mmol) was dissolved in THF (40 mL) and was blow ammonium (1.24 g, 68.75 mmol). Resulting solution was stirred at 0° C. for 5 hr. The resulting mixture was evaporated in vacuo and residue was triturated with THF (20 mL*2), filtered and combined organic was evaporated in vacuo to leave 1.1 g of crude product, 1.1 g of which was purification by column chromatography on silica gel using CHCl₃/CH₃CN gradient (10-100% CH₃CN) to afford 2-[2-[3-[ethyl(methyl)amino]phenyl]-1-piperidyl]-2-oxo-acetamide (0.6 g, 2.07 mmol, 60.32% yield).

LCMS(ESI): [M+H]⁺ m/z: calcd 290.2; found 290.2; Rt=0.810 min.

### Step 5: The Synthesis of N-(4-Amino-1-tetrahydropyran-2-yl-pyrazolo[4,3-c]pyridin-7-yl)-2-[2-[3-[ethyl(methyl)amino]phenyl]-1-piperidyl]-2-oxo-acetamide

7-Bromo-1-tetrahydropyran-2-yl-pyrazolo[4,3-c]pyridin-4-amine (400.48 mg, 1.35 mmol), 2-[2-[3-[ethyl(methyl)amino]phenyl]-1-piperidyl]-2-oxo-acetamide (0.3 g, 1.04 mmol), Cu (3.29 mg, 51.84 μmol), CuI (184.17 mg, 967.01 μmol, 32.77 μL), cesium carbonate (506.68 mg, 1.56 mmol) and (1R,2R)—N1,N2-dimethylcyclohexane-1,2-diamine (184.17 mg, 1.29 mmol) were mixed in dioxane (5.99 mL), purged with Ar for 15 minutes and then heated in the sealed tube at 105° C. for 48 hr. Final mixture was filtered and dioxane was evaporated in vacuo. The crude product 0.6 g was purified by RP-HPLC (column: XBridge C18 5 um 130 A; 35-60% 0.5-6.5 min H₂O/CH₃CN/0.1% NH₄OH, flow: 30 mL/min) to give N-(4-amino-1-tetrahydropyran-2-yl-pyrazolo[4,3-c]pyridin-7-yl)-2-[2-[3-[ethyl(methyl)amino]phenyl]-1-piperidyl]-2-oxo-acetamide (0.1237 g, 244.65 μmol, 23.60% yield).

LCMS(ESI): [M+2H]⁺ m/z: calcd 507.2; found 507.2; Rt=0.952 min.

### Step 6: The Synthesis of N-(4-amino-1H-pyrazolo[4,3-c]pyridin-7-yl)-2-[2-[3-[ethyl(methyl)amino]phenyl]-1-piperidyl]-2-oxo-acetamide

Hydrogen chloride solution 4.0M in dioxane (1.21 g, 33.17 mmol, 1.51 mL) was added to a solution of N-(4-amino-1-tetrahydropyran-2-yl-pyrazolo[4,3-c]pyridin-7-yl)-2-[2-[3-[ethyl(methyl)amino]phenyl]-1-piperidyl]-2-oxo-acetamide (0.162 g, 320.40 μmol) in MeOH (4.49 mL). The reaction mixture was stirred at 20° C. for 24 hr, then evaporated was purified by RP-HPLC (column: XBridge BEH18 SMB100-BT 100*19 mm; 30-30-80% 0-1.3-5.3 min H₂O/CH₃OH/0.1% NH₄OH, flow: 30 mL/min) to give N-(4-amino-1H-pyrazolo[4,3-c]pyridin-7-yl)-2-[2-[3-[ethyl(methyl)amino]phenyl]-1-piperidyl]-2-oxo-acetamide (61.20 mg, 145.20 μmol, 45.32% yield).

LCMS(ESI): [M+H]⁺ m/z: calcd 422.2; found 422.2; Rt=1.422 min.

### Step 7: The Synthesis of N-(4-Amino-1H-pyrazolo[4,3-c]pyridin-7-yl)-2-oxo-2-[rac-(2S)-2-[3-[ethyl(methyl)amino]phenyl]-1-piperidyl]acetamide (Compound 251) and N-(6-Amino-3-pyridyl)-2-[(2S,5R)-5-methyl-2-[2-(trifluoromethyl)-1,3-benzothiazol-5-yl]-1-piperidyl]-2-oxo-acetamide (Compound 267)

The N-(4-amino-1H-pyrazolo[4,3-c]pyridin-7-yl)-2-[2-[3-[ethyl(methyl)amino]phenyl]-1-piperidyl]-2-oxo-acetamide (26.35 mg, 62.51 μmol) was subjected to chiral HPLC purification (Column: Chiralpak IJ (250-20 mm-5 m); Mobile phase: Hexane-MeOH-IPA-DEA, 50-25-25-0.1 Flow Rate: 10 mL/min) to give the two individual enantiomers Compound 251 N-(4-amino-1H-pyrazolo[4,3-c]pyridin-7-yl)-2-oxo-2-[rac-(2S)-2-[3-[ethyl(methyl)amino]phenyl]-1-piperidyl]acetamide (0.02518 g, 59.74 μmol, 95.56% yield) and Compound 267 N-(6-amino-3-pyridyl)-2-[(2S,5R)-5-methyl-2-[2-(trifluoromethyl)-1,3-benzothiazol-5-yl]-1-piperidyl]-2-oxo-acetamide (0.05775 g, 124.60 μmol, 50.22% yield).

### Compound 251:

Preparative: RT (Column: CHIRALCEL OJ-H (250×20 mm, 5 mkm); Mobile Phase: Hexane: IPA:MeOH:DEA, 50:25:25:0.1; Flow Rate: 12 mL/min)=14.702 min.

Analytical: RT (Column: Chiralcel OJ-H (250×4.6 mm, 5 mkm)-1; Mobile Phase: Hexane (0.1% EDA):IPA:MeOH, 50:25:25; Flow Rate: 0.6 mL/min)=14.094 min.

¹H NMR (600 MHz, dmso) δ 0.87-1.05 (m, 3H), 1.13-1.70 (m, 4H), 1.70-2.01 (m, 1H), 2.39-2.46 (m, 1H), 2.72-2.87 (m, 3H), 2.97-3.24 (m, 1H), 3.35-3.42 (m, 2H), 3.62-4.35 (m, 1H), 5.24-5.64 (m, 1H), 6.33-7.19 (m, 6H), 7.46-8.26 (m, 2H), 9.48-10.57 (m, 1H), 12.60-13.37 (m, 1H).

LCMS(ESI): [M+H]⁺ m/z: calcd 422.2; found 422.2; Rt=1.285 min.

### Compound 267:

Preparative: RT (Column: CHIRALCEL OJ-H (250×20 mm, 5 mkm); Mobile Phase: Hexane: IPA:MeOH:DEA, 50:25:25:0.1; Flow Rate: 12 mL/min)=28.988 min.

Analytical: RT (Column: Chiralcel OJ-H (250×4.6 mm, 5 mkm)-1; Mobile Phase: Hexane (0.1% EDA):IPA:MeOH, 50:25:25; Flow Rate: 0.6 mL/min)=29.567 min.

¹H NMR (600 MHz, dmso) δ 0.92-1.03 (m, 3H), 1.29-1.93 (m, 5H), 2.37-2.45 (m, 1H), 2.72-2.86 (m, 3H), 3.03-3.27 (m, 1H), 3.37 (q, 2H), 3.64-4.32 (m, 1H), 5.12-5.71 (m, 1H), 6.36-6.85 (m, 5H), 6.86-7.25 (m, 1H), 7.47-8.27 (m, 2H), 9.48-10.56 (m, 1H), 12.53-13.32 (m, 1H).

LCMS(ESI): [M+H]⁺ m/z: calcd 422.2; found 422.2; Rt=1.292 min.

### Example 223. N-(7-Amino-1-methyl-pyrazolo[3,4-c]pyridin-4-yl)-2-oxo-2-[(2R,5S)-5-methyl-2-[2-(1-methyl-4-piperidyl)-1,3-benzothiazol-5-yl]-1-piperidyl]acetamide (Compound 252)

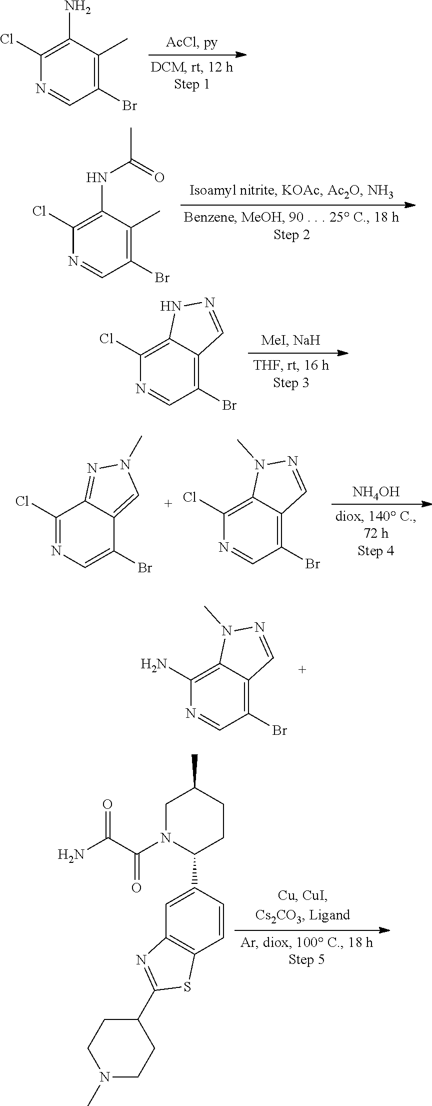

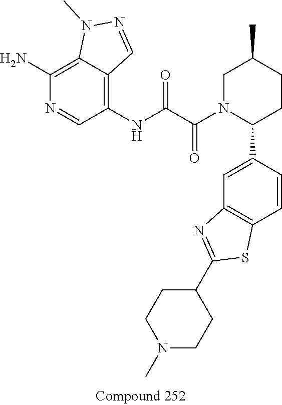 [SMILES: C[C@H]1CC[C@H](c2ccc3sc(C4CCN(C)CC4)nc3c2)N(C(=O)C(=O)Nc2cnc(N)c3c2cnn3C)C1]

The synthesis of 4-bromo-1-methyl-pyrazolo[3,4-c]pyridin-7-amine (Steps 1-4) was described previously in Compound 194.

### Step 5: The Synthesis of N-(7-Amino-1-methyl-pyrazolo[3,4-c]pyridin-4-yl)-2-oxo-2-[(2R,5S)-5-methyl-2-[2-(1-methyl-4-piperidyl)-1,3-benzothiazol-5-yl]-1-piperidyl]acetamide (Compound 252)

To a mixture of 4-bromo-1-methyl-pyrazolo[3,4-c]pyridin-7-amine (56.69 mg, 249.67 μmol), 2-oxo-2-[(2R,5S)-5-methyl-2-[2-(1-methyl-4-piperidyl)-1,3-benzothiazol-5-yl]-1-piperidyl]acetamide (0.11 g, 274.63 μmol), Copper (3.17 mg, 49.93 μmol), Copper (I) iodide (28.53 mg, 149.80 μmol, 5.08 μL), Cesium carbonate (97.62 mg, 299.60 μmol) and (1R,2R)—N1,N2-dimethylcyclohexane-1,2-diamine (21.31 mg, 149.80 μmol), Dioxane was added. The resulting mixture was evacuated, refiled with Argon three time, heated at 95° C. for 42 hr and cooled. The reaction mixture was subjected to HPLC ((Mobile Phase, Column): SYSTEM 30-30-80% 0-1.3-6.3 min H₂O/MeOH/0.1% NH₄OH, flow: 30 mL/min (loading pump 4 mL/min MeOH); column: XBridge BEH C18 5 um 130 A) and re-purified ((Mobile Phase, Column): SYSTEM 10-10-35% 0-1.3-6.3 min H₂O/ACN/0.1% NH₄OH, flow 30 mL/min (loading pump 4 mL/min ACN); column: XBridge BEH C18 5 um 130 A) to give N-(7-amino-1-methyl-pyrazolo[3,4-c]pyridin-4-yl)-2-oxo-2-[(2R,5S)-5-methyl-2-[2-(1-methyl-4-piperidyl)-1,3-benzothiazol-5-yl]-1-piperidyl]acetamide (11 mg, 20.12 μmol, 8.06% yield).

LCMS(ESI): [M+H]⁺ m/z: calcd 547.2; found 547.2; Rt=1.685 min.

### Example 224. N-(4-Amino-1H-pyrazolo[4,3-c]pyridin-7-yl)-2-oxo-2-[rac-(2R,5S)-2-(2-ethylindazol-6-yl)-5-methyl-1-piperidyl]acetamide (Compound 253)

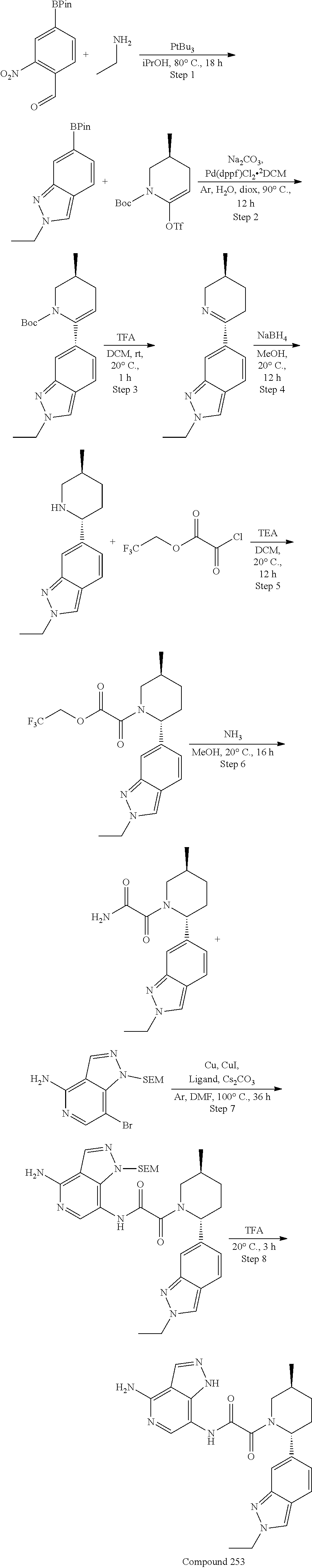

### Step 1: The Synthesis of 2-Ethyl-6-(4,4,5,5-tetramethyl-1,3,2-dioxaborolan-2-yl)indazole

2-Nitro-4-(4,4,5,5-tetramethyl-1,3,2-dioxaborolan-2-yl)benzaldehyde (5 g, 18.05 mmol) and ethanamine (1.63 g, 36.09 mmol, 2.03 mL) were dissolved in i-PrOH (53.04 mL) and the resulting solution was heated at 80° C. for 2 hr in a sealed tube. The reaction mixture was cooled to RT and Tri-n-butylphosphine, 95% (10.95 g, 54.14 mmol, 13.36 mL) was added, then the resulting mixture was heated at 80° C. overnight in a sealed tube. The reaction mixture was concentrated in vacuo. The crude product was purified by FCC (MTBE in hexanes from 16% to 50%). The reaction is successful. 2-Ethyl-6-(4,4,5,5-tetramethyl-1,3,2-dioxaborolan-2-yl)indazole (2 g, 7.35 mmol, 40.72% yield) was obtained as a brown gum.

LCMS(ESI): [M+H]⁺ m/z: calcd 273.2; found 273.2; Rt=1.128 min.

### Step 2: The Synthesis of tert-Butyl rac-(3S)-6-(2-ethylindazol-6-yl)-3-methyl-3,4-dihydro-2H-pyridine-1-carboxylate

2-Ethyl-6-(4,4,5,5-tetramethyl-1,3,2-dioxaborolan-2-yl)indazole (2 g, 7.35 mmol), tert-butyl rac-(3S)-3-methyl-6-(trifluoromethylsulfonyloxy)-3,4-dihydro-2H-pyridine-1-carboxylate (2.79 g, 8.08 mmol), Sodium carbonate (1.56 g, 14.70 mmol, 615.25 μL) and Pd(dppf)Cl₂·DCM (299.96 mg, 367.31 μmol) were mixed in dioxane (15 mL)+water (5 mL) under argon, and then stirred overnight at 90° C. for 12 hr in vial. The solid was filtered off, the filtrate was diluted with EA, the organic layer was separated, and the aqueous layer was extracted with additional EA, the combined organic layer was washed with brine, dried over Na₂SO₄, filtered and concentrated on vacuo. The obtained product was used in the next step without further purification. The reaction was successful. tert-Butyl rac-(3S)-6-(2-ethylindazol-6-yl)-3-methyl-3,4-dihydro-2H-pyridine-1-carboxylate (3.26 g, crude) was obtained as a red gum.

LCMS(ESI): [M+H]⁺ m/z: calcd 342.2; found 342.2; Rt=1.263 min.

### Step 3: The Synthesis of 2-Ethyl-6-[rac-(3S)-3-methyl-2,3,4,5-tetrahydropyridin-6-yl]indazole

tert-Butyl rac-(3S)-6-(2-ethylindazol-6-yl)-3-methyl-3,4-dihydro-2H-pyridine-1-carboxylate (3.26 g, 9.55 mmol) was dissolved in a mixture of DCM (15 mL) and TFA (15 mL). The resulting clear solution was stirred for 1 hr at 20° C. The reaction mixture was concentrated on vacuo. The obtained residue was dissolved in water and extracted with MTBE (3 times), after that the aqueous layer was basified with NaOH and extracted with DCM (3 times), combined DCM layers was dried over Na₂SO₄, filtered and evaporated. The reaction is successful. 2-Ethyl-6-[rac-(3S)-3-methyl-2,3,4,5-tetrahydropyridin-6-yl]indazole (1 g, 4.14 mmol, 43.40% yield) was obtained as a red solid.

### Step 4: The Synthesis of 2-Ethyl-6-[rac-(2R,5S)-5-methyl-2-piperidyl]indazole

To a stirring suspension of 2-ethyl-6-[rac-(3S)-3-methyl-2,3,4,5-tetrahydropyridin-6-yl]indazole (1 g, 4.14 mmol) in MeOH (20 mL) was added Sodium Borohydride (313.53 mg, 8.29 mmol, 291.93 μL) in portions. Resulting mixture was stirred at 20° C. for 12 hr. The solvent was removed under reduced pressure and residue was partitioned between water and DCM. The organic layer was separated, dried over Na₂SO₄ and concentrated in vacuo. The obtained product was used in the next step without further purification. The reaction is successful. 2-Ethyl-6-[rac-(2R,5S)-5-methyl-2-piperidyl]indazole (0.62 g, 2.55 mmol, 61.49% yield) was obtained as a brown gum.

LCMS(ESI): [M+H]⁺ m/z: calcd 244.2; found 244.2; Rt=0.597 min.

### Step 5: The Synthesis of 2,2,2-Trifluoroethyl 2-oxo-2-[rac-(2R,5S)-2-(2-ethylindazol-6-yl)-5-methyl-1-piperidyl]acetate

2,2,2-Trifluoroethyl 2-chloro-2-oxo-acetate (533.91 mg, 2.80 mmol, 508.48 μL) was added dropwise to a solution of 2-ethyl-6-[rac-(2R,5S)-5-methyl-2-piperidyl]indazole (0.62 g, 2.55 mmol) and TEA (296.48 mg, 2.93 mmol, 408.38 μL) in DCM (20 mL) at 0° C. After addition was complete, cooling bath was removed and resulting mixture was allowed to warm up to 20° C. and stirred for 12 hr. The reaction mixture was washed with Na₂CO₃, brine, dried over Na₂SO₄ and concentrated under reduced pressure. The obtained product was used in the next step without further purification. The reaction is successful. 2,2,2-Trifluoroethyl 2-oxo-2-[rac-(2R,5S)-2-(2-ethylindazol-6-yl)-5-methyl-1-piperidyl]acetate (1 g, 2.52 mmol, 98.77% yield) was obtained as a brown gum.

LCMS(ESI): [M+H]⁺ m/z: calcd 398.1; found 398.1; Rt=1.448 min.

### Step 6: The Synthesis of 2-Oxo-2-[rac-(2R,5S)-2-(2-ethylindazol-6-yl)-5-methyl-1-piperidyl]acetamide

2,2,2-Trifluoroethyl 2-oxo-2-[rac-(2R,5S)-2-(2-ethylindazol-6-yl)-5-methyl-1-piperidyl]acetate (1 g, 2.52 mmol) was dissolved in MeOH/NH₃ (20 mL) as stirred overnight at 20° C. The reaction mixture was evaporated to dryness. The obtained product was used in the next step without further purification. The reaction is successful. 2-Oxo-2-[rac-(2R,5S)-2-(2-ethylindazol-6-yl)-5-methyl-1-piperidyl]acetamide (0.8 g, crude) was obtained as a brown solid.

LCMS(ESI): [M+H]⁺ m/z: calcd 315.2; found 315.2; Rt=1.004 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-2-(2-ethylindazol-6-yl)-5-methyl-1-piperidyl]acetamide

2-Oxo-2-[rac-(2R,5S)-2-(2-ethylindazol-6-yl)-5-methyl-1-piperidyl]acetamide (0.2 g, 636.17 μmol), 7-bromo-1-(2-trimethylsilylethoxymethyl)pyrazolo[4,3-c]pyridin-4-amine (262.07 mg, 763.40 μmol), Copper (I) iodide (24.23 mg, 127.23 μmol, 4.31 μL), Cesium carbonate (414.55 mg, 1.27 mmol) and (1R,2R)—N1,N2-dimethylcyclohexane-1,2-diamine (108.59 mg, 763.40 μmol) were mixed in DMF (5 mL) under argon, and then stirred overnight at 100° C. for 36 hr in vial. The reaction mixture was submitted to HPLC (2-10 min 30-90% water-MeCN+FA; flow: 30 mL/min; loading pump 4 mL/min MeCN+FA; column SunFire 19*100 mm). The reaction was successful. N-[4-Amino-1-(2-trimethylsilylethoxymethyl)pyrazolo[4,3-c]pyridin-7-yl]-2-oxo-2-[rac-(2R,5S)-2-(2-ethylindazol-6-yl)-5-methyl-1-piperidyl]acetamide (0.1 g, 173.38 μmol, 27.25% yield) was obtained as a brown solid.

LCMS(ESI): [M+H]⁺ m/z: calcd 577.4; found 577.4; Rt=1.343 min.

### Step 8: The Synthesis of N-(4-Amino-1H-pyrazolo[4,3-c]pyridin-7-yl)-2-oxo-2-[rac-(2R,5S)-2-(2-ethylindazol-6-yl)-5-methyl-1-piperidyl]acetamide (Compound 253)

N-[4-Amino-1-(2-trimethylsilylethoxymethyl)pyrazolo[4,3-c]pyridin-7-yl]-2-oxo-2-[rac-(2R,5S)-2-(2-ethylindazol-6-yl)-5-methyl-1-piperidyl]acetamide (0.1 g, 173.38 μmol) was dissolved in trifluoroacetic acid (593.07 mg, 5.20 mmol, 400.72 μL) and stirred at 20° C. for 3 hr. The reaction mixture was submitted to HPLC (2-10 min 0-90% water-methanol+FA; flow: 30 mL/min; loading pump 4 mL/min methanol+FA; column SunFire 19*100 mm). The reaction is successful. N-(4-Amino-1H-pyrazolo[4,3-c]pyridin-7-yl)-2-oxo-2-[rac-(2R,5S)-2-(2-ethylindazol-6-yl)-5-methyl-1-piperidyl]acetamide (0.021 g, 37.46 μmol, 21.61% yield, CF₃COOH) was obtained as a light-yellow solid.

¹H NMR (600 MHz, dmso) δ 0.90-1.10 (m, 3H), 1.27-1.42 (m, 1H), 1.44-1.53 (m, 4H), 1.71-1.84 (m, 1H), 1.86-1.99 (m, 1H), 2.05-2.26 (m, 1H), 2.26-2.35 (m, 1H), 2.83-3.17 (m, 1H), 3.63-4.11 (m, 1H), 4.38-4.49 (m, 2H), 5.33-5.77 (m, 1H), 6.93-7.10 (m, 1H), 7.53 (s, 1H), 7.62-7.73 (m, 1H), 7.82-7.97 (m, 1H), 8.05-8.30 (m, 1H), 8.30-8.37 (m, 1H), 8.37-8.77 (m, 1H), 10.54-11.05 (m, 1H), 11.95-13.81 (m, 1H).

LCMS(ESI): [M+H]⁺ m/z: calcd 447.2; found 447.2; Rt=0.838 min.

### Example 225. N-(4-Amino-1H-pyrazolo[4,3-c]pyridin-7-yl)-2-oxo-2-[rac-(2R,5S)-2-tert-butyl-5-methyl-1-piperidyl]acetamide (Compound 254)

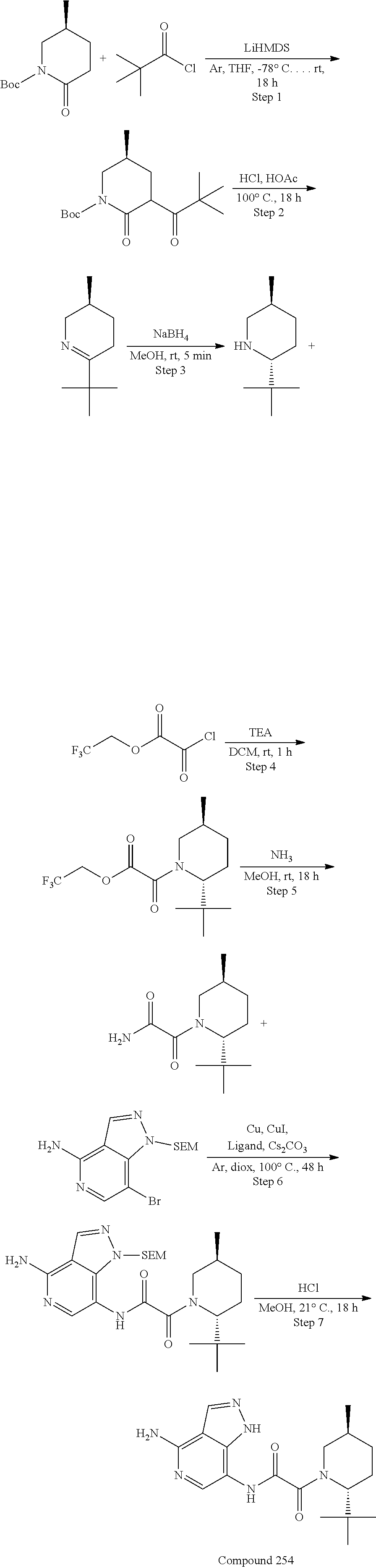

### Step 1: The Synthesis of tert-Butyl rac-(5S)-3-(2,2-dimethylpropanoyl)-5-methyl-2-oxo-piperidine-1-carboxylate

At −78° C., Lithium bis(trimethylsilyl)amide (8.24 g, 49.23 mmol, 49.23 mL) was added dropwise to a solution of tert-butyl rac-(5S)-5-methyl-2-oxo-piperidine-1-carboxylate (3 g, 14.07 mmol) in THF (50 mL) under Ar flow. The resulting mixture was stirred at −78° C. for 1 hr. Then the solution of 2,2-dimethylpropanoyl chloride (3.39 g, 28.13 mmol, 3.44 mL) in THF was added at −78° C. to the previous mixture. After 30 min the solution was allowed warm to rt and stirred overnight. The reaction mixture was quenched with NaHSO₄ (1 wt % solution) and extracted with DCM (3*50 mL). Organic layers were washed with water and dried over Na₂SO₄. DCM was evaporated to give tert-butyl rac-(5S)-3-(2,2-dimethylpropanoyl)-5-methyl-2-oxo-piperidine-1-carboxylate (5.42 g, crude).

¹H NMR (500 MHz, CDCl₃) δ (ppm) 1.02 (d, 3H), 1.18 (m, 16H), 1.49 (m, 12H), 1.96 (m, 2H), 2.64 (m, 1H), 3.18 (m, 1H), 3.78 (m, 1H), 4.12 (m, 1H),

### Step 2: The Synthesis of rac-(3S)-6-tert-Butyl-3-methyl-2,3,4,5-tetrahydropyridine

A solution of tert-butyl rac-(5S)-3-(2,2-dimethylpropanoyl)-5-methyl-2-oxo-piperidine-1-carboxylate (5.42 g, 18.23 mmol) in HOAc (25 mL) and 12N HCl (25 mL) was heated at 100° C. for 18 hr. The reaction mixture was warmed to rt and basified with 50% KOH aq. solution and then was left to stir overnight. This solution was extracted with DCM (3×20 mL). Organic phase was dried over Na₂SO₄, filtered and evaporated to obtain rac-(3S)-6-tert-butyl-3-methyl-2,3,4,5-tetrahydropyridine (2.79 g, 18.20 mmol, 100.00% yield) as a brown oil.

¹H NMR (400 MHZ, CDCl₃) δ (ppm) 0.88 (d, 3H), 1.08 (m, 10H), 1.71 (m, 2H), 2.08 (m, 1H), 2.32 (m, 1H), 2.97 (m, 1H), 3.74 (m, 1H).

### Step 3: The Synthesis of rac-(2R,5S)-2-tert-Butyl-5-methyl-piperidine

To an ice-cold solution of rac-(3S)-6-tert-butyl-3-methyl-2,3,4,5-tetrahydropyridine (2.8 g, 18.27 mmol) in MeOH (49.36 mL) was added sodium borohydride (691.12 mg, 18.27 mmol, 643.50 μL) in one portion and the resulting mixture was stirred for 5 min. The reaction mixture was evaporated, partitioned in mixture of DCM/water. Water was extracted with DCM (50 mL). Combined organics were dried over Na₂SO₄ and evaporated to give a residue that was used in the next step without further purification. rac-(2R,5S)-2-tert-Butyl-5-methyl-piperidine (1.5 g, crude) was obtained as a brown liquid.

### Step 4: The Synthesis of 2,2,2-Trifluoroethyl 2-oxo-2-[rac-(2R,5S)-2-tert-butyl-5-methyl-1-piperidyl]acetate

To a solution of rac-(2R,5S)-2-tert-butyl-5-methyl-piperidine (0.5 g, 3.22 mmol) and TEA (391.00 mg, 3.86 mmol, 538.56 μL) in DCM (25.27 mL) was added 2,2,2-trifluoroethyl 2-chloro-2-oxo-acetate (613.43 mg, 3.22 mmol) at rt. After stirring at rt for 1 hr the resulting mixture was washed with water, dried and evaporated to dryness to give 2,2,2-trifluoroethyl 2-oxo-2-[rac-(2R,5S)-2-tert-butyl-5-methyl-1-piperidyl]acetate (1 g, crude) as an off-white gum and was used in the next step without further purification.

LCMS(ESI): [M+H]⁺ m/z: calcd 310.2; found 310.2; Rt=4.313 min.

### Step 5: The Synthesis of 2-Oxo-2-[rac-(2R,5S)-2-tert-butyl-5-methyl-1-piperidyl]acetamide

Ammonia (55.06 mg, 3.23 mmol) was bubbled through a solution of 2,2,2-trifluoroethyl 2-oxo-2-[rac-(2R,5S)-2-tert-butyl-5-methyl-1-piperidyl]acetate (1 g, 3.23 mmol) in MeOH (25 mL) at rt. After stirring for 18 hr, the reaction mixture was evaporated to dryness and the residue was purified by CC (Interchim, 40 g SiO₂, hexane/MTBE (20-50-100%) flow rate=40 ml/min, RV=11.4-16.4 CV) to give 2-oxo-2-[rac-(2R,5S)-2-tert-butyl-5-methyl-1-piperidyl]acetamide (0.3 g, 1.33 mmol, 41.00% yield) as a yellow gum.

LCMS(ESI): [M+H]⁺ m/z: calcd 227.2; found 227.2; Rt=1.192 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-2-tert-butyl-5-methyl-1-piperidyl]acetamide

2-Oxo-2-[rac-(2R,5S)-2-tert-butyl-5-methyl-1-piperidyl]acetamide (0.15 g, 662.79 μmol), 7-bromo-1-(2-trimethylsilylethoxymethyl)pyrazolo[4,3-c]pyridin-4-amine (227.53 mg, 662.79 μmol), Copper (I) iodide (63.11 mg, 331.40 μmol, 11.23 μL), Cesium carbonate (431.90 mg, 1.33 mmol) and (1R,2R)—N1,N2-dimethylcyclohexane-1,2-diamine (94.28 mg, 662.79 μmol) were mixed in dioxane (5.00 mL) under argon, and then stirred for 72 h at 100° C. for 48 hr in vial. The reaction mixture was filtered and concentrated in vacuo. The residue was purified by HPLC (50-70% 2-6 min; flow 30 mL/min water-MeCN+NH₃ (loading pump 4 mL/min MeCN); column xbridge C18 19*100 mm (L)). N-[4-Amino-1-(2-trimethylsilylethoxymethyl)pyrazolo[4,3-c]pyridin-7-yl]-2-oxo-2-[rac-(2R,5S)-2-tert-butyl-5-methyl-1-piperidyl]acetamide (138.9 mg, 284.23 μmol, 42.88% yield) was obtained as a brown solid.

LCMS(ESI): [M+H]⁺ m/z: calcd 489.2; found 489.2; Rt=3.604 min.

### Step 7: The Synthesis of N-(4-Amino-1H-pyrazolo[4,3-c]pyridin-7-yl)-2-oxo-2-[rac-(2R,5S)-2-tert-butyl-5-methyl-1-piperidyl]acetamide (Compound 254)

To a solution of N-[4-amino-1-(2-trimethylsilylethoxymethyl)pyrazolo[4,3-c]pyridin-7-yl]-2-oxo-2-[rac-(2R,5S)-2-tert-butyl-5-methyl-1-piperidyl]acetamide (138.9 mg, 284.23 μmol) in MeOH (2 mL) was added Hydrogen chloride solution 4.0M in dioxane (1.60 g, 43.88 mmol, 2 mL) at 21° C. The resulting mixture was left to stir for 18 hr. The resulting mixture was evaporated to dryness and subjected to HPLC (20-35% 2-7 min; flow 30 mL/min water-ACN+NH₃ (loading pump 4 mL/min ACN); column XBRIDGE C18 19*100 mm (L)). N-(4-Amino-1H-pyrazolo[4,3-c]pyridin-7-yl)-2-oxo-2-[rac-(2R,5S)-2-tert-butyl-5-methyl-1-piperidyl]acetamide (70.9 mg, 197.80 μmol, 69.59% yield) was obtained as a beige solid.

¹H NMR (600 MHz, dmso) δ 0.86-0.97 (m, 12H), 1.53-1.63 (m, 1H), 1.63-1.75 (m, 1H), 1.76-1.96 (m, 2H), 2.99-3.29 (m, 1H), 3.43 (dd, 1H), 3.70 (d, 1H), 4.08-4.20 (m, 1H), 6.69 (d, 2H), 7.34-7.84 (m, 1H), 8.13 (d, 1H), 9.21-10.44 (m, 1H), 12.36-13.48 (m, 1H).

LCMS(ESI): [M+H]⁺ m/z: calcd 359.2; found 359.2; Rt=2.539 min.

### Example 226. N-(4-Amino-1H-pyrazolo[4,3-c]pyridin-7-yl)-2-oxo-2-[rac-(2R,5S)-2-(2,3-dihydrobenzofuran-7-yl)-5-methyl-1-piperidyl]acetamide (Compound 255)

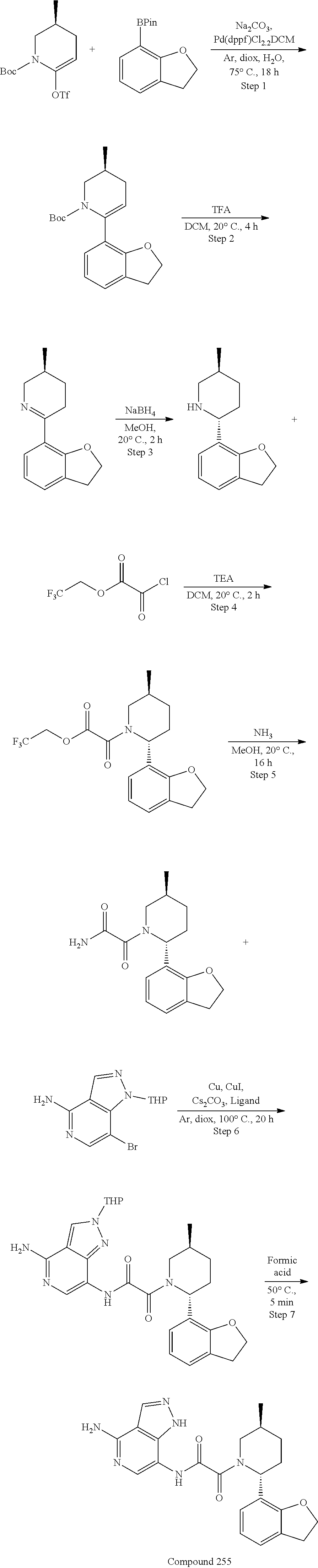

### Step 1: The Synthesis of tert-Butyl rac-(3S)-6-(2,3-dihydrobenzofuran-7-yl)-3-methyl-3,4-dihydro-2H-pyridine-1-carboxylate

Potassium carbonate (1.68 g, 12.19 mmol) was added to the solution of 2-(2,3-dihydrobenzofuran-7-yl)-4,4,5,5-tetramethyl-1,3,2-dioxaborolane (1.5 g, 6.09 mmol) and tert-butyl rac-(3S)-3-methyl-6-(trifluoromethylsulfonyloxy)-3,4-dihydro-2H-pyridine-1-carboxylate (2.53 g, 7.31 mmol) in the mixture of Dioxane (20 mL) and Water (8 mL). Reaction flask was evacuated and refilled with argon 3 times. Then, PdCl₂*dppf*dcm (149.32 mg, 182.85 μmol) was added under stream of argon. Resulting mixture was stirred at 75° C. for 18 hr under inert atmosphere. Then, it was concentrated under reduced pressure and residue was purified by gradient column chromatography (SiO₂, Hex/MTBE), affording tert-butyl rac-(3S)-6-(2,3-dihydrobenzofuran-7-yl)-3-methyl-3,4-dihydro-2H-pyridine-1-carboxylate (1.5 g, 4.76 mmol, 78.03% yield).

LCMS(ESI): [M+H]⁺ m/z: calcd 316.2; found 316.2; Rt=1.617 min.

### Step 2: The Synthesis of rac-(3S)-6-(2,3-Dihydrobenzofuran-7-yl)-3-methyl-2,3,4,5-tetrahydropyridine

Trifluoroacetic acid (5.59 g, 49.00 mmol, 3.75 mL) was added to the solution of tert-butyl rac-(3S)-6-(2,3-dihydrobenzofuran-7-yl)-3-methyl-3,4-dihydro-2H-pyridine-1-carboxylate (1.5 g, 4.76 mmol) in Dichloromethane (10 mL). Resulting mixture was stirred at 20° C. for 4 hr. Then, volatiles were removed under reduced pressure and residue was taken up in water (20 mL). Insoluble tar material was filtered off through a cotton wool plug. Clear filtrate was basified with solid K₂CO₃ to pH≈10 and extracted with DCM (2×15 mL). Combined organic layers were dried over K₂CO₃ and concentrated in vacuo, affording rac-(3S)-6-(2,3-dihydrobenzofuran-7-yl)-3-methyl-2,3,4,5-tetrahydropyridine (0.65 g, 3.02 mmol, 63.48% yield).

LCMS(ESI): [M+H]⁺ m/z: calcd 216.2; found 216.2; Rt=0.671 min.

### Step 3: The Synthesis of rac-(2R,5S)-2-(2,3-Dihydrobenzofuran-7-yl)-5-methyl-piperidine

To the stirred solution of rac-(3S)-6-(2,3-dihydrobenzofuran-7-yl)-3-methyl-2,3,4,5-tetrahydropyridine (0.65 g, 3.02 mmol) in Methanol (25 mL) was added Sodium borohydride (228.45 mg, 6.04 mmol, 212.71 μL) in portions. Resulting mixture was stirred at 20° C. for 2 hr. Then, solvent was removed under reduced pressure and residue was partitioned between water (30 mL) and DCM (30 mL). Organic layer was separated, dried over K₂CO₃ and concentrated in vacuo, affording rac-(2R,5S)-2-(2,3-dihydrobenzofuran-7-yl)-5-methyl-piperidine (0.64 g, 2.95 mmol, 97.55% yield).

LCMS(ESI): [M+H]⁺ m/z: calcd 218.2; found 218.2; Rt=0.703 min.

### Step 4: The Synthesis of 2,2,2-Trifluoroethyl 2-oxo-2-[rac-(2R,5S)-2-(2,3-dihydrobenzofuran-7-yl)-5-methyl-1-piperidyl]acetate

2,2,2-Trifluoroethyl 2-chloro-2-oxo-acetate (729.39 mg, 3.83 mmol) was added dropwise to the solution of rac-(2R,5S)-2-(2,3-dihydrobenzofuran-7-yl)-5-methyl-piperidine (0.64 g, 2.95 mmol) and Triethylamine (894.06 mg, 8.84 mmol, 1.23 mL) in Dichloromethane (19.96 mL) at 0° C. After addition was complete, cooling bath was removed and resulting mixture was allowed to warm up to 20° C. and stirred for 2 hr. Then, 10% aq. NaHCO₃ solution (20 mL) was added and stirring was continued for 5 min. After that, organic layer was separated, dried over Na₂SO₄ and concentrated under reduced pressure, affording 2,2,2-trifluoroethyl 2-oxo-2-[rac-(2R,5S)-2-(2,3-dihydrobenzofuran-7-yl)-5-methyl-1-piperidyl]acetate (1.1 g, crude).

LCMS(ESI): [M+H]⁺ m/z: calcd 372.2; found 372.2; Rt=1.396 min.

### Step 5: The Synthesis of 2-Oxo-2-[rac-(2R,5S)-2-(2,3-dihydrobenzofuran-7-yl)-5-methyl-1-piperidyl]acetamide

2,2,2-Trifluoroethyl 2-oxo-2-[rac-(2R,5S)-2-(2,3-dihydrobenzofuran-7-yl)-5-methyl-1-piperidyl]acetate (1.1 g, 2.96 mmol) was dissolved in Ammonia (7N in methanol, 15.3% w/w) (11.68 g, 104.98 mmol, 15 mL, 15.3% purity). Resulting reaction mixture was stirred at 20° C. for 16 hr. Then, volatiles were removed under reduced pressure and residue was purified by gradient column chromatography (SiO₂; CHCl₃/ACN), affording 2-oxo-2-[rac-(2R,5S)-2-(2,3-dihydrobenzofuran-7-yl)-5-methyl-1-piperidyl]acetamide (0.3 g, 1.04 mmol, 35.12% yield).

LCMS(ESI): [M+H]⁺ m/z: calcd 289.2; found 289.2; Rt=1.017 min.

### Step 6: The Synthesis of N-(4-Amino-2-tetrahydropyran-2-yl-pyrazolo[4,3-c]pyridin-7-yl)-2-oxo-2-[rac-(2R,5S)-2-(2,3-dihydrobenzofuran-7-yl)-5-methyl-1-piperidyl]acetamide

2-Oxo-2-[rac-(2R,5S)-2-(2,3-dihydrobenzofuran-7-yl)-5-methyl-1-piperidyl]acetamide (150.00 mg, 520.22 μmol), 7-bromo-2-tetrahydropyran-2-yl-pyrazolo[4,3-c]pyridin-4-amine (177.77 mg, 598.25 μmol), Copper (3.31 mg, 52.02 μmol), Copper (I) iodide (49.54 mg, 260.11 μmol, 8.81 μL), (S,S)—(+)—N,N′-Dimethyl-1,2-cyclohexanediamine (37.00 mg, 260.11 μmol, 41.02 μL) and Cesium carbonate (254.25 mg, 780.33 μmol) were mixed together in Dioxane (3 mL). Reaction flask was purged with argon and resulting mixture was stirred at 100° C. for 20 hr under inert atmosphere. Then, it was diluted with DCM (10 mL) and filtered. Filtrate was concentrated under reduced pressure, leaving N-(4-amino-2-tetrahydropyran-2-yl-pyrazolo[4,3-c]pyridin-7-yl)-2-oxo-2-[rac-(2R,5S)-2-(2,3-dihydrobenzofuran-7-yl)-5-methyl-1-piperidyl]acetamide (345 mg, crude).

LCMS(ESI): [M+H]⁺ m/z: calcd 505.2; found 505.2; Rt=1.061 min.

### Step 7: The Synthesis of N-(4-Amino-1H-pyrazolo[4,3-c]pyridin-7-yl)-2-oxo-2-[rac-(2R,5S)-2-(2,3-dihydrobenzofuran-7-yl)-5-methyl-1-piperidyl]acetamide (Compound 255)

N-(4-Amino-2-tetrahydropyran-2-yl-pyrazolo[4,3-c]pyridin-7-yl)-2-oxo-2-[rac-(2R,5S)-2-(2,3-dihydrobenzofuran-7-yl)-5-methyl-1-piperidyl]acetamide (170 mg, 336.91 μmol) was dissolved in Formic acid (2.44 g, 45.06 mmol, 2 mL, 85% purity). Resulting mixture was stirred at 50° C. for 5 min. Then, it was subjected to HPLC (1-st run: 10-10-60% 0-1-5 min H2O/ACN/0.2% FA, flow: 30 mL/min, column: Chromatorex 18 SMB100-5T 100×19 mm 5 um; 2-nd run: 10-10-45% 0-1-5 min H2O/ACN/0.1% NH4OH, flow: 30 mL/min, column: XBridge BEH C18 100×19 mm, 5 um), affording N-(4-amino-1H-pyrazolo[4,3-c]pyridin-7-yl)-2-oxo-2-[rac-(2R,5S)-2-(2,3-dihydrobenzofuran-7-yl)-5-methyl-1-piperidyl]acetamide (12 mg, 28.54 μmol, 8.47% yield).

¹H NMR (600 MHz, DMSO-d₆) ¿ (ppm) 0.83-1.04 (m, 3H), 1.17-1.35 (m, 1H), 1.56-2.16 (m, 4H), 3.04-3.18 (m, 2H), 3.48-4.64 (m, 4H), 5.09-5.62 (m, 1H), 6.55-7.19 (m, 5H), 7.36-7.79 (m, 1H), 8.07-8.28 (m, 1H), 9.45-10.60 (m, 1H), 12.43-13.46 (m, 1H).

LCMS(ESI): [M+H]⁺ m/z: calcd 421.2; found 421.2; Rt=2.256 min.

### Example 227. N-(4-Amino-1H-pyrazolo[4,3-c]pyridin-7-yl)-2-oxo-2-[rel-(2S)-2-[3-(dimethylamino)phenyl]-1-piperidyl]acetamide (Compound 283)) and N-(4-Amino-1H-pyrazolo[4,3-c]pyridin-7-yl)-2-oxo-2-[rel-(2R)-2-[3-(dimethylamino)phenyl]-1-piperidyl]acetamide (Compound 256)

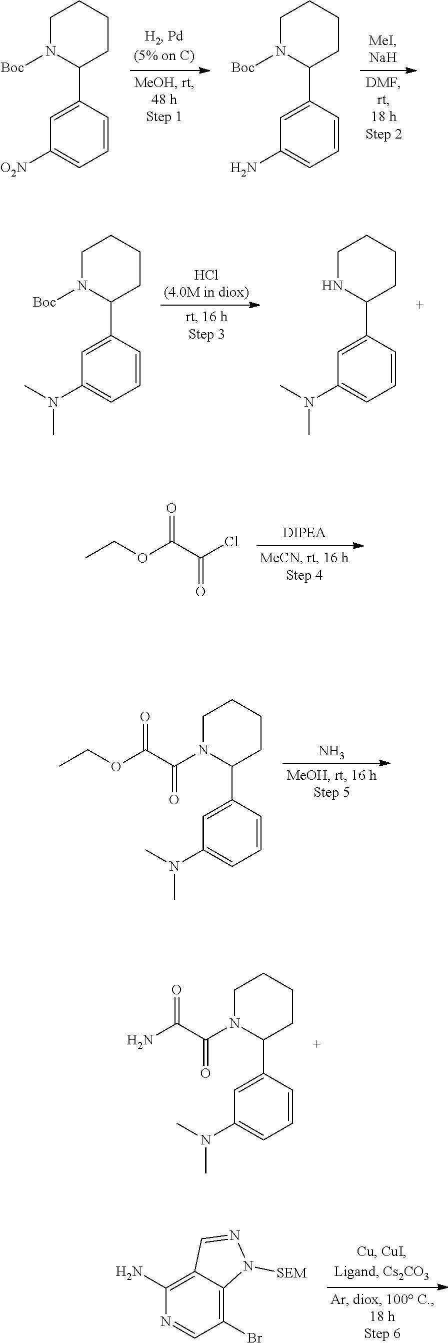

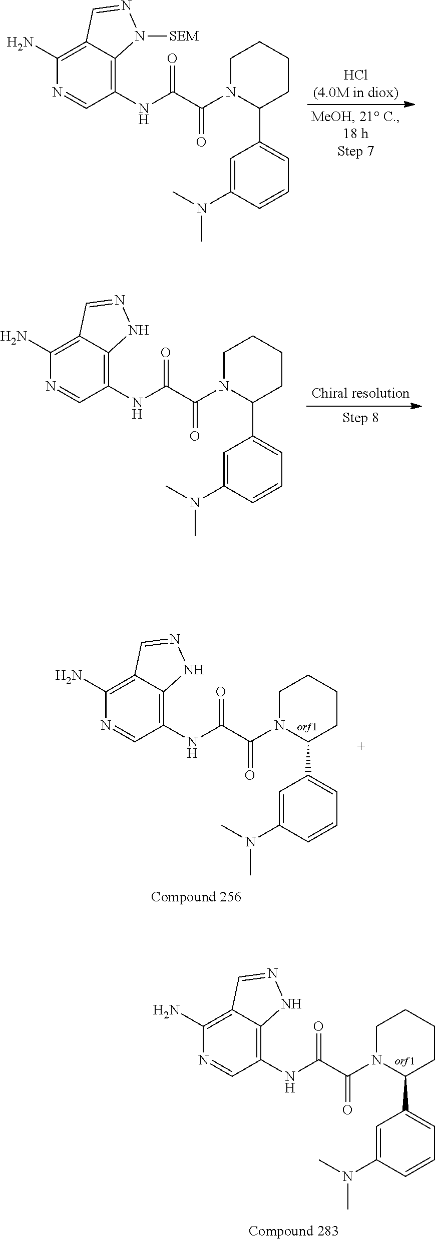 [SMILES: CN(C)c1cccc(C2CCCCN2C(=O)C(=O)Nc2cnc(N)c3cnn(COCC[Si](C)(C)C)c23)c1 ; "HCl(4.0M in diox)" ; CN(C)c1cccc(C2CCCCN2C(=O)C(=O)Nc2cnc(N)c3cn[nH]c23)c1 ; CN(C)c1cccc([C@H]2CCCCN2C(=O)C(=O)Nc2cnc(N)c3cn[nH]c23)c1 ; CN(C)c1cccc([C@@H]2CCCCN2C(=O)C(=O)Nc2cnc(N)c3cn[nH]c23)c1]

### Step 1: The Synthesis of tert-Butyl 2-(3-aminophenyl)piperidine-1-carboxylate

tert-Butyl 2-(3-nitrophenyl)piperidine-1-carboxylate (8 g, 26.11 mmol) was dissolved in methanol (150 mL) followed by addition of Platinum, 5% on carbon, dry (509.42 mg, 2.61 mmol). The reaction mixture was charged with Hydrogen and stirred in Hydrogen atmosphere for 48 hr. Upon completion, the reaction mixture was filtered and the filtrate was concentrated under reduced pressure to afford tert-butyl 2-(3-aminophenyl)piperidine-1-carboxylate (6.5 g, crude) which was used in the next step without purification.

LCMS(ESI): [M−tBu]⁺ m/z: calcd 221.0; found 221.0; Rt=1.202 min.

### Step 2: The Synthesis of tert-Butyl 2-[3-(dimethylamino)phenyl]piperidine-1-carboxylate

Sodium hydride (in oil dispersion) 60% dispersion in mineral oil (1.35 g, 58.80 mmol) was added portionwise to a cooled to 0° C. solution of tert-butyl 2-(3-aminophenyl)piperidine-1-carboxylate (6.5 g, 23.52 mmol) in DMFA (100 mL). The reaction mixture was stirred for 1 hr and then Iodomethane (6.68 g, 47.04 mmol, 2.93 mL) was added. The mixture was stirred at RT for 16 hr and concentrated under reduced pressure. The residue was dissolved in ethyl acetate and washed several times with water. The organic layer was separated, dried over Na₂SO₄ and concentrated on rotary evaporator to afford tert-butyl 2-[3-(dimethylamino)phenyl]piperidine-1-carboxylate (6 g, crude) which was used in the next step without purification.

LCMS(ESI): [M+H]⁺ m/z: calcd 305.2; found 305.2; Rt=1.301 min.

### Step 3: The Synthesis of N,N-Dimethyl-3-(2-piperidyl)aniline

tert-Butyl 2-[3-(dimethylamino)phenyl]piperidine-1-carboxylate (6 g, 19.71 mmol) was stirred in HCl/dioxane solution (13%, 150 mL) overnight. Upon completion, the reaction mixture was concentrated under reduced pressure to afford N,N-dimethyl-3-(2-piperidyl)aniline (6 g, crude, 2HCl) which was used in the next step without purification.

LCMS(ESI): [M+H]⁺ m/z: calcd 205.2; found 205.2; Rt=0.551 min.

### Step 4: The Synthesis of Ethyl 2-[2-[3-(dimethylamino)phenyl]-1-piperidyl]-2-oxo-acetate

N,N-Dimethyl-3-(2-piperidyl)aniline (6 g, 21.64 mmol, 2HCl) and DIPEA (11.19 g, 86.57 mmol, 15.08 mL) were dissolved in acetonitrile (86.63 mL). The reaction mixture was cooled to 0° C. and ethyl 2-chloro-2-oxo-acetate (2.95 g, 21.64 mmol, 2.42 mL) was dropwise added to the mixture. The reaction mixture stirred overnight, diluted with water and extracted with DCM. The organic layer was separated and concentrated under reduced pressure. The residue was submitted to flash column chromatography to afford ethyl 2-[2-[3-(dimethylamino)phenyl]-1-piperidyl]-2-oxo-acetate (1.3 g, 4.27 mmol, 19.73% yield).

### Chromatography Data:

Interchim; 80 g SiO₂, MTBE-MeOH from 0˜100%, flow rate=70 mL/min, cv=2.5

LCMS(ESI): [M+H]⁺ m/z: calcd 305.2; found 305.2; Rt=0.943 min.

### Step 5: The Synthesis of 2-[2-[3-(Dimethylamino)phenyl]-1-piperidyl]-2-oxo-acetamide

Ethyl 2-[2-[3-(dimethylamino)phenyl]-1-piperidyl]-2-oxo-acetate (1.3 g, 4.27 mmol) was dissolved in saturated ammonia/methanol solution (50 mL) and stirred overnight. Upon completion, the reaction mixture was concentrated under reduced pressure to afford 2-[2-[3-(dimethylamino)phenyl]-1-piperidyl]-2-oxo-acetamide (1 g, 3.63 mmol, 85.04% yield) which was used in the next step without purification.

LCMS(ESI): [M+H]⁺ m/z: calcd 276.2; found 276.2; Rt=0.850 min.

### Step 6: The Synthesis of N-[4-Amino-1-(2-trimethylsilylethoxymethyl)pyrazolo[4,3-c]pyridin-7-yl]-2-[2-[3-(dimethylamino)phenyl]-1-piperidyl]-2-oxo-acetamide

7-Bromo-1-(2-trimethylsilylethoxymethyl)pyrazolo[4,3-c]pyridin-4-amine (897.68 mg, 2.61 mmol), 2-[2-[3-(dimethylamino)phenyl]-1-piperidyl]-2-oxo-acetamide (720 mg, 2.61 mmol), Cu (17.61 mg, 277.18 μmol), CuI (149.40 mg, 784.47 μmol, 26.58 μL), Caesium carbonate (1.28 g, 3.92 mmol, 558.07 μL) and (1R,2R)—N1,N2-dimethylcyclohexane-1,2-diamine (111.58 mg, 784.47 μmol) were mixed in dioxane (4.66 mL), purged with Ar for 5 minutes and then heated in the sealed tube at 100° C. for 18 hr. Final mixture was filtered and dioxane was evaporated in vacuo. The crude product N-[4-amino-1-(2-trimethylsilylethoxymethyl)pyrazolo[4,3-c]pyridin-7-yl]-2-[2-[3-(dimethylamino)phenyl]-1-piperidyl]-2-oxo-acetamide (2 g, crude) as brown oil was used directly in the next step.

LCMS(ESI): [M+H]⁺ m/z: calcd 538.2; found 538.4; Rt=1.122 min.

### Step 7: The Synthesis of N-(4-Amino-1H-pyrazolo[4,3-c]pyridin-7-yl)-2-[2-[3-(dimethylamino)phenyl]-1-piperidyl]-2-oxo-acetamide

To a solution of N-[4-amino-1-(2-trimethylsilylethoxymethyl)pyrazolo[4,3-c]pyridin-7-yl]-2-[2-[3-(dimethylamino)phenyl]-1-piperidyl]-2-oxo-acetamide (2 g, 1.67 mmol) in methanol (10 mL) was added Hydrogen chloride solution 4.0M in dioxane (9.15 g, 25.11 mmol, 11.44 mL, 10% purity) at 21° C. The resulting mixture was left to stir for 18 hr. The resulting mixture was evaporated to dryness. The residue was purified by RP-HPLC (column: XBridge C18 100×19 mm, 5 um; 0-0-25% 0-1-5 min H₂O/MeCN/0.1% FA, flow: 30 ml/min, flow: 30 ml/min) to give N-(4-amino-1H-pyrazolo[4,3-c]pyridin-7-yl)-2-[2-[3-(dimethylamino)phenyl]-1-piperidyl]-2-oxo-acetamide (590 mg, 1.45 mmol, 86.51% yield).

LCMS(ESI): [M+H]⁺ m/z: calcd 408.2; found 408.2; Rt=1.089 min.

### Step 8: The Synthesis of N-(4-Amino-1H-pyrazolo[4,3-c]pyridin-7-yl)-2-oxo-2-[rac-(2S)-2-[3-(dimethylamino)phenyl]-1-piperidyl]acetamide (Compound 283) and N-(4-Amino-1H-pyrazolo[4,3-c]pyridin-7-yl)-2-oxo-2-[rac-(2R)-2-[3-(dimethylamino)phenyl]-1-piperidyl]acetamide (Compound 256)

The enantiomers were separated by chiral HPLC (Column: CHIRALCEL OJ-H (250×20 mm, 5 mkm); Mobile Phase: Hexane (0.1% EDA):IPA:MeOH, 50:25:25; Flow Rate: 15 ml/min) to give the two individual enantiomers Compound 283: N-(4-amino-1H-pyrazolo[4,3-c]pyridin-7-yl)-2-oxo-2-[rac-(2S)-2-[3-(dimethylamino)phenyl]-1-piperidyl]acetamide (144 mg, 353.40 μmol, 57.43% yield) and Compound 256: N-(4-amino-1H-pyrazolo[4,3-c]pyridin-7-yl)-2-oxo-2-[rac-(2R)-2-[3-(dimethylamino)phenyl]-1-piperidyl]acetamide (141 mg, 346.04 μmol, 56.23% yield).

### Compound 283:

Analytical: RT (Column: Chiralcel OJ-H (250×4.6 mm, 5 mkm); Mobile Phase: Hexane (0.1% EDA):IPA:MeOH, 50:25:25)=16.58 min;

Preparative: RT (Column: CHIRALCEL OJ-H (250×20 mm, 5 mkm); Mobile Phase: Hexane (0.1% EDA):IPA:MeOH, 50:25:25; Flow Rate: 15 ml/min)=16.0 min;
¹H NMR (600 MHz, dmso) δ 1.19-1.67 (m, 5H), 1.77-1.95 (m, 1H), 2.80-3.09 (m, 7H), 3.64-4.33 (m, 1H), 5.21-5.67 (m, 1H), 6.40-6.86 (m, 5H), 6.88-7.22 (m, 1H), 7.45-7.78 (m, 1H), 8.13-8.21 (m, 1H), 9.58-10.55 (m, 1H), 12.61-13.34 (m, 1H).

LCMS(ESI): [M+H]⁺ m/z: calcd 408.2; found 408.2; Rt=0.747 min.

### Compound 256:

Analytical: RT (Column: Chiralcel OJ-H (250×4.6 mm, 5 mkm); Mobile Phase: Hexane (0.1% EDA):IPA:MeOH, 50:25:25)=54.16 min.

Preparative: RT (Column: CHIRALCEL OJ-H (250×20 mm, 5 mkm); Mobile Phase: Hexane (0.1% EDA):IPA:MeOH, 50:25:25; Flow Rate: 15 ml/min)=46.4 min.

¹H NMR (600 MHz, dmso) δ 1.35-1.93 (m, 6H), 2.78-2.90 (m, 7H), 3.88-4.34 (m, 1H), 5.22-5.69 (m, 1H), 6.37-6.90 (m, 5H), 7.12-7.21 (m, 1H), 7.46-7.77 (m, 1H), 8.11-8.21 (m, 1H), 9.66-10.64 (m, 1H), 12.47-13.37 (m, 1H).

LCMS(ESI): [M+H]⁺ m/z: calcd 408.2; found 408.2; Rt=1.910 min.

### Example 228. N-(4-Amino-2H-pyrazolo[4,3-c]pyridin-7-yl)-2-oxo-2-[rac-(5S)-5-methyl-2-(1-methylindazol-4-yl)-1-piperidyl]acetamide (Compound 257)

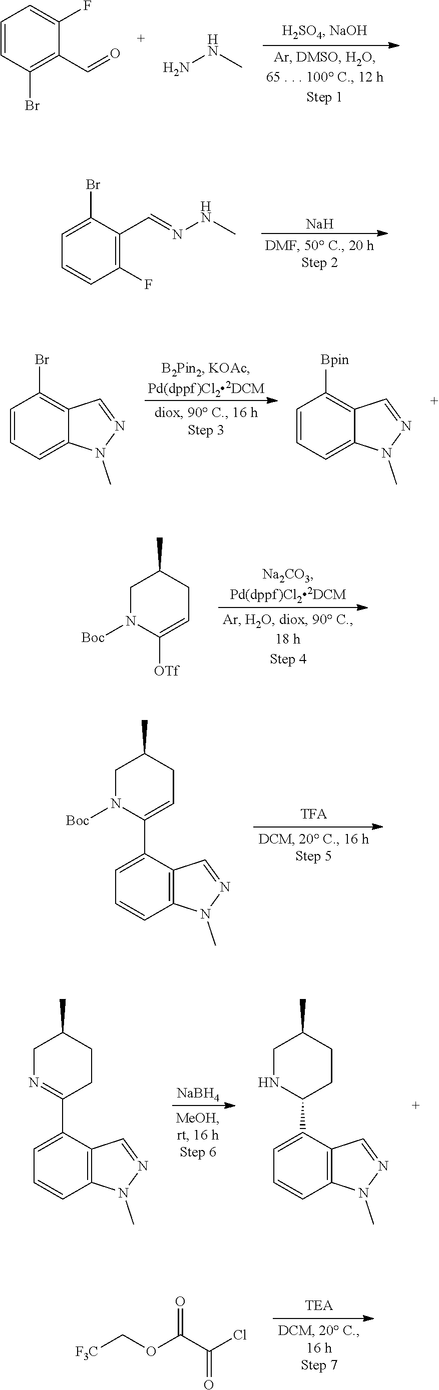

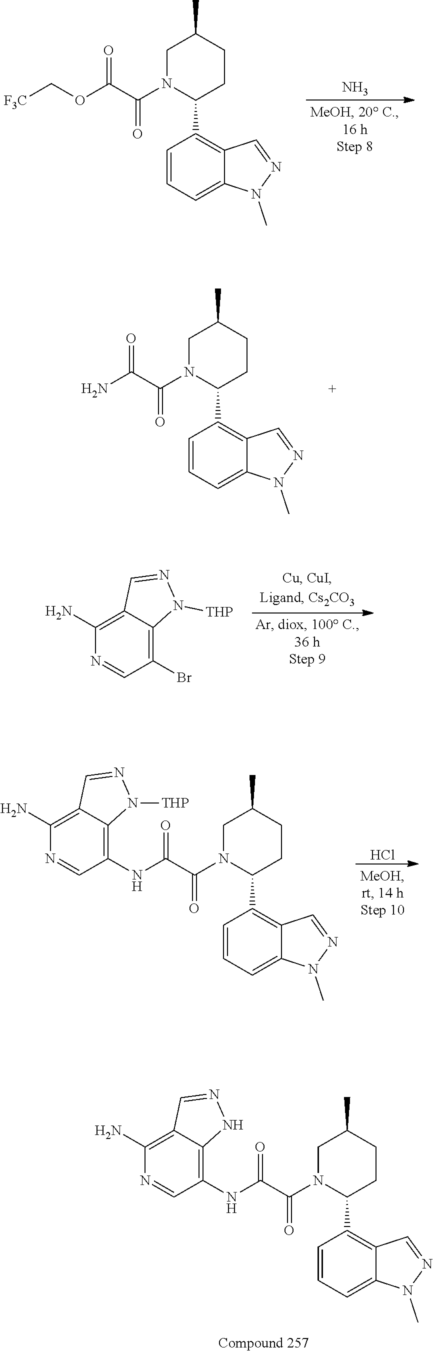

### Step 1: The Synthesis of N—[(E)-(2-Bromo-6-fluoro-phenyl)methyleneamino]methanamine

Sodium hydroxide (2.60 g, 65.02 mmol, 1.22 mL) was added to a stirred suspension of 2-bromo-6-fluoro-benzaldehyde (3 g, 14.78 mmol, 1.76 mL) and methylhydrazine; sulfuric acid (4.69 g, 32.51 mmol) in DMSO (35 mL) and Water (5 mL) mixture, stirred at 65° C. for 0.5 hr, at 80° C. for 0.5 hr and at 100° C. for 14 hr under Ar atmosphere. Reaction mixture was cooled to r.t., poured into water 200 mL, extracted with MTBE (3×50 mL), combined extract washed with water (3×100 mL), dried over sodium sulfate, concentrated in vacuo to give crude 4-bromo-1-methyl-indazole (3.2 g, crude) and N—[(E)-(2-bromo-6-fluoro-phenyl)methyleneamino]methanamine (3.2 g, crude).

LCMS(ESI): [M+H]⁺ m/z: calcd 211.0; found 211.0; Rt=0.853 min.

### Step 2: The Synthesis of 4-Bromo-1-methyl-indazole

To a stirred solution of N—[(E)-(2-bromo-6-fluoro-phenyl)methyleneamino]methanamine (3.2 g, 13.85 mmol) in DMF (25 mL) was added Sodium hydride (in oil dispersion) 60% dispersion in mineral oil (1.59 g, 41.55 mmol, 60% purity) at room temperature. The resulting reaction mixture was stirred at 50° C. for 20 hr. After 20 hours, the reaction mixture was quenched with water (100 mL). The aqueous phase was extracted with EtOAc (2×50 mL). The combined organic phase was washed with water (100 mL), brine (100 mL), dried over Na2SO4 and concentrated under reduced pressure. The residue was purified by FCC (Interchim; 40 g SiO₂, MTBE-MeOH from 0˜100%, flow rate=50 mL/min, cv=5) to obtain 4-bromo-1-methyl-indazole (1.3 g, 6.16 mmol, 44.48% yield).

LCMS(ESI): [M+H]⁺ m/z: calcd 210.0; found 210.0; Rt=1.243 min.

### Step 3: The Synthesis of 1-Methyl-4-(4,4,5,5-tetramethyl-1,3,2-dioxaborolan-2-yl)indazole

A solution of 4-bromo-1-methyl-indazole (1.3 g, 6.16 mmol), 4,4,5,5-tetramethyl-2-(4,4,5,5-tetramethyl-1,3,2-dioxaborolan-2-yl)-1,3,2-dioxaborolane (1.64 g, 6.47 mmol), Pd(dppf)Cl₂·DCM (251.50 mg, 307.97 μmol) and Potassium Acetate (1.81 g, 18.48 mmol, 1.16 mL) in Dioxane (20.00 mL) was heated at 90° C. for 16 hr. The reaction mixture was concentrated in vacuo and water (20 mL) was added thereto. The resulting mixture was extracted with EtOAc (2*10 mL) and combined organic layers were washed with brine, dried over Na₂SO₄, filtered and concentrated in vacuo to obtain 1-methyl-4-(4,4,5,5-tetramethyl-1,3,2-dioxaborolan-2-yl)indazole (1.6 g, crude).

LCMS(ESI): [M+H]⁺ m/z: calcd 259.2; found 259.2; Rt=1.434 min.

### Step 4: The Synthesis of tert-Butyl rac-(3S)-3-methyl-6-(1-methylindazol-4-yl)-3,4-dihydro-2H-pyridine-1-carboxylate

1-Methyl-4-(4,4,5,5-tetramethyl-1,3,2-dioxaborolan-2-yl)indazole (1.6 g, 6.20 mmol) and tert-butyl rac-(3S)-3-methyl-6-(trifluoromethylsulfonyloxy)-3,4-dihydro-2H-pyridine-1-carboxylate (2.14 g, 6.20 mmol) were dissolved in Dioxane (40 mL) and a solution of Sodium carbonate (1.31 g, 12.40 mmol, 518.95 μL) in Water (15 mL) was added thereto. The resulting mixture was evacuated and backfilled three times with argon. Pd(dppf)Cl2-CH2C12 (253.10 mg, 309.93 μmol) was added to the previous mixture and the resulting mixture was heated at 90° C. overnight. The reaction mixture was concentrated in vacuo and water (100 ml) was added to the residue. The resulting mixture was extracted with EtOAc (2*45 mL) and combined organic layers were washed with brine (25 ml), dried over Na₂SO₄, filtered and concentrated in vacuo to obtain tert-butyl rac-(3S)-3-methyl-6-(1-methylindazol-4-yl)-3,4-dihydro-2H-pyridine-1-carboxylate (1.8 g, crude).

LCMS(ESI): [M+H]⁺ m/z: calcd 328.2; found 328.2; Rt=1.371 min.

### Step 5: The Synthesis of 1-Methyl-4-[rac-(3S)-3-methyl-2,3,4,5-tetrahydropyridin-6-yl]indazole

Trifluoroacetic acid (14.80 g, 129.80 mmol, 10 mL) was added in one portion to a stirred solution of tert-butyl rac-(3S)-3-methyl-6-(1-methylindazol-4-yl)-3,4-dihydro-2H-pyridine-1-carboxylate (1.8 g, 5.50 mmol) in dichloromethane (20 mL). The resulting solution was stirred at 20° C. for 16 hr, and then concentrated in vacuo. The residue was diluted with water (50 mL). The resulting solution of TFA salt of the product was decanted from dark-brown oily residue, which was additionally rinsed with water (2*20 mL). The combined aqueous solution was filtered through a cotton pad to remove traces of oily impurities, then basified to pH 11-12 with 10% aqueous sodium carbonate solution and extracted with dichloromethane (2*40 mL). The combined organic extracts were dried over sodium sulfate and concentrated in vacuo to afford 1-methyl-4-[rac-(3S)-3-methyl-2,3,4,5-tetrahydropyridin-6-yl]indazole (565 mg, crude) which was directly used in the next step.

LCMS(ESI): [M+H]⁺ m/z: calcd 228.2; found 228.2; Rt=0.551 min.

### Step 6: The Synthesis of 1-Methyl-4-[rac-(5S)-5-methyl-2-piperidyl]indazole

1-Methyl-4-[rac-(3S)-3-methyl-2,3,4,5-tetrahydropyridin-6-yl]indazole (565 mg, 2.49 mmol) was dissolved in MeOH (15.00 mL) and Sodium Borohydride (282.10 mg, 7.46 mmol, 262.66 μL) was added portionwise. The resulting mixture was stirred overnight. Water (2 mL) was added to the reaction mixture and the resulting mixture was concentrated in vacuo. Water (30 mL) was added to the residue and the resulting mixture was extracted with DCM (2*30 mL). Combined organic layers were dried over Na₂SO₄, filtered and concentrated in vacuo to obtain 1-methyl-4-[rac-(5S)-5-methyl-2-piperidyl]indazole (0.45 g, crude).

LCMS(ESI): [M+H]⁺ m/z: calcd 230.2; found 230.2; Rt=0.806 min.

### Step 7: The Synthesis of 2,2,2-Trifluoroethyl 2-oxo-2-[rac-(5S)-5-methyl-2-(1-methylindazol-4-yl)-1-piperidyl]acetate

2,2,2-Trifluoroethyl 2-chloro-2-oxo-acetate (411.22 mg, 2.16 mmol) was added dropwise to a solution of 1-methyl-4-[rac-(5S)-5-methyl-2-piperidyl]indazole (0.45 g, 1.96 mmol) and triethylamine (238.28 mg, 2.35 mmol, 328.21 μL) in DCM (20 mL) at 0° C. After addition was complete, cooling bath was removed and resulting mixture was allowed to warm up to 20° C. and stirred for 16 hr. Then, it was washed with water, dried over Na₂SO₄ and concentrated under reduced pressure, affording 2,2,2-trifluoroethyl 2-oxo-2-[rac-(5S)-5-methyl-2-(1-methylindazol-4-yl)-1-piperidyl]acetate (0.6 g, 1.57 mmol, 79.76% yield) which was used in the next step without further purification.

LCMS(ESI): [M+H]⁺ m/z: calcd 384.2; found 384.2; Rt=1.401 min.

### Step 8: The Synthesis of 2-Oxo-2-[rac-(5S)-5-methyl-2-(1-methylindazol-4-yl)-1-piperidyl]acetamide

A solution of 2,2,2-trifluoroethyl 2-oxo-2-[rac-(5S)-5-methyl-2-(1-methylindazol-4-yl)-1-piperidyl]acetate (0.6 g, 1.57 mmol) in Methanol/NH₃ (5N) (10 mL) was stirred at 20° C. for 16 hr. The solvent was evaporated to obtain 2-oxo-2-[rac-(5S)-5-methyl-2-(1-methylindazol-4-yl)-1-piperidyl]acetamide (0.47 g, crude).

LCMS(ESI): [M+H]⁺ m/z: calcd 301.2; found 301.2; Rt=0.936 min.

### Step 9: The Synthesis of N-(4-Amino-2-tetrahydropyran-2-yl-pyrazolo[4,3-c]pyridin-7-yl)-2-oxo-2-[rac-(5S)-5-methyl-2-(1-methylindazol-4-yl)-1-piperidyl]acetamide

2-Oxo-2-[rac-(5S)-5-methyl-2-(1-methylindazol-4-yl)-1-piperidyl]acetamide (207 mg, 689.18 μmol), 7-bromo-2-tetrahydropyran-2-yl-pyrazolo[4,3-c]pyridin-4-amine (215.03 mg, 723.64 μmol), Copper (I) iodide (26.25 mg, 137.84 μmol, 4.67 μL), Cesium carbonate (449.10 mg, 1.38 mmol) and (1R,2R)—N1,N2-dimethylcyclohexane-1,2-diamine (117.64 mg, 827.02 μmol) were mixed in dioxane (6 mL) under argon, and then stirred overnight at 100° C. for 36 hr in vial. The reaction mixture was filtered and the filtrate was concentrated in vacuo and the residue was purified by HPLC (0-50% 2-10 min H₂O/ACN/0.1NH₄OH; flow 30 mL/min ((loading pump 4 mL ACN); column: Chromatorex C18 SMB100-5T 100*19 mm, 5 microM) to give N-(4-amino-2-tetrahydropyran-2-yl-pyrazolo[4,3-c]pyridin-7-yl)-2-oxo-2-[rac-(5S)-5-methyl-2-(1-methylindazol-4-yl)-1-piperidyl]acetamide (115.2 mg, 223.00 μmol, 32.36% yield).

LCMS(ESI): [M+H]⁺ m/z: calcd 517.0; found 517.0; Rt=1.159 min.

### Step 10: The Synthesis of N-(4-Amino-2H-pyrazolo[4,3-c]pyridin-7-yl)-2-oxo-2-[rac-(5S)-5-methyl-2-(1-methylindazol-4-yl)-1-piperidyl]acetamide (Compound 257)

To a solution of N-(4-amino-2-tetrahydropyran-2-yl-pyrazolo[4,3-c]pyridin-7-yl)-2-oxo-2-[rac-(5S)-5-methyl-2-(1-methylindazol-4-yl)-1-piperidyl]acetamide (115.2 mg, 223.00 μmol) in MeOH (3 mL) was added Hydrogen chloride solution 4.0M in dioxane (800.00 mg, 21.94 mmol, 1 mL) at 20° C. The resulting mixture was left to stirred for 14 hr. The resulting mixture was evaporated to dryness and then submitted to reverse phase HPLC (0-2-10 min 4 0-70% H₂O/MeOH/1.1NH₄OH, flow 30 mL/min ((loading pump 4 mL MeOH); column: Chromatorex C18 SMB100-5T 100*19 mm, 5 microM) to afford N-(4-amino-2H-pyrazolo[4,3-c]pyridin-7-yl)-2-oxo-2-[rac-(5S)-5-methyl-2-(1-methylindazol-4-yl)-1-piperidyl]acetamide (30.4 mg, 70.29 μmol, 31.52% yield).

¹H NMR (600 MHz, dmso) δ 0.88-1.09 (m, 3H), 1.26-1.43 (m, 1H), 1.69-1.97 (m, 2H), 2.18-2.24 (m, 1H), 2.91-3.26 (m, 1H), 3.36-3.59 (m, 1H), 3.78-4.15 (m, 4H), 5.34-5.98 (m, 1H), 6.55-6.92 (m, 2H), 6.92-7.16 (m, 1H), 7.32-7.43 (m, 1H), 7.44-7.79 (m, 2H), 7.86-8.28 (m, 2H), 9.44-10.71 (m, 1H), 12.46-13.54 (m, 1H).

LCMS(ESI): [M+H]⁺ m/z: calcd 433.2; found 433.2; Rt=0.573 min.

### Example 229. N-(4-Amino-1H-pyrazolo[4,3-c]pyridin-7-yl)-2-(3-methyl-2-phenyl-1-piperidyl)-2-oxo-acetamide (Compound 258)

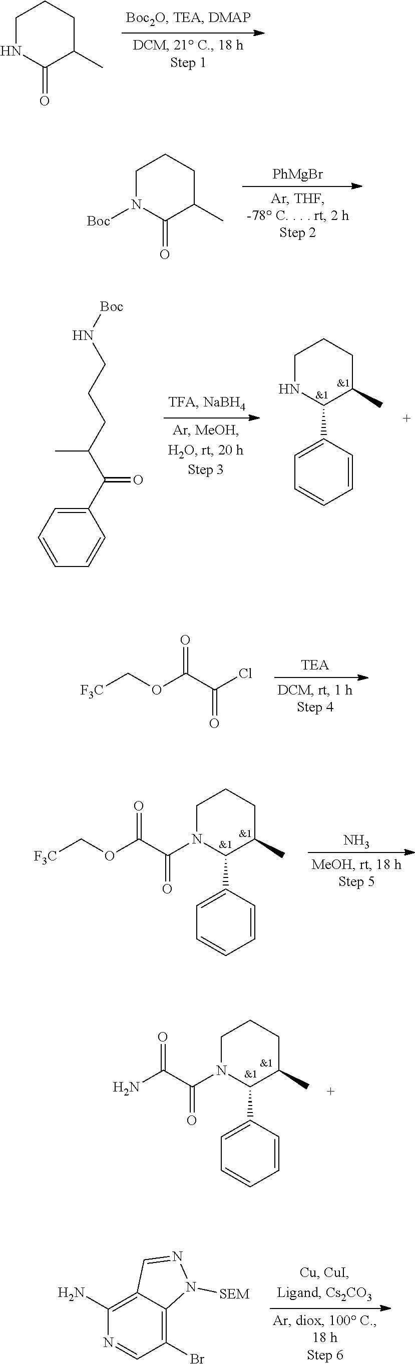

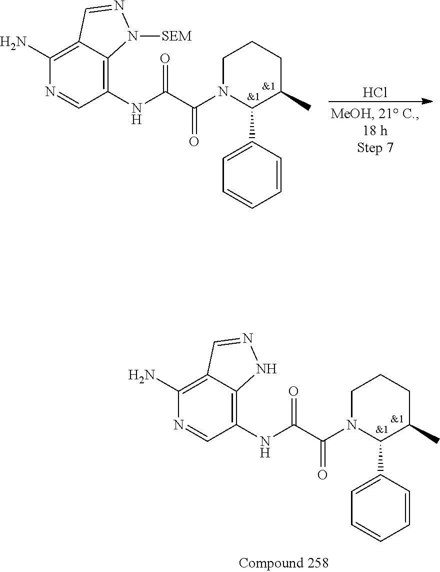 [SMILES: C[C@@H]1CCCN(C(=O)C(=O)Nc2cnc(N)c3cnn(COCC[Si](C)(C)C)c23)[C@H]1c1ccccc1 ; Cl ; C[C@@H]1CCCN(C(=O)C(=O)Nc2cnc(N)c3cn[nH]c23)[C@H]1c1ccccc1]

### Step 1: The Synthesis of tert-Butyl 3-methyl-2-oxo-piperidine-1-carboxylate

To a solution of 3-methylpiperidin-2-one (2 g, 17.67 mmol), Triethylamine (1.79 g, 17.67 mmol, 2.46 mL) and DMAP (215.93 mg, 1.77 mmol) in DCM (25 mL) was added di-tertbutyl dicarbonate (4.24 g, 19.44 mmol, 4.46 mL) portionwise at 21° C. The resulting solution was washed with 10% aq. HCl and brine, dried over Na₂SO₄ and evaporated to dryness to give tert-butyl 3-methyl-2-oxo-piperidine-1-carboxylate (3.5 g, 16.41 mmol, 92.85% yield) as a yellow oil.

LCMS(ESI): [M−tBu]⁺ m/z: calcd 158.2; found 158.2; Rt=1.172 min.

### Step 2: The Synthesis of tert-Butyl N-(4-methyl-5-oxo-5-phenyl-pentyl)carbamate

To a solution of tert-butyl 3-methyl-2-oxo-piperidine-1-carboxylate (3 g, 14.07 mmol) in THF (50 mL) at −78° C. was added phenylmagnesium bromide (17.00 g, 14.07 mmol, 17.35 mL) under Ar atmosphere. After addition was completed, the reaction mixture was allowed to warm to rt. The resulting mixture was quenched with aq NH₄Cl, extracted with EtOAc, dried over Na₂SO₄ and evaporated to give tert-butyl N-(4-methyl-5-oxo-5-phenyl-pentyl)carbamate (2.8 g, 9.61 mmol, 68.31% yield) as an orange gum, which was used in the next step without further purification.

LCMS(ESI): [M-Boc]⁺ m/z: calcd 192.2; found 192.2; Rt=1.453 min.

### Step 3: The Synthesis of 3-Methyl-2-phenylpiperidine

The tert-butyl N-(4-methyl-5-oxo-5-phenyl-pentyl)carbamate (2.8 g, 9.61 mmol) was stirred in Trifluoroacetic acid (14.80 g, 129.80 mmol, 10 mL) for 1 hr (until the gas evolution ceased). 50% w/v NaOH solution was added to the mixture until the pH was 13-14. The product was extracted 4×20 mL with DCM and the organic layers combined, dried with MgSO₄ and evaporated. The product was dissolved in mixture Methanol (40 mL)/water (10 mL) and added to a flask followed by Sodium Borohydride (399.90 mg, 10.57 mmol, 373.74 μL). The mixture was stirred under Ar overnight. The mixture was acidified with 1-2M HCl until the pH was 1-3 and left for 30 minutes. NaOH solution was then added until the pH was 13-14 and the product was extracted with DCM (4×100 mL), the organic layers were combined, dried with Na₂SO₄, filtered and evaporated. 3-methyl-2-phenylpiperidine (1.1 g, crude) was obtained as a pale yellow oil and was used in the next step without further purification.

LCMS(ESI): [M+H]⁺ m/z: calcd 176.2; found 176.2; Rt=0.751 min.

### Step 4: The Synthesis of 2,2,2-Trifluoroethyl 2-(3-methyl-2-phenyl-1-piperidyl)-2-oxo-acetate

To a solution of 3-methyl-2-phenyl-piperidine (335 mg, 1.91 mmol) and TEA (232.09 mg, 2.29 mmol, 319.68 μL) in DCM (25 mL) was added 2,2,2-trifluoroethyl 2-chloro-2-oxo-acetate (364.12 mg, 1.91 mmol) at rt. After stirring at rt for 1 hr the resulting mixture was washed with water, dried, and evaporated to dryness to give 2,2,2-trifluoroethyl 2-(3-methyl-2-phenyl-1-piperidyl)-2-oxo-acetate (600 mg, crude) as an yellow gum and was used in the next step without further purification.

LCMS(ESI): [M+H]⁺ m/z: calcd 330.2; found 330.2; Rt=3.664 min.

### Step 5: The Synthesis of 2-(3-Methyl-2-phenyl-1-piperidyl)-2-oxo-acetamide

Ammonia (31.03 mg, 1.82 mmol) was bubbled through a solution of 2,2,2-trifluoroethyl 2-(3-methyl-2-phenyl-1-piperidyl)-2-oxo-acetate (600 mg, 1.82 mmol) in MeOH (50 mL) at rt. After stirring for 18 hr, the reaction mixture was evaporated to dryness and purified by HPLC (25-50% 0.5-6.5 min; 30 mL/min water-acetonitrile+NH₃ (loading pump 4 mL/min; acetonitrile) affording 2-(3-methyl-2-phenyl-1-piperidyl)-2-oxo-acetamide (213.2 mg, 47.53% yield).

LCMS(ESI): [M+H]⁺ m/z: calcd 247.2; found 247.2; Rt=1.085 min.

### Step 6: The Synthesis of N-[4-Amino-1-(2-trimethylsilylethoxymethyl)pyrazolo[4,3-c]pyridin-7-yl]-2-(3-methyl-2-phenyl-1-piperidyl)-2-oxo-acetamide

2-(3-Methyl-2-phenyl-1-piperidyl)-2-oxo-acetamide (161.8 mg, 459.84 μmol), 7-bromo-1-(2-trimethylsilylethoxymethyl)pyrazolo[4,3-c]pyridin-4-amine (157.86 mg, 459.84 μmol), Copper (I) iodide (43.79 mg, 229.92 μmol, 7.79 μL), Cesium carbonate (299.65 mg, 919.68 μmol) and (1R,2R)—N1,N2-dimethylcyclohexane-1,2-diamine (65.41 mg, 459.84 μmol) were mixed in dioxane (5 mL) under argon, and then stirred overnight at 100° C. for 18 hr in vial. The reaction mixture was filtered and the filtrate was concentrated in vacuo and the residue was purified by HPLC (40-65% 0.5-6.5 min; 30 mL/min water-acetonitrile+NH₃ (loading pump 4 mL/min acetonitrile); column SunFire C18 19*100 mm) to give N-[4-amino-1-(2-trimethylsilylethoxymethyl)pyrazolo[4,3-c]pyridin-7-yl]-2-(3-methyl-2-phenyl-1-piperidyl)-2-oxo-acetamide (99.9 mg, 196.39 μmol, 42.71% yield) as a beige solid.
1st fr.—16.8 mg (97% by LCMS)
2nd fr.—83.1 mg (100% by LCMS)

LCMS(ESI): [M+H]⁺ m/z: calcd 509.2; found 509.2; Rt=1.450 min.

### Step 7: The Synthesis of N-(4-Amino-1H-pyrazolo[4,3-c]pyridin-7-yl)-2-(3-methyl-2-phenyl-1-piperidyl)-2-oxo-acetamide (Compound 258)

To a solution of N-[4-amino-1-(2-trimethylsilylethoxymethyl)pyrazolo[4,3-c]pyridin-7-yl]-2-(3-methyl-2-phenyl-1-piperidyl)-2-oxo-acetamide (83.1 mg, 163.36 μmol) in MeOH (2 mL) was added Hydrogen chloride solution 4.0M in dioxane (1.60 g, 43.88 mmol, 2 mL) at 21° C. The resulting mixture was left to stir for 18 hr. The resulting mixture was evaporated to dryness and subjected to HPLC (30-55% 0.5-8 min; 30 mL/min water-acetonitrile+NH₃ (loading pump 4 mL/min acn); column xbridgeC18 19*100 mm).
1st fr.—11.8 mg (100% by LCMS, ˜1:1 mixture of isomers)
2nd fr.—37.2 mg (100% by LCMS, ˜1:1 mixture of isomers)

¹H NMR (600 MHz, dmso) δ 0.71-1.22 (m, 3H), 1.36-1.49 (m, 1H), 1.58-1.78 (m, 2H), 1.79-2.10 (m, 1H), 2.57-2.73 (m, 1H), 2.99-3.10 (m, 0.5H), 3.38-3.46 (m, 0.5H), 3.63-4.32 (m, 1H), 4.99-5.51 (m, 1H), 7.14-7.30 (m, 1H), 7.35-7.41 (m, 2H), 7.41-7.54 (m, 1H), 7.80-7.98 (m, 1H), 7.98-9.14 (m, 3H), 10.66-10.98 (m, 1H), 12.62 (br s, 1H), 13.74 (br s, 1H).

LCMS(ESI): [M+H]⁺ m/z: calcd 379.2; found 379.2; Rt=2.146 min.

### Example 230. N-(4-Amino-1H-pyrazolo[4,3-c]pyridin-7-yl)-2-oxo-2-[rac-(2R,5S)-5-methyl-2-[5-(trifluoromethyl)-3-pyridyl]-1-piperidyl]acetamide (Compound 260)

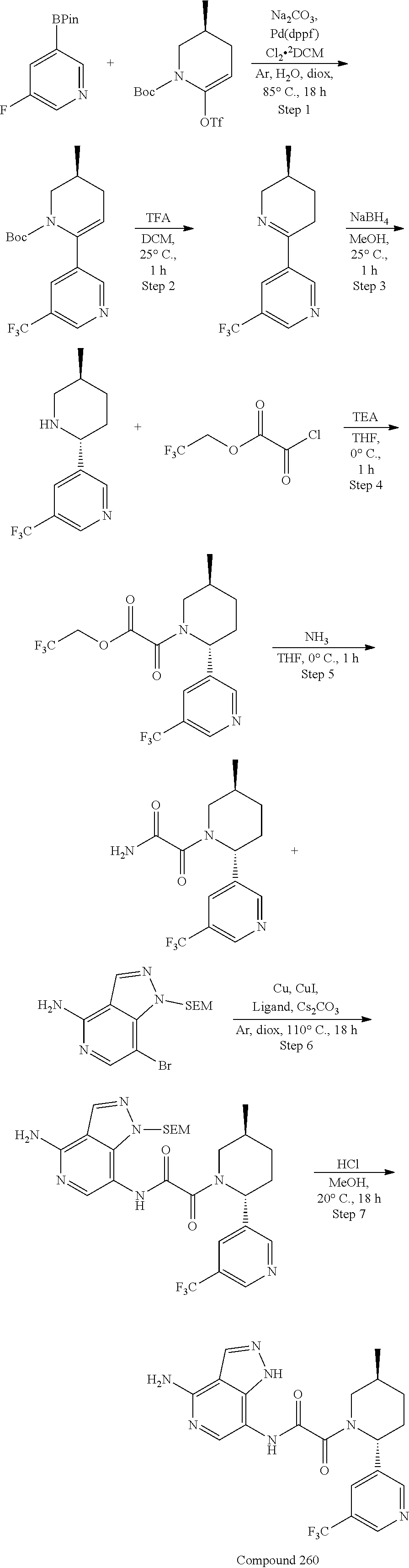

### Step 1: The Synthesis of tert-Butyl rac-(3S)-3-methyl-6-[5-(trifluoromethyl)-3-pyridyl]-3,4-dihydro-2H-pyridine-1-carboxylate

3-(4,4,5,5-Tetramethyl-1,3,2-dioxaborolan-2-yl)-5-(trifluoromethyl)pyridine (3.5 g, 12.82 mmol), tert-butyl rac-(3S)-3-methyl-6-(trifluoromethylsulfonyloxy)-3,4-dihydro-2H-pyridine-1-carboxylate (5.7 g, 16.51 mmol) and Sodium carbonate (3.7 g, 34.91 mmol, 1.46 mL) were added to a mixture of 1,4-dioxane (100 mL) and water (12 mL). The resulting mixture was evacuated and then backfilled with argon, this operation was repeated three times, then Pd(dppf)Cl₂·DCM (523.37 mg, 640.89 μmol) was added under argon. The reaction mixture was stirred under argon at 85° C. for 18 hr. The reaction mixture was cooled down and concentrated in vacuo. The residue was diluted with MTBE (120 mL) and sodium sulfate was added to remove residual water. The resulting mixture was stirred for 10 min, and then filtered. The filtercake was additionally washed with MTBE (2*25 mL) and discarded. The filtrate was concentrated in vacuo to afford crude tert-butyl rac-(3S)-3-methyl-6-[5-(trifluoromethyl)-3-pyridyl]-3,4-dihydro-2H-pyridine-1-carboxylate (8.8 g, crude) as red gum, which was used directly in the next step.

LCMS(ESI): [M+H]⁺ m/z: calcd 343.2; found 343.2; Rt=1.326 min.

### Step 2: The Synthesis of 3-[rac-(3S)-3-Methyl-2,3,4,5-tetrahydropyridin-6-yl]-5-(trifluoromethyl)pyridine

Trifluoroacetic acid (37.00 g, 324.50 mmol, 25 mL) was added in one portion to a stirred solution of tert-butyl rac-(3S)-3-methyl-6-[5-(trifluoromethyl)-3-pyridyl]-3,4-dihydro-2H-pyridine-1-carboxylate (8.8 g, 25.70 mmol) in dichloromethane (15 mL). The resulting solution was stirred at 25° C. for 1 hr, and then concentrated in vacuo. The residue was diluted with warm water (100 mL). The resulting solution of TFA salt of the product was decanted from dark-brown oily residue, which was additionally rinsed with warm water (3*50 mL). The combined aqueous solution was filtered through a cotton pad to remove traces of oily impurities, then basified to pH 11-12 with 10% aqueous sodium hydroxide solution and extracted with dichloromethane (2*60 mL). The combined organic extracts were dried over sodium sulfate and concentrated in vacuo to afford 3-[rac-(3S)-3-methyl-2,3,4,5-tetrahydropyridin-6-yl]-5-(trifluoromethyl)pyridine (2.9 g, 11.97 mmol, 46.57% yield) as brown solid, which was directly used in the next step.

LCMS(ESI): [M+H]⁺ m/z: calcd 243.2; found 243.2; Rt=0.759 min.

### Step 3: The Synthesis of 3-[rac-(2R,5S)-5-Methyl-2-piperidyl]-5-(trifluoromethyl)pyridine

Sodium Borohydride (249.09 mg, 6.58 mmol, 231.92 μL) was added in one portion at −15° C. to a stirred solution of 3-[rac-(3S)-3-methyl-2,3,4,5-tetrahydropyridin-6-yl]-5-(trifluoromethyl)pyridine (2.9 g, 11.97 mmol) in methanol (30 mL). The reaction mixture was stirred at −15° C. for 0.5 hr, and then was allowed to warm to 25° C. over 0.5 hr, and then concentrated in vacuo. The residue was diluted with water (30 mL) and extracted with dichloromethane (2*40 mL). The combined organic extracts were dried over sodium sulphate and concentrated in vacuo to afford crude 3-[rac-(2R,5S)-5-methyl-2-piperidyl]-5-(trifluoromethyl)pyridine (2.6 g, 10.64 mmol, 88.92% yield) as red gum, which was used directly in the next step.

LCMS(ESI): [M+H]⁺ m/z: calcd 245.2; found 245.2; Rt=0.492 min.

### Step 4: The Synthesis of 2,2,2-Trifluoroethyl 2-oxo-2-[rac-(2R,5S)-5-methyl-2-[5-(trifluoromethyl)-3-pyridyl]-1-piperidyl]acetate

2,2,2-Trifluoroethyl 2-chloro-2-oxo-acetate (496.04 mg, 2.60 mmol) was added dropwise to a stirred solution of 3-[rac-(2R,5S)-5-methyl-2-piperidyl]-5-(trifluoromethyl)pyridine (0.53 g, 2.17 mmol) and TEA (439.14 mg, 4.34 mmol, 604.87 μL) in THF (20 mL) at 0° C., stirred for 1 hr at 0° C. Reaction mixture was used in the next step.

LCMS(ESI): [M+H]⁺ m/z: calcd 397.2; found 397.2; Rt=1.317 min.

### Step 5: The Synthesis of 2-Oxo-2-[rac-(2R,5S)-5-methyl-2-[5-(trifluoromethyl)-3-pyridyl]-1-piperidyl]acetamide

Ammonia (735.43 mg, 43.18 mmol) was bubbled trough a reaction mixture at 0° C., stirred for 1 hr at 0° C. Reaction mixture was filtered, solid washed with THF (2×5 mL), filtrate evaporated in vacuo to give 2-oxo-2-[rac-(2R,5S)-5-methyl-2-[5-(trifluoromethyl)-3-pyridyl]-1-piperidyl]acetamide (0.66 g, 2.09 mmol, 96.95% yield).

LCMS(ESI): [M+H]⁺ m/z: calcd 316.2; found 316.2; Rt=1.133 min.

### Step 6: The Synthesis of N-[4-Amino-1-(2-trimethylsilylethoxymethyl)pyrazolo[4,3-c]pyridin-7-yl]-2-oxo-2-[rac-(2R,5S)-5-methyl-2-[5-(trifluoromethyl)-3-pyridyl]-1-piperidyl]acetamide

Copper (3.02 mg, 47.58 μmol), Copper (I) iodide (90.61 mg, 475.75 μmol, 16.12 μL), caesium carbonate (620.04 mg, 1.90 mmol) was added to a stirred solution of 2-oxo-2-[rac-(2R,5S)-5-methyl-2-[5-(trifluoromethyl)-3-pyridyl]-1-piperidyl]acetamide (0.3 g, 951.50 μmol), 7-bromo-1-(2-trimethylsilylethoxymethyl)pyrazolo[4,3-c]pyridin-4-amine (359.31 mg, 1.05 mmol), rac-(1R,2R)—N1,N2-dimethylcyclohexane-1,2-diamine (67.67 mg, 475.75 μmol) in 1,4-dioxane (7 mL) under Ar atmosphere and stirred at 110° C. for 48 hr in closed vial. Reaction mixture was filtered, solid washed with dioxane (2×3 mL), filtrate concentrated to give crude N-[4-amino-1-(2-trimethylsilylethoxymethyl)pyrazolo[4,3-c]pyridin-7-yl]-2-oxo-2-[rac-(2R,5S)-5-methyl-2-[5-(trifluoromethyl)-3-pyridyl]-1-piperidyl]acetamide (0.5 g, 865.54 μmol, 90.97% yield).

LCMS(ESI): [M+H]⁺ m/z: calcd 578.2; found 578.2; Rt=1.013 min.

### Step 7: The Synthesis of N-(4-Amino-1H-pyrazolo[4,3-c]pyridin-7-yl)-2-oxo-2-[rac-(2R,5S)-5-methyl-2-[5-(trifluoromethyl)-3-pyridyl]-1-piperidyl]acetamide (Compound 260)

Hydrogen chloride solution 4.0M in dioxane (3.16 g, 86.55 mmol, 3.94 mL) was added to a solution of N-[4-amino-1-(2-trimethylsilylethoxymethyl)pyrazolo[4,3-c]pyridin-7-yl]-2-oxo-2-[rac-(2R,5S)-5-methyl-2-[5-(trifluoromethyl)-3-pyridyl]-1-piperidyl]acetamide (0.5 g, 865.54 μmol) in Methanol (4 mL) and stirred at 20° C. for 18 hr. Volatiles was evaporated in vacuo, the residue triturred with IPA (5 mL), filtered, washed with IPA (5 mL), and submitted to HPLC (column: Chromatorex 18 SMB100-5T 100×19 mm 5 um; 0-40% 0-5 min H₂O/ACN/0.1% FA, flow rate: 30 mL/min) to give N-(4-amino-1H-pyrazolo[4,3-c]pyridin-7-yl)-2-oxo-2-[rac-(2R,5S)-5-methyl-2-[5-(trifluoromethyl)-3-pyridyl]-1-5 piperidyl]acetamide (24 mg, 48.64 μmol, 5.62% yield, HCOOH).

¹H NMR (600 MHz, dmso) δ 0.90-1.15 (m, 3H), 1.28-1.42 (m, 1H), 1.59-1.73 (m, 1H), 1.88-2.00 (m, 1H), 1.99-2.15 (m, 1H), 2.18-2.30 (m, 1H), 2.73-2.98 (m, 1H), 3.76-4.15 (m, 1H), 5.15-5.73 (m, 1H), 6.57-6.87 (m, 2H), 7.54-7.75 (m, 1H), 7.86-8.26 (m, 3H), 8.71-8.96 (m, 2H), 9.64-10.64 (m, 1H), 12.57-13.40 (m, 1H).

LCMS(ESI): [M+H]⁺ m/z: calcd 448.2; found 448.2; Rt=1.158 min.

### Example 231. N-(4-Amino-1H-pyrazolo[4,3-c]pyridin-7-yl)-2-oxo-2-[rac-(2R,5S)-2-(5-methoxy-3-pyridyl)-5-methyl-1-piperidyl]acetamide (Compound 261)

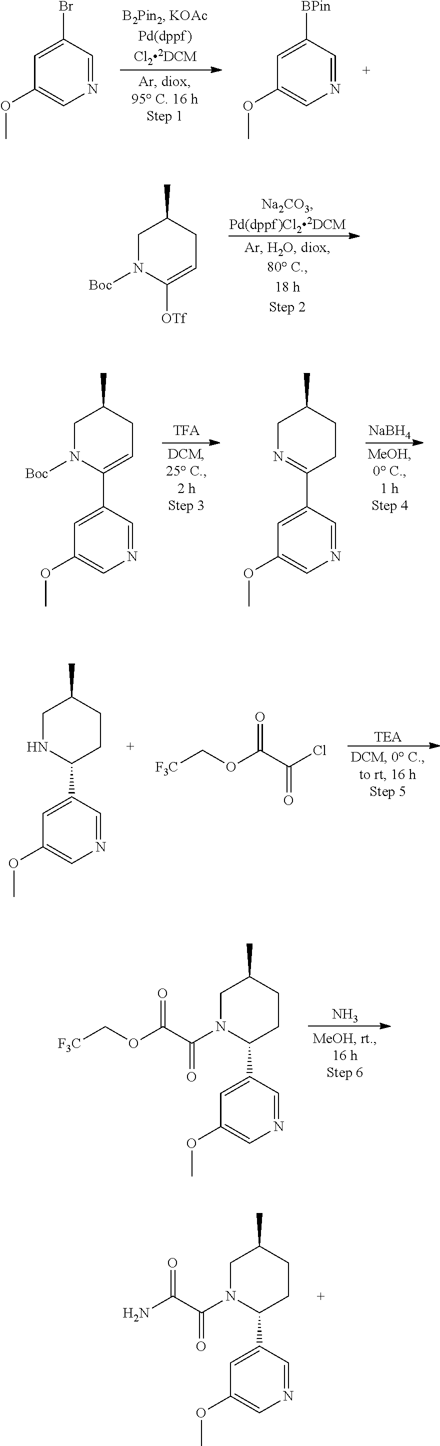

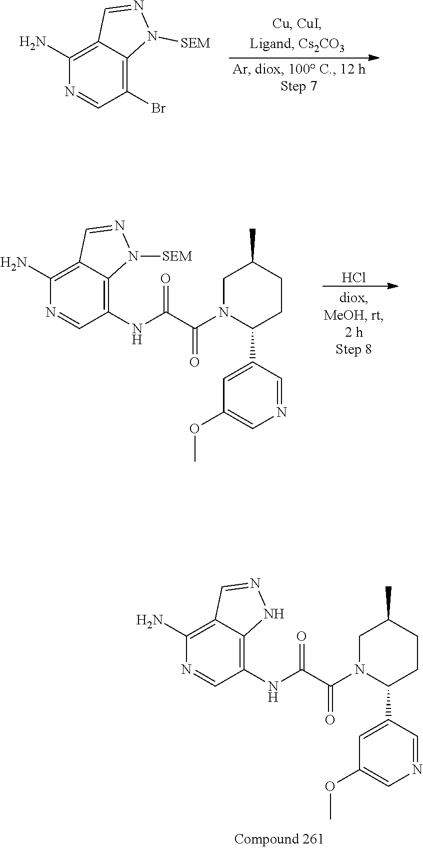 [SMILES: C[Si](C)(C)CCOCn1ncc2c(N)ncc(Br)c21 ; COc1cncc([C@H]2CC[C@H](C)CN2C(=O)C(=O)Nc2cnc(N)c3cnn(COCC[Si](C)(C)C)c23)c1 ; Cl ; COc1cncc([C@H]2CC[C@H](C)CN2C(=O)C(=O)Nc2cnc(N)c3cn[nH]c23)c1]

### Step 1: The Synthesis of 3-Methoxy-5-(4,4,5,5-tetramethyl-1,3,2-dioxaborolan-2-yl)pyridine

A mixture of 3-bromo-5-methoxy-pyridine (5 g, 26.59 mmol), 4,4,5,5-tetramethyl-2-(4,4,5,5-tetramethyl-1,3,2-dioxaborolan-2-yl)-1,3,2-dioxaborolane (7.43 g, 29.25 mmol) and Potassium Acetate (7.83 g, 79.78 mmol, 4.99 mL) in 1,4-dioxane (5.24 mL) was evacuated and then backfilled with argon. This operation was repeated two times, then Pd(dppf)Cl2·CH2Cl2 (2.17 g, 2.66 mmol) was added and the reaction mixture was stirred under argon at 95° C. for 16 hr. Then cooled down and concentrated in vacuo. The residue was diluted with MTBE (60 mL) and stirred for 0.5 hr. After most of the residue had dissolved, anhydrous sodium sulfate was added, and the resulting mixture was filtered. The filtrate was concentrated in vacuo to give 3-methoxy-5-(4,4,5,5-tetramethyl-1,3,2-dioxaborolan-2-yl)pyridine (4.3 g, 18.29 mmol, 68.78% yield).

### Step 2: The Synthesis of tert-Butyl rac-(3S)-6-(5-methoxy-3-pyridyl)-3-methyl-3,4-dihydro-2H-pyridine-1-carboxylate

tert-Butyl (3S)-3-methyl-6-(trifluoromethylsulfonyloxy)-3,4-dihydro-2H-pyridine-1-carboxylate (1.76 g, 5.10 mmol), Sodium carbonate (1.35 g, 12.76 mmol, 534.19 μL) and water (40.01 mL) were added to a solution of 3-methoxy-5-(4,4,5,5-tetramethyl-1,3,2-dioxaborolan-2-yl)pyridine (1 g, 4.25 mmol) in 1,4-dioxane (160.02 mL). The resulting mixture was evacuated and then backfilled with argon. This operation was repeated two times, then Pd(dppf)Cl₂·DCM (173.69 mg, 212.69 μmol) was added and the reaction mixture was stirred under argon at 80° C. for 18 hr, then cooled down and concentrated in vacuo. The residue was diluted with MTBE (60 mL) and stirred for 0.5 hr. After the main part of the residue dissolved, anhydrous sodium sulfate was added, and the resulting mixture was filtered. The filtercake was additionally washed with MTBE (3*20 mL) and discarded. The filtrate was concentrated in vacuo to afford crude tert-butyl rac-(3S)-6-(5-methoxy-3-pyridyl)-3-methyl-3,4-dihydro-2H-pyridine-1-carboxylate (1.2 g, 3.94 mmol, 92.68% yield) as brown gum, which was used directly in the next step.

LCMS(ESI): [M+H]⁺ m/z: calcd 305.2; found 305.2; Rt=1.152 min.

### Step 3: The Synthesis of 3-Methoxy-5-[rac-(3S)-3-methyl-2,3,4,5-tetrahydropyridin-6-yl]pyridine

Trifluoroacetic acid (6.74 g, 59.14 mmol, 4.56 mL) was added in one portion to a stirred solution of tert-butyl rac-(3S)-6-(5-methoxy-3-pyridyl)-3-methyl-3,4-dihydro-2H-pyridine-1-carboxylate (1.2 g, 3.94 mmol) in dichloromethane. The resulting solution was stirred at 25° C. for 0.5 hr, and then concentrated in vacuo. The residue was diluted with water (30 mL). The resulting solution of TFA salt of the product was decanted from dark-brown oily residue, which was additionally rinsed with water (2*10 mL). The combined aqueous solution was basified to pH 11-12 with 10% aqueous sodium carbonate solution and extracted with dichloromethane (2*20 mL). The combined organic extracts were dried over sodium sulfate and concentrated in vacuo to afford 3-methoxy-5-[rac-(3S)-3-methyl-2,3,4,5-tetrahydropyridin-6-yl]pyridine (0.8 g, 3.92 mmol, 99.34% yield) as brown gum, which was directly used in the next step.

LCMS(ESI): [M+H]⁺ m/z: calcd 205.2; found 205.2; Rt=0.542 min.

### Step 4: The Synthesis of 3-Methoxy-5-[rac-(2R,5S)-5-methyl-2-piperidyl]pyridine

Sodium Borohydride (740.79 mg, 19.58 mmol, 689.75 μL) was added in one portion at 0° C. to a stirred solution of 3-methoxy-5-[rac-(3S)-3-methyl-2,3,4,5-tetrahydropyridin-6-yl]pyridine (0.8 g, 3.92 mmol) in methanol (14.75 mL). The reaction mixture was stirred at 0° C. for 1 hr, and then concentrated in vacuo. The residue was diluted with water (10 mL) and extracted with dichloromethane (2*15 mL). The combined organic extracts were dried over sodium sulfate and concentrated in vacuo to afford crude 3-methoxy-5-[rac-(2R,5S)-5-methyl-2-piperidyl]pyridine (0.78 g, 3.78 mmol, 96.55% yield).

LCMS(ESI): [M+H]⁺ m/z: calcd 207.2; found 207.2; Rt=0.661 min.

### Step 5: The Synthesis of 2,2,2-Trifluoroethyl 2-oxo-2-[rac-(2R,5S)-2-(5-methoxy-3-pyridyl)-5-methyl-1-piperidyl]acetate

3-Methoxy-5-[rac-(2S,5R)-5-methyl-2-piperidyl]pyridine (0.78 g, 3.40 mmol) and TEA (344.36 mg, 3.40 mmol, 474.32 μL) was dissolved in DCM (40.49 mL), cooled with ice-water bath and then 2,2,2-trifluoroethyl 2-chloro-2-oxo-acetate (680.72 mg, 3.57 mmol) in 20 mL of DCM was added dropwise in 10 min. Solution was stirred overnight. Reaction mixture was washed with aqueous solution of NaHCO₃, dried over sodium sulphate and evaporated to give 2,2,2-trifluoroethyl 2-oxo-2-[rac-(2R,5S)-2-(5-methoxy-3-pyridyl)-5-methyl-1-piperidyl]acetate (0.85 g, 2.36 mmol, 69.32% yield).

LCMS(ESI): [M+H]⁺ m/z: calcd 361.2; found 361.2; Rt=1.208 min.

### Step 6: The Synthesis of 2-Oxo-2-[rac-(2R,5S)-2-(5-methoxy-3-pyridyl)-5-methyl-1-piperidyl]acetamide

2,2,2-Trifluoroethyl 2-oxo-2-[rac-(2R,5S)-2-(5-methoxy-3-pyridyl)-5-methyl-1-piperidyl]acetate (0.85 g, 2.36 mmol) was dissolved in NH₃/methanol (30 mL) solution (10% by weight) and stirred overnight, then concentrated in vacuo to give 2-oxo-2-[rac-(2R,5S)-2-(5-methoxy-3-pyridyl)-5-methyl-1-piperidyl]acetamide (0.6 g, 2.16 mmol, 91.72% yield).

LCMS(ESI): [M+H]⁺ m/z: calcd 278.2; found 278.2; Rt=0.686 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-2-(5-methoxy-3-pyridyl)-5-methyl-1-piperidyl]acetamide

7-Bromo-1-(2-trimethylsilylethoxymethyl)pyrazolo[4,3-c]pyridin-4-amine (185.69 mg, 540.89 μmol), 2-oxo-2-[rac-(2R,5S)-2-(5-methoxy-3-pyridyl)-5-methyl-1-piperidyl]acetamide (0.15 g, 540.89 μmol), Copper (I) iodide (30.90 mg, 162.27 μmol, 5.50 μL), Cesium carbonate (352.47 mg, 1.08 mmol) and (1R,2R)—N1,N2-dimethylcyclohexane-1,2-diamine (34.62 mg, 243.40 μmol) were mixed in Dioxane (3 mL) under argon, and then stirred overnight at 100° C. or 12 hr in vial. Reaction mixture was filtered, then evaporated. Reaction mixture was filtered, then evaporated. Crude product was dissolved in 2 mL of DMSO and subjected to HPLC to give Crude product was dissolved in 2 mL of DMSO and subjected to HPLC to give N-[4-amino-1-(2-trimethylsilylethoxymethyl)pyrazolo[4,3-c]pyridin-7-yl]-2-oxo-2-[rac-(2R,5S)-2-(5-methoxy-3-pyridyl)-5-methyl-1-piperidyl]acetamide (0.025 g, 46.32 μmol, 8.56% yield) Chromatography data:

2-10 min 30-100% water-methanol+fa; flow 30 mL/min.

LCMS(ESI): [M+H]⁺ m/z: calcd 538.2; found 538.2; Rt=1.163 min.

### Step 8: The Synthesis of N-(4-Amino-1H-pyrazolo[4,3-c]pyridin-7-yl)-2-oxo-2-[rac-(2R,5S)-2-(5-methoxy-3-pyridyl)-5-methyl-1-piperidyl]acetamide (Compound 261)

N-[4-Amino-1-(2-trimethylsilylethoxymethyl)pyrazolo[4,3-c]pyridin-7-yl]-2-oxo-2-[rac-(2R,5S)-2-(5-methoxy-3-pyridyl)-5-methyl-1-piperidyl]acetamide (25.00 mg, 46.32 μmol) was dissolved in MeOH (1 mL) and diox/HCl (926.44 μmol, 1 mL) was added thereto. Then it was stirred at rt for 2 hr. The reaction mixture was evaporated to afford N-(4-amino-1H-pyrazolo[4,3-c]pyridin-7-yl)-2-oxo-2-[rac-(2R,5S)-2-(5-methoxy-3-pyridyl)-5-methyl-1-piperidyl]acetamide (0.09 g, 219.81 μmol, 474.53% yield)

### Chromatography Data:

2-10 min 25-50% water-methanol+NH₃; flow 30 mL/min.

¹H NMR (600 MHz, dmso) δ 0.76-1.08 (m, 3H), 1.20-1.47 (m, 1H), 1.58-1.76 (m, 1H), 1.82-2.01 (m, 1H), 2.02-2.16 (m, 1H), 2.16-2.30 (m, 1H), 2.74-3.19 (m, 1H), 3.73-3.83 (m, 2H), 3.83-4.13 (m, 2H), 5.49-5.77 (m, 1H), 6.57-6.83 (m, 2H), 7.07-7.35 (m, 1H), 7.52-7.77 (m, 1H), 8.07-8.20 (m, 2H), 8.22-9.27 (m, 1H), 9.55-10.64 (m, 1H), 12.56-13.40 (m, 1H).

LCMS(ESI): [M+H]⁺ m/z: calcd 410.2; found 410.2; Rt=0.727 min.

### Example 232. N-(4-Amino-1H-pyrazolo[4,3-c]pyridin-7-yl)-2-oxo-2-[rac-(2R,5S)-2-(2-ethylindazol-5-yl)-5-methyl-1-piperidyl]acetamide (Compound 262)

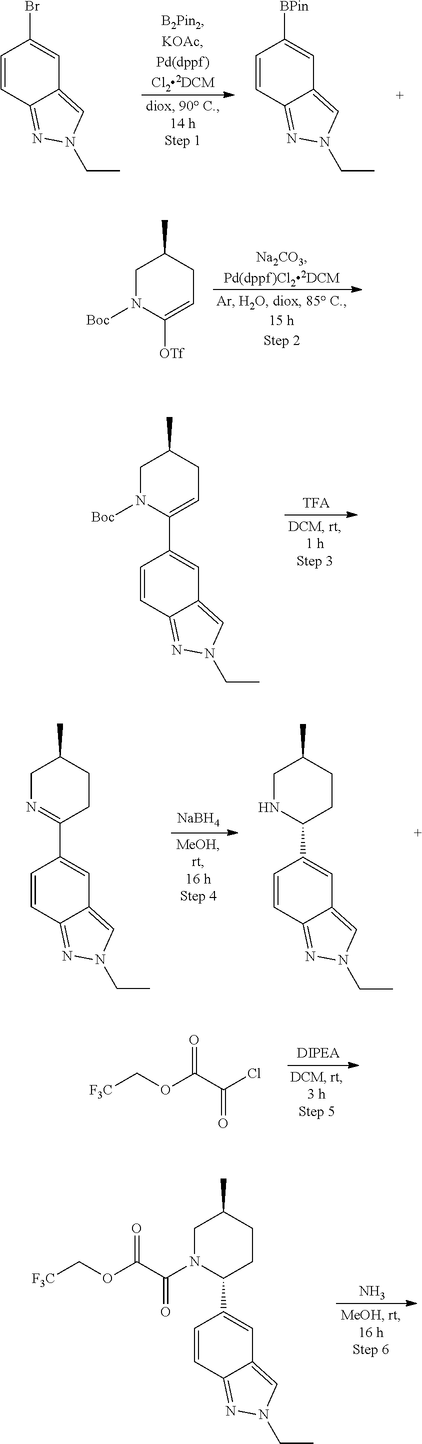

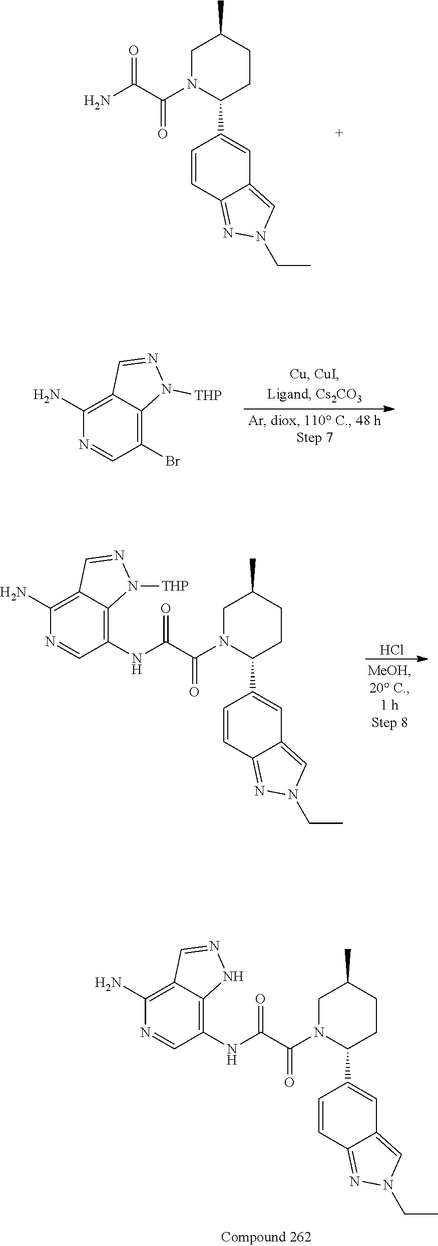

### Step 1: The Synthesis of 2-Ethyl-5-(4,4,5,5-tetramethyl-1,3,2-dioxaborolan-2-yl)indazole

A mixture of 5-bromo-2-ethyl-indazole (7 g, 31.10 mmol), 4,4,5,5-tetramethyl-2-(4,4,5,5-tetramethyl-1,3,2-dioxaborolan-2-yl)-1,3,2-dioxaborolane (8.69 g, 34.21 mmol), Pd(dppf)Cl₂·DCM (1.27 g, 1.55 mmol) and Potassium Acetate (9.16 g, 93.30 mmol, 5.83 mL) was stirred in 1,4-dioxane (150 mL) at 90° C. for 14 hr. Upon completion, the reaction mixture was diluted with water and extracted with DCM (3×100 mL). Combined organic phases were dried over Na₂SO₄, filtered through a pad of SiO₂ and concentrated under reduced pressure to afford 2-ethyl-5-(4,4,5,5-tetramethyl-1,3,2-dioxaborolan-2-yl)indazole (7.1 g, crude).

LCMS(ESI): [M+H]⁺ m/z: calcd 273.2; found 273.2; Rt=1.285 min.

### Step 2: The Synthesis of tert-Butyl rac-(3S)-6-(2-ethylindazol-5-yl)-3-methyl-3,4-dihydro-2H-pyridine-1-carboxylate

2-Ethyl-5-(4,4,5,5-tetramethyl-1,3,2-dioxaborolan-2-yl)indazole (7.1 g, 26.09 mmol), tert-butyl rac-(3S)-3-methyl-6-(trifluoromethylsulfonyloxy)-3,4-dihydro-2H-pyridine-1-carboxylate (15.32 g, 44.35 mmol), Sodium carbonate (8.30 g, 78.27 mmol, 3.28 mL) and Pd(dppf)Cl2 DCM (1.07 g, 1.30 mmol) were stirred in a mixture of 1,4-dioxane (75 mL) and water (25 mL) under inert atmosphere at 85° C. for 15 hr. Upon completion, the reaction mixture was cooled down, diluted with water and extracted with DCM. The organic layer was separated, dried over Na₂SO₄ and concentrated under reduced pressure to afford tert-butyl rac-(3S)-6-(2-ethylindazol-5-yl)-3-methyl-3,4-dihydro-2H-pyridine-1-carboxylate (8.5 g, crude) which was used in the next step without purification.

LCMS(ESI): [M+H]⁺ m/z: calcd 342.2; found 342.2; Rt=1.164 min.

### Step 3: The Synthesis of 2-Ethyl-5-[rac-(3S)-3-methyl-2,3,4,5-tetrahydropyridin-6-yl]indazole

tert-Butyl rac-(3S)-6-(2-ethylindazol-5-yl)-3-methyl-3,4-dihydro-2H-pyridine-1-carboxylate (7.5 g, 21.97 mmol) was dissolved in a mixture of TFA (2.96 g, 25.96 mmol, 2 mL) and DCM (3 mL) and stirred at RT for 0.5 hr. Upon completion, reaction mixture was concentrated under reduced pressure and the residue was dissolved in DCM, washed with NaHCO₃ solution, dried over Na₂SO₄ and concentrated under reduced pressure to afford 2-ethyl-5-[rac-(3S)-3-methyl-2,3,4,5-tetrahydropyridin-6-yl]indazole (4.8 g, crude).

LCMS(ESI): [M+H]⁺ m/z: calcd 242.2; found 242.2; Rt=0.794 min.

### Step 4: The Synthesis of 2-Ethyl-5-[rac-(2R,5S)-5-methyl-2-piperidyl]indazole

Sodium Borohydride (1.50 g, 39.78 mmol, 1.40 mL) was added portionwise to a stirred solution of 2-ethyl-5-[rac-(3S)-3-methyl-2,3,4,5-tetrahydropyridin-6-yl]indazole (4.8 g, 19.89 mmol) in methanol (50 mL). Reaction mixture was stirred at RT overnight and concentrated. The residue was dissolved in DCM, washed with NaHCO₃ solution, dried over Na₂SO₄ and concentrated under reduced pressure to afford 2-ethyl-5-[rac-(2R,5S)-5-methyl-2-piperidyl]indazole (4 g, crude).

LCMS(ESI): [M+H]⁺ m/z: calcd 244.2; found 244.2; Rt=0.592 min.

### Step 5: The Synthesis of Ethyl 2-oxo-2-[rac-(2R,5S)-2-(2-ethylindazol-5-yl)-5-methyl-1-piperidyl]acetate

Ethyl 2-chloro-2-oxo-acetate (2.36 g, 17.26 mmol, 1.93 mL) was added dropwise to an ice bath cooled stirred solution of 2-ethyl-5-[rac-(2R,5S)-5-methyl-2-piperidyl]indazole (4 g, 16.44 mmol) and DIPEA (2.76 g, 21.37 mmol, 3.72 mL) in DCM. Upon completion, the reaction mixture was washed with water. The organic layer was separated, dried over Na₂SO₄ and concentrated under reduced pressure. The residue was purified by flash column chromatography to afford ethyl 2-oxo-2-[rac-(2R,5S)-2-(2-ethylindazol-5-yl)-5-methyl-1-piperidyl]acetate (2 g, 5.82 mmol, 35.43% yield).

### Chromatography:

Interchim 120 g SiO₂, HEX-MTBE from 0˜100%, flow rate=70 mL/min, cv=24.2

LCMS(ESI): [M+H]⁺ m/z: calcd 344.2; found 344.2; Rt=1.345 min.

### Step 6: The Synthesis of 2-Oxo-2-[rac-(2R,5S)-2-(2-ethylindazol-5-yl)-5-methyl-1-piperidyl]acetamide

Ethyl 2-oxo-2-[rac-(2R,5S)-2-(2-ethylindazol-5-yl)-5-methyl-1-piperidyl]acetate (2 g, 5.82 mmol) was dissolved in saturated ammonia solution in methanol. The reaction mixture was stirred overnight and concentrated under reduced pressure to afford 2-oxo-2-[rac-(2R,5S)-2-(2-ethylindazol-5-yl)-5-methyl-1-piperidyl]acetamide (1.6 g, crude).

LCMS(ESI): [M+H]⁺ m/z: calcd 315.2; found 315.2; Rt=0.988 min.

### Step 7: The Synthesis of N-(4-Amino-1-tetrahydropyran-2-yl-pyrazolo[4,3-c]pyridin-7-yl)-2-oxo-2-[rac-(2R,5S)-2-(2-ethylindazol-5-yl)-5-methyl-1-piperidyl]acetamide

Copper (6.06 mg, 95.43 μmol), Copper (I) iodide (181.74 mg, 954.26 μmol, 32.34 μL), caesium carbonate (1.24 g, 3.82 mmol) was added to a stirred solution of 2-oxo-2-[rac-(2R,5S)-2-(2-ethylindazol-5-yl)-5-methyl-1-piperidyl]acetamide (0.6 g, 1.91 mmol), 7-bromo-1-tetrahydropyran-2-yl-pyrazolo[4,3-c]pyridin-4-amine (595.47 mg, 2.00 mmol), rac-(1R,2R)—N1,N2-dimethylcyclohexane-1,2-diamine (135.73 mg, 954.26 μmol) in 1,4-dioxane (20.00 mL) under Ar atmosphere and stirred at 110° C. for 48 hr in closed vial. Reaction mixture was filtered, solid washed with dioxane (2×3 mL), filtrate concentrated and purified by flash chromatography to give crude starting oxamide.

LCMS(ESI): [M+H]⁺ m/z: calcd 531.2; found 531.2; Rt=1.070 min.

### Step 8: The Synthesis of N-(4-Amino-1H-pyrazolo[4,3-c]pyridin-7-yl)-2-oxo-2-[rac-(2R,5S)-2-(2-ethylindazol-5-yl)-5-methyl-1-piperidyl]acetamide (Compound 262)

Hydrogen chloride solution 4.0M in dioxane (1.72 g, 47.11 mmol, 2.15 mL) was added to a solution of N-(4-amino-1-tetrahydropyran-2-yl-pyrazolo[4,3-c]pyridin-7-yl)-2-oxo-2-[rac-(2R,5S)-2-(2-ethylindazol-5-yl)-5-methyl-1-piperidyl]acetamide (0.25 g, 471.15 μmol) in Methanol (1 mL) and stirred at 20° C. for 1 hr. Volatiles was evaporated in vacuo, the residue triturred with IPA (5 mL), filtered, washed with IPA (5 mL), and submitted to HPLC (column: Chromatorex 18 SMB100-5T 100×19 mm 5 um; 5-25% 0-5 min H₂O/ACN/0.1% FA, flow rate: 30 mL/min) to give N-(4-amino-1H-pyrazolo[4,3-c]pyridin-7-yl)-2-oxo-2-[rac-(2R,5S)-2-(2-ethylindazol-5-yl)-5-methyl-1-piperidyl]acetamide (50 mg, 103.53 μmol, 21.97% yield, HCl).

¹H NMR (500 MHz, dmso) δ 0.06-1.09 (m, 3H), 1.23-1.43 (m, 1H), 1.45-1.52 (m, 3H), 1.53-2.34 (m, 4H), 2.63-2.83 (m, 1H), 3.43-4.16 (m, 1H), 4.34-4.50 (m, 2H), 4.98-5.79 (m, 1H), 5.99-7.35 (m, 3H), 7.51-8.41 (m, 5H), 9.54-10.63 (m, 1H), 11.20-14.20 (m, 2H).

LCMS(ESI): [M+2H]⁺ m/z: calcd 448.2; found 448.2; Rt=2.324 min.

### Example 233. N-(4-Amino-2H-pyrazolo[4,3-c]pyridin-7-yl)-2-oxo-2-[rac-(2R,5S)-2-[4-[2-(dimethylamino)ethyl]phenyl]-5-methyl-1-piperidyl]acetamide (Compound 263)

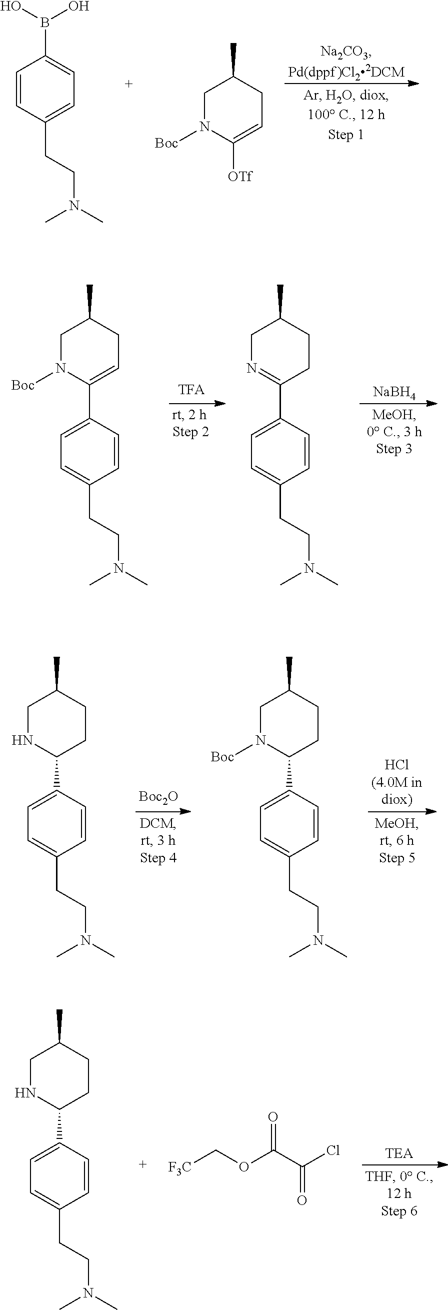

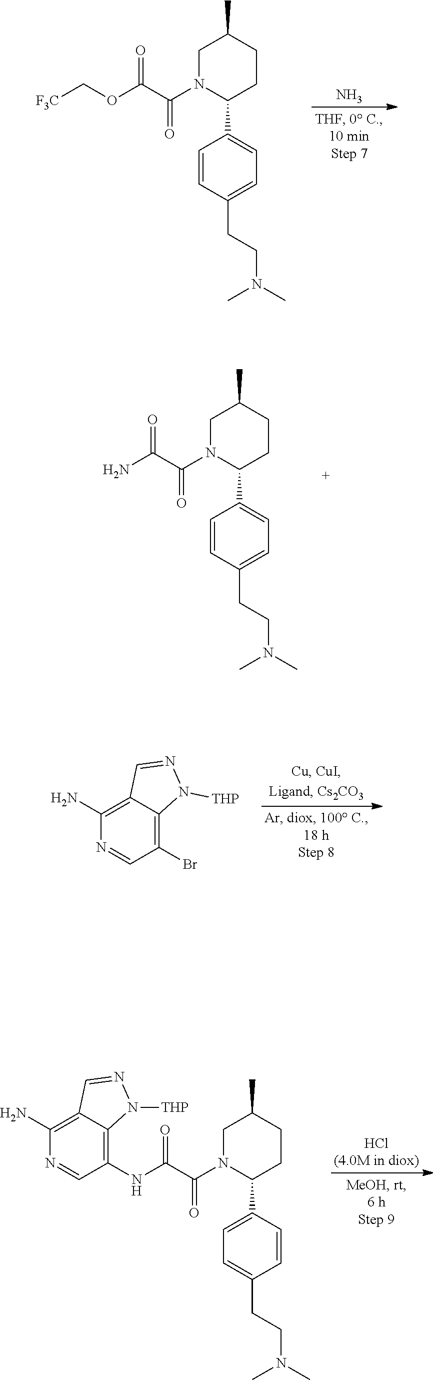

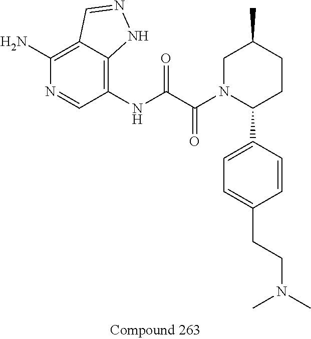 [SMILES: C[C@H]1CC[C@H](c2ccc(CCN(C)C)cc2)N(C(=O)C(=O)Nc2cnc(N)c3cn[nH]c23)C1]

### Step 1: The Synthesis of tert-Butyl rac-(3S)-6-[4-[2-(dimethylamino)ethyl]phenyl]-3-methyl-3,4-dihydro-2H-pyridine-1-carboxylate

To a solution of [4-[2-(dimethylamino)ethyl]phenyl]boronic acid (5.2 g, 22.66 mmol, HCl), tert-butyl rac-(3S)-3-methyl-6-(trifluoromethylsulfonyloxy)-3,4-dihydro-2H-pyridine-1-carboxylate (9.39 g, 27.19 mmol) and Sodium carbonate (9.61 g, 90.63 mmol, 3.79 mL) in Water (40.15 mL) and Dioxane (120.44 mL), Pd(dppf)Cl₂*CH₂Cl₂ (925.12 mg, 1.13 mmol) was added under argon atmosphere. The resulting mixture was stirred at 100° C. for 12 hr, cooled, filtered and evaporated. The residue was diluted with MTBE (150 mL), dried over Na₂SO₄, filtered and evaporated in vacuo to obtain tert-butyl rac-(3S)-6-[4-[2-(dimethylamino)ethyl]phenyl]-3-methyl-3,4-dihydro-2H-pyridine-1-carboxylate (10 g, crude).

LCMS(ESI): [M+H]⁺ m/z: calcd 345.2; found 345.2; Rt=0.925 min.

### Step 2: The Synthesis of N,N-Dimethyl-2-[4-[rac-(3S)-3-methyl-2,3,4,5-tetrahydropyridin-6-yl]phenyl]ethanamine

A solution of tert-butyl rac-(3S)-6-[4-[2-(dimethylamino)ethyl]phenyl]-3-methyl-3,4-dihydro-2H-pyridine-1-carboxylate (7.8 g, 22.64 mmol) in Trifluoroacetic acid (35 g, 306.96 mmol, 23.65 mL) was stirred at 25° C. for 2 hr. The residue was diluted with water (50 mL). pH of the aqueous layer was adjusted to 9-10 with NaOH solution, extracted with DCM (3*30 mL), dried over Na₂SO₄ and evaporated to give N,N-dimethyl-2-[4-[rac-(3S)-3-methyl-2,3,4,5-tetrahydropyridin-6-yl]phenyl]ethanamine (4.5 g, 18.41 mmol, 81.33% yield).

LCMS(ESI): [M+H]⁺ m/z: calcd 245.2; found 245.2; Rt=0.249 min.

### Step 3: The Synthesis of N,N-Dimethyl-2-[4-[rac-(2R,5S)-5-methyl-2-piperidyl]phenyl]ethanamine

Sodium Borohydride (696.66 mg, 18.41 mmol, 648.66 μL) was added portionwise to a stirred solution of N,N-dimethyl-2-[4-[rac-(3S)-3-methyl-2,3,4,5-tetrahydropyridin-6-yl]phenyl]ethanamine (4.5 g, 18.41 mmol) in Methanol (60 mL) at 0° C. The resulting mixture was stirred at 0° C. for 3 hr, and then evaporated in vacuo. The residue was diluted with water (100 mL) and extracted with dichloromethane (3*80 mL). The combined organic extracts were dried over sodium sulphate and evaporated in vacuo to afford N,N-dimethyl-2-[4-[rac-(2R,5S)-5-methyl-2-piperidyl]phenyl]ethanamine (4.5 g, 18.26 mmol, 99.18% yield).

LCMS(ESI): [M+H]⁺ m/z: calcd 247.2; found 247.2; Rt=0.644 min.

### Step 4: The Synthesis of tert-Butyl rac-(2R,5S)-2-[4-[2-(dimethylamino)ethyl]phenyl]-5-methyl-piperidine-1-carboxylate

A solution of N,N-dimethyl-2-[4-[rac-(2R,5S)-5-methyl-2-piperidyl]phenyl]ethanamine (4.5 g, 18.26 mmol) and Di-tert-butyl dicarbonate (3.99 g, 18.26 mmol, 4.19 mL) in DCM (40 mL) was stirred at 25° C. for 3 hr and evaporated. The residue was purified by gradient chromatography (ACN-MeOH) to obtain tert-butyl rac-(2R,5S)-2-[4-[2-(dimethylamino)ethyl]phenyl]-5-methyl-piperidine-1-carboxylate (2.5 g, 7.21 mmol, 39.50% yield).

### Step 5: The Synthesis of N,N-Dimethyl-2-[4-[rac-(2R,5S)-5-methyl-2-piperidyl]phenyl]ethanamine

A solution of tert-butyl rac-(2R,5S)-2-[4-[2-(dimethylamino)ethyl]phenyl]-5-methyl-piperidine-1-carboxylate (2.5 g, 7.21 mmol) in MeOH (49.67 mL) and Hydrogen chloride solution 4.0M in dioxane (263.06 mg, 7.21 mmol, 328.83 L) was stirred at 25° C. for 6 hr and evaporated in vacuo to give N,N-dimethyl-2-[4-[rac-(2R,5S)-5-methyl-2-piperidyl]phenyl]ethanamine (2.25 g, 7.05 mmol, 97.66% yield, 2HCl).

LCMS(ESI): [M+H]⁺ m/z: calcd 247.2; found 247.2; Rt=0.593 min.

### Step 6: The Synthesis of 2,2,2-Trifluoroethyl 2-oxo-2-[rac-(2R,5S)-2-[4-[2-(dimethylamino)ethyl]phenyl]-5-methyl-1-piperidyl]acetate

To a solution of N,N-dimethyl-2-[4-[rac-(2R,5S)-5-methyl-2-piperidyl]phenyl]ethanamine (1.35 g, 4.23 mmol, 2HCl) and Triethylamine (3.42 g, 33.82 mmol, 4.71 mL) in THF (38.78 mL), 2,2,2-trifluoroethyl 2-chloro-2-oxo-acetate (1.61 g, 8.46 mmol) was added portionwise at 0° C. The resulting mixture was stirred for 12 hr. LCMS showed full conversion of SM. The reaction mixture was directly used for the next step.

LCMS(ESI): [M+H]⁺ m/z: calcd 401.2; found 401.2; Rt=0.984 min.

### Step 7: The Synthesis of 2-Oxo-2-[rac-(2R,5S)-2-[4-[2-(dimethylamino)ethyl]phenyl]-5-methyl-1-piperidyl]acetamide

Through a solution of 2,2,2-trifluoroethyl 2-oxo-2-[rac-(2R,5S)-2-[4-[2-(dimethylamino)ethyl]phenyl]-5-methyl-1-piperidyl]acetate (1.65 g, 4.12 mmol) in THF (50 mL), ammonia was bubbled during 10 min at 0° C. The formed precipitate was filtered off, washed with THF (30 mL) and the solvent was evaporated in vacuo to give crude product (3 g), which was purified by gradient chromatography (MTBE-MeOH) 2-oxo-2-[rac-(2R,5S)-2-[4-[2-(dimethylamino)ethyl]phenyl]-5-methyl-1-piperidyl]acetamide (0.95 g, 2.99 mmol, 72.63% yield).

LCMS(ESI): [M+H]⁺ m/z: calcd 318.2; found 318.2; Rt=0.573 min.

### Step 8: The Synthesis of N-(4-Amino-2-tetrahydropyran-2-yl-pyrazolo[4,3-c]pyridin-7-yl)-2-oxo-2-[rac-(2R,5S)-2-[4-[2-(dimethylamino)ethyl]phenyl]-5-methyl-1-piperidyl]acetamide

Two portions. For each portion 300 mg, vial (8 mL) was used.

To a mixture of 2-oxo-2-[rac-(2R,5S)-2-[4-[2-(dimethylamino)ethyl]phenyl]-5-methyl-1-piperidyl]acetamide (0.3 g, 945.11 μmol), 7-bromo-2-tetrahydropyran-2-yl-pyrazolo[4,3-c]pyridin-4-amine (421.26 mg, 1.42 mmol), Copper (12.01 mg, 189.02 μmol), Copper (I) iodide (180.00 mg, 945.11 μmol, 32.03 μL), Cesium carbonate (615.87 mg, 1.89 mmol) and rac-(1R,2R)—N1,N2-dimethylcyclohexane-1,2-diamine (201.65 mg, 1.42 mmol), Dioxane (5 mL) was added. The resulting mixture was evacuated, refiled with Argon three time, heated at 100° C. for 18 hr and cooled. This compound was combined with another batch. The inorganic precipitate was filtered off and washed with DCM (30 mL) to give N-(4-amino-2-tetrahydropyran-2-yl-pyrazolo[4,3-c]pyridin-7-yl)-2-oxo-2-[rac-(2R,5S)-2-[4-[2-(dimethylamino)ethyl]phenyl]-5-methyl-1-piperidyl]acetamide (0.5 g, 936.92 μmol, 99.13% yield).

This substance was used for the next step without further purification.

LCMS(ESI): [M+H]⁺ m/z: calcd 534.2; found 534.4; Rt=0.697 min.

### Step 9: The Synthesis of N-(4-Amino-2H-pyrazolo[4,3-c]pyridin-7-yl)-2-oxo-2-[rac-(2R,5S)-2-[4-[2-(dimethylamino)ethyl]phenyl]-5-methyl-1-piperidyl]acetamide (Compound 263)

To a solution of N-(4-amino-2-tetrahydropyran-2-yl-pyrazolo[4,3-c]pyridin-7-yl)-2-oxo-2-[rac-(2R,5S)-2-[4-[2-(dimethylamino)ethyl]phenyl]-5-methyl-1-piperidyl]acetamide (0.5 g, 936.92 μmol) in MeOH (10 mL), Hydrogen chloride, 4M in 1,4-dioxane, 99% (4 g, 109.71 mmol, 5.00 mL) was added. The resulting mixture was stirred at 25° C. for 6 hr and evaporated in vacuo. The residue was purified by HPLC (Device (Mobile Phase, Column): SYSTEM 5-5-25% 0-1-5 min H₂O/ACN/0.1% FA, flow: 30 mL/min (loading pump 4 mL/min H₂O); Chromatorex 18 SMB100-5T 100×19 mm 5 um) and re-purified (Device (Mobile Phase, Column): SYSTEM 30-80% 0-5 min H₂O/MeOH/0.1% NH₄OH, flow: 30 mL/min (loading pump 4 mL/min methanol); column: XBridge C18 100×19 mm, 5 um) to obtain desired product (201 mg). This substance was re-purified by chiral chromatography (Column: Chiralcel OJ-H (250*20 mm, 5 mkm); Mobile phase: Hexane-MeOH-IPA-DEA, 60-20-20-0.1 Flow Rate: 12 mL/min; 19 injections, V=13.5 l, 20 h) to obtain N-(4-amino-2H-pyrazolo[4,3-c]pyridin-7-yl)-2-oxo-2-[rac-(2R,5S)-2-[4-[2-(dimethylamino)ethyl]phenyl]-5-methyl-1-piperidyl]acetamide (157 mg, 349.24 μmol, 37.28% yield).

¹H NMR (600 MHz, dmso) δ 0.78-1.06 (m, 3H), 1.21-1.39 (m, 1H), 1.62-1.76 (m, 1H), 1.79-2.08 (m, 2H), 2.10-2.26 (m, 7H), 2.38-2.45 (m, 2H), 2.63-3.25 (m, 3H), 3.40-4.09 (m, 1H), 4.85-5.64 (m, 1H), 6.18-7.18 (m, 3H), 7.19-7.52 (m, 3H), 7.53-7.76 (m, 1H), 8.10-8.24 (m, 1H), 9.46-10.61 (m, 1H), 12.46-13.44 (m, 1H).

LCMS(ESI): [M+2H]⁺ m/z: calcd 451.2; found 451.2; Rt=1.879 min.

### Example 234. N-(4-Amino-1H-pyrazolo[4,3-c]pyridin-7-yl)-2-oxo-2-[rac-(2R,5S)-2-(3-chloro-7-quinolyl)-5-methyl-1-piperidyl]acetamide (Compound 264)

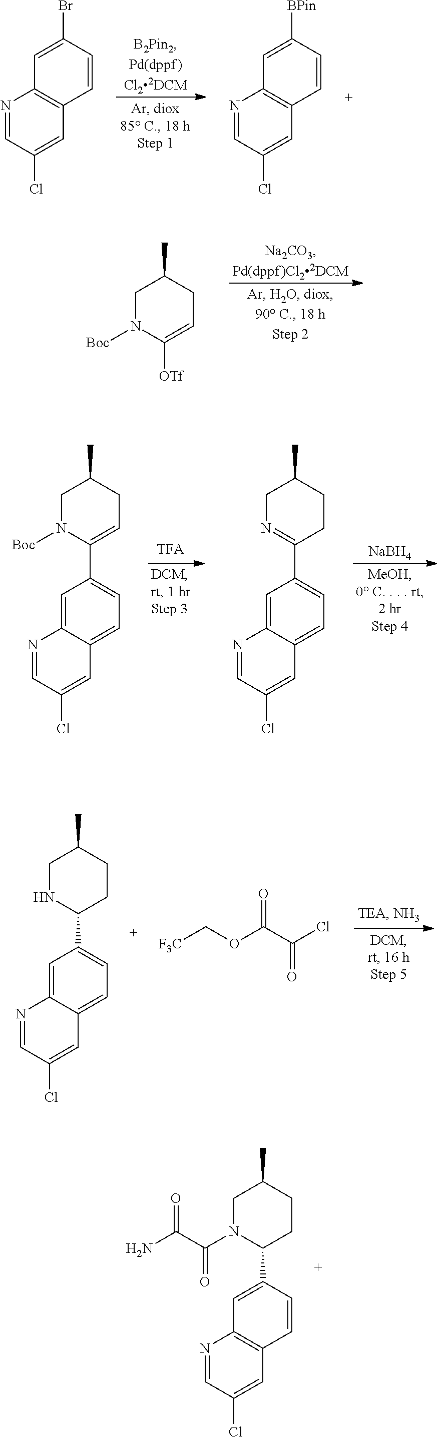

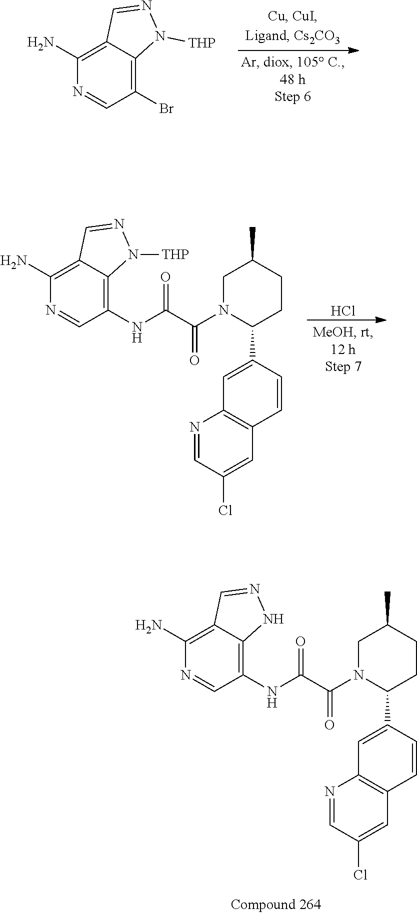

### Step 1: The Synthesis of 3-Chloro-7-(4,4,5,5-tetramethyl-1,3,2-dioxaborolan-2-yl)quinoline

A mixture of 7-bromo-3-chloro-quinoline (3 g, 12.37 mmol), 4,4,5,5-tetramethyl-2-(4,4,5,5-tetramethyl-1,3,2-dioxaborolan-2-yl)-1,3,2-dioxaborolane (3.46 g, 13.61 mmol) and Potassium Acetate (3.04 g, 30.93 mmol, 1.93 mL) in 1,4-dioxane (100 mL) was evacuated and then backfilled with argon. This operation was repeated two times, then Pd(dppf)Cl2·CH2Cl2 (1.01 g, 1.24 mmol) was added and the reaction mixture was stirred under argon at 85° C. for 18 hr. The LCMS of the aliquot showed complete conversion of starting bromide. The reaction mixture contained crude 3-chloro-7-(4,4,5,5-tetramethyl-1,3,2-dioxaborolan-2-yl)quinoline (3.58 g, crude) (yield based on 1H NMR and LCMS) was used directly in the next step.

LCMS(ESI): [M+H]⁺ m/z: calcd 290.2; found 290.2; Rt=1.633 min.

### Step 2: The Synthesis of tert-Butyl rac-(3S)-6-(3-chloro-7-quinolyl)-3-methyl-3,4-dihydro-2H-pyridine-1-carboxylate

tert-Butyl rac-(3S)-3-methyl-6-(trifluoromethylsulfonyloxy)-3,4-dihydro-2H-pyridine-1-carboxylate (4.70 g, 13.60 mmol), Sodium carbonate (3.93 g, 37.09 mmol, 1.55 mL) and water (100 mL) were added to a solution of crude from previous step 3-chloro-7-(4,4,5,5-tetramethyl-1,3,2-dioxaborolan-2-yl)quinoline (3.58 g, 12.36 mmol) in 1,4-dioxane (18 mL). The resulting mixture was evacuated and then backfilled with argon, this operation was repeated three times, then Pd(dppf)Cl2DCM (504.82 mg, 618.17 μmol) was added under argon. The reaction mixture was stirred under argon at 90° C. for 18 hr. The reaction mixture was cooled and then filtered. The filtercake was additionally washed with 1,4-dioxane (2*25 mL) and discarded. The combined filtrate was concentrated in vacuo to afford crude tert-butyl rac-(3S)-6-(3-chloro-7-quinolyl)-3-methyl-3,4-dihydro-2H-pyridine-1-carboxylate (13 g, crude) as black gum, which was used directly in the next step.

LCMS(ESI): [M+H]⁺ m/z: calcd 359.2; found 359.2; Rt=1.552 min.

### Step 3: The Synthesis of 3-Chloro-7-[rac-(3S)-3-methyl-2,3,4,5-tetrahydropyridin-6-yl]quinoline

Trifluoroacetic acid (59.20 g, 519.21 mmol, 40 mL) was added in one portion to a stirred solution of tert-butyl rac-(3S)-6-(3-chloro-7-quinolyl)-3-methyl-3,4-dihydro-2H-pyridine-1-carboxylate (13 g, 36.23 mmol) in dichloromethane (40 mL). The resulting solution was stirred at 25° C. for 1 hr, and then concentrated in vacuo. The residue was diluted with warm water (200 mL). The resulting solution of TFA salt of the product was decanted from dark-brown oily residue, which was additionally rinsed with warm water (3*50 mL). The combined aqueous solution was filtered through a cotton pad to remove traces of oily impurities, then basified to pH 11-12 with 10% aqueous sodium hydroxide solution and extracted with dichloromethane (2*70 mL). The combined organic extracts were dried over sodium sulfate and concentrated in vacuo to afford 3-chloro-7-[rac-(3S)-3-methyl-2,3,4,5-tetrahydropyridin-6-yl]quinoline (1.4 g, 5.41 mmol, 14.94% yield) as beige solid, which was used directly in the next step.

LCMS(ESI): [M+H]⁺ m/z: calcd 259.0; found 259.0; Rt=0.894 min.

### Step 4: The Synthesis of 3-Chloro-7-[rac-(2R,5S)-5-methyl-2-piperidyl]quinoline

Sodium Borohydride (220 mg, 5.82 mmol, 204.84 μL was added in one portion at 0° C. to a stirred solution of 3-chloro-7-[rac-(3S)-3-methyl-2,3,4,5- tetrahydropyridin-6-yl]quinoline (1.4 g, 5.41 mmol) in methanol (30 mL). The reaction mixture was stirred at 0° C. for 1 hr, and then was allowed to warm to 25° C. over 0.5 hr, and then concentrated in vacuo. The residue was diluted with water (30 mL) and extracted with dichloromethane (2*40 mL). The combined organic extracts were dried over sodium sulphate and concentrated in vacuo to afford crude 3-chloro-7-[rac-(2R,5S)-5-methyl-2-piperidyl]quinoline (1.1 g, 4.22 mmol, 77.96% yield) as beige solid, which was used directly in the next step.

LCMS(ESI): [M+H]⁺ m/z: calcd 261.2; found 261.2; Rt=0.798 min.

### Step 5: The Synthesis of 2-Oxo-2-[rac-(2R,5S)-2-(3-chloro-7-quinolyl)-5-methyl-1-piperidyl]acetamide

To a solution of 3-chloro-7-[rac-(2R,5S)-5-methyl-2-piperidyl]quinoline (1.1 g, 4.22 mmol) and Triethylamine (853.72 mg, 8.44 mmol, 1.18 mL) in THF (20.02 mL), 2,2,2-trifluoroethyl 2-chloro-2-oxo-acetate (1.21 g, 6.33 mmol) was added portionwise at 25° C. The resulting mixture was stirred for 2 hr. LCMS showed full conversion of SM. Through resulting mixture Ammonia (71.84 mg, 4.22 mmol) was bubbled during 20 min at 25° C. The formed precipitate was filtered off, washed with THF (30 mL) and the solvent was evaporated in vacuo to give 2-oxo-2-[rac-(2R,5S)-2-(3-chloro-7-quinolyl)-5-methyl-1-piperidyl]acetamide (1.0 g, crude).

### Step 6: The Synthesis of N-(4-Amino-2-tetrahydropyran-2-yl-pyrazolo[4,3-c]pyridin-7-yl)-2-oxo-2-[rac-(2R,5S)-2-(3-chloro-7-quinolyl)-5-methyl-1-piperidyl]acetamide

A mixture of 2-oxo-2-[rac-(2R,5S)-2-(3-chloro-7-quinolyl)-5-methyl-1-piperidyl]acetamide (200 mg, 602.78 μmol), 7-bromo-2-tetrahydropyran-2-yl-pyrazolo[4,3-c]pyridin-4-amine (268.67 mg, 904.17 μmol), copper (5 mg, 78.68 μmol), Copper (I) iodide (130 mg, 682.59 μmol, 23.13 μL), caesium carbonate (294.60 mg, 904.17 μmol) and rac-(1R,2R)—N1,N2-dimethylcyclohexane-1,2-diamine (130 mg, 913.95 μmol) in 1,4-dioxane (6.00 mL) was stirred in a sealed vial under argon at 105° C. for 48 hr. The resulting mixture was cooled down and filtered. The filtercake was washed successively with THF (2*5 mL) and dichloromethane (3*5 mL). The combined filtrate was concentrated in vacuo to afford N-(4-amino-2-tetrahydropyran-2-yl-pyrazolo[4,3-c]pyridin-7-yl)-2-oxo-2-[rac-(2R,5S)-2-(3-chloro-7-quinolyl)-5-methyl-1-piperidyl]acetamide (620 mg, crude) as brown gum, which was used directly in the next step.

LCMS(ESI): [M+H]⁺ m/z: calcd 548.2; found 548.2; Rt=3.263 min.

### Step 7: The Synthesis of N-(4-Amino-1H-pyrazolo[4,3-c]pyridin-7-yl)-2-oxo-2-[rac-(2R,5S)-2-(3-chloro-7-quinolyl)-5-methyl-1-piperidyl]acetamide (Compound 264)

Hydrogen chloride solution 4.0M in dioxane (4.20 g, 16.01 mmol, 4.00 mL, 13.9% purity) was added to a stirred solution of crude from previous step N-(4-amino-2-tetrahydropyran-2-yl-pyrazolo[4,3-c]pyridin-7-yl)-2-oxo-2-[rac-(2R,5S)-2-(3-chloro-7-quinolyl)-5-methyl-1-piperidyl]acetamide (675.15 mg, 1.23 mmol) in methanol (4 mL) at 25° C. The resulting solution was stirred at 25° C. for 12 hr, then concentrated to dryness in vacuo and the residue was submitted to reverse phase HPLC (column: Chromatorex 18 SMB100-5T 100×19 mm 5 um; mobile phase: 10-10-50% 0-1-5 min H₂O/Acetonitrile/0.2% FA; flow: 30 mL/min (loading pump 4 mL/min acetonitrile)) to afford Compound 264 N-(4-amino-1H-pyrazolo[4,3-c]pyridin-7-yl)-2-oxo-2-[rac-(2R,5S)-2-(3-chloro-7-quinolyl)-5-methyl-1-piperidyl]acetamide (101 mg, 198.06 μmol, 16.08% yield, HCOOH) as beige solid.

¹H NMR (500 MHz, dmso) δ 0.92-1.15 (m, 3H), 1.27-1.48 (m, 1H), 1.66-1.79 (m, 1H), 1.85-1.98 (m, 1H), 2.06-2.30 (m, 1H), 2.37-2.43 (m, 1H), 2.76-3.18 (m, 1H), 3.67-4.18 (m, 1H), 4.98-6.05 (m, 1H), 6.93-7.56 (m, 2H), 7.55-7.80 (m, 2H), 7.88-8.06 (m, 2H), 8.16-8.40 (m, 1H), 8.51-8.61 (m, 1H), 8.82-8.93 (m, 1H), 9.57-10.91 (m, 1H), 13.03 (s, 1H).

LCMS(ESI): [M+H]⁺ m/z: calcd 464.4; found 464.4; Rt=1.741 min.

### Example 235. N-(4-Amino-1H-pyrazolo[4,3-c]pyridin-7-yl)-2-oxo-2-[rac-(2R,5S)-5-methyl-2-(2-methyl-3,4-dihydro-1H-isoquinolin-6-yl)-1-piperidyl]acetamide (Compound 265)

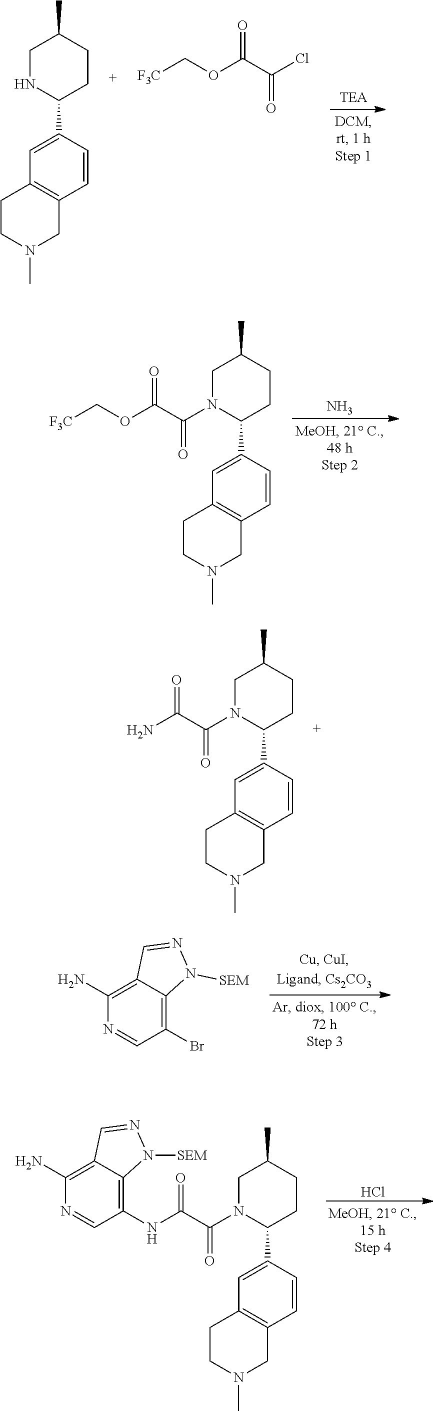

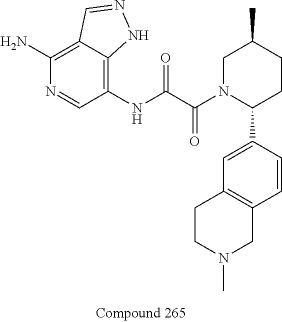 [SMILES: C[C@H]1CC[C@H](c2ccc3c(c2)CCN(C)C3)N(C(=O)C(=O)Nc2cnc(N)c3cn[nH]c23)C1]

### Step 1: The Synthesis of 2,2,2-Trifluoroethyl 2-oxo-2-[rac-(2R,5S)-5-methyl-2-(2-methyl-3,4-dihydro-1H-isoquinolin-6-yl)-1-piperidyl]acetate

To a solution of 2-methyl-6-[rac-(2R,5S)-5-methyl-2-piperidyl]-3,4-dihydro-1H-isoquinoline (0.6 g, 2.46 mmol) and TEA (248.45 mg, 2.46 mmol, 342.21 μL) in DCM (25 mL) was added 2,2,2-trifluoroethyl 2-chloro-2-oxo-acetate (467.74 mg, 2.46 mmol) at rt. After stirring at rt for 1 hr the resulting mixture was washed with water, dried and evaporated to dryness to give 2,2,2-trifluoroethyl 2-oxo-2-[rac-(2R,5S)-5-methyl-2-(2-methyl-3,4-dihydro-1H-isoquinolin-6-yl)-1-piperidyl]acetate (0.9 g, 2.26 mmol, 92.00% yield) as a yellow gum and was used in the next step without further purification.

LCMS(ESI): [M+H]⁺ m/z: calcd 399.2; found 399.2; Rt=2.712 min.

### Step 2: The Synthesis of 2-Oxo-2-[rac-(2R,5S)-5-methyl-2-(2-methyl-3,4-dihydro-1H-isoquinolin-6-yl)-1-piperidyl]acetamide

Ammonia (38.47 mg, 2.26 mmol) was bubbled through a solution of 2,2,2-trifluoroethyl 2-oxo-2-[rac-(2R,5S)-5-methyl-2-(2-methyl-3,4-dihydro-1H-isoquinolin-6-yl)-1-piperidyl]acetate (0.9 g, 2.26 mmol) in MeOH (25 mL) at rt. After stirring for 18 hr, the reaction mixture was evaporated to dryness to give 2-oxo-2-[rac-(2R,5S)-5-methyl-2-(2-methyl-3,4-dihydro-1H-isoquinolin-6-yl)-1-piperidyl]acetamide (0.7 g, 2.22 mmol, 98.25% yield) as a yellow solid.

LCMS(ESI): [M+H]⁺ m/z: calcd 316.2; found 316.2; Rt=1.750 min.

### Step 3: The Synthesis of N-[4-Amino-1-(2-trimethylsilylethoxymethyl)pyrazolo[4,3-c]pyridin-7-yl]-2-oxo-2-[rac-(2R,5S)-5-methyl-2-(2-methyl-3,4-dihydro-1H-isoquinolin-6-yl)-1-piperidyl]acetamide

2-Oxo-2-[rac-(2R,5S)-5-methyl-2-(2-methyl-3,4-dihydro-1H-isoquinolin-6-yl)-1-piperidyl]acetamide (150 mg, 475.57 μmol), 7-bromo-1-(2-trimethylsilylethoxymethyl)pyrazolo[4,3-c]pyridin-4-amine (163.26 mg, 475.57 μmol), Copper (I) iodide (45.29 mg, 237.79 μmol, 8.06 μL), Cesium carbonate (309.90 mg, 951.15 μmol) and (1R,2R)—N1,N2-dimethylcyclohexane-1,2-diamine (67.65 mg, 475.57 μmol) were mixed in dioxane (5.00 mL) under argon, and then stirred for 72 h at 100° C. for 72 hr in vial. The reaction mixture was filtered and the filtrate was concentrated in vacuo and the residue was purified by HPLC.

### Step 4: The Synthesis of N-(4-Amino-1H-pyrazolo[4,3-c]pyridin-7-yl)-2-oxo-2-[rac-(2R,5S)-5-methyl-2-(2-methyl-3,4-dihydro-1H-isoquinolin-6-yl)-1-piperidyl]acetamide (Compound 265)

To a solution of N-[4-amino-1-(2-trimethylsilylethoxymethyl)pyrazolo[4,3-c]pyridin-7-yl]-2-oxo-2-[rac-(2R,5S)-5-methyl-2-(2-methyl-3,4-dihydro-1H-isoquinolin-6-yl)-1-piperidyl]acetamide (18.7 mg, 32.36 μmol) in MeOH (0.5 mL) was added Hydrogen chloride solution 4.0M in dioxane (400.00 mg, 10.97 mmol, 0.5 mL) at 21° C. The resulting mixture was left to stir for 15 hr. The resulting mixture was evaporated to dryness and subjected to HPLC (1st run—30-55% 2-7 min; 30 mL/min water-MeCN+NH₃ (loading pump 4 mL/min MeCN); column xbridge C18 19*100 mm (L); 2nd run—0-25% 2-8 min; 30 mL/min water-MeCN+FA (loading pump 4 mL/min MeCN); column SunfireC18 19*100 mm (R)). N-(4-amino-1H-pyrazolo[4,3-c]pyridin-7-yl)-2-oxo-2-[rac-(2R,5S)-5-methyl-2-(2-methyl-3,4-dihydro-1H-isoquinolin-6-yl)-1-piperidyl]acetamide (3.7 mg, 7.50 μmol, 23.16% yield, HCOOH) was obtained as a yellow solid.

LCMS(ESI): [M+H]⁺ m/z: calcd 448.2; found 448.2; Rt=1.446 min.

### Example 236. N-(4-amino-1H-pyrazolo[4,3-c]pyridin-7-yl)-2-((2S,5S)-5-methyl-2-(phenyl-d5)piperidin-1-yl)-2-oxoacetamide (Compound 286) and N-(4-amino-1H-pyrazolo[4,3-c]pyridin-7-yl)-2-((2R,5S)-5-methyl-2-(phenyl-d5)piperidin-1-yl)-2-oxoacetamide (Compound 268)

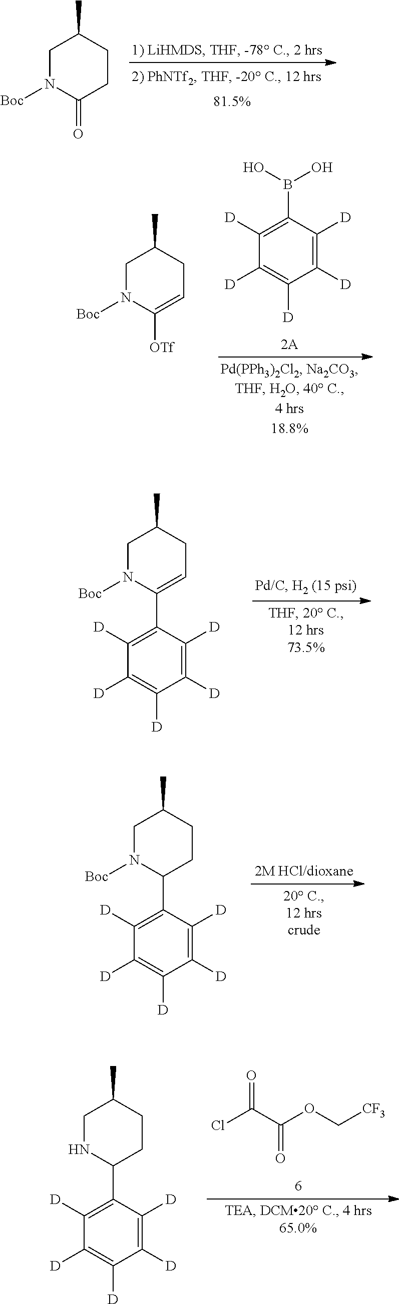

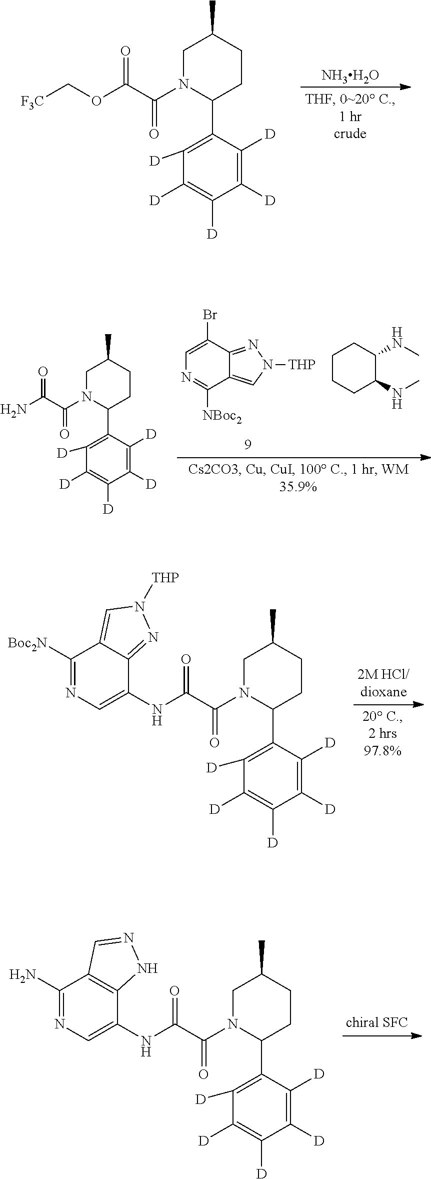

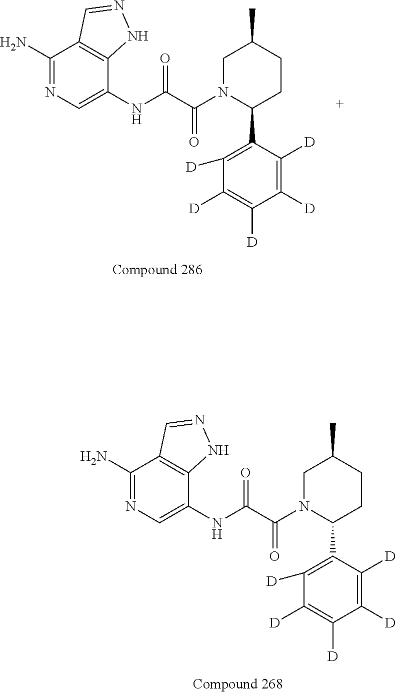 [SMILES: [2H]c1c([2H])c([2H])c([C@@H]2CC[C@H](C)CN2C(=O)C(=O)Nc2cnc(N)c3cn[nH]c23)c([2H])c1[2H] ; [2H]c1c([2H])c([2H])c([C@H]2CC[C@H](C)CN2C(=O)C(=O)Nc2cnc(N)c3cn[nH]c23)c([2H])c1[2H]]

### Step 1: Synthesis of tert-butyl (3S)-3-methyl-6-(trifluoromethylsulfonyloxy)-3,4-dihydro-2H-pyridine-1-carboxylate

To a solution of tert-butyl (5S)-5-methyl-2-oxo-piperidine-1-carboxylate (5 g, 23.4 mmol) in THF (50 mL) at 20° C. was added LiHMDS (1 M, 35.0 mL) at −78° C. and the mixture was stirred at −78° C.° C. for 2 hours, then PHNTf₂ (9.2 g, 25.75 mmol) was added to the mixture at −78° C. and the mixture was stirred at 20° C. for 12 hours. The reaction was quenched by addition of saturated NH₄Cl aqueous solution (100 mL), and then extracted with EtOAc (50 mL*3). The combined organic layers were dried over anhydrous Na₂SO₄, filtered and concentrated under reduced pressure. The residue was purified by flash chromatography (ISCOR; 80 g AgelaFlash® Silica Flash Column, petroleum ether/EtOAc with EtOAc from 0˜0%, flow rate=60 mL/min) to afford tert-butyl (3S)-3-methyl-6-(trifluoromethylsulfonyloxy)-3,4-dihydro-2H-pyridine-1-carboxylate (6.6 g, 81.5% yield) as yellow oil. ¹H NMR (400 MHZ, chloroform-d) 8 ppm 5.25 (t, J=3.76 Hz, 1H), 3.87 (dd, J=12.67, 3.39 Hz, 1H), 3.00 (dd, J=12.67, 9.16 Hz, 1H), 2.35-2.47 (m, 1H), 2.35-2.47 (m, 1H), 1.76-1.99 (m, 2H), 1.49 (s, 9H), 0.99 (d, J=6.53 Hz, 3H).

### Step 2: Synthesis of tert-butyl (3S)-3-methyl-6-(2,3,4,5,6-pentadeuteriophenyl)-3,4-dihydro-2H-pyridine-1-carboxylate

To a solution of tert-butyl (3S)-3-methyl-6-(trifluoromethylsulfonyloxy)-3,4-dihydro-2H-pyridine-1-carboxylate (6.6 g, 19.1 mmol) and (2,3,4,5,6-pentadeuteriophenyl) boronic acid (4.88 g, 38.6 mmol) in THF (60 mL) and H₂O (5 mL) was added Pd(PPh₃)Cl₂ (1.32 g, 1.88 mmol) at 20° C. The mixture was stirred at 40° C. for 4 hours. The reaction mixture was quenched by addition water (50 mL) at 20° C., extracted with EtOAc (50 mL*3). The combined organic layers were washed with brine (20 mL*3), dried over anhydrous Na₂SO₄, filtered and concentrated under reduced pressure. The residue was purified by flash chromatography (ISCOR; 80 g AgelaFlash® Silica Flash Column, petroleum ether/EtOAc with EtOAc from 0˜5%, flow rate=60 mL/min, 254 nm) to afford tert-butyl (3S)-3-methyl-6-(2,3,4,5,6-pentadeuteriophenyl)-3,4-dihydro-2H-pyridine-1-carboxylate (1 g, 18.8% yield) as colorless oil. ¹H NMR (400 MHZ, chloroform-d) δ ppm 5.30 (t, J=3.76 Hz, 1H), 2.99 (dd, J=12.30, 9.79 Hz, 1H), 2.40 (dd, J=18.57, 6.40, 3.89 Hz, 1H), 1.96-2.04 (m, 1H), 1.80-1.92 (m, 1H), 1.60-1.71 (m, 1H), 1.07 (s, 9H), 1.02 (d, J=6.53 Hz, 3H).

### Step 3: Synthesis of tert-butyl (5S)-5-methyl-2-(2,3,4,5,6-pentadeuteriophenyl)piperidine-1-carboxylate

To a solution of tert-butyl (3S)-3-methyl-6-(2,3,4,5,6-pentadeuteriophenyl)-3,4-dihydro-2H-pyridine-1-carboxylate (1 g, 3.59 mmol) in THF (20 mL) was added Pd/C (200 mg, 0.188 mmol, 10% purity) and the reaction mixture was stirred at 20° C. for 12 hours. Pd/C was filtrated by Celite. The filtrate was concentrated in under reduced pressure to afford tert-butyl (5S)-5-methyl-2-(2,3,4,5,6-pentadeuteriophenyl)piperidine-1-carboxylate (740 mg, 73.5% yield) as colorless oil. LCMS(ESI) [M+H]⁺ m/z: calcd 281.2., found 281.1.

### Step 4: Synthesis of ethyl (5S)-5-methyl-2-(2,3,4,5,6-pentadeuteriophenyl)piperidine

To a solution of tert-butyl (5S)-5-methyl-2-(2,3,4,5,6-pentadeuteriophenyl)piperidine-1-carboxylate (740 mg, 2.64 mmol) in dioxane (6.36 mL) was added HCl (2 M, 1.64 mL). The reaction mixture was stirred at 20° C. for 12 hours. The reaction mixture concentrated under reduced pressure to afford (5S)-5-methyl-2-(2,3,4,5,6-pentadeuteriophenyl)piperidine (680 mg, crude) as yellow oil. LCMS(ESI) [M+H]⁺ m/z: calcd 181.2, found 181.1.

### Step 5: Synthesis of 2,2,2-trifluoroethyl 2-oxo-2-[(5S)-5-methyl-2-(2,3,4,5,6-pentadeuteriophenyl)-1-piperidyl]acetate

To a mixture of (5S)-5-methyl-2-(2,3,4,5,6-pentadeuteriophenyl)piperidine (680 mg, 3.77 mmol), TEA (570 mg, 5.63 mmol) in DCM (8 mL) at 0° C. was added 2,2,2-trifluoroethyl 2-chloro-2-oxo-acetate (2.16 g, 11.3 mmol), the resulting mixture stirred at 20° C. for 12 hours. The reaction mixture was quenched by addition water (30 mL) at 20° C., extracted with DCM (30 mL*3). The combined organic layers were washed with brine (30 mL*3), dried over anhydrous Na₂SO₄, filtered and concentrated under reduced pressure. The residue was purified by flash silica gel chromatography (ISCOR; 20 g SepaFlash® Silica Flash Column, Petroleum ether/EtOAc with EtOAc from 0˜20%, Flow Rate: 35 mL/min, 254 nm) to afford 2,2,2-trifluoroethyl 2-oxo-2-[(5S)-5-methyl-2-(2,3,4,5,6-pentadeuteriophenyl)-1-piperidyl]acetate (820 mg, 65.0% yield) as yellow oil. LCMS(ESI)

[M+H]⁺ m/z: calcd 335.2, found 335.2.

### Step 6: Synthesis of 2-oxo-2-[(5S)-5-methyl-2-(2,3,4,5,6-pentadeuteriophenyl)-1-piperidyl]acetamide

To a mixture of 2,2,2-trifluoroethyl 2-oxo-2-[(5S)-5-methyl-2-(2,3,4,5,6-pentadeuteriophenyl)-1-piperidyl]acetate (820 mg, 2.45 mmol) in THF (5 mL) was added NH₃—H₂O (5 mL, 2.45 mmol) at 20° C. for 1 hour. The reaction mixture concentrated under reduced pressure to afford 2-oxo-2-[(5S)-5-methyl-2-(2,3,4,5,6-pentadeuteriophenyl)-1-piperidyl]acetamide (660 mg, crude) as colorless oil. LCMS(ESI) [M+H]⁺ m/z: calcd 252.2, found 252.1.

### Step 7: Synthesis of tert-butyl N-tert-butoxycarbonyl-N-[7-[[2-oxo-2-[(5S)-5-methyl-2-(2,3,4,5,6-pentadeuteriophenyl)-1-piperidyl]acetyl]amino]-2-tetrahydropyran-2-yl-pyrazolo[4,3-c]pyridin-4-yl]carbamate

To a mixture of 2-oxo-2-[(5S)-5-methyl-2-(2,3,4,5,6-pentadeuteriophenyl)-1-piperidyl]acetamide (660 mg, 2.63 mmol), tert-butyl N-(7-bromo-2-tetrahydropyran-2-yl-pyrazolo[4,3-c]pyridin-4-yl)-N-tert-butoxycarbonyl-carbamate (1.1 g, 2.21 mmol), rac-(1S,2S)—N1,N2-dimethylcyclohexane-1,2-diamine (440 mg, 3.09 mmol), Cs₂CO₃ (1.43 g, 4.39 mmol) in dioxane (13.0 mL) at 20° C. was added Cu (154 mg, 2.42 mmol) and CuI (440 mg, 2.31 mmol), the resulting mixture stirred at 100° C. for 1 hour under microwave irradiation. The reaction mixture was quenched by addition water (50 mL) at 20° C., extracted with EtOAc (50 mL*3). The combined organic layers were washed with brine (50 mL*3), dried over anhydrous Na₂SO₄, filtered and concentrated under reduced pressure. The residue was purified by flash silica gel chromatography (ISCOR; 20 g SepaFlash® Silica Flash Column, Petroleum ether/EtOAc with EtOAc from 0˜30%, Flow Rate: 35 mL/min, 254 nm) to afford tert-butyl N-tert-butoxycarbonyl-N-[7-[[2-oxo-2-[(5S)-5-methyl-2-(2,3,4,5,6-pentadeuteriophenyl)-1-piperidyl]acetyl]amino]-2-tetrahydropyran-2-yl-pyrazolo[4,3-c]pyridin-4-yl]carbamate (530 mg, 35.9% yield) as yellow oil. LCMS(ESI) [M+H]⁺ m/z: calcd 668.4, found 668.3.

### Step 8: Synthesis of N-(4-amino-1H-pyrazolo[4,3-c]pyridin-7-yl)-2-((5S)-5-methyl-2-(phenyl-d5)piperidin-1-yl)-2-oxoacetamide

To a solution of tert-butyl N-tert-butoxycarbonyl-N-[7-[[2-oxo-2-[(5S)-5-methyl-2-(2,3,4,5,6-pentadeuteriophenyl)-1-piperidyl]acetyl]amino]-2-tetrahydropyran-2-yl-pyrazolo[4,3-c]pyridin-4-yl]carbamate (320 mg, 0.479 mmol) in HCl/MeOH (2 M, 6 mL) and the reaction mixture was stirred at 20° C. for 12 hours. The mixture concentrated under reduced pressure. The mixture concentrated under reduced pressure. The reaction mixture was concentrated under reduced pressure. The residue was purified by preparative HPLC (Instrument: Gilson GX-281 Liquid Handler, Gilson 322 Pump, Gilson 156 UV Detector; Column: 2 Phenomenex Gemini C18 75*40 mm*3 um; Mobile phase A: H₂O with 10 mm NH₃—H₂O-AVN (v %); Mobile phase B: ACN; Gradient: B from 33% to 63% in 7.8 min, hold 100% B for 3 min; Flow Rate: 30 mL/min; Column Temperature: 30° C.; Wavelength: 220 nm, 254 nm) to afford N-(4-amino-1H-pyrazolo[4,3-c]pyridin-7-yl)-2-((5S)-5-methyl-2-(phenyl-d5)piperidin-1-yl)-2-oxoacetamide (180 mg, 97.8% yield) as a white solid. HPLC: 82.92% @220 nm, 84.19% @254 nm.

### Step 9: Synthesis of N-(4-amino-1H-pyrazolo[4,3-c]pyridin-7-yl)-2-((2S,5S)-5-methyl-2-(phenyl-d5)piperidin-1-yl)-2-oxoacetamide (Compound 286) and N-(4-amino-1H-pyrazolo[4,3-c]pyridin-7-yl)-2-((2R,5S)-5-methyl-2-(phenyl-d5)piperidin-1-yl)-2-oxoacetamide (Compound 268)

N-(4-amino-1H-pyrazolo[4,3-c]pyridin-7-yl)-2-((5S)-5-methyl-2-(phenyl-d5)piperidin-1-yl)-2-oxoacetamide was purified by chiral SFC (Instrument: Berger, MULTIGR AM-II; Column: DAICEL CHIRALPAK IC (250 mm*30 mm, 10 um; Mobile phase: supercritical CO₂/EtOH (0.1% NH₃—H₂O, v %)=35/35; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to afford Compound 286 and Compound 268.

Compound 286: N-(4-amino-1H-pyrazolo[4,3-c]pyridin-7-yl)-2-((2S,5S)-5-methyl-2-(phenyl-d5)piperidin-1-yl)-2-oxoacetamide (103 mg, 55.9% yield, peak 1, retention time=2.057 min, white solid). ¹H NMR (400 MHZ, methanol-d₄) δ ppm 8.20-8.29 (m, 1H), 7.52-7.75 (m, 1H), 5.45-5.94 (m, 1H), 3.93 (br d, J=9.79 Hz, 1H), 2.70-2.86 (m, 1H), 2.61 (br d, J=10.79 Hz, 1H), 2.27-2.44 (m, 1H), 1.86-2.12 (m, 1H), 1.78 (br d, J=12.05 Hz, 1H), 1.29 (br d, J=12.80 Hz, 1H), 0.82-0.92 (m, 3H); LCMS(ESI) [M+H]⁺ m/z: calcd 384.2, found 384.1; HPLC: 97.63% @220 nm, 100.00% @254 nm.

Compound 268: N-(4-amino-1H-pyrazolo[4,3-c]pyridin-7-yl)-2-((2R,5S)-5-methyl-2-(phenyl-d5)piperidin-1-yl)-2-oxoacetamide (8.6 mg, 4.67% yield, peak 2, retention time=2.261 min, white solid). ¹H NMR (400 MHZ, DMSO-d₆) δ ppm 12.41-13.27 (m, 1H), 9.22-10.50 (m, 1H), 8.20 (s, 1H), 7.37-7.93 (m, 1H), 6.34-6.82 (m, 2H), 4.91-5.75 (m, 1H), 3.71-4.15 (m, 1H), 2.19 (br s, 2H), 1.93 (br s, 1H), 1.76 (br s, 1H), 1.22-1.43 (m, 1H), 1.07 (br d, J=6.78 Hz, 3H); LCMS(ESI) [M+H]⁺ m/z: calcd 384.2, found 384.1; HPLC: 96.19% @220 nm, 95.98% @254 nm.

### Example 237. N-(4-Amino-1H-pyrazolo[4,3-c]pyridin-7-yl)-2-oxo-2-[rac-(2R,6R)-2-methyl-6-phenyl-1-piperidyl]acetamide (Compound 270)

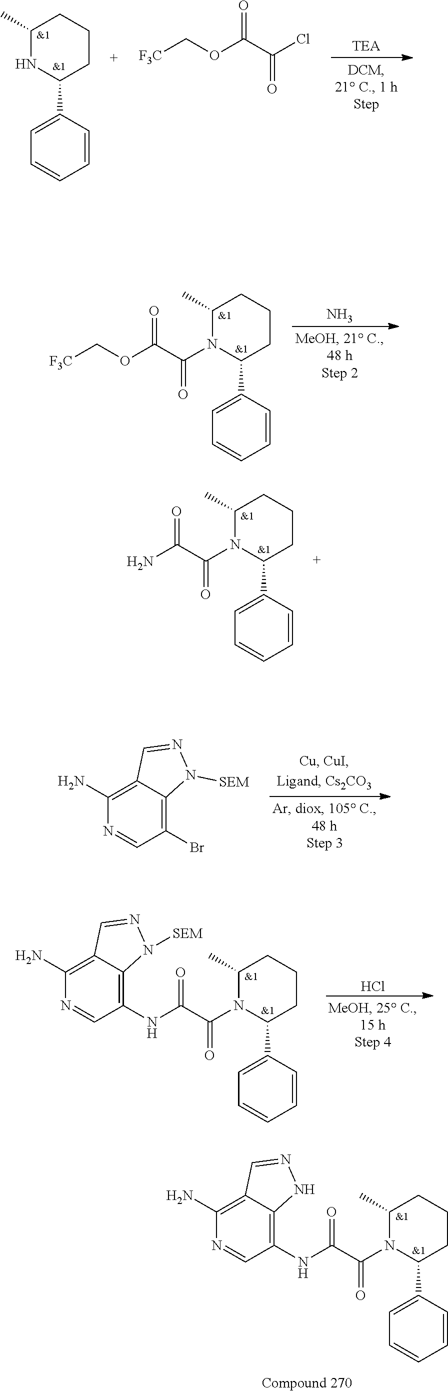

### Step 1: The Synthesis of 2,2,2-Trifluoroethyl 2-oxo-2-[rac-(2R,6R)-2-methyl-6-phenyl-1-piperidyl]acetate

To a solution of rac-(2S,6S)-2-methyl-6-phenyl-piperidine (0.97 g, 5.53 mmol) and TEA (672.02 mg, 6.64 mmol, 925.65 μL) in DCM (24.69 mL) was added 2,2,2-trifluoroethyl 2-chloro-2-oxo-acetate (1.05 g, 5.53 mmol) at rt. After stirring at rt for 1 hr the resulting mixture was washed with water, dried, and evaporated to dryness to give 2,2,2-trifluoroethyl 2-oxo-2-[rac-(2R,6R)-2-methyl-6-phenyl-1-piperidyl]acetate (1.7 g, 5.16 mmol, 93.28% yield) as an yellow gum solid and was used in the next step without further purification.

### Step 2: The Synthesis of 2-Oxo-2-[rac-(2R,6R)-2-methyl-6-phenyl-1-piperidyl]acetamide

Ammonium was bubbled through a solution of 2,2,2-trifluoroethyl 2-oxo-2-[rac-(2R,6R)-2-methyl-6-phenyl-1-piperidyl]acetate (1.8 g, 5.47 mmol) in MeOH (25 mL) at rt. After stirring for 18 hr, the reaction mixture was evaporated to dryness to give 2-oxo-2-[rac-(2R,6R)-2-methyl-6-phenyl-1-piperidyl]acetamide (1.6 g, crude) as a yellow gum.

LCMS(ESI): [M+H]⁺ m/z: calcd 247.2; found 247.2; Rt=2.506 min.

### Step 3: The Synthesis of N-[4-Amino-1-(2-trimethylsilylethoxymethyl)pyrazolo[4,3-c]pyridin-7-yl]-2-oxo-2-[rac-(2R,6R)-2-methyl-6-phenyl-1-piperidyl]acetamide

A mixture of 2-oxo-2-[rac-(2R,6R)-2-methyl-6-phenyl-1-piperidyl]acetamide (250 mg, 1.02 mmol), 7-bromo-1-(2-trimethylsilylethoxymethyl)pyrazolo[4,3-c]pyridin-4-amine (383.29 mg, 1.12 mmol), copper (3.83 mg, 60.30 μmol), Copper (I) iodide (110 mg, 577.58 μmol, 19.57 μL), caesium carbonate (496.06 mg, 1.52 mmol) and rac-(1R,2R)—N1,N2-dimethylcyclohexane-1,2-diamine (110 mg, 773.34 μmol) in 1,4-dioxane (6 mL) was stirred in a sealed vial under argon at 105° C. for 48 hr. The resulting mixture was cooled down, diluted with DMSO (1 mL) and submitted to reverse phase HPLC (column: XBridge BEH C18 5 um 130 A; mobile phase: 50-50-100% 0-1-5 min H₂O/MeOH/0.1% NH₄OH; flow rate: 30 mL/min (loading pump 4 mL/min methanol) to afford 3 fractions of the product (97-100% purity) N-[4-amino-1-(2-trimethylsilylethoxymethyl)pyrazolo[4,3-c]pyridin-7-yl]-2-oxo-2-[rac-(2R,6R)-2-methyl-6-phenyl-1-piperidyl]acetamide (186 mg, 365.65 μmol, 36.02% yield) as light-brown gum, which were combined and used in the next step.

LCMS(ESI): [M+H]⁺ m/z: calcd 509.2; found 509.2; Rt=3.225 min.

### Step 4: The Synthesis of N-(4-Amino-1H-pyrazolo[4,3-c]pyridin-7-yl)-2-oxo-2-[rac-(2R,6R)-2-methyl-6-phenyl-1-piperidyl]acetamide (Compound 270)

Hydrogen chloride solution 4.0M in dioxane (3.68 g, 14.01 mmol, 3.5 mL, 13.9% purity) was added to a stirred solution of N-[4-amino-1-(2-trimethylsilylethoxymethyl)pyrazolo[4,3-c]pyridin-7-yl]-2-oxo-2-[rac-(2R,6R)-2-methyl-6-phenyl-1-piperidyl]acetamide (186 mg, 365.65 μmol) in methanol (4 mL) at 25° C. The resulting solution was stirred at 25° C. for 15 hr, then concentrated to dryness in vacuo and the residue was submitted to reverse phase HPLC (column: Chromatorex 18 SMB100-5T 100×19 mm 5 um; mobile phase: 5-5-30% 0-1-5 min H₂O/ACN/0.1% FA, flow: 30 mL/min (loading pump 4 mL/min acetonitrile)) to afford Compound 270 N-(4-amino-1H-pyrazolo[4,3-c]pyridin-7-yl)-2-oxo-2-[rac-(2R,6R)-2-methyl-6-phenyl-1-piperidyl]acetamide (101 mg, 237.95 μmol, 65.08% yield, HCOOH) as light-yellow solid.

¹H NMR (600 MHz, dmso) δ 0.19-0.86 (m, 3H), 1.12-1.94 (m, 5H), 2.55-2.59 (m, 1H), 3.97-4.78 (m, 1H), 4.89-5.90 (m, 1H), 6.63-7.20 (m, 3H), 7.22-7.82 (m, 5H), 8.08-8.32 (m, 2H), 9.53-10.66 (m, 1H), 12.54-13.37 (m, 1H).

LCMS(ESI): [M+H]⁺ m/z: calcd 379.2; found 379.2; Rt=1.175 min.

### Example 238. N-(4-Amino-1-methyl-pyrazolo[4,3-c]pyridin-7-yl)-2-oxo-2-[(2S,5S)-2-ethyl-5-methyl-1-piperidyl]acetamide (Compound 281)

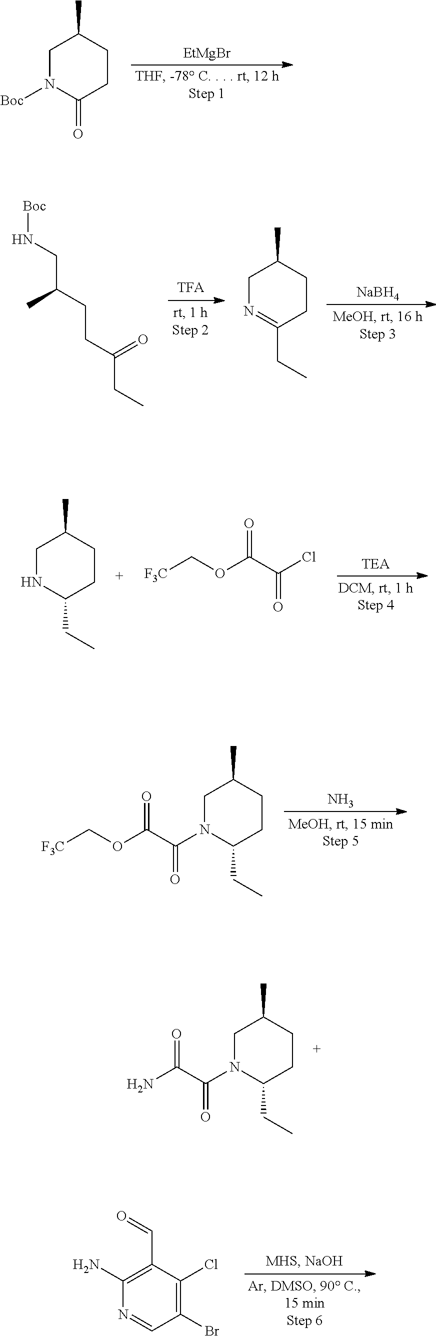

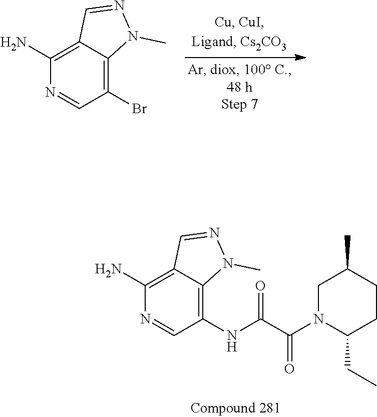 [SMILES: Cn1ncc2c(N)ncc(Br)c21 ; CC[C@H]1CC[C@H](C)CN1C(=O)C(=O)Nc1cnc(N)c2cnn(C)c12]

### Step 1: The Synthesis of tert-Butyl N-[(2S)-2-methyl-5-oxo-heptyl]carbamate

Ethylmagnesium bromide (2.25 g, 16.88 mmol, 1.29 mL) was transferred dropwise to a cold (−78° C.) suspension of tert-butyl (5S)-5-methyl-2-oxo-piperidine-1-carboxylate (3 g, 14.07 mmol) in THF (199.43 mL). The reaction mixture was stirred at −78° C. for 10 min before being warmed to rt and stirred for 12 h. The reaction was diluted with MTBE (300 ml) and quenched with 100 mL of saturated ammonium chloride aqueous solution. The organic phase was washed with a saturated aqueous solution of sodium bicarbonate (2×200 ml). The combined aqueous fractions were back-extracted two times with MTBE. The combined organic fractions were dried over sodium sulfate, and concentrated by rotary evaporation to obtain tert-butyl N-[(2S)-2-methyl-5-oxo-heptyl]carbamate (2.8 g, 11.51 mmol, 81.80% yield) as a pale-yellow gum.

¹H NMR (400 MHZ, CDCl₃) δ (ppm) 0.87 (m, 3H), 1.02 (m, 3H), 1.41 (m, 10H), 1.61 (m, 2H), 2.41 (m, 4H), 2.97 (m, 2H), 4.62 (m, 1H).

### Step 2: The Synthesis of (3S)-6-Ethyl-3-methyl-2,3,4,5-tetrahydropyridine

tert-Butyl N-[(2S)-2-methyl-5-oxo-heptyl]carbamate (2.80 g, 11.51 mmol) was dissolved in TFA (3.94 g, 34.52 mmol, 2.66 mL) and the resulting reaction mixture was stirred for 1 hour. After 1 hour, 50% aq. NaOH solution was added to the reaction mixture till pH=13-14. The resulting mixture was extracted with DCM (4×100 mL). The combined organic phase was dried over MgSO₄ and concentrated under reduced pressure. (3S)-6-Ethyl-3-methyl-2,3,4,5-tetrahydropyridine (1.5 g, crude) was obtained as light-yellow oil.

### Step 3: The Synthesis of (2S,5S)-2-Ethyl-5-methyl-piperidine

To an ice cold solution of (3S)-6-ethyl-3-methyl-2,3,4,5-tetrahydropyridine (1.5 g, 11.98 mmol) in MeOH (15 mL) was added Sodium Borohydride (453.23 mg, 11.98 mmol, 422.00 μL) in one portion and the resulting mixture was left to stir overnight at r.t. The reaction mixture was evaporated, partitioned in mixture EtOAc/water. Water was extracted with EtOAc (10 ml). Combined organics were dried over Na₂SO₄ and evaporated to give (2S,5S)-2-ethyl-5-methyl-piperidine (1.5 g, 11.79 mmol, 98.42% yield) as a light-yellow oil.

### Step 4: The Synthesis of 2,2,2-Trifluoroethyl 2-oxo-2-[(2S,5S)-2-ethyl-5-methyl-1-piperidyl]acetate

To a solution of (2S,5S)-2-ethyl-5-methyl-piperidine (0.6 g, 4.72 mmol) and TEA (572.65 mg, 5.66 mmol, 788.78 μL) in DCM (14.72 mL) was added 2,2,2-trifluoroethyl 2-chloro-2-oxo-acetate (988.26 mg, 5.19 mmol) at rt. After stirring at rt for 1 hr the resulting mixture was washed with water, dried, and evaporated to dryness to give 2,2,2-trifluoroethyl 2-oxo-2-[(2S,5S)-2-ethyl-5-methyl-1-piperidyl]acetate (1.2 g, 4.27 mmol, 90.47% yield) as a colorless gum and was used in the next step without further purification.

LCMS(ESI): [M+H]⁺ m/z: calcd 282.2; found 282.2; Rt=3.963 min.

### Step 5: The Synthesis of 2-Oxo-2-[(2S,5S)-2-ethyl-5-methyl-1-piperidyl]acetamide

Ammonia (72.66 mg, 4.27 mmol) was bubbled through a solution of 2,2,2-trifluoroethyl 2-oxo-2-[(2S,5S)-2-ethyl-5-methyl-1-piperidyl]acetate (1.2 g, 4.27 mmol) in MeOH (25 mL) at rt for 15 min. After stirring for 18 hr, the reaction mixture was evaporated to dryness to give 2-oxo-2-[(2S,5S)-2-ethyl-5-methyl-1-piperidyl]acetamide (0.9 g, crude) as a yellow solid.

LCMS(ESI): [M+H]⁺ m/z: calcd 199.4; found 199.4; Rt=2.213 min.

### Step 6: The Synthesis of 7-Bromo-1-methyl-pyrazolo[4,3-c]pyridin-4-amine

2-Amino-5-bromo-4-chloro-pyridine-3-carbaldehyde (1 g, 4.25 mmol), N-methylhydrazine sulfate (918.29 mg, 6.37 mmol) and NaOH (849.38 mg, 21.23 mmol, 398.77 μL) were mixed in DMSO under argon, and then stirred for 15 min at 90° C. The reaction mixture was poured into water, extracted three times with EtOAc, combined organics were washed with water (2×25 mL), brine, dried and evaporated to give a residue which was purified by CC (Interchim; 40 g SiO₂, chloroform/acetonitrile with acetonitrile from 0˜60%, flow rate=40 mL/min, Rv=17-20CV) to give 7-bromo-1-methyl-pyrazolo[4,3-c]pyridin-4-amine (0.1 g, 440.41 μmol, 10.37% yield) as a yellow solid.

LCMS(ESI): [M+H]⁺ m/z: calcd 227.0; found 227.0; Rt=0.699 min.

### Step 7: The Synthesis of N-(4-Amino-1-methyl-pyrazolo[4,3-c]pyridin-7-yl)-2-oxo-2-[(2S,5S)-2-ethyl-5-methyl-1-piperidyl]acetamide (Compound 281)

2-Oxo-2-[(2S,5S)-2-ethyl-5-methyl-1-piperidyl]acetamide (29 mg, 146.27 μmol), 7-bromo-1-methyl-pyrazolo[4,3-c]pyridin-4-amine (50.42 mg, 137.66 μmol), Copper (I) iodide (13.93 mg, 73.14 μmol, 2.48 μL), Cesium carbonate (95.32 mg, 292.54 μmol) and (1R,2R)—N1,N2-dimethylcyclohexane-1,2-diamine (20.81 mg, 146.27 μmol) were mixed in dioxane (5.01 mL) under argon, and then stirred for 72 h at 100° C. for 48 hr in vial. The reaction mixture was filtered and concentrated in vacuo. The residue was purified by HPLC (1st run: 23-30-60-100% 0-2-7-7.1 min; 30 ml/min water-MeOH+nh3 (loading pump 4 ml/min MeOH); target mass 430; column Xbridge C18 5 uM 19*100 mm (L)); 2nd run: 17-25-50-100% 0-2-7-7.1 min; 30 ml/min water-acn (loading pump 4 ml/min acn); target mass 345; column Kinetex PFP 5 μM 21.2*100 mm (R)). N-(4-Amino-1-methyl-pyrazolo[4,3-c]pyridin-7-yl)-2-oxo-2-[(2S,5S)-2-ethyl-5-methyl-1-piperidyl]acetamide (5.6 mg, 16.26 μmol, 11.12% yield) was obtained as a yellow gum. 1st and 2nd fraction were combined with another batch to give final Compound 281 fraction.

¹H NMR (600 MHz, dmso) δ 4.49-4.71 (m, 2H), 4.89-5.17 (m, 2H), 6.90 (s, 2H), 7.10-7.18 (m, 2H), 7.29-7.47 (m, 3H), 7.57-7.73 (m, 1H), 7.80-7.92 (m, 1H), 8.17-8.27 (m, 1H), 8.45-8.55 (m, 1H), 10.23-10.63 (m, 1H), 12.89 (br s, 1H).

LCMS(ESI): [M+H]⁺ m/z: calcd 345.2; found 345.2; Rt=1.905 min.

### Example 239. The Synthesis of N-(4-Amino-1H-pyrazolo[4,3-c]pyridin-7-yl)-2-oxo-2-[(2S,5S)-2-ethyl-5-methyl-1-piperidyl]acetamide (Compound 273)

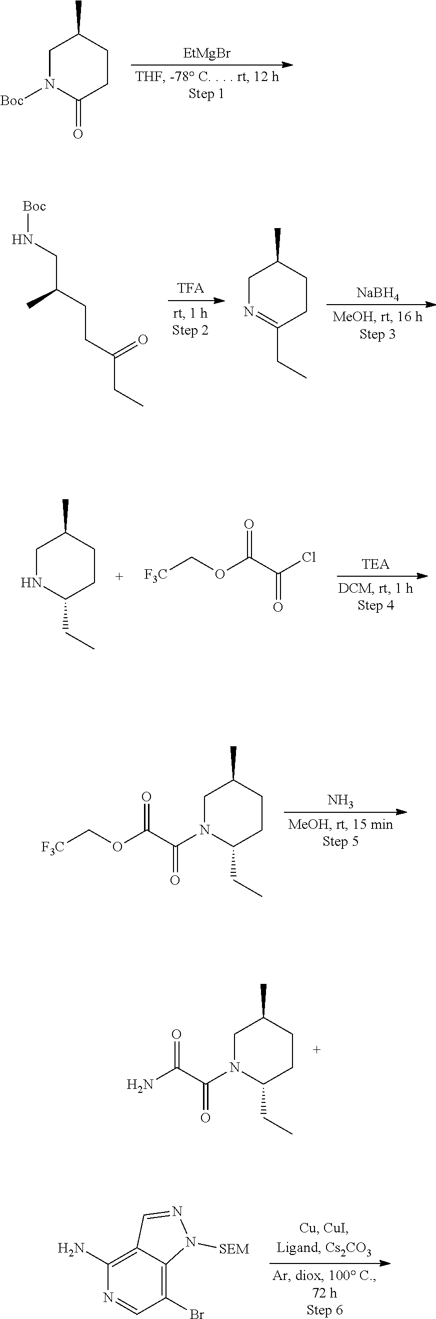

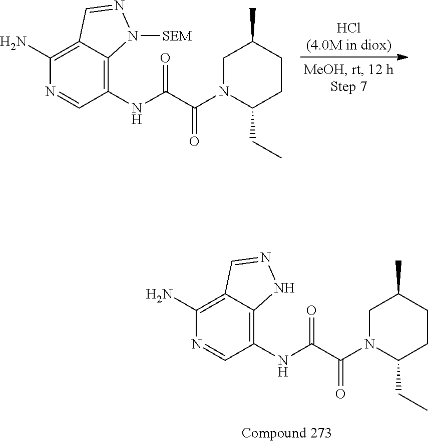 [SMILES: CC[C@H]1CC[C@H](C)CN1C(=O)C(=O)Nc1cnc(N)c2cnn(COCC[Si](C)(C)C)c12 ; "HCl(4.0M in diox)" ; CC[C@H]1CC[C@H](C)CN1C(=O)C(=O)Nc1cnc(N)c2cn[nH]c12]

### Steps 1-5 Described Previously for Compound 281

### Step 6: The Synthesis of N-[4-Amino-1-(2-trimethylsilylethoxymethyl)pyrazolo[4,3-c]pyridin-7-yl]-2-oxo-2-[(2S,5S)-2-ethyl-5-methyl-1-piperidyl]acetamide

2-Oxo-2-[(2S,5S)-2-ethyl-5-methyl-1-piperidyl]acetamide (30 mg, 151.32 μmol), 7-bromo-1-(2-trimethylsilylethoxymethyl)pyrazolo[4,3-c]pyridin-4-amine (51.95 mg, 151.32 μmol), Copper (I) iodide (14.41 mg, 75.66 μmol, 2.56 μL), Cesium carbonate (98.60 mg, 302.63 μmol) and (1R,2R)—N1,N2-dimethylcyclohexane-1,2-diamine (21.52 mg, 151.32 μmol) were mixed in dioxane (5.01 mL) under argon, and then stirred for 72 h at 100° C. for 72 hr in vial. The reaction mixture was filtered and concentrated in vacuo. The residue was combined with another batch upon HPLC purification (40-80% 0.5-6.5 min; 30 ml/min water acn+NH₃ (loading pump 4 ml/min acn); column XBridge 19*100 mm (L)) to give N-[4-amino-1-(2-trimethylsilylethoxymethyl)pyrazolo[4,3-c]pyridin-7-yl]-2-oxo-2-[(2S,5S)-2-ethyl-5-methyl-1-piperidyl]acetamide (35 mg, 75.98 μmol, 50.21% yield) as a yellow solid.

LCMS(ESI): [M+H]⁺ m/z: calcd 463.2; found 463.2; Rt=3.664 min.

### Step 9: The Synthesis of N-(4-Amino-1H-pyrazolo[4,3-c]pyridin-7-yl)-2-oxo-2-[(2S,5S)-2-ethyl-5-methyl-1-piperidyl]acetamide (Compound 273)

To a solution of N-[4-amino-1-(2-trimethylsilylethoxymethyl)pyrazolo[4,3-c]pyridin-7-yl]-2-oxo-2-[(2S,5S)-2-ethyl-5-methyl-1-piperidyl]acetamide (42.7 mg, 92.70 μmol) in meOH (1 mL) was added Hydrogen chloride solution 4.0M in dioxane (800.00 mg, 21.94 mmol, 1 mL) at 21° C. The resulting mixture was left to stir for 12 hr. The resulting mixture was evaporated to dryness and subjected to HPLC (20-40% 2-7 min; 30 ml/min water-acetonitrile+nh3 (loading pump 4 ml/min acetonitrile); column xbridgeC18 19*100 mm (L)). N-(4-amino-1H-pyrazolo[4,3-c]pyridin-7-yl)-2-oxo-2-[(2S,5S)-2-ethyl-5-methyl-1-piperidyl]acetamide (15.6 mg, 47.22 μmol, 50.94% yield) was obtained as a beige solid in 2 fractions: 1st—7.7 mg (98.59% by LCMS); 2nd—7.9 mg (58.83% by LCMS; 41.17% of cis-impurity).

¹H NMR (600 MHz, dmso) δ 0.73-0.86 (m, 3H), 0.91-1.06 (m, 3H), 1.25-1.34 (m, 1H), 1.37-1.47 (m, 1H), 1.51-1.61 (m, 1H), 1.67-2.02 (m, 4H), 2.92-4.43 (m, 3H), 7.38-9.14 (m, 4H), 10.69 (s, 1H), 12.49-13.75 (m, 1H).

LCMS(ESI): [M+H]⁺ m/z: calcd 331.2; found 331.2; Rt=2.155 min.

### Example 240. N-(4-Amino-1H-pyrazolo[4,3-c]pyridin-7-yl)-2-oxo-2-[(2R,5S)-2-cyclopropyl-5-methyl-1-piperidyl]acetamide (Compound 277)

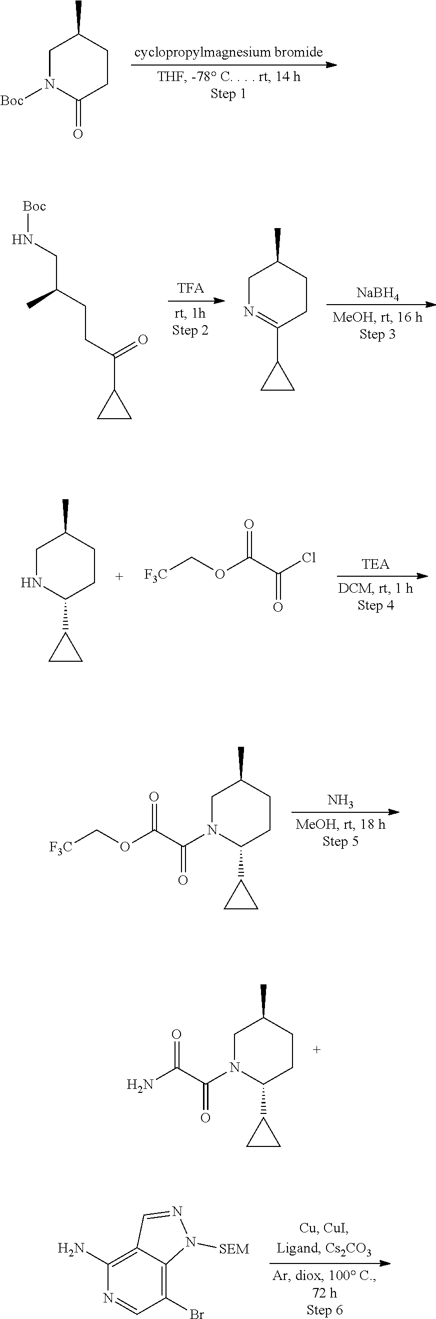

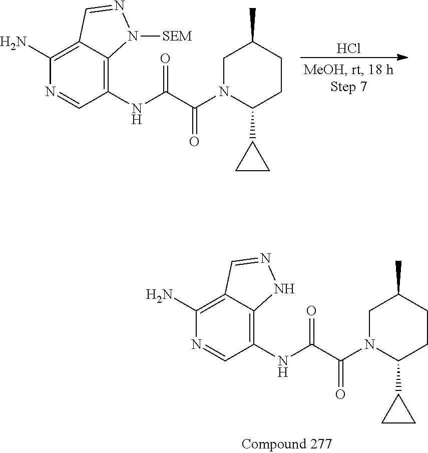 [SMILES: C[C@H]1CC[C@H](C2CC2)N(C(=O)C(=O)Nc2cnc(N)c3cnn(COCC[Si](C)(C)C)c23)C1 ; Cl ; C[C@H]1CC[C@H](C2CC2)N(C(=O)C(=O)Nc2cnc(N)c3cn[nH]c23)C1]

### Step 1: The Synthesis of tert-Butyl N-[(2S)-5-cyclopropyl-2-methyl-5-oxo-pentyl]carbamate

Cyclopropylmagnesium bromide (2.37 g, 16.88 mmol, 1.36 mL) was transferred dropwise to a cold (−78° C.) suspension of tert-butyl (5S)-5-methyl-2-oxo-piperidine-1-carboxylate (3 g, 14.07 mmol) in THF (50 mL). The reaction mixture was stirred at −78° C. for 1 hr before being warmed to rt and stirred for 12 h. The reaction was diluted with MTBE (50 mL) and slowly quenched with 50 mL of saturated ammonium chloride aqueous solution. The organic phase was washed with a saturated aqueous solution of sodium bicarbonate (2×50 mL). The combined aqueous fractions were back-extracted two times with MTBE. The combined organic fractions were dried over sodium sulfate and concentrated by rotary evaporation to obtain crude material that was used in the next step without further purification. tert-Butyl N-[(2S)-5-cyclopropyl-2-methyl-5-oxo-pentyl]carbamate (3.6 g, crude).

¹H NMR (500 MHz, CDCl₃) δ (ppm) 0.88 (m, 5H), 0.99 (m, 2H), 1.43 (m, 11H), 1.60 (m, 1H), 1.90 (m, 1H), 2.57 (m, 2H), 2.99 (m, 2H), 4.67 (m, 1H).

### Step 2: The Synthesis of (3S)-6-Cyclopropyl-3-methyl-2,3,4,5-tetrahydropyridine

tert-Butyl N-[(2S)-5-cyclopropyl-2-methyl-5-oxo-pentyl]carbamate (3.4 g, 13.31 mmol) was dissolved in TFA (4.55 g, 39.94 mmol, 3.08 mL) and the resulting reaction mixture was stirred for 1 hour. After 1 hour, 50% aq. NaOH solution was added to the reaction mixture till pH=13-14. The resulting mixture was extracted with DCM (4×100 mL). The combined organic phase was dried over MgSO₄ and concentrated under reduced pressure. (3S)-6-Cyclopropyl-3-methyl-2,3,4,5-tetrahydropyridine (1.72 g, 12.53 mmol, 94.14% yield) was obtained as brown oil.

¹H NMR (500 MHz, CDCl₃) δ (ppm) 0.67 (m, 3H), 0.89 (d, 3H), 1.19 (m, 1H), 1.48 (m, 1H), 1.71 (m, 2H), 2.14 (m, 2H), 2.93 (m, 1H), 3.65 (m, 1H).

### Step 3: The Synthesis of (2R,5S)-2-Cyclopropyl-5-methyl-piperidine

To an ice-cold solution of (3S)-6-cyclopropyl-3-methyl-2,3,4,5-tetrahydropyridine (1.72 g, 12.53 mmol) in MeOH (49.56 mL) was added Sodium Borohydride (474.21 mg, 12.53 mmol, 441.54 μL) in one portion and the resulting mixture was stirred for 5 min. The reaction mixture was evaporated, partitioned in mixture of DCM/water. Water was extracted with DCM (50 mL). Combined organics were dried over Na₂SO₄ and evaporated to give a residue that was used in the next step without further purification. (2R,5S)-2-Cyclopropyl-5-methyl-piperidine (1.7 g, crude) was obtained as a brown liquid.

### Step 4: The Synthesis of 2,2,2-Trifluoroethyl 2-oxo-2-[(2R,5S)-2-cyclopropyl-5-methyl-1-piperidyl]acetate

To a solution of (2R,5S)-2-cyclopropyl-5-methyl-piperidine (0.6 g, 4.31 mmol) and TEA (523.26 mg, 5.17 mmol, 720.74 μL) in DCM (14.79 mL) was added 2,2,2-trifluoroethyl 2-chloro-2-oxo-acetate (903.01 mg, 4.74 mmol) at rt. After stirring at rt for 1 hr the resulting mixture was washed with water, dried, and evaporated to dryness to give 2,2,2-trifluoroethyl 2-oxo-2-[(2R,5S)-2-cyclopropyl-5-methyl-1-piperidyl]acetate (1.2 g, crude) as a colorless gum and was used in the next step without further purification.

### Step 5: The Synthesis of 2-Oxo-2-[(2R,5S)-2-cyclopropyl-5-methyl-1-piperidyl]acetamide

Ammonia (69.68 mg, 4.09 mmol) was bubbled through a solution of 2,2,2-trifluoroethyl 2-oxo-2-[(2R,5S)-2-cyclopropyl-5-methyl-1-piperidyl]acetate (1.2 g, 4.09 mmol) in MeOH (25 mL) at rt. After stirring for 18 hr, the reaction mixture was evaporated to dryness to give 2-oxo-2-[(2R,5S)-2-cyclopropyl-5-methyl-1-piperidyl]acetamide (0.9 g, crude) as a yellow gum.

LCMS(ESI): [M+H]⁺ m/z: calcd 211.4; found 211.4; Rt=2.444 min.

### Step 6: The Synthesis of N-[4-Amino-1-(2-trimethylsilylethoxymethyl)pyrazolo[4,3-c]pyridin-7-yl]-2-oxo-2-[(2R,5S)-2-cyclopropyl-5-methyl-1-piperidyl]acetamide

2-Oxo-2-[(2R,5S)-2-cyclopropyl-5-methyl-1-piperidyl]acetamide (150 mg, 713.36 μmol), 7-bromo-1-(2-trimethylsilylethoxymethyl)pyrazolo[4,3-c]pyridin-4-amine (244.89 mg, 713.36 μmol), Copper (I) iodide (67.93 mg, 356.68 μmol, 12.09 μL), Cesium carbonate (464.85 mg, 1.43 mmol) and (1R,2R)—N1,N2-dimethylcyclohexane-1,2-diamine (101.47 mg, 713.36 μmol) were mixed in dioxane (5.00 mL) under argon, and then stirred for 72 h at 100° C. for 72 h in vial. The reaction mixture was filtered and concentrated in vacuo. The residue was purified by HPLC (40-65% 0.5-6.5 min; 30 mL/min water-MeCN+NH₃ (loading pump 4 mL/min MeCN); column XBridge 19*100 mm (L)). N-[4-Amino-1-(2-trimethylsilylethoxymethyl)pyrazolo[4,3-c]pyridin-7-yl]-2-oxo-2-[(2R,5S)-2-cyclopropyl-5-methyl-1-piperidyl]acetamide (25.4 mg, 53.74 μmol, 7.53% yield) was obtained as a beige solid.

### Step 7: The Synthesis of N-(4-Amino-1H-pyrazolo[4,3-c]pyridin-7-yl)-2-oxo-2-[(2R,5S)-2-cyclopropyl-5-methyl-1-piperidyl]acetamide (Compound 277)

To a solution of N-[4-amino-1-(2-trimethylsilylethoxymethyl)pyrazolo[4,3-c]pyridin-7-yl]-2-oxo-2-[(2R,5S)-2-cyclopropyl-5-methyl-1-piperidyl]acetamide (25.4 mg, 38.69 μmol) in MeOH (2 mL) was added Hydrogen chloride solution 4.0M in dioxane (1.60 g, 43.88 mmol, 2 mL) at 21° C. The resulting mixture was left to stir for 18 hr. The resulting mixture was evaporated to dryness and subjected to HPLC (10-30% 2-7 min; 30 mL/min water-acetonitrile+NH₃ (loading pump 4 mL/min acetonitrile); column xbridge C18 19*100 mm (L)). N-(4-Amino-1H-pyrazolo[4,3-c]pyridin-7-yl)-2-oxo-2-[(2R,5S)-2-cyclopropyl-5-methyl-1-piperidyl]acetamide (7 mg, 20.44 μmol, 52.84% yield) was obtained as a beige solid.

¹H NMR (600 MHZ, dmso) δ 0.18-0.40 (m, 2H), 0.40-0.60 (m, 2H), 0.81-1.01 (m, 3H), 1.22-1.83 (m, 4H), 1.83-1.98 (m, 1H), 1.99-2.10 (m, 1H), 2.81-3.21 (m, 1H), 3.36-4.12 (m, 2H), 6.79 (s, 2H), 7.39-7.76 (m, 1H), 8.17 (d, 1H), 9.34-10.43 (m, 1H), 12.49-13.37 (m, 1H).

LCMS(ESI): [M+H]⁺ m/z: calcd 343.2; found 343.2; Rt=1.892 min.

### Example 241. N-(4-Amino-1H-pyrazolo[4,3-c]pyridin-7-yl)-2-oxo-2-[(2R,5S)-2-[2-[3-(dimethylamino) propyl]-1,3-benzothiazol-5-yl]-5-methyl-1-piperidyl]acetamide (Compound 278)

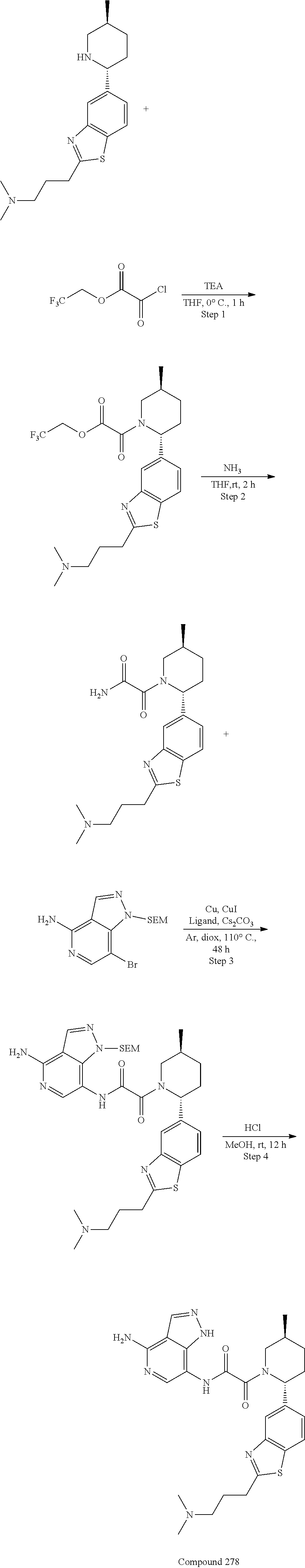

### Step 1: The Synthesis of 2,2,2-Trifluoroethyl 2-oxo-2-[(2R,5S)-2-[2-[3-(dimethylamino) propyl]-1,3-benzothiazol-5-yl]-5-methyl-1-piperidyl]acetate

2,2,2-Trifluoroethyl 2-chloro-2-oxo-acetate (216.01 mg, 1.13 mmol) was added dropwise to a stirred solution of N,N-dimethyl-3-[5-[(2R,5S)-5-methyl-2-piperidyl]-1,3-benzothiazol-2-yl]propan-1-amine (0.3 g, 944.91 μmol) and TEA (191.23 mg, 1.89 mmol, 263.40 μL) in THF (15.25 mL) at 0° C., stirred for 1 hr at 0° C. Reaction mixture was used in the next step.

LCMS(ESI): [M+H]⁺ m/z: calcd 472.2; found 472.2; Rt=1.157 min.

### Step 2: The Synthesis of 2-Oxo-2-[(2R,5S)-2-[2-[3-(dimethylamino) propyl]-1,3-benzothiazol-5-yl]-5-methyl-1-piperidyl]acetamide

Ammonia (317.83 mg, 18.66 mmol) was bubbled trough a reaction mixture at 0° C., stirred for 1 hr at 0° C. and 2 hr at 25° C. Reaction mixture was filtered, solid washed with THF (2×5 mL), filtrate evaporated in vacuo to give 2-oxo-2-[(2R,5S)-2-[2-[3-(dimethylamino) propyl]-1,3-benzothiazol-5-yl]-5-methyl-1-piperidyl]acetamide (0.28 g, 720.67 μmol, 77.23% yield).

LCMS(ESI): [M+H]⁺ m/z: calcd 472.2; found 472.2; Rt=0.823 min.

### Step 3: The Synthesis of N-[4-Amino-1-(2-trimethylsilylethoxymethyl)pyrazolo[4,3-c]pyridin-7-yl]-2-oxo-2-[(2R,5S)-2-[2-[3-(dimethylamino) propyl]-1,3-benzothiazol-5-yl]-5-methyl-1-piperidyl]acetamide

Copper (2.29 mg, 36.03 μmol), Copper (I) iodide (68.63 mg, 360.34 μmol, 12.21 μL), caesium carbonate (469.62 mg, 1.44 mmol) was added to a stirred solution of 2-oxo-2-[(2R,5S)-2-[2-[3-(dimethylamino) propyl]-1,3-benzothiazol-5-yl]-5-methyl-1-piperidyl]acetamide (0.28 g, 720.67 μmol), 7-bromo-1-(2-trimethylsilylethoxymethyl)pyrazolo[4,3-c]pyridin-4-amine (259.77 mg, 756.71 μmol), rac-(1R,2R)—N1,N2-dimethylcyclohexane-1,2-diamine (102.51 mg, 720.67 μmol) in 1,4-dioxane (7.00 mL) under Ar atmosphere and stirred at 110° C. for 48 hr in closed vial. Reaction mixture was filtered, solid washed with dioxane (2×3 mL), filtrate concentrated. The residue was mixed with TBME (10 mL), filtered, washed with hexane (10 mL) to give crude N-[4-amino-1-(2-trimethylsilylethoxymethyl)pyrazolo[4,3-c]pyridin-7-yl]-2-oxo-2-[rac-(2R,5S)-2-[2-[3-(dimethylamino) propyl]-1,3-benzothiazol-5-yl]-5-methyl-1-piperidyl]acetamide (0.15 g, 230.45 μmol, 31.98% yield).

LCMS(ESI): [M+H]⁺ m/z: calcd 651.2; found 651.2; Rt=1.113 min.

### Step 4: The Synthesis of N-(4-Amino-1H-pyrazolo[4,3-c]pyridin-7-yl)-2-oxo-2-[rac-(2R,5S)-2-[2-[3-(dimethylamino) propyl]-1,3-benzothiazol-5-yl]-5-methyl-1-piperidyl]acetamide (Compound 278)

Hydrogen chloride solution 4.0M in dioxane (1.26 g, 34.57 mmol, 1.58 mL) was added to a solution of N-[4-amino-1-(2-trimethylsilylethoxymethyl)pyrazolo[4,3-c]pyridin-7-yl]-2-oxo-2-[rac-(2R,5S)-2-[2-[3-(dimethylamino) propyl]-1,3-benzothiazol-5-yl]-5-methyl-1-piperidyl]acetamide (0.15 g, 230.45 μmol) in Methanol (454.37 μL) and stirred at 25° C. for 12 hr. Volatiles was evaporated in vacuo, residue dissolved in Methanol (2 mL), neutralized with TEA (60 mg) and submitted to HPLC (column: Chromatorex 18 SMB 100-ST 100*19 mm 5 um; 5-5-25% 0-1-5 min H₂O/ACN/0.1% FA, flow rate: 30 mL/min) to give N-(4-amino-1H-pyrazolo[4,3-c]pyridin-7-yl)-2-oxo-2-[rac-(2R,5S)-2-[2-[3-(dimethylamino) propyl]-1,3-benzothiazol-5-yl]-5-methyl-1-piperidyl]acetamide (3.7 mg, 6.64 μmol, 2.88% yield, HCl).

¹H NMR (600 MHz, dmso) δ 0.91-1.14 (m, 3H), 1.19-1.46 (m, 1H), 1.68-1.80 (m, 1H), 1.85-1.96 (m, 3H), 2.07-2.13 (m, 1H), 2.14 (s, 6H), 2.29-2.32 (m, 2H), 2.76-2.85 (m, 1H), 3.07-3.11 (m, 2H), 3.71-4.16 (m, 1H), 5.12-5.78 (m, 1H), 6.59-6.99 (m, 2H), 7.35-7.47 (m, 1H), 7.57-7.74 (m, 1H), 7.84-7.93 (m, 1H), 7.95-8.07 (m, 1H), 8.09-8.22 (m, 2H), 9.67-10.58 (m, 1H), 12.69-13.44 (m, 1H).

LCMS(ESI): [M+H]⁺ m/z: calcd 521.2; found 521.2; Rt=2.133 min.

### Example 242. N-(4-Amino-2H-pyrazolo[4,3-c]pyridin-7-yl)-2-oxo-2-[rac-(5S)-5-methyl-2-(2-methyl-3,4-dihydro-1H-isoquinolin-7-yl)-1-piperidyl]acetamide (Compound 279)

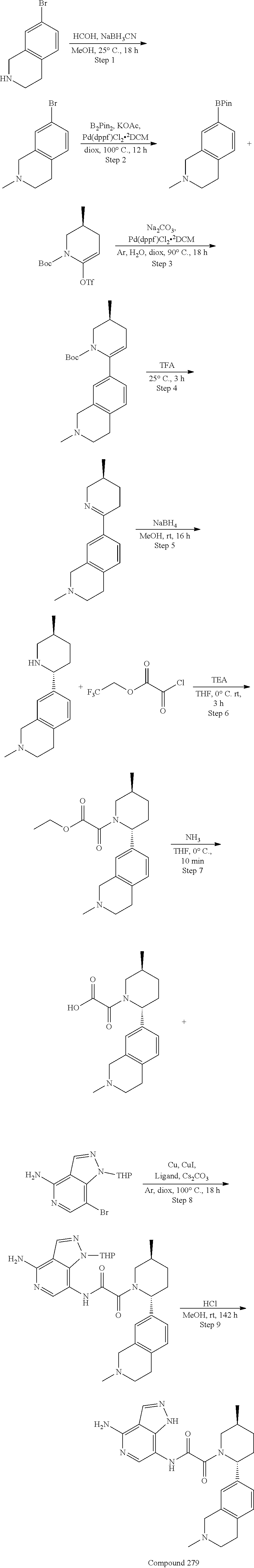

### Step 1: The Synthesis of 7-Bromo-2-methyl-3,4-dihydro-1H-isoquinoline

To a solution of 7-bromo-1,2,3,4-tetrahydroisoquinoline (10 g, 40.23 mmol, HCl) and Formaldehyde, 37% in aq. soln., ACS, 36.5-38.0%, stab. with 10-15% methanol (1.81 g, 60.35 mmol, 1.67 mL) in MeOH (80 mL), Sodium cyanoborohydride (3.03 g, 48.28 mmol, 252.84 μL) was added. The resulting mixture was stirred at 25° C. for 18 hr, the solid was filtered off and the solvent was removed in vacuo. The residue was diluted with water (50 mL), pH of the solution was adjusted to 10 with NaOH and extracted with DCM (3*50 mL). The combined organic phase was dried over Na₂SO₄ and evaporated in vacuo. The residue was purified by gradient chromatography (MTBE-MeOH) to give 7-bromo-2-methyl-3,4-dihydro-1H-isoquinoline (6.2 g, 27.42 mmol, 68.15% yield).

### Step 2: The Synthesis of 2-Methyl-7-(4,4,5,5-tetramethyl-1,3,2-dioxaborolan-2-yl)-3,4-dihydro-1H-isoquinoline

A solution of 7-bromo-2-methyl-3,4-dihydro-1H-isoquinoline (6.2 g, 27.42 mmol), Octamethyl-2,2′-bi-1,3,2-dioxaborolane (7.66 g, 30.16 mmol), Potassium acetate (4.04 g, 41.13 mmol, 2.57 mL) and Pd(dppf)Cl₂·CH₂Cl₂ (1.12 g, 1.37 mmol) in Dioxane (50 mL) was heated at 100° C. for 12 hr. Aliquot showed conversion of SM. Reaction mixture was filtered and directly used for the next step without further treatment.

LCMS(ESI): [M+H]⁺ m/z: calcd 274.2; found 274.2; Rt=0.809 min.

### Step 3: The Synthesis of tert-Butyl rac-(3S)-3-methyl-6-(2-methyl-3,4-dihydro-1H-isoquinolin-7-yl)-3,4-dihydro-2H-pyridine-1-carboxylate

To a solution of 2-methyl-7-(4,4,5,5-tetramethyl-1,3,2-dioxaborolan-2-yl)-3,4-dihydro-1H-isoquinoline (7.4 g, 27.09 mmol), tert-butyl rac-(3S)-3-methyl-6-(trifluoromethylsulfonyloxy)-3,4-dihydro-2H-pyridine-1-carboxylate (11.23 g, 32.51 mmol), Sodium Carbonate (8.61 g, 81.27 mmol, 3.40 mL) in Dioxane (120 mL) and Water (30 mL), Pd(dppf)Cl₂·CH₂Cl₂ (1.11 g, 1.35 mmol) was added under Argon atmosphere. The resulting mixture was stirred at 100° C. for 18 hr and cooled. The organic layer was separated, evaporated, the residue was dissolved in MTBE (500 mL), dried over Na₂SO₄, filtered and evaporated in vacuo to give tert-butyl rac-(3S)-3-methyl-6-(2-methyl-3,4-dihydro-1H-isoquinolin-7-yl)-3,4-dihydro-2H-pyridine-1-carboxylate (9 g, 26.28 mmol, 97.01% yield). Crude substance was used for the next step without further purification.

LCMS(ESI): [M+H]⁺ m/z: calcd 343.2; found 343.2; Rt=0.911 min.

### Step 4: The Synthesis of 2-Methyl-7-[rac-(3S)-3-methyl-2,3,4,5-tetrahydropyridin-6-yl]-3,4-dihydro-1H-isoquinoline

A solution of tert-butyl rac-(3S)-3-methyl-6-(2-methyl-3,4-dihydro-1H-isoquinolin-7-yl)-3,4-dihydro-2H-pyridine-1-carboxylate (9 g, 26.28 mmol) in Trifluoroacetic acid (35 g, 306.96 mmol, 25 mL) was stirred at 25° C. for 3 hr. The solvent was evaporated, and the residue was diluted with water (50 ml). The solution was washed with MTBE (50 ml). pH of the aqueous layer was adjusted to 9-10 with NaOH solution, extracted with DCM (3*30 mL), dried over Na₂SO₄ and evaporated to give 2-methyl-7-[rac-(3S)-3-methyl-2,3,4,5-tetrahydropyridin-6-yl]-3,4-dihydro-1H-isoquinoline (6 g, 24.76 mmol, 94.21% yield).

LCMS(ESI): [M+H]⁺ m/z: calcd 243.2; found 243.2; Rt=0.284 min.

### Step 5: The Synthesis of 2-Methyl-7-[rac-(5S)-5-methyl-2-piperidyl]-3,4-dihydro-1H-isoquinoline

Sodium Borohydride (936.61 mg, 24.76 mmol, 872.08 μL) was added in portions to a stirred solution of 2-methyl-7-[rac-(3S)-3-methyl-2,3,4,5-tetrahydropyridin-6-yl]-3,4-dihydro-1H-isoquinoline (6 g, 24.76 mmol) in Methanol (70 mL) at 0° C. The resulting mixture was stirred at 0° C. for 2 hr, and then evaporated in vacuo. The residue was diluted with water (20 mL) and extracted with dichloromethane (2*40 mL). The combined organic extracts were dried over sodium sulphate and evaporated in vacuo to afford 2-methyl-7-[rac-(5S)-5-methyl-2-piperidyl]-3,4-dihydro-1H-isoquinoline (5 g, 20.46 mmol, 82.65% yield) as yellow oil, which was used directly in the next step.

LCMS(ESI): [M+H]⁺ m/z: calcd 245.2; found 245.2; Rt=0.574 min.

### Step 6: The Synthesis of 2,2,2-Trifluoroethyl 2-oxo-2-[rac-(5S)-5-methyl-2-(2-methyl-3,4-dihydro-1H-isoquinolin-7-yl)-1-piperidyl]acetate

To a solution of 2-methyl-7-[rac-(5S)-5-methyl-2-piperidyl]-3,4-dihydro-1H-isoquinoline (3 g, 12.28 mmol) and Triethylamine (3.73 g, 36.83 mmol, 5.13 mL) in THF (80 mL), 2,2,2-trifluoroethyl 2-chloro-2-oxo-acetate (4.68 g, 24.55 mmol) was added portionwise at 0° C. The resulting mixture was stirred for 3 hr. LCMS showed full conversion of SM. The reaction mixture was directly used for the next step.

LCMS(ESI): [M+H]⁺ m/z: calcd 399.2; found 399.2; Rt=0.617 min.

### Step 7: The Synthesis of 2-Oxo-2-[rac-(5S)-5-methyl-2-(2-methyl-3,4-dihydro-1H-isoquinolin-7-yl)-1-piperidyl]acetamide

Through a solution of 2,2,2-trifluoroethyl 2-oxo-2-[rac-(5S)-5-methyl-2-(2-methyl-3,4-dihydro-1H-isoquinolin-7-yl)-1-piperidyl]acetate (4.8 g, 12.05 mmol) in THF (100 mL), ammonia was bubbled during 10 min at 0° C. The formed precipitate was filtered off, washed with THF (30 mL) and the solvent was evaporated in vacuo to give crude product (6 g), which was purified by gradient chromatography (MTBE-MeOH) 2-oxo-2-[rac-(5S)-5-methyl-2-(2-methyl-3,4-dihydro-1H-isoquinolin-7-yl)-1-piperidyl]acetamide (2 g, 6.34 mmol, 52.63% yield).

LCMS(ESI): [M+H]⁺ m/z: calcd 316.2; found 316.2; Rt=0.799 min.

### Step 8: The Synthesis of N-(4-Amino-2-tetrahydropyran-2-yl-pyrazolo[4,3-c]pyridin-7-yl)-2-oxo-2-[rac-(5S)-5-methyl-2-(2-methyl-3,4-dihydro-1H-isoquinolin-7-yl)-1-piperidyl]acetamide

To a mixture of 2-oxo-2-[rac-(5S)-5-methyl-2-(2-methyl-3,4-dihydro-1H-isoquinolin-7-yl)-1-piperidyl]acetamide (0.35 g, 1.11 mmol), 7-bromo-2-tetrahydropyran-2-yl-pyrazolo[4,3-c]pyridin-4-amine (494.61 mg, 1.66 mmol), Copper (14.10 mg, 221.93 μmol), Copper (I) iodide (211.34 mg, 1.11 mmol, 37.60 μL), Cesium carbonate (723.11 mg, 2.22 mmol) and rac-(1R,2R)—N1,N2-dimethylcyclohexane-1,2-diamine (236.76 mg, 1.66 mmol), Dioxane (4.99 mL) was added. The resulting mixture was evacuated, refiled with Argon three time, heated at 100° C. for 18 hr and cooled. The precipitate was filtered through a thin layer of SiO₂ and washed with DCM (30 ml). The solvent was evaporated to give N-(4-amino-2-tetrahydropyran-2-yl-pyrazolo[4,3-c]pyridin-7-yl)-2-oxo-2-[rac-(5S)-5-methyl-2-(2-methyl-3,4-dihydro-1H-isoquinolin-7-yl)-1-piperidyl]acetamide (0.5 g, 940.47 μmol, 84.75% yield), which was used for the next step without purification.

LCMS(ESI): [M+H]⁺ m/z: calcd 532.2; found 532.2; Rt=0.833 min.

### Step 9: The Synthesis of N-(4-Amino-2H-pyrazolo[4,3-c]pyridin-7-yl)-2-oxo-2-[rac-(5S)-5-methyl-2-(2-methyl-3,4-dihydro-1H-isoquinolin-7-yl)-1-piperidyl]acetamide (Compound 279)

To a solution of N-(4-amino-2-tetrahydropyran-2-yl-pyrazolo[4,3-c]pyridin-7-yl)-2-oxo-2-[rac-(5S)-5-methyl-2-(2-methyl-3,4-dihydro-1H-isoquinolin-7-yl)-1-piperidyl]acetamide (0.5 g, 940.47 μmol) in Methanol (20 mL), Hydrogen chloride solution 4.0M in dioxane (5 g, 137.13 mmol, 6.25 mL) was added. The resulting mixture was stirred at 25° C. for 12 hr and evaporated in vacuo. The residue was purified by HPLC (Device (Mobile Phase, Column): SYSTEM 0-0-20% 0-2-3 min H₂O/ACN/0.2% FA, flow: 30 mL/min (loading pump 4 mL/min acetonitrile); column: Chromatorex 18 SMB100-5T 100×19 mm 5 um) to obtain N-(4-amino-2H-pyrazolo[4,3-c]pyridin-7-yl)-2-oxo-2-[rac-(5S)-5-methyl-2-(2-methyl-3,4-dihydro-1H-isoquinolin-7-yl)-1-piperidyl]acetamide (125 mg, 253.26 μmol, 26.93% yield, HCO₂H).

¹H NMR (600 MHz, dmso) δ 0.96-1.07 (m, 3H), 1.27-1.39 (m, 1H), 1.61-1.69 (m, 1H), 1.83-1.94 (m, 1H), 1.95-2.12 (m, 1H), 2.17-2.25 (m, 1H), 2.75-2.80 (m, 0.4H), 2.85-2.88 (m, 3H), 2.99-3.17 (m, 3H), 3.22-3.24 (m, 0.6H), 3.39-3.42 (m, 1H), 3.64-4.04 (m, 1H), 4.24-4.55 (m, 2H), 5.22-5.66 (m, 1H), 7.17-7.30 (m, 3H), 7.85-8.06 (m, 1H), 8.06-8.98 (m, 3H), 10.94-11.16 (m, 1H), 13.95 (br s, 1H).

LCMS(ESI): [M+H]⁺ m/z: calcd 448.2; found 448.2; Rt=1.438 min.

### Example 243. N-(4-Amino-1H-pyrazolo[4,3-c]pyridin-7-yl)-2-oxo-2-[rac-(2S,5S)-2-(2-isopropylpyrazol-3-yl)-5-methyl-1-piperidyl]acetamide (Compound 280) and N-(4-Amino-1H-pyrazolo[4,3-c]pyridin-7-yl)-2-oxo-2-[rac-(2R,5R)-2-(2-isopropylpyrazol-3-yl)-5-methyl-1-piperidyl]acetamide (Compound 234)

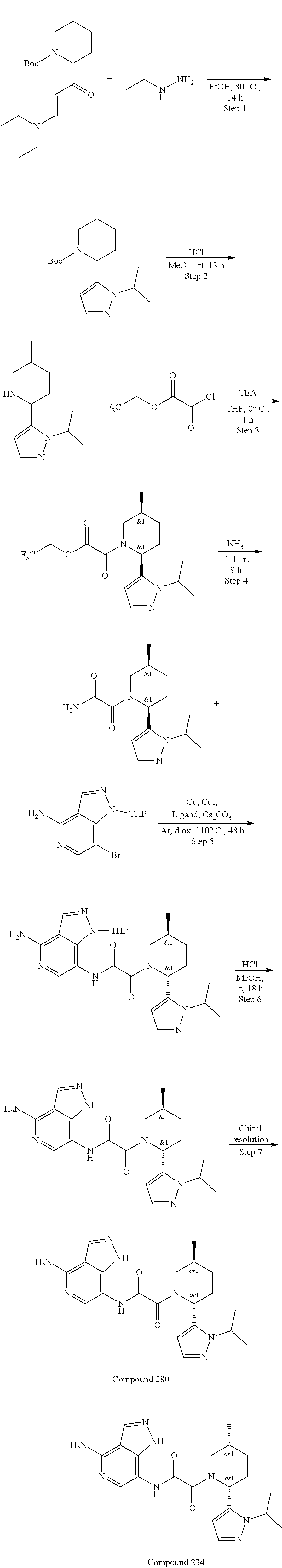

The synthesis of tert-butyl 2-(2-ethylpyrazol-3-yl)-5-methyl-piperidine-1-carboxylate is given in document: Compound 272, Compound 250.

### Step 1: The Synthesis of tert-Butyl 2-(2-isopropylpyrazol-3-yl)-5-methyl-piperidine-1-carboxylate

Isopropylhydrazine (1.91 g, 17.26 mmol, HCl) was added to a stirred solution of tert-butyl 2-[(E)-3-(diethylamino) prop-2-enoyl]-5-methyl-piperidine-1-carboxylate (3.5 g, 10.79 mmol) in Ethanol (40 mL). The reaction mixture was stirred with reflux condenser at 80° C. for 14 hr, then cooled and evaporated in vacuo. The residue was combined with residue from another batch, diluted with water (100 mL) and extracted with MTBE (2*100 mL). The combined organic extracts were washed with water (50 mL), dried over sodium sulphate and evaporated in vacuo to give tert-butyl 2-(2-isopropylpyrazol-3-yl)-5-methyl-piperidine-1-carboxylate (3.3 g, crude).

LCMS(ESI): [M+H]⁺ m/z: calcd 308.2; found 308.2; Rt=1.571 min.

### Step 2: The Synthesis of 2-(2-Isopropylpyrazol-3-yl)-5-methyl-piperidine

Hydrogen chloride solution 4.0M in dioxane (8.00 g, 219.41 mmol, 10 mL) was added to a stirred solution of tert-butyl 2-(2-isopropylpyrazol-3-yl)-5-methyl-piperidine-1-carboxylate (3.3 g, 10.73 mmol) in Methanol (20 mL). The reaction mixture was stirred at 25° C. for 13 hr, and then evaporated in vacuo. The residue was diluted with MTBE (50 mL) then filtered, washed with MTBE (50 mL) and dried in vacuo to give 2-(2-isopropylpyrazol-3-yl)-5-methyl-piperidine (2.4 g, 8.56 mmol, 79.78% yield, 2HCl).

LCMS(ESI): [M+H]⁺ m/z: calcd 208.2; found 208.2; Rt=0.774 min.

### Step 3: The Synthesis of rac-2,2,2-Trifluoroethyl 2-((2R,5R)-2-(1-isopropyl-1H-pyrazol-5-yl)-5-methylpiperidin-1-yl)-2-oxoacetate

2,2,2-Trifluoroethyl 2-chloro-2-oxo-acetate (407.88 mg, 2.14 mmol) was added dropwise to a stirred solution of rac-(2S,5S)-2-(2-isopropylpyrazol-3-yl)-5-methyl-piperidine (0.5 g, 1.78 mmol, 2HCl) and TEA (631.90 mg, 6.24 mmol, 870.39 μL) in THF (29.86 mL) at 0° C., stirred for 1 hr at 0° C. Reaction mixture was used in the next step.

LCMS(ESI): [M+H]⁺ m/z: calcd 362.2; found 362.2; Rt=1.436 min.

### Step 4: The Synthesis of 2-Oxo-2-[rac-(2S,5S)-2-(2-isopropylpyrazol-3-yl)-5-methyl-1-piperidyl]acetamide

Ammonia (784.51 mg, 46.07 mmol) was bubbled trough a reaction mixture at 20° C., stirred for 1 hr at 20° C. and 8 hr at 25° C. Reaction mixture was filtered, solid washed with THF (2×5 mL), filtrate concentrated in vacuo to give pure 2-oxo-2-[rac-(2S,5S)-2-(2-isopropylpyrazol-3-yl)-5-methyl-1-piperidyl]acetamide (0.2 g, 718.52 μmol, 31.20% yield).

LCMS(ESI): [M+H]⁺ m/z: calcd 279.2; found 279.2; Rt=1.059 min.

### Step 5: The Synthesis of N-(4-Amino-1-tetrahydropyran-2-yl-pyrazolo[4,3-c]pyridin-7-yl)-2-oxo-2-[rac-(2R,5S)-2-(2-isopropylpyrazol-3-yl)-5-methyl-1-piperidyl]acetamide

Copper (2.28 mg, 35.93 μmol), Copper (I) iodide (68.42 mg, 359.26 μmol, 12.17 μL), caesium carbonate (351.16 mg, 1.08 mmol) was added to a stirred solution of 2-oxo-2-[rac-(2R,5S)-2-(2-isopropylpyrazol-3-yl)-5-methyl-1-piperidyl]acetamide (0.2 g, 718.52 μmol), 7-bromo-1-tetrahydropyran-2-yl-pyrazolo[4,3-c]pyridin-4-amine (234.86 mg, 790.37 μmol), rac-(1R,2R)—N1,N2-dimethylcyclohexane-1,2-diamine (51.10 mg, 359.26 μmol) in 1,4-dioxane (6.99 mL) under Ar atmosphere and stirred at 110° C. for 48 hr in closed vial. Reaction mixture was filtered, solid washed with dioxane (2×3 mL), filtrate concentrated to give crude N-(4-amino-1-tetrahydropyran-2-yl-pyrazolo[4,3-c]pyridin-7-yl)-2-oxo-2-[rac-(2R,5S)-2-(2-isopropylpyrazol-3-yl)-5-methyl-1-piperidyl]acetamide (0.35 g, 707.66 μmol, 98.49% yield).

LCMS(ESI): [M+H]⁺ m/z: calcd 495.2; found 495.2; Rt=1.132 min.

### Step 6: The Synthesis of N-(4-Amino-1H-pyrazolo[4,3-c]pyridin-7-yl)-2-oxo-2-[rac-(2S,5S)-2-(2-isopropylpyrazol-3-yl)-5-methyl-1-piperidyl]acetamide

Hydrogen chloride solution 4.0M in dioxane (1.35 g, 37.11 mmol, 1.69 mL) was added to a solution of N-(4-amino-1-tetrahydropyran-2-yl-pyrazolo[4,3-c]pyridin-7-yl)-2-oxo-2-[rac-(2S,5S)-2-(2-isopropylpyrazol-3-yl)-5-methyl-1-piperidyl]acetamide (183.53 mg, 371.08 μmol) in Methanol (6.25 mL) and stirred at 20° C. for 18 hr. Volatiles was evaporated in vacuo and the residue submitted to HPLC (column: Chromatorex 18 SMB100-5T 100×19 mm 5 um; 0-5-25% 0-1-6 min H₂O/ACN/0.1% FA, flow rate: 30 mL/min) to give N-(4-amino-1H-pyrazolo[4,3-c]pyridin-7-yl)-2-oxo-2-[rac-(2S,5S)-2-(2-isopropylpyrazol-3-yl)-5-methyl-1-piperidyl]acetamide (58 mg, 141.30 μmol, 38.08% yield).

LCMS(ESI): [M+H]⁺ m/z: calcd 411.2; found 411.2; Rt=2.454 min.

### Step 7: The Synthesis of N-(4-Amino-1H-pyrazolo[4,3-c]pyridin-7-yl)-2-oxo-2-[rac-(2S,5S)-2-(2-isopropylpyrazol-3-yl)-5-methyl-1-piperidyl]acetamide (Compound 280) and N-(4-Amino-1H-pyrazolo[4,3-c]pyridin-7-yl)-2-oxo-2-[rac-(2R,5R)-2-(2-isopropylpyrazol-3-yl)-5-methyl-1-piperidyl]acetamide (Compound 234)

Racemic N-(4-amino-1H-pyrazolo[4,3-c]pyridin-7-yl)-2-oxo-2-[rac-(2S,5S)-2-(2-isopropylpyrazol-3-yl)-5-methyl-1-piperidyl]acetamide (58.0 mg, 141.30 μmol) was separated (Column: CHIRALPAK IA (250×30 mm, 10 mkm); Mobile Phase:

Hexane:IPA:MeOH:DEA, 70:15:15; Flow Rate: 35 mL/min V (mobile phase)=2.2 L/1.7 h/4 injections) affording N-(4-amino-1H-pyrazolo[4,3-c]pyridin-7-yl)-2-oxo-2-[rac-(2S,5S)-2-(2-isopropylpyrazol-3-yl)-5-methyl-1-piperidyl]acetamide (Rt1 (Compound 280)=12.439 min; 16.9 mg, 41.17 μmol, 29.14% yield) and N-(4-amino-1H-pyrazolo[4,3-c]pyridin-7-yl)-2-oxo-2-[rac-(2R,5R)-2-(2-isopropylpyrazol-3-yl)-5-methyl-1-piperidyl]acetamide (Rt2 (Compound 234)=16.412 min; 15.7 mg, 38.25 μmol, 27.07% yield).

### Compound 280:

Preparative: RT (Column: CHIRALPAK IA (250×30 mm, 10 mkm); Mobile Phase:

Hexane:IPA:MeOH:DEA, 70:15:15; Flow Rate: 35 mL/min)=12.266 min.

Analytical: RT (Column: Chiralpak IA (250×4.6 mm, 5 mkm)-1; Mobile Phase: Hexane (0.1% EDA):IPA:MeOH, 70:15:15; Flow Rate: 0.6 mL/min)=12.401 min.

¹H NMR (600 MHz, dmso) δ 0.69-0.92 (m, 3H), 1.01-1.40 (m, 6H), 1.43-1.84 (m, 3H), 1.87-2.10 (m, 2H), 2.30-2.65 (m, 1H), 3.84-4.23 (m, 1H), 4.27-4.72 (m, 1H), 5.44-5.88 (m, 1H), 6.03-6.45 (m, 1H), 6.58-6.89 (m, 2H), 7.28-7.44 (m, 1H), 7.45-7.72 (m, 1H), 8.13-8.30 (m, 1H), 9.54-10.61 (m, 1H), 12.61-13.44 (m, 1H).

LCMS(ESI): [M+H]⁺ m/z: calcd 411.2; found 411.2; Rt=2.205 min.

### Compound 234:

Preparative: RT (Column: CHIRALPAK IA (250×30 mm, 10 mkm); Mobile Phase:

Hexane:IPA:MeOH:DEA, 70:15:15; Flow Rate: 35 mL/min)=17.377 min.

Analytical: RT (Column: Chiralpak IA (250×4.6 mm, 5 mkm)-1; Mobile Phase: Hexane (0.1% EDA):IPA:MeOH, 70:15:15; Flow Rate: 0.6 mL/min)=16.720 min.

¹H NMR (600 MHz, dmso) δ 0.69-0.92 (m, 3H), 1.05-1.40 (m, 7H), 1.57-2.08 (m, 4H), 2.61-2.92 (m, 1H), 3.52-4.24 (m, 1H), 4.26-4.69 (m, 1H), 5.45-5.88 (m, 1H), 6.03-6.46 (m, 1H), 6.56-6.90 (m, 2H), 7.28-7.45 (m, 1H), 7.45-7.69 (m, 1H), 8.05-8.31 (m, 1H), 9.56-10.68 (m, 1H), 12.66-13.47 (m, 1H).

LCMS(ESI): [M+H]⁺ m/z: calcd 411.2; found 411.2; Rt=2.203 min.

### Example 244. N-(4-Amino-1H-pyrazolo[4,3-c]pyridin-7-yl)-2-oxo-2-[rac-(2R,5S)-2-(2,3-dihydrobenzofuran-4-yl)-5-methyl-1-piperidyl]acetamide (Compound 282)

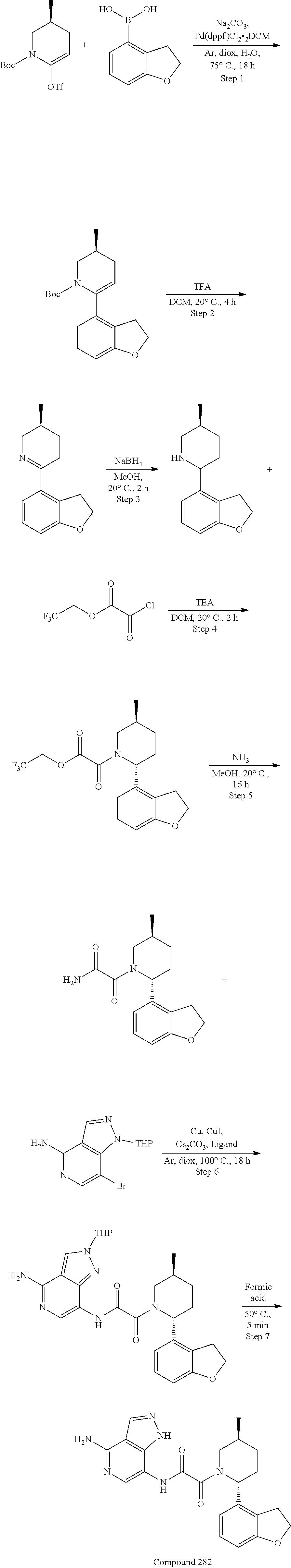

### Step 1: The Synthesis of tert-Butyl rac-(3S)-6-(2,3-dihydrobenzofuran-4-yl)-3-methyl-3,4-dihydro-2H-pyridine-1-carboxylate

Potassium carbonate (2.53 g, 18.30 mmol) was added to the solution of 2,3-dihydrobenzofuran-4-ylboronic acid (1.5 g, 9.15 mmol) and tert-butyl rac-(3S)-3-methyl-6-(trifluoromethylsulfonyloxy)-3,4-dihydro-2H-pyridine-1-carboxylate (3.79 g, 10.98 mmol) in Dioxane (25 mL) and Water (10 mL). Reaction flask was evacuated and refilled with argon 3 times. Then, PdCl₂*dppf*dcm (224.12 mg, 274.45 μmol) was added under stream of argon. Resulting mixture was stirred at 75° C. for 18 hr under inert atmosphere. Then, it was concentrated under reduced pressure and residue was purified by gradient column chromatography (SiO₂, Hex/MTBE), affording tert-butyl rac-(3S)-6-(2,3-dihydrobenzofuran-4-yl)-3-methyl-3,4-dihydro-2H-pyridine-1-carboxylate (1.2 g, 3.80 mmol, 41.59% yield).

LCMS(ESI): [M−tBu]⁺ m/z: calcd 260.2; found 260.0; Rt=1.512 min.

### Step 2: The Synthesis of rac-(3S)-6-(2,3-Dihydrobenzofuran-4-yl)-3-methyl-2,3,4,5-tetrahydropyridine

Trifluoroacetic acid (4.47 g, 39.20 mmol, 3 mL) was added to the solution of tert-butyl rac-(3S)-6-(2,3-dihydrobenzofuran-4-yl)-3-methyl-3,4-dihydro-2H-pyridine-1-carboxylate (1.2 g, 3.80 mmol) in Dichloromethane (10 mL). Resulting mixture was stirred at 20° C. for 4 hr. Then, volatiles were removed under reduced pressure and residue was taken up in water (20 mL). Insoluble tar material was filtered off through a cotton wool plug. Clear filtrate was basified with solid K₂CO₃ to pH≈10 and extracted with DCM (2×15 mL). Combined organic layers were dried over K₂CO₃ and concentrated in vacuo, affording rac-(3S)-6-(2,3-dihydrobenzofuran-4-yl)-3-methyl-2,3,4,5-tetrahydropyridine (0.66 g, 3.07 mmol, 80.58% yield).

LCMS(ESI): [M+H]⁺ m/z: calcd 216.2; found 216.2; Rt=0.615 min.

### Step 3: The Synthesis of rac-(2R,5S)-2-(2,3-Dihydrobenzofuran-4-yl)-5-methyl-piperidine

To the stirred solution of rac-(3S)-6-(2,3-dihydrobenzofuran-4-yl)-3-methyl-2,3,4,5-tetrahydropyridine (0.66 g, 3.07 mmol) in Methanol (25 mL) was added Sodium borohydride (231.96 mg, 6.13 mmol, 215.98 μL) in portions. Resulting mixture was stirred at 20° C. for 2 hr. Then, solvent was removed under reduced pressure and residue was partitioned between water (30 mL) and DCM (30 mL). Organic layer was separated, dried over K₂CO₃ and concentrated in vacuo, affording rac-(2R,5S)-2-(2,3-dihydrobenzofuran-4-yl)-5-methyl-piperidine (0.62 g, 2.85 mmol, 93.07% yield).

LCMS(ESI): [M+H]⁺ m/z: calcd 218.2; found 218.4; Rt=0.658 min.

### Step 4: The Synthesis of 2,2,2-Trifluoroethyl 2-oxo-2-[rac-(2R,5S)-2-(2,3-dihydrobenzofuran-4-yl)-5-methyl-1-piperidyl]acetate

2,2,2-Trifluoroethyl 2-chloro-2-oxo-acetate (706.59 mg, 3.71 mmol) was added dropwise to the solution of rac-(2R,5S)-2-(2,3-dihydrobenzofuran-4-yl)-5-methyl-piperidine (0.62 g, 2.85 mmol) and Triethylamine (866.11 mg, 8.56 mmol, 1.19 mL) in Dichloromethane (20 mL) at 0° C. After addition was complete, cooling bath was removed and resulting mixture was allowed to warm up to 20° C. and stirred for 2 hr. Then, 10% aq. NaHCO₃ solution (20 mL) was added and stirring was continued for 5 min. After that, organic layer was separated, dried over Na₂SO₄ and concentrated under reduced pressure, affording 2,2,2-trifluoroethyl 2-oxo-2-[rac-(2R,5S)-2-(2,3-dihydrobenzofuran-4-yl)-5-methyl-1-piperidyl]acetate (1.1 g, crude).

LCMS(ESI): [M+H]⁺ m/z: calcd 372.2; found 372.2; Rt=1.355 min.

### Step 5: The Synthesis of 2-Oxo-2-[rac-(2R,5S)-2-(2,3-dihydrobenzofuran-4-yl)-5-methyl-1-piperidyl]acetamide

2,2,2-Trifluoroethyl 2-oxo-2-[rac-(2R,5S)-2-(2,3-dihydrobenzofuran-4-yl)-5-methyl-1-piperidyl]acetate (1.1 g, 2.96 mmol) was dissolved in Ammonia (7N in methanol, 15.3% w/w) (11.68 g, 104.98 mmol, 15 mL, 15.3% purity). Resulting reaction mixture was stirred at 20° C. for 16 hr. Then, volatiles were removed under reduced pressure and residue was purified by gradient column chromatography (SiO₂; CHCl₃/ACN), affording 2-oxo-2-[rac-(2R,5S)-2-(2,3-dihydrobenzofuran-4-yl)-5-methyl-1-piperidyl]acetamide (0.5 g, 1.73 mmol, 58.54% yield).

LCMS(ESI): [M+H]⁺ m/z: calcd 289.2; found 289.2; Rt=1.132 min.

### Step 6: The Synthesis of N-(4-Amino-2-tetrahydropyran-2-yl-pyrazolo[4,3-c]pyridin-7-yl)-2-oxo-2-[rac-(2R,5S)-2-(2,3-dihydrobenzofuran-4-yl)-5-methyl-1-piperidyl]acetamide

2-Oxo-2-[rac-(2R,5S)-2-(2,3-dihydrobenzofuran-4-yl)-5-methyl-1-piperidyl]acetamide (150 mg, 520.22 μmol), 7-bromo-2-tetrahydropyran-2-yl-pyrazolo[4,3-c]pyridin-4-amine (177.77 mg, 598.25 μmol), Copper (3.31 mg, 52.02 μmol), Copper (I) iodide (49.54 mg, 260.11 μmol, 8.81 μL), (S,S)—(+)—N,N′-Dimethyl-1,2-cyclohexanediamine (37.00 mg, 260.11 μmol) and Cesium carbonate (254.25 mg, 780.33 μmol) were mixed together in Dioxane (3 mL). Reaction flask was purged with argon and resulting mixture was stirred at 100° C. for 20 hr under inert atmosphere. Then, it was diluted with DCM (10 mL) and filtered. Filtrate was concentrated under reduced pressure, leaving N-(4-amino-2-tetrahydropyran-2-yl-pyrazolo[4,3-c]pyridin-7-yl)-2-oxo-2-[rac-(2R,5S)-2-(2,3-dihydrobenzofuran-4-yl)-5-methyl-1-piperidyl]acetamide (255 mg, 505.37 μmol, 97.15% yield).

LCMS(ESI): [M+H]⁺ m/z: calcd 505.2; found 505.2; Rt=1.018 min.

### Step 7: The Synthesis of N-(4-amino-1H-pyrazolo[4,3-c]pyridin-7-yl)-2-oxo-2-[rac-(2R,5S)-2-(2,3-dihydrobenzofuran-4-yl)-5-methyl-1-piperidyl]acetamide (Compound 282)

N-(4-Amino-2-tetrahydropyran-2-yl-pyrazolo[4,3-c]pyridin-7-yl)-2-oxo-2-[rac-(2R,5S)-2-(2,3-dihydrobenzofuran-4-yl)-5-methyl-1-piperidyl]acetamide (250 mg, 495.46 μmol) was dissolved in Formic acid (2.87 g, 53.01 mmol, 2.35 mL, 85% purity). Resulting mixture was stirred at 50° C. for 5 min. Then, it was subjected to HPLC (10-10-60% 0-1-5 min H₂O/ACN/0.2% FA, flow: 30 mL/min; column: Chromatorex 18 SMB100-5T 100×19 mm 5 um), affording N-(4-amino-1H-pyrazolo[4,3-c]pyridin-7-yl)-2-oxo-2-[rac-(2R,5S)-2-(2,3-dihydrobenzofuran-4-yl)-5-methyl-1-piperidyl]acetamide (40 mg, 95.13 μmol, 19.20% yield).

LCMS(ESI): [M+H]⁺ m/z: calcd 421.2; found 421.2; Rt=2.161 min.

### Example 245. N-(4-Amino-1H-pyrazolo[4,3-c]pyridin-7-yl)-2-oxo-2-[rac-(2R,5S)-2-(2-cyclopropylindazol-6-yl)-5-methyl-1-piperidyl]acetamide (Compound 284)

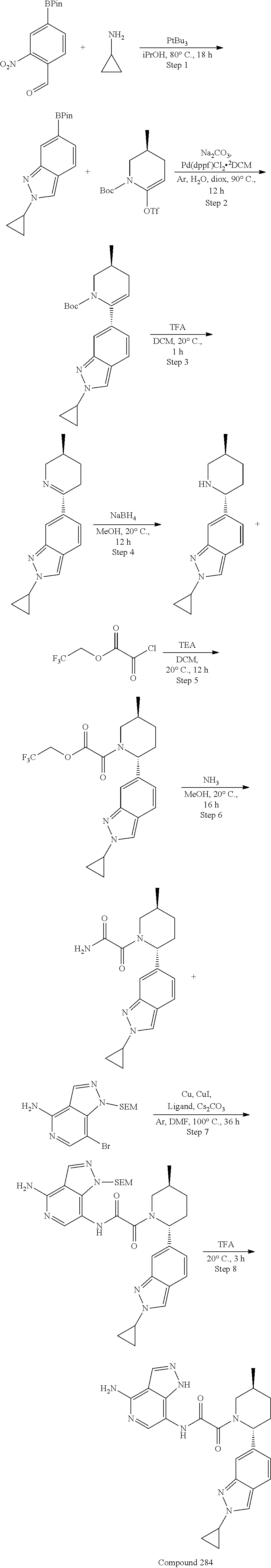

### Step 1: The Synthesis of 2-Cyclopropyl-6-(4,4,5,5-tetramethyl-1,3,2-dioxaborolan-2-yl)indazole

2-Nitro-4-(4,4,5,5-tetramethyl-1,3,2-dioxaborolan-2-yl)benzaldehyde (5 g, 18.05 mmol) and cyclopropanamine (2.06 g, 36.09 mmol, 2.50 mL) were dissolved in i-PrOH (50 mL) and the resulting solution was heated at 80° C. for 2 hr in a sealed tube. The reaction mixture was cooled to RT and Tri-n-butylphosphine, 95% (10.95 g, 54.14 mmol, 13.36 mL) was added, then the resulting mixture was heated at 80° C. overnight in a sealed tube. The reaction mixture was concentrated on vacuo. The crude product was purified by FCC (MTBE in hexanes from 16% to 50%). The reaction is successful. 2-Cyclopropyl-6-(4,4,5,5-tetramethyl-1,3,2-dioxaborolan-2-yl)indazole (1.6 g, 5.63 mmol, 31.20% yield) was obtained as a brown gum.

LCMS(ESI): [M+H]⁺ m/z: calcd 285.2; found 285.2; Rt=1.354 min.

### Step 2: The Synthesis of tert-Butyl rac-(3S)-6-(2-cyclopropylindazol-6-yl)-3-methyl-3,4-dihydro-2H-pyridine-1-carboxylate

2-Cyclopropyl-6-(4,4,5,5-tetramethyl-1,3,2-dioxaborolan-2-yl)indazole (1.6 g, 5.63 mmol), tert-butyl rac-(3S)-3-methyl-6-(trifluoromethylsulfonyloxy)-3,4-dihydro-2H-pyridine-1-carboxylate (2.14 g, 6.19 mmol), Sodium carbonate (1.19 g, 11.26 mmol, 471.40 μL) and Pd(dppf)Cl₂·DCM (229.83 mg, 281.53 μmol) were mixed in dioxane (10 mL)+water (3 mL) under argon, and then stirred overnight at 90° C. for 12 hr in vial. The solid was filtered off, the filtrate was diluted with EA, the organic layer was separated, and the aqueous layer was extracted with additional EA, the combined organic layer was washed with brine, dried over Na₂SO₄, filtered and concentrated on vacuo. The obtained product was used in the next step without further purification. The reaction was successful. tert-Butyl rac-(3S)-6-(2-cyclopropylindazol-6-yl)-3-methyl-3,4-dihydro-2H-pyridine-1-carboxylate (3.11 g, crude) was obtained as a red gum.

LCMS(ESI): [M+H]⁺ m/z: calcd 354.2; found 354.2; Rt=1.297 min.

### Step 3: The Synthesis of 2-Cyclopropyl-6-[rac-(3S)-3-methyl-2,3,4,5-tetrahydropyridin-6-yl]indazole

tert-Butyl rac-(3S)-6-(2-cyclopropylindazol-6-yl)-3-methyl-3,4-dihydro-2H-pyridine-1-carboxylate (3.11 g, 8.80 mmol) was dissolved in a mixture of TFA (15 mL) and DCM (15 mL). The resulting clear solution was stirred for 1 hr at 20° C. The reaction mixture was concentrated on vacuo. The obtained residue was dissolved in water and extracted with MTBE (3 times), after that the aqueous layer was basified with NaOH and extracted with DCM (3 times), combined DCM layers was dried over Na₂SO₄, filtered and evaporated. The reaction is successful. 2-Cyclopropyl-6-[rac-(3S)-3-methyl-2,3,4,5-tetrahydropyridin-6-yl]indazole (0.7 g, 2.76 mmol, 31.40% yield) was obtained as a brown solid.

LCMS(ESI): [M+H]⁺ m/z: calcd 254.2; found 254.2; Rt=0.694 min.

### Step 4: The Synthesis of 2-Cyclopropyl-6-[rac-(2R,5S)-5-methyl-2-piperidyl]indazole

To a stirring suspension of 2-cyclopropyl-6-[rac-(3S)-3-methyl-2,3,4,5-tetrahydropyridin-6-yl]indazole (0.7 g, 2.76 mmol) in MeOH (15 mL) was added Sodium Borohydride (209.07 mg, 5.53 mmol, 194.66 μL) in portions. Resulting mixture was stirred at 20° C. for 12 hr. The solvent was removed under reduced pressure and residue was partitioned between water and DCM. The organic layer was separated, dried over Na₂SO₄ and concentrated in vacuo. The obtained product was used in the next step without further purification. The reaction is successful. 2-Cyclopropyl-6-[rac-(2R,5S)-5-methyl-2-piperidyl]indazole (0.35 g, 1.37 mmol, 49.61% yield) was obtained as a brown gum.

LCMS(ESI): [M+H]⁺ m/z: calcd 256.4; found 256.4; Rt=0.625 min.

### Step 5: The Synthesis of 2,2,2-Trifluoroethyl 2-oxo-2-[rac-(2R,5S)-2-(2-cyclopropylindazol-6-yl)-5-methyl-1-piperidyl]acetate

2,2,2-Trifluoroethyl 2-chloro-2-oxo-acetate (287.22 mg, 1.51 mmol, 261.11 μL) was added dropwise to a solution of 2-cyclopropyl-6-[rac-(2R,5S)-5-methyl-2-piperidyl]indazole (0.35 g, 1.37 mmol) and TEA (159.50 mg, 1.58 mmol, 219.69 μL) in DCM (10.74 mL) at 0° C. After addition was complete, cooling bath was removed and resulting mixture was allowed to warm up to 20° C. and stirred for 12 hr. The reaction mixture was washed with Na₂CO₃, brine, dried over Na₂SO₄ and concentrated under reduced pressure. The obtained product was used in the next step without further purification. The reaction is successful. 2,2,2-Trifluoroethyl 2-oxo-2-[rac-(2R,5S)-2-(2-cyclopropylindazol-6-yl)-5-methyl-1-piperidyl]acetate (0.65 g, crude) was obtained as a brown gum.

LCMS(ESI): [M+H]⁺ m/z: calcd 410.1; found 410.1; Rt=1.481 min.

### Step 6: The Synthesis of 2-Oxo-2-[rac-(2R,5S)-2-(2-cyclopropylindazol-6-yl)-5-methyl-1-piperidyl]acetamide

2,2,2-Trifluoroethyl 2-oxo-2-[rac-(2R,5S)-2-(2-cyclopropylindazol-6-yl)-5-methyl-1-piperidyl]acetate (0.6 g, 1.47 mmol) was dissolved in MeOH(NH₃) (15 mL) as stirred overnight at 20° C. The reaction mixture was evaporated to dryness. The obtained product was used in the next step without further purification. The reaction is successful. 2-Oxo-2-[rac-(2R,5S)-2-(2-cyclopropylindazol-6-yl)-5-methyl-1-piperidyl]acetamide (0.3 g, 919.14 μmol, 62.72% yield) was obtained as a brown gum.

LCMS(ESI): [M+H]⁺ m/z: calcd 327.2; found 327.2; Rt=1.037 min.

### Step 7: The Synthesis of N-[4-Amino-1-(2-trimethylsilylethoxymethyl)pyrazolo[4,3-c]pyridin-7-yl]-2-oxo-2-[rac-(2R,5S)-2-(2-cyclopropylindazol-6-yl)-5-methyl-1-piperidyl]acetamide

2-Oxo-2-[rac-(2R,5S)-2-(2-cyclopropylindazol-6-yl)-5-methyl-1-piperidyl]acetamide (0.3 g, 919.14 μmol), 7-bromo-1-(2-trimethylsilylethoxymethyl)pyrazolo[4,3-c]pyridin-4-amine (378.64 mg, 1.10 mmol), Copper (I) iodide (35.01 mg, 183.83 μmol, 6.23 μL), Cesium carbonate (598.95 mg, 1.84 mmol) and (1R,2R)—N1,N2-dimethylcyclohexane-1,2-diamine (156.89 mg, 1.10 mmol) were mixed in DMF (5 mL) under argon, and then stirred overnight at 100° C. for 36 hr in vial. The reaction mixture was submitted to HPLC (2-10 min; 30-60% water-MeCN+FA; flow: 30 mL/min; loading pump 4 mL/min MeCN+FA; column SunFire 19*100 mm). The reaction was successful. N-[4-Amino-1-(2-trimethylsilylethoxymethyl)pyrazolo[4,3-c]pyridin-7-yl]-2-oxo-2-[rac-(2R,5S)-2-(2-cyclopropylindazol-6-yl)-5-methyl-1-piperidyl]acetamide (0.052 g, 88.32 μmol, 9.61% yield) was obtained as a brown solid.

LCMS(ESI): [M+H]⁺ m/z: calcd 589.2; found 589.2; Rt=1.344 min.

### Step 8: The Synthesis of N-(4-Amino-1H-pyrazolo[4,3-c]pyridin-7-yl)-2-oxo-2-[rac-(2R,5S)-2-(2-cyclopropylindazol-6-yl)-5-methyl-1-piperidyl]acetamide (Compound 284)

N-[4-Amino-1-(2-trimethylsilylethoxymethyl)pyrazolo[4,3-c]pyridin-7-yl]-2-oxo-2-[rac-(2R,5S)-2-(2-cyclopropylindazol-6-yl)-5-methyl-1-piperidyl]acetamide (0.052 g, 88.32 μmol) was dissolved in TFA (302.11 mg, 2.65 mmol, 204.13 μL) and stirred at 20° C. for 3 hr. The reaction mixture was submitted to HPLC (2-10 min 0-90% water-methanol+FA; flow: 30 mL/min; loading pump 4 mL/min methanol+FA: column SunFire 19*100 mm). The reaction is successful. N-(4-Amino-1H-pyrazolo[4,3-c]pyridin-7-yl)-2-oxo-2-[rac-(2R,5S)-2-(2-cyclopropylindazol-6-yl)-5-methyl-1-piperidyl]acetamide (0.014 g, 24.45 μmol, 27.69% yield, CF₃COOH) was obtained as a light-yellow solid.

¹H NMR (600 MHz, dmso) δ 1.01-1.07 (m, 3H), 1.07-1.12 (m, 2H), 1.22-1.28 (m, 2H), 1.28-1.43 (m, 1H), 1.69-1.84 (m, 1H), 1.84-1.97 (m, 1H), 2.02-2.26 (m, 1H), 2.26-2.36 (m, 1H), 2.78-3.03 (m, 1H), 3.72-4.12 (m, 2H), 5.34-5.73 (m, 1H), 6.91-7.23 (m, 2H), 7.23-7.61 (m, 2H), 7.61-7.70 (m, 1H), 7.70-7.86 (m, 1H), 8.16-8.46 (m, 2H), 10.49-10.86 (m, 1H), 12.50-13.25 (m, 1H).

LCMS(ESI): [M+H]⁺ m/z: calcd 459.2; found 459.2; Rt=0.685 min.

### INTERMEDIATES

### Intermediate 1. N,N-dimethyl-2-(5-((2R,5S)-5-methylpiperidin-2-yl)benzo[d]thiazol-2-yl)ethanamine

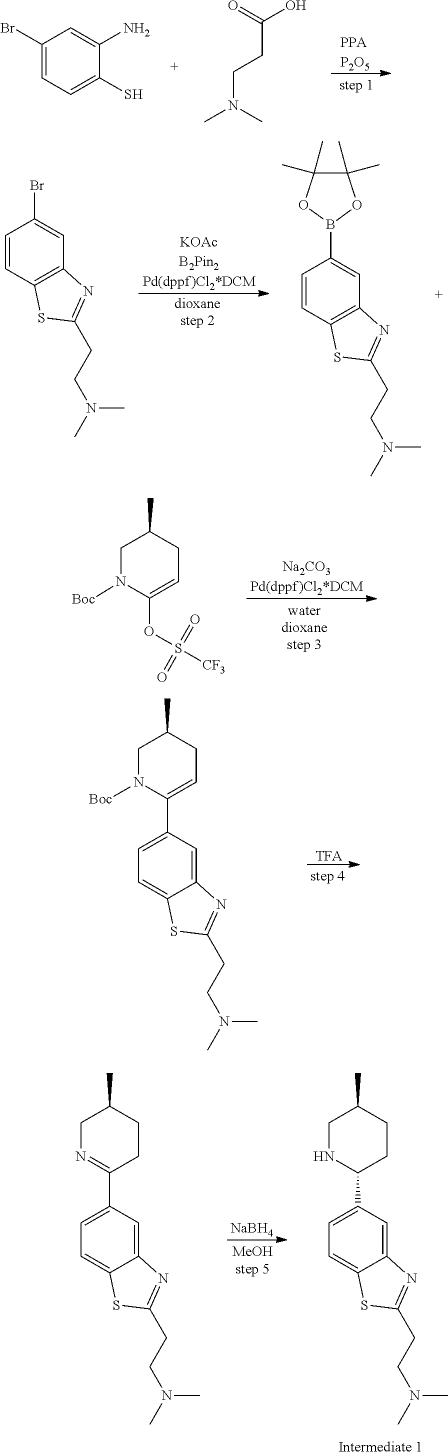

### Step 1: Synthesis of 2-(5-bromobenzo[d]thiazol-2-yl)-N,N-dimethylethanamine

Prepared by general procedure scheme S2 step 1A. Yield: 6.9 g (98.75%).

LCMS(ESI): [M]⁺ m/z: calcd 285.2; found 286.2; Rt=0.790 min.

### Step 2: Synthesis of N,N-dimethyl-2-(5-(4,4,5,5-tetramethyl-1,3,2-dioxaborolan-2-yl)benzo[d]thiazol-2-yl)ethanamine

Prepared by general procedure scheme S2 step 2. Yield: 16.6 g of crude.

LCMS(ESI): [M]⁺ m/z: calcd 332.2; found 333.2; Rt=0.920 min.

### Step 3: Synthesis of (S)-tert-butyl 6-(2-(2-(dimethylamino)ethyl)benzo[d]thiazol-5-yl)-3-methyl-3,4-dihydropyridine-1(2H)-carboxylate

Prepared by general procedure scheme S2 step 3. Yield: 25 g of crude.

LCMS(ESI): [M]⁺ m/z: calcd 401.2; found 402.2; Rt=1.110 min.

### Step 4: Synthesis of (S)—N,N-dimethyl-2-(5-(5-methyl-3,4,5,6-tetrahydropyridin-2-yl)benzo[d]thiazol-2-yl)ethanamine

Prepared by general procedure scheme S2 step 4. Yield: 4.8 g of crude.

LCMS(ESI): [M]⁺ m/z: calcd 301.2; found 302.2; Rt=0.637 min.

### Step 5: Synthesis of N,N-dimethyl-2-(5-((2R,5S)-5-methylpiperidin-2-yl)benzo[d]thiazol-2-yl)ethanamine

Prepared by general procedure scheme S2 step 5. Yield: 1.4 g of crude.

LCMS(ESI): [M]⁺ m/z: calcd 303.2; found 304.2; Rt=0.503 min.

### Intermediate 2. 5-((2R,5S)-5-methylpiperidin-2-yl)-2-(1-methylpiperidin-4-yl)benzo[d]thiazole

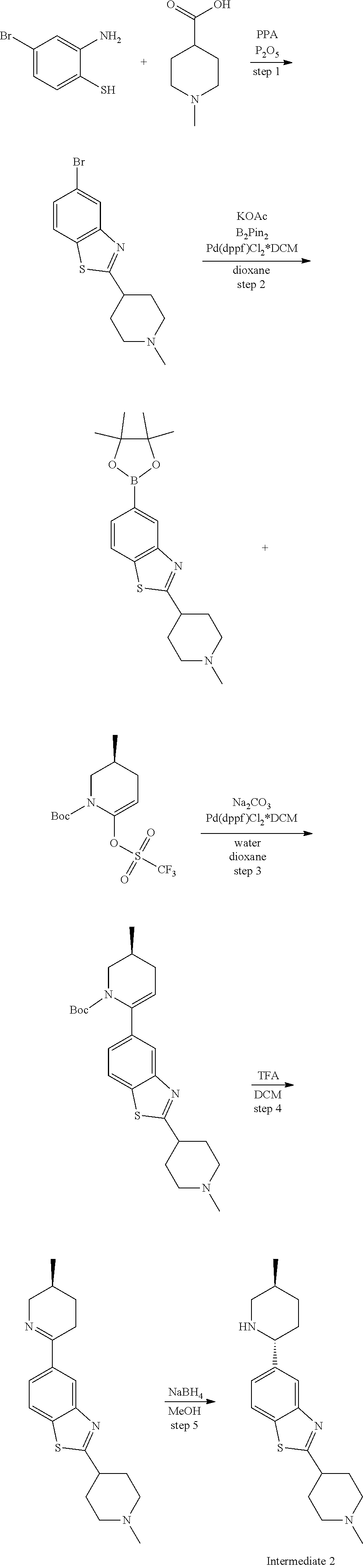

### Step 1: Synthesis of 5-bromo-2-(1-methylpiperidin-4-yl)benzo[d]thiazole

Prepared by general procedure scheme S2 step 1A. Yield: 14 g (92.88%).

LCMS(ESI): [M]⁺ m/z: calcd 311.2; found 312.2; Rt=0.742 min.

### Step 2: Synthesis of 2-(1-methylpiperidin-4-yl)-5-(4,4,5,5-tetramethyl-1,3,2-dioxaborolan-2-yl)benzo[d]thiazole

Prepared by general procedure scheme S2 step 2. Yield: 8.5 g (92.29%).

LCMS(ESI): [M]⁺ m/z: calcd 358.2; found 359.2; Rt=0.978 min.

### Step 3: Synthesis of (S)-tert-butyl 3-methyl-6-(2-(1-methylpiperidin-4-yl)benzo[d]thiazol-5-yl)-3,4-dihydropyridine-1(2H)-carboxylate

Prepared by general procedure scheme S2 step 3. Yield: 15 g of crude.

LCMS(ESI): [M]⁺ m/z: calcd 427.2; found 428.2; Rt=1.274 min.

### Step 4: Synthesis of (S)-5-(5-methyl-3,4,5,6-tetrahydropyridin-2-yl)-2-(1-methylpiperidin-4-yl)benzo[d]thiazole

Prepared by general procedure scheme S2 step 4. Yield: 6 g (94.96%).

LCMS(ESI): [M]⁺ m/z: calcd 327.2; found 328.2; Rt=0.670 min.

### Step 5: Synthesis of 5-((2R,5S)-5-methylpiperidin-2-yl)-2-(1-methylpiperidin-4-yl)benzo[d]thiazole

Prepared by general procedure scheme S2 step 5. Yield: 4 g (66.26%).

LCMS(ESI): [M]⁺ m/z: calcd 329.2; found 330.2; Rt=0.703 min.

### Intermediate 3. 2-(1-cyclopropylpiperidin-4-yl)-5-((2R,5S)-5-methylpiperidin-2-yl)benzo[d]thiazole

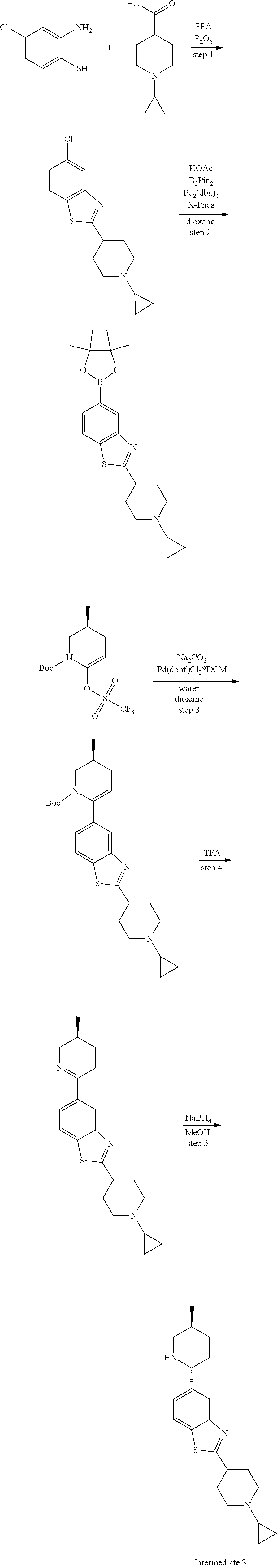

### Step 1: Synthesis of 5-chloro-2-(1-cyclopropylpiperidin-4-yl)benzo[d]thiazole

Prepared by general procedure scheme S2 step 1A. Yield: 8 g (90.63%).

LCMS(ESI): [M]⁺ m/z: calcd 292.2; found 293.2; Rt=0.848 min.

### Step 2: Synthesis of 2-(1-cyclopropylpiperidin-4-yl)-5-(4,4,5,5-tetramethyl-1,3,2-dioxaborolan-2-yl)benzo[d]thiazole

5-Chloro-2-(1-cyclopropyl-4-piperidyl)-1,3-benzothiazole (4 g, 13.66 mmol), 4,4,5,5-tetramethyl-2-(4,4,5,5-tetramethyl-1,3,2-dioxaborolan-2-yl)-1,3,2-dioxaborolane (3.82 g, 15.03 mmol) and potassium acetate (2.68 g, 27.32 mmol, 1.71 mL) were mixed in dioxane (70 mL). The resulting mixture was evacuated and then backfilled with argon, this operation was repeated three times, then tris(dibenzylideneacetone)dipalladium(0) (625.43 mg, 683.00 μmol) was added under argon. The reaction mixture was stirred under argon at 90° C. for 14 hr, then cooled and concentrated under reduce pressure. The residue was purified by column chromatography to afford 2-(1-cyclopropyl-4-piperidyl)-5-(4,4,5,5-tetramethyl-1,3,2-dioxaborolan-2-yl)-1,3-benzothiazole (2.4 g, 6.24 mmol, 45.71% yield). LCMS(ESI): [M]⁺.

m/z: calcd 384.2; found 385.2; Rt=1.026 min.

### Step 3: Synthesis of (S)-tert-butyl 6-(2-(1-cyclopropylpiperidin-4-yl)benzo[d]thiazol-5-yl)-3-methyl-3,4-dihydropyridine-1(2H)-carboxylate

Prepared by general procedure scheme S2 step 3. Yield: 0.6 g (29.90%).

CC conditions: The crude product was purified by silica gel with Hexane/EtOAc as an eluent mixture.

LCMS(ESI): [M]⁺ m/z: calcd 453.2; found 454.2; Rt=1.372 min.

### Step 4: Synthesis of (S)-2-(1-cyclopropylpiperidin-4-yl)-5-(5-methyl-3,4,5,6-tetrahydropyridin-2-yl)benzo[d]thiazole

Prepared by general procedure scheme S2 step 4. Yield: 0.3 g of crude.

LCMS(ESI): [M]⁺ m/z: calcd 353.2; found 354.2; Rt=0.719 min.

### Step 5: Synthesis of 2-(1-cyclopropylpiperidin-4-yl)-5-((2R,5S)-5-methylpiperidin-2-yl)benzo[d]thiazole

Prepared by general procedure scheme S2 step 5. Yield: 0.28 g of crude.

LCMS(ESI): [M]⁺ m/z: calcd 355.2; found 356.2; Rt=0.751 min.

### Intermediate 4, N,N-dimethyl-1-(3-(5-((2R,5S)-5-methylpiperidin-2-yl)benzo[d]thiazol-2-yl)oxetan-3-yl)methanamine

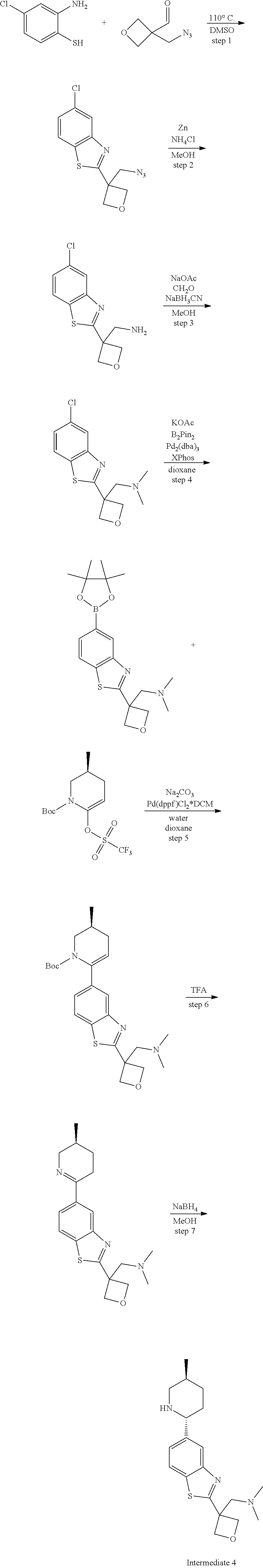

### Step 1: Synthesis of 2-(3-(azidomethyl)oxetan-3-yl)-5-chlorobenzo[d]thiazole

Prepared by general procedure scheme S2 step 1B. Yield: 11 g of crude.

LCMS(ESI): [M]⁺ m/z: calcd 280.2; found 281.2; Rt=3.578 min.

### Step 2: Synthesis of (3-(5-chlorobenzo[d]thiazol-2-yl)oxetan-3-yl)methanamine

Crude MTBE solution of 2-[3-(azidomethyl)oxetan-3-yl]-5-chloro-1,3-benzothiazole (11 g, 39.18 mmol) (approximately 50 ml) was diluted with methanol (100 mL) and ammonium chloride (12.58 g, 235.10 mmol, 8.22 mL) was added. Zinc (7.69 g, 117.55 mmol) dust was added portion wise with stirring at 25° C. to the above mixture. The reaction mixture was stirred at 25° C. for 12 hr, and then filtered. The filter cake was washed with MeOH (2*25 ml) and discarded. The combined filtrate was concentrated in vacuum. The residue was diluted with MTBE (100 ml) and extracted with 5% aqueous sodium hydrogen sulphate solution (100 ml). The resulting aqueous solution of amine hydrogen sulphate was then basified to pH 11 with 10% aqueous sodium hydroxide solution and extracted with DCM (2*50 ml). The combined organic extracts were dried over sodium sulphate and concentrated in vacuum to afford crude [3-(5-chloro-1,3-benzothiazol-2-yl)oxetan-3-yl]methanamine (1.2 g, 4.71 mmol, 12.02% yield) as light-yellow gum, which was used directly in the next step. LCMS(ESI): [M]⁺ m/z: calcd 254.2; found 255.2; Rt=1.985 min.

### Step 3: Synthesis of 1-(3-(5-chlorobenzo[d]thiazol-2-yl)oxetan-3-yl)-N,N-dimethylmethanamine

Formaldehyde, 37% w/w aq. soln., stab. with 7-8% MeOH (946.16 mg, 11.66 mmol, 873.64 μL, 37% purity) and acetic acid (518.63 mg, 8.64 mmol, 494.41 μL) were added to a stirred solution of [3-(5-chloro-1,3-benzothiazol-2-yl)oxetan-3-yl]methanamine (1.1 g, 4.32 mmol) in MeOH (30.49 mL) at 25° C. The resulting mixture was stirred at 25° C. for 1 hr, then sodium cyanoborohydride (542.73 mg, 8.64 mmol) was added in one portion at 25° C. (foaming!). The reaction mixture was stirred at 25° C. for 12 hr, and then concentrated in vacuum. The residue was diluted with 10% aqueous sodium hydroxide solution (20 ml) and extracted with DCM (2*20 ml). The combined organic extracts were dried over sodium sulphate and concentrated in vacuum to afford crude 1-[3-(5-chloro-1,3-benzothiazol-2-yl)oxetan-3-yl]-N,N-dimethyl-methanamine (1.3 g, crude) as light-yellow gum, which was used directly in the next step. LCMS(ESI): [M]⁺ m/z: calcd 282.2; found 283.2; Rt=1.530 min.

### Step 4: Synthesis of N,N-dimethyl-1-(3-(5-(4,4,5,5-tetramethyl-1,3,2-dioxaborolan-2-yl)benzo[d]thiazol-2-yl)oxetan-3-yl)methanamine)

A mixture of 1-[3-(5-chloro-1,3-benzothiazol-2-yl)oxetan-3-yl]-N,N-dimethyl-methanamine (1.4 g, 4.95 mmol), 4,4,5,5-tetramethyl-2-(4,4,5,5-tetramethyl-1,3,2-dioxaborolan-2-yl)-1,3,2-dioxaborolane (1.38 g, 5.45 mmol) and potassium acetate (971.72 mg, 9.90 mmol, 618.93 μL) in dioxane (35 mL) was evacuated and then backfilled with argon. This operation was repeated two times, then tris(1,5-diphenylpenta-1,4-dien-3-one) dipalladium(226.67 mg, 247.53 μmol) and dicyclohexyl[2′,4′,6′-tris(propan-2-yl)-[1,1′-biphenyl]-2-yl]phosphane (472.02 mg, 990.14 μmol) were added under argon, and the reaction mixture was stirred at 95° C. for 18 hr. The reaction mixture was cooled down and filtered. The filter cake was washed with dioxane (2*10 ml) and discarded. The combined filtrate was concentrated in vacuum. The residue was diluted with DCM (30 ml) and extracted with a solution of sodium hydrogen sulphate (1.19 g, 9.90 mmol) in water (20 ml) (repeated two times). The combined aqueous layer was basified to pH 10 with 10% aqueous sodium hydroxide solution and back-extracted with DCM (3*25 ml). Evaporation of both DCM extracts does not showed desired product. It seemed like boronic ester hydrolyzed in aqueous media and remained in aqueous phase as boronic acid. The combined water layer (approximately 100 ml) contained N,N-dimethyl-1-[3-[5-(4,4,5,5-tetramethyl-1,3,2-dioxaborolan-2-yl)-1,3-benzothiazol-2-yl]oxetan-3-yl]methanamine (1.85 g, 4.94 mmol, 100.00% yield) (theoretical amount of product, most likely in boronic acid form) was used directly in the next step.

LCMS(ESI): [M]⁺ m/z: calcd 374.2; found 375.2; Rt=2.586 min.

### Step 5: Synthesis of (S)-tert-butyl 6-(2-(3-((dimethylamino)methyl)oxetan-3-yl)benzo[d]thiazol-5-yl)-3-methyl-3,4-dihydropyridine-1(2H)-carboxylate

Prepared by general procedure scheme S2 step 3. Yield: 1.3 g of crude.

LCMS(ESI): [M]⁺ m/z: calcd 443.2; found 444.2; Rt=1.279 min.

### Step 6: Synthesis of (S)—N,N-dimethyl-1-(3-(5-(5-methyl-3,4,5,6-tetrahydropyridin-2-yl)benzo[d]thiazol-2-yl)oxetan-3-yl)methanamine

Prepared by general procedure scheme S2 step 4. Yield: 450 mg of crude.

LCMS(ESI): [M]⁺ m/z: calcd 343.2; found 344.2; Rt=0.624 min.

### Step 7: Synthesis of N,N-dimethyl-1-(3-(5-((2R,5S)-5-methylpiperidin-2-yl)benzo[d]thiazol-2-yl)oxetan-3-yl)methanamine

Prepared by general procedure scheme S2 step 5. Yield: 330 mg (72.91%).

LCMS(ESI): [M]⁺ m/z: calcd 345.2; found 346.2; Rt=0.574 min.

### Intermediate 5. N,N-dimethyl-1-(1-(5-((2R,5S)-5-methylpiperidin-2-yl)benzo[d]thiazol-2-yl)cyclopropyl)methanamine

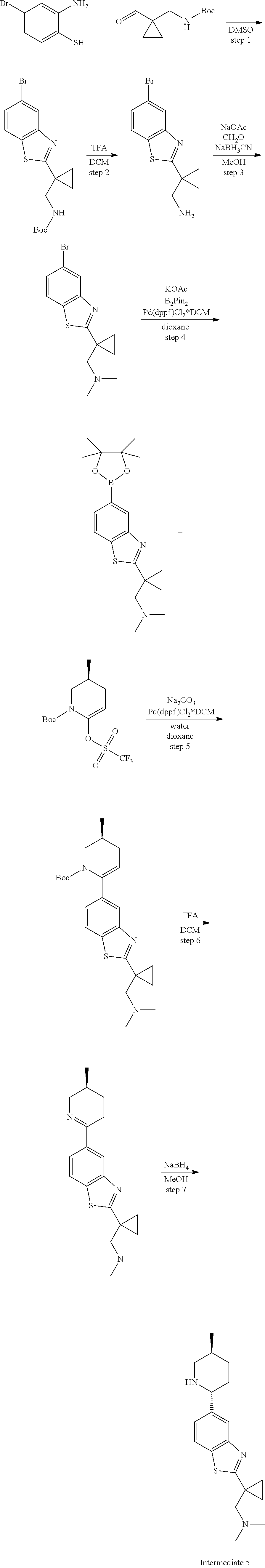

### Step 1: Synthesis of tert-butyl ((1-(5-bromobenzo[d]thiazol-2-yl)cyclopropyl)methyl)carbamate

Prepared by general procedure scheme S2 step 1B. Yield: 5.2 g (92.29%).

LCMS(ESI): [M]⁺ m/z: calcd 383.2; found 384.2; Rt=1.648 min.

### Step 2: Synthesis of (1-(5-bromobenzo[d]thiazol-2-yl)cyclopropyl)methanamine

TFA (22.20 g, 194.70 mmol, 15 mL) was added in one portion to a stirred solution of tert-butyl N-[[1-(5-bromo-1,3-benzothiazol-2-yl)cyclopropyl]methyl]carbamate (2.7 g, 7.04 mmol) in DCM (30 mL) at 25° C. The resulting solution was stirred at 25° C. for 0.5 hr, and then concentrated in vacuum. The residue was diluted with ice cold water (50 ml) and basified to pH 11 with 10% aqueous sodium hydroxide solution. The resulting mixture was extracted with DCM (2*50 ml). The combined organic extracts were dried over sodium sulfate and concentrated in vacuum to afford crude [1-(5-bromo-1,3-benzothiazol-2-yl)cyclopropyl]methanamine (1.9 g, 6.71 mmol, 95.25% yield) as red gum, which was used directly in the next step. LCMS(ESI): [M]⁺ m/z: calcd 383.2; found 384.2; Rt=0.862 min.

### Step 3: Synthesis of 1-(1-(5-bromobenzo[d]thiazol-2-yl)cyclopropyl)-N,N-dimethylmethanamine

Formaldehyde, 37% w/w aq. soln., stab. with 7-8% MeOH (1.6 g, 19.71 mmol, 1.48 mL, 37% purity) and acetic acid (805.79 mg, 13.42 mmol, 768.15 μL) were added to a stirred solution of [1-(5-bromo-1,3-benzothiazol-2-yl)cyclopropyl]methanamine (1.9 g, 6.71 mmol) in MeOH (60 mL) at 25° C. The resulting mixture was stirred at 25° C. for 1 hr, then sodium cyan borohydride (843.23 mg, 13.42 mmol) was added in one portion at 25° C. (foaming!). The reaction mixture was stirred at 25° C. for 18 hr, and then concentrated in vacuum. The residue was diluted with 10% aqueous sodium hydroxide solution (40 ml) and extracted with DCM (2*30 ml). The combined organic extracts were dried over sodium sulphate and concentrated in vacuum to afford crude 1-[1-(5-bromo-1,3-benzothiazol-2-yl)cyclopropyl]-N,N-dimethyl-methanamine (1.9 g, 6.10 mmol, 90.99% yield) as light-brown gum, which was used directly in the next step. LCMS(ESI): [M]⁺ m/z: calcd 311.2; found 312.2; Rt=2.196 min.

### Step 4: Synthesis of N,N-dimethyl-1-(1-(5-(4,4,5,5-tetramethyl-1,3,2-dioxaborolan-2-yl)benzo[d]thiazol-2-yl)cyclopropyl)methanamine

Prepared by general procedure scheme S2 step 2. Yield: 2.19 g of crude.

LCMS(ESI): [M]⁺ m/z: calcd 358.2; found 359.2; Rt=3.096 min.

### Step 5: Synthesis of (S)-tert-butyl 6-(2-(1-((dimethylamino)methyl)cyclopropyl)benzo[d]thiazol-5-yl)-3-methyl-3,4-dihydropyridine-1(2H)-carboxylate

Prepared by general procedure scheme S2 step 3. Yield: 3.9 g of crude.

LCMS(ESI): [M]⁺ m/z: calcd 427.2; found 428.2; Rt=1.315 min.

### Step 6: Synthesis of (S)—N,N-dimethyl-1-(1-(5-(5-methyl-3,4,5,6-tetrahydropyridin-2-yl)benzo[d]thiazol-2-yl)cyclopropyl)methanamine

Prepared by general procedure scheme S2 step 4. Yield: 0.85 g of crude.

LCMS(ESI): [M]⁺ m/z: calcd 327.2; found 328.2; Rt=0.616 min.

### Step 7: Synthesis of N,N-dimethyl-1-(1-(5-((2R,5S)-5-methylpiperidin-2-yl)benzo[d]thiazol-2-yl)cyclopropyl)methanamine

Prepared by general procedure scheme S2 step 5. Yield: 0.67 g (78.34%).

LCMS(ESI): [M]⁺ m/z: calcd 329.2; found 330.2; Rt=0.685 min.

### Intermediate 6. 2-(1-methylazetidin-3-yl)-5-((2R,5S)-5-methylpiperidin-2-yl)benzo[d]thiazole

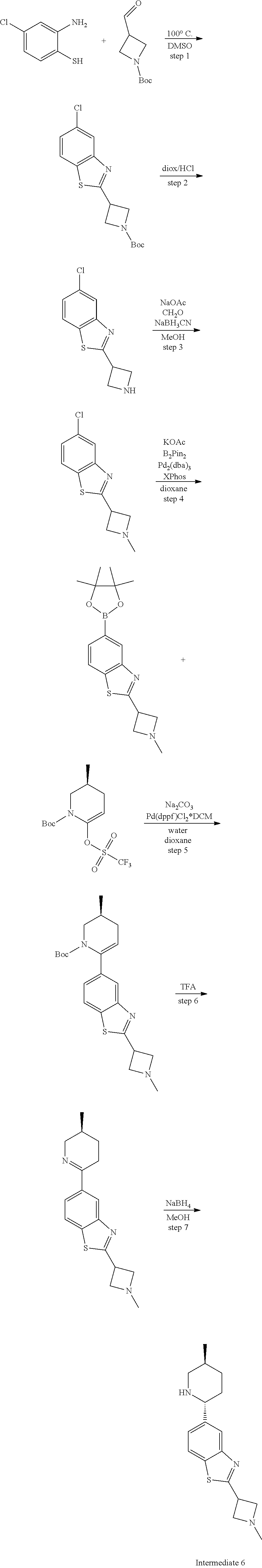

### Step 1: Synthesis of tert-butyl 3-(5-chlorobenzo[d]thiazol-2-yl)azetidine-1-carboxylate

Prepared by general procedure scheme S2 step 1B. Yield: 1.5 g (14.74%).

CC conditions: The crude product was purified by silica gel with hexane/MTBE as an eluent mixture.

LCMS(ESI): [M]⁺ m/z: calcd 324.2; found 325.2; Rt=1.591 min.

### Step 2: Synthesis of 2-(azetidin-3-yl)-5-chlorobenzo[d]thiazole

tert-Butyl 3-(5-chloro-1,3-benzothiazol-2-yl)azetidine-1-carboxylate (2 g, 6.16 mmol) was treated with hydrogen chloride solution 4.0M in dioxane (10.67 g, 292.55 mmol, 13.33 mL). The resulting mixture was stirred at 25° C. for 12 hr. Precipitate was filtered and additionally washed with MTBE. Then dried in vacuum to give 2-(azetidin-3-yl)-5-chloro-1,3-benzothiazole (2 g, crude, HCl). LCMS(ESI): [M]⁺ m/z: calcd 224.2; found 225.2; Rt=0.570 min.

### Step 3: Synthesis of 5-chloro-2-(1-methylazetidin-3-yl)benzo[d]thiazole

To the stirred solution of 2-(azetidin-3-yl)-5-chloro-1,3-benzothiazole (3 g, 11.49 mmol, HCl) in MeOH (24.06 mL) formaldehyde, 37% w/w aq. soln., stab. with 7-8% MeOH (1.40 g, 17.23 mmol, 1.29 mL, 37% purity) and sodium acetate, anhydrous (2.36 g, 28.72 mmol, 1.54 mL) were added. The resulting mixture was stirred for 2 hr at 25° C. Then Sodium cyan borohydride (1.44 g, 22.97 mmol) was added portion wise. The resulting mixture was stirred at 25° C. for 12 hr. Methanol was evaporated. The residue was diluted with water (50 ml) and extracted with DCM (3*25 ml). Combined organic layers were dried over Na₂SO₄. DCM was evaporated in vacuum to give 5-chloro-2-(1-methylazetidin-3-yl)-1,3-benzothiazole (2.3 g, 9.63 mmol, 83.87% yield). LCMS(ESI): [M]⁺ m/z: calcd 238.2; found 239.2; Rt=1.608 min.

### Step 4: Synthesis of 2-(1-methylazetidin-3-yl)-5-(4,4,5,5-tetramethyl-1,3,2-dioxaborolan-2-yl)benzo[d]thiazole

5-Chloro-2-(1-methylazetidin-3-yl)-1,3-benzothiazole (2.3 g, 9.63 mmol), 4,4,5,5-tetramethyl-2-(4,4,5,5-tetramethyl-1,3,2-dioxaborolan-2-yl)-1,3,2-dioxaborolane (2.69 g, 10.60 mmol) and potassium acetate (1.89 g, 19.27 mmol, 1.20 mL) were mixed in dioxane (40 mL). The resulting mixture was evacuated and then backfilled with argon, this operation was repeated three times, then tris(dibenzylideneacetone)dipalladium(0) (441.11 mg, 481.70 μmol) was added under argon. The reaction mixture was stirred under argon at 90° C. for 14 hr, then cooled and concentrated under reduce pressure. The residue was purified by column chromatography to afford 2-(1-methylazetidin-3-yl)-5-(4,4,5,5-tetramethyl-1,3,2-dioxaborolan-2-yl)-1,3-benzothiazole (0.6 g, 1.82 mmol, 18.86% yield). LCMS(ESI): [M]⁺m/z: calcd 330.2; found 331.2; Rt=0.942 min.

### Step 5: Synthesis of (S)-tert-butyl 3-methyl-6-(2-(1-methylazetidin-3-yl)benzo[d]thiazol-5-yl)-3,4-dihydropyridine-1(2H)-carboxylate

Prepared by general procedure scheme S2 step 3. Yield: 0.8 g of crude

LCMS(ESI): [M]⁺ m/z: calcd 399.2; found 400.2; Rt=1.232 min.

### Step 6: Synthesis of (S)-5-(5-methyl-3,4,5,6-tetrahydropyridin-2-yl)-2-(1-methylazetidin-3-yl)benzo[d]thiazole

Prepared by general procedure scheme S2 step 4. Yield: 0.5 g (83.4%).

LCMS(ESI): [M]⁺ m/z: calcd 299.2; found 300.2; Rt=0.497 min.

### Step 7: Synthesis of 2-(1-methylazetidin-3-yl)-5-((2R,5S)-5-methylpiperidin-2-yl)benzo[d]thiazole)

Prepared by general procedure scheme S2 step 5. Yield: 0.35 g of crude.

LCMS(ESI): [M]⁺ m/z: calcd 301.2; found 302.2; Rt=0.556 min.

### Intermediate 7. 5-((2R,5S)-5-methylpiperidin-2-yl)-2-(1,2,2,6,6-pentamethylpiperidin-4-yl)benzo[d]thiazole

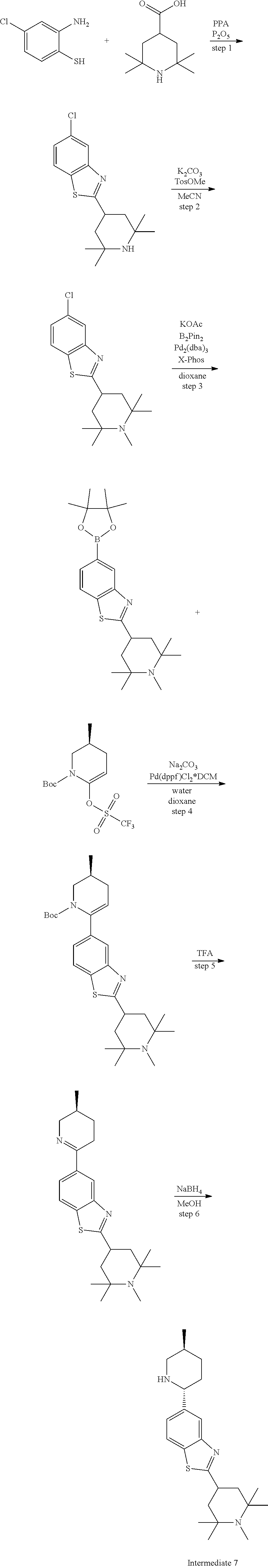

### Step 1: Synthesis of 5-chloro-2-(2,2,6,6-tetramethylpiperidin-4-yl)benzo[d]thiazole

Prepared by general procedure scheme S2 step 1A. Yield: 3.75 g (40.80%).

CC conditions: The crude product was purified by silica gel with MTBE/MeOH as an eluent mixture.

LCMS(ESI): [M]⁺ m/z: calcd 308.2; found 309.2; Rt=1.063 min.

### Step 2: Synthesis of 5-chloro-2-(1,2,2,6,6-pentamethylpiperidin-4-yl)benzo[d]thiazole

Methyl tosylate (2.35 g, 12.64 mmol, 1.91 mL) and potassium carbonate (3.18 g, 22.99 mmol, 1.39 mL) were added to the solution of 5-chloro-2-(2,2,6,6-tetramethyl-4-piperidyl)-1,3-benzothiazole (3.55 g, 11.49 mmol) in MeCN (50 mL). Resulting mixture was stirred at 80° C. for 48 hr. Then, solvent was removed under reduced pressure. Residue was suspended in MTBE (60 ml), filtered and concentrated in vacuum, leaving 5-chloro-2-(1,2,2,6,6-pentamethyl-4-piperidyl)-1,3-benzothiazole (3.89 g, crude). LCMS(ESI): [M]⁺ m/z: calcd 322.2; found 323.2; Rt=1.071 min.

### Step 3: Synthesis of 2-(1,2,2,6,6-pentamethylpiperidin-4-yl)-5-(4,4,5,5-tetramethyl-1,3,2-dioxaborolan-2-yl)benzo[d]thiazole

5-Chloro-2-(1,2,2,6,6-pentamethyl-4-piperidyl)-1,3-benzothiazole (3.89 g, 12.05 mmol), 4,4,5,5-tetramethyl-2-(4,4,5,5-tetramethyl-1,3,2-dioxaborolan-2-yl)-1,3,2-dioxaborolane (3.52 g, 13.85 mmol) and potassium acetate (2.36 g, 24.09 mmol, 1.51 mL) were mixed together in dioxane (60 mL). Reaction flask was evacuated and refilled with argon 3 times. Then, XPhos (1.15 g, 2.41 mmol) and tris(dibenzylideneacetone)dipalladium(0) (551.60 mg, 602.36 μmol) were added under stream of argon. Resulting mixture was stirred at 100° C. for 16 hr. Then, it was concentrated under reduced pressure and residue was purified by gradient column chromatography (SiO₂, MTBE/MeOH), affording 2-(1,2,2,6,6-pentamethyl-4-piperidyl)-5-(4,4,5,5-tetramethyl-1,3,2-dioxaborolan-2-yl)-1,3-benzothiazole (3 g, 7.24 mmol, 60.09% yield). LCMS(ESI): [M]⁺ m/z: calcd 414.2; found 415.2; Rt=1.144 min.

### Step 4: Synthesis of (S)-tert-butyl 3-methyl-6-(2-(1,2,2,6,6-pentamethylpiperidin-4-yl)benzo[d]thiazol-5-yl)-3,4-dihydropyridine-1(2H)-carboxylate

Prepared by general procedure scheme S2 step 3. Yield: 3.6 g of crude.

LCMS(ESI): [M]⁺ m/z: calcd 483.2; found 484.2; Rt=1.398 min.

### Step 5: Synthesis of (S)-5-(5-methyl-3,4,5,6-tetrahydropyridin-2-yl)-2-(1,2,2,6,6-pentamethylpiperidin-4-yl)benzo[d]thiazole

Prepared by general procedure scheme S2 step 4. Yield: 1.2 g of crude.

LCMS(ESI): [M]⁺ m/z: calcd 383.2; found 384.2; Rt=0.753 min.

### Step 6: Synthesis of 5-((2R,5S)-5-methylpiperidin-2-yl)-2-(1,2,2,6,6-pentamethylpiperidin-4-yl)benzo[d]thiazole

Prepared by general procedure scheme S2 step 5. Yield: 0.92 g of crude.

LCMS(ESI): [M]⁺ m/z: calcd 385.2; found 386.2; Rt=0.796 min.

### Intermediate 8. N,N-dimethyl-2-(5-((S)-5-methyl-3,4,5,6-tetrahydropyridin-2-yl)benzo[d]thiazol-2-yl) propan-1-amine

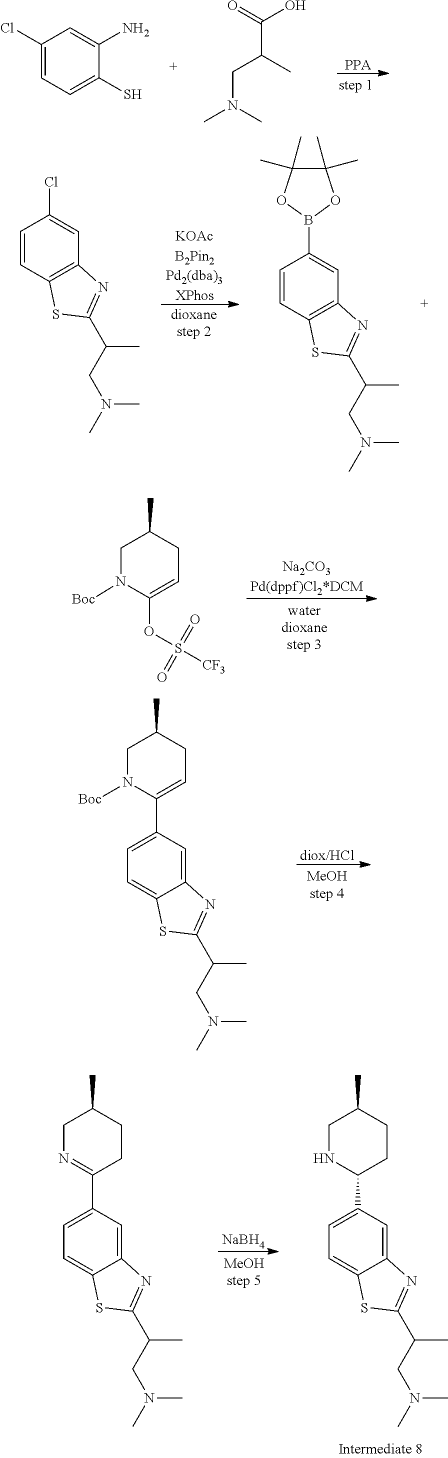

### Step 1: Synthesis of 2-(5-chlorobenzo[d]thiazol-2-yl)-N,N-dimethylpropan-1-amine

The stirred solution of 2-amino-4-chloro-benzenethiol (1.5 g, 9.40 mmol) and 2-amino-4-chloro-benzenethiol (1.5 g, 9.40 mmol) in PPA (10 mL) was allowed to stir at 140° C. for 16 hr. Upon completion, the reaction mixture was quenched with water (200 mL) and neutralized by NaOH to pH-8. The aqueous phase was extracted with EtOAc (2*20 mL). The combined organic phase was dried over Na₂SO₄ and concentrated under reduced pressure. The desired product 2-(5-chloro-1,3-benzothiazol-2-yl)-N,N-dimethyl-propan-1-amine (1.5 g, 5.89 mmol, 62.66% yield) was isolated. LCMS(ESI): [M]⁺ m/z: calcd 254.2; found 255.2; Rt=0.672 min.

### Step 2: Synthesis of N,N-dimethyl-2-(5-(4,4,5,5-tetramethyl-1,3,2-dioxaborolan-2-yl)benzo[d]thiazol-2-yl) propan-1-amine

To a stirred solution of 2-(5-chloro-1,3-benzothiazol-2-yl)-N,N-dimethyl-propan-1-amine (1.5 g, 5.89 mmol) and 4,4,5,5-tetramethyl-2-(4,4,5,5-tetramethyl-1,3,2-dioxaborolan-2-yl)-1,3,2-dioxaborolane (1.50 g, 5.89 mmol) in dioxane (30 mL) were added Pd₂(dba)₃ (1.08 g, 1.18 mmol) and XPhos (1.12 g, 2.35 mmol). The resulting suspension was degassed with argon at 50° C. for 0.5 hr. Potassium acetate (1.2 g, 12.23 mmol, 764.33 μL) was added. The reaction mixture was stirred at 100° C. for 16 hr. Upon completion, the reaction mixture was concentrated under reduced pressure, quenched with water (50 mL), the aqueous phase was extracted with CHCl₃ (2*50 mL). The organic phase was extracted with 10% HCl (2*50 ml). The aqueous phase was neutralized by NaHCO₃ to pH=8, extracted with CHCl₃ (2*50 mL). The organic phase was dried over Na₂SO₄ and evaporated in vacuum. The desired product N,N-dimethyl-2-[5-(4,4,5,5-tetramethyl-1,3,2-dioxaborolan-2-yl)-1,3-benzothiazol-2-yl]propan-1-amine (1.4 g, 4.04 mmol, 68.67% yield) was isolated. LCMS(ESI): [M]⁺ m/z: calcd 346.2; found 347.2; Rt=1.121 min.

### Step 3: Synthesis of (3S)-tert-butyl 6-(2-(1-(dimethylamino) propan-2-yl)benzo[d]thiazol-5-yl)-3-methyl-3,4-dihydropyridine-1(2H)-carboxylate

Prepared by general procedure scheme S2 step 3. Yield: 0.7 g of crude.

LCMS(ESI): [M]⁺ m/z: calcd 415.2; found 416.2; Rt=1.238 min.

### Step 4: Synthesis of N,N-dimethyl-2-(5-((S)-5-methyl-3,4,5,6-tetrahydropyridin-2-yl)benzo[d]thiazol-2-yl) propan-1-amine

The stirred solution of tert-butyl (3S)-6-[2-[2-(dimethylamino)-1-methyl-ethyl]-1,3-benzothiazol-5-yl]-3-methyl-3,4-dihydro-2H-pyridine-1-carboxylate (700.00 mg, 1.68 mmol) in MeOH (10 mL) and diox/HCl (10 mL) was allowed to stir at 25° C. for 16 hr. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The desired product N,N-dimethyl-2-[5-[(3S)-3-methyl-2,3,4,5-tetrahydropyridin-6-yl]-1,3-benzothiazol-2-yl]propan-1-amine (0.5 g, 1.58 mmol, 94.10% yield) was isolated. LCMS(ESI): [M]⁺ m/z: calcd 315.2; found 316.2; Rt=0.666 min.

### Step 5: Synthesis of N,N-dimethyl-2-(5-((2R,5S)-5-methylpiperidin-2-yl)benzo[d]thiazol-2-yl) propan-1-amine

Prepared by general procedure scheme S2 step 5. Yield: 0.5 g of crude.

LCMS(ESI): [M]⁺ m/z: calcd 317.2; found 318.2; Rt=0.471 min.

### Intermediate 9. 5-((2R,5S)-5-methylpiperidin-2-yl)-2-((1-methylpiperidin-4-yl)methyl)benzo[d]thiazole

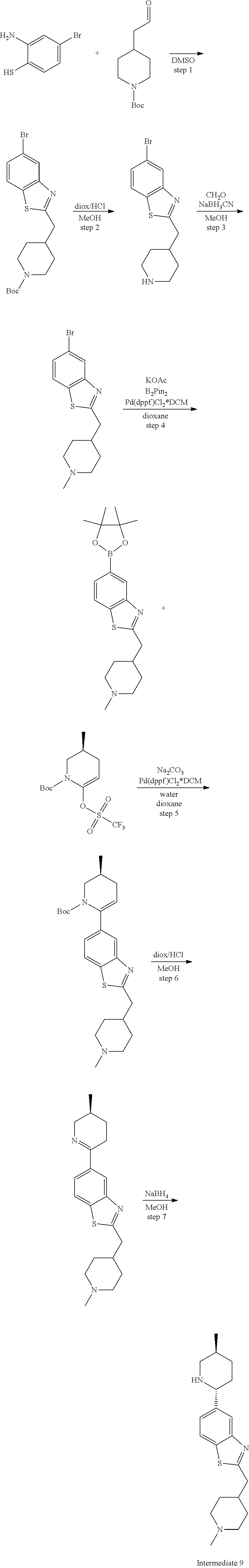

### Step 1: Synthesis of tert-butyl 4-((5-bromobenzo[d]thiazol-2-yl)methyl)piperidine-1-carboxylate

Prepared by general procedure scheme S2 step 1B. Yield: 2.1 g (38.68%).

LCMS(ESI): [M]⁺ m/z: calcd 411.2; found 412.2; Rt=1.128 min.

### Step 2: Synthesis of 5-bromo-2-(piperidin-4-ylmethyl)benzo[d]thiazole

tert-Butyl 4-[(5-bromo-1,3-benzothiazol-2-yl)methyl]piperidine-1-carboxylate (2.1 g, 5.11 mmol) was dissolved in mixture of MeOH (16.36 mL) and HCl in dioxane (102.10 mmol), then stirred for 1 hr. The reaction mixture was concentrated in vacuum, the residue was treated with aq. solution of NaHCO₃ and desired product was extracted with DCM (2*40 ml), dried over Na₂SO₄, evaporated in vacuum to give 5-bromo-2-(4-piperidylmethyl)-1,3-benzothiazole (1.6 g, 5.14 mmol, 100.70% yield). LCMS(ESI): [M]⁺ m/z: calcd 311.2; found 312.2; Rt=0.966 min.

### Step 3: Synthesis of 5-bromo-2-((1-methylpiperidin-4-yl)methyl)benzo[d]thiazole

Sodium cyan borohydride (1.62 g, 25.70 mmol) was added to a mixture of 5-bromo-2-(4-piperidylmethyl)-1,3-benzothiazole (1.6 g, 5.14 mmol) and formalin (2.09 g, 25.70 mmol, 1.93 mL, 37% purity) in MeOH (20 mL) and stirred overnight, then concentrated. The residue was treated with aq. solution of NaHCO₃ and desired product was extracted with DCM (2*30 ml), dried over Na₂SO₄ and concentrated in vacuum to give 5-bromo-2-[(1-methyl-4-piperidyl)methyl]-1,3-benzothiazole (1.5 g, 4.61 mmol, 89.71% yield). LCMS(ESI): [M]⁺ m/z: calcd 325.2; found 326.2; Rt=1.012 min.

### Step 4: Synthesis of 2-((1-methylpiperidin-4-yl)methyl)-5-(4,4,5,5-tetramethyl-1,3,2-dioxaborolan-2-yl)benzo[d]thiazole

Prepared by general procedure scheme S2 step 2. Yield: 1.8 g of crude.

LCMS(ESI): [M]⁺ m/z: calcd 372.2; found 373.2; Rt=1.139 min.

### Step 5: Synthesis of (S)-tert-butyl 3-methyl-6-(2-((1-methylpiperidin-4-yl)methyl)benzo[d]thiazol-5-yl)-3,4-dihydropyridine-1(2H)-carboxylate

Prepared by general procedure scheme S2 step 3. Yield: 2.1 g of crude.

LCMS(ESI): [M]⁺ m/z: calcd 441.2; found 442.2; Rt=1.231 min.

### Step 6: Synthesis of (S)-5-(5-methyl-3,4,5,6-tetrahydropyridin-2-yl)-2-((1-methylpiperidin-4-yl)methyl)benzo[d]thiazole

tert-Butyl (3S)-3-methyl-6-[2-[(1-methyl-4-piperidyl)methyl]-1,3-benzothiazol-5-yl]-3,4-dihydro-2H-pyridine-1-carboxylate (1.2 g, 2.72 mmol) was dissolved in MeOH (50 mL) and diox/HCl (54.34 mmol, 30 mL) was added thereto. Then it was stirred at rt for 2 hr. The reaction mixture was evaporated to afford 2-[(1-methyl-4-piperidyl)methyl]-5-[(3S)-3-methyl-2,3,4,5-tetrahydropyridin-6-yl]-1,3-benzothiazole (0.9 g, 2.64 mmol, 96.99% yield).

LCMS(ESI): [M]⁺ m/z: calcd 341.2; found 342.2; Rt=0.694 min.

### Step 7: Synthesis of 5-((2R,5S)-5-methylpiperidin-2-yl)-2-((1-methylpiperidin-4-yl)methyl)benzo[d]thiazole

Prepared by general procedure scheme S2 step 5. Yield: 0.98 g of crude.

LCMS(ESI): [M]⁺ m/z: calcd 343.2; found 344.2; Rt=0.637 min.

### Intermediate 10. N,N-dimethyl-3-(5-((2R,5S)-5-methylpiperidin-2-yl)benzo[d]thiazol-2-yl)cyclobutanamine

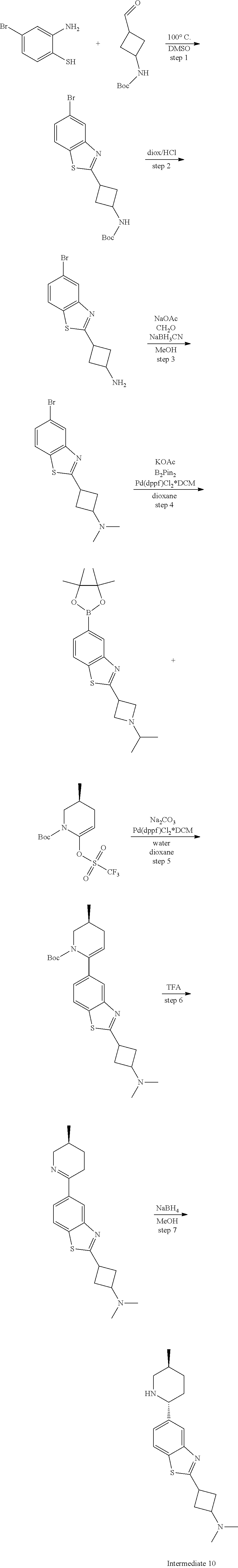

### Step 1: Synthesis of tert-butyl (3-(5-bromobenzo[d]thiazol-2-yl)cyclobutyl)carbamate

Prepared by general procedure scheme S2 step 1B. Yield: 5 g (53.24%).

LCMS(ESI): [M]⁺ m/z: calcd 383.2; found 384.2; Rt=1.402 min.

### Step 2: Synthesis of 3-(5-bromobenzo[d]thiazol-2-yl)cyclobutanamine

tert-Butyl N-[3-(5-bromo-1,3-benzothiazol-2-yl)cyclobutyl]carbamate (5 g, 13.04 mmol) was treated with hydrogen chloride solution 4.0M in dioxane (24.00 g, 658.26 mmol, 30 mL). The resulting mixture was stirred at 25° C. for 14 hr. Precipitate was filtered and additionally washed with MTBE. Then dried in vacuum to give 3-(5-bromo-1,3-benzothiazol-2-yl)cyclobutanamine (4 g, 12.51 mmol, 95.93% yield, HCl). LCMS(ESI): [M]⁺ m/z: calcd 284.2; found 285.2; Rt=0.931 min.

### Step 3: Synthesis of 3-(5-bromobenzo[d]thiazol-2-yl)-N,N-dimethylcyclobutanamine

To the stirred solution of 3-(5-bromo-1,3-benzothiazol-2-yl)cyclobutanamine (4 g, 12.51 mmol, HCl) in MeOH (71.00 mL) formaldehyde, 37% w/w aq. soln., stab. with 7-8% MeOH (2.54 g, 31.28 mmol, 2.34 mL, 37% purity) and sodium acetate, anhydrous (2.57 g, 31.28 mmol, 1.68 mL) were added. The resulting mixture was stirred for 2 hr at 25° C. Then Sodium cyan borohydride (1.57 g, 25.03 mmol) was added portion wise. The resulting mixture was stirred at 25° C. for 12 hr. MeOH was evaporated. The residue was diluted with water (100 ml) and extracted with DCM (3*50 ml). Combined organic layers were dried over Na₂SO₄. DCM was evaporated in vacuum to give 3-(5-bromo-1,3-benzothiazol-2-yl)-N,N-dimethylcyclobutanamine (3.9 g, crude). LCMS(ESI): [M]⁺ m/z: calcd 311.2; found 312.2; Rt=0.829 min.

### Step 4: Synthesis of N,N-dimethyl-3-(5-(4,4,5,5-tetramethyl-1,3,2-dioxaborolan-2-yl)benzo[d]thiazol-2-yl)cyclobutanamine

Prepared by general procedure scheme S2 step 2. Yield: 4 g of crude.

LCMS(ESI): [M]⁺ m/z: calcd 358.2; found 359.2; Rt=1.029 min.

### Step 5: Synthesis of (S)-tert-butyl 6-(2-(3-(dimethylamino)cyclobutyl)benzo[d]thiazol-5-yl)-3-methyl-3,4-dihydropyridine-1(2H)-carboxylate

Prepared by general procedure scheme S2 step 3. Yield: 7 g of crude.

LCMS(ESI): [M]⁺ m/z: calcd 427.2; found 428.2; Rt=1.057 min.

### Step 6: Synthesis of (S)—N,N-dimethyl-3-(5-(5-methyl-3,4,5,6-tetrahydropyridin-2-yl)benzo[d]thiazol-2-yl)cyclobutanamine

Prepared by general procedure scheme S2 step 4. Yield: 3 g of crude.

LCMS(ESI): [M]⁺ m/z: calcd 327.2; found 328.2; Rt=0.635 min.

### Step 7: Synthesis of N,N-dimethyl-3-(5-((2R,5S)-5-methylpiperidin-2-yl)benzo[d]thiazol-2-yl)cyclobutanamine

Prepared by general procedure scheme S2 step 5. Yield: 80 mg (2.65%).

HPLC conditions: Column: Chromatorex C18 100*19 mm, 5 microM; 0-1-6 min 45-45-85% water-MeOH, flow: 30 ml/min; (loading pump 4 ml/min MeOH).

LCMS(ESI): [M]⁺ m/z: calcd 329.2; found 330.2; Rt=1.324 min.

### Intermediate 11. 5-((2R,5S)-5-methylpiperidin-2-yl)-2-(1,3,3-trimethylpiperidin-4-yl)benzo[d]thiazole

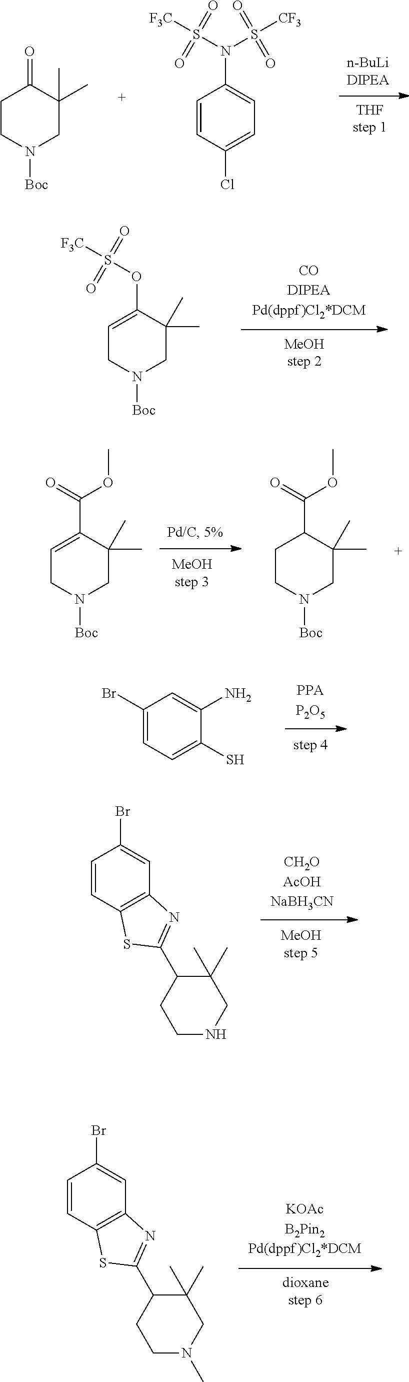

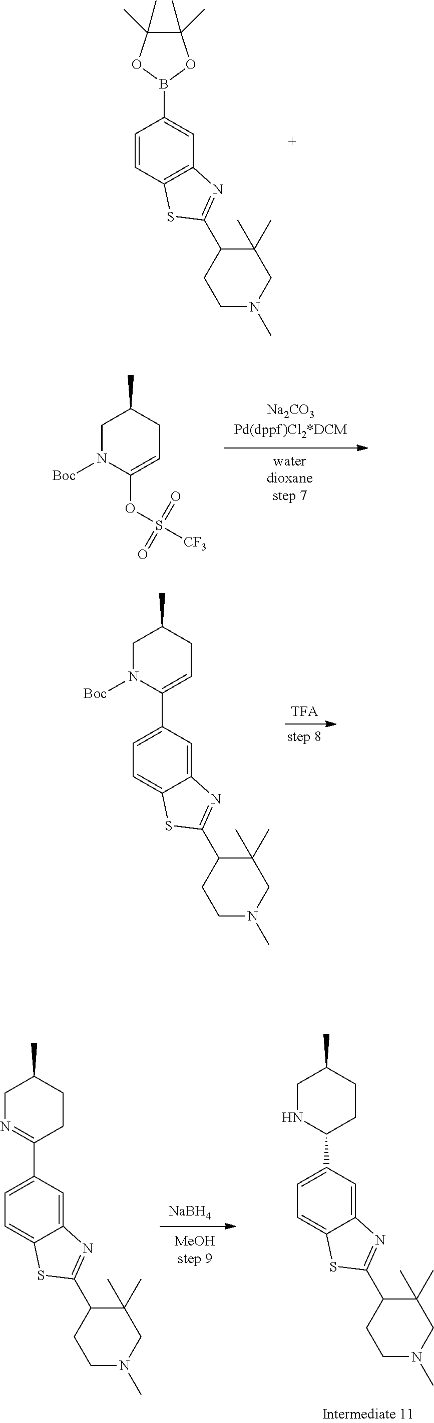

### Step 1: Synthesis of tert-butyl 5,5-dimethyl-4-(((trifluoromethyl) sulfonyl)oxy)-5,6-dihydropyridine-1(2H)-carboxylate

LDA was generated in situ by reacting a solution of DIPEA (14.47 g, 142.98 mmol, 20.15 mL) in 200 mL tetrahydrofuran with n-Butyllithium, 2.2M in hexane, packaged under Argon in resalable ChemSeal bottles (8.45 g, 131.98 mmol, 60 mL). After cooling this solution to −78° C., a solution of tert-butyl 3,3-dimethyl-4-oxo-piperidine-1-carboxylate (25 g, 109.99 mmol) in 100 ml THF was added dropwise, maintaining the reaction temperature below −70° C. After the addition was complete, the reaction mixture was stirred at −78° C. for 1 hr, and then a solution of N-(5-chloro-2-pyridyl)-1,1,1-trifluoro-N-(trifluoromethylsulfonyl) methanesulfonamide (43.19 g, 109.99 mmol) in 100 mL tetrahydrofuran was added over 5 min. The solution was allowed to warm to 0° C. and was stirred for 90 min and poured into 150 ml NH₄Cl water solution, stirred for 10 min and extracted with EtOAc (3*30 ml), combined organic layers were dried over Na₂SO₄, filtered and concentrated in vacuum. The crude product was purified by silica gel column chromatography using EtOAc:Hex (1:9, v:v) as eluent (Rt=0.5) to afford tert-butyl 3,3-dimethyl-4-(trifluoromethylsulfonyloxy)-2,6-dihydropyridine-1-carboxylate (18 g, 50.09 mmol, 45.54% yield). LCMS(ESI): [M]⁺ m/z: calcd 359.2; found 360.2; Rt=1.650 min.

### Step 2: Synthesis of 1-tert-butyl 4-methyl 5,5-dimethyl-5,6-dihydropyridine-1,4 (2H)-dicarboxylate

tert-Butyl 3,3-dimethyl-4-(trifluoromethylsulfonyloxy)-2,6-dihydropyridine-1-carboxylate (18 g, 50.09 mmol), DIPEA (7.77 g, 60.11 mmol, 10.47 mL) and Pd(dppf)Cl₂*DCM (2.05 g, 2.50 mmol) were dissolved in dry MeOH (350 mL). The reaction mixture was heated at 80° C. in high pressure vessel at 40 atm CO (48.15 g, 1.50 mol, 60.87 mL) pressure for 17 hr. Then the solvent was evaporated to dryness and the mixture was poured into 200 ml of water. The mixture was extracted with DCM (3×100 ml), organics were dried over Na₂SO₄, filtered through the thin SiO₂ pad and evaporated to dryness affording O1-tert-butyl O4-methyl 3,3-dimethyl-2,6-dihydropyridine-1,4-dicarboxylate (15 g, crude).

LCMS(ESI): [M]⁺ m/z: calcd 269.2; found 270.2; Rt=1.486 min.

### Step 3: Synthesis of 1-tert-butyl 4-methyl 3,3-dimethylpiperidine-1,4-dicarboxylate

O1-tert-butyl O4-methyl 3,3-dimethyl-2,6-dihydropyridine-1,4-dicarboxylate (15 g, 55.69 mmol) was dissolved in MeOH (200 mL) and palladium, 5% on activated carbon paste, 5R437 (4 g, 37.59 mmol) was added. The mixture was stirred under H₂ at 50° C. and 40 psi for 70 hr, filtered through celite, and evaporated to dryness to obtain O1-tert-butyl O4-methyl 3,3-dimethylpiperidine-1,4-dicarboxylate (10 g, crude). LCMS(ESI): [M]⁺ m/z: calcd 271.2; found 272.2; Rt=1.347 min.

### Step 4: Synthesis of 5-bromo-2-(3,3-dimethylpiperidin-4-yl)benzo[d]thiazole)

Prepared by general procedure scheme S2 step 1A. Yield: 1.8 g of crude.

LCMS(ESI): [M]⁺ m/z: calcd 325.2; found 326.2; Rt=1.044 min.

### Step 5: Synthesis of 5-bromo-2-(1,3,3-trimethylpiperidin-4-yl)benzo[d]thiazole

Formaldehyde, 37% w/w aq. soln., stab. with 7-8% MeOH (597.72 mg, 19.91 mmol, 551.91 μL) and acetic acid (683.11 mg, 11.38 mmol, 651.20 μL) were added to a stirred solution of 5-bromo-2-(3,3-dimethyl-4-piperidyl)-1,3-benzothiazole (1.85 g, 5.69 mmol) in MeOH (50 mL) at 25° C. The resulting mixture was stirred at 25° C. 0.5 hr, then sodium cyan borohydride (714.85 mg, 11.38 mmol) was added in one portion at 25° C. (foaming!). The reaction mixture was stirred at 25° C. for 18 hr, and then concentrated in vacuum. The residue was diluted with 10% aqueous sodium hydroxide solution (20 ml) and extracted with DCM (2*20 ml). The combined organic extracts were dried over sodium sulphate and concentrated in vacuum to afford crude 5-bromo-2-(1,3,3-trimethyl-4-piperidyl)-1,3-benzothiazole (1.3 g, crude). LCMS(ESI): [M]⁺ m/z: calcd 339.2; found 340.2; Rt=1.040 min.

### Step 6: Synthesis of 5-(4,4,5,5-tetramethyl-1,3,2-dioxaborolan-2-yl)-2-(1,3,3-trimethylpiperidin-4-yl)benzo[d]thiazole

Prepared by general procedure scheme S2 step 2. Yield: 1.6 g of crude.

LCMS(ESI): [M]⁺ m/z: calcd 386.2; found 387.2; Rt=0.944 min.

### Step 7: Synthesis of (3S)-tert-butyl 3-methyl-6-(2-(1,3,3-trimethylpiperidin-4-yl)benzo[d]thiazol-5-yl)-3,4-dihydropyridine-1(2H)-carboxylate

Prepared by general procedure scheme S2 step 3. Yield: 1.2 g of crude.

LCMS(ESI): [M]⁺ m/z: calcd 455.2; found 456.2; Rt=1.316 min.

### Step 8: Synthesis of 5-((S)-5-methyl-3,4,5,6-tetrahydropyridin-2-yl)-2-(1,3,3-trimethylpiperidin-4-yl)benzo[d]thiazole

Prepared by general procedure scheme S2 step 4. Yield: 0.4 g of crude.

LCMS(ESI): [M]⁺ m/z: calcd 355.2; found 356.2; Rt=0.726 min.

### Step 9: Synthesis of 5-((2R,5S)-5-methylpiperidin-2-yl)-2-(1,3,3-trimethylpiperidin-4-yl)benzo[d]thiazole

Prepared by general procedure scheme S2 step 5. Yield: 0.5 g of crude.

LCMS(ESI): [M]⁺ m/z: calcd 357.2; found 358.2; Rt=0.634 min.

### Intermediate 12. 2-(2-methyl-2-azabicyclo[2.2.2]octan-4-yl)-5-((2R,5S)-5-methylpiperidin-2-yl)benzo[d]thiazole

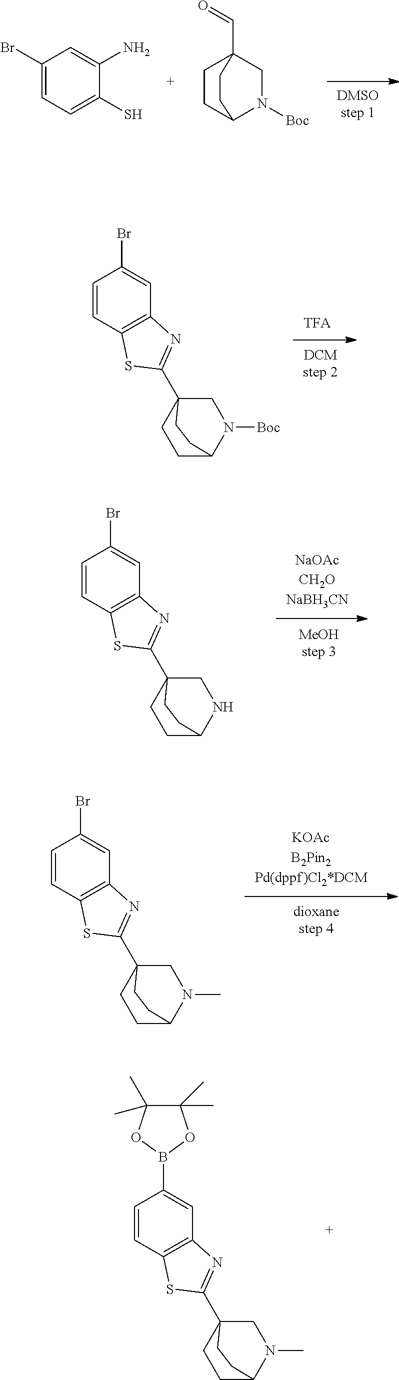

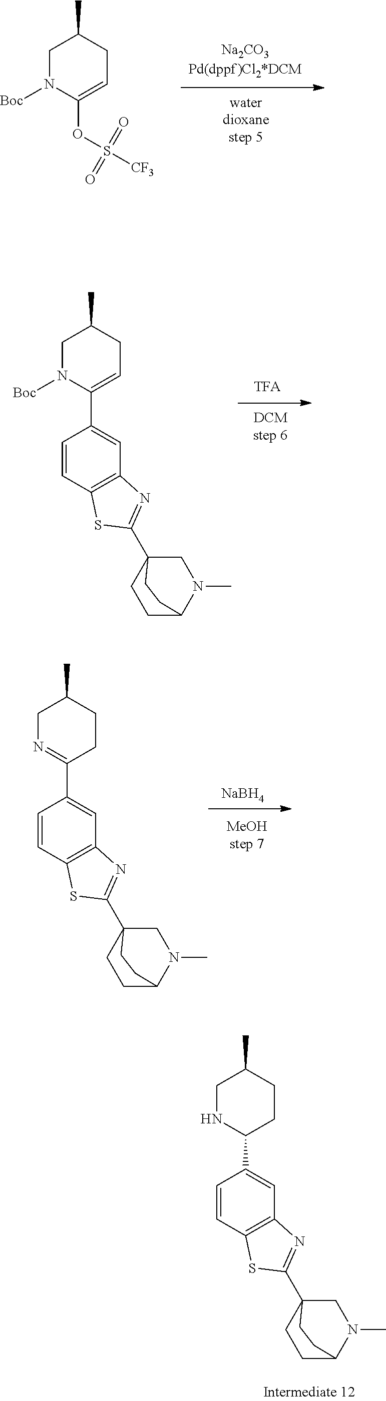 [SMILES: C[C@H]1CC=C(OS(=O)(=O)C(F)(F)F)N(C(=O)OC(C)(C)C)C1 ; C[C@H]1CC=C(c2ccc3sc(C45CCC(CC4)N(C)C5)nc3c2)N(C(=O)OC(C)(C)C)C1 ; C[C@H]1CCC(c2ccc3sc(C45CCC(CC4)N(C)C5)nc3c2)=NC1 ; "NaBH4" ; C[C@H]1CC[C@H](c2ccc3sc(C45CCC(CC4)N(C)C5)nc3c2)NC1]

### Step 1: Synthesis of tert-butyl 4-(5-bromobenzo[d]thiazol-2-yl)-2-azabicyclo[2.2.2]octane-2-carboxylate

Prepared by general procedure scheme S2 step 1B. Yield: 2.82 g (79.7%).

LCMS(ESI): [M]⁺ m/z: calcd 423.2; found 424.2; Rt=1.572 min.

### Step 2: Synthesis of 2-(2-azabicyclo[2.2.2]octan-4-yl)-5-bromobenzo[d]thiazole

TFA (7.56 g, 66.29 mmol, 5.11 mL) was added to a solution of tert-butyl 4-(5-bromo-3a,7a-dihydro-1,3-benzothiazol-2-yl)-2-azabicyclo[2.2.2]octane-2-carboxylate (2.82 g, 6.63 mmol) in DCM (20 mL). Resulting mixture was stirred at 20° C. for 4 hr. Then, volatiles were removed under reduced pressure, leaving 2-(2-azabicyclo[2.2. 2]octan-4-yl)-5-bromo-1,3-benzothiazole (3 g, crude, TFA).

LCMS(ESI): [M]⁺ m/z: calcd 323.2; found 324.2; Rt=1.025 min.

### Step 3: Synthesis of 5-bromo-2-(2-methyl-2-azabicyclo[2.2.2]octan-4-yl)benzo[d]thiazole

Formaldehyde, 37% w/w aq. soln., stab. with 7-8% MeOH (1.12 g, 13.75 mmol, 1.03 mL, 37% purity) and sodium acetate (1.69 g, 20.63 mmol, 1.11 mL) were added to the solution of 2-(2-azabicyclo[2.2.2]octan-4-yl)-5-bromo-1,3-benzothiazole (3 g, 6.88 mmol, TFA) in MeOH (50 mL). Resulting mixture was stirred at 20° C. for 1 hr before sodium cyan borohydride (864.27 mg, 13.75 mmol) was added thereto. After that, stirring was continued for 18 hr. Then, solvent was removed under reduced pressure and residue was partitioned between 15% aq. K₂CO₃ solution (30 ml) and DCM (50 ml). Organic layer was separated, dried over solid K₂CO₃ and concentrated under reduced pressure, affording 5-bromo-2-(2-methyl-2-azabicyclo[2.2.2]octan-4-yl)-1,3-benzothiazole (2.37 g, crude). LCMS(ESI): [M]⁺ m/z: calcd 337.2; found 338.2; Rt=1.026 min.

### Step 4: Synthesis of 2-(2-methyl-2-azabicyclo[2.2.2]octan-4-yl)-5-(4,4,5,5-tetramethyl-1,3,2-dioxaborolan-2-yl)benzo[d]thiazole

Prepared by general procedure scheme S2 step 2. Yield: 2.95 g of crude.

LCMS(ESI): [M]⁺ m/z: calcd 384.2; found 385.2; Rt=1.183 min.

### Step 5: Synthesis of (S)-tert-butyl 3-methyl-6-(2-(2-methyl-2-azabicyclo[2.2.2]octan-4-yl)benzo[d]thiazol-5-yl)-3,4-dihydropyridine-1(2H)-carboxylate

Prepared by general procedure scheme S2 step 3. Yield: 4.14 g of crude.

LCMS(ESI): [M]⁺ m/z: calcd 453.2; found 454.2; Rt=1.189 min.

### Step 6: Synthesis of (S)-5-(5-methyl-3,4,5,6-tetrahydropyridin-2-yl)-2-(2-methyl-2-azabicyclo[2.2.2]octan-4-yl)benzo[d]thiazole

Prepared by general procedure scheme S2 step 4. Yield: 1.98 g of crude.

LCMS(ESI): [M]⁺ m/z: calcd 353.2; found 354.2; Rt=0.723 min.

### Step 7: Synthesis of 2-(2-methyl-2-azabicyclo[2.2.2]octan-4-yl)-5-((2R,5S)-5-methylpiperidin-2-yl)benzo[d]thiazole

Prepared by general procedure scheme S2 step 5. Yield: 1.71 g (85.87%).

LCMS(ESI): [M]⁺ m/z: calcd 355.2; found 356.2; Rt=0.781 min.

### Intermediate 13. 2-(2-methyl-2-azaspiro[3.3]heptan-6-yl)-5-((2R,5S)-5-methylpiperidin-2-yl)benzo[d]thiazole

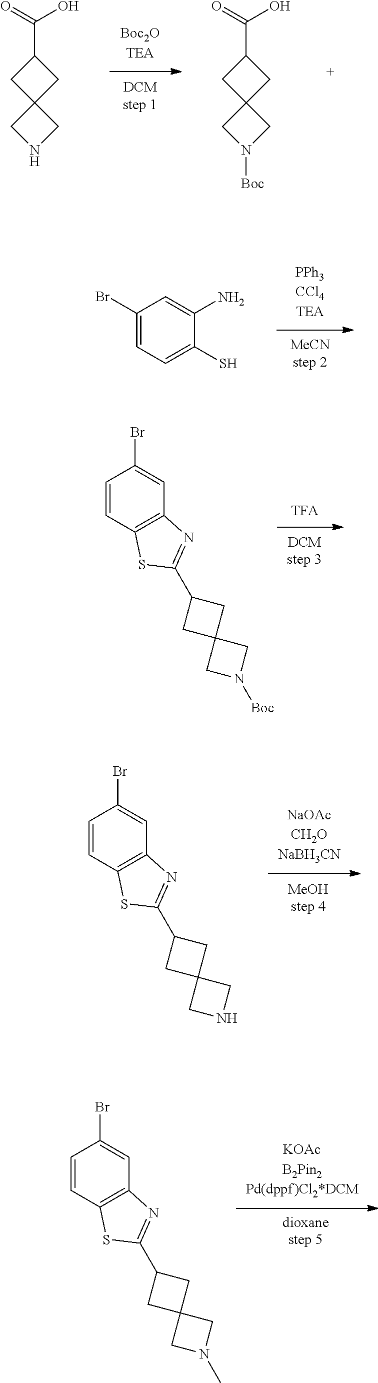 [SMILES: O=C(O)C1CC2(CNC2)C1 ; CC(C)(C)OC(=O)N1CC2(CC(C(=O)O)C2)C1 ; Nc1cc(Br)ccc1S ; CC(C)(C)OC(=O)N1CC2(CC(c3nc4cc(Br)ccc4s3)C2)C1 ; Brc1ccc2sc(C3CC4(CNC4)C3)nc2c1 ; CN1CC2(CC(c3nc4cc(Br)ccc4s3)C2)C1]

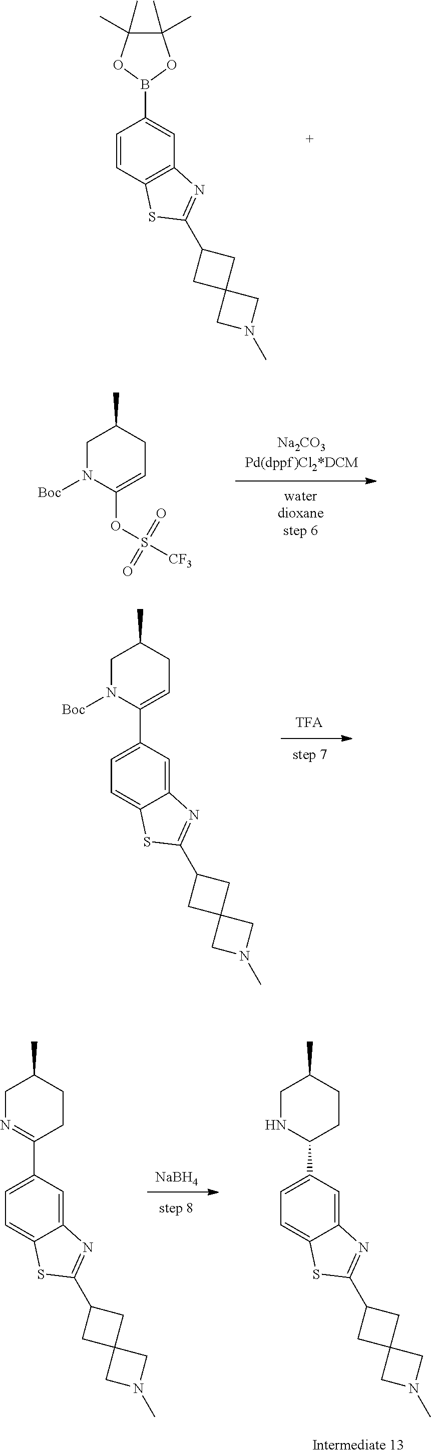

### Step 1: Synthesis of 2-(tert-butoxycarbonyl)-2-azaspiro[3.3]heptane-6-carboxylic acid

Di-tert-butyl dicarbonate (7.88 g, 36.12 mmol, 8.29 mL) was added dropwise to the suspension of 2-azaspiro[3.3]heptane-6-carboxylic acid (6.11 g, 34.40 mmol, HCl) in DCM (102.40 mL) and TEA (8.70 g, 85.99 mmol, 11.99 mL). Resulting reaction mixture was stirred at 20° C. for 16 hr. Then, 10% aq. NaHSO₄ solution (80 ml) was added and stirring was continued for 5 min. After that, organic layer was separated, dried over Na₂SO₄ and concentrated under reduced pressure, leaving 2-tert-butoxycarbonyl-2-azaspiro[3.3]heptane-6-carboxylic acid (8.8 g, crude). ¹H NMR (400 MHz, CDCl₃) δ (ppm) 1.41 (s, 9H), 1.51 (m, 1H), 2.43 (m, 4H), 3.00 (m, 1H), 3.90 (m, 4H).

### Step 2: Synthesis of tert-butyl 6-(5-bromobenzo[d]thiazol-2-yl)-2-azaspiro[3.3]heptane-2-carboxylate

Triphenylphosphine (516.35 mg, 1.97 mmol) was added in one portion to the solution of 2-tert-butoxycarbonyl-2-azaspiro[3.3]heptane-6-carboxylic acid (190 mg, 787.46 μmol), 2-amino-4-bromo-benzenethiol (160.71 mg, 787.46 μmol), carbon tetrachloride (795.00 mg, 5.17 mmol, 0.5 mL) and TEA (398.41 mg, 3.94 mmol, 548.78 μL). Resulting reaction mixture was briefly warmed up to approximately 50-60° C. due to exothermic reaction. After that, it was stirred at 20° C. for 18 hr. Then, volatiles were removed under reduced pressure and residue was triturated with MTBE (100 ml). Resulting light precipitate was filtered off. Filtrate was concentrated under reduced pressure and residue was purified by gradient column chromatography (SiO₂, Hexane/EtOAc), affording tert-butyl 6-(5-bromo-1,3-benzothiazol-2-yl)-2-azaspiro[3.3]heptane-2-carboxylate (4.6 g, 11.24 mmol, 31.53% yield). LCMS(ESI): [M]⁺ m/z: calcd 409.2; found 410.2; Rt=1.581 min.

### Step 3: Synthesis of 5-bromo-2-(2-azaspiro[3.3]heptan-6-yl)benzo[d]thiazole

Trifluoroacetic acid (12.81 g, 112.38 mmol, 8.66 mL) was added to the solution of tert-butyl 6-(5-bromo-1,3-benzothiazol-2-yl)-2-azaspiro[3.3]heptane-2-carboxylate (4.6 g, 11.24 mmol) in DCM (60 mL). Resulting mixture was stirred at 20° C. for 5 hr. Then, it was concentrated under reduced pressure, leaving 2-(2-azaspiro[3.3]heptan-6-yl)-5-bromo-1,3-benzothiazole (5 g, crude, TFA). LCMS(ESI): [M]⁺ m/z: calcd 309.2; found 310.2; Rt=0.830 min.

### Step 4: Synthesis of 5-bromo-2-(2-methyl-2-azaspiro[3.3]heptan-6-yl)benzo[d]thiazole

Formaldehyde, 37% w/w aq. soln., stab. with 7-8% MeOH (711.21 mg, 23.68 mmol, 656.70 μL) and sodium acetate (1.94 g, 23.68 mmol, 1.27 mL) were added to the solution of 2-(2-azaspiro[3.3]heptan-6-yl)-5-bromo-1,3-benzothiazole (5 g, 11.84 mmol, TFA) in MeOH (60 mL). Resulting mixture was stirred at 20° C. for 1 hr before sodium cyan borohydride (1.49 g, 23.68 mmol) was added thereto. After that, stirring was continued for 16 hr. Then, solvent was removed under reduced pressure and residue was partitioned between 15% aq. K₂CO₃ solution (30 ml) and DCM (50 ml). Organic layer was separated, dried over solid K₂CO₃ and concentrated under reduced pressure, leaving 5-bromo-2-(2-methyl-2-azaspiro[3.3]heptan-6-yl)-1,3-benzothiazole (3.8 g, 11.76 mmol, 99.27% yield). LCMS(ESI): [M]⁺ m/z: calcd 323.2; found 324.2; Rt=1.079 min.

### Step 5: Synthesis of 2-(2-methyl-2-azaspiro[3.3]heptan-6-yl)-5-(4,4,5,5-tetramethyl-1,3,2-dioxaborolan-2-yl)benzo[d]thiazole

Prepared by general procedure scheme S2 step 2. Yield: 1.5 g (34.46%).

CC conditions: The crude product was purified by silica gel with MTBE/MeOH as an eluent mixture.

LCMS(ESI): [M]⁺ m/z: calcd 370.2; found 371.2; Rt=2.997 min.

### Step 6: Synthesis of (S)-tert-butyl 3-methyl-6-(2-(2-methyl-2-azaspiro[3.3]heptan-6-yl)benzo[d]thiazol-5-yl)-3,4-dihydropyridine-1(2H)-carboxylate

Prepared by general procedure scheme S2 step 3. Yield: 1.2 g of crude.

LCMS(ESI): [M]⁺ m/z: calcd 439.2; found 440.2; Rt=1.166 min.

### Step 7: Synthesis of (S)-5-(5-methyl-3,4,5,6-tetrahydropyridin-2-yl)-2-(2-methyl-2-azaspiro[3.3]heptan-6-yl)benzo[d]thiazole

Prepared by general procedure scheme S2 step 4. Yield: 700 mg of crude.

LCMS(ESI): [M]⁺ m/z: calcd 339.2; found 340.2; Rt=0.774 min.

### Step 8: Synthesis of 2-(2-methyl-2-azaspiro[3.3]heptan-6-yl)-5-((2R,5S)-5-methylpiperidin-2-yl)benzo[d]thiazole

Prepared by general procedure scheme S2 step 5. Yield: 600 mg (72.91%).

LCMS(ESI): [M]⁺ m/z: calcd 341.2; found 342.2; Rt=0.770 min.

### Intermediate 14. Synthesis of 2-(1-azabicyclo[2.2. 1]heptan-4-yl)-5-((2R,5S)-5-methylpiperidin-2-yl)benzo[d]thiazole

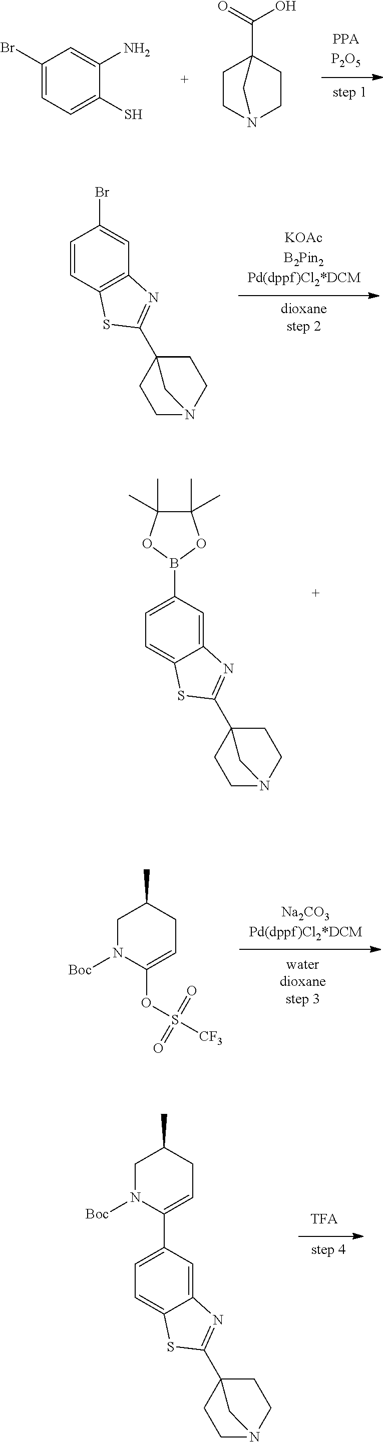 [SMILES: Nc1cc(Br)ccc1S ; O=C(O)C12CCN(CC1)C2 ; Brc1ccc2sc(C34CCN(CC3)C4)nc2c1 ; CC1(C)OB(c2ccc3sc(C45CCN(CC4)C5)nc3c2)OC1(C)C ; C[C@H]1CC=C(OS(=O)(=O)C(F)(F)F)N(C(=O)OC(C)(C)C)C1 ; C[C@H]1CC=C(c2ccc3sc(C45CCN(CC4)C5)nc3c2)N(C(=O)OC(C)(C)C)C1]

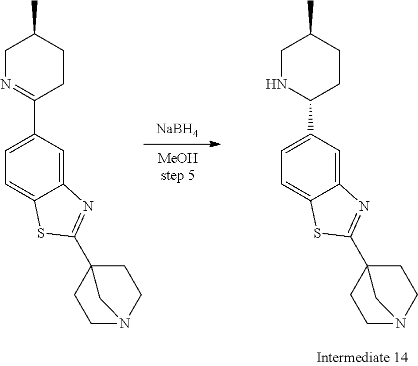 [SMILES: C[C@H]1CCC(c2ccc3sc(C45CCN(CC4)C5)nc3c2)=NC1 ; "NaBH4" ; C[C@H]1CC[C@H](c2ccc3sc(C45CCN(CC4)C5)nc3c2)NC1]

### Step 1: Synthesis of 2-(1-azabicyclo[2.2.1]heptan-4-yl)-5-bromobenzo[d]thiazole

Prepared by general procedure scheme S2 step 1A. Yield: 3.5 g (92.4%).

LCMS(ESI): [M]⁺ m/z: calcd 309.2; found 310.2; Rt=0.961 min.

### Step 2: Synthesis of 2-(1-azabicyclo[2.2.1]heptan-4-yl)-5-(4,4,5,5-tetramethyl-1,3,2-dioxaborolan-2-yl)benzo[d]thiazole

Prepared by general procedure scheme S2 step 2. Yield: 4 g of crude.

LCMS(ESI): [M]⁺ m/z: calcd 356.2; found 357.2; Rt=1.025 min.

### Step 3: Synthesis of (S)-tert-butyl 6-(2-(1-azabicyclo[2.2.1]heptan-4-yl)benzo[d]thiazol-5-yl)-3-methyl-3,4-dihydropyridine-1(2H)-carboxylate

Prepared by general procedure scheme S2 step 3. Yield: 8 g of crude.

LCMS(ESI): [M]⁺ m/z: calcd 425.2; found 426.2; Rt=1.254 min.

### Step 4: Synthesis of (S)-2-(1-azabicyclo[2.2.1]heptan-4-yl)-5-(5-methyl-3,4,5,6-tetrahydropyridin-2-yl)benzo[d]thiazole

Prepared by general procedure scheme S2 step 4. Yield: 6 g of crude.

LCMS(ESI): [M]⁺ m/z: calcd 325.2; found 326.2; Rt=0.683 min.

### Step 5: Synthesis of 2-(1-azabicyclo[2.2.1]heptan-4-yl)-5-((2R,5S)-5-methylpiperidin-2-yl)benzo[d]thiazole

Prepared by general procedure scheme S2 step 5. Yield: 2 g of crude.

LCMS(ESI): [M]⁺ m/z: calcd 327.2; found 328.2; Rt=0.709 min.

### Intermediate 15. 4-methyl-2-(5-((2R,5S)-5-methylpiperidin-2-yl)benzo[d]thiazol-2-yl) morpholine

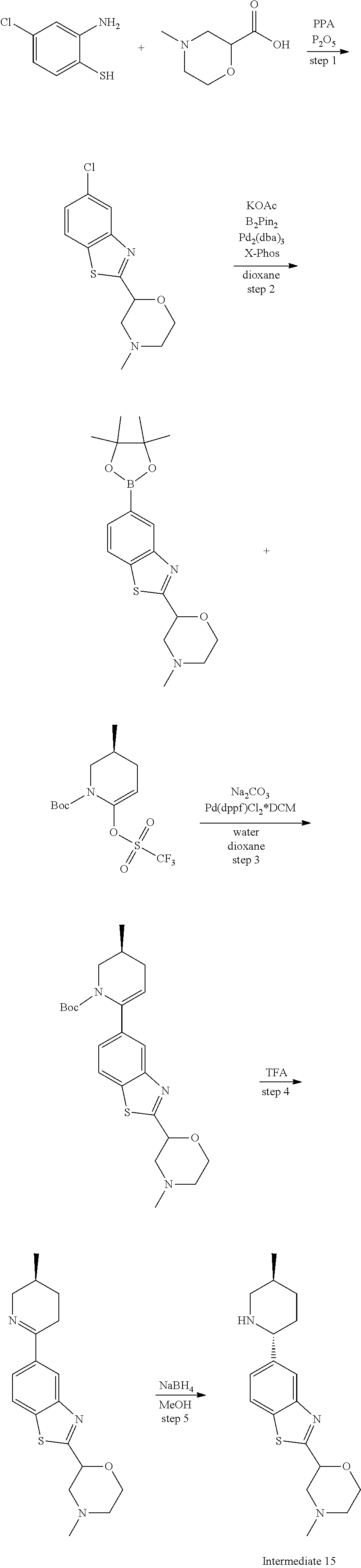

### Step 1: Synthesis of 2-(5-chlorobenzo[d]thiazol-2-yl)-4-methylmorpholine

Prepared by general procedure scheme S2 step 1A. Yield: 11.5 g of crude.

LCMS(ESI): [M]⁺ m/z: calcd 268.2; found 269.2; Rt=0.966 min.

### Step 2: Synthesis of 4-methyl-2-(5-(4,4,5,5-tetramethyl-1,3,2-dioxaborolan-2-yl)benzo[d]thiazol-2-yl) morpholine

2-(5-Chloro-1,3-benzothiazol-2-yl)-4-methyl-morpholine (11.5 g, 42.79 mmol), 4,4,5,5-tetramethyl-2-(4,4,5,5-tetramethyl-1,3,2-dioxaborolan-2-yl)-1,3,2-dioxaborolane (11.95 g, 47.07 mmol) and potassium acetate (8.40 g, 85.58 mmol, 5.35 mL) were mixed in dioxane (150 mL). The resulting mixture was evacuated and then backfilled with argon, this operation was repeated three times, then tris(dibenzylideneacetone)dipalladium(0) (1.57 g, 1.71 mmol) and XPhos (3.26 g, 6.85 mmol) was added under argon. The reaction mixture was stirred under argon at 95° C. for 18 hr, then cooled, filtered and concentrated under reduce pressure. The residue was purified by gradient chromatography (MTBE-MeOH) to give 4-methyl-2-[5-(4,4,5,5-tetramethyl-1,3,2-dioxaborolan-2-yl)-1,3-benzothiazol-2-yl]morpholine (11 g, 30.53 mmol, 71.36% yield). LCMS(ESI): [M]⁺ m/z: calcd 360.2; found 361.2; Rt=1.012 min.

### Step 3: Synthesis of (3S)-tert-butyl 3-methyl-6-(2-(4-methylmorpholin-2-yl)benzo[d]thiazol-5-yl)-3,4-dihydropyridine-1(2H)-carboxylate

Prepared by general procedure scheme S2 step 3. Yield: 3 g of crude.

LCMS(ESI): [M]⁺ m/z: calcd 429.2; found 430.2; Rt=1.232 min.

### Step 4: Synthesis of 4-methyl-2-(5-((S)-5-methyl-3,4,5,6-tetrahydropyridin-2-yl)benzo[d]thiazol-2-yl) morpholine

Prepared by general procedure scheme S2 step 4. Yield: 1.3 g of crude.

LCMS(ESI): [M]⁺ m/z: calcd 329.2; found 330.2; Rt=0.673 min.

### Step 5: Synthesis of 4-methyl-2-(5-((2R,5S)-5-methylpiperidin-2-yl)benzo[d]thiazol-2-yl) morpholine

Prepared by general procedure scheme S2 step 5. Yield: 0.9 g of crude.

LCMS(ESI): [M]⁺ m/z: calcd 331.2; found 332.2; Rt=0.695 min.

### Intermediate 16. 2-((2R,5S)-5-methyl-2-(2-(1-(oxetan-3-yl)piperidin-4-yl)benzo[d]thiazol-5-yl)piperidin-1-yl)-2-oxoacetamide

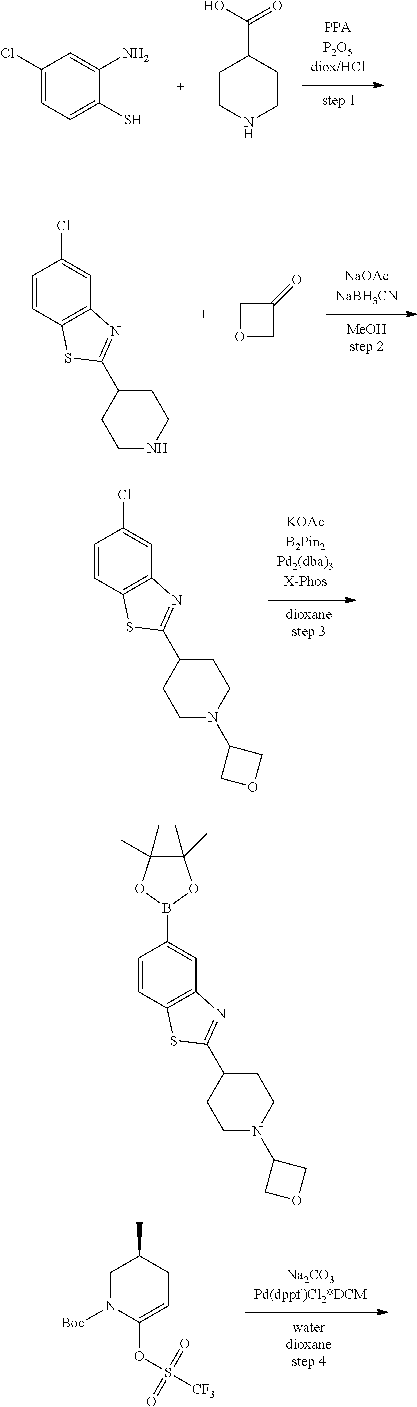

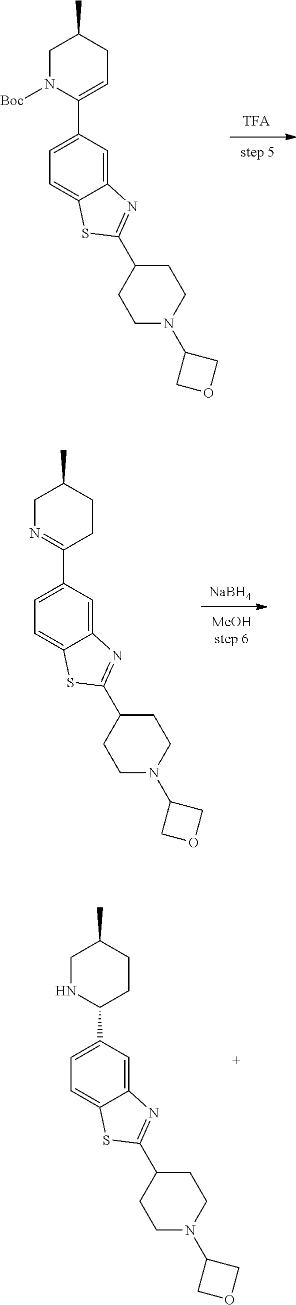 [SMILES: C[C@H]1CC=C(c2ccc3sc(C4CCN(C5COC5)CC4)nc3c2)N(C(=O)OC(C)(C)C)C1 ; C[C@H]1CCC(c2ccc3sc(C4CCN(C5COC5)CC4)nc3c2)=NC1 ; "NaBH4" ; C[C@H]1CC[C@H](c2ccc3sc(C4CCN(C5COC5)CC4)nc3c2)NC1]

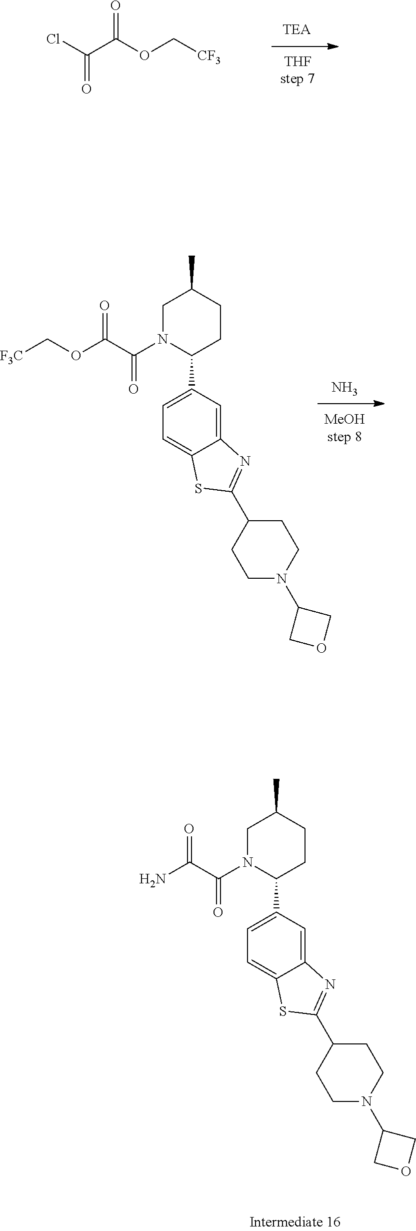

### Step 1: Synthesis of 5-chloro-2-(piperidin-4-yl)benzo[d]thiazole

Phosphorus (V) pentoxide (50 g, 352.25 mmol) was added portion wise with stirring to a phosphoric acid (60 g, 612.27 mmol, 35.29 mL). The resulting mixture was stirred at 80-90° C. until clear solution formed, then piperidine-4-carboxylic acid (15 g, 116.14 mmol) and 2-amino-4-chloro-benzenethiol (18.54 g, 116.14 mmol) were added in one portion under argon and the resulting mixture was stirred under argon at 160° C. for 15 hr, then cooled down and diluted with water (500 ml). The resulting precipitate was filtered, transferred to a beaker and basified to pH 11 with 10% aqueous sodium hydroxide solution. The resulting mixture was extracted with DCM (2*250 ml). The combined organic extracts were dried over sodium sulphate, then hydrogen chloride solution 4.0M in dioxane (157.50 g, 600.44 mmol, 150 mL, 13.9% purity) was slowly added to a dry dichloromethane solution of the product. The resulting precipitate was filtered, washed with DCM (3*50 ml) and dried in vacuum to afford 5-chloro-2-(4-piperidyl)-1,3-benzothiazole (22 g, 67.55 mmol, 58.16% yield, 2HCl) as green solid, which was used directly in the next step. LCMS(ESI): [M]⁺ m/z: calcd 252.2; found 253.2; Rt=0.963 min.

### Step 2: Synthesis of 5-chloro-2-(1-(oxetan-3-yl)piperidin-4-yl)benzo[d]thiazole

Oxetan-3-one (1.33 g, 18.42 mmol, 1.19 mL) was added in one portion at 25° C. to a stirred mixture of 5-chloro-2-(4-piperidyl)-1,3-benzothiazole (4 g, 12.28 mmol, 2HCl) and sodium acetate, anhydrous (2.52 g, 30.70 mmol, 1.65 mL) in MeOH (100 mL). The resulting mixture was stirred at 25° C. for 2 hr, then sodium cyanoborohydride (1.54 g, 24.56 mmol) was added in one portion. The reaction mixture was stirred at 25° C. for 15 hr, then concentrated to dryness in vacuum. The residue was diluted with water (40 ml) and basified to pH 10-11 with 10% aqueous sodium hydroxide solution. The resulting mixture was extracted with DCM (2*75 ml). The combined organic extracts were dried over sodium sulphate and concentrated in vacuum to afford 5-chloro-2-[1-(oxetan-3-yl)-4-piperidyl]-1,3-benzothiazole (3.5 g, 11.33 mmol, 92.28% yield) as green solid, which was used directly in the next step. LCMS(ESI): [M]⁺ m/z: calcd 308.2; found 309.2; Rt=1.776 min.

### Step 3: Synthesis of 2-(1-(oxetan-3-yl)piperidin-4-yl)-5-(4,4,5,5-tetramethyl-1,3,2-dioxaborolan-2-yl)benzo[d]thiazole

A mixture of 5-chloro-2-[1-(oxetan-3-yl)-4-piperidyl]-1,3-benzothiazole (3.5 g, 11.33 mmol), 4,4,5,5-tetramethyl-2-(4,4,5,5-tetramethyl-1,3,2-dioxaborolan-2-yl)-1,3,2-dioxaborolane (3.17 g, 12.47 mmol) and potassium acetate (2.22 g, 22.67 mmol, 1.42 mL) in dioxane (100 mL) was evacuated and then backfilled with argon. This operation was repeated two times, then tris(1,5-diphenylpenta-1,4-dien-3-one)dipalladium(518.90 mg, 566.66 μmol) and dicyclohexyl[2′,4′,6′-tris(propan-2-yl)-[1,1′-biphenyl]-2-yl]phosphane (1.08 g, 2.27 mmol) were added under argon, and the reaction mixture was stirred at 95° C. for 36 hr. The reaction mixture was cooled down and concentrated in vacuum. The residue was purified by column chromatography on silica gel using chloroform/MeCN gradient (0-100% MeCN) to afford 2-[1-(oxetan-3-yl)-4-piperidyl]-5-(4,4,5,5-tetramethyl-1,3,2-dioxaborolan-2-yl)-1,3-benzothiazole (2.6 g, 6.49 mmol, 57.30% yield) as green solid. LCMS(ESI): [M]⁺ m/z: calcd 400.2; found 401.2; Rt=1.113 min.

### Step 4: Synthesis of (S)-tert-butyl 3-methyl-6-(2-(1-(oxetan-3-yl)piperidin-4-yl)benzo[d]thiazol-5-yl)-3,4-dihydropyridine-1(2H)-carboxylate

Prepared by general procedure scheme S2 step 3. Yield: 2.7 g (88.52%).

CC conditions: The crude product was purified by silica gel with CHCl₃/MeCN gradient (0-100% MeCN) as an eluent mixture.

LCMS(ESI): [M]⁺ m/z: calcd 469.2; found 470.2; Rt=1.233 min.

### Step 5: Synthesis of (S)-5-(5-methyl-3,4,5,6-tetrahydropyridin-2-yl)-2-(1-(oxetan-3-yl)piperidin-4-yl)benzo[d]thiazole

Prepared by general procedure scheme S2 step 4. Yield: 900 mg of crude.

LCMS(ESI): [M]⁺ m/z: calcd 369.2; found 370.2; Rt=0.484 min.

### Step 6: Synthesis of 5-((2R,5S)-5-methylpiperidin-2-yl)-2-(1-(oxetan-3-yl)piperidin-4-yl)benzo[d]thiazole

Prepared by general procedure scheme S2 step 5. Yield: 650 mg (71.83%).

LCMS(ESI): [M]⁺ m/z: calcd 371.2; found 372.2; Rt=0.625 min.

### Step 7: Synthesis of 2,2,2-trifluoroethyl 2-((2R,5S)-5-methyl-2-(2-(1-(oxetan-3-yl)piperidin-4-yl)benzo[d]thiazol-5-yl)piperidin-1-yl)-2-oxoacetate

Prepared by general procedure Scheme S3 step 1. Yield: 475 mg of crude.

LCMS(ESI): [M]⁺ m/z: calcd 525.2; found 526.2; Rt=1.144 min.

### Step 8: Synthesis of 2-((2R,5S)-5-methyl-2-(2-(1-(oxetan-3-yl)piperidin-4-yl)benzo[d]thiazol-5-yl)piperidin-1-yl)-2-oxoacetamide

2,2,2-Trifluoroethyl 2-[(2R,5S)-5-methyl-2-[2-[1-(oxetan-3-yl)-4-piperidyl]-1,3-benzothiazol-5-yl]-1-piperidyl]-2-oxo-acetate (475 mg, 903.76 μmol) was dissolved in ammonia (7N in MeOH) (3.90 g, 34.31 mmol, 5 mL, 15% purity). Resulting mixture was stirred at 20° C. for 16 hr. Then, volatiles were removed under reduced pressure, leaving 2-[(2R,5S)-5-methyl-2-[2-[1-(oxetan-3-yl)-4-piperidyl]-1,3-benzothiazol-5-yl]-1-piperidyl]-2-oxo-acetamide (325 mg, 734.34 μmol, 81.25% yield). LCMS(ESI): [M]⁺ m/z: calcd 442.2; found 443.2; Rt=0.725 min.

### Intermediate 17. N,N-dimethyl-1-(5-((2R,5S)-5-methylpiperidin-2-yl)benzo[d]thiazol-2-yl)methanamine

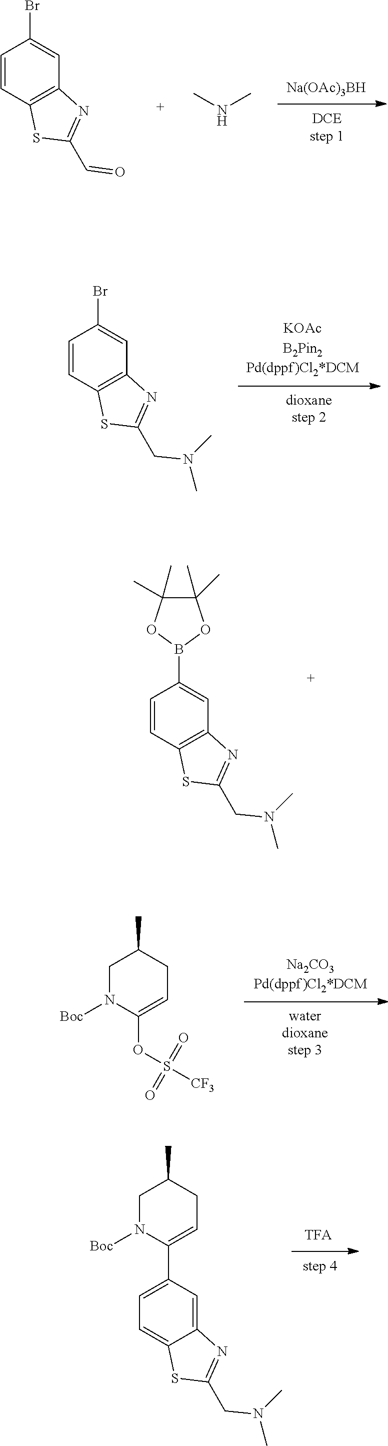

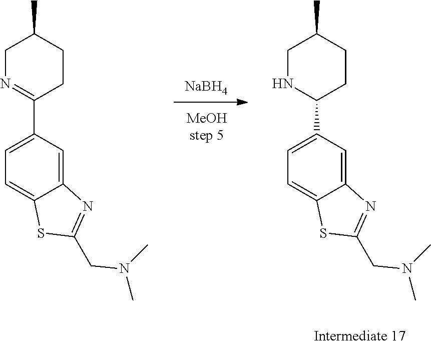 [SMILES: C[C@H]1CCC(c2ccc3sc(CN(C)C)nc3c2)=NC1 ; "NaBH4" ; C[C@H]1CC[C@H](c2ccc3sc(CN(C)C)nc3c2)NC1]

### Step 1: Synthesis of 1-(5-bromobenzo[d]thiazol-2-yl)-N,N-dimethylmethanamine

Prepared by general procedure scheme S2 step 1C. Yield: 3.7 g (82.58%).

LCMS(ESI): [M]⁺ m/z: calcd 271.2; found 272.2; Rt=0.801 min.

### Step 2: Synthesis of N,N-dimethyl-1-(5-(4,4,5,5-tetramethyl-1,3,2-dioxaborolan-2-yl)benzo[d]thiazol-2-yl)methanamine

Prepared by general procedure scheme S2 step 2. Yield: 12.91 g (70.07%).

CC conditions: The crude product was purified by silica gel with DCM/MeCN (gradient) as an eluent mixture.

LCMS(ESI): [M]⁺ m/z: calcd 318.2; found 319.2; Rt=0.758 min.

### Step 3: Synthesis of (S)-tert-butyl 6-(2-((dimethylamino)methyl)benzo[d]thiazol-5-yl)-3-methyl-3,4-dihydropyridine-1(2H)-carboxylate

Prepared by general procedure scheme S2 step 3. Yield: 3.5 g of crude.

LCMS(ESI): [M]⁺ m/z: calcd 387.2; found 388.2; Rt=1.192 min.

### Step 4: Synthesis of (S)—N,N-dimethyl-1-(5-(5-methyl-3,4,5,6-tetrahydropyridin-2-yl)benzo[d]thiazol-2-yl)methanamine

Prepared by general procedure scheme S2 step 4. Yield: 3.04 g of crude.

LCMS(ESI): [M]⁺ m/z: calcd 287.2; found 288.2; Rt=0.602 min.

### Step 5: Synthesis of N,N-dimethyl-1-(5-((2R,5S)-5-methylpiperidin-2-yl)benzo[d]thiazol-2-yl)methanamine

Prepared by general procedure scheme S2 step 5. Yield: 2.5 g of crude.

LCMS(ESI): [M]⁺ m/z: calcd 289.2; found 290.2; Rt=0.686 min.

### Intermediate 18. 1-methyl-4-(5-((2R,5S)-5-methylpiperidin-2-yl)benzo[d]thiazol-2-yl)piperidin-2-one

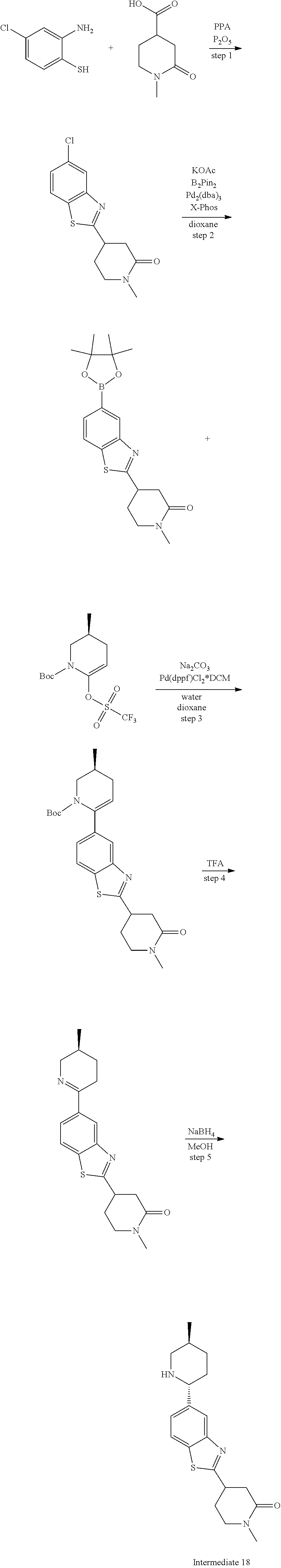

### Step 1: Synthesis of 4-(5-chlorobenzo[d]thiazol-2-yl)-1-methylpiperidin-2-one

Prepared by general procedure scheme S2 step 1A. Yield: 3.1 g (35.25%).

CC conditions: The crude product was purified by silica gel with CHCl₃/MTBE (gradient 10-100% MTBE) as an eluent mixture.

LCMS(ESI): [M]⁺ m/z: calcd 280.2; found 281.2; Rt=1.118 min.

### Step 2: Synthesis of 1-methyl-4-(5-(4,4,5,5-tetramethyl-1,3,2-dioxaborolan-2-yl)benzo[d]thiazol-2-yl)piperidin-2-one

tris(Dibenzylideneacetone)dipalladium(0) (303.31 mg, 331.23 μmol) and XPhos (631.61 mg, 1.32 mmol) was added to a solution of 4-(5-chloro-1,3-benzothiazol-2-yl)-1-methyl-piperidin-2-one (3.1 g, 11.04 mmol) and 4,4,5,5-tetramethyl-2-(4,4,5,5-tetramethyl-1,3,2-dioxaborolan-2-yl)-1,3,2-dioxaborolane (3.64 g, 14.35 mmol) in dioxane (100 mL).

Reaction flask was evacuated and refilled with argon 3 times. Then potassium acetate (2.17 g, 22.08 mmol, 1.38 mL) was added under stream of argon. Resulting mixture was stirred at 100° C. for 12 hr under inert atmosphere, then cooled and evaporated in vacuum poured into water (200 ml) and extracted with DCM (2×100 ml), dried over sodium sulphate and evaporated in vacuum to leave 5 g of crude product, 5 g of which was purification by column chromatography on silica gel using CHCl₃/MeCN gradient (10-100% MeCN) to afford 1-methyl-4-[5-(4,4,5,5-tetramethyl-1,3,2-dioxaborolan-2-yl)-1,3-benzothiazol-2-yl]piperidin-2-one (3 g, 8.06 mmol, 72.99% yield). LCMS(ESI): [M]⁺ m/z: calcd 372.2; found 373.2; Rt=1.299 min.

### Step 3: Synthesis of (3S)-tert-butyl 3-methyl-6-(2-(1-methyl-2-oxopiperidin-4-yl)benzo[d]thiazol-5-yl)-3,4-dihydropyridine-1(2H)-carboxylate

Prepared by general procedure scheme S2 step 3. Yield: 3 g of crude.

LCMS(ESI): [M]⁺ m/z: calcd 441.2; found 442.2; Rt=4.017 min.

### Step 4: Synthesis of 1-methyl-4-(5-((S)-5-methyl-3,4,5,6-tetrahydropyridin-2-yl)benzo[d]thiazol-2-yl)piperidin-2-one

Prepared by general procedure scheme S2 step 4. Yield: 1.1 g of crude.

LCMS(ESI): [M]⁺ m/z: calcd 341.2; found 342.2; Rt=0.860 min.

### Step 5: Synthesis of 1-methyl-4-(5-((2R,5S)-5-methylpiperidin-2-yl)benzo[d]thiazol-2-yl)piperidin-2-one

Prepared by general procedure scheme S2 step 5. Yield: 0.7 g of crude.

LCMS(ESI): [M]⁺ m/z: calcd 343.2; found 344.2; Rt=0.706 min.

### Intermediate 19. 2-[(2R,5S)-5-methyl-2-[2-(1-methyl-4-piperidyl)-1,3-benzothiazol-5-yl]-1-piperidyl]-2-oxo-acetamide

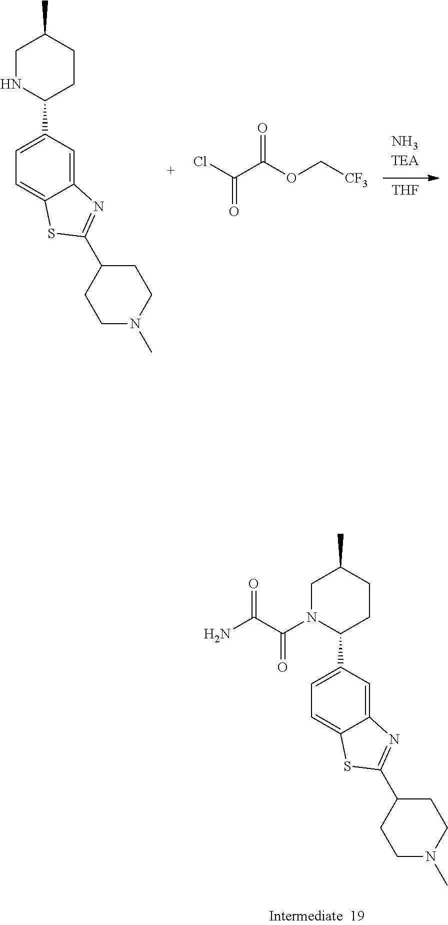 [SMILES: C[C@H]1CC[C@H](c2ccc3sc(C4CCN(C)CC4)nc3c2)NC1 ; O=C(Cl)C(=O)OCC(F)(F)F ; C[C@H]1CC[C@H](c2ccc3sc(C4CCN(C)CC4)nc3c2)N(C(=O)C(N)=O)C1 ; "THF"]

2,2,2-Trifluoroethyl 2-chloro-2-oxo-acetate (1.04 g, 5.46 mmol) was slowly added to a solution of 2-(1-methyl-4-piperidyl)-5-[(2R,5S)-5-methyl-2-piperidyl]-1,3-stirred benzothiazole (1.5 g, 4.55 mmol) and TEA (921.30 mg, 9.10 mmol, 1.27 mL) in dry THF (50 mL) at 25° C. The resulting mixture was stirred at 25° C. for 0.5 hr. Then gaseous ammonia was bubbled through the reaction mixture at 25° C. for 0.5 hr. The resulting ammonium chloride precipitate was filtered and discarded, the filtrate was concentrated in vacuum to afford crude 2-[(2R,5S)-5-methyl-2-[2-(1-methyl-4-piperidyl)-1,3-benzothiazol-5-yl]-1-piperidyl]-2-oxo-acetamide (1.6 g, 3.99 mmol, 87.75% yield) as yellow solid, which was used directly in the next step. LCMS(ESI): [M]⁺ m/z: calcd 400.2; found 401.2; Rt=0.867 min.

### Intermediate 20. tert-butyl (2R,5S)-5-methyl-2-[2-[(3S)-1-methyl-3-piperidyl]-1,3-benzothiazol-5-yl]piperidine-1-carboxylate

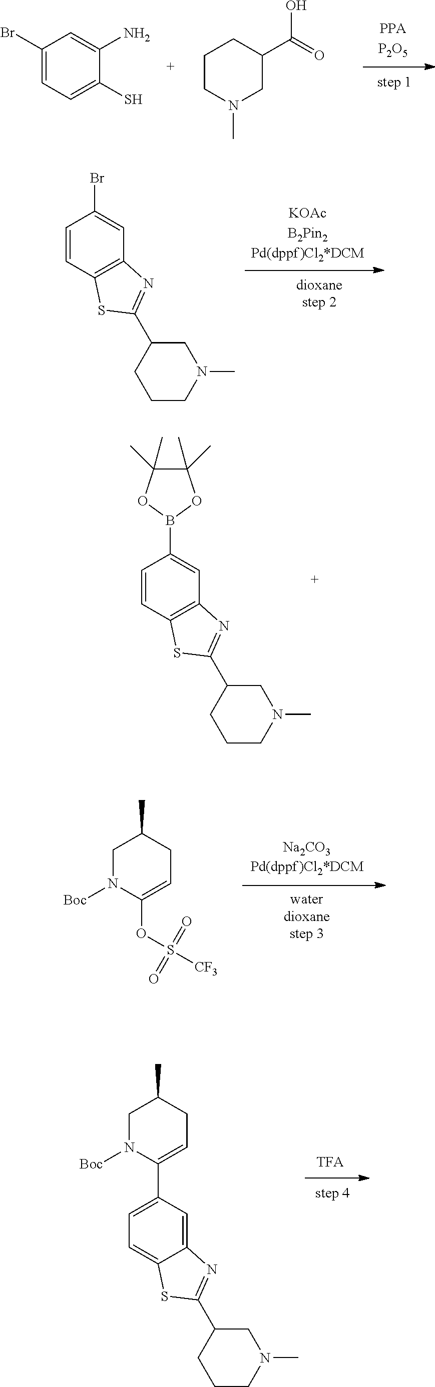

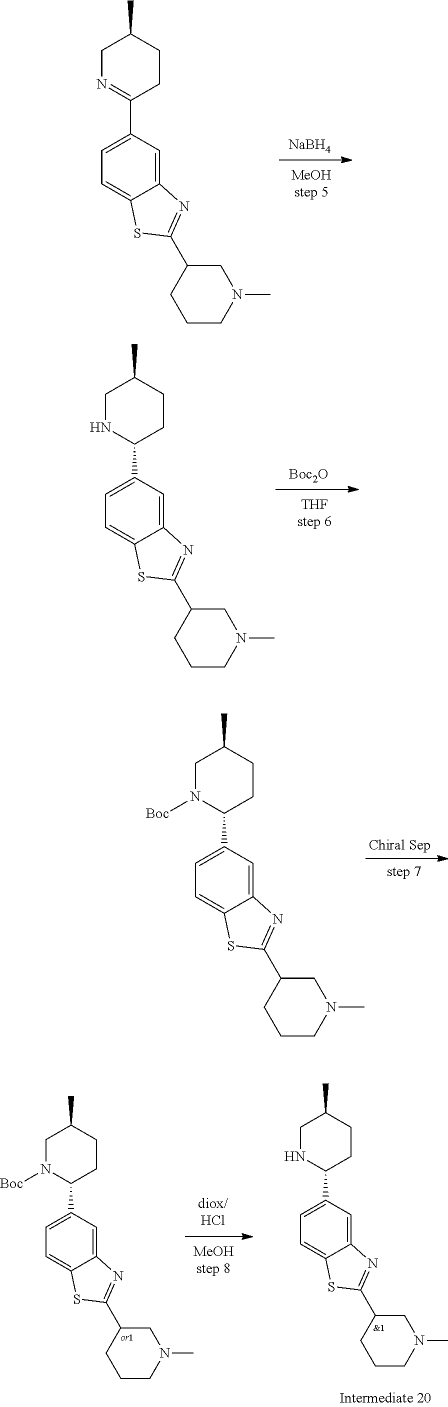

### Step 1: Synthesis of 5-bromo-2-(1-methylpiperidin-3-yl)benzo[d]thiazole

Prepared by general procedure scheme S2 step 1A. Yield: 4.3 g (93.99%).

LCMS(ESI): [M]⁺ m/z: calcd 311.2; found 312.2; Rt=0.990 min.

### Step 2: Synthesis of 2-(1-methylpiperidin-3-yl)-5-(4,4,5,5-tetramethyl-1,3,2-dioxaborolan-2-yl)benzo[d]thiazole

Prepared by general procedure scheme S2 step 2. Yield: 4.9 g of crude.

LCMS(ESI): [M]⁺ m/z: calcd 358.2; found 359.2; Rt=0.965 min.

### Step 3: Synthesis of (3S)-tert-butyl 3-methyl-6-(2-(1-methylpiperidin-3-yl)benzo[d]thiazol-5-yl)-3,4-dihydropyridine-1(2H)-carboxylate

Prepared by general procedure scheme S2 step 3. Yield: 5.8 g of crude.

LCMS(ESI): [M]⁺ m/z: calcd 427.2; found 428.2; Rt=1.150 min.

### Step 4: Synthesis of 5-((S)-5-methyl-3,4,5,6-tetrahydropyridin-2-yl)-2-(1-methylpiperidin-3-yl)benzo[d]thiazole

Prepared by general procedure scheme S2 step 4. Yield: 3 g of crude.

LCMS(ESI): [M]⁺ m/z: calcd 327.2; found 328.2; Rt=0.656 min.

### Step 5: Synthesis of 5-((2R,5S)-5-methylpiperidin-2-yl)-2-(1-methylpiperidin-3-yl)benzo[d]thiazole

Prepared by general procedure scheme S2 step 5. Yield: 1.8 g (59.63%).

LCMS(ESI): [M]⁺ m/z: calcd 329.2; found 330.2; Rt=0.491 min.

### Step 6: Synthesis of (2R,5S)-tert-butyl 5-methyl-2-(2-(1-methylpiperidin-3-yl)benzo[d]thiazol-5-yl)piperidine-1-carboxylate

To a solution of 2-(1-methyl-3-piperidyl)-5-[(2R,5S)-5-methyl-2-piperidyl]-1,3-benzothiazole (1.5 g, 4.55 mmol) in THF (4 mL), di-tert-butyl dicarbonate (993.53 mg, 4.55 mmol, 1.04 mL) was added. The resulting mixture was stirred at 25° C. for 3 hr and purified by HPLC (Device (Mobile Phase, SYSTEM Column): 55-95% 0-5 min H₂O/MeCN/0.1% NH₄OH, flow: 30 ml/min (loading pump 4 ml/min MeCN) target mass 429 column: XBridge BEH C18 5 um 130 A) to obtain tert-butyl (2R,5S)-5-methyl-2-[2-(1-methyl-3-piperidyl)-1,3-benzothiazol-5-yl]piperidine-1-carboxylate (0.99 g, 2.30 mmol, 50.62% yield). LCMS(ESI): [M]⁺ m/z: calcd 429.2; found 430.2; Rt=3.390 min.

### Step 7: Chiral Separation

Racemic tert-butyl (2R,5S)-5-methyl-2-[2-(1-methyl-3-piperidyl)-1,3-benzothiazol-5-yl]piperidine-1-carboxylate (0.99 g, 2.30 mmol) was chiral separated (Column: Chiralpak AD-H—III (250*20 mm, 5 mkm), Hexane-IPA-MeOH, 80-10-10, 12 ml/min) to obtain tert-butyl (2R,5S)-5-methyl-2-[2-[(3S)-1-methyl-3-piperidyl]-1,3-benzothiazol-5-yl]piperidine-1-carboxylate (384 mg, 893.82 μmol, 77.58% yield).

Rel Time for this isomer in analytical conditions (column: AD-H, Hexane-IPA-MeOH, 80-10-10, 0.8 ml/min as mobile phase) 11.83 min.

Retention time: 11.83 min.

LCMS(ESI): [M]⁺ m/z: calcd 429.2; found 430.2; Rt=1.099 min.

### Intermediate 21. (S)—N,N-dimethyl-3-(5-(5-methyl-3,4,5,6-tetrahydropyridin-2-yl)benzo[d]thiazol-2-yl) propan-1-amine

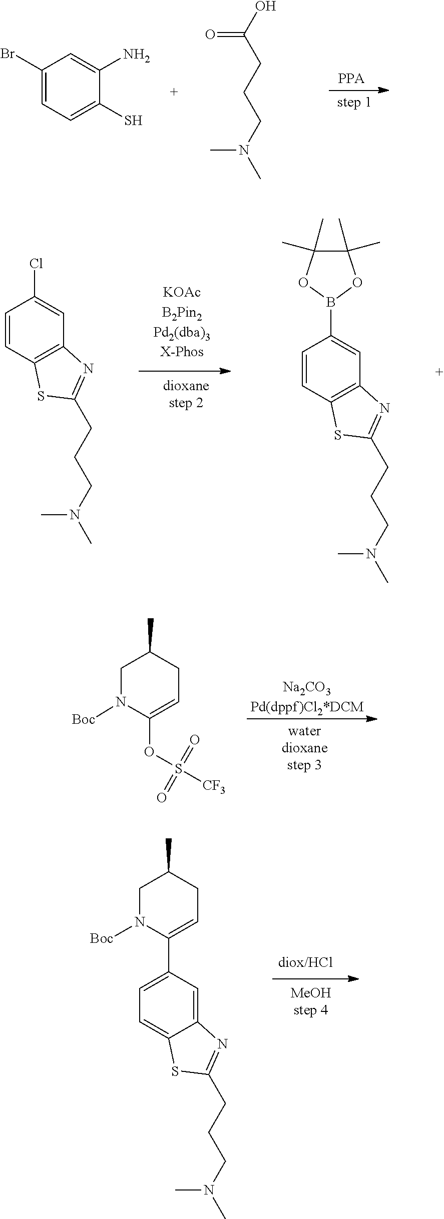

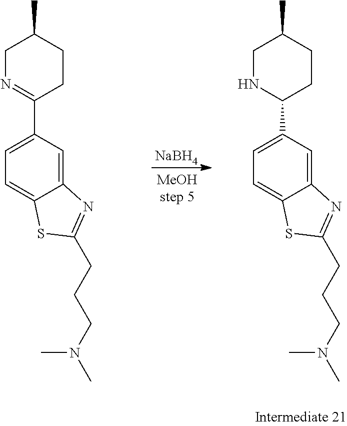 [SMILES: C[C@H]1CCC(c2ccc3sc(CCCN(C)C)nc3c2)=NC1 ; "NaBH4" ; C[C@H]1CC[C@H](c2ccc3sc(CCCN(C)C)nc3c2)NC1]

### Step 1: Synthesis of 3-(5-chlorobenzo[d]thiazol-2-yl)-N,N-dimethylpropan-1-amine

The stirred solution of 2-amino-4-chloro-benzenethiol (1 g, 6.26 mmol) and 4-(dimethylamino) butanoic acid (1.3 g, 7.76 mmol, HCl) in PPA (5 mL) was allowed to stir at 120° C. for 16 hr. Upon completion, the reaction mixture was quenched with water (100 mL) and neutralized by NaOH to pH=8. The aqueous phase was extracted with EtOAc (2*20 mL). The combined organic phase was dried over Na₂SO₄ and concentrated under reduced pressure. The desired product 3-(5-chloro-1,3-benzothiazol-2-yl)-N,N-dimethyl-propan-1-amine (1.2 g, 4.71 mmol, 75.19% yield) was isolated. LCMS(ESI): [M]⁺ m/z: calcd 254.2; found 255.2; Rt=0.909 min.

### Step 2: Synthesis of N,N-dimethyl-3-(5-(4,4,5,5-tetramethyl-1,3,2-dioxaborolan-2-yl)benzo[d]thiazol-2-yl) propan-1-amine

To a stirred solution of 3-(5-chloro-1,3-benzothiazol-2-yl)-N,N-dimethyl-propan-1-amine (0.6 g, 2.35 mmol) and 4,4,5,5-tetramethyl-2-(4,4,5,5-tetramethyl-1,3,2-dioxaborolan-2-yl)-1,3,2-dioxaborolane (0.6 g, 2.36 mmol) in dioxane (10 mL) were added Pd₂dba₃ (0.4 g, 436.82 μmol) and XPhos (0.4 g, 839.10 μmol). The resulting suspension was degassed with argon at 50° C. for 0.5 hr. Potassium acetate (0.5 g, 5.09 mmol, 318.47 μL) was added. The reaction mixture was stirred at 100° C. for 16 hr. Upon completion, the reaction mixture was concentrated under reduced pressure, quenched with water (20 mL), the aqueous phase was extracted with CHCl₃ (2*20 mL). The organic phase was extracted with 10% HCl (2*40 ml). The aqueous phase was neutralized by NaHCO₃ to pH=8, extracted with CHCl₃ (2*20 mL). The organic phase was dried over Na₂SO₄ and evaporated in vacuum. The desired product N,N-dimethyl-3-[5-(4,4,5,5-tetramethyl-1,3,2-dioxaborolan-2-yl)-1,3-benzothiazol-2-yl]propan-1-amine (0.8 g, 2.31 mmol, 98.10% yield) was isolated. LCMS(ESI): [M]⁺ m/z: calcd 346.2; found 347.2; Rt=0.940 min.

### Step 3: Synthesis of (S)-tert-butyl 6-(2-(3-(dimethylamino) propyl)benzo[d]thiazol-5-yl)-3-methyl-3,4-dihydropyridine-1(2H)-carboxylate

Prepared by general procedure scheme S2 step 3. Yield: 0.4 g of crude.

CC conditions: The crude product was purified by silica gel with EtOAc/MeOH as an eluent mixture.

LCMS(ESI): [M]⁺ m/z: calcd 415.2; found 416.2; Rt=1.033 min.

### Step 4: Synthesis of (S)—N,N-dimethyl-3-(5-(5-methyl-3,4,5,6-tetrahydropyridin-2-yl)benzo[d]thiazol-2-yl) propan-1-amine

The stirred solution of tert-butyl (3S)-6-[2-[3-(dimethylamino) propyl]-1,3-benzothiazol-5-yl]-3-methyl-3,4-dihydro-2H-pyridine-1-carboxylate (400.00 mg, 962.49 μmol) in MeOH (10 mL) and diox/HCl (10 mL) was allowed to stir at 25° C. for 16 hr. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The desired product N,N-dimethyl-3-[5-[(3S)-3-methyl-2,3,4,5-tetrahydropyridin-6-yl]-1,3-benzothiazol-2-yl]propan-1-amine (0.3 g, 950.95 μmol, 98.80% yield) was isolated. LCMS(ESI): [M]⁺ m/z: calcd 315.2; found 316.2; Rt=0.671 min.

### Step 5: Synthesis of N,N-dimethyl-3-(5-((2R,5S)-5-methylpiperidin-2-yl)benzo[d]thiazol-2-yl) propan-1-amine

Prepared by general procedure scheme S2 step 5. Yield: 0.3 g of crude.

LCMS(ESI): [M]⁺ m/z: calcd 317.2; found 318.2; Rt=0.754 min.

### Intermediate 22. 2-(1-methyl-4-piperidyl)-7-[(2R,5S)-5-methyl-2-piperidyl]quinoline

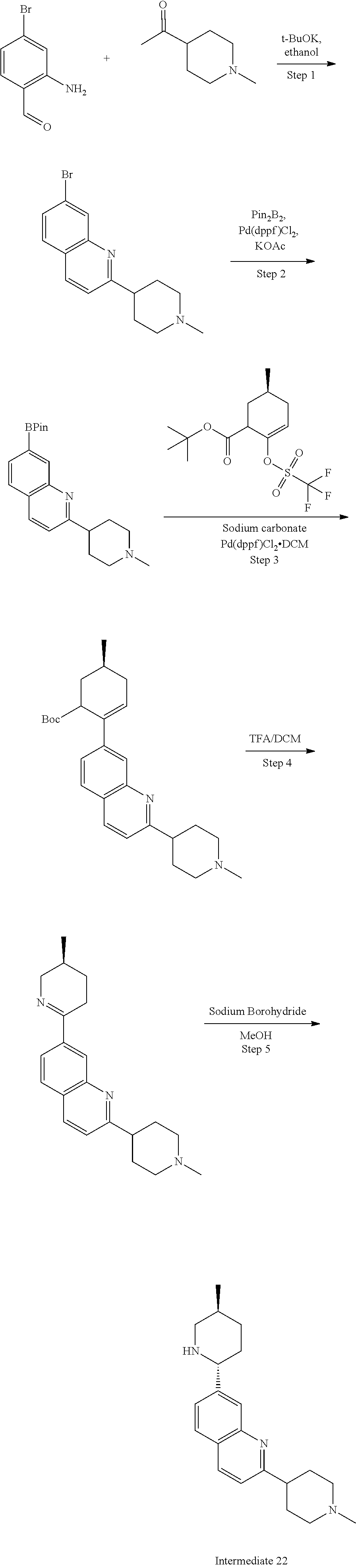

### Step 1:7-bromo-2-(1-methyl-4-piperidyl)quinoline

2-amino-4-bromo-benzaldehyde (1.3 g, 6.50 mmol), 1-(1-methyl-4-piperidyl) ethanone (917.72 mg, 6.50 mmol) and Sodium tert-butoxide (1.25 g, 13.00 mmol) were mixed in ethanol (20 mL) and stirred for 12 hr at 80° C. The RM was concentrated in vacuo, then treated with DCM, washed with water. Organic phase was dried over Na2SO4 and evaporated to give 7-bromo-2-(1-methyl-4-piperidyl)quinoline (1.65 g, crude). LCMS(ESI): [M+1]⁺ m/z: calcd 304.1; found 305; Rt=0.934 min.

### Step 2:2-(1-methyl-4-piperidyl)-7-(4,4,5,5-tetramethyl-1,3,2-dioxaborolan-2-yl)quinoline

A mixture of 7-bromo-2-(1-methyl-4-piperidyl)quinoline (1.95 g, 6.39 mmol) (300 mg), Bis(pinacolato)diboron (2.11 g, 8.31 mmol) and Potassium Acetate (1.88 g, 19.17 mmol, 1.20 mL) in Dioxane (30.03 mL) was degassed with argon for 10 min. Pd(dppf)Cl₂*DCM (521.75 mg, 638.90 μmol) was next added and the reaction mixture was heated at 80° C. for 12 hr. The reaction mixture was filtered, then concentrated in vacuo. The residue was treated with mixture of MTBE-Hex (1:1), filtered and then concentrated under reduced pressure to give 2-(1-methyl-4-piperidyl)-7-(4,4,5,5-tetramethyl-1,3,2-dioxaborolan-2-yl)quinoline (3 g, crude).

LCMS(ESI): [M+1]⁺ m/z: calcd 352.3; found 353.2; Rt=1.119 min.

### Step 3: tert-butyl (3S)-3-methyl-6-[2-(1-methyl-4-piperidyl)-7-quinolyl]-3,4-dihydro-2H-pyridine-1-carboxylate

2-(1-methyl-4-piperidyl)-7-(4,4,5,5-tetramethyl-1,3,2-dioxaborolan-2-yl)quinoline (2.8 g, 7.95 mmol), Sodium carbonate (2.53 g, 23.84 mmol, 998.15 μL), tert-butyl (3S)-3-methyl-6-(trifluoromethylsulfonyloxy)-3,4-dihydro-2H-pyridine-1-carboxylate (2.74 g, 7.95 mmol) and Pd(dppf)Cl₂*DCM (0.5 g, 612.75 μmol) were mixed in H2O (15 mL) and dioxane (50 mL) under argon and stirred at 75° C. for 12 hr. Reaction mixture was diluted with water and desired product extracted with DCM, dried over Na2SO4 and concentrated in vacuo. tert-butyl (3S)-3-methyl-6-[2-(1-methyl-4-piperidyl)-7-quinolyl]-3,4-dihydro-2H-pyridine-1-carboxylate (4 g, crude) was obtained. LCMS(ESI): [M+1]⁺ m/z: calcd 421.3; found 422.2; Rt=1.174 min.

### Step 4:2-(1-methyl-4-piperidyl)-7-[(3S)-3-methyl-2,3,4,5-tetrahydropyridin-6-yl]quinoline

tert-butyl (3S)-3-methyl-6-[2-(1-methyl-4-piperidyl)-7-quinolyl]-3,4-dihydro-2H-pyridine-1-carboxylate (4 g, 9.49 mmol) was dissolved in DCM (40 mL) and CF₃COOH (15 g, 9.49 mmol) was added. The RM was stirred for 2 hr, then concentrated. The residue was treated with MTBE two times. Black gum was treated with aq. solution of NaHCO₃ and then extracted with DCM. Organic phase was dried over Na₂SO₄ and then concentrated in vacuo to give 2-(1-methyl-4-piperidyl)-7-[(3S)-3-methyl-2,3,4,5-tetrahydropyridin-6-yl]quinoline (1 g, crude). LCMS(ESI): [M+1]⁺ m/z: calcd 321.2; found 322.2; Rt=0.682 min.

### Step 5:2-(1-methyl-4-piperidyl)-7-[(2R,5S)-5-methyl-2-piperidyl]quinoline

To a solution of 2-(1-methyl-4-piperidyl)-7-[(3S)-3-methyl-2,3,4,5-tetrahydropyridin-6-yl]quinoline (1 g, 2.49 mmol) in methanol (30 mL) Sodium Borohydride (188.30 mg, 4.98 mmol, 175.33 μL) was added, then stirred overnight. The RM was concentrated in vacuo, then treated with DCM, filtered and evaporated to give 2-(1-methyl-4-piperidyl)-7-[(2R,5S)-5-methyl-2-piperidyl]quinoline (0.75 g, crude). LCMS(ESI): [M+1]⁺ m/z: calcd 323.2; found 324.2; Rt=0.705 min.

### Intermediate 23. 2-((2R,5S)-5-methyl-2-(2-(2-(pyrrolidin-1-yl)ethyl)benzo[d]thiazol-5-yl)piperidin-1-yl)-2-oxoacetamide

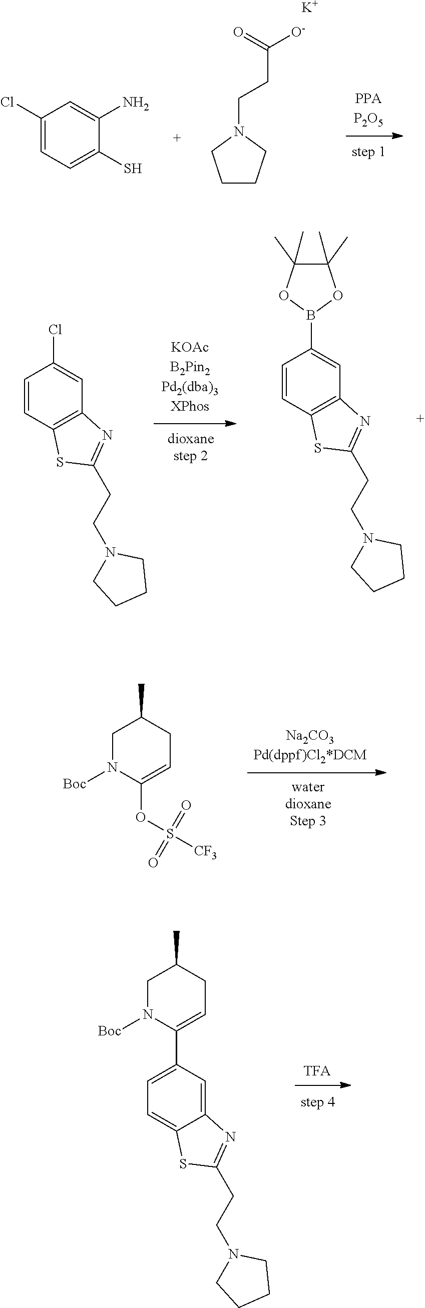

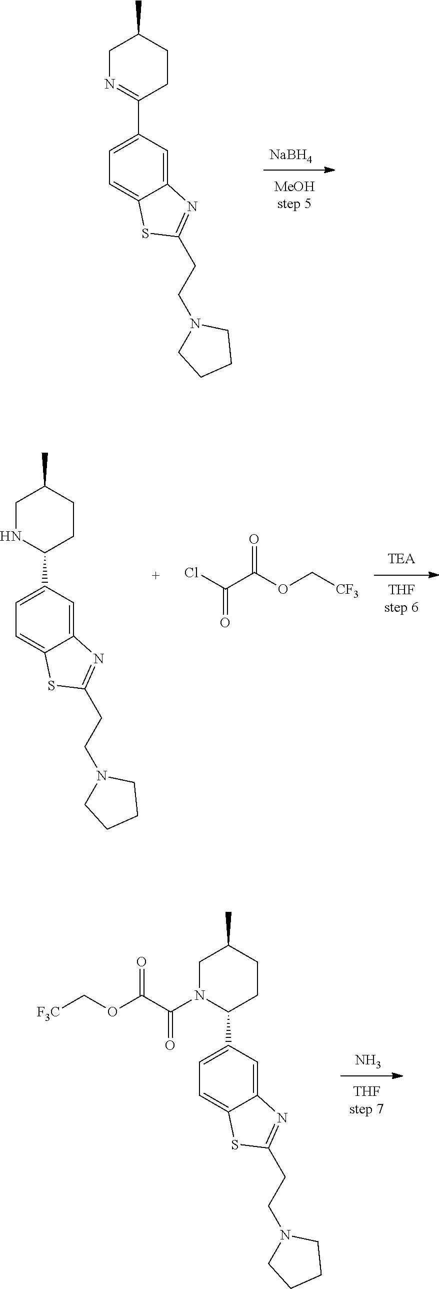

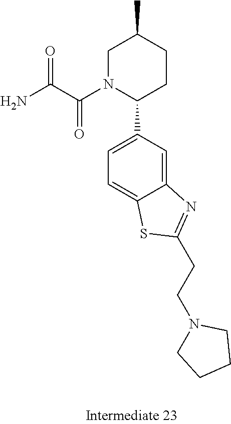 [SMILES: C[C@H]1CC[C@H](c2ccc3sc(CCN4CCCC4)nc3c2)N(C(=O)C(N)=O)C1]

### Step 1: Synthesis of 5-chloro-2-(2-(pyrrolidin-1-yl)ethyl)benzo[d]thiazole

Prepared by general procedure scheme S2 step 1A. Yield: 4.3 g (51.46%).

CC conditions: The crude product was purified by silica gel with MTBE/MeOH as an eluent mixture.

LCMS(ESI): [M]⁺ m/z: calcd 266.2; found 267.2; Rt=1.014 min.

### Step 2: Synthesis of 2-(2-(pyrrolidin-1-yl)ethyl)-5-(4,4,5,5-tetramethyl-1,3,2-dioxaborolan-2-yl)benzo[d]thiazole

Potassium acetate (3.16 g, 32.23 mmol, 2.01 mL) was added to a solution of 5-chloro-2-(2-pyrrolidin-1-ylethyl)-1,3-benzothiazole (4.30 g, 16.12 mmol) and 4,4,5,5-tetramethyl-2-(4,4,5,5-tetramethyl-1,3,2-dioxaborolan-2-yl)-1,3,2-dioxaborolane (4.50 g, 17.73 mmol) in dioxane (50 mL). Reaction flask was evacuated and refilled with argon 3 times. Then tris(dibenzylideneacetone)dipalladium(0) (737.96 mg, 805.88 μmol) and XPhos (1.54 g, 3.22 mmol) was added under stream of argon. Resulting mixture was stirred at 90° C. for 18 hr under inert atmosphere. Then pyrrolidine 40% aqueous solution (0.5 eq.) was added and the reaction mixture was stirred at 60° C. another 12 hr. Then, it was cooled, diluted with EtOAc (200 mL) and washed with Na₂CO₃ (50 mL, sat. aq.). Organic layer was dried over Na₂SO₄, concentrated under reduced pressure and the residue was purified by silica gel flash chromatography eluting with a 0 to 100 percent CHCl₃-MeCN-MeOH gradient to afford product 2-(2-pyrrolidin-1-ylethyl)-5-(4,4,5,5-tetramethyl-1,3,2-dioxaborolan-2-yl)-1,3-benzothiazole (2.2 g, 6.14 mmol, 38.10% yield). LCMS(ESI): [M]⁺ m/z: calcd 358.2; found 359.2; Rt=1.001 min.

### Step 3: Synthesis of (S)-tert-butyl 3-methyl-6-(2-(2-(pyrrolidin-1-yl)ethyl)benzo[d]thiazol-5-yl)-3,4-dihydropyridine-1(2H)-carboxylate

Prepared by general procedure scheme S2 step 3. Yield: 3 g of crude.

LCMS(ESI): [M]⁺ m/z: calcd 427.2; found 428.2; Rt=1.251 min.

### Step 4: Synthesis of (S)-5-(5-methyl-3,4,5,6-tetrahydropyridin-2-yl)-2-(2-(pyrrolidin-1-yl)ethyl)benzo[d]thiazole

Prepared by general procedure scheme S2 step 4. Yield: 1.2 g of crude.

LCMS(ESI): [M]⁺ m/z: calcd 327.2; found 328.2; Rt=0.681 min.

### Step 5: Synthesis of 5-((2R,5S)-5-methylpiperidin-2-yl)-2-(2-(pyrrolidin-1-yl)ethyl)benzo[d]thiazole

Prepared by general procedure scheme S2 step 5. Yield: 1 g of crude.

LCMS(ESI): [M]⁺ m/z: calcd 329.2; found 330.2; Rt=0.720 min.

### Step 6: Synthesis of 4,4,4-trifluoro-1-((2R,5S)-5-methyl-2-(2-(2-(pyrrolidin-1-yl)ethyl)benzo[d]thiazol-5-yl)piperidin-1-yl) butane-1,2-dione

Prepared by general procedure Scheme S3 step 1. Yield: 0.86 g of crude.

LCMS(ESI): [M]⁺ m/z: calcd 483.2; found 484.2; Rt=1.182 min.

### Step 7: Synthesis of 2-((2R,5S)-5-methyl-2-(2-(2-(pyrrolidin-1-yl)ethyl)benzo[d]thiazol-5-yl)piperidin-1-yl)-2-oxoacetamide

Prepared by general procedure Scheme S3 step 2. Yield: 0.6 g of crude.

LCMS(ESI): [M]⁺ m/z: calcd 400.2; found 401.2; Rt=0.734 min.

### Intermediate 24. rac-N,N-dimethyl-2-(5-((2R,5S)-5-methylpiperidin-2-yl)benzo[d]thiazol-2-yl)ethanamine

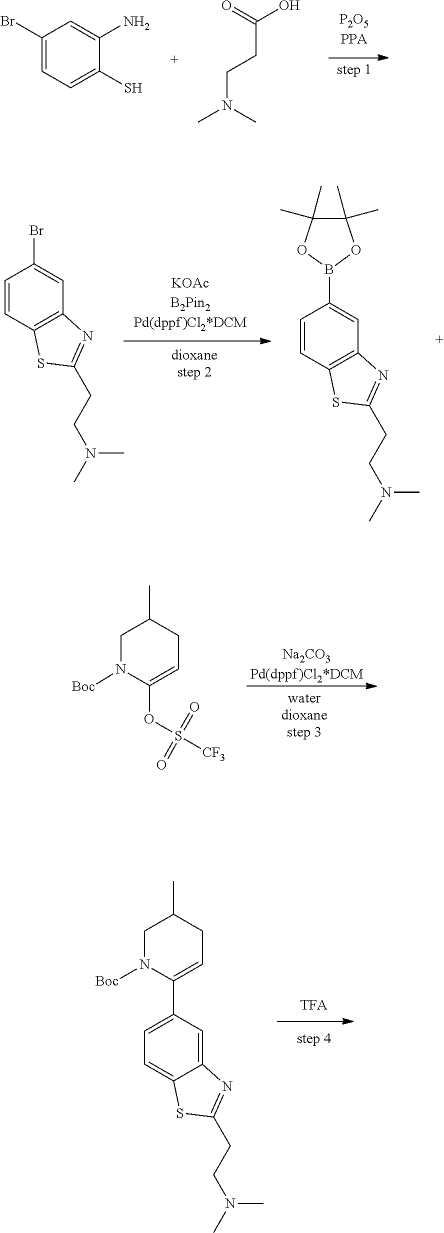

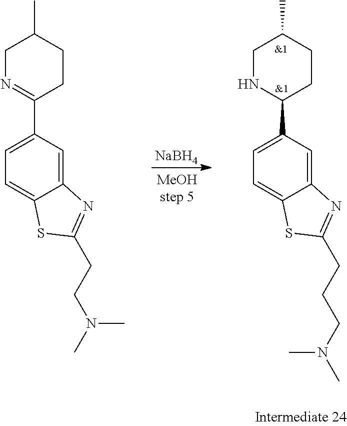 [SMILES: CC1CCC(c2ccc3sc(CCN(C)C)nc3c2)=NC1 ; "NaBH4" ; C[C@@H]1CC[C@@H](c2ccc3sc(CCCN(C)C)nc3c2)NC1]

### Step 1: Synthesis of 2-(5-bromobenzo[d]thiazol-2-yl)-N,N-dimethylethanamine

Prepared by general procedure Scheme S4 step 1A. Yield: 1 g (11.18%).

CC conditions: The crude product was purified by silica gel with CHCl₃/MeOH as an eluent mixture.

LCMS(ESI): [M]⁺ m/z: calcd 285.2; found 286.2; Rt=0.824 min.

### Step 2: Synthesis of N,N-dimethyl-2-(5-(4,4,5,5-tetramethyl-1,3,2-dioxaborolan-2-yl)benzo[d]thiazol-2-yl)ethanamine

Prepared by general procedure Scheme S4 step 2. Yield: 1.1 g of crude.

LCMS(ESI): [M]⁺ m/z: calcd 332.2; found 333.2; Rt=1.004 min.

### Step 3: Synthesis of tert-butyl 6-(2-(2-(dimethylamino)ethyl)benzo[d]thiazol-5-yl)-3-methyl-3,4-dihydropyridine-1(2H)-carboxylate

Prepared by general procedure Scheme S4 step 3. Yield: 1.5 g of crude.

LCMS(ESI): [M]⁺ m/z: calcd 401.2; found 402.2; Rt=1.064 min.

### Step 4: Synthesis of N,N-dimethyl-2-(5-(5-methyl-3,4,5,6-tetrahydropyridin-2-yl)benzo[d]thiazol-2-yl)ethanamine

Prepared by general procedure Scheme S4 step 4. Yield: 1.3 g of crude.

LCMS(ESI): [M]⁺ m/z: calcd 301.2; found 302.2; Rt=0.641 min.

### Step 5: Synthesis of rac-N,N-dimethyl-2-(5-((2R,5S)-5-methylpiperidin-2-yl)benzo[d]thiazol-2-yl)ethanamine

Prepared by general procedure scheme S2 step 5. Yield: 1.2 g of crude.

LCMS(ESI): [M]⁺ m/z: calcd 303.2; found 304.2; Rt=0.486 min.

### Intermediate 25. 2-(1-ethylpiperidin-4-yl)-5-((2R,5S)-5-methylpiperidin-2-yl)benzo[d]thiazole

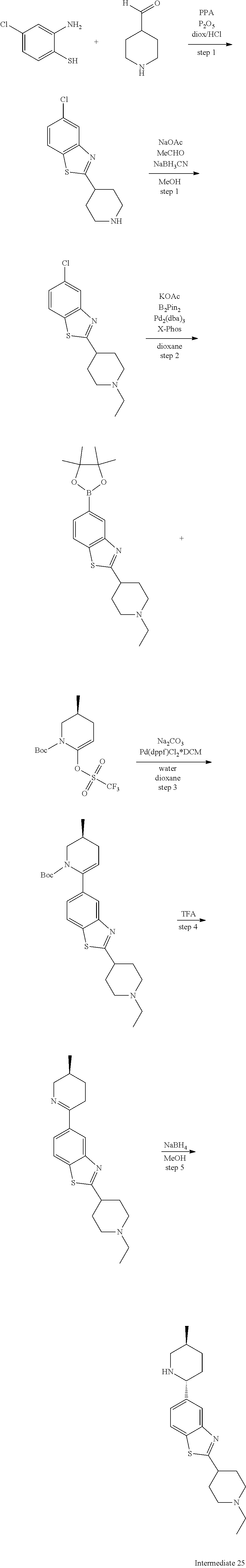

### Step 1: Synthesis of 5-chloro-2-(piperidin-4-yl)benzo[d]thiazole

Phosphorus (V) pentoxide (50 g, 352.25 mmol) was added portion wise with stirring to a phosphoric acid (60 g, 612.27 mmol, 35.29 mL). The resulting mixture was stirred at 80-90° C. until clear solution formed, then piperidine-4-carboxylic acid (15 g, 116.14 mmol) and 2-amino-4-chloro-benzenethiol (18.54 g, 116.14 mmol) were added in one portion under argon and the resulting mixture was stirred under argon at 160° C. for 15 hr, then cooled down and diluted with water (500 ml). The resulting precipitate was filtered, transferred to a beaker and basified to pH 11 with 10% aqueous sodium hydroxide solution. The resulting mixture was extracted with DCM (2*250 ml). The combined organic extracts were dried over sodium sulphate, then hydrogen chloride solution 4.0M in dioxane (157.50 g, 600.44 mmol, 150 mL, 13.9% purity) was slowly added to a dry dichloromethane solution of the product. The resulting precipitate was filtered, washed with DCM (3*50 ml) and dried in vacuum to afford 5-chloro-2-(4-piperidyl)-1,3-benzothiazole (22 g, 67.55 mmol, 58.16% yield, 2HCl) as green solid, which was used directly in the next step. LCMS(ESI): [M]⁺ m/z: calcd 252.2; found 253.2; Rt=0.963 min.

### Step 1: Synthesis of 5-chloro-2-(1-ethylpiperidin-4-yl)benzo[d]thiazole

Acetaldehyde (973.88 mg, 22.11 mmol, 790.23 μL) was added in one portion at 25° C. to a stirred mixture of 5-chloro-2-(4-piperidyl)-1,3-benzothiazole (4 g, 12.28 mmol, 2HCl) and sodium acetate, anhydrous (2.52 g, 30.70 mmol, 1.65 mL) in MeOH (100 mL). The resulting mixture was stirred at 25° C. for 2 hr, then sodium cyano borohydride (1.54 g, 24.56 mmol) was added in one portion at 25° C. The reaction mixture was stirred at 25° C. for 12 hr, LCMS showed only presence of starting material. More acetaldehyde (1.08 g, 24.56 mmol) was added, and the reaction mixture was stirred at 25° C. for 24 hr, and then concentrated in vacuum. The residue was diluted with water (40 ml) and basified to pH 10-11 with 10% aqueous sodium hydroxide solution. The resulting mixture was extracted with MTBE (2*150 ml). The combined organic extracts were dried over sodium sulphate and concentrated in vacuum to afford 5-chloro-2-(1-ethyl-4-piperidyl)-1,3-benzothiazole (2.4 g, 8.55 mmol, 69.59% yield) as light-yellow solid, which was used directly in the next step. LCMS(ESI): [M]⁺ m/z: calcd 280.2; found 281.2; Rt=2.381 min.

### Step 2: Synthesis of 2-(1-ethylpiperidin-4-yl)-5-(4,4,5,5-tetramethyl-1,3,2-dioxaborolan-2-yl)benzo[d]thiazole

A mixture of 5-chloro-2-(1-ethyl-4-piperidyl)-1,3-benzothiazole (2.4 g, 8.55 mmol), 4,4,5,5-tetramethyl-2-(4,4,5,5-tetramethyl-1,3,2-dioxaborolan-2-yl)-1,3,2-dioxaborolane (2.39 g, 9.40 mmol) and potassium acetate (1.68 g, 17.09 mmol, 1.07 mL) in dioxane (100 mL) was evacuated and then backfilled with argon. This operation was repeated two times, then tris(1,5-diphenylpenta-1,4-dien-3-one)dipalladium(391.31 mg, 427.33 μmol) and dicyclohexyl[2′,4′,6′-tris(propan-2-yl)-[1,1′-biphenyl]-2-yl]phosphane (814.86 mg, 1.71 mmol) were added under argon, and the reaction mixture was stirred at 95° C. for 18 hr. The reaction mixture was cooled down and concentrated in vacuum. The residue was purified by column chromatography on silica gel using MTBE/MeOH gradient (0-100% MeOH) to afford 2-(1-ethyl-4-piperidyl)-5-(4,4,5,5-tetramethyl-1,3,2-dioxaborolan-2-yl)-1,3-benzothiazole (1.2 g, 3.22 mmol, 37.71% yield) as light-brown solid. LCMS(ESI): [M]⁺ m/z: calcd 372.2; found 373.2; Rt=1.181 min.

### Step 3: Synthesis of (S)-tert-butyl 6-(2-(1-ethylpiperidin-4-yl)benzo[d]thiazol-5-yl)-3-methyl-3,4-dihydropyridine-1(2H)-carboxylate

Prepared by general procedure scheme S2 step 3. Yield: 1.4 g of crude.

LCMS(ESI): [M]⁺ m/z: calcd 441.2; found 442.2; Rt=2.481 min.

### Step 4: Synthesis of (S)-2-(1-ethylpiperidin-4-yl)-5-(5-methyl-3,4,5,6-tetrahydropyridin-2-yl)benzo[d]thiazole

Prepared by general procedure scheme S2 step 4. Yield: 250 mg of crude.

LCMS(ESI): [M]⁺ m/z: calcd 341.2; found 342.2; Rt=0.567 min.

### Step 5: Synthesis of 2-(1-ethylpiperidin-4-yl)-5-((2R,5S)-5-methylpiperidin-2-yl)benzo[d]thiazole

Prepared by general procedure scheme S2 step 5. Yield: 130 mg (51.69%).

LCMS(ESI): [M]⁺ m/z: calcd 343.2; found 344.2; Rt=0.610 min.

### Intermediate 26. 2-((2R,5S)-5-methyl-2-(2-(2-methyl-2-azaspiro[3.3]heptan-6-yl)benzo[d]thiazol-5-yl)piperidin-1-yl)-2-oxoacetamide

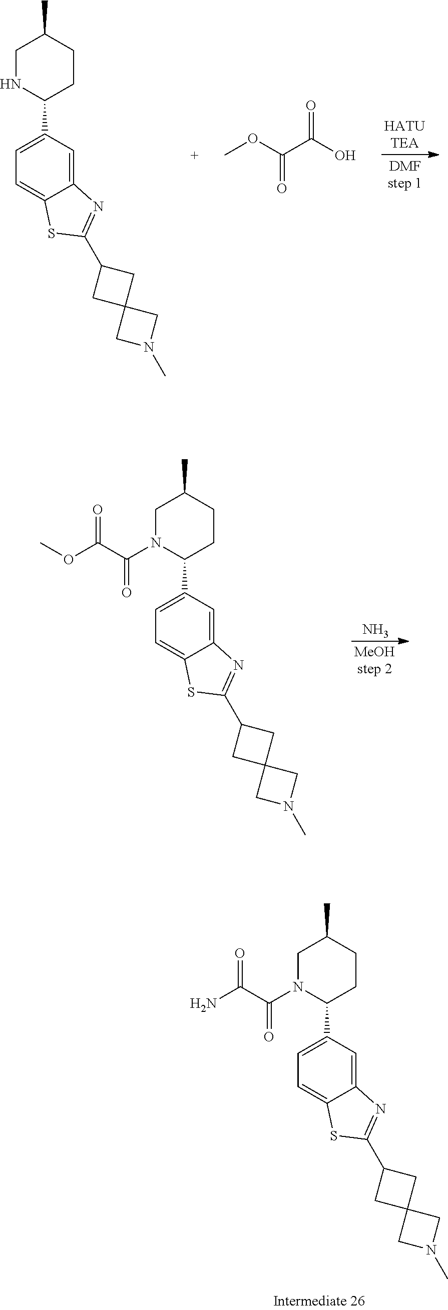

The synthesis of 2-(2-methyl-2-azaspiro[3.3]heptan-6-yl)-5-((2R,5S)-5-methylpiperidin-2-yl)benzo[d]thiazole is given in Intermediate 13.

### Step 1: Synthesis of methyl 2-((2R,5S)-5-methyl-2-(2-(2-methyl-2-azaspiro[3.3]heptan-6-yl)benzo[d]thiazol-5-yl)piperidin-1-yl)-2-oxoacetate

To a stirred mixture of 2-(2-methyl-2-azaspiro[3.3]heptan-6-yl)-5-[(2R,5S)-5-methyl-2-piperidyl]-1,3-benzothiazole (975 mg, 2.85 mmol), 2-methoxy-2-oxo-acetic acid (445.63 mg, 4.28 mmol) and TEA (1.44 g, 14.27 mmol, 1.99 mL) in DMF (10 mL) was added HATU (1.74 g, 4.57 mmol, 2.39 mL). The resulting reaction mixture was stirred at 20° C. for 4 hr. Then, it was diluted with water (40 ml) and extracted with ethyl acetate (70 ml). Organic layer was washed successively with water (2×30 ml) and brine (30 ml), dried over Na₂SO₄ and concentrated under reduced pressure, affording methyl 2-oxo-2-[(2R,5S)-5-methyl-2-[2-(2-methyl-2-azaspiro[3.3]heptan-6-yl)-1,3-benzothiazol-5-yl]-1-piperidyl]acetate (1.24 g, crude).

LCMS(ESI): [M]⁺ m/z: calcd 427.2; found 428.2; Rt=1.051 min.

### Step 2: Synthesis of 2-((2R,5S)-5-methyl-2-(2-(2-methyl-2-azaspiro[3.3]heptan-6-yl)benzo[d]thiazol-5-yl)piperidin-1-yl)-2-oxoacetamide

Methyl 2-oxo-2-[(2R,5S)-5-methyl-2-[2-(2-methyl-2-azaspiro[3.3]heptan-6-yl)-1,3-benzothiazol-5-yl]-1-piperidyl]acetate (1.24 g, 2.90 mmol) was dissolved in ammonia (7N in MeOH) (15.58 g, 139.97 mmol, 20 mL, 15.3% purity). Resulting solution was stirred at 45° C. for 16 hr. Then, solvent was removed under reduced pressure, leaving 2-oxo-2-[(2R,5S)-5-methyl-2-[2-(2-methyl-2-azaspiro[3.3]heptan-6-yl)-1,3-benzothiazol-5-yl]-1-piperidyl]acetamide (1.21 g, crude). LCMS(ESI): [M]⁺ m/z: calcd 412.2; found 413.2; Rt=0.862 min.

### Intermediate 27. 2-oxo-2-[(2R,5S)-2-(3-chlorophenyl)-5-methyl-1-piperidyl]acetamide

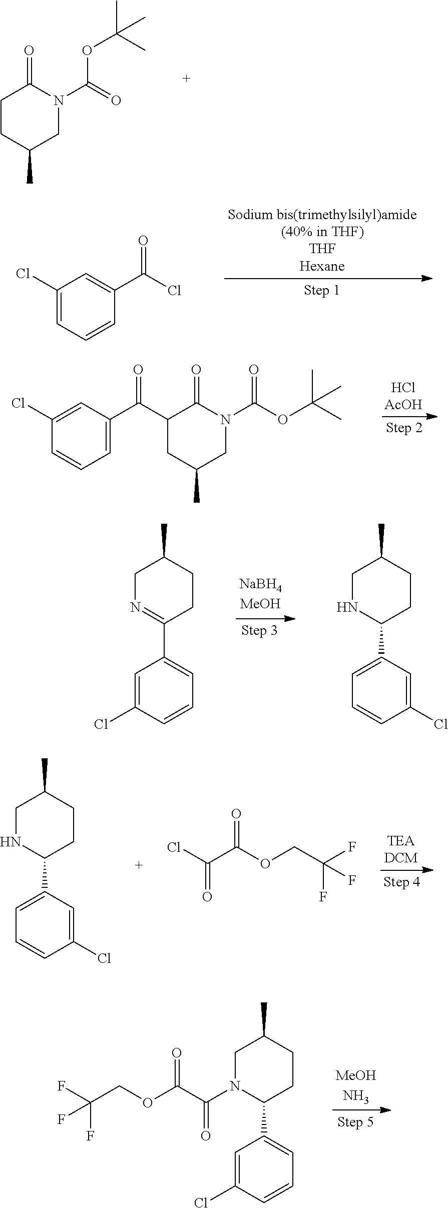

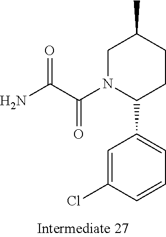

### Step 1: The synthesis of tert-butyl (5S)-3-(3-chlorobenzoyl)-5-methyl-2-oxo-piperidine-1-carboxylate

Sodium bis(trimethylsilyl)amide (40% in THF) (18.1 g, 39.4 mmol, 20.2 mL, 40% purity) was added dropwise to a solution of tert-butyl (5S)-5-methyl-2-oxo-piperidine-1-carboxylate (4.00 g, 18.8 mmol) in Tetrahydrofuran (35.0 mL) at −78° C. The reaction mixture was stirred at −78° C. for 1 hr. A solution of 3-chlorobenzoyl chloride (3.28 g, 18.8 mmol) in Hexane (10.0 mL) was added dropwise. The obtained mixture was slowly warmed up to 20° C. and stirred at this temperature for 2 hr. The reaction mixture was quenched with 15% aq. NaHSO₄ and extracted with ethyl acetate. The organic layer was washed with 20% aq. NaCl, dried over Na₂SO₄ and concentrated under reduced pressure to afford tert-butyl (5S)-3-(3-chlorobenzoyl)-5-methyl-2-oxo-piperidine-1-carboxylate (7.10 g, crude) as red oil.

LCMS(ESI): [M−H]⁺ m/z: calcd 350.15; found 250.0; Rt=1.369.

### Step 2: The synthesis of (3S)-6-(3-chlorophenyl)-3-methyl-2,3,4,5-tetrahydropyridine

Tert-butyl (5S)-3-(3-chlorobenzoyl)-5-methyl-2-oxo-piperidine-1-carboxylate (7.10 g, 20.2 mmol) was dissolved in Acetic acid (40.0 mL) and Hydrochloric acid, 36% w/w aq. soln. (28.6 g, 283 mmol, 35.8 mL, 36% purity) was added portion-wise. The obtained mixture was stirred at 100° C. for 12 hr. The reaction mixture was concentrated under reduced pressure. The residue was partitioned between IN HCl and MTBE. The organic layer was separated and discarded. The aqueous layer was basified to pH=10 with 10% NaOH and extracted with DCM. DCM layers were combined, dried over Na₂SO₄ and concentrated under reduced pressure to afford (3S)-6-(3-chlorophenyl)-3-methyl-2,3,4,5-tetrahydropyridine (2.23 g, 10.7 mmol, 53.2% yield) was obtained as a red liquid. LCMS(ESI): [M+H]⁺ m/z: calcd 208.11; found 208.2; Rt=0.614.

### Step 3: The synthesis of (2R,5S)-2-(3-chlorophenyl)-5-methyl-piperidine

To a solution of (3S)-6-(3-chlorophenyl)-3-methyl-2,3,4,5-tetrahydropyridine (2.23 g, 10.7 mmol) in MeOH (30.0 mL) Sodium Borohydride (406 mg, 10.7 mmol, 378 μL) was added portion-wise at 0° C. The resulting mixture was stirred for 2 hr at RT. The reaction mixture was concentrated in vacuo. The residue was diluted with water and extracted with DCM. The organic layer was dried over Na₂SO₄ and concentrated in vacuo to afford (2R,5S)-2-(3-chlorophenyl)-5-methyl-piperidine (1.90 g, 9.06 mmol, 84.4% yield) as red liquid.

LCMS(ESI): [M+H]⁺ m/z: calcd 210.13; found 210.0; Rt=0.854.

### Step 4: The synthesis of 2,2,2-trifluoroethyl 2-oxo-2-[rac-(2R,5S)-2-(3-chlorophenyl)-5-methyl-1-piperidyl]acetate

2,2,2-trifluoroethyl 2-chloro-2-oxo-acetate (363 mg, 1.91 mmol) was added dropwise to a solution of rac-(2R,5S)-2-(3-chlorophenyl)-5-methyl-piperidine (0.40 g, 1.91 mmol) and TEA (579 mg, 5.72 mmol, 798 μL) in DCM (20.0 mL) at 0° C. The resulting mixture was allowed to warm up to 20° C. and stirred for 12 hr. The reaction mixture was washed with water, dried over Na₂SO₄ and concentrated under reduced pressure to afford 2,2,2-trifluoroethyl 2-oxo-2-[rac-(2R,5S)-2-(3-chlorophenyl)-5-methyl-1-piperidyl]acetate (0.61 g, 1.68 mmol, 87.9% yield) as red gum. LCMS(ESI): [M+H]⁺ m/z: calcd 364.11; found 364.0; Rt=1.394.

### Step 5: The synthesis of 2-oxo-2-[(2R,5S)-2-(3-chlorophenyl)-5-methyl-1-piperidyl]acetamide

2,2,2-trifluoroethyl 2-oxo-2-[(2R,5S)-2-(3-chlorophenyl)-5-methyl-1-piperidyl]acetate (0.61 g, 1.68 mmol) was dissolved in MeOH(NH₃) (21.0 mL). The reaction mixture was stirred overnight. The obtained mixture was concentrated in vacuo to afford 2-((2R,5S)-2-(3-chlorophenyl)-5-methylpiperidin-1-yl)-2-oxoacetamide (0.42 g, 1.48 mmol, 88.2% yield) as light-yellow gum. LCMS(ESI): [M+H]⁺ m/z: calcd 281.12; found 281.0; Rt=1.101.

### Intermediate 28. 2-((2R,5S)-5-methyl-2-(3-(4-methylpiperazin-1-yl)phenyl)piperidin-1-yl)-2-oxoacetamide

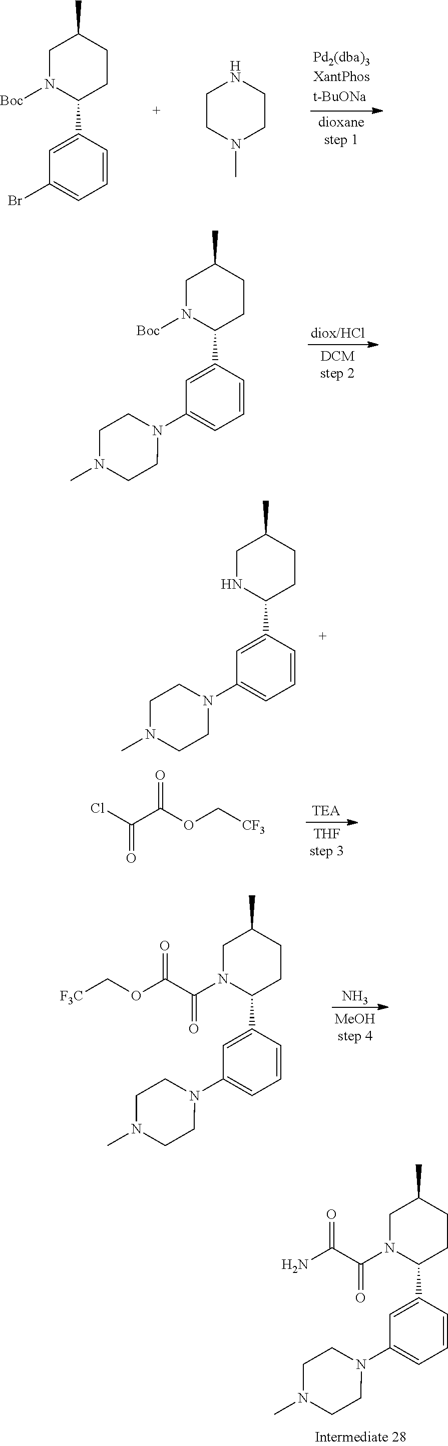

### Step 1: Synthesis of (2R,5S)-tert-butyl 5-methyl-2-(3-(4-methylpiperazin-1-yl)phenyl)piperidine-1-carboxylate

tert-Butyl (2R,5S)-2-(3-bromophenyl)-5-methyl-piperidine-1-carboxylate (1.03 g, 2.91 mmol), 1-methylpiperazine (291.20 mg, 2.91 mmol, 322.48 μL), 4,5-bis(diphenylphosphino)-9,9-dimethylxanthene (168.22 mg, 290.73 μmol) and sodium tert-butoxide (419.09 mg, 4.36 mmol) were mixed together in dioxane (15 mL) and the resulting mixture was evacuated and backfilled three times with argon. tris(Dibenzylideneacetone)dipalladium(0) (133.11 mg, 145.36 μmol) was added to the previous mixture and the resulting mixture was heated at 100° C. (oil bath) overnight. The reaction mixture was cooled and diluted with water (50 ml). The resulting mixture was extracted with EtOAc (3*50 ml). Combined organic layers were washed with brine (2*50 ml), dried over Na₂SO₄, filtered and evaporated to obtain tert-butyl (2R,5S)-5-methyl-2-[3-(4-methylpiperazin-1-yl)phenyl]piperidine-1-carboxylate (1.1 g, crude).

LCMS(ESI): [M]⁺ m/z: calcd 373.2; found 374.2; Rt=0.991 min.

### Step 2: Synthesis of 1-methyl-4-(3-((2R,5S)-5-methylpiperidin-2-yl)phenyl) piperazine

tert-Butyl (2R,5S)-5-methyl-2-[3-(4-methylpiperazin-1-yl)phenyl]piperidine-1-carboxylate (1.1 g, 2.94 mmol) was dissolved in DCM (15 mL), then dioxane/HCl (2.94 mmol, 5 mL) was added and it was stirred 16 hr at rt. Insoluble material was collected by filtration, filter cake was washed with additional amount of MTBE and dried on air to afford 1-methyl-4-[3-[(2R,5S)-5-methyl-2-piperidyl]phenyl]piperazine (750 mg, 2.42 mmol, 82.19% yield, HCl).

LCMS(ESI): [M]⁺ m/z: calcd 273.2; found 274.2; Rt=0.675 min.

### Step 3: Synthesis of 2,2,2-trifluoroethyl 2-((2R,5S)-5-methyl-2-(3-(4-methylpiperazin-1-yl)phenyl)piperidin-1-yl)-2-oxoacetate

To a solution of 1-methyl-4-[3-[(2R,5S)-5-methyl-2-piperidyl]phenyl]piperazine (750 mg, 2.42 mmol, HCl) and TEA (538.81 mg, 5.32 mmol, 742.16 μL) in THF (15.00 mL) was added 2,2,2-trifluoroethyl 2-chloro-2-oxo-acetate (461.08 mg, 2.42 mmol) at 0° C. After stirring at 20° C. for 16 hr the resulting mixture were evaporated to dryness and dissolved in DCM. Then, it was washed with water, dried over Na₂SO₄ and concentrated under reduced pressure, to give 2,2,2-trifluoroethyl 2-[(2R,5S)-5-methyl-2-[3-(4-methylpiperazin-1-yl)phenyl]-1-piperidyl]-2-oxo-acetate (715 mg, 1.67 mmol, 69.11% yield) as a brown solid, which was used in the next step without further purification.

LCMS(ESI): [M]⁺ m/z: calcd 427.2; found 428.2; Rt=0.980 min.

### Step 4: Synthesis of 2-((2R,5S)-5-methyl-2-(3-(4-methylpiperazin-1-yl)phenyl)piperidin-1-yl)-2-oxoacetamide

A solution of 2,2,2-trifluoroethyl 2-oxo-2-[(2R,5S)-5-methyl-2-[3-(4-methylpiperazin-1-yl)phenyl]-1-piperidyl]acetate (715 mg, 1.67 mmol) in MeOH/NH₃ (5N) (20 mL) was stirred at 20° C. for 18 hr. The solvent was evaporated to obtain 2-oxo-2-[(2R,5S)-5-methyl-2-[3-(4-methylpiperazin-1-yl)phenyl]-1-piperidyl]acetamide (528 mg, 1.53 mmol, 91.64% yield).

LCMS(ESI): [M]⁺ m/z: calcd 344.2; found 345.2; Rt=0.793 min.

### Intermediate 29. 2-[(2R,5S)-5-methyl-2-(2-methylindazol-6-yl)-1-piperidyl]-2-oxo-acetamide

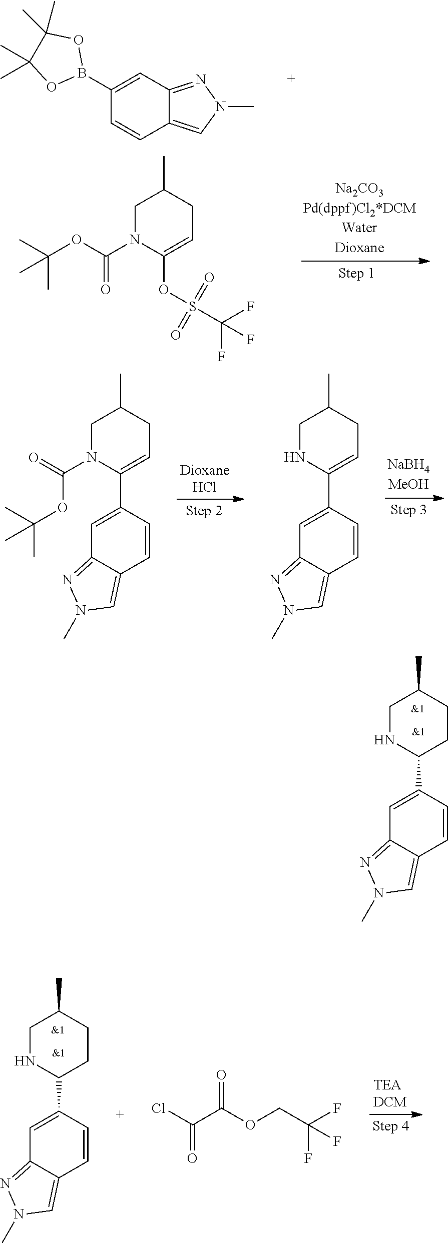

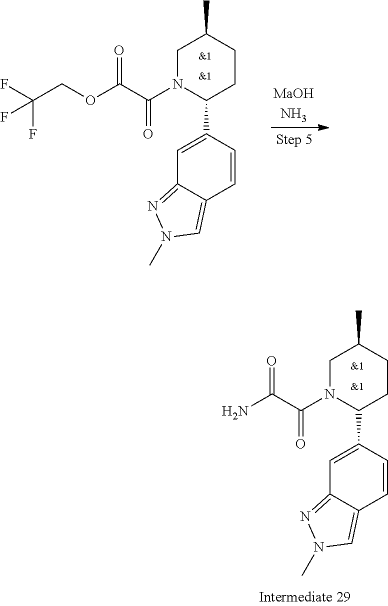

### Step 1: The synthesis of tert-butyl 3-methyl-6-(2-methylindazol-6-yl)-3,4-dihydro-2H-pyridine-1-carboxylate

2-methyl-6-(4,4,5,5-tetramethyl-1,3,2-dioxaborolan-2-yl)indazole (6.01 g, 23.3 mmol), tert-butyl 3-methyl-6-(trifluoromethylsulfonyloxy)-3,4-dihydro-2H-pyridine-1-carboxylate (8.85 g, 25.6 mmol) and Sodium carbonate (4.94 g, 46.6 mmol) were mixed in a mixture of Water (30.0 mL) and Dioxane (120 mL). The resulting mixture was evacuated and then backfilled with argon.

Cyclopentyl(diphenyl)phosphane; dichloromethane; dichloropalladium; iron (951 mg, 1.16 mmol) was added thereto. The reaction mixture was stirred at 90° C. for 16 hr. The obtained mixture was added to a solution of EtOAc (200 mL). The organic phase was washed with brine (3*100 mL) and concentrated under reduced pressure. The residue was diluted with MTBE (200 mL). The obtained mixture was stirred for 30 minutes and filtered off. The filtrate was concentrated in vacuo to afford tert-butyl 3-methyl-6-(2-methylindazol-6-yl)-3,4-dihydro-2H-pyridine-1-carboxylate (7.80 g, crude) as a red solid. LCMS(ESI): [M+H]⁺ m/z: calcd 328.23; found 328.4; Rt=1.357.

### Step 2: The synthesis of 2-methyl-6-(3-methyl-1,2,3,4-tetrahydropyridin-6-yl)indazole

A mixture of tert-butyl 3-methyl-6-(2-methylindazol-6-yl)-3,4-dihydro-2H-pyridine-1-carboxylate (7.80 g, 23.8 mmol) and Dioxane/HCl (100 mL) was stirred at room temperature for 24° C. The reaction mixture was concentrated in vacuo. The residue was poured into water (20.0 mL) and basified with K₂CO₃ to pH˜10. The aqueous phase was extracted with CHCl₃ (3*15.0 mL). The combined organic layers were dried and concentrated in vacuo to afford 2-methyl-6-(3-methyl-1,2,3,4-tetrahydropyridin-6-yl)indazole (5.00 g, crude). LCMS(ESI): [M+H]⁺ m/z: calcd 228.17; found 228.2; Rt=0.678.

### Step 3: The synthesis of 2-methyl-6-[(2R,5S)-5-methyl-2-piperidyl]indazole

To a mixture of 2-methyl-6-(3-methyl-1,2,3,4-tetrahydropyridin-6-yl)indazole (5.00 g, 22.0 mmol) in MeOH (100 mL) Sodium Borohydride (1.25 g, 33.0 mmol) was added at 0° C. The resulting mixture was allowed to stir for 72 hr at r.t. The obtained mixture was concentrated in vacuo. The residue was quenched with water (80.0 mL). The aqueous phase was extracted with CHCl₃ (3*30.0 mL). The combined organic phase was dried over Na₂SO₄ and concentrated under reduced pressure to afford 2-methyl-6-[(2R,5S)-5-methyl-2-piperidyl]indazole (4.30 g, crude) as black oil. LCMS(ESI): [M+H]⁺ m/z: calcd 230.19; found 230.2; Rt=0.895.

### Step 4: The synthesis of 2,2,2-trifluoroethyl 2-[(2R,5S)-5-methyl-2-(2-methylindazol-6-yl)-1-piperidyl]-2-oxo-acetate

2-methyl-6-[(2R,5S)-5-methyl-2-piperidyl]indazole (2.00 g, 8.72 mmol) was added dropwise to a mixture of 2-methyl-6-[(2R,5S)-5-methyl-2-piperidyl]indazole (2.00 g, 8.72 mmol) and triethylamine (1.06 g, 10.5 mmol, 1.46 mL) in DCM (50.0 mL) at 0° C. The resulting mixture was stirred for 1 hour with TLC control. The organic phase was washed with brine (2*30.0 mL), separated, dried and concentrated in vacuo to afford 2,2,2-trifluoroethyl 2-[(2R,5S)-5-methyl-2-(2-methylindazol-6-yl)-1-piperidyl]-2-oxo-acetate (2.95 g, 7.70 mmol, 88.2% yield) as black oil. LCMS(ESI): [M+H]⁺ m/z: calcd 384.17; found 384.2; Rt=1.231.

### Step 5: The synthesis of 2-[(2R,5S)-5-methyl-2-(2-methylindazol-6-yl)-1-piperidyl]-2-oxo-acetamide

A mixture of 2,2,2-trifluoroethyl 2-[(2R,5S)-5-methyl-2-(2-methylindazol-6-yl)-1-piperidyl]-2-oxo-acetate (0.90 g, 2.35 mmol) in MeOH/NH₃ (15.0 mL) was allowed to stir overnight at 25° C. The obtained mixture was concentrated under reduced pressure to afford 2-[(2R,5S)-5-methyl-2-(2-methylindazol-6-yl)-1-piperidyl]-2-oxo-acetamide (0.40 g, crude) as a brown solid. LCMS(ESI): [M+H]⁺ m/z: calcd 301.18; found 301.2; Rt=1.023.

### Intermediate 30. rac-(2R,5R)-2-(4-Fluorophenyl)-4-methoxy-5-methyl-piperidine

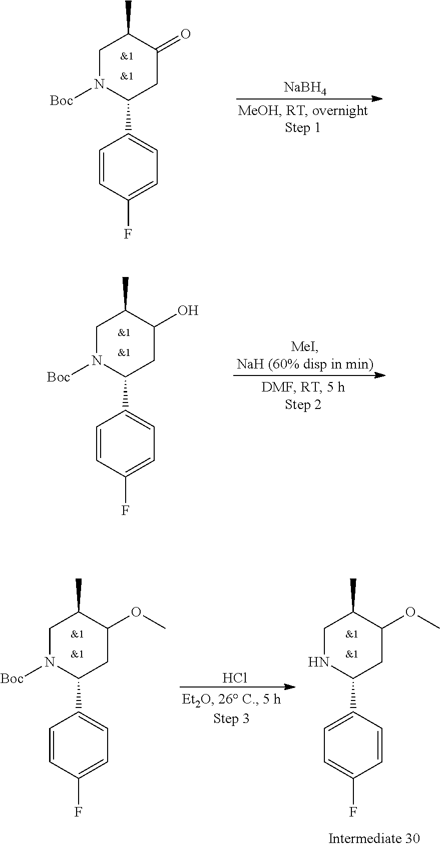

### Step 1: Synthesis of rac-tert-butyl (2R,5R)-2-(4-fluorophenyl)-4-hydroxy-5-methyl-piperidine-1-carboxylate

Sodium Borohydride (221.56 mg, 5.86 mmol, 207.07 μL) was added portionwise to tert-butyl (2R,5R)-2-(4-fluorophenyl)-5-methyl-4-oxo-piperidine-1-carboxylate (3.60 g, 11.71 mmol) in dry MeOH (36 mL) and then warmed to room temperature overnight. The reaction was quenched by the addition of a saturated aqueous solution of ammonium chloride. Extract with ethyl acetate (15 mL×3) and combine organic layers. Wash with saturated brine and dry over anhydrous sodium sulfate. Filtration and concentration of the filtrate under reduced pressure afforded tert-butyl (2R,5R)-2-(4-fluorophenyl)-4-hydroxy-5-methyl-piperidine-1-carboxylate (3.52 g, 11.38 mmol, 97.14% yield) tert-butyl (2R,5R)-2-(4-fluorophenyl)-4-hydroxy-5-methyl-piperidine-1-carboxylate (3.52 g, 11.38 mmol, 97.14% yield). LCMS(ESI): [M-Boc]⁺ m/z: calcd 210.2; found 210.2; Rt=1.240 min.

### Step 2: The Synthesis of rac-tert-Butyl (2R,5R)-2-(4-fluorophenyl)-4-methoxy-5-methyl-piperidine-1-carboxylate

Sodium hydride (in oil dispersion) 60% dispersion in mineral oil (73.35 mg, 3.19 mmol) was added to a solution of tert-butyl (2R,5R)-2-(4-fluorophenyl)-4-hydroxy-5-methyl-piperidine-1-carboxylate (329 mg, 1.06 mmol) in DMF (5 mL). The mixture was stirred for 30 minutes following by the addition of iodomethane (452.83 mg, 3.19 mmol, 198.61 μL). The mixture was stirred for 5 h. Water (10 mL) was added and the mixture was extracted with diethyl ether (2×10 mL). The combined organic fractions were washed with water (4×10 mL) and brine, dried (MgSO₄) and the solvent was evaporated under reduced pressure to give the title compound tert-butyl (2R,5R)-2-(4-fluorophenyl)-4-methoxy-5-methyl-piperidine-1-carboxylate (303 mg, 936.92 μmol, 88.10% yield). LCMS(ESI): [M-Boc]⁺ m/z: calcd 224.2; found 224.2; Rt=1.525 min.

### Step 3: The Synthesis of rac-(2R,5R)-2-(4-Fluorophenyl)-4-methoxy-5-methyl-piperidine

To tert-butyl (2R,5R)-2-(4-fluorophenyl)-4-methoxy-5-methyl-piperidine-1-carboxylate (303.00 mg, 936.92 μmol) was added ET₂O/HCl (10 mL) and the mixture was stirred at 26° C. for 5 hr. After 5 hours solvent was evaporated to obtain (2R,5R)-2-(4-fluorophenyl)-4-methoxy-5-methyl-piperidine (223 mg, crude). LCMS(ESI): [M+H]⁺ m/z: calcd 224.2; found 224.2; Rt=0.666 min.

### Intermediate 31. 2-(1,3-dimethyl-4-piperidyl)-5-[(5S)-5-methyl-2-piperidyl]-1,3-benzothiazole

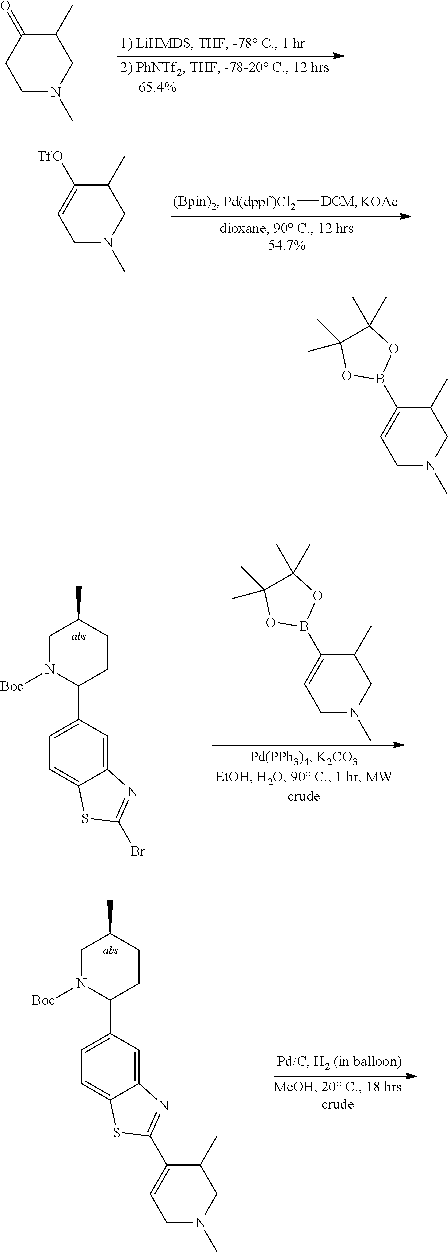

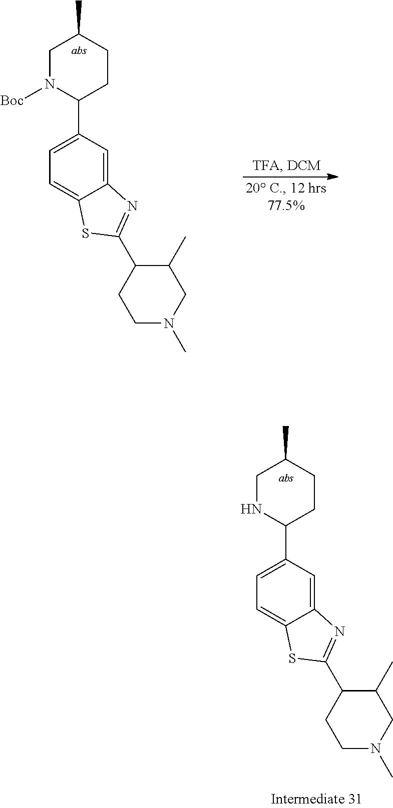 [SMILES: CC1CN(C)CCC1c1nc2cc(C3CC[C@H](C)CN3C(=O)OC(C)(C)C)ccc2s1 ; CC1CN(C)CCC1c1nc2cc(C3CC[C@H](C)CN3)ccc2s1]

### Step 1: Synthesis of (1,3-dimethyl-3,6-dihydro-2H-pyridin-4-yl) trifluoromethanesulfonate

To a mixture of 1,3-dimethylpiperidin-4-one (1.5 g, 0.0118 mol) in THF (10 mL) was sealed and degassed under vacuum and purged with N₂ for three times, and then 1M lithium; bis(trimethylsilyl) azanide/THF (17 mL, 17.0 mmol) was added at −78° C. dropwise, the mixture was stirred for 1 hour at −78° C., then PhNTf₂ (6.3 g, 0.176 mol) in THF (10 mL) was added. The solution was stirred at 20° C. for 12 hours. The resulting mixture was quenched by addition of water (50 mL) and extracted with EtOAc (50 mL*3). The combined organic layer was washed with saturated NH₄Cl aqueous solution (50 mL*2), brine (50 mL), dried over anhydrous Na₂SO₄, filtered and concentrated under reduced pressure. The residue was purified by flash chromatography (ISCO®; 24 g AgelaFlash® Silica Flash Column, petroleum ether/EtOAc with EtOAc from 0˜80%, flow rate=30 mL/min, 254 nm) to afford (1,3-dimethyl-3,6-dihydro-2H-pyridin-4-yl) trifluoromethanesulfonate (2 g, 65.4% yield) as yellow oil. ¹H NMR (400 MHZ, methanol-d₄) δ ppm 5.83 (br s, 1H), 3.21-3.28 (m, 1H), 3.06-3.21 (m, 1H), 2.99 (br dd, J=11.5, 5.3 Hz, 1H), 2.80 (br s, 1H), 2.36-2.48 (m, 4H), 1.16 (d, J=7.0 Hz, 3H). LCMS(ESI) [M+H]⁺ m/z: calcd 260.0, found 260.0.

### Step 2: Synthesis of 1,3-dimethyl-4-(4,4,5,5-tetramethyl-1,3,2-dioxaborolan-2-yl)-3,6-dihydro-2H-pyridine

To a solution of (1,3-dimethyl-3,6-dihydro-2H-pyridin-4-yl) trifluoromethanesulfonate (2 g, 7.71 mmol), 4,4,5,5-tetramethyl-2-(4,4,5,5-tetramethyl-1,3,2-dioxaborolan-2-yl)-1,3,2-dioxaborolane (2.43 g, 9.56 mmol), cyclopentyl(diphenyl)phosphane; dichloromethane; dichloropalladium; iron (650 mg, 0.796 mmol) in dioxane (20 mL) was added CH₃COOK (2.29 g, 0.0233 mol). The resulting mixture was sealed and degassed under vacuum and purged with N₂ for three times, and then stirred at 90° C. for 12 hours under N₂. The resulting mixture was quenched by addition of water (50 mL) and extracted with EtOAc (50 mL*3). The combined organic layer was washed with saturated NH₄Cl aqueous solution (50 mL*2), brine (50 mL), dried over anhydrous Na₂SO₄, filtered and concentrated under reduced pressure. The residue was purified by flash chromatography (ISCO®; 24 g AgelaFlash® Silica Flash Column, EtOAc/MeOH with MeOH from 0˜20%, flow rate=30 mL/min, 254 nm) to afford 1,3-dimethyl-4-(4,4,5,5-tetramethyl-1,3,2-dioxaborolan-2-yl)-3,6-dihydro-2H-pyridine (1 g, 54.7% yield) as brown solid. LCMS (ESI) [M+H]⁺ m/z: calcd 238.2, found 238.1.

### Step 3: Synthesis of tert-butyl (5S)-2-[2-(1,3-dimethyl-3,6-dihydro-2H-pyridin-4-yl)-1,3-benzothiazol-5-yl]-5-methyl-piperidine-1-carboxylate

tert-butyl (5S)-2-(2-bromo-1,3-benzothiazol-5-yl)-5-methyl-piperidine-1-carboxylate (300 mg, 0.729 mmol), 1,3-dimethyl-4-(4,4,5,5-tetramethyl-1,3,2-dioxaborolan-2-yl)-3,6-dihydro-2H-pyridine (300 mg, 1.27 mmol), K₂CO₃ (300 mg, 2.17 mmol), and Pd(PPh₃)₄ (120 mg, 0.104 mmol) were taken up into a microwave tube in EtOH (10 mL) and H₂O (1 mL). The sealed tube was heated at 90° C. for 1 hour in a microwave. The resulting mixture was quenched by addition of water (20 mL) and extracted with EtOAc (20 mL*3). The combined organic layer was washed with saturated NH₄Cl aqueous solution (20 mL*2), brine (20 mL), dried over anhydrous Na₂SO₄, filtered and concentrated under reduced pressure. The residue was purified by flash chromatography (ISCO®; 24 g AgelaFlash® Silica Flash Column, petroleum ether/EtOAc with EtOAc from 0˜100%, flow rate=30 mL/min, 254 nm) to afford tert-butyl (5S)-2-[2-(1,3-dimethyl-3,6-dihydro-2H-pyridin-4-yl)-1,3-benzothiazol-5-yl]-5-methyl-piperidine-1-carboxylate (300 mg, crude) as brown oil. LCMS (ESI) [M+H]⁺ m/z: calcd 442.2, found 442.2.

### Step 4: Synthesis of tert-butyl (5S)-2-[2-(1,3-dimethyl-4-piperidyl)-1,3-benzothiazol-5-yl]-5-methyl-piperidine-1-carboxylate

To a solution of tert-butyl (5S)-2-[2-(1,3-dimethyl-3,6-dihydro-2H-pyridin-4-yl)-1,3-benzothiazol-5-yl]-5-methyl-piperidine-1-carboxylate (300 mg, 0.679 mmol) in MeOH (4 mL) was added Pd/C (300 mg, 10% Pd/C with 50% of water, wt %). The resulting mixture was sealed and degassed under vacuum and purged with H₂ for three times, and then stirred at 20° C. for 18 hours under H₂ (in balloon). The resulting mixture was filtered and concentrated under reduced pressure to give tert-butyl (5S)-2-[2-(1,3-dimethyl-4-piperidyl)-1,3-benzothiazol-5-yl]-5-methyl-piperidine-1-carboxylate (250 mg, crude) as yellow oil.

LCMS(ESI) [M+H]⁺ m/z: calcd 442.2, found 444.3.

### Step 5: Synthesis of 2-(1,3-dimethyl-4-piperidyl)-5-[(5S)-5-methyl-2-piperidyl]-1,3-benzothiazole

To a solution of tert-butyl (5S)-2-[2-(1,3-dimethyl-4-piperidyl)-1,3-benzothiazol-5-yl]-5-methyl-piperidine-1-carboxylate (250 mg, 0.564 mmol) in DCM (6 mL) was added TFA (0.8 mL, 10.4 mmol). The mixture was stirred at 20° C. for 12 hours. The resulting mixture concentrated under reduced pressure to afford 2-(1,3-dimethyl-4-piperidyl)-5-[(5S)-5-methyl-2-piperidyl]-1,3-benzothiazole (150 mg, 77.5% yield) as yellow oil.

### Intermediate 32. 2-(1-methyl-4-piperidyl)-5-[(5S)-5-methyl-2-piperidyl]-1,3-benzothiazole

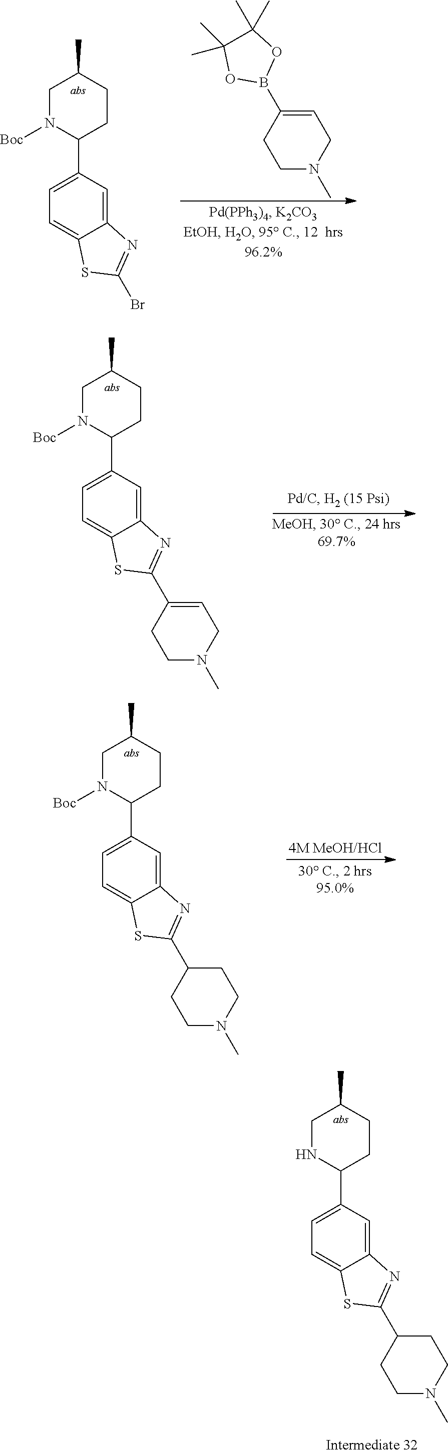

### Step 1: Synthesis of tert-butyl (5S)-5-methyl-2-[2-(1-methyl-3,6-dihydro-2H-pyridin-4-yl)-1,3-benzothiazol-5-yl]piperidine-1-carboxylate

A mixture of tert-butyl (5S)-2-(2-bromo-1,3-benzothiazol-5-yl)-5-methyl-piperidine-1-carboxylate (10 g, 24.3 mmol), 1-methyl-4-(4,4,5,5-tetramethyl-1,3,2-dioxaborolan-2-yl)-3,6-dihydro-2H-pyridine (8 g, 35.9 mmol), K₂CO₃ (10 g, 72.4 mmol), palladium; triphenylphosphane (3 g, 2.60 mmol), EtOH (100 mL) and H₂O (20 mL) was stirred at 95° C. for 12 hours. The resulting mixture was quenched by addition of water (50 mL) and extracted with EtOAc (100 mL*3). The combined organic layer was washed with saturated NH₄Cl aqueous solution (50 mL*2), brine (50 mL), dried over anhydrous Na₂SO₄, filtered and concentrated under reduced pressure. The residue was purified by flash chromatography (ISCOR; 80 g AgelaFlash® Silica Flash Column, DCM/MeOH with MeOH from 0˜20%, Flow Rate: 30 mL/min) to afford tert-butyl (5S)-5-methyl-2-[2-(1-methyl-3,6-dihydro-2H-pyridin-4-yl)-1,3-benzothiazol-5-yl]piperidine-1-carboxylate (10 g, 96.2% yield) as yellow solid. ¹H NMR (400 MHZ, methanol-d₄) δ ppm 7.89-7.97 (m, 1H), 7.78-7.84 (m, 1H), 7.29-7.40 (m, 1H), 6.80 (s, 1H), 5.39 (s, 1H), 3.78 (d, J=13.6 Hz, 1H), 3.14 (dd, J=13.6, 3.8 Hz, 1H), 2.73-2.91 (m, 5H), 2.41-2.57 (m, 6H), 1.74-1.96 (m, 4H), 1.47 (s, 8H), 1.06-1.11 (m, 3H).

### Step 2: Synthesis of tert-butyl (5S)-5-methyl-2-[2-(1-methyl-4-piperidyl)-1,3-benzothiazol-5-yl]piperidine-1-carboxylate

A mixture of tert-butyl (5S)-5-methyl-2-[2-(1-methyl-3,6-dihydro-2H-pyridin-4-yl)-1,3-benzothiazol-5-yl]piperidine-1-carboxylate (10 g, 23.4 mmol), Pd/C (10 g, 10% of Pd with 50% of water, wt %) and MeOH (100 mL) was stirred at 30° C. for 24 hours (15 Psi). The mixture was filtered and concentrated under reduced pressure to give tert-butyl (5S)-5-methyl-2-[2-(1-methyl-4-piperidyl)-1,3-benzothiazol-5-yl]piperidine-1-carboxylate (7 g, 69.8% yield) as yellow oil. ¹H NMR (400 MHZ, methanol-d₄) δ ppm 7.92-7.99 (m, 1H), 7.77-7.86 (m, 1H), 7.28-7.40 (m, 1H), 5.40 (br t, J=4.5 Hz, 1H), 3.78 (br d, J=13.6 Hz, 1H), 3.09-3.19 (m, 2H), 3.02 (br d, J=11.8 Hz, 2H), 2.74-2.87 (m, 1H), 2.17-2.28 (m, 5H), 1.75-2.02 (m, 5H), 1.43-1.51 (m, 10H), 1.32-1.40 (m, 2H), 1.09 (d, J=6.8 Hz, 3H).

### Step 3: Synthesis of 2-(1-methyl-4-piperidyl)-5-[(5S)-5-methyl-2-piperidyl]-1,3-benzothiazole

A mixture of tert-butyl (5S)-5-methyl-2-[2-(1-methyl-4-piperidyl)-1,3-benzothiazol-5-yl]piperidine-1-carboxylate (7 g, 16.3 mmol) and 4M HCl/MeOH (19.9 mL, 79.6 mmol) was stirred at 30° C. for 2 hours. The mixture was concentrated under reduced pressure to give 2-(1-methyl-4-piperidyl)-5-[(5S)-5-methyl-2-piperidyl]-1,3-benzothiazole (5.1 g, 95.0% yield) as yellow solid. LCMS(ESI) [M+H]⁺ m/z: calcd 330.2, found 330.2.

### Intermediate 33. 5-((2R,5S)-5-methylpiperidin-2-yl)-2-(1,2,2-trimethylpiperidin-4-yl)benzo[d]thiazole

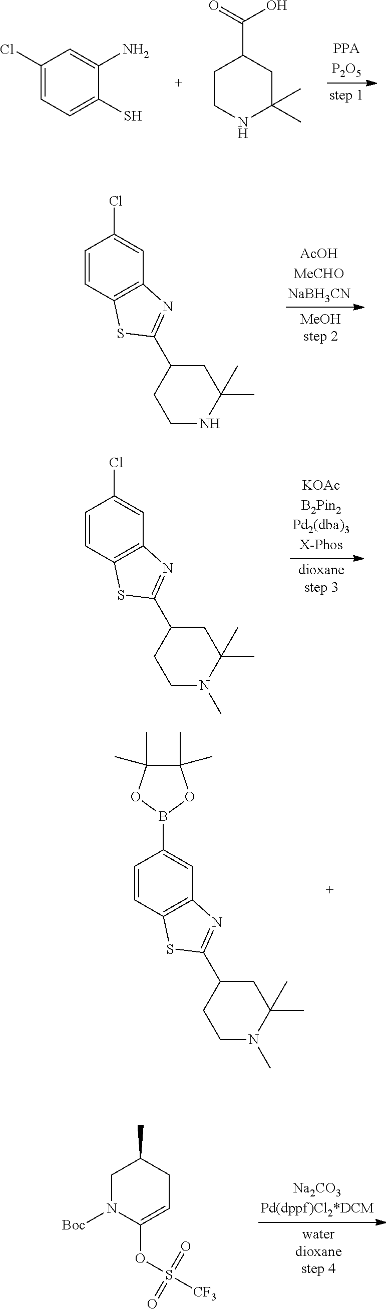

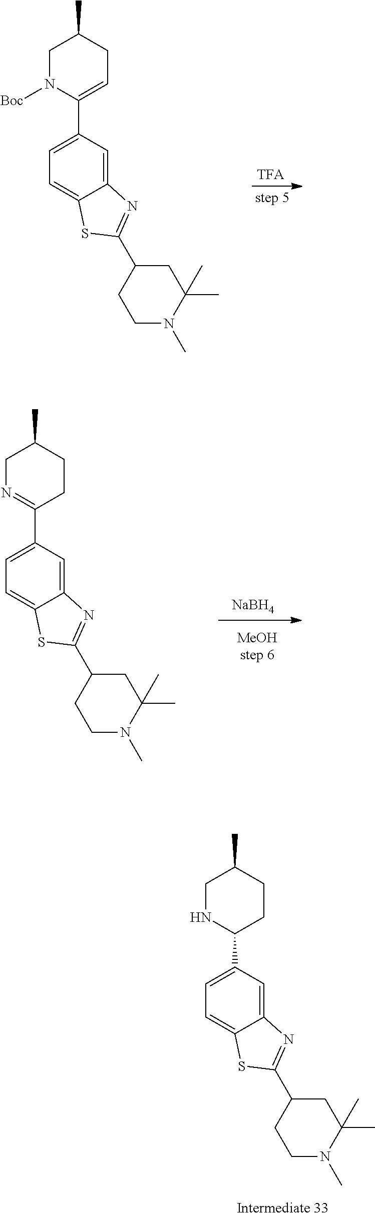 [SMILES: C[C@H]1CC=C(c2ccc3sc(C4CCN(C)C(C)(C)C4)nc3c2)N(C(=O)OC(C)(C)C)C1 ; C[C@H]1CCC(c2ccc3sc(C4CCN(C)C(C)(C)C4)nc3c2)=NC1 ; "NaBH4" ; C[C@H]1CC[C@H](c2ccc3sc(C4CCN(C)C(C)(C)C4)nc3c2)NC1]

### Step 1: Synthesis of 5-chloro-2-(2,2-dimethylpiperidin-4-yl)benzo[d]thiazole

Prepared by general procedure scheme S2 step 1A. Yield: 6.8 g (77.52%).

CC conditions: The crude product was purified by silica gel with MTBE/MeOH as an eluent mixture.

LCMS(ESI): [M]⁺ m/z: calcd 280.2; found 281.2; Rt=0.859 min.

### Step 2: Synthesis of 5-chloro-2-(1,2,2-trimethylpiperidin-4-yl)benzo[d]thiazole

Formaldehyde, 37% w/w aq. soln., stab. with 7-8% MeOH (577.47 mg, 19.23 mmol, 533.21 μL) and acetic acid (1.15 g, 19.23 mmol, 1.10 mL) were added to the solution of 5-chloro-2-(2,2-dimethyl-4-piperidyl)-1,3-benzothiazole (2.7 g, 9.61 mmol) in MeOH (60 mL). Resulting mixture was stirred at 20° C. for 1 hr before sodium cyan borohydride (1.21 g, 19.23 mmol) was added thereto. After that, stirring was continued for 16 hr. Then, solvent was removed under reduced pressure and residue was partitioned between 10% aq. K₂CO₃ solution (20 ml) and DCM (40 ml). Organic layer was separated, dried over solid K₂CO₃ and concentrated under reduced pressure, leaving 5-chloro-2-(1,2,2-trimethyl-4-piperidyl)-1,3-benzothiazole (2.7 g, 9.16 mmol, 95.24% yield). LCMS(ESI): [M]⁺ m/z: calcd 294.2; found 295.2; Rt=1.019 min.

### Step 3: Synthesis of 5-(4,4,5,5-tetramethyl-1,3,2-dioxaborolan-2-yl)-2-(1,2,2-trimethylpiperidin-4-yl)benzo[d]thiazole

5-Chloro-2-(1,2,2-trimethyl-4-piperidyl)-1,3-benzothiazole (2.7 g, 9.16 mmol), 4,4,5,5-tetramethyl-2-(4,4,5,5-tetramethyl-1,3,2-dioxaborolan-2-yl)-1,3,2-dioxaborolane (2.67 g, 10.53 mmol) and potassium acetate (1.80 g, 18.31 mmol, 1.14 mL) were mixed together in dioxane (50 mL). Reaction flask was evacuated and refilled with argon 3 times. Then, XPhos (873.10 mg, 1.83 mmol) and tris(dibenzylideneacetone)dipalladium(0) (419.28 mg, 457.87 μmol) were added under stream of argon. Resulting mixture was stirred at 100° C. for 16 hr. Then, it was concentrated under reduced pressure and residue was purified by gradient column chromatography (SiO₂, MTBE/MeOH), affording 5-(4,4,5,5-tetramethyl-1,3,2-dioxaborolan-2-yl)-2-(1,2,2-trimethyl-4-piperidyl)-1,3-benzothiazole (1.96 g, 5.07 mmol, 55.40% yield). LCMS(ESI): [M]⁺ m/z: calcd 386.2; found 387.2; Rt=1.101 min.

### Step 4: Synthesis of (3S)-tert-butyl 3-methyl-6-(2-(1,2,2-trimethylpiperidin-4-yl)benzo[d]thiazol-5-yl)-3,4-dihydropyridine-1(2H)-carboxylate

Prepared by general procedure scheme S2 step 3. Yield: 2.5 g of crude.

LCMS(ESI): [M]⁺ m/z: calcd 455.2; found 456.2; Rt=1.357 min.

### Step 5: Synthesis of 5-((S)-5-methyl-3,4,5,6-tetrahydropyridin-2-yl)-2-(1,2,2-trimethylpiperidin-4-yl)benzo[d]thiazole

Prepared by general procedure scheme S2 step 4. Yield: 1.6 g of crude.

LCMS(ESI): [M]⁺ m/z: calcd 355.2; found 356.2; Rt=0.716 min.

### Step 6: Synthesis of 5-((2R,5S)-5-methylpiperidin-2-yl)-2-(1,2,2-trimethylpiperidin-4-yl)benzo[d]thiazole

Prepared by general procedure scheme S2 step 5. Yield: 1.2 g (74.58%).

LCMS(ESI): [M]⁺ m/z: calcd 357.2; found 358.2; Rt=0.715 min.

### Intermediate 34.2-[(3-methylimidazo[1,2-a]pyridin-7-yl)amino]-2-oxo-acetic acid

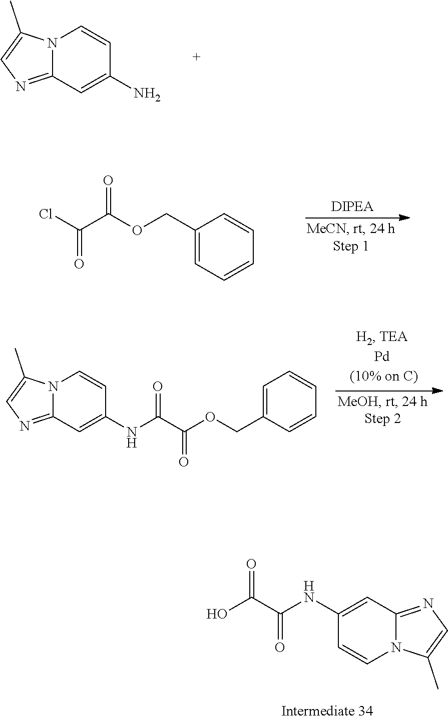 [SMILES: Cc1cnc2cc(N)ccn12 ; O=C(Cl)C(=O)OCc1ccccc1 ; Cc1cnc2cc(NC(=O)C(=O)OCc3ccccc3)ccn12 ; Cc1cnc2cc(NC(=O)C(=O)O)ccn12]

### Step 1. Synthesis of benzyl 2-[(3-methylimidazo[1,2-a]pyridin-7-yl)amino]-2-oxo-acetate

A solution of benzyl 2-chloro-2-oxo-acetate (1.30 g, 6.54 mmol) in MeCN was added dropwise at 0° C. to a stirred solution of 3-methylimidazo[1,2-a]pyridin-7-amine (1.2 g, 5.45 mmol, 2HCl) and DIPEA (3.52 g, 27.26 mmol, 4.75 mL) in MeCN (100 mL). The reaction mixture was allowed to warm to 25° C. and stirred for 12 hr. Then probe was evaporated in vacuo and starting amine was detected. benzyl 2-chloro-2-oxo-acetate (1.30 g, 6.54 mmol) and DIPEA (3.52 g, 27.26 mmol, 4.75 mL) were added. The reaction mixture was allowed to warm to 25° C. and stirred for 12 hr. Then reaction was evaporated in vacuo. The residue was washed with water and dried to afford benzyl 2-[(3-methylimidazo[1,2-a]pyridin-7-yl)amino]-2-oxo-acetate (1.4 g, 4.53 mmol, 83.02% yield) as yellow solid. LCMS(ESI): [M+1]⁺ m/z: calcd 310.2; found 310.2; Rt=0.907 min.

### Step 2. Synthesis of 2-[(3-methylimidazo[1,2-a]pyridin-7-yl)amino]-2-oxo-acetic acid

A mixture of benzyl 2-[(3-methylimidazo[1,2-a]pyridin-7-yl)amino]-2-oxo-acetate (0.4 g, 1.29 mmol) and Palladium on carbon, 10% (13.76 mg, 129.32 μmol) in MeOH (74.47 mL) was stirred under atmosphere of hydrogen at 25° C. for 24 hr. The resulting suspension of product and catalyst was filtered, washed with methanol (2*100 ml), and then again suspended in methanol (500 mL). TEA (392.57 mg, 3.88 mmol, 540.72 μL) was added and the resulting mixture was stirred for 0.1 hr. The resulting solution of triethyl ammonium salt of the product was filtered from catalyst and concentrated in vacuo to afford 2-[(3-methylimidazo[1,2-a]pyridin-7-yl)amino]-2-oxo-acetic acid (0.2 g, 912.42 μmol, 70.56% yield) as light-yellow solid. LCMS(ESI): [M+1]⁺ m/z: calcd 220.2; found 220.2; Rt=0.584 min.

### Intermediate 35. 2-oxo-2-[(5-oxo-6,7-dihydropyrrolo[3,4-b]pyridin-3-yl)amino]acetic acid

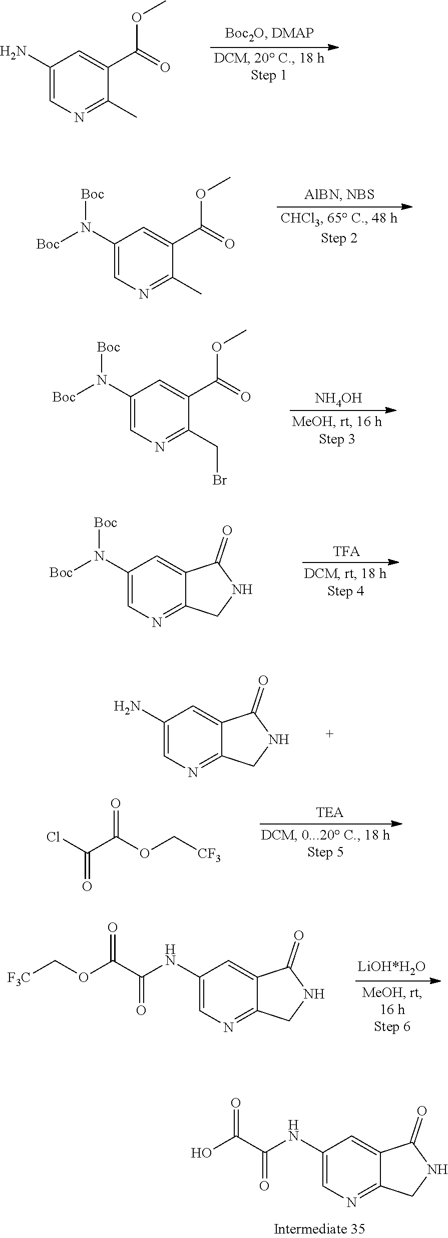

### Step 1. Synthesis of methyl 5-[bis(tert-butoxycarbonyl)amino]-2-methyl-pyridine-3-carboxylate

Di-tert-butyl dicarbonate (16.42 g, 75.22 mmol, 17.26 mL) was added dropwise to a solution of methyl 5-amino-2-methyl-pyridine-3-carboxylate (5 g, 30.09 mmol) and 4-Dimethylaminopyridine (183.80 mg, 1.50 mmol) in DCM (120.00 mL). Resulting solution was stirred for 18 hr at 20° C. and diluted with water. The organic layer was separated and washed with aq.citric acid solution (2*50 ml), dried over Na₂SO₄, filtered and evaporated. Resulting crude material was purified by column chromatography (Interchim 120 g SiO₂, HEX-MTBE from 0˜100%, flow rate=70 mL/min, cv=12.8) to obtain methyl 5-[bis(tert-butoxycarbonyl)amino]-2-methyl-pyridine-3-carboxylate (1.6 g, 4.37 mmol, 14.51% yield) and methyl 5-(tert-butoxycarbonylamino)-2-methyl-pyridine-3-carboxylate (5.4 g, 20.28 mmol, 67.40% yield). ¹H NMR (400 MHZ, DMSO-d₆) δ (ppm) 1.37 (s, 18H), 2.69 (s, 3H), 3.85 (s, 3H), 8.00 (s, 1H), 8.49 (s, 1H). LCMS(ESI): [M+1]⁺ m/z: calcd 367.2; found 367.2; Rt=1.383 min.

### Step 2. Synthesis of methyl 5-[bis(tert-butoxycarbonyl)amino]-2-(bromomethyl)pyridine-3-carboxylate

Methyl 5-[bis(tert-butoxycarbonyl)amino]-2-methyl-pyridine-3-carboxylate (1.39 g, 3.79 mmol), 2,2′-Azobis(2-methylpropionitrile) (124.59 mg, 758.72 μmol) and N-Bromosuccinimide (810.22 mg, 4.55 mmol, 386.19 μL) were dissolved in Chloroform (30 mL), and the mixture was heated at 65° C. for 48 hr. After ice-cooling the reaction mixture, the solvent was evaporated and resulting residue was diluted with MTBE. The precipitate was separated by filtration, and the filtrate was concentrated under reduced pressure to obtain methyl 5-[bis(tert-butoxycarbonyl)amino]-2-(bromomethyl)pyridine-3-carboxylate (1.5 g, crude) that was used without further purification. LCMS(ESI): [M+1]⁺ m/z: calcd 455.0; found 455.0; Rt=1.556 min.

### Step 3. Synthesis of tert-butyl N-tert-butoxycarbonyl-N-(5-oxo-6,7-dihydropyrrolo[3,4-b]pyridin-3-yl)carbamate

The mixture of methyl 5-[bis(tert-butoxycarbonyl)amino]-2-(bromomethyl)pyridine-3-carboxylate (1.5 g, 3.37 mmol), Ammonium hydroxide, 28% solution (3.54 g, 101.05 mmol, 3.94 mL) and Methanol (20 mL) was stirred at 25° C. for 16 hr. Resulting solution was diluted with water (20 ml) and resulting precipitate was collected by filtration to obtain tert-butyl N-tert-butoxycarbonyl-N-(5-oxo-6,7-dihydropyrrolo[3,4-b]pyridin-3-yl)carbamate (592 mg, crude). ¹H NMR (400 MHz, DMSO-d₆) δ (ppm) 1.47 (s, 18H), 4.46 (s, 2H), 8.01 (s, 1H), 8.61 (s, 1H), 8.88 (s, 1H). LCMS(ESI): [M+1]⁺ m/z: calcd 350.2; found 350.2; Rt=1.197 min.

### Step 4. Synthesis of 3-amino-6,7-dihydropyrrolo[3,4-b]pyridin-5-one

A solution of tert-butyl N-tert-butoxycarbonyl-N-(5-oxo-6,7-dihydropyrrolo[3,4-b]pyridin-3-yl)carbamate (592 mg, 1.69 mmol) in TFA (5 mL) and DCM (5 mL) was stirred at rt for 16 hr. The solvents were evaporated to obtain 3-amino-6,7-dihydropyrrolo[3,4-b]pyridin-5-one (0.9 g, crude, CF₃COOH) which was used in the next step without further purification. LCMS(ESI): [M+1]⁺ m/z: calcd 264.2; found 264.2; Rt=0.737 min.

### Step 5. Synthesis of 2,2,2-trifluoroethyl 2-oxo-2-[(5-oxo-6,7-dihydropyrrolo[3,4-b]pyridin-3-yl)amino]acetate

2,2,2-Trifluoroethyl 2-chloro-2-oxo-acetate (651.49 mg, 3.42 mmol) was added dropwise to a solution of 3-amino-6,7-dihydropyrrolo[3,4-b]pyridin-5-one (0.9 g, 3.42 mmol, CF₃COOH) and triethylamine (1.04 g, 10.26 mmol, 1.43 mL) in DCM (20 mL) at 0° C. After addition was complete, cooling bath was removed and resulting mixture was allowed to warm up to 20° C. and stirred for 18 hr. Then, it was washed with water (2×10 ml) and the combined organic fractions was concentrated under reduced pressure to afford crude product (with TFA*Et3N). QA showed that the reaction gave a hydrolysis product which was purified by column chromatography (Interchim; 40 g SiO2, MTBE-MeOH from 0˜100%, flow rate=50 mL/min, cv=19) to afford 2-oxo-2-[(5-oxo-6,7-dihydropyrrolo[3,4-b]pyridin-3-yl)amino]acetic acid (130 mg, 587.79 μmol, 17.19% yield). LCMS(ESI): [M+1]⁺ m/z: calcd 222.0; found 222.0; Rt=0.389 min.

### Step 6. Synthesis of 2-oxo-2-[(5-oxo-6,7-dihydropyrrolo[3,4-b]pyridin-3-yl)amino]acetic acid

To a solution of 2,2,2-trifluoroethyl 2-oxo-2-[(5-oxo-6,7-dihydropyrrolo[3,4-b]pyridin-3-yl)amino]acetate in MeOH (5 mL) was added Lithium hydroxide monohydrate, 98% and the resulting mixture was left to stir at rt for 16 hr. Then the resulting mixture was evaporated to dryness to give 2-oxo-2-[(5-oxo-6,7-dihydropyrrolo[3,4-b]pyridin-3-yl)amino]acetic acid.

### Intermediate 36. 2-oxo-2-((5-oxo-5,6,7,8-tetrahydro-1,6-naphthyridin-3-yl)amino) acetic acid

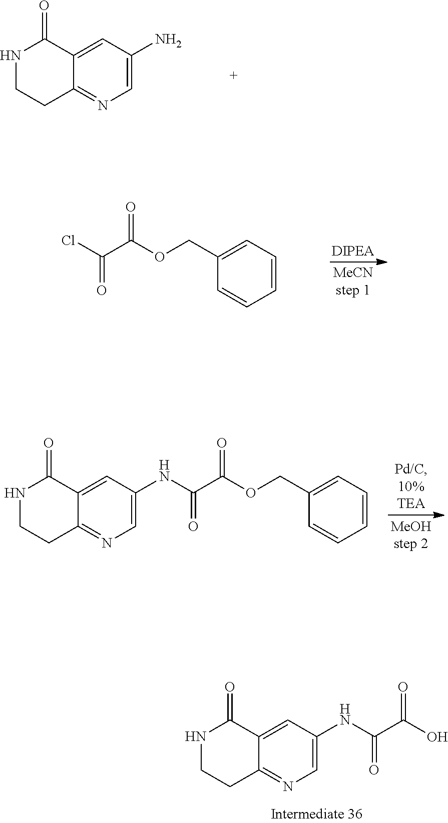 [SMILES: Nc1cnc2c(c1)C(=O)NCC2 ; O=C(Cl)C(=O)OCc1ccccc1 ; O=C(Nc1cnc2c(c1)C(=O)NCC2)C(=O)OCc1ccccc1 ; O=C(O)C(=O)Nc1cnc2c(c1)C(=O)NCC2]

### Step 1: Synthesis of benzyl 2-oxo-2-((5-oxo-5,6,7,8-tetrahydro-1,6-naphthyridin-3-yl)amino)acetate

Benzyl 2-chloro-2-oxo-acetate (6.67 g, 33.57 mmol) was added dropwise to an ice-cooled solution of 3-amino-7,8-dihydro-6H-1,6-naphthyridin-5-one (4.98 g, 30.52 mmol) and DIPEA (4.73 g, 36.62 mmol, 6.38 mL) in MeCN (100 mL). After addition was complete, cooling bath was removed and resulting mixture was stirred at 20° C. for 15 hr. Then, volatiles were distilled off under reduced pressure and residue was triturated with water (50 ml). Resulting brown solid was filtered, rinsed with hexanes (30 ml) and dried, affording benzyl 2-oxo-2-[(5-oxo-7,8-dihydro-6H-1,6-naphthyridin-3-yl)amino]acetate (9.4 g, 28.89 mmol, 94.68% yield). ¹H NMR (600 MHz, DMSO-d₆) δ (ppm) 2.98 (t, 2H), 3.42 (t, 2H), 5.32 (s, 2H), 7.42 (m, 5H), 8.12 (s, 1H), 8.53 (s, 1H), 8.88 (s, 1H), 11.15 (s, 1H). LCMS(ESI): [M]⁺ m/z: calcd 325.2; found 326.2; Rt=0.912 min.

### Step 2: Synthesis of 2-oxo-2-((5-oxo-5,6,7,8-tetrahydro-1,6-naphthyridin-3-yl)amino) acetic acid

Palladium, 10% on carbon. (1 g, 939.67 μmol, 10% purity) was added to a suspension of benzyl 2-oxo-2-[(5-oxo-7,8-dihydro-6H-1,6-naphthyridin-3-yl)amino]acetate (9.4 g, 28.89 mmol) in MeOH (150 mL) and TEA (5.85 g, 57.79 mmol, 8.05 mL). Resulting mixture was stirred under hydrogen atmosphere (balloon pressure) at 40° C. for 16 hr. Then, catalyst was filtered off and filtrate was acidified with 4N diox/HCl to pH≈3. Resulting grey precipitate was filtered and dried, affording 2-oxo-2-[(5-oxo-7,8-dihydro-6H-1,6-naphthyridin-3-yl)amino]acetic acid (6.45 g, 27.42 mmol, 94.91% yield). ¹H NMR (600 MHZ, DMSO-d₆) δ (ppm) 2.96 (t, 2H), 3.40 (t, 2H), 8.11 (s, 1H), 8.55 (s, 1H), 8.88 (s, 1H), 11.02 (s, 1H).

LCMS(ESI): [M]⁺ m/z: calcd 235.4; found 236.2; Rt=0.410 min.

### Intermediate 37. 2-oxo-2-[[1-(2-trimethylsilylethoxymethyl)pyrazolo[4,3-c]pyridin-7-yl]amino]acetic acid

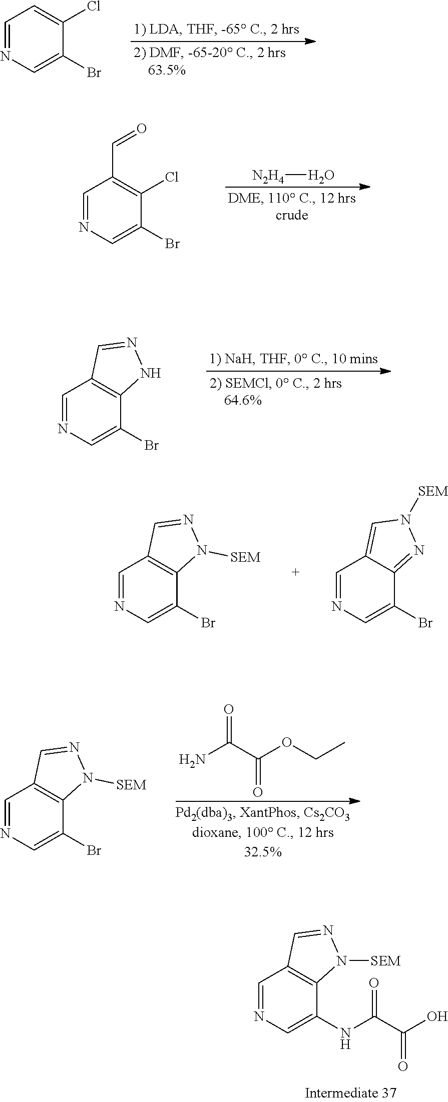

### Step 1: Synthesis of 5-bromo-4-chloro-pyridine-3-carbaldehyde

To a mixture of 2M LDA/THF (35 mL, 70.0 mmol) in THF (20 mL) was added 3-bromo-4-chloro-pyridine (11 g, 57.2 mmol) dropwise at −65° C. under N₂. The resulting mixture was stirred at −65° C. for 2 hours. N,N-dimethylformamide (5.4 mL, 69.7 mmol) was added at −65° C. and the reaction was stirred at 20° C. for 2 hours. The resulting mixture was quenched by addition of NH₄Cl aqueous solution (100 mL) and extracted with EtOAc (100 mL*3). The combined organic layer was washed with saturated NH₄Cl aqueous solution (100 mL), brine (100 mL), dried over anhydrous Na₂SO₄, filtered and concentrated under reduced pressure. The residue was purified by flash chromatography (ISCO®; 80 g AgelaFlash® Silica Flash Column, petroleum ether/EtOAc with EtOAc from 0˜30%, flow rate=30 mL/min, 254 nm) to afford 5-bromo-4-chloro-pyridine-3-carbaldehyde (8 g, 63.5% yield) as yellow solid. ¹H NMR (400 MHZ, DMSO-d₆) δ ppm 10.22-10.40 (m, 1H), 9.02-9.18 (m, 1H), 8.83-8.95 (m, 1H); LCMS(ESI) [M+H]⁺ m/z: calcd 221.9, found 221.9.

### Step 2: Synthesis of 7-bromo-1H-pyrazolo[4,3-c]pyridine

A mixture of 5-bromo-4-chloro-pyridine-3-carbaldehyde (6.1 g, 27.7 mmol) in hydrazine (10 mL, 17.0 mol) and DME (40 mL) was stirred at 110° C. for 12 hours. The resulting mixture was filtered and concentrated under reduced pressure to give 7-bromo-1H-pyrazolo[4,3-c]pyridine (5.6 g, crude) as yellow solid. ¹H NMR (400 MHZ, methanol-d₄) δ ppm 9.05 (s, 1H), 8.42 (d, J=13.1 Hz, 2H).

### Step 3: Synthesis of 2-[(7-bromopyrazolo[4,3-c]pyridin-1-yl)methoxy]ethyl-trimethyl-silane and 2-[(7-bromopyrazolo[4,3-c]pyridin-2-yl)methoxy]ethyl-trimethyl-silane

To a mixture of 7-bromo-1H-pyrazolo[4,3-c]pyridine (5.6 g, 28.3 mmol) in DMF (60 mL) was added NaH (2 g, 50.0 mmol, 60 wt % in mineral oil) slowly at 0° C., the mixture was stirred at 0° C. for 10 minutes. Then 2-(chloromethoxy)ethyl-trimethyl-silane (5.6 mL, 31.6 mmol) was added dropwise. The resulting mixture was stirred at 0° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 40 g AgelaFlash® Silica Flash Column, petroleum ether/EtOAc with EtOAc from 0˜15%, flow rate=50 mL/min, 254 nm) to afford P1 and P2.
P1: 2-[(7-bromopyrazolo[4,3-c]pyridin-1-yl)methoxy]ethyl-trimethyl-silane (6 g, 64.6% yield) was obtained as yellow oil. ¹H NMR (400 MHZ, methanol-d₄) δ ppm 9.05 (s, 1H), 8.53 (s, 1H), 8.37 (s, 1H), 6.06 (s, 2H), 3.64 (t, J=7.9 Hz, 2H), 0.85 (t, J=7.9 Hz, 2H), −0.09 (s, 9H); LCMS(ESI) [M+H]⁺ m/z: calcd 330.0, found 329.9.
P2: 2-[(7-bromopyrazolo[4,3-c]pyridin-2-yl)methoxy]ethyl-trimethyl-silane (1 g, 10.8% yield) as yellow oil. LCMS(ESI) [M+H]⁺ m/z: calcd 330.0, found 329.9.

### Step 4: Synthesis of 2-oxo-2-[[1-(2-trimethylsilylethoxymethyl)pyrazolo[4,3-c]pyridin-7-yl]amino]acetic acid

To a mixture of 2-[(7-bromopyrazolo[4,3-c]pyridin-1-yl)methoxy]ethyl-trimethyl-silane (6 g, 18.3 mmol) and ethyl 2-amino-2-oxo-acetate (6.42 g, 54.8 mmol) in dioxane (70 mL) were added Cs₂CO₃ (9.09 g, 27.9 mmol), XantPhos (1.80 g, 3.61 mmol) and Pd₂(dba)₃ (1.68 g, 1.83 mmol). The resulting mixture was sealed and degassed under vacuum and purged with N₂ for three times, and then stirred at 100° C. for 12 hours under N₂ atmosphere. The resulting mixture was quenched by addition of water (50 mL) and extracted with EtOAc (100 mL*2). The combined water layer was concentrated under reduced pressure. The residue was purified by flash chromatography (Biotage®, Column: SepaFlash® Sphercial C18, 60 g, 40-60 μm, 120 Å; MeCN/water (0.05 v % NH₃—H₂O) with MeCN from 0-23%, 25 mL/min, 254 nm) to give 2-oxo-2-[[1-(2-trimethylsilylethoxymethyl)pyrazolo[4,3-c]pyridin-7-yl]amino]acetic acid (2 g, 32.5% yield) as yellow solid. ¹H NMR (400 MHZ, methanol-d₄) δ ppm 8.93 (d, J=13.1 Hz, 2H), 8.31 (s, 1H), 5.92 (s, 2H), 3.56-3.62 (m, 2H), 0.89-0.99 (m, 2H), −0.08 (s, 9H); LCMS(ESI) [M+H]⁺ m/z: calcd 337.1, found 337.0.

### Intermediate 38. 2-[(6-bromoimidazo[4,5-b]pyridin-3-yl)methoxy]ethyl-trimethyl-silane

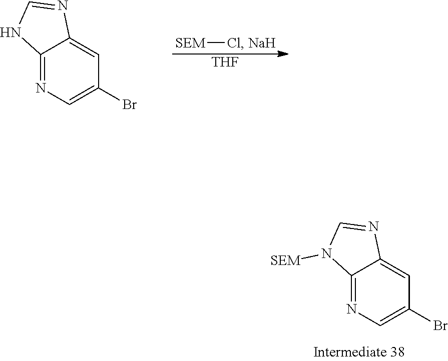

To a stirred solution of 6-bromo-3H-imidazo[4,5-b]pyridine (5 g, 25.25 mmol) in THF (80 mL) at 0° C. (ice-water bath) was added sodium hydride (60% in oil dispersion, 1.51 g, 37.87 mmol) portionwise. The reaction mixture was stirred at 0° C. for 1 hour. After 1 hour, 2-(chloromethoxy)ethyl-trimethyl-silane (4.63 g, 27.77 mmol, 4.92 mL) was added and the resulting reaction mixture was stirred at room temperature for 5 hours. After 5 hours, the reaction mixture was quenched with water (80 mL) and extracted with ethyl acetate. The combined organic layers were washed with brine, dried over Na₂SO₄ and concentrated in vacuo. The obtained residue was purified by column chromatography to get 2-[(6-bromoimidazo[4,5-b]pyridin-3-yl)methoxy]ethyl-trimethyl-silane (2.5 g, 7.62 mmol, 30.16% yield) and 2-[(6-bromoimidazo[4,5-b]pyridin-1-yl)methoxy]ethyl-trimethyl-silane (3.85 g, 11.73 mmol, 46.45% yield). ¹H NMR (CDCl₃, 500 MHZ): δ (ppm) −0.04 (s, 9H), 0.93 (t, 2H), 3.61 (t, 2H), 5.65 (s, 2H), 8.19 (s, 1H), 8.23 (s, 1H), 8.47 (s, 1H).

### Intermediate 39. 7-bromo-4-methyl-1-tetrahydropyran-2-yl-pyrazolo[4,3-c]pyridine

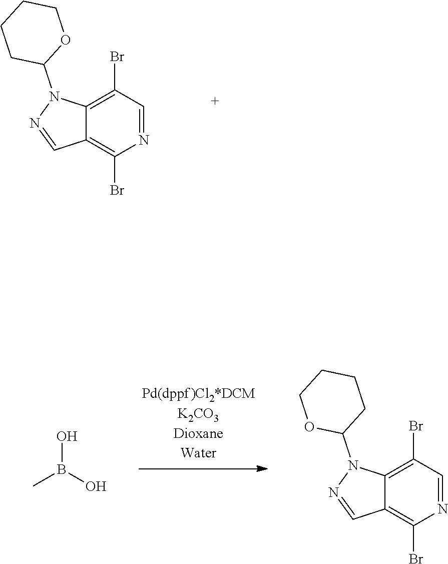

The synthesis of 4,7-dibromo-1-tetrahydropyran-2-yl-pyrazolo[4,3-c]pyridine is given in Example 105

Methylboronic acid (332 mg, 5.54 mmol) was added portion-wise to a solution of 4,7-dibromo-1-tetrahydropyran-2-yl-pyrazolo[4,3-c]pyridine (1.00 g, 2.77 mmol), Pd(dppf)Cl₂ (50.7 mg, 69.3 μmol) and Potassium carbonate-granular (1.91 g, 13.9 mmol, 836 μL) in water (5.00 mL)/dioxane (25.0 mL). The reaction mixture was stirred at 100° C. for 72 hr. The resulting mixture was filtered and concentrated in vacuo. The residue was subjected to CC (Companion; 40 g SiO₂; CHCl₃-MeCN from 0-20%) to afford 7-bromo-4-methyl-1-tetrahydropyran-2-yl-pyrazolo[4,3-c]pyridine (0.36 g, 1.22 mmol, 43.9% yield) as a beige solid. LCMS(ESI): [M+H]⁺ m/z: calcd 296.05 and 298.05; found 298.0; Rt=0.874.

### Intermediate 40. 4-iodo-2,7-naphthyridin-1-amine

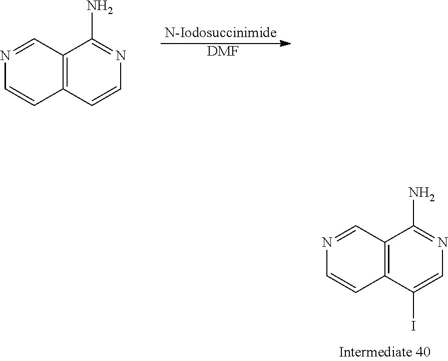

To a solution of 2,7-naphthyridin-1-amine (1.25 g, 8.61 mmol) in DMF (15 mL) was added N-Iodosuccinimide (2.13 g, 9.47 mmol). The resulting mixture was stirred at 20° C. for 16 hr. Then, it was poured onto mixture of water with ice and insoluble material was collected by filtration, filter cake was washed with additional amount of water (˜50 mL) and solid was redissolved in EtOAc (250 mL), dried over Na2SO4 and concentrated on vacuo to obtain 4-iodo-2,7-naphthyridin-1-amine (2.16 g, 7.97 mmol, 92.54% yield). ¹H NMR (400 MHz, DMSO-d₆) δ (ppm) 9.42 (s, 1H), 8.69 (dd, 1H), 8.31 (s, 1H), 7.55 (s, 1H), 7.48 (s, 1H).

LCMS(ESI): [M+1]⁺ m/z: calcd 270.9; found 272.0; Rt=0.788 min.

### Intermediate 41. 5-Iodo-2,7-naphthyridin-1-amine

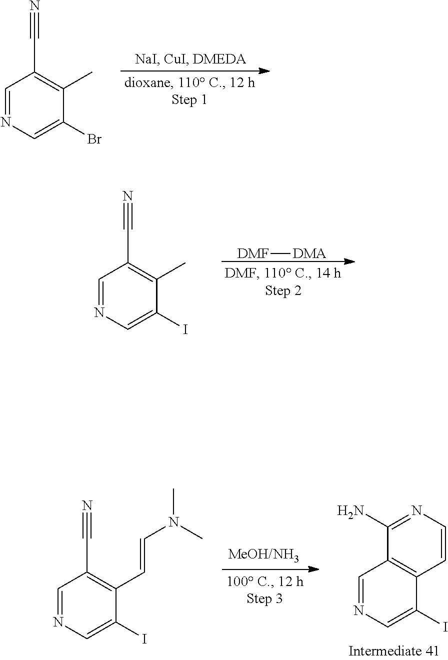

### Step 1: The Synthesis of 5-Iodo-4-methyl-pyridine-3-carbonitrile

To a solution of 5-bromo-4-methyl-pyridine-3-carbonitrile (5 g, 25.38 mmol) in dioxane (100 mL) Copper (I) iodide (241.65 mg, 1.27 mmol, 43.00 μL) and 1,2-dimethylethylenediamine (223.70 mg, 2.54 mmol) were added. The reaction mixture was stirred for 5 min before Sodium iodide (3.99 g, 26.65 mmol, 1.09 mL) was added. Then reaction was stirred overnight at 110° C. Starting substrate was detected in aliquot by LCMS. Sodium iodide (3.80 g, 25.38 mmol, 1.04 mL) was added to reaction and it was stirred overnight at 110° C. After the completion of the reaction, monitored by LCMS, the resulting suspension was concentrated under reduced pressure and water was added and extracted with EtOAc (2*100 ml). The combined organic extracts was dried over Na₂SO₄ and evaporated to afford 5-iodo-4-methyl-pyridine-3-carbonitrile (4 g, 16.39 mmol, 64.59% yield) with 80% purity and it was used in next step without further purification. ¹H NMR (500 MHZ, DMSO-d₆) δ 2.68 (s, 3H), 8.84 (s, 1H), 9.11 (s, 1H). LCMS(ESI): [M+H]⁺ m/z: calcd 244.0; found 245.0; Rt=1.091 min.

### Step 2: The Synthesis of 4-[(E)-2-(Dimethylamino) vinyl]-5-iodo-pyridine-3-carbonitrile

5-Iodo-4-methyl-pyridine-3-carbonitrile (4 g, 16.39 mmol) was dissolved in DMF (50 mL) and dimethylformamide-dimethylacetal (3.91 g, 32.78 mmol, 4.39 mL) was added. Then reaction mixture was heated at 110° C. for 14 hr. Solvent was evaporated and crude was washed with hot MTBE. After that MTBE was evaporated in vacuo to afford 4-[(E)-2-(dimethylamino) vinyl]-5-iodo-pyridine-3-carbonitrile (4 g, 13.37 mmol, 81.59% yield). 1H NMR (500 MHz, DMSO-d₆) δ 3.02 (s, 6H), 5.08 (d, 1H), 7.72 (d, 1H), 8.52 (s, 1H), 8.86 (s, 1H).

### Step 3: The Synthesis of 5-Iodo-2,7-naphthyridin-1-amine

4-[(E)-2-(Dimethylamino) vinyl]-5-iodo-pyridine-3-carbonitrile (1 g, 3.34 mmol) with MeOH/NH₃ (3.34 mmol) in autoclave were stirred overnight at 100° C. Then resulting crude was washed with MeOH to afford to 5-iodo-2,7-naphthyridin-1-amine (0.1 g, 368.93 μmol, 11.03% yield). ¹H NMR (400 MHZ, CDCl₃) δ 6.79 (s, 1H), 7.79 (m, 2H), 8.12 (s, 1H), 8.92 (s, 1H), 9.48 (s, 1H). LCMS(ESI): [M+2H]⁺ m/z: calcd 270.1; found 272.0; Rt=0.719 min.

### Intermediate 42. 2-oxo-2-[[2-(2-trimethylsilylethoxymethyl)pyrazolo[3,4-c]pyridin-4-yl]amino]acetic acid

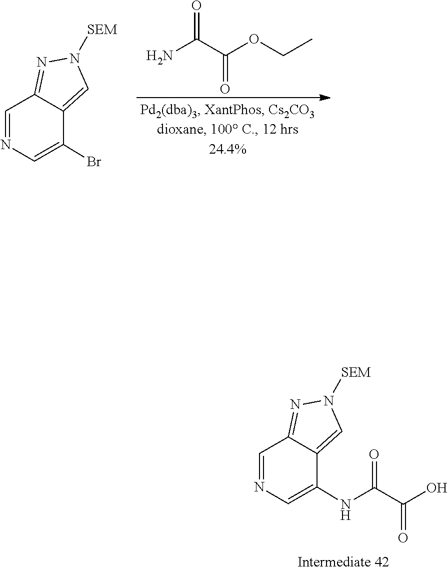

### Step 1: Synthesis of 2-oxo-2-[[2-(2-trimethylsilylethoxymethyl)pyrazolo[3,4-c]pyridin-4-yl]amino]acetic acid

To a mixture of 2-[(4-bromopyrazolo[3,4-c]pyridin-2-yl)methoxy]ethyl-trimethyl-silane (1 g, 3.05 mmol) (see Intermediate 37) and ethyl 2-amino-2-oxo-acetate (1.07 g, 9.14 mmol) in dioxane (10 mL) were added Cs₂CO₃ (1.99 g, 6.09 mmol), XantPhos (300 mg, 0.602 mmol) and Pd₂(dba)₃ (280 mg, 0.306 mmol). The resulting mixture was degassed under vacuum and purged with N₂ for three times, and then stirred at 100° C. for 12 hours under N₂ atmosphere. The resulting mixture was quenched by addition of water (50 mL) and extracted with EtOAc (100 mL*2). The combined water layer was concentrated under reduced pressure. The residue was purified by flash chromatography (Column: SepaFlash® Sphercial C18, 40 g, 40-60 μm, 120 Å; MeCN/water (0.05 v % NH₃—H₂O) with MeCN from 0-30%, 25 mL/min, 254 nm) to afford 2-oxo-2-[[2-(2-trimethylsilylethoxymethyl)pyrazolo[3,4-c]pyridin-4-yl]amino]acetic acid (250 mg, 24.4% yield) as yellow solid. LCMS(ESI) [M+H]⁺ m/z: calcd 337.1, found 337.1.

### Intermediate 43. (S)-2-(1,4-dimethylpiperidin-4-yl)-5-(5-methyl-3,4,5,6-tetrahydropyridin-2-yl)benzo[d]thiazole

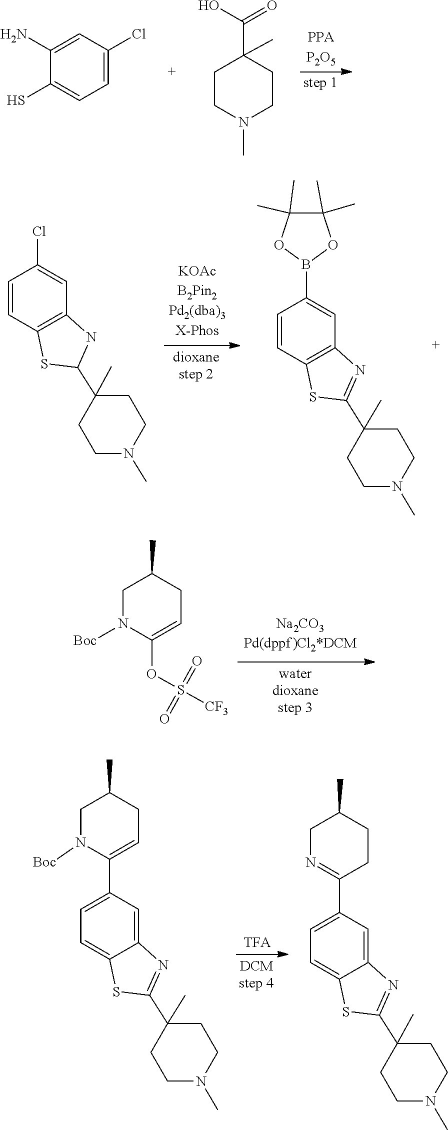

### Step 1: Synthesis of 5-chloro-2-(1,4-dimethylpiperidin-4-yl)benzo[d]thiazoleamine

Prepared by general procedure scheme S2 step 1A. Yield: 3 g (41.38%).

CC conditions: The crude product was purified by silica gel with MeCN/MeOH (gradient 10-100% MeOH) as an eluent mixture.

LCMS(ESI): [M]⁺ m/z: calcd 280.2; found 281.2; Rt=1.529 min.

### Step 2: Synthesis of 2-(1,4-dimethylpiperidin-4-yl)-5-(4,4,5,5-tetramethyl-1,3,2-dioxaborolan-2-yl)benzo[d]thiazole

tris(Dibenzylideneacetone)dipalladium(0) (652.18 mg, 712.21 μmol) and XPhos (1.36 g, 2.85 mmol) was added to a solution of 5-chloro-2-(1,4-dimethyl-4-piperidyl)-1,3-benzothiazole (4 g, 14.24 mmol) and 4,4,5,5-tetramethyl-2-(4,4,5,5-tetramethyl-1,3,2-dioxaborolan-2-yl)-1,3,2-dioxaborolane (4.70 g, 18.52 mmol) in dioxane (60.00 mL). Reaction flask was evacuated and refilled with argon 3 times. Then potassium acetate (2.80 g, 28.49 mmol, 1.78 mL) was added under stream of argon. Resulting mixture was stirred at 100° C. for 15 hr under inert atmosphere, then cooled and evaporated in vacuum poured into water (120 ml) and extracted with DCM (2×50 ml), dried over sodium sulphate and evaporated in vacuum to afford 2-(1,4-dimethyl-4-piperidyl)-5-(4,4,5,5-tetramethyl-1,3,2-dioxaborolan-2-yl)-1,3-benzothiazole (0.9 g, 2.42 mmol, 16.97% yield).

LCMS(ESI): [M]⁺ m/z: calcd 372.2; found 373.2; Rt=3.119 min.

### Step 3: Synthesis of (S)-tert-butyl 6-(2-(1,4-dimethylpiperidin-4-yl)benzo[d]thiazol-5-yl)-3-methyl-3,4-dihydropyridine-1(2H)-carboxylate

Prepared by general procedure scheme S2 step 3. Yield: 1.3 g of crude.

LCMS(ESI): [M]⁺ m/z: calcd 441.2; found 442.2; Rt=1.289 min.

### Step 4: Synthesis of (S)-2-(1,4-dimethylpiperidin-4-yl)-5-(5-methyl-3,4,5,6-tetrahydropyridin-2-yl)benzo[d]thiazole

Prepared by general procedure scheme S2 step 4. Yield: 0.7 g of crude.

LCMS(ESI): [M]⁺ m/z: calcd 341.2; found 342.2; Rt=0.724 min.

### Intermediate 44. (R)—N,N-dimethyl-1-(3-((2R,5S)-5-methylpiperidin-2-yl)phenoxy)propan-2-amine

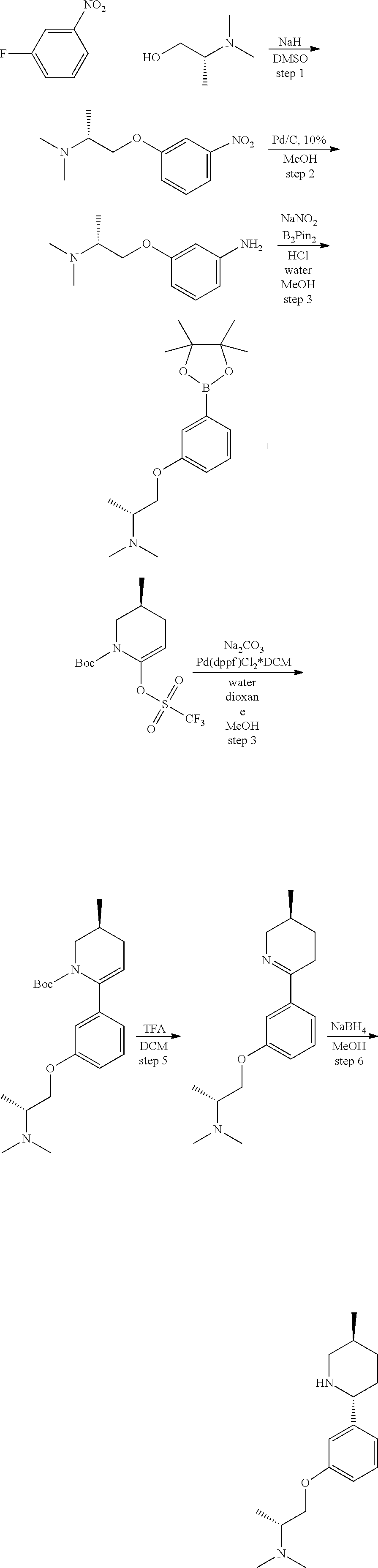

### Step 1: Synthesis of (R)—N,N-dimethyl-1-(3-nitrophenoxy)propan-2-amine)

Sodium hydride (in oil dispersion) 60% dispersion in mineral oil (3.70 g, 96.67 mmol, 60% purity) was added to DMSO (90 mL) and the resulting mixture was stirred for 30 min. A solution of (2R)-2-(dimethylamino) propan-1-ol (9.07 g, 87.88 mmol) in DMSO (10 mL) was added dropwise to the previous mixture and the resulting mixture was stirred for 30 min. 1-Fluoro-3-nitro-benzene (12.4 g, 87.88 mmol, 9.36 mL) was added dropwise to the previous mixture and the resulting mixture was stirred for 1.5 hr. The reaction mixture was quenched with aq. NH₄Cl (100 ml) and the resulting mixture was extracted with EtOAc (3*100 ml). Combined organic layers were washed with brine (3*80 ml), dried over Na₂SO₄, filtered and concentrated in vacuum. The residue was purified by flash chromatography (gradient MeOH in MTBE from 0% to 50%) to obtain (2R)—N,N-dimethyl-1-(3-nitrophenoxy)propan-2-amine (13.7 g, 61.09 mmol, 69.52% yield).

LCMS(ESI): [M]⁺ m/z: calcd 224.2; found 225.2; Rt=0.486 min.

### Step 2: Synthesis of (R)-3-(2-(dimethylamino)propoxy)aniline

(2R)—N,N-Dimethyl-1-(3-nitrophenoxy)propan-2-amine (13.7 g, 61.09 mmol) was dissolved in MeOH (280 mL) and palladium, 10% on carbon, Type 487, dry (1.30 g, 12.22 mmol) was added thereto. The resulting mixture was evacuated and backfilled three times with hydrogen. The reaction mixture was hydrogenated at 1 atm (balloon) overnight. 0.5 g of 10% Pd/C was added to the reaction mixture and the resulting mixture was evacuated and backfilled three times with hydrogen. The reaction mixture was hydrogenated at 1 atm (balloon) overnight. The catalyst was filtered off and the filtrate was concentrated in vacuum to obtain 3-[(2R)-2-(dimethylamino)propoxy]aniline (11.44 g, crude).

LCMS(ESI): [M]⁺ m/z: calcd 194.2; found 195.2; Rt=0.155 min.

### Step 3: Synthesis of (R)—N,N-dimethyl-1-(3-(4,4,5,5-tetramethyl-1,3,2-dioxaborolan-2-yl)phenoxy)propan-2-amine

3-[(2R)-2-(Dimethylamino)propoxy]aniline (11.4 g, 58.68 mmol) was dissolved in MeOH (125 mL) and hydrochloric acid, 36% w/w aq. soln. (17.83 g, 176.04 mmol, 14.86 mL, 36% purity) was added thereto followed by the addition of water (100 mL). The resulting mixture was cooled to 0° C. in an ice bath and a solution of sodium nitrite (4.45 g, 64.55 mmol, 2.05 mL) in water (25 mL) was added dropwise at 0° C. After addition completed, the resulting mixture was stirred for 30 min. A solution of bis(pinacolato)diboron (19.37 g, 76.28 mmol) was added to the previous mixture and the resulting mixture was stirred for 14 hr. The reaction mixture was diluted with water (250 ml) and the resulting mixture was extracted with DCM (3*150 ml). The aqueous layer was basified with NaHCO₃ and extracted with DCM (3*200 ml). Combined organic layers were dried over Na₂SO₄, filtered and concentrated in vacuum to obtain (2R)—N,N-dimethyl-1-[3-(4,4,5,5-tetramethyl-1,3,2-dioxaborolan-2-yl)phenoxy]propan-2-amine (10.7 g, crude).

LCMS(ESI): [M]⁺ m/z: calcd 305.2; found 306.2; Rt=0.955 min.

### Step 4: Synthesis of (S)-tert-butyl 6-(3-((R)-2-(dimethylamino)propoxy)phenyl)-3-methyl-3,4-dihydropyridine-1(2H)-carboxylate

Prepared by general procedure scheme S8 step 2. Yield: 1.85 g (14.09%).

CC conditions: The crude product was purified by silica gel with MTBE/MeOH as an eluent mixture.

LCMS(ESI): [M]⁺ m/z: calcd 374.2; found 375.2; Rt=0.906 min.

### Step 5: Synthesis of (R)—N,N-dimethyl-1-(3-((S)-5-methyl-3,4,5,6-tetrahydropyridin-2-yl)phenoxy)propan-2-amine

Prepared by general procedure scheme S8 step 3. Yield: 0.95 g of crude.

¹H NMR (400 MHZ, CDCl₃) δ (ppm) 0.96 (d, 3H), 1.21 (m, 4H), 1.36 (m, 1H), 1.71 (m, 1H), 1.87 (m, 1H), 2.42 (s, 6H), 2.58 (m, 1H), 2.74 (m, 1H), 3.08 (m, 1H), 3.24 (m, 1H), 4.05 (m, 2H), 6.92 (m, 1H), 7.26 (m, 2H), 7.37 (m, 1H).

### Step 6: Synthesis of (R)—N,N-dimethyl-1-(3-((2R,5S)-5-methylpiperidin-2-yl)phenoxy)propan-2-amine

Prepared by general procedure scheme S8 step 4. Yield: 850 mg of crude.

¹H NMR (500 MHz, CDCl₃) δ (ppm) 0.90 (d, 3H), 1.19 (m, 6H), 1.86 (m, 4H), 2.43 (s, 6H), 3.08 (m, 2H), 3.59 (m, 1H), 3.96 (m, 2H), 6.82 (m, 1H), 6.90 (m, 1H), 7.05 (m, 1H), 7.21 (m, 1H).

### N,N-dimethyl-1-(5-((S)-5-methyl-3,4,5,6-tetrahydropyridin-2-yl)benzo[d]thiazol-2-yl)ethanamine

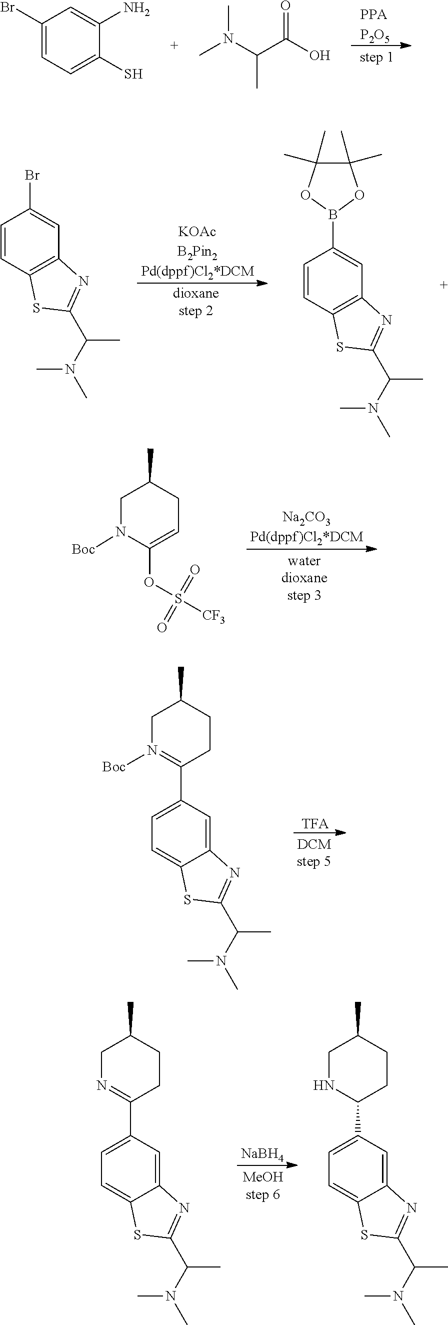

### Step 1: Synthesis of 1-(5-bromobenzo[d]thiazol-2-yl)-N,N-dimethylethanamine

Prepared by general procedure scheme S2 step 1A. Yield: 2.5 g (89.45%).

LCMS(ESI): [M]⁺ m/z: calcd 285.2; found 286.2; Rt=0.759 min.

### Step 2: Synthesis of N,N-dimethyl-1-(5-(4,4,5,5-tetramethyl-1,3,2-dioxaborolan-2-yl)benzo[d]thiazol-2-yl)ethanamine

Prepared by general procedure scheme S2 step 2. Yield: 2.9 g (99.57%).

LCMS(ESI): [M]⁺ m/z: calcd 332.2; found 333.2; Rt=1.123 min.

### Step 3: Synthesis of (3S)-tert-butyl 6-(2-(1-(dimethylamino)ethyl)benzo[d]thiazol-5-yl)-3-methyl-3,4-dihydropyridine-1(2H)-carboxylate

Prepared by general procedure scheme S2 step 3. Yield: 4 g of crude.

LCMS(ESI): [M]⁺ m/z: calcd 401.2; found 402.2; Rt=1.198 min.

### Step 4: Synthesis of N,N-dimethyl-1-(5-((S)-5-methyl-3,4,5,6-tetrahydropyridin-2-yl)benzo[d]thiazol-2-yl)ethanamine

Prepared by general procedure scheme S2 step 4. Yield: 4 g of crude.

LCMS(ESI): [M]⁺ m/z: calcd 301.2; found 302.2; Rt=0.560 min.

### Step 5: Synthesis of N,N-dimethyl-1-(5-((2R,5S)-5-methylpiperidin-2-yl)benzo[d]thiazol-2-yl)ethanamine

Prepared by general procedure scheme S2 step 5. Yield: 4 g of crude.

LCMS(ESI): [M]⁺ m/z: calcd 303.2; found 304.2; Rt=0.509 min.

### 2-(1-methyl-1,2,3,6-tetrahydropyridin-4-yl)-5-((2R,5S)-5-methylpiperidin-2-yl)benzo[d]thiazole

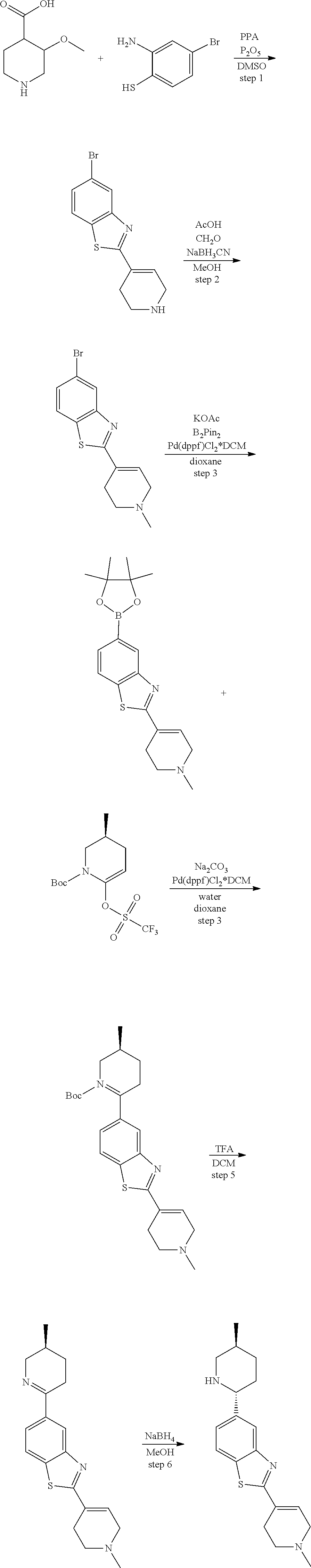

### Step 1: Synthesis of 5-bromo-2-(1,2,3,6-tetrahydropyridin-4-yl)benzo[d]

Prepared by general procedure scheme S2 step 1A. Yield: 2.5 g of crude.

LCMS(ESI): [M]⁺ m/z: calcd 295.2; found 296.2; Rt=0.961 min.

### Step 2: Synthesis of 5-bromo-2-(1-methyl-1,2,3,6-tetrahydropyridin-4-yl)benzo[d]thiazole

Formaldehyde, 37% w/w aq. soln., stab. with 7-8% MeOH (1.03 g, 12.70 mmol, 952.01 μL, 37% purity) and acetic acid (1.02 g, 16.94 mmol, 969.60 μL) were added to a stirred solution of 5-bromo-2-(1,2,3,6-tetrahydropyridin-4-yl)-1,3-benzothiazole (2.5 g, 8.47 mmol) in MeOH (100 mL) at 25° C. The resulting mixture was stirred at 25° C. for 1 hr, then sodium cyan borohydride (638.62 mg, 10.16 mmol) was added in one portion at 25° C. (foaming!). The reaction mixture was stirred at 25° C. for 18 hr, and then concentrated in vacuum. The residue was diluted with 10% aqueous sodium hydroxide solution (70 ml) and extracted with DCM (2*60 ml). The combined organic extracts were dried over sodium sulphate and concentrated in vacuum to afford crude 5-bromo-2-(1-methyl-3,6-dihydro-2H-pyridin-4-yl)-1,3-benzothiazole (2.5 g, 8.08 mmol, 95.46% yield) as light-brown gum, which was used directly in the next step.

LCMS(ESI): [M]⁺ m/z: calcd 309.2; found 310.2; Rt=2.242 min.

### Step 3: Synthesis of 2-(1-methyl-1,2,3,6-tetrahydropyridin-4-yl)-5-(4,4,5,5-tetramethyl-1,3,2-dioxaborolan-2-yl)benzo[d]thiazole

Prepared by general procedure scheme S2 step 2. Yield: 2.88 g of crude.

LCMS(ESI): [M]⁺ m/z: calcd 356.2; found 357.2; Rt=1.131 min.

### Step 4: Synthesis of (S)-tert-butyl 3-methyl-6-(2-(1-methyl-1,2,3,6-tetrahydropyridin-4-yl)benzo[d]thiazol-5-yl)-3,4-dihydropyridine-1(2H)-carboxylate

Prepared by general procedure scheme S2 step 3. Yield: 7 g of crude.

LCMS(ESI): [M]⁺ m/z: calcd 425.2; found 426.2; Rt=1.236 min.

### Step 5: Synthesis of (S)-2-(1-methyl-1,2,3,6-tetrahydropyridin-4-yl)-5-(5-methyl-3,4,5,6-tetrahydropyridin-2-yl)benzo[d]

TFA (29.60 g, 259.60 mmol, 20 mL) was added in one portion to a stirred solution of tert-butyl (3S)-3-methyl-6-[2-(1-methyl-3,6-dihydro-2H-pyridin-4-yl)-1,3-benzothiazol-5-yl]-3,4-dihydro-2H-pyridine-1-carboxylate (7 g, 16.45 mmol) in DCM (20 mL). The resulting solution was stirred at 25° C. for 0.5 hr, and then concentrated in vacuum. The residue was diluted with water (100 ml). The resulting solution of TFA salt of the product was decanted from dark-brown oily residue, which was additionally rinsed with water (2*25 ml). The combined aqueous solution was filtered through a cotton pad to remove traces of oily impurities, then basified to pH 11-12 with 10% aqueous sodium hydroxide solution and extracted with DCM (2*50 ml). The combined organic extracts were dried over sodium sulfate and concentrated in vacuum to afford 2-(1-methyl-3,6-dihydro-2H-pyridin-4-yl)-5-[(3S)-3-methyl-2,3,4,5-tetrahydropyridin-6-yl]-1,3-benzothiazole (2.7 g, 8.30 mmol, 50.44% yield) as brown solid, which was directly used in the next step.

LCMS(ESI): [M]⁺ m/z: calcd 325.2; found 326.2; Rt=0.673 min.

### Step 6: Synthesis of 2-(1-methyl-1,2,3,6-tetrahydropyridin-4-yl)-5-((2R,5S)-5-methylpiperidin-2-yl)benzo[d]thiazole

Prepared by general procedure scheme S2 step 5. Yield: 1.4 g of crude.

LCMS(ESI): [M]⁺ m/z: calcd 327.2; found 328.2; Rt=0.719 min.

### Intermediate 45. rac-2-((2S,5S)-2-(4-fluorophenyl)-4-methoxy-5-methylpiperidin-1-yl)-2-oxoacetamide

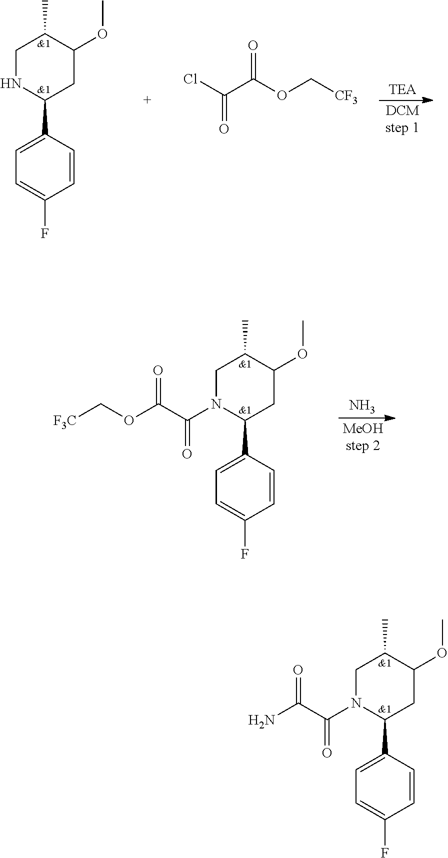

### Step 1: Synthesis of rac-2,2,2-trifluoroethyl 2-((2S,5S)-2-(4-fluorophenyl)-4-methoxy-5-methylpiperidin-1-yl)-2-oxoacetate

2,2,2-Trifluoroethyl 2-chloro-2-oxo-acetate (1.00 g, 5.26 mmol) was added dropwise to a solution of rac-(2S,5S)-2-(4-fluorophenyl)-4-methoxy-5-methyl-piperidine (1.30 g, 5.00 mmol, HCl) and TEA (1.52 g, 15.01 mmol, 2.09 mL) in DCM (25.00 mL) at 0° C. After addition was complete, cooling bath was removed and resulting mixture was allowed to warm up to 20° C. and stirred for 16 hr. Then, it was washed with water, dried over Na₂SO₄ and concentrated under reduced pressure, affording 2,2,2-trifluoroethyl 2-oxo-2-[rac-(2S,5S)-2-(4-fluorophenyl)-4-methoxy-5-methyl-1-piperidyl]acetate (1.7 g, crude) which was used in the next step without further purification.

LCMS(ESI): [M+1]⁺ m/z: calcd 377.2; found 378.2; Rt=1.463 min.

### Step 2: Synthesis of rac-2-((2S,5S)-2-(4-fluorophenyl)-4-methoxy-5-methylpiperidin-1-yl)-2-oxoacetamide

A solution of 2,2,2-trifluoroethyl 2-oxo-2-[rac-(2S,5S)-2-(4-fluorophenyl)-4-methoxy-5-methyl-1-piperidyl]acetate (1.70 g, 4.51 mmol) in MeOH/NH₃ (5N) (25 mL) was stirred at 20° C. for 16 hr. The solvent was evaporated to obtain 2-oxo-2-[rac-(2S,5S)-2-(4-fluorophenyl)-4-methoxy-5-methyl-1-piperidyl]acetamide (1.28 g, crude).

LCMS(ESI): [M+1]⁺ m/z: calcd 294.2; found 295.2; Rt=1.088 min.

### Intermediate 46. 2-(3,5-dichlorophenyl)-5-methyl-piperidine

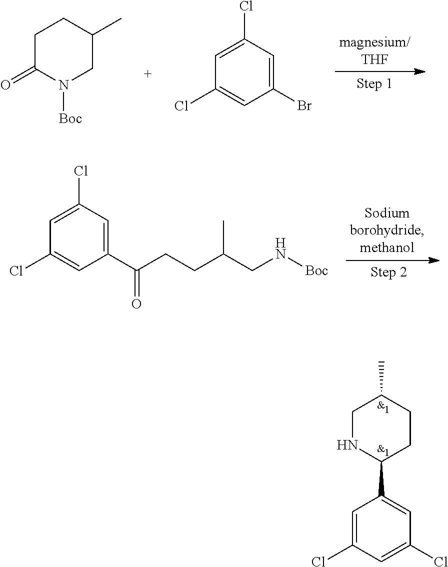 [SMILES: CC1CCC(=O)N(C(=O)OC(C)(C)C)C1 ; Clc1cc(Cl)cc(Br)c1 ; CC(CCC(=O)c1cc(Cl)cc(Cl)c1)CNC(=O)OC(C)(C)C ; C[C@@H]1CC[C@@H](c2cc(Cl)cc(Cl)c2)NC1]

### Step 1. Synthesis of tert-butyl N-[5-(3,5-dichlorophenyl)-2-methyl-5-oxo-pentyl]carbamate

2-[(5-carbamoyl-3-pyridyl)amino]-2-oxo-acetic acid (241.77 mg, 1.16 mmol) and 5-(5-methyl-2-piperidyl)-1H-pyrazolo[4,3-b]pyridine (0.25 g, 1.16 mmol) were mixed in DMF (20 mL). The reaction suspension was cooled to 0° C. and HATU (439.51 mg, 1.16 mmol) followed by TEA (116.97 mg, 1.16 mmol, 161.11 μL) were added and stirred at ambient temperature for 12 hr. The reaction mixture was evaporated in vacuo and obtained crude product 0.48 g was purified by preparative 25-45% 1-6 min water-methanol (NH3 0.1%), flow 30 ml/min to afford product 5-[[2-[(2R,5S)-5-methyl-2-(1H-pyrazolo[4,3-b]pyridin-5-yl)-1-piperidyl]-2-oxo-acetyl]amino]pyridine-3-carboxamide (0.14 g, 343.62 μmol, 29.73% yield).

¹H NMR (400 MHZ, CDCl₃) δ 0.92 (m, 3H), 1.42 (s, 13H), 1.67-1.82 (m, 2H), 2.95 (m, 2H), 4.68 (m, 1H), 7.21 (m, 1H), 7.82 (m, 2H).

LCMS(ESI): [M]⁺ m/z: calcd 360.0; found 360.2; Rt=1.683 min.

### Step 2. Synthesis of 2-(3,5-dichlorophenyl)-5-methyl-piperidine

The tert-butyl N-[5-(3,5-dichlorophenyl)-2-methyl-5-oxo-pentyl]carbamate (12 g, 33.31 mmol) was stirred in Trifluoroacetic acid (18.99 g, 166.54 mmol, 12.83 mL) for 1 hr. TLC was used to check the reaction progress. 50% w/v NaOH solution was added to the mixture until the pH was 13-14. The product was extracted 4×20 mL with DCM and the organic layers combined, dried with MgSO₄ and evaporated. The product was dissolved in mixture water (25 mL)/MeOH (150 mL) and added to a flask followed by Sodium Borohydride (1.26 g, 33.31 mmol, 1.18 mL). The mixture was stirred under Ar overnight. The mixture was acidified with 1-2M HCl until the pH was 1-3 and left for 30 minutes. NaOH solution was then added until the pH was 13-14 and the product was extracted with DCM (4×100 mL), the organic layers were combined, dried with Na₂SO₄, filtered and evaporated. The residue was purified by CC (Interchim, 120 g SiO₂, hexane/MTBE with MTBE from 0˜100%, flow rate=85 ml/min, RV=6.9 CV.) to give 2-(3,5-dichlorophenyl)-5-methyl-piperidine (2.9 g, 11.88 mmol, 35.66% yield) as a light-yellow oil.

¹H NMR (400 MHZ, CDCl₃) δ 0.88 (d, 3H), 1.13 (m, 1H), 1.45 (dd, 1H), 1.63 (m, 2H), 1.77 (d, 1H), 1.85 (d, 1H), 2.38 (dd, 1H), 3.11 (d, 1H), 3.60 (d, 1H), 7.21 (s, 1H), 7.26 (s, 2H).

LCMS(ESI): [M+1]⁺ m/z: calcd 243.0; found 244.2; Rt=0.978 min.

### Intermediate 47. 2-(1,5-dimethylpiperidin-3-yl)-5-((S)-5-methyl-3,4,5,6-tetrahydropyridin-2-yl)benzo[d]thiazole

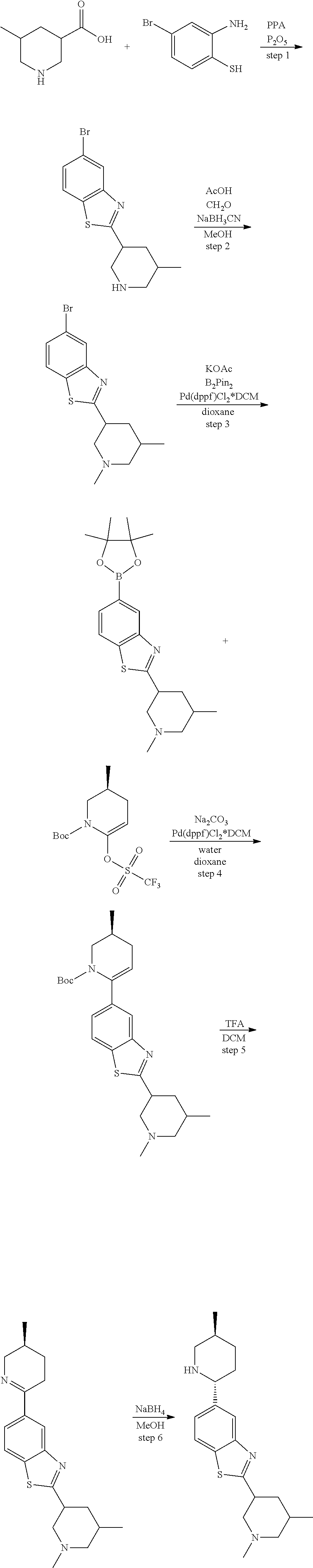

### Step 1: Synthesis of 5-bromo-2-(5-methylpiperidin-3-yl)benzo[d]thiazole

Prepared by general procedure scheme S2 step 1A. Yield: 3.1 g of crude.

LCMS(ESI): [M]⁺ m/z: calcd 311.2; found 312.2; Rt=2.570 min.

### Step 2: Synthesis of 5-bromo-2-(1,5-dimethylpiperidin-3-yl)benzo[d]thiazole

Formaldehyde (808.28 mg, 9.96 mmol, 746.33 μL) (37% solution in water) was added to a stirred solution of 5-bromo-2-(5-methyl-3-piperidyl)-1,3-benzothiazole (3.1 g, 9.96 mmol) and acetic acid (1.20 g, 19.92 mmol, 1.14 mL) in MeOH (28.86 mL), resulting mixture stirred at 25° C. for 1 hr, sodium cyan borohydride (625.92 mg, 9.96 mmol) added in 1 portion and stirred at 25° C. for 18 hr. Reaction mixture concentrated, mixed with NaOH (20% in water, 10 mL), extracted with MTBE (2×20 mL), combined organic layer dried over Na₂SO₄, concentrated to give crude 5-bromo-2-(1,5-dimethyl-3-piperidyl)-1,3-benzothiazole (1.05 g, 3.23 mmol, 32.41% yield).

LCMS(ESI): [M]⁺ m/z: calcd 325.2; found 326.2; Rt=1.038 min.

### Step 3: Synthesis of 2-(1,5-dimethylpiperidin-3-yl)-5-(4,4,5,5-tetramethyl-1,3,2-dioxaborolan-2-yl)benzo[d]thiazole

Prepared by general procedure scheme S2 step 2. Yield: 1.1 g (96.1%).

LCMS(ESI): [M]⁺ m/z: calcd 372.2; found 373.2; Rt=2.772 min.

### Step 4: Synthesis of (3S)-tert-butyl 6-(2-(1,5-dimethylpiperidin-3-yl)benzo[d]thiazol-5-yl)-3-methyl-3,4-dihydropyridine-1(2H)-carboxylate

Prepared by general procedure scheme S2 step 3. Yield: 1.2 g of crude.

LCMS(ESI): [M]⁺ m/z: calcd 441.2; found 442.2; Rt=3.100 min.

### Step 5: Synthesis of 2-(1,5-dimethylpiperidin-3-yl)-5-((S)-5-methyl-3,4,5,6-tetrahydropyridin-2-yl)benzo[d]thiazole

Prepared by general procedure scheme S2 step 4. Yield: 0.6 g of crude.

LCMS(ESI): [M]⁺ m/z: calcd 341.2; found 342.2; Rt=1.535 min.

### Intermediate 48. 2-(2-(3-(2-(dimethylamino)ethoxy)phenyl)-5-methylpiperidin-1-yl)-2-oxo-N-(1H-pyrazolo[4,3-c]pyridin-7-yl)acetamide

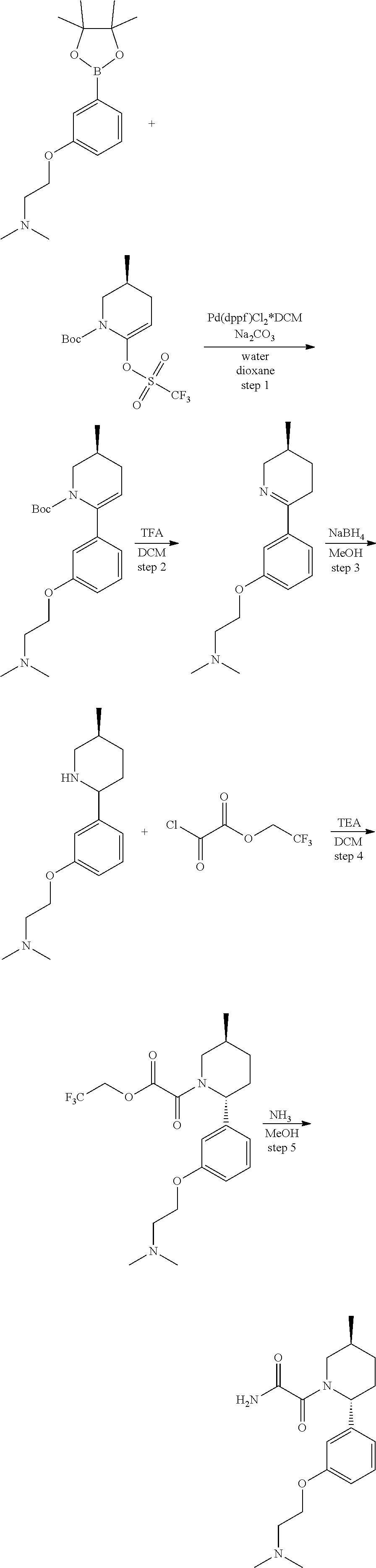

### Step 1: Synthesis of (S)-tert-butyl 6-(3-(2-(dimethylamino)ethoxy)phenyl)-3-methyl-3,4-dihydropyridine-1(2H)-carboxylate

tert-Butyl (3S)-3-methyl-6-(trifluoromethylsulfonyloxy)-3,4-dihydro-2H-pyridine-1-carboxylate (8 g, 23.17 mmol), N,N-dimethyl-2-[3-(4,4,5,5-tetramethyl-1,3,2-dioxaborolan-2-yl)phenoxy]ethanamine (5.62 g, 19.30 mmol) and sodium carbonate (4.09 g, 38.61 mmol, 1.62 mL) were mixed in a mixture of dioxane (45 mL) and water (15 mL). The resulting mixture was evacuated and backfilled three times with argon. Pd(dppf)Cl₂*DCM (788.26 mg, 965.25 μmol) was added to the previous mixture and the resulting mixture was heated at 90° C. overnight. The resulting mixture was cooled and diluted with water (100 ml). The resulting mixture was extracted with EtOAc (2*100 ml) and combined organic layers were washed with brine (60 ml), dried over Na₂SO₄, filtered, and concentrated in vacuum. The residue (10.8 g of crude product) was purified by column chromatography to obtain tert-butyl (3S)-6-[3-[2-(dimethylamino)ethoxy]phenyl]-3-methyl-3,4-dihydro-2H-pyridine-1-carboxylate (4.5 g, 12.48 mmol, 64.66% yield).

LCMS(ESI): [M]⁺ m/z: calcd 360.2; found 361.2; Rt=1.077 min.

### Step 2: Synthesis of (S)—N,N-dimethyl-2-(3-(5-methyl-3,4,5,6-tetrahydropyridin-2-yl)phenoxy)ethanamine

tert-Butyl (3S)-6-[3-[2-(dimethylamino)ethoxy]phenyl]-3-methyl-3,4-dihydro-2H-pyridine-1-carboxylate (4.5 g, 12.48 mmol) was dissolved in DCM (18 mL) and TFA (18 mL) was added thereto. The resulting mixture was stirred for 1 hr. The reaction mixture was carefully poured into aq.NaHCO₃ solution (20 g in 100 ml of water) and the resulting mixture was extracted with DCM (2*50 ml). Combined organic layers were dried over sodium sulfate, filtered, and concentrated in vacuum to obtain N,N-dimethyl-2-[3-[(3S)-3-methyl-2,3,4,5-tetrahydropyridin-6-yl]phenoxy]ethanamine (2.36 g, 9.04 mmol, 72.46% yield).

LCMS(ESI): [M]⁺ m/z: calcd 260.2; found 261.2; Rt=0.245 min.

### Step 3: Synthesis of N,N-dimethyl-2-(3-((5S)-5-methylpiperidin-2-yl)phenoxy)ethanamine

N,N-Dimethyl-2-[3-[(3S)-3-methyl-2,3,4,5-tetrahydropyridin-6-yl]phenoxy]ethanamine (2.36 g, 9.06 mmol) was dissolved in MeOH (45 mL) and sodium borohydride (685.82 mg, 18.13 mmol, 638.57 μL) was added portion wise. The resulting mixture was stirred overnight. Water (20 ml) was added to the reaction mixture and the resulting mixture was concentrated in vacuum. The residue was diluted with water (30 ml) and the resulting mixture was extracted with DCM (2*50 ml). Combined organic layers were dried over Na₂SO₄, filtered, and concentrated in vacuum to obtain N,N-dimethyl-2-[3-[(2R,5S)-5-methyl-2-piperidyl]phenoxy]ethanamine (2.02 g, 7.70 mmol, 84.94% yield).

LCMS(ESI): [M]⁺ m/z: calcd 262.2; found 263.2; Rt=0.498 min.

### Step 4: Synthesis of 2,2,2-trifluoroethyl 2-((2R,5S)-2-(3-(2-(dimethylamino)ethoxy)phenyl)-5-methylpiperidin-1-yl)-2-oxoacetate

N,N-Dimethyl-2-[3-[(2R,5S)-5-methyl-2-piperidyl]phenoxy]ethanamine (1.02 g, 3.89 mmol) and TEA (433.55 mg, 4.28 mmol, 597.17 μL) were dissolved in DCM (25 mL) and the resulting mixture was cooled to −5° C. in an ice/MeOH bath. A solution of 2,2,2-trifluoroethyl 2-chloro-2-oxo-acetate (816.21 mg, 4.28 mmol) in DCM (5 mL) were added dropwise at −5° C. After addition completed, the resulting mixture was allowed to warm to rt and stirred overnight. The reaction mixture was transferred to a separation funnel and washed with aq. NaHCO₃ solution (30 ml) and water (30 ml). The organic layer was dried over Na₂SO₄, filtered, and concentrated in vacuum to obtain 2,2,2-trifluoroethyl 2-oxo-2-[(2R,5S)-2-[3-[2-(dimethylamino)ethoxy]phenyl]-5-methyl-1-piperidyl]acetate (1.28 g, 3.07 mmol, 78.92% yield).

LCMS(ESI): [M]⁺ m/z: calcd 416.2; found 417.2; Rt=0.914 min.

### Step 5: Synthesis of 2-((2R,5S)-2-(3-(2-(dimethylamino)ethoxy)phenyl)-5-methylpiperidin-1-yl)-2-oxoacetamide

2,2,2-Trifluoroethyl 2-oxo-2-[(2R,5S)-2-[3-[2-(dimethylamino)ethoxy]phenyl]-5-methyl-1-piperidyl]acetate (1.28 g, 3.07 mmol) was dissolved in NH₃/MeOH (40 mL) and the resulting mixture was stirred overnight. The reaction mixture was concentrated in vacuum to obtain 2-oxo-2-[(2R,5S)-2-[3-[2-(dimethylamino)ethoxy]phenyl]-5-methyl-1-piperidyl]acetamide (890 mg, 2.67 mmol, 86.84% yield).

LCMS(ESI): [M]⁺ m/z: calcd 333.2; found 334.2; Rt=0.828 min.

### Intermediate 49. 7-((2R,5S)-5-methylpiperidin-2-yl)-2-(1-methylpiperidin-4-yl)quinoline-3-carbonitrile

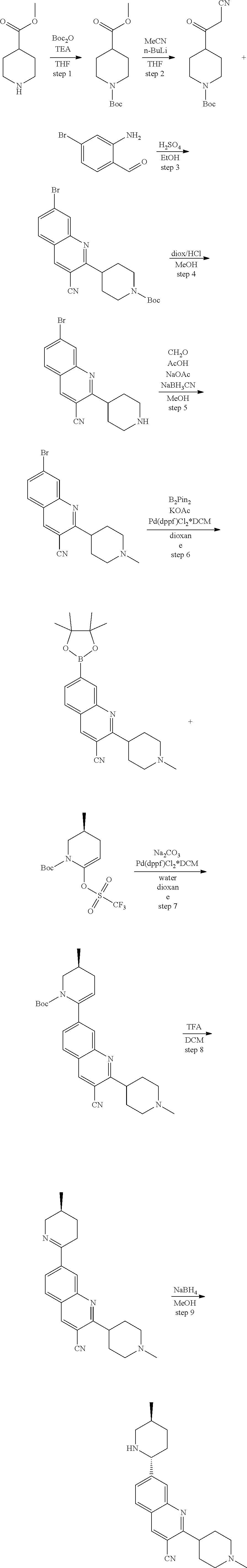

### Step 1: Synthesis of 1-tert-butyl 4-methyl piperidine-1,4-dicarboxylate

To a stirring solution of methyl piperidine-4-carboxylate (15 g, 104.76 mmol, 14.15 mL) in THF (200 mL) was added di-tert-butyl dicarbonate (25.15 g, 115.24 mmol, 26.45 mL), TEA (13.78 g, 136.19 mmol, 18.98 mL) at 0° C. The resulting mixture was stirred at 0° C. for 12 hr, and then evaporated in vacuum. The residue was diluted with water (100 mL) and extracted with DCM (2*100 mL). The combined organic extracts were dried over sodium sulphate and evaporated in vacuum to afford 1-tert-butyl 4-methyl piperidine-1,4-dicarboxylate (22 g, 90.42 mmol, 86.31% yield).

LCMS(ESI): [M−t−Bu]⁺ m/z: calcd 187.2; found 188.2; Rt=3.461 min.

### Step 2: Synthesis of tert-butyl 4-(2-cyanoacetyl)piperidine-1-carboxylate

To a stirring solution of MeCN, 99% (607.43 mg, 14.80 mmol, 772.81 μL) in THF (25 mL) at −78° C. was added n-butyllithium, 2.2M in hexane, packaged under Ar in resalable ChemSeal bottles (947.80 mg, 14.80 mmol, 6 mL) dropwise under argon. The resulting mixture was stirred at −78° C. for 3 hr, and then was added 1-tert-butyl 4-methyl piperidine-1,4-dicarboxylate (3 g, 12.33 mmol) dropwise at −78° C. stirred for 2 hr. The reaction mixture was then stirred for 3 hr at rt, then was added 10% solution NH₄Cl, and was evaporated in vacuum. The residue was diluted with water (100 mL) and extracted with EtOAc (2*30 mL). The combined organic extracts were dried over sodium sulphate and evaporated in vacuum to afford tert-butyl 4-(2-cyanoacetyl)piperidine-1-carboxylate (1.7 g, 6.74 mmol, 54.64% yield).

LCMS(ESI): [M-Boc]⁺ m/z: calcd 152.2; found 153.2; Rt=2.859 min.

### Step 3: Synthesis of tert-butyl 4-(7-bromo-3-cyanoquinolin-2-yl)piperidine-1-carboxylate

tert-Butyl 4-(2-cyanoacetyl)piperidine-1-carboxylate (0.5 g, 1.98 mmol) and 2-amino-4-bromo-benzaldehyde (396.40 mg, 1.98 mmol) was dissolved in EtOH (20 mL) and sulfuric acid (1.94 mg, 19.82 μmol) was added, stirred overnight for 12 hr at 78° C. The reaction mixture was cooled to rt and reaction mixture was filtered to give tert-butyl 4-(7-bromo-3-cyano-2-quinolyl)piperidine-1-carboxylate (0.5 g, 1.20 mmol, 60.61% yield).

LCMS(ESI): [M−t−Bu]⁺ m/z: calcd 360.2; found 361.2; Rt=4.904 min.

### Step 4: Synthesis of 7-bromo-2-(piperidin-4-yl)quinoline-3-carbonitrile

Hydrogen chloride solution 4.0M in dioxane (800.00 mg, 21.94 mmol, 1 mL) was added to a solution of tert-butyl 4-(7-bromo-3-cyano-2-quinolyl)piperidine-1-carboxylate (0.5 g, 1.20 mmol) in MeOH (10 mL). The reaction mixture was stirred at 20° C. for 8 hr, then evaporated and added to MTBE (10 ml) the resulting precipitate was filtered off, washed with MTBE (10 ml) and dried to afford 7-bromo-2-(4-piperidyl)quinoline-3-carbonitrile (0.25 g, 642.48 μmol, 53.49% yield, 2HCl).

LCMS(ESI): [M]⁺ m/z: calcd 316.2; found 317.2; Rt=2.052 min.

### Step 5: Synthesis of 7-bromo-2-(1-methylpiperidin-4-yl)quinoline-3-carbonitrile

Formaldehyde, 37% w/w aq. soln., stab. with 7-8% MeOH (509.35 mg, 16.96 mmol, 470.32 μL) and acetic acid (679.02 mg, 11.31 mmol, 647.31 μL) were added to the solution of 7-bromo-2-(4-piperidyl)quinoline-3-carbonitrile (2.2 g, 5.65 mmol, 2HCl) and sodium acetate, anhydrous (1.39 g, 16.96 mmol, 910.57 μL) in MeOH (20 mL). Resulting mixture was stirred at 0° C. for 1 hr before sodium cyan borohydride (710.57 mg, 11.31 mmol) was added thereto. After that, stirring was continued for 5 hr. Then, solvent was removed under reduced pressure and residue was partitioned between 10% aq. K₂CO₃ solution (40 ml) and DCM (50 ml). Organic layer was separated, dried over solid K₂CO₃ and concentrated under reduced pressure, leaving 7-bromo-2-(1-methyl-4-piperidyl)quinoline-3-carbonitrile (2.1 g, crude).

LCMS(ESI): [M]⁺ m/z: calcd 330.2; found 331.2; Rt=0.831 min.

### Step 6: Synthesis of 2-(1-methylpiperidin-4-yl)-7-(4,4,5,5-tetramethyl-1,3,2-dioxaborolan-2-yl)quinoline-3-carbonitrile

Prepared by general procedure scheme S8 step 1. Yield: 2.5 g of crude.

LCMS(ESI): [M]⁺ m/z: calcd 377.2; found 378.2; Rt=1.061 min.

### Step 7: Synthesis of (S)-tert-butyl 6-(3-cyano-2-(1-methylpiperidin-4-yl)quinolin-7-yl)-3-methyl-3,4-dihydropyridine-1(2H)-carboxylate

Prepared by general procedure scheme S8 step 2. Yield: 3.4 g of crude.

LCMS(ESI): [M]⁺ m/z: calcd 446.2; found 447.2; Rt=2.992 min.

### Step 8: Synthesis of (S)-7-(5-methyl-3,4,5,6-tetrahydropyridin-2-yl)-2-(1-methylpiperidin-4-yl)quinoline-3-carbonitrile

Prepared by general procedure scheme S8 step 3. Yield: 2.2 g of crude.

LCMS(ESI): [M]⁺ m/z: calcd 346.2; found 347.2; Rt=0.567 min.

### Step 9: Synthesis of 7-((2R,5S)-5-methylpiperidin-2-yl)-2-(1-methylpiperidin-4-yl)quinoline-3-carbonitrile

Prepared by general procedure scheme S8 step 4. Yield: 0.7 g of crude.

LCMS(ESI): [M]⁺ m/z: calcd 348.2; found 349.2; Rt=1.864 min.

### Intermediate 50. 2-(3-chloro-4-fluoro-phenyl)-5-methyl-piperidine

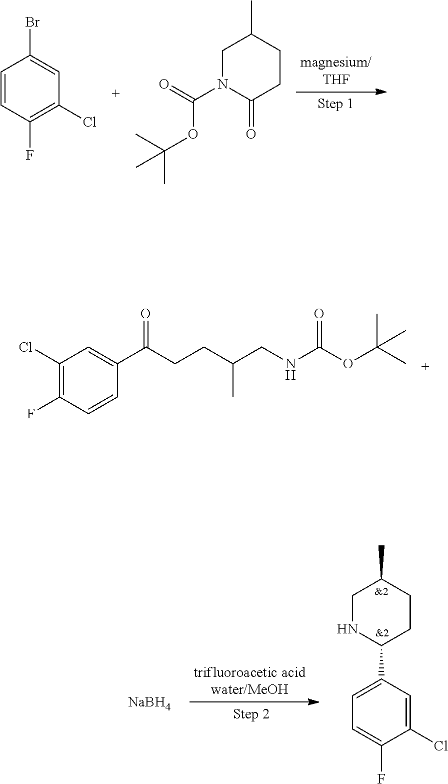 [SMILES: Fc1ccc(Br)cc1Cl ; CC1CCC(=O)N(C(=O)OC(C)(C)C)C1 ; CC(CCC(=O)c1ccc(F)c(Cl)c1)CNC(=O)OC(C)(C)C ; "NaBH4" ; C[C@H]1CC[C@H](c2ccc(F)c(Cl)c2)NC1]

### Step 1: Synthesis of tert-butyl N-[5-(3-chloro-4-fluoro-phenyl)-2-methyl-5-oxo-pentyl]carbamate

To a dry 2 necked flask was added magnesium (1.16 g, 47.75 mmol, 666.93 μL), dry THF (75 mL) and 4-bromo-2-chloro-1-fluoro-benzene (10 g, 47.75 mmol, 5.78 mL) with stirring under Ar. The mixture was heated gently until it maintained its own reflux. When reflux had subsided external heating was applied to maintain reflux for a further 1 hour. tert-butyl 5-methyl-2-oxo-piperidine-1-carboxylate (7.83 g, 36.73 mmol) was added to a dry 3 necked round bottomed flask with a thermometer. Dry THF (75 mL) was added with stirring under Ar and the solution was cooled to −78° C. The Grignard reagent was added to the t-Boc-lactam over 1 hour, maintaining the internal temperature below −70° C. The solution was warmed to room temperature and sat. NH₄Cl was added. The aqueous layer was extracted 3×50 mL with DCM and the organic layers combined, dried over Na₂SO₄, filtered and concentrated in vacuo. tert-butyl N-[5-(3-chloro-4-fluoro-phenyl)-2-methyl-5-oxo-pentyl]carbamate (13 g, crude) was obtained as a light-yellow oil and was used in the next step without further purification.

¹H NMR (CDCl₃, 500 MHz): δ 0.95 (d, 3H), 1.44 (s, 9H), 1.71-1.80 (m, 4H), 2.98 (t, 2H), 4.02 (t, 1H), 4.68 (brs, 1H), 7.20 (t, 1H), 7.87 (dd, 1H), 8.03 (d, 1H).

LCMS(ESI): [M+Na]⁺ m/z: calcd 343.2; found 366.2; Rt=1.622 min.

### Step 2: Synthesis of 2-(3-chloro-4-fluoro-phenyl)-5-methyl-piperidine

The tert-butyl N-[5-(3-chloro-4-fluoro-phenyl)-2-methyl-5-oxo-pentyl]carbamate (13 g, 37.81 mmol) was stirred in trifluoroacetic acid (17.25 g, 151.24 mmol, 11.65 mL) for 1 hr. TLC was used to check the reaction progress. 50% w/v NaOH solution was added to the mixture until the pH was 13-14. The product was extracted 4×20 mL with DCM and the organic layers combined, dried with MgSO₄ and evaporated. The product was dissolved in mixture water (25 mL)/MeOH (150 mL) and added to a flask followed by Sodium Borohydride (1.43 g, 37.81 mmol, 1.34 mL). The mixture was stirred under Ar overnight. The mixture was acidified with 1-2M HCl until the pH was 1-3 and left for 30 minutes. NaOH solution was then added until the pH was 13-14 and the product was extracted with DCM (4×100 mL), the organic layers were combined, dried with Na₂SO₄, filtered and evaporated. The residue was purified by CC (Companion combiflash; 120 g SiO₂; chloroform/acetonitrile with acetonitrile from 0 to 17%, flow rate=85 ml/min, Rv=7-11 cv.) to give 2-(3-chloro-4-fluoro-phenyl)-5-methyl-piperidine (2.2 g, 9.66 mmol, 25.55% yield) as a light-yellow oil.

¹H NMR (CDCl₃, 400 MHZ): δ 0.86 (d, 3H), 1.12 (m, 1H), 1.42 (m, 1H), 1.61-1.84 (m, 4H), 2.37 (t, 1H), 3.10 (d, 1H), 3.48 (d, 1H), 7.04 (t, 1H), 7.18 (dd, 1H), 7.42 (d, 1H).

LCMS(ESI): [M+H]⁺ m/z: calcd 227.1; found 228.2; Rt=0.895 min.

### Example 246. Cellular Assay—SDMA In-Cell Western Protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 10% CO2 tissue culture incubator. The SAM-cooperative PRMT5 inhibitor, GSK3326595, was sourced from SelleckChem and maintained as a 10 mM DMSO stock. All test compounds are maintained as 10 mM DMSO stocks.

On Day 0, MTAP-intact or MTAP-deleted cells are seeded in a 384-well plate, and incubated in a humidified, 5% CO2 tissue culture incubator for 16-24 hours. On Day 1, the test compounds are dispensed to wells at defined concentrations using a Tecan D300e digital dispenser (n=4), and the volume of DMSO is normalized to highest class volume. Each plate includes wells dosed with defined concentrations of GSK33226595 as a plate control. The compounds are incubated with cells for 24 hours in a humidified, 5% CO2 tissue culture incubator.

On Day 2, the compound-treated cells are fixed with a final concentration of 4% formaldehyde. The cells are then washed/permeabilized with 1×PBS+0.1% Triton X-100, and then blocked with 5% goat serum/1×TBS. The fixed cells are then incubated overnight at 4° C. with a primary SDMA antibody cocktail (Cell Signaling 13222).

On Day 3, the cells are washed with 1×PBS+0.1% Triton X-100, and then incubated at room temperature for 1 hour with a NIR fluorescent secondary antibody cocktail that also contains DRAQ5 (LiCor 926-32211 and VWR 10761-508). The cells are washed with 1×PBS+0.1% Triton X-100, and then washed again with ddH2O. The plates are then imaged using a NIR fluorescent imager (LiCor Odyssey).

For data analysis, the SDMA signal is normalized to the DRAQ5 signal. Assay background is determined by the signal from wells treated with 1 μM GSK3326595, and subtracted from every well. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC50 fitting procedure in Dotmatics Studies 5.4 as part of a customized data analysis protocol.

The data obtained in this experiment is presented in Table 1, columns 4-6.

### Example 247. Viability Assay Protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 5 or 10% CO2 tissue culture incubator. All test compounds are maintained as 10 mM DMSO stocks.

On Day 0, MTAP-intact and MTAP-deleted cells are seeded in a 96-well plate, and incubated in a humidified, 5 or 10% CO2 tissue culture incubator for 16-24 hours. On Day 1, the test compounds are dispensed to wells at defined concentrations using a Tecan D300e digital dispenser (n=3), and the volume of DMSO is normalized to highest class volume (0.2%). The compound-treated plates are incubated for 7 days in a humidified, 5 or 10% CO2 tissue culture incubator.

On Day 7, the plates are removed from the tissue culture incubator and allowed to equilibrate to room temperature. Then either a ½ volume CellTiter-Glo Luminescent Cell Viability Assay reagent (Promega G7572) is added to each well, or the media is removed from every well and a 1:3 dilution of CellTiter-Glo 2.0 Cell Viability Assay reagent (Promega G9241) in 1×PBS is added. Ten minutes after addition, the luminescent signal is detected by an Envision plate reader. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC50 fitting procedure in Dotmatics Studies 5.4 as part of a customized data analysis protocol.

The data obtained in this experiment is presented in Table 1, column 8.

### Example 248. Combination Viability Assay Protocol

A SW1573 MTAP-isogenic cell line pair can be generated by either reconstituting MTAP gene expression, or by introducing an empty control vector, in the MTAP-deleted SW1573 parental cell line. The cell lines can be maintained in DMEM+10% FBS in a humidified, 5% CO2 tissue culture incubator. All test compounds can be maintained as 10 mM DMSO stocks.

On Day 0, MTAP-intact and MTAP-deleted cells can be seeded in a 384-well plate, and incubated in a humidified, 5% CO2 tissue culture incubator for 16-24 hours. On Day 1, the test compounds can be dispensed to wells at defined concentrations (n=2), and the volume of DMSO can be normalized to highest class volume. The compound-treated plates can be incubated for 7 days in a humidified, 5% CO2 tissue culture incubator.

On Day 7, the plates can be removed from the tissue culture incubator and allowed to equilibrate to room temperature. Relative viability can be assessed by addition of CellTiter-Glo reagent, and data can be plotted as % of DMSO control for each compound in each cell line, with a 4-parameter fit non-linear regression model (GraphPad Prism). Synergy can be determined according to the HSA model by the Combenefit software package (Version 2.021).

### PRMT5 Inhibitors and MAT2A Inhibitors Represents a Potential Clinical Combination in MTAP-Deleted Tumors

Marjon et al (Cell Reports 2016) and Kalev et al (Cancer Cell 2021) identify MAT2A as a therapeutic target in MTAP-deleted tumors. The combination of a MAT2A inhibitor with an inhibitor that selectively targets PRMT5 in MTAP-null cells can be assessed to determine whether this would present a rational therapeutic strategy. Combination of a MAT2A inhibitor (e.g., AG-270) with an exemplar MTAP^{null}-selective PRMT5 inhibitor in a 7-day viability assay in the MTAP-null SW1573 cancer cell line can demonstrate enhanced cellular viability defects.

### PRMT5 Inhibitors and MAPK or KRASG12C Inhibitors Represent a Potential Clinical Combination in MTAP-Deleted, KRAS-Mutated Tumors

MTAP-deletion can co-occur with mutations in the KRAS gene (e.g., KRASG12C). Therapies targeting KRAS or other members of the MAPK pathway (eg, MAPK3, MAPK1, MEK1 and MEK2) exist. The combination of these inhibitors with an inhibitor that selectively targets PRMT5 in MTAP-null cells can be assessed to determine whether this would present a therapeutic strategy.

Combination of a KRASG12C inhibitor (e.g., AMG-510), with an exemplar MTAP^{null}-selective PRMT5 inhibitor in a 7-day viability assay in the MTAP-null SW1573 cancer cell line can demonstrate enhanced cellular viability defects.

Combination of MAPK1/MAPK3 inhibitors (e.g., ulixertinib and SCH772984), with an exemplar MTAP^{null}-selective PRMT5 inhibitor in a 7-day viability assay in the MTAP-null SW1573 cancer cell line can demonstrate enhanced cellular viability defects.

Combination of MEK inhibitors (e.g., trametinib) with an exemplar MTAP^{null}-selective PRMT5 inhibitor in a 7-day viability assay in the MTAP-null SW1573 cancer cell line can demonstrate enhanced cellular viability defects.

### Selected Embodiments

Embodiment 1. A compound of Formula (A) or Formula (I) or a pharmaceutically acceptable salt thereof, wherein:

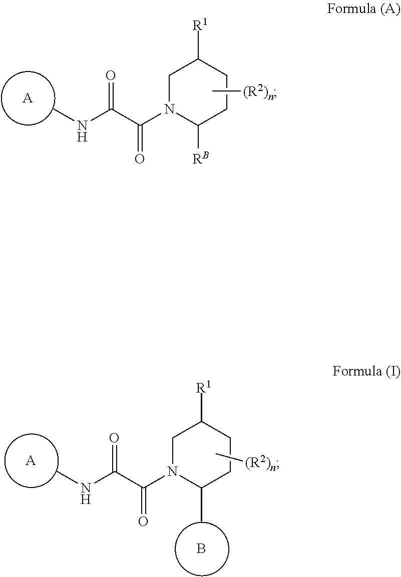

Ring A is an optionally substituted fused bicyclic 8-10 membered heteroaryl ring system containing at least one nitrogen atom, wherein the 8-10 membered refers to the total number of atoms in the fused system;
R^{B} is an optionally substituted C₁-C₆ alkyl;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
   provided that:
(i) when the compound is of Formula (A), Ring A is a 3-pyridine fused with a 5-membered heteroaryl via 2 carbon atoms; and
(ii) the compound is not one of compounds a. to d. or a pharmaceutically acceptable salt thereof:
a. N-(8-fluoroquinolin-3-yl)-2-oxo-2-(2-phenylpiperidin-1-yl)acetamide

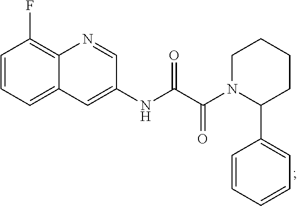 [SMILES: O=C(Nc1cnc2c(F)cccc2c1)C(=O)N1CCCCC1c1ccccc1]

b. 2-(2-(1H-pyrazol-4-yl)piperidin-1-yl)-N-(1H-indol-4-yl)-2-oxoacetamide

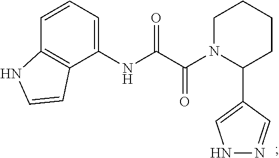 [SMILES: O=C(Nc1cccc2[nH]ccc12)C(=O)N1CCCCC1c1cn[nH]c1]

c. 2-(2-(4-bromophenyl)piperidin-1-yl)-2-oxo-N-(pyrazolo[1,5-a]pyrimidin-3-yl)acetamide

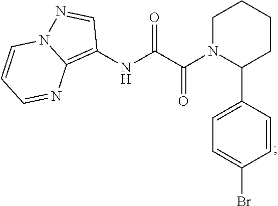 [SMILES: O=C(Nc1cnn2cccnc12)C(=O)N1CCCCC1c1ccc(Br)cc1]

d. 2-(2-(1H-pyrazol-4-yl)piperidin-1-yl)-N-(1H-indazol-5-yl)-2-oxoacetamide

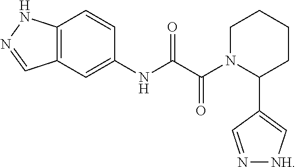 [SMILES: O=C(Nc1ccc2[nH]ncc2c1)C(=O)N1CCCCC1c1cn[nH]c1]

Embodiment 2. The compound of embodiment 1, or a pharmaceutically acceptable salt thereof, wherein the compound is of Formula (A).
Embodiment 3. The compound of embodiment 1 or 2, or a pharmaceutically acceptable salt thereof, wherein R^{B} is —C₁-C₆ alkyl substituted with 0, 1, 2 or 3 substituents independently selected from ═O, —F, —Cl, —OH—, —OMe, —NH₂ and —CN.
Embodiment 4. The compound of embodiment 1 or 2, or a pharmaceutically acceptable salt thereof, wherein R^{B} is unsubstituted-C₁-C₆ alkyl.
Embodiment 5. The compound of embodiment 1 or 2, or a pharmaceutically acceptable salt thereof, wherein R^{B} is selected from -Me, -Et, -Pr, -ⁱPr and -^{t}Bu.
Embodiment 6. The compound of embodiment 1 or 2, or a pharmaceutically acceptable salt thereof, wherein R^{B} is selected from -Me, -Et, -Pr and -^{t}Bu.
Embodiment 7. The compound of embodiment 1, or a pharmaceutically acceptable salt thereof, wherein the compound is of Formula (I):

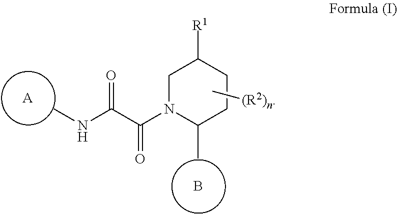

Embodiment 8. A compound of Formula (I):

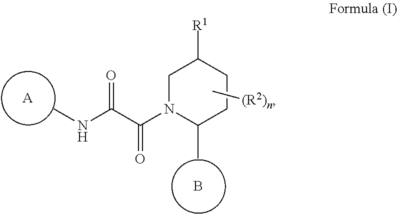

or a pharmaceutically acceptable salt thereof,
wherein:
Ring A is an optionally substituted fused bicyclic 8-10 membered heteroaryl ring system containing at least one nitrogen atom, wherein the 8-10 membered refers to the total number of atoms in the fused system;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O) Ra, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Structure I can form a bridged, fused or spiro bicyclic heterocyclic ring);
each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, -C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
provided that the compound is not one of compounds a. to d. or a pharmaceutically acceptable salt thereof:
a. N-(8-fluoroquinolin-3-yl)-2-oxo-2-(2-phenylpiperidin-1-yl)acetamide

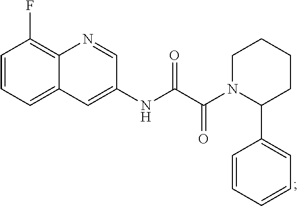 [SMILES: O=C(Nc1cnc2c(F)cccc2c1)C(=O)N1CCCCC1c1ccccc1]

b. 2-(2-(1H-pyrazol-4-yl)piperidin-1-yl)-N-(1H-indol-4-yl)-2-oxoacetamide

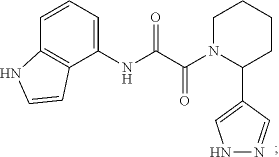 [SMILES: O=C(Nc1cccc2[nH]ccc12)C(=O)N1CCCCC1c1cn[nH]c1]

c. 2-(2-(4-bromophenyl)piperidin-1-yl)-2-oxo-N-(pyrazolo[1,5-a]pyrimidin-3-yl)acetamide

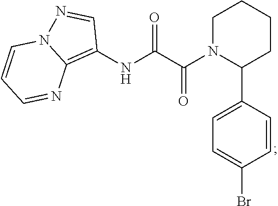 [SMILES: O=C(Nc1cnn2cccnc12)C(=O)N1CCCCC1c1ccc(Br)cc1]

d. 2-(2-(1H-pyrazol-4-yl)piperidin-1-yl)-N-(1H-indazol-5-yl)-2-oxoacetamide

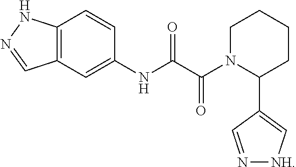 [SMILES: O=C(Nc1ccc2[nH]ncc2c1)C(=O)N1CCCCC1c1cn[nH]c1]

Embodiment 9. A compound of Formula (I):

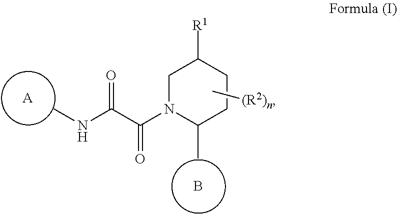

or a pharmaceutically acceptable salt thereof,
wherein:
Ring A is an optionally substituted fused bicyclic 8-10 membered heteroaryl ring system containing at least one nitrogen atom, wherein the 8-10 membered refers to the total number of atoms in the fused system; Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Structure I can form a bridged, fused or spiro bicyclic heterocyclic ring);
each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
   provided that the compound is not:
a. N-(8-fluoroquinolin-3-yl)-2-oxo-2-(2-phenylpiperidin-1-yl)acetamide;
b. 2-(2-(1H-pyrazol-4-yl)piperidin-1-yl)-N-(1H-indol-4-yl)-2-oxoacetamide;
c. 2-(4-methyl-2-(5-methylfuran-2-yl)piperidin-1-yl)-2-oxo-N-(4,5,6,7-tetrahydro-1H-indazol-7-yl)acetamide;
d. 2-(2-(1H-pyrazol-4-yl)piperidin-1-yl)-N-(1H-indazol-5-yl)-2-oxoacetamide.
Embodiment 10. The compound of any one of embodiments 1-6, or a pharmaceutically acceptable salt thereof, wherein R¹ is not H and R^{B} and R¹ are in a trans relative configuration.
Embodiment 11. The compound of any one of embodiments 1-6, or a pharmaceutically acceptable salt thereof, wherein R¹ is not H and R^{B} and R¹ are in a cis relative configuration.
Embodiment 12. The compound of any one of embodiments 1 and 7-9, or a pharmaceutically acceptable salt thereof, wherein R¹ is not H and Ring B and R¹ are in a trans relative configuration.
Embodiment 13. The compound of any one of embodiments 1 and 7-9, or a pharmaceutically acceptable salt thereof, wherein R¹ is not H and Ring B and R¹ are in a cis relative configuration.
Embodiment 14. The compound of any one of embodiments 1 and 7-9, or a pharmaceutically acceptable salt thereof, wherein the moiety represented as is

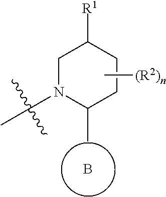

selected from the group consisting of:

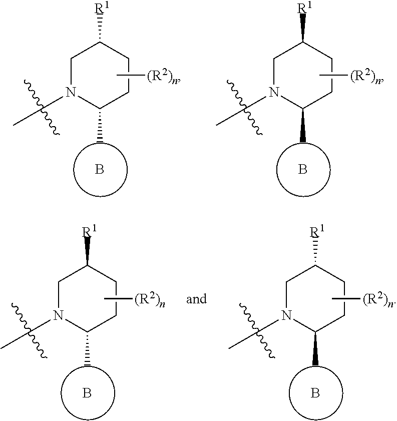

Embodiment 15. The compound of any one of embodiments 1 and 7-9, or a pharmaceutically acceptable salt thereof, wherein the moiety represented as

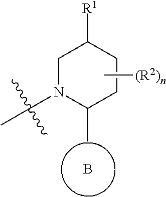

is selected from the group consisting of:

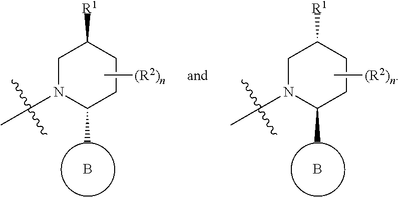

Embodiment 16. The compound of any one of embodiments 1 and 7-9, or a pharmaceutically acceptable salt thereof, wherein the moiety represented as

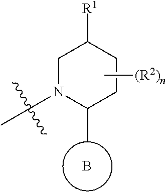

is selected from the group consisting of:

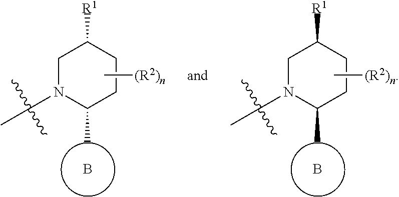

Embodiment 17. The compound of embodiment 15, or a pharmaceutically acceptable salt thereof, wherein the compound is of Formula (Ia):

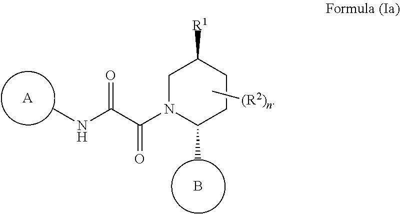

Embodiment 18. The compound of embodiment 15, or a pharmaceutically acceptable salt thereof, wherein the compound is of Formula (Ib):

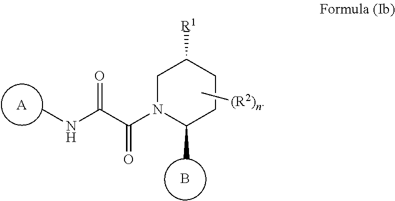

Embodiment 19. The compound of embodiment 16, or a pharmaceutically acceptable salt thereof, wherein the compound is of Formula (Ic):

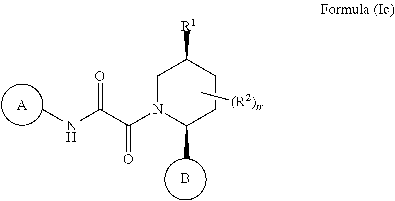

Embodiment 20. The compound of embodiment 16, or a pharmaceutically acceptable salt thereof, wherein the compound is of Formula (Id):

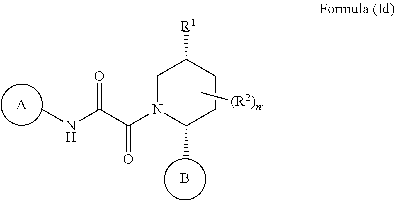

Embodiment 21. The compound of any one of embodiments 1 to 20, or a pharmaceutically acceptable salt thereof, wherein Ring A is a fused bicyclic 8-10 membered heteroaryl ring containing at least one nitrogen atom, substituted at any available positions with 0, 1, 2 or 3 instances of R⁴, wherein the 8-10 membered refers to the total number of atoms in the fused system;
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂; and
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl).
Embodiment 22. The compound of any one of embodiments 1 to 20, or a pharmaceutically acceptable salt thereof, wherein Ring A is selected from the group consisting of:

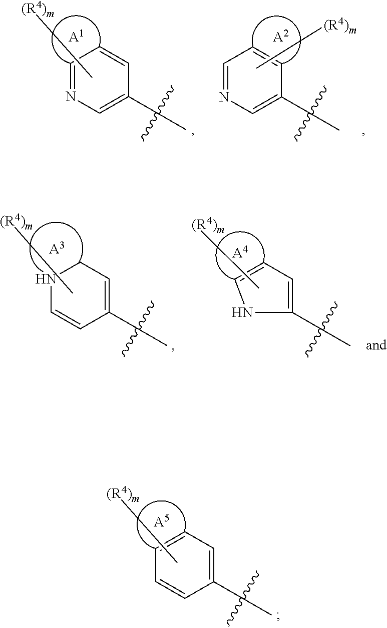

wherein
each of rings A¹, A² and A⁴ is independently 4-6 membered carbocyclyl, 4-6 membered heterocyclyl, 5-6 membered heteroaryl or phenyl;
each ring A³ is independently a 4-6 membered heterocyclyl or 5-6 membered heteroaryl, wherein the heterocyclyl and heteroaryl contain at least one nitrogen atom each ring A⁵ is independently a 5-6 membered heteroaryl, wherein the heteroaryl contains at least one nitrogen atom;
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
m is 0, 1, 2 or 3.
Embodiment 23. The compound of embodiment 21, or a pharmaceutically acceptable salt thereof, wherein Ring A is selected from the group consisting of:

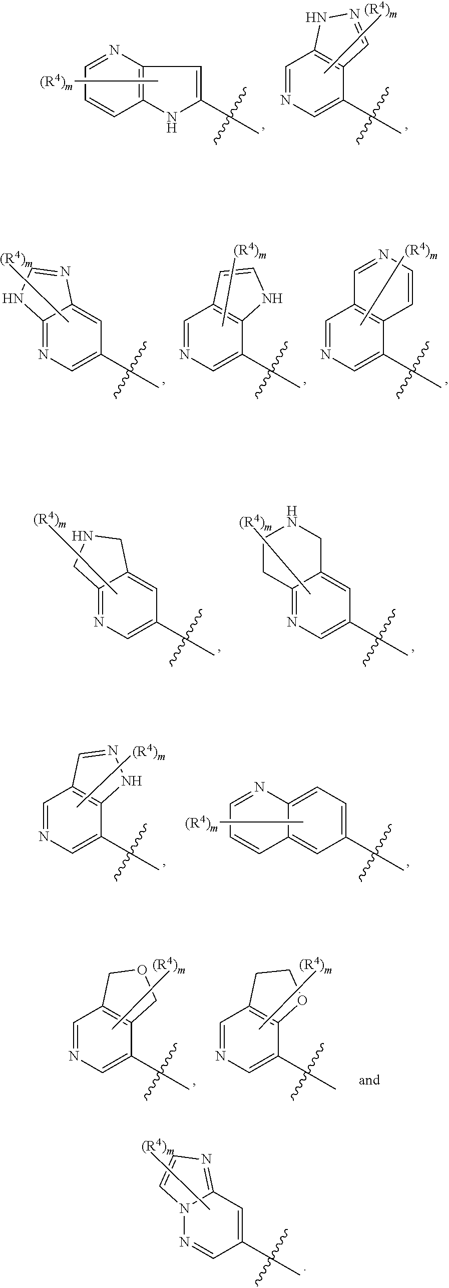

Embodiment 24. The compound of embodiment 21, or a pharmaceutically acceptable salt thereof, wherein Ring A is selected from the group consisting of:

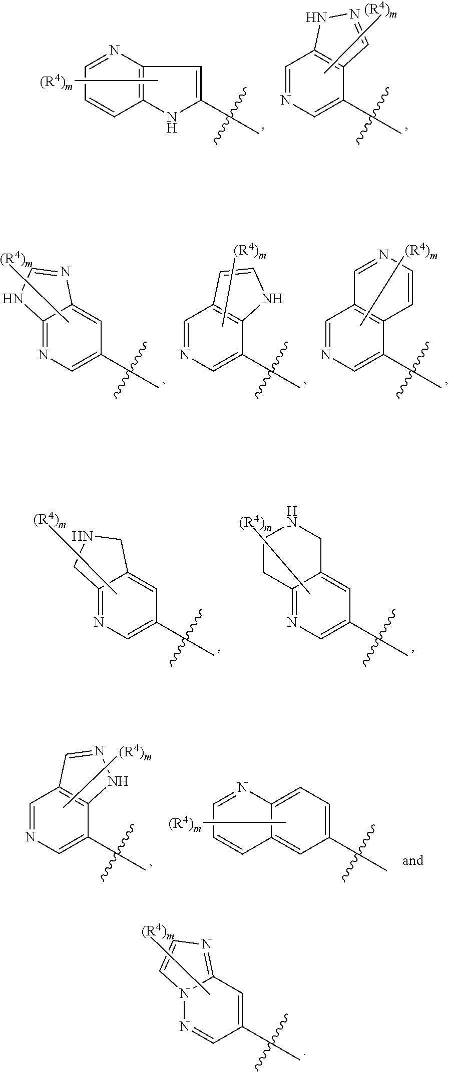

Embodiment 25. The compound of embodiment 21, or a pharmaceutically acceptable salt thereof, wherein Ring A is selected from the group consisting of:

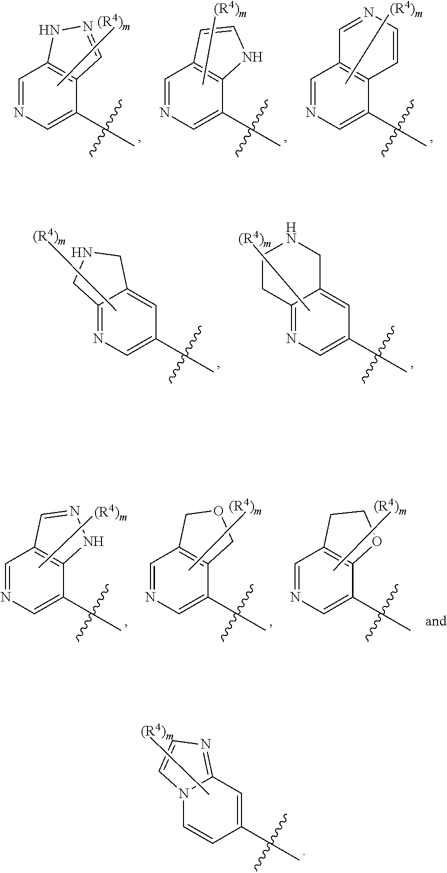

Embodiment 26. The compound of embodiment 21, or a pharmaceutically acceptable salt thereof, wherein Ring A is

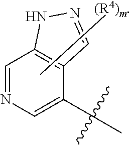

Embodiment 27. The compound of embodiment 21, or a pharmaceutically acceptable salt thereof, wherein Ring A is

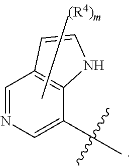

Embodiment 28. The compound of embodiment 21, or a pharmaceutically acceptable salt thereof, wherein Ring A is

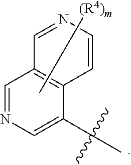

Embodiment 29. The compound of embodiment 21, or a pharmaceutically acceptable salt thereof, wherein Ring A is

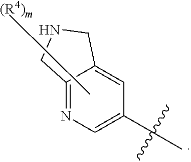

Embodiment 30. The compound of embodiment 21, or a pharmaceutically acceptable salt thereof, wherein Ring A is

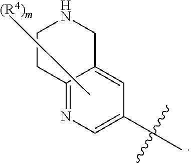

Embodiment 31. The compound of embodiment 21, or a pharmaceutically acceptable salt thereof, wherein Ring A is

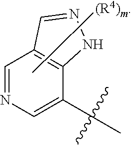

Embodiment 32. The compound of embodiment 21, or a pharmaceutically acceptable salt thereof, wherein Ring A is

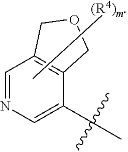

Embodiment 33. The compound of embodiment 21, or a pharmaceutically acceptable salt thereof, wherein Ring A is

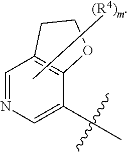

Embodiment 34. The compound of embodiment 21, or a pharmaceutically acceptable salt thereof, wherein Ring A is

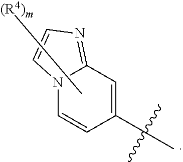

Embodiment 35. The compound of any one of embodiments 21 to 34, or a pharmaceutically acceptable salt thereof, wherein m is 0 or 1.
Embodiment 36. The compound of embodiment 35, or a pharmaceutically acceptable salt thereof, wherein m is 0.
Embodiment 37. The compound of embodiment 35, or a pharmaceutically acceptable salt thereof, wherein m is 1.
Embodiment 38. The compound of any one of embodiments 21 to 34, or a pharmaceutically acceptable salt thereof, wherein m is 2.
Embodiment 39. The compound of any one of embodiments 21 to 34, or a pharmaceutically acceptable salt thereof, wherein m is 3.
Embodiment 40. The compound of embodiment 21, or a pharmaceutically acceptable salt thereof, wherein Ring A is selected from the group consisting of:

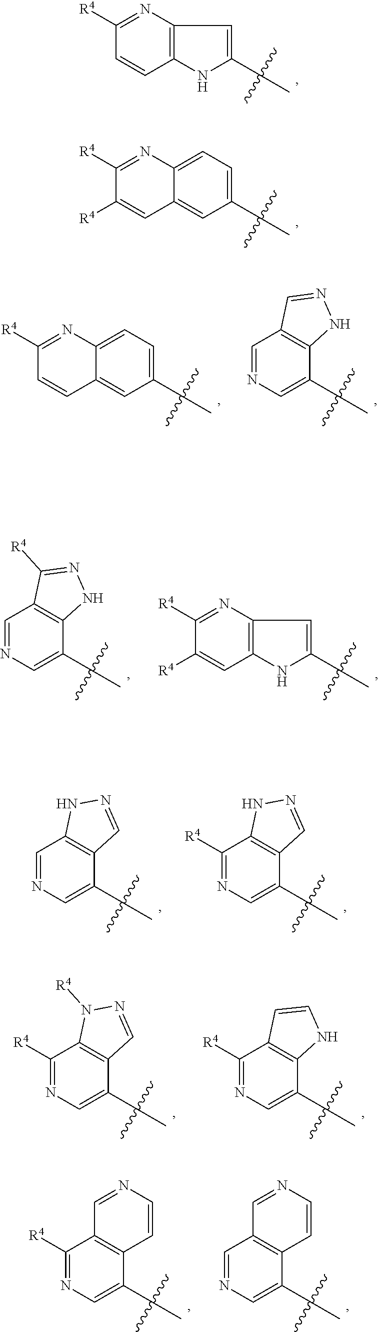

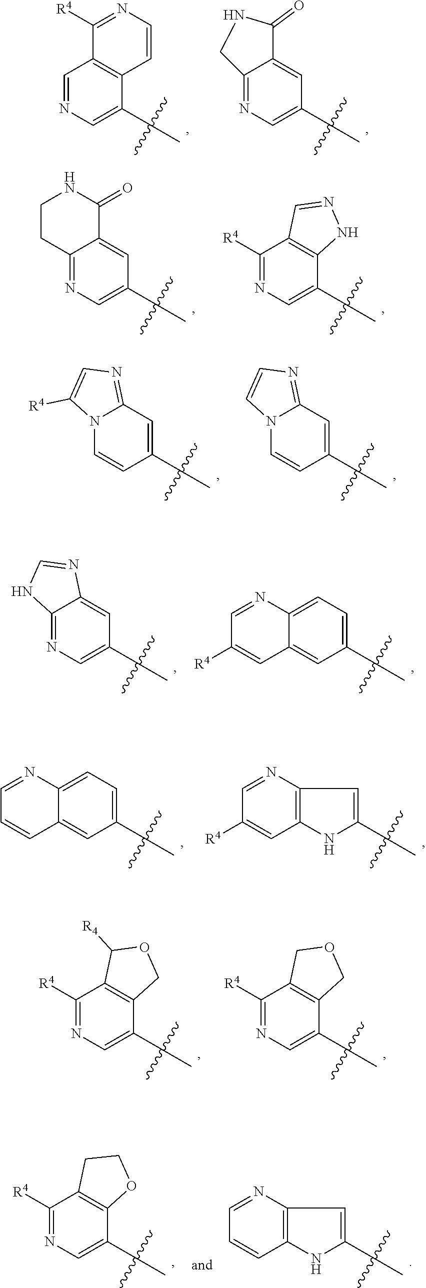

Embodiment 41. The compound of embodiment 21, or a pharmaceutically acceptable salt thereof, wherein Ring A is selected from the group consisting of:

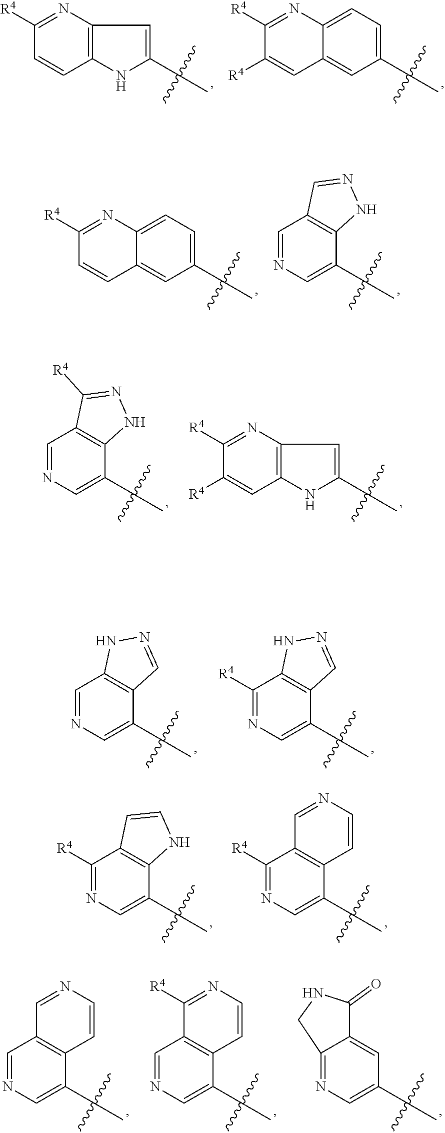

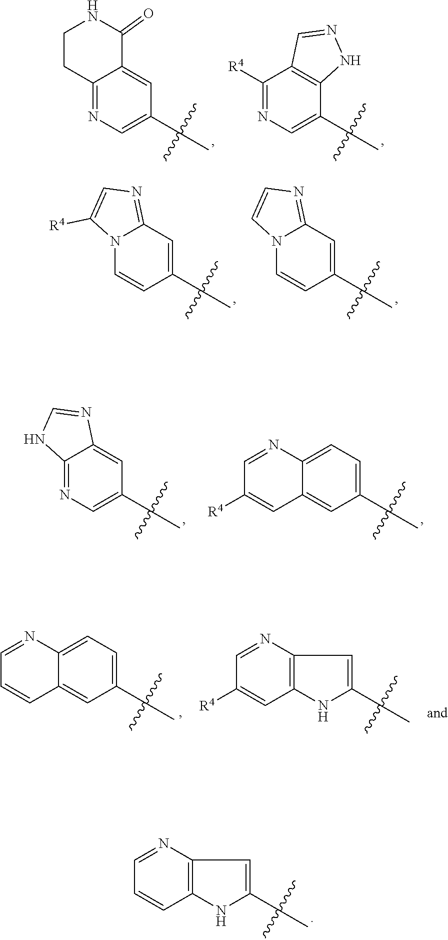

Embodiment 42. The compound of embodiment 21, or a pharmaceutically acceptable salt thereof, wherein Ring A is selected from the group consisting of:

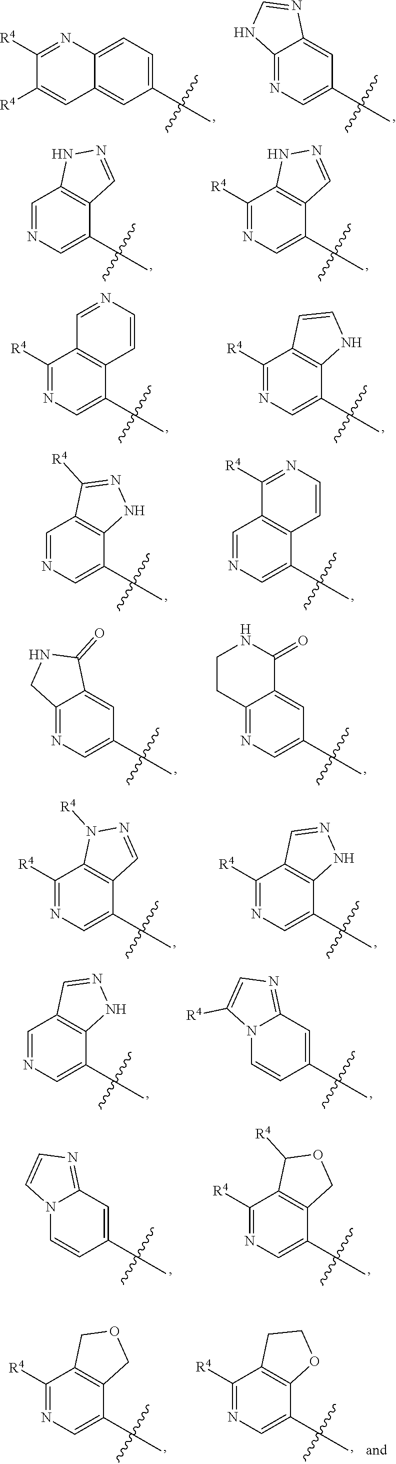

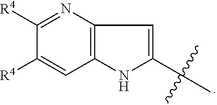

Embodiment 43. The compound of embodiment 21, or a pharmaceutically acceptable salt thereof, wherein Ring A is selected from the group consisting of:

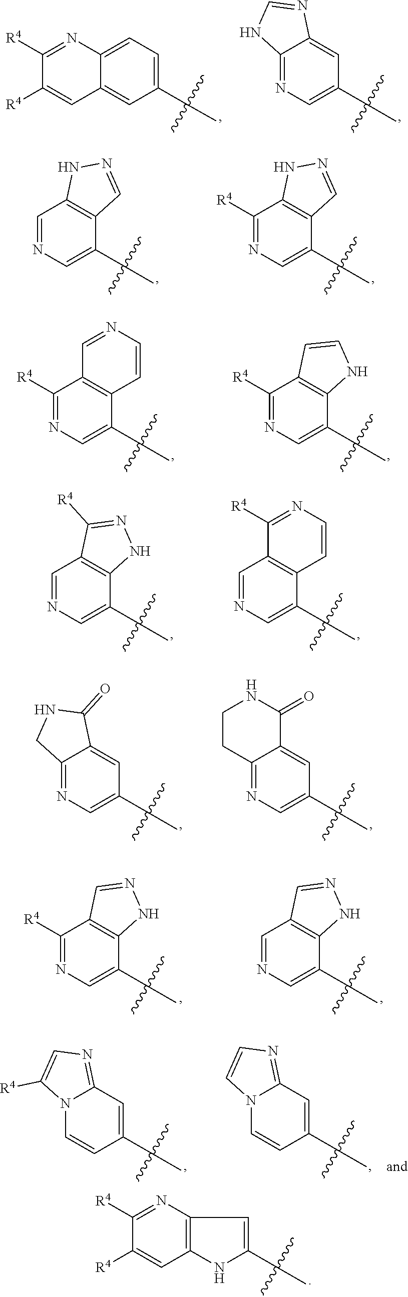

Embodiment 44. The compound of embodiment 21, or a pharmaceutically acceptable salt thereof, wherein Ring A is selected from the group consisting of:

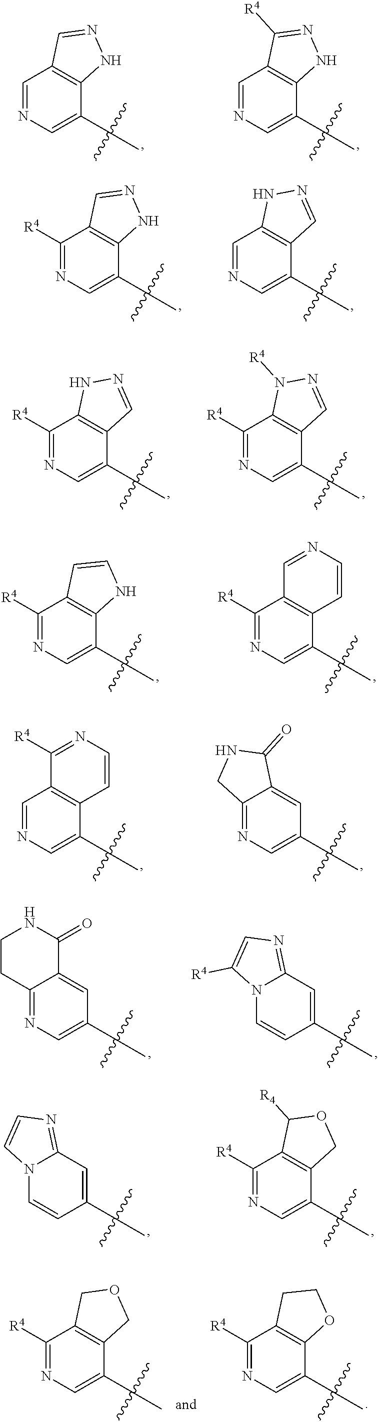

Embodiment 45. The compound of embodiment 21, or a pharmaceutically acceptable salt thereof, wherein Ring A is

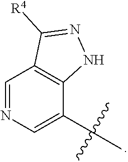

Embodiment 46. The compound of embodiment 21, or a pharmaceutically acceptable salt thereof, wherein Ring A is

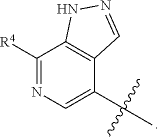

Embodiment 47. The compound of embodiment 21, or a pharmaceutically acceptable salt thereof, wherein Ring A is

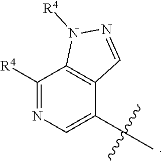

Embodiment 48. The compound of embodiment 21, or a pharmaceutically acceptable salt thereof, wherein Ring A is

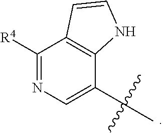

Embodiment 49. The compound of embodiment 21, or a pharmaceutically acceptable salt thereof, wherein Ring A is

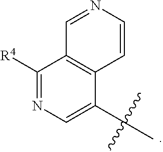

Embodiment 50. The compound of embodiment 21, or a pharmaceutically acceptable salt thereof, wherein Ring A is

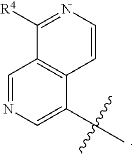

Embodiment 51. The compound of embodiment 21, or a pharmaceutically acceptable salt thereof, wherein Ring A is

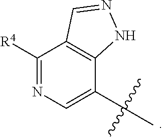

Embodiment 52. The compound of embodiment 21, or a pharmaceutically acceptable salt thereof, wherein Ring A is

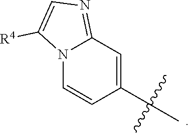

Embodiment 53. The compound of embodiment 21, or a pharmaceutically acceptable salt thereof, wherein Ring A is

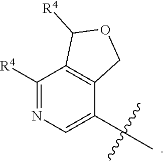

Embodiment 54. The compound of embodiment 21, or a pharmaceutically acceptable salt thereof, wherein Ring A is

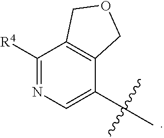

Embodiment 55. The compound of embodiment 21, or a pharmaceutically acceptable salt thereof, wherein Ring A is

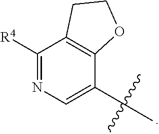

Embodiment 56. The compound of embodiment 21, or a pharmaceutically acceptable salt thereof, wherein Ring A is

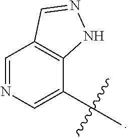 [SMILES: *c1cncc2cn[nH]c12]

Embodiment 57. The compound of embodiment 21, or a pharmaceutically acceptable salt thereof, wherein Ring A is

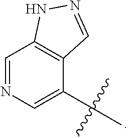 [SMILES: *c1cncc2[nH]ncc12]

Embodiment 58. The compound of embodiment 21, or a pharmaceutically acceptable salt thereof, wherein Ring A is

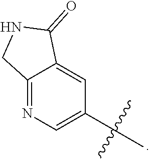 [SMILES: *c1cnc2c(c1)C(=O)NC2]

Embodiment 59. The compound of embodiment 21, or a pharmaceutically acceptable salt thereof, wherein Ring A is

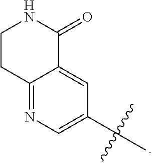 [SMILES: *c1cnc2c(c1)C(=O)NCC2]

Embodiment 60. The compound of embodiment 21, or a pharmaceutically acceptable salt thereof, wherein Ring A is

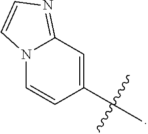 [SMILES: *c1ccn2ccnc2c1]

Embodiment 61. The compound of any one of embodiments 21 to 55, or a pharmaceutically acceptable salt thereof, wherein each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂.
Embodiment 62. The compound of any one of embodiments 21 to 55, or a pharmaceutically acceptable salt thereof, wherein each R⁴ is independently selected from the group consisting of -D, —O, —C₁-C₆ alkyl, —C₁-C₆ haloalkyl, —C₃-C₉ cycloalkyl, —OR^{a4} and —N(R^{a4})₂.
Embodiment 63. The compound of any one of embodiments 21 to 55, or a pharmaceutically acceptable salt thereof, wherein each R⁴ is independently selected from the group consisting of -D, ═O, —C₁-C₆ alkyl and —N(R^{a4})₂.
Embodiment 64. The compound of any one of embodiments 21 to 55 and 61 to 63, or a pharmaceutically acceptable salt thereof, wherein each R^{a4} is independently selected from the group consisting of H and C₁-C₆ alkyl.
Embodiment 65. The compound of any one of embodiments 21 to 55 and 61 to 63, or a pharmaceutically acceptable salt thereof, wherein each R^{a4} is H.
Embodiment 66. The compound of any one of embodiments 21 to 55, or a pharmaceutically acceptable salt thereof, wherein each R⁴ is independently selected from the group consisting of -D, ═O, -Me, -Et, -Pr, -^{t}Bu, —NH₂, —NHCH₃ and —NH(CH₃)₂.
Embodiment 67. The compound of any one of embodiments 21 to 55, or a pharmaceutically acceptable salt thereof, wherein each R⁴ is independently —NHCH₃, —NH₂ or -Me.
Embodiment 68. The compound of any one of embodiments 21 to 55, or a pharmaceutically acceptable salt thereof, wherein each R⁴ is independently —NH₂ or -Me.
Embodiment 69. The compound of any one of embodiments 21 to 55, or a pharmaceutically acceptable salt thereof, wherein each R⁴ is —NH₂.
Embodiment 70. The compound of any one of embodiments 21 to 55, or a pharmaceutically acceptable salt thereof, wherein each R⁴ is -Me.
Embodiment 71. The compound of any one of embodiments 1 to 70, or a pharmaceutically acceptable salt thereof, wherein Ring A is selected from the group consisting of:

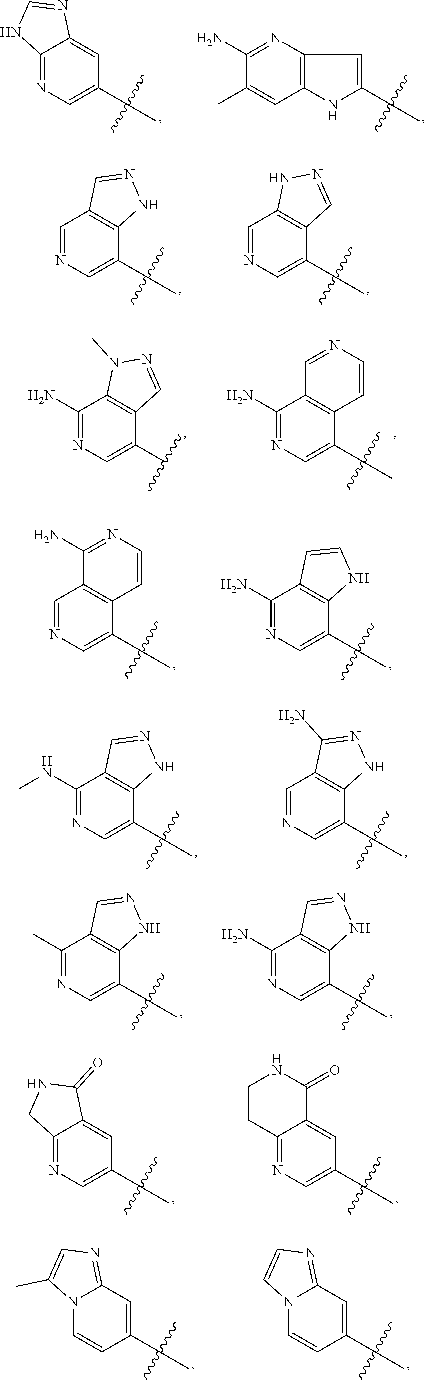

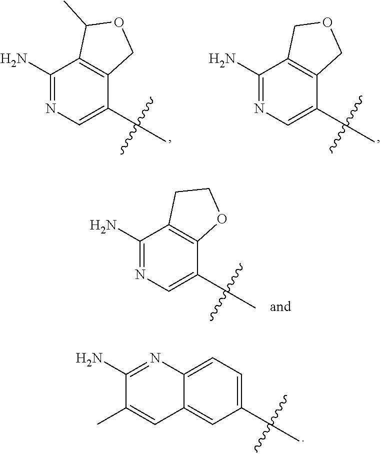 [SMILES: *c1cnc(N)c2c1COC2C ; *c1cnc(N)c2c1COC2 ; *c1cnc(N)c2c1OCC2 ; *c1ccc2nc(N)c(C)cc2c1]

Embodiment 72. The compound of any one of embodiments 1 to 70, or a pharmaceutically acceptable salt thereof, wherein Ring A is selected from the group consisting of:

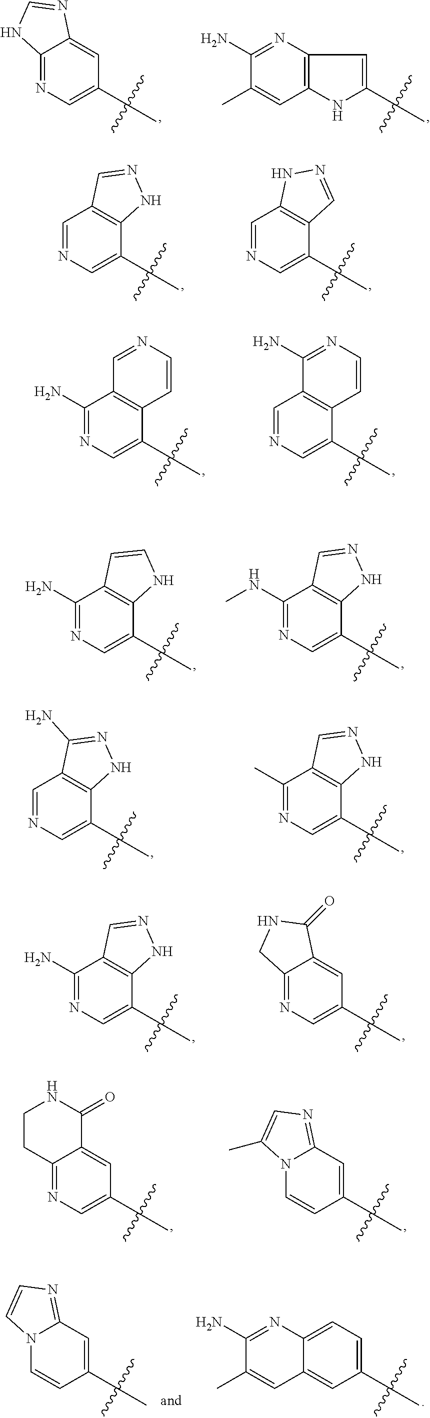

Embodiment 73. The compound of any one of embodiments 1 to 70, or a pharmaceutically acceptable salt thereof, wherein Ring A is selected from the group consisting of:

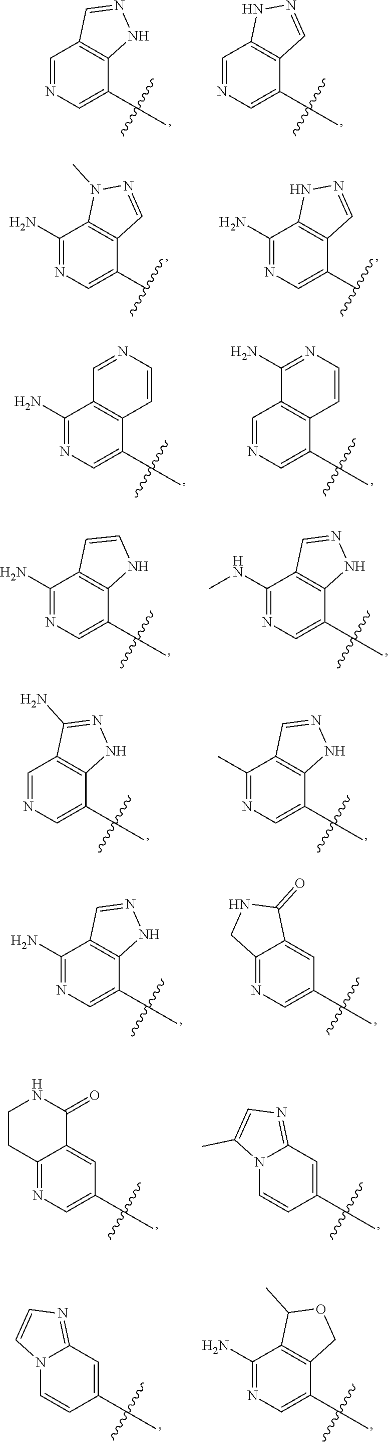

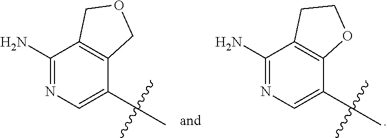 [SMILES: *c1cnc(N)c2c1COC2 ; *c1cnc(N)c2c1OCC2]

Embodiment 74. The compound of embodiment 21, or a pharmaceutically acceptable salt thereof, wherein Ring A is

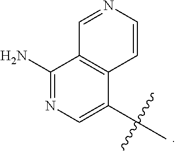 [SMILES: *c1cnc(N)c2cnccc12]

Embodiment 75. The compound of embodiment 21, or a pharmaceutically acceptable salt thereof, wherein Ring A is

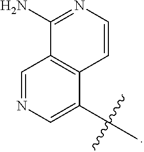 [SMILES: *c1cncc2c(N)nccc12]

Embodiment 76. The compound of embodiment 21, or a pharmaceutically acceptable salt thereof, wherein Ring A is

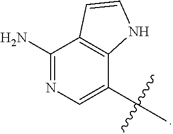 [SMILES: *c1cnc(N)c2cc[nH]c12]

Embodiment 77. The compound of embodiment 21, or a pharmaceutically acceptable salt thereof, wherein Ring A is

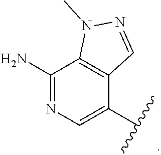 [SMILES: *c1cnc(N)c2c1cnn2C]

Embodiment 78. The compound of embodiment 21, or a pharmaceutically acceptable salt thereof, wherein Ring A is

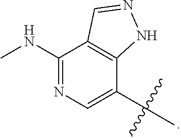 [SMILES: *c1cnc(NC)c2cn[nH]c12]

Embodiment 79. The compound of embodiment 21, or a pharmaceutically acceptable salt thereof, wherein Ring A is

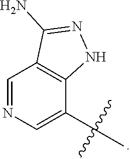 [SMILES: *c1cncc2c(N)n[nH]c12]

Embodiment 80. The compound of embodiment 21, or a pharmaceutically acceptable salt thereof, wherein Ring A is

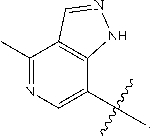 [SMILES: *c1cnc(C)c2cn[nH]c12]

Embodiment 81. The compound of embodiment 21, or a pharmaceutically acceptable salt thereof, wherein Ring A is

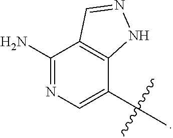 [SMILES: *c1cnc(N)c2cn[nH]c12]

Embodiment 82. The compound of embodiment 21, or a pharmaceutically acceptable salt thereof, wherein Ring A is

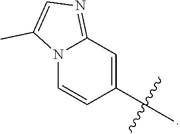 [SMILES: *c1ccn2c(C)cnc2c1]

Embodiment 83. The compound of embodiment 21, or a pharmaceutically acceptable salt thereof, wherein Ring A is

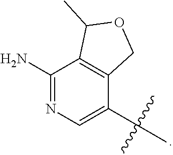 [SMILES: *c1cnc(N)c2c1COC2C]

Embodiment 84. The compound of embodiment 21, or a pharmaceutically acceptable salt thereof, wherein Ring A is

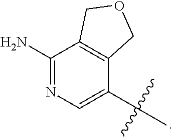 [SMILES: *c1cnc(N)c2c1COC2]

Embodiment 85. The compound of embodiment 21, or a pharmaceutically acceptable salt thereof, wherein Ring A is

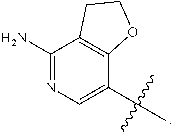 [SMILES: *c1cnc(N)c2c1OCC2]

Embodiment 86. The compound of any one of embodiments 1 to 85, or a pharmaceutically acceptable salt thereof, wherein each R¹ is independently selected from the group consisting of H, halo (e.g., —F, —Cl), —CN, —C₁-C₆ alkyl (e.g., -Me, -Et, -Pr, -ⁱPr, -sec-Bu, —^{t}Bu), 5-membered heteroaryl (e.g., pyrazolyl), —C₁-C₆ haloalkyl (e.g., —CF₃, —CHF₂, —CH₂CF₃), —C₁-C₆ heteroalkyl (e.g., —CH₂OH), —C₃-C₉ cycloalkyl (e.g., cyclopropyl, cyclobutyl, cyclopentyl, cyclohexyl), —OR^{a1} (e.g., —OH, —OCH₃, —OCHF₂), —N(R^{a1})₂ and —C(═O)N(R^{a1})₂ (e.g., —C(═O)NH₂, —C(═O)NHCH₃).
Embodiment 87. The compound of any one of embodiments 1 to 86, or a pharmaceutically acceptable salt thereof, wherein each R^{a1} is independently selected from the group consisting of H and —C₁-C₆ alkyl (e.g., -Me, -Et, -Pr, -ⁱPr, -ⁿBu, -^{t}Bu, -sec-Bu, -iso-Bu).
Embodiment 88. The compound of any one of embodiments 1 to 86, or a pharmaceutically acceptable salt thereof, wherein each R¹ is independently selected from the group consisting of H, -Me and -Et.
Embodiment 89. The compound of any one of embodiments 1 to 86, or a pharmaceutically acceptable salt thereof, wherein each R¹ is independently selected from the group consisting of —H and -Me.
Embodiment 90. The compound of any one of embodiments 1 to 86, or a pharmaceutically acceptable salt thereof, wherein R¹ is -Me.
Embodiment 91. The compound of any one of embodiments 1 to 86, or a pharmaceutically acceptable salt thereof, wherein R¹ is —H.
Embodiment 92. The compound of any one of embodiments 1 to 86, or a pharmaceutically acceptable salt thereof, wherein R¹ is -Et.
Embodiment 93. The compound of any one of embodiments 1, 7-9, 12, 14, 15, 17 and 21-90, or a pharmaceutically acceptable salt thereof, wherein the compound is of Formula (Ia1):

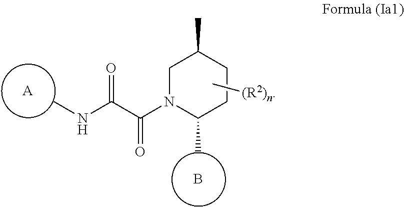

Embodiment 94. The compound of any one of embodiments 1, 7-9, 12, 14, 15, 18 and 21-90, or a pharmaceutically acceptable salt thereof, wherein the compound is of Formula (Ib1):

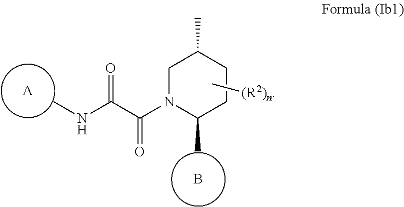

Embodiment 95. The compound of any one of embodiments 1, 7-9, 12, 14, 15, 16, 19 and 21-90, or a pharmaceutically acceptable salt thereof, wherein the compound is of Formula (Ic1):

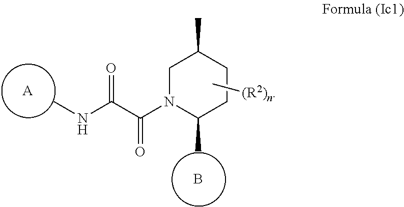

Embodiment 96. The compound of any one of embodiments 1, 7-9, 12, 14, 15, 16 and 20-90, or a pharmaceutically acceptable salt thereof, wherein the compound is of Formula (Id1):

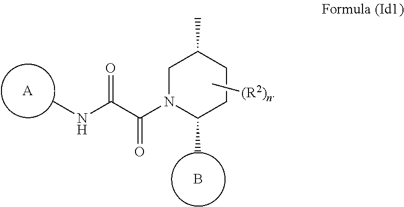

Embodiment 97. The compound of any one of embodiments 1 to 96, or a pharmaceutically acceptable salt thereof, wherein n is 0 or 1.
Embodiment 98. The compound of any one of embodiments 1 to 96, or a pharmaceutically acceptable salt thereof, wherein n is 0.
Embodiment 99. The compound of any one of embodiments 1 to 96, or a pharmaceutically acceptable salt thereof, wherein n is 1.
Embodiment 100. The compound of any one of embodiments 1 to 96, or a pharmaceutically acceptable salt thereof, wherein n is 2.
Embodiment 101. The compound of any one of embodiments 1 to 96, or a pharmaceutically acceptable salt thereof, wherein n is 3.
Embodiment 102. The compound of any one of embodiments 1 to 97 and 99 to 101, or a pharmaceutically acceptable salt thereof, wherein each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂.
Embodiment 103. The compound of any one of embodiments 1 to 97 and 99 to 101, or a pharmaceutically acceptable salt thereof, wherein each R² is independently selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl, cyclobutyl), 3-6 membered heterocyclyl (e.g., oxetanyl, tetrahydrofuranyl), —OR^{a2}, —N(R^{a2})₂, —C(═O)R^{a2}, —C(═O)OR^{a2}, —NR^{a4}C(═O)R^{a2}, —NR^{a2}C(═O)OR^{a2}, —C(═O)N(R^{a2})₂, —OC(═O)N(R^{a2})₂.
Embodiment 104. The compound of any one of embodiments 1 to 97 and 99 to 101, or a pharmaceutically acceptable salt thereof, wherein each R² is independently selected from the group consisting of halo, —CN, —C₁-C₆ alkyl, —C₁-C₆ haloalkyl, —C₃-C₉ cycloalkyl (e.g., cyclopropyl, cyclobutyl), 3-6 membered heterocyclyl (e.g., oxetanyl, tetrahydrofuranyl), —OR^{a2}, —N(R^{a2})₂, —C(═O)R^{a2}, —C(═O)N(OR^{a2}) (R^{a2}), and —C(═O)N(R^{a2})₂.
Embodiment 105. The compound of any one of embodiments 1 to 97 and 99 to 104, or a pharmaceutically acceptable salt thereof, wherein each R^{a2} is independently selected from the group consisting of H and —C₁-C₆ alkyl (e.g., -Me, -Et, -Pr, -ⁱPr, -ⁿBu, -^{t}Bu, -sec-Bu, -iso-Bu).
Embodiment 106. The compound of any one of embodiments 1 to 97 and 99 to 101, or a pharmaceutically acceptable salt thereof, wherein each R² is independently selected from the group consisting of halo (e.g., —Cl), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂), —C₃-C₉ cycloalkyl (e.g., cyclopropyl, cyclobutyl), —C₁-C₆ haloalkoxy, (e.g., —OCF₃, —OCHF₂), —OCH₃, —C(═O) H, —C(═O)NHOH, and —C(═O)NH₂.
Embodiment 107. The compound of any one of embodiments 1 to 97 and 99 to 101, or a pharmaceutically acceptable salt thereof, wherein each R² is independently selected from the group consisting of halo (e.g., —Cl), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂), —C₃-C₉ cycloalkyl (e.g., cyclopropyl, cyclobutyl), and —OCH₃.
Embodiment 108. The compound of any one of embodiments 1 to 97 and 99 to 101, or a pharmaceutically acceptable salt thereof, wherein each R² is independently selected from the group consisting of halo (e.g., —Cl), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂) and —OCH₃.
Embodiment 109. The compound of any one of embodiments 1 to 97 and 99 to 101, or a pharmaceutically acceptable salt thereof, wherein each R² is independently selected from the group consisting of halo (e.g., —Cl), —C₁-C₆ alkyl (e.g., -Me, -Et, -Pr, -ⁱPr, -ⁿBu, -^{t}Bu, -sec-Bu, -iso-Bu) and —OCH₃.
Embodiment 110. The compound of any one of embodiments 1 to 97 and 99 to 101, or a pharmaceutically acceptable salt thereof, wherein each R² is independently selected from the group consisting of —F, -Me, —CF₃ and —OCH₃.
Embodiment 111. The compound of any one of embodiments 1 to 97 and 99 to 101, or a pharmaceutically acceptable salt thereof, wherein R² is selected from the group consisting of -Me, -Et, -^{t}Bu, —CF₃, cyclobutyl and —OCH₃.
Embodiment 112. The compound of any one of embodiments 1 to 97 and 99 to 101, or a pharmaceutically acceptable salt thereof, wherein R² is —F.
Embodiment 113. The compound of any one of embodiments 1 to 97 and 99 to 101, or a pharmaceutically acceptable salt thereof, wherein R² is -Me.
Embodiment 114. The compound of any one of embodiments 1 to 97 and 99 to 101, or a pharmaceutically acceptable salt thereof, wherein R² is —CF₃.
Embodiment 115. The compound of any one of embodiments 1 to 97 and 99 to 101, or a pharmaceutically acceptable salt thereof, wherein R² is —OCH₃.
Embodiment 116. The compound of any one of embodiments 1 to 115, or a pharmaceutically acceptable salt thereof, wherein Ring B is selected from the group consisting of C₆-C₁₀ aryl and 5-10 membered heteroaryl, wherein the aryl and heteroaryl are optionally substituted at any available position.
Embodiment 117. The compound of any one of embodiments 1 to 115, or a pharmaceutically acceptable salt thereof, wherein Ring B is selected from the group consisting of phenyl, naphthalenyl, C₃-C₇ monocyclic cycloalkyl, 3-7 membered monocyclic heterocyclyl, 5-6 membered monocyclic heteroaryl, and 8-10 membered bicyclic heteroaryl, each optionally substituted at any available position.
Embodiment 118. The compound of any one of embodiments 1 to 115, or a pharmaceutically acceptable salt thereof, wherein Ring B is selected from the group consisting of C₆-C₁₀ aryl and 8-10 membered bicyclic heteroaryl wherein the aryl and heteroaryl are optionally substituted at any available position.
Embodiment 119. The compound of any one of embodiments 1 to 115, or a pharmaceutically acceptable salt thereof, wherein Ring B is selected from the group consisting of cyclopropyl, cyclobutyl, cyclopentyl, cyclohexyl, cycloheptyl, oxetanyl, tetrahydrofuranyl, tetrahydropyranyl, azetidinyl, pyrrolidinyl, piperidinyl, piperazinyl, thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, isoquinolinyl, 1,2,3,4-tetrahydroquinolinyl, 1,2,3,4-tetrahydroisoquinolinyl, 2,3-dihydrobenzofuranyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted at any available position.
Embodiment 120. The compound of any one of embodiments 1 to 115, or a pharmaceutically acceptable salt thereof, wherein Ring B is selected from the group consisting of cyclopropyl, cyclobutyl, cyclopentyl, cyclohexyl, cycloheptyl, oxetanyl, tetrahydrofuranyl, tetrahydropyranyl, azetidinyl, pyrrolidinyl, piperidinyl, piperazinyl, thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each optionally substituted at any available position.
Embodiment 121. The compound of any one of embodiments 1 to 115, or a pharmaceutically acceptable salt thereof, wherein Ring B is selected from the group consisting of cyclopropyl, cyclobutyl, cyclopentyl, cyclohexyl, tetrahydrofuranyl, tetrahydropyranyl, thiophenyl, oxazolyl, pyrazolyl, phenyl, naphthalenyl, pyridinyl, 1H-indazolyl, 2H-indazolyl, quinolinyl, isoquinolinyl, 1,2,3,4-tetrahydroquinolinyl, 1,2,3,4-tetrahydroisoquinolinyl, 2,3-dihydrobenzofuranyl and benzo[d]thiazolyl, each optionally substituted at any available position.
Embodiment 122. The compound any one of embodiments 1 to 115, or a pharmaceutically acceptable salt thereof, wherein Ring B is selected from the group consisting of cyclobutyl, cyclopentyl, tetrahydropyranyl, pyrazolyl, thiophenyl, oxazolyl, pyridinyl, phenyl, naphthalenyl, 1H-indazolyl, 2H-indazolyl, quinolinyl, isoquinolinyl and benzo[d]thiazolyl, each optionally substituted at any available position.
Embodiment 123. The compound any one of embodiments 1 to 115, or a pharmaceutically acceptable salt thereof, wherein Ring B is selected from the group consisting of cyclopentyl, tetrahydropyranyl, pyrazolyl, thiophenyl, oxazolyl, pyridinyl, phenyl, naphthalenyl, 2H-indazolyl, quinolinyl, isoquinolinyl and benzo[d]thiazolyl, each optionally substituted at any available position.
Embodiment 124. The compound of any one of embodiments 1 to 115, or a pharmaceutically acceptable salt thereof, wherein Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted at any available position.
Embodiment 125. The compound of any one of embodiments 1 to 115, or a pharmaceutically acceptable salt thereof, wherein Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-3-yl, tetrahydropyran-4-yl, pyrazol-5-yl, pyrazol-3-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted at any available position.
Embodiment 126. The compound of any one of embodiments 1 to 115, or a pharmaceutically acceptable salt thereof, wherein Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-3-yl, tetrahydropyran-4-yl, pyrazol-5-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted at any available position.
Embodiment 127. The compound of any one of embodiments 1 to 115, or a pharmaceutically acceptable salt thereof, wherein Ring B is selected from the group consisting of cyclopropyl, cyclobutyl, cyclopentyl, cyclohexyl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H indazol-5-yl, 1H indazol-4-yl, 2H-indazol-6-yl, 2H-indazol-5-yl, quinolin-6-yl, quinolin-7-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl and benzo[d]thiazol-5-yl, each optionally substituted at any available position.
Embodiment 128. The compound of any one of embodiments 1 to 115, or a pharmaceutically acceptable salt thereof, wherein Ring B is C₃-C₇ monocyclic cycloalkyl, optionally substituted at any available position.
Embodiment 129. The compound of any one of embodiments 1 to 115, or a pharmaceutically acceptable salt thereof, wherein Ring B is selected from the group consisting of cyclopropyl, cyclobutyl, cyclopentyl and cyclohexyl, each optionally substituted at any available position.
Embodiment 130. The compound of any one of embodiments 1 to 115, or a pharmaceutically acceptable salt thereof, wherein Ring B is cyclopropyl, optionally substituted at any available position.
Embodiment 131. The compound of any one of embodiments 1 to 115, or a pharmaceutically acceptable salt thereof, wherein Ring B is cyclobutyl, optionally substituted at any available position.
Embodiment 132. The compound of any one of embodiments 1 to 115, or a pharmaceutically acceptable salt thereof, wherein Ring B is cyclopentyl, optionally substituted at any available position.
Embodiment 133. The compound of any one of embodiments 1 to 115, or a pharmaceutically acceptable salt thereof, wherein Ring B is cyclohexyl, optionally substituted at any available position.
Embodiment 134. The compound of any one of embodiments 1 to 115, or a pharmaceutically acceptable salt thereof, wherein Ring B is 3-7 membered monocyclic heterocyclyl containing 1 or 2 heteroatoms independently selected from N, O and S or oxidized forms thereof, wherein the heterocyclyl is optionally substituted at any available position.
Embodiment 135. The compound of any one of embodiments 1 to 115, or a pharmaceutically acceptable salt thereof, wherein Ring B is selected from the group consisting of tetrahydrofuranyl and tetrahydropyranyl, each optionally substituted at any available position.
Embodiment 136. The compound of any one of embodiments 1 to 115, or a pharmaceutically acceptable salt thereof, wherein Ring B is selected from the group consisting of tetrahydrofuran-3-yl, tetrahydropyran-2-yl, tetrahydropyran-3-yl and tetrahydropyran-4-yl, each optionally substituted at any available position.
Embodiment 137. The compound of any one of embodiments 1 to 115, or a pharmaceutically acceptable salt thereof, wherein Ring B is tetrahydrofuranyl, optionally substituted at any available position.
Embodiment 138. The compound of any one of embodiments 1 to 115, or a pharmaceutically acceptable salt thereof, wherein Ring B is tetrahydrofuran-3-yl, optionally substituted at any available position.
Embodiment 139. The compound of any one of embodiments 1 to 115, or a pharmaceutically acceptable salt thereof, wherein Ring B is tetrahydropyranyl, optionally substituted at any available position.
Embodiment 140. The compound of any one of embodiments 1 to 115, or a pharmaceutically acceptable salt thereof, wherein Ring B is tetrahydropyran-2-yl, optionally substituted at any available position.
Embodiment 141. The compound of any one of embodiments 1 to 115, or a pharmaceutically acceptable salt thereof, wherein Ring B is tetrahydropyran-3-yl, optionally substituted at any available position.
Embodiment 142. The compound of any one of embodiments 1 to 115, or a pharmaceutically acceptable salt thereof, wherein Ring B is tetrahydropyran-4-yl, optionally substituted at any available position.
Embodiment 143. The compound of any one of embodiments 1 to 115, or a pharmaceutically acceptable salt thereof, wherein Ring B is a 5-6 membered monocyclic heteroaryl containing 1, 2 or 3 heteroatoms independently selected from N, O and S, optionally substituted at any available position.
Embodiment 144. The compound of any one of embodiments 1 to 115, or a pharmaceutically acceptable salt thereof, wherein Ring B is selected from the group consisting of pyrazolyl, thiophenyl, oxazolyl and pyridinyl, each optionally substituted at any available position.
Embodiment 145. The compound of any one of embodiments 1 to 115, or a pharmaceutically acceptable salt thereof, wherein Ring B is selected from the group consisting of pyrazol-5-yl, pyrazol-3-yl, thiophen-3-yl, oxazol-5-yl, pyridin-3-yl and pyridin-4-yl, each optionally substituted at any available position.
Embodiment 146. The compound of any one of embodiments 1 to 115, or a pharmaceutically acceptable salt thereof, wherein Ring B is pyrazolyl, optionally substituted at any available position.
Embodiment 147. The compound of any one of embodiments 1 to 115, or a pharmaceutically acceptable salt thereof, wherein Ring B is pyrazol-5-yl, optionally substituted at any available position.
Embodiment 148. The compound of any one of embodiments 1 to 115, or a pharmaceutically acceptable salt thereof, wherein Ring B is pyrazol-3-yl, optionally substituted at any available position.
Embodiment 149. The compound of any one of embodiments 1 to 115, or a pharmaceutically acceptable salt thereof, wherein Ring B is thiophenyl, optionally substituted at any available position.
Embodiment 150. The compound of any one of embodiments 1 to 115, or a pharmaceutically acceptable salt thereof, wherein Ring B is thiophen-3-yl, optionally substituted at any available position.
Embodiment 151. The compound of any one of embodiments 1 to 115, or a pharmaceutically acceptable salt thereof, wherein Ring B is oxazolyl, optionally substituted at any available position.
Embodiment 152. The compound of any one of embodiments 1 to 115, or a pharmaceutically acceptable salt thereof, wherein Ring B is oxazol-5-yl, optionally substituted at any available position.
Embodiment 153. The compound of any one of embodiments 1 to 115, or a pharmaceutically acceptable salt thereof, wherein Ring B is pyridinyl, optionally substituted at any available position.
Embodiment 154. The compound of any one of embodiments 1 to 115, or a pharmaceutically acceptable salt thereof, wherein Ring B is pyridin-3-yl, optionally substituted at any available position.
Embodiment 155. The compound of any one of embodiments 1 to 115, or a pharmaceutically acceptable salt thereof, wherein Ring B is pyridin-4-yl, optionally substituted at any available position.
Embodiment 156. The compound any one of embodiments 1 to 115, or a pharmaceutically acceptable salt thereof, wherein Ring B is selected from the group consisting of cyclopentyl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H-indazol-5-yl, 2H-indazol-6-yl, 2H-indazol-5-yl, quinolin-6-yl, quinolin-7-yl, isoquinolin-6-yl, benzo[d]thiazol-5-yl, each optionally substituted.
Embodiment 157. The compound any one of embodiments 1 to 115, or a pharmaceutically acceptable salt thereof, wherein Ring B is selected from the group consisting of cyclopentyl, tetrahydropyran-3-yl, tetrahydropyran-4-yl, pyrazol-5-yl, thiophen-3-yl, oxazol-5-yl, pyridin-3-yl, pyridin-4-yl, phenyl, naphthalen-2-yl, 2H-indazol-6-yl, 2H-indazol-5-yl, quinolin-6-yl, quinolin-7-yl, isoquinolin-6-yl and benzo[d]thiazol-5-yl, each optionally substituted at any available position.
Embodiment 158. The compound of any one of embodiments 1 to 115, or a pharmaceutically acceptable salt thereof, wherein Ring B is C₆-C₁₀ aryl (e.g., naphthalenyl, phenyl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl), wherein the aryl is optionally substituted at any available position.
Embodiment 159. The compound of any one of embodiments 1 to 115, or a pharmaceutically acceptable salt thereof, wherein Ring B is selected from the group consisting of naphthalenyl, phenyl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each optionally substituted at any available position.
Embodiment 160. The compound of any one of embodiments 1 to 115, or a pharmaceutically acceptable salt thereof, wherein Ring B is selected from the group consisting of phenyl, naphthalen-2-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each optionally substituted at any available position.
Embodiment 161. The compound of any one of embodiments 1 to 115, or a pharmaceutically acceptable salt thereof, wherein Ring B is naphthalenyl or phenyl, each optionally substituted at any available position.
Embodiment 162. The compound of any one of embodiments 1 to 115, or a pharmaceutically acceptable salt thereof, wherein Ring B is naphthalenyl, optionally substituted at any available position.
Embodiment 163. The compound of any one of embodiments 1 to 115, or a pharmaceutically acceptable salt thereof, wherein Ring B is selected from the group consisting of naphthalen-1-yl and naphthalen-2-yl, each optionally substituted at any available position.
Embodiment 164. The compound of any one of embodiments 1 to 115, or a pharmaceutically acceptable salt thereof, wherein Ring B is naphthalen-2-yl optionally substituted at any available position.
Embodiment 165. The compound of any one of embodiments 1 to 115, or a pharmaceutically acceptable salt thereof, wherein Ring B is phenyl, optionally substituted at any available position.
Embodiment 166. The compound of any one of embodiments 1 to 115, or a pharmaceutically acceptable salt thereof, wherein Ring B is 1,2,3,4-tetrahydroquinolin-6-yl, optionally substituted at any available position.
Embodiment 167. The compound of any one of embodiments 1 to 115, or a pharmaceutically acceptable salt thereof, wherein Ring B is 1,2,3,4-tetrahydroisoquinolin-6-yl, optionally substituted at any available position.
Embodiment 168. The compound of any one of embodiments 1 to 115, or a pharmaceutically acceptable salt thereof, wherein Ring B is 1,2,3,4-tetrahydroisoquinolin-7-yl, optionally substituted at any available position.
Embodiment 169. The compound of any one of embodiments 1 to 115, or a pharmaceutically acceptable salt thereof, wherein Ring B is 2,3-dihydrobenzofuran-4-yl, optionally substituted at any available position.
Embodiment 170. The compound of any one of embodiments 1 to 115, or a pharmaceutically acceptable salt thereof, wherein Ring B is 2,3-dihydrobenzofuran-5-yl, optionally substituted at any available position.
Embodiment 171. The compound of any one of embodiments 1 to 115, or a pharmaceutically acceptable salt thereof, wherein Ring B is 2,3-dihydrobenzofuran-7-yl, optionally substituted at any available position.
Embodiment 172. The compound of any one of embodiments 1 to 115, or a pharmaceutically acceptable salt thereof, wherein Ring B is an 8-10 membered bicyclic heteroaryl, wherein the bicyclic heteroaryl is optionally substituted at any available position.
Embodiment 173. The compound of any one of embodiments 1 to 115, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted at any available position.
Embodiment 174. The compound of any one of embodiments 1 to 115, or a pharmaceutically acceptable salt thereof, wherein Ring B is selected from the group consisting of 1H-indazolyl, 2H-indazolyl, quinolinyl, isoquinolinyl and benzo[d]thiazolyl, each optionally substituted at any available position.
Embodiment 175. The compound of any one of embodiments 1 to 115, or a pharmaceutically acceptable salt thereof, wherein Ring B is selected from the group consisting of 2H-indazolyl, quinolinyl, isoquinolinyl and benzo[d]thiazolyl, each optionally substituted at any available position.
Embodiment 176. The compound of any one of embodiments 1 to 115, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each optionally substituted at any available position.
Embodiment 177. The compound of any one of embodiments 1 to 115, or a pharmaceutically acceptable salt thereof, wherein Ring B is independently selected from the group consisting of 1H-indazol-5-yl, 1H indazol-4-yl, 2H-indazol-6-yl, 2H-indazol-5-yl, quinolin-6-yl, quinolin-7-yl, isoquinolin-6-yl and benzo[d]thiazol-5-yl, each optionally substituted at any available position.
Embodiment 178. The compound of any one of embodiments 1 to 115, or a pharmaceutically acceptable salt thereof, wherein Ring B is independently selected from the group consisting of 1H-indazol-5-yl, 2H-indazol-6-yl, 2H-indazol-5-yl, quinolin-6-yl, quinolin-7-yl, isoquinolin-6-yl and benzo[d]thiazol-5-yl, each optionally substituted at any available position.
Embodiment 179. The compound of any one of embodiments 1 to 115, or a pharmaceutically acceptable salt thereof, wherein Ring B is independently selected from the group consisting of 2H-indazol-6-yl, 2H-indazol-5-yl, quinolin-6-yl, quinolin-7-yl, isoquinolin-6-yl and benzo[d]thiazol-5-yl, each optionally substituted at any available position.
Embodiment 180. The compound of any one of embodiments 1 to 115, or a pharmaceutically acceptable salt thereof, wherein Ring B is 1H indazolyl, optionally substituted at any available position.
Embodiment 181. The compound of any one of embodiments 1 to 115, or a pharmaceutically acceptable salt thereof, wherein Ring B is 1H indazol-5-yl, optionally substituted at any available position.
Embodiment 182. The compound of any one of embodiments 1 to 115, or a pharmaceutically acceptable salt thereof, wherein Ring B is 1H indazol-4-yl, optionally substituted at any available position.
Embodiment 183. The compound of any one of embodiments 1 to 115, or a pharmaceutically acceptable salt thereof, wherein Ring B is 2H-indazolyl, optionally substituted at any available position.
Embodiment 184. The compound of any one of embodiments 1 to 115, or a pharmaceutically acceptable salt thereof, wherein Ring B is 2H-indazol-6-yl.
Embodiment 185. The compound of any one of embodiments 1 to 115, or a pharmaceutically acceptable salt thereof, wherein Ring B is 2H-indazol-5-yl, optionally substituted at any available position.
Embodiment 186. The compound of any one of embodiments 1 to 115, or a pharmaceutically acceptable salt thereof, wherein Ring B is quinolinyl, optionally substituted at any available position.
Embodiment 187. The compound of any one of embodiments 1 to 115, or a pharmaceutically acceptable salt thereof, wherein Ring B is quinolin-6-yl.
Embodiment 188. The compound of any one of embodiments 1 to 115, or a pharmaceutically acceptable salt thereof, wherein Ring B is quinolin-7-yl, optionally substituted at any available position.
Embodiment 189. The compound of any one of embodiments 1 to 115, or a pharmaceutically acceptable salt thereof, wherein Ring B is isoquinolinyl, optionally substituted at any available position.
Embodiment 190. The compound of any one of embodiments 1 to 115, or a pharmaceutically acceptable salt thereof, wherein Ring B is isoquinolin-6-yl, optionally substituted at any available position.
Embodiment 191. The compound of any one of embodiments 1 to 115, or a pharmaceutically acceptable salt thereof, wherein Ring B is and benzo[d]thiazolyl, optionally substituted at any available position.
Embodiment 192. The compound of any one of embodiments 1 to 115, or a pharmaceutically acceptable salt thereof, wherein Ring B is and benzo[d]thiazol-5-yl, optionally substituted at any available position.
Embodiment 193. The compound of any one of embodiments 1 to 192, or a pharmaceutically acceptable salt thereof, wherein Ring B is substituted at available positions with 0, 1, 2 or 3 instances of R³;
each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3}) R^{a3}, —NR^{a3}S(═O)₂R^{a3} and —S(═O)₂N(R^{a3})₂, wherein each alkyl, cycloalkyl, heteroalkyl, heterocyclyl, aryl, heteroaryl, cycloalkylalkyl, heterocyclylalkyl, arylalkyl and heteroarylalkyl of R³ is optionally substituted at any available position (e.g., substituted with 0, 1, 2, 3, 4, or 5 instances of R⁶, wherein each R⁶ is independently selected from the group consisting of —C₁-C₆ alkyl, —C₃-C₉ cycloalkyl, 3-10 membered heterocyclyl, —OH, ═O, halo, —OC₁-C₆ alkyl, —C₁-C₆ haloalkyl, —C(═O)C₁-C₆ alkyl, —N(C₁-C₆ alkyl)₂, —C₁-C₆ heteroalkyl, and —NHC(═O)C₁-C₆ alkyl, wherein one or more hydrogens of the —C₁-C₆ alkyl can be replaced with deuterium); and
each R^{a3} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl).
Embodiment 194. The compound of embodiment 193, or a pharmaceutically acceptable salt thereof, wherein Ring B is selected from the group consisting of C₆-C₁₀ aryl and 5-10 membered heteroaryl, wherein the aryl and heteroaryl are substituted at available positions with 0, 1, 2 or 3 instances of R³.
Embodiment 195. The compound of embodiment 193, or a pharmaceutically acceptable salt thereof, wherein Ring B is selected from the group consisting of phenyl, naphthalenyl, C₃-C₇ monocyclic cycloalkyl, 3-7 membered monocyclic heterocyclyl, 5-6 membered monocyclic heteroaryl, and 8-10 membered bicyclic heteroaryl, each substituted at available positions with 0, 1, 2 or 3 instances of R³.
Embodiment 196. The compound of embodiment 193, or a pharmaceutically acceptable salt thereof, wherein Ring B is selected from the group consisting of C₆-C₁₀ aryl and 8-10 membered bicyclic heteroaryl wherein the aryl and heteroaryl are substituted at available positions with 0, 1, 2 or 3 instances of R³.
Embodiment 197. The compound of embodiment 193, or a pharmaceutically acceptable salt thereof, wherein Ring B is selected from the group consisting of cyclopropyl, cyclobutyl, cyclopentyl, cyclohexyl, cycloheptyl, oxetanyl, tetrahydrofuranyl, tetrahydropyranyl, azetidinyl, pyrrolidinyl, piperidinyl, piperazinyl, thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, isoquinolinyl, 1,2,3,4-tetrahydroquinolinyl, 1,2,3,4-tetrahydroisoquinolinyl, 2,3-dihydrobenzofuranyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each substituted at available positions with 0, 1, 2 or 3 instances of R³.
Embodiment 198. The compound of embodiment 193, or a pharmaceutically acceptable salt thereof, wherein Ring B is selected from the group consisting of cyclopropyl, cyclobutyl, cyclopentyl, cyclohexyl, cycloheptyl, oxetanyl, tetrahydrofuranyl, tetrahydropyranyl, azetidinyl, pyrrolidinyl, piperidinyl, piperazinyl, thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each substituted with 0, 1, 2 or 3 instances of R³.
Embodiment 199. The compound of embodiment 193, or a pharmaceutically acceptable salt thereof, wherein Ring B is selected from the group consisting of cyclopropyl, cyclobutyl, cyclopentyl, cyclohexyl, tetrahydrofuranyl, tetrahydropyranyl, thiophenyl, oxazolyl, pyrazolyl, phenyl, naphthalenyl, pyridinyl, 1H-indazolyl, 2H-indazolyl, quinolinyl, isoquinolinyl, 1,2,3,4-tetrahydroquinolinyl, 1,2,3,4-tetrahydroisoquinolinyl, 2,3-dihydrobenzofuranyl and benzo[d]thiazolyl, each substituted at available positions with 0, 1, 2 or 3 instances of R³.
Embodiment 200. The compound of embodiment 193, or a pharmaceutically acceptable salt thereof, wherein Ring B is selected from the group consisting of cyclobutyl, cyclopentyl, tetrahydropyranyl, pyrazolyl, thiophenyl, oxazolyl, pyridinyl, phenyl, naphthalenyl, 1H-indazolyl, 2H-indazolyl, quinolinyl, isoquinolinyl and benzo[d]thiazolyl, each substituted with 0, 1, 2 or 3 instances of R³.
Embodiment 201. The compound of embodiment 193, or a pharmaceutically acceptable salt thereof, wherein Ring B is selected from the group consisting of cyclopentyl, tetrahydropyranyl, pyrazolyl, thiophenyl, oxazolyl, pyridinyl, phenyl, naphthalenyl, 2H-indazolyl, quinolinyl, isoquinolinyl and benzo[d]thiazolyl, each substituted with 0, 1, 2 or 3 instances of R³.
Embodiment 202. The compound of embodiment 193, or a pharmaceutically acceptable salt thereof, wherein Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each substituted at available positions with 0, 1, 2 or 3 instances of R³.
Embodiment 203. The compound of embodiment 193, or a pharmaceutically acceptable salt thereof, wherein Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-3-yl, tetrahydropyran-4-yl, pyrazol-5-yl, pyrazol-3-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each substituted with 0, 1, 2 or 3 instances of R³.
Embodiment 204. The compound of embodiment 193, or a pharmaceutically acceptable salt thereof, wherein Ring B is selected from the group consisting of cyclopropyl, cyclobutyl, cyclopentyl, cyclohexyl, oxetan-3-yl, tetrahydrofuran-2-yl, tetrahydrofuran-3-yl, tetrahydropyran-3-yl, tetrahydropyran-4-yl, pyrazol-5-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each substituted with 0, 1, 2 or 3 instances of R³.
Embodiment 205. The compound embodiment 193, or a pharmaceutically acceptable salt thereof, wherein Ring B is selected from the group consisting of cyclopentyl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 2H-indazol-6-yl, 1H-indazol-5-yl, 2H-indazol-5-yl, quinolin-6-yl, quinolin-7-yl, isoquinolin-6-yl, benzo[d]thiazol-5-yl, each substituted with 0, 1, 2 or 3 instances of R³.
Embodiment 206. The compound embodiment 193, or a pharmaceutically acceptable salt thereof, wherein Ring B is selected from the group consisting of cyclopentyl, tetrahydropyran-3-yl, tetrahydropyran-4-yl, pyrazol-5-yl, thiophen-3-yl, oxazol-5-yl, pyridin-3-yl, pyridin-4-yl, phenyl, naphthalen-2-yl, 2H-indazol-6-yl, 2H-indazol-5-yl, quinolin-6-yl, quinolin-7-yl, isoquinolin-6-yl, benzo[d]thiazol-5-yl, each substituted with 0, 1, 2 or 3 instances of R³.
Embodiment 207. The compound of embodiment 193, or a pharmaceutically acceptable salt thereof, wherein Ring B is selected from the group consisting of cyclopropyl, cyclobutyl, cyclopentyl, cyclohexyl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H indazol-5-yl, 1H indazol-4-yl, 2H-indazol-6-yl, 2H-indazol-5-yl, quinolin-6-yl, quinolin-7-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl and benzo[d]thiazol-5-yl, each substituted at available positions with 0, 1, 2 or 3 instances of R³.
Embodiment 208. The compound of embodiment 193, or a pharmaceutically acceptable salt thereof, wherein Ring B is C₃-C₇ monocyclic cycloalkyl, substituted at available positions with 0, 1, 2 or 3 instances of R³.
Embodiment 209. The compound of embodiment 193, or a pharmaceutically acceptable salt thereof, wherein Ring B is selected from the group consisting of cyclopropyl, cyclobutyl, cyclopentyl and cyclohexyl, each substituted at available positions with 0, 1, 2 or 3 instances of R³.
Embodiment 210. The compound of embodiment 193, or a pharmaceutically acceptable salt thereof, wherein Ring B is cyclopropyl, substituted at available positions with 0, 1, 2 or 3 instances of R³.
Embodiment 211. The compound of embodiment 193, or a pharmaceutically acceptable salt thereof, wherein Ring B is cyclobutyl, substituted at available positions with 0, 1, 2 or 3 instances of R³.
Embodiment 212. The compound of embodiment 193, or a pharmaceutically acceptable salt thereof, wherein Ring B is cyclopentyl, substituted at available positions with 0, 1, 2 or 3 instances of R³.
Embodiment 213. The compound of embodiment 193, or a pharmaceutically acceptable salt thereof, wherein Ring B is cyclohexyl, substituted at available positions with 0, 1, 2 or 3 instances of R³.
Embodiment 214. The compound of embodiment 193, or a pharmaceutically acceptable salt thereof, wherein Ring B is 3-7 membered monocyclic heterocyclyl containing 1 or 2 heteroatoms independently selected from N, O and S or oxidized forms thereof, wherein the heterocyclyl is substituted at available positions with 0, 1, 2 or 3 instances of R³.
Embodiment 215. The compound of embodiment 193, or a pharmaceutically acceptable salt thereof, wherein Ring B is selected from the group consisting of tetrahydrofuranyl and tetrahydropyranyl, each substituted at available positions with 0, 1, 2 or 3 instances of R³.
Embodiment 216. The compound of embodiment 193, or a pharmaceutically acceptable salt thereof, wherein Ring B is selected from the group consisting of tetrahydrofuran-3-yl, tetrahydropyran-2-yl, tetrahydropyran-3-yl and tetrahydropyran-4-yl, each substituted at available positions with 0, 1, 2 or 3 instances of R³.
Embodiment 217. The compound of embodiment 193, or a pharmaceutically acceptable salt thereof, wherein Ring B is tetrahydrofuranyl, substituted at available positions with 0, 1, 2 or 3 instances of R³.
Embodiment 218. The compound of embodiment 193, or a pharmaceutically acceptable salt thereof, wherein Ring B is tetrahydrofuran-3-yl, substituted at available positions with 0, 1, 2 or 3 instances of R³.
Embodiment 219. The compound of embodiment 193, or a pharmaceutically acceptable salt thereof, wherein Ring B is tetrahydropyranyl, substituted at available positions with 0, 1, 2 or 3 instances of R³.
Embodiment 220. The compound of embodiment 193, or a pharmaceutically acceptable salt thereof, wherein Ring B is tetrahydropyran-2-yl, substituted at available positions with 0, 1, 2 or 3 instances of R³.
Embodiment 221. The compound of embodiment 193, or a pharmaceutically acceptable salt thereof, wherein Ring B is tetrahydropyran-3-yl, substituted at available positions with 0, 1, 2 or 3 instances of R³.
Embodiment 222. The compound of embodiment 193, or a pharmaceutically acceptable salt thereof, wherein Ring B is tetrahydropyran-4-yl, substituted at available positions with 0, 1, 2 or 3 instances of R³.
Embodiment 223. The compound of embodiment 193, or a pharmaceutically acceptable salt thereof, wherein Ring B is a 5-6 membered monocyclic heteroaryl containing 1, 2 or 3 heteroatoms independently selected from N, O and S, substituted at available positions with 0, 1, 2 or 3 instances of R³.
Embodiment 224. The compound of embodiment 193, or a pharmaceutically acceptable salt thereof, wherein Ring B is selected from the group consisting of pyrazolyl, thiophenyl, oxazolyl and pyridinyl, each substituted at available positions with 0, 1, 2 or 3 instances of R³.
Embodiment 225. The compound of embodiment 193, or a pharmaceutically acceptable salt thereof, wherein Ring B is selected from the group consisting of pyrazol-5-yl, pyrazol-3-yl, thiophen-3-yl, oxazol-5-yl, pyridin-3-yl and pyridin-4-yl, each substituted at available positions with 0, 1, 2 or 3 instances of R³.
Embodiment 226. The compound of embodiment 193, or a pharmaceutically acceptable salt thereof, wherein Ring B is pyrazolyl, substituted at available positions with 0, 1, 2 or 3 instances of R³.
Embodiment 227. The compound of embodiment 193, or a pharmaceutically acceptable salt thereof, wherein Ring B is pyrazol-5-yl, substituted at available positions with 0, 1, 2 or 3 instances of R³.
Embodiment 228. The compound of embodiment 193, or a pharmaceutically acceptable salt thereof, wherein Ring B is pyrazol-3-yl, substituted at available positions with 0, 1, 2 or 3 instances of R³
Embodiment 229. The compound of embodiment 193, or a pharmaceutically acceptable salt thereof, wherein Ring B is thiophenyl, substituted at available positions with 0, 1, 2 or 3 instances of R³.
Embodiment 230. The compound of embodiment 193, or a pharmaceutically acceptable salt thereof, wherein Ring B is thiophen-3-yl, substituted at available positions with 0, 1, 2 or 3 instances of R³.
Embodiment 231. The compound of embodiment 193, or a pharmaceutically acceptable salt thereof, wherein Ring B is oxazolyl, substituted at available positions with 0, 1, 2 or 3 instances of R³.
Embodiment 232. The compound of embodiment 193, or a pharmaceutically acceptable salt thereof, wherein Ring B is oxazol-5-yl, substituted at available positions with 0, 1, 2 or 3 instances of R³.
Embodiment 233. The compound of embodiment 193, or a pharmaceutically acceptable salt thereof, wherein Ring B is pyridinyl, substituted at available positions with 0, 1, 2 or 3 instances of R³.
Embodiment 234. The compound of embodiment 193, or a pharmaceutically acceptable salt thereof, wherein Ring B is pyridin-3-yl, substituted at available positions with 0, 1, 2 or 3 instances of R³.
Embodiment 235. The compound of embodiment 193, or a pharmaceutically acceptable salt thereof, wherein Ring B is pyridin-4-yl, substituted at available positions with 0, 1, 2 or 3 instances of R³.
Embodiment 236. The compound of embodiment 193, or a pharmaceutically acceptable salt thereof, wherein Ring B is C₆-C₁₀ aryl (e.g., naphthalenyl, phenyl, 1,2,3,4-tetrahydroquinolinyl, 1,2,3,4-tetrahydroisoquinolinyl, 2,3-dihydrobenzofuranyl), wherein the aryl is substituted at available positions with 0, 1, 2 or 3 instances of R³.
Embodiment 237. The compound of embodiment 193, or a pharmaceutically acceptable salt thereof, wherein Ring B is selected from the group consisting of naphthalenyl, phenyl, 1,2,3,4-tetrahydroquinolinyl, 1,2,3,4-tetrahydroisoquinolinyl, 2,3-dihydrobenzofuranyl), wherein the aryl is substituted at available positions with 0, 1, 2 or 3 instances of R³
Embodiment 238. The compound of embodiment 193, or a pharmaceutically acceptable salt thereof, wherein Ring B is selected from the group consisting of phenyl, naphthalen-2-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each substituted at available positions with 0, 1, 2 or 3 instances of R³.
Embodiment 239. The compound of embodiment 193, or a pharmaceutically acceptable salt thereof, wherein Ring B is naphthalenyl or phenyl, each substituted at available positions with 0, 1, 2 or 3 instances of R³.
Embodiment 240. The compound of embodiment 193, wherein Ring B is naphthalenyl, substituted at available positions with 0, 1, 2 or 3 instances of R³.
Embodiment 241. The compound of embodiment 193, or a pharmaceutically acceptable salt thereof, wherein Ring B is selected from the group consisting of naphthalen-1-yl and naphthalen-2-yl, each substituted at available positions with 0, 1, 2 or 3 instances of R³.
Embodiment 242. The compound of embodiment 193, or a pharmaceutically acceptable salt thereof, wherein Ring B is naphthalen-2-yl substituted at available positions with 0, 1, 2 or 3 instances of R³.
Embodiment 243. The compound of embodiment 193, or a pharmaceutically acceptable salt thereof, wherein Ring B is phenyl substituted at available positions with 0, 1, 2 or 3 instances of R³.
Embodiment 244. The compound of embodiment 193, or a pharmaceutically acceptable salt thereof, wherein Ring B is 1,2,3,4-tetrahydroquinolinyl, substituted at available positions with 0, 1, 2 or 3 instances of R³.
Embodiment 245. The compound of embodiment 193, or a pharmaceutically acceptable salt thereof, wherein Ring B is 1,2,3,4-tetrahydroquinolin-6-yl, substituted at available positions with 0, 1, 2 or 3 instances of R³.
Embodiment 246. The compound of embodiment 193, or a pharmaceutically acceptable salt thereof, wherein Ring B is 1,2,3,4-tetrahydroisoquinolinyl, substituted at available positions with 0, 1, 2 or 3 instances of R³.
Embodiment 247. The compound of embodiment 193, or a pharmaceutically acceptable salt thereof, wherein Ring B is 1,2,3,4-tetrahydroisoquinolin-6-yl, substituted at available positions with 0, 1, 2 or 3 instances of R³.
Embodiment 248. The compound of embodiment 193, or a pharmaceutically acceptable salt thereof, wherein Ring B is 1,2,3,4-tetrahydroisoquinolin-7-yl, substituted at available positions with 0, 1, 2 or 3 instances of R³.
Embodiment 249. The compound of embodiment 193, or a pharmaceutically acceptable salt thereof, wherein Ring B is 2,3-dihydrobenzofuranyl, substituted at available positions with 0, 1, 2 or 3 instances of R³.
Embodiment 250. The compound of embodiment 193, or a pharmaceutically acceptable salt thereof, wherein Ring B is 2,3-dihydrobenzofuran-4-yl, substituted at available positions with 0, 1, 2 or 3 instances of R³.
Embodiment 251. The compound of embodiment 193, or a pharmaceutically acceptable salt thereof, wherein Ring B is 2,3-dihydrobenzofuran-5-yl, substituted at available positions with 0, 1, 2 or 3 instances of R³.
Embodiment 252. The compound of embodiment 193, or a pharmaceutically acceptable salt thereof, wherein Ring B is 2,3-dihydrobenzofuran-7-yl, substituted at available positions with 0, 1, 2 or 3 instances of R³.
Embodiment 253. The compound of embodiment 193, or a pharmaceutically acceptable salt thereof, wherein Ring B is an 8-10 membered bicyclic heteroaryl, wherein the bicyclic heteroaryl is substituted with 0, 1, 2 or 3 instances of R³.
Embodiment 254. The compound of embodiment 193, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each substituted with 0, 1, 2 or 3 instances of R³.
Embodiment 255. The compound of embodiment 193, or a pharmaceutically acceptable salt thereof, wherein Ring B is selected from the group consisting of 1H-indazolyl, 2H-indazolyl, quinolinyl, isoquinolinyl and benzo[d]thiazolyl, each substituted with 0, 1, 2 or 3 instances of R³.
Embodiment 256. The compound of embodiment 193, or a pharmaceutically acceptable salt thereof, wherein Ring B is selected from the group consisting of 2H-indazolyl, quinolinyl, isoquinolinyl and benzo[d]thiazolyl, each substituted with 0, 1, 2 or 3 instances of R³.
Embodiment 257. The compound of embodiment 193, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each substituted with 0, 1, 2 or 3 instances of R³.
Embodiment 258. The compound of embodiment 193, or a pharmaceutically acceptable salt thereof, wherein Ring B is independently selected from the group consisting of 1H-indazol-5-yl, 1H indazol-4-yl, 2H-indazol-6-yl, 2H-indazol-5-yl, quinolin-6-yl, quinolin-7-yl, isoquinolin-6-yl and benzo[d]thiazol-5-yl, each substituted at available positions with 0, 1, 2 or 3 instances of R³.
Embodiment 259. The compound of embodiment 193, or a pharmaceutically acceptable salt thereof, wherein Ring B is selected from the group consisting of 1H-indazol-5-yl, 2H-indazol-6-yl, 2H-indazol-5-yl, quinolin-6-yl, quinolin-7-yl, isoquinolin-6-yl and benzo[d]thiazol-5-yl, each substituted with 0, 1, 2 or 3 instances of R³.
Embodiment 260. The compound of embodiment 193, or a pharmaceutically acceptable salt thereof, wherein Ring B is selected from the group consisting of 2H-indazol-6-yl, 2H-indazol-5-yl, quinolin-6-yl, quinolin-7-yl, isoquinolin-6-yl and benzo[d]thiazol-5-yl, each substituted with 0, 1, 2 or 3 instances of R³.
Embodiment 261. The compound of embodiment 193, or a pharmaceutically acceptable salt thereof, wherein Ring B is 1H indazolyl, substituted at available positions with 0, 1, 2 or 3 instances of R³.
Embodiment 262. The compound of embodiment 193, or a pharmaceutically acceptable salt thereof, wherein Ring B is 1H indazol-5-yl, substituted at available positions with 0, 1, 2 or 3 instances of R³.
Embodiment 263. The compound of embodiment 193, or a pharmaceutically acceptable salt thereof, wherein Ring B is 1H indazol-4-yl, substituted at available positions with 0, 1, 2 or 3 instances of R³.
Embodiment 264. The compound of embodiment 193, or a pharmaceutically acceptable salt thereof, wherein Ring B is 2H-indazolyl, substituted at available positions with 0, 1, 2 or 3 instances of R³.
Embodiment 265. The compound of embodiment 193, or a pharmaceutically acceptable salt thereof, wherein Ring B is 2H-indazol-6-yl.
Embodiment 266. The compound of embodiment 193, or a pharmaceutically acceptable salt thereof, wherein Ring B is 2H-indazol-5-yl, substituted at available positions with 0, 1, 2 or 3 instances of R³.
Embodiment 267. The compound of embodiment 193, or a pharmaceutically acceptable salt thereof, wherein Ring B is quinolinyl, substituted at available positions with 0, 1, 2 or 3 instances of R³.
Embodiment 268. The compound of embodiment 193, or a pharmaceutically acceptable salt thereof, wherein Ring B is quinolin-6-yl, substituted at available positions with 0, 1, 2 or 3 instances of R³
Embodiment 269. The compound of embodiment 193, or a pharmaceutically acceptable salt thereof, wherein Ring B is quinolin-7-yl, substituted at available positions with 0, 1, 2 or 3 instances of R³.
Embodiment 270. The compound of embodiment 193, or a pharmaceutically acceptable salt thereof, wherein Ring B is isoquinolinyl, substituted at available positions with 0, 1, 2 or 3 instances of R³.
Embodiment 271. The compound of embodiment 193, or a pharmaceutically acceptable salt thereof, wherein Ring B is isoquinolin-6-yl, substituted at available positions with 0, 1, 2 or 3 instances of R³.
Embodiment 272. The compound of embodiment 193, or a pharmaceutically acceptable salt thereof, wherein Ring B is and benzo[d]thiazolyl, substituted at available positions with 0, 1, 2 or 3 instances of R³.
Embodiment 273. The compound of embodiment 193, or a pharmaceutically acceptable salt thereof, wherein Ring B is and benzo[d]thiazol-5-yl, substituted at available positions with 0, 1, 2 or 3 instances of R³.
Embodiment 274. The compound of embodiment 193, or a pharmaceutically acceptable salt thereof, wherein Ring B is selected from the group consisting of:

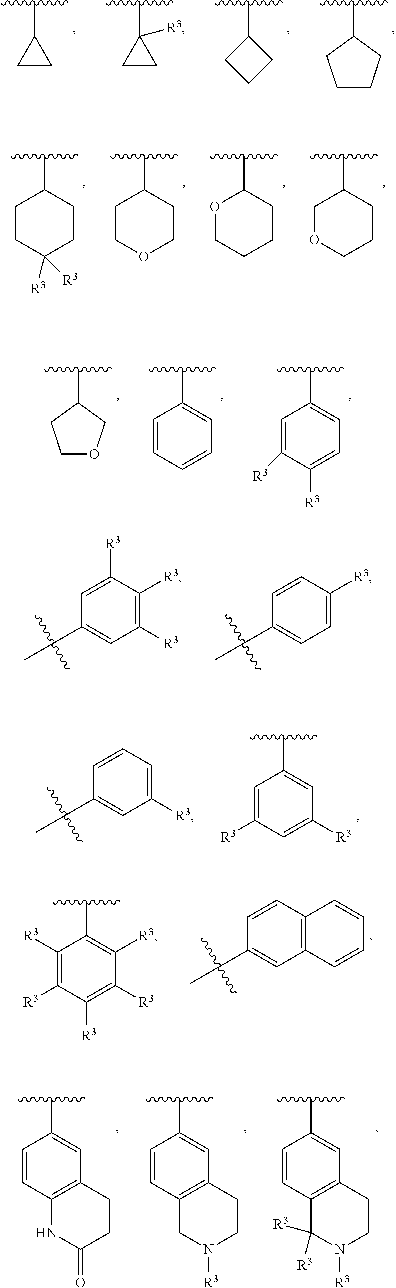

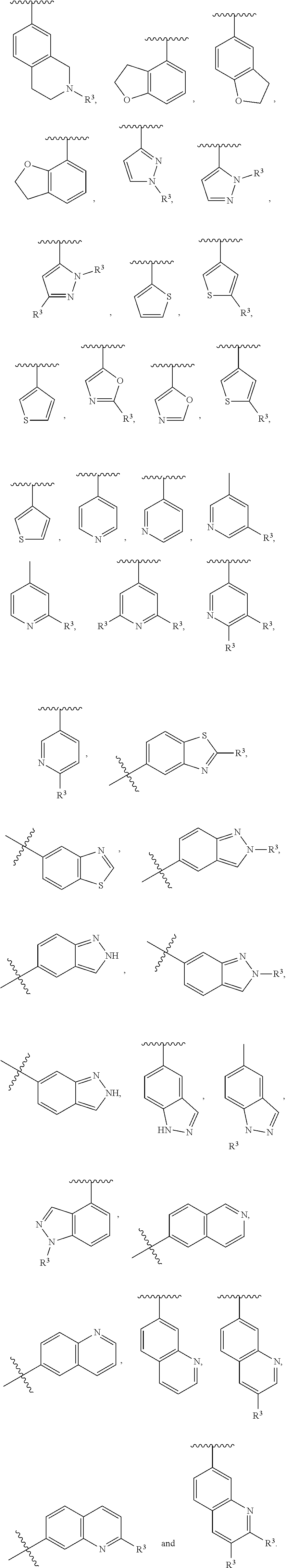

Embodiment 275. The compound of embodiment 193, or a pharmaceutically acceptable salt thereof, wherein Ring B is selected from the group consisting of:

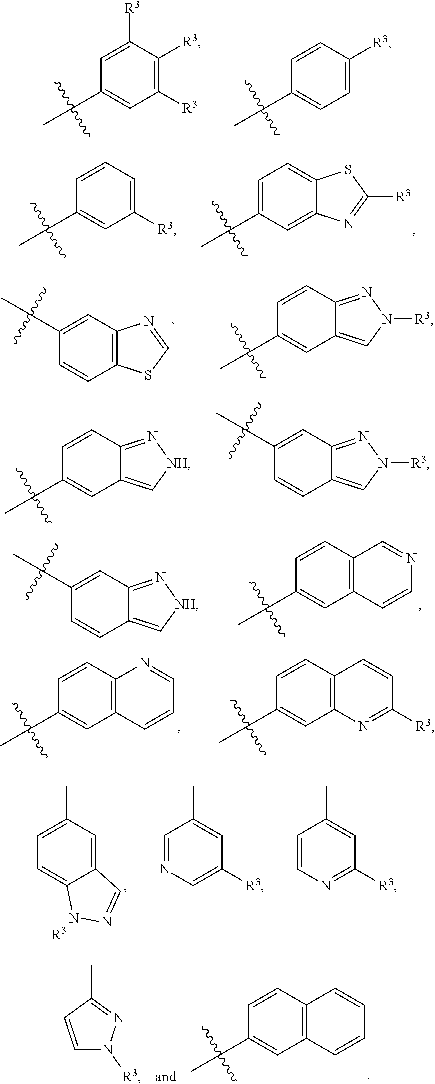

Embodiment 276. The compound of embodiment 193, or a pharmaceutically acceptable salt thereof, wherein Ring B is selected from the group consisting of:

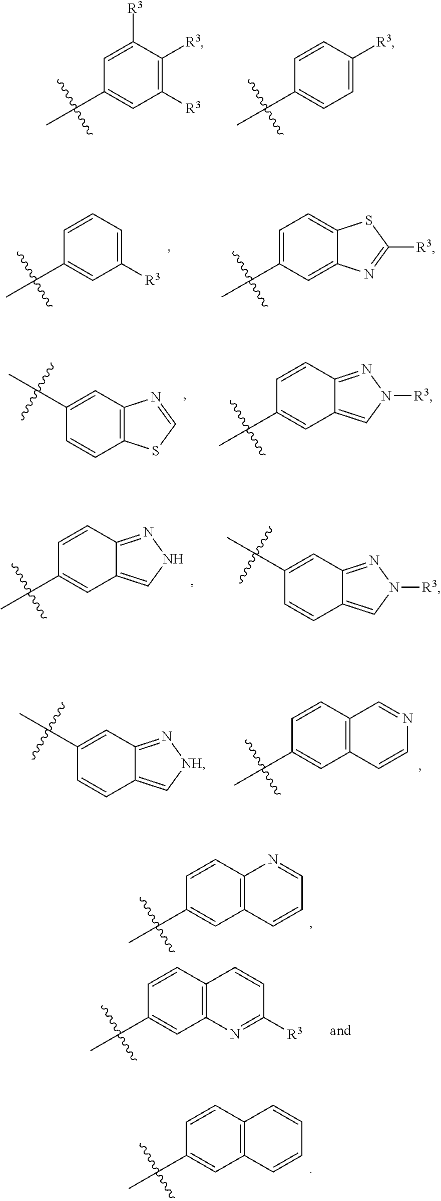

Embodiment 277. The compound of embodiment 193, or a pharmaceutically acceptable salt thereof, wherein Ring B is selected from the group consisting of:

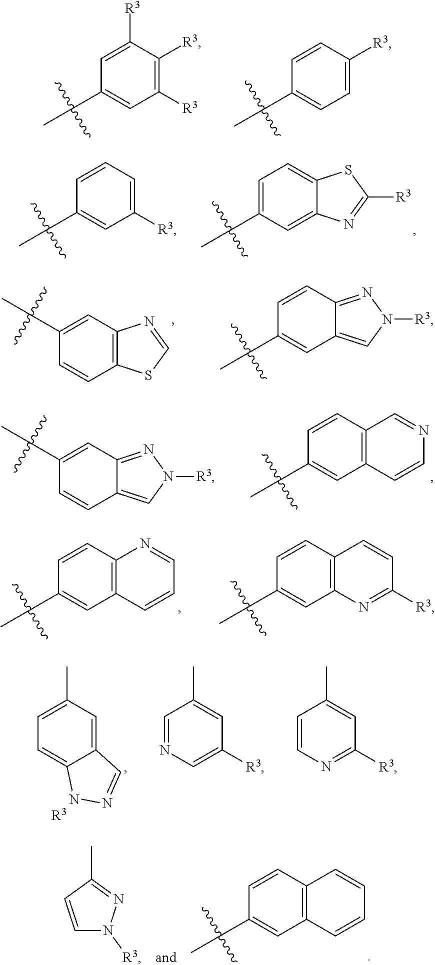

Embodiment 278. The compound of embodiment 193, or a pharmaceutically acceptable salt thereof, wherein Ring B is selected from the group consisting of:

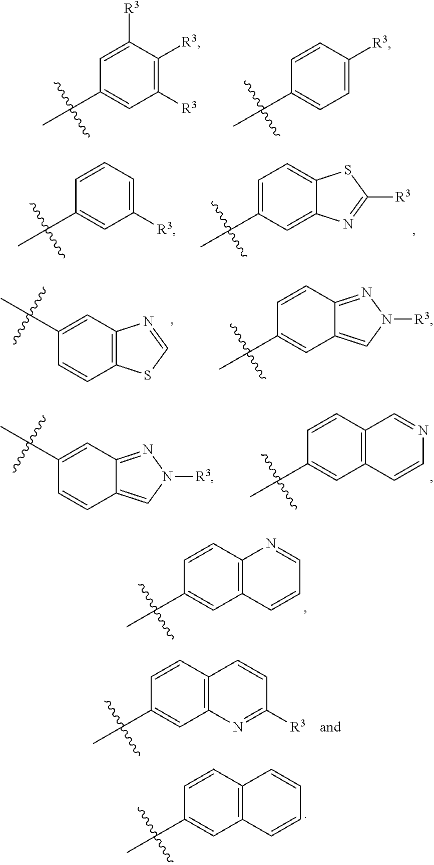

Embodiment 279. The compound of embodiment 193, or a pharmaceutically acceptable salt thereof, wherein Ring B is selected from the group consisting of:

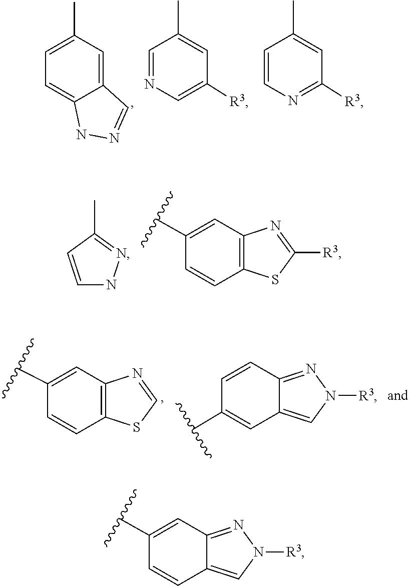

Embodiment 280. The compound of embodiment 193, or a pharmaceutically acceptable salt thereof, wherein Ring B is selected from the group consisting of:

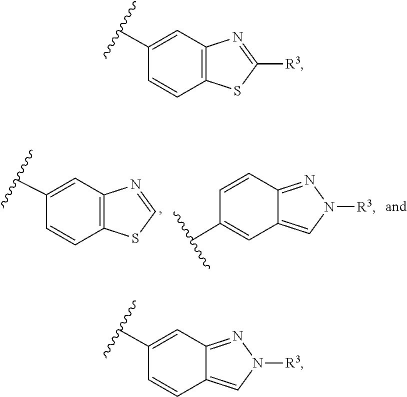

Embodiment 281. The compound of embodiment 193, or a pharmaceutically acceptable salt thereof, wherein Ring B is selected from the group consisting of:

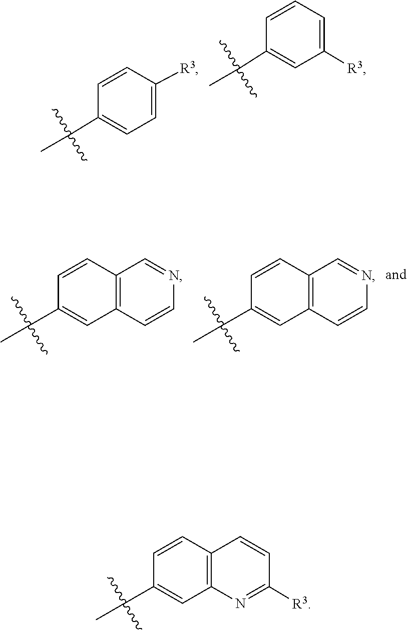

Embodiment 282. The compound of embodiment 193, or a pharmaceutically acceptable salt thereof, wherein Ring B is selected from the group consisting of:

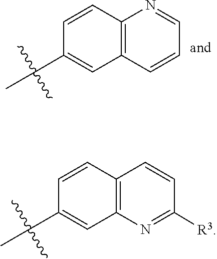

Embodiment 283. The compound of embodiment 193, or a pharmaceutically acceptable salt thereof, wherein Ring B is selected from the group consisting of:

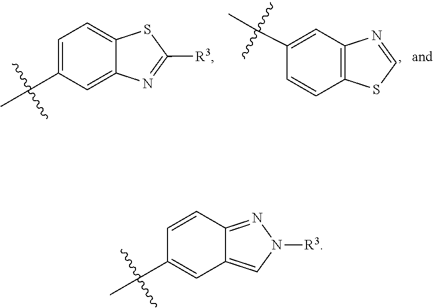

Embodiment 284. The compound of embodiment 193, or a pharmaceutically acceptable salt thereof, wherein Ring B is

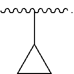 [SMILES: *C1CC1]

Embodiment 285. The compound of embodiment 193, or a pharmaceutically acceptable salt thereof, wherein Ring B is

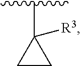

Embodiment 286. The compound of embodiment 193, or a pharmaceutically acceptable salt thereof, wherein Ring B is

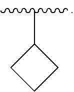 [SMILES: *C1CCC1]

Embodiment 287. The compound of embodiment 193, or a pharmaceutically acceptable salt thereof, wherein Ring B is

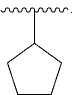 [SMILES: *C1CCCC1]

Embodiment 288. The compound of embodiment 193, or a pharmaceutically acceptable salt thereof, wherein Ring B is

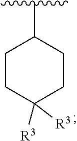

Embodiment 289. The compound of embodiment 193, or a pharmaceutically acceptable salt thereof, wherein Ring B is

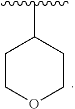 [SMILES: *C1CCOCC1]

Embodiment 290. The compound of embodiment 193, or a pharmaceutically acceptable salt thereof, wherein Ring B is

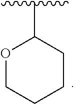 [SMILES: *C1CCCCO1]

Embodiment 291. The compound of embodiment 193, or a pharmaceutically acceptable salt thereof, wherein Ring B is

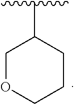 [SMILES: *C1CCCOC1]

Embodiment 292. The compound of embodiment 193, or a pharmaceutically acceptable salt thereof, wherein Ring B is

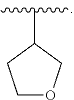 [SMILES: *C1CCOC1]

Embodiment 293. The compound of embodiment 193, or a pharmaceutically acceptable salt thereof, wherein Ring B is

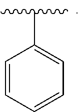 [SMILES: *c1ccccc1]

Embodiment 294. The compound of embodiment 193, or a pharmaceutically acceptable salt thereof, wherein Ring B is

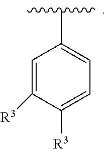

Embodiment 295. The compound of embodiment 193, or a pharmaceutically acceptable salt thereof, wherein Ring B is

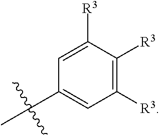

Embodiment 296. The compound of embodiment 193, or a pharmaceutically acceptable salt thereof, wherein Ring B is

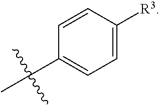

Embodiment 297. The compound of embodiment 193, or a pharmaceutically acceptable salt thereof, wherein Ring B is

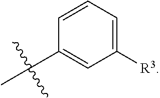

Embodiment 298. The compound of embodiment 193, or a pharmaceutically acceptable salt thereof, wherein Ring B is

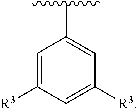

Embodiment 299. The compound of embodiment 193, or a pharmaceutically acceptable salt thereof, wherein Ring B is

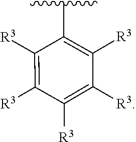

Embodiment 300. The compound of embodiment 193, or a pharmaceutically acceptable salt thereof, wherein Ring B is

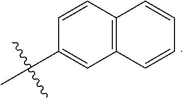 [SMILES: *c1ccc2ccccc2c1]

Embodiment 301. The compound of embodiment 193, or a pharmaceutically acceptable salt thereof, wherein Ring B is

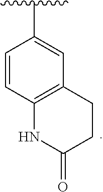 [SMILES: *c1ccc2c(c1)CCC(=O)N2]

Embodiment 302. The compound of embodiment 193, or a pharmaceutically acceptable salt thereof, wherein Ring B is

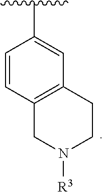

Embodiment 303. The compound of embodiment 193, or a pharmaceutically acceptable salt thereof, wherein Ring B is

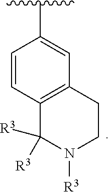

Embodiment 304. The compound of embodiment 193, or a pharmaceutically acceptable salt thereof, wherein Ring B is

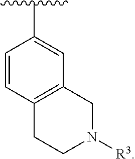

Embodiment 305. The compound of embodiment 193, or a pharmaceutically acceptable salt thereof, wherein Ring B is

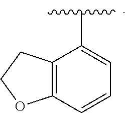 [SMILES: *c1cccc2c1CCO2]

Embodiment 306. The compound of embodiment 193, or a pharmaceutically acceptable salt thereof, wherein Ring B is

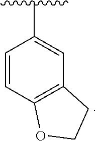 [SMILES: *c1ccc2c(c1)CCO2]

Embodiment 307. The compound of embodiment 193, or a pharmaceutically acceptable salt thereof, wherein Ring B is

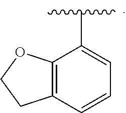 [SMILES: *c1cccc2c1OCC2]

Embodiment 308. The compound of embodiment 193, or a pharmaceutically acceptable salt thereof, wherein Ring B is

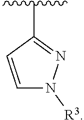

Embodiment 309. The compound of embodiment 193, or a pharmaceutically acceptable salt thereof, wherein Ring B is

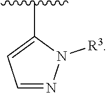

Embodiment 310. The compound of embodiment 193, or a pharmaceutically acceptable salt thereof, wherein Ring B is

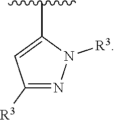

Embodiment 311. The compound of embodiment 193, or a pharmaceutically acceptable salt thereof, wherein Ring B is

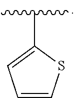 [SMILES: *c1cccs1]

Embodiment 312. The compound of embodiment 193, or a pharmaceutically acceptable salt thereof, wherein Ring B is.
Embodiment 313. The compound of embodiment 193, or a pharmaceutically acceptable salt thereof, wherein Ring B is

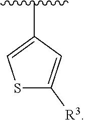

Embodiment 314. The compound of embodiment 193, or a pharmaceutically acceptable salt thereof, wherein Ring B is

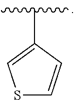 [SMILES: *c1ccsc1]

Embodiment 315. The compound of embodiment 193, or a pharmaceutically acceptable salt thereof, wherein Ring B is

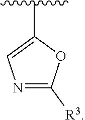

Embodiment 316. The compound of embodiment 193, or a pharmaceutically acceptable salt thereof, wherein Ring B is

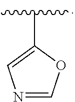 [SMILES: *c1cnco1]

Embodiment 317. The compound of embodiment 193, or a pharmaceutically acceptable salt thereof, wherein Ring B is

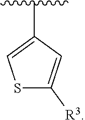

Embodiment 318. The compound of embodiment 193, or a pharmaceutically acceptable salt thereof, wherein Ring B is

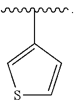 [SMILES: *c1ccsc1]

Embodiment 319. The compound of embodiment 193, or a pharmaceutically acceptable salt thereof, wherein Ring B is

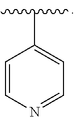 [SMILES: *c1ccncc1]

Embodiment 320. The compound of embodiment 193, or a pharmaceutically acceptable salt thereof, wherein Ring B is

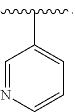 [SMILES: *c1cccnc1]

Embodiment 321. The compound of embodiment 193, or a pharmaceutically acceptable salt thereof, wherein Ring B is

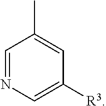

Embodiment 322. The compound of embodiment 193, or a pharmaceutically acceptable salt thereof, wherein Ring B is

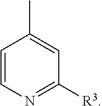

Embodiment 323. The compound of embodiment 193, or a pharmaceutically acceptable salt thereof, wherein Ring B is

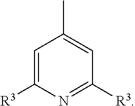

Embodiment 324. The compound of embodiment 193, or a pharmaceutically acceptable salt thereof, wherein Ring B is

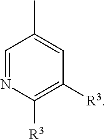

Embodiment 325. The compound of embodiment 193, or a pharmaceutically acceptable salt thereof, wherein Ring B is

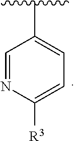

Embodiment 326. The compound of embodiment 193, or a pharmaceutically acceptable salt thereof, wherein Ring B is

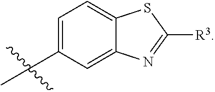

Embodiment 327. The compound of embodiment 193, or a pharmaceutically acceptable salt thereof, wherein Ring B is

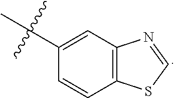 [SMILES: *c1ccc2scnc2c1]

Embodiment 328. The compound of embodiment 193, or a pharmaceutically acceptable salt thereof, wherein Ring B is

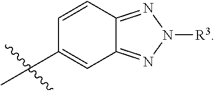

Embodiment 329. The compound of embodiment 193, or a pharmaceutically acceptable salt thereof, wherein Ring B is

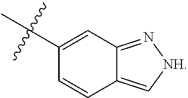 [SMILES: *c1ccc2c[nH]nc2c1]

Embodiment 330. The compound of embodiment 193, or a pharmaceutically acceptable salt thereof, wherein Ring B is

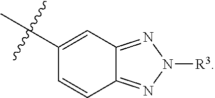

Embodiment 331. The compound of embodiment 193, or a pharmaceutically acceptable salt thereof, wherein Ring B is

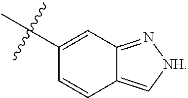 [SMILES: *c1ccc2c[nH]nc2c1]

Embodiment 332. The compound of embodiment 193, or a pharmaceutically acceptable salt thereof, wherein Ring B is

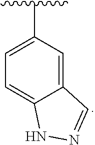 [SMILES: *c1ccc2[nH]ncc2c1]

Embodiment 333. The compound of embodiment 193, or a pharmaceutically acceptable salt thereof, wherein Ring B is

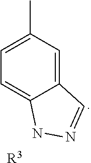

Embodiment 334. The compound of embodiment 193, or a pharmaceutically acceptable salt thereof, wherein Ring B is

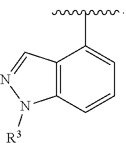

Embodiment 335. The compound of embodiment 193, or a pharmaceutically acceptable salt thereof, wherein Ring B is

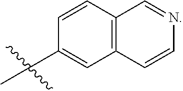 [SMILES: *c1ccc2cnccc2c1]

Embodiment 336. The compound of embodiment 193, or a pharmaceutically acceptable salt thereof, wherein Ring B is

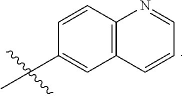 [SMILES: *c1ccc2ncccc2c1]

Embodiment 337. The compound of embodiment 193, or a pharmaceutically acceptable salt thereof, wherein Ring B is

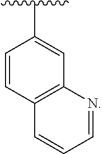 [SMILES: *c1ccc2cccnc2c1]

Embodiment 338. The compound of embodiment 193, or a pharmaceutically acceptable salt thereof, wherein Ring B is

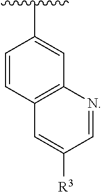

Embodiment 339. The compound of embodiment 193, or a pharmaceutically acceptable salt thereof, wherein Ring B is

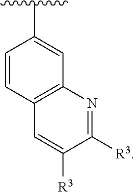

Embodiment 340. The compound of embodiment 193, or a pharmaceutically acceptable salt thereof, wherein Ring B is

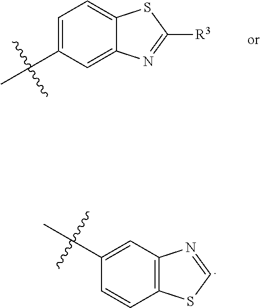

Embodiment 341. The compound of embodiment 193, or a pharmaceutically acceptable salt thereof, wherein Ring B is

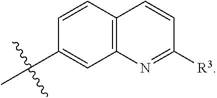

Embodiment 342. The compound of any one of embodiments 193 to 341, or a pharmaceutically acceptable salt thereof, wherein each R³ is independently selected from the group consisting of -D, —O, halo, —CN, —C₁-C₆ alkyl, —C₁-C₆ heteroalkyl, —C₁-C₆ haloalkyl, —C₃-C₉ cycloalkyl, 3-10 membered heterocyclyl, cycloalkylalkyl, heterocyclylalkyl, —OR^{a3}, —N(R^{a3})₂, —NR^{a3}C(═O)R^{a3}, wherein each alkyl, cycloalkyl, heterocyclyl, cycloalkylalkyl and heterocyclylalkyl is optionally substituted (e.g., with 0, 1, 2, 3, 4, or 5 instances of R⁶; and each R^{a3} is independently selected from the group consisting of H, —C₁-C₆ alkyl, —C₁-C₆ haloalkyl, heterocyclylalkyl substituted with 0 or 1 instances of -Me, —C₁-C₆ heteroalkyl substituted with 0 or 1 instances of ═O, C₃-C₉ cycloalkyl, and 3-10 membered heterocyclyl substituted with 0 or 1 instances of ═O, -Me or a combination thereof.
Embodiment 343. The compound of any one of embodiments 193 to 341, or a pharmaceutically acceptable salt thereof, wherein each R³ is independently selected from the group consisting of D, —CN, halo (e.g., —F, —Cl, Br), —C₁-C₆ alkyl (e.g., -Me, -Et, -Pr, -ⁱPr, -sec-Bu, -^{t}Bu, —CH₂(CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂, —C₁-C₆ haloalkyl, (e.g., —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OR^{a3} (e.g., —OH, —OCH₃, —OCH₂CH₃, —OCH₂CH₂N(CH₃)₂, —OCH₂CH(CH₃)N(CH₃)₂, —OCF₃, —OCHF₂, —O-piperidinyl, —OCH₂-pyrrolidinyl), —NR^{a3})₂, —NR^{a3}C(═O)R^{a3} (e.g., —NHC(═O)CH₃), —NHC(═O)CH₂CH₂N(CH₃)₂), wherein each alkyl, cycloalkyl, heterocyclyl, cycloalkylalkyl, and heterocyclylalkyl is optionally substituted (e.g., with 0, 1, 2, 3, 4, or 5 instances of R⁶; and each R^{a3} is independently selected from the group consisting of H, —C₁-C₆ alkyl, (e.g., -Me, -Et, -Pr, -ⁱPr, -sec-Bu, -^{t}Bu), —C₁-C₆ haloalkyl (e.g., —CF₃, —CHF₂, —CH₂CF₃), —C₁-C₆ heteroalkyl substituted with 0 or 1 instances of ═O (e.g., —CH₂CH₂N(CH₃)₂, —CH₂CH(CH₃)N(CH₃)₂, CH₂C(═O)N(CH₃)₂, —CH(CH₃)CH₂N(CH₃)₂, —CH(CH₃)C(═O)N(CH₃)₂)), C₃-C₉ cycloalkyl (e.g., cyclopropyl, cyclobutyl, cyclopentyl, cyclohexyl), heterocyclylalkyl (e.g., —CH₂(N-methyl pyrrolidin-2-yl)) and 3-10 membered heterocyclyl (e.g., piperidinyl, e.g., N-methyl piperidin-4-yl).
Embodiment 344. The compound of any one of embodiments 193 to 341, or a pharmaceutically acceptable salt thereof, wherein each R³ is independently selected from the group consisting of —CN, halo (e.g., —F, —Cl, Br), —C₁-C₆ alkyl (e.g., -Me, -Et, -Pr, -ⁱPr, -sec-Bu, -^{t}Bu, —CH₂(CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂)—C₁-C₆ haloalkyl, (e.g., —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OR^{a3} (e.g., —OH, —OCH₃, —OCH₂CH₃, —OCH₂CH₂N(CH₃)₂, —OCH₂CH(CH₃)N(CH₃)₂, —OCF₃, —OCHF₂, —O-piperidinyl, —OCH₂-pyrrolidinyl), —NR^{a3}C(═O)R^{a3} (e.g., —NHC(═O)CH₃), —NHC(═O)CH₂CH₂N(CH₃)₂), wherein each alkyl, cycloalkyl, heterocyclyl, cycloalkylalkyl, and heterocyclylalkyl is optionally substituted (e.g., with 0, 1, 2, 3, 4, or 5 instances of R⁶; and
each R^{a3} is independently selected from the group consisting of H, —C₁-C₆ alkyl, (e.g., -Me, -Et, -Pr, -ⁱPr, -sec-Bu, -^{t}Bu), —C₁-C₆ haloalkyl (e.g., —CF₃, —CHF₂, —CH₂CF₃), —C₁-C₆ heteroalkyl substituted with 0 or 1 instances of ═O (e.g., —CH₂CH₂N(CH₃)₂, —CH₂CH(CH₃)N(CH₃)₂, —CH₂C(═O)N(CH₃)₂, —CH(CH₃)CH₂N(CH₃)₂, CH(CH₃)C(═O)N(CH₃)₂)), heterocyclylalkyl (e.g., —CH₂(N-methyl pyrrolidin-2-yl)) and 3-10 membered heterocyclyl (e.g., piperidinyl, e.g., N-methyl piperidin-4-yl).
Embodiment 345. The compound of any one of embodiments 193 to 344, or a pharmaceutically acceptable salt thereof, wherein each R^{a3} is independently selected from the group consisting of H, -Me, -Et, -Pr, -ⁱPr, -sec-Bu, -^{t}Bu, —CF₃, —CHF₂, —CH₂CF₃, —CH₂(N-methyl pyrrolidin-2-yl), N-methyl piperidin-4-yl, —CH₂CH(CH₃)N(CH₃)₂, cyclopropyl and —CH₂CH₂N(CH₃)₂.
Embodiment 346. The compound of any one of embodiments 193 to 344, or a pharmaceutically acceptable salt thereof, wherein each R^{a3} is independently selected from the group consisting of H, -Me, -Et, -Pr, -ⁱPr, -sec-Bu, -^{t}Bu, —CF₃, —CHF₂, —CH₂CF₃, —CH₂(N-methyl pyrrolidin-2-yl), N-methyl piperidin-4-yl, —CH₂CH(CH₃)N(CH₃)₂, and —CH₂CH₂N(CH₃)₂.
Embodiment 347. The compound of any one of embodiments 193 to 344, or a pharmaceutically acceptable salt thereof, wherein R^{a3} is selected from -Me, -Et, -cyclopropyl, —CH₂CH₂N(CH₃)₂.
Embodiment 348. The compound of any one of embodiments 193 to 344, or a pharmaceutically acceptable salt thereof, wherein R^{a3} is —CH₂CH₂N(CH₃)₂.
Embodiment 349. The compound of any one of embodiments 193 to 341, or a pharmaceutically acceptable salt thereof, wherein each R³ is independently selected from the group consisting of D, —CN, -Me, -Et, -Pr, —F, —Cl, —CF₃, —N(CH₃)₂, —N(CH₂CH₃)₂, —N(CH₃)CH₂CH₃, —OMe, -OEt, —O-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, cyclohexyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 2-azabicyclo[2.2.1]heptanyl, 1-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —OCH₂CH₂N(CH₃)₂, —OCH₂CH(CH₃)N(CH₃)₂, —OCH₂(pyrrolidinyl), —Opiperidinyl, and —NHC(═O)CH₂CH₂N(CH₃)₂), wherein each cyclopropyl, cyclobutyl, cyclohexyl, tetrahydrofuranyl, tetrahydropyranyl, oxetanyl, morpholinyl, pyrrolidinyl, piperidinyl, tetrahydropyridinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, 2-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —CH(CH₃)CH₂-pyrrolidinyl and —(CH₂)₃-pyrrolidinyl is optionally substituted (e.g., with 0, 1, 2, 3, 4, or 5 instances of R⁶).
Embodiment 350. The compound of any one of embodiments 193 to 341, or a pharmaceutically acceptable salt thereof, wherein each R³ is independently selected from the group consisting of —CN, -Me, -Et, -Pr, —F, —Cl, —CF₃, —N(CH₃)₂, —N(CH₃)CH₂CH₃, —OMe, —OEt, —OCH₂-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —OCH₂CH₂N(CH₃)₂, —OCH₂CH(CH₃)N(CH₃)₂, —OCH₂(pyrrolidinyl), —Opiperidinyl, and —NHC(═O)CH₂CH₂N(CH₃)₂), wherein each cyclopropyl, cyclobutyl, tetrahydropyranyl, oxetanyl, morpholinyl, pyrrolidinyl, piperidinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —(CH₂)₂-pyrrolidinyl, —CH(CH₃)CH₂-pyrrolidinyl and —(CH₂)₃-pyrrolidinyl is optionally substituted (e.g., with 0, 1, 2, 3, 4, or 5 instances of R⁶).
Embodiment 351. The compound of any one of embodiments 193 to 341, or a pharmaceutically acceptable salt thereof, wherein each R³ is independently selected from the group consisting of —CN, -Me, —F, —Cl, —CF₃, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl, —OCH₂CH₂N(CH₃)₂, —OCH₂CH(CH₃)N(CH₃)₂, —OCH₂(pyrrolidinyl), —Opiperidinyl, and —NHC(═O)CH₂CH₂N(CH₃)₂), wherein each cyclopropyl, cyclobutyl, tetrahydropyranyl, oxetanyl, morpholinyl, pyrrolidinyl, piperidinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, —CH₂-piperidinyl, —CH₂-pyrrolidinyl, —(CH₂)₂-pyrrolidinyl, —CH(CH₃)CH₂-pyrrolidinyl and —(CH₂)₃-pyrrolidinyl is optionally substituted (e.g., with 0, 1, 2, 3, 4, or 5 instances of R⁶).
Embodiment 352. The compound of any one of embodiments 193 to 341, or a pharmaceutically acceptable salt thereof, wherein each R³ is independently selected from the group consisting of: D, —CN, -Me, -Et, -Pr, —F, —Cl, —CF₃, —N(CH₃)₂, —N(CH₃)CH₂CH₃, —OMe, —OEt, —OCH₂-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, cyclohexyl, tetrahydrofuran-3-yl, tetrahydropyran-4-yl, oxetan-3-yl, morpholin-2-yl, piperazin-4-yl, pyrrolidin-1-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 4-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl, —O(CH₂)₂N(CH₃)₂—OCH₂(pyrrolidin-2-yl), —Opiperidin-4-yl, —OCH₂CH(CH₃)N(CH₃)₂ and —NHC(═O)(CH₂)₂N(CH₃)₂, wherein each cyclopropyl, cyclobutyl, cyclohexyl, tetrahydrofuran-3-yl, tetrahydropyran-4-yl, oxetan-3-yl, morpholin-2-yl, piperazin-4-yl, pyrrolidin-1-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 4-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl and —CH(CH₃)CH₂-pyrrolidin-1-yl is optionally substituted (e.g., with 0, 1, 2, 3, 4, or 5 instances of R⁶).
Embodiment 353. The compound of any one of embodiments 193 to 341, or a pharmaceutically acceptable salt thereof, wherein each R³ is independently selected from the group consisting of:—CN, -Me, -Et, -Pr, —F, —Cl, —CF₃, —N(CH₃)₂, —N(CH₃)CH₂CH₃, —OMe, —OEt, —OCH₂-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuran-3-yl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl, —O(CH₂)₂N(CH₃)₂—OCH₂(pyrrolidin-2-yl), —Opiperidin-4-yl, —OCH₂CH(CH₃)N(CH₃)₂ and —NHC(═O)(CH₂)₂N(CH₃)₂, wherein each cyclopropyl, cyclobutyl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, piperazin-4-yl, azetidin-3-yl, 2-azabicyclo[2.2.2]octan-4-yl, 2-azaspiro[3.3]heptan-6-yl, 1-azabicyclo[2.2.1]heptan-4-yl, —CH₂-piperidin-4-yl, —CH₂-pyrrolidin-1-yl, —(CH₂)₂-pyrrolidin-1-yl and —CH(CH₃)CH₂-pyrrolidin-1-yl is optionally substituted (e.g., with 0, 1, 2, 3, 4, or 5 instances of R⁶).
Embodiment 354. The compound of any one of embodiments 193 to 341, or a pharmaceutically acceptable salt thereof, wherein each R³ is independently selected from the group consisting of:—CN, -Me, —F, —Cl, —CF₃, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, tetrahydrofuran-3-yl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, 1,2,3,6 tetrahydropyridin-4-yl, azetidin-3-yl, 2-azabicyclo[2.2.2]octan-4-yl, 2-azaspiro[3.3]heptan-6-yl, 1-azabicyclo[2.2.1]heptan-4-yl, 1-azabicyclo[2.2.1]heptan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl, —O(CH₂)₂N(CH₃)₂—OCH₂(pyrrolidin-2-yl), —Opiperidin-4-yl, —OCH₂CH(CH₃)N(CH₃)₂ and —NHC(═O)(CH₂)₂N(CH₃)₂, wherein each cyclopropyl, cyclobutyl, tetrahydropyran-4-yl, oxetan-3-yl, morpholin-2-yl, piperazin-4-yl, pyrrolidin-3-yl, piperidin-4-yl, piperidin-3-yl, piperidin-2-one-4-yl, piperazin-4-yl, azetidin-3-yl, 2-azabicyclo[2.2.2]octan-4-yl, 2-azaspiro[3.3]heptan-6-yl, 1-azabicyclo[2.2.1]heptan-4-yl, —CH₂-piperidin-4-yl, —CH₂-pyrrolidin-1-yl, —(CH₂)₂-pyrrolidin-1-yl and —CH(CH₃)CH₂-pyrrolidin-1-yl is optionally substituted (e.g., with 0, 1, 2, 3, 4, or 5 instances of R⁶).
Embodiment 355. The compound of any one of embodiments 193 to 341, or a pharmaceutically acceptable salt thereof, wherein R³ is selected from the group consisting of 3-8 membered monocyclic heterocyclyl, and 5-10 membered bicyclic heterocyclyl, wherein each heterocyclyl is optionally substituted ((e.g., substituted with 0, 1, 2, 3, 4, or 5 instances of R⁶).
Embodiment 356. The compound of any one of embodiments 193 to 341, or a pharmaceutically acceptable salt thereof, wherein each R⁶ is independently selected from the group consisting of -Me, -Et, -ⁱPr, -CD₃, cyclopropyl, oxetan-3-yl, —OH, ═O, —F, —OCH₃, —CH₂CH₂F, —CH₂CHF₂, —CH₂CH₂CF₃, —C(═O)CH₃, —N(CH₃)₂, —CH₂N(CH₃)₂, —CH₂N(CH₃)CH₂CH₃, —N(ⁱPr) (CH₂CH₃), —N(ⁱPr) (CH₃), —N(CH₂CH₃)₂, —N(CH₃)(CH₂CH₃), —NHC(═O)CH₃).
Embodiment 357. The compound of any one of embodiments 193 to 341, or a pharmaceutically acceptable salt thereof, wherein each R⁶ is independently selected from the group consisting of -Me, -Et, -CD₃, cyclopropyl, oxetan-3-yl, —OH, ═O, —N(CH₃)₂ and —CH₂N(CH₃)₂.
Embodiment 358. The compound of any one of embodiments 193 to 341, or a pharmaceutically acceptable salt thereof, wherein R³ is selected from the group consisting of -D, —CN, -Me, -Et, -ⁱPr, —OMe, -OEt, —F, —Cl, —CF₃,

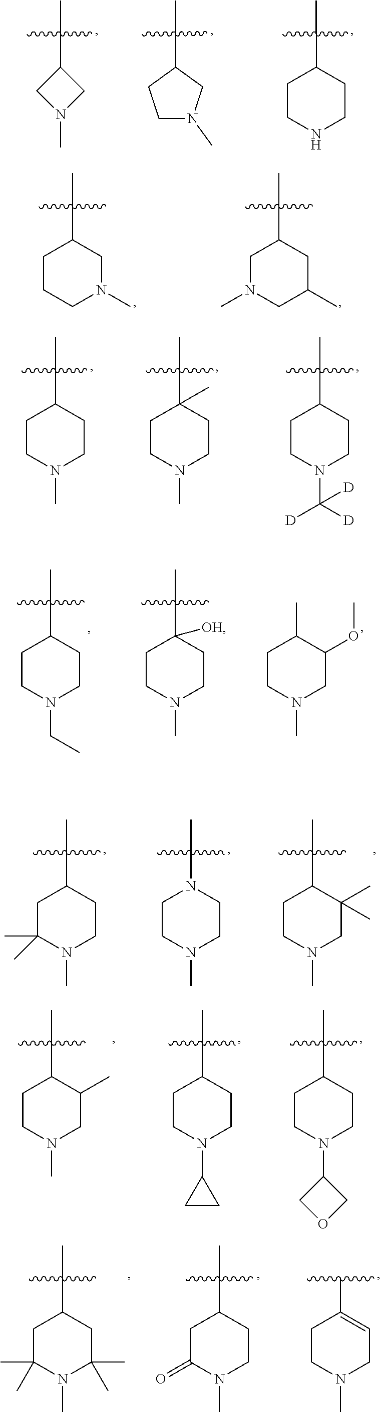

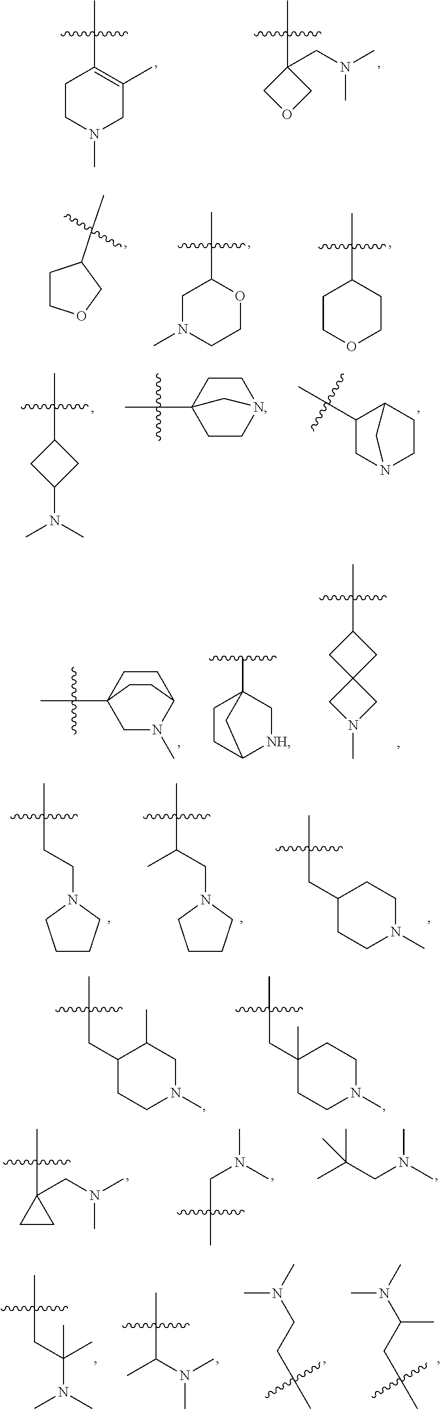

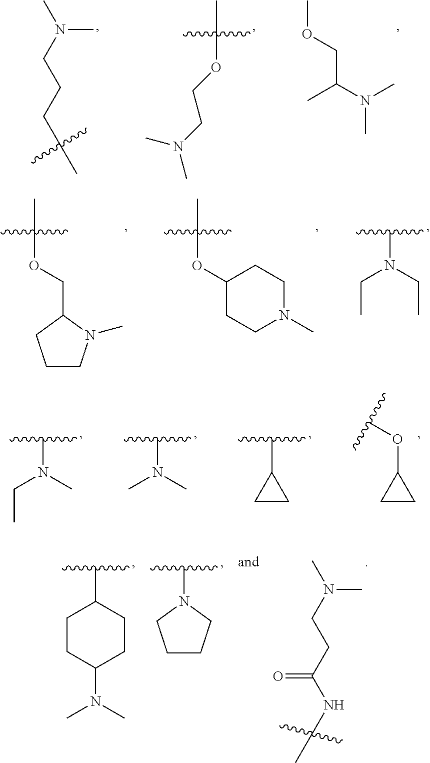

Embodiment 359. The compound of any one of embodiments 193 to 341, or a pharmaceutically acceptable salt thereof, wherein R³ is selected from the group consisting of -D, —CN, -Me, -Et, -ⁱPr, —OMe, -OEt, —F, —Cl, —CF₃,

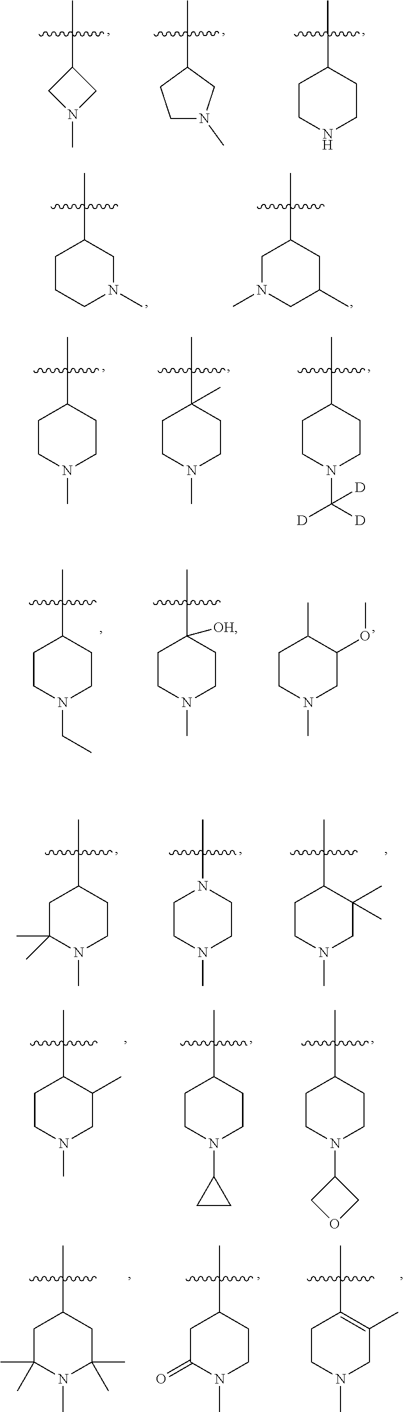

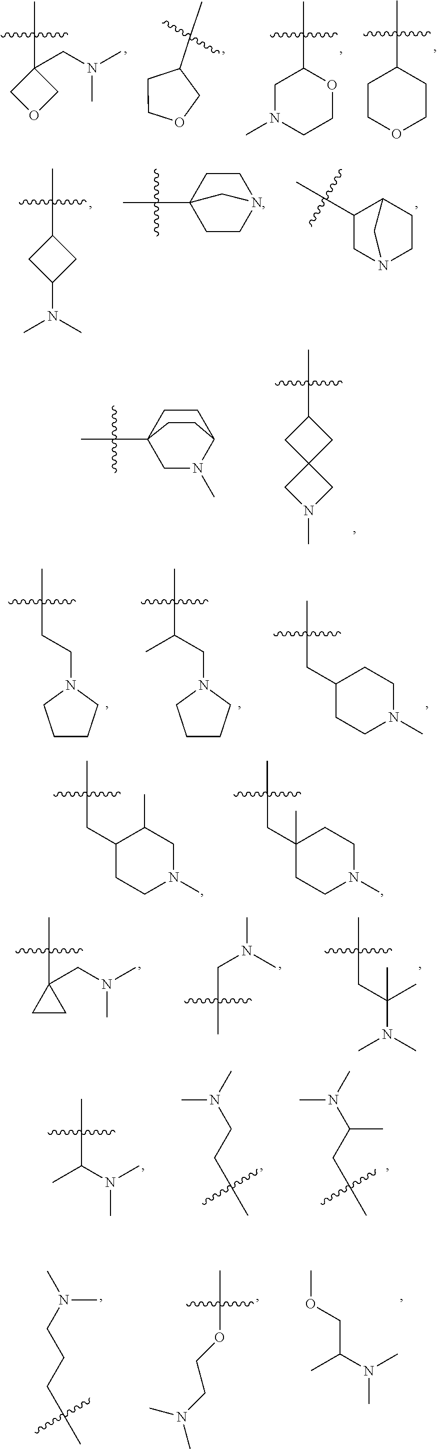

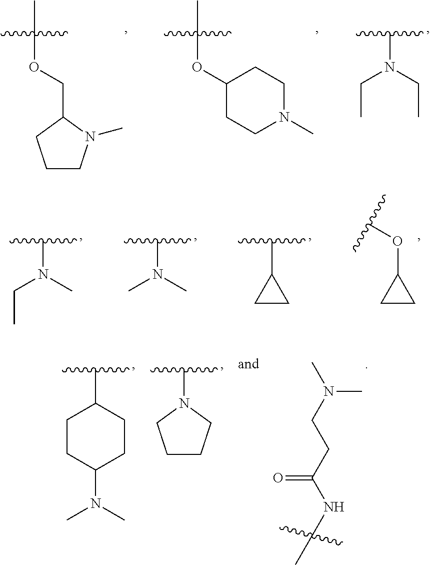

Embodiment 360. The compound of any one of embodiments 193 to 341, or a pharmaceutically acceptable salt thereof, wherein R³ is selected from the group consisting of -D, —CN, -Me, —F, —Cl, —CF₃,

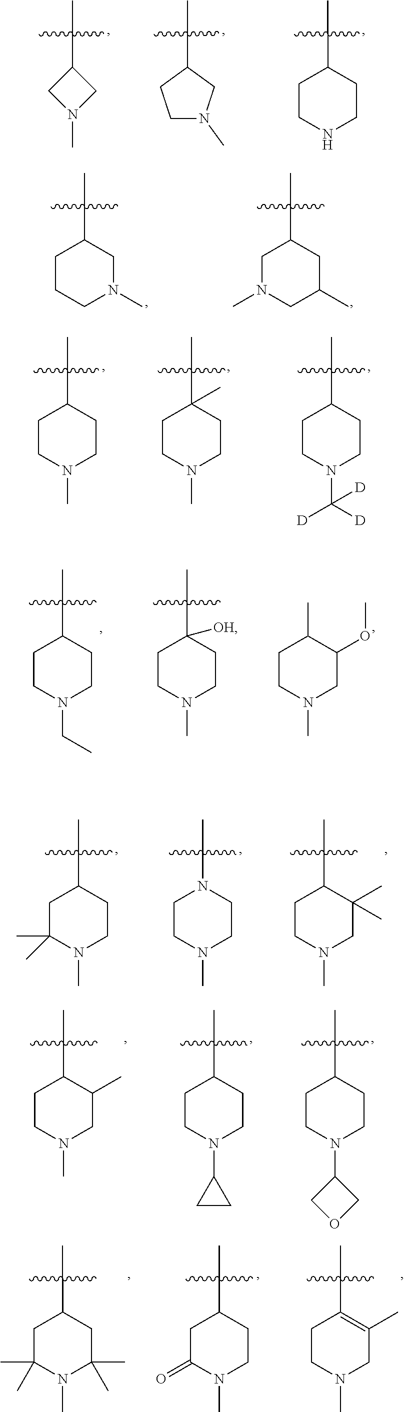

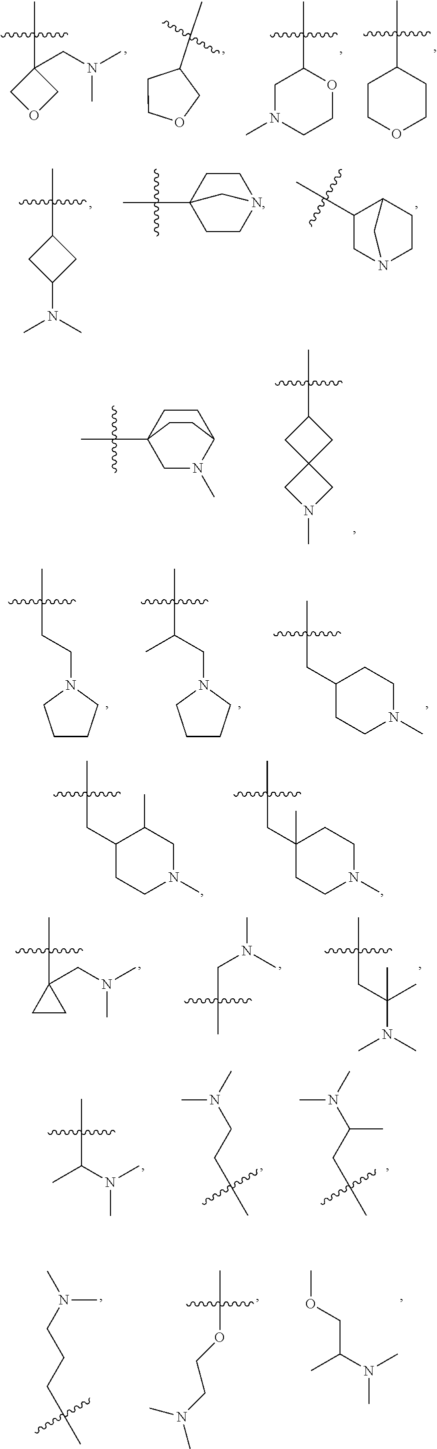

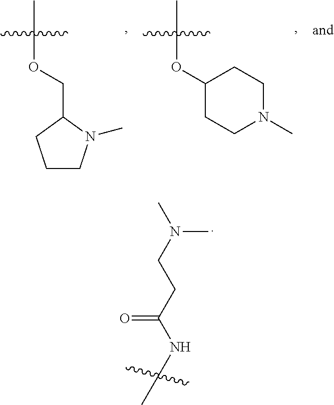

Embodiment 361. The compound of any one of embodiments 193 to 341, or a pharmaceutically acceptable salt thereof, wherein R³ is -D.
Embodiment 362. The compound of any one of embodiments 193 to 341, or a pharmaceutically acceptable salt thereof, wherein R³ is —CN.
Embodiment 363. The compound of any one of embodiments 193 to 341, or a pharmaceutically acceptable salt thereof, wherein R³ is -Me.
Embodiment 364. The compound of any one of embodiments 193 to 341, or a pharmaceutically acceptable salt thereof, wherein R³ is -Et.
Embodiment 365. The compound of any one of embodiments 193 to 341, or a pharmaceutically acceptable salt thereof, wherein R³ is -ⁱPr.
Embodiment 366. The compound of any one of embodiments 193 to 341, or a pharmaceutically acceptable salt thereof, wherein R³ is —OMe.
Embodiment 367. The compound of any one of embodiments 193 to 341, or a pharmaceutically acceptable salt thereof, wherein R³ is -OEt.
Embodiment 368. The compound of any one of embodiments 193 to 341, or a pharmaceutically acceptable salt thereof, wherein R³ is —F.
Embodiment 369. The compound of any one of embodiments 193 to 341, or a pharmaceutically acceptable salt thereof, wherein R³ is —Cl.
Embodiment 370. The compound of any one of embodiments 193 to 341, or a pharmaceutically acceptable salt thereof, wherein R³ is —CF₃.
Embodiment 371. The compound of any one of embodiments 193 to 341, or a pharmaceutically acceptable salt thereof, wherein R³ is

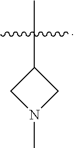 [SMILES: *C1CN(C)C1]

Embodiment 372. The compound of any one of embodiments 193 to 341, or a pharmaceutically acceptable salt thereof, wherein R³ is

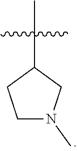 [SMILES: *C1CCN(C)C1]

Embodiment 373. The compound of any one of embodiments 193 to 341, or a pharmaceutically acceptable salt thereof, wherein R³ is

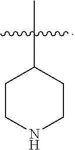 [SMILES: *C1CCNCC1]

Embodiment 374. The compound of any one of embodiments 193 to 341, or a pharmaceutically acceptable salt thereof, wherein R³ is

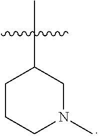 [SMILES: *C1CCCN(C)C1]

Embodiment 375. The compound of any one of embodiments 193 to 341, or a pharmaceutically acceptable salt thereof, wherein R³ is

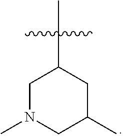

Embodiment 376. The compound of any one of embodiments 193 to 341, or a pharmaceutically acceptable salt thereof, wherein R³ is

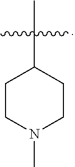 [SMILES: *C1CCN(C)CC1]

Embodiment 377. The compound of any one of embodiments 193 to 341, or a pharmaceutically acceptable salt thereof, wherein R³ is

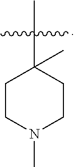 [SMILES: *C1(C)CCN(C)CC1]

Embodiment 378. The compound of any one of embodiments 193 to 341, or a pharmaceutically acceptable salt thereof, wherein R³ is

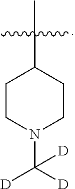 [SMILES: *C1CCN(C([2H])([2H])[2H])CC1]

Embodiment 379. The compound of any one of embodiments 193 to 341, or a pharmaceutically acceptable salt thereof, wherein R³ is

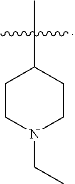 [SMILES: *C1CCN(CC)CC1]

Embodiment 380. The compound of any one of embodiments 193 to 341, or a pharmaceutically acceptable salt thereof, wherein R³ is

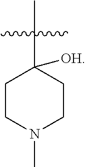 [SMILES: *C1(O)CCN(C)CC1]

Embodiment 381. The compound of any one of embodiments 193 to 341, or a pharmaceutically acceptable salt thereof, wherein R³ is

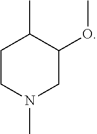

Embodiment 382. The compound of any one of embodiments 193 to 341, or a pharmaceutically acceptable salt thereof, wherein R³ is

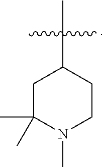 [SMILES: *C1CCN(C)C(C)(C)C1]

Embodiment 383. The compound of any one of embodiments 193 to 341, or a pharmaceutically acceptable salt thereof, wherein R³ is

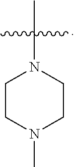 [SMILES: *N1CCN(C)CC1]

Embodiment 384. The compound of any one of embodiments 193 to 341, or a pharmaceutically acceptable salt thereof, wherein R³ is

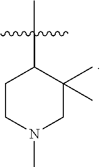 [SMILES: *C1CCN(C)CC1(C)C]

Embodiment 385. The compound of any one of embodiments 193 to 341, or a pharmaceutically acceptable salt thereof, wherein R³ is

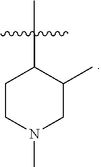 [SMILES: *C1CCN(C)CC1C]

Embodiment 386. The compound of any one of embodiments 193 to 341, or a pharmaceutically acceptable salt thereof, wherein R³ is

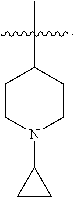 [SMILES: *C1CCN(C2CC2)CC1]

Embodiment 387. The compound of any one of embodiments 193 to 341, or a pharmaceutically acceptable salt thereof, wherein R³ is

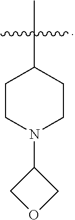 [SMILES: *C1CCN(C2COC2)CC1]

Embodiment 388. The compound of any one of embodiments 193 to 341, or a pharmaceutically acceptable salt thereof, wherein R³ is

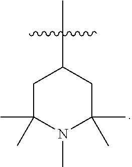

Embodiment 389. The compound of any one of embodiments 193 to 341, or a pharmaceutically acceptable salt thereof, wherein R³ is

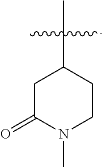 [SMILES: *C1CCN(C)C(=O)C1]

Embodiment 390. The compound of any one of embodiments 193 to 341, or a pharmaceutically acceptable salt thereof, wherein R³ is

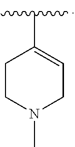 [SMILES: *C1=CCN(C)CC1]

Embodiment 391. The compound of any one of embodiments 193 to 341, or a pharmaceutically acceptable salt thereof, wherein R³ is

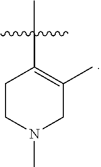 [SMILES: *C1=C(C)CN(C)CC1]

Embodiment 392. The compound of any one of embodiments 193 to 341, or a pharmaceutically acceptable salt thereof, wherein R³ is

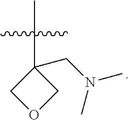 [SMILES: *C1(CN(C)C)COC1]

Embodiment 393. The compound of any one of embodiments 193 to 341, or a pharmaceutically acceptable salt thereof, wherein R³ is

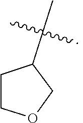 [SMILES: *C1CCOC1]

Embodiment 394. The compound of any one of embodiments 193 to 341, or a pharmaceutically acceptable salt thereof, wherein R³ is

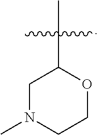 [SMILES: *C1CN(C)CCO1]

Embodiment 395. The compound of any one of embodiments 193 to 341, or a pharmaceutically acceptable salt thereof, wherein R³ is

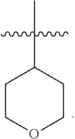 [SMILES: *C1CCOCC1]

Embodiment 396. The compound of any one of embodiments 193 to 341, or a pharmaceutically acceptable salt thereof, wherein R³ is

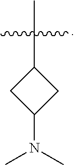

Embodiment 397. The compound of any one of embodiments 193 to 341, or a pharmaceutically acceptable salt thereof, wherein R³ is

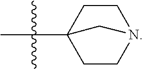 [SMILES: *C12CCN(CC1)C2]

Embodiment 398. The compound of any one of embodiments 193 to 341, or a pharmaceutically acceptable salt thereof, wherein R³ is

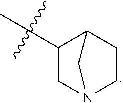 [SMILES: *C1CN2CCC1C2]

Embodiment 399. The compound of any one of embodiments 193 to 341, or a pharmaceutically acceptable salt thereof, wherein R³ is

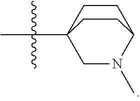 [SMILES: *C12CCC(CC1)N(C)C2]

Embodiment 400. The compound of any one of embodiments 193 to 341, or a pharmaceutically acceptable salt thereof, wherein R³ is

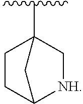 [SMILES: *C12CCC(C1)NC2]

Embodiment 401. The compound of any one of embodiments 193 to 341, or a pharmaceutically acceptable salt thereof, wherein R³ is

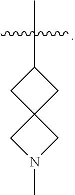 [SMILES: *C1CC2(C1)CN(C)C2]

Embodiment 402. The compound of any one of embodiments 193 to 341, or a pharmaceutically acceptable salt thereof, wherein R³ is

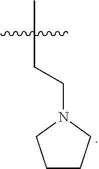 [SMILES: *CCN1CCCC1]

Embodiment 403. The compound of any one of embodiments 193 to 341, or a pharmaceutically acceptable salt thereof, wherein R³ is

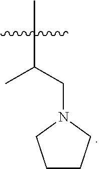 [SMILES: *C(C)CN1CCCC1]

Embodiment 404. The compound of any one of embodiments 193 to 341, or a pharmaceutically acceptable salt thereof, wherein R³ is

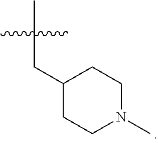 [SMILES: *CC1CCN(C)CC1]

Embodiment 405. The compound of any one of embodiments 193 to 341, or a pharmaceutically acceptable salt thereof, wherein R³ is

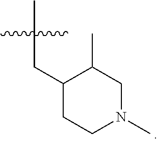 [SMILES: *CC1CCN(C)CC1C]

Embodiment 406. The compound of any one of embodiments 193 to 341, or a pharmaceutically acceptable salt thereof, wherein R³ is

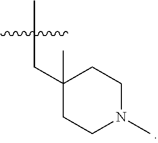 [SMILES: *CC1(C)CCN(C)CC1]

Embodiment 407. The compound of any one of embodiments 193 to 341, or a pharmaceutically acceptable salt thereof, wherein R³ is

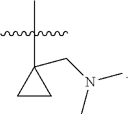 [SMILES: *C1(CN(C)C)CC1]

Embodiment 408. The compound of any one of embodiments 193 to 341, or a pharmaceutically acceptable salt thereof, wherein R³ is

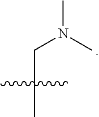 [SMILES: *CN(C)C]

Embodiment 409. The compound of any one of embodiments 193 to 341, or a pharmaceutically acceptable salt thereof, wherein R³ is

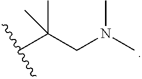 [SMILES: *C(C)(C)CN(C)C]

Embodiment 410. The compound of any one of embodiments 193 to 341, or a pharmaceutically acceptable salt thereof, wherein R³ is

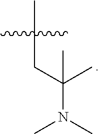 [SMILES: *CC(C)(C)N(C)C]

Embodiment 411. The compound of any one of embodiments 193 to 341, or a pharmaceutically acceptable salt thereof, wherein R³ is

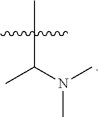 [SMILES: *C(C)N(C)C]

Embodiment 412. The compound of any one of embodiments 193 to 341, or a pharmaceutically acceptable salt thereof, wherein R³ is

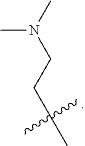

Embodiment 413. The compound of any one of embodiments 193 to 341, or a pharmaceutically acceptable salt thereof, wherein R³ is

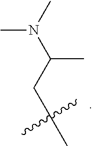 [SMILES: *CC(C)N(C)C]

Embodiment 414. The compound of any one of embodiments 193 to 341, or a pharmaceutically acceptable salt thereof, wherein R³ is

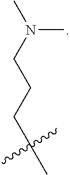 [SMILES: *CCCN(C)C]

Embodiment 415. The compound of any one of embodiments 193 to 341, or a pharmaceutically acceptable salt thereof, wherein R³ is

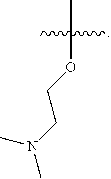 [SMILES: *OCCN(C)C]

Embodiment 416. The compound of any one of embodiments 193 to 341, or a pharmaceutically acceptable salt thereof, wherein R³ is

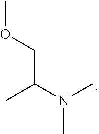

Embodiment 417. The compound of any one of embodiments 193 to 341, or a pharmaceutically acceptable salt thereof, wherein R³ is

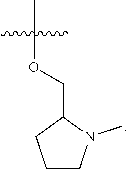 [SMILES: *OCC1CCCN1C]

Embodiment 418. The compound of any one of embodiments 193 to 341, or a pharmaceutically acceptable salt thereof, wherein R³ is

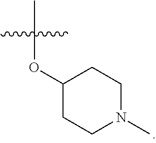 [SMILES: *OC1CCN(C)CC1]

Embodiment 419. The compound of any one of embodiments 193 to 341, or a pharmaceutically acceptable salt thereof, wherein R³ is

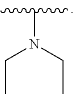 [SMILES: *N(CC)CC]

Embodiment 420. The compound of any one of embodiments 193 to 341, or a pharmaceutically acceptable salt thereof, wherein R³ is

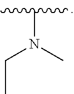 [SMILES: *N(C)CC]

Embodiment 421. The compound of any one of embodiments 193 to 341, or a pharmaceutically acceptable salt thereof, wherein R³ is

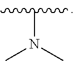

Embodiment 422. The compound of any one of embodiments 193 to 341, or a pharmaceutically acceptable salt thereof, wherein R³ is

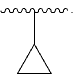 [SMILES: *C1CC1]

Embodiment 423. The compound of any one of embodiments 193 to 341, or a pharmaceutically acceptable salt thereof, wherein R³ is

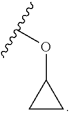 [SMILES: *OC1CC1]

Embodiment 424. The compound of any one of embodiments 193 to 341, or a pharmaceutically acceptable salt thereof, wherein R³ is

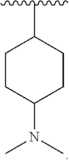 [SMILES: *C1CCC(N(C)C)CC1]

Embodiment 425. The compound of any one of embodiments 193 to 341, or a pharmaceutically acceptable salt thereof, wherein R³ is

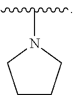 [SMILES: *N1CCCC1]

Embodiment 426. The compound of any one of embodiments 193 to 341, or a pharmaceutically acceptable salt thereof, wherein R³ is

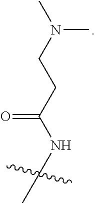 [SMILES: *NC(=O)CCN(C)C]

Embodiment 427. The compound of any one of embodiments 1 to 426, or a pharmaceutically acceptable salt thereof, wherein the compound is selected from Table 1, or a pharmaceutically acceptable salt thereof.
Embodiment 428. A pharmaceutical composition comprising a compound of any one of embodiments 1 to 427, or a pharmaceutically acceptable salt thereof, and a pharmaceutically acceptable carrier.
Embodiment 429. The pharmaceutical composition of embodiment 428, further comprising a second therapeutic agent.
Embodiment 430. A method of treating an MTAP-deficient and/or an MTA-accumulating disease in a subject in need thereof by administering to the subject an effective amount (e.g., a therapeutically effective amount) of a compound of any one of embodiments 1 to 427, or a pharmaceutically acceptable salt thereof, or a pharmaceutically acceptable composition of embodiment 428.
Embodiment 431. The method of embodiment 430, wherein the compound, or a pharmaceutically acceptable salt thereof, or composition is administered in combination with a second therapeutic agent.
Embodiment 432. A method of treating an MTAP-deficient and/or an MTA-accumulating disease in a subject in need thereof by administering to the subject an effective amount (e.g., a therapeutically effective amount) of a pharmaceutically acceptable composition of embodiment 429.
Embodiment 433. The method of any one of embodiments 430 to 432 wherein the disease is a proliferating disease.
Embodiment 434. The method of embodiment 433 wherein the disease is an MTAP-deficient and/or MTA-accumulating cancer.
Embodiment 435. The method of embodiment 434 wherein the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.
Embodiment 436. A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a compound of any one of embodiments 1 to 427, or a pharmaceutically acceptable salt thereof, or a pharmaceutically acceptable composition of embodiment 428 or 429 to the subject identified in step b).
Embodiment 437. Use of a compound of any one of embodiments 1 to 427, or a pharmaceutically acceptable salt thereof, or of a pharmaceutically acceptable composition of embodiment 428 for treating an MTAP-deficient and/or an MTA-accumulating disease in a subject in need thereof.
Embodiment 438. The use of embodiment 437 wherein the compound, or a pharmaceutically acceptable salt thereof, or composition is configured to be administered in combination with a second therapeutic agent.
Embodiment 439. Use of a pharmaceutically acceptable composition of embodiment 429 for treating an MTAP-deficient and/or an MTA-accumulating disease in a subject in need thereof.
Embodiment 440. The use of any one of embodiments 437 to 439 wherein the disease is a proliferating disease.
Embodiment 441. The use of embodiment 440 wherein the disease is an MTAP-deficient and/or MTA-accumulating cancer.
Embodiment 442. The use of embodiment 441 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.
Embodiment 443. A compound of any one of embodiments 1 to 427, or a pharmaceutically acceptable salt thereof, or a pharmaceutically acceptable composition of embodiment 428 for treating an MTAP-deficient and/or an MTA-accumulating disease in a subject in need thereof.
Embodiment 444. The compound for use of embodiment 443 wherein the compound, or a pharmaceutically acceptable salt thereof, or composition is configured to be administered in combination with a second therapeutic agent.
Embodiment 445. A pharmaceutically acceptable composition of embodiment 429 for use in treating an MTAP-deficient and/or an MTA-accumulating disease in a subject in need thereof.
Embodiment 446. The compound or composition for use of any one of embodiments 443 to 445 wherein the disease is a proliferating disease.
Embodiment 447. The compound or composition for use of embodiment 446 wherein the disease is an MTAP-deficient and/or MTA-accumulating cancer.
Embodiment 448. The compound or composition for use of embodiment 447 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.
Embodiment 449. Use of a compound of any one of embodiments 1 to 427, or a pharmaceutically acceptable salt thereof, or of a pharmaceutically acceptable composition of embodiment 428 in the manufacturing of a medicament for treating an MTAP-deficient and/or an MTA-accumulating disease in a subject in need thereof.
Embodiment 450. The use of embodiment 449 wherein the medicament is configured to be administered in combination with a second therapeutic agent.
Embodiment 451. Use of a pharmaceutically acceptable composition of embodiment 429 in the manufacturing of a medicament for treating an MTAP-deficient and/or an MTA-accumulating disease in a subject in need thereof.
Embodiment 452. The use of any one of embodiments 449 to 451 wherein the disease is a proliferating disease.
Embodiment 453. The use of embodiment 452 wherein the disease is an MTAP-deficient and/or MTA-accumulating cancer.
Embodiment 454. The use of embodiment 453 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one membered of the group is present in, employed in, or otherwise relevant to a given product or process. In some embodiments, more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or embodiments of the invention, is/are referred to as comprising particular elements and/or features, certain embodiments of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment that falls within the prior art may be explicitly excluded from any one or more of the claims. Because such embodiments are deemed to be known to one of ordinary skill in the art, they may be excluded even if the exclusion is not set forth explicitly herein. Any particular embodiment be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (A) or Formula (I) or a pharmaceutically acceptable salt thereof, wherein: 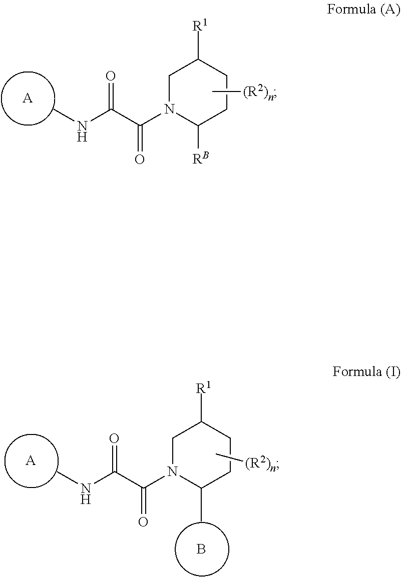
Ring A is an optionally substituted fused bicyclic 8-10 membered heteroaryl ring system containing at least one nitrogen atom, wherein the 8-10 membered refers to the total number of atoms in the fused system;
R^{B} is an optionally substituted C₁-C₆ alkyl;
Ring B is selected from the group consisting of —C₃-C₁₀ carbocyclyl, 3-10 membered heterocyclyl, C₆-C₁₀ aryl, 5-10 membered heteroaryl, wherein each carbocyclyl, heterocyclyl, aryl and heteroaryl is optionally substituted at any available position;
each R¹ is independently absent or selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, 5-6-membered monocyclic heteroaryl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(R^{a1})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —CH₂C(═O)N(R^{a2})₂—C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ or two instances of R² together with the atom or atoms to which they are attached can be taken together to form a 3-10 membered cycloalkyl or heterocyclyl ring (e.g., a ring that together with the piperidine ring of Formula (I) or Formula (A) can form a bridged, fused or spiro bicyclic heterocyclic ring);
each R^{a1} and R^{a2} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3;
provided that:
(i) when the compound is of Formula (A), Ring A is a 3-pyridine fused with a 5-membered heteroaryl via 2 carbon atoms; and
(ii) the compound is not one of compounds a. to d. or a pharmaceutically acceptable salt thereof:
a. N-(8-fluoroquinolin-3-yl)-2-oxo-2-(2-phenylpiperidin-1-yl)acetamide 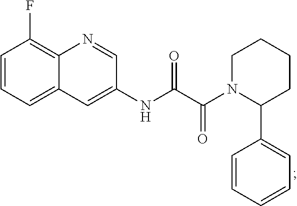 [SMILES: O=C(Nc1cnc2c(F)cccc2c1)C(=O)N1CCCCC1c1ccccc1]
b. 2-(2-(1H-pyrazol-4-yl)piperidin-1-yl)-N-(1H-indol-4-yl)-2-oxoacetamide 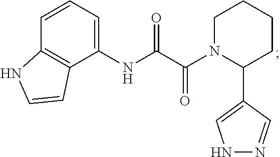 [SMILES: O=C(Nc1cccc2[nH]ccc12)C(=O)N1CCCCC1c1cn[nH]c1]
c. 2-(2-(4-bromophenyl)piperidin-1-yl)-2-oxo-N-(pyrazolo[1,5-a]pyrimidin-3-yl)acetamide 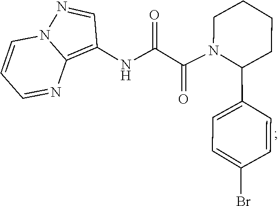 [SMILES: O=C(Nc1cnn2cccnc12)C(=O)N1CCCCC1c1ccc(Br)cc1]
d. 2-(2-(1H-pyrazol-4-yl)piperidin-1-yl)-N-(1H-indazol-5-yl)-2-oxoacetamide

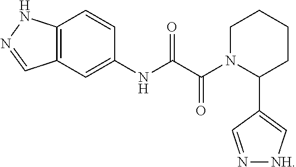 [SMILES: O=C(Nc1ccc2[nH]ncc2c1)C(=O)N1CCCCC1c1cn[nH]c1]

2. The compound of claim 1, or a pharmaceutically acceptable salt thereof, wherein R^{B} is selected from -Me, -Et, -ⁱPr and -^{t}Bu.

3. The compound of claim 1, or a pharmaceutically acceptable salt thereof, wherein the compound is of Formula (I):

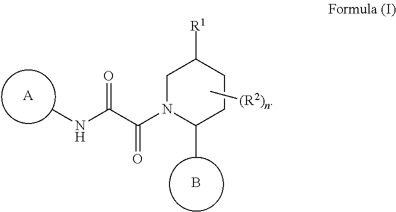

4. The compound of claim 1 or 3, or a pharmaceutically acceptable salt thereof, wherein R¹ is not H and Ring B and R¹ are in a trans relative configuration.

5. The compound of any one of claims 1, 3 and 4, or a pharmaceutically acceptable salt thereof, wherein the moiety represented as 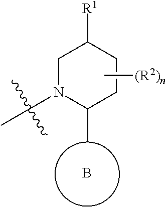
is selected from the group consisting of:

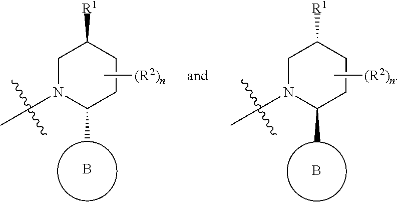

6. The compound of claim 5, or a pharmaceutically acceptable salt thereof, wherein the compound is of Formula (Ia):

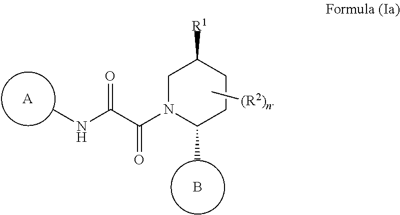

7. The compound of any one of claims 1 to 6, or a pharmaceutically acceptable salt thereof, wherein Ring A is a fused bicyclic 8-10 membered heteroaryl ring containing at least one nitrogen atom, substituted at any available positions with 0, 1, 2 or 3 instances of R⁴, wherein the 8-10 membered refers to the total number of atoms in the fused system;
each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂; and
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl).

8. The compound of any one of claims 1 to 6, or a pharmaceutically acceptable salt thereof, wherein Ring A is selected from the group consisting of: 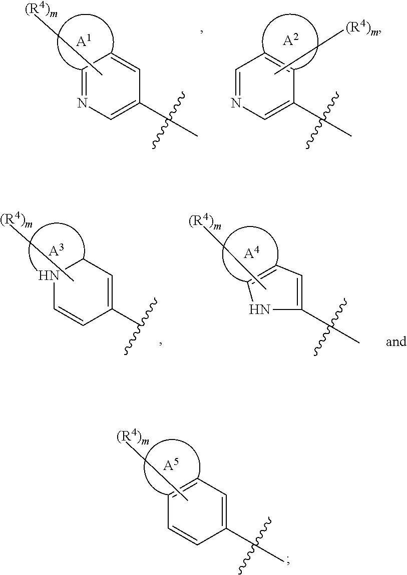
wherein
each of rings A¹, A² and A⁴ is independently 4-6 membered carbocyclyl, 4-6 membered heterocyclyl, 5-6 membered heteroaryl or phenyl;
each ring A³ is independently a 4-6 membered heterocyclyl or 5-6 membered heteroaryl, wherein the heterocyclyl and heteroaryl contain at least one nitrogen atom;
each ring A⁵ is independently a 5-6 membered heteroaryl, wherein the heteroaryl contains at least one nitrogen atom;
each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —ORD, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl); and
m is 0, 1, 2 or 3.

9. The compound of claim 7, or a pharmaceutically acceptable salt thereof, wherein Ring A is selected from the group consisting of:

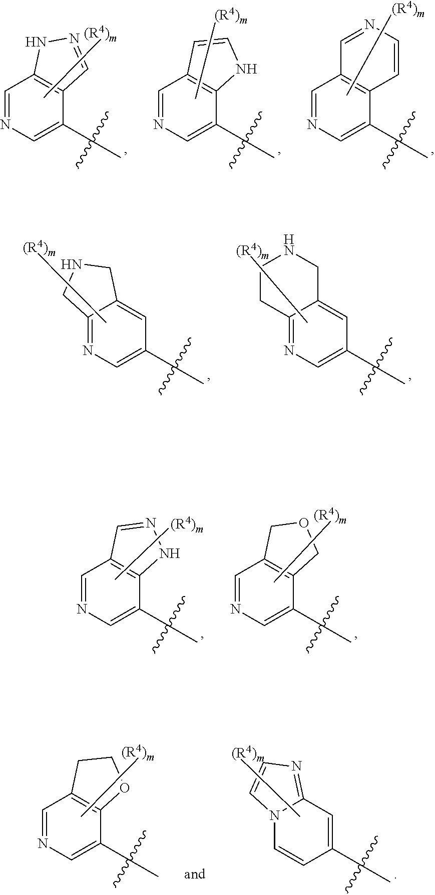

10. The compound of claim 7, or a pharmaceutically acceptable salt thereof, wherein Ring A is

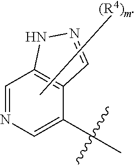

11. The compound of claim 7, or a pharmaceutically acceptable salt thereof, wherein Ring A is

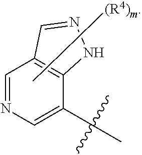

12. The compound of claim 7, or a pharmaceutically acceptable salt thereof, wherein Ring A is

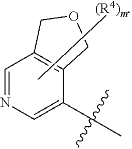

13. The compound of claim 7, or a pharmaceutically acceptable salt thereof, wherein Ring A is selected from the group consisting of:

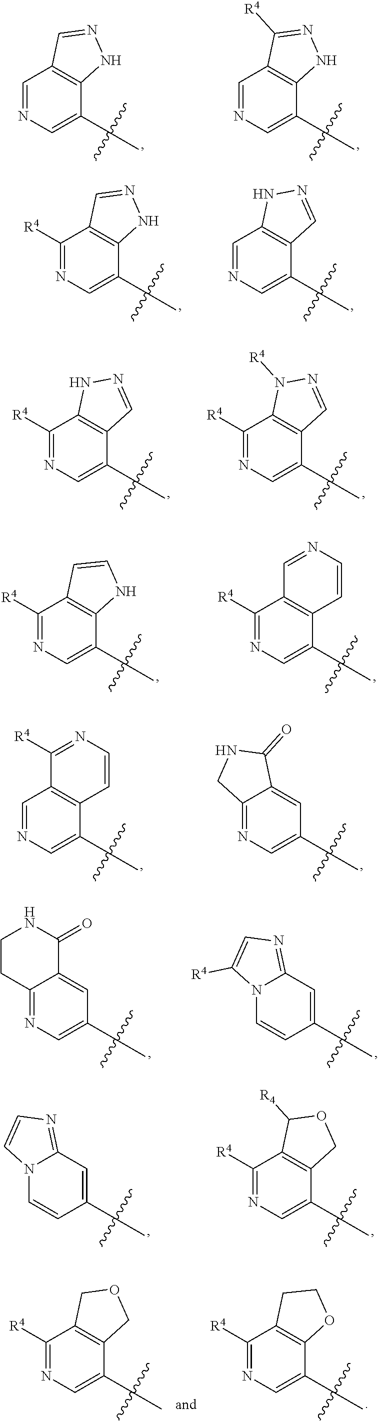

14. The compound of any one of claims 7 to 13, or a pharmaceutically acceptable salt thereof, wherein each R⁴ is independently selected from the group consisting of ═O, —C₁-C₆ alkyl and —N(R^{a4})₂.

15. The compound of any one of claims 7 to 14, or a pharmaceutically acceptable salt thereof, wherein each R^{a4} is independently selected from the group consisting of H and C₁-C₆ alkyl.

16. The compound of any one of claims 7 to 14, or a pharmaceutically acceptable salt thereof, wherein each R^{a4} is H.

17. The compound of any one of claims 7 to 13, or a pharmaceutically acceptable salt thereof, wherein each R⁴ is independently —NHCH₃, —NH₂ or -Me.

18. The compound of any one of claims 7 to 13, or a pharmaceutically acceptable salt thereof, wherein each R⁴ is independently —NH₂ or -Me.

19. The compound of any claim 7, or a pharmaceutically acceptable salt thereof, wherein Ring A is selected from the group consisting of:

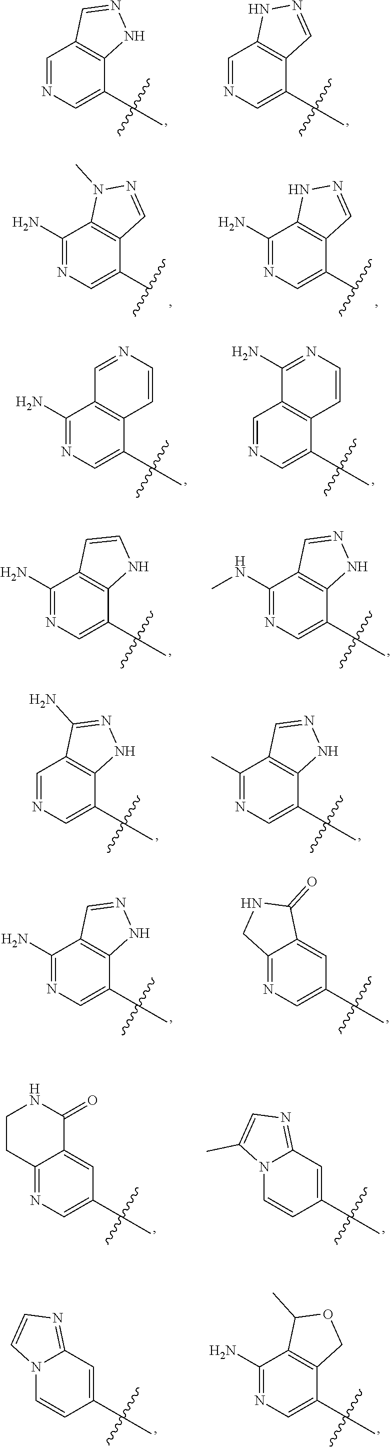

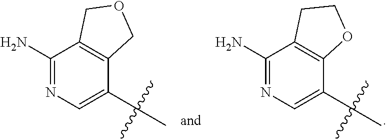 [SMILES: *c1cnc(N)c2c1COC2 ; *c1cnc(N)c2c1OCC2]

20. The compound of claim 7, or a pharmaceutically acceptable salt thereof, wherein Ring A is

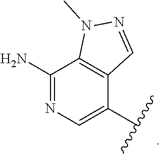 [SMILES: *c1cnc(N)c2c1cnn2C]

21. The compound of claim 7, or a pharmaceutically acceptable salt thereof, wherein Ring A is

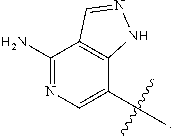 [SMILES: *c1cnc(N)c2cn[nH]c12]

22. The compound of any one of claims 1 to 21, or a pharmaceutically acceptable salt thereof, wherein each R¹ is independently selected from the group consisting of H, -Me and -Et.

23. The compound of any one of claims 1 to 21, or a pharmaceutically acceptable salt thereof, wherein R¹ is -Me.

24. The compound of any one of claims 1 to 23, or a pharmaceutically acceptable salt thereof, wherein each R² is independently selected from the group consisting of halo (e.g., —Cl), —C₁-C₆ alkyl (e.g., -Me, -Et, -Pr, -ⁱPr, -ⁿBu, -^{t}Bu, -sec-Bu, -iso-Bu), —C₁-C₆ haloalkyl (e.g., —CF₃, —CHF₂), —C₃-C₉ cycloalkyl (e.g., cyclopropyl, cyclobutyl), and —OCH₃.

25. The compound of any one of claims 1 to 23, or a pharmaceutically acceptable salt thereof, wherein each R² is independently selected from the group consisting of —F, -Me, —CF₃ and —OCH₃.

26. The compound of any one of claims 1 to 25, or a pharmaceutically acceptable salt thereof, wherein Ring B is selected from the group consisting of C₆-C₁₀ aryl and 5-10 membered heteroaryl, wherein the aryl and heteroaryl are optionally substituted at any available position.

27. The compound of any one of claims 1 to 25, or a pharmaceutically acceptable salt thereof, wherein Ring B is selected from the group consisting of phenyl, naphthalenyl, C₃-C₇ monocyclic cycloalkyl, 3-7 membered monocyclic heterocyclyl, 5-6 membered monocyclic heteroaryl, and 8-10 membered bicyclic heteroaryl, each optionally substituted at any available position.

28. The compound of any one of claims 1 to 25, or a pharmaceutically acceptable salt thereof, wherein Ring B is selected from the group consisting of cyclopropyl, cyclobutyl, cyclopentyl, cyclohexyl, tetrahydrofuranyl, tetrahydropyranyl, thiophenyl, oxazolyl, pyrazolyl, phenyl, naphthalenyl, pyridinyl, 1H-indazolyl, 2H-indazolyl, quinolinyl, isoquinolinyl, 1,2,3,4-tetrahydroquinolinyl, 1,2,3,4-tetrahydroisoquinolinyl, 2,3-dihydrobenzofuranyl and benzo[d]thiazolyl, each optionally substituted at any available position.

29. The compound of any one of claims 1 to 25, or a pharmaceutically acceptable salt thereof, wherein Ring B is selected from the group consisting of cyclopropyl, cyclobutyl, cyclopentyl, cyclohexyl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H indazol-5-yl, 1H indazol-4-yl, 2H-indazol-6-yl, 2H-indazol-5-yl, quinolin-6-yl, quinolin-7-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl and benzo[d]thiazol-5-yl, each optionally substituted at any available position.

30. The compound of any one of claims 1 to 25, or a pharmaceutically acceptable salt thereof, wherein Ring B is C₃-C₇ monocyclic cycloalkyl, optionally substituted at any available position.

31. The compound of any one of claims 1 to 25, or a pharmaceutically acceptable salt thereof, wherein Ring B is selected from the group consisting of cyclopropyl, cyclobutyl, cyclopentyl and cyclohexyl, each optionally substituted at any available position.

32. The compound of any one of claims 1 to 25, or a pharmaceutically acceptable salt thereof, wherein Ring B is 3-7 membered monocyclic heterocyclyl containing 1 or 2 heteroatoms independently selected from N, O and S or oxidized forms thereof, wherein the heterocyclyl is optionally substituted at any available position.

33. The compound of any one of claims 1 to 25, or a pharmaceutically acceptable salt thereof, wherein Ring B is selected from the group consisting of tetrahydrofuranyl and tetrahydropyranyl, each optionally substituted at any available position.

34. The compound of any one of claims 1 to 25, or a pharmaceutically acceptable salt thereof, wherein Ring B is a 5-6 membered monocyclic heteroaryl containing 1, 2 or 3 heteroatoms independently selected from N, O and S, optionally substituted at any available position.

35. The compound of any one of claims 1 to 25, or a pharmaceutically acceptable salt thereof, wherein Ring B is selected from the group consisting of pyrazolyl, thiophenyl, oxazolyl and pyridinyl, each optionally substituted at any available position.

36. The compound any one of claims 1 to 25, or a pharmaceutically acceptable salt thereof, wherein Ring B is selected from the group consisting of cyclopentyl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H-indazol-5-yl, 2H-indazol-6-yl, 2H-indazol-5-yl, quinolin-6-yl, quinolin-7-yl, isoquinolin-6-yl, benzo[d]thiazol-5-yl, each optionally substituted.

37. The compound of any one of claims 1 to 25, or a pharmaceutically acceptable salt thereof, wherein Ring B is selected from the group consisting of naphthalenyl, phenyl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each optionally substituted at any available position.

38. The compound of any one of claims 1 to 25, or a pharmaceutically acceptable salt thereof, wherein Ring B is naphthalenyl or phenyl, each optionally substituted at any available position.

39. The compound of any one of claims 1 to 25, or a pharmaceutically acceptable salt thereof, wherein Ring B is phenyl, optionally substituted at any available position.

40. The compound of any one of claims 1 to 25, or a pharmaceutically acceptable salt thereof, wherein Ring B is an 8-10 membered bicyclic heteroaryl, wherein the bicyclic heteroaryl is optionally substituted at any available position.

41. The compound of any one of claims 1 to 25, or a pharmaceutically acceptable salt thereof, wherein Ring B is selected from the group consisting of 1H-indazolyl, 2H-indazolyl, quinolinyl, isoquinolinyl and benzo[d]thiazolyl, each optionally substituted at any available position.

42. The compound of any one of claims 1 to 25, or a pharmaceutically acceptable salt thereof, wherein Ring B is independently selected from the group consisting of 1H-indazol-5-yl, 1H indazol-4-yl, 2H-indazol-6-yl, 2H-indazol-5-yl, quinolin-6-yl, quinolin-7-yl, isoquinolin-6-yl and benzo[d]thiazol-5-yl, each optionally substituted at any available position.

43. The compound of any one of claims 1 to 25, or a pharmaceutically acceptable salt thereof, wherein Ring B is substituted at available positions with 0, 1, 2 or 3 instances of R³;
each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3}) R^{a3}, —NR^{a3}S(═O)₂R^{a3} and —S(═O)₂N(R^{a3})₂, wherein each alkyl, cycloalkyl, heteroalkyl, heterocyclyl, aryl, heteroaryl, cycloalkylalkyl, heterocyclylalkyl, arylalkyl and heteroarylalkyl of R³ is optionally substituted at any available position (e.g., substituted with 0, 1, 2, 3, 4, or 5 instances of R⁶, wherein each R⁶ is independently selected from the group consisting of —C₁-C₆ alkyl, —C₃-C₉ cycloalkyl, 3-10 membered heterocyclyl, —OH, ═O, halo, —OC₁-C₆ alkyl, —C₁-C₆ haloalkyl, —C(═O)C₁-C₆ alkyl, —N(C₁-C₆ alkyl)₂, —C₁-C₆ heteroalkyl, and —NHC(═O)C₁-C₆ alkyl, wherein one or more hydrogens of the —C₁-C₆ alkyl can be replaced with deuterium); and
each R^{a3} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of —O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR^{b}, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R^{b}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, -Pr, -ⁱPr, -ⁿBu, -^{t}Bu, -sec-Bu, -iso-Bu)), and C₃-C₉ cycloalkyl (e.g., cyclopropyl, cyclobutyl, cyclopentyl, cyclohexyl).

44. The compound of claim 43, or a pharmaceutically acceptable salt thereof, wherein Ring B is selected from the group consisting of phenyl, naphthalenyl, C₃-C₇ monocyclic cycloalkyl, 3-7 membered monocyclic heterocyclyl, 5-6 membered monocyclic heteroaryl, and 8-10 membered bicyclic heteroaryl, each substituted at available positions with 0, 1, 2 or 3 instances of R³.

45. The compound of claim 43, or a pharmaceutically acceptable salt thereof, wherein Ring B is selected from the group consisting of C₆-C₁₀ aryl and 8-10 membered bicyclic heteroaryl wherein the aryl and heteroaryl are substituted at available positions with 0, 1, 2 or 3 instances of R³.

46. The compound of claim 43, or a pharmaceutically acceptable salt thereof, wherein Ring B is selected from the group consisting of cyclopropyl, cyclobutyl, cyclopentyl, cyclohexyl, tetrahydrofuranyl, tetrahydropyranyl, thiophenyl, oxazolyl, pyrazolyl, phenyl, naphthalenyl, pyridinyl, 1H-indazolyl, 2H-indazolyl, quinolinyl, isoquinolinyl, 1,2,3,4-tetrahydroquinolinyl, 1,2,3,4-tetrahydroisoquinolinyl, 2,3-dihydrobenzofuranyl and benzo[d]thiazolyl, each substituted at available positions with 0, 1, 2 or 3 instances of R³.

47. The compound of claim 43, or a pharmaceutically acceptable salt thereof, wherein Ring B is selected from the group consisting of cyclopropyl, cyclobutyl, cyclopentyl, cyclohexyl, tetrahydrofuran-3-yl, tetrahydropyran-2-yl, tetrahydropyran-3-yl, tetrahydropyran-4-yl, pyrazol-5-yl, pyrazol-3-yl, thiophen-3-yl, oxazol-5-yl, pyridin-3-yl, pyridin-4-yl, phenyl, naphthalen-2-yl, 1H indazol-5-yl, 1H indazol-4-yl, 2H-indazol-6-yl, 2H-indazol-5-yl, quinolin-6-yl, quinolin-7-yl, isoquinolin-6-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl, 2,3-dihydrobenzofuran-7-yl and benzo[d]thiazol-5-yl, each substituted at available positions with 0, 1, 2 or 3 instances of R³.

48. The compound of claim 43, or a pharmaceutically acceptable salt thereof, wherein Ring B is C₃-C₇ monocyclic cycloalkyl, substituted at available positions with 0, 1, 2 or 3 instances of R³.

49. The compound of claim 43, or a pharmaceutically acceptable salt thereof, wherein Ring B is selected from the group consisting of cyclopropyl, cyclobutyl, cyclopentyl and cyclohexyl, each substituted at available positions with 0, 1, 2 or 3 instances of R³.

50. The compound of claim 43, or a pharmaceutically acceptable salt thereof, wherein Ring B is 3-7 membered monocyclic heterocyclyl containing 1 or 2 heteroatoms independently selected from N, O and S or oxidized forms thereof, wherein the heterocyclyl is substituted at available positions with 0, 1, 2 or 3 instances of R³.

51. The compound of claim 43, or a pharmaceutically acceptable salt thereof, wherein Ring B is selected from the group consisting of tetrahydrofuranyl and tetrahydropyranyl, each substituted at available positions with 0, 1, 2 or 3 instances of R³.

52. The compound of claim 43, or a pharmaceutically acceptable salt thereof, wherein Ring B is a 5-6 membered monocyclic heteroaryl containing 1, 2 or 3 heteroatoms independently selected from N, O and S, substituted at available positions with 0, 1, 2 or 3 instances of R³.

53. The compound of claim 43, or a pharmaceutically acceptable salt thereof, wherein Ring B is selected from the group consisting of pyrazolyl, thiophenyl, oxazolyl and pyridinyl, each substituted at available positions with 0, 1, 2 or 3 instances of R³.

54. The compound of claim 43, or a pharmaceutically acceptable salt thereof, wherein Ring B is selected from the group consisting of naphthalenyl, phenyl, 1,2,3,4-tetrahydroquinolinyl, 1,2,3,4-tetrahydroisoquinolinyl, 2,3-dihydrobenzofuranyl), wherein the aryl is substituted at available positions with 0, 1, 2 or 3 instances of R³.

55. The compound of claim 43, or a pharmaceutically acceptable salt thereof, wherein Ring B is selected from the group consisting of phenyl, naphthalen-2-yl, 1,2,3,4-tetrahydroquinolin-6-yl, 1,2,3,4-tetrahydroisoquinolin-6-yl, 1,2,3,4-tetrahydroisoquinolin-7-yl, 2,3-dihydrobenzofuran-4-yl, 2,3-dihydrobenzofuran-5-yl and 2,3-dihydrobenzofuran-7-yl, each substituted at available positions with 0, 1, 2 or 3 instances of R³.

56. The compound of claim 43, or a pharmaceutically acceptable salt thereof, wherein Ring B is phenyl substituted at available positions with 0, 1, 2 or 3 instances of R³.

57. The compound of claim 43, or a pharmaceutically acceptable salt thereof, wherein Ring B is an 8-10 membered bicyclic heteroaryl, wherein the bicyclic heteroaryl is substituted with 0, 1, 2 or 3 instances of R³.

58. The compound of claim 43, or a pharmaceutically acceptable salt thereof, wherein Ring B is selected from the group consisting of 1H-indazolyl, 2H-indazolyl, quinolinyl, isoquinolinyl and benzo[d]thiazolyl, each substituted with 0, 1, 2 or 3 instances of R³.

59. The compound of claim 43, or a pharmaceutically acceptable salt thereof, wherein Ring B is independently selected from the group consisting of 1H-indazol-5-yl, 1H indazol-4-yl, 2H-indazol-6-yl, 2H-indazol-5-yl, quinolin-6-yl, quinolin-7-yl, isoquinolin-6-yl and benzo[d]thiazol-5-yl, each substituted at available positions with 0, 1, 2 or 3 instances of R³.

60. The compound of claim 43, or a pharmaceutically acceptable salt thereof, wherein Ring B is selected from the group consisting of:

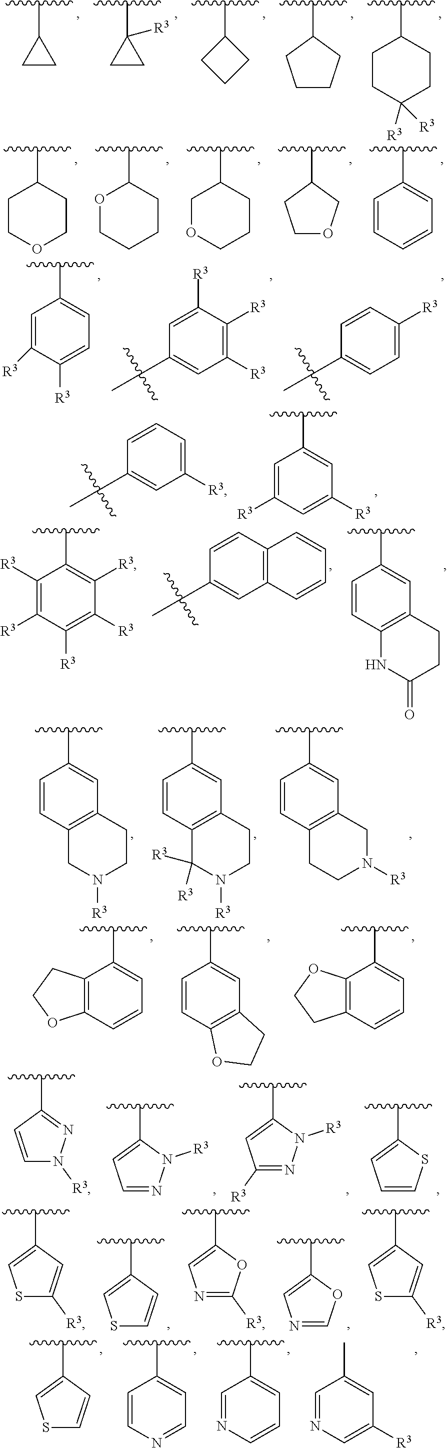

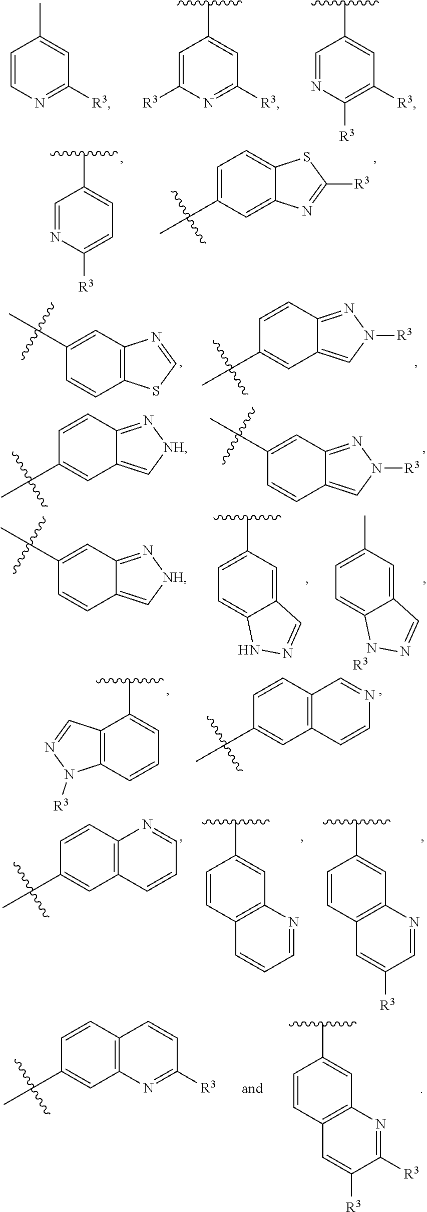

61. The compound of claim 43, or a pharmaceutically acceptable salt thereof, wherein Ring B is selected from the group consisting of:

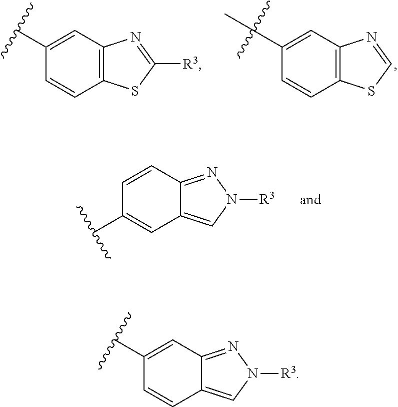

62. The compound of any one of claims 43 to 61, or a pharmaceutically acceptable salt thereof, wherein each R³ is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, cycloalkylalkyl, heterocyclylalkyl, —OR^{a3}, —N(R^{a3})₂, —NR^{a3}C(═O)R^{a3}, wherein each alkyl, cycloalkyl, heterocyclyl, cycloalkylalkyl and heterocyclylalkyl is optionally substituted (e.g., with 0, 1, 2, 3, 4, or 5 instances of R⁶; and
each R^{a3} is independently selected from the group consisting of H, —C₁-C₆ alkyl, —C₁-C₆ haloalkyl, heterocyclylalkyl substituted with 0 or 1 instances of -Me, —C₁-C₆ heteroalkyl substituted with 0 or 1 instances of ═O, C₃-C₉ cycloalkyl, and 3-10 membered heterocyclyl substituted with 0 or 1 instances of ═O, -Me or a combination thereof.

63. The compound of any one of claims 43 to 61, or a pharmaceutically acceptable salt thereof, wherein each R³ is independently selected from the group consisting of D, —CN, halo (e.g., —F, —Cl, Br), —C₁-C₆ alkyl (e.g., -Me, -Et, -Pr, -ⁱPr, -sec-Bu, -^{t}Bu, —CH₂(CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂)—C₁-C₆ haloalkyl, (e.g., —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrolidinyl, piperidinyl, tetrahydropyridinyl (e.g., 1,2,3,6 tetrahydropyridinyl), piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-piperidinyl, —CH₂-pyrolidinyl, —CH(CH₃)CH₂-pyrolidinyl, —(CH₂)₂-pyrolidinyl, —(CH₂)₃-pyrolidinyl), —OR^{a3} (e.g., —OH, —OCH₃, —OCH₂CH₃, —OCH₂CH₂N(CH₃)₂, —OCH₂CH(CH₃)N(CH₃)₂, —OCF₃, —OCHF₂, —O-piperidinyl, —OCH₂-pyrrolidinyl), —NR^{a3})₂, —NR^{a3}C(═O)R^{a3} (e.g., —NHC(═O)CH₃), —NHC(═O)CH₂CH₂N(CH₃)₂), wherein each alkyl, cycloalkyl, heterocyclyl, cycloalkylalkyl, and heterocyclylalkyl is optionally substituted (e.g., with 0, 1, 2, 3, 4, or 5 instances of R⁶; and
each R^{a3} is independently selected from the group consisting of H, —C₁-C₆ alkyl, (e.g., -Me, -Et, -Pr, -ⁱPr, -sec-Bu, -^{t}Bu), —C₁-C₆ haloalkyl (e.g., —CF₃, —CHF₂, —CH₂CF₃), —C₁-C₆ heteroalkyl substituted with 0 or 1 instances of ═O (e.g., —CH₂CH₂N(CH₃)₂, —CH₂CH(CH₃)N(CH₃)₂, —CH₂C(═O)N(CH₃)₂, —CH(CH₃)CH₂N(CH₃)₂, —CH(CH₃)C(═O)N(CH₃)₂)), C₃-C₉ cycloalkyl (e.g., cyclopropyl, cyclobutyl, cyclopentyl, cyclohexyl), heterocyclylalkyl (e.g., —CH₂(N-methyl pyrrolidin-2-yl)) and 3-10 membered heterocyclyl (e.g., piperidinyl, e.g., N-methyl piperidin-4-yl).

64. The compound of any one of claims 43 to 63, or a pharmaceutically acceptable salt thereof, wherein each R^{a3} is independently selected from the group consisting of H, -Me, -Et, -Pr, -ⁱPr, -sec-Bu, -^{t}Bu, —CF₃, —CHF₂, —CH₂CF₃, —CH₂(N-methyl pyrrolidin-2-yl), N-methyl piperidin-4-yl, —CH₂CH(CH₃)N(CH₃)₂, cyclopropyl and —CH₂CH₂N(CH₃)₂.

65. The compound of any one of claims 43 to 61, or a pharmaceutically acceptable salt thereof, wherein each R³ is independently selected from the group consisting of D, —CN, -Me, -Et, -Pr, —F, —Cl, —CF₃, —N(CH₃)₂, —N(CH₂CH₃)₂, —N(CH₃)CH₂CH₃, —OMe, -OEt, —O-cyclopropyl, —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂C(CH₃)₂N(CH₃)₂, cyclopropyl, cyclobutyl, cyclohexyl, tetrahydrofuranyl, tetrahydropyranyl, oxetanyl, morpholinyl, pyrolidinyl, piperidinyl, tetrahydropyridinyl (e.g., 1,2,3,6 tetrahydropyridinyl), piperidin-2-onyl, piperazinyl, azetidinyl, 2-azaspiro[3.3]heptanyl, 2-azabicyclo[2.2.2]octanyl, 2-azabicyclo[2.2.1]heptanyl, 1-azabicyclo[2.2.1]heptanyl, —CH₂-piperidinyl, —CH₂-pyrolidinyl, —CH(CH₃)CH₂-pyrolidinyl, —(CH₂)₂-pyrolidinyl, —(CH₂)₃-pyrolidinyl, —OCH₂CH₂N(CH₃)₂, —OCH₂CH(CH₃)N(CH₃)₂, —OCH₂(pyrrolidinyl), —Opiperidinyl, and —NHC(═O)CH₂CH₂N(CH₃)₂), wherein each cyclopropyl, cyclobutyl, cyclohexyl, tetrahydrofuranyl, tetrahydropyranyl, oxetanyl, morpholinyl, pyrolidinyl, piperidinyl, tetrahydropyridinyl, piperidin-2-onyl, piperazinyl, azetidinyl, 2-azaspiro[3.3]heptanyl, 2-azabicyclo[2.2.2]octanyl, 1-azabicyclo[2.2.1]heptanyl, 2-azabicyclo[2.2.1]heptanyl, —CH₂-piperidinyl, —CH₂-pyrolidinyl, —CH(CH₃)CH₂-pyrolidinyl, —(CH₂)₂-pyrolidinyl, —(CH₂)₃-pyrolidinyl, —CH(CH₃)CH₂-pyrolidinyl and —(CH₂)₃-pyrolidinyl is optionally substituted (e.g., with 0, 1, 2, 3, 4, or 5 instances of R⁶).

66. The compound of any one of claims 43 to 61, or a pharmaceutically acceptable salt thereof, wherein R³ is selected from the group consisting of 3-8 membered monocyclic heterocyclyl, and 5-10 membered bicyclic heterocyclyl, wherein each heterocyclyl is optionally substituted ((e.g., substituted with 0, 1, 2, 3, 4, or 5 instances of R⁶).

67. The compound of any one of claims 43 to 61, or a pharmaceutically acceptable salt thereof, wherein each R⁶ is independently selected from the group consisting of -Me, -Et, -CD₃, cyclopropyl, oxetan-3-yl, —OH, ═O, —N(CH₃)₂ and —CH₂N(CH₃)₂.

68. The compound of any one of claims 43 to 61, or a pharmaceutically acceptable salt thereof, wherein R³ is selected from the group consisting of -D, —CN, -Me, -Et, -Pr, —OMe, -OEt, —F, —Cl, —CF₃,

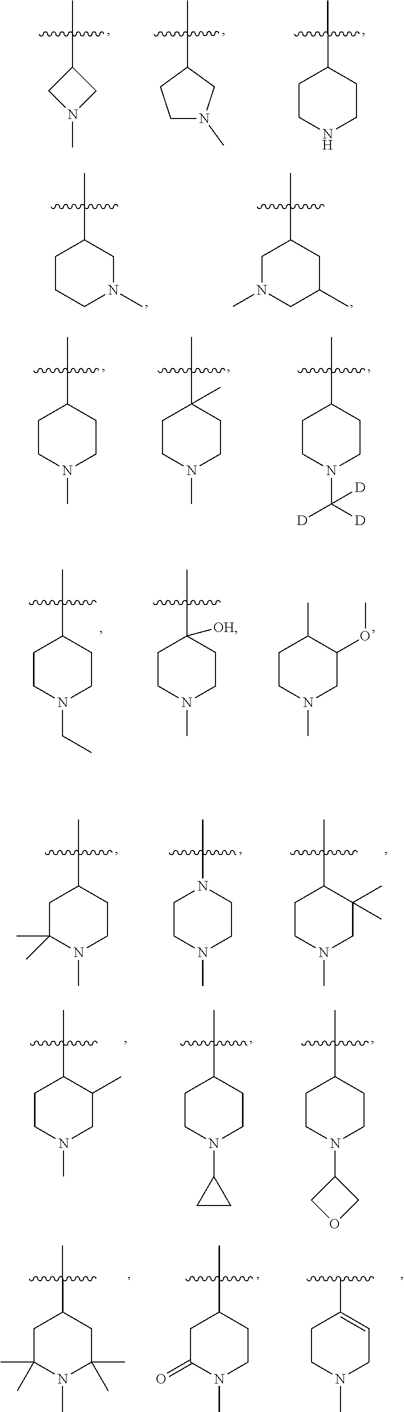

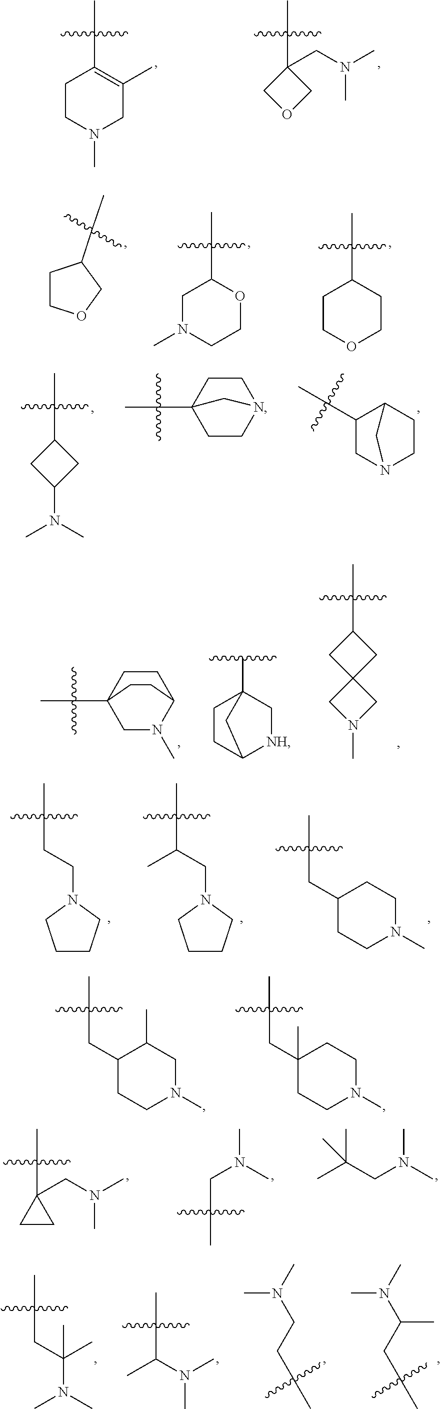

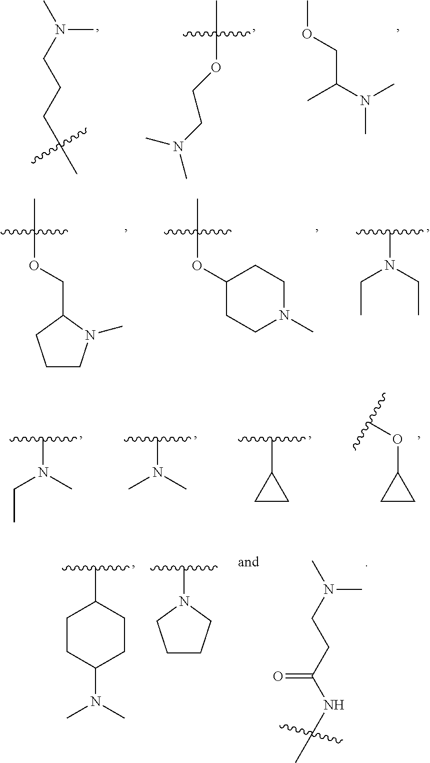

69. The compound of any one of claims 1 to 68, or a pharmaceutically acceptable salt thereof, wherein the compound is selected from the group consisting of: 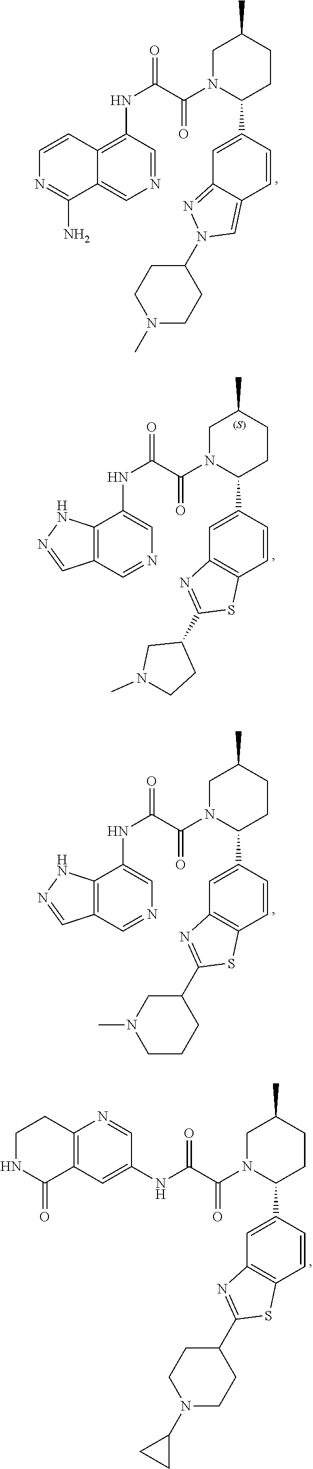 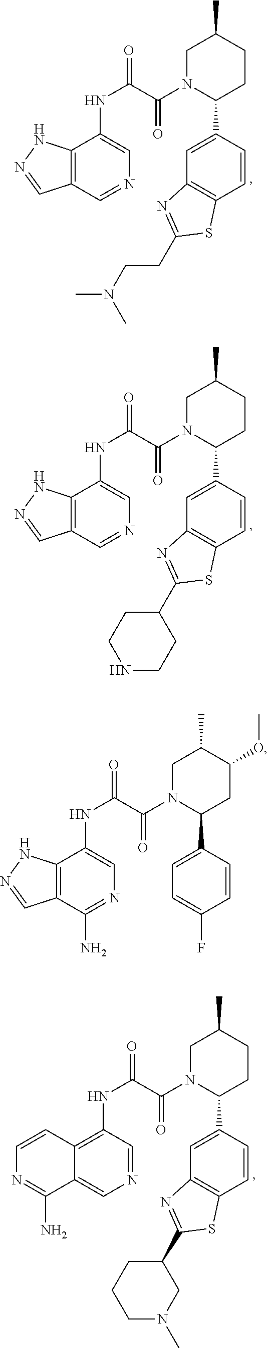 [SMILES: C[C@H]1CC[C@H](c2ccc3sc(CCN(C)C)nc3c2)N(C(=O)C(=O)Nc2cncc3cn[nH]c23)C1 ; C[C@H]1CC[C@H](c2ccc3sc(C4CCNCC4)nc3c2)N(C(=O)C(=O)Nc2cncc3cn[nH]c23)C1 ; CO[C@@H]1C[C@@H](c2ccc(F)cc2)N(C(=O)C(=O)Nc2cnc(N)c3cn[nH]c23)C[C@@H]1C ; C[C@H]1CC[C@H](c2ccc3sc([C@@H]4CCCN(C)C4)nc3c2)N(C(=O)C(=O)Nc2cncc3c(N)nccc23)C1] 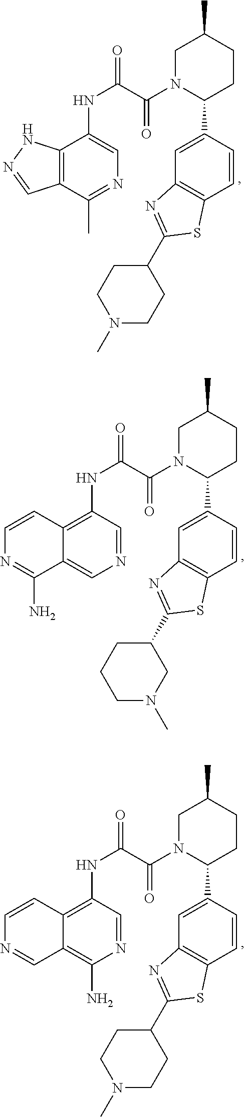 [SMILES: Cc1ncc(NC(=O)C(=O)N2C[C@@H](C)CC[C@@H]2c2ccc3sc(C4CCN(C)CC4)nc3c2)c2[nH]ncc12 ; C[C@H]1CC[C@H](c2ccc3sc([C@H]4CCCN(C)C4)nc3c2)N(C(=O)C(=O)Nc2cncc3c(N)nccc23)C1 ; C[C@H]1CC[C@H](c2ccc3sc(C4CCN(C)CC4)nc3c2)N(C(=O)C(=O)Nc2cnc(N)c3cnccc23)C1] 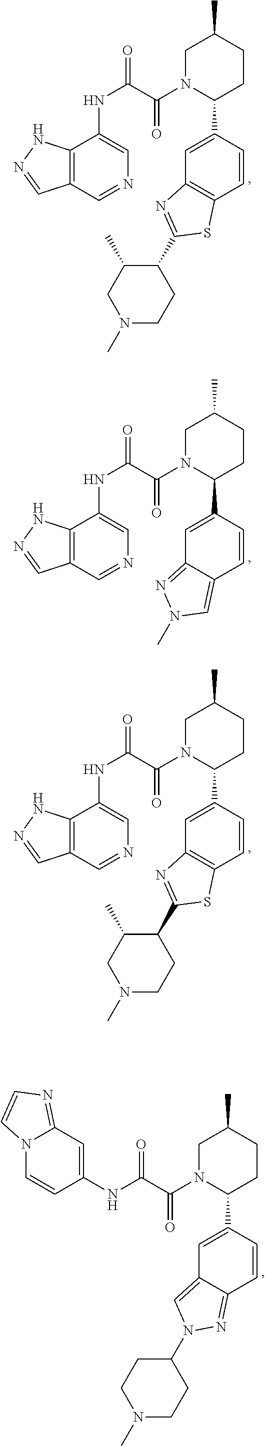 [SMILES: C[C@H]1CC[C@H](c2ccc3sc([C@@H]4CCN(C)C[C@@H]4C)nc3c2)N(C(=O)C(=O)Nc2cncc3cn[nH]c23)C1 ; C[C@@H]1CC[C@@H](c2ccc3cn(C)nc3c2)N(C(=O)C(=O)Nc2cncc3cn[nH]c23)C1 ; C[C@H]1CC[C@H](c2ccc3sc([C@H]4CCN(C)C[C@@H]4C)nc3c2)N(C(=O)C(=O)Nc2cncc3cn[nH]c23)C1 ; C[C@H]1CC[C@H](c2ccc3nn(C4CCN(C)CC4)cc3c2)N(C(=O)C(=O)Nc2ccn3ccnc3c2)C1] 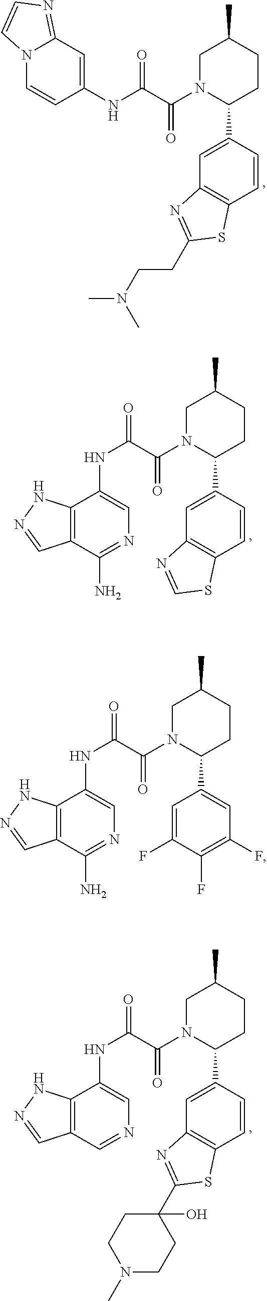 [SMILES: C[C@H]1CC[C@H](c2ccc3sc(CCN(C)C)nc3c2)N(C(=O)C(=O)Nc2ccn3ccnc3c2)C1 ; C[C@H]1CC[C@H](c2ccc3scnc3c2)N(C(=O)C(=O)Nc2cnc(N)c3cn[nH]c23)C1 ; C[C@H]1CC[C@H](c2cc(F)c(F)c(F)c2)N(C(=O)C(=O)Nc2cnc(N)c3cn[nH]c23)C1 ; C[C@H]1CC[C@H](c2ccc3sc(C4(O)CCN(C)CC4)nc3c2)N(C(=O)C(=O)Nc2cncc3cn[nH]c23)C1] 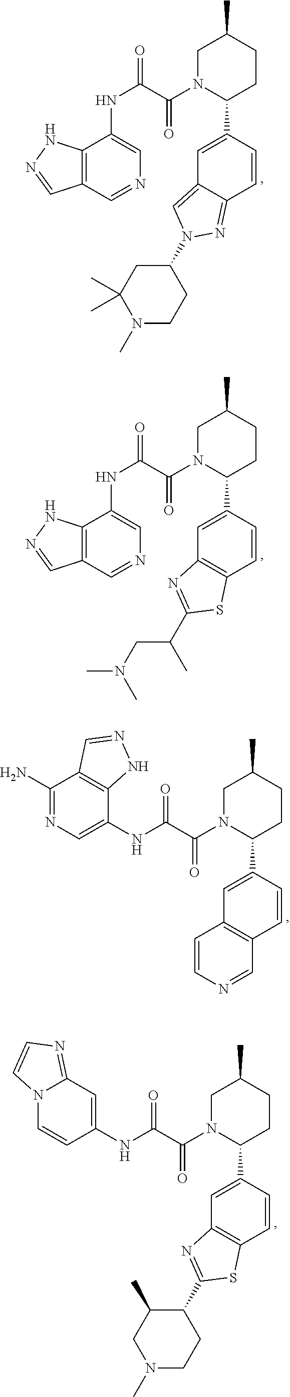 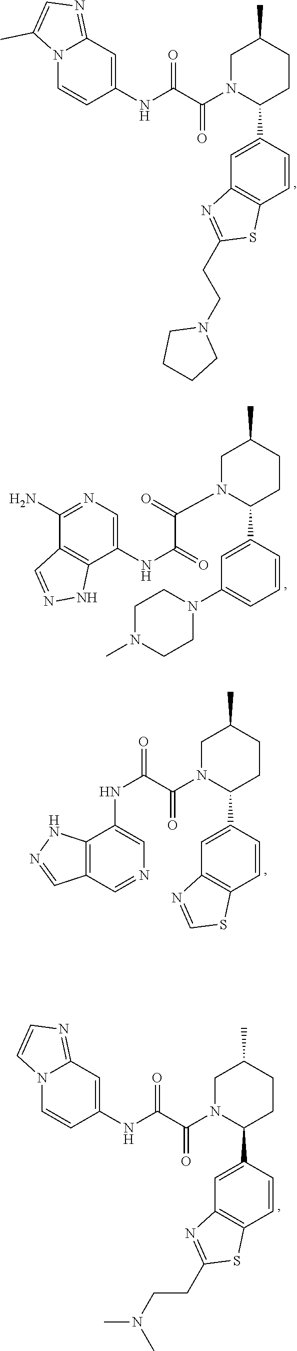 [SMILES: Cc1cnc2cc(NC(=O)C(=O)N3C[C@@H](C)CC[C@@H]3c3ccc4sc(CCN5CCCC5)nc4c3)ccn12 ; C[C@H]1CC[C@H](c2cccc(N3CCN(C)CC3)c2)N(C(=O)C(=O)Nc2cnc(N)c3cn[nH]c23)C1 ; C[C@H]1CC[C@H](c2ccc3scnc3c2)N(C(=O)C(=O)Nc2cncc3cn[nH]c23)C1 ; C[C@@H]1CC[C@@H](c2ccc3sc(CCN(C)C)nc3c2)N(C(=O)C(=O)Nc2ccn3ccnc3c2)C1] 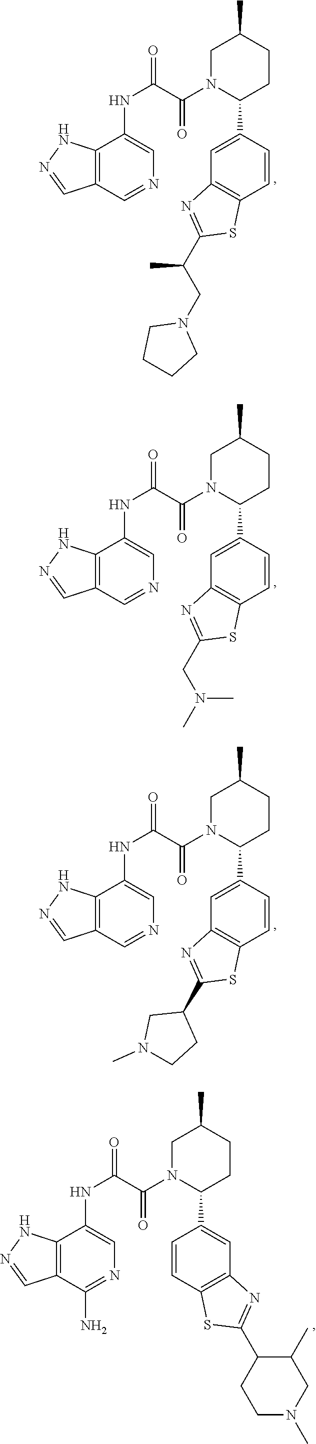 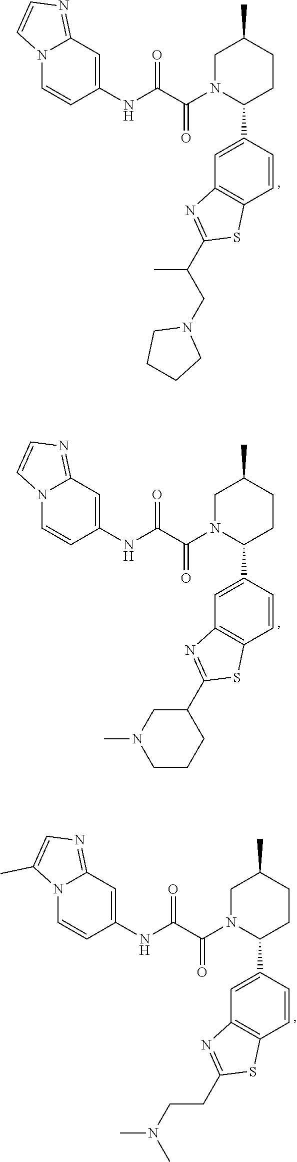 [SMILES: CC(CN1CCCC1)c1nc2cc([C@H]3CC[C@H](C)CN3C(=O)C(=O)Nc3ccn4ccnc4c3)ccc2s1 ; C[C@H]1CC[C@H](c2ccc3sc(C4CCCN(C)C4)nc3c2)N(C(=O)C(=O)Nc2ccn3ccnc3c2)C1 ; Cc1cnc2cc(NC(=O)C(=O)N3C[C@@H](C)CC[C@@H]3c3ccc4sc(CCN(C)C)nc4c3)ccn12] 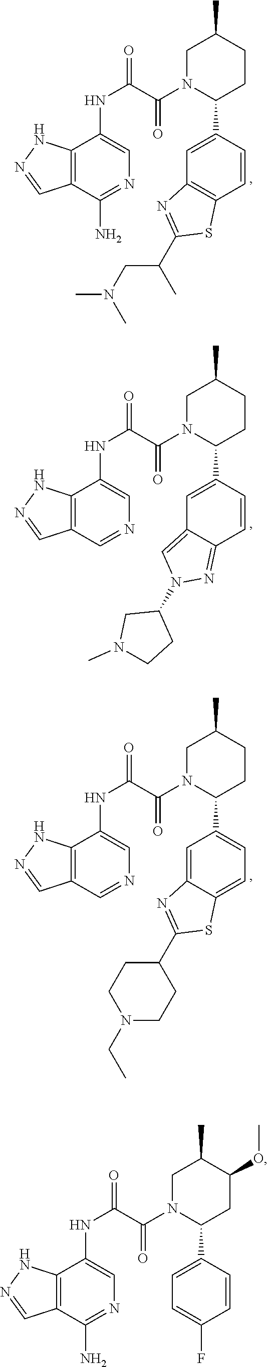 [SMILES: CC(CN(C)C)c1nc2cc([C@H]3CC[C@H](C)CN3C(=O)C(=O)Nc3cnc(N)c4cn[nH]c34)ccc2s1 ; C[C@H]1CC[C@H](c2ccc3nn([C@@H]4CCN(C)C4)cc3c2)N(C(=O)C(=O)Nc2cncc3cn[nH]c23)C1 ; CCN1CCC(c2nc3cc([C@H]4CC[C@H](C)CN4C(=O)C(=O)Nc4cncc5cn[nH]c45)ccc3s2)CC1 ; CO[C@H]1C[C@H](c2ccc(F)cc2)N(C(=O)C(=O)Nc2cnc(N)c3cn[nH]c23)C[C@H]1C] 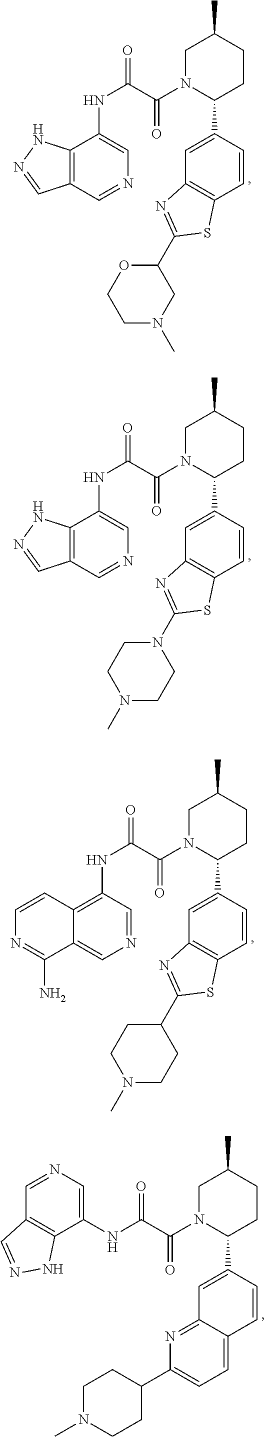 [SMILES: C[C@H]1CC[C@H](c2ccc3sc(C4CN(C)CCO4)nc3c2)N(C(=O)C(=O)Nc2cncc3cn[nH]c23)C1 ; C[C@H]1CC[C@H](c2ccc3sc(N4CCN(C)CC4)nc3c2)N(C(=O)C(=O)Nc2cncc3cn[nH]c23)C1 ; C[C@H]1CC[C@H](c2ccc3sc(C4CCN(C)CC4)nc3c2)N(C(=O)C(=O)Nc2cncc3c(N)nccc23)C1 ; C[C@H]1CC[C@H](c2ccc3ccc(C4CCN(C)CC4)nc3c2)N(C(=O)C(=O)Nc2cncc3cn[nH]c23)C1] 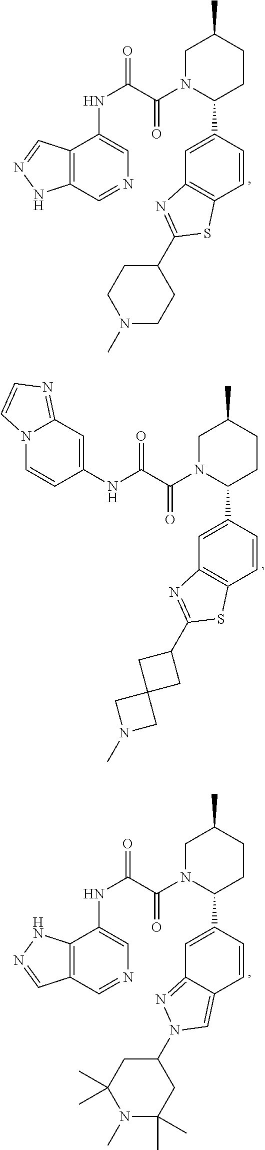 [SMILES: C[C@H]1CC[C@H](c2ccc3sc(C4CCN(C)CC4)nc3c2)N(C(=O)C(=O)Nc2cncc3[nH]ncc23)C1 ; C[C@H]1CC[C@H](c2ccc3sc(C4CC5(C4)CN(C)C5)nc3c2)N(C(=O)C(=O)Nc2ccn3ccnc3c2)C1 ; C[C@H]1CC[C@H](c2ccc3cn(C4CC(C)(C)N(C)C(C)(C)C4)nc3c2)N(C(=O)C(=O)Nc2cncc3cn[nH]c23)C1] 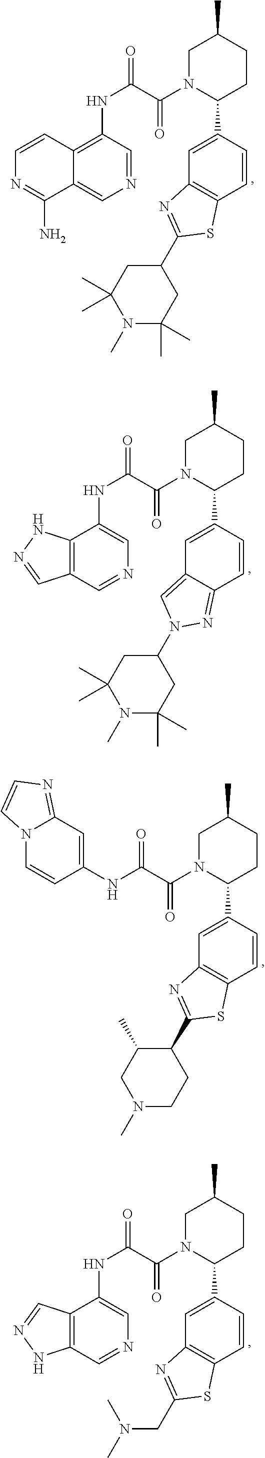 [SMILES: C[C@H]1CC[C@H](c2ccc3sc(C4CC(C)(C)N(C)C(C)(C)C4)nc3c2)N(C(=O)C(=O)Nc2cncc3c(N)nccc23)C1 ; C[C@H]1CC[C@H](c2ccc3nn(C4CC(C)(C)N(C)C(C)(C)C4)cc3c2)N(C(=O)C(=O)Nc2cncc3cn[nH]c23)C1 ; C[C@H]1CC[C@H](c2ccc3sc([C@H]4CCN(C)C[C@@H]4C)nc3c2)N(C(=O)C(=O)Nc2ccn3ccnc3c2)C1 ; C[C@H]1CC[C@H](c2ccc3sc(CN(C)C)nc3c2)N(C(=O)C(=O)Nc2cncc3[nH]ncc23)C1] 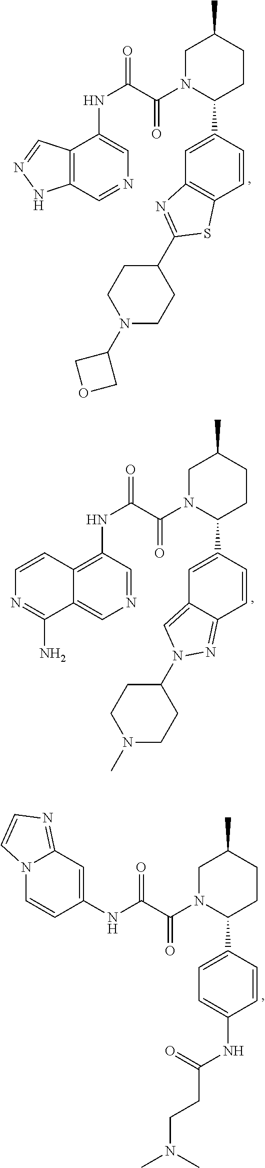 [SMILES: C[C@H]1CC[C@H](c2ccc3sc(C4CCN(C5COC5)CC4)nc3c2)N(C(=O)C(=O)Nc2cncc3[nH]ncc23)C1 ; C[C@H]1CC[C@H](c2ccc3nn(C4CCN(C)CC4)cc3c2)N(C(=O)C(=O)Nc2cncc3c(N)nccc23)C1 ; C[C@H]1CC[C@H](c2ccc(NC(=O)CCN(C)C)cc2)N(C(=O)C(=O)Nc2ccn3ccnc3c2)C1] 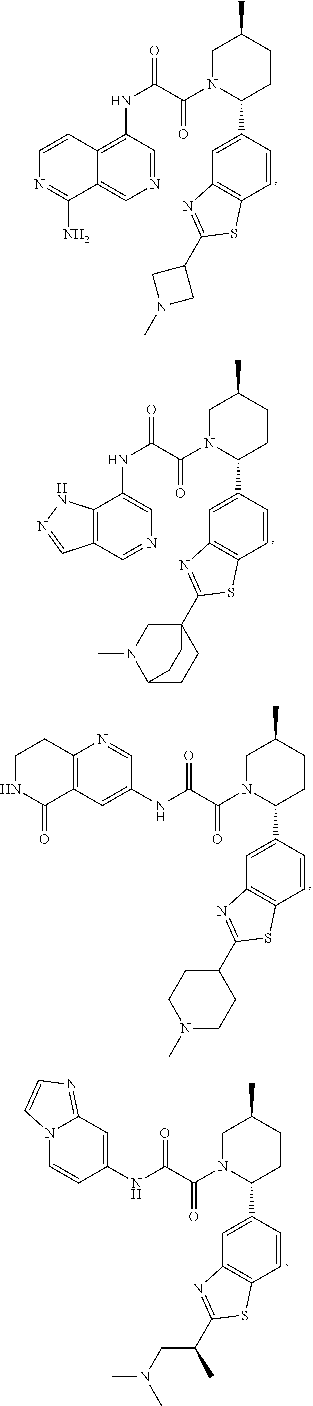 [SMILES: C[C@H]1CC[C@H](c2ccc3sc(C4CN(C)C4)nc3c2)N(C(=O)C(=O)Nc2cncc3c(N)nccc23)C1 ; C[C@H]1CC[C@H](c2ccc3sc(C45CCC(CC4)N(C)C5)nc3c2)N(C(=O)C(=O)Nc2cncc3cn[nH]c23)C1 ; C[C@H]1CC[C@H](c2ccc3sc(C4CCN(C)CC4)nc3c2)N(C(=O)C(=O)Nc2cnc3c(c2)C(=O)NCC3)C1 ; C[C@H]1CC[C@H](c2ccc3sc([C@@H](C)CN(C)C)nc3c2)N(C(=O)C(=O)Nc2ccn3ccnc3c2)C1] 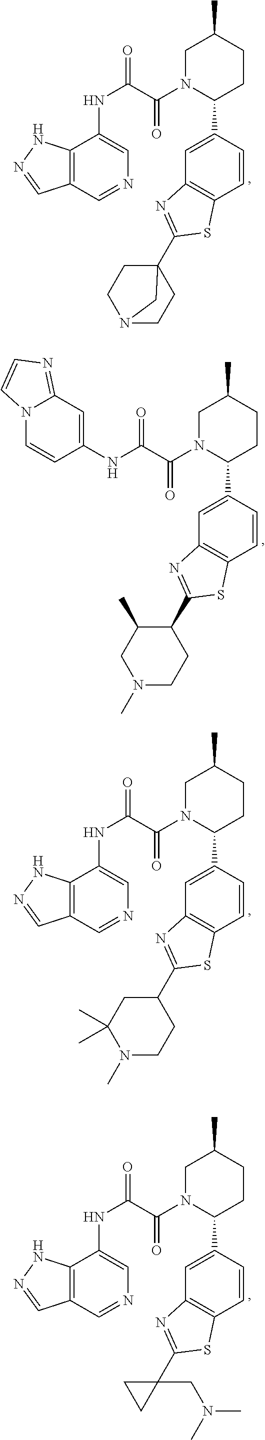 [SMILES: C[C@H]1CC[C@H](c2ccc3sc(C45CCN(CC4)C5)nc3c2)N(C(=O)C(=O)Nc2cncc3cn[nH]c23)C1 ; C[C@H]1CC[C@H](c2ccc3sc([C@H]4CCN(C)C[C@H]4C)nc3c2)N(C(=O)C(=O)Nc2ccn3ccnc3c2)C1 ; C[C@H]1CC[C@H](c2ccc3sc(C4CCN(C)C(C)(C)C4)nc3c2)N(C(=O)C(=O)Nc2cncc3cn[nH]c23)C1 ; C[C@H]1CC[C@H](c2ccc3sc(C4(CN(C)C)CC4)nc3c2)N(C(=O)C(=O)Nc2cncc3cn[nH]c23)C1] 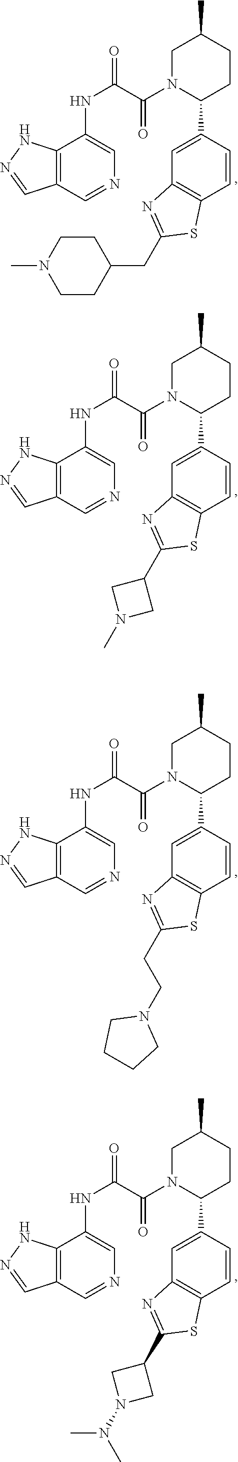 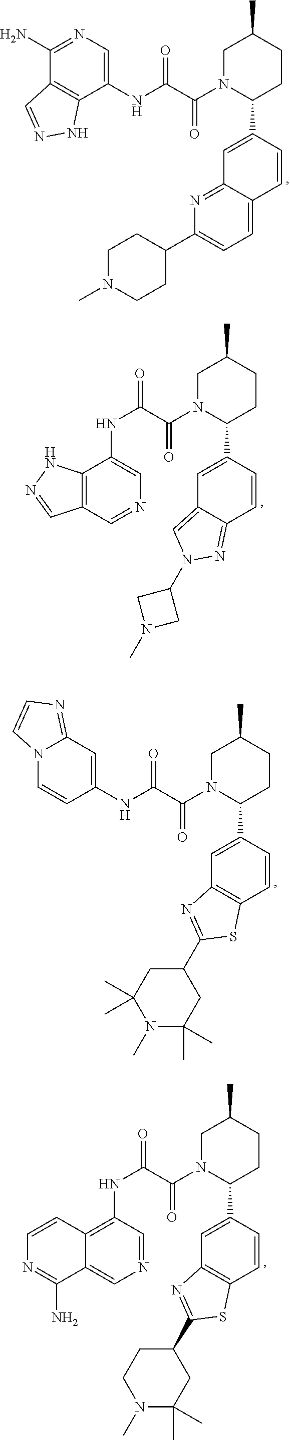 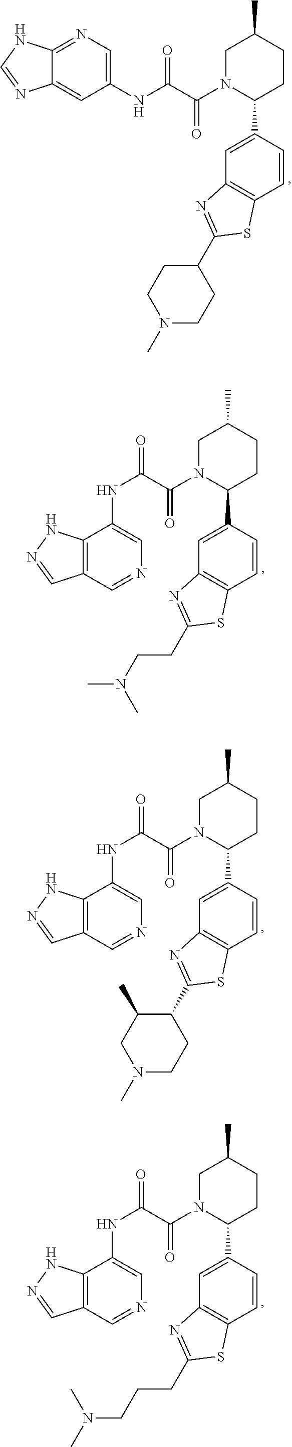 [SMILES: C[C@H]1CC[C@H](c2ccc3sc(C4CCN(C)CC4)nc3c2)N(C(=O)C(=O)Nc2cnc3[nH]cnc3c2)C1 ; C[C@@H]1CC[C@@H](c2ccc3sc(CCN(C)C)nc3c2)N(C(=O)C(=O)Nc2cncc3cn[nH]c23)C1 ; C[C@H]1CC[C@H](c2ccc3sc([C@@H]4CCN(C)C[C@H]4C)nc3c2)N(C(=O)C(=O)Nc2cncc3cn[nH]c23)C1 ; C[C@H]1CC[C@H](c2ccc3sc(CCCN(C)C)nc3c2)N(C(=O)C(=O)Nc2cncc3cn[nH]c23)C1] 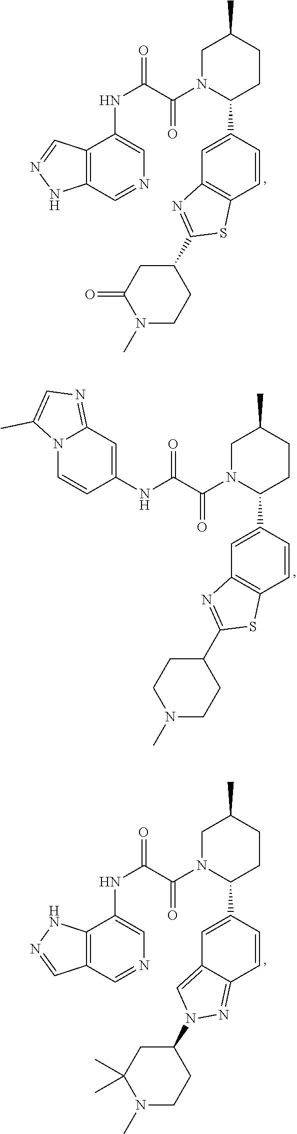 [SMILES: C[C@H]1CC[C@H](c2ccc3sc([C@@H]4CCN(C)C(=O)C4)nc3c2)N(C(=O)C(=O)Nc2cncc3[nH]ncc23)C1 ; Cc1cnc2cc(NC(=O)C(=O)N3C[C@@H](C)CC[C@@H]3c3ccc4sc(C5CCN(C)CC5)nc4c3)ccn12 ; C[C@H]1CC[C@H](c2ccc3nn([C@H]4CCN(C)C(C)(C)C4)cc3c2)N(C(=O)C(=O)Nc2cncc3cn[nH]c23)C1] 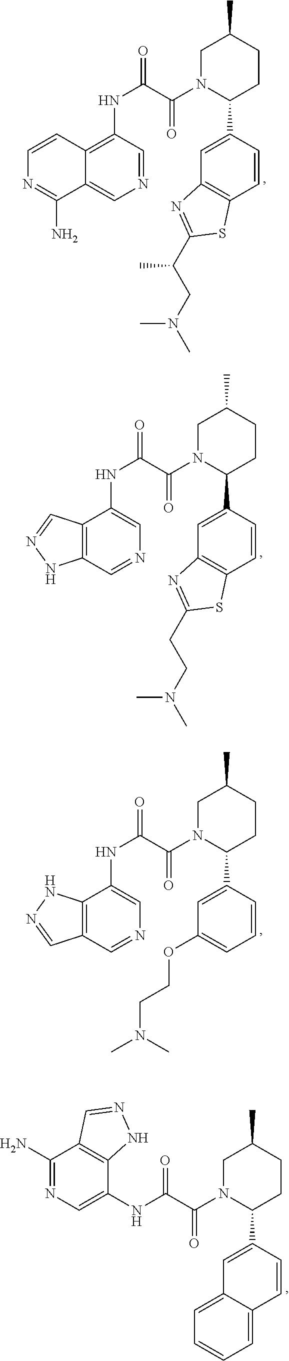 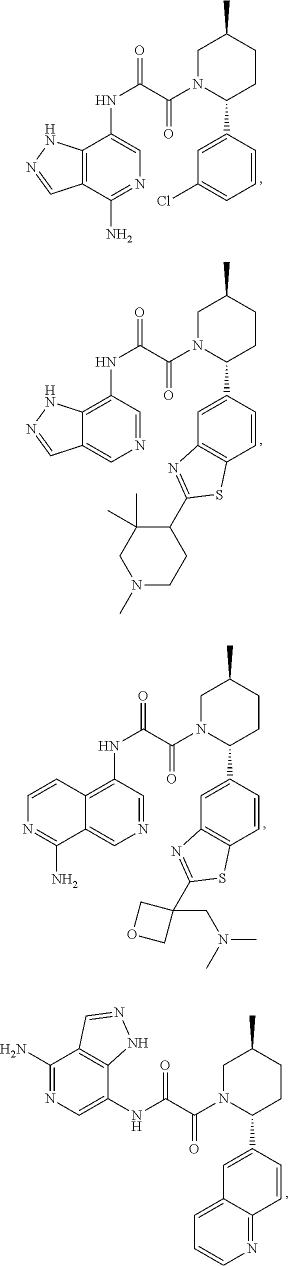 [SMILES: C[C@H]1CC[C@H](c2cccc(Cl)c2)N(C(=O)C(=O)Nc2cnc(N)c3cn[nH]c23)C1 ; C[C@H]1CC[C@H](c2ccc3sc(C4CCN(C)CC4(C)C)nc3c2)N(C(=O)C(=O)Nc2cncc3cn[nH]c23)C1 ; C[C@H]1CC[C@H](c2ccc3sc(C4(CN(C)C)COC4)nc3c2)N(C(=O)C(=O)Nc2cncc3c(N)nccc23)C1 ; C[C@H]1CC[C@H](c2ccc3ncccc3c2)N(C(=O)C(=O)Nc2cnc(N)c3cn[nH]c23)C1] 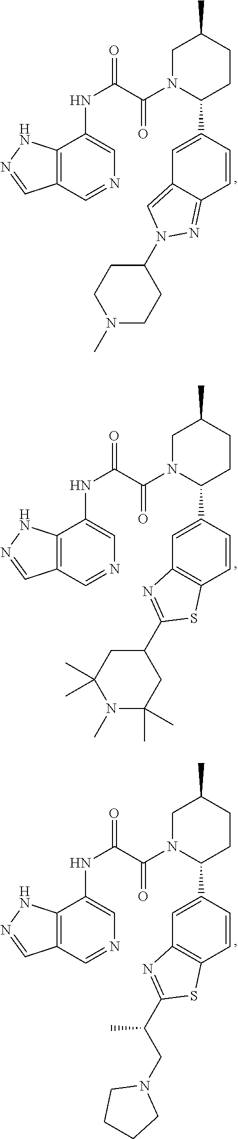 [SMILES: C[C@H]1CC[C@H](c2ccc3nn(C4CCN(C)CC4)cc3c2)N(C(=O)C(=O)Nc2cncc3cn[nH]c23)C1 ; C[C@H]1CC[C@H](c2ccc3sc(C4CC(C)(C)N(C)C(C)(C)C4)nc3c2)N(C(=O)C(=O)Nc2cncc3cn[nH]c23)C1 ; C[C@H]1CC[C@H](c2ccc3sc([C@@H](C)CN4CCCC4)nc3c2)N(C(=O)C(=O)Nc2cncc3cn[nH]c23)C1] 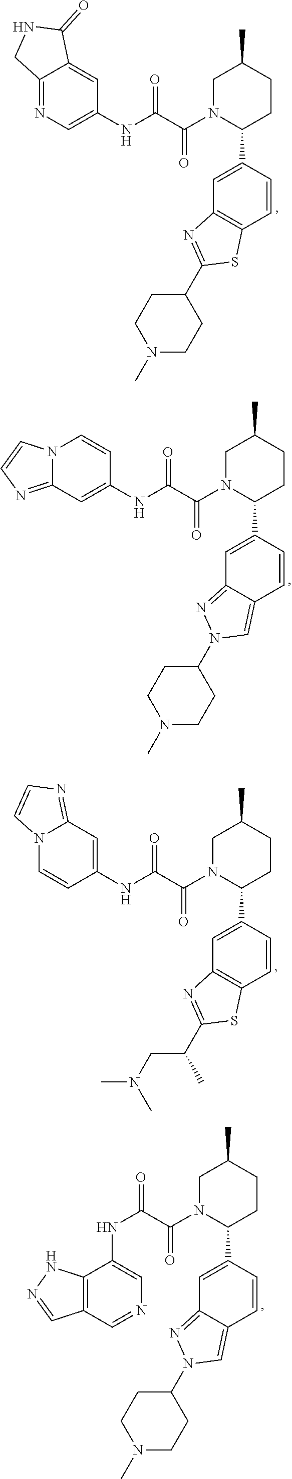 [SMILES: C[C@H]1CC[C@H](c2ccc3sc(C4CCN(C)CC4)nc3c2)N(C(=O)C(=O)Nc2cnc3c(c2)C(=O)NC3)C1 ; C[C@H]1CC[C@H](c2ccc3cn(C4CCN(C)CC4)nc3c2)N(C(=O)C(=O)Nc2ccn3ccnc3c2)C1 ; C[C@H]1CC[C@H](c2ccc3sc([C@H](C)CN(C)C)nc3c2)N(C(=O)C(=O)Nc2ccn3ccnc3c2)C1 ; C[C@H]1CC[C@H](c2ccc3cn(C4CCN(C)CC4)nc3c2)N(C(=O)C(=O)Nc2cncc3cn[nH]c23)C1] 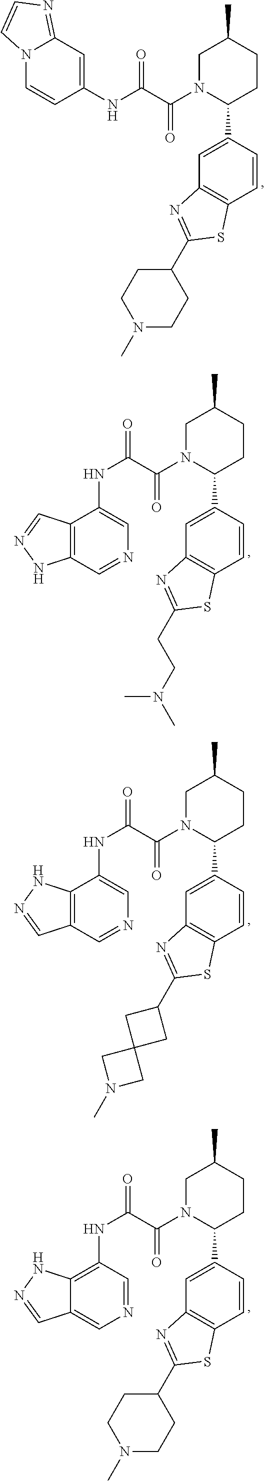 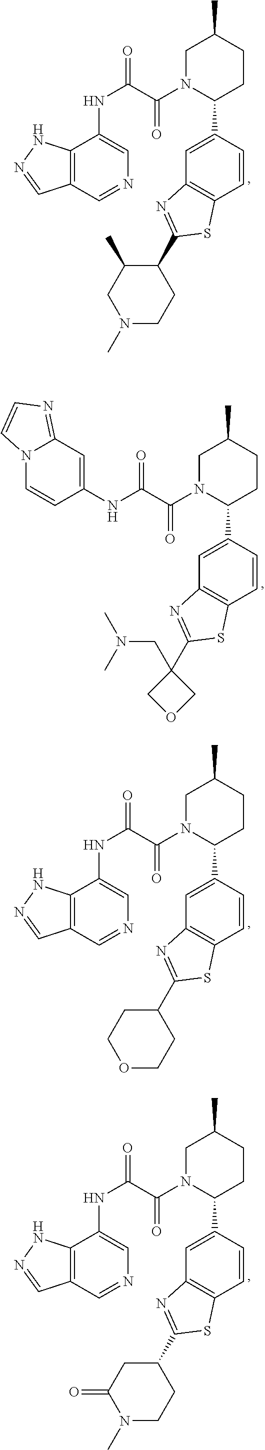 [SMILES: C[C@H]1CC[C@H](c2ccc3sc([C@H]4CCN(C)C[C@H]4C)nc3c2)N(C(=O)C(=O)Nc2cncc3cn[nH]c23)C1 ; C[C@H]1CC[C@H](c2ccc3sc(C4(CN(C)C)COC4)nc3c2)N(C(=O)C(=O)Nc2ccn3ccnc3c2)C1 ; C[C@H]1CC[C@H](c2ccc3sc(C4CCOCC4)nc3c2)N(C(=O)C(=O)Nc2cncc3cn[nH]c23)C1 ; C[C@H]1CC[C@H](c2ccc3sc([C@@H]4CCN(C)C(=O)C4)nc3c2)N(C(=O)C(=O)Nc2cncc3cn[nH]c23)C1] 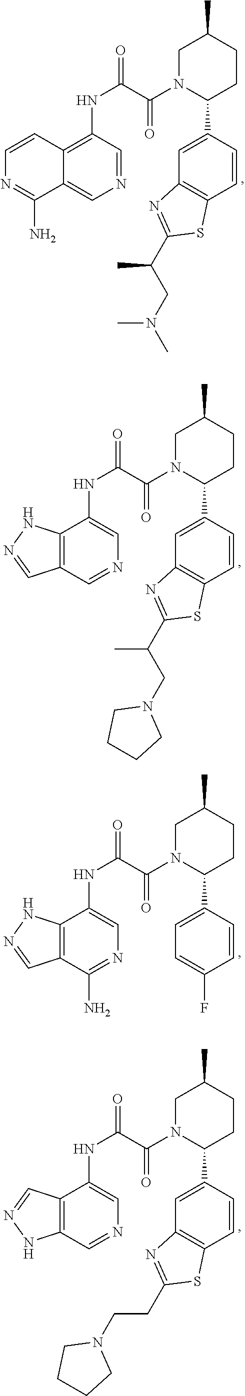 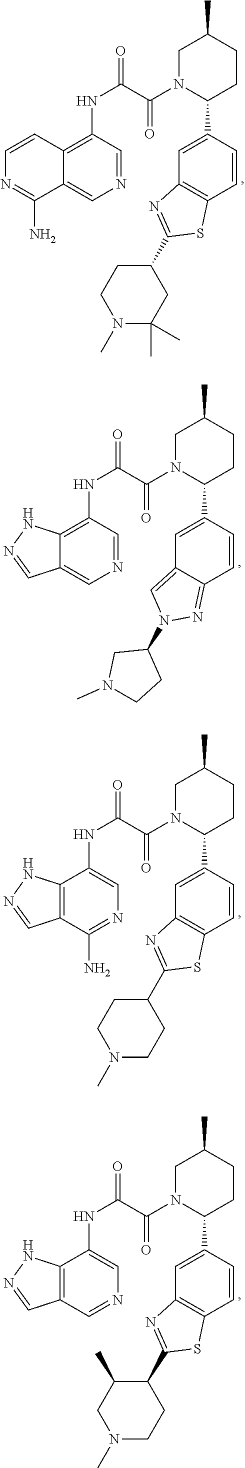 [SMILES: C[C@H]1CC[C@H](c2ccc3sc([C@H]4CCN(C)C(C)(C)C4)nc3c2)N(C(=O)C(=O)Nc2cncc3c(N)nccc23)C1 ; C[C@H]1CC[C@H](c2ccc3nn([C@H]4CCN(C)C4)cc3c2)N(C(=O)C(=O)Nc2cncc3cn[nH]c23)C1 ; C[C@H]1CC[C@H](c2ccc3sc(C4CCN(C)CC4)nc3c2)N(C(=O)C(=O)Nc2cnc(N)c3cn[nH]c23)C1 ; C[C@H]1CC[C@H](c2ccc3sc([C@H]4CCN(C)C[C@H]4C)nc3c2)N(C(=O)C(=O)Nc2cncc3cn[nH]c23)C1] 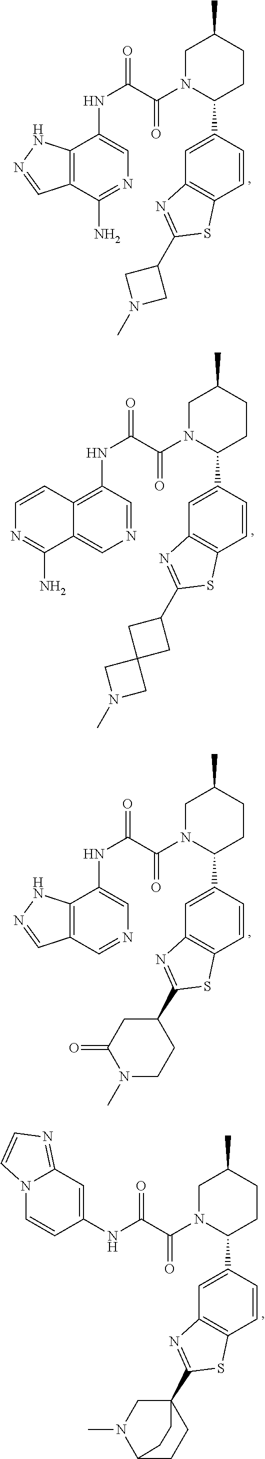 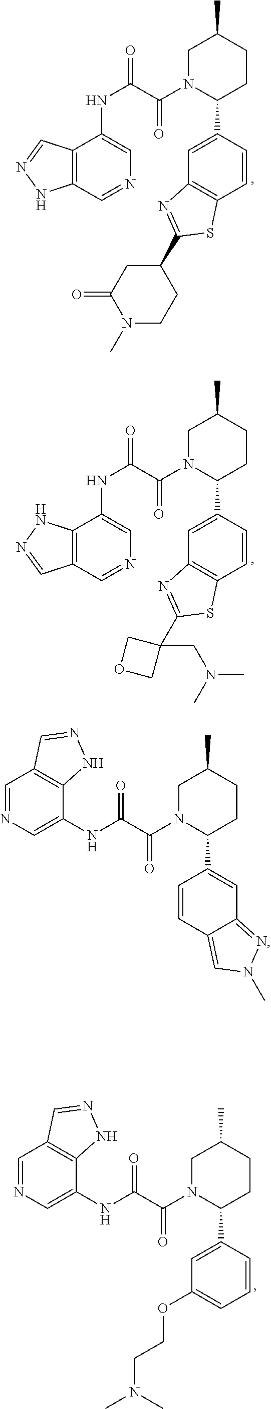 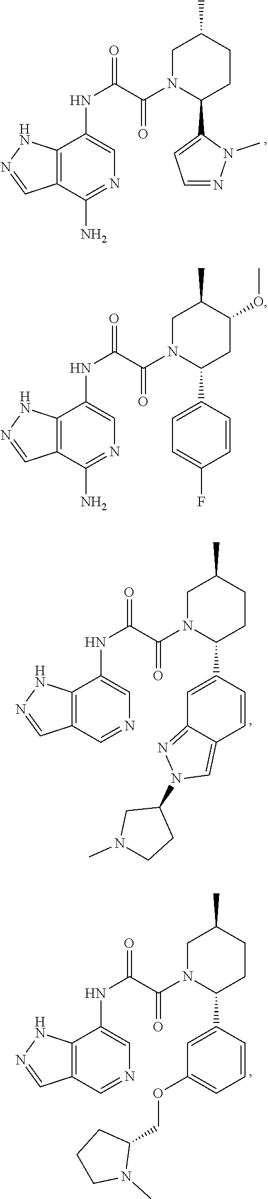 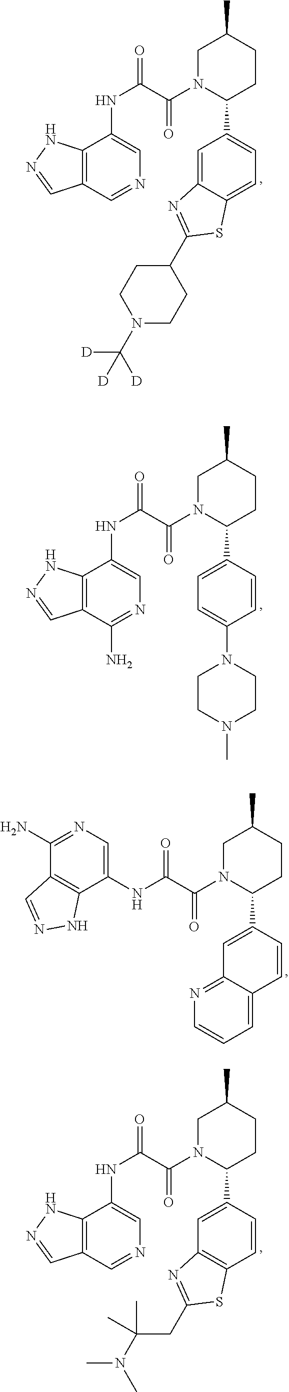 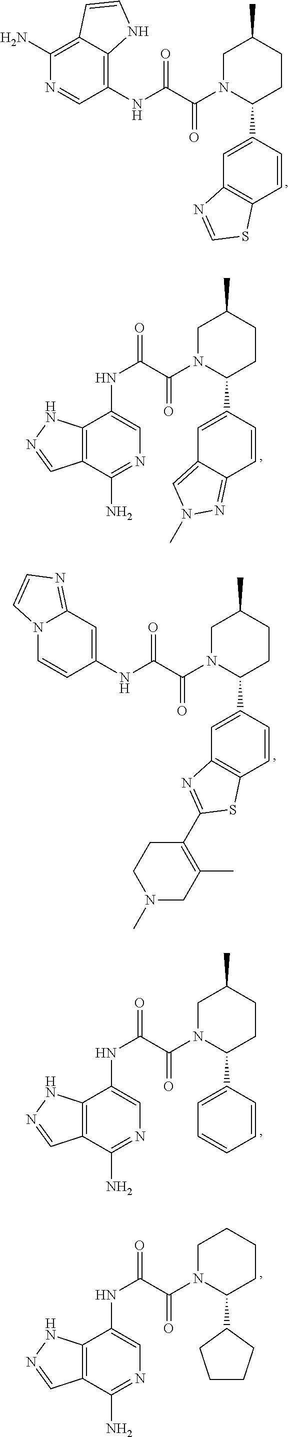 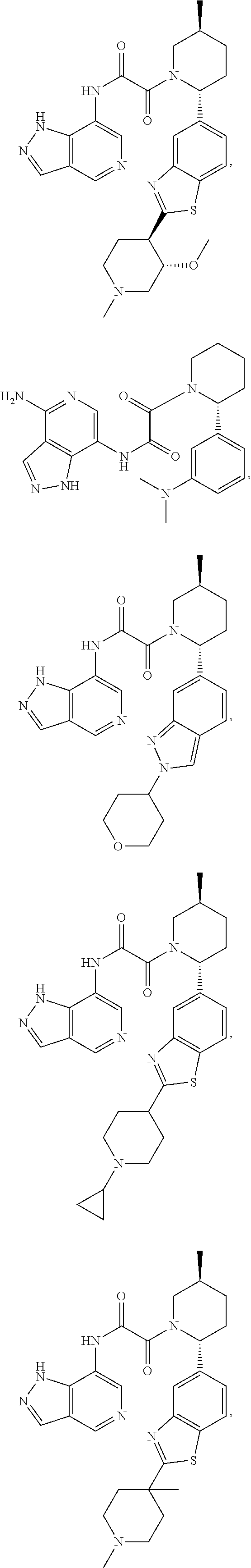 [SMILES: CO[C@@H]1CN(C)CC[C@H]1c1nc2cc([C@H]3CC[C@H](C)CN3C(=O)C(=O)Nc3cncc4cn[nH]c34)ccc2s1 ; CN(C)c1cccc([C@H]2CCCCN2C(=O)C(=O)Nc2cnc(N)c3cn[nH]c23)c1 ; C[C@H]1CC[C@H](c2ccc3cn(C4CCOCC4)nc3c2)N(C(=O)C(=O)Nc2cncc3cn[nH]c23)C1 ; C[C@H]1CC[C@H](c2ccc3sc(C4CCN(C5CC5)CC4)nc3c2)N(C(=O)C(=O)Nc2cncc3cn[nH]c23)C1 ; C[C@H]1CC[C@H](c2ccc3sc(C4(C)CCN(C)CC4)nc3c2)N(C(=O)C(=O)Nc2cncc3cn[nH]c23)C1] 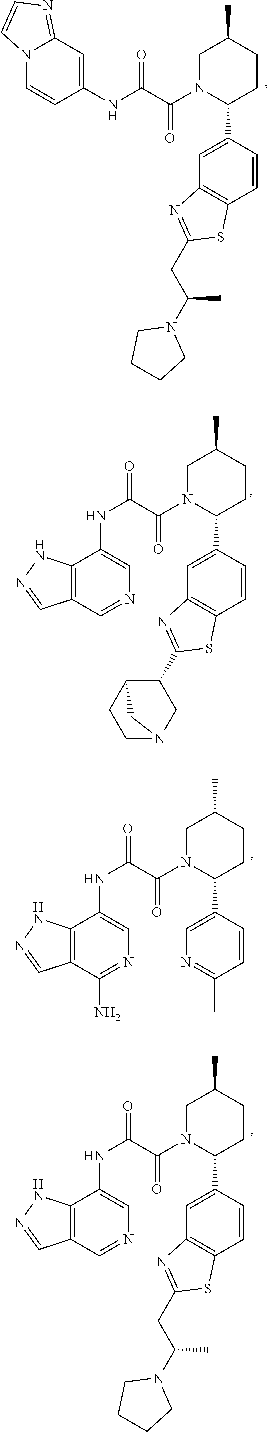 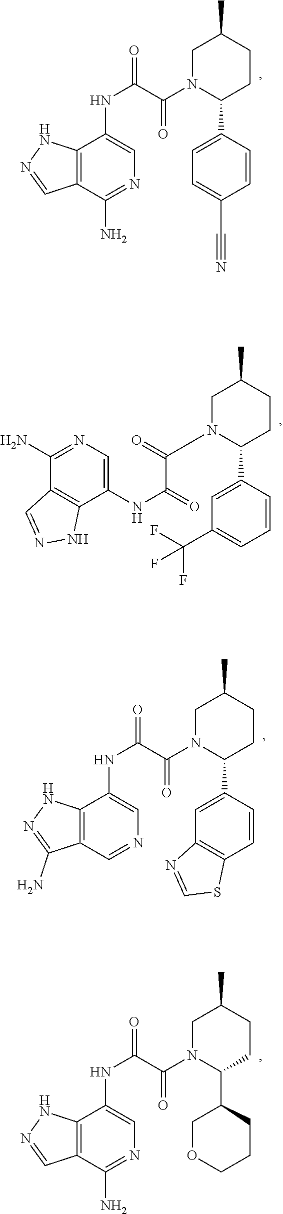 [SMILES: C[C@H]1CC[C@H](c2ccc(C#N)cc2)N(C(=O)C(=O)Nc2cnc(N)c3cn[nH]c23)C1 ; C[C@H]1CC[C@H](c2cccc(C(F)(F)F)c2)N(C(=O)C(=O)Nc2cnc(N)c3cn[nH]c23)C1 ; C[C@H]1CC[C@H](c2ccc3scnc3c2)N(C(=O)C(=O)Nc2cncc3c(N)n[nH]c23)C1 ; C[C@H]1CC[C@H]([C@H]2CCCOC2)N(C(=O)C(=O)Nc2cnc(N)c3cn[nH]c23)C1] 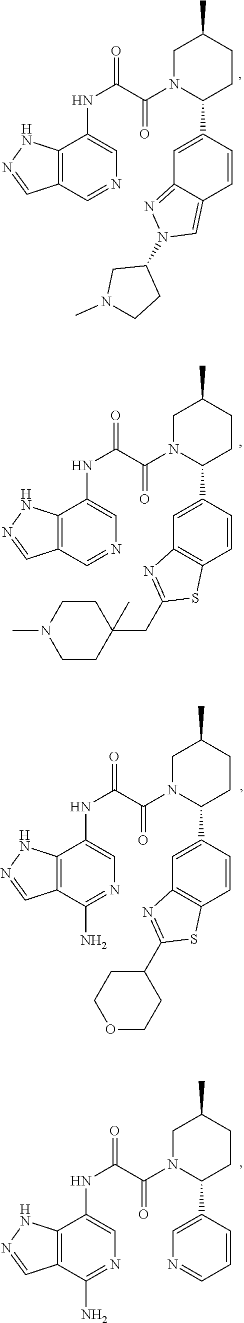 [SMILES: C[C@H]1CC[C@H](c2ccc3cn([C@@H]4CCN(C)C4)nc3c2)N(C(=O)C(=O)Nc2cncc3cn[nH]c23)C1 ; C[C@H]1CC[C@H](c2ccc3sc(CC4(C)CCN(C)CC4)nc3c2)N(C(=O)C(=O)Nc2cncc3cn[nH]c23)C1 ; C[C@H]1CC[C@H](c2ccc3sc(C4CCOCC4)nc3c2)N(C(=O)C(=O)Nc2cnc(N)c3cn[nH]c23)C1 ; C[C@H]1CC[C@H](c2cccnc2)N(C(=O)C(=O)Nc2cnc(N)c3cn[nH]c23)C1] 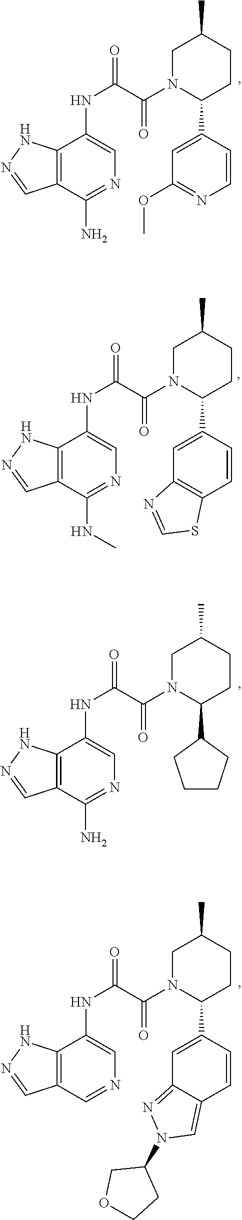 [SMILES: COc1cc([C@H]2CC[C@H](C)CN2C(=O)C(=O)Nc2cnc(N)c3cn[nH]c23)ccn1 ; CNc1ncc(NC(=O)C(=O)N2C[C@@H](C)CC[C@@H]2c2ccc3scnc3c2)c2[nH]ncc12 ; C[C@@H]1CC[C@@H](C2CCCC2)N(C(=O)C(=O)Nc2cnc(N)c3cn[nH]c23)C1 ; C[C@H]1CC[C@H](c2ccc3cn([C@H]4CCOC4)nc3c2)N(C(=O)C(=O)Nc2cncc3cn[nH]c23)C1] 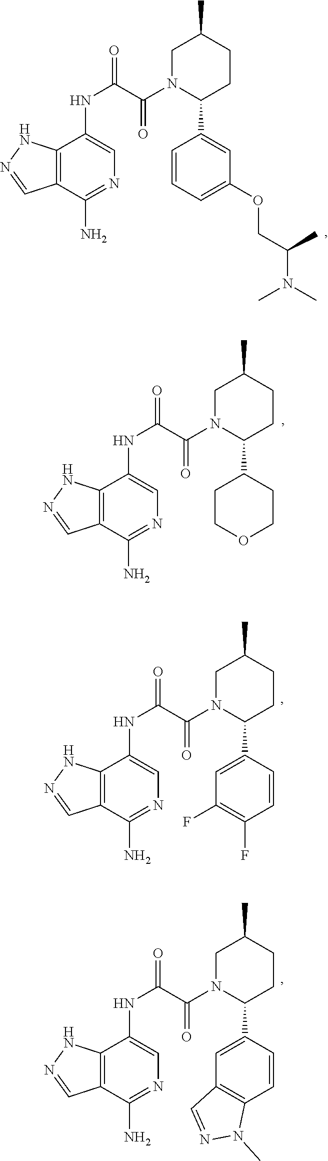 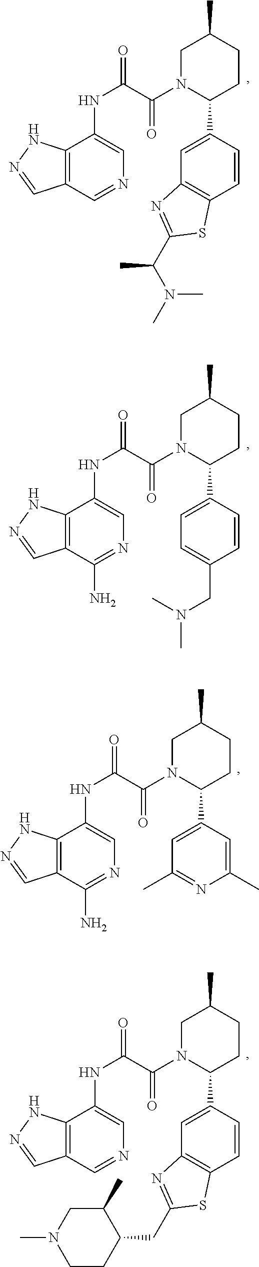 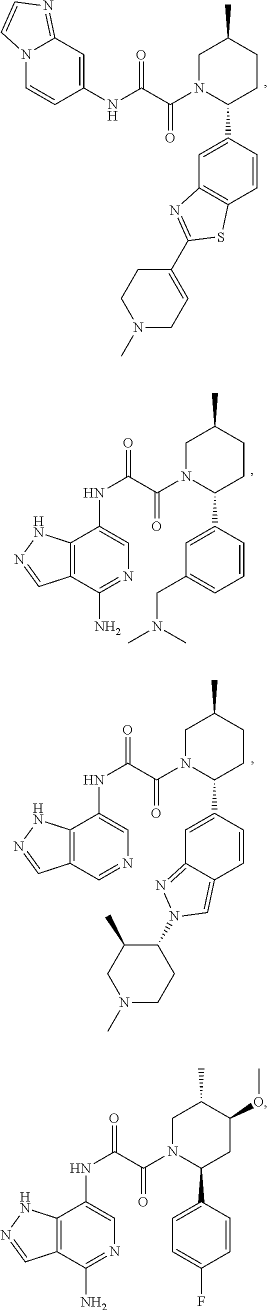 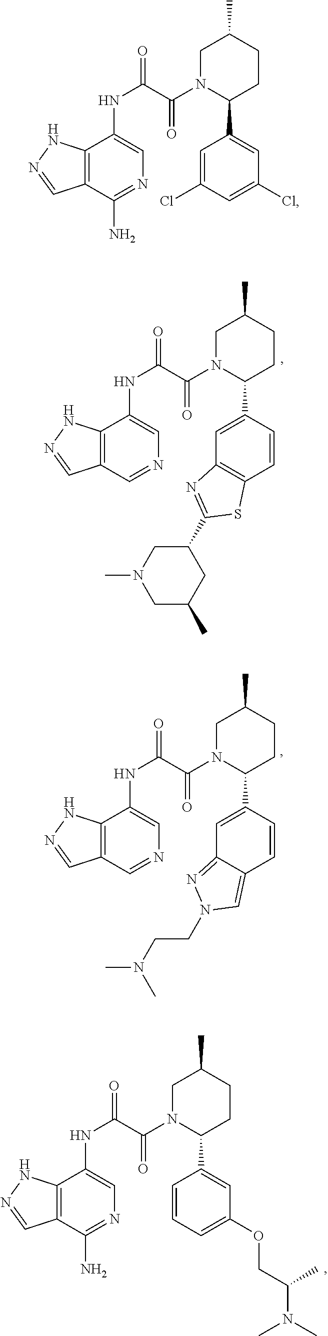 [SMILES: C[C@@H]1CC[C@@H](c2cc(Cl)cc(Cl)c2)N(C(=O)C(=O)Nc2cnc(N)c3cn[nH]c23)C1 ; C[C@@H]1C[C@@H](c2nc3cc([C@H]4CC[C@H](C)CN4C(=O)C(=O)Nc4cncc5cn[nH]c45)ccc3s2)CN(C)C1 ; C[C@H]1CC[C@H](c2ccc3cn(CCN(C)C)nc3c2)N(C(=O)C(=O)Nc2cncc3cn[nH]c23)C1 ; C[C@H]1CC[C@H](c2cccc(OC[C@H](C)N(C)C)c2)N(C(=O)C(=O)Nc2cnc(N)c3cn[nH]c23)C1] 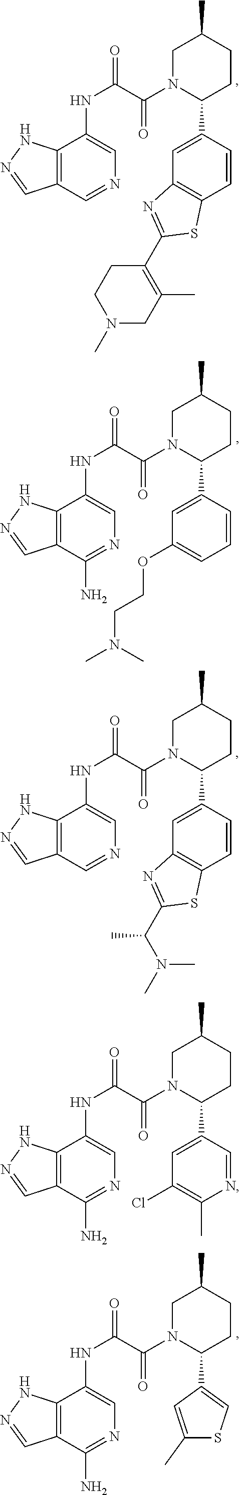 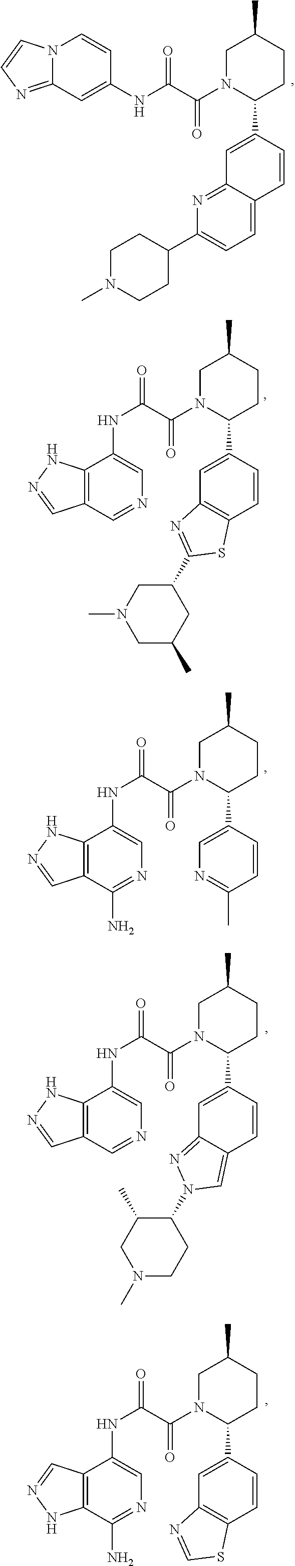 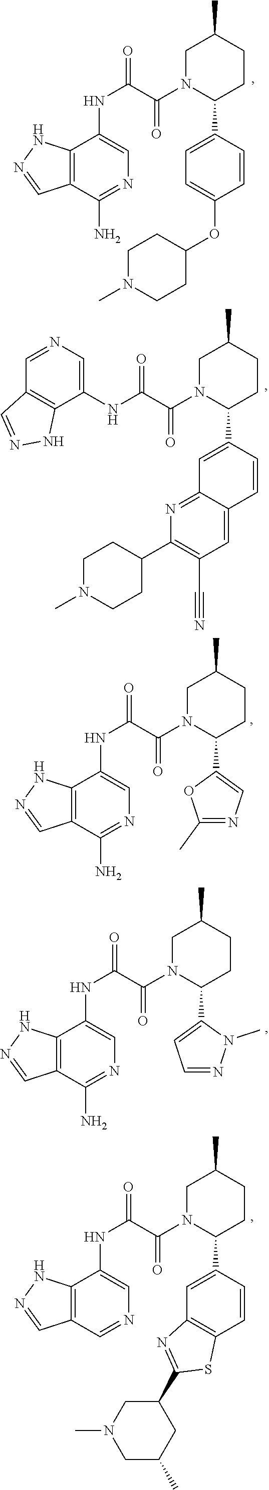 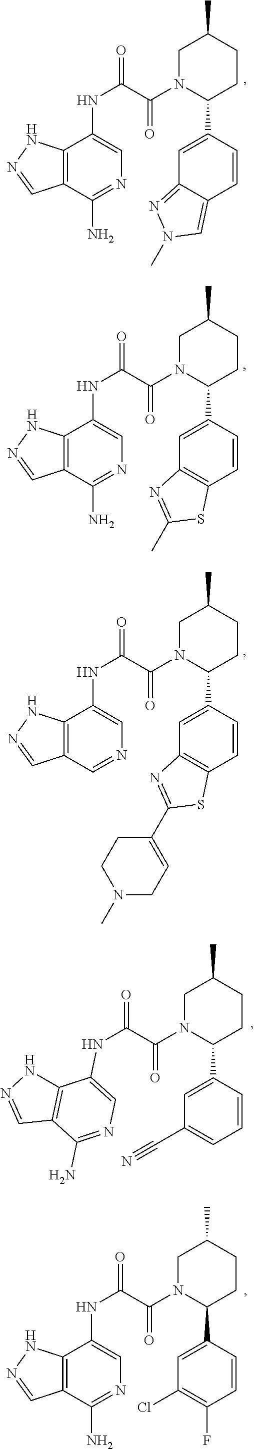 [SMILES: C[C@H]1CC[C@H](c2ccc3cn(C)nc3c2)N(C(=O)C(=O)Nc2cnc(N)c3cn[nH]c23)C1 ; Cc1nc2cc([C@H]3CC[C@H](C)CN3C(=O)C(=O)Nc3cnc(N)c4cn[nH]c34)ccc2s1 ; C[C@H]1CC[C@H](c2ccc3sc(C4=CCN(C)CC4)nc3c2)N(C(=O)C(=O)Nc2cncc3cn[nH]c23)C1 ; C[C@H]1CC[C@H](c2cccc(C#N)c2)N(C(=O)C(=O)Nc2cnc(N)c3cn[nH]c23)C1 ; C[C@@H]1CC[C@@H](c2ccc(F)c(Cl)c2)N(C(=O)C(=O)Nc2cnc(N)c3cn[nH]c23)C1] 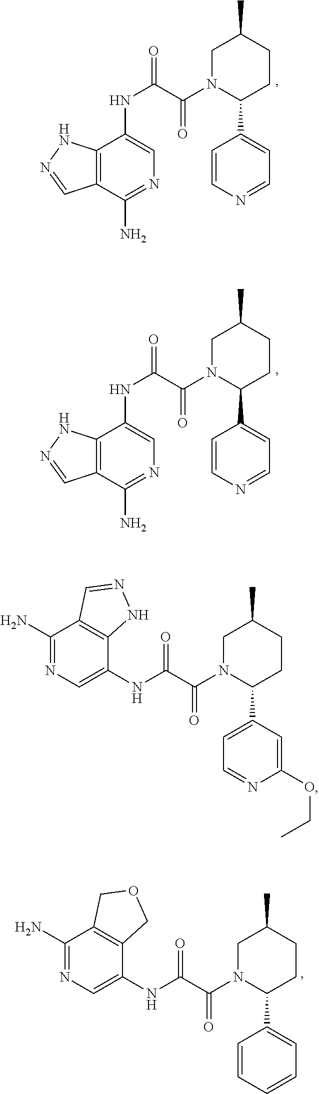 [SMILES: C[C@H]1CC[C@H](c2ccncc2)N(C(=O)C(=O)Nc2cnc(N)c3cn[nH]c23)C1 ; C[C@H]1CC[C@@H](c2ccncc2)N(C(=O)C(=O)Nc2cnc(N)c3cn[nH]c23)C1 ; CCOc1cc([C@H]2CC[C@H](C)CN2C(=O)C(=O)Nc2cnc(N)c3cn[nH]c23)ccn1 ; C[C@H]1CC[C@H](c2ccccc2)N(C(=O)C(=O)Nc2cnc(N)c3c2COC3)C1] 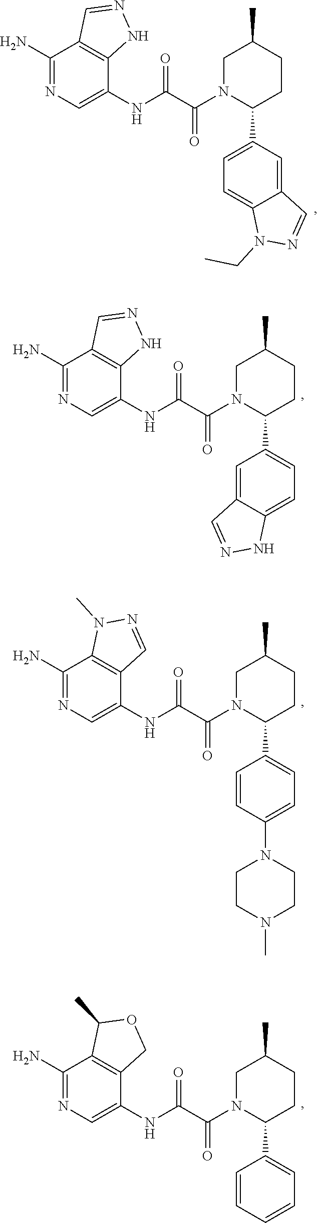 [SMILES: CCn1ncc2cc([C@H]3CC[C@H](C)CN3C(=O)C(=O)Nc3cnc(N)c4cn[nH]c34)ccc21 ; C[C@H]1CC[C@H](c2ccc3[nH]ncc3c2)N(C(=O)C(=O)Nc2cnc(N)c3cn[nH]c23)C1 ; C[C@H]1CC[C@H](c2ccc(N3CCN(C)CC3)cc2)N(C(=O)C(=O)Nc2cnc(N)c3c2cnn3C)C1 ; C[C@H]1CC[C@H](c2ccccc2)N(C(=O)C(=O)Nc2cnc(N)c3c2CO[C@@H]3C)C1] 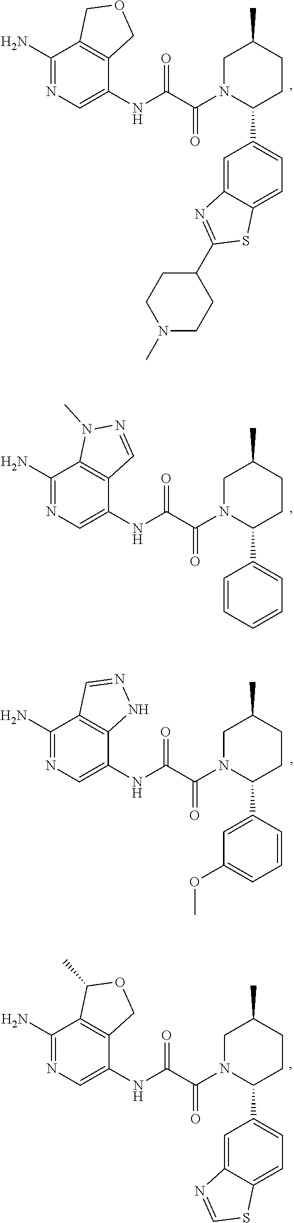 [SMILES: C[C@H]1CC[C@H](c2ccc3sc(C4CCN(C)CC4)nc3c2)N(C(=O)C(=O)Nc2cnc(N)c3c2COC3)C1 ; C[C@H]1CC[C@H](c2ccccc2)N(C(=O)C(=O)Nc2cnc(N)c3c2cnn3C)C1 ; COc1cccc([C@H]2CC[C@H](C)CN2C(=O)C(=O)Nc2cnc(N)c3cn[nH]c23)c1 ; C[C@H]1CC[C@H](c2ccc3scnc3c2)N(C(=O)C(=O)Nc2cnc(N)c3c2CO[C@H]3C)C1] 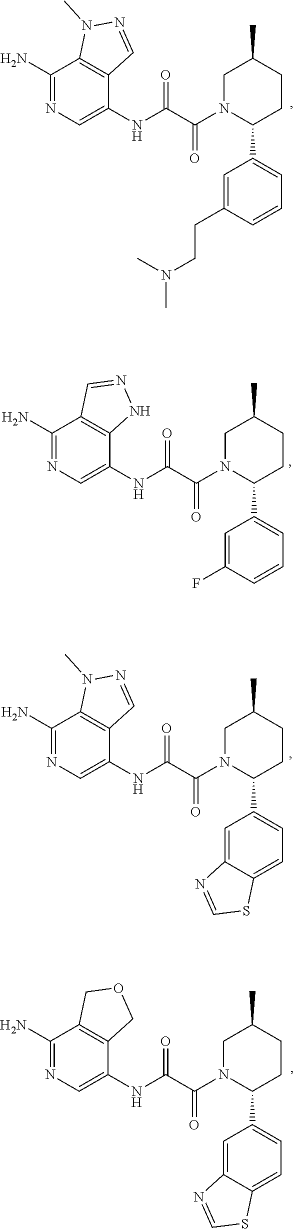 [SMILES: C[C@H]1CC[C@H](c2cccc(CCN(C)C)c2)N(C(=O)C(=O)Nc2cnc(N)c3c2cnn3C)C1 ; C[C@H]1CC[C@H](c2cccc(F)c2)N(C(=O)C(=O)Nc2cnc(N)c3cn[nH]c23)C1 ; C[C@H]1CC[C@H](c2ccc3scnc3c2)N(C(=O)C(=O)Nc2cnc(N)c3c2cnn3C)C1 ; C[C@H]1CC[C@H](c2ccc3scnc3c2)N(C(=O)C(=O)Nc2cnc(N)c3c2COC3)C1] 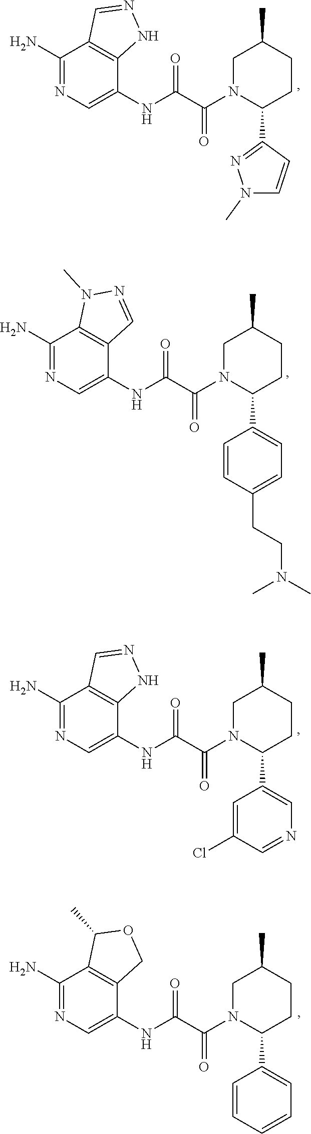 [SMILES: C[C@H]1CC[C@H](c2ccn(C)n2)N(C(=O)C(=O)Nc2cnc(N)c3cn[nH]c23)C1 ; C[C@H]1CC[C@H](c2ccc(CCN(C)C)cc2)N(C(=O)C(=O)Nc2cnc(N)c3c2cnn3C)C1 ; C[C@H]1CC[C@H](c2cncc(Cl)c2)N(C(=O)C(=O)Nc2cnc(N)c3cn[nH]c23)C1 ; C[C@H]1CC[C@H](c2ccccc2)N(C(=O)C(=O)Nc2cnc(N)c3c2CO[C@H]3C)C1] 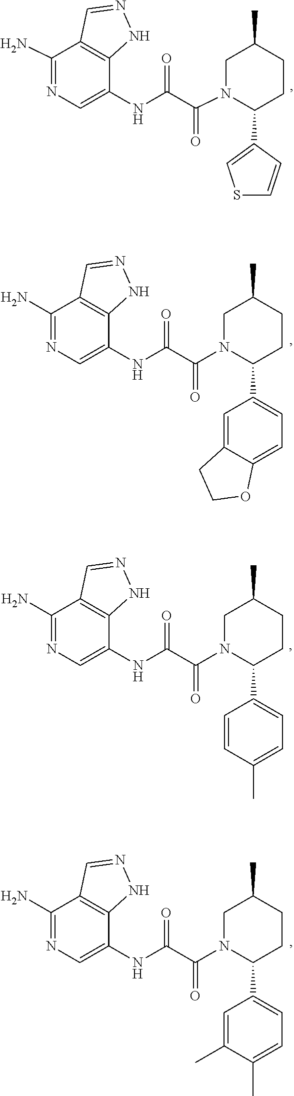 [SMILES: C[C@H]1CC[C@H](c2ccsc2)N(C(=O)C(=O)Nc2cnc(N)c3cn[nH]c23)C1 ; C[C@H]1CC[C@H](c2ccc3c(c2)CCO3)N(C(=O)C(=O)Nc2cnc(N)c3cn[nH]c23)C1 ; Cc1ccc([C@H]2CC[C@H](C)CN2C(=O)C(=O)Nc2cnc(N)c3cn[nH]c23)cc1 ; Cc1ccc([C@H]2CC[C@H](C)CN2C(=O)C(=O)Nc2cnc(N)c3cn[nH]c23)cc1C] 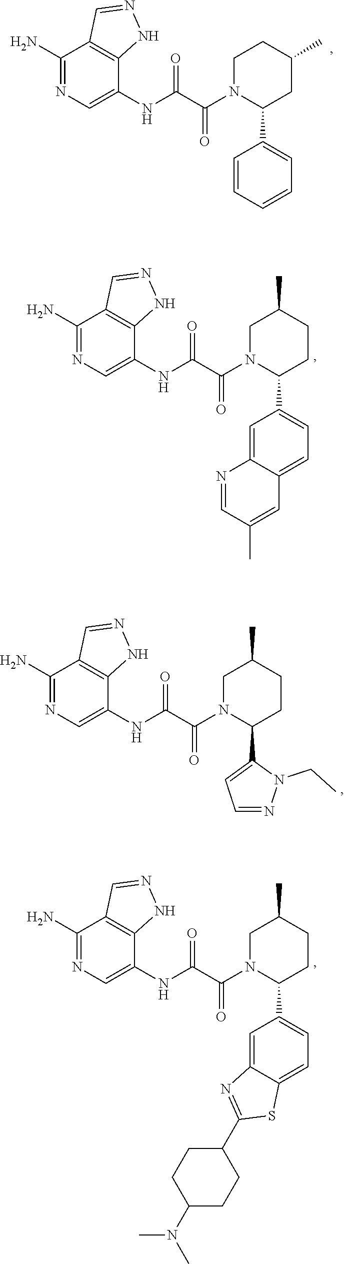 [SMILES: C[C@H]1CCN(C(=O)C(=O)Nc2cnc(N)c3cn[nH]c23)[C@@H](c2ccccc2)C1 ; Cc1cnc2cc([C@H]3CC[C@H](C)CN3C(=O)C(=O)Nc3cnc(N)c4cn[nH]c34)ccc2c1 ; CCn1nccc1[C@@H]1CC[C@H](C)CN1C(=O)C(=O)Nc1cnc(N)c2cn[nH]c12 ; C[C@H]1CC[C@H](c2ccc3sc(C4CCC(N(C)C)CC4)nc3c2)N(C(=O)C(=O)Nc2cnc(N)c3cn[nH]c23)C1] 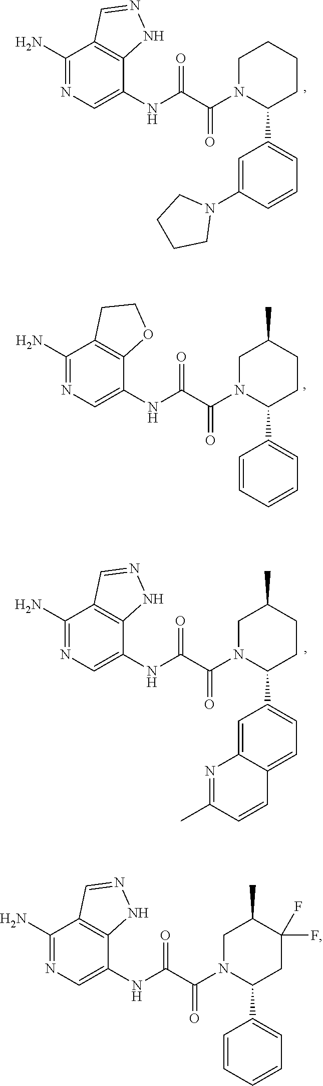 [SMILES: Nc1ncc(NC(=O)C(=O)N2CCCC[C@@H]2c2cccc(N3CCCC3)c2)c2[nH]ncc12 ; C[C@H]1CC[C@H](c2ccccc2)N(C(=O)C(=O)Nc2cnc(N)c3c2OCC3)C1 ; Cc1ccc2ccc([C@H]3CC[C@H](C)CN3C(=O)C(=O)Nc3cnc(N)c4cn[nH]c34)cc2n1 ; C[C@@H]1CN(C(=O)C(=O)Nc2cnc(N)c3cn[nH]c23)[C@@H](c2ccccc2)CC1(F)F] 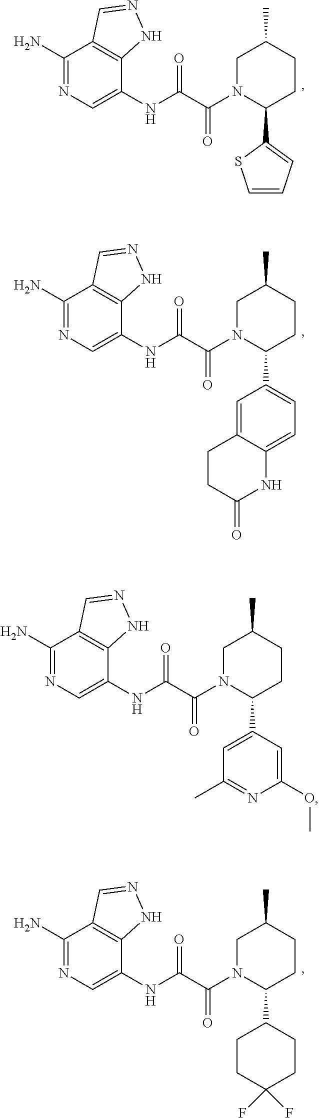 [SMILES: C[C@@H]1CC[C@@H](c2cccs2)N(C(=O)C(=O)Nc2cnc(N)c3cn[nH]c23)C1 ; C[C@H]1CC[C@H](c2ccc3c(c2)CCC(=O)N3)N(C(=O)C(=O)Nc2cnc(N)c3cn[nH]c23)C1 ; COc1cc([C@H]2CC[C@H](C)CN2C(=O)C(=O)Nc2cnc(N)c3cn[nH]c23)cc(C)n1 ; C[C@H]1CC[C@H](C2CCC(F)(F)CC2)N(C(=O)C(=O)Nc2cnc(N)c3cn[nH]c23)C1] 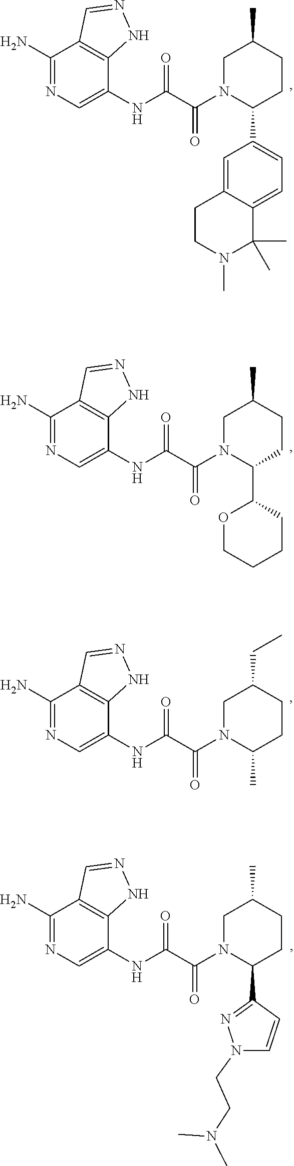 [SMILES: C[C@H]1CC[C@H](c2ccc3c(c2)CCN(C)C3(C)C)N(C(=O)C(=O)Nc2cnc(N)c3cn[nH]c23)C1 ; C[C@H]1CC[C@H]([C@@H]2CCCCO2)N(C(=O)C(=O)Nc2cnc(N)c3cn[nH]c23)C1 ; CC[C@@H]1CC[C@H](C)N(C(=O)C(=O)Nc2cnc(N)c3cn[nH]c23)C1 ; C[C@@H]1CC[C@@H](c2ccn(CCN(C)C)n2)N(C(=O)C(=O)Nc2cnc(N)c3cn[nH]c23)C1] 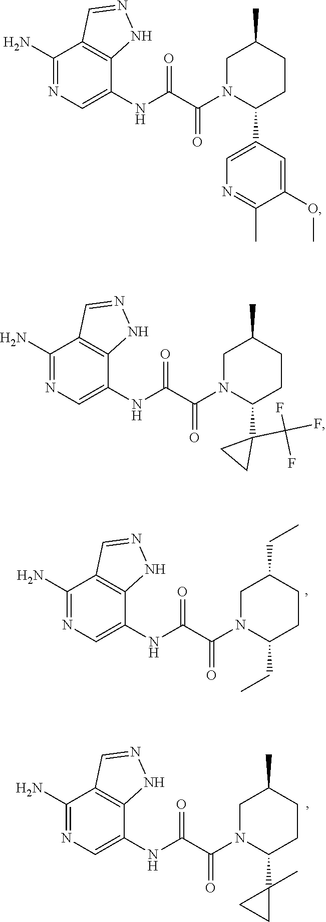 [SMILES: COc1cc([C@H]2CC[C@H](C)CN2C(=O)C(=O)Nc2cnc(N)c3cn[nH]c23)cnc1C ; C[C@H]1CC[C@H](C2(C(F)(F)F)CC2)N(C(=O)C(=O)Nc2cnc(N)c3cn[nH]c23)C1 ; CC[C@@H]1CC[C@H](CC)N(C(=O)C(=O)Nc2cnc(N)c3cn[nH]c23)C1 ; C[C@H]1CC[C@H](C2(C)CC2)N(C(=O)C(=O)Nc2cnc(N)c3cn[nH]c23)C1] 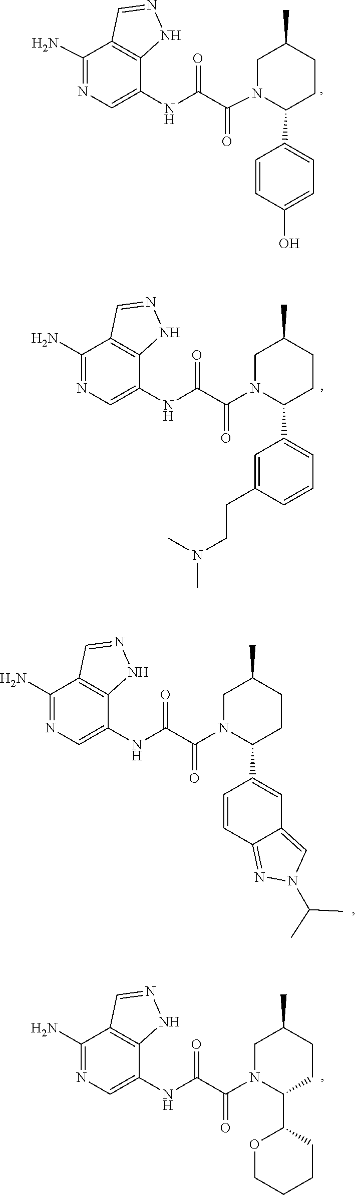 [SMILES: C[C@H]1CC[C@H](c2ccc(O)cc2)N(C(=O)C(=O)Nc2cnc(N)c3cn[nH]c23)C1 ; C[C@H]1CC[C@H](c2cccc(CCN(C)C)c2)N(C(=O)C(=O)Nc2cnc(N)c3cn[nH]c23)C1 ; CC(C)n1cc2cc([C@H]3CC[C@H](C)CN3C(=O)C(=O)Nc3cnc(N)c4cn[nH]c34)ccc2n1 ; C[C@H]1CC[C@H]([C@@H]2CCCCO2)N(C(=O)C(=O)Nc2cnc(N)c3cn[nH]c23)C1] 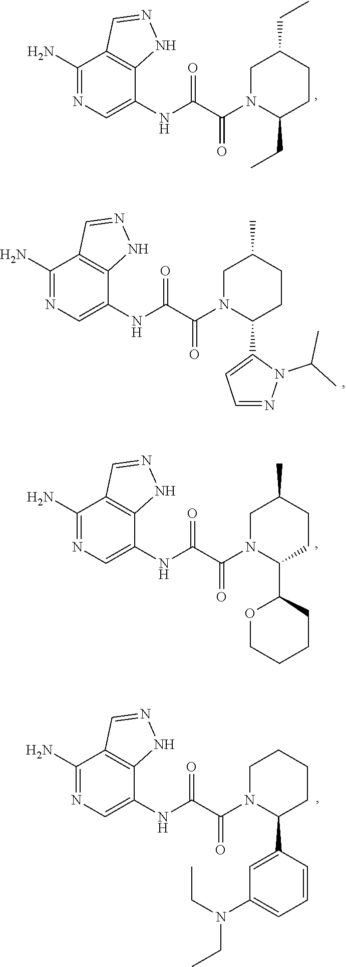 [SMILES: CC[C@@H]1CC[C@@H](CC)N(C(=O)C(=O)Nc2cnc(N)c3cn[nH]c23)C1 ; CC(C)n1nccc1[C@H]1CC[C@@H](C)CN1C(=O)C(=O)Nc1cnc(N)c2cn[nH]c12 ; C[C@H]1CC[C@H]([C@H]2CCCCO2)N(C(=O)C(=O)Nc2cnc(N)c3cn[nH]c23)C1 ; CCN(CC)c1cccc([C@@H]2CCCCN2C(=O)C(=O)Nc2cnc(N)c3cn[nH]c23)c1] 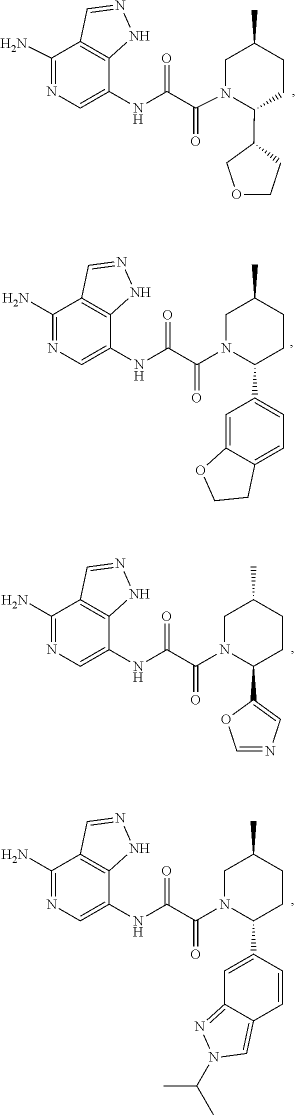 [SMILES: C[C@H]1CC[C@H]([C@@H]2CCOC2)N(C(=O)C(=O)Nc2cnc(N)c3cn[nH]c23)C1 ; C[C@H]1CC[C@H](c2ccc3c(c2)OCC3)N(C(=O)C(=O)Nc2cnc(N)c3cn[nH]c23)C1 ; C[C@@H]1CC[C@@H](c2cnco2)N(C(=O)C(=O)Nc2cnc(N)c3cn[nH]c23)C1 ; CC(C)n1cc2ccc([C@H]3CC[C@H](C)CN3C(=O)C(=O)Nc3cnc(N)c4cn[nH]c34)cc2n1] 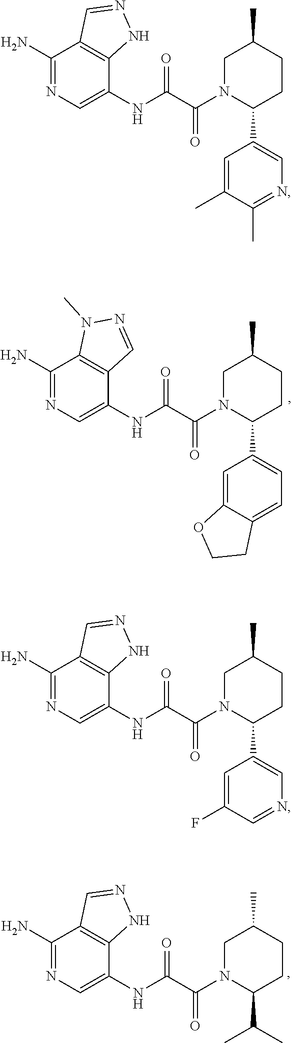 [SMILES: Cc1cc([C@H]2CC[C@H](C)CN2C(=O)C(=O)Nc2cnc(N)c3cn[nH]c23)cnc1C ; C[C@H]1CC[C@H](c2ccc3c(c2)OCC3)N(C(=O)C(=O)Nc2cnc(N)c3c2cnn3C)C1 ; C[C@H]1CC[C@H](c2cncc(F)c2)N(C(=O)C(=O)Nc2cnc(N)c3cn[nH]c23)C1 ; CC(C)[C@@H]1CC[C@@H](C)CN1C(=O)C(=O)Nc1cnc(N)c2cn[nH]c12] 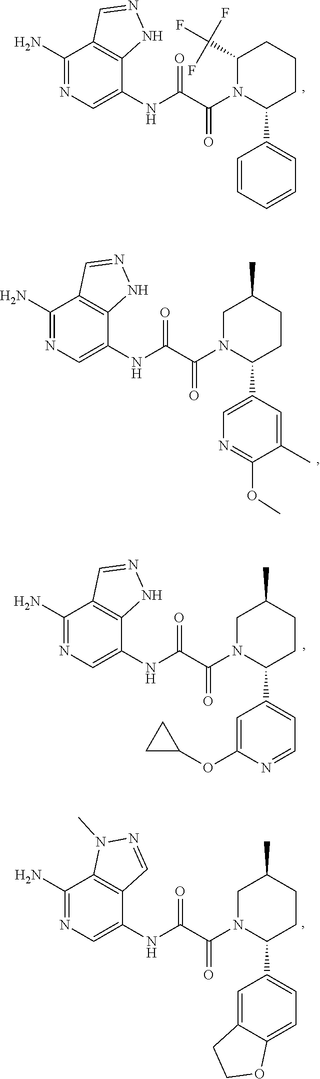 [SMILES: Nc1ncc(NC(=O)C(=O)N2[C@@H](c3ccccc3)CCC[C@H]2C(F)(F)F)c2[nH]ncc12 ; COc1ncc([C@H]2CC[C@H](C)CN2C(=O)C(=O)Nc2cnc(N)c3cn[nH]c23)cc1C ; C[C@H]1CC[C@H](c2ccnc(OC3CC3)c2)N(C(=O)C(=O)Nc2cnc(N)c3cn[nH]c23)C1 ; C[C@H]1CC[C@H](c2ccc3c(c2)CCO3)N(C(=O)C(=O)Nc2cnc(N)c3c2cnn3C)C1] 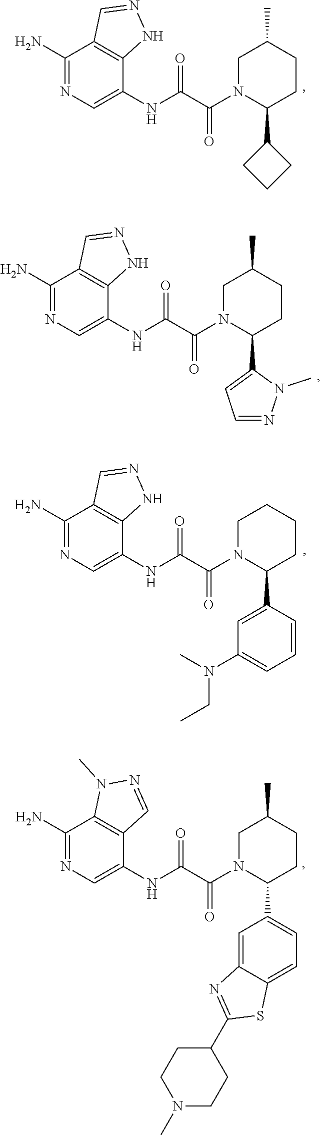 [SMILES: C[C@@H]1CC[C@@H](C2CCC2)N(C(=O)C(=O)Nc2cnc(N)c3cn[nH]c23)C1 ; C[C@H]1CC[C@@H](c2ccnn2C)N(C(=O)C(=O)Nc2cnc(N)c3cn[nH]c23)C1 ; CCN(C)c1cccc([C@@H]2CCCCN2C(=O)C(=O)Nc2cnc(N)c3cn[nH]c23)c1 ; C[C@H]1CC[C@H](c2ccc3sc(C4CCN(C)CC4)nc3c2)N(C(=O)C(=O)Nc2cnc(N)c3c2cnn3C)C1] 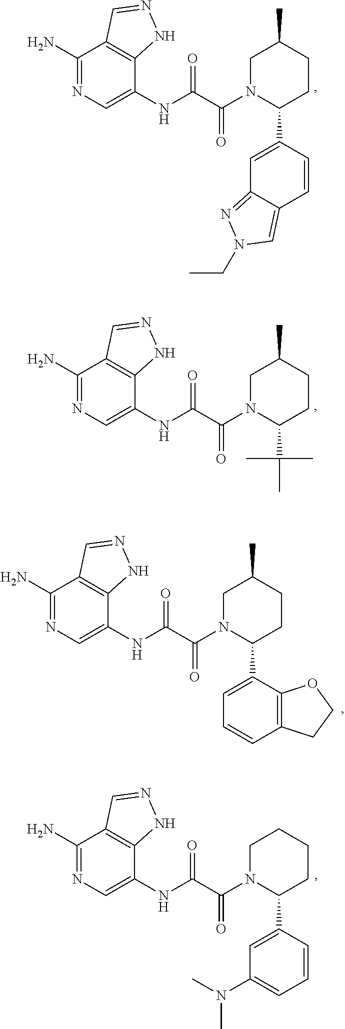 [SMILES: CCn1cc2ccc([C@H]3CC[C@H](C)CN3C(=O)C(=O)Nc3cnc(N)c4cn[nH]c34)cc2n1 ; C[C@H]1CC[C@H](C(C)(C)C)N(C(=O)C(=O)Nc2cnc(N)c3cn[nH]c23)C1 ; C[C@H]1CC[C@H](c2cccc3c2OCC3)N(C(=O)C(=O)Nc2cnc(N)c3cn[nH]c23)C1 ; CN(C)c1cccc([C@H]2CCCCN2C(=O)C(=O)Nc2cnc(N)c3cn[nH]c23)c1] 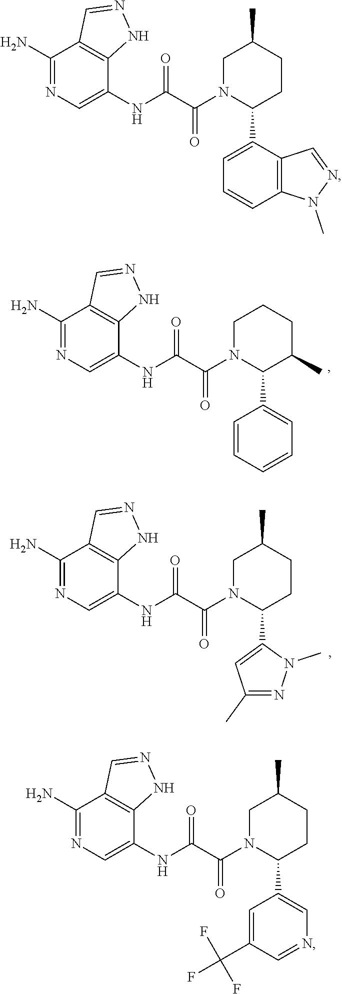 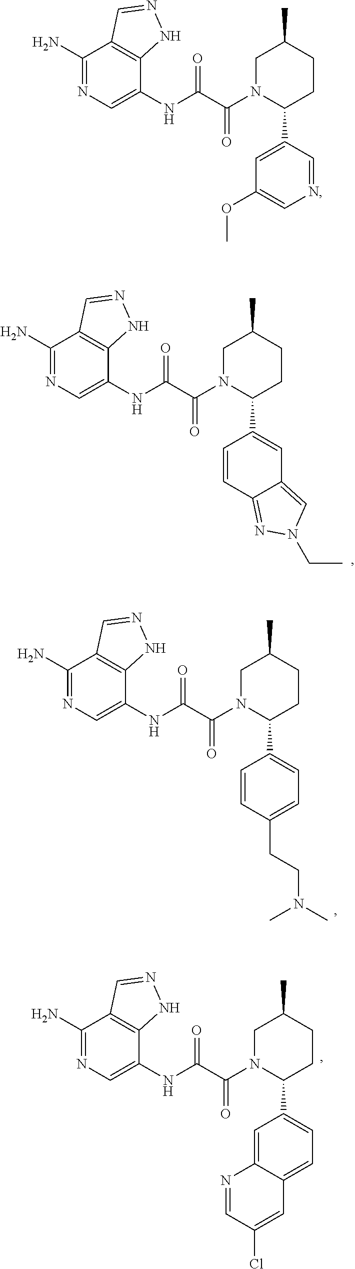 [SMILES: COc1cncc([C@H]2CC[C@H](C)CN2C(=O)C(=O)Nc2cnc(N)c3cn[nH]c23)c1 ; CCn1cc2cc([C@H]3CC[C@H](C)CN3C(=O)C(=O)Nc3cnc(N)c4cn[nH]c34)ccc2n1 ; C[C@H]1CC[C@H](c2ccc(CCN(C)C)cc2)N(C(=O)C(=O)Nc2cnc(N)c3cn[nH]c23)C1 ; C[C@H]1CC[C@H](c2ccc3cc(Cl)cnc3c2)N(C(=O)C(=O)Nc2cnc(N)c3cn[nH]c23)C1] 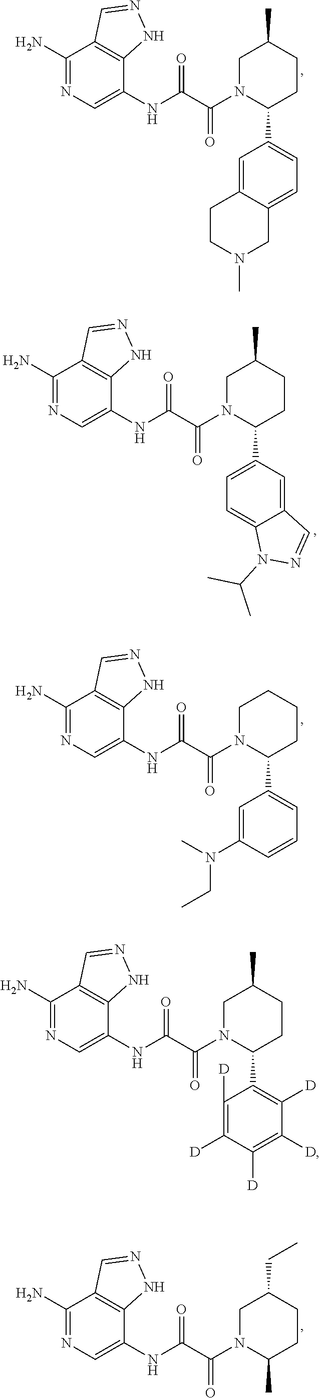 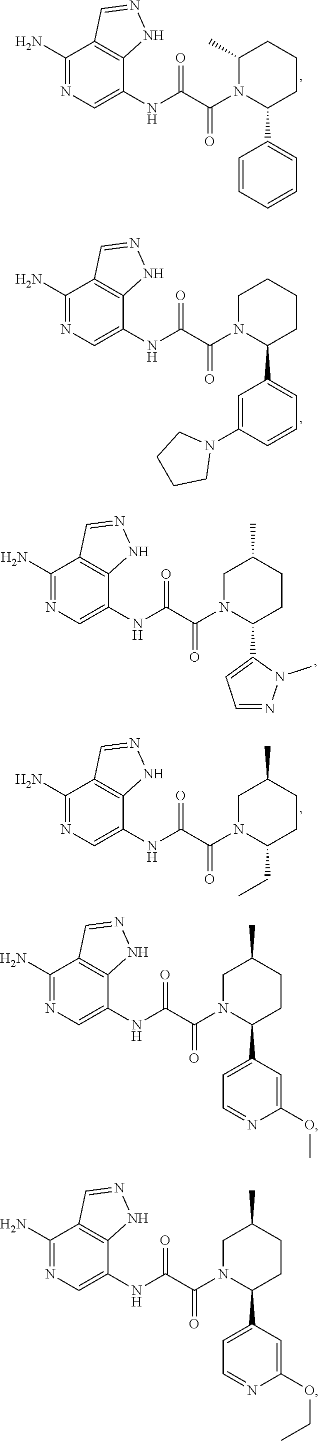 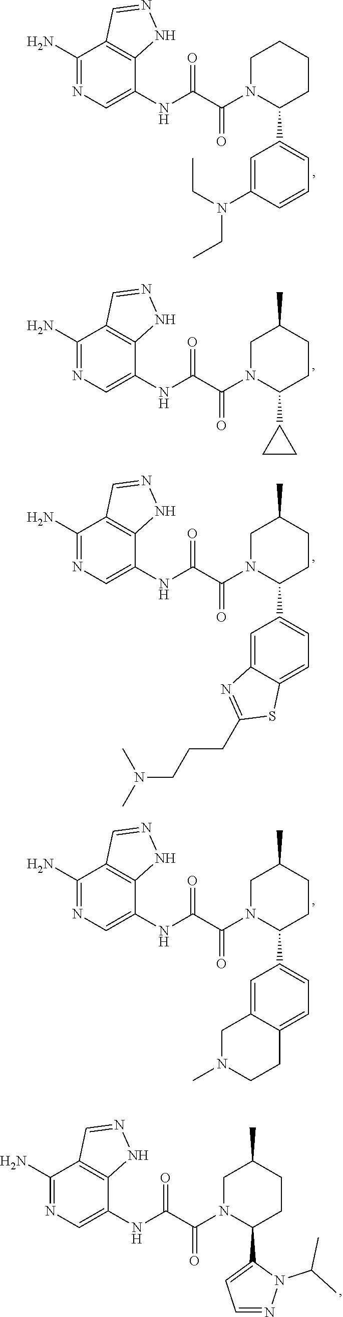 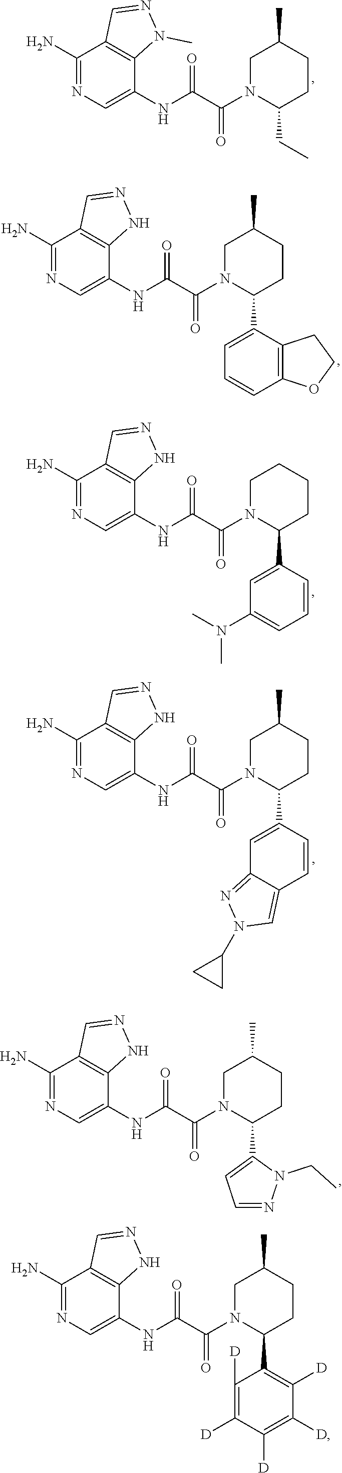 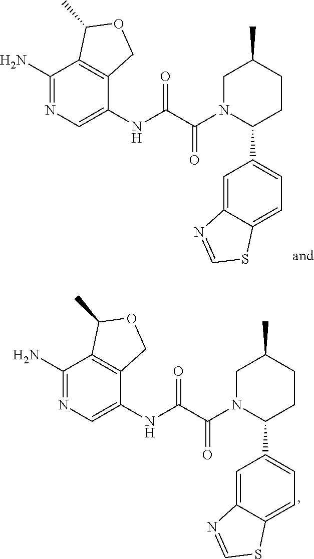 [SMILES: C[C@H]1CC[C@H](c2ccc3scnc3c2)N(C(=O)C(=O)Nc2cnc(N)c3c2CO[C@H]3C)C1 ; C[C@H]1CC[C@H](c2ccc3scnc3c2)N(C(=O)C(=O)Nc2cnc(N)c3c2CO[C@@H]3C)C1]
or a pharmaceutically acceptable salt thereof.

70. A pharmaceutical composition comprising a compound of any one of claims 1 to 69, or a pharmaceutically acceptable salt thereof, and a pharmaceutically acceptable carrier.

71. The pharmaceutical composition of claim 70, further comprising a second therapeutic agent.

72. A compound of any one of claims 1 to 69, or a pharmaceutically acceptable salt thereof, or a pharmaceutically acceptable composition of claim 70 for treating an MTAP-deficient and/or an MTA-accumulating disease in a subject in need thereof.

73. The compound or composition for use of claim 72 wherein the compound, or a pharmaceutically acceptable salt thereof, or composition is configured to be administered in combination with a second therapeutic agent.

74. A pharmaceutically acceptable composition of claim 71 for use in treating an MTAP-deficient and/or an MTA-accumulating disease in a subject in need thereof.

75. The compound or composition for use of any one of claims 72 to 74 wherein the disease is a proliferating disease.

76. The compound or composition for use of claim 75 wherein the disease is an MTAP-deficient and/or MTA-accumulating cancer.

77. The compound or composition for use of claim 76 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

78. Use of a compound of any one of claims 1 to 69, or a pharmaceutically acceptable salt thereof, or of a pharmaceutically acceptable composition of claim 70 in the manufacturing of a medicament for treating an MTAP-deficient and/or an MTA-accumulating disease in a subject in need thereof.

79. The use of claim 78 wherein the medicament is configured to be administered in combination with a second therapeutic agent.

80. Use of a pharmaceutically acceptable composition of claim 71 in the manufacturing of a medicament for treating an MTAP-deficient and/or an MTA-accumulating disease in a subject in need thereof.

81. The use of any one of claims 78 to 80 wherein the disease is a proliferating disease.

82. The use of claim 81 wherein the disease is an MTAP-deficient and/or MTA-accumulating cancer.

83. The use of claim 82 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.
